(19) United States
(12) Patent Application Publication
(10) Pub. No.: US 2020/0109395 A1
(43) Pub. Date: Apr. 9, 2020

(54) GENETIC SYSTEMS THAT DEFEND AGAINST FOREIGN DNA AND USES THEREOF

(71) Applicant: YEDA RESEARCH AND DEVELOPMENT CO. LTD., Rehovot (IL)

(72) Inventors: Rotem SOREK, Rehovot (IL); Gal OFiR, Rehovot (IL); Gil AMITAI, Rehovot (IL); Sarah MELAMED, Rehovot (IL); Shany DORON, Rehovot (IL); Azita LEAVITT, Rehovot (IL)

(73) Assignee: YEDA RESEARCH AND DEVELOPMENT CO. LTD., Rehovot (IL)

(21) Appl. No.: 16/614,370

(22) PCT Filed: May 23, 2018
(86) PCT No.: PCT/IL2018/050559

§ 371 Date: Nov. 18, 2019

Related U.S. Application Data
(60) Provisional application No. 62/586,911, filed on Nov. 16, 2017.
(60) Provisional application No. 62/512,219, filed on May 30, 2017.
(60) Provisional application No. 62/512,216, filed on May 30, 2017.

(51) Int. Cl. C12N 15/10; C07K 14/195; C12N 15/74; G16B 20/00; G16B 40/00

(52) U.S. Cl. CPC C12N 15/102; C07K 14/195; C07K 2319/00; G16B 20/00; G16B 40/00; C12N 15/74

(57) ABSTRACT

Disclosed herein are microbial defense systems, which provide cells protection against phage infection and plasmid transformation. Methods of use of these defense systems for protecting cells from phage infection and plasmid transformation are also disclosed, wherein defense systems may be used individually or in combination. In addition, disclosed herein are methods of making cells that express these defense systems.

### SEQUENCE LISTING STATEMENT

The instant application contains a “lengthy” Sequence Listing which has been submitted via CD-R in lieu of a printed paper copy, and is hereby incorporated by reference in its entirety. Said CD-R, recorded on May 15, 2018, are labeled “CRF”, “COPY 1—SEQUENCE LISTING PART,” “COPY 2—SEQUENCE LISTING PART” and “COPY 3—SEQUENCE LISTING PART,” respectively, and each contains only one identical 350,356,650 bytes file (P-573737-PC_SL.txt).

### FIELD OF DISCLOSURE

Microbial defense systems providing host cells with anti-phage resistance or resistance against plasmid transformation, or both are disclosed. Methods of identifying these systems, and using these systems for example but not limited, for producing bacterial cells comprising defense systems, for producing food products, and for gene editing, are also disclosed.

### BACKGROUND

Bacteria and archaea are often being attacked by viruses (phages), and as a result have developed multiple, sophisticated lines of active defense that can collectively be referred to as the prokaryotic “immune system”. Anti-phage defense strategies include restriction-modification (R/M) systems that target specific sequences on the invading phage, CRISPR-Cas that provides acquired immunity through memorization of past phage attacks, infection systems (Abi) that lead the cell to death or metabolic arrest upon infection, and additional systems whose mechanism of action is not yet clear such as BREX and DISARM. Different bacteria encode different sets of defense systems: CRISPR-Cas systems are found in about 40% of all sequenced bacteria, BREX appears in 10% of sequenced bacteria, and DISARM in about 1%. It has been suggested that many new, currently unknown defense systems reside in the genomes of non-model bacteria and archaea and await discovery.

Anti-phage defense systems have been found to be frequently physically clustered in bacterial and archaeal genomes such that, for example, genes encoding restriction enzymes commonly reside in the vicinity of genes encoding infection systems and other phage resistance systems. The observation that defense systems are clustered in genomic “defense islands” has led to the suggestion that genes of unknown function residing within such defense islands may also participate in anti-phage defense. Indeed, recent studies that anecdotally examined individual genes enriched next to known defense genes yielded the discovery of new systems that protect bacteria against phages.

Anti-phage defense systems are usually composed of multiple genes that work in concert to achieve defense—for example, cas1, cas2, cas3 and the cascade genes in type I CRISPR-Cas systems, and the R, M and S genes in type I restriction systems. Genes functioning within the same defense system are very frequently encoded on the same operon, and the gene order within the operon is highly conserved among distantly related organisms sharing the same system.

Some of these phage-resistance systems have been successfully adapted for biotechnological application; for example, the R/M and the CRISPR/Cas systems have been extensively used for genetic engineering purposes in general and gene therapy in particular.

A broad array of food products, commodity chemicals, and biotechnology products are manufactured industrially by large-scale bacterial fermentation of various substrates. Enormous amounts of bacteria are being cultivated each day in large fermentation vats, thus foreign nucleic acid contamination, for example phage contamination can rapidly bring fermentations to a halt and cause economic setbacks, and is therefore considered a serious threat in these industries. The dairy fermentation industry has openly acknowledged the problem of phages and has been working with academia and starter culture companies to develop defense strategies and systems to curtail the propagation and evolution of phages for decades. There remains a need to provide bacteria that can defend against nucleic acid molecules from foreign sources.

The use of defense systems, for example anti-phage may provide the necessary defense for bacteria in food, chemical, and biotechnology production. Thus, it would be advantageous to both the producers and the consumers to have bacteria resistant to nucleic acid molecules from foreign sources, for example with anti-phage, anti-conjugative element, and or anti-plasmid activity.

Disclosed herein are newly identified anti-phage defense systems, anti-conjugative element systems, and an anti-plasmid system that show strong protection against nucleic acid molecules from foreign sources. These systems could provide protection for fermentation bacteria against phages, for protection of bacteria against viruses, and may also provide biotechnological tools for use with prokaryotic and eukaryotic cells.

### SUMMARY

In one aspect, disclosed herein is a method of protecting bacteria from foreign nucleic acid invasion comprising a step of introducing into the bacteria at least one defense system (a) to (o), said at least one defense system comprising:
(a) a Defense System IV comprising at least two different polypeptide components selected from
   a HamA polypeptide comprising a pfam08878 domain or a DUF1837 domain or a combination thereof, or comprising an amino acid sequence having at least 80% identity to a sequence selected from the group referenced in Table 12 rows 2-1782 columns H and I; and
   a HamB polypeptide comprising COG1204 domain or a pfam00270 domain or a pfam00271 domain or any combination thereof, or comprising an amino acid sequence having at least 80% identity to a sequence selected from the group referenced in Table 12 rows 2-1782 columns L and M; or
(b) a Defense System Ia comprising at least two different polypeptide components selected from
   a ZorA polypeptide comprising a pfam01618 domain, or comprising an amino acid sequence having at least 80% identity to a sequence selected from the group referenced in Table 8 rows 2-1830 columns J and K;
   a ZorB polypeptide comprising a pfam13677 domain or a pfam00691 domain or a combination thereof, or comprising an amino acid sequence having at least 80% identity to a sequence selected from the group referenced in Table 8 rows 2-1830 columns N and O;
   a ZorC polypeptide comprising a pfam15611 domain, or comprising an amino acid sequence having at least 80% identity to a sequence selected from the group referenced in Table 8 rows 2-1174 columns R and S; and
   a ZorD polypeptide comprising a pfam00176 domain or a pfam00271 domain or a combination thereof, or comprising an amino acid sequence having at least 80% identity to a sequence selected from the group referenced in Table 8 rows 2-1174 columns V and W;
   or
(c) a Defense System Ib comprising a ZorE polypeptide comprising a pfam01844 domain or a COG3183 domain or a combination thereof, or comprising an amino acid sequence having at least 80% identity to a sequence selected from the group referenced in Table 8 rows 1175-1830 columns Z and AA; and at least one other polypeptide component selected from
   a ZorA polypeptide comprising a pfam01618 domain, or comprising an amino acid sequence having at least 80% identity to a sequence selected from the group referenced in Table 8 rows 2-1830 columns J and K; and
   a ZorB polypeptide comprising a pfam13677 domain or a pfam00691 domain or a combination thereof, or comprising an amino acid sequence having at least 80% identity to a sequence selected from the group referenced in Table 8 rows 2-1830 columns N and O; or
(d) a Defense System II comprising at least two different polypeptide components selected from
   a ThsA polypeptide comprising a pfam13289 domain or a pfam14519 domain or a combination thereof, or comprising an amino acid sequence having at least 80% identity to a sequence selected from the group referenced in Table 9 rows 2-2100 columns I and J; and
   a ThsB polypeptide comprising a pfam13676 domain or a pfam08937 domain or a pfam08357 domain or any combination thereof, or comprising an amino acid sequence having at least 80% identity to a sequence selected from the group referenced in Table 9 rows 2-2100 columns M and N, Q and R, U and V, Y and Z, and AC and AD; or
(e) a Defense System IIIa comprising at least two different polypeptide components selected from
   a DruA polypeptide comprising a DUF4338 domain or a pfam14236 domain or a combination thereof; or comprising an amino acid sequence having at least 80% identity to a sequence selected from the group referenced in Table 10 rows 2-123 columns J and K, or a combination thereof;
   a DruB polypeptide comprising an amino acid sequence having at least 80% identity to a sequence selected from the group referenced in Table 10 rows 2-123 columns N and O;
   a DruC polypeptide comprising an amino acid sequence having at least 80% identity to a sequence selected from the group referenced in Table 10 rows 2-123 columns R and S;
   a DruD polypeptide comprising an amino acid sequence having at least 80% identity to a sequence selected from the group referenced in Table 10 rows 2-123 columns V and W; and
   a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA; or
(f) a Defense System IIIb comprising at least two different polypeptide components selected from
   a DruM polypeptide comprising a pfam00145 domain or a COG0270 domain or a combination thereof, or comprising an amino acid sequence having at least 80% identity to a sequence selected from the group referenced in Table 10 rows 124-295 columns AD and AE;
   a DruF polypeptide comprising an amino acid sequence having at least 80% identity to a sequence selected from the group referenced in Table 10 rows 124-295 columns AH and AI;
   a DruG polypeptide comprising an amino acid sequence having at least 80% identity to a sequence selected from the group referenced in Table 10 rows 124-295 columns AL and AM; and
   a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 column Z and AA; or
(g) a Defense System IIIc comprising at least two different polypeptide components selected from
   a DruH polypeptide comprising an amino acid sequence having at least 80% identity to a sequence selected from the group referenced in Table 10 rows 296-1343 columns AP and AQ; and
   a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA; or
(h) a Defense System V comprising a SduA polypeptide comprising a pfam14082 domain or a pfam01939 domain or a combination thereof, or comprising an amino acid sequence having at least 80% identity to a sequence selected from the group referenced in Table 13 rows 2-1247 columns H and I; or
(i) a Defense System VI comprising at least two different polypeptide components selected from
   a GajA polypeptide comprising a pfam13175 domain or a COG3593 domain or a combination thereof, or comprising an amino acid sequence having at least 80% identity to a sequence selected from the group referenced in Table 14 rows 2-4599 columns H and I; and
   a GajB polypeptide comprising a pfam04257 domain or a pfam00580 domain or a pfam13361 domain or any combination thereof, or comprising an amino acid sequence having at least 80% identity to a sequence selected from the group referenced in Table 14 rows 2-4599 columns L and M; or
(j) a Defense System VII comprising at least two different polypeptide components selected from
   a PtuA polypeptide comprising a pfam13304 domain or a pfam02463 domain or a combination thereof, or comprising an amino acid sequence having at least 80% identity to a sequence selected from the group referenced in Table 15 rows 2-12507 columns H and I; and
   a PtuB polypeptide comprising pfam13395 domain or a pfam 01844 domain or a combination thereof, or comprising an amino acid sequence having at least 80% identity to a sequence selected from the group referenced in Table 15 rows 2-2507 columns L and M; or
(k) a Defense System VIII comprising at least two different polypeptide components selected from
   a LmuA polypeptide comprising a pfam14130 domain or a DUF4297 domain or a combination thereof, or comprising an amino acid sequence having at least 80% identity to a sequence selected from the group referenced in Table 16 rows 2-698 columns H and I; and
   a LmuB polypeptide comprising a pfam02463 domain, or comprising an amino acid sequence having at least 80% identity to a sequence selected from the group referenced in Table 16 rows 2-698 columns L and M; or
(l) a Defense System IX comprising at least two different polypeptide components selected from
   a KwaA polypeptide comprising an amino acid sequence having at least 80% identity to a sequence selected from the group referenced in Table 17 rows 2-935 columns H and I; and
   a KwaB polypeptide comprising a pfam16162 domain or a DUF4868 domain or a combination thereof; or comprising an amino acid sequence having at least 80% identity to a sequence selected from the group referenced in Table 17 rows 2-935 columns L and M; or
(m) a Defense System Xa comprising at least two different polypeptide components selected from
   a JetA polypeptide comprising a pfam11855 domain or a DUF3375 domain or a combination thereof, or comprising an amino acid sequence having at least 80% identity to a sequence selected from the group referenced in Table 11 rows 2-2322 columns J and K;
   a JetB polypeptide comprising a pfam13835 domain or a DUF4194 domain or a combination thereof, or comprising an amino acid sequence having at least 80% identity to a sequence selected from the group referenced in Table 11 rows 2-2322 columns N and O;
   a JetC polypeptide comprising a pfam13555 domain or a pfam13558 domain or a COG4913 domain or any combination thereof; or comprising an amino acid sequence having at least 80% identity to a sequence selected from the group referenced in Table 11 rows 2-2322 columns R and S; and
   a JetD polypeptide comprising a pfam11795 domain or a DUF3322 domain or a pfam09983 domain or a DUF2220 domain or a COG4924 domain or any combination thereof, or comprising an amino acid sequence having at least 80% identity to a sequence selected from the group referenced in Table 11 rows 2-2322 columns V and W; or
(n) a Defense System Xb comprising at least two different polypeptide components selected from
   a JetAⁱⁱ polypeptide comprising a pfam09660 domain or a DUF2397 domain or a combination thereof, or comprising an amino acid sequence having at least 80% identity to a sequence selected from the group referenced in Table 11 rows 2323-2844 columns J and K;
   a JetBⁱⁱ polypeptide comprising a pfam09661 domain or a DUF2398 domain or a combination thereof, or comprising an amino acid sequence having at least 80% identity to a sequence selected from the group referenced in Table 11 rows 2323-2844 columns N and O;
   a JetCⁱⁱ polypeptide comprising a pfam13558 domain, or comprising an amino acid sequence having at least 80% identity to a sequence selected from the group referenced in Table 11 rows 2323-2844 columns R and S; and
   a JetDⁱⁱ polypeptide comprising a pfam11796 domain or a DUF3323 domain or a pfam09664 domain or a DUF2399 domain or a combination thereof, or comprising an amino acid sequence having at least 80% identity to a sequence selected from the group referenced in Table 11 rows 2323-2844 columns V and W; or
(o) a Defense System Xc comprising at least two different polypeptide components selected from
   a JetAⁱⁱⁱ polypeptide comprising an amino acid sequence having at least 80% identity to a sequence selected from the group referenced in Table 11 rows 2845-3174 columns and K;
   a JetBⁱⁱⁱ polypeptide comprising an amino acid sequence having at least 80% identity to a sequence selected from the group referenced in Table 11 rows 2845-3174 columns N and O;
   a JetCⁱⁱⁱ polypeptide comprising a COG1196 domain or comprising an amino acid sequence having at least 80% identity to a sequence selected from the group referenced in Table 11 rows 2845-3174 columns R and S; and
   a JetDⁱⁱⁱ polypeptide comprising a pfam09983 domain or a pfam09664 domain or a DUF2220 or a DUF2399 domain or any combination thereof, or comprising an amino acid sequence having at least 80% identity to a sequence selected from the group referenced in Table 11 rows 2845-3174 columns V and W; or
(p) any combination of Defense Systems Ia-Xc (a)-(o);
wherein the introduction of said at least one defense system results in the bacteria being protected from foreign DNA invasion.

In a related aspect, methods of protecting bacteria from foreign nucleic acid invasion comprises protecting from phage infection, protection from plasmid transformation, or protecting from entry of conjugative elements, or any combination thereof. In another related aspect, a phage comprises a single stranded DNA (ssDNA) phage, a double stranded DNA (dsDNA) phage, a single stranded RNA (ssRNA) phage, a double stranded RNA (dsRNA) phage, a lytic phage, or a lysogenic phage, or a combination thereof. In another related aspect, at least one defense system comprises
(a) a Defense System IV comprising the HamA polypeptide and the HamB polypeptide; or
(b) a Defense System Ia comprising the ZorA polypeptide, the ZorB polypeptide, the ZorC polypeptide, and the ZorD polypeptide; or
(c) a Defense System Ib comprising the ZorA polypeptide, the ZorB polypeptide, and the ZorE polypeptide; or
(d) a Defense System II comprising the ThsA polypeptide, and the ThsB polypeptide; or
(e) a Defense System IIIa comprising the DruA polypeptide, the DruB polypeptide, the DruC polypeptide, the DruD polypeptide, and the DruE polypeptide; or
(f) a Defense System IIIb comprising the DruM polypeptide, the DruF polypeptide, the DruG polypeptide, and the DruE polypeptide; or
(g) a Defense System IIIc comprising the DruH polypeptide and the DruE polypeptide; or
(h) a Defense System V comprising the SduA polypeptide; or
(i) a Defense System VI comprising the GajA polypeptide and the GajB polypeptide; or
(j) a Defense System VII comprising the PtuA polypeptide and the PtuB polypeptide; or
(k) a Defense System VIII comprising the LmuA polypeptide and the LmuB polypeptide; or
(l) a Defense System IX comprising the KwaA polypeptide and the KwaB polypeptide; or
(m) a Defense System Xa comprising the JetA polypeptide, the JetB polypeptide, the JetC polypeptide, and the JetD polypeptide; or
(n) a Defense System Xb comprising the JetAⁱⁱ polypeptide, the JetBⁱⁱ polypeptide, the JetCⁱⁱ polypeptide, and the JetDⁱⁱ polypeptide; or
(o) a Defense System Xc comprising the JetAⁱⁱⁱ polypeptide, the JetBⁱⁱⁱ polypeptide, the JetCⁱⁱⁱ polypeptide, and the JetDⁱⁱⁱ polypeptide; or
(p) any combination of Defense Systems Ia-Xc (a)-(o).

In another related aspect, defense systems disclosed herein may be encoded by a nucleic acid sequence, wherein
(a) said Defense System IV is encoded by
   a nucleic acid sequence selected from the group referenced in Table 12, rows 2-1782, columns G, P, and Q or the nucleic acid sequence set forth in SEQ ID NO: 4; or
   a nucleic acid sequence encoding the at least two different polypeptide sequences as disclosed herein; or
   a nucleic acid sequence encoding the HamA polypeptide and HamB polypeptide as disclosed herein;
(b) said Defense System Ia is encoded by
   a nucleic acid sequence selected from the group referenced in Table 8 rows 2-1174 columns G, AD, and AE or the nucleic acid sequence set forth in SEQ ID NO: 14; or
   a nucleic acid sequence encoding the at least two different polypeptide sequences as disclosed herein; or
   a nucleic acid sequence encoding the ZorA polypeptide, the ZorB polypeptide, the ZorC polypeptide, and ZorD polypeptide as disclosed herein;
(c) said Defense System Ib is encoded by
   a nucleic acid sequence selected from the group referenced in Table 8 rows 1175-1830 columns G, AD, and AE or set forth in SEQ ID NO: 15; or a nucleic acid sequence encoding the at least two different polypeptide sequences as disclosed herein; or
   a nucleic acid sequence encoding the ZorA polypeptide, the ZorB polypeptide, and ZorE polypeptide as disclosed herein;
(d) said Defense System II is encoded by
   a nucleic acid sequence selected from the group referenced in Table 9 rows 2-2100 columns G, AG, and AH or set forth in the nucleic acid sequence set forth in SEQ ID NO: 10 or SEQ ID NO: 11; or
   a nucleic acid sequence encoding the at least two different polypeptide sequences as disclosed herein; or
   a nucleic acid sequence encoding the ThsA polypeptide and the ThsB polypeptide as disclosed herein;
(e) said Defense System IIIa is encoded by
   a nucleic acid sequence selected from the group referenced in Table 10 rows 2-123 columns G, AT, and AU or set forth in SEQ ID NO: 16; or
   a nucleic acid sequence encoding the at least two different polypeptide sequences as disclosed herein; or
   a nucleic acid sequence encoding the DruA polypeptide, the DruB polypeptide, the DruC, the DruD, and the DruE polypeptide as disclosed herein;
(f) said Defense System IIIb is encoded by
   a nucleic acid sequence selected from the group referenced in Table 10 rows 124-295 columns G, AT, AU; or
   a nucleic acid sequence encoding the at least two different polypeptide sequences as disclosed herein; or
   a nucleic acid sequence encoding the DruM polypeptide, the DruF polypeptide, the DruG polypeptide, and the DruE polypeptide as disclosed herein;
(g) said Defense System IIIc is encoded by
   a nucleic acid sequence selected from the group referenced in Table 10 rows 296-1343 columns G, AT, and AU; or
   a nucleic acid sequence encoding the at least two different polypeptide sequences as disclosed herein; or
   a nucleic acid sequence encoding the DruH polypeptide and the DruE polypeptide as disclosed herein;
(h) said Defense System V is encoded by
   a nucleic acid sequence selected from the group referenced in Table 13, rows 2-1247, columns H and J; or
   a nucleic acid sequence encoding the SduA polypeptide as disclosed herein;
(i) said Defense System VI is encoded by
   a nucleic acid sequence selected from the group referenced in Table 14, rows 2-4599, columns G, P, and Q or the sequences set forth in SEQ ID NO: 5 and SEQ ID NO: 6; or
   a nucleic acid sequence encoding the at least two different polypeptide sequences as disclosed herein; or
   a nucleic acid sequence encoding the GajA polypeptide and the GajB polypeptide as disclosed herein;
(j) said Defense System VII is encoded by
   a nucleic acid sequence selected from the group referenced in Table 15, rows 2-2507, columns G, P, and Q or the sequences set forth in SEQ ID NO: 12 and SEQ ID NO: 13; or
   a nucleic acid sequence encoding the at least two different polypeptide sequences as disclosed herein; or
   a nucleic acid sequence encoding the Ptu polypeptide and the PtuB polypeptide as disclosed herein;
(k) said Defense System VIII is encoded by
   a nucleic acid sequence selected from the group referenced in Table 16, rows 2-698, columns G, P, and Q or the sequences set forth in SEQ ID NO: 7 and SEQ ID NO: 8; or
   a nucleic acid sequence encoding the at least two different polypeptide sequences as disclosed herein; or
   a nucleic acid sequence encoding the LmuA polypeptide and LmuB polypeptide as disclosed herein;
(l) said Defense System IX is encoded by
   a nucleic acid sequence selected from the group referenced in Table 17, rows 2-935, columns G, P, and Q or the sequence set forth in SEQ ID NO: 3; or
   a nucleic acid sequence encoding the at least two different polypeptide sequences as disclosed herein; or
   a nucleic acid sequence encoding the KwaA polypeptide and the KwaB polypeptide as disclosed herein;
(m) said Defense System Xa is encoded by
   a nucleic acid sequence selected from the group referenced in Table 11, rows 2-2322, columns G, Z, and AA or the sequence set forth in SEQ ID NO: 17; or
   a nucleic acid sequence encoding the at least two different polypeptide sequences as disclosed herein; or
   a nucleic acid sequence encoding the JetA polypeptide, the JetB polypeptide, the JetC polypeptide, and the JetD polypeptide as disclosed herein;
(n) said Defense System Xb is encoded by
   a nucleic acid sequence having at least 80% homology to a sequence selected from the group referenced in Table 11, rows 2323-2844, columns G, Z, and AA or the sequence set forth in SEQ ID NO: 18; or
   a nucleic acid sequence encoding the at least two different polypeptide sequences as disclosed herein; or
   a nucleic acid sequence encoding the JetAⁱⁱ polypeptide, the JetBⁱⁱ polypeptide, the JetCⁱⁱ polypeptide, and the JetDⁱⁱ polypeptide as disclosed herein;
(o) said Defense System Xc is encoded by
   a nucleic acid sequence selected from the group referenced in Table 11, rows 2845-3174, columns G, Z, and AA or the sequence set forth in SEQ ID NO: 19; or
   a nucleic acid sequence encoding the at least two different polypeptide sequences as disclosed herein; or
   a nucleic acid sequence encoding the JetAⁱⁱⁱ polypeptide, the JetBⁱⁱⁱ polypeptide, the JetCⁱⁱⁱ polypeptide, and the JetDⁱⁱⁱ polypeptide as disclosed herein.

In another related aspect, methods disclosed herein comprise use of a defense system as disclosed or use of a combination of defense systems wherein
(a) for said Defense system IV, said HamA polypeptide is encoded by a nucleic acid sequence selected from the group referenced in Table 12, rows 2-1782, columns H and J, or
   said HamB polypeptide is encoded by a nucleic acid sequence selected from the group referenced in Table 12, rows 2-1782, columns L and N, or a combination thereof;
(b) for said Defense system Ia, said ZorA polypeptide is encoded by a nucleic acid sequence selected from the group referenced in Table 8 rows 2-1830 columns J and L,
   said ZorB polypeptide is encoded by a nucleic acid sequence selected from the group referenced in Table 8 rows 2-1830 columns N and P,
   said ZorC polypeptide is encoded by a nucleic acid sequence selected from the group referenced in Table 8 rows 2-1174 columns R and T, or
   said ZorD polypeptide is encoded by a nucleic acid sequence selected from the group referenced in Table 8 rows 2-1174 columns V and X, or a combination thereof;
(c) for said Defense system Ib, said ZorA polypeptide is encoded by a nucleic acid sequence selected from the group referenced in Table 8 rows 2-1830 columns J and L,
   said ZorB polypeptide is encoded by a nucleic acid sequence selected from the group Table 8 rows 2-1830 columns N and P,
   said ZorE polypeptide is encoded by a nucleic acid sequence selected from the group referenced in Table 8 rows 1175-1830 columns Z and AB, or
   a combination thereof;
(d) for said Defense system II, said ThsA polypeptide is encoded by a nucleic acid sequence selected from the group referenced in Table 9 rows 2-2100 columns I and K,
   said ThsB polypeptide is encoded by a nucleic acid sequence selected from the group referenced in Table 9 rows 2-2100 columns M and O, Q and S, U and W, Y and AA, and AC and AE, or
   a combination thereof;
(e) for said Defense system IIIa, said DruA polypeptide is encoded by a nucleic acid sequence selected from the group referenced in Table 10 rows 2-123 columns J and L, or
   said DruB polypeptide is encoded by a nucleic acid sequence selected from the group referenced in Table 10 rows 2-123 columns N and P, or
   said DruC polypeptide is encoded by a nucleic acid sequence selected from the group referenced in Table 10 rows 2-123 columns R and T, or
   said DruD polypeptide is encoded by a nucleic acid sequence selected from the group referenced in Table 10 rows 2-123 columns V and X, or
   said DruE polypeptide is encoded by a nucleic acid sequence selected from the group referenced in Table 10 rows 2-1343 columns Z and AB, or
   a combination thereof;
(f) for said Defense system IIIb, said DruM polypeptide is encoded by a nucleic acid sequence selected from the group referenced in Table 10 rows 124-295 columns AD and AF, or
   said DruF polypeptide is encoded by a nucleic acid sequence selected from the group referenced in Table 10 rows 124-295 columns AH and AJ, or
   said DruG polypeptide is encoded by a nucleic acid sequence selected from the group referenced in Table 10 rows 124-295 columns AL and AN, or
   said DruE polypeptide is encoded by a nucleic acid sequence selected from the group referenced in Table 10 rows 2-1343 columns Z and AB, or
   a combination thereof;
(g) for said Defense system IIIc, said DruH polypeptide is encoded by a nucleic acid sequence selected from the group referenced in Table 10 rows 296-1343 columns AP and AR, or
   said DruE polypeptide is encoded by a nucleic acid sequence selected from the group referenced in Table 10 rows 2-1343 columns Z and ZB, or a combination thereof;
(h) for said Defense system V, said SduA polypeptide is encoded by a nucleic acid sequence selected from the group referenced in Table 13; rows 2-1247, columns H and J;
(i) for said Defense system VI, said GajA polypeptide is encoded by a nucleic acid sequence selected from the group referenced in Table 14, rows 2-4599, columns H and J, or
   said GajB polypeptide is encoded by a nucleic acid sequence selected from the group referenced in Table 14, rows 2-4599, columns L and N, or a combination thereof;
(j) for said Defense system VII, said PtuA polypeptide is encoded by a nucleic acid sequence selected from the group referenced in Table 15, rows 2-2507, columns H and J, or
   said PtuB polypeptide is encoded by a nucleic acid sequence selected from the group referenced in Table 15, rows 2-2507, columns L and N, or a combination thereof;
(k) for said Defense system VIII, said LmuA polypeptide is encoded by a nucleic acid sequence selected from the group referenced in Table 16, rows 2-698, columns H and J, or
   said LmuB polypeptide is encoded by a nucleic acid sequence selected from the group referenced in Table 16, rows 2-698, columns L and N, or a combination thereof;
(l) for said Defense system IX, said KwaA polypeptide is encoded by a nucleic acid sequence selected from the group referenced in Table 17, rows 2-935, columns H and J, or
   said KwaB polypeptide is encoded by a nucleic acid sequence selected from the group referenced in Table 17, rows 2-935, columns L and N, or a combination thereof;
(m) for said Defense system Xa, said JetA polypeptide is encoded by a nucleic acid sequence selected from the group referenced in Table 11, rows 2-2322, columns J and L, or
   said JetB polypeptide is encoded by a nucleic acid sequence selected from the group referenced in Table 11, rows 2-2322, columns N and P, or
   said JetC polypeptide is encoded by a nucleic acid sequence selected from the group referenced in Table 11, rows 2-2322, columns R and T, or
   said JetD polypeptide is encoded by a nucleic acid sequence selected from the group referenced in Table 11, rows 2-2322, columns V and X, or
   a combination thereof;
(n) for said Defense system Xb, said JetAⁱⁱ polypeptide is encoded by a nucleic acid sequence selected from the group referenced in Table 11, rows 2323-2844, columns J and L, or
   said JetBⁱⁱ polypeptide is encoded by a nucleic acid sequence selected from the group referenced in Table 11, rows 2323-2844, columns N and P, or
   said JetCⁱⁱ polypeptide is encoded by a nucleic acid sequence selected from the group referenced in Table 11, rows 2323-2844, columns R and T, or
   said JetDⁱⁱ polypeptide is encoded by a nucleic acid sequence selected from the group referenced in Table 11, rows 2323-2844, columns V and X, or
   a combination thereof;
(o) for said Defense system Xc, said JetAⁱⁱⁱ polypeptide is encoded by a nucleic acid sequence selected from the group referenced in Table 11, rows 2845-3174, columns J and L, or
   said JetBⁱⁱⁱ polypeptide is encoded by a nucleic acid sequence selected from the group referenced in Table 11, rows 2845-3174, columns N and P, or
   said JetCⁱⁱⁱ polypeptide is encoded by a nucleic acid sequence selected from the group referenced in Table 11, rows 2845-3174, columns R and T, or
   said JetDⁱⁱⁱ polypeptide is encoded by a nucleic acid sequence selected from the group referenced in Table 11, rows 2845-3174, columns V and X, or
   a combination thereof.

In another aspect, disclosed herein are defense systems encoded by a nucleic acid sequence, comprising at least one defense system (a)-(o), wherein said at least one defense system comprises
(a) a Defense System IV comprising at least two different polypeptide components selected from
   a HamA polypeptide comprising a pfam08878 domain or a DUF1837 domain or a combination thereof, or comprising an amino acid sequence having at least 80% identity to a sequence selected from the group referenced in Table 12 rows 2-1782 columns H and I; and
   a HamB polypeptide comprising COG1204 domain or a pfam00270 domain or a pfam00271 domain or any combination thereof, or comprising an amino acid sequence having at least 80% identity to a sequence selected from the group referenced in Table 12 rows 2-1782 columns L and M; or
(b) a Defense System Ia comprising at least two different polypeptide components selected from
   a ZorA polypeptide comprising a pfam01618 domain, or comprising an amino acid sequence having at least 80% identity to a sequence selected from the group referenced in Table 8 rows 2-1830 columns J and K;
   a ZorB polypeptide comprising a pfam13677 domain or a pfam00691 domain or a combination thereof, or comprising an amino acid sequence having at least 80% identity to a sequence selected from the group referenced in Table 8 rows 2-1830 columns N and O;
   a ZorC polypeptide comprising a pfam15611 domain, or comprising an amino acid sequence having at least 80% identity to a sequence selected from the group referenced in Table 8 rows 2-1174 columns R and S; and
   a ZorD polypeptide comprising a pfam00176 domain or a pfam00271 domain or a combination thereof, or comprising an amino acid sequence having at least 80% identity to a sequence selected from the group referenced in Table 8 rows 2-1174 columns V and W;
      or
(c) a Defense System Ib comprising a ZorE polypeptide comprising a pfam01844 domain or a COG3183 domain or a combination thereof, or comprising an amino acid sequence having at least 80% identity to a sequence selected from the group referenced in Table 8 rows 1175-1830 columns Z and AA; and at least one other polypeptide component selected from
   a ZorA polypeptide comprising a pfam01618 domain, or comprising an amino acid sequence having at least 80% identity to a sequence selected from the group referenced in Table 8 rows 2-1830 columns J and K; and
   a ZorB polypeptide comprising a pfam13677 domain or a pfam00691 domain or a combination thereof, or comprising an amino acid sequence having at least 80% identity to a sequence selected from the group referenced in Table 8 rows 2-1830 columns N and O; or
(d) a Defense System II comprising at least two different polypeptide components selected from
   a ThsA polypeptide comprising a pfam13289 domain or a pfam14519 domain or a combination thereof, or comprising an amino acid sequence having at least 80% identity to a sequence selected from the group referenced in Table 9 rows 2-2100 columns I and J; and
   a ThsB polypeptide comprising a pfam13676 domain or a pfam08937 domain or a pfam08357 domain or any combination thereof, or comprising an amino acid sequence having at least 80% identity to a sequence selected from the group referenced in Table 9 rows 2-2100 columns M and N, Q and R, U and V, Y and Z, and AC and AD; or
(e) a Defense System IIIa comprising at least two different polypeptide components selected from
   a DruA polypeptide comprising a DUF4338 domain or a pfam14236 domain or a combination thereof, or comprising an amino acid sequence having at least 80% identity to a sequence selected from the group referenced in Table 10 rows 2-123 columns J and K, or a combination thereof;
   a DruB polypeptide comprising an amino acid sequence having at least 80% identity to a sequence selected from the group referenced in Table 10 rows 2-123 columns N and O;
   a DruC polypeptide comprising an amino acid sequence having at least 80% identity to a sequence selected from the group referenced in Table 10 rows 2-123 columns R and S;
   a DruD polypeptide comprising an amino acid sequence having at least 80% identity to a sequence selected from the group referenced in Table 10 rows 2-123 columns V and W; and
   a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA; or
(f) a Defense System IIIb comprising at least two different polypeptide components selected from
   a DruM polypeptide comprising a pfam00145 domain or a COG0270 domain or a combination thereof, or comprising an amino acid sequence having at least 80% identity to a sequence selected from the group referenced in Table 10 rows 124-295 columns AD and AE;
   a DruF polypeptide comprising an amino acid sequence having at least 80% identity to a sequence selected from the group referenced in Table 10 rows 124-295 columns AH and AI;
   a DruG polypeptide comprising an amino acid sequence having at least 80% identity to a sequence selected from the group referenced in Table 10 rows 124-295 columns AL and AM; and
   a DruE polypeptide comprising a pfam00270 domain or a pfam00271 domain or a pfam09369 domain or a DUF1998 domain, or any combination thereof, or comprising an amino acid sequence having at least 80% identity to a sequence selected from the group referenced in Table 10 rows 2-1343 columns Z and AA; or
(g) a Defense System IIIc comprising at least two different polypeptide components selected from
   a DruH polypeptide comprising an amino acid sequence having at least 80% identity to a sequence selected from the group referenced in Table 10 rows 296-1343 columns AP and AQ; and
   a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA; or
(h) a Defense System V comprising a SduA polypeptide comprising a pfam14082 domain or a pfam01939 domain or a combination thereof, or comprising an amino acid sequence having at least 80% identity to a sequence selected from the group referenced in Table 13 rows 2-1247 columns H and I; or
(i) a Defense System VI comprising at least two different polypeptide components selected from
   a GajA polypeptide comprising a pfam13175 domain or a COG3593 domain or a combination thereof, or comprising an amino acid sequence having at least 80% identity to a sequence selected from the group referenced in Table 14 rows 2-4599 columns H and I; and
   a GajB polypeptide comprising a pfam04257 domain or a pfam00580 domain or a pfam13361 domain or any combination thereof, or comprising an amino acid sequence having at least 80% identity to a sequence selected from the group referenced in Table 14 rows 2-4599 columns L and M; or
(j) a Defense System VII comprising at least two different polypeptide components selected from
   a PtuA polypeptide comprising a pfam13304 domain or a pfam02463 domain or a combination thereof, or comprising an amino acid sequence having at least 80% identity to a sequence selected from the group referenced in Table 15 rows 2-12507 columns H and I; and
   a PtuB polypeptide comprising pfam13395 domain or a pfam 01844 domain or a combination thereof, or comprising an amino acid sequence having at least 80% identity to a sequence selected from the group referenced in Table 15 rows 2-2507 columns L and M; or
(k) a Defense System VIII comprising at least two different polypeptide components selected from
   a LmuA polypeptide comprising a pfam14130 domain or a DUF4297 domain or a combination thereof, or comprising an amino acid sequence having at least 80% identity to a sequence selected from the group referenced in Table 16 rows 2-698 columns H and I; and
   a LmuB polypeptide comprising a pfam02463 domain, or comprising an amino acid sequence having at least 80% identity to a sequence selected from the group referenced in Table 16 rows 2-698 columns L and M; or
(l) a Defense System IX comprising at least two different polypeptide components selected from
   a KwaA polypeptide comprising an amino acid sequence having at least 80% identity to a sequence selected from the group referenced in Table 17 rows 2-935 columns H and I; and
   a KwaB polypeptide comprising a pfam16162 domain or a DUF4868 domain or a combination thereof, or comprising an amino acid sequence having at least 80% identity to a sequence selected from the group referenced in Table 17 rows 2-935 columns L and M; or
(m) a Defense System Xa comprising at least two different polypeptide components selected from
   a JetA polypeptide comprising a pfam11855 domain or a DUF3375 domain or a combination thereof, or comprising an amino acid sequence having at least 80% identity to a sequence selected from the group referenced in Table 11 rows 2-2322 columns J and K;
   a JetB polypeptide comprising a pfam13835 domain or a DUF4194 domain or a combination thereof, or comprising an amino acid sequence having at least 80% identity to a sequence selected from the group referenced in Table 11 rows 2-2322 columns N and O;
   a JetC polypeptide comprising a pfam13555 domain or a pfam13558 domain or a COG4913 domain or any combination thereof, or comprising an amino acid sequence having at least 80% identity to a sequence selected from the group referenced in Table 11 rows 2-2322 columns R and S; and
   a JetD polypeptide comprising a pfam11795 domain or a DUF3322 domain or a pfam09983 domain or a DUF2220 domain or a COG4924 domain or any combination thereof, or comprising an amino acid sequence having at least 80% identity to a sequence selected from the group referenced in Table 11 rows 2-2322 columns V and W; or
(n) a Defense System Xb comprising at least two different polypeptide components selected from
   a JetAⁱⁱ polypeptide comprising a pfam09660 domain or a DUF2397 domain or a combination thereof, or comprising an amino acid sequence having at least 80% identity to a sequence selected from the group referenced in Table 11 rows 2323-2844 columns J and K;
   a JetBⁱⁱ polypeptide comprising a pfam09661 domain or a DUF2398 domain or a combination thereof, or comprising an amino acid sequence having at least 80% identity to a sequence selected from the group referenced in Table 11 rows 2323-2844 columns N and O;
   a JetCⁱⁱ polypeptide comprising a pfam13558 domain comprising an amino acid sequence having at least 80% identity to a sequence selected from the group referenced in Table 11 rows 2323-2844 columns R and S; and
   a JetDⁱⁱ polypeptide comprising a pfam11796 domain or a DUF3323 domain or a pfam09664 domain or a DUF2399 domain or a combination thereof, or comprising an amino acid sequence having at least 80% identity to a sequence selected from the group referenced in Table 11 rows 2323-2844 columns V and W; or
(o) a Defense System Xc comprising at least two different polypeptide components selected from
   a JetAⁱⁱⁱ polypeptide comprising an amino acid sequence having at least 80% identity to a sequence selected from the group referenced in Table 11 rows 2845-3174 columns J and K;
   a JetBⁱⁱⁱ polypeptide comprising an amino acid sequence having at least 80% identity to a sequence selected from the group referenced in Table 11 rows 2845-3174 columns N and O;
   a JetCⁱⁱⁱ polypeptide comprising a COG1196 domain or comprising an amino acid sequence having at least 80% identity to a sequence selected from the group referenced in Table 11 rows 2845-3174 columns R and S; and
   a JetDⁱⁱⁱ polypeptide comprising a pfam09983 domain or a pfam09664 domain or a DUF2220 or a DUF2399 domain or any combination thereof, or comprising an amino acid sequence having at least 80% identity to a sequence selected from the group referenced in Table 11 rows 2845-3174 columns V and W; or
(p) any combination of the Defense Systems Ia-Xc (a)-(o);
wherein said at least one defense system comprises a non-naturally occurring combination of components.

In a related aspect, a defense system disclosed wherein
(a) said Defense System IV comprises the HamA polypeptide and the HamB polypeptide; or
(b) said Defense System Ia comprises the ZorA polypeptide, the ZorB polypeptide, the ZorC polypeptide, and the ZorD polypeptide; or
(c) said Defense System Ib comprises the ZorA polypeptide, the ZorB polypeptide, and the ZorE polypeptide; or
(d) said Defense System II comprises the ThsA polypeptide, and the ThsB polypeptide; or
(e) said Defense System IIIa comprises the DruA polypeptide, the DruB polypeptide, the DruC polypeptide, the DruD polypeptide, and the DruE polypeptide; or
(f) said Defense System IIIb comprises the DruM polypeptide, the DruF polypeptide, the DruG polypeptide, and the DruE polypeptide; or
(g) said Defense System IIIc comprises the DruH polypeptide and the DruE polypeptide; or
(h) said Defense System V comprises the SduA polypeptide; or
(i) said Defense System VI comprises the GajA polypeptide and the GajB polypeptide; or
(j) said Defense System VII comprises the PtuA polypeptide and the PtuB polypeptide; or
(k) said Defense System VIII comprises the LmuA polypeptide and the LmuB polypeptide; or
(l) said Defense System IX comprises the KwaA polypeptide and the KwaB polypeptide; or
(m) said Defense System Xa comprises the JetA polypeptide, the JetB polypeptide, the JetC polypeptide, and the JetD polypeptide; or
(n) said Defense System Xb comprises the JetAⁱⁱ polypeptide, the JetBⁱⁱ polypeptide, the JetCⁱⁱ polypeptide, and the JetDⁱⁱ polypeptide; or
(o) said Defense System Xc comprises the JetAⁱⁱⁱ polypeptide, the JetBⁱⁱⁱ polypeptide, the JetCⁱⁱⁱ polypeptide, and the JetDⁱⁱⁱ polypeptide.

In another related aspect, at least one defense system disclosed herein or a combination thereof, provides a host cell with resistance to foreign nucleic acid invasion. In another related aspect, the resistance to foreign nucleic acid invasion comprises resistance to at least one phage infection, or resistance to plasmid transformation, or resistance to entry of a conjugative element, or any combination thereof.

In another aspect, disclosed herein is a nucleic acid construct encoding an at least one defense system (a)-(o), said nucleic acid construct comprising
(a) a nucleic acid construct encoding a Defense System IV comprising at least two different polypeptide components selected from
   a HamA polypeptide comprising a pfam08878 domain or a DUF1837 domain or a combination thereof, or comprising an amino acid sequence having at least 80% identity to a sequence selected from the group referenced in Table 12 rows 2-1782 columns H and I; and
   a HamB polypeptide comprising COG1204 domain or a pfam00270 domain or a pfam00271 domain or any combination thereof, or comprising an amino acid sequence having at least 80% identity to a sequence selected from the group referenced in Table 12 rows 2-1782 columns L and M; or
(b) a nucleic acid construct encoding a Defense System Ia comprising at least two different polypeptide components selected from
   a ZorA polypeptide comprising a pfam01618 domain, or comprising an amino acid sequence having at least 80% identity to a sequence selected from the group referenced in Table 8 rows 2-1830 columns J and K;
   a ZorB polypeptide comprising a pfam13677 domain or a pfam00691 domain or a combination thereof, or comprising an amino acid sequence having at least 80% identity to a sequence selected from the group referenced in Table 8 rows 2-1830 columns N and O;
   a ZorC polypeptide comprising a pfam15611 domain, or comprising an amino acid sequence having at least 80% identity to a sequence selected from the group referenced in Table 8 rows 2-1174 columns R and S; and
   a ZorD polypeptide comprising a pfam00176 domain or a pfam00271 domain or a combination thereof, or comprising an amino acid sequence having at least 80% identity to a sequence selected from the group referenced in Table 8 rows 2-1174 columns V and W;
   or
(c) a nucleic acid construct encoding a Defense System Ib comprising a ZorE polypeptide comprising a pfam01844 domain or a COG3183 domain or a combination thereof, or comprising an amino acid sequence having at least 80% identity to a sequence selected from the group referenced in Table 8 rows 1175-1830 columns Z and AA; and at least one other polypeptide component selected from
   a ZorA polypeptide comprising a pfam01618 domain, or comprising an amino acid sequence having at least 80% identity to a sequence selected from the group referenced in Table 8 rows 2-1830 columns J and K; and
   a ZorB polypeptide comprising a pfam13677 domain or a pfam00691 domain or a combination thereof, or comprising an amino acid sequence having at least 80% identity to a sequence selected from the group referenced in Table 8 rows 2-1830 columns N and O; or
(d) a nucleic acid construct encoding a Defense System II comprising at least two different polypeptide components selected from
   a ThsA polypeptide comprising a pfam13289 domain or a pfam14519 domain or a combination thereof, or comprising an amino acid sequence having at least 80% identity to a sequence selected from the group referenced in Table 9 rows 2-2100 columns I and J; and
   a ThsB polypeptide comprising a pfam13676 domain or a pfam08937 domain or a pfam08357 domain or any combination thereof, or comprising an amino acid sequence having at least 80% identity to a sequence selected from the group referenced in Table 9 rows 2-2100 columns M and N, Q and R, U and V, Y and Z, and AC and AD; or
(e) a nucleic acid construct encoding a Defense System IIIa comprising at least two different polypeptide components selected from
   a DruA polypeptide comprising a DUF4338 domain or a pfam14236 domain or a combination thereof, or comprising an amino acid sequence having at least 80% identity to a sequence selected from the group referenced in Table 10 rows 2-123 columns J and K, or a combination thereof;
   a DruB polypeptide comprising an amino acid sequence having at least 80% identity to a sequence selected from the group referenced in Table 10 rows 2-123 columns N and O;
   a DruC polypeptide comprising an amino acid sequence having at least 80% identity to a sequence selected from the group referenced in Table 10 rows 2-123 columns R and S;
   a DruD polypeptide comprising an amino acid sequence having at least 80% identity to a sequence selected from the group referenced in Table 10 rows 2-123 columns V and W; and
   a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA; or
(f) a nucleic acid construct encoding a Defense System IIIb comprising at least two different polypeptide components selected from
   a DruM polypeptide comprising a pfam00145 domain or a COG0270 domain or a combination thereof, or comprising an amino acid sequence having at least 80% identity to a sequence selected from the group referenced in Table 10 rows 124-295 columns AD and AE;
   a DruF polypeptide comprising an amino acid sequence having at least 80% identity to a sequence selected from the group referenced in Table 10 rows 124-295 columns AH and AI;
   a DruG polypeptide comprising an amino acid sequence having at least 80% identity to a sequence selected from the group referenced in Table 10 rows 124-295 columns AL and AM; and
   a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA; or
(g) a nucleic acid construct encoding a Defense System IIIc comprising at least two different polypeptide components selected from
   a DruH polypeptide comprising an amino acid sequence having at least 80% identity to a sequence selected from the group referenced in Table 10 rows 296-1343 columns AP and AQ; and
   a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA; or
(h) a nucleic acid construct encoding a Defense System V comprising a SduA polypeptide comprising a pfam14082 domain or a pfam01939 domain or a combination thereof, or comprising an amino acid sequence having at least 80% identity to a sequence selected from the group referenced in Table 13 rows 2-1247 columns H and I; or
(i) a nucleic acid construct encoding a Defense System VI comprising at least two different polypeptide components selected from
   a GajA polypeptide comprising a pfam13175 domain or a COG3593 domain or a combination thereof, or comprising an amino acid sequence having at least 80% identity to a sequence selected from the group referenced in Table 14 rows 2-4599 columns H and I; and
   a GajB polypeptide comprising a pfam04257 domain or a pfam00580 domain or a pfam13361 domain or any combination thereof, or comprising an amino acid sequence having at least 80% identity to a sequence selected from the group referenced in Table 14 rows 2-4599 columns L and M; or
(j) a nucleic acid construct encoding a Defense System VII comprising at least two different polypeptide components selected from
   a PtuA polypeptide comprising a pfam13304 domain or a pfam02463 domain or a combination thereof, or comprising an amino acid sequence having at least 80% identity to a sequence selected from the group referenced in Table 15 rows 2-12507 columns H and I; and
   a PtuB polypeptide comprising pfam13395 domain or a pfam 01844 domain or a combination thereof, or comprising an amino acid sequence having at least 80% identity to a sequence selected from the group referenced in Table 15 rows 2-2507 columns L and M; or
(k) a nucleic acid construct encoding a Defense System VIII comprising at least two different polypeptide components selected from
   a LmuA polypeptide comprising a pfam14130 domain or a DUF4297 domain or a combination thereof, or comprising an amino acid sequence having at least 80% identity to a sequence selected from the group referenced in Table 16 rows 2-698 columns H and I; and
   a LmuB polypeptide comprising a pfam02463 domain, or comprising an amino acid sequence having at least 80% identity to a sequence selected from the group referenced in Table 16 rows 2-698 columns L and M; or
(l) a nucleic acid construct encoding a Defense System IX comprising at least two different polypeptide components selected from
   a KwaA polypeptide comprising an amino acid sequence having at least 80% identity to a sequence selected from the group referenced in Table 17 rows 2-935 columns H and I; and
   a KwaB polypeptide comprising a pfam16162 domain or a DUF4868 domain or a combination thereof, or comprising an amino acid sequence having at least 80% identity to a sequence selected from the group referenced in Table 17 rows 2-935 columns L and M; or
(m) a nucleic acid construct encoding a Defense System Xa comprising at least two different polypeptide components selected from
   a JetA polypeptide comprising a pfam11855 domain or a DUF3375 domain or a combination thereof, or comprising an amino acid sequence having at least 80% identity to a sequence selected from the group referenced in Table 11 rows 2-2322 columns J and K;
   a JetB polypeptide comprising a pfam13835 domain or a DUF4194 domain or a combination thereof, or comprising an amino acid sequence having at least 80% identity to a sequence selected from the group referenced in Table 11 rows 2-2322 columns N and O;
   a JetC polypeptide comprising a pfam13555 domain or a pfam13558 domain or a COG4913 domain or any combination thereof, or comprising an amino acid sequence having at least 80% identity to a sequence selected from the group referenced in Table 11 rows 2-2322 columns R and S; and
   a JetD polypeptide comprising a pfam11795 domain or a DUF3322 domain or a pfam09983 domain or a DUF2220 domain or a COG4924 domain or any combination thereof, or comprising an amino acid sequence having at least 80% identity to a sequence selected from the group referenced in Table 11 rows 2-2322 columns V and W; or
(n) a nucleic acid construct encoding a Defense System Xb comprising at least two different polypeptide components selected from
   a JetAⁱⁱ polypeptide comprising a pfam09660 domain or a DUF2397 domain or a combination thereof, or comprising an amino acid sequence having at least 80% identity to a sequence selected from the group referenced in Table 11 rows 2323-2844 columns J and K;
   a JetBⁱⁱ polypeptide comprising a pfam09661 domain or a DUF2398 domain or a combination thereof, or comprising an amino acid sequence having at least 80% identity to a sequence selected from the group referenced in Table 11 rows 2323-2844 columns N and O;
   a JetCⁱⁱ polypeptide comprising a pfam13558 domain comprising an amino acid sequence having at least 80% identity to a sequence selected from the group referenced in Table 11 rows 2323-2844 columns R and S; and
   a JetDⁱⁱ polypeptide comprising a pfam11796 domain or a DUF3323 domain or a pfam09664 domain or a DUF2399 domain or a combination thereof, or comprising an amino acid sequence having at least 80% identity to a sequence selected from the group referenced in Table 11 rows 2323-2844 columns V and W; or
(o) a nucleic acid construct encoding a Defense System Xc comprising at least two different polypeptide components selected from
   a JetAⁱⁱⁱ polypeptide comprising an amino acid sequence having at least 80% identity to a sequence selected from the group referenced in Table 11 rows 2845-3174 columns J and K;
   a JetBⁱⁱⁱ polypeptide comprising an amino acid sequence having at least 80% identity to a sequence selected from the group referenced in Table 11 rows 2845-3174 columns N and O;
   a JetCⁱⁱⁱ polypeptide comprising a COG1196 domain or comprising an amino acid sequence having at least 80% identity to a sequence selected from the group referenced in Table 11 rows 2845-3174 columns R and S; and
   a JetDⁱⁱⁱ polypeptide comprising a pfam09983 domain or a pfam09664 domain or a DUF2220 or a DUF2399 domain or any combination thereof, or comprising an amino acid sequence having at least 80% identity to a sequence selected from the group referenced in Table 11 rows 2845-3174 columns V and W; or
(p) any combination of nucleic acid constructs encoding a Defense Systems Ia-Xc (a)-(o);
wherein each nucleic acid construct of each Defense System (a)-(o) further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof.

In a related aspect, the expressed defense system comprises
(a) a Defense System IV comprising the HamA polypeptide, and the HamB polypeptide; or
(b) a Defense System Ia comprising the ZorA polypeptide, the ZorB polypeptide, the ZorC polypeptide, and the ZorD polypeptide; or
(c) a Defense System Ib comprising the ZorA polypeptide, the ZorB polypeptide, and the ZorE polypeptide; or
(d) a Defense System II comprising the ThsA polypeptide, and the ThsB polypeptide; or
(e) a Defense System IIIa comprising the DruA polypeptide, the DruB polypeptide, the DruC polypeptide, the DruD polypeptide, and the DruE polypeptide; or
(f) a Defense System IIIb comprising the DruM polypeptide, the DruF polypeptide, the DruG polypeptide, and the DruE polypeptide; or
(g) a Defense System IIIc comprising the DruH polypeptide and the DruE polypeptide; or
(h) a Defense System V comprising the SduA polypeptide; or
(i) a Defense System VI comprising the GajA polypeptide and the GajB polypeptide; or
(j) a Defense System VII comprising the PtuA polypeptide and the PtuB polypeptide; or
(k) a Defense System VIII comprising the LmuA polypeptide and the LmuB polypeptide; or
(l) a Defense System IX comprising the KwaA polypeptide and the KwaB polypeptide; or
(m) a Defense System Xa comprising the JetA polypeptide, the JetB polypeptide, the JetC polypeptide, and the JetD polypeptide; or
(n) a Defense System Xb comprising the JetAⁱⁱ polypeptide, the JetBⁱⁱ polypeptide, the JetCⁱⁱ polypeptide, and the JetDⁱⁱ polypeptide; or
(o) a Defense System Xc comprising the JetAⁱⁱⁱ polypeptide, the JetBⁱⁱⁱ polypeptide, the JetCⁱⁱⁱ polypeptide, and the JetDⁱⁱⁱ polypeptide.

In another related aspect, the nucleic acid construct disclosed herein, wherein

(a) said Defense System IV is encoded by
a nucleic acid sequence selected from the group referenced in Table 12, rows 2-1782, columns G, P, and Q or the nucleic acid sequence set forth in SEQ ID NO: 4; or
a nucleic acid sequence encoding the at least two different polypeptide sequences as disclosed herein; or
a nucleic acid sequence encoding the HamA polypeptide and HamB polypeptide as disclosed herein;

(b) said Defense System Ia is encoded by
a nucleic acid sequence selected from the group referenced in Table 8 rows 2-1174 columns G, AD, and AE or the nucleic acid sequence set forth in SEQ ID NO: 14; or
a nucleic acid sequence encoding the at least two different polypeptide sequences as disclosed herein; or
a nucleic acid sequence encoding the ZorA polypeptide, the ZorB polypeptide, the ZorC polypeptide, and ZorD polypeptide as disclosed herein;

(c) said Defense System Ib is encoded by
a nucleic acid sequence selected from the group referenced in Table 8 rows 1175-1830 columns G, AD, and AE or set forth in SEQ ID NO: 15; or
a nucleic acid sequence encoding the at least two different polypeptide sequences as disclosed herein; or
   a nucleic acid sequence encoding the ZorA polypeptide, the ZorB polypeptide, and ZorE polypeptide as disclosed herein;

(d) said Defense System II is encoded by
a nucleic acid sequence selected from the group referenced in Table 9 rows 2-2100 columns G, AG, and AH or set forth in the nucleic acid sequence set forth in SEQ ID NO: 10 or SEQ ID NO: 11; or
a nucleic acid sequence encoding the at least two different polypeptide sequences as disclosed herein; or
a nucleic acid sequence encoding the ThsA polypeptide and the ThsB polypeptide as disclosed herein;

(e) said Defense System IIIa is encoded by
a nucleic acid sequence selected from the group referenced in Table 10 rows 2-123 columns G, AT, and AU or set forth in SEQ ID NO: 16; or
a nucleic acid sequence encoding the at least two different polypeptide sequences as disclosed herein; or
a nucleic acid sequence encoding the DruA polypeptide, the DruB polypeptide, the DruC, the DruD, and the DruE polypeptide as disclosed herein;

(f) said Defense System IIIb is encoded by
a nucleic acid sequence selected from the group referenced in Table 10 rows 124-295 columns G, AT, AU; or
a nucleic acid sequence encoding the at least two different polypeptide sequences as disclosed herein; or
a nucleic acid sequence encoding the DruM polypeptide, the DruF polypeptide, the DruG polypeptide, and the DruE polypeptide as disclosed herein;

(g) said Defense System IIIc is encoded by
a nucleic acid sequence selected from the group referenced in Table 10 rows 296-1343 columns G, AT, and AU; or
a nucleic acid sequence encoding the at least two different polypeptide sequences as disclosed herein; or
a nucleic acid sequence encoding the DruH polypeptide and the DruE polypeptide as disclosed herein;

(h) said Defense System V is encoded by
a nucleic acid sequence selected from the group referenced in Table 13; rows 2-1247, columns H and J; or
a nucleic acid sequence encoding the SduA polypeptide disclosed herein;

(i) said Defense System VI is encoded by
a nucleic acid sequence selected from the group referenced in Table 14, rows 2-4599, columns G, P, and Q or the sequences set forth in SEQ ID NO: 5 and SEQ ID NO: 6; or
a nucleic acid sequence encoding the at least two different polypeptide sequences as disclosed herein; or
a nucleic acid sequence encoding the GajA polypeptide and the GajB polypeptide as disclosed herein;

(j) said Defense System VII is encoded by
a nucleic acid sequence selected from the group referenced in Table 15, rows 2-2507, columns G, P, and Q or the sequences set forth in SEQ ID NO: 12 and SEQ ID NO: 13; or
a nucleic acid sequence encoding the at least two different polypeptide sequences as disclosed herein; or
a nucleic acid sequence encoding the Ptu polypeptide and the PtuB polypeptide as disclosed herein;

(k) said Defense System VIII is encoded by
a nucleic acid sequence selected from the group referenced in Table 16, rows 2-698, columns G, P, and Q or the sequences set forth in SEQ ID NO: 7 and SEQ ID NO: 8; or
a nucleic acid sequence encoding the at least two different polypeptide sequences as disclosed herein; or
a nucleic acid sequence encoding the LmuA polypeptide and LmuB polypeptide as disclosed herein;

(l) said Defense System IX is encoded by
a nucleic acid sequence selected from the group referenced in Table 17, rows 2-935, columns G, P, and Q or the sequence set forth in SEQ ID NO: 3; or
a nucleic acid sequence encoding the at least two different polypeptide sequences as disclosed herein; or
a nucleic acid sequence encoding the KwaA polypeptide and the KwaB polypeptide as disclosed herein;

(m) said Defense System Xa is encoded by
a nucleic acid sequence selected from the group referenced in Table 11, rows 2-2322, columns G, Z, and AA or the sequence set forth in SEQ ID NO: 17; or
a nucleic acid sequence encoding the at least two different polypeptide sequences as disclosed herein; or
a nucleic acid sequence encoding the JetA polypeptide, the JetB polypeptide, the JetC polypeptide, and the JetD polypeptide as disclosed herein;

(n) said Defense System Xb is encoded by
a nucleic acid sequence having at least 80% homology to a sequence selected from the group referenced in Table 11, rows 2323-2844, columns G, Z, and AA or the sequence set forth in SEQ ID NO: 18; or
a nucleic acid sequence encoding the at least two different polypeptide sequences as disclosed herein; or
a nucleic acid sequence encoding the JetAⁱⁱ polypeptide, the JetBⁱⁱ polypeptide, the JetCⁱⁱ polypeptide, and the JetDⁱⁱ polypeptide as disclosed herein;

(o) said Defense System Xc is encoded by
a nucleic acid sequence selected from the group referenced in Table 11, rows 2845-3174, columns G, Z, and AA or the sequence set forth in SEQ ID NO: 19; or
a nucleic acid sequence encoding the at least two different polypeptide sequences as disclosed herein; or
a nucleic acid sequence encoding the JetAⁱⁱⁱ polypeptide, the JetBⁱⁱⁱ polypeptide, the JetCⁱⁱⁱ polypeptide, and the JetDⁱⁱⁱ polypeptide as disclosed herein.

Further, in another related aspect, a nucleic acid construct disclosed herein comprises a nucleic acid construct wherein
(a) said HamA polypeptide is encoded by a nucleic acid sequence selected from the group referenced in Table 12, rows 2-1782, columns H and J, or
   said HamB polypeptide is encoded by a nucleic acid sequence selected from the group referenced in Table 12, rows 2-1782, columns L and N, or
   a combination thereof;
(b) said ZorA polypeptide is encoded by a nucleic acid sequence selected from the group referenced in Table 8 rows 2-1830 columns J and L,
   said ZorB polypeptide is encoded by a nucleic acid sequence selected from the group referenced in Table 8 rows 2-1830 columns N and P,
   said ZorC polypeptide is encoded by a nucleic acid sequence selected from the group referenced in Table 8 rows 2-1174 columns R and T, or
   said ZorD polypeptide is encoded by a nucleic acid sequence selected from the group referenced in Table 8 rows 2-1174 columns V and X, or
   a combination thereof;
(c) said ZorA polypeptide is encoded by a nucleic acid sequence selected from the group referenced in Table 8 rows 2-1830 columns J and L,
   said ZorB polypeptide is encoded by a nucleic acid sequence selected from the group Table 8 rows 2-1830 columns N and P,
   said ZorE polypeptide is encoded by a nucleic acid sequence selected from the group referenced in Table 8 rows 1175-1830 columns Z and AB, or
   a combination thereof;
(d) said ThsA polypeptide is encoded by a nucleic acid sequence selected from the group referenced in Table 9 rows 2-2100 columns I and K,
   said ThsB polypeptide is encoded by a nucleic acid sequence selected from the group referenced in Table 9 rows 2-2100 columns M and O, Q and S, U and W, Y and AA, and AC and AE, or
   a combination thereof;
(e) said DruA polypeptide is encoded by a nucleic acid sequence selected from the group referenced in Table 10 rows 2-123 columns J and L, or
   said DruB polypeptide is encoded by a nucleic acid sequence selected from the group referenced in Table 10 rows 2-123 columns N and P, or
   said DruC polypeptide is encoded by a nucleic acid sequence selected from the group referenced in Table 10 rows 2-123 columns R and T, or
   said DruD polypeptide is encoded by a nucleic acid sequence selected from the group referenced in Table 10 rows 2-123 columns V and X, or
   said DruE polypeptide is encoded by a nucleic acid sequence selected from the group referenced in Table 10 rows 2-1343 columns Z and AB, or
   a combination thereof;
(f) said DruM polypeptide is encoded by a nucleic acid sequence selected from the group referenced in Table 10 rows 124-295 columns AD and AF, or
   said DruF polypeptide is encoded by a nucleic acid sequence selected from the group referenced in Table 10 rows 124-295 columns AH and AJ, or
   said DruG polypeptide is encoded by a nucleic acid sequence selected from the group referenced in Table 10 rows 124-295 columns AL and AN, or
   said DruE polypeptide is encoded by a nucleic acid sequence selected from the group referenced in Table 10 rows 2-1343 columns Z and AB, or
   a combination thereof;
(g) said DruH polypeptide is encoded by a nucleic acid sequence selected from the group referenced in Table 10 rows 296-1343 columns AP and AR, or
   said DruE polypeptide is encoded by a nucleic acid sequence selected from the group referenced in Table 10 rows 2-1343 columns Z and AB, or
   a combination thereof;
(h) said SduA polypeptide is encoded by a nucleic acid sequence selected from the group referenced in Table 13; rows 2-1247, columns H and J;
(i) said GajA polypeptide is encoded by a nucleic acid sequence selected from the group referenced in Table 14, rows 2-4599, columns H and J, or
   said GajB polypeptide is encoded by a nucleic acid sequence selected from the group referenced in Table 14, rows 2-4599, columns L and N, or
   a combination thereof;
(j) said PtuA polypeptide is encoded by a nucleic acid sequence selected from the group referenced in Table 15, rows 2-2507, columns H and J, or
   said PtuB polypeptide is encoded by a nucleic acid sequence selected from the group referenced in Table 15, rows 2-2507, columns L and N, or
   a combination thereof;
(k) said LmuA polypeptide is encoded by a nucleic acid sequence selected from the group referenced in Table 16, rows 2-698, columns H and J, or
   said LmuB polypeptide is encoded by a nucleic acid sequence selected from the group referenced in Table 16, rows 2-698, columns L and N, or
   a combination thereof;
(l) said KwaA polypeptide is encoded by a nucleic acid sequence selected from the group referenced in Table 17, rows 2-935, columns H and J, or
   said KwaB polypeptide is encoded by a nucleic acid sequence selected from the group referenced in Table 17, rows 2-935, columns L and N, or
   a combination thereof;
(m) said JetA polypeptide is encoded by a nucleic acid sequence selected from the group referenced in Table 11, rows 2-2322, columns J and L, or
   said JetB polypeptide is encoded by a nucleic acid sequence selected from the group referenced in Table 11, rows 2-2322, columns N and P, or
   said JetC polypeptide is encoded by a nucleic acid sequence selected from the group referenced in Table 11, rows 2-2322, columns R and T, or
   said JetD polypeptide is encoded by a nucleic acid sequence selected from the group referenced in Table 11, rows 2-2322, columns V and X, or
   a combination thereof;
(n) said JetAⁱⁱ polypeptide is encoded by a nucleic acid sequence selected from the group referenced in Table 11, rows 2323-2844, columns J and L, or
   said JetBⁱⁱ polypeptide is encoded by a nucleic acid sequence selected from the group referenced in Table 11, rows 2323-2844, columns N and P, or
   said JetCⁱⁱ polypeptide is encoded by a nucleic acid sequence selected from the group referenced in Table 11, rows 2323-2844, columns R and T, or
   said JetDⁱⁱ polypeptide is encoded by a nucleic acid sequence selected from the group referenced in Table 11, rows 2323-2844, columns V and X, or
   a combination thereof;
(o) said JetAⁱⁱⁱ polypeptide is encoded by a nucleic acid sequence selected from the group referenced in Table 11, rows 2845-3174, column J and L, or
   said JetBⁱⁱⁱ polypeptide is encoded by a nucleic acid sequence selected from the group referenced in Table 11, rows 2845-3174, columns N and O, or
   said JetCⁱⁱⁱ polypeptide is encoded by a nucleic acid sequence selected from the group referenced in Table 11, rows 2845-3174, columns R and T, or
   said JetDⁱⁱⁱ polypeptide is encoded by a nucleic acid sequence selected from the group referenced in Table 11, rows 2845-3174, columns V and X, or
   a combination thereof.

In another related aspect, disclosed herein is a nucleic acid construct wherein expression of said defense system from the nucleic acid construct in a host cells provides the host cell with resistance to foreign nucleic acid invasion. In another related aspect, resistance to foreign nucleic acid invasion comprises resistance to at least one phage infection, or resistance to plasmid transformation, or resistance to entry of a conjugation element, or any combination thereof.

In another aspect, disclosed herein is a transmissible genetic element or an expression vector comprising a nucleic acid construct as disclosed herein.

In another aspect, disclosed herein is an isolated cell expressing a nucleic acid construct as disclosed herein. In a related aspect, the cell comprises a gram-positive bacterium or a gram-negative bacterium. In another related aspect, the cell comprises resistance to foreign nucleic acid invasion. In still another related aspect, the resistance to foreign nucleic acid invasion comprises resistance to at least one phage infection, or reduced plasmid transformation efficiency, or resistance to entry of a conjugation element, or a combination thereof.

In another aspect, disclosed herein is a food, food additive, feed, nutritional supplement, probiotic supplement, personal care product, health care product, and or a veterinary product comprising any defense system or combination of defense systems as disclosed herein, or the nucleic acid construct or combination of constructs as disclosed herein, or the transmissible genetic element as disclosed herein, or the isolated cell as disclosed herein.

In a related aspect, the defense system or combination of defense systems included in a food, food additive, feed, nutritional supplement, probiotic supplement, personal care product, health care product, and or a veterinary product disclosed herein, comprises a combination of Defense Systems selected from Defense Systems Ia-Xc (a)-(o).

In another aspect, disclosed herein is a method of preparing a food, a food additive, a feed, a nutritional supplements, a probiotic supplement, a personal care product, a health care product or a veterinary product, the method comprising adding to the food, food additive, feed, nutritional supplement, probiotic supplement, personal care product, health care product, or veterinary product at least one defense system as disclosed herein, or the nucleic acid construct as disclosed herein, or the transmissible genetic element disclosed herein, or the isolated cell disclosed herein.

In another aspect, disclosed herein is a method for identifying a defense system in a prokaryotic cell, comprising the steps of:
(a) selecting, in-silico, a family of genes that are preferentially located in close proximity to a known defense-related gene family present in a plurality of prokaryotic genomes; and
(b) analyzing, in-silico, the DNA upstream and downstream of said gene; and
(c) identifying, in-silico, a cassette of genes within the upstream and downstream DNA analyzed in step (b) comprising said gene of step (a), wherein said cassette of genes has conserved synteny in said plurality of prokaryotic genomes.

In a related aspect, in a method of identifying a defense system, the gene of step (a), located in close proximity to a known defense related gene family, is within 10 genes upstream and or 10 genes downstream of the known defense related gene family.

In another aspect, disclosed herein is a method gene editing, said method comprising contacting a nucleic acid sequence comprising a gene to be edited with at least one polypeptide component of a defense system, wherein said defense system component comprises a component selected from the components of the defense system disclosed herein, wherein said method results in the gene being edited.

### BRIEF DESCRIPTION OF THE DRAWINGS

The subject matter disclosed herein is particularly pointed out and distinctly claimed in the concluding portion of the specification. The defense systems described, however, both as to organization and method of operation, together with objects, features, and advantages thereof, may best be understood by reference to the following detailed description when read with the accompanying drawings in which:

FIGS. 1A-1C presents the strategy and initial results for the discovery of new anti-phage defense systems in defense islands. FIG. 1A illustrates the computational analysis employed for each pfam found to be enriched in defense islands. Pfams that are enriched in the vicinity of known defense genes are identified, and their neighboring genes are clustered based on sequence homology to identify conserved cassettes that represent putative defense systems. FIG. 1B presents a bar graph showing the tendency of protein families to occur next to defense genes. The genomic neighborhood for each member gene in each pfam is examined, and the fraction of member genes occurring in the vicinity (10 genes on each side) of one or more known defense genes is recorded. A set of 123 pfams known to participate in anti-phage defense (“positive set”) are represented in pink; the remaining 13,960 pfams analyzed in this study are shown in blue. FIG. 1C presents the neighborhood variability score for analyzed pfams. Scores represent the fraction of pfams members occurring in different defense neighborhoods out of total occurrences of pfam members. (Pink, the 123 positive pfams; blue, a set of 576 pfams that passed the 65% threshold for fraction of members occurring with defense genes in proximity.)

FIGS. 2A and 2B present the distribution of predicted functions for pfams genomically associated with known defense genes. FIG. 2A presents the pfams retrieved from prediction cycle #1. FIG. 2B presents the pfams retrieved from prediction cycle #2.

FIGS. 3A-3H show the flowchart and experimentally verified defense systems. (FIG. 3A) Flowchart of the experimental verification strategy. (FIG. 3B) Active defense systems cloned into B. subtilis. (FIG. 3C) Active defense systems cloned into E. coli. For FIGS. 3B and 3C, the fold protection was measured using serial dilution plaque assays, comparing the system-containing strain to a control strain that lacks the system and has an empty vector instead. Data represents average of 3 replicates (FIGS. 3E and 3F). Numbers below phage names represent phage genome size. On the right of FIGS. 3B and 3C, gene organization of the defense systems, with identified domains indicated (DUF, domain of unknown function). Gene sizes are drawn to scale, scale bar represents 400 amino acids. FIG. 3D presents the initial data demonstrating that the ZORYA type I system provides protection against phages as evaluated by serial dilution plaque assay. E. coli MG1655 strain (control) and E. coli MG1655 strain (control) transfected with the ZORYA system I of ZORYA locus of Escherichia coli (E. coli) E24377A and challenged with the indicated phages (phage T4, phage T7, phage lambda vir t [an obligatory lytic mutant of phage lambda], SECphi27, SECphi17, and SECphi18). Columns represent serial 10-fold dilutions of the initial phage stock concentration. The leftmost column represents 10⁻² (100 fold) dilution, the second column from the right represents 10⁻³ (1000 fold dilution), and so on until the leftmost column that represents 10 million fold dilution. FIG. 3E presents the efficiency of plating (EOP) of Bacillus phages on defense systems cloned into B. subtilis BEST7003. Data represent PFU/ml values, average of 3 replicates; error bars are STD. When two representations of the same system are indicated (e.g. Thoeris I and Thoeris II), order of systems is as in FIG. 3B. FIG. 3F presents efficiency of plating (EOP) of coliphages on defense systems cloned into E. coli MG1655. Data represent PFU/ml values, average of 3 replicates; error bars are STD. Order of defense systems in each graph is as in FIG. 3C. FIG. 3G presents efficiency of plating (EOP) of Bacillus phage SpBeta on defense systems mutated in individual genes. Data represent PFU/ml values of SpBeta phage infecting WT and mutated systems. Average of 3 replicates; error bars are STD. Gene deletion results for systems Zorya, Thoeris, Wadjet and Druantia appear in FIGS. 4, 5, 7, and 6, respectively. Gene deletion attempts for the Kiwa system were not successful. FIG. 3H presents distribution of defense systems in sequenced microbial genomes. For each system, phyla in which instances of the system were detected are indicated. Top left pie chart represents the entire set of microbial genomes analyzed, and is shown in order to provide a reference for the phylogenetic distribution of the species whose genomes were sequenced. Data on individual instances of each system are found in Tables 4 and 8-18.

FIGS. 4A-4J present the identified members of the Type I and Type II ZORYA defense systems and analysis thereof. FIG. 4A shows schematic representative instances of the Type I ZORYA system and their defense island context. In the last four embodiments, genes known to be involved in defense are orange. Mobilome genes are in dark grey. RM—restriction modification; TA—toxin-antitoxin; Abi—abortive infection; Wadjet and DISARM are other defense systems identified herein. FIG. 4B presents the domain organization of the Type I ZORYA system comprising zorA (A), zorB (B), zorC (C) and zorD (D). FIG. 4C presents a schematic of the ZORYA type I defense system locus of Escherichia coli (E. coli) E24377A used for cloning. Numbers represent position on the E. coli E24377A genome. Specifically, nucleotides 298890-307639 of the reverse strand were cloned into the E. coli MG1655 genome (SEQ ID NO: 14). FIG. 4D shows multiple schematic representative instances of the Type II ZORYA system and their defense island context. Genes known to be involved in defense are orange. Mobilome genes are in dark grey. RM—restriction modification; TA—toxin-antitoxin; Druantia is another defense systems identified herein. FIG. 4E presents the domain organization of the Type II ZORYA system comprising zorA (A), zorB (B), and zorE (E). Domain organization of zorA and zorB are as in FIG. 4B. FIG. 4F presents a model of the flagellum base. The position of the MotAB complex is indicated. FIG. 4G presents percent phage infection of Zorya-containing cells in solid cultures. FIG. 4H presents optical density (O.D.) versus time post infection of phage infected Zorya Defense System Ia-containing bacteria in liquid cultures. FIG. 4I presents optical density (O.D.) versus time post infection of phage infected Zorya Defense System Ib-containing bacteria in liquid cultures. FIG. 4J presents the efficiency of plating (EOP) of phage SECphi27 infecting WT Type I ZORA, deletion strains, and strains with point mutations. Data represent PFU/ml, average of 3 replicates with error bars representing STD. ZorA:T147A/S184A and ZorB:D26N are predicted to abolish proton flux; ZorC:E400A/H443A are mutations in two conserved residues in pfam15611 (“EH domain”) whose function is unknown; ZorD:D730A/E731A are mutations in the Walker B motif, predicted to abolish ATP hydrolysis.

FIGS. 5A-5E present the identified members of the Thoeris system and analysis thereof. FIG. 5A shows schematic representative instances of the Thoeris (SIR2-TIR) system and their defense island context. In each row of the top set of systems identified, the red gene is the thsA gene (Sir2-domain; pfam13289) gene; the immediately downstream gene is the thsB gene (TIR domain; pfam08937 or pfam13676) gene. In the last four systems presented, the Thoeris genes thsA (containing NAD-binding domain) and thsB (TIR domain) are marked dark and light green, respectively. Genes known to be involved in defense are orange. Mobilome genes are in dark grey. RM, restriction-modification; TA, toxin-antitoxin; Abi, abortive infection. FIG. 5B presents schematic representations of two Thoeris systems identified and analyzed in this study, shown to protect against myophages. Locus tag accessions are indicated for the individual genes. FIG. 5C presents the efficiency of plating (EOP) results of phage SBSphiJ infection with WT and mutated versions of the B. amyloliquefaciens Y2 Thoeris (FIG. 5B top set) or B. cereus MSX-D12 Thoeris (FIG. 5B bottom set) cloned into B. subtilis BEST7003. Average of 3 replicates, error bars represent STD. FIG. 5D shows the initial results demonstrating that the Thoeris system (SIR2-TIR system) provides protection against phages as evaluated by plaque assay. Phage used were SBSphiC, SBSphiJ and SPO1. FIG. 5E presents predictions of the active site in ThsB polypeptide. Shown is an alignment of the ThsB gene from Bacillus amyloliquefaciens Y2 to SARM1 and MliB. Numbers in the alignment represent position in the protein sequence. Positions of verified active sites for MliB and SARM1, and the predicted active site of ThsB, are indicated.

FIGS. 6A-6C present the identified members of the Druantia system and analysis thereof. FIG. 6A presents representative instances of Druantia types I, II and III and their defense island context. The system is characterized by a gene encoding a large (1800aa-2100aa) polypeptide with a DUF1998 domain (druE), preceded by 3, 2, or 1 genes encoding hypothetical proteins with no domain annotation, for Types I, II and III, respectively. In some Type I cases, a gene encoding a polypeptide with a pfam14236 (DUF4338) domain (druA gene; DruA polypeptide) is associated with the system. Type II systems are typically associated with a cytosine methylase gene (DruM). Genes known to be involved in defense are orange. Mobilome genes are in dark grey. RM, restriction-modification; TA, toxin-antitoxin; Zorya, a defense system identified in this study. FIG. 6B presents the Druantia systems that was cloned (Example 6) and experimentally shown to protect against coliphages. Locus tag accessions are indicated for the individual genes. FIG. 6C presents data showing gene deletions render the Druantia defense system inactive. DruA deletion was not tested due to technical reasons. Data represent EOP, average of 3 replicates; error bars are STD.

FIG. 7A-7C present the identified members of the Wadjet system and analysis thereof. The Wadjet system was shown to provide protection against plasmid transformation in B. subtilis. (FIG. 7A) Representative instances of the Wadjet system and their defense island context. Genes known to be involved in defense are orange. RM, restriction-modification; TA, toxin-antitoxin; Abi, abortive infection. (FIG. 7B) Domain organization of the three types of Wadjet. (FIG. 7C) Wadjet reduces plasmid transformation efficiency in B. subtilis. Wadjet systems were taken from B. cereus Q1 (Type I), Bacillus vireti LMG 21834 (Type II) and Bacillus thuringiensis serovar finitimus YBT-020 (Type III) (Table 4) and cloned into B. subtilis BEST7003. Gene deletions and point mutations are of the B. cereus Q1 Type I Wadjet. Transformation efficiency of plasmid pHCMC05 into Wadjet-containing strains is presented as a percentage of the transformation efficiency to B. subtilis BEST7003 carrying an empty vector instead of the Wadjet system. Average of 3 replicates; error bars represent STD.

### DETAILED DESCRIPTION

In the following detailed description, numerous specific details are set forth in order to provide a thorough understanding of the defense systems disclosed herein, including cells comprising these systems, methods of producing cells comprising these systems, methods of identifying the defense systems, and products, for example but not limited to food products or probiotic supplements, that may comprise these the defense systems. In some instances, well-known methods, procedures, and components have not been described in detail so as not to obscure the present disclosure.

The evolutionary pressure imposed by phage predation on bacteria has resulted in the development of both anti-phage bacterial resistance systems and counter-resistance mechanisms developed by phages. Harnessing the anti-phage bacterial mechanisms to improve industrial processes by e.g. curtailing the propagation and evolution of phages in fermentation vats in the manufacturing of food products, commodity chemicals, and biotechnology products is a standing goal in biological research. On the counter arm, properly formulated and applied phages or their defense mechanisms have sufficient potential to cure bacterial infections.

In some embodiments, disclosed herein are the defense systems, including but not limited to anti-phage defense systems, methods for producing and using such defense systems, and methods of countering such defense systems. In some embodiments, disclosed herein are defense systems that provide a host cell with resistance to foreign nucleic acid invasion. In some embodiments, a defense system described herein, provides the host cell with resistance to a foreign nucleic acid invasion, wherein the foreign nucleic acid invasion comprises resistance to at least one phage infection, or resistance to plasmid transformation, or a combination of resistance to at least one phage infection and resistance to plasmid transformation. In some embodiments, it is the combination of defense systems that provides a host cell with resistance to a foreign nucleic acid invasion.

Taken together, the disclosure presented herein of anti-phage defense systems or functional portions thereof may in some embodiments be used for conferring phage resistance in microbial cells. Bacteria comprising these anti-phage defense systems can be utilized for example in the dairy industry, where phages cause serious annual losses, as well as in other industries that rely on large-scale bacterial fermentation for biotechnological production. Alternatively, an agent able to antagonize a defense system may in some embodiments can be used as an antibiotic or in conjunction with known antibiotics.

In addition, embodiments disclosed herein include methods of use of defense systems for gene editing, for example but not limited to, for cloning and cutting a nucleic acid sequence leading to specific deletion of a gene, repair of a gene, or replacement of a gene.

Genetic Systems that Defend Against Foreign Nucleic Acid (DNA and RNA)

In some embodiments, disclosed herein is a genetic system that defends a host cell against foreign nucleic acid invasion. One skilled in the art would appreciate that defense against a foreign nucleic acid invasion may encompass, defending against entry of a foreign nucleic acid into the host cell, as well as, defending against the actions of a foreign nucleic acid that has entered the host cell. In some embodiments, defense against a foreign nucleic acid invasion comprises defense from phage infection. In some embodiments, defense against a foreign nucleic acid invasion comprises defense from plasmid transformation. In some embodiments, defense against a foreign nucleic acid invasion comprises defense against entry of a conjugative element. In some embodiments, defense against a foreign nucleic acid invasion comprises defense against any combination of phage infection, plasmid transformation, and entry of a conjugative element.

In some embodiments a foreign nucleic acid comprises a foreign DNA. In some embodiments a foreign nucleic acid comprises a foreign RNA.

In some embodiments, disclosed herein is a genetic system that defends against foreign DNA. (Tables 4, and 6-17) The skilled artisan would appreciate that as used herein the term “defense system” encompasses genetic systems that defend against foreign nucleic acid elements, including foreign DNA elements and foreign RNA elements.

In some embodiments, a defense system described herein functions as an anti-phage defense system, wherein the system provides a microbial cell resistance to phage infection. In some embodiments, a defense system described herein functions as an anti-phage defense system, wherein the system provides bacteria resistance to phage infection. In some embodiments, the defense system functions as an anti-plasmid transformation defense system, wherein the system reduces the transformation of a host cell by plasmid vectors. In some embodiments, the defense system functions as an anti-plasmid transformation defense system, wherein the system reduces the transformation of a microbial cell by plasmid vectors. In some embodiments, the defense system functions as an anti-plasmid transformation defense system, wherein the system reduces the transformation of bacteria by plasmid vectors.

The skilled artisan would appreciate that a defense system may in some embodiments, be introduced into a microbial cell in which such a defense system is not present. In some embodiments, a defense system may be introduced into a bacterium cell in which such a defense system is not present. In some embodiments, a defense system may be introduced into a microbial cell in which such a defense system is not functional. In some embodiments, a defense system may be introduced into a bacterium cell in which such a defense system is not functional. In some embodiments, a defense system may be introduced into a microbial cell in which such a defense system is not expressed. In some embodiments, a defense system may be introduced into a bacterium cell in which such a defense system is not expressed.

In some embodiments, introduction of a defense system into a microbial cell in which such a defense system is not present, provides the microbial cell resistance to foreign nucleic acid invasion. In some embodiments, integration of a defense system into the genome of a microbial cell in which such the defense system is not present, provides the microbial cell resistance to foreign nucleic acid invasion. In some embodiments, integration of functional components of a defense system into the genome of a microbial cell in which such the defense system is not present, provides the microbial cell resistance to foreign nucleic acid invasion. In some embodiments, integration of at least one functional component of a defense system into the genome of a microbial cell in which such the defense system is not present, provides the microbial cell resistance to foreign nucleic acid invasion.

In some embodiments, introduction of a defense system into a microbial cell in which such a defense system is not present, provides the microbial cell resistance to phage infection. In some embodiments, integration of a defense system into the genome of a microbial cell in which such the defense system is not present, provides the microbial cell resistance to phage infection. In some embodiments, integration of functional components of a defense system into the genome of a microbial cell in which such the defense system is not present, provides the microbial cell resistance to phage infection. In some embodiments, integration of at least one functional component of a defense system into the genome of a microbial cell in which such the defense system is not present, provides the microbial cell resistance to phage infection.

In some embodiments, introduction of a defense system into a bacterium cell in which such a defense system is not present, provides the bacterium resistance to foreign nucleic acid invasion. In some embodiments, introduction of a defense system into a bacterium cell in which such a defense system is not present, provides the bacterium resistance to phage infection. In some embodiments, integration of a defense system into the genome of a bacterial cell in which such the defense system is not present, provides the bacterium cell resistance to phage infection. In some embodiments, integration of functional components of a defense system into the genome of a bacterium cell in which such the defense system is not present, provides the bacterium cell resistance to phage infection. In some embodiments, integration of at least one functional component of a defense system into the genome of a bacterium cell in which such the defense system is not present, provides the bacterial cell resistance to phage infection. Resistance to phage infection in bacteria may occur for example, but not limited to, abortive infection of a phage, blocking phage adsorption to the bacterium surface, acquired immunity, preventing phage lysogeny in bacteria, causing degradation of phage genome in bacteria, inhibition of phage replication, and inhibition of phage genome replication. not causing an abortive infection;

In some embodiments, a defense system disclosed herein is not present in a microbial cell species. In some embodiments, a defense system disclosed herein is not present in a bacterial cell species.

In some embodiments, a defense system disclosed herein is not functional in a microbial cell species. In some embodiments, a defense system disclosed herein is not functional in a bacterial cell species. Thus, in some embodiments, introduction of a defense system into a microbial cell lacking a functional version of the defense system disclosed herein, provides resistance to foreign nucleic acid invasion. In some embodiments, introduction of a defense system into a microbial cell lacking a functional version of the defense system disclosed herein, provides resistance to phage infection.

In some embodiments, introduction of a defense system into a microbial cell in which such a defense system is not present, provides the microbial cell resistance to plasmid transformation. In some embodiments, integration of a defense system into the genome of a microbial cell in which such the defense system is not present, provides the microbial cell resistance to plasmid transformation. In some embodiments, integration of functional components of a defense system into the genome of a microbial cell in which such the defense system is not present, provides the microbial cell resistance to plasmid transformation. In some embodiments, integration of at least one functional component of a defense system into the genome of a microbial cell in which such the defense system is not present, provides the microbial cell resistance to plasmid transformation.

In some embodiments, introduction of a defense system into a bacterium cell in which such a defense system is not present, provides the bacterium resistance to plasmid transformation. In some embodiments, integration of a defense system into the genome of a bacterial cell in which such the defense system is not present, provides the bacterium cell resistance to plasmid transformation. In some embodiments, integration of functional components of a defense system into the genome of a bacterium cell in which such the defense system is not present, provides the bacterium cell resistance to plasmid transformation. In some embodiments, integration of at least one functional component of a defense system into the genome of a bacterium cell in which such the defense system is not present, provides the bacterial cell resistance to plasmid transformation. Resistance to plasmid in bacteria may occur for example, but not limited to, reduced transformation efficiency, inhibition of plasmid establishment, and or inhibition of transformation.

In some embodiments, introduction of a defense system into a bacterium cell in which such a defense system is not present, provides the bacterium protection from conjugative elements. In some embodiments, integration of a defense system into the genome of a bacterial cell in which such the defense system is not present, provides the bacterium cell protection from conjugative elements. In some embodiments, integration of functional components of a defense system into the genome of a bacterium cell in which such the defense system is not present, provides the bacterium cell resistance to conjugation with another bacterium. In some embodiments, integration of at least one functional component of a defense system into the genome of a bacterium cell in which such the defense system is not present, provides the bacterial cell resistance to conjugation with another bacterium. The skilled artisan would appreciate that the term “conjugative elements” may encompass mobile genetic elements, plasmids, and transposons.

A skilled artisan would appreciate that a defense system includes but is not limited to a system that defends a cell from foreign nucleic acid. In some embodiments, a defense system includes but is not limited to a system that defends a cell from foreign DNA. In some embodiments, a defense system includes but is not limited to a system that defends a cell from foreign RNA. In some embodiments, a defense system includes but is not limited to an anti-plasmid system. In some embodiments, a defense system includes but is not limited to an anti-transposon system. In some embodiments, a defense system includes but is not limited to an anti-conjugation system. In some embodiments, a defense system includes but is not limited to an anti-phage system.

In some embodiments, the nucleic acid comprises a linear nucleic acid molecule. In some embodiments, the nucleic acid comprises circular nucleic acid molecule. In some embodiments, the nucleic acid comprises a single stranded nucleic acid molecule. In some embodiments, the nucleic acid comprises a double stranded nucleic acid molecule. The skilled artisan would appreciate that foreign nucleic acid may in some embodiments, encompass any nucleic acid molecule foreign to, for example, but not limited to a microbial cell, a bacterium cell, or an archaeal cell.

In some embodiments, a foreign nucleic acid comprises a foreign DNA or foreign RNA. In some embodiments, a foreign DNA or foreign RNA comprises a phage. In some embodiments, a foreign DNA or foreign RNA comprises a plasmid. In some embodiments, a foreign DNA or foreign RNA comprises a conjugative element. In some embodiments, a foreign DNA or foreign RNA comprises a mobile genetic element. In some embodiments, a foreign DNA or foreign RNA comprises a mobile genetic element.

In some embodiments, a defense system protects bacteria from phage infection. In some embodiments, a defense system protects bacteria from plasmid transformation. In some embodiments, a defense system protects bacteria from conjugative elements. In some embodiments, a defense system provides a bacteria resistance to phage infection. In some embodiments, a defense system provides a bacteria resistance to plasmid transformation. In some embodiments, a defense system provides a bacteria resistance to entry of conjugative elements.

Disclosed herein, is the identification of defense systems. In some embodiments, the components of a defense system are located in a gene cluster in a prokaryotic cell. The terms “gene cluster”, “cassette of genes”, “cassette”, and “components of a system”, may in some embodiments herein be used interchangeably having all the same meanings and qualities.

In some embodiments, each gene of a “cassette of genes” comprises a nucleic acid sequence encoding a polypeptide component of the defense system. In some embodiments, a “cassette of genes” comprises nucleic acid sequences encoding components of the defense system including open reading frames encoding defense system polypeptide components, regulatory sequences, and non-coding RNAs. A skilled artisan would appreciate that a “cassette of genes” may encompass an operon. In some embodiments, a cassette of genes comprises regulatory sequences. In some embodiments, a cassette of gene comprises non-coding RNAs.

In some embodiments, disclosed herein is a nucleic acid construct of a Defense System. In some embodiments, a nucleic acid construct of a Defense System comprises at least two nucleic acid constructs each expressing at least one of the polypeptide components of the Defense System. In some embodiments, disclosed herein is a nucleic acid construct of a Defense System comprising at least two nucleic acid constructs each expressing at least one functional polypeptide component of the Defense System.

In some embodiments, the nucleic acid construct comprises a plurality of constructs each expressing a single component of a Defense System described herein. In some embodiments, the nucleic acid construct comprises a plurality of constructs each expressing a single functional component of a Defense System described herein.

In some embodiments, a single nucleic acid construct encodes a number of polypeptide components of a Defense System described herein. In some embodiments, a single construct encodes a number of functional polypeptide components of a Defense System described herein.

A skilled artisan would recognize that the term “anti-phage activity” or “resistant to infection by at least one phage” encompasses an activity providing increased resistance of a host cell to infection by at least one phage in comparison to the host cell of the same species under the same developmental stage (e.g. culture state) which does not express the functional defense system. In some embodiments, a host cell may comprise a microbial cell. In some embodiments, a host comprises a bacterium. Anti-phage activity or resistance of a host cell to infection by at least one phage may be determined by, for example but not limited to, bacterial viability, phage lysogeny, phage genomic replication or phage genomic degradation, or a combination thereof.

A skilled artisan would appreciate that the term “abortive infection (Abi)” encompasses a controlled cell death of an infected bacterial cell which takes place prior to the production of phage progeny, thus protecting the culture from phage propagation. Methods of analyzing Abi include, but are not limited to cell survival assays using high multiplicity of infection, one step growth assays and determination of phage DNA replication by e.g. DNA sequencing and southern blot analysis as further described hereinbelow.

A skilled artisan would appreciate that the phrase “not affecting phage adsorption” encompasses a non-statistically significant difference in phage adsorption to bacteria expressing a functional defense system in comparison to bacteria of the same species under the same developmental stage (e.g. culture state) which does not express a functional defense system.

A skilled artisan would appreciate that the term “adsorption” encompasses the attachment to the host (e.g. bacteria) cell surface via plasma membrane proteins and glycoproteins. Methods of analyzing phage adsorption include, but are not limited to enumerating free phages in bacterial cultures infected with the phages immediately after phage addition and at early time points (e.g. 30 minutes) following phage addition as further described hereinbelow.

A skilled artisan would appreciate that the term “prevent” or “preventing” encompasses a decrease in activity (e.g. phage genomic replication, phage lysogeny, circularization of phage genome) in bacteria expressing a functional defense system in comparison to bacteria of the same species under the same developmental stage (e.g. culture state) which does not express a functional defense system. According to specific embodiments the decrease is at least 1.5 fold, at least 2 fold, at least 3 fold, at least 5 fold, at least 10 fold, or at least 20 fold as compared to same in the absence of the functional defense system.

A skilled artisan would appreciate that the term “resistance” to, for example, foreign nucleic acid invasion, encompasses a decrease in activity (e.g. phage genomic replication, phage lysogeny, circularization of phage genome) in bacteria expressing a functional defense system in comparison to bacteria of the same species under the same developmental stage (e.g. culture state) which does not express a functional defense system. According to specific embodiments the decrease provided by such resistance to foreign nucleic acid invasion is at least 1.5 fold, at least 2 fold, at least 3 fold, at least 5 fold, at least 10 fold, or at least 20 fold as compared to same in the absence of the functional defense system.

A skilled artisan would appreciate that the term “phage genomic replication” encompasses production of new copies of the phage genome which can be dsDNA or ssDNA. Methods of analyzing phage genomic replication are well known in the art, and in the Examples section which follows.

A skilled artisan would appreciate that the term “lysogeny” encompasses the incorporation of the phage genetic material inside the genome of the host (e.g. bacteria). Methods of analyzing phage lysogeny are well known in the art and include, but not limited to, DNA sequencing and PCR analysis. Typically, when a temperate phage infects a bacterium, its genetic material becomes circular before it incorporates into the bacterial genome. Circularization of phage genome can be analyzed by methods well known in the art including, but not limited to, PCR analysis as described e.g. in the Examples section which follows.

When referring to “degradation of phage genome” the meaning is the cleavage of the foreign phage genome by the host bacteria. Method of analyzing genomic degradation are well known in the art including, but not limited to, DNA sequencing and PCR analysis.

A skilled artisan would appreciate that the term “restriction modification system” may encompass a restriction entity having an activity of cleaving a genomic molecule (e.g. DNA)/DNA and a modification entity capable of protecting (e.g., by methylation) the host DNA from the cleavage by the restriction enzyme e.g. by methylating the host DNA. Analyzing restriction modification mode of action include, but is not limited to, evaluation of host specific methylation, presence of degraded foreign DNA and host cell death in the absence of the modification enzyme by methods described herein.

In some embodiments, a functional defense system leads to an abortive infection in bacteria expressing the defense system. In some embodiments, a functional defense system reduces affect phage adsorption to bacteria expressing the defense system. In some embodiments, a functional defense system prevents phage genomic replication in bacteria expressing the defense system. In some embodiments, a functional defense system prevents phage lysogeny in bacteria expressing the defense system. In some embodiments, a functional defense system prevents circularization of a phage genome in bacteria expressing the defense system. In some embodiments, a functional defense system leads to degradation of a phage genome in bacteria expressing the defense system. In some embodiments, a functional defense system comprises a restriction modification system. In some embodiments, a functional defense system comprises a gene editing system.

In some embodiments, a functional defense system does not lead to an abortive infection in bacteria expressing the defense system. In some embodiments, a functional defense system does not affect phage adsorption to bacteria expressing the defense system. In some embodiments, a functional defense system does not prevent phage genomic replication in bacteria expressing the defense system. In some embodiments, a functional defense system does not prevent phage lysogeny in bacteria expressing the defense system. In some embodiments, a functional defense system does not prevent circularization of a phage genome in bacteria expressing the defense system. In some embodiments, a functional defense system does not lead to degradation of a phage genome in bacteria expressing the defense system. In some embodiments, a functional defense system does not comprise a restriction modification system.

A skilled artisan would recognize that the term “anti-plasmid transformation” or “reducing the transformation of a host cell by a plasmid vector” encompasses an activity whereby a host cell, for example but not limited to bacteria, expressing a functional defense system as disclosed herein, has reduced transformation efficiency of an episomal plasmid, in comparison to the host cell of the same species under the same developmental stage (e.g. culture state) which does not express the functional defense system. Anti-plasmid transformation or reduction of transformation efficiency may be determined by, for example but not limited to, measuring plasmid transformation efficiency in the presence or absence of a defense system disclosed herein.

In some embodiments, a plasmid may undergo integration into the bacterial genome or may be episomal. In some embodiments, the plasmid is episomal. In some embodiments, the plasmid is integrative. In some embodiments, a Defense System disclosed herein, provides plasmid resistance.

The skilled artisan would appreciate that “plasmid resistance” or “anti-plasmid activity” encompasses an increase resistance of at least two-fold increased bacterial resistance towards plasmid transformation, in comparison to bacteria of the same species under the same developmental stage (culture state) which does not express the Defense System. In some embodiments, plasmid resistance may be manifested as viability of the bacteria. In some embodiments, plasmid resistance comprises decreased efficiency of transformation of the plasmid. According to a specific embodiment, the plasmid resistance is increased by at least two-fold, five-fold, ten-fold, twenty-fold, fifty-fold, one-hundred-fold, or one-thousand-fold.

Assays for testing plasmid resistance are well known in the art and include, but not limited to, a transformation assay such as described in Itaya and Tsuge [Methods Enzymol (2011) 498:427-47].

As used herein, the terms “the defense system” may in some embodiments be used interchangeably with the term “defense system” or “defense system” or “system”, having all the same meanings and qualities.

In some embodiments, different defense systems may be combined. Combination of defense systems, may in certain embodiments, provide multiple types of defense against foreign nucleic acid. For example, but not limited to defense against phage infection of phage from different families, or defense against phage infection and protection against conjugative elements, or defense against phage infection and resistance to plasmid transformation.

In some embodiments, a nucleic acid construct encodes an at least one defense system, said nucleic acid construct comprising nucleic acid sequences encoding polypeptide components of a defense system. In some embodiments, a nucleic acid construct described herein, encodes at least one defense system described herein. In some embodiments, a nucleic acid construct described herein, encodes at least one functional defense system described herein. In some embodiments, a nucleic acid construct comprises a nucleic acid encoding at least two different polypeptide components of the defense system. In some embodiments, a nucleic acid construct comprises a nucleic acid encoding at least one polypeptide components of the defense system. In some embodiments, a nucleic acid construct comprises a nucleic acid encoding at least three different polypeptide components of the defense system. In some embodiments, a nucleic acid construct comprises a nucleic acid encoding at least four two different polypeptide components of the defense system. In some embodiments, a nucleic acid construct comprises a nucleic acid encoding at least five different polypeptide components of the defense system.

In some embodiments, a nucleic acid construct encodes one component of the defense system, whereby multiple constructs may be used to assemble the functional defense system. In some embodiments, a nucleic acid construct encodes more than one component of the defense system but less than all of the components required for a functionally active defense system, whereby multiple constructs may be used to assemble the functional defense system. In some embodiments, a nucleic acid construct encodes more than one copy of a single component of the defense system, whereby multiple constructs may be used to assemble the functional defense system.

The skilled artisan would recognize that the “components” making up a functional defense system may in some embodiments, be also be referred to herein as the members of the “cassette of genes” identified to represent a defense system, wherein each component is encoded by a different gene. In some embodiments, a construct disclosed herein may comprise less than the full complement of polypeptides required for a functional defense system, whereby multiple constructs may be combined for expression of the functional defense system. In some embodiments, components comprise polypeptide molecules of a defense system. In some embodiments, components comprise non-coding RNAs. In some embodiments, components comprise promoters and other control elements.

In some embodiments, a defense system described herein comprises all of the polypeptide components as described herein. In some embodiments, a defense system comprises one of the polypeptide component as described herein. In some embodiments, a defense system comprises all of the polypeptide component as described herein. In some embodiments, a defense system comprises two of the polypeptide component as described herein. In some embodiments, a defense system comprises three of the polypeptide component as described herein. In some embodiments, a defense system comprises four of the polypeptide component as described herein. In some embodiments, a defense system comprises five of the polypeptide component as described herein. In some embodiments, a defense system comprises more than one copy of a particular polypeptide component of the defense system, as described herein.

A skilled artisan would appreciate that the term “nucleic acid sequence” and “polynucleotide”, which are interchangeably used herein, may encompass a single or double stranded nucleic acid sequence which is isolated and provided in the form of an RNA sequence, a complementary polynucleotide sequence (cDNA), a genomic polynucleotide sequence, and/or a composite polynucleotide sequences (e.g., a combination of the above).

In some embodiments, a nucleic acid construct disclosed herein encodes no more than 20, no more than 15, no more the 10 genes expression products. In some embodiments, a gene expression product comprises a component of a Defense System. In some embodiments, a gene expression product comprises a functional component of a Defense System. In some embodiments, a gene expression product comprises a portion of a component that is functional in a Defense System disclosed herein. In some embodiments, a gene expression product or a portion thereof is isolated. In some embodiments, a gene expression product or a portion thereof is comprised in a Defense System disclosed herein.

A skilled artisan would appreciate that the term “isolated” encompasses an element being at least partially separated from the natural environment, physiological environment e.g., a microorganism e.g., bacteria, e.g., a polypeptide, eg., a nucleic acid sequence.

A skilled artisan would appreciate that the term “polypeptide” encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein.

The polypeptides disclosed herein may be synthesized by any techniques known to those skilled in the art of peptide synthesis, for example but not limited to recombinant DNA techniques or solid phase peptide synthesis.

In some embodiments, a defense system comprises multiple nucleic acid constructs comprising nucleic acid sequences encoding polypeptide components of two or more functional the defense system. In some embodiments, the two or more functional the defense system do not naturally occur together in a cell in nature.

In some embodiments, each nucleic acid construct encoding a defense system or components of a defense system, further comprises a regulatory element or elements operably linked to the construct. In some embodiments, regulatory elements comprise a cis-acting regulatory element for directing expression of said nucleic acid sequence. In some embodiments, regulatory elements comprise a transmissible element for directing transfer of said nucleic acid sequence from one cell to another. In some embodiments, regulatory elements comprise a recombination element for integrating said nucleic acid sequence into a genome of a cell transfected with said construct. In some embodiments, regulatory elements comprise an element providing episomal maintenance of said construct within a cell transfected with said construct. In some embodiments, regulatory elements comprise a combination of a cis-acting regulatory element for directing expression of said nucleic acid sequence, a regulatory element comprising a transmissible element for directing transfer of said nucleic acid sequence from one cell to another, a regulatory element comprising a recombination element for integrating said nucleic acid sequence into a genome of a cell transfected with said construct, a regulatory element comprising an element providing episomal maintenance of said construct within a cell transfected with said construct, in any combination.

Defense Systems

In some embodiments, the defense systems disclosed herein may be a combination of defense systems. In some embodiments, a combination of the defense systems comprises an anti-phage system and an anti-plasmid system. In some embodiments, a combination of the defense systems comprises an anti-phage system and an anti-conjugative element system. In some embodiments, a combination of the defense systems comprises an anti-plasmid system and an anti-conjugative element system. In some embodiments, a combination of the defense systems comprises an anti-phage system, an anti-plasmid system, and an anti-conjugative element system. In some embodiments, a combination of the defense systems comprises an anti-phage system, an anti-plasmid system, or an anti-conjugative element system, or any combination thereof.

In some embodiments, introducing any of the defense systems disclosed herein, into eukaryotic cells, for example but not limited to bacteria cells, where they are not naturally found (therefore the defense system is non-naturally occurring), provides the host cells protection against phage infection.

In some embodiments, nucleic acid sequences or nucleic acid constructs may further include nuclear targeting signals (NLS) so that a nucleic acid sequence encoding a non-naturally polypeptide component, may be directed to the nucleus for expression. In some embodiments, nucleic acid sequences or nucleic acid constructs may further include nuclear targeting signals (NLS) so that multiple nucleic acid sequences encoding non-naturally polypeptide components or a defense system described herein, may be directed to the nucleus for expression. In some embodiments, nucleic acid sequences or nucleic acid constructs may further include nuclear targeting signals (NLS) so that combinations of nucleic acids encoding defense systems (polypeptides) described herein, may be directed to the nucleus for expression.

In some embodiments, introducing any of the defense systems disclosed herein, into eukaryotic cells, for example but not limited to bacteria cells, where they are not naturally found (therefore they are non-naturally occurring), provides the host cells protection against introduction of external DNA. In some embodiments, introducing any of the defense systems disclosed herein, into eukaryotic cells, for example but not limited to bacteria cells, where they are not naturally found, provides the host cells protection against introduction of external plasmid DNA. In some embodiments, introducing any of the defense systems disclosed herein, into eukaryotic cells, for example but not limited to bacteria cells, where they are not naturally found, provides the host cells protection against introduction of external conjugative elements.

In some embodiments, a combination of the defense systems comprises combining an anti-phage system and another anti-phage system. In some embodiments, a combination of the defense systems comprises combining an anti-phage system and more than one other anti-phage system. In some embodiments, a combination of the defense systems comprises combining an anti-phage system and two other anti-phage systems. In some embodiments, a combination of the defense systems comprises combining an anti-phage system and three other anti-phage systems. In some embodiments, a combination of the defense systems comprises combining an anti-phage system and four other anti-phage systems. In some embodiments, a combination of the defense systems comprises combining an anti-phage system and five other anti-phage systems. In some embodiments, a combination of the defense systems comprises combining an anti-phage system and six other anti-phage systems. In some embodiments, a combination of the defense systems comprises combining an anti-phage system and seven other anti-phage systems. In some embodiments, a combination of the defense systems comprises combining an anti-phage system and eight other anti-phage systems. In some embodiments, a combination of the defense systems comprises combining an anti-phage system and nine other anti-phage systems. In some embodiments, a combination of the defense systems comprises combining an anti-phage system and ten other anti-phage systems. In some embodiments, a combination of the defense systems comprises combining an anti-phage system and more than 10 other anti-phage systems. In some embodiments, a combination of the defense systems comprises combining an anti-phage system with between 2-15 other anti-phage systems. In some embodiments, a combination of the defense systems comprises combining an anti-phage system with between 2-7 other anti-phage systems. In some embodiments, a combination of the defense systems comprises combining an anti-phage system with between 7-15 other anti-phage systems.

In some embodiments, an at least one defense system comprises any of the defense systems described herein. In some embodiments, an at least one defense system comprises any combination of the defense systems described herein. In some embodiments, wherein a defense system comprises at least two different polypeptide components (See below and Table 18), an at least one defense system comprises at least two different polypeptide components of the defense system.

Below are non-limiting examples of defense systems, which in some embodiments may be used independently, and in other embodiments may be used in combinations.

The ZORYA Defense System

In some embodiments, a defense system disclosed herein comprises a ZORYA anti-phage defense system. In some embodiments, a ZORYA defense system (Defense System Ia or Defense system Ib) provides a host cell with resistance to entry of foreign nucleic acid invasion. In some embodiments, a host cell expressing a functional ZORYA defense system (Defense System Ia; or Defense System Ib) provides the host cell resistance foreign nucleic acid invasion.

In some embodiments, a ZORYA defense system provides a host cell with resistance to at least one phage. In some embodiments, a ZORYA Type I defense system provides a host cell with resistance to at least one phage. In some embodiments, a ZORYA Type II defense system provides a host cell with resistance to at least one phage. (Table 8)

In some embodiments, expression of a ZORYA defense system in bacteria protects the bacteria from phage infection. In some embodiments, expression of a ZORYA Type I defense system in bacteria protects the bacteria from phage infection. In some embodiments, expression of a ZORYA Type II defense system in bacteria protects the bacteria from phage infection.

In some embodiments, a ZORYA defense system (Defense System Ia or Defense system Ib) provides a host cell with resistance to plasmid transformation. In some embodiments, a host cell expressing a functional ZORYA defense system (Defense System Ia or Defense System Ib) provides a host cells with resistance to plasmid transformation.

In some embodiments, a ZORYA defense system (Defense system Ia or Ib) provides a host cell with resistance to entry of conjugative elements. In some embodiments, a host cell expressing a function ZORYA defense system (Defense System Ia or Ib) provides the host cell resistance from entry of conjugative elements.

As used herein, the term “a ZORYA anti-phage defense system” may be used interchangeably with the term “a Defense System I”, having all the same meanings and qualities. Further, in some embodiments, the term “a ZORYA Type I defense system” may be used interchangeably with the term “a Defense System Ia”, having all the same meanings and qualities. In some embodiments, the term “a ZORYA anti-phage defense system” may be used interchangeably with the term “a Defense System Ia”, having all the same meanings and qualities. Further, in some embodiments, the term “a ZORYA Type I defense system” may be used interchangeably with the term “a Defense System Ia”, having all the same meanings and qualities. In some embodiments, the term “a ZORYA anti-phage defense system” may be used interchangeably with the term “a Defense System Ib”, having all the same meanings and qualities. Further, in some embodiments, the term “a ZORYA Type I defense system” may be used interchangeably with the term “a Defense System Ib”, having all the same meanings and qualities.

In some embodiments, a ZORYA defense system provides a host cell with resistance to plasmid transformation. In some embodiments, a ZORYA Type I defense system provides a host cell with resistance to plasmid transformation. In some embodiments, a ZORYA Type II defense system provides a host cell with resistance to plasmid transformation.

As used herein, the term “ZORYA system” may be used interchangeably in some embodiments with “ZORYA defense system”, “ZORYA the defense system”, “ZORYA defense system”, “ZORYA anti-phage system”, “ZORYA Type I defense system”, “ZORYA Type II defense system”, “Defense System Ia” and “Defense System Ib”.

In some embodiments, a microbial species does not comprise an endogenous Defense System I. In some embodiments, a microbial species does not comprise an endogenous Defense System Ia. In some embodiments, a microbial species does not comprise an endogenous Defense System Ib. In some embodiments, a microbial species does not express an endogenous Defense System I. In some embodiments, a microbial species does not express an endogenous Defense System Ia. In some embodiments, a microbial species does not express an endogenous Defense System Ib. In some embodiments, a microbial species does not express an endogenous functional Defense System I. In some embodiments, a microbial species does not express an endogenous functional Defense System Ia. In some embodiments, a microbial species does not express an endogenous functional Defense System Ib.

In some embodiments, a bacterial species does not comprise an endogenous Defense System I. In some embodiments, a bacterial species does not comprise an endogenous Defense System Ia. In some embodiments, a bacterial species does not comprise an endogenous Defense System Ib. In some embodiments, a bacterial species does not express an endogenous Defense System I. In some embodiments, a bacterial species does not express an endogenous Defense System Ia. In some embodiments, a bacterial species does not express an endogenous Defense System Ib. In some embodiments, a bacterial species does not express an endogenous functional Defense System I. In some embodiments, a bacterial species does not express an endogenous functional Defense System Ia. In some embodiments, a bacterial species does not express an endogenous functional Defense System Ib.

A ZORYA Type I defense system (Defense System Ia) comprises, in some embodiments, a nucleic acid construct comprising a nucleic acid sequence encoding a ZorA polypeptide comprising a pfam01618 domain, a ZorB polypeptide comprising a pfam13677 domain or a pfam00691 domain or a combination thereof, a ZorC polypeptide comprising a pfam15611 domain, and a ZorD polypeptide comprising a pfam00176 domain or a pfam00271 domain or a combination thereof. In some embodiments, a ZORYA Type I defense system comprises a nucleic acid construct comprising a nucleic acid sequence encoding a ZorA polypeptide, a ZorB polypeptide, a ZorC polypeptide, and a ZorD polypeptide. In some embodiments, a ZORYA Type I defense system comprises a nucleic acid construct comprising a nucleic acid sequence comprising a zorA gene, a zorB gene, a zorC gene, and a zorD gene.

In some embodiments, a ZORYA Type I defense system (Defense System Ia) comprises a ZorA polypeptide comprising a pfam01618 domain, a ZorB polypeptide comprising a pfam13677 domain or a pfam00691 domain or a combination thereof, a ZorC polypeptide comprising a pfam15611 domain, and a ZorD polypeptide comprising a pfam00176 domain or a pfam00271 domain or a combination thereof. In some embodiments, a ZORYA Type I defense system (Defense System Ia) having an anti-phage activity comprises a ZorA polypeptide comprising a pfam01618 domain, a ZorB polypeptide comprising a pfam13677 domain or a pfam00691 domain or a combination thereof, a ZorC polypeptide comprising a pfam15611 domain, and a ZorD polypeptide comprising a pfam00176 domain or a pfam00271 domain or a combination thereof.

In some embodiments, a ZORYA Type I defense system (Defense System Ia) comprises at least two polypeptide components selected from a ZorA polypeptide comprising a pfam01618 domain, a ZorB polypeptide comprising a pfam13677 domain or a pfam00691 domain or a combination thereof, a ZorC polypeptide comprising a pfam15611 domain, and a ZorD polypeptide comprising a pfam00176 domain or a pfam00271 domain or a combination thereof. In some embodiments, a ZORYA Type I defense system (Defense System Ia) having an anti-phage activity comprises at least two polypeptide components selected from a ZorA polypeptide comprising a pfam01618 domain, a ZorB polypeptide comprising a pfam13677 domain or a pfam00691 domain or a combination thereof, a ZorC polypeptide comprising a pfam15611 domain, and a ZorD polypeptide comprising a pfam00176 domain or a pfam00271 domain or a combination thereof. In some embodiments, a ZORYA Type I defense system (Defense System Ia) comprises at least three polypeptide components selected from a ZorA polypeptide comprising a pfam01618 domain, a ZorB polypeptide comprising a pfam13677 domain or a pfam00691 domain or a combination thereof, a ZorC polypeptide comprising a pfam15611 domain, and a ZorD polypeptide comprising a pfam00176 domain or a pfam00271 domain or a combination thereof. In some embodiments, a ZORYA Type I defense system (Defense System Ia) having an anti-phage activity comprises at least three polypeptide components selected from a ZorA polypeptide comprising a pfam01618 domain, a ZorB polypeptide comprising a pfam13677 domain or a pfam00691 domain or a combination thereof, a ZorC polypeptide comprising a pfam15611 domain, and a ZorD polypeptide comprising a pfam00176 domain or a pfam00271 domain or a combination thereof. In some embodiments, a ZORYA Type I defense system (Defense System Ia) comprises at least four polypeptide components selected from a ZorA polypeptide comprising a pfam01618 domain, a ZorB polypeptide comprising a pfam13677 domain or a pfam00691 domain or a combination thereof, a ZorC polypeptide comprising a pfam15611 domain, and a ZorD polypeptide comprising a pfam00176 domain or a pfam00271 domain or a combination thereof. In some embodiments, a ZORYA Type I defense system (Defense System Ia) having an anti-phage activity comprises at least four polypeptide components selected from a ZorA polypeptide comprising a pfam01618 domain, a ZorB polypeptide comprising a pfam13677 domain or a pfam00691 domain or a combination thereof, a ZorC polypeptide comprising a pfam15611 domain, and a ZorD polypeptide comprising a pfam00176 domain or a pfam00271 domain or a combination thereof.

In some embodiments, a Defense System Ia comprises a ZorA polypeptide comprising a pfam01618 domain or comprising an amino acid sequence having at least 80% identity to a sequence selected from the group referenced in Table 8 rows 2-1830 columns J and K; or a ZorB polypeptide comprising a pfam13677 domain or a pfam00691 domain or a combination thereof, or comprising an amino acid sequence having at least 80% identity to a sequence selected from the group referenced in Table 8 rows 2-1830 columns N and O; or a ZorC polypeptide comprising a pfam15611 domain or comprising an amino acid sequence having at least 80% identity to a sequence selected from the group referenced in Table 8 rows 2-1174 columns R and S; or a ZorD polypeptide comprising a pfam00176 domain or a pfam00271 domain or a combination thereof, or comprising an amino acid sequence having at least 80% identity to a sequence selected from the group referenced in Table 8 rows 2-1174 columns V and W; or said Defense System Ia comprises any combination of polypeptide components ZorA, ZorB, ZorC, and ZorD.

A skilled artisan would appreciate that the term “comprising” encompasses inclusion of the recited elements, but not excluding others which may be optional. For example, but not limited to a defense system, a defense system may comprise a single polypeptide component, or may comprise multiple polypeptide components. For example, but not limited to a Defense System Ia, in some embodiments a Defense System Ia comprises at least two different polypeptide components selected from ZorA, ZorB, ZorC, and ZorD. In some embodiments, a Defense System Ia comprises at least three different polypeptide components selected from ZorA, ZorB, ZorC, and ZorD. In some embodiments, a Defense System Ia comprises polypeptide components ZorA, ZorB, ZorC, and ZorD.

A skilled artisan would appreciate that the phrase “consisting essentially of” may encompass, for example but not limited to, a defense system that includes the recited elements but exclude other elements that may have an essential significant effect on the performance of the method. In some embodiments, a Defense System Ia consists essentially of polypeptide components ZorA, ZorB, ZorC, and ZorD.

A skilled artisan would appreciate that the term “consisting of” may encompass, for example but not limited to, a defense system that includes the recited elements but not more than traces of other elements. Thus, in some embodiments, a Defense System Ia consists of a ZorA polypeptide, a ZorB polypeptide, a ZorC polypeptide, and a ZorD polypeptide.

Embodiments defined by each of these transition terms are within the scope of this invention.

A ZORYA Type II defense system (Defense System Ib) comprises, in some embodiments, a nucleic acid construct comprising a nucleic acid sequence encoding a ZorA polypeptide comprising a pfam01618 domain, a ZorB polypeptide comprising a pfam13677 domain or a pfam00691 domain or a combination thereof, and a ZorE polypeptide comprising a pfam01844 domain. In some embodiments, a ZORYA Type II defense system (Defense System Ib) comprises a nucleic acid construct comprising a nucleic acid sequence encoding a ZorA polypeptide, a ZorB polypeptide, and a ZorE polypeptide. In some embodiments, a ZORYA Type II defense system (Defense System Ib) comprises a nucleic acid construct comprising a nucleic acid sequence comprising a zorA gene, a zorB gene, and a zorE gene.

In some embodiments, a ZORYA Type II defense system (Defense System Ib) comprises a ZorA polypeptide comprising a pfam01618 domain, a ZorB polypeptide comprising a pfam13677 domain or a pfam00691 domain or a combination thereof, and a ZorE polypeptide comprising a pfam01844 domain. In some embodiments, a ZORYA Type II defense system (Defense System Ib) having an anti-phage activity comprises a ZorA polypeptide comprising a pfam01618 domain, a ZorB polypeptide comprising a pfam13677 domain or a pfam00691 domain or a combination thereof, and a ZorE polypeptide comprising a pfam01844 domain.

In some embodiments, a Zorya Type II defense system (Defense System Ib) comprises a ZorE polypeptide and ZorA polypeptide. In some embodiments, a Zorya Type II defense system (Defense System Ib) comprises a ZorE polypeptide and ZorB polypeptide. In some embodiments, a Zorya Type II defense system (Defense System Ib) comprises a ZorE polypeptide, ZorA polypeptide, and a ZorB polypeptide. In some embodiments, a Zorya Type II defense system (Defense System Ib) comprises a ZorE polypeptide comprising a pfam01844 domain or a COG3183 domain or a combination thereof. In some embodiments, a Zorya Type II defense system (Defense System Ib) having an anti-phage activity comprises a ZorE polypeptide comprising a pfam01844 domain or a COG3183 domain or a combination thereof.

In some embodiments, a Defense System Ib comprises a ZorA polypeptide comprising a pfam01618 domain or comprises an amino acid sequence having at least 80% identity to a sequence selected from the group referenced in Table 8 rows 2-1830 columns J and K; or a ZorB polypeptide comprises a pfam13677 domain or a pfam00691 domain or a combination thereof, or comprises an amino acid sequence having at least 80% identity to a sequence selected from the group referenced in Table 8 rows 2-1830 columns N and O; or a ZorE polypeptide comprising a pfam01844 domain or a COG3183 domain or a combination thereof or comprises an amino acid sequence having at least 80% identity to a sequence selected from the group referenced in Table 8 rows 1175-1830 columns Z and AA; or any combination thereof.

In some embodiments, a ZORYA Type I defense system having an anti-phage activity comprises a nucleic acid construct comprising a nucleic acid sequence encoding a ZorA polypeptide, a ZorB polypeptide, a ZorC polypeptide, and a ZorD polypeptide. In some embodiments, a ZORYA Type I defense system having an anti-phage activity comprises a nucleic acid construct comprising a nucleic acid sequence comprising a zorA gene, a zorB gene, a zorC gene, and a zorD gene. In some embodiments, a ZORYA Type II defense system having an anti-phage activity comprises a nucleic acid construct comprising a nucleic acid sequence encoding a ZorA polypeptide, a ZorB polypeptide, and a ZorE polypeptide. In some embodiments, a ZORYA defense system having an anti-phage activity comprises a nucleic acid construct comprising a nucleic acid sequence comprising a zorA gene, a zorB gene, and a zorE gene.

In some embodiments, the ZORYA anti-phage defense system comprises a nucleic acid construct comprising nucleic acid sequences encoding a cassette comprising zorA, zorB, zorC, and zorD genes. In some embodiments, a construct comprising the ZORYA defense system encodes one component of the defense system, whereby multiple constructs may be used to assemble the functional defense system. In some embodiments, the components of a ZORYA Type I defense system comprise genes zorA, zorB, zorC, and zorD. In some embodiments, the components of a ZORYA Type I defense system consist of genes zorA, zorB, zorC, and zorD. In some embodiments, the components of a ZORYA Type I defense system comprise nucleic acid sequences encoding a ZorA polypeptide, a ZorB polypeptide, a ZorC polypeptide, and a ZorD polypeptide. In some embodiments, the components of a ZORYA Type I defense system consist of nucleic acid sequences encoding a ZorA polypeptide, a ZorB polypeptide, a ZorC polypeptide, and a ZorD polypeptide.

In some embodiments, a Zorya Type I (Defense System Ia) gene cassette comprises the nucleic acid sequence:

(SEQ ID NO: 14; Construct 49 Table 4)

| | |
|---|---|
| 1 | tgtaaatcct cggggtagat cgcagcgaaa gagcaaccat gccttatcgc aaattcagtt |
| 61 | ttggaatgca gttcgcaaaa tgatacccgg tacacttacg attacaaatg cgcggtataa |
| 121 | agaagtttgc atttgatcgg tgaataatag gaaactcagt cactgtttcg tctgcaaaat |
| 181 | cagccatttc gccttcatac gtagttcttc ggttgaatag taaggtctat catgctgcat |
| 241 | cgaacggtga ttttgatgga tgttggaagt gagtagctgt acgtaatttt tgattaaaag |
| 301 | taatcaaaat gttgccaatg gttatgctat cgtgtgtttt ttgaatatat aacaggtaaa |
| 361 | actatgtcct ggcttaattc tgtcttattg gcgcttacct cggtgcaacc ttatatggta |
| 421 | ccggcaaccg ttatcggcct cgtcagtttt gcattcctct gttttatttt cttttatttt |
| 481 | tttagagcag ttaaaattat taacggcctg aaaaaatata ctcagtcaat taatggcatc |
| 541 | gaaaataatg agcctggtaa tcagcttcag catctgcaaa gtttgtttgt acagcctgag |
| 601 | ttaaagcacg cctggaatga attcgaagaa tcgttgcact cacaatatga actggaggac |
| 661 | ggggaagaga aaatcgtccg tattagggct actgcaccga gcgccagttt cttctcggag |
| 721 | caacaactgg tcgatattcc gctgaacacc gaatttttca aacacctgcc cggtattctg |
| 781 | accggtgtgg ggattatcgg taccttctac ggtctgatga ttggtctcaa ccacttcgac |
| 841 | cccagcacac cagagcaggt ctccagcagc gtcaataatc tgcttcgcga tgtgctgtac |
| 901 | gccttcctcg gttcagcatt tgcgattacg ttctctatcc ttatcacctg gctggagaaa |
| 961 | ttctgtctgg cgaaatgcta caaatatctg gaaaaattca ccgcagctct cgacgccctc |
| 1021 | tatgacagcg gtgttggcga ggagtacctt gcctcactgg taaaatccag taatgaaagc |
| 1081 | gcgacccagg cacgccatct gaaagagagc ctggttaccg atttacgcga tatgctgctg |
| 1141 | catctggcca acagccagaa agtggaaaac gagcggctag cgaccaccct cagcaccacc |
| 1201 | tatcgcgaaa caggacaaca gtttgccgag caagtcagcg gtgcgattga aaacagcctg |
| 1261 | aaatcaccgc tggataaaat agcgggcgcg gttcagaccg ccagtggcga tcaaagcggc |
| 1321 | atggtacaaa acatgctgca ggatgtgctg accgcgttta tggcgaaact cgacaccacc |
| 1381 | tttggtcagc agtttactaa tctcaacgaa atgatgggac aaaccgttgg cgctattcag |
| 1441 | acaatgcaaa ccggtttcgg cgcgttatta caggacatgc gtcaggtgag cgatgattcc |
| 1501 | cgccagggca gcgcgcagct catcgagcaa ctgttatcag agatgaaatc tggccagcaa |
| 1561 | gctatgcagg ctggcatgaa tgacatgctc accagccttc agacctcggt ggctaaaatt |
| 1621 | ggcgcagaag gtgaaggtgc cggcgagcga atggctcgcc aactagagaa aatgttcgct |
| 1681 | gatagcgagg cgcgagaaaa agcccaggca gaacatatga ccgcctttat tgaagccatt |
| 1741 | cagaattcag tgcagcaggg acaaagcgcc acaatggaaa aaatggcagc ctctgtcgag |
| 1801 | tcgcttggtg aacaactggg tagcctgttt gggcagattg ataagggtca gcaacagatt |
| 1861 | tcagcaaatc agcaagcgaa tcagcagtcc ctgcacgaac agactcagcg ggtaatgagt |
| 1921 | gaggtagacg atcagattaa acaattggtc gaaactgttg ccagccagca tcagggaaca |
| 1981 | actgaaacac ttcgtttact cgcagaacaa actaaccgcc agattcagga tatgcatacc |
| 2041 | ggcgcggaca aaatgcgtct ggcggccgag cgctttgaac atgccggtga tcgggtgtcg |
| 2101 | gaagcaaacc accttaccgc tgacgtgctg aataaagcgc aatcggcagg ttcatcactc |
| 2161 | tcccttgcca ccagcgaact cacctctgtg gtggctgatt atcgtaacaa tcgggaagcg |
| 2221 | gttagcaaat ccattgccat gctggagttg ctcgccgcaa atacgcagtc tgaacaaacc |
| 2281 | acgcgcaccc aattcatcgc cgaccttaag cagcacggtg agcgtctgca gagctataac |
| 2341 | cgggaagcac aggcctttat ggaaaatgtc agtgacgtgc tagggaaagg gtttgaagac |
| 2401 | ttctctgaag gtgtttcacg cagcctggat aaaacgctgg gcaaactgga cgttgaaatg |
| 2461 | gcgaaggctt ctactctgct cgcgggttct gtcgaacaga tcggagaaag cgtcagcgag |
| 2521 | cttgatgatg tcctgtcacg cgttcgtgcc taaggggtga cggctgatgt ttggaaatgc |
| 2581 | atttggcgtt aaaaaacgcc gcagcgatga agcagagaaa cccttctgga tctcctatgc |
| 2641 | cgatttgatg actgccatga tggtgttatt tctggtcgtt atggttgcgt cgctgagttc |
| 2701 | cgttacgcag cgtattcaac gtgcggagca aggtgaaaaa acacgggggc aggatatttc |
| 2761 | cagattgtgt gagcgcctgg aattgcatgc ccgcaacgtg aacaaaacca ttgtggtgga |
| 2821 | ttgccatgat aaccgtatca gttttggtga agccgggcgt ttcgatcaca accaattttt |
| 2881 | cctgaacgct gaggggcaaa aagccttgca ggatgtggtt cctcttgtgc tggaagcgtc |
| 2941 | caacagtgaa gaaggtaaaa aatggtttaa gcagatcgtt attgaagggt ttaccgacac |
| 3001 | cgacggttcg tacttataca acctacatct ctccctgcaa cgttctgagt gggtaatgtg |
| 3061 | tagtctgctc gatagccgca gccctctgca aaaaaatatc tccgcagaac agcagctcca |
| 3121 | aatccgtaaa cttttcctcg cgggtggtgt ctccttcaat aatgccaagg aaagcaagga |
| 3181 | agccagccgc cgcgtcgagt tgcgcatgca gttcttcggg cttaaagata aacgtgataa |
| 3241 | ggccgatgag gtggatttcc cccccgtcgt caataaagaa gtttgccagt tggtaatgcc |
| 3301 | cctatgacgc ctgcactgaa ttctctttct caacgcattg cagcgaggct ctcttccagc |
| 3361 | caacgggatg accattatct gcataatgat ttccacgcgc tggcctcagc tgctctcgac |
| 3421 | atggaaaagc ggtttgataa ggcagaaaaa atccctttac cgccacaaaa aatgcgactg |
| 3481 | gcagctctgc gtcgccttcg ccttgcacaa gaactcacgg agcgagagtg gcggatggtg |
| 3541 | ttttatggtc tcgccgataa tgatccgtca tatcctgacc agccagtact gctggaggat |
| 3601 | gatacgtttt tccctgaagt aaacaatgca atcaaaaagc ggcttgaaac taaaaccctg |
| 3661 | aaacgccgag actgggccgc actctgttca agctattttg cttaccagaa cccatcacct |
| 3721 | gagacaaacc cgcactggtg cgtactgcgt ggtcatatcg ctcagggtta catggtggtg |
| 3781 | aaagcggcta tacgacgaga aaaatcgtgg atgaaaacca ttgagtttta tcacgatatt |
| 3841 | tttaccccgc aggcgggagg cgtgatctcc agacagttac ttgcgggtga gagcaattca |
| 3901 | ctctcgtcat tagaaaaaat cgcgcaaatc cctgatagca gttggttgtg gaaacgtatt |
| 3961 | ttcacggtgc ttttagcgca actggacaca ctcgacgatc cccagtttct ggacaaaatt |
| 4021 | agctggttgt taggtctggc tgcccagtgg gtacgtttcc gtgatgacat catgactgcg |
| 4081 | accttgactc gctattacca ttctatttat cgcgatcagg ctcattcagc tttgaagcag |
| 4141 | gccgcgctgg aatactggga taacccgcaa ctgaaaagcc agcagaataa atggcatcag |
| 4201 | tatgtctctg agccggtggc tgcgatggtc cggggctggc tggcaaaaca agatctgatg |
| 4261 | catttctttg aattgctgcg cggtaatggc gatgttgacc aggcacgatt gcactattgg |
| 4321 | ctacgtttcg ccaatcagat gggatttacc cgtatcgtca tggggactga tgcctggcaa |
| 4381 | gaccgtggta gtgactttgt gaaattccgc gaggagaata agggacgatt gagttactta |
| 4441 | cgaggtggac gtaactttga taacgccatg attatgcaga tcaacgatta tctgttcgtt |
| 4501 | gagttttccg ggacagggaa tgcaatgtat gcctatcgaa taggtcacgc gccatttaac |
| 4561 | cctgaatcac gcacgcttga tatcaacatt catctaaaag ataaggggcg ctgcgtgctc |
| 4621 | cgactgcccc atacaccgcg cgcagaaggg tataacaaag tccggataac cggctggatg |
| 4681 | ttgaaatacg acgacgagct gcgtcagttg ggcattcggt ggatggcaga agaagccatc |
| 4741 | aagtttgtcg ataagaaagc gtcctctccc gcctctatgt ccgacattaa aatcatcaac |
| 4801 | ccgttgcgtg atacggctat acagcatctg gttgaaggta gttcatgtat cgtcagcgat |
| 4861 | aatcgacaaa aaggtggcgt actgtccgta caactcaata ctcctgacga caccatagaa |
| 4921 | agagagttgt tacggctagg ctttgcgccc gtggctaaag aaccgcatcg ttactggatc |
| 4981 | aaataatgct gaaacgtctg ttaagtaaac tgacgggaaa ccgtcagcag atagaacatc |
| 5041 | acctaaaaaa tcagtaccag gttgaggaaa acggacttag ctttccgcta tcgctggttg |
| 5101 | atgattcgca gctttgggca ctggcatcct ggctagaaca actggctgaa gaagactatc |
| 5161 | tgatatcact tacagaccgg tggttgttga gttgggacgc actgtatcgc ctgctggagg |
| 5221 | atgaggaaca tgccagcagc ctgcctctta tcggtgtacc tgacgtactg cctttacgcg |
| 5281 | caagcctgag ttcacgagga gcattaagcg atagcgattt tcgcgtctgg attgctgaat |
| 5341 | gggctacgct tccagcccgc aagccgatcc gtttcagtcg taccggtgcc attttaaccc |
| 5401 | atgaaaatca gcaatacctg ttatcgcgtg aaaactgggc actcttacag gcaacagaac |
| 5461 | aactcagtgc acagaaaaat cagactcctg gtgaaaccac taaccagttg ggatgggccg |
| 5521 | ctatccgcaa atgtgcgaag caagcagccg caaaatttga tgattattta gaaaaaacgc |
| 5581 | atgtcgtcaa accaacttca ttgtcattac gtctgcgtaa agcaacggtt gccgatactg |
| 5641 | ctgttatcga aattgagccc catttcgagg accagcctgc taactggcta ggcagttttg |
| 5701 | acaaaaactc gcaggttcac gatagctacc gcatccctgg agagaatggc gaactcagcc |
| 5761 | atgtcattat ccctcccgaa gtgaaagaag tcctgaattc aatacactcg attccaagcc |
| 5821 | gtcgagtggc cggaagcgaa gcgctttcct ttgttcgcaa tccttatacc ttcctgggtg |
| 5881 | aagatgccgc cagtgttatt gcccctgaag aacatgaaca ggcactgttt gatgccagga |
| 5941 | ttttctttca tcatttcagg ttgataccgc aactgaatgc ggaaaataag atcgcagaag |
| 6001 | ttacgctggt acttgagcct gtttcacctg ttccccagcc agaaatcact tttgggttct |
| 6061 | cagccccccg ggagctggat aaattcatac agcaactcgg tattagcgtc gctgcacaga |
| 6121 | tgcctgccgg ttcatggcaa ggttacgagc tggaattaag tcagttcact gaacagcagt |
| 6181 | ggcacgattg ccaggcactt ctaactcgct ggcagcaaga aatagaagga aaagaattta |
| 6241 | gcgatgtgct ggatattgcg aagtatggcg atcgcgtgat tggcattggt gaatttgaaa |
| 6301 | aaatctcctc tccctggctg accaaagcgc agagtgaaaa ctggcttcca gatgacatcg |
| 6361 | atttctccgc gttttcagtc gaaacactgt ctggctggca acctgaaaac cttcaccatt |
| 6421 | tcgatgaact gcaggagaga attactcagg cagaagctgt aggtgaaacg catatcaccg |
| 6481 | caccatggaa tgacagccaa ttaccgttgg atgctgctaa aaccttcagt aagaactggg |
| 6541 | aaaaacagca aagcactgca aatgaatctc aaggcaacgt tgcagataaa acggcccgag |
| 6601 | ccgtactcaa aattgagcag aatattgaag aaacggccta catcaaacag cgccgcaatt |
| 6661 | cactcctcaa tgcgcgtcat gccgagcctg aaattcctct gagccttaaa gagcatatcc |
| 6721 | gactcaaaga ccatcaacgt gaaggcgtag cctggctcca gcaactcttc cttcgttcac |
| 6781 | ctgaggaaac cgcaggttgc ctgctggcgg atgatatggg gcttgggaaa acgctgcaaa |
| 6841 | ttctgagctt cctggtatgg ttcattgaaa aattcccgca agaaccgcca agtctcattg |
| 6901 | ttgctcctgt ctctctttta gataactggg aacgtgaact ggacaatttc ttctataccg |
| 6961 | caggaatacc ggtattgaaa ttgtatggcg agaccattaa agcggtgaaa tatcccaaac |
| 7021 | aggctatccc tgctcatctg caatctcagg ggatcaaaaa cctgcttaaa cctggctggc |
| 7081 | agggtgaggc gaaaatcatc ctgacaacct acgaaacgct tcgcgaccag gagttttctc |
| 7141 | tggcacgcca gccgtggtcc attatggtgt gcgacgaggc acaaaaaata aaaaacccag |
| 7201 | ctgcgttaat cacacatgcg gccaacgcgg tacaagccag gtttaaagtg gcatgtacag |
| 7261 | gaacacctgt cgaaaacaca cttgtcgacc tgtggagctt atttgatttt gcccagccag |
| 7321 | gattgttagg ggcactaaat gaatttggta aacactatgt tcgccccatc gagaacgaag |
| 7381 | atggccgcga tacggaacgg ttggaaagcc tgcgagcgct gatagaacct cagactttac |
| 7441 | gccgaaccaa agaagaggtt gcgcgtgatt tacctcagaa aattgaagtg gaaagctgca |
| 7501 | agcaattgac gttgtctggc gtgcaaaaac agctctacct ttcgtccgtt gcaaattggc |
| 7561 | aacaacagca agcactgagc gaagggatgc agcaggccgg aaccggtatg ttaggtttgt |
| 7621 | tacaccgact gaaactcatc tgtgcacacc ctgcagtagt taatccagaa ccacgttttc |
| 7681 | gtgataactc acccaagctt aactggctgc tgaaaatact cgcagaacta aaacacacca |
| 7741 | cgaaagataa ggtcattatt tttacggagc ttcgagattt acagcgcgaa cttcaacatg |
| 7801 | cgatccacca aaagtttggt ttccgccctg tcattatcaa tggtgatacc agcactaaaa |
| 7861 | gtcaaagtca aaacagccgc cagcgcctga ttgatgattt tcaggctcag cctgggtttg |
| 7921 | gggtgattat tctctctacg gttgccgtcg gattcggcgt taacgtccag aaagccaatc |
| 7981 | atgttattca tttcactcgc tgctggaacc cagccaaaga agatcaggca acagaccggg |
| 8041 | cgtaccgaat tgggcaaact aaagatgtgt atgtctatta cccgacggtg aaggacactg |
| 8101 | aaatcaccac atttgaagaa acactggatg accttctgca gcgtcgacgt gcattggcca |
| 8161 | gagatatgct ctgcgctact cccgatttga gtggtgcgga ttttgaggcc attttgaagg |
| 8221 | gggcttaacc gatacgagtg gcaataccac cagcagtcga accgattcat ttgaaacgac |
| 8281 | acgtagccgc gatggatctg tatcacctac cgacagcttg ggaaagtccc gaattacaaa |
| 8341 | tgttcagggg aataccataa cctgccggac ttattcgttt ggaacgacgt gttacgactt |
| 8401 | agtccgaggg aaacatgcca ggtaaaaacc gaatatccat cctcagcttt agcaaatacc |
| 8461 | tccccatact cgctacttca tattgagaag atattgtgtt gctcatattg ctttgagcat |
| 8521 | gctcttcagc gtttacatgt ggatcgtact accctgatga tccgatgcgt tgtttactgc |
| 8581 | ttttaacctc tatatacaaa acccccacta aatatttttc tgagactaac ggtaaatcat |
| 8641 | tatctatcat gtagttacga tagaattgca tgtatctggc acaaaataag gtttcattac |
| 8701 | gttctatctt gttaaatata aagggatcat ttcctcccaa agtcacaatc |

The coding regions for each of the zorA, zorB, zorC, and zorD gene sequences within this embodiment of a Zorya Type I cassette (SEQ ID NO: 14) are as follows: nucleotides 364-2553 encode an embodiment of a ZorA polypeptide; nucleotides 2567-3307 encode an embodiment of a ZorB polypeptide; nucleotides 3304-4986 encode an embodiment of a ZorC polypeptide; and nucleotides 4986-8228 encode an embodiment of a ZorD polypeptide.

In some embodiments, a Defense System Ia comprising a ZorA polypeptide, a ZorB polypeptide, a ZorC polypeptide, and a ZorD polypeptide, is encoded by the nucleic acid sequence set forth in SEQ ID NO: 14. In some embodiments, a Defense System Ia comprising a ZorA polypeptide, a ZorB polypeptide, a ZorC polypeptide, and a ZorD polypeptide, is encoded by a nucleic acid sequence having at least 80% homology to the sequence set forth in SEQ ID NO: 14. In some embodiments, a Defense System Ia comprising a ZorA polypeptide, a ZorB polypeptide, a ZorC polypeptide, and a ZorD polypeptide, is encoded by a nucleic acid sequence having at least 85% homology, or 90% homology, or 95% homology, or 97% homology, or 98% homology, or 99% homology to the sequence set forth in SEQ ID NO: 14. In some embodiments, a Defense System Ia comprising a ZorA polypeptide, a ZorB polypeptide, a ZorC polypeptide, and a ZorD polypeptide, is encoded by a nucleic acid sequence having at least 80% identity to the sequence set forth in SEQ ID NO: 14. In some embodiments, a Defense System Ia comprising a ZorA polypeptide, a ZorB polypeptide, a ZorC polypeptide, and a ZorD polypeptide, is encoded by a nucleic acid sequence having at least 85% identity, or 90% identity, or 95% identity, or 97% identity, or 98% identity, or 99% identity to the sequence set forth in SEQ ID NO: 14.

In some embodiments, the ZORYA anti-phage defense system comprises a nucleic acid construct comprising nucleic acid sequences encoding a cassette comprising zorA, zorB, and zorE genes. In some embodiments, a construct comprising the ZORYA defense system encodes one component of the defense system, whereby multiple constructs may be used to assemble the functional defense system. In some embodiments, the components of a ZORYA Type II defense system comprise genes zorA, zorB, and zorE. In some embodiments, the components of a ZORYA Type II defense system consist of genes zorA, zorB, and zorE. In some embodiments, the components of a ZORYA Type II defense system comprise nucleic acid sequences encoding a ZorA polypeptide, a ZorB polypeptide, and a ZorE polypeptide. In some embodiments, the components of a ZORYA Type II defense system consist of nucleic acid sequences encoding a ZorA polypeptide, a ZorB polypeptide, and a ZorE polypeptide.

In some embodiments, a Zorya Type II (Defense System Ib) gene cassette comprises the nucleic acid sequence:

(SEQ ID NO: 15; construct 51 from Table 4)

| | |
|---|---|
| 1 | acctgccttc ctttgataca attcgtaaca ggttactatc atcataaaaa agctcaaccc |
| 61 | gatgaactcg ctaaaaatga gacaaatcat ttatatctcg aaaaaacttg ttacaatcat |
| 121 | gagcgctaca ccgaacttaa ccatataaat tatgtgtgtt ttgtttattt tttaaacgat |
| 181 | tacaactatc cattatttac acaggtatca aaatgttagc gcagcttttt gagcagttgt |
| 241 | ttcaatcgat agactctaca ctgatcacca atattttcat ctgggctgtt atattcgtat |
| 301 | ttttatcagc gtggtggtgt gacaaaaaaa atatacatag taagtttaga gaatatgctc |
| 361 | caaccttaat gggggcatta ggtattctgg gtactttcat tggtattatt attggtttac |
| 421 | tcaattttaa taccgaaagt attgatacca gcatccccgt attattaggt ggcctaaaaa |
| 481 | cagcattcat tacaagcatt gtaggtatgt tttttgccat tttatttaat ggaatggatg |
| 541 | ctttcttttt tgccaataaa cgaagtgcgt tagctgaaaa taaccctgaa tctgttacac |
| 601 | ctgaacatat ctatcatgaa ttaaaagagc agaaccagac tctgactaaa ttagtctcgg |
| 661 | gtattaacgg tgatagtgaa ggttctctta ttgctcaaat aaaattacta cgtactgaga |
| 721 | ttagcgattc ctcgcaggca caattagcta atcacactca tttcagtaat aagctttggg |
| 781 | aacaacttga acaatttgca gatctaatgg caaaaggtgc tacagaacaa attattgatg |
| 841 | ctttgcgaca agtcattatt gattttaatg aaaatttaac tgaacagttt ggtgaaaact |
| 901 | ttaaagctct tgatgcctct gtaaaaaaac ttgttgagtg gcagggaaat tataaaacgc |
| 961 | aaattgagca gatgtcagaa caatatcaac aaagtgtcga gtccctggtt gaaacaaaaa |
| 1021 | ctgcggttgc agggatttgg gaagaatgta aagaaattcc tctggctatg tctgaactgc |
| 1081 | gtgaagtgct tcaggtgaac caacatcaaa tcagcgaact ctcccgccat ttagaaacct |
| 1141 | ttgtcgccat ccgcgataaa gctacaaccg tattacctga aatacagaac aaaatggctg |
| 1201 | aagtgggtga actgctgaaa tccggagctg caaatgttag tgcatctctt gagcaaacca |
| 1261 | gccagcaaat acttcttaat gcagattcaa tgcgcgttgc cctggatgaa ggtaccgaag |
| 1321 | gattcagaca atcggttacc caaacacaac aagcatttgc ctcgatggcg catgatgtca |
| 1381 | gcaattcctc cgaaacccta accagcacgt taggtgaaac aattactgaa atgaaacaaa |
| 1441 | gtggtgaaga attcctgaaa tcactagagt cgcactcgaa agaattgcat agaaatatgg |
| 1501 | aacaaaatac gacgaatgtg attgatatgt tcagtaagac tggtgaaaag attaaccatc |
| 1561 | aactatccag taatgccgat aatatgtttg attcaatcca gacatcattt gataaggctg |
| 1621 | gtgcagggct gacttctcaa gtcagagaat caattgaaaa atttgctcta tccatcaacg |
| 1681 | agcagttaca tgcttttgag caagcaactg aacgtgaaat gaaccgtgaa atgcaatcat |
| 1741 | taggtaatgc tctgctttca atcagcaaag gttttgtcgg taactatgaa aaacttatta |
| 1801 | aagattacca aatagttatg gggcagttac aagcattaat ttctgctaat aaacatcgag |
| 1861 | ggtaatcgat catggataag attataggga aacaattacc taaaaaagat caagataatg |
| 1921 | aacattgggt atccatgtca gacctaatgg cagggctgat gatggttttt atgttcatat |
| 1981 | ctattgctta tatgcactac gtacgtattg aaaaagaaaa aattaaagaa gttgccgtag |
| 2041 | cctacgagaa tgctcagtta cagatttata atgctctgga tattgagttt gcaaaggatt |
| 2101 | tacaagactg ggatgcagag atcgataaac agactctgga ggttcgattt aaatcaccgg |
| 2161 | atgttttatt tggcttagga agcacagagc taaaaccaaa gtttaaactc attcttgacg |
| 2221 | acttctttcc tcgctaccta aaagttctag ataattatca ggaacatatt actgaagtcc |
| 2281 | gcattgaagg tcacacaagt actgactgga caggaacaac gaatcctgat attgcttatt |
| 2341 | ttaataatat ggcactatcg caaggtcgta cacgtgcagt attacaatac gtttatgaca |
| 2401 | taaaaaatat cgcgacacac caacaatggg ttaaaagtaa atttgccgca gtaggttatt |
| 2461 | catctgcaca tcccattctt gataaaaccg gcaaagagga ccctaatcgc tctcgtcgtg |
| 2521 | tcaccttcaa agttgtaaca aatgccgagt tgcagattag aaagattatt caggagtaag |
| 2581 | agatgaaatt atctatcgac atttcagaac ttattcaatt agggaagaaa atgttaccag |
| 2641 | aaggagtcga tttttttctg gatgaatccc ctattgactt tgatcctata gatattgagt |
| 2701 | tatccacggg taaagaagtt agtatcgaag atcttgaccc tggtagcggg cttatctctt |
| 2761 | atcatggccg ccaggttctt ttatatattc gggaccattc agggcgttat gatgcggcta |
| 2821 | tcgtagatgg cgaaaaagga aaacgttttc atattgcctg gtgcagaact cttgatgaaa |
| 2881 | tgcgccataa aaatcgattt gaaaggtatc atgcaactaa ccgcatagat ggtttattcg |
| 2941 | aaattgatga tggttcaggt cggagccagg atgttgattt acgggtatgt atgaattgcc |
| 3001 | tcgaacgact taattataaa ggaagtattg ataaacaacg aaaaagagag atttttaaat |
| 3061 | cattctcatt aaatgagttt ttttcagatt atagtacctg ttttcgtcat atgcctaagg |
| 3121 | gtatctatga caaaacaaat agtgggtatg tcgaaaactg gaaggaaata tctaaagaaa |
| 3181 | tacgagaaaa ggcaaattat gtttgtaatg attgtggcgt gaatttatca accgccaaaa |
| 3241 | acttgtgcca tgtccatcat aaaaatggca tcaaatatga taatcaccat gaaaaccttc |
| 3301 | ttgttctgtg caaggattgc catcgaaaac agcccctcca cgaaggtata ttcgttaccc |
| 3361 | aagcagagat ggctatcatt caacgtttac gttcccaaca agggttatta aaagcagaat |
| 3421 | cctggaatga aatatatgac ctgactgatc catcagtgca tggtgatatt aatatgatgc |
| 3481 | aacataaagg ctttcaacct cctgttcctg ggttagatct tcaaaactca gaacatgaaa |
| 3541 | ttattgcaac cgtagaagct gcatggccag gccttaaaat tgcagttaac cttactcccg |
| 3601 | ccgaagtcga aggatggaga atatataccg tgggtgagct ggttaaagaa atacaaaccg |
| 3661 | gagcctttac gccagcaaaa ttgtaaattc taaaactccg tgaaagttaa ggctttcacg |
| 3721 | gaagataaat aaagtttccc tgatttgtga ctcaaattac aaaagtagtt tatggcataa |
| 3781 | cttgtctgat ttttatggtg taacaggtat aaaagcatat gctatggttc gcctcatact |
| 3841 | taaaacttcc ctcatatggg tgaaggttaa agcttggtag acagaagaca gtcacaatga |
| 3901 | ataaagcaat aaattga. |

The coding regions for each of the zorA, zorB, and zorE gene sequences within this embodiment of a Zorya Type II cassette (SEQ ID NO: 15) are as follows: nucleotides 213-1865 encode an embodiment of a ZorA polypeptide; nucleotides 1872-2579 encode an embodiment of a ZorB polypeptide; and nucleotides 2583-3686 encode an embodiment of a ZorE polypeptide.

In some embodiments, a Defense System Ib comprising a ZorA polypeptide, a ZorB polypeptide, and a ZorE polypeptide, is encoded by the nucleic acid sequence set forth in SEQ ID NO: 15. In some embodiments, a Defense System Ib comprising a ZorA polypeptide, a ZorB polypeptide, and a ZorE polypeptide, is encoded by a nucleic acid sequence having at least 80% homology to the sequence set forth in SEQ ID NO: 15. In some embodiments, a Defense System Ib comprising a ZorA polypeptide, a ZorB polypeptide, and a ZorE polypeptide, is encoded by a nucleic acid sequence having at least 85% homology, or 90% homology, or 95% homology, or 97% homology, or 98% homology, or 99% homology to the sequence set forth in SEQ ID NO: 15. In some embodiments, a Defense System Ib comprising a ZorA polypeptide, a ZorB polypeptide, and a ZorE polypeptide, is encoded by a nucleic acid sequence having at least 80% identity to the sequence set forth in SEQ ID NO: 15. In some embodiments, a Defense System Ib comprising a ZorA polypeptide, a ZorB polypeptide, and a ZorE polypeptide, is encoded by a nucleic acid sequence having at least 85% identity, or 90% identity, or 95% identity, or 97% identity, or 98% identity, or 99% identity to the sequence set forth in SEQ ID NO: 15.

In some embodiments, a construct comprising the ZORYA defense system encodes more than one component of the defense system but less than all of the components required for a functionally active defense system, whereby multiple constructs may be used to assemble the functional defense system. For example, in some embodiments each of ZorA, ZorB, ZorC, and ZorD may be encoded by nucleic acid sequences comprised in different constructs, wherein expression of the combination of constructs produces a functional ZORYA Type I defense system. In some embodiments each of ZorA, ZorB, and ZorE may be encoded by nucleic acid sequences comprised in different constructs, wherein expression of the combination of constructs produces a functional ZORYA Type II defense system.

In some embodiments, the components making up a functional ZORYA Type I anti-phage defense system comprise a ZorA polypeptide, a ZorB polypeptide, a ZorC polypeptide, and a ZorD polypeptide, each encoded by a zorA, a zorB, a zorC, and a zorD gene, respectively.

In some embodiments, the components making up a functional ZORYA Type II anti-phage defense system comprise a ZorA polypeptide, a ZorB polypeptide, and a ZorE polypeptide, each encoded by a zorA, a zorB, and a zorE gene, respectively.

In some embodiments, a ZORYA defense system having an anti-phage activity comprise a nucleic acid encoding a ZorA polypeptide. In some embodiments, a ZORYA defense system having an anti-phage activity comprise a nucleic acid encoding a ZorA polypeptide and a ZorB polypeptide. In some embodiments, a ZORYA defense system having an anti-phage activity comprise a nucleic acid encoding a ZorA polypeptide and a ZorC polypeptide. In some embodiments, a ZORYA defense system having an anti-phage activity comprise a nucleic acid encoding a ZorA polypeptide and a ZorD polypeptide. In some embodiments, a ZORYA defense system having an anti-phage activity comprise a nucleic acid encoding a ZorA polypeptide and a ZorE polypeptide. In some embodiments, a ZORYA defense system having an anti-phage activity comprise a nucleic acid encoding a ZorA polypeptide, a ZorB polypeptide, and a ZorC polypeptide. In some embodiments, a ZORYA defense system having an anti-phage activity comprise a nucleic acid encoding a ZorA polypeptide, a ZorB polypeptide, and a ZorD polypeptide. In some embodiments, a ZORYA defense system having an anti-phage activity comprise a nucleic acid encoding a ZorA polypeptide, a ZorC polypeptide, and a ZorD polypeptide. In some embodiments, a ZORYA defense system having an anti-phage activity comprise a nucleic acid encoding a ZorB polypeptide. In some embodiments, a ZORYA defense system having an anti-phage activity comprise a nucleic acid encoding a ZorB polypeptide and a ZorC polypeptide. In some embodiments, a ZORYA defense system having an anti-phage activity comprise a nucleic acid encoding a ZorB polypeptide and a ZorD polypeptide. In some embodiments, a ZORYA defense system having an anti-phage activity comprise a nucleic acid encoding a ZorB polypeptide and a ZorE polypeptide. In some embodiments, a ZORYA defense system having an anti-phage activity comprise a nucleic acid encoding a ZorC polypeptide. In some embodiments, a ZORYA defense system having an anti-phage activity comprise a nucleic acid encoding a ZorC polypeptide and a ZorD polypeptide. In some embodiments, a ZORYA defense system having an anti-phage activity comprise a nucleic acid encoding a ZorE polypeptide.

In some embodiments, a ZORYA defense system having an anti-phage activity comprise a nucleic acid comprising a zorA gene. In some embodiments, a ZORYA defense system having an anti-phage activity comprise a nucleic acid comprising a zorA gene and a zorB gene. In some embodiments, a ZORYA defense system having an anti-phage activity comprise a nucleic acid comprising a zorA gene and a zorC gene. In some embodiments, a ZORYA defense system having an anti-phage activity comprise a nucleic acid comprising a zorA gene and a zorD gene. In some embodiments, a ZORYA defense system having an anti-phage activity comprise a nucleic acid comprising a zorA gene and a zorE gene. In some embodiments, a ZORYA defense system having an anti-phage activity comprise a nucleic acid comprising a zorA gene, a zorB gene, and a zorC gene. In some embodiments, a ZORYA defense system having an anti-phage activity comprise a nucleic acid comprising a zorA gene, a zorB gene, and a zorD gene. In some embodiments, a ZORYA defense system having an anti-phage activity comprise a nucleic acid comprising a zorA gene, a zorC gene, and a zorD gene. In some embodiments, a ZORYA defense system having an anti-phage activity comprise a nucleic acid comprising a zorB gene. In some embodiments, a ZORYA defense system having an anti-phage activity comprise a nucleic acid comprising a zorB gene and a zorC gene. In some embodiments, a ZORYA defense system having an anti-phage activity comprise a nucleic acid comprising a zorB gene and a zorD gene. In some embodiments, a ZORYA defense system having an anti-phage activity comprise a nucleic acid comprising a zorB gene and a zorE gene. In some embodiments, a ZORYA defense system having an anti-phage activity comprise a nucleic acid comprising a zorC gene. In some embodiments, a ZORYA defense system having an anti-phage activity comprise a nucleic acid comprising a zorC gene and a zorD gene. In some embodiments, a ZORYA defense system having an anti-phage activity comprise a nucleic acid comprising a zorE gene.

In some embodiments, a ZORYA defense system having an anti-phage activity comprises a nucleic acid construct comprising nucleic acids encoding polypeptides in a set order. In one embodiment, the 5′ to 3′ order of polypeptides encoded is ZorA, ZorB, ZorC, and ZorD. In one embodiment, the 5′ to 3′ order of polypeptides encoded is ZorA, ZorB, and ZorE. In some embodiments the 5′ to 3′ order of polypeptides does not affect the anti-phage activity. In some embodiments the 5′ to 3′ order of polypeptides does affect the anti-phage activity.

In some embodiments, the 5′ to 3′ order of polypeptides is random, for example any order of ZorA, ZorB, ZorC, ZorD, and ZorD. In some embodiments, the 5′ to 3′ order of polypeptides is random, for example any order of ZorA, ZorB, and ZorE.

In some embodiments, a ZORYA defense system having an anti-phage activity comprises a nucleic acid construct comprising genes in a set order. In some embodiment, the 5′ to 3′ order of genes is zorA, zorB, zorC, and zorD (Defense System Ia). In some embodiment, the 5′ to 3′ order of genes is zorA, zorB, and zorE (Defense System Ib). In some embodiment, the 5′ to 3′ order of genes in a Defense System Ia is not zorA, zorB, zorC, and zorD. In some embodiment, the 5′ to 3′ order of genes in a Defense System Ib is not zorA, zorB, and zorE. In some embodiments the 5′ to 3′ order of genes does not affect the anti-phage activity. In some embodiments the 5′ to 3′ order of genes does affect the anti-phage activity.

In some embodiments, the 5′ to 3′ order of genes is random, for example any order of zorA, zorB, zorC, zorD, and zorD. In some embodiments, the 5′ to 3′ order of genes is random, for example any order of zorA, zorB, and zorE.

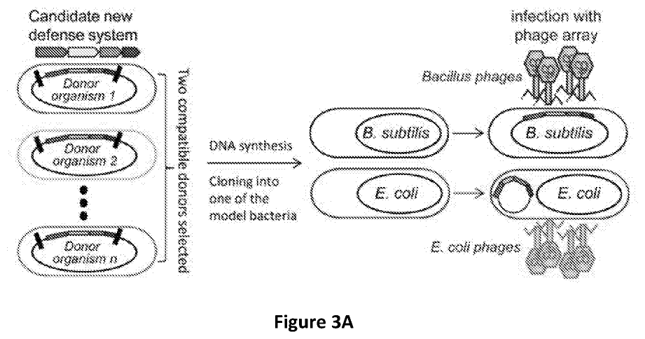

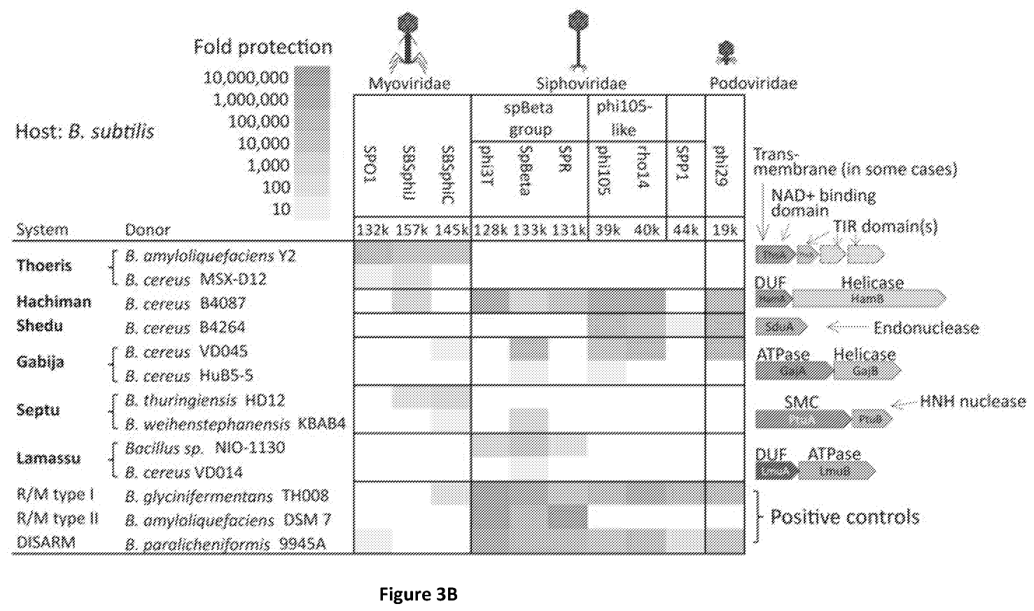

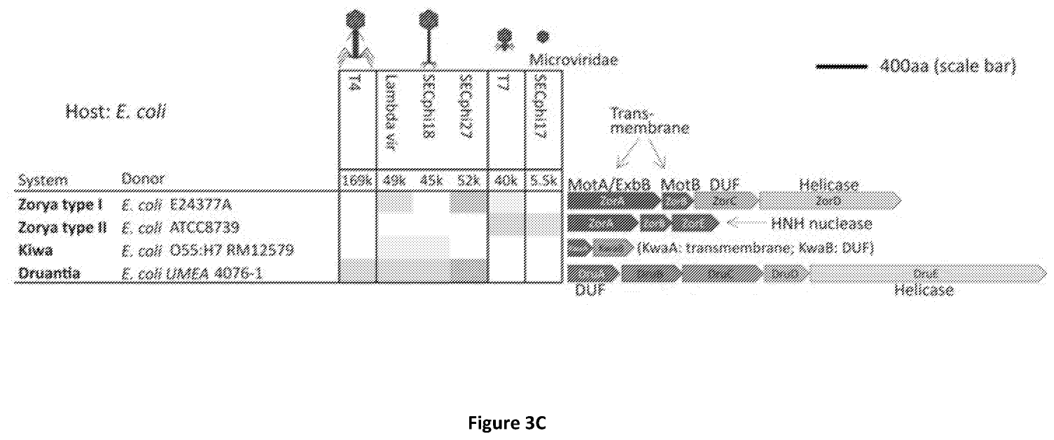

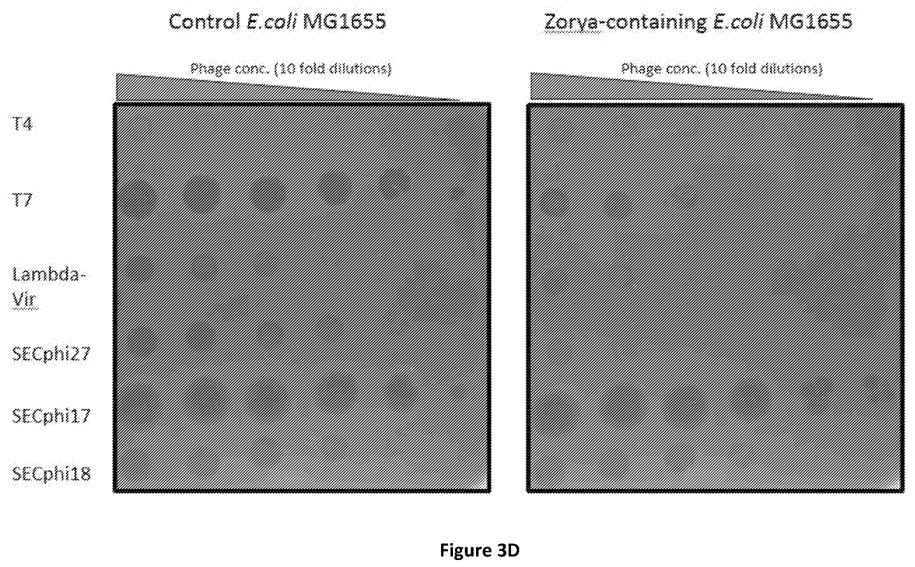

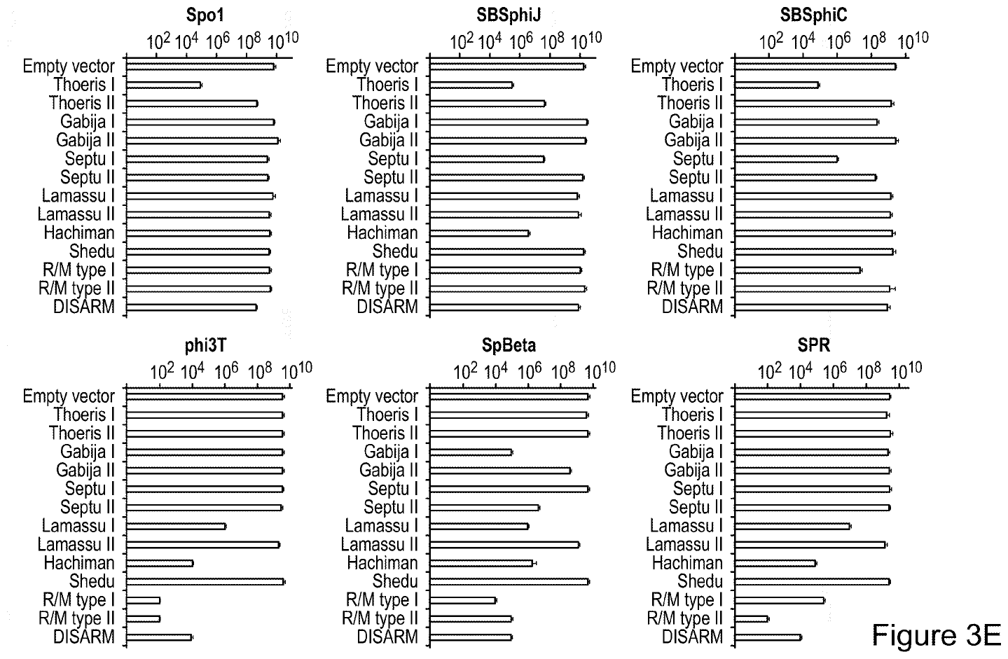

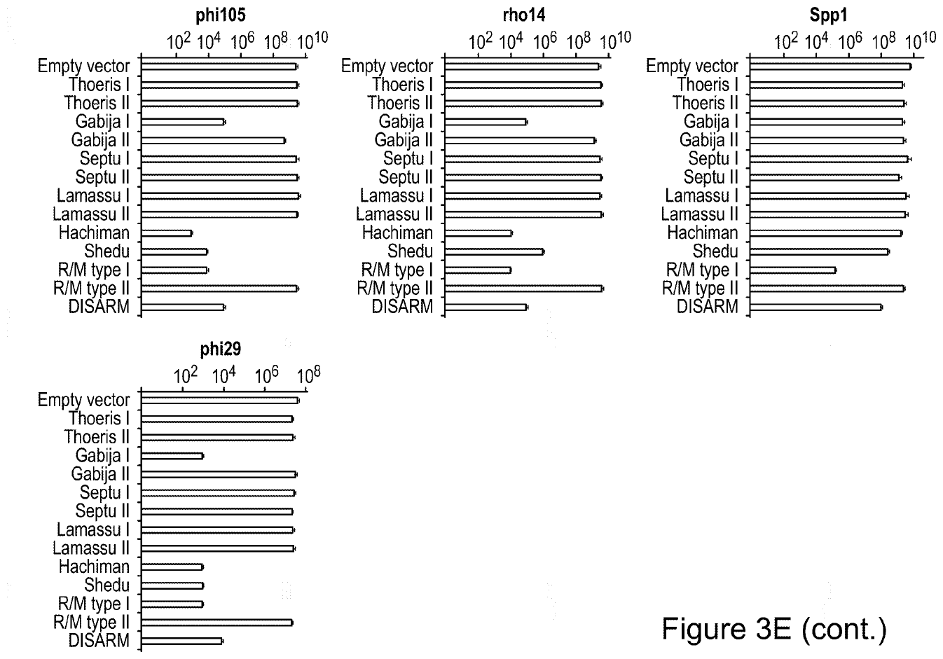

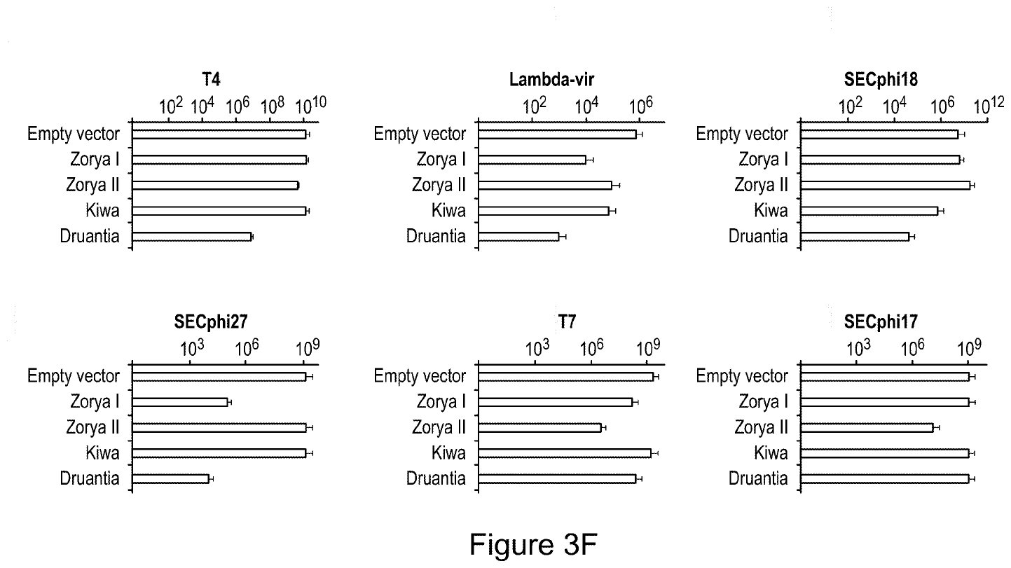

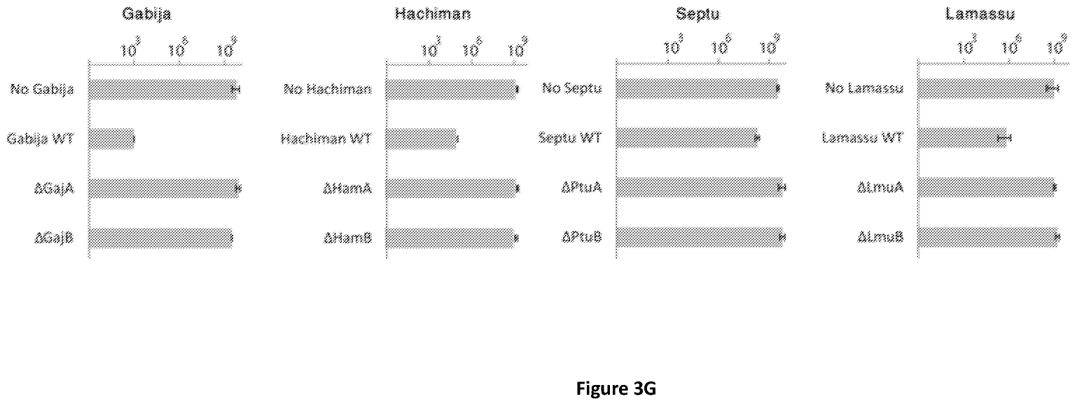

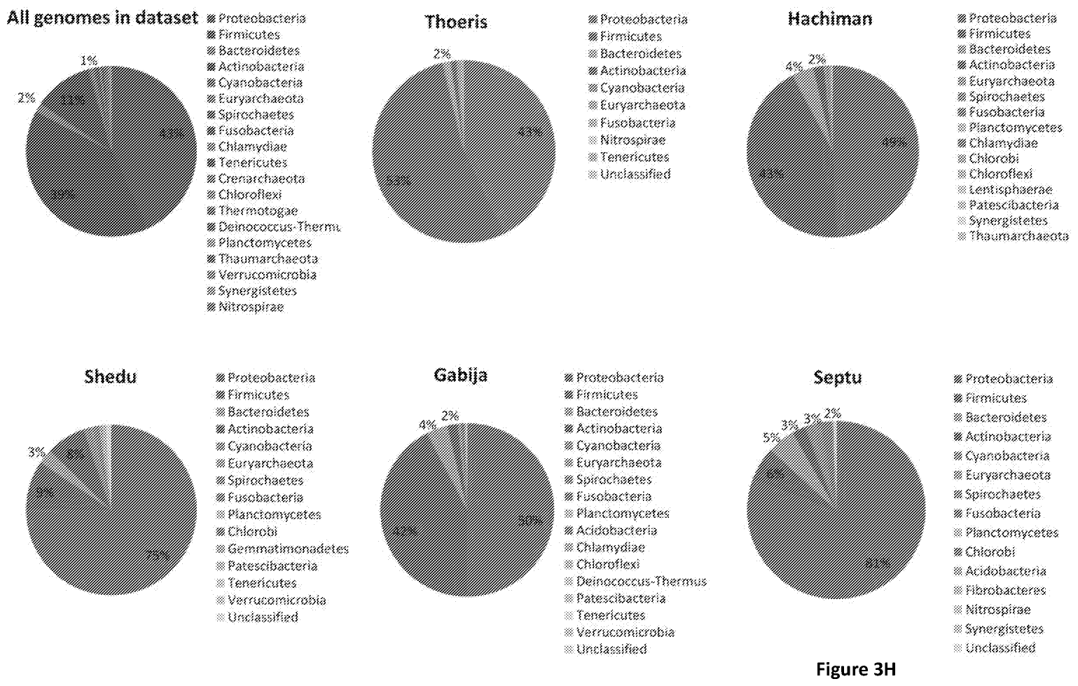

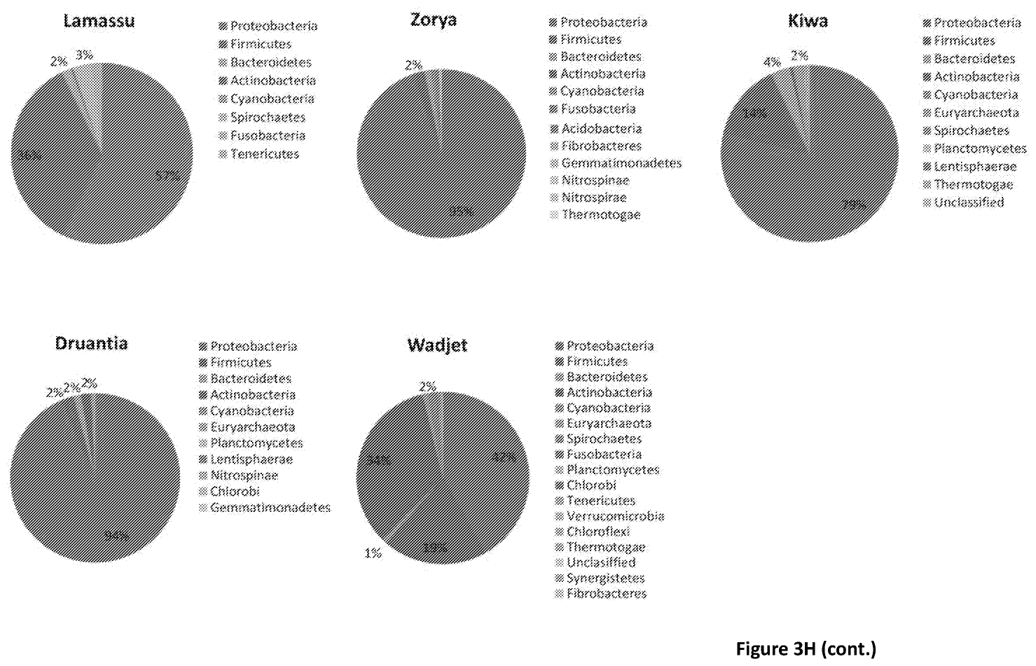

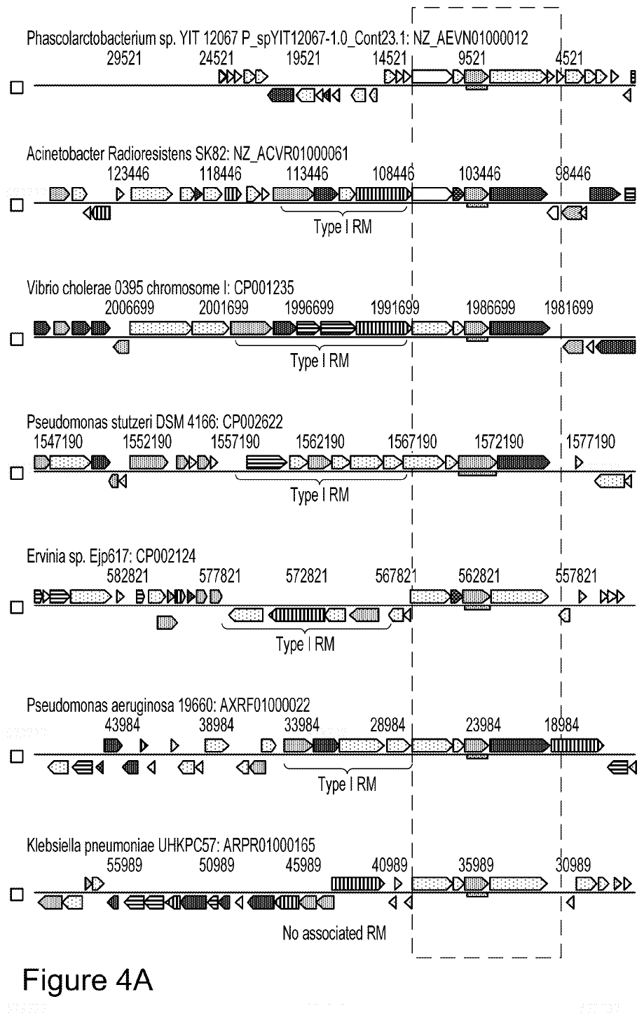

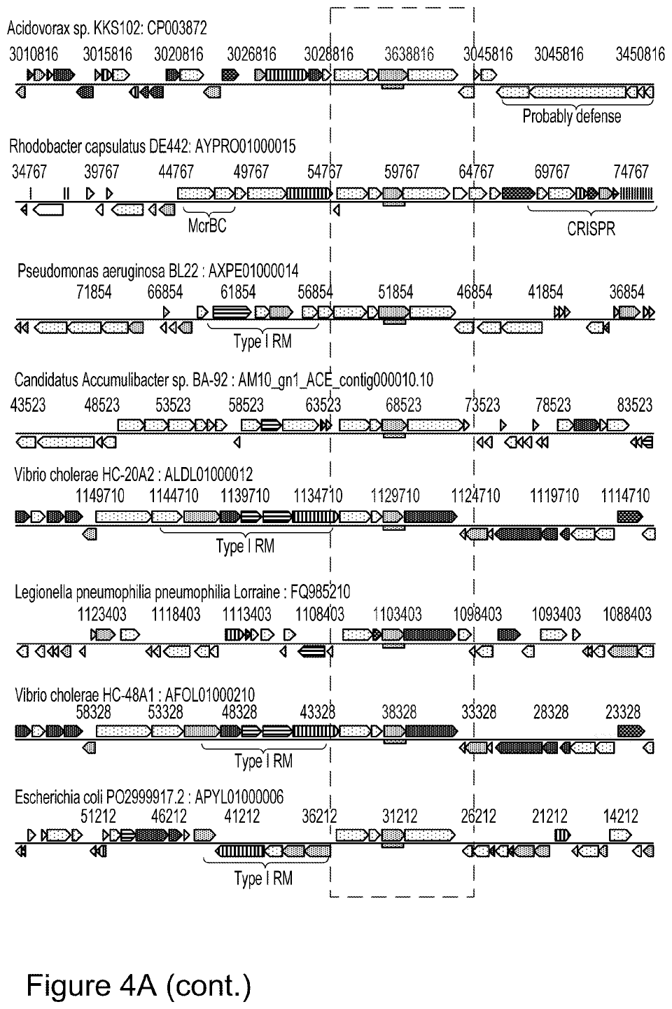

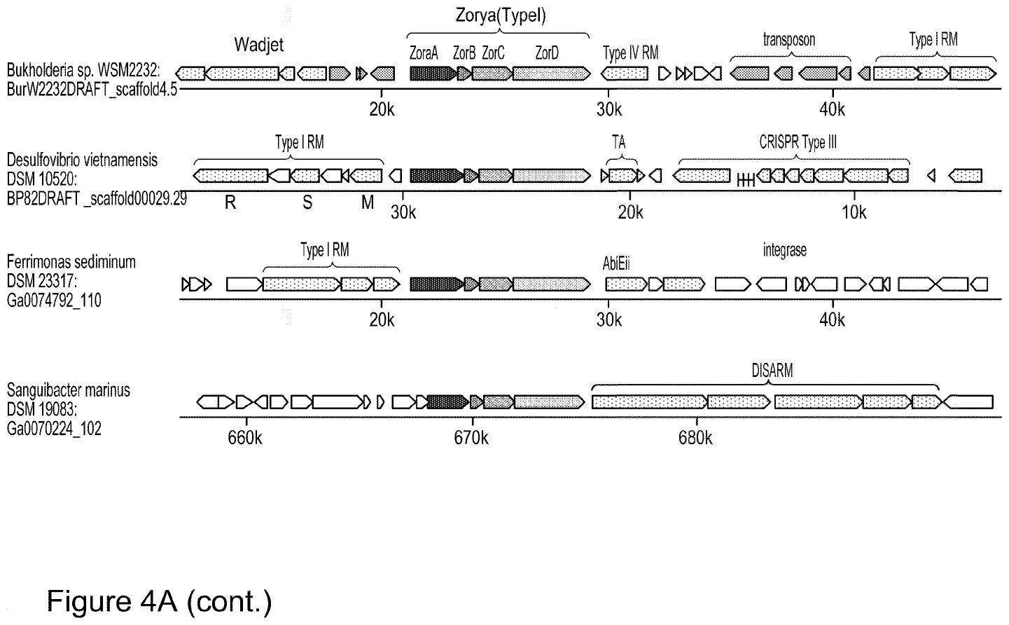

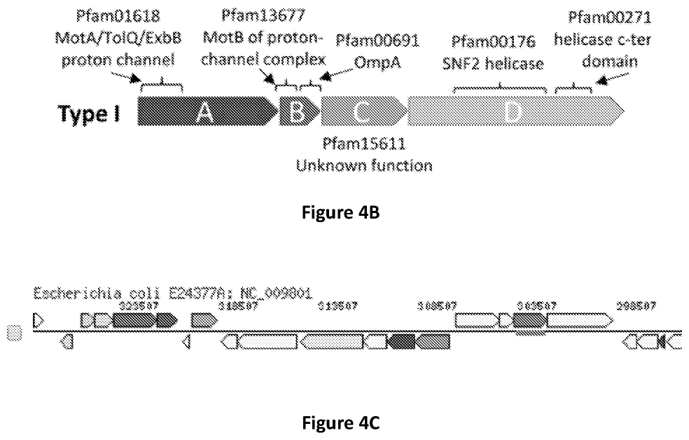

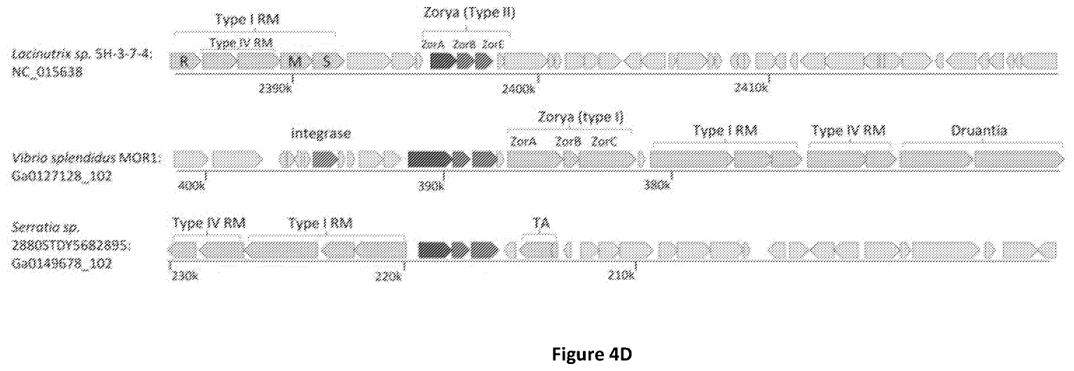

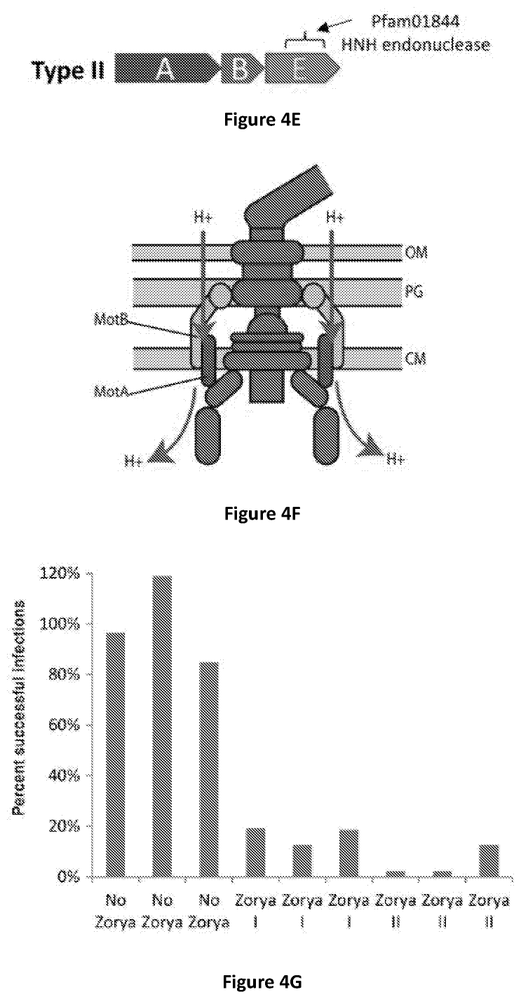

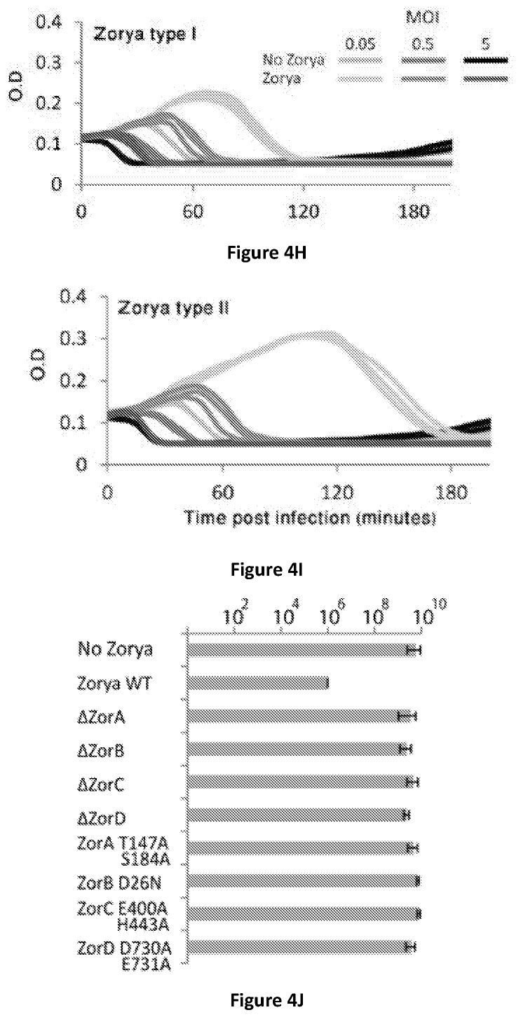

In some embodiments, the ZORYA Type I system (Defense System Ia) composition and order is as shown in FIG. 3C, FIG. 4A, and FIG. 4B. In some embodiments, the ZORYA Type II system (Defense System Ib) composition and order is as shown in FIG. 3C, FIG. 4D, and FIG. 4E.

In some embodiments, a ZORYA defense system having an anti-phage activity originates from a microbial genome (Table 8).

In some embodiments, a functional ZORYA defense system comprises a construct comprising nucleic acid sequences encoding polypeptides, wherein the nucleic acid sequence encoding each polypeptide may originate from a different microbial species. In some embodiments, a Zorya defense system (Defense system Ia; Defense system Ib) comprises a non-naturally occurring combination of polypeptide components. In some embodiments, a Zorya defense system (Defense system Ia; Defense system Ib) comprises a combination of at least two polypeptides that do not naturally occur together. In some embodiments, a Zorya defense system (Defense system Ia; Defense system Ib) comprises a combination of at least three polypeptides that do not naturally occur together. In some embodiments, a Zorya defense system (Defense system Ia) comprises a combination of four polypeptides that do not naturally occur together. In some embodiments, a Zorya defense system (Defense system Ia; Defense system Ib) comprises a non-naturally occurring combination of polypeptide components. In some embodiments, a functional Zorya defense system (Defense system Ia; Defense system Ib) comprises a combination of at least two polypeptides that do not naturally occur together. In some embodiments, a functional Zorya defense system (Defense system Ia; Defense system Ib) comprises a combination of at least three polypeptides that do not naturally occur together. In some embodiments, a functional Zorya defense system (Defense system Ia) comprises a combination of four polypeptides that do not naturally occur together.

A skilled artisan would appreciate that the term “non-naturally occurring” may encompass in one embodiment, polypeptide that do not naturally occur together in nature. For example, polypeptide components that do not naturally occur together, either because they are from the same defense system found in a different strain of bacteria or because they comprise polypeptide components from different defense systems. In some embodiments, polypeptide components that do not naturally occur together, either because they are from the same defense system found in a different strain of a microorganism, or because they comprise polypeptide components from different defense systems. In some embodiments, the term “non-naturally occurring” may encompass a defense system or a functional defense system that is not found within a particular micro-organism in nature. In some embodiments, the term “non-naturally occurring” may encompass a defense system or a functional defense system that is not found within a particular bacteria in nature. In some embodiments, the term “non-naturally occurring” may encompass a combination of defense systems disclosed herein or a functional defense system disclosed herein that are not found within a particular micro-organism in nature. In some embodiments, the term “non-naturally occurring” may encompass a combination of defense systems disclosed herein or a functional defense systems disclosed herein that are not found within a particular bacteria in nature.

In some embodiments, introducing a defense system disclosed herein, or any combination of defense systems, into a bacterial strain that naturally lacks the defense system or system may be advantageous for the bacteria. In some embodiments, introducing a defense system disclosed herein, or any combination of defense systems, into a bacterial strain that naturally lacks the defense system or system may be biotechnologically useful in maintaining healthy bacteria used for example but not limited to, the food industry.

In some embodiments, a functional ZORYA defense system comprises a construct comprising nucleic acid sequences encoding polypeptides, wherein the nucleic acid sequence encoding each polypeptide may originate from a different bacterial species. For example, the source of the nucleic acid sequence encoding a ZorA polypeptide may be one bacterial species from Table 8, the source of the nucleic acid sequence encoding a ZorB polypeptide may be a different bacterial species from Table 8, the source of the nucleic acid sequence encoding a ZorC polypeptide may be yet a different bacterial species from Table 8, and the source of the nucleic acid sequence encoding a ZorD polypeptide may be still a different bacterial species from Table 8. Similarly, the source of the nucleic acid sequence encoding a ZorA polypeptide may be one bacterial species from Table 8, the source of the nucleic acid sequence encoding a ZorB polypeptide may be a different bacterial species from Table 8, and the source of the nucleic acid sequence encoding a ZorE polypeptide may be still a different bacterial species from Table 8.

In some embodiments, the source of the nucleic acid encoding a ZorA polypeptide, a ZorB polypeptide, a ZorC polypeptide, and a ZorD polypeptide is the same. In some embodiments, the source of the nucleic acid encoding a ZorA polypeptide, a ZorB polypeptide, a ZorC polypeptide, and a ZorD polypeptide is the not the same. In some embodiments, the source of some of the components is the same, while the source of other components is not the same. In some embodiments, the source of the nucleic acid sequence of any of the components of a ZORYA Type I defense system comprises any of the species listed in Table 8.

In some embodiments, the source of the nucleic acid encoding a ZorA polypeptide, a ZorB polypeptide, and a ZorE polypeptide is the same. In some embodiments, the source of the nucleic acid encoding a ZorA polypeptide, a ZorB polypeptide, and a ZorE polypeptide is the not the same. In some embodiments, the source of some of the components is the same, while the source of other components is not the same. In some embodiments, the source of the nucleic acid sequence of any of the components of a ZORYA Type II defense system comprises any of the species listed in Table 8.

In some embodiments, a ZORYA defense system having an anti-phage activity comprises a nucleic acid construct comprising non-coding regions between the nucleic acid sequences encoding the polypeptides. In some embodiments, the non-coding regions between the nucleic acid sequences comprises nucleic acid sequence not naturally occurring in the microbial species of origin. In some embodiments, the non-coding regions between the nucleic acid sequences comprises nucleic acid sequence is the naturally occurring in the microbial species of origin.

In some embodiments, a ZORYA system disclosed herein comprises a multi-gene phage resistance system broadly distributed in microbial genomes, for example but not limited to bacteria. A skilled artisan would appreciate that the ZORYA system is not present in the majority of bacterial species.

According to some embodiments, the ZORYA system components are located in a gene cluster in a microbial cell genome. According to some embodiments, the ZORYA system components are located in close proximity (e.g. positioned within 20 genes, 10 genes, 5 genes or less one from the other) in a genome of a prokaryotic cell. According to some embodiments the prokaryotic cell expresses an endogenous ZORYA defense system. According to some embodiments, the prokaryotic cell expresses an endogenous functional ZORYA defense system. According to some embodiments, the species of prokaryotic cell is selected from the group consisting of the species listed in Table 8. According to some embodiments, a prokaryotic cell expresses a non-endogenous ZORYA defense system. According to some embodiments, a prokaryotic cell expresses a non-endogenous functional ZORYA defense system. According to some embodiments, the species of prokaryotic cell expressing a non-endogenous functional ZORYA defense system is selected from the group consisting of the species listed in Table 8.

A skilled artisan would appreciate that the term “endogenous” may encompass the expression of a native gene in its natural location and with its natural expression level in the genome of an organism, for example a prokaryote. In some embodiments, an endogenous anti-nucleic acid defense system is found in bacterial or in archaeal cells. In some embodiments, an endogenous anti-nucleic acid defense system is found in bacterial cells. In some embodiments, an endogenous anti-nucleic acid defense system is found in archaeal cells.

In some embodiments, a ZORYA Type I defense system (Defense System Ia) components comprise ZorA, ZorB, ZorC and ZorD polypeptides. In some embodiments, a ZORYA Type I defense system components comprise functional portions of ZorA, ZorB, ZorC and ZorD polypeptides. In some embodiments, the ZORYA Type I defense system components are encoded by zorA, zorB, zorC and zorD genes.

In some embodiments, a ZORYA Type II defense system (Defense System Ib) components comprise ZorA, ZorB, and ZorE polypeptides. In some embodiments, a ZORYA Type II defense system components comprise functional portions of ZorA, ZorB, and ZorE polypeptides. In some embodiments, the ZORYA Type II defense system components are encoded by zorA, zorB, and zorE genes.

Non-limiting embodiments of endogenous ZORYA systems and the respective location of their components are provided in Table 8 herein.

In some embodiments, the components of a ZORYA system may be identified by the presence of similar domains present within each component. In some embodiments, domains comprise pfam domains and or clusters of orthologous groups (COG) domains.

A skilled artisan would appreciate that the term “pfam” may encompass a large collection of protein domains and protein families maintained by the pfam consortium and available at several sponsored world wide web sites, including for example: pfam.sanger.ac.uk/ (Welcome Trust, Sanger Institute); pfam.sbc.su.se/ (Stockholm Bioinformatics Center); pfam(dot)janelia(dot)org/ (Janelia Farm, Howard Hughes Medical Institute); pfam(dot)jouy(dot)inra(dot)fr/ (Institut national de la Recherche Agronomique); and pfam.ccbb.re.kr/. pfam domains and families are identified using multiple sequence alignments and hidden Markov models (HMMs) (see e.g. R. D. Finnet et al. nucleic Acids Research Database (2010) Issue 38: D211-222). By accessing the pfam database, for example, using any of the above-reference websites, protein sequences can be queried against the hidden Markov models (HMMs) using HMMER homology search software (e.g., HMMER3, hmmer(dot)janelia(dot)org/).

A skilled artisan would appreciate that the term “COG (clusters of orthologous groups)” may encompass a large collection of protein families classified according to their homologous relationships available at e.g. the NCBI COG website (http://www(dot)ncbi(dot)nlm(dot)nih(dot)gov/COG). Each COG consists of a group of proteins found to be orthologous across at least three lineages and likely corresponds to an ancient conserved domain [see e.g. Tatusov et al. Science 1997 Oct. 24; 278(5338):631-7; and Tatusov et al. nucleic Acids Res. 2000 Jan. 1; 28(1): 33-36].

In some embodiments, a Defense System Ia comprises a membrane associated complex. In some embodiments, a Defense System Ib comprises a membrane associated complex.

In some embodiments, the term “ZorA” refers to the polynucleotide or expression product e.g., the polypeptide encoded by the zorA gene. In some embodiments, the term “ZorA” refers to a ZorA polypeptide. In some embodiments, the zorA gene encodes a polypeptide comprising a pfam01618 domain. In some embodiments, the zorA gene encodes a polypeptide comprising a COG0840 domain. In some embodiments, the zorA gene encodes a polypeptide comprising a pfam01618 domain and a COG0840 domain. In some embodiments, the ZorA polypeptide comprises a pfam01618 domain. In some embodiments, the ZorA polypeptide comprises a COG0840 domain. In some embodiments, the ZorA polypeptide comprises a pfam01618 domain and a COG0840 domain.

In some embodiments, the zorA gene encodes a polypeptide member of the MotA/TolQ/EXaB proton channel family. In some embodiments, the zorA gene encodes a polypeptide comprising 3 transmembrane helices.

In some embodiments, the ZorA polypeptide comprises a member of the MotA/TolQ/EXaB proton channel family. In some embodiments, the ZorA polypeptide comprises a member of a family comprising 3 transmembrane helices.

In some embodiments, ZorA polypeptide is encoded by a gene positioned within 5-60 genes of a gene encoding a ZorB, ZorC, ZorD, and or a ZorE polypeptide in a genome of a prokaryotic cell. In some embodiments, ZorA polypeptide is encoded by a gene positioned within 5 genes upstream (5′) or downstream (3′) to a gene encoding a ZorB, ZorC, ZorD, and/or a ZorE polypeptide in a genome of a prokaryotic cell. In some embodiments, ZorA polypeptide is encoded by a gene positioned within 5 genes upstream (5′) and downstream (3′) to a gene encoding a ZorB, ZorC, ZorD, and or ZorE polypeptide, or any combination thereof, in a genome of a prokaryotic cell.

In some embodiments, ZorA and ZorB are encoded by genes positioned sequentially 5′ to 3′ in a genome of a prokaryotic cell. In some embodiments, ZorA and ZorB are encoded by genes positioned contiguously 5′ to 3′ in a genome of a prokaryotic cell. In some embodiments, ZorA, ZorB, and ZorC are encoded by genes positioned sequentially 5′ to 3′ in a genome of a prokaryotic cell. In some embodiments, ZorA, ZorB, and ZorC are encoded by genes positioned contiguously 5′ to 3′ in a genome of a prokaryotic cell. In some embodiments, ZorA, ZorB, and ZorE are encoded by genes positioned sequentially 5′ to 3′ in a genome of a prokaryotic cell. In some embodiments, ZorA, ZorB, and ZorE are encoded by genes positioned contiguously 5′ to 3′ in a genome of a prokaryotic cell. In some embodiments, ZorA, ZorB, ZorC, and ZorD are encoded by genes positioned sequentially 5′ to 3′ in a genome of a prokaryotic cell. In some embodiments, ZorA, ZorB, ZorC, and ZorD are encoded by genes positioned contiguously 5′ to 3′ in a genome of a prokaryotic cell.

In some embodiments, zorA and zorB genes are positioned sequentially 5′ to 3′ in a genome of a prokaryotic cell. In some embodiments, zorA, and zorB, genes are positioned contiguously 5′ to 3′ in a genome of a prokaryotic cell. In some embodiments, zorA, zorB, and zorC genes are positioned sequentially 5′ to 3′ in a genome of a prokaryotic cell. In some embodiments, zorA, zorB, and zorC genes are positioned contiguously 5′ to 3′ in a genome of a prokaryotic cell. In some embodiments, zorA, zorB, and zorE genes are positioned sequentially 5′ to 3′ in a genome of a prokaryotic cell. In some embodiments, zorA, zorB, and zorE genes are positioned contiguously 5′ to 3′ in a genome of a prokaryotic cell. In some embodiments, zorA, zorB, zorC, and zorD genes are positioned sequentially 5′ to 3′ in a genome of a prokaryotic cell. In some embodiments, zorA, zorB, zorC, and zorD genes are positioned contiguously 5′ to 3′ in a genome of a prokaryotic cell.

In some embodiments, a ZorA polypeptide is about 550 amino acids long (Median gene size).

In some embodiments, the ZorA polypeptide comprises the amino acid sequence having at least 80% homology to a sequence selected from the group consisting the polypeptides referenced in Table 8, rows 2-1830 columns J and K. In some embodiments, the ZorA polypeptide comprises the amino acid sequence having at least 80% homology within similar domain regions within a sequence selected from the group consisting of the polypeptides referenced in Table 8, rows 2-1830 columns J and K.

A skilled artisan would appreciate that the terms “similar domain regions” encompasses but is not limited to pfam domains, COG domains, helices, membrane binding domains, transmembrane domains, structural domains, and enzyme active site domains.

In some embodiments, the ZorA polypeptide comprises a homolog of a polypeptide having the amino acid sequences set forth in the polypeptides referenced in Table 8, rows 2-1830 columns J and K. In some embodiments, a homolog of a ZorA polypeptide comprises a member of the MotA/TolQ/EXaB proton channel family. In some embodiments, a homolog of ZorA polypeptide comprises 3 transmembrane helices. In some embodiments, the ZorA polypeptide comprises the amino acid sequence having at least 80% identity to a sequence selected from the group consisting of the polypeptides referenced in Table 8, rows 2-1830 columns J and K.

A skilled artisan would appreciate that homology encompasses similarity of sequence attributed to descent from a common ancestor. Homologous biological components (genes, proteins, structures) are called homologs. The extent to which nucleotide or protein sequences are related. The similarity between two sequences (DNA, RNA, or amino acid) can be expressed as percent sequence identity and/or percent positive substitutions.

A skilled artisan would appreciate that the term “homolog” encompasses a gene or a polypeptide (a protein) that is related to a second gene or polypeptide (protein), respectively, by descent from a common ancestral DNA or polypeptide (protein) sequence, respectively. Thus, a homolog of a gene, in some embodiments, comprises a similar nucleotide sequence to the gene. In some embodiments, a gene homolog encodes an identical polypeptide as is encoded by the gene. In some embodiments, a gene homolog encodes a polypeptide with the same functional properties as is encoded by the gene. In some embodiments, a gene homolog encodes a polypeptide that comprises a similar amino acid sequence as the polypeptide encoded by the gene. In one embodiment, the polypeptide homolog comprises a similar amino acid sequence as the polypeptide. In some embodiments, the polypeptide homolog comprises the same functional properties as the polypeptide. In some embodiments, the polypeptide homolog comprises similar functional properties as the polypeptide. In some embodiments, the polypeptide homolog comprises a same domain(s) as the polypeptide. In some embodiments, the polypeptide homolog comprises a similar domain(s) as the polypeptide.

A skilled artisan would appreciate that percent homology or percent identity may be determined, for example but no limited to, using BlastP software of the National Center of Biotechnology Information (NCBI) using default parameters. The homolog may also refer to an ortholog, a deletion, insertion, or substitution variant, including an amino acid substitution. In some embodiments, sequence identity or homology can be determined using any protein or nucleic acid sequence alignment algorithm such as Blast, ClustalW, MUSCLE, and HHpred.

In some embodiments, gene homology and/or polypeptide homology can be based on shared motifs, for example but not limited to pfam domains, COG domains, DUF domains, transmembrane domains, and nuclease domains. In some embodiments, similarities of shared motifs are combined with the conserved size of the gene or the expression product in the different subtypes and the location of the gene in the gene cluster.

In some embodiments, the ZorA polypeptide comprises an amino acid sequence having at least 80%, at least 85%, at least 90%, or at least 95% homology to a sequence selected from the group consisting of the polypeptides referenced in Table 8, rows 2-1830 columns J and K. In some embodiments, the ZorA polypeptide comprises the amino acid sequence having at least 80%, at least 85%, at least 95% homology within similar domain regions within a sequence selected from the group consisting of the polypeptides referenced in Table 8, rows 2-1830 columns J and K. In some embodiments, the ZorA polypeptide comprises an amino acid sequence having at least 80%, at least 85%, at least 90%, or at least 95% identity to a sequence selected from the group consisting of the polypeptides referenced in Table 8, rows 2-1830 columns J and K. In some embodiments, the ZorA polypeptide comprises the amino acid sequence having at least 80%, at least 85%, at least 95% identity within similar domain regions within a sequence selected from the group consisting of the polypeptides referenced in Table 8, rows 2-1830 columns J and K. In some embodiments, the ZorA polypeptide comprises an amino acid sequence selected from the group consisting of the polypeptides referenced in Table 8, rows 2-1830 columns J and K.

In some embodiments, the ZorA polypeptide comprises an amino acid sequence encoded by a nucleic acid sequence having at least 80%, at least 85%, at least 90%, or at least 95% homology to a sequence selected from the group consisting of the polynucleotides referenced in Table 8, rows 2-1830 columns J and L. In some embodiments, the ZorA polypeptide comprises the amino acid sequence encoded by a nucleic acid sequence having at least 80%, at least 85%, at least 95% homology within similar domain regions within a sequence selected from the group consisting of the polynucleotides referenced in Table 8, rows 2-1830 columns J and L. In some embodiments, the ZorA polypeptide comprises an amino acid sequence encoded by a nucleic acid sequence having at least 80%, at least 85%, at least 90%, or at least 95% identity to a sequence selected from the group consisting of the polynucleotides referenced in Table 8, rows 2-1830 columns J and L. In some embodiments, the ZorA polypeptide comprises the amino acid sequence encoded by a nucleic acid sequence having at least 80%, at least 85%, at least 95% identity within similar domain regions within a sequence selected from the group consisting of the polynucleotides referenced in Table 8, rows 2-1830 columns J and L. In some embodiments, the ZorA polypeptide comprises an amino acid sequence encoded from a nucleic acid sequence selected from the group consisting of the polynucleotides referenced in Table 8, rows 2-1830 columns J and L.

In some embodiments, the nucleic acid sequence of a zorA gene comprises a nucleic acid sequence having at least 80%, at least 85%, at least 90%, or at least 95% homology to a sequence selected from the group consisting of the polynucleotides referenced in Table 8, rows 2-1830 columns J and L. In some embodiments, the nucleic acid sequence of a zorA gene comprises a nucleic acid sequence having at least 80%, at least 85%, at least 90%, or at least 95% identity to a sequence selected from the group consisting of the polynucleotides referenced in Table 8, rows 2-1830 columns J and L. In some embodiments, the nucleic acid sequence of a zorA gene comprises a nucleic acid sequence having at least 80%, at least 85%, at least 90%, or at least 95% homology within the nucleotide sequence encoding similar domain regions as a sequence selected from the group consisting of the polynucleotides referenced in Table 8, rows 2-1830 columns J and L. In some embodiments, the nucleic acid sequence of a zorA gene comprises a nucleic acid sequence having at least 80%, at least 85%, at least 90%, or at least 95% identity within the nucleotide sequence encoding similar domain regions as a sequence selected from the group consisting of the polynucleotides referenced in Table 8, rows 2-1830 columns J and L. In some embodiments, the nucleic acid sequence of a zorA gene comprises a nucleic acid sequence selected from the group consisting of the polynucleotides referenced in Table 8, rows 2-1830 columns J and L.

As used herein, the term “ZorB” refers to the polynucleotide or expression product e.g., polypeptide encoded by the zorB gene. In some embodiments, the term “ZorB” refers to a ZorB polypeptide. In some embodiments, the product of the zorB gene comprises a pfam13677 domain. In some embodiments, the product of the zorB gene comprises a pfam00691 domain. In some embodiments, the product of the zorB gene comprises a pfam13677 domain and a pfam00691 domain. In some embodiments, the zorB gene encodes a member of MotB proton channel family. In some embodiments, the zorB gene encodes a polypeptide comprising a transmembrane helix.

In some embodiments, ZorB polypeptide is encoded by a gene positioned within 5-60 genes of a gene encoding a ZorA, ZorC, ZorD, and or a ZorE polypeptide in a genome of a prokaryotic cell. In some embodiments, ZorB polypeptide is encoded by a gene positioned within 5 genes upstream (5′) or downstream (3′) to a gene encoding a ZorA, ZorC, ZorD, and/or a ZorE polypeptide in a genome of a prokaryotic cell.

In some embodiments, ZorA and ZorB are encoded by genes positioned sequentially 5′ to 3′ in a genome of a prokaryotic cell. In some embodiments ZorA and ZorB are encoded by genes positioned contiguously 5′ to 3′ in a genome of a prokaryotic cell. In some embodiments, ZorB and ZorC are encoded by genes positioned sequentially 5′ to 3′ in a genome of a prokaryotic cell. In some embodiments ZorB and ZorC are encoded by genes positioned contiguously 5′ to 3′ in a genome of a prokaryotic cell. In some embodiments, ZorB and ZorE are encoded by genes positioned sequentially 5′ to 3′ in a genome of a prokaryotic cell. In some embodiments ZorB and ZorE are encoded by genes positioned contiguously 5′ to 3′ in a genome of a prokaryotic cell. In some embodiments, ZorB, ZorC, and ZorD are encoded by genes positioned sequentially 5′ to 3′ in a genome of a prokaryotic cell. In some embodiments, ZorB, ZorC, and ZorD are encoded by genes positioned contiguously 5′ to 3′ in a genome of a prokaryotic cell.

In some embodiments, ZorB polypeptide is encoded by a gene positioned within 5 genes upstream or downstream to a gene encoding zorA, zorC, and zorD, in a genome of a prokaryotic cell. In some embodiments, ZorB polypeptide is encoded by a gene positioned within 5 genes upstream or downstream to a gene encoding zorA and zorE, in a genome of a prokaryotic cell.

In some embodiments, a ZorB polypeptide is about 217 amino acids long (median gene size).

In some embodiments, the ZorB polypeptide comprises an amino acid sequence having at least 80% homology to a sequence selected from the group consisting of the polypeptides referenced in Table 8, rows 2-1830 columns N and O.

In some embodiments, the ZorB polypeptide comprises the amino acid sequence having at least 80% homology within similar domain regions within a sequence selected from the group consisting of the polypeptides referenced in Table 8, rows 2-1830 columns N and O. In some embodiments, a homology of the ZorB polypeptide comprises a MotB proton channel family member. In some embodiments, the ZorB polypeptide comprises an amino acid sequence having at least 80% identity to a sequence selected from the group consisting of the polypeptides referenced in Table 8, rows 2-1830 columns N and O.

In some embodiments, the ZorB polypeptide comprises an amino acid sequence having at least 80%, at least 85%, at least 90%, or at least 95% homology to a sequence selected from the group consisting of the polypeptides referenced in Table 8, rows 2-1830 columns N and O. In some embodiments, the ZorB polypeptide comprises the amino acid sequence having at least 80%, at least 85%, at least 95% homology within similar domain regions within a sequence selected from the group consisting of the polypeptides referenced in Table 8, rows 2-1830 columns N and O. In some embodiments, the ZorB polypeptide comprises an amino acid sequence having at least 80%, at least 85%, at least 90%, or at least 95% identity to a sequence selected from the group consisting of the polypeptides referenced in Table 8, rows 2-1830 columns N and O. In some embodiments, the ZorB polypeptide comprises the amino acid sequence having at least 80%, at least 85%, at least 95% identity within similar domain regions within a sequence selected from the group consisting of the polypeptides referenced in Table 8, rows 2-1830 columns N and O. In some embodiments, the ZorB polypeptide comprises an amino acid sequence selected from the group consisting of the polypeptides referenced in Table 8, rows 2-1830 columns N and O.

In some embodiments, the ZorB polypeptide comprises an amino acid sequence encoded from a nucleic acid sequence having at least 80%, at least 85%, at least 90%, or at least 95% homology to a sequence selected from the group consisting of the polynucleotides referenced in Table 8, rows 2-1830 columns N and P. In some embodiments, the ZorB polypeptide comprises the amino acid sequence encoded by a nucleic acid sequence having at least 80%, at least 85%, at least 95% homology within similar domain regions within a sequence selected from the group consisting of the polynucleotides referenced in Table 8, rows 2-1830 columns N and P. In some embodiments, the ZorB polypeptide comprises an amino acid sequence encoded from a nucleic acid sequence having at least 80%, at least 85%, at least 90%, or at least 95% identity to a sequence selected from the group consisting of the polynucleotides referenced in Table 8, rows 2-1830 columns N and P. In some embodiments, the ZorB polypeptide comprises the amino acid sequence encoded by a nucleic acid sequence having at least 80%, at least 85%, at least 95% identity within similar domain regions within a sequence selected from the group consisting of the polynucleotides referenced in Table 8, rows 2-1830 columns N and P. In some embodiments, the ZorB polypeptide comprises an amino acid sequence encoded from a nucleic acid sequence selected from the group consisting of the polynucleotides referenced in Table 8, rows 2-1830 columns N and P.

In some embodiments, the nucleic acid sequence of a zorB gene comprises a nucleic acid sequence having at least 80%, at least 85%, at least 90%, or at least 95% homology to a sequence selected from the group consisting of the polynucleotides referenced in Table 8, rows 2-1830 columns N and P. In some embodiments, the nucleic acid sequence of a zorB gene comprises a nucleic acid sequence having at least 80%, at least 85%, at least 90%, or at least 95% identity to a sequence selected from the group consisting of the polynucleotides referenced in Table 8, rows 2-1830 columns N and P. In some embodiments, the nucleic acid sequence of a zorB gene comprises a nucleic acid sequence having at least 80%, at least 85%, at least 90%, or at least 95% homology within the nucleotide sequence encoding similar domain regions as a sequence selected from the group consisting of the polynucleotides referenced in Table 8, rows 2-1830 columns N and P. In some embodiments, the nucleic acid sequence of a zorB gene comprises a nucleic acid sequence having at least 80%, at least 85%, at least 90%, or at least 95% identity within the nucleotide sequence encoding similar domain regions as a sequence selected from the group consisting of the polynucleotides referenced in Table 8, rows 2-1830 columns N and P. In some embodiments, the nucleic acid sequence of a zorB gene comprises a nucleic acid sequence selected from the group consisting of the polynucleotides referenced in Table 8, rows 2-1830 columns N and P.

As used herein, the term “ZorC” refers to the polynucleotide or expression product e.g., polypeptide encoded by the zorC gene. In some embodiments, the term “ZorC” refers to a ZorC polypeptide. In some embodiments, the product of a zorC gene comprises a pfam15611 domain. In some embodiments, the product of a zorC gene comprises a EH signature domain.

In some embodiments, ZorC polypeptide is encoded by a gene positioned within 5-60 genes of a gene encoding ZorA, ZorB and/or ZorD in a genome of a prokaryotic cell. In some embodiments, ZorC polypeptide is encoded by a gene positioned within 5 genes upstream (5′) or downstream (3′) to a gene encoding ZorA, ZorB and/or ZorD in a genome of a prokaryotic cell. In some embodiments, ZorC polypeptide is encoded by a gene positioned within 5 genes upstream and downstream to a gene encoding ZorA, ZorB and ZorD in a genome of a prokaryotic cell.

In some embodiments, ZorC and ZorD are encoded by genes positioned sequentially 5′ to 3′ in a genome of a prokaryotic cell. In some embodiments, ZorC and ZorD are encoded by genes positioned contiguously 5′ to 3′ in a genome of a prokaryotic cell.

In some embodiments, zorC and zorD genes are positioned sequentially 5′ to 3′ in a genome of a prokaryotic cell. In some embodiments, zorC and zorD genes are positioned contiguously 5′ to 3′ in a genome of a prokaryotic cell.

In some embodiments, a ZorC polypeptide is about 470 amino acids long (median gene size).

In some embodiments, the ZorC polypeptide comprises an amino acid sequence having at least 80% homology to a sequence selected from the group consisting of the polypeptides referenced in Table 8, rows 2-1174 columns R and S. In some embodiments, the ZorC polypeptide comprises the amino acid sequence having at least 80% homology within similar domain regions within a sequence selected from the group consisting of the polypeptides referenced in Table 8, rows 2-1174 columns R and S. In some embodiments, the ZorC polypeptide comprises an amino acid sequence having at least 80% identity to a sequence selected from the group consisting of the polypeptides referenced in Table 8, rows 2-1174 columns R and S

In some embodiments, the ZorC polypeptide comprises an amino acid sequence having at least 80%, at least 85%, at least 90%, or at least 95% homology to a sequence selected from the group consisting of the polypeptides referenced in Table 8, rows 2-1174 columns R and S. In some embodiments, the ZorC polypeptide comprises the amino acid sequence having at least 80%, at least 85%, at least 95% homology within similar domain regions within a sequence selected from the group consisting of the polypeptides referenced in Table 8, rows 2-1174 columns R and S. In some embodiments, a ZorC homolog comprises an EH signature domain. In some embodiments, the ZorC polypeptide comprises an amino acid sequence having at least 80%, at least 85%, at least 90%, or at least 95% identity to a sequence selected from the group consisting of the polypeptides referenced in Table 8, rows 2-1174 columns R and S. In some embodiments, the ZorC polypeptide comprises the amino acid sequence having at least 80%, at least 85%, at least 95% identity within similar domain regions within a sequence selected from the group consisting of the polypeptides referenced in Table 8, rows 2-1174 column N. In some embodiments, the ZorC polypeptide comprises an amino acid sequence selected from the group consisting of SEQ ID NOs: the polypeptides referenced in Table 8, rows 2-1174 columns R and S.

In some embodiments, the ZorC polypeptide comprises an amino acid sequence encoded from a nucleic acid sequence having at least 80%, at least 85%, at least 90%, at least 95% homology to a sequence selected from the group consisting of the polynucleotides referenced in Table 8, rows 2-1174 columns R and T. In some embodiments, the ZorC polypeptide comprises the amino acid sequence encoded by a nucleic acid sequence having at least 80%, at least 85%, at least 95% homology within similar domain regions within a sequence selected from the group consisting of the polynucleotides referenced in Table 8, rows 2-1174 columns R and T. In some embodiments, the ZorC polypeptide comprises an amino acid sequence encoded from a nucleic acid sequence having at least 80%, at least 85%, at least 90%, at least 95% identity to a sequence selected from the group consisting of the polynucleotides referenced in Table 8, rows 2-1174 columns R and T. In some embodiments, the ZorC polypeptide comprises the amino acid sequence encoded by a nucleic acid sequence having at least 80%, at least 85%, at least 95% identity within similar domain regions within a sequence selected from the group consisting of the polynucleotides referenced in Table 8, rows 2-1174 columns R and T. In some embodiments, the ZorC polypeptide comprises an amino acid sequence encoded from a nucleic acid sequence selected from the group consisting of the polynucleotides referenced in Table 8, rows 2-1174 columns R and T.

In some embodiments, the zorC gene comprises a nucleic acid sequence having at least 80%, at least 85%, at least 90%, at least 95% homology to a sequence selected from the group consisting of the polynucleotides referenced in Table 8, rows 2-1174 columns R and T. In some embodiments, the zorC gene comprises a nucleic acid sequence having at least 80%, at least 85%, at least 90%, at least 95% identity to a sequence selected from the group consisting of the polynucleotides referenced in Table 8, rows 2-1174 columns R and T. In some embodiments, the nucleic acid sequence of a zorA gene comprises a nucleic acid sequence having at least 80%, at least 85%, at least 90%, or at least 95% homology to a sequence selected from the group consisting of the polynucleotides referenced in Table 8, rows 2-1174 columns R and T. In some embodiments, the nucleic acid sequence of a zorA gene comprises a nucleic acid sequence having at least 80%, at least 85%, at least 90%, or at least 95% identity to a sequence selected from the group consisting of the polynucleotides referenced in Table 8, rows 2-1174 columns R and T. In some embodiments, the nucleic acid sequence of a zorC gene comprises a nucleic acid sequence having at least 80%, at least 85%, at least 90%, or at least 95% homology within the nucleotide sequence encoding similar domain regions as a sequence selected from the group consisting of the polynucleotides referenced in Table 8, rows 2-1174 columns R and T. In some embodiments, the nucleic acid sequence of a zorC gene comprises a nucleic acid sequence having at least 80%, at least 85%, at least 90%, or at least 95% identity within the nucleotide sequence encoding similar domain regions as a sequence selected from the group consisting of the polynucleotides referenced in Table 8, rows 2-1174 columns R and T. In some embodiments, the zorC gene comprises a nucleic acid sequence selected from the group consisting of the polynucleotides referenced in Table 8, rows 2-1174 columns R and T.

As used herein, the term “ZorD” refers to the polynucleotide or expression product e.g., polypeptide encoded by the zorD gene. In some embodiments, the term “ZorD” refers to a ZorD polypeptide. In some embodiments, the product of the zorD gene comprises a pfam00176 domain. In some embodiments, the product of the zorD gene comprises a pfam00271 domain. In some embodiments, the product of the zorD gene contains a pfam00176 domain and a pfam00271 domain. In some embodiments, the product of the zorD gene comprises, a COG0553 domain. In some embodiments, the product of the zorD gene further contains a pfam04471 domain.

In some embodiments, a ZorD polypeptide comprises a pfam00176 domain. In some embodiments, a ZorD polypeptide comprises a pfam00271 domain. In some embodiments, a ZorD polypeptide comprises a pfam00176 domain and a pfam00271 domain. In some embodiments, a ZorD polypeptide comprises, a COG0553 domain. In some embodiments, a ZorD polypeptide comprises a pfam04471 domain. In some embodiments, a ZorD polypeptide comprises, a pfam00176 domain, a pfam00271 domain, a COG0553 domain, or a pfam04471 domain or any combination thereof. In some embodiments, a ZorD polypeptide comprises, a pfam00176 domain, a pfam00271 domain, a COG0553 domain, and a pfam04471 domain.

In some embodiments, a ZorD polypeptide is encoded by a gene positioned within 5-60 genes of a gene encoding ZorA, ZorB and/or ZorC in a genome of a prokaryotic cell. In some embodiments, a ZorD polypeptide is encoded by a gene positioned within 5 genes upstream (5′) or downstream (3′) to a gene encoding ZorA, ZorB and/or ZorC in a genome of a prokaryotic cell. In some embodiments, ZorD polypeptide is encoded by a gene positioned within 5 genes upstream or downstream to a gene encoding ZorA, ZorB and ZorC in a genome of a prokaryotic cell.

In some embodiments, a ZorD polypeptide is about 1200 amino acids long (median gene size).

In some embodiments, the ZorD polypeptide comprises an amino acid sequence having at least 80% homology to a sequence selected from the group consisting of the polypeptides referenced in Table 8, rows 2-1174 columns V and W. In some embodiments, the ZorD polypeptide comprises the amino acid sequence having at least 80% homology within similar domain regions within a sequence selected from the group consisting of the polypeptides referenced in Table 8, rows 2-1174 columns V and W. In some embodiments, a ZorD polypeptide homologue comprises a SW12/SNF2 helicase. In some embodiments, a ZorD polypeptide homologue comprises a Mrr-like nuclease domain. In some embodiments, the ZorD polypeptide comprises an amino acid sequence having at least 80% identity to a sequence selected from the group consisting of the polypeptides referenced in Table 8, rows 2-1174 columns V and W. In some embodiments, the ZorD polypeptide comprises the amino acid sequence having at least 80% identity within similar domain regions within a sequence selected from the group consisting of the polypeptides referenced in Table 8, rows 2-1174 columns V and W.

In some embodiments, the ZorD polypeptide comprises an amino acid sequence having at least 80%, at least 85%, at least 90%, at least 95% homology to a sequence selected from the group consisting of the polypeptides referenced in Table 8, rows 2-1174 columns V and W. In some embodiments, the ZorD polypeptide comprises the amino acid sequence having at least 80%, 85%, 90%, 95% homology within similar domain regions within a sequence selected from the group consisting of the polypeptides referenced in Table 8, rows 2-1174 columns V and W.

In some embodiments, the ZorD polypeptide comprises an amino acid sequence having at least 80%, at least 85%, at least 90%, at least 95% identity to a sequence selected from the group consisting of the polypeptides referenced in Table 8, rows 2-1174 columns V and W. In some embodiments, the ZorD polypeptide comprises the amino acid sequence having at least 80%, 85%, 90%, 95% identity within similar domain regions within a sequence selected from the group consisting of the polypeptides referenced in Table 8, rows 2-1174 columns V and W. In some embodiments, the ZorD polypeptide comprises an amino acid sequence selected from the group consisting of the polypeptides referenced in Table 8, rows 2-1174 columns V and W.

In some embodiments, the ZorD polypeptide comprises an amino acid sequence encoded from a nucleic acid sequence having at least 80%, at least 85%, at least 90%, or at least 95% homology to a sequence selected from the group consisting of the polynucleotides referenced in Table 8, rows 2-1174 columns V and X. In some embodiments, the ZorD polypeptide comprises the amino acid sequence encoded by a nucleic acid sequence having at least 80%, 85%, 90%, 95% homology within similar domain regions within a sequence selected from the group consisting of the polynucleotides referenced in Table 8, rows 2-1174 columns V and X. In some embodiments, the ZorD polypeptide comprises an amino acid sequence encoded from a nucleic acid sequence having at least 80%, at least 85%, at least 90%, or at least 95% identity to a sequence selected from the group consisting of the polynucleotides referenced in Table 8, rows 2-1174 columns V and X. In some embodiments, the ZorD polypeptide comprises the amino acid sequence encoded by a nucleic acid sequence having at least 80%, 85%, 90%, 95% identity within similar domain regions within a sequence selected from the group consisting of the polynucleotides referenced in Table 8, rows 2-1174 columns V and X. In some embodiments, the ZorD polypeptide comprises an amino acid sequence encoded from a nucleic acid sequence selected from the group consisting of the polynucleotides referenced in Table 8, rows 2-1174 columns V and X.

In some embodiments, the zorD gene comprises a nucleic acid sequence having at least 80%, at least 85%, at least 90%, or at least 95% homology to a sequence selected from the group consisting of the polynucleotides referenced in Table 8, rows 2-1174 columns V and X. In some embodiments, the zorD gene comprises a nucleic acid sequence having at least 80%, at least 85%, at least 90%, or at least 95% identity to a sequence selected from the group consisting of the polynucleotides referenced in Table 8, rows 2-1174 columns V and X. In some embodiments, the nucleic acid sequence of a zorD gene comprises a nucleic acid sequence having at least 80%, at least 85%, at least 90%, or at least 95% homology within the nucleotide sequence encoding similar domain regions as a sequence selected from the group consisting of the polynucleotides referenced in Table 8, rows 2-1174 columns V and X. In some embodiments, the nucleic acid sequence of a zorD gene comprises a nucleic acid sequence having at least 80%, at least 85%, at least 90%, or at least 95% identity within the nucleotide sequence encoding similar domain regions as a sequence selected from the group consisting of the polypeptides referenced in Table 8, rows 2-1174 column P. In some embodiments, the nucleic acid sequence of the zorD gene is selected from the group consisting of the polynucleotides referenced in Table 8, rows 2-1174 columns V and X.

As used herein, the term “ZorE” refers to the polynucleotide or expression product e.g., polypeptide encoded by the zorE gene. In some embodiments, the term “ZorE” refers to a ZorE polypeptide. In some embodiments, the product of the zorE gene comprises a pfam01844 domain. In some embodiments, the product of the zorE gene comprises a COG3183 domain. In some embodiments, the product of the zorE gene contains a pfam01844 domain and a COG3183 domain. In some embodiments, the product of the zorE gene comprises an HNH endonuclease.

In some embodiments, a ZorE polypeptide comprises a pfam01844 domain. In some embodiments, a ZorE polypeptide comprises a COG3183 domain. In some embodiments, a ZorE polypeptide comprises a pfam01844 domain and a COG31383 domain. In some embodiments, a ZorE polypeptide comprises a HNH endonuclease.

In some embodiments, a ZorE polypeptide is encoded by a gene positioned within 5-60 genes of a gene encoding ZorA and/or ZorB in a genome of a prokaryotic cell. In some embodiments, a ZorE polypeptide is encoded by a gene positioned within 5 genes upstream (5′) or downstream (3′) to a gene encoding ZorA and/or ZorB in a genome of a prokaryotic cell. In some embodiments, ZorE polypeptide is encoded by a gene positioned within 5 genes upstream and downstream to a gene encoding ZorA and/or ZorB in a genome of a prokaryotic cell.

In some embodiments, a ZorE polypeptide is about 367 amino acids long (median gene size).

In some embodiments, the ZorE polypeptide comprises an amino acid sequence having at least 80% homology to a sequence selected from the group consisting of the polypeptides referenced in Table 8, rows 1175-1830 columns Z and AA. In some embodiments, the ZorE polypeptide comprises the amino acid sequence having at least 80% homology within similar domain regions within a sequence selected from the group consisting of the polypeptides referenced in Table 8, rows 1175-1830 columns Z and AA. In some embodiments, a ZorE polypeptide homologue comprises a HNH endonuclease. In some embodiments, the ZorE polypeptide comprises an amino acid sequence having at least 80% identity to a sequence selected from the group consisting of the polypeptides referenced in Table 8, rows 1175-1830 columns Z and AA. In some embodiments, the ZorE polypeptide comprises the amino acid sequence having at least 80% identity within similar domain regions within a sequence selected from the group consisting of the polypeptides referenced in Table 8, rows 1175-1830 columns Z and AA.

In some embodiments, the ZorE polypeptide comprises an amino acid sequence having at least 80%, at least 85%, at least 90%, or at least 95% homology to a sequence selected from the group consisting of the polypeptides referenced in Table 8, rows 1175-1830 columns Z and AA. In some embodiments, the ZorE polypeptide comprises the amino acid sequence having at least 80%, 85%, 90%, 95% homology within similar domain regions within a sequence selected from the group consisting of the polypeptides referenced in Table 8, rows 1175-1830 columns Z and AA. In some embodiments, the ZorE polypeptide comprises an amino acid sequence having at least 80%, at least 85%, at least 90%, or at least 95% identity to a sequence selected from the group consisting of the polypeptides referenced in Table 8, rows 1175-1830 columns Z and AA. In some embodiments, the ZorE polypeptide comprises the amino acid sequence having at least 80%, 85%, 90%, 95% identity within similar domain regions within a sequence selected from the group consisting of the polypeptides referenced in Table 8, rows 1175-1830 columns Z and AA. In some embodiments, the ZorE polypeptide comprises an amino acid sequence selected from the group consisting of the polypeptides referenced in Table 8, rows 1175-1830 columns Z and AA.

In some embodiments, the ZorE polypeptide comprises an amino acid sequence encoded from a nucleic acid sequence having at least 80%, at least 85%, at least 90%, or at least 95% homology to a sequence selected from the group consisting of the polynucleotides referenced in Table 8, rows 1175-1830 columns Z and AB. In some embodiments, the ZorE polypeptide comprises the amino acid sequence encoded from a nucleic acid sequence having at least 80%, 85%, 90%, 95% homology within similar domain regions within a sequence selected from the group consisting of the polynucleotides referenced in Table 8, rows 1175-1830 columns Z and AB. In some embodiments, the ZorE polypeptide comprises an amino acid sequence encoded from a nucleic acid sequence having at least 80%, at least 85%, at least 90%, or at least 95% identity to a sequence selected from the group consisting of the polynucleotides referenced in Table 8, rows 1175-1830 columns Z and AB. In some embodiments, the ZorE polypeptide comprises the amino acid sequence encoded from a nucleic acid sequence having at least 80%, 85%, 90%, 95% identity within similar domain regions within a sequence selected from the group consisting of the polynucleotides referenced in Table 8, rows 1175-1830 columns Z and AB. In some embodiments, the ZorE polypeptide comprises an amino acid sequence encoded from a nucleic acid sequence selected from the group consisting of the polynucleotides referenced in Table 8, rows 1175-1830 columns Z and AB.

In some embodiments, the zorE gene comprises a nucleic acid sequence having at least 80%, at least 85%, at least 90%, or at least 95% homology to a sequence selected from the group consisting of the polynucleotides referenced in Table 8, rows 1175-1830 columns Z and AB. In some embodiments, the zorE gene comprises a nucleic acid sequence having at least 80%, at least 85%, at least 90%, or at least 95% identity to a sequence selected from the group consisting of the polynucleotides referenced in Table 8, rows 1175-1830 columns Z and AB. In some embodiments, the nucleic acid sequence of a zorE gene comprises a nucleic acid sequence having at least 80%, at least 85%, at least 90%, or at least 95% homology within the nucleotide sequence encoding similar domain regions as a sequence selected from the group consisting of the polynucleotides referenced in Table 8, rows 1175-1830 columns Z and AB. In some embodiments, the nucleic acid sequence of a zorE gene comprises a nucleic acid sequence having at least 80%, at least 85%, at least 90%, or at least 95% identity within the nucleotide sequence encoding similar domain regions as a sequence selected from the group consisting of the polynucleotides referenced in Table 8, rows 1175-1830 columns Z and AB. In some embodiments, the nucleic acid sequence of the zorE gene is selected from the group consisting of the polynucleotides referenced in Table 8, rows 1175-1830 columns Z and AB.

A skilled artisan would appreciate that a “functional portion of a Defense System I component” or “functional fragment of Defense System I component” or “functional portion of a ZORYA defense system component” or “functional fragment of ZORYA defense system component” refers to a functional portion of a ZORYA polynucleotide or polypeptide, as disclosed herein, which expression is sufficient to elicit an anti-phage activity alone or in combination with the at least one of the other ZORYA polynucleotides or polypeptides disclosed herein or functional portions thereof.

As used herein, the terms “ZorA”, “ZorB”, “ZorC”, “ZorD”, “ZorE”, “zorA”, “zorB”, “zorC”, “zorD”, and “zorE”, also refer to functional ZorA, ZorB, ZorC, ZorD, ZorE, zorA, zorB, zorC, zorD, and zorE, homologs, which exhibit the desired activity (i.e., conferring phage resistance). Such homologs can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptides referenced in Table 8, rows 2-1830 columns J and K; rows 2-1830 columns N and O; rows 2-1174 columns R and S; rows 2-1174 columns V and W; and rows 1175-1830 columns Z and AA, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide referenced in Table 8, rows 2-1830 columns J and L; rows 2-1830 columns N and P; rows 2-1174 columns R and T; rows 2-1174 columns V and X; and rows 1175-1830 column Z and AB, respectively. In some embodiments, such homologs comprise at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% homology within similar domain regions of the polypeptide referenced in Table 8, rows 2-1830 columns J and K; rows 2-1830 columns N and O; rows 2-1174 columns R and S; rows 2-1174 column V and W; and rows 1175-1830 column Z and AA, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity within nucleotide sequences encoding similar domain regions to the polynucleotide referenced in Table 8, rows 2-1830 columns J and L; rows 2-1830 columns N and P; rows 2-1174 columns R and S; rows 2-1174 columns V and X; and rows 1175-1830 columns Z and AB, respectively.

Table 8 presents embodiments of components of Defense System Ia and Defense System Ib that may be found in a diverse array of bacteria and archaea genomes (referenced in Table 18). Table 8 presents embodiments of nucleic acid sequences encoding gene cassettes of Defense System Ia and Defense System Ib (referenced in Table 18).

The Thoeris Defense System

In some embodiments, a defense system disclosed herein comprises a Thoeris anti-phage defense system. (Table 9)

In some embodiments, a Thoeris defense system provides a host cell with resistance to entry of foreign nucleic acid invasion. In some embodiments, a host cell expressing a functional Thoeris defense system (Defense System II) provides the host cell resistance foreign nucleic acid invasion.

In some embodiments, a Thoeris defense system provides a host cell with resistance to at least one phage. In some embodiments, expression of a Thoeris defense system in bacteria protects the bacteria from phage infection. In some embodiments, expression of a Thoeris defense system in bacteria protects the bacteria from plasmid transformation. In some embodiments, expression of a Thoeris defense system in bacteria protects the bacteria from phage infection and from plasmid transformation.

In some embodiments, a Thoeris defense system provides a host cell with resistance to phage infection. In some embodiments, a Thoeris defense system provides a host cell with resistance to plasmid transformation.

In some embodiments, expression of a Thoeris defense system in bacteria protects the bacteria from phage infection.

As used herein, the term “a Thoeris anti-phage defense system” may be used interchangeably with the term “a Defense System II”, having all the same meanings and qualities. A skilled artisan would appreciate that the term “Thoeris system” may be used interchangeably in some embodiments with “Thoeris defense system”, “Thoeris the defense system”, “Thoeris anti-phage system”, and “Defense System II”, having all the same meanings and qualities.

In some embodiments, a Thoeris defense system (Defense System II) provides a host cell with resistance to plasmid transformation. In some embodiments, a host cell expressing a functional Thoeris defense system (Defense System II) provides a host cells with resistance to plasmid transformation.

In some embodiments, a Thoeris defense system (Defense system II) provides a host cell with resistance to entry of conjugative elements. In some embodiments, a host cell expressing a function Thoeris defense system (Defense System II) provides the host cell resistance from entry of conjugative elements.

In some embodiments, a microbial species does not comprise an endogenous Defense System II. In some embodiments, a microbial species does not express an endogenous Defense System II. In some embodiments, a microbial species does not express an endogenous functional Defense System II.

In some embodiments, a bacterial species does not comprise an endogenous Defense System II. In some embodiments, a bacterial species does not express an endogenous Defense System II. In some embodiments, a bacterial species does not express an endogenous functional Defense System II.

A Thoeris defense system comprises, in some embodiments, a nucleic acid construct comprising a nucleic acid sequence encoding a ThsA polypeptide comprising a pfam13289 domain, and a ThsB polypeptide comprising a pfam13676 domain or a pfam08937 domain or a pfam08357 domain or a pfam13289, or a pfam14519 or a pfam08357 domain or any combination thereof. In some embodiments, a Thoeris defense system comprises a nucleic acid construct comprising a nucleic acid sequence comprising a thsA gene and a thsB gene. In some embodiments, a Thoeris defense system comprises a nucleic acid construct comprising a nucleic acid sequence comprising a thsA gene and multiple copies of a thsB gene. In some embodiments, a Thoeris defense system comprises 2 copies of a thsB gene. In some embodiments, a Thoeris defense system comprises 3 copies of a thsB gene. In some embodiments, a Thoeris defense system comprises 4 copies of a thsB gene. In some embodiments, a Thoeris defense system comprises 5 copies of a thsB gene. In some embodiments, a Thoeris defense system comprises 6 copies of a thsB gene. In some embodiments, a Thoeris defense system comprises 7 copies of a thsB gene. In some embodiments, a Thoeris defense system comprises 8 copies of a thsB gene. In some embodiments, a Thoeris defense system comprises 9 copies of a thsB gene. In some embodiments, a Thoeris defense system comprises 9 copies of a thsB gene. In some embodiments, a Thoeris defense system comprises 1-10 copies of a thsB gene.

In some embodiments, each copy of a thsB gene comprises the same nucleotide sequence. In some embodiments, each copy of a thsB gene comprises a different nucleotide sequence. In some embodiments, each copy of a thsB gene encodes a ThsB polypeptide having the same amino acid sequence. In some embodiments, each copy of a thsB gene encodes a ThsB polypeptide having a different amino acid sequence. In some embodiments, the source of the different nucleotide sequences comprises different donor species. In some embodiments, the source of the difference amino acid sequences comprises different donor species

A Thoeris defense system (Defense System II) comprises, in some embodiments, a nucleic acid construct comprising a nucleic acid sequence encoding a ThsA polypeptide comprising a pfam13289 or a pfam14519 domain or a combination thereof, and a ThsB polypeptide comprising a pfam08937 domain or a pfam13676 domain or a pfam08357 domain or any combination thereof. In some embodiments, a Thoeris defense system comprises a nucleic acid construct comprising a nucleic acid sequence encoding a ThsA polypeptide and a ThsB polypeptide. In some embodiments, a Thoeris defense system comprises a nucleic acid construct comprising a nucleic acid sequence encoding a ThsA polypeptide and 1-10 ThsB polypeptides.

In some embodiments, a Thoeris defense system (Defense System II) comprises a ThsA polypeptide comprising a pfam13289 or a pfam14519 domain or a combination thereof, and a ThsB polypeptide comprising a pfam13676 domain or a pfam08937 domain or a pfam08357 domain or a pfam13289 or a pfam14519 or a pfam08357 domain or any combination thereof. In some embodiments, a Thoeris defense system (Defense System II) comprises a ThsA polypeptide comprising a pfam13289 or a pfam14519 domain or a combination thereof, and 1-10 ThsB polypeptides comprising a pfam13676 domain or a pfam08937 domain or a pfam08357 domain or a pfam13289 or a pfam14519 or a pfam08357 domain or a combination thereof. In some embodiments, a Thoeris defense system (Defense System II) having an anti-phage activity comprises a ThsA polypeptide comprising a pfam13289 or a pfam14519 domain or a combination thereof, and a ThsB polypeptide comprising a pfam13676 domain or a pfam08937 domain or a pfam08357 domain or a pfam13289 or a pfam14519 or a pfam08357 domain or any combination thereof. In some embodiments, a Thoeris defense system (Defense System II) having an anti-phage activity comprises a ThsA polypeptide comprising a pfam13289 or a pfam14519 domain or a combination thereof, and 1-10 ThsB polypeptides comprising a pfam13676 domain or a pfam08937 domain or a pfam08357 domain or a pfam13289, or a pfam14519 or a pfam08357 domain or any combination thereof. In some embodiments, a Thoeris defense system (Defense System II) having an anti-phage activity comprises a ThsA polypeptide comprising a pfam13289 or a pfam14519 domain or a combination thereof, and 1-5 ThsB polypeptides comprising a pfam13676 domain or a pfam08937 domain or a pfam08357 domain or a pfam13289 or a pfam14519 or a pfam08357 domain or any combination thereof. In some embodiments, a Thoeris defense system (Defense System II) having an anti-phage activity comprises a ThsA polypeptide comprising a pfam13289 or a pfam14519 domain or a combination thereof, and 1, 2, 3, 4, 5, 6, 7, 8, 9, or 10 ThsB polypeptides comprising a pfam13676 domain or a pfam08937 domain or a pfam08357 domain or a pfam13289 or a pfam14519 or a pfam08357 domain or any combination thereof.

In some embodiments, a Thoeris defense system (Defense System II) comprises at least two different polypeptide components selected from a ThsA polypeptide comprising a pfam13289 or a pfam14519 domain or a combination thereof, and a ThsB polypeptide comprising a pfam13676 domain or a pfam08937 domain or a pfam08357 domain or a pfam13289 or a pfam14519 or a pfam08357 domain or any combination thereof. In some embodiments, a Thoeris defense system (Defense System II) comprises at least two different polypeptide components selected from a ThsA polypeptide comprising a pfam13289 or a pfam14519 domain or a combination thereof, and 1-10 ThsB polypeptides comprising a pfam13676 domain or a pfam08937 domain or a pfam08357 domain or a pfam13289 or a pfam14519 or a pfam08357 domain or a combination thereof. In some embodiments, a Thoeris defense system (Defense System II) having an anti-phage activity comprises at least two different polypeptide components selected from a ThsA polypeptide comprising a pfam13289 or a pfam14519 domain or a combination thereof, and a ThsB polypeptide comprising a pfam13676 domain or a pfam08937 domain or a pfam08357 domain or a pfam13289 or a pfam14519 or a pfam08357 domain or any combination thereof. In some embodiments, a Thoeris defense system (Defense System II) having an anti-phage activity comprises at least two different polypeptide components selected from a ThsA polypeptide comprising a pfam13289 or a pfam14519 domain or a combination thereof, and 1-10 ThsB polypeptides comprising a pfam13676 domain or a pfam08937 domain or a pfam08357 domain or a pfam13289, or a pfam14519 or a pfam08357 domain or any combination thereof. In some embodiments, a Thoeris defense system (Defense System II) having an anti-phage activity comprises at least two different polypeptide components selected from a ThsA polypeptide comprising a pfam13289 or a pfam14519 domain or a combination thereof, and 1-5 ThsB polypeptides comprising a pfam13676 domain or a pfam08937 domain or a pfam08357 domain or a pfam13289 or a pfam14519 or a pfam08357 domain or any combination thereof. In some embodiments, a Thoeris defense system (Defense System II) having an anti-phage activity comprises at least two different polypeptide components selected from a ThsA polypeptide comprising a pfam13289 or a pfam14519 domain or a combination thereof, and 1, 2, 3, 4, 5, 6, 7, 8, 9, or 10 ThsB polypeptides comprising a pfam13676 domain or a pfam08937 domain or a pfam08357 domain or a pfam13289 or a pfam14519 or a pfam08357 domain or any combination thereof.

In some embodiments, a Defense System II comprises a ThsA polypeptide comprising a pfam13289 domain or a pfam14519 domain or a combination thereof, or comprises an amino acid sequence having at least 80% identity to a sequence selected from the group referenced in Table 9 rows 2-2100 columns I and J; or a ThsB polypeptide comprising a pfam13676 domain or a pfam08937 domain or a pfam08357 domain or a combination thereof, or comprises an amino acid sequence having at least 80% identity to a sequence selected from the group referenced in Table 9 rows 2-2100 columns M and N, Q and R, U and V, Y and Z, and AC and AD, or a combination thereof; or a combination thereof.

In some embodiments, the amino acid sequences of more than one copy of a Thsb polypeptide present in a Defense System II are identical. In some embodiments, the amino acid sequences of more than one copy of a Thsb polypeptide present in a Defense System II are homologous. In some embodiments, the amino acid sequences of more than one copy of a Thsb polypeptide present in a Defense System II are not identical. In some embodiments, the amino acid sequences of more than one copy of a Thsb polypeptide present in a Defense System II comprise the same functional activity.

In some embodiments, a Thoeris defense system having an anti-phage activity comprises a nucleic acid construct comprising a nucleic acid sequence encoding a ThsA polypeptide and a ThsB polypeptide. In some embodiments, the Thoeris system having an anti-phage activity comprises a nucleic acid construct comprising a nucleic acid sequence encoding a ThsA polypeptide and encoding 1-10 copies of a ThsB polypeptide. In some embodiments, a Thoeris defense system having an anti-phage activity comprises a nucleic acid construct comprising a nucleic acid sequence comprising a thsA gene and a thsB gene. In some embodiments, a Thoeris defense system having an anti-phage activity comprises a nucleic acid construct comprising a nucleic acid sequence comprising a thsA gene and 1-10 copies of a thsB gene.

In some embodiments, the Thoeris anti-phage defense system comprises a nucleic acid construct comprising nucleic acid sequences encoding a cassette comprising thsA and thsB genes. In some embodiments, the Thoeris anti-phage defense system comprises a nucleic acid construct comprising nucleic acid sequences encoding a cassette comprising thsA and 1-10 copies of a thsB genes. In some embodiments, the Thoeris anti-phage defense system comprises a nucleic acid construct comprising nucleic acid sequences encoding a cassette comprising thsA and 1, 2, 3, 4, 5, 6, 7, 8, 9, or 10 copies of a thsB genes.

In some embodiments, a Thoeris (Defense System II) gene cassette comprises the nucleic acid sequence:

(SEQ ID NO: 10; construct 31 Table 4)

| | |
|---|---|
| 1 | atgtttttag aaatttataa ttaatttact aatatattgc gataattaga accatttgaa |
| 61 | ttacagaagt gccaccaacg aggaagcaat caattaaata gtaagtttaa taattttatt |
| 121 | aagaaattga tttgttatac gaaattttat tatatgataa aatatttatg taataaatgt |
| 181 | aaatttgtat attaggtaag gggaatgaaa atgaatccaa tagttgaact atttattaaa |
| 241 | gattttacaa aagaagttat ggaagagaat gctgcaattt ttgcaggagc gggtttatca |
| 301 | atgtctgttg ggtatgttag ttgggcgaag ttattggagc ctattgctca agaaattgga |
| 361 | ttagatgtaa ataaagaaaa tgatttagta agcttggcgc aatattattg taatgagaat |
| 421 | cagggtaata gaggtagaat taatcaaatt atcctagatg aattttctcg aaaagtagat |
| 481 | ttaactgaaa atcacaaaat tttagctcga ttaccaattc atacttattg gacgacaaat |
| 541 | tatgataggt taattgaaaa ggcattagaa gaagaaaata agattgctga tgtaaagtat |
| 601 | acggtaaaac aattagctac tacaaaggta aaaagagatg cagttgtata taaaatgcat |
| 661 | ggagacgtgg aacatccctc tgaggcagtt ttgattaaag atgattatga aaagtattct |
| 721 | ataaaaatgg atccgtatat taaggcgttg agtggggatc tagtttctaa gacattttta |
| 781 | tttgttggat ttagttttac tgatcccaat ctagattata tattaagtcg agtaaggagt |
| 841 | gcttatgaaa gggatcaaag aagacactat tgtttaataa aaaaagaaga gcgaagacca |
| 901 | gatgaattag aggcggattt tgagtataga gtaagaaaac aggaattatt tattagtgat |
| 961 | ttatcacgtt ttaatataaa gactattgta cttaataatt acaatgagat taccgagata |
| 1021 | ttacaacgaa ttgaaaataa tataaaaact aaaacagtat ttttatctgg tagtgcggtt |
| 1081 | gaatataatc attgggagac agaacatgct gaacaattca ttcatcagtt aagtaaagaa |
| 1141 | ttaattagaa aggattttaa tatagtatct ggttttggac ttggtgtagg gagttttgtt |
| 1201 | atcaatggag ttctagaaga attgtatatg aaccagggaa ctatagatga tgatagactt |
| 1261 | attctaagac cgtttccaca ggggaaaaag ggcgaagagc aatgggataa atatagacga |
| 1321 | gatatgatta caagaactgg ggtaagcatc tttttatatg gaaataaaat agacaaggga |
| 1381 | caggttgtta aggctaaggg tgtccaatct gaatttaata tttcttttga acaaaataat |
| 1441 | tacgttgttc cggtaggtgc aactggttat attgcaaaag atttatggaa taaagtaaat |
| 1501 | gaggaatttg aaacgtatta cccaggtgct gatgcaagga tgaaaaaatt gttcggagaa |
| 1561 | ttgaataatg aagcattatc aatagaggaa ctgattaaca ctataataga gtttgtagaa |
| 1621 | atcctatcca actaatcatt actgattatt aatcagagag gaactgttaa attatggcga |
| 1681 | aaagagtttt ttttagtttc cattatcaag atgtaattga ttttagagta aatgttgtaa |
| 1741 | gaaatcattg ggtaacaaaa ttaaatcaaa gtgctgcggg ggtctttgat gcatctcttt |
| 1801 | gggaagatgc aaaaaaaaca agtgatatag cacttaaaag attaataaat ggcgggctaa |
| 1861 | acaatacctc agttacatgt gttttaattg gatctcagac ttttaataga agatgggtga |
| 1921 | gatatgaaat tatgaaaagt atagaaaaag gaaataagat aataggaatt catattaatg |
| 1981 | cttttaaaga taaatatgga aatattaaaa gtaaagggcc taatcctttc gattatttag |
| 2041 | ggtatcaata tagttcagac ggcaaacagt tacatttgta tgagtggaca gggggaaaat |
| 2101 | gggaggaata taaggatttg gcaccatata gggtgaatca aattgctcca gaatcactta |
| 2161 | gaggaaaatt ctatagtttg tcatcggttt atcgtgtgta tgattgggtt gctgatgatg |
| 2221 | gatataacaa attttcctct tgggtgaact aattaagttt tggataaaat tataaataga |
| 2281 | attgctattg ggattagata caaaatgatt aaataagaaa agtacctata aaagtaggtg |
| 2341 | cttttttctt tgttatatag aaattacaca ttaatttata tttaggttat tttaaaaaga |
| 2401 | aaaagtgatg aatgatggta ttcttacaat taatagttac ttataactat atgtacatga |
| 2461 | aaaacatcta tatatgc. |

The coding regions for each of the thsA and thsB gene sequences within this embodiment of a Thoeris cassette (SEQ ID NO: 10) are as follows: nucleotides 205-1635 encode a ThsA polypeptide; and nucleotides 1674-2252 encode a ThsB polypeptide.

In some embodiments, a Defense System II comprising a ThsA polypeptide and a ThsB polypeptide is encoded by the nucleic acid sequence set forth in SEQ ID NO: 10. In some embodiments, a Defense System II comprising a ThsA polypeptide and a ThsB polypeptide, is encoded by a nucleic acid sequence having at least 80% homology to the sequence set forth in SEQ ID NO: 10. In some embodiments, a Defense System II comprising a ThsA polypeptide and a ThsB polypeptide, is encoded by a nucleic acid sequence having at least 85% homology, or 90% homology, or 95% homology, or 97% homology, or 98% homology, or 99% homology to the sequence set forth in SEQ ID NO: 10. In some embodiments, a Defense System II comprising a ThsA polypeptide and a ThsB polypeptide, is encoded by a nucleic acid sequence having at least 80% identity to the sequence set forth in SEQ ID NO: 10. In some embodiments, a Defense System II comprising a ThsA polypeptide and a ThsB polypeptide, is encoded by a nucleic acid sequence having at least 85% identity, or 90% identity, or 95% identity, or 97% identity, or 98% identity, or 99% identity to the sequence set forth in SEQ ID NO: 10.

In some embodiments, a Thoeris (Defense System II) gene cassette comprises the nucleic acid sequence:

(SEQ ID NO: 11; construct 32 Table 4)

| | |
|---|---|
| 1 | gttaagcttt aggcggacat cagcaacgat gtccgctttt atcatcttaa cagcaataca |
| 61 | aggaggttta cgtggcggat gaaactctga aacagtatat gggcctgtac aaatgttgtt |
| 121 | agcaatctca agaaagccca ggagtagata tgattcactg aacagatgtt caatttaaat |
| 181 | ctaataatga agagggccta gtcaaaggcg ctcttcatta ttagataaga cataatcatt |
| 241 | acacaagatt tatgtcatgg ttctatattc atattttcat tgtaataaat tgaaaatatg |
| 301 | aatataatag gtagtgatcg caatactaac attggaggtt ttatgtggaa gtttaaagaa |
| 361 | ttaataagag ataagacatt tagaaggtgg gctctgagta taattctgac tattccaact |
| 421 | agtgtgagta cattcatttc atttctagac ttggatgctc gttgtagatt aattatttta |
| 481 | cttatattag tagggttgtc gctagtcata ataattgttc aattcattag actacttttt |
| 541 | atgaataata tcactcttaa tctcaatggt tccgaagttg aaattaaaaa aggtgatatt |
| 601 | tttgaagtgc caagaaataa ttataaagtc attgcattta atgagtattt tgacactcaa |
| 661 | gtagatgacg taataatcgc ccgcgaaaca ttaaatggtc aatatataaa gcgttattat |
| 721 | tcacatcaag atatcactga gttggatcag aaaattaaag atgacgtaaa acttaaaatt |
| 781 | gaagaaaaaa atgttgaaag accttttgga ggaaaaacaa cacgttacag cttgggatca |
| 841 | gtctttaaag atatggattt ttttctagtt gctttttcga agtttgatag ggaaaatcgt |
| 901 | gcccaattaa aactaaatga atatgcaagc tgcatgctta atgtatggaa tgaaatcaat |
| 961 | acattacatg caagcaaaga agtgtttatc cccttattag gttctggaat aacaagacat |
| 1021 | gtggattctg atgttggagt caatgaacta ttacatataa tgttgtggac atttcagatc |
| 1081 | agtaaagtaa agtttagaga accagccaaa gttacaattc ttttgtacaa aaatgatcac |
| 1141 | aagaagatca atttttataa gttaaaggag tttgagaaga atgggttata ggaatggaaa |
| 1201 | ttatgctgct ttctatgtca gtgaaccgtt tagtgaaagt agtttagggg ctaatgcaac |
| 1261 | caaggatttt gtgtcgtata acatgttaag agcatggaaa ggaaaagata ataactaccc |
| 1321 | atttaatgac tcccacgata aaacatacaa tgtacgggac ggtagtgatt gggagaaaac |
| 1381 | attaaagcca agattaagaa aaagattaga tcaatcaaaa aatataatct tttttttaag |
| 1441 | taagcatact gaaaacagca aagctttgcg agaagaaatt gattacggta ttaatgtgaa |
| 1501 | ggggctacca gttatcgttg tataccctga actttccgaa aagagtgaca tcatcgactg |
| 1561 | cactacaaag gttttcagat cagaagtggt gaatttatgg agtcgagtcc ctgtttttaa |
| 1621 | agactctatg ctaaaggtac caactattca tattccttat aaaaaagacc aaattaaaaa |
| 1681 | agctttagag aacaaagatt ttatgattaa ttcaaagatt tctgcaggtt cggtttattt |
| 1741 | ttatccatgt taatctatta tgggtgaata taatttaaag agaaagaata gattttcagt |
| 1801 | ttcgtcctca tggtagctca ttaattcaaa aacctctatg accaatttaa tttattgagg |
| 1861 | aggtagaggt tttggtatta aaatgatgaa gatagattat tggatatgaa tttcatttta |
| 1921 | tctagtttac ataatatata ttctaggaag ttaaatcttt aatgaaaatt ctgtctcgtt |
| 1981 | atcttaagtg tacttagttg gtgagacaga attaaaaaaa taggatgtat tacgatttgt |
| 2041 | atttatttct. |

The coding regions for each of the thsA and thsB gene sequences within this embodiment of a Thoeris cassette (SEQ ID NO: 11) are as follows: nucleotides 298-1191 encode a ThsA polypeptide; and nucleotides 1181-1753 encode a ThsB polypeptide.

In some embodiments, a Defense System II comprising a ThsA polypeptide and a ThsB polypeptide is encoded by the nucleic acid sequence set forth in SEQ ID NO: 11. In some embodiments, a Defense System II comprising a ThsA polypeptide and a ThsB polypeptide, is encoded by a nucleic acid sequence having at least 80% homology to the sequence set forth in SEQ ID NO: 11. In some embodiments, a Defense System II comprising a ThsA polypeptide and a ThsB polypeptide, is encoded by a nucleic acid sequence having at least 85% homology, or 90% homology, or 95% homology, or 97% homology, or 98% homology, or 99% homology to the sequence set forth in SEQ ID NO: 11. In some embodiments, a Defense System II comprising a ThsA polypeptide and a ThsB polypeptide, is encoded by a nucleic acid sequence having at least 80% identity to the sequence set forth in SEQ ID NO: 11. In some embodiments, a Defense System II comprising a ThsA polypeptide and a ThsB polypeptide, is encoded by a nucleic acid sequence having at least 85% identity, or 90% identity, or 95% identity, or 97% identity, or 98% identity, or 99% identity to the sequence set forth in SEQ ID NO: 11.

In some embodiments, a construct comprising the Thoeris defense system encodes one component of the defense system, whereby multiple constructs may be used to assemble the functional defense system. In some embodiments, the components of a Thoeris defense system comprise genes thsA and thsB. In some embodiments, multiple thsB genes are present in the gene cassette of the defense system locus. In some embodiments, the components of a Thoeris defense system consist of genes thsA and thsB, wherein the copy number of the thsB gene is between 1 and 10. In some embodiments, the components of a Thoeris defense system comprise nucleic acid sequences encoding a ThsA polypeptide and a ThsB polypeptide. In some embodiments, the components of a Thoeris defense system consist of nucleic acid sequences encoding a ThsA polypeptide and a ThsB polypeptide. In some embodiments, the components of a Thoeris defense system consist of nucleic acid sequences encoding a ThsA polypeptide and 1-10 copies of a ThsB polypeptide. In some embodiments, the components of a Thoeris defense system consist of nucleic acid sequences encoding a ThsA polypeptide and 1, 2, 3, 4, 5, 6, 7, 8, 9, or 10 copies of a ThsB polypeptide.

In some embodiments, a construct comprising the Thoeris defense system encodes one component of the defense system, whereby a second construct may be used to assemble the functional defense system. In some embodiments, a construct comprising the Thoeris defense system encodes one component of the defense system, whereby additional constructs may be used to assemble the functional defense system. For example, in some embodiments each of ThsA and ThsB may be encoded by nucleic acid sequences comprised in different constructs, wherein expression of the combination of constructs produces a functional Thoeris defense system.

In some embodiments, the components making up a functional Thoeris anti-phage defense system comprise a ThsA polypeptide and a ThsB polypeptide, each encoded by a thsA and a thsB gene, respectively.

In some embodiments, a Thoeris defense system having an anti-phage activity comprise a nucleic acid encoding a ThsA polypeptide. In some embodiments, a Thoeris defense system having an anti-phage activity comprise a nucleic acid encoding a ThsA polypeptide and a ThsB polypeptide.

In some embodiments, a Thoeris defense system having an anti-phage activity comprise a nucleic acid comprising a thsA gene. In some embodiments, a Thoeris defense system having an anti-phage activity comprise a nucleic acid comprising a thsA gene and a thsB gene. In some embodiments, a Thoeris defense system having an anti-phage activity comprise a nucleic acid comprising a thsA gene and 1-10 thsB genes.

In some embodiments, a Thoeris defense system having an anti-phage activity comprises a nucleic acid construct comprising nucleic acids encoding polypeptides in a set order. In one embodiment, the 5′ to 3′ order of polypeptides encoded is ThsA and ThsB. In some embodiments the 5′ to 3′ order of polypeptides does not affect the anti-phage activity. In some embodiments the 5′ to 3′ order of polypeptides does affect the anti-phage activity.

In some embodiments, the 5′ to 3′ order of polypeptides is random, for example any order of ThsA and ThsB. In some embodiments, the 5′ to 3′ order of polypeptides is random, for example any order of ThsA and 1-10 copies of ThsB, wherein ThsB may be encoded both 5′ and 3′ of the gene encoding the ThsA.

In some embodiments, a Thoeris defense system having an anti-phage activity comprises a nucleic acid construct comprising genes in a set order. In some embodiment, the 5′ to 3′ order of genes is thsA and thsB. In some embodiment, the 5′ to 3′ order of genes in a Thoeris defense system is not thsA and thsB. In some embodiments the 5′ to 3′ order of genes does not affect the anti-phage activity. In some embodiments the 5′ to 3′ order of genes does affect the anti-phage activity.

In some embodiments, the 5′ to 3′ order of genes is random, for example any order of thsA and thsB. In some embodiments, the 5′ to 3′ order of polypeptides is random, for example any order of thsA and 1-10 copies of thsB, wherein thsB may be both 5′ and 3′ of thsA.

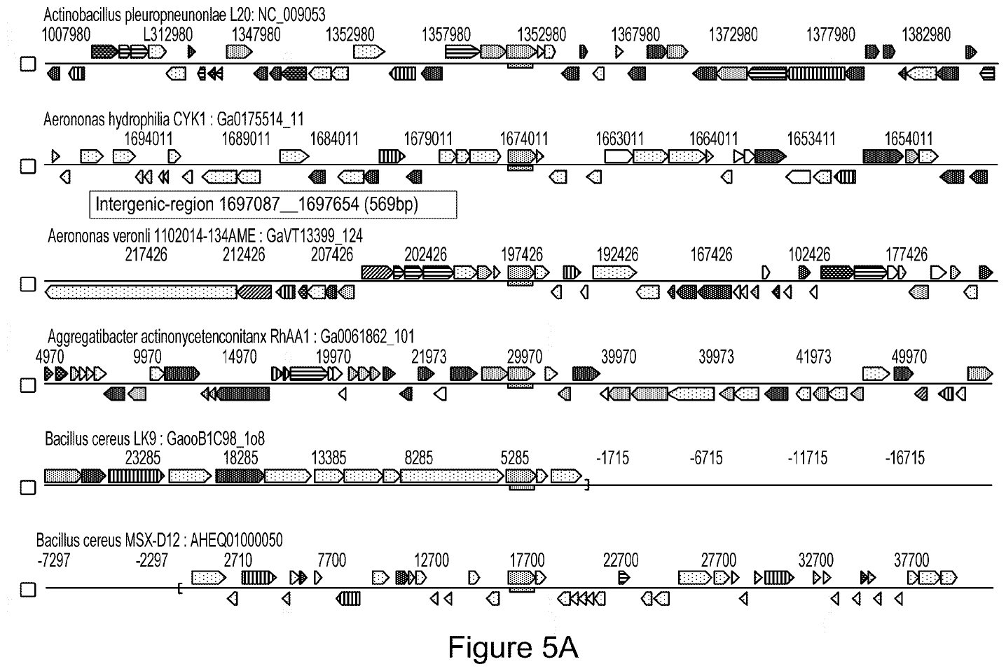

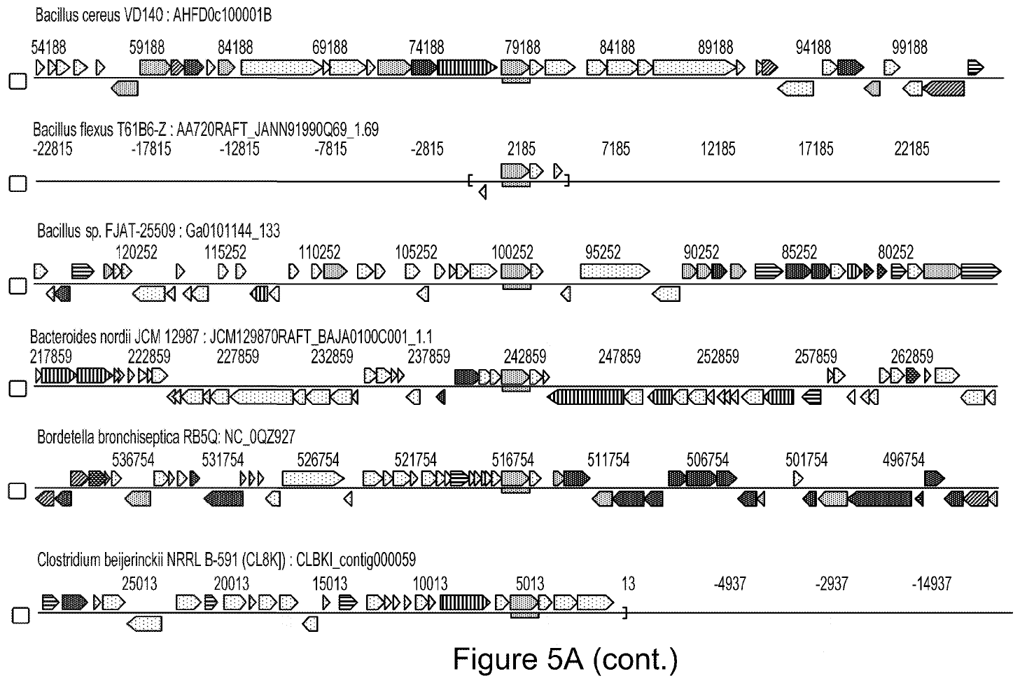

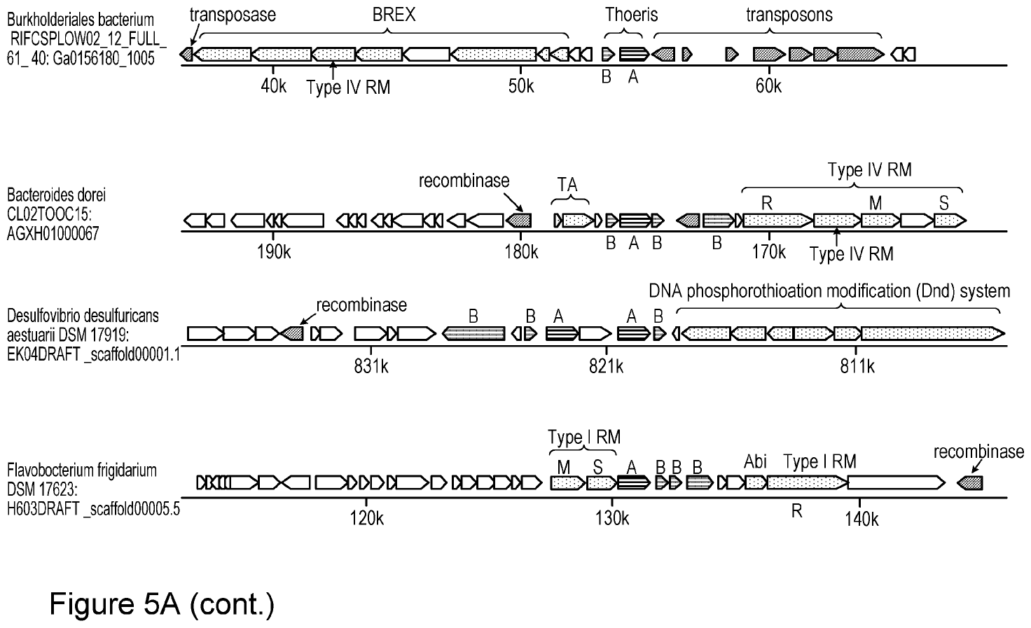

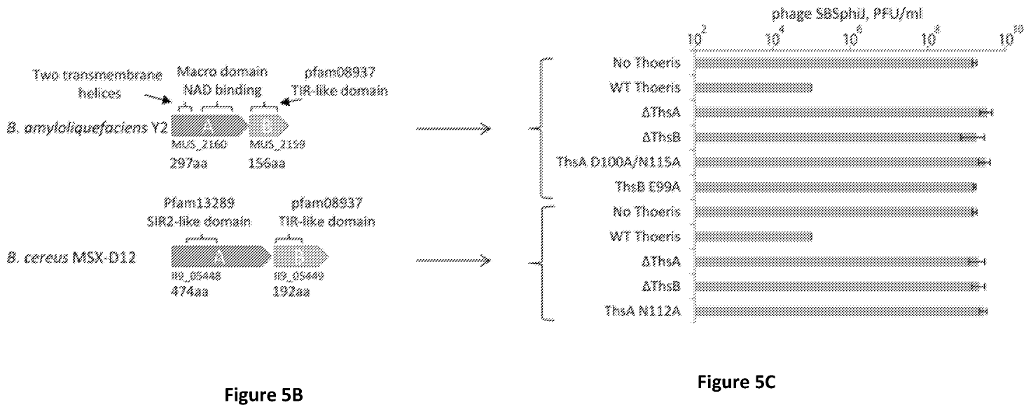

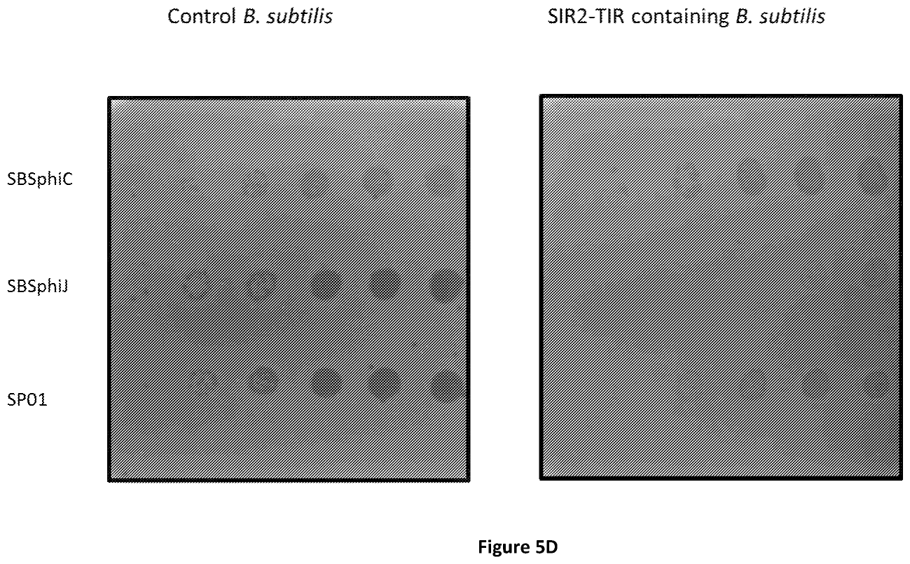

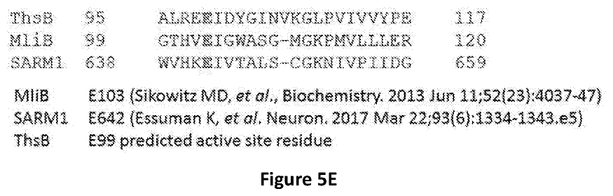

In some embodiments, the Thoeris system (a Defense System II) composition and order is as shown in FIG. 3B or FIG. 5A.

In some embodiments, a Thoeris defense system having an anti-phage activity originates from a microbial genome (Table 9). A skilled artisan would appreciate that the Thoeris system is not present in the majority of bacteria and or archaea species.

In some embodiments, a functional Thoeris defense system comprises a construct comprising nucleic acid sequences encoding polypeptides, wherein the nucleic acid sequence encoding each polypeptide may originate from a different microbial species. For example, the source of the nucleic acid sequence encoding a ThsA polypeptide may be one microbial species, while the source of the nucleic acid sequence encoding a ThsB polypeptide may be a different microbial species. In some embodiments, a functional Thoeris defense system (Defense system II) comprises a non-naturally occurring combination of polypeptide components. In some embodiments, a functional Thoeris defense system (Defense system II) comprises a combination of at least two polypeptides that do not naturally occur together. In some embodiments, a functional Thoeris defense system (Defense system II) comprises a combination of at least three polypeptides that do not naturally occur together.

In some embodiments, a functional Thoeris defense system comprises a construct comprising nucleic acid sequences encoding polypeptides, wherein the nucleic acid sequence encoding each polypeptide may originate from a different bacterial species. For example, the source of the nucleic acid sequence encoding a ThsA polypeptide may be one bacterial species, while the source of the nucleic acid sequence encoding a ThsB polypeptide may be a different bacterial species.

In some embodiments, the source of the nucleic acid encoding a ThsA polypeptide and a ThsB polypeptide is the same. In some embodiments, the source of the nucleic acid encoding a ThsA polypeptide and a ThsB polypeptide is the not the same. A skilled artisan would appreciate that the term “ThsB polypeptide” may in some embodiments, be used interchangeably with “ThsBⁱ polypeptide”, “ThsBⁱⁱ polypeptide”, “ThsBⁱⁱⁱ polypeptide”, “ThsB^{iv} polypeptide”, and “ThsB^{v} polypeptide”, wherein the supra numbers I, ii, iii, iv, and v, indicate additional copies of a ThsB polypeptide comprised in embodiments of a Defense System II. Similarly, skilled artisan would appreciate that the term “thsB gene” may in some embodiments, be used interchangeably with “thsBⁱ gene”, “thsBⁱⁱ gene”, “thsBⁱⁱⁱ gene”, “thsB^{iv} gene”, and “thsB^{v} gene”, wherein the supra numbers i, ii, iii, iv, and v, indicate additional copies of a thsB gene encoding ThsB polypeptides comprised in embodiments of a Defense System II.

In some embodiments, the source of the nucleic acid sequence of any of the components of a Thoeris defense system comprises any of the species listed in Table 9.

In some embodiments, a Thoeris defense system having an anti-phage activity comprises a nucleic acid construct comprising non-coding regions between the nucleic acid sequences encoding the polypeptides. In some embodiments, the non-coding regions between the nucleic acid sequences comprises nucleic acid sequence not naturally occurring in the microbial species of origin. In some embodiments, the non-coding regions between the nucleic acid sequences comprises nucleic acid sequence is the naturally occurring in the microbial species of origin.

In some embodiments, a Thoeris system disclosed herein comprises a multi-gene phage resistance system broadly distributed in microbial genomes, for example but not limited to Proteobacteria, Firmicutes, Bacteriodetes, and Actinobacteria.

According to some embodiments, the Thoeris system components are located in a gene cluster (a cassette of genes) in a microbial cell genome. According to some embodiments, the Thoeris system components are located in close proximity (e.g. positioned within 20 genes, 10 genes, 5 genes or less one from the other) in a genome of a prokaryotic cell. According to some embodiments the prokaryotic cell expresses an endogenous Thoeris defense system. According to some embodiments, the prokaryotic cell expresses an endogenous functional Thoeris defense system. According to some embodiments, the species of prokaryotic cell is selected from the group consisting of the species listed in Table 9. According to some embodiments a prokaryotic cell expresses a non-endogenous Thoeris defense system. According to some embodiments, a prokaryotic cell expresses a non-endogenous functional Thoeris defense system. According to some embodiments, the species of prokaryotic cell expressing a non-endogenous functional Thoeris defense system is selected from the group consisting of the species listed in Table 9.

In some embodiments, Thoeris defense system components comprise ThsA and ThsB polypeptides. In some embodiments, a Thoeris defense system components comprise functional portions of ThsA and ThsB polypeptides. In some embodiments, the Thoeris defense system components are encoded by thsA and thsB genes.

Non-limiting embodiments of endogenous Thoeris systems and the respective location of their components are provided in Table 9 herein.

In some embodiments, the components of a Thoeris system may be identified by the presence of similar domains present within each component. In some embodiments, domains comprise pfam domains and or clusters of orthologous groups (COG) domains.

In some embodiments, a Defense System II comprises a membrane associated complex.

In some embodiments, the term “ThsA” refers to the polynucleotide or expression product e.g., the polypeptide encoded by the thsA gene. In some embodiments, the term “ThsA” refers to a ThsA polypeptide. In some embodiments, the thsA gene encodes a polypeptide comprising a pfam13289 domain. In some embodiments, the thsA gene encodes a polypeptide comprising a pfam14519 domain. In some embodiments, the thsA gene encodes a polypeptide comprising a pfam13289 domain and a pfam14519 domain. In some embodiments, the ThsA polypeptide comprises a pfam13289 domain. In some embodiments, the ThsA polypeptide comprises a pfam14519 domain. In some embodiments, the ThsA polypeptide comprises a pfam13289 domain and a pfam14519 domain.

In some embodiments, the thsA gene encodes a polypeptide comprising a Sir2/Macro domain (NAD binding). In some embodiments, the thsA gene encodes a polypeptide comprising N-terminal transmembrane helices. In some embodiments, the thsA gene encodes a polypeptide comprising a Sir1/Macro domain (NAD binding) and N-terminal transmembrane helices.

In some embodiments, the ThsA polypeptide comprises a Sir2/Macro domain (NAD binding). In some embodiments, the ThsA polypeptide comprises N-terminal transmembrane helices. In some embodiments, the ThsA polypeptide comprises a Sir1/Macro domain (NAD binding) and N-terminal transmembrane helices.

In some embodiments, ThsA polypeptide is encoded by a gene positioned within 5-60 genes of a gene encoding a ThsB polypeptide in a genome of a prokaryotic cell. In some embodiments, ThsA polypeptide is encoded by a gene positioned within 5 genes upstream (5′) or downstream (3′) to a gene encoding a ThsB polypeptide in a genome of a prokaryotic cell. In some embodiments, ThsA polypeptide is encoded by a gene positioned within 5 genes upstream (5′) and downstream (3′) to a gene encoding a ThsB polypeptide in a genome of a prokaryotic cell.

In some embodiments, ThsA and ThsB are encoded by genes positioned sequentially 5′ to 3′ in a genome of a prokaryotic cell. In some embodiments, ThsA and ThsB are encoded by genes positioned contiguously 5′ to 3′ in a genome of a prokaryotic cell. In some embodiments, nucleic acid encoding additional copies of ThsB is positions 3′ of the nucleic acid sequence first encoding a ThsB polypeptide.

In some embodiments, thsA and thsB genes are positioned sequentially 5′ to 3′ in a genome of a prokaryotic cell. In some embodiments, thsA and thsB, genes are positioned contiguously 5′ to 3′ in a genome of a prokaryotic cell. In some embodiments, additional thsB genes are positions 3′ of the first thsB gene, e.g., the order of a gene cassette with 2 copies of a thsB gene would be thsA, thsB, thsB.

In some embodiments, a ThsA polypeptide is about 477 amino acids long (median gene size).

In some embodiments, the ThsA polypeptide comprises the amino acid sequence having at least 80% homology to a sequence selected from the group consisting of the polypeptides referenced in Table 9, rows 2-2100 columns I and J. In some embodiments, the ThsA polypeptide comprises the amino acid sequence having at least 80% homology within similar domain regions within a sequence selected from the group consisting of the polypeptides referenced in Table 9, rows 2-2100 columns I and J. In some embodiments, the ThsA polypeptide comprises a homolog of a polypeptide having the amino acid sequences set forth in the polypeptides referenced in Table 9, rows 2-2100 columns I and J. In some embodiments, a homolog of a ThsA polypeptide comprises a Sir2/Macro domain (NAD binding). In some embodiments, a homolog of ThsA polypeptide comprises N-terminal transmembrane helices. In some embodiments, a homolog of a ThsA polypeptide comprises a Sir1/Macro domain (NAD binding) and N-terminal transmembrane helices.

In some embodiments, the ThsA polypeptide comprises the amino acid sequence having at least 80% identity to a sequence selected from the group consisting of the polypeptides referenced in Table 9, rows 2-2100 columns I and J. In some embodiments, the ThsA polypeptide comprises the amino acid sequence having at least 80% identity within similar domain regions within a sequence selected from the group consisting of the polypeptides referenced in Table 9, rows 2-2100 columns I and J. In some embodiments, the ThsA polypeptide comprises an amino acid sequence having at least 80%, at least 85%, at least 90%, or at least 95% homology to a sequence selected from the group consisting of the polypeptides referenced in Table 9, rows 2-2100 columns I and J. In some embodiments, the ThsA polypeptide comprises the amino acid sequence having at least 80%, 85%, 90%, 95% homology within similar domain regions within a sequence selected from the group consisting of the polypeptides referenced in Table 9, rows 2-2100 columns I and J. In some embodiments, the ThsA polypeptide comprises an amino acid sequence having at least 80%, at least 85%, at least 90%, or at least 95% identity to a sequence selected from the group consisting of the polypeptides referenced in Table 9, rows 2-2100 columns I and J. In some embodiments, the ThsA polypeptide comprises the amino acid sequence having at least 80%, 85%, 90%, 95% identity within similar domain regions within a sequence selected from the group consisting of the polypeptides referenced in Table 9, rows 2-2100 columns I and J. In some embodiments, the ThsA polypeptide comprises an amino acid sequence selected from the group consisting of the polypeptides referenced in Table 9, rows 2-2100 columns I and J.

In some embodiments, the ThsA polypeptide comprises an amino acid sequence encoded by a nucleic acid sequence having at least 80%, at least 85%, at least 90%, or at least 95% homology to a sequence selected from the group consisting of the polynucleotides referenced in Table 9, rows 2-2100 columns I and K. In some embodiments, the ThsA polypeptide comprises the amino acid sequence encoded from a nucleic acid sequence having at least 80%, 85%, 90%, 95% homology within similar domain regions within a sequence selected from the group consisting of the polynucleotides referenced in Table 9, rows 2-2100 columns I and K. In some embodiments, the ThsA polypeptide comprises an amino acid sequence encoded by a nucleic acid sequence having at least 80%, at least 85%, at least 90%, or at least 95% identity to a sequence selected from the group consisting of the polynucleotides referenced in Table 9, rows 2-2100 columns I and K. In some embodiments, the ThsA polypeptide comprises the amino acid sequence encoded from a nucleic acid sequence having at least 80%, 85%, 90%, 95% identity within similar domain regions within a sequence selected from the group consisting of the polynucleotides referenced in Table 9, rows 2-2100 columns I and K. In some embodiments, the ThsA polypeptide comprises an amino acid sequence encoded from a nucleic acid sequence selected from the group consisting of the polynucleotides referenced in Table 9, rows 2-2100 columns I and K.

In some embodiments, the nucleic acid sequence of a thsA gene comprises a nucleic acid sequence having at least 80%, at least 85%, at least 90%, or at least 95% homology to a sequence selected from the group consisting of the polynucleotides referenced in Table 9, rows 2-2100 columns I and K. In some embodiments, the nucleic acid sequence of a thsA gene comprises a nucleic acid sequence having at least 80%, at least 85%, at least 90%, or at least 95% identity to a sequence selected from the group consisting of the polynucleotides referenced in Table 9, rows 2-2100 columns I and K. In some embodiments, the nucleic acid sequence of a thsA gene comprises a nucleic acid sequence having at least 80%, at least 85%, at least 90%, or at least 95% homology within the nucleotide sequences encoding similar domain sequences with a sequence selected from the group consisting of the polynucleotides referenced in Table 9, rows 2-2100 columns I and K. In some embodiments, the nucleic acid sequence of a thsA gene comprises a nucleic acid sequence having at least 80%, at least 85%, at least 90%, or at least 95% identity within nucleotide sequences encoding similar domain regions with a sequence selected from the group consisting of the polynucleotides referenced in Table 9, rows 2-2100 columns I and K. In some embodiments, the nucleic acid sequence of a thsA gene comprises a nucleic acid sequence selected from the group consisting of the polynucleotides referenced in Table 9, rows 2-2100 columns I and K.

As used herein, the term “ThsB” refers to the polynucleotide or expression product e.g., polypeptide encoded by the thsB gene. In some embodiments, the term “ThsB” refers to a ThsB polypeptide. In some embodiments, the product of the thsB gene comprises a pfam08937 domain or a pfam08357 domain. In some embodiments, the thsB gene encodes a Toll/interleukin-1 receptor (TIR) domain.

In some embodiments, ThsB polypeptide is encoded by a gene positioned within 5-60 genes of a gene encoding a ThsA polypeptide in a genome of a prokaryotic cell. In some embodiments, ThsB polypeptide is encoded by a gene positioned within 5 genes upstream (5′) or downstream (3′) to a gene encoding a ThsA polypeptide in a genome of a prokaryotic cell. In some embodiments, ThsB polypeptide is encoded by a gene positioned within 5 genes upstream (5′) and downstream (3′) to a gene encoding a ThsA polypeptide in a genome of a prokaryotic cell.

In some embodiments, ThsA and ThsB are encoded by genes positioned sequentially 5′ to 3′ in a genome of a prokaryotic cell. In some embodiments ThsA and ThsB are encoded by genes positioned contiguously 5′ to 3′ in a genome of a prokaryotic cell. In some embodiments, additional ThsB polypeptides are encoded by genes positioned 3′ of the first gene encoding ThsB. In some embodiments ThsA and ThsB (1-10 copies) are encoded by genes positioned contiguously 5′ to 3′ in a genome of a prokaryotic cell.

In some embodiments, a ThsB polypeptide is about 195 amino acids long (median gene size).

In some embodiments, the ThsB polypeptide comprises an amino acid sequence having at least 80% homology to a sequence selected from the group consisting of the polypeptides referenced in Table 9, rows 2-2100 columns M and N, Q and R, U and V, Y and Z, and AC and AD. In some embodiments, the ThsB polypeptide comprises the amino acid sequence having at least 80%, 85%, 90%, 95% homology within similar domain regions within a sequence selected from the group consisting of the polypeptides referenced in Table 9, rows 2-2100 columns M and N, Q and R, U and V, Y and Z, and AC and AD. In some embodiments, a homologue of the ThsB polypeptide comprises a TIR domain. In some embodiments, the ThsB polypeptide comprises an amino acid sequence having at least 80% identity to a sequence selected from the group consisting of the polypeptides referenced in Table 9, rows 2-2100 columns M and N, Q and R, U and V, Y and Z, and AC and AD.

In some embodiments, the ThsB polypeptide comprises an amino acid sequence having at least 80%, at least 85%, at least 90%, or at least 95% homology to a sequence selected from the group consisting of the polypeptides referenced in Table 9, rows 2-2100 columns M and N, Q and R, U and V, Y and Z, and AC and AD. In some embodiments, the ThsB polypeptide comprises the amino acid sequence having at least 80%, 85%, 90%, 95% homology within similar domain regions within a sequence selected from the group consisting of the polypeptides referenced in Table 9, rows 2-2100 columns M and N, Q and R, U and V, Y and Z, and AC and AD. In some embodiments, the ThsB polypeptide comprises an amino acid sequence having at least 80%, at least 85%, at least 90%, or at least 95% identity to a sequence selected from the group consisting of the polypeptides referenced in Table 9, rows 2-2100 columns M and N, Q and R, U and V, Y and Z, and AC and AD. In some embodiments, the ThsB polypeptide comprises the amino acid sequence having at least 80%, 85%, 90%, 95% identity within similar domain regions within a sequence selected from the group consisting of the polypeptides referenced in Table 9, rows 2-2100 columns M and N, Q and R, U and V, Y and Z, and AC and AD. In some embodiments, the ThsB polypeptide comprises an amino acid sequence selected from the group consisting of the polypeptides referenced in Table 9, rows 2-2100 columns M and N, Q and R, U and V, Y and Z, and AC and AD.

In some embodiments, the ThsB polypeptide comprises an amino acid sequence encoded from a nucleic acid sequence having at least 80%, at least 85%, at least 90%, or at least 95% homology to a sequence selected from the group consisting of the polynucleotides referenced in Table 9, rows 2-2100 columns M and O, Q and S, U and W, Y and AA, and AC and AE. In some embodiments, the ThsB polypeptide comprises the amino acid sequence encoded by a nucleic acid sequence having at least 80%, 85%, 90%, 95% homology within similar domain regions within a sequence selected from the group consisting of the polynucleotides referenced in Table 9, rows 2-2100 columns M and O, Q and S, U and W, Y and AA, and AC and AE. In some embodiments, the ThsB polypeptide comprises an amino acid sequence encoded from a nucleic acid sequence having at least 80%, at least 85%, at least 90%, or at least 95% identity to a sequence selected from the group consisting of the polynucleotides referenced in Table 9, rows 2-2100 columns M and O, Q and S, U and W, Y and AA, and AC and AE. In some embodiments, the ThsB polypeptide comprises the amino acid sequence encoded by a nucleic acid sequence having at least 80%, 85%, 90%, 95% identity within similar domain regions within a sequence selected from the group consisting of the polynucleotides referenced in Table 9, rows 2-2100 columns M and O, Q and S, U and W, Y and AA, and AC and AE. In some embodiments, the ThsB polypeptide comprises an amino acid sequence encoded from a nucleic acid sequence selected from the group consisting of the polynucleotides referenced in Table 9, rows 2-2100 columns M and O, Q and S, U and W, Y and AA, and AC and AE.

In some embodiments, the nucleic acid sequence of a thsB gene comprises a nucleic acid sequence having at least 80%, at least 85%, at least 90%, or at least 95% homology to a sequence selected from the group consisting of the polynucleotides referenced in Table 9, rows 2-2100 columns M and O, Q and S, U and W, Y and AA, and AC and AE. In some embodiments, the nucleic acid sequence of a thsB gene comprises a nucleic acid sequence having at least 80%, at least 85%, at least 90%, or at least 95% identity to a sequence selected from the group consisting of the polynucleotides referenced in Table 9, rows 2-2100 columns M and O, Q and S, U and W, Y and AA, and AC and AE. In some embodiments, the nucleic acid sequence of a thsB gene comprises a nucleic acid sequence having at least 80%, at least 85%, at least 90%, or at least 95% homology within the nucleotide sequences encoding similar domain sequences with a sequence selected from the group consisting of the polynucleotides referenced in Table 9, rows 2-2100 columns M and O, Q and S, U and W, Y and AA, and AC and AE. In some embodiments, the nucleic acid sequence of a thsB gene comprises a nucleic acid sequence having at least 80%, at least 85%, at least 90%, or at least 95% identity within nucleotide sequences encoding similar domain regions with a sequence selected from the group consisting of the polynucleotides referenced in Table 9, rows 2-2100 columns M and O, Q and S, U and W, Y and AA, and AC and AE. In some embodiments, the thsB gene homolog encodes a polypeptide comprising a helicase activity. In some embodiments, the thsB gene homolog encodes a polypeptide comprising a DEAx box helicase activity. In some embodiments, the nucleic acid sequence of a thsB gene comprises a nucleic acid sequence selected from the group consisting of the polynucleotides referenced in Table 9, rows 2-2100 columns M and O, Q and S, U and W, Y and AA, and AC and AE.

A skilled artisan would appreciate that a “functional portion of a Defense System II component” or “functional fragment of Defense System II component” or “functional portion of a Thoeris defense system component” or “functional fragment of Thoeris defense system component” refers to a functional portion of a Thoeris polynucleotide or polypeptide, as disclosed herein, which expression is sufficient to elicit an anti-phage activity alone or in combination with the at least one of the other Thoeris polynucleotides or polypeptides disclosed herein or functional portions thereof.

The terms “ThsA”, “ThsB”, “thsA”, and “thsB”, also refer to functional ThsA, ThsB, thsA, and thsB, homologs, which exhibit the desired activity (i.e., conferring phage resistance). Such homologs can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide sequences referenced in Table 9, rows 2-2100 columns I and J, M and N, Q and R, U and V, Y and Z, and SAC and AD, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequences referenced in Table 9, rows 2-2100 columns I and K, M and O, Q and S, U and W, Y and AA, and SAC and AE, respectively. In some embodiments, such homologs comprise at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% homology within similar domain regions of the polypeptide sequences referenced in Table 9, rows 2-2100 columns I and J, M and N, Q and R, U and V, Y and Z, and AC and AD, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity within nucleotide sequences encoding similar domain regions to the polynucleotide sequences referenced in Table 9, rows 2-2100 columns I and K, M and O, Q and S, U and W, Y and AA, and AC and AE, respectively.

Table 9 presents embodiments of components of Defense System II that may be found in a diverse array of bacteria and archaea genomes (referenced in Table 18). Table 9 presents embodiments of nucleic acid sequences encoding gene cassettes of Defense System II (referenced in Table 18).

The Druantia Defense System

In some embodiments, a defense system disclosed herein comprises an Druantia anti-phage defense system. (Table 10) In some embodiments, a Druantia defense system provides a host cell with resistance to entry of foreign nucleic acid invasion. In some embodiments, a host cell expressing a functional Druantia defense system (Defense System IIIa; Defense System IIIb; or Defense System IIIc) provides the host cell resistance foreign nucleic acid invasion.

In some embodiments, a Druantia defense system provides a host cell with resistance to at least one phage.

In some embodiments, a Druantia defense system provides a host cell with resistance to phage infection. In some embodiments, expression of a Druantia defense system in bacteria protects the bacteria from phage infection.

As used herein, the term “a Druantia anti-phage defense system” may be used interchangeably with the term “a Defense System III”, having all the same meanings and qualities. A skilled artisan would appreciate that the term “Druantia system” may be used interchangeably in some embodiments with “Druantia defense system”, “Druantia the defense system”, “Druantia anti-phage system”, and “Defense System III”, having all the same meanings and qualities. Disclosed herein, the Druantia defense system (Defense System III) comprises a Type I Druantia defense system (Defense System IIIa), a Type II Druantia defense system (Defense System IIIb), and a Type III Druantia defense system (Defense system IIIc), as described below.

In some embodiments, expression of a Druantia Type I defense system (Defense System IIIa) in bacteria protects the bacteria from phage infection. In some embodiments, expression of a Druantia Type II defense system (Defense System IIIb) in bacteria protects the bacteria from phage infection. In some embodiments, expression of a Druantia Type III defense system (Defense System IIIc) in bacteria protects the bacteria from phage infection.

In some embodiments, expression of a Druantia defense system in bacteria protects the bacteria from phage infection.

In some embodiments, a Druantia defense system (Defense System IIIa or Defense system IIIb or Defense System IIIc) provides a host cell with resistance to plasmid transformation. In some embodiments, a host cell expressing a functional Druantia defense system (Defense System IIIa or Defense System IIIb or Defense System IIIc) provides a host cells with resistance to plasmid transformation.

In some embodiments, a Druantia defense system (Defense system IIIa or IIIb or IIIc) provides a host cell with resistance to entry of conjugative elements. In some embodiments, a host cell expressing a function Druantia defense system (Defense System IIIa or IIIb or IIIc) provides the host cell resistance from entry of conjugative elements.

In some embodiments, a microbial species does not comprise an endogenous Defense System III. In some embodiments, a microbial species does not express an endogenous Defense System III. In some embodiments, a microbial species does not express an endogenous functional Defense System III.

In some embodiments, a bacterial species does not comprise an endogenous Defense System III. In some embodiments, a bacterial species does not express an endogenous Defense System III. In some embodiments, a bacterial species does not express an endogenous functional Defense System III.

In some embodiments, a microbial species does not comprise an endogenous Defense System IIIa. In some embodiments, a microbial species does not express an endogenous Defense System IIIa. In some embodiments, a microbial species does not express an endogenous functional Defense System IIIa.

In some embodiments, a bacterial species does not comprise an endogenous Defense System IIIA. In some embodiments, a bacterial species does not express an endogenous Defense System IIIa. In some embodiments, a bacterial species does not express an endogenous functional Defense System IIIa.

In some embodiments, a microbial species does not comprise an endogenous Defense System IIIb. In some embodiments, a microbial species does not express an endogenous Defense System IIIb. In some embodiments, a microbial species does not express an endogenous functional Defense System IIIb.

In some embodiments, a bacterial species does not comprise an endogenous Defense System IIIb. In some embodiments, a bacterial species does not express an endogenous Defense System IIIb. In some embodiments, a bacterial species does not express an endogenous functional Defense System IIIb.

In some embodiments, a microbial species does not comprise an endogenous Defense System IIIc. In some embodiments, a microbial species does not express an endogenous Defense System IIIc. In some embodiments, a microbial species does not express an endogenous functional Defense System IIIc.

In some embodiments, a bacterial species does not comprise an endogenous Defense System IIIc. In some embodiments, a bacterial species does not express an endogenous Defense System IIIc. In some embodiments, a bacterial species does not express an endogenous functional Defense System IIIc.

In some embodiments, a Druantia Type I defense system (Defense System IIIa) having an anti-phage activity comprises a nucleic acid construct comprising a nucleic acid sequence encoding a DruA polypeptide comprising a DUF4338 domain or a pfam14236 domain or a combination thereof, a DruB polypeptide, a DruC polypeptide, a DruD polypeptide, and a DruE polypeptide comprising a pfam00270 domain or a pfam00271 domain or a pfam09369 domain or a DUF1998 domain or any combination thereof. In some embodiments, a Druantia Type I defense system (Defense System IIIa) having an anti-phage activity comprises a nucleic acid construct comprising a nucleic acid sequence encoding a DruA polypeptide comprising a DUF4338 domain or a pfam14236 domain or a combination thereof, a DruB polypeptide wherein said DruB polypeptide is encoded by a gene positioned within 5 genes of a gene encoding said DruA polypeptide and said DruE polypeptide, a DruC polypeptide wherein said DruC polypeptide is encoded by a gene positioned within 5 genes of a gene encoding said DruA polypeptide and said DruE polypeptide, a DruD polypeptide wherein said DruD polypeptide is encoded by a gene positioned within 5 genes of a gene encoding said DruA polypeptide and said DruE polypeptide, and a DruE polypeptide comprising a pfam00270 domain or a pfam00271 domain or a pfam09369 domain or a DUF1998 domain or any combination thereof.

In some embodiments, a Druantia Type I defense system (Defense System IIIa) having an anti-phage activity comprises a DruA polypeptide comprising a DUF4338 domain or a pfam14236 domain or a combination thereof, a DruB polypeptide, a DruC polypeptide, a DruD polypeptide, and a DruE polypeptide comprising a pfam00270 domain or a pfam00271 domain or a pfam09369 domain or a DUF1998 domain or any combination thereof. In some embodiments, a Druantia Type I defense system (Defense System IIIa) having an anti-phage activity comprises a DruA polypeptide comprising a DUF4338 domain or a pfam14236 domain or a combination thereof, a DruB polypeptide wherein said DruB polypeptide is encoded by a gene positioned within 5 genes of a gene encoding said DruA polypeptide and said DruE polypeptide, a DruC polypeptide wherein said DruC polypeptide is encoded by a gene positioned within 5 genes of a gene encoding said DruA polypeptide and said DruE polypeptide, a DruD polypeptide wherein said DruD polypeptide is encoded by a gene positioned within 5 genes of a gene encoding said DruA polypeptide and said DruE polypeptide, and a DruE polypeptide comprising a pfam00270 domain or a pfam00271 domain or a pfam09369 domain or a DUF1998 domain or any combination thereof.

In some embodiments, a Defense System IIIa comprises a DruA polypeptide comprising a DUF4338 domain or a pfam14236 domain or any combination thereof, or comprising an amino acid sequence having at least 80% identity to a sequence selected from the group referenced in Table 10 rows 2-123 columns J and K; or a DruB polypeptide comprising an amino acid sequence having at least 80% identity to a sequence selected from the group referenced in Table 10 rows 2-123 columns N and O; or a DruC polypeptide comprising an amino acid sequence having at least 80% identity to a sequence selected from the group referenced in Table 10 rows 2-123 columns R and S; or a DruD polypeptide comprising an amino acid sequence having at least 80% identity to a sequence selected from the group referenced in Table 10 rows 2-123 columns V and W; and a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA; or any combination thereof.

In some embodiments, a Defense System IIIa comprises at least two polypeptide components selected from a DruA polypeptide comprising a DUF4338 domain or a pfam14236 domain or any combination thereof, or comprising an amino acid sequence having at least 80% identity to a sequence selected from the group referenced in Table 10 rows 2-123 columns J and K; or a DruB polypeptide comprising an amino acid sequence having at least 80% identity to a sequence selected from the group referenced in Table 10 rows 2-123 columns N and O; or a DruC polypeptide comprising an amino acid sequence having at least 80% identity to a sequence selected from the group referenced in Table 10 rows 2-123 columns R and S; or a DruD polypeptide comprising an amino acid sequence having at least 80% identity to a sequence selected from the group referenced in Table 10 rows 2-123 columns V and W; and a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA. In some embodiments, a Defense System IIIa comprises at least three polypeptide components selected from a DruA polypeptide comprising a DUF4338 domain or a pfam14236 domain or any combination thereof, or comprising an amino acid sequence having at least 80% identity to a sequence selected from the group referenced in Table 10 rows 2-123 columns J and K; or a DruB polypeptide comprising an amino acid sequence having at least 80% identity to a sequence selected from the group referenced in Table 10 rows 2-123 columns N and O; or a DruC polypeptide comprising an amino acid sequence having at least 80% identity to a sequence selected from the group referenced in Table 10 rows 2-123 columns R and S; or a DruD polypeptide comprising an amino acid sequence having at least 80% identity to a sequence selected from the group referenced in Table 10 rows 2-123 columns V and W; and a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 column Z and AA. In some embodiments, a Defense System IIIa comprises at least four polypeptide components selected from a DruA polypeptide comprising a DUF4338 domain or a pfam14236 domain or any combination thereof, or comprising an amino acid sequence having at least 80% identity to a sequence selected from the group referenced in Table 10 rows 2-123 columns J and K; or a DruB polypeptide comprising an amino acid sequence having at least 80% identity to a sequence selected from the group referenced in Table 10 rows 2-123 columns N and O; or a DruC polypeptide comprising an amino acid sequence having at least 80% identity to a sequence selected from the group referenced in Table 10 rows 2-123 columns R and S; or a DruD polypeptide comprising an amino acid sequence having at least 80% identity to a sequence selected from the group referenced in Table 10 rows 2-123 columns V and W; and a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA. In some embodiments, a Defense System IIIa comprises five polypeptide components selected from a DruA polypeptide comprising a DUF4338 domain or a pfam14236 domain or any combination thereof, or comprising an amino acid sequence having at least 80% identity to a sequence selected from the group referenced in Table 10 rows 2-123 columns J and K; or a DruB polypeptide comprising an amino acid sequence having at least 80% identity to a sequence selected from the group referenced in Table 10 rows 2-123 columns N and O; or a DruC polypeptide comprising an amino acid sequence having at least 80% identity to a sequence selected from the group referenced in Table 10 rows 2-123 columns R and S; or a DruD polypeptide comprising an amino acid sequence having at least 80% identity to a sequence selected from the group referenced in Table 10 rows 2-123 columns V and W; and a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA.

In some embodiments, a functional Defense System IIIa comprises at least two polypeptide components selected from a DruA polypeptide comprising a DUF4338 domain or a pfam14236 domain or any combination thereof, or comprising an amino acid sequence having at least 80% identity to a sequence selected from the group referenced in Table 10 rows 2-123 columns J and K; or a DruB polypeptide comprising an amino acid sequence having at least 80% identity to a sequence selected from the group referenced in Table 10 rows 2-123 columns N and O; or a DruC polypeptide comprising an amino acid sequence having at least 80% identity to a sequence selected from the group referenced in Table 10 rows 2-123 columns r and S; or a DruD polypeptide comprising an amino acid sequence having at least 80% identity to a sequence selected from the group referenced in Table 10 rows 2-123 columns V and W; and a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA. In some embodiments, a functional Defense System IIIa comprises at least three polypeptide components selected from a DruA polypeptide comprising a DUF4338 domain or a pfam14236 domain or any combination thereof, or comprising an amino acid sequence having at least 80% identity to a sequence selected from the group referenced in Table 10 rows 2-123 columns J and K; or a DruB polypeptide comprising an amino acid sequence having at least 80% identity to a sequence selected from the group referenced in Table 10 rows 2-123 columns N and O; or a DruC polypeptide comprising an amino acid sequence having at least 80% identity to a sequence selected from the group referenced in Table 10 rows 2-123 columns R and S; or a DruD polypeptide comprising an amino acid sequence having at least 80% identity to a sequence selected from the group referenced in Table 10 rows 2-123 columns V and W; and a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA. In some embodiments, a functional Defense System IIIa comprises at least four polypeptide components selected from a DruA polypeptide comprising a DUF4338 domain or a pfam14236 domain or any combination thereof, or comprising an amino acid sequence having at least 80% identity to a sequence selected from the group referenced in Table 10 rows 2-123 columns J and K; or a DruB polypeptide comprising an amino acid sequence having at least 80% identity to a sequence selected from the group referenced in Table 10 rows 2-123 columns N and O; or a DruC polypeptide comprising an amino acid sequence having at least 80% identity to a sequence selected from the group referenced in Table 10 rows 2-123 columns R and S; or a DruD polypeptide comprising an amino acid sequence having at least 80% identity to a sequence selected from the group referenced in Table 10 rows 2-123 columns V and W; and a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA. In some embodiments, a functional Defense System IIIa comprises five polypeptide components selected from a DruA polypeptide comprising a DUF4338 domain or a pfam14236 domain or any combination thereof, or comprising an amino acid sequence having at least 80% identity to a sequence selected from the group referenced in Table 10 rows 2-123 columns J and K; or a DruB polypeptide comprising an amino acid sequence having at least 80% identity to a sequence selected from the group referenced in Table 10 rows 2-123 columns N and O; or a DruC polypeptide comprising an amino acid sequence having at least 80% identity to a sequence selected from the group referenced in Table 10 rows 2-123 columns R and S; or a DruD polypeptide comprising an amino acid sequence having at least 80% identity to a sequence selected from the group referenced in Table 10 rows 2-123 columns V and W; and a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA.

In some embodiments, a Druantia Type I defense system (Defense System IIIa) having an anti-phage activity comprises a nucleic acid construct comprising a nucleic acid sequence comprising a druA gene, a druB gene, a druC gene, a druD gene, and a druE gene.

In some embodiments, the Druantia Type I anti-phage defense system comprises a nucleic acid construct comprising nucleic acid sequences encoding a gene cassette comprising druA, druB, druC, druD, and druE genes. In some embodiments, a Druantia Type I (Defense System IIIa) gene cassette comprises the nucleic acid sequence:

(SEQ ID NO: 16; construct 56 of Table 4)

| | |
|---|---|
| 1 | tagtaaggca ttatagtgaa acgatgtcta tgaatcacac atctaatgtc cggtgaacgt |
| 61 | ttggttgata gggtagtaaa actagtaatc atcctataat tagctatatt cgtggttatt |
| 121 | agattgaaaa cagataacat taacaaaatc tataaatcta tttgaatgat ttttttcatc |
| 181 | aatgctgttg taacctcctg ctatcaaaag tttttcacaa aatctataag ctcccagaat |
| 241 | tgcttgtata aatgctatca ttggcgctgt cccgatcgag ggagcaagga ggggactctc |
| 301 | ttgtgccatg cgattaatca cttgggctct aagtgaaatt tagtgggact aaatactaat |
| 361 | tggaacgtga gataaaaatg cacaaagatc cctctataat agttaatatc aaccttcgag |
| 421 | aagccaaact gaaaaagaag gtacgtgagc atttacaatc cttgggtttt acaagatctg |
| 481 | attctggagc gctccaggcc ccgggaaata ccaaagatgt aatacgggct cttcatagtt |
| 541 | ctcaacgagc tgagcggata tttgcaaacc aaaagttcat aacgctaaga gcggcaaagc |
| 601 | ttattaaatt tttcgcatcc ggcaatgagg tcattccgga taaaatttca ccggtacttg |
| 661 | aacgtgtaaa gtcaggaacc tggcaaggag atctctttag gttagcagca ttaacttggt |
| 721 | ccgtacctgt ttcaagcgga tttggaaggc gtctccggta tcttgtatgg gatgaaagca |
| 781 | acggaaaatt gatagggctg atcgcaattg gtgaccctgt gttcaacctt gcagtccgag |
| 841 | ataatttgat tgggtgggat actcatgcca gaagttcccg gcttgttaat ttgatggatg |
| 901 | catacgtcct cggtgctctt cccccttata atgccctgct gggaggaaaa ttaattgcat |
| 961 | gtctgcttcg tagccgcgat ctttatgatg actttgcaaa ggtctatggt gataccgttg |
| 1021 | gagtaatatc tcaaaaaaag aaacaagcac gtcttttggc tattacaaca acatcgtcta |
| 1081 | tggggcgctc atcggtatat aaccgtttaa agctggatgg aattcaatat ttaaaatcga |
| 1141 | ttggatatac aggcggttgg gggcattttc atatacctga tagcttgttc attgaattac |
| 1201 | gtgattactt acgtgatatg gatcacgctt atgcagatca ttatatgttt ggtaatgggc |
| 1261 | ctaactggcg tttacgtaca actaaggcag ctttaaatgt actaggattt agagataatt |
| 1321 | tgatgaagca tggaattcaa cgtgaagttt ttatcagtca gctagcagaa aatgcaacta |
| 1381 | gtattctgca aacaggcaaa ggtgaaccag atctaacctc tttgctttct gctaaagaga |
| 1441 | tagctgagtg tgcgatggca cgatggatgg ttccacgatc aattcgcaat ccagaatatc |
| 1501 | ggctttggaa agcaagagat ctatttgatt ttattagtaa tgactcgcta aaatttcccc |
| 1561 | cgtctgacga gatagcgaaa acagttgtct aatcttaact gaagggggag taagtgaatt |
| 1621 | acgctattga taagttcacc gggacactga tattagcagc tcgagcaacg aaatatgctc |
| 1681 | aatatgtttg cccagtttgt aaaaaaggtg ttaacctccg taaagggaag gttatacccc |
| 1741 | catattttgc tcatttgccc ggacatggta cgtcagactg tgaaaatttt gttcccggaa |
| 1801 | attccatcat tatcgaaact attaaaacta tttcaaagcg atatatggat ttgcgcttat |
| 1861 | tgattcctgt cggaagtaat agtcgagagt ggtcattaga attagtgttg ccaacctgta |
| 1921 | atttatgtag agcaaagata acgttagatg taggaggcag aagccaaacg cttgatatga |
| 1981 | ggagtatggt aaagagtcgc cagattggtg ctgaattatc agtaaaatct taccgtattg |
| 2041 | tttcatatag tggtgaaccc gatccaaaat ttgtaacaga agttgaaaga gaatgccccg |
| 2101 | gtttaccttc tgagggagca gcagttttca ctgctttagg gcgtggggca tcgaagggat |
| 2161 | ttccacgagc acaagagtta agatgtactg aaacatttgc ctttctttgg cgacaccctg |
| 2221 | ttgctccaga ttttcctgat gaattagaaa taaaaagttt agctagtaaa cagggatgga |
| 2281 | atttagctct tgttacaatt cctgaagtcc cttctgtgga gagtatttca tggctaaaat |
| 2341 | cttttacata ccttcctgtt gttcctgcca gaacatctat tacagcaatt tggccgttcc |
| 2401 | taaatcaaaa aacaagtatt aatcatgtcg aatgtgttca ttctgacaca atattgttgt |
| 2461 | cagcaaatat ggcaccaaca tcatcagaaa atgttggacc aactatgtac gcacaaggtt |
| 2521 | cctctttatt actttcagcg gttggtgttg aaaaatcacc tgctttcttc attcttaatc |
| 2581 | ctggagaaaa tgactttgtg ggcgtttctg gctcaattga gcaggacgta aacttatttt |
| 2641 | tttctttcta taaaaaaaac gtttctgtac ccagaaaata tccctcaata gatttggttt |
| 2701 | ttactaagag gaataaagaa aagaccatcg tttccttaca tcaaagaaga tgcattgaag |
| 2761 | ttatgatgga agcacgaatg tttggccata aattagaata catgtctatg ccttctggtg |
| 2821 | ttgaaggagt ggcaagaatt caaagacaaa ctgaaagtag tgttattaag ttagtttcta |
| 2881 | atgatgacat tgcagctcat gataagagca tgcggttact atctcctgtt gcgttatctc |
| 2941 | aattatctga ttgcttagca aacttaacat gtcatgtaga aatagatttt ttaggtcttg |
| 3001 | gtaaaatatt tttacctagt tcttctatgt tatcattaga tgacggggaa tttattgaat |
| 3061 | tatctcctaa tcttcgctca cggatattaa gttttatact tcaaatgggg cacaccctcc |
| 3121 | atggttttag tttaaataat gattttttat tagttgagaa attagtggat ttgcagccgg |
| 3181 | aaccacactt attaccgcat tatagggcat tggtaaaaga agttaagacc aatggatttg |
| 3241 | aatgtaaccg ctttagataa ggtgccttcg aatgagttac caatatagcc aagaggcaaa |
| 3301 | ggaacggatc tctaagttgg gacaatccga aattgttaac tttatcaatg agatttctcc |
| 3361 | aactttacga cgtaaagctt ttggttgttt accaaaagta ccgggattca gggcaggaca |
| 3421 | tcccactgaa attaaagaaa aacagaaaag attgattggg tatatgttcc agtcacatcc |
| 3481 | ttcctctgag gagagaaaag catggaaaag tttttctctt ttttggcagt tttgggctga |
| 3541 | agagaaaatt gacaaatcat ttagtatgat tgataattta ggattaaaag aaaactctgg |
| 3601 | ctctattttt attagagagc ttgctaaaaa ctttcctaaa gttgctagag agaatatcga |
| 3661 | gcgcctgttt atctttagtg ggtttgctga tgatccagac gttataaatg catttaacct |
| 3721 | ttttcctcct gcagttgttc ttgcccgcga tatcgtggtt gatactcttc caattcgttt |
| 3781 | agatgagctt gaagcacgta ttagtttaat tgccgataat gtggagaaaa aaaataacca |
| 3841 | tattaaagag cttgagttaa aaatagatgc tttttccgaa cggttcgata attactttaa |
| 3901 | taatgaaaag agcaatttaa aaataattaa cgaactacaa tccttaataa actcagagac |
| 3961 | taaacaatct gatattgcta ataaatctat tgacgagctt tatcatttta atgaaaaaaa |
| 4021 | caaacagcta atattatctc ttcaagagaa attagatttt aatgctctgg ctatgaatga |
| 4081 | catttctgag catgaaaaat tgataaaaag tatggctaat gaaatttcag agttaaaaaa |
| 4141 | tgcattaact atcttgtgtg ataataaaag aaagaataac gagttagatt atatcaatga |
| 4201 | attaaaaaaa ctcactgaac gaatagatac acttgaaata aacacatctc aagctagcaa |
| 4261 | agtgagtgtc accaatagat ttacaaaatt ccatgaaata gcgcactatg aaaattatga |
| 4321 | atatctttca tcctccgaag acatatctaa tagaatttct ttaaatttac aggctgttgg |
| 4381 | gttaacaaaa aattcagcag aaacattggc tagattgaca ttagctacct tcgtttctgg |
| 4441 | acaaatcatt caattcagtg gctctttggc agatattatc gcggatgcaa ttgccattgc |
| 4501 | tattggtgca ccacgttatc acatatggag agttccagtt ggtattattt ctgacatgga |
| 4561 | ttcttttgat tttatagaga ctatagctga atcatctcgc tgtctccttt tgaaaggggc |
| 4621 | caatctttca gcatttgaga tttatggagc ggcaattaga gatatagttg ttcaacggca |
| 4681 | aatacatcca acaaattatg accatctggc attgatagct acttggaaac aaggccctgc |
| 4741 | tacattccct gatggaggaa tgttggccga gttgggacca gttattgata ctgatacatt |
| 4801 | aaaaatgcgt ggtttatctg ctattttacc ccaattgaaa ccaggttgtc ttgccaagga |
| 4861 | taaatggaca aatattgatg gactacagct tgatagtgtt gatgattatg tagatgaatt |
| 4921 | aagagcatta ctggacgaag ctggatttga tgggggaact ttgtggaaga gaatggttca |
| 4981 | tattttctat acttcactca taagagtccc taatggaaat tatatttatg atctttattc |
| 5041 | tgtcttgtct ttttatactc ttacatgggc aaaaattaaa ggtggccccg tccaaaagat |
| 5101 | agaagatatt gccaatcgtg aattaaaaaa ttatagtgca aaaatatctt cttgaggagg |
| 5161 | tggttaatgg agtggagagc agtatcacga gacaaagcac tggatatgtt atcaactgca |
| 5221 | ttaaattgtc gatttgatga tgaagggttg agaatttcag cagtttcaga atgcttaagg |
| 5281 | agcgtattat atcaatattc tatatctgaa acagaagaag ctaggcaaac tgtaacctcg |
| 5341 | cttcgactca ctagtgcagt aaggcgaaaa ttggtacctt tatggccaga cattgctgat |
| 5401 | attgataatg ctatacatcc gggcattatg tctatattga acagcttggc tgaattgggt |
| 5461 | gacatgatta agttagaagg tggtaattgg ctcacagctc ccccacatgc agtacgaatt |
| 5521 | gacaataaga tggctgtttt ttttggtgga gagccttcct gtacattttc aacgggcgtg |
| 5581 | gtagctaaat ctgctggaag agttcgcttg gttgaagaaa aagtgtgtac tggaagtgtt |
| 5641 | gaaatctggg atgcaaatga gtggattggt gccccagcag aaggcaatga agaatggtca |
| 5701 | tccagactac tatctggaac tatttccggc tttatcgatg cgcctagcaa tatgagtgaa |
| 5761 | acgactgcat atgtgcgggg aaaatggctc catttgtcag aactttcttt taataaaaag |
| 5821 | caaatctact tatgcagaat gtccgttgat aatcactttt cctattattt aggagaaatt |
| 5881 | gaagctggac gcttatgtag aatgaattcg ttagaatcgt ctgatgatgt cagaagatta |
| 5941 | cgtttttttc tcgatacaaa agataattgt ccgctaaaga tccgtatcaa aatatctaat |
| 6001 | gggctagcaa gattaagatt aaccagaaga ttaccaagac gagaaacgaa ggtactcctg |
| 6061 | ctaggctgga gagaatcagg ttttgaaaat gaacattcag gaataacaca ccatgtattc |
| 6121 | cccgaggaaa tattacccat agtgcgtagc gcttttgaag ggcttggtat tatttggatt |
| 6181 | aacgaattca cgcgacggaa tgaaatatga ttaataaaaa taaagtaact gaacgttcag |
| 6241 | gtatacatga taccgtgaaa agccttagtg agaatctgag aaaatacatt gaggcacaat |
| 6301 | atcatatccg ggatgaaggg ttaattgctg agcgacgagc gcttttacag caaaatgaaa |
| 6361 | ctattgctca agctccttat atagaagcaa ccccaattta tgaacctggt gcgccataca |
| 6421 | gtgaattacc tattcccgaa gcagcaagta atgtgctaac tcaactatca gaacttggaa |
| 6481 | ttggcctcta tcaacgcccc tataaacacc aatcacaggc acttgagtca tttcttggcg |
| 6541 | aagacgcttc tgatctggtc attgcaacag gtacaggctc cggtaagact gaaagctttc |
| 6601 | taatgccaat tattggtaaa ttggcgattg aatcttccga gagacctaaa tctgcatccc |
| 6661 | ttccaggttg tagagcaatt ttattatatc caatgaatgc attagttaac gatcaacttg |
| 6721 | ctcgtatcag acgtcttttt ggtgattctg aagcctctaa aatactgaga tctggaagat |
| 6781 | gtgcccctgt acgctttggc gcttatacgg gaagaacgcc ttaccctggt cgtcgtagct |
| 6841 | ctagacgaga cgagcttttt atcaaacccc ttttcgatga gttttacaat aaactcgcaa |
| 6901 | ataacgcccc tgtacgtgcg gaactgaacc gcattggtcg ctggccaagt aaagatcttg |
| 6961 | atgcttttta tgggcaaagc gcatctcagg ctaaaaccta cgtctcaggc aaaaaaacgg |
| 7021 | gtaagcaatt tgttttgaac aattgggggg agaggctaat tacccagcct gaggatcgtg |
| 7081 | agctaatgac ccggcatgaa atgcagaatc gctgtccaga attactgata acgaactact |
| 7141 | ccatgcttga gtatatgctg atgcgaccta tcgagcgtaa tatttttgag cagactaagg |
| 7201 | aatggctcaa agctgatgag atgaatgagc ttatcttagt gcttgatgaa gcgcatatgt |
| 7261 | atagaggagc agggggagca gaggtagccc ttttaatacg tcgcctctgt gctcggttgg |
| 7321 | atattccccg ggaacgtatg cgctgcatcc ttaccagtgc tagtctaggg tccattgagg |
| 7381 | atggagaacg ttttgcccaa gacttaactg gcttatcacc aacctcttcg aggaaatttc |
| 7441 | gaattattga gggtacaagg gaatcgcgtc ctgagtcaca aattgttacc agtaaagaag |
| 7501 | ctaatgcact ggctgaattc gacctaaatt catttcagtg cgtagctgag gatcttgaat |
| 7561 | ctgcatatgc agcaatagag tctcttgccg aacgaatggg ctggcaaaag ccgatgataa |
| 7621 | aagatcatag tacactacgt aattggttat ttgataattt gactggtttt ggtcctattg |
| 7681 | aaacgcttat tgaaatagtt tcaggtaaag cggttaagct aaatatcttg agtgaaaacc |
| 7741 | tttttccaga ctctccacag caaattgcag agcgagcgac agatgcatta ctcgcattgg |
| 7801 | gttgctatgc tcagagggca tccgatggca gagtgcttat tccaactcgc atgcatcttt |
| 7861 | tttatcgggg attaccaggt ctttatgcct gtatagatcc cgattgtaat caacgtttgg |
| 7921 | gtaaccatag tgggccaact atacttggcc gcctttatac gaaaccactg gatcaatgta |
| 7981 | aatgcgcttc aaaagggcga gtctacgaat tatttaccca ccgtgactgc ggtgcggctt |
| 8041 | ttattcgtgg atacgttagt tccgaaatgg actttgtatg gcaccagccg aacggaccat |
| 8101 | tatcagaaga tgaggatatc gatcttgttc ccatagatat attggtcgag gaaacacctc |
| 8161 | atgtacatag tgattaccag gacagatggc tacatatagc aacaggacgc ctttctaaac |
| 8221 | agtgtcaaga tgaggattct ggttatcgta aagtctttat acctgaccga gttaagtctg |
| 8281 | gatctgaaat tacatttgat gaatgccctg tttgtatgcg taagacaaga agtgctcaga |
| 8341 | atgaaccgtc taaaattatg gatcatgtta caaaagggga agcacctttt acaacgttag |
| 8401 | tacgtacaca gatatctcac cagccagcga gtcgtcctat tgatggtaaa catcccaatg |
| 8461 | ggggaaaaaa agtacttatt ttttctgatg gccgacaaaa agcagctcgg cttgcacgtg |
| 8521 | atattcctag agatattgag cttgatttgt ttcggcaatc cattgctctc gcctgttcta |
| 8581 | aactgaaaga tatcaatcgg gaacccaaac caacatcagt actttacctt gctttcctat |
| 8641 | cagtcctttc tgaacatgac ttgcttattt ttgatgggga agattcacga aaagttgtaa |
| 8701 | tggcccgtga tgaattttat cgtgattata atagcgatct ggctcaagct tttgatgata |
| 8761 | acttcagtcc ccaagagtca ccgtcacgat ataaaatagc gttgcttaaa cttttatgta |
| 8821 | gcaattacta ttctctttcc ggaacaacag ttggttttgt tgaaccatcg cagcttaaat |
| 8881 | caaaaaaaat gtgggaagat gtgcagtcca agaagctaaa tattgagagc aaggatgttc |
| 8941 | atgctttagc tgttgcttgg attgatacct tactcactga atttgctttt gatgaatcta |
| 9001 | ttgattcgac actacgaatc aaagccgctg gattctacaa acccacttgg ggtagtcaag |
| 9061 | gacggtttgg aaaagctctt aggaaaaccc tgatacagca tcctgctatg ggggagcttt |
| 9121 | atgtggaagt tttggaggag atttttcgta ctcatctgac attaggaaaa gatggtgtct |
| 9181 | actttcttgc tccaaatgca ctacgtctga aaatagatct cttgcatgtc tggaaacaat |
| 9241 | gtaatgactg cacggcacta atgccatttg ctttagaaca ttctacttgc cttgcttgtg |
| 9301 | gtagtaacag tgtcaaaaca gtcgagccgt cggaaagcag ctatattaat gcacgaaaag |
| 9361 | gattctggcg ttcgccggta gaagaagttt tggtttcaaa ttcgcggctt ctaaacctta |
| 9421 | gcgttgaaga gcatactgct caactctcac atagagatag ggccagcgtt catgccacta |
| 9481 | cagaactcta cgaactgaga ttccaagatg ttcttattaa tgataacgac aagcccattg |
| 9541 | atgtacttag ttgtacgacg acgatggaag tgggggttga tattggatct ctggttgctg |
| 9601 | ttgctttaag aaacgtccct ccgcaacgag aaaattatca gcaacgtgct gggcgagcag |
| 9661 | gccgccgtgg cgcatctgtt tcaacggtgg ttacatattc tcaaaatggc cctcatgata |
| 9721 | gttattattt ccttaatcct gaacgcattg ttgcaggttc tcctcgtaca cctgaagtga |
| 9781 | aagtaaataa tcccaaaata gccagaagac acgttcattc ttttttagtt cagacctttt |
| 9841 | ttcacgagtt aatggaacaa ggaatttata atcccacaga gaaaactgcc atacttgaga |
| 9901 | aagcacttgg tactacacga gatttttttc atggagcaaa agatactggc ctaaatctcg |
| 9961 | atagctttaa taattgggtt aaaaaccgta ttctatctac taatggtgat ttgagaacaa |
| 10021 | gtgttgcagc atggcttcct cctgttcttg aaactggagg gctttctgcc agtgactggt |
| 10081 | ttgctaaggt agcagaggaa tttttaaata cactccatgg gctggctgaa attgttccac |
| 10141 | aaattgccgc tcttgttgat gaggaaaatg aagatgatga gcagacttct ggtggaatga |
| 10201 | aatttgcaca agaagaatta cttgagttcc tgttttacca tggtttatta ccaagttatg |
| 10261 | catttcctac aagcctctgt agtttcttgg tagaaaaaat tgtaaagaat attagaggtt |
| 10321 | cttttgaggt gcgaacagta caacagcctc agcaatcaat ttctcaggct ctgagtgaat |
| 10381 | atgccccggg acgtttgatt gttattgata ggaaaaccta tcgctctggt ggtgtttttt |
| 10441 | ctaatgcatt gaaaggcgaa ctaaaccggg caagaaagct tttcaataat cccaaaaagt |
| 10501 | ttattcattg cgataagtgc tcttttgtcc gcgatcctca taataatcag aatagcgaaa |
| 10561 | atacttgtcc gatctgtggt ggcattctaa aagtagaaat aatgattcag cccgaagtct |
| 10621 | ttggacctga aaatgccaag gaacttaatg aggacgacag agagcaagaa atcacctatg |
| 10681 | taactgcggc acaatatcca caacctgttg atcctgaaga ttttaagttc aataatggag |
| 10741 | gtgctcatat tgtttttact cacgcaatag atcagaaact ggtgacggtg aaccgaggga |
| 10801 | aaaatgaggg ggggtccagt ggtttttcag tatgttgcga atgtggtgcg gcctccgttt |
| 10861 | atgattccta ctcaccggca aagggggcac atgaaagacc gtataaatat atagcaacta |
| 10921 | aggaaacgcc tcgcttatgc tctggcgagt ataaacgcgt ttttctcgga catgatttcc |
| 10981 | gtactgattt gcttttatta cgaataaccg ttgggtctcc gcttgtaact gatacttcaa |
| 11041 | atgctatcgt tttacggatg tatgaagatg cattatatac aatagcggaa gcactaaggc |
| 11101 | ttgcagctag tcgccataaa caactggatc ttgatcctgc tgagtttggc tctggtttca |
| 11161 | gaattttacc cactatagag gaagatactc aggcattgga tctcttcctt tatgatactt |
| 11221 | tatctggcgg tgcgggttat gcggaagtag cagcagcgaa tctagatgac attcttactg |
| 11281 | caacactcgc attgttagaa ggttgtgagt gcgatacctc ctgtacagat tgtctcaatc |
| 11341 | atttccacaa ccagcatata caaagccgtc tcgataggaa actaggtgca tctttacttc |
| 11401 | gttatgctct atacggaatg gttcctcgtt gtgcttcacc tgatattcag gtagaaaaat |
| 11461 | tgtctcaatt gagggcaagt ctggaattgg atggttttca atgcctaatt aagggaactc |
| 11521 | aggaggcacc tatgattgtg agtttgaatg accgttctgt tgcagtggga agttaccctg |
| 11581 | gtcttattga tcgacccgac tttcaacacg acgtatataa gtcaaagaat actaatgctc |
| 11641 | atatagcctt taatgaatat cttcttcgtt caaatctgcc acaagcgcat caaaatatta |
| 11701 | gaaaattgtt gcgctgatag cagtattgag tgccctaaag tcctataggg cactcaaggt |
| 11761 | tattctcttt taatatttgt taatagcatg tattttaagg ctttcagtcc ttgagctggc |
| 11821 | ttttacacaa cgagaaagag gtaattataa taccagccgg atatggcgag aaatggtggc |
| 11881 | ccgggggtgt aagtatcccg cataatcgtg ccattcatat ttagagatca tccggcataa |
| 11941 | tcaatctgct aacgaaggag atcgcta. |

The coding regions for each of the druA, drub, druC, drub, and druE gene sequences within this embodiment of a Druantia Type I cassette (SEQ ID NO: 16) are as follows: nucleotides 378-1592 encode an embodiment of a DruA polypeptide; nucleotides 16614-3260 encode an embodiment of a DruB polypeptide; nucleotides 3272-5155 encode an embodiment of a DruC polypeptide, nucleotides 5167-6210 encode an embodiment of a DruD polypeptide; and nucleotides 6207-11717 encode an embodiment of a DruE polypeptide.

In some embodiments, a Defense System IIIa comprising a DruA polypeptide, a DruB polypeptide, a DruC polypeptide, a DruD polypeptide and a DruE polypeptide is encoded by the nucleic acid sequence set forth in SEQ ID NO: 16. In some embodiments, a Defense System IIIa comprising a DruA polypeptide, a DruB polypeptide, a DruC polypeptide, a DruD polypeptide and a DruE polypeptide, is encoded by a nucleic acid sequence having at least 80% homology to the sequence set forth in SEQ ID NO: 16. In some embodiments, a Defense System IIIa comprising a DruA polypeptide, a DruB polypeptide, a DruC polypeptide, a DruD polypeptide and a DruE polypeptide, is encoded by a nucleic acid sequence having at least 85% homology, or 90% homology, or 95% homology, or 97% homology, or 98% homology, or 99% homology to the sequence set forth in SEQ ID NO: 16. In some embodiments, a Defense System IIIa comprising a DruA polypeptide, a DruB polypeptide, a DruC polypeptide, a DruD polypeptide and a DruE polypeptide, is encoded by a nucleic acid sequence having at least 80% identity to the sequence set forth in SEQ ID NO: 16. In some embodiments, a Defense System IIIa comprising a DruA polypeptide, a DruB polypeptide, a DruC polypeptide, a DruD polypeptide and a DruE polypeptide, is encoded by a nucleic acid sequence having at least 85% identity, or 90% identity, or 95% identity, or 97% identity, or 98% identity, or 99% identity to the sequence set forth in SEQ ID NO: 16.

In some embodiments, the Type I Druantia anti-phage defense system comprises a nucleic acid construct comprising nucleic acid sequences encoding a gene cassette comprising druB, druC, druD, and druE genes. In some embodiments, the Type I Druantia anti-phage defense system comprises a nucleic acid construct comprising nucleic acid sequences encoding a gene cassette comprising druB, druC, druD, druE, and druA genes.

In some embodiments, a construct comprising the Type I Druantia anti-phage defense system encodes one component of the defense system, whereby multiple constructs may be used to assemble the functional defense system. In some embodiments, the components of a Druantia defense system comprise polypeptides DruA, DruB, DruC, DruD, and DruE. In some embodiments, the components of a Druantia Type I defense system comprise polypeptides DruB, DruC, DruD, and DruE.

In some embodiments, a construct comprising the Druantia Type I anti-phage defense system encodes one component of the defense system, whereby multiple constructs may be used to assemble the functional defense system. In some embodiments, the components of a Druantia defense system comprise genes druA, druB, druC, druD, and druE. In some embodiments, the components of a Druantia Type I defense system comprise genes druB, druC, druD, and druE.

In some embodiments, the components of a Druantia Type I defense system consists of genes druA, druB, druC, druD, and druE. In some embodiments, the components of a Druantia Type I defense system consist of genes druB, druC, druD, and druE.

In some embodiments, the components of a Druantia Type I defense system comprise nucleic acid sequences encoding a DruA polypeptide, a DruB polypeptide, a DruC polypeptide, a DruD polypeptide, and a DruE polypeptide. In some embodiments, the components of a Druantia Type I defense system comprise nucleic acid sequences encoding a DruB polypeptide, a DruC polypeptide, a DruD polypeptide, and a DruE polypeptide. In some embodiments, the components of a Druantia Type I defense system comprise nucleic acid sequences encoding a DruB polypeptide, a DruC polypeptide, a DruD polypeptide, a DruE polypeptide, and a DruA polypeptide.

In some embodiments, the components of a Druantia Type I defense system consist of nucleic acid sequences encoding a DruA polypeptide, a DruB polypeptide, a DruC polypeptide, a DruD polypeptide, and a DruE polypeptide. In some embodiments, the components of a Druantia Type I defense system consist of nucleic acid sequences encoding a DruB polypeptide, a DruC polypeptide, a DruD polypeptide, and a DruE polypeptide. In some embodiments, the components of a Druantia Type I defense system consist of nucleic acid sequences encoding a DruB polypeptide, a DruC polypeptide, a DruD polypeptide, a DruE polypeptide, and a DruA polypeptide.

In some embodiments, a construct comprising the Druantia Type I defense system encodes one component of the defense system, whereby a second, third, fourth, or fifth construct may be used to assemble the functional defense system. In some embodiments, a construct comprising the Druantia defense system encodes one component of the defense system, whereby additional constructs may be used to assemble the functional defense system. For example, in some embodiments each of DruA, DruB, DruC, DruD, and DruE may be encoded by nucleic acid sequences comprised in different constructs, wherein expression of the combination of constructs produces a functional Druantia defense system. In some embodiments each of DruB, DruC, DruD, and DruE may be encoded by nucleic acid sequences comprised in different constructs, wherein expression of the combination of constructs produces a functional Druantia defense system.

In some embodiments, the components making up a functional Druantia Type I anti-phage defense system comprise a DruA polypeptide, a DruB polypeptide, a DruC polypeptide, a DruD polypeptide, and a DruE polypeptide, each encoded by a druA, druB, druC, druD, or druE gene, respectively. In some embodiments, the components making up a functional Druantia Type I anti-phage defense system comprise a DruB polypeptide, a DruC polypeptide, a DruD polypeptide, and a DruE polypeptide, each encoded by a druB, druC, druD, or druE gene, respectively.

In some embodiments, a Druantia Type I defense system having an anti-phage activity comprises a nucleic acid encoding a DruA polypeptide. In some embodiments, a Druantia Type I defense system having an anti-phage activity comprises a nucleic acid encoding a DruB polypeptide. In some embodiments, a Druantia Type I defense system having an anti-phage activity comprises a nucleic acid encoding a DruC polypeptide. In some embodiments, a Druantia Type I defense system having an anti-phage activity comprises a nucleic acid encoding a DruD polypeptide. In some embodiments, a Druantia Type I defense system having an anti-phage activity comprises a nucleic acid encoding a DruE polypeptide.

In some embodiments, a Druantia Type I defense system having an anti-phage activity comprises a nucleic acid encoding a DruB polypeptide and a DruE polypeptide. In some embodiments, a Druantia defense system having an anti-phage activity comprises a nucleic acid encoding a DruC polypeptide and a DruE polypeptide. In some embodiments, a Druantia Type I defense system having an anti-phage activity comprises a nucleic acid encoding a DruB polypeptide, a DruC polypeptide, and a DruE polypeptide.

In some embodiments, a Druantia Type I defense system having an anti-phage activity comprise a nucleic acid comprising a druA gene. In some embodiments, a Druantia Type I defense system having an anti-phage activity comprise a nucleic acid comprising a druB gene. In some embodiments, a Druantia Type I defense system having an anti-phage activity comprise a nucleic acid comprising a druC gene. In some embodiments, a Druantia Type I defense system having an anti-phage activity comprise a nucleic acid comprising a druD gene. In some embodiments, a Druantia Type I defense system having an anti-phage activity comprise a nucleic acid comprising a druE gene.

In some embodiments, a Druantia Type I defense system having an anti-phage activity comprises a nucleic acid construct comprising nucleic acids encoding polypeptides in a set order. In one embodiment, the 5′ to 3′ order of polypeptides encoded is DruA, DruB, DruC, DruD, and DruE. In one embodiment, the 5′ to 3′ order of polypeptides encoded is DruB, DruC, DruD, and DruE. In one embodiment, the 5′ to 3′ order of polypeptides encoded is DruB, DruC, DruD, DruE, and DruA.

In some embodiments, the 5′ to 3′ order of polypeptides is random, for example any order of DruA, DruB, DruC, DruD, and DruE. In some embodiments, the 5′ to 3′ order of polypeptides is random, for example any order of DruB, DruC, DruD, and DruE.

In some embodiments the 5′ to 3′ order of polypeptides does not affect the anti-phage activity. In some embodiments the 5′ to 3′ order of polypeptides does affect the anti-phage activity.

In some embodiments, a Druantia Type I defense system having an anti-phage activity comprises a nucleic acid construct comprising genes in a set order. In some embodiment, the 5′ to 3′ order of genes is druA, druB, druC, druD, and druE. In some embodiment, the 5′ to 3′ order of genes is druB, druC, druD, and druE. In some embodiment, the 5′ to 3′ order of genes is druB, druC, druD, druE, and druA.

In some embodiments, the 5′ to 3′ order of genes is random, for example any order of druA, druB, druC, druD, and druE. In some embodiments, the 5′ to 3′ order of genes is random, for example any order of druB, druC, druD, and druE.

In some embodiments the 5′ to 3′ order of genes does not affect the anti-phage activity. In some embodiments the 5′ to 3′ order of genes does affect the anti-phage activity.

In some embodiments, a Druantia Type II defense system (Defense System IIIb) having an anti-phage activity comprises a nucleic acid construct comprising a nucleic acid sequence encoding a DruM polypeptide comprising a COG0270 domain or a pfam00145 domain or a combination thereof, a DruF polypeptide, a DruG polypeptide, and a DruE polypeptide comprising a pfam00270 domain or a pfam00271 domain or a pfam09369 domain or a DUF1998 domain or any combination thereof. In some embodiments, a Druantia Type II defense system (Defense System IIIb) having an anti-phage activity comprises a nucleic acid construct comprising a nucleic acid sequence encoding a DruM polypeptide comprising a COG0270 domain or a pfam00145 domain or a combination thereof, a DruF polypeptide wherein said DruF polypeptide is encoded by a gene positioned within 5 genes of a gene encoding said DruM polypeptide and said DruE polypeptide, a DruG polypeptide wherein said DruG polypeptide is encoded by a gene positioned within 5 genes of a gene encoding said DruM polypeptide and said DruE polypeptide, and a DruE polypeptide comprising a pfam00270 domain or a pfam00271 domain or a pfam09369 domain or a DUF1998 domain or any combination thereof.

In some embodiments, a Druantia Type II defense system (Defense System IIIb) having an anti-phage activity comprises a DruM polypeptide comprising a COG0270 domain or a pfam00145 domain or a combination thereof, a DruF polypeptide, a DruG polypeptide, and a DruE polypeptide comprising a pfam00270 domain or a pfam00271 domain or a pfam09369 domain or a DUF1998 domain or any combination thereof. In some embodiments, a Druantia Type II defense system (Defense System IIIb) having an anti-phage activity comprises a DruM polypeptide comprising a COG0270 domain or a pfam00145 domain or a combination thereof, a DruF polypeptide wherein said DruF polypeptide is encoded by a gene positioned within 5 genes of a gene encoding said DruM polypeptide and said DruE polypeptide, a DruG polypeptide wherein said DruG polypeptide is encoded by a gene positioned within 5 genes of a gene encoding said DruM polypeptide and said DruE polypeptide, and a DruE polypeptide comprising a pfam00270 domain or a pfam00271 domain or a pfam09369 domain or a DUF1998 domain or any combination thereof.

In some embodiments, a Defense System IIIb comprises a DruM polypeptide comprising a pfam00145 domain or a COG0270 domain or a combination thereof, or comprising an amino acid sequence having at least 80% identity to a sequence selected from the group referenced in Table 10 rows 124-295 columns AD and AE; or a DruF polypeptide comprising an amino acid sequence having at least 80% identity to a sequence selected from the group referenced in Table 10 rows 124-295 columns AH and AI; or a DruG polypeptide comprising an amino acid sequence having at least 80% identity to a sequence selected from the group referenced in Table 10 rows 124-295 columns AL and AM; or a DruE polypeptide comprising a pfam00270 domain or a pfam00271 domain or a pfam09369 domain or a DUF1998 domain or a combination thereof, or comprising an amino acid sequence having at least 80% identity to a sequence selected from the group referenced in Table 10 rows 2-1343 columns Z and AA; or any combination thereof.

In some embodiments a Defense System IIIb comprises at least two different polypeptide components selected from a DruM polypeptide comprising a pfam00145 domain or a COG0270 domain or a combination thereof, or comprising an amino acid sequence having at least 80% identity to a sequence selected from the group referenced in Table 10 rows 124-295 columns AD and AE; or a DruF polypeptide comprising an amino acid sequence having at least 80% identity to a sequence selected from the group referenced in Table 10 rows 124-295 columns AH and AI; or a DruG polypeptide comprising an amino acid sequence having at least 80% identity to a sequence selected from the group referenced in Table 10 rows 124-295 columns AL and AM; or a DruE polypeptide comprising a pfam00270 domain or a pfam00271 domain or a pfam09369 domain or a DUF1998 domain or a combination thereof, or comprising an amino acid sequence having at least 80% identity to a sequence selected from the group referenced in Table 10 rows 2-1343 columns Z and AA; or any combination thereof. In some embodiments a Defense System IIIb comprises at least three different polypeptide components selected from a DruM polypeptide comprising a pfam00145 domain or a COG0270 domain or a combination thereof, or comprising an amino acid sequence having at least 80% identity to a sequence selected from the group referenced in Table 10 rows 124-295 columns AD and AE; or a DruF polypeptide comprising an amino acid sequence having at least 80% identity to a sequence selected from the group referenced in Table 10 rows 124-295 columns AH and AI; or a DruG polypeptide comprising an amino acid sequence having at least 80% identity to a sequence selected from the group referenced in Table 10 rows 124-295 columns AL and AM; or a DruE polypeptide comprising a pfam00270 domain or a pfam00271 domain or a pfam09369 domain or a DUF1998 domain or a combination thereof, or comprising an amino acid sequence having at least 80% identity to a sequence selected from the group referenced in Table 10 rows 2-1343 columns Z and AA; or any combination thereof. In some embodiments a Defense System IIIb comprises four different polypeptide components selected from a DruM polypeptide comprising a pfam00145 domain or a COG0270 domain or a combination thereof, or comprising an amino acid sequence having at least 80% identity to a sequence selected from the group referenced in Table 10 rows 124-295 columns AD and AE; or a DruF polypeptide comprising an amino acid sequence having at least 80% identity to a sequence selected from the group referenced in Table 10 rows 124-295 columns AH and AI; or a DruG polypeptide comprising an amino acid sequence having at least 80% identity to a sequence selected from the group referenced in Table 10 rows 124-295 columns AL and AM; or a DruE polypeptide comprising a pfam00270 domain or a pfam00271 domain or a pfam09369 domain or a DUF1998 domain or a combination thereof, or comprising an amino acid sequence having at least 80% identity to a sequence selected from the group referenced in Table 10 rows 2-1343 columns Z and AA; or any combination thereof.

In some embodiments, a functional Defense System IIIb comprises at least two different polypeptide components selected from a DruM polypeptide comprising a pfam00145 domain or a COG0270 domain or a combination thereof, or comprising an amino acid sequence having at least 80% identity to a sequence selected from the group referenced in Table 10 rows 124-295 columns AD and AE; or a DruF polypeptide comprising an amino acid sequence having at least 80% identity to a sequence selected from the group referenced in Table 10 rows 124-295 columns AH and AI; or a DruG polypeptide comprising an amino acid sequence having at least 80% identity to a sequence selected from the group referenced in Table 10 rows 124-295 columns AL and AM; or a DruE polypeptide comprising a pfam00270 domain or a pfam00271 domain or a pfam09369 domain or a DUF1998 domain or a combination thereof, or comprising an amino acid sequence having at least 80% identity to a sequence selected from the group referenced in Table 10 rows 2-1343 columns Z and AA; or any combination thereof. In some embodiments, a functional Defense System IIIb comprises at least three different polypeptide components selected from a DruM polypeptide comprising a pfam00145 domain or a COG0270 domain or a combination thereof, or comprising an amino acid sequence having at least 80% identity to a sequence selected from the group referenced in Table 10 rows 124-295 columns AD and AE; or a DruF polypeptide comprising an amino acid sequence having at least 80% identity to a sequence selected from the group referenced in Table 10 rows 124-295 columns AH and AI; or a DruG polypeptide comprising an amino acid sequence having at least 80% identity to a sequence selected from the group referenced in Table 10 rows 124-295 columns AL and AM; or a DruE polypeptide comprising a pfam00270 domain or a pfam00271 domain or a pfam09369 domain or a DUF1998 domain or a combination thereof, or comprising an amino acid sequence having at least 80% identity to a sequence selected from the group referenced in Table 10 rows 2-1343 columns Z and AA; or any combination thereof. In some embodiments, a functional Defense System IIIb comprises four different polypeptide components selected from a DruM polypeptide comprising a pfam00145 domain or a COG0270 domain or a combination thereof, or comprising an amino acid sequence having at least 80% identity to a sequence selected from the group referenced in Table 10 rows 124-295 columns AD and AE; or a DruF polypeptide comprising an amino acid sequence having at least 80% identity to a sequence selected from the group referenced in Table 10 rows 124-295 columns AH and AI; or a DruG polypeptide comprising an amino acid sequence having at least 80% identity to a sequence selected from the group referenced in Table 10 rows 124-295 columns AL and AM; or a DruE polypeptide comprising a pfam00270 domain or a pfam00271 domain or a pfam09369 domain or a DUF1998 domain or a combination thereof, or comprising an amino acid sequence having at least 80% identity to a sequence selected from the group referenced in Table 10 rows 2-1343 columns Z and AA; or any combination thereof.

In some embodiments, a Druantia Type II defense system (Defense System IIIb) having an anti-phage activity comprises a nucleic acid construct comprising a nucleic acid sequence comprising a druM gene, a druF gene, a druG gene, and a druE gene.

In some embodiments, the Druantia Type II anti-phage defense system comprises a nucleic acid construct comprising nucleic acid sequences encoding a gene cassette comprising druM, druF, druG, and druE genes. In some embodiments, the Type II Druantia anti-phage defense system comprises a nucleic acid construct comprising nucleic acid sequences encoding a gene cassette comprising druM, druF, druG, and druE genes.

In some embodiments, a construct comprising the Type II Druantia anti-phage defense system encodes one component of the defense system, whereby multiple constructs may be used to assemble the functional defense system. In some embodiments, the components of a Druantia defense system comprise polypeptides DruM, DruF, DruG, and DruE.

In some embodiments, a construct comprising the Druantia Type II anti-phage defense system encodes one component of the defense system, whereby multiple constructs may be used to assemble the functional defense system. In some embodiments, the components of a Druantia defense system comprise genes druM, druF, druG, and druE.

In some embodiments, the components of a Druantia Type II defense system consists of genes druM, druF, druG, and druE.

In some embodiments, the components of a Druantia Type II defense system comprise nucleic acid sequences encoding a DruM polypeptide, a DruF polypeptide, a DruG polypeptide, and a DruE polypeptide.

In some embodiments, the components of a Druantia Type II defense system consist of nucleic acid sequences encoding a DruM polypeptide, a DruF polypeptide, a DruG polypeptide, and a DruE polypeptide.

In some embodiments, a construct comprising the Druantia Type II defense system encodes one component of the defense system, whereby a second, third, or fourth, construct may be used to assemble the functional defense system. In some embodiments, a construct comprising the Druantia defense system encodes one component of the defense system, whereby additional constructs may be used to assemble the functional defense system. For example, in some embodiments each of DruM, DruF, DruG, and DruE may be encoded by nucleic acid sequences comprised in different constructs, wherein expression of the combination of constructs produces a functional Druantia Type II defense system.

In some embodiments, the components making up a functional Druantia Type II anti-phage defense system comprise a DruM polypeptide, a DruF polypeptide, a DruG polypeptide, and a DruE polypeptide, each encoded by a druM, druF, druG, or druE gene, respectively.

In some embodiments, a Druantia Type II defense system having an anti-phage activity comprises a nucleic acid encoding a DruM polypeptide. In some embodiments, a Druantia Type II defense system having an anti-phage activity comprises a nucleic acid encoding a DruF polypeptide. In some embodiments, a Druantia Type II defense system having an anti-phage activity comprises a nucleic acid encoding a DruG polypeptide. In some embodiments, a Druantia Type II defense system having an anti-phage activity comprises a nucleic acid encoding a DruE polypeptide.

In some embodiments, a Druantia Type II defense system having an anti-phage activity comprises a nucleic acid encoding a DruM polypeptide and a DruE polypeptide. In some embodiments, a Druantia defense system having an anti-phage activity comprises a nucleic acid encoding a DruF polypeptide and a DruE polypeptide. In some embodiments, a Druantia Type II defense system having an anti-phage activity comprises a nucleic acid encoding a DruG polypeptide and a DruE polypeptide. In some embodiments, a Druantia defense system having an anti-phage activity comprises a nucleic acid encoding a DruM polypeptide, a DruF polypeptide, and a DruE polypeptide. In some embodiments, a Druantia Type II defense system having an anti-phage activity comprises a nucleic acid encoding a DruM polypeptide, a DruG polypeptide, and a DruE polypeptide. In some embodiments, a Druantia Type II defense system having an anti-phage activity comprises a nucleic acid encoding a DruM polypeptide, a DruF polypeptide, and a DruG polypeptide. In some embodiments, a Druantia Type II defense system having an anti-phage activity comprises a nucleic acid encoding a DruF polypeptide, a DruG polypeptide, and a DruE polypeptide. In some embodiments, a Druantia Type II defense system having an anti-phage activity comprises a nucleic acid encoding a DruF polypeptide and a DruG polypeptide.

In some embodiments, a Druantia Type II defense system having an anti-phage activity comprise a nucleic acid comprising a druM gene. In some embodiments, a Druantia Type II defense system having an anti-phage activity comprise a nucleic acid comprising a druF gene. In some embodiments, a Druantia Type II defense system having an anti-phage activity comprise a nucleic acid comprising a druG gene. In some embodiments, a Druantia Type II defense system having an anti-phage activity comprise a nucleic acid comprising a druE gene.

In some embodiments, a Druantia Type II defense system having an anti-phage activity comprises a nucleic acid construct comprising nucleic acids encoding polypeptides in a set order. In one embodiment, the 5′ to 3′ order of polypeptides encoded is DruM, DruF, DruG, and DruE.

In some embodiments, the 5′ to 3′ order of polypeptides is random, for example any order of DruM, DruF, DruG, and DruE.

In some embodiments the 5′ to 3′ order of polypeptides does not affect the anti-phage activity. In some embodiments the 5′ to 3′ order of polypeptides does affect the anti-phage activity.

In some embodiments, a Druantia Type II defense system having an anti-phage activity comprises a nucleic acid construct comprising genes in a set order. Non-limiting examples of the 5′ to 3′ order or genes comprises druM, druF, druG, and druE; druF, druG, druE, and drum; druG, druE, druM, and druF; and druE, druM, druG, and druF.

In some embodiments, the 5′ to 3′ order of genes is random, for example any order of druM, druF, druG, and druE.

In some embodiments the 5′ to 3′ order of genes does not affect the anti-phage activity. In some embodiments the 5′ to 3′ order of genes does affect the anti-phage activity.

In some embodiments, a Druantia Type III defense system (Defense System IIIc) having an anti-phage activity comprises a nucleic acid construct comprising a nucleic acid sequence encoding a DruH polypeptide and a DruE polypeptide comprising a pfam00270 domain or a pfam00271 domain or a pfam09369 domain or a DUF1998 domain or any combination thereof. In some embodiments, a Druantia Type III defense system (Defense System IIIc) having an anti-phage activity comprises a nucleic acid construct comprising a nucleic acid sequence encoding a DruH polypeptide and said DruE polypeptide, wherein said DruH polypeptide is encoded by a gene positioned within 5 genes of a gene encoding said DruE polypeptide.

In some embodiments, a Druantia Type III defense system (Defense System IIIc) having an anti-phage activity comprises a DruH polypeptide and a DruE polypeptide comprising a pfam00270 domain or a pfam00271 domain or a pfam09369 domain or a DUF1998 domain or any combination thereof. In some embodiments, a Druantia Type III defense system (Defense System IIIc) having an anti-phage activity comprises a DruH polypeptide and a DruE polypeptide comprising a pfam00270 domain or a pfam00271 domain or a pfam09369 domain or a DUF1998 domain or any combination thereof, wherein said DruH polypeptide is encoded by a gene positioned within 5 genes of a gene encoding said DruE polypeptide.

In some embodiments, a Defense System IIIc comprises a DruH polypeptide comprising an amino acid sequence having at least 80% identity to a sequence selected from the group referenced in Table 10 rows 296-1343 columns AP and AQ; or a DruE polypeptide comprising a pfam00270 domain or a pfam00271 domain or a pfam09369 domain or a DUF1998 domain or comprising an amino acid sequence having at least 80% identity to a sequence selected from the group referenced in Table 10 rows 2-1343 columns Z nd AA or a combination thereof; or any combination thereof.

In some embodiments, a Druantia Type IIII defense system (Defense System IIIc) having an anti-phage activity comprises a nucleic acid construct comprising a nucleic acid sequence comprising a druH gene and a druE gene.

In some embodiments, the Druantia Type IIII anti-phage defense system comprises a nucleic acid construct comprising nucleic acid sequences encoding a gene cassette comprising druH and druE genes.

In some embodiments, a construct comprising the Type III Druantia anti-phage defense system encodes one component of the defense system, whereby multiple constructs may be used to assemble the functional defense system. In some embodiments, the components of a Druantia defense system comprise polypeptides DruH and DruE.

In some embodiments, a construct comprising the Druantia Type III anti-phage defense system encodes one component of the defense system, whereby multiple constructs may be used to assemble the functional defense system. In some embodiments, the components of a Druantia defense system comprise genes druH and druE.

In some embodiments, the components of a Druantia Type III defense system consists of genes druH and druE.

In some embodiments, the components of a Druantia Type III defense system comprise nucleic acid sequences encoding a DruH polypeptide and a DruE polypeptide.

In some embodiments, the components of a Druantia Type III defense system consist of nucleic acid sequences encoding a DruH polypeptide and a DruE polypeptide.

In some embodiments, a construct comprising the Druantia Type III defense system encodes one component of the defense system, whereby a second construct may be used to assemble the functional defense system. In some embodiments, a construct comprising the Druantia Type III defense system encodes one component of the defense system, whereby an additional construct may be used to assemble the functional defense system. For example, in some embodiments each of DruH and DruE may be encoded by nucleic acid sequences comprised in different constructs, wherein expression of the combination of constructs produces a functional Druantia defense system.

In some embodiments, the components making up a functional Druantia Type III anti-phage defense system (Defense System IIIc) comprise a DruH polypeptide and a DruE polypeptide, each encoded by a druH and druE gene, respectively. In some embodiments, a Defense System IIIc comprises at least two different polypeptide components selected from DruH and DruE, as described herein. In some embodiments, a functional Defense System IIIc comprises at least two different polypeptide components selected from DruH and DruE, as described herein. In some embodiments, a functional Defense System IIIc having anti-phage activity comprises at least two different polypeptide components selected from DruH and DruE, as described herein.

In some embodiments, a Druantia Type III defense system having an anti-phage activity comprises a nucleic acid encoding a DruH polypeptide. In some embodiments, a Druantia Type III defense system having an anti-phage activity comprises a nucleic acid encoding a DruE polypeptide.

In some embodiments, a Druantia Type III defense system having an anti-phage activity comprises a nucleic acid encoding a DruH polypeptide and a DruE polypeptide.

In some embodiments, a Druantia Type III defense system having an anti-phage activity comprise a nucleic acid comprising a druH gene. In some embodiments, a Druantia Type III defense system having an anti-phage activity comprise a nucleic acid comprising a druE gene.

In some embodiments, a Druantia Type III defense system having an anti-phage activity comprises a nucleic acid construct comprising nucleic acids encoding polypeptides in a set order. In one embodiment, the 5′ to 3′ order of polypeptides encoded is DruH and DruE. In one embodiment, the 5′ to 3′ order of polypeptides encoded is DruE and DruH.

In some embodiments, the 5′ to 3′ order of polypeptides is random, for example any order of DruH and DruE.

In some embodiments the 5′ to 3′ order of polypeptides does not affect the anti-phage activity. In some embodiments the 5′ to 3′ order of polypeptides does affect the anti-phage activity.

In some embodiments, a Druantia Type III defense system having an anti-phage activity comprises a nucleic acid construct comprising genes in a set order. In some embodiment, the 5′ to 3′ order of genes is druH and druE.

In some embodiments, the 5′ to 3′ order of genes is random, for example any order of druH and druE.

In some embodiments the 5′ to 3′ order of genes does not affect the anti-phage activity. In some embodiments the 5′ to 3′ order of genes does affect the anti-phage activity.

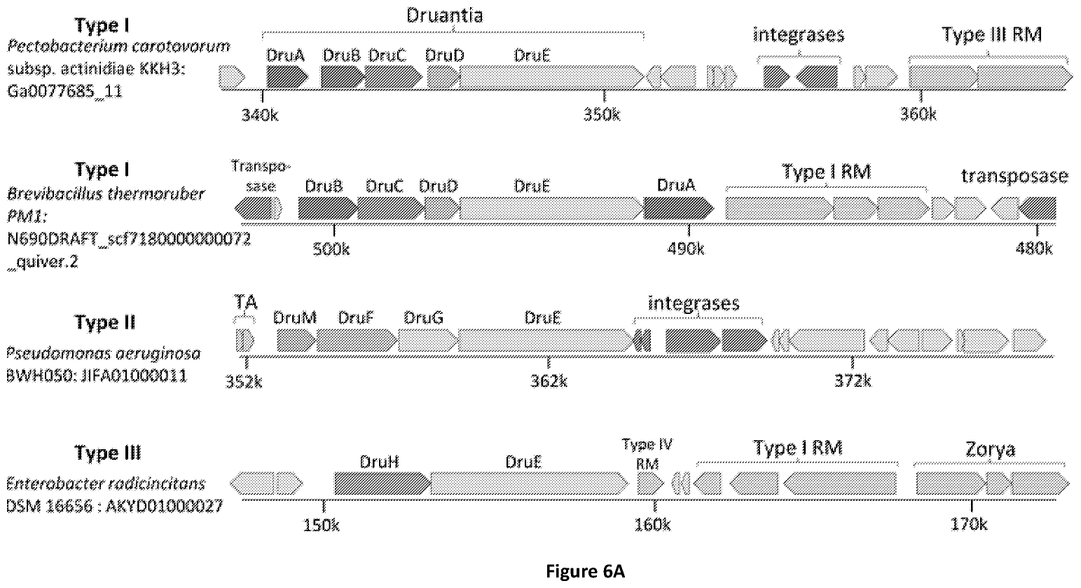

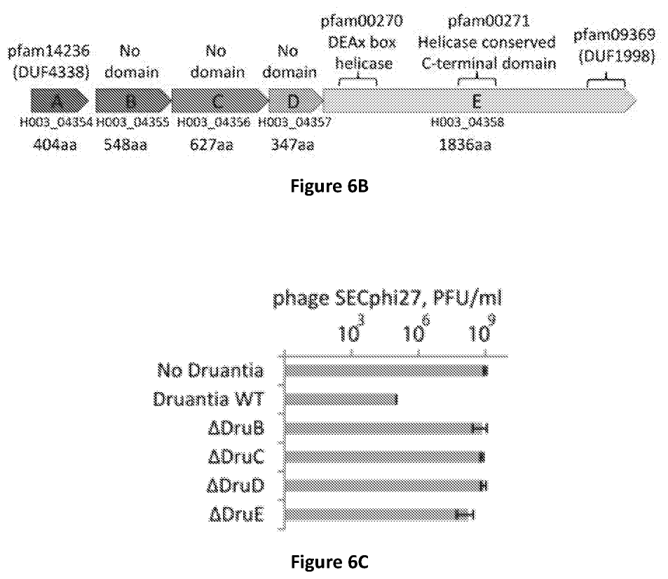

In some embodiments, the Druantia system (Defense Systems IIIa, IIIb, and IIIc) composition and order is as shown in FIG. 3C, FIG. 6A, or FIG. 6B.

In some embodiments, a Druantia defense system having an anti-phage activity originates from a microbial genome, for example a bacterial or an archaeal genome (Table 10). A skilled artisan would appreciate that the Druantia system is not present in the majority of bacteria and or archaea species.

In some embodiments, a functional Druantia defense system (Defense System IIIa, Defense System IIIb, and/or Defense System IIIc) comprises a construct comprising nucleic acid sequences encoding polypeptides, wherein the nucleic acid sequence encoding each polypeptide may originate from a different microbial species. In some embodiments, a functional Druantia defense system (Defense System IIIa, Defense System IIIb, and/or Defense System IIIc) comprises a non-naturally occurring combination of polypeptide components. In some embodiments, a functional Druantia defense system (Defense System IIIa, Defense System IIIb, and/or Defense System IIIc) comprises a combination of at least two polypeptides that do not naturally occur together. In some embodiments, a functional Druantia defense system (Defense System IIIa, Defense System IIIb, and/or Defense System IIIc) comprises a combination of at least three polypeptides that do not naturally occur together. In some embodiments, a functional Druantia defense system (Defense System IIIa, Defense System IIIb, and/or Defense System IIIc) comprises a combination of at least four polypeptides that do not naturally occur together. In some embodiments, a functional Druantia defense system (Defense System IIIa, Defense System IIIb, and/or Defense System IIIc) comprises a combination of at least five polypeptides that do not naturally occur together.

For example, the source of the nucleic acid sequence encoding a DruA polypeptide may be one microbial species, the source of the nucleic acid sequence encoding a DruB polypeptide may be a different microbial species, the source of the nucleic acid sequence encoding a DruC polypeptide may be a different microbial species, the source of the nucleic acid sequence encoding a DruD polypeptide may be a different microbial species, and the source of the nucleic acid sequence encoding a DruE polypeptide may be yet a different microbial species. In some embodiments, the source of the nucleic acid sequence encoding some of the components of the Druantia system Type I (Defense System IIIa; DruA, DruB, DruC, DruD, and DruE) are the same, while the source of others is different. In some embodiments, the source of the nucleic acid sequence encoding the components of the Druantia Type I system (DruA, DruB, DruC, DruD, and DruE) is the same.

In other embodiments, the source of the nucleic acid sequence encoding a DruM polypeptide may be one microbial species, the source of the nucleic acid sequence encoding a DruF polypeptide may be a different microbial species, the source of the nucleic acid sequence encoding a DruG polypeptide may be a different microbial species, and the source of the nucleic acid sequence encoding a DruE polypeptide may be a different microbial species. In some embodiments, the source of the nucleic acid sequence encoding some of the components of the Druantia Type II system (Defense System IIIb; DruM, DruF, DruG, and DruE) are the same, while the source of others is different. In some embodiments, the source of the nucleic acid sequence encoding the components of the Druantia Type II system (DruM, DruF, DruG, and DruE) is the same.

In other embodiments, the source of the nucleic acid sequence encoding a DruH polypeptide may be one microbial species, and the source of the nucleic acid sequence encoding a DruE polypeptide may be a different microbial species. In some embodiments, the source of the nucleic acid sequence encoding the components of the Druantia Type III system (Defense System IIIc; DruH and DruE) is the same.

In some embodiments, a functional Druantia Type I defense system comprises a construct comprising nucleic acid sequences encoding polypeptides, wherein the nucleic acid sequence encoding each polypeptide may originate from a different bacterial species. For example, the source of the nucleic acid sequence encoding a DruA polypeptide may be one bacterial species, the source of the nucleic acid sequence encoding a DruB polypeptide may be a different bacterial species, the source of the nucleic acid sequence encoding a DruC polypeptide may be a different bacterial species, the source of the nucleic acid sequence encoding a DruD polypeptide may be a different bacterial species, and the source of the nucleic acid sequence encoding a DruE polypeptide may be yet a different bacterial species. In some embodiments, the source of the nucleic acid sequence encoding some of the components of the Druantia Type I system (DruA, DruB, DruC, DruD, and DruE) are the same, while the source of others is different. In some embodiments, the source of the nucleic acid sequence encoding the components of the Druantia Type I system (DruA, DruB, DruC, DruD, and DruE) is the same.

In other embodiments, the source of the nucleic acid sequence encoding a DruM polypeptide may be one bacterial species, the source of the nucleic acid sequence encoding a DruF polypeptide may be a different bacterial species, the source of the nucleic acid sequence encoding a DruG polypeptide may be a different bacterial species, and the source of the nucleic acid sequence encoding a DruE polypeptide may be a different bacterial species. In some embodiments, the source of the nucleic acid sequence encoding some of the components of a functional Druantia Type II system (DruM, DruF, DruG, and DruE) are the same, while the source of others is different. In some embodiments, the source of the nucleic acid sequence encoding the components of a functional Druantia Type II system (DruM, DruF, DruG, and DruE) is the same.

In other embodiments, in a functional Type III Druantia system, the source of the nucleic acid sequence encoding a DruH polypeptide may be one bacterial species, and the source of the nucleic acid sequence encoding a DruE polypeptide may be a different bacterial species. In some embodiments, the source of the nucleic acid sequence encoding the components of a functional Druantia Type III system (DruH and DruE) is the same.

In some embodiments, the source of the nucleic acid sequence of any of the components of a Druantia defense system (Defense Systems IIIa [Druantia defense system I], IIIb [Druantia defense system II], and IIIc [Druantia defense system III]) comprises any of the species listed in Table 10.

In some embodiments, a Druantia Type I defense system having an anti-phage activity comprises a nucleic acid construct comprising non-coding regions between the nucleic acid sequences encoding the polypeptides. In some embodiments, the non-coding regions between the nucleic acid sequences comprises nucleic acid sequence not naturally occurring in the microbial species of origin. In some embodiments, the non-coding regions between the nucleic acid sequences comprises nucleic acid sequence is the naturally occurring in the microbial species of origin.

In some embodiments, a Druantia Type II defense system having an anti-phage activity comprises a nucleic acid construct comprising non-coding regions between the nucleic acid sequences encoding the polypeptides. In some embodiments, the non-coding regions between the nucleic acid sequences comprises nucleic acid sequence not naturally occurring in the microbial species of origin. In some embodiments, the non-coding regions between the nucleic acid sequences comprises nucleic acid sequence is the naturally occurring in the microbial species of origin.

In some embodiments, a Druantia Type III defense system having an anti-phage activity comprises a nucleic acid construct comprising non-coding regions between the nucleic acid sequences encoding the polypeptides. In some embodiments, the non-coding regions between the nucleic acid sequences comprises nucleic acid sequence not naturally occurring in the microbial species of origin. In some embodiments, the non-coding regions between the nucleic acid sequences comprises nucleic acid sequence is the naturally occurring in the microbial species of origin.

In some embodiments, a Druantia Type I system disclosed herein comprises a multi-gene phage resistance system broadly distributed in microbial genomes, for example but not limited to bacteria and archaea. Yet, the majority of bacterial species do not express a Druantia Type I defense system.

In some embodiments, a Druantia Type II system disclosed herein comprises a multi-gene phage resistance system broadly distributed in microbial genomes, for example but not limited to bacteria and archaea. Yet, the majority of bacterial species do not express a Druantia Type II defense system.

In some embodiments, a Druantia Type III system disclosed herein comprises a multi-gene phage resistance system broadly distributed in microbial genomes, for example but not limited to bacteria and archaea. Yet, the majority of bacterial species do not express a Druantia Type III defense system.

According to some embodiments, the Druantia systems' (Type I) components are located in a gene cluster (a cassette of genes) in a microbial cell genome. According to some embodiments, the Druantia system components are located in close proximity (e.g. positioned within 20 genes, 10 genes, 5 genes or less one from the other) in a genome of a prokaryotic cell. According to some embodiments the prokaryotic cell expresses an endogenous Druantia Type I defense system. According to some embodiments, the prokaryotic cell expresses an endogenous functional Druantia Type I defense system. According to some embodiments, the species of prokaryotic cell is selected from the group consisting of the species listed in Table 10.

According to some embodiments a prokaryotic cell expresses a non-endogenous Druantia Type I defense system. According to some embodiments, a prokaryotic cell expresses a non-endogenous functional Druantia Type I defense system. According to some embodiments, the species of prokaryotic cell expressing a non-endogenous functional Druantia Type I defense system is selected from the group consisting of the species listed in Table 10.

According to some embodiments, the Druantia systems' (Type II) components are located in a gene cluster (a cassette of genes) in a microbial cell genome. According to some embodiments, the Druantia system components are located in close proximity (e.g. positioned within 20 genes, 10 genes, 5 genes or less one from the other) in a genome of a prokaryotic cell. According to some embodiments the prokaryotic cell expresses an endogenous Druantia Type II defense system. According to some embodiments, the prokaryotic cell expresses an endogenous functional Druantia Type II defense system. According to some embodiments, the species of prokaryotic cell is selected from the group consisting of the species listed in Table 10.

According to some embodiments a prokaryotic cell expresses a non-endogenous Druantia Type II defense system. According to some embodiments, a prokaryotic cell expresses a non-endogenous functional Druantia Type II defense system. According to some embodiments, the species of prokaryotic cell expressing a non-endogenous functional Druantia Type II defense system is selected from the group consisting of the species listed in Table 10.

According to some embodiments, the Druantia systems' (Type III) components are located in a gene cluster (a cassette of genes) in a microbial cell genome. According to some embodiments, the Druantia system components are located in close proximity (e.g. positioned within 20 genes, 10 genes, 5 genes or less one from the other) in a genome of a prokaryotic cell. According to some embodiments the prokaryotic cell expresses an endogenous Druantia Type IIII defense system. According to some embodiments, the prokaryotic cell expresses an endogenous functional Druantia Type IIII defense system. According to some embodiments, the species of prokaryotic cell is selected from the group consisting of the species listed in Table 10.

According to some embodiments a prokaryotic cell expresses a non-endogenous Druantia Type III defense system. According to some embodiments, a prokaryotic cell expresses a non-endogenous functional Druantia Type III defense system. According to some embodiments, the species of prokaryotic cell expressing a non-endogenous functional Druantia Type III defense system is selected from the group consisting of the species listed in Table 10.

In some embodiments, Druantia Type I defense system components comprise DruA, DruB, DruC, DruD, and DruE polypeptides. In some embodiments, a Druantia Type I defense system's components comprise functional portions of DruA, DruB, DruC, DruD, and DruE polypeptides.

In some embodiments, Druantia Type II defense system's components comprise DruM, DruF, DruG, and DruE polypeptides. In some embodiments, a Druantia Type II defense system's components comprise functional portions of DruM, DruF, DruG, and DruE polypeptides.

In some embodiments, Druantia Type III defense system components comprise DruH and DruE polypeptides. In some embodiments, a Druantia Type III defense system components comprise functional portions of DruH and DruE polypeptides.

In some embodiments, the Druantia Type I defense system components are encoded by druA, druB, druC, druD, and druE genes. In some embodiments, the Druantia Type I defense system components are encoded by a portion of druA, druB, druC, druD, and druE genes, where the portions of the gene encode functional portions of the respective polypeptides.

In some embodiments, the Druantia Type II defense system components are encoded by druM, druF, druG, and druE genes. In some embodiments, the Druantia Type II defense system components are encoded by a portion of druM, druF, druG, and druE genes, where the portions of the gene encode functional portions of the respective polypeptides.

In some embodiments, the Druantia Type III defense system components are encoded by druH and druE genes. In some embodiments, the Druantia Type III defense system components are encoded by a portion of druH and druE genes, where the portions of the gene encode functional portions of the respective polypeptides.

Non-limiting embodiments of endogenous Druantia systems (Defense Systems IIIa, IIIb, and IIIc) and the respective location of their components are provided in Table 10 herein.

In some embodiments, the components of a Druantia system may be identified by the presence of similar domains present within each component. In some embodiments, domains comprise pfam domains and or clusters of orthologous groups (COG) domains.

In some embodiments, the term “DruA” refers to the polynucleotide or expression product e.g., the polypeptide encoded by the druA gene. In some embodiments, the term “DruA” refers to a DruA polypeptide. In some embodiments, the druA gene encodes a polypeptide comprising a pfam14236 domain. In some embodiments, the druA gene encodes a polypeptide comprising a DUF4338 domain. In some embodiments, the druA gene encodes a polypeptide comprising a pfam14236 domain and a DUF4338 domain. In some embodiments, the DruA polypeptide comprises a pfam14236 domain. In some embodiments, the DruA polypeptide comprises a DUF4338 domain. In some embodiments, the DruA polypeptide comprises a pfam14236 domain and a DUF4338 domain.

In some embodiments, DruA polypeptide is encoded by a gene positioned within 5-60 genes of a gene encoding a DruB, DruC, DruD, or DruE polypeptide, or any combination thereof, in a genome of a prokaryotic cell. In some embodiments, DruA polypeptide is encoded by a gene positioned within 5 genes upstream (5′) or downstream (3′) to a gene encoding a DruB, DruC, DruD, or DruE polypeptide, or any combination thereof, in a genome of a prokaryotic cell. In some embodiments, DruA polypeptide is encoded by a gene positioned within 5 genes upstream (5′) and downstream (3′) to a gene encoding a DruB, DruC, DruD, or DruE, or any combination thereof, polypeptide in a genome of a prokaryotic cell.

In some embodiments, DruA and DruB are encoded by genes positioned sequentially 5′ to 3′ in a genome of a prokaryotic cell. In some embodiments, DruA and DruB are encoded by genes positioned contiguously 5′ to 3′ in a genome of a prokaryotic cell. In some embodiments, DruA, DruB, and DruC are encoded by genes positioned sequentially 5′ to 3′ in a genome of a prokaryotic cell. In some embodiments, DruA, DruB, and DruC are encoded by genes positioned contiguously 5′ to 3′ in a genome of a prokaryotic cell. In some embodiments, DruA, DruB, DruC, and DruD are encoded by genes positioned sequentially 5′ to 3′ in a genome of a prokaryotic cell. In some embodiments, DruA, DruB, DruC, and DruD are encoded by genes positioned contiguously 5′ to 3′ in a genome of a prokaryotic cell. In some embodiments, DruA, DruB, DruC, DruD, and DruE are encoded by genes positioned sequentially 5′ to 3′ in a genome of a prokaryotic cell. In some embodiments, DruA, DruB, DruC, DruD, and DruE are encoded by genes positioned contiguously 5′ to 3′ in a genome of a prokaryotic cell.

In some embodiments, druA and druB genes are positioned sequentially 5′ to 3′ in a genome of a prokaryotic cell. In some embodiments, druA and druB genes are positioned contiguously 5′ to 3′ in a genome of a prokaryotic cell. In some embodiments, druA, druB, and druC genes are positioned sequentially 5′ to 3′ in a genome of a prokaryotic cell. In some embodiments, druA, druB, and druC genes are positioned contiguously 5′ to 3′ in a genome of a prokaryotic cell. In some embodiments, druA, druB, druC, and druD genes are positioned sequentially 5′ to 3′ in a genome of a prokaryotic cell. In some embodiments, druA, druB, druC, and druD genes are positioned contiguously 5′ to 3′ in a genome of a prokaryotic cell. In some embodiments, druA, druB, druC, druD, and druE genes are positioned sequentially 5′ to 3′ in a genome of a prokaryotic cell. In some embodiments, druA, druB, druC, druD, and druE genes are positioned contiguously 5′ to 3′ in a genome of a prokaryotic cell.

In some embodiments, a DruA polypeptide is about 404 amino acids long (median gene size).

In some embodiments, the DruA polypeptide comprises the amino acid sequence having at least 80% homology to a sequence selected from the group consisting of the polypeptides referenced in Table 10, rows 2-123, columns J and K. In some embodiments, the DruA polypeptide comprises the amino acid sequence having at least 80% homology within similar domain regions within a sequence selected from the group consisting of the polypeptides referenced in Table 10, rows 2-123, columns J and K. In some embodiments, the DruA polypeptide comprises a homolog of a polypeptide having the amino acid sequences set forth in the polypeptides referenced in Table 10, rows 2-123, columns J and K.

In some embodiments, the DruA polypeptide comprises the amino acid sequence having at least 80% identity to a sequence selected from the group consisting of the polypeptides referenced in Table 10, rows 2-123, columns J and K. In some embodiments, the DruA polypeptide comprises the amino acid sequence having at least 80% identity within similar domain regions within a sequence selected from the group consisting of the polypeptides referenced in Table 10, rows 2-123, columns J and K.

In some embodiments, the DruA polypeptide comprises an amino acid sequence having at least 80%, at least 85%, at least 90%, or at least 95% homology to a sequence selected from the group consisting of the polypeptides referenced in Table 10, rows 2-123, columns J and K. In some embodiments, the DruA polypeptide comprises the amino acid sequence having at least 80%, 85%, 90%, 95% homology within similar domain regions within a sequence selected from the group consisting of the polypeptides referenced in Table 10, rows 2-123, columns J and K. In some embodiments, the DruA polypeptide comprises an amino acid sequence having at least 80%, at least 85%, at least 90%, or at least 95% identity to a sequence selected from the group consisting of the polypeptides referenced in Table 10, rows 2-123, columns J and K. In some embodiments, the DruA polypeptide comprises the amino acid sequence having at least 80%, 85%, 90%, 95% identity within similar domain regions within a sequence selected from the group consisting of the polypeptides referenced in Table 10, rows 2-123, columns J and K. In some embodiments, the DruA polypeptide comprises an amino acid sequence selected from the group consisting of the polypeptides referenced in Table 10, rows 2-123, columns J and K.

In some embodiments, the DruA polypeptide comprises an amino acid sequence encoded by a nucleic acid sequence having at least 80%, at least 85%, at least 90%, or at least 95% homology to a sequence selected from the group consisting of the polynucleotides referenced in Table 10, rows 2-123, columns J and L. In some embodiments, the DruA polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 2-123, columns J and L. In some embodiments, the DruA polypeptide comprises an amino acid sequence encoded by a nucleic acid sequence having at least 80%, at least 85%, at least 90%, or at least 95% identity to a sequence selected from the group consisting of the polynucleotides referenced in Table 10, rows 2-123, columns J and L. In some embodiments, the DruA polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 2-123, columns J and L. In some embodiments, the DruA polypeptide comprises an amino acid sequence encoded from a nucleic acid sequence selected from the group consisting of the polynucleotides referenced in Table 10, rows 2-123, columns J and L.

In some embodiments, the nucleic acid sequence of a druA gene comprises a nucleic acid sequence having at least 80%, at least 85%, at least 90%, or at least 95% homology to a sequence selected from the group consisting of the polynucleotides referenced in Table 10, rows 2-123, columns J and L. In some embodiments, the druA gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 10, rows 2-123, columns J and L. In some embodiments, the nucleic acid sequence of a druA gene comprises a nucleic acid sequence having at least 80%, at least 85%, at least 90%, or at least 95% identity to a sequence selected from the group consisting of the polynucleotides referenced in Table 10, rows 2-123, columns J and L. In some embodiments, the druA gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 10, rows 2-123, columns J and L. In some embodiments, the nucleic acid sequence of a druA gene comprises a nucleic acid sequence selected from the group consisting of the polynucleotides referenced in Table 10, rows 2-123, columns J and L.

As used herein, the term “DruB” refers to the polynucleotide or expression product e.g., polypeptide encoded by the druB gene. In some embodiments, the term “DruB” refers to a DruB polypeptide.

In some embodiments, DruB polypeptide is encoded by a gene positioned within 5-60 genes of a gene encoding a DruC, a DruD, or a DruE polypeptide, or any combination thereof in a genome of a prokaryotic cell. In some embodiments, a DruB-DruC fusion polypeptide is encoded by a gene positioned within 5-60 genes of a gene encoding a DruD or a DruE polypeptide or a combination thereof. In some embodiments, DruB polypeptide is encoded by a gene positioned within 5 genes upstream (5′) or downstream (3′) to a gene encoding a DruC, a DruD, or a DruE polypeptide or any combination thereof in a genome of a prokaryotic cell. In some embodiments, DruB-DruC fusion polypeptide is encoded by a gene positioned within 5 genes upstream (5′) or downstream (3′) to a gene encoding a DruD or a DruE polypeptide or a combination thereof in a genome of a prokaryotic cell. In some embodiments, DruB polypeptide is encoded by a gene positioned within 5 genes upstream (5′) and downstream (3′) to a gene encoding a DruC, a DruD, or a DruE polypeptide or a combination thereof in a genome of a prokaryotic cell.

In some embodiments, DruB and DruC are encoded by genes positioned sequentially 5′ to 3′ in a genome of a prokaryotic cell. In some embodiments DruB and DruC are encoded by genes positioned contiguously 5′ to 3′ in a genome of a prokaryotic cell. In some embodiments, DruB, DruC, and DruD are encoded by genes positioned sequentially 5′ to 3′ in a genome of a prokaryotic cell. In some embodiments DruB, DruC, and DruD are encoded by genes positioned contiguously 5′ to 3′ in a genome of a prokaryotic cell. In some embodiments, DruB, DruC, DruD, and DruE are encoded by genes positioned sequentially 5′ to 3′ in a genome of a prokaryotic cell. In some embodiments DruB, DruC, DruD, and DruE are encoded by genes positioned contiguously 5′ to 3′ in a genome of a prokaryotic cell. In some embodiments, DruB, DruC, and DruD are encoded by genes positioned sequentially 5′ to 3′ in a genome of a prokaryotic cell. In some embodiments DruB, DruC, and DruD are encoded by genes positioned contiguously 5′ to 3′ in a genome of a prokaryotic cell. In some embodiments, DruB, DruC, DruD, DruE, and DruA are encoded by genes positioned sequentially 5′ to 3′ in a genome of a prokaryotic cell. In some embodiments DruB, DruC, DruD, DruE, and DruA are encoded by genes positioned contiguously 5′ to 3′ in a genome of a prokaryotic cell.

In some embodiments, druB and druC genes are positioned sequentially 5′ to 3′ in a genome of a prokaryotic cell. In some embodiments, druB and druC genes are positioned contiguously 5′ to 3′ in a genome of a prokaryotic cell. In some embodiments, druB, druC, and druD genes are positioned sequentially 5′ to 3′ in a genome of a prokaryotic cell. In some embodiments, druB, druC, and druD genes are positioned contiguously 5′ to 3′ in a genome of a prokaryotic cell. In some embodiments, druB, druC, druD, and druE genes are positioned sequentially 5′ to 3′ in a genome of a prokaryotic cell. In some embodiments, druB, druC, druD, and druE genes are positioned contiguously 5′ to 3′ in a genome of a prokaryotic cell. In some embodiments, druB, druC, druD, druE, and druA genes are positioned sequentially 5′ to 3′ in a genome of a prokaryotic cell. In some embodiments, druB, druC, druD, druE, and druA genes are positioned contiguously 5′ to 3′ in a genome of a prokaryotic cell.

In some embodiments, a DruB polypeptide is about 531 amino acids long (median gene size).

In some embodiments, the DruB polypeptide comprises the amino acid sequence having at least 80% homology to a sequence selected from the group consisting of the polypeptides referenced in Table 10, rows 2-123, columns N and O. In some embodiments, the DruB polypeptide comprises the amino acid sequence having at least 80% homology within similar domain regions within a sequence selected from the group consisting of the polypeptides referenced in Table 10, rows 2-123, columns N and O. In some embodiments, the DruB polypeptide comprises a homolog of a polypeptide having the amino acid sequences set forth in the polypeptides referenced in Table 10, rows 2-123, columns N and O.

In some embodiments, the DruB polypeptide comprises the amino acid sequence having at least 80% identity to a sequence selected from the group consisting of the polypeptides referenced in Table 10, rows 2-123, columns N and O. In some embodiments, the DruB polypeptide comprises the amino acid sequence having at least 80% identity within similar domain regions within a sequence selected from the group consisting of the polypeptides referenced in Table 10, rows 2-123, columns N and O.

In some embodiments, the DruB polypeptide comprises an amino acid sequence having at least 80%, at least 85%, at least 90%, or at least 95% homology to a sequence selected from the group consisting of the polypeptides referenced in Table 10, rows 2-123, columns N and O. In some embodiments, the DruB polypeptide comprises the amino acid sequence having at least 80%, 85%, 90%, 95% homology within similar domain regions within a sequence selected from the group consisting of the polypeptides referenced in Table 10, rows 2-123, columns N and O. In some embodiments, the DruB polypeptide comprises an amino acid sequence having at least 80%, at least 85%, at least 90%, or at least 95% identity to a sequence selected from the group consisting of the polypeptides referenced in Table 10, rows 2-123, columns N and O. In some embodiments, the DruB polypeptide comprises the amino acid sequence having at least 80%, 85%, 90%, 95% identity within similar domain regions within a sequence selected from the group consisting of the polypeptides referenced in Table 10, rows 2-123, columns N and O. In some embodiments, the DruB polypeptide comprises an amino acid sequence selected from the group consisting of the polypeptides referenced in Table 10, rows 2-123, columns N and O.

In some embodiments, the DruB polypeptide comprises an amino acid sequence encoded by a nucleic acid sequence having at least 80%, at least 85%, at least 90%, or at least 95% homology to a sequence selected from the group consisting of the polynucleotides referenced in Table 10, rows 2-123, columns N and P. In some embodiments, the DruB polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 2-123, columns N and P. In some embodiments, the DruB polypeptide comprises an amino acid sequence encoded by a nucleic acid sequence having at least 80%, at least 85%, at least 90%, or at least 95% identity to a sequence selected from the group consisting of the polynucleotides referenced in Table 10, rows 2-123, columns N and P. In some embodiments, the DruB polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 2-123, columns N and P. In some embodiments, the DruB polypeptide comprises an amino acid sequence encoded from a nucleic acid sequence selected from the group consisting of the polynucleotides referenced in Table 10, rows 2-123, columns N and P.

In some embodiments, the nucleic acid sequence of a druB gene comprises a nucleic acid sequence having at least 80%, at least 85%, at least 90%, or at least 95% homology to a sequence selected from the group consisting of the polynucleotides referenced in Table 10, rows 2-123, columns N and P. In some embodiments, the druB gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 10, rows 2-123, columns N and P. In some embodiments, the nucleic acid sequence of a druB gene comprises a nucleic acid sequence having at least 80%, at least 85%, at least 90%, or at least 95% identity to a sequence selected from the group consisting of the polynucleotides referenced in Table 10, rows 2-123, columns N and P. In some embodiments, the druB gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 10, rows 2-123, column L. In some embodiments, the nucleic acid sequence of a druB gene comprises a nucleic acid sequence selected from the group consisting of the polynucleotides referenced in Table 10, rows 2-123, columns N and P.

In some embodiments, the term “DruC” refers to the polynucleotide or expression product e.g., the polypeptide encoded by the druC gene. In some embodiments, the term “DruC” refers to a DruC polypeptide.

In some embodiments, DruC polypeptide is encoded by a gene positioned within 5-60 genes of a gene encoding a DruD or DruE polypeptide, or any combination thereof, in a genome of a prokaryotic cell. In some embodiments, DruC polypeptide is encoded by a gene positioned within 5 genes upstream (5′) or downstream (3′) to a gene encoding a DruD or DruE polypeptide, or any combination thereof, in a genome of a prokaryotic cell. In some embodiments, a DruC polypeptide is encoded by a gene positioned within 5 genes upstream (5′) and downstream (3′) to a gene encoding a DruD or DruE polypeptide in a genome of a prokaryotic cell.

In some embodiments, DruC and DruD are encoded by genes positioned sequentially 5′ to 3′ in a genome of a prokaryotic cell. In some embodiments, DruC and DruD are encoded by genes positioned contiguously 5′ to 3′ in a genome of a prokaryotic cell. In some embodiments, DruC, DruD, and DruE are encoded by genes positioned sequentially 5′ to 3′ in a genome of a prokaryotic cell. In some embodiments, DruC, DruD, and DruE are encoded by genes positioned contiguously 5′ to 3′ in a genome of a prokaryotic cell. In some embodiments, DruC, DruD, DruE, and DruA are encoded by genes positioned sequentially 5′ to 3′ in a genome of a prokaryotic cell. In some embodiments, DruC, DruD, DruE, and DruA are encoded by genes positioned contiguously 5′ to 3′ in a genome of a prokaryotic cell.

In some embodiments, DruC and DruE polypeptides are encoded by genes positioned sequentially 5′ to 3′ in a genome of a prokaryotic cell. In some embodiments, DruC and DruE polypeptides are encoded by genes positioned contiguously 5′ to 3′ in a genome of a prokaryotic cell.

In some embodiments, druC and druD genes are positioned sequentially 5′ to 3′ in a genome of a prokaryotic cell. In some embodiments, druC and druD genes are positioned contiguously 5′ to 3′ in a genome of a prokaryotic cell. In some embodiments, druC, druD, and druE genes are positioned sequentially 5′ to 3′ in a genome of a prokaryotic cell. In some embodiments, druC, druD, and druE genes are positioned contiguously 5′ to 3′ in a genome of a prokaryotic cell. In some embodiments, druC, druD, druE, and druA genes are positioned sequentially 5′ to 3′ in a genome of a prokaryotic cell. In some embodiments, druC, druD, druE, and druA genes are positioned contiguously 5′ to 3′ in a genome of a prokaryotic cell.

In some embodiments, druC and druE genes are positioned sequentially 5′ to 3′ in a genome of a prokaryotic cell. In some embodiments, druC and druE genes are positioned contiguously 5′ to 3′ in a genome of a prokaryotic cell. In some embodiments, druC, druE, and druA genes are positioned sequentially 5′ to 3′ in a genome of a prokaryotic cell. In some embodiments, druC, druE, and druA genes are positioned contiguously 5′ to 3′ in a genome of a prokaryotic cell. In some embodiments, druC and druA genes are positioned sequentially 5′ to 3′ in a genome of a prokaryotic cell. In some embodiments, druC and druA genes are positioned contiguously 5′ to 3′ in a genome of a prokaryotic cell.

In some embodiments, a DruC polypeptide is about 627 amino acids long (median gene size).

In some embodiments, the DruC polypeptide comprises the amino acid sequence having at least 80% homology to a sequence selected from the group consisting of the polypeptides referenced in Table 10, rows 2-123, columns R and S. In some embodiments, the DruC polypeptide comprises the amino acid sequence having at least 80% homology within similar domain regions within a sequence selected from the group consisting of the polypeptides referenced in Table 10, rows 2-123, columns R and S. In some embodiments, the DruC polypeptide comprises a homolog of a polypeptide having the amino acid sequences set forth in the polypeptides referenced in Table 10, rows 2-123, columns R and S.

In some embodiments, the DruC polypeptide comprises the amino acid sequence having at least 80% identity to a sequence selected from the group consisting of the polypeptides referenced in Table 10, rows 2-123, columns R and S. In some embodiments, the DruC polypeptide comprises the amino acid sequence having at least 80% identity within similar domain regions within a sequence selected from the group consisting of the polypeptides referenced in Table 10, rows 2-123, columns R and S.

In some embodiments, the DruC polypeptide comprises an amino acid sequence having at least 80%, at least 85%, at least 90%, or at least 95% homology to a sequence selected from the group consisting of the polypeptides referenced in Table 10, rows 2-123, columns R and S. In some embodiments, the DruC polypeptide comprises the amino acid sequence having at least 80%, 85%, 90%, 95% homology within similar domain regions within a sequence selected from the group consisting of the polypeptides referenced in Table 10, rows 2-123, columns R and S. In some embodiments, the DruC polypeptide comprises an amino acid sequence having at least 80%, at least 85%, at least 90%, or at least 95% identity to a sequence selected from the group consisting of the polypeptides referenced in Table 10, rows 2-123, columns R and S. In some embodiments, the DruC polypeptide comprises the amino acid sequence having at least 80%, 85%, 90%, 95% identity within similar domain regions within a sequence selected from the group consisting of the polypeptides referenced in Table 10, rows 2-123, columns R and S. In some embodiments, the DruC polypeptide comprises an amino acid sequence selected from the group consisting of the polypeptides referenced in Table 10, rows 2-123, columns R and S.

In some embodiments, the DruC polypeptide comprises an amino acid sequence encoded by a nucleic acid sequence having at least 80%, at least 85%, at least 90%, or at least 95% homology to a sequence selected from the group consisting of the polynucleotides referenced in Table 10, rows 2-123, columns R and T. In some embodiments, the DruC polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 2-123, columns R and T. In some embodiments, the DruC polypeptide comprises an amino acid sequence encoded by a nucleic acid sequence having at least 80%, at least 85%, at least 90%, or at least 95% identity to a sequence selected from the group consisting of the polynucleotides referenced in Table 10, rows 2-123, columns R and T. In some embodiments, the DruC polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 2-123, columns R and T. In some embodiments, the DruC polypeptide comprises an amino acid sequence encoded from a nucleic acid sequence selected from the group consisting of the polynucleotides referenced in Table 10, rows 2-123, columns R and T.

In some embodiments, the nucleic acid sequence of a druC gene comprises a nucleic acid sequence having at least 80%, at least 85%, at least 90%, or at least 95% homology to a sequence selected from the group consisting of the polynucleotides referenced in Table 10, rows 2-123, columns R and T. In some embodiments, the druC gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 10, rows 2-123, columns R and T. In some embodiments, the nucleic acid sequence of a druC gene comprises a nucleic acid sequence having at least 80%, at least 85%, at least 90%, or at least 95% identity to a sequence selected from the group consisting of the polynucleotides referenced in Table 10, rows 2-123, columns R and T. In some embodiments, the druC gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 10, rows 2-123, columns R and T. In some embodiments, the nucleic acid sequence of a druC gene comprises a nucleic acid sequence selected from the group consisting of the polynucleotides referenced in Table 10, rows 2-123, columns R and T.

In some embodiments, the term “DruD” refers to the polynucleotide or expression product e.g., the polypeptide encoded by the druD gene. In some embodiments, the term “DruD” refers to a DruD polypeptide.

In some embodiments, DruD polypeptide is encoded by a gene positioned within 5-60 genes of a gene encoding a DruE or DruA polypeptide, or any combination thereof, in a genome of a prokaryotic cell. In some embodiments, DruD polypeptide is encoded by a gene positioned within 5 genes upstream (5′) or downstream (3′) to a gene encoding a DruE or DruA polypeptide, or any combination thereof, in a genome of a prokaryotic cell.

In some embodiments, DruD and DruE are encoded by genes positioned sequentially 5′ to 3′ in a genome of a prokaryotic cell. In some embodiments, DruD and DruE are encoded by genes positioned contiguously 5′ to 3′ in a genome of a prokaryotic cell. In some embodiments, DruD, DruE, and DruA are encoded by genes positioned sequentially 5′ to 3′ in a genome of a prokaryotic cell. In some embodiments, DruD, DruE, and DruA are encoded by genes positioned contiguously 5′ to 3′ in a genome of a prokaryotic cell.

In some embodiments, druD and druE genes are positioned sequentially 5′ to 3′ in a genome of a prokaryotic cell. In some embodiments, druD and druE genes are positioned contiguously 5′ to 3′ in a genome of a prokaryotic cell. In some embodiments, druD, druE, and druA genes are positioned sequentially 5′ to 3′ in a genome of a prokaryotic cell. In some embodiments, druD, druE, and druA genes are positioned contiguously 5′ to 3′ in a genome of a prokaryotic cell.

In some embodiments, druD and druA genes are positioned sequentially 5′ to 3′ in a genome of a prokaryotic cell. In some embodiments, druD and druA genes are positioned contiguously 5′ to 3′ in a genome of a prokaryotic cell.

In some embodiments, a DruD polypeptide is about 347 amino acids long (median gene size).

In some embodiments, the DruD polypeptide comprises the amino acid sequence having at least 80% homology to a sequence selected from the group consisting of the polypeptides referenced in Table 10, rows 2-123, columns V and W. In some embodiments, the DruD polypeptide comprises the amino acid sequence having at least 80% homology within similar domain regions within a sequence selected from the group consisting of the polypeptides referenced in Table 10, rows 2-123, columns V and W. In some embodiments, the DruD polypeptide comprises a homolog of a polypeptide having the amino acid sequences set forth in the polypeptides referenced in Table 10, rows 2-123, columns V and W.

In some embodiments, the DruD polypeptide comprises the amino acid sequence having at least 80% identity to a sequence selected from the group consisting of the polypeptides referenced in Table 10, rows 2-123, columns V and W. In some embodiments, the DruD polypeptide comprises the amino acid sequence having at least 80% identity within similar domain regions within a sequence selected from the group consisting of the polypeptides referenced in Table 10, rows 2-123, columns V and W.

In some embodiments, the DruD polypeptide comprises an amino acid sequence having at least 80%, at least 85%, at least 90%, or at least 95% homology to a sequence selected from the group consisting of the polypeptides referenced in Table 10, rows 2-123, columns V and W. In some embodiments, the DruD polypeptide comprises the amino acid sequence having at least 80%, 85%, 90%, 95% homology within similar domain regions within a sequence selected from the group consisting of the polypeptides referenced in Table 10, rows 2-123, columns V and W. In some embodiments, the DruD polypeptide comprises an amino acid sequence having at least 80%, at least 85%, at least 90%, or at least 95% identity to a sequence selected from the group consisting of the polypeptides referenced in Table 10, rows 2-123, columns V and W. In some embodiments, the DruD polypeptide comprises the amino acid sequence having at least 80%, 85%, 90%, 95% identity within similar domain regions within a sequence selected from the group consisting of the polypeptides referenced in Table 10, rows 2-123, columns V and W. In some embodiments, the DruD polypeptide comprises an amino acid sequence selected from the group consisting of the polypeptides referenced in Table 10, rows 2-123, columns V and W.

In some embodiments, the DruD polypeptide comprises an amino acid sequence encoded by a nucleic acid sequence having at least 80%, at least 85%, at least 90%, or at least 95% homology to a sequence selected from the group consisting of the polynucleotides referenced in Table 10, rows 2-123, columns V and X. In some embodiments, the DruD polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 2-123, columns V and X. In some embodiments, the DruD polypeptide comprises an amino acid sequence encoded by a nucleic acid sequence having at least 80%, at least 85%, at least 90%, or at least 95% identity to a sequence selected from the group consisting of the polynucleotides referenced in Table 10, rows 2-123, columns V and X. In some embodiments, the DruD polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 2-123, columns V and X. In some embodiments, the DruD polypeptide comprises an amino acid sequence encoded from a nucleic acid sequence selected from the group consisting of the polynucleotides referenced in Table 10, rows 2-123, columns V and X.

In some embodiments, the nucleic acid sequence of a druD gene comprises a nucleic acid sequence having at least 80%, at least 85%, at least 90%, or at least 95% homology to a sequence selected from the group consisting of the polynucleotides referenced in Table 10, rows 2-123, column P. In some embodiments, the druD gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 10, rows 2-123, columns V and X. In some embodiments, the nucleic acid sequence of a druD gene comprises a nucleic acid sequence having at least 80%, at least 85%, at least 90%, or at least 95% identity to a sequence selected from the group consisting of the polynucleotides referenced in Table 10, rows 2-123, columns V and X. In some embodiments, the druD gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 10, rows 2-123, columns V and X. In some embodiments, the nucleic acid sequence of a druD gene comprises a nucleic acid sequence selected from the group consisting of the polynucleotides referenced in Table 10, rows 2-123, columns V and X.

In some embodiments, the term “DruE” refers to the polynucleotide or expression product e.g., the polypeptide encoded by the druE gene. In some embodiments, the term “DruE” refers to a DruE polypeptide. In some embodiments, the druE gene encodes a polypeptide comprising a pfam00270 domain. In some embodiments, the druE gene encodes a polypeptide comprising a pfam00271 domain. In some embodiments, the druE gene encodes a polypeptide comprising a pfam09369 domain. In some embodiments, the druE gene encodes a polypeptide comprising a DUF1998 domain. In some embodiments, the druE gene encodes a polypeptide comprising a pfam00270 domain, a pfam00271 domain, a pfam09369 domain, or a DUF4338 domain, or any combination thereof. In some embodiments, the druE gene encodes a polypeptide comprising a pfam00270 domain, a pfam00271 domain, a pfam09369 domain, and a DUF4338 domain. In some embodiments, a druE gene encodes a polypeptide comprising a helicase activity. In some embodiments, a druE gene encodes a polypeptide comprising a DEAx box helicase activity.

In some embodiments, the DruE polypeptide comprises a pfam00270 domain. In some embodiments, the DruE polypeptide comprises a pfam00271 domain. In some embodiments, the DruE polypeptide comprises a pfam09369 domain. In some embodiments, the DruE polypeptide comprises a DUF1998 domain. In some embodiments, the DruE polypeptide comprises a pfam00270 domain, a pfam00271 domain, a pfam09369 domain, or a DUF4338 domain, or any combination thereof. In some embodiments, the DruE polypeptide comprises a pfam00270 domain, a pfam00271 domain, a pfam09369 domain, and a DUF4338 domain. In some embodiments, a DruE polypeptide comprises a helicase activity. In some embodiments, a DruE polypeptide comprises a DEAx box helicase activity.

In some embodiments, DruE polypeptide is encoded by a gene positioned within 5-60 genes of a gene encoding a DruA polypeptide in a genome of a prokaryotic cell. In some embodiments, DruE polypeptide is encoded by a gene positioned within 5 genes upstream (5′) or downstream (3′) to a gene encoding a DruA polypeptide in a genome of a prokaryotic cell. In some embodiments, DruE polypeptide is encoded by a gene positioned within 5 genes upstream (5′) and downstream (3′) to a gene encoding a DruA polypeptide in a genome of a prokaryotic cell.

In some embodiments, DruE and DruA are encoded by genes positioned sequentially 5′ to 3′ in a genome of a prokaryotic cell. In some embodiments, DruE and DruA are encoded by genes positioned contiguously 5′ to 3′ in a genome of a prokaryotic cell.

In some embodiments, druE and druA genes are positioned sequentially 5′ to 3′ in a genome of a prokaryotic cell. In some embodiments, druE and druA genes are positioned contiguously 5′ to 3′ in a genome of a prokaryotic cell.

In some embodiments, a DruE polypeptide is about 2104 amino acids long (median gene size).

In some embodiments, the DruE polypeptide comprises the amino acid sequence having at least 80% homology to a sequence selected from the group consisting of the polypeptides referenced in Table 10, rows 2-1343, columns Z and AA. In some embodiments, the DruE polypeptide comprises the amino acid sequence having at least 80% homology within similar domain regions within a sequence selected from the group consisting of the polypeptides referenced in Table 10, rows 2-1343, columns Z and AA. In some embodiments, the DruE polypeptide comprises a homolog of a polypeptide having the amino acid sequences set forth in the polypeptides referenced in Table 10, rows 2-1343, columns Z and AA.

In some embodiments, a DruE polypeptide homolog comprises a helicase domain. In some embodiments, a DruE polypeptide comprises a helicase activity.

In some embodiments, the DruE polypeptide comprises the amino acid sequence having at least 80% identity to a sequence selected from the group consisting of the polypeptides referenced in Table 10, rows 2-1343, columns Z and AA. In some embodiments, the DruE polypeptide comprises the amino acid sequence having at least 80% identity within similar domain regions within a sequence selected from the group consisting of the polypeptides referenced in Table 10, rows 2-1343, columns Z and AA.

In some embodiments, the DruE polypeptide comprises an amino acid sequence having at least 80%, at least 85%, at least 90%, or at least 95% homology to a sequence selected from the group consisting of the polypeptides referenced in Table 10, rows 2-1343, columns Z and AA. In some embodiments, the DruE polypeptide comprises the amino acid sequence having at least 80%, 85%, 90%, 95% homology within similar domain regions within a sequence selected from the group consisting of the polypeptides referenced in Table 10, rows 2-1343, columns Z and AA. In some embodiments, the DruE polypeptide comprises an amino acid sequence having at least 80%, at least 85%, at least 90%, or at least 95% identity to a sequence selected from the group consisting of the polypeptides referenced in Table 10, rows 2-1343, columns Z and AA. In some embodiments, the DruE polypeptide comprises the amino acid sequence having at least 80%, 85%, 90%, 95% identity within similar domain regions within a sequence selected from the group consisting of the polypeptides referenced in Table 10, rows 2-1343, columns Z and AA. In some embodiments, the DruE polypeptide comprises an amino acid sequence selected from the group consisting of the polypeptides referenced in Table 10, rows 2-1343, columns Z and AA.

In some embodiments, the DruE polypeptide comprises an amino acid sequence encoded by a nucleic acid sequence having at least 80%, at least 85%, at least 90%, or at least 95% homology to a sequence selected from the group consisting of the polynucleotides referenced in Table 10, rows 2-1343, columns Z and AB. In some embodiments, the DruE polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 2-1343, columns Z and AB. In some embodiments, the DruE polypeptide comprises an amino acid sequence encoded by a nucleic acid sequence having at least 80%, at least 85%, at least 90%, or at least 95% identity to a sequence selected from the group consisting of the polynucleotides referenced in Table 10, rows 2-1343, columns Z and AB. In some embodiments, the DruE polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 2-1343, columns Z and AB. In some embodiments, the DruE polypeptide comprises an amino acid sequence encoded from a nucleic acid sequence selected from the group consisting of the polynucleotides referenced in Table 10, rows 2-1343, columns Z and AB.

In some embodiments, the nucleic acid sequence of a druE gene comprises a nucleic acid sequence having at least 80%, at least 85%, at least 90%, or at least 95% homology to a sequence selected from the group consisting of the polynucleotides referenced in Table 10, rows 2-1343, columns Z and AB. In some embodiments, the druE gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 10, rows 2-1343, columns Z and AB. In some embodiments, the nucleic acid sequence of a druE gene comprises a nucleic acid sequence having at least 80%, at least 85%, at least 90%, or at least 95% identity to a sequence selected from the group consisting of the polynucleotides referenced in Table 10, rows 2-1343, columns Z and AB. In some embodiments, the druE gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 10, rows 2-1343, columns Z and AB. In some embodiments, the nucleic acid sequence of a druE gene comprises a nucleic acid sequence selected from the group consisting of the polynucleotides referenced in Table 10, rows 2-1343, columns Z and AB.

In some embodiments, the term “DruM” refers to the polynucleotide or expression product e.g., the polypeptide encoded by the druM gene. In some embodiments, the term “DruM” refers to a DruM polypeptide. In some embodiments, the druM gene encodes a polypeptide.

In some embodiments, DruM polypeptide is encoded by a gene positioned within 5-60 genes of a gene encoding a DruF polypeptide, a DruG polypeptide, and/or a DruE polypeptide in a genome of a prokaryotic cell. In some embodiments, DruM polypeptide is encoded by a gene positioned within 5 genes upstream (5′) or downstream (3′) to a gene encoding a DruF polypeptide, a DruG polypeptide, and/or a DruE polypeptide in a genome of a prokaryotic cell. In some embodiments, DruM polypeptide is encoded by a gene positioned within 5 genes upstream (5′) and downstream (3′) to a gene encoding a DruF polypeptide, a DruG polypeptide, and/or a DruE polypeptide in a genome of a prokaryotic cell.

In some embodiments, DruM. DruF, DruG, and DruE are encoded by genes positioned sequentially 5′ to 3′ in a genome of a prokaryotic cell. In some embodiments, DruM, DruF, DruG, and DruE are encoded by genes positioned contiguously 5′ to 3′ in a genome of a prokaryotic cell.

In some embodiments, druM, druF, druG, and druE genes are positioned sequentially 5′ to 3′ in a genome of a prokaryotic cell. In some embodiments, druM, druF, druG, and druE genes are positioned contiguously 5′ to 3′ in a genome of a prokaryotic cell.

In some embodiments, a DruM polypeptide is about 386 amino acids long (median gene size).

In some embodiments, the DruM polypeptide comprises the amino acid sequence having at least 80% homology to a sequence selected from the group consisting of the polypeptides referenced in Table 10, rows 124-295, columns AD and AE. In some embodiments, the DruM polypeptide comprises the amino acid sequence having at least 80% homology within similar domain regions within a sequence selected from the group consisting of the polypeptides referenced in Table 10, rows 124-295, columns AD and AE. In some embodiments, the DruM polypeptide comprises a homolog of a polypeptide having the amino acid sequences set forth in the polypeptides referenced in Table 10, rows 124-295, columns AD and AE.

In some embodiments, a DruM polypeptide homolog comprises a cytosine methylase activity. In some embodiments, a DruM polypeptide homolog comprises a cytosine methylase.

In some embodiments, the DruM polypeptide comprises the amino acid sequence having at least 80% identity to a sequence selected from the group consisting of the polypeptides referenced in Table 10, rows 124-295, columns AD and AE. In some embodiments, the DruM polypeptide comprises the amino acid sequence having at least 80% identity within similar domain regions within a sequence selected from the group consisting of the polypeptides referenced in Table 10, rows 124-295, columns AD and AE.

In some embodiments, the DruM polypeptide comprises an amino acid sequence having at least 80%, at least 85%, at least 90%, or at least 95% homology to a sequence selected from the group consisting of the polypeptides referenced in Table 10, rows 124-295, columns AD and AE. In some embodiments, the DruM polypeptide comprises the amino acid sequence having at least 80%, 85%, 90%, 95% homology within similar domain regions within a sequence selected from the group consisting of the polypeptides referenced in Table 10, rows 124-295, columns AD and AE. In some embodiments, the DruM polypeptide comprises an amino acid sequence having at least 80%, at least 85%, at least 90%, or at least 95% identity to a sequence selected from the group consisting of the polypeptides referenced in Table 10, rows 124-295, columns AD and AE. In some embodiments, the DruM polypeptide comprises the amino acid sequence having at least 80%, 85%, 90%, 95% identity within similar domain regions within a sequence selected from the group consisting of the polypeptides referenced in Table 10, rows 124-295, columns AD and AE. In some embodiments, the DruM polypeptide comprises an amino acid sequence selected from the group consisting of the polypeptides referenced in Table 10, rows 124-295, columns AD and AE.

In some embodiments, the DruM polypeptide comprises an amino acid sequence encoded by a nucleic acid sequence having at least 80%, at least 85%, at least 90%, or at least 95% homology to a sequence selected from the group consisting of the polynucleotides referenced in Table 10, rows 124-295, columns AD and AF. In some embodiments, the DruM polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 124-295, columns AD and AF. In some embodiments, the DruM polypeptide comprises an amino acid sequence encoded by a nucleic acid sequence having at least 80%, at least 85%, at least 90%, or at least 95% identity to a sequence selected from the group consisting of the polynucleotides referenced in Table 10, rows 124-295, columns AD and AF. In some embodiments, the DruM polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 124-295, columns AD and AF. In some embodiments, the DruM polypeptide comprises an amino acid sequence encoded from a nucleic acid sequence selected from the group consisting of the polynucleotides referenced in Table 10, rows 124-295, columns AD and AF.

In some embodiments, the nucleic acid sequence of a druM gene comprises a nucleic acid sequence having at least 80%, at least 85%, at least 90%, or at least 95% homology to a sequence selected from the group consisting of the polynucleotides referenced in Table 10, rows 124-295, columns AD and AF. In some embodiments, the druM gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 10, rows 124-295, columns AD and AF. In some embodiments, a druM gene homolog encodes a cytosine methylase. In some embodiments, the nucleic acid sequence of a druM gene comprises a nucleic acid sequence having at least 80%, at least 85%, at least 90%, or at least 95% identity to a sequence selected from the group consisting of the polynucleotides referenced in Table 10, rows 124-295, columns AD and AF. In some embodiments, the druM gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 10, rows 124-295, columns AD and AF. In some embodiments, the nucleic acid sequence of a druM gene comprises a nucleic acid sequence selected from the group consisting of the polynucleotides referenced in Table 10, rows 124-295, columns AD and AF.

In some embodiments, the term “DruF” refers to the polynucleotide or expression product e.g., the polypeptide encoded by the druF gene. In some embodiments, the term “DruF” refers to a DruF polypeptide. In some embodiments, the druF gene encodes a polypeptide.

In some embodiments, DruF polypeptide is encoded by a gene positioned within 5-60 genes of a gene encoding a DruM polypeptide, a DruG polypeptide, and or a DruE polypeptide in a genome of a prokaryotic cell. In some embodiments, DruF polypeptide is encoded by a gene positioned within 5 genes upstream (5′) or downstream (3′) to a gene encoding a DruM polypeptide, a DruG polypeptide, and or a DruE polypeptide in a genome of a prokaryotic cell. In some embodiments, DruF polypeptide is encoded by a gene positioned within 5 genes upstream (5′) and downstream (3′) to a gene encoding DruM polypeptide, a DruG polypeptide, and or a DruE polypeptide in a genome of a prokaryotic cell.

In some embodiments, DruF, DruG, and DruE are encoded by genes positioned sequentially 5′ to 3′ in a genome of a prokaryotic cell. In some embodiments, DruF, DruG, and DruE are encoded by genes positioned contiguously 5′ to 3′ in a genome of a prokaryotic cell.

In some embodiments, druF, druG, and druE genes are positioned sequentially 5′ to 3′ in a genome of a prokaryotic cell. In some embodiments, druF, druG, and druE genes are positioned contiguously 5′ to 3′ in a genome of a prokaryotic cell.

In some embodiments, a DruF polypeptide is about 852 amino acids long (median gene size).

In some embodiments, the DruF polypeptide comprises the amino acid sequence having at least 80% homology to a sequence selected from the group consisting of the polypeptides referenced in Table 10, rows 124-295, columns AH and AI. In some embodiments, the DruF polypeptide comprises the amino acid sequence having at least 80% homology within similar domain regions within a sequence selected from the group consisting of the polypeptides referenced in Table 10, rows 124-295, columns AH and AI. In some embodiments, the DruF polypeptide comprises a homolog of a polypeptide having the amino acid sequences set forth in the polypeptides referenced in Table 10, rows 124-295, columns AH and AI.

In some embodiments, the DruF polypeptide comprises the amino acid sequence having at least 80% identity to a sequence selected from the group consisting of the polypeptides referenced in Table 10, rows 124-295, columns AH and AI. In some embodiments, the DruF polypeptide comprises the amino acid sequence having at least 80% identity within similar domain regions within a sequence selected from the group consisting of the polypeptides referenced in Table 10, rows 124-295, columns AH and AI.

In some embodiments, the DruF polypeptide comprises an amino acid sequence having at least 80%, at least 85%, at least 90%, or at least 95% homology to a sequence selected from the group consisting of the polypeptides referenced in Table 10, rows 124-295, columns AH and AI. In some embodiments, the DruF polypeptide comprises the amino acid sequence having at least 80%, 85%, 90%, 95% homology within similar domain regions within a sequence selected from the group consisting of the polypeptides referenced in Table 10, rows 124-295, columns AH and AI. In some embodiments, the DruF polypeptide comprises an amino acid sequence having at least 80%, at least 85%, at least 90%, or at least 95% identity to a sequence selected from the group consisting of the polypeptides referenced in Table 10, rows 124-295, columns AH and AI. In some embodiments, the DruF polypeptide comprises the amino acid sequence having at least 80%, 85%, 90%, 95% identity within similar domain regions within a sequence selected from the group consisting of the polypeptides referenced in Table 10, rows 124-295, columns AH and AI. In some embodiments, the DruF polypeptide comprises an amino acid sequence selected from the group consisting of the polypeptides referenced in Table 10, rows 124-295, columns AH and AI.

In some embodiments, the DruF polypeptide comprises an amino acid sequence encoded by a nucleic acid sequence having at least 80%, at least 85%, at least 90%, or at least 95% homology to a sequence selected from the group consisting of the polynucleotides referenced in Table 10, rows 124-295, columns AH and AJ. In some embodiments, the DruF polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 124-295, columns AH and AJ. In some embodiments, the DruF polypeptide comprises an amino acid sequence encoded by a nucleic acid sequence having at least 80%, at least 85%, at least 90%, or at least 95% identity to a sequence selected from the group consisting of the polynucleotides referenced in Table 10, rows 124-295, columns AH and AJ. In some embodiments, the DruF polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 124-295, columns AH and AJ. In some embodiments, the DruF polypeptide comprises an amino acid sequence encoded from a nucleic acid sequence selected from the group consisting of the polynucleotides referenced in Table 10, rows 124-295, columns AH and AJ.

In some embodiments, the nucleic acid sequence of a druF gene comprises a nucleic acid sequence having at least 80%, at least 85%, at least 90%, or at least 95% homology to a sequence selected from the group consisting of the polynucleotides referenced in Table 10, rows 124-295, columns AH and AJ. In some embodiments, the druF gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 10, rows 124-295, columns AH and AJ. In some embodiments, the nucleic acid sequence of a druF gene comprises a nucleic acid sequence having at least 80%, at least 85%, at least 90%, or at least 95% identity to a sequence selected from the group consisting of the polynucleotides referenced in Table 10, rows 124-295, columns AH and AJ. In some embodiments, the druF gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 10, rows 124-295, columns AH and AJ. In some embodiments, the nucleic acid sequence of a druF gene comprises a nucleic acid sequence selected from the group consisting of the polynucleotides referenced in Table 10, rows 124-295, columns AH and AJ.

In some embodiments, the term “DruG” refers to the polynucleotide or expression product e.g., the polypeptide encoded by the druG gene. In some embodiments, the term “DruG” refers to a DruG polypeptide. In some embodiments, the druG gene encodes a polypeptide.

In some embodiments, DruG polypeptide is encoded by a gene positioned within 5-60 genes of a gene encoding a DruM polypeptide, a DruF polypeptide, and or a DruE polypeptide in a genome of a prokaryotic cell. In some embodiments, DruG polypeptide is encoded by a gene positioned within 5 genes upstream (5′) or downstream (3′) to a gene encoding a DruM polypeptide, a DruF polypeptide, and or a DruE polypeptide in a genome of a prokaryotic cell. In some embodiments, DruG polypeptide is encoded by a gene positioned within 5 genes upstream (5′) and downstream (3′) to a gene encoding DruM polypeptide, a DruF polypeptide, and or a DruE polypeptide in a genome of a prokaryotic cell.

In some embodiments, DruG and DruE are encoded by genes positioned sequentially 5′ to 3′ in a genome of a prokaryotic cell. In some embodiments, DruG and DruE are encoded by genes positioned contiguously 5′ to 3′ in a genome of a prokaryotic cell.

In some embodiments, druG and druE genes are positioned sequentially 5′ to 3′ in a genome of a prokaryotic cell. In some embodiments, druG, and druE genes are positioned contiguously 5′ to 3′ in a genome of a prokaryotic cell.

In some embodiments, a DruG polypeptide is about 711 amino acids long (median gene size).

In some embodiments, the DruG polypeptide comprises the amino acid sequence having at least 80% homology to a sequence selected from the group consisting of the polypeptides referenced in Table 10, rows 124-295, columns AL and AM. In some embodiments, the DruG polypeptide comprises the amino acid sequence having at least 80% homology within similar domain regions within a sequence selected from the group consisting of the polypeptides referenced in Table 10, rows 124-295, columns AL and AM. In some embodiments, the DruG polypeptide comprises a homolog of a polypeptide having the amino acid sequences set forth in the polypeptides referenced in Table 10, rows 124-295, columns AL and AM.

In some embodiments, the DruG polypeptide comprises the amino acid sequence having at least 80% identity to a sequence selected from the group consisting of the polypeptides referenced in Table 10, rows 124-295, columns AL and AM. In some embodiments, the DruG polypeptide comprises the amino acid sequence having at least 80% identity within similar domain regions within a sequence selected from the group consisting of the polypeptides referenced in Table 10, rows 124-295, columns AL and AM.

In some embodiments, the DruG polypeptide comprises an amino acid sequence having at least 80%, at least 85%, at least 90%, or at least 95% homology to a sequence selected from the group consisting of the polypeptides referenced in Table 10, rows 124-295, columns AL and AM. In some embodiments, the DruG polypeptide comprises the amino acid sequence having at least 80%, 85%, 90%, 95% homology within similar domain regions within a sequence selected from the group consisting of the polypeptides referenced in Table 10, rows 124-295, columns AL and AM. In some embodiments, the DruG polypeptide comprises an amino acid sequence having at least 80%, at least 85%, at least 90%, or at least 95% identity to a sequence selected from the group consisting of the polypeptides referenced in Table 10, rows 124-295, columns AL and AM. In some embodiments, the DruG polypeptide comprises the amino acid sequence having at least 80%, 85%, 90%, 95% identity within similar domain regions within a sequence selected from the group consisting of the polypeptides referenced in Table 10, rows 124-295, columns AL and AM. In some embodiments, the DruG polypeptide comprises an amino acid sequence selected from the group consisting of the polypeptides referenced in Table 10, rows 124-295, columns AL and AM.

In some embodiments, the DruG polypeptide comprises an amino acid sequence encoded by a nucleic acid sequence having at least 80%, at least 85%, at least 90%, or at least 95% homology to a sequence selected from the group consisting of the polynucleotides referenced in Table 10, rows 124-295, columns AL and AN. In some embodiments, the DruG polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 124-295, columns AL and AN. In some embodiments, the DruG polypeptide comprises an amino acid sequence encoded by a nucleic acid sequence having at least 80%, at least 85%, at least 90%, or at least 95% identity to a sequence selected from the group consisting of the polynucleotides referenced in Table 10, rows 124-295, columns AL and AN. In some embodiments, the DruG polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 124-295, columns AL and AN. In some embodiments, the DruG polypeptide comprises an amino acid sequence encoded from a nucleic acid sequence selected from the group consisting of the polynucleotides referenced in Table 10, rows 124-295, columns AL and AN.

In some embodiments, the nucleic acid sequence of a druG gene comprises a nucleic acid sequence having at least 80%, at least 85%, at least 90%, or at least 95% homology to a sequence selected from the group consisting of the polynucleotides referenced in Table 10, rows 124-295, columns AL and AN. In some embodiments, the druG gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 10, rows 124-295, columns AL and AN. In some embodiments, the nucleic acid sequence of a druG gene comprises a nucleic acid sequence having at least 80%, at least 85%, at least 90%, or at least 95% identity to a sequence selected from the group consisting of the polynucleotides referenced in Table 10, rows 124-295, columns AL and AN. In some embodiments, the druG gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 10, rows 124-295, columns AL and AN. In some embodiments, the nucleic acid sequence of a druG gene comprises a nucleic acid sequence selected from the group consisting of the polynucleotides referenced in Table 10, rows 124-295, columns AL and AN.

In some embodiments, the term “DruH” refers to the polynucleotide or expression product e.g., the polypeptide encoded by the druH gene. In some embodiments, the term “DruH” refers to a DruH polypeptide. In some embodiments, the druH gene encodes a polypeptide.

In some embodiments, DruH polypeptide is encoded by a gene positioned within 5-60 genes of a gene encoding a DruE polypeptide in a genome of a prokaryotic cell. In some embodiments, DruH polypeptide is encoded by a gene positioned within 5 genes upstream (5′) or downstream (3′) to a gene encoding a DruE polypeptide in a genome of a prokaryotic cell. In some embodiments, DruH polypeptide is encoded by a gene positioned within 5 genes upstream (5′) and downstream (3′) to a gene encoding a DruE polypeptide in a genome of a prokaryotic cell.

In some embodiments, DruH and DruE are encoded by genes positioned sequentially 5′ to 3′ in a genome of a prokaryotic cell. In some embodiments, DruH and DruE are encoded by genes positioned contiguously 5′ to 3′ in a genome of a prokaryotic cell.

In some embodiments, druH and druE genes are positioned sequentially 5′ to 3′ in a genome of a prokaryotic cell. In some embodiments, druH, and druE genes are positioned contiguously 5′ to 3′ in a genome of a prokaryotic cell.

In some embodiments, a DruH polypeptide is about 1079 amino acids long (median gene size).

In some embodiments, the DruH polypeptide comprises the amino acid sequence having at least 80% homology to a sequence selected from the group consisting of the polypeptides referenced in Table 10, rows 296-1343, columns AP and AQ. In some embodiments, the DruH polypeptide comprises the amino acid sequence having at least 80% homology within similar domain regions within a sequence selected from the group consisting of the polypeptides referenced in Table 10, rows 296-1343, columns AP and AQ. In some embodiments, the DruH polypeptide comprises a homolog of a polypeptide having the amino acid sequences set forth in the polypeptides referenced in Table 10, rows 296-1343, columns AP and AQ.

In some embodiments, the DruH polypeptide comprises the amino acid sequence having at least 80% identity to a sequence selected from the group consisting of the polypeptides referenced in Table 10, rows 296-1343, columns AP and AQ. In some embodiments, the DruH polypeptide comprises the amino acid sequence having at least 80% identity within similar domain regions within a sequence selected from the group consisting of the polypeptides referenced in Table 10, rows 296-1343, columns AP and AQ.

In some embodiments, the DruH polypeptide comprises an amino acid sequence having at least 80%, at least 85%, at least 90%, or at least 95% homology to a sequence selected from the group consisting of the polypeptides referenced in Table 10, rows 296-1343, columns AP and AQ. In some embodiments, the DruH polypeptide comprises the amino acid sequence having at least 80%, 85%, 90%, 95% homology within similar domain regions within a sequence selected from the group consisting of the polypeptides referenced in Table 10, rows 296-1343, columns AP and AQ. In some embodiments, the DruH polypeptide comprises an amino acid sequence having at least 80%, at least 85%, at least 90%, or at least 95% identity to a sequence selected from the group consisting of the polypeptides referenced in Table 10, rows 296-1343, columns AP and AQ. In some embodiments, the DruH polypeptide comprises the amino acid sequence having at least 80%, 85%, 90%, 95% identity within similar domain regions within a sequence selected from the group consisting of the polypeptides referenced in Table 10, rows 296-1343, columns AP and AQ. In some embodiments, the DruH polypeptide comprises an amino acid sequence selected from the group consisting of the polypeptides referenced in Table 10, rows 296-1343, columns AP and AQ.

In some embodiments, the DruH polypeptide comprises an amino acid sequence encoded by a nucleic acid sequence having at least 80%, at least 85%, at least 90%, or at least 95% homology to a sequence selected from the group consisting of the polynucleotides referenced in Table 10, rows 296-1343, columns AP and AR. In some embodiments, the DruH polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 296-1343, columns AP and AR. In some embodiments, the DruH polypeptide comprises an amino acid sequence encoded by a nucleic acid sequence having at least 80%, at least 85%, at least 90%, or at least 95% identity to a sequence selected from the group consisting of the polynucleotides referenced in Table 10, rows 296-1343, columns AP and AR. In some embodiments, the DruH polypeptide comprises the amino acid sequence encoded by a nucleic acid sequence having at least 80%, 85%, 90%, 95% homology within similar domain regions within a sequence selected from the group consisting of the polynucleotides referenced in Table 10, rows 296-1343, columns AP and AR. In some embodiments, the DruH polypeptide comprises an amino acid sequence encoded from a nucleic acid sequence selected from the group consisting of the polynucleotides referenced in Table 10, rows 296-1343, columns AP and AR.

In some embodiments, the nucleic acid sequence of a druH gene comprises a nucleic acid sequence having at least 80%, at least 85%, at least 90%, or at least 95% homology to a sequence selected from the group consisting of the polynucleotides referenced in Table 10, rows 296-1343, columns AP and AR. In some embodiments, the druH gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 10, rows 296-1343, columns AP and AR. In some embodiments, the nucleic acid sequence of a druH gene comprises a nucleic acid sequence having at least 80%, at least 85%, at least 90%, or at least 95% identity to a sequence selected from the group consisting of the polynucleotides referenced in Table 10, rows 296-1343, columns AP and AR. In some embodiments, the druH gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 10, rows 296-1343, columns AP and AR. In some embodiments, the nucleic acid sequence of a druH gene comprises a nucleic acid sequence selected from the group consisting of the polynucleotides referenced in Table 10, rows 296-1343, columns AP and AR.

A skilled artisan would appreciate that the terms a “functional portion of a Defense System IIIa component” or “functional fragment of Defense System IIIa component” or “functional portion of a Druantia Type I defense system component” or “functional fragment of Druantia Type I defense system component” refers to a functional portion of a Druantia polynucleotide or polypeptide, as disclosed herein, which expression is sufficient to elicit an anti-phage activity alone or in combination with the at least one of the other Druantia polynucleotides or polypeptides disclosed herein or functional portions thereof.

A skilled artisan would appreciate that the terms a “functional portion of a Defense System IIIb component” or “functional fragment of Defense System IIIb component” or “functional portion of a Druantia Type II defense system component” or “functional fragment of Druantia Type II defense system component” refers to a functional portion of a Druantia polynucleotide or polypeptide, as disclosed herein, which expression is sufficient to elicit an anti-phage activity alone or in combination with the at least one of the other Druantia polynucleotides or polypeptides disclosed herein or functional portions thereof.

A skilled artisan would appreciate that the terms a “functional portion of a Defense System IIIc component” or “functional fragment of Defense System IIIc component” or “functional portion of a Druantia Type III defense system component” or “functional fragment of Druantia Type III defense system component” refers to a functional portion of a Druantia polynucleotide or polypeptide, as disclosed herein, which expression is sufficient to elicit an anti-phage activity alone or in combination with the at least one of the other Druantia polynucleotides or polypeptides disclosed herein or functional portions thereof.

The terms “DruA”, “DruB”, “DruC”, “DruD”, “DruE”, “DruF”, “DruG”, “DruH”, “DruM”, “druA”, “druB”, “druC”, “druD”, “druE”, “druF”, “druG”, “druH”, and “druM”, also refer to functional DruA, DruB, DruC, DruD, DruE, DruF, DruG, DruH, DruM, druA, druB, druG, druD, druE, druF, druG, druH, and druM, homologs, which exhibit the desired activity (i.e., conferring phage resistance). Such homologs can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide referenced in Table 10 rows 2-123, columns J and K; rows 2-123, columns N and O; rows 2-123, columns R and S; rows 2-123, columns V and W; rows 2-1343, columns Z and AA; rows 124-295, columns AD and AE; rows 124-295, columns AH and AI; rows 124-295, columns AL and AM, and rows 296-1343, columns AP and AQ, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% homologous or identical to the polynucleotide sequences referenced in Table 10 rows 2-123, columns J and L; rows 2-123, columns N and P; rows 2-123, columns R and T; rows 2-123, columns V and X; rows 2-1343, columns Z and AB; rows 124-295, columns AD and AF; rows 124-295, columns AH and AJ; rows 124-295, columns AL and AN, and rows 296-1343, columns AP and AR, respectively. In some embodiments, such homologs comprise at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% homology within similar domain regions of the polypeptide sequences referenced in Table 10 rows 2-123, columns J and K; rows 2-123, columns N and O; rows 2-123, columns R and S; rows 2-123, columns V and W; rows 2-1343, columns Z and AA; rows 124-295, columns ad and AE; rows 124-295, columns AH and AI; rows 124-295, columns AL and AN, and rows 296-1343, columns AP and AQ, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity within nucleotide sequences encoding similar domain regions to the polynucleotide sequences referenced in Table 10 rows 2-123, columns J and L; rows 2-123, columns N and P; rows 2-123, columns R and T; rows 2-123, columns V and X; rows 2-1343, columns Z and AB; rows 124-295, columns AD and AF; rows 124-295, columns AH and AJ; rows 124-295, columns AL and AN, and rows 296-1343, column AP and AR, respectively.

Table 10 presents embodiments of components of Defense System III (IIIa; IIIb; and IIIc) that may be found in a diverse array of bacteria and archaea genomes (referenced in Table 18). Table 10 presents embodiments of nucleic acid sequences encoding gene cassettes of Defense System III (IIIa; IIIb; and IIIc; referenced in Table 18).

The Hachiman Defense System

In some embodiments, a defense system disclosed herein comprises a Hachiman anti-phage defense system. (Table 12) In some embodiments, a Hachiman defense system provides a host cell with resistance to at least one phage.

In some embodiments, a Hachiman defense system provides a host cell with resistance to entry of foreign nucleic acid invasion. In some embodiments, a host cell expressing a functional Hachiman defense system (Defense System IV) provides the host cell resistance foreign nucleic acid invasion.

In some embodiments, a bacterial cell expressing a Hachiman defense system (Defense System IV) protects the bacteria from phage infection. In some embodiments, a bacterial cell expressing a functional Hachiman defense system (Defense System IV) protects the bacteria from phage infection.

In some embodiments, a Hachiman defense system provides a host cell with resistance to plasmid transformation. In some embodiments, a host cell expressing a functional Hachiman defense system (Defense System IV) provides a host cells with resistance to plasmid transformation.

In some embodiments, a Hachiman defense system provides a host cell with resistance to entry of conjugative elements. In some embodiments, a host cell expressing a function Hachiman defense system (Defense System IV) provides the host cell resistance from entry of conjugative elements.

As used herein, the term “a Hachiman anti-phage defense system” may be used interchangeably with the term “a Defense System IV”, having all the same meanings and qualities. A skilled artisan would appreciate that the term “Hachiman system” may be used interchangeably in some embodiments with “Hachiman defense system”, “Hachiman the defense system”, “Hachiman anti-phage system”, and “Defense System IV”, having all the same meanings and qualities.

In some embodiments, a microbial species does not comprise an endogenous Defense System IV. In some embodiments, a microbial species does not express an endogenous Defense System IV. In some embodiments, a microbial species does not express a functional Defense System IV.

In some embodiments, a bacterial species does not comprise an endogenous Defense System IV. In some embodiments, a bacterial species does not express an endogenous Defense System IV. In some embodiments, a bacterial species does not endogenously express a functional Defense System IV.

A Hachiman defense system comprises, in some embodiments, a nucleic acid construct comprising a nucleic acid sequence encoding a HamA polypeptide comprising a pfam08878 domain or a DUF1837 domain or a combination thereof, and a HamB polypeptide comprising COG1204 domain or a pfam00270 domain or a pfam00271 domain or any combination thereof. In some embodiments, a Hachiman defense system comprises a nucleic acid construct comprising a nucleic acid sequence comprising a hamA gene and a hamB gene.

A Hachiman defense system comprises, in some embodiments, a nucleic acid construct comprising a nucleic acid sequence encoding a HamA polypeptide comprising a pfam08878 or a DUF1837 domain or a combination thereof, and a HamB polypeptide comprising a COG1204 domain or a pfam00270 domain or a pfam00271 domain, or any combination thereof. In some embodiments, a Hachiman defense system comprises a nucleic acid construct comprising a nucleic acid sequence encoding a HamA polypeptide and a HamB polypeptide.

In some embodiments, a Hachiman defense system comprises, a HamA polypeptide comprising a pfam08878 or a DUF1837 domain or a combination thereof, and a HamB polypeptide comprising a COG1204 domain or a pfam00270 domain or a pfam00271 domain or any combination thereof. In some embodiments, a Hachiman defense system comprises a HamA polypeptide and a HamB polypeptide.

In some embodiments, a Defense System IV comprising a HamA polypeptide comprising a pfam08878 domain or a DUF1837 domain or any combination thereof, or comprising an amino acid sequence having at least 80% identity to a sequence selected from the group referenced in Table 12 rows 2-1782 columns H and I; or a HamB polypeptide comprising COG1204 domain or a pfam00270 domain or a pfam00271 domain or any combination thereof, or comprising an amino acid sequence having at least 80% identity to a sequence selected from the group referenced in Table 12 rows 2-1782 columns L and M; any combination thereof.

In some embodiments, a Defense System IV comprises at least two different polypeptide components selected from a HamA polypeptide comprising a pfam08878 domain or a DUF1837 domain or any combination thereof, or comprising an amino acid sequence having at least 80% identity to a sequence selected from the group referenced in Table 12 rows 2-1782 columns H and I; and a HamB polypeptide comprising COG1204 domain or a pfam00270 domain or a pfam00271 domain or any combination thereof, or comprising an amino acid sequence having at least 80% identity to a sequence selected from the group referenced in Table 12 rows 2-1782 columns L and M.

In some embodiments, a Hachiman defense system having an anti-phage activity comprises a nucleic acid construct comprising a nucleic acid sequence encoding a HamA polypeptide and a HamB polypeptide. In some embodiments, a Hachiman defense system having an anti-phage activity comprises a nucleic acid construct comprising a nucleic acid sequence comprising a hamA gene and a hamB gene.

In some embodiments, the Hachiman anti-phage defense system comprises a nucleic acid construct comprising nucleic acid sequences encoding a cassette comprising hamA and hamB genes.

In some embodiments, a construct comprising the Hachiman defense system encodes one component of the defense system, whereby multiple constructs, for example but not limited to, two constructs may be used to assemble the functional defense system. In some embodiments, the components of a Hachiman defense system comprise genes hamA and hamB. In some embodiments, the components of a Hachiman defense system consist of genes hamA and hamB. In some embodiments, the components of a Hachiman defense system comprise nucleic acid sequences encoding a HamA polypeptide and a HamB polypeptide. In some embodiments, the components of a Hachiman defense system consist of nucleic acid sequences encoding a HamA polypeptide and a HamB polypeptide.

In some embodiments, a construct comprising the Hachiman defense system encodes one component of the defense system, whereby a second construct may be used to assemble the functional defense system. In some embodiments, a construct comprising the Hachiman defense system encodes one component of the defense system, whereby additional constructs may be used to assemble the functional defense system. For example, in some embodiments each of HamA and HamB may be encoded by nucleic acid sequences comprised in different constructs, wherein expression of the combination of constructs produces a functional Hachiman defense system.

In some embodiments, the components making up a functional Hachiman anti-phage defense system comprise a HamA polypeptide and a HamB polypeptide, each encoded by a hamA and a hamB gene, respectively.

In some embodiments, a Hachiman defense system having an anti-phage activity comprises a nucleic acid encoding a HamA polypeptide. In some embodiments, a Hachiman defense system having an anti-phage activity comprises a nucleic acid encoding a HamA polypeptide and a HamB polypeptide. In some embodiments, a Hachiman defense system having an anti-phage activity comprises a nucleic acid encoding a HamB polypeptide.

In some embodiments, a Hachiman defense system having an anti-phage activity comprises a nucleic acid comprising a hamA gene. In some embodiments, a Hachiman defense system having an anti-phage activity comprises a nucleic acid comprising a hamA gene and a hamB gene. In some embodiments, a Hachiman defense system having an anti-phage activity comprises a nucleic acid comprising hamB gene.

In some embodiments, a Hachiman defense system having an anti-phage activity comprises a nucleic acid construct comprising nucleic acids encoding polypeptides in a set order. In one embodiment, the 5′ to 3′ order of polypeptides encoded is HamA and HamB. In some embodiments the 5′ to 3′ order of polypeptides does not affect the anti-phage activity. In some embodiments the 5′ to 3′ order of polypeptides does affect the anti-phage activity.

In some embodiments, the 5′ to 3′ order of polypeptides is random, for example any order of nucleic acid sequences encoding HamA and HamB.

In some embodiments, a Hachiman defense system having an anti-phage activity comprises a nucleic acid construct comprising genes in a set order. In some embodiment, the 5′ to 3′ order of genes is hamA and hamB. In some embodiment, the 5′ to 3′ order of genes in a Hachiman defense system is not hamA and hamB. In some embodiments the 5′ to 3′ order of genes does not affect the anti-phage activity. In some embodiments the 5′ to 3′ order of genes does affect the anti-phage activity.

In some embodiments, the 5′ to 3′ order of genes is random, for example any order of hamA and hamB.

In some embodiments, the Hachiman system (Defense System IV) composition and order is as shown in FIG. 3B.

In some embodiments, a Hachiman defense system having an anti-phage activity originates from a microbial genome, for example a bacterial and or an archaeal genome (Table 12). A skilled artisan would appreciate that the Hachiman system is not present in the majority of bacteria and or archaea species.

In some embodiments, introducing a combination of polypeptide components within a defense system disclosed herein, for example taking HamA from one bacterium and HamB from another bacterium and combining them into HamA-HamB functional defense system that defends against foreign elements that are not naturally occurring is advantageous to the host cell comprising the non-naturally occurring polypeptide components. In some embodiments, introducing a combination of polypeptide components within any defense system disclosed herein, and combining them to form a functional defense system that defends against foreign elements that are not naturally occurring, is advantageous to the host cell comprising the combination of non-naturally occurring polypeptide components.

In some embodiments, a functional Hachiman defense system comprises a construct comprising nucleic acid sequences encoding polypeptides, wherein the nucleic acid sequence encoding each polypeptide may originate from a different microbial species. For example, the source of the nucleic acid sequence encoding a HamA polypeptide may be one microbial species, while the source of the nucleic acid sequence encoding a HamB polypeptide may be a different microbial species. In some embodiments, a functional Hachiman defense system comprises a non-naturally occurring combination of polypeptide components. In some embodiments, a functional Hachiman defense system comprises a combination of at least two polypeptides that do not naturally occur together.

In some embodiments, a functional Hachiman defense system comprises a construct comprising nucleic acid sequences encoding polypeptides, wherein the nucleic acid sequence encoding each polypeptide may originate from a different bacterial species. For example, the source of the nucleic acid sequence encoding a HamA polypeptide may be one bacterial species, while the source of the nucleic acid sequence encoding a HamB polypeptide may be a different bacterial species.

In some embodiments, the source of the nucleic acid encoding a HamA polypeptide and a HamB polypeptide is the same. In some embodiments, the source of the nucleic acid encoding a HamA polypeptide and a HamB polypeptide is the not the same. In some embodiments, the source of the nucleic acid sequence of any of the components of a Hachiman defense system comprises any of the species listed in Table 12.

In some embodiments, a Hachiman defense system having an anti-phage activity comprises a nucleic acid construct comprising non-coding regions between the nucleic acid sequences encoding the polypeptides. In some embodiments, the non-coding regions between the nucleic acid sequences comprises nucleic acid sequence not naturally occurring in the microbial species of origin. In some embodiments, the non-coding regions between the nucleic acid sequences comprises nucleic acid sequence is the naturally occurring in the microbial species of origin.

In some embodiments, a Hachiman system disclosed herein comprises a multi-gene phage resistance system broadly distributed in microbial genomes, for example but not limited to bacteria and archaea. According to some embodiments, the Hachiman system components are located in a gene cluster (a cassette of genes) in a microbial cell genome. According to some embodiments, the Hachiman system components are located in close proximity (e.g. positioned within 20 genes, 10 genes, 5 genes or less one from the other) in a genome of a prokaryotic cell. According to some embodiments the prokaryotic cell expresses an endogenous Hachiman defense system. According to some embodiments, the prokaryotic cell expresses an endogenous functional Hachiman defense system. According to some embodiments, the species of prokaryotic cell is selected from the group consisting of the species listed in Table 12. According to some embodiments a prokaryotic cell expresses a non-endogenous Hachiman defense system. According to some embodiments, a prokaryotic cell expresses a non-endogenous functional Hachiman defense system. According to some embodiments, the species of prokaryotic cell expressing a non-endogenous functional Hachiman defense system is selected from the group consisting of the species listed in Table 12.

In some embodiments, a Hachiman (Defense System IV) gene cassette comprises the nucleic acid sequence:

(SEQ ID NO: 4; construct 3 Table 4)

| | |
|---|---|
| 1 | taaaagctgc ttcaggtaaa tatcctgaag cagcttttgt ggttaaaaga gaaagtaggt |
| 61 | aatagtcatt tgtagtgtat taaagaagat ggaggtgaat aaaacgatgt aatttaagcc |
| 121 | ataagttggc cgctttggcc aatgttttct ttattttata tatggtaaaa ttataattag |
| 181 | tttagatagt acatttgctt agtaaagatg tactttaaaa aaaaataaat ttatttttgt |
| 241 | ggctcttaac gaaatgaata tgctattttg atgtatctta ggtagaaatc taaaaatatt |
| 301 | aagaagttgt taaacagcgt taagctagga ggaaatatat gactatacaa gatgatatgg |
| 361 | ttggaaaaca tcctatcgag aatgattttt ggaaatggtt gcagcatgaa gatacagaat |
| 421 | cctcggattt aaaacggcat cgttacttag aagtggattc tagtaatcgt gatgaagcta |
| 481 | ttaaatctgt tgcagcttgg ctaattaagt atcatttgtc ggaaggtaaa aagagagtga |
| 541 | taagaaaaaa acaagaaata cttgaaaaac atgattttgc tgaatatgca caatcgcttc |
| 601 | atgtttttcc taaaagtgat aaaacacaga aaggaaattt aggagaaata tttttatcgg |
| 661 | aatatttgag tcagacatca ggagttcaaa tcctagtgta taaattacac tacaacccca |
| 721 | acatagatca atcgatgaaa ggggatgacg ttttacttgt taacccagat aaagttttat |
| 781 | taggagaaag taaatttcgt tctactccaa ataaacgtgc tgttgaagag gcttctgaac |
| 841 | ttatgaaaga taagttaact ttgcctatgt ctcttggttt tattgcagat agattatttg |
| 901 | aacaaggaaa agatgaatta ggtgaagtga ttttcgatct tcaatttaag atgagtagta |
| 961 | tagaaataga tattaaaaat atagggtttt tattatctac taaaaaggtt cgtagtatcg |
| 1021 | tagaaaataa cctttcttct ccaaattctg attttatttt tatttcatta ggaatggatg |
| 1081 | acccagcagc ttttttgaaa agtgtatttg actatgcaga aagtaatctt ctggagggat |
| 1141 | cttatgaaac ttgaaacagc atataattat tttgaaaagc ttgaaaaaga tgaacgtctt |
| 1201 | caaaactata tttcacaggc taattcaaga tatatccttt acatagcaaa tgaaccattg |
| 1261 | gaaaatttcc cacattacac tgtaaatcta gatgaaaaat gtacacatat agcatttagt |
| 1321 | tatctcaatt gtggatggcg ctttttttta gagaaaatca gtgatgaagc tacaaaatgt |
| 1381 | atggaaaagg cttctgaaat attggagtat ttgtatgcct atagtgattg cgaaaagtta |
| 1441 | tataaagagt attaccgttt agtatgctca ttagcgtatt atatatctgc acagtattct |
| 1501 | aaatctttta ttatcttggg gaaatataat tctgattcat atatagggaa aataacaaaa |
| 1561 | ttatttttaa ctagaaactt tatagaatta gaaaggcaac tggaaattaa gtacttgaag |
| 1621 | aacgatgaag aaacttcaag tgagaataag gataacataa tatatgaaaa aattttatct |
| 1681 | aatgcatttt tacatataat taattttatt cgcacaggga agatggaatc tttggaaagt |
| 1741 | tctaaagata tggttcgaga tttaattact ttggctgata taaataatga accacatatg |
| 1801 | tggtggtact ttagactact atatttggtt tttgaagaat atgaagatgg gtctttgtgg |
| 1861 | aaagtggttc caccgctttt agataatgat gatatatcag ctaaatatat ttatgctaat |
| 1921 | ttatacaaaa agaagccaat aactgaattt tttaagtcgc aaagggaatg tttggacgga |
| 1981 | gagttattaa agaacgatgg tttcgtaatt ggaatgccta ctagttcagg aaaaacaaag |
| 2041 | gttgctgaag taacaatatt aaaaacttta actaaatctc caggtgcact ttgtatttat |
| 2101 | attgcacctt atcgatcttt ggctaatgaa gtggaattta gcctatctag tatgtttgag |
| 2161 | attatgggtt atcaggtttc gcagttatat gggggatctc aaacgtctcc gctggatcgg |
| 2221 | caattgacta agcaagctaa tgtcataatt gttactccgg aaaaagctaa atcgatgttg |
| 2281 | agatctaata aagacttaaa ggatagaatt caacttgtaa tcattgatga aggtcattta |
| 2341 | gtcgggtttc agccaagata tattacagga gaattgctta ttgaagaact aaagattgtt |
| 2401 | ttgaaaaaaa ataatggtca acttgtacta ctatctgctg ttcttcctaa tatttctgat |
| 2461 | ttttctttgt ggattggtga ggatgataaa gctaaaagaa tgtctgtctg gagaccatca |
| 2521 | agtcaaagat ttggagaatt atctttagca agaaatactg tgaatataaa ttgggaggga |
| 2581 | gaaacaccat catataataa gaactttata acaccaaaat tagtaaaacc agaacggatt |
| 2641 | acaaaaacag gaaggaaata tgcagctaaa tattttccag cagataaaaa agaaggtgtt |
| 2701 | gctgcaaccg caacaaaaat gctagatgtg ggatccgttt tgatttatgt aggaagatcg |
| 2761 | aatatggtat tgtctcaagc taggattgtt tcgaaactct ttgaggaaaa gggaatagaa |
| 2821 | catgagtgga ggaataaaaa cgattggttg tttgtggaat tagcttgtat agaagaatat |
| 2881 | ggggaagatt cagaaatatt atctttatta aaacaaggta tagtacccca tagttctaaa |
| 2941 | ttaccaacag aagtacgtca gagcatagag aaattaatgg ctaatgataa tcctaagata |
| 3001 | ataattgcga catccacact tggacaaggg gttaatattg gtgtttcgac agttattgta |
| 3061 | tctaatgttc gtttagatga gagtaatact gtaaaagtaa atgatttttg gaatattgct |
| 3121 | ggtagagcgg ggagggcatt tacagataca gagggtaaaa ttctatacgc aattgataga |
| 3181 | aataaaagcc agtggtctat ttataatcaa attcaaatga aagaaatgta tttcaaatat |
| 3241 | agaaatattg aaaaagctac aagtggtctc tatcttcttc ttcgttatct atttatatta |
| 3301 | tcggaagaat tcgagataga atacagctta tttttagaac ttctagcaga aaatagagaa |
| 3361 | tccattgatg aacagaaagc tgtggagttt ttttctgagg cagatcagtt ccttgagtta |
| 3421 | ttagatgata ctctaatttc catggatatt atgaatgagg caaacgtttt ggaaaattcc |
| 3481 | tcttcttgga tagacgatgt ctttcgttct tcgctagctt ttattcaagc aaaacaagat |
| 3541 | caatcatttt ctgaagaaaa aatgattgaa atcttaaaag caagaaattc aggtgtaata |
| 3601 | aagttggctg gagaagaaac aaaatggcat tcaatagcat catcaagtgt accattgaga |
| 3661 | gcaagtcttg taattgaaca taggatagat gagctactaa agtatgtacg aaaatacatt |
| 3721 | gactcagata agagttttga agagttatta tctcttgtga tggaacttga tatatttatt |
| 3781 | gatagtctac cgatatctaa aatagaagga atagaaatag gtgctaattt ttctgaaatt |
| 3841 | cgtgagggat ggtttagtgg tactagcata cacatattgg aaaaaaggca cgaaaatgta |
| 3901 | agaagtatct gtaatgaata ttattctttt cattttcctt ggattgtaaa tgcgatagct |
| 3961 | caaaagatca aattaattga ttgccaagag gaatcaaagg tgttggagca aatttcttta |
| 4021 | tttgctgaaa tagggttaaa caataataaa agtgtaaaaa tctatttggc aggtataaag |
| 4081 | tcaagaaatt gtgccactga attatctgag aaaataggca tggataatga tttggtgacg |
| 4141 | cctgttaagg agctacttct agagtttttc aatttatgtg tagtgaaaaa agaggattgt |
| 4201 | tcagaaattt gtttcaattg gttaaagctt ttaaatgatg aaaaagaagc taaagagtat |
| 4261 | gtaaaggtaa aaagacggtc tttccaattt aatattcctt ttgatttaca tgacaatctt |
| 4321 | ttatatataa agaaagtaca gagtgaaata tggctgtgct catttgatta taaaattaaa |
| 4381 | attcctgtat taaaagagaa ggcgttattt aatgagtgct cggatatacc tgatattttc |
| 4441 | ttgagaaagg aaaaaggaga tgtctggatt ttagattctc ataatcctta tattattttt |
| 4501 | tattaattta tttgaattta atattttgga taaggcagat tgttcaacta tttggaggaa |
| 4561 | ataaaaaaac ggattattaa gggtgaacta aatttaatca aactttttac aaatctgcat |
| 4621 | ctttctcatt ttcaccttta aaaatgccaa ggaactcaat gaaatttggt atgcagatac |
| 4681 | cttcaagagc caccaatcct aaagtgaaat ctccatagtt tttacgatat tcttcataaa |
| 4741 | ttaatctggc tatatcttca ccaaaagaaa acatttcttt tgttatttcg gaaagataag |
| 4801 | gtagaagtgg tatatactca tctggtttac ctttttcatt tgaaggtact tttactgcat |
| 4861 | acccttgatc aagtgtcata atgttaattt gatgaccact atgtacaata gaatcgcgta |
| 4921 | tttctctatt cttatgaaag ctttctataa tagataatag ttttttttct aaggattcag |
| 4981 | gtaatttatc cttggttgtt cctttttcaa cggaagtaat taaactattt aaggaatcaa |
| 5041 | aactgagttg tccaacaaat ttaggagcta ctgttaatcg taggattttt gccatgaaat |
| 5101 | cataaattga acgaatatcc at. |

The coding regions for each of the hamA and hamB gene sequences within this embodiment of a Hachiman cassette (SEQ ID NO: 4) are as follows: nucleotides 339-1154 encode an embodiment of a HamA polypeptide, and nucleotides 1114-4506 encode an embodiment of a HamB polypeptide.

In some embodiments, a Defense System IV comprising a HamA polypeptide and a HamB polypeptide is encoded by the nucleic acid sequence set forth in SEQ ID NO: 4. In some embodiments, a Defense System IV comprising a HamA polypeptide and a HamB polypeptide, is encoded by a nucleic acid sequence having at least 80% homology to the sequence set forth in SEQ ID NO: 4. In some embodiments, a Defense System IV comprising a HamA polypeptide and a HamB polypeptide, is encoded by a nucleic acid sequence having at least 85% homology, or 90% homology, or 95% homology, or 97% homology, or 98% homology, or 99% homology to the sequence set forth in SEQ ID NO: 4. In some embodiments, a Defense System IV comprising a HamA polypeptide and a HamB polypeptide, is encoded by a nucleic acid sequence having at least 80% identity to the sequence set forth in SEQ ID NO: 4. In some embodiments, a Defense System IV comprising a HamA polypeptide and a HamB polypeptide, is encoded by a nucleic acid sequence having at least 85% identity, or 90% identity, or 95% identity, or 97% identity, or 98% identity, or 99% identity to the sequence set forth in SEQ ID NO: 4.

In some embodiments, Hachiman defense system components comprise HamA and HamB polypeptides. In some embodiments, Hachiman defense system components comprise functional portions of HamA and HamB polypeptides. In some embodiments, the Hachiman defense system components are encoded by hamA and hamB genes.

Non-limiting embodiments of endogenous Hachiman systems and the respective location of their components are provided in Table 12 herein.

In some embodiments, the components of a Hachiman system may be identified by the presence of similar domains present within each component. In some embodiments, domains comprise pfam domains and or clusters of orthologous groups (COG) domains.

In some embodiments, the term “HamA” refers to the polynucleotide or expression product e.g., the polypeptide encoded by the hamA gene. In some embodiments, the term “HamA” refers to a HamA polypeptide. In some embodiments, the hamA gene encodes a polypeptide comprising a pfam08878 domain. In some embodiments, the hamA gene encodes a polypeptide comprising a DUF1837 domain. In some embodiments, the hamA gene encodes a polypeptide comprising a pfam08878 domain and a DUF1837 domain. In some embodiments, the HamA polypeptide comprises a pfam08878 domain. In some embodiments, the HamA polypeptide comprises a DUF1837 domain. In some embodiments, the HamA polypeptide comprises a pfam08878 domain and a DUF1837 domain.

In some embodiments, HamA polypeptide is encoded by a gene positioned within 5-60 genes of a gene encoding a HamB polypeptide in a genome of a prokaryotic cell. In some embodiments, HamA polypeptide is encoded by a gene positioned within 5 genes upstream (5′) or downstream (3′) to a gene encoding a HamB polypeptide in a genome of a prokaryotic cell. In some embodiments, HamA polypeptide is encoded by a gene positioned within 5 genes upstream (5′) and downstream (3′) to a gene encoding a HamB polypeptide in a genome of a prokaryotic cell.

In some embodiments, HamA and HamB are encoded by genes positioned sequentially 5′ to 3′ in a genome of a prokaryotic cell. In some embodiments, HamA and HamB are encoded by genes positioned contiguously 5′ to 3′ in a genome of a prokaryotic cell.

In some embodiments, hamA and hamB genes are positioned sequentially 5′ to 3′ in a genome of a prokaryotic cell. In some embodiments, hamA and hamB, genes are positioned contiguously 5′ to 3′ in a genome of a prokaryotic cell.

In some embodiments, a HamA polypeptide is about 269 amino acids long (median gene size).

In some embodiments, the HamA polypeptide comprises the amino acid sequence having at least 80% homology to a sequence selected from the group consisting of the polypeptides referenced in Table 12, rows 2-1782, columns H and I. In some embodiments, the HamA polypeptide comprises the amino acid sequence having at least 80% homology within similar domain regions within a sequence selected from the group consisting of the polypeptides referenced in Table 12, rows 2-1782, columns H and I. In some embodiments, the HamA polypeptide comprises a homolog of a polypeptide having the amino acid sequences set forth in the polypeptides referenced in Table 12, rows 2-1782, columns H and I.

In some embodiments, the HamA polypeptide comprises the amino acid sequence having at least 80% identity to a sequence selected from the group consisting of the polypeptides referenced in Table 12, rows 2-1782, columns H and I. In some embodiments, the HamA polypeptide comprises the amino acid sequence having at least 80% identity within similar domain regions within a sequence selected from the group consisting of the polypeptides referenced in Table 12, rows 2-1782, columns H and I.

In some embodiments, the HamA polypeptide comprises an amino acid sequence having at least 80%, at least 85%, at least 90%, or at least 95% homology to a sequence selected from the group consisting of the polypeptides referenced in Table 12, rows 2-1782, columns H and I. In some embodiments, the HamA polypeptide comprises the amino acid sequence having at least 80%, 85%, 90%, 95% homology within similar domain regions within a sequence selected from the group consisting of the polypeptides referenced in Table 12, rows 2-1782, columns H and I. In some embodiments, the HamA polypeptide comprises an amino acid sequence having at least 80%, at least 85%, at least 90%, or at least 95% identity to a sequence selected from the group consisting of the polypeptides referenced in Table 12, rows 2-1782, columns H and I. In some embodiments, the HamA polypeptide comprises the amino acid sequence having at least 80%, 85%, 90%, 95% identity within similar domain regions within a sequence selected from the group consisting of the polypeptides referenced in Table 12, rows 2-1782, columns H and I. In some embodiments, the HamA polypeptide comprises an amino acid sequence selected from the group consisting of the polypeptides referenced in Table 12, rows 2-1782, columns H and I.

In some embodiments, the HamA polypeptide comprises an amino acid sequence encoded by a nucleic acid sequence having at least 80%, at least 85%, at least 90%, or at least 95% homology to a sequence selected from the group consisting of the polynucleotides referenced in Table 12, rows 2-1782, columns H and J. In some embodiments, the HamA polypeptide comprises the amino acid sequence encoded by a nucleic acid sequence having at least 80%, 85%, 90%, 95% homology within similar domain regions within a sequence selected from the group consisting of the polynucleotides referenced in Table 12, rows 2-1782, columns H and J. In some embodiments, the HamA polypeptide comprises an amino acid sequence encoded by a nucleic acid sequence having at least 80%, at least 85%, at least 90%, or at least 95% identity to a sequence selected from the group consisting of the polynucleotides referenced in Table 12, rows 2-1782, columns H and J. In some embodiments, the HamA polypeptide comprises the amino acid sequence encoded by a nucleic acid sequence having at least 80%, 85%, 90%, 95% homology within similar domain regions within a sequence selected from the group consisting of the polynucleotides referenced in Table 12, rows 2-1782, columns H and J. In some embodiments, the HamA polypeptide comprises an amino acid sequence encoded from a nucleic acid sequence selected from the group consisting of the polynucleotides referenced in Table 12, rows 2-1782, columns H and J.

In some embodiments, the nucleic acid sequence of a hamA gene comprises a nucleic acid sequence having at least 80%, at least 85%, at least 90%, or at least 95% homology to a sequence selected from the group consisting of the polynucleotides referenced in Table 12, rows 2-1782, columns H and J. In some embodiments, the hamA gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 12, rows 2-1782, columns H and J. In some embodiments, the nucleic acid sequence of a hamA gene comprises a nucleic acid sequence having at least 80%, at least 85%, at least 90%, or at least 95% identity to a sequence selected from the group consisting of the polynucleotides referenced in Table 12, rows 2-1782, columns H and J. In some embodiments, the hamA gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 12, rows 2-1782, columns H and J. In some embodiments, the nucleic acid sequence of a hamA gene comprises a nucleic acid sequence selected from the group consisting of the polynucleotides referenced in Table 12, rows 2-1782, columns H and J.

As used herein, the term “HamB” refers to the polynucleotide or expression product e.g., polypeptide encoded by the hamB gene. In some embodiments, the term “HamB” refers to a HamB polypeptide. In some embodiments, the product of the hamB gene comprises a COG1204 domain. In some embodiments, the hamB gene encodes a pfam00270 domain. In some embodiments, the hamB gene encodes a pfam00271 domain. In some embodiments, the hamB gene encodes a helicase.

In some embodiments, HamB polypeptide is encoded by a gene positioned within 5-60 genes of a gene encoding a HamA polypeptide in a genome of a prokaryotic cell. In some embodiments, HamB polypeptide is encoded by a gene positioned within 5 genes upstream (5′) or downstream (3′) to a gene encoding a HamA polypeptide in a genome of a prokaryotic cell. In some embodiments, HamB polypeptide is encoded by a gene positioned within 5 genes upstream (5′) and downstream (3′) to a gene encoding a HamA polypeptide in a genome of a prokaryotic cell.

In some embodiments, HamA and HamB are encoded by genes positioned sequentially 5′ to 3′ in a genome of a prokaryotic cell. In some embodiments HamA and HamB are encoded by genes positioned contiguously 5′ to 3′ in a genome of a prokaryotic cell.

In some embodiments, a HamB polypeptide is about 867 amino acids long (median gene size).

In some embodiments, the HamB polypeptide comprises the amino acid sequence having at least 80% homology to a sequence selected from the group consisting of the polypeptides referenced in Table 12, rows 2-1782, columns L and M. In some embodiments, the HamB polypeptide comprises the amino acid sequence having at least 80% homology within similar domain regions within a sequence selected from the group consisting of the polypeptides referenced in Table 12, rows 2-1782, columns L and M. In some embodiments, the HamB polypeptide comprises a homolog of a polypeptide having the amino acid sequences set forth in the polypeptides referenced in Table 12, rows 2-1782, columns L and M.

In some embodiments, a HamB polypeptide homolog comprises a helicase domain.

In some embodiments, a HamB polypeptide homolog comprises a helicase activity.

In some embodiments, the HamB polypeptide comprises the amino acid sequence having at least 80% identity to a sequence selected from the group consisting of the polypeptides referenced in Table 12, rows 2-1782, columns L and M. In some embodiments, the HamB polypeptide comprises the amino acid sequence having at least 80% identity within similar domain regions within a sequence selected from the group consisting of the polypeptides referenced in Table 12, rows 2-1782, columns L and M.

In some embodiments, the HamB polypeptide comprises an amino acid sequence having at least 80%, at least 85%, at least 90%, or at least 95% homology to a sequence selected from the group consisting of the polypeptides referenced in Table 12, rows 2-1782, columns L and M. In some embodiments, the HamB polypeptide comprises the amino acid sequence having at least 80%, 85%, 90%, 95% homology within similar domain regions within a sequence selected from the group consisting of the polypeptides referenced in Table 12, rows 2-1782, columns L and M. In some embodiments, the HamB polypeptide comprises an amino acid sequence having at least 80%, at least 85%, at least 90%, or at least 95% identity to a sequence selected from the group consisting of the polypeptides referenced in Table 12, rows 2-1782, columns L and M. In some embodiments, the HamB polypeptide comprises the amino acid sequence having at least 80%, 85%, 90%, 95% identity within similar domain regions within a sequence selected from the group consisting of the polypeptides referenced in Table 12, rows 2-1782, columns L and M. In some embodiments, the HamB polypeptide comprises an amino acid sequence selected from the group consisting of the polypeptides referenced in Table 12, rows 2-1782, columns L and M.

In some embodiments, the HamB polypeptide comprises an amino acid sequence encoded by a nucleic acid sequence having at least 80%, at least 85%, at least 90%, or at least 95% homology to a sequence selected from the group consisting of the polynucleotides referenced in Table 12, rows 2-1782, columns L and N. In some embodiments, the HamB polypeptide comprises the amino acid sequence encoded by a nucleic acid sequence having at least 80%, 85%, 90%, 95% homology within similar domain regions within a sequence selected from the group consisting of the polynucleotides referenced in Table 12, rows 2-1782, columns L and N. In some embodiments, the HamB polypeptide comprises an amino acid sequence encoded by a nucleic acid sequence having at least 80%, at least 85%, at least 90%, or at least 95% identity to a sequence selected from the group consisting of the polynucleotides referenced in Table 12, rows 2-1782, columns L and N. In some embodiments, the HamB polypeptide comprises the amino acid sequence encoded by a nucleic acid sequence having at least 80%, 85%, 90%, 95% homology within similar domain regions within a sequence selected from the group consisting of the polynucleotides referenced in Table 12, rows 2-1782, columns L and N. In some embodiments, the HamB polypeptide comprises an amino acid sequence encoded from a nucleic acid sequence selected from the group consisting of the polynucleotides referenced in Table 12, rows 2-1782, columns L and N.

In some embodiments, the nucleic acid sequence of a hamB gene comprises a nucleic acid sequence having at least 80%, at least 85%, at least 90%, or at least 95% homology to a sequence selected from the group consisting of the polynucleotides referenced in Table 12, rows 2-1782, columns L and N. In some embodiments, the hamB gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 12, rows 2-1782, columns L and N. In some embodiments, the nucleic acid sequence of a hamB gene comprises a nucleic acid sequence having at least 80%, at least 85%, at least 90%, or at least 95% identity to a sequence selected from the group consisting of the polynucleotides referenced in Table 12, rows 2-1782, columns L and N. In some embodiments, the hamB gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 12, rows 2-1782, columns L and N. In some embodiments, the nucleic acid sequence of a hamB gene comprises a nucleic acid sequence selected from the group consisting of the polynucleotides referenced in Table 12, rows 2-1782, columns L and N.

A skilled artisan would appreciate that the terms a “functional portion of a Defense System IV component” or “functional fragment of Defense System IV component” or “functional portion of a Hachiman defense system component” or “functional fragment of Hachiman defense system component” refers to a functional portion of a Hachiman polynucleotide or polypeptide, as disclosed herein, which expression is sufficient to elicit an anti-phage activity alone or in combination with the at least one of the other Hachiman polynucleotides or polypeptides disclosed herein or functional portions thereof.

The terms “HamA”, “HamB”, “hamA”, and “hamB”, also refer to functional HamA, HamB, hamA, and hamB, homologs, which exhibit the desired activity (i.e., conferring phage resistance). Such homologs can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide sequences referenced in Table 12 rows 2-1782 columns H and I and L and M, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% homologous or identical to the polynucleotide sequences referenced in Table 12 rows 2-1782 columns H and J and L and N, respectively. In some embodiments, such homologs comprise at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% homology or identity within similar domain regions of the polypeptide sequences referenced in Table 12 rows 2-1782 columns H and I and L and M, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% homology or identity within nucleotide sequences encoding similar domain regions to the polynucleotide sequences referenced in Table 12 rows 2-1782 columns H and J and L and N, respectively.

Table 12 presents embodiments of components of Defense System IV that may be found in a diverse array of bacteria and archaea genomes (referenced in Table 18). Table 12 presents embodiments of nucleic acid sequences encoding gene cassettes of Defense System IV (referenced in Table 18).

The Shedu Defense System

In some embodiments, a defense system disclosed herein comprises a Shedu anti-phage defense system. (Table 13)

In some embodiments, a Shedu defense system provides a host cell with resistance to entry of foreign nucleic acid invasion. In some embodiments, a host cell expressing a functional Shedu defense system (Defense System V) provides the host cell resistance foreign nucleic acid invasion.

In some embodiments, a Shedu defense system provides a host cell with resistance to at least one phage. In some embodiments, a microbial cell comprising a Shedu defense system is protected from phage infection. In some embodiments, a microbial cell expressing a Shedu defense system is protected from phage infection. In some embodiments, a microbial cell expressing a functional Shedu defense system is protected from phage infection. In some embodiments, a bacterial cell comprising a Shedu defense system is protected from phage infection. In some embodiments, a bacterial cell expressing a Shedu defense system is protected from phage infection. In some embodiments, a bacterial cell expressing a functional Shedu defense system is protected from phage infection.

In some embodiments, a Shedu defense system provides a host cell with resistance to a plasmid.

In some embodiments, a Shedu defense system (Defense System V) provides a host cell with resistance to plasmid transformation. In some embodiments, a host cell expressing a functional Shedu defense system (Defense System V) provides a host cells with resistance to plasmid transformation.

In some embodiments, a Shedu defense system (Defense system V) provides a host cell with resistance to entry of conjugative elements. In some embodiments, a host cell expressing a function Shedu defense system (Defense System V) provides the host cell resistance from entry of conjugative elements.

As used herein, the term “a Shedu anti-phage defense system” may be used interchangeably with the term “a Defense System V”, having all the same meanings and qualities. A skilled artisan would appreciate that the term “Shedu system” may be used interchangeably in some embodiments with “Shedu defense system”, “Shedu the defense system”, “Shedu anti-phage system”, and “Defense System V”, having all the same meanings and qualities.

In some embodiments, a microbial species does not comprise an endogenous Defense System V. In some embodiments, a microbial species does not express an endogenous Defense System V. In some embodiments, a microbial species does not express a functional Defense System V.

In some embodiments, a bacterial species does not comprise an endogenous Defense System V. In some embodiments, a bacterial species does not express an endogenous Defense System V. In some embodiments, a bacterial species does not express an endogenous functional Defense System V.

A Shedu defense system comprises, in some embodiments, a nucleic acid construct comprising a nucleic acid sequence encoding a SduA polypeptide comprising a pfam14082 domain or a DUF4263 domain or a pfam01939 domain, or a combination thereof. In some embodiments, a Shedu defense system comprises a SduA polypeptide comprising an endonuclease NucS activity. A skilled artisan would appreciate that an endonuclease NucS activity encompasses an enzymatic activity cleaving both 3′ and 5′ ssDNA extremities of branched DNA structures, wherein the NucS endonuclease may bind to ssDNA. In some embodiments, a Shedu defense system comprises a nucleic acid construct comprising a nucleic acid sequence comprising a sduA gene.

A Shedu defense system comprises, in some embodiments, a nucleic acid construct comprising a nucleic acid sequence comprising a sduA gene encoding a pfam14082 domain or a DUF4263 domain or a pfam01939 domain, or a combination thereof. In some embodiments, a Shedu defense system comprising a sduA gene encodes a polypeptide comprising an endonuclease NucS activity.

In some embodiments, a Defense System V comprises a SduA polypeptide comprising a pfam14082 domain or a pfam01939 domain or comprising an amino acid sequence having at least 80% identity to a sequence selected from the group referenced in Table 13 rows 2-1247 columns H and I or a combination thereof.

In some embodiments, a Shedu defense system having an anti-phage activity comprises a nucleic acid construct comprising a nucleic acid sequence encoding a SduA polypeptide. In some embodiments, a Shedu defense system having an anti-phage activity comprises a nucleic acid construct comprising a nucleic acid sequence comprising a sduA gene.

In some embodiments, the Shedu anti-phage defense system comprises a nucleic acid construct comprising nucleic acid sequences encoding a cassette comprising a sduA gene.

In some embodiments, a construct comprising the Shedu defense system encodes one component of the defense system. In some embodiments, the components of a Shedu defense system comprise a sduA gene. In some embodiments, the components of a Shedu defense system consist of a sduA gene. In some embodiments, the components of a Shedu defense system comprise nucleic acid sequences encoding a SduA polypeptide. In some embodiments, the components of a Shedu defense system consist of nucleic acid sequences encoding a SduA polypeptide.

In some embodiments, a construct comprising the Shedu defense system encodes one component of the defense system, whereby a second construct may be used to assemble the functional defense system. In some embodiments, a construct comprising the Shedu defense system encodes one component of the defense system, whereby additional constructs may be used to assemble the functional defense system. In some embodiments, the Shedu defense system comprises non-coding RNA.

In some embodiments, the components making up a functional Shedu anti-phage defense system comprise a SduA polypeptide encoded by a sduA gene.

In some embodiments, a Shedu defense system having an anti-phage activity comprises a nucleic acid encoding a SduA polypeptide.

In some embodiments, a Shedu defense system having an anti-phage activity comprises a nucleic acid comprising a sduA gene.

In some embodiments, the Shedu system (Defense System V) composition and order is as shown in FIG. 3B.

In some embodiments, a Shedu defense system having an anti-phage activity originates from a microbial genome, for example a bacterial and or an archaeal genome (Table 13). A skilled artisan would appreciate that the Shedu system is not present in the majority of bacteria and or archaea species.

In some embodiments, a Shedu defense system having an anti-phage activity comprises a nucleic acid construct comprising non-coding regions. In some embodiments, the non-coding regions comprises nucleic acid sequence not naturally occurring in the microbial species of origin. In some embodiments, the non-coding regions comprises nucleic acid sequence is the naturally occurring in the microbial species of origin.

In some embodiments, Shedu defense system components comprise functional portions of a SduA polypeptides. In some embodiments, the Shedu defense system functional portions of a SduA polypeptide component is encoded by a sduA gene.

Non-limiting embodiments of endogenous Shedu systems and the respective location of their component are provided in Table 13 herein. In some embodiments, a Shedu (Defense System V) gene cassette comprises the nucleic acid sequence:

(SEQ ID NO: 9; construct 28 Table 4)

| | |
|---|---|
| 1 | aatgaaattt gaacgccttt cggcagtgat atttgtacac ctcattttta cgtgccagcg |
| 61 | tgatgtacat gaaaagtggt gaaaagattc ctattgtggc tgagagtagt agcatgatga |
| 121 | tggtgtttct tttttctttt ttgatggttt tcatgagtga tttggccccc tttttgtttt |
| 181 | gtcttcttta aattaggggt gacgttgggg gagatgcaag gggaatctgt agaaaaattg |
| 241 | attgagaccg atatagataa attccatatt tttatttatg aaatcctata ctgtcaatat |
| 301 | taatcaaaac aattaatgag atcaaatttt tttgttcatc agacattaat agaatgattg |
| 361 | tatggataat agtagtttct ttatcacaac aacgcattat catgaagctc cacaatagga |
| 421 | ttgtagaata acctcaaatt caatcctatg ttcattgaaa agggggcggg gaactcttta |
| 481 | aacatctcca cgaggtattt tgagtgtatg tgctttatta attctacatg cttaggctaa |
| 541 | ctaatcatta atagactaca aaaaacattc atgcctagta cagaaacaca cccttctcat |
| 601 | tgaagaagac taaccagtgg tattcgcaaa agaagaaaag aggattacct caacagccac |
| 661 | ctaccttttt atattgtaaa tatgtcctat ttaattcgga gtagtgtata ctattagtag |
| 721 | aaaattccaa gcgtagctat cgaggagtgt taacaaaata tgagtgacaa aattaatgta |
| 781 | tggacaacat caagagactc tgctgtatgt ggtgacattg aattaaaaaa aacatctact |
| 841 | actagactta tttttaggcc tgaaatagtg aacaataata aaaatcctaa ggcatctgtt |
| 901 | agaggatgct ttatctttca aaagaaaggt agaaatgccc tttgggatga ttataaggaa |
| 961 | ttagatatga ataagttaaa agcagaggaa tggattaaac tagagataaa ctctgatgct |
| 1021 | atgctgactc ttactaaaga aattcagaag cattatgcag tgcatgaaaa gtatggggtt |
| 1081 | aggtatggag catttcacct tttcaaggat aaccctgata tcgagaaact gattgaaatg |
| 1141 | tttgagagta acactgattt gcttactcaa ttaatggagg atgacaagag tgaagctcta |
| 1201 | gagaaaacat tagaatggat tgttacaaat gataatccag ataaaataat tgacaggctt |
| 1261 | aaaaatctaa aagagcaaga tttagaccaa cttaatactc ttattgggat agctaatcta |
| 1321 | aaaaaggtat tgtctgtttg ggaaagcaac aaactcacta atacctctga gaagttttgg |
| 1381 | caaagtgtct taaaagaaaa cacttggatt ttgagtcaga ttttctctaa cccgactgtt |
| 1441 | ttgataaatg atgaagccta tgtaggcgga aagactgtca aaaatgatag tggtaagtta |
| 1501 | gtagatttct tatatgcaaa ccctttctca aaagatgctg tacttattga gattaagact |
| 1561 | ccatcaacgc ctctaattac tcccactgaa tacagaacag gagtatattc cgcacataaa |
| 1621 | gacttgacag gagcagttac gcaagtgttg acttataaga caaccctaca gagggagtat |
| 1681 | cagaatatag actataacaa ttatagacag ggaatcaaaa ctgattttga cattattact |
| 1741 | ccttgttgtg ttgttatagc aggcatgttt gatacattaa ctgatacagc tcatagacat |
| 1801 | tcctttgaac tgtacagaaa agagttaaaa aatgtaactg taattacttt tgatgaactg |
| 1861 | tttgaaaggg ttaaggggtt aatcaagcta ttggaaggat aaacccttgc cttctttttt |
| 1921 | tactgcctct aaatttatag aatgcatccg ttaaagctaa gtagaaatat tagaagaaag |
| 1981 | ggcaaaattt aaagagagaa atttgccttt tctttttatc aaactgatca aaaaatttta |
| 2041 | tatttcaatc actttatttc gccacctgaa tttttttatt ttttaaaagt gccttgcctc |
| 2101 | tcaccaattc aggtcattat aaaacaaaaa aagaaccgtt tatcaacaac aaacggtccg |
| 2161 | attaaaaatc ttgtgcgtat ctattaaaaa taacttaata aaacatgctt att. |

The coding region for the sduA gene sequence within this embodiment of a Shedu cassette (SEQ ID NO: 9) is as follows: nucleotides 760-1902 encode an embodiment of a SduA polypeptide.

In some embodiments, a Defense System V comprising a SduA polypeptide is encoded by the nucleic acid sequence set forth in SEQ ID NO: 9. In some embodiments, a Defense System V comprising a SduA polypeptide, is encoded by a nucleic acid sequence having at least 80% homology to the sequence set forth in SEQ ID NO: 9. In some embodiments, a Defense System V comprising a SduA polypeptide, is encoded by a nucleic acid sequence having at least 85% homology, or 90% homology, or 95% homology, or 97% homology, or 98% homology, or 99% homology to the sequence set forth in SEQ ID NO: 9. In some embodiments, a Defense System V comprising a SduA polypeptide, is encoded by a nucleic acid sequence having at least 80% identity to the sequence set forth in SEQ ID NO: 9. In some embodiments, a Defense System V comprising a SduA polypeptide, is encoded by a nucleic acid sequence having at least 85% identity, or 90% identity, or 95% identity, or 97% identity, or 98% identity, or 99% identity to the sequence set forth in SEQ ID NO: 9.

In some embodiments, the components of a Shedu system may be identified by the presence of similar domains present within each component. In some embodiments, domains comprise pfam domains and or clusters of orthologous groups (COG) domains.

In some embodiments, the term “SduA” refers to the polynucleotide or expression product e.g., the polypeptide encoded by the sduA gene. In some embodiments, the term “SduA” refers to a SduA polypeptide. In some embodiments, the sduA gene encodes a polypeptide comprising a pfam14082 domain. In some embodiments, the sduA gene encodes a polypeptide comprising a DUF4263 domain. In some embodiments, the sduA gene encodes a polypeptide comprising a pfam01939 domain. In some embodiments, the sduA gene encodes a polypeptide comprising a pfam14082 domain, a DUF4236 domain, or a pfam01939 domain, or any combination thereof. In some embodiments, the SduA polypeptide comprises a pfam14082 domain. In some embodiments, the SduA polypeptide comprises a DUF4263 domain. In some embodiments, the SduA polypeptide comprises a pfam01939 domain. In some embodiments, the SduA polypeptide comprises a pfam14082 domain, a DUF4263 domain, or pfam01939 domain, or any combination thereof.

In some embodiments, a SduA polypeptide is about 367 amino acids long (median gene size).

In some embodiments, the SduA polypeptide comprises the amino acid sequence having at least 80% homology to a sequence selected from the group consisting of the polypeptides referenced in Table 13, rows 2-1247, columns H and I. In some embodiments, the SduA polypeptide comprises the amino acid sequence having at least 80% homology within similar domain regions within a sequence selected from the group consisting of the polypeptides referenced in Table 13, rows 2-1247, columns H and I. In some embodiments, the SduA polypeptide comprises a homolog of a polypeptide having the amino acid sequences set forth in the polypeptides referenced in Table 13, rows 2-1247, columns H and I.

In some embodiments, a SduA polypeptide homolog comprises an endonuclease NucS domain. In some embodiments, a SduA polypeptide homolog comprises an endonuclease NucS activity. In some embodiments, a SduA polypeptide homolog comprises an activity that cleaves both 3′ and 5′ ssDNA extremities of branched DNA structures. In some embodiments, a SduA polypeptide homolog binds to ssDNA. In some embodiments, a SduA polypeptide homolog comprises an activity that cleaves both 3′ and 5′ ssDNA extremities of branched DNA structures and it binds to ssDNA. In some embodiments, an SduA polypeptide comprises a member of the PD-(D/E)XK superfamily.

In some embodiments, the SduA polypeptide comprises the amino acid sequence having at least 80% identity to a sequence selected from the group consisting of the polypeptides referenced in Table 13, rows 2-1247, columns H and I. In some embodiments, the SduA polypeptide comprises the amino acid sequence having at least 80% identity within similar domain regions within a sequence selected from the group consisting of the polypeptides referenced in Table 13, rows 2-1247, columns H and I.

In some embodiments, the SduA polypeptide comprises an amino acid sequence having at least 80%, at least 85%, at least 90%, or at least 95% homology to a sequence selected from the group consisting of the polypeptides referenced in Table 13, rows 2-1247, columns H and I. In some embodiments, the SduA polypeptide comprises the amino acid sequence having at least 80%, 85%, 90%, 95% homology within similar domain regions within a sequence selected from the group consisting of the polypeptides referenced in Table 13, rows 2-1247, columns H and I. In some embodiments, the SduA polypeptide comprises an amino acid sequence having at least 80%, at least 85%, at least 90%, or at least 95% identity to a sequence selected from the group consisting of the polypeptides referenced in Table 13, rows 2-1247, columns H and I. In some embodiments, the SduA polypeptide comprises the amino acid sequence having at least 80%, 85%, 90%, 95% identity within similar domain regions within a sequence selected from the group consisting of the polypeptides referenced in Table 13, rows 2-1247, columns H and I. In some embodiments, the SduA polypeptide comprises an amino acid sequence selected from the group consisting of the polypeptides referenced in Table 13, rows 2-1247, columns H and I.

In some embodiments, the SduA polypeptide comprises an amino acid sequence encoded by a nucleic acid sequence having at least 80%, at least 85%, at least 90%, or at least 95% homology to a sequence selected from the group consisting of the polynucleotides referenced in Table 13, rows 2-1247, columns H and J. In some embodiments, the SduA polypeptide comprises the amino acid sequence encoded by a nucleic acid sequence having at least 80%, 85%, 90%, 95% homology within similar domain regions within a sequence selected from the group consisting of the polynucleotides referenced in Table 13, rows 2-1247, columns H and J. In some embodiments, the SduA polypeptide comprises an amino acid sequence encoded by a nucleic acid sequence having at least 80%, at least 85%, at least 90%, or at least 95% identity to a sequence selected from the group consisting of the polynucleotides referenced in Table 13, rows 2-1247, columns H and J. In some embodiments, the SduA polypeptide comprises the amino acid sequence encoded by a nucleic acid sequence having at least 80%, 85%, 90%, 95% homology within similar domain regions within a sequence selected from the group consisting of the polynucleotides referenced in Table 13, rows 2-1247, columns H and J. In some embodiments, the SduA polypeptide comprises an amino acid sequence encoded from a nucleic acid sequence selected from the group consisting of the polynucleotides referenced in Table 13, rows 2-1247, columns H and J.

In some embodiments, the nucleic acid sequence of a sduA gene comprises a nucleic acid sequence having at least 80%, at least 85%, at least 90%, or at least 95% homology to a sequence selected from the group consisting of the polynucleotides referenced in Table 13, rows 2-1247, columns H and J. In some embodiments, the sduA gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 13, rows 2-1247, columns H and J. In some embodiments, the nucleic acid sequence of a sduA gene comprises a nucleic acid sequence having at least 80%, at least 85%, at least 90%, or at least 95% identity to a sequence selected from the group consisting of the polynucleotides referenced in Table 13, rows 2-1247, columns H and J. In some embodiments, the sduA gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 13, rows 2-1247, columns H and J. In some embodiments, the nucleic acid sequence of a sduA gene comprises a nucleic acid sequence selected from the group consisting of the polynucleotides referenced in Table 13, rows 2-1247, columns H and J.

A skilled artisan would appreciate that the terms a “functional portion of a Defense System V component” or “functional fragment of Defense System V component” or “functional portion of a Shedu defense system component” or “functional fragment of Shedu defense system component” refers to a functional portion of a Shedu polynucleotide or polypeptide, as disclosed herein, which expression is sufficient to elicit an anti-phage activity alone.

The terms “SduA” and “sduA”, also refer to functional SduA and sduA, homologs, which exhibit the desired activity (i.e., conferring phage resistance). Such homologs can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide sequences referenced in Table 13 rows 2-1247 columns H and I, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polynucleotide sequences referenced in Table 13 rows 2-1247 columns H and J, respectively. In some embodiments, such homologs comprise at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% homology or identity within similar domain regions of the polypeptide sequences referenced in Table 13 rows 2-1247 columns H and I, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or homology within nucleotide sequences encoding similar domain regions to the sequences referenced in Table 13 rows 2-1247 columns H and J, respectively.

Table 13 presents embodiments of components of Defense System V that may be found in a diverse array of bacteria and archaea genomes (referenced in Table 18). Table 13 presents embodiments of nucleic acid sequences encoding gene cassettes of Defense System V (referenced in Table 18).

The Gabija Defense System

In some embodiments, a defense system disclosed herein comprises a Gabija anti-phage defense system. (Table 14)

In some embodiments, a Gabija defense system provides a host cell with resistance to entry of foreign nucleic acid invasion. In some embodiments, a host cell expressing a functional Gabija defense system (Defense System VI) provides the host cell resistance foreign nucleic acid invasion.

In some embodiments, a Gabija defense system provides a host cell with resistance to at least one phage.

In some embodiments, a microbial cell comprising a Gabija defense system protects the microbial cell from phage infection. In some embodiments, a microbial cell expressing a Gabija defense system protects the microbial cell from phage infection. In some embodiments, a microbial cell expressing a functional Gabija defense system protects the microbial cell from phage infection. In some embodiments, a bacterial cell comprising a Gabija defense system protects the bacterial cell from phage infection. In some embodiments, a bacterial cell expressing a Gabija defense system protects the bacterial cell from phage infection. In some embodiments, a bacterial cell expressing a functional Gabija defense system protects the bacterial cell from phage infection.

In some embodiments, a Gabija defense system (Defense System VI) provides a host cell with resistance to plasmid transformation In some embodiments, a host cell expressing a functional Gabija defense system (Defense System VI) provides a host cells with resistance to plasmid transformation.

In some embodiments, a Gabija defense system (Defense system VI) provides a host cell with resistance to entry of conjugative elements. In some embodiments, a host cell expressing a function Gabija defense system (Defense System VI) provides the host cell resistance from entry of conjugative elements.

As used herein, the term “a Gabija anti-phage defense system” may be used interchangeably with the term “a Defense System VI”, having all the same meanings and qualities. A skilled artisan would appreciate that the term “Gabija system” may be used interchangeably in some embodiments with “Gabija defense system”, “Gabija defense system”, “Gabija anti-phage system”, and “Defense System VI”, having all the same meanings and qualities.

In some embodiments, a microbial species does not comprise an endogenous Defense System VI. In some embodiments, a microbial species does not express an endogenous Defense System VI. In some embodiments, a microbial species does not express an endogenous functional Defense System VI.

In some embodiments, a bacterial species does not comprise an endogenous Defense System VI. In some embodiments, a bacterial species does not express an endogenous Defense System VI. In some embodiments, a bacterial species does not express an endogenous functional Defense System VI.

A Gabija defense system comprises, in some embodiments, a nucleic acid construct comprising a nucleic acid sequence encoding a GajA polypeptide comprising a pfam13175 domain or a COG3593 domain or a combination thereof, and a GajB polypeptide comprising a pfam04257 domain or a pfam00580 domain or a pfam13361 domain or a combination thereof. In some embodiments, a Gabija defense system comprises a nucleic acid construct comprising a nucleic acid sequence comprising a gajA gene and a gajB gene.

In some embodiments, a Gabija defense system comprises, a GajA polypeptide comprising a pfam13175 domain or a COG3593 domain or a combination thereof, and a GajB polypeptide comprising a pfam04257 domain or a pfam00580 domain or a pfam13361 domain or any combination thereof. In some embodiments, a Gabija defense system comprises a GajA polypeptide and a GajB polypeptide.

In some embodiments, a Defense System VI comprises a GajA polypeptide comprising a pfam13175 domain or a COG3593 domain or a combination thereof, or comprising an amino acid sequence having at least 80% identity to a sequence selected from the group referenced in Table 14 rows 2-4599 columns H and I; or a GajB polypeptide comprising a pfam04257 domain or a pfam00580 domain or a pfam13361 domain or any combination thereof or comprising an amino acid sequence having at least 80% identity to a sequence selected from the group referenced in Table 14 rows 2-4599 columns L and M; or a combination thereof.

In some embodiments, a Defense System VI comprises at least two different polypeptide components selected from a GajA polypeptide comprising a pfam13175 domain or a COG3593 domain or a combination thereof, or comprising an amino acid sequence having at least 80% identity to a sequence selected from the group referenced in Table 14 rows 2-4599 columns H and I; and a GajB polypeptide comprising a pfam04257 domain or a pfam00580 domain or a pfam13361 domain or any combination thereof or comprising an amino acid sequence having at least 80% identity to a sequence selected from the group referenced in Table 14 rows 2-4599 columns L and M.

In some embodiments, a Gabija defense system having an anti-phage activity comprises a nucleic acid construct comprising a nucleic acid sequence encoding a GajA polypeptide and a GajB polypeptide. In some embodiments, a Gabija defense system having an anti-phage activity comprises a nucleic acid construct comprising a nucleic acid sequence encoding a GajA polypeptide comprising an ATPase domain, and a GajB polypeptide comprising a helicase activity. In some embodiments, a Gabija defense system having an anti-phage activity comprises a nucleic acid construct comprising a nucleic acid sequence encoding a GajA polypeptide comprising an ATPase domain. In some embodiments, a Gabija defense system having an anti-phage activity comprises a nucleic acid construct comprising a nucleic acid sequence encoding a GajB polypeptide comprising a helicase activity. In some embodiments, a Gabija defense system having an anti-phage activity comprises a nucleic acid construct comprising a nucleic acid sequence encoding a GajA polypeptide comprising an ATP-dependent endonuclease, and a GajB polypeptide comprising a UvrD helicase activity. In some embodiments, a Gabija defense system having an anti-phage activity comprises a nucleic acid construct comprising a nucleic acid sequence encoding a GajA polypeptide comprising an ATP-dependent endonuclease activity. In some embodiments, a Gabija defense system having an anti-phage activity comprises a nucleic acid construct comprising a nucleic acid sequence encoding a GajB polypeptide comprising a UvrD helicase activity.

In some embodiments, the Gabija anti-phage defense system comprises a nucleic acid construct comprising nucleic acid sequences encoding a cassette comprising gajA and gajB genes.

In some embodiments, a Gabija (Defense System VI) gene cassette comprises the nucleic acid sequence:

(SEQ ID NO: 5; construct 7 of Table 4)

| | |
|---|---|
| 1 | gatcaaacaa tttattccta ttggaatggg ggaagagtcc caaactctca aagagctagt |
| 61 | tgatattgtt tggctgctgt gatggattta tttctaagaa aatagtgtgg gctattcatt |
| 121 | tttatgttgg atgtttgcag acttaataat gatagcttta gggaacactt actctgtaaa |
| 181 | atcggttatt ccctaaaccg ggatttaggt tcacagtata caagctctgg ggttacagaa |
| 241 | tacacttaat cttacagcat cattcaacca aaagagtctc tttatgataa tgcctgtgtc |
| 301 | gagtcatttt acggtattaa aaaaagaaga agtaaattat gtgcaatatc tagactacca |
| 361 | attttccaag ataagaaata tatgttttac gtttcggaaa tattagttgt atcaatggtt |
| 421 | tatagagcat tctacatttt cttgtgcaaa tcatgtaaca cagaaaaatg aagtttatga |
| 481 | aaaatattta tttatttttc actggtgaat actgaaatag tattcattta ttttttgttt |
| 541 | attttacaaa tttttttgaa agtcgttcaa tagcagagaa tttctagtat aattttggta |
| 601 | aataattcta tagattgatc ggaattatga atttaattta cagatatttt agagtaacat |
| 661 | tggagctatt atattaaaaa agaatgcttc aagattgagc gaacgatagc tttgggataa |
| 721 | attagctttt tggcggatag tgaagcagta acgagtaagt tagtaaataa agtgttaaat |
| 781 | agaataacag aagaatagga gagggcttta tgtatttaaa aagtttaaaa atctacaact |
| 841 | ttagaaaatt tggaacaaat aacaacaaga tagaatttgt tgatgctaaa agttttcaag |
| 901 | aacaaagaaa taaaaaagaa gtaaatattg ctccaacaac aactttaatt gtaggaaaga |
| 961 | ataactgtgg aaaaacaacg attattaaat cgttagataa tcttattaat aataataaag |
| 1021 | acgcttttaa agcaaatgat tttaattttt tatacttaaa aaaactaatt gagtattatg |
| 1081 | aacaagcaat tccaaaatta gatgatgaat taaaagaaaa aattaaaaca ccgtatcttc |
| 1141 | aatttaatat ttgtattgga attgagaata atagtaatga cttagttacc aatatagttc |
| 1201 | gttttatgaa attagatgat attaataatt ctgaattaaa aattgttata aaagtagaat |
| 1261 | tggagaacga agaagttttc attagagatg tgaaaaaact gttagagaaa aatgataatt |
| 1321 | cgagtcttcg gtttaatagc tttttggaat tgataaatga ttcggatttt aaaataaatt |
| 1381 | attataatac agataatgat aaaatagata attttaacat aaaaaattta attgaattga |
| 1441 | gaccgattaa agcaaataac attgaaagcg aaaaatgcct ttcaaaagct tttagtaaaa |
| 1501 | taattgaata tcgctataaa acattattcg aagaggaaag cgataatctt aattcgacaa |
| 1561 | tcataaatat taatcgaaca cttaccaaat taattagtga aaaacatacg gttagtataa |
| 1621 | ataagtcttt agaagaaatt gaatcaaatg aaaaacttca agtattgcta agggcagatt |
| 1681 | tgacttttca aaatgtgatg aataatttaa tcaaatatga atacgtcgaa cgtaatatga |
| 1741 | atatacctga gaatcaattt ggtttaggtt atacaaattt gatgatgata atagcggatt |
| 1801 | taattgatta tatggaaaaa tacccagaaa actcatttaa cagtaaggtt aatttaattt |
| 1861 | caattgaaga acctgaaaca tttatgcatc cacaaatgca ggagttattt ataaaaaata |
| 1921 | taaatgaagc gattgcttct ttattgaaaa gtaaaaataa aaacgtaaat agccagttga |
| 1981 | taattacaac acactcatca catatactaa atagtaaaat acacagtggt aatactttta |
| 2041 | ataatataaa ttacgttact accaaaaata attatactca tgtagtgaat ttgcatgatg |
| 2101 | atataattat ccctaaaagt gaaaaaaaag ctcataaaag agagcaggat cttaagttct |
| 2161 | taaaaaaaca tattaagtat aaagtttctg agttgttttt ttcagatgct attatttttg |
| 2221 | ttgaaggtgt gactgaagaa acgcttctta aatattatat tgatgataat aataagctca |
| 2281 | ataaatatta tatatcggtt ttcaatattg atggggctca cggaatggtt tatcacaatt |
| 2341 | taattgaaat actacaagta cctgctttga taattacgga tttagatata gaaagagatg |
| 2401 | atgatgaaaa gaaaaatttc aagcaaatct ctgatttaac gggaaaattc acaactaata |
| 2461 | aaacaattaa aaagtataat gatgactcta atagtttaga ggaattacag catgagcagc |
| 2521 | ttaaaataaa taatatgtat attgcatatc aaggtaaaat cgaagaatat tatgcaacaa |
| 2581 | gttttgaaga agcatttatc ttaacaaatt acaaaaatga acttttaaat agtgtactaa |
| 2641 | aaaaaatgaa acctcagatt tatgaaaata ttatgggtga gggagatgat ttcacaaaga |
| 2701 | taaaggaaaa ttcctataaa ctccaaaaaa agttatcaaa tgataaaagt gattttgcga |
| 2761 | atgagctact ctataagttt ataacggagg atataacaat tccttctttg ccaaagtata |
| 2821 | ttaagagtgg cctttcatgg ttagctaaaa aattagaagg ggaggaataa gggtgagttt |
| 2881 | atccaaaata atcccatcta ataagaaaga agagcagaaa tcaataaatg caatttttaa |
| 2941 | ttctattgat aaaagtgaag gtatcatttt taattctggt gctggtgctg gaaaaacata |
| 3001 | cgctcttatt gaaagtttga aatacataat aagaaactac gagaagagct taaaacaaca |
| 3061 | taatcaacaa attatctgta tcacatatac gaatgttgca actaaagaag taaaagagag |
| 3121 | attaggaaac acagatttag tattagtttc tacaattcat gaaagaatgt ggggactcat |
| 3181 | taaagattat caaaaagaat tagttgagat tcacaaagaa aagttggaag atgaaatctc |
| 3241 | aagtttaaaa caaaaattag aaaaaggtca gggatacgaa aaatttcaag aattagaaga |
| 3301 | ggacttaaag ggaaatttta aaaaaataat gattgagaat agggaactat tttttcaaaa |
| 3361 | ttatagtgct aaagcagcag aggtaaaaaa atcatttaaa atactcttaa acgattaccc |
| 3421 | taatatgctg aaaaatgttg gaaattttaa gagaatagta aatgcaattt acaaattaga |
| 3481 | tcagtatagt aaatgctatg aaaatataag cttaaataaa cagggttata aatctgtaga |
| 3541 | gtacaattcg atttataata acgatcaact ttataaaatg agaataagcc atgatacttt |
| 3601 | gttagattat ggtctgaaaa taattaagaa atatgacttg ttaaaacaaa taatcattga |
| 3661 | taaatatcca tttattttta ttgatgagta tcaggatacg gatgaaaaag ttatattaat |
| 3721 | aatgagttat ttagagcaat atgcaaaaaa aattgatcac aaaattttta taggttattt |
| 3781 | tggtgatact gctcaaaata tttatgatga tggagtagga agtgaaataa caaagattca |
| 3841 | ctcaggatta aaacagatag ataaagtgtt taatagaaga tctacaaaag aagtcattga |
| 3901 | ggttataaac aaaattagaa acgatagcat cgaacaaata tcaatttatg atgattgtga |
| 3961 | aggcggtagt gtaaagtttt ataaagggac ttcaggtaat gttaaggact taatagagcg |
| 4021 | gtatatatat gaatggaaaa taactacgga aaatcaacta cattgtctag ttttaactaa |
| 4081 | taagattgtt gcagaatata gtggttttaa aaacatatac gaagctttca aagagacaga |
| 4141 | taaatataaa gggagcaatt ataatcaact taatacggaa ctcttaagta atgatttatc |
| 4201 | taagcttggt gaaattccta agttgttatt taatatagtt agattacaga ataatcttgt |
| 4261 | ggataaaacg acttcagtta ttgatattac acctaaagaa agcctattcg atgagatgag |
| 4321 | tatagaaggg ttaaggaatt taattaaact attaaaacaa taccaaggga aaacattagg |
| 4381 | tgaatatata gaatcgatat caactgttta ttctcaagtt aatgatgaaa attataaaaa |
| 4441 | aataatagat tggacttttg gttttgaaaa tatcacattc gaactattta aaaatcatct |
| 4501 | tatagaaaag ttatttaata atatattaga tgatgatgtt gatcgtgcaa ccgcaactat |
| 4561 | acaaaaaata ctagaagttg atattgttga gtatggattg tggtataaat ttattatgga |
| 4621 | taaacaagaa gagaaggtta tttatcatac ttatcatggt acaaagggac gagagtttga |
| 4681 | taatgttatt attattatgg aaaatgcttt tggaagaaat cacaattatt ttaatttctt |
| 4741 | ttttgaaaat ttcttgcatc cagatgtatt agaaggtgaa aagaaactga acttcgagaa |
| 4801 | aataaagaat ttgttatatg tttcttgctc tagagctatt aagaatttaa gagttttata |
| 4861 | tattgatgat gttaccgatt ttgaaagtga aattaaggaa atgtttggag aagtatatcc |
| 4921 | tttttagtaa taattgaatg aagatatttt tcatgaataa aaaaatgttg tgttgatggg |
| 4981 | aagcatcata aaaaaacact cttaaccttt ttacaaagaa acaccccact cgttaggctt |
| 5041 | aattattaag tttatgtatt attttattat tcaaaacctt atttttgcta ttgattagcc |
| 5101 | gctcataata tttaattatg agcggtttta taacatccga gcacaagttt taaaaatgaa |
| 5161 | aattcaggac taggtttatt attcaagacg atgattacat atcatttaag gtgcaggaaa |
| 5221 | ataattaaag cattataagt aatatcgata tttcagtttg ttaagatttt ctaagggaaa |
| 5281 | tttgttcttt tatctagaaa ataattggat tatctaattc atcat. |

The coding regions for each of the gajA and gajB gene sequences within this embodiment of a Gabija cassette (SEQ ID NO: 5) are as follows: nucleotides 810-2870 encode an embodiment of a GajA polypeptide, and nucleotides 2873-4927 encode an embodiment of a GajB polypeptide.

In some embodiments, a Defense System VI comprising a GajA polypeptide and a GajB polypeptide is encoded by the nucleic acid sequence set forth in SEQ ID NO: 5. In some embodiments, a Defense System VI comprising a GajA polypeptide and a GajB polypeptide, is encoded by a nucleic acid sequence having at least 80% homology to the sequence set forth in SEQ ID NO: 5. In some embodiments, a Defense System VI comprising a GajA polypeptide and a GajB polypeptide, is encoded by a nucleic acid sequence having at least 85% homology, or 90% homology, or 95% homology, or 97% homology, or 98% homology, or 99% homology to the sequence set forth in SEQ ID NO: 5. In some embodiments, a Defense System VI comprising a GajA polypeptide and a GajB polypeptide, is encoded by a nucleic acid sequence having at least 80% identity to the sequence set forth in SEQ ID NO: 5. In some embodiments, a Defense System VI comprising a GajA polypeptide and a GajB polypeptide, is encoded by a nucleic acid sequence having at least 85% identity, or 90% identity, or 95% identity, or 97% identity, or 98% identity, or 99% identity to the sequence set forth in SEQ ID NO: 5.

In some embodiments, a Gabija (Defense System VI) gene cassette comprises the nucleic acid sequence:

(SEQ ID NO: 6; construct 8 of Table 4)

| | |
|---|---|
| 1 | ataatagtac tatttaacga tacctgtcaa aaaacataat cgatatttta tcaaaattaa |
| 61 | aaaaagaaac cattttattt aacttttctt taaaatggtt tcttggatta tgggacttag |
| 121 | ttttttgtct ggaaaatata atgggaattt attgtataat aaaactatgt agaaatacat |
| 181 | ggttttttta tttattgagg atgggattct agtgaagggg aacagcttgc ttaactattt |
| 241 | ataaaataca gggataagaa cagaattaaa gaaggtatat gttaaaagta aaataattaa |
| 301 | gaagataagg tgatgaataa tgaaattcag taatattaca ataaagaact tcaggaattt |
| 361 | tgaaaaagta aatataaatt tagataataa aaatgtgatt ttcgggatga atgatattgg |
| 421 | aaaaacaaat tttttatatg cattgagatt tcttttagat aaagagataa gaaaattcgg |
| 481 | ttttaataaa tctgattatc ataaacatga cacttctaaa aaaattgaaa ttattttaac |
| 541 | acttgatttg tctaattatg aaaaggatga agatacaaaa aaacttattt cagtggttaa |
| 601 | gggtgctaga acatcggcaa atgcagatgt tttttatatc gcactagaat ctaaatatga |
| 661 | tgataaagaa ttatatggga acataatttt aaaatgggga tcggaactag ataatttaat |
| 721 | agatatacca gggagaggga acataaacgc gttagataat gtatttaagg tgatttatat |
| 781 | aaatccgctt gttgatttag acaaattgtt cgcacaaaat aaaaaatata tttttgaaga |
| 841 | gtcacagggt aatgaatcag atgaagggat tttaaataat attaaatctt taacagatca |
| 901 | agtaaatcaa caaataggag aaatgacaat tattaagggt ttccagcaag agataacaag |
| 961 | tgaatatagg tctttaaaaa aagaagaggt ttctattgag ctgaagtccg aaatggcaat |
| 1021 | taaaggattt ttctcagata ttattccata tataaaaaaa gacggtgatt ctaattacta |
| 1081 | tccaacctca ggggatggta gaagaaaaat gctttcttac tctatatata actatctggc |
| 1141 | taagaaaaaa tatgaggata aaattgttat ttatttaatt gaggaacccg aaattagtct |
| 1201 | acatagatca atgcaaattg ctttatcaaa acagttattt gaacaatcta catataaata |
| 1261 | ttttttctta tccactcact ctcctgaact tctttatgaa atggataata caagattaat |
| 1321 | aagagtgcat tcaactgaaa aggttgtatg ttcttcccat atgtataatg tggaagaagc |
| 1381 | ctatggaagt gtcaagaaaa agctaaataa agctttatca tcggctctat ttgctgaaag |
| 1441 | agtactttta atagaaggtc cttcagaaaa aatattattt gaaaaggttt tagacgaagt |
| 1501 | agaaccagaa tatgaattaa atggaggttt cttgcttgaa gtaggaggga cgtactttaa |
| 1561 | tcattatgtg tgtacattaa atgatttagg tataacccat ataattaaaa cagataatga |
| 1621 | tttgaaatca aaaaaaggta aaaaaggtgt atacgaatta ctaggattaa atagatgctt |
| 1681 | aaacttatta ggacgtgaaa atctagatga gattactatt gacatccctg aagatataaa |
| 1741 | aggtaagaag aaaaaagaga gacttaatga aagaaaaaaa gagattttta aacaatataa |
| 1801 | aaatgaggta ggggaattct taggggaacg aatatattta tcggaaatcg atctggaaaa |
| 1861 | tgatttatat tctgcaattg gtgaaagcat gaaaagaatt tttgaaaacg aagatcccgt |
| 1921 | gcactattta cagaaaagta aactatttaa catggtcgag ctagtaaata atttaagtac |
| 1981 | taaagattgt tttgatgttt ttgagcacga aaaatttgca tgcctaaagg agttggtggg |
| 2041 | tagtgataga ggatgaaatg tctagagaac aaataataaa ggatgggggt aatattcttg |
| 2101 | ttaccgctgg agcaggttcg ggtaaaacaa caatattagt tagtaaaatt gaagctgatt |
| 2161 | taaaagaaaa taaaactcat tactcaattg cagctgttac ttttacaaat aaggcagcaa |
| 2221 | aagaaatcga gggaagatta gggtattcat caagagggaa ttttattggc actaacgatg |
| 2281 | gttttgtcga gtctgaaatt attaggccgt ttattaaaga tgcatttgga aatgattatc |
| 2341 | cagacaattt cactgctgaa tattttgata accaatttgc ttcatacgat aagggattgc |
| 2401 | aagtgctaaa atatcaaaat atattaggga cttatagtaa tcctaaaaag aattttaagt |
| 2461 | ttcaattggc tttagatatt ttaaaaaaat cacttgtcgc tagacaatat atattttcaa |
| 2521 | aatacttcaa gatatttata gacgagtacc aagattcgga taaggatatg cataatttat |
| 2581 | ttatgtattt aaaggatcag cttaaaatta agttatttat tgttggtgac ccaaaacaat |
| 2641 | ctatttatat ctggagggga gcagaacctg aaaattttaa tggtcttata gaaaattcta |
| 2701 | cggattttaa taaatatcat ttaacttcca actttcgatg ctgtcaggat attcaaaatt |
| 2761 | actctaattt atttaatgaa gaaactagaa gcttaattaa agaaaaaaat gaggttcaaa |
| 2821 | atgtaatcag tatagcagac gatatgccaa tttcagatat tttattaaaa ttaacagaag |
| 2881 | aaaagcaggt attaaacata gaagcggaat tagtgatttt agtccggaga cgtaatcaag |
| 2941 | ccattgaaat aatgaaagaa ctaaatgaag aagggtttaa ttttattttt attccccaaa |
| 3001 | ccccattaga tagggcaact ccaaatgcaa ctcttttaaa agaggtaatt aaatatgtta |
| 3061 | aaaatgatag atattcaata tatgatttag ctgctgaaat tgtaggtaat ctaagttcac |
| 3121 | gagaaattaa ggagatacaa aaaataatta atgaattact agtacctaat attaatcagg |
| 3181 | tactaattaa tcaggtatta attaatttat ttgctaaatt agaaattact ttagatacta |
| 3241 | gagaaattac agcatttaca gaagtaatga tgacgaatga atttgacata gcatttgata |
| 3301 | caaatgaata tttacataaa atatttactg tacattctgc aaaaggatta gaatttaatc |
| 3361 | aagtcattat tactgcaagt gattacaatg tacactataa tagagatact aacgaacatt |
| 3421 | atgttgctac aactagagca aaagataaat taattgtcat tatggataat aagaagtact |
| 3481 | cagattatat tgagacgcta atgaaagaac ttaaaattaa aaatattatt aagtcaatat |
| 3541 | aaggaaactt aagtttgaca taataacgat gaaatataaa agtttgtttt cgtaaatagc |
| 3601 | caatgtcact aagttatcaa gacttaaatt ttaaaacctt taaaaataaa gactctaaca |
| 3661 | tattagtcat aattaatatg ttagggtctt ttgttttatt tgaaatgtta ttttcaactt |
| 3721 | tataagacaa tgaatattat ttttgtttaa taatatccag agtgctcaaa ataaatatca |
| 3781 | gaaggtaatt cctaataata ttgcgtgtgt agttaatagt taaaataata aaataaaaaa |
| 3841 | agatggtata atttagtata ccatcttttt ttattttatt aggaggttca aaa. |

The coding regions for each of the gajA and gajB gene sequences within this embodiment of a Gabija cassette (SEQ ID NO: 6) are as follows: nucleotides 320-2056 encode an embodiment of a GajA polypeptide, and nucleotides 2058-3542 encode an embodiment of a GajB polypeptide.

In some embodiments, a Defense System VI comprising a GajA polypeptide and a GajB polypeptide is encoded by the nucleic acid sequence set forth in SEQ ID NO: 6. In some embodiments, a Defense System VI comprising a GajA polypeptide and a GajB polypeptide, is encoded by a nucleic acid sequence having at least 80% homology to the sequence set forth in SEQ ID NO: 6. In some embodiments, a Defense System VI comprising a GajA polypeptide and a GajB polypeptide, is encoded by a nucleic acid sequence having at least 85% homology, or 90% homology, or 95% homology, or 97% homology, or 98% homology, or 99% homology to the sequence set forth in SEQ ID NO: 6. In some embodiments, a Defense System VI comprising a GajA polypeptide and a GajB polypeptide, is encoded by a nucleic acid sequence having at least 80% identity to the sequence set forth in SEQ ID NO: 6. In some embodiments, a Defense System VI comprising a GajA polypeptide and a GajB polypeptide, is encoded by a nucleic acid sequence having at least 85% identity, or 90% identity, or 95% identity, or 97% identity, or 98% identity, or 99% identity to the sequence set forth in SEQ ID NO: 6.

In some embodiments, a construct comprising the Gabija defense system encodes one component of the defense system, whereby multiple constructs, for example but not limited to, two constructs may be used to assemble the functional defense system. In some embodiments, the components of a Gabija defense system comprise genes gajA and gajB. In some embodiments, the components of a Gabija defense system consist of genes gajA and gajB. In some embodiments, the components of a Gabija defense system comprise nucleic acid sequences encoding a GajA polypeptide and a GajB polypeptide. In some embodiments, the components of a Gabija defense system consist of nucleic acid sequences encoding a GajA polypeptide and a GajB polypeptide.

In some embodiments, a construct comprising the Gabija defense system encodes one component of the defense system, whereby a second construct may be used to assemble the functional defense system. In some embodiments, a construct comprising the Gabija defense system encodes one component of the defense system, whereby additional constructs may be used to assemble the functional defense system. For example, in some embodiments each of GajA and GajB may be encoded by nucleic acid sequences comprised in different constructs, wherein expression of the combination of constructs produces a functional Gabija defense system.

In some embodiments, the components making up a functional Gabija anti-phage defense system comprise a GajA polypeptide and a GajB polypeptide, each encoded by a gajA and a gajB gene, respectively. In some embodiments, a gajA gene encodes a polypeptide comprising an ATPase activity. In some embodiments, a gajB gene encodes a polypeptide comprising a helicase activity.

In some embodiments, a Gabija defense system having an anti-phage activity comprises a nucleic acid encoding a GajA polypeptide. In some embodiments, a Gabija defense system having an anti-phage activity comprises a nucleic acid encoding a GajA polypeptide and a GajB polypeptide. In some embodiments, a Gabija defense system having an anti-phage activity comprises a nucleic acid encoding a GajB polypeptide.

In some embodiments, a Gabija defense system having an anti-phage activity comprises a nucleic acid comprising a gajA gene. In some embodiments, a Gabija defense system having an anti-phage activity comprises a nucleic acid comprising a gajA gene and a gajB gene. In some embodiments, a Gabija defense system having an anti-phage activity comprises a nucleic acid comprising gajB gene.

In some embodiments, a Gabija defense system having an anti-phage activity comprises a nucleic acid construct comprising nucleic acids encoding polypeptides in a set order. In one embodiment, the 5′ to 3′ order of polypeptides encoded is GajA and GajB. In some embodiments the 5′ to 3′ order of polypeptides does not affect the anti-phage activity. In some embodiments the 5′ to 3′ order of polypeptides does affect the anti-phage activity.

In some embodiments, the 5′ to 3′ order of polypeptides is random, for example any order of nucleic acid sequences encoding GajA and GajB.

In some embodiments, a Gabija defense system having an anti-phage activity comprises a nucleic acid construct comprising genes in a set order. In some embodiment, the 5′ to 3′ order of genes is gajA and gajB. In some embodiment, the 5′ to 3′ order of genes in a Gabija defense system is not gajA and gajB. In some embodiments the 5′ to 3′ order of genes does not affect the anti-phage activity. In some embodiments the 5′ to 3′ order of genes does affect the anti-phage activity.

In some embodiments, the 5′ to 3′ order of genes is random, for example any order of gajA and gajB.

In some embodiments, the Gabija system (Defense System VI) composition and order is as shown in FIG. 3B.

In some embodiments, a Gabija defense system having an anti-phage activity originates from a microbial genome, for example a bacterial or an archaeal genome (Table 14). A skilled artisan would appreciate that the Gabija system is not present in the majority of bacteria and or archaea species.

In some embodiments, a functional Gabija defense system comprises a construct comprising nucleic acid sequences encoding polypeptides, wherein the nucleic acid sequence encoding each polypeptide may originate from a different microbial species. For example, the source of the nucleic acid sequence encoding a GajA polypeptide may be one microbial species, while the source of the nucleic acid sequence encoding a GajB polypeptide may be a different microbial species. In some embodiments, a functional Gabija defense system comprises a non-naturally occurring combination of polypeptide components. In some embodiments, a functional Gabija defense system comprises a combination of at least two polypeptides that do not naturally occur together.

In some embodiments, a functional Gabija defense system comprises a construct comprising nucleic acid sequences encoding polypeptides, wherein the nucleic acid sequence encoding each polypeptide may originate from a different bacterial species. For example, the source of the nucleic acid sequence encoding a GajA polypeptide may be one bacterial species, while the source of the nucleic acid sequence encoding a GajB polypeptide may be a different bacterial species.

In some embodiments, the source of the nucleic acid encoding a GajA polypeptide and a GajB polypeptide is the same. In some embodiments, the source of the nucleic acid encoding a GajA polypeptide and a GajB polypeptide is the not the same. In some embodiments, the source of the nucleic acid sequence of any of the components of a Gabija defense system comprises any of the species listed in Table 14.

In some embodiments, a Gabija defense system having an anti-phage activity comprises a nucleic acid construct comprising non-coding regions between the nucleic acid sequences encoding the polypeptides. In some embodiments, the non-coding regions between the nucleic acid sequences comprises nucleic acid sequence not naturally occurring in the microbial species of origin. In some embodiments, the non-coding regions between the nucleic acid sequences comprises nucleic acid sequence is the naturally occurring in the microbial species of origin.

In some embodiments, a Gabija system disclosed herein comprises a multi-gene phage resistance system broadly distributed in microbial genomes, for example but not limited to bacteria and archaea. According to some embodiments, the Gabija system components are located in a gene cluster (a cassette of genes) in a microbial cell genome. According to some embodiments, the Gabija system components are located in close proximity (e.g. positioned within 20 genes, 10 genes, 5 genes or less one from the other) in a genome of a prokaryotic cell. According to some embodiments the prokaryotic cell expresses an endogenous Gabija defense system. According to some embodiments, the prokaryotic cell expresses an endogenous functional Gabija defense system. According to some embodiments, the species of prokaryotic cell is selected from the group consisting of the species listed in Table 14. According to some embodiments a prokaryotic cell expresses a non-endogenous Gabija defense system. According to some embodiments, a prokaryotic cell expresses a non-endogenous functional Gabija defense system. According to some embodiments, the species of prokaryotic cell expressing a non-endogenous functional Gabija defense system is selected from the group consisting of the species listed in Table 14.

In some embodiments, Gabija defense system components comprise GajA and GajB polypeptides. In some embodiments, Gabija defense system components comprise functional portions of GajA and GajB polypeptides. In some embodiments, the Gabija defense system functional components are encoded by gajA and gajB genes.

Non-limiting embodiments of endogenous Gabija systems and the respective location of their components are provided in Table 14 herein.

In some embodiments, the components of a Gabija system may be identified by the presence of similar domains present within each component. In some embodiments, domains comprise pfam domains and or clusters of orthologous groups (COG) domains.

In some embodiments, the term “GajA” refers to the polynucleotide or expression product e.g., the polypeptide encoded by the gajA gene. In some embodiments, the term “GajA” refers to a GajA polypeptide. In some embodiments, the gajA gene encodes a polypeptide comprising a pfam13175 domain. In some embodiments, the gajA gene encodes a polypeptide comprising a COG3593 domain. In some embodiments, the gajA gene encodes a polypeptide comprising a pfam13175 domain and a COG3593 domain. In some embodiments, the GajA polypeptide comprises a pfam13175 domain. In some embodiments, the GajA polypeptide comprises a COG3593 domain. In some embodiments, the GajA polypeptide comprises a pfam13175 domain and a COG3593 domain.

In some embodiments, GajA polypeptide is encoded by a gene positioned within 5-60 genes of a gene encoding a GajB polypeptide in a genome of a prokaryotic cell. In some embodiments, GajA polypeptide is encoded by a gene positioned within 5 genes upstream (5′) or downstream (3′) to a gene encoding a GajB polypeptide in a genome of a prokaryotic cell. In some embodiments, GajA polypeptide is encoded by a gene positioned within 5 genes upstream (5′) and downstream (3′) to a gene encoding a GajB polypeptide in a genome of a prokaryotic cell.

In some embodiments, GajA and GajB are encoded by genes positioned sequentially 5′ to 3′ in a genome of a prokaryotic cell. In some embodiments, GajA and GajB are encoded by genes positioned contiguously 5′ to 3′ in a genome of a prokaryotic cell.

In some embodiments, gajA and gajB genes are positioned sequentially 5′ to 3′ in a genome of a prokaryotic cell. In some embodiments, gajA and gajB, genes are positioned contiguously 5′ to 3′ in a genome of a prokaryotic cell.

In some embodiments, a GajA polypeptide is about 670 amino acids long (median gene size).

In some embodiments, the GajA polypeptide comprises the amino acid sequence having at least 80% homology to a sequence selected from the group consisting of the polypeptides referenced in Table 14, rows 2-4599, columns H and I. In some embodiments, the GajA polypeptide comprises the amino acid sequence having at least 80% homology within similar domain regions within a sequence selected from the group consisting of the polypeptides referenced in Table 14, rows 2-4599, column H. In some embodiments, the GajA polypeptide comprises a homolog of a polypeptide having the amino acid sequences set forth in the polypeptides referenced in Table 14, rows 2-4599, columns H and I.

In some embodiments, a GajA polypeptide homolog comprises an ATPase domain. In some embodiments, a GajA polypeptide homolog comprises an ATPase activity. In some embodiments, a GajA polypeptide homolog comprises an ATP-dependent endonuclease domain. In some embodiments, a GajA polypeptide homolog comprises an ATP-dependent endonuclease activity.

In some embodiments, the GajA polypeptide comprises the amino acid sequence having at least 80% identity to a sequence selected from the group consisting of the polypeptides referenced in Table 14, rows 2-4599, columns H and I. In some embodiments, the GajA polypeptide comprises the amino acid sequence having at least 80% identity within similar domain regions within a sequence selected from the group consisting of the polypeptides referenced in Table 14, rows 2-4599, columns H and I.

In some embodiments, the GajA polypeptide comprises an amino acid sequence having at least 80%, at least 85%, at least 90%, or at least 95% homology to a sequence selected from the group consisting of the polypeptides referenced in Table 14, rows 2-4599, columns H and I. In some embodiments, the GajA polypeptide comprises the amino acid sequence having at least 80%, 85%, 90%, 95% homology within similar domain regions within a sequence selected from the group consisting of the polypeptides referenced in Table 14, rows 2-4599, columns H and I. In some embodiments, the GajA polypeptide comprises an amino acid sequence having at least 80%, at least 85%, at least 90%, or at least 95% identity to a sequence selected from the group consisting of the polypeptides referenced in Table 14, rows 2-4599, columns H and I. In some embodiments, the GajA polypeptide comprises the amino acid sequence having at least 80%, 85%, 90%, 95% identity within similar domain regions within a sequence selected from the group consisting of the polypeptides referenced in Table 14, rows 2-4599, columns H and I. In some embodiments, the GajA polypeptide comprises an amino acid sequence selected from the group consisting of the polypeptides referenced in Table 14, rows 2-4599, columns H and I.

In some embodiments, the GajA polypeptide comprises an amino acid sequence encoded by a nucleic acid sequence having at least 80%, at least 85%, at least 90%, or at least 95% homology to a sequence selected from the group consisting of the polynucleotides referenced in Table 14, rows 2-4599, columns H and J. In some embodiments, the GajA polypeptide comprises the amino acid sequence encoded by a nucleic acid sequence having at least 80%, 85%, 90%, 95% homology within similar domain regions within a sequence selected from the group consisting of the polynucleotides referenced in Table 14, rows 2-4599, columns H and J. In some embodiments, the GajA polypeptide comprises an amino acid sequence encoded by a nucleic acid sequence having at least 80%, at least 85%, at least 90%, or at least 95% identity to a sequence selected from the group consisting of the polynucleotides referenced in Table 14, rows 2-4599, columns H and J. In some embodiments, the GajA polypeptide comprises the amino acid sequence encoded by a nucleic acid sequence having at least 80%, 85%, 90%, 95% homology within similar domain regions within a sequence selected from the group consisting of the polynucleotides referenced in Table 14, rows 2-4599, columns H and J. In some embodiments, the GajA polypeptide comprises an amino acid sequence encoded from a nucleic acid sequence selected from the group consisting of the polynucleotides referenced in Table 14, rows 2-4599, columns H and J.

In some embodiments, the nucleic acid sequence of a gajA gene comprises a nucleic acid sequence having at least 80%, at least 85%, at least 90%, or at least 95% homology to a sequence selected from the group consisting of the polynucleotides referenced in Table 14, rows 2-4599, columns H and J. In some embodiments, the gajA gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 14, rows 2-4599, columns H and J. In some embodiments, the nucleic acid sequence of a gajA gene comprises a nucleic acid sequence having at least 80%, at least 85%, at least 90%, or at least 95% identity to a sequence selected from the group consisting of the polynucleotides referenced in Table 14, rows 2-4599, columns H and J. In some embodiments, the gajA gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 14, rows 2-4599, columns H and J. In some embodiments, the nucleic acid sequence of a gajA gene comprises a nucleic acid sequence selected from the group consisting of the polynucleotides referenced in Table 14, rows 2-4599, columns H and J.

As used herein, the term “GajB” refers to the polynucleotide or expression product e.g., polypeptide encoded by the gajB gene. In some embodiments, the term “GajB” refers to a GajB polypeptide. In some embodiments, the product of the gajB gene comprises a pfam04257 domain. In some embodiments, the gajB gene encodes a pfam00580 domain. In some embodiments, the gajB gene encodes a pfam13361 domain. In some embodiments, the product of the gajB gene comprises a pfam04257 domain or a pfam00580 domain or a pfam13361 domain, or any combination thereof. In some embodiments, the product of the gajB gene comprises a pfam04257 domain, a pfam00580 domain, and a pfam13361 domain. In some embodiments, the gajB gene encodes a helicase. In some embodiments, the gajB gene encodes a UvrD helicase.

In some embodiments, GajB polypeptide is encoded by a gene positioned within 5-60 genes of a gene encoding a GajA polypeptide in a genome of a prokaryotic cell. In some embodiments, GajB polypeptide is encoded by a gene positioned within 5 genes upstream (5′) or downstream (3′) to a gene encoding a GajA polypeptide in a genome of a prokaryotic cell. In some embodiments, GajB polypeptide is encoded by a gene positioned within 5 genes upstream (5′) and downstream (3′) to a gene encoding a GajA polypeptide in a genome of a prokaryotic cell.

In some embodiments, a GajB polypeptide is about 598 amino acids long (median gene size).

In some embodiments, the GajB polypeptide comprises the amino acid sequence having at least 80% homology to a sequence selected from the group consisting of the polypeptides referenced in Table 14, rows 2-4599, columns L and M. In some embodiments, the GajB polypeptide comprises the amino acid sequence having at least 80% homology within similar domain regions within a sequence selected from the group consisting of the polypeptides referenced in Table 14, rows 2-4599, columns L and M. In some embodiments, the GajB polypeptide comprises a homolog of a polypeptide having the amino acid sequences set forth in the polypeptides referenced in Table 14, rows 2-4599, columns L and M.

In some embodiments, a GajB polypeptide homolog comprises a UvrD helicase domain. In some embodiments, a GajB polypeptide homolog comprises a UvrD helicase activity.

In some embodiments, the GajB polypeptide comprises the amino acid sequence having at least 80% identity to a sequence selected from the group consisting of the polypeptides referenced in Table 14, rows 2-4599, columns L and M. In some embodiments, the GajB polypeptide comprises the amino acid sequence having at least 80% identity within similar domain regions within a sequence selected from the group consisting of the polypeptides referenced in Table 14, rows 2-4599, columns L and M.

In some embodiments, the GajB polypeptide comprises an amino acid sequence having at least 80%, at least 85%, at least 90%, or at least 95% homology to a sequence selected from the group consisting of the polypeptides referenced in Table 14, rows 2-4599, columns L and M. In some embodiments, the GajB polypeptide comprises the amino acid sequence having at least 80%, 85%, 90%, 95% homology within similar domain regions within a sequence selected from the group consisting of the polypeptides referenced in Table 14, rows 2-4599, columns L and M. In some embodiments, the GajB polypeptide comprises an amino acid sequence having at least 80%, at least 85%, at least 90%, or at least 95% identity to a sequence selected from the group consisting of the polypeptides referenced in Table 14, rows 2-4599, columns L and M. In some embodiments, the GajB polypeptide comprises the amino acid sequence having at least 80%, 85%, 90%, 95% identity within similar domain regions within a sequence selected from the group consisting of the polypeptides referenced in Table 14, rows 2-4599, columns L and M. In some embodiments, the GajB polypeptide comprises an amino acid sequence selected from the group consisting of the polypeptides referenced in Table 14, rows 2-4599, columns L and M.

In some embodiments, the GajB polypeptide comprises an amino acid sequence encoded by a nucleic acid sequence having at least 80%, at least 85%, at least 90%, or at least 95% homology to a sequence selected from the group consisting of the polynucleotides referenced in Table 14, rows 2-4599, columns L and N. In some embodiments, the GajB polypeptide comprises the amino acid sequence encoded by a nucleic acid sequence having at least 80%, 85%, 90%, 95% homology within similar domain regions within a sequence selected from the group consisting of the polynucleotides referenced in Table 14, rows 2-4599, columns L and N. In some embodiments, the GajB polypeptide comprises an amino acid sequence encoded by a nucleic acid sequence having at least 80%, at least 85%, at least 90%, or at least 95% identity to a sequence selected from the group consisting of the polynucleotides referenced in Table 14, rows 2-4599, columns L and N. In some embodiments, the GajB polypeptide comprises the amino acid sequence encoded by a nucleic acid sequence having at least 80%, 85%, 90%, 95% homology within similar domain regions within a sequence selected from the group consisting of the polynucleotides referenced in Table 14, rows 2-4599, columns L and N. In some embodiments, the GajB polypeptide comprises an amino acid sequence encoded from a nucleic acid sequence selected from the group consisting of the polynucleotides referenced in Table 14, rows 2-4599, columns L and N.

In some embodiments, the nucleic acid sequence of a gajB gene comprises a nucleic acid sequence having at least 80%, at least 85%, at least 90%, or at least 95% homology to a sequence selected from the group consisting of the polynucleotides referenced in Table 14, rows 2-4599, columns L and N. In some embodiments, the gajB gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 14, rows 2-4599, columns L and N. In some embodiments, the nucleic acid sequence of a gajB gene comprises a nucleic acid sequence having at least 80%, at least 85%, at least 90%, or at least 95% identity to a sequence selected from the group consisting of the polynucleotides referenced in Table 14, rows 2-4599, columns L and N. In some embodiments, the gajB gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 14, rows 2-4599, columns L and N. In some embodiments, the nucleic acid sequence of a gajB gene comprises a nucleic acid sequence selected from the group consisting of the polynucleotides referenced in Table 14, rows 2-4599, columns L and N.

A skilled artisan would appreciate that the terms a “functional portion of a Defense System VI component” or “functional fragment of Defense System VI component” or “functional portion of a Gabija defense system component” or “functional fragment of Gabija defense system component” refers to a functional portion of a Gabija polynucleotide or polypeptide, as disclosed herein, which expression is sufficient to elicit an anti-phage activity alone or in combination with the at least one of the other Gabija polynucleotides or polypeptides disclosed herein or functional portions thereof.

The terms “GajA”, “GajB”, “gajA” and “gajB”, also refer to functional GajA, GajB, gajA and gajB, homologs, which exhibit the desired activity (i.e., conferring phage resistance). Such homologs can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide sequences references in Table 14 rows 2-4599 columns H and I and L and M, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polynucleotide sequences references in Table 14 rows 2-4599 columns H and J and L and N, respectively. In some embodiments, such homologs comprise at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% homology or identity within similar domain regions of the polypeptide sequences references in Table 14 rows 2-4599 columns H and I and L and M, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or homology within nucleotide sequences encoding similar domain regions to the polynucleotide sequences references in Table 14 rows 2-4599 columns H and J and L and N, respectively.

Table 14 presents embodiments of components of Defense System VI that may be found in a diverse array of bacteria and archaea genomes (referenced in Table 18). Table 14 presents embodiments of nucleic acid sequences encoding gene cassettes of Defense System VI (referenced in Table 18).

The Septu Defense System

In some embodiments, a defense system disclosed herein comprises a Septu anti-phage defense system (Table 15).

In some embodiments, a Septu defense system provides a host cell with resistance to entry of foreign nucleic acid invasion. In some embodiments, a host cell expressing a functional Septu defense system (Defense System VII) provides the host cell resistance foreign nucleic acid invasion.

In some embodiments, a Septu defense system provides a host cell with resistance to at least one phage.

In some embodiments, a microbial cell comprising a Septu defense system protects the microbial cell from phage infection. In some embodiments, a microbial cell expressing a Septu defense system protects the microbial cell from phage infection. In some embodiments, a microbial cell expressing a functional Septu defense system protects the microbial cell from phage infection. In some embodiments, a bacterial cell comprising a Septu defense system protects the bacterial cell from phage infection. In some embodiments, a bacterial cell expressing a Septu defense system protects the bacterial cell from phage infection. In some embodiments, a bacterial cell expressing a functional Septu defense system protects the bacterial cell from phage infection.

In some embodiments, a Septu defense system provides a host cell with resistance to plasmid transformation. In some embodiments, a Septu defense system (Defense System VII) provides a host cell with resistance to plasmid transformation. In some embodiments, a host cell expressing a functional Septu defense system (Defense System VII) provides a host cells with resistance to plasmid transformation.

In some embodiments, a Septu defense system (Defense system VII) provides a host cell with resistance to entry of conjugative elements. In some embodiments, a host cell expressing a function Septu defense system (Defense System VII) provides the host cell resistance from entry of conjugative elements.

As used herein, the term “a Septu anti-phage defense system” may be used interchangeably with the term “a Defense System VII”, having all the same meanings and qualities. A skilled artisan would appreciate that the term “Septu system” may be used interchangeably in some embodiments with “Septu defense system”, “Septu the defense system”, “Septu anti-phage system”, and “Defense System VII”, having all the same meanings and qualities.

In some embodiments, a microbial species does not comprise an endogenous Defense System VII. In some embodiments, a microbial species does not express an endogenous Defense System VII. In some embodiments, a microbial species does not express an endogenous functional Defense System VII.

In some embodiments, a bacterial species does not comprise an endogenous Defense System VII. In some embodiments, a bacterial species does not express an endogenous Defense System VII. In some embodiments, a bacterial species does not express an endogenous functional Defense System VII.

A Septu defense system comprises, in some embodiments, a nucleic acid construct comprising a nucleic acid sequence encoding a PtuA polypeptide comprising a pfam13304 domain or a pfam02463 domain or a combination thereof, and a PtuB polypeptide comprising pfam13395 domain or a pfam01844 domain or a combination thereof. In some embodiments, a Septu defense system comprises a nucleic acid construct comprising a nucleic acid sequence comprising a ptuA gene and a ptuB gene.

In some embodiments, a Septu defense system comprises, a PtuA polypeptide comprising a pfam13304 domain or a pfam02463 domain or a combination thereof, and a PtuB polypeptide comprising pfam13395 domain or a pfam01844 domain or a combination thereof. In some embodiments, a Septu defense system comprising anti-phage activity comprises a PtuA polypeptide and a PtuB polypeptide.

In some embodiments, a Defense System VII comprises a PtuA polypeptide comprising a pfam13304 domain or a pfam02463 domain or a combination thereof, or comprising an amino acid sequence having at least 80% identity to a sequence selected from the group referenced in Table 15 rows 2-12507 columns H and I; or a PtuB polypeptide comprising pfam13395 domain or a pfam 01844 domain or a combination thereof, or comprising an amino acid sequence having at least 80% identity to a sequence selected from the group referenced in Table 15 rows 2-2507 columns L and M; or combination thereof.

In some embodiments, a Defense System VII comprises at least two different polypeptides selected from a PtuA polypeptide comprising a pfam13304 domain or a pfam02463 domain or a combination thereof, or comprising an amino acid sequence having at least 80% identity to a sequence selected from the group referenced in Table 15 rows 2-12507 columns H and I; and a PtuB polypeptide comprising pfam13395 domain or a pfam 01844 domain or a combination thereof, or comprising an amino acid sequence having at least 80% identity to a sequence selected from the group referenced in Table 15 rows 2-2507 columns L and M.

In some embodiments, a Septu defense system having an anti-phage activity comprises a nucleic acid construct comprising a nucleic acid sequence encoding a PtuA polypeptide and a PtuB polypeptide. In some embodiments, a Septu defense system having an anti-phage activity comprises a nucleic acid construct comprising a nucleic acid sequence encoding a PtuA polypeptide comprising an AAA domain (“ATPases Associated with diverse cellular Activities” domain) or a RecF/RecN/SMC N-terminal domain (a domain found at the N terminus of structural maintenance of chromosomes (SMC) proteins) or an N-terminal HNH nuclease domain, or any combination thereof, and a PtuB polypeptide comprising a HNH endonuclease activity.

In some embodiments, a Septu defense system having an anti-phage activity comprises a nucleic acid construct comprising a nucleic acid sequence encoding a PtuA polypeptide comprising an AAA domain (“ATPases Associated with diverse cellular Activities” domain). In some embodiments a PtuA polypeptide comprises a RecF/RecN/SMC N-terminal domain (a domain found at the N terminus of structural maintenance of chromosomes (SMC) proteins). In some embodiments, a PtuA polypeptide comprises an N-terminal HNH nuclease domain. In some embodiments, a PtuA polypeptide comprises an AAA domain (“ATPases Associated with diverse cellular Activities” domain) and a RecF/RecN/SMC N-terminal domain (a domain found at the N terminus of structural maintenance of chromosomes (SMC) proteins) and an N-terminal HNH nuclease domain. In some embodiments, a Septu defense system having an anti-phage activity comprises a nucleic acid construct comprising a nucleic acid sequence encoding a PtuB polypeptide comprising a HNH endonuclease. In some embodiments, a Septu defense system having an anti-phage activity comprises a nucleic acid construct comprising a nucleic acid sequence encoding a PtuA polypeptide comprising an AAA domain (“ATPases Associated with diverse cellular Activities” domain) and a RecF/RecN/SMC N-terminal domain (a domain found at the N terminus of structural maintenance of chromosomes (SMC) proteins). In some embodiments, a Septu defense system having an anti-phage activity comprises a nucleic acid construct comprising a nucleic acid sequence encoding a PtuA polypeptide comprising an AAA domain (“ATPases Associated with diverse cellular Activities” domain) and a RecF/RecN/SMC N-terminal domain (a domain found at the N terminus of structural maintenance of chromosomes (SMC) proteins) and an N-terminal HNH nuclease domain, and a PtuB polypeptide comprising a HNH endonuclease. In some embodiments, a Septu defense system having an anti-phage activity comprises a nucleic acid construct comprising a nucleic acid sequence encoding a PtuA polypeptide comprising an AAA domain (“ATPases Associated with diverse cellular Activities” domain) and a RecF/RecN/SMC N-terminal domain (a domain found at the N terminus of structural maintenance of chromosomes (SMC) proteins), and a PtuB polypeptide comprising a HNH endonuclease.

In some embodiments, the Septu anti-phage defense system comprises a nucleic acid construct comprising nucleic acid sequences encoding a cassette comprising ptuA and ptuB genes.

In some embodiments, a Septu (Defense System VII) gene cassette comprises the nucleic acid sequence:

(SEQ ID NO: 12; Table 4 construct 42)

| | |
|---|---|
| 1 | gggaatgcaa aaaggaggcg ggacttgagt gcgagttctg cctctttttt gctgaattta |
| 61 | gaaatctttt aagttttatc taaaaaagca ttcactttat tgttatttat gtagtaagag |
| 121 | taaaatttta ttaaaaattc taaagctgta ttggtttata agctagtgct atatctacta |
| 181 | tttgttctgg agtaagatct tcatcattaa aagaaaggca ttattatttt tggtgaatgt |
| 241 | tatttagtat aaaccatagg ggaatggaat attttatgat aaaattgagt ggagtaaata |
| 301 | attgtaaaga taaataaacg gatagcatgt aaaaaaaggg ttgaccttga gccttattaa |
| 361 | gacgggtgtt gtagtaataa atgaattaag agaaaaacat ttgtaattag tcaacaaaaa |
| 421 | gggagttgct tgaatgttag ttataaaaga attaaaaatt gatgggattg gcggtattaa |
| 481 | tagccttaaa cttaatttta acgaggggtt aaatttgatt tgcggtccaa atggtgtagg |
| 541 | gaaaaccaca attcttgaaa gtattggcca tatgtttagt aatggtagtc gtagtaaggt |
| 601 | taagagaaat gttaaatttg atcaggggag ttgtgaaatt agctattcaa cattcctaga |
| 661 | tactatacat acacagtcat gtattttaag gaattatgaa gaaaatgatt cgctggattg |
| 721 | gcatggtgga aatgcaaatt tggctaaaga ggtcattgtc ttcaaagctc aaagatcatt |
| 781 | cacttatacg caattagatt ctcttcgtaa agatacggaa acaagtgatg gtaaatttct |
| 841 | ggatgattct atgaatggaa ttcaattttt cgactttaag aattggttta tccatcgttt |
| 901 | tttatttagt catgtagata atgaattaac aacagtacaa aaagagaatt ttaaacttgc |
| 961 | gacagattgt tttggtattt tagataatag cattcgtttt agtagtgtta aaagtgatac |
| 1021 | attcgatata attataagta cctcaaatgg tgaaatctat tttgagtatc tatcatcagg |
| 1081 | attcaaatca tgtatctata ttattcatgg gttaattaaa gaaatagagt atcgttttaa |
| 1141 | aaacgatggt ggtattaaag ttcaagatta tgaagggcta atactaatcg atgagttgga |
| 1201 | ccttcattta catccacagt ggcaagcgaa aatgatttat ttaataaagc atatattacc |
| 1261 | taaagctcaa attattgcaa caacacatag tcctcatatg gtacaagctg cagcaattaa |
| 1321 | tgaacttata gctttaggaa tcgatgaaga tgcggatgta tatgtaagac aactacctaa |
| 1381 | tgttcaatat ggatttcagg gatggactgt tgaggaaata ttggaagacg ttatggggtt |
| 1441 | agatgaaaca cggtctccaa tgtacataga ggctataact gaatttgaaa aatctctaga |
| 1501 | tgaagaaaat gaggagaaga tatttgaagc ttattataag ttagatttaa tgctacaccc |
| 1561 | aagtaatcca cttcgcaagt tgttaaagtt acaagtagct caattcggga gggttataga |
| 1621 | gtgattcatt taaaacgccc taatagtcct agtaaactta taaatgaaga agcaaaacta |
| 1681 | actcaagaat ttcttcaaaa tggtaatagt gtttggggaa agccttatat taagcttgct |
| 1741 | cttctagaga tgtcaaataa caaatgtgta tactgtgaat gtagactaga tgaagagagt |
| 1801 | aagtatatgg aggtagagca ctttctacca aaagatactt atcctaatct agttgtcaat |
| 1861 | tggagaaatt tattaccaag ttgtaaaaga tgtaatggaa aaaaagggac acatgattca |
| 1921 | aaaaaagaac ctattatcaa tcctactgtt gatactccaa gtaatcatat aaaaatgttt |
| 1981 | aactattgcc taaagggaaa agatttaaaa gggaaaacaa cagttgatgt attgcaccta |
| 2041 | aaccaacttg atagattagt atacccaaga atgttaattg gaacaaaaac tatagaaaca |
| 2101 | gtagaaaaac ttttagaaat ggcagagaac tttaatttga atattaatgc tactgggcgt |
| 2161 | atcaaaagtc gtattataca tggcactaca cagttgctca tagaagctca accgcattcc |
| 2221 | gaatattctg cgacagttgc ctcggtttta tttaacaatg aagattttgt taagttaaaa |
| 2281 | gagattatga ttaagtgcac aatctggaat acagaacatc aaaagttact gtctaatgca |
| 2341 | gaaagaaata ttttggtaga gccactttac aattgcctca ataataagaa ttaatgattc |
| 2401 | taacgtatac tctagctgaa agtatatagt tagtgtattc cttggggtag cttcaaccta |
| 2461 | tttgatatta gatgtagaat tagagttaca attgatgtga aagtaagtgt caatggttca |
| 2521 | caaattataa agtacacaac agtaaaggta agccatacta tgtaacagca aatgaggtct |
| 2581 | atgtatatgg gaagtaaaaa aagcggtctg ctcaaattat gagtagaccg cttttttact |
| 2641 | taagtatgct atttatcact tcataaatat aaaattacat atcaattact tttttacgac |
| 2701 | tttaccggca acacctgttt accgaattag ttgatgaaga ctcatcaatt aattcaatgc |
| 2761 | ttttatttgc aatatattgc ctcgcgttct tattaaaatc aatcagtgta gagaaaagaa |
| 2821 | gctggaagag ttagagggga tatatgatgt gtttgtgaag ggatatataa tagaggaaat |
| 2881 | ataaaaagtg ctaacaagtt tgcaaacgaa cttgctaaca cctatacaga atagacataa |
| 2941 | aatatcgctg atatgtcatc tttgaaatag ctttaatcag ttaaatcaca cttatacgaa |
| 3001 | atgcttatag taacagtctt aatatcaaga atataatcat ccagatattc ctttactttt |
| 3061 | cagacgaaaa cgagggatga tat. |

The coding regions for each of the ptuA and ptuB gene sequences within this embodiment of a Septu cassette (SEQ ID NO: 12) are as follows: nucleotides 434-1624 encode an embodiment of a PtuA polypeptide, and nucleotides 1621-2394 encode an embodiment of a PtuB polypeptide.

In some embodiments, a Defense System VII comprising a PtuA polypeptide and a PtuB polypeptide is encoded by the nucleic acid sequence set forth in SEQ ID NO: 12. In some embodiments, a Defense System VII comprising a PtuA polypeptide and a PtuB polypeptide, is encoded by a nucleic acid sequence having at least 80% homology to the sequence set forth in SEQ ID NO: 12. In some embodiments, a Defense System VII comprising a PtuA polypeptide and a PtuB polypeptide, is encoded by a nucleic acid sequence having at least 85% homology, or 90% homology, or 95% homology, or 97% homology, or 98% homology, or 99% homology to the sequence set forth in SEQ ID NO: 12. In some embodiments, a Defense System VII comprising a PtuA polypeptide and a PtuB polypeptide, is encoded by a nucleic acid sequence having at least 80% identity to the sequence set forth in SEQ ID NO: 12. In some embodiments, a Defense System VII comprising a PtuA polypeptide and a PtuB polypeptide, is encoded by a nucleic acid sequence having at least 85% identity, or 90% identity, or 95% identity, or 97% identity, or 98% identity, or 99% identity to the sequence set forth in SEQ ID NO: 12.

In some embodiments, a Septu (Defense System VII) gene cassette comprises the nucleic acid sequence:

(SEQ ID NO: 13; construct 43 of Table 4)

| | |
|---|---|
| 1 | ggctttttaa gtgtacattt ttattgaaga ttggatagaa ttcaacaaga tgaatttgat |
| 61 | gtttattaaa cataagatgg aaaggctatg tgttaaataa aaagatgaaa taattaccat |
| 121 | tagtgtataa aataataagt aactagtttg gtaatgattt taaggatatg aagtatatta |
| 181 | accgtgacat attatagttg aaattcagtt ttatatcatg tgaaaatatg cttttgagga |
| 241 | gggtaaagat atgattcgaa tacataaaga taaatatttt gattcaatat atgatgaatt |
| 301 | gttagaacaa gcaactcaag aaaagtatat ggatgagtat ttactagaaa agtttatgaa |
| 361 | aagatacttt aatggtaaat gttcatactg tgaaattcga atgaaagagt tacgactaac |
| 421 | aattgacttt tatagaccta taaatggatc gttaaacacg atggatggtg aatttcatcc |
| 481 | tgatcattat aaatggttaa aaaatgaaga tgataatata ctttcaattt gtgtagaatg |
| 541 | taagcgagcg aaaagtaatc gttttccagt agatggcgat gtcgcttcta ttaatgctga |
| 601 | taagaataaa ttagtgaaag aaagacgttt attgatacat ccttgtagag attatcctga |
| 661 | aaggcatttt gattacgatg aaagtggaat ggtttattat aaaagtaaaa aaggcgaggt |
| 721 | tactgtagat gttttaaatt tgaatcgcgg tatgttggta gaaatgcgtg cccaagtata |
| 781 | tagtgaattt aataatcttt gttatttatt ctcaatggag caaagtccct actatttaaa |
| 841 | gcaaattcta caagaagttc agttggattc aatatttatt ggtttaaaaa agtttatact |
| 901 | ttctgaatgg gtaatcttaa atgagaaaat tccatttctt caagaatttg agccattatt |
| 961 | aggtaagaga ttgcaggaag aagaagtgag agttttatca gttagaaata ataagattag |
| 1021 | tcaaattgat attgataata atatccttta tgttgataat gataagcgga gaaagcgtgt |
| 1081 | tcctgcaagg aaaattaatg attattacga tgtatccgac gagaacgctt tagaaaaata |
| 1141 | ttatggaaaa cagcgtttta ttgaaaaaat tgaaatttat aactttaaaa gtattagaaa |
| 1201 | tatgaaaatt gattttactc tcagtaaaag cagtaatgct ccttggctaa tgttattagg |
| 1261 | agaaaatggt gtgggcaaaa gctcaatttt acaagcaatt gcattaacac taatgggaaa |
| 1321 | cgaacaacgg caaaaaatta taaaaaagaa accttatgaa tatttaacaa aaggctttga |
| 1381 | tgaaggttct ataaagataa aattatcagg catgcaagag cctattagta tttatttaaa |
| 1441 | ttcgaattca tttgaattta caggtgaaaa tcatcaaagg cctagggttc taattttagg |
| 1501 | ttatggctca acgcgattac ttcctagaga agaaatgtta tcaaattata aggtgacttg |
| 1561 | ggcaagaata gaaaatctat ttaatccttt tattccatta gttaatgtaa gagagtatct |
| 1621 | cttatcttta aataatgaag attttaataa tgttaaaaaa gctattgagt ccttattttt |
| 1681 | agatgaagta ataattgatc gggatcagat atatgaagaa gtatattttg gatttgcgaa |
| 1741 | ttcttacagt aaactagaag atttaagtga tggctatcag acaattattg ctctggctac |
| 1801 | tgatattatg atggttatga agaatagatg gcgaaatttt gatgctgaag gaattgtttt |
| 1861 | aatagatgaa attgatgcac atcttcatcc aaggtggaat atagaaattg tttctcgatt |
| 1921 | aaaaaatgct tttccgaaaa ttcaatttat tgcaactacc cataacccgc tatcacttcg |
| 1981 | tggtttaata gatggagaag ttgcagtatt actagaaaat gaggaaagag aagcgtatat |
| 2041 | tactcaaaag ttaccttctc aaaaaggatt taatgtagaa ggattattaa cttctaaatt |
| 2101 | ttttggattg tatgacacca tgccagattt aaatgaatta tttgatcgat attatttact |
| 2161 | tttatctaat cctagtccta atgaaaggca aaaggaagaa ataaaaaatt tacaagataa |
| 2221 | attgtcaaaa tatgaaaaag taggtaccac attaagagaa caaaaattct atgaagcagt |
| 2281 | ggattactat tttgcacagt acagaaaaaa taatgtggaa ttaagtgatg aagagtttaa |
| 2341 | caatattata gaagatgcta ttaaatattt tgaaaggtaa gattttttat gataaaagtt |
| 2401 | caaagagaag cagaaccagc tgtattaaat ctaactgact cagattctat tggttttaaa |
| 2461 | gaattggaag cggcaaaaaa aactactttt acaaaagata cgaaatttcc gttcgaggca |
| 2521 | tataaggatg aaagtgtaaa aaatctttta aaaaaaatgt ttaatggtaa atgtggatat |
| 2581 | tgtgaaagta ttattaatgt gacttcgtat gaagaaattg aacattttag acctaaaaaa |
| 2641 | gctattaata tagaaggtat acaaggattg acttatcctg gttactattg gttggctatg |
| 2701 | agttggaata atttgttgat ttcttgtcaa aggtgcaata ggtcgcataa aaaaaattat |
| 2761 | tttccaatag aaaacgaaag taatcgtgca aaagctcccg gtgaagaaag tggagaggag |
| 2821 | gtattactat taaacccttg tgaagatgac ccgagtgaac atttggaatt taaggataca |
| 2881 | ggtattattg aatttaaaga gggatctaaa aagggagaaa aatctataaa agtttatgct |
| 2941 | cttcatagga gagaacttac tgaagaaaga gcgaaagttg ctaaggatat tgagctgaag |
| 3001 | aaagtacaaa tacttgacgg attaagtact ctaaaggtgc ttttacgcta tcaagaagat |
| 3061 | agtgatcttc aaaaagagat tgaaaaaact gtatcaaata taatatcatt atatgatttt |
| 3121 | atatttgaat atgaaaatga tcctaacagg ccttatcagg caatggtaac tcagattact |
| 3181 | tcggattttc tgagtgaacg taaagaatta atcaataaat tgaagacaac atccaatcgg |
| 3241 | aaaaattcct gtgaacaaag tcatatttca agttaagtta atttaggata agagctataa |
| 3301 | aaaagctctt atcctattta ttatgaagtt tagcgagata gtaaaagtaa cataattaat |
| 3361 | atttaaaaaa ttattttttc ttgagtaacg gtattaactc tgctacattt aatatttttt |
| 3421 | gtctttataa aatgccttat gtcaattata aaaaaatcat gatgctttct aatttagtcc |
| 3481 | tagcgtcttc tttaaattaa gatactataa atatgaggtc cctcaatgct gttgtttata |
| 3541 | aagtattttt gctaactgga ttactttgtt gttacgttca tctgacttag tgttccagct |
| 3601 | ccacctaact caattaatat ttctatgttt atcatcagtc tatcttttta aaattagtat |
| 3661 | atctaaattt ttgcaatgga agcaattggt tttatataat tgtagaagga tttaccaatg |
| 3721 | gacaatagcg ggaggattca aaaaa. |

The coding regions for each of the ptuA and ptuB gene sequences within this embodiment of a Septu cassette (SEQ ID NO: 13) are as follows: nucleotides 251-2380 encode an embodiment of a PtuA polypeptide, and nucleotides 2389-3276 encode an embodiment of a PtuB polypeptide.

In some embodiments, a Defense System VII comprising a PtuA polypeptide and a PtuB polypeptide is encoded by the nucleic acid sequence set forth in SEQ ID NO: 13. In some embodiments, a Defense System VII comprising a PtuA polypeptide and a PtuB polypeptide, is encoded by a nucleic acid sequence having at least 80% homology to the sequence set forth in SEQ ID NO: 13. In some embodiments, a Defense System VII comprising a PtuA polypeptide and a PtuB polypeptide, is encoded by a nucleic acid sequence having at least 85% homology, or 90% homology, or 95% homology, or 97% homology, or 98% homology, or 99% homology to the sequence set forth in SEQ ID NO: 13. In some embodiments, a Defense System VII comprising a PtuA polypeptide and a PtuB polypeptide, is encoded by a nucleic acid sequence having at least 80% identity to the sequence set forth in SEQ ID NO: 13. In some embodiments, a Defense System VII comprising a PtuA polypeptide and a PtuB polypeptide, is encoded by a nucleic acid sequence having at least 85% identity, or 90% identity, or 95% identity, or 97% identity, or 98% identity, or 99% identity to the sequence set forth in SEQ ID NO: 13.

In some embodiments, a construct comprising the Septu defense system encodes one component of the defense system, whereby multiple constructs, for example but not limited to, two constructs may be used to assemble the functional defense system. In some embodiments, the components of a Septu defense system comprise genes ptuA and ptuB. In some embodiments, the components of a Septu defense system consist of genes ptuA and ptuB. In some embodiments, the components of a Septu defense system comprise nucleic acid sequences encoding a PtuA polypeptide and a PtuB polypeptide. In some embodiments, the components of a Septu defense system consist of nucleic acid sequences encoding a PtuA polypeptide and a PtuB polypeptide.

In some embodiments, a construct comprising the Septu defense system encodes one component of the defense system, whereby a second construct may be used to assemble the functional defense system. In some embodiments, a construct comprising the Septu defense system encodes one component of the defense system, whereby additional constructs may be used to assemble the functional defense system. For example, in some embodiments each of PtuA and PtuB may be encoded by nucleic acid sequences comprised in different constructs, wherein expression of the combination of constructs produces a functional Septu defense system.

In some embodiments, the components making up a functional Septu anti-phage defense system comprise a PtuA polypeptide and a PtuB polypeptide, each encoded by a ptuA and a ptuB gene, respectively. In some embodiments, a ptuA gene encodes a polypeptide comprising an AAA domain (“ATPases Associated with diverse cellular Activities” domain). In some embodiments, a ptuA gene encodes a polypeptide comprising a RecF/RecN/SMC N-terminal domain (a domain found at the N terminus of structural maintenance of chromosomes (SMC) proteins). In some embodiments, a ptuA gene encodes a polypeptide comprising an N-terminal HNH nuclease domain. In some embodiments, a ptuA gene encodes a polypeptide comprising a AAA domain (“ATPases Associated with diverse cellular Activities” domain) or a RecF/RecN/SMC N-terminal domain (a domain found at the N terminus of structural maintenance of chromosomes (SMC) proteins) or an N-terminal HNH nuclease domain, or any combination thereof. In some embodiments, a ptuA gene encodes a polypeptide comprising a AAA domain (“ATPases Associated with diverse cellular Activities” domain) and a RecF/RecN/SMC N-terminal domain (a domain found at the N terminus of structural maintenance of chromosomes (SMC) proteins). In some embodiments, a ptuA gene encodes a polypeptide comprising a AAA domain (“ATPases Associated with diverse cellular Activities” domain) and a RecF/RecN/SMC N-terminal domain (a domain found at the N terminus of structural maintenance of chromosomes (SMC) proteins) and an N-terminal HNH nuclease domain.

In some embodiments, a ptuB gene encodes a polypeptide comprising a HNH endonuclease.

In some embodiments, a Septu defense system having an anti-phage activity comprises a nucleic acid encoding a PtuA polypeptide. In some embodiments, a Septu defense system having an anti-phage activity comprises a nucleic acid encoding a PtuA polypeptide and a PtuB polypeptide. In some embodiments, a Septu defense system having an anti-phage activity comprises a nucleic acid encoding a PtuB polypeptide.

In some embodiments, a Septu defense system having an anti-phage activity comprises a nucleic acid comprising a ptuA gene. In some embodiments, a Septu defense system having an anti-phage activity comprises a nucleic acid comprising a ptuA gene and a ptuB gene. In some embodiments, a Septu defense system having an anti-phage activity comprises a nucleic acid comprising ptuB gene.

In some embodiments, a Septu defense system having an anti-phage activity comprises a nucleic acid construct comprising nucleic acids encoding polypeptides in a set order. In one embodiment, the 5′ to 3′ order of polypeptides encoded is PtuA and PtuB. In some embodiments the 5′ to 3′ order of polypeptides does not affect the anti-phage activity. In some embodiments the 5′ to 3′ order of polypeptides does affect the anti-phage activity.

In some embodiments, the 5′ to 3′ order of polypeptides is random, for example any order of nucleic acid sequences encoding PtuA and PtuB.

In some embodiments, a Septu defense system having an anti-phage activity comprises a nucleic acid construct comprising genes in a set order. In some embodiment, the 5′ to 3′ order of genes is ptuA and ptuB. In some embodiment, the 5′ to 3′ order of genes in a Septu defense system is not ptuA and ptuB. In some embodiments the 5′ to 3′ order of genes does not affect the anti-phage activity. In some embodiments the 5′ to 3′ order of genes does affect the anti-phage activity.

In some embodiments, the 5′ to 3′ order of genes is random, for example any order of ptuA and ptuB.

In some embodiments, the Septu system (Defense System VII) composition and order is as shown in FIG. 3B.

In some embodiments, a Septu defense system having an anti-phage activity originates from a microbial genome, for example a bacterial or an archaeal genome (Table 15). A skilled artisan would appreciate that the Septu system is not present in the majority of bacteria and or archaea species.

In some embodiments, a functional Septu defense system comprises a construct comprising nucleic acid sequences encoding polypeptides, wherein the nucleic acid sequence encoding each polypeptide may originate from a different microbial species. For example, the source of the nucleic acid sequence encoding a PtuA polypeptide may be one microbial species, while the source of the nucleic acid sequence encoding a PtuB polypeptide may be a different microbial species. In some embodiments, a functional Septu defense system comprises a non-naturally occurring combination of polypeptide components. In some embodiments, a functional Septu defense system comprises a combination of at least two polypeptides that do not naturally occur together.

In some embodiments, a functional Septu defense system comprises a construct comprising nucleic acid sequences encoding polypeptides, wherein the nucleic acid sequence encoding each polypeptide may originate from a different bacterial species. For example, the source of the nucleic acid sequence encoding PtuA polypeptide may be one bacterial species, while the source of the nucleic acid sequence encoding a PtuB polypeptide may be a different bacterial species.

In some embodiments, the source of the nucleic acid encoding a PtuA polypeptide and a PtuB polypeptide is the same. In some embodiments, the source of the nucleic acid encoding a PtuA polypeptide and a PtuB polypeptide is the not the same. In some embodiments, the source of the nucleic acid sequence of any of the components of a Septu defense system comprises any of the species listed in Table 15.

In some embodiments, a Septu defense system having an anti-phage activity comprises a nucleic acid construct comprising non-coding regions between the nucleic acid sequences encoding the polypeptides. In some embodiments, the non-coding regions between the nucleic acid sequences comprises nucleic acid sequence not naturally occurring in the microbial species of origin. In some embodiments, the non-coding regions between the nucleic acid sequences comprises nucleic acid sequence is the naturally occurring in the microbial species of origin.

In some embodiments, a Septu system disclosed herein comprises a multi-gene phage resistance system broadly distributed in microbial genomes, for example but not limited to bacteria and archaea. According to some embodiments, the Septu system components are located in a gene cluster (a cassette of genes) in a microbial cell genome. According to some embodiments, the Septu system components are located in close proximity (e.g. positioned within 20 genes, 10 genes, 5 genes or less one from the other) in a genome of a prokaryotic cell. According to some embodiments the prokaryotic cell expresses an endogenous Septu defense system. According to some embodiments, the prokaryotic cell expresses an endogenous functional Septu defense system. According to some embodiments, the species of prokaryotic cell is selected from the group consisting of the species listed in Table 15. According to some embodiments a prokaryotic cell expresses a non-endogenous Septu defense system. According to some embodiments, a prokaryotic cell expresses a non-endogenous functional Septu defense system. According to some embodiments, the species of prokaryotic cell expressing a non-endogenous functional Septu defense system is selected from the group consisting of the species listed in Table 15.

In some embodiments, Septu defense system components comprise PtuA and PtuB polypeptides. In some embodiments, Septu defense system components comprise functional portions of PtuA and PtuB polypeptides. In some embodiments, the Septu defense system functional components are encoded by ptuA and ptuB genes.

Non-limiting embodiments of endogenous Septu systems and the respective location of their components are provided in Table 15 herein.

In some embodiments, the components of a Septu system may be identified by the presence of similar domains present within each component. In some embodiments, domains comprise pfam domains and or clusters of orthologous groups (COG) domains.

In some embodiments, the term “PtuA” refers to the polynucleotide or expression product e.g., the polypeptide encoded by the ptuA gene. In some embodiments, the term “PtuA” refers to a PtuA polypeptide. In some embodiments, the ptuA gene encodes a polypeptide comprising a pfam13304 domain. In some embodiments, the ptuA gene encodes a polypeptide comprising a pfam02463 domain. In some embodiments, the ptuA gene encodes a polypeptide comprising a pfam13304 domain and a pfam02463 domain. In some embodiments, the PtuA polypeptide comprises a pfam13304 domain. In some embodiments, the PtuA polypeptide comprises a pfam02463 domain. In some embodiments, the PtuA polypeptide comprises a pfam13304 domain and a pfam02463 domain.

In some embodiments, PtuA polypeptide is encoded by a gene positioned within 5-60 genes of a gene encoding a PtuB polypeptide in a genome of a prokaryotic cell. In some embodiments, PtuA polypeptide is encoded by a gene positioned within 5 genes upstream (5′) or downstream (3′) to a gene encoding a PtuB polypeptide in a genome of a prokaryotic cell. In some embodiments, PtuA polypeptide is encoded by a gene positioned within 5 genes upstream (5′) and downstream (3′) to a gene encoding a PtuB polypeptide in a genome of a prokaryotic cell.

In some embodiments, PtuA and PtuB are encoded by genes positioned sequentially 5′ to 3′ in a genome of a prokaryotic cell. In some embodiments, PtuA and PtuB are encoded by genes positioned contiguously 5′ to 3′ in a genome of a prokaryotic cell.

In some embodiments, ptuA and ptuB genes are positioned sequentially 5′ to 3′ in a genome of a prokaryotic cell. In some embodiments, ptuA and ptuB, genes are positioned contiguously 5′ to 3′ in a genome of a prokaryotic cell.

As used herein, the term “PtuA” refers to the polynucleotide or expression product e.g., polypeptide encoded by the ptuA gene. In some embodiments, the term “PtuA” refers to a PtuA polypeptide. In some embodiments, the product of the ptuA gene comprises a pfam13304 domain. In some embodiments, the ptuA gene encodes a pfam02463 domain. In some embodiments, the product of the ptuA gene comprises a pfam13304 domain or a pfam02463 domain, or any combination thereof. In some embodiments, the product of the ptuA gene comprises a pfam13304 domain and a pfam02463 domain. In some embodiments, the ptuA gene encodes a AAA domain. In some embodiments, the ptuA gene encodes a RecF/RecN/SMC N-terminal domain (a domain found at the N terminus of structural maintenance of chromosomes (SMC) proteins). In some embodiments, the ptuA gene encodes an N-terminal HNH nuclease domain. In some embodiments, the ptuA gene encodes a AAA domain, or a RecF/RecN/SMC N-terminal domain (a domain found at the N terminus of structural maintenance of chromosomes (SMC) proteins), or an N-terminal HNH nuclease domain, or any combination thereof. In some embodiments, the ptuA gene encodes a AAA domain and a RecF/RecN/SMC N-terminal domain (a domain found at the N terminus of structural maintenance of chromosomes (SMC) proteins) and an N-terminal HNH nuclease domain. In some embodiments, the ptuA gene encodes a AAA domain and a RecF/RecN/SMC N-terminal domain (a domain found at the N terminus of structural maintenance of chromosomes (SMC) proteins). In some embodiments, the ptuA gene encodes a RecF/RecN/SMC N-terminal domain (a domain found at the N terminus of structural maintenance of chromosomes (SMC) proteins) and an N-terminal HNH nuclease domain.

In some embodiments, PtuA polypeptide is encoded by a gene positioned within 5-60 genes of a gene encoding a PtuB polypeptide in a genome of a prokaryotic cell. In some embodiments, PtuA polypeptide is encoded by a gene positioned within 5 genes upstream (5′) or downstream (3′) to a gene encoding a PtuB polypeptide in a genome of a prokaryotic cell. In some embodiments, PtuA polypeptide is encoded by a gene positioned within 5 genes upstream (5′) and downstream (3′) to a gene encoding a PtuB polypeptide in a genome of a prokaryotic cell.

In some embodiments, a PtuA polypeptide is about 452 amino acids long (median gene size).

In some embodiments, the PtuA polypeptide comprises the amino acid sequence having at least 80% homology to a sequence selected from the group consisting of the polypeptides referenced in Table 15, rows 2-2507, columns H and I. In some embodiments, the PtuA polypeptide comprises the amino acid sequence having at least 80% homology to similar domain regions in a sequence selected from the group consisting of the polypeptides referenced in Table 15, rows 2-2507, columns H and I. In some embodiments, a PtuA polypeptide homologue comprises an AAA domain (“ATPases Associated with diverse cellular Activities” domain). In some embodiments, a PtuA polypeptide homologue comprises a RecF/RecN/SMC N-terminal domain (a domain found at the N terminus of structural maintenance of chromosomes (SMC) proteins). In some embodiments, a PtuA polypeptide homologue comprises an N-terminal HNH nuclease domain. In some embodiments, a PtuA polypeptide homologue comprises an AAA domain (“ATPases Associated with diverse cellular Activities” domain) and a RecF/RecN/SMC N-terminal domain (a domain found at the N terminus of structural maintenance of chromosomes (SMC) proteins). In some embodiments, a PtuA polypeptide homologue comprises an AAA domain (“ATPases Associated with diverse cellular Activities” domain) and a RecF/RecN/SMC N-terminal domain (a domain found at the N terminus of structural maintenance of chromosomes (SMC) proteins) and an HNH nuclease domain. In some embodiments, the PtuA polypeptide comprises a homolog of a polypeptide having the amino acid sequences set forth in the polypeptides referenced in Table 15, rows 2-2507, columns H and I.

In some embodiments, the PtuA polypeptide comprises the amino acid sequence having at least 80% identity to a sequence selected from the group consisting of the polypeptides referenced in Table 15, rows 2-2507, columns H and I. In some embodiments, the PtuA polypeptide comprises the amino acid sequence having at least 80% homology to a sequence selected from the group consisting of the polypeptides referenced in Table 15, rows 2-2507, columns H and I. In some embodiments, the PtuA polypeptide comprises the amino acid sequence having at least 80% homology to similar domain regions in a sequence selected from the group consisting of the polypeptides referenced in Table 15, rows 2-2507, columns H and I. In some embodiments, the PtuA polypeptide comprises the amino acid sequence having at least 80% identity to similar domain regions in a sequence selected from the group consisting of the polypeptides referenced in Table 15, rows 2-2507, columns H and I.

In some embodiments, the PtuA polypeptide comprises an amino acid sequence having at least 80%, at least 85%, at least 90%, or at least 95% homology to a sequence selected from the group consisting of the polypeptides referenced in Table 15, rows 2-2507, columns H and I. In some embodiments, the PtuA polypeptide comprises the amino acid sequence having at least 80%, at least 85%, at least 90%, or at least 95%, homology to similar domain regions in a sequence selected from the group consisting of the polypeptides referenced in Table 15, rows 2-2507, columns H and I. In some embodiments, the PtuA polypeptide comprises an amino acid sequence having at least 80%, at least 85%, at least 90%, or at least 95% identity to a sequence selected from the group consisting of the polypeptides referenced in Table 15, rows 2-2507, columns H and I. In some embodiments, the PtuA polypeptide comprises the amino acid sequence having at least 80%, at least 85%, at least 90%, at least 95% identity to similar domain regions in a sequence selected from the group consisting of the polypeptides referenced in Table 15, rows 2-2507, columns H and I. In some embodiments, the PtuA polypeptide comprises an amino acid sequence selected from the group consisting of the polypeptides referenced in Table 15, rows 2-2507, columns H and I.

In some embodiments, the PtuA polypeptide comprises an amino acid sequence encoded by a nucleic acid sequence having at least 80%, at least 85%, at least 90%, or at least 95% homology to a sequence selected from the group consisting of the polynucleotides referenced in Table 15, rows 2-2507, columns H and J. In some embodiments, the PtuA polypeptide comprises the amino acid sequence encoded by a nucleic acid sequence having at least 80%, at least 85%, at least 90%, or at least 95%, homology to similar domain regions in a sequence selected from the group consisting of the polynucleotides referenced in Table 15, rows 2-2507, columns H and J. In some embodiments, the PtuA polypeptide comprises an amino acid sequence encoded by a nucleic acid sequence having at least 80%, at least 85%, at least 90%, or at least 95% identity to a sequence selected from the group consisting of the polynucleotides referenced in Table 15, rows 2-2507, columns H and J. In some embodiments, the PtuA polypeptide comprises the amino acid sequence encoded by a nucleic acid sequence having at least 80%, at least 85%, at least 90%, or at least 95%, homology to similar domain regions in a sequence selected from the group consisting of the polynucleotides referenced in Table 15, rows 2-2507, columns H and J. In some embodiments, the PtuA polypeptide comprises an amino acid sequence encoded from a nucleic acid sequence selected from the group consisting of the polynucleotides referenced in Table 15, rows 2-2507, columns H and J.

In some embodiments, the nucleic acid sequence of a ptuA gene comprises a nucleic acid sequence having at least 80%, at least 85%, at least 90%, or at least 95% homology to a sequence selected from the group consisting of the polynucleotides referenced in Table 15, rows 2-2507, columns H and J. In some embodiments, the nucleic acid sequence of a ptuA gene comprises a nucleic acid sequence having at least 80%, at least 85%, at least 90%, or at least 95% homology within regions encoding similar domain sequences to a sequence selected from the group consisting of the polynucleotides referenced in Table 15, rows 2-2507, columns H and J.

In some embodiments, the nucleic acid sequence of a ptuA gene encodes a polypeptide comprising an AAA domain (“ATPases Associated with diverse cellular Activities” domain). In some embodiments, the nucleic acid sequence of a ptuA gene encodes a polypeptide comprising a RecF/RecN/SMC N-terminal domain (a domain found at the N terminus of structural maintenance of chromosomes (SMC) proteins). In some embodiments, the nucleic acid sequence of a ptuA gene encodes a polypeptide comprising an HNH nuclease domain. In some embodiments, the nucleic acid sequence of a ptuA gene encodes a polypeptide comprising an AAA domain (“ATPases Associated with diverse cellular Activities” domain) and a RecF/RecN/SMC N-terminal domain (a domain found at the N terminus of structural maintenance of chromosomes (SMC) proteins). In some embodiments, the nucleic acid sequence of a ptuA gene encodes a polypeptide comprising an AAA domain (“ATPases Associated with diverse cellular Activities” domain) and a RecF/RecN/SMC N-terminal domain (a domain found at the N terminus of structural maintenance of chromosomes (SMC) proteins) and an HNH nuclease domain. In some embodiments, the nucleic acid sequence of a ptuA gene comprises a nucleic acid sequence having at least 80%, at least 85%, at least 90%, or at least 95% identity to a sequence selected from the group consisting of the polynucleotides referenced in Table 15, rows 2-2507, columns H and J. In some embodiments, the nucleic acid sequence of a ptuA gene comprises a nucleic acid sequence having at least 80%, at least 85%, at least 90%, or at least 95% identity within regions encoding similar domain sequences to a sequence selected from the group consisting of the polynucleotides referenced in Table 15, rows 2-2507, columns H and J. In some embodiments, the nucleic acid sequence of a ptuA gene comprises a nucleic acid sequence selected from the group consisting of the polynucleotides referenced in Table 15, rows 2-2507, columns H and J.

As used herein, the term “PtuB” refers to the polynucleotide or expression product e.g., polypeptide encoded by the ptuB gene. In some embodiments, the term “PtuB” refers to a PtuB polypeptide. In some embodiments, the product of the ptuB gene comprises a pfam13395 domain. In some embodiments, the ptuB gene encodes a pfam01844 domain. In some embodiments, the product of the ptuB gene comprises a pfam13395 domain or a pfam01844 domain, or any combination thereof. In some embodiments, the product of the ptuB gene comprises a pfam13395 domain and a pfam01844 domain. In some embodiments, the ptuB gene encodes a HNH endonuclease.

In some embodiments, PtuB polypeptide is encoded by a gene positioned within 5-60 genes of a gene encoding a PtuA polypeptide in a genome of a prokaryotic cell. In some embodiments, PtuB polypeptide is encoded by a gene positioned within 5 genes upstream (5′) or downstream (3′) to a gene encoding a PtuA polypeptide in a genome of a prokaryotic cell. In some embodiments, PtuB polypeptide is encoded by a gene positioned within 5 genes upstream (5′) and downstream (3′) to a gene encoding a PtuA polypeptide in a genome of a prokaryotic cell.

In some embodiments, a PtuB polypeptide is about 219 amino acids long (median gene size).

In some embodiments, the PtuB polypeptide comprises an amino acid sequence having at least 80% homology to a sequence selected from the group consisting of the polypeptides referenced in Table 15, rows 2-2507, columns L and M. In some embodiments, the PtuB polypeptide comprises the amino acid sequence having at least 80% homology to similar domain regions in a sequence selected from the group consisting of the polypeptides referenced in Table 15, rows 2-2507, columns L and M. In some embodiments, a homologue of the PtuB polypeptide comprises an HNH endonuclease. In some embodiments, the PtuB polypeptide comprises an amino acid sequence having at least 80% identity to a sequence selected from the group consisting of the polypeptides referenced in Table 15, rows 2-2507, columns L and M. In some embodiments, the PtuB polypeptide comprises the amino acid sequence having at least 80% identity to similar domain regions in a sequence selected from the group consisting of the polypeptides referenced in Table 15, rows 2-2507, columns L and M.

In some embodiments, the PtuB polypeptide comprises an amino acid sequence having at least 80%, at least 85%, at least 90%, or at least 95% homology to a sequence selected from the group consisting of the polypeptides referenced in Table 15, rows 2-2507, columns L and M. In some embodiments, the PtuB polypeptide comprises the amino acid sequence having at least 80%, at least 85%, at least 90%, or at least 95%, homology to similar domain regions in a sequence selected from the group consisting of the polypeptides referenced in Table 15, rows 2-2507, columns L and M. In some embodiments, the PtuB polypeptide comprises an amino acid sequence having at least 80%, at least 85%, at least 90%, or at least 95% identity to a sequence selected from the group consisting of the polypeptides referenced in Table 15, rows 2-2507, columns L and M. In some embodiments, the PtuB polypeptide comprises the amino acid sequence having at least 80%, at least 85%, at least 90%, or at least 95%, identity to similar domain regions in a sequence selected from the group consisting of the polypeptides referenced in Table 15, rows 2-2507, columns L and M. In some embodiments, the PtuB polypeptide comprises an amino acid sequence selected from the group consisting of the polypeptides referenced in Table 15, rows 2-2507, columns L and M.

In some embodiments, the PtuB polypeptide comprises an amino acid sequence encoded from a nucleic acid sequence having at least 80%, at least 85%, at least 90%, or at least 95% homology to a sequence selected from the group consisting of the polynucleotides referenced in Table 15, rows 2-2507, columns L and N. In some embodiments, the PtuB polypeptide comprises the amino acid sequence encoded by a nucleic acid sequence having at least 80%, at least 85%, at least 90%, or at least 95%, homology to similar domain regions in a sequence selected from the group consisting of the polynucleotides referenced in Table 15, rows 2-2507, columns L and N. In some embodiments, the PtuB polypeptide comprises an amino acid sequence encoded from a nucleic acid sequence having at least 80%, at least 85%, at least 90%, or at least 95% identity to a sequence selected from the group consisting of the polynucleotides referenced in Table 15, rows 2-2507, columns L and N. In some embodiments, the PtuB polypeptide comprises the amino acid sequence encoded by a nucleic acid sequence having at least 80%, at least 85%, at least 90%, or at least 95%, identity to similar domain regions in a sequence selected from the group consisting of the polynucleotides referenced in Table 15, rows 2-2507, columns L and N. In some embodiments, the PtuB polypeptide comprises an amino acid sequence encoded from a nucleic acid sequence selected from the group consisting of the polynucleotides referenced in Table 15, rows 2-2507, columns L and N.

In some embodiments, the nucleic acid sequence of a ptuB gene comprises a nucleic acid sequence having at least 80%, at least 85%, at least 90%, or at least 95% homology to a sequence selected from the group consisting of the polynucleotides referenced in Table 15, rows 2-2507, columns L and N. In some embodiments, the nucleic acid sequence of a ptuB gene comprises a nucleic acid sequence having at least 80%, at least 85%, at least 90%, or at least 95% identity to a sequence selected from the group consisting of the polynucleotides referenced in Table 15, rows 2-2507, columns L and N. In some embodiments, the nucleic acid sequence of a ptuB gene comprises a nucleic acid sequence having at least 80%, at least 85%, at least 90%, or at least 95% homology within the nucleotide sequence encoding similar domain regions to regions with a sequence selected from the group consisting of the polynucleotides referenced in Table 15, rows 2-2507, columns L and N. In some embodiments, the nucleic acid sequence of a ptuB gene comprises a nucleic acid sequence having at least 80%, at least 85%, at least 90%, or at least 95% identity to a sequence encoding similar domain regions selected from the group consisting of the polynucleotides referenced in Table 15, rows 2-2507, columns L and N. In some embodiments, the ptuB gene homolog encodes a polypeptide comprising a HNH endonuclease. In some embodiments, the nucleic acid sequence of a ptuB gene comprises a nucleic acid sequence selected from the group consisting of the polynucleotides referenced in Table 15, rows 2-2507, columns L and N.

A skilled artisan would appreciate that the terms a “functional portion of a Defense System VII component” or “functional fragment of Defense System VII component” or “functional portion of a Septu defense system component” or “functional fragment of Septu defense system component” refers to a functional portion of a Septu polynucleotide or polypeptide, as disclosed herein, which expression is sufficient to elicit an anti-phage activity alone or in combination with the at least one of the other Septu polynucleotides or polypeptides disclosed herein or functional portions thereof.

The terms “PtuA”, “PtuB”, “ptuA” and “ptuB”, also refer to functional PtuA, PtuB, ptuA and ptuB, homologs, which exhibit the desired activity (i.e., conferring phage resistance). Such homologs can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide sequences referenced in Table 15 rows 2-2507 columns H and I and L and M, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polynucleotide sequences referenced in Table 15 rows 2-2507 columns H and J and L and N, respectively. In some embodiments, such homologs comprise at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% homology or identity within similar domain regions of the polypeptide sequences referenced in Table 15 rows 2-2507 columns H and I and L and M, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or homology within nucleotide sequences encoding similar domain regions to the polynucleotide sequences referenced in Table 15 rows 2-2507 columns H and J and L and N, respectively.

Table 15 presents embodiments of components of Defense System VII that may be found in a diverse array of bacteria and archaea genomes (referenced in Table 18). Table 15 presents embodiments of nucleic acid sequences encoding gene cassettes of Defense System VII (referenced in Table 18).

The Lamassu Defense System

In some embodiments, a defense system disclosed herein comprises a Lamassu anti-phage defense system. (Table 16)

In some embodiments, a Lamassu defense system provides a host cell with resistance to entry of foreign nucleic acid invasion. In some embodiments, a host cell expressing a functional Lamassu defense system (Defense System VIII) provides the host cell resistance foreign nucleic acid invasion.

In some embodiments, a Lamassu defense system provides a host cell with resistance to at least one phage.

In some embodiments, a microbial cell comprising a Lamassu defense system protects the microbial cell from phage infection. In some embodiments, a microbial cell expressing a Lamassu defense system protects the microbial cell from phage infection. In some embodiments, a microbial cell expressing a functional Lamassu defense system protects the microbial cell from phage infection. In some embodiments, a bacterial cell comprising a Lamassu defense system protects the bacterial cell from phage infection. In some embodiments, a bacterial cell expressing a Lamassu defense system protects the bacterial cell from phage infection. In some embodiments, a bacterial cell expressing a functional Lamassu defense system protects the bacterial cell from phage infection.

In some embodiments, a Lamassu defense system provides a host cell with resistance to a plasmid.

As used herein, the term “a Lamassu anti-phage defense system” may be used interchangeably with the term “a Defense System VII”, having all the same meanings and qualities. A skilled artisan would appreciate that the term “Lamassu system” may be used interchangeably in some embodiments with “Lamassu defense system”, “Lamassu the defense system”, “Lamassu anti-phage system”, and “Defense System VIII”, having all the same meanings and qualities.

In some embodiments, a Lamassu defense system (Defense System VIII) provides a host cell with resistance to plasmid transformation. In some embodiments, a host cell expressing a functional Lamassu defense system (Defense System VIII) provides a host cells with resistance to plasmid transformation.

In some embodiments, a Lamassu defense system (Defense system VIII) provides a host cell with resistance to entry of conjugative elements. In some embodiments, a host cell expressing a function Lamassu defense system (Defense System VIII) provides the host cell resistance from entry of conjugative elements.

In some embodiments, a microbial species does not comprise an endogenous Defense System VIII. In some embodiments, a microbial species does not express an endogenous Defense System VIII. In some embodiments, a microbial species does not express an endogenous functional Defense System VIII.

In some embodiments, a bacterial species does not comprise an endogenous Defense System VIII. In some embodiments, a bacterial species does not express an endogenous Defense System VIII. In some embodiments, a bacterial species does not express an endogenous functional Defense System VIII.

A Lamassu defense system comprises, in some embodiments, a nucleic acid construct comprising a nucleic acid sequence encoding a LmuA polypeptide comprising a pfam14130 domain or a DUF4297 domain or a combination thereof, and a LmuB polypeptide comprising a pfam02463 domain. In some embodiments, a Lamassu defense system comprises a nucleic acid construct comprising a nucleic acid sequence comprising a lmuA gene and a lmuB gene.

In some embodiments, a Lamassu defense system comprises, a LmuA polypeptide comprising a pfam14130 domain or a DUF4297 domain or a combination thereof, and a LmuB polypeptide comprising a pfam02463 domain. In some embodiments, a Lamassu defense system comprises a LmuA polypeptide and a LmuB polypeptide.

In some embodiments, a Defense System VIII comprises a LmuA polypeptide comprising a pfam14130 domain or a DUF4297 domain or a combination thereof, or comprising an amino acid sequence having at least 80% identity to a sequence selected from the group referenced in Table 16 rows 2-698 columns H and I; or a LmuB polypeptide comprising a pfam02463 domain or comprising an amino acid sequence having at least 80% identity to a sequence selected from the group referenced in Table 16 rows 2-698 columns L and M; or a combination thereof.

In some embodiments, a Defense System VIII comprises at least two different polypeptide components selected from a LmuA polypeptide comprising a pfam14130 domain or a DUF4297 domain or a combination thereof, or comprising an amino acid sequence having at least 80% identity to a sequence selected from the group referenced in Table 16 rows 2-698 column H and I; and a LmuB polypeptide comprising a pfam02463 domain or comprising an amino acid sequence having at least 80% identity to a sequence selected from the group referenced in Table 16 rows 2-698 columns L and M.

In some embodiments, a Lamassu defense system having an anti-phage activity comprises a nucleic acid construct comprising a nucleic acid sequence encoding a LmuA polypeptide and a LmuB polypeptide. In some embodiments, a Lamassu defense system having an anti-phage activity comprises a nucleic acid construct comprising a nucleic acid sequence encoding a LmuA polypeptide and a LmuB polypeptide comprising a RecF/RecN/SMC N-terminal domain.

In some embodiments, a Lamassu defense system having an anti-phage activity comprises a nucleic acid construct comprising a nucleic acid sequence encoding a LmuB polypeptide comprising a RecF/RecN/SMC N-terminal domain.

In some embodiments, the Lamassu anti-phage defense system comprises a nucleic acid construct comprising nucleic acid sequences encoding a cassette comprising lmuA and lmuB genes.

In some embodiments, a Lamassu (Defense System VIII) gene cassette comprises the nucleic acid sequence:

(SEQ ID NO: 7; construct 13 of Table 4)

| | |
|---|---|
| 1 | tgcatattta taattgtgtg ttgaggcgag tgttaatcta ttgcaaatag ttttgcaatt |
| 61 | ctccactagt caattatatc tcttgatatc catcttgata aaaaactttt aaagacactt |
| 121 | tctcatttgg gaggtgtctt tatttattgg tatcatagtt tttttatggg agctaataaa |
| 181 | agtaacataa gatccgctca tgcattctgt gtctgataat gaaaaaaatt aggtatttat |
| 241 | atttgatagt tcaaaatcta tattattgaa aacataataa aattttcctg ataaatagta |
| 301 | ccattttttt gaaaatcctg atatcatata aataagagag aggatatcag gaggagcgca |
| 361 | atgattgaaa ttgacaacgg gggagccatc gcactaaaag gatttaacta tcaaaaagct |
| 421 | tccatcatac ttgtaatgat tcataatttt gaaaaagata attttatagt catacctgaa |
| 481 | tcgcaagaag attttgaaat tcatcttgga caagatactt attttataca agtgaaagga |
| 541 | acaaaaaaac tttccatagg taagttgaag tcaaggccta gcggaaaagc ttcaatcata |
| 601 | gagaaaaatc tttctcctgg taatgttgga gatataagga aaatatttct ctgggatata |
| 661 | gcggagttaa caaaaaacga attgataagc caagaaggaa cgcttattcc tatgaaacac |
| 721 | agcctttcat tgaaacaaaa gacagagatt ataaatactc ttgatttaga tgaagaacag |
| 781 | aaaaatagaa tgaacaatca atatatatat ataacccctt ttccaaacga tataaatttg |
| 841 | gctttaacat ttttaaaagg agaaatggtt aatgaaaact tgctggtttc taacgatcga |
| 901 | gctaaattag ttttaggaga actatcattg gaaatagatc gtaaaagtga aatagtagtt |
| 961 | tctactgaaa gtgatgtaga aaggaaaaaa atcgacggga attatcttaa gcaggtattc |
| 1021 | attaatatta agcaaaaaga gatgtttgat gaaattttag ataatctctc tatcaacaca |
| 1081 | attatgaaaa aaaaagtaaa gaaggagaaa ttaagaatac cactcttgta tcaaaatatt |
| 1141 | aaagagcaaa cgaagcaaaa ggcagatatt aatttactta tgagggagaa tgatgagggt |
| 1201 | gctattaact atctcagaga tctattggta gagatagtgc cagatatgaa gcctacagaa |
| 1261 | ctatctattg ctttagcaat agactgtttt tgtgagttgg gagaataaaa tctatgataa |
| 1321 | taattgcttt ttctattctt gattttaaaa ataaagaagc acaaaatttt gactttaaag |
| 1381 | ctgggactaa tctaattgtt agtaaaggaa ataccaaagg taaatcaagt cttttaaaat |
| 1441 | caatgtatta tacattagga tttgatgtgc atcaatttcc tagtaactgg aacataaatt |
| 1501 | ttatgtattt ccaaatagaa gtcttaataa ataatgttaa atataatata actagacaaa |
| 1561 | aaaatatttt tagagtaagt gatgtagaag tacctttgaa tgtaaaagag tattcagagt |
| 1621 | ggcttcaaca taaacttgag attaaaatgc agttagctaa tactcatact aagcacttat |
| 1681 | acgaggcata ttcttcggca gtaattttac cattttacat agatcaagac gattcttggg |
| 1741 | atggcggaat ttatagaaat gtaactaata ctctgaatca atatactcgc ataccggcag |
| 1801 | atattttcaa aagtgttttt aatctctcca attacgaact tctggagttg caaaattcgc |
| 1861 | ttactaatta tagtaaagaa aagaatacgg tagtgtctac aataaagagt ttattaaatg |
| 1921 | ttttggaaga ttatagacat gaaaacgcag atgtccctac tgtttcaaaa attgataaaa |
| 1981 | tagccttaaa taaagatatc gatcgctact tgcagatgca aaatgaatta aatgaacaaa |
| 2041 | tagtaaaata taaaatgaag ctattaaaca agcaagaaat gttggattta caaaagcaag |
| 2101 | aattgtctga attagagcag cttttaaaaa tgaacaaaaa gagatataac tctattgaaa |
| 2161 | cagaatgtca atattgtcat tctaaattaa caaaagaaca atctttaaca agacttgatt |
| 2221 | tatcgaataa ttattttgaa atatcgcttt tgaaagagga aatagaaaaa gaagtagtaa |
| 2281 | aattgactaa tgaaattata atattcgaat cacaacaaaa ttctattgaa tctaaaattg |
| 2341 | atgaaattca tcgtaggatt caaaattcta aagatctact tacgattgat gattatgtaa |
| 2401 | aagcaactgc taaaaaagaa gcatcaaatg aattggaaag tttagttgat aaacaagttc |
| 2461 | tttctaaata taatttagaa gaaaaaatta aagtattaag aagggaaatt aataaactga |
| 2521 | agaaagagaa agaaagttta agagagatta ttgagcgaga ttacactgat ttggtatttg |
| 2581 | aaataaaaaa agtgttgaat gatttaaatg atacaaagct ggatttatct gaattaaatc |
| 2641 | tggatgaact taaatttctt gaatttaaga aaatatcggg gagtggtatg gataaaaata |
| 2701 | aaaaattttt agcttactat ttaatttatt ttagtttgtt gagaaagtat agctcttata |
| 2761 | taattccctt ttgtatggat tcgtttatta aaaatgaaat tacgggtgag actgcaaaaa |
| 2821 | agatgtttga agcaatagaa aaatattttt ttgacactaa tcagtctttc ttttcaatcg |
| 2881 | tttctgaaaa ccttaaacat ttagagtttg tagatagtta caataagatt aatgttgagg |
| 2941 | gtaagctgtt ggttagagat aaatatgatg agattgcttt gaaattcaaa tttgatagtt |
| 3001 | aaaattatta aagggccttg aatgacattt gatagttcaa actttcaaat gcctgaagag |
| 3061 | aaaaatgatt atccgaaaga aattaactca tatagaatta tgcacttaat attcttaatt |
| 3121 | ttttagtaga atactcaatc ctttgatttt ttttggaata taataacatt tttcatcaga |
| 3181 | aaacaacaag gggaaaagcc aatttcttta cagtaggagt tggctttatt gtgcagaaac |
| 3241 | taaattaaat attatatgtt ttttcgatct taaccatatg aaaggataaa taaacaaaga |
| 3301 | gttgattgac ttcgttcttc gcttcataga ttgaggagtt gattcaactt tccattct |

The coding regions for each of the lmuA and lmuB gene sequences within this embodiment of a Lamassu cassette (SEQ ID NO: 7) are as follows: nucleotides 361-1308 encode an embodiment of a LmuA polypeptide, and nucleotides 1314-3002 encode an embodiment of a LmuB polypeptide.

In some embodiments, a Defense System VIII comprising a LmuA polypeptide and a LmuB polypeptide is encoded by the nucleic acid sequence set forth in SEQ ID NO: 7. In some embodiments, a Defense System VIII comprising a LmuA polypeptide and a LmuB polypeptide, is encoded by a nucleic acid sequence having at least 80% homology to the sequence set forth in SEQ ID NO: 7. In some embodiments, a Defense System VIII comprising a LmuA polypeptide and a LmuB polypeptide, is encoded by a nucleic acid sequence having at least 85% homology, or 90% homology, or 95% homology, or 97% homology, or 98% homology, or 99% homology to the sequence set forth in SEQ ID NO: 7. In some embodiments, a Defense System VIII comprising a LmuA polypeptide and a LmuB polypeptide, is encoded by a nucleic acid sequence having at least 80% identity to the sequence set forth in SEQ ID NO: 7. In some embodiments, a Defense System VIII comprising a LmuA polypeptide and a LmuB polypeptide, is encoded by a nucleic acid sequence having at least 85% identity, or 90% identity, or 95% identity, or 97% identity, or 98% identity, or 99% identity to the sequence set forth in SEQ ID NO: 7.

In some embodiments, a Lamassu (Defense System VIII) gene cassette comprises the nucleic acid sequence:

(SEQ ID NO: 8; construct 14 of Table 4)

| | |
|---|---|
| 1 | gaagatgcgg ttaacaaaat agaagagatt ttgttggtaa tgtgaccaat tttgtgatca |
| 61 | ctggttttgt atagtatagg aatagaaaaa taacaaccgt atcaaacgtt aatttgacac |
| 121 | ggtttctaca actatgccct cggagaggct cgaactcatg gccttcatcc tgtcaaagtg |
| 181 | taaatagaag tcgttagttt aacgtaagtc aaatcggaaa tcggcactat ttacggatac |
| 241 | cgttatatca acgtttgcag ctaaatagtt atacgtccgt cttgttttct attattaatt |
| 301 | aaagcgtgta gtaccgccat actgcaagat ataaaaccgt taattgtcat tttaggatcc |
| 361 | ttcacattca ttgattatta cgaagctgca ggaatggcta tgtaaacagt acatgtttgg |
| 421 | aaagaaatga agatgatagg agaaaatctt catgaaattt atcatcgagc gaattaaatg |
| 481 | gacgtatgta aaaggtgatg aacaacccac gatgaaaatt aaatacaagt aaacgagcca |
| 541 | cgctcgtcac tgggcgtggt tttaatgtgt gtaattggaa actgtatgta aaaaagaaat |
| 601 | taacaagttt gttagtactg gatgatttag gataaaaaat gaaatttata acaaattata |
| 661 | gcttaaaatt gatataatat gataagaagg atgttttgct attgtaattt aagaaacaag |
| 721 | aattttgaac agaaaaaatt cactatataa atgaaattgt aaaattgcct ttgggattta |
| 781 | aaggagggat ggctagtgga tataagttct tatcctagct tggaaactta tgaagcacgt |
| 841 | gagaatacag gttcaaggtc ttcaaacaga ttagctatgc aaattagttt tgctatgtgg |
| 901 | aagatttttg aattatatca aaatgaagat tttactgtgg ctatggattg tattgatgat |
| 961 | gtagtcatat ttaaaacatc aaatgaaaat ccaactattg tgacttatca attgaaaaca |
| 1021 | aaggatgcta ctacggggaa ctttgaactg agaaagctaa ttggtgataa tgtatttttg |
| 1081 | aaaatgtatg atcacattga aaaaatagat gcagatgttg aagaaatata tttaataact |
| 1141 | aacaatcctt tgaaatttag aaagaaaacg gttaatgcag aacggatttt atttgaaaaa |
| 1201 | ttagatgaag atataaaaga gttaattgaa gaaaatatgt ctcagagtaa agtttttgca |
| 1261 | gataaaggat tatcatctaa gtttatatat tctttggtag atatgtcttt tcataatcat |
| 1321 | agagaaatca gtcaatataa attaaattct ttattaatga aagaaaaaat agatattagt |
| 1381 | ataacagctg cagatgcact ttttaatgca ttgcaagata ttttaacaac taagcaaaat |
| 1441 | tatgagttta gtttacaaga tgaatttaat acggtactaa agaaaaaaag ttactcgaaa |
| 1501 | acggaattta catcactatt ggataactcg aaaaaaatta atgcctatgt tttatctttc |
| 1561 | gaggatattc gtaccaacta taaaagcaaa cctttaaagc tgaaagaaga aagtttatac |
| 1621 | aggagagcga tggctagtgt taaagaaaaa tgcaataaat ctcctaatat attacaaaat |
| 1681 | ataaatgaag atatttttca atatgctaaa gagcagattg atatcaatga cgaaataacg |
| 1741 | agaattgagc tagtcatact tttacaaagt gtttttgacg acaagataaa tgttgagtta |
| 1801 | tctaaagaag aaaaagaaat tctatatatg caaaatatcg aattagccct aagggaggga |
| 1861 | taaactcatg aataaaataa taattcgtaa gttatttata tttgatgtga ttgaaaaaaa |
| 1921 | agccaaaagt gtagattttg aagatggtat caatattgtt acaagtaaag gaaatcaact |
| 1981 | gggtaaatca actataatga aatctattta ttatacattg ggtgcagaag tgttttttgc |
| 2041 | cgacagatta aatgtaaaga gtaaaatcca tatgttagag acagacgtta atgataagaa |
| 2101 | atataccttt attagacatg gcgatgttgt tgtgataaaa gatggtaaag gaatttttaa |
| 2161 | gacgtcgaac gcttctgaat tatcatctaa gttgcatgat atatttggat ttagcgtttt |
| 2221 | tctagaagat aaacagaaaa aatatgtgat agctccacct gtatttcgtt acattcctta |
| 2281 | ttatattgac caagaccatg gttggacatc agaattgaaa tcttttgata aattaggtca |
| 2341 | attcgataaa aaaagcaggg atttattatt ttattatcat ttaaatattc ttgatgaaga |
| 2401 | ttatggagta aagttgaaag agaaaaaaga actagatgct tcaatgactg atttgaaaac |
| 2461 | taggaaaaaa gagattttag gcctactcgc atacataaga gagaatatta cagcttttaa |
| 2521 | tttagaaatg gatattacag cgttacaaat tcaaaagaga gaaatactta ataaatataa |
| 2581 | aaagtattca tatgatttga ataacatacg aagaaaaatt ttagaatatc aagaagaaat |
| 2641 | atttaaaatt gacaatgtaa ttgataattt aaatagtact ttgaaacaaa atgataaagt |
| 2701 | aagagaacat ataaaacatc aatttgatgt agagtgcccg tattgtaata atcattttga |
| 2761 | aatacaggcg aaagatatat taagaattaa ttataatatt gtagacttag aggcatcaaa |
| 2821 | attagaaatg ctagatataa aagaaaaatt attggggaaa atgaaaaaag tacaaaaaga |
| 2881 | gtatgaagat tatcaagcta cgttgaaagc tattgaagaa gaaaaagttg attcagaaaa |
| 2941 | tacacttgaa gacatactga aatttaaagg gttacaagaa actcaaaatc aactaaatac |
| 3001 | ggaacttgtc aaaaatactg gtgatataga agaaaagtca gaagacttaa aagaaataag |
| 3061 | acgagattta aaaaaatggc aggatgaaat agataaagtt aacagtaggt ataaggatat |
| 3121 | cttaaattta aatctaatta gatttaatac gaatgaacat gcactccctg aaaaatataa |
| 3181 | tattggaaaa aacctcaaag catctggtag cggtcaagtt cgagttaatt tagctagagt |
| 3241 | atattcattt ataaagcttc ttgaagaata taatcctaca ggattaaaat atccattagt |
| 3301 | aattgactct cctaaaggcg gagaacagag tactacaaat agtgaattga ttttaagatt |
| 3361 | gttgacggag aaagcgcaaa tttcaaatca aattatttta gctacaattg attttgaaag |
| 3421 | tttttataat ggagatacta aaaaattcaa cataatctct ttagaaaatg aaccttatca |
| 3481 | tttattatct gctgaagatt atcaaaataa tcaagtgata attgatgact ttgtttcttt |
| 3541 | atattttgaa gcaaatcaat aaatagaaat agtaaaagga cgctttaata cgaagcgtcc |
| 3601 | tatttaattt gaaggagccc tcaaacaggg aatcctt. |

The coding regions for each of the lmuA and lmuB gene sequences within this embodiment of a Lamassu cassette (SEQ ID NO: 8) are as follows: nucleotides 877-1863 encode an embodiment of a LmuA polypeptide, and nucleotides 1868-3562 encode an embodiment of a LmuB polypeptide.

In some embodiments, a Defense System VIII comprising a LmuA polypeptide and a LmuB polypeptide is encoded by the nucleic acid sequence set forth in SEQ ID NO: 8. In some embodiments, a Defense System VIII comprising a LmuA polypeptide and a LmuB polypeptide, is encoded by a nucleic acid sequence having at least 80% homology to the sequence set forth in SEQ ID NO: 8. In some embodiments, a Defense System VIII comprising a LmuA polypeptide and a LmuB polypeptide, is encoded by a nucleic acid sequence having at least 85% homology, or 90% homology, or 95% homology, or 97% homology, or 98% homology, or 99% homology to the sequence set forth in SEQ ID NO: 8. In some embodiments, a Defense System VIII comprising a LmuA polypeptide and a LmuB polypeptide, is encoded by a nucleic acid sequence having at least 80% identity to the sequence set forth in SEQ ID NO: 8. In some embodiments, a Defense System VIII comprising a LmuA polypeptide and a LmuB polypeptide, is encoded by a nucleic acid sequence having at least 85% identity, or 90% identity, or 95% identity, or 97% identity, or 98% identity, or 99% identity to the sequence set forth in SEQ ID NO: 8.

In some embodiments, a construct comprising the Lamassu defense system encodes one component of the defense system, whereby multiple constructs, for example but not limited to, two constructs may be used to assemble the functional defense system. In some embodiments, the components of a Lamassu defense system comprise genes lmuA and lmuB. In some embodiments, the components of a Lamassu defense system consist of genes lmuA and lmuB. In some embodiments, the components of a Lamassu defense system comprise nucleic acid sequences encoding a LmuA polypeptide and a LmuB polypeptide. In some embodiments, the components of a Lamassu defense system consist of nucleic acid sequences encoding a LmuA polypeptide and a LmuB polypeptide.

In some embodiments, a construct comprising the Lamassu defense system encodes one component of the defense system, whereby a second construct may be used to assemble the functional defense system. In some embodiments, a construct comprising the Lamassu defense system encodes one component of the defense system, whereby additional constructs may be used to assemble the functional defense system. For example, in some embodiments each of LmuA and LmuB may be encoded by nucleic acid sequences comprised in different constructs, wherein expression of the combination of constructs produces a functional Lamassu defense system.

In some embodiments, the components making up a functional Lamassu anti-phage defense system comprise a LmuA polypeptide and a LmuB polypeptide, each encoded by a lmuA and a lmuB gene, respectively.

In some embodiments, a lmuB gene encodes a polypeptide comprising a RecF/RecN/SMC N-terminal domain. In some embodiments, a LmuB polypeptide comprises a RecF/RecN/SMC N-terminal domain.

In some embodiments, a Lamassu defense system having an anti-phage activity comprises a nucleic acid encoding a LmuA polypeptide. In some embodiments, a Lamassu defense system having an anti-phage activity comprises a nucleic acid encoding a LmuA polypeptide and a LmuB polypeptide. In some embodiments, a Lamassu defense system having an anti-phage activity comprises a nucleic acid encoding a LmuB polypeptide.

In some embodiments, a Lamassu defense system having an anti-phage activity comprises a nucleic acid comprising a lmuA gene. In some embodiments, a Lamassu defense system having an anti-phage activity comprises a nucleic acid comprising a lmuA gene and a lmuB gene. In some embodiments, a Lamassu defense system having an anti-phage activity comprises a nucleic acid comprising lmuB gene.

In some embodiments, a Lamassu defense system having an anti-phage activity comprises a nucleic acid construct comprising nucleic acids encoding polypeptides in a set order. In one embodiment, the 5′ to 3′ order of polypeptides encoded is LmuA and LmuB. In some embodiments the 5′ to 3′ order of polypeptides does not affect the anti-phage activity. In some embodiments the 5′ to 3′ order of polypeptides does affect the anti-phage activity.

In some embodiments, the 5′ to 3′ order of polypeptides is random, for example any order of nucleic acid sequences encoding LmuA and LmuB.

In some embodiments, a Lamassu defense system having an anti-phage activity comprises a nucleic acid construct comprising genes in a set order. In some embodiment, the 5′ to 3′ order of genes is lmuA and lmuB. In some embodiment, the 5′ to 3′ order of genes in a Lamassu defense system is not lmuA and lmuB. In some embodiments the 5′ to 3′ order of genes does not affect the anti-phage activity. In some embodiments the 5′ to 3′ order of genes does affect the anti-phage activity.

In some embodiments, the 5′ to 3′ order of genes is random, for example any order of lmuA and lmuB.

In some embodiments, the Lamassu system (Defense System VIII) composition and order is as shown in FIG. 3B.

In some embodiments, a Lamassu defense system having an anti-phage activity originates from a microbial genome, for example a bacterial or an archaeal genome (Table 16). A skilled artisan would appreciate that the Lamassu system is not present in the majority of bacteria and or archaea species.

In some embodiments, a functional Lamassu defense system comprises a construct comprising nucleic acid sequences encoding polypeptides, wherein the nucleic acid sequence encoding each polypeptide may originate from a different microbial species. For example, the source of the nucleic acid sequence encoding a LmuA polypeptide may be one microbial species, while the source of the nucleic acid sequence encoding a LmuB polypeptide may be a different microbial species. In some embodiments, a functional Lamassu defense system comprises a non-naturally occurring combination of polypeptide components. In some embodiments, a functional Lamassu defense system comprises a combination of at least two polypeptides that do not naturally occur together.

In some embodiments, a functional Lamassu defense system comprises a construct comprising nucleic acid sequences encoding polypeptides, wherein the nucleic acid sequence encoding each polypeptide may originate from a different bacterial species. For example, the source of the nucleic acid sequence encoding a LmuA polypeptide may be one bacterial species, while the source of the nucleic acid sequence encoding a LmuB polypeptide may be a different bacterial species.

In some embodiments, the source of the nucleic acid encoding a LmuA polypeptide and a LmuB polypeptide is the same. In some embodiments, the source of the nucleic acid encoding a LmuA polypeptide and a LmuB polypeptide is the not the same. In some embodiments, the source of the nucleic acid sequence of any of the components of a Lamassu defense system comprises any of the species listed in Table 16.

In some embodiments, a Lamassu defense system having an anti-phage activity comprises a nucleic acid construct comprising non-coding regions between the nucleic acid sequences encoding the polypeptides. In some embodiments, the non-coding regions between the nucleic acid sequences comprises nucleic acid sequence not naturally occurring in the microbial species of origin. In some embodiments, the non-coding regions between the nucleic acid sequences comprises nucleic acid sequence is the naturally occurring in the microbial species of origin.

In some embodiments, a Lamassu system disclosed herein comprises a multi-gene phage resistance system broadly distributed in microbial genomes, for example but not limited to bacteria and archaea. A skilled artisan would appreciate that the majority of bacteria and archaea do not comprise a Lamassu defense system.

According to some embodiments, the Lamassu defense system components are located in a gene cluster (a cassette of genes) in a microbial cell genome. According to some embodiments, the Lamassu system components are located in close proximity (e.g. positioned within 20 genes, 10 genes, 5 genes or less one from the other) in a genome of a prokaryotic cell. According to some embodiments the prokaryotic cell expresses an endogenous Lamassu defense system. According to some embodiments, the prokaryotic cell expresses an endogenous functional Lamassu defense system. According to some embodiments, the species of prokaryotic cell is selected from the group consisting of the species listed in Table 16.

According to some embodiments a prokaryotic cell expresses a non-endogenous Lamassu defense system. According to some embodiments, a prokaryotic cell expresses a non-endogenous functional Lamassu defense system. According to some embodiments, the species of prokaryotic cell expressing a non-endogenous functional Lamassu defense system is selected from the group consisting of the species listed in Table 16.

In some embodiments, Lamassu defense system components comprise LmuA and LmuB polypeptides. In some embodiments, Lamassu defense system components comprise functional portions of LmuA and LmuB polypeptides. In some embodiments, the Lamassu defense system functional components are encoded by lmuA and lmuB genes.

Non-limiting embodiments of endogenous Lamassu systems and the respective location of their components are provided in Table 16 herein.

In some embodiments, the components of a Lamassu system may be identified by the presence of similar domains present within each component. In some embodiments, domains comprise pfam domains and or clusters of orthologous groups (COG) domains.

In some embodiments, the term “LmuA” refers to the polynucleotide or expression product e.g., the polypeptide encoded by the lmuA gene. In some embodiments, the term “LmuA” refers to a LmuA polypeptide. In some embodiments, the lmuA gene encodes a polypeptide comprising a pfam14130 domain. In some embodiments, the lmuA gene encodes a polypeptide comprising a DUF4297 domain. In some embodiments, the lmuA gene encodes a polypeptide comprising a pfam14130 domain and a DUF4297 domain. In some embodiments, the LmuA polypeptide comprises a pfam14130 domain. In some embodiments, the LmuA polypeptide comprises a DUF4297 domain. In some embodiments, the LmuA polypeptide comprises a pfam14130 domain and a DUF4297 domain.

In some embodiments, LmuA polypeptide is encoded by a gene positioned within 5-60 genes of a gene encoding a LmuB polypeptide in a genome of a prokaryotic cell. In some embodiments, LmuA polypeptide is encoded by a gene positioned within 5 genes upstream (5′) or downstream (3′) to a gene encoding a LmuB polypeptide in a genome of a prokaryotic cell. In some embodiments, LmuA polypeptide is encoded by a gene positioned within 5 genes upstream (5′) and downstream (3′) to a gene encoding a LmuB polypeptide in a genome of a prokaryotic cell.

In some embodiments, LmuA and LmuB are encoded by genes positioned sequentially 5′ to 3′ in a genome of a prokaryotic cell. In some embodiments, LmuA and LmuB are encoded by genes positioned contiguously 5′ to 3′ in a genome of a prokaryotic cell.

In some embodiments, lmuA and lmuB genes are positioned sequentially 5′ to 3′ in a genome of a prokaryotic cell. In some embodiments, lmuA and lmuB, genes are positioned contiguously 5′ to 3′ in a genome of a prokaryotic cell.

As used herein, the term “LmuA” refers to the polynucleotide or expression product e.g., polypeptide encoded by the lmuA gene. In some embodiments, the term “LmuA” refers to a LmuA polypeptide. In some embodiments, the product of the lmuA gene comprises a pfam14130 domain. In some embodiments, the lmuA gene encodes a DUF4297 domain. In some embodiments, the product of the lmuA gene comprises a pfam14130 domain or a DUF4297 domain, or any combination thereof. In some embodiments, the product of the lmuA gene comprises a pfam14130 domain and a DUF4297 domain.

In some embodiments, LmuA polypeptide is encoded by a gene positioned within 5-60 genes of a gene encoding a LmuB polypeptide in a genome of a prokaryotic cell. In some embodiments, LmuA polypeptide is encoded by a gene positioned within 5 genes upstream (5′) or downstream (3′) to a gene encoding a LmuB polypeptide in a genome of a prokaryotic cell. In some embodiments, LmuA polypeptide is encoded by a gene positioned within 5 genes upstream (5′) and downstream (3′) to a gene encoding a LmuB polypeptide in a genome of a prokaryotic cell.

In some embodiments, a LmuA polypeptide is about 361 amino acids long (median gene size).

In some embodiments, the LmuA polypeptide comprises the amino acid sequence having at least 80% homology to a sequence selected from the group consisting of the polypeptides referenced in Table 16, rows 2-698, columns H and I. In some embodiments, the LmuA polypeptide comprises the amino acid sequence having at least 80% homology within similar domain regions within a sequence selected from the group consisting of the polypeptides referenced in Table 16, rows 2-698, columns H and I. In some embodiments, the LmuA polypeptide comprises a homolog of a polypeptide having the amino acid sequences set forth in the polypeptides referenced in Table 16, rows 2-698, columns H and I.

In some embodiments, the LmuA polypeptide comprises the amino acid sequence having at least 80% identity to a sequence selected from the group consisting of the polypeptides referenced in Table 16, rows 2-698, columns H and I. In some embodiments, the LmuA polypeptide comprises the amino acid sequence having at least 80% identity within similar domain regions within a sequence selected from the group consisting of the polypeptides referenced in Table 16, rows 2-698, columns H and I.

In some embodiments, the LmuA polypeptide comprises an amino acid sequence having at least 80%, at least 85%, at least 90%, or at least 95% homology to a sequence selected from the group consisting of the polypeptides referenced in Table 16, rows 2-698, columns H and I. In some embodiments, the LmuA polypeptide comprises the amino acid sequence having at least 80%, 85%, 90%, 95% homology within similar domain regions within a sequence selected from the group consisting of the polypeptides referenced in Table 16, rows 2-698, columns H and I. In some embodiments, the LmuA polypeptide comprises an amino acid sequence having at least 80%, at least 85%, at least 90%, or at least 95% identity to a sequence selected from the group consisting of the polypeptides referenced in Table 16, rows 2-698, columns H and I. In some embodiments, the LmuA polypeptide comprises the amino acid sequence having at least 80%, 85%, 90%, 95% identity within similar domain regions within a sequence selected from the group consisting of the polypeptides referenced in Table 16, rows 2-698, columns H and I. In some embodiments, the LmuA polypeptide comprises an amino acid sequence selected from the group consisting of the polypeptides referenced in Table 16, rows 2-698, columns H and I.

In some embodiments, the LmuA polypeptide comprises an amino acid sequence encoded by a nucleic acid sequence having at least 80%, at least 85%, at least 90%, or at least 95% homology to a sequence selected from the group consisting of the polynucleotides referenced in Table 16, rows 2-698, columns H and J. In some embodiments, the LmuA polypeptide comprises the amino acid sequence encoded by a nucleic acid sequence having at least 80%, 85%, 90%, 95% homology within similar domain regions within a sequence selected from the group consisting of the polynucleotides referenced in Table 16, rows 2-698, columns H and J. In some embodiments, the LmuA polypeptide comprises an amino acid sequence encoded by a nucleic acid sequence having at least 80%, at least 85%, at least 90%, or at least 95% identity to a sequence selected from the group consisting of the polynucleotides referenced in Table 16, rows 2-698, columns H and J. In some embodiments, the LmuA polypeptide comprises the amino acid sequence encoded by a nucleic acid sequence having at least 80%, 85%, 90%, 95% homology within similar domain regions within a sequence selected from the group consisting of the polynucleotides referenced in Table 16, rows 2-698, columns H and J. In some embodiments, the LmuA polypeptide comprises an amino acid sequence encoded from a nucleic acid sequence selected from the group consisting of the polynucleotides referenced in Table 16, rows 2-698, columns H and J.

In some embodiments, the nucleic acid sequence of a lmuA gene comprises a nucleic acid sequence having at least 80%, at least 85%, at least 90%, or at least 95% homology to a sequence selected from the group consisting of the polynucleotides referenced in Table 16, rows 2-698, columns H and J. In some embodiments, the lmuA gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 16, rows 2-698, columns H and J. In some embodiments, the nucleic acid sequence of a lmuA gene comprises a nucleic acid sequence having at least 80%, at least 85%, at least 90%, or at least 95% identity to a sequence selected from the group consisting of the polynucleotides referenced in Table 16, rows 2-698, columns H and J. In some embodiments, the lmuA gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 16, rows 2-698, columns H and J. In some embodiments, the nucleic acid sequence of a lmuA gene comprises a nucleic acid sequence selected from the group consisting of the polynucleotides referenced in Table 16, rows 2-698, columns H and J.

As used herein, the term “LmuB” refers to the polynucleotide or expression product e.g., polypeptide encoded by the lmuB gene. In some embodiments, the term “LmuB” refers to a LmuB polypeptide. In some embodiments, the product of the lmuB gene comprises a pfam02463 domain. In some embodiments, the lmuB gene encodes a RecF/RecN/SMC N-terminal domain.

In some embodiments, LmuB polypeptide is encoded by a gene positioned within 5-60 genes of a gene encoding a LmuA polypeptide in a genome of a prokaryotic cell. In some embodiments, LmuB polypeptide is encoded by a gene positioned within 5 genes upstream (5′) or downstream (3′) to a gene encoding a LmuA polypeptide in a genome of a prokaryotic cell. In some embodiments, LmuB polypeptide is encoded by a gene positioned within 5 genes upstream (5′) and downstream (3′) to a gene encoding a LmuA polypeptide in a genome of a prokaryotic cell.

In some embodiments, a LmuB polypeptide is about 559 amino acids long (median gene size).

In some embodiments, the LmuB polypeptide comprises an amino acid sequence having at least 80% homology to a sequence selected from the group consisting of the polypeptides referenced in Table 16, rows 2-698, columns L and M. In some embodiments, the LmuB polypeptide comprises the amino acid sequence having at least 80% homology within similar domain regions within a sequence selected from the group consisting of the polypeptides referenced in Table 16, rows 2-698, columns L and M. In some embodiments, a homologue of the LmuB polypeptide comprises a RecF/RecN/SMC N-terminal domain. In some embodiments, the LmuB polypeptide comprises an amino acid sequence having at least 80% identity to a sequence selected from the group consisting of the polypeptides referenced in Table 16, rows 2-698, columns L and M.

In some embodiments, the LmuB polypeptide comprises the amino acid sequence having at least 80% identity to a sequence selected from the group consisting of the polypeptides referenced in Table 16, rows 2-698, columns L and M. In some embodiments, the LmuB polypeptide comprises the amino acid sequence having at least 80% identity within similar domain regions within a sequence selected from the group consisting of the polypeptides referenced in Table 16, rows 2-698, columns L and M.

In some embodiments, the LmuB polypeptide comprises an amino acid sequence having at least 80%, at least 85%, at least 90%, or at least 95% homology to a sequence selected from the group consisting of the polypeptides referenced in Table 16, rows 2-698, columns L and M. In some embodiments, the LmuB polypeptide comprises the amino acid sequence having at least 80%, 85%, 90%, 95% homology within similar domain regions within a sequence selected from the group consisting of the polypeptides referenced in Table 16, rows 2-698, columns L and M. In some embodiments, the LmuB polypeptide comprises an amino acid sequence having at least 80%, at least 85%, at least 90%, or at least 95% identity to a sequence selected from the group consisting of the polypeptides referenced in Table 16, rows 2-698, columns L and M. In some embodiments, the LmuB polypeptide comprises the amino acid sequence having at least 80%, 85%, 90%, 95% identity within similar domain regions within a sequence selected from the group consisting of the polypeptides referenced in Table 16, rows 2-698, columns L and M. In some embodiments, the LmuB polypeptide comprises an amino acid sequence selected from the group consisting of the polypeptides referenced in Table 16, rows 2-698, columns L and M.

In some embodiments, the LmuB polypeptide comprises an amino acid sequence encoded by a nucleic acid sequence having at least 80%, at least 85%, at least 90%, or at least 95% homology to a sequence selected from the group consisting of the polynucleotides referenced in Table 16, rows 2-698, columns L and N. In some embodiments, the LmuB polypeptide comprises the amino acid sequence encoded by a nucleic acid sequence having at least 80%, 85%, 90%, 95% homology within similar domain regions within a sequence selected from the group consisting of the polynucleotides referenced in Table 16, rows 2-698, columns L and N. In some embodiments, the LmuB polypeptide comprises an amino acid sequence encoded by a nucleic acid sequence having at least 80%, at least 85%, at least 90%, or at least 95% identity to a sequence selected from the group consisting of the polynucleotides referenced in Table 16, rows 2-698, columns L and N. In some embodiments, the LmuB polypeptide comprises the amino acid sequence encoded by a nucleic acid sequence having at least 80%, 85%, 90%, 95% homology within similar domain regions within a sequence selected from the group consisting of the polynucleotides referenced in Table 16, rows 2-698, columns L and N. In some embodiments, the LmuB polypeptide comprises an amino acid sequence encoded from a nucleic acid sequence selected from the group consisting of the polynucleotides referenced in Table 16, rows 2-698, columns L and N.

In some embodiments, the nucleic acid sequence of a lmuB gene comprises a nucleic acid sequence having at least 80%, at least 85%, at least 90%, or at least 95% homology to a sequence selected from the group consisting of the polynucleotides referenced in Table 16, rows 2-698, columns L and N. In some embodiments, the lmuB gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 16, rows 2-698, columns L and N. In some embodiments, the nucleic acid sequence of a lmuB gene comprises a nucleic acid sequence having at least 80%, at least 85%, at least 90%, or at least 95% identity to a sequence selected from the group consisting of the polynucleotides referenced in Table 16, rows 2-698, columns L and N. In some embodiments, the lmuB gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 16, rows 2-698, columns L and N. In some embodiments, the lmuB gene homolog encodes a polypeptide comprising a RecF/RecN/SMC N-terminal domain. In some embodiments, the nucleic acid sequence of a lmuB gene comprises a nucleic acid sequence selected from the group consisting of the polynucleotides referenced in Table 16, rows 2-698, columns L and N.

A skilled artisan would appreciate that the terms a “functional portion of a Defense System VIII component” or “functional fragment of Defense System VIII component” or “functional portion of a Lamassu defense system component” or “functional fragment of Lamassu defense system component” refers to a functional portion of a Lamassu polynucleotide or polypeptide, as disclosed herein, which expression is sufficient to elicit an anti-phage activity alone or in combination with the at least one of the other Lamassu polynucleotides or polypeptides disclosed herein or functional portions thereof.

The terms “LmuA”, “LmuB”, “lmuA” and “lmuB”, also refer to functional LmuA, LmuB, lmuA and lmuB, homologs, which exhibit the desired activity (i.e., conferring phage resistance). Such homologs can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide sequences referenced in Table 16 rows 2-698 columns H and I and L and M, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polynucleotide sequences referenced in Table 16 rows 2-698 columns H and J and L and N, respectively, respectively. In some embodiments, such homologs comprise at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% homology or identity within similar domain regions of the polypeptide sequences referenced in Table 16 rows 2-698 columns H and I and L and M, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or homology within nucleotide sequences encoding similar domain regions to the polynucleotide sequences referenced in Table 16 rows 2-698 columns H and J and L and N, respectively.

Table 16 presents embodiments of components of Defense System VIII that may be found in a diverse array of bacteria and archaea genomes (referenced in Table 18). Table 16 presents embodiments of nucleic acid sequences encoding gene cassettes of Defense System VIII (referenced in Table 18).

The Kiwa Defense System

In some embodiments, a defense system disclosed herein comprises a Kiwa anti-phage defense system. (Table 17)

In some embodiments, a Kiwa defense system provides a host cell with resistance to entry of foreign nucleic acid invasion. In some embodiments, a host cell expressing a functional Kiwa defense system (Defense System IX) provides the host cell resistance foreign nucleic acid invasion.

In some embodiments, a Kiwa defense system provides a host cell with resistance to at least one phage.

In some embodiments, a microbial cell comprising a Kiwa defense system protects the microbial cell from phage infection. In some embodiments, a microbial cell expressing a Kiwa defense system protects the microbial cell from phage infection. In some embodiments, a microbial cell expressing a functional Kiwa defense system protects the microbial cell from phage infection. In some embodiments, a bacterial cell comprising a Kiwa defense system protects the bacterial cell from phage infection. In some embodiments, a bacterial cell expressing a Kiwa defense system protects the bacterial cell from phage infection. In some embodiments, a bacterial cell expressing a functional Kiwa defense system protects the bacterial cell from phage infection.

In some embodiments, a Kiwa defense system provides a host cell with resistance to a plasmid.

As used herein, the term “a Kiwa anti-phage defense system” may be used interchangeably with the term “a Defense System IX”, having all the same meanings and qualities. A skilled artisan would appreciate that the term “Kiwa system” may be used interchangeably in some embodiments with “Kiwa defense system”, and “Kiwa the defense system”, “Kiwa anti-phage system”, having all the same meanings and qualities.

In some embodiments, a microbial species does not comprise an endogenous Defense System IX. In some embodiments, a microbial species does not express an endogenous Defense System IX. In some embodiments, a microbial species does not express an endogenous functional Defense System IX.

In some embodiments, a bacterial species does not comprise an endogenous Defense System IX. In some embodiments, a bacterial species does not express an endogenous Defense System IX. In some embodiments, a bacterial species does not express an endogenous functional Defense System IX.

A Kiwa defense system comprises, in some embodiments, a nucleic acid construct comprising a nucleic acid sequence encoding a KwaA polypeptide and a KwaB polypeptide comprising a pfam16162 domain or a DUF4868 domain or a combination thereof. In some embodiments, a Kiwa defense system comprises, a nucleic acid construct comprising a nucleic acid sequence encoding a KwaA polypeptide and a KwaB polypeptide comprising a pfam16162 domain or a DUF4868 domain or a combination thereof, wherein said KwaA polypeptide is encoded by a gene positioned within 5 genes of a gene encoding a KwaB polypeptide in the genome of a prokaryotic cell. In some embodiments, a Kiwa defense system comprises a nucleic acid construct comprising a nucleic acid sequence comprising a kwaA gene and a kwaB gene.

In some embodiments, a Kiwa defense system comprises, a KwaA polypeptide and a KwaB polypeptide comprising a pfam16162 domain or a DUF4868 domain or a combination thereof. In some embodiments, a Kiwa defense system comprises, a KwaA polypeptide and a KwaB polypeptide comprising a pfam16162 domain or a DUF4868 domain or a combination thereof, wherein said KwaA polypeptide is encoded by a gene positioned within 5 genes of a gene encoding a KwaB polypeptide in the genome of a prokaryotic cell. In some embodiments, a Kiwa defense system comprises KwaA polypeptide and a KwaB polypeptide.

A Kiwa defense system comprises, in some embodiments, a nucleic acid construct comprising a nucleic acid sequence encoding a KwaA polypeptide and a KwaB polypeptide comprising a pfam16162 domain or DUF4868 domain or a combination thereof. In some embodiments, a Kiwa defense system comprises a nucleic acid construct comprising a nucleic acid sequence encoding a KwaA polypeptide and a KwaB polypeptide.

In some embodiments, a Defense System IX comprises a KwaA polypeptide comprising an amino acid sequence having at least 80% identity to a sequence selected from the group referenced in Table 17 rows 2-935 columns H and I; or a KwaB polypeptide comprising a pfam16162 domain or a DUF4868 domain or a combination thereof, or comprising an amino acid sequence having at least 80% identity to a sequence selected from the group referenced in Table 17 rows 2-935 columns H and I; or a combination thereof.

In some embodiments, a Defense System IX comprises at least two different polypeptide components selected from a KwaA polypeptide comprising an amino acid sequence having at least 80% identity to a sequence selected from the group referenced in Table 17 rows 2-935 columns H and I; and a KwaB polypeptide comprising a pfam16162 domain or a DUF4868 domain or a combination thereof, or comprising an amino acid sequence having at least 80% identity to a sequence selected from the group referenced in Table 17 rows 2-935 columns H and I.

In some embodiments, a Kiwa defense system having an anti-phage activity comprises a nucleic acid construct comprising a nucleic acid sequence encoding a KwaA polypeptide and a KwaB polypeptide. In some embodiments, a Kiwa defense system having an anti-phage activity comprises a nucleic acid construct comprising a nucleic acid sequence comprising a kwaA gene and a kwaB gene.

In some embodiments, the Kiwa anti-phage defense system comprises a nucleic acid construct comprising nucleic acid sequences encoding a cassette comprising kwaA and kwaB genes.

In some embodiments, a Kiwa (Defense System IX) gene cassette comprises the nucleic acid sequence:

(SEQ ID NO: 3; construct 2 in Table 4)

| | |
|---|---|
| 1 | taatggtctt ccttccattg gtatctcgtg agtatacaag caatatcgaa tcggcaaata |
| 61 | tactcattag tatactcaca agcctgttga tgtgggttgg ttcaggttga cttcagttga |
| 121 | ggagaaaagc tggaaaagcc ttgcgtggcg cggatttcag gcataaaaaa agacctcagt |
| 181 | tgaggtctat ttacatactt ttggtgcgaa ggccggactc gaacatcgca actatcctta |
| 241 | tgatcttaat ggattaaaaa ttaccatatt ggtcatgata ccatcattga taccatcaga |
| 301 | cccgattgat gataatcgaa cagtaataaa atatgactaa acaatttatg cttcaacata |
| 361 | agattgaatg ctttgatcta ccattttaat gcagtggaaa aatttttaac acaatactta |
| 421 | agaaaaataa tttcatataa aaactacatt tcatttcata tagttaacac accaaaaaca |
| 481 | actgtatata aacccagtaa aaaatggtag aatcactatt gtgttcaatt gttgaacatt |
| 541 | tataatacat ttgtcggata ggggggtgtt tatgcaaagg aacacataca acaaagtagg |
| 601 | tctatacatc ctatctttag ctatgctttt tgtttttatt attatcctga ctgccaaaat |
| 661 | ccccttttgc ttcacttcgg attgtagctt cataggtttg aagaaacttg tattaacaaa |
| 721 | tattgttcct attgtttgct ttgtattttt tctatttagt atttattttt ataatagatt |
| 781 | aaaaaacata acaaaataca atggacaaga tagcgttaaa ataacatcat gtcaaagcga |
| 841 | aagctatgaa agtttaacat tcttagcaac atacattgta ccttttatgg ggtttagttt |
| 901 | tgaagacatg caaaagaata tagcatatct tttgcttgta gtagtaatag gcattatttt |
| 961 | tattaaaaca gacaagtatt atgcgaatcc aactttagct ctgtttggct ttaaactata |
| 1021 | cagggtaaat atactacacc caggaagtgg agaaacaaaa aatctgatag ctatttcaaa |
| 1081 | tgatgtatta aaagtggacg ataatgttta ttatagtttc tttgacgaat ttgtttttat |
| 1141 | agcaaggaaa aaaatatgac tactcagcag ttaaaagaaa aaatttcaaa aataattgat |
| 1201 | aactttagcg gaatcagggt tgtatttact actactgcta atgaactaaa gctttccaga |
| 1261 | attgaaggga gtgcattaaa tagcatagct gaagggttta ttgacaaaat caaagaagac |
| 1321 | attataaata atgaagatct cacctcaccc ttgctgtcca attttgatga tagaaaaaat |
| 1381 | gccttgttta aatttgatta cgaacaatat cctgaagaat ttaacaaaat cactcaggcc |
| 1441 | atcgctatcc ctcctaactc acaagattat tataaccctt taaataaatt cacagatgtg |
| 1501 | aaaggaatta taattttaat aagtggagat aacaaatgcc tggctcttta taaaaacaaa |
| 1561 | actaaccttg cagtattgag aaatagcaga aaaatgttta atttagtccc tgaccctgat |
| 1621 | ggatacttga aacaactccc taatgagata ttacggttag atttcaatta tgatctattt |
| 1681 | tcaattggtg aggattttta cattaaaaat cacaagactc ttgaaactca aatgaaattt |
| 1741 | catcaagtga ttgaagccca agcagttatt gctttaaatt ctttacggga ttcactacta |
| 1801 | attgaagaca tatcaggttt agaaaagagt tcgagagaaa tttcattcgc tagaaagttg |
| 1861 | gcaaaaatct ccaaacattc tcctgttttg gggaaaatag atactaaaac aattattgat |
| 1921 | tatgtatccc aacataaata tttatctgca atacttcaga ttaatgaagc gggagataag |
| 1981 | ttattaatta aaaccaagac atcacagaag catttcatta aactcatgag cgatgattat |
| 2041 | ttacaatcag atctaaccaa aataatatac atgagcatag caaaagacag attagatgaa |
| 2101 | tagaacttta agtagaagtg acaaatatta taaaccattc ttgaaatata tttgtcacta |
| 2161 | ttactaat. |

The coding regions for each of the kwaA and kwaB gene sequences within this embodiment of a Kiwa cassette (SEQ ID NO: 3) are as follows: nucleotides 572-1159 encode an embodiment of a KwaA polypeptide, and nucleotides 1156-2103 encode an embodiment of a KwaB polypeptide.

In some embodiments, a Defense System IX comprising a KwaA polypeptide and a KwaB polypeptide is encoded by the nucleic acid sequence set forth in SEQ ID NO: 3. In some embodiments, a Defense System IX comprising a KwaA polypeptide and a KwaB polypeptide, is encoded by a nucleic acid sequence having at least 80% homology to the sequence set forth in SEQ ID NO: 3. In some embodiments, a Defense System IX comprising a KwaA polypeptide and a KwaB polypeptide, is encoded by a nucleic acid sequence having at least 85% homology, or 90% homology, or 95% homology, or 97% homology, or 98% homology, or 99% homology to the sequence set forth in SEQ ID NO: 3. In some embodiments, a Defense System IX comprising a KwaA polypeptide and a KwaB polypeptide, is encoded by a nucleic acid sequence having at least 80% identity to the sequence set forth in SEQ ID NO: 3. In some embodiments, a Defense System IX comprising a KwaA polypeptide and a KwaB polypeptide, is encoded by a nucleic acid sequence having at least 85% identity, or 90% identity, or 95% identity, or 97% identity, or 98% identity, or 99% identity to the sequence set forth in SEQ ID NO: 3.

In some embodiments, a construct comprising the Kiwa defense system encodes one component of the defense system, whereby multiple constructs may be used to assemble the functional defense system. In some embodiments, the components of a Kiwa defense system comprise genes kwaA and kwaB. In some embodiments, the components of a Kiwa defense system consist of genes kwaA and kwaB. In some embodiments, the components of a Kiwa defense system comprise nucleic acid sequences encoding a KwaA polypeptide and a KwaB polypeptide. In some embodiments, the components of a Kiwa defense system consist of nucleic acid sequences encoding a KwaA polypeptide and a KwaB polypeptide.

In some embodiments, a construct comprising the Kiwa defense system encodes one component of the defense system, whereby a second construct may be used to assemble the functional defense system. In some embodiments, a construct comprising the Kiwa defense system encodes one component of the defense system, whereby additional constructs may be used to assemble the functional defense system. For example, in some embodiments each of KwaA and KwaB may be encoded by nucleic acid sequences comprised in different constructs, wherein expression of the combination of constructs produces a functional Kiwa defense system.

In some embodiments, the components making up a functional Kiwa anti-phage defense system comprise a KwaA polypeptide and a KwaB polypeptide, each encoded by a kwaA and a kwaB gene, respectively.

In some embodiments, a Kiwa defense system having an anti-phage activity comprise a nucleic acid encoding a KwaA polypeptide. In some embodiments, a Kiwa defense system having an anti-phage activity comprise a nucleic acid encoding a KwaA polypeptide and a KwaB polypeptide.

In some embodiments, a Kiwa defense system having an anti-phage activity comprise a nucleic acid comprising a kwaA gene. In some embodiments, a Kiwa defense system having an anti-phage activity comprise a nucleic acid comprising a kwaA gene and a kwaB gene.

In some embodiments, a Kiwa defense system having an anti-phage activity comprises a nucleic acid construct comprising nucleic acids encoding polypeptides in a set order. In one embodiment, the 5′ to 3′ order of polypeptides encoded is KwaA and KwaB. In some embodiments the 5′ to 3′ order of polypeptides does not affect the anti-phage activity. In some embodiments the 5′ to 3′ order of polypeptides does affect the anti-phage activity.

In some embodiments, the 5′ to 3′ order of polypeptides is random, for example any order of KwaA and KwaB.

In some embodiments, a Kiwa defense system having an anti-phage activity comprises a nucleic acid construct comprising genes in a set order. In some embodiment, the 5′ to 3′ order of genes is kwaA and kwaB. In some embodiment, the 5′ to 3′ order of genes in a Kiwa defense system is not kwaA and kwaB. In some embodiments the 5′ to 3′ order of genes does not affect the anti-phage activity. In some embodiments the 5′ to 3′ order of genes does affect the anti-phage activity.

In some embodiments, the 5′ to 3′ order of genes is random, for example any order of kwaA and kwaB.

In some embodiments, the Kiwa system (Defense System IX) composition and order is as shown in FIG. 3B.

In some embodiments, a Kiwa defense system having an anti-phage activity originates from a microbial genome, for example a bacterial or an archaeal genome (Table 17). A skilled artisan would appreciate that the Kiwa system is not present in the majority of bacteria and or archaea species.

In some embodiments, a functional Kiwa defense system comprises a construct comprising nucleic acid sequences encoding polypeptides, wherein the nucleic acid sequence encoding each polypeptide may originate from a different microbial species. For example, the source of the nucleic acid sequence encoding a KwaA polypeptide may be one microbial species, while the source of the nucleic acid sequence encoding a KwaB polypeptide may be a different microbial species. In some embodiments, a functional Kiwa defense system comprises a non-naturally occurring combination of polypeptide components. In some embodiments, a functional Kiwa defense system comprises a combination of at least two polypeptides that do not naturally occur together.

In some embodiments, a functional Kiwa defense system comprises a construct comprising nucleic acid sequences encoding polypeptides, wherein the nucleic acid sequence encoding each polypeptide may originate from a different bacterial species. For example, the source of the nucleic acid sequence encoding a KwaA polypeptide may be one bacterial species, while the source of the nucleic acid sequence encoding a KwaB polypeptide may be a different bacterial species.

In some embodiments, the source of the nucleic acid encoding a KwaA polypeptide and a KwaB polypeptide is the same. In some embodiments, the source of the nucleic acid encoding a KwaA polypeptide and a KwaB polypeptide is the not the same. In some embodiments, the source of the nucleic acid sequence of any of the components of a Kiwa defense system comprises any of the species listed in Table 17.

In some embodiments, a Kiwa defense system having an anti-phage activity comprises a nucleic acid construct comprising non-coding regions between the nucleic acid sequences encoding the polypeptides. In some embodiments, the non-coding regions between the nucleic acid sequences comprises nucleic acid sequence not naturally occurring in the microbial species of origin. In some embodiments, the non-coding regions between the nucleic acid sequences comprises nucleic acid sequence is the naturally occurring in the microbial species of origin.

In some embodiments, a Kiwa system disclosed herein comprises a multi-gene phage resistance system broadly distributed in microbial genomes, for example but not limited to bacteria and archaea. According to some embodiments, the Kiwa system components are located in a gene cluster (a cassette of genes) in a microbial cell genome. According to some embodiments, the Kiwa system components are located in close proximity (e.g. positioned within 20 genes, 10 genes, 5 genes or less one from the other) in a genome of a prokaryotic cell. According to some embodiments the prokaryotic cell expresses an endogenous Kiwa defense system. According to some embodiments, the prokaryotic cell expresses an endogenous functional Kiwa defense system. According to some embodiments, the species of prokaryotic cell is selected from the group consisting of the species listed in Table 17. According to some embodiments a prokaryotic cell expresses a non-endogenous Kiwa defense system. According to some embodiments, a prokaryotic cell expresses a non-endogenous functional Kiwa defense system. According to some embodiments, the species of prokaryotic cell expressing a non-endogenous functional Kiwa defense system is selected from the group consisting of the species listed in Table 17.

In some embodiments, Kiwa defense system components comprise KwaA and KwaB polypeptides. In some embodiments, a Kiwa defense system components comprise functional portions of KwaA and KwaB polypeptides. In some embodiments, the Kiwa defense system components are encoded by kwaA and kwaB genes.

Non-limiting embodiments of endogenous Kiwa systems and the respective location of their components are provided in Table 17 herein.

In some embodiments, the components of a Kiwa system may be identified by the presence of similar domains present within each component. In some embodiments, domains comprise pfam domains and or clusters of orthologous groups (COG) domains.

In some embodiments, a Defense System IX comprises a membrane associated complex.

In some embodiments, the term “KwaA” refers to the polynucleotide or expression product e.g., the polypeptide encoded by the kwaA gene. In some embodiments, the term “KwaA” refers to a KwaA polypeptide. In some embodiments, a KwaA polypeptide comprises transmembrane helices. In some embodiments, a kwaA gene encodes a polypeptide comprising transmembrane helices.

In some embodiments, KwaA polypeptide is encoded by a gene positioned within 5-60 genes of a gene encoding a KwaB polypeptide in a genome of a prokaryotic cell. In some embodiments, KwaA polypeptide is encoded by a gene positioned within 5 genes upstream (5′) or downstream (3′) to a gene encoding a KwaB polypeptide in a genome of a prokaryotic cell. In some embodiments, KwaA polypeptide is encoded by a gene positioned within 5 genes upstream (5′) and downstream (3′) to a gene encoding a KwaB polypeptide in a genome of a prokaryotic cell.

In some embodiments, KwaA and KwaB are encoded by genes positioned sequentially 5′ to 3′ in a genome of a prokaryotic cell. In some embodiments, KwaA and KwaB are encoded by genes positioned contiguously 5′ to 3′ in a genome of a prokaryotic cell. In some embodiments, nucleic acid encoding additional copies of KwaB is positions 3′ of the nucleic acid sequence first encoding a KwaB polypeptide.

In some embodiments, kwaA and kwaB genes are positioned sequentially 5′ to 3′ in a genome of a prokaryotic cell. In some embodiments, kwaA and kwaB, genes are positioned contiguously 5′ to 3′ in a genome of a prokaryotic cell.

In some embodiments, a KwaA polypeptide is about 194 amino acids long (median gene size).

In some embodiments, the KwaA polypeptide comprises the amino acid sequence having at least 80% homology to a sequence selected from the group consisting of the polypeptides referenced in Table 17, rows 2-935, columns H and I. In some embodiments, the KwaA polypeptide comprises the amino acid sequence having at least 80% homology within similar domain regions to a sequence selected from the group consisting of the polypeptides referenced in Table 17, rows 2-935, columns H and I. In some embodiments, a KwaA homolog comprises transmembrane helices. In some embodiments, the KwaA polypeptide comprises a homolog of a polypeptide having the amino acid sequences set forth in the polypeptides referenced in Table 17, rows 2-935, columns H and I.

In some embodiments, the KwaA polypeptide comprises the amino acid sequence having at least 80% identity to a sequence selected from the group consisting of the polypeptides referenced in Table 17, rows 2-935, columns H and I. In some embodiments, the KwaA polypeptide comprises the amino acid sequence having at least 80% identity within similar domain regions to a sequence selected from the group consisting of the polypeptides referenced in Table 17, rows 2-935, columns H and I.

In some embodiments, the KwaA polypeptide comprises the amino acid sequence having at least 80% identity to a sequence selected from the group consisting of the polypeptides referenced in Table 17, rows 2-935, columns H and I. In some embodiments, the KwaA polypeptide comprises the amino acid sequence having at least 80% identity within similar domain regions within a sequence selected from the group consisting of the polypeptides referenced in Table 17, rows 2-935, columns H and I.

In some embodiments, the KwaA polypeptide comprises an amino acid sequence having at least 80%, at least 85%, at least 90%, or at least 95% homology to a sequence selected from the group consisting of the polypeptides referenced in Table 17, rows 2-935, columns H and I. In some embodiments, the KwaA polypeptide comprises the amino acid sequence having at least 80%, 85%, 90%, 95% homology within similar domain regions within a sequence selected from the group consisting of the polypeptides referenced in Table 17, rows 2-935, columns H and I. In some embodiments, the KwaA polypeptide comprises an amino acid sequence having at least 80%, at least 85%, at least 90%, or at least 95% identity to a sequence selected from the group consisting of the polypeptides referenced in Table 17, rows 2-935, columns H and I. In some embodiments, the KwaA polypeptide comprises the amino acid sequence having at least 80%, 85%, 90%, 95% identity within similar domain regions within a sequence selected from the group consisting of the polypeptides referenced in Table 17, rows 2-935, columns H and I. In some embodiments, the KwaA polypeptide comprises an amino acid sequence selected from the group consisting of the polypeptides referenced in Table 17, rows 2-935, columns H and I.

In some embodiments, the KwaA polypeptide comprises an amino acid sequence encoded by a nucleic acid sequence having at least 80%, at least 85%, at least 90%, or at least 95% homology to a sequence selected from the group consisting of the polynucleotides referenced in Table 17, rows 2-935, columns H and J. In some embodiments, the KwaA polypeptide comprises the amino acid sequence encoded by a nucleic acid sequence having at least 80%, 85%, 90%, 95% homology within similar domain regions within a sequence selected from the group consisting of the polynucleotides referenced in Table 17, rows 2-935, columns H and J. In some embodiments, the KwaA polypeptide comprises an amino acid sequence encoded by a nucleic acid sequence having at least 80%, at least 85%, at least 90%, or at least 95% identity to a sequence selected from the group consisting of the polynucleotides referenced in Table 17, rows 2-935, columns H and J. In some embodiments, the KwaA polypeptide comprises the amino acid sequence encoded by a nucleic acid sequence having at least 80%, 85%, 90%, 95% homology within similar domain regions within a sequence selected from the group consisting of the polynucleotides referenced in Table 17, rows 2-935, columns H and J. In some embodiments, the KwaA polypeptide comprises an amino acid sequence encoded from a nucleic acid sequence selected from the group consisting of the polynucleotides referenced in Table 17, rows 2-935, columns H and J.

In some embodiments, the nucleic acid sequence of a kwaA gene comprises a nucleic acid sequence having at least 80%, at least 85%, at least 90%, or at least 95% homology to a sequence selected from the group consisting of the polynucleotides referenced in Table 17, rows 2-935, columns H and J. In some embodiments, the kwaA gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 17, rows 2-935, columns H and J. In some embodiments, the nucleic acid sequence of a kwaA gene comprises a nucleic acid sequence having at least 80%, at least 85%, at least 90%, or at least 95% identity to a sequence selected from the group consisting of the polynucleotides referenced in Table 17, rows 2-935, columns H and J. In some embodiments, the kwaA gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 17, rows 2-935, columns H and J. In some embodiments, a kwaA gene homolog encodes a polypeptide comprising transmembrane helices. In some embodiments, the nucleic acid sequence of a kwaA gene comprises a nucleic acid sequence selected from the group consisting of the polynucleotides referenced in Table 17, rows 2-935, columns H and J.

As used herein, the term “KwaB” refers to the polynucleotide or expression product e.g., polypeptide encoded by the kwaB gene. In some embodiments, the term “KwaB” refers to a KwaB polypeptide. In some embodiments, the product of the kwaB gene comprises a pfam16162 domain. In some embodiments, the product of the kwaB gene comprises a DUF4868 domain.

In some embodiments, KwaB polypeptide is encoded by a gene positioned within 5-60 genes of a gene encoding a KwaA polypeptide in a genome of a prokaryotic cell. In some embodiments, KwaB polypeptide is encoded by a gene positioned within 5 genes upstream (5′) or downstream (3′) to a gene encoding a KwaA polypeptide in a genome of a prokaryotic cell. In some embodiments, KwaB polypeptide is encoded by a gene positioned within 5 genes upstream (5′) and downstream (3′) to a gene encoding a KwaA polypeptide in a genome of a prokaryotic cell.

In some embodiments, KwaA and KwaB are encoded by genes positioned sequentially 5′ to 3′ in a genome of a prokaryotic cell. In some embodiments KwaA and KwaB are encoded by genes positioned contiguously 5′ to 3′ in a genome of a prokaryotic cell.

In some embodiments, a KwaB polypeptide is about 315 amino acids long (median gene size).

In some embodiments, the KwaB polypeptide comprises the amino acid sequence having at least 80% homology to a sequence selected from the group consisting of the polypeptides referenced in Table 17, rows 2-935, columns L and M. In some embodiments, the KwaB polypeptide comprises the amino acid sequence having at least 80% homology within similar domain regions within a sequence selected from the group consisting of the polypeptides referenced in Table 17, rows 2-935, columns L and M. In some embodiments, the KwaB polypeptide comprises a homolog of a polypeptide having the amino acid sequences set forth in the polypeptides referenced in Table 17, rows 2-935, columns L and M.

In some embodiments, the KwaB polypeptide comprises the amino acid sequence having at least 80% identity to a sequence selected from the group consisting of the polypeptides referenced in Table 17, rows 2-935, columns L and M. In some embodiments, the KwaB polypeptide comprises the amino acid sequence having at least 80% identity within similar domain regions within a sequence selected from the group consisting of the polypeptides referenced in Table 17, rows 2-935, columns L and M.

In some embodiments, the KwaB polypeptide comprises an amino acid sequence having at least 80%, at least 85%, at least 90%, or at least 95% homology to a sequence selected from the group consisting of the polypeptides referenced in Table 17, rows 2-935, columns L and M. In some embodiments, the KwaB polypeptide comprises the amino acid sequence having at least 80%, 85%, 90%, 95% homology within similar domain regions within a sequence selected from the group consisting of the polypeptides referenced in Table 17, rows 2-935, columns L and M. In some embodiments, the KwaB polypeptide comprises an amino acid sequence having at least 80%, at least 85%, at least 90%, or at least 95% identity to a sequence selected from the group consisting of the polypeptides referenced in Table 17, rows 2-935, columns L and M. In some embodiments, the KwaB polypeptide comprises the amino acid sequence having at least 80%, 85%, 90%, 95% identity within similar domain regions within a sequence selected from the group consisting of the polypeptides referenced in Table 17, rows 2-935, columns L and M. In some embodiments, the KwaB polypeptide comprises an amino acid sequence selected from the group consisting of the polypeptides referenced in Table 17, rows 2-935, columns L and M.

In some embodiments, the KwaB polypeptide comprises an amino acid sequence encoded by a nucleic acid sequence having at least 80%, at least 85%, at least 90%, or at least 95% homology to a sequence selected from the group consisting of the polynucleotides referenced in Table 17, rows 2-935, columns L and N. In some embodiments, the KwaB polypeptide comprises the amino acid sequence encoded by a nucleic acid sequence having at least 80%, 85%, 90%, 95% homology within similar domain regions within a sequence selected from the group consisting of the polynucleotides referenced in Table 17, rows 2-935, columns L and N. In some embodiments, the KwaB polypeptide comprises an amino acid sequence encoded by a nucleic acid sequence having at least 80%, at least 85%, at least 90%, or at least 95% identity to a sequence selected from the group consisting of the polynucleotides referenced in Table 17, rows 2-935, columns L and N. In some embodiments, the KwaB polypeptide comprises the amino acid sequence encoded by a nucleic acid sequence having at least 80%, 85%, 90%, 95% homology within similar domain regions within a sequence selected from the group consisting of the polynucleotides referenced in Table 17, rows 2-935, columns L and N. In some embodiments, the KwaB polypeptide comprises an amino acid sequence encoded from a nucleic acid sequence selected from the group consisting of the polynucleotides referenced in Table 17, rows 2-935, columns L and N.

In some embodiments, the nucleic acid sequence of a kwaB gene comprises a nucleic acid sequence having at least 80%, at least 85%, at least 90%, or at least 95% homology to a sequence selected from the group consisting of the polynucleotides referenced in Table 17, rows 2-935, columns L and N. In some embodiments, the kwaB gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 17, rows 2-935, columns L and N. In some embodiments, the nucleic acid sequence of a kwaB gene comprises a nucleic acid sequence having at least 80%, at least 85%, at least 90%, or at least 95% identity to a sequence selected from the group consisting of the polynucleotides referenced in Table 17, rows 2-935, columns L and N. In some embodiments, the kwaB gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 17, rows 2-935, columns L and N. In some embodiments, the nucleic acid sequence of a kwaB gene comprises a nucleic acid sequence selected from the group consisting of the polynucleotides referenced in Table 17, rows 2-935, columns L and N.

A skilled artisan would appreciate that the terms a “functional portion of a Defense System IX component” or “functional fragment of Defense System IX component” or “functional portion of a Kiwa defense system component” or “functional fragment of Kiwa defense system component” refers to a functional portion of a Kiwa polynucleotide or polypeptide, as disclosed herein, which expression is sufficient to elicit an anti-phage activity alone or in combination with the at least one of the other Kiwa polynucleotides or polypeptides disclosed herein or functional portions thereof.

The terms “KwaA”, “KwaB”, “kwaA” and “kwaB”, also refer to functional KwaA, KwaB, kwaA and kwaB homologs, which exhibit the desired activity (i.e., conferring phage resistance). Such homologs can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide sequences referenced in Table 17 rows 2-935 columns H and I and L and M, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polynucleotide sequences referenced in Table 17 rows 2-935 columns H and J and L and N, respectively, respectively. In some embodiments, such homologs comprise at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% homology or identity within similar domain regions of the polypeptide sequences referenced in Table 17 rows 2-935 columns H and I and L and M, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or homology within nucleotide sequences encoding similar domain regions to the polynucleotide sequences referenced in Table 17 rows 2-935 columns H and J and L and N, respectively.

Table 17 presents embodiments of components of Defense System IX that may be found in a diverse array of bacteria and archaea genomes (referenced in Table 18). Table 17 presents embodiments of nucleic acid sequences encoding gene cassettes of Defense System IX (referenced in Table 18).

The Wadjet Defense System

In some embodiments, a defense system disclosed herein comprises a Wadjet anti-plasmid defense system. (Table 11)

In some embodiments, a Wadjet defense system provides a host cell with resistance to entry of foreign nucleic acid invasion. In some embodiments, a host cell expressing a functional Wadjet defense system (Defense System Xa; Defense System Xb; or Defense System Xc) provides the host cell resistance foreign nucleic acid invasion.

In some embodiments, a Wadjet defense system provides a host cell with reduced efficiency of transformation of plasmids. In some embodiments, a Wadjet defense system provides a host cell with protective activity against plasmids.

In some embodiments, a Type I Wadjet defense system provides a host cell with reduced efficiency of transformation of plasmids. In some embodiments, a Wadjet Type I defense system provides a host cell with protective activity against plasmids. In some embodiments, a Type II Wadjet defense system provides a host cell with reduced efficiency of transformation of plasmids. In some embodiments, a Wadjet Type II defense system provides a host cell with protective activity against plasmids. In some embodiments, a Type III Wadjet defense system provides a host cell with reduced efficiency of transformation of plasmids. In some embodiments, a Wadjet Type III defense system provides a host cell with protective activity against plasmids.

In some embodiments, expression of a Wadjet Type I defense system in microbial cells protects the microbial cell from plasmid entry, reducing or eliminating plasmid transformation. In some embodiments, expression of a Wadjet Type II defense system in a microbial cell protects the microbial cell from plasmid entry, reducing or eliminating plasmid transformation. In some embodiments, expression of a Wadjet Type III defense system in a microbial cell protects the microbial cell from plasmid entry, reducing or eliminating plasmid transformation.

In some embodiments, expression of a Wadjet Type I defense system in bacterial cell protects the bacterial cell from plasmid entry, reducing or eliminating plasmid transformation. In some embodiments, expression of a Wadjet Type II defense system in bacteria protects the bacterial cell from plasmid entry, reducing or eliminating plasmid transformation. In some embodiments, expression of a Wadjet Type III defense system in bacteria protects the bacterial cell from plasmid entry, reducing or eliminating plasmid transformation.

In some embodiments, a microbial cell comprising a Wadjet Type I defense system protects the microbial cell from plasmid entry, reducing or eliminating plasmid transformation. In some embodiments, a microbial cell expressing a Wadjet Type I defense system protects the microbial cell from plasmid entry, reducing or eliminating plasmid transformation. In some embodiments, a microbial cell expressing a functional Wadjet Type I defense system protects the microbial cell from plasmid entry, reducing or eliminating plasmid transformation.

In some embodiments, a bacterial cell comprising a Wadjet Type I defense system protects the bacterial cell from plasmid entry, reducing or eliminating plasmid transformation. In some embodiments, a bacterial cell expressing a Wadjet Type I defense system protects the bacterial cell from plasmid entry, reducing or eliminating plasmid transformation. In some embodiments, a bacterial cell expressing a functional Wadjet Type I defense system protects the bacterial cell from plasmid entry, reducing or eliminating plasmid transformation.

In some embodiments, a microbial cell comprising a Wadjet Type II defense system protects the microbial cell from plasmid entry, reducing or eliminating plasmid transformation. In some embodiments, a microbial cell expressing a Wadjet Type II defense system protects the microbial cell from plasmid entry, reducing or eliminating plasmid transformation. In some embodiments, a microbial cell expressing a functional Wadjet Type II defense system protects the microbial cell from plasmid entry, reducing or eliminating plasmid transformation.

In some embodiments, a bacterial cell comprising a Wadjet Type II defense system protects the bacterial cell from plasmid entry, reducing or eliminating plasmid transformation. In some embodiments, a bacterial cell expressing a Wadjet Type II defense system protects the bacterial cell from plasmid entry, reducing or eliminating plasmid transformation. In some embodiments, a bacterial cell expressing a functional Wadjet Type II defense system protects the bacterial cell from plasmid entry, reducing or eliminating plasmid transformation.

In some embodiments, a microbial cell comprising a Wadjet Type III defense system protects the microbial cell from plasmid entry, reducing or eliminating plasmid transformation. In some embodiments, a microbial cell expressing a Wadjet Type III defense system protects the microbial cell from plasmid entry, reducing or eliminating plasmid transformation. In some embodiments, a microbial cell expressing a functional Wadjet Type III defense system protects the microbial cell from plasmid entry, reducing or eliminating plasmid transformation.

In some embodiments, a bacterial cell comprising a Wadjet Type III defense system protects the bacterial cell from plasmid entry, reducing or eliminating plasmid transformation. In some embodiments, a bacterial cell expressing a Wadjet Type III defense system protects the bacterial cell from plasmid entry, reducing or eliminating plasmid transformation. In some embodiments, a bacterial cell expressing a functional Wadjet Type III defense system protects the bacterial cell from plasmid entry, reducing or eliminating plasmid transformation.

In some embodiments, a Wadjet defense system provides a host cell with resistance to at least one phage. In some embodiments, a Wadjet Type I defense system provides a host cell with resistance to at least one phage. In some embodiments, a Wadjet Type II defense system provides a host cell with resistance to at least one phage. In some embodiments, a Wadjet Type III defense system provides a host cell with resistance to at least one phage.

In some embodiments, a Wadjet defense system (Defense system Xa, Xb, or Xc) provides a host cell with resistance to entry of conjugative elements. In some embodiments, a host cell expressing a function Wadjet defense system (Defense System Xa, Xb, or Xc) provides the host cell resistance from entry of conjugative elements.

As used herein, the term “a Wadjet anti-plasmid defense system” may be used interchangeably with the term “a Defense System X”, having all the same meanings and qualities. Further, in some embodiments, the term “a Wadjet Type I defense system” may be used interchangeably with the term “a Defense System Xa”, having all the same meanings and qualities, and in some embodiments, the term “a Wadjet Type II defense system” may be used interchangeably with the term “a Defense System Xb”, having all the same meanings and qualities, and in some embodiments, the term “a Wadjet Type III defense system” may be used interchangeably with the term “a Defense System Xc”, having all the same meanings and qualities.

A skilled artisan would appreciate that the term “Wadjet system” may be used interchangeably in some embodiments with “Wadjet defense system”, “Wadjet the defense system”, “Wadjet defense system”, “Wadjet anti-plasmid system”, “Wadjet Type I defense system”, “Wadjet Type II defense system”, “Wadjet Type III defense system”, “Defense System Xa”, “Defense System Xb”, and “Defense System Xc”.

In some embodiments, a microbial species does not comprise an endogenous Defense System X. In some embodiments, a microbial species does not express an endogenous Defense System X. In some embodiments, a microbial species does not express an endogenous functional Defense System X.

In some embodiments, a bacterial species does not comprise an endogenous Defense System X. In some embodiments, a bacterial species does not express an endogenous Defense System X. In some embodiments, a bacterial species does not express an endogenous functional Defense System X.

In some embodiments, a microbial species does not comprise an endogenous Defense System Xa. In some embodiments, a microbial species does not express an endogenous Defense System Xa. In some embodiments, a microbial species does not express an endogenous functional Defense System Xa.

In some embodiments, a bacterial species does not comprise an endogenous Defense System Xa. In some embodiments, a bacterial species does not express an endogenous Defense System Xa. In some embodiments, a bacterial species does not express an endogenous functional Defense System Xa.

In some embodiments, a microbial species does not comprise an endogenous Defense System Xb. In some embodiments, a microbial species does not express an endogenous Defense System Xb. In some embodiments, a microbial species does not express an endogenous functional Defense System Xb.

In some embodiments, a bacterial species does not comprise an endogenous Defense System Xb. In some embodiments, a bacterial species does not express an endogenous Defense System Xb. In some embodiments, a bacterial species does not express an endogenous functional Defense System Xb.

In some embodiments, a microbial species does not comprise an endogenous Defense System Xc. In some embodiments, a microbial species does not express an endogenous Defense System Xc. In some embodiments, a microbial species does not express an endogenous functional Defense System Xc.

In some embodiments, a bacterial species does not comprise an endogenous Defense System Xc. In some embodiments, a bacterial species does not express an endogenous Defense System Xc. In some embodiments, a bacterial species does not express an endogenous functional Defense System Xc.

A Wadjet defense system (Defense System X) comprises, in some embodiments, a nucleic acid construct comprising a nucleic acid construct comprising a nucleic acid sequence encoding a JetA polypeptide comprising a pfam11855 domain or a DUF3375 domain or a pfam09660 domain or a DUF2397 domain or no known domain, or any combination thereof; a JetB polypeptide comprising a pfam13835 domain or a DUF4194 domain or a pfam09661 domain or a DUF2398 domain or no known domain, or any combination thereof; a JetC polypeptide comprising a pfam13555 domain or a pfam13558 domain or a COG4913 domain or a COG1196 domain, or a combination thereof; and a JetD polypeptide comprising a pfam11795 domain or a DUF3322 domain or a pfam09983 domain or a DUF2220 domain or a pfam11796 domain or a DUF3323 domain or a pfam09664 domain or a DUF2399 domain or a COG4924 domain, or any combination thereof.

A Wadjet defense system (Defense System X) comprises, in some embodiments, a JetA polypeptide comprising a pfam11855 domain or a DUF3375 domain or a pfam09660 domain or a DUF2397 domain or no known domain, or any combination thereof; a JetB polypeptide comprising a pfam13835 domain or a DUF4194 domain or a pfam09661 domain or a DUF2398 domain or no known domain, or any combination thereof; a JetC polypeptide comprising a pfam13555 domain or a pfam13558 domain or a COG4913 domain or a COG1196 domain, or a combination thereof; and a JetD polypeptide comprising a pfam11795 domain or a DUF3322 domain or a pfam09983 domain or a DUF2220 domain or a pfam11796 domain or a DUF3323 domain or a pfam09664 domain or a DUF2399 domain or a COG4924 domain, or any combination thereof.

In some embodiments, a Wadjet Type I defense system (Defense System Xa) comprises a nucleic acid construct comprising a nucleic acid construct comprising a nucleic acid sequence encoding a JetA polypeptide comprising a pfam11855 domain or a DUF3375 domain, or any combination thereof; a JetB polypeptide comprising a pfam13835 domain or a DUF4194 domain, or any combination thereof; a JetC polypeptide comprising a pfam13555 domain or a pfam13558 domain or a COG4913 domain, or a combination thereof; and a JetD polypeptide comprising a pfam11795 domain or a DUF3322 domain or a pfam09983 domain or a DUF2220 domain or a COG4924 domain, or any combination thereof. In some embodiments, a Wadjet Type I defense system comprises a nucleic acid construct comprising a nucleic acid sequence comprising a jetA, jetB, jetC, jetD gene.

In some embodiments, a Wadjet Type I defense system (Defense System Xa) comprises a JetA polypeptide comprising a pfam11855 domain or a DUF3375 domain, or any combination thereof; a JetB polypeptide comprising a pfam13835 domain or a DUF4194 domain, or any combination thereof; a JetC polypeptide comprising a pfam13555 domain or a pfam13558 domain or a COG4913 domain, or a combination thereof; and a JetD polypeptide comprising a pfam11795 domain or a DUF3322 domain or a pfam09983 domain or a DUF2220 domain or a COG4924 domain, or any combination thereof. In some embodiments, a Wadjet Type I defense system comprises a JetA polypeptide, a JetB polypeptide, a JetC polypeptide, and a JetD polypeptide.

In some embodiments, a Defense System Xa comprises a JetA polypeptide comprising a pfam11855 domain or a DUF3375 domain or a combination thereof, or comprising an amino acid sequence having at least 80% identity to a sequence selected from the group referenced in Table 11 rows 2-2322 columns J and K; or a JetB polypeptide comprising a pfam13835 domain or a DUF4194 domain or a combination thereof, or comprising an amino acid sequence having at least 80% identity to a sequence selected from the group referenced in Table 11 rows 2-2322 columns N and O; or a JetC polypeptide comprising a pfam13555 domain or a pfam13558 domain or a COG4913 domain or a combination thereof, or comprising an amino acid sequence having at least 80% identity to a sequence selected from the group referenced in Table 11 rows 2-2322 columns R and S; or a JetD polypeptide comprising a pfam11795 domain or a DUF3322 domain or a pfam09983 domain or a DUF2220 domain or a COG4924 domain or any combination thereof, or comprising an amino acid sequence having at least 80% identity to a sequence selected from the group referenced in Table 11 rows 2-2322 columns V and W; or any combination thereof.

In some embodiments, a Defense System Xa comprises at least two different polypeptide components selected from a JetA polypeptide comprising a pfam11855 domain or a DUF3375 domain or a combination thereof, or comprising an amino acid sequence having at least 80% identity to a sequence selected from the group referenced in Table 11 rows 2-2322 columns J and K; a JetB polypeptide comprising a pfam13835 domain or a DUF4194 domain or a combination thereof, or comprising an amino acid sequence having at least 80% identity to a sequence selected from the group referenced in Table 11 rows 2-2322 columns N and O; a JetC polypeptide comprising a pfam13555 domain or a pfam13558 domain or a COG4913 domain or a combination thereof, or comprising an amino acid sequence having at least 80% identity to a sequence selected from the group referenced in Table 11 rows 2-2322 columns R and S; and a JetD polypeptide comprising a pfam11795 domain or a DUF3322 domain or a pfam09983 domain or a DUF2220 domain or a COG4924 domain or any combination thereof, or comprising an amino acid sequence having at least 80% identity to a sequence selected from the group referenced in Table 11 rows 2-2322 columns V and W. In some embodiments, a Defense System Xa comprises at least three different polypeptide components selected from a JetA polypeptide comprising a pfam11855 domain or a DUF3375 domain or a combination thereof, or comprising an amino acid sequence having at least 80% identity to a sequence selected from the group referenced in Table 11 rows 2-2322 columns J and K; a JetB polypeptide comprising a pfam13835 domain or a DUF4194 domain or a combination thereof, or comprising an amino acid sequence having at least 80% identity to a sequence selected from the group referenced in Table 11 rows 2-2322 columns N and O; a JetC polypeptide comprising a pfam13555 domain or a pfam13558 domain or a COG4913 domain or a combination thereof, or comprising an amino acid sequence having at least 80% identity to a sequence selected from the group referenced in Table 11 rows 2-2322 columns R and S; and a JetD polypeptide comprising a pfam11795 domain or a DUF3322 domain or a pfam09983 domain or a DUF2220 domain or a COG4924 domain or any combination thereof, or comprising an amino acid sequence having at least 80% identity to a sequence selected from the group referenced in Table 11 rows 2-2322 columns V and W. In some embodiments, a Defense System Xa comprises four different polypeptide components selected from a JetA polypeptide comprising a pfam11855 domain or a DUF3375 domain or a combination thereof, or comprising an amino acid sequence having at least 80% identity to a sequence selected from the group referenced in Table 11 rows 2-2322 columns J and K; a JetB polypeptide comprising a pfam13835 domain or a DUF4194 domain or a combination thereof, or comprising an amino acid sequence having at least 80% identity to a sequence selected from the group referenced in Table 11 rows 2-2322 columns N and O; a JetC polypeptide comprising a pfam13555 domain or a pfam13558 domain or a COG4913 domain or a combination thereof, or comprising an amino acid sequence having at least 80% identity to a sequence selected from the group referenced in Table 11 rows 2-2322 columns R and S; and a JetD polypeptide comprising a pfam11795 domain or a DUF3322 domain or a pfam09983 domain or a DUF2220 domain or a COG4924 domain or any combination thereof, or comprising an amino acid sequence having at least 80% identity to a sequence selected from the group referenced in Table 11 rows 2-2322 columns V and W.

In some embodiments, a Wadjet Type II defense system (Defense System Xb) comprises a nucleic acid construct comprising a nucleic acid construct comprising a nucleic acid sequence encoding a JetAⁱⁱ polypeptide comprising a pfam09660 domain or a DUF2397 domain, or any combination thereof; a JetBⁱⁱ polypeptide comprising a pfam09661 domain or a DUF2398 domain, or any combination thereof; a JetCⁱⁱ polypeptide comprising a pfam13558 domain; and a JetDⁱⁱ polypeptide comprising a pfam11796 domain or a DUF3323 domain or a pfam09664 domain or a DUF2399 domain, or any combination thereof. In some embodiments, a Wadjet Type II defense system comprises a nucleic acid construct comprising a nucleic acid sequence comprising a jetAⁱⁱ, jetBⁱⁱ, jetCⁱⁱ, jetDⁱⁱ gene.

In some embodiments, a Wadjet Type II defense system (Defense System Xb) comprises a JetAⁱⁱ polypeptide comprising a pfam09660 domain or a DUF2397 domain, or any combination thereof; a JetBⁱⁱ polypeptide comprising a pfam09661 domain or a DUF2398 domain, or any combination thereof; a JetCⁱⁱ polypeptide comprising a pfam13558 domain; and a JetDⁱⁱ polypeptide comprising a pfam11796 domain or a DUF3323 domain or a pfam09664 domain or a DUF2399 domain, or any combination thereof. In some embodiments, a Wadjet Type II defense system comprises a JetAⁱⁱ polypeptide, a JetBⁱⁱ polypeptides, a JetCⁱⁱ polypeptide, and a JetDⁱⁱ polypeptide.

In some embodiments, a Defense System Xb comprises a JetAⁱⁱ polypeptide comprising a pfam09660 domain or a DUF2397 domain or a combination thereof, or comprising an amino acid sequence having at least 80% identity to a sequence selected from the group referenced in Table 11 rows 2323-2844 columns J and K; or a JetBⁱⁱ polypeptide comprising a pfam09661 domain or a DUF2398 domain or a combination thereof, or comprising an amino acid sequence having at least 80% identity to a sequence selected from the group referenced in Table 11 rows 2323-2844 columns N and O; or a JetCⁱⁱ polypeptide comprising a pfam13558 domain or comprising an amino acid sequence having at least 80% identity to a sequence selected from the group referenced in Table 11 rows 2323-2844 columns R and S; or a JetDⁱⁱ polypeptide comprising a pfam11796 domain or a DUF3323 domain or a pfam09664 domain or a DUF2399 domain or a combination thereof, or comprising an amino acid sequence having at least 80% identity to a sequence selected from the group referenced in Table 11 rows 2323-2844 columns V and W; or any combination thereof.

In some embodiments, a Defense System Xb comprises at least two different polypeptide components selected from a JetAⁱⁱ polypeptide comprising a pfam09660 domain or a DUF2397 domain or a combination thereof, or comprising an amino acid sequence having at least 80% identity to a sequence selected from the group referenced in Table 11 rows 2323-2844 columns J and K; a JetBⁱⁱ polypeptide comprising a pfam09661 domain or a DUF2398 domain or a combination thereof, or comprising an amino acid sequence having at least 80% identity to a sequence selected from the group referenced in Table 11 rows 2323-2844 columns N and O; a JetCⁱⁱ polypeptide comprising a pfam13558 domain or comprising an amino acid sequence having at least 80% identity to a sequence selected from the group referenced in Table 11 rows 2323-2844 columns R and S; and a JetDⁱⁱ polypeptide comprising a pfam11796 domain or a DUF3323 domain or a pfam09664 domain or a DUF2399 domain or a combination thereof, or comprising an amino acid sequence having at least 80% identity to a sequence selected from the group referenced in Table 11 rows 2323-2844 columns V and W. In some embodiments, a Defense System Xb comprises at least three different polypeptide components selected from a JetAⁱⁱ polypeptide comprising a pfam09660 domain or a DUF2397 domain or a combination thereof, or comprising an amino acid sequence having at least 80% identity to a sequence selected from the group referenced in Table 11 rows 2323-2844 columns J and K; a JetBⁱⁱ polypeptide comprising a pfam09661 domain or a DUF2398 domain or a combination thereof, or comprising an amino acid sequence having at least 80% identity to a sequence selected from the group referenced in Table 11 rows 2323-2844 columns N and O; a JetCⁱⁱ polypeptide comprising a pfam13558 domain or comprising an amino acid sequence having at least 80% identity to a sequence selected from the group referenced in Table 11 rows 2323-2844 columns R and S; and a JetDⁱⁱ polypeptide comprising a pfam11796 domain or a DUF3323 domain or a pfam09664 domain or a DUF2399 domain or a combination thereof, or comprising an amino acid sequence having at least 80% identity to a sequence selected from the group referenced in Table 11 rows 2323-2844 columns V and W. In some embodiments, a Defense System Xb comprises four different polypeptide components selected from a JetAⁱⁱ polypeptide comprising a pfam09660 domain or a DUF2397 domain or a combination thereof, or comprising an amino acid sequence having at least 80% identity to a sequence selected from the group referenced in Table 11 rows 2323-2844 columns J and K; a JetBⁱⁱ polypeptide comprising a pfam09661 domain or a DUF2398 domain or a combination thereof, or comprising an amino acid sequence having at least 80% identity to a sequence selected from the group referenced in Table 11 rows 2323-2844 columns N and O; a JetCⁱⁱ polypeptide comprising a pfam13558 domain or comprising an amino acid sequence having at least 80% identity to a sequence selected from the group referenced in Table 11 rows 2323-2844 columns R and S; and a JetDⁱⁱ polypeptide comprising a pfam11796 domain or a DUF3323 domain or a pfam09664 domain or a DUF2399 domain or a combination thereof, or comprising an amino acid sequence having at least 80% identity to a sequence selected from the group referenced in Table 11 rows 2323-2844 columns V and W.

In some embodiments, a Wadjet Type III defense system (Defense System Xc) comprises a nucleic acid construct comprising a nucleic acid construct comprising a nucleic acid sequence encoding a JetDⁱⁱⁱ polypeptide comprising a pfam09664 domain or a DUF2399 domain or a pfam09983 domain or a DUF2220 domain, or any combination thereof; a JetAⁱⁱⁱ polypeptide comprising no known domain; a JetBⁱⁱⁱ polypeptide comprising no known domain; and a JetCⁱⁱⁱ polypeptide comprising a COG1196 domain. In some embodiments, a Wadjet Type III defense system (Defense System Xc) comprises a nucleic acid construct comprising a nucleic acid construct comprising a nucleic acid sequence encoding a JetDⁱⁱⁱ polypeptide comprising a pfam09664 domain or a DUF2399 domain or a pfam09983 domain or a DUF2220 domain, or any combination thereof; a JetAⁱⁱⁱ polypeptide; a JetBⁱⁱⁱ polypeptide; and a JetCⁱⁱⁱ polypeptide comprising a COG1196 domain, said JetAⁱⁱⁱ polypeptide being encoded by a gene positioned within 5 genes of a gene encoding JetDⁱⁱⁱ and or JetCⁱⁱⁱ in a genome of a prokaryote, and said JetBⁱⁱⁱ polypeptide being encoded by a gene positioned within 5 genes of a gene encoding JetDⁱⁱⁱ and or JetCⁱⁱⁱ in the genome of the prokaryote. In some embodiments, a Wadjet Type III defense system comprises a nucleic acid construct comprising a nucleic acid sequence comprising a jetDⁱⁱⁱ, jetAⁱⁱⁱ, jetBⁱⁱⁱ, and a jetCⁱⁱⁱ gene.

In some embodiments, a Wadjet Type III defense system (Defense System Xc) comprises a JetDⁱⁱⁱ polypeptide comprising a pfam09664 domain or a DUF2399 domain or a pfam09983 domain or a DUF2220 domain, or any combination thereof; a JetAⁱⁱⁱ polypeptide; a JetBⁱⁱⁱ polypeptide; and a JetCⁱⁱⁱ polypeptide comprising a COG1196 domain. In some embodiments, a Wadjet Type III defense system (Defense System Xc) comprises a JetDⁱⁱⁱ polypeptide comprising a pfam09664 domain or a DUF2399 domain or a pfam09983 domain or a DUF2220 domain, or any combination thereof; a JetAⁱⁱⁱ polypeptide; a JetBⁱⁱⁱ polypeptide; and a JetCⁱⁱⁱ polypeptide comprising a COG1196 domain, said JetAⁱⁱⁱ polypeptide being encoded by a gene positioned within 5 genes of a gene encoding JetDⁱⁱⁱ and or JetCⁱⁱⁱ in a genome of a prokaryote, and said JetBⁱⁱⁱ polypeptide being encoded by a gene positioned within 5 genes of a gene encoding JetDⁱⁱⁱ and or JetCⁱⁱⁱ in the genome of the prokaryote. In some embodiments, a Wadjet Type III defense system comprises a JetDⁱⁱⁱ polypeptide, a JetAⁱⁱⁱ polypeptide, a JetBⁱⁱⁱ polypeptide, and a JetCⁱⁱⁱ polypeptide.

In some embodiments, a Defense System Xc comprises a JetAⁱⁱⁱ polypeptide comprising an amino acid sequence having at least 80% identity to a sequence selected from the group referenced in Table 11 rows 2845-3174 columns J and K; or a JetBⁱⁱⁱ polypeptide comprising an amino acid sequence having at least 80% identity to a sequence selected from the group referenced in Table 11 rows 2845-3174 columns N and O; or a JetCⁱⁱⁱ polypeptide comprising a COG1196 domain or comprising an amino acid sequence having at least 80% identity to a sequence selected from the group referenced in Table 11 rows 2845-3174 columns R and S; or a JetDⁱⁱⁱ polypeptide comprising a pfam09983 domain or a pfam09664 domain or a DUF2220 or a DUF2399 domain or a combination thereof, or comprising an amino acid sequence having at least 80% identity to a sequence selected from the group referenced in Table 11 rows 2845-3174 columns V and W; or any combination thereof.

In some embodiments, a Defense System Xc comprises at least two different polypeptide components selected from a JetAⁱⁱⁱ polypeptide comprising an amino acid sequence having at least 80% identity to a sequence selected from the group referenced in Table 11 rows 2845-3174 columns J and K; a JetBⁱⁱⁱ polypeptide comprising an amino acid sequence having at least 80% identity to a sequence selected from the group referenced in Table 11 rows 2845-3174 columns N and O; a JetCⁱⁱⁱ polypeptide comprising a COG1196 domain or comprising an amino acid sequence having at least 80% identity to a sequence selected from the group referenced in Table 11 rows 2845-3174 columns R and S; and a JetDⁱⁱⁱ polypeptide comprising a pfam09983 domain or a pfam09664 domain or a DUF2220 or a DUF2399 domain or a combination thereof, or comprising an amino acid sequence having at least 80% identity to a sequence selected from the group referenced in Table 11 rows 2845-3174 columns V and W. In some embodiments, a Defense System Xc comprises at least three different polypeptide components selected from a JetAⁱⁱⁱ polypeptide comprising an amino acid sequence having at least 80% identity to a sequence selected from the group referenced in Table 11 rows 2845-3174 columns J and K; a JetBⁱⁱⁱ polypeptide comprising an amino acid sequence having at least 80% identity to a sequence selected from the group referenced in Table 11 rows 2845-3174 columns N and O; a JetCⁱⁱⁱ polypeptide comprising a COG1196 domain or comprising an amino acid sequence having at least 80% identity to a sequence selected from the group referenced in Table 11 rows 2845-3174 columns R and S; and a JetDⁱⁱⁱ polypeptide comprising a pfam09983 domain or a pfam09664 domain or a DUF2220 or a DUF2399 domain or a combination thereof, or comprising an amino acid sequence having at least 80% identity to a sequence selected from the group referenced in Table 11 rows 2845-3174 columns V and W. In some embodiments, a Defense System Xc comprises four different polypeptide components selected from a JetAⁱⁱⁱ polypeptide comprising an amino acid sequence having at least 80% identity to a sequence selected from the group referenced in Table 11 rows 2845-3174 columns J and K; a JetBⁱⁱⁱ polypeptide comprising an amino acid sequence having at least 80% identity to a sequence selected from the group referenced in Table 11 rows 2845-3174 columns N and O; a JetCⁱⁱⁱ polypeptide comprising a COG1196 domain or comprising an amino acid sequence having at least 80% identity to a sequence selected from the group referenced in Table 11 rows 2845-3174 columns R and S; and a JetDⁱⁱⁱ polypeptide comprising a pfam09983 domain or a pfam09664 domain or a DUF2220 or a DUF2399 domain or a combination thereof, or comprising an amino acid sequence having at least 80% identity to a sequence selected from the group referenced in Table 11 rows 2845-3174 columns V and W.

In some embodiments, a JetAⁱⁱⁱ polypeptide comprises identical domains as are present in JetA and JetAⁱⁱ. In some embodiments, a JetAⁱⁱⁱ polypeptide comprises homologous domains with domains present in JetA and JetAⁱⁱ, wherein the amino acid sequences comprise at least 80% homology. In some embodiments, a JetBⁱⁱⁱ polypeptide comprises identical domains as are present in JetB and JetBⁱⁱ. In some embodiments, a JetBⁱⁱⁱ polypeptide comprises homologous domains with domains present in JetB and JetBⁱⁱ, wherein the amino acid sequences comprise at least 80% homology.

In some embodiments, a Wadjet Type I defense system having an anti-plasmid activity comprises a nucleic acid construct comprising a nucleic acid sequence encoding a JetA polypeptide, a JetB polypeptide, a JetC polypeptide, and a JetD polypeptide. In some embodiments, a Wadjet Type I defense system having an anti-plasmid activity comprises a nucleic acid construct comprising a nucleic acid sequence comprising a jetA gene, a jetB gene, a jetC gene, and a jetD gene.

In some embodiments, a Wadjet Type II defense system having an anti-plasmid activity comprises a nucleic acid construct comprising a nucleic acid sequence encoding a JetAⁱⁱ polypeptide, a JetBⁱⁱ polypeptide, a JetCⁱⁱ polypeptide, and a JetDⁱⁱ polypeptide. In some embodiments, a Wadjet Type II defense system having an anti-plasmid activity comprises a nucleic acid construct comprising a nucleic acid sequence comprising a jetAⁱⁱ gene, a jetBⁱⁱ gene, a jetCⁱⁱ gene, and a jetDⁱⁱ gene.

In some embodiments, a Wadjet Type III defense system having an anti-plasmid activity comprises a nucleic acid construct comprising a nucleic acid sequence encoding a JetAⁱⁱⁱ polypeptide, a JetBⁱⁱⁱ polypeptide, a JetCⁱⁱⁱ polypeptide, and a JetDⁱⁱⁱ polypeptide. In some embodiments, a Wadjet Type III defense system having an anti-plasmid activity comprises a nucleic acid construct comprising a nucleic acid sequence comprising a jetAⁱⁱⁱ gene, a jetBⁱⁱⁱ gene, a jetCⁱⁱⁱ gene, and a jetDⁱⁱⁱ gene.

In some embodiments, the Wadjet Type I anti-plasmid defense system comprises a nucleic acid construct comprising nucleic acid sequences encoding a cassette comprising jetA, jetB, jetC, and jetD genes. In some embodiments, a construct comprising the Wadjet Type I defense system encodes one component of the defense system, whereby multiple constructs may be used to assemble the functional defense system.

In some embodiments, a Wadjet Type I (Defense System Xa) gene cassette comprises the nucleic acid sequence:

(SEQ ID NO: 17; construct 59 of Table 4)

| | |
|---|---|
| 1 | gttgatcaag aaacaactta tagcgtgtag tttttgttgg ataaaatttt aaaagttatg |
| 61 | taatttgatg caggggaatt gaaacgtaat atagaaattt ataattataa ttatagagtt |
| 121 | aaatgatggt gcttaatgga agacgatgat agcgaaaaag gaggctgaaa tgaaaaaact |
| 181 | atttgaatgc attccggaaa attatttcaa tttgtttgct gggaaaaatc gggggtttta |
| 241 | tgctgaagtt gcttttttac tatatgaaca attccatatc aatcgagctg gtattttata |
| 301 | cagtttgatg aaagatgagg ttcaagaatt aatagaaacg aaagaagagt taggagaatg |
| 361 | tattgatata gaagatgaaa tagaagaaaa agagcaagac agtgctggga gagcaaatga |
| 421 | ggtgcttcga cgtttaaaga aacttaaatg gattgatgta gaagtacgtg atcaatttga |
| 481 | agagtttata gtgcttccag tgtattctag ccgtattttg gctgtgctaa aggaaatttg |
| 541 | tgaaaataga acaattgaat atcaaagata ttgttttacg acatatcaat tattaacagg |
| 601 | agtggaggcg gaggagcgcc cagcttcagc tattttggag tctgaaaaat atagtgagca |
| 661 | actttatgat gaattaacaa ttttactgca caatatgaaa aatcatatgg aaacaattgc |
| 721 | agctaaagat gatatacaac aagtgttaga gcatcatttt ggagaatata aaaaagatat |
| 781 | aattgataaa agttaccatc gtttaaggac atcggatcat gtgtcgagat atcgaaataa |
| 841 | aatattggag cgtatacaga cttggttttt agatgataaa ttcatgaaac gtgctgccga |
| 901 | agatgctgta gaaagtggat attctgctaa ctgggaagaa gcaatggatg gattaagtaa |
| 961 | gaaaatgtat agggttgaag aaatttatag taatttagat gaaatttgta atcaaattga |
| 1021 | tgcacgccat tatcaatatt tacgagcagt tttagatcgt tctagatatt taagtactta |
| 1081 | taatgacagt atcaattaca aaatatctta tattttagaa catatagggc aaatggacga |
| 1141 | gaatatactc gattcttctc tgtttcgttt agtacagttg cggcaacttc aagaagcgtc |
| 1201 | actgttatcc ccacgtaaga aaaaaggtgt atatgaacca gaacagcatg aggttaatga |
| 1261 | aattagtgat gagatgcgag aggagctaaa ggctgaaaat ataagacgta tggaaaaggt |
| 1321 | tatgaatcga aagaaaatcc aaaaatttgt gttagaagca atgaaggggc gtcaagagat |
| 1381 | agagatgagt gaattgaatt tgcaaaatga ggatgacgct ttgtatttaa tttacgttta |
| 1441 | tttgtatggt tatagtaagg gaactggata caagcttagt gaagagccac ctgaatttat |
| 1501 | tacacaaaag gggtatacat tttctaatcg tacgataaaa cgagaaaatt agggggaagt |
| 1561 | tatgcaaaat gtatcggaaa gagaaagaga agagatgggg attgttgtta attatttatt |
| 1621 | ctcccataat tttttattga aagaatttga acgtgagaag tatcacctgg cagtacggaa |
| 1681 | taaagatatt attaagcaat atttacaggt aattggttgg gattttattg tggatgaaaa |
| 1741 | gcatgggtgt attgttattg tttcgccaca ttacgagcat cgtttgaaat tgaagaaaga |
| 1801 | cgaaacgatt tggttgttag tattaagact tatttatgag gaaaagcgta gcgctctatc |
| 1861 | tattagtcaa tatcccttta ccactcttca agaaatcaag gggaaatatg agacgtttcg |
| 1921 | attaccgttt gtatcaaaaa caaaattacg tgaacttgtg cagattggaa agcaaaatca |
| 1981 | acttttgaga ccaattgata atgacataga gtcggatgat tgtcgttttc agcttttcca |
| 2041 | ttcatgcatt catgtgctgc aacaaggaga tttaaatgta ttgtatgaaa aaataaaatc |
| 2101 | ttatagtgag ggtggggatc acagtgaaat ggatgaagaa actacgatta attaattggc |
| 2161 | attattatag tgatgaaaca attttatttg gaaaacaaac agtgatatct ggacatacag |
| 2221 | gagcagggaa atcgactgtt attgatgctt tacaagtcct atttatttcc gatgagcgga |
| 2281 | agattaaatt taattcagca gcatatgagg aggccaatcg tactttaatt aattatttgc |
| 2341 | gggggaagat aggtacagaa gaaaaacctt ttgtaagaga agggtatttt acaacatata |
| 2401 | ttgttgctga gttttatgat gagaaggcag gagagagttt tgtaattggt atttcaattg |
| 2461 | atgtatttaa agatgatgaa aaagtaaaag agtattttat tataccgaaa tctgagatta |
| 2521 | atatgatttc gttttttagt acaaaagacg agaaacgtta tgtggaaaag caagcagact |
| 2581 | tttgtaaaaa aataagagaa caatttccag aggctataat tgaaaaaagt tcaaatcaat |
| 2641 | atcaaaaggc tttattacaa cgttttggtg gattacatga aagatttgtt aaaacgtttg |
| 2701 | caagagcatt atcgtttaaa ccaatcgata acatgaaaga ttttgtatat aaaaatatat |
| 2761 | tggacgaaaa agaattaaaa atcgacgtta tgaggaatat ttttcaaaca catgaagaat |
| 2821 | tgcaaagaga attagaagaa ttaaaagaac gaaaggaaga gctggagcga atagataata |
| 2881 | tttatttgga atgtgtaaag ctggaagcgg atatttccat acaagaatat gttcttagag |
| 2941 | ggctagagta tttattaata caagaagaaa agagcatgtg taaaaaaagt attgaacaga |
| 3001 | gagaaaaaga attaagaaaa tgtgaatcag atcaaaaaaa gacagcggaa caaaaagagc |
| 3061 | atgctagaaa aaaagagact gagtatgaaa ttaagattaa ggatagtgct gaacaaaaaa |
| 3121 | gacaaaaaca attacaagaa caaatagctc aagcgaaaaa agaatgtggt gatttagagt |
| 3181 | gtaccaaaaa catatatgtt catagtttag caagagaaga aaaggatgtt tctagtcttt |
| 3241 | taaattatca aggtaatgaa tatttctctt taagcaagga cgagaagcat gcattggaaa |
| 3301 | taggaagaga ttgtctagcc ttcttgtcac ataatgatgg tactggaggg aatagggaag |
| 3361 | agcaaacatt aaataaactt ggagagagtt taaaaagaat tagtgggcgt ttttataaga |
| 3421 | gtacagcaga attggaacac agatctgctg aattgaaaac agaagagaaa gaattacttt |
| 3481 | ctgatataga gaatttaaaa agaaggaagc gtccttatcc catgtctgtt gaaaagttga |
| 3541 | agggattact agaaaaacat ttagaggatc aatcgaaggt atggattctt tgtgaagagt |
| 3601 | tagaaataaa aaatgataaa tggcgaaatg ccctagaagg gtatttgaat actcaaagat |
| 3661 | ttgatatttt ggtagaacca catatgtttg cgactgcact gtctatctac gaaaaggaaa |
| 3721 | agtggaatct tggtcttgaa ggtgttggac ttgttgatac tgaaaaagaa caaacatatt |
| 3781 | tagggaaagt tgagaagggt tctttagctg aagagatagt aggtggtaac ccaatagtac |
| 3841 | aggctcgaat tcatcattta ttaggtaggg ttataaaggc agacaatgaa caagaattac |
| 3901 | gaaagtataa aacagctgtt actgcgacat gtatgagtta tcaacgtctt gtagcacgcc |
| 3961 | aaattcctag aaaagtatat gaaaccccat atataggtgc acatgctatt caaaagcaat |
| 4021 | tagaaattaa agaagaaaac ttaaaggaaa ttcaaacaga actgcaaata gtaggatact |
| 4081 | atattaaaga ctttaagaaa tggattgaaa ttttagaaga taagcaatcg gattataaga |
| 4141 | attatatatt aaatttctca ttaaacgata gtatcttgga gtttaataag aacataaata |
| 4201 | aatggaagag tgaactaaat actcttgatt tgtcacgttt agaaagttta cgccaaaagt |
| 4261 | tgaaagagtg gaatggaaaa tataatcaat ttaatggaga agaaggtaga ctgtttgaac |
| 4321 | aaattgggaa ggtgaaggaa gaacttcaaa gagtgaatgc tgagttatgg aaaaaggaaa |
| 4381 | aggccgcaac agagatttta gagaagtgga aaaattggaa gtttgaatat agaatcgaat |
| 4441 | tattacaaga ggctgagcaa aggtacgagc aagctatttc tacaaataaa gcttatggtg |
| 4501 | ctataaaaaa taaatacgaa aataataaaa aagaaaatca aaataaatac gaagaaaaaa |
| 4561 | ggggttttct agaaagtgaa cgtaagtctt ataatgaagc gcgaactttc caaggaatta |
| 4621 | tacaggctaa agataataag cagtacgaag aagcgctccg gaaaattgca aacctcgata |
| 4681 | tcccaaagtt tgaacaagag atcaaggaaa cgttgcagca agctgaagag gaatttcagt |
| 4741 | ctcactttat ttacaagatg cgtgaggcta ttcaagcggc gcgtagagaa tttaatcaat |
| 4801 | tgaaccatgc gttaggaaga tttaaattcc gaaatgacac gtatcgattt gtaataaagc |
| 4861 | caagtgaaca atataaaaaa ttttatgatg taattatgga tgaaagagta cagccggaga |
| 4921 | tatctctatt tgattttggt gatgaagata gggcggagat tttaaaggac ttatttggta |
| 4981 | gattagtggt gggcgagtat ggtgaaaacg aagagtttgt ggactatcgt aattatctcg |
| 5041 | attttgattt aagcattaat aatgaaaatg gaacaagatt tatgtctaac ttattacgtg |
| 5101 | agcaatctgg tggagagacg caaacacctt tttatattgc catcctagca tcatttcaac |
| 5161 | acctttatcg aaataaaaat accattcgtt tagtagtgtt tgatgaggct tttaataaaa |
| 5221 | tggatgaaga gcggattcaa attagtttac gtcttattaa acagttggac ttgcaattaa |
| 5281 | ttgcagctgt gccagatgag aaaatggctc atatggcagc tgaggcagat acagctataa |
| 5341 | taattaaccg aatagggcat tcatgtttta ctgacatatt atcttatcca agggaggacg |
| 5401 | aggcaattgg attacaagag caagattctt tcagtcttat tgaataaata tgaaaatagt |
| 5461 | aaaactgctc atacaggtga gagaagtgct cagcgccctc aattttcatt tcggcaaaaa |
| 5521 | catgaacttt ctaaagcata taatgatgaa atggattata cgaaccgatt agaaattaat |
| 5581 | actgcattaa aggacttaat tcgtaaaaaa attattgaag tgaaatggga aaagtgggaa |
| 5641 | gagaatcgaa ttgctgaaaa ggtttattta cagtatgatt ttattccaca agcttataga |
| 5701 | gaagctggta tagaaccaaa aattgaaaag atgaatagga ttttaaaagt gttagaacca |
| 5761 | cttgctgttc attcatggga gtgggttcga caatggtata aagaagtaca acagtcgttt |
| 5821 | caaaataata aaacggcacg aattaattta aacgatgtta aaggatatga actacttgta |
| 5881 | aaagcacttt ctagattaga aggtttggaa gatagtatac cgaaaaggac ctttagccaa |
| 5941 | ttagtattcg gggatacaaa actttttgaa actacgatac aaaaccgatt attaataata |
| 6001 | tataaaaggt acggagatat agaatatgaa agtgataaag aatatttaga gagtattggg |
| 6061 | attttagaga atatccaacc tgtctatata aaaggaaatg ttgatattcg tgttagagga |
| 6121 | gagaagattg ctctcggttc ctttcctggt gggttcggtt taatggatga aacaataaaa |
| 6181 | gaactagaaa ttcagtatgt acacgatgaa agtataatgc ttattgaaaa catgaccaca |
| 6241 | tactatgaac aaattaaaaa aaataataat attttattta tctatacagg tgggtttcct |
| 6301 | aagaagaatg tacagcaatt gttaaaaaag ttgaatatat atttggaaaa tcatccagtt |
| 6361 | ccagtatatc actatggaga tctggactac ggaggaatcc aaatatttga gtacattaag |
| 6421 | cgttcgtttt tttcagggct tgagccgtat atgatggatg tagctaccta caggcaattt |
| 6481 | gtaaagtacg ggatggaatt tggtgaaggg tatgaagaaa aattactgaa gatgttagag |
| 6541 | aatgagcagt attctttatg gcatgagtta attaaagaaa tgcttaaaga aaagaagcgt |
| 6601 | gttgagcagg aagtgatagt tagaaacgta atttagtgta tttatataaa tttaaaaagg |
| 6661 | atgtctatag accaggataa cggtttatgg gcatttttta ttaggttttg tataaaagag |
| 6721 | atttagttag aggaaatgag atttaatatg ttttgtaaac gccataatat taacgcctga |
| 6781 | atttttttag gagaggaaag tagagtgttg gctttgtttt tcgtaattaa ctaagtaatg |
| 6841 | tgagaagttt tttatatggg gttatttata taattattaa gtgctagata atgaattgtt |
| 6901 | agtccatcct tattttggag gcatgttcta attaaataaa ctggttggat gtaataagtg |
| 6961 | aattcctggt aatttatcgt tggaaaaaaa tatatattat gtataattag tgtataaata |
| 7021 | aaattctctg aaatttgaaa ggtgataaaa. |

The coding regions for each of the jetA, jetB, jetC, and jetD gene sequences within this embodiment of a Wadjet Type I cassette (SEQ ID NO: 17) are as follows: nucleotides 146-1552 encode an embodiment of a JetA polypeptide, nucleotides 1562-2155 encode an embodiment of a JetB polypeptide, nucleotides 2133-5447 encode an embodiment of a JetC polypeptide, and nucleotides 5551-6636 encode an embodiment of a JetD polypeptide.

In some embodiments, a Defense System Xa comprising a JetA polypeptide, a JetB polypeptide, a JetC polypeptide, and a JetD polypeptide is encoded by the nucleic acid sequence set forth in SEQ ID NO: 17. In some embodiments, a Defense System Xa comprising a JetA polypeptide, a JetB polypeptide, a JetC polypeptide, and a JetD polypeptide, is encoded by a nucleic acid sequence having at least 80% homology to the sequence set forth in SEQ ID NO: 17. In some embodiments, a Defense System Xa comprising a JetA polypeptide, a JetB polypeptide, a JetC polypeptide, and a JetD polypeptide, is encoded by a nucleic acid sequence having at least 85% homology, or 90% homology, or 95% homology, or 97% homology, or 98% homology, or 99% homology to the sequence set forth in SEQ ID NO: 17. In some embodiments, a Defense System Xa comprising a JetA polypeptide, a JetB polypeptide, a JetC polypeptide, and a JetD polypeptide, is encoded by a nucleic acid sequence having at least 80% identity to the sequence set forth in SEQ ID NO: 17. In some embodiments, a Defense System Xa comprising a JetA polypeptide, a JetB polypeptide, a JetC polypeptide, and a JetD polypeptide, is encoded by a nucleic acid sequence having at least 85% identity, or 90% identity, or 95% identity, or 97% identity, or 98% identity, or 99% identity to the sequence set forth in SEQ ID NO: 17.

In some embodiments, the Wadjet Type II anti-plasmid defense system comprises a nucleic acid construct comprising nucleic acid sequences encoding a cassette comprising jetAⁱⁱ, jetBⁱⁱ, jetⁱⁱC, and jetⁱⁱD genes. In some embodiments, a construct comprising the Wadjet Type II defense system encodes one component of the defense system, whereby multiple constructs may be used to assemble the functional defense system.

In some embodiments, a Wadjet Type II (Defense System Xb) gene cassette comprises the nucleic acid sequence:

(SEQ ID NO: 18; construct 60 of Table 4)

| | |
|---|---|
| 1 | tctttgggtt cctttacttt ttgtaaaaaa tttaattctt tgcctgaagt atttcgaaaa |
| 61 | actgaaagcc tttaaaaaga atttcaccaa tcagtttaat tacagaccaa gattacaata |
| 121 | aatttaaaac cattcgatgt caatgtttaa atggaaaaac gtaagtatag gaatccttag |
| 181 | ttcaatatta tacaatggga ataatagaca aaaaatagga tgatgatcca tggattcaac |
| 241 | aatgaaaaaa ataatagagg ccagttattt aacagccgac tccgcggcac actatcgaac |
| 301 | gattcttcgc tatttttatc accagcacga aagaatgaga gattttattg caccggaaga |
| 361 | gttattggaa catatgcgtt cgatacctgc cttcgcggac tttcaagagg accagctcca |
| 421 | ccagcaatta gcgcagcttg taaaatggaa taatttaatt gccaggcagg atatgacaaa |
| 481 | cgcaaaaaca attgaagaat ataaaaagaa acggttccgt tatcaatgta caccctacac |
| 541 | ggttgagatt gagagaatga ttgtgcaact agaaaaatta ggagatacgt tccaagggtc |
| 601 | gttagaacgc tcccaatttg atcgcctttt ccaagcaata accagccttc aaaatgaact |
| 661 | agagaatgac ctaaacaaat cggcagaaga atatatgcgg atttgggagg acgtatttcg |
| 721 | ctattttcaa acgattcgta ccagcactgc tgattatatt gcttatatta atagtgaaca |
| 781 | aaccgatcaa cgaatgcaaa cagaggcatt tctagtctat aaaaatcagt ttacaacata |
| 841 | tttgcgagat tttattgtat ccttgcaaaa aacatcgctg caaatccagc attcattatc |
| 901 | agaattaacc ttggaacggt tacagcattt ttttcaaaaa ttaatcgagc atcgcggggc |
| 961 | aattccccgt cttgaggatg tttcgtcatc tacaaacgat tggcttactg aatatgagga |
| 1021 | atattggttt tcattacggc agtggttttt agggtcggct gttcagcaaa gtgaactaga |
| 1081 | tatccttcag tggcagacca atgaaatgat tcgccggatg acaagatacg ttcagcgcat |
| 1141 | cggtgaaagg cagcagcatt ttcgcagccg aaaaaaggac tatctgcaat tatcaaaatg |
| 1201 | gtttgttgaa tgcagggata gtgaggaagc tcataaactt tcagcagttg tattcggctc |
| 1261 | gatgacgatt caacatctcc agcttgagga agcgaccact gaaaaccttc atgtcgatac |
| 1321 | atgggatgag gcaccgactg aattaacgat aaaaccgaga acggtccgct accgtgaaaa |
| 1381 | aacaaaaccg ggatcgttta actcgaatga acagaagaaa aaagagcagc gagaactcta |
| 1441 | tttaaaggaa agagaacaag agaaaaagtt aatcgaaaaa tatatgaccc aaggtaaaat |
| 1501 | tacgctttca gcactctcga cagttgaacc attcatccgc aaggttttgt taagctggat |
| 1561 | tgggaaatca atggcggcca aaaatcgcat ggtcaagact gattacgggc ttcacgtaaa |
| 1621 | ggtaatgctt gattatgaga aaactattac cttacaagct gaggatggca atttactaat |
| 1681 | gcctgatgcg acattcttgt ttgaggagac taggtgatga taatggaaca aacgcaatta |
| 1741 | ttcgatgaaa aggcaatcca aggaatggac atcttgtttc atcattactg gatcttacgt |
| 1801 | gccgaacaac ctgaatggta tcagctgatt cgtgagcggg agaaggtatt gcgccgctat |
| 1861 | cttgatgaaa agttcggcct gcggctaatt gttcatcaac attttattaa actggaaaaa |
| 1921 | attcctgttg aaccggaagg ctggatgggc atccaagatt tccaagagcc aatggactat |
| 1981 | gcgatttttt gctgcgcact tgccttttta gaggggaaag cagtggatga acagtttttg |
| 2041 | ctttccgaac tatgtcaaga gattcaagcc gattatcctg gagattttcc gcttgattgg |
| 2101 | acgctctata cccaccggaa atcattgata agagctgtaa aagttttgat ggaatttcag |
| 2161 | ctcatacgaa ccattgatgg ggatatcggc cgcttcgatc aaaatgaaga gcaggaagtt |
| 2221 | ttatatgaag ctagcaccta tagtcgatat ttcatgagaa catatccaga tgatttttcg |
| 2281 | agctatcagc actggagcga gttactaaaa gaagattgga aattgaacca agaggatgaa |
| 2341 | cgtaggaagc gggtgtaccg caagctgttc ttttcacctg gtttgcatcg gctagatcaa |
| 2401 | caggatcctg attttctcta tatccggaac tatcgtaacc gcttagcaga agatattgaa |
| 2461 | aagcacagtg agtataaact ccatgtctat aaaaatacgg catttttatc gattgcggag |
| 2521 | ccaaggcaat atcaacaggt ttttcctaat tccaaggcat ccactgacat tatccttcag |
| 2581 | ttgtcaaagt acattcatgg ggagcctgag cgcttcaaag caaacgaaaa tggtgaaata |
| 2641 | ctgatgacgg aaggtgagtt tgaacaagtt gtggatgatt tacggcagca atttggaaca |
| 2701 | ggctgggcaa agtattttcg cgatatgagt acaaaaggaa ttcgaacgga actgcttcgt |
| 2761 | gccatgaagg attggatgat ggcggaagtg gattcagaaa cttctcttat tcggattaag |
| 2821 | tcattaacag gggtcatgac gggggaatat ccgagtgatt ttcaaacagg aggaacagaa |
| 2881 | tgatgacaga ggcaaaatgg gtgatgaacc gtgcggggtt attgaatttt tggtattacg |
| 2941 | atgatgaaat ttttcctttc tcggatggaa agctactgct taggggaacg aacggctccg |
| 3001 | ggaaatctgt aaccatgcaa agcttcctgc ctgttttgtt agacgggaaa aaatcacctg |
| 3061 | accgactcga tccatttgga tctaaagccc gcagaatgga agattattta ctcggggaaa |
| 3121 | aagaggtggt cgatcgtgat gagcgtacag ggtatctttt tatcgaatat aaaaaggccg |
| 3181 | gcgtagagcg atacattaca actgggatcg ggatgcaagc caagcggcat aaaggaatta |
| 3241 | agtcttggta ttttgtgatt accgataatc gcagaattgg atatgatttt gaacttgcac |
| 3301 | attcgcaact aggagaccga gttccttttt ccgcaaagga actggaaaat cggatcggag |
| 3361 | agggcggcta tgttgttcat acccagcgcg aatatatgga gctggtgaat aaatatattt |
| 3421 | ttggtttcca atcaaatgaa gcttatgaag acttgatcaa gcttttaatc caattacgaa |
| 3481 | gtcctaaact ttcgaaggat tttaaaccga cagtgattta tgaaatatta gagtcggcct |
| 3541 | tgcctccttt gaccgatgat gagttaaggc atctatctga taccattgaa agtatggatc |
| 3601 | aaacccagca gcagctcgaa cagttggaac gggaatttgc ctctagcagc aggctggtta |
| 3661 | accagtatca ttcctataat caatatattt tggccgaacg ggcagggaag tggcaggatg |
| 3721 | ccttaaaaag gtacactgtt gctgaggaac atgtaaaagg tttaacagcc caagacgaag |
| 3781 | aattgactca agaaattaaa caagaagagg aacagaagca gcaatttgcc cagcagcaag |
| 3841 | aaattgcctt agaagaaaaa aagcggttgg agcgccatga ggtttggaat ctggaagagg |
| 3901 | ataagcgtaa gaaaatagaa aatacaaaat ctttaagtag tgaaataaat tccttacaaa |
| 3961 | agaaatggga ccacaaaaac agtcagtaca atcgtctatg gcaggagcgg gagcagagtc |
| 4021 | aaaaccagat aaggcagcat gaaagcggca tggaagattt attaggggag ctgcaatttg |
| 4081 | atgcggaaga ggctgctttt tcagagcatg aggttaacgt ccatgatttc gaacgtcatc |
| 4141 | aggaggagga gtttgatttt tccatctgga ttggtgagat tggcagtcat gaacaattac |
| 4201 | ttgcgaattt aaatcaatta gctgatgagg aaaatcgtct gtcagaagag cataaccggc |
| 4261 | tccaacggca atcctctgag aaaaagaaag aggtagatgc aatccgcaag aatctcgatc |
| 4321 | atttagccga ttggtttacg gaagagaagc aaaggttgga gcatcaggta tttacttgga |
| 4381 | ttgagcagca tccaaagctt attttttcaa atgaaagacg ccaagaaatc gcccgatcaa |
| 4441 | tcgaaggttt atatgaagaa aatcggtacg agcaagtgag ggaaaaactc cttgctgtgg |
| 4501 | ttaatgatta tattaccgat atcagtacga agaaaaagct catggagaca aaaatagaag |
| 4561 | ataaaaagca cgaattggaa gcagcacgag cagaattgca tcactggaag acattaaaaa |
| 4621 | tgccaaaccc ggatcgtgct aaggatacag aggcatttcg cttgcagctg ctagaagacg |
| 4681 | ggcaggcatt tattccgttt tatgcggccg ttgagttcca agatgatgtg accgaagaac |
| 4741 | agaaagagcg gattgaatct gcgttaaaac aaacgggcat attggacagc ttgatcactg |
| 4801 | aaaatgccct tgccccaacc catgatcggg taattcgtcc tgagccgcaa ttactcggat |
| 4861 | atacgttagc agattatttg cgtccggatt tagaggcaga cagtcttatt tcaaacaaac |
| 4921 | ttgttgatga aatattacgg agcatctccc ttgaacagga aggggcaggt tttcatgtgg |
| 4981 | atgtcgatgg atcatattca cttggttgtt tagtggggca tgcaccaaat gaagggccgt |
| 5041 | ctaaatatat cggccgctca tcccgcaaac gataccagca agagaaaatc aaagaatgtc |
| 5101 | aggaaaccat tgagcaactc caattagagt tagaggagct aaaggtgcaa ctcagtcaat |
| 5161 | acgaagagaa tctcctccaa gcagcacaat ggaagcagac catgccaaca gatcaggaac |
| 5221 | ttaatgactt gaatgttcag attgaaaaga ccggccatca attggaggag caaaaaaagg |
| 5281 | ttctttttca gctggatgaa cagtggaagc aagtacacgg tcacctccaa gtgattaaaa |
| 5341 | tacagctcca tcaagaaggc cgccagctaa atctttcttt aactaaagaa gtcttagggc |
| 5401 | aagcattaat ctcagctaaa aactatcgtg atcaactcta tagctttaaa gatctttttc |
| 5461 | aaaaatgttt atttgcccgg aaaagaatag aggatttaac tcatcgctta tttgaaatgg |
| 5521 | aaacggaact ggacgatctt aaaggggatc aaaatgtcaa ggaatcacag ttgcggaaag |
| 5581 | aaaaggctga aattgagtca attgagcaac agctgaagct aaaagggatc gaggaagtaa |
| 5641 | gacttcggat tcaacaagtg cagcaggagc taagggaagc aacagaaggc atcaatcatc |
| 5701 | tacttgaaac cattcctcag aagaaagcaa aacaagaaac ctgtcaaaat gaattagcag |
| 5761 | cagctaaaac aagtgcggaa ttctggtcaa atatggcgga cgaatgggaa cagatggtga |
| 5821 | gagcggatat cgcccgtggt tttgtagaag ttgttgaaat ggatcctgtt aaaatcgtta |
| 5881 | aacagctgga atccatttta ggaaaatatg accgttcgaa gctaaatgaa cagctaacaa |
| 5941 | agacgttcat caatgaacag atatttttaa cggagtacag gatgtttgaa tacccggaag |
| 6001 | agaccgagcg tccagagtgg ttttcgaagg aatggggcga atactatgaa ccatttatga |
| 6061 | atgaatggaa tcagcttcaa agccgccgtt taattctaat ggaatataaa ggccagcgtg |
| 6121 | tcagtccgta ttttgtgttc acatcactgg aaaaagagtt agaagaccaa aaaggttggc |
| 6181 | ttgatgaaca agaccgccaa ctctatgagg atattatcgt caatacagtt ggagtaatct |
| 6241 | tacgaaatcg aatcaaacgt gccgaaaaat gggtgagtga gatggacaaa atcatggaaa |
| 6301 | gccgtgataa ttcatctgga ttaacgttct cgattgcatg gaaaccatta acggccgaat |
| 6361 | cggaacaaga attggataca aaagacttgg ttaagctttt gcagcggaat agtaagtttt |
| 6421 | tgaacgagga tgatttaaat cggattacaa aacacttcca atcgagaata ggaaaagcga |
| 6481 | aggaattaat tcagttacgg aatgaaggat cgacgcttca tcaagtgtta aaagaagtgt |
| 6541 | tggattatcg gaagtggttc acttttgttc tttcatttaa acgtgtaaat gaaccgaaac |
| 6601 | gagaattgac gaacaatgcc ttctttaaat ttagcggcgg cgagaaggca atggcgatgt |
| 6661 | atattccgtt gtttacggcg gcctattcaa gatataaaga agcaggagag atggcgccgt |
| 6721 | atatcatttc actcgatgag gcctttgccg gggtcgatga gaataatatc cgtgacatgt |
| 6781 | ttgaagtggt ggaacagctt ggctttaact atataatgaa ctcacaagcc ttatggggag |
| 6841 | attatgacac catttcaagt ctatcaattt gcgagttagt acggccaaag aatgccgact |
| 6901 | ttgttactgt gattcgctat caatgggacg ggaaacagcg cacctttgtg gtggacgatg |
| 6961 | agcacgttga ggagctagtg acacatgatt gacaaagtac aggtgtttag ggacgaacca |
| 7021 | ggttttgtta aattgtttac cctatttaaa gaaaaatacc gttcaatcgg gagaatcggc |
| 7081 | ggcatggtga gtttggatgg cttttcctat gatgaggtgg aatcaattgc cggctttttg |
| 7141 | ggacagtctg ttgaagcgct ccttgaaaaa gggaaggttt cattactttc ttttgaaaaa |
| 7201 | gaattaccgt tgaccggttt tgcagagtat tcccttattg aattgcttga agaagtgctc |
| 7261 | ggggagtcga tacttactaa acatgaggaa agtggcattg aagaagagaa agaaaagagg |
| 7321 | tttttacaag agttatcttt tatatgtcct gagggaagtt ggtggtggga ctggattgag |
| 7381 | tcgaaaccgc cggattcccg gtggatttgg tcgctttata agcaggatgc ggttggttta |
| 7441 | atggaaaaat tcatcaccgt ttttaaagca tatcaagatc ttccctgtaa ggagaataag |
| 7501 | tatgaaagac tgccgctgtt tgcccaacga acaacgggaa atccacatat tttcgataat |
| 7561 | aatcaactaa ctgggaaatt gctcgtcaat tgcctgcagg tagatcaaca gcttaagggg |
| 7621 | cgacgcgtac cgggaatgcc gaaaacaaca gaggatctaa atgatttgct aggtttgtat |
| 7681 | ggtttgatga gggatgattt gtggagtttt gtttcctgcc gcggattact tgcggaaggt |
| 7741 | gaaatgggga ttcatcctgt ttggaaagcc gcagctgaaa ctgacacagt tctaaacgtt |
| 7801 | ccgcttaagg aattactgaa aataaaaaga atctggcccg ctgcaggaaa aaaggtttgg |
| 7861 | attgttgaaa attcaagtgt ttgttcgacg attgttgatg aggttgctga tgctcctgta |
| 7921 | atctgcaccc acgggcaatt tcgggctgcg agctggatat tgttggagtt attagtggaa |
| 7981 | gcgggctgtc acctttacta ttcgggtgac ctagatccgg aaggggtatc gatggcacag |
| 8041 | cggcttttcg atcgttatca gggtcatgta acttgttgga ggatggatgt tgaatcctat |
| 8101 | gataaaacca tttcagatga ggatatttct ggcaggcttt ctaaattgga gtcaataact |
| 8161 | gcacctgaac ttcttgaagt agtgaatgtc cttaaaatgc ggcaaaaagc gggctatcaa |
| 8221 | gaaggtttag ttgagcaact tgttcaggat attaaaaacg agtttaatgt gttactttag |
| 8281 | gttgaatcaa atatagtagg aatccagttg gatccccacc acaaaaagcg ctat. |

The coding regions for each of the jetAⁱⁱ, jetBⁱⁱ, jetCⁱⁱ, and jetDⁱⁱ gene sequences within this embodiment of a Wadjet Type II cassette (SEQ ID NO: 18) are as follows: nucleotides 230-1717 encode an embodiment of a JetAⁱⁱ polypeptide, nucleotides 1717-2883 encode an embodiment of a JetBⁱⁱ polypeptide, nucleotides 2883-6992 encode an embodiment of a JetCⁱⁱ polypeptide, and nucleotides 6985-8280 encode an embodiment of a JetDⁱⁱ polypeptide.

In some embodiments, a Defense System Xb comprising a JetAⁱⁱ polypeptide, a JetBⁱⁱ polypeptide, a JetCⁱⁱ polypeptide, and a JetDⁱⁱ polypeptide is encoded by the nucleic acid sequence set forth in SEQ ID NO: 18. In some embodiments, a Defense System Xb comprising a JetAⁱⁱ polypeptide, a JetBⁱⁱ polypeptide, a JetCⁱⁱ polypeptide, and a JetDⁱⁱ polypeptide, is encoded by a nucleic acid sequence having at least 80% homology to the sequence set forth in SEQ ID NO: 18. In some embodiments, a Defense System Xb comprising a JetAⁱⁱ polypeptide, a JetBⁱⁱ polypeptide, a JetCⁱⁱ polypeptide, and a JetDⁱⁱ polypeptide, is encoded by a nucleic acid sequence having at least 85% homology, or 90% homology, or 95% homology, or 97% homology, or 98% homology, or 99% homology to the sequence set forth in SEQ ID NO: 18. In some embodiments, a Defense System Xb comprising a JetAⁱⁱ polypeptide, a JetBⁱⁱ polypeptide, a JetCⁱⁱ polypeptide, and a JetDⁱⁱ polypeptide, is encoded by a nucleic acid sequence having at least 80% identity to the sequence set forth in SEQ ID NO: 18. In some embodiments, a Defense System Xb comprising a JetAⁱⁱ polypeptide, a JetBⁱⁱ polypeptide, a JetCⁱⁱ polypeptide, and a JetDⁱⁱ polypeptide, is encoded by a nucleic acid sequence having at least 85% identity, or 90% identity, or 95% identity, or 97% identity, or 98% identity, or 99% identity to the sequence set forth in SEQ ID NO: 18.

In some embodiments, the Wadjet Type III anti-plasmid defense system comprises a nucleic acid construct comprising nucleic acid sequences encoding a cassette comprising jetAⁱⁱⁱ jetBⁱⁱⁱ, jetCⁱⁱⁱ, and jetDⁱⁱⁱ genes. In some embodiments, a construct comprising the Wadjet Type III defense system encodes one component of the defense system, whereby multiple constructs may be used to assemble the functional defense system.

In some embodiments, a Wadjet Type III (Defense System Xc) gene cassette comprises the nucleic acid sequence:

(SEQ ID NO: 19; construct 61 of Table 4)

| | |
|---|---|
| 1 | ttccgatact ctttagccat aaagcgatga attctgctgt tccgccgaat atcgcaacgg |
| 61 | ttagtgcata tggtaaacct acgcctagtg cacggatttc agttggaaag agttctgctt |
| 121 | ttacattgcg ttaatcgatg tgtaaccagt aacgatgatg aggccgacca tcatgagtga |
| 181 | aaatgctacc atgggttcta tatccatcat ttttaaaact tggtggatat ggagggacct |
| 241 | ggtactgttt tggttttgtg atgtctgtgg gatttcttgc tagcttctgc atgatttgtc |
| 301 | ggtaagtcga tatattcgga gttgcggtcg atataattgg ataatcgttg atatattttt |
| 361 | taaaggtatg gcattcattg gtagaaagag tgatgcgagc ttatgttaca atggaaatag |
| 421 | cttgtaagta aggaagtgac agtatgaatg gtatacaatt tgttcaattg tatgtaatga |
| 481 | aaagtgctga aaagattgat gagttgtata taaaaaaaga acaaggtatc acaactctcc |
| 541 | ccattataaa gcagaccccg cgtaagatta taaaaacagc ggaacttctt tttagtgaag |
| 601 | aagaggtttc ggatgtttct actactatta caaaaaactt ttattcacct aatgtaagga |
| 661 | aaagagaaag tgatgtttta aagtggttaa cgatacatga aatgatagat tgtatagaac |
| 721 | gagggatttt aattaaggaa gttcgttttg agaaggatgg gaaaactgtg cattccattt |
| 781 | tctatcgaat ggggtacggt ttgtttacat atatggaaaa gaaacaacag ctagaaaagg |
| 841 | taagagagga agaggcttta cagcaatgga tgaagcgcaa gcagtctatg ccgaagtaca |
| 901 | taaatgagta tacggaaaga ttgtggcatg ttttgtgcaa tattgaaagt gatttgtctt |
| 961 | ctatagatga aaaacgttgg tcgtttcaga agacgttttt atttttagag tttttacttg |
| 1021 | ctacatataa aatgagttgt gaaaagcgta cgtttgattg gaaagagatt ggtgcggcgt |
| 1081 | attatgaagt gattggtggt tctaaaaagt ttgatcaaca taaaaaagag tttcttaata |
| 1141 | agctggaaga tttgttagat gctccgccgc attgtttagg tttagtgagc ttagggacag |
| 1201 | tgacgcctat ctttttttgc gggaatttat atgaaggtaa tacgcgttat gagtgtgaca |
| 1261 | cagtgcgttc tttaacggat ttagttgtat ttcgtcatgc gtataaaacg gatgcgaagg |
| 1321 | tgttatggct cgttgaaaat agagcggtat taacgaggat tgccgcagag gatgatttta |
| 1381 | tttcaagtac aggtagtctc gttatcggag tggatgggca attacgtagt gggcataaaa |
| 1441 | ggttgattca aagcgtattg gaccactcaa aatctattac gaaaattatt atttggacgg |
| 1501 | attatgatgg ggcaggagag attattagtg ataagttatt cgaactagtt gctccatatg |
| 1561 | aacagtatac aagatggatt tcttcagaag gtaaggcgtt aaataaagat gaatttgagc |
| 1621 | aatccgttcg tttacgcgaa agtgaacaag aagagagttt aggaggtgtg gatgcttgga |
| 1681 | aggaatggat cagtcagtaa tttcgatttt gcaaacagat ccgtcgcata tgggagtacg |
| 1741 | aaatgcgatg aaagatttaa ataaattatc aggtgttatt tcagagcttg gagagagtga |
| 1801 | gtattttaat gatccattga aaacgtatga gtttctacta ttattaaata ttttaaatga |
| 1861 | agaagcgctt ggattgacgt cttctattga agatgtgcgt acgttgaaat tccgttaccg |
| 1921 | taatatgtac ggcggggatc cgtcaattcg tttagagcat ttcattaata ttttagataa |
| 1981 | atatggatgg atctcaaaag ggaaaagcaa gattacgatg atggacgttg ggaagcgaat |
| 2041 | gattgatgtg ttaattcgcc tagcaaacga ttcgttagcg tattacatgc aagatgaaat |
| 2101 | aacgaggtct ttatatcaag caaaccgtga tgcggattta agtgaagcgt atgatgataa |
| 2161 | aggtgtatcg ggcggaaata aattagcgag tatgataaaa aatgtagaag aagcggtaga |
| 2221 | aagactgaaa gagagagaac tcgaatattt agcggaccgc tatgcgttgc cgcaagtgca |
| 2281 | gattattcat cgtttaatga atgaattaaa tgttcgtttt gaagaaaggt tccaaaagtt |
| 2341 | tcaaacgttt gaagaaagtc ttgtgttaga gcctcttgtg aaaagaggaa cgagcgttat |
| 2401 | gtttgaaggt tcacaggtta gtcttggaac gattaagaag attttacatt ttacacatat |
| 2461 | tcagcagtct acagtcggag cgaatattcg tcatgatgcg cttcgtcgtt ttattgaaaa |
| 2521 | ttgttttacg aagcaaaata tggaattgcc agatgcacat gaaattttaa gttttatgga |
| 2581 | gcaagaccgt agagaaggcg agagacaaga tgggttatgg atgccagtac agtttgcggc |
| 2641 | gccgttatca agtgctgcga tttctagtgg tattcagttt ttagaagagt acgagccata |
| 2701 | tacggatcat atagaagaag ttgtgcagga agaatatgaa gagatagaag aaatgtcaga |
| 2761 | agcagaagtg aagaagctaa tggcagatca gcagtggagt atgacgaaag agcaaattca |
| 2821 | aacggaacga ttagaagagt ttcttatgga acaagaagaa gtcgatatgg aagagcttgt |
| 2881 | gcttgaagcc ggttctaata cgtggggaga tgcggtaaat gcattaatcg cactgtcagc |
| 2941 | tttaaccgct aataataagg cggatattat cgataatcaa aatgtaaaag aaagaaacat |
| 3001 | agaaaaagaa tgggaatggg gcagtgacga tgataaaaga aaacgagttc gaagccgaga |
| 3061 | aaggaattaa tccgctcgcg aaattaggag aacttcggta catattgtct cgtgatgaag |
| 3121 | agcatacgtt tatgcaactt ttattttcat cttcagcgcg tatgagagca acaaattttg |
| 3181 | gattgcgccg caaagaagta atgaaaatgc ttgggttaaa tagtgaggat atagaagggt |
| 3241 | ttgagtcatt tgtaatgcga ataaatggtg cgctaagtgg ctattttcag tgtgtatatg |
| 3301 | atgagagacg tgatcaagtc attgtgatga tgcgagtacc agcaaaacaa gcgagagaag |
| 3361 | tattatcttc agaaagttta ggattactta tgtttatttt ctaccatcaa gaagtgttgc |
| 3421 | aaaatgaatt tacattattt aatcaattac ttagtgcgct tggacatgaa acgttgcaag |
| 3481 | cacgtagaaa agtacaaaca aatttagatc cacttttaaa aattggggct attacgaaat |
| 3541 | atgattctcc gtcccaagaa gaagcatatg cgttaactgc cattggaagt agaatgtttt |
| 3601 | cagattcgtt cttacatagg gcggctgagt ttagtcagtc acaacaactt catatggatg |
| 3661 | atgttttaaa attctttaag cgttataacg taggaggtgg agagacgata tgattccata |
| 3721 | ccgtatttct ttttctggta tacgtgatta taagccgaga acgatttctt taatggggaa |
| 3781 | gcgtgatcac attttaattt caggtgcaaa tggtgccggg aaatcgacgc tgacattttg |
| 3841 | ctggggcgct gttatggcgt ctacgaaagt aaatgtggaa gggcttcgtt cgaaaaatct |
| 3901 | tccagacaat agagtatggc gagcgaaaat tgaacttata tatgaaaatg atggatttgt |
| 3961 | agatgcagca agatttgtga gatttacgct tgatttagaa caagagccag gtcatccatt |
| 4021 | gaaaaaagaa tattatattg cagaaggcga tcgggtagat gagtgggagc aagaaacgcg |
| 4081 | tttctcatct tcagataagc attttaactt tagagagtat aaaagaatgc ttcttcaaaa |
| 4141 | atataaaatt gatccagatg cgttctattt aatttggtac caacaagatg taaatcaatt |
| 4201 | tgcagtgatg aagcctgaag agaggttccg tatttttagc gagatgacag gaattgaaag |
| 4261 | tattcaaaag tcttgggagt ctttcaaaga acaagaacgt gataaggaag cgattttgca |
| 4321 | aacggctcgt aataaccaaa ttcaatataa gttcgaactt gagcgttggg agaaagagaa |
| 4381 | aaatcgttat gaagatcaac aaagccgtat aaaacgaggg attctgcaat ataaaacagc |
| 4441 | gttatgtaca ctagaagatc attattatac agaacgtatg aagtataagg atgagctaga |
| 4501 | agatgtgaaa gagaatcttg atgagaagta tgaagaagct cagcaaggag aaaaacagtt |
| 4561 | tgcacaatta gaggcggagt tactagcgaa acaagctgag tttcaaagtg cagaaaaaga |
| 4621 | gcgtgaaatt gccgaggaac gacaactaca gtgcaaaaag gaaagtaaag ataaagagcg |
| 4681 | agaatggaat gcaatgaaag aagagataaa agacgtaaca gatcgcgtga aaaatatagg |
| 4741 | aattagtgaa gatgttgtaa gagcagaaaa gagtagtaaa gagcagcaat tgcaagaagc |
| 4801 | tgaaaaggaa aaggtatata cagagcagct tattagtgag acaagttata atttaaatga |
| 4861 | attaaggtca gaagttgcga agctagaagt gcaagtagat aatgatgaag cagctgtcgt |
| 4921 | ttctgcaaag aattacatag tgcaatataa gtctagtttc tatatggaag aaaagcatac |
| 4981 | aaaagtagaa agtatgatga gacagacgaa agatcttgtt gaaacggtga agaaagatct |
| 5041 | tcagcaaaaa tacgaagaga aaaagcagct agagctaaat cgttacgtca gtccgaggca |
| 5101 | agaagaagga cttagatact taagaaggga agggttacaa gcttatccgc ttcgagactt |
| 5161 | aatagaatta gaaggcgacg cagaatttcg tcatgaagat attttaaata caattaaata |
| 5221 | tgcgatcttc gtagaatcaa aagaattcag accaccgaat gatttgtatt acgtaccgtt |
| 5281 | gccactcatt gttccaactg aaagtgttat ttccttaccg cagtataagc tgagaattaa |
| 5341 | agaaggagac aacgaaaaac ttgtttctgt agcaatgaaa gctttatggt gggtaaaaca |
| 5401 | attctttaca ggtgagcagc catgtattca aaagaatagt ttaattgata taagaggatt |
| 5461 | ccgtggggaa caagaaaaaa cggaatacat tttaagtgaa aaagcaatta tgcaacgttt |
| 5521 | agaagagaca ggaaaatgga ttgaagaaca tgaaaaacag ctcatccaat atgagcgtga |
| 5581 | tattcaaaca ctggaagaaa aagaaagaaa acttcacgac attgtatata aactaagaga |
| 5641 | tgcagaagcc gttttacaaa aagctgccga acgcacattt agaatcgaac agttagggcg |
| 5701 | aaaaacggaa gagaaacaaa ggctagaaca ggaaatacaa aggctatatg caattaacca |
| 5761 | agaattgcat acgcgcatat ctcgcttaag agagcgtctt gaaacgttag aacagtacga |
| 5821 | aatgatatat gaagaactgc ataaggaaaa agaaaaaatt gcacatatgc aaaagcttga |
| 5881 | acaaattcat agagagttat tggatgagct caagagagta gtagagcaaa gagatatact |
| 5941 | ggatgagact tgtaatagat tggaaattga atgtaataaa gcgagtcaaa aaacgaaaca |
| 6001 | acaaaaacaa gaagtagaag aactacgcgc gtatattcag cgtttagaaa agaaaaaaca |
| 6061 | agatgttcaa gagcatttca taacgacaga agaaaaacta attagcatac aaagagaaaa |
| 6121 | ccaggatgcg aatgacacgt atggaaaatt attagagcaa ctaggttggg aaaaacaaat |
| 6181 | agaagagtgg tctgaagcga aagcactttc tcaaaaacaa agcgcagaga ttacactaga |
| 6241 | aggagcactc ggtgaaacag ttgatccact agcgccagaa aattacgcga agatgaaaga |
| 6301 | agaaaatgaa aaaagtaatg cagaactgca taacgcagaa caaattttga acgaacttcg |
| 6361 | cgaaaatata gcacttatga gagaacgtct cgaaacgacg atccaaatga atgctcataa |
| 6421 | aatccataaa aagtttgaac agtacatgga acaattccac ttcgaaggaa aagtagaatg |
| 6481 | gaacatggac acaaacaaac acggtgacat gcgttactat ctctacataa aagcgcgtaa |
| 6541 | aaaaggtcac cgtggcaaaa tggaagacat tagcgccaaa ggacgcggag gcaaagtagg |
| 6601 | aagcggcgta tcaggaggag aagaatccct ttcatctcta ctattcgcac tagccttact |
| 6661 | acaaacaatc gaagcatccc caggctacat catcctagac gaatacgaca gcgccctaga |
| 6721 | cgacagccga aaagaaaaag tcttcaccct attcgaagag gaactaaatc ggaaaatgat |
| 6781 | catcgtctca ccaaaaagtc atgatccaaa ctacttacaa cacttctcgc aagcattaac |
| 6841 | agttatgcac gatgcgagtg tgccggttag tcgaattttg cagattaaga gggcggtaga |
| 6901 | agaaactgaa actgtaatca aggataacta atttaaaacg tccatatcaa attttgatat |
| 6961 | ggacgtttta ttgctgttta atatggagat aaagaaaata aggatgtgat tgtaaa. |

The coding regions for each of the jetAⁱⁱⁱ, jetBⁱⁱⁱ, jetCⁱⁱⁱ, and jetDⁱⁱⁱ gene sequences within this embodiment of a Wadjet Type III cassette (SEQ ID NO: 19) are as follows: nucleotides 444-1700 encode an embodiment of a JetDⁱⁱⁱ polypeptide, nucleotides 1676-3070 encode an embodiment of a JetAⁱⁱⁱ polypeptide, nucleotides 3030-3713 encode an embodiment of a JetBⁱⁱⁱ polypeptide, and nucleotides 3710-6931 encode an embodiment of a JetCⁱⁱⁱ polypeptide.

In some embodiments, a Defense System Xc comprising a JetAⁱⁱⁱ polypeptide, a JetBⁱⁱⁱ polypeptide, a JetCⁱⁱⁱ polypeptide, and a JetDⁱⁱⁱ polypeptide is encoded by the nucleic acid sequence set forth in SEQ ID NO: 19. In some embodiments, a Defense System Xc comprising a JetAⁱⁱⁱ polypeptide, a JetBⁱⁱⁱ polypeptide, a JetCⁱⁱⁱ polypeptide, and a JetDⁱⁱⁱ polypeptide, is encoded by a nucleic acid sequence having at least 80% homology to the sequence set forth in SEQ ID NO: 19. In some embodiments, a Defense System Xc comprising a JetAⁱⁱⁱ polypeptide, a JetBⁱⁱⁱ polypeptide, a JetCⁱⁱⁱ polypeptide, and a JetDⁱⁱⁱ polypeptide, is encoded by a nucleic acid sequence having at least 85% homology, or 90% homology, or 95% homology, or 97% homology, or 98% homology, or 99% homology to the sequence set forth in SEQ ID NO: 19. In some embodiments, a Defense System Xc comprising a JetAⁱⁱⁱ polypeptide, a JetBⁱⁱⁱ polypeptide, a JetCⁱⁱⁱ polypeptide, and a JetDⁱⁱⁱ polypeptide, is encoded by a nucleic acid sequence having at least 80% identity to the sequence set forth in SEQ ID NO: 19. In some embodiments, a Defense System Xc comprising a JetAⁱⁱⁱ polypeptide, a JetBⁱⁱⁱ polypeptide, a JetCⁱⁱⁱ polypeptide, and a JetDⁱⁱⁱ polypeptide, is encoded by a nucleic acid sequence having at least 85% identity, or 90% identity, or 95% identity, or 97% identity, or 98% identity, or 99% identity to the sequence set forth in SEQ ID NO: 19.

In some embodiments, the components of a Wadjet Type I defense system (Defense System Xa) comprise genes jetA, jetB, jetC, and jetD. In some embodiments, the components of a Wadjet Type I defense system consist of genes jetA, jetB, jetC, and jetD. In some embodiments, the components of a Wadjet Type I defense system comprise nucleic acid sequences encoding a JetA polypeptide, a JetB polypeptide, a JetC polypeptide, and a JetD polypeptide. In some embodiments, the components of a Wadjet Type I defense system consist of nucleic acid sequences encoding a JetA polypeptide, a JetB polypeptide, a JetC polypeptide, and a JetD polypeptide. In some embodiments, the components of a Wadjet Type I defense system comprise a jetB gene, a jetC gene, and a jetD gene. In some embodiments, the components of a Wadjet Type I defense system consist of a jetB gene, a jetC gene, and a jetD gene. In some embodiments, the components of a Wadjet Type I defense system comprise nucleic acid sequences encoding a JetB polypeptide, and a JetC polypeptide, and an MKsG polypeptide. In some embodiments, the components of a Wadjet Type I defense system consist of nucleic acid sequences encoding a JetB polypeptide, and a JetC polypeptide, and an MKsG polypeptide.

In some embodiments, the components of a Wadjet Type I defense system (Defense System Xa) comprise JetA, JetB, JetC, and JetD polypeptides. In some embodiments, the components of a Wadjet Type I defense system (Defense System Xa) consist of JetA, JetB, JetC, and JetD polypeptides. In some embodiments, the components of a Wadjet Type I defense system (Defense System Xa) comprise JetB, JetC, and JetD polypeptides. In some embodiments, the components of a Wadjet Type I defense system (Defense System Xa) consist of JetB, JetC, and JetD polypeptides. In some embodiments, the components of a Wadjet Type I defense system (Defense System Xa) comprise JetC, and JetD polypeptides. In some embodiments, the components of a Wadjet Type I defense system (Defense System Xa) consist of, and JetD polypeptides. In some embodiments, the components of a Wadjet Type I defense system (Defense System Xa) comprise a JetD polypeptide. In some embodiments, the components of a Wadjet Type I defense system (Defense System Xa) consist of a JetD polypeptide. In some embodiments, the components of a Wadjet Type I defense system (Defense System Xa) comprise JetA, JetC, and JetD polypeptides. In some embodiments, the components of a Wadjet Type I defense system (Defense System Xa) consist of JetA, JetC, and JetD polypeptides. In some embodiments, the components of a Wadjet Type I defense system (Defense System Xa) comprise JetA and JetD polypeptides. In some embodiments, the components of a Wadjet Type I defense system (Defense System Xa) consist of JetA and JetD polypeptides. In some embodiments, the components of a Wadjet Type I defense system (Defense System Xa) comprise JetA and JetC polypeptides. In some embodiments, the components of a Wadjet Type I defense system (Defense System Xa) consist of JetA and JetC polypeptides. In some embodiments, the components of a Wadjet Type I defense system (Defense System Xa) comprise JetB and JetC polypeptides. In some embodiments, the components of a Wadjet Type I defense system (Defense System Xa) consist of JetB and JetC polypeptides. In some embodiments, the components of a Wadjet Type I defense system (Defense System Xa) comprise JetB and JetD polypeptides. In some embodiments, the components of a Wadjet Type I defense system (Defense System Xa) consist of JetB and JetD polypeptides. In some embodiments, the components of a Wadjet Type I defense system (Defense System Xa) comprise JetB and JetC polypeptides. In some embodiments, the components of a Wadjet Type I defense system (Defense System Xa) consist of JetC and JetD polypeptides.

In some embodiments, the components of a Wadjet Type II defense system (Defense System Xb) comprise JetAⁱⁱ, JetBⁱⁱ, JetCⁱⁱ, and JetDⁱⁱ polypeptides. In some embodiments, the components of a Wadjet Type II defense system (Defense System Xb) consist of JetAⁱⁱ, JetBⁱⁱ, JetCⁱⁱ, and JetDⁱⁱ polypeptides. In some embodiments, the components of a Wadjet Type II defense system (Defense System Xb) comprise JetBⁱⁱ, JetCⁱⁱ, and JetDⁱⁱ polypeptides. In some embodiments, the components of a Wadjet Type II defense system (Defense System Xb) consist of JetBⁱⁱ, JetCⁱⁱ, and JetDⁱⁱ polypeptides. In some embodiments, the components of a Wadjet Type II defense system (Defense System Xb) comprise JetCⁱⁱ, and JetDⁱⁱ polypeptides. In some embodiments, the components of a Wadjet Type II defense system (Defense System Xb) consist of, and JetDⁱⁱ polypeptides. In some embodiments, the components of a Wadjet Type II defense system (Defense System Xb) comprise a JetDⁱⁱ polypeptide. In some embodiments, the components of a Wadjet Type II defense system (Defense System Xb) consist of a JetDⁱⁱ polypeptide. In some embodiments, the components of a Wadjet Type II defense system (Defense System Xb) comprise JetAⁱⁱ, JetCⁱⁱ, and JetDⁱⁱ polypeptides. In some embodiments, the components of a Wadjet Type II defense system (Defense System Xb) consist of JetAⁱⁱ, JetCⁱⁱ, and JetDⁱⁱ polypeptides. In some embodiments, the components of a Wadjet Type II defense system (Defense System Xb) comprise JetAⁱⁱ and JetDⁱⁱ polypeptides. In some embodiments, the components of a Wadjet Type II defense system (Defense System Xb) consist of JetAⁱⁱ and JetDⁱⁱ polypeptides. In some embodiments, the components of a Wadjet Type II defense system (Defense System Xb) comprise JetAⁱⁱ and JetCⁱⁱ polypeptides. In some embodiments, the components of a Wadjet Type II defense system (Defense System Xb) consist of JetAⁱⁱ and JetCⁱⁱ polypeptides. In some embodiments, the components of a Wadjet Type II defense system (Defense System Xb) comprise JetBⁱⁱ and JetCⁱⁱ polypeptides. In some embodiments, the components of a Wadjet Type II defense system (Defense System Xb) consist of JetBⁱⁱ and JetCⁱⁱ polypeptides. In some embodiments, the components of a Wadjet Type II defense system (Defense System Xb) comprise JetBⁱⁱ and JetDⁱⁱ polypeptides. In some embodiments, the components of a Wadjet Type II defense system (Defense System Xb) consist of JetBⁱⁱ and JetDⁱⁱ polypeptides. In some embodiments, the components of a Wadjet Type II defense system (Defense System Xb) comprise JetBⁱⁱ and JetCⁱⁱ polypeptides. In some embodiments, the components of a Wadjet Type II defense system (Defense System Xb) consist of JetCⁱⁱ and JetDⁱⁱ polypeptides.

In some embodiments, the components of a Wadjet Type III defense system (Defense System Xc) comprise JetAⁱⁱⁱ, JetBⁱⁱⁱ, JetCⁱⁱⁱ, and JetDⁱⁱⁱ polypeptides. In some embodiments, the components of a Wadjet Type III defense system (Defense System Xc) consist of JetAⁱⁱⁱ, JetBⁱⁱⁱ, JetCⁱⁱⁱ, and JetDⁱⁱⁱ polypeptides. In some embodiments, the components of a Wadjet Type III defense system (Defense System Xc) comprise JetBⁱⁱⁱ, JetCⁱⁱⁱ, and JetDⁱⁱⁱ polypeptides. In some embodiments, the components of a Wadjet Type III defense system (Defense System Xc) consist of JetBⁱⁱⁱ, JetCⁱⁱⁱ, and JetDⁱⁱⁱ polypeptides. In some embodiments, the components of a Wadjet Type III defense system (Defense System Xc) comprise JetCⁱⁱⁱ, and JetDⁱⁱⁱ polypeptides. In some embodiments, the components of a Wadjet Type III defense system (Defense System Xc) consist of, and JetDⁱⁱⁱ polypeptides. In some embodiments, the components of a Wadjet Type III defense system (Defense System Xc) comprise a JetDⁱⁱⁱ polypeptide. In some embodiments, the components of a Wadjet Type III defense system (Defense System Xc) consist of a JetDⁱⁱⁱ polypeptide. In some embodiments, the components of a Wadjet Type III defense system (Defense System Xc) comprise JetAⁱⁱⁱ, JetCⁱⁱⁱ, and JetDⁱⁱⁱ polypeptides. In some embodiments, the components of a Wadjet Type III defense system (Defense System Xc) consist of JetAⁱⁱⁱ, JetCⁱⁱⁱ, and JetDⁱⁱⁱ polypeptides. In some embodiments, the components of a Wadjet Type III defense system (Defense System Xc) comprise JetAⁱⁱⁱ and JetDⁱⁱⁱ polypeptides. In some embodiments, the components of a Wadjet Type III defense system (Defense System Xc) consist of JetAⁱⁱⁱ and JetDⁱⁱⁱ polypeptides. In some embodiments, the components of a Wadjet Type III defense system (Defense System Xc) comprise JetA¹¹ⁱ and JetCⁱⁱⁱ polypeptides. In some embodiments, the components of a Wadjet Type III defense system (Defense System Xc) consist of JetAⁱⁱⁱ and JetCⁱⁱⁱ polypeptides. In some embodiments, the components of a Wadjet Type III defense system (Defense System Xc) comprise JetBⁱⁱⁱ and JetCⁱⁱⁱ polypeptides. In some embodiments, the components of a Wadjet Type III defense system (Defense System Xc) consist of JetBⁱⁱⁱ and JetCⁱⁱⁱ polypeptides. In some embodiments, the components of a Wadjet Type III defense system (Defense System Xc) comprise JetBⁱⁱⁱ and JetDⁱⁱⁱ polypeptides. In some embodiments, the components of a Wadjet Type III defense system (Defense System Xc) consist of JetBⁱⁱⁱ and JetDⁱⁱⁱ polypeptides. In some embodiments, the components of a Wadjet Type III defense system (Defense System Xc) comprise JetBⁱⁱⁱ and JetCⁱⁱⁱ polypeptides. In some embodiments, the components of a Wadjet Type III defense system (Defense System Xc) consist of JetCⁱⁱⁱ and JetDⁱⁱⁱ polypeptides.

In some embodiments, a Wadjet system Type I, Type II, or Type III, comprising a non-functional JetA/JetAⁱⁱ/JetAⁱⁱⁱ polypeptide or lacking a JetA/JetAⁱⁱ/JetAⁱⁱⁱ polypeptide has increased transformation efficiency. In some embodiments, a Wadjet system Type I, Type II, or Type III, comprising a non-functional JetC/JetCⁱⁱ/JetCⁱⁱⁱ polypeptide or lacking a JetC/JetCⁱⁱ/JetCⁱⁱⁱ polypeptide has increased transformation efficiency. In some embodiments, a Wadjet system Type I, Type II, or Type III, comprising a non-functional JetD/JetDⁱⁱ/JetDⁱⁱⁱ polypeptide or lacking a JetD/JetDⁱⁱ/JetDⁱⁱⁱ polypeptide has increased transformation efficiency.

In some embodiments, a construct comprising the Wadjet defense system encodes more than one component of the defense system but less than all of the components required for a functionally active defense system, whereby multiple constructs may be used to assemble the functional defense system. For example, in some embodiments each of jetA, jetB, jetC, and jetD may be encoded by nucleic acid sequences comprised in different constructs, wherein expression of the combination of constructs produces a functional a Wadjet Type I defense system. In some embodiments each of jetAⁱⁱ, jetBⁱⁱ, jetCⁱⁱ, and jetDⁱⁱ may be encoded by nucleic acid sequences comprised in different constructs, wherein expression of the combination of constructs produces a functional a Type II Wadjet defense system. In some embodiments each of jetAⁱⁱⁱ, jetBⁱⁱⁱ, jetCⁱⁱⁱ, and jetDⁱⁱⁱ may be encoded by nucleic acid sequences comprised in different constructs, wherein expression of the combination of constructs produces a functional a Type III Wadjet defense system.

In some embodiments, components of a Wadjet Type I defense system (Defense Systems Xa) may be encoded by a nucleic acid encoding 1, 2, 3, or 4 components of the Wadjet defense system as described herein. For example, in some embodiments, a construct comprises a nucleic acid comprising a jetA gene. In some embodiments, a construct comprises a nucleic acid comprising a jetA gene and a jetB gene. In some embodiments, a construct comprises a nucleic acid comprising a jetA gene and a jetC gene. In some embodiments, a construct comprises a nucleic acid comprising a jetA gene and a jetD gene. In some embodiments, a construct comprises a nucleic acid comprising a jetB gene. In some embodiments, a construct comprises a nucleic acid comprising a jetB gene and a jetC gene. In some embodiments, a construct comprises a nucleic acid comprising a jetB gene and a jetD gene. In some embodiments, a construct comprises a nucleic acid comprising a jetC gene. In some embodiments, a construct comprises a nucleic acid comprising a jetC gene and a jetD gene. In some embodiments, a construct comprises a nucleic acid comprising a jetD gene. In some embodiments, a construct comprises a nucleic acid comprising a jetA gene, a jetB gene, and a jetC gene. In some embodiments, a construct comprises a nucleic acid comprising a jetA gene, a jetB gene, and a jetD gene. In some embodiments, a construct comprises a nucleic acid comprising a jetA gene, a jetC gene, and a jetD gene. In some embodiments, a construct comprises a nucleic acid comprising a jetD gene, a jetB gene, and a jetC gene. In some embodiments, a construct comprises a nucleic acid comprising a jetD gene, a jetB gene, a jetC gene, and a jetA gene.

In some embodiments, components of a Wadjet Type II defense system (Defense Systems Xb) may be encoded by a nucleic acid encoding 1, 2, 3, or 4 components of the Wadjet defense system as described herein. For example, in some embodiments, a construct comprises a nucleic acid comprising a jetAⁱⁱ gene. In some embodiments, a construct comprises a nucleic acid comprising a jetAⁱⁱ gene and a jetBⁱⁱ gene. In some embodiments, a construct comprises a nucleic acid comprising a jetAⁱⁱ gene and a jetDⁱⁱ gene. In some embodiments, a construct comprises a nucleic acid comprising a jetBⁱⁱ gene. In some embodiments, a construct comprises a nucleic acid comprising a jetBⁱⁱ gene and a jetCⁱⁱ gene. In some embodiments, a construct comprises a nucleic acid comprising a jetBⁱⁱ gene and a jetDⁱⁱ gene. In some embodiments, a construct comprises a nucleic acid comprising a jetCⁱⁱ gene. In some embodiments, a construct comprises a nucleic acid comprising a jetCⁱⁱ gene and a jetDⁱⁱ gene. In some embodiments, a construct comprises a nucleic acid comprising a jetDⁱⁱ gene. In some embodiments, a construct comprises a nucleic acid comprising a jetAⁱⁱ gene, a jetBⁱⁱ gene, and a jetCⁱⁱ gene. In some embodiments, a construct comprises a nucleic acid comprising a jetAⁱⁱ gene, a jetBⁱⁱ gene, and a jetDⁱⁱ gene. In some embodiments, a construct comprises a nucleic acid comprising a jetAⁱⁱ gene, a jetCⁱⁱ gene, and a jetDⁱⁱ gene. In some embodiments, a construct comprises a nucleic acid comprising a jetDⁱⁱ gene, a jetBⁱⁱ gene, and a jetCⁱⁱ gene. In some embodiments, a construct comprises a nucleic acid comprising a jetDⁱⁱ gene, a jetBⁱⁱ gene, a jetCⁱⁱ gene, and a jetAⁱⁱ gene.

In some embodiments, components of a Wadjet Type III defense system (Defense Systems Xc) may be encoded by a nucleic acid encoding 1, 2, 3, or 4 components of the Wadjet defense system as described herein. For example, in some embodiments, a construct comprises a nucleic acid comprising a jetAⁱⁱⁱ gene. In some embodiments, a construct comprises a nucleic acid comprising a jetAⁱⁱⁱ gene and a jetBⁱⁱⁱ gene. In some embodiments, a construct comprises a nucleic acid comprising a jetAⁱⁱⁱ gene and a jetCⁱⁱⁱ gene. In some embodiments, a construct comprises a nucleic acid comprising a jetAⁱⁱⁱ gene and a jetDⁱⁱⁱ gene. In some embodiments, a construct comprises a nucleic acid comprising a jetBⁱⁱⁱ gene. In some embodiments, a construct comprises a nucleic acid comprising a jetBⁱⁱⁱ gene and a jetCⁱⁱⁱ gene. In some embodiments, a construct comprises a nucleic acid comprising a jetBⁱⁱⁱ gene and a jetDⁱⁱⁱ gene. In some embodiments, a construct comprises a nucleic acid comprising a jetDⁱⁱⁱ gene. In some embodiments, a construct comprises a nucleic acid comprising a jetCⁱⁱⁱ gene and a jetDⁱⁱⁱ gene. In some embodiments, a construct comprises a nucleic acid comprising a jetDⁱⁱⁱ gene. In some embodiments, a construct comprises a nucleic acid comprising a jetAⁱⁱⁱ gene, a jetⁱⁱⁱB gene, and a jetCⁱⁱⁱ gene. In some embodiments, a construct comprises a nucleic acid comprising a jetAⁱⁱⁱ gene, a jetBⁱⁱⁱ gene, and a jetDⁱⁱⁱ gene. In some embodiments, a construct comprises a nucleic acid comprising a jetAⁱⁱⁱ gene, a jetCⁱⁱⁱ gene, and a jetDⁱⁱⁱ gene. In some embodiments, a construct comprises a nucleic acid comprising a jetDⁱⁱⁱ gene, a jetBⁱⁱⁱ gene, and a jetCⁱⁱⁱ gene. In some embodiments, a construct comprises a nucleic acid comprising a jetDⁱⁱⁱ gene, a jetBⁱⁱⁱ gene, a jetCⁱⁱⁱ gene, and a jetAⁱⁱⁱ gene.

In some embodiments, components of a Wadjet defense system (Defense System Xa, Xb, or Xc) may comprise a nucleic acid encoding 1, 2, 3, or 4 polypeptide components of the Wadjet defense system. For example, in some embodiments, a construct comprises a nucleic acid encoding a JetA or a JetAⁱⁱ or a JetAⁱⁱⁱ polypeptide, respectively. In some embodiments, a construct comprises a nucleic acid encoding a JetA or a JetAⁱⁱ or a JetAⁱⁱⁱ polypeptide, respectively and a JetB or a JetBⁱⁱ or a JetBⁱⁱⁱ polypeptide, respectively. In some embodiments, a construct comprises a nucleic acid encoding a JetA or a JetAⁱⁱ or a JetAⁱⁱⁱ polypeptide, respectively and a JetC or a JetCⁱⁱ or a JetCⁱⁱⁱ polypeptide, respectively. In some embodiments, a construct comprises a nucleic acid encoding a JetA or a JetAⁱⁱ or a JetAⁱⁱⁱ polypeptide, respectively and a JetD or a JetDⁱⁱ or a JetDⁱⁱⁱ polypeptide, respectively. In some embodiments, a construct comprises a nucleic acid encoding a JetB or a JetBⁱⁱ or a JetBⁱⁱⁱ polypeptide, respectively. In some embodiments, a construct comprises a nucleic acid encoding a JetB or a JetBⁱⁱ or a JetBⁱⁱⁱ polypeptide, respectively and a JetC or a JetCⁱⁱ or a JetCⁱⁱⁱ polypeptide, respectively. In some embodiments, a construct comprises a nucleic acid encoding a JetB or a JetBⁱⁱ or a JetBⁱⁱⁱ polypeptide, respectively and a JetD or a JetDⁱⁱ or a JetDⁱⁱⁱ polypeptide, respectively. In some embodiments, a construct comprises a nucleic acid encoding a JetC or a JetCⁱⁱ or a JetCⁱⁱⁱ polypeptide, respectively. In some embodiments, a construct comprises a nucleic acid encoding a JetC or a JetCⁱⁱ or a JetCⁱⁱⁱ polypeptide, respectively and a JetD or a JetDⁱⁱ or a JetDⁱⁱⁱ polypeptide, respectively. In some embodiments, a construct comprises a nucleic acid encoding a JetD or a JetDⁱⁱ or a JetDⁱⁱⁱ polypeptide, respectively. In some embodiments, a construct comprises a nucleic acid encoding a JetA or a JetAⁱⁱ or a JetAⁱⁱⁱ polypeptide, respectively, a JetB or a JetBⁱⁱ or a JetBⁱⁱⁱ polypeptide, respectively, and a JetC or a JetCⁱⁱ or a JetCⁱⁱⁱ polypeptide, respectively. In some embodiments, a construct comprises a nucleic acid encoding a JetA or a JetAⁱⁱ or a JetAⁱⁱⁱ polypeptide, a JetB or a JetBⁱⁱ or a JetBⁱⁱⁱ polypeptide, respectively, and a JetD or a JetDⁱⁱ or a JetDⁱⁱⁱ polypeptide, respectively. In some embodiments, a construct comprises a nucleic acid encoding a JetA or a JetAⁱⁱ or a JetAⁱⁱⁱ polypeptide, a JetC polypeptide, and a JetD or a JetDⁱⁱ or a JetDⁱⁱⁱ polypeptide, respectively. In some embodiments, a construct comprises a nucleic acid encoding a JetD or a JetDⁱⁱ or a JetDⁱⁱⁱ polypeptide, respectively, a JetB or a JetBⁱⁱ or a JetBⁱⁱⁱ polypeptide, respectively, and a JetC or a JetCⁱⁱ or a JetCⁱⁱⁱ polypeptide, respectively.

In some embodiments, the components making up a functional Wadjet Type I anti-plasmid defense system comprise a JetA polypeptide, a JetB polypeptide, a JetC polypeptide, and a JetD polypeptide, each encoded by a jetA, a jetB, a jetC, and a jetD gene, respectively.

In some embodiments, the components making up a functional Wadjet Type II anti-plasmid defense system comprise a JetAⁱⁱ polypeptide, a JetBⁱⁱ polypeptide, a JetCⁱⁱ polypeptide, and a JetDⁱⁱ polypeptide, each encoded by a jetAⁱⁱ, a jetBⁱⁱ, a jetCⁱⁱ, and a jetDⁱⁱ gene, respectively

In some embodiments, the components making up a functional Wadjet Type III anti-plasmid defense system comprise a JetAⁱⁱⁱ polypeptide, a JetBⁱⁱⁱ polypeptide, a JetCⁱⁱⁱ polypeptide, and a JetDⁱⁱⁱ polypeptide, each encoded by a jetAⁱⁱⁱ, a jetBⁱⁱⁱ, a jetCⁱⁱⁱ, and a jetDⁱⁱⁱ gene, respectively

In some embodiments, a Wadjet defense system having an anti-plasmid activity (Defense System Xa, Xb, or Xc) comprises a nucleic acid encoding a JetA or a JetAⁱⁱ or a JetAⁱⁱⁱ polypeptide, respectively. In some embodiments, a Wadjet defense system having an anti-plasmid activity (Defense System Xa, Xb, or Xc) comprise a nucleic acid encoding a JetA or a JetAⁱⁱ or a JetAⁱⁱⁱ polypeptide, respectively and a JetB or a JetBⁱⁱ or a JetBⁱⁱⁱ polypeptide, respectively. In some embodiments, a Wadjet defense system having an anti-plasmid activity (Defense System Xa, Xb, or Xc) comprise a nucleic acid encoding a JetA or a JetAⁱⁱ or a JetAⁱⁱⁱ polypeptide, respectively and a JetC or a JetCⁱⁱ or a JetCⁱⁱⁱ polypeptide, respectively. In some embodiments, a Wadjet defense system having an anti-plasmid activity (Defense System Xa, Xb, or Xc) comprise a nucleic acid encoding a JetA or a JetAⁱⁱ or a JetAⁱⁱⁱ polypeptide, respectively and a JetD or a JetDⁱⁱ or a JetDⁱⁱⁱ polypeptide, respectively. In some embodiments, a Wadjet defense system having an anti-plasmid activity (Defense System Xa, Xb, or Xc) comprise a nucleic acid encoding a JetA or a JetAⁱⁱ or a JetAⁱⁱⁱ polypeptide, respectively, a JetB or a JetBⁱⁱ or a JetBⁱⁱⁱ polypeptide, respectively, and a JetC or a JetCⁱⁱ or a JetCⁱⁱⁱ polypeptide, respectively. In some embodiments, a Wadjet defense system having an anti-plasmid activity (Defense System Xa, Xb, or Xc) comprise a nucleic acid encoding a JetA or a JetAⁱⁱ or a JetAⁱⁱⁱ polypeptide, respectively, a JetB or a JetBⁱⁱ or a JetBⁱⁱⁱ polypeptide, respectively, and a JetD or a JetDⁱⁱ or a JetDⁱⁱⁱ polypeptide, respectively. In some embodiments, a Wadjet defense system having an anti-plasmid activity (Defense System Xa, Xb, or Xc) comprise a nucleic acid encoding a JetA or a JetAⁱⁱ or a JetAⁱⁱⁱ polypeptide, respectively, a JetC or a JetCⁱⁱ or a JetCⁱⁱⁱ polypeptide, respectively, and a JetD or a JetDⁱⁱ or a JetDⁱⁱⁱ polypeptide, respectively. In some embodiments, a Wadjet defense system having an anti-plasmid activity (Defense System Xa, Xb, or Xc) comprise a nucleic acid encoding a JetB or a JetBⁱⁱ or a JetBⁱⁱⁱ polypeptide, respectively. In some embodiments, a Wadjet defense system having an anti-plasmid activity (Defense System Xa, Xb, or Xc) comprise a nucleic acid encoding a JetB or a JetBⁱⁱ or a JetBⁱⁱⁱ polypeptide, respectively and a JetC or a JetCⁱⁱ or a JetCⁱⁱⁱ polypeptide, respectively. In some embodiments, a Wadjet defense system having an anti-plasmid activity (Defense System Xa, Xb, or Xc) comprise a nucleic acid encoding a JetB or a JetBⁱⁱ or a JetBⁱⁱⁱ polypeptide, respectively and a JetD or a JetDⁱⁱ or a JetDⁱⁱⁱ polypeptide, respectively. In some embodiments, a Wadjet defense system having an anti-plasmid activity (Defense System Xa, Xb, or Xc) comprise a nucleic acid encoding a JetC or a JetCⁱⁱ or a JetCⁱⁱⁱ polypeptide, respectively. In some embodiments, a Wadjet defense system having an anti-plasmid activity (Defense System Xa, Xb, or Xc) comprise a nucleic acid encoding a JetC or a JetCⁱⁱ or a JetCⁱⁱⁱ polypeptide, respectively and a JetD or a JetDⁱⁱ or a JetDⁱⁱⁱ polypeptide, respectively.

In some embodiments, a Wadjet defense system having an anti-plasmid activity (Defense System Xa, Xb, or Xc) comprises a nucleic acid comprising a jetA, jetAⁱⁱ, or jetAⁱⁱⁱ gene, respectively. In some embodiments, a Wadjet defense system having an anti-plasmid activity (Defense System Xa, Xb, or Xc) comprises a nucleic acid comprising a jetA, jetAⁱⁱ, or jetAⁱⁱⁱ gene, respectively and a jetB, jetBⁱⁱ, or jetBⁱⁱⁱ gene, respectively. In some embodiments, a Wadjet defense system having an anti-plasmid activity (Defense System Xa, Xb, or Xc) comprises a nucleic acid comprising a jetA, jetAⁱⁱ, or jetAⁱⁱⁱ gene, respectively and a jetC, jetCⁱⁱ, or jetCⁱⁱⁱ gene, respectively. In some embodiments, a Wadjet defense system having an anti-plasmid activity (Defense System Xa, Xb, or Xc) comprises a nucleic acid comprising a jetA, jetAⁱⁱ, or jetAⁱⁱⁱ gene, respectively and a jetD, jetDⁱⁱ, or jetDⁱⁱⁱ gene, respectively. In some embodiments, a Wadjet defense system having an anti-plasmid activity (Defense System Xa, Xb, or Xc) comprises a nucleic acid comprising a jetA, jetAⁱⁱ, or jetAⁱⁱⁱ gene, respectively, a jetB, jetBⁱⁱ, or jetBⁱⁱⁱ gene, respectively, and a jetC, jetCⁱⁱ, or jetCⁱⁱⁱ gene, respectively. In some embodiments, a Wadjet defense system having an anti-plasmid activity (Defense System Xa, Xb, or Xc) comprises a nucleic acid comprising a jetA, jetAⁱⁱ, or jetAⁱⁱⁱ gene, respectively, a jetB, jetBⁱⁱ, or jetBⁱⁱⁱ gene, respectively, and a jetD, jetDⁱⁱ, or jetDⁱⁱⁱ gene, respectively gene. In some embodiments, a Wadjet defense system having an anti-plasmid activity comprise a nucleic acid comprising a jetA, jetAⁱⁱ, or jetAⁱⁱⁱ gene, respectively, a jetC, jetCⁱⁱ, or jetCⁱⁱⁱ gene, respectively, and a jetD gene. In some embodiments, a Wadjet defense system having an anti-plasmid activity (Defense System Xa, Xb, or Xc) comprises a nucleic acid comprising a jetB, jetBⁱⁱ, or jetBⁱⁱⁱ gene, respectively. In some embodiments, a Wadjet defense system having an anti-plasmid activity (Defense System Xa, Xb, or Xc) comprises a nucleic acid comprising a jetB, jetBⁱⁱ, or jetBⁱⁱⁱ gene, respectively and a jetC, jetCⁱⁱ, or jetCⁱⁱⁱ gene, respectively. In some embodiments, a Wadjet defense system having an anti-plasmid activity comprise a nucleic acid comprising a jetB, jetBⁱⁱ, or jetBⁱⁱⁱ gene, respectively and a jetD, jetDⁱⁱ, or jetDⁱⁱⁱ gene, respectively. In some embodiments, a Wadjet defense system having an anti-plasmid activity (Defense System Xa, Xb, or Xc) comprises a nucleic acid comprising a jetB, jetBⁱⁱ, or jetBⁱⁱⁱ gene, respectively, a jetD, jetDⁱⁱ, or jetDⁱⁱⁱ gene, respectively, and a jetC, jetCⁱⁱ, or jetCⁱⁱⁱ gene, respectively. In some embodiments, a Wadjet defense system having an anti-plasmid activity (Defense System Xa, Xb, or Xc) comprises a nucleic acid comprising a jetC, jetCⁱⁱ, or jetCⁱⁱⁱ gene, respectively. In some embodiments, a Wadjet defense system having an anti-plasmid activity (Defense System Xa, Xb, or Xc) comprises a nucleic acid comprising a jetC, jetCⁱⁱ, or jetCⁱⁱⁱ gene, respectively and a jetD, jetDⁱⁱ, or jetDⁱⁱⁱ gene, respectively. In some embodiments, a Wadjet defense system having an anti-plasmid activity (Defense System Xa, Xb, or Xc) comprises a nucleic acid comprising a jetD, jetDⁱⁱ, or jetDⁱⁱⁱ gene, respectively.

In some embodiments, a Wadjet Type I defense system having an anti-plasmid activity comprises a nucleic acid construct comprising nucleic acids encoding polypeptides in a set order. In one embodiment, the 5′ to 3′ order of polypeptides encoded is JetA, JetB, JetC, and JetD. In some embodiments the 5′ to 3′ order of polypeptides does not affect the anti-plasmid activity. In some embodiments the 5′ to 3′ order of polypeptides does affect the anti-plasmid activity.

In some embodiments, the 5′ to 3′ order of polypeptides is random comprising any order of JetA, JetB, JetC, and JetD. In some embodiments, for example but not limited to, the order is JetB, JetC, JetD, and JetA; or JetC, JetD, JetA, and JetB; or JetD, JetA, JetB, and JetC.

In some embodiments, a Wadjet Type I defense system having an anti-plasmid activity comprises a nucleic acid construct comprising genes in a set order. In some embodiment, the 5′ to 3′ order of genes is jetA, jetB, jetC, and jetD. In some embodiment, the 5′ to 3′ order of genes in a Wadjet defense system is not jetA, jetB, jetC, and jetD. In some embodiments the 5′ to 3′ order of genes does not affect the anti-plasmid activity. In some embodiments the 5′ to 3′ order of genes does affect the anti-plasmid activity.

In some embodiments, the 5′ to 3′ order of genes is random, for example any order of jetA, jetB, jetC, and jetD. In some embodiments, for example but not limited to, the order is jetB, jetC, jetD, and jetA; or jetC, jetD, jetA, and jetB; or jetD, jetA, jetB, and jetC.

In some embodiments, a Wadjet Type II defense system having an anti-plasmid activity comprises a nucleic acid construct comprising nucleic acids encoding polypeptides in a set order. In one embodiment, the 5′ to 3′ order of polypeptides encoded is JetAⁱⁱ, JetBⁱⁱ, JetCⁱⁱ, and JetDⁱⁱ. In some embodiments the 5′ to 3′ order of polypeptides does not affect the anti-plasmid activity. In some embodiments the 5′ to 3′ order of polypeptides does affect the anti-plasmid activity.

In some embodiments, the 5′ to 3′ order of polypeptides is random comprising any order of JetAⁱⁱ, JetBⁱⁱ, JetCⁱⁱ, and JetDⁱⁱ. In some embodiments, for example but not limited to, the order is JetBⁱⁱ, JetCⁱⁱ, JetDⁱⁱ, and JetAⁱⁱ; or JetCⁱⁱ, JetDⁱⁱ, JetAⁱⁱ, and JetBⁱⁱ; or JetDⁱⁱ, JetAⁱⁱ, JetBⁱⁱ, and JetCⁱⁱ.

In some embodiments, a Wadjet Type II defense system having an anti-plasmid activity comprises a nucleic acid construct comprising genes in a set order. In some embodiment, the 5′ to 3′ order of genes is jetAⁱⁱ, jetBⁱⁱ, jetCⁱⁱ, and jetDⁱⁱ. In some embodiment, the 5′ to 3′ order of genes in a Wadjet defense system is not jetAⁱⁱ, jetBⁱⁱ, jetCⁱⁱ, and jetDⁱⁱ. In some embodiments the 5′ to 3′ order of genes does not affect the anti-plasmid activity. In some embodiments the 5′ to 3′ order of genes does affect the anti-plasmid activity.

In some embodiments, the 5′ to 3′ order of genes is random, for example any order of jetAⁱⁱ, jetBⁱⁱ, jetCⁱⁱ, and jetDⁱⁱ. In some embodiments, for example but not limited to, the order is jetBⁱⁱ, jetCⁱⁱ, jetDⁱⁱ, and jetAⁱⁱ; or jetCⁱⁱ, jetDⁱⁱ, jetAⁱⁱ, and jetBⁱⁱ; or jetDⁱⁱ, jetAⁱⁱ, jetBⁱⁱ, and jetCⁱⁱ.

In some embodiments, a Wadjet Type III defense system having an anti-plasmid activity comprises a nucleic acid construct comprising nucleic acids encoding polypeptides in a set order. In one embodiment, the 5′ to 3′ order of polypeptides encoded is JetAⁱⁱⁱ, JetBⁱⁱⁱ, JetCⁱⁱⁱ, and JetDⁱⁱⁱ. In some embodiments the 5′ to 3′ order of polypeptides does not affect the anti-plasmid activity. In some embodiments the 5′ to 3′ order of polypeptides does affect the anti-plasmid activity.

In some embodiments, the 5′ to 3′ order of polypeptides is random comprising any order of JetAⁱⁱⁱ, JetBⁱⁱⁱ, JetCⁱⁱⁱ, and JetDⁱⁱⁱ. In some embodiments, for example but not limited to, the order is JetBⁱⁱⁱ, JetCⁱⁱⁱ, JetDⁱⁱⁱ, and JetAⁱⁱⁱ; or JetCⁱⁱⁱ, JetDⁱⁱⁱ, JetAⁱⁱⁱ, and JetBⁱⁱⁱ; or JetDⁱⁱⁱ, JetAⁱⁱⁱ, JetBⁱⁱⁱ, and JetCⁱⁱⁱ.

In some embodiments, a Wadjet Type I defense system having an anti-plasmid activity comprises a nucleic acid construct comprising genes in a set order. In some embodiment, the 5′ to 3′ order of genes is jetAⁱⁱⁱ, jetBⁱⁱⁱ, jetCⁱⁱⁱ, and jetDⁱⁱⁱ. In some embodiment, the 5′ to 3′ order of genes in a Wadjet defense system is not jetAⁱⁱⁱ, jetBⁱⁱⁱ, jetCⁱⁱⁱ, and jetDⁱⁱⁱ. In some embodiments the 5′ to 3′ order of genes does not affect the anti-plasmid activity. In some embodiments the 5′ to 3′ order of genes does affect the anti-plasmid activity.

In some embodiments, the 5′ to 3′ order of genes is random, for example any order of jetAⁱⁱⁱ, jetBⁱⁱⁱ, jetCⁱⁱⁱ, and jetDⁱⁱⁱ. In some embodiments, for example but not limited to, the order is jetBⁱⁱⁱ, jetCⁱⁱⁱ, jetDⁱⁱⁱ, and jetAⁱⁱⁱ; or jetCⁱⁱⁱ, jetDⁱⁱⁱ, jetAⁱⁱⁱ, and jetBⁱⁱⁱ; or jetDⁱⁱⁱ, jetAⁱⁱⁱ, jetBⁱⁱⁱ, and jetCⁱⁱⁱ.

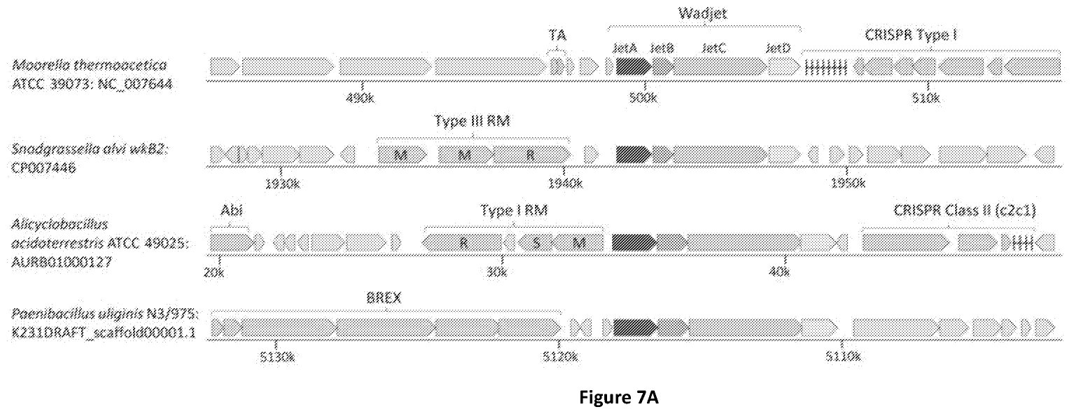

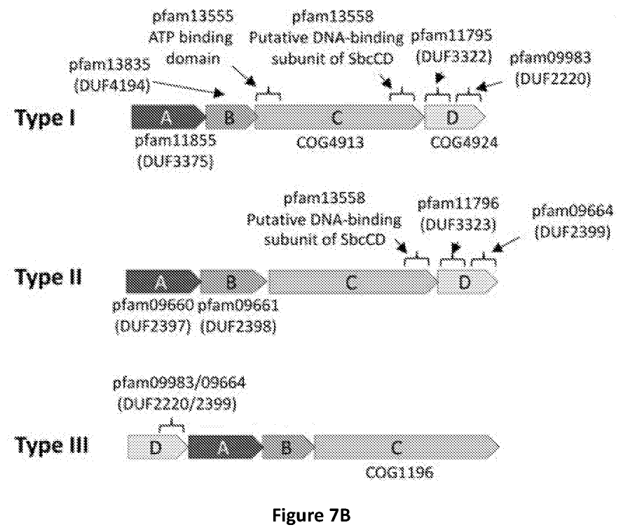

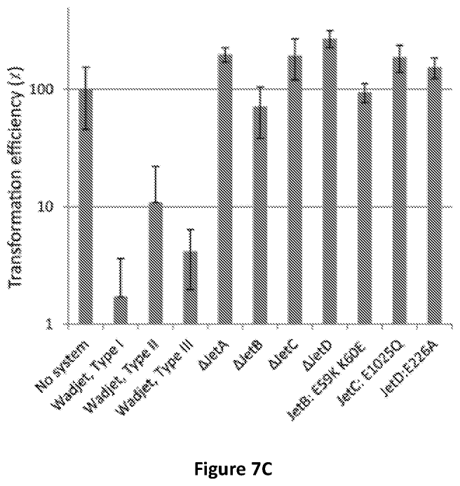

In some embodiments, the Wadjet Type I system (Defense System Xa) composition and order is as shown in FIG. 7B. In some embodiments, the Wadjet Type II system (Defense System Xb) composition and order is as shown in FIG. 7B. In some embodiments, the Wadjet Type III system (Defense System Xc) composition and order is as shown in FIG. 7B.

In some embodiments, a Wadjet Type I defense system having an anti-plasmid activity originates from a microbial genome, for example a bacterial or an archaeal genome (Table 11). In some embodiments, a Wadjet Type II defense system having an anti-plasmid activity originates from a microbial genome, for example a bacterial or an archaeal genome (Table 11). In some embodiments, a Wadjet Type III defense system having an anti-plasmid activity originates from a microbial genome, for example a bacterial or an archaeal genome (Table 11).

A skilled artisan would appreciate that a Wadjet system (Defense Xa, Xb, Xc) is not present in the majority of bacteria and or archaea species.

In some embodiments, a functional Wadjet defense system (Defense System Xa, Xb, or Xc) comprises a construct comprising nucleic acid sequences encoding polypeptides, wherein the nucleic acid sequence encoding each polypeptide may originate from a different microbial species. For example, the source of the nucleic acid sequence encoding a JetA, a JetAⁱⁱ, or a JetAⁱⁱⁱ polypeptides, respectively polypeptide may be one microbial species, the source of the nucleic acid sequence encoding a JetB, a JetBⁱⁱ, or a JetBⁱⁱⁱ polypeptides, respectively may be a different microbial species, the source of the nucleic acid sequence encoding a JetC, a JetCⁱⁱ, or a JetCⁱⁱⁱ polypeptides, respectively may be yet a different microbial species, and the source of the nucleic acid sequence encoding a JetD, a JetDⁱⁱ, or a JetDⁱⁱⁱ polypeptides, respectively may be still a different microbial species. In some embodiments, a functional Wadjet defense system (Defense System Xa, Xb, or Xc) comprises a non-naturally occurring combination of polypeptide components. In some embodiments, a functional Wadjet defense system (Defense System Xa, Xb, or Xc) comprises a combination of at least two polypeptides that do not naturally occur together. In some embodiments, a functional Wadjet defense system (Defense System Xa, Xb, or Xc) comprises a combination of at least three polypeptides that do not naturally occur together. In some embodiments, a functional Wadjet defense system (Defense System Xa, Xb, or Xc) comprises a combination of four polypeptides that do not naturally occur together.

In some embodiments, a functional Wadjet defense system (Defense System Xa, Xb, or Xc) comprises a construct comprising nucleic acid sequences encoding polypeptides, wherein the nucleic acid sequence encoding each polypeptide may originate from a different bacterial species. For example, the source of the nucleic acid sequence encoding a JetA, a JetAⁱⁱ, or a JetAⁱⁱⁱ polypeptides, respectively may be one bacterial species, the source of the nucleic acid sequence encoding a JetB, a JetBⁱⁱ, or a JetBⁱⁱⁱ polypeptides, respectively may be a different bacterial species, the source of the nucleic acid sequence encoding a JetC, a JetCⁱⁱ, or a JetCⁱⁱⁱ polypeptides, respectively may be yet a different bacterial species, and the source of the nucleic acid sequence encoding a JetD, a JetDⁱⁱ, or a JetDⁱⁱⁱ polypeptides, respectively may be still a different bacterial species.

In some embodiments, the source of the nucleic acid encoding a JetA, a JetAⁱⁱ, or a JetAⁱⁱⁱ polypeptides, respectively, a JetB, a JetBⁱⁱ, or a JetBⁱⁱⁱ polypeptides, respectively, a JetC, a JetCⁱⁱ, or a JetCⁱⁱⁱ polypeptides, respectively, and a JetD, a JetDⁱⁱ, or a JetDⁱⁱⁱ polypeptides, respectively is the same. In some embodiments, the source of the nucleic acid encoding a JetA, a JetAⁱⁱ, or a JetAⁱⁱⁱ polypeptides, respectively, a JetB, a JetBⁱⁱ, or a JetBⁱⁱⁱ polypeptides, respectively, a JetC, a JetCⁱⁱ, or a JetCⁱⁱⁱ polypeptides, respectively, and a JetD, a JetDⁱⁱ, or a JetDⁱⁱⁱ polypeptides, respectively is the not the same. In some embodiments, the source of some of the components is the same, while the source of other components is not the same. In some embodiments, the source of the nucleic acid sequence of some of the components is same and the source of other of the components is different. For example, but not limited to the source of any of the nucleic acid sequences encoding JetA, a JetAⁱⁱ, or a JetAⁱⁱⁱ polypeptides, respectively; JetB, a JetBⁱⁱ, or a JetBⁱⁱⁱ polypeptides, respectively; JetC, a JetCⁱⁱ, or a JetCⁱⁱⁱ polypeptides, respectively; and a JetD, a JetDⁱⁱ, or a JetDⁱⁱⁱ polypeptides, respectively may be the same or different, having any combination thereof. In some embodiments, the source of the nucleic acid sequence of any of the components of a Wadjet defense system (Defense Systems Xa, Xb, or Xc) comprises any of the species listed in Table 11.

In some embodiments, a Wadjet defense system (Defense Systems Xa, Xb, or Xc) having an anti-plasmid activity comprises a nucleic acid construct comprising non-coding regions between the nucleic acid sequences encoding the polypeptides. In some embodiments, the non-coding regions between the nucleic acid sequences comprises nucleic acid sequence not naturally occurring in the microbial species of origin. In some embodiments, the non-coding regions between the nucleic acid sequences comprises nucleic acid sequence is the naturally occurring in the microbial species of origin.

In some embodiments, a Wadjet system disclosed herein (Defense Systems Xa, Xb, or Xc) comprises a multi-gene phage resistance system broadly distributed in microbial genomes. According to some embodiments, the Wadjet system (Defense Systems Xa, Xb, or Xc) components are located in a gene cluster in a microbial cell genome. According to some embodiments, the Wadjet system (Defense Systems Xa, Xb, or Xc) components are located in close proximity (e.g. positioned within 20 genes, 10 genes, 5 genes or less one from the other) in a genome of a prokaryotic cell. According to some embodiments the prokaryotic cell expresses an endogenous Wadjet defense system (Defense Systems Xa, Xb, or Xc). According to some embodiments, the prokaryotic cell expresses an endogenous functional Wadjet defense system (Defense Systems Xa, Xb, or Xc). According to some embodiments, the species of prokaryotic cell is selected from the group consisting of the species listed in Table 11. According to some embodiments, a prokaryotic cell expresses a non-endogenous Wadjet defense system (Defense Systems Xa, Xb, or Xc). According to some embodiments, a prokaryotic cell expresses a non-endogenous functional Wadjet defense system (Defense Systems Xa, Xb, or Xc). According to some embodiments, the species of prokaryotic cell expressing a non-endogenous functional Wadjet defense system (Defense Systems Xa, Xb, or Xc) is selected from the group consisting of the species listed in Table 11.

In some embodiments, Wadjet Type I defense system components comprise JetA, JetB, JetC and JetD polypeptides. In some embodiments, Wadjet Type I defense system components comprise functional portions of JetA, JetB, JetC and JetD polypeptides. In some embodiments, the Wadjet Type I defense system components are encoded by jetA, jetB, jetC and jetD genes. In some embodiments, Wadjet Type I defense system components comprise JetB, JetC and JetD polypeptides.

In some embodiments, Wadjet Type II defense system components comprise JetAⁱⁱ, JetBⁱⁱ, JetCⁱⁱ and JetDⁱⁱ polypeptides. In some embodiments, Wadjet Type II defense system components comprise functional portions of JetAⁱⁱ, JetBⁱⁱ, JetCⁱⁱ and JetDⁱⁱ polypeptides. In some embodiments, the Wadjet Type II defense system components are encoded by jetAⁱⁱ, jetBⁱⁱ, jetCⁱⁱ and jetDⁱⁱ genes.

In some embodiments, Wadjet Type III defense system components comprise JetAⁱⁱⁱ, JetBⁱⁱⁱ, JetCⁱⁱⁱ and JetDⁱⁱⁱ polypeptides. In some embodiments, Wadjet Type III defense system components comprise functional portions of JetAⁱⁱⁱ, JetBⁱⁱⁱ, JetCⁱⁱⁱ and JetDⁱⁱⁱ polypeptides. In some embodiments, the Wadjet Type III defense system components are encoded by jetAⁱⁱⁱ, jetBⁱⁱⁱ, jetCⁱⁱⁱ and jetDⁱⁱⁱ genes.

Non-limiting embodiments of endogenous Wadjet systems (Defense Systems Xa, Xb, and Xc) and the respective location of their components are provided in Table 11 herein.

In some embodiments, the components of a Wadjet system may be identified by the presence of similar domains present within each component. In some embodiments, domains comprise pfam domains and or clusters of orthologous groups (COG) domains.

In some embodiments, the term “JetA” refers to the polynucleotide or expression product e.g., the polypeptide encoded by the jetA gene, comprising variants jetA, jetAⁱⁱ and jetAⁱⁱⁱ, associated with Defense Systems Xa, Xb, and Xc, respectively. In some embodiments, the terms “jetA”, “jetAⁱⁱ” and “jetAⁱⁱⁱ”, may be used interchangeably. In some embodiments, the term “JetA” refers to a JetA polypeptide or a JetAⁱⁱ polypeptide or a JetAⁱⁱⁱ polypeptide (FIG. 7B). In some embodiments, the terms JetA, JetAⁱⁱ, and JetAⁱⁱⁱ may be used interchangeably.

A skilled artisan would recognize that each Defense System X (Xa, Xb, and Xc) comprises a JetA polypeptide (A), a JetB polypeptide (B), a Jet C polypeptide (C), and a JetD polypeptide (C), wherein the polypeptides are encoded by a jetA gene, a jetB gene, a jetC gene, or a jetD gene, and variants thereof.

In some embodiments, the jetA gene encodes a polypeptide comprising a pfam11855 domain. In some embodiments, the jetA gene encodes a polypeptide comprising a DUF3375 domain. In some embodiments, the jetA gene encodes a polypeptide comprising a pfam09660 domain. In some embodiments, the jetA gene encodes a polypeptide comprising a DUF2397 domain. In some embodiments, the jetA gene encodes a polypeptide comprising no known domain. In some embodiments, the jetA gene encodes a polypeptide comprising a pfam11855 domain, or a DUF3375 domain, or a pfam09660 domain, or a DUF2397 domain, or any combination thereof. In some embodiments, the jetA gene encodes a polypeptide comprising a pfam11855 domain and a DUF3375 domain. In some embodiments, a jetA gene encodes a polypeptide comprising a pfam09660 domain and a DUF2397 domain.

In some embodiments, the jetA gene encodes a polypeptide comprising a member of the MukF-like family. MukF appears to play a role in chromosome partitioning during cell division. MukF has been described as a member of the kleisin family, which includes proteins that commonly mediate the interaction between SMCs and other accessory proteins.

In some embodiments, JetA polypeptide is encoded by a gene positioned within 5-60 genes of a gene encoding a JetB, JetC, and or JetD polypeptide in a genome of a prokaryotic cell. In some embodiments, JetA polypeptide is encoded by a gene positioned within 5 genes upstream (5′) or downstream (3′) to a gene encoding a JetB, JetC, and or JetD polypeptide in a genome of a prokaryotic cell. In some embodiments, JetA polypeptide is encoded by a gene positioned within 5 genes upstream (5′) and downstream (3′) to a gene encoding a JetB, JetC, and or JetD polypeptide, or any combination thereof, in a genome of a prokaryotic cell.

In some embodiments, JetA and JetB are encoded by genes positioned sequentially 5′ to 3′ in a genome of a prokaryotic cell. In some embodiments, JetA and JetB are encoded by genes positioned contiguously 5′ to 3′ in a genome of a prokaryotic cell. In some embodiments, JetA, JetB, and JetC are encoded by genes positioned sequentially 5′ to 3′ in a genome of a prokaryotic cell. In some embodiments, JetA, JetB, and JetC are encoded by genes positioned contiguously 5′ to 3′ in a genome of a prokaryotic cell. In some embodiments, JetA, JetB, JetC, and JetD are encoded by genes positioned sequentially 5′ to 3′ in a genome of a prokaryotic cell. In some embodiments, JetA, JetB, JetC, and JetD are encoded by genes positioned contiguously 5′ to 3′ in a genome of a prokaryotic cell. In some embodiments, JetD, JetA JetB, and JetC are encoded by genes positioned sequentially 5′ to 3′ in a genome of a prokaryotic cell. In some embodiments, JetD, JetA, JetB, and JetC are encoded by genes positioned contiguously 5′ to 3′ in a genome of a prokaryotic cell.

In some embodiments, jetA and jetB genes are positioned sequentially 5′ to 3′ in a genome of a prokaryotic cell. In some embodiments, jetA, and jetB, genes are positioned contiguously 5′ to 3′ in a genome of a prokaryotic cell. In some embodiments, jetA, jetB, and jetC genes are positioned sequentially 5′ to 3′ in a genome of a prokaryotic cell. In some embodiments, jetA, jetB, and jetC genes are positioned contiguously 5′ to 3′ in a genome of a prokaryotic cell. In some embodiments, jetA, jetB, jetC, and jetD genes are positioned sequentially 5′ to 3′ in a genome of a prokaryotic cell. In some embodiments, jetA, jetB, jetC, and jetD genes are positioned contiguously 5′ to 3′ in a genome of a prokaryotic cell. In some embodiments, jetD, jetA, jetB, and jetC genes are positioned sequentially 5′ to 3′ in a genome of a prokaryotic cell. In some embodiments, jetD, jetD, jetB, and jetC genes are positioned contiguously 5′ to 3′ in a genome of a prokaryotic cell.

In some embodiments, a JetA polypeptide is about 488 amino acids long (median gene size).

In some embodiments, the JetA polypeptide comprises the amino acid sequence having at least 80% homology to a sequence selected from the group consisting of the polypeptides referenced in Table 11, rows 2-3174, columns J and K. In some embodiments, the JetA polypeptide comprises the amino acid sequence having at least 80% homology to similar domain regions within a sequence selected from the group consisting of the polypeptides referenced in Table 11, rows 2-3174, columns J and K. In some embodiments, the JetA polypeptide comprises a homolog of a polypeptide having the amino acid sequences set forth in the polypeptides referenced in Table 11, rows 2-3174, columns J and K. In some embodiments, a homolog of a JetA polypeptide comprises a member of the MukF-like family. In some embodiments, the JetA polypeptide comprises the amino acid sequence having at least 80% identity to a sequence selected from the group consisting of the polypeptides referenced in Table 11, rows 2-3174, columns J and K. In some embodiments, the JetA polypeptide comprises the amino acid sequence having at least 80% identity to similar domain regions within a sequence selected from the group consisting of the polypeptides referenced in Table 11, rows 2-3174, columns J and K. In some embodiments, the JetA polypeptide comprises a homolog of a polypeptide having the amino acid sequences set forth in the polypeptides referenced in Table 11, rows 2-3174, columns J and K.

In some embodiments, the JetA polypeptide comprises the amino acid sequence having at least 80% identity to a sequence selected from the group consisting of the polypeptides referenced in Table 11, rows 2-3174, columns J and K. In some embodiments, the JetA polypeptide comprises the amino acid sequence having at least 80% identity within similar domain regions within a sequence selected from the group consisting of the polypeptides referenced in Table 11, rows 2-3174, columns J and K.

In some embodiments, an JetA polypeptide comprises a MukF-like domain. In some embodiments, an JetA polypeptide comprises a MukF-like activity.

In some embodiments, the JetA polypeptide comprises an amino acid sequence having at least 80%, at least 85%, at least 90%, or at least 95% homology to a sequence selected from the group consisting of the polypeptides referenced in Table 11, rows 2-3174, column J and K. In some embodiments, the JetA polypeptide comprises the amino acid sequence having at least 80%, 85%, 90%, 95% homology within similar domain regions within a sequence selected from the group consisting of the polypeptides referenced in Table 11, rows 2-3174, columns J and K. In some embodiments, the JetA polypeptide comprises an amino acid sequence having at least 80%, at least 85%, at least 90%, or at least 95% identity to a sequence selected from the group consisting of the polypeptides referenced in Table 11, rows 2-3174, columns J and K. In some embodiments, the JetA polypeptide comprises the amino acid sequence having at least 80%, 85%, 90%, 95% identity within similar domain regions within a sequence selected from the group consisting of the polypeptides referenced in Table 11, rows 2-3174, columns J and K. In some embodiments, the JetA polypeptide comprises an amino acid sequence selected from the group consisting of the polypeptides referenced in Table 11, rows 2-3174, columns J and K.

In some embodiments, the JetA polypeptide comprises an amino acid sequence encoded by a nucleic acid sequence having at least 80%, at least 85%, at least 90%, or at least 95% homology to a sequence selected from the group consisting of the polynucleotides referenced in Table 11, rows 2-3174, columns J and L. In some embodiments, the JetA polypeptide comprises the amino acid sequence encoded by a nucleic acid sequence having at least 80%, 85%, 90%, 95% homology within similar domain regions within a sequence selected from the group consisting of the polynucleotides referenced in Table 11, rows 2-3174, columns J. and L In some embodiments, the JetA polypeptide comprises an amino acid sequence encoded by a nucleic acid sequence having at least 80%, at least 85%, at least 90%, or at least 95% identity to a sequence selected from the group consisting of the polynucleotides referenced in Table 11, rows 2-3174, columns J and L. In some embodiments, the JetA polypeptide comprises the amino acid sequence encoded by a nucleic acid sequence having at least 80%, 85%, 90%, 95% homology within similar domain regions within a sequence selected from the group consisting of the polynucleotides referenced in Table 11, rows 2-3174, columns J and L. In some embodiments, the JetA polypeptide comprises an amino acid sequence encoded from a nucleic acid sequence selected from the group consisting of the polynucleotides referenced in Table 11, rows 2-3174, columns J and L.

In some embodiments, the nucleic acid sequence of a jetA gene comprises a nucleic acid sequence having at least 80%, at least 85%, at least 90%, or at least 95% homology to a sequence selected from the group consisting of the polynucleotides referenced in Table 11, rows 2-3174, columns J and L. In some embodiments, the jetA gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 11, rows 2-3174, columns J and L. In some embodiments, the nucleic acid sequence of a jetA gene comprises a nucleic acid sequence having at least 80%, at least 85%, at least 90%, or at least 95% identity to a sequence selected from the group consisting of the polynucleotides referenced in Table 11, rows 2-3174, columns J and L. In some embodiments, the jetA gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 11, rows 2-3174, columns J and L. In some embodiments, the nucleic acid sequence of a jetA gene comprises a nucleic acid sequence selected from the group consisting of the polynucleotides referenced in Table 11, rows 2-3174, columns J and L.

In some embodiments, the term “JetB” refers to the polynucleotide or expression product e.g., the polypeptide encoded by the jetB gene, comprising variants jetB, jetBⁱⁱ and jetBⁱⁱⁱ, associated with Defense Systems Xa, Xb, and Xc, respectively. In some embodiments, the terms “jetB”, “jetBⁱⁱ” and “jetBⁱⁱⁱ”, may be used interchangeably. In some embodiments, the term “JetB” refers to a JetB polypeptide or a JetBⁱⁱ polypeptide or a JetBⁱⁱⁱ polypeptide (FIG. 7B). In some embodiments, the terms JetB, JetBⁱⁱ, and JetBⁱⁱⁱ may be used interchangeably.

In some embodiments, the product of the jetB gene comprises a pfam13835 domain. In some embodiments, the product of the jetB gene comprises a DUF4194 domain. In some embodiments, the product of the jetB gene comprises a pfam09661 domain. In some embodiments, the product of the jetB gene comprises a DUF2398 domain. In some embodiments, the product of the jetB gene comprises a pfam13835 domain and a DUF4194 domain. In some embodiments, the product of the jetB gene comprises a pfam09661 domain and a DUF2398 domain. In some embodiments, the product of the jetB gene comprises a pfam13835 domain, or a DUF4194 domain, or pfam09661 domain, or a DUF2398 domain, or any combination thereof. In some embodiments, the product of the jetB gene comprises a pfam13835 domain, a DUF4194 domain, a pfam09661 domain, and a DUF2398 domain. In some embodiments, the jetB gene encodes a member of MukE-like family.

In some embodiments, JetB polypeptide is encoded by a gene positioned within 5-60 genes of a gene encoding a JetA, a JetC, and or a JetD polypeptide in a genome of a prokaryotic cell. In some embodiments, JetB polypeptide is encoded by a gene positioned within 5 genes upstream (5′) or downstream (3′) to a gene encoding a JetA, JetC, and or a JetD polypeptide in a genome of a prokaryotic cell.

In some embodiments, JetB and JetC are encoded by genes positioned sequentially 5′ to 3′ in a genome of a prokaryotic cell. In some embodiments JetB and JetC are encoded by genes positioned contiguously 5′ to 3′ in a genome of a prokaryotic cell. In some embodiments, JetB and JetD are encoded by genes positioned sequentially 5′ to 3′ in a genome of a prokaryotic cell. In some embodiments JetB and JetD are encoded by genes positioned contiguously 5′ to 3′ in a genome of a prokaryotic cell. In some embodiments, JetB and JetA are encoded by genes positioned sequentially 5′ to 3′ in a genome of a prokaryotic cell. In some embodiments JetB and JetA are encoded by genes positioned contiguously 5′ to 3′ in a genome of a prokaryotic cell. In some embodiments, JetB, JetC, and JetD are encoded by genes positioned sequentially 5′ to 3′ in a genome of a prokaryotic cell. In some embodiments, JetB, JetC, and JetD are encoded by genes positioned contiguously 5′ to 3′ in a genome of a prokaryotic cell.

In some embodiments, JetB polypeptide is encoded by a gene positioned within 5 genes upstream or downstream to a gene encoding jetA, jetC, and or jetD, in a genome of a prokaryotic cell. In some embodiments, JetB polypeptide is encoded by a gene positioned within 5 genes upstream or downstream to a gene encoding jetA in a genome of a prokaryotic cell. In some embodiments, JetB polypeptide is encoded by a gene positioned within 5 genes upstream or downstream to a gene encoding jetD in a genome of a prokaryotic cell. In some embodiments, JetB polypeptide is encoded by a gene positioned within 5 genes upstream or downstream to a gene encoding jetC in a genome of a prokaryotic cell.

In some embodiments, a JetB polypeptide is about 247 amino acids long (median gene size).

In some embodiments, the JetB polypeptide comprises an amino acid sequence having at least 80% homology to a sequence selected from the group consisting of the polypeptides referenced in Table 11, rows 2-3174, columns N and O. In some embodiments, the JetB polypeptide comprises an amino acid sequence having at least 80% homology to similar domain regions within a sequence selected from the group consisting of the polypeptides referenced in Table 11, rows 2-3174, columns N and O. In some embodiments, a homolog of the JetB polypeptide comprises a MukE-like family member. In some embodiments, the JetB polypeptide comprises an amino acid sequence having at least 80% identity to a sequence selected from the group consisting of the polypeptides referenced in Table 11, rows 2-3174, columns N and O. In some embodiments, the JetB polypeptide comprises an amino acid sequence having at least 80% identity to similar domain regions within a sequence selected from the group consisting of the polypeptides referenced in Table 11, rows 2-3174, columns N and O. In some embodiments, a JetB homolog comprises an amino acid sequence having at least 80% identity to a sequence selected from the group consisting of the polypeptides referenced in Table 11, rows 2-3174, columns N and O.

In some embodiments, a JetB polypeptide homolog comprises a MukE-like domain. In some embodiments, a JetB polypeptide homolog comprises a MukE-like activity.

In some embodiments, the JetB polypeptide comprises the amino acid sequence having at least 80% identity to a sequence selected from the group consisting of the polypeptides referenced in Table 11, rows 2-3174, columns N and O. In some embodiments, the JetB polypeptide comprises the amino acid sequence having at least 80% identity within similar domain regions within a sequence selected from the group consisting of the polypeptides referenced in Table 11, rows 2-3174, columns N and O.

In some embodiments, the JetB polypeptide comprises an amino acid sequence having at least 80%, at least 85%, at least 90%, or at least 95% homology to a sequence selected from the group consisting of the polypeptides referenced in Table 11, rows 2-3174, columns N and O. In some embodiments, the JetB polypeptide comprises the amino acid sequence having at least 80%, 85%, 90%, 95% homology within similar domain regions within a sequence selected from the group consisting of the polypeptides referenced in Table 11, rows 2-3174, columns N and O. In some embodiments, the JetB polypeptide comprises an amino acid sequence having at least 80%, at least 85%, at least 90%, or at least 95% identity to a sequence selected from the group consisting of the polypeptides referenced in Table 11, rows 2-3174, columns N and O. In some embodiments, the JetB polypeptide comprises the amino acid sequence having at least 80%, 85%, 90%, 95% identity within similar domain regions within a sequence selected from the group consisting of the polypeptides referenced in Table 11, rows 2-3174, columns N and O. In some embodiments, the JetB polypeptide comprises an amino acid sequence selected from the group consisting of the polypeptides referenced in Table 11, rows 2-3174, columns N and O.

In some embodiments, the JetB polypeptide comprises an amino acid sequence encoded by a nucleic acid sequence having at least 80%, at least 85%, at least 90%, or at least 95% homology to a sequence selected from the group consisting of the polynucleotides referenced in Table 11, rows 2-3174, columns N and P. In some embodiments, the JetB polypeptide comprises the amino acid sequence encoded by a nucleic acid sequence having at least 80%, 85%, 90%, 95% homology within similar domain regions within a sequence selected from the group consisting of the polynucleotides referenced in Table 11, rows 2-3174, columns N and P. In some embodiments, the JetB polypeptide comprises an amino acid sequence encoded by a nucleic acid sequence having at least 80%, at least 85%, at least 90%, or at least 95% identity to a sequence selected from the group consisting of the polynucleotides referenced in Table 11, rows 2-3174, columns N and P. In some embodiments, the JetB polypeptide comprises the amino acid sequence encoded by a nucleic acid sequence having at least 80%, 85%, 90%, 95% homology within similar domain regions within a sequence selected from the group consisting of the polynucleotides referenced in Table 11, rows 2-3174, columns N and P. In some embodiments, the JetB polypeptide comprises an amino acid sequence encoded from a nucleic acid sequence selected from the group consisting of the polynucleotides referenced in Table 11, rows 2-3174, columns N and P.

In some embodiments, the nucleic acid sequence of a jetB gene comprises a nucleic acid sequence having at least 80%, at least 85%, at least 90%, or at least 95% homology to a sequence selected from the group consisting of the polynucleotides referenced in Table 11, rows 2-3174, columns N and P. In some embodiments, the jetB gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 11, rows 2-3174, columns N and P. In some embodiments, the nucleic acid sequence of a jetB gene comprises a nucleic acid sequence having at least 80%, at least 85%, at least 90%, or at least 95% identity to a sequence selected from the group consisting of the polynucleotides referenced in Table 11, rows 2-3174, columns N and P. In some embodiments, the jetB gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 11, rows 2-3174, columns N and P. In some embodiments, the nucleic acid sequence of a jetB gene comprises a nucleic acid sequence selected from the group consisting of the polynucleotides referenced in Table 11, rows 2-3174, columns N and P.

In some embodiments, the term “JetC” refers to the polynucleotide or expression product e.g., the polypeptide encoded by the jetC gene, comprising variants jetC, jetCⁱⁱ and jetCⁱⁱⁱ, associated with Defense Systems Xa, Xb, and Xc, respectively. In some embodiments, the terms “jetC”, “jetCⁱⁱ” and “jetCⁱⁱⁱ”, may be used interchangeably. In some embodiments, the term “JetC” refers to a JetC polypeptide or a JetCⁱⁱ polypeptide or a JetCⁱⁱⁱ polypeptide (FIG. 7B). In some embodiments, the terms JetC, JetCⁱⁱ, and JetCⁱⁱⁱ may be used interchangeably.

In some embodiments, the product of a jetC gene comprises a pfam13555 domain. In some embodiments, the product of a jetC gene comprises a pfam13558 domain. In some embodiments, the product of a jetC gene comprises a COG4913 domain. In some embodiments, the product of a jetC gene comprises a COG1196 domain. In some embodiments, the product of a jetC gene comprises a pfam1555 domain or a pfam13558 domain or a COG4913 domain, or a COG1196 domain, or a combination thereof. In some embodiments, the product of a jetC gene comprises a pfam1555 domain and a pfam13558 domain. In some embodiments, the product of a jetC gene comprises a pfam1555 domain, a pfam13558 domain, and a COG4913 domain. In some embodiments, the product of a jetC gene comprises a pfam1555 domain and a COG4913 domain. In some embodiments, the product of a jetC gene comprises a pfam13558 domain and a COG4913 domain. In some embodiments, the product of a jetC gene comprises a MukB-like family member. MukB appears to be functionally related to the structural maintenance of chromosomes (SMC) proteins. MukB is also known as the E. coli condesin, a structural maintenance of chromosomes (SMC)-like protein, which forms a complex with MukE and the kleisin MukF. MukB is known to be able to mediate knotting of a DNA ring, an intramolecular reaction. In some embodiments, the product of a jetC gene comprises an ATP binding domain. In some embodiments, the product of a jetC gene comprises an exonuclease SbcC domain. In some embodiments, the product of a jetC gene comprises a MukB-like family member, or an ATP binding domain or an exonuclease SbcC domain, or a combination thereof. In some embodiments, the product of a jetC gene comprises a MukB-like family member and an ATP binding domain. In some embodiments, the product of a jetC gene comprises a MukB-like family member and an exonuclease SbcC domain. In some embodiments, the product of a jetC gene comprises an ATP binding domain and an exonuclease SbcC domain. In some embodiments, the product of a jetC gene comprises a MukB-like family member, an ATP binding domain and an exonuclease SbcC domain.

In some embodiments, JetC polypeptide is encoded by a gene positioned within 5-60 genes of a gene encoding JetA, and/or JetB and/or JetD in a genome of a prokaryotic cell. In some embodiments, JetC polypeptide is encoded by a gene positioned within 5 genes upstream (5′) or downstream (3′) to a gene encoding JetA, and/or JetB and/or JetD in a genome of a prokaryotic cell. In some embodiments, JetC polypeptide is encoded by a gene positioned within 5 genes upstream and downstream to a gene encoding JetA, and/or JetB and/or JetD in a genome of a prokaryotic cell.

In some embodiments, JetC and JetD are encoded by genes positioned sequentially 5′ to 3′ in a genome of a prokaryotic cell. In some embodiments, JetC and JetD are encoded by genes positioned contiguously 5′ to 3′ in a genome of a prokaryotic cell.

In some embodiments, jetC and jetD genes are positioned sequentially 5′ to 3′ in a genome of a prokaryotic cell. In some embodiments, jetC and jetD genes are positioned contiguously 5′ to 3′ in a genome of a prokaryotic cell.

In some embodiments, a JetC polypeptide is about 1476 amino acids long (median gene size).

In some embodiments, the JetC polypeptide comprises an amino acid sequence having at least 80% homology to a sequence selected from the group consisting of the polypeptides referenced in Table 11, rows 2-3174, columns R and S. In some embodiments, the JetC polypeptide comprises an amino acid sequence having at least 80% identity to a sequence selected from the group consisting of the polypeptides referenced in Table 11, rows 2-3174, columns R and S. In some embodiments, the JetC polypeptide comprises an amino acid sequence having at least 80% homology to similar domain regions within a sequence selected from the group consisting of the polypeptides referenced in Table 11, rows 2-3174, columns R and S. In some embodiments, the JetC polypeptide comprises an amino acid sequence having at least 80% identity to similar domain regions a sequence selected from the group consisting of the polypeptides referenced in Table 11, rows 2-3174, columns R and S. In some embodiments, an JetC homolog comprises an amino acid sequence having at least 80% homology to a sequence selected from the group consisting of the polypeptides referenced in Table 11, rows 2-3174, columns R and S.

In some embodiments, an JetC polypeptide homolog comprises an ATP binding domain. In some embodiments, an JetC polypeptide homolog comprises an exonuclease SbcC activity. In some embodiments, an JetC polypeptide homolog comprises an MukB like activity.

In some embodiments, the JetC polypeptide comprises an amino acid sequence having at least 80%, at least 85%, at least 90%, or at least 95% homology to a sequence selected from the group consisting of SEQ ID NOs: In some embodiments, the JetC polypeptide comprises an amino acid sequence having at least 80%, at least 85%, at least 90%, or at least 95% homology to similar domain regions within a sequence selected from the group consisting of the polypeptides referenced in Table 11, rows 2-3174, columns R and S. In some embodiments, a JetC homolog comprises an ATP binding domain. In some embodiments, a JetC homolog comprises an exonuclease SbcC activity. In some embodiments, a JetC homolog comprises a member of the MukB-like family. In some embodiments, a JetC homolog comprises an ATP binding domain, or an exonuclease SbcC activity, or a member of the MukB-like family, or any combination thereof. In some embodiments, a JetC homolog comprises an ATP binding domain and an exonuclease SbcC activity. In some embodiments, a JetC homolog comprises an ATP binding domain and a member of the MukB-like family. In some embodiments, a JetC homolog comprises an exonuclease SbcC activity and a member of the MukB-like family. In some embodiments, a JetC homolog comprises an ATP binding domain, and an exonuclease SbcC activity, and a member of the MukB-like family.

In some embodiments, the JetC polypeptide comprises an amino acid sequence having at least 80%, at least 85%, at least 90%, or at least 95% identity to a sequence selected from the group consisting of the polypeptides referenced in Table 11, rows 2-3174, columns R and S. In some embodiments, the JetC polypeptide comprises an amino acid sequence having at least 80%, at least 85%, at least 90%, or at least 95% identity to similar domain regions within a sequence selected from the group consisting of the polypeptides referenced in Table 11, rows 2-3174, columns R and S. In some embodiments, the JetC polypeptide comprises an amino acid sequence selected from the group consisting of the polypeptides referenced in Table 11, rows 2-3174, columns R and S.

In some embodiments, the JetC polypeptide comprises an amino acid sequence encoded by a nucleic acid sequence having at least 80%, at least 85%, at least 90%, or at least 95% homology to a sequence selected from the group consisting of the polynucleotides referenced in Table 11, rows 2-3174, columns R and T. In some embodiments, the JetC polypeptide comprises the amino acid sequence encoded by a nucleic acid sequence having at least 80%, 85%, 90%, 95% homology within similar domain regions within a sequence selected from the group consisting of the polynucleotides referenced in Table 11, rows 2-3174, columns R and T. In some embodiments, the JetC polypeptide comprises an amino acid sequence encoded by a nucleic acid sequence having at least 80%, at least 85%, at least 90%, or at least 95% identity to a sequence selected from the group consisting of the polynucleotides referenced in Table 11, rows 2-3174, columns R and T. In some embodiments, the JetC polypeptide comprises the amino acid sequence encoded by a nucleic acid sequence having at least 80%, 85%, 90%, 95% homology within similar domain regions within a sequence selected from the group consisting of the polynucleotides referenced in Table 11, rows 2-3174, columns R and T. In some embodiments, the JetC polypeptide comprises an amino acid sequence encoded from a nucleic acid sequence selected from the group consisting of the polynucleotides referenced in Table 11, rows 2-3174, columns R and T.

In some embodiments, the nucleic acid sequence of a jetC gene comprises a nucleic acid sequence having at least 80%, at least 85%, at least 90%, or at least 95% homology to a sequence selected from the group consisting of the polynucleotides referenced in Table 11, rows 2-3174, column N. In some embodiments, the jetC gene comprises a nucleic acid sequence having at least 80%, 85%, 90%, 95% homology within encoded similar domain regions in comparison with nucleotide sequence encoding these domain within a sequence selected from the group consisting of the polynucleotides referenced in Table 11, rows 2-3174, columns R and T. In some embodiments, the nucleic acid sequence of a jetC gene comprises a nucleic acid sequence having at least 80%, at least 85%, at least 90%, or at least 95% identity to a sequence selected from the group consisting of the polynucleotides referenced in Table 11, rows 2-3174, columns R and T. In some embodiments, the jetC gene comprises a nucleic acid sequence having at least 80%, 85%, 90%, 95% identity within encoded similar domain regions in comparison with nucleotide sequence encoding these domain within a sequence selected from the group consisting of the polynucleotides referenced in Table 11, rows 2-3174, columns R and T. In some embodiments, the nucleic acid sequence of a jetC gene comprises a nucleic acid sequence selected from the group consisting of the polynucleotides referenced in Table 11, rows 2-3174, columns R and T.

In some embodiments, the jetC gene homolog encodes a polypeptide comprising an ATP binding domain. In some embodiments, a jetC gene homolog encodes a polypeptide comprising an exonuclease SbcC activity. In some embodiments, a jetC gene homolog encodes a polypeptide comprising a member of the MukB-like family. In some embodiments, a JetC homolog jetC gene homolog encodes a polypeptide comprising ATP binding domain, or an exonuclease SbcC activity, or a member of the MukB-like family, or any combination thereof. In some embodiments, a jetC gene homolog encodes a polypeptide comprising an ATP binding domain and an exonuclease SbcC activity. In some embodiments, a jetC gene homolog encodes a polypeptide comprising an ATP binding domain and a member of the MukB-like family. In some embodiments, a jetC gene homolog encodes a polypeptide comprising an exonuclease SbcC activity and a member of the MukB-like family. In some embodiments, a jetC gene homolog encodes a polypeptide comprising an ATP binding domain, and an exonuclease SbcC activity, and a member of the MukB-like family.

In some embodiments, the term “JetD” refers to the polynucleotide or expression product e.g., the polypeptide encoded by the jetD gene, comprising variants jetD, jetDⁱⁱ and jetDⁱⁱⁱ, associated with Defense Systems Xa, Xb, and Xc, respectively. In some embodiments, the terms “jetD”, “jetDⁱⁱ” and “jetDⁱⁱⁱ”, may be used interchangeably. In some embodiments, the term “JetD” refers to a JetD polypeptide or a JetDⁱⁱ polypeptide or a JetDⁱⁱⁱ polypeptide (FIG. 7B). In some embodiments, the terms JetD, JetDⁱⁱ, and JetDⁱⁱⁱ may be used interchangeably.

In some embodiments, the product of the jetD gene comprises a pfam11795 domain. In some embodiments, the product of the jetD gene comprises a DUF3322 domain. In some embodiments, the product of the jetD gene comprises a pfam09983 domain. In some embodiments, the product of the jetD gene comprises a DUF2220 domain. In some embodiments, the product of the jetD gene comprises a pfam11796 domain. In some embodiments, the product of the jetD gene comprises a DUF3323 domain. In some embodiments, the product of the jetD gene comprises a pfam09664 domain. In some embodiments, the product of the jetD gene comprises a DUF2399 domain. In some embodiments, the product of the jetD gene comprises a COG4924 domain. In some embodiments, the product of the jetD gene comprises a pfam11795 domain, or a DUF3322 domain, or a pfam09983 domain, or a DUF2220 domain, or a pfam11796 domain, or a DUF3323 domain, or a pfam09664 domain, or DUF2399 domain, or a COG4924 domain, or any combination thereof. In some embodiments, the product of the jetD gene comprises a pfam11795 domain and a DUF3322 domain. In some embodiments, the product of the jetD gene comprises a pfam09983 and a DUF2220 domain. In some embodiments, the product of the jetD gene comprises a pfam11796 domain and a DUF3323 domain. In some embodiments, the product of the jetD gene comprises a pfam09664 domain and DUF2399 domain. In some embodiments, the product of the jetD gene comprises a pfam11796 domain and a DUF3323 domain, and a pfam09664 domain, and a DUF2399 domain. In some embodiments, the product of the jetD gene comprises a pfam11795 domain and a DUF3322 domain, and a pfam09983 domain, and a DUF2220 domain. In some embodiments, the product of the jetD gene comprises a pfam11795 domain and a DUF3322 domain, and a pfam09983 domain, and a DUF2220 domain, and a COG4924 domain.

In some embodiments, a JetD polypeptide comprises a pfam11795 domain. In some embodiments, a JetD polypeptide comprises a DUF3322 domain. In some embodiments, a JetD polypeptide comprises a pfam09983 domain. In some embodiments, a JetD polypeptide comprises a DUF2220 domain. In some embodiments, a JetD polypeptide comprises a pfam11796 domain. In some embodiments, a JetD polypeptide comprises a DUF3323 domain. In some embodiments, a JetD polypeptide comprises a pfam09664 domain. In some embodiments, a JetD polypeptide comprises a DUF2399 domain. In some embodiments, a JetD polypeptide comprises a COG4924 domain. In some embodiments, a JetD polypeptide comprises a pfam11795 domain, or a DUF3322 domain, or a pfam09983 domain, or a DUF2220 domain, or a pfam11796 domain, or a DUF3323 domain, or a pfam09664 domain, or DUF2399 domain, or a COG4924 domain, or any combination thereof. In some embodiments, a JetD polypeptide comprises a pfam11795 domain and a DUF3322 domain. In some embodiments, a JetD polypeptide comprises a pfam09983 and a DUF2220 domain. In some embodiments, a JetD polypeptide comprises a pfam11796 domain and a DUF3323 domain. In some embodiments, a JetD polypeptide comprises a pfam09664 domain and DUF2399 domain. In some embodiments, a JetD polypeptide comprises a pfam11796 domain and a DUF3323 domain, and a pfam09664 domain, and a DUF2399 domain. In some embodiments, a JetD polypeptide comprises a pfam11795 domain and a DUF3322 domain, and a pfam09983 domain, and a DUF2220 domain. In some embodiments, a JetD polypeptide comprises a pfam11795 domain and a DUF3322 domain, and a pfam09983 domain, and a DUF2220 domain, and a COG4924 domain.

In some embodiments, a JetD polypeptide is encoded by a gene positioned within 5-60 genes of a gene encoding JetA, and/or JetB and/or JetC in a genome of a prokaryotic cell. In some embodiments, a JetD polypeptide is encoded by a gene positioned within 5 genes upstream (5′) or downstream (3′) to a gene encoding JetA, and/or JetB and/or JetC in a genome of a prokaryotic cell. In some embodiments, JetD polypeptide is encoded by a gene positioned within 5 genes upstream or downstream to a gene encoding JetA, and/or JetB and JetC in a genome of a prokaryotic cell.

In some embodiments, a JetD polypeptide is about 347 amino acids long (median gene size).

In some embodiments, the JetD polypeptide comprises an amino acid sequence having at least 80% homology to a sequence selected from the group consisting of the polypeptides referenced in Table 11, rows 2-3174, columns V and W. In some embodiments, the JetD polypeptide comprises an amino acid sequence having at least 80% homology to similar domain regions within a sequence selected from the group consisting of the polypeptides referenced in Table 11, rows 2-3174, columns V and W. In some embodiments, a JetD polypeptide homologue comprises a topoisomerase IV/VI activity. In some embodiments, the JetD polypeptide comprises an amino acid sequence having at least 80% identity to a sequence selected from the group consisting of the polypeptides referenced in Table 11, rows 2-3174, columns V and W. In some embodiments, the JetD polypeptide comprises an amino acid sequence having at least 80% identity to similar domain regions within a sequence selected from the group consisting of the polypeptides referenced in Table 11, rows 2-3174, columns V and W. In some embodiments, an JetD homolog comprises an amino acid sequence having at least 80% identity to a sequence selected from the group consisting of the polypeptides referenced in Table 11, rows 2-3174, columns V and W.

In some embodiments, a JetD polypeptide homolog comprises a topoisomerase IV/VI domain. In some embodiments, a JetD polypeptide homolog comprises a topoisomerase IV/VI structural domain. In some embodiments, a JetD polypeptide homolog comprises a topoisomerase IV/VI activity.

In some embodiments, the JetD polypeptide comprises an amino acid sequence having at least 80%, at least 85%, at least 90%, at least 95% homology to a sequence selected from the group consisting of the polypeptides referenced in Table 11, rows 2-3174, columns V and W. In some embodiments, the JetD polypeptide comprises an amino acid sequence having at least 80%, at least 85%, at least 90%, at least 95% identity to a sequence selected from the group consisting of the polypeptides referenced in Table 11, rows 2-3174, columns V and W. In some embodiments, the JetD polypeptide comprises an amino acid sequence selected from the group consisting of the polypeptides referenced in Table 11, rows 2-3174, columns V and W.

In some embodiments, the JetD polypeptide comprises an amino acid sequence encoded from a nucleic acid sequence having at least 80%, at least 85%, at least 90%, or at least 95% homology to a sequence selected from the group consisting of the polynucleotides referenced in Table 11, rows 2-3174, columns V and X. In some embodiments, the JetD polypeptide comprises an amino acid sequence encoded from a nucleic acid sequence having at least 80%, at least 85%, at least 90%, or at least 95% identity to a sequence selected from the group consisting of the polynucleotides referenced in Table 11, rows 2-3174, columns V and X. In some embodiments, the JetD polypeptide comprises an amino acid sequence encoded from a nucleic acid sequence selected from the group consisting of the polynucleotides referenced in Table 11, rows 2-3174, columns V and X.

In some embodiments, the jetD gene comprises a nucleic acid sequence having at least 80%, at least 85%, at least 90%, or at least 95% homology to a sequence selected from the group consisting of the polynucleotides referenced in Table 11, rows 2-3174, columns V and X. In some embodiments, a mksG gene homolog encodes a polypeptide comprising a topoisomerase IV/VI activity. In some embodiments, the jetD gene comprises a nucleic acid sequence having at least 80%, at least 85%, at least 90%, or at least 95% identity to a sequence selected from the group consisting of the polynucleotides referenced in Table 11, rows 2-3174, columns V and X. In some embodiments, the nucleic acid sequence of the jetD gene is selected from the group consisting of the polynucleotides referenced in Table 11, rows 2-3174, columns V and X.

A skilled artisan would appreciate that the terms a “functional portion of a Defense System X component” or “functional fragment of Defense System X component” or “functional portion of a Wadjet defense system component” or “functional fragment of Wadjet defense system component” refers to a functional portion of a Wadjet polynucleotide or polypeptide, as disclosed herein, which expression is sufficient to elicit an anti-plasmid transformation efficiency activity alone or in combination with the at least one of the other Wadjet polynucleotides or polypeptides disclosed herein or functional portions thereof.

The terms “JetA”, “JetB”, “JetC”, “JetD”, “JetAⁱⁱ”, “JetBⁱⁱ”, “JetCⁱⁱ”, “JetDⁱⁱ”, jetAⁱⁱⁱ”, “jetBⁱⁱⁱ”, “jetCⁱⁱⁱ” and “jetDⁱⁱⁱ’ also refer to functional homologs, which exhibit the desired activity (i.e., conferring decreased efficiency of plasmid transformation). Such homologs can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide sequences referenced in Table 11 rows 2-3174 columns J and K, N and O, R and S and V and W, respectively, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polynucleotide sequences referenced in Table 11 rows 2-3174 columns J and L, N and P, R and T and V and X. In some embodiments, such homologs comprise at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% homology or identity within similar domain regions of the polypeptide sequences referenced in Table 11 rows 2-3174 columns J and K, N and O, R and S and V and W, or 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or homology within nucleotide sequences encoding similar domain regions to the polynucleotide sequences referenced in Table 11 rows 2-3174 columns J and L, N and P, R and T and V and X.

Table 11 presents embodiments of components of Defense System X (Xa, Xb, and Xc) that may be found in a diverse array of bacteria and archaea genomes (referenced in Table 18). Table 11 presents embodiments of nucleic acid sequences encoding gene cassettes of Defense System X (Xa, Xb, and Xc; referenced in Table 18).

Defense System Components, Nucleic Acid Sequences Thereof, and Amino Acid Sequences Thereof

Defense System components, nucleic acid sequences thereof, amino acid sequences thereof, and gene cassette nucleotide sequences thereof are presented below in Table 18.

TABLE 18
Defense Systems: Components and Gene Cassettes-
polypeptide and nucleotide sequences thereof

| | Com- | | |
|---|---|---|---|
| Defense System | ponent | Composition | Table References |
| Defense System Ia | ZorA | polypeptides | Table 8; Rows 2-1174; |
| ZORYA Type I | | | Column J “ZorA |
| | | | protein_accession” and |
| | | | Column K “PRT SEQ ID |
| | | | NO:” |
| Defense System Ia | ZorB | polypeptides | Table 8; Rows 2-1174; |
| ZORYA Type I | | | Column N “ZorB |
| | | | protein_accession” and |
| | | | Column O “PRT SEQ ID |
| | | | NO:” |
| Defense System Ia | ZorC | polypeptides | Table 8; Rows 2-1174; |
| ZORYA Type I | | | Column R “ZorC |
| | | | protein_accession” and |
| | | | Column S “PRT SEQ ID |
| | | | NO:” |
| Defense System Ia | ZorD | polypeptides | Table 8; Rows 2-1174; |
| ZORYA Type I | | | Column V “ZorD |
| | | | protein_accession” and |
| | | | Column W “PRT SEQ ID |
| | | | NO:” |
| Defense System Ia | zorA | poly- | Table 8; Rows 2-1174; |
| ZORYA Type I | | nucleotides | Column J “ZorA |
| | | | protein_accession” and |
| | | | Column L “DNA SEQ ID |
| | | | NO:” |
| Defense System Ia | zorB | poly- | Table 8; Rows 2-1174; |
| ZORYA Type I | | nucleotides | ColumnN “ZorB |
| | | | protein_accession” and |
| | | | Column P “DNA SEQ ID |
| | | | NO:” |
| Defense System Ia | zorC | poly- | Table 8; Rows 2-1174; |
| ZORYA Type I | | nucleotides | ColumnR “ZorC |
| | | | protein_accession” and |
| | | | Column T “DNA SEQ ID |
| | | | NO:” |
| Defense System Ia | zorD | poly- | Table 8; Rows 2-1174; |
| ZORYA Type I | | nucleotides | ColumnV “ZorD |
| | | | protein_accession” and |
| | | | Column X “DNA SEQ ID |
| | | | NO:” |
| Defense System Ia | Gene | poly- | Table 8; Rows 2-1174; |
| ZORYA Type I | Cassette | nucleotides | Column G |
| | | | “genomic_accession” with |
| | | | Start in Column AD |
| | | | “Genomic Start Point” and |
| | | | End in Column AE |
| | | | “Genomic End Point” |
| Defense System Ib | ZorA | polypeptides | Table 8; Rows 1175-1830; |
| ZORYA Type II | | | Column J “ZorA |
| | | | protein_accession” and |
| | | | Column K “PRT SEQ ID |
| | | | NO:” |
| Defense System Ib | ZorB | polypeptides | Table 8; Rows 1175-1830; |
| ZORYA Type I | | | ColumnN “ZorB |
| | | | protein_accession” and |
| | | | Column O “PRT SEQ ID |
| | | | NO:” |
| Defense System Ib | ZorE | polypeptides | Table 8; Rows 1175-1830; |
| ZORYA Type I | | | Column Z “ZorE |
| | | | protein_accession” and |
| | | | Column AA “PRT SEQ ID |
| | | | NO:” |
| Defense System Ib | zorA | poly- | Table 8; Rows 1175-1830; |
| ZORYA Type I | | nucleotides | Column J “ZorA |
| | | | protein_accession” and |
| | | | Column L “DNA SEQ ID |
| | | | NO:” |
| Defense System Ib | zorB | poly- | Table 8; Rows 1175-1830; |
| ZORYA Type I | | nucleotides | Column N “ZorB |
| | | | protein_accession” and |
| | | | Column P “DNA SEQ ID |
| | | | NO:” |
| Defense System Ib | zorE | poly- | Table 8; Rows 1175-1830; |
| ZORYA Type I | | nucleotides | ColumnZ “ZorE |
| | | | protein_accession” and |
| | | | Column AB “DNA SEQ ID |
| | | | NO:” |
| Defense System Ib | Gene | poly- | Table 8; Rows 1175-1830; |
| ZORYA Type I | Cassette | nucleotides | Column G |
| | | | “genomic_accession” with |
| | | | Start in Column AD |
| | | | “Genomic Start Point” and |
| | | | End in Column AE |
| | | | “Genomic End Point” |
| Defense System II | ThsA | polypeptides | Table 9; Rows 2-2100; |
| Thoeris | | | Column I “ThsA |
| | | | protein_accession” and |
| | | | Column J “PRT SEQ ID |
| | | | NO:” |
| Defense System II | ThsB | polypeptides | Table 9; Rows 2-2100; |
| Thoeris | | | Column M “ThsBⁱ |
| | | | protein_accession” and |
| | | | Column N “PRT SEQ ID |
| | | | NO:”, Column Q “ThsBⁱⁱ |
| | | | protein_accession” and |
| | | | Column R “PRT SEQ ID |
| | | | NO:”, Column U “ThsBⁱⁱⁱ |
| | | | protein_accession” and |
| | | | Column V “PRT SEQ ID |
| | | | NO:”, Column Y “ThsB^{iv} |
| | | | protein_accession” and |
| | | | Column Z “PRT SEQ ID |
| | | | NO:”, and Column AC |
| | | | “ThsB^{v} |
| | | | protein_accession” and |
| | | | Column AD “PRT SEQ ID |
| | | | NO:” |
| Defense System II | thsA | poly- | Table 9; Rows 2-2100; |
| Thoeris | | nucleotides | Column I “ThsA |
| | | | protein_accession” and |
| | | | Column K “DNA SEQ ID |
| | | | NO:” |
| Defense System II | thsB | poly- | Table 9; Rows 2-2100; |
| Thoeris | | nucleotides | Column M “ThsBⁱ |
| | | | protein_accession” and |
| | | | Column O “DNA SEQ ID |
| | | | NO:”, Column Q “ThsBⁱⁱ |
| | | | protein_accession” and |
| | | | Column S “DNA SEQ ID |
| | | | NO:”, Column U “ThsBⁱⁱⁱ |
| | | | protein_accession” and |
| | | | Column W “DNA SEQ ID |
| | | | NO:”, Column Y “ThsB^{iv} |
| | | | protein_accession” and |
| | | | Column AA “DNA SEQ ID |
| | | | NO:”, and Column AC |
| | | | “ThsB^{v} |
| | | | protein_accession” and |
| | | | Column AE “DNA SEQ ID |
| | | | NO:” |
| Defense System II | Gene | poly- | Table 9; Rows 2-2100; |
| Thoeris | Cassette | nucleotides | Column G |
| | | | “genomic_accession” with |
| | | | Start in Column AG |
| | | | “Genomic Start Point” and |
| | | | End in Column AH |
| | | | “Genomic End Point” |
| Defense System | DruA | polypeptides | Table 10; Rows 2-123; |
| IIIa Druantia | | | Column J “DruA |
| Type I | | | protein_accession” and |
| | | | Column K “PRT SEQ ID |
| | | | NO:” |
| Defense System | DruB | polypeptides | Table 10; Rows 2-123; |
| IIIa Druantia | | | Column N “DruB |
| Type I | | | protein_accession” and |
| | | | Column O “PRT SEQ ID |
| | | | NO:” |
| Defense System | DruC | polypeptides | Table 10; Rows 2-123; |
| IIIa Druantia | | | Column R “DruC |
| Type I | | | protein_accession” and |
| | | | Column S “PRT SEQ ID |
| | | | NO:” |
| Defense System | DruD | polypeptides | Table 10; Rows 2-123; |
| IIIa Druantia | | | Column V “DruD |
| Type I | | | protein_accession” and |
| | | | Column W “PRT SEQ ID |
| | | | NO:” |
| Defense System | DruE | polypeptides | Table 10; Rows 2-123; |
| IIIa Druantia | | | Column Z “DruE |
| Type I | | | protein_accession” and |
| | | | Column AA “PRT SEQ ID |
| | | | NO:” |
| Defense System | druA | poly- | Table 10; Rows 2-123; |
| IIIa Druantia | | nucleotides | Column J “DruA |
| Type I | | | protein_accession” and |
| | | | Column L “DNA SEQ ID |
| | | | NO:” |
| Defense System | druB | poly- | Table 10; Rows 2-123; |
| IIIa Druantia | | nucleotides | Column N “DruB |
| Type I | | | protein_accession” and |
| | | | Column P “DNA SEQ ID |
| | | | NO:” |
| Defense System | druC | poly- | Table 10; Rows 2-123; |
| IIIa Druantia | | nucleotides | Column R “DruC |
| Type I | | | protein_accession” and |
| | | | Column T “DNA SEQ ID |
| | | | NO:” |
| Defense System | druD | poly- | Table 10; Rows 2-123; |
| IIIa Druantia | | nucleotides | Column V “DruD |
| Type I | | | protein_accession” and |
| | | | Column X “DNA SEQ ID |
| | | | NO:” |
| Defense System | druE | poly- | Table 10; Rows 2-123; |
| IIIa Druantia | | nucleotides | Column Z “DruE |
| Type I | | | protein_accession” and |
| | | | Column AB “DNA SEQ ID |
| | | | NO:” |
| Defense System | Gene | poly- | Table 10; Rows 2-123; |
| IIIa Druantia | Cassette | nucleotides | Column G |
| Type I | | | “genomic_accession” with |
| | | | Start in Column AT |
| | | | “Genomic Start Point” and |
| | | | End in Column AU |
| | | | “Genomic End Point” |
| Defense System | DruM | polypeptides | Table 10; Rows 124-295; |
| IIIb Druantia | | | Column AD “DruM |
| Type II | | | protein_accession” and |
| | | | Column AE “PRT SEQ ID |
| | | | NO:” |
| Defense System | DruF | polypeptides | Table 10; Rows 124-295; |
| IIIb Druantia | | | Column AH “DruF |
| Type II | | | protein_accession” and |
| | | | Column AI “PRT SEQ ID |
| | | | NO:” |
| Defense System | DruG | polypeptides | Table 10; Rows 124-295; |
| IIIb Druantia | | | Column AL “DruG |
| Type II | | | protein_accession” and |
| | | | Column AM “PRT SEQ ID |
| | | | NO:” |
| Defense System | DruE | polypeptides | Table 10; Rows 124-295; |
| IIIb Druantia | | | Column Z “DruE |
| Type II | | | protein_accession” and |
| | | | Column AA “PRT SEQ ID |
| | | | NO:” |
| Defense System | druM | poly- | Table 10; Rows 124-295; |
| IIIb Druantia | | nucleotides | Column AD “DruM |
| Type II | | | protein_accession” and |
| | | | Column AF “DNA SEQ ID |
| | | | NO:” |
| Defense System | druF | poly- | Table 10; Rows 124-295; |
| IIIb Druantia | | nucleotides | Column AH “DruF |
| Type II | | | protein_accession” and |
| | | | Column AJ “DNA SEQ ID |
| | | | NO:” |
| Defense System | druG | poly- | Table 10; Rows 124-295; |
| IIIb Druantia | | nucleotides | Column AL “DruG |
| Type II | | | protein_accession” and |
| | | | Column AN “DNA SEQ ID |
| | | | NO:” |
| Defense System | druE | poly- | Table 10; Rows 124-295; |
| IIIb Druantia | | nucleotides | Column Z “DruE |
| Type II | | | protein_accession” and |
| | | | Column AB “DNA SEQ ID |
| | | | NO:” |
| Defense System | Gene | poly- | Table 10; Rows 124-295; |
| IIIb Druantia | Cassette | nucleotides | Column G |
| Type II | | | “genomic_accession” with |
| | | | Start in Column AT |
| | | | “Genomic Start Point” and |
| | | | End in Column AU |
| | | | “Genomic End Point” |
| Defense System | DruH | polypeptides | Table 10; Rows 296-1343; |
| IIIc Druantia | | | Column AP “DruH |
| Type III | | | protein_accession” and |
| | | | Column AQ “PRT SEQ ID |
| | | | NO:” |
| Defense System | DruE | polypeptides | Table 10; Rows 296-1343; |
| IIIc Druantia | | | Column Z “DruE |
| Type III | | | protein_accession” and |
| | | | Column AA “PRT SEQ ID |
| | | | NO:” |
| Defense System | druH | poly- | Table 10; Rows 296-1343; |
| IIIc Druantia | | nucleotides | Column AP “DruH |
| Type III | | | protein_accession” and |
| | | | Column AR “DNA SEQ ID |
| | | | NO:” |
| Defense System | druE | poly- | Table 10; Rows 296-1343; |
| IIIc Druantia | | nucleotides | Column Z “DruE |
| Type III | | | protein_accession” and |
| | | | Column AB “DNA SEQ ID |
| | | | NO:” |
| Defense System | Gene | poly- | Table 10; Rows 296-1343; |
| IIIc Druantia | Cassette | nucleotides | Column G |
| Type III | | | “genomic_accession” with |
| | | | Start in Column AT |
| | | | “Genomic Start Point” and |
| | | | End in Column AU |
| | | | “Genomic End Point” |
| Defense System IV | HamA | polypeptides | Table 12; Rows 2-1782; |
| Hachiman | | | Column H “HamA |
| | | | protein_accession” and |
| | | | Column I “PRT SEQ ID |
| | | | NO:” |
| Defense System | HamB | polypeptides | Table 12; Rows 2-1782; |
| IV Hachiman | | | Column L “HamB |
| | | | protein_accession” and |
| | | | Column M “PRT SEQ ID |
| | | | NO:” |
| Defense System | hamA | poly- | Table 12; Rows 2-1782; |
| IV Hachiman | | nucleotides | Column H “HamA |
| | | | protein_accession” and |
| | | | Column J “DNA SEQ ID |
| | | | NO:” |
| Defense System | hamB | poly- | Table 12; Rows 2-1782; |
| IV Hachiman | | nucleotides | Column L “HamB |
| | | | protein_accession” and |
| | | | Column N “DNA SEQ ID |
| | | | NO:” |
| Defense System | Gene | poly- | Table 12; Rows 2-1782; |
| IV Hachiman | Cassette | nucleotides | Column G |
| | | | “genomic_accession” with |
| | | | Start in Column P |
| | | | “Genomic Start Point” |
| | | | and End in Column Q |
| | | | “Genomic End Point” |
| Defense System V | SduA | polypeptides | Table 13; Rows 2-1247; |
| Shedu | | | Column H “SduA |
| | | | protein_accession” and |
| | | | Column I “PRT SEQ ID |
| | | | NO:” |
| Defense System V | sduA | poly- | Table 13; Rows 2-1247; |
| Shedu | | nucleotides | Column H “SduA |
| | | | protein_accession” and |
| | | | Column J “DNA SEQ ID |
| | | | NO:” |
| Defense System V | Gene | poly- | Table 13; Rows 2-1782; |
| Shedu | Cassette | nucleotides | Column G |
| | | | “genomic_accession” with |
| | | | Start in Column L |
| | | | “Genomic Start Point” |
| | | | and End in Column M |
| | | | “Genomic End Point” |
| Defense System | GajA | polypeptides | Table 14; Rows 2-4599; |
| VI Gabija | | | Column H “GajA |
| | | | protein_accession” and |
| | | | Column I “PRT SEQ ID |
| | | | NO:” |
| Defense System | GajB | polypeptides | Table 14; Rows 2-4599; |
| VI Gabija | | | Column L “GajB |
| | | | protein_accession” and |
| | | | Column M “PRT SEQ ID |
| | | | NO:” |
| Defense System | gajA | poly- | Table 14; Rows 2-4599; |
| VI Gabija | | nucleotides | Column H “GajA |
| | | | protein_accession” and |
| | | | Column J “DNA SEQ ID |
| | | | NO:” |
| Defense System | gajB | poly- | Table 14; Rows 2-4599; |
| VI Gabija | | nucleotides | Column L “GajB |
| | | | protein_accession” and |
| | | | Column N “DNA SEQ ID |
| | | | NO:” |
| Defense System | Gene | poly- | Table 14; Rows 2-4599; |
| VI Gabija | Cassette | nucleotides | Column G |
| | | | “genomic_accession” with |
| | | | Start in Column P |
| | | | “Genomic Start Point” |
| | | | and End in Column Q |
| | | | “Genomic End Point” |
| Defense System | PtuA | polypeptides | Table 15; Rows 2-2507; |
| VII Septu | | | Column H “PtuA |
| | | | protein_accession” and |
| | | | Column I “PRT SEQ ID |
| | | | NO:” |
| Defense System | PtuB | polypeptides | Table 15; Rows 2-2507; |
| VII Septu | | | Column L “PtuB |
| | | | protein_accession” and |
| | | | Column M “PRT SEQ ID |
| | | | NO:” |
| Defense System | ptuA | poly- | Table 15; Rows 2-2507; |
| VII Septu | | nucleotides | Column H “PtuA |
| | | | protein_accession” and |
| | | | Column J “DNA SEQ ID |
| | | | NO:” |
| Defense System | ptuB | poly- | Table 15; Rows 2-2507; |
| VII Septu | | nucleotides | Column L “PtuB |
| | | | protein_accession” and |
| | | | Column N “DNA SEQ ID |
| | | | NO:” |
| Defense System | Gene | poly- | Table 15; Rows 2-2507; |
| VII Septu | Cassette | nucleotides | Column G |
| | | | “genomic_accession” with |
| | | | Start in Column P |
| | | | “Genomic Start Point” |
| | | | and End in Column Q |
| | | | “Genomic End Point” |
| Defense System | LmuA | polypeptides | Table 16; Rows 2-698; |
| VIII Lamussa | | | Column H “LmuA |
| | | | protein_accession” and |
| | | | Column I “PRT SEQ ID |
| | | | NO:” |
| Defense System | LmuB | polypeptides | Table 16; Rows 2-698; |
| VIII Lamussa | | | Column L “LmuB |
| | | | protein_accession” and |
| | | | Column M “PRT SEQ ID |
| | | | NO:” |
| Defense System | lmuA | poly- | Table 16; Rows 2-698; |
| VIII Lamussa | | nucleotides | Column H “LmuA |
| | | | protein_accession” and |
| | | | Column J “DNA SEQ ID |
| | | | NO:” |
| Defense System | lmuB | poly- | Table 16; Rows 2-698; |
| VIII Lamussa | | nucleotides | ColumnL “LmuB |
| | | | protein_accession” and |
| | | | Column N “DNA SEQ ID |
| | | | NO:” |
| Defense System | Gene | poly- | Table 16; Rows 2-698; |
| VIII Lamussa | Cassette | nucleotides | Column G |
| | | | “genomic_accession” with |
| | | | Start in Column P |
| | | | “Genomic Start Point” |
| | | | and End in Column Q |
| | | | “Genomic End Point” |
| Defense System | KwaA | polypeptides | Table 17; Rows 2-935; |
| IX Kiwa | | | Column H “KwaA |
| | | | protein_accession” and |
| | | | Column I “PRT SEQ ID |
| | | | NO:” |
| Defense System | KwaB | polypeptides | Table 17; Rows 2-935; |
| IX Kiwa | | | Column L “KwaB |
| | | | protein_accession” and |
| | | | Column M “PRT SEQ ID |
| | | | NO:” |
| Defense System | kwaA | poly- | Table 17; Rows 2-935; |
| IX Kiwa | | nucleotides | Column H “KwaA |
| | | | protein_accession” and |
| | | | Column J “DNA SEQ ID |
| | | | NO:” |
| Defense System | kwaB | poly- | Table 17; Rows 2-935; |
| IX Kiwa | | nucleotides | Column L “KwaB |
| | | | protein_accession” and |
| | | | Column N “DNA SEQ ID |
| | | | NO:” |
| Defense System | Gene | poly- | Table 17; Rows 2-935; |
| IX Kiwa | Cassette | nucleotides | Column G |
| | | | “genomic_accession” with |
| | | | Start in Column P |
| | | | “Genomic Start Point” |
| | | | and End in Column Q |
| | | | “Genomic End Point” |
| Defense System | JetA | polypeptides | Table 11; Rows 2-2322; |
| Xa Wadjet type I | | | Column J “JetA |
| | | | protein_accession” and |
| | | | Column K “PRT SEQ ID |
| | | | NO:” |
| Defense System | JetB | polypeptides | Table 11; Rows 2-2322; |
| Xa Wadjet type I | | | Column N “JetB |
| | | | protein_accession” and |
| | | | Column O “PRT SEQ ID |
| | | | NO:” |
| Defense System | JetC | polypeptides | Table 11; Rows 2-2322; |
| Xa Wadjet type I | | | Column R “JetC |
| | | | protein_accession” and |
| | | | Column S “DNA SEQ ID |
| | | | NO:” |
| Defense System | JetD | polypeptides | Table 11; Rows 2-2322; |
| Xa Wadjet type I | | | Column V “JetD |
| | | | protein_accession” and |
| | | | Column W “PRT SEQ ID |
| | | | NO:” |
| Defense System | jetA | poly- | Table 11; Rows 2-2322; |
| Xa Wadjet type I | | nucleotides | Column J “JetA |
| | | | protein_accession” and |
| | | | Column L “DNA SEQ ID |
| | | | NO:” |
| Defense System | jetB | poly- | Table 11; Rows 2-2322; |
| Xa Wadjet type I | | nucleotides | Column N “JetB |
| | | | protein_accession” and |
| | | | Column P “DNA SEQ ID |
| | | | NO:” |
| Defense System | jetC | poly- | Table 11; Rows 2-2322; |
| Xa Wadjet type I | | nucleotides | Column R “JetC |
| | | | protein_accession” and |
| | | | Column T “DNA SEQ ID |
| | | | NO:” |
| Defense System | jetD | poly- | Table 11; Rows 2-2322; |
| Xa Wadjet type I | | nucleotides | Column V “JetD |
| | | | protein_accession” and |
| | | | Column X “DNA SEQ ID |
| | | | NO:” |
| Defense System | Gene | poly- | Table 11; Rows 2-2322; |
| Xa Wadjet type I | Cassette | nucleotides | Column G |
| | | | “genomic_accession” with |
| | | | Start in Column Z |
| | | | “Genomic Start Point” |
| | | | and End in Column AA |
| | | | “Genomic End Point” |
| Defense System | JetAⁱⁱ | polypeptides | Table 11; Rows 2323-2844; |
| Xb Wadjet type II | | | Column J “JetA |
| | | | protein_accession” and |
| | | | Column K “PRT SEQ ID |
| | | | NO:” |
| Defense System | JetBⁱⁱ | polypeptides | Table 11; Rows 2323-2844; |
| Xb Wadjet type II | | | Column N “JetB |
| | | | protein_accession” and |
| | | | Column O “DNA SEQ ID |
| | | | NO:” |
| Defense System | JetCⁱⁱ | polypeptides | Table 11; Rows 2323-2844; |
| Xb Wadjet type II | | | Column R “JetC |
| | | | protein_accession” and |
| | | | Column S “PRT SEQ ID |
| | | | NO:” |
| Defense System | JetDⁱⁱ | polypeptides | Table 11; Rows 2323-2844; |
| Xb Wadjet type II | | | Column V “JetD |
| | | | protein_accession” and |
| | | | Column W “PRT SEQ ID |
| | | | NO:” |
| Defense System | jetAⁱⁱ | poly- | Table 11; Rows 2323-2844; |
| Xb Wadjet type II | | nucleotides | Column J “JetA |
| | | | protein_accession” and |
| | | | Column L “DNA SEQ ID |
| | | | NO:” |
| Defense System | jetBⁱⁱ | poly- | Table 11; Rows 2323-2844; |
| Xb Wadjet type II | | nucleotides | Column N “JetB |
| | | | protein_accession” and |
| | | | Column P “DNA SEQ ID |
| | | | NO:” |
| Defense System | jetCⁱⁱ | poly- | Table 11; Rows 2323-2844; |
| Xb Wadjet type II | | nucleotides | Column R “JetC |
| | | | protein_accession” and |
| | | | Column T “DNA SEQ ID |
| | | | NO:” |
| Defense System | jetDⁱⁱ | poly- | Table 11; Rows 2323-2844; |
| Xb Wadjet type II | | nucleotides | Column V “JetD |
| | | | protein_accession” and |
| | | | Column X “DNA SEQ ID |
| | | | NO:” |
| Defense System | Gene | poly- | Table 11; Rows 2323-2844; |
| Xb Wadjet type II | Cassette | nucleotides | Column G |
| | | | “genomic_accession” with |
| | | | Start in Column Z |
| | | | “Genomic Start Point” |
| | | | and End in Column AA |
| | | | “Genomic End Point” |
| Defense System | JetAⁱⁱⁱ | polypeptides | Table 11; Rows 2845-3174; |
| Xc Wadjet type | | | Column J “JetA |
| III | | | protein_accession” and |
| | | | Column K “PRT SEQ ID |
| | | | NO:” |
| Defense System | JetBⁱⁱⁱ | polypeptides | Table 11; Rows 2845-3174; |
| Xc Wadjet type | | | Column N “JetB |
| III | | | protein_accession” and |
| | | | Column O “PRT SEQ ID |
| | | | NO:” |
| Defense System | JetCⁱⁱⁱ | polypeptides | Table 11; Rows 2845-3174; |
| Xc Wadjet type | | | Column R “JetC |
| III | | | protein_accession” and |
| | | | Column S “PRT SEQ ID |
| | | | NO:” |
| Defense System | JetDⁱⁱⁱ | polypeptides | Table 11; Rows 2845-3174; |
| Xc Wadjet type | | | Column V “JetD |
| III | | | protein_accession” and |
| | | | Column W “PRT SEQ ID |
| | | | NO:” |
| Defense System | jetAⁱⁱⁱ | poly- | Table 11; Rows 2845-3174; |
| Xc Wadjet type | | nucleotides | Column J “JetA |
| III | | | protein_accession” and |
| | | | Column L “DNA SEQ ID |
| | | | NO:” |
| Defense System | jetBⁱⁱⁱ | poly- | Table 11; Rows 2845-3174; |
| Xc Wadjet type | | nucleotides | Column N “JetB |
| III | | | protein_accession” and |
| | | | Column P “DNA SEQ ID |
| | | | NO:” |
| Defense System | jetCⁱⁱⁱ | poly- | Table 11; Rows 2845-3174; |
| Xc Wadjet type | | nucleotides | Column R “JetC |
| III | | | protein_accession” and |
| | | | Column T “DNA SEQ ID |
| | | | NO:” |
| Defense System | jetDⁱⁱⁱ | poly- | Table 11; Rows 2845-3174; |
| Xc Wadjet type | | nucleotides | Column V “JetD |
| III | | | protein_accession” and |
| | | | Column X “DNA SEQ ID |
| | | | NO:” |
| Defense System | Gene | poly- | Table 11; Rows 2845-3174; |
| Xc Wadjet type | Cassette | nucleotides | Column G |
| III | | | “genomic_accession” with |
| | | | Start in Column Z |
| | | | “Genomic Start Point” |
| | | | and End in Column AA |
| | | | “Genomic End Point” |

Table 18 lists embodiments of the components of each Defense System and embodiments gene cassettes encoding the Defense Systems. Provided are the Sequence Numbers comprising the polypeptide and polynucleotide sequences, or explicit Reference to the Accession numbers provided in Tables 8-17, respectively for each Defense System, from which the skilled artisan can access the polypeptide and polynucleotide sequences. While Table 18 highlights polypeptide components of defense systems and the genes sequences that encode them, in some embodiments, defense system components include non-coding RNA and/or regulatory nucleic acid sequences.

Using the Reference locations provided in Table 18 and the “protein accession” numbers provided in Tables 8-17, one can reach both the amino acid and the nucleotide sequences of a Defense System component. For example, to access the polypeptide (amino acid) sequence of a component, a skilled artisan would find the Defense System and component of interest in Table 18. Next, they would use the Reference information provided in Table 18 to identify the series of accession numbers that encompass the polypeptide sequence of the component. Following that, the skilled artisan would query the NCBI “Protein” database: https://www.ncbi.nlm.nih.gov/protein, and enter the accession number provided for a component, for example but not limited to “ABS66238.1” (Table 8, row 2, column J), which would send them to https://www.ncbi.nlm.nih.gov/protein/CEP95570.1, wherein the amino acid sequence of the component is disclosed on this page, as follows:

(SEQ ID NO: 1)
MTVLKGLGRCVLFWTITGIFGVLYWLLPVVDMSALWGGTARAFSGDFSTV
GTQPYAFALAFALFATAVGFASAFLILHVGPIGWRLRRLRLRIAGTGDME
GFARTYDATRALMERDRLISHAWKQFDTVLVHRPDERIIRSTARAHAFFN
LGMAREKYFGLKMMGALPGYFVGIGLLLTFAGLVLALNKAAMAVNSSDAA
GMQGATRELLQVATFKFATSIAGLGASIALSLLFRIYTILMEGFFIGFCE
TVEDRVRYLSAQQIAAESRDFAAEQTDQLKSMSADFFARMGQELTPGMSR
MFETALDKVMSPVTTSIDQAVHRLADSSQSGMSEMLTRFTDSVQNGAGAE
LRGLAETLRGMQGAMVGVQQGLTGSGEDFGRRMSDAAENLNRLVTDAGAR
LGEGTDQSRAVLMDAVTAMRETFEQANRKVDEGLGAAAGGASARLEEVMG
RVLGSLEAQVGGFRESLSGFQEKMAGQLDETRVRVSAAQAEATDAVAQAS
AQAARALQDGLADALVRINGEIERFVVAMRASEVTLAAQAKAMMDATDKS
RQVADAFSRTAQDVRAASVPLAQSGERIAGATEKLGEAAGRSVAVLDKSQ
EEARRLAQALTGHVSNLDQVWRSYSARFEAVDEALGKAFERLTRGTDEQQ
DRIATFVRDVDVTFKEAVDTLAGCIGGLKDNTEEMVEAVDDLKRALRVEA
AE.

Similarly, to access the nucleotide (nucleic acid) sequence of a component, a skilled artisan would find the Defense System and component of interest in Table 18. Next, they would use the Reference information provided in Table 18 to identify the series of accession numbers that encompass the nucleotide sequence of the component. Following that, the skilled artisan would query the NCBI “Identical Protein Groups” database https://www.ncbi.nlm.nih.gov/ipg, and enter the accession number provided for a component, for example but not limited to “ABS66238.1” (Table 8, row 2, column J), which would send them to https://www.ncbi.nlm.nih.gov/ipg/?term=ABS66238.1, wherein any of the links under “CDS Region in Nucleotide” leads to the nucleotide sequence:

### Origin

| (SEQ ID NO: 2) | |
|---|---|
| 1 | atgacggtct tgaagggttt ggggcgctgc gttctttttt ggacaatcat attcggcatc |
| 61 | ttcggcgtct tgtactggct ccttcctgtc gtcgacatga gcgcgctctg gggcgggacg |
| 121 | gcgcgcgcct tcagcggtga tttttccacc gtcggcaccc agccctatgc tttcgccctg |
| 181 | gccttcgccc tgttcgccac ggcggtcggc ttcgcctccg ctttcctcat cctgcatgtg |
| 241 | ggtccgatcg ggtggaggct gcggcgcctg cgattgcgta tcgcgggcac gggggacatg |
| 301 | gaaggcttcg ccagaaccta tgacgccacg cgcgcgctca tggagcgcga tcgactgatc |
| 361 | agccatgcgt ggaagcagtt cgacaccgtg ctcgttcatc ggccggatga acgcatcatc |
| 421 | cgcagcaccg cccgtgccca cgcctttttc aatctgggca tggcgcggga gaagtatttc |
| 481 | ggcctgaaga tgatgggcgc gctgccgggc tatttcgtcg gcatcggtct tctgctcacc |
| 541 | ttcgccggcc tcgtgctggc gctcaacaag gcggccatgg ccgtcaacag ctcggatgcc |
| 601 | gccggaatgc agggcgcgac gcgcgaactg cttcaggtgg cgaccttcaa gtttgccacc |
| 661 | tccatcgccg gccttggtgc ctccatcgcg ctctcgctgc tgttccgcat ctataccatc |
| 721 | ctgatggaag gcttcttcat cggcttctgc gagacggtgg aggaccgggt gcgctatctg |
| 781 | tcggcacagc agatcgcggc cgagagccgg gattttgccg ccgaacagac cgatcagctc |
| 841 | aagtcgatca acagcgccga cttcttcgcg cgcatggggc aggagctgac gccgggcatg |
| 901 | tcccgcatgt tcgagacggc gctcgacaag gtcatgtcgc cggtgaccac cagcatcgac |
| 961 | caggcggtcc ataggctcgc cgacagcagc cagagcggca tgtccgagat gctgacgcgc |
| 1021 | ttcaccgatt cggtgcagaa cggggccggg gccgagttgc gcgggctcgc cgagacgctc |
| 1081 | cggggcatgc agggtgccat ggtgggcgtg cagcaggggc tgaccgggtc cggcgaggat |
| 1141 | ttcggccggc gcatgtcgga tgccgcggaa aacctcaacc gcctcgtcac cgacgccggc |
| 1201 | gcacgcctgg gtgaaggcac ggaccagagc cgcgcggtcc tcatggatgc ggtcaccgcc |
| 1261 | atgcgggaga ccttcgagca agccaatcgc aaggtggacg agggtctcgg cgccgcggcg |
| 1321 | ggcggggctt cggcgcggct ggaggaggtc atggggcggg tgctcggcag cctcgaagcc |
| 1381 | caggtgggcg gctttcgcga gagcctgtcc ggcttccagg agaagatggc cggccagctc |
| 1441 | gatgagaccc gggtccgcgt ctccgccgcc caggcggagg cgaccgatgc cgtcgcccag |
| 1501 | gcctcggccc aggcggcgcg cgcgctccag gacgggcttg cggatgcgct cgtgcgcatc |
| 1561 | aacggcgaga tcgagcgttt cgtcgtcgcc atgcgggcca gcgaggtgac gctggcggcg |
| 1621 | caggccaagg cgatgatgga cgccaccgac aagtcgcgcc aggtggccga tgccttcagc |
| 1681 | cgcactgcgc aggatgtgcg cgcggctagc gtaccgctgg cccaatcggg cgagcgcatc |
| 1741 | gcgggggcga ccgagaagct cggcgaggcc gccggccggt cggtggcggt cctcgacaag |
| 1801 | agccaggagg aggcgcggcg gctggcccag gcgctgaccg gccatgtctc caacctcgat |
| 1861 | caggtctggc gcagctattc ggcccgcttc gaggcggtcg acgaggcgct gggcaaggcg |
| 1921 | ttcgagcgcc tgacgcgcgg aaccgacgag cagcaggacc ggatcgccac tttcgttcgc |
| 1981 | gacgtggacg tgaccttcaa ggaagcggtg gatacgctgg ccggctgcat cggcggcctg |
| 2041 | aaggacaata ccgaggagat ggtggaggcg gtggacgacc tgaaacgcgc gctgcgcgtc |
| 2101 | gaggccgcgg aatga. |

### Defense Systems & Combination of Defense Systems

Described above are non-limiting examples of defense systems, wherein each system comprises components that together make-up a defense system, wherein in some embodiments each component is encoded by a different gene. In some embodiments, the combination of components that make-up a Defense System, for example the components that make-up each of Defense Systems Ia-Xc disclosed above, may include components comprising nucleic acid sequences or amino acid sequences derived from different species (different donor species). In some embodiments, the combination of components that make-up a Defense System, for example the components that make-up each of Defense Systems Ia-Xc, may include homologs of the genes and/or polypeptides identified in donor species. In some embodiments, the combination of components that make-up a Defense System, for example the components that make-up each of Defense Systems Ia-Xc, differs from the combination of components found in donor species. In some embodiments, the components of a Defense System, for example the components that make-up each of Defense Systems Ia-Xc, comprises less than all the components found in donor species. In some embodiments, the components of a Defense System, for example the components that make-up each of Defense Systems Ia-Xc, comprises duplications of the components found in donor species, or multiple copies of one or more components found in a donor species. In some embodiments, the components of a Defense System, for example the components that make-up each of Defense Systems Ia-Xc, comprise functional portions of the components found in donor species, wherein a functional component comprise less than the full length of the components present in a donor species. In some embodiments, the order of components encoded on the nucleic acid sequence comprised within a construct comprising a Defense System, for example the order of the components the make-up each of Defense Systems Ia-Xc, differs from the order of the components found encoded on the nucleic acid sequences in donor species.

In some embodiments each Defense Systems Ia, Ib, II, III, IV, V, VI, VII, VIII, IX, Xa, Xb, and Xc comprising a nucleic acid construct comprising a nucleic acid sequence encoding the polypeptide components as disclosed herein, further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof.

In some embodiments, a construct further comprises a regulatory element operably linked to said construct comprising a cis-acting regulatory element for directing expression of said nucleic acid sequence. In some embodiment, the nucleic acid sequence of the regulatory element is from the same species as at least one component of the Defense System. In some embodiment, the nucleic acid sequence of the regulatory element is not from the same species as any of the components of the Defense System. In some embodiment, the nucleic acid sequence of the regulatory element is not from the donor species of the Defense System. In some embodiment, when a host cell comprises a Defense System, the nucleic acid sequence of the regulatory element is from the host species.

In some embodiments, cis-acting regulatory elements include those that direct constitutive expression of a nucleic acid sequence. In some embodiments, cis-acting regulatory elements comprise those that direct inducible expression of the nucleic acid sequence only under certain conditions.

According to some embodiments, the cis-acting regulatory element is heterologous to the genes encoding the components of a system. In some embodiments, the cis-acting regulatory element is heterologous to Defense System Ia genes zorA, zorB, zorC, and zorD. In some embodiments, the cis-acting regulatory element is heterologous to Defense System Ib genes zorA, zorB, and zorE. In some embodiments, the cis-acting regulatory element is heterologous to Defense System II genes thsA and thsB. In some embodiments, the cis-acting regulatory element is heterologous to Defense System IIIa genes druA, druB, druC, drub, and druE. In some embodiments, the cis-acting regulatory element is heterologous to Defense System IIIb genes druM, druF, druG, and druE. In some embodiments, the cis-acting regulatory element is heterologous to Defense System IIIc genes druH and druE. In some embodiments, the cis-acting regulatory element is heterologous to Defense System IV genes hamA and hamB. In some embodiments, the cis-acting regulatory element is heterologous to Defense System V component sduA. In some embodiments, the cis-acting regulatory element is heterologous to Defense System VI genes gajA and gajB. In some embodiments, the cis-acting regulatory element is heterologous to Defense System VII genes ptuA and ptuB. In some embodiments, the cis-acting regulatory element is heterologous to Defense System VIII genes lmuA and lmuB. In some embodiments, the cis-acting regulatory element is heterologous to Defense System IX genes kwaA and kwaB. In some embodiments, the cis-acting regulatory element is heterologous to Defense System Xa genes jetA, jetB, jetC, and jetD. In some embodiments, the cis-acting regulatory element is heterologous to Defense System Xb genes jetAⁱⁱ, jetBⁱⁱ, jetCⁱⁱ, and jetDⁱⁱ. In some embodiments, the cis-acting regulatory element is heterologous to Defense System Xc genes jetAⁱⁱⁱ, jetBⁱⁱⁱ, jetCⁱⁱⁱ, and jetDⁱⁱⁱ.

According to some embodiments, the nucleic acid construct includes a promoter sequence for directing transcription of the nucleic acid sequence in a host cell in a constitutive or inducible manner. In some embodiments, a promoter is a constitutive promoter. In some embodiments, a promoter is an inducible or regulatable promoter.

Constitutive promoters suitable for use with some embodiments of the Defense System constructs disclosed herein are promoter sequences which are active under most environmental conditions and most types of cells such as the cytomegalovirus (CMV) and Rous sarcoma virus (RSV). Inducible promoters suitable for use with some embodiments of Defense System constructs disclosed herein, include for example but not limited to the tetracycline-inducible promoter (Zabala M, et al., Cancer Res. 2004, 64(8): 2799-804) or pathogen-inducible promoters. Such promoters include those from pathogenesis-related proteins (PR proteins), which are induced following infection by a pathogen.

According to some embodiments, the promoter is bacterial nucleic acid (e.g., expression) construct.

A skilled artisan would appreciate that bacterial promoter encompasses any DNA sequence capable of binding bacterial RNA polymerase and initiating the downstream (3′) transcription of a coding sequence into mRNA. A promoter can have a transcription initiation region, which is usually placed proximal to the 5′ end of the coding sequence. This transcription initiation region typically includes an RNA polymerase binding site and a transcription initiation site. A bacterial promoter can also have a second domain called an operator, which can overlap an adjacent RNA polymerase binding site at which RNA synthesis begins. The operator permits negative regulated (inducible) transcription, as a gene repressor protein can bind the operator and thereby inhibit transcription of a specific gene. Constitutive expression can occur in the absence of negative regulatory elements, such as the operator. In addition, positive regulation can be achieved by a gene activator protein binding sequence, which, if present is usually proximal (5′) to the RNA polymerase binding sequence.

A non-limiting example of a gene activator protein is the catabolite activator protein (CAP), which helps initiate transcription of the lac operon in Escherichia coli (Raibaud et al. (1984) Annu. Rev. Genet. 18:173). Regulated expression can therefore be either positive or negative, thereby either enhancing or reducing transcription. Other examples of positive and negative regulatory elements are well known in the art. Various promoters that can be included in the protein expression system include, but are not limited to, a T7/LacO hybrid promoter, a trp promoter, a T7 promoter, a lac promoter, and a bacteriophage lambda promoter. Any suitable promoter can be used with the Defense systems disclosed herein, including the native promoter or a heterologous promoter. Heterologous promoters can be constitutively active or inducible. A non-limiting example of a heterologous promoter is given in U.S. Pat. No. 6,242,194 to Kullen and Klaenhammer.

Sequences encoding metabolic pathway enzymes provide particularly useful promoter sequences. Examples include promoter sequences derived from sugar metabolizing enzymes, such as galactose, lactose (lac) (Chang et al. (1987) Nature 198:1056), and maltose. Additional examples include promoter sequences derived from biosynthetic enzymes such as tryptophan (trp) (Goeddel et al. (1980) Nucleic Acids Res. 8:4057; Yelverton et al. (1981) Nucleic Acids Res. 9:731; U.S. Pat. No. 4,738,921; EPO Publication Nos. 36,776 and 121,775). The beta-lactamase (bla) promoter system (Weissmann, (1981) “The Cloning of Interferon and Other Mistakes,” in Interferon 3 (ed. I. Gresser); bacteriophage lambda PL (Shimatake et al. (1981) Nature 292:128); the arabinose-inducible araB promoter (U.S. Pat. No. 5,028,530); and T5 (U.S. Pat. No. 4,689,406) promoter systems also provide useful promoter sequences. See also Balbas (2001) Mol. Biotech. 19:251-267, where E. coli expression systems are discussed.

In addition, synthetic promoters that do not occur in nature also function as bacterial promoters. For example, transcription activation sequences of one bacterial or phage promoter can be joined with the operon sequences of another bacterial or phage promoter, creating a synthetic hybrid promoter (U.S. Pat. No. 4,551,433). For example, the tac (Amann et al. (1983) Gene 25:167; de Boer et al. (1983) Proc. Natl. Acad. Sci. 80:21) and trc (Brosius et al. (1985) J. Biol. Chem. 260:3539-3541) promoters are hybrid trp-lac promoters comprised of both trp promoter and lac operon sequences that are regulated by the lac repressor. The tac promoter has the additional feature of being an inducible regulatory sequence. Thus, for example, expression of a coding sequence operably linked to the tac promoter can be induced in a cell culture by adding isopropyl-1-thio-β-D-galactoside (IPTG). Furthermore, bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The phage T7 RNA polymerase/promoter system is an example of a coupled promoter system (Studier et al. (1986) J. Mol. Biol. 189:113; Tabor et al. (1985) Proc. Natl. Acad. Sci. 82:1074). In addition, a hybrid promoter can also be comprised of a phage promoter and an E. coli operator region (EPO Publication No. 267,851).

The nucleic acid construct can additionally contain a nucleic acid sequence encoding the repressor (or inducer) for that promoter. For example, an inducible construct can regulate transcription from the Lac operator (LacO) by expressing the nucleotide sequence encoding the Lad repressor protein. Other examples include the use of the lexA gene to regulate expression of pRecA, and the use of trpO to regulate ptrp. Alleles of such genes that increase the extent of repression (e.g., lacIq) or that modify the manner of induction (e.g., lambda CI857, rendering lambda pL thermo-inducible, or lambda CI+, rendering lambda pL chemo-inducible) can be employed.

In the construction of the construct, in some embodiments, the promoter is positioned approximately the same distance from the heterologous transcription start site as it is from the transcription start site in its natural setting. As is known in the art, however, some variation in this distance can be accommodated without loss of promoter function.

According to some embodiments, the nucleic acid construct includes a promoter sequence for directing transcription of the nucleic acid sequence in the cell in a constitutive or inducible manner. In some embodiments, the expression of a Defense System disclosed herein can be transient or consistent, episomal or integrated into the chromosome of a host cell. According to some embodiments, the expression is on a transmissible genetic element.

The nucleic acid construct of some embodiments of a Defense System disclosed herein may further include additional sequences which render this construct suitable for replication and integration in prokaryotes, eukaryotes, or preferably both (e.g., shuttle vectors).

In some embodiments, the construct comprises a recombination element for integrating the polynucleotide into a genome of a cell transfected with the construct.

A skilled artisan would appreciate that the term “recombination element” encompasses a nucleic acid sequence that allows the integration of the polynucleotide in the genome of a cell (e.g. bacteria) transfected with the construct.

In some embodiments, a construct may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

In some embodiments, a construct further comprises a transmissible element for directing transfer of said nucleic acid sequence from one cell to another. In some embodiments, a Defense System Ia or the Defense System Ia component(s) is on a transmissible genetic element. In some embodiments, a Defense System Ib or the Defense System Ib component(s) is on a transmissible genetic element. In some embodiments, a Defense System IIIa or the Defense System IIIa component(s) is on a transmissible genetic element. In some embodiments, a Defense System IIIb or the Defense System IIIb component(s) is on a transmissible genetic element. In some embodiments, a Defense System IIIc or the Defense System IIIc component(s) is on a transmissible genetic element. In some embodiments, a Defense System IV or the Defense System IV component(s) is on a transmissible genetic element. In some embodiments, a Defense System V or the Defense System V component(s) is on a transmissible genetic element. In some embodiments, a Defense System VI or the Defense System VI component(s) is on a transmissible genetic element. In some embodiments, a Defense System VII or the Defense System VII component(s) is on a transmissible genetic element. In some embodiments, a Defense System VIII or the Defense System VIII component(s) is on a transmissible genetic element. In some embodiments, a Defense System IX or the Defense System IX component(s) is on a transmissible genetic element. In some embodiments, a Defense System Xa or the Defense System Xa component(s) is on a transmissible genetic element. In some embodiments, a Defense System Xb or the Defense System Xb component(s) is on a transmissible genetic element. In some embodiments, a Defense System Xc or the Defense System Xc component(s) is on a transmissible genetic element.

In some embodiments, a transmissible genetic element comprises the construct of a Defense System disclosed herein. In some embodiments, a transmissible genetic element comprises the construct of Defense System Ia, Ib, II, IIIa, IIIb, IIIc, IV, V, VI, VII, VIII, IX, Xa, Xb, or Xc. In some embodiments, a transmissible genetic element comprises the construct of Defense System Ia. In some embodiments, a transmissible genetic element comprises the construct of Defense System Ib. In some embodiments, a transmissible genetic element comprises the construct of Defense System II. In some embodiments, a transmissible genetic element comprises the construct of Defense System IIIa. In some embodiments, a transmissible genetic element comprises the construct of Defense System IIIb. In some embodiments, a transmissible genetic element comprises the construct of Defense System IIIc. In some embodiments, a transmissible genetic element comprises the construct of Defense System IV. In some embodiments, a transmissible genetic element comprises the construct of Defense System V. In some embodiments, a transmissible genetic element comprises the construct of Defense System VI. In some embodiments, a transmissible genetic element comprises the construct of Defense System VII. In some embodiments, a transmissible genetic element comprises the construct of Defense System VIII. In some embodiments, a transmissible genetic element comprises the construct of Defense System IX. In some embodiments, a transmissible genetic element comprises the construct of Defense System Xa. In some embodiments, a transmissible genetic element comprises the construct of Defense System Xb. In some embodiments, a transmissible genetic element comprises the construct of Defense System Xc.

A skilled artisan would appreciate that the term “transmissible element” or “transmissible genetic element”, which are interchangeably used, encompasses a polynucleotide that allows the transfer of the nucleic acid sequence from one cell to another, e.g. from one bacteria to another.

According to some embodiments, a transmissible genetic element comprises a conjugative genetic element or mobilizable genetic element. In some embodiments, a transmissible genetic element comprises a conjugative genetic element. In some embodiments, a transmissible genetic element comprises a mobilizable genetic element. The skilled artisan would appreciate that a “conjugative plasmid” encompasses a plasmid that is transferred from one cell (e.g. bacteria) to another during conjugation, and the term “mobilizable element” encompasses a transposon, which is a DNA sequence that can change its position within the genome.

In some embodiments, a nucleic acid construct disclosed herein, comprises an expression vector. In some embodiments, an “expression vector” or a “vector”, used interchangeably herein, comprises and expresses the Defense System components from a single or a plurality of constructs (i.e. construct). In some embodiments, expression comprises transient expression. In some embodiments, expression comprises constitutive expression. In some embodiments, expression is from an episomal nucleic acid sequence. In some embodiments, expression is from a nucleic acid sequence integrated into the chromosome of the cell. According to specific embodiments, the expression is on a transmissible genetic element.

According to some embodiments, a construct further comprises a recombination element for integrating the nucleic acid sequence into a genome of cell transfected with the construct. A skilled artisan would appreciate that the term “recombination element” encompasses a polynucleotide that allows the integration of the nucleic acid sequence in the genome of a cell (e.g. bacteria) transfected with the construct.

In addition, typical constructs may also, in certain embodiments, contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

According to some embodiment, the nucleic acid construct comprises a plurality of cloning sites for ligating a nucleic acid sequence of a Defense System, such that it is under transcriptional regulation of the regulatory elements.

Selectable marker genes that ensure maintenance of a construct in a host cell can also be included in the construct. In some embodiments, selectable markers include those which confer resistance to drugs such as ampicillin, chloramphenicol, erythromycin, kanamycin (neomycin), and tetracycline (Davies et al. (1978) Annu. Rev. Microbiol. 32:469). Selectable markers can also allow a cell to grow on minimal medium, or in the presence of toxic metabolite and can include biosynthetic genes, such as those in the histidine, tryptophan, and leucine biosynthetic pathways.

Other than containing the necessary elements for the transcription and translation of the inserted coding sequence, the expression construct of some embodiments of Defense system can also include sequences engineered to enhance stability, production, purification, yield or toxicity of the expressed polypeptide. Where appropriate, the nucleic acid sequences may be optimized for increased expression in the transformed organism. For example, the nucleic acid sequences can be synthesized using preferred codons for improved expression.

Various methods known within the art can be used to introduce the construct of some embodiments of a Defense System disclosed herein into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, natural or induced transformation, lipofection, electroporation and infection with recombinant viral vectors. In addition, see U.S. Pat. Nos. 5,464,764 and 5,487,992 for positive-negative selection methods.

Some methods of introducing a construct or constructs into bacterial cells include for example conventional transformation or transfection techniques, or by phage-mediated infection. A skilled artisan would appreciate that the terms “transformation”, “transduction”, “conjugation”, and “protoplast fusion” are intended to encompass a variety of art-recognized techniques for introducing foreign nucleic acid (e.g., DNA) into a cell, such as calcium chloride co-precipitation. A skilled artisan would appreciate that introduction of a construct or constructs into a bacterial cell, may in certain embodiments, result in expression of a polypeptide or polypeptides encoded by the construct or constructs.

Introduction of nucleic acids by phage infection offers several advantages over other methods such as transformation, since higher transfection efficiency can be obtained due to the infectious nature of phages. These methods are especially useful for rendering bacteria more sensitive to phage attack for antibiotics purposes as further described hereinbelow.

It will be appreciated that a Defense System polypeptide(s) can be introduced directly into the cell (e.g., bacterial cell) and not via recombinant expression to confer resistance. Thus, according to some embodiments, disclosed herein are isolated polypeptides of each of the Defense systems' components and functional fragments thereof as described herein. In some embodiments, a polypeptide component of a Defense System can be introduced directly into the cell (e.g., bacterial cell) and not via recombinant expression, to confer resistance. In some embodiments, at least two different polypeptide components of a Defense System can be introduced directly into the cell (e.g., bacterial cell) and not via recombinant expression, to confer resistance. In some embodiments, at least three different polypeptide components of a Defense System can be introduced directly into the cell (e.g., bacterial cell) and not via recombinant expression, to confer resistance. In some embodiments, at least four polypeptide components of a Defense System can be introduced directly into the cell (e.g., bacterial cell) and not via recombinant expression, to confer resistance. In some embodiments, at least five different polypeptide components of a Defense System can be introduced directly into the cell (e.g., bacterial cell) and not via recombinant expression, to confer resistance. In some embodiments, multiple polypeptide components of a Defense System can be introduced directly into the cell (e.g., bacterial cell) and not via recombinant expression, to confer resistance.

In some embodiments, a Defense System Ia polypeptides can be introduced directly into the cell (e.g., bacterial cell) and not via recombinant expression to confer resistance. In some embodiments, disclosed herein are isolated polypeptides of each of the Defense system Ia's components and functional fragments thereof as described herein. In some embodiments, a Defense System Ib polypeptides can be introduced directly into the cell (e.g., bacterial cell) and not via recombinant expression to confer resistance. In some embodiments, disclosed herein are isolated polypeptides of each of the Defense system Ib's components and functional fragments thereof as described herein. In some embodiments, Defense System II polypeptides can be introduced directly into the cell (e.g., bacterial cell) and not via recombinant expression to confer resistance. In some embodiments, disclosed herein are isolated polypeptides of each of the Defense system II's components and functional fragments thereof as described herein. In some embodiments, Defense System IIIa polypeptides can be introduced directly into the cell (e.g., bacterial cell) and not via recombinant expression to confer resistance. In some embodiments, disclosed herein are isolated polypeptides of each of the Defense system IIIa's components and functional fragments thereof as described herein. In some embodiments, Defense System IIIb polypeptides can be introduced directly into the cell (e.g., bacterial cell) and not via recombinant expression to confer resistance. In some embodiments, disclosed herein are isolated polypeptides of each of the Defense system IIIb's components and functional fragments thereof as described herein. In some embodiments, Defense System IIIc polypeptides can be introduced directly into the cell (e.g., bacterial cell) and not via recombinant expression to confer resistance. In some embodiments, disclosed herein are isolated polypeptides of each of the Defense system IIIc's components and functional fragments thereof as described herein. In some embodiments, Defense System IV polypeptides can be introduced directly into the cell (e.g., bacterial cell) and not via recombinant expression to confer resistance. In some embodiments, disclosed herein are isolated polypeptides of each of the Defense system IV components and functional fragments thereof as described herein. In some embodiments, Defense System V polypeptides can be introduced directly into the cell (e.g., bacterial cell) and not via recombinant expression to confer resistance. In some embodiments, disclosed herein are isolated polypeptides of each of the Defense System V components and functional fragments thereof as described herein. In some embodiments, Defense System VI polypeptides can be introduced directly into the cell (e.g., bacterial cell) and not via recombinant expression to confer resistance. In some embodiments, disclosed herein are isolated polypeptides of each of the Defense system VI's components and functional fragments thereof as described herein. In some embodiments, Defense System VII polypeptides can be introduced directly into the cell (e.g., bacterial cell) and not via recombinant expression to confer resistance. In some embodiments, disclosed herein are isolated polypeptides of each of the Defense system VII's components and functional fragments thereof as described herein. In some embodiments, Defense System VIII polypeptides can be introduced directly into the cell (e.g., bacterial cell) and not via recombinant expression to confer resistance. In some embodiments, disclosed herein are isolated polypeptides of each of the Defense system VIII's components and functional fragments thereof as described herein. In some embodiments, Defense System IX polypeptides can be introduced directly into the cell (e.g., bacterial cell) and not via recombinant expression to confer resistance. In some embodiments, disclosed herein are isolated polypeptides of each of the Defense system IX's components and functional fragments thereof as described herein. In some embodiments, a Defense System Xa polypeptides can be introduced directly into the cell (e.g., bacterial cell) and not via recombinant expression to confer resistance. In some embodiments, disclosed herein are isolated polypeptides of each of the Defense System Xa's components and functional fragments thereof as described herein. In some embodiments, a Defense System Xb polypeptides can be introduced directly into the cell (e.g., bacterial cell) and not via recombinant expression to confer resistance. In some embodiments, disclosed herein are isolated polypeptides of each of the Defense System Xb's components and functional fragments thereof as described herein. In some embodiments, a Defense System Xc polypeptides can be introduced directly into the cell (e.g., bacterial cell) and not via recombinant expression to confer resistance. In some embodiments, disclosed herein are isolated polypeptides of each of the Defense System Xc's components and functional fragments thereof as described herein.

In some embodiments, at least one Defense System component is on a transmissible genetic element. In some embodiments, more than one Defense System component, from the same Defense system, is on a transmissible genetic element. In some embodiments, two Defense System components, from the same Defense system, are on a transmissible genetic element. In some embodiments, three Defense System components, from the same Defense system, are on a transmissible genetic element. In some embodiments, four Defense System components, from the same Defense system, are on a transmissible genetic element. In some embodiments, five Defense System components, from the same Defense system, are on a transmissible genetic element. In some embodiments, all of the Defense System components from a single Defense system are on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, at least one Defense System Ia component is on a transmissible genetic element. In some embodiments, more than one Defense System Ia component, is on a transmissible genetic element. In some embodiments, two Defense System Ia components, are on a transmissible genetic element. In some embodiments, three Defense System Ia components, are on a transmissible genetic element. In some embodiments, four Defense System Ia components, are on a transmissible genetic element. In some embodiments, all of the Defense System Ia components are on a transmissible genetic element. In some embodiments, the nucleic acid construct of Defense System Ia is on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, at least one Defense System Ib component is on a transmissible genetic element. In some embodiments, more than one Defense System Ib component, is on a transmissible genetic element. In some embodiments, two Defense System Ib components, are on a transmissible genetic element. In some embodiments, three Defense System Ib components, are on a transmissible genetic element. In some embodiments, all of the Defense System Ib components are on a transmissible genetic element. In some embodiments, the nucleic acid construct of Defense System Ib is on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, at least one Defense System II component is on a transmissible genetic element. In some embodiments, more than one Defense System II component, is on a transmissible genetic element. In some embodiments, two Defense System II components, are on a transmissible genetic element. In some embodiments, all of the Defense System II components are on a transmissible genetic element. In some embodiments, the nucleic acid construct of Defense System II is on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, at least one Defense System IIIa component is on a transmissible genetic element. In some embodiments, more than one Defense System IIIa component, is on a transmissible genetic element. In some embodiments, two Defense System IIIa components, are on a transmissible genetic element. In some embodiments, three Defense System IIIa components, are on a transmissible genetic element. In some embodiments, four Defense System IIIa components, are on a transmissible genetic element. In some embodiments, five Defense System IIIa components, are on a transmissible genetic element. In some embodiments, all of the Defense System IIIa components are on a transmissible genetic element. In some embodiments, the nucleic acid construct of Defense System IIIa is on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, at least one Defense System IIIb component is on a transmissible genetic element. In some embodiments, more than one Defense System IIIb component, is on a transmissible genetic element. In some embodiments, two Defense System IIIb components, are on a transmissible genetic element. In some embodiments, three Defense System IIIb components, are on a transmissible genetic element. In some embodiments, four Defense System IIIb components, are on a transmissible genetic element. In some embodiments, five Defense System IIIb components, are on a transmissible genetic element. In some embodiments, all of the Defense System IIIb components are on a transmissible genetic element. In some embodiments, the nucleic acid construct of Defense System IIIb is on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, at least one Defense System IIIc component is on a transmissible genetic element. In some embodiments, more than one Defense System IIIc component, is on a transmissible genetic element. In some embodiments, two Defense System IIIc components, are on a transmissible genetic element. In some embodiments, three Defense System IIIc components, are on a transmissible genetic element. In some embodiments, four Defense System IIIc components, are on a transmissible genetic element. In some embodiments, five Defense System IIIc components, are on a transmissible genetic element. In some embodiments, all of the Defense System IIIc components are on a transmissible genetic element. In some embodiments, the nucleic acid construct of Defense System IIIc is on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, at least one Defense System IV component is on a transmissible genetic element. In some embodiments, more than one Defense System IV component, is on a transmissible genetic element. In some embodiments, two Defense System IV components, are on a transmissible genetic element. In some embodiments, all of the Defense System IV components are on a transmissible genetic element. In some embodiments, the nucleic acid construct of Defense System IV is on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, at least one Defense System V component is on a transmissible genetic element. In some embodiments, the nucleic acid construct of Defense System V is on a transmissible genetic element. In some embodiments, all of the Defense System V components are on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, at least one Defense System VI component is on a transmissible genetic element. In some embodiments, more than one Defense System VI component, is on a transmissible genetic element. In some embodiments, two Defense System VI components, are on a transmissible genetic element. In some embodiments, all of the Defense System VI components are on a transmissible genetic element. In some embodiments, the nucleic acid construct of Defense System VI is on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, at least one Defense System VII component is on a transmissible genetic element. In some embodiments, more than one Defense System VII component, is on a transmissible genetic element. In some embodiments, two Defense System VII components, are on a transmissible genetic element. In some embodiments, all of the Defense System VII components are on a transmissible genetic element. In some embodiments, the nucleic acid construct of Defense System VII is on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, at least one Defense System VIII component is on a transmissible genetic element. In some embodiments, more than one Defense System VIII component, is on a transmissible genetic element. In some embodiments, two Defense System VIII components, are on a transmissible genetic element. In some embodiments, all of the Defense System VIII components are on a transmissible genetic element. In some embodiments, the nucleic acid construct of Defense VIII is on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, at least one Defense System IX component is on a transmissible genetic element. In some embodiments, more than one Defense System IX component, is on a transmissible genetic element. In some embodiments, two Defense System IX components, are on a transmissible genetic element. In some embodiments, all of the Defense System IX components are on a transmissible genetic element. In some embodiments, the nucleic acid construct of Defense System IX is on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, at least one Defense System Xa component is on a transmissible genetic element. In some embodiments, more than one Defense System Xa component, is on a transmissible genetic element. In some embodiments, two Defense System Xa components, are on a transmissible genetic element. In some embodiments, three Defense System Xa components, are on a transmissible genetic element. In some embodiments, four Defense System Xa components, are on a transmissible genetic element. In some embodiments, all of the Defense System Xa components are on a transmissible genetic element. In some embodiments, the nucleic acid construct of Defense System Xa is on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, at least one Defense System Xb component is on a transmissible genetic element. In some embodiments, more than one Defense System Xb component, is on a transmissible genetic element. In some embodiments, two Defense System Xb components, are on a transmissible genetic element. In some embodiments, three Defense System Xb components, are on a transmissible genetic element. In some embodiments, four Defense System Xb components, are on a transmissible genetic element. In some embodiments, all of the Defense System Xb components are on a transmissible genetic element. In some embodiments, the nucleic acid construct of Defense System Xb is on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, at least one Defense System Xc component is on a transmissible genetic element. In some embodiments, more than one Defense System Xc component, is on a transmissible genetic element. In some embodiments, two Defense System Xc components, are on a transmissible genetic element. In some embodiments, three Defense System Xc components, are on a transmissible genetic element. In some embodiments, four Defense System Xc components, are on a transmissible genetic element. In some embodiments, all of the Defense System Xc components are on a transmissible genetic element. In some embodiments, the nucleic acid construct of Defense System Xc is on a transmissible genetic element. In some embodiments, the components are on the same transmissible genetic element. In some embodiments, the components are on different transmissible elements. In some embodiments, the order of the components on a transmissible genetic element is as found in a donor species. In some embodiments, the order of the components on a transmissible genetic element differs from the order found in a donor species.

In some embodiments, a construct disclosed herein, further comprises an element providing episomal maintenance of said construct within a cell transfected with said construct.

In some embodiments, a Defense System construct comprising a Defense System Ia, Ib, II, IIIa, IIIb, IIIc, IV, V, VI, VII, VIII, IX, Xa, Xb, or Xc further comprises a regulatory element operably linked to said construct comprising a cis-acting regulatory element for directing expression of said nucleic acid sequence. In some embodiments, a Defense System construct comprising a Defense System Ia, Ib, II, IIIa, IIIb, IIIc, IV, V, VI, VII, VIII, IX, Xa, Xb, or Xc further comprises a transmissible element for directing transfer of said nucleic acid sequence from one cell to another. In some embodiments, a Defense System construct comprising a Defense System Ia, Ib, II, IIIa, IIIb, IIIc, IV, V, VI, VII, VIII, IX, Xa, Xb, or Xc further comprises a recombination element for integrating said nucleic acid sequence into a genome of a cell transfected with said construct. In some embodiments, a Defense System construct comprising a Defense System Ia, Ib, II, IIIa, IIIb, IIIc, IV, V, VI, VII, VIII, IX, Xa, Xb, or Xc further comprises an element providing episomal maintenance of said construct within a cell transfected with said construct. In some embodiments, a Defense System construct comprising a Defense System Ia, Ib, II, IIIa, IIIb, IIIc, IV, V, VI, VII, VIII, IX, Xa, Xb, or Xc further comprises any combination of a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof.

In some embodiments, a defense system described herein comprises a combination of Defense Systems. In some embodiments, a defense system described herein comprises a combination of Defense Systems (Ia)-(Xc). In some embodiments, the combination of Defense Systems, for example but not limited to the combination of Defense Systems (Ia)-(Xc), provides advantageous properties including (1) resistance to a broader range of phages or (2) resistance to at least one phage and reduced transformation efficiency of plasmids or (3) both resistance to a broader range of phages and reduced transformation efficiency.

A skilled artisan would appreciate that the terms “anti-phage activity” or “resistant to infection by at least one phage” or “resistance to at least one phage” or “anti-phage defense” encompasses an activity provided by a Defense system to a host cell, for example but not limited to bacterial cell expressing a functional Defense System disclosed herein, wherein said bacterial cell then comprises an increased resistance to infection by at least one phage in comparison to bacteria of the same species under the same developmental stage (e.g. culture state) which does not express an endogenous functional Defense System. Resistance to infection, may be determined by for example but not limited to bacterial viability, phage lysogeny, phage genomic replication, or phage genomic degradation. The phage can be a lytic phage or a temperate (lysogenic) phage described herein. In some embodiments, the increase in resistance is at least 1.5 fold, at least 2 fold, at least 3 fold, at least 5 fold, at least 10 fold, or at least 20 fold as compared to same bacterial host in the absence of the functional Defense System.

In some embodiments, the increase in resistance is by at least 5%, by at least a 10%, at least 20%, at least 30%, at least 40%, at least 50%, at least 60%, at least 70%, at least 80%, at least 90% or more than 100% as compared to same host bacteria in the absence of the functional Defense System.

Assays for testing phage resistance are well known in the art and are further described hereinbelow.

A skilled artisan would appreciate that the terms “anti-plasmid activity” or “defense against plasmid transformation” or “reduced transformation by a plasmid” or “anti-plasmid defense” or “plasmid resistance” encompasses an activity provided by a Defense System to a host cell, for example but not limited to bacterial cell expressing a functional Defense System disclosed herein, wherein the bacterial cell then comprises a decreased efficiency of transformation by at least one plasmid in comparison to bacteria of the same species under the same developmental stage (e.g. culture state) which does not express a functional Defense System. Decreased efficiency of plasmid transformation, may be determined by for example but not limited to a transformation efficiency assay comparing bacteria comprising a Defense System with those not comprising the Defense System. The plasmid may be an episomal plasmid. In some embodiments, the decreased transformation efficiency is at least 1.5 fold, at least 2 fold, at least 3 fold, at least 5 fold, at least 10 fold, or at least 20 fold as compared to same bacterial host in the absence of the functional Defense System.

In some embodiments, the decreased transformation efficiency is by at least 5%, by at least a 10%, at least 20%, at least 30%, at least 40%, at least 50%, at least 60%, at least 70%, at least 80%, at least 90% or more than 100% as compared to same host bacteria in the absence of the functional Defense System.

A skilled artisan would appreciate that the term “donor species” may in certain embodiments, encompass the bacterial or archaeal species in which a Defense System was identified and sequenced. The skilled artisan would also appreciate that the term “host cell” may in certain embodiments, encompass bacterial or archaeal or eukaryotic cell in which a Defense System has been introduced. In some embodiments, the host cell does not endogenously comprise the Defense System introduced. In some embodiments, the host cell does not endogenously comprise a functional version of the Defense System introduced. In some embodiments, the host cell comprises the Defense System introduced but it is not functional in the host cell. In some embodiments, the host cell does not endogenously express the Defense System introduced. In some embodiments, the host cell does not endogenously express a functional version of the Defense System introduced.

Regardless of the method of introduction, in some embodiments provided herein are cells, for example but not limited to bacterial cells, which comprise a heterologous functional defense system, or components thereof. In some embodiments, the method of introduction is conjugation.

In some embodiments, the isolated cell is transformed or transfected with the described nucleic acid construct or nucleic acid construct Defense System, or a combination thereof.

In some embodiments, in some embodiments a combination of Defense Systems comprises a combination of the nucleic acid constructs comprising each Defense System. In some embodiments a combination of Defense systems comprises a combination of the nucleic acid constructs comprising a functional Defense System selected from Defense System Ia, Ib, II, IIIa, IIIb, IIIc, IV, V, VI, VII, VIII, IX, Xa, Xb, and Xc. In some embodiments two defense systems are combined. In some embodiments 2-17 defense systems are combined. In some embodiments 2-5 defense systems are combined. In some embodiments 2-10 defense systems are combined. In some embodiments 5-10 defense systems are combined. In some embodiments 10-17 defense systems are combined. In some embodiments, 2, 3, 4, 5, 6, 7, 8, 9, 10, 11, 12, 13, 14, 15, 16, 17 defense systems are combined. In some embodiments 2-40 defense systems are combined. In some embodiments, multiple copies of the same Defense System are combined. In some embodiments, the combination comprises all different Defense Systems. In some embodiments a combination of Defense systems comprises any combination of the nucleic acid constructs comprising a functional Defense System selected from Defense System Ia, Ib, II, IIIa, IIIb, IIIc, IV, V, VI, VII, VIII, IX, Xa, Xb, and Xc.

In some embodiments, a combination comprising a Defense System Ia comprises a combination of Defense System Ia with any combination of Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 1-16 defense systems selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 1 defense system selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 2 defense systems selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 3 defense systems selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 4 defense systems selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 5 defense systems selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 6 defense systems selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 7 defense systems selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 8 defense systems selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 9 defense systems selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 10 defense systems selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 11 defense systems selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 12 defense systems selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 13 defense systems selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 14 defense systems selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 15 defense systems selected from Defense Systems Ib, II, IIIa, IIIb, IIIc, IV, V, VI, VII, VIII, IX, Xa, Xb, and Xc. In some embodiments, a combination comprising a Defense System Ia comprises a combination of Defense System Ia with 16 defense systems selected from Defense Systems Ib, II, IIIa, IIIb, IIIc, IV, V, VI, VII, VIII, IX, Xa, Xb, and Xc.

In some embodiments, a combination comprising a Defense System Ib comprises a combination of Defense System Ib with any combination of Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ia with 1-16 defense systems selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ia with 1 defense system selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ib with 2 defense systems selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ib with 3 defense systems selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ib with 4 defense systems selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ib with 5 defense systems selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ib with 6 defense systems selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ib with 7 defense systems selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ib with 8 defense systems selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ib with 9 defense systems selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ib with 10 defense systems selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ib with 11 defense systems selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ib with 12 defense systems selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ib with 13 defense systems selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ib with 14 defense systems selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ib with 15 defense systems selected from Defense Systems Ia, II, IIIa, IIIb, IIIc, IV, V, VI, VII, VIII, IX, Xa, Xb, and Xc. In some embodiments, a combination comprising a Defense System Ib comprises a combination of Defense System Ib with 16 defense systems selected from Defense Systems Ia, II, IIIa, IIIb, IIIc, IV, V, VI, VII, VIII, IX, Xa, Xb, and Xc.

In some embodiments, a combination comprising a Defense System II comprises a combination of Defense System II with any combination of Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 1-16 defense systems selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 1 defense system selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 2 defense systems selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 3 defense systems selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 4 defense systems selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 5 defense systems selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 6 defense systems selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 7 defense systems selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 8 defense systems selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 9 defense systems selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 10 defense systems selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 11 defense systems selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 12 defense systems selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 13 defense systems selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 14 defense systems selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 15 defense systems selected from Defense Systems Ia, Ib, IIIa, IIIb, IIIc, IV, V, VI, VII, VIII, IX, Xa, Xb, and Xc. In some embodiments, a combination comprising a Defense System II comprises a combination of Defense System II with 16 defense systems selected from Defense Systems Ia, Ib, IIIa, IIIb, IIIc, IV, V, VI, VII, VIII, IX, Xa, Xb, and Xc.

In some embodiments, a combination comprising a Defense System IIIa comprises a combination of Defense System IIIa with any combination of Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 1-16 defense systems selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 1 defense system selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 2 defense systems selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 3 defense systems selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 4 defense systems selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 5 defense systems selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 6 defense systems selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 7 defense systems selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 8 defense systems selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 9 defense systems selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 10 defense systems selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 11 defense systems selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 12 defense systems selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 13 defense systems selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 14 defense systems selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 15 defense systems selected from Defense Systems Ia, Ib, II, IIIb, IIIc, IV, V, VI, VII, VIII, IX, Xa, Xb, and Xc. In some embodiments, a combination comprising a Defense System IIIa comprises a combination of Defense System IIIa with 16 defense systems selected from Defense Systems Ia, Ib, II, IIIb, IIIc, IV, V, VI, VII, VIII, IX, Xa, Xb, and Xc.

In some embodiments, a combination comprising a Defense System IIIb comprises a combination of Defense System IIIb with any combination of Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 1-16 defense systems selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 1 defense system selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 2 defense systems selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 3 defense systems selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 4 defense systems selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 5 defense systems selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 6 defense systems selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 7 defense systems selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 8 defense systems selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 9 defense systems selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 10 defense systems selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 11 defense systems selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 12 defense systems selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 13 defense systems selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 14 defense systems selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 15 defense systems selected from Defense Systems Ia, Ib, II, IIIa, IIIc, IV, V, VI, VII, VIII, IX, Xa, Xb, and Xc. In some embodiments, a combination comprising a Defense System IIIb comprises a combination of Defense System IIIb with 16 defense systems selected from Defense Systems Ia, Ib, II, IIIa, IIIc, IV, V, VI, VII, VIII, IX, Xa, Xb, and Xc.

In some embodiments, a combination comprising a Defense System IIIc comprises a combination of Defense System IIIc with any combination of Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 1-16 defense systems selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 1 defense system selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 2 defense systems selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 3 defense systems selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 4 defense systems selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 5 defense systems selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 6 defense systems selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 7 defense systems selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 8 defense systems selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 9 defense systems selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 10 defense systems selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 11 defense systems selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 12 defense systems selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 13 defense systems selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 14 defense systems selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 15 defense systems selected from Defense Systems Ia, Ib, II, IIIa, IIIb, IV, V, VI, VII, VIII, IX, Xa, Xb, and Xc. In some embodiments, a combination comprising a Defense System IIIc comprises a combination of Defense System IIIc with 16 defense systems selected from Defense Systems Ia, Ib, II, IIIa, IIIb, IV, V, VI, VII, VIII, IX, Xa, Xb, and Xc.

In some embodiments, a combination comprising a Defense System IV comprises a combination of Defense System IV with any combination of Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 1-16 defense systems selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 1 defense system selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 2 defense systems selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 3 defense systems selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 4 defense systems selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 5 defense systems selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 6 defense systems selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 7 defense systems selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 8 defense systems selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 9 defense systems selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 10 defense systems selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 11 defense systems selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 12 defense systems selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 13 defense systems selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 14 defense systems selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 15 defense systems selected from Defense Systems Ia, Ib, II, IIIa, IIIb, IIIc, V, VI, VII, VIII, IX, Xa, Xb, and Xc. In some embodiments, a combination comprising a Defense System IV comprises a combination of Defense System IV with 16 defense systems selected from Defense Systems Ia, Ib, II, IIIa, IIIb, IIIc, V, VI, VII, VIII, IX, Xa, Xb, and Xc.

In some embodiments, a combination comprising a Defense System V comprises a combination of Defense System V with any combination of Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 1-16 defense systems selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 1 defense system selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 2 defense systems selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 3 defense systems selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 4 defense systems selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 5 defense systems selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 6 defense systems selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 7 defense systems selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 8 defense systems selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 9 defense systems selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 10 defense systems selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 11 defense systems selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 12 defense systems selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 13 defense systems selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 14 defense systems selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 15 defense systems selected from Defense Systems Ia, Ib, II, IIIa, IIIb, IIIc, IV, VI, VII, VIII, IX, Xa, Xb, and Xc. In some embodiments, a combination comprising a Defense System V comprises a combination of Defense System V with 16 defense systems selected from Defense Systems Ia, Ib, II, IIIa, IIIb, IIIc, IV, VI, VII, VIII, IX, Xa, Xb, and Xc.

In some embodiments, a combination comprising a Defense System VI comprises a combination of Defense System VI with any combination of Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 1-16 defense systems selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 1 defense system selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 2 defense systems selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 3 defense systems selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 4 defense systems selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 5 defense systems selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 6 defense systems selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 7 defense systems selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 8 defense systems selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 9 defense systems selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 10 defense systems selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 11 defense systems selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 12 defense systems selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 13 defense systems selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 14 defense systems selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 15 defense systems selected from Defense Systems Ia, Ib, II, IIIa, IIIb, IIIc, IV, V, VII, VIII, IX, Xa, Xb, and Xc. In some embodiments, a combination comprising a Defense System VI comprises a combination of Defense System VI with 16 defense systems selected from Defense Systems Ia, Ib, II, IIIa, IIIb, IIIc, IV, V, VII, VIII, IX, Xa, Xb, and Xc.

In some embodiments, a combination comprising a Defense System VII comprises a combination of Defense System VII with any combination of Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 1-16 defense systems selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 1 defense system selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 2 defense systems selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 3 defense systems selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 4 defense systems selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 5 defense systems selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 6 defense systems selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 7 defense systems selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 8 defense systems selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 9 defense systems selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 10 defense systems selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 11 defense systems selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 12 defense systems selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 13 defense systems selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 14 defense systems selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 15 defense systems selected from Defense Systems Ia, Ib, II, IIIa, IIIb, IIIc, IV, VI, V, VI, VIII, IX, Xa, Xb, and Xc. In some embodiments, a combination comprising a Defense System VII comprises a combination of Defense System VII with 16 defense systems selected from Defense Systems Ia, Ib, II, IIIa, IIIb, IIIc, IV, VI, V, VI, VIII, IX, Xa, Xb, and Xc.

In some embodiments, a combination comprising a Defense System VIII comprises a combination of Defense System VIII with any combination of Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 1-16 defense systems selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 1 defense system selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 2 defense systems selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 3 defense systems selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 4 defense systems selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 5 defense systems selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 6 defense systems selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 7 defense systems selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 8 defense systems selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 9 defense systems selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 10 defense systems selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 11 defense systems selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 12 defense systems selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 13 defense systems selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 14 defense systems selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 15 defense systems selected from Defense Systems Ia, Ib, II, IIIa, IIIb, IIIc, IV, VI, V, VI, VII, IX, Xa, Xb, and Xc. In some embodiments, a combination comprising a Defense System VIII comprises a combination of Defense System VIII with 16 defense systems selected from Defense Systems Ia, Ib, II, IIIa, IIIb, IIIc, IV, VI, V, VI, VII, IX, Xa, Xb, and Xc.

In some embodiments, a combination comprising a Defense System IX comprises a combination of Defense System IX with any combination of Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 1-16 defense systems selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 1 defense system selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 2 defense systems selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 3 defense systems selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 4 defense systems selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 5 defense systems selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 6 defense systems selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 7 defense systems selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 8 defense systems selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 9 defense systems selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 10 defense systems selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 11 defense systems selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 12 defense systems selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 13 defense systems selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 14 defense systems selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 15 defense systems selected from Defense Systems Ia, Ib, II, IIIa, IIIb, IIIc, IV, VI, V, VI, VII, VIII, Xa, Xb, and Xc. In some embodiments, a combination comprising a Defense System IX comprises a combination of Defense System IX with 16 defense systems selected from Defense Systems Ia, Ib, II, IIIa, IIIb, IIIc, IV, VI, V, VI, VII, VIII, Xa, Xb, and Xc.

In some embodiments, a combination comprising a Defense System Xb comprises a combination of Defense System Xb with any combination of Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 1-16 defense systems selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 1 defense system selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 2 defense systems selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 3 defense systems selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 4 defense systems selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 5 defense systems selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 6 defense systems selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 7 defense systems selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 8 defense systems selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 9 defense systems selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 10 defense systems selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 11 defense systems selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 12 defense systems selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 13 defense systems selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 14 defense systems selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 15 defense systems selected from Defense Systems Ia, Ib, II, IIIa, IIIb, IIIc, IV, VI, V, VI, VII, VIII, IX, Xa, and Xc. In some embodiments, a combination comprising a Defense System Xb comprises a combination of Defense System Xb with 16 defense systems selected from Defense Systems Ia, Ib, II, IIIa, IIIb, IIIc, IV, VI, V, VI, VII, VIII, IX, Xa, and Xc.

In some embodiments, a combination comprising a Defense System Xc comprises a combination of Defense System Xc with any combination of Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 1-16 defense systems selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 1 defense system selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 2 defense systems selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 3 defense systems selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 4 defense systems selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 5 defense systems selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 6 defense systems selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 7 defense systems selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 8 defense systems selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 9 defense systems selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 10 defense systems selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 11 defense systems selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 12 defense systems selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 13 defense systems selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 14 defense systems selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 15 defense systems selected from Defense Systems Ia, Ib, II, IIIa, IIIb, IIIc, IV, VI, V, VI, VII, VIII, IX, Xa, and Xb. In some embodiments, a combination comprising a Defense System Xc comprises a combination of Defense System Xc with 16 defense systems selected from Defense Systems Ia, Ib, II, IIIa, IIIb, IIIc, IV, VI, V, VI, VII, VIII, IX, Xa, and Xb.

In some embodiments, a combination of Defense Systems disclosed herein above, does not naturally occur in nature. In some embodiments, a combination of Defense Systems disclosed herein above, does not naturally occur in a host cell. In some embodiments, a combination of Defense Systems disclosed herein above, does not naturally occur in bacterial cell. In some embodiments, a combination of Defense Systems disclosed herein above, does not naturally occur in an archaeal cell. In some embodiments, a combination of Defense Systems disclosed herein above, does not naturally occur in a eukaryotic cell. In some embodiments, a combination of Defense Systems disclosed herein above, does not naturally occur in a species disclosed in Tables 6-19C. In some embodiments, a combination of Defense Systems disclosed herein above, does not naturally occur in a host cell.

In some embodiments, a combination of Defense Systems disclosed herein above, is not expressed together in nature. In some embodiments, a combination of Defense Systems disclosed herein above, is not naturally expressed in a host cell. In some embodiments, a combination of Defense Systems disclosed herein above, is not naturally expressed in bacterial cell. In some embodiments, a combination of Defense Systems disclosed herein above, is not naturally expressed in an archaeal cell. In some embodiments, a combination of Defense Systems disclosed herein above, is not naturally expressed in a eukaryotic cell. In some embodiments, a combination of Defense Systems disclosed herein above, is not naturally expressed in a species disclosed in Tables 6-17.

In some embodiments, a combination of functional Defense Systems disclosed herein above, does not naturally occur in nature. In some embodiments, a combination of functional Defense Systems disclosed herein above, does not naturally occur in a host cell. In some embodiments, a combination of functional Defense Systems disclosed herein above, does not naturally occur in bacterial cell. In some embodiments, a combination of functional Defense Systems disclosed herein above, does not naturally occur in an archaeal cell. In some embodiments, a combination of functional Defense Systems disclosed herein above, does not naturally occur in a eukaryotic cell. In some embodiments, a combination of functional Defense Systems disclosed herein above, does not naturally occur in a species disclosed in Tables 6-19C. In some embodiments, a combination of functional Defense Systems disclosed herein above, does not naturally occur in a host cell.

In some embodiments, a cell comprising a combination of Defense Systems disclosed herein above, does not naturally occur in nature. In some embodiments, a cell comprising a combination of functional Defense Systems disclosed herein above, does not naturally occur in nature.

In some embodiments, a nucleic acid constructs, comprised in a Defense System Ia, Ib, II, IIIa, IIIb, IIIc, IV, V, VI, VII, VIII, IX, Xa, Xb, or Xc, encodes a polycistronic mRNA comprising said nucleic acid sequences. In some embodiments, a nucleic acid constructs, comprised in a Defense System Ia, Ib, II, IIIa, IIIb, IIIc, IV, V, VI, VII, VIII, IX, Xa, Xb, or Xc, encodes a polycistronic mRNA comprising said nucleic acid sequences encoding said polypeptides. In some embodiments, a nucleic acid constructs, comprised in a Defense System Ia, Ib, II, IIIa, IIIb, IIIc, IV, V, VI, VII, VIII, IX, Xa, Xb, or Xc, encodes a polycistronic mRNA comprising said nucleic acid sequences encoding components or functional portions thereof, of a Defense System.

Various construct schemes can be utilized to express few genes from a single nucleic acid construct. In some embodiments, the construct encodes a polycistronic mRNA comprising the nucleic acid sequences. In some embodiments, the nucleic acid sequences can be co-transcribed as a polycistronic message from a single promoter sequence of the nucleic acid construct. To enable co-translation of all the genes from a single polycistronic message, the different nucleic acid sequence segments can be transcriptionally fused via linker sequence including an internal ribosome entry site (IRES) sequence, which enables the translation of the nucleic acid sequence segment downstream of the IRES sequence. In this case, a transcribed polycistronic RNA molecule including the coding sequences of different combinations of the nucleic acid sequences, for example as disclosed herein, will be translated from both the capped 5′ end and the internal IRES sequence of the polycistronic RNA molecule.

In some embodiments, each two nucleic acid sequence segments can be translationally fused via protease recognition site cleavable by a protease expressed by the cell to be transformed with the nucleic acid construct. In this case, a chimeric polypeptide translated will be cleaved by the cell expressed protease.

In some embodiments, the nucleic acid construct can include at least two Cis acting regulatory elements each being for separately expressing a distinct nucleic acid sequence. These at least two Cis acting regulatory elements can be identical or distinct.

In some embodiments, disclosed herein is an isolated polypeptide comprising an amino acid sequence comprising one component of a Defense System disclosed herein. In some embodiments, disclosed herein is an isolated polypeptide comprising an amino acid sequence comprising two components of a Defense System disclosed herein. In some embodiments, disclosed herein is an isolated polypeptide comprising an amino acid sequence comprising three components of a Defense System disclosed herein. In some embodiments, disclosed herein is an isolated polypeptide comprising an amino acid sequence comprising four components of a Defense System disclosed herein. In some embodiments, disclosed herein is an isolated polypeptide comprising an amino acid sequence comprising five components of a Defense System disclosed herein.

In some embodiments, disclosed herein is an isolated polypeptide comprising an amino acid sequence comprising one component of a Defense System Ia or a Defense System Ib, said component selected from the group consisting of a ZorA polypeptide, a ZorB polypeptide, a ZorC polypeptide, a ZorD polypeptide, and a ZorE polypeptide. In some embodiments, is an isolated polypeptide comprising an amino acid sequence comprising two components of a Defense System Ia or a Defense System Ib, said component selected from the group consisting of a ZorA polypeptide, a ZorB polypeptide, a ZorC polypeptide, a ZorD polypeptide, and a ZorE polypeptide. In some embodiments, is an isolated polypeptide comprising an amino acid sequence comprising three components of a Defense System Ia or a Defense System Ib, said component selected from the group consisting of a ZorA polypeptide, a ZorB polypeptide, a ZorC polypeptide, a ZorD polypeptide, and a ZorE polypeptide. In some embodiments, is an isolated polypeptide comprising an amino acid sequence comprising four components of a Defense System Ia or a Defense System Ib, said component selected from the group consisting of a ZorA polypeptide, a ZorB polypeptide, a ZorC polypeptide, a ZorD polypeptide, and a ZorE polypeptide. In some embodiments, is an isolated polypeptide comprising an amino acid sequence comprising greater than four components of a Defense System Ia or a Defense System Ib, said component selected from the group consisting of a ZorA polypeptide, a ZorB polypeptide, a ZorC polypeptide, a ZorD polypeptide, and a ZorE polypeptide.

In some embodiments, disclosed herein is an isolated polypeptide comprising an amino acid sequence comprising one component of a Defense System II, said component selected from the group consisting of a ThsA polypeptide and a ThsB polypeptide. In some embodiments, is an isolated polypeptide comprising an amino acid sequence comprising two components of a Defense System II, said component selected from the group consisting of a ThsA polypeptide and a ThsB polypeptide. In some embodiments, is an isolated polypeptide comprising an amino acid sequence comprising more than two components of a Defense System II, said component selected from the group consisting of a ThsA polypeptide and a ThsB polypeptide.

In some embodiments, disclosed herein is an isolated polypeptide comprising an amino acid sequence comprising one component of a Defense System IIIa, said component selected from the group consisting of a DruA polypeptide, a DruB polypeptide, a DruC polypeptide, a DruD polypeptide, a DruE polypeptide. In some embodiments, is an isolated polypeptide comprising an amino acid sequence comprising two components of a Defense System IIIa, said component selected from the group consisting of a DruA polypeptide, a DruB polypeptide, a DruC polypeptide, a DruD polypeptide, a DruE polypeptide. In some embodiments, is an isolated polypeptide comprising an amino acid sequence comprising three components of a Defense System IIIa, said component selected from the group consisting of a DruA polypeptide, a DruB polypeptide, a DruC polypeptide, a DruD polypeptide, a DruE polypeptide. In some embodiments, is an isolated polypeptide comprising an amino acid sequence comprising four components of a Defense System IIIa, said component selected from the group consisting of a DruA polypeptide, a DruB polypeptide, a DruC polypeptide, a DruD polypeptide, a DruE polypeptide. In some embodiments, is an isolated polypeptide comprising an amino acid sequence comprising five components of a Defense System IIIa, said component selected from the group consisting of a DruA polypeptide, a DruB polypeptide, a DruC polypeptide, a DruD polypeptide, a DruE polypeptide. In some embodiments, is an isolated polypeptide comprising an amino acid sequence comprising more than five components of a Defense System IIIa, said component selected from the group consisting of a DruA polypeptide, a DruB polypeptide, a DruC polypeptide, a DruD polypeptide.

In some embodiments, disclosed herein is an isolated polypeptide comprising an amino acid sequence comprising one component of a Defense System IIIb, said component selected from the group consisting of a DruM polypeptide, a DruF polypeptide, a DruG polypeptide, a DruE polypeptide. In some embodiments, is an isolated polypeptide comprising an amino acid sequence comprising two components of a Defense System IIIb, said component selected from the group consisting of a DruM polypeptide, a DruF polypeptide, a DruG polypeptide, a DruE polypeptide. In some embodiments, is an isolated polypeptide comprising an amino acid sequence comprising three components of a Defense System IIIb, said component selected from the group consisting of a DruM polypeptide, a DruF polypeptide, a DruG polypeptide, a DruE polypeptide. In some embodiments, is an isolated polypeptide comprising an amino acid sequence comprising four components of a Defense System IIIb, said component selected from the group consisting of a DruM polypeptide, a DruF polypeptide, a DruG polypeptide, a DruE polypeptide. In some embodiments, is an isolated polypeptide comprising an amino acid sequence comprising five components of a Defense System IIIb, said component selected from the group consisting of a DruM polypeptide, a DruF polypeptide, a DruG polypeptide, a DruE polypeptide. In some embodiments, is an isolated polypeptide comprising an amino acid sequence comprising more than five components of a Defense System IIIb, said component selected from the group consisting of a DruM polypeptide, a DruF polypeptide, a DruG polypeptide, a DruE polypeptide.

In some embodiments, disclosed herein is an isolated polypeptide comprising an amino acid sequence comprising one component of a Defense System IIIc, said component selected from the group consisting of a DruH polypeptide a DruE polypeptide. In some embodiments, is an isolated polypeptide comprising an amino acid sequence comprising two components of a Defense System IIIc, said component selected from the group consisting of a DruH polypeptide a DruE polypeptide. In some embodiments, is an isolated polypeptide comprising an amino acid sequence comprising three components of a Defense System IIIc, said component selected from the group consisting of a DruH polypeptide a DruE polypeptide. In some embodiments, is an isolated polypeptide comprising an amino acid sequence comprising four components of a Defense System IIIc, said component selected from the group consisting of a DruH polypeptide a DruE polypeptide. In some embodiments, is an isolated polypeptide comprising an amino acid sequence comprising five components of a Defense System IIIc, said component selected from the group consisting of a DruH polypeptide a DruE polypeptide. In some embodiments, is an isolated polypeptide comprising an amino acid sequence comprising more than five components of a Defense System IIIc, said component selected from the group consisting of a DruH polypeptide a DruE polypeptide.

In some embodiments, disclosed herein is an isolated polypeptide comprising an amino acid sequence comprising one component of a Defense System IV, said component selected from the group consisting of a HamA polypeptide and a HamB polypeptide. In some embodiments, is an isolated polypeptide comprising an amino acid sequence comprising two components of a Defense System IV, said component selected from the group consisting of a HamA polypeptide and a HamB polypeptide. In some embodiments, is an isolated polypeptide comprising an amino acid sequence comprising more than two components of a Defense System IV, said component selected from the group consisting of a HamA polypeptide and a HamB polypeptide.

In some embodiments, disclosed herein is an isolated polypeptide comprising an amino acid sequence comprising one component of a Defense System V, said component selected from the group consisting of a SduA polypeptide. In some embodiments, is an isolated polypeptide comprising an amino acid sequence comprising more than one component of a Defense System V, said component selected from the group consisting of a SduA polypeptide.

In some embodiments, disclosed herein is an isolated polypeptide comprising an amino acid sequence comprising one component of a Defense System VI, said component selected from the group consisting of a GajA polypeptide and a GajB polypeptide. In some embodiments, is an isolated polypeptide comprising an amino acid sequence comprising two components of a Defense System VI, said component selected from the group consisting of a GajA polypeptide and a GajB polypeptide. In some embodiments, is an isolated polypeptide comprising an amino acid sequence comprising more than two components of a Defense System VI, said component selected from the group consisting of a GajA polypeptide and a GajB polypeptide.

In some embodiments, disclosed herein is an isolated polypeptide comprising an amino acid sequence comprising one component of a Defense System VII, said component selected from the group consisting of a PtuA polypeptide and a PtuB polypeptide. In some embodiments, is an isolated polypeptide comprising an amino acid sequence comprising two components of a Defense System VII, said component selected from the group consisting of a PtuA polypeptide and a PtuB polypeptide. In some embodiments, is an isolated polypeptide comprising an amino acid sequence comprising more than two components of a Defense System VII, said component selected from the group consisting of a PtuA polypeptide and a PtuB polypeptide.

In some embodiments, disclosed herein is an isolated polypeptide comprising an amino acid sequence comprising one component of a Defense System VIII, said component selected from the group consisting of a LmuA polypeptide and a LmuB polypeptide. In some embodiments, is an isolated polypeptide comprising an amino acid sequence comprising two components of a Defense System VIII, said component selected from the group consisting of a LmuA polypeptide and a LmuB polypeptide. In some embodiments, is an isolated polypeptide comprising an amino acid sequence comprising more than two components of a Defense System VIII, said component selected from the group consisting of a LmuA polypeptide and a LmuB polypeptide.

In some embodiments, disclosed herein is an isolated polypeptide comprising an amino acid sequence comprising one component of a Defense System IX, said component selected from the group consisting of a KwaA polypeptide and a KwaB polypeptide. In some embodiments, is an isolated polypeptide comprising an amino acid sequence comprising two components of a Defense System IX, said component selected from the group consisting of a KwaA polypeptide and a KwaB polypeptide. In some embodiments, is an isolated polypeptide comprising an amino acid sequence comprising more than two components of a Defense System IX, said component selected from the group consisting of a KwaA polypeptide and a KwaB polypeptide.

In some embodiments, disclosed herein is an isolated polypeptide comprising an amino acid sequence comprising one component of a Defense System Xa or a Defense System Xb or a Defense System Xc, said component selected from the group consisting of a JetA polypeptide, a JetB polypeptide, a JetC polypeptide, and a JetD polypeptide, and variants thereof. In some embodiments, is an isolated polypeptide comprising an amino acid sequence comprising two components of a Defense System Xa or a Defense System Xb or a Defense System Xc, said component selected from the group consisting of a JetA polypeptide, a JetB polypeptide, a JetC polypeptide, and a JetD polypeptide, and variants thereof. In some embodiments, is an isolated polypeptide comprising an amino acid sequence comprising three components of a Defense System Xa or a Defense System Xb or a Defense System Xc, said component selected from the group consisting of a JetA polypeptide, a JetB polypeptide, a JetC polypeptide, and a JetD polypeptide, and variants thereof. In some embodiments, is an isolated polypeptide comprising an amino acid sequence comprising four components of a Defense System Xa or a Defense System Xb or a Defense System Xc, said component selected from the group consisting of a JetA polypeptide, a JetB polypeptide, a JetC polypeptide, and a JetD polypeptide, and variants thereof. In some embodiments, is an isolated polypeptide comprising an amino acid sequence comprising more than four components of a Defense System Xa or a Defense System Xb or a Defense System Xc, said component selected from the group consisting of a JetA polypeptide, a JetB polypeptide, a JetC polypeptide, and a JetD polypeptide, and variants thereof.

In some embodiments, the isolated polypeptide comprising an amino acid sequence of a Defense System has an anti-phage activity. In some embodiments, the isolated polypeptide comprising an amino acid sequence of a Defense System has an anti-plasmid activity. In some embodiments, the isolated polypeptide comprising an amino acid sequence of a Defense System has an anti-phage activity and an anti-plasmid activity. In some embodiments, the anti-phage activity is against at least one phage. In some embodiments, the anti-phage activity is against more than one phage.

In some embodiments, any one of said constructs disclosed herein, comprises multiple nucleic acid sequences, each sequence encoding at least one of said polypeptides. In some embodiments, any one of said constructs disclosed herein, comprises multiple nucleic acid sequences, each sequence encoding at least a functional portion of one of said polypeptides. In some embodiments, any one of said constructs disclosed herein, comprises multiple nucleic acid sequences, each sequence encoding at least one component of a Defense System. In some embodiments, any one of said constructs disclosed herein, comprises multiple nucleic acid sequences, each sequence encoding at least one functional component of a Defense System. In some embodiments, any one of said constructs disclosed herein, comprises multiple nucleic acid sequences, each sequence encoding at least one functional portion of a component of a Defense System.

As used herein, the terms designating a polypeptide, for example the terms “ZorA”, “ZorB”, “ZorC”, “ZorD”, “ZorE”, “ThsA”, “ThsB”, “DruA”, “DruB”, “DruC”, “DruD”, “DruE”, DruM”, “DruF”, “DruG”, “DruH”, “HamA”, “HamB”, “SduA”, “GajA”, “GajB”, “PtuA”, “PtuB”, “LmuA”, “LmuB”, “KwaA”, “KwaB”, “JetA”, “JetB”, “JetC”, “JetD”, “JetAⁱⁱ”, “JetBⁱⁱ”, “JetCⁱⁱ”, “JetDⁱⁱ”, “JetAiⁱⁱ”, “JetBⁱⁱⁱ”, “JetCⁱⁱⁱ”, and “JetDⁱⁱⁱ”, in some embodiments comprise full length polypeptides, respectively. In some embodiments, the terms designating a polypeptide, for example the terms “ZorA”, “ZorB”, “ZorC”, “ZorD”, “ZorE”, “ThsA”, “ThsB”, “DruA”, “DruB”, “DruC”, “DruD”, “DruE”, DruM”, “DruF”, “DruG”, “DruH”, “HamA”, “HamB”, “SduA”, “GajA”, “GajB”, “PtuA”, “PtuB”, “LmuA”, “LmuB”, “KwaA”, “KwaB”, “JetA”, “JetB”, “JetC”, “JetD”, “JetAⁱⁱ”, “JetBⁱⁱ” “JetCⁱⁱ” “JetDⁱⁱ”, “JetAiⁱⁱ”, “JetBⁱⁱⁱ”, “JetCⁱⁱⁱ”, and “JetDⁱⁱⁱ”, comprise a fragment of the full length polypeptide, wherein the fragment maintains the activity as disclosed herein.

Similarly, the terms designating a gene, for example the terms “zorA”, “zorB”, “zorC”, “zorD”, “zorE”, “thsA”, “thsB”, “druA”, “druB”, “druC”, “druD”, “druE”, “druM”, “druF”, “druG”, “druH”, “hamA”, “hamB”, “sduA”, “gajA”, “gajB”, “ptuA”, “ptuB”, “lmuA”, “lmuB”, “kwaA”, “kwaB”, “jetA”, “jetB”, “jetC”, “jetD”, “jetAⁱⁱ”, “jetBⁱⁱ”, “jetCⁱⁱ”, “jetDⁱ”, “jetAⁱⁱⁱ”, “jetBⁱⁱⁱ”, “jetCⁱⁱⁱ”, and “jetDⁱⁱⁱ”, in some embodiments comprise a full length gene, respectively. In some embodiments, the terms designating a gene, for example the terms “zorA”, “zorB”, “zorC”, “zorD”, “zorE”, “thsA”, “thsB”, “druA”, “druB”, “druC”, “druD”, “druE”, “druM”, “druF”, “druG”, “druH”, “hamA”, “hamB”, “sduA”, “gajA”, “gajB”, “ptuA”, “ptuB”, “lmuA”, “lmuB”, “kwaA”, “kwaB”, “jetA”, “jetB”, “jetC”, “jetD”, “jetAⁱⁱ”, “jetBⁱⁱ”, “jetCⁱⁱ”, “jetDⁱⁱ”, “jetAⁱⁱⁱ”, “jetBⁱⁱⁱ”, “jetCⁱⁱⁱ”, and “jetDⁱⁱⁱ”, comprise a fragment of the full length gene, wherein the fragment encodes a polypeptide, respectively, comprising the activity as disclosed herein.

In some embodiments, a construct comprising the nucleic acid sequence encoding the ZorA polypeptide, the ZorB polypeptide, the ZorC polypeptide, and the ZorD polypeptide, comprises a nucleic acid sequence having at least 80% homology with the sequence referenced in Table 8 rows 2-1174 columns G, AD, and AE or the sequence set forth in SEQ ID NO: 14, or comprises nucleic acid sequences having at least 80% homology to at least one of each of the sequences referenced in Table 8 rows 2-1830 columns J and L (encoding ZorA polypeptide), in Table 8 rows 2-1830 columns N and P (encoding ZorB polypeptide), in Table 8 rows 2-1174 columns R and T (encoding ZorC polypeptide), and in Table 8 rows 2-1174 columns V and X (encoding ZorD polypeptide).

In some embodiments, a construct comprising the nucleic acid sequence encoding the ZorA polypeptide, the ZorB polypeptide, and the ZorE polypeptide comprises a nucleic acid sequence having at least 80% homology to the sequence referenced in Table 8 rows 1175-1830 columns G, AD and AE, or the sequence set forth in SEQ ID NO: 15, or comprises nucleic acid sequences having at least 80% homology to the sequences referenced in Table 8 rows 2-1830 columns J and L (encoding ZorA polypeptide), in Table 8 rows 2-1830 columns N and P (encoding ZorB polypeptide), and in Table 8 rows 1175-1830 columns Z and AB (encoding ZorE polypeptide).

In some embodiments, a construct comprising the nucleic acid sequence encoding the ThsA polypeptide and the ThsB polypeptide comprises a nucleic acid sequence having at least 80% homology with the sequence referenced in Table 9 rows 2-2100 columns G, AG and AH, or the sequence set forth in SEQ ID NO: 10 or SEQ ID NO: 11, or comprises nucleic acid sequences having at least 80% homology to at least one of each of the sequences referenced in Table 9 rows 2-2100 columns I and K (encoding ThsA polypeptide) and the sequences referenced in Table 9 rows 2-2100 columns M and O, Q and S, U and W, Y and AA, and AC and AE (encoding ThsB polypeptide).

In some embodiments, a construct comprising the nucleic acid sequence encoding the DruA polypeptide, the DruB polypeptide, the DruC polypeptide, the DruD polypeptide, and the DruE polypeptide comprises a nucleic acid sequence having at least 80% homology with the sequences referenced in Table 10 rows 2-123 columns G, AT, and AU or the sequence set forth in SEQ ID NO: 16, or comprises nucleic acid sequences having at least 80% homology to at least one of each of the sequences referenced in Table 10 rows 2-123 columns J and L (encoding DruA polypeptide), referenced in Table 10 rows 2-123 columns N and P (encoding DruB polypeptide), referenced in Table 10 rows 2-123 columns R and T (encoding DruC polypeptide), referenced in Table 10 rows 2-123 columns V and X (encoding DruD polypeptide), and referenced in Table 10 rows 2-1343 columns Z and AB (encoding DruE polypeptide).

In some embodiments, a construct comprising the nucleic acid sequence encoding the DruM polypeptide, the DruF polypeptide, the DruG polypeptide, and the DruE polypeptide, comprises a nucleic acid sequence having at least 80% homology with the sequence referenced in Table 10 rows 124-295 columns G, AT, and AU, or comprises nucleic acid sequences having at least 80% homology to at least one of each of the sequences referenced in Table 10 rows 124-295 columns AD and AF (encoding DruM polypeptide), referenced in Table 10 rows 124-295 columns AH and AJ (encoding DruF polypeptide), referenced in Table 10 rows 124-295 columns AL and AN (encoding DruG polypeptide), and referenced in Table 10 rows 2-1343 columns Z and AB (encoding DruE polypeptide).

In some embodiments, a construct comprising the nucleic acid sequence encoding the DruH polypeptide and the DruE polypeptide, comprises a nucleic acid sequence having at least 80% homology with the sequence referenced in Table 10 rows 296-1343 columns G, AT, and AU, or comprises nucleic acid sequences having at least 80% homology to at least one of each of the sequences referenced in Table 10 rows 296-1343 columns AP and AR (encoding DruH polypeptide) and referenced in Table 10 rows 2-1343 columns Z and AB (encoding DruE polypeptide).

In some embodiments, a construct comprising the nucleic acid sequence encoding the HamA polypeptide and the HamB polypeptide comprises a nucleic acid sequence having at least 80% homology with the sequences referenced in Table 12 rows 2-1782 columns G, P, and Q or the sequence set forth in SEQ ID NO: 4, or comprises nucleic acid sequences having at least 80% homology to at least one of each of the sequences referenced in Table 12 rows 2-1782 column H and J (encoding HamA polypeptide) and referenced in Table 12 rows 2-1782 column L and N (encoding HamB polypeptide).

In some embodiments, a construct comprising the nucleic acid sequence encoding the SduA polypeptide comprises a nucleic acid sequence having at least 80% homology with the sequences referenced in Table 13 rows 2-1247 columns H and J or columns G, L, and M, or the sequences set forth in SEQ ID NO: 9.

In some embodiments, a construct comprising the nucleic acid sequence encoding the GajA polypeptide and the GajB polypeptide comprises a nucleic acid sequence having at least 80% homology with the sequences referenced in Table 14 rows 2-4599 columns G, P, and Q, or the sequence set forth in SEQ ID NO: 5 or SEQ ID NO: 6, or comprises nucleic acid sequences having at least 80% homology to at least one of each of sequences referenced in Table 14 rows 2-45-99 columns H and K (encoding GajA polypeptide) and referenced in Table 14 rows 2-45-99 columns L and N (encoding GajB polypeptide).

In some embodiments, a construct comprising the nucleic acid sequence encoding the PtuA polypeptide and the PtuB polypeptide comprises a nucleic acid sequence having at least 80% homology with the sequences referenced in Table 15 rows 2-2507 columns G, P and Q or the sequence set forth in SEQ ID NO: 12 or SEQ ID NO: 13, or comprises nucleic acid sequences having at least 80% homology to at least one of each of the sequences referenced in Table 15 rows 2-2507 columns H and J (encoding PtuA polypeptide) and referenced in Table 15 rows 2-2507 columns L and N (encoding PtuB polypeptide).

In some embodiments, a construct comprising the nucleic acid sequence encoding the LmuA polypeptide and the LmuB polypeptide comprises a nucleic acid sequence having at least 80% homology with the sequences referenced in Table 16 rows 2-698 columns G, P, and Q, or the sequence set forth in SEQ ID NO: 7 or SEQ ID NO: 8, or comprises nucleic acid sequences having at least 80% homology to at least one of each of the sequences referenced in Table 16 rows 2-698 column H (encoding LmuA polypeptide) and sequences referenced in Table 16 rows 2-698 column J (encoding LmuB polypeptide).

In some embodiments, a construct comprising the nucleic acid sequence encoding the KwaA polypeptide and the KwaB polypeptide comprises a nucleic acid sequence having at least 80% homology with the sequences referenced in Table 17 rows 2-935 columns G, P, and Q, or the sequence set forth in SEQ ID NO: 3, or comprises nucleic acid sequences having at least 80% homology to at least one of each of the sequences referenced in Table 17 rows 2-935 columns H and J (encoding KwaA polypeptide) and referenced in Table 17 rows 2-935 columns L and N (encoding KwaB polypeptide).

In some embodiments, a construct comprising the nucleic acid sequence encoding the JetA polypeptide, the JetB polypeptide, the JetC polypeptide, and the JetD comprises a nucleic acid sequence having at least 80% homology with the sequences referenced in Table 11 rows 2-2322 columns G, Z, and AA, or the sequence set forth in SEQ ID NO: 17, or comprises nucleic acid sequences having at least 80% homology to at least one of each of the sequences referenced in Table 11 rows 2-2322 columns J and L (encoding JetA polypeptide), referenced in Table 11 rows 2-2322 columns N and P (encoding JetB polypeptide), referenced in Table 11 rows 2-2322 columns R and T (encoding JetC polypeptide) and referenced in Table 11 rows 2-2322 columns V and X (encoding JetD polypeptide);

In some embodiments, a construct comprising the nucleic acid sequence encoding the JetAⁱⁱ polypeptide, the JetBⁱⁱ polypeptide, the JetCⁱⁱ polypeptide, and the JetDⁱⁱ comprises a nucleic acid sequence having at least 80% homology with the sequences referenced in Table 11 rows 2323-2844 columns G, Z, and AA, or the sequence set forth in SEQ ID NO: 18 or comprises nucleic acid sequences having at least 80% homology to at least one of each of the sequences referenced in Table 11 rows 2323-2844 columns J and L (encoding JetAⁱⁱ polypeptide), referenced in Table 11 rows 2323-2844 columns N and P (encoding JetBⁱⁱ polypeptide), referenced in Table 11 rows 2323-2844 columns R and T (encoding JetCⁱⁱ polypeptide) and referenced in Table 11 rows 2323-2844 columns V and X (encoding JetDⁱⁱ polypeptide);

In some embodiments, a construct comprising the nucleic acid sequence encoding the JetAⁱⁱⁱ polypeptide, the JetBⁱⁱⁱ polypeptide, the JetCⁱⁱⁱ polypeptide, and the JetDⁱⁱⁱ comprises a nucleic acid sequence having at least 80% homology with the sequences set forth in Table 11 rows 2845-3174 columns G, Z, and AA, or the sequence set forth in SEQ ID NO: 19 or comprises nucleic acid sequences having at least 80% homology to at least one of each of the sequences referenced in Table 11 rows 2845-3174 columns J and L (encoding JetAⁱⁱⁱ polypeptide), referenced in Table 11 rows 2845-3174 columns N and P (encoding JetBⁱⁱⁱ polypeptide), referenced in Table 11 rows 2845-3174 columns R and T (encoding JetC ill polypeptide) and referenced in Table 11 rows 2845-3174 columns V and X (encoding JetDⁱⁱⁱ polypeptide).

In some embodiments, a construct comprising a nucleic acid sequence comprises multiple copies of a nucleic acid sequence encoding a component of the Defense System. In some embodiments, a construct comprising a nucleic acid sequence comprises multiple copies of a nucleic acid sequence encoding two components of the Defense System. In some embodiments, a construct comprising a nucleic acid sequence comprises multiple copies of a nucleic acid sequence encoding three components of the Defense System. In some embodiments, a construct comprising a nucleic acid sequence comprises multiple copies of a nucleic acid sequence encoding four components of the Defense System. In some embodiments, a construct comprising a nucleic acid sequence comprises multiple copies of a nucleic acid sequence encoding five components of the Defense System. In some embodiments, a construct comprising a nucleic acid sequence comprises multiple copies of a nucleic acid sequence encoding more than components of the Defense System. In some embodiments, multiple copies comprise more than one copy. In some embodiments, multiple copies comprise 2 copies. In some embodiments, multiple copies comprise 2-10 copies. In some embodiments, multiple copies comprise 3 copies, 4 copies, 5 copies, 6 copies, 7 copies, 8 copies, 9 copies, or 10 copies.

In some embodiments, a Defense System disclosed herein provides a host cell with resistance to a foreign nucleic acid invasion. In some embodiments, a Defense System disclosed herein provides a bacterial cell with resistance to a foreign nucleic acid invasion. In some embodiments, a Defense System disclosed herein provides a bacterial cell with protection to foreign nucleic acid invasion.

In some embodiments, a method disclosed herein protects a host cell from foreign nucleic acid invasion, said method comprising a step of introducing into the host cell at least one defense system described herein. In some embodiments, a method disclosed herein protects a host cell from foreign nucleic acid invasion, said method comprising a step of introducing into the host cell at least one function defense system described herein. In some embodiments, a method disclosed herein protects a bacterial cell from foreign nucleic acid invasion, said method comprising a step of introducing into the bacterial cell at least one defense system described herein. In some embodiments, a method disclosed herein protects a bacterial cell from foreign nucleic acid invasion, said method comprising a step of introducing into the bacterial cell at least one function defense system described herein.

In some embodiments, protecting a host cell from foreign nucleic acid invasion comprises protecting from phage infection, protecting from plasmid transformation, or protection from conjugative element entry, or any combination thereof. In some embodiments, protecting a bacterial cell from foreign nucleic acid invasion comprises protecting from phage infection, protecting from plasmid transformation, or protection from conjugative element entry, or any combination thereof. In some embodiments, protection from phage infection comprises protection from at least one phage infection.

In some embodiments, a nucleic acid construct comprising any one of the defense systems described herein or any combination thereof, provides a host cell with resistance to foreign nucleic acid invasion. In some embodiments, a nucleic acid construct comprising any one of the defense systems described herein or any combination thereof, provides protection from foreign nucleic acid invasion to a host cells.

In some embodiments, a nucleic acid construct comprising any one of the defense systems described herein or any combination thereof, provides a bacterial cell with resistance to foreign nucleic acid invasion. In some embodiments, a nucleic acid construct comprising any one of the defense systems described herein or any combination thereof, provides protection from foreign nucleic acid invasion to a bacterial cells.

In some embodiments, a Defense System disclosed herein provides a host cell with resistance to at least one phage. In some embodiments, a Defense System disclosed herein provides a host cell with resistance to a plasmid. In some embodiments, a Defense System disclosed herein provides a host cell with resistance to a combination of at least one phage and a plasmid.

In some embodiments, a Defense System selected from a Defense System Ia, Ib, II, IIIa, IIIb, IIIc, IV, V, VI, VII, VIII, IX, Xa, Xb, and Xc, provides a host cell with resistance to at least one phage. In some embodiments, a Defense selected from a Defense System Ia, Ib, II, IIIa, IIIb, IIIc, IV, V, VI, VII, VIII, IX, Xa, Xb, and Xc, provides a host cell with resistance to a plasmid. In some embodiments, a Defense System selected from a Defense System Ia, Ib, II, IIIa, IIIb, IIIc, IV, V, VI, VII, VIII, IX, Xa, Xb, and Xc, provides a host cell with resistance to a combination of at least one phage and a plasmid.

In some embodiments, a defense system comprising a combination of Defense Systems selected from Defense Systems Ia, Ib, II, IIIa, IIIb, IIIc, IV, V, VI, VII, VIII, IX, Xa, Xb, and Xc, provides a host cell with resistance to at least one phage. In some embodiments, a defense system comprising a combination of Defense Systems selected from Defense Systems Ia, Ib, II, IIIa, IIIb, IIIc, IV, V, VI, VII, VIII, IX, Xa, Xb, and Xc, provides a host cell with resistance to a plasmid. In some embodiments, a defense system comprising a combination of Defense Systems selected from Defense Systems Ia, Ib, II, IIIa, IIIb, IIIc, IV, V, VI, VII, VIII, IX, Xa, Xb, and Xc, provides a host cell with resistance to a combination of at least one phage and a plasmid.

In some embodiments, resistance is to at least one phage. In some embodiments, resistance to at least one phage comprises resistance to more than one phage. In some embodiments, resistance is to phage comprises resistance to phages from different families (See for example FIGS. 3B and 3C, and FIG. 5D).

A skilled artisan would appreciate that the term “phage” or “bacteriophage” refers to a virus that selectively infects one or more bacterial species. Many phages are specific to a particular genus or species or strain of bacteria. In some embodiments, a phage or a bacteriophage selectively infects one or more archaeal species. Many phages are specific to a particular genus or species or strain of archaea.

In some embodiments, the phage genome can be ssDNA, dsDNA, ssRNA, or dsRNA. In some embodiments, the phage genome is linear. In some embodiments, the phage genome is circular. In some embodiments, the phage is virulent to bacteria. In some embodiments, the phage is virulent to archaea. In some embodiments, the phage is virulent to bacteria and archaea.

In some embodiments, the phage is a lytic phage. In some embodiments, the phage is temperate (also referred to as lysogenic). In some embodiments, the phage is a lytic phage selected from the group consisting of Nf, SP82G and SPO1. In some embodiments, the phage is Nf. In some embodiments, the phage is SPO1. In some embodiments, the phage is a temperate phage selected from the group consisting of phi3T and phi105. In some embodiments, the phage is phi3T.

In some embodiments, phage as disclosed herein include, but are not limited to, phages that belong to any of the following virus families: Caudavirales, Corticoviridae, Cystoviridae, Inoviridae, Leviviridae, Microviridae, Myoviridae, Podoviridae, Siphoviridae, or Tectiviridae. In some embodiments, the phage is a podovirus, a myovirus or a siphovirus phage. In some embodiments, the phage is a T7 or a lambda-vir phage. In some embodiments, a phage comprises phage that infect bacteria that are pathogenic to plants and/or animals (including humans).

In some embodiments, the resistance of a host cell comprising a defense system against a phage is improved as compared to a host cell of the same species, which does not include a defense system disclosed herein.

Assays for testing phage resistance are well known in the art. Thus, for example, the lysogenic activity of a phage can be assessed by PCR or DNA sequencing. The DNA replication activity of a phage can be assessed e.g. by DNA sequencing or Southern blot analysis.

The lytic activity of a phage can be assessed e.g. by optical density, plaque assay or living dye indicators. The lytic activity of a phage can be measured indirectly by following the decrease in optical density of the bacterial cultures owing to lysis. This method involves introduction of phage into a fluid bacterial culture medium. After a period of incubation, the phage lyses the bacteria in the broth culture resulting in a clearing of the fluid medium resulting in decrease in optical density.

Another method, known as the plaque assay, introduces phage into a few milliliters of soft agar along with some bacterial host cells. This soft agar mixture is laid over a hard agar base (seeded-agar overlay). The phage adsorbs onto the host bacterial cells, infect and lyse the cells, and then begin the process anew with other bacterial cells in the vicinity. After 6-24 hours, zones of clearing on the plate, known as plaques, are observable within the lawn of bacterial growth on the plate. Each plaque represents a single infective phage particle in the original sample.

Yet another method is the one-step phage growth curve which allows determining the production of progeny virions by cells as a function of time after infection. The assay is based on the fact that cells in the culture are infected simultaneously with a low number of phages so that no cell can be infected with more than one phage. At various time intervals, samples are removed for a plaque assay allowing quantitative determination of the number of phages present in the medium.

Other methods use for example redox chemistry, employing cell respiration as a universal reporter. During active growth of bacteria, cellular respiration reduces a dye (e.g., tetrazolium dye) and produces a color change that can be measured in an automated fashion. On the other hand, successful phage infection and subsequent growth of the phage in its host bacterium results in reduced bacterial growth and respiration and a concomitant reduction in color.

Thus, nucleic acid sequences encoding a Defense System component(s) or functional fragments thereof, and the polypeptides and nucleic acid constructs disclosed herein can be used in conferring phage resistance thereby protecting bacteria from phage infection.

In some embodiments, “conferring phage resistance” refers to the level of phage infection and/or multiplication in the e.g. bacteria containing a functional Defense System as disclosed herein, that does not cause a deleterious effect to the bacteria e.g., growth arrest or death.

In some embodiments, the phage has about 10-10,000 times lower efficiency of plaquing on bacteria containing a functional Defense System. In some embodiments, the phage has about 100-100,000 times lower efficiency of plaquing on bacteria containing a functional Defense System ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a functional Defense System Ia ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a functional Defense System Ia ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a functional Defense System Ia ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a functional Defense System Ib ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a functional Defense System II ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a functional Defense System III (Type I, Type II, and Type III) ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a functional Defense System IV ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a functional Defense System V ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a functional Defense System VI ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a functional Defense System VII ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a functional Defense System VIII ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a functional Defense System IX ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a functional Defense System Xa ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a functional Defense System Xb ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a functional Defense System Xc ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours.

In some embodiments, the phage has about 100-100,000 times lower efficiency of plaquing on bacteria containing a combination of functional Defense Systems ([EOP]=10⁻²), about 1000 times lower EOP (EOP=10⁻³), 10,000 times lower EOP (EOP=10⁻⁴), or 100,000 times lower EOP (EOP=10⁻⁵). In some embodiments, the level of phage multiplication in a culture is measured after about 6-14 hours incubation of the culture, e.g., after about 12 hours, after about 9 hours, after about 8 hours after about 7 hours, or after about 6 hours. In some embodiments, the combination of Defense Systems comprises any combination of Defense Systems Ia, Ib, II, IIIa, IIIb, IIIc, IV, V, VI, VII, VIII, IX, Xa, Xb, and Xc as described herein.

In some embodiments, a Defense System provides resistance is to a plasmid. In some embodiments, the resistance of a host cell comprising a defense system against a phage is improved as compared to a host cell of the same species, which does not include a defense system disclosed herein. In some embodiments, a plasmid comprises an episomal plasmid. In some embodiments, a plasmid comprises an integrative plasmid. In some embodiments, resistance to a plasmid comprises decrease transformation efficiency of the plasmid. In some embodiments, resistance to a plasmid comprises decrease transformation efficiency of the episomal plasmid.

In some embodiments, a nucleic acid sequences encoding a Defense System component(s) or functional fragments thereof, and the polypeptides and nucleic acid constructs disclosed herein can be used in protecting bacteria from conjugative elements. In some embodiments, disclosed herein is a method of protecting bacteria from conjugative elements, the method comprising the step of introducing into the bacteria a defense system selected from Defense Systems Ia-Xc, or a combination thereof. In some embodiments, disclosed herein is a method of protecting bacteria from conjugative elements, the method comprising the step of introducing into the bacteria a functional defense system selected from Defense Systems Ia-Xc, or a combination thereof. In some embodiments, disclosed herein is a method of protecting bacteria from conjugative elements, the method comprising the step of introducing into the bacteria at least one component of a defense systems selected from Defense Systems Ia-Xc, or a combination thereof.

A skilled artisan would appreciate that a method protecting bacteria from conjugative elements, would in some embodiments, encompass a reduction in the horizontal gene transfer between bacteria. The steps of conjugation known in the art comprise an initial step of cell-to-cell contact during mating pair formation, followed by a transfer of gene(s)/genetic elements in three steps. The DNA is nicked at the origin of transfer, wherein the nicking enzyme binds covalently to one of the DNA strands. The nucleoprotein filament is then coupled to the type 4 secretion system (T4SS) and transferred to the recipient cell. Finally, the element is replicated in the original and novel hosts leading to double stranded DNA molecules in each cell. (Cury et al. (2017) Nucleic Acids Res. September 6; 45(15): 8943-8956).

In some embodiments, a method of protecting bacteria from conjugative elements inhibits or reduces cell-to-cell contact or mating pair formation. In some embodiments, a method of protecting bacteria from conjugative elements inhibits or reduces nicking of the DNA strand. In some embodiments, a method of protecting bacteria from conjugative elements inhibits or reduces transfer of the DNA into the recipient cell. In some embodiments, a method of protecting bacteria from conjugative elements inhibits or reduces replication of the element in the novel host comprising a defense system described herein, a combination thereof, of at least one element thereof. In some embodiments, the conjugative element comprises a plasmid. In some embodiments, the conjugative element comprises a transposon.

In some embodiments, there is provided an article of manufacture or a kit identified for providing bacterial resistance, or plasmid transformation, or bacterial resistance and plasmid transformation to a microbial cell, for example but not limited to bacteria, comprising a defense system disclosed herein. In some embodiments, the article of manufacture or kit is for use producing bacteria resistant to at least one phage, wherein the bacteria do not naturally comprise the defense system. In some embodiments, the article of manufacture or kit is for use producing bacteria resistant to plasmid transformation, wherein the bacteria do not naturally comprise the defense system. In some embodiments, the article of manufacture or kit is for use producing bacteria resistant to at least one phage and resistant to plasmid transformation, wherein the bacteria do not naturally comprise the defense system.

In some embodiments, the defense system comprises at least 2 Defense Systems, wherein the bacteria do not naturally comprise the at least 2 Defense Systems. In some embodiments, the defense system comprises any of Defense Systems Ia-Xc or a combination thereof, wherein the bacteria do not naturally comprise the Defense System or combination of systems. In some embodiments, the defense system comprises a combination of Defense systems selected from Defense Systems Ia-Xc, wherein the bacteria do not naturally comprise the combination of Defense Systems present in the article of manufacture or the kit.

In some embodiments, a Defense System described herein is packaged in a container. In some embodiments, different Defense Systems are packaged in separate containers. In some embodiments, different Defense Systems are packaged in the same containers. In some embodiments, 2, 3, 4, 5, 6, 7, 8, 9, 10, 11, 12, 13, 14, or 15 different Defense Systems are packaged in the same containers. In some embodiments, the different Defense Systems are in a co-formulation. In some embodiments, different Defense Systems are packaged in transportable containers.

In some embodiments, a container comprising a Defense Systems described herein comprises lyophilized components of said Defense System. In some embodiments, a container comprising at least two Defense Systems described herein comprises lyophilized components of said Defense Systems. In some embodiments, a container comprising at least 2, 3, 4, 5, 6, 7, 8, 9, 10, 11, 12, 13, 14, or 15 Defense Systems described herein comprises lyophilized components of said Defense Systems. In some embodiments, Defense Systems components are co-formulated and lyophilized and stored in a container.

In some embodiments, a container comprising a Defense Systems described herein comprises spray-dried components of said Defense System. In some embodiments, a container comprising at least two Defense Systems described herein comprises spray-dried components of said Defense Systems. In some embodiments, a container comprising at least 2, 3, 4, 5, 6, 7, 8, 9, 10, 11, 12, 13, 14, or 15 Defense Systems described herein comprises spray-dried components of said Defense Systems. In some embodiments, Defense Systems components are co-formulated and spray-dried and stored in a container.

In some embodiments, a container comprising a Defense Systems described herein comprises components of said Defense System in a liquid formulation. In some embodiments, a container comprising at least two Defense Systems described herein comprises components of said Defense Systems in a liquid formulation. In some embodiments, a container comprising at least 2, 3, 4, 5, 6, 7, 8, 9, 10, 11, 12, 13, 14, or 15 Defense Systems described herein comprises components of said Defense Systems in a liquid formulation. In some embodiments, Defense Systems components are co-formulated and stored in a container in a liquid formulation.

In some embodiments, a Defense system stored in a container comprises a full complement of components. In some embodiments, a Defense system stored in a container comprises less than a full complement of components. In some embodiments, a Defense system stored in a container comprises at least one component of the Defense system. In some embodiments, a Defense system stored in a container comprises at least 1, 2, 3, 4, or 5 component of the Defense system. In some embodiments, a Defense system stored in a container comprises between about 1-10 component of the Defense system. In some embodiments, a Defense system stored in a container comprises more than components of the Defense system. In some embodiments, a Defense system stored in a container comprises different numbers of components of different Defense systems. For example but not limited to, in some embodiments a container comprises a full complement of components of one Defense System and less than a full complement of components of another Defense system.

### Methods of Use of a Defense System or Combinations Thereof

Defense systems have been described in detail above, as has the combination of Defense Systems each comprising a nucleic acid construct. In some embodiments, methods of use a defense system disclosed herein comprises use of a single Defense System comprising a nucleic acid construct, selected from Defense Systems Ia, Ib, II, IIIa, IIIa, IIIa, IV, V, VI, VII, VIII, IX, Xa, Xb, and Xc. In some embodiments, methods of use of a defense system comprises use of a combination of Defense Systems comprising nucleic acid constructs selected from Defense systems Ia, Ib, II, IIIa, IIIa, IIIa, IV, V, VI, VII, VIII, IX, Xa, Xb, and Xc, as disclosed herein.

Methods of use of a defense system described herein include but are not limited to, methods of protecting bacteria from phage infection, methods of protecting bacteria from plasmid transformation, methods of protecting bacteria from conjugative elements, methods of protecting bacteria from any combination of phage infection, conjugative elements, and plasmid transformation, methods of producing bacteria resistant to phage infection; methods of producing bacteria resistant to plasmids; methods of producing bacteria protected from conjugative elements, methods of producing bacteria resistant to phage infection, conjugative elements, or plasmids, or any combination thereof; and methods of making a food, a food additive, a feed, a nutritional supplement, a probiotic supplement, a personal care product, a health care product, and/or a veterinary product.

In some embodiments, a method of use described herein comprises a method of protecting a host cell from foreign nucleic acid invasion. A method of use described herein comprises a method of protecting a bacterial cell from foreign nucleic acid invasion.

In some embodiments, a method of protecting a host cell from foreign nucleic acid invasion comprises introducing into the host cells at least one defense system described herein. In some embodiments, a method of protecting a host cell from foreign nucleic acid invasion comprises introducing into the host cells a combination of defense systems described herein. In some embodiments, a method of protecting a host cell from foreign nucleic acid invasion comprises introducing into the host cells at least 1, 2, 3, 4, 5, 6, 7, 8, 9, 10, 11, 12, 13, 14, or 15 defense system described herein.

In some embodiments, a method of protecting a host cell from foreign nucleic acid invasion comprises introducing into the host cells at least one functional defense system described herein. In some embodiments, a method of protecting a host cell from foreign nucleic acid invasion comprises introducing into the host cells a combination of functional defense systems described herein. In some embodiments, a method of protecting a host cell from foreign nucleic acid invasion comprises introducing into the host cells at least 1, 2, 3, 4, 5, 6, 7, 8, 9, 10, 11, 12, 13, 14, or 15 functional defense system described herein. In some embodiments, a method of protecting a host cell from foreign nucleic acid invasion comprises introducing into the host cells at least 1, 2, 3, 4, 5, 6, 7, 8, 9, 10, 11, 12, 13, 14, or 15 functional and non-functional defense system described herein.

In some embodiments, a method of protecting a bacterial cell from foreign nucleic acid invasion comprises introducing into the bacterial cells at least one defense system described herein. In some embodiments, a method of protecting a bacterial cell from foreign nucleic acid invasion comprises introducing into the bacterial cells a combination of defense systems described herein. In some embodiments, a method of protecting a bacterial cell from foreign nucleic acid invasion comprises introducing into the bacterial cells at least 1, 2, 3, 4, 5, 6, 7, 8, 9, 10, 11, 12, 13, 14, or 15 defense system described herein.

In some embodiments, a method of protecting a bacterial cell from foreign nucleic acid invasion comprises introducing into the bacterial cells at least one functional defense system described herein. In some embodiments, a method of protecting a bacterial cell from foreign nucleic acid invasion comprises introducing into the bacterial cells a combination of functional defense systems described herein. In some embodiments, a method of protecting a bacterial cell from foreign nucleic acid invasion comprises introducing into the bacterial cells at least 1, 2, 3, 4, 5, 6, 7, 8, 9, 10, 11, 12, 13, 14, or 15 functional defense system described herein. In some embodiments, a method of protecting a bacterial cell from foreign nucleic acid invasion comprises introducing into the bacterial cells at least 1, 2, 3, 4, 5, 6, 7, 8, 9, 10, 11, 12, 13, 14, or 15 functional and non-functional defense system described herein.

In some embodiments, a method of protecting a host cell from foreign nucleic acid invasion comprises protecting said host cell from phage infection. In some embodiments, a method of protecting a host cell from foreign nucleic acid invasion comprises protecting said host cell from at least one phage infection. In some embodiments, a method of protecting a host cell from foreign nucleic acid invasion comprises protecting said host cell from more than one phage infection. In some embodiments, a method of protecting a host cell from foreign nucleic acid invasion comprises protecting said host cell from plasmid transformation. In some embodiments, a method of protecting a host cell from foreign nucleic acid invasion comprises protecting said host cell from at least one plasmid transformation. In some embodiments, a method of protecting a host cell from foreign nucleic acid invasion comprises protecting said host cell from more than one plasmid transformation. In some embodiments, a method of protecting a host cell from foreign nucleic acid invasion comprises protecting said host cell from entry of conjugative elements. In some embodiments, a method of protecting a host cell from foreign nucleic acid invasion comprises protecting said host cell from entry of at least one conjugative element. In some embodiments, a method of protecting a host cell from foreign nucleic acid invasion comprises protecting said host cell from entry of more than one conjugative elements. In some embodiments, a method of protecting a host cell from foreign nucleic acid invasion comprises protecting said host cell from any combination of phage infection, plasmid transformation, and entry of conjugative elements.

In some embodiments, a method of protecting a bacterial cell from foreign nucleic acid invasion comprises protecting said bacterial cell from phage infection. In some embodiments, a method of protecting a bacterial cell from foreign nucleic acid invasion comprises protecting said bacterial cell from at least one phage infection. In some embodiments, a method of protecting a bacterial cell from foreign nucleic acid invasion comprises protecting said bacterial cell from more than one phage infection. In some embodiments, a method of protecting a bacterial cell from foreign nucleic acid invasion comprises protecting said bacterial cell from plasmid transformation. In some embodiments, a method of protecting a bacterial cell from foreign nucleic acid invasion comprises protecting said bacterial cell from at least one plasmid transformation. In some embodiments, a method of protecting a bacterial cell from foreign nucleic acid invasion comprises protecting said bacterial cell from more than one plasmid transformation. In some embodiments, a method of protecting a bacterial cell from foreign nucleic acid invasion comprises protecting said bacterial cell from entry of conjugative elements. In some embodiments, a method of protecting a bacterial cell from foreign nucleic acid invasion comprises protecting said bacterial cell from entry of at least one conjugative element. In some embodiments, a method of protecting a bacterial cell from foreign nucleic acid invasion comprises protecting said bacterial cell from entry of more than one conjugative elements. In some embodiments, a method of protecting a bacterial cell from foreign nucleic acid invasion comprises protecting said bacterial cell from any combination of phage infection, plasmid transformation, and entry of conjugative elements.

In some embodiments, a method disclosed herein comprises the use of a defense system described herein, for protecting bacteria from phage infection. In some embodiments, a method disclosed herein comprises a method of protecting bacteria from plasmid transformation. In some embodiments, a method disclosed herein comprises a method of protecting bacteria from conjugative elements. In some embodiments, a method disclosed herein comprises a method of protecting bacteria from phage infection, and/or conjugative elements, and/or plasmid transformation. In some embodiments, a method disclosed herein comprises a method of producing bacteria resistant to phage infection. In some embodiments, a method disclosed herein comprises a method of producing bacteria resistant to plasmids. In some embodiments, a method disclosed herein comprises a method of producing bacteria resistant to conjugative elements. In some embodiments, a method disclosed herein comprises a method of producing bacteria resistant to phage infection, and/o conjugative elements, and/or plasmids.

In some embodiments, a method disclosed herein comprises a method of making a food. In some embodiments, a method disclosed herein comprises a method of making a food additive. In some embodiments, a method disclosed herein comprises a method of making a feed. In some embodiments, a method disclosed herein comprises a method of making a nutritional supplement. In some embodiments, a method disclosed herein comprises a method of making a probiotic supplement. In some embodiments, a method disclosed herein comprises a method of making a personal care product. In some embodiments, a method disclosed herein comprises a method of making a health care product. In some embodiments, a method disclosed herein comprises a method of making a veterinary product.

In some embodiments, disclosed herein is a method of protecting bacteria from phage infection, said method comprising the step of introducing into bacteria a defense system comprising at least one of a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, or a Defense System Xc, wherein each nucleic acid construct of each Defense System Ia-Xc further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof; wherein said method results in the bacteria being resistant to at least one phage, or being resistant to a plasmid, or being resistant to at least one phage and a plasmid.

In some embodiments, disclosed herein is a method of protecting bacteria from plasmid transformation, said method comprising the step of introducing into bacteria a defense system comprising at least one of a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, or a Defense System Xc, wherein each nucleic acid construct of each Defense System Ia-Xc further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof; wherein said method results in the bacteria being resistant to at least one phage, or being resistant to a plasmid, or being resistant to at least one phage and a plasmid.

In some embodiments, disclosed herein is a method of protecting bacteria from conjugative elements, said method comprising the step of introducing into bacteria a defense system comprising at least one of a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, or a Defense System Xc, wherein each nucleic acid construct of each Defense System Ia-Xc further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof; wherein said method results in the bacteria being resistant to at least one conjugative elements.

In some embodiments, disclosed herein is a method of protecting bacteria from any combination of phage infection, conjugative elements, and plasmid transformation, said method comprising the step of introducing into bacteria a defense system comprising at least one of a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, or a Defense System Xc, wherein each nucleic acid construct of each Defense System Ia-Xc further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof; wherein said method results in the bacteria being resistant to at least one phage, or being resistant to a plasmid, or being resistant to a conjugative element, or being resistant to a combination of at least one phage, a conjugative element, and a plasmid.

In some embodiments, disclosed herein is a method of producing bacteria resistant to phage infection, said method comprising the step of introducing into bacteria a defense system comprising at least one of a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, or a Defense System Xc, wherein each nucleic acid construct of each Defense System Ia-Xc further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof; wherein said method produces bacteria resistant to at least one phage.

In some embodiments, disclosed herein is a method of producing bacteria resistant to plasmid transformation, said method comprising the step of introducing into bacteria a defense system comprising at least one of a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, or a Defense System Xc, wherein each nucleic acid construct of each Defense System Ia-Xc further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof; wherein said method produces bacteria resistant to a plasmid transformation.

In some embodiments, disclosed herein is a method of producing bacteria resistant to conjugative elements, said method comprising the step of introducing into bacteria a defense system comprising at least one of a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, or a Defense System Xc, wherein each nucleic acid construct of each Defense System Ia-Xc further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof; wherein said method produces bacteria resistant to conjugative elements.

In some embodiments, disclosed herein is a method of producing bacteria resistant to any combination of phage infection, conjugative elements, and plasmid transformation, said method comprising the step of introducing into bacteria a defense system comprising at least one of a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, or a Defense System Xc, wherein each nucleic acid construct of each Defense System Ia-Xc further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof; wherein said method produces bacteria resistant to any combination of phage infection, conjugative elements, and plasmid transformation.

In some embodiments, the defense system used in a method disclosed herein comprises one of a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, and a Defense System Xc. In some embodiments, the defense system used in a method disclosed herein comprises a combination of Defense Systems selected from a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, and a Defense System Xc. In some embodiments, the defense system used in a method disclosed herein comprises between 2-5 Defense Systems selected from a Defense System Ia, a Defense System Ib, a Defense System II, a Defense System III, a Defense System IV, a Defense System V, a Defense System VI, a Defense System VII, a Defense System VIII, a Defense System IX, a Defense System Xa, a Defense System Xb, and a Defense System Xc. In some embodiments, the defense system used in a method disclosed herein comprises between 2-10 Defense Systems selected from a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, and a Defense System Xc. In some embodiments, the defense system used in a method disclosed herein comprises between 10-20 Defense Systems selected from a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, and a Defense System Xc.

In some embodiments, the defense system used in a method disclosed herein comprises a combination of Defense Systems, as described in detail herein, selected from a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, and a Defense System Xc.

In some embodiments, disclosed herein is a method of protecting bacteria from phage infection, said method comprising the step of introducing into bacteria a defense systems comprising a combination of Defense Systems, said combination comprising any combination of a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, or a Defense System Xc, wherein each nucleic acid construct of each Defense System Ia-Xc further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof; wherein said method results in the bacteria being resistant to at least one phage.

In some embodiments, disclosed herein is a method of protecting bacteria from plasmid transformation, said method comprising the step of introducing into bacteria a defense systems comprising a combination of Defense Systems, said combination comprising any combination of a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, or a Defense System Xc, wherein each nucleic acid construct of each Defense System Ia-Xc further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof; wherein said method results in the bacteria being resistant to a plasmid transformation.

In some embodiments, disclosed herein is a method of protecting bacteria from conjugative elements, said method comprising the step of introducing into bacteria a defense systems comprising a combination of Defense Systems, said combination comprising any combination of a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, or a Defense System Xc, wherein each nucleic acid construct of each Defense System Ia-Xc further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof; wherein said method results in the bacteria being resistant to conjugative elements.

In some embodiments, disclosed herein is a method of protecting bacteria from any combination of phage infection, conjugative elements, and plasmid transformation, said method comprising the step of introducing into bacteria a defense systems comprising a combination of Defense Systems, said combination comprising any combination of a Defense System Ia, a Defense System Ib, a Defense System II, a Defense System IIIa, a Defense System IIIb, a Defense System IIIc, a Defense System IV, a Defense System V, a Defense System VI, a Defense System VII, a Defense System VIII, a Defense System IX, a Defense System Xa, a Defense System Xb, or a Defense System Xc, wherein each nucleic acid construct of each Defense System Ia-Xc further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof; wherein said method results in the bacteria being resistant to any combination of phage infection, conjugative elements, and plasmid transformation.

In some embodiments, a method of protecting bacteria from phage infection produces bacteria resistant to phage infection. In some embodiments, the method of protecting bacteria from plasmid transformation produces bacteria having reduced transformation efficiency of a plasmid. In some embodiments, the method of protecting bacteria from conjugative elements produces bacteria resistant to horizontal gene transfer. In some embodiments, the method of protecting bacteria from phage infection, and/or conjugative elements, and/or plasmid transformation, produces bacteria resistant to phage infection, resistant to horizontal gene transfer, and having reduced transformation efficiency of a plasmid.

In some embodiments, a method of protecting bacteria from phage infection produces bacteria resistant to at least one phage. In some embodiments, a method of protecting bacteria from plasmid transformation produces bacteria resistant to plasmid transformation. In some embodiments, a method of protecting bacteria from phage infection and plasmid transformation produces bacteria resistant to at least one phage and resistant to entry of a plasmid.

In some embodiments, provided herein is a method of protecting bacteria from phage infection, the method comprising introducing into the bacteria a defense system having an anti-phage activity, the system comprising at least one component selected from the group consisting of the components of the defense system, thereby protecting the bacteria from phage infection. In some embodiments, a method of protecting bacteria from phage infection comprises introducing into the bacteria a defense system comprising a Defense System selected from a Defense System Ia, Ib, II, IIIa, IIIb, IIIc, IV, V, VI, VII, VIII, IX, Xa, Xb, and Xc, wherein said defense system comprises an anti-phage activity, the system comprising at least one component of said Defense System Ia, Ib, II, IIIa, IIIb, IIIc, IV, V, VI, VII, VIII, IX, Xa, Xb, or Xc, said at least one component selected from the group consisting of the components of the Defense Systems Ia, Ib, II, IIIa, IIIb, IIIc, IV, V, VI, VII, VIII, IX, Xa, Xb, or Xc, respectively, thereby protecting the bacteria from phage infection. In some embodiments, a method of protecting bacteria from phage infection comprises introducing into the bacteria a defense system comprising a combination of Defense Systems selected from Defense System Ia, Ib, II, IIIa, IIIb, IIIc, IV, V, VI, VII, VIII, IX, Xa, Xb, and Xc, wherein said defense system comprises an anti-phage activity, the system comprising at least one component of each of said Defense Systems in combination, said at least one component selected from the group consisting of the components of the Defense Systems Ia, Ib, II, IIIa, IIIb, IIIc, IV, V, VI, VII, VIII, IX, Xa, Xb, or Xc, respectively, thereby protecting the bacteria from phage infection.

In some embodiments, an at least one component comprises the components of a Defense System Ia, Ib, II, IIIa, IIIb, IIIc, IV, V, VI, VII, VIII, IX, Xa, Xb, or Xc, required for a functional Defense System, wherein said function comprises an anti-phage activity. In some embodiments, an at least one component comprises the components of the cassette of genes making up a Defense System Ia, Ib, II, IIIa, IIIb, IIIc, IV, V, VI, VII, VIII, IX, Xa, Xb, or Xc. In some embodiments, an at least one component comprises two components of a Defense System Ia, Ib, II, IIIa, IIIb, IIIc, IV, V, VI, VII, VIII, IX, Xa, Xb, or Xc. In some embodiments, an at least one component comprises three components of a Defense System Ia, Ib, II, IIIa, IIIb, IIIc, IV, V, VI, VII, VIII, IX, Xa, Xb, or Xc. In some embodiments, an at least one component comprises four components of a Defense System Ia, Ib, II, IIIa, IIIb, IIIc, IV, V, VI, VII, VIII, IX, Xa, Xb, or Xc. In some embodiments, an at least one component comprises five components of a Defense System Ia, Ib, II, IIIa, IIIb, IIIc, IV, V, VI, VII, VIII, IX, Xa, Xb, or Xc. In some embodiments, a component comprises the full-length component. In some embodiments, a component comprises a functional portion of the component. In some embodiments, a component comprises a component homolog.

In some embodiments, provided herein is a method of protecting bacteria from plasmid transformation, the method comprising introducing into the bacteria a defense system having an anti-plasmid activity, the system comprising at least one component selected from the group consisting of the components of the defense system, thereby protecting the bacteria from plasmid transformation. In some embodiments, a method of protecting bacteria from plasmid transformation comprises introducing into the bacteria a defense system comprising a Defense System selected from a Defense System Ia, Ib, II, IIIa, IIIb, IIIc, IV, V, VI, VII, VIII, IX, Xa, Xb, and Xc, wherein said defense system comprises an anti-plasmid activity, the system comprising at least one component of said Defense System Ia, Ib, II, IIIa, IIIb, IIIc, IV, V, VI, VII, VIII, IX, Xa, Xb, or Xc, said at least one component selected from the group consisting of the components of the Defense Systems Ia, Ib, II, IIIa, IIIb, IIIc, IV, V, VI, VII, VIII, IX, Xa, Xb, or Xc, respectively, thereby protecting the bacteria from plasmid transformation. In some embodiments, a method of protecting bacteria from plasmid transformation comprises introducing into the bacteria a defense system comprising a combination of Defense Systems selected from Defense System Ia, Ib, II, IIIa, IIIb, IIIc, IV, V, VI, VII, VIII, IX, Xa, Xb, and Xc, wherein said defense system comprises an anti-plasmid activity, the system comprising at least one component of each of said Defense Systems in combination, said at least one component selected from the group consisting of the components of the Defense Systems Ia, Ib, II, IIIa, IIIb, IIIc, IV, V, VI, VII, VIII, IX, Xa, Xb, or Xc, respectively, thereby protecting the bacteria from plasmid transformation.

In some embodiments, an at least one component comprises the components of a Defense System Ia, Ib, II, IIIa, IIIb, IIIc, IV, V, VI, VII, VIII, IX, Xa, Xb, or Xc, required for a functional Defense System, wherein said function comprises an anti-plasmid activity. In some embodiments, an at least one component comprises the components of the cassette of genes making up a Defense System Ia, Ib, II, IIIa, IIIb, IIIc, IV, V, VI, VII, VIII, IX, Xa, Xb, or Xc. In some embodiments, an at least one component comprises two components of a Defense System Ia, Ib, II, IIIa, IIIb, IIIc, IV, V, VI, VII, VIII, IX, Xa, Xb, or Xc. In some embodiments, an at least one component comprises three components of a Defense System Ia, Ib, II, IIIa, IIIb, IIIc, IV, V, VI, VII, VIII, IX, Xa, Xb, or Xc. In some embodiments, an at least one component comprises four components of a Defense System Ia, Ib, II, IIIa, IIIb, IIIc, IV, V, VI, VII, VIII, IX, Xa, Xb, or Xc. In some embodiments, an at least one component comprises five components of a Defense System Ia, Ib, II, IIIa, IIIb, IIIc, IV, V, VI, VII, VIII, IX, Xa, Xb, or Xc. In some embodiments, a component comprises the full-length component. In some embodiments, a component comprises a functional portion of the component. In some embodiments, a component comprises a component homolog.

In some embodiments, provided herein is a method of protecting bacteria from conjugative elements, the method comprising introducing into the bacteria a defense system having an anti-conjugation activity, the system comprising at least one component selected from the group consisting of the components of the defense system, thereby protecting the bacteria from conjugation. In some embodiments, a method of protecting bacteria from conjugative elements comprises introducing into the bacteria a defense system comprising a Defense System selected from a Defense System Ia, Ib, II, IIIa, IIIb, IIIc, IV, V, VI, VII, VIII, IX, Xa, Xb, and Xc, wherein said defense system comprises an anti-conjugation activity, the system comprising at least one component of said Defense System Ia, Ib, II, IIIa, IIIb, IIIc, IV, V, VI, VII, VIII, IX, Xa, Xb, or Xc, said at least one component selected from the group consisting of the components of the Defense Systems Ia, Ib, II, IIIa, IIIb, IIIc, IV, V, VI, VII, VIII, IX, Xa, Xb, or Xc, respectively, thereby protecting the bacteria from conjugation. In some embodiments, a method of protecting bacteria from conjugative elements comprises introducing into the bacteria a defense system comprising a combination of Defense Systems selected from Defense System Ia, Ib, II, IIIa, IIIb, IIIc, IV, V, VI, VII, VIII, IX, Xa, Xb, and Xc, wherein said defense system comprises an anti-conjugation activity, the system comprising at least one component of each of said Defense Systems in combination, said at least one component selected from the group consisting of the components of the Defense Systems Ia, Ib, II, IIIa, IIIb, IIIc, IV, V, VI, VII, VIII, IX, Xa, Xb, or Xc, respectively, thereby protecting the bacteria from conjugation.

In some embodiments, an at least one component comprises the components of a Defense System Ia, Ib, II, IIIa, IIIb, IIIc, IV, V, VI, VII, VIII, IX, Xa, Xb, or Xc, required for a functional Defense System, wherein said function comprises an anti-conjugation activity. In some embodiments, an at least one component comprises the components of the cassette of genes making up a Defense System Ia, Ib, II, IIIa, IIIb, IIIc, IV, V, VI, VII, VIII, IX, Xa, Xb, or Xc. In some embodiments, an at least one component comprises two components of a Defense System Ia, Ib, II, IIIa, IIIb, IIIc, IV, V, VI, VII, VIII, IX, Xa, Xb, or Xc. In some embodiments, an at least one component comprises three components of a Defense System Ia, Ib, II, IIIa, IIIb, IIIc, IV, V, VI, VII, VIII, IX, Xa, Xb, or Xc. In some embodiments, an at least one component comprises four components of a Defense System Ia, Ib, II, IIIa, IIIb, IIIc, IV, V, VI, VII, VIII, IX, Xa, Xb, or Xc. In some embodiments, an at least one component comprises five components of a Defense System Ia, Ib, II, IIIa, IIIb, IIIc, IV, V, VI, VII, VIII, IX, Xa, Xb, or Xc. In some embodiments, a component comprises the full-length component. In some embodiments, a component comprises a functional portion of the component. In some embodiments, a component comprises a component homolog.

In some embodiments, provided herein is a method of protecting bacteria from any combination of phage infection, conjugative elements, and plasmid transformation, the method comprising introducing into the bacteria a defense system having an anti-phage, anti-conjugation, and/or anti-plasmid activity, the system comprising at least one component selected from the group consisting of the components of the defense system, thereby protecting the bacteria from phage infection, conjugative elements, and plasmid transformation. In some embodiments, a method of protecting bacteria from phage infection, conjugative elements, and plasmid transformation comprises introducing into the bacteria a defense system comprising a Defense System selected from a Defense System Ia, Ib, II, IIIa, IIIb, IIIc, IV, V, VI, VII, VIII, IX, Xa, Xb, and Xc, wherein said defense system comprises an anti-phage, anti-conjugation, and/or anti-plasmid activity, the system comprising at least one component of said Defense System Ia, Ib, II, IIIa, IIIb, IIIc, IV, V, VI, VII, VIII, IX, Xa, Xb, or Xc, said at least one component selected from the group consisting of the components of the Defense Systems Ia, Ib, II, IIIa, IIIb, IIIc, IV, V, VI, VII, VIII, IX, Xa, Xb, or Xc, respectively, thereby protecting the bacteria from phage infection, conjugative elements, and/or plasmid transformation. In some embodiments, a method of protecting bacteria from phage infection, conjugative elements, and plasmid transformation comprises introducing into the bacteria a defense system comprising a combination of Defense Systems selected from Defense System Ia, Ib, II, IIIa, IIIb, IIIc, IV, V, VI, VII, VIII, IX, Xa, Xb, and Xc, wherein said defense system comprises an anti-phage, anti-conjugation, and anti-plasmid activity, the system comprising at least one component of each of said Defense Systems in combination, said at least one component selected from the group consisting of the components of the Defense Systems Ia, Ib, II, IIIa, IIIb, IIIc, IV, V, VI, VII, VIII, IX, Xa, Xb, or Xc, respectively, thereby protecting the bacteria from phage infection, bacterial conjugation, and/or plasmid transformation.

In some embodiments, an at least one component comprises the components of a Defense System Ia, Ib, II, IIIa, IIIb, IIIc, IV, V, VI, VII, VIII, IX, Xa, Xb, or Xc, required for a functional Defense System, wherein said function comprises an anti-phage, anti-conjugation, and/or anti-plasmid activity. In some embodiments, an at least one component comprises the components of the cassette of genes making up a Defense System Ia, Ib, II, IIIa, IIIb, IIIc, IV, V, VI, VII, VIII, IX, Xa, Xb, or Xc. In some embodiments, an at least one component comprises two components of a Defense System Ia, Ib, II, IIIa, IIIb, IIIc, IV, V, VI, VII, VIII, IX, Xa, Xb, or Xc. In some embodiments, an at least one component comprises three components of a Defense System Ia, Ib, II, IIIa, IIIb, IIIc, IV, V, VI, VII, VIII, IX, Xa, Xb, or Xc. In some embodiments, an at least one component comprises four components of a Defense System Ia, Ib, II, IIIa, IIIb, IIIc, IV, V, VI, VII, VIII, IX, Xa, Xb, or Xc. In some embodiments, an at least one component comprises five components of a Defense System Ia, Ib, II, IIIa, IIIb, IIIc, IV, V, VI, VII, VIII, IX, Xa, Xb, or Xc. In some embodiments, a component comprises the full-length component. In some embodiments, a component comprises a functional portion of the component. In some embodiments, a component comprises a component homolog.

The components of Defense Systems Ia, Ib, II, IIIa, IIIb, IIIc, IV, V, VI, VII, VIII, IX, Xa, Xb, and Xc have been described in detail above, as has the combination of these Defense systems. Methods of protecting bacteria comprising introducing into the bacteria a defense system comprising at least one components of a Defense System selected from Defense Systems Ia, Ib, II, IIIa, IIIb, IIIc, IV, V, VI, VII, VIII, IX, Xa, Xb, and Xc, or a combination of Defense Systems thereof, comprises use of the components of the Defense Systems as described in detail above, and in Tables 6-18. The detailed description of the Defense Systems and components thereof, and combinations thereof, as described above, are incorporated in full in the method of use of protecting bacteria described herein. Briefly, a non-limiting description of Defense Systems and their components includes Defense Systems Ia, Ib, II, IIIa, IIIb, IIIc, IV, V, VI, VII, VIII, IX, Xa, Xb, and Xc and components thereof, including but are not limited to methods of protecting bacteria comprising introducing into the bacteria a defense system comprising at least one components of a Defense System selected from Defense Systems Ia, Ib, II, IIIa, IIIb, IIIc, IV, V, VI, VII, VIII, IX, Xa, Xb, and Xc, or a combination of Defense Systems thereof, wherein in some embodiments:

a Defense System Ia comprises a nucleic acid construct comprising a nucleic acid sequence encoding a ZorA polypeptide comprising a pfam01618 domain, a ZorB polypeptide comprising a pfam13677 domain or a pfam00691 domain or a combination thereof, a ZorC polypeptide comprising a pfam15611 domain, and a ZorD polypeptide comprising a pfam00176 domain or a pfam00271 domain or a combination thereof;

a Defense System Ib comprises a nucleic acid construct comprising a nucleic acid sequence encoding a ZorA polypeptide comprising a pfam01618 domain, a ZorB polypeptide comprising a pfam13677 domain or a pfam00691 domain or a combination thereof, and a ZorE polypeptide comprising a pfam01844 domain or a COG3183 domain or a combination thereof;

a Defense System II comprises a nucleic acid construct comprising a nucleic acid sequence encoding a ThsA polypeptide comprising a pfam13289 domain or a pfam14519 domain or a combination thereof, and a KwaB polypeptide comprising a pfam13676 domain or a pfam08937 domain or a pfam08357 domain, or a combination thereof;

a Defense System IIIa comprises a nucleic acid construct comprising a nucleic acid sequence encoding a DruA polypeptide comprising a DUF4338 domain or a pfam14236 domain or a combination thereof, a DruB polypeptide, a DruC polypeptide, a DruD polypeptide, and a DruE polypeptide comprising a pfam00270 domain or a pfam00271 domain or a pfam09369 domain or a DUF1998 domain or any combination thereof;

a Defense System IIIa comprises a nucleic acid construct comprising a nucleic acid sequence encoding a DruA polypeptide comprising a DUF4338 domain or a pfam14236 domain or a combination thereof, a DruB polypeptide, a DruC polypeptide, a DruD polypeptide, and a DruE polypeptide comprising a pfam00270 domain or a pfam00271 domain or a pfam09369 domain or a DUF1998 domain or any combination thereof, said DruB polypeptide encoded by a gene positioned within 5 genes of a gene encoding DruA, DruD, and or DruE, and said DruC polypeptide encoded by a gene positioned within 5 genes of a gene encoding DruA, DruD, and or DruE;

a Defense System IIIb comprises a nucleic acid construct comprising a nucleic acid sequence encoding a DruM polypeptide comprising a pfam00145 domain or a COG0270 or a combination thereof, a DruF polypeptide, a DruG polypeptide, and a DruE polypeptide comprising a pfam00270 domain or a pfam00271 domain or a pfam09369 domain or a DUF1998 domain or any combination thereof;

a Defense System IIIb comprises a nucleic acid construct comprising a nucleic acid sequence encoding a DruM polypeptide comprising a pfam00145 domain or a COG0270 or a combination thereof, a DruF polypeptide, a DruG polypeptide, and a DruE polypeptide comprising a pfam00270 domain or a pfam00271 domain or a pfam09369 domain or a DUF1998 domain or any combination thereof, said DruF polypeptide encoded by a gene positioned within 5 genes of a gene encoding DruM and or DruE, and said DruG polypeptide encoded by a gene positioned within 5 genes of a gene encoding DruM and or DruE;

a Defense System IIIc comprises a nucleic acid construct comprising a nucleic acid sequence encoding a DruH polypeptide and a DruE polypeptide comprising a pfam00270 domain or a pfam00271 domain or a pfam09369 domain or a DUF1998 domain or any combination thereof;

a Defense System IIIc comprises a nucleic acid construct comprising a nucleic acid sequence encoding a DruH polypeptide and a DruE polypeptide comprising a pfam00270 domain or a pfam00271 domain or a pfam09369 domain or a DUF1998 domain or any combination thereof, said DruH polypeptide encoded by a gene positioned within 5 genes of a gene encoding DruE;

a Defense System IV comprises a nucleic acid construct comprising a nucleic acid sequence encoding a HamA polypeptide comprising a pfam08878 domain or a DUF1837 domain or a combination thereof, and a HamB polypeptide comprising COG1204 domain or a pfam00270 domain or a pfam00271 domain or any combination thereof;

a Defense System V comprises a nucleic acid construct comprising a nucleic acid sequence encoding a SduA polypeptide comprising a pfam14082 domain or a pfam01939 domain or a combination thereof;

a Defense System VI comprises a nucleic acid construct comprising a nucleic acid sequence encoding a GajA polypeptide comprising a pfam13175 domain or a COG3593 domain or a combination thereof, and a GajB polypeptide comprising a pfam04257 domain or a pfam00580 domain or a pfam13361 domain or a combination thereof;

a Defense System VII comprises a nucleic acid construct comprising a nucleic acid sequence encoding a PtuA polypeptide comprising a pfam13304 domain or a pfam02463 domain or a combination thereof, and a PtuB polypeptide comprising pfam13395 domain or a pfam 01844 domain or a combination thereof;

a Defense System VIII comprises a nucleic acid construct comprising a nucleic acid sequence encoding a LmuA polypeptide comprising a pfam14130 domain or a DUF4297 domain or a combination thereof, and a LmuB polypeptide comprising a pfam02463 domain;

a Defense System IX comprises a nucleic acid construct comprising a nucleic acid sequence encoding a KwaA polypeptide and a KwaB polypeptide comprising a pfam16162 domain or a DUF4868 domain or a combination thereof;

a Defense System IX comprises a nucleic acid construct comprising a nucleic acid sequence encoding a KwaA polypeptide and a KwaB polypeptide comprising a pfam16162 domain or a DUF4868 domain or a combination thereof, said KwaA polypeptide encoded by a gene positioned within 5 genes of a gene encoding KwaB;

a Defense System Xa comprises a nucleic acid construct comprising a nucleic acid sequence encoding a JetA polypeptide comprising a pfam11855 domain or a DUF3375 domain or any combination thereof, a JetB polypeptide comprising a pfam13835 domain or a DUF4194 domain or any combination thereof, a JetC polypeptide comprising a pfam13555 domain or a pfam13558 domain or a COG4913 domain or a combination thereof, and a JetD polypeptide comprising a pfam11795 domain or a DUF3322 domain or a pfam09983 domain or a DUF2220 domain or a COG4924 domain or any combination thereof;

a Defense System Xb comprises a nucleic acid construct comprising a nucleic acid sequence encoding a JetAⁱⁱ polypeptide comprising a pfam09660 domain or a DUF2397 domain or any combination thereof, a JetBⁱⁱ polypeptide comprising a pfam09661 domain or a DUF2398 domain or any combination thereof, a JetCⁱⁱ polypeptide comprising a pfam13558 domain, and a JetDⁱⁱ polypeptide comprising a pfam11796 domain or a DUF3323 domain or a pfam09664 domain or a DUF2399 domain or any combination thereof;

a Defense System Xc comprises a nucleic acid construct comprising a nucleic acid sequence encoding a JetAⁱⁱⁱ polypeptide, a JetBⁱⁱⁱ polypeptide, a JetCⁱⁱⁱ polypeptide comprising a COG1196 domain, a JetDⁱⁱⁱ polypeptide comprising a pfam09983 domain or a pfam09664 domain, or a DUF2220 domain, or a DUF2399 domain, or a combination thereof; or

a Defense System Xc comprises a nucleic acid construct comprising a nucleic acid sequence encoding a JetAⁱⁱⁱ polypeptide, a JetBⁱⁱⁱ polypeptide, a JetCⁱⁱⁱ polypeptide comprising a COG1196 domain, a JetDⁱⁱⁱ polypeptide comprising a pfam09983 domain or a pfam09664 domain, or a DUF2220 domain, or a DUF2399 domain, or a combination thereof, said JetAⁱⁱⁱ polypeptide and a JetBⁱⁱⁱ polypeptide each encoded by a gene positioned within 5 genes of a gene encoding JetCⁱⁱⁱ polypeptide or a JetDⁱⁱⁱ polypeptide;

wherein each nucleic acid construct of each Defense System Ia-Xc further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof; wherein said method results in protecting bacteria comprising the bacteria being resistant to at least one phage, or the bacteria having reduced efficacy of plasmid transformation, or a combination thereof.

In some embodiments, the bacteria do not express an endogenous version of the defense system being introduced. In some embodiments, the bacteria do not express a functional endogenous version of the defense system being introduced.

In some embodiments, the bacteria do not express an endogenous version of the Defense System Ia. In some embodiments, the bacteria do not express a functional endogenous version of the Defense System Ia. In some embodiments, the bacteria do not express an endogenous version of the Defense System Ib. In some embodiments, the bacteria do not express a functional endogenous version of the Defense System Ib. In some embodiments, the bacteria do not express an endogenous version of the Defense System II. In some embodiments, the bacteria do not express a functional endogenous version of the Defense System II. In some embodiments, the bacteria do not express an endogenous version of the Defense System IIIa. In some embodiments, the bacteria do not express a functional endogenous version of the Defense System IIIa. In some embodiments, the bacteria do not express an endogenous version of the Defense System IIIb. In some embodiments, the bacteria do not express a functional endogenous version of the Defense System IIIb. In some embodiments, the bacteria do not express an endogenous version of the Defense System IIIc. In some embodiments, the bacteria do not express a functional endogenous version of the Defense System IIIc. In some embodiments, the bacteria do not express an endogenous version of the Defense System IV. In some embodiments, the bacteria do not express a functional endogenous version of the Defense System IV. In some embodiments, the bacteria do not express an endogenous version of the Defense System V. In some embodiments, the bacteria do not express a functional endogenous version of the Defense System V. In some embodiments, the bacteria do not express an endogenous version of the Defense System VI. In some embodiments, the bacteria do not express a functional endogenous version of the Defense System VI. In some embodiments, the bacteria do not express an endogenous version of the Defense System VII. In some embodiments, the bacteria do not express a functional endogenous version of the Defense System VII. In some embodiments, the bacteria do not express an endogenous version of the Defense System VIII. In some embodiments, the bacteria do not express a functional endogenous version of the Defense System VIII. In some embodiments, the bacteria do not express an endogenous version of the Defense System IX. In some embodiments, the bacteria do not express a functional endogenous version of the Defense System IX. In some embodiments, the bacteria do not express an endogenous version of the Defense System Xa. In some embodiments, the bacteria do not express a functional endogenous version of the Defense System Xa. In some embodiments, the bacteria do not express an endogenous version of the Defense System Xb. In some embodiments, the bacteria do not express a functional endogenous version of the Defense System Xb. In some embodiments, the bacteria do not express an endogenous version of the Defense System Xc. In some embodiments, the bacteria do not express a functional endogenous version of the Defense System Xc.

In some embodiments, the bacteria do not express an endogenous version of the combination of Defense Systems selected from combinations disclosed herein above, of any of Defense Systems Ia-Xc. In some embodiments, the bacteria do not express a functional endogenous version of the combination of Defense Systems selected from combinations disclosed herein above, of any of Defense Systems Ia-Xc.

A skilled artisan would appreciate that various modalities may be used to introduce or express a defense system comprising a Defense System Ia-Xc or a combination thereof, or the components of a Defense System Ia-Xc, in the bacteria.

In some embodiments, the method is effected by expressing in the bacteria, an isolated nucleic acid sequence(s), nucleic acid construct or construct or alternatively introducing the component polypeptides of a Defense System or a combination of Defense Systems, as described herein, to confer protection. In some embodiments, protection comprises an anti-phage activity. In some embodiments, protection comprises an anti-plasmid activity. In some embodiments, protection comprises a combination of an anti-phage activity and an anti-plasmid activity. Alternatively, or additionally, the defense system comprising a Defense System or a combination of Defense Systems, is introduced into the bacteria via transmissible genetic element in a process of bacterial conjugation.

In some embodiments, there is provided a method of protecting bacteria from phage infection, or plasmid transformation, or phage infection and plasmid transformation, the method comprising introducing into the bacteria a defense system having an anti-phage activity, or an anti-plasmid activity, or an anti-phage activity and an anti-plasmid activity, wherein said bacteria is a first bacteria and said introducing into said bacteria said defense system comprising a Defense System selected from Defense Systems Ia-Xc or a combination of Defense Systems selected from Defense Systems Ia-Xc, comprises contacting said first bacteria with a second bacteria expressing said defense system comprising a Defense System selected from Defense Systems Ia-Xc or a combination of Defense Systems selected from Defense Systems Ia-Xc on a transmissible genetic element.

In some embodiments, a transmissible genetic element comprises a defense system comprising a Defense System selected from Defense Systems Ia-Xc or a combination of Defense Systems selected from Defense Systems Ia-Xc, as described herein in detail above. In some embodiments, the transmissible genetic element comprises a conjugative genetic element or a mobilized genetic element. In some embodiments, a transmissible genetic element comprises a plasmid or a transposon.

A skilled artisan would appreciate that the phrase “bacterial conjugation” encompasses a direct transfer of genetic material between bacterial cells by cell-to-cell contact or by bridge-like connection between the cells. During conjugation the donor bacterium provides a transmissible genetic element, typically a plasmid or a transposon. The transfer of the transmissible genetic element takes advantage of the complementary nature of double stranded DNA. Thus, one strand of the transmissible genetic element is transferred, and the other remains in the original bacteria. Both strands have the complementary stranded added so that each bacterium ends up with a complete transmissible element.

According to some embodiments, contacting a first bacteria with second bacteria comprise the step of incubating the bacterial cell (e.g., first bacteria) with a substance or cell (e.g., second bacteria) such that the substance or a substance contained in the cell affects the bacterial cell resistance to phage infection, or to plasmid transformation, or to phage infection and plasmid transformation.

In some embodiments, the first bacteria and the second bacteria are non-identical.

In some embodiments, the first bacteria do not express an endogenous defense system comprising a Defense System selected from Defense Systems Ia-Xc or a combination of Defense Systems selected from Defense Systems Ia-Xc. In some embodiments, the first bacteria do not express an endogenous functional defense system comprising a Defense System selected from Defense Systems Ia-Xc or a combination of Defense Systems selected from Defense Systems Ia-Xc.

In some embodiments, the first bacteria is a commercially valuable bacteria such as those used for fermentation as described above. Thus, following the above teachings, in some embodiments, there is provided isolated bacteria comprising a nucleic acid sequence encoding a defense system comprising a Defense System selected from Defense Systems Ia-Xc or a combination of Defense Systems selected from Defense Systems Ia-Xc, said system having an anti-phage activity and a transmissible genetic element expressing the defense system comprising a Defense System selected from Defense Systems Ia-Xc or a combination of Defense Systems selected from Defense Systems Ia-Xc, wherein the isolated bacteria does not express an endogenous functional defense system comprising a Defense System selected from Defense Systems Ia-Xc or a combination of Defense Systems selected from Defense Systems Ia-Xc.

In some embodiments, the first bacteria is a commercially valuable bacteria such as those used for fermentation as described above. Thus, following the above teachings, in some embodiments, there is provided isolated bacteria comprising a nucleic acid sequence encoding a defense system comprising a Defense System selected from Defense Systems Ia-Xc or a combination of Defense Systems selected from Defense Systems Ia-Xc, said system having an anti-plasmid activity and a transmissible genetic element expressing the defense system comprising a Defense System selected from Defense Systems Ia-Xc or a combination of Defense Systems selected from Defense Systems Ia-Xc, wherein the isolated bacteria does not express an endogenous functional defense system comprising a Defense System selected from Defense Systems Ia-Xc or a combination of Defense Systems selected from Defense Systems Ia-Xc.

In some embodiments, the first bacteria is a commercially valuable bacteria such as those used for fermentation as described above. Thus, following the above teachings, in some embodiments, there is provided isolated bacteria comprising a nucleic acid sequence encoding a defense system comprising a Defense System selected from Defense Systems Ia-Xc or a combination of Defense Systems selected from Defense Systems Ia-Xc, said system having an anti-phage activity and anti-plasmid activity, and a transmissible genetic element expressing the defense system comprising a Defense System selected from Defense Systems Ia-Xc or a combination of Defense Systems selected from Defense Systems Ia-Xc, wherein the isolated bacteria does not express an endogenous functional defense system comprising a Defense System selected from Defense Systems Ia-Xc or a combination of Defense Systems selected from Defense Systems Ia-Xc.

In some embodiments, a cell comprises a host cell. In some embodiments, a host cell comprises bacteria. In some embodiments, a cell comprises bacteria. In some embodiments, a host cell comprises a microbial cell. In some embodiments, a cell comprises a microbial cell. In some embodiments, the cell is an isolated cell. In some embodiments, bacteria are isolated bacteria. In some embodiments, a cell comprises isolated microbial cells. In some embodiments, the cell is a microbial cell such as bacteria, e.g., Gram-positive or Gram-negative bacteria. In some embodiments, the bacteria comprise Gram-negative bacteria or Negativicutes that stain negative in Gram stain. In some embodiments, a host cell comprises gram-positive bacteria, gram-negative bacteria, or archaea.

In some embodiments, Gram-negative bacteria comprise Acinetobacter calcoaceticus, Actinobacillus actinomycetemcomitans, Aeromonas hydrophila, Alcaligenes xylosoxidans, Bacteroides, Bacteroides fragilis, Bartonella bacilliformis, Bordetella spp., Borrelia burgdorferi, Branhamella catarrhalis, Brucella spp., Campylobacter spp., Chalmydia pneumoniae, Chlamydia psittaci, Chlamydia trachomatis, Chromobacterium violaceum, Citrobacter spp., Eikenella corrodens, Enterobacter aerogenes, Escherichia coli, Flavobacterium meningosepticum, Fusobacterium spp., Haemophilus influenzae, Haemophilus spp., Helicobacter pylori, Klebsiella spp., Legionella spp., Leptospira spp., Moraxella catarrhalis, Morganella morganii, Mycoplasma pneumoniae, Neisseria gonorrhoeae, Neisseria meningitidis, Pasteurella multocida, Plesiomonas shigelloides, Prevotella spp., Proteus spp., Providencia rettgeri, Pseudomonas aeruginosa, Pseudomonas spp., Rickettsia prowazekii, Rickettsia rickettsii, Rochalimaea spp., Salmonella spp., Salmonella typhi, Serratia marcescens, Shigella spp., Treponema carateum, Treponema pallidum, Treponema pallidum endemicum, Treponema pertenue, Veillonella spp., Vibrio cholerae, Vibrio vulnificus, Yersinia enterocolitica, Yersinia pestis.

In some embodiments, the bacteria comprise gammaproteobacteria (e.g. Escherichia coli, pseudomonas, vibrio and klebsiella) or Firmicutes (belonging to class Negativicutes that stain negative in Gram stain).

In some embodiments, Gram-positive bacteria comprise Actinomyces spp., Bacillus anthracis, Bifidobacterium spp., Clostridium botulinum, Clostridium perfringens, Clostridium spp., Clostridium tetani, Corynebacterium diphtheriae, Corynebacterium jeikeium, Enterococcus faecalis, Enterococcus faecium, Erysipelothrix rhusiopathiae, Eubacterium spp., Gardnerella vaginalis, Gemella morbillorum, Leuconostoc spp., Mycobacterium abcessus, Mycobacterium avium complex, Mycobacterium chelonae, Mycobacterium fortuitum, Mycobacterium haemophilium, Mycobacterium kansasii, Mycobacterium leprae, Mycobacterium marinum, Mycobacterium scrofulaceum, Mycobacterium smegmatis, Mycobacterium terrae, Mycobacterium tuberculosis, Mycobacterium ulcerans, Nocardia spp., Peptococcus niger, Peptostreptococcus spp., Proprionibacterium spp., Staphylococcus aureus, Staphylococcus auricularis, Staphylococcus capitis, Staphylococcus cohnii, Staphylococcus epidermidis, Staphylococcus haemolyticus, Staphylococcus hominis, Staphylococcus lugdanensis, Staphylococcus saccharolyticus, Staphylococcus saprophyticus, Staphylococcus schleiferi, Staphylococcus similans, Staphylococcus warneri, Staphylococcus xylosus, Streptococcus agalactiae (group B streptococcus), Streptococcus anginosus, Streptococcus bovis, Streptococcus canis, Streptococcus equi, Streptococcus milleri, Streptococcus mitior, Streptococcus mutans, Streptococcus pneumoniae, Streptococcus pyogenes (group A streptococcus), Streptococcus salivarius, Streptococcus sanguis.

In some embodiments the bacteria is a species selected from the group consisting of Escherichia, Shigella, Salmonella, Erwinia, Yersinia, Bacillus, Vibrio, Legionella, Pseudomonas, Neisseria, Bordetella, Helicobacter, Listeria, Agrobacterium, Staphylococcus, Streptococcus, Enterococcus, Clostridium, Corynebacterium, Mycobacterium, Treponema, Borrelia, Francisella, Brucella, Campylobacter, Klebsiella, Frankia, Bartonella, Rickettsia, Shewanella, Serratia, Enterobacter, Proteus, Providencia, Brochothrix, and Brevibacterium.

In some embodiments, there is provided herein a cell (e.g., bacteria) expressing at least one component of a defense system. In some embodiments, the cell is genetically modified. In some embodiments, the cell comprises an isolated cell. The components of defense systems comprising a Defense System Ia-Xc and combinations thereof, have been described in detail above. In some embodiments, polypeptide components of these systems include but are not limited to ZorA, ZorB, ZorC, ZorD, ZorE (Defense System Ia and Ib); ThsA and ThsB (Defense System II); DruA, DruB, DruC, DruD, DruE, DruH, DruM, DruG, DruF (Defense Systems IIIa, IIIb, IIIc); HamA and HamB (Defense System IV); SduA (Defense System V); GajA and GajB (Defense System VI); PtuA and PtuB (Defense System VII); LmuA and LmuB (Defense System VIII); KwaA and KwaB (Defense System IX); and JetA, JetB, JetC, and JetD, and variants thereof (Defense Systems Xa, Xb, Xc). In some embodiments, polynucleotide (e.g., gene) components, include but are not limited to zorA, zorB, zorC, zorD, zorE (Defense Systems Ia, Ib); thsA and thsB (Defense System II); druA, druB, druC, druD, druE, druF, druG, druH, druM (Defense Systems IIIa, IIIb, IIIc); hamA and hamB (Defense System IV); sduA (Defense System V); gajA and gajB (Defense System VI); ptuA and ptuB (Defense System VII); lmuA and lmuB (Defense System VIII); kwaA and kwaB (Defense System IX); and jetA, jetB, jetC, and jetD and variants thereof (Defense Systems Xa, Xb, Xc).

In some embodiments, a cell is genetically modified to express at least 2, at least 3, or at least 4 of the components of an anti-nucleic acid defense system comprising a Defense System of Ia-Xc, or a combination thereof. In some embodiments, the cell is genetically modified to express a functional anti-nucleic acid defense system. In some embodiments, the cell is genetically modified to express the components necessary for a functional anti-nucleic acid defense system. In some embodiments, the cell is genetically modified to expression any combination of components of a Defense System Ia, any combination of components of a Defense System Ib, any combination of components of a Defense System II, any combination of components of a Defense System IIIa, any combination of components of a Defense System IIIb, any combination of components of a Defense System IIIc, any combination of components of a Defense System IV, any combination of components of a Defense System V, any combination of components of a Defense System VI, any combination of components of a Defense System VII, any combination of components of a Defense System VIII, any combination of components of a Defense System IX, any combination of components of a Defense System Xa, any combination of components of a Defense System Xb, or any combination of components of a Defense System Xc. In some embodiments, a cell is genetically modified to express all of the components of a Defense System.

In some embodiments, a cell is genetically modified to express at least 2, 3, 4, or 5 components of a Defense System or combination of Defense systems, as described herein. In some embodiments, a cell is genetically modified to express at least 2, 3, 4, or 5 functional components of a Defense System or combination of Defense systems, as described herein, wherein the cell comprises the anti-phage, or anti-plasmid, or anti-phage and anti-plasmid resistance provide by each of the functional Defense Systems.

In some embodiments, a cell that has been genetically modified to express a defense system comprising a Defense System of Ia-Xc or a combination thereof, has an anti-phage activity. In some embodiments, a cell that has been genetically modified to express a defense system comprising a Defense System of Ia-Xc or a combination thereof, has an anti-plasmid activity. In some embodiments, a cell that has been genetically modified to express a defense system comprising a Defense System of Ia-Xc or a combination thereof, has an anti-phage activity and an anti-plasmid activity.

In some embodiments, a cell (e.g., bacterial cell) does not express an endogenous defense system comprising any one of Defense Systems Ia-Xc, or any combination thereof. In some embodiments, the cell expressing a defense system described herein (e.g., bacterial cell expressing a Defense System of Ia-Xc, or any combination thereof) does not endogenously express the defense system comprising any one of a the Defense Systems of Ia-Xc, or any combination thereof.

In some embodiments, the cell expressing a defense system (e.g., bacterial cell) is resistant to infection by at least one phage. In some embodiments the cell is resistant to at least one lytic phage. In some embodiments, the cell is resistant to at least one temperate (also referred as lysogenic) phage. In some embodiments, the cell is resistant to phage lysogeny. In some embodiments, the cell is resistant to phage DNA replication. In some embodiments, the cell is resistant to plasmid transformation. In some embodiments, the cell is resistant to infection by at least one phage and is resistant to plasmid transformation.

Phage contamination is now considered to be a main cause of slow fermentation or complete starter failure. The lack of bacteria which survive adequately can result in milk products which do not have a desirable taste. Thus, a goal of scientists working to produce better milk products lies in providing bacteria which have all the characteristics associated with production of a good flavor and which are able to resist infection by phages.

In some embodiments, the bacteria may be useful in the manufacture of dairy and fermentation processing such as, but not limited to, milk-derived products, such as cheeses, yogurt, fermented milk products, sour milks, and buttermilk. In some embodiments, the bacteria are useful as a part of the starter culture in the manufacture of dairy and fermentation processing. In some embodiments, the starter culture is a food grade starter culture.

A bacterium traditionally used in the production of milk products is S. thermophilus. It is particularly employed in the production of yogurt, mozzarella and Swiss type cheeses. One problem with S. thermophilus is that it is extremely sensitive to phage infection.

In some embodiments, the bacteria are lactic acid bacteria. A skilled artisan would appreciate that the term “lactic acid bacteria” encompasses Gram positive, microaerophillic or anaerobic bacteria which ferment sugar with the production of acids including lactic acid as the predominantly produced acid, acetic acid, formic acid and propionic acid.

In some embodiments, bacteria used in methods disclosed herein, are selected from a species selected from the group of the industrially most useful lactic acid bacteria comprising Lactococcus species, Streptococcus species, Lactobacillus species, Leuconostoc species, Oenococcus species, Pediococcus species, Bifidobacterium species, and Propionibacterium species. In some embodiments, bacteria protected in a method of protecting bacteria from phage infection comprises bacteria selected from a Lactococcus species, a Streptococcus species, a Lactobacillus species, a Leuconostoc species, a Oenococcus species, a Pediococcus species, a Bifidobacterium, and a Propionibacterium species. In some embodiments a method of protecting bacteria from phage infection comprises protecting a Lactococcus species of bacteria. In some embodiments a method of protecting bacteria from phage infection comprises protecting a Streptococcus species of bacteria. In some embodiments a method of protecting bacteria from phage infection comprises protecting a Lactobacillus species of bacteria. In some embodiments a method of protecting bacteria from phage infection comprises protecting a Leuconostoc species of bacteria. In some embodiments a method of protecting bacteria from phage infection comprises protecting a Oenococcus species of bacteria. In some embodiments a method of protecting bacteria from phage infection comprises protecting a Pediococcus species of bacteria. In some embodiments a method of protecting bacteria from phage infection comprises protecting a Bifidobacterium of bacteria. In some embodiments a method of protecting bacteria from phage infection comprises protecting a Propionibacterium species of bacteria.

In some embodiments, bacteria protected in a method of protecting bacteria from plasmid transformation comprises bacteria selected from a Lactococcus species, a Streptococcus species, a Lactobacillus species, a Leuconostoc species, a Oenococcus species, a Pediococcus species, a Bifidobacterium species, and a Propionibacterium species. In some embodiments, bacteria protected in a method of protecting bacteria from plasmid transformation comprises bacteria selected from a Lactococcus species, a Streptococcus species, a Lactobacillus species, a Leuconostoc species, a Oenococcus species, a Pediococcus species, a Bifidobacterium, and a Propionibacterium species. In some embodiments a method of protecting bacteria from plasmid transformation comprises protecting a Lactococcus species of bacteria. In some embodiments a method of protecting bacteria from plasmid transformation comprises protecting a Streptococcus species of bacteria. In some embodiments a method of protecting bacteria from plasmid transformation comprises protecting a Lactobacillus species of bacteria. In some embodiments a method of protecting bacteria from plasmid transformation comprises protecting a Leuconostoc species of bacteria. In some embodiments a method of protecting bacteria from plasmid transformation comprises protecting a Oenococcus species of bacteria. In some embodiments a method of protecting bacteria from plasmid transformation comprises protecting a Pediococcus species of bacteria. In some embodiments a method of protecting bacteria from plasmid transformation comprises protecting a Bifidobacterium of bacteria. In some embodiments a method of protecting bacteria from plasmid transformation comprises protecting a Propionibacterium species of bacteria.

In some embodiments, bacteria protected in a method of protecting bacteria from phage infection and plasmid transformation comprises bacteria selected from a Lactococcus species, a Streptococcus species, a Lactobacillus species, a Leuconostoc species, a Oenococcus species, a Pediococcus species, a Bifidobacterium species, and a Propionibacterium species. In some embodiments, bacteria protected in a method of protecting bacteria from phage infection and plasmid transformation comprises bacteria selected from a Lactococcus species, a Streptococcus species, a Lactobacillus species, a Leuconostoc species, a Oenococcus species, a Pediococcus species, a Bifidobacterium, and a Propionibacterium species. In some embodiments a method of protecting bacteria from phage infection and plasmid transformation comprises protecting a Lactococcus species of bacteria. In some embodiments a method of protecting bacteria from phage infection and plasmid transformation comprises protecting a Streptococcus species of bacteria. In some embodiments a method of protecting bacteria from phage infection and plasmid transformation comprises protecting a Lactobacillus species of bacteria. In some embodiments a method of protecting bacteria from phage infection and plasmid transformation comprises protecting a Leuconostoc species of bacteria. In some embodiments a method of protecting bacteria from phage infection and plasmid transformation comprises protecting a Oenococcus species of bacteria. In some embodiments a method of protecting bacteria from phage infection and plasmid transformation comprises protecting a Pediococcus species of bacteria. In some embodiments a method of protecting bacteria from phage infection and plasmid transformation comprises protecting a Bifidobacterium of bacteria. In some embodiments a method of protecting bacteria from phage infection and plasmid transformation comprises protecting a Propionibacterium species of bacteria.

In some embodiments, a host cell comprises a defense system comprising a Defense System selected from Defense Systems Ia-Xc or a combination of Defense Systems selected from Defense Systems Ia-Xc. In some embodiments, a cell comprises a defense system comprising a Defense System selected from Defense Systems Ia-Xc or a combination of Defense Systems selected from Defense Systems Ia-Xc. In some embodiments, bacteria comprise a defense system comprising a Defense System selected from Defense Systems Ia-Xc or a combination of Defense Systems selected from Defense Systems Ia-Xc.

In some embodiments, a cell comprises a transmissible genetic element comprising a defense system comprising a Defense System selected from Defense Systems Ia-Xc or a combination of Defense Systems selected from Defense Systems Ia-Xc. In some embodiments, bacteria comprise a transmissible genetic element comprising a defense system comprising a Defense System selected from Defense Systems Ia-Xc or a combination of Defense Systems selected from Defense Systems Ia-Xc.

In some embodiments, an expression vector comprises a defense system comprising a Defense System selected from Defense Systems Ia-Xc or a combination of Defense Systems selected from Defense Systems Ia-Xc. In some embodiments, a cell comprises an expression vector comprising a defense system comprising a Defense System selected from Defense Systems Ia-Xc or a combination of Defense Systems selected from Defense Systems Ia-Xc. In some embodiments, bacteria comprise an expression vector comprising a defense system comprising a Defense System selected from Defense Systems Ia-Xc or a combination of Defense Systems selected from Defense Systems Ia-Xc. In some embodiments, an expression vector comprises multiple vectors each comprising a construct comprising a Defense System selected from Ia-Xc. In some embodiments, an expression vector comprises multiple vectors each encoding a polypeptide component or functional portion of a polypeptide component of a Defense System Ia-Xc construct.

In some embodiments, a cell comprising a defense system comprising a Defense System selected from Defense Systems Ia-Xc or a combination of Defense Systems selected from Defense Systems Ia-Xc, as disclosed herein, is resistant to at least one phage. In some embodiments, a cell comprising a defense system comprising a Defense System selected from Defense Systems Ia-Xc or a combination of Defense Systems selected from Defense Systems Ia-Xc, as disclosed herein, is resistant to plasmid transformation. In some embodiments, a cell comprising a defense system comprising a Defense System selected from Defense Systems Ia-Xc or a combination of Defense Systems selected from Defense Systems Ia-Xc, as disclosed herein, is resistant to at least one phage and is resistant to plasmid transformation.

In some embodiments, a cell comprising a transmissible element comprising a defense system comprising a Defense System selected from Defense Systems Ia-Xc or a combination of Defense Systems selected from Defense Systems Ia-Xc, as disclosed herein, is resistant to at least one phage. In some embodiments, a cell comprising a transmissible element comprising a defense system comprising a Defense System selected from Defense Systems Ia-Xc or a combination of Defense Systems selected from Defense Systems Ia-Xc, as disclosed herein, is resistant to plasmid transformation. In some embodiments, a cell comprising a transmissible element comprising a defense system comprising a Defense System selected from Defense Systems Ia-Xc or a combination of Defense Systems selected from Defense Systems Ia-Xc, as disclosed herein, is resistant to at least one phage and is resistant to plasmid transformation.

In some embodiments, a cell comprising an expression vector comprising a defense system comprising a Defense System selected from Defense Systems Ia-Xc or a combination of Defense Systems selected from Defense Systems Ia-Xc, as disclosed herein, is resistant to at least one phage. In some embodiments, a cell comprising an expression vector comprising a defense system comprising a Defense System selected from Defense Systems Ia-Xc or a combination of Defense Systems selected from Defense Systems Ia-Xc, as disclosed herein, is resistant to plasmid transformation. In some embodiments, a cell comprising an expression vector comprising a defense system comprising a Defense System selected from Defense Systems Ia-Xc or a combination of Defense Systems selected from Defense Systems Ia-Xc, as disclosed herein, is resistant to at least one phage and is resistant to plasmid transformation.

Cultures, and starter cultures, in particular are used extensively in the food industry in the manufacture of fermented products including milk products (e.g., yogurt, buttermilk, and cheese), meat products, bakery products, wine, and vegetable products. The preparation of cultures is labor intensive, occupying much space and equipment, and there is a considerable risk of contamination with spoilage bacteria and/or phages during the propagation steps. The failure of bacterial cultures due to phage infection and multiplication is a major problem with the industrial use of bacterial cultures. There are many different types of phages and new strains continue to emerge. Indeed, despite advances in culture development, there is a continuing need to improve cultures for use in industry.

In some embodiments, there is provided a method of preparing a food, the method comprising adding to the food at least one defense system, said defense system comprising a Defense System selected from Defense Systems Ia-Xc or a combination of Defense Systems selected from Defense Systems Ia-Xc, thereby preparing the food. In some embodiments, a food comprises an alcoholic beverage.

In some embodiments, an alcoholic beverage comprises a wine or a sake. In some embodiments, provided herein is a method of preparing an alcoholic beverage, the method comprising adding to the alcoholic beverage at least on defense system comprising a Defense System selected from Defense Systems Ia-Xc or a combination of Defense Systems selected from Defense Systems Ia-Xc, thereby preparing the alcoholic beverage. In some embodiments, provided herein is a method of preparing a wine, the method comprising adding to the wine at least on defense system comprising a Defense System selected from Defense Systems Ia-Xc or a combination of Defense Systems selected from Defense Systems Ia-Xc, thereby preparing the wine. In some embodiments, provided herein is a method of preparing a sake, the method comprising adding to the sake at least on defense system comprising a Defense System selected from Defense Systems Ia-Xc or a combination of Defense Systems selected from Defense Systems Ia-Xc, thereby preparing the sake.

In some embodiments, a food comprises a dairy product. In some embodiments, a dairy product comprises a milk product, a sour milk, a buttermilk, a milk, a cheese, a yogurt, viili, yakult, or casein. In some embodiments, a food comprises natto.

In some embodiments, provided herein is a method of preparing a dairy product, the method comprising adding to the dairy product at least on defense system comprising a Defense System selected from Defense Systems Ia-Xc or a combination of Defense Systems selected from Defense Systems Ia-Xc, thereby preparing the dairy product. In some embodiments, provided herein is a method of preparing a milk product, the method comprising adding to the milk product at least on defense system comprising a Defense System selected from Defense Systems Ia-Xc or a combination of Defense Systems selected from Defense Systems Ia-Xc, thereby preparing the milk product. In some embodiments, provided herein is a method of preparing milk, the method comprising adding to the milk at least on defense system comprising a Defense System selected from Defense Systems Ia-Xc or a combination of Defense Systems selected from Defense Systems Ia-Xc, thereby preparing the milk. In some embodiments, provided herein is a method of preparing a sour milk, the method comprising adding to the sour milk at least on defense system comprising a Defense System selected from Defense Systems Ia-Xc or a combination of Defense Systems selected from Defense Systems Ia-Xc, thereby preparing the sour milk. In some embodiments, provided herein is a method of preparing a butter milk, the method comprising adding to the butter milk at least on defense system comprising a Defense System selected from Defense Systems Ia-Xc or a combination of Defense Systems selected from Defense Systems Ia-Xc, thereby preparing the butter milk. In some embodiments, provided herein is a method of preparing a cheese, the method comprising adding to the cheese at least on defense system comprising a Defense System selected from Defense Systems Ia-Xc or a combination of Defense Systems selected from Defense Systems Ia-Xc, thereby preparing the cheese. In some embodiments, provided herein is a method of preparing a yogurt the method comprising adding to the yogurt at least on defense system comprising a Defense System selected from Defense Systems Ia-Xc or a combination of Defense Systems selected from Defense Systems Ia-Xc, thereby preparing the yogurt. In some embodiments, provided herein is a method of preparing a viili the method comprising adding to the viili at least on defense system comprising a Defense System selected from Defense Systems Ia-Xc or a combination of Defense Systems selected from Defense Systems Ia-Xc, thereby preparing the viili. In some embodiments, provided herein is a method of preparing a yakult the method comprising adding to the yakult at least on defense system comprising a Defense System selected from Defense Systems Ia-Xc or a combination of Defense Systems selected from Defense Systems Ia-Xc, thereby preparing the yakult. In some embodiments, provided herein is a method of preparing a casein, the method comprising adding to the casein at least on defense system comprising a Defense System selected from Defense Systems Ia-Xc or a combination of Defense Systems selected from Defense Systems Ia-Xc, thereby preparing the casein.

In some embodiments, provided herein is a method of preparing a natto, the method comprising adding to the natto at least on defense system comprising a Defense System selected from Defense Systems Ia-Xc or a combination of Defense Systems selected from Defense Systems Ia-Xc, thereby preparing the natto.

In some embodiments, there is provided a method of preparing a food additive, the method comprising adding to the food additive, at least one defense system components, said defense system comprising a Defense System selected from Defense Systems Ia-Xc or a combination of Defense Systems selected from Defense Systems Ia-Xc, thereby preparing the food additive.

In some embodiments, there is provided a method of preparing a feed, the method comprising adding to the feed at least one defense system components, said defense system comprising a Defense System selected from Defense Systems Ia-Xc or a combination of Defense Systems selected from Defense Systems Ia-Xc, thereby preparing the feed.

In some embodiments, there is provided a method of preparing a nutritional supplement, the method comprising adding to the nutritional supplement at least one defense system components, said defense system comprising a Defense System selected from Defense Systems Ia-Xc or a combination of Defense Systems selected from Defense Systems Ia-Xc, thereby preparing the nutritional supplement.

In some embodiments, there is provided a method of preparing a probiotic supplement, the method comprising adding to the probiotic supplement, said defense system comprising a Defense System selected from Defense Systems Ia-Xc or a combination of Defense Systems selected from Defense Systems Ia-Xc, thereby preparing the probiotic supplement.

In some embodiments, there is provided a method of preparing a health care product, the method comprising adding to the health care product at least one defense system components, said defense system comprising a Defense System selected from Defense Systems Ia-Xc or a combination of Defense Systems selected from Defense Systems Ia-Xc, thereby preparing the health care product. In some embodiments, a health care product comprises an antibiotic.

In some embodiments, there is provided a method of preparing an antibiotic, the method comprising adding to the antibiotic, said defense system comprising a Defense System selected from Defense Systems Ia-Xc or a combination of Defense Systems selected from Defense Systems Ia-Xc, thereby preparing the antibiotic. In some embodiments, an antibiotic comprises a polymyxin, a colistin, or a bacitracin.

In some embodiments, there is provided a method of preparing a polymyxin, the method comprising adding to the polymyxin, said defense system comprising a Defense System selected from Defense Systems Ia-Xc or a combination of Defense Systems selected from Defense Systems Ia-Xc, thereby preparing the polymyxin. In some embodiments, there is provided a method of preparing colistin, the method comprising adding to the colistin, said defense system comprising a Defense System selected from Defense Systems Ia-Xc or a combination of Defense Systems selected from Defense Systems Ia-Xc, thereby preparing the colistin. In some embodiments, there is provided a method of preparing a bacitracin, the method comprising adding to the bacitracin, said defense system comprising a Defense System selected from Defense Systems Ia-Xc or a combination of Defense Systems selected from Defense Systems Ia-Xc, thereby preparing the bacitracin.

In some embodiments, there is provided a method of preparing a personal care product, the method comprising adding to the personal care product at least one defense system components, said defense system comprising a Defense System selected from Defense Systems Ia-Xc or a combination of Defense Systems selected from Defense Systems Ia-Xc, thereby preparing the personal care product.

In some embodiments, there is provided a method of preparing a veterinary product, the method comprising adding to the veterinary product at least one defense system components, said defense system comprising a Defense System selected from Defense Systems Ia-Xc or a combination of Defense Systems selected from Defense Systems Ia-Xc, thereby preparing veterinary product.

In some embodiments, there is provided a method of preparing a L-glutamine, the method comprising adding to the L-glutamine at least one defense system components, said defense system comprising a Defense System selected from Defense Systems Ia-Xc or a combination of Defense Systems selected from Defense Systems Ia-Xc, thereby preparing L-glutamine.

In some embodiments, there is provided a method of preparing acetone, the method comprising adding to the acetone at least one defense system components, said defense system comprising a Defense System selected from Defense Systems Ia-Xc or a combination of Defense Systems selected from Defense Systems Ia-Xc, thereby preparing acetone.

In some embodiments, there is provided a method of preparing butanol, the method comprising adding to the butanol at least one defense system components, said defense system comprising a Defense System selected from Defense Systems Ia-Xc or a combination of Defense Systems selected from Defense Systems Ia-Xc, thereby preparing butanol.

Thus, following the above teachings, in some embodiments, there is provided a food, food additive, feed, nutritional supplement, probiotic supplement, personal care product, health care product and veterinary product comprising at least one components of a defense system, said defense system comprising a Defense System selected from Defense Systems Ia-Xc or a combination of Defense Systems selected from Defense Systems Ia-Xc, as described in details herein. In some embodiments, a food, food additive, feed, nutritional supplement, probiotic supplement, personal care product, health care product and veterinary product comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a food, food additive, feed, nutritional supplement, probiotic supplement, personal care product, health care product and veterinary product comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a starter culture comprising at least one components of a defense system, said defense system comprising a Defense System selected from Defense Systems Ia-Xc or a combination of Defense Systems selected from Defense Systems Ia-Xc, as described in detail herein. In some embodiments, a started culture comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a starter culture comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a food comprising at least one components of a defense system, said defense system comprising a Defense System selected from Defense Systems Ia-Xc or a combination of Defense Systems selected from Defense Systems Ia-Xc, as described in detail herein. In some embodiments, a food comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a food comprising a functional defense system, said defense system comprising a functional Defense System selected from Defense Systems Ia-Xc or a combination of functional Defense Systems selected from Defense Systems Ia-Xc, as described in detail herein.

In some embodiments, a food comprises a dairy product. In some embodiments, a food comprises an alcoholic beverage. In some embodiments, a food comprises a natto.

In some embodiments, there is provided a dairy product comprising at least one components of a defense system, said defense system comprising a Defense System selected from Defense Systems Ia-Xc or a combination of Defense Systems selected from Defense Systems Ia-Xc, as described in detail herein. In some embodiments, a dairy product comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a dairy product comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a milk comprising at least one components of a defense system, said defense system comprising a Defense System selected from Defense Systems Ia-Xc or a combination of Defense Systems selected from Defense Systems Ia-Xc, as described in detail herein. In some embodiments, a milk comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a milk comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a milk product comprising at least one components of a defense system, said defense system comprising a Defense System selected from Defense Systems Ia-Xc or a combination of Defense Systems selected from Defense Systems Ia-Xc, as described in detail herein. In some embodiments, a milk product comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a milk product comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a sour milk comprising at least one components of a defense system, said defense system comprising a Defense System selected from Defense Systems Ia-Xc or a combination of Defense Systems selected from Defense Systems Ia-Xc, as described in detail herein. In some embodiments, a sour milk comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a sour milk comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a butter milk comprising at least one components of a defense system, said defense system comprising a Defense System selected from Defense Systems Ia-Xc or a combination of Defense Systems selected from Defense Systems Ia-Xc, as described in detail herein. In some embodiments, a butter milk comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a butter milk comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a cheese comprising at least one components of a defense system, said defense system comprising a Defense System selected from Defense Systems Ia-Xc or a combination of Defense Systems selected from Defense Systems Ia-Xc, as described in detail herein. In some embodiments, a cheese comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a cheese comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a yogurt comprising at least one components of a defense system, said defense system comprising a Defense System selected from Defense Systems Ia-Xc or a combination of Defense Systems selected from Defense Systems Ia-Xc, as described in detail herein. In some embodiments, a yogurt comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a yogurt comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a viili comprising at least one components of a defense system, said defense system comprising a Defense System selected from Defense Systems Ia-Xc or a combination of Defense Systems selected from Defense Systems Ia-Xc, as described in detail herein. In some embodiments, a viili comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a viili comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a yakult comprising at least one components of a defense system, said defense system comprising a Defense System selected from Defense Systems Ia-Xc or a combination of Defense Systems selected from Defense Systems Ia-Xc, as described in detail herein. In some embodiments, a yakult comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a yakult comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a casein comprising at least one components of a defense system, said defense system comprising a Defense System selected from Defense Systems Ia-Xc or a combination of Defense Systems selected from Defense Systems Ia-Xc, as described in detail herein. In some embodiments, a casein comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a casein comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided an alcoholic beverage comprising at least one components of a defense system, said defense system comprising a Defense System selected from Defense Systems Ia-Xc or a combination of Defense Systems selected from Defense Systems Ia-Xc, as described in detail herein. In some embodiments, an alcoholic beverage comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided an alcoholic beverage comprising a functional defense system, said defense system comprising a functional Defense System selected from Defense Systems Ia-Xc or a combination of functional Defense Systems selected from Defense Systems Ia-Xc, as described in detail herein. In some embodiments, an alcoholic beverage comprises a wine or a sake. In some embodiments, there is provided a wine comprising at least one components of a defense system, said defense system comprising a Defense System selected from Defense Systems Ia-Xc or a combination of Defense Systems selected from Defense Systems Ia-Xc, as described in detail herein. In some embodiments, a wine comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a wine comprising a functional defense system, said defense system comprising a functional Defense System selected from Defense Systems Ia-Xc or a combination of functional Defense Systems selected from Defense Systems Ia-Xc, as described in detail herein. In some embodiments, there is provided a sake comprising at least one components of a defense system, said defense system comprising a Defense System selected from Defense Systems Ia-Xc or a combination of Defense Systems selected from Defense Systems Ia-Xc, as described in detail herein. In some embodiments, a sake comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a sake comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a natto comprising at least one components of a defense system, said defense system comprising a Defense System selected from Defense Systems Ia-Xc or a combination of Defense Systems selected from Defense Systems Ia-Xc, as described in detail herein. In some embodiments, a natto comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a natto comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a food additive comprising at least one components of a defense system, said defense system comprising a Defense System selected from Defense Systems Ia-Xc or a combination of Defense Systems selected from Defense Systems Ia-Xc, as described in detail herein. In some embodiments, a food additive comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a food additive comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided feed comprising at least one components of a defense system, said defense system comprising a Defense System selected from Defense Systems Ia-Xc or a combination of Defense Systems selected from Defense Systems Ia-Xc, as described in detail herein. In some embodiments, feed comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided feed comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a nutritional supplement comprising at least one components of a defense system, said defense system comprising a Defense System selected from Defense Systems Ia-Xc or a combination of Defense Systems selected from Defense Systems Ia-Xc, as described in detail herein. In some embodiments, a nutritional supplement comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a nutritional supplement comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a probiotic supplement comprising at least one components of a defense system, said defense system comprising a Defense System selected from Defense Systems Ia-Xc or a combination of Defense Systems selected from Defense Systems Ia-Xc, as described in detail herein. In some embodiments, a probiotic supplement comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a probiotic supplement comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a health care product comprising at least one components of a defense system, said defense system comprising a Defense System selected from Defense Systems Ia-Xc or a combination of Defense Systems selected from Defense Systems Ia-Xc, as described in detail herein. In some embodiments, a health care product comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a health care product comprising a functional defense system, said defense system comprising a functional Defense System selected from Defense Systems Ia-Xc or a combination of functional Defense Systems selected from Defense Systems Ia-Xc, as described in detail herein.

In some embodiments, a health care product comprises an antibiotic. In some embodiments, there is provided an antibiotic comprising at least one components of a defense system, said defense system comprising a Defense System selected from Defense Systems Ia-Xc or a combination of Defense Systems selected from Defense Systems Ia-Xc, as described in detail herein. In some embodiments, an antibiotic comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided an antibiotic comprising a functional defense system, said defense system comprising a functional Defense System selected from Defense Systems Ia-Xc or a combination of functional Defense Systems selected from Defense Systems Ia-Xc, as described in detail herein.

In some embodiments, an antibiotic comprises a polymyxin, a colistin, or a bacitracin. In some embodiments, there is provided a polymyxin comprising at least one components of a defense system, said defense system comprising a Defense System selected from Defense Systems Ia-Xc or a combination of Defense Systems selected from Defense Systems Ia-Xc, as described in detail herein. In some embodiments, a polymyxin comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a polymyxin comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a colistin comprising at least one components of a defense system, said defense system comprising a Defense System selected from Defense Systems Ia-Xc or a combination of Defense Systems selected from Defense Systems Ia-Xc, as described in detail herein. In some embodiments, a colistin comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a colistin comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a bacitracin comprising at least one components of a defense system, said defense system comprising a Defense System selected from Defense Systems Ia-Xc or a combination of Defense Systems selected from Defense Systems Ia-Xc, as described in detail herein. In some embodiments, a bacitracin comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a bacitracin comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a personal care product comprising at least one components of a defense system, said defense system comprising a Defense System selected from Defense Systems Ia-Xc or a combination of Defense Systems selected from Defense Systems Ia-Xc, as described in detail herein. In some embodiments, a personal care product comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a personal care product comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a veterinary product comprising at least one components of a defense system, said defense system comprising a Defense System selected from Defense Systems Ia-Xc or a combination of Defense Systems selected from Defense Systems Ia-Xc, as described in detail herein. In some embodiments, a veterinary product comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a veterinary product comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided a L-glutamine comprising at least one components of a defense system, said defense system comprising a Defense System selected from Defense Systems Ia-Xc or a combination of Defense Systems selected from Defense Systems Ia-Xc, as described in detail herein. In some embodiments, a L-glutamine comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a L-glutamine comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided an acetone comprising at least one components of a defense system, said defense system comprising a Defense System selected from Defense Systems Ia-Xc or a combination of Defense Systems selected from Defense Systems Ia-Xc, as described in detail herein. In some embodiments, an acetone comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided an acetone comprising a functional defense system, said defense system comprising a functional Defense System selected from Defense Systems Ia-Xc or a combination of functional Defense Systems selected from Defense Systems Ia-Xc, as described in detail herein.

In some embodiments, there is provided butanol comprising at least one components of a defense system, said defense system comprising a Defense System selected from Defense Systems Ia-Xc or a combination of Defense Systems selected from Defense Systems Ia-Xc, as described in detail herein. In some embodiments, a butanol comprises at least one functional component of a defense system, said defense system comprising a Defense System selected from Defense Systems Ia-Xc or a combination of Defense Systems selected from Defense Systems Ia-Xc, as described in detail herein. In some embodiments, there is provided a butanol comprising a functional defense system, said defense system comprising a functional Defense System selected from Defense Systems Ia-Xc or a combination of functional Defense Systems selected from Defense Systems Ia-Xc, as described in detail herein.

In some embodiments, the nucleic acid construct of the Defense Systems Ia-Xb comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof.

In some embodiments, a method of preparing a food, food additive, feed, nutritional supplement, probiotic supplement, personal care product, health care product and veterinary product, comprises adding to the food, food additive, feed, nutritional supplement, probiotic supplement, personal care product, health care product and veterinary product at least one component selected from the group consisting of the components of the defense system, thereby preparing a food, food additive, feed, nutritional supplement, probiotic supplement, personal care product, health care product and veterinary product. In some embodiments, a method of preparing a food, food additive, feed, nutritional supplement, probiotic supplement, personal care product, health care product and veterinary product comprises introducing into the bacteria a defense system comprising a Defense System selected from a Defense System Ia, Ib, II, IIIa, IIIb, IIIc, IV, V, VI, VII, VIII, IX, Xa, Xb, and Xc, wherein said defense system comprises an anti-phage activity, or an anti-plasmid activity, or an anti-phage and an anti-plasmid activity, the system comprising at least one component of said Defense System Ia, Ib, II, IIIa, IIIb, IIIc, IV, V, VI, VII, VIII, IX, Xa, Xb, or Xc, said at least one component selected from the group consisting of the components of the Defense Systems Ia, Ib, II, IIIa, IIIb, IIIc, IV, V, VI, VII, VIII, IX, Xa, Xb, or Xc, respectively, thereby preparing the food, food additive, feed, nutritional supplement, probiotic supplement, personal care product, health care product and veterinary product.

In some embodiments, a method of preparing a food, food additive, feed, nutritional supplement, probiotic supplement, personal care product, health care product and veterinary product comprises introducing into the bacteria a defense system comprising a combination of Defense Systems selected from Defense System Ia, Ib, II, IIIa, IIIb, IIIc, IV, V, VI, VII, VIII, IX, Xa, Xb, and Xc, wherein said defense system comprises an anti-phage activity, an anti-plasmid activity, or an anti-phage and an anti-plasmid activity, the system comprising at least one component of each of said Defense Systems in a combination, said at least one component selected from the group consisting of the components of the Defense Systems Ia, Ib, II, IIIa, IIIb, IIIc, IV, V, VI, VII, VIII, IX, Xa, Xb, or Xc, respectively, thereby preparing a food, food additive, feed, nutritional supplement, probiotic supplement, personal care product, health care product and veterinary product.

In some embodiments, an at least one component comprises the components of a Defense System Ia, Ib, II, IIIa, IIIb, IIIc, IV, V, VI, VII, VIII, IX, Xa, Xb, or Xc, required for a functional Defense System, wherein said function comprises an anti-phage activity, an anti-plasmid activity, or an anti-phage and an anti-plasmid activity. In some embodiments, an at least one component comprises the components of the cassette of genes making up a Defense System Ia, Ib, II, IIIa, IIIb, IIIc, IV, V, VI, VII, VIII, IX, Xa, Xb, or Xc. In some embodiments, an at least one component comprises two components of a Defense System Ia, Ib, II, IIIa, IIIb, IIIc, IV, V, VI, VII, VIII, IX, Xa, Xb, or Xc. In some embodiments, an at least one component comprises three components of a Defense System Ia, Ib, II, IIIa, IIIb, IIIc, IV, V, VI, VII, VIII, IX, Xa, Xb, or Xc. In some embodiments, an at least one component comprises four components of a Defense System Ia, Ib, II, IIIa, IIIb, IIIc, IV, V, VI, VII, VIII, IX, Xa, Xb, or Xc. In some embodiments, an at least one component comprises five components of a Defense System Ia, Ib, II, IIIa, IIIb, IIIc, IV, V, VI, VII, VIII, IX, Xa, Xb, or Xc. In some embodiments, a component comprises the full-length component. In some embodiments, a component comprises a functional portion of the component. In some embodiments, a component comprises a component homolog. Defense System components, nucleic acid sequences thereof, and amino acid sequences thereof are presented herein in Table 18.

In some embodiments, for a method of use disclosed herein, the at least one defense system component is comprised in cell (e.g. bacteria) genetically modified to express the component.

In some embodiments, there is provided a method of preparing a food, food additive, feed, nutritional supplement, probiotic supplement, personal care product, health care product and veterinary product, the method comprising adding to the food, food additive, feed, nutritional supplement, probiotic supplement, personal care product, health care product and veterinary product a cell comprising a defense system disclosed herein, thereby preparing the food, food additive, feed, nutritional supplement, probiotic supplement, personal care product, health care product and veterinary product. In some embodiments, said use comprises use of an isolated cell comprising a defense system disclosed herein.

In some embodiments, there is provided a food, food additive, feed, nutritional supplement, probiotic supplement, personal care product, health care product and veterinary product comprising the cell comprising a defense system disclosed herein.

In some embodiments, there is provided a method of preparing a food, food additive, feed, nutritional supplement, probiotic supplement, personal care product, health care product and veterinary product comprising adding to the food, food additive, feed, nutritional supplement, probiotic supplement, personal care product, health care product and veterinary product bacteria which expresses on a transmissible genetic element at least one component of a defense system disclosed herein, wherein said defense system comprising a Defense System selected from Defense Systems Ia-Xc or a combination of Defense Systems selected from Defense Systems Ia-Xc, as described in detail herein, thereby preparing the food, food additive, feed, nutritional supplement, probiotic supplement, personal care product, health care product and veterinary product.

In some embodiments, an at least one component comprises the components of a Defense System Ia, Ib, II, IIIa, IIIb, IIIc, IV, V, VI, VII, VIII, IX, Xa, Xb, or Xc, required for a functional Defense System, wherein said function comprises an anti-phage activity, an anti-plasmid activity, or an anti-phage and an anti-plasmid activity. In some embodiments, an at least one component comprises the components of the cassette of genes making up a Defense System Ia, Ib, II, IIIa, IIIb, IIIc, IV, V, VI, VII, VIII, IX, Xa, Xb, or Xc. In some embodiments, an at least one component comprises two components of a Defense System Ia, Ib, II, IIIa, IIIb, IIIc, IV, V, VI, VII, VIII, IX, Xa, Xb, or Xc. In some embodiments, an at least one component comprises three components of a Defense System Ia, Ib, II, IIIa, IIIb, IIIc, IV, V, VI, VII, VIII, IX, Xa, Xb, or Xc. In some embodiments, an at least one component comprises four components of a Defense System Ia, Ib, II, IIIa, IIIb, IIIc, IV, V, VI, VII, VIII, IX, Xa, Xb, or Xc. In some embodiments, an at least one component comprises five components of a Defense System Ia, Ib, II, IIIa, IIIb, IIIc, IV, V, VI, VII, VIII, IX, Xa, Xb, or Xc. In some embodiments, a component comprises the full-length component. In some embodiments, a component comprises a functional portion of the component. In some embodiments, a component comprises a component homolog. Defense System components, nucleic acid sequences thereof, and amino acid sequences thereof are presented herein in Table 18.

In some embodiments, there is provided a food, food additive, feed, nutritional supplement, probiotic supplement, personal care product, health care product and veterinary product comprising bacteria which expresses on a transmissible genetic element at least one component of a defense system comprising a Defense System selected from Defense Systems Ia-Xc or a combination of Defense Systems selected from Defense Systems Ia-Xc.

In some embodiments, an at least one component comprises the components of a Defense System Ia, Ib, II, IIIa, IIIb, IIIc, IV, V, VI, VII, VIII, IX, Xa, Xb, or Xc, required for a functional Defense System, wherein said function comprises an anti-phage activity, an anti-plasmid activity, or an anti-phage and an anti-plasmid activity. In some embodiments, an at least one component comprises the components of the cassette of genes making up a Defense System Ia, Ib, II, IIIa, IIIb, IIIc, IV, V, VI, VII, VIII, IX, Xa, Xb, or Xc. In some embodiments, an at least one component comprises two components of a Defense System Ia, Ib, II, IIIa, IIIb, IIIc, IV, V, VI, VII, VIII, IX, Xa, Xb, or Xc. In some embodiments, an at least one component comprises three components of a Defense System Ia, Ib, II, IIIa, IIIb, IIIc, IV, V, VI, VII, VIII, IX, Xa, Xb, or Xc. In some embodiments, an at least one component comprises four components of a Defense System Ia, Ib, II, IIIa, IIIb, IIIc, IV, V, VI, VII, VIII, IX, Xa, Xb, or Xc. In some embodiments, an at least one component comprises five components of a Defense System Ia, Ib, II, IIIa, IIIb, IIIc, IV, V, VI, VII, VIII, IX, Xa, Xb, or Xc. In some embodiments, a component comprises the full-length component. In some embodiments, a component comprises a functional portion of the component. In some embodiments, a component comprises a component homolog. Defense System components, nucleic acid sequences thereof, and amino acid sequences thereof are presented herein in Table 18.

In some embodiments, a food or feed disclosed herein, comprises a dairy product. In some embodiments, a food additive comprises an additive to be added to a dairy product or to an ingredient used to prepare a dairy product.

The preparation of starter cultures of such bacteria, and methods of fermenting substrates, particularly food substrates such as milk, can be carried out in accordance with known techniques, including but not limited to those described in Mayra-Makinen and Bigret (1993) Lactic Acid Bacteria; Salminen and vonWright eds. Marcel Dekker, Inc. New York. 65-96; Sandine (1996) Dairy Starter Cultures Cogan and Accolas eds. VCH Publishers, New York. 191-206; Gilliland (1985) Bacterial Starter Cultures for Food. CRC Press, Boca Raton, Fla.

The term “fermenting” refers to the energy-yielding, metabolic breakdown of organic compounds by microorganisms that generally proceeds under anaerobic conditions and with the evolution of gas.

Products produced by fermentation which have been known to experience phage infection, and the corresponding infected fermentation bacteria, include cheddar and cottage cheese (Lactococcus lactis subsp. lactis, Lactococcus lactis subsp. cremoris), yogurt (Lactobacillus delbrueckii subsp. bulgaricus, Streptococcus thermophilus), Swiss cheese (S. thermophilus, Lactobacillus lactis, Lactobacillus helveticus), blue cheese (Leuconostoc cremoris), Italian cheese (L. bulgaricus, S. thermophilus), viili (Lactococcus lactis subsp. cremoris, Lactococcus lactis subsp. lactis biovar diacetylactis, Leuconostoc cremoris), yakult (Lactobacillus casei), casein (Lactococcus lactis subsp. cremoris), natto (Bacillus subtilis var. natto), wine (Leuconostoc oenos), sake (Leuconostoc mesenteroides), polymyxin (Bacillus polymyxa), colistin (Bacillus colistrium), bacitracin (Bacillus licheniformis), L-glutamic acid (Brevibacterium lactofermentum, Microbacterium ammoniaphilum), and acetone and butanol (Clostridium acetobutylicum, Clostridium saccharoperbutylacetonicum).

In some embodiments, provided herein are bacteria resistant to phage infection, said bacteria including but not limited to Lactococcus lactis subsp. lactis, Lactococcus lactis subsp. cremoris Lactobacillus delbrueckii subsp. bulgaricus, Streptococcus thermophilus, S. thermophilus, Lactobacillus lactis, Lactobacillus helveticus, Leuconostoc cremoris, L. bulgaricus, S. thermophilus, Lactococcus lactis subsp. cremoris, Lactococcus lactis subsp. lactis biovar diacetylactis, Leuconostoc cremoris, Lactobacillus casei, Lactococcus lactis subsp. cremoris, Bacillus subtilis var. natto, Leuconostoc oenos, Leuconostoc mesenteroides, polymyxin Bacillus polymyxa, Bacillus colistrium, Bacillus licheniformis, L-Brevibacterium lactofermentum, Microbacterium ammoniaphilum, and acetone and butanol Clostridium acetobutylicum, Clostridium saccharoperbutylacetonicum.

In some embodiments, provided here are starter cultures comprising bacteria resistant to phage infection, said bacteria including but not limited to Lactococcus lactis subsp. lactis, Lactococcus lactis subsp. cremoris Lactobacillus delbrueckii subsp. bulgaricus, Streptococcus thermophilus, S. thermophilus, Lactobacillus lactis, Lactobacillus helveticus, Leuconostoc cremoris, L. bulgaricus, S. thermophilus, Lactococcus lactis subsp. cremoris, Lactococcus lactis subsp. lactis biovar diacetylactis, Leuconostoc cremoris, Lactobacillus casei, Lactococcus lactis subsp. cremoris, Bacillus subtilis var. natto, Leuconostoc oenos, Leuconostoc mesenteroides, polymyxin Bacillus polymyxa, Bacillus colistrium, Bacillus licheniformis, L-Brevibacterium lactofermentum, Microbacterium ammoniaphilum, and acetone and butanol Clostridium acetobutylicum, Clostridium saccharoperbutylacetonicum.

In some embodiments, a method of protecting a bacteria from phage infection, comprises protecting bacteria selected from Lactococcus lactis subsp. lactis, Lactococcus lactis subsp. cremoris Lactobacillus delbrueckii subsp. bulgaricus, Streptococcus thermophilus, S. thermophilus, Lactobacillus lactis, Lactobacillus helveticus, Leuconostoc cremoris, L. bulgaricus, S. thermophilus, Lactococcus lactis subsp. cremoris, Lactococcus lactis subsp. lactis biovar diacetylactis, Leuconostoc cremoris, Lactobacillus casei, Lactococcus lactis subsp. cremoris, Bacillus subtilis var. natto, Leuconostoc oenos, Leuconostoc mesenteroides, polymyxin Bacillus polymyxa, Bacillus colistrium, Bacillus licheniformis, L-Brevibacterium lactofermentum, Microbacterium ammoniaphilum, and acetone and butanol Clostridium acetobutylicum, and Clostridium saccharoperbutylacetonicum.

In some embodiments, a method of protecting a bacteria from plasmid transformation, comprises protecting bacteria selected from Lactococcus lactis subsp. lactis, Lactococcus lactis subsp. cremoris Lactobacillus delbrueckii subsp. bulgaricus, Streptococcus thermophilus, S. thermophilus, Lactobacillus lactis, Lactobacillus helveticus, Leuconostoc cremoris, L. bulgaricus, S. thermophilus, Lactococcus lactis subsp. cremoris, Lactococcus lactis subsp. lactis biovar diacetylactis, Leuconostoc cremoris, Lactobacillus casei, Lactococcus lactis subsp. cremoris, Bacillus subtilis var. natto, Leuconostoc oenos, Leuconostoc mesenteroides, polymyxin Bacillus polymyxa, Bacillus colistrium, Bacillus licheniformis, L-Brevibacterium lactofermentum, Microbacterium ammoniaphilum, and acetone and butanol Clostridium acetobutylicum, and Clostridium saccharoperbutylacetonicum.

In some embodiments, a method of protecting a bacteria from phage infection and plasmid transformation, comprises protecting bacteria selected from Lactococcus lactis subsp. lactis, Lactococcus lactis subsp. cremoris Lactobacillus delbrueckii subsp. bulgaricus, Streptococcus thermophilus, S. thermophilus, Lactobacillus lactis, Lactobacillus helveticus, Leuconostoc cremoris, L. bulgaricus, S. thermophilus, Lactococcus lactis subsp. cremoris, Lactococcus lactis subsp. lactis biovar diacetylactis, Leuconostoc cremoris, Lactobacillus casei, Lactococcus lactis subsp. cremoris, Bacillus subtilis var. natto, Leuconostoc oenos, Leuconostoc mesenteroides, polymyxin Bacillus polymyxa, Bacillus colistrium, Bacillus licheniformis, L-Brevibacterium lactofermentum, Microbacterium ammoniaphilum, and acetone and butanol Clostridium acetobutylicum, and Clostridium saccharoperbutylacetonicum.

In some embodiments, a cell comprising a defense system described herein, comprises a Lactococcus lactis subsp. Lactis cell, a Lactococcus lactis subsp. cremoris cell, a Lactobacillus delbrueckii subsp. bulgaricus cell, a Streptococcus thermophilus cell, a S. thermophilus cell, a Lactobacillus lactis cell, a Lactobacillus helveticus cell, a Leuconostoc cremoris cell, a L. bulgaricus cell, a S. thermophilus cell, a Lactococcus lactis subsp. cremoris cell, a Lactococcus lactis subsp. lactis biovar diacetylactis cell, a Leuconostoc cremoris cell, a Lactobacillus case cell, a, Lactococcus lactis subsp. cremoris cell, a Bacillus subtilis var. natto cell, a Leuconostoc oenos cell, a Leuconostoc mesenteroides cell, a polymyxin Bacillus polymyxa cell, a Bacillus colistrium cell, a Bacillus licheniformis cell, a L-Brevibacterium lactofermentum cell, a Microbacterium ammoniaphilum cell, a Clostridium acetobutylicum cell, and a Clostridium saccharoperbutylacetonicum cell

As exemplified herein below in Examples 1-8, transformation of bacteria with a defense system confers either phage resistance or plasmid resistance to the bacteria. For example, but not limited to the results presented in FIGS. 3B, 3C, 3D, 3E, 5D, and 7C. These results suggest that the use of anti-defense system as a method to induce phage sensitivity.

In some embodiments, addition of nucleic acid sequences to the genome of a bacteria provides phage resistance. In some embodiments, addition of a nucleic acid construct, as disclosed herein, to the genome of a bacteria provides phage resistance. In some embodiments, addition of a nucleic acid construct, as disclosed herein, to the genome of a bacteria comprising Lactococcus lactis subsp. lactis, Lactococcus lactis subsp. cremoris Lactobacillus delbrueckii subsp. bulgaricus, Streptococcus thermophilus, S. thermophilus, Lactobacillus lactis, Lactobacillus helveticus, Leuconostoc cremoris, L. bulgaricus, S. thermophilus, Lactococcus lactis subsp. cremoris, Lactococcus lactis subsp. lactis biovar diacetylactis, Leuconostoc cremoris, Lactobacillus casei, Lactococcus lactis subsp. cremoris, Bacillus subtilis var. natto, Leuconostoc oenos, Leuconostoc mesenteroides, polymyxin Bacillus polymyxa, Bacillus colistrium, Bacillus licheniformis, L-Brevibacterium lactofermentum, Microbacterium ammoniaphilum, and acetone and butanol Clostridium acetobutylicum, Clostridium saccharoperbutylacetonicum, provides phage resistance to the bacteria.

As used herein, “inducing phage sensitivity” refers to an increase of at least 5% in bacterial susceptibility towards a phage, as compared to same in the absence of the defense system agent, as may be manifested e.g. in growth arrest, death or prevention of lysogeny. According to a specific embodiment, the increase is in at least 10%, 20%, 30%, 40% or even higher say, 50%, 60%, 70%, 80%, 90% or more than 100%.

### Methods of Identifying Defense Systems

Disclosed herein, in some embodiments is a computational approach to identify new defense systems localized in the vicinity of annotated defense systems.

In some embodiments, there is provided a method for identifying a defense system in a prokaryotic cell, comprising the steps of:
(a) selecting, in-silico, a gene located in close proximity to a known defense-related gene family present in a plurality of prokaryotic genomes;
(b) analyzing, in-silico, the DNA upstream and downstream of said gene; and
(c) identifying, in-silico, a cassette of genes within the upstream and downstream DNA analyzed in step (b) comprising said gene of step (a), wherein said cassette of genes has conserved synteny in said plurality of prokaryotic genomes.

In some embodiments, a method for identifying a defense system comprises the steps of:
(a) in-silico selecting a gene in location proximity to an annotated defense system of a plurality of prokaryotic genomes; and
(b) in-silico selecting a cassette of genes comprising said gene with conserved synteny in said plurality of prokaryotic genomes.

In some embodiments, the defense system comprises an anti-phage defense system. In some embodiments, the defense system comprises an anti-plasmid defense system. In some embodiments, the defense system comprises an anti-phage defense system and an anti-plasmid defense system.

In some embodiments, a gene in location proximity comprises a gene positioned within less than 10 genes upstream (5′) or 10 genes downstream (3′) to the annotated defense system. In some embodiments, the location proximity is positioned within 10 genes upstream or downstream to said annotated defense system in said prokaryotic genome.

Annotated defense systems are disclosed in scientific papers and databases known to the skilled in the art (see e.g. Makarova et al. J Bacteriol. 2011 November; 193(21): 6039-6056; Swarts et al. Nature (2014) 507, 258-261; and Goldfarb et al. EMBO J. (2015) 34, 169-83]. Non-limiting examples of annotated defense systems that can be used with some embodiments of the methods for identifying defense systems include, but are not limited to surface modifications to prevent adsorption of phages, restriction-modification (RIM) systems, infection (Abi) mechanisms, the CRISPR/Cas adaptive defense system, the prokaryotic argonaute and the BREX system.

In some embodiments, the annotated defense system is selected from the group consisting of a restriction-modification (R/M) system, an infection (Abi) system, a CRISPR/Cas adaptive defense system, a prokaryotic argonaute and a BREX system.

In some embodiments, the plurality comprises at least 2. In some embodiments, the plurality comprises at least 5, at least 10, at least 20, at least 50, at least 100 or at least 500.

A skilled artisan would appreciate that the term “conserved synteny” encompasses a conserved gene order in the genomes of different species.

In some embodiments, the method for identifying a defense system further comprises a step of validating a defensive effect of the selected (identified) cassette of genes.

Validating a defensive effect may be effected in any method known in the art.

In some embodiments, validating the defensive effect is effected by genetically modifying a prokaryotic cell to express the selected cassette of genes and subjecting the prokaryotic cell to phage infection or plasmid transformation, wherein a decrease in phage or plasmid sensitivity below a predetermined threshold as compared to a prokaryotic cell of the same origin not genetically modified to express the selected cassette of genes is indicative of a defensive effect.

In some embodiments, validating the defensive effect is effected by contacting a prokaryotic cell with an agent capable of downregulating expression and/or activity of at least one gene of the selected cassette of genes in a prokaryotic cell and subjecting the prokaryotic cell to phage infection or plasmid transformation, wherein an increase in phage or plasmid sensitivity above a predetermined threshold as compared to a prokaryotic cell of the same origin not contacted with the agent is indicative of a defensive effect.

In some embodiments, the decrease/increase below or above a predetermined threshold is statistically significant.

In some embodiments, the predetermined threshold is at least at least 1.5 fold, at least 2 fold, at least 3 fold, at least 5 fold, at least 10 fold, or at least 20 fold. In some embodiments, the predetermined threshold is at least 2%, at least 5%, at least 10%, at least 20%, at least 30%, at least 40%, at least 50%, at least 60%, at least 70%, at least 80%, at least 90%, e.g., 100%, at least 200%, at least 300%, at least 400%, at least 500%, at least 600%.

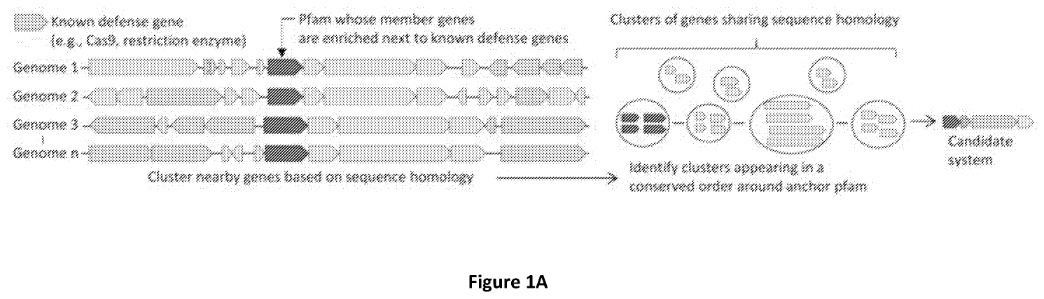

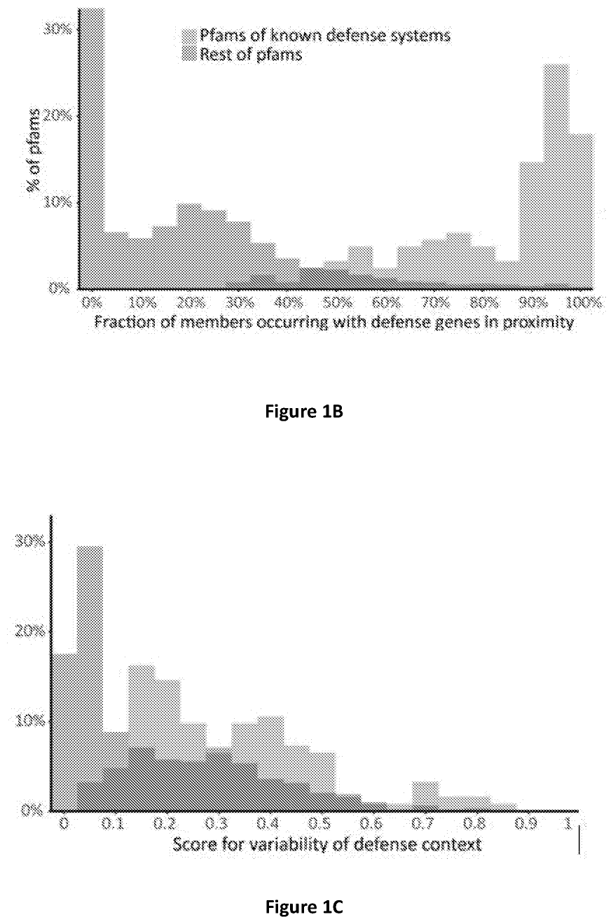

A non-limiting example of a method of identifying a defense system, which can be used according to embodiments disclosed herein, is described at least in the Materials and Methods hereinbelow, Example 1, Example 2, and in FIG. 1, which is to be understood as forming an integral part of the present section.

Once selected, the identified cassette of genes comprising said gene with conserved synteny in said plurality of prokaryotic genomes can be cloned or synthesized.

The gene(s) can be ligated into expression constructs and used in a variety of applications e.g., for conferring phage resistance in for example, but not limited to the food, feed, medical and veterinary industries.

In some embodiments, an identified cassette of gene confers phage resistance to a starter culture. In some embodiments, an identified cassette of gene confers phage resistance to a food product. In some embodiments, a food product comprises a dairy product, an alcoholic beverage, or a natto. In some embodiments, an identified cassette of gene confers phage resistance to a dairy product. In some embodiments, an identified cassette of gene confers phage resistance to an alcoholic beverage. In some embodiments, an identified cassette of gene confers phage resistance to a natto.

In some embodiments, an identified cassette of gene confers phage resistance to a milk product. In some embodiments, an identified cassette of gene confers phage resistance to milk. In some embodiments, an identified cassette of gene confers phage resistance to a sour milk. In some embodiments, an identified cassette of gene confers phage resistance to a milk butter milk. In some embodiments, an identified cassette of gene confers phage resistance to a cheese. In some embodiments, an identified cassette of gene confers phage resistance to a yogurt. In some embodiments, an identified cassette of gene confers phage resistance to a viili. In some embodiments, an identified cassette of gene confers phage resistance to yakult. In some embodiments, an identified cassette of gene confers phage resistance to a casein. In some embodiments, an identified cassette of gene confers phage resistance to a wine. In some embodiments, an identified cassette of gene confers phage resistance to a sake. In some embodiments, an identified cassette of gene confers phage resistance to a food additive. In some embodiments, an identified cassette of gene confers phage resistance to a feed. In some embodiments, an identified cassette of gene confers phage resistance to a nutritional supplement. In some embodiments, an identified cassette of gene confers phage resistance to a probiotic supplement. In some embodiments, an identified cassette of gene confers phage resistance to a health care product. In some embodiments, an identified cassette of gene confers phage resistance to an antibiotic. In some embodiments, an identified cassette of gene confers phage resistance to polymyxin. In some embodiments, an identified cassette of gene confers phage resistance to a colistin. In some embodiments, an identified cassette of gene confers phage resistance to a bacitracin. In some embodiments, an identified cassette of gene confers phage resistance to a personal care product. In some embodiments, an identified cassette of gene confers phage resistance to a veterinary product.

### Antagonists of Defense Systems and Methods of Use Thereof

In some embodiments, there is provided an antagonist of a defense system disclosed herein, an “anti-defense system agent”. A skilled artisan would appreciate that the terms “anti-defense system agent” and “anti-Defense System agent” and “agent” may in some embodiments, be used interchangeably having all the same elements and qualities. In some embodiments, an anti-defense system agent may antagonize more than one Defense System function. In some embodiments, an anti-defense system agent may antagonize more than one Defense System function, selected from Defense systems Ia-Xc. In some embodiments, an anti-Defense System agent may antagonize more than one Defense System. In some embodiments, an anti-Defense System agent may antagonize more than one Defense System selected from Defense systems Ia-Xc.

In some embodiments, an anti-Defense System agent is capable of down regulating expression and/or activity of at least one Defense System component. In some embodiments, an anti-Defense System agent is capable of down regulating expression and/or activity of at least one Defense System component, wherein said Defense System is selected from Defense Systems Ia-Xc.

In some embodiments, the anti-defense system agent is directed against a defense system component comprising an enzyme activity, for example but not limited to a HamB polypeptide, a SduA polypeptide, a GajA polypeptide, a GajB polypeptide, a PtuA polypeptide, a PtuB polypeptide, a LmuB polypeptide, a ZorD polypeptide, a ZorE polypeptide, a DruE polypeptide, a DruM polypeptide, or a JetC polypeptide. For example, the anti-defense agent may interfere with the expression of an active defense system component or, for example in its DNA methyltransferase function, helicase function, exonuclease function, or endonuclease function. For example, in some embodiments, a methylase activity may be inhibited by the use of common inhibitors of such an enzyme e.g., 5-Azacytidine, Decitabine Zebularine, RG108, Hydralazine hydrochloride, and Psammaplin A.

In some embodiments, an at least one component comprises a component selected from Defense Systems Ia, Ib, II, IIIa, IIIb, IIIc, IV, V, VI, VII, VIII, IX, Xa, Xb, and Xc. In some embodiments, a component comprises a functional component. In some embodiments, a component comprises an essential component. In some embodiments, a component comprises the full-length component. In some embodiments, a component comprises a functional portion of the component.

The components of defense systems comprising a Defense System Ia-Xc and combinations thereof, have been described in detail above. In some embodiments, polypeptide components of these systems include but are not limited to ZorA, ZorB, ZorC, ZorD, ZorE (Defense System Ia and Ib); ThsA and ThsB (Defense System II); DruA, DruB, DruC, DruD, DruE, DruH, DruM, DruG, DruF (Defense Systems IIIa, IIIb, IIIc); HamA and HamB (Defense System IV); SduA (Defense System V); GajA and GajB (Defense System VI); PtuA and PtuB (Defense System VII); LmuA and LmuB (Defense System VIII); KwaA and KwaB (Defense System IX); and JetA, JetB, JetC, and JetD, and variants thereof (Defense Systems Xa, Xb, Xc). In some embodiments, polynucleotide (e.g., gene) components, include but are not limited to zorA, zorB, zorC, zorD, zorE (Defense Systems Ia, Ib); thsA and thsB (Defense System II); druA, druB, druC, drub, druE, druF, druG, druH, druM (Defense Systems IIIa, IIIb, IIIc); hamA and hamB (Defense System IV); sduA (Defense System V); gajA and gajB (Defense System VI); ptuA and ptuB (Defense System VII); lmuA and lmuB (Defense System VIII); kwaA and kwaB (Defense System IX); and jetA, jetB, jetC, and jetD and variants thereof (Defense Systems Xa, Xb, Xc). In some embodiments, a component comprises a component homolog. Defense System components, nucleic acid sequences thereof, and amino acid sequences thereof are presented herein in Table 18.

In some embodiments, a Defense System described herein comprises at least two different polypeptide components. The polypeptide components of the Defense Systems described herein, and the nucleic acids encoding them are described in detail above, and in Tables 8-18. In some embodiments, a Defense System described herein comprises at least three different polypeptide components. In some embodiments, a Defense System described herein comprises at least four different polypeptide components. In some embodiments, a Defense System described herein comprises at least five different polypeptide components. In some embodiments, a Defense System described herein comprises multiple polypeptide components, wherein the components comprise different polypeptides. In some embodiments, a Defense System described herein comprises multiple polypeptide components, wherein the components comprise different polypeptides and duplications of a same polypeptide. In some embodiments, components comprising duplication of a same polypeptide comprise homologues of that polypeptide.

In some embodiments, a Defense System comprising at least two different polypeptide components provides a functional defense activity (protection from foreign nucleic acid) for a cell. In some embodiments, a Defense System described herein comprises at least three different polypeptide components provides a functional defense activity (protection from foreign nucleic acid) for a cell. In some embodiments, a Defense System described herein comprises at least four different polypeptide components provides a functional defense activity (protection from foreign nucleic acid) for a cell. In some embodiments, a Defense System described herein comprises at least five different polypeptide components provides a functional defense activity (protection from foreign nucleic acid) for a cell. In some embodiments, a Defense System described herein comprises multiple polypeptide components, wherein the components comprise different polypeptides, and wherein the Defense System provides a functional defense activity (protection from foreign nucleic acid) for a cell. In some embodiments, a Defense System described herein comprises multiple polypeptide components, wherein the components comprise different polypeptides and duplications of a same polypeptide, wherein said Defense System provides a functional defense activity (protection from foreign nucleic acid) for a cell. In some embodiments, components comprising duplication of a same polypeptide comprise homologues of that polypeptide.

In some embodiments, an agent is capable of down regulating expression and/or activity of at least two components of a defense system. In some embodiments, an agent is capable of down regulating at least one components of at least two different Defense Systems. In some embodiments, an agent is capable of down regulating expression and/or activity of at least two components of a Defense System. In some embodiments, an agent is capable of down regulating at least two components of the same Defense System. In some embodiments, an agent is capable of down regulating expression and/or activity of at least two components of a Defense System selected from the components of Defense Systems Ia-Xc.

In some embodiments, an agent is capable of down regulating expression and/or activity of 1-40 components of a defense system. In some embodiments, an agent is capable of down regulating expression and/or activity of 1-4 components of a defense system. In some embodiments, an agent is capable of down regulating expression and/or activity of 1-5 components of a defense system. In some embodiments, an agent is capable of down regulating expression and/or activity of 1-10 components of a defense system. In some embodiments, an agent is capable of down regulating expression and/or activity of 10-20 components of a defense system. In some embodiments, an agent is capable of down regulating expression and/or activity of 20-30 components of a defense system. In some embodiments, an agent is capable of down regulating expression and/or activity of 30-40 components of a defense system. In some embodiments, an agent is capable of down regulating expression and/or activity of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43 components of a defense system. In some embodiments, an agent is capable of down regulating at least one components of at least two different Defense Systems. In some embodiments, an agent is capable of down regulating expression and/or activity of at least two components of a Defense System. In some embodiments, an agent is capable of down regulating at least two components of the same Defense System. In some embodiments, an agent is capable of down regulating expression and/or activity of at least two components of a Defense System selected from the components of Defense Systems Ia-Xc.

A skilled artisan would recognize that the phrase “anti-Defense agent” encompasses an agent capable of specifically inhibiting the expression of a target Defense System gene (at the DNA or RNA level) or alternatively specifically impairing the functionality of the target Defense System protein. In some embodiments, an agent is capable of specifically inhibiting the expression of a target Defense System component (at the DNA or RNA level) or alternatively specifically impairing the functionality of the target Defense System component polypeptide.

In some embodiments, the anti-Defense system agent is directed against one of the Defense System components. In some embodiments, the anti-Defense System agent is directed against multiple components of a Defense System. In some embodiments, the anti-Defense System agent is directed against at least 2 components of a Defense System. In some embodiments, the anti-Defense System agent is directed against at least 3 components of a Defense System. In some embodiments, the anti-Defense System agent is directed against at least 4 components of a Defense System. In some embodiments, the anti-Defense System agent is directed against at least 5 components of a Defense System.

In some embodiments, the anti-Defense system agent is directed against any of the components disclosed in Table 18. In some embodiments, the anti-Defense system agent is directed against any combination of the components disclosed in Table 18. In some embodiments, the combination of components may be from a same Defense System selected from Defense Systems Ia-Xc. In some embodiments, the combination of components may be from different Defense Systems selected from Defense Systems Ia-Xc. In some embodiments, the combination of components may be from the same and different Defense Systems selected from Defense Systems Ia-Xc.

In some embodiments, the agent is directed against all of the components of a single Defense System. In some embodiments, the agent is directed against all of the essential components of a single Defense System. In some embodiments, the agent is directed against all of the components of a single Defense System selected from Defense Systems Ia-Xc. In some embodiments, the agent is directed against all of the essential components of a single Defense System, selected from Defense Systems Ia-Xc.

Down regulation of a Defense System can be effected on the genomic and/or the transcript level using a variety of molecules that interfere with transcription and/or translation (e.g., RNA silencing agents), or on the protein level using e.g., aptamers, small molecules and inhibitory peptides, antagonists, enzymes that cleave the polypeptide and the like.

In some embodiments the anti-Defense System agent is selected from the group consisting of a nucleic acid suitable for silencing expression, aptamers, small molecules and inhibitory peptides.

A skilled artisan would appreciate that the phrase “nucleic acid suitable for silencing expression” refers to regulatory mechanisms mediated by nucleic acid molecules, which result in the inhibition or “silencing” of the expression of a corresponding protein-coding gene.

Methods for qualifying efficacy and detecting inhibition or “silencing” of the expression of a corresponding protein-coding gene are well known in the art and include, but are not limited to, DNA sequencing, electrophoresis, an enzyme-based mismatch detection assay and hybridization assay such as PCR, RT-PCR, RNase protection, in-situ hybridization, primer extension, Southern blot, Northern Blot and dot blot analysis. Inhibition or “silencing” of the expression of a specific protein-coding gene can also be determined at the protein level using e.g. chromatography, electrophoretic methods, immunodetection assays such as ELISA and Western blot analysis or immunohistochemistry. In addition, one ordinarily skilled in the art can readily design a knock-in/knock-out construct including positive and/or negative selection markers for efficiently selecting transformed cells that e.g. underwent a homologous recombination event with the construct.

In some embodiments, downregulation can be achieved by DNA silencing.

Methods of introducing nucleic acid alterations to a gene of interest are well known in the art [see for example Menke D. Genesis (2013) 51: 618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases.

Non-limiting examples of DNA silencing agents that can be used In some embodiments include: the “Hit and run” or “in-out” strategy; the “double-replacement” or “tag and exchange” strategy; site-specific recombinases such as, but not limited to, cre recombinase and Flp recombinase; genome editing using engineered endonucleases such as, but not limited to, meganucleases, Zinc finger nucleases (ZFNs), transcription-activator like effector nucleases (TALENs) and CRISPR/Cas system; transposases; and genome editing using recombinant adeno-associated virus (rAAV) platform.

It will be appreciated that the agent can be a mutagen that causes random mutations and the cells exhibiting downregulation of the expression level and/or activity of a component or components of a Defense System disclosed herein, for example but not limited to the components and Defense Systems disclosed in Table 18. The mutagens may be, but are not limited to, genetic, chemical or radiation agents.

Downregulation can also be achieved by RNA silencing.

In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In some embodiments, the RNA silencing agent is capable of inducing RNA interference. In some embodiments, the RNA silencing agent is capable of mediating translational repression.

In some embodiments, the RNA silencing agent is specific to the target RNA of a component of a Defense System disclosed herein, and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

Non-limiting examples of RNA silencing agents that can be used in some embodiments include but are not limited to antisense, DsRNA, siRNA, shRNA, miRNA and miRNA mimics.

Numerous methods are known in the art for gene silencing particularly in prokaryotes; examples include but are not limited to U.S. Patent Application 20040053289 which teaches the use of si hybrids to down-regulate prokaryotic genes, and U.S. Patent Application PCT/US09/69258 which teaches the use of CRISPR to downregulate prokaryotic genes.

In some embodiments, the inhibition can be carried out at the protein level which interferes with protein activity, such as by the use of aptamers. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

In some embodiments, a small molecule, peptides or antibodies can be used which interfere with the Defense System component(s) protein function (e.g., catalytic or interaction).

In some embodiments, an anti-Defense agent comprises any molecule which binds to and/or cleaves a Defense System component gene or component polypeptide. Such molecules can be, for example, a small molecule, antagonists, or inhibitory peptide.

It will be appreciated that a non-functional analogue of at least a catalytic or binding portion of a Defense System polypeptide component can be also used as an anti-Defense System agent.

In some embodiments, there is provided a method of inducing phage sensitivity in bacteria, the method comprising introducing the agent disclosed herein into bacteria, which expresses a defense system having an anti-phage activity, said system comprising at least one component selected from the group consisting of said component polypeptides of said Defense System, thereby inducing sensitivity of the bacteria to phage infection.

In some embodiments, there is provided a method of killing bacteria, the method comprising introducing into bacteria, which expresses a defense system having an anti-phage activity, as described herein, an anti-defense system agent capable of down regulating expression and/or activity of a component polypeptide of the defense system, thereby killing the bacteria.

In another embodiment, there is provided an isolated anti-defense system agent capable of down regulating expression and/or activity of at least two defense system components. In some embodiments, the bacteria express the at least one Defense System component for which the anti-Defense System agent is directed against.

In some embodiments, the method further comprises infecting said bacteria with a phage.

Specifically, introducing an anti-Defense System agent is effected such that the positioning of the anti-Defense System agent is in direct or indirect contact with the bacterial cell. In some embodiments, both applying the anti-Defense System agent or agents disclosed herein to a desirable surface and/or directly to the bacterial cells. In some embodiments, the surface is comprised in a biological tissue, such as for example, mammalian tissues e.g. the skin.

It will be appreciated that the bacteria may be comprised inside a particular organism, (e.g. intracellularly or extracellularly) for example inside a mammalian body or inside a plant. In this case, the introducing may be effected by administering the anti-Defense System agent per se or by transfecting the cells of the organism with the anti-Defense System agents.

In some embodiments, introducing with an anti-Defense System agent is effected in-vivo. In some embodiments, introducing with an anti-Defense System agent is effected ex-vivo. In some embodiments, introducing with an anti-Defense System agent is effected in-vitro.

In some embodiments, there is provided isolated bacteria generated by introducing into bacteria an anti-Defense System agent as described above. In some embodiments, there is provided isolated bacteria generated by introducing into the bacteria an anti-Defense System agent in-vitro or ex-vivo.

Thus, following the above teachings there is provided isolated bacteria comprising the anti-Defense System agent.

According to some embodiments, a Defense System or an anti-Defense System agent is provided in a formulation suitable for cell penetration that enhances intracellular delivery of the system or the agent.

Any suitable penetrating agent for enhancing penetration of a Defense System or its components or an anti-Defense System agent to cell (e.g., bacteria) may be used, as known by those of skill in the art. Examples include but are not limited to:

Phages—Phages offer several advantages including lateral infection, higher efficiency of transformation, and targeting to, and propagation in, specific bacteria.

Cell-Penetrating Peptides (CPPs)—CPPs, for example TAT (transcription activator from HIV-1) are short peptides (≤40 amino acids), with the ability to gain access to the interior of almost any cell. They are highly cationic and usually rich in arginine and lysine amino acids. They have the exceptional property of carrying into the cells a wide variety of covalently and noncovalently conjugated cargoes such as proteins, oligonucleotides, and even 200 nm liposomes. Protocols for producing CPPs-cargos conjugates and for infecting cells with such conjugates can be found, for example L. Theodore et al. [The Journal of Neuroscience, (1995) 15(11): 7158-7167], Fawell S, et al. [Proc Natl Acad Sci USA, (1994) 91:664-668], and Jing Bian et al. [Circulation Research. (2007) 100: 1626-1633].

The expression level and/or activity level of the Defense System components expressed in the cells of some embodiments can be determined using methods known in the arts, e.g. but not limited to selectable marker gene, Northern blot analysis, PCR analysis, DNA sequencing, RNA sequencing, Western blot analysis or Immunohistochemistry.

In some embodiments, there is provided a method of treating bacterial infection in a subject in need thereof, the method comprising administering to the subject a therapeutically effective amount of the anti-Defense System agent, thereby treating the bacterial infection in the subject. In some embodiments, there is provided the agent, for use in the treatment of bacterial infection in a subject in need thereof.

A skilled artisan would appreciate that the term “treating” encompasses in some embodiments, curing, reversing, attenuating, alleviating, minimizing, suppressing or halting the deleterious effects of a pathogen infection. Those of skill in the art will understand that various methodologies and assays can be used to assess the development of the pathology, and similarly, various methodologies and assays may be used to assess the reversal, attenuation, alleviation or suppression of the pathology.

In some embodiments, a “subject in need thereof” includes but is not limited to mammals. In some embodiments, the subject comprises a human being of any gender and at any age which suffer from pathogen infection.

The anti-Defense System agent may be used alone or together with additional antimicrobial agents (e.g. phage therapy, antibiotic and/or additional anti-microbial peptides).

In some embodiments, a method disclosed herein further comprises administering to the subject a phage therapy and/or an antibiotic. In some embodiments, the uses further comprise a phage therapy and/or an antibiotic.

In some embodiments, antibiotics include, but are not limited to aminoglycoside antibiotics, cephalosporins, quinolone antibiotics, macrolide antibiotics, penicillin, sulfonamides, tetracyclines and carbapenems. It will be appreciated that since the agents disclosed herein, enhance the anti-bacterial effect of the antibiotic, doses of the antibiotic may be lower (e.g. 20% lower, 30% lower, 40% lower, 50% lower, 60% lower, 70% lower, 80% lower or even 90% lower) than those currently in use.

In the context of manufacturing of food products and especially fermented products in the food industry, an anti-Defense System agent can be used to eliminate spoilage bacteria or the end product from traces of used bacteria.

Thus, in some embodiments, there is provided a method of preparing a food, food additive, feed, nutritional supplement, probiotic supplement, personal care product, health care product and veterinary product, the method comprising adding to the food, food additive, feed, nutritional supplement, probiotic supplement, personal care product, health care product and veterinary product the anti-Defense System agent, thereby preparing the food, food additive, feed, nutritional supplement, probiotic supplement, personal care product, health care product and veterinary product.

Following the above teachings there is provided a food, food additive, feed, nutritional supplement, probiotic supplement, personal care product, health care product and veterinary product comprising the anti-Defense System agent.

The nucleic acid sequences, constructs, transmissible elements, polypeptides and cells comprising the Defense System components or the anti-Defense System agent of some embodiments, can be administered to a starter culture, a fermentation vat or an organism per se, or in a composition where it is mixed with suitable carriers or excipients.

The phrase “pharmaceutical composition” as used herein refers to a preparation of one or more of the active ingredients described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

As used herein the term “active ingredient” refers to any one of the component polypeptides or polynucleotides of a Defense System, an anti-Defense System agent, or cells generated according to the methods disclosed herein, accountable for the biological effect.

Techniques for formulation and administration of drugs may be found in the latest edition of “Remington's Pharmaceutical Sciences,” Mack Publishing Co., Easton, Pa., which is herein fully incorporated by reference and are further described herein below.

It will be appreciated that the polypeptides, polynucleotides, nucleic acid construct(s) or other agents can be provided to the individual with additional active agents to achieve an improved therapeutic effect as compared to treatment with each agent by itself.

In some embodiments, additional agents include phage therapy and antibiotics (e.g. rifampicin, chloramphenicol and spectinomycin).

In some embodiments, the composition further comprises a phage.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the disclosure herein, may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the disclosure herein are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation may also be formulated in rectal compositions such as suppositories or retention enemas, using, e.g., conventional suppository bases such as cocoa butter or other glycerides.

The preparation may also be formulated as a topical composition, such as a spray, a cream, a mouthwash, a wipe, a foam, a soap, an oil, a solution, a lotion, an ointment, a paste, a gel and a patch.

Pharmaceutical compositions suitable for use in context include compositions wherein the active ingredients are contained in an amount effective to achieve the intended purpose. More specifically, a therapeutically effective amount means an amount of active ingredients effective to prevent, alleviate or ameliorate symptoms of disease (e.g., bacterial infection) or prolong the survival of the subject being treated.

Determination of a therapeutically effective amount is well within the capability of those skilled in the art.

For any preparation used in the methods disclosed herein, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on e.g. the subject being treated, the severity of the affliction, the manner of administration, the judgment of the prescribing physician, etc.

Compositions of some embodiments, may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation formulated in a compatible pharmaceutical carrier may also be prepared, placed in an appropriate container, and labeled for treatment of an indicated condition, as is further detailed above.

In some embodiments, there is provided an article of manufacture or a kit identified for treating bacterial infection comprising the anti-Defense System agent; and a phage and/or an antibiotic. In some embodiments, the anti-Defense System agent and the phage and/or the antibiotic are packaged in separate containers. In some embodiments, the anti-Defense System agent and the phage and/or the antibiotic are in co-formulation.

In some embodiments, there is provided a method of screening for identifying phage useful for infecting bacteria, the method comprising:

(a) contacting a phage with bacteria expressing a Defense System; and

(b) determining deleterious effects induced in said bacteria, wherein an increase in deleterious effects of said bacteria in the presence of said phage compared to deleterious effects in the absence of said phage is indicative of a phage useful for infecting said bacteria.

In some embodiments, the method comprising further isolating the phage, characterizing it in terms of sequencing and compatibility with phages species and the ability to infect different bacterial species.

### Use of a Defense System or Components Thereof, for Gene Editing

In some embodiments, disclosed herein is a method of gene editing, the method comprising contacting a nucleic acid sequence with a defense system component, as described herein, wherein said contact edits a gene sequence within the nucleic acid. In some embodiments, said editing comprises deleting a gene, deleting a portion of a gene, deleting upstream regulatory sequences of a gene, deleting downstream regulatory sequences of a gene, deleting upstream and downstream regulatory sequences of a gene, repairing a gene sequence, replacing a gene, replacing a part of a gene, replacing an upstream sequence of a gene, replacing a downstream sequence of a gene, replacing an upstream and a downstream sequence of a gene, modifying a gene sequence, modifying a sequence upstream of a gene, modifying a sequence downstream of a gene, modifying a sequence upstream and downstream of a gene, or a combination thereof.

In some embodiments, use of a defense system component, as described herein, for gene editing provides greater precision in editing a nucleotide sequence. In some embodiments, gene editing provides greater precision of cutting the nucleotide sequence at a cleavage site. In some embodiments, gene editing comprising cutting the nucleic acid sequence at a specific cleavage site. In some embodiments, gene editing comprising cutting the nucleic acid sequence based on the modification of nucleotides within the nucleic acid. In some embodiments, gene editing comprising inhibition of cutting the nucleic acid sequence based on the modification of nucleotides within the nucleic acid. In some embodiments, cleavage comprises cleavage of the phosphodiester bond within a nucleotide sequence. In some embodiments, cleavage is ATP dependent.

In some embodiments, use of a defense system component, as described herein, for gene editing provides separation of two annealed nucleic acid strands. In some embodiments, gene editing comprises separation of double stranded DNA. In some embodiments, gene editing comprises separation of double stranded RNA. In some embodiments, gene editing comprises separation of a double stranded RNA-DNA hybrid. In some embodiments, separation uses energy derived from ATP hydrolysis. In some embodiments, separation of a double stranded sequence is based on the specific sequence of the double stranded sequence. In some embodiments, separation of a double stranded sequence is based on the methylation pattern within the double stranded sequence. In some embodiments, separation of a double stranded sequence is based on the modification pattern within the double stranded sequence.

In some embodiments, use of a defense system component, as described herein, for gene editing provides unwinding of a double stranded nucleic acid sequence one base at a time. In some embodiments, unwinding is of a double stranded DNA. In some embodiments, unwinding is of a double stranded RNA. In some embodiments, unwinding is of a hybrid double stranded RNA-DNA. In some embodiments, unwinding uses energy derived from ATP hydrolysis. In some embodiments, unwinding of a double stranded sequence is based on the specific sequence of the double stranded sequence. In some embodiments, unwinding of a double stranded sequence is based on the methylation pattern within the double stranded sequence. In some embodiments, unwinding of a double stranded sequence is based on the modification pattern within the double stranded sequence.

In some embodiments, use of a defense system component, as described herein, for gene editing provides cleaving nucleotides one at a time from the end of a polynucleotide chain. In some embodiments, the polynucleotide change comprises DNA. In some embodiments, the polynucleotide chain comprises RNA. In some embodiments, the polynucleotide chain comprises a hybrid RNA-DNA construct. In some embodiments, cleavage is based on the specific sequence of the double stranded sequence. In some embodiments, cleavage is based on the methylation pattern within the double stranded sequence. In some embodiments, cleavage is based on the modification pattern within the double stranded sequence.

In some embodiments, use of a defense system component, as described herein, for gene editing provides overwinding or underwinding of DNA. In some embodiments, the overwinding or underwinding comprises cleavage of the phosphate backbone of one of the DNA strands. In some embodiments, the overwinding or underwinding comprises cleavage of the phosphagen backbone of both of the DNA strands. In some embodiments, overwinding or underwinding is based on the specific sequence of the double stranded sequence. In some embodiments, overwinding or underwinding is based on the methylation pattern within the double stranded sequence. In some embodiments, overwinding or underwinding is based on the modification pattern within the double stranded sequence.

In some embodiments, modifications comprise methylation and acetylation. In some embodiments, methylation comprises methylation of adenine bases. In some embodiments, methylation comprises methylation of cytosine bases.

In some embodiments, gene editing comprises cutting the nucleic acid sequence based on the methylation pattern of the nucleic acid. In some embodiments, gene editing comprising cutting the nucleic acid sequence based on the lack of methylation of the nucleic acid. In some embodiments, gene editing comprises inhibition of cutting the nucleic acid sequence based on the methylation pattern of the nucleic acid. In some embodiments, gene editing comprising inhibition of cutting the nucleic acid sequence based on the lack of methylation of the nucleic acid.

A skilled artisan would appreciate that the terms “cutting” and “cleaving” may be used interchangeably in some embodiments, having all the qualities and meanings.

In some embodiments, cutting a nucleic acid sequence comprises cleavage of a single stranded nucleic acid. In some embodiments, cutting a nucleic acid sequence comprises cleavage of a double stranded nucleic acid. In some embodiments, cutting a nucleic acid sequence comprises cleavage of a DNA molecule. In some embodiments, cutting a nucleic acid sequence comprises cleavage of an RNA molecule.

In some embodiments, cutting a nucleic acid sequence comprises recognizing a specific nucleotide sequence. In some embodiments, cutting a nucleic acid sequence comprises recognizing a specific pattern of methylation of a nucleotide sequence. In some embodiments, editing comprising inhibiting cleavage of a nucleic acid sequence, wherein said inhibition of cleavage comprises recognizing a specific pattern of methylation of the nucleotide sequence.

In some embodiments, the nucleotide sequence being edited is comprised in a cell. In some embodiments, the nucleotide sequence being edited is not comprised within a cell. In some embodiments, editing of a nucleotide sequence within a cell, comprises genetically engineering the cell.

In some embodiments, genetic engineering of a cell comprises deleting a gene, deleting a portion of a gene, deleting upstream regulatory sequences of a gene, deleting downstream regulatory sequences of a gene, deleting upstream and downstream regulatory sequences of a gene, repairing a gene sequence, replacing a gene, replacing a part of a gene, replacing an upstream sequence of a gene, replacing a downstream sequence of a gene, replacing an upstream and a downstream sequence of a gene, modifying a gene sequence, modifying a sequence upstream of a gene, modifying a sequence downstream of a gene, modifying a sequence upstream and downstream of a gene, or a combination thereof, within the cell.

In some embodiments, a cell comprises a eukaryotic cell. In some embodiments, a cell comprises a prokaryotic cell. In some embodiments, a cell comprises a plant cell. In some embodiments, a cell comprises a yeast cell. In some embodiments, a cell comprises a protozoa cell.

In some embodiments, genetic engineering of a cell provides cells useful in treating a disease.

In some embodiments, genetic engineering of a plant cell provides for improved plant growth. In some embodiments, genetic engineering of a plant cell provides for improved plant growth under adverse conditions, including but not limited to extreme heat, extreme cold, drought, high salt content, lack of nutrients, or any combination thereof. In some embodiments, genetic engineering of a plant cell provides for higher crop yield. In some embodiments, genetic engineering of a plant cell provides for higher crop yield under adverse conditions, including but not limited to extreme heat, extreme cold, drought, high salt content, lack of nutrients, or any combination thereof.

In some embodiments, gene editing provides improved identification and validation of therapeutic agents. In some embodiments, gene editing provides improved development and production of animal models of human disease. In some embodiments, uses of gene editing leading to improvements comprises performing the work in a shorter amount of time and with more precision.

In some embodiments, a method of gene editing a nucleic acid sequence comprises contacting the nucleic acid sequence with a component of a defense system as described herein, thereby editing the nucleic acid sequence. In some embodiments, a method of cutting a nucleic acid sequence comprises contacting the nucleic acid sequence with a component of a defense system as described herein, thereby cutting the nucleic acid sequence.

Components of defense systems have been described in detail here (Tables 7-17 and 18).

In some embodiments, a method of editing a nucleic acid sequence comprises contacting the nucleic acid sequence with a component of a Defense System Ia, said component comprising a ZorA polypeptide, a ZorB polypeptide, a ZorC polypeptide, or a ZorD polypeptide, thereby editing the nucleic acid sequence.

In some embodiments, a method of editing a nucleic acid sequence comprises contacting the nucleic acid sequence with a component of a Defense System Ib, said component comprising a ZorA polypeptide, a ZorB polypeptide, or a ZorD polypeptide, thereby editing the nucleic acid sequence.

In some embodiments, a method of editing a nucleic acid sequence comprises contacting the nucleic acid sequence with a component of a Defense System II, said component comprising a ThsA polypeptide or a ThsB polypeptide, thereby editing the nucleic acid sequence.

In some embodiments, a method of editing a nucleic acid sequence comprises contacting the nucleic acid sequence with a component of a Defense System IIIa, said component comprising a DruA polypeptide, a DruB polypeptide, a DruC polypeptide, a DruD polypeptide, or a DruE polypeptide, thereby editing the nucleic acid sequence.

In some embodiments, a method of editing a nucleic acid sequence comprises contacting the nucleic acid sequence with a component of a Defense System IIIb, said component comprising a DruM polypeptide, a DruF polypeptide, a DruG polypeptide, or a DruE polypeptide, thereby editing the nucleic acid sequence.

In some embodiments, a method of editing a nucleic acid sequence comprises contacting the nucleic acid sequence with a component of a Defense System IIIc, said component comprising a DruH polypeptide, or a DruE polypeptide, thereby editing the nucleic acid sequence.

In some embodiments, a method of editing a nucleic acid sequence comprises contacting the nucleic acid sequence with a component of a Defense System IV, said component comprising a HamA polypeptide or a HamB polypeptide, thereby editing the nucleic acid sequence.

In some embodiments, a method of editing a nucleic acid sequence comprises contacting the nucleic acid sequence with a component of a Defense System V, said component comprising a SduA polypeptide, thereby editing the nucleic acid sequence.

In some embodiments, a method of editing a nucleic acid sequence comprises contacting the nucleic acid sequence with a component of a Defense System VI, said component comprising a GajA polypeptide or a GajB polypeptide, thereby editing the nucleic acid sequence.

In some embodiments, a method of editing a nucleic acid sequence comprises contacting the nucleic acid sequence with a component of a Defense System VII, said component comprising a PtuA polypeptide or a PtuB polypeptide, thereby editing the nucleic acid sequence.

In some embodiments, a method of editing a nucleic acid sequence comprises contacting the nucleic acid sequence with a component of a Defense System VIII, said component comprising a LmuA polypeptide or a LmuB polypeptide, thereby editing the nucleic acid sequence.

In some embodiments, a method of editing a nucleic acid sequence comprises contacting the nucleic acid sequence with a component of a Defense System IX, said component comprising a KwaA polypeptide or a KwaB polypeptide, thereby editing the nucleic acid sequence.

In some embodiments, a method of editing a nucleic acid sequence comprises contacting the nucleic acid sequence with a component of a Defense System Xa, said component comprising a JetA polypeptide, a JetB polypeptide, a JetC polypeptide, or a JetD polypeptide, thereby editing the nucleic acid sequence.

In some embodiments, a method of editing a nucleic acid sequence comprises contacting the nucleic acid sequence with a component of a Defense System Xb, said component comprising a JetAⁱⁱ polypeptide, a JetBⁱⁱ polypeptide, a JetCⁱⁱ polypeptide, or a JetDⁱⁱ polypeptide, thereby editing the nucleic acid sequence.

In some embodiments, a method of editing a nucleic acid sequence comprises contacting the nucleic acid sequence with a component of a Defense System Xc, said component comprising a JetAⁱⁱⁱ polypeptide, a JetBⁱⁱⁱ polypeptide, a JetCⁱⁱⁱ polypeptide, or a JetDⁱⁱ polypeptide, thereby editing the nucleic acid sequence.

In some embodiments, a method of editing a nucleic acid sequence comprises editing said sequence within a cell. One skilled in the art would appreciate that a method of editing a nucleic acid sequence within a cell comprises in some embodiments, expressing a component of a defense system in the cell. Genes encoding the components of defense systems described herein, have been described in detail (Tables 7-17 and 18).

In some embodiments, contacting the nucleic acid sequence with the defense system component can be performed by any in vitro conditions including for example, adding a defense system polypeptide to a naked nucleic acid sequence, adding a defense system polypeptide to a cell expressing the nucleic acid sequence, or introducing a defense system gene nucleotide sequence into a cell and expressing the gene sequence, such that the defense system component is in or comes into direct contact with the nucleic acid sequence to be edited.

The contacting may be effected with at least 1, at least 2, at least 3, or at least 4 defense system components.

In some embodiments, the contacting is effected with a ZorA, ZorB, ZorC, or ZorD polypeptide, or any combination thereof. In some embodiments, the contacting is effected with a ZorA, ZorB, or ZorE polypeptide, or any combination thereof. In some embodiments, the contacting is effected with a ThsA or a ThsB polypeptide, or any combination thereof. In some embodiments, the contacting is effected with a DruA, DruB, DruC, DruD, or DruE polypeptide, or any combination thereof. In some embodiments, the contacting is effected with a DruM, DruF, DruG, or DruE polypeptide, or any combination thereof. In some embodiments, the contacting is effected with a DruH or a DruE polypeptide, or any combination thereof. In some embodiments, the contacting is effected with a HamA or a HamB polypeptide, or any combination thereof. In some embodiments, the contacting is affected with a SduA polypeptide, or any combination thereof. In some embodiments, the contacting is effected with a GajA or a GajB polypeptide, or any combination thereof. In some embodiments, the contacting is effected with a PtuA or a PtuB polypeptide, or any combination thereof. In some embodiments, the contacting is affected with a Lmu or a LmuB polypeptide, or any combination thereof. In some embodiments, the contacting is effected with a KwaA or a KwaB polypeptide, or any combination thereof. In some embodiments, the contacting is effected with a JetA, JetB, JetC, or JetD polypeptide, or any combination thereof. In some embodiments, the contacting is effected with a JetAⁱⁱ, JetBⁱⁱ, JetCⁱⁱ, or JetDⁱⁱ polypeptide, or any combination thereof. In some embodiments, the contacting is effected with a JetAⁱⁱⁱ, JetBⁱⁱⁱ, JetCⁱⁱⁱ, or JetDⁱⁱⁱ polypeptide, or any combination thereof.

In some embodiments, the nucleic acid sequence being edited is comprised in a cell. In some embodiments, the nucleic acid sequence being edited is comprised in a cell that is not expressing an endogenous defense system or components thereof. In some embodiments, the nucleic acid sequence being edited is comprised in a cell that is not expressing an endogenous functional defense system or components thereof.

In some embodiments, the nucleic acid sequence being edited is not comprised in a cell expressing an endogenous defense system or components thereof. In some embodiments, the nucleic acid sequence being edited is not comprised in a cell expressing an endogenous functional defense system or components thereof.

In some embodiments, wherein the nucleic acid being edited is comprised in a cell expressing a component polypeptide of a defense system, the method further comprises introducing into the cell an agent capable of downregulating expression and/or activity of the expressed component polypeptide.

In some embodiments, a method of editing a nucleic acid sequence within a cell comprises expressing a component of a Defense System Ia within said cell, said component comprising a ZorA polypeptide, a ZorB polypeptide, a ZorC polypeptide, or a ZorD polypeptide, thereby editing the nucleic acid sequence.

In some embodiments, a method of editing a nucleic acid sequence within a cell comprises expressing a component of a Defense System Ib within said cell, said component comprising a ZorA polypeptide, a ZorB polypeptide, or a ZorD polypeptide, thereby editing the nucleic acid sequence.

In some embodiments, a method of editing a nucleic acid sequence within a cell comprises expressing a component of a Defense System II within said cell, said component comprising a ThsA polypeptide or a ThsB polypeptide, thereby editing the nucleic acid sequence.

In some embodiments, a method of editing a nucleic acid sequence within a cell comprises expressing a component of a Defense System IIIa within said cell, said component comprising a DruA polypeptide, a DruB polypeptide, a DruC polypeptide, a DruD polypeptide, or a DruE polypeptide, thereby editing the nucleic acid sequence.

In some embodiments, a method of editing a nucleic acid sequence within a cell comprises expressing a component of a Defense System IIIb within said cell, said component comprising a DruM polypeptide, a DruF polypeptide, a DruG polypeptide, or a DruE polypeptide, thereby editing the nucleic acid sequence.

In some embodiments, a method of editing a nucleic acid sequence within a cell comprises expressing a component of a Defense System IIIc within said cell, said component comprising a DruH polypeptide, or a DruE polypeptide, thereby editing the nucleic acid sequence.

In some embodiments, a method of editing a nucleic acid sequence within a cell comprises expressing a component of a Defense System IV within said cell, said component comprising a HamA polypeptide or a HamB polypeptide, thereby editing the nucleic acid sequence.

In some embodiments, a method of editing a nucleic acid sequence within a cell comprises expressing a component of a Defense System V within said cell, said component comprising a SduA polypeptide, thereby editing the nucleic acid sequence.

In some embodiments, a method of editing a nucleic acid sequence within a cell comprises expressing a component of a Defense System VI within said cell, said component comprising a GajA polypeptide or a GajB polypeptide, thereby editing the nucleic acid sequence.

In some embodiments, a method of editing a nucleic acid sequence within a cell comprises expressing a component of a Defense System VII within said cell, said component comprising a PtuA polypeptide or a PtuB polypeptide, thereby editing the nucleic acid sequence.

In some embodiments, a method of editing a nucleic acid sequence within a cell comprises expressing a component of a Defense System VIII within said cell, said component comprising a LmuA polypeptide or a LmuB polypeptide, thereby editing the nucleic acid sequence.

In some embodiments, a method of editing a nucleic acid sequence within a cell comprises expressing a component of a Defense System IX within said cell, said component comprising a KwaA polypeptide or a KwaB polypeptide, thereby editing the nucleic acid sequence.

In some embodiments, a method of editing a nucleic acid sequence within a cell comprises expressing a component of a Defense System Xa within said cell, said component comprising a JetA polypeptide, a JetB polypeptide, a JetC polypeptide, or a JetD polypeptide, thereby editing the nucleic acid sequence.

In some embodiments, a method of editing a nucleic acid sequence within a cell comprises expressing a component of a Defense System Xb within said cell, said component comprising a JetAⁱⁱ polypeptide, a JetBⁱⁱ polypeptide, a JetCⁱⁱ polypeptide, or a JetDⁱⁱ polypeptide, thereby editing the nucleic acid sequence.

In some embodiments, a method of editing a nucleic acid sequence within a cell comprises expressing a component of a Defense System Xc within said cell, said component comprising a JetAⁱⁱⁱ polypeptide, a JetBⁱⁱⁱ polypeptide, a JetCⁱⁱⁱ polypeptide, or a JetDⁱⁱ polypeptide, thereby editing the nucleic acid sequence.

In some embodiments, a method of editing a nucleic acid sequence within a cell comprises expressing a gene encoding a component of a Defense System Ia within said cell, said gene comprising a zorA gene, a zorB gene, a zorC gene, or a zorD gene, thereby editing the nucleic acid sequence.

In some embodiments, a method of editing a nucleic acid sequence within a cell comprises expressing a gene encoding a component of a Defense System Ib within said cell, said gene comprising a zorA gene, a zorB gene, or a zorD gene, thereby editing the nucleic acid sequence.

In some embodiments, a method of editing a nucleic acid sequence within a cell comprises expressing a gene encoding a component of a Defense System II within said cell, said gene comprising a thsA gene or a thsB gene, thereby editing the nucleic acid sequence.

In some embodiments, a method of editing a nucleic acid sequence within a cell comprises expressing a gene encoding a component of a Defense System IIIa within said cell, said gene comprising a druA gene, a druB gene, a druC gene, a druD polypeptide, or a druE gene, thereby editing the nucleic acid sequence.

In some embodiments, a method of editing a nucleic acid sequence within a cell comprises expressing a gene encoding a component of a Defense System IIIb within said cell, said gene comprising a druM gene, a druF gene, a druG gene, or a druE gene, thereby editing the nucleic acid sequence.

In some embodiments, a method of editing a nucleic acid sequence within a cell comprises expressing a gene encoding a component of a Defense System IIIc within said cell, said gene comprising a druH gene, or a druE gene, thereby editing the nucleic acid sequence.

In some embodiments, a method of editing a nucleic acid sequence within a cell comprises expressing a gene encoding a component of a Defense System IV within said cell, said gene comprising a hamA gene or a hamB gene, thereby editing the nucleic acid sequence.

In some embodiments, a method of editing a nucleic acid sequence within a cell comprises expressing a gene encoding a component of a Defense System V within said cell, said gene comprising a sduA gene, thereby editing the nucleic acid sequence.

In some embodiments, a method of editing a nucleic acid sequence within a cell comprises expressing a gene encoding a component of a Defense System VI within said cell, said gene comprising a gajA gene or a gajB gene, thereby editing the nucleic acid sequence.

In some embodiments, a method of editing a nucleic acid sequence within a cell comprises expressing a gene encoding a component of a Defense System VII within said cell, said gene comprising a ptuA gene or a ptuB gene, thereby editing the nucleic acid sequence.

In some embodiments, a method of editing a nucleic acid sequence within a cell comprises expressing a gene encoding a component of a Defense System VIII within said cell, said gene comprising a lmuA gene or a lmuB gene, thereby editing the nucleic acid sequence.

In some embodiments, a method of editing a nucleic acid sequence within a cell comprises expressing a gene encoding a component of a Defense System IX within said cell, said gene comprising a kwaA gene or a kwaB gene, thereby editing the nucleic acid sequence.

In some embodiments, a method of editing a nucleic acid sequence within a cell comprises expressing a gene encoding a component of a Defense System Xa within said cell, said gene comprising a jetA gene, a jetB gene, a jetC gene, or a jetD gene, thereby editing the nucleic acid sequence.

In some embodiments, a method of editing a nucleic acid sequence within a cell comprises expressing a gene encoding a component of a Defense System Xb within said cell, said gene comprising a jetAⁱⁱ gene, a jetBⁱⁱ gene, a jetCⁱⁱ gene, or a jetDⁱⁱ gene, thereby editing the nucleic acid sequence.

In some embodiments, a method of editing a nucleic acid sequence within a cell comprises expressing a gene encoding a component of a Defense System Xc within said cell, said gene comprising a jetAⁱⁱⁱ gene, a jetBⁱⁱⁱ gene, a jetCⁱⁱⁱ gene, or a jetDⁱⁱⁱ gene, thereby editing the nucleic acid sequence.

In some embodiments, the nucleic acid sequence being editing is not comprised in a cell expressing the defense system component. In some embodiments, the nucleic acid sequence being editing is not comprised in a cell expression a defense system polypeptide component comprising a ZorA, ZorB, ZorC, ZorD, ZorE, ThsA, ThsB, DruA, DruB, DruC, DruD, DruE, DruM, DruF, DruG, DruH, HamA, HamB, SduA, GajA, GajB, PtuA, PtuB, LmuA, LmuB, KwaA, KwaB, JetA, JetB, JetC, JetD, JetAⁱⁱ, JetBⁱⁱ, JetCⁱⁱ, JetDⁱⁱ, JetAiⁱⁱ, JetBⁱⁱⁱ, JetCⁱⁱⁱ, or JetDⁱⁱⁱ polypeptide.

In some embodiments, the nucleic acid sequence being editing is not comprised in a cell expressing a defense system gene encoding the polypeptide component, said gene comprising a zorA, zorB, zorC, zorD, zorE, thsA, thsB, druA, druB, druC, druD, druE, druM, druF, druG, druH, hamA, hamB, sduA, gajA, gajB, ptuA, ptuB, lmuA, lmuB, kwaA, kwaB, jetA, jetB, jetC, jetD, jetAⁱⁱ, jetBⁱⁱ, jetCⁱⁱ, jetDⁱⁱ, jetAⁱⁱⁱ, jetBⁱⁱⁱ, jetCⁱⁱⁱ, or jetDⁱⁱⁱ gene.

In some embodiments, the nucleic acid sequence being editing is not comprised in a cell expressing a functional defense system component. In some embodiments, the nucleic acid sequence being editing is not comprised in a cell expression a functional defense system polypeptide component comprising a ZorA, ZorB, ZorC, ZorD, ZorE, ThsA, ThsB, DruA, DruB, DruC, DruD, DruE, DruM, DruF, DruG, DruH, HamA, HamB, SduA, GajA, GajB, PtuA, PtuB, LmuA, LmuB, KwaA, KwaB, JetA, JetB, JetC, JetD, JetAⁱⁱ, JetBⁱⁱ, JetCⁱⁱ, JetDⁱⁱ, JetAiⁱⁱ, JetBⁱⁱⁱ, JetCⁱⁱⁱ, or JetDⁱⁱⁱ.

In some embodiments, the nucleic acid sequence being editing is not comprised in a cell expressing a functional defense system gene encoding the polypeptide component, said gene comprising a zorA, zorB, zorC, zorD, zorE, thsA, thsB, druA, druB, druC, druD, druE, druM, druF, druG, druH, hamA, hamB, sduA, gajA, gajB, ptuA, ptuB, lmuA, lmuB, kwaA, kwaB, jetA, jetB, jetC, jetD, jetAⁱⁱ, jetBⁱⁱ, jetCⁱⁱ, jetDⁱⁱ, jetAⁱⁱⁱ, jetBⁱⁱⁱ, jetCⁱⁱⁱ, or jetDⁱⁱⁱ gene.

In some embodiments, there is provided a method of cloning an expression product of interest, the method comprising:

(a) introducing into a nucleic acid construct a polynucleotide encoding the expression product of interest and a defense system component comprising a nucleic acid cleavage activity, or an unwinding activity; and

(b) further introducing into said nucleic acid construct an additional at least one component of the defense system, thereby cloning the expression product of interest.

In some embodiments, step (b) comprises introducing into said nucleic acid construct any other component of the defense system. In some embodiments, step (b) comprises introducing into said nucleic acid construct all other component of the defense system. In some embodiments, step (b) comprises introducing into said nucleic acid construct component of the defense system to form a functional defense system. According to some embodiments the cell does not express an endogenous defense system. In some embodiments, the nucleic acid sequence being edited is not comprised in a cell expressing an endogenous functional version of the defense system.

In a non-limited example, in some embodiments, there is provided a method of cloning an expression product of interest, the method comprising:

(a) introducing into a nucleic acid construct a polynucleotide encoding the expression product of interest and a DruM; and

(b) introducing into said nucleic acid construct a Druantia (Type II) system component selected from the group consisting of: a DruF polypeptide; a DruG polypeptide; and a DruE polypeptide; or any combination thereof, thereby cloning the expression product of interest.

According to some embodiments the cell does not express an endogenous Druantia system. In some embodiments, the nucleic acid sequence being edited is not comprised in a cell expressing an endogenous functional Druantia (Type II) system.

As used herein the term “about” refers to ±10%

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments are disclosed that may be presented in a range format. It should be understood that the description in range format is merely for convenience and brevity and should not be construed as an inflexible limitation on the scope. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

### EXAMPLES

Reference is now made to the following examples, which together with the above descriptions illustrate some embodiments in a non-limiting fashion.

Generally, the nomenclature used herein, and the laboratory procedures utilized, include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are well known in the art and are provided for the convenience of the reader. All the information contained therein is incorporated herein by reference.

### Materials and Methods

Computational Prediction of Defense Systems

A Set of Gene Families Known to Participate in Defense:

A set of pfams and COGs that are known to participate in anti-phage defense was compiled based on the gene families present in Table S10 from Makarova et al. (2011) Defense islands in bacterial and archaeal genomes and prediction of novel defense systems. J Bacteriol. 193: 6039-56, with the addition of pfams/COGs present in the BREX (Goldfarb T, et al. (2015) BREX is a novel phage resistance system widespread in microbial genomes. EMBO J. EMBO Press; 34: 169-83) and DISARM (Ofir G, et al. (2017) DISARM is a widespread bacterial defense system with broad anti-phage activities. Nat Microbiology 2017 doi: 10.1038/s41564-017-0051-0). This set is found in Table 1.

TABLE 1
Known Defense-associated gene families

| | Used in | | | | | |
|---|---|---|---|---|---|---|
| | prediction | | Defense | | Defense | Variability |
| Family | cycle # | Source | system type | Annotation/Comments | score | score |
| pfam12183 | 1 | Makarova et | RM | Restriction endonuclease NotI | 95.9% | 0.38 |
| | | al. (2011)¹ | | | | |
| pfam12161 | 1 | Makarova et | RM | HsdM N-terminal domain | 93.4% | 0.21 |
| | | al. (2011) | | | | |
| pfam12008 | 1 | Makarova et | RM | Type I restriction and | 85.7% | 0.19 |
| | | al. (2011) | | modification enzyme-subunit | | |
| | | | | R C terminal | | |
| pfam11663 | 1 | Makarova et | TA | Toxin with endonuclease | 75.5% | 0.14 |
| | | al. (2011) | | activity YhaV | | |
| pfam11487 | 1 | Makarova et | RM | Type II restriction enzyme | pfam < 20 | pfam < 20 |
| | | al. (2011) | | SfiI | members | members |
| pfam11455 | 1 | Makarova et | new_system | Protein of unknown function | 97.1% | 0.10 |
| | | al. (2011) | | (DUF3018); probable | | |
| | | | | antitoxin associated with | | |
| | | | | Toxin COG2337 | | |
| pfam11198 | 1 | Makarova et | new_system | Protein of unknown function | 80.0% | 0.14 |
| | | al. (2011) | | (DUF2857), associated with | | |
| | | | | COG1475, Spo0J | | |
| pfam11171 | 1 | Makarova et | new_RM | Protein of unknown function | 79.5% | 0.34 |
| | | al. (2011) | | (DUF2958), fused to | | |
| | | | | Methylase, helicase | | |
| pfam10832 | 1 | Makarova et | TA | Protein of unknown function | 96.8% | 0.03 |
| | | al. (2011) | | (DUF2559), TA system | | |
| | | | | component YhfG | | |
| pfam10784 | 1 | Makarova et | TA | Plasmid stability protein | 55.1% | 0.20 |
| | | al. (2011) | | | | |
| pfam10593 | 1 | Makarova et | RM | Z1 domain | 95.3% | 0.42 |
| | | al. (2011) | | | | |
| pfam10592 | 1 | Makarova et | Abi | AIPR protein; abortive | 72.4% | 0.39 |
| | | al. (2011) | | infection phage resistance | | |
| | | | | protein often found in | | |
| | | | | restriction modification | | |
| | | | | system operons; PMID: | | |
| | | | | 18346280 | | |
| pfam10544 | 1 | Makarova et | RM | Type III restriction enzyme, | 55.1% | 0.35 |
| | | al. (2011) | | res subunit | | |
| pfam10543 | 1 | Makarova et | new_system | ORF6N domain | 55.9% | 0.26 |
| | | al. (2011) | | | | |
| pfam10387 | 1 | Makarova et | new_TA | Xre family, TA; Protein of | 59.3% | 0.42 |
| | | al. (2011) | | unknown function (DUF2442) | | |
| pfam10047 | 1 | Makarova et | TA | Protein of unknown function | 86.9% | 0.53 |
| | | al. (2011) | | (DUF2281), same as | | |
| | | | | COG2442 (wHTH) | | |
| pfam10040 | 1 | Makarova et | CRISPR | RAMP, CRISPR related; | 94.6% | 0.34 |
| | | al. (2011) | | Uncharacterized conserved | | |
| | | | | protein (DUF2276) | | |
| pfam09957 | 1 | Makarova et | TA | RHH; Uncharacterized | 96.9% | 0.06 |
| | | al. (2011) | | protein conserved in bacteria | | |
| | | | | (DUF2191); Bacterial | | |
| | | | | antitoxin of type II TA | | |
| | | | | system, VapB | | |
| pfam09827 | 1 | Makarova et | CRISPR | CRISPR associated protein | 93.0% | 0.31 |
| | | al. (2011) | | Cas2 | | |
| pfam09823 | 1 | Makarova et | new_system | Domain of unknown function | 69.1% | 0.52 |
| | | al. (2011) | | (DUF2357) (often fused to | | |
| | | | | recB-like nuclease domain GI: | | |
| | | | | 218891088) | | |
| pfam09720 | 1 | Makarova et | TA | Putative addiction module | 81.8% | 0.44 |
| | | al. (2011) | | component | | |
| pfam09709 | 1 | Makarova et | CRISPR | CRISPR-associated protein | 97.4% | 0.27 |
| | | al. (2011) | | (Cas_Csd1) | | |
| pfam09707 | 1 | Makarova et | CRISPR | CRISPR-associated protein | 98.3% | 0.16 |
| | | al. (2011) | | (Cas_Cas2CT1978) | | |
| pfam09706 | 1 | Makarova et | CRISPR | CRISPR-associated protein | 99.4% | 0.33 |
| | | al. (2011) | | (Cas_CXXC_CXXC) | | |
| pfam09704 | 1 | Makarova et | CRISPR | CRISPR-associated protein | 99.2% | 0.25 |
| | | al. (2011) | | (Cas_Cas5) | | |
| pfam09703 | 1 | Makarova et | CRISPR | CRISPR-associated protein | 100.0% | 0.58 |
| | | al. (2011) | | (Cas_Csa4) | | |
| pfam09702 | 1 | Makarova et | CRISPR | CRISPR-associated protein | 71.1% | 0.72 |
| | | al. (2011) | | (Cas_Csa5) | | |
| pfam09701 | 1 | Makarova et | CRISPR | CRISPR-associated protein | 99.5% | 0.69 |
| | | al. (2011) | | (Cas_Cmr5) | | |
| pfam09700 | 1 | Makarova et | CRISPR | CRISPR-associated protein | 98.2% | 0.65 |
| | | al. (2011) | | (Cas_Cmr3) | | |
| pfam09670 | 1 | Makarova et | CRISPR | CRISPR-associated protein | 89.8% | 0.76 |
| | | al. (2011) | | (Cas_Cas02710) | | |
| pfam09665 | 1 | Makarova et | RM | Type II restriction | 97.4% | 0.18 |
| | | al. (2011) | | endonuclease | | |
| | | | | (RE_Alw26IDE) | | |
| pfam09659 | 1 | Makarova et | CRISPR | CRISPR-associated protein | 78.8% | 0.13 |
| | | al. (2011) | | (Cas_Csm6) | | |
| pfam09658 | 1 | Makarova et | CRISPR | CRISPR-associated protein | pfam < 20 | pfam < 20 |
| | | al. (2011) | | (Cas_Csx9) | members | members |
| pfam09657 | 1 | Makarova et | CRISPR | CRISPR-associated protein | 99.2% | 0.33 |
| | | al. (2011) | | Csx8 (Cas_Csx8) | | |
| pfam09652 | 1 | Makarova et | CRISPR | Putative CRISPR-associated | 94.5% | 0.86 |
| | | al. (2011) | | protein (Cas_VVA1548) | | |
| pfam09651 | 1 | Makarova et | CRISPR | CRISPR-associated protein | 93.0% | 0.76 |
| | | al. (2011) | | (Cas_APE2256) | | |
| pfam09623 | 1 | Makarova et | CRISPR | CRISPR-associated protein | 89.2% | 0.79 |
| | | al. (2011) | | NE0113 (Cas_NE0113) | | |
| pfam09620 | 1 | Makarova et | CRISPR | CRISPR-associated protein | 91.4% | 0.82 |
| | | al. (2011) | | (Cas_c5x3) | | |
| pfam09618 | 1 | Makarova et | CRISPR | CRISPR-associated protein | 98.3% | 0.21 |
| | | al. (2011) | | (Cas_Csy4) | | |
| pfam09617 | 1 | Makarova et | CRISPR | CRISPR-associated protein | 95.9% | 0.47 |
| | | al. (2011) | | GSU0053 (Cas_GSU0053) | | |
| pfam09615 | 1 | Makarova et | CRISPR | CRISPR-associated protein | 99.7% | 0.21 |
| | | al. (2011) | | (Cas_Csy3) | | |
| pfam09614 | 1 | Makarova et | CRISPR | CRISPR-associated protein | 99.8% | 0.19 |
| | | al. (2011) | | (Cas_Csy2) | | |
| pfam09611 | 1 | Makarova et | CRISPR | CRISPR-associated protein | 98.6% | 0.17 |
| | | al. (2011) | | (Cas_Csy1) | | |
| pfam09609 | 1 | Makarova et | CRISPR | CRISPR-associated protein, | 95.0% | 0.47 |
| | | al. (2011) | | GSU0054 family | | |
| | | | | (Cas_GSU0054) | | |
| pfam09573 | 1 | Makarova et | RM | TaqI restriction endonuclease | 85.5% | 0.51 |
| | | al. (2011) | | | | |
| pfam09572 | 1 | Makarova et | RM | XamI restriction endonuclease | 92.3% | 0.36 |
| | | al. (2011) | | | | |
| pfam09571 | 1 | Makarova et | RM | XcyI restriction endonuclease | 91.0% | 0.51 |
| | | al. (2011) | | | | |
| pfam09569 | 1 | Makarova et | RM | ScaI restriction endonuclease | 97.7% | 0.33 |
| | | al. (2011) | | | | |
| pfam09568 | 1 | Makarova et | RM | MjaI restriction endonuclease | 85.0% | 0.34 |
| | | al. (2011) | | | | |
| pfam09565 | 1 | Makarova et | RM | NgoFVII restriction | 95.1% | 0.42 |
| | | al. (2011) | | endonuclease | | |
| pfam09563 | 1 | Makarova et | RM | LlaJI restriction endonuclease | 94.0% | 0.45 |
| | | al. (2011) | | | | |
| pfam09559 | 1 | Makarova et | CRISPR | Cas6 Crispr | 80.0% | 0.40 |
| | | al. (2011) | | | | |
| pfam09556 | 1 | Makarova et | RM | HaeIII restriction | 97.1% | 0.34 |
| | | al. (2011) | | endonuclease | | |
| pfam09549 | 1 | Makarova et | RM | Bpu10I restriction | 96.1% | 0.50 |
| | | al. (2011) | | endonuclease | | |
| pfam09530 | 1 | Makarova et | CRISPR | CRISPR-associated protein | pfam < 20 | pfam < 20 |
| | | al. (2011) | | (cas_TM1812) | members | members |
| pfam09520 | 1 | Makarova et | RM | MjaII restriction endonuclease | 89.7% | 0.41 |
| | | al. (2011) | | | | |
| pfam09517 | 1 | Makarova et | RM | Eco29kI restriction | 95.5% | 0.32 |
| | | al. (2011) | | endonuclease | | |
| pfam09516 | 1 | Makarova et | RM | CfrBI restriction | 95.5% | 0.16 |
| | | al. (2011) | | endonuclease | | |
| pfam09491 | 1 | Makarova et | RM | AlwI restriction endonuclease | 90.5% | 0.28 |
| | | al. (2011) | | | | |
| pfam09485 | 1 | Makarova et | CRISPR | CRISPR-associated protein | 99.6% | 0.16 |
| | | al. (2011) | | Cse2 (CRISPR_cse2) | | |
| pfam09484 | 1 | Makarova et | CRISPR | CRISPR-associated protein | 93.0% | 0.51 |
| | | al. (2011) | | TM1802 (cas_TM1802) | | |
| pfam09481 | 1 | Makarova et | CRISPR | CRISPR-associated protein | 98.4% | 0.17 |
| | | al. (2011) | | Cse1 (CRISPR_cse1) | | |
| pfam09455 | 1 | Makarova et | CRISPR | CRISPR-associated (Cas) | 91.5% | 0.71 |
| | | al. (2011) | | DxTHG family | | |
| pfam09369 | 1 | Makarova et | new_RM | Contains a number of Zn- | 66.2% | 0.26 |
| | | al. (2011) | | binding domains; Domain of | | |
| | | | | unknown function | | |
| | | | | (DUF1998); DISARM | | |
| | | | | system: DrmB | | |
| pfam09344 | 1 | Makarova et | CRISPR | CT1975-like protein | 99.3% | 0.17 |
| | | al. (2011) | | | | |
| pfam09233 | 1 | Makarova et | RM | Restriction endonuclease | 91.6% | 0.39 |
| | | al. (2011) | | EcoRV | | |
| pfam09225 | 1 | Makarova et | RM | Restriction endonuclease | 99.5% | 0.16 |
| | | al. (2011) | | PvuII | | |
| pfam09217 | 1 | Makarova et | RM | Restriction endonuclease | 98.0% | 0.25 |
| | | al. (2011) | | EcoRII, N-terminal | | |
| pfam09208 | 1 | Makarova et | RM | Restriction endonuclease | 96.7% | 0.69 |
| | | al. (2011) | | MspI | | |
| pfam09195 | 1 | Makarova et | RM | Restriction endonuclease | 69.2% | 0.41 |
| | | al. (2011) | | BglII | | |
| pfam09194 | 1 | Makarova et | RM | Restriction endonuclease | 93.8% | 0.50 |
| | | al. (2011) | | BsobI | | |
| pfam09019 | 1 | Makarova et | RM | EcoRII C terminal | 97.7% | 0.17 |
| | | al. (2011) | | | | |
| pfam09002 | 1 | Makarova et | new_system | COG1517 contains RecB | 46.1% | 0.50 |
| | | al. (2011) | | family nuclease and wHTH; | | |
| | | | | Domain of unknown function | | |
| | | | | (DUF1887) | | |
| pfam08972 | 1 | Makarova et | TA | HicA; Domain of unknown | 73.2% | 0.47 |
| | | al. (2011) | | function (DUF1902) | | |
| pfam08849 | 1 | Makarova et | “new system”, | Same as DUF3387; which is | 95.2% | 0.39 |
| | | al. (2011), | BREX | fused to RM (GI: | | |
| | | Goldfarb et | | 209527353); Putative inner | | |
| | | al. (2015) | | membrane protein | | |
| | | | | (DUF1819); pdb: 3BHW; | | |
| | | | | BrxA-Unknown function | | |
| pfam08843 | 1 | Makarova et | new_Abi | Same as COG2253, AbiG; | 70.1% | 0.20 |
| | | al. (2011) | | Domain of unknown function | | |
| | | | | (DUF1814); Nucleotidyl | | |
| | | | | transferase AbiEii toxin, Type | | |
| | | | | IV TA system | | |
| pfam08808 | 1 | Makarova et | TA | RES domain | 66.6% | 0.14 |
| | | al. (2011) | | | | |
| pfam08798 | 1 | Makarova et | CRISPR | CRISPR associated protein | 98.6% | 0.18 |
| | | al. (2011) | | | | |
| pfam08780 | 1 | Makarova et | TA | HEPN; Nucleotidyltransferase | 91.2% | 0.32 |
| | | al. (2011) | | substrate binding protein like | | |
| pfam08747 | 1 | Makarova et | new_RM, | Domain of unknown function | 97.9% | 0.45 |
| | | al. (2011), | BREX | (DUF1788); BrxB-Unknown | | |
| | | Goldfarb et | | function | | |
| | | al. (2015) | | | | |
| pfam08665 | 1 | Makarova et | “new system” | PglZ domain, Alkaline | 69.4% | 0.45 |
| | | al. (2011) | | phosphatase clan. BREX | | |
| | | | | system: PglZ | | |
| pfam08463 | 1 | Makarova et | RM | EcoEI R protein C-terminal | 97.3% | 0.28 |
| | | al. (2011) | | | | |
| pfam07927 | 1 | Makarova et | TA | toxin component, HicA | 89.7% | 0.24 |
| | | al. (2011) | | family; YcfA-like protein; | | |
| | | | | HicA toxin of bacterial toxin- | | |
| | | | | antitoxin | | |
| pfam07805 | 1 | Makarova et | TA | HipA-like N-terminal domain | 57.3% | 0.24 |
| | | al. (2011) | | | | |
| pfam07804 | 1 | Makarova et | TA | HipA-like C-terminal domain | 56.2% | 0.24 |
| | | al. (2011) | | | | |
| pfam07751 | 1 | Makarova et | Abi | Abi-like protein | 66.3% | 0.17 |
| | | al. (2011) | | | | |
| pfam07669 | 1 | Makarova et | RM | Eco57I restriction | 74.2% | 0.29 |
| | | al. (2011) | | endonuclease | | |
| pfam07215 | 1 | Makarova et | new_RM | Protein of unknown function | 88.1% | 0.53 |
| | | al. (2011) | | (DUF1419) linked to | | |
| | | | | methylase COG0827 | | |
| pfam06616 | 1 | Makarova et | RM | BsuBI/PstI restriction | 81.7% | 0.34 |
| | | al. (2011) | | endonuclease C-terminus | | |
| pfam06414 | 1 | Makarova et | TA | Zeta toxin | 35.1% | 0.27 |
| | | al. (2011) | | | | |
| pfam06296 | 1 | Makarova et | TA | Toxin component, RelE | 90.5% | 0.13 |
| | | al. (2011) | | family Protein of unknown | | |
| | | | | function (DUF1044) | | |
| pfam06114 | 1 | Makarova et | TA | Same as COG2856; Domain | 48.5% | 0.16 |
| | | al. (2011) | | of unknown function | | |
| | | | | (DUF955) | | |
| pfam05942 | 1 | Makarova et | TA | Archaeal PaREP1/PaREP8 | 70.7% | 0.34 |
| | | al. (2011) | | family | | |
| pfam05534 | 1 | Makarova et | TA | HicB family | 76.4% | 0.21 |
| | | al. (2011) | | | | |
| pfam05016 | 1 | Makarova et | TA | Plasmid stabilisation system | 77.4% | 0.18 |
| | | al. (2011) | | protein; ParE toxin of type II | | |
| | | | | toxin-antitoxin system, parDE | | |
| pfam04851 | 1 | Makarova et | RM | Type III restriction enzyme, | 55.5% | 0.16 |
| | | al. (2011) | | res subunit | | |
| pfam04556 | 1 | Makarova et | RM | DpnII restriction | 93.8% | 0.19 |
| | | al. (2011) | | endonuclease | | |
| pfam04555 | 1 | Makarova et | RM | Restriction endonuclease | 90.0% | 0.39 |
| | | al. (2011) | | XhoI | | |
| pfam04149 | 1 | Makarova et | TA | DUF397, see our TA paper | 51.5% | 0.12 |
| | | al. (2011) | | | | |
| pfam04014 | 1 | Makarova et | TA | SpoVT/AbrB like domain; | 77.3% | 0.16 |
| | | al. (2011) | | Antidote-toxin recognition | | |
| | | | | MazE, bacterial antitoxin | | |
| pfam03787 | 1 | Makarova et | CRISPR | RAMP superfamily | 98.4% | 0.44 |
| | | al. (2011) | | | | |
| pfam03681 | 1 | Makarova et | TA | HicB family; Uncharacterized | pfam < 20 | pfam < 20 |
| | | al. (2011) | | protein family (UPF0150) | members | members |
| pfam03230 | 1 | Makarova et | RM | Antirestriction protein | 57.8% | 0.14 |
| | | al. (2011) | | | | |
| pfam02963 | 1 | Makarova et | RM | Restriction endonuclease | 61.6% | 0.40 |
| | | al. (2011) | | EcoRI | | |
| pfam02923 | 1 | Makarova et | RM | Restriction endonuclease | 97.4% | 0.47 |
| | | al. (2011) | | BamHI | | |
| pfam02604 | 1 | Makarova et | TA | Antitoxin Phd_Yet-M, type II | 90.5% | 0.13 |
| | | al. (2011) | | toxin-antitoxin system | | |
| pfam01930 | 1 | Makarova et | CRISPR | Domain of unknown function | 90.5% | 0.37 |
| | | al. (2011) | | DUF83 | | |
| pfam01909 | 1 | Makarova et | TA | Nucleotidyltransferase | 47.6% | 0.26 |
| | | al. (2011) | | domain, COG1669 | | |
| pfam01867 | 1 | Makarova et | CRISPR | CRISPR associated protein | 97.4% | 0.26 |
| | | al. (2011) | | Cas1 | | |
| pfam01850 | 1 | Makarova et | TA | PIN domain | 76.0% | 0.11 |
| | | al. (2011) | | | | |
| pfam01845 | 1 | Makarova et | TA | CcdB protein | 89.4% | 0.15 |
| | | al. (2011) | | | | |
| pfam01420 | 1 | Makarova et | RM | Type I restriction | 91.8% | 0.20 |
| | | al. (2011) | | modification DNA specificity | | |
| | | | | domain | | |
| pfam14390 | 1 | Ofir et al. | DISARM- | Putative PD-(D/E)XK family | 98.9% | 0.42 |
| | | (2017) | associated | member, (DUF4420) | | |
| pfam13659 | 1 | Goldfarb et | BREX, | PglX-Adenine-specific | 63.3% | 0.21 |
| | | al. (2015)², | DISARM | methylase; DrmMI- | | |
| | | Ofir et al. | | Superfamily SSF5335 (S- | | |
| | | (2017)³ | | adenosyl-L-methionine- | | |
| | | | | dependent | | |
| | | | | methyltransferases); | | |
| | | | | Methyltransferase domain | | |
| pfam13589 | 1 | Ofir et al. | DISARM- | Histidine kinase-, DNA | 33.5% | 0.21 |
| | | (2017) | associated | gyrase B-, and HSP90-like | | |
| | | | | ATPase | | |
| pfam13361 | 1 | Ofir et al. | DISARM- | UvrD-like helicase C-terminal | 50.2% | 0.08 |
| | | (2017) | associated | domain | | |
| pfam13182 | 1 | Goldfarb et | BREX | Protein of unknown function | 94.9% | 0.31 |
| | | al. (2015) | | (DUF4007); BrxP- | | |
| | | | | Phosphoadenosine | | |
| | | | | phosphosulfate reductase | | |
| pfam10923 | 1 | Goldfarb et | BREX | BrxC/PglY, BrxD-ATP | 93.1% | 0.28 |
| | | al. (2015) | | binding; P-loop Domain of | | |
| | | | | unknown function (DUF2791) | | |
| pfam04465 | 1 | Goldfarb et | BREX | BrxC/PglY-ATP binding; | 93.1% | 0.50 |
| | | al. (2015) | | Protein of unknown function | | |
| | | | | (DUF499) | | |
| pfam03852 | 1 | Ofir et al. | DISARM- | DNA mismatch endonuclease | 93.0% | 0.18 |
| | | (2017) | associated | Vsr | | |
| pfam01555 | 1 | Goldfarb et | BREX | DNA methylase; PglXI- | 64.4% | 0.20 |
| | | al. (2015) | | Adenine-specific methylase | | |
| pfam01507 | 1 | Goldfarb et | BREX | Phosphoadenosine | 62.9% | 0.07 |
| | | al. (2015) | | phosphosulfate reductase | | |
| | | | | family; BrxP- | | |
| | | | | Phosphoadenosine | | |
| | | | | phosphosulfate reductase | | |
| pfam00580 | 1 | Ofir et al. | DISARM- | UvrD/REP helicase N- | 47.4% | 0.09 |
| | | (2017) | associated | terminal domain | | |
| pfam00271 | 1 | Ofir et al. | DISARM | DrmA; Helicase conserved C- | 39.0% | 0.10 |
| | | (2017) | | terminal domain | | |
| pfam00176 | 1 | Ofir et al. | DISARM | DrmD; SNF2 family N- | 46.8% | 0.23 |
| | | (2017) | | terminal domain | | |
| pfam00145 | 1 | Ofir et al. | DISARM | DrmMII; C-5 cytosine- | 72.5% | 0.19 |
| | | (2017) | | specific DNA methylase | | |
| pfam00004 | 1 | Ofir et al. | DISARM- | ATPase family associated | 30.1% | 0.07 |
| | | (2017) | associated | with various cellular activities | | |
| | | | | (AAA) | | |
| COG5654 | 1 | Makarova et | TA | Uncharacterized conserved | | |
| | | al. (2011) | | protein | | |
| COG5642 | 1 | Makarova et | TA | Uncharacterized conserved | | |
| | | al. (2011) | | protein | | |
| COG4861 | 1 | Makarova et | Abi | Uncharacterized protein | | |
| | | al. (2011) | | conserved in bacteria | | |
| COG5611 | 1 | Makarova et | TA | PIN domain | | |
| | | al. (2011) | | | | |
| COG4849 | 1 | Makarova et | Abi | Uncharacterized protein | | |
| | | al. (2011) | | conserved in bacteria | | |
| COG5606 | 1 | Makarova et | TA | Uncharacterized conserved | | |
| | | al. (2011) | | small protein | | |
| COG4823 | 1 | Makarova et | Abi | Abortive infection | | |
| | | al. (2011) | | bacteriophage resistance | | |
| | | | | protein | | |
| COG5573 | 1 | Makarova et | TA | Predicted nucleic-acid- | | |
| | | al. (2011) | | binding protein, contains PIN | | |
| | | | | domain | | |
| COG4804 | 1 | Makarova et | new_system | Predicted nuclease of | | |
| | | al. (2011) | | restriction endonuclease-like | | |
| | | | | fold | | |
| COG5551 | 1 | Makarova et | CRISPR | CRISPR system related | | |
| | | al. (2011) | | protein, RAMP superfamily | | |
| COG4748 | 1 | Makarova et | RM | Uncharacterized conserved | | |
| | | al. (2011) | | protein | | |
| COG5519 | 1 | Makarova et | new_system | Superfamily II helicase or | | |
| | | al. (2011) | | inactivated derivatives, fused | | |
| | | | | to primase | | |
| COG4737 | 1 | Makarova et | TA | Uncharacterized protein | | |
| | | al. (2011) | | conserved in bacteria | | |
| COG5499 | 1 | Makarova et | TA | Predicted transcription | | |
| | | al. (2011) | | regulator containing HTH | | |
| | | | | domain | | |
| COG4710 | 1 | Makarova et | TA | Predicted DNA-binding | | |
| | | al. (2011) | | protein with an HTH domain | | |
| COG5464 | 1 | Makarova et | new_RM | RecB-family nuclease domain | | |
| | | al. (2011) | | (see TIGR01784), linked to | | |
| | | | | COG1479 | | |
| COG4694 | 1 | Makarova et | Abi | Wobble nucleotide-excising | | |
| | | al. (2011) | | tRNase | | |
| COG5450 | 1 | Makarova et | TA | Transcription regulator of the | | |
| | | al. (2011) | | Arc/MetJ class | | |
| COG4691 | 1 | Makarova et | TA | Plasmid stability protein | | |
| | | al. (2011) | | | | |
| COG5340 | 1 | Makarova et | Abi | Predicted transcriptional | | |
| | | al. (2011) | | regulator | | |
| COG4683 | 1 | Makarova et | TA | Uncharacterized protein | | |
| | | al. (2011) | | conserved in bacteria | | |
| COG5304 | 1 | Makarova et | TA | Uncharacterized protein | | |
| | | al. (2011) | | conserved in bacteria | | |
| COG4680 | 1 | Makarova et | TA | Uncharacterized protein | | |
| | | al. (2011) | | conserved in bacteria | | |
| COG5302 | 1 | Makarova et | TA | Post-segregation antitoxin | | |
| | | al. (2011) | | (ccd killing mechanism | | |
| | | | | protein) encoded by the F | | |
| | | | | plasmid | | |
| COG4679 | 1 | Makarova et | TA | Phage-related protein | | |
| | | al. (2011) | | | | |
| COG4951 | 1 | Makarova et | RM | Uncharacterized protein | | |
| | | al. (2011) | | conserved in bacteria | | |
| COG4646 | 1 | Makarova et | new_system | DNA methylase, see | | |
| | | al. (2011) | | COG0553 | | |
| COG4914 | 1 | Makarova et | Abi | Predicted | | |
| | | al. (2011) | | nucleotidyltransferase | | |
| COG4636 | 1 | Makarova et | TA | Endonuclease, Uma2 family | | |
| | | al. (2011) | | (restriction endonuclease fold) | | |
| COG4889 | 1 | Makarova et | RM | Predicted helicase | | |
| | | al. (2011) | | | | |
| COG4634 | 1 | Makarova et | TA | PIN family nuclease, potential | | |
| | | al. (2011) | | toxin-antitoxin system | | |
| | | | | component | | |
| COG4456 | 1 | Makarova et | TA | Virulence-associated protein | | |
| | | al. (2011) | | or related protein | | |
| COG4453 | 1 | Makarova et | TA | Uncharacterized protein | | |
| | | al. (2011) | | conserved in bacteria | | |
| COG4423 | 1 | Makarova et | TA | Uncharacterized protein | | |
| | | al. (2011) | | conserved in bacteria | | |
| COG4374 | 1 | Makarova et | TA | PIN domain nuclease, a | | |
| | | al. (2011) | | component of toxin-antitoxin | | |
| | | | | system (PIN domain) | | |
| COG4343 | 1 | Makarova et | CRISPR | CRISPR-associated protein, | | |
| | | al. (2011) | | RecB family exonuclease | | |
| COG4268 | 1 | Makarova et | RM | McrBC 5-methylcytosine | | |
| | | al. (2011) | | restriction system component | | |
| COG4227 | 1 | Makarova et | new_system | Antirestriction protein | | |
| | | al. (2011) | | | | |
| COG4226 | 1 | Makarova et | TA | Predicted nuclease of the | | |
| | | al. (2011) | | RNAse H fold, HicB family | | |
| COG4127 | 1 | Makarova et | new_RM | Mrr restriction endonuclease | | |
| | | al. (2011) | | domain | | |
| COG4118 | 1 | Makarova et | TA | Antitoxin (DNA binding | | |
| | | al. (2011) | | domain) of toxin-antitoxin | | |
| | | | | stability system | | |
| COG4115 | 1 | Makarova et | TA | Uncharacterized protein | | |
| | | al. (2011) | | conserved in bacteria | | |
| COG4113 | 1 | Makarova et | TA | Predicted nucleic acid-binding | | |
| | | al. (2011) | | protein, contains PIN domain | | |
| COG4096 | 1 | Makarova et | RM | Type I site-specific | | |
| | | al. (2011) | | restriction-modification | | |
| | | | | system, R (restriction) subunit | | |
| | | | | or related helicase | | |
| COG4006 | 1 | Makarova et | CRISPR | Uncharacterized protein | | |
| | | al. (2011) | | conserved in archaea | | |
| COG3950 | 1 | Makarova et | new_system | Predicted ATP-binding | | |
| | | al. (2011) | | protein involved in virulence | | |
| COG3943 | 1 | Makarova et | new_TA | Virulence protein | | |
| | | al. (2011) | | | | |
| COG3905 | 1 | Makarova et | TA | Predicted transcriptional | | |
| | | al. (2011) | | regulator | | |
| COG3886 | 1 | Makarova et | RM | Predicted HKD family | | |
| | | al. (2011) | | nuclease; type III restriction | | |
| | | | | protein res subunit | | |
| COG3832 | 1 | Makarova et | TA | Uncharacterized conserved | | |
| | | al. (2011) | | protein | | |
| COG3744 | 1 | Makarova et | TA | PIN domain nuclease, a | | |
| | | al. (2011) | | component of toxin-antitoxin | | |
| | | | | system (PIN domain) | | |
| COG3742 | 1 | Makarova et | TA | Uncharacterized protein | | |
| | | al. (2011) | | conserved in bacteria | | |
| COG3692 | 1 | Makarova et | TA | Uncharacterized protein | | |
| | | al. (2011) | | conserved in bacteria | | |
| COG3668 | 1 | Makarova et | TA | Plasmid stabilization system | | |
| | | al. (2011) | | protein | | |
| COG3657 | 1 | Makarova et | TA | Uncharacterized protein | | |
| | | al. (2011) | | conserved in bacteria | | |
| COG3654 | 1 | Makarova et | TA | Prophage maintenance system | | |
| | | al. (2011) | | killer protein | | |
| COG3649 | 1 | Makarova et | CRISPR | CRISPR system related | | |
| | | al. (2011) | | protein | | |
| COG3636 | 1 | Makarova et | TA | Predicted transcriptional | | |
| | | al. (2011) | | regulator | | |
| COG3609 | 1 | Makarova et | TA | Predicted transcriptional | | |
| | | al. (2011) | | regulators containing the | | |
| | | | | CopG/Arc/MetJ DNA-binding | | |
| | | | | domain | | |
| COG3598 | 1 | Makarova et | new_system | RecA-family ATPase, fused | | |
| | | al. (2011) | | to TOPRIM | | |
| COG3587 | 1 | Makarova et | RM | Restriction endonuclease | | |
| | | al. (2011) | | | | |
| COG3586 | 1 | Makarova et | new_system | Some protein fused to | | |
| | | al. (2011) | | domains similar to N terminal | | |
| | | | | regions found in type I | | |
| | | | | restriction enzyme R (HSDR) | | |
| | | | | proteins and RloF (gi: | | |
| | | | | 93006430) | | |
| COG3550 | 1 | Makarova et | TA | Toxin module HipA, protein | | |
| | | al. (2011) | | kinase of phosphatidylinositol | | |
| | | | | 3/4-kinase superfamily | | |
| COG3549 | 1 | Makarova et | TA | Plasmid maintenance system | | |
| | | al. (2011) | | killer protein | | |
| COG3514 | 1 | Makarova et | TA | Uncharacterized protein | | |
| | | al. (2011) | | conserved in bacteria | | |
| COG3513 | 1 | Makarova et | CRISPR | Predicted CRISPR-associated | | |
| | | al. (2011) | | nuclease, contains | | |
| | | | | McrA/HNH-nuclease and | | |
| | | | | RuvC-like nuclease domain | | |
| COG3512 | 1 | Makarova et | CRISPR | CRISPR-associated protein, | | |
| | | al. (2011) | | Cas2 homolog | | |
| COG3472 | 1 | Makarova et | new_system | Uncharacterized; often fused | | |
| | | al. (2011) | | to COG1479 | | |
| COG3440 | 1 | Makarova et | RM | Predicted restriction | | |
| | | al. (2011) | | endonuclease | | |
| COG3410 | 1 | Makarova et | Abi | Uncharacterized conserved | | |
| | | al. (2011) | | protein | | |
| COG3392 | 1 | Makarova et | RM | Adenine-specific DNA | | |
| | | al. (2011) | | methylase | | |
| COG3372 | 1 | Makarova et | new_RM | Predicted nuclease of | | |
| | | al. (2011) | | restriction endonuclease-like | | |
| | | | | fold; associated with | | |
| | | | | COG1061 (RM) | | |
| COG3344 | 1 | Makarova et | Abi | Retron-type reverse | | |
| | | al. (2011) | | transcriptase | | |
| COG3337 | 1 | Makarova et | CRISPR | CRISPR system related | | |
| | | al. (2011) | | protein | | |
| COG3311 | 1 | Makarova et | TA | HTH_MerR_SF | | |
| | | al. (2011) | | | | |
| COG3183 | 1 | Makarova et | RM | Predicted restriction | | |
| | | al. (2011) | | endonuclease | | |
| COG3177 | 1 | Makarova et | TA | Fic family protein | | |
| | | al. (2011) | | | | |
| COG3093 | 1 | Makarova et | TA | Plasmid maintenance system | | |
| | | al. (2011) | | antidote protein | | |
| COG3077 | 1 | Makarova et | TA | DNA-damage-inducible | | |
| | | al. (2011) | | protein J | | |
| COG3041 | 1 | Makarova et | TA | Uncharacterized protein | | |
| | | al. (2011) | | conserved in bacteria | | |
| COG2944 | 1 | Makarova et | TA | Predicted transcriptional | | |
| | | al. (2011) | | regulator | | |
| COG2929 | 1 | Makarova et | TA | Uncharacterized protein | | |
| | | al. (2011) | | conserved in bacteria | | |
| COG2886 | 1 | Makarova et | TA | Uncharacterized small protein | | |
| | | al. (2011) | | | | |
| COG2880 | 1 | Makarova et | TA | Uncharacterized protein | | |
| | | al. (2011) | | conserved in archaea | | |
| COG2865 | 1 | Makarova et | new_system | Predicted transcriptional | | |
| | | al. (2011) | | regulator containing an HTH | | |
| | | | | domain and diverged ATP- | | |
| | | | | binding domain (Schlafen- | | |
| | | | | like) | | |
| COG2856 | 1 | Makarova et | TA | Predicted Zn peptidase | | |
| | | al. (2011) | | | | |
| COG2852 | 1 | Makarova et | RM | Very-short-patch-repair | | |
| | | al. (2011) | | endonuclease | | |
| COG2810 | 1 | Makarova et | RM | Predicted type IV restriction | | |
| | | al. (2011) | | endonuclease | | |
| COG2521 | 1 | Makarova et | new_RM | Predicted archaeal | | |
| | | al. (2011) | | methyltransferase | | |
| COG2445 | 1 | Makarova et | TA | Uncharacterized conserved | | |
| | | al. (2011) | | protein | | |
| COG2442 | 1 | Makarova et | TA | Uncharacterized conserved | | |
| | | al. (2011) | | protein | | |
| COG2405 | 1 | Makarova et | TA | Predicted nucleic acid-binding | | |
| | | al. (2011) | | protein, contains PIN domain | | |
| COG2402 | 1 | Makarova et | TA | Predicted nucleic acid-binding | | |
| | | al. (2011) | | protein, contains PIN domain | | |
| COG2361 | 1 | Makarova et | TA | Uncharacterized conserved | | |
| | | al. (2011) | | protein | | |
| COG2337 | 1 | Makarova et | TA | Toxin-antitoxin addiction | | |
| | | al. (2011) | | module toxin component | | |
| | | | | MazF (an endoRNAse) | | |
| COG2336 | 1 | Makarova et | TA | Toxin-antitoxin addiction | | |
| | | al. (2011) | | module antitoxin component | | |
| | | | | MazE | | |
| COG2254 | 1 | Makarova et | CRISPR | Predicted HD superfamily | | |
| | | al. (2011) | | hydrolase, possibly a nuclease | | |
| COG2253 | 1 | Makarova et | Abi | Uncharacterized conserved | | |
| | | al. (2011) | | protein | | |
| COG2250 | 1 | Makarova et | TA | HEPN domain | | |
| | | al. (2011) | | | | |
| COG2189 | 1 | Makarova et | RM | Adenine specific DNA | | |
| | | al. (2011) | | methylase Mod | | |
| COG2184 | 1 | Makarova et | TA | Protein involved in cell | | |
| | | al. (2011) | | division | | |
| COG2161 | 1 | Makarova et | TA | Antitoxin (DNA-binding | | |
| | | al. (2011) | | domain) of toxin-antitoxin | | |
| | | | | stability system | | |
| COG2026 | 1 | Makarova et | TA | Cytotoxic translational | | |
| | | al. (2011) | | repressor of toxin-antitoxin | | |
| | | | | stability system | | |
| COG2002 | 1 | Makarova et | TA | Transcriptional regulator | | |
| | | al. (2011) | | AbrB | | |
| COG1895 | 1 | Makarova et | TA | Uncharacterized conserved | | |
| | | al. (2011) | | protein related to C-terminal | | |
| | | | | domain of eukaryotic | | |
| | | | | chaperone, SACSIN | | |
| COG1857 | 1 | Makarova et | CRISPR | CRISPR system related | | |
| | | al. (2011) | | protein | | |
| COG1848 | 1 | Makarova et | TA | Predicted nucleic acid-binding | | |
| | | al. (2011) | | protein, contains PIN domain | | |
| COG1787 | 1 | Makarova et | RM | Endonuclease, HJR/Mrr/RecB | | |
| | | al. (2011) | | family | | |
| COG1769 | 1 | Makarova et | CRISPR | CRISPR system related | | |
| | | al. (2011) | | protein, RAMP superfamily | | |
| COG1753 | 1 | Makarova et | TA | Predicted antitoxin, copG | | |
| | | al. (2011) | | family | | |
| COG1743 | 1 | Makarova et | RM, BREX | Adenine-specific DNA | | |
| | | al. (2011), | | methylase containing a Zn- | | |
| | | Goldfarb et | | ribbon; PglXI-Adenine- | | |
| | | al. (2015) | | specific methylase | | |
| COG1724 | 1 | Makarova et | TA | Predicted RNA binding | | |
| | | al. (2011) | | protein (dsRBD-like fold), | | |
| | | | | HicA family | | |
| COG1715 | 1 | Makarova et | RM | Restriction endonuclease | | |
| | | al. (2011) | | | | |
| COG1708 | 1 | Makarova et | TA | Predicted | | |
| | | al. (2011) | | nucleotidyltransferase | | |
| COG1700 | 1 | Makarova et | new_RM | Contains PD_(D/E)xK | | |
| | | al. (2011) | | nuclease odmain, DUF524 | | |
| COG1688 | 1 | Makarova et | CRISPR | CRISPR system related | | |
| | | al. (2011) | | protein, RAMP superfamily | | |
| COG1669 | 1 | Makarova et | TA | Nucleotidyltransferase | | |
| | | al. (2011) | | | | |
| COG1604 | 1 | Makarova et | CRISPR | CRISPR system related | | |
| | | al. (2011) | | protein, RAMP superfamily | | |
| COG1598 | 1 | Makarova et | TA | Predicted nuclease of the | | |
| | | al. (2011) | | RNAse H fold, HicB family | | |
| COG1583 | 1 | Makarova et | CRISPR | CRISPR system related | | |
| | | al. (2011) | | protein, RAMP superfamily | | |
| COG1569 | 1 | Makarova et | TA | Predicted nucleic acid-binding | | |
| | | al. (2011) | | protein, contains PIN domain | | |
| COG1567 | 1 | Makarova et | CRISPR | CRISPR system related | | |
| | | al. (2011) | | protein, RAMP superfamily | | |
| COG1518 | 1 | Makarova et | CRISPR | CRISPR-associated protein | | |
| | | al. (2011) | | Cas1 | | |
| COG1517 | 1 | Makarova et | CRISPR | CRISPR system related | | |
| | | al. (2011) | | protein | | |
| COG1487 | 1 | Makarova et | TA | Predicted nucleic acid-binding | | |
| | | al. (2011) | | protein, contains PIN domain | | |
| COG1479 | 1 | Makarova et | new_system | RloF-like, possible RM | | |
| | | al. (2011) | | component (contains HNH- | | |
| | | | | type nuclease domain) | | |
| COG1476 | 1 | Makarova et | TA | Predicted transcriptional | | |
| | | al. (2011) | | regulator, xre family | | |
| COG1475 | 1 | Makarova et | new_system | Stage 0 sporulation protein J | | |
| | | al. (2011) | | (antagonist of Soj); contains | | |
| | | | | ParB-like nuclease domain | | |
| COG1468 | 1 | Makarova et | CRISPR | CRISPR-associated protein | | |
| | | al. (2011) | | Cas4 (RecB family | | |
| | | | | exonuclease) | | |
| COG1431 | 1 | Makarova et | pAgo | Argonaute homolog, | | |
| | | al. (2011) | | implicated in RNA | | |
| | | | | metabolism | | |
| COG1421 | 1 | Makarova et | CRISPR | CRISPR system related | | |
| | | al. (2011) | | protein | | |
| COG1403 | 1 | Makarova et | RM | Restriction endonuclease, | | |
| | | al. (2011) | | McrA/HNH family | | |
| COG1401 | 1 | Makarova et | RM | GTPase subunit of restriction | | |
| | | al. (2011) | | endonuclease | | |
| COG1396 | 1 | Makarova et | TA | Predicted transcriptional | | |
| | | al. (2011) | | regulator, xre family | | |
| COG1367 | 1 | Makarova et | CRISPR | CRISPR system related | | |
| | | al. (2011) | | protein, RAMP superfamily | | |
| COG1353 | 1 | Makarova et | CRISPR | Predicted CRISPR-associated | | |
| | | al. (2011) | | polymerase | | |
| COG1343 | 1 | Makarova et | CRISPR | CRISPR-associated protein | | |
| | | al. (2011) | | Cas2 | | |
| COG1337 | 1 | Makarova et | CRISPR | CRISPR system related | | |
| | | al. (2011) | | protein, RAMP superfamily | | |
| COG1336 | 1 | Makarova et | CRISPR | CRISPR system related | | |
| | | al. (2011) | | protein, RAMP superfamily | | |
| COG1332 | 1 | Makarova et | CRISPR | CRISPR system related | | |
| | | al. (2011) | | protein, RAMP superfamily | | |
| COG1203 | 1 | Makarova et | CRISPR | CRISPR-associated helicase | | |
| | | al. (2011) | | Cas3 | | |
| COG1112 | 1 | Makarova et | new_system | Superfamily I DNA/RNA | | |
| | | al. (2011) | | helicase | | |
| COG1106 | 1 | Makarova et | Abi | Predicted ATPase | | |
| | | al. (2011) | | | | |
| COG1061 | 1 | Makarova et | RM | DNA or RNA helicase of | | |
| | | al. (2011) | | superfamily II | | |
| COG1002 | 1 | Makarova et | RM, BREX | Type II restriction enzyme, | | |
| | | al. (2011), | | methylase subunit; PglX- | | |
| | | Goldfarb et | | Adenine-specific methylase | | |
| | | al. (2015) | | | | |
| COG0864 | 1 | Makarova et | TA | Transcriptional regulator, | | |
| | | al. (2011) | | CopG/Arc/MetJ family | | |
| | | | | (DNA-binding and a metal- | | |
| | | | | binding domains) | | |
| COG0863 | 1 | Makarova et | RM, BREX | DNA modification methylase; | | |
| | | al. (2011), | | PglXI-Adenine-specific | | |
| | | Goldfarb et | | methylase | | |
| | | al. (2015) | | | | |
| COG0827 | 1 | Makarova et | RM | Adenine-specific DNA | | |
| | | al. (2011) | | methylase | | |
| COG0732 | 1 | Makarova et | RM | Restriction endonuclease S | | |
| | | al. (2011) | | subunit | | |
| COG0640 | 1 | Makarova et | TA | Transcriptional regulator | | |
| | | al. (2011) | | containing HTH domain, | | |
| | | | | ArsR family | | |
| COG0610 | 1 | Makarova et | RM | Type I site-specific | | |
| | | al. (2011) | | restriction-modification | | |
| | | | | system, R (restriction) subunit | | |
| | | | | or related helicase | | |
| COG0338 | 1 | Makarova et | RM | Site-specific DNA methylase | | |
| | | al. (2011) | | | | |
| COG0286 | 1 | Makarova et | RM, BREX | Type I restriction- | | |
| | | al. (2011), | | modification system | | |
| | | Goldfarb et | | methyltransferase subunit; | | |
| | | al. (2015) | | PglX-Adenine-specific | | |
| | | | | methylase | | |
| COG0270 | 1 | Makarova et | RM, | Dcm, Site-specific DNA- | | |
| | | al. (2011), | DISARM | cytosine methylase; DrmMII | | |
| | | Ofir et al. | | | | |
| | | (2017) | | | | |
| COG4930 | 1 | Goldfarb et | BREX | BrxL-Lon-like protease | | |
| | | al. (2015) | | | | |
| COG3655 | 1 | Ofir et al. | DISARM- | DNA-binding transcriptional | | |
| | | (2017) | associated | regulator, XRE family | | |
| COG1404 | 1 | Ofir et al. | DISARM- | Serine protease, subtilisin | | |
| | | (2017) | associated | family | | |
| COG1205 | 1 | Ofir et al. | DISARM- | ATP-dependent helicase | | |
| | | (2017) | associated | YprA, contains C-terminal | | |
| | | | | metal-binding DUF1998 | | |
| | | | | domain | | |
| COG1201 | 1 | Goldfarb et | BREX | BrxHI-Lhr-like helicase | | |
| | | al. (2015) | | | | |
| COG0553 | 1 | Goldfarb et | BREX, | BrxHII-DNA/RNA | | |
| | | al. (2015), | DISARM | helicases; DrmD; HepA, | | |
| | | Ofir et al. | | Superfamily II DNA or RNA | | |
| | | (2017) | | helicase, SNF2 family | | |
| COG0515 | 1 | Goldfarb et | BREX | PglW-Serine/threonine | | |
| | | al. (2015) | | protein kinase | | |
| COG0210 | 1 | Ofir et al. | DISARM- | UvrD, Superfamily I DNA or | | |
| | | (2017) | associated | RNA helicase | | |
| COG0175 | 1 | Goldfarb et | BREX | BrxP-Phosphoadenosine | | |
| | | al. (2015) | | phosphosulfate reductase | | |
| pfam13091 | 2 | Ofir et al. | DISARM | DrmC; PLD-like domain | 44.2% | 0.14 |
| | | (2017) | | | | |
| pfam01881 | 2 | known | CRISPR | CRISPR associated protein | 92.3% | 0.44 |
| | | system | | Cas6 | | |
| | | found in | | | | |
| | | round 1 | | | | |
| pfam01905 | 2 | known | CRISPR | CRISPR-associated negative | 99.3% | 0.41 |
| | | system | | auto-regulator DevR/Csa2 | | |
| | | found in | | | | |
| | | round 1 | | | | |
| pfam01954 | 2 | known | TA | Protein of unknown function | 89.8% | 0.59 |
| | | system | | DUF104. Probably a new | | |
| | | found in | | antitoxin; the associated gene | | |
| | | round 1 | | is a PIN-domain protein, | | |
| | | | | known toxin | | |
| pfam02697 | 2 | known | TA | Uncharacterized ACR, | 89.6% | 0.42 |
| | | system | | COG1753 | | |
| | | found in | | | | |
| | | round 1 | | | | |
| pfam02980 | 2 | known | RM | Restriction endonuclease | 74.4% | 0.63 |
| | | system | | FokI, catalytic domain | | |
| | | found in | | | | |
| | | round 1 | | | | |
| pfam02981 | 2 | known | RM | Restriction endonuclease | 75.0% | 0.64 |
| | | system | | FokI, recognition domain | | |
| | | found in | | | | |
| | | round 1 | | | | |
| pfam03235 | 2 | known | RM | Protein of unknown function | 78.8% | 0.38 |
| | | system | | DUF262 | | |
| | | found in | | | | |
| | | round 1, | | | | |
| | | putative | | | | |
| | | system | | | | |
| | | found in | | | | |
| | | round 1 | | | | |
| pfam03683 | 2 | known | TA | predicted | 88.3% | 0.54 |
| | | system | | | | |
| | | found in | | | | |
| | | round 1 | | | | |
| pfam03693 | 2 | known | TA | Bacterial antitoxin of ParD | 88.1% | 0.19 |
| | | system | | toxin-antitoxin type II system | | |
| | | found in | | and RHH | | |
| | | round 1 | | | | |
| pfam03750 | 2 | known | CRISPR | Protein of unknown function | 99.9% | 0.26 |
| | | system | | (DUF310) | | |
| | | found in | | | | |
| | | round 1 | | | | |
| pfam04255 | 2 | known | TA | Protein of unknown function | 69.0% | 0.36 |
| | | system | | (DUF433) | | |
| | | found in | | | | |
| | | round 1 | | | | |
| pfam04365 | 2 | known | TA | Ribonuclease toxin, BmT, of | 91.1% | 0.33 |
| | | system | | type II toxin-antitoxin system | | |
| | | found in | | | | |
| | | round 1 | | | | |
| pfam04411 | 2 | known | RM | PD-(D/E)XK nuclease | 84.0% | 0.57 |
| | | system | | superfamily | | |
| | | found in | | | | |
| | | round 1 | | | | |
| pfam05015 | 2 | known | TA | HigAB; RelE-like toxin of | 96.9% | 0.26 |
| | | system | | type II toxin-antitoxin system | | |
| | | found in | | HigB | | |
| | | round 1 | | | | |
| pfam05107 | 2 | known | CRISPR | CRISPR-associated protein | 98.5% | 0.41 |
| | | system | | Cas7 | | |
| | | found in | | | | |
| | | round 1 | | | | |
| pfam05168 | 2 | known | TA | HEPN domain | 84.9% | 0.36 |
| | | system | | | | |
| | | found in | | | | |
| | | round 1 | | | | |
| pfam05315 | 2 | known | RM | ICEA Protein | 98.7% | 0.17 |
| | | system | | | | |
| | | found in | | | | |
| | | round 1 | | | | |
| pfam06023 | 2 | known | CRISPR | Archaeal protein of unknown | 93.9% | 0.71 |
| | | system | | function (DUF911) | | |
| | | found in | | | | |
| | | round 1 | | | | |
| pfam06154 | 2 | known | TA | CbeA_antitoxin, type IV, | 97.2% | 0.11 |
| | | system | | cytoskeleton bundling- | | |
| | | found in | | enhancing factor A | | |
| | | round 1 | | | | |
| pfam06300 | 2 | known | RM | Tsp45I type II restriction | 97.1% | 0.33 |
| | | system | | enzyme | | |
| | | found in | | | | |
| | | round 1 | | | | |
| pfam06634 | 2 | known | RM, System | Protein of unknown function | 95.5% | 0.66 |
| | | system | around | (DUF1156) | | |
| | | found in | anchors | | | |
| | | round 1, | pfam06634, | | | |
| | | putative | COG1483, | | | |
| | | system | pfam12635 | | | |
| | | found in | | | | |
| | | round 1 | | | | |
| pfam06755 | 2 | known | TA | CbtA_toxin of type IV toxin- | 96.6% | 0.12 |
| | | system | | antitoxin system | | |
| | | found in | | | | |
| | | round 1 | | | | |
| pfam07362 | 2 | known | TA | Post-segregation antitoxin | 94.5% | 0.15 |
| | | system | | CcdA | | |
| | | found in | | | | |
| | | round 1 | | | | |
| pfam07510 | 2 | known | RM | Protein of unknown function | 72.8% | 0.29 |
| | | system | | (DUF1524) | | |
| | | found in | | | | |
| | | round 1, | | | | |
| | | putative | | | | |
| | | system | | | | |
| | | found in | | | | |
| | | round 1 | | | | |
| pfam07704 | 2 | known | TA | Rv0623-like transcription | 99.3% | 0.05 |
| | | system | | factor | | |
| | | found in | | | | |
| | | round 1 | | | | |
| pfam07832 | 2 | known | RM | Cfr10I/Bse634I restriction | 100.0% | 0.57 |
| | | system | | endonuclease | | |
| | | found in | | | | |
| | | round 1 | | | | |
| pfam08870 | 2 | known | DND | Domain of unknown function | 96.1% | 0.52 |
| | | system | | (DUF1832) | | |
| | | found in | | | | |
| | | round 1 | | | | |
| pfam08998 | 2 | known | TA | Bacterial epsilon antitoxin | 92.0% | 0.22 |
| | | system | | | | |
| | | found in | | | | |
| | | round 1 | | | | |
| pfam09015 | 2 | known | RM | NgoMIV restriction enzyme | 98.1% | 0.34 |
| | | system | | | | |
| | | found in | | | | |
| | | round 1 | | | | |
| pfam09126 | 2 | known | RM | Restriction endonuclease NaeI | 95.9% | 0.50 |
| | | system | | | | |
| | | found in | | | | |
| | | round 1 | | | | |
| pfam09226 | 2 | known | RM | Restriction endonuclease | 97.1% | 0.47 |
| | | system | | HincII | | |
| | | found in | | | | |
| | | round 1 | | | | |
| pfam09254 | 2 | known | RM | Restriction endonuclease | 78.3% | 0.73 |
| | | system | | FokI, C terminal | | |
| | | found in | | | | |
| | | round 1 | | | | |
| pfam09386 | 2 | known | TA | Antitoxin ParD | 92.1% | 0.58 |
| | | system | | | | |
| | | found in | | | | |
| | | round 1 | | | | |
| pfam09499 | 2 | known | RM | ApaLI-like restriction | 99.1% | 0.10 |
| | | system | | endonuclease | | |
| | | found in | | | | |
| | | round 1 | | | | |
| pfam09518 | 2 | known | RM | HindIII restriction | 84.8% | 0.46 |
| | | system | | endonuclease | | |
| | | found in | | | | |
| | | round 1 | | | | |
| pfam09519 | 2 | known | RM | Hind VP restriction | 93.8% | 0.56 |
| | | system | | endonuclease | | |
| | | found in | | | | |
| | | round 1 | | | | |
| pfam09521 | 2 | known | RM | NgoPII restriction | 96.4% | 0.18 |
| | | system | | endonuclease | | |
| | | found in | | | | |
| | | round 1 | | | | |
| pfam09545 | 2 | known | RM | AccI restriction endonuclease | 95.2% | 0.68 |
| | | system | | | | |
| | | found in | | | | |
| | | round 1 | | | | |
| pfam09553 | 2 | known | RM | Eco47II restriction | 97.6% | 0.36 |
| | | system | | endonuclease | | |
| | | found in | | | | |
| | | round 1 | | | | |
| pfam09554 | 2 | known | RM | HaeII restriction endonuclease | 97.5% | 0.33 |
| | | system | | | | |
| | | found in | | | | |
| | | round 1 | | | | |
| pfam09561 | 2 | known | RM | HpaII restriction | 82.0% | 0.33 |
| | | system | | endonuclease | | |
| | | found in | | | | |
| | | round 1 | | | | |
| pfam09564 | 2 | known | RM | NgoBV restriction | 99.6% | 0.13 |
| | | system | | endonuclease | | |
| | | found in | | | | |
| | | round 1 | | | | |
| pfam09566 | 2 | known | RM | SacI restriction endonuclease | 95.8% | 0.41 |
| | | system | | | | |
| | | found in | | | | |
| | | round 1 | | | | |
| pfam09570 | 2 | known | RM | SinI restriction endonuclease | 95.1% | 0.49 |
| | | system | | | | |
| | | found in | | | | |
| | | round 1 | | | | |
| pfam09711 | 2 | known | CRISPR | CRISPR-associated protein | 98.9% | 0.15 |
| | | system | | (Cas_Csn2) | | |
| | | found in | | | | |
| | | round 1 | | | | |
| pfam09722 | 2 | known | TA | Protein of unknown function | 92.7% | 0.16 |
| | | system | | (DUF2384) | | |
| | | found in | | | | |
| | | round 1 | | | | |
| pfam09907 | 2 | known | TA | HigAB | 97.8% | 0.18 |
| | | system | | | | |
| | | found in | | | | |
| | | round 1 | | | | |
| pfam09946 | 2 | known | TA | predicted | 97.9% | 0.16 |
| | | system | | | | |
| | | found in | | | | |
| | | round 1 | | | | |
| pfam09952 | 2 | known | TA | putative toxin has a | 94.2% | 0.13 |
| | | system | | nucleotidyl transferase | | |
| | | found in | | domain | | |
| | | round 1 | | | | |
| pfam10117 | 2 | known | RM | McrBC 5-methylcytosine | 75.9% | 0.40 |
| | | system | | restriction system component | | |
| | | found in | | | | |
| | | round 1 | | | | |
| pfam10884 | 2 | known | TA | Protein of unknown function | 80.7% | 0.44 |
| | | system | | (DUF2683) | | |
| | | found in | | | | |
| | | round 1 | | | | |
| pfam10959 | 2 | known | TA | Protein of unknown function | 91.3% | 0.33 |
| | | system | | (DUF2761) | | |
| | | found in | | | | |
| | | round 1 | | | | |
| pfam11463 | 2 | known | RM | R.HinP1I restriction | 90.9% | 0.36 |
| | | system | | endonuclease | | |
| | | found in | | | | |
| | | round 1 | | | | |
| pfam11564 | 2 | known | RM | Restriction endonuclease | 100.0% | 0.58 |
| | | system | | BpuJI-N terminal | | |
| | | found in | | | | |
| | | round 1 | | | | |
| pfam11848 | 2 | known | TA | contains PIN domain. Two | 95.9% | 0.54 |
| | | system | | small proteins | | |
| | | found in | | | | |
| | | round 1 | | | | |
| pfam11867 | 2 | known | RM | Domain of unknown function | 86.9% | 0.37 |
| | | system | | (DUF3387) | | |
| | | found in | | | | |
| | | round 1 | | | | |
| pfam12102 | 2 | known | RM | Domain of unknown function | 46.0% | 0.23 |
| | | system | | (DUF3578) | | |
| | | found in | | | | |
| | | round 1 | | | | |
| pfam12441 | 2 | known | TA | CopG antitoxin of type II | 89.9% | 0.46 |
| | | system | | toxin-antitoxin system | | |
| | | found in | | | | |
| | | round 1 | | | | |
| pfam12469 | 2 | known | CRISPR | CRISPR-associated protein | 98.2% | 0.70 |
| | | system | | | | |
| | | found in | | | | |
| | | round 1 | | | | |
| pfam12564 | 2 | known | RM | Type III | 95.3% | 0.25 |
| | | system | | restriction/modification | | |
| | | found in | | enzyme methylation subunit | | |
| | | round 1 | | | | |
| pfam12676 | 2 | known | TA | Protein of unknown function | 96.5% | 0.08 |
| | | system | | (DUF3796) | | |
| | | found in | | | | |
| | | round 1 | | | | |
| pfam13156 | 2 | known | RM | maybe a type IIG gene; | 82.5% | 0.19 |
| | | system | | Restriction endonuclease | | |
| | | found in | | | | |
| | | round 1 | | | | |
| pfam13337 | 2 | known | BREX | Putative ATP-dependent Lon | 91.8% | 0.56 |
| | | system | | protease | | |
| | | found in | | | | |
| | | round 1 | | | | |
| pfam13391 | 2 | known | RM | HNH_2, HNH endonuclease | 75.3% | 0.36 |
| | | system | | | | |
| | | found in | | | | |
| | | round 1 | | | | |
| pfam13395 | 2 | known | CRISPR | HNH endonuclease | 85.4% | 0.34 |
| | | system | | | | |
| | | found in | | | | |
| | | round 1 | | | | |
| pfam13643 | 2 | known | RM | Domain of unknown function | 70.9% | 0.44 |
| | | system | | (DUF4145) | | |
| | | found in | | | | |
| | | round 1 | | | | |
| pfam13957 | 2 | known | TA | Toxin YafO, type II toxin- | 79.6% | 0.11 |
| | | system | | antitoxin system | | |
| | | found in | | | | |
| | | round 1 | | | | |
| pfam14072 | 2 | known | DND | DndB, DNA-sulfur | 86.1% | 0.44 |
| | | system | | modification-associated | | |
| | | found in | | | | |
| | | round 1 | | | | |
| pfam14076 | 2 | known | TA | Domain of unknown function | 78.5% | 0.38 |
| | | system | | (DUF4258) | | |
| | | found in | | | | |
| | | round 1 | | | | |
| pfam14335 | 2 | known | RM | Domain of unknown function | 92.8% | 0.54 |
| | | system | | (DUF4391) | | |
| | | found in | | | | |
| | | round 1 | | | | |
| pfam14338 | 2 | known | RM | Mrr N-terminal domain | 87.8% | 0.27 |
| | | system | | | | |
| | | found in | | | | |
| | | round 1 | | | | |
| pfam14350 | 2 | known | TA | Nearby gene is HigA | 71.2% | 0.39 |
| | | system | | antitoxin | | |
| | | found in | | | | |
| | | round 1 | | | | |
| pfam14355 | 2 | known | Abi | Abortive infection C-terminus | 73.1% | 0.41 |
| | | system | | | | |
| | | found in | | | | |
| | | round 1 | | | | |
| pfam14367 | 2 | known | TA | Domain of unknown function | 98.0% | 0.17 |
| | | system | | (DUF4411) | | |
| | | found in | | | | |
| | | round 1 | | | | |
| pfam14384 | 2 | known | TA | BrnA antitoxin of type II | 88.9% | 0.26 |
| | | system | | toxin-antitoxin system | | |
| | | found in | | | | |
| | | round 1 | | | | |
| pfam14487 | 2 | known | TA | pmen type TA system | 88.7% | 0.22 |
| | | system | | | | |
| | | found in | | | | |
| | | round 1 | | | | |
| pfam14511 | 2 | known | RM | Type II restriction | 95.1% | 0.42 |
| | | system | | endonuclease EcoO109I | | |
| | | found in | | | | |
| | | round 1 | | | | |
| pfam14809 | 2 | known | tgtA | C1 domain of tRNA-guanine | 81.1% | 0.57 |
| | | system | (deazaguanine | transglycosylase dimerisation | | |
| | | found in | tRNA | | | |
| | | round 1 | modification) | | | |
| pfam15514 | 2 | known | RM | Restriction endonuclease ThaI | 95.1% | 0.33 |
| | | system | | | | |
| | | found in | | | | |
| | | round 1 | | | | |
| pfam15515 | 2 | known | RM | MvaI/BcnI restriction | 95.0% | 0.44 |
| | | system | | endonuclease family | | |
| | | found in | | | | |
| | | round 1 | | | | |
| pfam15723 | 2 | known | TA | Motility quorum-sensing | 81.1% | 0.19 |
| | | system | | regulator, toxin of MqsA | | |
| | | found in | | | | |
| | | round 1 | | | | |
| pfam15738 | 2 | known | TA | Bacterial toxin of type II | 87.0% | 0.22 |
| | | system | | toxin-antitoxin system, YafQ | | |
| | | found in | | | | |
| | | round 1 | | | | |
| pfam16277 | 2 | known | TA | Domain of unknown function | 87.5% | 0.22 |
| | | system | | (DUF4926) | | |
| | | found in | | | | |
| | | round 1 | | | | |
| pfam16565 | 2 | known | BREX | Lon Protease; Phospholipase | 85.5% | 0.74 |
| | | system | | D-like domain at C-terminus | | |
| | | found in | | of MIT | | |
| | | round 1 | | | | |
| pfam16592 | 2 | known | CRISPR | REC lobe of CRISPR- | 98.0% | 0.17 |
| | | system | | associated endonuclease Cas9 | | |
| | | found in | | | | |
| | | round 1 | | | | |
| pfam16593 | 2 | known | CRISPR | Bridge helix of CRISPR- | 97.8% | 0.14 |
| | | system | | associated endonuclease Cas9 | | |
| | | found in | | | | |
| | | round 1 | | | | |
| pfam16595 | 2 | known | CRISPR | PAM-interacting domain of | 99.4% | 0.14 |
| | | system | | CRISPR-associated | | |
| | | found in | | endonuclease Cas9 | | |
| | | round 1 | | | | |
| pfam16762 | 2 | known | TA | PIN domain; Ribbon-helix- | 96.6% | 0.38 |
| | | system | | helix domain | | |
| | | found in | | | | |
| | | round 1 | | | | |
| pfam16813 | 2 | known | CRISPR | CRISPR-associated protein | 98.8% | 0.28 |
| | | system | | Csn2 subfamily St | | |
| | | found in | | | | |
| | | round 1 | | | | |
| pfam16902 | 2 | known | RM | FokI; Type-2 restriction | 73.5% | 0.62 |
| | | system | | enzyme D3 domain | | |
| | | found in | | | | |
| | | round 1 | | | | |
| pfam16948 | 2 | known | TA | HipAB; Cell translocating | 73.9% | 0.56 |
| | | system | | kinase A N-terminus | | |
| | | found in | | | | |
| | | round 1 | | | | |
| pfam02384 | 2 | known | RM | N-6 DNA Methylase | 79.5% | 0.31 |
| | | system | | | | |
| | | found in | | | | |
| | | round 1 | | | | |
| pfam01541 | 2 | putative | single-gene | GIY-YIG catalytic domain | 38.0% | 0.12 |
| | | system | “system” of | | | |
| | | found in | pfam14267 | | | |
| | | round 1 | | | | |
| pfam01618 | 2 | putative | Zorya | ZorA; MotA/TolQ/ExbB | 66.5% | 0.06 |
| | | system | | proton channel family | | |
| | | found in | | | | |
| | | round 1 | | | | |
| pfam01863 | 2 | putative | single-gene | Protein of unknown function | 62.3% | 0.18 |
| | | system | “system” of | DUF45 | | |
| | | found in | COG1451 | | | |
| | | round 1 | | | | |
| pfam00691 | 2 | putative | Zorya | ZorB; OmpA family | 60.7% | 0.06 |
| | | system | | | | |
| | | found in | | | | |
| | | round 1 | | | | |
| pfam03174 | 2 | putative | System | Chitobiase/beta- | 41.9% | 0.24 |
| | | system | around | hexosaminidase C-terminal | | |
| | | found in | anchors | domain | | |
| | | round 1 | pfam06634, | | | |
| | | | COG1483, | | | |
| | | | pfam12635 | | | |
| pfam04383 | 2 | putative | single-gene | KilA-N domain | 43.1% | 0.31 |
| | | system | “system” of | | | |
| | | found in | pfam04383 | | | |
| | | round 1 | | | | |
| pfam04471 | 2 | putative | Zorya | ZorD (in some cases); | 66.7% | 0.31 |
| | | system | | Restriction endonuclease | | |
| | | found in | | | | |
| | | round 1 | | | | |
| pfam06250 | 2 | putative | single-gene | Protein of unknown function | 55.6% | 0.42 |
| | | system | “system”of | (DUF1016) | | |
| | | found in | COG4804 | | | |
| | | round 1 | | | | |
| pfam08878 | 2 | putative | Hachiman | HamA; Domain of unknown | 89.5% | 0.48 |
| | | system | | function (DUF1837) | | |
| | | found in | | | | |
| | | round 1 | | | | |
| pfam09414 | 2 | putative | System | RNA ligase | 34.5% | 0.39 |
| | | system | around anchor | | | |
| | | found in | pfam12476 | | | |
| | | round 1 | | | | |
| pfam09983 | 2 | putative | Wadjet | JetD; Uncharacterized protein | 87.5% | 0.45 |
| | | system | | conserved in bacteria C- | | |
| | | found in | | term(DUF2220) | | |
| | | round 1 | | | | |
| pfam10088 | 2 | putative | Lamassu | LmuB; Uncharacterized | 57.3% | 0.52 |
| | | system | | protein conserved in bacteria | | |
| | | found in | | (DUF2326) | | |
| | | round 1 | | | | |
| pfam11795 | 2 | putative | Wadjet | JetD; Uncharacterized protein | 95.3% | 0.40 |
| | | system | | conserved in bacteria N-term | | |
| | | found in | | (DUF3322) | | |
| | | round 1 | | | | |
| pfam11855 | 2 | putative | Wadjet | JetA; Protein of unknown | 97.6% | 0.31 |
| | | system | | function (DUF3375) | | |
| | | found in | | | | |
| | | round 1 | | | | |
| pfam12476 | 2 | putative | System | Protein of unknown function | 89.0% | 0.45 |
| | | system | around anchor | (DUF3696) | | |
| | | found in | pfam12476 | | | |
| | | round 1 | | | | |
| pfam12635 | 2 | putative | System | Protein of unknown function | 98.8% | 0.69 |
| | | system | around | (DUF3780) | | |
| | | found in | anchors | | | |
| | | round 1 | pfam06634, | | | |
| | | | COG1483, | | | |
| | | | pfam12635 | | | |
| pfam13020 | 2 | putative | System | Domain of unknown function | 52.6% | 0.41 |
| | | system | around | (DUF3883) | | |
| | | found in | anchors | | | |
| | | round 1 | pfam06634, | | | |
| | | | COG1483 | | | |
| pfam13175 | 2 | putative | Septu + System | PtuA; AAA ATPase domain | 73.9% | 0.37 |
| | | system | around anchor | | | |
| | | found in | pfam12476 | | | |
| | | round 1 | | | | |
| pfam13208 | 2 | putative | System | TerB N-terminal domain | 89.6% | 0.29 |
| | | system | around anchor | | | |
| | | found in | pfam13208 | | | |
| | | round 1 | | | | |
| pfam13304 | 2 | putative | Septu + System | PtuA; AAA domain, putative | 63.8% | 0.33 |
| | | system | around anchor | AbiEii toxin, Type IV TA | | |
| | | found in | pfam12476 | system | | |
| | | round 1 | | | | |
| pfam13476 | 2 | putative | Septu | PtuA; AAA domain | 47.1% | 0.24 |
| | | system | | | | |
| | | found in | | | | |
| | | round 1 | | | | |
| pfam13677 | 2 | putative | Zorya | ZorB; Membrane MotB of | 92.5% | 0.04 |
| | | system | | proton-channel complex | | |
| | | found in | | MotA/MotB | | |
| | | round 1 | | | | |
| pfam13835 | 2 | putative | Wadjet | JetB; Domain of unknown | 98.0% | 0.32 |
| | | system | | function (DUF4194) | | |
| | | found in | | | | |
| | | round 1 | | | | |
| pfam13910 | 2 | putative | single-gene | Domain of unknown function | 69.0% | 0.34 |
| | | system | “system” of | (DUF4209) | | |
| | | found in | pfam13910 | | | |
| | | round 1 | | | | |
| pfam14130 | 2 | putative | Lamassu | LmuA; Domain of unknown | 74.8% | 0.45 |
| | | system | | function (DUF4297) | | |
| | | found in | | | | |
| | | round 1 | | | | |
| pfam14267 | 2 | putative | single-gene | Domain of unknown function | 78.8% | 0.60 |
| | | system | “system” of | (DUF4357) | | |
| | | found in | pfam14267 | | | |
| | | round 1 | | | | |
| pfam14491 | 2 | putative | Septu- | Protein of unknown function | 92.4% | 0.29 |
| | | system | associated | (DUF4435) | | |
| | | found in | | | | |
| | | round 1 | | | | |
| pfam15611 | 2 | putative | Zorya | ZorC; EH_Signature domain | 98.3% | 0.55 |
| | | system | | | | |
| | | found in | | | | |
| | | round 1 | | | | |
| pfam15615 | 2 | putative | System | TerB-C domain | 92.1% | 0.29 |
| | | system | around anchor | | | |
| | | found in | pfam13208 | | | |
| | | round 1 | | | | |
| pfam13280 | 2 | putative | System | exists in subtypes; WYL | 57.6% | 0.19 |
| | | system | around | domain | | |
| | | found in | anchors | | | |
| | | round 1 | pfam06634, | | | |
| | | | COG1483 | | | |
| COG1483 | 2 | known | RM, System | Predicted ATPase, AAA + | | |
| | | system | around | superfamily | | |
| | | found in | anchors | | | |
| | | round 1, | pfam06634, | | | |
| | | putative | COG1483, | | | |
| | | system | pfam12635 | | | |
| | | found in | | | | |
| | | round 1 | | | | |
| COG0419 | 2 | putative | Lamassu | LmuB; SbcC, DNA repair | | |
| | | system | | exonuclease SbcCD ATPase | | |
| | | found in | | subunit | | |
| | | round 1 | | | | |
| COG3727 | 2 | known | RM | Vsr accompanying C | | |
| | | system | | methylase, auxilary to RM; | | |
| | | found in | | G:T-mismatch repair DNA | | |
| | | round 1 | | endonuclease, very short | | |
| | | | | patch repair protein | | |
| COG0840 | 2 | putative | Zorya | ZorA; Methyl-accepting | | |
| | | system | | chemotaxis protein | | |
| | | found in | | | | |
| | | round 1 | | | | |
| COG1196 | 2 | putative | Lamassu | LmuB; Smc, Chromosome | | |
| | | system | | segregation ATPase | | |
| | | found in | | | | |
| | | round 1 | | | | |
| COG1204 | 2 | putative | Lamassu- | Replicative superfamily II | | |
| | | system | associated | helicase | | |
| | | found in | | | | |
| | | round 1 | | | | |
| COG1360 | 2 | putative | Zorya | ZorB; Flagellar motor protein | | |
| | | system | | MotB | | |
| | | found in | | | | |
| | | round 1 | | | | |
| COG1451 | 2 | putative | single-gene | Predicted metal-dependent | | |
| | | system | “system” of | hydrolase | | |
| | | found in | COG1451 | | | |
| | | round 1 | | | | |
| COG2378 | 2 | putative | System | Predicted DNA-binding | | |
| | | system | around | transcriptional regulator | | |
| | | found in | anchors | YafY, contains an HTH and | | |
| | | round 1 | pfam06634, | WYL domains | | |
| | | | COG1483 | | | |
| COG2885 | 2 | putative | Zorya | ZorB; Outer membrane | | |
| | | system | | protein OmpA and related | | |
| | | found in | | peptidoglycan-associated | | |
| | | round 1 | | (lipo)proteins | | |
| COG4913 | 2 | putative | Wadjet | JetC; Uncharacterized protein, | | |
| | | system | | contains a C-terminal ATPase | | |
| | | found in | | domain | | |
| | | round 1 | | | | |
| COG4924 | 2 | putative | Wadjet | JetD; Uncharacterized protein | | |
| | | system | | | | |
| | | found in | | | | |
| | | round 1 | | | | |
| COG4938 | 2 | putative | System | Predicted ATPase | | |
| | | system | around anchor | | | |
| | | found in | pfam12476 | | | |
| | | round 1 | | | | |
| COG5293 | 2 | putative | Lamassu | LmuB; Uncharacterized | | |
| | | system | | protein YydD, contains | | |
| | | found in | | DUF2326 domain | | |
| | | round 1 | | (pfam10088) | | |

¹Makarova et al. J Bacteriol. 2011 November; 193(21): 6039-6056
²Goldfarb T, et al. (2015) BREX is a novel phage resistance system widespread in microbial genomes. EMBO J. EMBO Press; 34: 169-83
³Submitted

Identification of Pfams Enriched Near Defense Genes:

The genome sequences, gene annotations and taxonomy annotations of all publicly available sequenced bacterial and archaeal genomes were downloaded from the NCBI FTP site (ftp.ncbi.nih.gov/genomes/genbank/bacteria/ and ftp.ncbi.nih.gov/genomes/genbank/archaea/, respectively) on April 2016. pfam annotations for bacterial and archaeal genes were obtained from the Integrated Microbial Genomes (IMG) database (Markowitz V M, et al. (2012) IMG: the Integrated Microbial Genomes database and comparative analysis system. nucleic Acids Res. Oxford University Press; 40: D115-22) on December 2015, and cross-referenced to the genes in the genomes downloaded from NCBI using the locus_tag Genbank field. All pfams annotated in at least 20 genes (“members”) across the analyzed genomes (14,083 pfams) were scanned. For each pfam, the number of member genes for which a gene having an annotation of a known defense gene family (Table 1) was present in proximity (up to ten genes upstream and ten genes downstream) was recorded. The fraction of defense-associated members out of total members (“defense score”) was calculated per pfam. A second score (“defense context variability score”) was calculated for each pfam as follows: for each member gene occurring with at least one defense gene in proximity, a list of the proximal defense genes was recorded, and the fraction of unique lists out of total number of lists for that pfam represents the score (for example: if pfamX is found within 20 genes in our set, with 15 of them having Cas9 nearby and 5 having type I R-M nearby, the number of unique lists is 2, and the “defense context variability score” is 2/20=0.1). Pfams with defense score >=65% and defense context variability score >=0.1 were taken for further analysis. This list was supplemented with 35 non-pfam gene families that were predicted to be associated with defense by Makarova et al. (2011) ibid, as well as 23 pfams that were predicted in the same study but did not pass the thresholds above (Table 2).

TABLE 2
Scanned Families

| | Defense | Variability | | |
|---|---|---|---|---|
| Family | score | score | Family source | Classification |
| pfam10802 | 65.4% | 0.82 | Prediction cycle 1 | Likely mobilome |
| pfam10657 | 96.4% | 0.15 | Prediction cycle 1 | Likely non-defense function |
| pfam09226 | 94.3% | 0.30 | Prediction cycle 1 | Known system |
| pfam14809 | 70.3% | 0.58 | Prediction cycle 1 | Likely known system |
| pfam11572 | 92.1% | 0.23 | Prediction cycle 1 | Likely non-defense function |
| pfam14455 | 67.4% | 0.79 | Prediction cycle 1 | Likely mobilome |
| pfam07832 | 100.0% | 0.53 | Prediction cycle 1 | Known system |
| pfam10959 | 84.8% | 0.31 | Prediction cycle 1 | Likely known system |
| pfam04033 | 85.7% | 0.77 | Prediction cycle 1 | Possibly belongs to a new defense |
| | | | | system |
| pfam09629 | 70.9% | 0.26 | Prediction cycle 1 | Likely mobilome |
| pfam01304 | 74.6% | 0.41 | Prediction cycle 1 | Likely non-defense function |
| pfam11536 | 75.0% | 0.60 | Prediction cycle 1 | Possibly belongs to a new defense |
| | | | | system |
| pfam09545 | 80.6% | 0.70 | Prediction cycle 1 | Known system |
| pfam11564 | 100.0% | 0.48 | Prediction cycle 1 | Known system |
| pfam09566 | 95.8% | 0.38 | Prediction cycle 1 | Known system |
| pfam16902 | 69.9% | 0.45 | Prediction cycle 1 | Known system |
| pfam12942 | 78.2% | 0.13 | Prediction cycle 1 | Likely non-defense function |
| pfam02981 | 71.6% | 0.46 | Prediction cycle 1 | Known system |
| pfam16992 | 86.5% | 0.18 | Prediction cycle 1 | Likely non-defense function |
| pfam02980 | 71.1% | 0.44 | Prediction cycle 1 | Known system |
| pfam12962 | 67.0% | 0.34 | Prediction cycle 1 | Likely mobilome |
| pfam09518 | 76.8% | 0.43 | Prediction cycle 1 | Known system |
| pfam10473 | 78.3% | 0.18 | Prediction cycle 1 | Likely mobilome |
| pfam14466 | 84.6% | 0.28 | Prediction cycle 1 | Likely mobilome |
| pfam14456 | 78.3% | 0.34 | Prediction cycle 1 | Likely mobilome |
| pfam10876 | 66.7% | 0.40 | Prediction cycle 1 | Likely mobilome |
| pfam09543 | 67.9% | 0.24 | Prediction cycle 1 | Likely non-defense function |
| pfam09254 | 76.1% | 0.58 | Prediction cycle 1 | Known system |
| pfam09973 | 65.5% | 0.23 | Prediction cycle 1 | Likely non-defense function |
| pfam09570 | 92.3% | 0.42 | Prediction cycle 1 | Known system |
| pfam15514 | 95.1% | 0.29 | Prediction cycle 1 | Known system |
| pfam08078 | 66.7% | 0.36 | Prediction cycle 1 | Likely non-defense function |
| pfam09519 | 93.2% | 0.45 | Prediction cycle 1 | Known system |
| pfam09554 | 97.5% | 0.29 | Prediction cycle 1 | Known system |
| pfam11428 | 67.7% | 0.13 | Prediction cycle 1 | Likely non-defense function |
| pfam16813 | 98.8% | 0.20 | Prediction cycle 1 | Known system |
| pfam09895 | 71.4% | 0.18 | Prediction cycle 1 | Likely non-defense function |
| pfam11463 | 88.2% | 0.26 | Prediction cycle 1 | Known system |
| pfam09436 | 84.7% | 0.28 | Prediction cycle 1 | Likely mobilome |
| pfam10884 | 75.6% | 0.40 | Prediction cycle 1 | Known system |
| pfam10907 | 85.1% | 0.15 | Prediction cycle 1 | Likely mobilome |
| pfam12963 | 67.1% | 0.32 | Prediction cycle 1 | Likely mobilome |
| pfam16827 | 81.6% | 0.37 | Prediction cycle 1 | Likely non-defense function |
| pfam09871 | 66.0% | 0.16 | Prediction cycle 1 | Likely non-defense function |
| pfam12635 | 94.7% | 0.55 | Prediction cycle 1 | Possibly belongs to a new defense |
| | | | | system |
| pfam09126 | 95.5% | 0.40 | Prediction cycle 1 | Known system |
| pfam06023 | 93.9% | 0.69 | Prediction cycle 1 | Known system |
| pfam11645 | 67.2% | 0.21 | Prediction cycle 1 | Likely non-defense function |
| pfam12959 | 87.7% | 0.48 | Prediction cycle 1 | Likely mobilome |
| pfam06300 | 95.0% | 0.26 | Prediction cycle 1 | Known system |
| pfam16948 | 68.6% | 0.49 | Prediction cycle 1 | Known system |
| pfam09386 | 91.1% | 0.52 | Prediction cycle 1 | Known system |
| pfam09466 | 89.4% | 0.23 | Prediction cycle 1 | Likely mobilome |
| pfam16468 | 92.3% | 0.28 | Prediction cycle 1 | Likely mobilome |
| pfam09015 | 98.1% | 0.29 | Prediction cycle 1 | Known system |
| pfam09544 | 81.4% | 0.19 | Prediction cycle 1 | Possibly belongs to a new defense |
| | | | | system |
| pfam05315 | 98.1% | 0.13 | Prediction cycle 1 | Known system |
| pfam11426 | 66.1% | 0.36 | Prediction cycle 1 | Likely mobilome |
| pfam09553 | 97.3% | 0.29 | Prediction cycle 1 | Known system |
| pfam14462 | 81.4% | 0.53 | Prediction cycle 1 | Likely non-defense function |
| pfam12960 | 90.8% | 0.46 | Prediction cycle 1 | Likely mobilome |
| pfam12997 | 80.4% | 0.17 | Prediction cycle 1 | Likely non-defense function |
| pfam09521 | 95.3% | 0.15 | Prediction cycle 1 | Known system |
| pfam07015 | 69.5% | 0.61 | Prediction cycle 1 | Likely mobilome |
| pfam14457 | 71.2% | 0.51 | Prediction cycle 1 | Likely mobilome |
| pfam08998 | 91.0% | 0.19 | Prediction cycle 1 | Known system |
| pfam14511 | 93.2% | 0.34 | Prediction cycle 1 | Known system |
| pfam09946 | 97.7% | 0.12 | Prediction cycle 1 | Likely known system |
| pfam10398 | 93.2% | 0.26 | Prediction cycle 1 | Likely non-defense function |
| pfam16762 | 96.4% | 0.33 | Prediction cycle 1 | Known system |
| pfam03513 | 68.1% | 0.29 | Prediction cycle 1 | Likely non-defense function |
| pfam07820 | 68.8% | 0.41 | Prediction cycle 1 | Likely mobilome |
| pfam11320 | 69.8% | 0.27 | Prediction cycle 1 | Likely non-defense function |
| pfam16565 | 81.7% | 0.61 | Prediction cycle 1 | Known system |
| pfam11341 | 67.9% | 0.27 | Prediction cycle 1 | Likely non-defense function |
| pfam09561 | 80.1% | 0.25 | Prediction cycle 1 | Known system |
| pfam14386 | 71.5% | 0.45 | Prediction cycle 1 | Likely mobilome |
| pfam14192 | 89.7% | 0.30 | Prediction cycle 1 | Likely mobilome |
| pfam12617 | 73.3% | 0.24 | Prediction cycle 1 | Likely non-defense function |
| pfam14203 | 88.3% | 0.37 | Prediction cycle 1 | Likely mobilome |
| pfam07550 | 67.6% | 0.12 | Prediction cycle 1 | Likely non-defense function |
| pfam15515 | 93.8% | 0.37 | Prediction cycle 1 | Known system |
| pfam14101 | 76.9% | 0.11 | Prediction cycle 1 | Likely non-defense function |
| pfam16509 | 79.5% | 0.34 | Prediction cycle 1 | Likely mobilome |
| pfam08950 | 65.4% | 0.12 | Prediction cycle 1 | Likely non-defense function |
| pfam09274 | 70.3% | 0.39 | Prediction cycle 1 | Likely mobilome |
| pfam07764 | 78.8% | 0.19 | Prediction cycle 1 | Likely mobilome |
| pfam11083 | 80.5% | 0.11 | Prediction cycle 1 | Likely mobilome |
| pfam12957 | 68.2% | 0.48 | Prediction cycle 1 | Likely mobilome |
| pfam14194 | 73.8% | 0.29 | Prediction cycle 1 | Likely mobilome |
| pfam14461 | 67.1% | 0.35 | Prediction cycle 1 | Likely mobilome |
| pfam12599 | 72.6% | 0.55 | Prediction cycle 1 | Likely mobilome |
| pfam16734 | 66.7% | 0.39 | Prediction cycle 1 | Likely non-defense function |
| pfam14452 | 85.5% | 0.47 | Prediction cycle 1 | Likely non-defense function |
| pfam14193 | 80.3% | 0.31 | Prediction cycle 1 | Likely mobilome |
| pfam14195 | 77.6% | 0.39 | Prediction cycle 1 | Likely mobilome |
| pfam02697 | 88.4% | 0.38 | Prediction cycle 1 | Known system |
| pfam14217 | 67.9% | 0.30 | Prediction cycle 1 | Likely non-defense function |
| pfam15611 | 92.5% | 0.41 | Prediction cycle 1 | Possibly belongs to a new defense |
| | | | | system |
| pfam13910 | 65.1% | 0.27 | Prediction cycle 1 | Possibly belongs to a new defense |
| | | | | system |
| pfam12958 | 67.2% | 0.29 | Prediction cycle 1 | Likely mobilome |
| pfam14130 | 66.5% | 0.35 | Prediction cycle 1 | Possibly belongs to a new defense |
| | | | | system |
| pfam14460 | 65.9% | 0.30 | Prediction cycle 1 | Likely mobilome |
| pfam05626 | 83.4% | 0.23 | Prediction cycle 1 | Uncertain |
| pfam12664 | 72.1% | 0.17 | Prediction cycle 1 | Likely mobilome |
| pfam16560 | 73.7% | 0.11 | Prediction cycle 1 | Likely mobilome |
| pfam04796 | 69.8% | 0.42 | Prediction cycle 1 | Likely mobilome |
| pfam14271 | 66.0% | 0.15 | Prediction cycle 1 | Likely non-defense function |
| pfam08870 | 92.8% | 0.32 | Prediction cycle 1 | Known system |
| pfam11211 | 65.2% | 0.29 | Prediction cycle 1 | Likely mobilome |
| pfam12469 | 97.8% | 0.67 | Prediction cycle 1 | Known system |
| pfam05037 | 74.5% | 0.21 | Prediction cycle 1 | Likely mobilome |
| pfam06666 | 78.2% | 0.32 | Prediction cycle 1 | Likely mobilome |
| pfam06868 | 67.3% | 0.30 | Prediction cycle 1 | Likely mobilome |
| pfam08989 | 73.7% | 0.28 | Prediction cycle 1 | Uncertain |
| pfam04411 | 69.9% | 0.54 | Prediction cycle 1 | Likely known system |
| pfam11225 | 73.4% | 0.16 | Prediction cycle 1 | Likely non-defense function |
| pfam14350 | 68.9% | 0.31 | Prediction cycle 1 | Known system |
| pfam14335 | 91.7% | 0.45 | Prediction cycle 1 | Possibly belongs to a new defense |
| | | | | system |
| pfam12476 | 72.9% | 0.41 | Prediction cycle 1 | Possibly belongs to a new defense |
| | | | | system |
| pfam12750 | 77.7% | 0.31 | Prediction cycle 1 | Likely mobilome |
| pfam12564 | 94.5% | 0.19 | Prediction cycle 1 | Known system |
| pfam14198 | 70.8% | 0.44 | Prediction cycle 1 | Likely mobilome |
| pfam13872 | 71.6% | 0.45 | Prediction cycle 1 | Likely mobilome |
| pfam06634 | 91.9% | 0.50 | Prediction cycle 1 | Possibly belongs to a new defense |
| | | | | system |
| pfam13871 | 72.8% | 0.46 | Prediction cycle 1 | Likely mobilome |
| pfam14283 | 81.6% | 0.35 | Prediction cycle 1 | Likely mobilome |
| pfam10987 | 70.9% | 0.14 | Prediction cycle 1 | Likely non-defense function |
| pfam08878 | 86.8% | 0.38 | Prediction cycle 1 | Possibly belongs to a new defense |
| | | | | system |
| pfam13208 | 83.0% | 0.24 | Prediction cycle 1 | Possibly belongs to a new defense |
| | | | | system |
| pfam09509 | 68.3% | 0.39 | Prediction cycle 1 | Likely known system |
| pfam12358 | 70.0% | 0.34 | Prediction cycle 1 | Likely mobilome |
| pfam15723 | 76.6% | 0.17 | Prediction cycle 1 | Known system |
| pfam10980 | 78.5% | 0.22 | Prediction cycle 1 | Possibly belongs to a new defense |
| | | | | system |
| pfam15615 | 85.9% | 0.23 | Prediction cycle 1 | Possibly belongs to a new defense |
| | | | | system |
| pfam08775 | 70.6% | 0.23 | Prediction cycle 1 | Likely mobilome |
| pfam08814 | 70.1% | 0.48 | Prediction cycle 1 | Possibly belongs to a new defense |
| | | | | system |
| pfam11848 | 82.8% | 0.53 | Prediction cycle 1 | Likely known system |
| pfam12441 | 81.8% | 0.42 | Prediction cycle 1 | Known system |
| pfam14267 | 72.7% | 0.54 | Prediction cycle 1 | Possibly belongs to a new defense |
| | | | | system |
| pfam14065 | 65.4% | 0.14 | Prediction cycle 1 | Likely mobilome |
| pfam13101 | 70.3% | 0.25 | Prediction cycle 1 | Likely mobilome |
| pfam01905 | 99.0% | 0.37 | Prediction cycle 1 | Known system |
| pfam13351 | 67.1% | 0.25 | Prediction cycle 1 | Likely mobilome |
| pfam13337 | 89.2% | 0.44 | Prediction cycle 1 | Known system |
| pfam11103 | 68.9% | 0.36 | Prediction cycle 1 | Likely non-defense function |
| pfam12083 | 85.2% | 0.20 | Prediction cycle 1 | Likely mobilome |
| pfam09035 | 73.8% | 0.17 | Prediction cycle 1 | Likely mobilome |
| pfam13707 | 93.3% | 0.26 | Prediction cycle 1 | Likely known system |
| pfam07638 | 67.4% | 0.17 | Prediction cycle 1 | Likely non-defense function |
| pfam08869 | 72.6% | 0.43 | Prediction cycle 1 | Possibly belongs to a new defense |
| | | | | system |
| pfam14355 | 67.4% | 0.33 | Prediction cycle 1 | Known system |
| pfam03750 | 99.9% | 0.25 | Prediction cycle 1 | Known system |
| pfam06892 | 71.1% | 0.12 | Prediction cycle 1 | Likely mobilome |
| pfam14076 | 73.9% | 0.33 | Prediction cycle 1 | Likely known system |
| pfam07515 | 77.4% | 0.20 | Prediction cycle 1 | Likely mobilome |
| pfam02920 | 67.7% | 0.17 | Prediction cycle 1 | Likely mobilome |
| pfam10134 | 72.8% | 0.34 | Prediction cycle 1 | Likely mobilome |
| pfam11140 | 71.2% | 0.13 | Prediction cycle 1 | Likely non-defense function |
| pfam01881 | 90.7% | 0.40 | Prediction cycle 1 | Known system |
| pfam14072 | 80.6% | 0.31 | Prediction cycle 1 | Known system |
| pfam10552 | 72.0% | 0.11 | Prediction cycle 1 | Likely mobilome |
| pfam09952 | 92.9% | 0.11 | Prediction cycle 1 | Known system |
| pfam06290 | 87.1% | 0.12 | Prediction cycle 1 | Likely mobilome |
| pfam16277 | 85.0% | 0.19 | Prediction cycle 1 | Likely known system |
| pfam14367 | 97.4% | 0.14 | Prediction cycle 1 | Known system |
| pfam03683 | 85.0% | 0.46 | Prediction cycle 1 | Known system |
| pfam15937 | 96.2% | 0.14 | Prediction cycle 1 | Likely mobilome |
| pfam13156 | 79.4% | 0.16 | Prediction cycle 1 | Likely known system |
| pfam06952 | 84.0% | 0.12 | Prediction cycle 1 | Likely mobilome |
| pfam06953 | 78.6% | 0.10 | Prediction cycle 1 | Likely non-defense function |
| pfam14487 | 82.1% | 0.18 | Prediction cycle 1 | Known system |
| pfam05107 | 98.5% | 0.35 | Prediction cycle 1 | Known system |
| pfam13711 | 92.8% | 0.32 | Prediction cycle 1 | Likely mobilome |
| pfam10554 | 73.2% | 0.12 | Prediction cycle 1 | Likely mobilome |
| pfam07514 | 76.0% | 0.21 | Prediction cycle 1 | Likely mobilome |
| pfam05284 | 73.1% | 0.36 | Prediction cycle 1 | Likely mobilome |
| pfam13154 | 68.0% | 0.28 | Prediction cycle 1 | Likely mobilome |
| pfam14261 | 66.3% | 0.35 | Prediction cycle 1 | Likely mobilome |
| pfam07128 | 96.0% | 0.13 | Prediction cycle 1 | Likely mobilome |
| pfam08273 | 79.8% | 0.13 | Prediction cycle 1 | Likely mobilome |
| pfam13395 | 81.0% | 0.25 | Prediction cycle 1 | Known system |
| pfam06406 | 79.1% | 0.16 | Prediction cycle 1 | Likely mobilome |
| pfam14338 | 84.2% | 0.23 | Prediction cycle 1 | Known system |
| pfam10117 | 65.1% | 0.35 | Prediction cycle 1 | Known system |
| pfam13643 | 66.4% | 0.35 | Prediction cycle 1 | Likely known system |
| pfam08401 | 69.4% | 0.33 | Prediction cycle 1 | Likely mobilome |
| pfam09907 | 97.4% | 0.14 | Prediction cycle 1 | Known system |
| pfam13148 | 68.0% | 0.25 | Prediction cycle 1 | Uncertain |
| pfam07275 | 75.3% | 0.14 | Prediction cycle 1 | Likely mobilome |
| pfam15738 | 85.6% | 0.18 | Prediction cycle 1 | Known system |
| pfam13708 | 75.2% | 0.11 | Prediction cycle 1 | Uncertain |
| pfam14384 | 84.4% | 0.22 | Prediction cycle 1 | Known system |
| pfam07362 | 93.8% | 0.12 | Prediction cycle 1 | Known system |
| pfam03288 | 70.3% | 0.13 | Prediction cycle 1 | Likely mobilome |
| pfam05015 | 96.4% | 0.21 | Prediction cycle 1 | Known system |
| pfam13310 | 68.4% | 0.36 | Prediction cycle 1 | Likely known system |
| pfam11867 | 78.7% | 0.29 | Prediction cycle 1 | Known system |
| pfam05168 | 82.0% | 0.31 | Prediction cycle 1 | Likely known system |
| pfam04365 | 80.6% | 0.28 | Prediction cycle 1 | Known system |
| pfam13391 | 67.8% | 0.28 | Prediction cycle 1 | Known system |
| pfam13362 | 76.6% | 0.19 | Prediction cycle 1 | Likely mobilome |
| pfam08706 | 65.4% | 0.16 | Prediction cycle 1 | Likely mobilome |
| pfam08774 | 69.9% | 0.11 | Prediction cycle 1 | Likely mobilome |
| pfam03693 | 86.8% | 0.16 | Prediction cycle 1 | Known system |
| pfam04255 | 66.4% | 0.32 | Prediction cycle 1 | Likely known system |
| pfam13744 | 87.2% | 0.15 | Prediction cycle 1 | Known system |
| pfam06067 | 76.7% | 0.15 | Prediction cycle 1 | Possibly belongs to a new defense |
| | | | | system |
| pfam14657 | 67.2% | 0.11 | Prediction cycle 1 | Likely mobilome |
| pfam15919 | 75.6% | 0.26 | Prediction cycle 1 | Likely non-defense function |
| pfam04221 | 84.2% | 0.18 | Prediction cycle 1 | Known system |
| pfam05973 | 78.5% | 0.17 | Prediction cycle 1 | Known system |
| pfam03235 | 66.7% | 0.28 | Prediction cycle 1 | Possibly belongs to a new defense |
| | | | | system |
| pfam13635 | 68.0% | 0.19 | Prediction cycle 1 | Possibly belongs to a new defense |
| | | | | system |
| pfam11657 | 78.9% | 0.37 | Prediction cycle 1 | Likely mobilome |
| pfam14662 | 72.0% | 0.53 | Prediction cycle 1 | Likely non-defense function |
| pfam04313 | 90.7% | 0.23 | Prediction cycle 1 | Known system |
| pfam08130 | 65.0% | 0.31 | Prediction cycle 1 | Likely non-defense function |
| pfam09116 | 94.7% | 0.39 | Prediction cycle 1 | Likely mobilome |
| pfam04582 | 85.1% | 0.19 | Prediction cycle 1 | Likely mobilome |
| pfam04875 | 79.3% | 0.26 | Prediction cycle 1 | Likely mobilome |
| pfam11592 | 68.2% | 0.31 | Prediction cycle 1 | Likely non-defense function |
| pfam09552 | 92.7% | 0.47 | Prediction cycle 1 | Known system |
| pfam10892 | 93.8% | 0.29 | Prediction cycle 1 | Likely mobilome |
| pfam09567 | 72.7% | 0.53 | Prediction cycle 1 | Known system |
| pfam02691 | 78.6% | 0.12 | Prediction cycle 1 | Likely non-defense function |
| pfam08046 | 78.9% | 0.10 | Prediction cycle 1 | Likely non-defense function |
| pfam11570 | 71.0% | 0.24 | Prediction cycle 1 | Likely non-defense function |
| pfam10083 | 81.9% | 0.19 | Prediction cycle 1 | Likely mobilome |
| pfam02048 | 68.5% | 0.44 | Prediction cycle 1 | Likely non-defense function |
| pfam13604 | 66.3% | 0.10 | Prediction cycle 1 | Possibly belongs to a new defense |
| | | | | system |
| pfam11770 | 73.5% | 0.32 | Prediction cycle 1 | Likely non-defense function |
| pfam06709 | 86.8% | 0.36 | Prediction cycle 1 | Possibly belongs to a new defense |
| | | | | system |
| pfam16790 | 84.2% | 0.50 | Prediction cycle 1 | Likely mobilome |
| pfam04604 | 65.0% | 0.13 | Prediction cycle 1 | Likely non-defense function |
| pfam12113 | 66.1% | 0.17 | Prediction cycle 1 | Likely mobilome |
| pfam14459 | 66.7% | 0.68 | Prediction cycle 1 | Likely mobilome |
| pfam12703 | 88.5% | 0.28 | Prediction cycle 1 | Known system |
| pfam02384 | 77.6% | 0.22 | Prediction cycle 1 | Known system |
| pfam16848 | 80.2% | 0.15 | Prediction cycle 1 | Likely non-defense function |
| pfam11056 | 76.2% | 0.31 | Prediction cycle 1 | Likely mobilome |
| pfam07339 | 95.1% | 0.36 | Prediction cycle 1 | Likely mobilome |
| pfam14562 | 96.9% | 0.39 | Prediction cycle 1 | Known system |
| pfam09185 | 82.9% | 0.14 | Prediction cycle 1 | Likely non-defense function |
| pfam12493 | 82.3% | 0.32 | Prediction cycle 1 | Likely mobilome |
| pfam16831 | 68.0% | 0.28 | Prediction cycle 1 | Likely non-defense function |
| pfam01818 | 75.0% | 0.50 | Prediction cycle 1 | Likely mobilome |
| pfam11554 | 67.2% | 0.69 | Prediction cycle 1 | Likely mobilome |
| pfam09504 | 97.8% | 0.34 | Prediction cycle 1 | Known system |
| pfam16243 | 100.0% | 0.22 | Prediction cycle 1 | Likely mobilome |
| pfam16280 | 90.9% | 0.34 | Prediction cycle 1 | Likely known system |
| pfam09020 | 98.5% | 0.14 | Prediction cycle 1 | Likely mobilome |
| pfam09562 | 80.0% | 0.55 | Prediction cycle 1 | Known system |
| pfam07154 | 75.5% | 0.45 | Prediction cycle 1 | Possibly belongs to a new defense |
| | | | | system |
| pfam06443 | 77.1% | 0.12 | Prediction cycle 1 | Likely non-defense function |
| pfam03295 | 100.0% | 0.50 | Prediction cycle 1 | Likely mobilome |
| pfam13128 | 68.4% | 0.15 | Prediction cycle 1 | Likely mobilome |
| pfam11504 | 74.8% | 0.18 | Prediction cycle 1 | Likely non-defense function |
| pfam12136 | 98.2% | 0.14 | Prediction cycle 1 | Likely non-defense function |
| pfam07042 | 74.7% | 0.50 | Prediction cycle 1 | Likely mobilome |
| pfam11459 | 87.6% | 0.24 | Prediction cycle 1 + | Likely known system |
| | | | Makarova et al. (2011)⁴ | |
| pfam11726 | 79.5% | 0.12 | Prediction cycle 1 + | Likely mobilome |
| | | | Makarova et al. (2011) | |
| pfam01954 | 87.5% | 0.54 | Prediction cycle 1 + | Known system |
| | | | Makarova et al. (2011) | |
| pfam03428 | 86.2% | 0.26 | Prediction cycle 1 + | Likely mobilome |
| | | | Makarova et al. (2011) | |
| pfam06924 | 91.9% | 0.19 | Prediction cycle 1 + | Likely mobilome |
| | | | Makarova et al. (2011) | |
| pfam07183 | 77.6% | 0.59 | Prediction cycle 1 + | Likely mobilome |
| | | | Makarova et al. (2011) | |
| pfam11800 | 84.5% | 0.26 | Prediction cycle 1 + | Likely mobilome |
| | | | Makarova et al. (2011) | |
| pfam09722 | 90.4% | 0.12 | Prediction cycle 1 + | Known system |
| | | | Makarova et al. (2011) | |
| pfam11994 | 72.6% | 0.39 | Prediction cycle 1 + | Possibly belongs to a new defense |
| | | | Makarova et al. (2011) | system |
| pfam12101 | 84.6% | 0.19 | Prediction cycle 1 + | Likely mobilome |
| | | | Makarova et al. (2011) | |
| pfam08900 | 90.5% | 0.17 | Prediction cycle 1 +30 | Likely mobilome |
| | | | Makarova et al. (2011) | |
| pfam10074 | 83.5% | 0.24 | Prediction cycle 1 + | Likely mobilome |
| | | | Makarova et al. (2011) | |
| pfam11000 | 71.8% | 0.22 | Prediction cycle 1 + | Likely mobilome |
| | | | Makarova et al. (2011) | |
| pfam11284 | 86.2% | 0.21 | Prediction cycle 1 + | Likely mobilome |
| | | | Makarova et al. (2011) | |
| pfam11358 | 95.8% | 0.17 | Prediction cycle 1 + | Likely mobilome |
| | | | Makarova et al. (2011) | |
| pfam11679 | 93.3% | 0.18 | Prediction cycle 1 + | Likely mobilome |
| | | | Makarova et al. (2011) | |
| pfam12553 | 86.9% | 0.19 | Prediction cycle 1 + | Likely mobilome |
| | | | Makarova et al. (2011) | |
| COG4688 | | | Makarova et al. (2011) | Likely mobilome |
| COG4190 | | | Makarova et al. (2011) | Likely known system |
| COG5428 | | | Makarova et al. (2011) | Likely known system |
| COG3756 | | | Makarova et al. (2011) | Likely mobilome |
| COG4185 | | | Makarova et al. (2011) | Likely non-defense function |
| COG4854 | | | Makarova et al. (2011) | Likely non-defense function |
| COG5532 | | | Makarova et al. (2011) | Likely mobilome |
| COG5545 | | | Makarova et al. (2011) | Likely mobilome |
| COG5658 | | | Makarova et al. (2011) | Likely non-defense function |
| pfam01724 | 64.0% | 0.34 | Makarova et al. (2011) | Likely non-defense function |
| pfam03432 | 62.3% | 0.27 | Makarova et al. (2011) | Likely mobilome |
| pfam09588 | 40.7% | 0.25 | Makarova et al. (2011) | Likely mobilome |
| pfam11238 | 37.1% | 0.10 | Makarova et al. (2011) | Likely non-defense function |
| COG1205 | | | Makarova et al. (2011) | Possibly belongs to a new defense |
| | | | | system |
| COG4913 | | | Makarova et al. (2011) | Possibly belongs to a new defense |
| | | | | system |
| COG4924 | | | Makarova et al. (2011) | Possibly belongs to a new defense |
| | | | | system |
| pfam04383 | 32.4% | 0.28 | Makarova et al. (2011) | Possibly belongs to a new defense |
| | | | | system |
| COG1112 | | | Makarova et al. (2011) | Uncertain |
| COG1373 | | | Makarova et al. (2011) | Uncertain |
| COG2865 | | | Makarova et al. (2011) | Uncertain |
| COG3598 | | | Makarova et al. (2011) | Likely mobilome |
| pfam10543 | 55.9% | 0.26 | Makarova et al. (2011) | Uncertain |
| pfam11185 | 50.8% | 0.35 | Makarova et al. (2011) | Uncertain |
| pfam09823 | 69.1% | 0.52 | Makarova et al. (2011) | Likely known system |
| COG5519 | | | Makarova et al. (2011) | Likely mobilome |
| pfam10711 | 29.6% | 0.18 | Makarova et al. (2011) | Likely mobilome |
| COG1483 | | | Makarova et al. (2011) | Possibly belongs to a new defense |
| | | | | system |
| COG3586 | | | Makarova et al. (2011) | Possibly belongs to a new defense |
| | | | | system |
| COG3950 | | | Makarova et al. (2011) | Possibly belongs to a new defense |
| | | | | system |
| COG4804 | | | Makarova et al. (2011) | Possibly belongs to a new defense |
| | | | | system |
| pfam01541 | 31.4% | 0.10 | Makarova et al. (2011) | Uncertain |
| COG1475 | | | Makarova et al. (2011) | Likely mobilome |
| COG0507 | | | Makarova et al. (2011) | Likely non-defense function |
| COG0553 | | | Makarova et al. (2011) | Possibly belongs to a new defense |
| | | | | system |
| COG1192 | | | Makarova et al. (2011) | Likely mobilome |
| COG1451 | | | Makarova et al. (2011) | Possibly belongs to a new defense |
| | | | | system |
| COG3727 | | | Makarova et al. (2011) | Known system |
| pfam01464 | 22.2% | 0.10 | Makarova et al. (2011) | Likely mobilome |
| pfam02876 | 60.9% | 0.01 | Makarova et al. (2011) | Likely non-defense function |
| pfam03837 | 33.5% | 0.12 | Makarova et al. (2011) | Likely mobilome |
| pfam08861 | 30.3% | 0.09 | Makarova et al. (2011) | Likely known system |
| pfam09199 | 92.0% | 0.00 | Makarova et al. (2011) | Likely non-defense function |
| pfam09954 | 21.0% | 0.31 | Makarova et al. (2011) | Possibly belongs to a new defense |
| | | | | system |
| COG3505 | | | Makarova et al. (2011) | Likely mobilome |
| COG3843 | | | Makarova et al. (2011) | Likely mobilome |
| COG4227 | | | Makarova et al. (2011) | Likely mobilome |
| COG4646 | | | Makarova et al. (2011) | Likely mobilome |
| COG4930 | | | Makarova et al. (2011) | Known system |
| COG5489 | | | Makarova et al. (2011) | Likely mobilome |
| pfam07895 | 42.0% | 0.03 | Makarova et al. (2011) | Likely non-defense function |
| pfam08849 | 95.2% | 0.39 | Makarova et al. (2011) | Known system |
| pfam09002 | 46.1% | 0.50 | Makarova et al. (2011) | Known system |
| pfam11198 | 80.0% | 0.14 | Makarova et al. (2011) | Likely mobilome |
| pfam11455 | 97.1% | 0.10 | Makarova et al. (2011) | Known system |
| pfam11985 | 22.7% | 0.18 | Makarova et al. (2011) | Likely mobilome |
| COG1479 | | | Makarova et al. (2011) | Known system |
| COG3472 | | | Makarova et al. (2011) | Known system |
| pfam10923 | 93.1% | 0.28 | Makarova et al. (2011) | Possibly belongs to a new defense |
| | | | | system |
| pfam12495 | 85.7% | 0.17 | Prediction cycle 2 | Likely non-defense function |
| pfam04787 | 100.0% | 1.00 | Prediction cycle 2 | Likely mobilome |
| pfam15541 | 69.0% | 0.40 | Prediction cycle 2 | Likely mobilome |
| pfam04778 | 68.8% | 0.36 | Prediction cycle 2 | Likely non-defense function |
| pfam07199 | 69.7% | 0.26 | Prediction cycle 2 | Likely non-defense function |
| pfam10790 | 68.4% | 0.65 | Prediction cycle 2 | Likely mobilome |
| pfam09131 | 79.5% | 0.19 | Prediction cycle 2 | Likely non-defense function |
| pfam16445 | 66.7% | 0.19 | Prediction cycle 2 | Likely non-defense function |
| pfam11519 | 69.0% | 0.28 | Prediction cycle 2 | Likely non-defense function |
| pfam11516 | 95.3% | 0.17 | Prediction cycle 2 | Likely non-defense function |
| pfam16904 | 76.5% | 0.10 | Prediction cycle 2 | Likely non-defense function |
| pfam07046 | 90.4% | 0.11 | Prediction cycle 2 | Likely non-defense function |
| pfam16436 | 86.8% | 0.11 | Prediction cycle 2 | Likely non-defense function |
| pfam14590 | 72.2% | 0.15 | Prediction cycle 2 | Likely non-defense function |
| pfam12772 | 69.8% | 0.22 | Prediction cycle 2 | Likely non-defense function |
| pfam11263 | 98.3% | 0.10 | Prediction cycle 2 | Likely non-defense function |
| pfam10913 | 69.7% | 0.11 | Prediction cycle 2 | Likely mobilome |
| pfam16390 | 68.8% | 0.13 | Prediction cycle 2 | Uncertain |
| pfam16847 | 65.0% | 0.38 | Prediction cycle 2 | Likely non-defense function |
| pfam11565 | 92.0% | 0.11 | Prediction cycle 2 | Likely non-defense function |
| pfam03857 | 65.7% | 0.52 | Prediction cycle 2 | Likely non-defense function |
| pfam13497 | 92.2% | 0.19 | Prediction cycle 2 | Likely mobilome |
| pfam08048 | 85.3% | 0.33 | Prediction cycle 2 | Likely mobilome |
| pfam09264 | 83.5% | 0.11 | Prediction cycle 2 | Likely non-defense function |
| pfam05946 | 72.9% | 0.13 | Prediction cycle 2 | Likely non-defense function |
| pfam09930 | 97.8% | 0.16 | Prediction cycle 2 | Likely non-defense function |
| pfam16163 | 68.5% | 0.68 | Prediction cycle 2 | Likely non-defense function |
| pfam16754 | 75.7% | 0.29 | Prediction cycle 2 | Known system |
| pfam16416 | 65.2% | 0.48 | Prediction cycle 2 | Possibly belongs to a new defense |
| | | | | system |
| pfam06613 | 82.2% | 0.36 | Prediction cycle 2 | Likely mobilome |
| pfam16479 | 88.9% | 0.11 | Prediction cycle 2 | Likely non-defense function |
| pfam05969 | 65.3% | 0.40 | Prediction cycle 2 | Likely non-defense function |
| pfam15538 | 66.0% | 0.10 | Prediction cycle 2 | Likely non-defense function |
| pfam10865 | 68.9% | 0.41 | Prediction cycle 2 | Likely non-defense function |
| pfam07828 | 85.5% | 0.12 | Prediction cycle 2 | Likely non-defense function |
| pfam11580 | 85.6% | 0.10 | Prediction cycle 2 | Likely non-defense function |
| pfam08017 | 68.3% | 0.10 | Prediction cycle 2 | Likely non-defense function |
| pfam05265 | 88.7% | 0.12 | Prediction cycle 2 | Likely mobilome |
| pfam13009 | 66.7% | 0.53 | Prediction cycle 2 | Likely mobilome |
| pfam09016 | 65.9% | 0.19 | Prediction cycle 2 | Likely non-defense function |
| pfam10726 | 67.3% | 0.29 | Prediction cycle 2 | Likely non-defense function |
| pfam10130 | 69.0% | 0.71 | Prediction cycle 2 | Known system |
| pfam10616 | 78.4% | 0.10 | Prediction cycle 2 | Likely non-defense function |
| pfam13314 | 69.8% | 0.11 | Prediction cycle 2 | Likely non-defense function |
| pfam04485 | 65.1% | 0.35 | Prediction cycle 2 | Possibly belongs to a new defense |
| | | | | system |
| pfam12655 | 65.6% | 0.12 | Prediction cycle 2 | Likely non-defense function |
| pfam13079 | 69.0% | 0.11 | Prediction cycle 2 | Likely non-defense function |
| pfam14275 | 67.6% | 0.13 | Prediction cycle 2 | Likely non-defense function |
| pfam11686 | 70.8% | 0.11 | Prediction cycle 2 | Likely non-defense function |
| pfam11397 | 67.0% | 0.13 | Prediction cycle 2 | Likely non-defense function |
| pfam11796 | 65.0% | 0.59 | Prediction cycle 2 | Possibly belongs to a new defense |
| | | | | system |
| pfam09000 | 66.3% | 0.28 | Prediction cycle 2 | Likely non-defense function |
| pfam14453 | 65.8% | 0.12 | Prediction cycle 2 | Likely non-defense function |
| pfam13651 | 85.8% | 0.46 | Prediction cycle 2 | Known system |
| pfam11173 | 66.6% | 0.11 | Prediction cycle 2 | Likely non-defense function |
| pfam16162 | 70.6% | 0.46 | Prediction cycle 2 | Possibly belongs to a new defense |
| | | | | system |
| pfam12975 | 68.1% | 0.11 | Prediction cycle 2 | Likely non-defense function |
| pfam14133 | 71.1% | 0.18 | Prediction cycle 2 | Likely non-defense function |
| pfam16082 | 65.4% | 0.15 | Prediction cycle 2 | Likely mobilome |
| pfam16223 | 66.3% | 0.11 | Prediction cycle 2 | Likely non-defense function |
| pfam12545 | 65.6% | 0.11 | Prediction cycle 2 | Likely non-defense function |
| pfam09664 | 67.9% | 0.50 | Prediction cycle 2 | Possibly belongs to a new defense |
| | | | | system |
| pfam11813 | 79.5% | 0.10 | Prediction cycle 2 | Likely non-defense function |
| pfam11149 | 69.5% | 0.52 | Prediction cycle 2 | Likely mobilome |
| pfam16263 | 76.4% | 0.35 | Prediction cycle 2 | Possibly belongs to a new defense |
| | | | | system |
| pfam12119 | 72.8% | 0.11 | Prediction cycle 2 | Likely non-defense function |
| pfam10893 | 91.7% | 0.13 | Prediction cycle 2 | Likely mobilome |
| pfam14790 | 67.2% | 0.11 | Prediction cycle 2 | Likely non-defense function |
| pfam12668 | 68.0% | 0.38 | Prediction cycle 2 | Possibly belongs to a new defense |
| | | | | system |
| pfam14082 | 65.3% | 0.57 | Prediction cycle 2 | Possibly belongs to a new defense |
| | | | | system |
| pfam06698 | 79.9% | 0.12 | Prediction cycle 2 | Likely non-defense function |
| pfam07509 | 65.6% | 0.12 | Prediction cycle 2 | Likely non-defense function |
| pfam07323 | 77.8% | 0.12 | Prediction cycle 2 | Likely non-defense function |
| pfam09919 | 98.4% | 0.11 | Prediction cycle 2 | Likely non-defense function |
| pfam16930 | 85.1% | 0.11 | Prediction cycle 2 | Likely non-defense function |
| pfam14113 | 71.7% | 0.14 | Prediction cycle 2 | Likely non-defense function |
| pfam13151 | 65.5% | 0.40 | Prediction cycle 2 | Possibly belongs to a new defense |
| | | | | system |
| pfam14328 | 71.3% | 0.13 | Prediction cycle 2 | Likely non-defense function |
| pfam07511 | 68.4% | 0.23 | Prediction cycle 2 | Likely mobilome |
| pfam14281 | 71.4% | 0.59 | Prediction cycle 2 | Possibly belongs to a new defense |
| | | | | system |
| pfam14584 | 79.8% | 0.12 | Prediction cycle 2 | Likely non-defense function |
| pfam11047 | 78.1% | 0.11 | Prediction cycle 2 | Likely non-defense function |
| pfam11660 | 70.4% | 0.20 | Prediction cycle 2 | Likely mobilome |
| pfam11190 | 67.1% | 0.20 | Prediction cycle 2 | Likely mobilome |
| pfam14103 | 97.7% | 0.39 | Prediction cycle 2 | Likely known system |
| pfam09686 | 68.6% | 0.20 | Prediction cycle 2 | Likely mobilome |
| pfam15978 | 75.8% | 0.25 | Prediction cycle 2 | Likely mobilome |
| pfam09376 | 67.6% | 0.33 | Prediction cycle 2 | Possibly belongs to a new defense |
| | | | | system |
| pfam12602 | 69.9% | 0.18 | Prediction cycle 2 | Likely mobilome |
| pfam07455 | 91.6% | 0.11 | Prediction cycle 2 | Likely mobilome |
| pfam16452 | 82.1% | 0.13 | Prediction cycle 2 | Likely mobilome |
| pfam11072 | 75.0% | 0.20 | Prediction cycle 2 | Likely mobilome |
| pfam14253 | 69.7% | 0.30 | Prediction cycle 2 | Known system |
| pfam15731 | 83.3% | 0.28 | Prediction cycle 2 | Known system |
| pfam05419 | 65.3% | 0.43 | Prediction cycle 2 | Possibly belongs to a new defense |
| | | | | system |
| pfam12870 | 65.4% | 0.18 | Prediction cycle 2 | Likely non-defense function |
| pfam10723 | 78.2% | 0.20 | Prediction cycle 2 | Likely mobilome |
| pfam14302 | 67.0% | 0.10 | Prediction cycle 2 | Likely non-defense function |
| pfam14348 | 69.7% | 0.23 | Prediction cycle 2 | Likely mobilome |
| pfam10049 | 75.1% | 0.51 | Prediction cycle 2 | Likely known system |
| pfam13814 | 65.5% | 0.25 | Prediction cycle 2 | Likely mobilome |
| pfam12834 | 67.9% | 0.27 | Prediction cycle 2 | Likely mobilome |
| pfam09937 | 66.2% | 0.10 | Prediction cycle 2 | Likely non-defense function |
| pfam08937 | 67.8% | 0.33 | Prediction cycle 2 | Possibly belongs to a new defense |
| | | | | system |
| pfam09903 | 65.6% | 0.26 | Prediction cycle 2 | Possibly belongs to a new defense |
| | | | | system |
| pfam02521 | 90.4% | 0.11 | Prediction cycle 2 | Likely non-defense function |
| pfam12835 | 70.7% | 0.31 | Prediction cycle 2 | Likely mobilome |
| pfam02387 | 75.9% | 0.20 | Prediction cycle 2 | Likely mobilome |
| pfam13784 | 65.1% | 0.47 | Prediction cycle 2 | Possibly belongs to a new defense |
| | | | | system |
| pfam09848 | 65.6% | 0.45 | Prediction cycle 2 | Known system |
| pfam12950 | 67.4% | 0.39 | Prediction cycle 2 | Known system |
| pfam13166 | 75.6% | 0.39 | Prediction cycle 2 | Known system |
| pfam13555 | 70.4% | 0.27 | Prediction cycle 2 | Possibly belongs to a new defense |
| | | | | system |
| pfam01927 | 65.2% | 0.19 | Prediction cycle 2 | Likely known system |
| pfam06006 | 93.4% | 0.14 | Prediction cycle 2 | Likely mobilome |
| pfam06174 | 98.7% | 0.10 | Prediction cycle 2 | Likely mobilome |
| pfam11398 | 85.9% | 0.10 | Prediction cycle 2 | Likely non-defense function |
| pfam12686 | 65.4% | 0.32 | Prediction cycle 2 | Likely mobilome |
| pfam03596 | 70.8% | 0.12 | Prediction cycle 2 | Likely non-defense function |
| pfam06527 | 68.3% | 0.31 | Prediction cycle 2 | Likely mobilome |
| pfam07799 | 70.7% | 0.12 | Prediction cycle 2 | Likely mobilome |
| pfam13470 | 67.0% | 0.43 | Prediction cycle 2 | Known system |
| pfam13588 | 68.3% | 0.37 | Prediction cycle 2 | Known system |
| pfam05713 | 65.0% | 0.39 | Prediction cycle 2 | Likely mobilome |
| pfam05272 | 68.0% | 0.17 | Prediction cycle 2 | Uncertain |
| pfam13749 | 69.0% | 0.43 | Prediction cycle 2 | Possibly belongs to a new defense |
| | | | | system |
| pfam13657 | 69.5% | 0.30 | Prediction cycle 2 | Known system |
| pfam04326 | 65.1% | 0.39 | Prediction cycle 2 | Possibly belongs to a new defense |
| | | | | system |
| pfam06769 | 96.5% | 0.12 | Prediction cycle 2 | Known system |
| pfam02498 | 66.8% | 0.17 | Prediction cycle 2 | Likely mobilome |
| pfam13173 | 68.3% | 0.26 | Prediction cycle 2 | Possibly belongs to a new defense |
| | | | | system |
| pfam05685 | 67.2% | 0.27 | Prediction cycle 2 | Uncertain |
| pfam13538 | 66.9% | 0.13 | Prediction cycle 2 | Possibly belongs to a new defense |
| | | | | system |
| pfam01402 | 67.6% | 0.12 | Prediction cycle 2 | Likely non-defense function |
| pfam12705 | 76.4% | 0.10 | Prediction cycle 2 | Likely non-defense function |
| pfam13175 | 73.9% | 0.37 | Prediction cycle 2 | Possibly belongs to a new defense |
| | | | | system |
| pfam13304 | 63.8% | 0.33 | Prediction cycle 2 | Possibly belongs to a new defense |
| | | | | system |

⁴Makarova et al. J Bacteriol. 2011 Nov; 193(21): 6039-6056

From genes to systems: Each of the putative defense-related gene families was used as an anchor to search for multi-gene systems, as follows. The protein coding sequences for neighboring genes (+/−10 genes) for all family members were clustered based on sequence homology (for example, if pfamY is found within 50 genomes in our set, the 20 neighboring genes in each genome, plus the pfamY gene in each genome, were taken—altogether 50*21=1050 genes to be clustered). Clustering was done with OrthoMCL software v2.0.9 (Li L, Stoeckert C J, Roos D S. (2003) OrthoMCL: identification of ortholog groups for eukaryotic genomes. Genome Res. 13: 2178-89) with blastp parameters [-F ′m S′-v 100000 -b 100000 -e 1e-5 -m 8] and with mcl v12.068 downloaded from http://micans.org/mcl/ (Enright A J, Van Dongen S, Ouzounis C A. (2002) An efficient algorithm for large-scale detection of protein families. nucleic Acids Res. 30: 1575-84; van Dongen S, Abreu-Goodger C. (2012) Using MCL to Extract Clusters from Networks. Methods in molecular biology (Clifton, N.J.). pp. 281-295) with inflation value of 1.1. When the number of blastp hits for a given anchor pfam was too large and prohibitive for OrthoMCL to generate clusters (>75 million blastp hits), a subset of genomes, containing only bacterial and archaeal genomes annotated as “complete” (rather than “draft”) was used for clustering.

To detect the most prevalent genes around the anchor pfam, only the 10% largest clusters (“frequent clusters”) were considered. For the sake of cluster size calculation, genes originating from the same species (derived from the strain name in the NCBI annotation) were counted as one gene, to prevent organisms for which many strains have been sequenced from inflating the cluster size. An edge between cluster(i) and cluster(j) was defined if a gene from cluster(j) followed a gene from cluster(i) in a given genome with no other genes belonging to frequent clusters found in between, with edge weight (“thickness”) defined as the number of such adjacency cases. Again, edge weights were adjusted such that multiple appearances of a cluster pair originating from the same species were recorded as a single appearance. Only the 10% thickest edges were retained for further analysis. In each genome, the maximal “path” that included the anchor pfam gene and was composed of the retained (largest) clusters and the retained (thickest) edges was recorded. Such a “path”, representing a set of genes appearing in a conserved order in multiple genomes, was considered a candidate multi-gene system. Infrequent variations on the gene order and composition of common systems were merged into the common system if they shared at least 50% of their clusters and had less than 25% appearances than the common system. Only systems with five or more appearances from different species were further analyzed.

The domains within the gene members of each system were analyzed bioinformatically using the tools HHpred (Soding J, Biegert A, Lupas A N. (2005) The HHpred interactive server for protein homology detection and structure prediction. nucleic Acids Res. 33: W244-W248; Alva V, Nam S-Z, Soding J, Lupas A N, I. S, S. C, et al. (2016) The MPI bioinformatics Toolkit as an integrative platform for advanced protein sequence and structure analysis. nucleic Acids Res. Oxford University Press; 44: W410-W415), Phyre2 (Kelley L A, Mezulis S, Yates C M, Wass M N, Sternberg M J E. (2015) The Phyre2 web portal for protein modeling, prediction and analysis. Nat Protoc. Nature Research; 10: 845-858), PSI-BLAST (Altschul S F, Madden T L, Schïffer A A, Zhang J, Zhang Z, Miller W, et al. (1997) Gapped BLAST and PSI-BLAST: a new generation of protein database search programs. nucleic Acids Res. 25: 3389-402), and NCBI's Conserved Domain Database (CDD) (Marchler-Bauer A, Lu S, Anderson J B, Chitsaz F, Derbyshire M K, DeWeese-Scott C, et al. (2011) CDD: a Conserved Domain Database for the functional annotation of proteins. nucleic Acids Res. 39: D225-D229). The systems were then manually filtered, based on this analysis, to remove (i) known defense systems whose domains did not appear in our initial set of gene families known to participate in defense; (ii) systems likely representing mobile genetic elements (“mobilome”) and; (iii) systems likely participating in non-defensive functions or house-keeping systems (Table 2).

A second cycle of prediction was then performed, expanding the set of “positive” gene families from Table 1 to include the gene families participating in the candidate new defense systems, as well as the gene families participating in known defense systems that were previously missing from our set and detected in the first round. All pfams were again scanned and the same thresholds were applied (defense score 65%, context variability score 0.1). New pfams retrieved from the second cycle were analyzed as above to generate and annotate multi-gene systems.

Candidate new systems were further prioritized to select instances for experimental validations. Systems tagged as “questionable”, due to uncertainty whether they represent defense genes or mobile genetic elements, were filtered out (Table 3). Systems existing in only a narrow range of organisms, as well as systems that were not found in organisms phylogenetically close either to E. coli or B. subtilis, were not tested experimentally (Table 3).

TABLE 3
Candidate Defense Systems

| | | Included/ | |
|---|---|---|---|
| | | Excluded from | |
| Anchor protein | Number | experimental | |
| families | of genes? | study | Reason for exclusion |
| pfam04033 | 1 | Excluded | Archaea specific |
| pfam11536 | 2 | Excluded | Thermococcus specific |
| pfam12635; | 3-4 | Included | — |
| pfam06634; | | | |
| COG1483 | | | |
| pfam09544 | 3-4 | Excluded | Myxococcales specific |
| pfam15611; | 3-4 | Included | — |
| COG0553 | | | |
| pfam13910 | 1 | Included | — |
| pfam14130 | 2 | Included | — |
| pfam14335 | 1-2 | Excluded | No locus that is a stand- |
| | | | alone gene, always |
| | | | within R-M systems |
| pfam12476; | 3 | Included | — |
| pfam03235 | | | |
| pfam08878 | 2 | included | — |
| pfam13208; | 3 | Included | — |
| pfam15615; | | | |
| pfam10923 | | | |
| pfam10980 | 7 | Included | — |
| pfam08814; | 2 | Excluded | Cyanobacteria specific |
| pfam08869 | | | |
| pfam14267 | 1 | Included | — |
| pfam06067 | 1-2 | Excluded | Loci in Bacillus are in |
| | | | phage/mobilome |
| | | | context |
| pfam13635; | 1 | Included | — |
| pfam13173 | | | |
| pfam13604; | 1 | Included | — |
| pfam13538 | | | |
| pfam06709 | 1 | Excluded | Vibrio specific |
| pfam07154 | 1 | Excluded | Cyanobacteria specific |
| pfam11994 | 3 | Excluded | Not found in |
| | | | bacillus/coli |
| COG1205 | 2-5 | Included | — |
| COG4913; | 3-4 | Included | — |
| COG4924; | | | |
| pfam11796; | | | |
| pfam09664; | | | |
| pfam13555 | | | |
| pfam04383 | 1 | Included | — |
| COG3586 | 1 | Included | — |
| COG3950 | 2 | Included | — |
| COG4804 | 1 | Included | — |
| COG1451 | 1 | Included | — |
| pfam09954 | 1 | Excluded | No appropriate locus |
| | | | that is a stand-alone |
| | | | gene, always comes |
| | | | with other genes + |
| | | | already exists in |
| | | | BEST7003 |
| pfam16416; | 2-4 | Excluded | Cyanobacteria specific |
| pfam05419 | | | |
| pfam04485 | 4 | Excluded | Cyanobacteria specific |
| pfam16162 | 2 | Included | — |
| pfam16263 | 1 | Excluded | Loci in Bacillus are in |
| | | | phage/mobilome |
| | | | context |
| pfam12668; | 2 | Included | — |
| pfam13151 | | | |
| pfam14082 | 1 | Included | — |
| pfam14281 | 1 | Included | — |
| pfam09376 | 3 | Included | — |
| pfam08937 | 2 | Included | — |
| pfam09903 | 1 | Included | — |
| pfam13784 | 1 | Included | — |
| pfam13749; | 1 | Included | — |
| pfam04326 | | | |
| pfam13175; | 2 | included | — |
| pfam13304 | | | |

Phylogenetic Distribution Analysis of New Systems:

For each validated defense system, several loci including the locus that was experimentally verified were taken as seeds for psi-Blast. psi-Blast version 2.5.0 of BLAST+ (Altschul S F, Madden T L, Schïffer A A, Zhang J, Zhang Z, Miller W, Lipman D J. (1997) Gapped BLAST and PSI-BLAST: a new generation of protein database search programs. nucleic Acids Res.; 25: 3389-402; Camacho C, Coulouris G, Avagyan V, Ma N, Papadopoulos J, Bealer K, Madden TL. (2009) BLAST+: architecture and applications. BMC Bioinformatics. 10: 421. doi: 10.1186/1471 -2105-10-421) with parameters [-num_iterations 10 -max_hsps 1 -max_target_seqs 100000 -evalue 1e-10] was performed for each protein of each system, against all microbial genomes downloaded from NCBI on April 2016. When the hits of all proteins of a system were found closely localized on a genome, spanning no more than 150% of the length of the original system, this genome was recorded as containing the system. For the Druantia and Wadjet systems, -evalue 1e-5 was used to enable detection of distant homologs. For systems with 4 or 5 genes (Zorya type I, Druantia types I and II, Wadjet), systems were reported if at least 3 of their genes were identified. For the Druantia system, systems with hits to the DruE protein were retained if the DruE size was >1300aa. For the Thoeris system, multiple thsB genes near the thsA gene were recorded if they were within 10 kb of genomic DNA around the identified thsA. Phylum for each genome was obtained using the JGI taxonomy server (https://taxonomyjgi-psf.org/).

Experimental Validation of Defense Systems

Cloning of the Systems into E. coli MG1655 and/or B. subtilis BEST7003

A cloning shuttle vector for large fragments was constructed as previously described (Ofir G, Melamed S, Sberro H, Silverman S, Doron S, Yaakov G, Mukamel Z, Sorek R. (2017) DISARM, a novel bacterial defense system with broad anti-phage activities, submitted). The vector contains a p15a origin of replication and ampicillin resistance for plasmid propagation in E. coli, and amyE integration cassette with spectinomycin resistance for genomic integration into B. subtilis. The backbone of this vector was amplified using primers OGO309+OGO310, adding to it a BamHI restriction site and a terminator site upstream to the insert cloning site. The multiple cloning site of plasmid pBS1C (Radeck J, Kraft K, Bartels J, Cikovic T, Dürr F, Emenegger J, Kelterborn S, Sauer C, Fritz G, Gebhard S, Mascher T. (2013) The Bacillus BioBrick Box: generation and evaluation of essential genetic building blocks for standardized work with Bacillus subtilis. J Biol Eng. BioMed Central; 7: 29. doi:10.1186/1754-1611-7-29), received from BGSC (accession ECE257), was amplified using primers OGO311+OGO312. Both fragments were digested using AscI and BamHI, ligated using T4 ligase and transformed into E. coli, resulting in plasmid pSG1-rfp.

The loci of most systems were commercially synthesized by Genscript and cloned, also by Genscript, directly into pSG1-rfp between the AscI and NotI sites of the multiple cloning site (Table 4: “Cloning method” column).

TABLE 4
Loci for Candidate Systems

| Anchor | | Construct | Donor bacteria | | |
|---|---|---|---|---|---|
| protein | System | number | (IMG | Position | Length |
| families | name | (C.N.) | accession) | (strand) | in bp |
| pfam16162 | Kiwa | 1 | Bacillus sp. | 1801073-1803310 | 2238 |
| | | | FJAT-27997 | (+) | |
| | | | (Ga0081961_11) | | |
| | Kiwa | 2 | E. coli O55:H7 | 5120652-5122819 | 2168 |
| | | | RM12579 | (−) | |
| | | | (CP003109) | | |
| pfam08878 | Hachiman | 3 | B. cereus | 106560-111167 | 5122 |
| | | | B4087 | (−) | |
| | | | (phylogroup | | |
| | | | III-12 9) | | |
| | | | (LCYM01000053) | | |
| | Hachiman | 4 | B. cereus | 77887-82731 | 4845 |
| | | | H3081.97 | (+) | |
| | | | (ABDL02000007) | | |
| pfam13749; | | 5 | B. iranensis | 391132-393430 | 2299 |
| pfam04326 | | | DSM 23995 | (+) | |
| | | | (Ga0075261_103) | | |
| | | 6 | B. boroniphilus | 3453-5851 | 2399 |
| | | | JCM 21738 | (−) | |
| | | | (Ga0054992_1085) | | |
| pfam13175; | Gabija | 7 | B. cereus | 172515-177839 | 5325 |
| pfam13304 | | | HuB5-5 | (+) | |
| | | | (AHEG01000016) | | |
| | Gabija | 8 | B. cereus | 93871-97763 | 3893 |
| | | | VD045 | (+) | |
| | | | (AHET01000033) | | |
| pfam13208; | | 9 | B. methanolicus | 1037084-1043201 | 6118 |
| pfam15615; | | | PB1 | (−) | |
| pfam10923 | | | (AFEU01000001) | | |
| | | 10 | B. cereus | 118058-124729 | 6672 |
| | | | NVH0597-99 | (−) | |
| | | | ABDK02000013 | | |
| | | | (ABDK02000013) | | |
| pfam04383 | | 11 | Enterococcus | 142827-143797 | 970 |
| | | | faecalis TR197 | (+) | |
| | | | (ASDV01000010) | | |
| | | 12 | Lactobacillus | 785262-786580 | 1320 |
| | | | plantarum | (−) | |
| | | | subsp. | | |
| | | | plantarum ST- | | |
| | | | III chromosome | | |
| | | | (NC_014554) | | |
| pfam14130 | Lamassu | 13 | Bacillus sp. | 104996-108335 | 3358 |
| | | | NIO-1130 | (−) | |
| | | | (Ga0061086_109) | | |
| | Lamassu | 14 | B. cereus | 52399-56035 | 3637 |
| | | | VD014 | (+) | |
| | | | (AHER01000061) | | |
| pfam10980 | | 15 | Pseudoalteromonas | 119710-124196 | 4486 |
| | | | marina | (−) | |
| | | | mano4 | | |
| | | | (AHCB02000060) | | |
| | | 16 | E. coli UMEA | 116829-122750 | 5921 |
| | | | 3355-1 | (−) | |
| | | | (AWDK01000015) | | |
| pfam13604; | | 17 | B. cereus | 120797-124381 | 3585 |
| pfam13538 | | | HuA2-1 | (+) | |
| | | | (AHDV01000009) | | |
| | | 18 | B. paralicheniformis | 4324128-4327523 | 3382 |
| | | | ATCC 9945a | (−) | |
| | | | (CP005965 | | |
| | | | (NC 021362)) | | |
| pfam13635; | | 19 | Bacillus sp. | 420-2978 | 2559 |
| pfam13173 | | | L1(2012) | (−) | |
| | | | (BLTDRAFT_ANNK01000002_1.2) | | |
| | | 20 | Bacillus sp. | 98213-100268 | 2056 |
| | | | MT2 | (+) | |
| | | | (Ga0058649_116) | | |
| pfam12668; | | 21 | B. cereus | 4327581-4328881 | 1301 |
| pfam13151 | | | G9241 | (−) | |
| | | | (Ga0077877_11) | | |
| | | 22 | B. cereus | 92524-94334 | 1811 |
| | | | R309803 | (−) | |
| | | | (NZ_ACLY01000076) | | |
| pfam09376 | | 23 | B. cereus Q1 | 1003565-1008191 | 4627 |
| | | | (NC_011969) | (+) | |
| | | 24 | B. subtilis Hall | 25333-30937 | 5605 |
| | | | (A921DRAFT_AMCA01000155_1.155) | (−) | |
| pfam13784 | | 25 | E. coli H397 | 928-2724 | 1797 |
| | | | (AEXH01000043) | (−) | |
| | | 26 | E. coli UMEA | 254125-256825 | 2000 |
| | | | 3108-1 | (+) | |
| | | | (AWBD01000001) | | |
| pfam14082 | Shedu | 27 | B. cereus | 186286-187555 | 1270 |
| | | | BAG2O-2 | (−) | |
| | | | (AHCX01000044) | | |
| | Shedu | 28 | B. cereus | 954837-957049 | 2213 |
| | | | B4264 | (−) | |
| | | | (NC_011725) | | |
| pfam12635; | | 29 | Paenibacillus | 743619-754251 | 10633 |
| pfam06634; | | | sabinae T27 | (+) | |
| COG1483 | | | (CP004078) | | |
| COG4804 | | 30 | Paenibacillus | 649-3948 | 3300 |
| | | | sophorae S27 | (+) | |
| | | | (L689DRAFT_ASSF01000040_1.18) | | |
| pfam08937 | Thoeris | 31 | B. cereus MSX- | 16453-19685 | 2477 |
| | | | D12 | (+) | |
| | | | (AHEQ01000050) | | |
| | Thoeris | 32 | B. amyloliquefaciens | 2071378-2073427 | 2050 |
| | | | Y2 | (−) | |
| | | | (CP003332) | | |
| | Thoeris | 33 | B. cereus | 93628-95745 | 1689 |
| | | | HuB4-4 | (+) | |
| | | | (AHEF01000014) | | |
| | Thoeris | 34 | B. thuringiensis | 134769-136361 | 1590 |
| | | | serovar | (−) | |
| | | | finitimus YBT- | | |
| | | | 020 plasmid | | |
| | | | pBMB26 | | |
| | | | (CP002509) | | |
| pfam14267 | | 35 | Paenibacillus | 94753-96069 | 1316 |
| | | | macerans 8244 | (−) | |
| | | | (Ga0097770_129) | | |
| | | 36 | B. cereus | 57053-58320 | 1154 |
| | | | BAG2X1-1 | (−) | |
| | | | (AHCZ01000031) | | |
| pfam12476; | | 37 | Solibacillus | 1395361-1399023 | 3663 |
| pfam03235 | | | silvestris | (+) | |
| | | | (AP012157) | | |
| | | 38 | Tumebacillus | 100864-104082 | 3219 |
| | | | flagellatus | (−) | |
| | | | GST4 | | |
| | | | (JMIR01000008) | | |
| | | 39 | Paenibacillus | 23305-26493 | 3189 |
| | | | sp. FSL R7-269 | (+) | |
| | | | (ASPS01000025) | | |
| COG1451 | | 40 | B. cereus | 119987-121875 | 1888 |
| | | | MC67 | (+) | |
| | | | (AHEN01000008) | | |
| | | 41 | B. cereus IS195 | 240659-242023 | 1364 |
| | | | (AHEH01000058) | (+) | |
| COG3950 | Septu | 42 | B. thuringiensis | 2740820-2743903 | 3083 |
| | | | HD12 | (+) | |
| | | | (Ga0125139_15) | | |
| | Septu | 43 | B. weihenstephanensis | 3499862-3504110 | 3745 |
| | | | KBAB4 | (−) | |
| | | | (NC_010184) | | |
| | Septu | 44 | B. cereus | 90978-93642 | 2665 |
| | | | m1293 | (−) | |
| | | | (NZ_ACLS01000104) | | |
| COG3586 | | 45 | B. paralicheniformis | 829842-834035 | 4194 |
| | | | ATCC 9945a | (+) | |
| | | | (CP005965 | | |
| | | | (NC_021362)) | | |
| | | 46 | B. vallismortis | 121062-122280 | 1219 |
| | | | DV1-F-3 | (+) | |
| | | | (GYSDRAFT_AFSH01000055_1.55) | | |
| | | 47 | Bacillus sp. | 1839870-1841334 | 1465 |
| | | | BC15 | (+) | |
| | | | (Ga0066720_10) | | |
| pfam09903 | | 48 | Geobacter sulfurreducens | 72105-73998 | 1894 |
| | | | AM-1 | (+) | |
| | | | (Ga0098194_11) | | |
| pfam15611; | Zorya | 49 | E. coli | 298890-307639 | 8750 |
| COG0553 | | | E24377A | (−) | |
| | | | (NC_009801) | | |
| | Zorya | 50 | E. coli KOEGE | 522466-531355 | 8890 |
| | | | 71 (186a) | (+) | |
| | | | (AWAO01000003) | | |
| | Zorya | 51 | E. coli ATCC | 4265695-4269611 | 3917 |
| | | | 8739 | (+) | |
| | | | (NC_010468) | | |
| pfam14281 | | 52 | B. cereus LK9 | 37579-39692 | 2114 |
| | | | (Ga0081098-108) | (−) | |
| | | 53 | B. cereus | 14516-15854 | 1339 |
| | | | VD200 | (+) | |
| | | | (AHFM01000019) | | |
| | | 54 | B. subtilis HM- | 187758-189194 | 1437 |
| | | | 66 | (−) | |
| | | | (Ga0124450_104) | | |
| COG1205 | Druantia | 55 | E. coli KTE181 | 35990-46193 | 10204 |
| | | | (ANTC01000054) | (+) | |
| | Druantia | 56 | E. coli UMEA | 219529-231495 | 11967 |
| | | | 4076-1 | (+) | |
| | | | (AWEM01000032) | | |
| | Druantia | 57 | B. cereus LK9 | 4022-14331 | 10310 |
| | | | (Ga0081098_108) | (−) | |
| pfam13910 | | 58 | B. megaterium | 3517210-3519233 | 2024 |
| | | | Q3 | (−) | |
| | | | (Ga0081752_11) | | |
| COG4913; | Wadjet | 59 | B. cereus Q1 | 1011280-1018329 | 7050 |
| COG4924; | | | (NC_011969) | (+) | |
| pfam11796; | | | | | |
| pfam09664; | | | | | |
| pfam13555 | | | | | |
| | Wadjet | 60 | B. vireti LMG | 67917-76250 | 8334 |
| | | | 21834 | (−) | |
| | | | (ALAN01000059) | | |
| | Wadjet | 61 | B. thuringiensis | 1018412-1025427 | 7013 |
| | | | serovar | (+) | |
| | | | finitimus YBT- | | |
| | | | 020 plasmid | | |
| | | | pBMB26 | | |
| | | | (CP002508) | | |
| Controls | | | | | |
| Type III | | 63 | Lactobacillus | 1034479-1039919 | 5441 |
| RM system | | | rhamnosus GG | (+) | |
| (BceSI) | | | (AP011548) | | |
| Type III | | 64 | B. thuringiensis | 52683-53616 | 934 |
| TA system | | | sv. kurstaki | (+) | |
| (ToxIN) | | | HD-1 | | |
| | | | (CP004874) | | |
| Type II RM | | 65 | B. amyloliquefaciens | 3187589-3190876 | 3192 |
| system | | | DSM 7 | (+) | |
| (BamHI) | | | (NC_014551) | | |
| Abi system | | 66 | B. cereus | 18460-19683 | 1224 |
| (AbiH) | | | ATCC 10876 | (−) | |
| | | | (NZ_ACLT01000157) | | |
| Type I RM | | 67 | B. glycinifermentans | 59672-66665 | 6994 |
| system | | | TH008 | (−) | |
| (BliDi) | | | (Ga0077513_1025) | | |
| DISARM | | 68 | B. paralicheniformis | 815730-826377 | 10648 |
| | | | ATCC 9945a | (+) | |
| | | | (CP005965 | | |
| | | | (NC_021362)) | | |
| Negative | | 69 | (empty vector) | | 0 |
| Control | | | | | |

| | | | | | NA | | Open |
|---|---|---|---|---|---|---|---|
| Anchor | | Construct | | | SEQ | | Reading |
| protein | System | number | Cloning | Cloning | ID | | Frame |
| families | name | (C.N.) | method | successful? | NO: | Expressed? | Coordinates |
| pfam16162 | Kiwa | 1 | Synthesis | yes | | yes | |
| | Kiwa | 2 | Synthesis | yes | SEQ | yes | kwaA: 572-1159 |
| | | | | | ID | | kwaB: 1156-2103 |
| | | | | | NO: 3 | | |
| pfam08878 | Hachiman | 3 | Synthesis | yes | SEQ | yes | hamA: 339-1154 |
| | | | | | ID | | hamB: 1114-4506 |
| | | | | | NO: 4 | | |
| | Hachiman | 4 | Synthesis | yes | | yes | |
| pfam13749; | | 5 | Synthesis | yes | | yes | |
| pfam04326 | | | | | | | |
| | | 6 | Synthesis | yes | | yes | |
| pfam13175; | Gabija | 7 | Synthesis | yes | SEQ | yes | gajA: 810-2870 |
| pfam13304 | | | | | ID | | gajB: 2873-4927 |
| | | | | | NO: 5 | | |
| | Gabija | 8 | Synthesis | yes | SEQ | yes | gajA: 320-2056 |
| | | | | | ID | | gajB: 2058-3542 |
| | | | | | NO: 6 | | |
| pfam13208; | | 9 | Synthesis | yes | | yes | |
| pfam15615; | | | | | | | |
| pfam10923 | | | | | | | |
| | | 10 | Synthesis | yes | | yes | |
| pfam04383 | | 11 | Synthesis | yes | | yes | |
| | | 12 | Synthesis | yes | | yes | |
| pfam14130 | Lamassu | 13 | Strain | yes | SEQ | yes | lmuA: 361-1308 |
| | | | obtained | | ID | | lmuB: 1314-3002 |
| | | | from | | NO: 7 | | |
| | | | DSMZ | | | | |
| | | | (DSM | | | | |
| | | | 25149) | | | | |
| | Lamassu | 14 | Synthesis | yes | SEQ | yes | lmuA: 877-1863 |
| | | | | | ID | | lmuB: 1868-3562 |
| | | | | | NO: 8 | | |
| pfam10980 | | 15 | Synthesis | yes | | yes | |
| | | 16 | Synthesis | no | | — | |
| pfam13604; | | 17 | Synthesis | yes | | yes | |
| pfam13538 | | | | | | | |
| | | 18 | Strain | yes | | yes | |
| | | | obtained | | | | |
| | | | from | | | | |
| | | | ATCC | | | | |
| | | | (9945a) | | | | |
| pfam13635; | | 19 | Synthesis | yes | | yes | |
| pfam13173 | | | | | | | |
| | | 20 | Synthesis | yes | | yes | |
| pfam12668; | | 21 | Synthesis | yes | | yes | |
| pfam13151 | | | | | | | |
| | | 22 | Synthesis | yes | | yes | |
| pfam09376 | | 23 | Synthesis | yes | | yes | |
| | | 24 | Synthesis | yes | | yes | |
| pfam13784 | | 25 | Synthesis | yes | | yes | |
| | | 26 | Synthesis | yes | | yes | |
| pfam14082 | Shedu | 27 | Synthesis | yes | | yes | |
| | Shedu | 28 | Synthesis | yes | SEQ | yes | sduA: 760-1902 |
| | | | | | ID | | |
| | | | | | NO: 9 | | |
| pfam12635; | | 29 | Strain | no | | — | |
| pfam06634; | | | obtained | | | | |
| COG1483 | | | from | | | | |
| | | | DSMZ | | | | |
| | | | (DSM | | | | |
| | | | 17841) | | | | |
| COG4804 | | 30 | Synthesis | yes | | yes | |
| pfam08937 | Thoeris | 31 | Synthesis | yes | SEQ | yes | thsA: 205-1635 |
| | | | | | ID | | thsB: 1674-2252 |
| | | | | | NO: | | |
| | | | | | 10 | | |
| | Thoeris | 32 | Synthesis | yes | SEQ | yes | thsA: 298-1191 |
| | | | | | ID | | thsB: 1181-1753 |
| | | | | | NO: | | |
| | | | | | 11 | | |
| | Thoeris | 33 | Synthesis | yes | | yes | |
| | Thoeris | 34 | Strain | yes | | no | |
| | | | obtained | | | | |
| | | | from BGSC | | | | |
| | | | (4B4) | | | | |
| pfam14267 | | 35 | Synthesis | yes | | no | |
| | | 36 | Synthesis | yes | | no | |
| pfam12476; | | 37 | Synthesis | yes | | yes | |
| pfam03235 | | | | | | | |
| | | 38 | Synthesis | no | | — | |
| | | 39 | Synthesis | no | | — | |
| COG1451 | | 40 | Synthesis | yes | | yes | |
| | | 41 | Synthesis | yes | | yes | |
| COG3950 | Septu | 42 | Strain | yes | SEQ | yes | ptuA: 434-1624 |
| | | | obtained | | ID | | ptuB: 1621-2394 |
| | | | from BGSC | | NO: | | |
| | | | (4K1) | | 12 | | |
| | Septu | 43 | Synthesis | yes | SEQ | yes | ptuA: 251-2380 |
| | | | | | ID | | ptuB: 2389-3276 |
| | | | | | NO: | | |
| | | | | | 13 | | |
| | Septu | 44 | Strain | no | | — | |
| | | | obtained | | | | |
| | | | from BGSC | | | | |
| | | | (6A54) | | | | |
| COG3586 | | 45 | Strain | yes | | yes | |
| | | | obtained | | | | |
| | | | from | | | | |
| | | | ATCC | | | | |
| | | | (9945a) | | | | |
| | | 46 | Strain | yes | | yes | |
| | | | obtained | | | | |
| | | | from BGSC | | | | |
| | | | (28A4) | | | | |
| | | 47 | Synthesis | yes | | yes | |
| pfam09903 | | 48 | Synthesis | yes | | yes | |
| pfam15611; | Zorya | 49 | Synthesis | yes | SEQ | yes | zorA: 364-2553 |
| COG0553 | | | | | ID | | zorB: 2567-3307 |
| | | | | | NO: | | zorC: 3304-4986 |
| | | | | | 14 | | zorD: 4986-8228 |
| | Zorya | 50 | Synthesis | yes | | yes | |
| | Zorya | 51 | Strain | yes | SEQ | yes | zorA: 213-1865 |
| | | | obtained | | ID | | zorB: 1872-2579 |
| | | | from | | NO: | | zorE: 2583-3686 |
| | | | ATCC | | 15 | | |
| | | | (8739) | | | | |
| pfam14281 | | 52 | Synthesis | yes | | yes | |
| | | 53 | Synthesis | yes | | yes | |
| | | 54 | Synthesis | yes | | no | |
| COG1205 | Druantia | 55 | Strain | yes | | yes | |
| | | | obtained | | | | |
| | | | from BEI | | | | |
| | | | Resources | | | | |
| | | | (NR- | | | | |
| | | | 32771) | | | | |
| | Druantia | 56 | Synthesis | yes | SEQ | yes | druA: 378-1592 |
| | | | (fragments) | | ID | | druB: 1614-3260 |
| | | | | | NO: | | druC: 3272-5155 |
| | | | | | 16 | | druD: 5167-6210 |
| | | | | | | | druE: 6207-11717 |
| | Druantia | 57 | Synthesis | yes | | yes | |
| | | | (fragments) | | | | |
| pfam13910 | | 58 | Synthesis | yes | | yes | |
| COG4913; | Wadjet | 59 | Synthesis | yes | SEQ | yes | jetA: 146-1552 |
| COG4924; | | | (fragments), | | ID | | jetB: 1562-2155 |
| pfam11796; | | | by Gen9 | | NO: | | jetC: 2133-5447 |
| pfam09664; | | | | | 17 | | jetD: 5551-6636 |
| pfam13555 | | | | | | | |
| | Wadjet | 60 | Synthesis | yes | SEQ | yes | jetAⁱⁱ: 230-1717 |
| | | | | | ID | | jetBⁱⁱ: 1717-2883 |
| | | | | | NO: | | jetCⁱⁱ: 2883-6992 |
| | | | | | 18 | | jetDⁱⁱ: 6985-8280 |
| | Wadjet | 61 | Strain | yes | SEQ | yes | jetDⁱⁱⁱ: 444-1700 |
| | | | obtained | | ID | | jetAⁱⁱⁱ: 1676-3070 |
| | | | from BGSC | | NO: | | jetBⁱⁱⁱ: 3030-3713 |
| | | | (4B4) | | 19 | | jetCⁱⁱⁱ: 3710-6931 |
| Controls | | | | yes | | yes | |
| Type III | | 63 | Strain | yes | | yes | |
| RM system | | | obtained | | | | |
| (BceSI) | | | from | | | | |
| | | | ATCC | | | | |
| | | | (53103) | | | | |
| Type III | | 64 | Strain | yes | | yes | |
| TA system | | | obtained | | | | |
| (ToxIN) | | | from BGSC | | | | |
| | | | (4D1) | | | | |
| Type II RM | | 65 | Strain | yes | | yes | |
| system | | | obtained | | | | |
| (BamHI) | | | from | | | | |
| | | | DSMZ | | | | |
| | | | (DSM 7) | | | | |
| Abi system | | 66 | Strain | yes | | yes | |
| (AbiH) | | | obtained | | | | |
| | | | from | | | | |
| | | | DSMZ | | | | |
| | | | (DSM | | | | |
| | | | 9378) | | | | |
| Type I RM | | 67 | Strain | yes | | yes | |
| system | | | obtained | | | | |
| (BliDi) | | | from BGSC | | | | |
| | | | (51A1) | | | | |
| DISARM | | 68 | Ofir et al. | yes | | yes | |
| | | | 2017 | | | | |
| Negative | | 69 | | yes | | yes | |
| Control | | | | | | | |

In one case (the Type I Wadjet system) the DNA was synthesized by Gen9 (Boston, Mass.) with synonymous modifications to optimize GC content. In case the donor strains were readily available the system was not synthesized but instead was directly amplified from the genomic DNA of the donor strain using KAPA HiFi HotStart ReadyMix (Kapa Biosystems KK2601) with primers as detailed in Table 5.

TABLE 5
Primers

| | | | |
|---|---|---|---|
| SEQ ID | | | |
| NO | Primer | Sequence (5′-3′) | Description |

pSG1

| | | | |
|---|---|---|---|
| SEQ ID | OGO309 | aaaGGATCCCTCTAGAGTTAACAAGAGTT | Amplification of plasmid backbone containing amyE |
| NO: 20 | | TGTAG | integration cassette with SpecR selection in B. subtilis, and |
| | | | p15a ori with ampR selection for E. coli. With OGO310 |
| SEQ ID | OGO310 | aaaGGCGCGCCATAAAAAACGCCCGGCG | Amplification of plasmid backbone containing amyE |
| NO: 21 | | GCAACCGAGCGTTCTGAATCGATCAGA | integration cassette with SpecR selection in B. subtilis, and |
| | | CCAGTTTTTAATTTG | p15a on with ampR selection for E. coli, adding |
| | | | terminator and AscI site upstream to the multiple cloning |
| | | | site. With OGO309 |
| SEQ ID | OGO311 | CTGATCGGATCCTAGAAG | Amplification of the biobrick MCS from pBSC1, use with |
| NO: 22 | | | OGO312 |
| SEQ ID | OGO312 | aaaGGCGCGCCCCCTTTTTTGCCGG | Amplification of the biobrick MCS from pBSC1, adding a |
| NO: 23 | | | AscI site, use with OGO311 |
| SEQ ID | OSM315 | GGCGCGCCATAAAAAAC | pSG1 backbone used in NEBuilder reactions, use with |
| NO: 24 | | | OSM316 |
| SEQ ID | OSM316 | GCGGCCGCGAATTC | |
| NO: 25 | | | |

Primers for putative systems

| | | | |
|---|---|---|---|
| SEQ ID | OSM319 | GTTGCCGCCGGGCGTTTTTTATGGCGCG | C.N. 42 to lift from strain, use with OSM320 |
| NO: 26 | | CCGGGAATGCAAAAAGGAGG | |
| SEQ ID | OSM320 | ATCCTAGAAGCTTATCGAATTCGCGGCC | |
| NO: 27 | | GCATATCATCCCTCGTTTTCGTC | |
| SEQ ID | OSM321 | ATCCTAGAAGCTTATCGAATTCGCGGCC | C.N. 44 to lift from strain, use with OSM322 |
| NO: 28 | | GCAGTGGATACAAAGAGTGAACG | |
| SEQ ID | OSM322 | GTTGCCGCCGGGCGTTTTTTATGGCGCG | |
| NO: 29 | | CCATTCATTCTTATTCCGCTAACTTTG | |
| SEQ ID | OSM325 | ATCCTAGAAGCTTATCGAATTCGCGGCC | C.N. 45 to lift from strain, use with OSM326 |
| NO: 30 | | GCGAGTTTGATCAAAGATTTGCC | |
| SEQ ID | OSM326 | GTTGCCGCCGGGCGTTTTTTATGGCGCG | |
| NO: 31 | | CCATTTAATTCTGCTTATCTCCAAG | |
| SEQ ID | OSM327 | ATCCTAGAAGCTTATCGAATTCGCGGCC | C.N. 13 to lift from strain, use with OSM328 |
| NO: 32 | | GCTGCATATTTATAATTGTGTGTTGAG | |
| SEQ ID | OSM328 | GTTGCCGCCGGGCGTTTTTTATGGCGCG | |
| NO: 33 | | CCAGAATGGAAAGTTGAATCAACTC | |
| SEQ ID | OSM333 | ATCCTAGAAGCTTATCGAATTCGCGGCC | C.N. 65 to lift from strain, use with OSM334 |
| NO: 34 | | GCACGATAAACTCGGTGTATTGAC | |
| SEQ ID | OSM334 | GTTGCCGCCGGGCGTTTTTTATGGCGCG | |
| NO: 35 | | CCAAAAGCTTTCGGCATCAAAG | |
| SEQ ID | OSM323 | ATCCTAGAAGCTTATCGAATTCGCGGCC | C.N. 46 to lift from strain, use with OSM324 |
| NO: 36 | | GCTGACCATTTCCCTTCTTTTTTAC | |
| SEQ ID | OSM324 | GTTGCCGCCGGGCGTTTTTTATGGCGCG | |
| NO: 37 | | CCTATAACCAACTCCCAATATATGG | |
| SEQ ID | OSM331 | ATCCTAGAAGCTTATCGAATTCGCGGCC | C.N. 67 to lift from strain, use with OSM332 |
| NO: 38 | | GCAACTAATAGTTTAAAACTTTACGGAT | |
| | | G | |
| SEQ ID | OSM332 | GTTGCCGCCGGGCGTTTTTTATGGCGCG | |
| NO: 39 | | CCTATCAAGCCCCCTCATTTCA | |
| SEQ ID | OSM340 | atcctagaagcttatcgaattcgcggccgcTTCGTTACTT | C.N. 29 to lift from strain, use with OSM341 |
| NO: 40 | | CTGGGATGGG | |
| SEQ ID | OSM341 | gttgccgccgggcgttttttatggcgcgccTGAAAAAGA | |
| NO: 41 | | CTCCTCCGTATTTG | |
| SEQ ID | OSM365 | atcctagaagcttatcgaattcgcggccgcTAACTGGAC | C.N. 34 to lift from strain, use with OSM366 |
| NO: 42 | | AGTGCTCTTGG | |
| SEQ ID | OSM366 | gttgccgccgggcgttttttatggcgcgccATGCCGATA | |
| NO: 43 | | GAAGATATTGCG | |
| SEQ ID | OSM342 | atcctagaagcttatcgaattcgcggccgcTTCCGATAC | C.N. 61 to lift from strain, use with OSM343a |
| NO: 44 | | TCTTTAGCCATAA | |
| SEQ ID | OSM343a | gttgccgccgggcgttttttatggcgcgccACAATCACA | |
| NO: 45 | | TCCTTATTTTCTT | |
| SEQ ID | OSM343b | atcctagaagcttatcgaattcgcggccgcATCTTTTTGA | C.N. 18 to lift from strain, use with OSM344 |
| NO: 46 | | ATAGTTTCAATGATG | |
| SEQ ID | OSM344 | gttgccgccgggcgttttttatggcgcgccAGTGTGGTA | |
| NO: 47 | | TGGAGATTAGC | |
| SEQ ID | OSM359 | atcctagaagcttatcgaattcgcggccgcGTGCTTAGA | C.N. 57 to lift fragment 1 from Genscript plasmid, use |
| NO: 48 | | TACGAAGGG | with OSM360 |
| SEQ ID | OSM360 | TAGCTCATTCTCTACAAAATCC | |
| NO: 49 | | | |
| SEQ ID | OSM361 | TCGATAGAGGATTTTGTAGAG | C.N. 57 to lift fragment 2 from Genscript plasmid, use |
| NO: 50 | | | with OSM362 |
| SEQ ID | OSM362 | TTTTTGACGACCGTCTGAA | |
| NO: 51 | | | |
| SEQ ID | OSM363 | AGTTTTGCTATTTTCAGAC | C.N. 57 to lift fragment 3 from Genscript plasmid, use |
| NO: 52 | | | with OSM364 |
| SEQ ID | OSM364 | gttgccgccgggcgttttttatggcgcgccTGGTTATGG | |
| NO: 53 | | TTAGTATTATCTAAATT | |
| SEQ ID | OSM402 | TAGAGCAAATACCTGATCTGG | C.N. 55 to lift fragment 1 from strain, use with OSM371 |
| NO: 54 | | | |
| SEQ ID | OSM371 | GTTGCCGCCGGGCGTTTTTTATGGCGCG | |
| NO: 55 | | CCAGCGGTAAGTTTTATAGAAAAT | |
| SEQ ID | OSM403 | TCTCTTAGATCCAGATCAGG | C.N. 55 to lift fragment 2 from strain, use with OSM370 |
| NO: 56 | | | |
| SEQ ID | OSM370 | ATCCTAGAAGCTTATCGAATTCGCGGCC | |
| NO: 57 | | GCAGGGGGGATATAGATTTATATATAA | |
| | | T | |
| SEQ ID | OSM335 | ATCCTAGAAGCTTATCGAATTCGCGGCC | C.N. 25 to lift from strain, use with OSM336 |
| NO: 58 | | GCACAAGATACATCACATTGAAGG | |
| SEQ ID | OSM336 | GTTGCCGCCGGGCGTTTTTTATGGCGCG | |
| NO: 59 | | CCCAATATATGCGATATACGCAAAAAC | |
| SEQ ID | OSM329 | ATCCTAGAAGCTTATCGAATTCGCGGCC | C.N. 64 to lift from strain, use with OSM330 |
| NO: 60 | | GCACAAATGGGCAGAGAGAG | |
| SEQ ID | OSM330 | GTTGCCGCCGGGCGTTTTTTATGGCGCG | |
| NO: 61 | | CCGATATTTCATTTTCTTCTTCCTCTCAC | |
| SEQ ID | OSM420 | ATCCTAGAAGCTTATCGAATTCGCGGCC | C.N. 66 to lift from strain, use with OSM421 |
| NO: 62 | | GCGGTGAATGTAAAAATATTGATACAG | |
| SEQ ID | OSM421 | GTTGCCGCCGGGCGTTTTTTATGGCGCG | |
| NO: 63 | | CCAATTTAGTCCTCCTGGGG | |
| SEQ ID | OSM31 | tgagttgattgcagtccagttac | C.N. 59 to lift fragment 1 from Gen9 construct, use with |
| NO: 64 | | | OSM32 |
| SEQ ID | OSM32 | GTTACCACCTACTATCTCTTCAGC | |
| NO: 65 | | | |
| SEQ ID | OSM33 | TCTTTAGCTGAAGAGATAGTAGGTG | C.N. 59 to lift fragment 2 from Gen9 construct, use with |
| NO: 66 | | | OSM34 |
| SEQ ID | OSM34 | tatggaggtcaggtatgatttaaatg | |
| NO: 67 | | | |
| SEQ ID | 0G0242 | catcactgaccatttaaatcatacctgacctccataCGAATG | p15-pDR110 backbone for C.N. 59, use with OSM28 |
| NO: 68 | | GCGATTTTCGTTCGTG | |
| SEQ ID | OSM28 | gcttgactccagcgtaactggactgcaatcaactcaCGATC | |
| NO: 69 | | AGACCAGTTTTTAAT | |
| SEQ ID | OSM386 | ATCCTAGAAGCTTATCGAATTC | C.N. 56 to lift fragment 1 from Genscript plasmid, use |
| NO: 70 | | | with OSM387 |
| SEQ ID | OSM387 | ATGTCATTCATAGCCAGAGC | |
| NO: 71 | | | |
| SEQ ID | OSM388 | AGATTTTAATGCTCTGGCTA | C.N. 56 to lift fragment 2 from Genscript plasmid, use |
| NO: 72 | | | with OSM389 |
| SEQ ID | OSM389 | CTCATCTTCTGATAATGGTCC | |
| NO: 73 | | | |
| SEQ ID | OSM390 | CAGCCGAACGGACCATT | C.N. 56 to lift fragment 3 from Genscript plasmid, use |
| NO: 74 | | | with OSM391 |
| SEQ ID | OSM391 | GTTGCCGCCGGGCGT | |
| NO: 75 | | | |
| SEQ ID | OLA489 | ATCCTAGAAGCTTATCGAATTCGCGGCC | C.N. 51 to lift from strain, use with OLA490 |
| NO: 76 | | GCACCTGCCTTCCTTTGATACAATTC | |
| SEQ ID | OLA490 | GTTGCCGCCGGGCGTTTTTTATGGCGCG | |
| NO: 77 | | CCTCAATTTATTGCTTTATTCATTGTGAC | |
| | | TG | |

For long systems (>10000 bases) when the donor strain was not available, the system was commercially synthesized in overlapping fragments (Table 4, “Cloning method” column). Systems amplified from genomic DNA or ordered as overlapping fragments were cloned into pSG1-rfp between the AscI and NotI sites using NEBuilder HiFI DNA Assembly cloning kit (NEB E5520S). The full list of sources used for cloning the systems into our model organisms is found in Table 4, including accessions of all strains ordered.

Transformation to B. subtilis was performed using MC medium as previously described (Wilson G A, Bott K F. (1968) Nutritional factors influencing the development of competence in the Bacillus subtilis transformation system. J Bacteriol. 95: 1439-49). MC medium was composed of 80 mM K₂HPO₄, 30 mM KH₂PO₄, 2% Glucose, 30 mM Trisodium citrate, 22 μg/ml Ferric ammonium citrate, 0.1% Casein Hydrolysate (CAA), 0.2% potassium glutamate. From an overnight starter of bacteria, 10 μl were diluted in 1 ml of MC medium supplemented with 10 μl 1M MgSO₄. After 3 hours of incubation (37° C., 200 rpm), 300 μl was transferred to a new 15 ml tube and ˜200 ng of plasmid DNA was added. The tube was incubated for another 3 hours (37° C., 200 rpm), and the entire reaction was plated on LB agar plates supplemented with 100 μg/ml spectinomycin and incubated overnight at 30° C.

For systems to be tested on E. coli, the cloned vector was transformed into E. coli MG1655 cells (ATCC 47076), and the resulting transformants were verified by PCR. For systems to be tested in B. subtilis, the cloned vector was transformed into B. subtilis BEST7003 cells. The system was integrated into the amyE locus, and resulting transformants were screened on starch plates for amylase-deficient phenotype. Whole-genome sequencing was then applied to all transformed B. subtilis and E. coli clones as described in (Ofir G, Melamed S, Sberro H, Silverman S, Doron S, Yaakov G, Mukamel Z, Sorek R. (2017) DISARM, a novel bacterial defense system with broad anti-phage activities. ibid) to verify system's integrity and lack of mutations.

As a negative control for transformation into B. subtilis, a transformant with an empty plasmid, containing only the spectinomycin resistance gene in the amyE locus, was used. As a negative control for transformation into E. coli, the wild-type E. coli MG1655 carrying an empty plasmid was used.

For strains with gene deletions and point mutations, plasmids containing systems with these deletions/mutations were commercially synthesized by Genscript. The mutated systems were transformed into B. subtilis and E. coli as described above, and clones used were fully sequenced to verify proper integration and sequence of the mutated systems.

Phage Strains, Cultivation, and Plaque Assay

The following B. subtilis phages were obtained from the Bacillus Genetic Stock Center (BGSC): SPO1 (BGSCID 1P4), phi3T (BGSCID 1L1), SPβ (BGSCID 1L5), SPR (BGSCID 1L56), phi105 (BGSCID 1L11), rho14 (BGSCID 1L15), and SPP1 (BGSCID 1P7). Phage phi29 was obtained from the DSMZ (DSM 5546). Phages SBSphiJ and SBSphiC were isolated by us from mixed soil and leaves samples on B. subtilis BEST7003. For this, soil and leaves samples were added to a log phase B. subtilis BEST7003 culture and incubated overnight to enrich for B. subtilis phages. The enriched sample was centrifuged and filtered through 0.2 μm filters, and the filtered supernatant was used to perform double layer plaque assays as described in Kropinski et al. (Kropinski A M, Mazzocco A, Waddell T E, Lingohr E, Johnson R P. Enumeration of Bacteriophages by Double Agar Overlay Plaque Assay. In: Clokie M R J, Kropinski A M, editors. Bacteriophages: Methods and protocols. NY: Humana Press; 2009. pp. 69-76). Single plaques that appeared after overnight incubation were picked, re-isolated 3 times, and amplified as described below.

E. coli phages (T4, T7, lambda-vir) were kindly provided by U. Qimron. Phages SECphi17, SECphi18 and SECphi27 were isolated as described in Wommack et al. (Wommack K E, Williamson K E, Helton R R, Bench S R, Winget D M. Methods for the Isolation of Viruses from Environmental Samples. In: Clokie M R J, Kropinski A M, editors. Bacteriophages: Methods and protocols. NY: Humana Press; 2009. pp. 3-14) from sewage samples on E. coli MG1655. 0.2 μm filtered concentrated sewage samples were used to perform double layer plaque assays, individual plaques were picked, re-isolated 3 times, and amplified as described below.

All phages isolated by us were Illumina sequenced following a library prep using the Nextera protocol (Baym M, Kryazhimskiy S, Lieberman T D, Chung H, Desai M M, Kishony R. (2015) Inexpensive multiplexed library preparation for megabase-sized genomes. PLoS One. 10: e0128036) and assembled using SPAdes v. 3.10.1 using the -careful and -cov-cutoff auto modifiers (Bankevich et al. (2012) A New Genome Assembly Algorithm and Its Applications to Single-Cell Sequencing. J Comput Biol.; 19: 455-477. doi:10.1089/cmb.2012.0021). Assembled genomes and raw reads were deposited in the European Nucleotide Archive (ENA) under study accession PRJEB23070. Phage classification was done according to sequence homology to the closest known similar phage. Phage SECphi17 (ENA ERS1981053) has a 5,538 bp genome and its closest relative is Coliphage WA3 (GenBank DQ079897.1, 66% coverage, 81% identity), indicating it is an ssDNA phage of the Microviridae family. Phage SECphi18 (ENA ERS1981054) has a 44,798 bp genome and its closest relative is Escherichia phage Gluttony (GenBank KX534336.1, 92% coverage, 93% identity), indicating it is a member of the Siphoviridae family. Phage SECphi27 (ENA ERS1981055) has a 51,811 bp genome, and its closest relative is Escherichia phage vB_Eco_swan01 (GenBank LT841304.1, 91% coverage, 98% identity), indicating it is a member of the Siphoviridae family. Phage SBSphiJ (ENA ERS1981056) has a 156,875 bp genome, and its closest relative is Bacillus phage Grass (GenBank KF669652.1, 91% coverage, 95% identity), indicating it is a member of the family Myoviridae. Phage SBSphiC (ENA ERS1981057) has a 144,651 bp genome, and its closest relative is Bacillus phage SP10 (GenBank AB605730.1, 94% coverage, 90% identity), indicating it is a member of the Myoviridae family. Siphoviridae and Myoviridae phage morphologies were verified by electron microscopy (EM). For the EM experiments, phage lysates were blotted onto copper grids, stained using uranyl acetate 2%, and visualized in FEI Tecnai T12 transmitting electron microscope.

Phages were propagated on either E. coli MG1655 or B. subtilis BEST7003 using the plate lysate method as previously described (Fortier L-C, Moineau S. (2009) Phage production and maintenance of stocks, including expected stock lifetimes. Methods Mol Biol. 501: 203-19). Lysate titer was determined using the small drop plaque assay method as previously described (Mazzocco A, Waddell T E, Lingohr E, Johnson R P. (2009) Enumeration of bacteriophages using the small drop plaque assay system. [Internet]. Methods in molecular biology (Clifton, N.J.). pp. 81-5). Bacteria were mixed with MMB agar (LB+0.1 mM MnCl₂+5 mM MgCl₂+5 mM CaCl₂+0.5% agar), and serial dilutions of phage lysate in MMB were dropped on top of them. After the drops dried up, plates were incubated at room temperature overnight. Efficiency of plating (EOP) was measured by performing small drop plaque assay with the same phage lysate on control bacteria and bacteria containing the candidate defense system, and comparing the ratio of plaque formation.

To determine the number of infective centers during infection with T7 phage of control bacteria and bacteria containing type I or type II Zorya (Defense Systems Ia and Ib, respectively), a modified version of the technique described in (Sing et al., (19909) J. Gen. Microbiol. 136:807-1815) as used. Zorya-lacking E. coli MG1655 or Zorya-containing cells were infected with T7 phage at MOI 0.05 and incubated for 10 min at 37° C. to allow adsorption. Cells with adsorbed phages were then centrifuged (1 min, 14,000 rpm) at 4° C., washed once with ice-cold MMB medium, and resuspended in 200 mlice-cold MMB medium. Then, 100 ml aliquots of 10-fold dilutions of resuspended phage-infected cells were mixed with 100 ml of a Zorya-lacking E. coli MG1655 culture grown to O.D. 0.3. The mixture was plated using the double agar over-lay method and infection centers (plaques) were counted after overnight incubation in room temperature.

For the liquid culture infection with T7 phage, overnight cultures of Zorya-lacking E. coli MG1655 or Zorya-containing cells were diluted 1:100 in MMB medium. 180 ml volumes of the diluted culture were dispersed into wells in a 96-well plate and grown at 37° C. with vigorous shaking until early log phase (O.D.600 0.3). 20 mlofT7 phage lysate were added at multiplicities of infection 0.05, 0.5 and 5 in three replicates. Optical density measurements at a wavelength of 600 nm were taken every 15 min using a TECAN Infinite 200 plate reader in a 96-well plate.

Transformation Efficiency Assay

Transformation was performed using the MC medium as described above. To test plasmid transformation efficiency, the episomal Bacillus plasmid pHCMC05 was used (Titok M., Chapuis J, Selezneva Y., Lagodich A., Prokulevich V., Ehrlich S., Jannière L. (2003) Bacillus subtilis soil isolates: plasmid replicon analysis and construction of a new theta-replicating vector. Plasmid. 49: 53-62). Transformation efficiency was calculated by dividing the number of transformants that grew on LB plates containing 5 μg/ml chloramphenicol by the live count on LB plates.

DNA-Seq and RNA-Seq

DNA was extracted from bacteria using Qiagen DNeasy blood and tissue kit (Qiagen 69504). DNA libraries were constructed using the Nextera library preparation protocol as previously published (Baym M, Kryazhimskiy S, Lieberman T D, Chung H, Desai M M, Kishony R. (2015) Inexpensive multiplexed library preparation for megabase-sized genomes. PLoS One. 10: e0128036). RNA-seq was performed with the NEBNext Ultra Directional RNA Library Prep Kit (NEB, E7420) according to the manufacturer's instructions with modifications as previously described (Dar D, Shamir M, Mellin J R, Koutero M, Stern-Ginossar N, Cossart P, Sorek R. (2016) Term-seq reveals abundant ribo-regulation of antibiotics resistance in bacteria. Science. 352: aad9822-aad9822). Prior to library preparation, equal amounts of extracted RNA from 3-7 strain samples were pooled together and processed as a single library. All libraries were sequenced using the Illumina NextSeq500. The sequencing reads were aligned to the reference genomes of B. subtilis BEST7003 (Genbank: AP012496) and E. coli MG1655 (Genbank: NC_000913), and to the plasmid sequence of each system, using Novoalign 3.02.02 (Novocraft Technologies Sdn Bhd, http://www.novocraft.com) with the default parameters and [-r Random]. The coverage along the reference genomes was calculated, to check if each system exists in the genome (DNA-seq) or expressed (RNA-seq). The pooled RNA library was sequenced to a depth of 5 million reads per sample and later aligned to the reference genomes as described.

### Example 1

### Identification of New Bacterial Defense Systems

Objective:

To comprehensively identify new defense systems enriched within defense islands, in an attempt to systematically map the arsenal of defense systems that are at the disposal of bacteria and archaea in their fight against phages. Anti-phage defense systems were found to be frequently physically clustered in bacterial and archaeal genomes such that, for example, genes comprising restriction enzymes commonly reside in the vicinity of genes comprising infection systems and other phage resistance systems. The observation that defense systems are clustered in genomic “defense islands” has led to the suggestion that genes of unknown function residing within such defense islands may also participate in anti-phage defense.

Methods:

The first step in this discovery effort was to identify gene families that are enriched near known defense systems in the microbial pan-genome (FIG. 1A). For this, 14,083 protein families (pfams) were analyzed in >45,000 available bacterial and archaeal genomes (overall comprising >120 million genes). Each pfam represents a set of genes sharing a common protein domain. For each pfam, the prevalence of its member genes to reside in the vicinity (10 genes from each side) of one or more known defense genes was calculated (FIG. 1B). Pfams were selected that had at least 65% of their member genes found next to defense genes, and that their member genes appeared in diverse defense contexts in different genomes (at least 10% variability; FIG. 1B). These thresholds were selected as they capture the majority of pfams that comprise known defense systems (FIGS. 1A-1C; Table 1). The resulting set of 277 candidate pfams was supplemented by 58 additional gene families that were previously predicted to be associated with known defense systems (Makarova K S, Wolf Y I, Snir S, Koonin E V. (2011) Defense islands in bacterial and archaeal genomes and prediction of novel defense systems. J Bacteriol. 193: 6039-56), altogether yielding a list of 335 candidate gene families (Table 2).

To check whether the defense-associated pfams belong to multi-gene systems, each such pfam was used as an anchor around which commonly associated genes were searched for (FIG. 1A). For this, all the neighboring genes (10 genes from each side) from all the genomes in which members of the anchor pfam occurred were collected, and these genes were clustered based on sequence homology. Cassettes of gene clusters were then searched for that showed conserved order across multiple different genomes, marking such cassettes as candidate multi-gene systems (FIG. 1A).

Results:

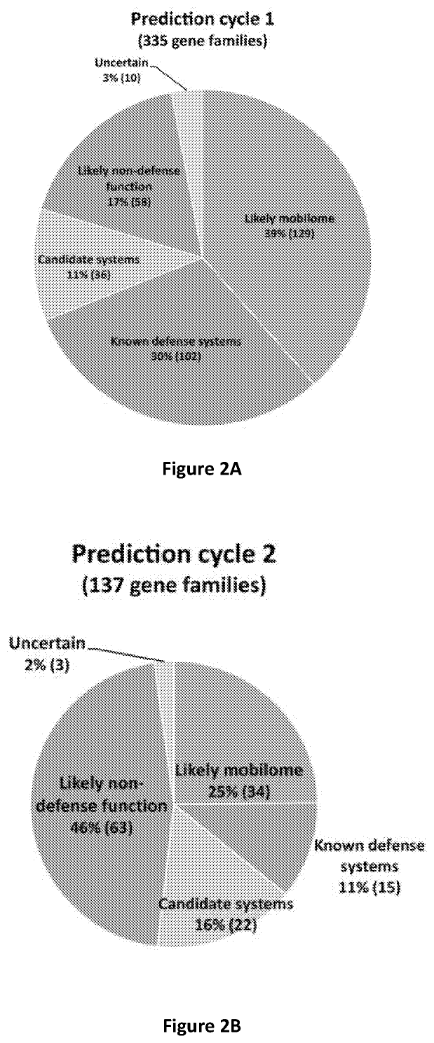

The gene annotations in the resulting candidate systems were manually inspected in order to filter out likely false predictions. It was found that 39% of the cases represented non-defense, mobile genetic elements that are known to co-localize to defense islands (Table 2). Additionally, 30% of the resulting candidate defense systems found represented known defense systems whose pfams were not included in the original set of known defense pfams, and 17% belonged to operons probably performing metabolic functions or other functions not associated with defense (FIGS. 2A and 2B).

The remaining systems possibly represent putative new defense systems. To expand the predictions with new pfams that may be specifically enriched next to the putative new defense systems, a second prediction cycle was performed, this time adding the members of the predicted new systems to the positive defense pfam set (FIG. 1A).

Altogether, 41 candidate single-gene or multi-gene systems were retrieved from this analysis (Table 3). This set of systems was further filtered to remove systems that were largely confined to a specific clade (e.g., systems appearing only in cyanobacteria or only in actinobacteria), resulting in a set of 28 candidate systems that showed broad phylogenetic distribution.

### Example 2

### Verification of New Bacterial Defense Systems

Objective:

To experimentally verify the candidate defense systems identified in Example 1, as having defense system activity.

Methods:

Two bacteria strains were selected for verification of candidate defense systems, Escherichia coli str. MG1655 and Bacillus subtilis str. BEST7003. These bacteria were utilized as models to experimentally examine whether the predicted systems confer defense against phage (FIG. 3A). None of the candidate new systems are naturally present in the genomes of these two bacteria. For each candidate system, source organisms were selected from which the system was taken and heterologously cloned into one of the model organisms. To increase the probability that the cloned system would be compatible and functionally expressed within the receiving bacterium, systems were selected from mesophilic organisms as close phylogenetically as possible to E. coli or to B. subtilis, and included the upstream and downstream intergenic regions so that promoters, terminators or other regulatory sequences would be preserved. Where possible at least two instances of each system were used (from two different source genomes), to account for the possibility that some systems may not be active in their source organism. The DNA of each system, spanning the predicted genes and the intergenic spaces, was synthesized or amplified from the source genome (donor species) and cloned either to E. coli (on a plasmid) or to B. subtilis (genomically integrated). As a control, the procedure was repeated with 5 known defense systems (instances of types I, II and III R-M systems, type III toxin/antitoxin system and an infection gene of the AbiH family) for which source organisms were similarly selected and cloning was performed into B. subtilis, as well a 6^{th} control comprised of the recently discovered DISARM defense system.

Results:

Altogether, heterologously cloning of 61 representative instances of the 28 candidate new systems was attempted, and successful cloning was verified by whole genome sequencing (Table 4). For 27 of these 28 systems there was at least one candidate locus for which cloning was successful, and RNA-seq of the transformants showed that for 26 of the systems, at least one of the candidates was expressed in the receiving E. coli or B. subtilis strain.

The engineered bacteria were then challenged by an array of phages consisting of 10 B. subtilis and 6 E. coli phages, spanning the three major families of tailed dsDNA phages (myo-, sipho- and podo-phages), as well as one ssDNA phage infecting E. coli (FIGS. 3B, 3C, and 3D). Measuring phage efficiency of plating (EOP) on system-containing bacteria vs. control cells, it was found that 9 of the 26 tested systems (35%) showed protection from infection by at least one phage (FIGS. 3B, 3C, 3D, 3E, and 3F). In comparison, three of the six positive control systems showed defense, with the remaining 3 showing no protection against the 10 B. subtilis phages tested.

The 9 verified new systems were named after protective deities from various world mythologies. These defense systems are comprised of between 1 and 5 genes and span between 2 and 12 kb of genomic DNA (Table 6; Table 7).

TABLE 6
Composition of Defense Systems Reported

| | | | | | No. (%) | |
|---|---|---|---|---|---|---|
| | | | | # of | of | |
| | | | | instances | genomes | |
| | | | | detected | in which | |
| | | | | within | system | |
| System | Operon | Associated domains | Domain annotations | microbes | is found | comments |
| Thoeris | ThsAB | pfam13289, pfam14519, | SIR2, Macro domain, | 2,099 | 2070 | Membrane |
| | | pfam08937, pfam13676 | TIR domain | | (4.0%) | associated |
| | | | | | | (sometime) |
| Hachiman | HamAB | pfam08878, COG1204, | Helicase | 1,781 | 1742 | |
| | | pfam00270, pfam0271 | | | (3.4%) | |
| Shedu | SduA | pfam14082 | Nuclease | 1,246 | 1191 | |
| | | | | | (2.3%) | |
| Gabija | GajAB | pfam13175, COG3593, | ATPase, helicase, | 4,598 | 4360 | |
| | | pfam13361, pfam00580, | nuclease | | (8.5)% | |
| | | COG0210, pfam13245 | | | | |
| Septu | PtuAB | pfam13304, COG3950, | ATPase, HNH nuclease | 2,506 | 2117 | |
| | | pfam13395, pfam01844 | | | (4.1%) | |
| Lamassu | LmuAB | pfam14130, pfam02463 | SMC ATPase N-terminal | 697 | 682 | |
| | | | domain | | (1.3%) | |
| Zorya | ZorABCD | pfam01618, pfam13677, | MotA/ExbB, MotB, | 1,173 | 1055 | Membrane |
| (type I) | | pfam00691, COG1360, | helicase, Mrr-like | | (2.1%) | associated |
| | | pfam15611, pfam00176, | nuclease | | | |
| | | pfam00271, COG0553, | | | | |
| | | pfam04471 | | | | |
| Zorya | ZorABE | pfam01618, pfam13677, | MotA/ExbB, MotB, | 656 | 655 | Membrane |
| (type II) | | pfam00691, COG1360, | HNH nuclease | | (1.3%) | associated |
| | | COG3183, pfam01844 | | | | |
| Kiwa | KwaAB | pfam16162 | No annotated domain | 934 | 924 | Membrane |
| | | | | | (1.8%) | associated |
| Druantia | DruABC | pfam14236, pfam00270, | Helicase, methylase | 1,342 | 1321 | |
| | DE (type | pfam00271, pfam09369, | | | (2.6%) | |
| | I) | pfam00145, COG1205, | | | | |
| | DruMFG | COG0270 | | | | |
| | E (type II) | | | | | |
| | DruHE | | | | | |
| | (III) | | | | | |
| Wadjet | JetABCD | pfam11855, pfam09660, | MukBEF condensin, | 3,173 | 2880 | |
| | | pfam13835, pfam09661, | topoisomerase VI | | (5.6%) | |
| | | pfam13555, pfam13558, | | | | |
| | | COG4913, COG1196, | | | | |
| | | pfam11795, pfam09983, | | | | |
| | | pfam11796, pfam09664, | | | | |
| | | COG4924 | | | | |

TABLE 7
Verified Defense Systems

| | | | | Median | |
|---|---|---|---|---|---|
| | | Associated | | gene size | |
| System | Genes | domains | Domain annotation | (aa) | Comment |
| Thoeris | ThsA | pfam13289, | Sir2/Macro domain | 477 | In some cases N- |
| | | pfam14519 | (NAD binding) | | terminal |
| | | | | | transmembrane |
| | | | | | helices |
| | ThsB | pfam08937 | TIR domain | 195 | Often multiple |
| | | | | | ThsB genes in the |
| | | | | | locus |
| Hachiman | HamA | pfam08878 | Unknown function | 269 | |
| | | (DUF1837) | | | |
| | HamB | COG1204, | Helicase | 867 | |
| | | pfam00270, | | | |
| | | pfam00271 | | | |
| Shedu | SduA | pfam14082 | Endonuclease NucS | 367 | |
| | | (DUF4263), | | | |
| | | pfam01939 | | | |
| Gabija | GajA | pfam13175, | ATPase domain, | 670 | |
| | | COG3593 | Predicted ATP- | | |
| | | | dependent | | |
| | | | endonuclease | | |
| | GajB | pfam04257, | UvrD helicase | 598 | |
| | | pfam00580- | | | |
| | | pfam13361 | | | |
| Septu | PtuA | pfam13304, | AAA domain, | 452 | In some cases has |
| | | pfam02463 | RecF/RecN/SMC N | | N-terminal |
| | | | terminal domain | | predicted HNH |
| | | | | | nuclease domain |
| | PtuB | pfam13395, | HNH endonuclease | 219 | |
| | | pfam01844 | | | |
| Lamassu | LmuA | pfam14130 | Unknown function | 361 | |
| | | (DUF4297) | | | |
| | LmuB | pfam02463 | RecF/RecN/SMC N | 559 | |
| | | | terminal domain | | |
| Zorya | ZorA | pfam01618 | MotA/ExbB/TolQ | 550 | Predicted |
| type I | | | | | transmembrane |
| | | | | | helices |
| | ZorB | pfam13677- | MotB | 217 | Predicted |
| | | pfam00691 | | | transmembrane |
| | | | | | helix |
| | ZorC | pfam15611 | EH signature | 470 | |
| | ZorD | pfam00176- | SWI2/SNF2 helicase | 1080 | |
| | | pfam00271 | | | |
| Zorya | ZorE | COG3183, | HNH endonuclease | 367 | |
| type II | | pfam01844 | | | |
| Kiwa | KwaA | No domain | | 194 | Predicted |
| | | annotation | | | transmembrane |
| | | | | | helices |
| | KwaB | pfam16162 | Unknown function | 315 | |
| | | (DUF4868) | | | |
| Druantia | DruA | pfam14236 | Unknown function | 404 | |
| type I | | (DUF4338) | | | |
| | DruB | No domain | | 531 | |
| | | annotation | | | |
| | DruC | No domain | | 627 | |
| | | annotation | | | |
| | DruD | No domain | | 347 | |
| | | annotation | | | |
| | DruE | pfam00270, | Helicase | 2104 | |
| | | pfam00271, | | | |
| | | pfam09369 | | | |
| | | (DUF1998) | | | |
| Druantia | DruM | pfam00145, | Cytosine methylase | 386 | |
| type II | | COG0270 | | | |
| | DruF | No domain | | 852 | |
| | | annotation | | | |
| | DruG | No domain | | 711 | |
| | | annotation | | | |
| Druantia | DruH | No domain | | 1079 | |
| type III | | annotation | | | |
| Wadjet | JetA | pfam11855 | Unknown functions | 488 | MukF-like |
| | | (DUF3375)/ | | | |
| | | pfam09660 | | | |
| | | (DUF2397) | | | |
| | JetB | pfam13835 | Unknown functions | 247 | MukE-like |
| | | (DUF4194)/ | | | |
| | | pfam09661 | | | |
| | | (DUF2398) | | | |
| | JetC | pfam13555- | ATP binding domain, | 1476 | MukB-like |
| | | pfam13558 | Putative exonuclease | | |
| | | | SbcC | | |
| | JetD | pfam11795 | Unknown functions | 347 | Topoisomerase VI |
| | | (DUF3322)- | | | predicted by |
| | | pfam09983 | | | structural |
| | | (DUF2220)/ | | | similarities |
| | | pfam11796 | | | |
| | | (DUF3323)- | | | |
| | | pfam09664 | | | |
| | | (DUF2399) | | | |

Where possible, system consistency was verified by testing for phage resistance in systems where individual genes were deleted (FIG. 3G). We found between several hundreds and several thousand representations of each of the defense systems in sequenced microbial genomes, usually with broad phylogenetic distribution (FIG. 3H; Tables 8-17). Most systems were detected in >10 taxonomic phyla, and 7 of them appear in archaea (FIG. 3H).

Some of the systems seem to target a specific class of phages (e.g., the Thoeris system appears to specifically protect from myophages), while others, such as the Hachiman system, provide broader defense (FIG. 3B). The genes comprising the new systems encode many protein domains that are commonly present in antiviral systems such as CRISPR-Cas and RNAi, including helicases, nucleases, and nucleic acid binding domains, in addition to many domains of unknown function and also atypical domains as described herein. Three of the systems contain membrane-associated proteins as predicted by the presence of multiple transmembrane helices. Further functional analyses was performed for a selected set of systems.

### Example 3

### Genomic Identification and Analysis of the ZORYA System

Objective:

Identify the candidate defense system based on the enrichment of the anchor pfam15611.

Method:

ZORYA was one of the systems identified in the first round of prediction. The anchor gene family for ZORYA was pfam15611. This pfam had a defense score of 0.98 and a diversity score of 0.55.

Cloning of ZORYA into E. coli MG1655—A cloning vector for large fragments was constructed by assembling the p15a origin of replication (ori) from pACYCDuet-1 and the amyE integration cassette from plasmid pDR110 (see https://www(dot)ncbi(dot)nlm(dot)nih(dot)gov/pmc/articles/PMC3814332/#pgen.1003892.s005). The p15a ori was amplified using primers OGO174+OGO175 (a list of all primers is provided in Table 5). The amyE integration cassette was amplified using primers OGO176+OGO185. The two fragments were assembled and transformed into E. coli cells using Gibson assembly cloning kit (NEB E5510S), and assembled plasmids were verified by restriction pattern and full sequencing.

The backbone of the above described vector was amplified using primers OGO309+OGO310, adding to it a BamHI restriction site and a terminator site. The multiple cloning site of plasmid pBS1C (received from the BGSC) was amplified using primers OGO311+OGO312. Both fragments were digested using AscI and BamHI, ligated using T4 ligase and transformed into E. coli, resulting in plasmid pSG1-rfp. The plasmid was digestion verified and fully sequenced.

A representative ZORYA operon (locus) from E. coli E24377A (RefSeq accession NC_009801, coordinates 298890-307639 of the reverse strand, SEQ ID NO: XXXX) was commercially synthesized (GenScript) directly into pSG1-rfp between the AscI and NotI sites of the multiple cloning site. The cloned vector was transformed into E. coli MG1655 cells, and the resulting transformants were verified by PCR and whole genome sequencing.

Phage cultivation and plaque assays have been described above.

For analysis in solid cultures, Zorya-containing cells (Defense System Ia or Defense System Ib) were infected with T7 phage at MOI 0.05 and were incubated for 10 min at 37° C. to allow phage adsorption (FIG. 4G). Cells with adsorbed phages were then centrifuged and washed to eliminate unabsorbed phages. These T7-infected cells were mixed together with E. coli MG1655 cells that lacked a Zorya defense system, and the mixture was plated using an agar overlay method. Phage bursts from successful infections were visualized as a single infection center on a lawn from the T7-sensitive E. coli strain, enabling an evaluation of the number of phages that have adsorbed and completed a successful infection cycle. For analysis in liquid cultures, T7 infection in liquid cultures of E. coli cells containing type I (FIG. 4H) and type II (FIG. 4I) Zorya. Bacteria were infected at t=0 at multiplicities of infection (MOI) of 0.05, 0.5 and 5.

Results:

The Zorya system (named after a deity from the Slavic mythology) was identified based on the enrichment of the anchor pfam15611, representing a domain of unknown function, within defense islands. Pfam15611-containing gene clusters were previously reported as genomically associated with tellurium- and stress-resistance genes.

The computational analysis found this pfam to be part of a 4-gene system, zorABCD comprising ZorA, ZorB, ZorC, and ZorD polypeptides. Overall the ZORYA gene system encompasses ˜9 kb of DNA, with pfam15611 being the third gene in the system (zorC; FIG. 3C, and FIGS. 4A and 4B; Table 6). Initially 262 appearances of the ZORYA system were found in the dataset that was used in the automatic prediction.

A representative ZORYA operon from E. coli E24377A was cloned into E. coli MG1655 (FIG. 4C), which does not contain an endogenous ZORYA system. The correct insertion of the system into the E. coli MG1655 genome was verified by whole genome sequencing.

In the next step, the E. coli MG1655 containing the ZORYA system (type I) was challenged with phage from three families of the dsDNA tail phages Caudavirales: T4 of Myoviridae, T7 of the Podoviridae, and Lambda of the Siphoviridae. T4 and T7 are obligatory lytic phages, while Lambda is a temperate phage. In addition to the wild-type Lambda phage (lambda-wt), a mutant that is an obligatory lytic phage was also used (lambda-vir). The results showed that the type I ZORYA system provided protection against T7 phage (2-3 orders of magnitude), and lambda-vir phage (at least 2 orders of magnitude), manifested by reduction in plaque formation on the ZORYA-containing strains (FIG. 3E). Further analysis showed that the type I ZORYA system provided 10-10000-fold protection against infection by T7, SECphi27, and lambda-vir phages (FIGS. 3C and 3F). Further searches based on homologies to the first two genes in the system, zorA and zorB, revealed a second type of ZORYA, comprised of the 3 genes zorABE. (FIGS. 4D and 4E). A Type II Zorya system was cloned from E. coli ATCC8739 into E. coli MG1655 and yielded 10,000-fold defense against T7 and 10-fold against the ssDNA phage SECphi17 (FIG. 3C and FIGS. 4B and 4C).

The first two genes of the Zorya system, zorA and zorB, contain protein domains sharing sequence homology with motA and motB, respectively (FIG. 4B). MotA and MotB are inner membrane proteins that are part of the flagellar motor of bacteria. They assemble into a MotAB complex, which forms the stator of the flagellar motor (the static part within which the flagellar rotor swivels). The MotAB complex also forms the proton channel that provides the energy for flagellar rotation, coupling transport of protons into the cell with the rotation (FIG. 4F). While zorB shares the same size and domain organization with motB (including the pfam13677 and pfam00691 domains), zorA contains, in addition to the MotA domain (pfam01618), also a long C-terminal helical domain that is sometime identified as a “methyl-accepting chemotaxis domain” (COG0840). In addition to these two genes, Type I Zorya contains zorC, a gene of unknown function, and zorD, which is a large gene (encoding a ˜1200aa polypeptide) comprising a helicase domain. Type II Zorya lacks zorC and zorD and instead contains zorE, a smaller gene comprising an HNH-endonuclease domain.

Analysis of the Zorya Defense Systems was performed by performing phage infection of Zorya Defense System-containing bacteria cells (Defense System Ia or Defense System Ib) in solid and liquid cultures. Three replicates of cells containing type I Zorya, type II Zorya or no Zorya defense system are shown in FIGS. 4G-4I; data represent, for each replicate, the number of derived infection centers as a fraction from the average number of infection centers derived from Zorya-lacking (“No Zorya”) cells (see Methods). The results show that in cells containing a type I Zorya defense system, only an average of 17% of infection events lead to successful phage progeny, while in cells containing a type II Zorya defense system, 6% of the infected cells yield successful phage bursts. (FIG. 4G) For liquid culture infection, shown are three replicates for each MOI, with each curve showing an individual replicate. The delayed collapse of Zorya-containing cultures suggests that most of the infected Zorya-containing cells undergo abortive infection, while a minority of cells produces viable phage progeny, which eventually accumulate to a high MOI leading to a culture collapse.

Conclusion:

The Zorya defense system was identified in 1829 instances within 1663 sequenced bacterial genomes belonging to 12 phyla, marking this system as prevalent in at least 3% of sequenced bacteria (Table 8).

TABLE 8
ZORYA Defense System and Components Thereof
This table is presented in landscape orientation. Due to the number of columns, the
table has been split into parts. Part A includes the first set of columns and therefore should be
place to the left of subsequent parts (Part B and where relevant Part C). To avoid any doubt, the
rows of the table in each part line up once the tables are placed together.
(Part A)

| | A | B | C | D | E | F | G |
|---|---|---|---|---|---|---|---|
| 1 | assembly_accession | taxid | species_taxid | organism_name | infraspecific_name | phylum | genomic_accession |
| 2 | GCA_000017645.1 | 78245 | 280 | Xanthobacter autotrophicus Py2 | Py2 | Proteobacteria | CP000781.1 |
| 3 | GCA_000761245.1 | 285 | 285 | Comamonas testosteroni | JL40 | Proteobacteria | AWOR01000012.1 |
| 4 | GCA_000017205.1 | 381754 | 287 | Pseudomonas aeruginosa PA7 | PA7 | Proteobacteria | CP000744.1 |
| 5 | GCA_000259025.1 | 1134459 | 287 | Pseudomonas aeruginosa PADK2_CF510 | PADK2_CF510 | Proteobacteria | AJHI01000009.1 |
| 6 | GCA_000297315.1 | 1163394 | 287 | Pseudomonas aeruginosa ATCC 700888 | ATCC 700888 | Proteobacteria | AKZF01000392.1 |
| 7 | GCA_000297355.1 | 1163395 | 287 | Pseudomonas aeruginosa E2 | E2 | Proteobacteria | AKZH01000152.1 |
| 8 | GCA_000399805.1 | 1315978 | 287 | Pseudomonas aeruginosa VRFPA02 | VRFPA02 | Proteobacteria | AQHM01000100.1 |
| 9 | GCA_000467675.1 | 1350465 | 287 | Pseudomonas aeruginosa VRFPA03 | VRFPA03 | Proteobacteria | ATNK01000038.1 |
| 10 | GCA_000480705.1 | 1402563 | 287 | Pseudomonas aeruginosa BL22 | BL22 | Proteobacteria | KI518786.1 |
| 11 | GCA_000480725.1 | 1402562 | 287 | Pseudomonas aeruginosa BL21 | BL21 | Proteobacteria | KI518793.1 |
| 12 | GCA_000480745.1 | 1402561 | 287 | Pseudomonas aeruginosa BL20 | BL20 | Proteobacteria | KI518807.1 |
| 13 | GCA_000480965.1 | 1402550 | 287 | Pseudomonas aeruginosa BL09 | BL09 | Proteobacteria | KI518889.1 |
| 14 | GCA_000481705.1 | 1402500 | 287 | Pseudomonas aeruginosa X13273 | X13273 | Proteobacteria | KI519199.1 |
| 15 | GCA_000481765.1 | 1402497 | 287 | Pseudomonas aeruginosa 19660 | 19660 | Proteobacteria | KI519236.1 |
| 16 | GCA_000481825.1 | 1402494 | 287 | Pseudomonas aeruginosa S54485 | S54485 | Proteobacteria | KI519256.1 |
| 17 | GCA_000482005.1 | 1163395 | 287 | Pseudomonas aeruginosa E2 | E2 | Proteobacteria | KI519307.1 |
| 18 | GCA_000496325.1 | 1408193 | 287 | Pseudomonas aeruginosa VRFPA05 | VRFPA05 | Proteobacteria | AXZJ01000095.1 |
| 19 | GCA_000520195.1 | 1402581 | 287 | Pseudomonas aeruginosa PS42 | PS42 | Proteobacteria | KI914449.1 |
| 20 | GCA_000531435.1 | 1407059 | 287 | Pseudomonas aeruginosa PA38182 | PA38182 | Proteobacteria | HG530068.1 |
| 21 | GCA_000531435.1 | 1407059 | 287 | Pseudomonas aeruginosa PA38182 | PA38182 | Proteobacteria | HG530068.1 |
| 22 | GCA_000558345.1 | 1441931 | 287 | Pseudomonas aeruginosa VRFPA09 | VRFPA09 | Proteobacteria | JAAO01000047.1 |
| 23 | GCA_000611995.2 | 1437873 | 287 | Pseudomonas aeruginosa PA99 | PA99 | Proteobacteria | KK111873.1 |
| 24 | GCA_000629065.1 | 1447541 | 287 | Pseudomonas aeruginosa BWH033 | BWH033 | Proteobacteria | KK212100.1 |
| 25 | GCA_000629285.1 | 1447530 | 287 | Pseudomonas aeruginosa 3575 | 3575 | Proteobacteria | KK212903.1 |
| 26 | GCA_000629505.1 | 1418240 | 287 | Pseudomonas aeruginosa BWH054 | BWH054 | Proteobacteria | KK213223.1 |
| 27 | GCA_000689435.1 | 1336203 | 287 | Pseudomonas aeruginosa MH38 | MH38 | Proteobacteria | CBTQ010000132.1 |
| 28 | GCA_000689435.1 | 1336203 | 287 | Pseudomonas aeruginosa MH38 | MH38 | Proteobacteria | CBTQ010000056.1 |
| 29 | GCA_000705175.1 | 1441459 | 287 | Pseudomonas aeruginosa C2773C | C2773C | Proteobacteria | JGZW01000198.1 |
| 30 | GCA_000829885.1 | 287 | 287 | Pseudomonas aeruginosa | FRD1 | Proteobacteria | CP010555.1 |
| 31 | GCA_000981825.1 | 287 | 287 | Pseudomonas aeruginosa | Carb01 63 | Proteobacteria | CP011317.1 |
| 32 | GCA_000981825.1 | 287 | 287 | Pseudomonas aeruginosa | Carb01 63 | Proteobacteria | CP011317.1 |
| 33 | GCA_001180345.1 | 287 | 287 | Pseudomonas aeruginosa | P1_London_28_IMP_1_04_05 | Proteobacteria | CVUY01000300.1 |
| 34 | GCA_001180345.1 | 287 | 287 | Pseudomonas aeruginosa | P1_London_28_IMP_1_04_05 | Proteobacteria | CVUY01000281.1 |
| 35 | GCA_001180365.1 | 287 | 287 | Pseudomonas aeruginosa | P2_London_28_IMP_1_06_05 | Proteobacteria | CVWC01000358.1 |
| 36 | GCA_001180365.1 | 287 | 287 | Pseudomonas aeruginosa | P2_London_28_IMP_1_06_05 | Proteobacteria | CVWC01000094.1 |
| 37 | GCA_001180385.1 | 287 | 287 | Pseudomonas aeruginosa | P3_North_West_16_VIM_2_07_06 | Proteobacteria | CVVV01000260.1 |
| 38 | GCA_001180385.1 | 287 | 287 | Pseudomonas aeruginosa | P3_North_West_16_VIM_2_07_06 | Proteobacteria | CVVV01000250.1 |
| 39 | GCA_001180405.1 | 287 | 287 | Pseudomonas aeruginosa | P4_London_1_VIM_2_10_07 | Proteobacteria | CVWA01000298.1 |
| 40 | GCA_001180405.1 | 287 | 287 | Pseudomonas aeruginosa | P4_London_1_VIM_2_10_07 | Proteobacteria | CVWA01000358.1 |
| 41 | GCA_001180425.1 | 287 | 287 | Pseudomonas aeruginosa | P5_London_26_VIM_2_01_09 | Proteobacteria | CVWS01000233.1 |
| 42 | GCA_001180445.1 | 287 | 287 | Pseudomonas aeruginosa | E1_London_17_VIM_2_02_09 | Proteobacteria | CWGH01000216.1 |
| 43 | GCA_001180445.1 | 287 | 287 | Pseudomonas aeruginosa | E1_London_17_VIM_2_02_09 | Proteobacteria | CWGH01000243.1 |
| 44 | GCA_001180465.1 | 287 | 287 | Pseudomonas aeruginosa | E2_London_17_VIM_2_02_09 | Proteobacteria | CVUZ01000030.1 |
| 45 | GCA_001180485.1 | 287 | 287 | Pseudomonas aeruginosa | E3_London_17_VIM_2_03_09 | Proteobacteria | CVVB01000221.1 |
| 46 | GCA_001180485.1 | 287 | 287 | Pseudomonas aeruginosa | E3_London_17_VIM_2_03_09 | Proteobacteria | CVVB01000215.1 |
| 47 | GCA_001180505.1 | 287 | 287 | Pseudomonas aeruginosa | E4_London_17_VIM_2_03_09 | Proteobacteria | CVUK01000139.1 |
| 48 | GCA_001180505.1 | 287 | 287 | Pseudomonas aeruginosa | E4_London_17_VIM_2_03_09 | Proteobacteria | CVUK01000152.1 |
| 49 | GCA_001180525.1 | 287 | 287 | Pseudomonas aeruginosa | P6_East_of_England_6_IMP_1_03_09 | Proteobacteria | CWFP01000032.1 |
| 50 | GCA_001180525.1 | 287 | 287 | Pseudomonas aeruginosa | P6_East_of_England_6_IMP_1_03_09 | Proteobacteria | CWFP01000037.1 |
| 51 | GCA_001180545.1 | 287 | 287 | Pseudomonas aeruginosa | P7_London_17_VIM_2_06_09 | Proteobacteria | CVXP01000253.1 |
| 52 | GCA_001180545.1 | 287 | 287 | Pseudomonas aeruginosa | P7_London_17_VIM_2_06_09 | Proteobacteria | CVXP01000219.1 |
| 53 | GCA_001180565.1 | 287 | 287 | Pseudomonas aeruginosa | P8_1_South_East_10_VIM_2_07_09 | Proteobacteria | CWET01000247.1 |
| 54 | GCA_001180565.1 | 287 | 287 | Pseudomonas aeruginosa | P8_1_South_East_10_VIM_2_07_09 | Proteobacteria | CWET01000222.1 |
| 55 | GCA_001180585.1 | 287 | 287 | Pseudomonas aeruginosa | P9_East_of_England_6_IMP_13_08_09 | Proteobacteria | CWEU01000015.1 |
| 56 | GCA_001180585.1 | 287 | 287 | Pseudomonas aeruginosa | P9_East_of_England_6_IMP_13_08_09 | Proteobacteria | CWEU01000241.1 |
| 57 | GCA_001180605.1 | 287 | 287 | Pseudomonas aeruginosa | P10_London_6_VIM_2_09_09 | Proteobacteria | CVVA01000108.1 |
| 58 | GCA_001180605.1 | 287 | 287 | Pseudomonas aeruginosa | P10_London_6_VIM_2_09_09 | Proteobacteria | CVVA01000120.1 |
| 59 | GCA_001180625.1 | 287 | 287 | Pseudomonas aeruginosa | P11_London_17_VIM_2_10_09 | Proteobacteria | CVVH01000136.1 |
| 60 | GCA_001180625.1 | 287 | 287 | Pseudomonas aeruginosa | P11_London_17_VIM_2_10_09 | Proteobacteria | CVVH01000150.1 |
| 61 | GCA_001180645.1 | 287 | 287 | Pseudomonas aeruginosa | P12_London_17_VIM_2_11_09 | Proteobacteria | CVUP01000157.1 |
| 62 | GCA_001180645.1 | 287 | 287 | Pseudomonas aeruginosa | P12_London_17_VIM_2_11_09 | Proteobacteria | CVUP01000141.1 |
| 63 | GCA_001180665.1 | 287 | 287 | Pseudomonas aeruginosa | P13_London_14_VIM_2_12_09 | Proteobacteria | CVUL01000136.1 |
| 64 | GCA_001180665.1 | 287 | 287 | Pseudomonas aeruginosa | P13_London_14_VIM_2_12_09 | Proteobacteria | CVUL01000126.1 |
| 65 | GCA_001180685.1 | 287 | 287 | Pseudomonas aeruginosa | P14_London_17_VIM_2_01_10 | Proteobacteria | CVVT01000217.1 |
| 66 | GCA_001180685.1 | 287 | 287 | Pseudomonas aeruginosa | P14_London_17_VIM_2_01_10 | Proteobacteria | CVVT01000236.1 |
| 67 | GCA_001180705.1 | 287 | 287 | Pseudomonas aeruginosa | P15_London_17_02_10 | Proteobacteria | CVVW01000231.1 |
| 68 | GCA_001180705.1 | 287 | 287 | Pseudomonas aeruginosa | P15_London_17_02_10 | Proteobacteria | CVVW01000262.1 |
| 69 | GCA_001180725.1 | 287 | 287 | Pseudomonas aeruginosa | P16_Lonon_17_VIM_2_02_10 | Proteobacteria | CVVD01000204.1 |
| 70 | GCA_001180725.1 | 287 | 287 | Pseudomonas aeruginosa | P16_Lonon_17_VIM_2_02_10 | Proteobacteria | CVVD01000193.1 |
| 71 | GCA_001180745.1 | 287 | 287 | Pseudomonas aeruginosa | P17_North_West_14_VIM_2_03_10 | Proteobacteria | CVUV01000236.1 |
| 72 | GCA_001180745.1 | 287 | 287 | Pseudomonas aeruginosa | P17_North_West_14_VIM_2_03_10 | Proteobacteria | CVUV01000189.1 |
| 73 | GCA_001180765.1 | 287 | 287 | Pseudomonas aeruginosa | P18_London_17_VIM_2_04_10 | Proteobacteria | CVVY01000157.1 |
| 74 | GCA_001180765.1 | 287 | 287 | Pseudomonas aeruginosa | P18_London_17_VIM_2_04_10 | Proteobacteria | CVVY01000399.1 |
| 75 | GCA_001180785.1 | 287 | 287 | Pseudomonas aeruginosa | P19_London_7_VIM_2_05_10 | Proteobacteria | CVVU01000221.1 |
| 76 | GCA_001180785.1 | 287 | 287 | Pseudomonas aeruginosa | P19_London_7_VIM_2_05_10 | Proteobacteria | CVVU01000253.1 |
| 77 | GCA_001180805.1 | 287 | 287 | Pseudomonas aeruginosa | P20_London_17_VIM_2_05_10 | Proteobacteria | CVUO01000146.1 |
| 78 | GCA_001180805.1 | 287 | 287 | Pseudomonas aeruginosa | P20_London_17_VIM_2_05_10 | Proteobacteria | CVUO01000165.1 |
| 79 | GCA_001180825.1 | 287 | 287 | Pseudomonas aeruginosa | P21_London_17_VIM_2_06_10 | Proteobacteria | CVVE01000135.1 |
| 80 | GCA_001180825.1 | 287 | 287 | Pseudomonas aeruginosa | P21_London_17_VIM_2_06_10 | Proteobacteria | CVVE01000123.1 |
| 81 | GCA_001180845.1 | 287 | 287 | Pseudomonas aeruginosa | P22_London_17_VIM_2_06_10 | Proteobacteria | CVVI01000141.1 |
| 82 | GCA_001180845.1 | 287 | 287 | Pseudomonas aeruginosa | P22_London_17_VIM_2_06_10 | Proteobacteria | CVVI01000153.1 |
| 83 | GCA_001180865.1 | 287 | 287 | Pseudomonas aeruginosa | P23_East_of_England_6_IMP_13_07_10 | Proteobacteria | CVUU01000179.1 |
| 84 | GCA_001180865.1 | 287 | 287 | Pseudomonas aeruginosa | P23_East_of_England_6_IMP_13_07_10 | Proteobacteria | CVUU01000198.1 |
| 85 | GCA_001180885.1 | 287 | 287 | Pseudomonas aeruginosa | P24_London_17_VIM_2_08_10 | Proteobacteria | CVUR01000168.1 |
| 86 | GCA_001180885.1 | 287 | 287 | Pseudomonas aeruginosa | P24_London_17_VIM_2_08_10 | Proteobacteria | CVUR01000157.1 |
| 87 | GCA_001180905.1 | 287 | 287 | Pseudomonas aeruginosa | P25_London_12_VIM_2_08_10 | Proteobacteria | CVVL01000167.1 |
| 88 | GCA_001180905.1 | 287 | 287 | Pseudomonas aeruginosa | P25_London_12_VIM_2_08_10 | Proteobacteria | CVVL01000150.1 |
| 89 | GCA_001180925.1 | 287 | 287 | Pseudomonas aeruginosa | P26_Wales_1_VIM_2_11_10 | Proteobacteria | CVVN01000216.1 |
| 90 | GCA_001180925.1 | 287 | 287 | Pseudomonas aeruginosa | P26_Wales_1_VIM_2_11_10 | Proteobacteria | CVVN01000169.1 |
| 91 | GCA_001180945.1 | 287 | 287 | Pseudomonas aeruginosa | P27_Wales_1_VIM_2_02_11 | Proteobacteria | CVVX01000242.1 |
| 92 | GCA_001180945.1 | 287 | 287 | Pseudomonas aeruginosa | P27_Wales_1_VIM_2_02_11 | Proteobacteria | CVVX01000290.1 |
| 93 | GCA_001180965.1 | 287 | 287 | Pseudomonas aeruginosa | P28_London_17_02_11 | Proteobacteria | CVUQ01000182.1 |
| 94 | GCA_001180965.1 | 287 | 287 | Pseudomonas aeruginosa | P28_London_17_02_11 | Proteobacteria | CVUQ01000192.1 |
| 95 | GCA_001180985.1 | 287 | 287 | Pseudomonas aeruginosa | P29_London_12_VIM_2_05_11 | Proteobacteria | CVVO01000189.1 |
| 96 | GCA_001180985.1 | 287 | 287 | Pseudomonas aeruginosa | P29_London_12_VIM_2_05_11 | Proteobacteria | CVVO01000175.1 |
| 97 | GCA_001181005.1 | 287 | 287 | Pseudomonas aeruginosa | P30_South_East_2_VIM_2_10_11 | Proteobacteria | CVVK01000149.1 |
| 98 | GCA_001181005.1 | 287 | 287 | Pseudomonas aeruginosa | P30_South_East_2_VIM_2_10_11 | Proteobacteria | CVVK01000161.1 |
| 99 | GCA_001181025.1 | 287 | 287 | Pseudomonas aeruginosa | P31_Wales_1_VIM_2_11_11 | Proteobacteria | CVVS01000268.1 |
| 100 | GCA_001181025.1 | 287 | 287 | Pseudomonas aeruginosa | P31_Wales_1_VIM_2_11_11 | Proteobacteria | CVVS01000311.1 |
| 101 | GCA_001181045.1 | 287 | 287 | Pseudomonas aeruginosa | P32_London_17_VIM_2_10_11 | Proteobacteria | CVVJ01000160.1 |
| 102 | GCA_001181045.1 | 287 | 287 | Pseudomonas aeruginosa | P32_London_17_VIM_2_10_11 | Proteobacteria | CVVJ01000144.1 |
| 103 | GCA_001181065.1 | 287 | 287 | Pseudomonas aeruginosa | P33_London_28_VIM_2_02_12 | Proteobacteria | CVVG01000131.1 |
| 104 | GCA_001181065.1 | 287 | 287 | Pseudomonas aeruginosa | P33_London_28_VIM_2_02_12 | Proteobacteria | CVVG01000143.1 |
| 105 | GCA_001181085.1 | 287 | 287 | Pseudomonas aeruginosa | P34_London_28_VIM_2_02_12 | Proteobacteria | CVVF01000155.1 |
| 106 | GCA_001181085.1 | 287 | 287 | Pseudomonas aeruginosa | P34_London_28_VIM_2_02_12 | Proteobacteria | CVVF01000134.1 |
| 107 | GCA_001181105.1 | 287 | 287 | Pseudomonas aeruginosa | P35_London_26_VIM_2_05_12 | Proteobacteria | CVUS01000188.1 |
| 108 | GCA_001181105.1 | 287 | 287 | Pseudomonas aeruginosa | P35_London_26_VIM_2_05_12 | Proteobacteria | CVUS01000164.1 |
| 109 | GCA_001181125.1 | 287 | 287 | Pseudomonas aeruginosa | P36_West_Midlands_5_VIM_2_06_12 | Proteobacteria | CVVP01000172.1 |
| 110 | GCA_001181125.1 | 287 | 287 | Pseudomonas aeruginosa | P36_West_Midlands_5_VIM_2_06_12 | Proteobacteria | CVVP01000215.1 |
| 111 | GCA_001181145.1 | 287 | 287 | Pseudomonas aeruginosa | P37_London_28_VIM_2_07_12 | Proteobacteria | CVUJ01000135.1 |
| 112 | GCA_001181145.1 | 287 | 287 | Pseudomonas aeruginosa | P37_London_28_VIM_2_07_12 | Proteobacteria | CVUJ01000152.1 |
| 113 | GCA_001181165.1 | 287 | 287 | Pseudomonas aeruginosa | P38_London_12_VIM_2_08_12 | Proteobacteria | CVUT01000161.1 |
| 114 | GCA_001181165.1 | 287 | 287 | Pseudomonas aeruginosa | P38_London_12_VIM_2_08_12 | Proteobacteria | CVUT01000152.1 |
| 115 | GCA_001181185.1 | 287 | 287 | Pseudomonas aeruginosa | P39_London_29_08_12 | Proteobacteria | CVVC01000121.1 |
| 116 | GCA_001181185.1 | 287 | 287 | Pseudomonas aeruginosa | P39_London_29_08_12 | Proteobacteria | CVVC01000131.1 |
| 117 | GCA_001181205.1 | 287 | 287 | Pseudomonas aeruginosa | P40_Scotland_4_VIM_2_09_12 | Proteobacteria | CVUM01000136.1 |
| 118 | GCA_001181205.1 | 287 | 287 | Pseudomonas aeruginosa | P40_Scotland_4_VIM_2_09_12 | Proteobacteria | CVUM01000124.1 |
| 119 | GCA_001181225.1 | 287 | 287 | Pseudomonas aeruginosa | P41_London_28_09_12 | Proteobacteria | CVVR01000160.1 |
| 120 | GCA_001181225.1 | 287 | 287 | Pseudomonas aeruginosa | P41_London_28_09_12 | Proteobacteria | CVVR01000143.1 |
| 121 | GCA_001181245.1 | 287 | 287 | Pseudomonas aeruginosa | P42_1_London_26_VIM_2_10_12 | Proteobacteria | CVWH01000143.1 |
| 122 | GCA_001181245.1 | 287 | 287 | Pseudomonas aeruginosa | P42_1_London_26_VIM_2_10_12 | Proteobacteria | CVWH01000149.1 |
| 123 | GCA_001181265.1 | 287 | 287 | Pseudomonas aeruginosa | P43_1_London_9_VIM_2_11_12 | Proteobacteria | CVWJ01000147.1 |
| 124 | GCA_001181265.1 | 287 | 287 | Pseudomonas aeruginosa | P43_1_London_9_VIM_2_11_12 | Proteobacteria | CVWJ01000137.1 |
| 125 | GCA_001181285.1 | 287 | 287 | Pseudomonas aeruginosa | P42_2_London_28_VIM_2_11_12 | Proteobacteria | CVWI01000128.1 |
| 126 | GCA_001181285.1 | 287 | 287 | Pseudomonas aeruginosa | P42_2_London_28_VIM_2_11_12 | Proteobacteria | CVWI01000133.1 |
| 127 | GCA_001181305.1 | 287 | 287 | Pseudomonas aeruginosa | P45_London_17_VIM_2_12_12 | Proteobacteria | CVWO01000030.1 |
| 128 | GCA_001181305.1 | 287 | 287 | Pseudomonas aeruginosa | P45_London_17_VIM_2_12_12 | Proteobacteria | CVWO01000099.1 |
| 129 | GCA_001181325.1 | 287 | 287 | Pseudomonas aeruginosa | E5_London_17_VIM_2_12_12 | Proteobacteria | CVWE01000133.1 |
| 130 | GCA_001181325.1 | 287 | 287 | Pseudomonas aeruginosa | E5_London_17_VIM_2_12_12 | Proteobacteria | CVWE01000188.1 |
| 131 | GCA_001181345.1 | 287 | 287 | Pseudomonas aeruginosa | E6_London_17_VIM_2_12_12 | Proteobacteria | CVWD01000029.1 |
| 132 | GCA_001181345.1 | 287 | 287 | Pseudomonas aeruginosa | E6_London_17_VIM_2_12_12 | Proteobacteria | CVWD01000443.1 |
| 133 | GCA_001181365.1 | 287 | 287 | Pseudomonas aeruginosa | P46_South_East_6_12_12 | Proteobacteria | CVWM01000196.1 |
| 134 | GCA_001181365.1 | 287 | 287 | Pseudomonas aeruginosa | P46_South_East_6_12_12 | Proteobacteria | CVWM01000176.1 |
| 135 | GCA_001181385.1 | 287 | 287 | Pseudomonas aeruginosa | P47_London_12_VIM_2_12_12 | Proteobacteria | CVWN01000372.1 |
| 136 | GCA_001181385.1 | 287 | 287 | Pseudomonas aeruginosa | P47_London_12_VIM_2_12_12 | Proteobacteria | CVWN01000371.1 |
| 137 | GCA_001181405.1 | 287 | 287 | Pseudomonas aeruginosa | P48_London_17_VIM_2_01_13 | Proteobacteria | CVWT01000099.1 |
| 138 | GCA_001181405.1 | 287 | 287 | Pseudomonas aeruginosa | P48_London_17_VIM_2_01_13 | Proteobacteria | CVWT01000133.1 |
| 139 | GCA_001181425.1 | 287 | 287 | Pseudomonas aeruginosa | P49_London_7_VIM_2_01_13 | Proteobacteria | CVWQ01000279.1 |
| 140 | GCA_001181425.1 | 287 | 287 | Pseudomonas aeruginosa | P49_London_7_VIM_2_01_13 | Proteobacteria | CVWQ01000286.1 |
| 141 | GCA_001181445.1 | 287 | 287 | Pseudomonas aeruginosa | P50_London_9_VIM_2_01_13 | Proteobacteria | CVWP01000147.1 |
| 142 | GCA_001181445.1 | 287 | 287 | Pseudomonas aeruginosa | P50_London_9_VIM_2_01_13 | Proteobacteria | CVWP01000136.1 |
| 143 | GCA_001181465.1 | 287 | 287 | Pseudomonas aeruginosa | P43_2_London_9_VIM_2_11_12 | Proteobacteria | CVWK01000146.1 |
| 144 | GCA_001181465.1 | 287 | 287 | Pseudomonas aeruginosa | P43_2_London_9_VIM_2_11_12 | Proteobacteria | CVWK01000139.1 |
| 145 | GCA_001181485.1 | 287 | 287 | Pseudomonas aeruginosa | P51_1_London_9_VIM_2_02_13 | Proteobacteria | CVWR01000277.1 |
| 146 | GCA_001181485.1 | 287 | 287 | Pseudomonas aeruginosa | P51_1_London_9_VIM_2_02_13 | Proteobacteria | CVWR01000249.1 |
| 147 | GCA_001181505.1 | 287 | 287 | Pseudomonas aeruginosa | E7_London_9_VIM_2_02_13 | Proteobacteria | CVWB01000104.1 |
| 148 | GCA_001181505.1 | 287 | 287 | Pseudomonas aeruginosa | E7_London_9_VIM_2_02_13 | Proteobacteria | CVWB01000325.1 |
| 149 | GCA_001181525.1 | 287 | 287 | Pseudomonas aeruginosa | P52_1_London_26_VIM_2_02_13 | Proteobacteria | CVWV01000212.1 |
| 150 | GCA_001181525.1 | 287 | 287 | Pseudomonas aeruginosa | P52_1_London_26_VIM_2_02_13 | Proteobacteria | CVWV01000231.1 |
| 151 | GCA_001181545.1 | 287 | 287 | Pseudomonas aeruginosa | P51_2_London_11_VIM_2_02_13 | Proteobacteria | CVWU01000143.1 |
| 152 | GCA_001181545.1 | 287 | 287 | Pseudomonas aeruginosa | P51_2_London_11_VIM_2_02_13 | Proteobacteria | CVWU01000157.1 |
| 153 | GCA_001181565.1 | 287 | 287 | Pseudomonas aeruginosa | P52_2_London_26_VIM_2_02_13 | Proteobacteria | CVWW01000197.1 |
| 154 | GCA_001181565.1 | 287 | 287 | Pseudomonas aeruginosa | P52_2_London_26_VIM_2_02_13 | Proteobacteria | CVWW01000223.1 |
| 155 | GCA_001181585.1 | 287 | 287 | Pseudomonas aeruginosa | P53_London_9_VIM_2_02_13 | Proteobacteria | CWER01000327.1 |
| 156 | GCA_001181585.1 | 287 | 287 | Pseudomonas aeruginosa | P53_London_9_VIM_2_02_13 | Proteobacteria | CWER01000323.1 |
| 157 | GCA_001181605.1 | 287 | 287 | Pseudomonas aeruginosa | P54_1_London_24_VIM_2_04_13 | Proteobacteria | CWFR01000366.1 |
| 158 | GCA_001181605.1 | 287 | 287 | Pseudomonas aeruginosa | P54_1_London_24_VIM_2_04_13 | Proteobacteria | CWFR01000421.1 |
| 159 | GCA_001181625.1 | 287 | 287 | Pseudomonas aeruginosa | E8_London_17_VIM_2_04_13 | Proteobacteria | CVUW01000183.1 |
| 160 | GCA_001181625.1 | 287 | 287 | Pseudomonas aeruginosa | E8_London_17_VIM_2_04_13 | Proteobacteria | CVUW01000203.1 |
| 161 | GCA_001181645.1 | 287 | 287 | Pseudomonas aeruginosa | E9_London_17_VIM_2_04_13 | Proteobacteria | CVUN01000148.1 |
| 162 | GCA_001181645.1 | 287 | 287 | Pseudomonas aeruginosa | E9_London_17_VIM_2_04_13 | Proteobacteria | CVUN01000161.1 |
| 163 | GCA_001181665.1 | 287 | 287 | Pseudomonas aeruginosa | P54_2_London_24_VIM_2_04_13 | Proteobacteria | CVXL01000226.1 |
| 164 | GCA_001181665.1 | 287 | 287 | Pseudomonas aeruginosa | P54_2_London_24_VIM_2_04_13 | Proteobacteria | CVXL01000242.1 |
| 165 | GCA_001181685.1 | 287 | 287 | Pseudomonas aeruginosa | P55_London_26_VIM_2_05_13 | Proteobacteria | CVWX01000125.1 |
| 166 | GCA_001181685.1 | 287 | 287 | Pseudomonas aeruginosa | P55_London_26_VIM_2_05_13 | Proteobacteria | CVWX01000140.1 |
| 167 | GCA_001181705.1 | 287 | 287 | Pseudomonas aeruginosa | E10_London_26_VIM_2_06_13 | Proteobacteria | CVVZ01000308.1 |
| 168 | GCA_001181705.1 | 287 | 287 | Pseudomonas aeruginosa | E10_London_26_VIM_2_06_13 | Proteobacteria | CVVZ01000025.1 |
| 169 | GCA_001181725.1 | 287 | 287 | Pseudomonas aeruginosa | E11_London_26_VIM_2_06_13 | Proteobacteria | CVVM01000142.1 |
| 170 | GCA_001181725.1 | 287 | 287 | Pseudomonas aeruginosa | E11_London_26_VIM_2_06_13 | Proteobacteria | CVVM01000156.1 |
| 171 | GCA_001181745.1 | 287 | 287 | Pseudomonas aeruginosa | E12_London_26_VIM_2_06_13 | Proteobacteria | CVUX01000216.1 |
| 172 | GCA_001181745.1 | 287 | 287 | Pseudomonas aeruginosa | E12_London_26_VIM_2_06_13 | Proteobacteria | CVUX01000199.1 |
| 173 | GCA_001181765.1 | 287 | 287 | Pseudomonas aeruginosa | E13_London_26_VIM_2_06_13 | Proteobacteria | CVVQ01000190.1 |
| 174 | GCA_001181765.1 | 287 | 287 | Pseudomonas aeruginosa | E13_London_26_VIM_2_06_13 | Proteobacteria | CVVQ01000177.1 |
| 175 | GCA_001181785.1 | 287 | 287 | Pseudomonas aeruginosa | E14_London_26_VIM_2_06_13 | Proteobacteria | CWGG01000138.1 |
| 176 | GCA_001181785.1 | 287 | 287 | Pseudomonas aeruginosa | E14_London_26_VIM_2_06_13 | Proteobacteria | CWGG01000153.1 |
| 177 | GCA_001181805.1 | 287 | 287 | Pseudomonas aeruginosa | P56_London_12_VIM_2_07_13 | Proteobacteria | CVXC01000168.1 |
| 178 | GCA_001181805.1 | 287 | 287 | Pseudomonas aeruginosa | P56_London_12_VIM_2_07_13 | Proteobacteria | CVXC01000184.1 |
| 179 | GCA_001181825.1 | 287 | 287 | Pseudomonas aeruginosa | P8_2_London_17_VIM_2_07_13 | Proteobacteria | CVXH01000145.1 |
| 180 | GCA_001181825.1 | 287 | 287 | Pseudomonas aeruginosa | P8_2_London_17_VIM_2_07_13 | Proteobacteria | CVXH01000148.1 |
| 181 | GCA_001181845.1 | 287 | 287 | Pseudomonas aeruginosa | P57_South_East_3_VIM_2_09_13 | Proteobacteria | CVWZ01000144.1 |
| 182 | GCA_001181845.1 | 287 | 287 | Pseudomonas aeruginosa | P57_South_East_3_VIM_2_09_13 | Proteobacteria | CVWZ01000159.1 |
| 183 | GCA_001181865.1 | 287 | 287 | Pseudomonas aeruginosa | P58_London_29_09_13 | Proteobacteria | CVXA01000139.1 |
| 184 | GCA_001181865.1 | 287 | 287 | Pseudomonas aeruginosa | P58_London_29_09_13 | Proteobacteria | CVXA01000156.1 |
| 185 | GCA_001181885.1 | 287 | 287 | Pseudomonas aeruginosa | P59_Wales_1_VIM_2_09_13 | Proteobacteria | CVXN01000001.1 |
| 186 | GCA_001181885.1 | 287 | 287 | Pseudomonas aeruginosa | P59_Wales_1_VIM_2_09_13 | Proteobacteria | CVXN01000252.1 |
| 187 | GCA_001181905.1 | 287 | 287 | Pseudomonas aeruginosa | P60_London_6_VIM_2_11_13 | Proteobacteria | CVXD01000175.1 |
| 188 | GCA_001181905.1 | 287 | 287 | Pseudomonas aeruginosa | P60_London_6_VIM_2_11_13 | Proteobacteria | CVXD01000153.1 |
| 189 | GCA_001181925.1 | 287 | 287 | Pseudomonas aeruginosa | P61_London_9_VIM_2_11_13 | Proteobacteria | CVXB01000157.1 |
| 190 | GCA_001181925.1 | 287 | 287 | Pseudomonas aeruginosa | P61_London_9_VIM_2_11_13 | Proteobacteria | CVXB01000171.1 |
| 191 | GCA_001181945.1 | 287 | 287 | Pseudomonas aeruginosa | P64_East_of_England_6_01_14 | Proteobacteria | CWFQ01000009.1 |
| 192 | GCA_001181945.1 | 287 | 287 | Pseudomonas aeruginosa | P64_East_of_England_6_01_14 | Proteobacteria | CWFQ01000391.1 |
| 193 | GCA_001181965.1 | 287 | 287 | Pseudomonas aeruginosa | P62_London_9_VIM_2_01_14 | Proteobacteria | CWEO01000265.1 |
| 194 | GCA_001181965.1 | 287 | 287 | Pseudomonas aeruginosa | P62_London_9_VIM_2_01_14 | Proteobacteria | CWEO01000250.1 |
| 195 | GCA_001181985.1 | 287 | 287 | Pseudomonas aeruginosa | E15_London_28_01_14 | Proteobacteria | CVWF01000732.1 |
| 196 | GCA_001182005.1 | 287 | 287 | Pseudomonas aeruginosa | P63_London_25_VIM_2_03_14 | Proteobacteria | CWFH01000250.1 |
| 197 | GCA_001182005.1 | 287 | 287 | Pseudomonas aeruginosa | P63_London_25_VIM_2_03_14 | Proteobacteria | CWFH01000268.1 |
| 198 | GCA_001182025.1 | 287 | 287 | Pseudomonas aeruginosa | E16_London_17_VIM_2_04_14 | Proteobacteria | CVWG01001003.1 |
| 199 | GCA_001182025.1 | 287 | 287 | Pseudomonas aeruginosa | E16_London_17_VIM_2_04_14 | Proteobacteria | CVWG01000111.1 |
| 200 | GCA_001182945.1 | 287 | 287 | Pseudomonas aeruginosa | P44_Wales_1_VIM_2_11_12 | Proteobacteria | CVWL01000374.1 |
| 201 | GCA_001182945.1 | 287 | 287 | Pseudomonas aeruginosa | P44_Wales_1_VIM_2_11_12 | Proteobacteria | CVWL01000327.1 |
| 202 | GCA_001420185.1 | 287 | 287 | Pseudomonas aeruginosa | ATCC 33349 | Proteobacteria | LINV01000076.1 |
| 203 | GCA_001444845.1 | 287 | 287 | Pseudomonas aeruginosa | ATCC 33361 | Proteobacteria | LIZH01000277.1 |
| 204 | GCA_001444865.1 | 287 | 287 | Pseudomonas aeruginosa | ATCC 33362 | Proteobacteria | LIZI01000121.1 |
| 205 | GCA_001444975.1 | 287 | 287 | Pseudomonas aeruginosa | ATCC 33359 | Proteobacteria | LIZF01000253.1 |
| 206 | GCA_001444995.1 | 287 | 287 | Pseudomonas aeruginosa | ATCC 33360 | Proteobacteria | LIZG01000273.1 |
| 207 | GCA_001449385.1 | 287 | 287 | Pseudomonas aeruginosa | WH-SGI-V-07072 | Proteobacteria | LLLP01000007.1 |
| 208 | GCA_001449465.1 | 287 | 287 | Pseudomonas aeruginosa | WH-SGI-V-07165 | Proteobacteria | LLLU01000037.1 |
| 209 | GCA_001449505.1 | 287 | 287 | Pseudomonas aeruginosa | WH-SGI-V-07167 | Proteobacteria | LLLW01000014.1 |
| 210 | GCA_001449515.1 | 287 | 287 | Pseudomonas aeruginosa | WH-SGI-V-07168 | Proteobacteria | LLLX01000036.1 |
| 211 | GCA_001449515.1 | 287 | 287 | Pseudomonas aeruginosa | WH-SGI-V-07168 | Proteobacteria | LLLX01000030.1 |
| 212 | GCA_001449555.1 | 287 | 287 | Pseudomonas aeruginosa | WH-SGI-V-07171 | Proteobacteria | LLMA01000028.1 |
| 213 | GCA_001449555.1 | 287 | 287 | Pseudomonas aeruginosa | WH-SGI-V-07171 | Proteobacteria | LLMA01000003.1 |
| 214 | GCA_001449605.1 | 287 | 287 | Pseudomonas aeruginosa | WH-SGI-V-07174 | Proteobacteria | LLMD01000001.1 |
| 215 | GCA_001449605.1 | 287 | 287 | Pseudomonas aeruginosa | WH-SGI-V-07174 | Proteobacteria | LLMD01000070.1 |
| 216 | GCA_001449625.1 | 287 | 287 | Pseudomonas aeruginosa | WH-SGI-V-07175 | Proteobacteria | LLME01000030.1 |
| 217 | GCA_001449625.1 | 287 | 287 | Pseudomonas aeruginosa | WH-SGI-V-07175 | Proteobacteria | LLME01000022.1 |
| 218 | GCA_001449675.1 | 287 | 287 | Pseudomonas aeruginosa | WH-SGI-V-07180 | Proteobacteria | LLMJ01000031.1 |
| 219 | GCA_001449705.1 | 287 | 287 | Pseudomonas aeruginosa | WH-SGI-V-07221 | Proteobacteria | LLMK01000076.1 |
| 220 | GCA_001449705.1 | 287 | 287 | Pseudomonas aeruginosa | WH-SGI-V-07221 | Proteobacteria | LLMK01000071.1 |
| 221 | GCA_001450165.1 | 287 | 287 | Pseudomonas aeruginosa | WH-SGI-V-07370 | Proteobacteria | LLNU01000057.1 |
| 222 | GCA_001450275.1 | 287 | 287 | Pseudomonas aeruginosa | WH-SGI-V-07379 | Proteobacteria | LLOD01000041.1 |
| 223 | GCA_001450355.1 | 287 | 287 | Pseudomonas aeruginosa | WH-SGI-V-07055 | Proteobacteria | LLKZ01000040.1 |
| 224 | GCA_001450385.1 | 287 | 287 | Pseudomonas aeruginosa | WH-SGI-V-07065 | Proteobacteria | LLLC01000012.1 |
| 225 | GCA_001450465.1 | 287 | 287 | Pseudomonas aeruginosa | WH-SGI-V-07064 | Proteobacteria | LLLN01000006.1 |
| 226 | GCA_001450525.1 | 287 | 287 | Pseudomonas aeruginosa | WH-SGI-V-07173 | Proteobacteria | LLMC01000045.1 |
| 227 | GCA_001450525.1 | 287 | 287 | Pseudomonas aeruginosa | WH-SGI-V-07173 | Proteobacteria | LLMC01000054.1 |
| 228 | GCA_001450625.1 | 287 | 287 | Pseudomonas aeruginosa | WH-SGI-V-07185 | Proteobacteria | LLMP01000039.1 |
| 229 | GCA_001451065.1 | 287 | 287 | Pseudomonas aeruginosa | WH-SGI-V-07392 | Proteobacteria | LLOQ01000102.1 |
| 230 | GCA_001451075.1 | 287 | 287 | Pseudomonas aeruginosa | WH-SGI-V-07391 | Proteobacteria | LLOP01000052.1 |
| 231 | GCA_001451225.1 | 287 | 287 | Pseudomonas aeruginosa | WH-SGI-V-07415 | Proteobacteria | LLPC01000038.1 |
| 232 | GCA_001451505.1 | 287 | 287 | Pseudomonas aeruginosa | WH-SGI-V-07426 | Proteobacteria | LLPN01000122.1 |
| 233 | GCA_001451705.1 | 287 | 287 | Pseudomonas aeruginosa | WH-SGI-V-07491 | Proteobacteria | LLPX01000023.1 |
| 234 | GCA_001451805.1 | 287 | 287 | Pseudomonas aeruginosa | WH-SGI-V-07497 | Proteobacteria | LLQD01000079.1 |
| 235 | GCA_001451945.1 | 287 | 287 | Pseudomonas aeruginosa | WH-SGI-V-07618 | Proteobacteria | LLQP01000102.1 |
| 236 | GCA_001452185.1 | 287 | 287 | Pseudomonas aeruginosa | WH-SGI-V-07637 | Proteobacteria | LLRI01000005.1 |
| 237 | GCA_001452205.1 | 287 | 287 | Pseudomonas aeruginosa | WH-SGI-V-07640 | Proteobacteria | LLRL01000163.1 |
| 238 | GCA_001452365.1 | 287 | 287 | Pseudomonas aeruginosa | WH-SGI-V-07651 | Proteobacteria | LLRW01000066.1 |
| 239 | GCA_001452365.1 | 287 | 287 | Pseudomonas aeruginosa | WH-SGI-V-07651 | Proteobacteria | LLRW01000061.1 |
| 240 | GCA_001452385.1 | 287 | 287 | Pseudomonas aeruginosa | WH-SGI-V-07652 | Proteobacteria | LLRX01000045.1 |
| 241 | GCA_001452415.1 | 287 | 287 | Pseudomonas aeruginosa | WH-SGI-V-07681 | Proteobacteria | LLSB01000007.1 |
| 242 | GCA_001452465.1 | 287 | 287 | Pseudomonas aeruginosa | WH-SGI-V-07687 | Proteobacteria | LLSH01000054.1 |
| 243 | GCA_001452485.1 | 287 | 287 | Pseudomonas aeruginosa | WH-SGI-V-07688 | Proteobacteria | LLSI01000049.1 |
| 244 | GCA_001452485.1 | 287 | 287 | Pseudomonas aeruginosa | WH-SGI-V-07688 | Proteobacteria | LLSI01000001.1 |
| 245 | GCA_001452565.1 | 287 | 287 | Pseudomonas aeruginosa | WH-SGI-V-07693 | Proteobacteria | LLSN01000046.1 |
| 246 | GCA_001452645.1 | 287 | 287 | Pseudomonas aeruginosa | WH-SGI-V-07700 | Proteobacteria | LLSU01000053.1 |
| 247 | GCA_001452645.1 | 287 | 287 | Pseudomonas aeruginosa | WH-SGI-V-07700 | Proteobacteria | LLSU01000046.1 |
| 248 | GCA_001452665.1 | 287 | 287 | Pseudomonas aeruginosa | WH-SGI-V-07702 | Proteobacteria | LLSW01000054.1 |
| 249 | GCA_001452665.1 | 287 | 287 | Pseudomonas aeruginosa | WH-SGI-V-07702 | Proteobacteria | LLSW01000062.1 |
| 250 | GCA_001452765.1 | 287 | 287 | Pseudomonas aeruginosa | WH-SGI-V-07711 | Proteobacteria | LLTF01000239.1 |
| 251 | GCA_001452905.1 | 287 | 287 | Pseudomonas aeruginosa | WH-SGI-V-07253 | Proteobacteria | LLTN01000034.1 |
| 252 | GCA_001453015.1 | 287 | 287 | Pseudomonas aeruginosa | WH-SGI-V-07263 | Proteobacteria | LLTV01000056.1 |
| 253 | GCA_001453615.1 | 287 | 287 | Pseudomonas aeruginosa | WH-SGI-V-07684 | Proteobacteria | LLSE01000089.1 |
| 254 | GCA_001453755.1 | 287 | 287 | Pseudomonas aeruginosa | WH-SGI-V-07705 | Proteobacteria | LLSZ01000120.1 |
| 255 | GCA_001453855.1 | 287 | 287 | Pseudomonas aeruginosa | WH-SGI-V-07256 | Proteobacteria | LLTQ01000112.1 |
| 256 | GCA_001453855.1 | 287 | 287 | Pseudomonas aeruginosa | WH-SGI-V-07256 | Proteobacteria | LLTQ01000118.1 |
| 257 | GCA_001453885.1 | 287 | 287 | Pseudomonas aeruginosa | WH-SGI-V-07259 | Proteobacteria | LLTS01000104.1 |
| 258 | GCA_001453885.1 | 287 | 287 | Pseudomonas aeruginosa | WH-SGI-V-07259 | Proteobacteria | LLTS01000112.1 |
| 259 | GCA_001454125.1 | 287 | 287 | Pseudomonas aeruginosa | WH-SGI-V-07290 | Proteobacteria | LLUN01000012.1 |
| 260 | GCA_001454145.1 | 287 | 287 | Pseudomonas aeruginosa | WH-SGI-V-07291 | Proteobacteria | LLUO01000109.1 |
| 261 | GCA_001454205.1 | 287 | 287 | Pseudomonas aeruginosa | WH-SGI-V-07295 | Proteobacteria | LLUR01000002.1 |
| 262 | GCA_001454225.1 | 287 | 287 | Pseudomonas aeruginosa | WH-SGI-V-07296 | Proteobacteria | LLUS01000044.1 |
| 263 | GCA_001454225.1 | 287 | 287 | Pseudomonas aeruginosa | WH-SGI-V-07296 | Proteobacteria | LLUS01000001.1 |
| 264 | GCA_001454445.1 | 287 | 287 | Pseudomonas aeruginosa | WH-SGI-V-07322 | Proteobacteria | LLVI01000081.1 |
| 265 | GCA_001454445.1 | 287 | 287 | Pseudomonas aeruginosa | WH-SGI-V-07322 | Proteobacteria | LLVI01000088.1 |
| 266 | GCA_001454485.1 | 287 | 287 | Pseudomonas aeruginosa | WH-SGI-V-07324 | Proteobacteria | LLVK01000064.1 |
| 267 | GCA_001454485.1 | 287 | 287 | Pseudomonas aeruginosa | WH-SGI-V-07324 | Proteobacteria | LLVK01000044.1 |
| 268 | GCA_001454495.1 | 287 | 287 | Pseudomonas aeruginosa | WH-SGI-V-07325 | Proteobacteria | LLVL01000090.1 |
| 269 | GCA_001454495.1 | 287 | 287 | Pseudomonas aeruginosa | WH-SGI-V-07325 | Proteobacteria | LLVL01000001.1 |
| 270 | GCA_001454515.1 | 287 | 287 | Pseudomonas aeruginosa | WH-SGI-V-07328 | Proteobacteria | LLVO01000057.1 |
| 271 | GCA_001454515.1 | 287 | 287 | Pseudomonas aeruginosa | WH-SGI-V-07328 | Proteobacteria | LLVO01000082.1 |
| 272 | GCA_001454545.1 | 287 | 287 | Pseudomonas aeruginosa | WH-SGI-V-07329 | Proteobacteria | LLVP01000031.1 |
| 273 | GCA_001454545.1 | 287 | 287 | Pseudomonas aeruginosa | WH-SGI-V-07329 | Proteobacteria | LLVP01000037.1 |
| 274 | GCA_001454565.1 | 287 | 287 | Pseudomonas aeruginosa | WH-SGI-V-07330 | Proteobacteria | LLVQ01000079.1 |
| 275 | GCA_001454565.1 | 287 | 287 | Pseudomonas aeruginosa | WH-SGI-V-07330 | Proteobacteria | LLVQ01000086.1 |
| 276 | GCA_001454675.1 | 287 | 287 | Pseudomonas aeruginosa | WH-SGI-V-07287 | Proteobacteria | LLUM01000045.1 |
| 277 | GCA_001454805.1 | 287 | 287 | Pseudomonas aeruginosa | WH-SGI-V-07326 | Proteobacteria | LLVM01000239.1 |
| 278 | GCA_001454805.1 | 287 | 287 | Pseudomonas aeruginosa | WH-SGI-V-07326 | Proteobacteria | LLVM01000224.1 |
| 279 | GCA_001454825.1 | 287 | 287 | Pseudomonas aeruginosa | WH-SGI-V-07327 | Proteobacteria | LLVN01000114.1 |
| 280 | GCA_001454825.1 | 287 | 287 | Pseudomonas aeruginosa | WH-SGI-V-07327 | Proteobacteria | LLVN01000121.1 |
| 281 | GCA_001469695.1 | 287 | 287 | Pseudomonas aeruginosa | AATYA | Proteobacteria | LODN01000001.1 |
| 282 | GCA_001469695.1 | 287 | 287 | Pseudomonas aeruginosa | AATYA | Proteobacteria | LODN01000079.1 |
| 283 | GCA_001471355.1 | 287 | 287 | Pseudomonas aeruginosa | FDAARGOS_154 | Proteobacteria | LORM01000001.1 |
| 284 | GCA_001516165.1 | 287 | 287 | Pseudomonas aeruginosa | W16407 | Proteobacteria | CP008869.1 |
| 285 | GCA_001516245.1 | 287 | 287 | Pseudomonas aeruginosa | F23197 | Proteobacteria | CP008856.1 |
| 286 | GCA_001516265.1 | 287 | 287 | Pseudomonas aeruginosa | F30658 | Proteobacteria | CP008857.1 |
| 287 | GCA_001516265.1 | 287 | 287 | Pseudomonas aeruginosa | F30658 | Proteobacteria | CP008857.1 |
| 288 | GCA_001535675.1 | 287 | 287 | Pseudomonas aeruginosa | SPA01 | Proteobacteria | LQBU01000001.1 |
| 289 | GCA_001547575.1 | 287 | 287 | Pseudomonas aeruginosa | BH6 | Proteobacteria | LGVH01000140.1 |
| 290 | GCA_001560865.1 | 287 | 287 | Pseudomonas aeruginosa | 105777 | Proteobacteria | LODH01000011.1 |
| 291 | GCA_001601745.1 | 287 | 287 | Pseudomonas aeruginosa | 105819 | Proteobacteria | LOHH01000029.1 |
| 292 | GCA_000877315.1 | 292 | 292 | Burkholderia cepacia | LMG 16656 | Proteobacteria | JTDP01000004.1 |
| 293 | GCA_001522525.1 | 292 | 292 | Burkholderia cepacia | INT1-BP277 | Proteobacteria | LOTX01000037.1 |
| 294 | GCA_001523085.1 | 292 | 292 | Burkholderia cepacia | INT25-BP2 | Proteobacteria | LOUT01000007.1 |
| 295 | GCA_000237065.1 | 1114970 | 294 | Pseudomonas fluorescens F113 | F113 | Proteobacteria | CP003150.1 |
| 296 | GCA_000411675.1 | 1324332 | 294 | Pseudomonas fluorescens LMG 5329 | LMG 5329 | Proteobacteria | ASGY01000250.1 |
| 297 | GCA_001542695.1 | 294 | 294 | Pseudomonas fluorescens | Ps_20 | Proteobacteria | LCYD01000004.1 |
| 298 | GCA_000016865.1 | 351746 | 303 | Pseudomonas putida F1 | F1 | Proteobacteria | CP000712.1 |
| 299 | GCA_000264665.1 | 231023 | 303 | Pseudomonas putida ND6 | ND6 | Proteobacteria | CP003588.1 |
| 300 | GCA_000294445.2 | 1193146 | 303 | Pseudomonas putida LS46 | LS46 | Proteobacteria | ALPV02000008.1 |
| 301 | GCA_000325725.1 | 1215088 | 303 | Pseudomonas putida HB3267 | PC9 | Proteobacteria | CP003738.1 |
| 302 | GCA_001467305.1 | 303 | 303 | Pseudomonas putida | ABAC8 | Proteobacteria | LKGZ01000002.1 |
| 303 | GCA_000013785.1 | 379731 | 316 | Pseudomonas stutzeri A1501 | A1501 | Proteobacteria | CP000304.1 |
| 304 | GCA_000195105.1 | 996285 | 316 | Pseudomonas stutzeri DSM 4166 | DSM 4166 | Proteobacteria | CP002622.1 |
| 305 | GCA_000237885.2 | 32042 | 316 | Pseudomonas stutzeri ATCC 14405 = CCUG 16156 | CCUG16156 | Proteobacteria | AGSL01000126.1 |
| 306 | GCA_000267545.1 | 1196835 | 316 | Pseudomonas stutzeri CCUG 29243 | CCUG 29243 | Proteobacteria | CP003677.1 |
| 307 | GCA_000307775.2 | 1218352 | 316 | Pseudomonas stutzeri KOS6 | KOS6 | Proteobacteria | KK020678.1 |
| 308 | GCA_000416345.1 | 1212549 | 316 | Pseudomonas stutzeri B1SMN1 | B1SMN1 | Proteobacteria | AMVM01000007.1 |
| 309 | GCA_000416545.1 | 1194422 | 317 | Pseudomonas syringae pv. actinidiae ICMP 19098 | ICMP 19098 | Proteobacteria | AOKE01000198.1 |
| 310 | GCA_000416645.2 | 1194405 | 317 | Pseudomonas syringae pv. actinidiae ICMP 19096 | ICMP 19096 | Proteobacteria | AOKF01004024.1 |
| 311 | GCA_000416745.1 | 1194401 | 317 | Pseudomonas syringae pv. actinidiae ICMP 19094 | ICMP 19094 | Proteobacteria | AOKJ01000458.1 |
| 312 | GCA_000416765.1 | 1194403 | 317 | Pseudomonas syringae pv. actinidiae ICMP 19095 | ICMP 19095 | Proteobacteria | AOKI01000655.1 |
| 313 | GCA_000416785.2 | 1194402 | 317 | Pseudomonas syringae pv. actinidiae ICMP 18883 | ICMP 18883 | Proteobacteria | AOKH01000282.1 |
| 314 | GCA_000416805.1 | 1194421 | 317 | Pseudomonas syringae pv. actinidiae ICMP 19099 | ICMP 19099 | Proteobacteria | AOKD01000288.1 |
| 315 | GCA_000416825.2 | 1194420 | 317 | Pseudomonas syringae pv. actinidiae ICMP 19100 | ICMP 19100 | Proteobacteria | AOKC01000529.1 |
| 316 | GCA_000416905.2 | 1104682 | 317 | Pseudomonas syringae pv. actinidiae ICMP 18804 | ICMP 18804 | Proteobacteria | AOJU01004126.1 |
| 317 | GCA_000737235.1 | 317 | 317 | Pseudomonas syringae | CEB003 | Proteobacteria | JPQT01000163.1 |
| 318 | GCA_001467225.1 | 1208099 | 317 | Pseudomonas syringae pv. actinidiae ICMP 19497 | ICMP 19497 | Proteobacteria | LKBQ01000046.1 |
| 319 | GCA_000007145.1 | 190485 | 339 | Xanthomonas campestris pv. campestris str. ATCC 33913 | ATCC 33913 | Proteobacteria | AE008922.1 |
| 320 | GCA_000012105.1 | 314565 | 339 | Xanthomonas campestris pv. campestris str. 8004 | 8004 | Proteobacteria | CP000050.1 |
| 321 | GCA_001186415.1 | 340 | 339 | Xanthomonas campestris pv. campestris | ICMP 21080 | Proteobacteria | CP012145.1 |
| 322 | GCA_000018545.1 | 394 | 380 | Sinorhizobium fredii NGR234 | NGR234 | Proteobacteria | CP001389.1 |
| 323 | GCA_001510895.1 | 381 | 381 | Mesorhizobium loti | UFLA 01-765 | Proteobacteria | LPWA01000115.1 |
| 324 | GCA_001510895.1 | 381 | 381 | Mesorhizobium loti | UFLA 01-765 | Proteobacteria | LPWA01000098.1 |
| 325 | GCA_000985895.1 | 323655 | 399 | Neorhizobium galegae bv. orientalis | | Proteobacteria | CCRN01000010.1 |
| 326 | GCA_000021845.1 | 440085 | 408 | Methylobacterium extorquens CM4 | CM4 | Proteobacteria | CP001298.1 |
| 327 | GCA_000048665.1 | 297245 | 446 | Legionella pneumophila str. Lens | Lens | Proteobacteria | CR628337.1 |
| 328 | GCA_000306865.1 | 91891 | 446 | Legionella pneumophila subsp. pneumophila | Lorraine | Proteobacteria | FQ958210.1 |
| 329 | GCA_900048955.1 | 446 | 446 | Legionella pneumophila | 2532STDY5467593 | Proteobacteria | FJID01000003.1 |
| 330 | GCA_900048965.1 | 446 | 446 | Legionella pneumophila | 2532STDY5467557 | Proteobacteria | FJJD01000021.1 |
| 331 | GCA_900049285.1 | 446 | 446 | Legionella pneumophila | 2532STDY5467623 | Proteobacteria | FJLD01000023.1 |
| 332 | GCA_900049295.1 | 446 | 446 | Legionella pneumophila | 2532STDY5467622 | Proteobacteria | FJLF01000024.1 |
| 333 | GCA_900049305.1 | 446 | 446 | Legionella pneumophila | 2532STDY5467621 | Proteobacteria | FJLH01000015.1 |
| 334 | GCA_900049375.1 | 446 | 446 | Legionella pneumophila | 2532STDY5467550 | Proteobacteria | FJJA01000022.1 |
| 335 | GCA_900050245.1 | 446 | 446 | Legionella pneumophila | 2532STDY5467555 | Proteobacteria | FJJC01000012.1 |
| 336 | GCA_900051005.1 | 446 | 446 | Legionella pneumophila | 2532STDY5467663 | Proteobacteria | FJLJ01000022.1 |
| 337 | GCA_900051015.1 | 446 | 446 | Legionella pneumophila | 2531STDY5467451 | Proteobacteria | FJLL01000023.1 |
| 338 | GCA_900051745.1 | 446 | 446 | Legionella pneumophila | 2532STDY5467554 | Proteobacteria | FJJB01000022.1 |
| 339 | GCA_900052325.1 | 446 | 446 | Legionella pneumophila | 2532STDY5467604 | Proteobacteria | FJKF01000025.1 |
| 340 | GCA_900052335.1 | 446 | 446 | Legionella pneumophila | 2532STDY5467588 | Proteobacteria | FJKM01000021.1 |
| 341 | GCA_900052945.1 | 446 | 446 | Legionella pneumophila | 2532STDY5467560 | Proteobacteria | FJIL01000023.1 |
| 342 | GCA_900053425.1 | 446 | 446 | Legionella pneumophila | 2532STDY5467573 | Proteobacteria | FJJV01000023.1 |
| 343 | GCA_900053435.1 | 446 | 446 | Legionella pneumophila | 2532STDY5467568 | Proteobacteria | FJJX01000003.1 |
| 344 | GCA_900053445.1 | 446 | 446 | Legionella pneumophila | 2532STDY5467595 | Proteobacteria | FJJZ01000021.1 |
| 345 | GCA_900054165.1 | 446 | 446 | Legionella pneumophila | 2532STDY5467626 | Proteobacteria | FJHY01000004.1 |
| 346 | GCA_900054385.1 | 446 | 446 | Legionella pneumophila | 2531STDY5467312 | Proteobacteria | FJAT01000023.1 |
| 347 | GCA_900054395.1 | 446 | 446 | Legionella pneumophila | 2531STDY5467310 | Proteobacteria | FJBR01000001.1 |
| 348 | GCA_900054645.1 | 446 | 446 | Legionella pneumophila | 2532STDY5467583 | Proteobacteria | FJKP01000024.1 |
| 349 | GCA_900054655.1 | 446 | 446 | Legionella pneumophila | 2532STDY5467613 | Proteobacteria | FJKY01000028.1 |
| 350 | GCA_900054665.1 | 446 | 446 | Legionella pneumophila | 2532STDY5467610 | Proteobacteria | FJLB01000023.1 |
| 351 | GCA_900054675.1 | 446 | 446 | Legionella pneumophila | 2532STDY5467611 | Proteobacteria | FJLA01000022.1 |
| 352 | GCA_900054685.1 | 446 | 446 | Legionella pneumophila | 2532STDY5467619 | Proteobacteria | FJLG01000023.1 |
| 353 | GCA_900055215.1 | 446 | 446 | Legionella pneumophila | 2532STDY5467582 | Proteobacteria | FJKK01000023.1 |
| 354 | GCA_900055605.1 | 446 | 446 | Legionella pneumophila | 2532STDY5467580 | Proteobacteria | FJIF01000024.1 |
| 355 | GCA_900055615.1 | 446 | 446 | Legionella pneumophila | 2532STDY5467553 | Proteobacteria | FJIH01000023.1 |
| 356 | GCA_900055625.1 | 446 | 446 | Legionella pneumophila | 2532STDY5467571 | Proteobacteria | FJJY01000024.1 |
| 357 | GCA_900055635.1 | 446 | 446 | Legionella pneumophila | 2532STDY5467607 | Proteobacteria | FJLC01000024.1 |
| 358 | GCA_900056195.1 | 446 | 446 | Legionella pneumophila | 2532STDY5467551 | Proteobacteria | FJJP01000022.1 |
| 359 | GCA_900056205.1 | 446 | 446 | Legionella pneumophila | 2532STDY5467617 | Proteobacteria | FJKS01000024.1 |
| 360 | GCA_900056215.1 | 446 | 446 | Legionella pneumophila | 2532STDY5467616 | Proteobacteria | FJKT01000023.1 |
| 361 | GCA_900056935.1 | 446 | 446 | Legionella pneumophila | 2532STDY5467541 | Proteobacteria | FJJR01000023.1 |
| 362 | GCA_900056945.1 | 446 | 446 | Legionella pneumophila | 2532STDY5467540 | Proteobacteria | FJJU01000024.1 |
| 363 | GCA_900057265.1 | 446 | 446 | Legionella pneumophila | 2532STDY5467592 | Proteobacteria | FJKR01000024.1 |
| 364 | GCA_900057575.1 | 446 | 446 | Legionella pneumophila | 2532STDY5467547 | Proteobacteria | FJIP01000023.1 |
| 365 | GCA_900057585.1 | 446 | 446 | Legionella pneumophila | 2532STDY5467543 | Proteobacteria | FJJT01000023.1 |
| 366 | GCA_900057595.1 | 446 | 446 | Legionella pneumophila | 2532STDY5467574 | Proteobacteria | FJKC01000003.1 |
| 367 | GCA_900057605.1 | 446 | 446 | Legionella pneumophila | 2532STDY5467625 | Proteobacteria | FJKU01000024.1 |
| 368 | GCA_900057615.1 | 446 | 446 | Legionella pneumophila | 2531STDY5467449 | Proteobacteria | FJLM01000024.1 |
| 369 | GCA_900057785.1 | 446 | 446 | Legionella pneumophila | 2532STDY5467576 | Proteobacteria | FJIA01000024.1 |
| 370 | GCA_900058605.1 | 446 | 446 | Legionella pneumophila | 2532STDY5467563 | Proteobacteria | FJJL01000023.1 |
| 371 | GCA_900058825.1 | 446 | 446 | Legionella pneumophila | 2532STDY5467600 | Proteobacteria | FJIT01000024.1 |
| 372 | GCA_900058835.1 | 446 | 446 | Legionella pneumophila | 2532STDY5467548 | Proteobacteria | FJIZ01000023.1 |
| 373 | GCA_900059595.1 | 446 | 446 | Legionella pneumophila | 2532STDY5467603 | Proteobacteria | FJKE01000023.1 |
| 374 | GCA_900059605.1 | 446 | 446 | Legionella pneumophila | 2532STDY5467614 | Proteobacteria | FJKV01000024.1 |
| 375 | GCA_900060005.1 | 446 | 446 | Legionella pneumophila | 2532STDY5467535 | Proteobacteria | FJIG01000023.1 |
| 376 | GCA_900060015.1 | 446 | 446 | Legionella pneumophila | 2532STDY5467596 | Proteobacteria | FJJG01000023.1 |
| 377 | GCA_900060415.1 | 446 | 446 | Legionella pneumophila | 2532STDY5467542 | Proteobacteria | FJII01000024.1 |
| 378 | GCA_900060425.1 | 446 | 446 | Legionella pneumophila | 2532STDY5467549 | Proteobacteria | FJIJ01000024.1 |
| 379 | GCA_900060435.1 | 446 | 446 | Legionella pneumophila | 2532STDY5467544 | Proteobacteria | FJIK01000023.1 |
| 380 | GCA_900060445.1 | 446 | 446 | Legionella pneumophila | 2532STDY5467537 | Proteobacteria | FJIM01000023.1 |
| 381 | GCA_900060455.1 | 446 | 446 | Legionella pneumophila | 2532STDY5467584 | Proteobacteria | FJKQ01000023.1 |
| 382 | GCA_900060775.1 | 446 | 446 | Legionella pneumophila | 2532STDY5467627 | Proteobacteria | FJIS01000025.1 |
| 383 | GCA_900060785.1 | 446 | 446 | Legionella pneumophila | 2532STDY5467556 | Proteobacteria | FJJF01000024.1 |
| 384 | GCA_900061535.1 | 446 | 446 | Legionella pneumophila | 2531STDY5467351 | Proteobacteria | FJCL01000024.1 |
| 385 | GCA_900062525.1 | 446 | 446 | Legionella pneumophila | 2531STDY5467450 | Proteobacteria | FJHW01000004.1 |
| 386 | GCA_900062535.1 | 446 | 446 | Legionella pneumophila | 2532STDY5467575 | Proteobacteria | FJHX01000023.1 |
| 387 | GCA_900062545.1 | 446 | 446 | Legionella pneumophila | 2532STDY5467591 | Proteobacteria | FJHZ01000023.1 |
| 388 | GCA_900062555.1 | 446 | 446 | Legionella pneumophila | 2532STDY5467578 | Proteobacteria | FJIB01000025.1 |
| 389 | GCA_900062565.1 | 446 | 446 | Legionella pneumophila | 2532STDY5467581 | Proteobacteria | FJIC01000024.1 |
| 390 | GCA_900062575.1 | 446 | 446 | Legionella pneumophila | 2532STDY5467577 | Proteobacteria | FJIE01000004.1 |
| 391 | GCA_900062585.1 | 446 | 446 | Legionella pneumophila | 2532STDY5467546 | Proteobacteria | FJIN01000024.1 |
| 392 | GCA_900062595.1 | 446 | 446 | Legionella pneumophila | 2532STDY5467565 | Proteobacteria | FJIO01000024.1 |
| 393 | GCA_900062605.1 | 446 | 446 | Legionella pneumophila | 2532STDY5467561 | Proteobacteria | FJIR01000021.1 |
| 394 | GCA_900062615.1 | 446 | 446 | Legionella pneumophila | 2532STDY5467559 | Proteobacteria | FJIQ01000021.1 |
| 395 | GCA_900062625.1 | 446 | 446 | Legionella pneumophila | 2532STDY5467628 | Proteobacteria | FJIU01000023.1 |
| 396 | GCA_900062635.1 | 446 | 446 | Legionella pneumophila | 2532STDY5467538 | Proteobacteria | FJIY01000023.1 |
| 397 | GCA_900062645.1 | 446 | 446 | Legionella pneumophila | 2532STDY5467545 | Proteobacteria | FJIX01000022.1 |
| 398 | GCA_900062655.1 | 446 | 446 | Legionella pneumophila | 2532STDY5467602 | Proteobacteria | FJJH01000024.1 |
| 399 | GCA_900062665.1 | 446 | 446 | Legionella pneumophila | 2532STDY5467562 | Proteobacteria | FJJI01000022.1 |
| 400 | GCA_900062675.1 | 446 | 446 | Legionella pneumophila | 2532STDY5467539 | Proteobacteria | FJJJ01000022.1 |
| 401 | GCA_900062685.1 | 446 | 446 | Legionella pneumophila | 2532STDY5467566 | Proteobacteria | FJJK01000024.1 |
| 402 | GCA_900062695.1 | 446 | 446 | Legionella pneumophila | 2532STDY5467572 | Proteobacteria | FJJM01000023.1 |
| 403 | GCA_900062705.1 | 446 | 446 | Legionella pneumophila | 2532STDY5467608 | Proteobacteria | FJJO01000023.1 |
| 404 | GCA_900062715.1 | 446 | 446 | Legionella pneumophila | 2532STDY5467564 | Proteobacteria | FJJN01000023.1 |
| 405 | GCA_900062725.1 | 446 | 446 | Legionella pneumophila | 2532STDY5467586 | Proteobacteria | FJJQ01000024.1 |
| 406 | GCA_900062735.1 | 446 | 446 | Legionella pneumophila | 2532STDY5467567 | Proteobacteria | FJJW01000021.1 |
| 407 | GCA_900062745.1 | 446 | 446 | Legionella pneumophila | 2532STDY5467598 | Proteobacteria | FJKA01000023.1 |
| 408 | GCA_900062755.1 | 446 | 446 | Legionella pneumophila | 2532STDY5467585 | Proteobacteria | FJKB01000023.1 |
| 409 | GCA_900063115.1 | 446 | 446 | Legionella pneumophila | 2532STDY5467558 | Proteobacteria | FJJS01000025.1 |
| 410 | GCA_900063125.1 | 446 | 446 | Legionella pneumophila | 2532STDY5467615 | Proteobacteria | FJKW01000024.1 |
| 411 | GCA_900063135.1 | 446 | 446 | Legionella pneumophila | 2532STDY5467587 | Proteobacteria | FJKZ01000024.1 |
| 412 | GCA_900063145.1 | 446 | 446 | Legionella pneumophila | 2532STDY5467612 | Proteobacteria | FJKX01000024.1 |
| 413 | GCA_900063835.1 | 446 | 446 | Legionella pneumophila | 2532STDY5467552 | Proteobacteria | FJJE01000022.1 |
| 414 | GCA_900063845.1 | 446 | 446 | Legionella pneumophila | 2532STDY5467605 | Proteobacteria | FJKD01000023.1 |
| 415 | GCA_900063855.1 | 446 | 446 | Legionella pneumophila | 2532STDY5467597 | Proteobacteria | FJKG01000023.1 |
| 416 | GCA_900063865.1 | 446 | 446 | Legionella pneumophila | 2532STDY5467594 | Proteobacteria | FJKH01000022.1 |
| 417 | GCA_900063875.1 | 446 | 446 | Legionella pneumophila | 2532STDY5467589 | Proteobacteria | FJKN01000025.1 |
| 418 | GCA_900063885.1 | 446 | 446 | Legionella pneumophila | 2532STDY5467601 | Proteobacteria | FJKO01000022.1 |
| 419 | GCA_900063895.1 | 446 | 446 | Legionella pneumophila | 2532STDY5467620 | Proteobacteria | FJLE01000025.1 |
| 420 | GCA_900064205.1 | 446 | 446 | Legionella pneumophila | 2532STDY5467618 | Proteobacteria | FJLI01000003.1 |
| 421 | GCA_900064315.1 | 446 | 446 | Legionella pneumophila | 2532STDY5467599 | Proteobacteria | FJKJ01000024.1 |
| 422 | GCA_900064325.1 | 446 | 446 | Legionella pneumophila | 2532STDY5467606 | Proteobacteria | FJKL01000024.1 |
| 423 | GCA_900064495.1 | 446 | 446 | Legionella pneumophila | 2532STDY5467534 | Proteobacteria | FJIW01000023.1 |
| 424 | GCA_900064755.1 | 446 | 446 | Legionella pneumophila | 2532STDY5467579 | Proteobacteria | FJIV01000020.1 |
| 425 | GCA_900064765.1 | 446 | 446 | Legionella pneumophila | 2532STDY5467590 | Proteobacteria | FJKI01000024.1 |
| 426 | GCA_900073005.1 | 446 | 446 | Legionella pneumophila | LP_617 | Proteobacteria | FJOC01000028.1 |
| 427 | GCA_001467765.1 | 456 | 456 | Legionella jordanis | BL-540 | Proteobacteria | LNYJ01000003.1 |
| 428 | GCA_000353775.1 | 1094483 | 470 | Acinetobacter baumannii ABNIH19 | ABNIH19 | Proteobacteria | APBH01000063.1 |
| 429 | GCA_000353955.1 | 1094470 | 470 | Acinetobacter baumannii ABNIH6 | ABNIH6 | Proteobacteria | APAX01000087.1 |
| 430 | GCA_000353975.1 | 1094471 | 470 | Acinetobacter baumannii ABNIH7 | ABNIH7 | Proteobacteria | APAY01000451.1 |
| 431 | GCA_000353995.1 | 1094474 | 470 | Acinetobacter baumannii ABNIH10 | ABNIH10 | Proteobacteria | APAZ01000253.1 |
| 432 | GCA_000354015.1 | 1094475 | 470 | Acinetobacter baumannii ABNIH11 | ABNIH11 | Proteobacteria | APBA01000112.1 |
| 433 | GCA_000580595.1 | 1310619 | 470 | Acinetobacter baumannii 1419130 | 1419130 | Proteobacteria | JEWL01000035.1 |
| 434 | GCA_000580635.1 | 1310647 | 470 | Acinetobacter baumannii 1461402 | 1461402 | Proteobacteria | JEWP01000001.1 |
| 435 | GCA_000580875.1 | 1310613 | 470 | Acinetobacter baumannii 1295743 | 1295743 | Proteobacteria | JEWH01000023.1 |
| 436 | GCA_000580995.1 | 1310611 | 470 | Acinetobacter baumannii 466760 | 466760 | Proteobacteria | JEXB01000001.1 |
| 437 | GCA_000581195.1 | 1310651 | 470 | Acinetobacter baumannii 230853 | 230853 | Proteobacteria | JEWW01000002.1 |
| 438 | GCA_000582235.1 | 1310680 | 470 | Acinetobacter baumannii 1239920 | 1239920 | Proteobacteria | JEXZ01000002.1 |
| 439 | GCA_000582255.1 | 1310683 | 470 | Acinetobacter baumannii 1566109 | 1566109 | Proteobacteria | JEYB01000087.1 |
| 440 | GCA_000682355.1 | 1310685 | 470 | Acinetobacter baumannii 72431 | 72431 | Proteobacteria | JMNZ01000050.1 |
| 441 | GCA_000805365.1 | 470 | 470 | Acinetobacter baumannii | 1 | Proteobacteria | JSAO01000006.1 |
| 442 | GCA_001028315.1 | 1409926 | 470 | Acinetobacter baumannii MRSN 58 | MRSN 58 | Proteobacteria | JPHW01000032.1 |
| 443 | GCA_001432425.1 | 470 | 470 | Acinetobacter baumannii | ABBL014 | Proteobacteria | LLCU01000093.1 |
| 444 | GCA_001433005.1 | 470 | 470 | Acinetobacter baumannii | ABBL038 | Proteobacteria | LLDS01000053.1 |
| 445 | GCA_001578145.1 | 470 | 470 | Acinetobacter baumannii | XH858 | Proteobacteria | CP014528.1 |
| 446 | GCA_000690355.2 | 1458258 | 518 | Bordetella bronchiseptica B18-5 (C3) | B18-5 (C3) | Proteobacteria | JGWP01000092.1 |
| 447 | GCA_000690635.1 | 1331233 | 518 | Bordetella bronchiseptica MBORD762 | MBORD762 | Proteobacteria | JHBR01000046.1 |
| 448 | GCA_000024245.1 | 622759 | 542 | Zymomonas mobilis subsp. mobilis NCIMB 11163 | NCIMB 11163 | Proteobacteria | CP001723.1 |
| 449 | GCA_000218875.1 | 579138 | 542 | Zymomonas mobilis subsp. pomaceae ATCC 29192 | ATCC 29192 | Proteobacteria | CP002866.1 |
| 450 | GCA_001472275.1 | 548 | 548 | Enterobacter aerogenes | SMART_429 | Proteobacteria | LPQB01000006.1 |
| 451 | GCA_001518035.1 | 548 | 548 | Enterobacter aerogenes | GN02710 | Proteobacteria | LRCB01000137.1 |
| 452 | GCA_000743785.2 | 549 | 549 | Pantoea agglomerans | 4 | Proteobacteria | JPOT02000001.1 |
| 453 | GCA_000286275.1 | 1211025 | 550 | Enterobacter cloacae subsp. cloacae ENHKU01 | ENHKU01 | Proteobacteria | CP003737.1 |
| 454 | GCA_000496775.1 | 1399146 | 550 | Enterobacter cloacae S611 | S611 | Proteobacteria | AXOM01000016.1 |
| 455 | GCA_000534275.1 | 1400147 | 550 | Enterobacter cloacae UCI 24 | UCI 24 | Proteobacteria | KI973153.1 |
| 456 | GCA_000692355.1 | 1400148 | 550 | Enterobacter cloacae UCI 29 | UCI 29 | Proteobacteria | KK736271.1 |
| 457 | GCA_000784865.1 | 550 | 550 | Enterobacter cloacae | ECNIH4 | Proteobacteria | CP009850.1 |
| 458 | GCA_000965825.1 | 550 | 550 | Enterobacter cloacae | CIDEIMsCOL5 | Proteobacteria | JZKP01000063.1 |
| 459 | GCA_000966005.1 | 550 | 550 | Enterobacter cloacae | CIDEIMsCOL4 | Proteobacteria | JZKO01000065.1 |
| 460 | GCA_001317225.1 | 336306 | 550 | Enterobacter cloacae subsp. cloacae | ST434:953099839 | Proteobacteria | LIEW01000028.1 |
| 461 | GCA_001441705.1 | 550 | 550 | Enterobacter cloacae | 8706 | Proteobacteria | LLXN01000108.1 |
| 462 | GCA_001518525.1 | 550 | 550 | Enterobacter cloacae | GN02618 | Proteobacteria | LRBY01000003.1 |
| 463 | GCA_000270125.2 | 932677 | 553 | Pantoea ananatis AJ13355 | AJ13355 | Proteobacteria | AP012032.2 |
| 464 | GCA_000283875.1 | 1123863 | 553 | Pantoea ananatis LMG 5342 | LMG 5342 | Proteobacteria | HE617160.1 |
| 465 | GCA_000294535.1 | 1218933 | 554 | Pectobacterium carotovorum subsp. carotovorum PCC21 | PCC21 | Proteobacteria | CP003776.1 |
| 466 | GCA_000808145.1 | 180957 | 554 | Pectobacterium carotovorum subsp. brasiliense | YC D62 | Proteobacteria | JUJD01000008.1 |
| 467 | GCA_000808295.1 | 180957 | 554 | Pectobacterium carotovorum subsp. brasiliense | YC T31 | Proteobacteria | JUJL01000008.1 |
| 468 | GCA_000808315.1 | 180957 | 554 | Pectobacterium carotovorum subsp. brasiliense | YC T3 | Proteobacteria | JUJM01000011.1 |
| 469 | GCA_000017745.1 | 331111 | 562 | Escherichia coli O139:H28 str. E24377A | E24377A | Proteobacteria | CP000800.1 |
| 470 | GCA_000267025.1 | 1005535 | 562 | Escherichia coli EC1738 | EC1738 | Proteobacteria | AKMN01000044.1 |
| 471 | GCA_000267865.2 | 1005494 | 562 | Escherichia coli PA10 | PA10 | Proteobacteria | JH708741.1 |
| 472 | GCA_000304875.2 | 1005452 | 562 | Escherichia coli 5.2239 | 5.2239 | Proteobacteria | JH970984.1 |
| 473 | GCA_000316885.2 | 1005408 | 562 | Escherichia coli 95.0943 | 95.0943 | Proteobacteria | KB000904.1 |
| 474 | GCA_000335135.1 | 1005439 | 562 | Escherichia coli 99.1781 | 99.1781 | Proteobacteria | AOEQ01000009.1 |
| 475 | GCA_000335295.2 | 1005440 | 562 | Escherichia coli 99.1753 | 99.1753 | Proteobacteria | AOED01000081.1 |
| 476 | GCA_000335315.2 | 1005438 | 562 | Escherichia coli 99.1775 | 99.1775 | Proteobacteria | AOEE01000071.1 |
| 477 | GCA_000340455.2 | 1081887 | 562 | Escherichia coli 09BKT078844 | 09BKT078844 | Proteobacteria | KB452795.1 |
| 478 | GCA_000351585.1 | 1182667 | 562 | Escherichia coli KTE60 | KTE60 | Proteobacteria | KB732732.1 |
| 479 | GCA_000352105.1 | 1169329 | 562 | Escherichia coli KTE9 | KTE9 | Proteobacteria | KB732995.1 |
| 480 | GCA_000357165.1 | 1116056 | 562 | Escherichia coli P0299917.10 | P0299917.10 | Proteobacteria | APYK01000102.1 |
| 481 | GCA_000357185.2 | 1116048 | 562 | Escherichia coli P0299917.2 | P0299917.2 | Proteobacteria | APYL01000006.1 |
| 482 | GCA_000357205.2 | 1116049 | 562 | Escherichia coli P0299917.3 | P0299917.3 | Proteobacteria | APYM01000020.1 |
| 483 | GCA_000357305.2 | 1116050 | 562 | Escherichia coli P0299917.4 | P0299917.4 | Proteobacteria | APYN01000006.1 |
| 484 | GCA_000357325.1 | 1116051 | 562 | Escherichia coli P0299917.5 | P0299917.5 | Proteobacteria | APYO01000007.1 |
| 485 | GCA_000357345.2 | 1116052 | 562 | Escherichia coli P0299917.6 | P0299917.6 | Proteobacteria | APYP01000006.1 |
| 486 | GCA_000357365.2 | 1116053 | 562 | Escherichia coli P0299917.7 | P0299917.7 | Proteobacteria | APYQ01000007.1 |
| 487 | GCA_000357505.2 | 1116055 | 562 | Escherichia coli P0299917.9 | P0299917.9 | Proteobacteria | APYS01000112.1 |
| 488 | GCA_000357685.2 | 1125651 | 562 | Escherichia coli P0304799.3 | P0304799.3 | Proteobacteria | APZX01000274.1 |
| 489 | GCA_000358795.2 | 1116054 | 562 | Escherichia coli P0299917.8 | P0299917.8 | Proteobacteria | APYR01000109.1 |
| 490 | GCA_000398885.1 | 1169351 | 562 | Escherichia coli KTE33 | KTE33 | Proteobacteria | KB976087.1 |
| 491 | GCA_000459555.1 | 1281153 | 562 | Escherichia coli KOEGE 3 (4a) | KOEGE 3 (4a) | Proteobacteria | KE701179.1 |
| 492 | GCA_000459855.1 | 1281168 | 562 | Escherichia coli KOEGE 71 (186a) | KOEGE 71 (186a) | Proteobacteria | KE701379.1 |
| 493 | GCA_000459915.1 | 1281171 | 562 | Escherichia coli KOEGE 118 (317a) | KOEGE 118 (317a) | Proteobacteria | KE701414.1 |
| 494 | GCA_000460355.1 | 1281194 | 562 | Escherichia coli UMEA 3151-1 | UMEA 3151-1 | Proteobacteria | KE701683.1 |
| 495 | GCA_000461795.1 | 1281275 | 562 | Escherichia coli UMEA 3899-1 | UMEA 3899-1 | Proteobacteria | KE702684.1 |
| 496 | GCA_000461915.2 | 1151226 | 562 | Escherichia coli B26-2 | B26-2 | Proteobacteria | AVQV01000011.1 |
| 497 | GCA_000461955.2 | 1151213 | 562 | Escherichia coli B102 | B102 | Proteobacteria | AVQS01000034.1 |
| 498 | GCA_000461995.2 | 1151214 | 562 | Escherichia coli B107 | B107 | Proteobacteria | AVQT01000036.1 |
| 499 | GCA_000462025.2 | 1151225 | 562 | Escherichia coli B26-1 | B26-1 | Proteobacteria | AVQU01000037.1 |
| 500 | GCA_000462485.2 | 1151234 | 562 | Escherichia coli B103 | B103 | Proteobacteria | AVRS01000033.1 |
| 501 | GCA_000462505.2 | 1151243 | 562 | Escherichia coli B104 | B104 | Proteobacteria | AVRT01000033.1 |
| 502 | GCA_000462525.2 | 1151247 | 562 | Escherichia coli B105 | B105 | Proteobacteria | AVRU01000004.1 |
| 503 | GCA_000462545.2 | 1151248 | 562 | Escherichia coli B106 | B106 | Proteobacteria | AVRV01000031.1 |
| 504 | GCA_000462685.2 | 1151227 | 562 | Escherichia coli B40-1 | B40-1 | Proteobacteria | AVSD01000007.1 |
| 505 | GCA_000462705.2 | 1151228 | 562 | Escherichia coli B40-2 | B40-2 | Proteobacteria | AVSE01000035.1 |
| 506 | GCA_000462725.2 | 1151246 | 562 | Escherichia coli B49-2 | B49-2 | Proteobacteria | AVSF01000004.1 |
| 507 | GCA_000462765.2 | 1151239 | 562 | Escherichia coli B83 | B83 | Proteobacteria | AVSH01000033.1 |
| 508 | GCA_000462785.2 | 1151240 | 562 | Escherichia coli B84 | B84 | Proteobacteria | AVSI01000002.1 |
| 509 | GCA_000462805.2 | 1151241 | 562 | Escherichia coli B85 | B85 | Proteobacteria | AVSJ01000006.1 |
| 510 | GCA_000462825.2 | 1151242 | 562 | Escherichia coli B86 | B86 | Proteobacteria | AVSK01000012.1 |
| 511 | GCA_000599665.1 | 562 | 562 | Escherichia coli | ST2747 | Proteobacteria | CP007392.1 |
| 512 | GCA_000599685.1 | 562 | 562 | Escherichia coli | ST2747 | Proteobacteria | CP007393.1 |
| 513 | GCA_000599705.1 | 562 | 562 | Escherichia coli | ST2747 | Proteobacteria | CP007394.1 |
| 514 | GCA_000601195.1 | 1444161 | 562 | Escherichia coli 1-176-05_S3_C2 | 1-176-05_S3_C2 | Proteobacteria | JHDF01000008.1 |
| 515 | GCA_000614255.1 | 1446644 | 562 | Escherichia coli O157:H7 str. 2011EL-2114 | 2011EL-2114 | Proteobacteria | JHKH01000012.1 |
| 516 | GCA_000614645.2 | 1446643 | 562 | Escherichia coli O157:H7 str. 2011EL-2113 | 2011EL-2113 | Proteobacteria | JHKI01000034.1 |
| 517 | GCA_000614805.2 | 1446656 | 562 | Escherichia coli O157:H7 str. F7350 | F7350 | Proteobacteria | JHJN01000063.1 |
| 518 | GCA_000615745.2 | 1446634 | 562 | Escherichia coli O157:H7 str. 2011EL-2103 | 2011EL-2103 | Proteobacteria | JHKR01000093.1 |
| 519 | GCA_000615785.2 | 1446635 | 562 | Escherichia coli O157:H7 str. 2011EL-2104 | 2011EL-2104 | Proteobacteria | JHKQ01000088.1 |
| 520 | GCA_000615815.2 | 1446636 | 562 | Escherichia coli O157:H7 str. 2011EL-2105 | 2011EL-2105 | Proteobacteria | JHKP01000065.1 |
| 521 | GCA_000615865.2 | 1446637 | 562 | Escherichia coli O157:H7 str. 2011EL-2106 | 2011EL-2106 | Proteobacteria | JHKO01000118.1 |
| 522 | GCA_000615925.2 | 1446638 | 562 | Escherichia coli O157:H7 str. 2011EL-2107 | 2011EL-2107 | Proteobacteria | JHKN01000091.1 |
| 523 | GCA_000615965.2 | 1446639 | 562 | Escherichia coli O157:H7 str. 2011EL-2108 | 2011EL-2108 | Proteobacteria | JHKM01000041.1 |
| 524 | GCA_000616005.2 | 1446640 | 562 | Escherichia coli O157:H7 str. 2011EL-2109 | 2011EL-2109 | Proteobacteria | JHKL01000081.1 |
| 525 | GCA_000616035.2 | 1446641 | 562 | Escherichia coli O157:H7 str. 2011EL-2111 | 2011EL-2111 | Proteobacteria | JHKK01000026.1 |
| 526 | GCA_000616075.2 | 1446642 | 562 | Escherichia coli O157:H7 str. 2011EL-2112 | 2011EL-2112 | Proteobacteria | JHKJ01000066.1 |
| 527 | GCA_000617465.2 | 1446524 | 562 | Escherichia coli O111:NM str. 2010C-4735 | 2010C-4735 | Proteobacteria | JHMU01000073.1 |
| 528 | GCA_000618225.2 | 1446657 | 562 | Escherichia coli O157:H7 str. F7377 | F7377 | Proteobacteria | JHJM01000101.1 |
| 529 | GCA_000619325.2 | 1446580 | 562 | Escherichia coli O123:H11 str. 2009C-3307 | 2009C-3307 | Proteobacteria | JHHB01000001.1 |
| 530 | GCA_000704985.1 | 1444084 | 562 | Escherichia coli 3-105-05_S1_C2 | 3-105-05_S1_C2 | Proteobacteria | JNSA01000028.1 |
| 531 | GCA_000797815.1 | 562 | 562 | Escherichia coli | CVM N41556PS | Proteobacteria | JUBQ01000063.1 |
| 532 | GCA_000797915.1 | 562 | 562 | Escherichia coli | CVM N41498PS | Proteobacteria | JUBR01000061.1 |
| 533 | GCA_001281885.1 | 562 | 562 | Escherichia coli | STEC 2257 | Proteobacteria | LGBF01000030.1 |
| 534 | GCA_001281915.1 | 562 | 562 | Escherichia coli | STEC 2821 | Proteobacteria | LGBI01000040.1 |
| 535 | GCA_001282065.1 | 562 | 562 | Escherichia coli | STEC 2820 | Proteobacteria | LGBQ01000093.1 |
| 536 | GCA_001284745.1 | 562 | 562 | Escherichia coli | 401709_aEPEC | Proteobacteria | CYCR01000027.1 |
| 537 | GCA_001285705.1 | 562 | 562 | Escherichia coli | 402767 | Proteobacteria | CYET01000020.1 |
| 538 | GCA_001297965.1 | 562 | 562 | Escherichia coli | STEC 915 | Proteobacteria | LFUH01000095.1 |
| 539 | GCA_001297985.1 | 562 | 562 | Escherichia coli | STEC 623 | Proteobacteria | LFUB01000081.1 |
| 540 | GCA_001309565.1 | 562 | 562 | Escherichia coli | PARC448 | Proteobacteria | LDYO01000075.1 |
| 541 | GCA_001309965.1 | 562 | 562 | Escherichia coli | CO283 | Proteobacteria | LEAI01000125.1 |
| 542 | GCA_001419785.1 | 562 | 562 | Escherichia coli | 33 | Proteobacteria | LIUW01000013.1 |
| 543 | GCA_001455035.1 | 562 | 562 | Escherichia coli | 22593 | Proteobacteria | LMBK01000190.1 |
| 544 | GCA_001519145.1 | 562 | 562 | Escherichia coli | GN02606 | Proteobacteria | LQUQ01000101.1 |
| 545 | GCA_001521225.1 | 562 | 562 | Escherichia coli | GN02350 | Proteobacteria | LQTE01000121.1 |
| 546 | GCA_001572045.1 | 562 | 562 | Escherichia coli | CFSAN025093 | Proteobacteria | LPXY01000033.1 |
| 547 | GCA_001572205.1 | 562 | 562 | Escherichia coli | CFSAN025114 | Proteobacteria | LPYO01000230.1 |
| 548 | GCA_001572345.1 | 562 | 562 | Escherichia coli | CFSAN025116 | Proteobacteria | LPYQ01000200.1 |
| 549 | GCA_001572445.1 | 562 | 562 | Escherichia coli | CFSAN025127 | Proteobacteria | LPYZ01000229.1 |
| 550 | GCA_001575465.1 | 562 | 562 | Escherichia coli | G222 | Proteobacteria | LOOL01000028.1 |
| 551 | GCA_001575625.1 | 562 | 562 | Escherichia coli | G236 | Proteobacteria | LOOR01000015.1 |
| 552 | GCA_001576035.1 | 562 | 562 | Escherichia coli | G27 | Proteobacteria | LOOY01000001.1 |
| 553 | GCA_001576095.1 | 562 | 562 | Escherichia coli | G303 | Proteobacteria | LOPE01000051.1 |
| 554 | GCA_001576205.1 | 562 | 562 | Escherichia coli | G301 | Proteobacteria | LOPD01000001.1 |
| 555 | GCA_001576325.1 | 562 | 562 | Escherichia coli | G28 | Proteobacteria | LOOZ01000025.1 |
| 556 | GCA_001576455.1 | 562 | 562 | Escherichia coli | G51 | Proteobacteria | LOPO01000094.1 |
| 557 | GCA_000191665.1 | 981367 | 564 | Escherichia fergusonii ECD227 | ECD227 | Proteobacteria | CM001142.1 |
| 558 | GCA_000248015.2 | 1115512 | 565 | Escherichia hermannii NBRC 105704 | NBRC 105704 | Proteobacteria | BAFF01000030.1 |
| 559 | GCA_000239255.1 | 1002364 | 569 | Hafnia alvei ATCC 51873 | ATCC 51873 | Proteobacteria | JH417508.1 |
| 560 | GCA_000300675.1 | 1203545 | 573 | Klebsiella pneumoniae subsp. pneumoniae WGLW2 | WGLW2 | Proteobacteria | JH930419.1 |
| 561 | GCA_000300675.1 | 1203545 | 573 | Klebsiella pneumoniae subsp. pneumoniae WGLW2 | WGLW2 | Proteobacteria | JH930421.1 |
| 562 | GCA_000316265.2 | 1236101 | 573 | Klebsiella pneumoniae JHCK1 | JHCK1 | Proteobacteria | ANGH02000148.1 |
| 563 | GCA_000316265.2 | 1236101 | 573 | Klebsiella pneumoniae JHCK1 | JHCK1 | Proteobacteria | ANGH02000193.1 |
| 564 | GCA_000409125.1 | 1284817 | 573 | Klebsiella pneumoniae UHKPC57 | UHKPC57 | Proteobacteria | ARPR01000165.1 |
| 565 | GCA_000417465.1 | 1284818 | 573 | Klebsiella pneumoniae UHKPC179 | UHKPC179 | Proteobacteria | ARSM01000009.1 |
| 566 | GCA_000474635.1 | 1381121 | 573 | Klebsiella pneumoniae 303K | 303K | Proteobacteria | AVAN01000015.1 |
| 567 | GCA_000492775.1 | 1328378 | 573 | Klebsiella pneumoniae MGH 46 | MGH 46 | Proteobacteria | KI535585.1 |
| 568 | GCA_000529945.1 | 1432553 | 573 | Klebsiella pneumoniae IS46 | IS46 | Proteobacteria | CBWL010000661.1 |
| 569 | GCA_000567405.1 | 1328405 | 573 | Klebsiella pneumoniae BIDMC 11 | BIDMC 11 | Proteobacteria | KK036861.1 |
| 570 | GCA_000567405.1 | 1328405 | 573 | Klebsiella pneumoniae BIDMC 11 | BIDMC 11 | Proteobacteria | KK036856.1 |
| 571 | GCA_000688155.1 | 1438786 | 573 | Klebsiella pneumoniae MGH 63 | MGH 63 | Proteobacteria | JJNB01000019.1 |
| 572 | GCA_000688215.1 | 1438798 | 573 | Klebsiella pneumoniae MGH 75 | MGH 75 | Proteobacteria | JJNK01000020.1 |
| 573 | GCA_000694235.1 | 1438772 | 573 | Klebsiella pneumoniae CHS 66 | CHS 66 | Proteobacteria | KK737377.1 |
| 574 | GCA_000694535.1 | 1438788 | 573 | Klebsiella pneumoniae MGH 65 | MGH 65 | Proteobacteria | KK737534.1 |
| 575 | GCA_000707985.1 | 1329843 | 573 | Klebsiella pneumoniae BIDMC 33B | BIDMC 33B | Proteobacteria | KL407346.1 |
| 576 | GCA_000733255.1 | 1406314 | 573 | Klebsiella pneumoniae subsp. pneumoniae PittNDM01 | PittNDM01 | Proteobacteria | CP006798.1 |
| 577 | GCA_000764615.1 | 573 | 573 | Klebsiella pneumoniae | PMK1 | Proteobacteria | CP008929.1 |
| 578 | GCA_000764635.1 | 573 | 573 | Klebsiella pneumoniae | BAMC 07-18 | Proteobacteria | JRQE01000001.1 |
| 579 | GCA_000788005.1 | 573 | 573 | Klebsiella pneumoniae | 101712 | Proteobacteria | JSZI01000009.1 |
| 580 | GCA_000943095.1 | 573 | 573 | Klebsiella pneumoniae | ST15 NDM-1 | Proteobacteria | CDQG01000025.1 |
| 581 | GCA_001031325.1 | 573 | 573 | Klebsiella pneumoniae | CHS112 | Proteobacteria | KQ088280.1 |
| 582 | GCA_001032775.1 | 573 | 573 | Klebsiella pneumoniae | CHS201 | Proteobacteria | KQ088952.1 |
| 583 | GCA_001033455.1 | 573 | 573 | Klebsiella pneumoniae | MGH81 | Proteobacteria | KQ089253.1 |
| 584 | GCA_001033725.1 | 573 | 573 | Klebsiella pneumoniae | MGH101 | Proteobacteria | KQ089465.1 |
| 585 | GCA_001033755.1 | 573 | 573 | Klebsiella pneumoniae | MGH111 | Proteobacteria | KQ089480.1 |
| 586 | GCA_001033835.1 | 573 | 573 | Klebsiella pneumoniae | MGH115 | Proteobacteria | KQ089541.1 |
| 587 | GCA_001033885.1 | 573 | 573 | Klebsiella pneumoniae | MGH117 | Proteobacteria | KQ089551.1 |
| 588 | GCA_001033925.1 | 573 | 573 | Klebsiella pneumoniae | MGH124 | Proteobacteria | KQ089570.1 |
| 589 | GCA_001033925.1 | 573 | 573 | Klebsiella pneumoniae | MGH124 | Proteobacteria | KQ089573.1 |
| 590 | GCA_001034065.1 | 573 | 573 | Klebsiella pneumoniae | BIDMC90 | Proteobacteria | KQ089642.1 |
| 591 | GCA_001034365.1 | 573 | 573 | Klebsiella pneumoniae | UCI96 | Proteobacteria | KQ089764.1 |
| 592 | GCA_001185865.1 | 573 | 573 | Klebsiella pneumoniae | CHS175 | Proteobacteria | KQ236853.1 |
| 593 | GCA_001307175.1 | 573 | 573 | Klebsiella pneumoniae | KP617 | Proteobacteria | CP012753.1 |
| 594 | GCA_001316865.1 | 72407 | 573 | Klebsiella pneumoniae subsp. pneumoniae | ST14:944535499 | Proteobacteria | LIDW01000003.1 |
| 595 | GCA_001462705.1 | 573 | 573 | Klebsiella pneumoniae | K57-33 | Proteobacteria | LNGX01000156.1 |
| 596 | GCA_001463165.1 | 573 | 573 | Klebsiella pneumoniae | U25 | Proteobacteria | CP012043.1 |
| 597 | GCA_001463475.1 | 573 | 573 | Klebsiella pneumoniae | 50884375 | Proteobacteria | LNIS01000122.1 |
| 598 | GCA_001521895.1 | 72407 | 573 | Klebsiella pneumoniae subsp. pneumoniae | NUHL24835 | Proteobacteria | CP014004.1 |
| 599 | GCA_001574505.1 | 573 | 573 | Klebsiella pneumoniae | U41 | Proteobacteria | LSTN01000108.1 |
| 600 | GCA_001596905.1 | 573 | 573 | Klebsiella pneumoniae | KPOII-4 | Proteobacteria | JXIW01000052.1 |
| 601 | GCA_001596915.1 | 573 | 573 | Klebsiella pneumoniae | KPOII-2 | Proteobacteria | JXIV01000036.1 |
| 602 | GCA_001596925.1 | 573 | 573 | Klebsiella pneumoniae | KPOII-2 | Proteobacteria | JUBO01000066.1 |
| 603 | GCA_001596935.1 | 573 | 573 | Klebsiella pneumoniae | KPOII-1 | Proteobacteria | JUBN01000046.1 |
| 604 | GCA_001596985.1 | 573 | 573 | Klebsiella pneumoniae | KPOII-7 | Proteobacteria | JXIY01000037.1 |
| 605 | GCA_001596995.1 | 573 | 573 | Klebsiella pneumoniae | KPOII-10 | Proteobacteria | JXJB01000058.1 |
| 606 | GCA_001597015.1 | 573 | 573 | Klebsiella pneumoniae | KP-33P | Proteobacteria | JXJG01000008.1 |
| 607 | GCA_001597095.1 | 573 | 573 | Klebsiella pneumoniae | KPOII-1 | Proteobacteria | JXIU01000015.1 |
| 608 | GCA_001597115.1 | 573 | 573 | Klebsiella pneumoniae | KPOII-6 | Proteobacteria | JXIX01000053.1 |
| 609 | GCA_001597145.1 | 573 | 573 | Klebsiella pneumoniae | KPOII-8 | Proteobacteria | JXIZ01000056.1 |
| 610 | GCA_001597165.1 | 573 | 573 | Klebsiella pneumoniae | KPOII-9 | Proteobacteria | JXJA01000044.1 |
| 611 | GCA_001597175.1 | 573 | 573 | Klebsiella pneumoniae | KPEII-1 | Proteobacteria | JXJC01000056.1 |
| 612 | GCA_001597195.1 | 573 | 573 | Klebsiella pneumoniae | KPEII-2 | Proteobacteria | JXJD01000059.1 |
| 613 | GCA_001597225.1 | 573 | 573 | Klebsiella pneumoniae | KPEII-3 | Proteobacteria | JXJE01000058.1 |
| 614 | GCA_001597245.1 | 573 | 573 | Klebsiella pneumoniae | KP-10 | Proteobacteria | LDOY01000067.1 |
| 615 | GCA_001219605.1 | 630 | 630 | Yersinia enterocolitica | YE04/02 | Proteobacteria | CTKX01000003.1 |
| 616 | GCA_001319885.1 | 633 | 633 | Yersinia pseudotuberculosis | OK6088 | Proteobacteria | CGGM01000007.1 |
| 617 | GCA_001131685.1 | 634 | 634 | Yersinia bercovieri | 127/84 | Proteobacteria | CQBU01000002.1 |
| 618 | GCA_000612075.2 | 1448139 | 644 | Aeromonas hydrophila YL17 | YL17 | Proteobacteria | CP007518.2 |
| 619 | GCA_001270765.1 | 648 | 648 | Aeromonas caviae | 429865 | Proteobacteria | LIIX01000004.1 |
| 620 | GCA_001446895.1 | 656 | 656 | Aeromonas allosaccharophila | TTU2014-159ASC | Proteobacteria | LKKI01000013.1 |
| 621 | GCA_000006745.1 | 243277 | 666 | Vibrio cholerae O1 biovar El Tor str. N16961 | N16961 | Proteobacteria | AE003852.1 |
| 622 | GCA_000016245.1 | 345073 | 666 | Vibrio cholerae O395 | O395 | Proteobacteria | CP000627.1 |
| 623 | GCA_000021605.1 | 579112 | 666 | Vibrio cholerae M66-2 | M66-2 | Proteobacteria | CP001233.1 |
| 624 | GCA_000021625.1 | 345073 | 666 | Vibrio cholerae O395 | O395 | Proteobacteria | CP001235.1 |
| 625 | GCA_000022585.1 | 593588 | 666 | Vibrio cholerae MJ-1236 | MJ-1236 | Proteobacteria | CP001485.1 |
| 626 | GCA_000153865.1 | 412967 | 666 | Vibrio cholerae MAK 757 | MAK 757 | Proteobacteria | GG774578.1 |
| 627 | GCA_000153945.1 | 417399 | 666 | Vibrio cholerae NCTC 8457 | NCTC 8457 | Proteobacteria | DS178000.1 |
| 628 | GCA_000153965.1 | 417400 | 666 | Vibrio cholerae B33 | B33 | Proteobacteria | DS178064.1 |
| 629 | GCA_000166455.2 | 914149 | 666 | Vibrio cholerae O1 str. 2010EL-1786 | 2010EL-1786 | Proteobacteria | CP003069.1 |
| 630 | GCA_000167935.2 | 345076 | 666 | Vibrio cholerae V52 | V52 | Proteobacteria | KQ410498.1 |
| 631 | GCA_000168915.3 | 412614 | 666 | Vibrio cholerae 2740-80 | 2740-80 | Proteobacteria | KQ257329.1 |
| 632 | GCA_000174275.1 | 593589 | 666 | Vibrio cholerae RC9 | RC9 | Proteobacteria | ACHX01000011.1 |
| 633 | GCA_000174315.1 | 417400 | 666 | Vibrio cholerae B33 | B33 | Proteobacteria | ACHZ01000017.1 |
| 634 | GCA_000174335.1 | 593587 | 666 | Vibrio cholerae BX 330286 | BX 330286 | Proteobacteria | ACIA01000006.1 |
| 635 | GCA_000175695.1 | 661513 | 666 | Vibrio cholerae CIRS101 | CIRS 101 | Proteobacteria | ACVW01000011.1 |
| 636 | GCA_000176395.1 | 675807 | 666 | Vibrio cholerae RC27 | RC27 | Proteobacteria | ADAI01000041.1 |
| 637 | GCA_000176435.1 | 675808 | 666 | Vibrio cholerae INDRE 91/1 | INDRE 91/1 | Proteobacteria | ADAK01000056.1 |
| 638 | GCA_000220725.2 | 991935 | 666 | Vibrio cholerae HC-49A2 | HC-49A2 | Proteobacteria | AFOM01000018.1 |
| 639 | GCA_000220745.3 | 991930 | 666 | Vibrio cholerae HCUF01 | HCUF01 | Proteobacteria | AFOO01000018.1 |
| 640 | GCA_000221345.1 | 991961 | 666 | Vibrio cholerae HC-40A1 | HC-40A1 | Proteobacteria | GL988221.1 |
| 641 | GCA_000221365.1 | 991965 | 666 | Vibrio cholerae HC-48A1 | HC-48A1 | Proteobacteria | GL988437.1 |
| 642 | GCA_000221385.1 | 991984 | 666 | Vibrio cholerae HC-70A1 | HC-70A1 | Proteobacteria | GL988603.1 |
| 643 | GCA_000221425.1 | 991989 | 666 | Vibrio cholerae HFU-02 | HFU-02 | Proteobacteria | GL988896.1 |
| 644 | GCA_000221485.1 | 991959 | 666 | Vibrio cholerae HC-38A1 | HC-38A1 | Proteobacteria | GL989435.1 |
| 645 | GCA_000234375.2 | 991950 | 666 | Vibrio cholerae HC-06A1 | HC-06A1 | Proteobacteria | AGUI01000118.1 |
| 646 | GCA_000234395.2 | 991933 | 666 | Vibrio cholerae HC-23A1 | HC-23A1 | Proteobacteria | AGUM01000011.1 |
| 647 | GCA_000234415.2 | 991955 | 666 | Vibrio cholerae HC-28A1 | HC-28A1 | Proteobacteria | AGUN01000124.1 |
| 648 | GCA_000234435.2 | 991938 | 666 | Vibrio cholerae HC-43A1 | HC-43A1 | Proteobacteria | AGUQ01000018.1 |
| 649 | GCA_000234455.3 | 991942 | 666 | Vibrio cholerae HC-61A1 | HC-61A1 | Proteobacteria | AGUS01000010.1 |
| 650 | GCA_000234865.1 | 991966 | 666 | Vibrio cholerae HC-48B2 | HC-48B2 | Proteobacteria | JH377577.1 |
| 651 | GCA_000234885.1 | 991957 | 666 | Vibrio cholerae HC-33A2 | HC-33A2 | Proteobacteria | JH377410.1 |
| 652 | GCA_000234905.1 | 991956 | 666 | Vibrio cholerae HC-32A1 | HC-32A1 | Proteobacteria | JH377638.1 |
| 653 | GCA_000234925.1 | 991954 | 666 | Vibrio cholerae HC-22A1 | HC-22A1 | Proteobacteria | JH377759.1 |
| 654 | GCA_000234945.1 | 991953 | 666 | Vibrio cholerae HC-21A1 | HC-21A1 | Proteobacteria | JH377863.1 |
| 655 | GCA_000234965.1 | 991952 | 666 | Vibrio cholerae HC-19A1 | HC-19A1 | Proteobacteria | JH377908.1 |
| 656 | GCA_000250855.1 | 1134456 | 666 | Vibrio cholerae IEC224 | IEC224 | Proteobacteria | CP003330.1 |
| 657 | GCA_000279185.1 | 991963 | 666 | Vibrio cholerae HC-42A1 | HC-42A1 | Proteobacteria | JH724360.1 |
| 658 | GCA_000279205.1 | 991974 | 666 | Vibrio cholerae HC-56A2 | HC-56A2 | Proteobacteria | JH724528.1 |
| 659 | GCA_000279245.1 | 991926 | 666 | Vibrio cholerae CP1041(14) | CP1041(14) | Proteobacteria | ALDE01000022.1 |
| 660 | GCA_000279305.1 | 991923 | 666 | Vibrio cholerae CP1032(5) | CP1032(5) | Proteobacteria | ALDA01000015.1 |
| 661 | GCA_000279325.1 | 991925 | 666 | Vibrio cholerae CP1038(11) | CP1038(11) | Proteobacteria | ALDC01000012.1 |
| 662 | GCA_000279345.1 | 991927 | 666 | Vibrio cholerae CP1042(15) | CP1042(15) | Proteobacteria | ALDF01000012.1 |
| 663 | GCA_000279375.1 | 991976 | 666 | Vibrio cholerae HC-57A2 | HC-57A2 | Proteobacteria | JH724633.1 |
| 664 | GCA_000279395.1 | 991929 | 666 | Vibrio cholerae CP1048(21) | CP1048(21) | Proteobacteria | ALDJ01000013.1 |
| 665 | GCA_000279415.1 | 991932 | 666 | Vibrio cholerae HC-20A2 | HC-20A2 | Proteobacteria | ALDL01000012.1 |
| 666 | GCA_000279455.1 | 991940 | 666 | Vibrio cholerae HC-46A1 | HC-46A1 | Proteobacteria | ALDQ01000020.1 |
| 667 | GCA_000279555.1 | 991990 | 666 | Vibrio cholerae CP1030(3) | CP1030(3) | Proteobacteria | JH724671.1 |
| 668 | GCA_000279785.1 | 991995 | 666 | Vibrio cholerae CP1047(20) | CP1047(20) | Proteobacteria | JH724786.1 |
| 669 | GCA_000279955.1 | 992003 | 666 | Vibrio cholerae HC-47A1 | HC-47A1 | Proteobacteria | JH724973.1 |
| 670 | GCA_000281655.1 | 991928 | 666 | Vibrio cholerae CP1046(19) | CP1046(19) | Proteobacteria | ALDH01000023.1 |
| 671 | GCA_000302755.1 | 991962 | 666 | Vibrio cholerae HC-41A1 | HC-41A1 | Proteobacteria | JH941098.1 |
| 672 | GCA_000302775.1 | 991960 | 666 | Vibrio cholerae HC-39A1 | HC-39A1 | Proteobacteria | JH942109.1 |
| 673 | GCA_000302985.1 | 991993 | 666 | Vibrio cholerae CP1040(13) | CP1040(13) | Proteobacteria | JH942548.1 |
| 674 | GCA_000303045.1 | 991994 | 666 | Vibrio cholerae CP1044(17) | CP1044(17) | Proteobacteria | JH942687.1 |
| 675 | GCA_000303065.1 | 991996 | 666 | Vibrio cholerae CP1050(23) | CP1050(23) | Proteobacteria | JH941754.1 |
| 676 | GCA_000303125.1 | 1000951 | 666 | Vibrio cholerae HC-81A2 | HC-81A2 | Proteobacteria | JH941263.1 |
| 677 | GCA_000304755.1 | 991924 | 666 | Vibrio cholerae CP1033(6) | CP1033(6) | Proteobacteria | AJRL01000031.1 |
| 678 | GCA_000304935.2 | 991951 | 666 | Vibrio cholerae HC-17A1 | HC-17A1 | Proteobacteria | JH966643.1 |
| 679 | GCA_000304995.1 | 991968 | 666 | Vibrio cholerae HC-50A2 | HC-50A2 | Proteobacteria | JH967085.1 |
| 680 | GCA_000305075.2 | 992006 | 666 | Vibrio cholerae HC-62A1 | HC-62A1 | Proteobacteria | JH967577.1 |
| 681 | GCA_000305095.2 | 991987 | 666 | Vibrio cholerae HC-77A1 | HC-77A1 | Proteobacteria | JH967738.1 |
| 682 | GCA_000305585.2 | 991958 | 666 | Vibrio cholerae HC-37A1 | HC-37A1 | Proteobacteria | JH973721.1 |
| 683 | GCA_000305625.2 | 992007 | 666 | Vibrio cholerae HC-62B1 | HC-62B1 | Proteobacteria | JH976192.1 |
| 684 | GCA_000305675.2 | 1000952 | 666 | Vibrio cholerae HC-17A2 | HC-17A2 | Proteobacteria | JH974083.1 |
| 685 | GCA_000305695.2 | 992008 | 666 | Vibrio cholerae HC-69A1 | HC-69A1 | Proteobacteria | JH974129.1 |
| 686 | GCA_000318485.2 | 991931 | 666 | Vibrio cholerae HC-7A1 | HC-7A1 | Proteobacteria | AGVA02000011.1 |
| 687 | GCA_000318505.2 | 991936 | 666 | Vibrio cholerae HC-81A1 | HC-81A1 | Proteobacteria | AGVC02000007.1 |
| 688 | GCA_000327105.3 | 991980 | 666 | Vibrio cholerae HC-64A1 | HC-64A1 | Proteobacteria | KB454193.1 |
| 689 | GCA_000327125.3 | 991981 | 666 | Vibrio cholerae HC-65A1 | HC-65A1 | Proteobacteria | KB454294.1 |
| 690 | GCA_000327145.3 | 991982 | 666 | Vibrio cholerae HC-67A1 | HC-67A1 | Proteobacteria | KB454387.1 |
| 691 | GCA_000327165.3 | 991983 | 666 | Vibrio cholerae HC-68A1 | HC-68A1 | Proteobacteria | KB454110.1 |
| 692 | GCA_000327185.3 | 991985 | 666 | Vibrio cholerae HC-71A1 | HC-71A1 | Proteobacteria | KB454017.1 |
| 693 | GCA_000327205.3 | 1000953 | 666 | Vibrio cholerae HC-72A2 | HC-72A2 | Proteobacteria | KB453970.1 |
| 694 | GCA_000327245.3 | 991988 | 666 | Vibrio cholerae HC-80A1 | HC-80A1 | Proteobacteria | KB453739.1 |
| 695 | GCA_000338075.1 | 1224154 | 666 | Vibrio cholerae O1 str. Inaba G4222 | Inaba G4222 | Proteobacteria | CM001785.1 |
| 696 | GCA_000348045.2 | 1095661 | 666 | Vibrio cholerae O1 str. 116059 | 116059 | Proteobacteria | KB661123.1 |
| 697 | GCA_000348105.2 | 1095660 | 666 | Vibrio cholerae O1 str. 95412 | 95412 | Proteobacteria | KB662042.1 |
| 698 | GCA_000348125.2 | 1095645 | 666 | Vibrio cholerae O1 str. AG-7404 | AG-7404 | Proteobacteria | KB662659.1 |
| 699 | GCA_000348145.2 | 1095644 | 666 | Vibrio cholerae O1 str. AG-8040 | AG-8040 | Proteobacteria | KB663097.1 |
| 700 | GCA_000348165.2 | 1095651 | 666 | Vibrio cholerae O1 str. EC-0009 | EC-0009 | Proteobacteria | KB663518.1 |
| 701 | GCA_000348185.2 | 1095652 | 666 | Vibrio cholerae O1 str. EC-0012 | EC-0012 | Proteobacteria | KB661760.1 |
| 702 | GCA_000348205.2 | 1095653 | 666 | Vibrio cholerae O1 str. EC-0027 | EC-0027 | Proteobacteria | KB663429.1 |
| 703 | GCA_000348225.2 | 1095654 | 666 | Vibrio cholerae O1 str. EC-0051 | EC-0051 | Proteobacteria | KB661629.1 |
| 704 | GCA_000348245.2 | 1095640 | 666 | Vibrio cholerae O1 str. EDC-020 | EDC-020 | Proteobacteria | KB663379.1 |
| 705 | GCA_000348265.2 | 1095641 | 666 | Vibrio cholerae O1 str. EDC-022 | EDC-022 | Proteobacteria | KB663300.1 |
| 706 | GCA_000348285.2 | 1095646 | 666 | Vibrio cholerae O1 str. EM-1536 | EM-1536 | Proteobacteria | KB662939.1 |
| 707 | GCA_000348305.2 | 1095647 | 666 | Vibrio cholerae O1 str. EM-1546 | EM-1546 | Proteobacteria | KB663192.1 |
| 708 | GCA_000348325.2 | 1095648 | 666 | Vibrio cholerae O1 str. EM-1626 | EM-1626 | Proteobacteria | KB662879.1 |
| 709 | GCA_000348365.1 | 1095650 | 666 | Vibrio cholerae O1 str. EM-1727 | EM-1727 | Proteobacteria | KB662387.1 |
| 710 | GCA_000348385.2 | 1095655 | 666 | Vibrio cholerae O1 str. NHCC-004A | NHCC-004A | Proteobacteria | KB662555.1 |
| 711 | GCA_000348405.2 | 1095656 | 666 | Vibrio cholerae O1 str. NHCC-006C | NHCC-006C | Proteobacteria | KB662604.1 |
| 712 | GCA_000348445.2 | 1095658 | 666 | Vibrio cholerae O1 str. NHCC-010F | NHCC-010F | Proteobacteria | KB662324.1 |
| 713 | GCA_000348465.2 | 1095642 | 666 | Vibrio cholerae O1 str. Nep-21106 | Nep-21106 | Proteobacteria | KB661614.1 |
| 714 | GCA_000348485.2 | 1095643 | 666 | Vibrio cholerae O1 str. Nep-21113 | Nep-21113 | Proteobacteria | KB662142.1 |
| 715 | GCA_000348505.2 | 1095639 | 666 | Vibrio cholerae O1 str. PCS-023 | PCS-023 | Proteobacteria | KB661938.1 |
| 716 | GCA_000569115.2 | 1095638 | 666 | Vibrio cholerae O1 str. PCS-022 | PCS-022 | Proteobacteria | KK037973.1 |
| 717 | GCA_000705295.1 | 686 | 666 | Vibrio cholerae O1 biovar El Tor | M-1293 | Proteobacteria | JFFW01000006.1 |
| 718 | GCA_000710155.1 | 1343738 | 666 | Vibrio cholerae 2012EL-1759 | 2012EL-1759 | Proteobacteria | JNEW01000011.1 |
| 719 | GCA_000710445.1 | 686 | 666 | Vibrio cholerae O1 biovar El Tor | Ogawa RND19188 | Proteobacteria | JNGU01000059.1 |
| 720 | GCA_000710455.1 | 686 | 666 | Vibrio cholerae O1 biovar El Tor | Ogawa RND19191 | Proteobacteria | JNGT01000078.1 |
| 721 | GCA_000736795.1 | 666 | 666 | Vibrio cholerae | 5473-62 | Proteobacteria | JIDI01000041.1 |
| 722 | GCA_000736855.1 | 666 | 666 | Vibrio cholerae | 1311-69 | Proteobacteria | JIDJ01000055.1 |
| 723 | GCA_000736865.1 | 666 | 666 | Vibrio cholerae | NIH41 | Proteobacteria | JIDH01000006.1 |
| 724 | GCA_000736925.1 | 666 | 666 | Vibrio cholerae | 981-75 | Proteobacteria | JIDM01000006.1 |
| 725 | GCA_000753725.1 | 686 | 666 | Vibrio cholerae O1 biovar El Tor | MAK676 | Proteobacteria | JRBD01000007.1 |
| 726 | GCA_000765415.1 | 666 | 666 | Vibrio cholerae | 2012EL-2176 | Proteobacteria | CP007634.1 |
| 727 | GCA_000786335.1 | 666 | 666 | Vibrio cholerae | 81 | Proteobacteria | JRQM01000007.1 |
| 728 | GCA_000786345.1 | 666 | 666 | Vibrio cholerae | 3265/80 | Proteobacteria | JRQL01000006.1 |
| 729 | GCA_000829215.1 | 1420885 | 666 | Vibrio cholerae MS6 | MS6 | Proteobacteria | AP014524.1 |
| 730 | GCA_000939665.1 | 686 | 666 | Vibrio cholerae O1 biovar El Tor | MAK97 | Proteobacteria | JZCB01000007.1 |
| 731 | GCA_000953755.1 | 666 | 666 | Vibrio cholerae | InDRE 4354 | Proteobacteria | JZUA01000014.1 |
| 732 | GCA_000953775.1 | 666 | 666 | Vibrio cholerae | InDRE 4262 | Proteobacteria | JZUB01000015.1 |
| 733 | GCA_000960915.1 | 686 | 666 | Vibrio cholerae O1 biovar El Tor | M1429 | Proteobacteria | LAEM01000007.1 |
| 734 | GCA_000961975.1 | 666 | 666 | Vibrio cholerae | D-35 | Proteobacteria | LAED01000005.1 |
| 735 | GCA_000963555.1 | 686 | 666 | Vibrio cholerae O1 biovar El Tor | FJ147 | Proteobacteria | CP009042.1 |
| 736 | GCA_000965285.1 | 686 | 666 | Vibrio cholerae O1 biovar El Tor | R17644 | Proteobacteria | JRTW01000033.1 |
| 737 | GCA_000966385.1 | 666 | 666 | Vibrio cholerae | M818 | Proteobacteria | LAHM01000006.1 |
| 738 | GCA_001029975.1 | 666 | 666 | Vibrio cholerae | YN2011004 | Proteobacteria | KQ087383.1 |
| 739 | GCA_001030015.1 | 666 | 666 | Vibrio cholerae | YN97083 | Proteobacteria | KQ087448.1 |
| 740 | GCA_001030035.1 | 666 | 666 | Vibrio cholerae | YN89004 | Proteobacteria | KQ087512.1 |
| 741 | GCA_001045415.1 | 666 | 666 | Vibrio cholerae | TSY216 | Proteobacteria | CP007653.1 |
| 742 | GCA_001246285.1 | 666 | 666 | Vibrio cholerae | A346(2) | Proteobacteria | CWQG01000013.1 |
| 743 | GCA_001247245.1 | 666 | 666 | Vibrio cholerae | 4679 | Proteobacteria | CWOC01000002.1 |
| 744 | GCA_001247525.1 | 666 | 666 | Vibrio cholerae | A346(1) | Proteobacteria | CWSM01000004.1 |
| 745 | GCA_001247835.1 | 666 | 666 | Vibrio cholerae | 1627 | Proteobacteria | CWNK01000005.1 |
| 746 | GCA_001247885.1 | 666 | 666 | Vibrio cholerae | IDHO1_726 | Proteobacteria | CWQN01000008.1 |
| 747 | GCA_001248135.1 | 666 | 666 | Vibrio cholerae | A186 | Proteobacteria | CWPM01000020.1 |
| 748 | GCA_001248195.1 | 666 | 666 | Vibrio cholerae | A60 | Proteobacteria | CWOY01000004.1 |
| 749 | GCA_001248235.1 | 666 | 666 | Vibrio cholerae | A217 | Proteobacteria | CWPV01000006.1 |
| 750 | GCA_001248365.1 | 666 | 666 | Vibrio cholerae | A397 | Proteobacteria | CWQB01000004.1 |
| 751 | GCA_001248465.1 | 666 | 666 | Vibrio cholerae | V212-1 | Proteobacteria | CWPH01000003.1 |
| 752 | GCA_001248505.1 | 666 | 666 | Vibrio cholerae | 4519 | Proteobacteria | CWRZ01000004.1 |
| 753 | GCA_001248645.1 | 666 | 666 | Vibrio cholerae | 6214 | Proteobacteria | CWQT01000002.1 |
| 754 | GCA_001248865.1 | 666 | 666 | Vibrio cholerae | A193 | Proteobacteria | CWPN01000010.1 |
| 755 | GCA_001248905.1 | 666 | 666 | Vibrio cholerae | A70 | Proteobacteria | CWOM01000009.1 |
| 756 | GCA_001248945.1 | 666 | 666 | Vibrio cholerae | A213 | Proteobacteria | CWPS01000006.1 |
| 757 | GCA_001249085.1 | 666 | 666 | Vibrio cholerae | MBRN14 | Proteobacteria | CWNR01000004.1 |
| 758 | GCA_001249315.1 | 666 | 666 | Vibrio cholerae | 4322 | Proteobacteria | CWOX01000006.1 |
| 759 | GCA_001249515.1 | 666 | 666 | Vibrio cholerae | 6191 | Proteobacteria | CWQQ01000006.1 |
| 760 | GCA_001249715.1 | 666 | 666 | Vibrio cholerae | GP152 | Proteobacteria | CWQK01000006.1 |
| 761 | GCA_001249815.1 | 666 | 666 | Vibrio cholerae | 7682 | Proteobacteria | CWNO01000007.1 |
| 762 | GCA_001249995.1 | 666 | 666 | Vibrio cholerae | A177 | Proteobacteria | CWNQ01000004.1 |
| 763 | GCA_001250035.1 | 666 | 666 | Vibrio cholerae | GP145 | Proteobacteria | CWQL01000008.1 |
| 764 | GCA_001250195.1 | 666 | 666 | Vibrio cholerae | MJ1485 | Proteobacteria | CWNZ01000023.1 |
| 765 | GCA_001250235.1 | 666 | 666 | Vibrio cholerae | A19 | Proteobacteria | CWSH01000005.1 |
| 766 | GCA_001250435.1 | 666 | 666 | Vibrio cholerae | 4642 | Proteobacteria | CWPE01000005.1 |
| 767 | GCA_001250455.1 | 666 | 666 | Vibrio cholerae | A32 | Proteobacteria | CWNM01000004.1 |
| 768 | GCA_001250615.1 | 666 | 666 | Vibrio cholerae | A488(2) | Proteobacteria | CWOA01000007.1 |
| 769 | GCA_001250795.1 | 666 | 666 | Vibrio cholerae | MBN17 | Proteobacteria | CWNW01000004.1 |
| 770 | GCA_001250935.1 | 666 | 666 | Vibrio cholerae | A61 | Proteobacteria | CWOS01000008.1 |
| 771 | GCA_001251155.1 | 666 | 666 | Vibrio cholerae | A130 | Proteobacteria | CWNY01000012.1 |
| 772 | GCA_001251415.1 | 666 | 666 | Vibrio cholerae | A487(2) | Proteobacteria | CWQX01000001.1 |
| 773 | GCA_001251435.1 | 666 | 666 | Vibrio cholerae | 7687 | Proteobacteria | CWRG01000008.1 |
| 774 | GCA_001251495.1 | 666 | 666 | Vibrio cholerae | GP16 | Proteobacteria | CWQE01000007.1 |
| 775 | GCA_001251595.1 | 666 | 666 | Vibrio cholerae | A483 | Proteobacteria | CWQA01000003.1 |
| 776 | GCA_001251935.1 | 666 | 666 | Vibrio cholerae | 6194 | Proteobacteria | CWQW01000017.1 |
| 777 | GCA_001251975.1 | 666 | 666 | Vibrio cholerae | 6212 | Proteobacteria | CWQV01000004.1 |
| 778 | GCA_001252055.1 | 666 | 666 | Vibrio cholerae | 4551 | Proteobacteria | CWRK01000003.1 |
| 779 | GCA_001252075.1 | 666 | 666 | Vibrio cholerae | 4121 | Proteobacteria | CWRP01000009.1 |
| 780 | GCA_001252495.1 | 666 | 666 | Vibrio cholerae | A18 | Proteobacteria | CWSJ01000005.1 |
| 781 | GCA_001252675.1 | 666 | 666 | Vibrio cholerae | V5 | Proteobacteria | CWNT01000007.1 |
| 782 | GCA_001252775.1 | 666 | 666 | Vibrio cholerae | 6215 | Proteobacteria | CWRD01000009.1 |
| 783 | GCA_001252855.1 | 666 | 666 | Vibrio cholerae | 4111 | Proteobacteria | CWOZ01000011.1 |
| 784 | GCA_001252875.1 | 666 | 666 | Vibrio cholerae | A152 | Proteobacteria | CWOB01000008.1 |
| 785 | GCA_001252895.1 | 666 | 666 | Vibrio cholerae | 4585 | Proteobacteria | CWRS01000004.1 |
| 786 | GCA_001253035.1 | 666 | 666 | Vibrio cholerae | 1346 | Proteobacteria | CWRJ01000005.1 |
| 787 | GCA_001253055.1 | 666 | 666 | Vibrio cholerae | 4600 | Proteobacteria | CWRW01000004.1 |
| 788 | GCA_001253155.1 | 666 | 666 | Vibrio cholerae | A154 | Proteobacteria | CWNX01000009.1 |
| 789 | GCA_001253175.1 | 666 | 666 | Vibrio cholerae | A184 | Proteobacteria | CWOO01000008.1 |
| 790 | GCA_001253235.1 | 666 | 666 | Vibrio cholerae | A29 | Proteobacteria | CWSI01000004.1 |
| 791 | GCA_001253295.1 | 666 | 666 | Vibrio cholerae | A185 | Proteobacteria | CWOK01000008.1 |
| 792 | GCA_001253315.1 | 666 | 666 | Vibrio cholerae | GP140 | Proteobacteria | CWRA01000011.1 |
| 793 | GCA_001253435.1 | 666 | 666 | Vibrio cholerae | A51 | Proteobacteria | CWOW01000014.1 |
| 794 | GCA_001253495.1 | 666 | 666 | Vibrio cholerae | A111 | Proteobacteria | CWOV01000006.1 |
| 795 | GCA_001253555.1 | 666 | 666 | Vibrio cholerae | A279 | Proteobacteria | CWQC01000021.1 |
| 796 | GCA_001253575.1 | 666 | 666 | Vibrio cholerae | GP8 | Proteobacteria | CWNN01000007.1 |
| 797 | GCA_001253615.1 | 666 | 666 | Vibrio cholerae | A219 | Proteobacteria | CWPQ01000002.1 |
| 798 | GCA_001253695.1 | 666 | 666 | Vibrio cholerae | A31 | Proteobacteria | CWSK01000004.1 |
| 799 | GCA_001253755.1 | 666 | 666 | Vibrio cholerae | VC51 | Proteobacteria | CWOI01000007.1 |
| 800 | GCA_001253835.1 | 666 | 666 | Vibrio cholerae | A49 | Proteobacteria | CWPI01000004.1 |
| 801 | GCA_001254055.1 | 666 | 666 | Vibrio cholerae | A4 | Proteobacteria | CWSC01000005.1 |
| 802 | GCA_001254255.1 | 666 | 666 | Vibrio cholerae | A481 | Proteobacteria | CWQD01000029.1 |
| 803 | GCA_001254335.1 | 666 | 666 | Vibrio cholerae | 4784 | Proteobacteria | CWRV01000004.1 |
| 804 | GCA_001254355.1 | 666 | 666 | Vibrio cholerae | MG116226 | Proteobacteria | CWSQ01000005.1 |
| 805 | GCA_001254435.1 | 666 | 666 | Vibrio cholerae | GP160 | Proteobacteria | CWSB01000005.1 |
| 806 | GCA_001254535.1 | 666 | 666 | Vibrio cholerae | A59 | Proteobacteria | CWOG01000006.1 |
| 807 | GCA_001254575.1 | 666 | 666 | Vibrio cholerae | A103 | Proteobacteria | CWOP01000008.1 |
| 808 | GCA_001254635.1 | 666 | 666 | Vibrio cholerae | 4623 | Proteobacteria | CWRM01000004.1 |
| 809 | GCA_001254655.1 | 666 | 666 | Vibrio cholerae | A10 | Proteobacteria | CWSF01000005.1 |
| 810 | GCA_001254675.1 | 666 | 666 | Vibrio cholerae | A5 | Proteobacteria | CWSE01000005.1 |
| 811 | GCA_001254895.1 | 666 | 666 | Vibrio cholerae | MG116025 | Proteobacteria | CWNS01000006.1 |
| 812 | GCA_001254915.1 | 666 | 666 | Vibrio cholerae | A180 | Proteobacteria | CWOR01000008.1 |
| 813 | GCA_001254955.1 | 666 | 666 | Vibrio cholerae | 6197 | Proteobacteria | CWRB01000040.1 |
| 814 | GCA_001255155.1 | 666 | 666 | Vibrio cholerae | A22 | Proteobacteria | CWNL01000007.1 |
| 815 | GCA_001255295.1 | 666 | 666 | Vibrio cholerae | A200 | Proteobacteria | CWPW01000009.1 |
| 816 | GCA_001255575.1 | 666 | 666 | Vibrio cholerae | A6 | Proteobacteria | CWSG01000004.1 |
| 817 | GCA_001255835.1 | 666 | 666 | Vibrio cholerae | 4536 | Proteobacteria | CWSA01000001.1 |
| 818 | GCA_001255915.1 | 666 | 666 | Vibrio cholerae | 7685 | Proteobacteria | CWRI01000009.1 |
| 819 | GCA_001256015.1 | 666 | 666 | Vibrio cholerae | 4663 | Proteobacteria | CWOD01000006.1 |
| 820 | GCA_001256355.1 | 666 | 666 | Vibrio cholerae | PRL5 | Proteobacteria | CWQI01000001.1 |
| 821 | GCA_001256655.1 | 666 | 666 | Vibrio cholerae | A209 | Proteobacteria | CWPU01000001.1 |
| 822 | GCA_001256675.1 | 666 | 666 | Vibrio cholerae | A241 | Proteobacteria | CWPX01000008.1 |
| 823 | GCA_001256835.1 | 666 | 666 | Vibrio cholerae | A231 | Proteobacteria | CWPP01000001.1 |
| 824 | GCA_001257035.1 | 666 | 666 | Vibrio cholerae | 4110 | Proteobacteria | CWOT01000002.1 |
| 825 | GCA_001257075.1 | 666 | 666 | Vibrio cholerae | A488(1) | Proteobacteria | CWSN01000007.1 |
| 826 | GCA_001257255.1 | 666 | 666 | Vibrio cholerae | V109 | Proteobacteria | CWOJ01000008.1 |
| 827 | GCA_001257835.1 | 666 | 666 | Vibrio cholerae | 6193 | Proteobacteria | CWQZ01000004.1 |
| 828 | GCA_001257895.1 | 666 | 666 | Vibrio cholerae | 4593 | Proteobacteria | CWRL01000004.1 |
| 829 | GCA_001258495.1 | 666 | 666 | Vibrio cholerae | 4656 | Proteobacteria | CWOQ01000004.1 |
| 830 | GCA_001258535.1 | 666 | 666 | Vibrio cholerae | 7686 | Proteobacteria | CWRH01000019.1 |
| 831 | GCA_001258555.1 | 666 | 666 | Vibrio cholerae | 4488 | Proteobacteria | CWRU01000004.1 |
| 832 | GCA_001258995.1 | 666 | 666 | Vibrio cholerae | 4646 | Proteobacteria | CWRX01000004.1 |
| 833 | GCA_001259055.1 | 666 | 666 | Vibrio cholerae | 4113 | Proteobacteria | CWRQ01000003.1 |
| 834 | GCA_001259135.1 | 666 | 666 | Vibrio cholerae | 4605 | Proteobacteria | CWON01000005.1 |
| 835 | GCA_001259235.1 | 666 | 666 | Vibrio cholerae | 4552 | Proteobacteria | CWRT01000003.1 |
| 836 | GCA_001259315.1 | 666 | 666 | Vibrio cholerae | A131 | Proteobacteria | CWOL01000002.1 |
| 837 | GCA_001259475.1 | 666 | 666 | Vibrio cholerae | 6210 | Proteobacteria | CWQO01000008.1 |
| 838 | GCA_001259495.1 | 666 | 666 | Vibrio cholerae | A76 | Proteobacteria | CWOF01000008.1 |
| 839 | GCA_001259555.1 | 666 | 666 | Vibrio cholerae | A46 | Proteobacteria | CWOH01000007.1 |
| 840 | GCA_001259575.1 | 666 | 666 | Vibrio cholerae | A390 | Proteobacteria | CWQF01000001.1 |
| 841 | GCA_001259635.1 | 666 | 666 | Vibrio cholerae | A68 | Proteobacteria | CWNV01000008.1 |
| 842 | GCA_001259715.1 | 666 | 666 | Vibrio cholerae | 4538 | Proteobacteria | CWRO01000004.1 |
| 843 | GCA_001259795.1 | 666 | 666 | Vibrio cholerae | A389 | Proteobacteria | CWQH01000016.1 |
| 844 | GCA_001259915.1 | 666 | 666 | Vibrio cholerae | A155 | Proteobacteria | CWOE01000008.1 |
| 845 | GCA_001259935.1 | 666 | 666 | Vibrio cholerae | A109 | Proteobacteria | CWPD01000024.1 |
| 846 | GCA_001260175.1 | 666 | 666 | Vibrio cholerae | 4662 | Proteobacteria | CWRY01000001.1 |
| 847 | GCA_001260295.1 | 666 | 666 | Vibrio cholerae | 1362 | Proteobacteria | CWRR01000004.1 |
| 848 | GCA_001260335.1 | 666 | 666 | Vibrio cholerae | 4339 | Proteobacteria | CWRN01000004.1 |
| 849 | GCA_001260915.1 | 666 | 666 | Vibrio cholerae | A66 | Proteobacteria | CWOU01000006.1 |
| 850 | GCA_001260995.1 | 666 | 666 | Vibrio cholerae | A27 | Proteobacteria | CWSD01000004.1 |
| 851 | GCA_001261075.1 | 666 | 666 | Vibrio cholerae | PRL64 | Proteobacteria | CWQM01000006.1 |
| 852 | GCA_001261095.1 | 666 | 666 | Vibrio cholerae | A482 | Proteobacteria | CWPZ01000016.1 |
| 853 | GCA_001261135.1 | 666 | 666 | Vibrio cholerae | A245 | Proteobacteria | CWPY01000019.1 |
| 854 | GCA_001261515.1 | 666 | 666 | Vibrio cholerae | A201 | Proteobacteria | CWPT01000008.1 |
| 855 | GCA_001261535.1 | 666 | 666 | Vibrio cholerae | A487(1) | Proteobacteria | CWSP01000004.1 |
| 856 | GCA_001261555.1 | 666 | 666 | Vibrio cholerae | 6201 | Proteobacteria | CWQP01000004.1 |
| 857 | GCA_001281585.1 | 686 | 666 | Vibrio cholerae O1 biovar El Tor | 31 | Proteobacteria | LIFF01000004.1 |
| 858 | GCA_001281595.1 | 686 | 666 | Vibrio cholerae O1 biovar El Tor | 39 | Proteobacteria | LIFG01000007.1 |
| 859 | GCA_001281615.1 | 686 | 666 | Vibrio cholerae O1 biovar El Tor | 43 | Proteobacteria | LIFH01000031.1 |
| 860 | GCA_001281665.1 | 686 | 666 | Vibrio cholerae O1 biovar El Tor | 56 | Proteobacteria | LIFI01000004.1 |
| 861 | GCA_001282605.1 | 666 | 666 | Vibrio cholerae | EC-051 | Proteobacteria | LFZZ01000030.1 |
| 862 | GCA_001318185.1 | 1433144 | 666 | Vibrio cholerae O1 str. KW3 | KW3 | Proteobacteria | CP006947.1 |
| 863 | GCA_001471455.1 | 666 | 666 | Vibrio cholerae | ATCC 11629 | Proteobacteria | LOSM01000005.1 |
| 864 | GCA_001515095.1 | 666 | 666 | Vibrio cholerae | ME-7 | Proteobacteria | LQBW01000005.1 |
| 865 | GCA_001517845.1 | 686 | 666 | Vibrio cholerae O1 biovar El Tor | M1275 | Proteobacteria | LRAF01000011.1 |
| 866 | GCA_001517855.1 | 686 | 666 | Vibrio cholerae O1 biovar El Tor | M1275D | Proteobacteria | LRAG01000003.1 |
| 867 | GCA_001521835.1 | 666 | 666 | Vibrio cholerae | M888 | Proteobacteria | LRBH01000022.1 |
| 868 | GCA_001525525.1 | 666 | 666 | Vibrio cholerae | ATCC 25874 | Proteobacteria | LRIK01000002.1 |
| 869 | GCA_001543465.1 | 686 | 666 | Vibrio cholerae O1 biovar El Tor | Inaba PIC018 | Proteobacteria | LQHM01000074.1 |
| 870 | GCA_000260675.1 | 1095743 | 736 | Haemophilus paraphrohaemolyticus HK411 | HK411 | Proteobacteria | AJMU01000074.1 |
| 871 | GCA_000771955.1 | 750 | 750 | Gallibacterium anatis | F0003406 | Proteobacteria | JPXV01000009.1 |
| 872 | GCA_000772275.1 | 750 | 750 | Gallibacterium anatis | 10672/9 | Proteobacteria | JPTU01000008.1 |
| 873 | GCA_000772385.1 | 1396511 | 750 | Gallibacterium anatis 7990 | 7990 | Proteobacteria | JPJL01000011.1 |
| 874 | GCA_000024665.1 | 59374 | 833 | Fibrobacter succinogenes subsp. succinogenes S85 | S85 | Fibrobacteres | CP001792.1 |
| 875 | GCA_000146505.1 | 59374 | 833 | Fibrobacter succinogenes subsp. succinogenes S85 | S85 | Fibrobacteres | CP002158.1 |
| 876 | GCA_000158275.2 | 457405 | 851 | Fusobacterium nucleatum subsp. animalis 7_1 | 7_1 | Fusobacteria | CP007062.1 |
| 877 | GCA_000234075.2 | 693991 | 851 | Fusobacterium nucleatum subsp. polymorphum F0401 | F0401 | Fusobacteria | ADDB02000001.1 |
| 878 | GCA_000479285.1 | 1204474 | 851 | Fusobacterium nucleatum CTI-1 | CTI-1 | Fusobacteria | KI518459.1 |
| 879 | GCA_000158215.3 | 469599 | 860 | Fusobacterium periodonticum 2_1_31 | 2_1_31 | Fusobacteria | KN173678.1 |
| 880 | GCA_000160475.1 | 546275 | 860 | Fusobacterium periodonticum ATCC 33693 | ATCC 33693 | Fusobacteria | GG665893.1 |
| 881 | GCA_000015485.1 | 391774 | 881 | Desulfovibrio vulgaris DP4 | DP4 | Proteobacteria | CP000527.1 |
| 882 | GCA_000025305.1 | 591001 | 905 | Acidaminococcus fermentans DSM 20731 | DSM 20731 | Firmicutes | CP001859.1 |
| 883 | GCA_000021865.1 | 272942 | 1061 | Rhodobacter capsulatus SB 1003 | SB 1003 | Proteobacteria | CP001312.1 |
| 884 | GCA_000505785.1 | 1415160 | 1061 | Rhodobacter capsulatus DE442 | DE442 | Proteobacteria | AYPR01000015.1 |
| 885 | GCA_000506425.2 | 1415162 | 1061 | Rhodobacter capsulatus B6 | B6 | Proteobacteria | AYQA01000010.1 |
| 886 | GCA_000506565.2 | 1415163 | 1061 | Rhodobacter capsulatus R121 | R121 | Proteobacteria | AYQC01000013.1 |
| 887 | GCA_000506965.2 | 1415161 | 1061 | Rhodobacter capsulatus Y262 | Y262 | Proteobacteria | AYQB01000018.1 |
| 888 | GCA_000341585.2 | 317619 | 1223 | Prochlorothrix hollandica PCC 9006 | | Cyanobacteria | AJTX02000004.1 |
| 889 | GCA_000740735.1 | 1229 | 1229 | Nitrosococcus oceani | AFC132 | Proteobacteria | JPFN01000044.1 |
| 890 | GCA_001183895.1 | 1422 | 1422 | Geobacillus stearothermophilus | A1 | Firmicutes | LDNS01000331.1 |
| 891 | GCA_001183915.1 | 1422 | 1422 | Geobacillus stearothermophilus | P3 | Firmicutes | LDNT01000009.1 |
| 892 | GCA_001587395.1 | 1422 | 1422 | Geobacillus stearothermophilus | B4114 | Firmicutes | LQYY01000004.1 |
| 893 | GCA_000415905.1 | 1337888 | 1462 | Geobacillus kaustophilus GBlys | GBlys | Firmicutes | BASG01000012.1 |
| 894 | GCA_000353265.2 | 1198630 | 1516 | Thermoanaerobacter thermohydrosulfuricus WC1 | WC1 | Firmicutes | KB731278.1 |
| 895 | GCA_000219125.2 | 1009370 | 2374 | Acetonema longum DSM 6540 | DSM 6540 | Firmicutes | AFGF01000017.1 |
| 896 | GCA_000300715.1 | 1161912 | 2421 | Thermosipho africanus H17ap60334 | H17ap60334 | Thermotogae | AJIP01000035.1 |
| 897 | GCA_000284255.1 | 983917 | 28068 | Rubrivivax gelatinosus IL144 | IL144 | Proteobacteria | AP012320.1 |
| 898 | GCA_001467525.1 | 28082 | 28082 | Legionella anisa | WA-316-C3 | Proteobacteria | LNXS01000010.1 |
| 899 | GCA_000178275.1 | 702443 | 28116 | Bacteroides ovatus SD CMC 3f | SD CMC 3f | Bacteroidetes | ADMO01000148.1 |
| 900 | GCA_000017665.1 | 290339 | 28141 | Cronobacter sakazakii ATCC BAA-894 | ATCC BAA-894 | Proteobacteria | CP000783.1 |
| 901 | GCA_000214745.2 | 930780 | 28141 | Cronobacter sakazakii E899 | E899 | Proteobacteria | AFMO01000120.1 |
| 902 | GCA_000316155.1 | 1208664 | 28141 | Cronobacter sakazakii 696 | 696 | Proteobacteria | CALF01000071.1 |
| 903 | GCA_000319595.1 | 1208663 | 28141 | Cronobacter sakazakii 701 | 701 | Proteobacteria | CALE01000616.1 |
| 904 | GCA_000723765.1 | 1238450 | 28173 | Vibrio nigripulchritudo SOn1 | SOn1 | Proteobacteria | LK391965.1 |
| 905 | GCA_000307105.1 | 651182 | 28223 | Desulfobacula toluolica Tol2 | Tol2 | Proteobacteria | FO203503.1 |
| 906 | GCA_000757225.1 | 28450 | 28450 | Burkholderia pseudomallei | BEC | Proteobacteria | KN150953.1 |
| 907 | GCA_000773415.1 | 1435374 | 28450 | Burkholderia pseudomallei MSHR7500 | MSHR7500 | Proteobacteria | JREN01000128.1 |
| 908 | GCA_000774635.1 | 1434195 | 28450 | Burkholderia pseudomallei MSHR4303 | MSHR4303 | Proteobacteria | KN323039.1 |
| 909 | GCA_000775025.1 | 1437004 | 28450 | Burkholderia pseudomallei MSHR3709 | MSHR3709 | Proteobacteria | KN323107.1 |
| 910 | GCA_000775045.1 | 1435993 | 28450 | Burkholderia pseudomallei MSHR2138 | MSHR2138 | Proteobacteria | KN323181.1 |
| 911 | GCA_001202135.1 | 28450 | 28450 | Burkholderia pseudomallei | P166/04 | Proteobacteria | CSLD01000016.1 |
| 912 | GCA_001448845.1 | 1240693 | 28901 | Salmonella enterica subsp. enterica serovar Rubislaw str. | SA20030553 | Proteobacteria | LHTG01000011.1 |
| | | | | SA20030553 | | | |
| 913 | GCA_000189775.3 | 858215 | 29329 | Thermoanaerobacterium xylanolyticum LX-11 | LX-11 | Firmicutes | CP002739.1 |
| 914 | GCA_000369085.1 | 1217986 | 29430 | Acinetobacter haemolyticus NIPH 261 | NIPH 261 | Proteobacteria | KB849819.1 |
| 915 | GCA_000015745.1 | 420246 | 33940 | Geobacillus thermodenitrificans NG80-2 | NG80-2 | Firmicutes | CP000557.1 |
| 916 | GCA_000016185.1 | 414684 | 34018 | Rhodospirillum centenum SW | SW; ATCC 51521 | Proteobacteria | CP000613.2 |
| 917 | GCA_000023345.1 | 543728 | 34073 | Variovorax paradoxus S110 | S110 | Proteobacteria | CP001635.1 |
| 918 | GCA_000214275.3 | 686340 | 39775 | Methylomicrobium album BG8 | BG8 | Proteobacteria | CM001475.1 |
| 919 | GCA_000175675.1 | 596318 | 40216 | Acinetobacter radioresistens SK82 | SK82 | Proteobacteria | ACVR01000061.1 |
| 920 | GCA_000367985.1 | 40373 | 40373 | Acinetobacter sp. CIP-A165 | CIP A165 | Proteobacteria | KB849201.1 |
| 921 | GCA_001273775.1 | 42253 | 42253 | Nitrospira moscoviensis | NSP M-1 | Nitrospirae | CP011801.1 |
| 922 | GCA_000328625.1 | 748449 | 42422 | Halobacteroides halobius DSM 5150 | DSM 5150 | Firmicutes | CP003359.1 |
| 923 | GCA_000154785.2 | 391595 | 42443 | Roseobacter litoralis Och 149 | Och 149 | Proteobacteria | CP002623.1 |
| 924 | GCA_000350165.1 | 1293054 | 43595 | Halanaerobium saccharolyticum subsp. saccharolyticum | type strain:DSM 6643 | Firmicutes | CAUI01000005.1 |
| | | | | DSM 6643 | | | |
| 925 | GCA_000238375.3 | 1117314 | 43655 | Pseudoalteromonas citrea DSM 8771 | NCIMB 1889 | Proteobacteria | AHBZ02000147.1 |
| 926 | GCA_001298525.1 | 45610 | 45610 | Psychrobacter urativorans | R10.10B | Proteobacteria | CP012678.1 |
| 927 | GCA_000147695.3 | 697303 | 46354 | Thermoanaerobacter wiegelii Rt8.B1 | Rt8.B1 | Firmicutes | CP002991.1 |
| 928 | GCA_000186425.1 | 887898 | 47671 | Lautropia mirabilis ATCC 51599 | ATCC 51599 | Proteobacteria | GL636062.1 |
| 929 | GCA_001432715.1 | 48296 | 48296 | Acinetobacter pittii | ABBL005 | Proteobacteria | LLCL01000049.1 |
| 930 | GCA_000219855.1 | 1051632 | 53633 | Sulfobacillus acidophilus TPY | TPY | Firmicutes | CP002901.1 |
| 931 | GCA_000237975.1 | 679936 | 53633 | Sulfobacillus acidophilus DSM 10332 | DSM 10332 | Firmicutes | CP003179.1 |
| 932 | GCA_000749925.1 | 55207 | 55207 | Pectobacterium betavasculorum | NCPPB 2793 | Proteobacteria | JQHL01000017.1 |
| 933 | GCA_001005725.1 | 56193 | 56193 | Sphingobium chungbukense | DJ77 | Proteobacteria | LBIC01000001.1 |
| 934 | GCA_001529365.1 | 56812 | 56812 | Shewanella frigidimarina | Ag06-30 | Proteobacteria | LRDC01000001.1 |
| 935 | GCA_000958425.1 | 61645 | 61645 | Enterobacter asburiae | 42192 | Proteobacteria | JZYJ01000004.1 |
| 936 | GCA_001011855.1 | 61645 | 61645 | Enterobacter asburiae | GN02073 | Proteobacteria | LDCE01000036.1 |
| 937 | GCA_001011955.1 | 61645 | 61645 | Enterobacter asburiae | GN02208 | Proteobacteria | LDCK01000037.1 |
| 938 | GCA_001023015.1 | 61645 | 61645 | Enterobacter asburiae | GN04222 | Proteobacteria | LECX01000031.1 |
| 939 | GCA_001471995.1 | 61645 | 61645 | Enterobacter asburiae | SMART_269 | Proteobacteria | LPQP01000010.1 |
| 940 | GCA_001472305.1 | 61645 | 61645 | Enterobacter asburiae | SMART_454 | Proteobacteria | LPPZ01000027.1 |
| 941 | GCA_001472715.1 | 61645 | 61645 | Enterobacter asburiae | SMART_763 | Proteobacteria | LPPF01000080.1 |
| 942 | GCA_001022135.1 | 61648 | 61648 | Kluyvera intermedia | CAV1151 | Proteobacteria | CP011602.1 |
| 943 | GCA_000317635.1 | 65093 | 65093 | Halothece sp. PCC 7418 | PCC 7418 | Cyanobacteria | CP003945.1 |
| 944 | GCA_000021825.1 | 65393 | 65393 | Cyanothece sp. PCC 7424 | PCC 7424 | Cyanobacteria | CP001291.1 |
| 945 | GCA_000832305.1 | 68895 | 68895 | Cupriavidus basilensis | 4G11 | Proteobacteria | CP010537.1 |
| 946 | GCA_000169135.1 | 313624 | 70799 | Nodularia spumigena CCY9414 | CCY9414 | Cyanobacteria | AAVW01000117.1 |
| 947 | GCA_000340565.3 | 313624 | 70799 | Nodularia spumigena CCY9414 | CCY9414 | Cyanobacteria | CP007203.2 |
| 948 | GCA_000818015.1 | 1123016 | 74829 | Pseudomonas balearica DSM 6083 | DSM6083 (=SP1402) | Proteobacteria | CP007511.1 |
| 949 | GCA_001458455.1 | 75695 | 75695 | Chrysosporum ovalisporum | UAM-MAO | Cyanobacteria | CDHJ01000123.1 |
| 950 | GCA_000242075.2 | 883150 | 76832 | Myroides odoratimimus CCUG 10230 | CCUG 10230 | Bacteroidetes | KE161015.1 |
| 951 | GCA_000026985.1 | 644651 | 79967 | Erwinia pyrifoliae DSM 12163 | DSM 12163 | Proteobacteria | FN392235.1 |
| 952 | GCA_000027265.1 | 634499 | 79967 | Erwinia pyrifoliae Ep1/96 | Ep1/96 | Proteobacteria | FP236842.1 |
| 953 | GCA_000327045.1 | 765912 | 80679 | Thioflavicoccus mobilis 8321 | 8321 | Proteobacteria | CP003051.1 |
| 954 | GCA_001458195.1 | 81569 | 81569 | Ruegeria atlantica | CECT 4292 | Proteobacteria | CYPU01000011.1 |
| 955 | GCA_001586165.1 | 82983 | 82983 | Obesumbacterium proteus | DSM 2777 | Proteobacteria | CP014608.1 |
| 956 | GCA_000438825.1 | 1348660 | 82996 | Serratia plymuthica S13 | | Proteobacteria | CP006566.1 |
| 957 | GCA_000622345.1 | 1443112 | 83219 | Sulfitobacter mediterraneus KCTC 32188 | KCTC 32188 | Proteobacteria | JASH01000008.1 |
| 958 | GCA_001605015.1 | 83263 | 83263 | Aminobacter aminovorans | KCTC 2477 | Proteobacteria | CP015005.1 |
| 959 | GCA_000203935.1 | 94122 | 94122 | Shewanella sp. ANA-3 | ANA-3 | Proteobacteria | CP000469.1 |
| 960 | GCA_000067205.1 | 94624 | 94624 | Bordetella petrii | DSM 12804 | Proteobacteria | AM902716.1 |
| 961 | GCA_000333135.2 | 985077 | 95486 | Burkholderia cenocepacia BC7 | | Proteobacteria | ALIZ02000089.1 |
| 962 | GCA_000333155.2 | 985076 | 95486 | Burkholderia cenocepacia K56-2Valvano | K56-2Valvano | Proteobacteria | ALJA02000012.1 |
| 963 | GCA_000981305.1 | 95486 | 95486 | Burkholderia cenocepacia | K56-2 | Proteobacteria | LAUA01000017.1 |
| 964 | GCA_001523855.1 | 101571 | 101571 | Burkholderia ubonensis | ABCPW-28 | Proteobacteria | LOVU01000053.1 |
| 965 | GCA_001528985.1 | 101571 | 101571 | Burkholderia ubonensis | MSMB1162WGS | Proteobacteria | LPDS01000057.1 |
| 966 | GCA_001530375.1 | 101571 | 101571 | Burkholderia ubonensis | MSMB1473WGS | Proteobacteria | LPFJ01000144.1 |
| 967 | GCA_000317575.1 | 111780 | 102116 | Stanieria cyanosphaera PCC 7437 | PCC 7437 | Cyanobacteria | CP003653.1 |
| 968 | GCA_000025645.1 | 580331 | 108150 | Thermoanaerobacter italicus Ab9 | Ab9 | Firmicutes | CP001936.1 |
| 969 | GCA_000215105.1 | 743718 | 139208 | Isoptericola variabilis 225 | 225 | Actinobacteria | CP002810.1 |
| 970 | GCA_000024825.1 | 150340 | 150340 | Vibrio antiquarius | EX25 | Proteobacteria | CP001805.1 |
| 971 | GCA_000152485.1 | 150340 | 150340 | Vibrio antiquarius | EX25 | Proteobacteria | DS267811.1 |
| 972 | GCA_000759535.1 | 157783 | 157783 | Pseudomonas cremoricolorata | ND07 | Proteobacteria | CP009455.1 |
| 973 | GCA_000012985.1 | 349521 | 158327 | Hahella chejuensis KCTC 2396 | KCTC 2396 | Proteobacteria | CP000155.1 |
| 974 | GCA_001506165.1 | 158822 | 158822 | Cedecea neteri | ND02 | Proteobacteria | CP011427.1 |
| 975 | GCA_000181515.1 | 161528 | 161528 | Erythrobacter sp. SD-21 | SD-21 | Proteobacteria | ABCG01000002.1 |
| 976 | GCA_000069785.1 | 977880 | 164546 | Cupriavidus taiwanensis LMG 19424 | LMG19424 | Proteobacteria | CU633749.1 |
| 977 | GCA_000010305.1 | 379066 | 173480 | Gemmatimonas aurantiaca T-27 | T-27 | Gemmatimonadetes | AP009153.1 |
| 978 | GCA_000774145.1 | 1348058 | 183795 | Pseudomonas mediterranea CFBP 5447 | CFBP 5447 | Proteobacteria | KN322981.1 |
| 979 | GCA_000255295.1 | 1144275 | 184914 | Corallococcus coralloides DSM 2259 | DSM 2259 | Proteobacteria | CP003389.1 |
| 980 | GCA_000235975.2 | 1088721 | 205844 | Novosphingobium pentaromativorans US6-1 | US6-1 | Proteobacteria | AGFM01000037.1 |
| 981 | GCA_000767465.1 | 1088721 | 205844 | Novosphingobium pentaromativorans US6-1 | US6-1 | Proteobacteria | CP009291.1 |
| 982 | GCA_000091465.1 | 575788 | 212663 | Vibrio tasmaniensis LGP32 | LGP32 | Proteobacteria | FM954972.2 |
| 983 | GCA_000165815.1 | 215689 | 215689 | Erwinia sp. Ejp617 | Ejp617 | Proteobacteria | CP002124.1 |
| 984 | GCA_000015505.1 | 365044 | 216465 | Polaromonas naphthalenivorans CJ2 | CJ2 | Proteobacteria | CP000530.1 |
| 985 | GCA_000264315.1 | 1163408 | 219571 | Rhodanobacter fulvus Jip2 | Jip2 | Proteobacteria | AJXU01000037.1 |
| 986 | GCA_000315055.1 | 1129793 | 222814 | Paraglaciecola polaris LMG 21857 | LMG 21857 | Proteobacteria | BAER01000023.1 |
| 987 | GCA_000317495.1 | 1173022 | 241425 | Crinalium epipsammum PCC 9333 | PCC 9333 | Cyanobacteria | CP003620.1 |
| 988 | GCA_000742915.1 | 244366 | 244366 | Klebsiella variicola | BZ19 | Proteobacteria | JDWA01000007.1 |
| 989 | GCA_001048495.1 | 246167 | 246167 | Vibrio crassostreae | J5-19 | Proteobacteria | CCJU01000026.1 |
| 990 | GCA_001368875.1 | 246167 | 246167 | Vibrio crassostreae | J5-4 | Proteobacteria | CCJX01000059.1 |
| 991 | GCA_001580835.1 | 257438 | 257438 | Gluconobacter thailandicus | LMG 1741 | Proteobacteria | LHZM01000066.1 |
| 992 | GCA_000168275.1 | 262489 | 262489 | delta proteobacterium MLMS-1 | MLMS-1 | Proteobacteria | AAQF01000005.1 |
| 993 | GCA_001548015.1 | 270351 | 270351 | Methylobacterium aquaticum | MA-22A | Proteobacteria | AP014704.1 |
| 994 | GCA_000968535.1 | 1091494 | 271065 | Methylomicrobium alcaliphilum 20Z | 20Z | Proteobacteria | FO082060.1 |
| 995 | GCA_000825645.1 | 271420 | 271420 | Pseudomonas xanthomarina | S11 | Proteobacteria | CCYE01000007.1 |
| 996 | GCA_000280495.1 | 1177180 | 283686 | Kosakonia radicincitans DSM 16656 | DSM 16656 | Proteobacteria | JH725436.1 |
| 997 | GCA_000691205.1 | 1455607 | 283686 | Kosakonia radicincitans UMEnt01/12 | UMEnt01/12 | Proteobacteria | JDYJ01000004.1 |
| 998 | GCA_000317615.1 | 13035 | 292566 | Dactylococcopsis salina PCC 8305 | PCC 8305 | Cyanobacteria | CP003944.1 |
| 999 | GCA_000517385.1 | 1294265 | 308892 | Bacillus boroniphilus JCM 21738 | JCM 21738 | Firmicutes | BAUW01000042.1 |
| 1000 | GCA_000152965.1 | 314262 | 314262 | Roseobacter sp. MED193 | MED193 | Proteobacteria | CH902583.1 |
| 1001 | GCA_000171815.1 | 314289 | 314289 | Vibrio sp. AND4 | AND4 | Proteobacteria | ABGR01000004.1 |
| 1002 | GCA_001458175.1 | 321267 | 321267 | Shimia marina | CECT 7688 | Proteobacteria | CYPW01000010.1 |
| 1003 | GCA_001544495.1 | 326475 | 326475 | Paraburkholderia telluris | | Proteobacteria | FCNZ01000045.1 |
| 1004 | GCA_000013905.1 | 335284 | 330922 | Psychrobacter cryohalolentis K5 | K5 | Proteobacteria | CP000323.1 |
| 1005 | GCA_000382885.1 | 1301087 | 332056 | Sphingobium japonicum BiD32 | BiD32 | Proteobacteria | CAVK010000098.1 |
| 1006 | GCA_000015285.1 | 357804 | 357794 | Psychromonas ingrahamii 37 | 37 | Proteobacteria | CP000510.1 |
| 1007 | GCA_000302535.1 | 358220 | 358220 | Acidovorax sp. KKS102 | KKS102 | Proteobacteria | CP003872.1 |
| 1008 | GCA_000242115.2 | 1117958 | 359110 | Pseudomonas extremaustralis 14-3 substr. 14-3b | 14-3 substr. 14-3b | Proteobacteria | AHIP01000001.1 |
| 1009 | GCA_000145255.1 | 395494 | 370405 | Gallionella capsiferriformans ES-2 | ES-2 | Proteobacteria | CP002159.1 |
| 1010 | GCA_000241365.2 | 386428 | 386428 | Pseudoalteromonas sp. BSi20480 | BSi20480 | Proteobacteria | BADX01000166.1 |
| 1011 | GCA_001563285.1 | 407020 | 407020 | Sphingobium sp. MI1205 | MI1205 | Proteobacteria | CP005188.1 |
| 1012 | GCA_000166355.1 | 632292 | 413888 | Caldicellulosiruptor hydrothermalis 108 | 108 | Firmicutes | CP002219.1 |
| 1013 | GCA_000177355.1 | 679191 | 419005 | Prevotella amnii CRIS 21A-A | CRIS 21A-A | Bacteroidetes | ADFQ01000093.1 |
| 1014 | GCA_001553225.1 | 419005 | 419005 | Prevotella amnii | DNF00307 | Bacteroidetes | KQ960543.1 |
| 1015 | GCA_000421925.1 | 1346790 | 420233 | Sphingobium chinhatense IP26 | IP26 | Proteobacteria | AUDA01000024.1 |
| 1016 | GCA_001043885.1 | 1295136 | 427683 | Methylobacterium platani JCM 14648 | JCM 14648 | Proteobacteria | JXOD01000086.1 |
| 1017 | GCA_000633575.2 | 1450518 | 440518 | Sphingobium lucknowense F2 | F2 | Proteobacteria | JANF02000055.1 |
| 1018 | GCA_000023645.1 | 443144 | 443144 | Geobacter sp. M21 | M21 | Proteobacteria | CP001661.1 |
| 1019 | GCA_000157035.2 | 457388 | 457388 | Parabacteroides sp. 2_1_7 | 2_1_7 | Bacteroidetes | KQ236092.1 |
| 1020 | GCA_001517385.1 | 466044 | 466044 | Pannonibacter indicus | DSM 23407 | Proteobacteria | CYHE01000022.1 |
| 1021 | GCA_000143085.1 | 105559 | 473531 | Nitrosococcus watsonii C-113 | C-113 | Proteobacteria | CP002086.1 |
| 1022 | GCA_000943805.1 | 484429 | 484429 | Sphingobium sp. YBL2 | YBL2 | Proteobacteria | CP010954.1 |
| 1023 | GCA_000943805.1 | 484429 | 484429 | Sphingobium sp. YBL2 | YBL2 | Proteobacteria | CP010954.1 |
| 1024 | GCA_000242435.1 | 742743 | 487173 | Dialister succinatiphilus YIT 11850 | YIT 11850 | Firmicutes | JH591187.1 |
| 1025 | GCA_001527605.1 | 488732 | 488732 | Burkholderia diffusa | MSMB1544 | Proteobacteria | LOZQ01000052.1 |
| 1026 | GCA_000173035.1 | 495036 | 495036 | Geobacillus sp. G11MC16 | G11MC16 | Firmicutes | ABVH01000002.1 |
| 1027 | GCA_001545155.1 | 507626 | 507626 | Halomonas chromatireducens | AGD 8-3 | Proteobacteria | CP014226.1 |
| 1028 | GCA_000245735.2 | 1129374 | 545533 | Alishewanella jeotgali KCTC 22429 | KCTC 22429 | Proteobacteria | AHTH01000002.1 |
| 1029 | GCA_000225565.1 | 598467 | 598467 | Brenneria sp. EniD312 | EniD312 | Proteobacteria | CM001230.1 |
| 1030 | GCA_001499735.1 | 610332 | 610332 | Thiocapsa sp. KS1 | KS1 | Proteobacteria | LN848258.1 |
| 1031 | GCA_000188175.1 | 626939 | 626940 | Phascolarctobacterium succinatutens YIT 12067 | YIT 12067 | Firmicutes | GL830854.1 |
| 1032 | GCA_000307475.1 | 658661 | 658661 | Parabacteroides sp. D25 | D25 | Bacteroidetes | JH976502.1 |
| 1033 | GCA_001305515.1 | 665126 | 665126 | Prosthecomicrobium hirschii | 16 | Proteobacteria | LIYW01000001.1 |
| 1034 | GCA_001579845.1 | 674703 | 674703 | Rhodoplanes sp. Z2-YC6860 | YC6860 | Proteobacteria | CP007440.1 |
| 1035 | GCA_001077885.1 | 680026 | 680026 | Photobacterium swingsii | CAIM 1393 | Proteobacteria | LELC01000014.1 |
| 1036 | GCA_000253255.1 | 702113 | 702113 | Novosphingobium sp. PP1Y | PP1Y | Proteobacteria | FR856862.1 |
| 1037 | GCA_000253255.1 | 702113 | 702113 | Novosphingobium sp. PP1Y | PP1Y | Proteobacteria | FR856862.1 |
| 1038 | GCA_000024465.1 | 488538 | 767892 | Candidatus Puniceispirillum marinum IMCC1322 | IMCC1322 | Proteobacteria | CP001751.1 |
| 1039 | GCA_000422025.1 | 886559 | 886559 | Geobacillus sp. WSUCF1 | WSUCF1 | Firmicutes | ATCO01000102.1 |
| 1040 | GCA_001580035.1 | 936474 | 936474 | Sphingobium sp. 22B | 22B | Proteobacteria | LTAB01000001.1 |
| 1041 | GCA_000178975.2 | 1198114 | 940615 | Granulicella tundricola MP5ACTX9 | MP5ACTX9 | Acidobacteria | CP002480.1 |
| 1042 | GCA_000422205.1 | 941449 | 941449 | Desulfovibrio sp. X2 | X2 | Proteobacteria | ATHV01000015.1 |
| 1043 | GCA_000498695.1 | 977905 | 977905 | Bacillus sp. 17376 | 17376 | Firmicutes | KI547189.1 |
| 1044 | GCA_000801085.1 | 1076549 | 1076549 | Pantoea rodasii | ND03 | Proteobacteria | JTJJ01000016.1 |
| 1045 | GCA_000474655.1 | 1231725 | 1089444 | Dickeya solani D s0432-1 | D s0432-1 | Proteobacteria | AMWE01000004.1 |
| 1046 | GCA_000803195.1 | 1089444 | 1089444 | Dickeya solani | M005 | Proteobacteria | JSXD01000001.1 |
| 1047 | GCA_000972705.2 | 1637645 | 1118279 | Limnoraphis robusta CS-951 | CS-951 | Cyanobacteria | LATL02000068.1 |
| 1048 | GCA_001517305.1 | 1137284 | 1137284 | Marinomonas fungiae | JCM 18476 | Proteobacteria | CYHG01000005.1 |
| 1049 | GCA_000282175.1 | 1144323 | 1144323 | Pseudomonas sp. GM17 | GM17 | Proteobacteria | AKJU01000007.1 |
| 1050 | GCA_000282315.2 | 1144708 | 1144708 | Pseudomonas sp. GM41(2012) | GM41(2012) | Proteobacteria | AKJN02000007.1 |
| 1051 | GCA_000264335.1 | 1162282 | 1162282 | Rhodanobacter sp. 115 | 115 | Proteobacteria | AJXS01000176.1 |
| 1052 | GCA_000407765.1 | 1169321 | 1169321 | Escherichia sp. KTE114 | KTE114 | Proteobacteria | KE136626.1 |
| 1053 | GCA_000350705.1 | 1169331 | 1169331 | Escherichia sp. KTE11 | KTE11 | Proteobacteria | KB732246.1 |
| 1054 | GCA_000407925.1 | 1169349 | 1169349 | Escherichia sp. KTE31 | KTE31 | Proteobacteria | KE136722.1 |
| 1055 | GCA_001550165.1 | 1176302 | 1176302 | Sphingobium sp. AM | AM | Proteobacteria | LRUK01000005.1 |
| 1056 | GCA_000408325.1 | 1182659 | 1182659 | Escherichia sp. KTE52 | KTE52 | Proteobacteria | KE138680.1 |
| 1057 | GCA_000398905.1 | 1182695 | 1182695 | Klebsiella sp. KTE92 | KTE92 | Proteobacteria | KB976100.1 |
| 1058 | GCA_000408525.1 | 1182697 | 1182697 | Escherichia sp. KTE96 | KTE96 | Proteobacteria | KE137149.1 |
| 1059 | GCA_000408805.1 | 1182732 | 1182732 | Escherichia sp. KTE159 | KTE159 | Proteobacteria | KE137292.1 |
| 1060 | GCA_000316175.1 | 1207075 | 1207075 | Pseudomonas sp. UW4 | UW4 | Proteobacteria | CP003880.1 |
| 1061 | GCA_000801955.1 | 1217052 | 1217052 | Ralstonia sp. A12 | A12 | Proteobacteria | JSZO01000004.1 |
| 1062 | GCA_000369405.1 | 1217707 | 1217707 | Acinetobacter sp. ANC 3929 | ANC 3929 | Proteobacteria | KB849994.1 |
| 1063 | GCA_000342045.1 | 1244869 | 1244869 | Magnetospirillum caucaseum | SO-1 | Proteobacteria | AONQ01000075.1 |
| 1064 | GCA_000437815.1 | 1262687 | 1262687 | Acidaminococcus sp. CAG:542 | | Firmicutes | FR902027.1 |
| 1065 | GCA_000434475.1 | 1262870 | 1262870 | Dialister sp. CAG:486 | | Firmicutes | FR879630.1 |
| 1066 | GCA_000495795.1 | 1282360 | 1282360 | Asticcacaulis sp. AC460 | AC460 | Proteobacteria | AWGD01000018.1 |
| 1067 | GCA_000495835.1 | 1282361 | 1282361 | Asticcacaulis sp. AC402 | AC402 | Proteobacteria | AWGC01000023.1 |
| 1068 | GCA_000738435.1 | 666509 | 1284658 | Planktomarina temperata RCA23 | RCA23 | Proteobacteria | CP003984.1 |
| 1069 | GCA_001314555.1 | 1298881 | 1298881 | Idiomarinaceae bacterium HL-53 | | Proteobacteria | LIHO01000009.1 |
| 1070 | GCA_001458075.1 | 1298881 | 1298881 | Idiomarinaceae bacterium HL-53 | HL-53 | Proteobacteria | LN899469.1 |
| 1071 | GCA_000493055.1 | 1329826 | 1329826 | Enterobacter sp. MGH 22 | MGH 22 | Proteobacteria | KI535662.1 |
| 1072 | GCA_000416315.1 | 1330035 | 1330035 | Osedax symbiont Rs2 | | Proteobacteria | ASZI01000003.1 |
| 1073 | GCA_000445145.1 | 1114964 | 1332080 | Sphingobium baderi LL03 | LL03 | Proteobacteria | ATIB01000079.1 |
| 1074 | GCA_001046655.1 | 1114964 | 1332080 | Sphingobium baderi LL03 | LL03 | Proteobacteria | KQ130471.1 |
| 1075 | GCA_000445995.2 | 1345697 | 1345697 | Geobacillus sp. JF8 | JF8 | Firmicutes | CP006254.2 |
| 1076 | GCA_000788395.1 | 1348114 | 1348114 | Pseudoalteromonas sp. OCN003 | OCN003 | Proteobacteria | CP009888.1 |
| 1077 | GCA_001581925.1 | 1356306 | 1356306 | Ferrovum sp. PN-J185 | PN-J185 | Proteobacteria | LQZA01000004.1 |
| 1078 | GCA_001263175.1 | 1391653 | 1391653 | Vulgatibacter incomptus | DSM 27710 | Proteobacteria | CP012332.1 |
| 1079 | GCA_000475355.1 | 1396859 | 1396859 | Marinobacter sp. C1S70 | C1S70 | Proteobacteria | AXBW01000091.1 |
| 1080 | GCA_000475415.1 | 1397528 | 1397528 | Halomonas sp. PBN3 | PBN3 | Proteobacteria | AXCA01000185.1 |
| 1081 | GCA_001580565.1 | 1399969 | 1399969 | Burkholderia megalochromosomata | JC2949 | Proteobacteria | LRBF01000261.1 |
| 1082 | GCA_001028775.2 | 1406431 | 1406431 | Massilia sp. WF1 | WF1 | Proteobacteria | LELH02000007.1 |
| 1083 | GCA_000524665.1 | 1435973 | 1435973 | Alcanivorax sp. 97CO-5 | 97CO-5 | Proteobacteria | KI965903.1 |
| 1084 | GCA_000585055.1 | 1454003 | 1454003 | Candidatus Accumulibacter sp. BA-92 | | Proteobacteria | JEMX01000010.1 |
| 1085 | GCA_000648925.1 | 1458357 | 1458357 | Burkholderia jiangsuensis | MP-1 | Proteobacteria | JFHF01000072.1 |
| 1086 | GCA_000828915.1 | 1458426 | 1458426 | Comamonadaceae bacterium B1 | B1 | Proteobacteria | AP014569.1 |
| 1087 | GCA_001368815.1 | 1462524 | 1462524 | Paraliobacillus sp. PM-2 | PM-2 | Firmicutes | CTEI01000001.1 |
| 1088 | GCA_000828975.1 | 1469502 | 1469502 | Burkholderiales bacterium GJ-E10 | GJ-E10 | Proteobacteria | AP014683.1 |
| 1089 | GCA_000611145.2 | 1470558 | 1470558 | Cupriavidus sp. SK-3 | SK-3 | Proteobacteria | JFJV02000088.1 |
| 1090 | GCA_001402345.1 | 1481663 | 1481663 | Vibrio metoecus | YB5B06 | Proteobacteria | LBGQ01000030.1 |
| 1091 | GCA_001402675.1 | 1481663 | 1481663 | Vibrio metoecus | YB5B04 | Proteobacteria | LBGP01000018.1 |
| 1092 | GCA_000731675.1 | 1495066 | 1495066 | Pseudomonas sp. WCS358 | WCS358 | Proteobacteria | JMIT01000003.1 |
| 1093 | GCA_000732325.1 | 1499968 | 1499968 | Paenibacillus sp. TCA20 | TCA20 | Firmicutes | BBIW01000009.1 |
| 1094 | GCA_001529965.1 | 1503055 | 1503055 | Burkholderia territorii | MSMB1301WGS | Proteobacteria | LPEQ01000007.1 |
| 1095 | GCA_001530725.1 | 1503055 | 1503055 | Burkholderia territorii | MSMB1502WGS | Proteobacteria | LPFQ01000127.1 |
| 1096 | GCA_001530925.1 | 1503055 | 1503055 | Burkholderia territorii | MSMB1840WGS | Proteobacteria | LPGS01000070.1 |
| 1097 | GCA_000734975.2 | 1504981 | 1504981 | Halomonas sp. KO116 | KO116 | Proteobacteria | CP011052.1 |
| 1098 | GCA_000824845.1 | 1505933 | 1505933 | Mesorhizobium sp. ORS3359 | | Proteobacteria | CCNC01000071.1 |
| 1099 | GCA_000824805.1 | 1505946 | 1505946 | Mesorhizobium sp. SOD10 | | Proteobacteria | CCNA01000056.1 |
| 1100 | GCA_000802135.1 | 1531958 | 1531958 | Desulfosporosinus sp. BICA1-9 | | Firmicutes | JUEE01000226.1 |
| 1101 | GCA_000757485.1 | 1532557 | 1532557 | Achromobacter sp. RTa | RTa | Proteobacteria | JPYO01000042.1 |
| 1102 | GCA_000764205.1 | 1535422 | 1535422 | Thalassotalea sp. ND16A | ND16A | Proteobacteria | JQDZ01000100.1 |
| 1103 | GCA_001267535.1 | 1560201 | 1560201 | Erwinia iniecta | B120 | Proteobacteria | JRXE01000012.1 |
| 1104 | GCA_001267545.1 | 1560201 | 1560201 | Erwinia iniecta | B149 | Proteobacteria | JRXF01000008.1 |
| 1105 | GCA_000788125.1 | 1561068 | 1561068 | Acinetobacter sp. neg1 | neg1 | Proteobacteria | JSZD01000006.1 |
| 1106 | GCA_001411475.1 | 1563157 | 1563157 | Pseudomonas endophytica | BSTT44 | Proteobacteria | LLWH01000196.1 |
| 1107 | GCA_000934545.1 | 1565605 | 1565605 | Rhodocyclaceae bacterium PG1-Ca6 | PG1-Ca6 | Proteobacteria | CP010554.1 |
| 1108 | GCA_000831325.1 | 1572758 | 1572758 | Thauera sp. SWB20 | SWB20 | Proteobacteria | JTDM01000020.1 |
| 1109 | GCA_001455145.1 | 1307442 | 1583098 | Fusobacterium hwasookii ChDC F174 | ChDC F174 | Fusobacteria | CP013331.1 |
| 1110 | GCA_000836735.1 | 1588629 | 1588629 | Aeromonas sp. L_1B5_3 | L_1B5_3 | Proteobacteria | JXIR01000008.1 |
| 1111 | GCA_001439745.1 | 1615675 | 1615675 | Pseudomonas sp. DSM 28142 | DSM 28142 | Proteobacteria | JYLP01000005.1 |
| 1112 | GCA_001548095.1 | 1615909 | 1615909 | Geminocystis sp. NIES-3708 | NIES-3708 | Cyanobacteria | AP014815.1 |
| 1113 | GCA_000802035.2 | 1619235 | 1619235 | Pseudomonas sp. BICA1-14 | | Proteobacteria | JUEC02000002.1 |
| 1114 | GCA_000958445.1 | 1619246 | 1619246 | Enterobacter sp. 42202 | 42202 | Proteobacteria | JZYH01000003.1 |
| 1115 | GCA_000955795.1 | 1619951 | 1619951 | Burkholderiaceae bacterium 26 | 26 | Proteobacteria | JYOB01000010.1 |
| 1116 | GCA_000960545.1 | 1620393 | 1620393 | Vibrio sp. S457-15 | S457-15 | Proteobacteria | JYOQ01000119.1 |
| 1117 | GCA_000960425.1 | 1620394 | 1620394 | Vibrio sp. S512-13 | S512-13 | Proteobacteria | JYOR01000079.1 |
| 1118 | GCA_001011155.1 | 1620421 | 1620421 | Hoeflea sp. IMCC20628 | IMCC20628 | Proteobacteria | CP011479.1 |
| 1119 | GCA_001044505.1 | 1628278 | 1628278 | Nitrospina sp. SCGC_AAA799_A02 | | Nitrospinae | JZKI01000044.1 |
| 1120 | GCA_000961835.1 | 1629711 | 1629711 | Pseudomonas sp. BRH_c35 | | Proteobacteria | LADX01000225.1 |
| 1121 | GCA_000961805.1 | 1629714 | 1629714 | Peptococcaceae bacterium BRH_c23 | | Firmicutes | LADV01000103.1 |
| 1122 | GCA_001011715.1 | 1643812 | 1643812 | Enterobacter sp. GN02174 | GN02174 | Proteobacteria | LDCI01000011.1 |
| 1123 | GCA_001023195.1 | 1643814 | 1643814 | Enterobacter sp. GN02204 | GN02204 | Proteobacteria | LEEQ01000037.1 |
| 1124 | GCA_001011975.1 | 1643815 | 1643815 | Enterobacter sp. GN02225 | GN02225 | Proteobacteria | LDCL01000065.1 |
| 1125 | GCA_001077815.1 | 1648404 | 1648404 | Erythrobacter sp. s21-N3 | s21-N3 | Proteobacteria | CP011310.1 |
| 1126 | GCA_001023235.1 | 1659705 | 1659705 | Enterobacter sp. GN02275 | GN02275 | Proteobacteria | LEEK01000036.1 |
| 1127 | GCA_001022355.1 | 1659709 | 1659709 | Enterobacter sp. GN02366 | GN02366 | Proteobacteria | LEEC01000026.1 |
| 1128 | GCA_001022445.2 | 1659710 | 1659710 | Enterobacter sp. GN02454 | GN02454 | Proteobacteria | LEDW02000013.1 |
| 1129 | GCA_001022495.2 | 1659711 | 1659711 | Enterobacter sp. GN02457 | GN02457 | Proteobacteria | LEDV02000004.1 |
| 1130 | GCA_001022995.1 | 1659720 | 1659720 | Enterobacter sp. GN03191 | GN03191 | Proteobacteria | LECY01000030.1 |
| 1131 | GCA_001293045.1 | 1678678 | 1678678 | Burkholderia sp. HB1 | HB1 | Proteobacteria | CP012192.1 |
| 1132 | GCA_001412125.1 | 1699078 | 1699078 | Geobacillus sp. PA-3 | PA-3 | Firmicutes | LIDX01000059.1 |
| 1133 | GCA_001593285.1 | 1699622 | 1699622 | Psychrobacter sp. P2G3 | P2G3 | Proteobacteria | CP012529.1 |
| 1134 | GCA_001271345.1 | 1702325 | 1702325 | Chelatococcus sp. CO-6 | CO-6 | Proteobacteria | CP012398.1 |
| 1135 | GCA_001412595.1 | 1707785 | 1707785 | Massilia sp. WG5 | WG5 | Proteobacteria | CP012640.1 |
| 1136 | GCA_001458295.1 | 1715692 | 1715692 | Ruegeria sp. CECT 5091 | CECT 5091 | Proteobacteria | CYUD01000002.1 |
| 1137 | GCA_001458215.1 | 1715693 | 1715693 | Phaeobacter sp. CECT 7735 | CECT 7735 | Proteobacteria | CYTW01000001.1 |
| 1138 | GCA_001458695.1 | 1715989 | 1715989 | Candidatus Nitrospira inopinata | | Nitrospirae | LN885086.1 |
| 1139 | GCA_001529285.1 | 1729582 | 1729582 | Pseudomonas sp. TAA207 | TAA207 | Proteobacteria | LLWJ01000066.1 |
| 1140 | GCA_001529305.1 | 1729583 | 1729583 | Pseudomonas sp. TAD18 | TAD18 | Proteobacteria | LLWI01000076.1 |
| 1141 | GCA_001516045.1 | 1734396 | 1734396 | Desulfosporosinus sp. BRH_c37 | | Firmicutes | LOEW01000087.1 |
| 1142 | GCA_001421235.1 | 1735675 | 1735675 | Rhizobium sp. Leaf202 | Leaf202 | Proteobacteria | LMKD01000014.1 |
| 1143 | GCA_001421635.1 | 1735692 | 1735692 | Sphingomonas sp. Leaf25 | Leaf25 | Proteobacteria | LMKU01000030.1 |
| 1144 | GCA_001421985.1 | 1736231 | 1736231 | Rhizobium sp. Leaf68 | Leaf68 | Proteobacteria | LMLQ01000014.1 |
| 1145 | GCA_001423405.1 | 1736264 | 1736264 | Methylobacterium sp. Leaf123 | Leaf123 | Proteobacteria | LMNS01000004.1 |
| 1146 | GCA_001424265.1 | 1736280 | 1736280 | Acidovorax sp. Leaf160 | Leaf160 | Proteobacteria | LMOV01000023.1 |
| 1147 | GCA_001423245.1 | 1736336 | 1736336 | Aureimonas sp. Leaf324 | Leaf324 | Proteobacteria | LMOH01000012.1 |
| 1148 | GCA_001424045.1 | 1736355 | 1736355 | Rhizobium sp. Leaf371 | Leaf371 | Proteobacteria | LMQB01000026.1 |
| 1149 | GCA_001424985.1 | 1736429 | 1736429 | Rhizobium sp. Root1212 | Root1212 | Proteobacteria | LMDA01000022.1 |
| 1150 | GCA_001426805.1 | 1736462 | 1736462 | Brevundimonas sp. Root1423 | Root1423 | Proteobacteria | LMFL01000003.1 |
| 1151 | GCA_001429215.1 | 1736505 | 1736505 | Acidovorax sp. Root267 | Root267 | Proteobacteria | LMJC01000005.1 |
| 1152 | GCA_001428925.1 | 1736506 | 1736506 | Rhizobium sp. Root268 | Root268 | Proteobacteria | LMJD01000023.1 |
| 1153 | GCA_001424925.1 | 1736515 | 1736515 | Pseudomonas sp. Root329 | Root329 | Proteobacteria | LMCV01000007.1 |
| 1154 | GCA_001426345.1 | 1736537 | 1736537 | Devosia sp. Root436 | Root436 | Proteobacteria | LMEM01000015.1 |
| 1155 | GCA_001429505.1 | 1736544 | 1736544 | Bosea sp. Root483D1 | Root483D1 | Proteobacteria | LMJW01000003.1 |
| 1156 | GCA_001427465.1 | 1736566 | 1736566 | Pseudomonas sp. Root569 | Root569 | Proteobacteria | LMGQ01000003.1 |
| 1157 | GCA_001427825.1 | 1736569 | 1736569 | Brevundimonas sp. Root608 | Root608 | Proteobacteria | LMGT01000001.1 |
| 1158 | GCA_001429725.1 | 1736592 | 1736592 | Rhizobium sp. Root708 | Root708 | Proteobacteria | LMHV01000043.1 |
| 1159 | GCA_001414205.1 | 1737624 | 1737624 | Geobacillus sp. Sah69 | Sah69 | Firmicutes | LLKS01000066.1 |
| 1160 | GCA_001542405.1 | 1755240 | 1755240 | Rhizobium sp. BR10423 | BR10423 | Proteobacteria | LNCD01000088.1 |
| 1161 | GCA_001506075.1 | 1761910 | 1761910 | Vibrio sp. MEBiC08052 | MEBiC08052 | Proteobacteria | KQ947475.1 |
| 1162 | GCA_001469895.1 | 1766621 | 1766621 | Pseudoalteromonas sp. XI10 | XI10 | Proteobacteria | LOPY01000025.1 |
| 1163 | GCA_001544695.1 | 1777135 | 1777135 | Burkholderia sp. LMG 29317 | | Proteobacteria | FCOM01000073.1 |
| 1164 | GCA_900044055.1 | 1777138 | 1777138 | Burkholderia sp. LMG 29320 | | Proteobacteria | FCOX01000009.1 |
| 1165 | GCA_900044055.1 | 1777138 | 1777138 | Burkholderia sp. LMG 29320 | | Proteobacteria | FCOX01000002.1 |
| 1166 | GCA_900044055.1 | 1777138 | 1777138 | Burkholderia sp. LMG 29320 | | Proteobacteria | FCOX01000040.1 |
| 1167 | GCA_001544915.1 | 1777141 | 1777141 | Burkholderia sp. LMG 29323 | | Proteobacteria | FCOE01000003.1 |
| 1168 | GCA_001545035.1 | 1777143 | 1777143 | Burkholderia sp. LMG 29325 | | Proteobacteria | FCOJ01000099.1 |
| 1169 | GCA_001577625.1 | 1785949 | 1785949 | Halobacteroidaceae bacterium T328-2 | | Firmicutes | LSBO01000115.1 |
| 1170 | GCA_001581915.1 | 1789004 | 1789004 | Ferrovum sp. Z-31 | Z-31 | Proteobacteria | LRRD01000049.1 |
| 1171 | GCA_000286655.1 | 1216470 | 1812935 | Enterobacter sp. SST3 | SST3 | Proteobacteria | ALNS01000022.1 |
| 1172 | GCA_000467655.2 | 1334630 | 1812935 | Enterobacter cloacae EC_38VIM1 | EC_38VIM1 | Proteobacteria | ATHX01000012.1 |
| 1173 | GCA_000534155.1 | 1400152 | 1812935 | Enterobacter cloacae UCI 39 | UCI 39 | Proteobacteria | KI973119.1 |
| 1174 | GCA_001592395.1 | 1813182 | 1813182 | Geobacillus sp. JS12 | JS12 | Firmicutes | CP014749.1 |
| 1175 | GCA_000024525.1 | 504472 | 108 | Spirosoma linguale DSM 74 | DSM 74 | Bacteroidetes | CP001770.1 |
| 1176 | GCA_000253755.2 | 887281 | 195 | Campylobacter coli 7--1 | 7--1 | Proteobacteria | AIMZ01000005.1 |
| 1177 | GCA_000253935.2 | 887284 | 195 | Campylobacter coli 67-8 | 67-8 | Proteobacteria | AINI01000046.1 |
| 1178 | GCA_000254035.2 | 887304 | 195 | Campylobacter coli LMG 23341 | LMG 23341 | Proteobacteria | AINN01000049.1 |
| 1179 | GCA_000254055.2 | 887305 | 195 | Campylobacter coli LMG 23342 | LMG 23342 | Proteobacteria | AIN001000051.1 |
| 1180 | GCA_001416745.1 | 195 | 195 | Campylobacter coli | CVM N42205 | Proteobacteria | LBEO01000037.1 |
| 1181 | GCA_001418825.1 | 1440776 | 195 | Campylobacter coli CVM 41904 | CVM 41904 | Proteobacteria | JAJL01000029.1 |
| 1182 | GCA_001419395.1 | 1440016 | 195 | Campylobacter coli CVM 41963 | CVM 41963 | Proteobacteria | JAJR01000050.1 |
| 1183 | GCA_001441815.1 | 195 | 195 | Campylobacter coli | CVM N3506 | Proteobacteria | JOUR01000007.1 |
| 1184 | GCA_001442405.1 | 195 | 195 | Campylobacter coli | CVM N14840 | Proteobacteria | JOVV01000015.1 |
| 1185 | GCA_000705275.1 | 91352 | 198 | Campylobacter hyointestinalis subsp. hyointestinalis | DSM 19053 | Proteobacteria | JHQP01000002.1 |
| 1186 | GCA_000701165.2 | 1395570 | 292 | Burkholderia cepacia JBK9 | JBK9 | Proteobacteria | CP013730.1 |
| 1187 | GCA_001543265.1 | 296 | 296 | Pseudomonas fragi | P121 | Proteobacteria | CP013861.1 |
| 1188 | GCA_900048915.1 | 446 | 446 | Legionella pneumophila | 2531STDY5467317 | Proteobacteria | FJAM01000003.1 |
| 1189 | GCA_900051685.1 | 446 | 446 | Legionella pneumophila | 2531STDY5467385 | Proteobacteria | FJCH01000002.1 |
| 1190 | GCA_900052085.1 | 446 | 446 | Legionella pneumophila | 2531STDY5467299 | Proteobacteria | FJAW01000009.1 |
| 1191 | GCA_900059955.1 | 446 | 446 | Legionella pneumophila | 2531STDY5467397 | Proteobacteria | FJDI01000005.1 |
| 1192 | GCA_900060365.1 | 446 | 446 | Legionella pneumophila | 2531STDY5467373 | Proteobacteria | FJDE01000004.1 |
| 1193 | GCA_900061515.1 | 446 | 446 | Legionella pneumophila | 2531STDY5467344 | Proteobacteria | FJCG01000004.1 |
| 1194 | GCA_900061575.1 | 446 | 446 | Legionella pneumophila | 2531STDY5467389 | Proteobacteria | FJCV01000005.1 |
| 1195 | GCA_900070165.1 | 446 | 446 | Legionella pneumophila | 2531STDY5467373 | Proteobacteria | FJNL01000004.1 |
| 1196 | GCA_000176095.1 | 638315 | 450 | Legionella longbeachae D-4968 | D-4968 | Proteobacteria | ACZG01000001.1 |
| 1197 | GCA_000092265.1 | 1236608 | 480 | Moraxella catarrhalis BBH18 | BBH18 | Proteobacteria | CP002005.1 |
| 1198 | GCA_000192905.2 | 857581 | 480 | Moraxella catarrhalis 7169 | 7169 | Proteobacteria | AERC01000010.1 |
| 1199 | GCA_000192925.2 | 857579 | 480 | Moraxella catarrhalis 103P14B1 | 103P14B1 | Proteobacteria | AERE01000083.1 |
| 1200 | GCA_000192945.2 | 857578 | 480 | Moraxella catarrhalis 46P47B1 | 46P47B1 | Proteobacteria | AERF01000047.1 |
| 1201 | GCA_000192965.2 | 857577 | 480 | Moraxella catarrhalis 12P80B1 | 12P80B1 | Proteobacteria | AERG01000021.1 |
| 1202 | GCA_000192985.2 | 857576 | 480 | Moraxella catarrhalis BC1 | BC1 | Proteobacteria | AERH01000031.1 |
| 1203 | GCA_000193005.2 | 857575 | 480 | Moraxella catarrhalis BC7 | BC7 | Proteobacteria | AERI01000026.1 |
| 1204 | GCA_000193045.2 | 857573 | 480 | Moraxella catarrhalis CO72 | CO72 | Proteobacteria | AERK01000003.1 |
| 1205 | GCA_000193065.2 | 857572 | 480 | Moraxella catarrhalis 101P30B1 | 101P30B1 | Proteobacteria | AEPC01000015.1 |
| 1206 | GCA_000302495.1 | 749219 | 480 | Moraxella catarrhalis RH4 | RH4 | Proteobacteria | AMSO01000008.1 |
| 1207 | GCA_000766665.1 | 480 | 480 | Moraxella catarrhalis | 25239 | Proteobacteria | CP007669.1 |
| 1208 | GCA_001281005.1 | 546 | 546 | Citrobacter freundii | P10159 | Proteobacteria | CP012554.1 |
| 1209 | GCA_000952375.1 | 550 | 550 | Enterobacter cloacae | MNCRE12 | Proteobacteria | JYME01000006.1 |
| 1210 | GCA_001005095.1 | 553 | 553 | Pantoea ananatis | MR5 | Proteobacteria | LBFU01000095.1 |
| 1211 | GCA_000738105.1 | 180957 | 554 | Pectobacterium carotovorum subsp. brasiliense | CFIA1009 | Proteobacteria | JPSN01000016.1 |
| 1212 | GCA_000808235.1 | 180957 | 554 | Pectobacterium carotovorum subsp. brasiliense | YCD21 | Proteobacteria | JUJJ01000006.1 |
| 1213 | GCA_000006665.1 | 155864 | 562 | Escherichia coli O157:H7 str. EDL933 | EDL933 | Proteobacteria | AE005174.2 |
| 1214 | GCA_000008865.1 | 386585 | 562 | Escherichia coli O157:H7 str. Sakai | Sakai substr. RIMD 0509952 | Proteobacteria | BA000007.2 |
| 1215 | GCA_000019385.1 | 481805 | 562 | Escherichia coli ATCC 8739 | ATCC 8739 | Proteobacteria | CP000946.1 |
| 1216 | GCA_000019645.1 | 439855 | 562 | Escherichia coli SMS-3-5 | SMS-3-5 | Proteobacteria | CP000970.1 |
| 1217 | GCA_000021125.1 | 444450 | 562 | Escherichia coli O157:H7 str. EC4115 | EC4115 | Proteobacteria | CP001164.1 |
| 1218 | GCA_000022225.1 | 544404 | 562 | Escherichia coli O157:H7 str. TW14359 | TW14359 | Proteobacteria | CP001368.1 |
| 1219 | GCA_000025165.1 | 701177 | 562 | Escherichia coli O55:H7 str. CB9615 | CB9615 | Proteobacteria | CP001846.1 |
| 1220 | GCA_000155125.1 | 502346 | 562 | Escherichia coli O157:H7 str. TW14588 | TW14588 | Proteobacteria | CM000662.1 |
| 1221 | GCA_000164335.1 | 749547 | 562 | Escherichia coli MS 187-1 | MS 187-1 | Proteobacteria | GG772497.1 |
| 1222 | GCA_000171915.1 | 444451 | 562 | Escherichia coli O157:H7 str. EC4196 | EC4196 | Proteobacteria | ABHO01000006.1 |
| 1223 | GCA_000171935.1 | 444452 | 562 | Escherichia coli O157:H7 str. EC4113 | EC4113 | Proteobacteria | ABHP01000015.1 |
| 1224 | GCA_000171955.1 | 444453 | 562 | Escherichia coli O157:H7 str. EC4076 | EC4076 | Proteobacteria | ABHQ01000017.1 |
| 1225 | GCA_000171975.1 | 478004 | 562 | Escherichia coli O157:H7 str. EC4401 | EC4401 | Proteobacteria | ABHR01000005.1 |
| 1226 | GCA_000171995.1 | 478005 | 562 | Escherichia coli O157:H7 str. EC4486 | EC4486 | Proteobacteria | ABHS01000002.1 |
| 1227 | GCA_000172015.1 | 478006 | 562 | Escherichia coli O157:H7 str. EC4501 | EC4501 | Proteobacteria | ABHT01000004.1 |
| 1228 | GCA_000172035.1 | 478008 | 562 | Escherichia coli O157:H7 str. EC869 | EC869 | Proteobacteria | ABHU01000001.1 |
| 1229 | GCA_000172055.1 | 478007 | 562 | Escherichia coli O157:H7 str. EC508 | EC508 | Proteobacteria | ABHW01000003.1 |
| 1230 | GCA_000176615.2 | 656437 | 562 | Escherichia coli TA143 | TA143 | Proteobacteria | GL884236.1 |
| 1231 | GCA_000181735.1 | 444447 | 562 | Escherichia coli O157:H7 str. EC4206 | EC4206 | Proteobacteria | ABHK02000001.1 |
| 1232 | GCA_000181755.1 | 444448 | 562 | Escherichia coli O157:H7 str. EC4045 | EC4045 | Proteobacteria | ABHL02000001.1 |
| 1233 | GCA_000181775.1 | 444449 | 562 | Escherichia coli O157:H7 str. EC4042 | EC4042 | Proteobacteria | ABHM02000001.1 |
| 1234 | GCA_000187285.4 | 926026 | 562 | Escherichia coli O157:H7 str. G5101 | G5101 | Proteobacteria | AETX01000114.1 |
| 1235 | GCA_000187305.2 | 926027 | 562 | Escherichia coli O157:H-str. 493-89 | 493-89 | Proteobacteria | AETY01000104.1 |
| 1236 | GCA_000187325.2 | 926028 | 562 | Escherichia coli O157:H-str. H 2687 | H 2687 | Proteobacteria | AETZ01000110.1 |
| 1237 | GCA_000187365.2 | 926030 | 562 | Escherichia coli O55:H7 str. USDA 5905 | USDA 5905 | Proteobacteria | AEUB01000059.1 |
| 1238 | GCA_000187385.2 | 926032 | 562 | Escherichia coli O157:H7 str. LSU-61 | LSU-61 | Proteobacteria | AEUC01000091.1 |
| 1239 | GCA_000192665.2 | 997824 | 562 | Escherichia coli O157:H7 str. 1044 | 1044 | Proteobacteria | AERP01000018.1 |
| 1240 | GCA_000192685.2 | 997825 | 562 | Escherichia coli O157:H7 str. 1125 | 1125 | Proteobacteria | AERR01000012.1 |
| 1241 | GCA_000193955.2 | 941435 | 562 | Escherichia coli O157:H7 str. EC1212 | EC1212 | Proteobacteria | AERQ01000011.1 |
| 1242 | GCA_000245515.1 | 1048689 | 562 | Escherichia coli O55:H7 str. RM12579 | RM12579 | Proteobacteria | CP003109.1 |
| 1243 | GCA_000249375.2 | 868149 | 562 | Escherichia coli DEC4A | DEC4A | Proteobacteria | AIFK01000052.1 |
| 1244 | GCA_000249395.2 | 868150 | 562 | Escherichia coli DEC4B | DEC4B | Proteobacteria | AIFL01000076.1 |
| 1245 | GCA_000249495.2 | 868155 | 562 | Escherichia coli DEC5A | DEC5A | Proteobacteria | AIFQ01000037.1 |
| 1246 | GCA_000249535.2 | 868157 | 562 | Escherichia coli DEC5C | DEC5C | Proteobacteria | AIFS01000052.1 |
| 1247 | GCA_000249555.2 | 868158 | 562 | Escherichia coli DEC5D | DEC5D | Proteobacteria | AIFT01000001.1 |
| 1248 | GCA_000261145.1 | 656421 | 562 | Escherichia coli M919 | M919 | Proteobacteria | JH659569.1 |
| 1249 | GCA_000262125.1 | 741093 | 562 | Escherichia coli Xuzhou21 | Xuzhou21 | Proteobacteria | CP001925.1 |
| 1250 | GCA_000267005.2 | 1005471 | 562 | Escherichia coli FDA505 | FDA505 | Proteobacteria | JH692596.1 |
| 1251 | GCA_000267025.1 | 1005535 | 562 | Escherichia coli EC1738 | EC1738 | Proteobacteria | AKMN01000003.1 |
| 1252 | GCA_000267045.1 | 1005536 | 562 | Escherichia coli EC1734 | EC1734 | Proteobacteria | AKMO01000001.1 |
| 1253 | GCA_000267065.1 | 1005520 | 562 | Escherichia coli TW09109 | TW09109 | Proteobacteria | AKLY01000005.1 |
| 1254 | GCA_000267085.1 | 1005497 | 562 | Escherichia coli PA22 | PA22 | Proteobacteria | AKLI01000001.1 |
| 1255 | GCA_000267105.1 | 1005508 | 562 | Escherichia coli PA40 | PA40 | Proteobacteria | AKLQ01000001.1 |
| 1256 | GCA_000267125.1 | 1005515 | 562 | Escherichia coli TW06591 | TW06591 | Proteobacteria | AKLT01000044.1 |
| 1257 | GCA_000267145.1 | 1005517 | 562 | Escherichia coli TW10246 | TW10246 | Proteobacteria | AKLV01000003.1 |
| 1258 | GCA_000267165.1 | 1005518 | 562 | Escherichia coli TW11039 | TW11039 | Proteobacteria | AKLW01000006.1 |
| 1259 | GCA_000267185.1 | 1005522 | 562 | Escherichia coli TW10119 | TW10119 | Proteobacteria | AKMA01000001.1 |
| 1260 | GCA_000267205.2 | 1005548 | 562 | Escherichia coli EC1863 | EC1863 | Proteobacteria | JH692972.1 |
| 1261 | GCA_000267225.2 | 1005540 | 562 | Escherichia coli EC1845 | EC1845 | Proteobacteria | JH693448.1 |
| 1262 | GCA_000267245.2 | 1005534 | 562 | Escherichia coli EC4448 | EC4448 | Proteobacteria | JH693935.1 |
| 1263 | GCA_000267265.2 | 1005533 | 562 | Escherichia coli EC4437 | EC4437 | Proteobacteria | JH694340.1 |
| 1264 | GCA_000267285.2 | 1005532 | 562 | Escherichia coli EC4436 | EC4436 | Proteobacteria | JH694746.1 |
| 1265 | GCA_000267305.2 | 1005531 | 562 | Escherichia coli EC4439 | EC4439 | Proteobacteria | JH697080.1 |
| 1266 | GCA_000267325.1 | 1005530 | 562 | Escherichia coli EC4402 | EC4402 | Proteobacteria | JH697727.1 |
| 1267 | GCA_000267345.2 | 1005529 | 562 | Escherichia coli EC4013 | EC4013 | Proteobacteria | JH698305.1 |
| 1268 | GCA_000267365.2 | 1005528 | 562 | Escherichia coli EC4422 | EC4422 | Proteobacteria | JH695870.1 |
| 1269 | GCA_000267385.2 | 1005527 | 562 | Escherichia coli EC4421 | EC4421 | Proteobacteria | JH696600.1 |
| 1270 | GCA_000267405.2 | 1005526 | 562 | Escherichia coli TW14301 | TW14301 | Proteobacteria | JH695863.1 |
| 1271 | GCA_000267425.2 | 1005525 | 562 | Escherichia coli TW14313 | TW14313 | Proteobacteria | JH695399.1 |
| 1272 | GCA_000267445.2 | 1005524 | 562 | Escherichia coli EC4196 | EC4196 | Proteobacteria | JH699193.1 |
| 1273 | GCA_000267465.2 | 1005523 | 562 | Escherichia coli EC4203 | EC4203 | Proteobacteria | JH699677.1 |
| 1274 | GCA_000267485.2 | 1005473 | 562 | Escherichia coli FDA517 | FDA517 | Proteobacteria | JH701994.1 |
| 1275 | GCA_000267505.2 | 1005475 | 562 | Escherichia coli FRIK1996 | FRIK1996 | Proteobacteria | JH698283.1 |
| 1276 | GCA_000267525.2 | 1005476 | 562 | Escherichia coli FRIK1985 | FRIK1985 | Proteobacteria | JH701532.1 |
| 1277 | GCA_000267585.2 | 1005521 | 562 | Escherichia coli TW09195 | TW09195 | Proteobacteria | JH700736.1 |
| 1278 | GCA_000267605.2 | 1005519 | 562 | Escherichia coli TW09098 | TW09098 | Proteobacteria | JH702452.1 |
| 1279 | GCA_000267625.2 | 1005516 | 562 | Escherichia coli TW07945 | TW07945 | Proteobacteria | JH702467.1 |
| 1280 | GCA_000267645.2 | 1005510 | 562 | Escherichia coli PA42 | PA42 | Proteobacteria | JH702836.1 |
| 1281 | GCA_000267665.2 | 1005509 | 562 | Escherichia coli PA41 | PA41 | Proteobacteria | JH703744.1 |
| 1282 | GCA_000267685.2 | 1005507 | 562 | Escherichia coli PA39 | PA39 | Proteobacteria | JH704563.1 |
| 1283 | GCA_000267705.2 | 1005504 | 562 | Escherichia coli PA33 | PA33 | Proteobacteria | JH705112.1 |
| 1284 | GCA_000267725.2 | 1005503 | 562 | Escherichia coli PA32 | PA32 | Proteobacteria | JH705678.1 |
| 1285 | GCA_000267745.2 | 1005502 | 562 | Escherichia coli PA31 | PA31 | Proteobacteria | JH707077.1 |
| 1286 | GCA_000267765.2 | 1005501 | 562 | Escherichia coli PA28 | PA28 | Proteobacteria | JH705685.1 |
| 1287 | GCA_000267785.2 | 1005500 | 562 | Escherichia coli PA25 | PA25 | Proteobacteria | JH706538.1 |
| 1288 | GCA_000267805.2 | 1005499 | 562 | Escherichia coli PA24 | PA24 | Proteobacteria | JH707528.1 |
| 1289 | GCA_000267825.2 | 1005496 | 562 | Escherichia coli PA15 | PA15 | Proteobacteria | JH707559.1 |
| 1290 | GCA_000267845.2 | 1005495 | 562 | Escherichia coli PA14 | PA14 | Proteobacteria | JH708600.1 |
| 1291 | GCA_000267865.2 | 1005494 | 562 | Escherichia coli PA10 | PA10 | Proteobacteria | JH709066.1 |
| 1292 | GCA_000267885.2 | 1005493 | 562 | Escherichia coli PA9 | PA9 | Proteobacteria | JH709102.1 |
| 1293 | GCA_000267905.2 | 1005490 | 562 | Escherichia coli PA5 | PA5 | Proteobacteria | JH709975.1 |
| 1294 | GCA_000267925.2 | 1005488 | 562 | Escherichia coli PA3 | PA3 | Proteobacteria | JH711390.1 |
| 1295 | GCA_000267945.2 | 1005481 | 562 | Escherichia coli 93-001 | 93-001 | Proteobacteria | JH710997.1 |
| 1296 | GCA_000267965.2 | 1005478 | 562 | Escherichia coli FRIK1990 | FRIK1990 | Proteobacteria | JH710503.1 |
| 1297 | GCA_000302715.1 | 1005483 | 562 | Escherichia coli FRIK920 | FRIK920 | Proteobacteria | AMTP01000126.1 |
| 1298 | GCA_000302735.1 | 1005491 | 562 | Escherichia coli PA7 | PA7 | Proteobacteria | AMTQ01000008.1 |
| 1299 | GCA_000303235.2 | 1005441 | 562 | Escherichia coli 0.1304 | 0.1304 | Proteobacteria | JH943332.1 |
| 1300 | GCA_000303275.2 | 1005482 | 562 | Escherichia coli FRIK523 | FRIK523 | Proteobacteria | JH943936.1 |
| 1301 | GCA_000303295.2 | 1005486 | 562 | Escherichia coli NE098 | NE098 | Proteobacteria | JH946265.1 |
| 1302 | GCA_000303315.2 | 1005554 | 562 | Escherichia coli EC1870 | EC1870 | Proteobacteria | JH945214.1 |
| 1303 | GCA_000303335.2 | 1005553 | 562 | Escherichia coli EC1869 | EC1869 | Proteobacteria | JH946714.1 |
| 1304 | GCA_000303355.2 | 1005552 | 562 | Escherichia coli EC1868 | EC1868 | Proteobacteria | JH944350.1 |
| 1305 | GCA_000303375.2 | 1005551 | 562 | Escherichia coli EC1866 | EC1866 | Proteobacteria | JH944315.1 |
| 1306 | GCA_000303415.2 | 1005549 | 562 | Escherichia coli EC1864 | EC1864 | Proteobacteria | JH945870.1 |
| 1307 | GCA_000303435.2 | 1005547 | 562 | Escherichia coli EC1862 | EC1862 | Proteobacteria | JH952466.1 |
| 1308 | GCA_000303455.2 | 1005546 | 562 | Escherichia coli EC1856 | EC1856 | Proteobacteria | JH952880.1 |
| 1309 | GCA_000303475.2 | 1005545 | 562 | Escherichia coli EC1850 | EC1850 | Proteobacteria | JH952886.1 |
| 1310 | GCA_000303495.2 | 1005544 | 562 | Escherichia coli EC1849 | EC1849 | Proteobacteria | JH953696.1 |
| 1311 | GCA_000303515.2 | 1005543 | 562 | Escherichia coli EC1848 | EC1848 | Proteobacteria | JH955442.1 |
| 1312 | GCA_000303535.2 | 1005542 | 562 | Escherichia coli EC1847 | EC1847 | Proteobacteria | JH963080.1 |
| 1313 | GCA_000303555.2 | 1005541 | 562 | Escherichia coli EC1846 | EC1846 | Proteobacteria | JH956315.1 |
| 1314 | GCA_000303575.2 | 1005539 | 562 | Escherichia coli EC1737 | EC1737 | Proteobacteria | JH963529.1 |
| 1315 | GCA_000303595.2 | 1005538 | 562 | Escherichia coli EC1736 | EC1736 | Proteobacteria | JH965238.1 |
| 1316 | GCA_000303615.2 | 1005537 | 562 | Escherichia coli EC1735 | EC1735 | Proteobacteria | JH958312.1 |
| 1317 | GCA_000303675.2 | 1005506 | 562 | Escherichia coli PA38 | PA38 | Proteobacteria | JH966117.1 |
| 1318 | GCA_000303695.2 | 1005505 | 562 | Escherichia coli PA34 | PA34 | Proteobacteria | JH963946.1 |
| 1319 | GCA_000303715.2 | 1005474 | 562 | Escherichia coli FDA506 | FDA506 | Proteobacteria | JH964536.1 |
| 1320 | GCA_000303735.2 | 1005472 | 562 | Escherichia coli FDA507 | FDA507 | Proteobacteria | JH963945.1 |
| 1321 | GCA_000303755.2 | 1005470 | 562 | Escherichia coli FDA504 | FDA504 | Proteobacteria | JH961871.1 |
| 1322 | GCA_000303775.2 | 1005477 | 562 | Escherichia coli FRIK1999 | FRIK1999 | Proteobacteria | JH962623.1 |
| 1323 | GCA_000303795.2 | 1005480 | 562 | Escherichia coli FRIK1997 | FRIK1997 | Proteobacteria | JH961362.1 |
| 1324 | GCA_000303815.2 | 1005485 | 562 | Escherichia coli NE1487 | NE1487 | Proteobacteria | JH957512.1 |
| 1325 | GCA_000303835.2 | 1005484 | 562 | Escherichia coli NE037 | NE037 | Proteobacteria | JH960043.1 |
| 1326 | GCA_000303855.2 | 1005479 | 562 | Escherichia coli FRIK2001 | FRIK2001 | Proteobacteria | JH960978.1 |
| 1327 | GCA_000303875.2 | 1005489 | 562 | Escherichia coli PA4 | PA4 | Proteobacteria | JH960549.1 |
| 1328 | GCA_000303895.2 | 1005498 | 562 | Escherichia coli PA23 | PA23 | Proteobacteria | JH959990.1 |
| 1329 | GCA_000303915.2 | 1005514 | 562 | Escherichia coli PA49 | PA49 | Proteobacteria | JH959133.1 |
| 1330 | GCA_000303935.2 | 1005511 | 562 | Escherichia coli PA45 | PA45 | Proteobacteria | JH958691.1 |
| 1331 | GCA_000303955.2 | 1005555 | 562 | Escherichia coli TT12B | TT12B | Proteobacteria | JH957216.1 |
| 1332 | GCA_000303975.2 | 1005557 | 562 | Escherichia coli MA6 | MA6 | Proteobacteria | JH956821.1 |
| 1333 | GCA_000303995.2 | 1005558 | 562 | Escherichia coli 5905 | 5905 | Proteobacteria | JH958373.1 |
| 1334 | GCA_000304015.2 | 1005559 | 562 | Escherichia coli CB7326 | CB7326 | Proteobacteria | JH956766.1 |
| 1335 | GCA_000304035.2 | 1005560 | 562 | Escherichia coli EC96038 | EC96038 | Proteobacteria | JH955436.1 |
| 1336 | GCA_000304055.2 | 1005561 | 562 | Escherichia coli 5412 | 5412 | Proteobacteria | JH954949.1 |
| 1337 | GCA_000304815.2 | 1005450 | 562 | Escherichia coli 3.4870 | 3.4870 | Proteobacteria | JH970627.1 |
| 1338 | GCA_000304835.2 | 1005463 | 562 | Escherichia coli 8.0416 | 8.0416 | Proteobacteria | AMTN01000133.1 |
| 1339 | GCA_000304855.2 | 1005469 | 562 | Escherichia coli 10.0821 | 10.0821 | Proteobacteria | AMTO01000004.1 |
| 1340 | GCA_000304875.2 | 1005452 | 562 | Escherichia coli 5.2239 | 5.2239 | Proteobacteria | JH970990.1 |
| 1341 | GCA_000305155.2 | 1005455 | 562 | Escherichia coli 6.0172 | 6.0172 | Proteobacteria | JH970109.1 |
| 1342 | GCA_000305375.2 | 1005461 | 562 | Escherichia coli 8.0586 | 8.0586 | Proteobacteria | JH969648.1 |
| 1343 | GCA_000305395.2 | 1005460 | 562 | Escherichia coli 8.2524 | 8.2524 | Proteobacteria | JH969044.1 |
| 1344 | GCA_000305415.2 | 1005466 | 562 | Escherichia coli 10.0833 | 10.0833 | Proteobacteria | JH969098.1 |
| 1345 | GCA_000305435.2 | 1005467 | 562 | Escherichia coli 10.0869 | 10.0869 | Proteobacteria | JH968727.1 |
| 1346 | GCA_000305455.2 | 1005397 | 562 | Escherichia coli 88.0221 | 88.0221 | Proteobacteria | JH968005.1 |
| 1347 | GCA_000316325.2 | 1005396 | 562 | Escherichia coli 88.1467 | 88.1467 | Proteobacteria | KB000019.1 |
| 1348 | GCA_000316345.2 | 1005398 | 562 | Escherichia coli 88.1042 | 88.1042 | Proteobacteria | KB006081.1 |
| 1349 | GCA_000316365.2 | 1005399 | 562 | Escherichia coli 89.0511 | 89.0511 | Proteobacteria | KB007266.1 |
| 1350 | GCA_000316385.2 | 1005400 | 562 | Escherichia coli 90.0091 | 90.0091 | Proteobacteria | KB007294.1 |
| 1351 | GCA_000316405.2 | 1005401 | 562 | Escherichia coli 90.0039 | 90.0039 | Proteobacteria | KB006791.1 |
| 1352 | GCA_000316425.2 | 1005402 | 562 | Escherichia coli 90.2281 | 90.2281 | Proteobacteria | KB005715.1 |
| 1353 | GCA_000316445.2 | 1005403 | 562 | Escherichia coli 93.0055 | 93.0055 | Proteobacteria | KB004131.1 |
| 1354 | GCA_000316465.1 | 1005404 | 562 | Escherichia coli 93.0056 | 93.0056 | Proteobacteria | KB004997.1 |
| 1355 | GCA_000316485.1 | 1005405 | 562 | Escherichia coli 94.0618 | 94.0618 | Proteobacteria | KB005707.1 |
| 1356 | GCA_000316505.2 | 1005420 | 562 | Escherichia coli 97.0007 | 97.0007 | Proteobacteria | ANMC01000275.1 |
| 1357 | GCA_000316545.2 | 1005422 | 562 | Escherichia coli 99.0678 | 99.0678 | Proteobacteria | ANME01000013.1 |
| 1358 | GCA_000316565.2 | 1005424 | 562 | Escherichia coli 99.0713 | 99.0713 | Proteobacteria | ANMF01000001.1 |
| 1359 | GCA_000316705.2 | 1005421 | 562 | Escherichia coli 99.0672 | 99.0672 | Proteobacteria | KB004968.1 |
| 1360 | GCA_000316725.2 | 1005418 | 562 | Escherichia coli 97.1742 | 97.1742 | Proteobacteria | KB004341.1 |
| 1361 | GCA_000316745.2 | 1005416 | 562 | Escherichia coli 97.0003 | 97.0003 | Proteobacteria | JH999738.1 |
| 1362 | GCA_000316765.2 | 1005415 | 562 | Escherichia coli 96.0107 | 96.0107 | Proteobacteria | KB003449.1 |
| 1363 | GCA_000316785.2 | 1005412 | 562 | Escherichia coli 96.0939 | 96.0939 | Proteobacteria | KB003084.1 |
| 1364 | GCA_000316805.2 | 1005413 | 562 | Escherichia coli 96.0932 | 96.0932 | Proteobacteria | KB003092.1 |
| 1365 | GCA_000316825.2 | 1005411 | 562 | Escherichia coli 96.0427 | 96.0427 | Proteobacteria | KB002687.1 |
| 1366 | GCA_000316845.2 | 1005406 | 562 | Escherichia coli 95.0183 | 95.0183 | Proteobacteria | KB002150.1 |
| 1367 | GCA_000316865.2 | 1005407 | 562 | Escherichia coli 95.1288 | 95.1288 | Proteobacteria | KB001319.1 |
| 1368 | GCA_000316885.2 | 1005408 | 562 | Escherichia coli 95.0943 | 95.0943 | Proteobacteria | KB000789.1 |
| 1369 | GCA_000316905.2 | 1005410 | 562 | Escherichia coli 96.0428 | 96.0428 | Proteobacteria | KB000504.1 |
| 1370 | GCA_000318425.2 | 1005414 | 562 | Escherichia coli 96.0109 | 96.0109 | Proteobacteria | KB009454.1 |
| 1371 | GCA_000318445.2 | 1005419 | 562 | Escherichia coli 97.0010 | 97.0010 | Proteobacteria | KB008877.1 |
| 1372 | GCA_000326965.1 | 1169382 | 562 | Escherichia coli KTE125 | KTE125 | Proteobacteria | KB732191.1 |
| 1373 | GCA_000334975.2 | 1005433 | 562 | Escherichia coli 99.0815 | 99.0815 | Proteobacteria | AODZ01000274.1 |
| 1374 | GCA_000334995.2 | 1005430 | 562 | Escherichia coli 99.0816 | 99.0816 | Proteobacteria | AOEA01000217.1 |
| 1375 | GCA_000335015.2 | 1005437 | 562 | Escherichia coli 99.1793 | 99.1793 | Proteobacteria | AOEF01000014.1 |
| 1376 | GCA_000335035.2 | 1005436 | 562 | Escherichia coli 99.1805 | 99.1805 | Proteobacteria | AOEG01000281.1 |
| 1377 | GCA_000335055.2 | 1051353 | 562 | Escherichia coli ATCC 700728 | ATCC 700728 | Proteobacteria | AOEH01000126.1 |
| 1378 | GCA_000335075.2 | 1005512 | 562 | Escherichia coli PA47 | PA47 | Proteobacteria | AOEM01000157.1 |
| 1379 | GCA_000335095.2 | 1005513 | 562 | Escherichia coli PA48 | PA48 | Proteobacteria | AOEN01000122.1 |
| 1380 | GCA_000335115.1 | 1051348 | 562 | Escherichia coli 7.1982 | 7.1982 | Proteobacteria | AOEP01000011.1 |
| 1381 | GCA_000335135.1 | 1005439 | 562 | Escherichia coli 99.1781 | 99.1781 | Proteobacteria | AOEQ01000012.1 |
| 1382 | GCA_000335155.2 | 1005435 | 562 | Escherichia coli 99.1762 | 99.1762 | Proteobacteria | AOER01000006.1 |
| 1383 | GCA_000335175.2 | 1051352 | 562 | Escherichia coli PA35 | PA35 | Proteobacteria | AOES01000151.1 |
| 1384 | GCA_000335195.2 | 1051347 | 562 | Escherichia coli 3.4880 | 3.4880 | Proteobacteria | AOET01000122.1 |
| 1385 | GCA_000335215.2 | 1005423 | 562 | Escherichia coli 99.0670 | 99.0670 | Proteobacteria | AOEV01000013.1 |
| 1386 | GCA_000335235.2 | 1005434 | 562 | Escherichia coli 99.0814 | 99.0814 | Proteobacteria | AODY01000209.1 |
| 1387 | GCA_000335255.2 | 1005432 | 562 | Escherichia coli 99.0839 | 99.0839 | Proteobacteria | AOEB01000180.1 |
| 1388 | GCA_000335275.2 | 1005431 | 562 | Escherichia coli 99.0848 | 99.0848 | Proteobacteria | AOEC01000227.1 |
| 1389 | GCA_000335295.2 | 1005440 | 562 | Escherichia coli 99.1753 | 99.1753 | Proteobacteria | AOED01000247.1 |
| 1390 | GCA_000335315.2 | 1005438 | 562 | Escherichia coli 99.1775 | 99.1775 | Proteobacteria | AOEE01000251.1 |
| 1391 | GCA_000335335.2 | 1051351 | 562 | Escherichia coli PA19 | PA19 | Proteobacteria | AOEJ01000133.1 |
| 1392 | GCA_000335355.2 | 1005487 | 562 | Escherichia coli PA2 | PA2 | Proteobacteria | AOEL01000002.1 |
| 1393 | GCA_000335415.2 | 1051349 | 562 | Escherichia coli PA11 | PA11 | Proteobacteria | AOEI01000002.1 |
| 1394 | GCA_000335435.2 | 1051350 | 562 | Escherichia coli PA13 | PA13 | Proteobacteria | AOEK01000001.1 |
| 1395 | GCA_000335455.2 | 1005492 | 562 | Escherichia coli PA8 | PA8 | Proteobacteria | AOEO01000008.1 |
| 1396 | GCA_000340455.2 | 1081887 | 562 | Escherichia coli 09BKT078844 | 09BKT078844 | Proteobacteria | KB453231.1 |
| 1397 | GCA_000350025.2 | 1090930 | 562 | Escherichia coli ONT:H33 str. C48/93 | C48/93 | Proteobacteria | AHAX01000074.1 |
| 1398 | GCA_000350045.2 | 1090929 | 562 | Escherichia coli O127:H27 str. C43/90 | C43/90 | Proteobacteria | AHAW01000071.1 |
| 1399 | GCA_000351625.1 | 1182673 | 562 | Escherichia coli KTE66 | KTE66 | Proteobacteria | KB732762.1 |
| 1400 | GCA_000351985.1 | 1182733 | 562 | Escherichia coli KTE161 | KTE161 | Proteobacteria | KB732934.1 |
| 1401 | GCA_000352325.1 | 1182657 | 562 | Escherichia coli KTE50 | KTE50 | Proteobacteria | KB733431.1 |
| 1402 | GCA_000352965.1 | 1181748 | 562 | Escherichia coli KTE202 | KTE202 | Proteobacteria | KB733186.1 |
| 1403 | GCA_000357685.2 | 1125651 | 562 | Escherichia coli P0304799.3 | P0304799.3 | Proteobacteria | APZX01000029.1 |
| 1404 | GCA_000456685.1 | 1280993 | 562 | Escherichia coli HVH 43 (4-2173468) | HVH 43 (4-2173468) | Proteobacteria | KE699249.1 |
| 1405 | GCA_000456705.1 | 1280994 | 562 | Escherichia coli HVH 44 (4-2298570) | HVH 44 (4-2298570) | Proteobacteria | KE699258.1 |
| 1406 | GCA_000458705.1 | 1281109 | 562 | Escherichia coli HVH 182 (4-0985554) | HVH 182 (4-0985554) | Proteobacteria | KE700608.1 |
| 1407 | GCA_000458915.1 | 1281120 | 562 | Escherichia coli HVH 193 (4-3331423) | HVH 193 (4-3331423) | Proteobacteria | KE700785.1 |
| 1408 | GCA_000460315.1 | 1281191 | 562 | Escherichia coli UMEA 3144-1 | UMEA 3144-1 | Proteobacteria | KE701670.1 |
| 1409 | GCA_000460355.1 | 1281194 | 562 | Escherichia coli UMEA 3151-1 | UMEA 3151-1 | Proteobacteria | KE701689.1 |
| 1410 | GCA_000461915.2 | 1151226 | 562 | Escherichia coli B26-2 | B26-2 | Proteobacteria | AVQV01000001.1 |
| 1411 | GCA_000461935.2 | 1081889 | 562 | Escherichia coli 09BKT076207 | 09BKT076207 | Proteobacteria | AVQR01000067.1 |
| 1412 | GCA_000461955.2 | 1151213 | 562 | Escherichia coli B102 | B102 | Proteobacteria | AVQS01000008.1 |
| 1413 | GCA_000461975.2 | 1151219 | 562 | Escherichia coli B29-1 | B29-1 | Proteobacteria | AVQY01000001.1 |
| 1414 | GCA_000461995.2 | 1151214 | 562 | Escherichia coli B107 | B107 | Proteobacteria | AVQT01000005.1 |
| 1415 | GCA_000462025.2 | 1151225 | 562 | Escherichia coli B26-1 | B26-1 | Proteobacteria | AVQU01000001.1 |
| 1416 | GCA_000462045.2 | 1151217 | 562 | Escherichia coli B28-1 | B28-1 | Proteobacteria | AVQW01000010.1 |
| 1417 | GCA_000462065.2 | 1151222 | 562 | Escherichia coli B36-2 | B36-2 | Proteobacteria | AVRB01000001.1 |
| 1418 | GCA_000462085.2 | 1151215 | 562 | Escherichia coli B7-1 | B7-1 | Proteobacteria | AVRC01000049.1 |
| 1419 | GCA_000462105.2 | 1151220 | 562 | Escherichia coli B29-2 | B29-2 | Proteobacteria | AVQZ01000120.1 |
| 1420 | GCA_000462125.2 | 1151221 | 562 | Escherichia coli B36-1 | B36-1 | Proteobacteria | AVRA01000107.1 |
| 1421 | GCA_000462145.2 | 1081891 | 562 | Escherichia coli T1282_01 | T1282_01 | Proteobacteria | AVRM01000132.1 |
| 1422 | GCA_000462165.2 | 1081893 | 562 | Escherichia coli T924_01 | T924_01 | Proteobacteria | AVRP01000002.1 |
| 1423 | GCA_000462185.2 | 1151237 | 562 | Escherichia coli B15 | B15 | Proteobacteria | AVSB01000112.1 |
| 1424 | GCA_000462225.2 | 1207035 | 562 | Escherichia coli 14A | 14A | Proteobacteria | AVRQ01000005.1 |
| 1425 | GCA_000462245.2 | 1207038 | 562 | Escherichia coli 2886-75 | 2886-75 | Proteobacteria | AVRR01000001.1 |
| 1426 | GCA_000462265.2 | 1081896 | 562 | Escherichia coli T1840_97 | T1840_97 | Proteobacteria | AVRN01000121.1 |
| 1427 | GCA_000462305.2 | 1081888 | 562 | Escherichia coli 08BKT055439 | 08BKT055439 | Proteobacteria | AVRI01000134.1 |
| 1428 | GCA_000462325.2 | 1081895 | 562 | Escherichia coli 08BKT77219 | 08BKT77219 | Proteobacteria | AVRJ01000125.1 |
| 1429 | GCA_000462345.2 | 1081894 | 562 | Escherichia coli 09BKT024447 | 09BKT024447 | Proteobacteria | AVRK01000010.1 |
| 1430 | GCA_000462365.2 | 1081890 | 562 | Escherichia coli Bd5610_99 | Bd5610_99 | Proteobacteria | AVRL01000137.1 |
| 1431 | GCA_000462385.2 | 1081892 | 562 | Escherichia coli T234_00 | T234_00 | Proteobacteria | AVRO01000124.1 |
| 1432 | GCA_000462405.2 | 1151216 | 562 | Escherichia coli B7-2 | B7-2 | Proteobacteria | AVRD01000050.1 |
| 1433 | GCA_000462425.2 | 1151231 | 562 | Escherichia coli B93 | B93 | Proteobacteria | AVRE01000046.1 |
| 1434 | GCA_000462445.2 | 1151232 | 562 | Escherichia coli B94 | B94 | Proteobacteria | AVRF01000052.1 |
| 1435 | GCA_000462465.2 | 1151233 | 562 | Escherichia coli B95 | B95 | Proteobacteria | AVRG01000053.1 |
| 1436 | GCA_000462485.2 | 1151234 | 562 | Escherichia coli B103 | B103 | Proteobacteria | AVRS01000001.1 |
| 1437 | GCA_000462505.2 | 1151243 | 562 | Escherichia coli B104 | B104 | Proteobacteria | AVRT01000005.1 |
| 1438 | GCA_000462525.2 | 1151247 | 562 | Escherichia coli B105 | B105 | Proteobacteria | AVRU01000005.1 |
| 1439 | GCA_000462545.2 | 1151248 | 562 | Escherichia coli B106 | B106 | Proteobacteria | AVRV01000001.1 |
| 1440 | GCA_000462565.2 | 550673 | 562 | Escherichia coli B108 | B108 | Proteobacteria | AVRW01000007.1 |
| 1441 | GCA_000462585.2 | 1151244 | 562 | Escherichia coli B109 | B109 | Proteobacteria | AVRX01000001.1 |
| 1442 | GCA_000462605.2 | 1151249 | 562 | Escherichia coli B112 | B112 | Proteobacteria | AVRY01000010.1 |
| 1443 | GCA_000462625.2 | 1151250 | 562 | Escherichia coli B113 | B113 | Proteobacteria | AVRZ01000133.1 |
| 1444 | GCA_000462645.2 | 1151251 | 562 | Escherichia coli B114 | B114 | Proteobacteria | AVSA01000001.1 |
| 1445 | GCA_000462665.2 | 1151238 | 562 | Escherichia coli B17 | B17 | Proteobacteria | AVSC01000121.1 |
| 1446 | GCA_000462685.2 | 1151227 | 562 | Escherichia coli B40-1 | B40-1 | Proteobacteria | AVSD01000010.1 |
| 1447 | GCA_000462705.2 | 1151228 | 562 | Escherichia coli B40-2 | B40-2 | Proteobacteria | AVSE01000003.1 |
| 1448 | GCA_000462725.2 | 1151246 | 562 | Escherichia coli B49-2 | B49-2 | Proteobacteria | AVSF01000006.1 |
| 1449 | GCA_000462745.2 | 1151224 | 562 | Escherichia coli B5-2 | B5-2 | Proteobacteria | AVSG01000001.1 |
| 1450 | GCA_000462765.2 | 1151239 | 562 | Escherichia coli B83 | B83 | Proteobacteria | AVSH01000001.1 |
| 1451 | GCA_000462785.2 | 1151240 | 562 | Escherichia coli B84 | B84 | Proteobacteria | AVSI01000012.1 |
| 1452 | GCA_000462805.2 | 1151241 | 562 | Escherichia coli B85 | B85 | Proteobacteria | AVSJ01000007.1 |
| 1453 | GCA_000462825.2 | 1151242 | 562 | Escherichia coli B86 | B86 | Proteobacteria | AVSK01000136.1 |
| 1454 | GCA_000462845.2 | 1151235 | 562 | Escherichia coli B89 | B89 | Proteobacteria | AVSL01000001.1 |
| 1455 | GCA_000462865.2 | 1151236 | 562 | Escherichia coli B90 | B90 | Proteobacteria | AVSM01000129.1 |
| 1456 | GCA_000462885.2 | 1207037 | 562 | Escherichia coli Tx1686 | Tx1686 | Proteobacteria | AVSN01000001.1 |
| 1457 | GCA_000462905.2 | 1207036 | 562 | Escherichia coli Tx3800 | Tx3800 | Proteobacteria | AVSO01000014.1 |
| 1458 | GCA_000473725.2 | 1389955 | 562 | Escherichia coli ATCC 35150 | ATCC 35150 | Proteobacteria | AWXM02000046.1 |
| 1459 | GCA_000477495.2 | 1151218 | 562 | Escherichia coli B28-2 | B28-2 | Proteobacteria | AVQX01000012.1 |
| 1460 | GCA_000488355.1 | 1268982 | 562 | Escherichia coli 907672 | 907672 | Proteobacteria | KI531258.1 |
| 1461 | GCA_000492235.1 | 1328450 | 562 | Escherichia coli BIDMC 39 | BIDMC 39 | Proteobacteria | KI535383.1 |
| 1462 | GCA_000494995.1 | 1281069 | 562 | Escherichia coli HVH 136 (4-5970458) | HVH 136 (4-5970458) | Proteobacteria | KI538714.1 |
| 1463 | GCA_000496345.2 | 1404261 | 562 | Escherichia coli ATCC BAA-2192 | ATCC BAA-2192 | Proteobacteria | AYGW02000190.1 |
| 1464 | GCA_000498255.1 | 1097579 | 562 | Escherichia coli P4-96 | P4-96 | Proteobacteria | AHHO01000034.1 |
| 1465 | GCA_000498275.1 | 1117111 | 562 | Escherichia coli P4-NR | P4-NR | Proteobacteria | AHHP01000046.1 |
| 1466 | GCA_000520035.1 | 1248902 | 562 | Escherichia coli O145:H28 str. RM13514 | RM13514 | Proteobacteria | CP006027.1 |
| 1467 | GCA_000601155.1 | 1444132 | 562 | Escherichia coli 1-110-08_S3_C1 | 1-110-08_S3_C1 | Proteobacteria | JHDN01000003.1 |
| 1468 | GCA_000601175.1 | 1444160 | 562 | Escherichia coli 1-110-08_S3_C2 | 1-110-08_S3_C2 | Proteobacteria | JHDM01000091.1 |
| 1469 | GCA_000601335.1 | 1444190 | 562 | Escherichia coli 1-110-08_S3_C3 | 1-110-08_S3_C3 | Proteobacteria | JHDL01000001.1 |
| 1470 | GCA_000614085.2 | 1446647 | 562 | Escherichia coli O157:H7 str. 2011EL-2288 | 2011EL-2288 | Proteobacteria | JHKE01000007.1 |
| 1471 | GCA_000614255.1 | 1446644 | 562 | Escherichia coli O157:H7 str. 2011EL-2114 | 2011EL-2114 | Proteobacteria | JHKH01000002.1 |
| 1472 | GCA_000614305.2 | 1446645 | 562 | Escherichia coli O157:H7 str. 2011EL-2286 | 2011EL-2286 | Proteobacteria | JHKG01000013.1 |
| 1473 | GCA_000614345.2 | 1446650 | 562 | Escherichia coli O157:H7 str. 2011EL-2312 | 2011EL-2312 | Proteobacteria | JHKB01000003.1 |
| 1474 | GCA_000614405.2 | 1446646 | 562 | Escherichia coli O157:H7 str. 2011EL-2287 | 2011EL-2287 | Proteobacteria | JHKF01000001.1 |
| 1475 | GCA_000614645.2 | 1446643 | 562 | Escherichia coli O157:H7 str. 2011EL-2113 | 2011EL-2113 | Proteobacteria | JHKI01000015.1 |
| 1476 | GCA_000614685.2 | 1446648 | 562 | Escherichia coli O157:H7 str. 2011EL-2289 | 2011EL-2289 | Proteobacteria | JHKD01000001.1 |
| 1477 | GCA_000614805.2 | 1446656 | 562 | Escherichia coli O157:H7 str. F7350 | F7350 | Proteobacteria | JHJN01000036.1 |
| 1478 | GCA_000614885.2 | 1446622 | 562 | Escherichia coli O157:H7 str. 2010C-4979C1 | 2010C-4979C1 | Proteobacteria | JHMK01000017.1 |
| 1479 | GCA_000614965.1 | 1446619 | 562 | Escherichia coli O157:H7 str. 2009EL1705 | 2009EL1705 | Proteobacteria | JHGE01000007.1 |
| 1480 | GCA_000615015.1 | 1446621 | 562 | Escherichia coli O157:H7 str. 2009EL2109 | 2009EL2109 | Proteobacteria | JHGC01000001.1 |
| 1481 | GCA_000615085.1 | 1446587 | 562 | Escherichia coli O145:NM str. 2010C-3507 | 2010C-3507 | Proteobacteria | JHFW01000006.1 |
| 1482 | GCA_000615145.1 | 1446589 | 562 | Escherichia coli O145:NM str. 2010C-3509 | 2010C-3509 | Proteobacteria | JHFU01000107.1 |
| 1483 | GCA_000615175.1 | 1446590 | 562 | Escherichia coli O145:NM str. 2010C-3510 | 2010C-3510 | Proteobacteria | JHFT01000001.1 |
| 1484 | GCA_000615225.2 | 1446591 | 562 | Escherichia coli O145:NM str. 2010C-3511 | 2010C-3511 | Proteobacteria | JHFS01000035.1 |
| 1485 | GCA_000615265.1 | 1446592 | 562 | Escherichia coli O145:NM str. 2010C-3516 | 2010C-3516 | Proteobacteria | JHFR01000094.1 |
| 1486 | GCA_000615305.1 | 1446593 | 562 | Escherichia coli O145:NM str. 2010C-3517 | 2010C-3517 | Proteobacteria | JHFQ01000117.1 |
| 1487 | GCA_000615345.1 | 1446594 | 562 | Escherichia coli O145:NM str. 2010C-3518 | 2010C-3518 | Proteobacteria | JHFP01000105.1 |
| 1488 | GCA_000615385.1 | 1446595 | 562 | Escherichia coli O145:NM str. 2010C-3521 | 2010C-3521 | Proteobacteria | JHFO01000022.1 |
| 1489 | GCA_000615415.2 | 1446596 | 562 | Escherichia coli O145:NM str. 2010C-3526 | 2010C-3526 | Proteobacteria | JHFN01000018.1 |
| 1490 | GCA_000615695.1 | 1446633 | 562 | Escherichia coli O157:H7 str. 2011EL-2101 | 2011EL-2101 | Proteobacteria | JHKS01000001.1 |
| 1491 | GCA_000615745.2 | 1446634 | 562 | Escherichia coli O157:H7 str. 2011EL-2103 | 2011EL-2103 | Proteobacteria | JHKR01000031.1 |
| 1492 | GCA_000615785.2 | 1446635 | 562 | Escherichia coli O157:H7 str. 2011EL-2104 | 2011EL-2104 | Proteobacteria | JHKQ01000016.1 |
| 1493 | GCA_000615815.2 | 1446636 | 562 | Escherichia coli O157:H7 str. 2011EL-2105 | 2011EL-2105 | Proteobacteria | JHKP01000014.1 |
| 1494 | GCA_000615865.2 | 1446637 | 562 | Escherichia coli O157:H7 str. 2011EL-2106 | 2011EL-2106 | Proteobacteria | JHKO01000009.1 |
| 1495 | GCA_000615925.2 | 1446638 | 562 | Escherichia coli O157:H7 str. 2011EL-2107 | 2011EL-2107 | Proteobacteria | JHKN01000006.1 |
| 1496 | GCA_000615965.2 | 1446639 | 562 | Escherichia coli O157:H7 str. 2011EL-2108 | 2011EL-2108 | Proteobacteria | JHKM01000031.1 |
| 1497 | GCA_000616005.2 | 1446640 | 562 | Escherichia coli O157:H7 str. 2011EL-2109 | 2011EL-2109 | Proteobacteria | JHKL01000015.1 |
| 1498 | GCA_000616035.2 | 1446641 | 562 | Escherichia coli O157:H7 str. 2011EL-2111 | 2011EL-2111 | Proteobacteria | JHKK01000003.1 |
| 1499 | GCA_000616075.2 | 1446642 | 562 | Escherichia coli O157:H7 str. 2011EL-2112 | 2011EL-2112 | Proteobacteria | JHKJ01000005.1 |
| 1500 | GCA_000616365.1 | 1446623 | 562 | Escherichia coli O157:H7 str. 2011EL-1107 | 2011EL-1107 | Proteobacteria | JHLK01000001.1 |
| 1501 | GCA_000616405.2 | 1446624 | 562 | Escherichia coli O157:H7 str. 2011EL-2090 | 2011EL-2090 | Proteobacteria | JHLI01000033.1 |
| 1502 | GCA_000616425.2 | 1446625 | 562 | Escherichia coli O157:H7 str. 2011EL-2091 | 2011EL-2091 | Proteobacteria | JHLH01000026.1 |
| 1503 | GCA_000616445.2 | 1446626 | 562 | Escherichia coli O157:H7 str. 2011EL-2092 | 2011EL-2092 | Proteobacteria | JHLG01000030.1 |
| 1504 | GCA_000616465.2 | 1446627 | 562 | Escherichia coli O157:H7 str. 2011EL-2093 | 2011EL-2093 | Proteobacteria | JHLF01000012.1 |
| 1505 | GCA_000616485.2 | 1446628 | 562 | Escherichia coli O157:H7 str. 2011EL-2094 | 2011EL-2094 | Proteobacteria | JHLE01000001.1 |
| 1506 | GCA_000616505.2 | 1446629 | 562 | Escherichia coli O157:H7 str. 2011EL-2096 | 2011EL-2096 | Proteobacteria | JHLD01000033.1 |
| 1507 | GCA_000616525.2 | 1446630 | 562 | Escherichia coli O157:H7 str. 2011EL-2097 | 2011EL-2097 | Proteobacteria | JHLC01000005.1 |
| 1508 | GCA_000616545.2 | 1446631 | 562 | Escherichia coli O157:H7 str. 2011EL-2098 | 2011EL-2098 | Proteobacteria | JHLB01000003.1 |
| 1509 | GCA_000616585.2 | 1446613 | 562 | Escherichia coli O157:H7 str. 08-3037 | 08-3037 | Proteobacteria | JHKZ01000011.1 |
| 1510 | GCA_000616605.2 | 1446614 | 562 | Escherichia coli O157:H7 str. 08-3527 | 08-3527 | Proteobacteria | JHKY01000032.1 |
| 1511 | GCA_000616645.2 | 1446615 | 562 | Escherichia coli O157:H7 str. 08-4169 | 08-4169 | Proteobacteria | JHKW01000004.1 |
| 1512 | GCA_000616665.2 | 1446586 | 562 | Escherichia coli O145:NM str. 08-4270 | 08-4270 | Proteobacteria | JHKV01000030.1 |
| 1513 | GCA_000616705.2 | 1446632 | 562 | Escherichia coli O157:H7 str. 2011EL-2099 | 2011EL-2099 | Proteobacteria | JHKT01000015.1 |
| 1514 | GCA_000616745.1 | 1446692 | 562 | Escherichia coli O157:H7 str. K5806 | K5806 | Proteobacteria | JHHZ01000001.1 |
| 1515 | GCA_000617345.1 | 1446585 | 562 | Escherichia coli O145:NM str. 06-3484 | 06-3484 | Proteobacteria | JHNN01000002.1 |
| 1516 | GCA_000617385.1 | 1446745 | 562 | Escherichia coli O55:H7 str. 06-3555 | 06-3555 | Proteobacteria | JHNL01000009.1 |
| 1517 | GCA_000617445.2 | 1446609 | 562 | Escherichia coli O157:H7 str. 06-3745 | 06-3745 | Proteobacteria | JHNI01000020.1 |
| 1518 | GCA_000617665.2 | 1446611 | 562 | Escherichia coli O157:H7 str. 07-3091 | 07-3091 | Proteobacteria | JHNF01000007.1 |
| 1519 | GCA_000617705.2 | 1446610 | 562 | Escherichia coli O157:H7 str. 06-4039 | 06-4039 | Proteobacteria | JHNG01000005.1 |
| 1520 | GCA_000617725.2 | 1446612 | 562 | Escherichia coli O157:H7 str. 07-3391 | 07-3391 | Proteobacteria | JHNE01000001.1 |
| 1521 | GCA_000617765.2 | 1446597 | 562 | Escherichia coli O145:NM str. 2010C-4557C2 | 2010C-4557C2 | Proteobacteria | JHNA01000067.1 |
| 1522 | GCA_000617965.1 | 1446680 | 562 | Escherichia coli O157:H7 str. K4405 | K4405 | Proteobacteria | JHIN01000100.1 |
| 1523 | GCA_000618025.1 | 1446663 | 562 | Escherichia coli O157:H7 str. K1420 | K1420 | Proteobacteria | JHJF01000008.1 |
| 1524 | GCA_000618045.1 | 1446653 | 562 | Escherichia coli O157:H7 str. F6749 | F6749 | Proteobacteria | JHJQ01000001.1 |
| 1525 | GCA_000618065.2 | 1446658 | 562 | Escherichia coli O157:H7 str. F7384 | F7384 | Proteobacteria | JHJL01000002.1 |
| 1526 | GCA_000618105.2 | 1446652 | 562 | Escherichia coli O157:H7 str. F6142 | F6142 | Proteobacteria | JHJT01000009.1 |
| 1527 | GCA_000618185.1 | 1446660 | 562 | Escherichia coli O157:H7 str. G5303 | G5303 | Proteobacteria | JHJI01000027.1 |
| 1528 | GCA_000618225.2 | 1446657 | 562 | Escherichia coli O157:H7 str. F7377 | F7377 | Proteobacteria | JHJM01000002.1 |
| 1529 | GCA_000618245.1 | 1446661 | 562 | Escherichia coli O157:H7 str. H2495 | H2495 | Proteobacteria | JHJH01000020.1 |
| 1530 | GCA_000618265.2 | 1446655 | 562 | Escherichia coli O157:H7 str. F6751 | F6751 | Proteobacteria | JHJO01000004.1 |
| 1531 | GCA_000618285.2 | 1446662 | 562 | Escherichia coli O157:H7 str. H2498 | H2498 | Proteobacteria | JHJG01000003.1 |
| 1532 | GCA_000618305.2 | 1446654 | 562 | Escherichia coli O157:H7 str. F6750 | F6750 | Proteobacteria | JHJP01000010.1 |
| 1533 | GCA_000618325.1 | 1446667 | 562 | Escherichia coli O157:H7 str. K1796 | K1796 | Proteobacteria | JHJA01000003.1 |
| 1534 | GCA_000618345.1 | 1446668 | 562 | Escherichia coli O157:H7 str. K1845 | K1845 | Proteobacteria | JHIZ01000038.1 |
| 1535 | GCA_000618385.1 | 1446666 | 562 | Escherichia coli O157:H7 str. K1795 | K1795 | Proteobacteria | JHJB01000012.1 |
| 1536 | GCA_000618445.1 | 1446669 | 562 | Escherichia coli O157:H7 str. K1921 | K1921 | Proteobacteria | JHIY01000012.1 |
| 1537 | GCA_000618465.1 | 1446670 | 562 | Escherichia coli O157:H7 str. K1927 | K1927 | Proteobacteria | JHIX01000108.1 |
| 1538 | GCA_000618485.1 | 1446671 | 562 | Escherichia coli O157:H7 str. K2188 | K2188 | Proteobacteria | JHIW01000103.1 |
| 1539 | GCA_000618505.1 | 1446672 | 562 | Escherichia coli O157:H7 str. K2191 | K2191 | Proteobacteria | JHIV01000063.1 |
| 1540 | GCA_000618525.1 | 1446675 | 562 | Escherichia coli O157:H7 str. K2581 | K2581 | Proteobacteria | JHIS01000013.1 |
| 1541 | GCA_000618545.1 | 1446676 | 562 | Escherichia coli O157:H7 str. K2622 | K2622 | Proteobacteria | JHIR01000010.1 |
| 1542 | GCA_000618565.1 | 1446677 | 562 | Escherichia coli O157:H7 str. K2845 | K2845 | Proteobacteria | JHIQ01000033.1 |
| 1543 | GCA_000618585.1 | 1446678 | 562 | Escherichia coli O157:H7 str. K2854 | K2854 | Proteobacteria | JHIP01000003.1 |
| 1544 | GCA_000618605.1 | 1446679 | 562 | Escherichia coli O157:H7 str. K4396 | K4396 | Proteobacteria | JHIO01000002.1 |
| 1545 | GCA_000618625.1 | 1446681 | 562 | Escherichia coli O157:H7 str. K4406 | K4406 | Proteobacteria | JHIM01000001.1 |
| 1546 | GCA_000618665.1 | 1446674 | 562 | Escherichia coli O157:H7 str. K2324 | K2324 | Proteobacteria | JHIT01000105.1 |
| 1547 | GCA_000618685.1 | 1446682 | 562 | Escherichia coli O157:H7 str. K4527 | K4527 | Proteobacteria | JHIL01000001.1 |
| 1548 | GCA_000618745.1 | 1446683 | 562 | Escherichia coli O157:H7 str. K5418 | K5418 | Proteobacteria | JHII01000001.1 |
| 1549 | GCA_000618765.1 | 1446684 | 562 | Escherichia coli O157:H7 str. K5448 | K5448 | Proteobacteria | JHIH01000007.1 |
| 1550 | GCA_000618785.1 | 1446685 | 562 | Escherichia coli O157:H7 str. K5449 | K5449 | Proteobacteria | JHIG01000002.1 |
| 1551 | GCA_000618805.1 | 1446686 | 562 | Escherichia coli O157:H7 str. K5453 | K5453 | Proteobacteria | JHIF01000066.1 |
| 1552 | GCA_000618825.1 | 1446688 | 562 | Escherichia coli O157:H7 str. K5467 | K5467 | Proteobacteria | JHID01000001.1 |
| 1553 | GCA_000618845.1 | 1446689 | 562 | Escherichia coli O157:H7 str. K5602 | K5602 | Proteobacteria | JHIC01000015.1 |
| 1554 | GCA_000618865.1 | 1446690 | 562 | Escherichia coli O157:H7 str. K5607 | K5607 | Proteobacteria | JHIB01000088.1 |
| 1555 | GCA_000618885.1 | 1446687 | 562 | Escherichia coli O157:H7 str. K5460 | K5460 | Proteobacteria | JHIE01000002.1 |
| 1556 | GCA_000618925.2 | 1446697 | 562 | Escherichia coli O157:H7 str. K7140 | K7140 | Proteobacteria | JHHK01000016.1 |
| 1557 | GCA_000618945.2 | 1446649 | 562 | Escherichia coli O157:H7 str. 2011EL-2290 | 2011EL-2290 | Proteobacteria | JHKC01000008.1 |
| 1558 | GCA_000618985.2 | 1446651 | 562 | Escherichia coli O157:H7 str. 2011EL-2313 | 2011EL-2313 | Proteobacteria | JHKA01000028.1 |
| 1559 | GCA_000619045.2 | 1446617 | 562 | Escherichia coli O157:H7 str. 2009C-4258 | 2009C-4258 | Proteobacteria | JHGQ01000007.1 |
| 1560 | GCA_000619145.1 | 1446618 | 562 | Escherichia coli O157:H7 str. 2009EL1449 | 2009EL1449 | Proteobacteria | JHGF01000005.1 |
| 1561 | GCA_000619165.1 | 1446620 | 562 | Escherichia coli O157:H7 str. 2009EL1913 | 2009EL1913 | Proteobacteria | JHGD01000001.1 |
| 1562 | GCA_000619185.1 | 1446588 | 562 | Escherichia coli O145:NM str. 2010C-3508 | 2010C-3508 | Proteobacteria | JHFV01000110.1 |
| 1563 | GCA_000619365.1 | 1446693 | 562 | Escherichia coli O157:H7 str. K5852 | K5852 | Proteobacteria | JHHY01000003.1 |
| 1564 | GCA_000619385.1 | 1446694 | 562 | Escherichia coli O157:H7 str. K6590 | K6590 | Proteobacteria | JHHX01000008.1 |
| 1565 | GCA_000619405.1 | 1446695 | 562 | Escherichia coli O157:H7 str. K6676 | K6676 | Proteobacteria | JHHW01000001.1 |
| 1566 | GCA_000619425.1 | 1446696 | 562 | Escherichia coli O157:H7 str. K6687 | K6687 | Proteobacteria | JHHV01000121.1 |
| 1567 | GCA_000619445.1 | 1446691 | 562 | Escherichia coli O157:H7 str. K5609 | K5609 | Proteobacteria | JHIA01000021.1 |
| 1568 | GCA_000619665.1 | 1446616 | 562 | Escherichia coli O157:H7 str. 08-4529 | 08-4529 | Proteobacteria | JHHI01000009.1 |
| 1569 | GCA_000619685.2 | 1446698 | 562 | Escherichia coli O157:NM str. 08-4540 | 08-4540 | Proteobacteria | JHHH01000020.1 |
| 1570 | GCA_000627435.1 | 1444135 | 562 | Escherichia coli 1-250-04_S3_C1 | 1-250-04_S3_C1 | Proteobacteria | JJLU01000053.1 |
| 1571 | GCA_000627455.1 | 1444162 | 562 | Escherichia coli 1-182-04_S3_C2 | 1-182-04_S3_C2 | Proteobacteria | JJLT01000250.1 |
| 1572 | GCA_000627475.1 | 1444191 | 562 | Escherichia coli 1-182-04_S3_C3 | 1-182-04_S3_C3 | Proteobacteria | JJLS01000230.1 |
| 1573 | GCA_000627965.1 | 1444134 | 562 | Escherichia coli 1-182-04_S3_C1 | 1-182-04_S3_C1 | Proteobacteria | JJLV01000176.1 |
| 1574 | GCA_000632555.1 | 1446606 | 562 | Escherichia coli O157: str. 2010EL-2044 | 2010EL-2044 | Proteobacteria | JHME01000103.1 |
| 1575 | GCA_000632655.1 | 1446607 | 562 | Escherichia coli O157: str. 2010EL-2045 | 2010EL-2045 | Proteobacteria | JHMD01000001.1 |
| 1576 | GCA_000671295.1 | 1248823 | 562 | Escherichia coli O145:H28 str. RM12581 | RM12581 | Proteobacteria | CP007136.1 |
| 1577 | GCA_000687105.1 | 1444147 | 562 | Escherichia coli 2-005-03_S3_C2 | 2-005-03_S3_C2 | Proteobacteria | JMGT01000075.1 |
| 1578 | GCA_000687125.1 | 1444037 | 562 | Escherichia coli 2-011-08_S1_C1 | 2-011-08_S1_C1 | Proteobacteria | JMGQ01000091.1 |
| 1579 | GCA_000695155.1 | 1078035 | 562 | Escherichia coli O145:H28 str. 4865/96 | 4865/96 | Proteobacteria | JHEY01000012.1 |
| 1580 | GCA_000695175.1 | 562 | 562 | Escherichia coli | MI10-01 | Proteobacteria | JHEZ01000002.1 |
| 1581 | GCA_000700285.1 | 1444059 | 562 | Escherichia coli 3-475-03_S1_C1 | 3-475-03_S1_C1 | Proteobacteria | JNMJ01000048.1 |
| 1582 | GCA_000704005.1 | 1444151 | 562 | Escherichia coli 2-177-06_S3_C2 | 2-177-06_S3_C2 | Proteobacteria | JNQF01000030.1 |
| 1583 | GCA_000711415.1 | 1444071 | 562 | Escherichia coli 2-316-03_S1_C2 | 2-316-03_S1_C2 | Proteobacteria | JOMX01000159.1 |
| 1584 | GCA_000711475.1 | 1444042 | 562 | Escherichia coli 2-316-03_S1_C1 | 2-316-03_S1_C1 | Proteobacteria | JOMW01000194.1 |
| 1585 | GCA_000713175.1 | 1444281 | 562 | Escherichia coli 6-175-07_S4_C3 | 6-175-07_S4_C3 | Proteobacteria | JORK01000060.1 |
| 1586 | GCA_000713255.1 | 1444227 | 562 | Escherichia coli 6-537-08_S4_C1 | 6-537-08_S4_C1 | Proteobacteria | JORS01000112.1 |
| 1587 | GCA_000713605.1 | 1444257 | 562 | Escherichia coli 6-537-08_S4_C2 | 6-537-08_S4_C2 | Proteobacteria | JOSG01000027.1 |
| 1588 | GCA_000713745.1 | 1444179 | 562 | Escherichia coli 2-177-06_S3_C3 | 2-177-06_S3_C3 | Proteobacteria | JOSO01000008.1 |
| 1589 | GCA_000713815.1 | 1444163 | 562 | Escherichia coli 1-250-04_S3_C2 | 1-250-04_S3_C2 | Proteobacteria | JOSR01000197.1 |
| 1590 | GCA_000713905.1 | 1444087 | 562 | Escherichia coli 3-475-03_S1_C2 | 3-475-03_S1_C2 | Proteobacteria | JOQC01000025.1 |
| 1591 | GCA_000713985.1 | 1444196 | 562 | Escherichia coli 4-203-08_S3_C3 | 4-203-08_S3_C3 | Proteobacteria | JOQH01000040.1 |
| 1592 | GCA_000714055.1 | 1444171 | 562 | Escherichia coli 4-203-08_S3_C2 | 4-203-08_S3_C2 | Proteobacteria | JOQK01000042.1 |
| 1593 | GCA_000714385.1 | 1444226 | 562 | Escherichia coli 6-175-07_S4_C1 | 6-175-07_S4_C1 | Proteobacteria | JORB01000108.1 |
| 1594 | GCA_000714415.1 | 1444255 | 562 | Escherichia coli 6-175-07_S4_C2 | 6-175-07_S4_C2 | Proteobacteria | JORC01000054.1 |
| 1595 | GCA_000730345.1 | 1328859 | 562 | Escherichia coli O157:H7 str. SS17 | SS17 | Proteobacteria | CP008805.1 |
| 1596 | GCA_000732965.1 | 155864 | 562 | Escherichia coli O157:H7 str. EDL933 | EDL933 | Proteobacteria | CP008957.1 |
| 1597 | GCA_000797775.1 | 562 | 562 | Escherichia coli | CVM N34322PS | Proteobacteria | JUCY01000038.1 |
| 1598 | GCA_000798235.1 | 562 | 562 | Escherichia coli | CVM N36834PS | Proteobacteria | JUCF01000078.1 |
| 1599 | GCA_000798415.1 | 562 | 562 | Escherichia coli | CVM N34557PS | Proteobacteria | JUCU01000037.1 |
| 1600 | GCA_000803705.1 | 1330457 | 562 | Escherichia coli O157:H7 str. SS52 | SS52 | Proteobacteria | CP010304.1 |
| 1601 | GCA_000819265.1 | 562 | 562 | Escherichia coli | CVM N38428PS | Proteobacteria | JWYX01000074.1 |
| 1602 | GCA_000934865.1 | 562 | 562 | Escherichia coli | ATCC 35150 | Proteobacteria | JXUS01000117.1 |
| 1603 | GCA_000935075.1 | 562 | 562 | Escherichia coli | ATCC 35150 NalR | Proteobacteria | JYIO01000082.1 |
| 1604 | GCA_000953765.1 | 562 | 562 | Escherichia coli | MCI_507 | Proteobacteria | JZSD01000085.1 |
| 1605 | GCA_000965565.1 | 562 | 562 | Escherichia coli | St. Olav172 | Proteobacteria | JZDY01000017.1 |
| 1606 | GCA_000965655.1 | 562 | 562 | Escherichia coli | St. Olav173 | Proteobacteria | JZDZ01000061.1 |
| 1607 | GCA_000978815.1 | 83334 | 562 | Escherichia coli O157:H7 | 7.1_Anguil | Proteobacteria | LAZD01000130.1 |
| 1608 | GCA_000978845.1 | 83334 | 562 | Escherichia coli O157:H7 | Rafaela_II | Proteobacteria | LAYW01000314.1 |
| 1609 | GCA_000986635.1 | 562 | 562 | Escherichia coli | C1-057 | Proteobacteria | LAZO01000001.1 |
| 1610 | GCA_001006425.1 | 83334 | 562 | Escherichia coli O157:H7 | HB74 | Proteobacteria | LCWU01000234.1 |
| 1611 | GCA_001012075.1 | 562 | 562 | Escherichia coli | CFSAN026805 | Proteobacteria | LDCU01000029.1 |
| 1612 | GCA_001191125.1 | 562 | 562 | Escherichia coli | CFSAN026804 | Proteobacteria | LGZD01000035.1 |
| 1613 | GCA_001191185.1 | 562 | 562 | Escherichia coli | CFSAN026847 | Proteobacteria | LGZE01000020.1 |
| 1614 | GCA_001191195.1 | 562 | 562 | Escherichia coli | CFSAN026846 | Proteobacteria | LGZI01000146.1 |
| 1615 | GCA_001191525.1 | 562 | 562 | Escherichia coli | CFSAN026848 | Proteobacteria | LHAB01000019.1 |
| 1616 | GCA_001277695.1 | 562 | 562 | Escherichia coli | 503225_aEPEC | Proteobacteria | CYAV01000099.1 |
| 1617 | GCA_001281685.1 | 562 | 562 | Escherichia coli | STEC 771 | Proteobacteria | LGAZ01000005.1 |
| 1618 | GCA_001281725.1 | 562 | 562 | Escherichia coli | STEC 2868 | Proteobacteria | LGBJ01000009.1 |
| 1619 | GCA_001281755.1 | 562 | 562 | Escherichia coli | E09/224 | Proteobacteria | LGBP01000024.1 |
| 1620 | GCA_001281775.1 | 562 | 562 | Escherichia coli | STEC 989 | Proteobacteria | LGBA01000013.1 |
| 1621 | GCA_001281795.1 | 562 | 562 | Escherichia coli | STEC 994 | Proteobacteria | LGBB01000045.1 |
| 1622 | GCA_001281815.1 | 562 | 562 | Escherichia coli | STEC 1109 | Proteobacteria | LGBC01000012.1 |
| 1623 | GCA_001281845.1 | 562 | 562 | Escherichia coli | STEC 2075 | Proteobacteria | LGBD01000011.1 |
| 1624 | GCA_001281855.1 | 562 | 562 | Escherichia coli | STEC 2112 | Proteobacteria | LGBE01000001.1 |
| 1625 | GCA_001281885.1 | 562 | 562 | Escherichia coli | STEC 2257 | Proteobacteria | LGBF01000023.1 |
| 1626 | GCA_001281905.1 | 562 | 562 | Escherichia coli | STEC 2410 | Proteobacteria | LGBG01000006.1 |
| 1627 | GCA_001281915.1 | 562 | 562 | Escherichia coli | STEC 2821 | Proteobacteria | LGBI01000140.1 |
| 1628 | GCA_001281925.1 | 562 | 562 | Escherichia coli | STEC 2667 | Proteobacteria | LGBH01000007.1 |
| 1629 | GCA_001281965.1 | 562 | 562 | Escherichia coli | E09/10 | Proteobacteria | LGBK01000008.1 |
| 1630 | GCA_001281985.1 | 562 | 562 | Escherichia coli | EPEC 287 | Proteobacteria | LGBL01000025.1 |
| 1631 | GCA_001281995.1 | 562 | 562 | Escherichia coli | EPEC 393 | Proteobacteria | LGBM01000019.1 |
| 1632 | GCA_001282025.1 | 562 | 562 | Escherichia coli | EPEC 1572 | Proteobacteria | LGBN01000001.1 |
| 1633 | GCA_001282035.1 | 562 | 562 | Escherichia coli | EPEC 1669 | Proteobacteria | LGBO01000067.1 |
| 1634 | GCA_001282065.1 | 562 | 562 | Escherichia coli | STEC 2820 | Proteobacteria | LGBQ01000003.1 |
| 1635 | GCA_001297975.1 | 562 | 562 | Escherichia coli | STEC 343 | Proteobacteria | LDOZ01000007.1 |
| 1636 | GCA_001297985.1 | 562 | 562 | Escherichia coli | STEC 623 | Proteobacteria | LFUB01000011.1 |
| 1637 | GCA_001297995.1 | 562 | 562 | Escherichia coli | STEC 605 | Proteobacteria | LFUA01000020.1 |
| 1638 | GCA_001307215.1 | 83334 | 562 | Escherichia coli O157:H7 | WS4202 | Proteobacteria | CP012802.1 |
| 1639 | GCA_001309715.1 | 562 | 562 | Escherichia coli | PARC447 | Proteobacteria | LECJ01000192.1 |
| 1640 | GCA_001309815.1 | 562 | 562 | Escherichia coli | 03-6430 | Proteobacteria | LEAB01000117.1 |
| 1641 | GCA_001309835.1 | 562 | 562 | Escherichia coli | PARC449 | Proteobacteria | LEAC01000125.1 |
| 1642 | GCA_001309885.1 | 562 | 562 | Escherichia coli | CO6CE900 | Proteobacteria | LEAE01000148.1 |
| 1643 | GCA_001309895.1 | 562 | 562 | Escherichia coli | CO6CE1353 | Proteobacteria | LEAF01000141.1 |
| 1644 | GCA_001309905.1 | 562 | 562 | Escherichia coli | CO6CE1943 | Proteobacteria | LEAG01000046.1 |
| 1645 | GCA_001309925.1 | 562 | 562 | Escherichia coli | CO6CE2940 | Proteobacteria | LEAH01000084.1 |
| 1646 | GCA_001309965.1 | 562 | 562 | Escherichia coli | CO283 | Proteobacteria | LEAI01000013.1 |
| 1647 | GCA_001309985.1 | 562 | 562 | Escherichia coli | 1935 | Proteobacteria | LEAD01000163.1 |
| 1648 | GCA_001309995.1 | 562 | 562 | Escherichia coli | E0122 | Proteobacteria | LEAJ01000145.1 |
| 1649 | GCA_001310005.1 | 562 | 562 | Escherichia coli | LCDC7236 | Proteobacteria | LEAK01000079.1 |
| 1650 | GCA_001440045.1 | 83334 | 562 | Escherichia coli O157:H7 | G10 | Proteobacteria | LMXL01000097.1 |
| 1651 | GCA_001484305.1 | 562 | 562 | Escherichia coli | AY63 | Proteobacteria | LLYF01000048.1 |
| 1652 | GCA_001484355.1 | 562 | 562 | Escherichia coli | AY71 | Proteobacteria | LLYJ01000063.1 |
| 1653 | GCA_001519775.1 | 562 | 562 | Escherichia coli | GN02140 | Proteobacteria | LQSF01000099.1 |
| 1654 | GCA_001519815.1 | 562 | 562 | Escherichia coli | GN02298 | Proteobacteria | LQSX01000125.1 |
| 1655 | GCA_001519835.1 | 562 | 562 | Escherichia coli | GN02290 | Proteobacteria | LQSW01000169.1 |
| 1656 | GCA_001520115.1 | 562 | 562 | Escherichia coli | GN02343 | Proteobacteria | LQTC01000029.1 |
| 1657 | GCA_001520235.1 | 562 | 562 | Escherichia coli | GN02514 | Proteobacteria | LQUF01000146.1 |
| 1658 | GCA_001520275.1 | 562 | 562 | Escherichia coli | GN02359 | Proteobacteria | LQTG01000260.1 |
| 1659 | GCA_001520435.1 | 562 | 562 | Escherichia coli | GN02430 | Proteobacteria | LQTU01000172.1 |
| 1660 | GCA_001520495.1 | 562 | 562 | Escherichia coli | GN02426 | Proteobacteria | LQTS01000163.1 |
| 1661 | GCA_001520575.1 | 562 | 562 | Escherichia coli | GN02674 | Proteobacteria | LQVA01000248.1 |
| 1662 | GCA_001520835.1 | 562 | 562 | Escherichia coli | GN02707 | Proteobacteria | LQVE01000001.1 |
| 1663 | GCA_001520935.1 | 562 | 562 | Escherichia coli | GN03218 | Proteobacteria | LQVQ01000300.1 |
| 1664 | GCA_001520995.1 | 562 | 562 | Escherichia coli | GN02953 | Proteobacteria | LQVM01000164.1 |
| 1665 | GCA_001558995.1 | 562 | 562 | Escherichia coli | JEONG-1266 | Proteobacteria | CP014314.1 |
| 1666 | GCA_001571675.1 | 562 | 562 | Escherichia coli | UC5 | Proteobacteria | LRXT01000038.1 |
| 1667 | GCA_001575305.1 | 562 | 562 | Escherichia coli | G19 | Proteobacteria | LONY01000024.1 |
| 1668 | GCA_001576035.1 | 562 | 562 | Escherichia coli | G27 | Proteobacteria | LOOY01000033.1 |
| 1669 | GCA_001576325.1 | 562 | 562 | Escherichia coli | G28 | Proteobacteria | LOOZ01000046.1 |
| 1670 | GCA_000607265.1 | 1409787 | 571 | Klebsiella oxytoca G54 | G54 | Proteobacteria | KK097710.1 |
| 1671 | GCA_001078255.1 | 883124 | 571 | Klebsiella oxytoca 10-5249 | 10-5249 | Proteobacteria | KQ235791.1 |
| 1672 | GCA_000493135.1 | 1328363 | 573 | Klebsiella pneumoniae MGH 18 | MGH 18 | Proteobacteria | KI535680.1 |
| 1673 | GCA_000694715.1 | 1438797 | 573 | Klebsiella pneumoniae MGH 74 | MGH 74 | Proteobacteria | KK737643.1 |
| 1674 | GCA_001033505.1 | 573 | 573 | Klebsiella pneumoniae | MGH84 | Proteobacteria | KQ089360.1 |
| 1675 | GCA_001441285.1 | 573 | 573 | Klebsiella pneumoniae | B86 | Proteobacteria | UCD01000021.1 |
| 1676 | GCA_001536385.1 | 615 | 615 | Serratia marcescens | 2880STDY5682927 | Proteobacteria | FCJI01000002.1 |
| 1677 | GCA_001536405.1 | 615 | 615 | Serratia marcescens | 2880STDY5682928 | Proteobacteria | FCJJ01000003.1 |
| 1678 | GCA_001536505.1 | 615 | 615 | Serratia marcescens | 2880STDY5682857 | Proteobacteria | FCHL01000002.1 |
| 1679 | GCA_001536765.1 | 615 | 615 | Serratia marcescens | 2880STDY5682859 | Proteobacteria | FCHI01000002.1 |
| 1680 | GCA_001536965.1 | 615 | 615 | Serratia marcescens | 2880STDY5682942 | Proteobacteria | FCJX01000002.1 |
| 1681 | GCA_001537105.1 | 615 | 615 | Serratia marcescens | 2880STDY5683024 | Proteobacteria | FCGC01000003.1 |
| 1682 | GCA_001537305.1 | 615 | 615 | Serratia marcescens | 2880STDY5682930 | Proteobacteria | FCML01000002.1 |
| 1683 | GCA_001538765.1 | 615 | 615 | Serratia marcescens | 2880STDY5682826 | Proteobacteria | FCGQ01000002.1 |
| 1684 | GCA_001538825.1 | 615 | 615 | Serratia marcescens | 2880STDY5682883 | Proteobacteria | FCIK01000002.1 |
| 1685 | GCA_001539245.1 | 615 | 615 | Serratia marcescens | 2880STDY5682921 | Proteobacteria | FCJF01000002.1 |
| 1686 | GCA_001539385.1 | 615 | 615 | Serratia marcescens | 2880STDY5682913 | Proteobacteria | FCIZ01000003.1 |
| 1687 | GCA_001539425.1 | 615 | 615 | Serratia marcescens | 2880STDY5682877 | Proteobacteria | FCIC01000003.1 |
| 1688 | GCA_001539605.1 | 615 | 615 | Serratia marcescens | 2880STDY5682984 | Proteobacteria | FCLJ01000003.1 |
| 1689 | GCA_001539765.1 | 615 | 615 | Serratia marcescens | 2880STDY5682858 | Proteobacteria | FCHM01000002.1 |
| 1690 | GCA_001539905.1 | 615 | 615 | Serratia marcescens | 2880STDY5682961 | Proteobacteria | FCKL01000002.1 |
| 1691 | GCA_001540085.1 | 615 | 615 | Serratia marcescens | 2880STDY5682925 | Proteobacteria | FCMJ01000002.1 |
| 1692 | GCA_001540185.1 | 615 | 615 | Serratia marcescens | 2880STDY5682832 | Proteobacteria | FCGT01000003.1 |
| 1693 | GCA_001220145.1 | 630 | 630 | Yersinia enterocolitica | YE38/03 | Proteobacteria | CTKL01000007.1 |
| 1694 | GCA_001116865.1 | 631 | 631 | Yersinia intermedia | 58735 | Proteobacteria | CQER01000002.1 |
| 1695 | GCA_001010935.1 | 663 | 663 | Vibrio alginolyticus | V2 | Proteobacteria | LCSG01000013.1 |
| 1696 | GCA_001262735.1 | 669 | 669 | Vibrio harveyi | VH2 | Proteobacteria | LGYS01000041.1 |
| 1697 | GCA_001471575.1 | 669 | 669 | Vibrio harveyi | LMG 4044 | Proteobacteria | LOSK01000001.1 |
| 1698 | GCA_000707125.1 | 670 | 670 | Vibrio parahaemolyticus | CFSAN007445 | Proteobacteria | JNTC01000018.1 |
| 1699 | GCA_000786865.1 | 670 | 670 | Vibrio parahaemolyticus | 09.5357 | Proteobacteria | JTGT01000603.1 |
| 1700 | GCA_000877715.1 | 1288794 | 670 | Vibrio parahaemolyticus EN9701072 | EN9701072 | Proteobacteria | AOPI01000067.1 |
| 1701 | GCA_000877755.1 | 1288789 | 670 | Vibrio parahaemolyticus 3644 | 3644 | Proteobacteria | AOPD01000386.1 |
| 1702 | GCA_000960655.1 | 670 | 670 | Vibrio parahaemolyticus | 09-3217 | Proteobacteria | JZAQ01000061.1 |
| 1703 | GCA_001006195.1 | 670 | 670 | Vibrio parahaemolyticus | 10-4255 | Proteobacteria | JYJT01000016.1 |
| 1704 | GCA_001268045.1 | 670 | 670 | Vibrio parahaemolyticus | S456-5 | Proteobacteria | LFWN01000091.1 |
| 1705 | GCA_001270155.1 | 670 | 670 | Vibrio parahaemolyticus | S439-9 | Proteobacteria | LFZA01000015.1 |
| 1706 | GCA_001273575.1 | 670 | 670 | Vibrio parahaemolyticus | ISF-29-3 | Proteobacteria | LFYM01000016.1 |
| 1707 | GCA_001559805.1 | 670 | 670 | Vibrio parahaemolyticus | A4EZ700 | Proteobacteria | LOBT01000012.1 |
| 1708 | GCA_001595575.1 | 670 | 670 | Vibrio parahaemolyticus | A4EZ724 | Proteobacteria | LOHO01000038.1 |
| 1709 | GCA_000338875.1 | 1259812 | 674 | Vibrio mimicus CAIM 602 | CAIM 602 | Proteobacteria | AOMO01000034.1 |
| 1710 | GCA_000154025.1 | 410291 | 680 | Vibrio campbellii HY01 | HY01 | Proteobacteria | DS179487.1 |
| 1711 | GCA_000178495.1 | 228399 | 715 | Actinobacillus pleuropneumoniae serovar 1 str. 4074 | 4074 | Proteobacteria | ADOD01000018.1 |
| 1712 | GCA_000178575.1 | 754258 | 715 | Actinobacillus pleuropneumoniae serovar 9 str. CVJ13261 | CVJ13261 | Proteobacteria | ADOI01000037.1 |
| 1713 | GCA_000178615.1 | 754260 | 715 | Actinobacillus pleuropneumoniae serovar 11 str. 56153 | 56153 | Proteobacteria | ADOK01000019.1 |
| 1714 | GCA_000011785.1 | 205914 | 731 | Haemophilus somnus 129PT | 129PT | Proteobacteria | CP000436.1 |
| 1715 | GCA_000598725.1 | 1339306 | 817 | Bacteroides fragilis str. 3719 T6 | 3719 T6 | Bacteroidetes | JGEH01000041.1 |
| 1716 | GCA_000598745.1 | 1339324 | 817 | Bacteroides fragilis str. S24L26 | S24L26 | Bacteroidetes | JGEN01000046.1 |
| 1717 | GCA_000599005.1 | 1339323 | 817 | Bacteroides fragilis str. S24L15 | S24L15 | Bacteroidetes | JGEM01000110.1 |
| 1718 | GCA_000599325.1 | 1339325 | 817 | Bacteroides fragilis str. S24L34 | S24L34 | Bacteroidetes | JGEO01000038.1 |
| 1719 | GCA_001286525.1 | 817 | 817 | Bacteroides fragilis | BE1 | Bacteroidetes | LN877293.1 |
| 1720 | GCA_000699745.1 | 1339341 | 823 | Parabacteroides distasonis str. 3776 Po2 i | 3776 Po2 i | Bacteroidetes | JNHL01000154.1 |
| 1721 | GCA_000699785.1 | 1339342 | 823 | Parabacteroides distasonis str. 3776 D15 i | 3776 D15 i | Bacteroidetes | JNHK01000087.1 |
| 1722 | GCA_000699805.1 | 1339343 | 823 | Parabacteroides distasonis str. 3776 D15 iv | 3776 D15 iv | Bacteroidetes | JNHU01000002.1 |
| 1723 | GCA_001296085.1 | 76859 | 851 | Fusobacterium nucleatum subsp. animalis | KCOM 1279 | Fusobacteria | CP012713.1 |
| 1724 | GCA_001546435.1 | 851 | 851 | Fusobacterium nucleatum | MJR7757B | Fusobacteria | KQ956653.1 |
| 1725 | GCA_000163935.1 | 469621 | 860 | Fusobacterium periodonticum 1_1_41FAA | 1_1_41FAA | Fusobacteria | GG770381.1 |
| 1726 | GCA_000143225.1 | 757424 | 964 | Herbaspirillum seropedicae SmR1 | SmR1 | Proteobacteria | CP002039.1 |
| 1727 | GCA_001467505.1 | 28083 | 28083 | Legionella birminghamensis | CDC#1407-AL-14 | Proteobacteria | LNXT01000030.1 |
| 1728 | GCA_000300175.1 | 1004787 | 28108 | Alteromonas macleodii str. ‘Balearic Sea AD45’ | Balearic Sea AD45 | Proteobacteria | CP003873.1 |
| 1729 | GCA_000176155.1 | 675813 | 28172 | Vibrio metschnikovii CIP 69.14 | CIP 69.14 | Proteobacteria | ACZO01000003.1 |
| 1730 | GCA_000723805.1 | 1238449 | 28173 | Vibrio nigripulchritudo SO65 | SO65 | Proteobacteria | LK391963.1 |
| 1731 | GCA_001050595.1 | 1238430 | 28173 | Vibrio nigripulchritudo AM115 | AM115 | Proteobacteria | CANT01000061.1 |
| 1732 | GCA_001050655.1 | 1238441 | 28173 | Vibrio nigripulchritudo FTn2 | FTn2 | Proteobacteria | CANW01000040.1 |
| 1733 | GCA_001050675.1 | 1238445 | 28173 | Vibrio nigripulchritudo Pon4 | Pon4 | Proteobacteria | CAOA01000095.1 |
| 1734 | GCA_000764185.1 | 28229 | 28229 | Colwellia psychrerythraea | GAB14E | Proteobacteria | JQEC01000001.1 |
| 1735 | GCA_000182585.2 | 536230 | 28450 | Burkholderia pseudomallei MSHR346 | MSHR346 | Proteobacteria | ACOJ01000001.1 |
| 1736 | GCA_000756105.1 | 28450 | 28450 | Burkholderia pseudomallei | MSHR346 | Proteobacteria | CP008763.1 |
| 1737 | GCA_000757105.1 | 28450 | 28450 | Burkholderia pseudomallei | BDT | Proteobacteria | KN150939.1 |
| 1738 | GCA_000770435.1 | 1437000 | 28450 | Burkholderia pseudomallei MSHR1153 | MSHR1153 | Proteobacteria | CP009272.1 |
| 1739 | GCA_000774165.1 | 1435990 | 28450 | Burkholderia pseudomallei MSHR543 | MSHR543 | Proteobacteria | KN322990.1 |
| 1740 | GCA_000774325.1 | 1434206 | 28450 | Burkholderia pseudomallei MSHR4503 | MSHR4503 | Proteobacteria | KN323005.1 |
| 1741 | GCA_000774385.1 | 1434192 | 28450 | Burkholderia pseudomallei MSHR3951 | MSHR3951 | Proteobacteria | KN323013.1 |
| 1742 | GCA_000774475.1 | 1434927 | 28450 | Burkholderia pseudomallei MSHR3964 | MSHR3964 | Proteobacteria | KN323023.1 |
| 1743 | GCA_000774575.1 | 1434203 | 28450 | Burkholderia pseudomallei MSHR3960 | MSHR3960 | Proteobacteria | KN323029.1 |
| 1744 | GCA_000775095.1 | 1439861 | 28450 | Burkholderia pseudomallei TSV5 | TSV5 | Proteobacteria | JQGY01000129.1 |
| 1745 | GCA_000775105.1 | 1439859 | 28450 | Burkholderia pseudomallei TSV32 | TSV32 | Proteobacteria | JQHT01000033.1 |
| 1746 | GCA_000959185.1 | 1249475 | 28450 | Burkholderia pseudomallei MSHR840 | MSHR840 | Proteobacteria | CP009474.1 |
| 1747 | GCA_000696465.1 | 29471 | 29471 | Pectobacterium atrosepticum | JG10-08 | Proteobacteria | CP007744.1 |
| 1748 | GCA_000740965.1 | 29471 | 29471 | Pectobacterium atrosepticum | 21A | Proteobacteria | CP009125.1 |
| 1749 | GCA_001038685.1 | 1076138 | 29471 | Pectobacterium atrosepticum ICMP 1526 | ICMP 1526 | Proteobacteria | ALIV01000021.1 |
| 1750 | GCA_000764325.1 | 29495 | 29495 | Vibrio navarrensis | ATCC 51183 | Proteobacteria | JMCG01000001.1 |
| 1751 | GCA_000153205.1 | 314278 | 35797 | Nitrococcus mobilis Nb-231 | Nb-231 | Proteobacteria | CH672427.1 |
| 1752 | GCA_000238375.3 | 1117314 | 43655 | Pseudoalteromonas citrea DSM 8771 | NCIMB 1889 | Proteobacteria | AHBZ02000108.1 |
| 1753 | GCA_000238375.3 | 1117314 | 43655 | Pseudoalteromonas citrea DSM 8771 | NCIMB 1889 | Proteobacteria | AHBZ02000100.1 |
| 1754 | GCA_000238315.3 | 1117317 | 43662 | Pseudoalteromonas piscicida JCM 20779 | JCM 20779 | Proteobacteria | AHCC02000002.1 |
| 1755 | GCA_001467795.1 | 45067 | 45067 | Legionella lansingensis | ATCC 49751 | Proteobacteria | LNYI01000004.1 |
| 1756 | GCA_000175195.1 | 637911 | 51047 | Actinobacillus minor NM305 | NM305 | Proteobacteria | ACQL01000154.1 |
| 1757 | GCA_000807415.4 | 61645 | 61645 | Enterobacter asburiae | 35734 | Proteobacteria | CP012162.1 |
| 1758 | GCA_000956965.1 | 61645 | 61645 | Enterobacter asburiae | 35618 | Proteobacteria | JZZI01000011.1 |
| 1759 | GCA_001022755.1 | 61645 | 61645 | Enterobacter asburiae | GN02692 | Proteobacteria | LEDI01000011.1 |
| 1760 | GCA_000786255.1 | 66269 | 66269 | Pantoea stewartii | M009 | Proteobacteria | JRWI01000007.1 |
| 1761 | GCA_000803205.1 | 66269 | 66269 | Pantoea stewartii | M073a | Proteobacteria | JSXF01000009.1 |
| 1762 | GCA_000023545.1 | 579405 | 69223 | Dickeya paradisiaca Ech703 | Ech703 | Proteobacteria | CP001654.1 |
| 1763 | GCA_000153425.1 | 314280 | 74109 | Photobacterium profundum 3TCK | 3TCK | Proteobacteria | CH724134.1 |
| 1764 | GCA_001449005.1 | 75105 | 75105 | Paraburkholderia caribensis | MWAP64 | Proteobacteria | CP013103.1 |
| 1765 | GCA_000755725.1 | 95486 | 95486 | Burkholderia cenocepacia | DDS 22E-1 | Proteobacteria | CP007784.1 |
| 1766 | GCA_001467585.1 | 1212489 | 96228 | Legionella drozanskii LLAP-1 | ATCC 700990 | Proteobacteria | LNXY01000001.1 |
| 1767 | GCA_001527015.1 | 101571 | 101571 | Burkholderia ubonensis | MSMB1806WGS | Proteobacteria | LPBH01000095.1 |
| 1768 | GCA_001527185.1 | 101571 | 101571 | Burkholderia ubonensis | MSMB1800WGS | Proteobacteria | LPBC01000010.1 |
| 1769 | GCA_001528025.1 | 101571 | 101571 | Burkholderia ubonensis | MSMB2007WGS | Proteobacteria | LPBW01000043.1 |
| 1770 | GCA_001528325.1 | 101571 | 101571 | Burkholderia ubonensis | MSMB2013WGS | Proteobacteria | LPCC01000068.1 |
| 1771 | GCA_001528445.1 | 101571 | 101571 | Burkholderia ubonensis | MSMB2024WGS | Proteobacteria | LPCJ01000090.1 |
| 1772 | GCA_001529565.1 | 101571 | 101571 | Burkholderia ubonensis | MSMB2022WGS | Proteobacteria | LPCI01000021.1 |
| 1773 | GCA_001531605.1 | 101571 | 101571 | Burkholderia ubonensis | MSMB2104WGS | Proteobacteria | LPHJ01000073.1 |
| 1774 | GCA_001532205.1 | 101571 | 101571 | Burkholderia ubonensis | MSMB2110WGS | Proteobacteria | LPHP01000067.1 |
| 1775 | GCA_001532445.1 | 101571 | 101571 | Burkholderia ubonensis | MSMB2192WGS | Proteobacteria | LPIO01000050.1 |
| 1776 | GCA_001532455.1 | 101571 | 101571 | Burkholderia ubonensis | MSMB2191WGS | Proteobacteria | LPIN01000075.1 |
| 1777 | GCA_001532465.1 | 101571 | 101571 | Burkholderia ubonensis | MSMB2193WGS | Proteobacteria | LPIP01000082.1 |
| 1778 | GCA_001534475.1 | 101571 | 101571 | Burkholderia ubonensis | MSMB2019WGS | Proteobacteria | LPND01000106.1 |
| 1779 | GCA_001539165.1 | 101571 | 101571 | Burkholderia ubonensis | MSMB1490WGS | Proteobacteria | LPFP01000003.1 |
| 1780 | GCA_000769475.1 | 111105 | 111105 | Porphyromonas gulae | COT-052 OH3498 | Bacteroidetes | KN300394.1 |
| 1781 | GCA_000024725.1 | 472759 | 133539 | Nitrosococcus halophilus Nc 4 | Nc 4 | Proteobacteria | CP001798.1 |
| 1782 | GCA_000183725.1 | 709032 | 148813 | Sulfuricurvum kujiense DSM 16994 | DSM 16994 | Proteobacteria | CP002355.1 |
| 1783 | GCA_000412335.2 | 566551 | 158484 | Cedecea davisae DSM 4568 | DSM 4568 | Proteobacteria | KE161030.1 |
| 1784 | GCA_001596815.1 | 162157 | 162157 | Pseudoalteromonas spiralis | DSM 16099 | Proteobacteria | LVCN01000040.1 |
| 1785 | GCA_001465295.1 | 1315283 | 166935 | Pseudoalteromonas translucida KMM 520 | KMM 520 | Proteobacteria | CP011035.1 |
| 1786 | GCA_001274855.1 | 170661 | 170661 | Vibrio xuii | DSM 17185 | Proteobacteria | LHPK01000025.1 |
| 1787 | GCA_000755505.1 | 178606 | 178606 | Leptospirillum ferriphilum | DSM 14647 | Nitrospirae | JPGK01000006.1 |
| 1788 | GCA_000967895.1 | 698738 | 188908 | Oleispira antarctica RB-8 | | Proteobacteria | FO203512.1 |
| 1789 | GCA_000091465.1 | 575788 | 212663 | Vibrio tasmaniensis LGP32 | LGP32 | Proteobacteria | FM954972.2 |
| 1790 | GCA_001298715.1 | 235205 | 235205 | Bathymodiolus azoricus thioautotrophic gill symbiont | BazSymA | Proteobacteria | CDSC01000438.1 |
| 1791 | GCA_001048515.1 | 246167 | 246167 | Vibrio crassostreae | J5-20 | Proteobacteria | CCKB01000061.1 |
| 1792 | GCA_001368855.1 | 246167 | 246167 | Vibrio crassostreae | J2-9 | Proteobacteria | LK934305.1 |
| 1793 | GCA_000273235.1 | 997887 | 291644 | Bacteroides salyersiae CL02T12C01 | CL02T12C01 | Bacteroidetes | JH724309.1 |
| 1794 | GCA_000422245.1 | 1121439 | 293256 | Desulfovibrio alkalitolerans DSM 16529 | DSM 16529 | Proteobacteria | ATHI01000027.1 |
| 1795 | GCA_000310145.2 | 303682 | 303682 | Thauera sp. 28 | 28 | Proteobacteria | AMXA01000025.1 |
| 1796 | GCA_000153005.1 | 314290 | 314290 | Vibrio sp. MED222 | MED222 | Proteobacteria | CH902607.1 |
| 1797 | GCA_000241185.2 | 386429 | 386429 | Pseudoalteromonas sp. BSi20495 | BSi20495 | Proteobacteria | BADY01000203.1 |
| 1798 | GCA_000156355.1 | 637616 | 392484 | Methylophaga thiooxydans DMS010 | DMS010 | Proteobacteria | GG657892.1 |
| 1799 | GCA_001525985.1 | 488446 | 488446 | Burkholderia latens | RF32-BP12 | Proteobacteria | LOTQ01000014.1 |
| 1800 | GCA_000467165.1 | 1219077 | 512649 | Vibrio azureus NBRC 104587 | NBRC 104587 | Proteobacteria | BATL01000040.1 |
| 1801 | GCA_000179655.1 | 517433 | 517433 | Pantoea sp. aB | aB | Proteobacteria | AEDL01000002.1 |
| 1802 | GCA_000950415.1 | 553611 | 553611 | Photobacterium leiognathi | ATCC 25521 | Proteobacteria | JZSK01000008.1 |
| 1803 | GCA_000969825.1 | 1203610 | 574930 | Parabacteroides gordonii MS-1 | MS-1 | Bacteroidetes | KQ033921.1 |
| 1804 | GCA_000265075.1 | 1189621 | 590849 | Nitritalea halalkaliphila LW7 | LW7 | Bacteroidetes | AJYA01000079.1 |
| 1805 | GCA_000176215.1 | 675810 | 675810 | Vibrio sp. RC341 | RC341 | Proteobacteria | ACZT01000026.1 |
| 1806 | GCA_000176715.1 | 675815 | 675815 | Vibrio sp. RC586 | RC586 | Proteobacteria | ADBD01000008.1 |
| 1807 | GCA_000224655.1 | 693988 | 693988 | Bilophila sp. 4_1_30 | 4_1_30 | Proteobacteria | JH114231.1 |
| 1808 | GCA_000785575.1 | 701175 | 701175 | Vibrio caribbeanicus | T14 | Proteobacteria | JRWR01000004.1 |
| 1809 | GCA_000755345.1 | 754436 | 754436 | Photobacterium aphoticum | JCM 19237 | Proteobacteria | BBMN01000003.1 |
| 1810 | GCA_000320365.1 | 1230338 | 765840 | Moraxella macacae 0408225 | 0408225 | Proteobacteria | ANIN01000001.1 |
| 1811 | GCA_000211855.3 | 983544 | 983544 | Lacinutrix sp. 5H-3-7-4 | 5H-3-7-4 | Bacteroidetes | CP002825.1 |
| 1812 | GCA_000305755.2 | 992012 | 992012 | Vibrio sp. HENC-03 | HENC-03 | Proteobacteria | JH976122.1 |
| 1813 | GCA_000333895.2 | 1056512 | 1056512 | Grimontia indica | AK16 | Proteobacteria | ANFM02000072.1 |
| 1814 | GCA_000722635.1 | 1137799 | 1137799 | Endozoicomonas numazuensis | DSM 25634 | Proteobacteria | JOKH01000001.1 |
| 1815 | GCA_000276965.1 | 1177931 | 1177931 | Thiovulum sp. ES | ES | Proteobacteria | AKKQ01000015.1 |
| 1816 | GCA_001541335.1 | 1194427 | 1194427 | Vibrio toranzoniae | Vb 10.8 | Proteobacteria | LMXU01000011.1 |
| 1817 | GCA_000802255.1 | 1348852 | 1348852 | Mumia flava | MUSC 201 | Actinobacteria | JTDJ01000134.1 |
| 1818 | GCA_000743945.1 | 1072685 | 1472345 | Basilea psittacipulmonis DSM 24701 | DSM 24701 | Proteobacteria | CP009238.1 |
| 1819 | GCA_000830185.1 | 1539064 | 1539064 | Sulfurovum sp. FS06-10 | | Proteobacteria | JQIR01000120.1 |
| 1820 | GCA_000830175.1 | 1539065 | 1539065 | Sulfurovum sp. FS08-3 | | Proteobacteria | JQIS01000009.1 |
| 1821 | GCA_000292935.1 | 1216362 | 1583098 | Fusobacterium hwasookii ChDC F128 | ChDC F128 | Fusobacteria | ALVD01000001.1 |
| 1822 | GCA_000807225.1 | 1583100 | 1583100 | Myroides sp. A21 | A21 | Bacteroidetes | CP010327.1 |
| 1823 | GCA_001431295.1 | 1590043 | 1590043 | Coxiellaceae bacterium HT99 | HT99 | Proteobacteria | LKAJ01000001.1 |
| 1824 | GCA_001188205.1 | 1647102 | 1647102 | Vibrio sp. VPAP30 | VPAP30 | Proteobacteria | LBLS01000036.1 |
| 1825 | GCA_000695255.1 | 1654360 | 1654360 | Photobacterium galatheae | S2753 | Proteobacteria | JMIB01000002.1 |
| 1826 | GCA_001011075.1 | 1656220 | 1656220 | Serratia sp. TEL | TEL | Proteobacteria | LDEG01000006.1 |
| 1827 | GCA_001401805.1 | 1723758 | 1723758 | Pseudoalteromonas sp. P1-25 | P1-25 | Proteobacteria | LKDW01000016.1 |
| 1828 | GCA_001421885.1 | 1736221 | 1736221 | Pseudomonas sp. Leaf48 | Leaf48 | Proteobacteria | LMLH01000030.1 |
| 1829 | GCA_001544875.1 | 1777139 | 1777139 | Burkholderia sp. LMG 29321 | | Proteobacteria | FCOA01000023.1 |
| 1830 | GCA_001537265.1 | 1805637 | 1805637 | Serratia sp. 2880STDY5682895 | 2880STDY5682895 | Proteobacteria | FCJL01000002.1 |

(Part B)

| | | | | | | M | | | | Q | | | | U |
|---|---|---|---|---|---|---|---|---|---|---|---|---|---|---|
| | | | | | | ZorA | | | | ZorB | | | | ZorC |
| | | I | J | K | L | protein | N | O | P | protein | R | S | T | protein |
| | H | #genes | ZorA | PRT SEQ | DNA SEQ | length | ZorB | PRT SEQ | DNA SEQ | length | ZorC | PRT SEQ | DNA SEQ | length |
| 1 | Type | identified | protein_accession | ID NO: | ID NO: | (aa) | protein_accession | ID NO: | ID NO: | (aa) | protein_accession | ID NO: | ID NO: | (aa) |
| 2 | I | 3 | ABS66238.1 | 78 | 6649 | 704 | ABS66239.1 | 79 | 6650 | 296 | ABS66240.1 | 80 | 6651 | 469 |
| 3 | I | 4 | KGH31560.1 | 81 | 6652 | 705 | KGH31559.1 | 82 | 6653 | 248 | KGH31558.1 | 83 | 6654 | 575 |
| 4 | I | 4 | ABR84348.1 | 85 | 6656 | 708 | ABR81630.1 | 86 | 6657 | 254 | ABR85988.1 | 87 | 6658 | 648 |
| 5 | I | 4 | EIE46557.1 | 89 | 6660 | 708 | EIE46558.1 | 90 | 6661 | 254 | EIE46559.1 | 91 | 6662 | 670 |
| 6 | I | 4 | EKA35841.1 | 93 | 6664 | 731 | EKA35840.1 | 94 | 6665 | 245 | EKA35839.1 | 95 | 6666 | 528 |
| 7 | I | 4 | EKA51673.1 | 97 | 6668 | 731 | EKA51672.1 | 98 | 6669 | 245 | EKA51671.1 | 99 | 6670 | 528 |
| 8 | I | 4 | EOQ78914.1 | 101 | 6672 | 714 | EOQ78913.1 | 102 | 6673 | 245 | EOQ78912.1 | 103 | 6674 | 547 |
| 9 | I | 3 | EQL43276.1 | 105 | 6676 | 708 | EQL43277.1 | 106 | 6677 | 254 | EQL43278.1 | 107 | 6678 | 417 |
| 10 | I | 4 | ERY08923.1 | 108 | 6679 | 708 | ERY08922.1 | 109 | 6680 | 254 | ERY08921.1 | 110 | 6681 | 670 |
| 11 | I | 4 | ERY16790.1 | 112 | 6683 | 731 | ERY16789.1 | 113 | 6684 | 245 | ERY16788.1 | 114 | 6685 | 558 |
| 12 | I | 4 | ERY21334.1 | 116 | 6687 | 708 | ERY21333.1 | 117 | 6688 | 254 | ERY21332.1 | 118 | 6689 | 670 |
| 13 | I | 4 | ERV56557.1 | 120 | 6691 | 708 | ERV56556.1 | 121 | 6692 | 254 | ERV56555.1 | 122 | 6693 | 670 |
| 14 | I | 4 | ERX20611.1 | 124 | 6695 | 731 | ERX20610.1 | 125 | 6696 | 245 | ERX20609.1 | 126 | 6697 | 558 |
| 15 | I | 4 | ERX27344.1 | 128 | 6699 | 731 | ERX27343.1 | 129 | 6700 | 245 | ERX27342.1 | 130 | 6701 | 558 |
| 16 | I | 4 | ERZ22541.1 | 132 | 6703 | 731 | ERZ22540.1 | 133 | 6704 | 245 | ERZ22539.1 | 134 | 6705 | 558 |
| 17 | I | 4 | ERX75596.1 | 136 | 6707 | 731 | ERX75595.1 | 137 | 6708 | 245 | ERX75594.1 | 138 | 6709 | 558 |
| 18 | I | 4 | ESR70192.1 | 140 | 6711 | 731 | ESR70191.1 | 141 | 6712 | 245 | ESR70190.1 | 142 | 6713 | 558 |
| 19 | I | 4 | ETU86359.1 | 144 | 6715 | 708 | ETU86358.1 | 145 | 6716 | 254 | ETU86357.1 | 146 | 6717 | 670 |
| 20 | I | 4 | CDI94249.1 | 148 | 6719 | 708 | CDI94248.1 | 149 | 6720 | 254 | CDI94247.1 | 150 | 6721 | 255 |
| 21 | I | 4 | CDI95166.1 | 152 | 6723 | 731 | CDI95167.1 | 153 | 6724 | 245 | CDI95168.1 | 154 | 6725 | 547 |
| 22 | I | 4 | EVT82482.1 | 156 | 6727 | 708 | EVT82481.1 | 157 | 6728 | 254 | EVT82480.1 | 158 | 6729 | 663 |
| 23 | I | 4 | EYU03497.1 | 160 | 6731 | 731 | EYU03498.1 | 161 | 6732 | 245 | EYU03499.1 | 162 | 6733 | 558 |
| 24 | I | 4 | EZN45278.1 | 164 | 6735 | 781 | EZN45279.1 | 165 | 6736 | 247 | EZN45280.1 | 166 | 6737 | 723 |
| 25 | I | 4 | EZO27044.1 | 168 | 6739 | 708 | EZO27045.1 | 169 | 6740 | 254 | EZO27046.1 | 170 | 6741 | 640 |
| 26 | I | 4 | EZP05708.1 | 172 | 6743 | 708 | EZP05709.1 | 173 | 6744 | 254 | EZP05710.1 | 174 | 6745 | 640 |
| 27 | I | 4 | CDH74042.1 | 176 | 6747 | 708 | CDH74041.1 | 177 | 6748 | 254 | CDH74040.1 | 178 | 6749 | 670 |
| 28 | I | 4 | CDH70661.1 | 180 | 6751 | 731 | CDH70662.1 | 181 | 6752 | 245 | CDH70663.1 | 182 | 6753 | 547 |
| 29 | I | 4 | KEA10735.1 | 184 | 6755 | 708 | KEA10736.1 | 185 | 6756 | 254 | KEA10737.1 | 186 | 6757 | 670 |
| 30 | I | 4 | AJF50277.1 | 188 | 6759 | 731 | AJF50276.1 | 189 | 6760 | 245 | AJF50275.1 | 190 | 6761 | 547 |
| 31 | I | 4 | AKE69360.1 | 192 | 6763 | 731 | AKE69361.1 | 193 | 6764 | 245 | AKE69362.1 | 194 | 6765 | 547 |
| 32 | I | 4 | AKE72978.1 | 196 | 6767 | 708 | AKE72977.1 | 197 | 6768 | 254 | AKE72976.1 | 198 | 6769 | 670 |
| 33 | I | 4 | CRQ30662.1 | 200 | 6771 | 708 | CRQ30646.1 | 201 | 6772 | 254 | CRQ30630.1 | 202 | 6773 | 670 |
| 34 | I | 4 | CRQ14001.1 | 204 | 6775 | 731 | CRQ14037.1 | 205 | 6776 | 245 | CRQ14055.1 | 206 | 6777 | 547 |
| 35 | I | 4 | CRQ20930.1 | 208 | 6779 | 708 | CRQ20903.1 | 209 | 6780 | 254 | CRQ20878.1 | 210 | 6781 | 670 |
| 36 | I | 4 | CRP04583.1 | 212 | 6783 | 731 | CRP04548.1 | 213 | 6784 | 245 | CRP04515.1 | 214 | 6785 | 547 |
| 37 | I | 4 | CRQ05457.1 | 216 | 6787 | 731 | CRQ05470.1 | 217 | 6788 | 245 | CRQ05489.1 | 218 | 6789 | 547 |
| 38 | I | 4 | CRP94057.1 | 220 | 6791 | 708 | CRP94029.1 | 221 | 6792 | 254 | CRP94009.1 | 222 | 6793 | 670 |
| 39 | I | 4 | CRP94866.1 | 224 | 6795 | 708 | CRP94849.1 | 225 | 6796 | 254 | CRP94824.1 | 226 | 6797 | 670 |
| 40 | I | 4 | CRQ51053.1 | 228 | 6799 | 731 | CRQ51040.1 | 229 | 6800 | 245 | CRQ51031.1 | 230 | 6801 | 547 |
| 41 | I | 4 | CRS01042.1 | 232 | 6803 | 708 | CRS01058.1 | 233 | 6804 | 254 | CRS01074.1 | 234 | 6805 | 670 |
| 42 | I | 4 | CRX34600.1 | 236 | 6807 | 708 | CRX34599.1 | 237 | 6808 | 254 | CRX34598.1 | 238 | 6809 | 670 |
| 43 | I | 4 | CRX35811.1 | 240 | 6811 | 731 | CRX35810.1 | 241 | 6812 | 245 | CRX35809.1 | 242 | 6813 | 547 |
| 44 | I | 4 | CRN87107.1 | 244 | 6815 | 731 | CRN87087.1 | 245 | 6816 | 245 | CRN87069.1 | 246 | 6817 | 547 |
| 45 | I | 4 | CRQ28236.1 | 248 | 6819 | 731 | CRQ28254.1 | 249 | 6820 | 245 | CRQ28266.1 | 250 | 6821 | 547 |
| 46 | I | 4 | CRQ21539.1 | 252 | 6823 | 708 | CRQ21557.1 | 253 | 6824 | 254 | CRQ21576.1 | 254 | 6825 | 670 |
| 47 | I | 4 | CRN79596.1 | 256 | 6827 | 708 | CRN79621.1 | 257 | 6828 | 254 | CRN79649.1 | 258 | 6829 | 670 |
| 48 | I | 4 | CRO17466.1 | 260 | 6831 | 731 | CRO17426.1 | 261 | 6832 | 245 | CRO17380.1 | 262 | 6833 | 547 |
| 49 | I | 4 | CRW80533.1 | 264 | 6835 | 708 | CRW80527.1 | 265 | 6836 | 254 | CRW80520.1 | 266 | 6837 | 670 |
| 50 | I | 4 | CRW81746.1 | 268 | 6839 | 731 | CRW81753.1 | 269 | 6840 | 245 | CRW81763.1 | 270 | 6841 | 547 |
| 51 | I | 4 | CRS64348.1 | 272 | 6843 | 731 | CRS64342.1 | 273 | 6844 | 245 | CRS64337.1 | 274 | 6845 | 547 |
| 52 | I | 4 | CRS61289.1 | 276 | 6847 | 708 | CRS61288.1 | 277 | 6848 | 254 | CRS61287.1 | 278 | 6849 | 670 |
| 53 | I | 4 | CRX10287.1 | 280 | 6851 | 731 | CRX10279.1 | 281 | 6852 | 245 | CRX10272.1 | 282 | 6853 | 547 |
| 54 | I | 4 | CRX00557.1 | 284 | 6855 | 708 | CRX00549.1 | 285 | 6856 | 254 | CRX00541.1 | 286 | 6857 | 670 |
| 55 | I | 4 | CRW55933.1 | 288 | 6859 | 731 | CRW55913.1 | 289 | 6860 | 245 | CRW55893.1 | 290 | 6861 | 547 |
| 56 | I | 4 | CRX16431.1 | 292 | 6863 | 708 | CRX16423.1 | 293 | 6864 | 254 | CRX16416.1 | 294 | 6865 | 670 |
| 57 | I | 4 | CRP10808.1 | 296 | 6867 | 708 | CRP10838.1 | 297 | 6868 | 254 | CRP10876.1 | 298 | 6869 | 670 |
| 58 | I | 4 | CRP41275.1 | 300 | 6871 | 731 | CRP41308.1 | 301 | 6872 | 245 | CRP41345.1 | 302 | 6873 | 547 |
| 59 | I | 4 | CRP14749.1 | 304 | 6875 | 708 | CRP14725.1 | 305 | 6876 | 254 | CRP14706.1 | 306 | 6877 | 670 |
| 60 | I | 4 | CRP47753.1 | 308 | 6879 | 731 | CRP47733.1 | 309 | 6880 | 245 | CRP47704.1 | 310 | 6881 | 547 |
| 61 | I | 4 | CRO88348.1 | 312 | 6883 | 731 | CRO88299.1 | 313 | 6884 | 245 | CRO88247.1 | 314 | 6885 | 547 |
| 62 | I | 4 | CRO42842.1 | 316 | 6887 | 708 | CRO42772.1 | 317 | 6888 | 254 | CRO42732.1 | 318 | 6889 | 670 |
| 63 | I | 4 | CRN87606.1 | 320 | 6891 | 731 | CRN87577.1 | 321 | 6892 | 245 | CRN87556.1 | 322 | 6893 | 547 |
| 64 | I | 4 | CRN66199.1 | 324 | 6895 | 708 | CRN66218.1 | 325 | 6896 | 254 | CRN66246.1 | 326 | 6897 | 670 |
| 65 | I | 4 | CRP41066.1 | 328 | 6899 | 708 | CRP41088.1 | 329 | 6900 | 254 | CRP41112.1 | 330 | 6901 | 670 |
| 66 | I | 4 | CRP68499.1 | 332 | 6903 | 731 | CRP68477.1 | 333 | 6904 | 245 | CRP68458.1 | 334 | 6905 | 547 |
| 67 | I | 4 | CRP76330.1 | 336 | 6907 | 708 | CRP76294.1 | 337 | 6908 | 254 | CRP76259.1 | 338 | 6909 | 670 |
| 68 | I | 4 | CRQ24549.1 | 340 | 6911 | 731 | CRQ24529.1 | 341 | 6912 | 245 | CRQ24511.1 | 342 | 6913 | 547 |
| 69 | I | 4 | CRP82373.1 | 344 | 6915 | 731 | CRP82350.1 | 345 | 6916 | 245 | CRP82329.1 | 346 | 6917 | 547 |
| 70 | I | 4 | CRP61046.1 | 348 | 6919 | 708 | CRP61016.1 | 349 | 6920 | 254 | CRP60987.1 | 350 | 6921 | 670 |
| 71 | I | 4 | CRP29707.1 | 352 | 6923 | 731 | CRP29680.1 | 353 | 6924 | 245 | CRP29649.1 | 354 | 6925 | 547 |
| 72 | I | 4 | CRO50770.1 | 356 | 6927 | 708 | CRO50854.1 | 357 | 6928 | 254 | CRO50880.1 | 358 | 6929 | 670 |
| 73 | I | 3 | CRP19911.1 | 360 | 6931 | 731 | CRP19885.1 | 361 | 6932 | 245 | CRP19855.1 | 362 | 6933 | 547 |
| 74 | I | 4 | CRP88471.1 | 363 | 6934 | 708 | CRP88447.1 | 364 | 6935 | 254 | CRP88424.1 | 365 | 6936 | 670 |
| 75 | I | 4 | CRP42118.1 | 367 | 6938 | 708 | CRP42092.1 | 368 | 6939 | 254 | CRP42069.1 | 369 | 6940 | 670 |
| 76 | I | 4 | CRP91193.1 | 371 | 6942 | 731 | CRP91170.1 | 372 | 6943 | 245 | CRP91145.1 | 373 | 6944 | 547 |
| 77 | I | 4 | CRN61579.1 | 375 | 6946 | 708 | CRN61562.1 | 376 | 6947 | 254 | CRN61546.1 | 377 | 6948 | 670 |
| 78 | I | 4 | CRN91417.1 | 379 | 6950 | 731 | CRN91437.1 | 380 | 6951 | 245 | CRN91483.1 | 381 | 6952 | 547 |
| 79 | I | 4 | CRP44694.1 | 383 | 6954 | 731 | CRP44734.1 | 384 | 6955 | 245 | CRP44770.1 | 385 | 6956 | 547 |
| 80 | I | 4 | CRP16339.1 | 387 | 6958 | 708 | CRP16291.1 | 388 | 6959 | 254 | CRP16258.1 | 389 | 6960 | 670 |
| 81 | I | 4 | CRP57045.1 | 391 | 6962 | 708 | CRP57076.1 | 392 | 6963 | 254 | CRP57102.1 | 393 | 6964 | 670 |
| 82 | I | 4 | CRP83731.1 | 395 | 6966 | 731 | CRP83754.1 | 396 | 6967 | 245 | CRP83780.1 | 397 | 6968 | 547 |
| 83 | I | 4 | CRO13767.1 | 399 | 6970 | 708 | CRO13733.1 | 400 | 6971 | 254 | CRO13695.1 | 401 | 6972 | 670 |
| 84 | I | 4 | CRO55633.1 | 403 | 6974 | 731 | CRO55573.1 | 404 | 6975 | 245 | CRO55551.1 | 405 | 6976 | 547 |
| 85 | I | 4 | CRO41585.1 | 407 | 6978 | 731 | CRO41607.1 | 408 | 6979 | 245 | CRO41618.1 | 409 | 6980 | 547 |
| 86 | I | 4 | CRO12578.1 | 411 | 6982 | 708 | CRO12630.1 | 412 | 6983 | 254 | CRO12724.1 | 413 | 6984 | 670 |
| 87 | I | 4 | CRP86193.1 | 415 | 6986 | 731 | CRP86228.1 | 416 | 6987 | 245 | CRP86261.1 | 417 | 6988 | 547 |
| 88 | I | 4 | CRP43660.1 | 419 | 6990 | 708 | CRP43627.1 | 420 | 6991 | 254 | CRP43600.1 | 421 | 6992 | 670 |
| 89 | I | 4 | CRP83380.1 | 423 | 6994 | 731 | CRP83353.1 | 424 | 6995 | 245 | CRP83330.1 | 425 | 6996 | 547 |
| 90 | I | 4 | CRP15070.1 | 427 | 6998 | 708 | CRP15101.1 | 428 | 6999 | 254 | CRP15132.1 | 429 | 7000 | 670 |
| 91 | I | 4 | CRP60638.1 | 431 | 7002 | 708 | CRP60613.1 | 432 | 7003 | 254 | CRP60586.1 | 433 | 7004 | 670 |
| 92 | I | 4 | CRQ10821.1 | 435 | 7006 | 731 | CRQ10804.1 | 436 | 7007 | 245 | CRQ10786.1 | 437 | 7008 | 547 |
| 93 | I | 4 | CRP54994.1 | 439 | 7010 | 731 | CRP54888.1 | 440 | 7011 | 245 | CRP54862.1 | 441 | 7012 | 547 |
| 94 | I | 4 | CRP76342.1 | 443 | 7014 | 708 | CRP76468.1 | 444 | 7015 | 254 | CRP76497.1 | 445 | 7016 | 670 |
| 95 | I | 4 | CRP45935.1 | 447 | 7018 | 731 | CRP45955.1 | 448 | 7019 | 245 | CRP45983.1 | 449 | 7020 | 547 |
| 96 | I | 4 | CRP23202.1 | 451 | 7022 | 708 | CRP23176.1 | 452 | 7023 | 254 | CRP23144.1 | 453 | 7024 | 670 |
| 97 | I | 4 | CRP65371.1 | 455 | 7026 | 708 | CRP65407.1 | 456 | 7027 | 254 | CRP65439.1 | 457 | 7028 | 670 |
| 98 | I | 4 | CRP93880.1 | 459 | 7030 | 731 | CRP93850.1 | 460 | 7031 | 245 | CRP93825.1 | 461 | 7032 | 547 |
| 99 | I | 4 | CRQ27494.1 | 463 | 7034 | 708 | CRQ27510.1 | 464 | 7035 | 254 | CRQ27528.1 | 465 | 7036 | 670 |
| 100 | I | 4 | CRQ56582.1 | 467 | 7038 | 731 | CRQ56568.1 | 468 | 7039 | 245 | CRQ56555.1 | 469 | 7040 | 547 |
| 101 | I | 4 | CRP80430.1 | 471 | 7042 | 731 | CRP80455.1 | 472 | 7043 | 245 | CRP80479.1 | 473 | 7044 | 547 |
| 102 | I | 4 | CRP36068.1 | 475 | 7046 | 708 | CRP36044.1 | 476 | 7047 | 254 | CRP36016.1 | 477 | 7048 | 670 |
| 103 | I | 4 | CRO87098.1 | 479 | 7050 | 708 | CRO87131.1 | 480 | 7051 | 254 | CRO87170.1 | 481 | 7052 | 670 |
| 104 | I | 4 | CRP11508.1 | 483 | 7054 | 731 | CRP11471.1 | 484 | 7055 | 245 | CRP11443.1 | 485 | 7056 | 547 |
| 105 | I | 4 | CRP24964.1 | 487 | 7058 | 731 | CRP24937.1 | 488 | 7059 | 245 | CRP24910.1 | 489 | 7060 | 547 |
| 106 | I | 4 | CRO90638.1 | 491 | 7062 | 708 | CRO90673.1 | 492 | 7063 | 254 | CRO90698.1 | 493 | 7064 | 670 |
| 107 | I | 4 | CRP17905.1 | 495 | 7066 | 731 | CRP17871.1 | 496 | 7067 | 245 | CRP17834.1 | 497 | 7068 | 547 |
| 108 | I | 4 | CRO58804.1 | 499 | 7070 | 708 | CRO58845.1 | 500 | 7071 | 254 | CRO58885.1 | 501 | 7072 | 670 |
| 109 | I | 4 | CRP27039.1 | 503 | 7074 | 708 | CRP27013.1 | 504 | 7075 | 254 | CRP26989.1 | 505 | 7076 | 670 |
| 110 | I | 4 | CRP96092.1 | 507 | 7078 | 731 | CRP96112.1 | 508 | 7079 | 245 | CRP96135.1 | 509 | 7080 | 547 |
| 111 | I | 4 | CRN60140.1 | 511 | 7082 | 708 | CRN60167.1 | 512 | 7083 | 254 | CRN60197.1 | 513 | 7084 | 670 |
| 112 | I | 4 | CRO00267.1 | 515 | 7086 | 731 | CRO00240.1 | 516 | 7087 | 245 | CRO00221.1 | 517 | 7088 | 547 |
| 113 | I | 4 | CRO92615.1 | 519 | 7090 | 731 | CRO92586.1 | 520 | 7091 | 245 | CRO92548.1 | 521 | 7092 | 547 |
| 114 | I | 4 | CRO70145.1 | 523 | 7094 | 708 | CRO70186.1 | 524 | 7095 | 254 | CRO70227.1 | 525 | 7096 | 670 |
| 115 | I | 4 | CRP22981.1 | 527 | 7098 | 708 | CRP22953.1 | 528 | 7099 | 254 | CRP22919.1 | 529 | 7100 | 670 |
| 116 | I | 4 | CRP50679.1 | 531 | 7102 | 731 | CRP50614.1 | 532 | 7103 | 245 | CRP50550.1 | 533 | 7104 | 547 |
| 117 | I | 4 | CRO39184.1 | 535 | 7106 | 731 | CRO39213.1 | 536 | 7107 | 245 | CRO39248.1 | 537 | 7108 | 547 |
| 118 | I | 4 | CRO06753.1 | 539 | 7110 | 708 | CRO06788.1 | 540 | 7111 | 254 | CRO06822.1 | 541 | 7112 | 670 |
| 119 | I | 4 | CRP41965.1 | 543 | 7114 | 731 | CRP42029.1 | 544 | 7115 | 245 | CRP42062.1 | 545 | 7116 | 547 |
| 120 | I | 4 | CRP09067.1 | 547 | 7118 | 708 | CRP09046.1 | 548 | 7119 | 254 | CRP09026.1 | 549 | 7120 | 670 |
| 121 | I | 4 | CRQ57015.1 | 551 | 7122 | 731 | CRQ57008.1 | 552 | 7123 | 245 | CRQ57001.1 | 553 | 7124 | 547 |
| 122 | I | 4 | CRQ59344.1 | 555 | 7126 | 708 | CRQ59358.1 | 556 | 7127 | 254 | CRQ59370.1 | 557 | 7128 | 670 |
| 123 | I | 4 | CRQ91414.1 | 559 | 7130 | 731 | CRQ91399.1 | 560 | 7131 | 245 | CRQ91388.1 | 561 | 7132 | 547 |
| 124 | I | 4 | CRQ84133.1 | 563 | 7134 | 708 | CRQ84121.1 | 564 | 7135 | 254 | CRQ84110.1 | 565 | 7136 | 670 |
| 125 | I | 4 | CRQ52280.1 | 567 | 7138 | 731 | CRQ52265.1 | 568 | 7139 | 245 | CRQ52253.1 | 569 | 7140 | 547 |
| 126 | I | 4 | CRQ54273.1 | 571 | 7142 | 708 | CRQ54285.1 | 572 | 7143 | 254 | CRQ54295.1 | 573 | 7144 | 670 |
| 127 | I | 4 | CRQ62274.1 | 575 | 7146 | 731 | CRQ62260.1 | 576 | 7147 | 245 | CRQ62248.1 | 577 | 7148 | 547 |
| 128 | I | 4 | CRQ81707.1 | 579 | 7150 | 708 | CRQ81699.1 | 580 | 7151 | 254 | CRQ81693.1 | 581 | 7152 | 670 |
| 129 | I | 4 | CRQ17118.1 | 583 | 7154 | 708 | CRQ17143.1 | 584 | 7155 | 254 | CRQ17167.1 | 585 | 7156 | 670 |
| 130 | I | 3 | CRQ37524.1 | 587 | 7158 | 731 | | | | | CRQ37557.1 | 588 | 7159 | 547 |
| 131 | I | 4 | CRO85949.1 | 590 | 7161 | 731 | CRO85973.1 | 591 | 7162 | 245 | CRO86006.1 | 592 | 7163 | 547 |
| 132 | I | 4 | CRQ40696.1 | 594 | 7165 | 708 | CRQ40681.1 | 595 | 7166 | 254 | CRQ40665.1 | 596 | 7167 | 670 |
| 133 | I | 4 | CRQ91532.1 | 598 | 7169 | 731 | CRQ91543.1 | 599 | 7170 | 245 | CRQ91554.1 | 600 | 7171 | 547 |
| 134 | I | 4 | CRQ79081.1 | 602 | 7173 | 708 | CRQ79071.1 | 603 | 7174 | 254 | CRQ79060.1 | 604 | 7175 | 670 |
| 135 | I | 4 | CRR29704.1 | 606 | 7177 | 731 | CRR29682.1 | 607 | 7178 | 245 | CRR29660.1 | 608 | 7179 | 547 |
| 136 | I | 4 | CRR29108.1 | 610 | 7181 | 708 | CRR29090.1 | 611 | 7182 | 254 | CRR29070.1 | 612 | 7183 | 670 |
| 137 | I | 4 | CRR72780.1 | 614 | 7185 | 708 | CRR72789.1 | 615 | 7186 | 254 | CRR72803.1 | 616 | 7187 | 670 |
| 138 | I | 4 | CRR80244.1 | 618 | 7189 | 731 | CRR80233.1 | 619 | 7190 | 245 | CRR80220.1 | 620 | 7191 | 547 |
| 139 | I | 4 | CRR16521.1 | 622 | 7193 | 731 | CRR16537.1 | 623 | 7194 | 245 | CRR16555.1 | 624 | 7195 | 547 |
| 140 | I | 4 | CRR21620.1 | 626 | 7197 | 708 | CRR21592.1 | 627 | 7198 | 254 | CRR21570.1 | 628 | 7199 | 670 |
| 141 | I | 4 | CRQ92468.1 | 630 | 7201 | 731 | CRQ92458.1 | 631 | 7202 | 245 | CRQ92450.1 | 632 | 7203 | 547 |
| 142 | I | 4 | CRQ86065.1 | 634 | 7205 | 708 | CRQ86078.1 | 635 | 7206 | 254 | CRQ86084.1 | 636 | 7207 | 670 |
| 143 | I | 4 | CRQ59609.1 | 638 | 7209 | 731 | CRQ59620.1 | 639 | 7210 | 245 | CRQ59636.1 | 640 | 7211 | 547 |
| 144 | I | 4 | CRQ56245.1 | 642 | 7213 | 708 | CRQ56252.1 | 643 | 7214 | 254 | CRQ56261.1 | 644 | 7215 | 670 |
| 145 | I | 4 | CRR63837.1 | 646 | 7217 | 731 | CRR63852.1 | 647 | 7218 | 245 | CRR63868.1 | 648 | 7219 | 547 |
| 146 | I | 4 | CRR21775.1 | 650 | 7221 | 708 | CRR21754.1 | 651 | 7222 | 254 | CRR21733.1 | 652 | 7223 | 670 |
| 147 | I | 3 | CRO81150.1 | 654 | 7225 | 731 | CRO81176.1 | 655 | 7226 | 245 | CRO81209.1 | 656 | 7227 | 547 |
| 148 | I | 4 | CRP93376.1 | 657 | 7228 | 708 | CRP93350.1 | 658 | 7229 | 254 | CRP93318.1 | 659 | 7230 | 670 |
| 149 | I | 4 | CRR90631.1 | 661 | 7232 | 708 | CRR90638.1 | 662 | 7233 | 254 | CRR90645.1 | 663 | 7234 | 670 |
| 150 | I | 4 | CRS00747.1 | 665 | 7236 | 731 | CRS00755.1 | 666 | 7237 | 245 | CRS00761.1 | 667 | 7238 | 547 |
| 151 | I | 4 | CRR57318.1 | 669 | 7240 | 708 | CRR57304.1 | 670 | 7241 | 254 | CRR57289.1 | 671 | 7242 | 670 |
| 152 | I | 4 | CRR67521.1 | 673 | 7244 | 731 | CRR67533.1 | 674 | 7245 | 245 | CRR67546.1 | 675 | 7246 | 547 |
| 153 | I | 4 | CRR87278.1 | 677 | 7248 | 708 | CRR87267.1 | 678 | 7249 | 254 | CRR87255.1 | 679 | 7250 | 670 |
| 154 | I | 4 | CRS01331.1 | 681 | 7252 | 731 | CRS01339.1 | 682 | 7253 | 245 | CRS01359.1 | 683 | 7254 | 547 |
| 155 | I | 4 | CRX14782.1 | 685 | 7256 | 731 | CRX14787.1 | 686 | 7257 | 245 | CRX14795.1 | 687 | 7258 | 547 |
| 156 | I | 4 | CRX13592.1 | 689 | 7260 | 708 | CRX13600.1 | 690 | 7261 | 254 | CRX13607.1 | 691 | 7262 | 670 |
| 157 | I | 4 | CRX26819.1 | 693 | 7264 | 708 | CRX26816.1 | 694 | 7265 | 254 | CRX26813.1 | 695 | 7266 | 670 |
| 158 | I | 3 | CRX28035.1 | 697 | 7268 | 731 | CRX28038.1 | 698 | 7269 | 245 | CRX28039.1 | 699 | 7270 | 547 |
| 159 | I | 4 | CRP16698.1 | 700 | 7271 | 708 | CRP16667.1 | 701 | 7272 | 254 | CRP16635.1 | 702 | 7273 | 670 |
| 160 | I | 4 | CRP62245.1 | 704 | 7275 | 731 | CRP62208.1 | 705 | 7276 | 245 | CRP62175.1 | 706 | 7277 | 547 |
| 161 | I | 4 | CRO03605.1 | 708 | 7279 | 708 | CRO03569.1 | 709 | 7280 | 254 | CRO03532.1 | 710 | 7281 | 670 |
| 162 | I | 4 | CRO33501.1 | 712 | 7283 | 731 | CRO33459.1 | 713 | 7284 | 245 | CRO33417.1 | 714 | 7285 | 547 |
| 163 | I | 4 | CRS11181.1 | 716 | 7287 | 708 | CRS11171.1 | 717 | 7288 | 254 | CRS11162.1 | 718 | 7289 | 670 |
| 164 | I | 4 | CRS17573.1 | 720 | 7291 | 731 | CRS17567.1 | 721 | 7292 | 245 | CRS17562.1 | 722 | 7293 | 547 |
| 165 | I | 4 | CRR97327.1 | 724 | 7295 | 708 | CRR97346.1 | 725 | 7296 | 254 | CRR97362.1 | 726 | 7297 | 670 |
| 166 | I | 4 | CRS12883.1 | 728 | 7299 | 731 | CRS12896.1 | 729 | 7300 | 245 | CRS12909.1 | 730 | 7301 | 547 |
| 167 | I | 4 | CRP70706.1 | 732 | 7303 | 708 | CRP70732.1 | 733 | 7304 | 254 | CRP70763.1 | 734 | 7305 | 670 |
| 168 | I | 4 | CRO21159.1 | 736 | 7307 | 731 | CRO21133.1 | 737 | 7308 | 245 | CRO21108.1 | 738 | 7309 | 547 |
| 169 | I | 4 | CRO99304.1 | 740 | 7311 | 708 | CRO99327.1 | 741 | 7312 | 254 | CRO99358.1 | 742 | 7313 | 670 |
| 170 | I | 4 | CRP25119.1 | 744 | 7315 | 731 | CRP25093.1 | 745 | 7316 | 245 | CRP25069.1 | 746 | 7317 | 547 |
| 171 | I | 4 | CRP38742.1 | 748 | 7319 | 731 | CRP38712.1 | 749 | 7320 | 245 | CRP38676.1 | 750 | 7321 | 547 |
| 172 | I | 4 | CRP11637.1 | 752 | 7323 | 708 | CRP11607.1 | 753 | 7324 | 254 | CRP11576.1 | 754 | 7325 | 670 |
| 173 | I | 4 | CRP98506.1 | 756 | 7327 | 731 | CRP98537.1 | 757 | 7328 | 245 | CRP98562.1 | 758 | 7329 | 547 |
| 174 | I | 4 | CRP72954.1 | 760 | 7331 | 708 | CRP72921.1 | 761 | 7332 | 254 | CRP72892.1 | 762 | 7333 | 670 |
| 175 | I | 4 | CRX06558.1 | 764 | 7335 | 708 | CRX06566.1 | 765 | 7336 | 254 | CRX06574.1 | 766 | 7337 | 670 |
| 176 | I | 4 | CRX13417.1 | 768 | 7339 | 731 | CRX13424.1 | 769 | 7340 | 245 | CRX13432.1 | 770 | 7341 | 547 |
| 177 | I | 4 | CRR85713.1 | 772 | 7343 | 708 | CRR85702.1 | 773 | 7344 | 254 | CRR85691.1 | 774 | 7345 | 670 |
| 178 | I | 4 | CRR95325.1 | 776 | 7347 | 731 | CRR95318.1 | 777 | 7348 | 245 | CRR95310.1 | 778 | 7349 | 547 |
| 179 | I | 4 | CRR86767.1 | 780 | 7351 | 731 | CRR86753.1 | 781 | 7352 | 245 | CRR86739.1 | 782 | 7353 | 547 |
| 180 | I | 4 | CRR89272.1 | 784 | 7355 | 708 | CRR89280.1 | 785 | 7356 | 254 | CRR89292.1 | 786 | 7357 | 670 |
| 181 | I | 4 | CRS01732.1 | 788 | 7359 | 708 | CRS01741.1 | 789 | 7360 | 254 | CRS01755.1 | 790 | 7361 | 670 |
| 182 | I | 4 | CRS11345.1 | 792 | 7363 | 731 | CRS11336.1 | 793 | 7364 | 245 | CRS11327.1 | 794 | 7365 | 547 |
| 183 | I | 4 | CRR80918.1 | 796 | 7367 | 708 | CRR80905.1 | 797 | 7368 | 254 | CRR80897.1 | 798 | 7369 | 670 |
| 184 | I | 4 | CRR92387.1 | 800 | 7371 | 731 | CRR92379.1 | 801 | 7372 | 245 | CRR92370.1 | 802 | 7373 | 547 |
| 185 | I | 4 | CRR30277.1 | 804 | 7375 | 731 | CRR30230.1 | 805 | 7376 | 245 | CRR30212.1 | 806 | 7377 | 547 |
| 186 | I | 4 | CRS13870.1 | 808 | 7379 | 708 | CRS13874.1 | 809 | 7380 | 254 | CRS13879.1 | 810 | 7381 | 670 |
| 187 | I | 4 | CRS13774.1 | 812 | 7383 | 731 | CRS13765.1 | 813 | 7384 | 245 | CRS13757.1 | 814 | 7385 | 547 |
| 188 | I | 4 | CRR98672.1 | 816 | 7387 | 708 | CRR98685.1 | 817 | 7388 | 254 | CRR98700.1 | 818 | 7389 | 670 |
| 189 | I | 4 | CRS06208.1 | 820 | 7391 | 708 | CRS06197.1 | 821 | 7392 | 254 | CRS06186.1 | 822 | 7393 | 670 |
| 190 | I | 4 | CRS13959.1 | 824 | 7395 | 731 | CRS13968.1 | 825 | 7396 | 245 | CRS13976.1 | 826 | 7397 | 547 |
| 191 | I | 4 | CRW73950.1 | 828 | 7399 | 708 | CRW73964.1 | 829 | 7400 | 254 | CRW73981.1 | 830 | 7401 | 670 |
| 192 | I | 4 | CRX24327.1 | 832 | 7403 | 731 | CRX24332.1 | 833 | 7404 | 245 | CRX24337.1 | 834 | 7405 | 547 |
| 193 | I | 4 | CRX24419.1 | 836 | 7407 | 731 | CRX24414.1 | 837 | 7408 | 245 | CRX24410.1 | 838 | 7409 | 547 |
| 194 | I | 4 | CRX17855.1 | 840 | 7411 | 708 | CRX17846.1 | 841 | 7412 | 254 | CRX17835.1 | 842 | 7413 | 670 |
| 195 | I | 4 | CRR62909.1 | 844 | 7415 | 731 | CRR62897.1 | 845 | 7416 | 245 | CRR62885.1 | 846 | 7417 | 547 |
| 196 | I | 4 | CRX06842.1 | 848 | 7419 | 708 | CRX06835.1 | 849 | 7420 | 254 | CRX06827.1 | 850 | 7421 | 670 |
| 197 | I | 4 | CRX13728.1 | 852 | 7423 | 731 | CRX13735.1 | 853 | 7424 | 245 | CRX13742.1 | 854 | 7425 | 547 |
| 198 | I | 4 | CRR17604.1 | 856 | 7427 | 731 | CRR17580.1 | 857 | 7428 | 245 | CRR17565.1 | 858 | 7429 | 547 |
| 199 | I | 4 | CRQ30116.1 | 860 | 7431 | 708 | CRQ30103.1 | 861 | 7432 | 254 | CRQ30089.1 | 862 | 7433 | 670 |
| 200 | I | 4 | CRR51933.1 | 864 | 7435 | 731 | CRR51916.1 | 865 | 7436 | 245 | CRR51899.1 | 866 | 7437 | 547 |
| 201 | I | 4 | CRQ97183.1 | 868 | 7439 | 708 | CRQ97164.1 | 869 | 7440 | 254 | CRQ97143.1 | 870 | 7441 | 670 |
| 202 | I | 4 | KQJ58852.1 | 872 | 7443 | 708 | KQJ58851.1 | 873 | 7444 | 254 | KQJ58850.1 | 874 | 7445 | 670 |
| 203 | I | 4 | KRU98874.1 | 876 | 7447 | 731 | KRU98873.1 | 877 | 7448 | 245 | KRU98872.1 | 878 | 7449 | 558 |
| 204 | I | 4 | KRV03410.1 | 880 | 7451 | 731 | KRV03411.1 | 881 | 7452 | 245 | KRV03412.1 | 882 | 7453 | 558 |
| 205 | I | 4 | KRU83200.1 | 884 | 7455 | 731 | KRU83199.1 | 885 | 7456 | 245 | KRU83198.1 | 886 | 7457 | 558 |
| 206 | I | 4 | KRU92657.1 | 888 | 7459 | 731 | KRU92656.1 | 889 | 7460 | 245 | KRU92655.1 | 890 | 7461 | 558 |
| 207 | I | 4 | KSD25189.1 | 892 | 7463 | 708 | KSD25190.1 | 893 | 7464 | 254 | KSD25191.1 | 894 | 7465 | 670 |
| 208 | I | 4 | KSD74108.1 | 896 | 7467 | 708 | KSD74107.1 | 897 | 7468 | 254 | KSD74106.1 | 898 | 7469 | 721 |
| 209 | I | 4 | KSD60218.1 | 900 | 7471 | 708 | KSD60219.1 | 901 | 7472 | 254 | KSD60220.1 | 902 | 7473 | 670 |
| 210 | I | 4 | KSD87958.1 | 904 | 7475 | 731 | KSD87957.1 | 905 | 7476 | 245 | KSD87956.1 | 906 | 7477 | 547 |
| 211 | I | 4 | KSD88699.1 | 908 | 7479 | 708 | KSD88700.1 | 909 | 7480 | 254 | KSD88701.1 | 910 | 7481 | 670 |
| 212 | I | 4 | KSD50146.1 | 912 | 7483 | 731 | KSD50145.1 | 913 | 7484 | 245 | KSD50144.1 | 914 | 7485 | 547 |
| 213 | I | 4 | KSD56745.1 | 916 | 7487 | 708 | KSD56744.1 | 917 | 7488 | 254 | KSD56743.1 | 918 | 7489 | 670 |
| 214 | I | 4 | KSE06419.1 | 920 | 7491 | 708 | KSE06418.1 | 921 | 7492 | 254 | KSE06417.1 | 922 | 7493 | 670 |
| 215 | I | 4 | KSD97264.1 | 924 | 7495 | 731 | KSD97265.1 | 925 | 7496 | 245 | KSD97266.1 | 926 | 7497 | 547 |
| 216 | I | 4 | KSE55326.1 | 928 | 7499 | 731 | KSE55325.1 | 929 | 7500 | 245 | KSE55324.1 | 930 | 7501 | 547 |
| 217 | I | 4 | KSE57194.1 | 932 | 7503 | 708 | KSE57195.1 | 933 | 7504 | 254 | KSE57196.1 | 934 | 7505 | 670 |
| 218 | I | 4 | KSE67397.1 | 936 | 7507 | 731 | KSE67396.1 | 937 | 7508 | 245 | KSE67395.1 | 938 | 7509 | 547 |
| 219 | I | 4 | KSE22580.1 | 940 | 7511 | 731 | KSE22579.1 | 941 | 7512 | 245 | KSE22578.1 | 942 | 7513 | 547 |
| 220 | I | 4 | KSE22789.1 | 944 | 7515 | 708 | KSE22790.1 | 945 | 7516 | 254 | KSE22791.1 | 946 | 7517 | 670 |
| 221 | I | 4 | KSG55970.1 | 948 | 7519 | 708 | KSG55971.1 | 949 | 7520 | 254 | KSG55972.1 | 950 | 7521 | 670 |
| 222 | I | 4 | KSH11345.1 | 952 | 7523 | 708 | KSH11346.1 | 953 | 7524 | 254 | KSH11347.1 | 954 | 7525 | 670 |
| 223 | I | 4 | KSC39038.1 | 956 | 7527 | 731 | KSC39037.1 | 957 | 7528 | 245 | KSC39036.1 | 958 | 7529 | 558 |
| 224 | I | 4 | KSC26153.1 | 960 | 7531 | 731 | KSC26154.1 | 961 | 7532 | 245 | KSC26155.1 | 962 | 7533 | 547 |
| 225 | I | 4 | KSC92702.1 | 964 | 7535 | 708 | KSC92703.1 | 965 | 7536 | 254 | KSC92704.1 | 966 | 7537 | 670 |
| 226 | I | 4 | KSD59501.1 | 968 | 7539 | 708 | KSD59502.1 | 969 | 7540 | 254 | KSD59503.1 | 970 | 7541 | 670 |
| 227 | I | 4 | KSD58176.1 | 972 | 7543 | 731 | KSD58175.1 | 973 | 7544 | 245 | KSD58174.1 | 974 | 7545 | 547 |
| 228 | I | 4 | KSE83336.1 | 976 | 7547 | 708 | KSE83337.1 | 977 | 7548 | 254 | KSE83338.1 | 978 | 7549 | 670 |
| 229 | I | 4 | KSI47359.1 | 980 | 7551 | 731 | KSI47360.1 | 981 | 7552 | 245 | KSI47361.1 | 982 | 7553 | 547 |
| 230 | I | 4 | KSI07558.1 | 984 | 7555 | 731 | KSI07557.1 | 985 | 7556 | 245 | KSI07556.1 | 986 | 7557 | 547 |
| 231 | I | 4 | KSJ10824.1 | 988 | 7559 | 708 | KSJ10823.1 | 989 | 7560 | 254 | KSJ10822.1 | 990 | 7561 | 670 |
| 232 | I | 4 | KSJ26235.1 | 992 | 7563 | 708 | KSJ26234.1 | 993 | 7564 | 254 | KSJ26233.1 | 994 | 7565 | 670 |
| 233 | I | 4 | KSJ98813.1 | 996 | 7567 | 731 | KSJ98814.1 | 997 | 7568 | 245 | KSJ98815.1 | 998 | 7569 | 558 |
| 234 | I | 4 | KSK91641.1 | 1000 | 7571 | 731 | KSK91642.1 | 1001 | 7572 | 245 | KSK91643.1 | 1002 | 7573 | 547 |
| 235 | I | 4 | KSL06910.1 | 1004 | 7575 | 731 | KSL06911.1 | 1005 | 7576 | 245 | KSL06912.1 | 1006 | 7577 | 558 |
| 236 | I | 4 | KSM47321.1 | 1008 | 7579 | 731 | KSM47320.1 | 1009 | 7580 | 245 | KSM47319.1 | 1010 | 7581 | 558 |
| 237 | I | 4 | KSM41525.1 | 1012 | 7583 | 708 | KSM41524.1 | 1013 | 7584 | 254 | KSM41523.1 | 1014 | 7585 | 721 |
| 238 | I | 4 | KSN32759.1 | 1016 | 7587 | 731 | KSN32760.1 | 1017 | 7588 | 245 | KSN32761.1 | 1018 | 7589 | 547 |
| 239 | I | 4 | KSN33320.1 | 1020 | 7591 | 708 | KSN33321.1 | 1021 | 7592 | 254 | KSN33322.1 | 1022 | 7593 | 670 |
| 240 | I | 4 | KSN27912.1 | 1024 | 7595 | 731 | KSN27911.1 | 1025 | 7596 | 245 | KSN27910.1 | 1026 | 7597 | 547 |
| 241 | I | 4 | KSN02517.1 | 1028 | 7599 | 708 | KSN02518.1 | 1029 | 7600 | 254 | KSN02519.1 | 1030 | 7601 | 640 |
| 242 | I | 4 | KSN94642.1 | 1032 | 7603 | 731 | KSN94643.1 | 1033 | 7604 | 245 | KSN94644.1 | 1034 | 7605 | 558 |
| 243 | I | 4 | KSN59773.1 | 1036 | 7607 | 708 | KSN59774.1 | 1037 | 7608 | 254 | KSN59775.1 | 1038 | 7609 | 670 |
| 244 | I | 4 | KSN68235.1 | 1040 | 7611 | 731 | KSN68236.1 | 1041 | 7612 | 245 | KSN68237.1 | 1042 | 7613 | 547 |
| 245 | I | 4 | KSO78871.1 | 1044 | 7615 | 731 | KSO78872.1 | 1045 | 7616 | 245 | KSO78873.1 | 1046 | 7617 | 558 |
| 246 | I | 4 | KSO73926.1 | 1048 | 7619 | 731 | KSO73925.1 | 1049 | 7620 | 245 | KSO73924.1 | 1050 | 7621 | 547 |
| 247 | I | 4 | KSO74314.1 | 1052 | 7623 | 708 | KSO74313.1 | 1053 | 7624 | 254 | KSO74312.1 | 1054 | 7625 | 670 |
| 248 | I | 4 | KSO93081.1 | 1056 | 7627 | 708 | KSO93082.1 | 1057 | 7628 | 254 | KSO93083.1 | 1058 | 7629 | 670 |
| 249 | I | 4 | KSO91845.1 | 1060 | 7631 | 731 | KSO91844.1 | 1061 | 7632 | 245 | KSO91843.1 | 1062 | 7633 | 547 |
| 250 | I | 4 | KSP60331.1 | 1064 | 7635 | 708 | KSP60332.1 | 1065 | 7636 | 254 | KSP60333.1 | 1066 | 7637 | 670 |
| 251 | I | 4 | KSP82596.1 | 1068 | 7639 | 782 | KSP82595.1 | 1069 | 7640 | 247 | KSP82594.1 | 1070 | 7641 | 723 |
| 252 | I | 4 | KSQ11856.1 | 1072 | 7643 | 731 | KSQ11855.1 | 1073 | 7644 | 245 | KSQ11854.1 | 1074 | 7645 | 558 |
| 253 | I | 4 | KSN68769.1 | 1076 | 7647 | 731 | KSN68770.1 | 1077 | 7648 | 245 | KSN68771.1 | 1078 | 7649 | 558 |
| 254 | I | 4 | KSO40824.1 | 1080 | 7651 | 708 | KSO40825.1 | 1081 | 7652 | 254 | KSO40826.1 | 1082 | 7653 | 721 |
| 255 | I | 4 | KSQ48400.1 | 1084 | 7655 | 708 | KSQ48399.1 | 1085 | 7656 | 254 | KSQ48398.1 | 1086 | 7657 | 670 |
| 256 | I | 4 | KSQ47084.1 | 1088 | 7659 | 731 | KSQ47083.1 | 1089 | 7660 | 245 | KSQ47082.1 | 1090 | 7661 | 547 |
| 257 | I | 4 | KSQ03908.1 | 1092 | 7663 | 708 | KSQ03907.1 | 1093 | 7664 | 254 | KSQ03906.1 | 1094 | 7665 | 670 |
| 258 | I | 4 | KSQ01968.1 | 1096 | 7667 | 731 | KSQ01967.1 | 1097 | 7668 | 245 | KSQ01966.1 | 1098 | 7669 | 547 |
| 259 | I | 4 | KSR61594.1 | 1100 | 7671 | 708 | KSR61593.1 | 1101 | 7672 | 254 | KSR61592.1 | 1102 | 7673 | 721 |
| 260 | I | 4 | KSR90896.1 | 1104 | 7675 | 708 | KSR90897.1 | 1105 | 7676 | 254 | KSR90898.1 | 1106 | 7677 | 670 |
| 261 | I | 4 | KSR87673.1 | 1108 | 7679 | 731 | KSR87674.1 | 1109 | 7680 | 245 | KSR87675.1 | 1110 | 7681 | 558 |
| 262 | I | 4 | KSR56106.1 | 1112 | 7683 | 731 | KSR56105.1 | 1113 | 7684 | 245 | KSR56104.1 | 1114 | 7685 | 547 |
| 263 | I | 4 | KSR68843.1 | 1116 | 7687 | 708 | KSR68842.1 | 1117 | 7688 | 254 | KSR68841.1 | 1118 | 7689 | 670 |
| 264 | I | 4 | KSS83648.1 | 1120 | 7691 | 708 | KSS83647.1 | 1121 | 7692 | 254 | KSS83646.1 | 1122 | 7693 | 670 |
| 265 | I | 4 | KSS82297.1 | 1124 | 7695 | 731 | KSS82296.1 | 1125 | 7696 | 245 | KSS82295.1 | 1126 | 7697 | 547 |
| 266 | I | 4 | KSS65852.1 | 1128 | 7699 | 731 | KSS65851.1 | 1129 | 7700 | 245 | KSS65850.1 | 1130 | 7701 | 547 |
| 267 | I | 4 | KSS71074.1 | 1132 | 7703 | 708 | KSS71073.1 | 1133 | 7704 | 254 | KSS71072.1 | 1134 | 7705 | 670 |
| 268 | I | 4 | KSS83671.1 | 1136 | 7707 | 731 | KSS83670.1 | 1137 | 7708 | 245 | KSS83669.1 | 1138 | 7709 | 547 |
| 269 | I | 4 | KSS97189.1 | 1140 | 7711 | 708 | KSS97188.1 | 1141 | 7712 | 254 | KSS97187.1 | 1142 | 7713 | 670 |
| 270 | I | 4 | KSS68542.1 | 1144 | 7715 | 731 | KSS68541.1 | 1145 | 7716 | 245 | KSS68540.1 | 1146 | 7717 | 547 |
| 271 | I | 4 | KSS64139.1 | 1148 | 7719 | 708 | KSS64138.1 | 1149 | 7720 | 254 | KSS64137.1 | 1150 | 7721 | 670 |
| 272 | I | 4 | KST02401.1 | 1152 | 7723 | 708 | KST02402.1 | 1153 | 7724 | 254 | KST02403.1 | 1154 | 7725 | 670 |
| 273 | I | 4 | KST01506.1 | 1156 | 7727 | 731 | KST01505.1 | 1157 | 7728 | 245 | KST01504.1 | 1158 | 7729 | 547 |
| 274 | I | 4 | KST09053.1 | 1160 | 7731 | 708 | KST09052.1 | 1161 | 7732 | 254 | KST09051.1 | 1162 | 7733 | 670 |
| 275 | I | 4 | KST07356.1 | 1164 | 7735 | 731 | KST07355.1 | 1165 | 7736 | 245 | KST07354.1 | 1166 | 7737 | 547 |
| 276 | I | 4 | KSR39888.1 | 1168 | 7739 | 708 | KSR39889.1 | 1169 | 7740 | 254 | KSR39890.1 | 1170 | 7741 | 670 |
| 277 | I | 4 | KST03849.1 | 1172 | 7743 | 731 | KST03848.1 | 1173 | 7744 | 245 | KST03847.1 | 1174 | 7745 | 547 |
| 278 | I | 4 | KST05251.1 | 1176 | 7747 | 708 | KST05250.1 | 1177 | 7748 | 254 | KST05249.1 | 1178 | 7749 | 670 |
| 279 | I | 4 | KSS85815.1 | 1180 | 7751 | 708 | KSS85816.1 | 1181 | 7752 | 254 | KSS85817.1 | 1182 | 7753 | 670 |
| 280 | I | 4 | KSS83938.1 | 1184 | 7755 | 731 | KSS83937.1 | 1185 | 7756 | 245 | KSS83936.1 | 1186 | 7757 | 547 |
| 281 | I | 4 | KTF54396.1 | 1188 | 7759 | 708 | KTF54397.1 | 1189 | 7760 | 254 | KTF54398.1 | 1190 | 7761 | 670 |
| 282 | I | 4 | KTF49811.1 | 1192 | 7763 | 731 | KTF49810.1 | 1193 | 7764 | 245 | KTF49809.1 | 1194 | 7765 | 547 |
| 283 | I | 4 | KTK55766.1 | 1196 | 7767 | 782 | KTK55767.1 | 1197 | 7768 | 247 | KTK55768.1 | 1198 | 7769 | 723 |
| 284 | I | 4 | ALZ13984.1 | 1200 | 7771 | 782 | ALZ13983.1 | 1201 | 7772 | 247 | ALZ13982.1 | 1202 | 7773 | 723 |
| 285 | I | 4 | ALY33856.1 | 1204 | 7775 | 731 | ALY33855.1 | 1205 | 7776 | 245 | ALY33854.1 | 1206 | 7777 | 558 |
| 286 | I | 4 | ALY39460.1 | 1208 | 7779 | 731 | ALY39461.1 | 1209 | 7780 | 245 | ALY39462.1 | 1210 | 7781 | 547 |
| 287 | I | 4 | ALY43157.1 | 1212 | 7783 | 708 | ALY43156.1 | 1213 | 7784 | 254 | ALY43155.1 | 1214 | 7785 | 670 |
| 288 | I | 3 | KWR99692.1 | 1216 | 7787 | 731 | KWR99691.1 | 1217 | 7788 | 245 | KWR99690.1 | 1218 | 7789 | 547 |
| 289 | I | 3 | KWZ69450.1 | 1219 | 7790 | 708 | KWZ69449.1 | 1220 | 7791 | 254 | | | | |
| 290 | I | 4 | KXG16565.1 | 1222 | 7793 | 708 | KXG16566.1 | 1223 | 7794 | 254 | KXG16567.1 | 1224 | 7795 | 670 |
| 291 | I | 4 | KYO75360.1 | 1226 | 7797 | 708 | KYO75361.1 | 1227 | 7798 | 254 | KYO75362.1 | 1228 | 7799 | 670 |
| 292 | I | 3 | KIS47213.1 | 1230 | 7801 | 670 | KIS48415.1 | 1231 | 7802 | 131 | | | | |
| 293 | I | 4 | KVA56588.1 | 1233 | 7804 | 670 | KVA56589.1 | 1234 | 7805 | 241 | KVA56590.1 | 1235 | 7806 | 545 |
| 294 | I | 4 | KVC33765.1 | 1237 | 7808 | 670 | KVC33766.1 | 1238 | 7809 | 241 | KVC33767.1 | 1239 | 7810 | 545 |
| 295 | I | 4 | AEV65174.1 | 1241 | 7812 | 608 | AEV65173.1 | 1242 | 7813 | 247 | AEV65172.1 | 1243 | 7814 | 542 |
| 296 | I | 4 | KGE64068.1 | 1245 | 7816 | 730 | KGE64069.1 | 1246 | 7817 | 247 | KGE64070.1 | 1247 | 7818 | 542 |
| 297 | I | 4 | KWV80506.1 | 1249 | 7820 | 781 | KWV80505.1 | 1250 | 7821 | 248 | KWV80504.1 | 1251 | 7822 | 704 |
| 298 | I | 4 | ABQ80738.1 | 1253 | 7824 | 730 | ABQ80737.1 | 1254 | 7825 | 247 | ABQ80736.1 | 1255 | 7826 | 541 |
| 299 | I | 4 | AFK68697.1 | 1257 | 7828 | 730 | AFK68696.1 | 1258 | 7829 | 247 | AFK68695.1 | 1259 | 7830 | 541 |
| 300 | I | 4 | EMR48530.1 | 1261 | 7832 | 781 | EMR48531.1 | 1262 | 7833 | 254 | EMR48532.1 | 1263 | 7834 | 681 |
| 301 | I | 4 | AGA73017.1 | 1265 | 7836 | 730 | AGA73018.1 | 1266 | 7837 | 247 | AGA73019.1 | 1267 | 7838 | 542 |
| 302 | I | 4 | KTC24575.1 | 1269 | 7840 | 782 | KTC24464.1 | 1270 | 7841 | 247 | KTC24463.1 | 1271 | 7842 | 626 |
| 303 | I | 4 | ABP79163.1 | 1273 | 7844 | 783 | ABP79164.1 | 1274 | 7845 | 254 | ABP79165.1 | 1275 | 7846 | 210 |
| 304 | I | 4 | AEA83397.1 | 1277 | 7848 | 781 | AEA83398.1 | 1278 | 7849 | 254 | AEA83399.1 | 1279 | 7850 | 751 |
| 305 | I | 4 | EHY79730.1 | 1281 | 7852 | 781 | EHY79731.1 | 1282 | 7853 | 254 | EHY79732.1 | 1283 | 7854 | 751 |
| 306 | I | 4 | AFM32806.1 | 1285 | 7856 | 781 | AFM32807.1 | 1286 | 7857 | 254 | AFM32808.1 | 1287 | 7858 | 752 |
| 307 | I | 4 | EWC39447.1 | 1289 | 7860 | 682 | EWC39446.1 | 1290 | 7861 | 245 | EWC39445.1 | 1291 | 7862 | 558 |
| 308 | I | 3 | EPL62183.1 | 1293 | 7864 | 781 | EPL62184.1 | 1294 | 7865 | 254 | | | | |
| 309 | I | 4 | EPM44374.1 | 1296 | 7867 | 659 | EPM44375.1 | 1297 | 7868 | 247 | EPM44377.1 | 1298 | 7869 | 378 |
| 310 | I | 4 | EPN29003.1 | 1300 | 7871 | 659 | EPN29004.1 | 1301 | 7872 | 247 | EPN29005.1 | 1302 | 7873 | 518 |
| 311 | I | 4 | EPN47277.1 | 1304 | 7875 | 659 | EPN47278.1 | 1305 | 7876 | 247 | EPN47280.1 | 1306 | 7877 | 378 |
| 312 | I | 4 | EPN38583.1 | 1308 | 7879 | 659 | EPN38584.1 | 1309 | 7880 | 247 | EPN38585.1 | 1310 | 7881 | 545 |
| 313 | I | 4 | EPN31613.1 | 1312 | 7883 | 659 | EPN31614.1 | 1313 | 7884 | 247 | EPN31616.1 | 1314 | 7885 | 378 |
| 314 | I | 4 | EPN22775.1 | 1316 | 7887 | 659 | EPN22776.1 | 1317 | 7888 | 247 | EPN22777.1 | 1318 | 7889 | 545 |
| 315 | I | 4 | EPN13939.1 | 1320 | 7891 | 659 | EPN13938.1 | 1321 | 7892 | 247 | EPN13936.1 | 1322 | 7893 | 378 |
| 316 | I | 4 | EPM64842.1 | 1324 | 7895 | 659 | EPM64843.1 | 1325 | 7896 | 247 | EPM64844.1 | 1326 | 7897 | 545 |
| 317 | I | 4 | KFE44523.1 | 1328 | 7899 | 730 | KFE44522.1 | 1329 | 7900 | 247 | KFE44521.1 | 1330 | 7901 | 542 |
| 318 | I | 4 | KTC49637.1 | 1332 | 7903 | 730 | KTC49636.1 | 1333 | 7904 | 247 | KTC49752.1 | 1334 | 7905 | 545 |
| 319 | I | 4 | AAM40747.1 | 1336 | 7907 | 629 | AAM40748.1 | 1337 | 7908 | 238 | AAM40749.1 | 1338 | 7909 | 491 |
| 320 | I | 4 | AAY49837.1 | 1340 | 7911 | 629 | AAY49836.1 | 1341 | 7912 | 238 | AAY49835.1 | 1342 | 7913 | 491 |
| 321 | I | 4 | AKS16757.1 | 1344 | 7915 | 629 | AKS16756.1 | 1345 | 7916 | 238 | AKS18353.1 | 1346 | 7917 | 491 |
| 322 | I | 4 | ACP24372.1 | 1348 | 7919 | 481 | ACP24373.1 | 1349 | 7920 | 309 | ACP24374.1 | 1350 | 7921 | 437 |
| 323 | I | 3 | KUM25652.1 | 1352 | 7923 | 675 | KUM25651.1 | 1353 | 7924 | 323 | KUM25650.1 | 1354 | 7925 | 413 |
| 324 | I | 4 | KUM27288.1 | 1355 | 7926 | 723 | KUM27287.1 | 1356 | 7927 | 302 | KUM27286.1 | 1357 | 7928 | 460 |
| 325 | I | 4 | CDZ72588.1 | 1359 | 7930 | 677 | CDZ72587.1 | 1360 | 7931 | 326 | CDZ72586.1 | 1361 | 7932 | 389 |
| 326 | I | 4 | ACK84139.1 | 1363 | 7934 | 685 | ACK84140.1 | 1364 | 7935 | 296 | ACK84141.1 | 1365 | 7936 | 438 |
| 327 | I | 4 | CAH15308.1 | 1367 | 7938 | 657 | CAH15307.1 | 1368 | 7939 | 234 | CAH15306.1 | 1369 | 7940 | 482 |
| 328 | I | 4 | CCD05132.1 | 1371 | 7942 | 636 | CCD05131.1 | 1372 | 7943 | 234 | CCD05130.1 | 1373 | 7944 | 482 |
| 329 | I | 4 | CZL99121.1 | 1375 | 7946 | 636 | CZL99087.1 | 1376 | 7947 | 234 | CZL99058.1 | 1377 | 7948 | 482 |
| 330 | I | 4 | CZN28597.1 | 1379 | 7950 | 636 | CZN28565.1 | 1380 | 7951 | 234 | CZN28532.1 | 1381 | 7952 | 482 |
| 331 | I | 4 | CZP40404.1 | 1383 | 7954 | 636 | CZP40292.1 | 1384 | 7955 | 234 | CZP40266.1 | 1385 | 7956 | 482 |
| 332 | I | 4 | CZP31110.1 | 1387 | 7958 | 636 | CZP31151.1 | 1388 | 7959 | 234 | CZP31189.1 | 1389 | 7960 | 482 |
| 333 | I | 4 | CZP19884.1 | 1391 | 7962 | 636 | CZP19847.1 | 1392 | 7963 | 234 | CZP19810.1 | 1393 | 7964 | 482 |
| 334 | I | 4 | CZN56655.1 | 1395 | 7966 | 636 | CZN56617.1 | 1396 | 7967 | 234 | CZN56586.1 | 1397 | 7968 | 482 |
| 335 | I | 4 | CZN22035.1 | 1399 | 7970 | 636 | CZN21995.1 | 1400 | 7971 | 234 | CZN21949.1 | 1401 | 7972 | 482 |
| 336 | I | 4 | CZP49453.1 | 1403 | 7974 | 636 | CZP49417.1 | 1404 | 7975 | 234 | CZP49379.1 | 1405 | 7976 | 482 |
| 337 | I | 4 | CZP60974.1 | 1407 | 7978 | 636 | CZP61042.1 | 1408 | 7979 | 234 | CZP61132.1 | 1409 | 7980 | 482 |
| 338 | I | 4 | CZN58239.1 | 1411 | 7982 | 636 | CZN58276.1 | 1412 | 7983 | 234 | CZN58324.1 | 1413 | 7984 | 482 |
| 339 | I | 4 | CZO62381.1 | 1415 | 7986 | 636 | CZO62416.1 | 1416 | 7987 | 234 | CZO62452.1 | 1417 | 7988 | 482 |
| 340 | I | 4 | CZO85409.1 | 1419 | 7990 | 636 | CZO85444.1 | 1420 | 7991 | 234 | CZO85483.1 | 1421 | 7992 | 482 |
| 341 | I | 4 | CZN18702.1 | 1423 | 7994 | 636 | CZN18741.1 | 1424 | 7995 | 234 | CZN18776.1 | 1425 | 7996 | 482 |
| 342 | I | 4 | CZO33191.1 | 1427 | 7998 | 636 | CZO33228.1 | 1428 | 7999 | 234 | CZO33267.1 | 1429 | 8000 | 482 |
| 343 | I | 4 | CZN48685.1 | 1431 | 8002 | 636 | CZN48726.1 | 1432 | 8003 | 234 | CZN48784.1 | 1433 | 8004 | 482 |
| 344 | I | 4 | CZO35301.1 | 1435 | 8006 | 636 | CZO35341.1 | 1436 | 8007 | 234 | CZO35376.1 | 1437 | 8008 | 482 |
| 345 | I | 4 | CZL93468.1 | 1439 | 8010 | 636 | CZL93499.1 | 1440 | 8011 | 234 | CZL93535.1 | 1441 | 8012 | 482 |
| 346 | I | 4 | CZH31955.1 | 1443 | 8014 | 636 | CZH31923.1 | 1444 | 8015 | 234 | CZH31901.1 | 1445 | 8016 | 482 |
| 347 | I | 4 | CZG09013.1 | 1447 | 8018 | 636 | CZG09061.1 | 1448 | 8019 | 234 | CZG09117.1 | 1449 | 8020 | 482 |
| 348 | I | 4 | CZO58196.1 | 1451 | 8022 | 636 | CZO58161.1 | 1452 | 8023 | 234 | CZO58116.1 | 1453 | 8024 | 482 |
| 349 | I | 4 | CZP31281.1 | 1455 | 8026 | 636 | CZP31240.1 | 1456 | 8027 | 234 | CZP31204.1 | 1457 | 8028 | 482 |
| 350 | I | 4 | CZP25054.1 | 1459 | 8030 | 636 | CZP25088.1 | 1460 | 8031 | 234 | CZP25128.1 | 1461 | 8032 | 482 |
| 351 | I | 4 | CZO87032.1 | 1463 | 8034 | 636 | CZO87064.1 | 1464 | 8035 | 234 | CZO87079.1 | 1465 | 8036 | 482 |
| 352 | I | 4 | CZP12610.1 | 1467 | 8038 | 636 | CZP12634.1 | 1468 | 8039 | 234 | CZP12658.1 | 1469 | 8040 | 482 |
| 353 | I | 4 | CZO76824.1 | 1471 | 8042 | 636 | CZO76768.1 | 1472 | 8043 | 234 | CZO76725.1 | 1473 | 8044 | 482 |
| 354 | I | 4 | CZM76075.1 | 1475 | 8046 | 636 | CZM76043.1 | 1476 | 8047 | 234 | CZM76014.1 | 1477 | 8048 | 482 |
| 355 | I | 4 | CZM80398.1 | 1479 | 8050 | 636 | CZM80434.1 | 1480 | 8051 | 234 | CZM80464.1 | 1481 | 8052 | 482 |
| 356 | I | 4 | CZO03095.1 | 1483 | 8054 | 636 | CZO03125.1 | 1484 | 8055 | 234 | CZO03159.1 | 1485 | 8056 | 482 |
| 357 | I | 4 | CZO99703.1 | 1487 | 8058 | 636 | CZO99672.1 | 1488 | 8059 | 234 | CZO99644.1 | 1489 | 8060 | 482 |
| 358 | I | 4 | CZN78011.1 | 1491 | 8062 | 636 | CZN77981.1 | 1492 | 8063 | 234 | CZN77962.1 | 1493 | 8064 | 482 |
| 359 | I | 4 | CZO69823.1 | 1495 | 8066 | 636 | CZO69863.1 | 1496 | 8067 | 234 | CZO69902.1 | 1497 | 8068 | 482 |
| 360 | I | 4 | CZO98643.1 | 1499 | 8070 | 636 | CZO98681.1 | 1500 | 8071 | 234 | CZO98721.1 | 1501 | 8072 | 482 |
| 361 | I | 4 | CZN87164.1 | 1503 | 8074 | 636 | CZN87203.1 | 1504 | 8075 | 234 | CZN87237.1 | 1505 | 8076 | 482 |
| 362 | I | 4 | CZO29078.1 | 1507 | 8078 | 636 | CZO29056.1 | 1508 | 8079 | 234 | CZO29002.1 | 1509 | 8080 | 482 |
| 363 | I | 4 | CZO58157.1 | 1511 | 8082 | 636 | CZO58193.1 | 1512 | 8083 | 234 | CZO58228.1 | 1513 | 8084 | 482 |
| 364 | I | 4 | CZM99522.1 | 1515 | 8086 | 636 | CZM99497.1 | 1516 | 8087 | 234 | CZM99482.1 | 1517 | 8088 | 482 |
| 365 | I | 4 | CZN97533.1 | 1519 | 8090 | 636 | CZN97560.1 | 1520 | 8091 | 234 | CZN97585.1 | 1521 | 8092 | 482 |
| 366 | I | 4 | CZN56114.1 | 1523 | 8094 | 636 | CZN56138.1 | 1524 | 8095 | 234 | CZN56241.1 | 1525 | 8096 | 482 |
| 367 | I | 4 | CZP07429.1 | 1527 | 8098 | 636 | CZP07390.1 | 1528 | 8099 | 234 | CZP07358.1 | 1529 | 8100 | 482 |
| 368 | I | 4 | CZP63511.1 | 1531 | 8102 | 636 | CZP63546.1 | 1532 | 8103 | 234 | CZP63581.1 | 1533 | 8104 | 482 |
| 369 | I | 4 | CZM58627.1 | 1535 | 8106 | 636 | CZM58593.1 | 1536 | 8107 | 234 | CZM58562.1 | 1537 | 8108 | 482 |
| 370 | I | 4 | CZN95768.1 | 1539 | 8110 | 636 | CZN95715.1 | 1540 | 8111 | 234 | CZN95664.1 | 1541 | 8112 | 482 |
| 371 | I | 4 | CZN53092.1 | 1543 | 8114 | 636 | CZN53126.1 | 1544 | 8115 | 234 | CZN53175.1 | 1545 | 8116 | 482 |
| 372 | I | 4 | CZN21729.1 | 1547 | 8118 | 636 | CZN21768.1 | 1548 | 8119 | 234 | CZN21791.1 | 1549 | 8120 | 482 |
| 373 | I | 4 | CZO54055.1 | 1551 | 8122 | 636 | CZO54008.1 | 1552 | 8123 | 234 | CZO53982.1 | 1553 | 8124 | 482 |
| 374 | I | 4 | CZO83037.1 | 1555 | 8126 | 636 | CZO83063.1 | 1556 | 8127 | 234 | CZO83078.1 | 1557 | 8128 | 482 |
| 375 | I | 4 | CZM80662.1 | 1559 | 8130 | 636 | CZM80631.1 | 1560 | 8131 | 234 | CZM80598.1 | 1561 | 8132 | 482 |
| 376 | I | 4 | CZN76327.1 | 1563 | 8134 | 636 | CZN76305.1 | 1564 | 8135 | 234 | CZN76257.1 | 1565 | 8136 | 482 |
| 377 | I | 4 | CZN17181.1 | 1567 | 8138 | 636 | CZN17217.1 | 1568 | 8139 | 234 | CZN17250.1 | 1569 | 8140 | 482 |
| 378 | I | 4 | CZN08820.1 | 1571 | 8142 | 636 | CZN08778.1 | 1572 | 8143 | 234 | CZN08740.1 | 1573 | 8144 | 482 |
| 379 | I | 4 | CZN07776.1 | 1575 | 8146 | 636 | CZN07744.1 | 1576 | 8147 | 234 | CZN07717.1 | 1577 | 8148 | 482 |
| 380 | I | 4 | CZN26973.1 | 1579 | 8150 | 636 | CZN27020.1 | 1580 | 8151 | 234 | CZN27059.1 | 1581 | 8152 | 482 |
| 381 | I | 4 | CZO91605.1 | 1583 | 8154 | 636 | CZO91570.1 | 1584 | 8155 | 234 | CZO91532.1 | 1585 | 8156 | 482 |
| 382 | I | 4 | CZN16140.1 | 1587 | 8158 | 636 | CZN16106.1 | 1588 | 8159 | 234 | CZN16089.1 | 1589 | 8160 | 482 |
| 383 | I | 4 | CZN39081.1 | 1591 | 8162 | 636 | CZN39046.1 | 1592 | 8163 | 234 | CZN38988.1 | 1593 | 8164 | 482 |
| 384 | I | 4 | CZI18710.1 | 1595 | 8166 | 636 | CZI18679.1 | 1596 | 8167 | 234 | CZI18647.1 | 1597 | 8168 | 482 |
| 385 | I | 4 | CZL86382.1 | 1599 | 8170 | 636 | CZL86343.1 | 1600 | 8171 | 234 | CZL86298.1 | 1601 | 8172 | 482 |
| 386 | I | 4 | CZM41589.1 | 1603 | 8174 | 636 | CZM41618.1 | 1604 | 8175 | 234 | CZM41656.1 | 1605 | 8176 | 482 |
| 387 | I | 4 | CZM78595.1 | 1607 | 8178 | 636 | CZM78547.1 | 1608 | 8179 | 234 | CZM78507.1 | 1609 | 8180 | 482 |
| 388 | I | 4 | CZM56824.1 | 1611 | 8182 | 636 | CZM56782.1 | 1612 | 8183 | 234 | CZM56749.1 | 1613 | 8184 | 482 |
| 389 | I | 4 | CZM61973.1 | 1615 | 8186 | 636 | CZM62010.1 | 1616 | 8187 | 234 | CZM62034.1 | 1617 | 8188 | 482 |
| 390 | I | 4 | CZM06139.1 | 1619 | 8190 | 636 | CZM06106.1 | 1620 | 8191 | 234 | CZM06061.1 | 1621 | 8192 | 482 |
| 391 | I | 4 | CZM97949.1 | 1623 | 8194 | 636 | CZM97974.1 | 1624 | 8195 | 234 | CZM97996.1 | 1625 | 8196 | 482 |
| 392 | I | 4 | CZN32008.1 | 1627 | 8198 | 636 | CZN32037.1 | 1628 | 8199 | 234 | CZN32065.1 | 1629 | 8200 | 482 |
| 393 | I | 4 | CZN27707.1 | 1631 | 8202 | 636 | CZN27733.1 | 1632 | 8203 | 234 | CZN27771.1 | 1633 | 8204 | 482 |
| 394 | I | 4 | CZM96765.1 | 1635 | 8206 | 636 | CZM96741.1 | 1636 | 8207 | 234 | CZM96707.1 | 1637 | 8208 | 482 |
| 395 | I | 4 | CZN17300.1 | 1639 | 8210 | 636 | CZN17329.1 | 1640 | 8211 | 234 | CZN17356.1 | 1641 | 8212 | 482 |
| 396 | I | 4 | CZN17894.1 | 1643 | 8214 | 636 | CZN17862.1 | 1644 | 8215 | 234 | CZN17835.1 | 1645 | 8216 | 482 |
| 397 | I | 4 | CZN51335.1 | 1647 | 8218 | 636 | CZN51299.1 | 1648 | 8219 | 234 | CZN51263.1 | 1649 | 8220 | 482 |
| 398 | I | 4 | CZN80164.1 | 1651 | 8222 | 636 | CZN80114.1 | 1652 | 8223 | 234 | CZN80081.1 | 1653 | 8224 | 482 |
| 399 | I | 4 | CZN83467.1 | 1655 | 8226 | 636 | CZN83507.1 | 1656 | 8227 | 234 | CZN83551.1 | 1657 | 8228 | 482 |
| 400 | I | 4 | CZN87990.1 | 1659 | 8230 | 636 | CZN87946.1 | 1660 | 8231 | 234 | CZN87903.1 | 1661 | 8232 | 482 |
| 401 | I | 4 | CZN93946.1 | 1663 | 8234 | 636 | CZN93993.1 | 1664 | 8235 | 234 | CZN94035.1 | 1665 | 8236 | 482 |
| 402 | I | 4 | CZO07075.1 | 1667 | 8238 | 636 | CZO07110.1 | 1668 | 8239 | 234 | CZO07154.1 | 1669 | 8240 | 482 |
| 403 | I | 4 | CZO12027.1 | 1671 | 8242 | 636 | CZO11987.1 | 1672 | 8243 | 234 | CZO11952.1 | 1673 | 8244 | 482 |
| 404 | I | 4 | CZO09452.1 | 1675 | 8246 | 636 | CZO09406.1 | 1676 | 8247 | 234 | CZO09365.1 | 1677 | 8248 | 482 |
| 405 | I | 4 | CZN88093.1 | 1679 | 8250 | 636 | CZN88057.1 | 1680 | 8251 | 234 | CZN88014.1 | 1681 | 8252 | 482 |
| 406 | I | 4 | CZO32095.1 | 1683 | 8254 | 636 | CZO32135.1 | 1684 | 8255 | 234 | CZO32185.1 | 1685 | 8256 | 482 |
| 407 | I | 4 | CZO07325.1 | 1687 | 8258 | 636 | CZO07290.1 | 1688 | 8259 | 234 | CZO07266.1 | 1689 | 8260 | 482 |
| 408 | I | 4 | CZO04199.1 | 1691 | 8262 | 636 | CZO04172.1 | 1692 | 8263 | 234 | CZO04137.1 | 1693 | 8264 | 482 |
| 409 | I | 4 | CZN90628.1 | 1695 | 8266 | 636 | CZN90659.1 | 1696 | 8267 | 234 | CZN90697.1 | 1697 | 8268 | 482 |
| 410 | I | 4 | CZP18446.1 | 1699 | 8270 | 636 | CZP18413.1 | 1700 | 8271 | 234 | CZP18376.1 | 1701 | 8272 | 482 |
| 411 | I | 4 | CZP26391.1 | 1703 | 8274 | 636 | CZP26289.1 | 1704 | 8275 | 234 | CZP26260.1 | 1705 | 8276 | 482 |
| 412 | I | 4 | CZP25516.1 | 1707 | 8278 | 636 | CZP25483.1 | 1708 | 8279 | 234 | CZP25450.1 | 1709 | 8280 | 482 |
| 413 | I | 4 | CZN32104.1 | 1711 | 8282 | 636 | CZN32073.1 | 1712 | 8283 | 234 | CZN32054.1 | 1713 | 8284 | 482 |
| 414 | I | 4 | CZO49179.1 | 1715 | 8286 | 636 | CZO49222.1 | 1716 | 8287 | 234 | CZO49269.1 | 1717 | 8288 | 482 |
| 415 | I | 4 | CZO32890.1 | 1719 | 8290 | 636 | CZO32855.1 | 1720 | 8291 | 234 | CZO32827.1 | 1721 | 8292 | 482 |
| 416 | I | 4 | CZO62310.1 | 1723 | 8294 | 636 | CZO62353.1 | 1724 | 8295 | 234 | CZO62389.1 | 1725 | 8296 | 482 |
| 417 | I | 4 | CZO58294.1 | 1727 | 8298 | 636 | CZO58318.1 | 1728 | 8299 | 234 | CZO58349.1 | 1729 | 8300 | 482 |
| 418 | I | 4 | CZO85873.1 | 1731 | 8302 | 636 | CZO85828.1 | 1732 | 8303 | 234 | CZO85797.1 | 1733 | 8304 | 482 |
| 419 | I | 4 | CZP51088.1 | 1735 | 8306 | 636 | CZP51125.1 | 1736 | 8307 | 234 | CZP51157.1 | 1737 | 8308 | 482 |
| 420 | I | 4 | CZO57150.1 | 1739 | 8310 | 636 | CZO57187.1 | 1740 | 8311 | 234 | CZO57213.1 | 1741 | 8312 | 482 |
| 421 | I | 4 | CZO45304.1 | 1743 | 8314 | 636 | CZO45268.1 | 1744 | 8315 | 234 | CZO45218.1 | 1745 | 8316 | 482 |
| 422 | I | 4 | CZO91199.1 | 1747 | 8318 | 636 | CZO91233.1 | 1748 | 8319 | 234 | CZO91266.1 | 1749 | 8320 | 482 |
| 423 | I | 4 | CZN53802.1 | 1751 | 8322 | 636 | CZN53843.1 | 1752 | 8323 | 234 | CZN53877.1 | 1753 | 8324 | 482 |
| 424 | I | 4 | CZN51375.1 | 1755 | 8326 | 636 | CZN51423.1 | 1756 | 8327 | 234 | CZN51467.1 | 1757 | 8328 | 482 |
| 425 | I | 4 | CZO76010.1 | 1759 | 8330 | 636 | CZO76045.1 | 1760 | 8331 | 234 | CZO76083.1 | 1761 | 8332 | 482 |
| 426 | I | 4 | CZR30116.1 | 1763 | 8334 | 636 | CZR30113.1 | 1764 | 8335 | 234 | CZR30109.1 | 1765 | 8336 | 482 |
| 427 | I | 4 | KTD18860.1 | 1767 | 8338 | 634 | KTD18861.1 | 1768 | 8339 | 234 | KTD18862.1 | 1769 | 8340 | 482 |
| 428 | I | 3 | EMU26880.1 | 1771 | 8342 | 686 | EMU26879.1 | 1772 | 8343 | 243 | EMU26878.1 | 1773 | 8344 | 456 |
| 429 | I | 3 | EMT95386.1 | 1774 | 8345 | 686 | EMT95385.1 | 1775 | 8346 | 243 | EMT95384.1 | 1776 | 8347 | 456 |
| 430 | I | 4 | EMT94539.1 | 1777 | 8348 | 686 | EMT94538.1 | 1778 | 8349 | 243 | EMT94537.1 | 1779 | 8350 | 456 |
| 431 | I | 3 | EMU11117.1 | 1781 | 8352 | 686 | EMU11116.1 | 1782 | 8353 | 243 | EMU11115.1 | 1783 | 8354 | 387 |
| 432 | I | 4 | EMU02182.1 | 1784 | 8355 | 686 | EMU02181.1 | 1785 | 8356 | 243 | EMU02180.1 | 1786 | 8357 | 456 |
| 433 | I | 4 | EXB30082.1 | 1788 | 8359 | 686 | EXB30083.1 | 1789 | 8360 | 243 | EXB30084.1 | 1790 | 8361 | 466 |
| 434 | I | 4 | EXB35593.1 | 1792 | 8363 | 686 | EXB35594.1 | 1793 | 8364 | 243 | EXB35595.1 | 1794 | 8365 | 466 |
| 435 | I | 4 | EXB05632.1 | 1796 | 8367 | 686 | EXB05631.1 | 1797 | 8368 | 243 | EXB05630.1 | 1798 | 8369 | 466 |
| 436 | I | 4 | EXB92756.1 | 1800 | 8371 | 686 | EXB92873.1 | 1801 | 8372 | 243 | EXB92651.1 | 1802 | 8373 | 466 |
| 437 | I | 4 | EXB73471.1 | 1804 | 8375 | 686 | EXB73472.1 | 1805 | 8376 | 243 | EXB73473.1 | 1806 | 8377 | 466 |
| 438 | I | 4 | EXE59934.1 | 1808 | 8379 | 686 | EXE59933.1 | 1809 | 8380 | 243 | EXE59932.1 | 1810 | 8381 | 471 |
| 439 | I | 4 | EXE71111.1 | 1812 | 8383 | 688 | EXE71113.1 | 1813 | 8384 | 200 | EXE71114.1 | 1814 | 8385 | 466 |
| 440 | I | 4 | KCX96450.1 | 1816 | 8387 | 686 | KCX96449.1 | 1817 | 8388 | 243 | KCX96448.1 | 1818 | 8389 | 466 |
| 441 | I | 4 | KHO17164.1 | 1820 | 8391 | 686 | KHO17163.1 | 1821 | 8392 | 243 | KHO17162.1 | 1822 | 8393 | 456 |
| 442 | I | 4 | KLT90624.1 | 1824 | 8395 | 686 | KLT90642.1 | 1825 | 8396 | 243 | KLT90662.1 | 1826 | 8397 | 456 |
| 443 | I | 4 | KRI43455.1 | 1828 | 8399 | 688 | KRI43456.1 | 1829 | 8400 | 243 | KRI43503.1 | 1830 | 8401 | 457 |
| 444 | I | 4 | KRJ35152.1 | 1832 | 8403 | 686 | KRJ35151.1 | 1833 | 8404 | 243 | KRJ35150.1 | 1834 | 8405 | 466 |
| 445 | I | 4 | AMN00863.1 | 1836 | 8407 | 686 | AMN00864.1 | 1837 | 8408 | 243 | AMN00865.1 | 1838 | 8409 | 466 |
| 446 | I | 4 | KDB58749.1 | 1840 | 8411 | 662 | KDB58628.1 | 1841 | 8412 | 234 | KDB58574.1 | 1842 | 8413 | 570 |
| 447 | I | 4 | KDD86366.1 | 1844 | 8415 | 669 | KDD86358.1 | 1845 | 8416 | 234 | KDD86345.1 | 1846 | 8417 | 570 |
| 448 | I | 4 | ACV76373.1 | 1848 | 8419 | 675 | ACV76374.1 | 1849 | 8420 | 273 | ACV76375.1 | 1850 | 8421 | 433 |
| 449 | I | 4 | AEI38596.1 | 1852 | 8423 | 675 | AEI38595.1 | 1853 | 8424 | 273 | AEI38594.1 | 1854 | 8425 | 433 |
| 450 | I | 4 | KTJ39354.1 | 1856 | 8427 | 729 | KTJ39355.1 | 1857 | 8428 | 246 | KTJ39356.1 | 1858 | 8429 | 559 |
| 451 | I | 4 | KUQ09076.1 | 1860 | 8431 | 851 | KUQ09077.1 | 1861 | 8432 | 245 | KUQ09078.1 | 1862 | 8433 | 597 |
| 452 | I | 4 | KYN64186.1 | 1864 | 8435 | 678 | KYN64185.1 | 1865 | 8436 | 287 | KYN64184.1 | 1866 | 8437 | 441 |
| 453 | I | 4 | AFP68831.1 | 1868 | 8439 | 729 | AFP68830.1 | 1869 | 8440 | 245 | AFP68829.1 | 1870 | 8441 | 70 |
| 454 | I | 4 | ESS59441.1 | 1872 | 8443 | 729 | ESS59449.1 | 1873 | 8444 | 208 | ESS59443.1 | 1874 | 8445 | 560 |
| 455 | I | 4 | EUL88641.1 | 1876 | 8447 | 711 | EUL88642.1 | 1877 | 8448 | 245 | EUL88643.1 | 1878 | 8449 | 560 |
| 456 | I | 4 | KDF77544.1 | 1880 | 8451 | 711 | KDF77543.1 | 1881 | 8452 | 245 | KDF77542.1 | 1882 | 8453 | 560 |
| 457 | I | 4 | AIX56040.1 | 1884 | 8455 | 729 | AIX56041.1 | 1885 | 8456 | 245 | AIX56042.1 | 1886 | 8457 | 504 |
| 458 | I | 4 | KJW77580.1 | 1888 | 8459 | 851 | KJW77579.1 | 1889 | 8460 | 245 | KJW77578.1 | 1890 | 8461 | 597 |
| 459 | I | 4 | KJX05616.1 | 1892 | 8463 | 851 | KJX05615.1 | 1893 | 8464 | 245 | KJX05614.1 | 1894 | 8465 | 597 |
| 460 | I | 3 | | | | | KPS87963.1 | 1896 | 8467 | 245 | KPS87962.1 | 1897 | 8468 | 560 |
| 461 | I | 3 | | | | | KRS24832.1 | 1899 | 8470 | 245 | KRS24833.1 | 1900 | 8471 | 560 |
| 462 | I | 4 | KUQ03195.1 | 1902 | 8473 | 729 | KUQ03194.1 | 1903 | 8474 | 245 | KUQ03193.1 | 1904 | 8475 | 560 |
| 463 | I | 4 | BAK13750.1 | 1906 | 8477 | 678 | BAK13749.1 | 1907 | 8478 | 287 | BAK13748.1 | 1908 | 8479 | 441 |
| 464 | I | 4 | CCF11168.1 | 1910 | 8481 | 678 | CCF11169.1 | 1911 | 8482 | 287 | CCF11170.1 | 1912 | 8483 | 356 |
| 465 | I | 4 | AFR04653.1 | 1914 | 8485 | 729 | AFR04654.1 | 1915 | 8486 | 246 | AFR04655.1 | 1916 | 8487 | 560 |
| 466 | I | 4 | KHS69362.1 | 1918 | 8489 | 729 | KHS69298.1 | 1919 | 8490 | 246 | KHS69299.1 | 1920 | 8491 | 560 |
| 467 | I | 4 | KHT02545.1 | 1922 | 8493 | 729 | KHT02546.1 | 1923 | 8494 | 246 | KHT02547.1 | 1924 | 8495 | 560 |
| 468 | I | 4 | KHT03993.1 | 1926 | 8497 | 729 | KHT03964.1 | 1927 | 8498 | 246 | KHT03965.1 | 1928 | 8499 | 560 |
| 469 | I | 4 | ABV17222.1 | 1930 | 8501 | 739 | ABV17786.1 | 1931 | 8502 | 246 | ABV16709.1 | 1932 | 8503 | 567 |
| 470 | I | 4 | EIP59422.1 | 1934 | 8505 | 729 | EIP59419.1 | 1935 | 8506 | 246 | EIP59433.1 | 1936 | 8507 | 483 |
| 471 | I | 4 | EIN79902.1 | 1938 | 8509 | 729 | EIN79901.1 | 1939 | 8510 | 246 | EIN79900.1 | 1940 | 8511 | 504 |
| 472 | I | 4 | EKK37352.1 | 1942 | 8513 | 729 | EKK37353.1 | 1943 | 8514 | 246 | EKK37354.1 | 1944 | 8515 | 504 |
| 473 | I | 4 | EKW33219.1 | 1946 | 8517 | 729 | EKW33218.1 | 1947 | 8518 | 246 | EKW33217.1 | 1948 | 8519 | 402 |
| 474 | I | 4 | ELW24564.1 | 1950 | 8521 | 729 | ELW24563.1 | 1951 | 8522 | 246 | ELW24562.1 | 1952 | 8523 | 483 |
| 475 | I | 4 | ELV55444.1 | 1954 | 8525 | 704 | ELV55443.1 | 1955 | 8526 | 246 | ELV55442.1 | 1956 | 8527 | 391 |
| 476 | I | 4 | ELV58670.1 | 1958 | 8529 | 729 | ELV58672.1 | 1959 | 8530 | 246 | ELV58675.1 | 1960 | 8531 | 483 |
| 477 | I | 4 | ELV27737.1 | 1962 | 8533 | 729 | ELV27736.1 | 1963 | 8534 | 246 | ELV27735.1 | 1964 | 8535 | 504 |
| 478 | I | 4 | ELE34850.1 | 1966 | 8537 | 729 | ELE34849.1 | 1967 | 8538 | 246 | ELE34848.1 | 1968 | 8539 | 560 |
| 479 | I | 4 | ELF60007.1 | 1970 | 8541 | 729 | ELF60008.1 | 1971 | 8542 | 246 | ELF60009.1 | 1972 | 8543 | 560 |
| 480 | I | 4 | ENC42752.1 | 1974 | 8545 | 729 | ENC42751.1 | 1975 | 8546 | 246 | ENC42750.1 | 1976 | 8547 | 504 |
| 481 | I | 4 | ENC49345.1 | 1978 | 8549 | 729 | ENC49344.1 | 1979 | 8550 | 246 | ENC49343.1 | 1980 | 8551 | 504 |
| 482 | I | 4 | ENC65372.1 | 1982 | 8553 | 665 | ENC65371.1 | 1983 | 8554 | 246 | ENC65370.1 | 1984 | 8555 | 504 |
| 483 | I | 4 | ENC60227.1 | 1986 | 8557 | 729 | ENC60226.1 | 1987 | 8558 | 246 | ENC60225.1 | 1988 | 8559 | 504 |
| 484 | I | 4 | ENC64694.1 | 1990 | 8561 | 729 | ENC64693.1 | 1991 | 8562 | 246 | ENC64692.1 | 1992 | 8563 | 504 |
| 485 | I | 4 | ENC77391.1 | 1994 | 8565 | 729 | ENC77390.1 | 1995 | 8566 | 246 | ENC77389.1 | 1996 | 8567 | 504 |
| 486 | I | 4 | ENC83691.1 | 1998 | 8569 | 729 | ENC83703.1 | 1999 | 8570 | 246 | ENC83675.1 | 2000 | 8571 | 504 |
| 487 | I | 4 | ENC86715.1 | 2002 | 8573 | 729 | ENC86714.1 | 2003 | 8574 | 246 | ENC86713.1 | 2004 | 8575 | 504 |
| 488 | I | 4 | ENE16651.1 | 2006 | 8577 | 729 | ENE16656.1 | 2007 | 8578 | 246 | ENE16652.1 | 2008 | 8579 | 560 |
| 489 | I | 4 | ENC77075.1 | 2010 | 8581 | 729 | ENC77074.1 | 2011 | 8582 | 246 | ENC77073.1 | 2012 | 8583 | 504 |
| 490 | I | 4 | EOQ56570.1 | 2014 | 8585 | 851 | EOQ56569.1 | 2015 | 8586 | 244 | EOQ56568.1 | 2016 | 8587 | 595 |
| 491 | I | 4 | ERB06047.1 | 2018 | 8589 | 729 | ERB06046.1 | 2019 | 8590 | 246 | ERB06045.1 | 2020 | 8591 | 560 |
| 492 | I | 4 | EQV94532.1 | 2022 | 8593 | 851 | EQV94533.1 | 2023 | 8594 | 244 | EQV94534.1 | 2024 | 8595 | 595 |
| 493 | I | 4 | EQV96721.1 | 2026 | 8597 | 729 | EQV96722.1 | 2027 | 8598 | 246 | EQV96723.1 | 2028 | 8599 | 560 |
| 494 | I | 4 | ERB23472.1 | 2030 | 8601 | 729 | ERB23473.1 | 2031 | 8602 | 246 | ERB23474.1 | 2032 | 8603 | 560 |
| 495 | I | 4 | ERA25533.1 | 2034 | 8605 | 729 | ERA25534.1 | 2035 | 8606 | 246 | ERA25535.1 | 2036 | 8607 | 560 |
| 496 | I | 4 | ERB97875.1 | 2038 | 8609 | 729 | ERB97874.1 | 2039 | 8610 | 246 | ERB97873.1 | 2040 | 8611 | 483 |
| 497 | I | 4 | ERB87466.1 | 2042 | 8613 | 729 | ERB87465.1 | 2043 | 8614 | 246 | ERB87464.1 | 2044 | 8615 | 483 |
| 498 | I | 4 | ERB73282.1 | 2046 | 8617 | 729 | ERB73281.1 | 2047 | 8618 | 246 | ERB73280.1 | 2048 | 8619 | 483 |
| 499 | I | 4 | ERB84327.1 | 2050 | 8621 | 729 | ERB84433.1 | 2051 | 8622 | 246 | ERB84435.1 | 2052 | 8623 | 483 |
| 500 | I | 4 | ERD03133.1 | 2054 | 8625 | 729 | ERD03126.1 | 2055 | 8626 | 246 | ERD03124.1 | 2056 | 8627 | 504 |
| 501 | I | 3 | | | | | ERD02933.1 | 2058 | 8629 | 175 | ERD02932.1 | 2059 | 8630 | 307 |
| 502 | I | 4 | ERD21118.1 | 2061 | 8632 | 729 | ERD21117.1 | 2062 | 8633 | 246 | ERD21116.1 | 2063 | 8634 | 483 |
| 503 | I | 4 | ERD19184.1 | 2065 | 8636 | 729 | ERD19183.1 | 2066 | 8637 | 246 | ERD19182.1 | 2067 | 8638 | 483 |
| 504 | I | 4 | ERD72949.1 | 2069 | 8640 | 729 | ERD72951.1 | 2070 | 8641 | 246 | ERD72947.1 | 2071 | 8642 | 483 |
| 505 | I | 4 | ERD66562.1 | 2073 | 8644 | 729 | ERD66561.1 | 2074 | 8645 | 246 | ERD66560.1 | 2075 | 8646 | 504 |
| 506 | I | 4 | ERD75978.1 | 2077 | 8648 | 729 | ERD75977.1 | 2078 | 8649 | 246 | ERD75976.1 | 2079 | 8650 | 483 |
| 507 | I | 4 | ERD83534.1 | 2081 | 8652 | 729 | ERD83533.1 | 2082 | 8653 | 246 | ERD83532.1 | 2083 | 8654 | 483 |
| 508 | I | 4 | ERD92971.1 | 2085 | 8656 | 729 | ERD92973.1 | 2086 | 8657 | 246 | ERD92974.1 | 2087 | 8658 | 483 |
| 509 | I | 4 | ERD99050.1 | 2089 | 8660 | 729 | ERD99051.1 | 2090 | 8661 | 246 | ERD99052.1 | 2091 | 8662 | 483 |
| 510 | I | 4 | ERD98127.1 | 2093 | 8664 | 729 | ERD98126.1 | 2094 | 8665 | 246 | ERD98125.1 | 2095 | 8666 | 504 |
| 511 | I | 4 | AHM42039.1 | 2097 | 8668 | 729 | AHM42040.1 | 2098 | 8669 | 246 | AHM42041.1 | 2099 | 8670 | 560 |
| 512 | I | 4 | AHM46674.1 | 2101 | 8672 | 729 | AHM46675.1 | 2102 | 8673 | 246 | AHM46676.1 | 2103 | 8674 | 560 |
| 513 | I | 4 | AHM51229.1 | 2105 | 8676 | 729 | AHM51230.1 | 2106 | 8677 | 246 | AHM51231.1 | 2107 | 8678 | 560 |
| 514 | I | 4 | EYD87612.1 | 2109 | 8680 | 698 | EYD87621.1 | 2110 | 8681 | 245 | EYD87618.1 | 2111 | 8682 | 595 |
| 515 | I | 4 | EYW22456.1 | 2113 | 8684 | 729 | EYW22455.1 | 2114 | 8685 | 246 | EYW22454.1 | 2115 | 8686 | 560 |
| 516 | I | 4 | EYW31324.1 | 2117 | 8688 | 729 | EYW31323.1 | 2118 | 8689 | 246 | EYW31322.1 | 2119 | 8690 | 560 |
| 517 | I | 4 | EYV86555.1 | 2121 | 8692 | 729 | EYV86554.1 | 2122 | 8693 | 246 | EYV86553.1 | 2123 | 8694 | 560 |
| 518 | I | 4 | EYW71956.1 | 2125 | 8696 | 729 | EYW71957.1 | 2126 | 8697 | 246 | EYW71958.1 | 2127 | 8698 | 560 |
| 519 | I | 4 | EYW62757.1 | 2129 | 8700 | 729 | EYW62758.1 | 2130 | 8701 | 246 | EYW62759.1 | 2131 | 8702 | 560 |
| 520 | I | 4 | EYW60244.1 | 2133 | 8704 | 729 | EYW60243.1 | 2134 | 8705 | 246 | EYW60242.1 | 2135 | 8706 | 560 |
| 521 | I | 4 | EYW56544.1 | 2137 | 8708 | 729 | EYW56543.1 | 2138 | 8709 | 246 | EYW56542.1 | 2139 | 8710 | 560 |
| 522 | I | 4 | EYW47186.1 | 2141 | 8712 | 729 | EYW47185.1 | 2142 | 8713 | 246 | EYW47184.1 | 2143 | 8714 | 560 |
| 523 | I | 4 | EYW47506.1 | 2145 | 8716 | 729 | EYW47505.1 | 2146 | 8717 | 246 | EYW47504.1 | 2147 | 8718 | 560 |
| 524 | I | 4 | EYW43756.1 | 2149 | 8720 | 729 | EYW43757.1 | 2150 | 8721 | 246 | EYW43758.1 | 2151 | 8722 | 560 |
| 525 | I | 4 | EYW40414.1 | 2153 | 8724 | 729 | EYW40415.1 | 2154 | 8725 | 246 | EYW40416.1 | 2155 | 8726 | 560 |
| 526 | I | 4 | EYW30112.1 | 2157 | 8728 | 729 | EYW30111.1 | 2158 | 8729 | 246 | EYW30110.1 | 2159 | 8730 | 560 |
| 527 | I | 4 | EYY79851.1 | 2161 | 8732 | 729 | EYY79852.1 | 2162 | 8733 | 246 | EYY79853.1 | 2163 | 8734 | 560 |
| 528 | I | 4 | EZB10673.1 | 2165 | 8736 | 729 | EZB10672.1 | 2166 | 8737 | 246 | EZB10671.1 | 2167 | 8738 | 560 |
| 529 | I | 4 | EZE32940.1 | 2169 | 8740 | 729 | EZE32941.1 | 2170 | 8741 | 246 | EZE32942.1 | 2171 | 8742 | 560 |
| 530 | I | 4 | KDZ82638.1 | 2173 | 8744 | 729 | KDZ82652.1 | 2174 | 8745 | 246 | KDZ82648.1 | 2175 | 8746 | 560 |
| 531 | I | 4 | KHG75769.1 | 2177 | 8748 | 729 | KHG75768.1 | 2178 | 8749 | 246 | KHG75767.1 | 2179 | 8750 | 560 |
| 532 | I | 4 | KHG79503.1 | 2181 | 8752 | 729 | KHG79502.1 | 2182 | 8753 | 246 | KHG79501.1 | 2183 | 8754 | 560 |
| 533 | I | 4 | KOZ37432.1 | 2185 | 8756 | 729 | KOZ37431.1 | 2186 | 8757 | 246 | KOZ37430.1 | 2187 | 8758 | 560 |
| 534 | I | 4 | KOZ53466.1 | 2189 | 8760 | 729 | KOZ53465.1 | 2190 | 8761 | 246 | KOZ53464.1 | 2191 | 8762 | 560 |
| 535 | I | 4 | KOZ92709.1 | 2193 | 8764 | 729 | KOZ92708.1 | 2194 | 8765 | 246 | KOZ92707.1 | 2195 | 8766 | 560 |
| 536 | I | 4 | CTV73490.1 | 2197 | 8768 | 729 | CTV73519.1 | 2198 | 8769 | 246 | CTV73555.1 | 2199 | 8770 | 560 |
| 537 | I | 4 | CTY75965.1 | 2201 | 8772 | 729 | CTY75938.1 | 2202 | 8773 | 246 | CTY75914.1 | 2203 | 8774 | 560 |
| 538 | I | 4 | KPH47568.1 | 2205 | 8776 | 729 | KPH47567.1 | 2206 | 8777 | 246 | KPH47566.1 | 2207 | 8778 | 560 |
| 539 | I | 4 | KPH29388.1 | 2209 | 8780 | 729 | KPH29389.1 | 2210 | 8781 | 246 | KPH29390.1 | 2211 | 8782 | 560 |
| 540 | I | 4 | KPO40054.1 | 2213 | 8784 | 729 | KPO40055.1 | 2214 | 8785 | 246 | KPO40056.1 | 2215 | 8786 | 560 |
| 541 | I | 4 | KPP40484.1 | 2217 | 8788 | 729 | KPP40485.1 | 2218 | 8789 | 246 | KPP40486.1 | 2219 | 8790 | 560 |
| 542 | I | 4 | KQI75364.1 | 2221 | 8792 | 729 | KQI75363.1 | 2222 | 8793 | 246 | KQI75362.1 | 2223 | 8794 | 560 |
| 543 | I | 4 | KST33199.1 | 2225 | 8796 | 729 | KST33200.1 | 2226 | 8797 | 246 | KST33201.1 | 2227 | 8798 | 560 |
| 544 | I | 4 | KUV50206.1 | 2229 | 8800 | 729 | KUV50205.1 | 2230 | 8801 | 246 | KUV50204.1 | 2231 | 8802 | 560 |
| 545 | I | 4 | KUT58773.1 | 2233 | 8804 | 729 | KUT58774.1 | 2234 | 8805 | 246 | KUT58775.1 | 2235 | 8806 | 560 |
| 546 | I | 4 | KXL70397.1 | 2237 | 8808 | 729 | KXL70398.1 | 2238 | 8809 | 246 | KXL70399.1 | 2239 | 8810 | 560 |
| 547 | I | 4 | KXM39825.1 | 2241 | 8812 | 729 | KXM39826.1 | 2242 | 8813 | 246 | KXM39827.1 | 2243 | 8814 | 560 |
| 548 | I | 4 | KXM53390.1 | 2245 | 8816 | 729 | KXM53391.1 | 2246 | 8817 | 246 | KXM53392.1 | 2247 | 8818 | 560 |
| 549 | I | 4 | KXN00948.1 | 2249 | 8820 | 729 | KXN00949.1 | 2250 | 8821 | 246 | KXN00950.1 | 2251 | 8822 | 560 |
| 550 | I | 4 | KXQ06776.1 | 2253 | 8824 | 729 | KXQ06777.1 | 2254 | 8825 | 246 | KXQ06778.1 | 2255 | 8826 | 560 |
| 551 | I | 4 | KXQ54353.1 | 2257 | 8828 | 729 | KXQ54354.1 | 2258 | 8829 | 246 | KXQ54355.1 | 2259 | 8830 | 560 |
| 552 | I | 4 | KXR15706.1 | 2261 | 8832 | 729 | KXR15705.1 | 2262 | 8833 | 246 | KXR15704.1 | 2263 | 8834 | 560 |
| 553 | I | 4 | KXQ76400.1 | 2265 | 8836 | 729 | KXQ76401.1 | 2266 | 8837 | 246 | KXQ76402.1 | 2267 | 8838 | 560 |
| 554 | I | 4 | KXQ99883.1 | 2269 | 8840 | 729 | KXQ99884.1 | 2270 | 8841 | 246 | KXQ99885.1 | 2271 | 8842 | 560 |
| 555 | I | 4 | KXR88728.1 | 2273 | 8844 | 729 | KXR88729.1 | 2274 | 8845 | 246 | KXR88730.1 | 2275 | 8846 | 560 |
| 556 | I | 4 | KXR78157.1 | 2277 | 8848 | 729 | KXR78156.1 | 2278 | 8849 | 246 | KXR78155.1 | 2279 | 8850 | 560 |
| 557 | I | 4 | EGC96566.1 | 2281 | 8852 | 716 | EGC96565.1 | 2282 | 8853 | 246 | EGC96564.1 | 2283 | 8854 | 560 |
| 558 | I | 4 | GAB54113.1 | 2285 | 8856 | 729 | GAB54114.1 | 2286 | 8857 | 246 | GAB54115.1 | 2287 | 8858 | 560 |
| 559 | I | 4 | EHM44110.1 | 2289 | 8860 | 729 | EHM44109.1 | 2290 | 8861 | 246 | EHM44108.1 | 2291 | 8862 | 560 |
| 560 | I | 4 | EKB79928.1 | 2293 | 8864 | 729 | EKB79929.1 | 2294 | 8865 | 247 | EKB79930.1 | 2295 | 8866 | 560 |
| 561 | I | 4 | EKB69927.1 | 2297 | 8868 | 729 | EKB69926.1 | 2298 | 8869 | 246 | EKB69925.1 | 2299 | 8870 | 560 |
| 562 | I | 4 | EMH92817.1 | 2301 | 8872 | 711 | EMH92791.1 | 2302 | 8873 | 246 | EMH92827.1 | 2303 | 8874 | 483 |
| 563 | I | 4 | EMH90435.1 | 2305 | 8876 | 729 | EMH90438.1 | 2306 | 8877 | 246 | EMH90437.1 | 2307 | 8878 | 560 |
| 564 | I | 4 | EPA89068.1 | 2309 | 8880 | 701 | EPA89053.1 | 2310 | 8881 | 247 | EPA89066.1 | 2311 | 8882 | 560 |
| 565 | I | 4 | EPO98337.1 | 2313 | 8884 | 701 | EPO98352.1 | 2314 | 8885 | 246 | EPO98313.1 | 2315 | 8886 | 560 |
| 566 | I | 4 | ERN60891.1 | 2317 | 8888 | 711 | ERN60892.1 | 2318 | 8889 | 247 | ERN60893.1 | 2319 | 8890 | 560 |
| 567 | I | 4 | ESM66670.1 | 2321 | 8892 | 729 | ESM66669.1 | 2322 | 8893 | 247 | ESM66668.1 | 2323 | 8894 | 560 |
| 568 | I | 3 | CDL16831.1 | 2325 | 8896 | 497 | CDL16832.1 | 2326 | 8897 | 247 | CDL16833.1 | 2327 | 8898 | 560 |
| 569 | I | 4 | EWE84467.1 | 2328 | 8899 | 729 | EWE84468.1 | 2329 | 8900 | 246 | EWE84469.1 | 2330 | 8901 | 560 |
| 570 | I | 4 | EWE89858.1 | 2332 | 8903 | 729 | EWE89859.1 | 2333 | 8904 | 247 | EWE89860.1 | 2334 | 8905 | 560 |
| 571 | I | 4 | EZQ78636.1 | 2336 | 8907 | 729 | EZQ78637.1 | 2337 | 8908 | 247 | EZQ78638.1 | 2338 | 8909 | 560 |
| 572 | I | 4 | EZR23958.1 | 2340 | 8911 | 729 | EZR23959.1 | 2341 | 8912 | 247 | EZR23960.1 | 2342 | 8913 | 560 |
| 573 | I | 4 | KDK71110.1 | 2344 | 8915 | 729 | KDK71111.1 | 2345 | 8916 | 247 | KDK71112.1 | 2346 | 8917 | 560 |
| 574 | I | 4 | KDL50055.1 | 2348 | 8919 | 729 | KDL50056.1 | 2349 | 8920 | 247 | KDL50057.1 | 2350 | 8921 | 560 |
| 575 | I | 4 | KEF24231.1 | 2352 | 8923 | 729 | KEF24232.1 | 2353 | 8924 | 247 | KEF24233.1 | 2354 | 8925 | 560 |
| 576 | I | 4 | AIG86121.1 | 2356 | 8927 | 711 | AIG86120.1 | 2357 | 8928 | 247 | AIG86119.1 | 2358 | 8929 | 521 |
| 577 | I | 4 | AIT02041.1 | 2360 | 8931 | 729 | AIT02042.1 | 2361 | 8932 | 247 | AIT02043.1 | 2362 | 8933 | 560 |
| 578 | I | 4 | KGK55644.1 | 2364 | 8935 | 729 | KGK55905.1 | 2365 | 8936 | 246 | KGK55643.1 | 2366 | 8937 | 560 |
| 579 | I | 4 | KHF56285.1 | 2368 | 8939 | 729 | KHF56286.1 | 2369 | 8940 | 247 | KHF56287.1 | 2370 | 8941 | 560 |
| 580 | I | 4 | CEO84459.1 | 2372 | 8943 | 729 | CEO84460.1 | 2373 | 8944 | 247 | CEO84461.1 | 2374 | 8945 | 560 |
| 581 | I | 4 | KLZ85284.1 | 2376 | 8947 | 729 | KLZ85285.1 | 2377 | 8948 | 247 | KLZ85286.1 | 2378 | 8949 | 560 |
| 582 | I | 4 | KME72626.1 | 2380 | 8951 | 729 | KME72627.1 | 2381 | 8952 | 246 | KME72628.1 | 2382 | 8953 | 560 |
| 583 | I | 4 | KMG80267.1 | 2384 | 8955 | 729 | KMG80268.1 | 2385 | 8956 | 247 | KMG80269.1 | 2386 | 8957 | 560 |
| 584 | I | 4 | KMH33138.1 | 2388 | 8959 | 729 | KMH33139.1 | 2389 | 8960 | 247 | KMH33140.1 | 2390 | 8961 | 560 |
| 585 | I | 4 | KMH45006.1 | 2392 | 8963 | 729 | KMH45007.1 | 2393 | 8964 | 247 | KMH45008.1 | 2394 | 8965 | 560 |
| 586 | I | 4 | KMH61874.1 | 2396 | 8967 | 729 | KMH61875.1 | 2397 | 8968 | 247 | KMH61876.1 | 2398 | 8969 | 560 |
| 587 | I | 4 | KMH74805.1 | 2400 | 8971 | 729 | KMH74806.1 | 2401 | 8972 | 247 | KMH74807.1 | 2402 | 8973 | 560 |
| 588 | I | 4 | KMH92291.1 | 2404 | 8975 | 729 | KMH92292.1 | 2405 | 8976 | 247 | KMH92293.1 | 2406 | 8977 | 560 |
| 589 | I | 4 | KMH82199.1 | 2408 | 8979 | 729 | KMH82198.1 | 2409 | 8980 | 246 | KMH82197.1 | 2410 | 8981 | 560 |
| 590 | I | 4 | KMI35044.1 | 2412 | 8983 | 729 | KMI35043.1 | 2413 | 8984 | 246 | KMI35042.1 | 2414 | 8985 | 560 |
| 591 | I | 4 | KMJ00751.1 | 2416 | 8987 | 729 | KMJ00752.1 | 2417 | 8988 | 247 | KMJ00753.1 | 2418 | 8989 | 560 |
| 592 | I | 4 | KMX47009.1 | 2420 | 8991 | 729 | KMX47010.1 | 2421 | 8992 | 247 | KMX47011.1 | 2422 | 8993 | 560 |
| 593 | I | 4 | ALH85688.1 | 2424 | 8995 | 729 | ALH88375.1 | 2425 | 8996 | 246 | ALH85687.1 | 2426 | 8997 | 560 |
| 594 | I | 4 | KPR26030.1 | 2428 | 8999 | 729 | KPR26088.1 | 2429 | 9000 | 246 | KPR26029.1 | 2430 | 9001 | 560 |
| 595 | I | 4 | KSW78890.1 | 2432 | 9003 | 851 | KSW78889.1 | 2433 | 9004 | 245 | KSW78888.1 | 2434 | 9005 | 627 |
| 596 | I | 4 | ALP77147.1 | 2436 | 9007 | 729 | ALP81045.1 | 2437 | 9008 | 246 | ALP77148.1 | 2438 | 9009 | 560 |
| 597 | I | 4 | KSZ25786.1 | 2440 | 9011 | 729 | KSZ25847.1 | 2441 | 9012 | 246 | KSZ25785.1 | 2442 | 9013 | 560 |
| 598 | I | 4 | AMA16471.1 | 2444 | 9015 | 729 | AMA19153.1 | 2445 | 9016 | 246 | AMA16470.1 | 2446 | 9017 | 560 |
| 599 | I | 4 | KXO21002.1 | 2448 | 9019 | 729 | KXO21035.1 | 2449 | 9020 | 246 | KXO21003.1 | 2450 | 9021 | 560 |
| 600 | I | 4 | KYL54018.1 | 2452 | 9023 | 729 | KYL54030.1 | 2453 | 9024 | 246 | KYL54017.1 | 2454 | 9025 | 560 |
| 601 | I | 4 | KYM08970.1 | 2456 | 9027 | 729 | KYM08978.1 | 2457 | 9028 | 246 | KYM08971.1 | 2458 | 9029 | 560 |
| 602 | I | 4 | KYL42995.1 | 2460 | 9031 | 729 | KYL43003.1 | 2461 | 9032 | 246 | KYL42996.1 | 2462 | 9033 | 560 |
| 603 | I | 4 | KYL42676.1 | 2464 | 9035 | 729 | KYL42684.1 | 2465 | 9036 | 246 | KYL42677.1 | 2466 | 9037 | 560 |
| 604 | I | 4 | KYL71102.1 | 2468 | 9039 | 729 | KYL71114.1 | 2469 | 9040 | 246 | KYL71101.1 | 2470 | 9041 | 560 |
| 605 | I | 4 | KYL53998.1 | 2472 | 9043 | 729 | KYL54010.1 | 2473 | 9044 | 246 | KYL53997.1 | 2474 | 9045 | 560 |
| 606 | I | 4 | KYM33518.1 | 2476 | 9047 | 729 | KYM33585.1 | 2477 | 9048 | 246 | KYM33517.1 | 2478 | 9049 | 560 |
| 607 | I | 4 | KYL73344.1 | 2480 | 9051 | 729 | KYL73352.1 | 2481 | 9052 | 246 | KYL73345.1 | 2482 | 9053 | 560 |
| 608 | I | 4 | KYL53874.1 | 2484 | 9055 | 729 | KYL53882.1 | 2485 | 9056 | 246 | KYL53875.1 | 2486 | 9057 | 560 |
| 609 | I | 4 | KYM21786.1 | 2488 | 9059 | 729 | KYM21794.1 | 2489 | 9060 | 246 | KYM21787.1 | 2490 | 9061 | 560 |
| 610 | I | 4 | KYM07148.1 | 2492 | 9063 | 729 | KYM07160.1 | 2493 | 9064 | 246 | KYM07147.1 | 2494 | 9065 | 560 |
| 611 | I | 4 | KYM09278.1 | 2496 | 9067 | 729 | KYM09290.1 | 2497 | 9068 | 246 | KYM09277.1 | 2498 | 9069 | 560 |
| 612 | I | 4 | KYL81720.1 | 2500 | 9071 | 729 | KYL81728.1 | 2501 | 9072 | 246 | KYL81721.1 | 2502 | 9073 | 560 |
| 613 | I | 4 | KYL90348.1 | 2504 | 9075 | 729 | KYL90356.1 | 2505 | 9076 | 246 | KYL90349.1 | 2506 | 9077 | 560 |
| 614 | I | 4 | KYM24956.1 | 2508 | 9079 | 729 | KYM24969.1 | 2509 | 9080 | 246 | KYM24955.1 | 2510 | 9081 | 560 |
| 615 | I | 4 | CQJ58381.1 | 2512 | 9083 | 678 | CQJ58386.1 | 2513 | 9084 | 287 | CQJ58391.1 | 2514 | 9085 | 442 |
| 616 | I | 4 | CFV22988.1 | 2516 | 9087 | 678 | CFV22985.1 | 2517 | 9088 | 287 | CFV22982.1 | 2518 | 9089 | 442 |
| 617 | I | 4 | CNI05577.1 | 2520 | 9091 | 678 | CNI05611.1 | 2521 | 9092 | 287 | CNI05642.1 | 2522 | 9093 | 442 |
| 618 | I | 4 | AHV33806.1 | 2524 | 9095 | 686 | AHV33807.1 | 2525 | 9096 | 255 | AHV33808.1 | 2526 | 9097 | 686 |
| 619 | I | 4 | KOG92701.1 | 2528 | 9099 | 686 | KOG92700.1 | 2529 | 9100 | 255 | KOG92699.1 | 2530 | 9101 | 686 |
| 620 | I | 4 | KRW58412.1 | 2532 | 9103 | 682 | KRW58411.1 | 2533 | 9104 | 236 | KRW58410.1 | 2534 | 9105 | 567 |
| 621 | I | 4 | AAF94913.1 | 2536 | 9107 | 706 | AAF94912.1 | 2537 | 9108 | 244 | AAF94911.1 | 2538 | 9109 | 483 |
| 622 | I | 4 | ABQ21745.1 | 2540 | 9111 | 706 | ABQ19942.1 | 2541 | 9112 | 244 | ABQ20511.1 | 2542 | 9113 | 483 |
| 623 | I | 4 | ACP06009.1 | 2544 | 9115 | 706 | ACP06008.1 | 2545 | 9116 | 244 | ACP06007.1 | 2546 | 9117 | 483 |
| 624 | I | 4 | ACP09874.1 | 2548 | 9119 | 706 | ACP09873.1 | 2549 | 9120 | 244 | ACP09872.1 | 2550 | 9121 | 483 |
| 625 | I | 3 | ACQ60772.1 | 2552 | 9123 | 706 | ACQ60773.1 | 2553 | 9124 | 244 | ACQ60774.1 | 2554 | 9125 | 483 |
| 626 | I | 4 | EFH78549.1 | 2555 | 9126 | 706 | EFH78550.1 | 2556 | 9127 | 244 | EFH78551.1 | 2557 | 9128 | 483 |
| 627 | I | 3 | | | | | EAZ72428.1 | 2559 | 9130 | 244 | EAZ72422.1 | 2560 | 9131 | 483 |
| 628 | I | 4 | EAZ78581.1 | 2562 | 9133 | 706 | EAZ78546.1 | 2563 | 9134 | 244 | EAZ78565.1 | 2564 | 9135 | 483 |
| 629 | I | 4 | AET26859.1 | 2566 | 9137 | 706 | AET26858.1 | 2567 | 9138 | 244 | AET26857.1 | 2568 | 9139 | 483 |
| 630 | I | 4 | KNH51113.1 | 2570 | 9141 | 706 | KNH51114.1 | 2571 | 9142 | 244 | KNH51115.1 | 2572 | 9143 | 483 |
| 631 | I | 4 | KNA61358.1 | 2574 | 9145 | 706 | KNA61359.1 | 2575 | 9146 | 244 | KNA61360.1 | 2576 | 9147 | 483 |
| 632 | I | 4 | EEO08431.1 | 2578 | 9149 | 706 | EEO08430.1 | 2579 | 9150 | 244 | EEO08429.1 | 2580 | 9151 | 483 |
| 633 | I | 4 | EEO16534.1 | 2582 | 9153 | 706 | EEO16533.1 | 2583 | 9154 | 244 | EEO16532.1 | 2584 | 9155 | 483 |
| 634 | I | 4 | EEO20559.1 | 2586 | 9157 | 706 | EEO20560.1 | 2587 | 9158 | 244 | EEO20561.1 | 2588 | 9159 | 483 |
| 635 | I | 4 | EET92687.1 | 2590 | 9161 | 706 | EET92688.1 | 2591 | 9162 | 244 | EET92689.1 | 2592 | 9163 | 483 |
| 636 | I | 4 | EEY40708.1 | 2594 | 9165 | 706 | EEY40707.1 | 2595 | 9166 | 244 | EEY40706.1 | 2596 | 9167 | 483 |
| 637 | I | 4 | EEY47283.1 | 2598 | 9169 | 687 | EEY47282.1 | 2599 | 9170 | 244 | EEY47281.1 | 2600 | 9171 | 483 |
| 638 | I | 4 | EGQ97277.1 | 2602 | 9173 | 687 | EGQ97276.1 | 2603 | 9174 | 244 | EGQ97275.1 | 2604 | 9175 | 483 |
| 639 | I | 4 | EGQ98251.1 | 2606 | 9177 | 706 | EGQ98250.1 | 2607 | 9178 | 244 | EGQ98249.1 | 2608 | 9179 | 483 |
| 640 | I | 4 | EGS48435.1 | 2610 | 9181 | 706 | EGS48434.1 | 2611 | 9182 | 244 | EGS48433.1 | 2612 | 9183 | 483 |
| 641 | I | 4 | EGS47646.1 | 2614 | 9185 | 706 | EGS47645.1 | 2615 | 9186 | 244 | EGS47644.1 | 2616 | 9187 | 483 |
| 642 | I | 4 | EGS47807.1 | 2618 | 9189 | 706 | EGS47806.1 | 2619 | 9190 | 244 | EGS47805.1 | 2620 | 9191 | 483 |
| 643 | I | 4 | EGS62508.1 | 2622 | 9193 | 687 | EGS62507.1 | 2623 | 9194 | 244 | EGS62506.1 | 2624 | 9195 | 483 |
| 644 | I | 4 | EGS70775.1 | 2626 | 9197 | 706 | EGS70774.1 | 2627 | 9198 | 244 | EGS70773.1 | 2628 | 9199 | 483 |
| 645 | I | 4 | EHH71591.1 | 2630 | 9201 | 706 | EHH71590.1 | 2631 | 9202 | 244 | EHH71589.1 | 2632 | 9203 | 483 |
| 646 | I | 4 | EHH83258.1 | 2634 | 9205 | 706 | EHH83257.1 | 2635 | 9206 | 244 | EHH83256.1 | 2636 | 9207 | 483 |
| 647 | I | 4 | EHH87250.1 | 2638 | 9209 | 706 | EHH87249.1 | 2639 | 9210 | 244 | EHH87248.1 | 2640 | 9211 | 483 |
| 648 | I | 4 | EHH97392.1 | 2642 | 9213 | 706 | EHH97391.1 | 2643 | 9214 | 244 | EHH97390.1 | 2644 | 9215 | 483 |
| 649 | I | 4 | EHI04662.1 | 2646 | 9217 | 706 | EHI05345.1 | 2647 | 9218 | 218 | EHI05392.1 | 2648 | 9219 | 483 |
| 650 | I | 4 | EHI04020.1 | 2650 | 9221 | 706 | EHI04019.1 | 2651 | 9222 | 244 | EHI04018.1 | 2652 | 9223 | 214 |
| 651 | I | 4 | EHH99557.1 | 2654 | 9225 | 706 | EHH99556.1 | 2655 | 9226 | 244 | EHH99555.1 | 2656 | 9227 | 472 |
| 652 | I | 4 | EHH93201.1 | 2658 | 9229 | 706 | EHH93200.1 | 2659 | 9230 | 244 | EHH93199.1 | 2660 | 9231 | 483 |
| 653 | I | 4 | EHH82145.1 | 2662 | 9233 | 706 | EHH82144.1 | 2663 | 9234 | 244 | EHH82143.1 | 2664 | 9235 | 483 |
| 654 | I | 4 | EHH72367.1 | 2666 | 9237 | 706 | EHH72366.1 | 2667 | 9238 | 244 | EHH72365.1 | 2668 | 9239 | 483 |
| 655 | I | 4 | EHH76760.1 | 2670 | 9241 | 706 | EHH76759.1 | 2671 | 9242 | 244 | EHH76758.1 | 2672 | 9243 | 483 |
| 656 | I | 4 | AFC58582.1 | 2674 | 9245 | 706 | AFC58581.1 | 2675 | 9246 | 244 | AFC58580.1 | 2676 | 9247 | 483 |
| 657 | I | 4 | EJH73152.1 | 2678 | 9249 | 706 | EJH73151.1 | 2679 | 9250 | 244 | EJH73150.1 | 2680 | 9251 | 483 |
| 658 | I | 4 | EJH69792.1 | 2682 | 9253 | 706 | EJH69791.1 | 2683 | 9254 | 244 | EJH69790.1 | 2684 | 9255 | 483 |
| 659 | I | 4 | EJH28006.1 | 2686 | 9257 | 706 | EJH28005.1 | 2687 | 9258 | 218 | EJH28004.1 | 2688 | 9259 | 483 |
| 660 | I | 4 | EJH27267.1 | 2690 | 9261 | 706 | EJH27423.1 | 2691 | 9262 | 244 | EJH27099.1 | 2692 | 9263 | 483 |
| 661 | I | 4 | EJH29363.1 | 2694 | 9265 | 706 | EJH29362.1 | 2695 | 9266 | 218 | EJH29361.1 | 2696 | 9267 | 451 |
| 662 | I | 4 | EJH38300.1 | 2698 | 9269 | 706 | EJH38299.1 | 2699 | 9270 | 244 | EJH38298.1 | 2700 | 9271 | 483 |
| 663 | I | 4 | EJH70293.1 | 2702 | 9273 | 706 | EJH70292.1 | 2703 | 9274 | 244 | EJH70291.1 | 2704 | 9275 | 483 |
| 664 | I | 4 | EJH44250.1 | 2706 | 9277 | 706 | EJH44249.1 | 2707 | 9278 | 218 | EJH44248.1 | 2708 | 9279 | 483 |
| 665 | I | 4 | EJH55113.1 | 2710 | 9281 | 706 | EJH55112.1 | 2711 | 9282 | 244 | EJH55111.1 | 2712 | 9283 | 483 |
| 666 | I | 4 | EJH55194.1 | 2714 | 9285 | 706 | EJH55227.1 | 2715 | 9286 | 218 | EJH55313.1 | 2716 | 9287 | 483 |
| 667 | I | 4 | EJH83358.1 | 2718 | 9289 | 706 | EJH83357.1 | 2719 | 9290 | 244 | EJH83356.1 | 2720 | 9291 | 483 |
| 668 | I | 4 | EJH85693.1 | 2722 | 9293 | 706 | EJH85692.1 | 2723 | 9294 | 244 | EJH85691.1 | 2724 | 9295 | 483 |
| 669 | I | 4 | EJH80829.1 | 2726 | 9297 | 706 | EJH80828.1 | 2727 | 9298 | 244 | EJH80827.1 | 2728 | 9299 | 483 |
| 670 | I | 4 | EJH40245.1 | 2730 | 9301 | 706 | EJH40244.1 | 2731 | 9302 | 244 | EJH40243.1 | 2732 | 9303 | 483 |
| 671 | I | 4 | EKG51169.1 | 2734 | 9305 | 706 | EKG51168.1 | 2735 | 9306 | 244 | EKG51167.1 | 2736 | 9307 | 483 |
| 672 | I | 4 | EKG45548.1 | 2738 | 9309 | 706 | EKG45547.1 | 2739 | 9310 | 244 | EKG45546.1 | 2740 | 9311 | 483 |
| 673 | I | 4 | EKG69364.1 | 2742 | 9313 | 706 | EKG69363.1 | 2743 | 9314 | 244 | EKG69362.1 | 2744 | 9315 | 483 |
| 674 | I | 4 | EKG78207.1 | 2746 | 9317 | 706 | EKG78206.1 | 2747 | 9318 | 244 | EKG78205.1 | 2748 | 9319 | 483 |
| 675 | I | 4 | EKG79527.1 | 2750 | 9321 | 687 | EKG79526.1 | 2751 | 9322 | 244 | EKG79525.1 | 2752 | 9323 | 483 |
| 676 | I | 4 | EKG89065.1 | 2754 | 9325 | 687 | EKG89064.1 | 2755 | 9326 | 218 | EKG89063.1 | 2756 | 9327 | 483 |
| 677 | I | 4 | EKK80943.1 | 2758 | 9329 | 706 | EKK80942.1 | 2759 | 9330 | 244 | EKK80941.1 | 2760 | 9331 | 483 |
| 678 | I | 4 | EKK97423.1 | 2762 | 9333 | 687 | EKK97422.1 | 2763 | 9334 | 244 | EKK97421.1 | 2764 | 9335 | 483 |
| 679 | I | 4 | EKL07579.1 | 2766 | 9337 | 318 | EKL07578.1 | 2767 | 9338 | 244 | EKL07615.1 | 2768 | 9339 | 265 |
| 680 | I | 4 | EKL26689.1 | 2770 | 9341 | 706 | EKL26688.1 | 2771 | 9342 | 244 | EKL26687.1 | 2772 | 9343 | 483 |
| 681 | I | 4 | EKL25950.1 | 2774 | 9345 | 706 | EKL25949.1 | 2775 | 9346 | 244 | EKL25948.1 | 2776 | 9347 | 483 |
| 682 | I | 4 | EKL86050.1 | 2778 | 9349 | 706 | EKL86049.1 | 2779 | 9350 | 244 | EKL86048.1 | 2780 | 9351 | 483 |
| 683 | I | 4 | EKM09914.1 | 2782 | 9353 | 706 | EKM09901.1 | 2783 | 9354 | 244 | EKM09801.1 | 2784 | 9355 | 483 |
| 684 | I | 4 | EKL86446.1 | 2786 | 9357 | 687 | EKL86445.1 | 2787 | 9358 | 244 | EKL86444.1 | 2788 | 9359 | 483 |
| 685 | I | 4 | EKM18416.1 | 2790 | 9361 | 687 | EKM18415.1 | 2791 | 9362 | 244 | EKM18414.1 | 2792 | 9363 | 483 |
| 686 | I | 4 | ELT24501.1 | 2794 | 9365 | 687 | ELT24500.1 | 2795 | 9366 | 218 | ELT24499.1 | 2796 | 9367 | 451 |
| 687 | I | 4 | ELT39817.1 | 2798 | 9369 | 706 | ELT39816.1 | 2799 | 9370 | 244 | ELT39815.1 | 2800 | 9371 | 483 |
| 688 | I | 4 | ELS74167.1 | 2802 | 9373 | 706 | ELS74166.1 | 2803 | 9374 | 244 | ELS74165.1 | 2804 | 9375 | 483 |
| 689 | I | 4 | ELS81598.1 | 2806 | 9377 | 687 | ELS81597.1 | 2807 | 9378 | 244 | ELS81596.1 | 2808 | 9379 | 483 |
| 690 | I | 4 | ELS88997.1 | 2810 | 9381 | 706 | ELS88996.1 | 2811 | 9382 | 244 | ELS88995.1 | 2812 | 9383 | 483 |
| 691 | I | 4 | ELS96628.1 | 2814 | 9385 | 706 | ELS96627.1 | 2815 | 9386 | 244 | ELS96626.1 | 2816 | 9387 | 483 |
| 692 | I | 4 | ELT03894.1 | 2818 | 9389 | 706 | ELT03893.1 | 2819 | 9390 | 244 | ELT03892.1 | 2820 | 9391 | 483 |
| 693 | I | 4 | ELT10277.1 | 2822 | 9393 | 687 | ELT10276.1 | 2823 | 9394 | 244 | ELT10275.1 | 2824 | 9395 | 483 |
| 694 | I | 4 | ELT32501.1 | 2826 | 9397 | 706 | ELT32500.1 | 2827 | 9398 | 244 | ELT32499.1 | 2828 | 9399 | 483 |
| 695 | I | 4 | EMB02074.1 | 2830 | 9401 | 706 | EMB02075.1 | 2831 | 9402 | 244 | EMB02076.1 | 2832 | 9403 | 483 |
| 696 | I | 4 | EMP84497.1 | 2834 | 9405 | 706 | EMP84506.1 | 2835 | 9406 | 244 | EMP84573.1 | 2836 | 9407 | 483 |
| 697 | I | 4 | EMP97717.1 | 2838 | 9409 | 687 | EMP97716.1 | 2839 | 9410 | 244 | EMP97715.1 | 2840 | 9411 | 483 |
| 698 | I | 4 | EMP95638.1 | 2842 | 9413 | 706 | EMP95637.1 | 2843 | 9414 | 244 | EMP95636.1 | 2844 | 9415 | 483 |
| 699 | I | 4 | EMQ03274.1 | 2846 | 9417 | 706 | EMQ03273.1 | 2847 | 9418 | 244 | EMQ03272.1 | 2848 | 9419 | 483 |
| 700 | I | 4 | EMQ04749.1 | 2850 | 9421 | 687 | EMQ04748.1 | 2851 | 9422 | 244 | EMQ04747.1 | 2852 | 9423 | 483 |
| 701 | I | 4 | EMQ11843.1 | 2854 | 9425 | 687 | EMQ11842.1 | 2855 | 9426 | 244 | EMQ11841.1 | 2856 | 9427 | 483 |
| 702 | I | 4 | EMQ11497.1 | 2858 | 9429 | 706 | EMQ11496.1 | 2859 | 9430 | 244 | EMQ11495.1 | 2860 | 9431 | 483 |
| 703 | I | 4 | EMQ16257.1 | 2862 | 9433 | 687 | EMQ16256.1 | 2863 | 9434 | 244 | EMQ16255.1 | 2864 | 9435 | 483 |
| 704 | I | 4 | EMQ22003.1 | 2866 | 9437 | 706 | EMQ22002.1 | 2867 | 9438 | 244 | EMQ22001.1 | 2868 | 9439 | 483 |
| 705 | I | 4 | EMQ27293.1 | 2870 | 9441 | 706 | EMQ27292.1 | 2871 | 9442 | 244 | EMQ27291.1 | 2872 | 9443 | 483 |
| 706 | I | 4 | EMQ26220.1 | 2874 | 9445 | 706 | EMQ26219.1 | 2875 | 9446 | 244 | EMQ26218.1 | 2876 | 9447 | 483 |
| 707 | I | 4 | EMQ32320.1 | 2878 | 9449 | 706 | EMQ32319.1 | 2879 | 9450 | 244 | EMQ32318.1 | 2880 | 9451 | 483 |
| 708 | I | 4 | EMQ37900.1 | 2882 | 9453 | 706 | EMQ37899.1 | 2883 | 9454 | 218 | EMQ37898.1 | 2884 | 9455 | 483 |
| 709 | I | 4 | EMQ51395.1 | 2886 | 9457 | 706 | EMQ51394.1 | 2887 | 9458 | 244 | EMQ51393.1 | 2888 | 9459 | 483 |
| 710 | I | 4 | EMQ53996.1 | 2890 | 9461 | 706 | EMQ53995.1 | 2891 | 9462 | 244 | EMQ53994.1 | 2892 | 9463 | 483 |
| 711 | I | 4 | EMQ39198.1 | 2894 | 9465 | 687 | EMQ39197.1 | 2895 | 9466 | 244 | EMQ39196.1 | 2896 | 9467 | 483 |
| 712 | I | 4 | EMQ68296.1 | 2898 | 9469 | 687 | EMQ68295.1 | 2899 | 9470 | 244 | EMQ68294.1 | 2900 | 9471 | 483 |
| 713 | I | 4 | EMQ62929.1 | 2902 | 9473 | 687 | EMQ62928.1 | 2903 | 9474 | 244 | EMQ62927.1 | 2904 | 9475 | 483 |
| 714 | I | 4 | EMQ43439.1 | 2906 | 9477 | 706 | EMQ43438.1 | 2907 | 9478 | 244 | EMQ43437.1 | 2908 | 9479 | 483 |
| 715 | I | 4 | EMQ47715.1 | 2910 | 9481 | 706 | EMQ47714.1 | 2911 | 9482 | 244 | EMQ47713.1 | 2912 | 9483 | 483 |
| 716 | I | 4 | EWM32711.1 | 2914 | 9485 | 706 | EWM32710.1 | 2915 | 9486 | 244 | EWM32709.1 | 2916 | 9487 | 483 |
| 717 | I | 4 | KEA49020.1 | 2918 | 9489 | 706 | KEA49019.1 | 2919 | 9490 | 244 | KEA49018.1 | 2920 | 9491 | 483 |
| 718 | I | 3 | KEH05440.1 | 2922 | 9493 | 706 | KEH05439.1 | 2923 | 9494 | 244 | KEH05438.1 | 2924 | 9495 | 483 |
| 719 | I | 4 | KEH80152.1 | 2925 | 9496 | 706 | KEH80153.1 | 2926 | 9497 | 244 | KEH80154.1 | 2927 | 9498 | 483 |
| 720 | I | 4 | KEH82623.1 | 2929 | 9500 | 706 | KEH82624.1 | 2930 | 9501 | 244 | KEH82625.1 | 2931 | 9502 | 483 |
| 721 | I | 3 | KFE04003.1 | 2933 | 9504 | 569 | KFE04002.1 | 2934 | 9505 | 244 | KFE04000.1 | 2935 | 9506 | 483 |
| 722 | I | 3 | KFE07491.1 | 2936 | 9507 | 569 | KFE07489.1 | 2937 | 9508 | 244 | KFE07492.1 | 2938 | 9509 | 483 |
| 723 | I | 4 | KFE03707.1 | 2939 | 9510 | 687 | KFE03742.1 | 2940 | 9511 | 244 | KFE03727.1 | 2941 | 9512 | 483 |
| 724 | I | 4 | KFE20316.1 | 2943 | 9514 | 687 | KFE20291.1 | 2944 | 9515 | 244 | KFE20317.1 | 2945 | 9516 | 483 |
| 725 | I | 4 | KFZ34215.1 | 2947 | 9518 | 706 | KFZ34214.1 | 2948 | 9519 | 244 | KFZ34213.1 | 2949 | 9520 | 483 |
| 726 | I | 4 | AIT29756.1 | 2951 | 9522 | 706 | AIT29755.1 | 2952 | 9523 | 244 | AIT29754.1 | 2953 | 9524 | 483 |
| 727 | I | 4 | KHE18844.1 | 2955 | 9526 | 706 | KHE18843.1 | 2956 | 9527 | 244 | KHE18842.1 | 2957 | 9528 | 483 |
| 728 | I | 4 | KHE21916.1 | 2959 | 9530 | 706 | KHE21917.1 | 2960 | 9531 | 244 | KHE21918.1 | 2961 | 9532 | 483 |
| 729 | I | 4 | BAP03192.1 | 2963 | 9534 | 706 | BAP03191.1 | 2964 | 9535 | 244 | BAP03190.1 | 2965 | 9536 | 483 |
| 730 | I | 4 | KJD16371.1 | 2967 | 9538 | 706 | KJD16372.1 | 2968 | 9539 | 244 | KJD16373.1 | 2969 | 9540 | 483 |
| 731 | I | 4 | KJJ52420.1 | 2971 | 9542 | 706 | KJJ52419.1 | 2972 | 9543 | 244 | KJJ52418.1 | 2973 | 9544 | 483 |
| 732 | I | 4 | KJJ54929.1 | 2975 | 9546 | 706 | KJJ54928.1 | 2976 | 9547 | 244 | KJJ54927.1 | 2977 | 9548 | 483 |
| 733 | I | 4 | KJR76013.1 | 2979 | 9550 | 706 | KJR76012.1 | 2980 | 9551 | 244 | KJR76011.1 | 2981 | 9552 | 483 |
| 734 | I | 4 | KJS92997.1 | 2983 | 9554 | 706 | KJS92996.1 | 2984 | 9555 | 244 | KJS92995.1 | 2985 | 9556 | 483 |
| 735 | I | 4 | AKA00225.1 | 2987 | 9558 | 706 | AKA00224.1 | 2988 | 9559 | 244 | AKA00223.1 | 2989 | 9560 | 483 |
| 736 | I | 4 | KJW14527.1 | 2991 | 9562 | 706 | KJW14526.1 | 2992 | 9563 | 244 | KJW14525.1 | 2993 | 9564 | 483 |
| 737 | I | 4 | KJX83381.1 | 2995 | 9566 | 706 | KJX83382.1 | 2996 | 9567 | 244 | KJX83383.1 | 2997 | 9568 | 483 |
| 738 | I | 4 | KKP13603.1 | 2999 | 9570 | 687 | KKP13602.1 | 3000 | 9571 | 218 | KKP13601.1 | 3001 | 9572 | 483 |
| 739 | I | 4 | KKP11543.1 | 3003 | 9574 | 706 | KKP11542.1 | 3004 | 9575 | 244 | KKP11541.1 | 3005 | 9576 | 472 |
| 740 | I | 4 | KKP10397.1 | 3007 | 9578 | 706 | KKP10398.1 | 3008 | 9579 | 218 | KKP10399.1 | 3009 | 9580 | 472 |
| 741 | I | 4 | AKO75286.1 | 3011 | 9582 | 706 | AKO75285.1 | 3012 | 9583 | 244 | AKO75284.1 | 3013 | 9584 | 483 |
| 742 | I | 3 | | | | | CSC37237.1 | 3015 | 9586 | 244 | CSC37265.1 | 3016 | 9587 | 492 |
| 743 | I | 4 | CRZ83881.1 | 3018 | 9589 | 687 | CRZ83843.1 | 3019 | 9590 | 244 | CRZ83797.1 | 3020 | 9591 | 166 |
| 744 | I | 4 | CSE02212.1 | 3022 | 9593 | 687 | CSE02198.1 | 3023 | 9594 | 244 | CSE02182.1 | 3024 | 9595 | 483 |
| 745 | I | 4 | CRZ49596.1 | 3026 | 9597 | 687 | CRZ49592.1 | 3027 | 9598 | 244 | CRZ49589.1 | 3028 | 9599 | 483 |
| 746 | I | 4 | CSC16355.1 | 3030 | 9601 | 687 | CSC16391.1 | 3031 | 9602 | 244 | CSC16431.1 | 3032 | 9603 | 483 |
| 747 | I | 4 | CSB69082.1 | 3034 | 9605 | 584 | CSB69144.1 | 3035 | 9606 | 244 | CSB69175.1 | 3036 | 9607 | 483 |
| 748 | I | 4 | CSA28373.1 | 3038 | 9609 | 687 | CSA28411.1 | 3039 | 9610 | 244 | CSA28460.1 | 3040 | 9611 | 483 |
| 749 | I | 4 | CSB84212.1 | 3042 | 9613 | 683 | CSB84179.1 | 3043 | 9614 | 244 | CSB84126.1 | 3044 | 9615 | 166 |
| 750 | I | 4 | CSB63035.1 | 3046 | 9617 | 706 | CSB63063.1 | 3047 | 9618 | 244 | CSB63093.1 | 3048 | 9619 | 483 |
| 751 | I | 4 | CSA19108.1 | 3050 | 9621 | 687 | CSA19143.1 | 3051 | 9622 | 244 | CSA19174.1 | 3052 | 9623 | 483 |
| 752 | I | 4 | CSD58035.1 | 3054 | 9625 | 687 | CSD58068.1 | 3055 | 9626 | 244 | CSD58113.1 | 3056 | 9627 | 483 |
| 753 | I | 3 | CSB77636.1 | 3058 | 9629 | 706 | CSB77620.1 | 3059 | 9630 | 244 | CSB77591.1 | 3060 | 9631 | 265 |
| 754 | I | 4 | CSB47699.1 | 3061 | 9632 | 706 | CSB47717.1 | 3062 | 9633 | 244 | CSB47737.1 | 3063 | 9634 | 483 |
| 755 | I | 4 | CSA30864.1 | 3065 | 9636 | 687 | CSA30887.1 | 3066 | 9637 | 244 | CSA30916.1 | 3067 | 9638 | 483 |
| 756 | I | 4 | CSB56036.1 | 3069 | 9640 | 687 | CSB56072.1 | 3070 | 9641 | 244 | CSB56096.1 | 3071 | 9642 | 483 |
| 757 | I | 4 | CRZ98430.1 | 3073 | 9644 | 687 | CRZ98405.1 | 3074 | 9645 | 244 | CRZ98377.1 | 3075 | 9646 | 483 |
| 758 | I | 4 | CSA01223.1 | 3077 | 9648 | 521 | CSA01278.1 | 3078 | 9649 | 244 | CSA01305.1 | 3079 | 9650 | 174 |
| 759 | I | 4 | CSB97601.1 | 3081 | 9652 | 687 | CSB97580.1 | 3082 | 9653 | 244 | CSB97551.1 | 3083 | 9654 | 307 |
| 760 | I | 4 | CSC01831.1 | 3085 | 9656 | 687 | CSC01791.1 | 3086 | 9657 | 244 | CSC01763.1 | 3087 | 9658 | 483 |
| 761 | I | 4 | CRZ46095.1 | 3089 | 9660 | 706 | CRZ46097.1 | 3090 | 9661 | 244 | CRZ46104.1 | 3091 | 9662 | 93 |
| 762 | I | 4 | CSA07930.1 | 3093 | 9664 | 687 | CSA07897.1 | 3094 | 9665 | 244 | CSA07877.1 | 3095 | 9666 | 483 |
| 763 | I | 4 | CSC23820.1 | 3097 | 9668 | 687 | CSC23781.1 | 3098 | 9669 | 244 | CSC23752.1 | 3099 | 9670 | 483 |
| 764 | I | 3 | CSB05145.1 | 3101 | 9672 | 687 | CSB05176.1 | 3102 | 9673 | 244 | CSB05201.1 | 3103 | 9674 | 483 |
| 765 | I | 4 | CSE01515.1 | 3104 | 9675 | 687 | CSE01529.1 | 3105 | 9676 | 244 | CSE01548.1 | 3106 | 9677 | 483 |
| 766 | I | 3 | CSA08484.1 | 3108 | 9679 | 687 | CSA08454.1 | 3109 | 9680 | 244 | | | | |
| 767 | I | 4 | CRZ45176.1 | 3111 | 9682 | 687 | CRZ45172.1 | 3112 | 9683 | 244 | CRZ45168.1 | 3113 | 9684 | 483 |
| 768 | I | 4 | CSA06942.1 | 3115 | 9686 | 687 | CSA06965.1 | 3116 | 9687 | 244 | CSA06996.1 | 3117 | 9688 | 483 |
| 769 | I | 4 | CSA08863.1 | 3119 | 9690 | 687 | CSA08826.1 | 3120 | 9691 | 244 | CSA08791.1 | 3121 | 9692 | 483 |
| 770 | I | 4 | CSA15692.1 | 3123 | 9694 | 687 | CSA15667.1 | 3124 | 9695 | 244 | CSA15641.1 | 3125 | 9696 | 265 |
| 771 | I | 4 | CSA74473.1 | 3127 | 9698 | 706 | CSA74503.1 | 3128 | 9699 | 244 | CSA74529.1 | 3129 | 9700 | 166 |
| 772 | I | 3 | | | | | CSB88670.1 | 3131 | 9702 | 244 | CSB88619.1 | 3132 | 9703 | 333 |
| 773 | I | 4 | CSC80217.1 | 3134 | 9705 | 687 | CSC80194.1 | 3135 | 9706 | 244 | CSC80172.1 | 3136 | 9707 | 483 |
| 774 | I | 4 | CSB83682.1 | 3138 | 9709 | 687 | CSB83710.1 | 3139 | 9710 | 244 | CSB83780.1 | 3140 | 9711 | 483 |
| 775 | I | 3 | | | | | CSB56503.1 | 3142 | 9713 | 244 | CSB56524.1 | 3143 | 9714 | 483 |
| 776 | I | 4 | CSC85490.1 | 3145 | 9716 | 687 | CSC85465.1 | 3146 | 9717 | 244 | CSC85445.1 | 3147 | 9718 | 483 |
| 777 | I | 4 | CSB86358.1 | 3149 | 9720 | 706 | CSB86328.1 | 3150 | 9721 | 244 | CSB86302.1 | 3151 | 9722 | 265 |
| 778 | I | 4 | CSD13052.1 | 3153 | 9724 | 687 | CSD13079.1 | 3154 | 9725 | 244 | CSD13105.1 | 3155 | 9726 | 483 |
| 779 | I | 4 | CSD79475.1 | 3157 | 9728 | 687 | CSD79446.1 | 3158 | 9729 | 244 | CSD79419.1 | 3159 | 9730 | 483 |
| 780 | I | 4 | CSD86966.1 | 3161 | 9732 | 687 | CSD86944.1 | 3162 | 9733 | 244 | CSD86920.1 | 3163 | 9734 | 483 |
| 781 | I | 4 | CSA39609.1 | 3165 | 9736 | 687 | CSA39643.1 | 3166 | 9737 | 244 | CSA39681.1 | 3167 | 9738 | 483 |
| 782 | I | 4 | CSC76746.1 | 3169 | 9740 | 687 | CSC76722.1 | 3170 | 9741 | 244 | CSC76697.1 | 3171 | 9742 | 483 |
| 783 | I | 4 | CSA42744.1 | 3173 | 9744 | 687 | CSA42770.1 | 3174 | 9745 | 244 | CSA42798.1 | 3175 | 9746 | 483 |
| 784 | I | 4 | CSA52054.1 | 3177 | 9748 | 687 | CSA52090.1 | 3178 | 9749 | 244 | CSA52135.1 | 3179 | 9750 | 483 |
| 785 | I | 4 | CSD31909.1 | 3181 | 9752 | 687 | CSD31884.1 | 3182 | 9753 | 244 | CSD31863.1 | 3183 | 9754 | 483 |
| 786 | I | 4 | CSD17795.1 | 3185 | 9756 | 687 | CSD17820.1 | 3186 | 9757 | 244 | CSD17844.1 | 3187 | 9758 | 483 |
| 787 | I | 4 | CSD38760.1 | 3189 | 9760 | 687 | CSD38739.1 | 3190 | 9761 | 244 | CSD38713.1 | 3191 | 9762 | 483 |
| 788 | I | 4 | CSA26418.1 | 3193 | 9764 | 687 | CSA26383.1 | 3194 | 9765 | 244 | CSA26354.1 | 3195 | 9766 | 483 |
| 789 | I | 4 | CSA45804.1 | 3197 | 9768 | 706 | CSA45841.1 | 3198 | 9769 | 244 | CSA45878.1 | 3199 | 9770 | 483 |
| 790 | I | 4 | CSD96243.1 | 3201 | 9772 | 687 | CSD96273.1 | 3202 | 9773 | 244 | CSD96300.1 | 3203 | 9774 | 483 |
| 791 | I | 4 | CSA54123.1 | 3205 | 9776 | 687 | CSA54080.1 | 3206 | 9777 | 244 | CSA54039.1 | 3207 | 9778 | 483 |
| 792 | I | 3 | CSC37722.1 | 3209 | 9780 | 443 | CSC37773.1 | 3210 | 9781 | 244 | | | | |
| 793 | I | 4 | CSA88061.1 | 3212 | 9783 | 443 | CSA88093.1 | 3213 | 9784 | 244 | CSA88149.1 | 3214 | 9785 | 350 |
| 794 | I | 4 | CSA15119.1 | 3216 | 9787 | 706 | CSA15151.1 | 3217 | 9788 | 244 | CSA15173.1 | 3218 | 9789 | 107 |
| 795 | I | 3 | | | | | CSC98411.1 | 3220 | 9791 | 218 | CSC98455.1 | 3221 | 9792 | 199 |
| 796 | I | 4 | CRZ48558.1 | 3223 | 9794 | 687 | CRZ48557.1 | 3224 | 9795 | 244 | CRZ48553.1 | 3225 | 9796 | 307 |
| 797 | I | 3 | | | | | CSB25008.1 | 3227 | 9798 | 244 | CSB25021.1 | 3228 | 9799 | 360 |
| 798 | I | 4 | CSD98728.1 | 3230 | 9801 | 687 | CSD98712.1 | 3231 | 9802 | 244 | CSD98693.1 | 3232 | 9803 | 483 |
| 799 | I | 3 | | | | | CSA36905.1 | 3234 | 9805 | 244 | CSA36950.1 | 3235 | 9806 | 231 |
| 800 | I | 3 | CSA30693.1 | 3237 | 9808 | 706 | CSA30747.1 | 3238 | 9809 | 244 | CSA30798.1 | 3239 | 9810 | 483 |
| 801 | I | 4 | CSD70399.1 | 3240 | 9811 | 687 | CSD70430.1 | 3241 | 9812 | 244 | CSD70457.1 | 3242 | 9813 | 483 |
| 802 | I | 3 | | | | | CSD13971.1 | 3244 | 9815 | 244 | CSD13924.1 | 3245 | 9816 | 483 |
| 803 | I | 4 | CSD31814.1 | 3247 | 9818 | 687 | CSD31841.1 | 3248 | 9819 | 244 | CSD31862.1 | 3249 | 9820 | 483 |
| 804 | I | 4 | CSE18852.1 | 3251 | 9822 | 687 | CSE18836.1 | 3252 | 9823 | 244 | CSE18821.1 | 3253 | 9824 | 483 |
| 805 | I | 4 | CSD93125.1 | 3255 | 9826 | 687 | CSD93151.1 | 3256 | 9827 | 244 | CSD93175.1 | 3257 | 9828 | 483 |
| 806 | I | 4 | CSA21244.1 | 3259 | 9830 | 687 | CSA21199.1 | 3260 | 9831 | 244 | CSA21173.1 | 3261 | 9832 | 483 |
| 807 | I | 4 | CSA44863.1 | 3263 | 9834 | 715 | CSA44812.1 | 3264 | 9835 | 244 | CSA44762.1 | 3265 | 9836 | 265 |
| 808 | I | 4 | CSD30711.1 | 3267 | 9838 | 687 | CSD30681.1 | 3268 | 9839 | 244 | CSD30657.1 | 3269 | 9840 | 483 |
| 809 | I | 4 | CSD92444.1 | 3271 | 9842 | 687 | CSD92423.1 | 3272 | 9843 | 244 | CSD92407.1 | 3273 | 9844 | 483 |
| 810 | I | 4 | CSD99240.1 | 3275 | 9846 | 687 | CSD99267.1 | 3276 | 9847 | 244 | CSD99291.1 | 3277 | 9848 | 483 |
| 811 | I | 4 | CSA23088.1 | 3279 | 9850 | 687 | CSA23044.1 | 3280 | 9851 | 244 | CSA23002.1 | 3281 | 9852 | 483 |
| 812 | I | 4 | CSA49073.1 | 3283 | 9854 | 706 | CSA49123.1 | 3284 | 9855 | 244 | CSA49171.1 | 3285 | 9856 | 483 |
| 813 | I | 4 | CSC63545.1 | 3287 | 9858 | 478 | CSC63565.1 | 3288 | 9859 | 244 | CSC63589.1 | 3289 | 9860 | 134 |
| 814 | I | 4 | CRZ49242.1 | 3291 | 9862 | 687 | CRZ49246.1 | 3292 | 9863 | 244 | CRZ49252.1 | 3293 | 9864 | 483 |
| 815 | I | 4 | CSC03222.1 | 3295 | 9866 | 706 | CSC03177.1 | 3296 | 9867 | 244 | CSC03135.1 | 3297 | 9868 | 483 |
| 816 | I | 4 | CSD87325.1 | 3299 | 9870 | 687 | CSD87301.1 | 3300 | 9871 | 244 | CSD87274.1 | 3301 | 9872 | 483 |
| 817 | I | 4 | CSD21169.1 | 3303 | 9874 | 687 | CSD21190.1 | 3304 | 9875 | 244 | CSD21226.1 | 3305 | 9876 | 483 |
| 818 | I | 3 | CSC95321.1 | 3307 | 9878 | 687 | CSC95342.1 | 3308 | 9879 | 244 | CSC95365.1 | 3309 | 9880 | 492 |
| 819 | I | 4 | CSA26886.1 | 3310 | 9881 | 687 | CSA26825.1 | 3311 | 9882 | 244 | CSA26777.1 | 3312 | 9883 | 166 |
| 820 | I | 4 | CSB50406.1 | 3314 | 9885 | 687 | CSB50386.1 | 3315 | 9886 | 244 | CSB50354.1 | 3316 | 9887 | 483 |
| 821 | I | 4 | CSB24741.1 | 3318 | 9889 | 599 | CSB24761.1 | 3319 | 9890 | 244 | CSB24790.1 | 3320 | 9891 | 166 |
| 822 | I | 4 | CSB68001.1 | 3322 | 9893 | 687 | CSB68025.1 | 3323 | 9894 | 244 | CSB68050.1 | 3324 | 9895 | 483 |
| 823 | I | 4 | CSB16341.1 | 3326 | 9897 | 706 | CSB16315.1 | 3327 | 9898 | 244 | CSB16293.1 | 3328 | 9899 | 483 |
| 824 | I | 4 | CRZ89319.1 | 3330 | 9901 | 687 | CRZ89282.1 | 3331 | 9902 | 244 | CRZ89245.1 | 3332 | 9903 | 483 |
| 825 | I | 4 | CSD99897.1 | 3334 | 9905 | 687 | CSD99912.1 | 3335 | 9906 | 244 | CSD99928.1 | 3336 | 9907 | 483 |
| 826 | I | 4 | CSA40795.1 | 3338 | 9909 | 687 | CSA40831.1 | 3339 | 9910 | 244 | CSA40854.1 | 3340 | 9911 | 483 |
| 827 | I | 4 | CSC19419.1 | 3342 | 9913 | 687 | CSC19396.1 | 3343 | 9914 | 244 | CSC19370.1 | 3344 | 9915 | 483 |
| 828 | I | 4 | CSD27220.1 | 3346 | 9917 | 687 | CSD27192.1 | 3347 | 9918 | 244 | CSD27166.1 | 3348 | 9919 | 483 |
| 829 | I | 4 | CSA25112.1 | 3350 | 9921 | 687 | CSA25084.1 | 3351 | 9922 | 244 | CSA25048.1 | 3352 | 9923 | 483 |
| 830 | I | 4 | CSD33112.1 | 3354 | 9925 | 531 | CSD33079.1 | 3355 | 9926 | 244 | CSD33047.1 | 3356 | 9927 | 483 |
| 831 | I | 4 | CSD26002.1 | 3358 | 9929 | 687 | CSD25975.1 | 3359 | 9930 | 244 | CSD25966.1 | 3360 | 9931 | 483 |
| 832 | I | 4 | CSD38021.1 | 3362 | 9933 | 687 | CSD37998.1 | 3363 | 9934 | 244 | CSD37970.1 | 3364 | 9935 | 483 |
| 833 | I | 4 | CSD14687.1 | 3366 | 9937 | 687 | CSD14704.1 | 3367 | 9938 | 244 | CSD14728.1 | 3368 | 9939 | 483 |
| 834 | I | 3 | CSA18679.1 | 3370 | 9941 | 687 | CSA18640.1 | 3371 | 9942 | 244 | CSA18601.1 | 3372 | 9943 | 483 |
| 835 | I | 4 | CSD46955.1 | 3373 | 9944 | 687 | CSD46995.1 | 3374 | 9945 | 244 | CSD47023.1 | 3375 | 9946 | 483 |
| 836 | I | 4 | CRZ80473.1 | 3377 | 9948 | 716 | CRZ80451.1 | 3378 | 9949 | 244 | CRZ80418.1 | 3379 | 9950 | 483 |
| 837 | I | 4 | CSC19266.1 | 3381 | 9952 | 687 | CSC19248.1 | 3382 | 9953 | 244 | CSC19228.1 | 3383 | 9954 | 483 |
| 838 | I | 4 | CSA24075.1 | 3385 | 9956 | 687 | CSA24040.1 | 3386 | 9957 | 244 | CSA24013.1 | 3387 | 9958 | 483 |
| 839 | I | 4 | CSA07992.1 | 3389 | 9960 | 687 | CSA08026.1 | 3390 | 9961 | 244 | CSA08045.1 | 3391 | 9962 | 483 |
| 840 | I | 4 | CSB55819.1 | 3393 | 9964 | 578 | CSB55765.1 | 3394 | 9965 | 244 | CSB55741.1 | 3395 | 9966 | 483 |
| 841 | I | 4 | CSA25748.1 | 3397 | 9968 | 687 | CSA25793.1 | 3398 | 9969 | 244 | CSA25829.1 | 3399 | 9970 | 483 |
| 842 | I | 4 | CSD34768.1 | 3401 | 9972 | 687 | CSD34731.1 | 3402 | 9973 | 244 | CSD34701.1 | 3403 | 9974 | 483 |
| 843 | I | 4 | CSC58199.1 | 3405 | 9976 | 571 | CSC58139.1 | 3406 | 9977 | 244 | CSC58133.1 | 3407 | 9978 | 483 |
| 844 | I | 4 | CSA23793.1 | 3409 | 9980 | 706 | CSA23829.1 | 3410 | 9981 | 244 | CSA23867.1 | 3411 | 9982 | 483 |
| 845 | I | 4 | CSB25867.1 | 3413 | 9984 | 605 | CSB25856.1 | 3414 | 9985 | 244 | CSB25844.1 | 3415 | 9986 | 307 |
| 846 | I | 4 | CSD08489.1 | 3417 | 9988 | 687 | CSD08470.1 | 3418 | 9989 | 244 | CSD08448.1 | 3419 | 9990 | 483 |
| 847 | I | 4 | CSD25549.1 | 3421 | 9992 | 687 | CSD25570.1 | 3422 | 9993 | 244 | CSD25590.1 | 3423 | 9994 | 483 |
| 848 | I | 4 | CSD28514.1 | 3425 | 9996 | 687 | CSD28543.1 | 3426 | 9997 | 244 | CSD28572.1 | 3427 | 9998 | 483 |
| 849 | I | 4 | CSA45340.1 | 3429 | 10000 | 687 | CSA45303.1 | 3430 | 10001 | 244 | CSA45263.1 | 3431 | 10002 | 483 |
| 850 | I | 4 | CSD82595.1 | 3433 | 10004 | 687 | CSD82566.1 | 3434 | 10005 | 244 | CSD82541.1 | 3435 | 10006 | 483 |
| 851 | I | 4 | CSC20854.1 | 3437 | 10008 | 687 | CSC20817.1 | 3438 | 10009 | 244 | CSC20782.1 | 3439 | 10010 | 483 |
| 852 | I | 4 | CSC43426.1 | 3441 | 10012 | 712 | CSC43406.1 | 3442 | 10013 | 244 | CSC43375.1 | 3443 | 10014 | 483 |
| 853 | I | 4 | CSC06846.1 | 3445 | 10016 | 706 | CSC06871.1 | 3446 | 10017 | 244 | CSC06894.1 | 3447 | 10018 | 483 |
| 854 | I | 4 | CSB50546.1 | 3449 | 10020 | 706 | CSB50514.1 | 3450 | 10021 | 244 | CSB50503.1 | 3451 | 10022 | 483 |
| 855 | I | 4 | CSE01188.1 | 3453 | 10024 | 687 | CSE01165.1 | 3454 | 10025 | 244 | CSE01149.1 | 3455 | 10026 | 483 |
| 856 | I | 4 | CSB99258.1 | 3457 | 10028 | 687 | CSB99303.1 | 3458 | 10029 | 244 | CSB99336.1 | 3459 | 10030 | 483 |
| 857 | I | 3 | | | | | KOY92951.1 | 3461 | 10032 | 244 | KOY92950.1 | 3462 | 10033 | 483 |
| 858 | I | 4 | KOY92344.1 | 3464 | 10035 | 706 | KOY92343.1 | 3465 | 10036 | 244 | KOY92342.1 | 3466 | 10037 | 483 |
| 859 | I | 4 | KOY97240.1 | 3468 | 10039 | 706 | KOY97239.1 | 3469 | 10040 | 244 | KOY97238.1 | 3470 | 10041 | 483 |
| 860 | I | 4 | KOZ01032.1 | 3472 | 10043 | 706 | KOZ01031.1 | 3473 | 10044 | 244 | KOZ01030.1 | 3474 | 10045 | 483 |
| 861 | I | 4 | KPA03327.1 | 3476 | 10047 | 706 | KPA03326.1 | 3477 | 10048 | 244 | KPA03325.1 | 3478 | 10049 | 483 |
| 862 | I | 4 | ALJ63816.1 | 3480 | 10051 | 706 | ALJ63817.1 | 3481 | 10052 | 244 | ALJ63818.1 | 3482 | 10053 | 483 |
| 863 | I | 4 | KTL56325.1 | 3484 | 10055 | 706 | KTL56324.1 | 3485 | 10056 | 244 | KTL56323.1 | 3486 | 10057 | 483 |
| 864 | I | 3 | | | | | KUO35497.1 | 3488 | 10059 | 244 | KUO35496.1 | 3489 | 10060 | 483 |
| 865 | I | 3 | KUP87193.1 | 3491 | 10062 | 706 | KUP87194.1 | 3492 | 10063 | 244 | KUP87195.1 | 3493 | 10064 | 483 |
| 866 | I | 4 | KUP86906.1 | 3494 | 10065 | 706 | KUP86907.1 | 3495 | 10066 | 244 | KUP86908.1 | 3496 | 10067 | 483 |
| 867 | I | 4 | KUY44285.1 | 3498 | 10069 | 706 | KUY44286.1 | 3499 | 10070 | 244 | KUY44287.1 | 3500 | 10071 | 483 |
| 868 | I | 4 | KVI44905.1 | 3502 | 10073 | 706 | KVI44904.1 | 3503 | 10074 | 244 | KVI44903.1 | 3504 | 10075 | 483 |
| 869 | I | 4 | KWW48033.1 | 3506 | 10077 | 706 | KWW48034.1 | 3507 | 10078 | 244 | KWW48035.1 | 3508 | 10079 | 483 |
| 870 | I | 4 | EIG23809.1 | 3510 | 10081 | 728 | EIG23861.1 | 3511 | 10082 | 263 | EIG23836.1 | 3512 | 10083 | 507 |
| 871 | I | 4 | KGQ46272.1 | 3514 | 10085 | 677 | KGQ46273.1 | 3515 | 10086 | 298 | KGQ46274.1 | 3516 | 10087 | 479 |
| 872 | I | 4 | KGQ51096.1 | 3518 | 10089 | 677 | KGQ51097.1 | 3519 | 10090 | 298 | KGQ51098.1 | 3520 | 10091 | 479 |
| 873 | I | 4 | KGQ65037.1 | 3522 | 10093 | 677 | KGQ65036.1 | 3523 | 10094 | 298 | KGQ65035.1 | 3524 | 10095 | 479 |
| 874 | I | 4 | ACX76639.1 | 3526 | 10097 | 584 | ACX76638.1 | 3527 | 10098 | 243 | ACX76637.1 | 3528 | 10099 | 455 |
| 875 | I | 4 | ADL24842.1 | 3530 | 10101 | 584 | ADL27028.1 | 3531 | 10102 | 243 | ADL26078.1 | 3532 | 10103 | 455 |
| 876 | I | 4 | EEO43106.1 | 3534 | 10105 | 535 | EEO43107.2 | 3535 | 10106 | 207 | EEO43108.1 | 3536 | 10107 | 402 |
| 877 | I | 4 | EPC08247.1 | 3538 | 10109 | 535 | EPC08246.1 | 3539 | 10110 | 207 | EPC08245.1 | 3540 | 10111 | 402 |
| 878 | I | 4 | ERT42791.1 | 3542 | 10113 | 583 | ERT42790.1 | 3543 | 10114 | 207 | ERT42789.1 | 3544 | 10115 | 402 |
| 879 | I | 4 | KGE62641.1 | 3546 | 10117 | 583 | KGE62642.1 | 3547 | 10118 | 232 | KGE62643.1 | 3548 | 10119 | 401 |
| 880 | I | 4 | EFE87094.1 | 3550 | 10121 | 583 | EFE87095.1 | 3551 | 10122 | 232 | EFE87096.1 | 3552 | 10123 | 402 |
| 881 | I | 4 | ABM29947.1 | 3554 | 10125 | 677 | ABM29948.1 | 3555 | 10126 | 259 | ABM29949.1 | 3556 | 10127 | 455 |
| 882 | I | 4 | ADB47624.1 | 3558 | 10129 | 616 | ADB47623.1 | 3559 | 10130 | 235 | ADB47622.1 | 3560 | 10131 | 467 |
| 883 | I | 4 | ADE85046.1 | 3562 | 10133 | 681 | ADE85047.1 | 3563 | 10134 | 334 | ADE85048.1 | 3564 | 10135 | 444 |
| 884 | I | 4 | ETD02074.1 | 3566 | 10137 | 681 | ETD02075.1 | 3567 | 10138 | 334 | ETD02172.1 | 3568 | 10139 | 431 |
| 885 | I | 4 | ETD86671.1 | 3570 | 10141 | 681 | ETD86670.1 | 3571 | 10142 | 334 | ETD86702.1 | 3572 | 10143 | 444 |
| 886 | I | 4 | ETD77748.1 | 3574 | 10145 | 681 | ETD77749.1 | 3575 | 10146 | 334 | ETD77862.1 | 3576 | 10147 | 431 |
| 887 | I | 4 | ETE54106.1 | 3578 | 10149 | 681 | ETE54107.1 | 3579 | 10150 | 334 | ETE54205.1 | 3580 | 10151 | 431 |
| 888 | I | 3 | KKI99610.1 | 3582 | 10153 | 673 | KKI99609.1 | 3583 | 10154 | 248 | KKI99608.1 | 3584 | 10155 | 488 |
| 889 | I | 4 | KFI23089.1 | 3585 | 10156 | 701 | KFI23090.1 | 3586 | 10157 | 250 | KFI23091.1 | 3587 | 10158 | 476 |
| 890 | I | 4 | KMY56660.1 | 3589 | 10160 | 648 | KMY56661.1 | 3590 | 10161 | 223 | KMY56662.1 | 3591 | 10162 | 426 |
| 891 | I | 4 | KMY64903.1 | 3593 | 10164 | 648 | KMY64904.1 | 3594 | 10165 | 223 | KMY64905.1 | 3595 | 10166 | 426 |
| 892 | I | 4 | KYD35338.1 | 3597 | 10168 | 648 | KYD35337.1 | 3598 | 10169 | 223 | KYD35336.1 | 3599 | 10170 | 426 |
| 893 | I | 4 | GAD13408.1 | 3601 | 10172 | 460 | GAD13407.1 | 3602 | 10173 | 211 | GAD13406.1 | 3603 | 10174 | 426 |
| 894 | I | 3 | EMT39963.1 | 3605 | 10176 | 540 | EMT40032.1 | 3606 | 10177 | 230 | | | | |
| 895 | I | 3 | EGO65557.1 | 3608 | 10179 | 743 | EGO65558.1 | 3609 | 10180 | 232 | EGO65559.1 | 3610 | 10181 | 476 |
| 896 | I | 3 | EKF49049.1 | 3611 | 10182 | 411 | EKF49048.1 | 3612 | 10183 | 218 | | | | |
| 897 | I | 4 | BAL95138.1 | 3614 | 10185 | 667 | BAL95139.1 | 3615 | 10186 | 208 | BAL95140.1 | 3616 | 10187 | 565 |
| 898 | I | 4 | KTC75261.1 | 3618 | 10189 | 636 | KTC75262.1 | 3619 | 10190 | 234 | KTC75263.1 | 3620 | 10191 | 482 |
| 899 | I | 4 | EFF49971.1 | 3622 | 10193 | 667 | EFF49970.1 | 3623 | 10194 | 231 | EFF49969.1 | 3624 | 10195 | 616 |
| 900 | I | 4 | ABU75897.1 | 3626 | 10197 | 729 | ABU75896.1 | 3627 | 10198 | 246 | ABU75895.1 | 3628 | 10199 | 562 |
| 901 | I | 3 | | | | | EGL73326.1 | 3630 | 10201 | 246 | EGL73325.1 | 3631 | 10202 | 523 |
| 902 | I | 3 | CCK08186.1 | 3633 | 10204 | 259 | CCK08187.1 | 3634 | 10205 | 246 | CCK08188.1 | 3635 | 10206 | 562 |
| 903 | I | 4 | CCK02754.1 | 3636 | 10207 | 656 | CCK02755.1 | 3637 | 10208 | 246 | CCK02756.1 | 3638 | 10209 | 523 |
| 904 | I | 4 | CCO48375.1 | 3640 | 10211 | 706 | CCO48376.1 | 3641 | 10212 | 244 | CCO48377.1 | 3642 | 10213 | 482 |
| 905 | I | 4 | CCK80596.1 | 3644 | 10215 | 641 | CCK80597.1 | 3645 | 10216 | 250 | CCK80598.1 | 3646 | 10217 | 485 |
| 906 | I | 4 | KGD34430.1 | 3648 | 10219 | 667 | KGD34483.1 | 3649 | 10220 | 234 | KGD34420.1 | 3650 | 10221 | 573 |
| 907 | I | 4 | KGS74771.1 | 3652 | 10223 | 670 | KGS74845.1 | 3653 | 10224 | 241 | KGS74775.1 | 3654 | 10225 | 546 |
| 908 | I | 4 | KGW09067.1 | 3656 | 10227 | 667 | KGW09406.1 | 3657 | 10228 | 239 | KGW09097.1 | 3658 | 10229 | 454 |
| 909 | I | 4 | KGX47863.1 | 3660 | 10231 | 555 | KGX47852.1 | 3661 | 10232 | 234 | KGX47883.1 | 3662 | 10233 | 478 |
| 910 | I | 4 | KGX39518.1 | 3664 | 10235 | 667 | KGX39502.1 | 3665 | 10236 | 234 | KGX39650.1 | 3666 | 10237 | 573 |
| 911 | I | 4 | CPH56696.1 | 3668 | 10239 | 780 | CPH56659.1 | 3669 | 10240 | 241 | CPH56628.1 | 3670 | 10241 | 605 |
| 912 | I | 4 | KSB52477.1 | 3672 | 10243 | 729 | KSB52476.1 | 3673 | 10244 | 246 | KSB52475.1 | 3674 | 10245 | 560 |
| 913 | I | 3 | AEF17805.1 | 3676 | 10247 | 540 | AEF17804.1 | 3677 | 10248 | 230 | | | | |
| 914 | I | 4 | ENW17902.1 | 3679 | 10250 | 146 | ENW17901.1 | 3680 | 10251 | 243 | ENW17900.1 | 3681 | 10252 | 474 |
| 915 | I | 4 | ABO66647.1 | 3683 | 10254 | 626 | ABO66648.1 | 3684 | 10255 | 223 | ABO66649.1 | 3685 | 10256 | 426 |
| 916 | I | 3 | ACI99727.1 | 3687 | 10258 | 763 | ACI99728.1 | 3688 | 10259 | 314 | ACI99729.1 | 3689 | 10260 | 484 |
| 917 | I | 4 | ACS21194.1 | 3690 | 10261 | 667 | ACS21193.1 | 3691 | 10262 | 234 | ACS21192.1 | 3692 | 10263 | 574 |
| 918 | I | 3 | EIC29071.1 | 3694 | 10265 | 605 | EIC29072.1 | 3695 | 10266 | 231 | EIC29073.1 | 3696 | 10267 | 527 |
| 919 | I | 4 | EET81943.1 | 3697 | 10268 | 686 | EET81942.1 | 3698 | 10269 | 243 | EET81941.1 | 3699 | 10270 | 471 |
| 920 | I | 4 | ENU31377.1 | 3701 | 10272 | 300 | ENU31376.1 | 3702 | 10273 | 243 | ENU31375.1 | 3703 | 10274 | 456 |
| 921 | I | 4 | ALA59739.1 | 3705 | 10276 | 656 | ALA59738.1 | 3706 | 10277 | 240 | ALA59737.1 | 3707 | 10278 | 548 |
| 922 | I | 4 | AGB41055.1 | 3709 | 10280 | 652 | AGB41056.1 | 3710 | 10281 | 228 | AGB41057.1 | 3711 | 10282 | 417 |
| 923 | I | 4 | AEI93759.1 | 3713 | 10284 | 462 | AEI93760.1 | 3714 | 10285 | 323 | AEI93761.1 | 3715 | 10286 | 437 |
| 924 | I | 4 | CCU78667.1 | 3717 | 10288 | 654 | CCU78668.1 | 3718 | 10289 | 262 | CCU78669.1 | 3719 | 10290 | 398 |
| 925 | I | 4 | ERG17892.1 | 3721 | 10292 | 691 | ERG17893.1 | 3722 | 10293 | 247 | ERG17894.1 | 3723 | 10294 | 480 |
| 926 | I | 4 | ALF60586.1 | 3725 | 10296 | 688 | ALF60587.1 | 3726 | 10297 | 243 | ALF60588.1 | 3727 | 10298 | 457 |
| 927 | I | 3 | AEM78245.1 | 3729 | 10300 | 540 | AEM78246.1 | 3730 | 10301 | 230 | | | | |
| 928 | I | 4 | EFV93687.1 | 3732 | 10303 | 690 | EFV93688.1 | 3733 | 10304 | 236 | EFV93689.1 | 3734 | 10305 | 565 |
| 929 | I | 4 | KRI14520.1 | 3736 | 10307 | 688 | KRI14521.1 | 3737 | 10308 | 243 | KRI14522.1 | 3738 | 10309 | 457 |
| 930 | I | 4 | AEJ38288.1 | 3740 | 10311 | 647 | AEJ38287.1 | 3741 | 10312 | 210 | AEJ38286.1 | 3742 | 10313 | 398 |
| 931 | I | 4 | AEW03630.1 | 3744 | 10315 | 647 | AEW03629.1 | 3745 | 10316 | 229 | AEW03628.1 | 3746 | 10317 | 398 |
| 932 | I | 4 | KFX14697.1 | 3748 | 10319 | 729 | KFX14696.1 | 3749 | 10320 | 245 | KFX14695.1 | 3750 | 10321 | 560 |
| 933 | I | 4 | KKW93255.1 | 3752 | 10323 | 641 | KKW93256.1 | 3753 | 10324 | 242 | KKW94108.1 | 3754 | 10325 | 426 |
| 934 | I | 4 | KVX03061.1 | 3756 | 10327 | 723 | KVX03060.1 | 3757 | 10328 | 252 | KVX03059.1 | 3758 | 10329 | 565 |
| 935 | I | 4 | KJM54480.1 | 3760 | 10331 | 851 | KJM54479.1 | 3761 | 10332 | 245 | KJM54478.1 | 3762 | 10333 | 597 |
| 936 | I | 4 | KLF88330.1 | 3764 | 10335 | 729 | KLF88329.1 | 3765 | 10336 | 246 | KLF88328.1 | 3766 | 10337 | 560 |
| 937 | I | 4 | KLG16016.1 | 3768 | 10339 | 729 | KLG16015.1 | 3769 | 10340 | 246 | KLG16014.1 | 3770 | 10341 | 560 |
| 938 | I | 4 | KLP43536.1 | 3772 | 10343 | 729 | KLP43535.1 | 3773 | 10344 | 246 | KLP43534.1 | 3774 | 10345 | 560 |
| 939 | I | 4 | KTK05050.1 | 3776 | 10347 | 851 | KTK05049.1 | 3777 | 10348 | 245 | KTK05048.1 | 3778 | 10349 | 597 |
| 940 | I | 4 | KTJ30242.1 | 3780 | 10351 | 729 | KTJ30243.1 | 3781 | 10352 | 246 | KTJ30244.1 | 3782 | 10353 | 560 |
| 941 | I | 4 | KTI23428.1 | 3784 | 10355 | 851 | KTI23427.1 | 3785 | 10356 | 245 | KTI23426.1 | 3786 | 10357 | 597 |
| 942 | I | 4 | AKL12331.1 | 3788 | 10359 | 729 | AKL12332.1 | 3789 | 10360 | 246 | AKL12333.1 | 3790 | 10361 | 560 |
| 943 | I | 3 | AFZ43911.1 | 3792 | 10363 | 746 | AFZ43910.1 | 3793 | 10364 | 236 | AFZ43909.1 | 3794 | 10365 | 507 |
| 944 | I | 3 | ACK72258.1 | 3795 | 10366 | 456 | ACK72256.1 | 3796 | 10367 | 222 | ACK72255.1 | 3797 | 10368 | 501 |
| 945 | I | 4 | AJG24952.1 | 3798 | 10369 | 667 | AJG24951.1 | 3799 | 10370 | 53 | AJG24950.1 | 3800 | 10371 | 556 |
| 946 | I | 3 | EAW43011.1 | 3802 | 10373 | 656 | EAW43010.1 | 3803 | 10374 | 216 | EAW43009.1 | 3804 | 10375 | 464 |
| 947 | I | 3 | AHJ30056.1 | 3805 | 10376 | 635 | AHJ30057.1 | 3806 | 10377 | 216 | AHJ30058.1 | 3807 | 10378 | 464 |
| 948 | I | 4 | AJE15429.1 | 3808 | 10379 | 731 | AJE15428.1 | 3809 | 10380 | 245 | AJE15427.1 | 3810 | 10381 | 558 |
| 949 | I | 3 | CEJ44798.1 | 3812 | 10383 | 627 | CEJ44797.1 | 3813 | 10384 | 214 | CEJ44796.1 | 3814 | 10385 | 459 |
| 950 | I | 4 | EHO11882.1 | 3815 | 10386 | 509 | EHO11881.1 | 3816 | 10387 | 238 | EHO11883.1 | 3817 | 10388 | 475 |
| 951 | I | 4 | CAY72975.1 | 3819 | 10390 | 729 | CAY72976.1 | 3820 | 10391 | 245 | CAY72978.1 | 3821 | 10392 | 566 |
| 952 | I | 4 | CAX54367.1 | 3823 | 10394 | 729 | CAX54368.1 | 3824 | 10395 | 245 | CAX54369.1 | 3825 | 10396 | 566 |
| 953 | I | 4 | AGA90547.1 | 3827 | 10398 | 586 | AGA90548.1 | 3828 | 10399 | 283 | AGA90549.1 | 3829 | 10400 | 425 |
| 954 | I | 4 | CUH46419.1 | 3831 | 10402 | 681 | CUH46420.1 | 3832 | 10403 | 372 | CUH46421.1 | 3833 | 10404 | 449 |
| 955 | I | 4 | AMO80628.1 | 3835 | 10406 | 729 | AMO80629.1 | 3836 | 10407 | 247 | AMO80630.1 | 3837 | 10408 | 563 |
| 956 | I | 4 | AGP47378.1 | 3839 | 10410 | 572 | AGP47379.1 | 3840 | 10411 | 263 | AGP47380.1 | 3841 | 10412 | 443 |
| 957 | I | 4 | KIN78156.1 | 3843 | 10414 | 680 | KIN78155.1 | 3844 | 10415 | 367 | KIN78154.1 | 3845 | 10416 | 738 |
| 958 | I | 4 | AMS40143.1 | 3847 | 10418 | 651 | AMS40142.1 | 3848 | 10419 | 302 | AMS40141.1 | 3849 | 10420 | 461 |
| 959 | I | 4 | ABK50003.1 | 3851 | 10422 | 696 | ABK50004.1 | 3852 | 10423 | 243 | ABK50005.1 | 3853 | 10424 | 475 |
| 960 | I | 4 | CAP40556.1 | 3855 | 10426 | 680 | CAP40555.1 | 3856 | 10427 | 237 | CAP40554.1 | 3857 | 10428 | 558 |
| 961 | I | 4 | ERI29961.1 | 3859 | 10430 | 670 | ERI29880.1 | 3860 | 10431 | 241 | ERI29995.1 | 3861 | 10432 | 546 |
| 962 | I | 4 | EPZ86961.1 | 3863 | 10434 | 670 | EPZ86615.1 | 3864 | 10435 | 241 | EPZ86681.1 | 3865 | 10436 | 546 |
| 963 | I | 4 | KKI79474.1 | 3867 | 10438 | 670 | KKI79473.1 | 3868 | 10439 | 241 | KKI79472.1 | 3869 | 10440 | 546 |
| 964 | I | 4 | KVD94073.1 | 3871 | 10442 | 674 | KVD94072.1 | 3872 | 10443 | 236 | KVD94070.1 | 3873 | 10444 | 261 |
| 965 | I | 4 | KVT83333.1 | 3875 | 10446 | 667 | KVT83334.1 | 3876 | 10447 | 234 | KVT83335.1 | 3877 | 10448 | 576 |
| 966 | I | 4 | KVW62590.1 | 3879 | 10450 | 667 | KVW62589.1 | 3880 | 10451 | 234 | KVW62588.1 | 3881 | 10452 | 576 |
| 967 | I | 3 | AFZ37310.1 | 3883 | 10454 | 781 | AFZ37311.1 | 3884 | 10455 | 264 | AFZ37312.1 | 3885 | 10456 | 502 |
| 968 | I | 3 | ADD01957.1 | 3886 | 10457 | 540 | ADD01958.1 | 3887 | 10458 | 230 | | | | |
| 969 | I | 4 | AEG43650.1 | 3889 | 10460 | 541 | AEG43649.1 | 3890 | 10461 | 277 | AEG43648.1 | 3891 | 10462 | 514 |
| 970 | I | 4 | ACY51399.1 | 3893 | 10464 | 706 | ACY51400.1 | 3894 | 10465 | 244 | ACY51401.1 | 3895 | 10466 | 483 |
| 971 | I | 4 | EDN58079.1 | 3897 | 10468 | 706 | EDN58080.1 | 3898 | 10469 | 244 | EDN58081.1 | 3899 | 10470 | 483 |
| 972 | I | 4 | AIR88232.1 | 3901 | 10472 | 730 | AIR88231.1 | 3902 | 10473 | 247 | AIR88230.1 | 3903 | 10474 | 542 |
| 973 | I | 4 | ABC30363.1 | 3905 | 10476 | 666 | ABC30364.1 | 3906 | 10477 | 263 | ABC30365.1 | 3907 | 10478 | 493 |
| 974 | I | 4 | ALV90959.1 | 3909 | 10480 | 729 | ALV90958.1 | 3910 | 10481 | 245 | ALV90957.1 | 3911 | 10482 | 560 |
| 975 | I | 4 | EDL49433.1 | 3913 | 10484 | 641 | EDL49432.1 | 3914 | 10485 | 242 | EDL49431.1 | 3915 | 10486 | 429 |
| 976 | I | 4 | CAQ68802.1 | 3917 | 10488 | 663 | CAQ68801.1 | 3918 | 10489 | 252 | CAQ68800.1 | 3919 | 10490 | 554 |
| 977 | I | 4 | BAH37227.1 | 3921 | 10492 | 669 | BAH37228.1 | 3922 | 10493 | 230 | BAH37229.1 | 3923 | 10494 | 459 |
| 978 | I | 4 | KGU86620.1 | 3925 | 10496 | 730 | KGU86621.1 | 3926 | 10497 | 247 | KGU86622.1 | 3927 | 10498 | 542 |
| 979 | I | 3 | AFE04995.1 | 3929 | 10500 | 644 | | | | | AFE04997.1 | 3930 | 10501 | 464 |
| 980 | I | 4 | EHJ60585.1 | 3932 | 10503 | 641 | EHJ60586.1 | 3933 | 10504 | 242 | EHJ60587.1 | 3934 | 10505 | 458 |
| 981 | I | 4 | AIT79637.1 | 3936 | 10507 | 641 | AIT79636.1 | 3937 | 10508 | 242 | AIT79635.1 | 3938 | 10509 | 429 |
| 982 | I | 3 | CAV18535.1 | 3940 | 10511 | 688 | CAV18536.1 | 3941 | 10512 | 218 | CAV18537.1 | 3942 | 10513 | 625 |
| 983 | I | 4 | ADP10192.1 | 3943 | 10514 | 729 | ADP10191.1 | 3944 | 10515 | 245 | ADP10190.1 | 3945 | 10516 | 566 |
| 984 | I | 4 | ABM39550.1 | 3947 | 10518 | 816 | ABM39549.1 | 3948 | 10519 | 235 | ABM39548.1 | 3949 | 10520 | 602 |
| 985 | I | 4 | EIL89340.1 | 3951 | 10522 | 631 | EIL89341.1 | 3952 | 10523 | 236 | EIL89342.1 | 3953 | 10524 | 559 |
| 986 | I | 4 | GAC31728.1 | 3955 | 10526 | 692 | GAC31729.1 | 3956 | 10527 | 243 | GAC31730.1 | 3957 | 10528 | 475 |
| 987 | I | 3 | AFZ12767.1 | 3959 | 10530 | 726 | AFZ12766.1 | 3960 | 10531 | 239 | AFZ12765.1 | 3961 | 10532 | 474 |
| 988 | I | 4 | KFK00375.1 | 3962 | 10533 | 729 | KFK00374.1 | 3963 | 10534 | 246 | KFK00373.1 | 3964 | 10535 | 560 |
| 989 | I | 4 | CDT04278.1 | 3966 | 10537 | 704 | CDT04296.1 | 3967 | 10538 | 243 | CDT04305.1 | 3968 | 10539 | 483 |
| 990 | I | 4 | CDT11699.1 | 3970 | 10541 | 704 | CDT11706.1 | 3971 | 10542 | 243 | CDT11714.1 | 3972 | 10543 | 483 |
| 991 | I | 4 | KXV35460.1 | 3974 | 10545 | 672 | KXV35459.1 | 3975 | 10546 | 287 | KXV35477.1 | 3976 | 10547 | 425 |
| 992 | I | 4 | EAT06411.1 | 3978 | 10549 | 507 | EAT06417.1 | 3979 | 10550 | 272 | EAT06418.1 | 3980 | 10551 | 486 |
| 993 | I | 4 | BAQ44674.1 | 3982 | 10553 | 679 | BAQ44675.1 | 3983 | 10554 | 310 | BAQ44676.1 | 3984 | 10555 | 438 |
| 994 | I | 4 | CCE22484.1 | 3986 | 10557 | 642 | CCE22485.1 | 3987 | 10558 | 252 | CCE22486.1 | 3988 | 10559 | 498 |
| 995 | I | 4 | CEG51215.1 | 3990 | 10561 | 781 | CEG51216.1 | 3991 | 10562 | 254 | CEG51217.1 | 3992 | 10563 | 754 |
| 996 | I | 4 | EJI89226.1 | 3994 | 10565 | 729 | EJI89227.1 | 3995 | 10566 | 247 | EJI89228.1 | 3996 | 10567 | 567 |
| 997 | I | 4 | KDE37034.1 | 3998 | 10569 | 729 | KDE37033.1 | 3999 | 10570 | 247 | KDE37032.1 | 4000 | 10571 | 569 |
| 998 | I | 3 | AFZ51166.1 | 4002 | 10573 | 481 | AFZ51165.1 | 4003 | 10574 | 240 | AFZ51164.1 | 4004 | 10575 | 501 |
| 999 | I | 3 | GAE46380.1 | 4005 | 10576 | 629 | GAE46381.1 | 4006 | 10577 | 223 | | | | |
| 1000 | I | 4 | EAQ47302.1 | 4008 | 10579 | 680 | EAQ47301.1 | 4009 | 10580 | 367 | EAQ47300.1 | 4010 | 10581 | 741 |
| 1001 | I | 4 | EDP59419.1 | 4012 | 10583 | 706 | EDP59418.1 | 4013 | 10584 | 244 | EDP59417.1 | 4014 | 10585 | 483 |
| 1002 | I | 4 | CUH52012.1 | 4016 | 10587 | 486 | CUH52011.1 | 4017 | 10588 | 358 | CUH52010.1 | 4018 | 10589 | 436 |
| 1003 | I | 4 | CVK01182.1 | 4020 | 10591 | 844 | CVK01173.1 | 4021 | 10592 | 240 | CVK01163.1 | 4022 | 10593 | 658 |
| 1004 | I | 4 | ABE74734.1 | 4024 | 10595 | 688 | ABE74735.1 | 4025 | 10596 | 243 | ABE74736.1 | 4026 | 10597 | 473 |
| 1005 | I | 3 | CCW17770.1 | 4028 | 10599 | 549 | | | | | CCW17772.1 | 4029 | 10600 | 468 |
| 1006 | I | 3 | ABM05098.1 | 4031 | 10602 | 454 | ABM05096.1 | 4032 | 10603 | 220 | ABM05095.1 | 4033 | 10604 | 541 |
| 1007 | I | 4 | AFU46476.1 | 4034 | 10605 | 727 | AFU46477.1 | 4035 | 10606 | 255 | AFU46478.1 | 4036 | 10607 | 646 |
| 1008 | I | 4 | EZI30545.1 | 4038 | 10609 | 730 | EZI30546.1 | 4039 | 10610 | 247 | EZI30547.1 | 4040 | 10611 | 542 |
| 1009 | I | 3 | ADL55134.1 | 4042 | 10613 | 648 | ADL55135.1 | 4043 | 10614 | 238 | ADL55136.1 | 4044 | 10615 | 527 |
| 1010 | I | 4 | GAA77183.1 | 4045 | 10616 | 692 | GAA77182.1 | 4046 | 10617 | 243 | GAA77181.1 | 4047 | 10618 | 476 |
| 1011 | I | 4 | AMK16850.1 | 4049 | 10620 | 660 | AMK16851.1 | 4050 | 10621 | 236 | AMK16852.1 | 4051 | 10622 | 560 |
| 1012 | I | 3 | ADQ06821.1 | 4053 | 10624 | 539 | ADQ06820.1 | 4054 | 10625 | 241 | | | | |
| 1013 | I | 4 | EFN90481.1 | 4056 | 10627 | 460 | EFN90483.1 | 4057 | 10628 | 230 | EFN90478.1 | 4058 | 10629 | 459 |
| 1014 | I | 3 | | | | | KXB76242.1 | 4060 | 10631 | 230 | KXB76241.1 | 4061 | 10632 | 459 |
| 1015 | I | 4 | EPR18405.1 | 4063 | 10634 | 669 | EPR18406.1 | 4064 | 10635 | 242 | EPR18407.1 | 4065 | 10636 | 466 |
| 1016 | I | 4 | KMO18101.1 | 4067 | 10638 | 907 | KMO18102.1 | 4068 | 10639 | 260 | KMO18103.1 | 4069 | 10640 | 465 |
| 1017 | I | 3 | KER36348.1 | 4071 | 10642 | 641 | KER36349.1 | 4072 | 10643 | 242 | KER36350.1 | 4073 | 10644 | 466 |
| 1018 | I | 4 | ACT17070.1 | 4074 | 10645 | 666 | ACT17071.1 | 4075 | 10646 | 239 | ACT17072.1 | 4076 | 10647 | 556 |
| 1019 | I | 4 | KMW37149.1 | 4078 | 10649 | 667 | KMW37148.1 | 4079 | 10650 | 231 | KMW37147.1 | 4080 | 10651 | 616 |
| 1020 | I | 3 | | | | | CUB00791.1 | 4082 | 10653 | 322 | CUB00793.1 | 4083 | 10654 | 437 |
| 1021 | I | 4 | ADJ28737.1 | 4085 | 10656 | 700 | ADJ28736.1 | 4086 | 10657 | 250 | ADJ28735.1 | 4087 | 10658 | 477 |
| 1022 | I | 3 | | | | | AJR24432.1 | 4089 | 10660 | 236 | AJR24433.1 | 4090 | 10661 | 560 |
| 1023 | I | 4 | AJR25287.1 | 4092 | 10663 | 657 | AJR25288.1 | 4093 | 10664 | 236 | AJR25289.1 | 4094 | 10665 | 557 |
| 1024 | I | 4 | EHO63928.1 | 4096 | 10667 | 584 | EHO63911.1 | 4097 | 10668 | 222 | EHO63912.1 | 4098 | 10669 | 452 |
| 1025 | I | 4 | KVM95917.1 | 4100 | 10671 | 674 | KVM95916.1 | 4101 | 10672 | 236 | KVM95915.1 | 4102 | 10673 | 475 |
| 1026 | I | 4 | EDY06977.1 | 4104 | 10675 | 648 | EDY06976.1 | 4105 | 10676 | 223 | EDY06975.1 | 4106 | 10677 | 426 |
| 1027 | I | 3 | AMD01537.1 | 4108 | 10679 | 656 | AMD01536.1 | 4109 | 10680 | 254 | AMD01535.1 | 4110 | 10681 | 570 |
| 1028 | I | 4 | EHR42621.1 | 4111 | 10682 | 745 | EHR42622.1 | 4112 | 10683 | 263 | EHR42623.1 | 4113 | 10684 | 495 |
| 1029 | I | 4 | EHD22817.1 | 4115 | 10686 | 729 | EHD22818.1 | 4116 | 10687 | 246 | EHD22819.1 | 4117 | 10688 | 560 |
| 1030 | I | 4 | CRI66748.1 | 4119 | 10690 | 660 | CRI66749.1 | 4120 | 10691 | 242 | CRI66750.1 | 4121 | 10692 | 576 |
| 1031 | I | 4 | EFY05649.1 | 4123 | 10694 | 706 | EFY05648.1 | 4124 | 10695 | 244 | EFY05647.1 | 4125 | 10696 | 461 |
| 1032 | I | 4 | EKN30947.1 | 4127 | 10698 | 667 | EKN30946.1 | 4128 | 10699 | 231 | EKN30945.1 | 4129 | 10700 | 616 |
| 1033 | I | 4 | KPL51797.1 | 4131 | 10702 | 671 | KPL51798.1 | 4132 | 10703 | 288 | KPL51799.1 | 4133 | 10704 | 467 |
| 1034 | I | 3 | AMN39053.1 | 4135 | 10706 | 688 | | | | | AMN39055.1 | 4136 | 10707 | 481 |
| 1035 | I | 4 | KMV29900.1 | 4138 | 10709 | 605 | KMV29899.1 | 4139 | 10710 | 261 | KMV29898.1 | 4140 | 10711 | 468 |
| 1036 | I | 4 | CCA91254.1 | 4142 | 10713 | 612 | CCA91255.1 | 4143 | 10714 | 258 | CCA91256.1 | 4144 | 10715 | 433 |
| 1037 | I | 4 | CCA92387.1 | 4146 | 10717 | 641 | CCA92388.1 | 4147 | 10718 | 242 | CCA92389.1 | 4148 | 10719 | 458 |
| 1038 | I | 4 | ADE38421.1 | 4150 | 10721 | 656 | ADE38422.1 | 4151 | 10722 | 270 | ADE38423.1 | 4152 | 10723 | 462 |
| 1039 | I | 4 | EPR27897.1 | 4154 | 10725 | 648 | EPR27898.1 | 4155 | 10726 | 230 | EPR27899.1 | 4156 | 10727 | 426 |
| 1040 | I | 4 | KYC33906.1 | 4158 | 10729 | 657 | KYC33907.1 | 4159 | 10730 | 236 | KYC33908.1 | 4160 | 10731 | 557 |
| 1041 | I | 3 | ADW69601.1 | 4162 | 10733 | 650 | ADW69602.1 | 4163 | 10734 | 227 | ADW69603.1 | 4164 | 10735 | 445 |
| 1042 | I | 4 | EPR43451.1 | 4165 | 10736 | 571 | EPR43452.1 | 4166 | 10737 | 255 | EPR43453.1 | 4167 | 10738 | 454 |
| 1043 | I | 3 | ESU31843.1 | 4169 | 10740 | 629 | ESU31842.1 | 4170 | 10741 | 223 | | | | |
| 1044 | I | 4 | KHJ69401.1 | 4172 | 10743 | 729 | KHJ69400.1 | 4173 | 10744 | 245 | KHJ69399.1 | 4174 | 10745 | 560 |
| 1045 | I | 4 | ERO56341.1 | 4176 | 10747 | 729 | ERO56340.1 | 4177 | 10748 | 247 | ERO56339.1 | 4178 | 10749 | 561 |
| 1046 | I | 4 | KHN63194.1 | 4180 | 10751 | 729 | KHN63195.1 | 4181 | 10752 | 247 | KHN63196.1 | 4182 | 10753 | 561 |
| 1047 | I | 3 | KKD39674.1 | 4184 | 10755 | 733 | KKD39673.1 | 4185 | 10756 | 238 | KMW70735.1 | 4186 | 10757 | 384 |
| 1048 | I | 4 | CUB04084.1 | 4187 | 10758 | 621 | CUB04083.1 | 4188 | 10759 | 252 | CUB04082.1 | 4189 | 10760 | 571 |
| 1049 | I | 4 | EJM18419.1 | 4191 | 10762 | 730 | EJM18418.1 | 4192 | 10763 | 247 | EJM18417.1 | 4193 | 10764 | 541 |
| 1050 | I | 4 | EUB73632.1 | 4195 | 10766 | 771 | EUB73631.1 | 4196 | 10767 | 247 | EUB73630.1 | 4197 | 10768 | 652 |
| 1051 | I | 3 | EIL96364.1 | 4199 | 10770 | 625 | EIL96363.1 | 4200 | 10771 | 241 | EIL96362.1 | 4201 | 10772 | 523 |
| 1052 | I | 4 | EOU44970.1 | 4202 | 10773 | 851 | EOU44971.1 | 4203 | 10774 | 244 | EOU44972.1 | 4204 | 10775 | 595 |
| 1053 | I | 4 | ELC22340.1 | 4206 | 10777 | 851 | ELC22341.1 | 4207 | 10778 | 245 | ELC22342.1 | 4208 | 10779 | 595 |
| 1054 | I | 4 | EOU82992.1 | 4210 | 10781 | 851 | EOU82993.1 | 4211 | 10782 | 244 | EOU82994.1 | 4212 | 10783 | 595 |
| 1055 | I | 4 | KXU33094.1 | 4214 | 10785 | 657 | KXU33093.1 | 4215 | 10786 | 236 | KXU33092.1 | 4216 | 10787 | 550 |
| 1056 | I | 4 | EOV44196.1 | 4218 | 10789 | 851 | EOV44197.1 | 4219 | 10790 | 245 | EOV44198.1 | 4220 | 10791 | 595 |
| 1057 | I | 4 | EOQ55527.1 | 4222 | 10793 | 729 | EOQ55528.1 | 4223 | 10794 | 247 | EOQ55529.1 | 4224 | 10795 | 560 |
| 1058 | I | 4 | EOV91297.1 | 4226 | 10797 | 851 | EOV91298.1 | 4227 | 10798 | 245 | EOV91299.1 | 4228 | 10799 | 595 |
| 1059 | I | 4 | EOW68209.1 | 4230 | 10801 | 851 | EOW68210.1 | 4231 | 10802 | 245 | EOW68211.1 | 4232 | 10803 | 595 |
| 1060 | I | 4 | AFY17876.1 | 4234 | 10805 | 800 | AFY17877.1 | 4235 | 10806 | 247 | AFY17878.1 | 4236 | 10807 | 720 |
| 1061 | I | 4 | KHK57292.1 | 4238 | 10809 | 685 | KHK57293.1 | 4239 | 10810 | 236 | KHK57294.1 | 4240 | 10811 | 558 |
| 1062 | I | 4 | ENW78275.1 | 4242 | 10813 | 686 | ENW78276.1 | 4243 | 10814 | 243 | ENW78277.1 | 4244 | 10815 | 462 |
| 1063 | I | 4 | EME68216.1 | 4246 | 10817 | 827 | EME68215.1 | 4247 | 10818 | 251 | EME68214.1 | 4248 | 10819 | 457 |
| 1064 | I | 4 | CDE94534.1 | 4250 | 10821 | 616 | CDE94533.1 | 4251 | 10822 | 235 | CDE94532.1 | 4252 | 10823 | 467 |
| 1065 | I | 4 | CDA50128.1 | 4254 | 10825 | 657 | CDA50129.1 | 4255 | 10826 | 228 | CDA50130.1 | 4256 | 10827 | 437 |
| 1066 | I | 4 | ESQ90328.1 | 4258 | 10829 | 648 | ESQ90329.1 | 4259 | 10830 | 251 | ESQ90330.1 | 4260 | 10831 | 455 |
| 1067 | I | 4 | ESQ74514.1 | 4262 | 10833 | 662 | ESQ74515.1 | 4263 | 10834 | 267 | ESQ74516.1 | 4264 | 10835 | 464 |
| 1068 | I | 4 | AII86799.1 | 4266 | 10837 | 616 | AII86800.1 | 4267 | 10838 | 431 | AII86801.1 | 4268 | 10839 | 675 |
| 1069 | I | 4 | KPQ04149.1 | 4270 | 10841 | 692 | KPQ04148.1 | 4271 | 10842 | 243 | KPQ04147.1 | 4272 | 10843 | 475 |
| 1070 | I | 4 | CUS49192.1 | 4274 | 10845 | 692 | CUS49191.1 | 4275 | 10846 | 243 | CUS49190.1 | 4276 | 10847 | 475 |
| 1071 | I | 4 | ESN27965.1 | 4278 | 10849 | 711 | ESN27964.1 | 4279 | 10850 | 245 | ESN27963.1 | 4280 | 10851 | 560 |
| 1072 | I | 4 | EPJ56201.1 | 4282 | 10853 | 678 | EPJ56200.1 | 4283 | 10854 | 350 | EPJ56199.1 | 4284 | 10855 | 552 |
| 1073 | I | 4 | EQA99142.1 | 4286 | 10857 | 652 | EQA99141.1 | 4287 | 10858 | 250 | EQA99140.1 | 4288 | 10859 | 476 |
| 1074 | I | 4 | KMS61577.1 | 4290 | 10861 | 652 | KMS61417.1 | 4291 | 10862 | 250 | KMS61578.1 | 4292 | 10863 | 476 |
| 1075 | I | 4 | AGT31732.2 | 4294 | 10865 | 650 | AGT31733.1 | 4295 | 10866 | 223 | AGT31734.1 | 4296 | 10867 | 426 |
| 1076 | I | 4 | AIY66372.1 | 4298 | 10869 | 675 | AIY65737.1 | 4299 | 10870 | 247 | AIY65736.1 | 4300 | 10871 | 480 |
| 1077 | I | 4 | KXW55388.1 | 4302 | 10873 | 653 | KXW55387.1 | 4303 | 10874 | 249 | KXW55386.1 | 4304 | 10875 | 493 |
| 1078 | I | 4 | AKU92633.1 | 4306 | 10877 | 529 | AKU92632.1 | 4307 | 10878 | 238 | AKU92631.1 | 4308 | 10879 | 470 |
| 1079 | I | 3 | ERS81916.1 | 4310 | 10881 | 678 | ERS81915.1 | 4311 | 10882 | 263 | ERS81914.1 | 4312 | 10883 | 246 |
| 1080 | I | 3 | ERS83845.1 | 4313 | 10884 | 666 | | | | | ERS83848.1 | 4314 | 10885 | 578 |
| 1081 | I | 4 | KXU87120.1 | 4316 | 10887 | 897 | KXU87121.1 | 4317 | 10888 | 241 | KXU87122.1 | 4318 | 10889 | 597 |
| 1082 | I | 4 | KLU37751.1 | 4320 | 10891 | 667 | KLU37750.1 | 4321 | 10892 | 234 | KLU37749.1 | 4322 | 10893 | 578 |
| 1083 | I | 4 | EUC70211.1 | 4324 | 10895 | 632 | EUC70007.1 | 4325 | 10896 | 251 | EUC70008.1 | 4326 | 10897 | 508 |
| 1084 | I | 4 | EXI82583.1 | 4328 | 10899 | 679 | EXI82584.1 | 4329 | 10900 | 236 | EXI82585.1 | 4330 | 10901 | 573 |
| 1085 | I | 4 | KAK42943.1 | 4332 | 10903 | 785 | KAK42944.1 | 4333 | 10904 | 252 | KAK42945.1 | 4334 | 10905 | 618 |
| 1086 | I | 4 | BAO84440.1 | 4336 | 10907 | 644 | BAO84441.1 | 4337 | 10908 | 236 | BAO84442.1 | 4338 | 10909 | 609 |
| 1087 | I | 3 | CQR46611.1 | 4340 | 10911 | 629 | CQR46610.1 | 4341 | 10912 | 236 | | | | |
| 1088 | I | 4 | BAP87805.1 | 4343 | 10914 | 653 | BAP87806.1 | 4344 | 10915 | 237 | BAP87807.1 | 4345 | 10916 | 212 |
| 1089 | I | 4 | KDP87791.1 | 4347 | 10918 | 663 | KDP87792.1 | 4348 | 10919 | 252 | KDP87793.1 | 4349 | 10920 | 599 |
| 1090 | I | 4 | KQB03823.1 | 4351 | 10922 | 706 | KQB03824.1 | 4352 | 10923 | 244 | KQB03825.1 | 4353 | 10924 | 483 |
| 1091 | I | 4 | KQA99550.1 | 4355 | 10926 | 706 | KQA99549.1 | 4356 | 10927 | 244 | KQA99548.1 | 4357 | 10928 | 483 |
| 1092 | I | 4 | KEY85736.1 | 4359 | 10930 | 730 | KEY85735.1 | 4360 | 10931 | 247 | KEY85734.1 | 4361 | 10932 | 542 |
| 1093 | I | 4 | GAK41823.1 | 4363 | 10934 | 616 | GAK41824.1 | 4364 | 10935 | 258 | GAK41825.1 | 4365 | 10936 | 424 |
| 1094 | I | 4 | KVV58314.1 | 4367 | 10938 | 674 | KVV58315.1 | 4368 | 10939 | 236 | KVV58316.1 | 4369 | 10940 | 475 |
| 1095 | I | 4 | KVX28279.1 | 4371 | 10942 | 674 | KVX28280.1 | 4372 | 10943 | 236 | KVX28281.1 | 4373 | 10944 | 564 |
| 1096 | I | 4 | KVZ97547.1 | 4375 | 10946 | 674 | KVZ97546.1 | 4376 | 10947 | 236 | KVZ97545.1 | 4377 | 10948 | 475 |
| 1097 | I | 4 | AJY51021.1 | 4379 | 10950 | 711 | AJY51020.1 | 4380 | 10951 | 254 | AJY51019.1 | 4381 | 10952 | 530 |
| 1098 | I | 4 | CDX33063.1 | 4383 | 10954 | 723 | CDX33066.1 | 4384 | 10955 | 302 | CDX33069.1 | 4385 | 10956 | 460 |
| 1099 | I | 4 | CDX43778.1 | 4387 | 10958 | 723 | CDX43775.1 | 4388 | 10959 | 302 | CDX43772.1 | 4389 | 10960 | 460 |
| 1100 | I | 3 | KJS80656.1 | 4391 | 10962 | 700 | KJS80657.1 | 4392 | 10963 | 242 | | | | |
| 1101 | I | 4 | KGD90262.1 | 4394 | 10965 | 669 | KGD90263.1 | 4395 | 10966 | 234 | KGD90264.1 | 4396 | 10967 | 570 |
| 1102 | I | 3 | KGJ90758.1 | 4398 | 10969 | 595 | KGJ90759.1 | 4399 | 10970 | 261 | KGJ90760.1 | 4400 | 10971 | 390 |
| 1103 | I | 4 | KOC90088.1 | 4401 | 10972 | 729 | KOC90087.1 | 4402 | 10973 | 246 | KOC90086.1 | 4403 | 10974 | 560 |
| 1104 | I | 4 | KOC94084.1 | 4405 | 10976 | 729 | KOC94083.1 | 4406 | 10977 | 246 | KOC94082.1 | 4407 | 10978 | 560 |
| 1105 | I | 4 | KHF78369.1 | 4409 | 10980 | 687 | KHF78368.1 | 4410 | 10981 | 243 | KHF78367.1 | 4411 | 10982 | 472 |
| 1106 | I | 4 | KQB52476.1 | 4413 | 10984 | 730 | KQB52477.1 | 4414 | 10985 | 247 | KQB52478.1 | 4415 | 10986 | 549 |
| 1107 | I | 3 | | | | | AJP47988.1 | 4417 | 10988 | 236 | AJP47987.1 | 4418 | 10989 | 553 |
| 1108 | I | 4 | KIN89757.1 | 4420 | 10991 | 666 | KIN89556.1 | 4421 | 10992 | 235 | KIN89775.1 | 4422 | 10993 | 595 |
| 1109 | I | 4 | ALQ39732.1 | 4424 | 10995 | 583 | ALQ39733.1 | 4425 | 10996 | 232 | ALQ39734.1 | 4426 | 10997 | 402 |
| 1110 | I | 4 | KIQ83921.1 | 4428 | 10999 | 737 | KIQ83920.1 | 4429 | 11000 | 247 | KIQ83919.1 | 4430 | 11001 | 586 |
| 1111 | I | 4 | KRP95697.1 | 4432 | 11003 | 730 | KRP95696.1 | 4433 | 11004 | 247 | KRP95695.1 | 4434 | 11005 | 542 |
| 1112 | I | 3 | BAQ62924.1 | 4436 | 11007 | 772 | BAQ62925.1 | 4437 | 11008 | 222 | BAQ62926.1 | 4438 | 11009 | 503 |
| 1113 | I | 3 | KJS80968.1 | 4439 | 11010 | 698 | KJS80967.1 | 4440 | 11011 | 254 | KJS80966.1 | 4441 | 11012 | 752 |
| 1114 | I | 4 | KJM39259.1 | 4442 | 11013 | 729 | KJM39258.1 | 4443 | 11014 | 245 | KJM39257.1 | 4444 | 11015 | 504 |
| 1115 | I | 4 | KJJ99150.1 | 4446 | 11017 | 685 | KJJ99540.1 | 4447 | 11018 | 235 | KJJ99149.1 | 4448 | 11019 | 558 |
| 1116 | I | 4 | KJQ91383.1 | 4450 | 11021 | 706 | KJQ91384.1 | 4451 | 11022 | 244 | KJQ91385.1 | 4452 | 11023 | 483 |
| 1117 | I | 4 | KJQ87529.1 | 4454 | 11025 | 706 | KJQ87530.1 | 4455 | 11026 | 244 | KJQ87531.1 | 4456 | 11027 | 483 |
| 1118 | I | 4 | AKI01763.1 | 4458 | 11029 | 720 | AKI01764.1 | 4459 | 11030 | 302 | AKI01765.1 | 4460 | 11031 | 473 |
| 1119 | I | 3 | KMP10823.1 | 4462 | 11033 | 578 | | | | | KMP10824.1 | 4463 | 11034 | 471 |
| 1120 | I | 4 | KJS25182.1 | 4465 | 11036 | 781 | KJS25181.1 | 4466 | 11037 | 254 | KJS25180.1 | 4467 | 11038 | 754 |
| 1121 | I | 3 | KJS49376.1 | 4469 | 11040 | 700 | KJS49375.1 | 4470 | 11041 | 242 | | | | |
| 1122 | I | 4 | KLG11560.1 | 4472 | 11043 | 851 | KLG11561.1 | 4473 | 11044 | 245 | KLG11562.1 | 4474 | 11045 | 597 |
| 1123 | I | 4 | KLP25065.1 | 4476 | 11047 | 729 | KLP25066.1 | 4477 | 11048 | 245 | KLP25067.1 | 4478 | 11049 | 560 |
| 1124 | I | 4 | KLG16385.1 | 4480 | 11051 | 729 | KLG16384.1 | 4481 | 11052 | 245 | KLG16383.1 | 4482 | 11053 | 504 |
| 1125 | I | 4 | AKQ40863.1 | 4484 | 11055 | 641 | AKQ40862.1 | 4485 | 11056 | 242 | AKQ43011.1 | 4486 | 11057 | 440 |
| 1126 | I | 4 | KLR33911.1 | 4488 | 11059 | 729 | KLR33910.1 | 4489 | 11060 | 245 | KLR33909.1 | 4490 | 11061 | 560 |
| 1127 | I | 4 | KLQ90823.1 | 4492 | 11063 | 729 | KLQ90824.1 | 4493 | 11064 | 245 | KLQ90825.1 | 4494 | 11065 | 560 |
| 1128 | I | 4 | KYO07696.1 | 4496 | 11067 | 851 | KYO07695.1 | 4497 | 11068 | 245 | KYO07694.1 | 4498 | 11069 | 597 |
| 1129 | I | 3 | | | | | KYO16913.1 | 4500 | 11071 | 245 | KYO16912.1 | 4501 | 11072 | 560 |
| 1130 | I | 4 | KLP47189.1 | 4503 | 11074 | 729 | KLP47188.1 | 4504 | 11075 | 245 | KLP47187.1 | 4505 | 11076 | 560 |
| 1131 | I | 4 | ALE53304.1 | 4507 | 11078 | 674 | ALE53303.1 | 4508 | 11079 | 236 | ALE53302.1 | 4509 | 11080 | 564 |
| 1132 | I | 4 | KQB93617.1 | 4511 | 11082 | 648 | KQB93618.1 | 4512 | 11083 | 223 | KQB93619.1 | 4513 | 11084 | 426 |
| 1133 | I | 4 | AMN49377.1 | 4515 | 11086 | 688 | AMN49378.1 | 4516 | 11087 | 243 | AMN49379.1 | 4517 | 11088 | 473 |
| 1134 | I | 3 | ALA18584.1 | 4519 | 11090 | 715 | ALA18585.1 | 4520 | 11091 | 306 | ALA18586.1 | 4521 | 11092 | 432 |
| 1135 | I | 4 | ALK96263.1 | 4522 | 11093 | 667 | ALK96264.1 | 4523 | 11094 | 234 | ALK96265.1 | 4524 | 11095 | 578 |
| 1136 | I | 4 | CUJ89804.1 | 4526 | 11097 | 486 | CUJ89796.1 | 4527 | 11098 | 358 | CUJ89790.1 | 4528 | 11099 | 436 |
| 1137 | I | 4 | CUJ83670.1 | 4530 | 11101 | 686 | CUJ83678.1 | 4531 | 11102 | 371 | CUJ83691.1 | 4532 | 11103 | 447 |
| 1138 | I | 4 | CUQ65734.1 | 4534 | 11105 | 605 | CUQ65733.1 | 4535 | 11106 | 244 | CUQ65732.1 | 4536 | 11107 | 479 |
| 1139 | I | 4 | KVV05820.1 | 4538 | 11109 | 730 | KVV05821.1 | 4539 | 11110 | 247 | KVV05822.1 | 4540 | 11111 | 551 |
| 1140 | I | 4 | KVV04346.1 | 4542 | 11113 | 730 | KVV04345.1 | 4543 | 11114 | 247 | KVV04344.1 | 4544 | 11115 | 551 |
| 1141 | I | 4 | KUO77795.1 | 4546 | 11117 | 697 | KUO77796.1 | 4547 | 11118 | 242 | KUO77797.1 | 4548 | 11119 | 399 |
| 1142 | I | 4 | KQM30744.1 | 4550 | 11121 | 483 | KQM30743.1 | 4551 | 11122 | 310 | KQM30742.1 | 4552 | 11123 | 443 |
| 1143 | I | 4 | KQM97531.1 | 4554 | 11125 | 747 | KQM97532.1 | 4555 | 11126 | 227 | KQM97534.1 | 4556 | 11127 | 409 |
| 1144 | I | 4 | KQN80963.1 | 4558 | 11129 | 483 | KQN80962.1 | 4559 | 11130 | 310 | KQN80961.1 | 4560 | 11131 | 443 |
| 1145 | I | 4 | KQQ29436.1 | 4562 | 11133 | 679 | KQQ29437.1 | 4563 | 11134 | 296 | KQQ29438.1 | 4564 | 11135 | 438 |
| 1146 | I | 4 | KQR49970.1 | 4566 | 11137 | 685 | KQR49971.1 | 4567 | 11138 | 236 | KQR49972.1 | 4568 | 11139 | 445 |
| 1147 | I | 3 | KQQ86297.1 | 4570 | 11141 | 679 | KQQ86070.1 | 4571 | 11142 | 332 | KQQ86071.1 | 4572 | 11143 | 437 |
| 1148 | I | 4 | KQS63846.1 | 4573 | 11144 | 677 | KQS63847.1 | 4574 | 11145 | 326 | KQS63848.1 | 4575 | 11146 | 437 |
| 1149 | I | 4 | KQV35757.1 | 4577 | 11148 | 677 | KQV35758.1 | 4578 | 11149 | 326 | KQV35917.1 | 4579 | 11150 | 437 |
| 1150 | I | 4 | KQY95048.1 | 4581 | 11152 | 650 | KQY95047.1 | 4582 | 11153 | 249 | KQY95046.1 | 4583 | 11154 | 354 |
| 1151 | I | 4 | KRD25370.1 | 4585 | 11156 | 725 | KRD25371.1 | 4586 | 11157 | 255 | KRD25372.1 | 4587 | 11158 | 645 |
| 1152 | I | 4 | KRD25864.1 | 4589 | 11160 | 677 | KRD25865.1 | 4590 | 11161 | 326 | KRD26022.1 | 4591 | 11162 | 437 |
| 1153 | I | 4 | KQV20173.1 | 4593 | 11164 | 730 | KQV20174.1 | 4594 | 11165 | 247 | KQV20175.1 | 4595 | 11166 | 542 |
| 1154 | I | 4 | KQX34369.1 | 4597 | 11168 | 654 | KQX34248.1 | 4598 | 11169 | 311 | KQX34249.1 | 4599 | 11170 | 434 |
| 1155 | I | 4 | KRE22550.1 | 4601 | 11172 | 651 | KRE22551.1 | 4602 | 11173 | 306 | KRE22552.1 | 4603 | 11174 | 455 |
| 1156 | I | 4 | KRA26262.1 | 4605 | 11176 | 730 | KRA26261.1 | 4606 | 11177 | 247 | KRA26260.1 | 4607 | 11178 | 542 |
| 1157 | I | 4 | KRA28534.1 | 4609 | 11180 | 650 | KRA28533.1 | 4610 | 11181 | 249 | KRA28532.1 | 4611 | 11182 | 461 |
| 1158 | I | 4 | KRB49806.1 | 4613 | 11184 | 699 | KRB49805.1 | 4614 | 11185 | 314 | KRB49804.1 | 4615 | 11186 | 470 |
| 1159 | I | 4 | KQC46104.1 | 4617 | 11188 | 648 | KQC46105.1 | 4618 | 11189 | 223 | KQC46106.1 | 4619 | 11190 | 426 |
| 1160 | I | 4 | KWV49702.1 | 4621 | 11192 | 676 | KWV49701.1 | 4622 | 11193 | 332 | KWV49700.1 | 4623 | 11194 | 435 |
| 1161 | I | 4 | KUI99562.1 | 4625 | 11196 | 548 | KUI99563.1 | 4626 | 11197 | 244 | KUI99564.1 | 4627 | 11198 | 484 |
| 1162 | I | 4 | KTG21218.1 | 4629 | 11200 | 723 | KTG21219.1 | 4630 | 11201 | 252 | KTG21220.1 | 4631 | 11202 | 564 |
| 1163 | I | 4 | CVJ90686.1 | 4633 | 11204 | 903 | CVJ90696.1 | 4634 | 11205 | 236 | CVJ90705.1 | 4635 | 11206 | 594 |
| 1164 | I | 4 | CVK24348.1 | 4637 | 11208 | 780 | CVK24349.1 | 4638 | 11209 | 241 | CVK24350.1 | 4639 | 11210 | 630 |
| 1165 | I | 4 | CVK22610.1 | 4641 | 11212 | 724 | CVK22609.1 | 4642 | 11213 | 252 | CVK22608.1 | 4643 | 11214 | 546 |
| 1166 | I | 4 | CVK27724.1 | 4645 | 11216 | 785 | CVK27723.1 | 4646 | 11217 | 252 | CVK27722.1 | 4647 | 11218 | 556 |
| 1167 | I | 4 | CVJ14788.1 | 4649 | 11220 | 767 | CVJ14762.1 | 4650 | 11221 | 241 | CVJ14745.1 | 4651 | 11222 | 746 |
| 1168 | I | 4 | CVJ96983.1 | 4653 | 11224 | 844 | CVJ96975.1 | 4654 | 11225 | 240 | CVJ96968.1 | 4655 | 11226 | 658 |
| 1169 | I | 4 | KXS40897.1 | 4657 | 11228 | 657 | KXS40896.1 | 4658 | 11229 | 245 | KXS40895.1 | 4659 | 11230 | 391 |
| 1170 | I | 4 | KXW57569.1 | 4661 | 11232 | 646 | KXW57568.1 | 4662 | 11233 | 237 | KXW57567.1 | 4663 | 11234 | 499 |
| 1171 | I | 4 | EJO46330.1 | 4665 | 11236 | 711 | EJO46331.1 | 4666 | 11237 | 246 | EJO46332.1 | 4667 | 11238 | 560 |
| 1172 | I | 4 | EPY95209.1 | 4669 | 11240 | 851 | EPY95208.1 | 4670 | 11241 | 245 | EPY95207.1 | 4671 | 11242 | 597 |
| 1173 | I | 4 | EUL64248.1 | 4673 | 11244 | 711 | EUL64247.1 | 4674 | 11245 | 246 | EUL64246.1 | 4675 | 11246 | 560 |
| 1174 | I | 4 | AMQ20312.1 | 4677 | 11248 | 648 | AMQ20313.1 | 4678 | 11249 | 223 | AMQ20314.1 | 4679 | 11250 | 426 |
| 1175 | II | 3 | ADB42634.1 | 4681 | 11252 | 472 | ADB42633.1 | 4682 | 11253 | 222 | | | | |
| 1176 | II | 3 | EIA72761.1 | 4684 | 11255 | 432 | EIA72760.1 | 4685 | 11256 | 235 | | | | |
| 1177 | II | 3 | EIA87029.1 | 4687 | 11258 | 432 | EIA87028.1 | 4688 | 11259 | 235 | | | | |
| 1178 | II | 3 | EIA94951.1 | 4690 | 11261 | 432 | EIA94950.1 | 4691 | 11262 | 235 | | | | |
| 1179 | II | 3 | EIA95449.1 | 4693 | 11264 | 432 | EIA95448.1 | 4694 | 11265 | 235 | | | | |
| 1180 | II | 3 | KQH60908.1 | 4696 | 11267 | 432 | KQH60907.1 | 4697 | 11268 | 235 | | | | |
| 1181 | II | 3 | KQI25488.1 | 4699 | 11270 | 432 | KQI25489.1 | 4700 | 11271 | 235 | | | | |
| 1182 | II | 3 | KQI25380.1 | 4702 | 11273 | 432 | KQI25381.1 | 4703 | 11274 | 235 | | | | |
| 1183 | II | 3 | KRS58891.1 | 4705 | 11276 | 432 | KRS58892.1 | 4706 | 11277 | 235 | | | | |
| 1184 | II | 3 | KRS89289.1 | 4708 | 11279 | 432 | KRS89290.1 | 4709 | 11280 | 234 | | | | |
| 1185 | II | 3 | KEA44658.1 | 4711 | 11282 | 480 | KEA44659.1 | 4712 | 11283 | 239 | | | | |
| 1186 | II | 3 | ALX13353.1 | 4714 | 11285 | 427 | ALX11999.1 | 4715 | 11286 | 230 | | | | |
| 1187 | II | 3 | AMB80664.1 | 4717 | 11288 | 547 | AMB81837.1 | 4718 | 11289 | 240 | | | | |
| 1188 | II | 3 | CZG58504.1 | 4720 | 11291 | 498 | CZG58452.1 | 4721 | 11292 | 230 | | | | |
| 1189 | II | 3 | CZH63906.1 | 4723 | 11294 | 498 | CZH63878.1 | 4724 | 11295 | 230 | | | | |
| 1190 | II | 3 | CZG92946.1 | 4726 | 11297 | 498 | CZG92895.1 | 4727 | 11298 | 230 | | | | |
| 1191 | II | 3 | CZI24914.1 | 4729 | 11300 | 498 | CZI24954.1 | 4730 | 11301 | 230 | | | | |
| 1192 | II | 3 | CZI29146.1 | 4732 | 11303 | 498 | CZI29107.1 | 4733 | 11304 | 230 | | | | |
| 1193 | II | 3 | CZH75364.1 | 4735 | 11306 | 498 | CZH75396.1 | 4736 | 11307 | 230 | | | | |
| 1194 | II | 3 | CZI28533.1 | 4738 | 11309 | 498 | CZI28487.1 | 4739 | 11310 | 230 | | | | |
| 1195 | II | 3 | CZQ90586.1 | 4741 | 11312 | 498 | CZQ90595.1 | 4742 | 11313 | 230 | | | | |
| 1196 | II | 3 | EEZ95918.1 | 4744 | 11315 | 455 | EEZ95917.1 | 4745 | 11316 | 230 | | | | |
| 1197 | II | 3 | ADG61932.1 | 4747 | 11318 | 465 | ADG61931.1 | 4748 | 11319 | 228 | | | | |
| 1198 | II | 3 | EGE12537.1 | 4750 | 11321 | 465 | EGE12536.1 | 4751 | 11322 | 204 | | | | |
| 1199 | II | 3 | EGE10945.1 | 4753 | 11324 | 465 | EGE10944.1 | 4754 | 11325 | 204 | | | | |
| 1200 | II | 3 | EGE11787.1 | 4756 | 11327 | 465 | EGE11786.1 | 4757 | 11328 | 204 | | | | |
| 1201 | II | 3 | EGE15771.1 | 4759 | 11330 | 342 | EGE15772.1 | 4760 | 11331 | 204 | | | | |
| 1202 | II | 3 | EGE17285.1 | 4762 | 11333 | 465 | EGE17284.1 | 4763 | 11334 | 204 | | | | |
| 1203 | II | 3 | EGE18525.1 | 4765 | 11336 | 465 | EGE18524.1 | 4766 | 11337 | 204 | | | | |
| 1204 | II | 3 | EGE26180.1 | 4768 | 11339 | 378 | EGE26179.1 | 4769 | 11340 | 204 | | | | |
| 1205 | II | 3 | EGE24867.1 | 4771 | 11342 | 465 | EGE24866.1 | 4772 | 11343 | 204 | | | | |
| 1206 | II | 3 | EKF83143.1 | 4774 | 11345 | 465 | EKF83142.1 | 4775 | 11346 | 214 | | | | |
| 1207 | II | 3 | AIT43974.1 | 4777 | 11348 | 378 | AIT43973.1 | 4778 | 11349 | 238 | | | | |
| 1208 | II | 3 | ALD78152.1 | 4780 | 11351 | 498 | ALD78151.1 | 4781 | 11352 | 235 | | | | |
| 1209 | II | 3 | KJI56238.1 | 4783 | 11354 | 550 | KJI56239.1 | 4784 | 11355 | 235 | | | | |
| 1210 | II | 3 | KKW50395.1 | 4786 | 11357 | 180 | KKW50401.1 | 4787 | 11358 | 226 | | | | |
| 1211 | II | 3 | KFF70136.1 | 4789 | 11360 | 459 | KFF70137.1 | 4790 | 11361 | 244 | | | | |
| 1212 | II | 3 | KHS98349.1 | 4792 | 11363 | 459 | KHS98348.1 | 4793 | 11364 | 244 | | | | |
| 1213 | II | 3 | AAG59482.1 | 4795 | 11366 | 550 | AAG59481.1 | 4796 | 11367 | 235 | | | | |
| 1214 | II | 3 | BAB38681.1 | 4798 | 11369 | 550 | BAB38680.1 | 4799 | 11370 | 235 | | | | |
| 1215 | II | 3 | ACA79490.1 | 4801 | 11372 | 550 | ACA79491.1 | 4802 | 11373 | 235 | | | | |
| 1216 | II | 3 | ACB17340.1 | 4804 | 11375 | 550 | ACB16649.1 | 4805 | 11376 | 235 | | | | |
| 1217 | II | 3 | ACI35160.1 | 4807 | 11378 | 550 | ACI35732.1 | 4808 | 11379 | 235 | | | | |
| 1218 | II | 3 | ACT75062.1 | 4810 | 11381 | 550 | ACT75061.1 | 4811 | 11382 | 235 | | | | |
| 1219 | II | 3 | ADD59517.1 | 4813 | 11384 | 550 | ADD59516.1 | 4814 | 11385 | 235 | | | | |
| 1220 | II | 3 | EEC30753.1 | 4816 | 11387 | 550 | EEC30190.1 | 4817 | 11388 | 235 | | | | |
| 1221 | II | 3 | EFK27048.1 | 4819 | 11390 | 550 | EFK27047.1 | 4820 | 11391 | 235 | | | | |
| 1222 | II | 3 | EDU34174.1 | 4822 | 11393 | 550 | EDU34140.1 | 4823 | 11394 | 235 | | | | |
| 1223 | II | 3 | EDU54291.1 | 4825 | 11396 | 550 | EDU54290.1 | 4826 | 11397 | 235 | | | | |
| 1224 | II | 3 | EDU68759.1 | 4828 | 11399 | 550 | EDU68786.1 | 4829 | 11400 | 235 | | | | |
| 1225 | II | 3 | EDU76183.1 | 4831 | 11402 | 550 | EDU76267.1 | 4832 | 11403 | 235 | | | | |
| 1226 | II | 3 | EDU82627.1 | 4834 | 11405 | 550 | EDU82726.1 | 4835 | 11406 | 235 | | | | |
| 1227 | II | 3 | EDU87443.1 | 4837 | 11408 | 550 | EDU87344.1 | 4838 | 11409 | 235 | | | | |
| 1228 | II | 3 | EDU93482.1 | 4840 | 11411 | 550 | EDU93378.1 | 4841 | 11412 | 235 | | | | |
| 1229 | II | 3 | EDU97879.1 | 4843 | 11414 | 550 | EDU97966.1 | 4844 | 11415 | 235 | | | | |
| 1230 | II | 3 | EGI28993.1 | 4846 | 11417 | 550 | EGI28991.1 | 4847 | 11418 | 159 | | | | |
| 1231 | II | 3 | EDZ75683.1 | 4849 | 11420 | 550 | EDZ75201.1 | 4850 | 11421 | 235 | | | | |
| 1232 | II | 3 | EDZ82225.1 | 4852 | 11423 | 550 | EDZ82316.1 | 4853 | 11424 | 235 | | | | |
| 1233 | II | 3 | EDZ89786.1 | 4855 | 11426 | 550 | EDZ89331.1 | 4856 | 11427 | 235 | | | | |
| 1234 | II | 3 | EFX08673.1 | 4858 | 11429 | 550 | EFX08672.1 | 4859 | 11430 | 235 | | | | |
| 1235 | II | 3 | EFX13462.1 | 4861 | 11432 | 550 | EFX13461.1 | 4862 | 11433 | 235 | | | | |
| 1236 | II | 3 | EFX18239.1 | 4864 | 11435 | 550 | EFX18238.1 | 4865 | 11436 | 235 | | | | |
| 1237 | II | 3 | EFX28078.1 | 4867 | 11438 | 498 | EFX28077.1 | 4868 | 11439 | 235 | | | | |
| 1238 | II | 3 | EFX32591.1 | 4870 | 11441 | 498 | EFX32590.1 | 4871 | 11442 | 235 | | | | |
| 1239 | II | 3 | EGD70493.1 | 4873 | 11444 | 550 | EGD70492.1 | 4874 | 11445 | 206 | | | | |
| 1240 | II | 3 | EGD69129.1 | 4876 | 11447 | 550 | EGD69130.1 | 4877 | 11448 | 206 | | | | |
| 1241 | II | 3 | EFW65234.1 | 4879 | 11450 | 550 | EFW65235.1 | 4880 | 11451 | 206 | | | | |
| 1242 | II | 3 | AEZ43447.1 | 4882 | 11453 | 550 | AEZ43446.1 | 4883 | 11454 | 235 | | | | |
| 1243 | II | 3 | EHU86059.1 | 4885 | 11456 | 550 | EHU86058.1 | 4886 | 11457 | 210 | | | | |
| 1244 | II | 3 | EHU89504.1 | 4888 | 11459 | 498 | EHU89503.1 | 4889 | 11460 | 76 | | | | |
| 1245 | II | 3 | EHV18499.1 | 4891 | 11462 | 498 | EHV18498.1 | 4892 | 11463 | 210 | | | | |
| 1246 | II | 3 | EHV31734.1 | 4894 | 11465 | 550 | EHV31797.1 | 4895 | 11466 | 206 | | | | |
| 1247 | II | 3 | EHV46593.1 | 4897 | 11468 | 498 | EHV46592.1 | 4898 | 11469 | 210 | | | | |
| 1248 | II | 3 | EIF87146.1 | 4900 | 11471 | 550 | EIF87145.1 | 4901 | 11472 | 235 | | | | |
| 1249 | II | 3 | AFJ31993.1 | 4903 | 11474 | 550 | AFJ31992.1 | 4904 | 11475 | 235 | | | | |
| 1250 | II | 3 | EIN15909.1 | 4906 | 11477 | 550 | EIN15908.1 | 4907 | 11478 | 210 | | | | |
| 1251 | II | 3 | EIP63310.1 | 4909 | 11480 | 550 | EIP63578.1 | 4910 | 11481 | 206 | | | | |
| 1252 | II | 3 | EIP71583.1 | 4912 | 11483 | 550 | EIP71554.1 | 4913 | 11484 | 202 | | | | |
| 1253 | II | 3 | EIO79100.1 | 4915 | 11486 | 550 | EIO79099.1 | 4916 | 11487 | 210 | | | | |
| 1254 | II | 3 | EIN94365.1 | 4918 | 11489 | 550 | EIN94459.1 | 4919 | 11490 | 202 | | | | |
| 1255 | II | 3 | EIO32395.1 | 4921 | 11492 | 550 | EIO32394.1 | 4922 | 11493 | 210 | | | | |
| 1256 | II | 3 | EIO45819.1 | 4924 | 11495 | 550 | EIO46021.1 | 4925 | 11496 | 210 | | | | |
| 1257 | II | 3 | EIO63273.1 | 4927 | 11498 | 550 | EIO63762.1 | 4928 | 11499 | 210 | | | | |
| 1258 | II | 3 | EIO69657.1 | 4930 | 11501 | 550 | EIO69656.1 | 4931 | 11502 | 210 | | | | |
| 1259 | II | 3 | EIO86334.1 | 4933 | 11504 | 550 | EIO86307.1 | 4934 | 11505 | 206 | | | | |
| 1260 | II | 3 | EIP72925.1 | 4936 | 11507 | 550 | EIP72924.1 | 4937 | 11508 | 210 | | | | |
| 1261 | II | 3 | EIP72313.1 | 4939 | 11510 | 550 | EIP72312.1 | 4940 | 11511 | 210 | | | | |
| 1262 | II | 3 | EIP50029.1 | 4942 | 11513 | 550 | EIP50028.1 | 4943 | 11514 | 206 | | | | |
| 1263 | II | 3 | EIP48998.1 | 4945 | 11516 | 550 | EIP49007.1 | 4946 | 11517 | 205 | | | | |
| 1264 | II | 3 | EIP40275.1 | 4948 | 11519 | 550 | EIP40258.1 | 4949 | 11520 | 235 | | | | |
| 1265 | II | 3 | EIP35349.1 | 4951 | 11522 | 550 | EIP35359.1 | 4952 | 11523 | 210 | | | | |
| 1266 | II | 3 | EIP27300.1 | 4954 | 11525 | 452 | EIP27306.1 | 4955 | 11526 | 210 | | | | |
| 1267 | II | 3 | EIP38853.1 | 4957 | 11528 | 550 | EIP38852.1 | 4958 | 11529 | 210 | | | | |
| 1268 | II | 3 | EIP33942.1 | 4960 | 11531 | 550 | EIP33941.1 | 4961 | 11532 | 210 | | | | |
| 1269 | II | 3 | EIP10352.1 | 4963 | 11534 | 550 | EIP10349.1 | 4964 | 11535 | 210 | | | | |
| 1270 | II | 3 | EIP05667.1 | 4966 | 11537 | 550 | EIP05660.1 | 4967 | 11538 | 210 | | | | |
| 1271 | II | 3 | EIP03403.1 | 4969 | 11540 | 452 | EIP03404.1 | 4970 | 11541 | 210 | | | | |
| 1272 | II | 3 | EIO92501.1 | 4972 | 11543 | 550 | EIO92517.1 | 4973 | 11544 | 210 | | | | |
| 1273 | II | 3 | EIO87483.1 | 4975 | 11546 | 550 | EIO87493.1 | 4976 | 11547 | 202 | | | | |
| 1274 | II | 3 | EIN16733.1 | 4978 | 11549 | 550 | EIN16732.1 | 4979 | 11550 | 210 | | | | |
| 1275 | II | 3 | EIN15657.1 | 4981 | 11552 | 550 | EIN15629.1 | 4982 | 11553 | 210 | | | | |
| 1276 | II | 3 | EIN33040.1 | 4984 | 11555 | 550 | EIN33050.1 | 4985 | 11556 | 210 | | | | |
| 1277 | II | 3 | EIO86817.1 | 4987 | 11558 | 191 | EIO86816.1 | 4988 | 11559 | 202 | | | | |
| 1278 | II | 3 | EIO65815.1 | 4990 | 11561 | 550 | EIO65813.1 | 4991 | 11562 | 210 | | | | |
| 1279 | II | 3 | EIO79447.1 | 4993 | 11564 | 550 | EIO79464.1 | 4994 | 11565 | 210 | | | | |
| 1280 | II | 3 | EIO49130.1 | 4996 | 11567 | 550 | EIO49202.1 | 4997 | 11568 | 202 | | | | |
| 1281 | II | 3 | EIO29777.1 | 4999 | 11570 | 550 | EIO29776.1 | 5000 | 11571 | 206 | | | | |
| 1282 | II | 3 | EIO24705.1 | 5002 | 11573 | 452 | EIO24703.1 | 5003 | 11574 | 210 | | | | |
| 1283 | II | 3 | EIO11193.1 | 5005 | 11576 | 550 | EIO11192.1 | 5006 | 11577 | 210 | | | | |
| 1284 | II | 3 | EIO07409.1 | 5008 | 11579 | 550 | EIO07417.1 | 5009 | 11580 | 210 | | | | |
| 1285 | II | 3 | EIO07023.1 | 5011 | 11582 | 550 | EIO07031.1 | 5012 | 11583 | 210 | | | | |
| 1286 | II | 3 | EIO09663.1 | 5014 | 11585 | 550 | EIO09672.1 | 5015 | 11586 | 206 | | | | |
| 1287 | II | 3 | EIN89796.1 | 5017 | 11588 | 550 | EIN89896.1 | 5018 | 11589 | 210 | | | | |
| 1288 | II | 3 | EIN88185.1 | 5020 | 11591 | 550 | EIN88192.1 | 5021 | 11592 | 210 | | | | |
| 1289 | II | 3 | EIN85439.1 | 5023 | 11594 | 550 | EIN85471.1 | 5024 | 11595 | 210 | | | | |
| 1290 | II | 3 | EIN69802.1 | 5026 | 11597 | 550 | EIN69801.1 | 5027 | 11598 | 210 | | | | |
| 1291 | II | 3 | EIN65413.1 | 5029 | 11600 | 550 | EIN65412.1 | 5030 | 11601 | 202 | | | | |
| 1292 | II | 3 | EIN68646.1 | 5032 | 11603 | 550 | EIN68645.1 | 5033 | 11604 | 210 | | | | |
| 1293 | II | 3 | EIN52366.1 | 5035 | 11606 | 550 | EIN52365.1 | 5036 | 11607 | 210 | | | | |
| 1294 | II | 3 | EIN49112.1 | 5038 | 11609 | 550 | EIN49111.1 | 5039 | 11610 | 210 | | | | |
| 1295 | II | 3 | EIN33836.1 | 5041 | 11612 | 550 | EIN33835.1 | 5042 | 11613 | 210 | | | | |
| 1296 | II | 3 | EIN35239.1 | 5044 | 11615 | 550 | EIN35238.1 | 5045 | 11616 | 202 | | | | |
| 1297 | II | 3 | EKG95781.1 | 5047 | 11618 | 550 | EKG95780.1 | 5048 | 11619 | 205 | | | | |
| 1298 | II | 3 | EKH07680.1 | 5050 | 11621 | 550 | EKH07679.1 | 5051 | 11622 | 206 | | | | |
| 1299 | II | 3 | EKJ54995.1 | 5053 | 11624 | 550 | EKJ54994.1 | 5054 | 11625 | 210 | | | | |
| 1300 | II | 3 | EKJ48134.1 | 5056 | 11627 | 550 | EKJ48188.1 | 5057 | 11628 | 210 | | | | |
| 1301 | II | 3 | EKJ52904.1 | 5059 | 11630 | 550 | EKJ52873.1 | 5060 | 11631 | 210 | | | | |
| 1302 | II | 3 | EKJ36498.1 | 5062 | 11633 | 550 | EKJ36497.1 | 5063 | 11634 | 202 | | | | |
| 1303 | II | 3 | EKJ47662.1 | 5065 | 11636 | 550 | EKJ47661.1 | 5066 | 11637 | 210 | | | | |
| 1304 | II | 3 | EKJ36051.1 | 5068 | 11639 | 550 | EKJ36042.1 | 5069 | 11640 | 210 | | | | |
| 1305 | II | 3 | EKJ20862.1 | 5071 | 11642 | 498 | EKJ20861.1 | 5072 | 11643 | 210 | | | | |
| 1306 | II | 3 | EKJ19824.1 | 5074 | 11645 | 550 | EKJ19823.1 | 5075 | 11646 | 206 | | | | |
| 1307 | II | 3 | EKJ00851.1 | 5077 | 11648 | 550 | EKJ00850.1 | 5078 | 11649 | 210 | | | | |
| 1308 | II | 3 | EKI92952.1 | 5080 | 11651 | 550 | EKI92951.1 | 5081 | 11652 | 210 | | | | |
| 1309 | II | 3 | EKJ05352.1 | 5083 | 11654 | 550 | EKJ05390.1 | 5084 | 11655 | 202 | | | | |
| 1310 | II | 3 | EKI82675.1 | 5086 | 11657 | 550 | EKI82674.1 | 5087 | 11658 | 210 | | | | |
| 1311 | II | 3 | EKI91870.1 | 5089 | 11660 | 550 | EKI91869.1 | 5090 | 11661 | 210 | | | | |
| 1312 | II | 3 | EKI72763.1 | 5092 | 11663 | 550 | EKI72762.1 | 5093 | 11664 | 210 | | | | |
| 1313 | II | 3 | EKI64838.1 | 5095 | 11666 | 550 | EKI64837.1 | 5096 | 11667 | 210 | | | | |
| 1314 | II | 3 | EKI59123.1 | 5098 | 11669 | 550 | EKI59122.1 | 5099 | 11670 | 210 | | | | |
| 1315 | II | 3 | EKI57117.1 | 5101 | 11672 | 550 | EKI57139.1 | 5102 | 11673 | 206 | | | | |
| 1316 | II | 3 | EKI46667.1 | 5104 | 11675 | 550 | EKI46666.1 | 5105 | 11676 | 210 | | | | |
| 1317 | II | 3 | EKI33386.1 | 5107 | 11678 | 498 | EKI33385.1 | 5108 | 11679 | 210 | | | | |
| 1318 | II | 3 | EKH20917.1 | 5110 | 11681 | 550 | EKH20916.1 | 5111 | 11682 | 210 | | | | |
| 1319 | II | 3 | EKH25286.1 | 5113 | 11684 | 550 | EKH25285.1 | 5114 | 11685 | 210 | | | | |
| 1320 | II | 3 | EKH13352.1 | 5116 | 11687 | 550 | EKH13351.1 | 5117 | 11688 | 210 | | | | |
| 1321 | II | 3 | EKH36315.1 | 5119 | 11690 | 550 | EKH36314.1 | 5120 | 11691 | 210 | | | | |
| 1322 | II | 3 | EKH26863.1 | 5122 | 11693 | 550 | EKH26862.1 | 5123 | 11694 | 210 | | | | |
| 1323 | II | 3 | EKH48829.1 | 5125 | 11696 | 550 | EKH48828.1 | 5126 | 11697 | 210 | | | | |
| 1324 | II | 3 | EKH53678.1 | 5128 | 11699 | 550 | EKH53677.1 | 5129 | 11700 | 210 | | | | |
| 1325 | II | 3 | EKH61580.1 | 5131 | 11702 | 550 | EKH61520.1 | 5132 | 11703 | 210 | | | | |
| 1326 | II | 3 | EKH63165.1 | 5134 | 11705 | 550 | EKH63164.1 | 5135 | 11706 | 210 | | | | |
| 1327 | II | 3 | EKH71847.1 | 5137 | 11708 | 550 | EKH71789.1 | 5138 | 11709 | 210 | | | | |
| 1328 | II | 3 | EKH63848.1 | 5140 | 11711 | 550 | EKH63847.1 | 5141 | 11712 | 210 | | | | |
| 1329 | II | 3 | EKH83439.1 | 5143 | 11714 | 550 | EKH83457.1 | 5144 | 11715 | 210 | | | | |
| 1330 | II | 3 | EKH87900.1 | 5146 | 11717 | 550 | EKH87899.1 | 5147 | 11718 | 210 | | | | |
| 1331 | II | 3 | EKH95565.1 | 5149 | 11720 | 550 | EKH95564.1 | 5150 | 11721 | 210 | | | | |
| 1332 | II | 3 | EKI01262.1 | 5152 | 11723 | 550 | EKI01261.1 | 5153 | 11724 | 210 | | | | |
| 1333 | II | 3 | EKI02731.1 | 5155 | 11726 | 550 | EKI02730.1 | 5156 | 11727 | 210 | | | | |
| 1334 | II | 3 | EKH96738.1 | 5158 | 11729 | 550 | EKH96737.1 | 5159 | 11730 | 210 | | | | |
| 1335 | II | 3 | EKI03040.1 | 5161 | 11732 | 550 | EKI03039.1 | 5162 | 11733 | 210 | | | | |
| 1336 | II | 3 | EKI06059.1 | 5164 | 11735 | 550 | EKI06058.1 | 5165 | 11736 | 210 | | | | |
| 1337 | II | 3 | EKK37394.1 | 5167 | 11738 | 550 | EKK37393.1 | 5168 | 11739 | 210 | | | | |
| 1338 | II | 3 | EKK71766.1 | 5170 | 11741 | 550 | EKK71765.1 | 5171 | 11742 | 210 | | | | |
| 1339 | II | 3 | EKK91183.1 | 5173 | 11744 | 550 | EKK91182.1 | 5174 | 11745 | 210 | | | | |
| 1340 | II | 3 | EKK36994.1 | 5176 | 11747 | 550 | EKK36843.1 | 5177 | 11748 | 206 | | | | |
| 1341 | II | 3 | EKK37977.1 | 5179 | 11750 | 550 | EKK37976.1 | 5180 | 11751 | 206 | | | | |
| 1342 | II | 3 | EKK61089.1 | 5182 | 11753 | 550 | EKK61088.1 | 5183 | 11754 | 205 | | | | |
| 1343 | II | 3 | EKK52332.1 | 5185 | 11756 | 550 | EKK52331.1 | 5186 | 11757 | 210 | | | | |
| 1344 | II | 3 | EKK67939.1 | 5188 | 11759 | 550 | EKK67938.1 | 5189 | 11760 | 210 | | | | |
| 1345 | II | 3 | EKK62291.1 | 5191 | 11762 | 550 | EKK62290.1 | 5192 | 11763 | 210 | | | | |
| 1346 | II | 3 | EKK87667.1 | 5194 | 11765 | 550 | EKK87666.1 | 5195 | 11766 | 210 | | | | |
| 1347 | II | 3 | EKV88765.1 | 5197 | 11768 | 550 | EKV88764.1 | 5198 | 11769 | 210 | | | | |
| 1348 | II | 3 | EKV85098.1 | 5200 | 11771 | 550 | EKV85097.1 | 5201 | 11772 | 210 | | | | |
| 1349 | II | 3 | EKV70652.1 | 5203 | 11774 | 550 | EKV70651.1 | 5204 | 11775 | 210 | | | | |
| 1350 | II | 3 | EKW05771.1 | 5206 | 11777 | 550 | EKW05770.1 | 5207 | 11778 | 202 | | | | |
| 1351 | II | 3 | EKV91529.1 | 5209 | 11780 | 550 | EKV91591.1 | 5210 | 11781 | 210 | | | | |
| 1352 | II | 3 | EKW03290.1 | 5212 | 11783 | 550 | EKW03180.1 | 5213 | 11784 | 210 | | | | |
| 1353 | II | 3 | EKW04243.1 | 5215 | 11786 | 550 | EKW04288.1 | 5216 | 11787 | 210 | | | | |
| 1354 | II | 3 | EKW19024.1 | 5218 | 11789 | 550 | EKW19023.1 | 5219 | 11790 | 210 | | | | |
| 1355 | II | 3 | EKW08487.1 | 5221 | 11792 | 550 | EKW08486.1 | 5222 | 11793 | 210 | | | | |
| 1356 | II | 3 | EKW71371.1 | 5224 | 11795 | 550 | EKW71370.1 | 5225 | 11796 | 210 | | | | |
| 1357 | II | 3 | EKW96655.1 | 5227 | 11798 | 550 | EKW96654.1 | 5228 | 11799 | 202 | | | | |
| 1358 | II | 3 | EKW96260.1 | 5230 | 11801 | 550 | EKW96259.1 | 5231 | 11802 | 210 | | | | |
| 1359 | II | 3 | EKW76356.1 | 5233 | 11804 | 498 | EKW76355.1 | 5234 | 11805 | 210 | | | | |
| 1360 | II | 3 | EKW75842.1 | 5236 | 11807 | 550 | EKW75841.1 | 5237 | 11808 | 206 | | | | |
| 1361 | II | 3 | EKW68012.1 | 5239 | 11810 | 550 | EKW68011.1 | 5240 | 11811 | 202 | | | | |
| 1362 | II | 3 | EKW74793.1 | 5242 | 11813 | 550 | EKW74753.1 | 5243 | 11814 | 210 | | | | |
| 1363 | II | 3 | EKW43906.1 | 5245 | 11816 | 550 | EKW43905.1 | 5246 | 11817 | 210 | | | | |
| 1364 | II | 3 | EKW70675.1 | 5248 | 11819 | 550 | EKW70674.1 | 5249 | 11820 | 210 | | | | |
| 1365 | II | 3 | EKW38124.1 | 5251 | 11822 | 550 | EKW38123.1 | 5252 | 11823 | 210 | | | | |
| 1366 | II | 3 | EKW21071.1 | 5254 | 11825 | 352 | EKW21070.1 | 5255 | 11826 | 210 | | | | |
| 1367 | II | 3 | EKW36043.1 | 5257 | 11828 | 550 | EKW36042.1 | 5258 | 11829 | 202 | | | | |
| 1368 | II | 3 | EKW36718.1 | 5260 | 11831 | 550 | EKW36717.1 | 5261 | 11832 | 210 | | | | |
| 1369 | II | 3 | EKW51278.1 | 5263 | 11834 | 550 | EKW51302.1 | 5264 | 11835 | 210 | | | | |
| 1370 | II | 3 | EKY34591.1 | 5266 | 11837 | 550 | EKY34590.1 | 5267 | 11838 | 210 | | | | |
| 1371 | II | 3 | EKY34729.1 | 5269 | 11840 | 498 | EKY34728.1 | 5270 | 11841 | 206 | | | | |
| 1372 | II | 3 | ELI49200.1 | 5272 | 11843 | 550 | ELI49199.1 | 5273 | 11844 | 235 | | | | |
| 1373 | II | 3 | ELV23654.1 | 5275 | 11846 | 550 | ELV23653.1 | 5276 | 11847 | 210 | | | | |
| 1374 | II | 3 | ELV30876.1 | 5278 | 11849 | 550 | ELV30875.1 | 5279 | 11850 | 210 | | | | |
| 1375 | II | 3 | ELV62885.1 | 5281 | 11852 | 498 | ELV62893.1 | 5282 | 11853 | 210 | | | | |
| 1376 | II | 3 | ELV61722.1 | 5284 | 11855 | 443 | ELV61721.1 | 5285 | 11856 | 210 | | | | |
| 1377 | II | 3 | ELV63259.1 | 5287 | 11858 | 550 | ELV63258.1 | 5288 | 11859 | 210 | | | | |
| 1378 | II | 3 | ELV91642.1 | 5290 | 11861 | 550 | ELV91641.1 | 5291 | 11862 | 206 | | | | |
| 1379 | II | 3 | ELV93149.1 | 5293 | 11864 | 550 | ELV93148.1 | 5294 | 11865 | 210 | | | | |
| 1380 | II | 3 | ELW21805.1 | 5296 | 11867 | 550 | ELW21804.1 | 5297 | 11868 | 205 | | | | |
| 1381 | II | 3 | ELW24262.1 | 5299 | 11870 | 550 | ELW24261.1 | 5300 | 11871 | 210 | | | | |
| 1382 | II | 3 | ELW27707.1 | 5302 | 11873 | 550 | ELW27706.1 | 5303 | 11874 | 210 | | | | |
| 1383 | II | 3 | ELW23231.1 | 5305 | 11876 | 550 | ELW23230.1 | 5306 | 11877 | 202 | | | | |
| 1384 | II | 3 | ELW28316.1 | 5308 | 11879 | 550 | ELW28315.1 | 5309 | 11880 | 202 | | | | |
| 1385 | II | 3 | ELW46370.1 | 5311 | 11882 | 550 | ELW46369.1 | 5312 | 11883 | 210 | | | | |
| 1386 | II | 3 | ELV14533.1 | 5314 | 11885 | 550 | ELV14532.1 | 5315 | 11886 | 210 | | | | |
| 1387 | II | 3 | ELV31270.1 | 5317 | 11888 | 550 | ELV31244.1 | 5318 | 11889 | 210 | | | | |
| 1388 | II | 3 | ELV35522.1 | 5320 | 11891 | 550 | ELV35521.1 | 5321 | 11892 | 206 | | | | |
| 1389 | II | 3 | ELV44403.1 | 5323 | 11894 | 452 | ELV44402.1 | 5324 | 11895 | 235 | | | | |
| 1390 | II | 3 | ELV47175.1 | 5326 | 11897 | 550 | ELV47167.1 | 5327 | 11898 | 206 | | | | |
| 1391 | II | 3 | ELV76553.1 | 5329 | 11900 | 550 | ELV76552.1 | 5330 | 11901 | 202 | | | | |
| 1392 | II | 3 | ELV98805.1 | 5332 | 11903 | 550 | ELV98804.1 | 5333 | 11904 | 210 | | | | |
| 1393 | II | 3 | ELV75967.1 | 5335 | 11906 | 550 | ELV75966.1 | 5336 | 11907 | 210 | | | | |
| 1394 | II | 3 | ELV90962.1 | 5338 | 11909 | 550 | ELV90961.1 | 5339 | 11910 | 235 | | | | |
| 1395 | II | 3 | ELW12424.1 | 5341 | 11912 | 550 | ELW12423.1 | 5342 | 11913 | 210 | | | | |
| 1396 | II | 3 | ELV15308.1 | 5344 | 11915 | 550 | ELV15307.1 | 5345 | 11916 | 210 | | | | |
| 1397 | II | 3 | EMR91942.1 | 5347 | 11918 | 550 | EMR91943.1 | 5348 | 11919 | 235 | | | | |
| 1398 | II | 3 | EMS03600.1 | 5350 | 11921 | 550 | EMS03599.1 | 5351 | 11922 | 235 | | | | |
| 1399 | II | 3 | ELE41780.1 | 5353 | 11924 | 550 | ELE41779.1 | 5354 | 11925 | 235 | | | | |
| 1400 | II | 3 | ELF28432.1 | 5356 | 11927 | 550 | ELF28431.1 | 5357 | 11928 | 235 | | | | |
| 1401 | II | 3 | ELG08155.1 | 5359 | 11930 | 550 | ELG08154.1 | 5360 | 11931 | 235 | | | | |
| 1402 | II | 3 | ELH63190.1 | 5362 | 11933 | 550 | ELH63189.1 | 5363 | 11934 | 235 | | | | |
| 1403 | II | 3 | ENE27218.1 | 5365 | 11936 | 550 | ENE27216.1 | 5366 | 11937 | 206 | | | | |
| 1404 | II | 3 | EQO57392.1 | 5368 | 11939 | 550 | EQO57391.1 | 5369 | 11940 | 235 | | | | |
| 1405 | II | 3 | EQO66403.1 | 5371 | 11942 | 550 | EQO66402.1 | 5372 | 11943 | 235 | | | | |
| 1406 | II | 3 | EQT31739.1 | 5374 | 11945 | 550 | EQT31738.1 | 5375 | 11946 | 235 | | | | |
| 1407 | II | 3 | EQT84558.1 | 5377 | 11948 | 550 | EQT84557.1 | 5378 | 11949 | 235 | | | | |
| 1408 | II | 3 | ERB08804.1 | 5380 | 11951 | 550 | ERB08803.1 | 5381 | 11952 | 235 | | | | |
| 1409 | II | 3 | ERB13352.1 | 5383 | 11954 | 550 | ERB13353.1 | 5384 | 11955 | 235 | | | | |
| 1410 | II | 3 | ERC00102.1 | 5386 | 11957 | 550 | ERC00101.1 | 5387 | 11958 | 206 | | | | |
| 1411 | II | 3 | ERB68141.1 | 5389 | 11960 | 550 | ERB68140.1 | 5390 | 11961 | 210 | | | | |
| 1412 | II | 3 | ERB88973.1 | 5392 | 11963 | 550 | ERB88972.1 | 5393 | 11964 | 210 | | | | |
| 1413 | II | 3 | ERC15358.1 | 5395 | 11966 | 550 | ERC15203.1 | 5396 | 11967 | 202 | | | | |
| 1414 | II | 3 | ERB79215.1 | 5398 | 11969 | 550 | ERB79214.1 | 5399 | 11970 | 205 | | | | |
| 1415 | II | 3 | ERB91962.1 | 5401 | 11972 | 550 | ERB92027.1 | 5402 | 11973 | 205 | | | | |
| 1416 | II | 3 | ERC08311.1 | 5404 | 11975 | 550 | ERC08310.1 | 5405 | 11976 | 210 | | | | |
| 1417 | II | 3 | ERC32305.1 | 5407 | 11978 | 550 | ERC32304.1 | 5408 | 11979 | 210 | | | | |
| 1418 | II | 3 | ERC25309.1 | 5410 | 11981 | 550 | ERC25308.1 | 5411 | 11982 | 205 | | | | |
| 1419 | II | 3 | ERC09303.1 | 5413 | 11984 | 550 | ERC09302.1 | 5414 | 11985 | 210 | | | | |
| 1420 | II | 3 | ERC10658.1 | 5416 | 11987 | 550 | ERC10657.1 | 5417 | 11988 | 206 | | | | |
| 1421 | II | 3 | ERE14327.1 | 5419 | 11990 | 550 | ERE14326.1 | 5420 | 11991 | 210 | | | | |
| 1422 | II | 3 | ERC92367.1 | 5422 | 11993 | 550 | ERC92366.1 | 5423 | 11994 | 235 | | | | |
| 1423 | II | 3 | ERD40230.1 | 5425 | 11996 | 550 | ERD40229.1 | 5426 | 11997 | 210 | | | | |
| 1424 | II | 3 | ERC91695.1 | 5428 | 11999 | 550 | ERC91694.1 | 5429 | 12000 | 206 | | | | |
| 1425 | II | 3 | ERD09264.1 | 5431 | 12002 | 550 | ERD09263.1 | 5432 | 12003 | 210 | | | | |
| 1426 | II | 3 | ERC65822.1 | 5434 | 12005 | 550 | ERC65821.1 | 5435 | 12006 | 210 | | | | |
| 1427 | II | 3 | ERC53517.1 | 5437 | 12008 | 550 | ERC53516.1 | 5438 | 12009 | 202 | | | | |
| 1428 | II | 3 | ERE00881.1 | 5440 | 12011 | 550 | ERE00880.1 | 5441 | 12012 | 210 | | | | |
| 1429 | II | 3 | ERE21796.1 | 5443 | 12014 | 550 | ERE21795.1 | 5444 | 12015 | 202 | | | | |
| 1430 | II | 3 | ERC61430.1 | 5446 | 12017 | 550 | ERC61431.1 | 5447 | 12018 | 235 | | | | |
| 1431 | II | 3 | ERC74185.1 | 5449 | 12020 | 550 | ERC74184.1 | 5450 | 12021 | 210 | | | | |
| 1432 | II | 3 | ERC30060.1 | 5452 | 12023 | 550 | ERC30059.1 | 5453 | 12024 | 206 | | | | |
| 1433 | II | 3 | ERC33933.1 | 5455 | 12026 | 550 | ERC33932.1 | 5456 | 12027 | 210 | | | | |
| 1434 | II | 3 | ERC40340.1 | 5458 | 12029 | 550 | ERC40339.1 | 5459 | 12030 | 206 | | | | |
| 1435 | II | 3 | ERC48257.1 | 5461 | 12032 | 550 | ERC48256.1 | 5462 | 12033 | 210 | | | | |
| 1436 | II | 3 | ERD08504.1 | 5464 | 12035 | 550 | ERD08318.1 | 5465 | 12036 | 206 | | | | |
| 1437 | II | 3 | ERD05019.1 | 5467 | 12038 | 550 | ERD05018.1 | 5468 | 12039 | 210 | | | | |
| 1438 | II | 3 | ERD20746.1 | 5470 | 12041 | 550 | ERD20745.1 | 5471 | 12042 | 206 | | | | |
| 1439 | II | 3 | ERD22950.1 | 5473 | 12044 | 550 | ERD22949.1 | 5474 | 12045 | 210 | | | | |
| 1440 | II | 3 | ERD23990.1 | 5476 | 12047 | 550 | ERD23989.1 | 5477 | 12048 | 210 | | | | |
| 1441 | II | 3 | ERD35106.1 | 5479 | 12050 | 550 | ERD35201.1 | 5480 | 12051 | 214 | | | | |
| 1442 | II | 3 | ERD37183.1 | 5482 | 12053 | 550 | ERD37182.1 | 5483 | 12054 | 210 | | | | |
| 1443 | II | 3 | ERD26787.1 | 5485 | 12056 | 498 | ERD26788.1 | 5486 | 12057 | 210 | | | | |
| 1444 | II | 3 | ERD52929.1 | 5488 | 12059 | 550 | ERD52928.1 | 5489 | 12060 | 210 | | | | |
| 1445 | II | 3 | ERD45127.1 | 5491 | 12062 | 550 | ERD45126.1 | 5492 | 12063 | 210 | | | | |
| 1446 | II | 3 | ERD72597.1 | 5494 | 12065 | 550 | ERD72596.1 | 5495 | 12066 | 205 | | | | |
| 1447 | II | 3 | ERD68376.1 | 5497 | 12068 | 550 | ERD68375.1 | 5498 | 12069 | 202 | | | | |
| 1448 | II | 3 | ERD73526.1 | 5500 | 12071 | 498 | ERD73510.1 | 5501 | 12072 | 210 | | | | |
| 1449 | II | 3 | ERD83187.1 | 5503 | 12074 | 550 | ERD83186.1 | 5504 | 12075 | 206 | | | | |
| 1450 | II | 3 | ERD87990.1 | 5506 | 12077 | 550 | ERD87989.1 | 5507 | 12078 | 210 | | | | |
| 1451 | II | 3 | ERD91775.1 | 5509 | 12080 | 550 | ERD91774.1 | 5510 | 12081 | 210 | | | | |
| 1452 | II | 3 | ERD98682.1 | 5512 | 12083 | 550 | ERD98681.1 | 5513 | 12084 | 210 | | | | |
| 1453 | II | 3 | ERD88427.1 | 5515 | 12086 | 498 | ERD88426.1 | 5516 | 12087 | 205 | | | | |
| 1454 | II | 3 | ERE36110.1 | 5518 | 12089 | 550 | ERE36109.1 | 5519 | 12090 | 210 | | | | |
| 1455 | II | 3 | ERE23639.1 | 5521 | 12092 | 550 | ERE23638.1 | 5522 | 12093 | 210 | | | | |
| 1456 | II | 3 | ERE42060.1 | 5524 | 12095 | 550 | ERE42059.1 | 5525 | 12096 | 210 | | | | |
| 1457 | II | 3 | ERE45408.1 | 5527 | 12098 | 550 | ERE45407.1 | 5528 | 12099 | 202 | | | | |
| 1458 | II | 3 | ETJ70764.1 | 5530 | 12101 | 550 | ETJ70765.1 | 5531 | 12102 | 235 | | | | |
| 1459 | II | 3 | ERC07680.1 | 5533 | 12104 | 550 | ERC07679.1 | 5534 | 12105 | 206 | | | | |
| 1460 | II | 3 | ESD10539.1 | 5536 | 12107 | 550 | ESD10538.1 | 5537 | 12108 | 235 | | | | |
| 1461 | II | 3 | ESL20616.1 | 5539 | 12110 | 550 | ESL20617.1 | 5540 | 12111 | 235 | | | | |
| 1462 | II | 3 | ESP12226.1 | 5542 | 12113 | 550 | ESP12225.1 | 5543 | 12114 | 235 | | | | |
| 1463 | II | 3 | ETJ79053.1 | 5545 | 12116 | 550 | ETJ79052.1 | 5546 | 12117 | 235 | | | | |
| 1464 | II | 3 | EST67462.1 | 5548 | 12119 | 550 | EST67463.1 | 5549 | 12120 | 55 | | | | |
| 1465 | II | 3 | EST67376.1 | 5551 | 12122 | 550 | EST67377.1 | 5552 | 12123 | 235 | | | | |
| 1466 | II | 3 | AHG12155.1 | 5554 | 12125 | 498 | AHG12154.1 | 5555 | 12126 | 210 | | | | |
| 1467 | II | 3 | EYE30767.1 | 5557 | 12128 | 550 | EYE30766.1 | 5558 | 12129 | 235 | | | | |
| 1468 | II | 3 | EYE15509.1 | 5560 | 12131 | 550 | EYE15508.1 | 5561 | 12132 | 235 | | | | |
| 1469 | II | 3 | EYE15596.1 | 5563 | 12134 | 550 | EYE15595.1 | 5564 | 12135 | 210 | | | | |
| 1470 | II | 3 | EYW09907.1 | 5566 | 12137 | 550 | EYW09908.1 | 5567 | 12138 | 210 | | | | |
| 1471 | II | 3 | EYW25814.1 | 5569 | 12140 | 550 | EYW25815.1 | 5570 | 12141 | 210 | | | | |
| 1472 | II | 3 | EYW27180.1 | 5572 | 12143 | 550 | EYW27179.1 | 5573 | 12144 | 210 | | | | |
| 1473 | II | 3 | EYW07431.1 | 5575 | 12146 | 550 | EYW07430.1 | 5576 | 12147 | 210 | | | | |
| 1474 | II | 3 | EYW24509.1 | 5578 | 12149 | 550 | EYW24508.1 | 5579 | 12150 | 210 | | | | |
| 1475 | II | 3 | EYW34609.1 | 5581 | 12152 | 550 | EYW34608.1 | 5582 | 12153 | 210 | | | | |
| 1476 | II | 3 | EYW13500.1 | 5584 | 12155 | 550 | EYW13499.1 | 5585 | 12156 | 210 | | | | |
| 1477 | II | 3 | EYV89287.1 | 5587 | 12158 | 550 | EYV89288.1 | 5588 | 12159 | 210 | | | | |
| 1478 | II | 3 | EYY46579.1 | 5590 | 12161 | 550 | EYY46580.1 | 5591 | 12162 | 210 | | | | |
| 1479 | II | 3 | EYV70454.1 | 5593 | 12164 | 550 | EYV70453.1 | 5594 | 12165 | 210 | | | | |
| 1480 | II | 3 | EYV67646.1 | 5596 | 12167 | 550 | EYV67645.1 | 5597 | 12168 | 210 | | | | |
| 1481 | II | 3 | EYV59035.1 | 5599 | 12170 | 550 | EYV59034.1 | 5600 | 12171 | 210 | | | | |
| 1482 | II | 3 | EYV37954.1 | 5602 | 12173 | 550 | EYV37955.1 | 5603 | 12174 | 210 | | | | |
| 1483 | II | 3 | EYV46019.1 | 5605 | 12176 | 550 | EYV46020.1 | 5606 | 12177 | 210 | | | | |
| 1484 | II | 3 | EYV44256.1 | 5608 | 12179 | 550 | EYV44255.1 | 5609 | 12180 | 210 | | | | |
| 1485 | II | 3 | EYV24873.1 | 5611 | 12182 | 550 | EYV24872.1 | 5612 | 12183 | 210 | | | | |
| 1486 | II | 3 | EYV22945.1 | 5614 | 12185 | 550 | EYV22946.1 | 5615 | 12186 | 210 | | | | |
| 1487 | II | 3 | EYV18170.1 | 5617 | 12188 | 550 | EYV18171.1 | 5618 | 12189 | 210 | | | | |
| 1488 | II | 3 | EYV20080.1 | 5620 | 12191 | 550 | EYV20081.1 | 5621 | 12192 | 210 | | | | |
| 1489 | II | 3 | EYV16482.1 | 5623 | 12194 | 550 | EYV16481.1 | 5624 | 12195 | 210 | | | | |
| 1490 | II | 3 | EYW83367.1 | 5626 | 12197 | 550 | EYW83368.1 | 5627 | 12198 | 210 | | | | |
| 1491 | II | 3 | EYW75519.1 | 5629 | 12200 | 550 | EYW75518.1 | 5630 | 12201 | 210 | | | | |
| 1492 | II | 3 | EYW71149.1 | 5632 | 12203 | 550 | EYW71148.1 | 5633 | 12204 | 210 | | | | |
| 1493 | II | 3 | EYW67181.1 | 5635 | 12206 | 550 | EYW67182.1 | 5636 | 12207 | 210 | | | | |
| 1494 | II | 3 | EYW64050.1 | 5638 | 12209 | 550 | EYW64051.1 | 5639 | 12210 | 210 | | | | |
| 1495 | II | 3 | EYW57178.1 | 5641 | 12212 | 550 | EYW57179.1 | 5642 | 12213 | 210 | | | | |
| 1496 | II | 3 | EYW49140.1 | 5644 | 12215 | 550 | EYW49139.1 | 5645 | 12216 | 210 | | | | |
| 1497 | II | 3 | EYW49453.1 | 5647 | 12218 | 550 | EYW49452.1 | 5648 | 12219 | 210 | | | | |
| 1498 | II | 3 | EYW43050.1 | 5650 | 12221 | 550 | EYW43051.1 | 5651 | 12222 | 210 | | | | |
| 1499 | II | 3 | EYW39160.1 | 5653 | 12224 | 550 | EYW39159.1 | 5654 | 12225 | 210 | | | | |
| 1500 | II | 3 | EYX70058.1 | 5656 | 12227 | 550 | EYX70057.1 | 5657 | 12228 | 210 | | | | |
| 1501 | II | 3 | EYX57445.1 | 5659 | 12230 | 550 | EYX57446.1 | 5660 | 12231 | 210 | | | | |
| 1502 | II | 3 | EYX54550.1 | 5662 | 12233 | 550 | EYX54551.1 | 5663 | 12234 | 210 | | | | |
| 1503 | II | 3 | EYX48136.1 | 5665 | 12236 | 550 | EYX48137.1 | 5666 | 12237 | 210 | | | | |
| 1504 | II | 3 | EYX44883.1 | 5668 | 12239 | 550 | EYX44884.1 | 5669 | 12240 | 210 | | | | |
| 1505 | II | 3 | EYX43991.1 | 5671 | 12242 | 550 | EYX43990.1 | 5672 | 12243 | 210 | | | | |
| 1506 | II | 3 | EYX33006.1 | 5674 | 12245 | 550 | EYX33007.1 | 5675 | 12246 | 210 | | | | |
| 1507 | II | 3 | EYX30698.1 | 5677 | 12248 | 550 | EYX30699.1 | 5678 | 12249 | 210 | | | | |
| 1508 | II | 3 | EYX27759.1 | 5680 | 12251 | 550 | EYX27758.1 | 5681 | 12252 | 210 | | | | |
| 1509 | II | 3 | EYX13626.1 | 5683 | 12254 | 550 | EYX13625.1 | 5684 | 12255 | 210 | | | | |
| 1510 | II | 3 | EYX12329.1 | 5686 | 12257 | 550 | EYX12330.1 | 5687 | 12258 | 210 | | | | |
| 1511 | II | 3 | EYX02202.1 | 5689 | 12260 | 550 | EYX02201.1 | 5690 | 12261 | 210 | | | | |
| 1512 | II | 3 | EYW98550.1 | 5692 | 12263 | 550 | EYW98549.1 | 5693 | 12264 | 210 | | | | |
| 1513 | II | 3 | EYW85334.1 | 5695 | 12266 | 550 | EYW85335.1 | 5696 | 12267 | 210 | | | | |
| 1514 | II | 3 | EYV84725.1 | 5698 | 12269 | 550 | EYV84726.1 | 5699 | 12270 | 210 | | | | |
| 1515 | II | 3 | EYZ76664.1 | 5701 | 12272 | 550 | EYZ76665.1 | 5702 | 12273 | 210 | | | | |
| 1516 | II | 3 | EYZ63882.1 | 5704 | 12275 | 550 | EYZ63883.1 | 5705 | 12276 | 210 | | | | |
| 1517 | II | 3 | EYZ52260.1 | 5707 | 12278 | 550 | EYZ52261.1 | 5708 | 12279 | 210 | | | | |
| 1518 | II | 3 | EYZ37414.1 | 5710 | 12281 | 550 | EYZ37413.1 | 5711 | 12282 | 210 | | | | |
| 1519 | II | 3 | EYZ38309.1 | 5713 | 12284 | 550 | EYZ38310.1 | 5714 | 12285 | 210 | | | | |
| 1520 | II | 3 | EYZ36219.1 | 5716 | 12287 | 550 | EYZ36220.1 | 5717 | 12288 | 210 | | | | |
| 1521 | II | 3 | EYZ14531.1 | 5719 | 12290 | 550 | EYZ14532.1 | 5720 | 12291 | 210 | | | | |
| 1522 | II | 3 | EZC28522.1 | 5722 | 12293 | 550 | EZC28521.1 | 5723 | 12294 | 210 | | | | |
| 1523 | II | 3 | EZB48636.1 | 5725 | 12296 | 550 | EZB48635.1 | 5726 | 12297 | 210 | | | | |
| 1524 | II | 3 | EZB05976.1 | 5728 | 12299 | 550 | EZB05977.1 | 5729 | 12300 | 210 | | | | |
| 1525 | II | 3 | EZB18141.1 | 5731 | 12302 | 550 | EZB18140.1 | 5732 | 12303 | 210 | | | | |
| 1526 | II | 3 | EZA89753.1 | 5734 | 12305 | 550 | EZA89754.1 | 5735 | 12306 | 210 | | | | |
| 1527 | II | 3 | EZB32013.1 | 5737 | 12308 | 550 | EZB32012.1 | 5738 | 12309 | 210 | | | | |
| 1528 | II | 3 | EZB20622.1 | 5740 | 12311 | 550 | EZB20623.1 | 5741 | 12312 | 210 | | | | |
| 1529 | II | 3 | EZB41522.1 | 5743 | 12314 | 550 | EZB41521.1 | 5744 | 12315 | 210 | | | | |
| 1530 | II | 3 | EZB11567.1 | 5746 | 12317 | 550 | EZB11568.1 | 5747 | 12318 | 210 | | | | |
| 1531 | II | 3 | EZB46983.1 | 5749 | 12320 | 550 | EZB46982.1 | 5750 | 12321 | 210 | | | | |
| 1532 | II | 3 | EZB04405.1 | 5752 | 12323 | 550 | EZB04406.1 | 5753 | 12324 | 210 | | | | |
| 1533 | II | 3 | EZB74688.1 | 5755 | 12326 | 550 | EZB74687.1 | 5756 | 12327 | 210 | | | | |
| 1534 | II | 3 | EZB73672.1 | 5758 | 12329 | 550 | EZB73671.1 | 5759 | 12330 | 210 | | | | |
| 1535 | II | 3 | EZB73224.1 | 5761 | 12332 | 550 | EZB73225.1 | 5762 | 12333 | 210 | | | | |
| 1536 | II | 3 | EZB87909.1 | 5764 | 12335 | 550 | EZB87908.1 | 5765 | 12336 | 210 | | | | |
| 1537 | II | 3 | EZB80137.1 | 5767 | 12338 | 550 | EZB80138.1 | 5768 | 12339 | 210 | | | | |
| 1538 | II | 3 | EZB81693.1 | 5770 | 12341 | 550 | EZB81692.1 | 5771 | 12342 | 210 | | | | |
| 1539 | II | 3 | EZB96172.1 | 5773 | 12344 | 264 | EZB96173.1 | 5774 | 12345 | 210 | | | | |
| 1540 | II | 3 | EZC15365.1 | 5776 | 12347 | 498 | EZC15364.1 | 5777 | 12348 | 210 | | | | |
| 1541 | II | 3 | EZC19641.1 | 5779 | 12350 | 550 | EZC19640.1 | 5780 | 12351 | 210 | | | | |
| 1542 | II | 3 | EZC20554.1 | 5782 | 12353 | 550 | EZC20553.1 | 5783 | 12354 | 210 | | | | |
| 1543 | II | 3 | EZC30166.1 | 5785 | 12356 | 550 | EZC30167.1 | 5786 | 12357 | 210 | | | | |
| 1544 | II | 3 | EZC37647.1 | 5788 | 12359 | 550 | EZC37648.1 | 5789 | 12360 | 210 | | | | |
| 1545 | II | 3 | EZC43981.1 | 5791 | 12362 | 550 | EZC43980.1 | 5792 | 12363 | 210 | | | | |
| 1546 | II | 3 | EZB95444.1 | 5794 | 12365 | 550 | EZB95445.1 | 5795 | 12366 | 210 | | | | |
| 1547 | II | 3 | EZC48750.1 | 5797 | 12368 | 550 | EZC48751.1 | 5798 | 12369 | 210 | | | | |
| 1548 | II | 3 | EZC62957.1 | 5800 | 12371 | 550 | EZC62958.1 | 5801 | 12372 | 210 | | | | |
| 1549 | II | 3 | EZC70420.1 | 5803 | 12374 | 550 | EZC70419.1 | 5804 | 12375 | 210 | | | | |
| 1550 | II | 3 | EZC77660.1 | 5806 | 12377 | 550 | EZC77659.1 | 5807 | 12378 | 210 | | | | |
| 1551 | II | 3 | EZC64797.1 | 5809 | 12380 | 550 | EZC64798.1 | 5810 | 12381 | 210 | | | | |
| 1552 | II | 3 | EZC90331.1 | 5812 | 12383 | 550 | EZC90330.1 | 5813 | 12384 | 210 | | | | |
| 1553 | II | 3 | EZC92769.1 | 5815 | 12386 | 550 | EZC92768.1 | 5816 | 12387 | 210 | | | | |
| 1554 | II | 3 | EZC86951.1 | 5818 | 12389 | 550 | EZC86950.1 | 5819 | 12390 | 210 | | | | |
| 1555 | II | 3 | EZC83412.1 | 5821 | 12392 | 550 | EZC83411.1 | 5822 | 12393 | 210 | | | | |
| 1556 | II | 3 | EZD75267.1 | 5824 | 12395 | 550 | EZD75266.1 | 5825 | 12396 | 210 | | | | |
| 1557 | II | 3 | EZF08562.1 | 5827 | 12398 | 550 | EZF08563.1 | 5828 | 12399 | 210 | | | | |
| 1558 | II | 3 | EZF05483.1 | 5830 | 12401 | 550 | EZF05484.1 | 5831 | 12402 | 210 | | | | |
| 1559 | II | 3 | EZE61419.1 | 5833 | 12404 | 550 | EZE61418.1 | 5834 | 12405 | 210 | | | | |
| 1560 | II | 3 | EZE88804.1 | 5836 | 12407 | 550 | EZE88803.1 | 5837 | 12408 | 210 | | | | |
| 1561 | II | 3 | EZE89447.1 | 5839 | 12410 | 550 | EZE89446.1 | 5840 | 12411 | 210 | | | | |
| 1562 | II | 3 | EZE90569.1 | 5842 | 12413 | 550 | EZE90570.1 | 5843 | 12414 | 210 | | | | |
| 1563 | II | 3 | EZD08970.1 | 5845 | 12416 | 550 | EZD08971.1 | 5846 | 12417 | 210 | | | | |
| 1564 | II | 3 | EZD16779.1 | 5848 | 12419 | 476 | EZD16778.1 | 5849 | 12420 | 210 | | | | |
| 1565 | II | 3 | EZD15759.1 | 5851 | 12422 | 550 | EZD15760.1 | 5852 | 12423 | 210 | | | | |
| 1566 | II | 3 | EZD19490.1 | 5854 | 12425 | 550 | EZD19491.1 | 5855 | 12426 | 210 | | | | |
| 1567 | II | 3 | EZD00269.1 | 5857 | 12428 | 550 | EZD00270.1 | 5858 | 12429 | 210 | | | | |
| 1568 | II | 3 | EZD85328.1 | 5860 | 12431 | 550 | EZD85327.1 | 5861 | 12432 | 210 | | | | |
| 1569 | II | 3 | EZD90122.1 | 5863 | 12434 | 550 | EZD90121.1 | 5864 | 12435 | 210 | | | | |
| 1570 | II | 3 | EZJ86906.1 | 5866 | 12437 | 550 | EZJ86912.1 | 5867 | 12438 | 205 | | | | |
| 1571 | II | 3 | EZJ76223.1 | 5869 | 12440 | 498 | EZJ76224.1 | 5870 | 12441 | 206 | | | | |
| 1572 | II | 3 | EZJ72758.1 | 5872 | 12443 | 550 | EZJ72757.1 | 5873 | 12444 | 206 | | | | |
| 1573 | II | 3 | EZK00741.1 | 5875 | 12446 | 550 | EZK00742.1 | 5876 | 12447 | 235 | | | | |
| 1574 | II | 3 | EZQ47392.1 | 5878 | 12449 | 550 | EZQ47391.1 | 5879 | 12450 | 210 | | | | |
| 1575 | II | 3 | EZQ50284.1 | 5881 | 12452 | 550 | EZQ50285.1 | 5882 | 12453 | 210 | | | | |
| 1576 | II | 3 | AHY73974.1 | 5884 | 12455 | 498 | AHY73973.1 | 5885 | 12456 | 210 | | | | |
| 1577 | II | 3 | KDA70996.1 | 5887 | 12458 | 550 | KDA70993.1 | 5888 | 12459 | 214 | | | | |
| 1578 | II | 3 | KDA57924.1 | 5890 | 12461 | 498 | KDA57923.1 | 5891 | 12462 | 210 | | | | |
| 1579 | II | 3 | KDM81019.1 | 5893 | 12464 | 550 | KDM81018.1 | 5894 | 12465 | 210 | | | | |
| 1580 | II | 3 | KDM84274.1 | 5896 | 12467 | 550 | KDM84275.1 | 5897 | 12468 | 210 | | | | |
| 1581 | II | 3 | KDT83300.1 | 5899 | 12470 | 550 | KDT83311.1 | 5900 | 12471 | 235 | | | | |
| 1582 | II | 3 | KDX42979.1 | 5902 | 12473 | 498 | KDX42980.1 | 5903 | 12474 | 235 | | | | |
| 1583 | II | 3 | KEJ21097.1 | 5905 | 12476 | 550 | KEJ21096.1 | 5906 | 12477 | 235 | | | | |
| 1584 | II | 3 | KEJ20441.1 | 5908 | 12479 | 550 | KEJ20440.1 | 5909 | 12480 | 210 | | | | |
| 1585 | II | 3 | KEM49610.1 | 5911 | 12482 | 550 | KEM49616.1 | 5912 | 12483 | 210 | | | | |
| 1586 | II | 3 | KEM85726.1 | 5914 | 12485 | 550 | KEM85727.1 | 5915 | 12486 | 210 | | | | |
| 1587 | II | 3 | KEN65095.1 | 5917 | 12488 | 550 | KEN65104.1 | 5918 | 12489 | 210 | | | | |
| 1588 | II | 3 | KEO16490.1 | 5920 | 12491 | 498 | KEO16489.1 | 5921 | 12492 | 235 | | | | |
| 1589 | II | 3 | KEO21427.1 | 5923 | 12494 | 550 | KEO21426.1 | 5924 | 12495 | 235 | | | | |
| 1590 | II | 3 | KEK80772.1 | 5926 | 12497 | 550 | KEK80773.1 | 5927 | 12498 | 235 | | | | |
| 1591 | II | 3 | KEK94609.1 | 5929 | 12500 | 550 | KEK94610.1 | 5930 | 12501 | 235 | | | | |
| 1592 | II | 3 | KEL07350.1 | 5932 | 12503 | 550 | KEL07345.1 | 5933 | 12504 | 206 | | | | |
| 1593 | II | 3 | KEL97937.1 | 5935 | 12506 | 550 | KEL97936.1 | 5936 | 12507 | 235 | | | | |
| 1594 | II | 3 | KEM08518.1 | 5938 | 12509 | 550 | KEM08517.1 | 5939 | 12510 | 235 | | | | |
| 1595 | II | 3 | AIF96932.1 | 5941 | 12512 | 550 | AIF96931.1 | 5942 | 12513 | 235 | | | | |
| 1596 | II | 3 | AIG71772.1 | 5944 | 12515 | 550 | AIG71771.1 | 5945 | 12516 | 205 | | | | |
| 1597 | II | 3 | KHI41604.1 | 5947 | 12518 | 550 | KHI41605.1 | 5948 | 12519 | 235 | | | | |
| 1598 | II | 3 | KHH50139.1 | 5950 | 12521 | 550 | KHH50140.1 | 5951 | 12522 | 235 | | | | |
| 1599 | II | 3 | KHI22225.1 | 5953 | 12524 | 550 | KHI22224.1 | 5954 | 12525 | 235 | | | | |
| 1600 | II | 3 | AJA29434.1 | 5956 | 12527 | 550 | AJA29433.1 | 5957 | 12528 | 235 | | | | |
| 1601 | II | 3 | KIG29572.1 | 5959 | 12530 | 550 | KIG29573.1 | 5960 | 12531 | 235 | | | | |
| 1602 | II | 3 | KIY28271.1 | 5962 | 12533 | 550 | KIY28270.1 | 5963 | 12534 | 235 | | | | |
| 1603 | II | 3 | KIZ09777.1 | 5965 | 12536 | 550 | KIZ09776.1 | 5966 | 12537 | 235 | | | | |
| 1604 | II | 3 | KJJ46592.1 | 5968 | 12539 | 550 | KJJ46591.1 | 5969 | 12540 | 235 | | | | |
| 1605 | II | 3 | KJW55353.1 | 5971 | 12542 | 550 | KJW55352.1 | 5972 | 12543 | 235 | | | | |
| 1606 | II | 3 | KJW51697.1 | 5974 | 12545 | 550 | KJW51698.1 | 5975 | 12546 | 235 | | | | |
| 1607 | II | 3 | KKF77971.1 | 5977 | 12548 | 550 | KKF77970.1 | 5978 | 12549 | 235 | | | | |
| 1608 | II | 3 | KKF80396.1 | 5980 | 12551 | 550 | KKF80395.1 | 5981 | 12552 | 235 | | | | |
| 1609 | II | 3 | KKK30167.1 | 5983 | 12554 | 550 | KKK30168.1 | 5984 | 12555 | 235 | | | | |
| 1610 | II | 3 | KKY44750.1 | 5986 | 12557 | 550 | KKY44749.1 | 5987 | 12558 | 235 | | | | |
| 1611 | II | 3 | KLG52812.1 | 5989 | 12560 | 550 | KLG52813.1 | 5990 | 12561 | 235 | | | | |
| 1612 | II | 3 | KNF16835.1 | 5992 | 12563 | 550 | KNF16834.1 | 5993 | 12564 | 235 | | | | |
| 1613 | II | 3 | KNF20996.1 | 5995 | 12566 | 550 | KNF20997.1 | 5996 | 12567 | 235 | | | | |
| 1614 | II | 3 | KNF39641.1 | 5998 | 12569 | 550 | KNF39642.1 | 5999 | 12570 | 235 | | | | |
| 1615 | II | 3 | KNG40957.1 | 6001 | 12572 | 550 | KNG40956.1 | 6002 | 12573 | 235 | | | | |
| 1616 | II | 3 | CTT73512.1 | 6004 | 12575 | 550 | CTT73497.1 | 6005 | 12576 | 235 | | | | |
| 1617 | II | 3 | KOZ12764.1 | 6007 | 12578 | 550 | KOZ12763.1 | 6008 | 12579 | 235 | | | | |
| 1618 | II | 3 | KOZ62646.1 | 6010 | 12581 | 550 | KOZ62645.1 | 6011 | 12582 | 235 | | | | |
| 1619 | II | 3 | KOZ92032.1 | 6013 | 12584 | 550 | KOZ92033.1 | 6014 | 12585 | 235 | | | | |
| 1620 | II | 3 | KOZ11135.1 | 6016 | 12587 | 550 | KOZ11134.1 | 6017 | 12588 | 235 | | | | |
| 1621 | II | 3 | KOZ14565.1 | 6019 | 12590 | 550 | KOZ14564.1 | 6020 | 12591 | 235 | | | | |
| 1622 | II | 3 | KOZ32808.1 | 6022 | 12593 | 550 | KOZ32807.1 | 6023 | 12594 | 235 | | | | |
| 1623 | II | 3 | KOZ23192.1 | 6025 | 12596 | 550 | KOZ23193.1 | 6026 | 12597 | 235 | | | | |
| 1624 | II | 3 | KOZ33146.1 | 6028 | 12599 | 550 | KOZ33145.1 | 6029 | 12600 | 235 | | | | |
| 1625 | II | 3 | KOZ37735.1 | 6031 | 12602 | 550 | KOZ37734.1 | 6032 | 12603 | 235 | | | | |
| 1626 | II | 3 | KOZ50073.1 | 6034 | 12605 | 550 | KOZ50074.1 | 6035 | 12606 | 235 | | | | |
| 1627 | II | 3 | KOZ47194.1 | 6037 | 12608 | 550 | KOZ47193.1 | 6038 | 12609 | 235 | | | | |
| 1628 | II | 3 | KOZ49646.1 | 6040 | 12611 | 550 | KOZ49647.1 | 6041 | 12612 | 235 | | | | |
| 1629 | II | 3 | KOZ66200.1 | 6043 | 12614 | 550 | KOZ66199.1 | 6044 | 12615 | 235 | | | | |
| 1630 | II | 3 | KOZ69677.1 | 6046 | 12617 | 550 | KOZ69678.1 | 6047 | 12618 | 235 | | | | |
| 1631 | II | 3 | KOZ74922.1 | 6049 | 12620 | 550 | KOZ74921.1 | 6050 | 12621 | 235 | | | | |
| 1632 | II | 3 | KOZ84734.1 | 6052 | 12623 | 550 | KOZ84733.1 | 6053 | 12624 | 235 | | | | |
| 1633 | II | 3 | KOZ80209.1 | 6055 | 12626 | 550 | KOZ80208.1 | 6056 | 12627 | 235 | | | | |
| 1634 | II | 3 | KOZ97678.1 | 6058 | 12629 | 550 | KOZ97679.1 | 6059 | 12630 | 235 | | | | |
| 1635 | II | 3 | KPH45898.1 | 6061 | 12632 | 550 | KPH45897.1 | 6062 | 12633 | 235 | | | | |
| 1636 | II | 3 | KPH35131.1 | 6064 | 12635 | 550 | KPH35132.1 | 6065 | 12636 | 235 | | | | |
| 1637 | II | 3 | KPH38256.1 | 6067 | 12638 | 550 | KPH38255.1 | 6068 | 12639 | 235 | | | | |
| 1638 | II | 3 | ALH93594.1 | 6070 | 12641 | 550 | ALH93593.1 | 6071 | 12642 | 235 | | | | |
| 1639 | II | 3 | KPO74169.1 | 6073 | 12644 | 550 | KPO74168.1 | 6074 | 12645 | 235 | | | | |
| 1640 | II | 3 | KPP03702.1 | 6076 | 12647 | 550 | KPP03703.1 | 6077 | 12648 | 235 | | | | |
| 1641 | II | 3 | KPP08801.1 | 6079 | 12650 | 550 | KPP08802.1 | 6080 | 12651 | 235 | | | | |
| 1642 | II | 3 | KPP18125.1 | 6082 | 12653 | 550 | KPP18126.1 | 6083 | 12654 | 235 | | | | |
| 1643 | II | 3 | KPP23766.1 | 6085 | 12656 | 550 | KPP23765.1 | 6086 | 12657 | 235 | | | | |
| 1644 | II | 3 | KPP34280.1 | 6088 | 12659 | 550 | KPP34279.1 | 6089 | 12660 | 235 | | | | |
| 1645 | II | 3 | KPP38850.1 | 6091 | 12662 | 550 | KPP38849.1 | 6092 | 12663 | 235 | | | | |
| 1646 | II | 3 | KPP49038.1 | 6094 | 12665 | 550 | KPP49037.1 | 6095 | 12666 | 235 | | | | |
| 1647 | II | 3 | KPP10066.1 | 6097 | 12668 | 550 | KPP10065.1 | 6098 | 12669 | 235 | | | | |
| 1648 | II | 3 | KPP41788.1 | 6100 | 12671 | 550 | KPP41787.1 | 6101 | 12672 | 235 | | | | |
| 1649 | II | 3 | KPP53631.1 | 6103 | 12674 | 550 | KPP53632.1 | 6104 | 12675 | 235 | | | | |
| 1650 | II | 3 | KRQ04754.1 | 6106 | 12677 | 550 | KRQ04753.1 | 6107 | 12678 | 235 | | | | |
| 1651 | II | 3 | KUG91604.1 | 6109 | 12680 | 550 | KUG91603.1 | 6110 | 12681 | 235 | | | | |
| 1652 | II | 3 | KUH02053.1 | 6112 | 12683 | 550 | KUH02054.1 | 6113 | 12684 | 235 | | | | |
| 1653 | II | 3 | KUS40191.1 | 6115 | 12686 | 550 | KUS40190.1 | 6116 | 12687 | 235 | | | | |
| 1654 | II | 3 | KUT25846.1 | 6118 | 12689 | 550 | KUT25845.1 | 6119 | 12690 | 235 | | | | |
| 1655 | II | 3 | KUT16910.1 | 6121 | 12692 | 550 | KUT16911.1 | 6122 | 12693 | 235 | | | | |
| 1656 | II | 3 | KUT50286.1 | 6124 | 12695 | 550 | KUT50285.1 | 6125 | 12696 | 235 | | | | |
| 1657 | II | 3 | KUU89821.1 | 6127 | 12698 | 550 | KUU89822.1 | 6128 | 12699 | 235 | | | | |
| 1658 | II | 3 | KUT63217.1 | 6130 | 12701 | 550 | KUT63218.1 | 6131 | 12702 | 235 | | | | |
| 1659 | II | 3 | KUU32866.1 | 6133 | 12704 | 550 | KUU32867.1 | 6134 | 12705 | 235 | | | | |
| 1660 | II | 3 | KUU24010.1 | 6136 | 12707 | 550 | KUU24009.1 | 6137 | 12708 | 235 | | | | |
| 1661 | II | 3 | KUV87991.1 | 6139 | 12710 | 550 | KUV87990.1 | 6140 | 12711 | 235 | | | | |
| 1662 | II | 3 | KUW19155.1 | 6142 | 12713 | 550 | KUW19156.1 | 6143 | 12714 | 235 | | | | |
| 1663 | II | 3 | KUW69106.1 | 6145 | 12716 | 550 | KUW69107.1 | 6146 | 12717 | 235 | | | | |
| 1664 | II | 3 | KUW47131.1 | 6148 | 12719 | 550 | KUW47132.1 | 6149 | 12720 | 235 | | | | |
| 1665 | II | 3 | AMG80376.1 | 6151 | 12722 | 550 | AMG80377.1 | 6152 | 12723 | 235 | | | | |
| 1666 | II | 3 | KXK90272.1 | 6154 | 12725 | 550 | KXK90273.1 | 6155 | 12726 | 235 | | | | |
| 1667 | II | 3 | KXP75340.1 | 6157 | 12728 | 550 | KXP75341.1 | 6158 | 12729 | 235 | | | | |
| 1668 | II | 3 | KXR06383.1 | 6160 | 12731 | 550 | KXR06382.1 | 6161 | 12732 | 235 | | | | |
| 1669 | II | 3 | KXR86340.1 | 6163 | 12734 | 550 | KXR86339.1 | 6164 | 12735 | 235 | | | | |
| 1670 | II | 3 | EYT05881.1 | 6166 | 12737 | 550 | EYT05880.1 | 6167 | 12738 | 235 | | | | |
| 1671 | II | 3 | KMV95472.1 | 6169 | 12740 | 550 | KMV95471.1 | 6170 | 12741 | 235 | | | | |
| 1672 | II | 3 | ESN46751.1 | 6172 | 12743 | 550 | ESN46750.1 | 6173 | 12744 | 235 | | | | |
| 1673 | II | 3 | KDL89510.1 | 6175 | 12746 | 550 | KDL89509.1 | 6176 | 12747 | 235 | | | | |
| 1674 | II | 3 | KMG65747.1 | 6178 | 12749 | 550 | KMG65746.1 | 6179 | 12750 | 235 | | | | |
| 1675 | II | 3 | KRR31030.1 | 6181 | 12752 | 550 | KRR31031.1 | 6182 | 12753 | 235 | | | | |
| 1676 | II | 3 | CVD67996.1 | 6184 | 12755 | 550 | CVD68041.1 | 6185 | 12756 | 205 | | | | |
| 1677 | II | 3 | CVD62450.1 | 6187 | 12758 | 550 | CVD62420.1 | 6188 | 12759 | 205 | | | | |
| 1678 | II | 3 | CVA98419.1 | 6190 | 12761 | 550 | CVA98397.1 | 6191 | 12762 | 205 | | | | |
| 1679 | II | 3 | CVB67027.1 | 6193 | 12764 | 550 | CVB66985.1 | 6194 | 12765 | 205 | | | | |
| 1680 | II | 3 | CVE07719.1 | 6196 | 12767 | 550 | CVE07671.1 | 6197 | 12768 | 205 | | | | |
| 1681 | II | 3 | CUZ34178.1 | 6199 | 12770 | 550 | CUZ34215.1 | 6200 | 12771 | 205 | | | | |
| 1682 | II | 3 | CVH03780.1 | 6202 | 12773 | 550 | CVH03759.1 | 6203 | 12774 | 205 | | | | |
| 1683 | II | 3 | CUZ32573.1 | 6205 | 12776 | 550 | CUZ32642.1 | 6206 | 12777 | 205 | | | | |
| 1684 | II | 3 | CVC01626.1 | 6208 | 12779 | 550 | CVC01593.1 | 6209 | 12780 | 205 | | | | |
| 1685 | II | 3 | CVC86950.1 | 6211 | 12782 | 550 | CVC86922.1 | 6212 | 12783 | 205 | | | | |
| 1686 | II | 3 | CVC74574.1 | 6214 | 12785 | 550 | CVC74549.1 | 6215 | 12786 | 205 | | | | |
| 1687 | II | 3 | CVC09129.1 | 6217 | 12788 | 550 | CVC09161.1 | 6218 | 12789 | 205 | | | | |
| 1688 | II | 3 | CVG04189.1 | 6220 | 12791 | 550 | CVG04225.1 | 6221 | 12792 | 205 | | | | |
| 1689 | II | 3 | CVB23886.1 | 6223 | 12794 | 550 | CVB23925.1 | 6224 | 12795 | 205 | | | | |
| 1690 | II | 3 | CVE95450.1 | 6226 | 12797 | 550 | CVE95488.1 | 6227 | 12798 | 205 | | | | |
| 1691 | II | 3 | CVG92218.1 | 6229 | 12800 | 550 | CVG92173.1 | 6230 | 12801 | 205 | | | | |
| 1692 | II | 3 | CVA05211.1 | 6232 | 12803 | 550 | CVA05242.1 | 6233 | 12804 | 205 | | | | |
| 1693 | II | 3 | CQJ21079.1 | 6235 | 12806 | 550 | CQJ21085.1 | 6236 | 12807 | 235 | | | | |
| 1694 | II | 3 | CNK85706.1 | 6238 | 12809 | 546 | CNK85680.1 | 6239 | 12810 | 235 | | | | |
| 1695 | II | 3 | KLE23459.1 | 6241 | 12812 | 590 | KLE23458.1 | 6242 | 12813 | 235 | | | | |
| 1696 | II | 3 | KNY42369.1 | 6244 | 12815 | 583 | KNY42368.1 | 6245 | 12816 | 235 | | | | |
| 1697 | II | 3 | KTL44731.1 | 6247 | 12818 | 583 | KTL44732.1 | 6248 | 12819 | 235 | | | | |
| 1698 | II | 3 | KED68169.1 | 6250 | 12821 | 583 | KED68170.1 | 6251 | 12822 | 235 | | | | |
| 1699 | II | 3 | KHF16016.1 | 6253 | 12824 | 583 | KHF16015.1 | 6254 | 12825 | 235 | | | | |
| 1700 | II | 3 | KIT58956.1 | 6256 | 12827 | 486 | KIT58955.1 | 6257 | 12828 | 235 | | | | |
| 1701 | II | 3 | KIT38898.1 | 6259 | 12830 | 486 | KIT38899.1 | 6260 | 12831 | 225 | | | | |
| 1702 | II | 3 | KJR33562.1 | 6262 | 12833 | 583 | KJR33561.1 | 6263 | 12834 | 235 | | | | |
| 1703 | II | 3 | KKX83991.1 | 6265 | 12836 | 583 | KKX83990.1 | 6266 | 12837 | 235 | | | | |
| 1704 | II | 3 | KOE91954.1 | 6268 | 12839 | 583 | KOE91953.1 | 6269 | 12840 | 235 | | | | |
| 1705 | II | 3 | KOF38061.1 | 6271 | 12842 | 583 | KOF38060.1 | 6272 | 12843 | 235 | | | | |
| 1706 | II | 3 | KON59226.1 | 6274 | 12845 | 515 | KON59157.1 | 6275 | 12846 | 237 | | | | |
| 1707 | II | 3 | KXG05869.1 | 6277 | 12848 | 583 | KXG05868.1 | 6278 | 12849 | 235 | | | | |
| 1708 | II | 3 | KYJ92316.1 | 6280 | 12851 | 583 | KYJ92315.1 | 6281 | 12852 | 235 | | | | |
| 1709 | II | 3 | EMB49752.1 | 6283 | 12854 | 414 | EMB49753.1 | 6284 | 12855 | 235 | | | | |
| 1710 | II | 3 | EDL69253.1 | 6286 | 12857 | 583 | EDL69242.1 | 6287 | 12858 | 235 | | | | |
| 1711 | II | 3 | EFM85262.1 | 6289 | 12860 | 552 | EFM85263.1 | 6290 | 12861 | 230 | | | | |
| 1712 | II | 3 | EFM93934.1 | 6292 | 12863 | 552 | EFM93935.1 | 6293 | 12864 | 230 | | | | |
| 1713 | II | 3 | EFM98321.1 | 6295 | 12866 | 552 | EFM98322.1 | 6296 | 12867 | 230 | | | | |
| 1714 | II | 3 | ABI24728.1 | 6298 | 12869 | 589 | ABI24729.1 | 6299 | 12870 | 229 | | | | |
| 1715 | II | 3 | EYA48173.1 | 6301 | 12872 | 529 | EYA48172.1 | 6302 | 12873 | 233 | | | | |
| 1716 | II | 3 | EYA75808.1 | 6304 | 12875 | 529 | EYA75712.1 | 6305 | 12876 | 233 | | | | |
| 1717 | II | 3 | EYA68803.1 | 6307 | 12878 | 527 | EYA68804.1 | 6308 | 12879 | 233 | | | | |
| 1718 | II | 3 | EYA80659.1 | 6310 | 12881 | 529 | EYA80658.1 | 6311 | 12882 | 233 | | | | |
| 1719 | II | 3 | CUA18412.1 | 6313 | 12884 | 529 | CUA18411.1 | 6314 | 12885 | 233 | | | | |
| 1720 | II | 3 | KDS43041.1 | 6316 | 12887 | 515 | KDS43042.1 | 6317 | 12888 | 233 | | | | |
| 1721 | II | 3 | KDS37991.1 | 6319 | 12890 | 515 | KDS37899.1 | 6320 | 12891 | 233 | | | | |
| 1722 | II | 3 | KDS73983.1 | 6322 | 12893 | 515 | KDS73984.1 | 6323 | 12894 | 233 | | | | |
| 1723 | II | 3 | ALF18255.1 | 6325 | 12896 | 643 | ALF18256.1 | 6326 | 12897 | 234 | | | | |
| 1724 | II | 3 | KXA24061.1 | 6328 | 12899 | 643 | KXA24060.1 | 6329 | 12900 | 234 | | | | |
| 1725 | II | 3 | EFG28532.1 | 6331 | 12902 | 711 | EFG28533.1 | 6332 | 12903 | 232 | | | | |
| 1726 | II | 3 | ADJ62020.1 | 6334 | 12905 | 435 | ADJ62019.1 | 6335 | 12906 | 232 | | | | |
| 1727 | II | 3 | KTC70124.1 | 6337 | 12908 | 498 | KTC70123.1 | 6338 | 12909 | 230 | | | | |
| 1728 | II | 3 | AFT94972.1 | 6340 | 12911 | 598 | AFT94973.1 | 6341 | 12912 | 235 | | | | |
| 1729 | II | 3 | EEX38485.1 | 6343 | 12914 | 583 | EEX38486.1 | 6344 | 12915 | 235 | | | | |
| 1730 | II | 3 | CCN78625.1 | 6346 | 12917 | 592 | CCN78626.1 | 6347 | 12918 | 236 | | | | |
| 1731 | II | 3 | CCN34617.1 | 6349 | 12920 | 592 | CCN34618.1 | 6350 | 12921 | 236 | | | | |
| 1732 | II | 3 | CCN40571.1 | 6352 | 12923 | 592 | CCN40570.1 | 6353 | 12924 | 236 | | | | |
| 1733 | II | 3 | CCN66135.1 | 6355 | 12926 | 592 | CCN66136.1 | 6356 | 12927 | 236 | | | | |
| 1734 | II | 3 | KGJ97945.1 | 6358 | 12929 | 598 | KGJ97946.1 | 6359 | 12930 | 238 | | | | |
| 1735 | II | 3 | EEP49046.1 | 6361 | 12932 | 435 | EEP51863.1 | 6362 | 12933 | 230 | | | | |
| 1736 | II | 3 | AIP07406.1 | 6364 | 12935 | 435 | AIP07455.1 | 6365 | 12936 | 230 | | | | |
| 1737 | II | 3 | KGC46655.1 | 6367 | 12938 | 435 | KGC46770.1 | 6368 | 12939 | 230 | | | | |
| 1738 | II | 3 | AIV54526.1 | 6370 | 12941 | 435 | AIV54849.1 | 6371 | 12942 | 230 | | | | |
| 1739 | II | 3 | KGU70585.1 | 6373 | 12944 | 435 | KGU70576.1 | 6374 | 12945 | 230 | | | | |
| 1740 | II | 3 | KGV18629.1 | 6376 | 12947 | 435 | KGV17582.1 | 6377 | 12948 | 230 | | | | |
| 1741 | II | 3 | KGV95069.1 | 6379 | 12950 | 435 | KGV94752.1 | 6380 | 12951 | 230 | | | | |
| 1742 | II | 3 | KGV65245.1 | 6382 | 12953 | 435 | KGV65979.1 | 6383 | 12954 | 230 | | | | |
| 1743 | II | 3 | KGV93341.1 | 6385 | 12956 | 435 | KGV92723.1 | 6386 | 12957 | 230 | | | | |
| 1744 | II | 3 | KGX53114.1 | 6388 | 12959 | 435 | KGX53127.1 | 6389 | 12960 | 230 | | | | |
| 1745 | II | 3 | KGX64948.1 | 6391 | 12962 | 435 | KGX64947.1 | 6392 | 12963 | 230 | | | | |
| 1746 | II | 3 | AJX70756.1 | 6394 | 12965 | 435 | AJX71777.1 | 6395 | 12966 | 230 | | | | |
| 1747 | II | 3 | AIA69103.1 | 6397 | 12968 | 459 | AIA69102.1 | 6398 | 12969 | 244 | | | | |
| 1748 | II | 3 | AIK12008.1 | 6400 | 12971 | 459 | AIK12007.1 | 6401 | 12972 | 244 | | | | |
| 1749 | II | 3 | KMK83438.1 | 6403 | 12974 | 459 | KMK83437.1 | 6404 | 12975 | 244 | | | | |
| 1750 | II | 3 | KGK11299.1 | 6406 | 12977 | 583 | KGK11298.1 | 6407 | 12978 | 235 | | | | |
| 1751 | II | 3 | EAR22192.1 | 6409 | 12980 | 606 | EAR22191.1 | 6410 | 12981 | 237 | | | | |
| 1752 | II | 3 | ERG19017.1 | 6412 | 12983 | 391 | ERG19018.1 | 6413 | 12984 | 217 | | | | |
| 1753 | II | 3 | ERG19053.1 | 6415 | 12986 | 599 | ERG19054.1 | 6416 | 12987 | 235 | | | | |
| 1754 | II | 3 | ERG37343.1 | 6418 | 12989 | 588 | ERG37344.1 | 6419 | 12990 | 234 | | | | |
| 1755 | II | 3 | KTD25444.1 | 6421 | 12992 | 494 | KTD25443.1 | 6422 | 12993 | 230 | | | | |
| 1756 | II | 3 | EER46085.1 | 6424 | 12995 | 586 | EER46086.1 | 6425 | 12996 | 229 | | | | |
| 1757 | II | 3 | AKZ71842.1 | 6427 | 12998 | 550 | AKZ71841.1 | 6428 | 12999 | 235 | | | | |
| 1758 | II | 3 | KJN72848.1 | 6430 | 13001 | 550 | KJN72849.1 | 6431 | 13002 | 235 | | | | |
| 1759 | II | 3 | KLQ05988.1 | 6433 | 13004 | 550 | KLQ05987.1 | 6434 | 13005 | 235 | | | | |
| 1760 | II | 3 | KHE02046.1 | 6436 | 13007 | 433 | KHE02111.1 | 6437 | 13008 | 226 | | | | |
| 1761 | II | 3 | KHN64404.1 | 6439 | 13010 | 433 | KHN64740.1 | 6440 | 13011 | 226 | | | | |
| 1762 | II | 3 | ACS83827.1 | 6442 | 13013 | 520 | ACS83826.1 | 6443 | 13014 | 248 | | | | |
| 1763 | II | 3 | EAS45749.1 | 6445 | 13016 | 585 | EAS45750.1 | 6446 | 13017 | 237 | | | | |
| 1764 | II | 3 | ALP65206.1 | 6448 | 13019 | 435 | ALP66609.1 | 6449 | 13020 | 230 | | | | |
| 1765 | II | 3 | AIO36173.1 | 6451 | 13022 | 445 | AIO34939.1 | 6452 | 13023 | 230 | | | | |
| 1766 | II | 3 | KTC93828.1 | 6454 | 13025 | 494 | KTC93827.1 | 6455 | 13026 | 230 | | | | |
| 1767 | II | 3 | KVP70159.1 | 6457 | 13028 | 435 | KVP70160.1 | 6458 | 13029 | 232 | | | | |
| 1768 | II | 3 | KVP49168.1 | 6460 | 13031 | 435 | KVP49169.1 | 6461 | 13032 | 232 | | | | |
| 1769 | II | 3 | KVQ79696.1 | 6463 | 13034 | 435 | KVQ79697.1 | 6464 | 13035 | 232 | | | | |
| 1770 | II | 3 | KVR10907.1 | 6466 | 13037 | 435 | KVR10908.1 | 6467 | 13038 | 232 | | | | |
| 1771 | II | 3 | KVR50011.1 | 6469 | 13040 | 435 | KVR50012.1 | 6470 | 13041 | 232 | | | | |
| 1772 | II | 3 | KVR54358.1 | 6472 | 13043 | 435 | KVR54359.1 | 6473 | 13044 | 232 | | | | |
| 1773 | II | 3 | KWB45018.1 | 6475 | 13046 | 435 | KWB45017.1 | 6476 | 13047 | 232 | | | | |
| 1774 | II | 3 | KWB78076.1 | 6478 | 13049 | 435 | KWB78077.1 | 6479 | 13050 | 232 | | | | |
| 1775 | II | 3 | KWD39079.1 | 6481 | 13052 | 435 | KWD39080.1 | 6482 | 13053 | 232 | | | | |
| 1776 | II | 3 | KWD33064.1 | 6484 | 13055 | 435 | KWD33065.1 | 6485 | 13056 | 232 | | | | |
| 1777 | II | 3 | KWD38753.1 | 6487 | 13058 | 435 | KWD38754.1 | 6488 | 13059 | 232 | | | | |
| 1778 | II | 3 | KWO93445.1 | 6490 | 13061 | 435 | KWO93444.1 | 6491 | 13062 | 232 | | | | |
| 1779 | II | 3 | KVX24372.1 | 6493 | 13064 | 435 | KVX24373.1 | 6494 | 13065 | 232 | | | | |
| 1780 | II | 3 | KGO04268.1 | 6496 | 13067 | 488 | KGO04269.1 | 6497 | 13068 | 233 | | | | |
| 1781 | II | 3 | ADE14195.1 | 6499 | 13070 | 606 | ADE14194.1 | 6500 | 13071 | 237 | | | | |
| 1782 | II | 3 | ADR35021.1 | 6502 | 13073 | 378 | ADR35022.1 | 6503 | 13074 | 238 | | | | |
| 1783 | II | 3 | EPF16739.1 | 6505 | 13076 | 435 | EPF16738.1 | 6506 | 13077 | 226 | | | | |
| 1784 | II | 3 | KYL32886.1 | 6508 | 13079 | 587 | KYL32887.1 | 6509 | 13080 | 234 | | | | |
| 1785 | II | 3 | ALS34707.1 | 6511 | 13082 | 588 | ALS34708.1 | 6512 | 13083 | 232 | | | | |
| 1786 | II | 3 | KOO13437.1 | 6514 | 13085 | 583 | KOO13436.1 | 6515 | 13086 | 235 | | | | |
| 1787 | II | 3 | KGA93591.1 | 6517 | 13088 | 387 | KGA93590.1 | 6518 | 13089 | 238 | | | | |
| 1788 | II | 3 | CCK76631.1 | 6520 | 13091 | 572 | CCK76632.1 | 6521 | 13092 | 234 | | | | |
| 1789 | II | 3 | CAV18530.1 | 6523 | 13094 | 590 | CAV18531.1 | 6524 | 13095 | 236 | | | | |
| 1790 | II | 3 | CEO93970.1 | 6526 | 13097 | 490 | CEO93972.1 | 6527 | 13098 | 196 | | | | |
| 1791 | II | 3 | CDT36105.1 | 6529 | 13100 | 567 | CDT36097.1 | 6530 | 13101 | 233 | | | | |
| 1792 | II | 3 | CDT66931.1 | 6532 | 13103 | 567 | CDT66922.1 | 6533 | 13104 | 233 | | | | |
| 1793 | II | 3 | EIY57316.1 | 6535 | 13106 | 529 | EIY57317.1 | 6536 | 13107 | 233 | | | | |
| 1794 | II | 3 | EPR32275.1 | 6538 | 13109 | 439 | EPR32276.1 | 6539 | 13110 | 235 | | | | |
| 1795 | II | 3 | ENO91777.1 | 6541 | 13112 | 532 | ENO91778.1 | 6542 | 13113 | 243 | | | | |
| 1796 | II | 3 | EAQ53529.1 | 6544 | 13115 | 519 | EAQ53528.1 | 6545 | 13116 | 236 | | | | |
| 1797 | II | 3 | GAA81805.1 | 6547 | 13118 | 588 | GAA81804.1 | 6548 | 13119 | 232 | | | | |
| 1798 | II | 3 | EEF80486.1 | 6550 | 13121 | 599 | EEF80469.1 | 6551 | 13122 | 212 | | | | |
| 1799 | II | 3 | KVA10031.1 | 6553 | 13124 | 435 | KVA10030.1 | 6554 | 13125 | 230 | | | | |
| 1800 | II | 3 | GAD76289.1 | 6556 | 13127 | 583 | GAD76290.1 | 6557 | 13128 | 235 | | | | |
| 1801 | II | 3 | EFM21230.1 | 6559 | 13130 | 550 | EFM21229.1 | 6560 | 13131 | 235 | | | | |
| 1802 | II | 3 | KJF90748.1 | 6562 | 13133 | 583 | KJF90749.1 | 6563 | 13134 | 235 | | | | |
| 1803 | II | 3 | KKB47519.1 | 6565 | 13136 | 508 | KKB47518.1 | 6566 | 13137 | 233 | | | | |
| 1804 | II | 3 | EIM72585.1 | 6568 | 13139 | 289 | EIM72584.1 | 6569 | 13140 | 249 | | | | |
| 1805 | II | 3 | EEX64653.1 | 6571 | 13142 | 414 | EEX64652.1 | 6572 | 13143 | 217 | | | | |
| 1806 | II | 3 | EEY99587.1 | 6574 | 13145 | 584 | EEY99586.1 | 6575 | 13146 | 217 | | | | |
| 1807 | II | 3 | EGW44491.1 | 6577 | 13148 | 425 | EGW44490.1 | 6578 | 13149 | 236 | | | | |
| 1808 | II | 3 | KHD25429.1 | 6580 | 13151 | 679 | KHD25430.1 | 6581 | 13152 | 236 | | | | |
| 1809 | II | 3 | GAL03971.1 | 6583 | 13154 | 658 | GAL03972.1 | 6584 | 13155 | 235 | | | | |
| 1810 | II | 3 | ELA08940.1 | 6586 | 13157 | 561 | ELA08939.1 | 6587 | 13158 | 230 | | | | |
| 1811 | II | 3 | AEH01975.1 | 6589 | 13160 | 407 | AEH01976.1 | 6590 | 13161 | 234 | | | | |
| 1812 | II | 3 | EKM24778.1 | 6592 | 13163 | 591 | EKM24779.1 | 6593 | 13164 | 236 | | | | |
| 1813 | II | 3 | EOD77073.1 | 6595 | 13166 | 583 | EOD77074.1 | 6596 | 13167 | 236 | | | | |
| 1814 | II | 3 | KEQ18612.1 | 6598 | 13169 | 675 | KEQ18611.1 | 6599 | 13170 | 234 | | | | |
| 1815 | II | 3 | EJF07133.1 | 6601 | 13172 | 511 | EJF07137.1 | 6602 | 13173 | 245 | | | | |
| 1816 | II | 3 | KWU01629.1 | 6604 | 13175 | 677 | KWU01630.1 | 6605 | 13176 | 236 | | | | |
| 1817 | II | 3 | KHL11033.1 | 6607 | 13178 | 435 | KHL11032.1 | 6608 | 13179 | 230 | | | | |
| 1818 | II | 3 | AIL32429.1 | 6610 | 13181 | 506 | AIL32428.1 | 6611 | 13182 | 222 | | | | |
| 1819 | II | 3 | KIM02579.1 | 6613 | 13184 | 399 | KIM02580.1 | 6614 | 13185 | 230 | | | | |
| 1820 | II | 3 | KIM12269.1 | 6616 | 13187 | 440 | KIM12268.1 | 6617 | 13188 | 250 | | | | |
| 1821 | II | 3 | EJU08777.1 | 6619 | 13190 | 659 | EJU08778.1 | 6620 | 13191 | 234 | | | | |
| 1822 | II | 3 | AJA69892.1 | 6622 | 13193 | 463 | AJA69893.1 | 6623 | 13194 | 235 | | | | |
| 1823 | II | 3 | KRG22676.1 | 6625 | 13196 | 497 | KRG22677.1 | 6626 | 13197 | 214 | | | | |
| 1824 | II | 3 | KLN64107.1 | 6628 | 13199 | 550 | KLN64108.1 | 6629 | 13200 | 235 | | | | |
| 1825 | II | 3 | KDM93486.1 | 6631 | 13202 | 560 | KDM93485.1 | 6632 | 13203 | 235 | | | | |
| 1826 | II | 3 | KLE39938.1 | 6634 | 13205 | 550 | KLE39937.1 | 6635 | 13206 | 235 | | | | |
| 1827 | II | 3 | KPZ54442.1 | 6637 | 13208 | 588 | KPZ54443.1 | 6638 | 13209 | 232 | | | | |
| 1828 | II | 3 | KQN43371.1 | 6640 | 13211 | 547 | KQN43544.1 | 6641 | 13212 | 240 | | | | |
| 1829 | II | 3 | CVJ63610.1 | 6643 | 13214 | 435 | CVJ63623.1 | 6644 | 13215 | 230 | | | | |
| 1830 | II | 3 | CVE17086.1 | 6646 | 13217 | 550 | CVE17042.1 | 6647 | 13218 | 205 | | | | |

(Part C)

| | | | | Y | | | | AC | | | |
|---|---|---|---|---|---|---|---|---|---|---|---|
| | | | | ZorD | | | | ZorE | | | |
| | V | W | X | protein | Z | AA | AB | protein | AD | AE | |
| | ZorD | PRT SEQ | DNA SEQ | length | ZorE | PRT SEQ | DNA SEQ | length | Genomic Start | Genomic End | AF |
| 1 | protein_accession | ID NO: | ID NO: | (aa) | protein_accession | ID NO: | ID NO: | (aa) | Point | Point | comments |
| 2 | | | | | | | | | 1103024 | 1107464 | |
| 3 | KGH31557.1 | 84 | 6655 | 1089 | | | | | 109913 | 117783 | |
| 4 | ABR83422.1 | 88 | 6659 | 1010 | | | | | 6232483 | 6240424 | |
| 5 | EIE46560.1 | 92 | 6663 | 1013 | | | | | 167614 | 175555 | |
| 6 | EKA35838.1 | 96 | 6667 | 1083 | | | | | 52101 | 59974 | |
| 7 | EKA51670.1 | 100 | 6671 | 1083 | | | | | 3200 | 11072 | |
| 8 | EOQ78911.1 | 104 | 6675 | 1083 | | | | | 9650 | 17440 | |
| 9 | | | | | | | | | 4597 | 8758 | |
| 10 | ERY08920.1 | 111 | 6682 | 1013 | | | | | 714819 | 722760 | |
| 11 | ERY16787.1 | 115 | 6686 | 1083 | | | | | 5165403 | 5173276 | |
| 12 | ERY21331.1 | 119 | 6690 | 1013 | | | | | 1199237 | 1207178 | |
| 13 | ERV56554.1 | 123 | 6694 | 1013 | | | | | 739445 | 747386 | |
| 14 | ERX20608.1 | 127 | 6698 | 1083 | | | | | 1361333 | 1369206 | |
| 15 | ERX27341.1 | 131 | 6702 | 1083 | | | | | 19893 | 27765 | |
| 16 | ERZ22538.1 | 135 | 6706 | 1083 | | | | | 2803767 | 2811640 | |
| 17 | ERX75593.1 | 139 | 6710 | 1083 | | | | | 1559729 | 1567601 | |
| 18 | ESR70189.1 | 143 | 6714 | 1083 | | | | | 64238 | 72111 | |
| 19 | ETU86356.1 | 147 | 6718 | 1013 | | | | | 956562 | 964503 | |
| 20 | CDI94245.1 | 151 | 6722 | 1028 | | | | | 6519543 | 6527528 | Additional ZorC homolog CDI94246.1 |
| 21 | CDI95169.1 | 155 | 6726 | 1083 | | | | | 7388834 | 7396675 | |
| 22 | EVT82479.1 | 159 | 6730 | 782 | | | | | 1 | 7247 | |
| 23 | EYU03500.1 | 163 | 6734 | 1082 | | | | | 54121 | 61990 | |
| 24 | EZN45281.1 | 167 | 6738 | 1009 | | | | | 8398 | 16691 | |
| 25 | EZO27047.1 | 171 | 6742 | 1014 | | | | | 504264 | 512118 | |
| 26 | EZP05711.1 | 175 | 6746 | 1014 | | | | | 1929997 | 1937851 | |
| 27 | CDH74039.1 | 179 | 6750 | 1028 | | | | | 47360 | 55346 | |
| 28 | CDH70664.1 | 183 | 6754 | 1083 | | | | | 20547 | 28388 | |
| 29 | KEA10738.1 | 187 | 6758 | 1013 | | | | | 116482 | 124423 | |
| 30 | AJF50274.1 | 191 | 6762 | 1083 | | | | | 1683969 | 1691810 | |
| 31 | AKE69363.1 | 195 | 6766 | 1083 | | | | | 2967762 | 2975603 | |
| 32 | AKE72975.1 | 199 | 6770 | 1028 | | | | | 7120178 | 7128164 | |
| 33 | CRQ30613.1 | 203 | 6774 | 1028 | | | | | 10264 | 18250 | |
| 34 | CRQ14077.1 | 207 | 6778 | 1083 | | | | | 20601 | 28442 | |
| 35 | CRQ20861.1 | 211 | 6782 | 1028 | | | | | 21948 | 29934 | |
| 36 | CRP04479.1 | 215 | 6786 | 1083 | | | | | 2221 | 10062 | |
| 37 | CRQ05508.1 | 219 | 6790 | 1083 | | | | | 20602 | 28443 | |
| 38 | CRP93982.1 | 223 | 6794 | 1028 | | | | | 14375 | 22361 | |
| 39 | CRP94802.1 | 227 | 6798 | 1028 | | | | | 21977 | 29963 | |
| 40 | CRQ51017.1 | 231 | 6802 | 1083 | | | | | 7536 | 15377 | |
| 41 | CRS01084.1 | 235 | 6806 | 1028 | | | | | 19796 | 27782 | |
| 42 | CRX34597.1 | 239 | 6810 | 1028 | | | | | 21981 | 29967 | |
| 43 | CRX35808.1 | 243 | 6814 | 1083 | | | | | 7536 | 15377 | |
| 44 | CRN87050.1 | 247 | 6818 | 1083 | | | | | 7536 | 15377 | |
| 45 | CRQ28286.1 | 251 | 6822 | 1083 | | | | | 20602 | 28443 | |
| 46 | CRQ21590.1 | 255 | 6826 | 1028 | | | | | 19796 | 27782 | |
| 47 | CRN79674.1 | 259 | 6830 | 1028 | | | | | 19796 | 27782 | |
| 48 | CRO17354.1 | 263 | 6834 | 1083 | | | | | 7536 | 15377 | |
| 49 | CRW80513.1 | 267 | 6838 | 1028 | | | | | 1009 | 8995 | |
| 50 | CRW81768.1 | 271 | 6842 | 1083 | | | | | 3068 | 10909 | |
| 51 | CRS64331.1 | 275 | 6846 | 1083 | | | | | 7536 | 15377 | |
| 52 | CRS61286.1 | 279 | 6850 | 1028 | | | | | 21981 | 29967 | |
| 53 | CRX10264.1 | 283 | 6854 | 1083 | | | | | 7536 | 15377 | |
| 54 | CRX00531.1 | 287 | 6858 | 1028 | | | | | 21972 | 29958 | |
| 55 | CRW55874.1 | 291 | 6862 | 1083 | | | | | 7536 | 15377 | |
| 56 | CRX16408.1 | 295 | 6866 | 1028 | | | | | 10272 | 18258 | |
| 57 | CRP10908.1 | 299 | 6870 | 1028 | | | | | 19796 | 27782 | |
| 58 | CRP41378.1 | 303 | 6874 | 1083 | | | | | 20600 | 28441 | |
| 59 | CRP14671.1 | 307 | 6878 | 1028 | | | | | 21981 | 29967 | |
| 60 | CRP47678.1 | 311 | 6882 | 1083 | | | | | 7536 | 15377 | |
| 61 | CRO88182.1 | 315 | 6886 | 1083 | | | | | 7536 | 15377 | |
| 62 | CRO42689.1 | 319 | 6890 | 1028 | | | | | 21982 | 29968 | |
| 63 | CRN87527.1 | 323 | 6894 | 1083 | | | | | 7536 | 15377 | |
| 64 | CRN66270.1 | 327 | 6898 | 1028 | | | | | 19796 | 27782 | |
| 65 | CRP41135.1 | 331 | 6902 | 1028 | | | | | 19796 | 27782 | |
| 66 | CRP68434.1 | 335 | 6906 | 1083 | | | | | 7536 | 15377 | |
| 67 | CRP76218.1 | 339 | 6910 | 1028 | | | | | 21981 | 29967 | |
| 68 | CRQ24486.1 | 343 | 6914 | 1083 | | | | | 7536 | 15377 | |
| 69 | CRP82303.1 | 347 | 6918 | 1083 | | | | | 7536 | 15377 | |
| 70 | CRP60955.1 | 351 | 6922 | 1028 | | | | | 21979 | 29965 | |
| 71 | CRP29619.1 | 355 | 6926 | 1083 | | | | | 7536 | 15377 | |
| 72 | CRO50908.1 | 359 | 6930 | 1028 | | | | | 19796 | 27782 | |
| 73 | | | | | | | | | 2197 | 6787 | |
| 74 | CRP88395.1 | 366 | 6937 | 1028 | | | | | 15384 | 23370 | |
| 75 | CRP42036.1 | 370 | 6941 | 1028 | | | | | 21981 | 29967 | |
| 76 | CRP91116.1 | 374 | 6945 | 1083 | | | | | 7536 | 15377 | |
| 77 | CRN61529.1 | 378 | 6949 | 1028 | | | | | 21982 | 29968 | |
| 78 | CRN91506.1 | 382 | 6953 | 1083 | | | | | 20602 | 28443 | |
| 79 | CRP44801.1 | 386 | 6957 | 1083 | | | | | 20602 | 28443 | |
| 80 | CRP16211.1 | 390 | 6961 | 1028 | | | | | 21981 | 29967 | |
| 81 | CRP57132.1 | 394 | 6965 | 1028 | | | | | 19796 | 27782 | |
| 82 | CRP83812.1 | 398 | 6969 | 1083 | | | | | 20602 | 28443 | |
| 83 | CRO13657.1 | 402 | 6973 | 1028 | | | | | 28606 | 36592 | |
| 84 | CRO55518.1 | 406 | 6977 | 1083 | | | | | 7536 | 15377 | |
| 85 | CRO41650.1 | 410 | 6981 | 1083 | | | | | 20602 | 28443 | |
| 86 | CRO12755.1 | 414 | 6985 | 1028 | | | | | 19796 | 27782 | |
| 87 | CRP86290.1 | 418 | 6989 | 1083 | | | | | 20602 | 28443 | |
| 88 | CRP43574.1 | 422 | 6993 | 1028 | | | | | 21972 | 29958 | |
| 89 | CRP83305.1 | 426 | 6997 | 1083 | | | | | 7536 | 15377 | |
| 90 | CRP15159.1 | 430 | 7001 | 1028 | | | | | 19797 | 27783 | |
| 91 | CRP60560.1 | 434 | 7005 | 1028 | | | | | 21977 | 29963 | |
| 92 | CRQ10764.1 | 438 | 7009 | 1083 | | | | | 7536 | 15377 | |
| 93 | CRP54752.1 | 442 | 7013 | 1083 | | | | | 7536 | 15377 | |
| 94 | CRP76522.1 | 446 | 7017 | 1028 | | | | | 19796 | 27782 | |
| 95 | CRP46003.1 | 450 | 7021 | 1083 | | | | | 20602 | 28443 | |
| 96 | CRP23116.1 | 454 | 7025 | 1028 | | | | | 21977 | 29963 | |
| 97 | CRP65472.1 | 458 | 7029 | 1028 | | | | | 19796 | 27782 | |
| 98 | CRP93795.1 | 462 | 7033 | 1083 | | | | | 7536 | 15377 | |
| 99 | CRQ27545.1 | 466 | 7037 | 1028 | | | | | 19797 | 27783 | |
| 100 | CRQ56549.1 | 470 | 7041 | 1083 | | | | | 7536 | 15377 | |
| 101 | CRP80501.1 | 474 | 7045 | 1083 | | | | | 20602 | 28443 | |
| 102 | CRP35990.1 | 478 | 7049 | 1028 | | | | | 21982 | 29968 | |
| 103 | CRO87201.1 | 482 | 7053 | 1028 | | | | | 19796 | 27782 | |
| 104 | CRP11419.1 | 486 | 7057 | 1083 | | | | | 7536 | 15377 | |
| 105 | CRP24877.1 | 490 | 7061 | 1083 | | | | | 7536 | 15377 | |
| 106 | CRO90725.1 | 494 | 7065 | 1028 | | | | | 19796 | 27782 | |
| 107 | CRP17797.1 | 498 | 7069 | 1083 | | | | | 7536 | 15377 | |
| 108 | CRO58922.1 | 502 | 7073 | 1028 | | | | | 19796 | 27782 | |
| 109 | CRP26962.1 | 506 | 7077 | 1028 | | | | | 21977 | 29963 | |
| 110 | CRP96154.1 | 510 | 7081 | 1083 | | | | | 5235 | 13076 | |
| 111 | CRN60218.1 | 514 | 7085 | 1028 | | | | | 19796 | 27782 | |
| 112 | CRO00192.1 | 518 | 7089 | 1083 | | | | | 7536 | 15377 | |
| 113 | CRO92517.1 | 522 | 7093 | 1083 | | | | | 7536 | 15377 | |
| 114 | CRO70260.1 | 526 | 7097 | 1028 | | | | | 19796 | 27782 | |
| 115 | CRP22828.1 | 530 | 7101 | 1028 | | | | | 21981 | 29967 | |
| 116 | CRP50508.1 | 534 | 7105 | 1083 | | | | | 7536 | 15377 | |
| 117 | CRO39287.1 | 538 | 7109 | 1083 | | | | | 20601 | 28442 | |
| 118 | CRO06853.1 | 542 | 7113 | 1028 | | | | | 19796 | 27782 | |
| 119 | CRP42088.1 | 546 | 7117 | 1083 | | | | | 20602 | 28443 | |
| 120 | CRP08999.1 | 550 | 7121 | 1028 | | | | | 21977 | 29963 | |
| 121 | CRQ56997.1 | 554 | 7125 | 1083 | | | | | 7536 | 15377 | |
| 122 | CRQ59380.1 | 558 | 7129 | 1028 | | | | | 19796 | 27782 | |
| 123 | CRQ91375.1 | 562 | 7133 | 1083 | | | | | 7536 | 15377 | |
| 124 | CRQ84097.1 | 566 | 7137 | 1028 | | | | | 21977 | 29963 | |
| 125 | CRQ52244.1 | 570 | 7141 | 1083 | | | | | 7536 | 15377 | |
| 126 | CRQ54306.1 | 574 | 7145 | 1028 | | | | | 19796 | 27782 | |
| 127 | CRQ62236.1 | 578 | 7149 | 1083 | | | | | 7536 | 15377 | |
| 128 | CRQ81684.1 | 582 | 7153 | 1028 | | | | | 21977 | 29963 | |
| 129 | CRQ17181.1 | 586 | 7157 | 1028 | | | | | 19796 | 27782 | |
| 130 | CRQ37577.1 | 589 | 7160 | 1083 | | | | | 20602 | 28443 | |
| 131 | CRQ86033.1 | 593 | 7164 | 1083 | | | | | 3165 | 11006 | |
| 132 | CRQ40649.1 | 597 | 7168 | 1028 | | | | | 21981 | 29967 | |
| 133 | CRQ91563.1 | 601 | 7172 | 1083 | | | | | 20602 | 28443 | |
| 134 | CRQ79048.1 | 605 | 7176 | 1028 | | | | | 21981 | 29967 | |
| 135 | CRR29633.1 | 609 | 7180 | 1083 | | | | | 7536 | 15377 | |
| 136 | CRR29050.1 | 613 | 7184 | 1028 | | | | | 10273 | 18259 | |
| 137 | CRR72814.1 | 617 | 7188 | 1028 | | | | | 19796 | 27782 | |
| 138 | CRR80212.1 | 621 | 7192 | 1083 | | | | | 7536 | 15377 | |
| 139 | CRR16565.1 | 625 | 7196 | 1083 | | | | | 20602 | 28443 | |
| 140 | CRR21548.1 | 629 | 7200 | 1028 | | | | | 21979 | 29965 | |
| 141 | CRQ92439.1 | 633 | 7204 | 1083 | | | | | 7536 | 15377 | |
| 142 | CRQ86092.1 | 637 | 7208 | 1028 | | | | | 19796 | 27782 | |
| 143 | CRQ59643.1 | 641 | 7212 | 1083 | | | | | 20602 | 28443 | |
| 144 | CRQ56267.1 | 645 | 7216 | 1028 | | | | | 19796 | 27782 | |
| 145 | CRR63880.1 | 649 | 7220 | 1083 | | | | | 20602 | 28443 | |
| 146 | CRR21712.1 | 653 | 7224 | 1028 | | | | | 21977 | 29963 | |
| 147 | | | | | | | | | 2348 | 6938 | |
| 148 | CRP93286.1 | 660 | 7231 | 1028 | | | | | 15000 | 22986 | |
| 149 | CRR90653.1 | 664 | 7235 | 1028 | | | | | 19796 | 27782 | |
| 150 | CRS00769.1 | 668 | 7239 | 1083 | | | | | 20650 | 28491 | |
| 151 | CRR57278.1 | 672 | 7243 | 1028 | | | | | 21981 | 29967 | |
| 152 | CRR67560.1 | 676 | 7247 | 1083 | | | | | 20600 | 28441 | |
| 153 | CRR87246.1 | 680 | 7251 | 1028 | | | | | 21981 | 29967 | |
| 154 | CRS01368.1 | 684 | 7255 | 1083 | | | | | 20650 | 28491 | |
| 155 | CRX14802.1 | 688 | 7259 | 1083 | | | | | 20571 | 28412 | |
| 156 | CRX13614.1 | 692 | 7263 | 1028 | | | | | 19796 | 27782 | |
| 157 | CRX26810.1 | 696 | 7267 | 1028 | | | | | 10207 | 18193 | |
| 158 | | | | | | | | | 20601 | 25191 | |
| 159 | CRP16606.1 | 703 | 7274 | 1028 | | | | | 21972 | 29958 | |
| 160 | CRP62133.1 | 707 | 7278 | 1083 | | | | | 7536 | 15377 | |
| 161 | CRO03492.1 | 711 | 7282 | 1028 | | | | | 21974 | 29960 | |
| 162 | CRO33372.1 | 715 | 7286 | 1083 | | | | | 7536 | 15377 | |
| 163 | CRS11153.1 | 719 | 7290 | 1028 | | | | | 21977 | 29963 | |
| 164 | CRS17556.1 | 723 | 7294 | 1083 | | | | | 7536 | 15377 | |
| 165 | CRR97375.1 | 727 | 7298 | 1028 | | | | | 19796 | 27782 | |
| 166 | CRS12921.1 | 731 | 7302 | 1083 | | | | | 20650 | 28491 | |
| 167 | CRP70784.1 | 735 | 7306 | 1028 | | | | | 19796 | 27782 | |
| 168 | CRO21080.1 | 739 | 7310 | 1083 | | | | | 7536 | 15377 | |
| 169 | CRO99381.1 | 743 | 7314 | 1028 | | | | | 19796 | 27782 | |
| 170 | CRP25040.1 | 747 | 7318 | 1083 | | | | | 7536 | 15377 | |
| 171 | CRP38642.1 | 751 | 7322 | 1083 | | | | | 7536 | 15377 | |
| 172 | CRP11550.1 | 755 | 7326 | 1028 | | | | | 21982 | 29968 | |
| 173 | CRP98584.1 | 759 | 7330 | 1083 | | | | | 20650 | 28491 | |
| 174 | CRP72862.1 | 763 | 7334 | 1028 | | | | | 21977 | 29963 | |
| 175 | CRX06581.1 | 767 | 7338 | 1028 | | | | | 19796 | 27782 | |
| 176 | CRX13436.1 | 771 | 7342 | 1083 | | | | | 20650 | 28491 | |
| 177 | CRR85682.1 | 775 | 7346 | 1028 | | | | | 21981 | 29967 | |
| 178 | CRR95301.1 | 779 | 7350 | 1083 | | | | | 7536 | 15377 | |
| 179 | CRR86731.1 | 783 | 7354 | 1083 | | | | | 7536 | 15377 | |
| 180 | CRR89302.1 | 787 | 7358 | 1028 | | | | | 19796 | 27782 | |
| 181 | CRS01767.1 | 791 | 7362 | 1028 | | | | | 19796 | 27782 | |
| 182 | CRS11319.1 | 795 | 7366 | 1083 | | | | | 7536 | 15377 | |
| 183 | CRR80885.1 | 799 | 7370 | 1028 | | | | | 21985 | 29971 | |
| 184 | CRR92362.1 | 803 | 7374 | 1083 | | | | | 7536 | 15377 | |
| 185 | CRR30192.1 | 807 | 7378 | 1083 | | | | | 7536 | 15377 | |
| 186 | CRS13888.1 | 811 | 7382 | 1028 | | | | | 19797 | 27783 | |
| 187 | CRS13748.1 | 815 | 7386 | 1083 | | | | | 7536 | 15377 | |
| 188 | CRR98713.1 | 819 | 7390 | 1028 | | | | | 19796 | 27782 | |
| 189 | CRS06172.1 | 823 | 7394 | 1028 | | | | | 21977 | 29963 | |
| 190 | CRS13985.1 | 827 | 7398 | 1083 | | | | | 20602 | 28443 | |
| 191 | CRW73996.1 | 831 | 7402 | 617 | | | | | 9779 | 16532 | |
| 192 | CRX24341.1 | 835 | 7406 | 1083 | | | | | 20602 | 28443 | |
| 193 | CRX24406.1 | 839 | 7410 | 1083 | | | | | 7536 | 15377 | |
| 194 | CRX17822.1 | 843 | 7414 | 1028 | | | | | 15348 | 23334 | |
| 195 | CRR62878.1 | 847 | 7418 | 1083 | | | | | 7536 | 15377 | |
| 196 | CRX06818.1 | 851 | 7422 | 1028 | | | | | 21984 | 29970 | |
| 197 | CRX13750.1 | 855 | 7426 | 1083 | | | | | 20602 | 28443 | |
| 198 | CRR17545.1 | 859 | 7430 | 1083 | | | | | 6790 | 14631 | |
| 199 | CRQ30075.1 | 863 | 7434 | 1028 | | | | | 10239 | 18225 | |
| 200 | CRR51884.1 | 867 | 7438 | 1083 | | | | | 7536 | 15377 | |
| 201 | CRQ97126.1 | 871 | 7442 | 1028 | | | | | 21982 | 29968 | |
| 202 | KQJ58849.1 | 875 | 7446 | 1012 | | | | | 96 | 8034 | |
| 203 | KRU98871.1 | 879 | 7450 | 1083 | | | | | 4551 | 12425 | |
| 204 | KRV03413.1 | 883 | 7454 | 1083 | | | | | 10551 | 18423 | |
| 205 | KRU83197.1 | 887 | 7458 | 1083 | | | | | 3589 | 11464 | |
| 206 | KRU92654.1 | 891 | 7462 | 1083 | | | | | 4551 | 12425 | |
| 207 | KSD25192.1 | 895 | 7466 | 1013 | | | | | 113233 | 121174 | |
| 208 | KSD74105.1 | 899 | 7470 | 1013 | | | | | 106671 | 114764 | |
| 209 | KSD60221.1 | 903 | 7474 | 1028 | | | | | 115376 | 123362 | |
| 210 | KSD87955.1 | 907 | 7478 | 1083 | | | | | 7720 | 15561 | |
| 211 | KSD88702.1 | 911 | 7482 | 1028 | | | | | 20150 | 28136 | |
| 212 | KSD50143.1 | 915 | 7486 | 1083 | | | | | 7720 | 15561 | |
| 213 | KSD56742.1 | 919 | 7490 | 1028 | | | | | 202494 | 210480 | |
| 214 | KSE06416.1 | 923 | 7494 | 1028 | | | | | 691615 | 699601 | |
| 215 | KSD97267.1 | 927 | 7498 | 1083 | | | | | 8459 | 16300 | |
| 216 | KSE55323.1 | 931 | 7502 | 1083 | | | | | 7720 | 15561 | |
| 217 | KSE57197.1 | 935 | 7506 | 1028 | | | | | 20032 | 28018 | |
| 218 | KSE67394.1 | 939 | 7510 | 1083 | | | | | 7720 | 15561 | |
| 219 | KSE22577.1 | 943 | 7514 | 1083 | | | | | 7720 | 15561 | |
| 220 | KSE22792.1 | 947 | 7518 | 1028 | | | | | 19980 | 27966 | |
| 221 | KSG55973.1 | 951 | 7522 | 1013 | | | | | 113233 | 121174 | |
| 222 | KSH11348.1 | 955 | 7526 | 1013 | | | | | 114765 | 122706 | |
| 223 | KSC39035.1 | 959 | 7530 | 1083 | | | | | 3729 | 11602 | |
| 224 | KSC26156.1 | 963 | 7534 | 1083 | | | | | 607382 | 615223 | |
| 225 | KSC92705.1 | 967 | 7538 | 1013 | | | | | 113233 | 121174 | |
| 226 | KSD59504.1 | 971 | 7542 | 1028 | | | | | 20152 | 28138 | |
| 227 | KSD58173.1 | 975 | 7546 | 1083 | | | | | 7720 | 15561 | |
| 228 | KSE83339.1 | 979 | 7550 | 1013 | | | | | 111993 | 119934 | |
| 229 | KSI47362.1 | 983 | 7554 | 1083 | | | | | 26399 | 34240 | |
| 230 | KSI07555.1 | 987 | 7558 | 1083 | | | | | 7720 | 15561 | |
| 231 | KSJ10821.1 | 991 | 7562 | 1028 | | | | | 28779 | 36765 | |
| 232 | KSJ26232.1 | 995 | 7566 | 1013 | | | | | 280039 | 287980 | |
| 233 | KSJ98816.1 | 999 | 7570 | 1083 | | | | | 178760 | 186632 | |
| 234 | KSK91644.1 | 1003 | 7574 | 1083 | | | | | 44277 | 52118 | |
| 235 | KSL06913.1 | 1007 | 7578 | 1083 | | | | | 6696 | 14569 | |
| 236 | KSM47318.1 | 1011 | 7582 | 1083 | | | | | 186153 | 194025 | |
| 237 | KSM41522.1 | 1015 | 7586 | 1013 | | | | | 174276 | 182369 | |
| 238 | KSN32762.1 | 1019 | 7590 | 1083 | | | | | 20786 | 28627 | |
| 239 | KSN33323.1 | 1023 | 7594 | 1028 | | | | | 20152 | 28138 | |
| 240 | KSN27909.1 | 1027 | 7598 | 1083 | | | | | 5029 | 12870 | |
| 241 | KSN02520.1 | 1031 | 7602 | 1014 | | | | | 88424 | 96278 | |
| 242 | KSN94645.1 | 1035 | 7606 | 1083 | | | | | 6822 | 14695 | |
| 243 | KSN59776.1 | 1039 | 7610 | 1028 | | | | | 19980 | 27966 | |
| 244 | KSN68238.1 | 1043 | 7614 | 1083 | | | | | 678802 | 686643 | |
| 245 | KSO78874.1 | 1047 | 7618 | 1083 | | | | | 6821 | 14694 | |
| 246 | KSO73923.1 | 1051 | 7622 | 1083 | | | | | 7720 | 15561 | |
| 247 | KSO74311.1 | 1055 | 7626 | 1028 | | | | | 28768 | 36754 | |
| 248 | KSO93084.1 | 1059 | 7630 | 1028 | | | | | 19851 | 27837 | |
| 249 | KSO91842.1 | 1063 | 7634 | 1083 | | | | | 7720 | 15561 | |
| 250 | KSP60334.1 | 1067 | 7638 | 1028 | | | | | 19084 | 27070 | |
| 251 | KSP82593.1 | 1071 | 7642 | 1009 | | | | | 65229 | 73525 | |
| 252 | KSQ11853.1 | 1075 | 7646 | 1083 | | | | | 262790 | 270663 | |
| 253 | KSN68772.1 | 1079 | 7650 | 1083 | | | | | 6822 | 14695 | |
| 254 | KSO40827.1 | 1083 | 7654 | 1013 | | | | | 176313 | 184406 | |
| 255 | KSQ48397.1 | 1087 | 7658 | 1028 | | | | | 29036 | 37022 | |
| 256 | KSQ47081.1 | 1091 | 7662 | 1083 | | | | | 7720 | 15561 | |
| 257 | KSQ03905.1 | 1095 | 7666 | 1028 | | | | | 29276 | 37262 | |
| 258 | KSQ01965.1 | 1099 | 7670 | 1083 | | | | | 7720 | 15561 | |
| 259 | KSR61591.1 | 1103 | 7674 | 1013 | | | | | 101987 | 110080 | |
| 260 | KSR90899.1 | 1107 | 7678 | 1013 | | | | | 112375 | 120316 | |
| 261 | KSR87676.1 | 1111 | 7682 | 1083 | | | | | 6822 | 14695 | |
| 262 | KSR56103.1 | 1115 | 7686 | 1083 | | | | | 7720 | 15561 | |
| 263 | KSR68840.1 | 1119 | 7690 | 1028 | | | | | 691615 | 699601 | |
| 264 | KSS83645.1 | 1123 | 7694 | 1028 | | | | | 28814 | 36800 | |
| 265 | KSS82294.1 | 1127 | 7698 | 1083 | | | | | 7720 | 15561 | |
| 266 | KSS65849.1 | 1131 | 7702 | 1083 | | | | | 7720 | 15561 | |
| 267 | KSS71071.1 | 1135 | 7706 | 1028 | | | | | 65766 | 73752 | |
| 268 | KSS83668.1 | 1139 | 7710 | 1083 | | | | | 7720 | 15561 | |
| 269 | KSS97186.1 | 1143 | 7714 | 1028 | | | | | 691552 | 699538 | |
| 270 | KSS68539.1 | 1147 | 7718 | 1083 | | | | | 7720 | 15561 | |
| 271 | KSS64136.1 | 1151 | 7722 | 1028 | | | | | 303502 | 311488 | |
| 272 | KST02404.1 | 1155 | 7726 | 1028 | | | | | 20083 | 28069 | |
| 273 | KST01503.1 | 1159 | 7730 | 1083 | | | | | 7720 | 15561 | |
| 274 | KST09050.1 | 1163 | 7734 | 1028 | | | | | 28768 | 36754 | |
| 275 | KST07353.1 | 1167 | 7738 | 1083 | | | | | 7719 | 15560 | |
| 276 | KSR39891.1 | 1171 | 7742 | 1028 | | | | | 123377 | 131363 | |
| 277 | KST03846.1 | 1175 | 7746 | 1083 | | | | | 7720 | 15561 | |
| 278 | KST05248.1 | 1179 | 7750 | 1028 | | | | | 28768 | 36754 | |
| 279 | KSS85818.1 | 1183 | 7754 | 1028 | | | | | 20152 | 28138 | |
| 280 | KSS83935.1 | 1187 | 7758 | 1083 | | | | | 7720 | 15561 | |
| 281 | KTF54399.1 | 1191 | 7762 | 1028 | | | | | 20040 | 28026 | |
| 282 | KTF49808.1 | 1195 | 7766 | 1083 | | | | | 7726 | 15567 | |
| 283 | KTK55769.1 | 1199 | 7770 | 964 | | | | | 5885944 | 5897844 | |
| 284 | ALZ13981.1 | 1203 | 7774 | 1009 | | | | | 3432864 | 3441160 | |
| 285 | ALY33853.1 | 1207 | 7778 | 1082 | | | | | 398410 | 406279 | |
| 286 | ALY39463.1 | 1211 | 7782 | 1083 | | | | | 155293 | 163134 | |
| 287 | ALY43154.1 | 1215 | 7786 | 1013 | | | | | 4323402 | 4331343 | |
| 288 | | | | | | | | | 3032150 | 3036740 | |
| 289 | KWZ69448.1 | 1221 | 7792 | 1013 | | | | | 10132 | 18224 | |
| 290 | KXG16568.1 | 1225 | 7796 | 1013 | | | | | 153843 | 161784 | |
| 291 | KYO75363.1 | 1229 | 7800 | 1028 | | | | | 19761 | 27747 | |
| 292 | KIS48445.1 | 1232 | 7803 | 536 | | | | | 2364174 | 2369453 | |
| 293 | KVA56603.1 | 1236 | 7807 | 1198 | | | | | 199542 | 207683 | |
| 294 | KVC33768.1 | 1240 | 7811 | 860 | | | | | 118414 | 125547 | |
| 295 | AEV65171.1 | 1244 | 7815 | 1082 | | | | | 6013571 | 6021027 | |
| 296 | KGE64071.1 | 1248 | 7819 | 1082 | | | | | 75257 | 83079 | |
| 297 | KWV80503.1 | 1252 | 7823 | 1009 | | | | | 1368440 | 1376687 | |
| 298 | ABQ80735.1 | 1256 | 7827 | 1082 | | | | | 5169632 | 5177451 | |
| 299 | AFK68694.1 | 1260 | 7831 | 1083 | | | | | 1915596 | 1923415 | |
| 300 | EMR48533.1 | 1264 | 7835 | 1012 | | | | | 523291 | 531481 | |
| 301 | AGA73020.1 | 1268 | 7839 | 1095 | | | | | 2309435 | 2317296 | |
| 302 | KTC24574.1 | 1272 | 7843 | 1009 | | | | | 91397 | 99402 | |
| 303 | ABP79170.1 | 1276 | 7847 | 1012 | | | | | 1580205 | 1591257 | Additional ZorC homolog ABP79169.1 |
| 304 | AEA83400.1 | 1280 | 7851 | 1012 | | | | | 1567955 | 1576354 | |
| 305 | EHY79733.1 | 1284 | 7855 | 1012 | | | | | 65479 | 73878 | |
| 306 | AFM32809.1 | 1288 | 7859 | 1012 | | | | | 1676860 | 1685262 | |
| 307 | EWC39444.1 | 1292 | 7863 | 1083 | | | | | 336964 | 344689 | |
| 308 | EPL62185.1 | 1295 | 7866 | 1012 | | | | | 213132 | 221531 | |
| 309 | EPM44378.1 | 1299 | 7870 | 1082 | | | | | 14136 | 21754 | |
| 310 | EPN29006.1 | 1303 | 7874 | 177 | | | | | 667 | 5568 | |
| 311 | EPN47281.1 | 1307 | 7878 | 183 | | | | | 2221 | 7140 | |
| 312 | EPN38586.1 | 1311 | 7882 | 184 | | | | | 2215 | 7137 | |
| 313 | EPN31619.1 | 1315 | 7886 | 928 | | | | | 16485 | 30385 | longer system - ZorD annotated manually |
| 314 | EPN22778.1 | 1319 | 7890 | 1082 | | | | | 16485 | 24103 | |
| 315 | EPN13935.1 | 1323 | 7894 | 1082 | | | | | 1746 | 9364 | |
| 316 | EPM64845.1 | 1327 | 7898 | 1082 | | | | | 677 | 8295 | |
| 317 | KFE44520.1 | 1331 | 7902 | 1082 | | | | | 131424 | 139246 | |
| 318 | KTC49635.1 | 1335 | 7906 | 1082 | | | | | 2323 | 10154 | |
| 319 | AAM40750.1 | 1339 | 7910 | 1065 | | | | | 1688713 | 1696136 | |
| 320 | AAY49834.1 | 1343 | 7914 | 1065 | | | | | 3347629 | 3355052 | |
| 321 | AKS18352.1 | 1347 | 7918 | 641 | | | | | 3124168 | 3131591 | |
| 322 | ACP24375.1 | 1351 | 7922 | 1072 | | | | | 631821 | 638722 | |
| 323 | | | | | | | | | 41480 | 45803 | |
| 324 | KUM27285.1 | 1358 | 7929 | 970 | | | | | 191211 | 198647 | |
| 325 | CDZ72585.1 | 1362 | 7933 | 1033 | | | | | 110001 | 117427 | |
| 326 | ACK84142.1 | 1366 | 7937 | 1033 | | | | | 3482023 | 3489384 | |
| 327 | CAH15305.1 | 1370 | 7941 | 1128 | | | | | 1195308 | 1202768 | |
| 328 | CCD05129.1 | 1374 | 7945 | 1128 | | | | | 1099307 | 1106704 | |
| 329 | CZL99023.1 | 1378 | 7949 | 1128 | | | | | 8467 | 15864 | |
| 330 | CZN28496.1 | 1382 | 7953 | 1128 | | | | | 8467 | 15864 | |
| 331 | CZP40238.1 | 1386 | 7957 | 1128 | | | | | 8467 | 15864 | |
| 332 | CZP31232.1 | 1390 | 7961 | 1128 | | | | | 37176 | 44573 | |
| 333 | CZP19776.1 | 1394 | 7965 | 1128 | | | | | 8467 | 15864 | |
| 334 | CZN56551.1 | 1398 | 7969 | 1128 | | | | | 8467 | 15864 | |
| 335 | CZN21905.1 | 1402 | 7973 | 1128 | | | | | 8467 | 15864 | |
| 336 | CZP49331.1 | 1406 | 7977 | 1128 | | | | | 8467 | 15864 | |
| 337 | CZP61158.1 | 1410 | 7981 | 1128 | | | | | 37472 | 44869 | |
| 338 | CZN58367.1 | 1414 | 7985 | 1128 | | | | | 37472 | 44869 | |
| 339 | CZO62490.1 | 1418 | 7989 | 1128 | | | | | 37166 | 44563 | |
| 340 | CZO85517.1 | 1422 | 7993 | 1128 | | | | | 37472 | 44869 | |
| 341 | CZN18828.1 | 1426 | 7997 | 1128 | | | | | 37183 | 44580 | |
| 342 | CZO33299.1 | 1430 | 8001 | 1128 | | | | | 37166 | 44563 | |
| 343 | CZN48822.1 | 1434 | 8005 | 1128 | | | | | 148086 | 155483 | |
| 344 | CZO35416.1 | 1438 | 8009 | 1128 | | | | | 37456 | 44853 | |
| 345 | CZL93564.1 | 1442 | 8013 | 1128 | | | | | 131968 | 139365 | |
| 346 | CZH31874.1 | 1446 | 8017 | 1128 | | | | | 8467 | 15864 | |
| 347 | CZG09164.1 | 1450 | 8021 | 1129 | | | | | 167538 | 174938 | |
| 348 | CZO58072.1 | 1454 | 8025 | 1128 | | | | | 8467 | 15864 | |
| 349 | CZP31158.1 | 1458 | 8029 | 1128 | | | | | 677 | 8074 | |
| 350 | CZP25160.1 | 1462 | 8033 | 1128 | | | | | 37183 | 44580 | |
| 351 | CZO87108.1 | 1466 | 8037 | 1128 | | | | | 37166 | 44563 | |
| 352 | CZP12686.1 | 1470 | 8041 | 1128 | | | | | 37456 | 44853 | |
| 353 | CZO76680.1 | 1474 | 8045 | 1128 | | | | | 8438 | 15835 | |
| 354 | CZM75981.1 | 1478 | 8049 | 1128 | | | | | 8467 | 15864 | |
| 355 | CZM80502.1 | 1482 | 8053 | 1128 | | | | | 37456 | 44853 | |
| 356 | CZO03187.1 | 1486 | 8057 | 1128 | | | | | 37166 | 44563 | |
| 357 | CZO99623.1 | 1490 | 8061 | 1128 | | | | | 8467 | 15864 | |
| 358 | CZN77929.1 | 1494 | 8065 | 1128 | | | | | 8467 | 15864 | |
| 359 | CZO69927.1 | 1498 | 8069 | 1128 | | | | | 37456 | 44853 | |
| 360 | CZO98759.1 | 1502 | 8073 | 1128 | | | | | 37472 | 44869 | |
| 361 | CZN87263.1 | 1506 | 8077 | 1128 | | | | | 37472 | 44869 | |
| 362 | CZO28947.1 | 1510 | 8081 | 1128 | | | | | 8467 | 15864 | |
| 363 | CZO58257.1 | 1514 | 8085 | 1128 | | | | | 37166 | 44563 | |
| 364 | CZM99444.1 | 1518 | 8089 | 1128 | | | | | 8467 | 15864 | |
| 365 | CZN97615.1 | 1522 | 8093 | 1128 | | | | | 37183 | 44580 | |
| 366 | CZN56267.1 | 1526 | 8097 | 1128 | | | | | 148086 | 155483 | |
| 367 | CZP07331.1 | 1530 | 8101 | 1128 | | | | | 8467 | 15864 | |
| 368 | CZP63613.1 | 1534 | 8105 | 1128 | | | | | 37440 | 44837 | |
| 369 | CZM58531.1 | 1538 | 8109 | 1128 | | | | | 8490 | 15887 | |
| 370 | CZN95629.1 | 1542 | 8113 | 1128 | | | | | 8441 | 15838 | |
| 371 | CZN53220.1 | 1546 | 8117 | 1128 | | | | | 37166 | 44563 | |
| 372 | CZN21830.1 | 1550 | 8121 | 1128 | | | | | 37472 | 44869 | |
| 373 | CZO53942.1 | 1554 | 8125 | 1128 | | | | | 8467 | 15864 | |
| 374 | CZO83111.1 | 1558 | 8129 | 1128 | | | | | 37472 | 44869 | |
| 375 | CZM80568.1 | 1562 | 8133 | 1128 | | | | | 8467 | 15864 | |
| 376 | CZN76211.1 | 1566 | 8137 | 1128 | | | | | 8467 | 15864 | |
| 377 | CZN17285.1 | 1570 | 8141 | 1128 | | | | | 37472 | 44869 | |
| 378 | CZN08698.1 | 1574 | 8145 | 1128 | | | | | 8472 | 15869 | |
| 379 | CZN07686.1 | 1578 | 8149 | 1128 | | | | | 8467 | 15864 | |
| 380 | CZN27100.1 | 1582 | 8153 | 1128 | | | | | 37456 | 44853 | |
| 381 | CZO91499.1 | 1586 | 8157 | 1128 | | | | | 8467 | 15864 | |
| 382 | CZN16051.1 | 1590 | 8161 | 1128 | | | | | 8467 | 15864 | |
| 383 | CZN38967.1 | 1594 | 8165 | 1128 | | | | | 8467 | 15864 | |
| 384 | CZI18617.1 | 1598 | 8169 | 1128 | | | | | 8467 | 15864 | |
| 385 | CZL86257.1 | 1602 | 8173 | 1128 | | | | | 8467 | 15864 | |
| 386 | CZM41687.1 | 1606 | 8177 | 1128 | | | | | 37166 | 44563 | |
| 387 | CZM78452.1 | 1610 | 8181 | 1128 | | | | | 8467 | 15864 | |
| 388 | CZM56718.1 | 1614 | 8185 | 1128 | | | | | 8467 | 15864 | |
| 389 | CZM62068.1 | 1618 | 8189 | 1128 | | | | | 37472 | 44869 | |
| 390 | CZM06036.1 | 1622 | 8193 | 1128 | | | | | 8467 | 15864 | |
| 391 | CZM98015.1 | 1626 | 8197 | 1128 | | | | | 37456 | 44853 | |
| 392 | CZN32097.1 | 1630 | 8201 | 1128 | | | | | 37183 | 44580 | |
| 393 | CZN27816.1 | 1634 | 8205 | 1128 | | | | | 37183 | 44580 | |
| 394 | CZM96679.1 | 1638 | 8209 | 1128 | | | | | 8467 | 15864 | |
| 395 | CZN17375.1 | 1642 | 8213 | 1128 | | | | | 37166 | 44563 | |
| 396 | CZN17807.1 | 1646 | 8217 | 1128 | | | | | 8467 | 15864 | |
| 397 | CZN51221.1 | 1650 | 8221 | 1128 | | | | | 8467 | 15864 | |
| 398 | CZN80042.1 | 1654 | 8225 | 1128 | | | | | 8467 | 15864 | |
| 399 | CZN83589.1 | 1658 | 8229 | 1128 | | | | | 37183 | 44580 | |
| 400 | CZN87872.1 | 1662 | 8233 | 1128 | | | | | 8408 | 15805 | |
| 401 | CZN94066.1 | 1666 | 8237 | 1128 | | | | | 37183 | 44580 | |
| 402 | CZO07198.1 | 1670 | 8241 | 1128 | | | | | 37510 | 44907 | |
| 403 | CZO11915.1 | 1674 | 8245 | 1128 | | | | | 8467 | 15864 | |
| 404 | CZO09323.1 | 1678 | 8249 | 1128 | | | | | 8467 | 15864 | |
| 405 | CZN87980.1 | 1682 | 8253 | 1128 | | | | | 8500 | 15897 | |
| 406 | CZO32215.1 | 1686 | 8257 | 1128 | | | | | 37183 | 44580 | |
| 407 | CZO07237.1 | 1690 | 8261 | 1128 | | | | | 8467 | 15864 | |
| 408 | CZO04106.1 | 1694 | 8265 | 1128 | | | | | 8472 | 15869 | |
| 409 | CZN90727.1 | 1698 | 8269 | 1128 | | | | | 37183 | 44580 | |
| 410 | CZP18336.1 | 1702 | 8273 | 1128 | | | | | 8467 | 15864 | |
| 411 | CZP26215.1 | 1706 | 8277 | 1128 | | | | | 8467 | 15864 | |
| 412 | CZP25407.1 | 1710 | 8281 | 1128 | | | | | 8467 | 15864 | |
| 413 | CZN32022.1 | 1714 | 8285 | 1128 | | | | | 8467 | 15864 | |
| 414 | CZO49291.1 | 1718 | 8289 | 1128 | | | | | 37183 | 44580 | |
| 415 | CZO32798.1 | 1722 | 8293 | 1128 | | | | | 8467 | 15864 | |
| 416 | CZO62433.1 | 1726 | 8297 | 1128 | | | | | 37166 | 44563 | |
| 417 | CZO58360.1 | 1730 | 8301 | 1128 | | | | | 37456 | 44853 | |
| 418 | CZO85770.1 | 1734 | 8305 | 1128 | | | | | 8467 | 15864 | |
| 419 | CZP51187.1 | 1738 | 8309 | 1128 | | | | | 37440 | 44837 | |
| 420 | CZO57236.1 | 1742 | 8313 | 1128 | | | | | 148086 | 155483 | |
| 421 | CZO45203.1 | 1746 | 8317 | 1128 | | | | | 8467 | 15864 | |
| 422 | CZO91301.1 | 1750 | 8321 | 1128 | | | | | 37472 | 44869 | |
| 423 | CZN53915.1 | 1754 | 8325 | 1128 | | | | | 37183 | 44580 | |
| 424 | CZN51502.1 | 1758 | 8329 | 1128 | | | | | 37440 | 44837 | |
| 425 | CZO76121.1 | 1762 | 8333 | 1128 | | | | | 37183 | 44580 | |
| 426 | CZR30106.1 | 1766 | 8337 | 1128 | | | | | 435 | 7832 | |
| 427 | KTD18863.1 | 1770 | 8341 | 1125 | | | | | 828 | 8210 | |
| 428 | | | | | | | | | 5615 | 9801 | |
| 429 | | | | | | | | | 2638 | 6824 | |
| 430 | EMT94536.1 | 1780 | 8351 | 1067 | | | | | 7878 | 15300 | |
| 431 | | | | | | | | | 1 | 3978 | |
| 432 | EMU02179.1 | 1787 | 8358 | 1067 | | | | | 2863 | 10285 | |
| 433 | EXB30085.1 | 1791 | 8362 | 1067 | | | | | 38504 | 45935 | |
| 434 | EXB35596.1 | 1795 | 8366 | 1067 | | | | | 190269 | 197700 | |
| 435 | EXB05629.1 | 1799 | 8370 | 1067 | | | | | 29426 | 36857 | |
| 436 | EXB92792.1 | 1803 | 8374 | 1067 | | | | | 600238 | 607669 | |
| 437 | EXB73474.1 | 1807 | 8378 | 1067 | | | | | 187080 | 194511 | |
| 438 | EXE59931.1 | 1811 | 8382 | 1067 | | | | | 198540 | 205983 | |
| 439 | EXE71115.1 | 1815 | 8386 | 717 | | | | | 5498 | 11879 | |
| 440 | KCX96447.1 | 1819 | 8390 | 1067 | | | | | 1709 | 9140 | |
| 441 | KHO17161.1 | 1823 | 8394 | 1067 | | | | | 15406 | 22828 | |
| 442 | KLT90614.1 | 1827 | 8398 | 1067 | | | | | 4629 | 12051 | |
| 443 | KRI43457.1 | 1831 | 8402 | 1068 | | | | | 19553 | 26974 | |
| 444 | KRJ35149.1 | 1835 | 8406 | 1067 | | | | | 9974 | 17405 | |
| 445 | AMN00866.1 | 1839 | 8410 | 1070 | | | | | 1372997 | 1380433 | |
| 446 | KDB58589.1 | 1843 | 8414 | 1300 | | | | | 192445 | 200726 | |
| 447 | KDD86367.1 | 1847 | 8418 | 1293 | | | | | 46614 | 54916 | |
| 448 | ACV76376.1 | 1851 | 8422 | 1034 | | | | | 19297 | 26557 | |
| 449 | AEI38593.1 | 1855 | 8426 | 1034 | | | | | 2402 | 9662 | |
| 450 | KTJ39357.1 | 1859 | 8430 | 1081 | | | | | 145121 | 152985 | |
| 451 | KUQ09079.1 | 1863 | 8434 | 1079 | | | | | 68203 | 76536 | |
| 452 | KYN64183.1 | 1867 | 8438 | 1042 | | | | | 702125 | 709468 | |
| 453 | AFP68827.1 | 1871 | 8442 | 1080 | | | | | 1024953 | 1032814 | Additional ZorC homolog AFP68828.1 |
| 454 | ESS59418.1 | 1875 | 8446 | 1080 | | | | | 17627 | 25491 | |
| 455 | EUL88644.1 | 1879 | 8450 | 78 | | | | | 3297579 | 3302381 | |
| 456 | KDF77541.1 | 1883 | 8454 | 78 | | | | | 65448 | 70250 | |
| 457 | AIX56043.1 | 1887 | 8458 | 1080 | | | | | 3771498 | 3779361 | |
| 458 | KJW77577.1 | 1891 | 8462 | 1079 | | | | | 1673 | 10006 | |
| 459 | KJX05613.1 | 1895 | 8466 | 1079 | | | | | 1673 | 10006 | |
| 460 | KPS87961.1 | 1898 | 8469 | 1035 | | | | | 1 | 5522 | |
| 461 | KRS24834.1 | 1901 | 8472 | 1080 | | | | | 6925 | 12583 | |
| 462 | KUQ03192.1 | 1905 | 8476 | 1080 | | | | | 42478 | 50340 | |
| 463 | BAK13747.1 | 1909 | 8480 | 1042 | | | | | 4393137 | 4400480 | |
| 464 | CCF11171.1 | 1913 | 8484 | 1042 | | | | | 3979011 | 3986353 | |
| 465 | AFR04656.1 | 1917 | 8488 | 1080 | | | | | 3684004 | 3691868 | |
| 466 | KHS69300.1 | 1921 | 8492 | 1080 | | | | | 46628 | 54492 | |
| 467 | KHT02548.1 | 1925 | 8496 | 1080 | | | | | 45831 | 53695 | |
| 468 | KHT03966.1 | 1929 | 8500 | 1080 | | | | | 47097 | 54961 | |
| 469 | ABV17208.1 | 1933 | 8504 | 1080 | | | | | 299412 | 307306 | |
| 470 | EIP59430.1 | 1937 | 8508 | 220 | | | | | 26726 | 32010 | |
| 471 | EIN79899.1 | 1941 | 8512 | 1080 | | | | | 3501 | 11365 | |
| 472 | EKK37355.1 | 1945 | 8516 | 1080 | | | | | 1607 | 9471 | |
| 473 | EKW33216.1 | 1949 | 8520 | 1080 | | | | | 2019 | 9883 | |
| 474 | ELW24561.1 | 1953 | 8524 | 1080 | | | | | 660 | 8524 | |
| 475 | ELV55441.1 | 1957 | 8528 | 1080 | | | | | 1478 | 9222 | |
| 476 | ELV58669.1 | 1961 | 8532 | 1080 | | | | | 664 | 8528 | |
| 477 | ELV27734.1 | 1965 | 8536 | 1080 | | | | | 64453 | 72317 | |
| 478 | ELE34847.1 | 1969 | 8540 | 1080 | | | | | 52842 | 60706 | |
| 479 | ELF60010.1 | 1973 | 8544 | 1084 | | | | | 586259 | 594135 | |
| 480 | ENC42749.1 | 1977 | 8548 | 1080 | | | | | 33688 | 41552 | |
| 481 | ENC49342.1 | 1981 | 8552 | 1080 | | | | | 27211 | 35075 | |
| 482 | ENC65369.1 | 1985 | 8556 | 1080 | | | | | 2255 | 9929 | |
| 483 | ENC60224.1 | 1989 | 8560 | 1080 | | | | | 27020 | 34884 | |
| 484 | ENC64691.1 | 1993 | 8564 | 1080 | | | | | 26939 | 34803 | |
| 485 | ENC77388.1 | 1997 | 8568 | 1080 | | | | | 26972 | 34836 | |
| 486 | ENC83661.1 | 2001 | 8572 | 1080 | | | | | 27153 | 35017 | |
| 487 | ENC86712.1 | 2005 | 8576 | 1080 | | | | | 27619 | 35483 | |
| 488 | ENE16671.1 | 2009 | 8580 | 1080 | | | | | 1801 | 9665 | |
| 489 | ENC77072.1 | 2013 | 8584 | 1080 | | | | | 27211 | 35075 | |
| 490 | EOQ56567.1 | 2017 | 8588 | 1079 | | | | | 636603 | 644930 | |
| 491 | ERB06044.1 | 2021 | 8592 | 1080 | | | | | 39898 | 47762 | |
| 492 | EQV94535.1 | 2025 | 8596 | 1079 | | | | | 962513 | 970840 | |
| 493 | EQV96724.1 | 2029 | 8600 | 1084 | | | | | 653799 | 661675 | |
| 494 | ERB23475.1 | 2033 | 8604 | 1080 | | | | | 1404484 | 1412348 | |
| 495 | ERA25536.1 | 2037 | 8608 | 1080 | | | | | 337494 | 345358 | |
| 496 | ERB97872.1 | 2041 | 8612 | 1080 | | | | | 2550 | 10414 | |
| 497 | ERB87463.1 | 2045 | 8616 | 1080 | | | | | 835 | 8699 | |
| 498 | ERB73279.1 | 2049 | 8620 | 1080 | | | | | 2197 | 10061 | |
| 499 | ERB84450.1 | 2053 | 8624 | 1080 | | | | | 5599 | 13463 | |
| 500 | ERD03125.1 | 2057 | 8628 | 1080 | | | | | 834 | 8698 | |
| 501 | ERD02931.1 | 2060 | 8631 | 111 | | | | | 2176 | 3966 | |
| 502 | ERD21115.1 | 2064 | 8635 | 1080 | | | | | 1073 | 8937 | |
| 503 | ERD19181.1 | 2068 | 8639 | 1080 | | | | | 2495 | 10359 | |
| 504 | ERD72946.1 | 2072 | 8643 | 1080 | | | | | 1149 | 9013 | |
| 505 | ERD66559.1 | 2076 | 8647 | 1080 | | | | | 600 | 8464 | |
| 506 | ERD75975.1 | 2080 | 8651 | 1080 | | | | | 945 | 8809 | |
| 507 | ERD83531.1 | 2084 | 8655 | 1080 | | | | | 658 | 8522 | |
| 508 | ERD92969.1 | 2088 | 8659 | 1080 | | | | | 1952 | 9816 | |
| 509 | ERD99053.1 | 2092 | 8663 | 1080 | | | | | 1388 | 9252 | |
| 510 | ERD98124.1 | 2096 | 8667 | 1080 | | | | | 1079 | 8943 | |
| 511 | AHM42042.1 | 2100 | 8671 | 1084 | | | | | 488627 | 496503 | |
| 512 | AHM46677.1 | 2104 | 8675 | 1084 | | | | | 496169 | 504045 | |
| 513 | AHM51232.1 | 2108 | 8679 | 1084 | | | | | 497566 | 505442 | |
| 514 | EYD87638.1 | 2112 | 8683 | 1079 | | | | | 41244 | 49112 | |
| 515 | EYW22453.1 | 2116 | 8687 | 1080 | | | | | 168471 | 176335 | |
| 516 | EYW31321.1 | 2120 | 8691 | 1080 | | | | | 1923 | 9787 | |
| 517 | EYV86552.1 | 2124 | 8695 | 1080 | | | | | 1922 | 9786 | |
| 518 | EYW71959.1 | 2128 | 8699 | 1080 | | | | | 26412 | 34276 | |
| 519 | EYW62760.1 | 2132 | 8703 | 1080 | | | | | 26412 | 34276 | |
| 520 | EYW60241.1 | 2136 | 8707 | 1080 | | | | | 1922 | 9786 | |
| 521 | EYW56541.1 | 2140 | 8711 | 1080 | | | | | 2143 | 10007 | |
| 522 | EYW47183.1 | 2144 | 8715 | 1080 | | | | | 3218 | 11082 | |
| 523 | EYW47503.1 | 2148 | 8719 | 1080 | | | | | 3222 | 11086 | |
| 524 | EYW43759.1 | 2152 | 8723 | 1080 | | | | | 26417 | 34281 | |
| 525 | EYW40417.1 | 2156 | 8727 | 1080 | | | | | 26412 | 34276 | |
| 526 | EYW30109.1 | 2160 | 8731 | 1080 | | | | | 1922 | 9786 | |
| 527 | EYY79854.1 | 2164 | 8735 | 1080 | | | | | 34684 | 42548 | |
| 528 | EZB10670.1 | 2168 | 8739 | 1080 | | | | | 2019 | 9883 | |
| 529 | EZE32943.1 | 2172 | 8743 | 1080 | | | | | 26396 | 34260 | |
| 530 | KDZ82664.1 | 2176 | 8747 | 1080 | | | | | 17465 | 25329 | |
| 531 | KHG75766.1 | 2180 | 8751 | 1080 | | | | | 581 | 8445 | |
| 532 | KHG79500.1 | 2184 | 8755 | 1080 | | | | | 921 | 8785 | |
| 533 | KOZ37429.1 | 2188 | 8759 | 1080 | | | | | 64411 | 72275 | |
| 534 | KOZ53463.1 | 2192 | 8763 | 1080 | | | | | 4236 | 12100 | |
| 535 | KOZ92706.1 | 2196 | 8767 | 1080 | | | | | 6079 | 13943 | |
| 536 | CTV73579.1 | 2200 | 8771 | 1080 | | | | | 14419 | 22283 | |
| 537 | CTY75874.1 | 2204 | 8775 | 1080 | | | | | 70490 | 78354 | |
| 538 | KPH47565.1 | 2208 | 8779 | 1080 | | | | | 596 | 8460 | |
| 539 | KPH29391.1 | 2212 | 8783 | 1080 | | | | | 26567 | 34431 | |
| 540 | KPO40057.1 | 2216 | 8787 | 1080 | | | | | 26789 | 34653 | |
| 541 | KPP40487.1 | 2220 | 8791 | 1080 | | | | | 27360 | 35224 | |
| 542 | KQI75361.1 | 2224 | 8795 | 1080 | | | | | 53820 | 61684 | |
| 543 | KST33202.1 | 2228 | 8799 | 1080 | | | | | 27013 | 34877 | |
| 544 | KUV50203.1 | 2232 | 8803 | 1084 | | | | | 16306 | 24182 | |
| 545 | KUT58776.1 | 2236 | 8807 | 1080 | | | | | 39315 | 47179 | |
| 546 | KXL70400.1 | 2240 | 8811 | 1080 | | | | | 1546 | 9410 | |
| 547 | KXM39828.1 | 2244 | 8815 | 1080 | | | | | 6312 | 14176 | |
| 548 | KXM53393.1 | 2248 | 8819 | 1080 | | | | | 2592 | 10456 | |
| 549 | KXN00951.1 | 2252 | 8823 | 1080 | | | | | 4479 | 12343 | |
| 550 | KXQ06779.1 | 2256 | 8827 | 1080 | | | | | 75 | 7939 | |
| 551 | KXQ54356.1 | 2260 | 8831 | 1080 | | | | | 314 | 8178 | |
| 552 | KXR15703.1 | 2264 | 8835 | 1080 | | | | | 50549 | 58413 | |
| 553 | KXQ76403.1 | 2268 | 8839 | 1080 | | | | | 522 | 8386 | |
| 554 | KXQ99886.1 | 2272 | 8843 | 1080 | | | | | 312 | 8176 | |
| 555 | KXR88731.1 | 2276 | 8847 | 1080 | | | | | 238975 | 246839 | |
| 556 | KXR78154.1 | 2280 | 8851 | 1080 | | | | | 8890 | 16754 | |
| 557 | EGC96563.1 | 2284 | 8855 | 1080 | | | | | 3169949 | 3177774 | |
| 558 | GAB54116.1 | 2288 | 8859 | 627 | | | | | 9182 | 15685 | |
| 559 | EHM44107.1 | 2292 | 8863 | 1080 | | | | | 4368 | 12232 | |
| 560 | EKB79931.1 | 2296 | 8867 | 1080 | | | | | 833707 | 841571 | |
| 561 | EKB69924.1 | 2300 | 8871 | 1082 | | | | | 141821 | 149691 | |
| 562 | EMH92795.1 | 2304 | 8875 | 470 | | | | | 56292 | 64102 | |
| 563 | EMH90434.1 | 2308 | 8879 | 1082 | | | | | 2827 | 10697 | |
| 564 | EPA89043.1 | 2312 | 8883 | 1080 | | | | | 31904 | 39684 | |
| 565 | EPO98322.1 | 2316 | 8887 | 1080 | | | | | 3559 | 11339 | |
| 566 | ERN60894.1 | 2320 | 8891 | 1080 | | | | | 58696 | 66506 | |
| 567 | ESM66667.1 | 2324 | 8895 | 1080 | | | | | 3031589 | 3039453 | |
| 568 | | | | | | | | | 7279 | 11205 | |
| 569 | EWE84470.1 | 2331 | 8902 | 1082 | | | | | 156425 | 164295 | |
| 570 | EWE89861.1 | 2335 | 8906 | 1080 | | | | | 59506 | 67370 | |
| 571 | EZQ78639.1 | 2339 | 8910 | 1080 | | | | | 56522 | 64386 | |
| 572 | EZR23961.1 | 2343 | 8914 | 1080 | | | | | 56535 | 64399 | |
| 573 | KDK71113.1 | 2347 | 8918 | 1080 | | | | | 56471 | 64335 | |
| 574 | KDL50058.1 | 2351 | 8922 | 1080 | | | | | 102416 | 110280 | |
| 575 | KEF24234.1 | 2355 | 8926 | 1080 | | | | | 298318 | 306182 | |
| 576 | AIG86118.1 | 2359 | 8930 | 1080 | | | | | 2499874 | 2507684 | |
| 577 | AIT02044.1 | 2363 | 8934 | 1080 | | | | | 2796337 | 2804201 | |
| 578 | KGK55642.1 | 2367 | 8938 | 1080 | | | | | 4976163 | 4984027 | |
| 579 | KHF56288.1 | 2371 | 8942 | 1080 | | | | | 56658 | 64522 | |
| 580 | CEO84462.1 | 2375 | 8946 | 1080 | | | | | 56654 | 64518 | |
| 581 | KLZ85287.1 | 2379 | 8950 | 1080 | | | | | 299767 | 307631 | |
| 582 | KME72629.1 | 2383 | 8954 | 1082 | | | | | 341221 | 349091 | |
| 583 | KMG80270.1 | 2387 | 8958 | 1080 | | | | | 56631 | 64495 | |
| 584 | KMH33141.1 | 2391 | 8962 | 1080 | | | | | 298484 | 306348 | |
| 585 | KMH45009.1 | 2395 | 8966 | 1080 | | | | | 298657 | 306521 | |
| 586 | KMH61877.1 | 2399 | 8970 | 1080 | | | | | 298657 | 306521 | |
| 587 | KMH74808.1 | 2403 | 8974 | 1080 | | | | | 298345 | 306209 | |
| 588 | KMH92294.1 | 2407 | 8978 | 1080 | | | | | 298488 | 306352 | |
| 589 | KMH82196.1 | 2411 | 8982 | 1082 | | | | | 33540 | 41410 | |
| 590 | KMI35041.1 | 2415 | 8986 | 1082 | | | | | 411073 | 418943 | |
| 591 | KMJ00754.1 | 2419 | 8990 | 1080 | | | | | 298445 | 306309 | |
| 592 | KMX47012.1 | 2423 | 8994 | 1080 | | | | | 159517 | 167381 | |
| 593 | ALH85686.1 | 2427 | 8998 | 1080 | | | | | 2553997 | 2561861 | |
| 594 | KPR26028.1 | 2431 | 9002 | 1080 | | | | | 257164 | 265028 | |
| 595 | KSW78887.1 | 2435 | 9006 | 1077 | | | | | 127014 | 135431 | |
| 596 | ALP77149.1 | 2439 | 9010 | 1080 | | | | | 1308820 | 1316684 | |
| 597 | KSZ25784.1 | 2443 | 9014 | 1080 | | | | | 284411 | 292275 | |
| 598 | AMA16469.1 | 2447 | 9018 | 1080 | | | | | 2542623 | 2550487 | |
| 599 | KXO21004.1 | 2451 | 9022 | 1080 | | | | | 28886 | 36750 | |
| 600 | KYL54016.1 | 2455 | 9026 | 1080 | | | | | 10919 | 18783 | |
| 601 | KYM08972.1 | 2459 | 9030 | 1080 | | | | | 14860 | 22724 | |
| 602 | KYL42997.1 | 2463 | 9034 | 1080 | | | | | 14860 | 22724 | |
| 603 | KYL42678.1 | 2467 | 9038 | 1080 | | | | | 14870 | 22734 | |
| 604 | KYL71100.1 | 2471 | 9042 | 1080 | | | | | 10922 | 18786 | |
| 605 | KYL53996.1 | 2475 | 9046 | 1080 | | | | | 10920 | 18784 | |
| 606 | KYM33516.1 | 2479 | 9050 | 1080 | | | | | 284951 | 292815 | |
| 607 | KYL73346.1 | 2483 | 9054 | 1080 | | | | | 14860 | 22724 | |
| 608 | KYL53876.1 | 2487 | 9058 | 1080 | | | | | 14862 | 22726 | |
| 609 | KYM21788.1 | 2491 | 9062 | 1080 | | | | | 14861 | 22725 | |
| 610 | KYM07146.1 | 2495 | 9066 | 1080 | | | | | 10923 | 18787 | |
| 611 | KYM09276.1 | 2499 | 9070 | 1080 | | | | | 10920 | 18784 | |
| 612 | KYL81722.1 | 2503 | 9074 | 1080 | | | | | 14861 | 22725 | |
| 613 | KYL90350.1 | 2507 | 9078 | 1080 | | | | | 14861 | 22725 | |
| 614 | KYM24954.1 | 2511 | 9082 | 1080 | | | | | 10920 | 18784 | |
| 615 | CQJ58396.1 | 2515 | 9086 | 1042 | | | | | 442633 | 449979 | |
| 616 | CFV22979.1 | 2519 | 9090 | 1042 | | | | | 128423 | 135769 | |
| 617 | CNI05674.1 | 2523 | 9094 | 1042 | | | | | 382852 | 390198 | |
| 618 | AHV33809.1 | 2527 | 9098 | 1019 | | | | | 445565 | 453509 | |
| 619 | KOG92698.1 | 2531 | 9102 | 1019 | | | | | 891037 | 898981 | |
| 620 | KRW58409.1 | 2535 | 9106 | 1254 | | | | | 282454 | 290695 | |
| 621 | AAF94909.1 | 2539 | 9110 | 940 | | | | | 1898009 | 1905745 | |
| 622 | ABQ21454.1 | 2543 | 9114 | 1146 | | | | | 1451122 | 1458848 | |
| 623 | ACP06006.1 | 2547 | 9118 | 1149 | | | | | 1845143 | 1852878 | |
| 624 | ACP09871.1 | 2551 | 9122 | 1146 | | | | | 1982541 | 1990267 | |
| 625 | | | | | | | | | 1741763 | 1746058 | |
| 626 | EFH78552.1 | 2558 | 9129 | 1149 | | | | | 406663 | 414398 | |
| 627 | EAZ72426.1 | 2561 | 9132 | 1149 | | | | | 102894 | 108516 | |
| 628 | EAZ78570.1 | 2565 | 9136 | 1149 | | | | | 39155 | 46890 | |
| 629 | AET26856.1 | 2569 | 9140 | 1149 | | | | | 1369410 | 1377145 | |
| 630 | KNH51116.1 | 2573 | 9144 | 1149 | | | | | 31898 | 39633 | |
| 631 | KNA61361.1 | 2577 | 9148 | 1149 | | | | | 426524 | 434259 | |
| 632 | EEO08428.1 | 2581 | 9152 | 932 | | | | | 320173 | 327908 | |
| 633 | EEO16531.1 | 2585 | 9156 | 932 | | | | | 1108223 | 1115958 | |
| 634 | EEO20562.1 | 2589 | 9160 | 932 | | | | | 1025633 | 1033368 | |
| 635 | EET92690.1 | 2593 | 9164 | 1149 | | | | | 31076 | 38811 | |
| 636 | EEY40705.1 | 2597 | 9168 | 1149 | | | | | 318366 | 326101 | |
| 637 | EEY47280.1 | 2601 | 9172 | 1149 | | | | | 293048 | 300726 | |
| 638 | EGQ97274.1 | 2605 | 9176 | 1149 | | | | | 315513 | 323191 | |
| 639 | EGQ98248.1 | 2609 | 9180 | 1149 | | | | | 315860 | 323595 | |
| 640 | EGS48432.1 | 2613 | 9184 | 1149 | | | | | 2498 | 10233 | |
| 641 | EGS47643.1 | 2617 | 9188 | 1149 | | | | | 159058 | 166793 | |
| 642 | EGS47804.1 | 2621 | 9192 | 1149 | | | | | 159009 | 166744 | |
| 643 | EGS62505.1 | 2625 | 9196 | 1149 | | | | | 159057 | 166735 | |
| 644 | EGS70772.1 | 2629 | 9200 | 1149 | | | | | 159054 | 166789 | |
| 645 | EHH71588.1 | 2633 | 9204 | 1149 | | | | | 87128 | 94863 | |
| 646 | EHH83255.1 | 2637 | 9208 | 1149 | | | | | 313471 | 321206 | |
| 647 | EHH87247.1 | 2641 | 9212 | 1149 | | | | | 159015 | 166750 | |
| 648 | EHH97389.1 | 2645 | 9216 | 1149 | | | | | 315855 | 323590 | |
| 649 | EHI04570.1 | 2649 | 9220 | 1149 | | | | | 318768 | 326503 | |
| 650 | EHI04038.1 | 2653 | 9224 | 1149 | | | | | 1 | 6847 | Additional ZorC homolog EHI04039.1 |
| 651 | EHH99554.1 | 2657 | 9228 | 1149 | | | | | 159021 | 166756 | |
| 652 | EHH93198.1 | 2661 | 9232 | 1149 | | | | | 159052 | 166787 | |
| 653 | EHH82142.1 | 2665 | 9236 | 1149 | | | | | 159011 | 166746 | |
| 654 | EHH72364.1 | 2669 | 9240 | 1149 | | | | | 159013 | 166748 | |
| 655 | EHH76757.1 | 2673 | 9244 | 1149 | | | | | 159078 | 166813 | |
| 656 | AFC58579.1 | 2677 | 9248 | 1149 | | | | | 1944318 | 1952053 | |
| 657 | EJH73149.1 | 2681 | 9252 | 1149 | | | | | 159078 | 166813 | |
| 658 | EJH69789.1 | 2685 | 9256 | 1149 | | | | | 159086 | 166821 | |
| 659 | EJH28003.1 | 2689 | 9260 | 1149 | | | | | 159398 | 167133 | |
| 660 | EJH27515.1 | 2693 | 9264 | 1149 | | | | | 159452 | 167187 | |
| 661 | EJH29360.1 | 2697 | 9268 | 1149 | | | | | 314088 | 321823 | |
| 662 | EJH38297.1 | 2701 | 9272 | 1149 | | | | | 311261 | 318996 | |
| 663 | EJH70290.1 | 2705 | 9276 | 1149 | | | | | 159175 | 166910 | |
| 664 | EJH44247.1 | 2709 | 9280 | 1149 | | | | | 313098 | 320833 | |
| 665 | EJH55110.1 | 2713 | 9284 | 1149 | | | | | 1125543 | 1133278 | |
| 666 | EJH55326.1 | 2717 | 9288 | 1149 | | | | | 315841 | 323576 | |
| 667 | EJH83355.1 | 2721 | 9292 | 1149 | | | | | 360461 | 368196 | |
| 668 | EJH85690.1 | 2725 | 9296 | 1149 | | | | | 159055 | 166790 | |
| 669 | EJH80826.1 | 2729 | 9300 | 1149 | | | | | 159198 | 166933 | |
| 670 | EJH40242.1 | 2733 | 9304 | 1149 | | | | | 349947 | 357682 | |
| 671 | EKG51166.1 | 2737 | 9308 | 1149 | | | | | 159143 | 166878 | |
| 672 | EKG45545.1 | 2741 | 9312 | 1149 | | | | | 159094 | 166829 | |
| 673 | EKG69361.1 | 2745 | 9316 | 1149 | | | | | 159020 | 166755 | |
| 674 | EKG78204.1 | 2749 | 9320 | 1149 | | | | | 360413 | 368148 | |
| 675 | EKG79524.1 | 2753 | 9324 | 1149 | | | | | 159046 | 166724 | |
| 676 | EKG89062.1 | 2757 | 9328 | 1149 | | | | | 159114 | 166792 | |
| 677 | EKK80940.1 | 2761 | 9332 | 1149 | | | | | 152818 | 160553 | |
| 678 | EKK97420.1 | 2765 | 9336 | 1149 | | | | | 159065 | 166743 | |
| 679 | EKL07614.1 | 2769 | 9340 | 932 | | | | | 2711 | 8667 | |
| 680 | EKL26686.1 | 2773 | 9344 | 1149 | | | | | 159013 | 166748 | |
| 681 | EKL25947.1 | 2777 | 9348 | 1149 | | | | | 159005 | 166740 | |
| 682 | EKL86047.1 | 2781 | 9352 | 1149 | | | | | 159001 | 166736 | |
| 683 | EKM09898.1 | 2785 | 9356 | 1149 | | | | | 159050 | 166785 | |
| 684 | EKL86443.1 | 2789 | 9360 | 1149 | | | | | 159016 | 166694 | |
| 685 | EKM18413.1 | 2793 | 9364 | 1149 | | | | | 159007 | 166685 | |
| 686 | ELT24498.1 | 2797 | 9368 | 1149 | | | | | 318101 | 325779 | |
| 687 | ELT39814.1 | 2801 | 9372 | 1149 | | | | | 318117 | 325852 | |
| 688 | ELS74164.1 | 2805 | 9376 | 1149 | | | | | 159011 | 166746 | |
| 689 | ELS81595.1 | 2809 | 9380 | 1149 | | | | | 159013 | 166691 | |
| 690 | ELS88994.1 | 2813 | 9384 | 1149 | | | | | 159009 | 166744 | |
| 691 | ELS96625.1 | 2817 | 9388 | 1149 | | | | | 159067 | 166802 | |
| 692 | ELT03891.1 | 2821 | 9392 | 1149 | | | | | 159007 | 166742 | |
| 693 | ELT10274.1 | 2825 | 9396 | 1149 | | | | | 159009 | 166687 | |
| 694 | ELT32498.1 | 2829 | 9400 | 1149 | | | | | 159005 | 166740 | |
| 695 | EMB02077.1 | 2833 | 9404 | 1149 | | | | | 2023763 | 2031498 | |
| 696 | EMP84511.1 | 2837 | 9408 | 1149 | | | | | 159020 | 166755 | |
| 697 | EMP97714.1 | 2841 | 9412 | 1146 | | | | | 159712 | 167381 | |
| 698 | EMP95635.1 | 2845 | 9416 | 1149 | | | | | 159039 | 166774 | |
| 699 | EMQ03271.1 | 2849 | 9420 | 1149 | | | | | 159039 | 166774 | |
| 700 | EMQ04746.1 | 2853 | 9424 | 1149 | | | | | 159015 | 166693 | |
| 701 | EMQ11840.1 | 2857 | 9428 | 1149 | | | | | 159023 | 166701 | |
| 702 | EMQ11494.1 | 2861 | 9432 | 1149 | | | | | 159013 | 166748 | |
| 703 | EMQ16254.1 | 2865 | 9436 | 1149 | | | | | 159021 | 166699 | |
| 704 | EMQ22000.1 | 2869 | 9440 | 1149 | | | | | 159017 | 166752 | |
| 705 | EMQ27290.1 | 2873 | 9444 | 1149 | | | | | 130877 | 138612 | |
| 706 | EMQ26217.1 | 2877 | 9448 | 1149 | | | | | 130881 | 138616 | |
| 707 | EMQ32317.1 | 2881 | 9452 | 1149 | | | | | 159017 | 166752 | |
| 708 | EMQ37897.1 | 2885 | 9456 | 1149 | | | | | 159026 | 166761 | |
| 709 | EMQ51392.1 | 2889 | 9460 | 1149 | | | | | 159021 | 166756 | |
| 710 | EMQ53993.1 | 2893 | 9464 | 1149 | | | | | 159019 | 166754 | |
| 711 | EMQ39195.1 | 2897 | 9468 | 1149 | | | | | 159017 | 166695 | |
| 712 | EMQ68293.1 | 2901 | 9472 | 1149 | | | | | 159017 | 166695 | |
| 713 | EMQ62926.1 | 2905 | 9476 | 1149 | | | | | 159012 | 166690 | |
| 714 | EMQ43436.1 | 2909 | 9480 | 1149 | | | | | 159013 | 166748 | |
| 715 | EMQ47712.1 | 2913 | 9484 | 1149 | | | | | 159021 | 166756 | |
| 716 | EWM32708.1 | 2917 | 9488 | 1149 | | | | | 130877 | 138612 | |
| 717 | KEA49017.1 | 2921 | 9492 | 1149 | | | | | 158099 | 165834 | |
| 718 | | | | | | | | | 327156 | 331451 | |
| 719 | KEH80155.1 | 2928 | 9499 | 1149 | | | | | 31003 | 38738 | |
| 720 | KEH82626.1 | 2932 | 9503 | 1149 | | | | | 31003 | 38738 | |
| 721 | | | | | | | | | 5628 | 9512 | |
| 722 | | | | | | | | | 1490 | 5374 | |
| 723 | KFE03686.1 | 2942 | 9513 | 1149 | | | | | 158948 | 166626 | |
| 724 | KFE20273.1 | 2946 | 9517 | 1149 | | | | | 31824 | 39502 | |
| 725 | KFZ34212.1 | 2950 | 9521 | 1149 | | | | | 158938 | 166673 | |
| 726 | AIT29753.1 | 2954 | 9525 | 1149 | | | | | 1367243 | 1374978 | |
| 727 | KHE18841.1 | 2958 | 9529 | 1149 | | | | | 115674 | 123409 | |
| 728 | KHE21919.1 | 2962 | 9533 | 1149 | | | | | 30978 | 38713 | |
| 729 | BAP03189.1 | 2966 | 9537 | 1149 | | | | | 1752968 | 1760703 | |
| 730 | KJD16374.1 | 2970 | 9541 | 1149 | | | | | 31764 | 39499 | |
| 731 | KJJ52417.1 | 2974 | 9545 | 1149 | | | | | 311822 | 319557 | |
| 732 | KJJ54926.1 | 2978 | 9549 | 1149 | | | | | 311849 | 319584 | |
| 733 | KJR76010.1 | 2982 | 9553 | 1149 | | | | | 158920 | 166655 | |
| 734 | KJS92994.1 | 2986 | 9557 | 1146 | | | | | 159627 | 167353 | |
| 735 | AKA00222.1 | 2990 | 9561 | 1149 | | | | | 1981293 | 1989028 | |
| 736 | KJW14524.1 | 2994 | 9565 | 1149 | | | | | 22906 | 30641 | |
| 737 | KJX83384.1 | 2998 | 9569 | 1149 | | | | | 30976 | 38711 | |
| 738 | KKP13600.1 | 3002 | 9573 | 1149 | | | | | 311137 | 318815 | |
| 739 | KKP11540.1 | 3006 | 9577 | 1149 | | | | | 159049 | 166784 | |
| 740 | KKP10400.1 | 3010 | 9581 | 1149 | | | | | 31066 | 38801 | |
| 741 | AKO75283.1 | 3014 | 9585 | 1149 | | | | | 1873149 | 1880884 | |
| 742 | CSC37318.1 | 3017 | 9588 | 650 | | | | | 33349 | 39027 | |
| 743 | CRZ83750.1 | 3021 | 9592 | 1149 | | | | | 323105 | 330857 | |
| 744 | CSE02169.1 | 3025 | 9596 | 1149 | | | | | 310728 | 318406 | |
| 745 | CRZ49585.1 | 3029 | 9600 | 1149 | | | | | 310734 | 318412 | |
| 746 | CSC16469.1 | 3033 | 9604 | 1149 | | | | | 31070 | 38748 | |
| 747 | CSB69205.1 | 3037 | 9608 | 1149 | | | | | 31121 | 38824 | |
| 748 | CSA28505.1 | 3041 | 9612 | 1146 | | | | | 31851 | 39520 | |
| 749 | CSB84087.1 | 3045 | 9616 | 657 | | | | | 135830 | 143726 | |
| 750 | CSB63121.1 | 3049 | 9620 | 1149 | | | | | 16865 | 24600 | |
| 751 | CSA19206.1 | 3053 | 9624 | 1149 | | | | | 31070 | 38748 | |
| 752 | CSD58145.1 | 3057 | 9628 | 1149 | | | | | 31082 | 38760 | |
| 753 | | | | | | | | | 166125 | 170429 | |
| 754 | CSB47757.1 | 3064 | 9635 | 1149 | | | | | 31012 | 38747 | |
| 755 | CSA30951.1 | 3068 | 9639 | 1146 | | | | | 31850 | 39519 | |
| 756 | CSB56119.1 | 3072 | 9643 | 1149 | | | | | 31737 | 39415 | |
| 757 | CRZ98351.1 | 3076 | 9647 | 1149 | | | | | 365059 | 372737 | |
| 758 | CSA01334.1 | 3080 | 9651 | 1149 | | | | | 17713 | 25417 | |
| 759 | CSB97529.1 | 3084 | 9655 | 1149 | | | | | 159188 | 166980 | |
| 760 | CSC01752.1 | 3088 | 9659 | 1149 | | | | | 159378 | 167056 | |
| 761 | CRZ46109.1 | 3092 | 9663 | 565 | | | | | 31477 | 39390 | |
| 762 | CSA07846.1 | 3096 | 9667 | 1149 | | | | | 361547 | 369225 | |
| 763 | CSC23719.1 | 3100 | 9671 | 1149 | | | | | 159382 | 167060 | |
| 764 | | | | | | | | | 31152 | 35390 | |
| 765 | CSE01568.1 | 3107 | 9678 | 1149 | | | | | 31080 | 38758 | |
| 766 | CSA08428.1 | 3110 | 9681 | 1149 | | | | | 111629 | 119351 | |
| 767 | CRZ45164.1 | 3114 | 9685 | 1149 | | | | | 360008 | 367686 | |
| 768 | CSA07018.1 | 3118 | 9689 | 1149 | | | | | 31070 | 38748 | |
| 769 | CSA08758.1 | 3122 | 9693 | 1149 | | | | | 311375 | 319053 | |
| 770 | CSA15617.1 | 3126 | 9697 | 1151 | | | | | 118328 | 126027 | |
| 771 | CSA74554.1 | 3130 | 9701 | 421 | | | | | 31048 | 38213 | |
| 772 | CSB88604.1 | 3133 | 9704 | 650 | | | | | 202946 | 208618 | |
| 773 | CSC80153.1 | 3137 | 9708 | 1149 | | | | | 158978 | 166656 | |
| 774 | CSB83805.1 | 3141 | 9712 | 1146 | | | | | 33081 | 40750 | |
| 775 | CSB56566.1 | 3144 | 9715 | 902 | | | | | 33322 | 38967 | |
| 776 | CSC85417.1 | 3148 | 9719 | 1149 | | | | | 49583 | 57261 | |
| 777 | CSB86278.1 | 3152 | 9723 | 327 | | | | | 79066 | 86933 | |
| 778 | CSD13130.1 | 3156 | 9727 | 1149 | | | | | 31083 | 38761 | |
| 779 | CSD79384.1 | 3160 | 9731 | 1149 | | | | | 158961 | 166639 | |
| 780 | CSD86903.1 | 3164 | 9735 | 1149 | | | | | 311074 | 318752 | |
| 781 | CSA39718.1 | 3168 | 9739 | 1149 | | | | | 31077 | 38755 | |
| 782 | CSC76671.1 | 3172 | 9743 | 1149 | | | | | 159318 | 166996 | |
| 783 | CSA42827.1 | 3176 | 9747 | 1149 | | | | | 17305 | 24983 | |
| 784 | CSA52168.1 | 3180 | 9751 | 1149 | | | | | 31069 | 38747 | |
| 785 | CSD31842.1 | 3184 | 9755 | 1149 | | | | | 310993 | 318671 | |
| 786 | CSD17868.1 | 3188 | 9759 | 1149 | | | | | 31086 | 38764 | |
| 787 | CSD38685.1 | 3192 | 9763 | 1149 | | | | | 310723 | 318401 | |
| 788 | CSA26333.1 | 3196 | 9767 | 1149 | | | | | 159373 | 167051 | |
| 789 | CSA45918.1 | 3200 | 9771 | 1149 | | | | | 31045 | 38780 | |
| 790 | CSD96324.1 | 3204 | 9775 | 1149 | | | | | 31082 | 38760 | |
| 791 | CSA54003.1 | 3208 | 9779 | 1149 | | | | | 159718 | 167396 | |
| 792 | CSC37805.1 | 3211 | 9782 | 233 | | | | | 32052 | 39355 | |
| 793 | CSA88183.1 | 3215 | 9786 | 413 | | | | | 33049 | 40215 | |
| 794 | CSA15198.1 | 3219 | 9790 | 496 | | | | | 31993 | 39834 | |
| 795 | CSC98525.1 | 3222 | 9793 | 327 | | | | | 34368 | 40014 | |
| 796 | CRZ48549.1 | 3226 | 9797 | 1157 | | | | | 160331 | 168052 | |
| 797 | CSB25047.1 | 3229 | 9800 | 435 | | | | | 34141 | 39160 | Additional ZorC homolog CSB25035.1 |
| 798 | CSD98674.1 | 3233 | 9804 | 1149 | | | | | 360011 | 367689 | |
| 799 | CSA36985.1 | 3236 | 9807 | 650 | | | | | 33493 | 39175 | |
| 800 | | | | | | | | | 32082 | 36377 | |
| 801 | CSD70475.1 | 3243 | 9814 | 1149 | | | | | 31083 | 38761 | |
| 802 | CSD13890.1 | 3246 | 9817 | 657 | | | | | 459 | 6109 | |
| 803 | CSD31883.1 | 3250 | 9821 | 1149 | | | | | 31084 | 38762 | |
| 804 | CSE18808.1 | 3254 | 9825 | 1149 | | | | | 158956 | 166634 | |
| 805 | CSD93195.1 | 3258 | 9829 | 1149 | | | | | 31084 | 38762 | |
| 806 | CSA21132.1 | 3262 | 9833 | 1149 | | | | | 159994 | 167672 | |
| 807 | CSA44698.1 | 3266 | 9837 | 1146 | | | | | 160570 | 168345 | |
| 808 | CSD30628.1 | 3270 | 9841 | 1149 | | | | | 207665 | 215343 | |
| 809 | CSD92385.1 | 3274 | 9845 | 1149 | | | | | 310736 | 318414 | |
| 810 | CSD99314.1 | 3278 | 9849 | 1149 | | | | | 31871 | 39549 | |
| 811 | CSA22955.1 | 3282 | 9853 | 1149 | | | | | 159775 | 167453 | |
| 812 | CSA49214.1 | 3286 | 9857 | 366 | | | | | 31257 | 39041 | |
| 813 | CSC63613.1 | 3290 | 9861 | 650 | | | | | 9409 | 16634 | |
| 814 | CRZ49258.1 | 3294 | 9865 | 1149 | | | | | 33478 | 41156 | |
| 815 | CSC03090.1 | 3298 | 9869 | 1149 | | | | | 124696 | 132431 | |
| 816 | CSD87248.1 | 3302 | 9873 | 1149 | | | | | 310735 | 318413 | |
| 817 | CSD21248.1 | 3306 | 9877 | 1149 | | | | | 388072 | 395750 | |
| 818 | | | | | | | | | 31912 | 36177 | |
| 819 | CSA26743.1 | 3313 | 9884 | 1149 | | | | | 159358 | 167090 | |
| 820 | CSB50330.1 | 3317 | 9888 | 1149 | | | | | 668438 | 676116 | |
| 821 | CSB24819.1 | 3321 | 9892 | 233 | | | | | 32013 | 39918 | |
| 822 | CSB68067.1 | 3325 | 9896 | 650 | | | | | 31070 | 38775 | |
| 823 | CSB16272.1 | 3329 | 9900 | 915 | | | | | 333817 | 341563 | |
| 824 | CRZ89203.1 | 3333 | 9904 | 1149 | | | | | 310756 | 318434 | |
| 825 | CSD99947.1 | 3337 | 9908 | 1149 | | | | | 31875 | 39553 | |
| 826 | CSA40881.1 | 3341 | 9912 | 1149 | | | | | 31156 | 38834 | |
| 827 | CSC19347.1 | 3345 | 9916 | 1149 | | | | | 310775 | 318453 | |
| 828 | CSD27143.1 | 3349 | 9920 | 1149 | | | | | 158972 | 166650 | |
| 829 | CSA25008.1 | 3353 | 9924 | 1149 | | | | | 159300 | 166978 | |
| 830 | CSD33007.1 | 3357 | 9928 | 1149 | | | | | 26879 | 34089 | |
| 831 | CSD25943.1 | 3361 | 9932 | 1149 | | | | | 311072 | 318750 | |
| 832 | CSD37937.1 | 3365 | 9936 | 1149 | | | | | 310737 | 318415 | |
| 833 | CSD14752.1 | 3369 | 9940 | 1149 | | | | | 31084 | 38762 | |
| 834 | | | | | | | | | 162427 | 166665 | |
| 835 | CSD47052.1 | 3376 | 9947 | 1149 | | | | | 31087 | 38765 | |
| 836 | CRZ80385.1 | 3380 | 9951 | 1149 | | | | | 313075 | 320840 | |
| 837 | CSC19211.1 | 3384 | 9955 | 1149 | | | | | 168078 | 175756 | |
| 838 | CSA23993.1 | 3388 | 9959 | 1146 | | | | | 160208 | 167877 | |
| 839 | CSA08080.1 | 3392 | 9963 | 1149 | | | | | 31862 | 39540 | |
| 840 | CSB55683.1 | 3396 | 9967 | 797 | | | | | 278315 | 286089 | |
| 841 | CSA25866.1 | 3400 | 9971 | 1149 | | | | | 31850 | 39528 | |
| 842 | CSD34664.1 | 3404 | 9975 | 1149 | | | | | 310731 | 318409 | |
| 843 | CSC58104.1 | 3408 | 9979 | 1146 | | | | | 63231 | 70982 | |
| 844 | CSA23928.1 | 3412 | 9983 | 817 | | | | | 31012 | 38784 | |
| 845 | CSB25832.1 | 3416 | 9987 | 239 | | | | | 4488 | 12028 | |
| 846 | CSD08425.1 | 3420 | 9991 | 1149 | | | | | 310632 | 318310 | |
| 847 | CSD25614.1 | 3424 | 9995 | 1149 | | | | | 31075 | 38753 | |
| 848 | CSD28595.1 | 3428 | 9999 | 1149 | | | | | 31079 | 38757 | |
| 849 | CSA45233.1 | 3432 | 10003 | 1146 | | | | | 160096 | 167765 | |
| 850 | CSD82509.1 | 3436 | 10007 | 1149 | | | | | 360009 | 367687 | |
| 851 | CSC20734.1 | 3440 | 10011 | 1149 | | | | | 159381 | 167059 | |
| 852 | CSC43362.1 | 3444 | 10015 | 902 | | | | | 63491 | 71271 | |
| 853 | CSC06923.1 | 3448 | 10019 | 1149 | | | | | 31021 | 38756 | |
| 854 | CSB50482.1 | 3452 | 10023 | 1149 | | | | | 177974 | 185709 | |
| 855 | CSE01128.1 | 3456 | 10027 | 1149 | | | | | 310979 | 318657 | |
| 856 | CSB99370.1 | 3460 | 10031 | 1149 | | | | | 31077 | 38755 | |
| 857 | KOY92949.1 | 3463 | 10034 | 1149 | | | | | 212064 | 217686 | |
| 858 | KOY92341.1 | 3467 | 10038 | 1149 | | | | | 159277 | 167012 | |
| 859 | KOY97237.1 | 3471 | 10042 | 1149 | | | | | 12401 | 20136 | |
| 860 | KOZ01029.1 | 3475 | 10046 | 1149 | | | | | 211986 | 219721 | |
| 861 | KPA03324.1 | 3479 | 10050 | 1149 | | | | | 360412 | 368147 | |
| 862 | ALJ63819.1 | 3483 | 10054 | 1149 | | | | | 1178223 | 1185958 | |
| 863 | KTL56322.1 | 3487 | 10058 | 1146 | | | | | 324982 | 332708 | |
| 864 | KUO35495.1 | 3490 | 10061 | 1149 | | | | | 158946 | 164568 | |
| 865 | | | | | | | | | 30980 | 35275 | |
| 866 | KUP86909.1 | 3497 | 10068 | 1149 | | | | | 30979 | 38714 | |
| 867 | KUY44288.1 | 3501 | 10072 | 1149 | | | | | 30985 | 38720 | |
| 868 | KVI44902.1 | 3505 | 10076 | 1149 | | | | | 343662 | 351397 | |
| 869 | KWW48036.1 | 3509 | 10080 | 1149 | | | | | 31013 | 38748 | |
| 870 | EIG23745.1 | 3513 | 10084 | 999 | | | | | 100888 | 108391 | |
| 871 | KGQ46275.1 | 3517 | 10088 | 1033 | | | | | 80629 | 88171 | |
| 872 | KGQ51099.1 | 3521 | 10092 | 1033 | | | | | 57777 | 65324 | |
| 873 | KGQ65034.1 | 3525 | 10096 | 1033 | | | | | 22663 | 30210 | |
| 874 | ACX76636.1 | 3529 | 10100 | 1045 | | | | | 3759682 | 3766672 | |
| 875 | ADL25841.1 | 3533 | 10104 | 1045 | | | | | 363595 | 370585 | |
| 876 | EEO43109.1 | 3537 | 10108 | 1163 | | | | | 2128114 | 2135123 | |
| 877 | EHG17833.2 | 3541 | 10112 | 1163 | | | | | 2126049 | 2133058 | |
| 878 | ERT42788.1 | 3545 | 10116 | 1163 | | | | | 50872 | 58025 | |
| 879 | KGE62644.1 | 3549 | 10120 | 1162 | | | | | 397783 | 404930 | |
| 880 | EFE87097.1 | 3553 | 10124 | 1166 | | | | | 770761 | 777911 | |
| 881 | ABM29950.1 | 3557 | 10128 | 1131 | | | | | 3435271 | 3442881 | |
| 882 | ADB47621.1 | 3561 | 10132 | 969 | | | | | 1390210 | 1397088 | |
| 883 | ADE85049.1 | 3565 | 10136 | 1030 | | | | | 1376580 | 1384055 | |
| 884 | ETD02076.1 | 3569 | 10140 | 1030 | | | | | 56033 | 63508 | |
| 885 | ETD86669.1 | 3573 | 10144 | 1029 | | | | | 52380 | 59851 | |
| 886 | ETD77750.1 | 3577 | 10148 | 1030 | | | | | 226246 | 233721 | |
| 887 | ETE54108.1 | 3581 | 10152 | 1030 | | | | | 56001 | 63476 | |
| 888 | | | | | | | | | 130723 | 134953 | |
| 889 | KFI23092.1 | 3588 | 10159 | 1150 | | | | | 38137 | 45876 | |
| 890 | KMY56663.1 | 3592 | 10163 | 874 | | | | | 14053 | 20516 | |
| 891 | KMY64906.1 | 3596 | 10167 | 874 | | | | | 14041 | 20504 | |
| 892 | KYD35335.1 | 3600 | 10171 | 860 | | | | | 7802 | 14262 | |
| 893 | GAD13405.1 | 3604 | 10175 | 860 | | | | | 26441 | 32340 | |
| 894 | EMT39962.1 | 3607 | 10178 | 931 | | | | | 147491 | 153762 | |
| 895 | | | | | | | | | 189427 | 193818 | |
| 896 | EKF49052.1 | 3613 | 10184 | 832 | | | | | 59928 | 66237 | Gene EKF49051.1 |
| 897 | BAL95141.1 | 3617 | 10188 | 1287 | | | | | 1873936 | 1882199 | |
| 898 | KTC75264.1 | 3621 | 10192 | 574 | | | | | 10108 | 15843 | |
| 899 | EFF49968.1 | 3625 | 10196 | 221 | | | | | 66300 | 71522 | |
| 900 | ABU75894.1 | 3629 | 10200 | 1080 | | | | | 573812 | 581682 | |
| 901 | EGL73324.1 | 3632 | 10203 | 1080 | | | | | 1391 | 7059 | |
| 902 | | | | | | | | | 3085383 | 3088601 | |
| 903 | CCK02757.1 | 3639 | 10210 | 1080 | | | | | 1992732 | 2000383 | |
| 904 | CCO48378.1 | 3643 | 10214 | 1149 | | | | | 351151 | 358883 | |
| 905 | CCK80599.1 | 3647 | 10218 | 1149 | | | | | 2847791 | 2855375 | |
| 906 | KGD34413.1 | 3651 | 10222 | 961 | | | | | 604039 | 612320 | |
| 907 | KGS74755.1 | 3655 | 10226 | 1253 | | | | | 44290 | 52434 | |
| 908 | KGW09385.1 | 3659 | 10230 | 1257 | | | | | 3032122 | 3040259 | |
| 909 | KGX47861.1 | 3663 | 10234 | 881 | | | | | 14326 | 22286 | |
| 910 | KGX39521.1 | 3667 | 10238 | 1290 | | | | | 2107525 | 2115821 | |
| 911 | CPH56590.1 | 3671 | 10242 | 1258 | | | | | 30629 | 39310 | |
| 912 | KSB52474.1 | 3675 | 10246 | 1080 | | | | | 185515 | 193379 | |
| 913 | AEF17802.1 | 3678 | 10249 | 930 | | | | | 1842826 | 1849094 | |
| 914 | ENW17899.1 | 3682 | 10253 | 1067 | | | | | 59747 | 65556 | |
| 915 | ABO66650.1 | 3686 | 10257 | 874 | | | | | 1352626 | 1359020 | |
| 916 | | | | | | | | | 2415017 | 2419718 | |
| 917 | ACS21191.1 | 3693 | 10264 | 1291 | | | | | 4851523 | 4859825 | |
| 918 | | | | | | | | | 1418094 | 1422180 | |
| 919 | EET81940.1 | 3700 | 10271 | 1067 | | | | | 99524 | 106967 | |
| 920 | ENU31374.1 | 3704 | 10275 | 1067 | | | | | 191572 | 197836 | |
| 921 | ALA59736.1 | 3708 | 10279 | 1244 | | | | | 3145019 | 3153095 | |
| 922 | AGB41058.1 | 3712 | 10283 | 1172 | | | | | 1097125 | 1104635 | |
| 923 | AEI93762.1 | 3716 | 10287 | 1058 | | | | | 1813459 | 1820361 | |
| 924 | CCU78670.1 | 3720 | 10291 | 939 | | | | | 785072 | 791847 | |
| 925 | ERG17895.1 | 3724 | 10295 | 1152 | | | | | 22799 | 30533 | |
| 926 | ALF60589.1 | 3728 | 10299 | 1075 | | | | | 2700909 | 2708350 | |
| 927 | AEM78248.1 | 3731 | 10302 | 931 | | | | | 840832 | 847102 | |
| 928 | EFV93690.1 | 3735 | 10306 | 1109 | | | | | 3038718 | 3046540 | |
| 929 | KRI14523.1 | 3739 | 10310 | 1068 | | | | | 149243 | 156664 | |
| 930 | AEJ38285.1 | 3743 | 10314 | 908 | | | | | 54168 | 60713 | |
| 931 | AEW03627.1 | 3747 | 10318 | 908 | | | | | 54408 | 60953 | |
| 932 | KFX14694.1 | 3751 | 10322 | 1080 | | | | | 19071 | 26935 | |
| 933 | KKW93257.1 | 3755 | 10326 | 1274 | | | | | 127747 | 135625 | |
| 934 | KVX03058.1 | 3759 | 10330 | 1007 | | | | | 67700 | 75395 | |
| 935 | KJM54477.1 | 3763 | 10334 | 1079 | | | | | 76651 | 84984 | |
| 936 | KLF88327.1 | 3767 | 10338 | 1080 | | | | | 57307 | 65172 | |
| 937 | KLG16013.1 | 3771 | 10342 | 1079 | | | | | 39524 | 47386 | |
| 938 | KLP43533.1 | 3775 | 10346 | 1080 | | | | | 57204 | 65069 | |
| 939 | KTK05047.1 | 3779 | 10350 | 1079 | | | | | 80895 | 89228 | |
| 940 | KTJ30245.1 | 3783 | 10354 | 1079 | | | | | 10225 | 18087 | |
| 941 | KTI23425.1 | 3787 | 10358 | 1079 | | | | | 288377 | 296710 | |
| 942 | AKL12334.1 | 3791 | 10362 | 1080 | | | | | 2215142 | 2223007 | |
| 943 | | | | | | | | | 1907323 | 1912534 | |
| 944 | | | | | | | | | 4313226 | 4317455 | |
| 945 | AJG24949.1 | 3801 | 10372 | 1288 | | | | | 3725247 | 3733546 | |
| 946 | | | | | | | | | 8581 | 12602 | |
| 947 | | | | | | | | | 3803428 | 3807386 | |
| 948 | AJE15426.1 | 3811 | 10382 | 1083 | | | | | 2249957 | 2257829 | |
| 949 | | | | | | | | | 869 | 4909 | |
| 950 | EHO11884.1 | 3818 | 10389 | 1089 | | | | | 1267870 | 1274810 | |
| 951 | CAY72980.1 | 3822 | 10393 | 1081 | | | | | 680846 | 688730 | |
| 952 | CAX54370.1 | 3826 | 10397 | 1081 | | | | | 680824 | 688708 | |
| 953 | AGA90550.1 | 3830 | 10401 | 966 | | | | | 1965119 | 1972029 | |
| 954 | CUH46422.1 | 3834 | 10405 | 1042 | | | | | 70525 | 78149 | |
| 955 | AMO80631.1 | 3838 | 10409 | 1082 | | | | | 1259869 | 1267751 | |
| 956 | AGP47381.1 | 3842 | 10413 | 1042 | | | | | 4767443 | 4774474 | |
| 957 | KIN78153.1 | 3846 | 10417 | 1032 | | | | | 1354 | 9819 | |
| 958 | AMS40140.1 | 3850 | 10421 | 1000 | | | | | 1255627 | 1262896 | |
| 959 | ABK50006.1 | 3854 | 10425 | 1148 | | | | | 4544823 | 4552542 | |
| 960 | CAP40553.1 | 3858 | 10429 | 1246 | | | | | 241480 | 249729 | |
| 961 | ERI29927.1 | 3862 | 10433 | 501 | | | | | 71963 | 77851 | |
| 962 | EPZ87097.1 | 3866 | 10437 | 1253 | | | | | 687271 | 695415 | |
| 963 | KKI79918.1 | 3870 | 10441 | 1221 | | | | | 2182489 | 2190633 | |
| 964 | KVD94187.1 | 3874 | 10445 | 1253 | | | | | 12091 | 20284 | |
| 965 | KVT83336.1 | 3878 | 10449 | 1288 | | | | | 78180 | 86479 | |
| 966 | KVW62587.1 | 3882 | 10453 | 1288 | | | | | 3262 | 11561 | |
| 967 | | | | | | | | | 4322511 | 4327158 | |
| 968 | ADD01960.1 | 3888 | 10459 | 931 | | | | | 714604 | 720875 | |
| 969 | AEG43647.1 | 3892 | 10463 | 1005 | | | | | 936023 | 943033 | |
| 970 | ACY51402.1 | 3896 | 10467 | 1149 | | | | | 1803262 | 1810997 | |
| 971 | EDN58082.1 | 3900 | 10471 | 1149 | | | | | 98875 | 106610 | |
| 972 | AIR88229.1 | 3904 | 10475 | 1082 | | | | | 609086 | 616908 | |
| 973 | ABC30367.1 | 3908 | 10479 | 1003 | | | | | 3762737 | 3770015 | |
| 974 | ALV90956.1 | 3912 | 10483 | 1083 | | | | | 367721 | 375594 | |
| 975 | EDL49430.1 | 3916 | 10487 | 1270 | | | | | 30300 | 38162 | |
| 976 | CAQ68799.1 | 3920 | 10491 | 1097 | | | | | 858781 | 866499 | |
| 977 | BAH37230.1 | 3924 | 10495 | 1150 | | | | | 220564 | 228082 | |
| 978 | KGU86623.1 | 3928 | 10499 | 1082 | | | | | 16946 | 24768 | |
| 979 | AFE11186.1 | 3931 | 10502 | 858 | | | | | 3778679 | 3785329 | |
| 980 | EHJ60588.1 | 3935 | 10506 | 1271 | | | | | 82585 | 90450 | |
| 981 | AIT79634.1 | 3939 | 10510 | 1271 | | | | | 1565213 | 1573078 | |
| 982 | | | | | | | | | 1479567 | 1484303 | |
| 983 | ADP10189.1 | 3946 | 10517 | 1081 | | | | | 558724 | 566608 | |
| 984 | ABM39547.1 | 3950 | 10521 | 1259 | | | | | 145574 | 154312 | |
| 985 | EIL89343.1 | 3954 | 10525 | 1080 | | | | | 112461 | 119985 | |
| 986 | GAC31731.1 | 3958 | 10529 | 1156 | | | | | 31246 | 38984 | |
| 987 | | | | | | | | | 2144456 | 2148780 | |
| 988 | KFK00372.1 | 3965 | 10536 | 1080 | | | | | 91457 | 99321 | |
| 989 | CDT04312.1 | 3969 | 10540 | 1148 | | | | | 483924 | 491657 | |
| 990 | CDT11724.1 | 3973 | 10544 | 1148 | | | | | 106517 | 114250 | |
| 991 | KXV35458.1 | 3977 | 10548 | 1028 | | | | | 11957 | 19254 | |
| 992 | EAT06419.1 | 3981 | 10552 | 952 | | | | | 18325 | 29199 | |
| 993 | BAQ44677.1 | 3985 | 10556 | 1033 | | | | | 1242440 | 1249828 | |
| 994 | CCE22487.1 | 3989 | 10560 | 1003 | | | | | 864299 | 871500 | |
| 995 | CEG51219.1 | 3993 | 10564 | 1012 | | | | | 170325 | 178733 | |
| 996 | EJI89229.1 | 3997 | 10568 | 390 | | | | | 559407 | 565225 | |
| 997 | KDE37031.1 | 4001 | 10572 | 1081 | | | | | 305795 | 313692 | |
| 998 | | | | | | | | | 2602720 | 2606694 | |
| 999 | GAE46383.1 | 4007 | 10578 | 810 | | | | | 27879 | 34299 | |
| 1000 | EAQ47299.1 | 4011 | 10582 | 1032 | | | | | 3319389 | 3327854 | |
| 1001 | EDP59416.1 | 4015 | 10586 | 1149 | | | | | 217393 | 225128 | |
| 1002 | CUH52009.1 | 4019 | 10590 | 1071 | | | | | 33047 | 40102 | |
| 1003 | CVK01153.1 | 4023 | 10594 | 1256 | | | | | 2323 | 11357 | |
| 1004 | ABE74737.1 | 4027 | 10598 | 1075 | | | | | 1137832 | 1145312 | |
| 1005 | CCW17773.1 | 4030 | 10601 | 1251 | | | | | 59 | 7588 | |
| 1006 | | | | | | | | | 4213818 | 4218168 | |
| 1007 | AFU46479.1 | 4037 | 10608 | 1093 | | | | | 3031899 | 3040069 | |
| 1008 | EZI30548.1 | 4041 | 10612 | 1082 | | | | | 320199 | 328021 | |
| 1009 | | | | | | | | | 1174385 | 1178645 | |
| 1010 | GAA77180.1 | 4048 | 10619 | 1151 | | | | | 153172 | 160898 | |
| 1011 | AMK16853.1 | 4052 | 10623 | 1074 | | | | | 479754 | 487343 | |
| 1012 | ADQ06818.1 | 4055 | 10626 | 939 | | | | | 1109840 | 1116255 | |
| 1013 | EFN90476.1 | 4059 | 10630 | 1090 | | | | | 8688 | 15379 | |
| 1014 | KXB76240.1 | 4062 | 10633 | 255 | | | | | 20 | 2822 | |
| 1015 | EPR18408.1 | 4066 | 10637 | 441 | | | | | 4420 | 10039 | |
| 1016 | KMO18104.1 | 4070 | 10641 | 968 | | | | | 1639 | 9617 | |
| 1017 | | | | | | | | | 3802 | 7852 | |
| 1018 | ACT17073.1 | 4077 | 10648 | 1248 | | | | | 1186285 | 1194417 | |
| 1019 | KMW37146.1 | 4081 | 10652 | 916 | | | | | 352471 | 359778 | |
| 1020 | CUB00796.1 | 4084 | 10655 | 1032 | | | | | 1448 | 6835 | |
| 1021 | ADJ28734.1 | 4088 | 10659 | 1145 | | | | | 2071841 | 2079565 | |
| 1022 | AJR24434.1 | 4091 | 10662 | 1074 | | | | | 2738468 | 2744069 | |
| 1023 | AJR25290.1 | 4095 | 10666 | 1073 | | | | | 3840140 | 3847708 | |
| 1024 | EHO63913.1 | 4099 | 10670 | 1088 | | | | | 191966 | 200316 | |
| 1025 | KVM95914.1 | 4103 | 10674 | 1250 | | | | | 212451 | 220642 | |
| 1026 | EDY06974.1 | 4107 | 10678 | 874 | | | | | 313023 | 319483 | |
| 1027 | | | | | | | | | 2634211 | 2638780 | |
| 1028 | EHR42624.1 | 4114 | 10685 | 986 | | | | | 67071 | 74599 | |
| 1029 | EHD22820.1 | 4118 | 10689 | 1083 | | | | | 3735867 | 3743740 | |
| 1030 | CRI66751.1 | 4122 | 10693 | 1249 | | | | | 5085246 | 5093439 | |
| 1031 | EFY05646.1 | 4126 | 10697 | 1109 | | | | | 5476 | 13083 | |
| 1032 | EKN30944.1 | 4130 | 10701 | 916 | | | | | 354487 | 361794 | |
| 1033 | KPL51800.1 | 4134 | 10705 | 1004 | | | | | 1329326 | 1336639 | |
| 1034 | AMN39056.1 | 4137 | 10708 | 960 | | | | | 660425 | 667875 | |
| 1035 | KMV29897.1 | 4141 | 10712 | 927 | | | | | 126899 | 133697 | |
| 1036 | CCA91257.1 | 4145 | 10716 | 1170 | | | | | 303090 | 310618 | |
| 1037 | CCA92390.1 | 4149 | 10720 | 1271 | | | | | 1576101 | 1583966 | |
| 1038 | ADE38424.1 | 4153 | 10724 | 1130 | | | | | 185251 | 192804 | |
| 1039 | EPR27901.1 | 4157 | 10728 | 666 | | | | | 4547 | 11006 | |
| 1040 | KYC33909.1 | 4161 | 10732 | 1073 | | | | | 68665 | 76233 | |
| 1041 | | | | | | | | | 3008592 | 3012561 | |
| 1042 | EPR43454.1 | 4168 | 10739 | 962 | | | | | 39663 | 46452 | |
| 1043 | ESU31840.1 | 4171 | 10742 | 886 | | | | | 2112774 | 2119197 | |
| 1044 | KHJ69398.1 | 4175 | 10746 | 1083 | | | | | 29299 | 37172 | |
| 1045 | ERO56338.1 | 4179 | 10750 | 1080 | | | | | 712736 | 720606 | |
| 1046 | KHN63197.1 | 4183 | 10754 | 1081 | | | | | 41108 | 48981 | |
| 1047 | | | | | | | | | 24422 | 28518 | |
| 1048 | CUB04081.1 | 4190 | 10761 | 1008 | | | | | 169304 | 176741 | |
| 1049 | EJM18416.1 | 4194 | 10765 | 1082 | | | | | 19009 | 26828 | |
| 1050 | EUB73629.1 | 4198 | 10769 | 1009 | | | | | 891473 | 899518 | |
| 1051 | | | | | | | | | 80 | 4289 | |
| 1052 | EOU44973.1 | 4205 | 10776 | 1079 | | | | | 2232245 | 2240572 | |
| 1053 | ELC22343.1 | 4209 | 10780 | 1079 | | | | | 132985 | 141312 | |
| 1054 | EOU82995.1 | 4213 | 10784 | 1079 | | | | | 1010570 | 1018897 | |
| 1055 | KXU33091.1 | 4217 | 10788 | 1073 | | | | | 97745 | 105313 | |
| 1056 | EOV44199.1 | 4221 | 10792 | 1079 | | | | | 2185792 | 2194119 | |
| 1057 | EOQ55530.1 | 4225 | 10796 | 1080 | | | | | 932985 | 940849 | |
| 1058 | EOV91300.1 | 4229 | 10800 | 1079 | | | | | 1475831 | 1484158 | |
| 1059 | EOW68212.1 | 4233 | 10804 | 1079 | | | | | 141883 | 150210 | |
| 1060 | AFY17879.1 | 4237 | 10808 | 1011 | | | | | 795376 | 803717 | |
| 1061 | KHK57377.1 | 4241 | 10812 | 1254 | | | | | 222685 | 230903 | |
| 1062 | ENW78278.1 | 4245 | 10816 | 1067 | | | | | 453952 | 461376 | |
| 1063 | EME68213.1 | 4249 | 10820 | 1120 | | | | | 587 | 8575 | |
| 1064 | CDE94531.1 | 4253 | 10824 | 972 | | | | | 1395 | 8284 | |
| 1065 | CDA50131.1 | 4257 | 10828 | 1097 | | | | | 3567 | 10917 | |
| 1066 | ESQ90331.1 | 4261 | 10832 | 1186 | | | | | 26132 | 33751 | |
| 1067 | ESQ74517.1 | 4265 | 10836 | 1252 | | | | | 23941 | 31878 | |
| 1068 | AII86802.1 | 4269 | 10840 | 1040 | | | | | 1362274 | 1370579 | |
| 1069 | KPQ04146.1 | 4273 | 10844 | 1149 | | | | | 804600 | 812315 | |
| 1070 | CUS49189.1 | 4277 | 10848 | 1149 | | | | | 2278030 | 2285745 | |
| 1071 | ESN27962.1 | 4281 | 10852 | 1080 | | | | | 61729 | 69537 | |
| 1072 | EPJ56198.1 | 4285 | 10856 | 994 | | | | | 22834 | 30573 | |
| 1073 | EQA99139.1 | 4289 | 10860 | 1250 | | | | | 233840 | 241719 | |
| 1074 | KMS61418.1 | 4293 | 10864 | 1250 | | | | | 3101463 | 3109342 | |
| 1075 | AGT31735.1 | 4297 | 10868 | 865 | | | | | 1327845 | 1334314 | |
| 1076 | AIY65735.1 | 4301 | 10872 | 1152 | | | | | 2507239 | 2514925 | |
| 1077 | KXW55385.1 | 4305 | 10876 | 1331 | | | | | 74261 | 82426 | |
| 1078 | AKU92630.1 | 4309 | 10880 | 920 | | | | | 3541455 | 3548134 | |
| 1079 | | | | | | | | | 1 | 3561 | |
| 1080 | ERS83849.1 | 4315 | 10886 | 545 | | | | | 18505 | 26132 | |
| 1081 | KXU87123.1 | 4319 | 10890 | 1257 | | | | | 16810 | 25813 | |
| 1082 | KLU37748.1 | 4323 | 10894 | 1284 | | | | | 146865 | 155158 | |
| 1083 | EUC70009.1 | 4327 | 10898 | 1004 | | | | | 47366 | 54537 | |
| 1084 | EXI82586.1 | 4331 | 10902 | 1250 | | | | | 64911 | 73149 | |
| 1085 | KAK42946.1 | 4335 | 10906 | 1102 | | | | | 16857 | 25146 | |
| 1086 | BAO84443.1 | 4339 | 10910 | 1255 | | | | | 2386040 | 2394279 | |
| 1087 | CQR46608.1 | 4342 | 10913 | 898 | | | | | 914451 | 920975 | |
| 1088 | BAP87808.1 | 4346 | 10917 | 1256 | | | | | 715557 | 723918 | |
| 1089 | KDP87794.1 | 4350 | 10921 | 1097 | | | | | 19414 | 27256 | |
| 1090 | KQB03826.1 | 4354 | 10925 | 1149 | | | | | 16315 | 24050 | |
| 1091 | KQA99547.1 | 4358 | 10929 | 1149 | | | | | 159021 | 166756 | |
| 1092 | KEY85733.1 | 4362 | 10933 | 1082 | | | | | 94535 | 102357 | |
| 1093 | GAK41826.1 | 4366 | 10937 | 917 | | | | | 154548 | 161205 | |
| 1094 | KVV58317.1 | 4370 | 10941 | 1263 | | | | | 7829 | 16059 | |
| 1095 | KVX28282.1 | 4374 | 10945 | 1263 | | | | | 7829 | 16059 | |
| 1096 | KVZ97544.1 | 4378 | 10949 | 1250 | | | | | 209019 | 217210 | |
| 1097 | AJY51018.1 | 4382 | 10953 | 1010 | | | | | 2719272 | 2726819 | |
| 1098 | CDX33072.1 | 4386 | 10957 | 992 | | | | | 46644 | 54080 | |
| 1099 | CDX43769.1 | 4390 | 10961 | 992 | | | | | 2178 | 9614 | |
| 1100 | KJS80659.1 | 4393 | 10964 | 1169 | | | | | 21617 | 29198 | |
| 1101 | KGD90265.1 | 4397 | 10968 | 1293 | | | | | 461343 | 469645 | |
| 1102 | | | | | | | | | 9785 | 13551 | |
| 1103 | KOC90085.1 | 4404 | 10975 | 1083 | | | | | 30118 | 37991 | |
| 1104 | KOC94081.1 | 4408 | 10979 | 1083 | | | | | 18469 | 26342 | |
| 1105 | KHF78366.1 | 4412 | 10983 | 1069 | | | | | 824611 | 832051 | |
| 1106 | KQB52479.1 | 4416 | 10987 | 1082 | | | | | 8344 | 16187 | |
| 1107 | AJP47986.1 | 4419 | 10990 | 1254 | | | | | 1006536 | 1012681 | |
| 1108 | KIN89384.1 | 4423 | 10994 | 1285 | | | | | 719621 | 727970 | |
| 1109 | ALQ39735.1 | 4427 | 10998 | 1163 | | | | | 878815 | 885968 | |
| 1110 | KIQ83918.1 | 4431 | 11002 | 1078 | | | | | 96014 | 103978 | |
| 1111 | KRP95694.1 | 4435 | 11006 | 1082 | | | | | 120090 | 127912 | |
| 1112 | | | | | | | | | 3688839 | 3693414 | |
| 1113 | | | | | | | | | 318 | 5435 | |
| 1114 | KJM39256.1 | 4445 | 11016 | 1080 | | | | | 95946 | 103809 | |
| 1115 | KJJ99148.1 | 4449 | 11020 | 1260 | | | | | 335865 | 344079 | |
| 1116 | KJQ91386.1 | 4453 | 11024 | 1149 | | | | | 23951 | 31686 | |
| 1117 | KJQ87532.1 | 4457 | 11028 | 1149 | | | | | 23951 | 31686 | |
| 1118 | AKI01766.1 | 4461 | 11032 | 989 | | | | | 3205142 | 3212616 | |
| 1119 | KMP10825.1 | 4464 | 11035 | 1132 | | | | | 4141 | 11432 | |
| 1120 | KJS25179.1 | 4468 | 11039 | 1012 | | | | | 393 | 8801 | |
| 1121 | KJS49373.1 | 4471 | 11042 | 1169 | | | | | 1742 | 9323 | |
| 1122 | KLG11563.1 | 4475 | 11046 | 1079 | | | | | 101392 | 109725 | |
| 1123 | KLP25068.1 | 4479 | 11050 | 1080 | | | | | 12174 | 20036 | |
| 1124 | KLG16382.1 | 4483 | 11054 | 1080 | | | | | 92870 | 100733 | |
| 1125 | AKQ40861.1 | 4487 | 11058 | 1243 | | | | | 103859 | 111640 | |
| 1126 | KLR33908.1 | 4491 | 11062 | 1080 | | | | | 7456 | 15318 | |
| 1127 | KLQ90826.1 | 4495 | 11066 | 1080 | | | | | 420860 | 428722 | |
| 1128 | KYO07693.1 | 4499 | 11070 | 1079 | | | | | 361776 | 370109 | |
| 1129 | KYO16911.1 | 4502 | 11073 | 1080 | | | | | 24041 | 29699 | |
| 1130 | KLP47186.1 | 4506 | 11077 | 1080 | | | | | 39512 | 47374 | |
| 1131 | ALE53301.1 | 4510 | 11081 | 1252 | | | | | 36044 | 44241 | |
| 1132 | KQB93620.1 | 4514 | 11085 | 874 | | | | | 20482 | 26942 | |
| 1133 | AMN49380.1 | 4518 | 11089 | 1075 | | | | | 1279035 | 1286515 | |
| 1134 | | | | | | | | | 3339822 | 3344259 | |
| 1135 | ALK96266.1 | 4525 | 11096 | 1284 | | | | | 1851643 | 1859936 | |
| 1136 | CUJ89784.1 | 4529 | 11100 | 1071 | | | | | 341420 | 348475 | |
| 1137 | CUJ83699.1 | 4533 | 11104 | 1039 | | | | | 311766 | 319390 | |
| 1138 | CUQ65731.1 | 4537 | 11108 | 1151 | | | | | 722567 | 729986 | |
| 1139 | KVV05823.1 | 4541 | 11112 | 1082 | | | | | 20533 | 28381 | |
| 1140 | KVV04343.1 | 4545 | 11116 | 1082 | | | | | 212447 | 220295 | |
| 1141 | KUO77798.1 | 4549 | 11120 | 1163 | | | | | 3229 | 10766 | |
| 1142 | KQM30741.1 | 4553 | 11124 | 1069 | | | | | 115202 | 122116 | |
| 1143 | KQM97535.1 | 4557 | 11128 | 1194 | | | | | 10557 | 18537 | |
| 1144 | KQN80960.1 | 4561 | 11132 | 1069 | | | | | 115205 | 122119 | |
| 1145 | KQQ29439.1 | 4565 | 11136 | 1033 | | | | | 213672 | 221015 | |
| 1146 | KQR49973.1 | 4569 | 11140 | 1250 | | | | | 159043 | 167267 | |
| 1147 | | | | | | | | | 618525 | 622872 | |
| 1148 | KQS63849.1 | 4576 | 11147 | 1033 | | | | | 214815 | 222241 | |
| 1149 | KQV35759.1 | 4580 | 11151 | 1033 | | | | | 269085 | 276511 | |
| 1150 | KQY95045.1 | 4584 | 11155 | 1228 | | | | | 46871 | 54637 | |
| 1151 | KRD25373.1 | 4588 | 11159 | 1093 | | | | | 92331 | 100492 | |
| 1152 | KRD25866.1 | 4592 | 11163 | 1033 | | | | | 269071 | 276497 | |
| 1153 | KQV20176.1 | 4596 | 11167 | 1084 | | | | | 52687 | 60515 | |
| 1154 | KQX34250.1 | 4600 | 11171 | 1028 | | | | | 21428 | 28723 | |
| 1155 | KRE22553.1 | 4604 | 11175 | 999 | | | | | 7778 | 15050 | |
| 1156 | KRA26259.1 | 4608 | 11179 | 1082 | | | | | 142278 | 150100 | |
| 1157 | KRA28531.1 | 4612 | 11183 | 1228 | | | | | 295879 | 303645 | |
| 1158 | KRB49803.1 | 4616 | 11187 | 988 | | | | | 113257 | 120693 | |
| 1159 | KQC46107.1 | 4620 | 11191 | 874 | | | | | 19282 | 25742 | |
| 1160 | KWV49699.1 | 4624 | 11195 | 1032 | | | | | 46982 | 54421 | |
| 1161 | KUI99565.1 | 4628 | 11199 | 1156 | | | | | 1381229 | 1388527 | |
| 1162 | KTG21221.1 | 4632 | 11203 | 1007 | | | | | 2662 | 10353 | |
| 1163 | CVJ90715.1 | 4636 | 11207 | 1285 | | | | | 15727 | 24792 | |
| 1164 | CVK24351.1 | 4640 | 11211 | 1264 | | | | | 173387 | 182161 | |
| 1165 | CVK22607.1 | 4644 | 11215 | 1094 | | | | | 247062 | 254978 | |
| 1166 | CVK27721.1 | 4648 | 11219 | 1104 | | | | | 11000 | 19159 | |
| 1167 | CVJ14724.1 | 4652 | 11223 | 1257 | | | | | 373326 | 382397 | |
| 1168 | CVJ96960.1 | 4656 | 11227 | 1256 | | | | | 4116 | 13150 | |
| 1169 | KXS40894.1 | 4660 | 11231 | 781 | | | | | 1 | 6250 | |
| 1170 | KXW57566.1 | 4664 | 11235 | 1236 | | | | | 203 | 8225 | |
| 1171 | EJO46333.1 | 4668 | 11239 | 1079 | | | | | 3283 | 11091 | |
| 1172 | EPY95206.1 | 4672 | 11243 | 1079 | | | | | 81138 | 89471 | |
| 1173 | EUL64245.1 | 4676 | 11247 | 1079 | | | | | 52392 | 60200 | |
| 1174 | AMQ20315.1 | 4680 | 11251 | 874 | | | | | 858032 | 864492 | |
| 1175 | | | | | ADB42632.1 | 4683 | 11254 | 260 | 34303 | 37171 | |
| 1176 | | | | | EIA72759.1 | 4686 | 11257 | 272 | 4981 | 7798 | |
| 1177 | | | | | EIA87027.1 | 4689 | 11260 | 272 | 587 | 3404 | |
| 1178 | | | | | EIA94949.1 | 4692 | 11263 | 272 | 4929 | 7746 | |
| 1179 | | | | | EIA95447.1 | 4695 | 11266 | 272 | 4929 | 7746 | |
| 1180 | | | | | KQH60906.1 | 4698 | 11269 | 272 | 4977 | 7794 | |
| 1181 | | | | | KQI25490.1 | 4701 | 11272 | 272 | 6992 | 9809 | |
| 1182 | | | | | KQI25382.1 | 4704 | 11275 | 272 | 29293 | 32110 | |
| 1183 | | | | | KRS58893.1 | 4707 | 11278 | 272 | 205116 | 207933 | |
| 1184 | | | | | KRS89291.1 | 4710 | 11281 | 272 | 37988 | 40802 | |
| 1185 | | | | | KEA44660.1 | 4713 | 11284 | 258 | 75386 | 78313 | |
| 1186 | | | | | ALX12000.1 | 4716 | 11287 | 282 | 2362582 | 2365410 | |
| 1187 | | | | | AMB80663.1 | 4719 | 11290 | 171 | 3818332 | 3821205 | |
| 1188 | | | | | CZG58405.1 | 4722 | 11293 | 288 | 228584 | 231634 | |
| 1189 | | | | | CZH63846.1 | 4725 | 11296 | 288 | 299658 | 302708 | |
| 1190 | | | | | CZG92852.1 | 4728 | 11299 | 288 | 132992 | 136042 | |
| 1191 | | | | | CZI24994.1 | 4731 | 11302 | 288 | 5076 | 8126 | |
| 1192 | | | | | CZI29056.1 | 4734 | 11305 | 288 | 236475 | 239525 | |
| 1193 | | | | | CZH75430.1 | 4737 | 11308 | 288 | 4977 | 8027 | |
| 1194 | | | | | CZI28436.1 | 4740 | 11311 | 288 | 236475 | 239525 | |
| 1195 | | | | | CZQ90608.1 | 4743 | 11314 | 288 | 5076 | 8126 | |
| 1196 | | | | | EEZ95916.1 | 4746 | 11317 | 270 | 1176272 | 1179193 | |
| 1197 | | | | | ADG61930.1 | 4749 | 11320 | 238 | 1638409 | 1641357 | |
| 1198 | | | | | EGE12535.1 | 4752 | 11323 | 283 | 142451 | 145399 | |
| 1199 | | | | | EGE10943.1 | 4755 | 11326 | 283 | 32251 | 35199 | |
| 1200 | | | | | EGE11784.1 | 4758 | 11329 | 238 | 35082 | 38030 | |
| 1201 | | | | | EGE15773.1 | 4761 | 11332 | 283 | 36412 | 38991 | |
| 1202 | | | | | EGE17283.1 | 4764 | 11335 | 283 | 104584 | 107532 | |
| 1203 | | | | | EGE18523.1 | 4767 | 11338 | 283 | 113565 | 116513 | |
| 1204 | | | | | EGE26178.1 | 4770 | 11341 | 283 | 113528 | 116215 | |
| 1205 | | | | | EGE24865.1 | 4773 | 11344 | 283 | 113550 | 116498 | |
| 1206 | | | | | EKF83141.1 | 4776 | 11347 | 283 | 182847 | 185795 | |
| 1207 | | | | | AIT43972.1 | 4779 | 11350 | 283 | 1706599 | 1709286 | |
| 1208 | | | | | ALD78150.1 | 4782 | 11353 | 363 | 3528333 | 3531638 | |
| 1209 | | | | | KJI56240.1 | 4785 | 11356 | 363 | 155926 | 159387 | |
| 1210 | | | | | KKW50396.1 | 4788 | 11359 | 286 | 1 | 2089 | |
| 1211 | | | | | KFF70138.1 | 4791 | 11362 | 291 | 156818 | 159810 | |
| 1212 | | | | | KHS98347.1 | 4794 | 11365 | 291 | 92311 | 95303 | |
| 1213 | | | | | AAG59480.1 | 4797 | 11368 | 367 | 5390202 | 5393675 | |
| 1214 | | | | | BAB38679.1 | 4800 | 11371 | 367 | 5360199 | 5363672 | |
| 1215 | | | | | ACA79492.1 | 4803 | 11374 | 367 | 4265907 | 4269380 | |
| 1216 | | | | | ACB17260.1 | 4806 | 11377 | 367 | 4909284 | 4912757 | |
| 1217 | | | | | ACI34823.1 | 4809 | 11380 | 367 | 5433847 | 5437320 | |
| 1218 | | | | | ACT75060.1 | 4812 | 11383 | 367 | 5389908 | 5393381 | |
| 1219 | | | | | ADD59515.1 | 4815 | 11386 | 367 | 5198464 | 5201937 | |
| 1220 | | | | | EEC29617.1 | 4818 | 11389 | 367 | 1434820 | 1438293 | |
| 1221 | | | | | EFK27046.1 | 4821 | 11392 | 367 | 3332 | 6805 | |
| 1222 | | | | | EDU34130.1 | 4824 | 11395 | 367 | 92585 | 96058 | |
| 1223 | | | | | EDU54254.1 | 4827 | 11398 | 367 | 93693 | 97166 | |
| 1224 | | | | | EDU68785.1 | 4830 | 11401 | 367 | 71232 | 74705 | |
| 1225 | | | | | EDU76194.1 | 4833 | 11404 | 367 | 110915 | 114388 | |
| 1226 | | | | | EDU82467.1 | 4836 | 11407 | 367 | 309575 | 313048 | |
| 1227 | | | | | EDU87384.1 | 4839 | 11410 | 367 | 111793 | 115266 | |
| 1228 | | | | | EDU93251.1 | 4842 | 11413 | 367 | 312441 | 315914 | |
| 1229 | | | | | EDU97861.1 | 4845 | 11416 | 367 | 75960 | 79433 | |
| 1230 | | | | | EGI28990.1 | 4848 | 11419 | 367 | 292706 | 296178 | |
| 1231 | | | | | EDZ76317.1 | 4851 | 11422 | 367 | 2709358 | 2712831 | |
| 1232 | | | | | EDZ82369.1 | 4854 | 11425 | 367 | 2577686 | 2581159 | |
| 1233 | | | | | EDZ88717.1 | 4857 | 11428 | 367 | 3697436 | 3700909 | |
| 1234 | | | | | EFX08671.1 | 4860 | 11431 | 367 | 283864 | 287337 | |
| 1235 | | | | | EFX13460.1 | 4863 | 11434 | 367 | 289307 | 292780 | |
| 1236 | | | | | EFX18237.1 | 4866 | 11437 | 367 | 289258 | 292731 | |
| 1237 | | | | | EFX28076.1 | 4869 | 11440 | 367 | 211056 | 214373 | |
| 1238 | | | | | EFX32589.1 | 4872 | 11443 | 367 | 18337 | 21654 | |
| 1239 | | | | | EGD70491.1 | 4875 | 11446 | 367 | 274421 | 277894 | |
| 1240 | | | | | EGD69131.1 | 4878 | 11449 | 367 | 361430 | 364903 | |
| 1241 | | | | | EFW65236.1 | 4881 | 11452 | 367 | 470631 | 474104 | |
| 1242 | | | | | AEZ43445.1 | 4884 | 11455 | 367 | 5125498 | 5128971 | |
| 1243 | | | | | EHU86057.1 | 4887 | 11458 | 367 | 77320 | 80793 | |
| 1244 | | | | | EHU89502.1 | 4890 | 11461 | 367 | 311953 | 315268 | |
| 1245 | | | | | EHV18497.1 | 4893 | 11464 | 367 | 37259 | 40576 | |
| 1246 | | | | | EHV31799.1 | 4896 | 11467 | 367 | 307976 | 311449 | |
| 1247 | | | | | EHV46591.1 | 4899 | 11470 | 367 | 161927 | 165244 | |
| 1248 | | | | | EIF87144.1 | 4902 | 11473 | 367 | 447700 | 451173 | |
| 1249 | | | | | AFJ31991.1 | 4905 | 11476 | 367 | 5247976 | 5251449 | |
| 1250 | | | | | EIN15907.1 | 4908 | 11479 | 367 | 9700 | 13173 | |
| 1251 | | | | | EIP63614.1 | 4911 | 11482 | 367 | 76951 | 80424 | |
| 1252 | | | | | EIP71436.1 | 4914 | 11485 | 367 | 10354 | 13827 | |
| 1253 | | | | | EIO79098.1 | 4917 | 11488 | 351 | 77077 | 80550 | |
| 1254 | | | | | EIN94506.1 | 4920 | 11491 | 367 | 76794 | 80267 | |
| 1255 | | | | | EIO32393.1 | 4923 | 11494 | 367 | 78914 | 82387 | |
| 1256 | | | | | EIO45814.1 | 4926 | 11497 | 367 | 241456 | 244929 | |
| 1257 | | | | | EIO63773.1 | 4929 | 11500 | 367 | 250314 | 253787 | |
| 1258 | | | | | EIO69655.1 | 4932 | 11503 | 367 | 10239 | 13712 | |
| 1259 | | | | | EIO86632.1 | 4935 | 11506 | 367 | 250688 | 254161 | |
| 1260 | | | | | EIP72923.1 | 4938 | 11509 | 367 | 9692 | 13165 | |
| 1261 | | | | | EIP72311.1 | 4941 | 11512 | 367 | 26220 | 29693 | |
| 1262 | | | | | EIP50027.1 | 4944 | 11515 | 367 | 26239 | 29712 | |
| 1263 | | | | | EIP49011.1 | 4947 | 11518 | 367 | 9686 | 13159 | |
| 1264 | | | | | EIP40252.1 | 4950 | 11521 | 367 | 9696 | 13169 | |
| 1265 | | | | | EIP35337.1 | 4953 | 11524 | 367 | 26235 | 29708 | |
| 1266 | | | | | EIP27296.1 | 4956 | 11527 | 367 | 23530 | 26709 | |
| 1267 | | | | | EIP38851.1 | 4959 | 11530 | 367 | 75543 | 79016 | |
| 1268 | | | | | EIP33940.1 | 4962 | 11533 | 367 | 9690 | 13163 | |
| 1269 | | | | | EIP10351.1 | 4965 | 11536 | 367 | 9692 | 13165 | |
| 1270 | | | | | EIP05659.1 | 4968 | 11539 | 367 | 7027 | 10500 | |
| 1271 | | | | | EIP03401.1 | 4971 | 11542 | 367 | 1558 | 4737 | |
| 1272 | | | | | EIO92489.1 | 4974 | 11545 | 367 | 26262 | 29735 | |
| 1273 | | | | | EIO87488.1 | 4977 | 11548 | 367 | 26302 | 29775 | |
| 1274 | | | | | EIN16731.1 | 4980 | 11551 | 367 | 9693 | 13166 | |
| 1275 | | | | | EIN15643.1 | 4983 | 11554 | 367 | 3483 | 6956 | |
| 1276 | | | | | EIN33047.1 | 4986 | 11557 | 367 | 9701 | 13174 | |
| 1277 | | | | | EIO86815.1 | 4989 | 11560 | 39 | 1 | 1412 | |
| 1278 | | | | | EIO65805.1 | 4992 | 11563 | 367 | 9691 | 13164 | |
| 1279 | | | | | EIO79511.1 | 4995 | 11566 | 162 | 3560 | 6418 | |
| 1280 | | | | | EIO49184.1 | 4998 | 11569 | 367 | 9700 | 13173 | |
| 1281 | | | | | EIO29775.1 | 5001 | 11572 | 367 | 1758 | 5231 | |
| 1282 | | | | | EIO24810.1 | 5004 | 11575 | 105 | 9701 | 12947 | |
| 1283 | | | | | EIO11191.1 | 5007 | 11578 | 367 | 9700 | 13173 | |
| 1284 | | | | | EIO07423.1 | 5010 | 11581 | 367 | 9694 | 13167 | |
| 1285 | | | | | EIO07038.1 | 5013 | 11584 | 367 | 9698 | 13171 | |
| 1286 | | | | | EIO09723.1 | 5016 | 11587 | 367 | 9696 | 13169 | |
| 1287 | | | | | EIN89792.1 | 5019 | 11590 | 367 | 112339 | 115812 | |
| 1288 | | | | | EIN88208.1 | 5022 | 11593 | 367 | 26149 | 29622 | |
| 1289 | | | | | EIN85417.1 | 5025 | 11596 | 367 | 9700 | 13173 | |
| 1290 | | | | | EIN69800.1 | 5028 | 11599 | 367 | 9696 | 13169 | |
| 1291 | | | | | EIN65411.1 | 5031 | 11602 | 367 | 9693 | 13166 | |
| 1292 | | | | | EIN68644.1 | 5034 | 11605 | 367 | 9696 | 13169 | |
| 1293 | | | | | EIN52364.1 | 5037 | 11608 | 367 | 9696 | 13169 | |
| 1294 | | | | | EIN49110.1 | 5040 | 11611 | 367 | 26227 | 29700 | |
| 1295 | | | | | EIN33834.1 | 5043 | 11614 | 367 | 9692 | 13165 | |
| 1296 | | | | | EIN35237.1 | 5046 | 11617 | 367 | 29162 | 32635 | |
| 1297 | | | | | EKG95779.1 | 5049 | 11620 | 367 | 3432 | 6905 | |
| 1298 | | | | | EKH07678.1 | 5052 | 11623 | 367 | 364931 | 368404 | |
| 1299 | | | | | EKJ54993.1 | 5055 | 11626 | 367 | 9700 | 13173 | |
| 1300 | | | | | EKJ48079.1 | 5058 | 11629 | 367 | 9705 | 13178 | |
| 1301 | | | | | EKJ52900.1 | 5061 | 11632 | 367 | 9688 | 13161 | |
| 1302 | | | | | EKJ36496.1 | 5064 | 11635 | 367 | 9694 | 13167 | |
| 1303 | | | | | EKJ47660.1 | 5067 | 11638 | 367 | 9700 | 13173 | |
| 1304 | | | | | EKJ36025.1 | 5070 | 11641 | 367 | 9698 | 13171 | |
| 1305 | | | | | EKJ20860.1 | 5073 | 11644 | 367 | 9698 | 13015 | |
| 1306 | | | | | EKJ19822.1 | 5076 | 11647 | 367 | 9694 | 13167 | |
| 1307 | | | | | EKJ00849.1 | 5079 | 11650 | 367 | 23510 | 26983 | |
| 1308 | | | | | EKJ92950.1 | 5082 | 11653 | 367 | 112428 | 115901 | |
| 1309 | | | | | EKJ05366.1 | 5085 | 11656 | 367 | 26146 | 29619 | |
| 1310 | | | | | EKI82673.1 | 5088 | 11659 | 367 | 26272 | 29745 | |
| 1311 | | | | | EKI91868.1 | 5091 | 11662 | 367 | 75553 | 79026 | |
| 1312 | | | | | EKI72761.1 | 5094 | 11665 | 351 | 26220 | 29693 | |
| 1313 | | | | | EKI64836.1 | 5097 | 11668 | 367 | 26230 | 29703 | |
| 1314 | | | | | EKI59121.1 | 5100 | 11671 | 367 | 9693 | 13166 | |
| 1315 | | | | | EKI57124.1 | 5103 | 11674 | 351 | 9695 | 13168 | |
| 1316 | | | | | EKI46664.1 | 5106 | 11677 | 351 | 9693 | 13166 | |
| 1317 | | | | | EKI33384.1 | 5109 | 11680 | 367 | 9694 | 13011 | |
| 1318 | | | | | EKH20915.1 | 5112 | 11683 | 367 | 9699 | 13172 | |
| 1319 | | | | | EKH25284.1 | 5115 | 11686 | 367 | 328332 | 331805 | |
| 1320 | | | | | EKH13350.1 | 5118 | 11689 | 367 | 9702 | 13175 | |
| 1321 | | | | | EKH36313.1 | 5121 | 11692 | 367 | 9704 | 13177 | |
| 1322 | | | | | EKH26861.1 | 5124 | 11695 | 367 | 115401 | 118874 | |
| 1323 | | | | | EKH48827.1 | 5127 | 11698 | 367 | 9700 | 13173 | |
| 1324 | | | | | EKH53676.1 | 5130 | 11701 | 367 | 115476 | 118949 | |
| 1325 | | | | | EKH61521.1 | 5133 | 11704 | 367 | 9699 | 13172 | |
| 1326 | | | | | EKH63163.1 | 5136 | 11707 | 367 | 248863 | 252336 | |
| 1327 | | | | | EKH71773.1 | 5139 | 11710 | 367 | 9699 | 13172 | |
| 1328 | | | | | EKH63846.1 | 5142 | 11713 | 367 | 9702 | 13175 | |
| 1329 | | | | | EKH83482.1 | 5145 | 11716 | 367 | 9704 | 13177 | |
| 1330 | | | | | EKH87898.1 | 5148 | 11719 | 367 | 9702 | 13175 | |
| 1331 | | | | | EKH95563.1 | 5151 | 11722 | 367 | 9704 | 13177 | |
| 1332 | | | | | EKI01260.1 | 5154 | 11725 | 367 | 115400 | 118873 | |
| 1333 | | | | | EKI02729.1 | 5157 | 11728 | 367 | 67397 | 70870 | |
| 1334 | | | | | EKH96736.1 | 5160 | 11731 | 367 | 9432 | 12905 | |
| 1335 | | | | | EKI03038.1 | 5163 | 11734 | 367 | 9488 | 12961 | |
| 1336 | | | | | EKI06057.1 | 5166 | 11737 | 367 | 66831 | 70304 | |
| 1337 | | | | | EKK37392.1 | 5169 | 11740 | 367 | 9688 | 13161 | |
| 1338 | | | | | EKK71764.1 | 5172 | 11743 | 367 | 68104 | 71577 | |
| 1339 | | | | | EKK91181.1 | 5175 | 11746 | 367 | 6240 | 9713 | |
| 1340 | | | | | EKK36880.1 | 5178 | 11749 | 367 | 9701 | 13174 | |
| 1341 | | | | | EKK37975.1 | 5181 | 11752 | 351 | 9698 | 13171 | |
| 1342 | | | | | EKK61087.1 | 5184 | 11755 | 367 | 9682 | 13155 | |
| 1343 | | | | | EKK52330.1 | 5187 | 11758 | 367 | 9684 | 13157 | |
| 1344 | | | | | EKK67937.1 | 5190 | 11761 | 367 | 75545 | 79018 | |
| 1345 | | | | | EKK62289.1 | 5193 | 11764 | 367 | 75520 | 78993 | |
| 1346 | | | | | EKK87665.1 | 5196 | 11767 | 367 | 77628 | 81101 | |
| 1347 | | | | | EKV88763.1 | 5199 | 11770 | 367 | 3477 | 6950 | |
| 1348 | | | | | EKV85096.1 | 5202 | 11773 | 367 | 9696 | 13169 | |
| 1349 | | | | | EKV70650.1 | 5205 | 11776 | 367 | 9694 | 13167 | |
| 1350 | | | | | EKW05769.1 | 5208 | 11779 | 367 | 75769 | 79242 | |
| 1351 | | | | | EKV91511.1 | 5211 | 11782 | 367 | 3479 | 6952 | |
| 1352 | | | | | EKW03278.1 | 5214 | 11785 | 367 | 9690 | 13163 | |
| 1353 | | | | | EKW04231.1 | 5217 | 11788 | 367 | 9698 | 13171 | |
| 1354 | | | | | EKW19022.1 | 5220 | 11791 | 367 | 9700 | 13173 | |
| 1355 | | | | | EKW08485.1 | 5223 | 11794 | 367 | 9700 | 13173 | |
| 1356 | | | | | EKW71369.1 | 5226 | 11797 | 351 | 6744 | 10217 | |
| 1357 | | | | | EKW96653.1 | 5229 | 11800 | 367 | 75179 | 78652 | |
| 1358 | | | | | EKW96258.1 | 5232 | 11803 | 367 | 9885 | 13358 | |
| 1359 | | | | | EKW76354.1 | 5235 | 11806 | 367 | 29183 | 32500 | |
| 1360 | | | | | EKW75840.1 | 5238 | 11809 | 367 | 9851 | 13324 | |
| 1361 | | | | | EKW68010.1 | 5241 | 11812 | 367 | 9909 | 13382 | |
| 1362 | | | | | EKW74813.1 | 5244 | 11815 | 351 | 9682 | 13155 | |
| 1363 | | | | | EKW43904.1 | 5247 | 11818 | 367 | 79656 | 83129 | |
| 1364 | | | | | EKW70673.1 | 5250 | 11821 | 367 | 75702 | 79175 | |
| 1365 | | | | | EKW38122.1 | 5253 | 11824 | 367 | 9692 | 13165 | |
| 1366 | | | | | EKW21069.1 | 5256 | 11827 | 367 | 3479 | 6358 | |
| 1367 | | | | | EKW36076.1 | 5259 | 11830 | 105 | 26295 | 29801 | |
| 1368 | | | | | EKW36716.1 | 5262 | 11833 | 351 | 9702 | 13175 | |
| 1369 | | | | | EKW51351.1 | 5265 | 11836 | 367 | 9684 | 13157 | |
| 1370 | | | | | EKY34589.1 | 5268 | 11839 | 367 | 9698 | 13171 | |
| 1371 | | | | | EKY34727.1 | 5271 | 11842 | 367 | 9696 | 13013 | |
| 1372 | | | | | ELI49198.1 | 5274 | 11845 | 367 | 723 | 4196 | |
| 1373 | | | | | ELV23652.1 | 5277 | 11848 | 367 | 19030 | 22503 | |
| 1374 | | | | | ELV30874.1 | 5280 | 11851 | 367 | 3059 | 6532 | |
| 1375 | | | | | ELV62889.1 | 5283 | 11854 | 367 | 29755 | 33072 | |
| 1376 | | | | | ELV61720.1 | 5286 | 11857 | 367 | 352 | 3504 | |
| 1377 | | | | | ELV63257.1 | 5289 | 11860 | 367 | 482 | 3955 | |
| 1378 | | | | | ELV91640.1 | 5292 | 11863 | 367 | 26114 | 29587 | |
| 1379 | | | | | ELV93147.1 | 5295 | 11866 | 367 | 2876 | 6349 | |
| 1380 | | | | | ELW21803.1 | 5298 | 11869 | 367 | 19055 | 22528 | |
| 1381 | | | | | ELW24260.1 | 5301 | 11872 | 367 | 355 | 3828 | |
| 1382 | | | | | ELW27705.1 | 5304 | 11875 | 367 | 9838 | 13311 | |
| 1383 | | | | | ELW23229.1 | 5307 | 11878 | 367 | 342 | 3815 | |
| 1384 | | | | | ELW28314.1 | 5310 | 11881 | 367 | 9868 | 13341 | |
| 1385 | | | | | ELW46368.1 | 5313 | 11884 | 367 | 9890 | 13363 | |
| 1386 | | | | | ELV14531.1 | 5316 | 11887 | 367 | 362 | 3835 | |
| 1387 | | | | | ELV31269.1 | 5319 | 11890 | 367 | 26209 | 29682 | |
| 1388 | | | | | ELV35520.1 | 5322 | 11893 | 367 | 26046 | 29519 | |
| 1389 | | | | | ELV44401.1 | 5325 | 11896 | 367 | 1488 | 4667 | |
| 1390 | | | | | ELV47161.1 | 5328 | 11899 | 367 | 9825 | 13298 | |
| 1391 | | | | | ELV76551.1 | 5331 | 11902 | 367 | 18805 | 22278 | |
| 1392 | | | | | ELV98803.1 | 5334 | 11905 | 367 | 9948 | 13421 | |
| 1393 | | | | | ELV75965.1 | 5337 | 11908 | 367 | 9795 | 13268 | |
| 1394 | | | | | ELV90960.1 | 5340 | 11911 | 367 | 351 | 3824 | |
| 1395 | | | | | ELW12422.1 | 5343 | 11914 | 367 | 9877 | 13350 | |
| 1396 | | | | | ELV15306.1 | 5346 | 11917 | 351 | 9689 | 13162 | |
| 1397 | | | | | EMR91944.1 | 5349 | 11920 | 367 | 5144 | 8617 | |
| 1398 | | | | | EMS03598.1 | 5352 | 11923 | 367 | 7561 | 11034 | |
| 1399 | | | | | ELE41778.1 | 5355 | 11926 | 365 | 3624619 | 3628086 | |
| 1400 | | | | | ELF28430.1 | 5358 | 11929 | 367 | 2021904 | 2025377 | |
| 1401 | | | | | ELG08153.1 | 5361 | 11932 | 367 | 1531417 | 1534890 | |
| 1402 | | | | | ELH63188.1 | 5364 | 11935 | 367 | 388742 | 392215 | |
| 1403 | | | | | ENE27217.1 | 5367 | 11938 | 172 | 180 | 3065 | |
| 1404 | | | | | EQO57390.1 | 5370 | 11941 | 367 | 620369 | 623842 | |
| 1405 | | | | | EQO66401.1 | 5373 | 11944 | 367 | 1177818 | 1181291 | |
| 1406 | | | | | EQT31737.1 | 5376 | 11947 | 367 | 348841 | 352314 | |
| 1407 | | | | | EQT84556.1 | 5379 | 11950 | 367 | 632377 | 635850 | |
| 1408 | | | | | ERB08802.1 | 5382 | 11953 | 367 | 3407072 | 3410545 | |
| 1409 | | | | | ERB13354.1 | 5385 | 11956 | 367 | 322637 | 326110 | |
| 1410 | | | | | ERC00100.1 | 5388 | 11959 | 367 | 77309 | 80782 | |
| 1411 | | | | | ERB68139.1 | 5391 | 11962 | 367 | 75975 | 79448 | |
| 1412 | | | | | ERB88971.1 | 5394 | 11965 | 367 | 9811 | 13284 | |
| 1413 | | | | | ERC15172.1 | 5397 | 11968 | 367 | 9856 | 13329 | |
| 1414 | | | | | ERB79213.1 | 5400 | 11971 | 367 | 77187 | 80660 | |
| 1415 | | | | | ERB91730.1 | 5403 | 11974 | 367 | 77192 | 80665 | |
| 1416 | | | | | ERC08309.1 | 5406 | 11977 | 367 | 9951 | 13424 | |
| 1417 | | | | | ERC32303.1 | 5409 | 11980 | 367 | 9828 | 13301 | |
| 1418 | | | | | ERC25307.1 | 5412 | 11983 | 367 | 249232 | 252705 | |
| 1419 | | | | | ERC09301.1 | 5415 | 11986 | 367 | 9873 | 13346 | |
| 1420 | | | | | ERC10656.1 | 5418 | 11989 | 367 | 9861 | 13334 | |
| 1421 | | | | | ERE14325.1 | 5421 | 11992 | 367 | 78160 | 81633 | |
| 1422 | | | | | ERC92365.1 | 5424 | 11995 | 367 | 503 | 3976 | |
| 1423 | | | | | ERD40228.1 | 5427 | 11998 | 367 | 9979 | 13452 | |
| 1424 | | | | | ERC91693.1 | 5430 | 12001 | 367 | 78422 | 81895 | |
| 1425 | | | | | ERD09262.1 | 5433 | 12004 | 367 | 9938 | 13411 | |
| 1426 | | | | | ERC65820.1 | 5436 | 12007 | 367 | 76813 | 80286 | |
| 1427 | | | | | ERC53515.1 | 5439 | 12010 | 367 | 79318 | 82791 | |
| 1428 | | | | | ERE00879.1 | 5442 | 12013 | 367 | 79822 | 83295 | |
| 1429 | | | | | ERE21794.1 | 5445 | 12016 | 367 | 77012 | 80485 | |
| 1430 | | | | | ERC61432.1 | 5448 | 12019 | 367 | 29621 | 33094 | |
| 1431 | | | | | ERC74183.1 | 5451 | 12022 | 367 | 7928 | 11401 | |
| 1432 | | | | | ERC30058.1 | 5454 | 12025 | 367 | 249421 | 252894 | |
| 1433 | | | | | ERC33931.1 | 5457 | 12028 | 367 | 251930 | 255403 | |
| 1434 | | | | | ERC40338.1 | 5460 | 12031 | 367 | 248447 | 251920 | |
| 1435 | | | | | ERC48255.1 | 5463 | 12034 | 367 | 313847 | 317320 | |
| 1436 | | | | | ERD08377.1 | 5466 | 12037 | 367 | 10188 | 13661 | |
| 1437 | | | | | ERD05017.1 | 5469 | 12040 | 367 | 10008 | 13481 | |
| 1438 | | | | | ERD20744.1 | 5472 | 12043 | 367 | 10127 | 13600 | |
| 1439 | | | | | ERD22948.1 | 5475 | 12046 | 367 | 9957 | 13430 | |
| 1440 | | | | | ERD23988.1 | 5478 | 12049 | 367 | 9873 | 13346 | |
| 1441 | | | | | ERD35067.1 | 5481 | 12052 | 367 | 10031 | 13504 | |
| 1442 | | | | | ERD37181.1 | 5484 | 12055 | 367 | 9911 | 13384 | |
| 1443 | | | | | ERD26789.1 | 5487 | 12058 | 367 | 29789 | 33106 | |
| 1444 | | | | | ERD52927.1 | 5490 | 12061 | 367 | 9940 | 13413 | |
| 1445 | | | | | ERD45125.1 | 5493 | 12064 | 367 | 9940 | 13413 | |
| 1446 | | | | | ERD72595.1 | 5496 | 12067 | 367 | 9928 | 13401 | |
| 1447 | | | | | ERD68374.1 | 5499 | 12070 | 367 | 9876 | 13349 | |
| 1448 | | | | | ERD73628.1 | 5502 | 12073 | 367 | 9971 | 13288 | |
| 1449 | | | | | ERD83185.1 | 5505 | 12076 | 367 | 9864 | 13337 | |
| 1450 | | | | | ERD87988.1 | 5508 | 12079 | 367 | 9921 | 13394 | |
| 1451 | | | | | ERD91773.1 | 5511 | 12082 | 367 | 10046 | 13519 | |
| 1452 | | | | | ERD98680.1 | 5514 | 12085 | 367 | 9963 | 13436 | |
| 1453 | | | | | ERD88425.1 | 5517 | 12088 | 367 | 9908 | 13225 | |
| 1454 | | | | | ERE36108.1 | 5520 | 12091 | 367 | 9963 | 13436 | |
| 1455 | | | | | ERE23637.1 | 5523 | 12094 | 367 | 324 | 3797 | |
| 1456 | | | | | ERE42058.1 | 5526 | 12097 | 367 | 76380 | 79853 | |
| 1457 | | | | | ERE45406.1 | 5529 | 12100 | 367 | 76828 | 80301 | |
| 1458 | | | | | ETJ70766.1 | 5532 | 12103 | 367 | 294 | 3767 | |
| 1459 | | | | | ERC07678.1 | 5535 | 12106 | 367 | 9843 | 13316 | |
| 1460 | | | | | ESD10537.1 | 5538 | 12109 | 367 | 455 | 3928 | |
| 1461 | | | | | ESL20618.1 | 5541 | 12112 | 367 | 2605262 | 2608735 | |
| 1462 | | | | | ESP12224.1 | 5544 | 12115 | 367 | 347406 | 350879 | |
| 1463 | | | | | ETJ79051.1 | 5547 | 12118 | 367 | 9638 | 13111 | |
| 1464 | | | | | EST67464.1 | 5550 | 12121 | 367 | 62231 | 65703 | |
| 1465 | | | | | EST67378.1 | 5553 | 12124 | 367 | 35969 | 39442 | |
| 1466 | | | | | AHG12153.1 | 5556 | 12127 | 367 | 5404978 | 5408295 | |
| 1467 | | | | | EYE30765.1 | 5559 | 12130 | 367 | 13431 | 16904 | |
| 1468 | | | | | EYE15507.1 | 5562 | 12133 | 367 | 8798 | 12271 | |
| 1469 | | | | | EYE15594.1 | 5565 | 12136 | 367 | 8718 | 12191 | |
| 1470 | | | | | EYW09909.1 | 5568 | 12139 | 367 | 361634 | 365107 | |
| 1471 | | | | | EYW25816.1 | 5571 | 12142 | 367 | 361618 | 365091 | |
| 1472 | | | | | EYW27178.1 | 5574 | 12145 | 367 | 75562 | 79035 | |
| 1473 | | | | | EYW07429.1 | 5577 | 12148 | 367 | 9641 | 13114 | |
| 1474 | | | | | EYW24507.1 | 5580 | 12151 | 367 | 76003 | 79476 | |
| 1475 | | | | | EYW34607.1 | 5583 | 12154 | 367 | 9641 | 13114 | |
| 1476 | | | | | EYW13498.1 | 5586 | 12157 | 367 | 76031 | 79504 | |
| 1477 | | | | | EYV89289.1 | 5589 | 12160 | 367 | 361672 | 365145 | |
| 1478 | | | | | EYY46581.1 | 5592 | 12163 | 367 | 358618 | 362091 | |
| 1479 | | | | | EYV70452.1 | 5595 | 12166 | 367 | 125628 | 129101 | |
| 1480 | | | | | EYV67644.1 | 5598 | 12169 | 367 | 298415 | 301888 | |
| 1481 | | | | | EYV59033.1 | 5601 | 12172 | 367 | 136307 | 139780 | |
| 1482 | | | | | EYV37956.1 | 5604 | 12175 | 367 | 6474 | 9947 | |
| 1483 | | | | | EYV46021.1 | 5607 | 12178 | 367 | 116589 | 120062 | |
| 1484 | | | | | EYV44254.1 | 5610 | 12181 | 367 | 135140 | 138613 | |
| 1485 | | | | | EYV24871.1 | 5613 | 12184 | 367 | 136213 | 139686 | |
| 1486 | | | | | EYV22947.1 | 5616 | 12187 | 367 | 18805 | 22278 | |
| 1487 | | | | | EYV18172.1 | 5619 | 12190 | 367 | 116382 | 119855 | |
| 1488 | | | | | EYV20082.1 | 5622 | 12193 | 367 | 17020 | 20493 | |
| 1489 | | | | | EYV16480.1 | 5625 | 12196 | 367 | 135221 | 138694 | |
| 1490 | | | | | EYW83369.1 | 5628 | 12199 | 367 | 361672 | 365145 | |
| 1491 | | | | | EYW75517.1 | 5631 | 12202 | 367 | 9640 | 13113 | |
| 1492 | | | | | EYW71147.1 | 5634 | 12205 | 367 | 9640 | 13113 | |
| 1493 | | | | | EYW67183.1 | 5637 | 12208 | 367 | 361537 | 365010 | |
| 1494 | | | | | EYW64052.1 | 5640 | 12211 | 367 | 361536 | 365009 | |
| 1495 | | | | | EYW57180.1 | 5643 | 12214 | 367 | 361537 | 365010 | |
| 1496 | | | | | EYW49138.1 | 5646 | 12217 | 367 | 9640 | 13113 | |
| 1497 | | | | | EYW49451.1 | 5649 | 12220 | 367 | 9640 | 13113 | |
| 1498 | | | | | EYW43052.1 | 5652 | 12223 | 367 | 361537 | 365010 | |
| 1499 | | | | | EYW39158.1 | 5655 | 12226 | 367 | 9640 | 13113 | |
| 1500 | | | | | EYX70056.1 | 5658 | 12229 | 367 | 246056 | 249529 | |
| 1501 | | | | | EYX57447.1 | 5661 | 12232 | 367 | 361672 | 365145 | |
| 1502 | | | | | EYX54552.1 | 5664 | 12235 | 367 | 361672 | 365145 | |
| 1503 | | | | | EYX48138.1 | 5667 | 12238 | 367 | 361672 | 365145 | |
| 1504 | | | | | EYX44885.1 | 5670 | 12241 | 367 | 361672 | 365145 | |
| 1505 | | | | | EYX43989.1 | 5673 | 12244 | 367 | 9641 | 13114 | |
| 1506 | | | | | EYX33008.1 | 5676 | 12247 | 367 | 361672 | 365145 | |
| 1507 | | | | | EYX30700.1 | 5679 | 12250 | 367 | 361672 | 365145 | |
| 1508 | | | | | EYX27757.1 | 5682 | 12253 | 367 | 9641 | 13114 | |
| 1509 | | | | | EYX13624.1 | 5685 | 12256 | 367 | 9642 | 13115 | |
| 1510 | | | | | EYX12331.1 | 5688 | 12259 | 367 | 361671 | 365144 | |
| 1511 | | | | | EYX02200.1 | 5691 | 12262 | 367 | 9639 | 13112 | |
| 1512 | | | | | EYW98548.1 | 5694 | 12265 | 367 | 135915 | 139388 | |
| 1513 | | | | | EYW85336.1 | 5697 | 12268 | 367 | 361672 | 365145 | |
| 1514 | | | | | EYV84727.1 | 5700 | 12271 | 367 | 361904 | 365377 | |
| 1515 | | | | | EYZ76666.1 | 5703 | 12274 | 367 | 114299 | 117772 | |
| 1516 | | | | | EYZ63884.1 | 5706 | 12277 | 367 | 22138 | 25611 | |
| 1517 | | | | | EYZ52262.1 | 5709 | 12280 | 367 | 361630 | 365103 | |
| 1518 | | | | | EYZ37412.1 | 5712 | 12283 | 367 | 9641 | 13114 | |
| 1519 | | | | | EYZ38311.1 | 5715 | 12286 | 367 | 361657 | 365130 | |
| 1520 | | | | | EYZ36221.1 | 5718 | 12289 | 367 | 361632 | 365105 | |
| 1521 | | | | | EYZ14533.1 | 5721 | 12292 | 367 | 16533 | 20006 | |
| 1522 | | | | | EZC28520.1 | 5724 | 12295 | 367 | 75292 | 78765 | |
| 1523 | | | | | EZB48634.1 | 5727 | 12298 | 367 | 10661 | 14134 | |
| 1524 | | | | | EZB05978.1 | 5730 | 12301 | 367 | 361672 | 365145 | |
| 1525 | | | | | EZB18139.1 | 5733 | 12304 | 367 | 75494 | 78967 | |
| 1526 | | | | | EZA89755.1 | 5736 | 12307 | 367 | 361630 | 365103 | |
| 1527 | | | | | EZB32011.1 | 5739 | 12310 | 367 | 9642 | 13115 | |
| 1528 | | | | | EZB20624.1 | 5742 | 12313 | 367 | 361672 | 365145 | |
| 1529 | | | | | EZB41520.1 | 5745 | 12316 | 367 | 9639 | 13112 | |
| 1530 | | | | | EZB11569.1 | 5748 | 12319 | 367 | 361763 | 365236 | |
| 1531 | | | | | EZB46981.1 | 5751 | 12322 | 367 | 9639 | 13112 | |
| 1532 | | | | | EZB04407.1 | 5754 | 12325 | 367 | 361672 | 365145 | |
| 1533 | | | | | EZB74686.1 | 5757 | 12328 | 367 | 9945 | 13418 | |
| 1534 | | | | | EZB73670.1 | 5760 | 12331 | 367 | 9018 | 12491 | |
| 1535 | | | | | EZB73226.1 | 5763 | 12334 | 367 | 1813 | 5286 | |
| 1536 | | | | | EZB87907.1 | 5766 | 12337 | 367 | 10203 | 13676 | |
| 1537 | | | | | EZB80139.1 | 5769 | 12340 | 367 | 361406 | 364879 | |
| 1538 | | | | | EZB81691.1 | 5772 | 12343 | 367 | 9836 | 13309 | |
| 1539 | | | | | EZB96174.1 | 5775 | 12346 | 367 | 1 | 2617 | |
| 1540 | | | | | EZC15363.1 | 5778 | 12349 | 367 | 10311 | 13628 | |
| 1541 | | | | | EZC19639.1 | 5781 | 12352 | 367 | 10014 | 13487 | |
| 1542 | | | | | EZC20552.1 | 5784 | 12355 | 367 | 9755 | 13228 | |
| 1543 | | | | | EZC30168.1 | 5787 | 12358 | 367 | 363212 | 366685 | |
| 1544 | | | | | EZC37649.1 | 5790 | 12361 | 367 | 361839 | 365312 | |
| 1545 | | | | | EZC43979.1 | 5793 | 12364 | 367 | 247561 | 251034 | |
| 1546 | | | | | EZB95446.1 | 5796 | 12367 | 367 | 156780 | 160253 | |
| 1547 | | | | | EZC48752.1 | 5799 | 12370 | 367 | 361737 | 365210 | |
| 1548 | | | | | EZC62959.1 | 5802 | 12373 | 367 | 73369 | 76842 | |
| 1549 | | | | | EZC70418.1 | 5805 | 12376 | 367 | 10529 | 14002 | |
| 1550 | | | | | EZC77658.1 | 5808 | 12379 | 367 | 9929 | 13402 | |
| 1551 | | | | | EZC64799.1 | 5811 | 12382 | 367 | 361985 | 365458 | |
| 1552 | | | | | EZC90329.1 | 5814 | 12385 | 367 | 9858 | 13331 | |
| 1553 | | | | | EZC92767.1 | 5817 | 12388 | 367 | 10327 | 13800 | |
| 1554 | | | | | EZC86949.1 | 5820 | 12391 | 367 | 9288 | 12761 | |
| 1555 | | | | | EZC83410.1 | 5823 | 12394 | 367 | 10360 | 13833 | |
| 1556 | | | | | EZD75265.1 | 5826 | 12397 | 367 | 9641 | 13114 | |
| 1557 | | | | | EZF08564.1 | 5829 | 12400 | 367 | 361634 | 365107 | |
| 1558 | | | | | EZF05485.1 | 5832 | 12403 | 367 | 361566 | 365039 | |
| 1559 | | | | | EZE61417.1 | 5835 | 12406 | 367 | 79039 | 82512 | |
| 1560 | | | | | EZE88802.1 | 5838 | 12409 | 367 | 10505 | 13978 | |
| 1561 | | | | | EZE89445.1 | 5841 | 12412 | 367 | 247303 | 250776 | |
| 1562 | | | | | EZE90571.1 | 5844 | 12415 | 367 | 17451 | 20924 | |
| 1563 | | | | | EZD08972.1 | 5847 | 12418 | 367 | 361866 | 365339 | |
| 1564 | | | | | EZD16777.1 | 5850 | 12421 | 367 | 298547 | 301798 | |
| 1565 | | | | | EZD15761.1 | 5853 | 12424 | 367 | 361275 | 364748 | |
| 1566 | | | | | EZD19492.1 | 5856 | 12427 | 367 | 361814 | 365287 | |
| 1567 | | | | | EZD00271.1 | 5859 | 12430 | 367 | 91957 | 95430 | |
| 1568 | | | | | EZD85326.1 | 5862 | 12433 | 367 | 76047 | 79520 | |
| 1569 | | | | | EZD90120.1 | 5865 | 12436 | 367 | 9641 | 13114 | |
| 1570 | | | | | EZJ86913.1 | 5868 | 12439 | 367 | 311 | 3784 | |
| 1571 | | | | | EZJ76225.1 | 5871 | 12442 | 367 | 2720 | 6037 | |
| 1572 | | | | | EZJ72756.1 | 5874 | 12445 | 367 | 645 | 4118 | |
| 1573 | | | | | EZK00743.1 | 5877 | 12448 | 367 | 2714 | 6187 | |
| 1574 | | | | | EZQ47390.1 | 5880 | 12451 | 367 | 298328 | 301801 | |
| 1575 | | | | | EZQ50286.1 | 5883 | 12454 | 367 | 361835 | 365308 | |
| 1576 | | | | | AHY73972.1 | 5886 | 12457 | 367 | 5404976 | 5408293 | |
| 1577 | | | | | KDA71003.1 | 5889 | 12460 | 367 | 9097 | 12570 | |
| 1578 | | | | | KDA57922.1 | 5892 | 12463 | 365 | 4997 | 8308 | |
| 1579 | | | | | KDM81017.1 | 5895 | 12466 | 367 | 152275 | 155748 | |
| 1580 | | | | | KDM84276.1 | 5898 | 12469 | 367 | 16699 | 20172 | |
| 1581 | | | | | KDT83301.1 | 5901 | 12472 | 367 | 26840 | 30313 | |
| 1582 | | | | | KDX42981.1 | 5904 | 12475 | 365 | 42388 | 45699 | |
| 1583 | | | | | KEJ21095.1 | 5907 | 12478 | 367 | 8693 | 12166 | |
| 1584 | | | | | KEJ20439.1 | 5910 | 12481 | 367 | 8678 | 12151 | |
| 1585 | | | | | KEM49613.1 | 5913 | 12484 | 367 | 27031 | 30504 | |
| 1586 | | | | | KEM85728.1 | 5916 | 12487 | 367 | 26649 | 30122 | |
| 1587 | | | | | KEN65094.1 | 5919 | 12490 | 367 | 726 | 4199 | |
| 1588 | | | | | KEO16488.1 | 5922 | 12493 | 365 | 5063 | 8374 | |
| 1589 | | | | | KEO21425.1 | 5925 | 12496 | 367 | 264 | 3737 | |
| 1590 | | | | | KEK80774.1 | 5928 | 12499 | 367 | 61240 | 64713 | |
| 1591 | | | | | KEK94611.1 | 5931 | 12502 | 367 | 26970 | 30443 | |
| 1592 | | | | | KEL07349.1 | 5934 | 12505 | 367 | 24905 | 28378 | |
| 1593 | | | | | KEL97935.1 | 5937 | 12508 | 367 | 1475 | 4948 | |
| 1594 | | | | | KEM08523.1 | 5940 | 12511 | 367 | 26927 | 30400 | |
| 1595 | | | | | AIF96930.1 | 5943 | 12514 | 367 | 5385621 | 5389094 | |
| 1596 | | | | | AIG71770.1 | 5946 | 12517 | 367 | 5409083 | 5412556 | |
| 1597 | | | | | KHI41606.1 | 5949 | 12520 | 367 | 168373 | 171846 | |
| 1598 | | | | | KHH50141.1 | 5952 | 12523 | 365 | 52610 | 56077 | |
| 1599 | | | | | KHI22223.1 | 5955 | 12526 | 367 | 1989 | 5462 | |
| 1600 | | | | | AJA29432.1 | 5958 | 12529 | 367 | 5350388 | 5353861 | |
| 1601 | | | | | KIG29574.1 | 5961 | 12532 | 367 | 33089 | 36562 | |
| 1602 | | | | | KIY28269.1 | 5964 | 12535 | 367 | 10372 | 13845 | |
| 1603 | | | | | KIZ09775.1 | 5967 | 12538 | 367 | 9762 | 13235 | |
| 1604 | | | | | KJJ46590.1 | 5970 | 12541 | 367 | 66829 | 70302 | |
| 1605 | | | | | KJW55351.1 | 5973 | 12544 | 367 | 136995 | 140468 | |
| 1606 | | | | | KJW51699.1 | 5976 | 12547 | 367 | 116958 | 120431 | |
| 1607 | | | | | KKF77969.1 | 5979 | 12550 | 367 | 10494 | 13967 | |
| 1608 | | | | | KKF80394.1 | 5982 | 12553 | 367 | 9762 | 13235 | |
| 1609 | | | | | KKK30169.1 | 5985 | 12556 | 367 | 2135963 | 2139436 | |
| 1610 | | | | | KKY44748.1 | 5988 | 12559 | 149 | 1 | 2817 | |
| 1611 | | | | | KLG52814.1 | 5991 | 12562 | 367 | 114307 | 117780 | |
| 1612 | | | | | KNF16833.1 | 5994 | 12565 | 367 | 1989 | 5462 | |
| 1613 | | | | | KNF20998.1 | 5997 | 12568 | 367 | 114447 | 117920 | |
| 1614 | | | | | KNF39643.1 | 6000 | 12571 | 367 | 16655 | 20128 | |
| 1615 | | | | | KNG40955.1 | 6003 | 12574 | 367 | 146674 | 150147 | |
| 1616 | | | | | CTT73476.1 | 6006 | 12577 | 367 | 5649 | 9122 | |
| 1617 | | | | | KOZ12762.1 | 6009 | 12580 | 367 | 78490 | 81963 | |
| 1618 | | | | | KOZ62644.1 | 6012 | 12583 | 367 | 9646 | 13119 | |
| 1619 | | | | | KOZ92034.1 | 6015 | 12586 | 367 | 47766 | 51239 | |
| 1620 | | | | | KOZ11133.1 | 6018 | 12589 | 367 | 9649 | 13122 | |
| 1621 | | | | | KOZ14563.1 | 6021 | 12592 | 367 | 248571 | 252044 | |
| 1622 | | | | | KOZ32806.1 | 6024 | 12595 | 367 | 75809 | 79282 | |
| 1623 | | | | | KOZ23194.1 | 6027 | 12598 | 367 | 361705 | 365178 | |
| 1624 | | | | | KOZ33144.1 | 6030 | 12601 | 367 | 9646 | 13119 | |
| 1625 | | | | | KOZ37733.1 | 6033 | 12604 | 367 | 9647 | 13120 | |
| 1626 | | | | | KOZ50075.1 | 6036 | 12607 | 367 | 361612 | 365085 | |
| 1627 | | | | | KOZ47192.1 | 6039 | 12610 | 367 | 9644 | 13117 | |
| 1628 | | | | | KOZ49648.1 | 6042 | 12613 | 367 | 361767 | 365240 | |
| 1629 | | | | | KOZ66198.1 | 6045 | 12616 | 367 | 67507 | 70980 | |
| 1630 | | | | | KOZ69679.1 | 6048 | 12619 | 367 | 361543 | 365016 | |
| 1631 | | | | | KOZ74920.1 | 6051 | 12622 | 367 | 17746 | 21219 | |
| 1632 | | | | | KOZ84732.1 | 6054 | 12625 | 367 | 66782 | 70255 | |
| 1633 | | | | | KOZ80207.1 | 6057 | 12628 | 367 | 66781 | 70254 | |
| 1634 | | | | | KOZ97680.1 | 6060 | 12631 | 367 | 150289 | 153762 | |
| 1635 | | | | | KPH45896.1 | 6063 | 12634 | 367 | 248360 | 251833 | |
| 1636 | | | | | KPH35133.1 | 6066 | 12637 | 367 | 361701 | 365174 | |
| 1637 | | | | | KPH38254.1 | 6069 | 12640 | 367 | 78493 | 81966 | |
| 1638 | | | | | ALH93592.1 | 6072 | 12643 | 367 | 5193818 | 5197291 | |
| 1639 | | | | | KPO74167.1 | 6075 | 12646 | 367 | 9683 | 13156 | |
| 1640 | | | | | KPP03704.1 | 6078 | 12649 | 367 | 114577 | 118050 | |
| 1641 | | | | | KPP08803.1 | 6081 | 12652 | 367 | 115506 | 118979 | |
| 1642 | | | | | KPP18127.1 | 6084 | 12655 | 367 | 363358 | 366831 | |
| 1643 | | | | | KPP23764.1 | 6087 | 12658 | 367 | 9682 | 13155 | |
| 1644 | | | | | KPP34278.1 | 6090 | 12661 | 367 | 9682 | 13155 | |
| 1645 | | | | | KPP38848.1 | 6093 | 12664 | 367 | 9682 | 13155 | |
| 1646 | | | | | KPP49036.1 | 6096 | 12667 | 367 | 9683 | 13156 | |
| 1647 | | | | | KPP10064.1 | 6099 | 12670 | 367 | 9683 | 13156 | |
| 1648 | | | | | KPP41786.1 | 6102 | 12673 | 367 | 9681 | 13154 | |
| 1649 | | | | | KPP53633.1 | 6105 | 12676 | 367 | 363428 | 366901 | |
| 1650 | | | | | KRQ04752.1 | 6108 | 12679 | 367 | 280602 | 284075 | |
| 1651 | | | | | KUG91602.1 | 6111 | 12682 | 367 | 545 | 4018 | |
| 1652 | | | | | KUH02055.1 | 6114 | 12685 | 367 | 27065 | 30538 | |
| 1653 | | | | | KUS40189.1 | 6117 | 12688 | 367 | 8291 | 11764 | |
| 1654 | | | | | KUT25844.1 | 6120 | 12691 | 367 | 14510 | 17983 | |
| 1655 | | | | | KUT16912.1 | 6123 | 12694 | 367 | 2806 | 6279 | |
| 1656 | | | | | KUT50284.1 | 6126 | 12697 | 367 | 17959 | 21432 | |
| 1657 | | | | | KUU89823.1 | 6129 | 12700 | 367 | 29590 | 33063 | |
| 1658 | | | | | KUT63219.1 | 6132 | 12703 | 367 | 9401 | 12874 | |
| 1659 | | | | | KUU32868.1 | 6135 | 12706 | 367 | 28187 | 31660 | |
| 1660 | | | | | KUU24008.1 | 6138 | 12709 | 367 | 8294 | 11767 | |
| 1661 | | | | | KUV87989.1 | 6141 | 12712 | 367 | 8488 | 11961 | |
| 1662 | | | | | KUW19157.1 | 6144 | 12715 | 367 | 103408 | 106881 | |
| 1663 | | | | | KUW69108.1 | 6147 | 12718 | 367 | 60641 | 64114 | |
| 1664 | | | | | KUW47133.1 | 6150 | 12721 | 367 | 28184 | 31657 | |
| 1665 | | | | | AMG80378.1 | 6153 | 12724 | 367 | 4055455 | 4058928 | |
| 1666 | | | | | KXK90274.1 | 6156 | 12727 | 367 | 382661 | 386134 | |
| 1667 | | | | | KXP75342.1 | 6159 | 12730 | 367 | 60834 | 64307 | |
| 1668 | | | | | KXR06381.1 | 6162 | 12733 | 365 | 17180 | 20647 | |
| 1669 | | | | | KXR86338.1 | 6165 | 12736 | 365 | 17181 | 20648 | |
| 1670 | | | | | EYT05879.1 | 6168 | 12739 | 363 | 315579 | 319040 | |
| 1671 | | | | | KMV95470.1 | 6171 | 12742 | 363 | 1142870 | 1146331 | |
| 1672 | | | | | ESN46749.1 | 6174 | 12745 | 363 | 356404 | 359865 | |
| 1673 | | | | | KDL89508.1 | 6177 | 12748 | 363 | 320792 | 324253 | |
| 1674 | | | | | KMG65745.1 | 6180 | 12751 | 363 | 5112636 | 5116097 | |
| 1675 | | | | | KRR31032.1 | 6183 | 12754 | 363 | 59524 | 62985 | |
| 1676 | | | | | CVD68076.1 | 6186 | 12757 | 363 | 361657 | 365120 | |
| 1677 | | | | | CVD62391.1 | 6189 | 12760 | 363 | 246007 | 249470 | |
| 1678 | | | | | CVA98365.1 | 6192 | 12763 | 363 | 246132 | 249595 | |
| 1679 | | | | | CVB66940.1 | 6195 | 12766 | 363 | 245939 | 249402 | |
| 1680 | | | | | CVE07646.1 | 6198 | 12769 | 363 | 211862 | 215325 | |
| 1681 | | | | | CUZ34247.1 | 6201 | 12772 | 363 | 363056 | 366519 | |
| 1682 | | | | | CVH03739.1 | 6204 | 12775 | 363 | 215808 | 219271 | |
| 1683 | | | | | CUZ32693.1 | 6207 | 12778 | 363 | 361916 | 365379 | |
| 1684 | | | | | CVC01550.1 | 6210 | 12781 | 363 | 249441 | 252904 | |
| 1685 | | | | | CVC86891.1 | 6213 | 12784 | 363 | 249294 | 252757 | |
| 1686 | | | | | CVC74523.1 | 6216 | 12787 | 363 | 212906 | 216369 | |
| 1687 | | | | | CVC09195.1 | 6219 | 12790 | 363 | 362064 | 365527 | |
| 1688 | | | | | CVG04243.1 | 6222 | 12793 | 363 | 361779 | 365242 | |
| 1689 | | | | | CVB23944.1 | 6225 | 12796 | 363 | 362214 | 365677 | |
| 1690 | | | | | CVE95538.1 | 6228 | 12799 | 363 | 361839 | 365302 | |
| 1691 | | | | | CVG92146.1 | 6231 | 12802 | 363 | 214249 | 217712 | |
| 1692 | | | | | CVA05266.1 | 6234 | 12805 | 363 | 364092 | 367555 | |
| 1693 | | | | | COJ21090.1 | 6237 | 12808 | 363 | 32605 | 36068 | |
| 1694 | | | | | CNK85655.1 | 6240 | 12811 | 363 | 185197 | 188660 | |
| 1695 | | | | | KLE23457.1 | 6243 | 12814 | 361 | 207605 | 211169 | |
| 1696 | | | | | KNY42367.1 | 6246 | 12817 | 371 | 142231 | 145804 | |
| 1697 | | | | | KTL44733.1 | 6249 | 12820 | 378 | 1320810 | 1324404 | |
| 1698 | | | | | KED68171.1 | 6252 | 12823 | 378 | 48394 | 51988 | |
| 1699 | | | | | KHF16014.1 | 6255 | 12826 | 361 | 33571 | 37114 | |
| 1700 | | | | | KIT58954.1 | 6258 | 12829 | 378 | 63893 | 67196 | |
| 1701 | | | | | KIT38900.1 | 6261 | 12832 | 378 | 8473 | 11776 | |
| 1702 | | | | | KJR33560.1 | 6264 | 12835 | 378 | 440782 | 444376 | |
| 1703 | | | | | KKX83989.1 | 6267 | 12838 | 378 | 440597 | 444191 | |
| 1704 | | | | | KOE91952.1 | 6270 | 12841 | 378 | 474874 | 478468 | |
| 1705 | | | | | KOF38059.1 | 6273 | 12844 | 361 | 9424 | 12967 | |
| 1706 | | | | | KON59156.1 | 6276 | 12847 | 279 | 561224 | 564323 | |
| 1707 | | | | | KXG05867.1 | 6279 | 12850 | 378 | 440782 | 444376 | |
| 1708 | | | | | KYJ92314.1 | 6282 | 12853 | 378 | 440782 | 444376 | |
| 1709 | | | | | EMB49754.1 | 6285 | 12856 | 372 | 6770 | 9839 | |
| 1710 | | | | | EDL69244.1 | 6288 | 12859 | 378 | 167290 | 170884 | |
| 1711 | | | | | EFM85264.1 | 6291 | 12862 | 269 | 139662 | 142821 | |
| 1712 | | | | | EFM93936.1 | 6294 | 12865 | 269 | 112997 | 116156 | |
| 1713 | | | | | EFM98323.1 | 6297 | 12868 | 269 | 139455 | 142614 | |
| 1714 | | | | | ABI24730.1 | 6300 | 12871 | 269 | 481084 | 484348 | |
| 1715 | | | | | EYA48171.1 | 6303 | 12874 | 267 | 60770 | 63872 | |
| 1716 | | | | | EYA76043.1 | 6306 | 12877 | 278 | 199288 | 202423 | |
| 1717 | | | | | EYA68805.1 | 6309 | 12880 | 278 | 1 | 3131 | |
| 1718 | | | | | EYA80657.1 | 6312 | 12883 | 278 | 47013 | 50148 | |
| 1719 | | | | | CUA18410.1 | 6315 | 12886 | 278 | 2195155 | 2198290 | |
| 1720 | | | | | KDS43043.1 | 6318 | 12889 | 306 | 21582 | 24750 | |
| 1721 | | | | | KDS37969.1 | 6321 | 12892 | 306 | 65172 | 68340 | |
| 1722 | | | | | KDS73985.1 | 6324 | 12895 | 306 | 108116 | 111284 | |
| 1723 | | | | | ALF18257.1 | 6327 | 12898 | 273 | 1819401 | 1822845 | |
| 1724 | | | | | KXA24059.1 | 6330 | 12901 | 273 | 105285 | 108729 | |
| 1725 | | | | | EFG28534.1 | 6333 | 12904 | 192 | 82671 | 86075 | |
| 1726 | | | | | ADJ62018.1 | 6336 | 12907 | 293 | 542484 | 545370 | |
| 1727 | | | | | KTC70122.1 | 6339 | 12910 | 143 | 45 | 2660 | |
| 1728 | | | | | AFT94974.1 | 6342 | 12913 | 364 | 1736318 | 1739982 | |
| 1729 | | | | | EEX38487.1 | 6345 | 12916 | 361 | 46338 | 49881 | |
| 1730 | | | | | CCN78627.1 | 6348 | 12919 | 361 | 1996439 | 2000017 | |
| 1731 | | | | | CCN34619.1 | 6351 | 12922 | 361 | 70222 | 73800 | |
| 1732 | | | | | CCN40569.1 | 6354 | 12925 | 361 | 26343 | 29921 | |
| 1733 | | | | | CCN66137.1 | 6357 | 12928 | 361 | 70235 | 73813 | |
| 1734 | | | | | KGJ97947.1 | 6360 | 12931 | 373 | 43748 | 47386 | |
| 1735 | | | | | EEP52109.1 | 6363 | 12934 | 282 | 1752487 | 1755339 | |
| 1736 | | | | | AIP08273.1 | 6366 | 12937 | 282 | 87534 | 90386 | |
| 1737 | | | | | KGC46811.1 | 6369 | 12940 | 282 | 924785 | 927637 | |
| 1738 | | | | | AIV53984.1 | 6372 | 12943 | 282 | 1079826 | 1082678 | |
| 1739 | | | | | KGU70602.1 | 6375 | 12946 | 281 | 1500031 | 1502880 | |
| 1740 | | | | | KGV18647.1 | 6378 | 12949 | 282 | 3065754 | 3068606 | |
| 1741 | | | | | KGV94868.1 | 6381 | 12952 | 282 | 2670857 | 2673709 | |
| 1742 | | | | | KGV65995.1 | 6384 | 12955 | 282 | 197475 | 200327 | |
| 1743 | | | | | KGV92499.1 | 6387 | 12958 | 282 | 459725 | 462577 | |
| 1744 | | | | | KGX53111.1 | 6390 | 12961 | 282 | 123203 | 126055 | |
| 1745 | | | | | KGX64950.1 | 6393 | 12964 | 282 | 2770 | 5622 | |
| 1746 | | | | | AJX71012.1 | 6396 | 12967 | 282 | 2464517 | 2467369 | |
| 1747 | | | | | AIA69101.1 | 6399 | 12970 | 291 | 101386 | 104378 | |
| 1748 | | | | | AIK12006.1 | 6402 | 12973 | 291 | 101407 | 104399 | |
| 1749 | | | | | KMK83436.1 | 6405 | 12976 | 291 | 83001 | 85993 | |
| 1750 | | | | | KGK11297.1 | 6408 | 12979 | 378 | 1809125 | 1812719 | |
| 1751 | | | | | EAR22190.1 | 6411 | 12982 | 368 | 1014237 | 1017892 | |
| 1752 | | | | | ERG19019.1 | 6414 | 12985 | 262 | 12777 | 15389 | |
| 1753 | | | | | ERG19055.1 | 6417 | 12988 | 382 | 10170 | 13838 | |
| 1754 | | | | | ERG37345.1 | 6420 | 12991 | 219 | 39830 | 42959 | |
| 1755 | | | | | KTD25442.1 | 6423 | 12994 | 272 | 53517 | 56564 | |
| 1756 | | | | | EER46087.1 | 6426 | 12997 | 280 | 22897 | 26188 | |
| 1757 | | | | | AKZ71840.1 | 6429 | 13000 | 363 | 570853 | 574314 | |
| 1758 | | | | | KJN72850.1 | 6432 | 13003 | 363 | 57346 | 60807 | |
| 1759 | | | | | KLQ05986.1 | 6435 | 13006 | 363 | 77216 | 80677 | |
| 1760 | | | | | KHE02047.1 | 6438 | 13009 | 286 | 126996 | 129842 | |
| 1761 | | | | | KHN64403.1 | 6441 | 13012 | 286 | 308711 | 311557 | |
| 1762 | | | | | ACS83825.1 | 6444 | 13015 | 287 | 7960 | 11128 | |
| 1763 | | | | | EAS45751.1 | 6447 | 13018 | 363 | 696153 | 699714 | |
| 1764 | | | | | ALP65205.1 | 6450 | 13021 | 282 | 1243807 | 1246659 | |
| 1765 | | | | | AIO35887.1 | 6453 | 13024 | 282 | 2947387 | 2950269 | |
| 1766 | | | | | KTC93826.1 | 6456 | 13027 | 270 | 188880 | 191921 | |
| 1767 | | | | | KVP70161.1 | 6459 | 13030 | 284 | 94244 | 97102 | |
| 1768 | | | | | KVP49170.1 | 6462 | 13033 | 284 | 184358 | 187216 | |
| 1769 | | | | | KVQ79698.1 | 6465 | 13036 | 284 | 191818 | 194676 | |
| 1770 | | | | | KVR10909.1 | 6468 | 13039 | 284 | 191827 | 194685 | |
| 1771 | | | | | KVR50013.1 | 6471 | 13042 | 284 | 96265 | 99123 | |
| 1772 | | | | | KVR54360.1 | 6474 | 13045 | 284 | 94349 | 97207 | |
| 1773 | | | | | KWB45016.1 | 6477 | 13048 | 284 | 5874 | 8732 | |
| 1774 | | | | | KWB78078.1 | 6480 | 13051 | 284 | 94316 | 97174 | |
| 1775 | | | | | KWD39081.1 | 6483 | 13054 | 284 | 184358 | 187216 | |
| 1776 | | | | | KWD33066.1 | 6486 | 13057 | 284 | 191818 | 194676 | |
| 1777 | | | | | KWD38755.1 | 6489 | 13060 | 284 | 96265 | 99123 | |
| 1778 | | | | | KWO93443.1 | 6492 | 13063 | 284 | 28626 | 31484 | |
| 1779 | | | | | KVX24374.1 | 6495 | 13066 | 284 | 110871 | 113729 | |
| 1780 | | | | | KGO04270.1 | 6498 | 13069 | 259 | 857 | 3814 | |
| 1781 | | | | | ADE14193.1 | 6501 | 13072 | 377 | 1007627 | 1011302 | |
| 1782 | | | | | ADR35023.1 | 6504 | 13075 | 172 | 2375586 | 2377970 | |
| 1783 | | | | | EPF16737.1 | 6507 | 13078 | 281 | 2485440 | 2488277 | |
| 1784 | | | | | KYL32888.1 | 6510 | 13081 | 379 | 8157 | 11763 | |
| 1785 | | | | | ALS34709.1 | 6513 | 13084 | 378 | 195869 | 199469 | |
| 1786 | | | | | KOO13435.1 | 6516 | 13087 | 371 | 119459 | 123032 | |
| 1787 | | | | | KGA93589.1 | 6519 | 13090 | 195 | 161290 | 163757 | |
| 1788 | | | | | CCK76633.1 | 6522 | 13093 | 369 | 2728464 | 2731988 | |
| 1789 | | | | | CAV18533.1 | 6525 | 13096 | 362 | 1475653 | 1479223 | |
| 1790 | | | | | CEO93971.1 | 6528 | 13099 | 261 | 126 | 3047 | |
| 1791 | | | | | CDT36087.1 | 6531 | 13102 | 383 | 162067 | 165622 | |
| 1792 | | | | | CDT66912.1 | 6534 | 13105 | 383 | 1519 | 5074 | |
| 1793 | | | | | EIY57318.1 | 6537 | 13108 | 296 | 805872 | 809052 | |
| 1794 | | | | | EPR32277.1 | 6540 | 13111 | 293 | 3145 | 6033 | |
| 1795 | | | | | ENO91779.1 | 6543 | 13114 | 362 | 17111 | 20509 | |
| 1796 | | | | | EAQ53527.1 | 6546 | 13117 | 282 | 673628 | 676748 | |
| 1797 | | | | | GAA81803.1 | 6549 | 13120 | 378 | 17567 | 21167 | |
| 1798 | | | | | EEF80509.1 | 6552 | 13123 | 137 | 187507 | 190453 | |
| 1799 | | | | | KVA10029.1 | 6555 | 13126 | 281 | 247235 | 250084 | |
| 1800 | | | | | GAD76291.1 | 6558 | 13129 | 378 | 52164 | 55758 | |
| 1801 | | | | | EFM21228.1 | 6561 | 13132 | 363 | 211867 | 215330 | |
| 1802 | | | | | KJF90750.1 | 6564 | 13135 | 361 | 53142 | 56685 | |
| 1803 | | | | | KKB47517.1 | 6567 | 13138 | 262 | 187119 | 190140 | |
| 1804 | | | | | EIM72583.1 | 6570 | 13141 | 262 | 115 | 2515 | |
| 1805 | | | | | EEX64651.1 | 6573 | 13144 | 372 | 390460 | 393529 | |
| 1806 | | | | | EEY99585.1 | 6576 | 13147 | 372 | 77969 | 81548 | |
| 1807 | | | | | EGW44489.1 | 6579 | 13150 | 293 | 176059 | 178904 | |
| 1808 | | | | | KHD25431.1 | 6582 | 13153 | 378 | 154792 | 158682 | |
| 1809 | | | | | GAL03974.1 | 6585 | 13156 | 273 | 103050 | 106823 | |
| 1810 | | | | | ELA08938.1 | 6588 | 13159 | 281 | 216422 | 219641 | |
| 1811 | | | | | AEH01977.1 | 6591 | 13162 | 264 | 2395689 | 2398415 | |
| 1812 | | | | | EKM24780.1 | 6594 | 13165 | 378 | 414659 | 418285 | |
| 1813 | | | | | EOD77075.1 | 6597 | 13168 | 361 | 2838 | 6384 | |
| 1814 | | | | | KEQ18610.1 | 6600 | 13171 | 380 | 51532 | 55402 | |
| 1815 | | | | | EJF07132.1 | 6603 | 13174 | 324 | 10761 | 15775 | |
| 1816 | | | | | KWU01631.1 | 6606 | 13177 | 361 | 1104 | 4932 | |
| 1817 | | | | | KHL11031.1 | 6609 | 13180 | 282 | 2470 | 5322 | |
| 1818 | | | | | AIL32427.1 | 6612 | 13183 | 268 | 682060 | 685211 | |
| 1819 | | | | | KIM02581.1 | 6615 | 13186 | 218 | 37 | 2584 | |
| 1820 | | | | | KIM12266.1 | 6618 | 13189 | 132 | 265881 | 268795 | |
| 1821 | | | | | EJU08779.1 | 6621 | 13192 | 273 | 341208 | 344700 | |
| 1822 | | | | | AJA69894.1 | 6624 | 13195 | 265 | 3038320 | 3041220 | |
| 1823 | | | | | KRG22678.1 | 6627 | 13198 | 288 | 262834 | 265878 | |
| 1824 | | | | | KLN64109.1 | 6630 | 13201 | 376 | 105944 | 109441 | |
| 1825 | | | | | KDM93484.1 | 6633 | 13204 | 375 | 115926 | 119449 | |
| 1826 | | | | | KLE39936.1 | 6636 | 13207 | 363 | 242443 | 245906 | |
| 1827 | | | | | KPZ54444.1 | 6639 | 13210 | 378 | 57960 | 61560 | |
| 1828 | | | | | KQN43370.1 | 6642 | 13213 | 361 | 53441 | 56884 | |
| 1829 | | | | | CVJ63634.1 | 6645 | 13216 | 281 | 59414 | 62268 | |
| 1830 | | | | | CVE16997.1 | 6648 | 13219 | 363 | 215644 | 219107 | |

The ZORYA system was not found in archaea. The system is present mainly in Proteobacteria and is markedly under-represented in Gram positive bacteria (Firmicutes and Actinobacteria), suggesting that its functionality depends on the double membrane organization of Gram negative bacteria (Table 8).

The gene composition of the Zorya system may point to several hypotheses as to its mechanism of action: It is possible that the system has adopted the MotAB proton channel to achieve depolarization of membrane potential upon phage infection. Possibly, ZorC, ZorD and ZorE may be involved in the sensing and inactivation of phage DNA, and if phage inactivation fails, the ZorAB proton channel opens up, leading to membrane depolarization and cell death. Under this hypothesis Zorya may be a conditional abortive infection system.

Interestingly, although Zorya-containing cells that were infected by phage T7 did not yield phage progeny in greater than 80% of infection events, infection of Zorya-containing cells in liquid cultures led to an eventual culture collapse, suggesting that Zorya-mediated defense involves death or metabolic arrest of the infected cell (FIGS. 4G-4I).

A second, more far-fetched hypothesis as to the function of Zorya may be that ZorAB localizes to the flagellar motor and alters the activity of the flagella in response to phage infection, presumably to evade infection or to avoid infection spreading.

### Example 4

### Analysis of the Type I ZORYA System Components

Objective:

To analyze the functionality of the Type I ZORYA system.

Methods:

In order to study the four gene components of the ZORYA type I system, functionality was analyzed with mutated forms of each of the genes of the Type I ZORYA. Deletion mutants were made and the cloned vectors comprising specific gene deletions were transformed into E. coli MG1655 cells. Similarly, point mutations were generated and vectors comprising the point mutation constructs were transformed into E. coli MG1655 cells.

Results:

All four genes in the system appear to be essential for its functionality, as deletion of each of the genes resulted in loss of protection from phage infection (FIG. 4J). Moreover, the activity of the ZorAB proton channel appears to be necessary for the system's functionality, as point mutations in residues predicted to be critical for proton translocation through the channel (either ZorA: T147A/S184A or ZorB:D26N) yielded a non-functional system (FIG. 4J). Similarly, point mutations inactivating the Walker B motif of the helicase domain of ZorD, predicted to prevent ATP hydrolysis, resulted in loss of protection from phage infection.

### Example 5

### Genomic Identification and Analysis of the Thoeris System

Objective:

Identify the candidate defense system based on the enrichment of the anchor pfam08937 (TIR domain).

Methods:

The Thoeris system was one of the systems identified in the first round of prediction. The Thoeris (Egyptian protective deity of childbirth and fertility) defense system is a two-gene system that was detected based on the enrichment of pfam08937 (TIR domain) next to defense genes (FIG. 3B and FIG. 5A). The Thoeris system in some embodiments, is termed the SIR2-TIR system. The TIR domain was previously reported as associated with prokaryotic argonaute genes.

Genomic Identification and Analysis of Thoeris (SIR2-TIR) Systems—

The anchor genes family for SIR2-TIR was pfam08937, a TIR-like domain (also annotated as DUF1863). It had a defense score of 0.68 and a diversity score of 0.33. It produced several subsystems, one of which was the SIR2-TIR subsystem with a defense score of 0.58 and diversity score of 0.91. It is composed of a SIR2-domain gene, with one or more TIR-domain genes in the immediate vicinity of +/−5 genes from the SIR2 gene. Initially 78 appearances were found in the dataset that was used in the automatic prediction. Later, by manual expansion using the most up-to-date IMG dataset, and via homologs of the SIR2 gene, additional 1162 appearances were found, totaling in 1240 appearances in microbial genomes. The genes domains, properties and environment were explored using known bioinformatics tools.

Cloning of SIR2-TIR into B. subtilis BES7003—

A cloning vector for large fragments was constructed by assembling the p15a origin of replication (ori) from pACYCDuet-1 and the amyE integration cassette from plasmid pDR110 (see https://www(dot)ncbi(dot)nlm(dot)nih(dot)gov/pmc/articles/PMC3814332/#pgen.1003892.s005). The p15a ori was amplified using primers OGO174+OGO175 (a list of all primers is provided in Table 5). The amyE integration cassette was amplified using primers OGO176+OGO185. The two fragments were assembled and transformed into E. coli cells using Gibson assembly cloning kit (NEB E5510S), and assembled plasmids were verified by restriction pattern and full sequencing.

The backbone of the above described vector was amplified using primers OGO309+OGO310, adding to it a BamHI restriction site and a terminator site. The multiple cloning site of plasmid pBS1C (received from the BGSC) was amplified using primers OGO311+OGO312. Both fragments were digested using AscI and BamHI, ligated using T4 ligase and transformed into E. coli, resulting in plasmid pSG1-rfp. The plasmid was digestion verified and fully sequenced.

The SIR2-TIR locus of Bacillus cereus MSX-D12 (RefSeq accession AHEQ01000050, coordinates 16453-19685 of the forward strand, SEQ ID NO: XXXXX) was commercially synthesized (GenScript) directly into pSG1-rfp between the AscI and NotI sites of the multiple cloning site. The cloned vector was transformed into B. subtilis BEST7003 [SD1] cells, and the resulting transformants were verified by PCR and whole genome sequencing.

Phage Cultivation and Plaque Assay—

In the initial studies, phi3T, SPO1, phi29 and phi105 phages were obtained from the Bacillus Genetic Stock Center (BGSC). Phages were propagated on B. subtilis BEST7003 using the plate lysate method as described by Fortier & Moineau (Fortier and Moineau (2009) Phage Production and Maintenance of Stocks, Including Expected Stock Lifetimes. In: Clokie M. R., Kropinski A. M. (eds) Bacteriophages. Methods in Molecular Biology™, vol 501. Humana Press)

Lysate titer was determined using the small drop plaque assay method as described by Mazzocco et al. (Mazzocco et al., 2009 ibid). Efficiency of plating (EOP) was measured by performing small drop plaque assay with the same phage lysate concentrations on control bacteria and bacteria containing the SIR2-TIR system and comparing the ratio of plaque formation. Follow-up experiments included plaque assays testing the EOP against JER, Chloro, SpBeta, SPR, phi105, rho14, and SPP1.

Results:

Thoeris (Egyptian protective deity of childbirth and fertility) is a system that was detected based on the enrichment of pfam08937 (TIR domain) next to known defense genes (FIG. 5A). This domain was previously reported as associated with prokaryotic argonaute genes. The pfam08937 (DUF1863) domain was found to be significantly associated with known defense systems (defense score of 0.68). pfam08937 is a protein domain of unknown function, which was found to have structural homology with the TIR-domain (TIR=Toll-Interleukin receptor). TIR domains are known to be involved in bacterial immune system evasion in animals and plants.

As defense systems are frequently encoded by multiple genes working in concert that are co-localized in the genome (i.e. defense islands), the genetic context of the pfam08937-containing genes was analyzed. It was found that there is a subsystem in which upstream of the genes with a pfam08937 domain there is a gene with a SIR2 domain (pfam13289). Also, in many cases there was more than one TIR-domain gene in proximity (FIG. 5A). The defense score of this subsystem was 0.58, raising the hypothesis that these two (or more) genes together form an anti-phage defense system, denoted herein as SIR2-TIR. The SIR2 gene (pfam13289) encodes a protein with a median size of 483 amino acids. The TIR genes can have one of two pfams, pfam08937 or pfam13676 or pfam 08357. They encode proteins with a median size of 202 amino acids.

The first gene in the Thoeris system, denoted thsA, has NAD-binding domain that is sometimes annotated as a sirtuin (SIR2)-like domain or Macro domain. The second gene was denoted thsB, and contains the TIR domain, and can appear in one or more copies (FIG. 5A). In some versions of the Thoeris system, ThsA polypeptide has a multi-transmembrane N-terminal domain.

Two instances of this system, one from Bacillus cereus MSX-D12 (SEQ ID NO: 10; where ThsA is predicted to be cytoplasmic) and the other from Bacillus amyloliquefaciens Y2 (SEQ ID NO: 11; where ThsA is predicted to be membrane-associated) (FIG. 5B), were engineered into B. subtilis BEST7003, which does not contain an endogenous Thoeris defense system. The correct insertion of the system into the B. subtilis BEST7003 genome was verified by whole genome sequencing. The Thoeris system containing B. subtilis BEST7003 strains were challenged with multiple phages spanning the three families of the dsDNA tailed phages Caudavirales, as detailed above in the methods. The mycophages and podophages are obligatory lytic phages, while the siphophages are temperate.

The cloned systems were found to confer defense against myophages (FIGS. 3B and 3E, and FIGS. 5C and 5D). As the 3 tested myophages are very different from each other and share little gene homologies, it is possible that the Thoeris system is sensing or targeting a general feature in the biology of myophages rather than a specific sequence or genome modification. Both Thoeris genes, thsA and thsB, are essential in the system as deletion of either of them rendered the system inactive (FIG. 5C).

Interestingly, the TIR domain (acronym for Toll-Interleukin Receptor) is an important component of the innate immune systems of mammals, plants and invertebrates, where it mainly serves as a connector domain that transfers the immune signal once a molecular pattern of an offensive pathogen is sensed. In animals this domain frequently forms the intracellular portion of membrane-bound Toll-like receptors, whereas in plants it is often connected to intracellular R genes and can also be involved in direct recognition of pathogens. The findings presented here mark a common role for TIR domains in innate immunity across the three domains of life, and implies that the ancestry of this important component of eukaryotic innate immune systems may have stemmed from prokaryotic defense against phages.

The Thoeris system is broadly distributed in bacteria and archaea, and can be detected in at least 4% of the sequenced genomes we analyzed (2070 genomes; Table 9; FIG. 3H).

TABLE 9
The Thoeris Defense System and Components Thereof
This table is presented in landscape orientation. Due to the number of columns, the table
has been split into parts. Part A includes the first set of columns and therefore should
be place to the left of subsequent parts (Part B and where relevant Part C). To avoid any
doubt, the rows of the table in each part line up once the tables are placed together.
(Part A)

| | | | | | | | | | I | |
|---|---|---|---|---|---|---|---|---|---|---|
| | | | | | E | | G | H | ThsA | J |
| | A | B | C | D | infra- | F | genomic_acces- | #genes | protein_acces- | PRT SEQ |
| 1 | assembly_accession | taxid | species_taxid | organism_name | specific_name | phylum | sion | identified | sion | ID NO: |
| 2 | GCA_000166815.1 | 333849 | 1352 | Enterococcus faecium DO | DO | Firmicutes | AAAK03000018.1 | 3 | EAN09999.1 | 13220 |
| 3 | GCA_000169315.1 | 388400 | 388400 | Bacillus sp. B14905 | B14905 | Firmicutes | AAXV01000012.1 | 2 | EAZ85483.1 | 13223 |
| 4 | GCA_000172675.1 | 535206 | 1352 | Enterococcus faecium E1162 | E1162 | Firmicutes | ABQJ01000097.1 | 3 | EFF33950.1 | 13225 |
| 5 | GCA_000172915.1 | 547468 | 1352 | Enterococcus faecium U0317 | U0317 | Firmicutes | ABSW01000016.1 | 3 | EFF30624.1 | 13228 |
| 6 | GCA_000176955.1 | 596330 | 33031 | Peptoniphilus lacrimalis 315-B | 315-B | Firmicutes | ADDO01000017.1 | 2 | EFA90661.1 | 13231 |
| 7 | GCA_000147675.2 | 596315 | 341694 | Peptostreptococcus stomatis DSM 17678 | DSM 17678 | Firmicutes | ADGQ01000022.1 | 2 | EFM65150.1 | 13233 |
| 8 | GCA_000178275.1 | 702443 | 28116 | Bacteroides ovatus SD CMC 3f | SD CMC 3f | Bacteroidetes | ADMO01000088.1 | 3 | EFF51768.1 | 13235 |
| 9 | GCA_000007825.1 | 226900 | 1396 | Bacillus cereus ATCC 14579 | ATCC 14579 | Firmicutes | AE016877.1 | 2 | AAP11368.1 | 13238 |
| 10 | GCA_000179615.1 | 717606 | 59840 | Paenibacillus curdlanolyticus YK9 | YK9 | Firmicutes | AEDD01000013.1 | 3 | EFM08817.1 | 13240 |
| 11 | GCA_000226815.3 | 907491 | 714 | Aggregatibacter actinomycetemcomitans | SCC1398 | Proteobacteria | AEJP02000033.1 | 3 | KND82314.1 | 13243 |
| | | | | serotype b str. SCC1398 | | | | | | |
| 12 | GCA_000226835.3 | 907492 | 714 | Aggregatibacter actinomycetemcomitans | I23C | Proteobacteria | AEJQ02000012.1 | 3 | KOE55210.1 | 13246 |
| | | | | serotype b str. I23C | | | | | | |
| 13 | GCA_000239115.2 | 933356 | 1352 | Enterococcus faecium E4452 | E4452 | Firmicutes | AEOU01000033.1 | 3 | EHM35989.1 | 13249 |
| 14 | GCA_000298315.2 | 990285 | 1120045 | Rhizobium grahamii CCGE 502 | CCGE 502 | Proteobacteria | AEYE02000011.1 | 2 | EPE98431.1 | 13252 |
| 15 | GCA_000223275.2 | 1035188 | 249188 | Haemophilus pittmaniae HK 85 | HK 85 | Proteobacteria | AFUV01000004.1 | 3 | EGV07449.1 | 13254 |
| 16 | GCA_000231965.2 | 760839 | 1313 | Streptococcus pneumoniae GA47502 | GA47502 | Firmicutes | AGNV01000008.1 | 3 | EHD29827.1 | 13257 |
| 17 | GCA_000239555.2 | 904766 | 1280 | Staphylococcus aureus subsp. | 21331 | Firmicutes | AGTV01000033.1 | 3 | EHM75393.1 | 13260 |
| | | | | aureus 21331 | | | | | | |
| 18 | GCA_000259915.1 | 1089447 | 714 | Aggregatibacter actinomycetemcomitans | RhAA1 | Proteobacteria | AHGR01000001.1 | 3 | EHK91559.1 | 13263 |
| | | | | RhAA1 | | | | | | |
| 19 | GCA_000401595.1 | 1137132 | 1280 | Staphylococcus aureus subsp. | 112808A | Firmicutes | AHZK01000035.1 | 3 | EOR48149.1 | 13266 |
| | | | | aureus 112808A | | | | | | |
| 20 | GCA_000257805.1 | 1095735 | 28037 | Streptococcus mitis SK616 | SK616 | Firmicutes | AICR01000116.1 | 3 | EID19247.1 | 13269 |
| 21 | GCA_000252405.2 | 1155079 | 1280 | Staphylococcus aureus subsp. | DR10 | Firmicutes | AIDT01000006.1 | 3 | EIA14317.1 | 13272 |
| | | | | aureus DR10 | | | | | | |
| 22 | GCA_000257705.1 | 1095750 | 467210 | Lachnoanaerobaculum saburreum F0468 | F0468 | Firmicutes | AJGH01000082.1 | 2 | EIC95439.1 | 13275 |
| 23 | GCA_000256585.3 | 406819 | 406819 | Burkholderia sp. SJ98 | SJ98 | Proteobacteria | AJHK02000012.1 | 2 | EKS70339.1 | 13277 |
| 24 | GCA_000332935.2 | 1167009 | 714 | Aggregatibacter actinomycetemcomitans | SCC4092 | Proteobacteria | AJMF02000026.1 | 3 | KOE52661.1 | 13279 |
| | | | | serotype b str. SCC4092 | | | | | | |
| 25 | GCA_000332955.2 | 1167008 | 714 | Aggregatibacter actinomycetemcomitans | S23A | Proteobacteria | AJMH02000015.1 | 3 | KOE56127.1 | 13282 |
| | | | | serotype b str. S23A | | | | | | |
| 26 | GCA_000262265.1 | 1095744 | 735 | Haemophilus parahaemolyticus HK385 | HK385 | Proteobacteria | AJSW01000041.1 | 3 | EIJ69644.1 | 13285 |
| 27 | GCA_000264155.1 | 752788 | 562 | Escherichia coli 75 | 75 | Proteobacteria | AJWT01000218.1 | 2 | EIL59628.1 | 13288 |
| 28 | GCA_000279285.1 | 991946 | 666 | Vibrio cholerae HE-45 | HE-45 | Proteobacteria | ALED01000025.1 | 2 | EJH65283.1 | 13290 |
| 29 | GCA_000290475.1 | 1154930 | 1311 | Streptococcus agalactiae GB00679 | GB00679 | Firmicutes | ALTY01000018.1 | 2 | EPV35489.1 | 13292 |
| 30 | GCA_000067165.1 | 448385 | 56 | Sorangium cellulosum So ce56 | So ce 56 | Proteobacteria | AM746676.1 | 2 | CAN93966.1 | 13294 |
| 31 | GCA_000009585.1 | 523796 | 1280 | Staphylococcus aureus subsp. | type strain: | Firmicutes | AM990992.1 | 3 | CAQ50582.1 | 13296 |
| | | | | aureus ST398 | ST398 | | | | | |
| 32 | GCA_000300515.1 | 1231190 | 721133 | Nitratireductor indicus C115 | C115 | Proteobacteria | AMSI01000038.1 | 3 | EKF39860.1 | 13299 |
| 33 | GCA_000468975.2 | 1232203 | 1428 | Bacillus thuringiensis serovar | Leapi01 | Firmicutes | AMXS02000100.1 | 2 | ETE93554.1 | 13302 |
| | | | | aizawai str. Leapi01 | | | | | | |
| 34 | GCA_000468995.3 | 1238885 | 1428 | Bacillus thuringiensis serovar | Hu4-2 | Firmicutes | AMXT02000117.1 | 2 | ETE97368.1 | 13304 |
| | | | | aizawai str. Hu4-2 | | | | | | |
| 35 | GCA_000410435.1 | 1256211 | 1597 | Lactobacillus paracasei subsp. | CNCM | Firmicutes | ANKH01000630.1 | 2 | EPC81228.1 | 13306 |
| | | | | paracasei CNCM I-4649 | I-4649 | | | | | |
| 36 | GCA_000326585.1 | 1169377 | 562 | Escherichia coli KTE113 | KTE113 | Proteobacteria | ANXQ01000026.1 | 2 | ELI26319.1 | 13308 |
| 37 | GCA_000326445.1 | 1169399 | 562 | Escherichia coli KTE168 | KTE168 | Proteobacteria | ANYM01000035.1 | 2 | ELJ29820.1 | 13310 |
| 38 | GCA_000341815.1 | 1278077 | 334542 | Rhodococcus qingshengii BKS 20-40 | BKS 20-40 | Actinobacteria | AODN01000101.1 | 3 | EME14866.1 | 13312 |
| 39 | GCA_000342205.1 | 1263833 | 615 | Serratia marcescens VGH107 | VGH107 | Proteobacteria | AORJ01000083.1 | 2 | EMF02811.1 | 13315 |
| 40 | GCA_000828775.1 | 1334564 | 615 | Serratia marcescens SM39 | SM39 | Proteobacteria | AP013063.1 | 2 | BAO35114.1 | 13317 |
| 41 | GCA_000828875.1 | 758796 | 758796 | Burkholderia sp. RPE67 | RPE67 | Proteobacteria | AP014579.1 | 2 | BAO91362.1 | 13319 |
| 42 | GCA_001547895.1 | 1500686 | 1500686 | Pseudomonas sp. Os17 | Os17 | Proteobacteria | AP014627.1 | 5 | BAQ74702.1 | 13321 |
| 43 | GCA_000372645.1 | 1292034 | 155892 | Caulobacter crescentus OR37 | OR37 | Proteobacteria | APMP01000003.1 | 3 | ENZ83250.1 | 13326 |
| 44 | GCA_000445085.1 | 428994 | 428994 | Sphingobium sp. HDIP04 | HDIP04 | Proteobacteria | ATDO01000049.1 | 2 | EQB07550.1 | 13329 |
| 45 | GCA_000416235.1 | 911242 | 911242 | Pseudomonas sp. CFII64 | CFII64 | Proteobacteria | ATLO01000035.1 | 2 | EPJ82189.1 | 13331 |
| 46 | GCA_000421925.1 | 1346790 | 420233 | Sphingobium chinhatense IP26 | IP26 | Proteobacteria | AUDA01000159.1 | 2 | EPR14527.1 | 13333 |
| 47 | GCA_000455725.2 | 1344577 | 1280 | Staphylococcus aureus S1 | S1 | Firmicutes | AUPS01000015.1 | 3 | EQM91604.1 | 13335 |
| 48 | GCA_000443245.1 | 1344578 | 1280 | Staphylococcus aureus S130 | S130 | Firmicutes | AUPT01000016.1 | 3 | EPZ04960.1 | 13338 |
| 49 | GCA_000443265.1 | 1344579 | 1280 | Staphylococcus aureus S123 | S123 | Firmicutes | AUPU01000015.1 | 3 | EPZ06671.1 | 13341 |
| 50 | GCA_000443125.1 | 1344580 | 1280 | Staphylococcus aureus S100 | S100 | Firmicutes | AUPV01000016.1 | 3 | EPZ03686.1 | 13344 |
| 51 | GCA_000443285.1 | 1344581 | 1280 | Staphylococcus aureus S94 | S94 | Firmicutes | AUPW01000016.1 | 3 | EPZ11495.1 | 13347 |
| 52 | GCA_000477515.1 | 1366052 | 1613 | Lactobacillus fermentum MTCC 8711 | MTCC 8711 | Firmicutes | AVAB01000001.1 | 2 | EQC60101.1 | 13350 |
| 53 | GCA_000449945.2 | 1151319 | 1496 | Peptoclostridium difficile DA00065 | DA00065 | Firmicutes | AVIU01000072.1 | 2 | EQG20075.1 | 13352 |
| 54 | GCA_000452125.2 | 1151436 | 1496 | Peptoclostridium difficile P71 | P71 | Firmicutes | AVMW01000033.1 | 2 | EQK22995.1 | 13354 |
| 55 | GCA_000516615.2 | 1343073 | 470 | Acinetobacter baumannii CI78 | CI78 | Proteobacteria | AVOE01000023.1 | 6 | ETR94925.1 | 13356 |
| 56 | GCA_000454165.1 | 1238193 | 670 | Vibrio parahaemolyticus VP2007-095 | VP2007-095 | Proteobacteria | AVOI01000015.1 | 3 | EQL89245.1 | 13362 |
| 57 | GCA_000465615.2 | 1357297 | 615 | Serratia marcescens EGD-HP20 | EGD-HP20 | Proteobacteria | AVSR01000008.1 | 2 | ERH73216.1 | 13365 |
| 58 | GCA_000478965.1 | 1111454 | 1111454 | Megasphaera sp. BV3C16-1 | BV3C16-1 | Firmicutes | AWXA01000003.1 | 2 | ERT62562.1 | 13367 |
| 59 | GCA_000478985.1 | 1111134 | 1111134 | Peptoniphilus sp. BV3C26 | BV3C26 | Firmicutes | AWXB01000008.1 | 3 | ERT61554.1 | 13369 |
| 60 | GCA_000478985.1 | 1111134 | 1111134 | Peptoniphilus sp. BV3C26 | BV3C26 | Firmicutes | AWXB01000046.1 | 2 | ERT58436.1 | 13372 |
| 61 | GCA_000478865.2 | 1388760 | 303 | Pseudomonas putida SJ3 | SJ3 | Proteobacteria | AXDX02000039.1 | 5 | ERT19168.2 | 13374 |
| 62 | GCA_000496325.1 | 1408193 | 287 | Pseudomonas aeruginosa VRFPA05 | VRFPA05 | Proteobacteria | AXZJ01000100.1 | 3 | ESR69982.1 | 13379 |
| 63 | GCA_000648195.1 | 1400808 | 1351 | Enterococcus faecalis NY9 | NY9 | Firmicutes | AYOL01000045.1 | 3 | KAJ84751.1 | 13382 |
| 64 | GCA_000648195.1 | 1400808 | 1351 | Enterococcus faecalis NY9 | NY9 | Firmicutes | AYOL01000110.l | 2 | KAJ82225.1 | 13385 |
| 65 | GCA_000506425.2 | 1415162 | 1061 | Rhodobacter capsulatus B6 | B6 | Proteobacteria | AYQA01000057.1 | 2 | ETD82032.1 | 13387 |
| 66 | GCA_000503195.1 | 1419876 | 1419876 | Blastomonas sp. CACIA14H2 | | Proteobacteria | AYSC01000003.1 | 3 | ESZ88708.1 | 13389 |
| 67 | GCA_000510425.1 | 1161902 | 35518 | Eubacterium nodatum ATCC 33099 | ATCC 33099 | Firmicutes | AZKM01000022.1 | 2 | ETJ99320.1 | 13392 |
| 68 | GCA_001596225.1 | 1434263 | 714 | Aggregatibacter actinomycetemcomitans | SA2149 | Proteobacteria | AZTT01000065.1 | 3 | KYK73940.1 | 13394 |
| | | | | serotype e str. SA2149 | | | | | | |
| 69 | GCA_000512995.1 | 936596 | 936596 | Lachnoanaerobaculum sp. MSX33 | MSX33 | Firmicutes | AZXX01000025.1 | 2 | ETO98932.1 | 13397 |
| 70 | GCA_000513095.1 | 1236970 | 127891 | Bacillus wakoensis JCM 9140 | JCM 9140 | Firmicutes | BAUT01000052.1 | 2 | GAE27470.1 | 13399 |
| 71 | GCA_000787715.1 | 1219049 | 28213 | Sphingomonas parapaucimobilis NBRC 15100 | NBRC 15100 | Proteobacteria | BBPI01000099.1 | 3 | GAM02795.1 | 13401 |
| 72 | GCA_000195675.1 | 257310 | 518 | Bordetella bronchiseptica RB50 | RB50 | Proteobacteria | BX470250.1 | 3 | CAE30982.1 | 13404 |
| 73 | GCA_000355725.1 | 1118157 | 40324 | Stenotrophomonas maltophilia RA8 | RA8 | Proteobacteria | CALM01001187.1 | 3 | CCP18523.1 | 13407 |
| 74 | GCA_000985975.1 | 323656 | 399 | Neorhizobium galegae bv. officinalis | | Proteobacteria | CCRK01000014.1 | 2 | CDZ53063.1 | 13410 |
| 75 | GCA_000820425.1 | 1363 | 1363 | Lactococcus garvieae | M14 | Firmicutes | CCXC01000004.1 | 2 | CEF52297.1 | 13412 |
| 76 | GCA_001113365.1 | 1313 | 1313 | Streptococcus pneumoniae | SMRU2014 | Firmicutes | CKYA01000015.1 | 2 | CJD59524.1 | 13414 |
| 77 | GCA_000161115.1 | 526978 | 1396 | Bacillus cereus BDRD-Cer4 | BDRD-Cer4 | Firmicutes | CM000726.1 | 2 | EEL09527.1 | 13416 |
| 78 | GCA_000161395.1 | 526993 | 1396 | Bacillus cereus AH1272 | AH1272 | Firmicutes | CM000740.1 | 3 | EEL84782.1 | 13418 |
| 79 | GCA_000003955.1 | 526994 | 1396 | Bacillus cereus AH1273 | AH1273 | Firmicutes | CM000741.1 | 3 | EEL90114.1 | 13421 |
| 80 | GCA_000159695.1 | 525282 | 1260 | Finegoldia magna ATCC 53516 | ATCC 53516 | Firmicutes | CM000955.1 | 2 | EFH92719.1 | 13424 |
| 81 | GCA_000183885.1 | 702456 | 1642 | Listeria innocua FSLS4-378 | FSLS4-378 | Firmicutes | CM001048.1 | 2 | EFR92171.1 | 13426 |
| 82 | GCA_000263675.2 | 1038924 | 294 | Pseudomonas fluorescens SS101 | SS101 | Proteobacteria | CM001513.1 | 3 | EIK62927.1 | 13428 |
| 83 | GCA_000012605.1 | 317025 | 39765 | Thiomicrospira crunogena XCL-2 | XCL-2 | Proteobacteria | CP000109.2 | 4 | ABB40969.1 | 13431 |
| 84 | GCA_000013605.1 | 338969 | 192843 | Rhodoferax ferrireducens T118 | DSM 15236 | Proteobacteria | CP000267.1 | 2 | ABD68809.1 | 13435 |
| 85 | GCA_000013625.1 | 375286 | 375286 | Janthinobacterium sp. Marseille | Marseille | Proteobacteria | CP000269.1 | 3 | ABR91460.1 | 13437 |
| 86 | GCA_000014665.1 | 60481 | 60481 | Shewanella sp. MR-7 | MR-7 | Proteobacteria | CP000444.1 | 3 | ABI42036.1 | 13440 |
| 87 | GCA_000015885.1 | 416269 | 715 | Actinobacillus pleuropneumoniae | L20 | Proteobacteria | CP000569.1 | 3 | ABN74271.1 | 13443 |
| | | | | serovar 5b str. L20 | | | | | | |
| 88 | GCA_000016125.1 | 402880 | 39152 | Methanococcus maripaludis C5 | C5 | Euryarchaeota | CP000609.1 | 2 | ABO35466.1 | 13446 |
| 89 | GCA_000016405.1 | 349102 | 1063 | Rhodobacter sphaeroides ATCC 17025 | ATCC 17025 | Proteobacteria | CP000665.1 | 3 | ABP73209.1 | 13448 |
| 90 | GCA_000016965.1 | 290402 | 1520 | Clostridium beijerinckii NCIMB8052 | NCIMB 8052 | Firmicutes | CP000721.1 | 5 | ABR34958.1 | 13451 |
| 91 | GCA_000017145.1 | 366394 | 110321 | Sinorhizobium medicae WSM419 | WSM419 | Proteobacteria | CP000741.1 | 2 | ABR65059.1 | 13456 |
| 92 | GCA_000019445.1 | 390235 | 303 | Pseudomonas putida W619 | W619 | Proteobacteria | CP000949.1 | 5 | ACA73845.1 | 13458 |
| 93 | GCA_000019925.1 | 398577 | 152480 | Burkholderia ambifaria MC40-6 | MC40-6 | Proteobacteria | CP001026.1 | 3 | ACB66894.1 | 13463 |
| 94 | GCA_000146265.2 | 668336 | 714 | Aggregatibacter actinomycetemcomitans | D11S-1 | Proteobacteria | CP001733.1 | 3 | ACX81594.1 | 13466 |
| | | | | D11S-1 | | | | | | |
| 95 | GCA_000024405.1 | 526218 | 826 | Sebaldella termitidis ATCC 33386 | ATCC 33386 | Fusobacteria | CP001740.1 | 2 | ACZ11005.1 | 13469 |
| 96 | GCA_000025345.1 | 526225 | 1861 | Geodermatophilus obscurus DSM 43160 | DSM 43160 | Actinobacteria | CP001867.1 | 3 | ADB76077.1 | 13471 |
| 97 | GCA_000192635.1 | 861208 | 861208 | Agrobacterium sp. H13-3 | H13-3 | Proteobacteria | CP002250.1 | 4 | ADY67727.1 | 13474 |
| 98 | GCA_000166135.1 | 298654 | 298654 | Frankia sp. Eul1c | Eul1c | Actinobacteria | CP002299.1 | 2 | ADP84996.1 | 13478 |
| 99 | GCA_000195575.1 | 208596 | 208596 | Carnobacterium sp. 17-4 | 17-4 | Firmicutes | CP002563.1 | 3 | AEB29797.1 | 13480 |
| 100 | GCA_000194745.1 | 999541 | 28095 | Burkholderia gladioli BSR3 | BSR3 | Proteobacteria | CP002599.1 | 6 | AEA61152.1 | 13483 |
| 101 | GCA_000258685.1 | 1155084 | 1280 | Staphylococcus aureus subsp. | 71193 | Firmicutes | CP003045.1 | 3 | AFH70387.1 | 13489 |
| | | | | aureus 71193 | | | | | | |
| 102 | GCA_000241025.2 | 754507 | 714 | Aggregatibacter actinomycetemcomitans | ANH9381 | Proteobacteria | CP003099.1 | 3 | AEW76524.1 | 13492 |
| | | | | ANH9381 | | | | | | |
| 103 | GCA_000239195.1 | 347495 | 1396 | Bacillus cereus F837/76 | F837/76 | Firmicutes | CP003187.1 | 2 | AEW57570.1 | 13495 |
| 104 | GCA_000264455.2 | 1110502 | 171437 | Tistrella mobilis KA081020-065 | KA081020-065 | Proteobacteria | CP003236.1 | 4 | AFK54067.1 | 13497 |
| 105 | GCA_000747565.1 | 488142 | 488142 | Serratia sp. SCBI | SCBI | Proteobacteria | CP003424.1 | 2 | AIM23120.1 | 13501 |
| 106 | GCA_000230695.3 | 666685 | 666685 | Rhodanobacter denitrificans | 2APBS1 | Proteobacteria | CP003470.1 | 2 | AGG89637.1 | 13503 |
| 107 | GCA_000174395.2 | 333849 | 1352 | Enterococcus faecium DO | DO | Firmicutes | CP003583.1 | 3 | AFK58216.1 | 13505 |
| 108 | GCA_000281215.1 | 1196325 | 303 | Pseudomonas putida DOT-T1E | DOT-TIE | Proteobacteria | CP003734.1 | 4 | AF049035.1 | 13508 |
| 109 | GCA_000296595.1 | 1229492 | 1280 | Staphylococcus aureus 08BA02176 | 08BA02176 | Firmicutes | CP003808.1 | 3 | AFR74120.1 | 13512 |
| 110 | GCA_000599545.1 | 543736 | 37919 | Rhodococcus opacus PD630 | PD630 | Actinobacteria | CP003949.1 | 2 | AHK33011.1 | 13515 |
| 111 | GCA_000336425.1 | 435998 | 615 | Serratia marcescens WW4 | WW4 | Proteobacteria | CP003959.1 | 2 | AGE19433.1 | 13517 |
| 112 | GCA_000257545.3 | 1147786 | 75612 | Pseudomonas mandelii JR-1 | JR-1 | Proteobacteria | CP005960.1 | 4 | AHZ68662.1 | 13519 |
| 113 | GCA_000401555.1 | 1288394 | 644 | Aeromonas hydrophila ML09-119 | ML09-119 | Proteobacteria | CP005966.1 | 2 | AGM44765.1 | 13523 |
| 114 | GCA_000338115.2 | 1284663 | 1590 | Lactobacillus plantarum ZJ316 | ZJ316 | Firmicutes | CP006247.1 | 2 | AGS26695.1 | 13525 |
| 115 | GCA_000176835.2 | 682634 | 82996 | Serratia plymuthica 4Rx13 | 4Rx13 | Proteobacteria | CP006250.1 | 2 | AGO56414.1 | 13527 |
| 116 | GCA_000418305.1 | 571800 | 571800 | Psychrobacter sp. G | G | Proteobacteria | CP006265.1 | 3 | AGP48697.1 | 13529 |
| 117 | GCA_000767745.1 | 864803 | 1520 | Clostridium beijerinckii ATCC 35702 | SA-1 | Firmicutes | CP006777.1 | 5 | AIU01497.1 | 13532 |
| 118 | GCA_000504585.1 | 1380774 | 1380774 | Pandoraea sp. RB-44 | RB-44 | Proteobacteria | CP006938.1 | 3 | AHB77236.1 | 13537 |
| 119 | GCA_000835085.1 | 1124992 | 2095 | Mycoplasma capricolum subsp. | 87001 | Tenericutes | CP006959.1 | 2 | AJK51071.1 | 13540 |
| | | | | capripneumoniae 87001 | | | | | | |
| 120 | GCA_000510285.1 | 1435044 | 76759 | Pseudomonas monteilii SB3078 | SB3078 | Proteobacteria | CP006978.1 | 3 | AHC80715.1 | 13542 |
| 121 | GCA_000510325.1 | 1435058 | 76759 | Pseudomonas monteilii SB3101 | SB3101 | Proteobacteria | CP006979.1 | 3 | AHC86147.1 | 13545 |
| 122 | GCA_000604045.1 | 272556 | 714 | Aggregatibacter actinomycetemcomitans | HK1651 | Proteobacteria | CP007502.1 | 3 | AHN71072.1 | 13548 |
| | | | | HK1651 | | | | | | |
| 123 | GCA_001191585.1 | 272556 | 714 | Aggregatibacter actinomycetemcomitans | HK1651 | Proteobacteria | CP008984.1 | 3 | AKU28345.1 | 13551 |
| | | | | HK1651 | | | | | | |
| 124 | GCA_000834215.1 | 1454377 | 29484 | Yersinia frederiksenii Y225 | Y225 | Proteobacteria | CP009364.1 | 3 | AJI87975.1 | 13554 |
| 125 | GCA_000758725.1 | 189426 | 189426 | Paenibacillus odorifer | DSM 15391 | Firmicutes | CP009428.1 | 3 | AIQ75144.1 | 13557 |
| 126 | GCA_000477035.2 | 1348585 | 76594 | Cellulophaga baltica NN016038 | NN016038 | Bacteroidetes | CP009887.1 | 2 | AIY12608.1 | 13560 |
| 127 | GCA_000834865.1 | 28152 | 28152 | Yersinia kristensenii | Y231 | Proteobacteria | CP009997.1 | 3 | AJJ37542.1 | 13562 |
| 128 | GCA_000831585.1 | 70775 | 70775 | Pseudomonas plecoglossicida | NyZ12 | Proteobacteria | CP010359.1 | 3 | AJG12527.1 | 13565 |
| 129 | GCA_000961035.1 | 35554 | 35554 | Geobacter sulfurreducens | AM-1 | Proteobacteria | CP010430.1 | 3 | AJY68818.1 | 13568 |
| 130 | GCA_000934565.1 | 294 | 294 | Pseudomonas fluorescens | PCL1751 | Proteobacteria | CP010896.1 | 2 | AJP52231.1 | 13571 |
| 131 | GCA_000934525.1 | 589873 | 589873 | Alteromonas australica | DE170 | Proteobacteria | CP010912.1 | 2 | AJP42564.1 | 13573 |
| 132 | GCA_001023535.1 | 587753 | 587753 | Pseudomonas chlororaphis | UFB2 | Proteobacteria | CP011020.1 | 3 | AKK01414.1 | 13575 |
| 133 | GCA_000973085.1 | 187101 | 187101 | Sneathia amnii | SN35 | Fusobacteria | CP011280.1 | 4 | AKC95051.1 | 13578 |
| 134 | GCA_001043175.1 | 330734 | 330734 | Marinobacter psychrophilus | 20041 | Proteobacteria | CP011494.1 | 2 | AKO52328.1 | 13582 |
| 135 | GCA_001266795.1 | 1671721 | 1671721 | Marinobacter sp. CP1 | CP1 | Proteobacteria | CP011929.1 | 2 | AKV98647.1 | 13584 |
| 136 | GCA_001294565.1 | 615 | 615 | Serratia marcescens | SmUNAM836 | Proteobacteria | CP012685.1 | 2 | ALE97361.1 | 13586 |
| 137 | GCA_001310255.1 | 1532555 | 1532555 | Brevundimonas sp. DS20 | DS20 | Proteobacteria | CP012897.1 | 4 | ALJ08795.1 | 13588 |
| 138 | GCA_001594245.1 | 714 | 714 | Aggregatibacter actinomycetemcomitans | VT1169 | Proteobacteria | CP012958.1 | 3 | AMQ92899.1 | 13592 |
| 139 | GCA_001414055.1 | 69395 | 69395 | Caulobacter henricii | CB4 | Proteobacteria | CP013002.1 | 3 | ALL13164.1 | 13595 |
| 140 | GCA_001455365.1 | 644 | 644 | Aeromonas hydrophila | JBN2301 | Proteobacteria | CP013178.1 | 3 | ALQ62810.1 | 13598 |
| 141 | GCA_001465635.1 | 1280 | 1280 | Staphylococcus aureus | RIVM1295 | Firmicutes | CP013616.1 | 3 | ALS70450.1 | 13601 |
| 142 | GCA_001465675.1 | 1280 | 1280 | Staphylococcus aureus | RIVM1607 | Firmicutes | CP013619.1 | 3 | ALS73061.1 | 13604 |
| 143 | GCA_001465755.1 | 1280 | 1280 | Staphylococcus aureus | RIVM3897 | Firmicutes | CP013621.1 | 3 | ALS86458.1 | 13607 |
| 144 | GCA_001518915.1 | 1763998 | 1763998 | Rheinheimera sp. F8 | F8 | Proteobacteria | CP013656.1 | 2 | ALZ77344.1 | 13610 |
| 145 | GCA_001518775.1 | 644 | 644 | Aeromonas hydrophila | D4 | Proteobacteria | CP013965.1 | 3 | ALZ79510.1 | 13612 |
| 146 | GCA_001518735.1 | 1352 | 1352 | Enterococcus faecium | 6E6 | Firmicutes | CP013994.1 | 3 | ALZ52747.1 | 13615 |
| 147 | GCA_001561955.1 | 991789 | 28446 | [Clostridium] propionicum DSM 1682 | X2 | Firmicutes | CP014223.1 | 4 | AMJ40926.1 | 13618 |
| 148 | GCA_001560895.1 | 28037 | 28037 | Streptococcus mitis | SVGS_061 | Firmicutes | CP014326.1 | 3 | AMH88749.1 | 13622 |
| 149 | GCA_001515585.2 | 303 | 303 | Pseudomonas putida | 1A00316 | Proteobacteria | CP014343.1 | 2 | AMK33280.1 | 13625 |
| 150 | GCA_001112925.1 | 685706 | 685706 | Yersinia nurmii | type strain: | Proteobacteria | CPYD01000022.1 | 2 | CNF27344.1 | 13627 |
| | | | | | CIP110231 | | | | | |
| 151 | GCA_001140625.1 | 633 | 633 | Yersinia pseudotuberculosis | R819 | Proteobacteria | CPZI01000008.1 | 3 | CNF62528.1 | 13629 |
| 152 | GCA_001121225.1 | 29484 | 29484 | Yersinia frederiksenii | IP25924 | Proteobacteria | CQBF01000033.1 | 3 | CNI82010.1 | 13632 |
| 153 | GCA_001114105.1 | 367190 | 367190 | Yersinia similis | Y233 | Proteobacteria | CQBK01000054.1 | 3 | CNI72044.1 | 13635 |
| 154 | GCA_001368815.1 | 1462524 | 1462524 | Paraliobacillus sp. PM-2 | PM-2 | Firmicutes | CTEI01000001.1 | 2 | CQR45868.1 | 13638 |
| 155 | GCA_001222805.1 | 630 | 630 | Yersinia enterocolitica | YE09/03 | Proteobacteria | CTKQ01000009.1 | 5 | CQJ38356.1 | 13640 |
| 156 | GCA_001499695.1 | 40324 | 40324 | Stenotrophomonas maltophilia | | Proteobacteria | CVIU01000014.1 | 4 | CRD50721.1 | 13645 |
| 157 | GCA_001406815.1 | 301302 | 301302 | Roseburia faecis | M72 | Firmicutes | CVRR01000033.1 | 2 | CRL40092.1 | 13649 |
| 158 | GCA_001177845.1 | 148942 | 1336 | Streptococcus equi subsp. equi | EQUI0003 | Firmicutes | CVWY01000044.1 | 2 | CRR05859.1 | 13651 |
| 159 | GCA_001179425.1 | 148942 | 1336 | Streptococcus equi subsp. equi | EQUI0005 | Firmicutes | CVXE01000065.1 | 2 | CRR37079.1 | 13653 |
| 160 | GCA_001179085.1 | 148942 | 1336 | Streptococcus equi subsp. equi | EQUI0004 | Firmicutes | CVXF01000062.1 | 2 | CRR43612.1 | 13655 |
| 161 | GCA_001178765.1 | 148942 | 1336 | Streptococcus equi subsp. equi | EQUI0007 | Firmicutes | CVXG01000062.1 | 2 | CRR34883.1 | 13657 |
| 162 | GCA_001178485.1 | 148942 | 1336 | Streptococcus equi subsp. equi | EQUI0006 | Firmicutes | CVXI01000065.1 | 2 | CRR50108.1 | 13659 |
| 163 | GCA_001175105.1 | 148942 | 1336 | Streptococcus equi subsp. equi | EQUI0008 | Firmicutes | CVXJ01000058.1 | 2 | CRR37365.1 | 13661 |
| 164 | GCA_001175965.1 | 148942 | 1336 | Streptococcus equi subsp. equi | EQUI0009 | Firmicutes | CVXK01000003.1 | 2 | CRR01737.1 | 13663 |
| 165 | GCA_001175845.1 | 148942 | 1336 | Streptococcus equi subsp. equi | EQUI0010 | Firmicutes | CVXM01000058.1 | 2 | CRR52905.1 | 13665 |
| 166 | GCA_001176705.1 | 148942 | 1336 | Streptococcus equi subsp. equi | EQUI0011 | Firmicutes | CVX001000058.1 | 2 | CRR41690.1 | 13667 |
| 167 | GCA_001178165.1 | 148942 | 1336 | Streptococcus equi subsp. equi | EQUI0033 | Firmicutes | CVXQ01000063.1 | 2 | CRS57164.1 | 13669 |
| 168 | GCA_001178685.1 | 148942 | 1336 | Streptococcus equi subsp. equi | EQUI0035 | Firmicutes | CVXR01000068.1 | 2 | CRS58226.1 | 13671 |
| 169 | GCA_001178905.1 | 148942 | 1336 | Streptococcus equi subsp. equi | EQUI0036 | Firmicutes | CVXS01000068.1 | 2 | CRS57780.1 | 13673 |
| 170 | GCA_001178025.1 | 148942 | 1336 | Streptococcus equi subsp. equi | EQUI0034 | Firmicutes | CVXT01000066.1 | 2 | CRS58151.1 | 13675 |
| 171 | GCA_001178585.1 | 148942 | 1336 | Streptococcus equi subsp. equi | EQUI0037 | Firmicutes | CVXU01000042.1 | 2 | CRS55109.1 | 13677 |
| 172 | GCA_001175585.1 | 148942 | 1336 | Streptococcus equi subsp. equi | EQUI0039 | Firmicutes | CVXV01000043.1 | 2 | CRS48588.1 | 13679 |
| 173 | GCA_001175365.1 | 148942 | 1336 | Streptococcus equi subsp. equi | EQUI0040 | Firmicutes | CVXW01000043.1 | 2 | CRS51337.1 | 13681 |
| 174 | GCA_001175305.1 | 148942 | 1336 | Streptococcus equi subsp. equi | EQUI0038 | Firmicutes | CVXX01000041.1 | 2 | CRS48392.1 | 13683 |
| 175 | GCA_001175645.1 | 148942 | 1336 | Streptococcus equi subsp. equi | EQUI0041 | Firmicutes | CVXY01000044.1 | 2 | CRS50262.1 | 13685 |
| 176 | GCA_001176045.1 | 148942 | 1336 | Streptococcus equi subsp. equi | EQUI0042 | Firmicutes | CVXZ01000043.1 | 2 | CRS50718.1 | 13687 |
| 177 | GCA_001176505.1 | 148942 | 1336 | Streptococcus equi subsp. equi | EQUI0043 | Firmicutes | CVYA01000044.1 | 2 | CRS54427.1 | 13689 |
| 178 | GCA_001176825.1 | 148942 | 1336 | Streptococcus equi subsp. equi | EQUI0044 | Firmicutes | CVYB01000042.1 | 2 | CRS57080.1 | 13691 |
| 179 | GCA_001176965.1 | 148942 | 1336 | Streptococcus equi subsp. equi | EQUI0045 | Firmicutes | CVYC01000044.1 | 2 | CRS58973.1 | 13693 |
| 180 | GCA_001177245.1 | 148942 | 1336 | Streptococcus equi subsp. equi | EQUI0046 | Firmicutes | CVYD01000043.1 | 2 | CRS51127.1 | 13695 |
| 181 | GCA_001177205.1 | 148942 | 1336 | Streptococcus equi subsp. equi | EQUI0047 | Firmicutes | CVYE01000045.1 | 2 | CRS51981.1 | 13697 |
| 182 | GCA_001177785.1 | 148942 | 1336 | Streptococcus equi subsp. equi | EQUI0220 | Firmicutes | CVYF01000064.1 | 2 | CRS77740.1 | 13699 |
| 183 | GCA_001179185.1 | 148942 | 1336 | Streptococcus equi subsp. equi | EQUI0222 | Firmicutes | CVYG01000060.1 | 2 | CRS83912.1 | 13701 |
| 184 | GCA_001178625.1 | 148942 | 1336 | Streptococcus equi subsp. equi | EQUI0048 | Firmicutes | CVYH01000042.1 | 2 | CRS76791.1 | 13703 |
| 185 | GCA_001178345.1 | 148942 | 1336 | Streptococcus equi subsp. equi | EQUI0218 | Firmicutes | CVYI01000043.1 | 2 | CRS78515.1 | 13705 |
| 186 | GCA_001177385.1 | 148942 | 1336 | Streptococcus equi subsp. equi | EQUI0219 | Firmicutes | CVYJ01000068.1 | 2 | CRS90898.1 | 13707 |
| 187 | GCA_001179045.1 | 148942 | 1336 | Streptococcus equi subsp. equi | EQUI0221 | Firmicutes | CVYK01000065.1 | 2 | CRS79977.1 | 13709 |
| 188 | GCA_001178545.1 | 148942 | 1336 | Streptococcus equi subsp. equi | EQUI0224 | Firmicutes | CVYL01000063.1 | 2 | CRS84436.1 | 13711 |
| 189 | GCA_001179445.1 | 148942 | 1336 | Streptococcus equi subsp. equi | EQUI0223 | Firmicutes | CVYM01000062.1 | 2 | CRS81776.1 | 13713 |
| 190 | GCA_001178925.1 | 148942 | 1336 | Streptococcus equi subsp. equi | EQUI0049 | Firmicutes | CVYN01000043.1 | 2 | CRS82433.1 | 13715 |
| 191 | GCA_001177665.1 | 148942 | 1336 | Streptococcus equi subsp. equi | EQUI0234 | Firmicutes | CVYO01000041.1 | 2 | CRS93257.1 | 13717 |
| 192 | GCA_001175485.1 | 148942 | 1336 | Streptococcus equi subsp. equi | EQUI0226 | Firmicutes | CVYP01000057.1 | 2 | CRT34694.1 | 13719 |
| 193 | GCA_001178205.1 | 148942 | 1336 | Streptococcus equi subsp. equi | EQUI0237 | Firmicutes | CVYQ01000063.1 | 2 | CRT26926.1 | 13721 |
| 194 | GCA_001178065.1 | 148942 | 1336 | Streptococcus equi subsp. equi | EQUI0236 | Firmicutes | CVYR01000064.1 | 2 | CRT43878.1 | 13723 |
| 195 | GCA_001177345.1 | 148942 | 1336 | Streptococcus equi subsp. equi | EQUI0232 | Firmicutes | CVYS01000043.1 | 2 | CRT34280.1 | 13725 |
| 196 | GCA_001177405.1 | 148942 | 1336 | Streptococcus equi subsp. equi | EQUI0233 | Firmicutes | CVYT01000065.1 | 2 | CRT48070.1 | 13727 |
| 197 | GCA_001176725.1 | 148942 | 1336 | Streptococcus equi subsp. equi | EQUI0230 | Firmicutes | CVYU01000041.1 | 2 | CRT42031.1 | 13729 |
| 198 | GCA_001175145.1 | 148942 | 1336 | Streptococcus equi subsp. equi | EQUI0239 | Firmicutes | CVYV01000040.1 | 2 | CRT45621.1 | 13731 |
| 199 | GCA_001176105.1 | 148942 | 1336 | Streptococcus equi subsp. equi | EQUI0060 | Firmicutes | CVYW01000044.1 | 2 | CRT37702.1 | 13733 |
| 200 | GCA_001177885.1 | 148942 | 1336 | Streptococcus equi subsp. equi | EQUI0235 | Firmicutes | CVYX01000041.1 | 2 | CRT44937.1 | 13735 |
| 201 | GCA_001176125.1 | 148942 | 1336 | Streptococcus equi subsp. equi | EQUI0228 | Firmicutes | CVYY01000066.1 | 2 | CRT51318.1 | 13737 |
| 202 | GCA_001175185.1 | 148942 | 1336 | Streptococcus equi subsp. equi | EQUI0059 | Firmicutes | CVYZ01000043.1 | 2 | CRT46051.1 | 13739 |
| 203 | GCA_001178845.1 | 148942 | 1336 | Streptococcus equi subsp. equi | EQUI0225 | Firmicutes | CVZA01000065.1 | 2 | CRT46015.1 | 13741 |
| 204 | GCA_001176025.1 | 148942 | 1336 | Streptococcus equi subsp. equi | EQUI0238 | Firmicutes | CVZB01000068.1 | 2 | CRT64519.1 | 13743 |
| 205 | GCA_001175885.1 | 148942 | 1336 | Streptococcus equi subsp. equi | EQUI0062 | Firmicutes | CVZC01000026.1 | 2 | CRT20938.1 | 13745 |
| 206 | GCA_001175665.1 | 148942 | 1336 | Streptococcus equi subsp. equi | EQUI0061 | Firmicutes | CVZD01000042.1 | 2 | CRT55748.1 | 13747 |
| 207 | GCA_001178425.1 | 148942 | 1336 | Streptococcus equi subsp. equi | EQUI0063 | Firmicutes | CVZE01000061.1 | 2 | CRT61683.1 | 13749 |
| 208 | GCA_001179245.1 | 148942 | 1336 | Streptococcus equi subsp. equi | HO51380626 | Firmicutes | CVZF01000040.1 | 2 | CRT39243.1 | 13751 |
| 209 | GCA_001175405.1 | 148942 | 1336 | Streptococcus equi subsp. equi | EQUI0240 | Firmicutes | CVZG01000039.1 | 2 | CRT66749.1 | 13753 |
| 210 | GCA_001176385.1 | 148942 | 1336 | Streptococcus equi subsp. equi | EQUI0229 | Firmicutes | CVZH01000065.1 | 2 | CRT72248.1 | 13755 |
| 211 | GCA_001178805.1 | 148942 | 1336 | Streptococcus equi subsp. equi | EQUI0064 | Firmicutes | CVZI01000059.1 | 2 | CRT58939.1 | 13757 |
| 212 | GCA_001179125.1 | 148942 | 1336 | Streptococcus equi subsp. equi | EQUI0065 | Firmicutes | CVZJ01000061.1 | 2 | CRT49564.1 | 13759 |
| 213 | GCA_001175225.1 | 148942 | 1336 | Streptococcus equi subsp. equi | EQUI0227 | Firmicutes | CVZK01000068.1 | 2 | CRT72921.1 | 13761 |
| 214 | GCA_001176925.1 | 148942 | 1336 | Streptococcus equi subsp. equi | EQUI0231 | Firmicutes | CVZL01000044.1 | 2 | CRT74944.1 | 13763 |
| 215 | GCA_001179345.1 | 148942 | 1336 | Streptococcus equi subsp. equi | EQUI0067 | Firmicutes | CVZM01000061.1 | 2 | CRT61440.1 | 13765 |
| 216 | GCA_001176525.1 | 148942 | 1336 | Streptococcus equi subsp. equi | EQUI0068 | Firmicutes | CVZN01000058.1 | 2 | CRT76644.1 | 13767 |
| 217 | GCA_001178305.1 | 148942 | 1336 | Streptococcus equi subsp. equi | EQUI0071 | Firmicutes | CVZO01000056.1 | 2 | CRT71332.1 | 13769 |
| 218 | GCA_001176765.1 | 148942 | 1336 | Streptococcus equi subsp. equi | EQUI0069 | Firmicutes | CVZP01000060.1 | 2 | CRT68258.1 | 13771 |
| 219 | GCA_001178085.1 | 148942 | 1336 | Streptococcus equi subsp. equi | EQUI0070 | Firmicutes | CVZQ01000059.1 | 2 | CRT74820.1 | 13773 |
| 220 | GCA_001177605.1 | 148942 | 1336 | Streptococcus equi subsp. equi | EQUI0072 | Firmicutes | CVZR01000059.1 | 2 | CRT73973.1 | 13775 |
| 221 | GCA_001179065.1 | 148942 | 1336 | Streptococcus equi subsp. equi | EQUI0075 | Firmicutes | CVZS01000059.1 | 2 | CRT75021.1 | 13777 |
| 222 | GCA_001179405.1 | 148942 | 1336 | Streptococcus equi subsp. equi | EQUI0076 | Firmicutes | CVZT01000062.1 | 2 | CRT66743.1 | 13779 |
| 223 | GCA_001177545.1 | 148942 | 1336 | Streptococcus equi subsp. equi | EQUI0053 | Firmicutes | CVZU01000044.1 | 2 | CRT70587.1 | 13781 |
| 224 | GCA_001177965.1 | 148942 | 1336 | Streptococcus equi subsp. equi | EQUI0052 | Firmicutes | CVZV01000039.1 | 2 | CRT64134.1 | 13783 |
| 225 | GCA_001177065.1 | 148942 | 1336 | Streptococcus equi subsp. equi | EQUI0058 | Firmicutes | CVZW01000041.1 | 2 | CRT58016.1 | 13785 |
| 226 | GCA_001178565.1 | 148942 | 1336 | Streptococcus equi subsp. equi | EQUI0077 | Firmicutes | CVZX01000060.1 | 2 | CRT66308.1 | 13787 |
| 227 | GCA_001177485.1 | 148942 | 1336 | Streptococcus equi subsp. equi | EQUI0074 | Firmicutes | CVZY01000060.1 | 2 | CRT75771.1 | 13789 |
| 228 | GCA_001177865.1 | 148942 | 1336 | Streptococcus equi subsp. equi | EQUI0054 | Firmicutes | CWAA01000064.1 | 2 | CRT69364.1 | 13791 |
| 229 | GCA_001176905.1 | 148942 | 1336 | Streptococcus equi subsp. equi | EQUI0055 | Firmicutes | CWAB01000063.1 | 2 | CRT79535.1 | 13793 |
| 230 | GCA_001178105.1 | 148942 | 1336 | Streptococcus equi subsp. equi | EQUI0050 | Firmicutes | CWAC01000043.1 | 2 | CRT65384.1 | 13795 |
| 231 | GCA_001175245.1 | 148942 | 1336 | Streptococcus equi subsp. equi | EQUI0078 | Firmicutes | CWAD01000060.1 | 2 | CRT80463.1 | 13797 |
| 232 | GCA_001175925.1 | 148942 | 1336 | Streptococcus equi subsp. equi | EQUI0080 | Firmicutes | CWAE01000060.1 | 2 | CRT86531.1 | 13799 |
| 233 | GCA_001176325.1 | 148942 | 1336 | Streptococcus equi subsp. equi | EQUI0082 | Firmicutes | CWAF01000061.1 | 2 | CRT83007.1 | 13801 |
| 234 | GCA_001175985.1 | 148942 | 1336 | Streptococcus equi subsp. equi | EQUI0081 | Firmicutes | CWAG01000061.1 | 2 | CRT86803.1 | 13803 |
| 235 | GCA_001176605.1 | 148942 | 1336 | Streptococcus equi subsp. equi | EQUI0083 | Firmicutes | CWAH01000060.1 | 2 | CRT85044.1 | 13805 |
| 236 | GCA_001177025.1 | 148942 | 1336 | Streptococcus equi subsp. equi | EQUI0085 | Firmicutes | CWAI01000060.1 | 2 | CRT84205.1 | 13807 |
| 237 | GCA_001177285.1 | 148942 | 1336 | Streptococcus equi subsp. equi | EQUI0086 | Firmicutes | CWAJ01000043.1 | 2 | CRT81920.1 | 13809 |
| 238 | GCA_001176785.1 | 148942 | 1336 | Streptococcus equi subsp. equi | EQUI0084 | Firmicutes | CWAK01000064.1 | 2 | CRT86927.1 | 13811 |
| 239 | GCA_001177625.1 | 148942 | 1336 | Streptococcus equi subsp. equi | EQUI0087 | Firmicutes | CWAL01000044.1 | 2 | CRT82847.1 | 13813 |
| 240 | GCA_001175285.1 | 148942 | 1336 | Streptococcus equi subsp. equi | EQUI0088 | Firmicutes | CWAM01000044.1 | 2 | CRT86345.1 | 13815 |
| 241 | GCA_001175545.1 | 148942 | 1336 | Streptococcus equi subsp. equi | EQUI0079 | Firmicutes | CWAN01000059.1 | 2 | CRT89319.1 | 13817 |
| 242 | GCA_001175525.1 | 148942 | 1336 | Streptococcus equi subsp. equi | EQUI0089 | Firmicutes | CWAO01000054.1 | 2 | CRT87540.1 | 13819 |
| 243 | GCA_001175805.1 | 148942 | 1336 | Streptococcus equi subsp. equi | EQUI0090 | Firmicutes | CWAP01000063.1 | 2 | CRT90533.1 | 13821 |
| 244 | GCA_001176065.1 | 148942 | 1336 | Streptococcus equi subsp. equi | EQUI0092 | Firmicutes | CWAQ01000063.1 | 2 | CRT91761.1 | 13823 |
| 245 | GCA_001175625.1 | 148942 | 1336 | Streptococcus equi subsp. equi | EQUI0091 | Firmicutes | CWAR01000063.1 | 2 | CRU00009.1 | 13825 |
| 246 | GCA_001176265.1 | 148942 | 1336 | Streptococcus equi subsp. equi | EQUI0094 | Firmicutes | CWAS01000062.1 | 2 | CRT98477.1 | 13827 |
| 247 | GCA_001176945.1 | 148942 | 1336 | Streptococcus equi subsp. equi | EQUI0095 | Firmicutes | CWAT01000059.1 | 2 | CRT99301.1 | 13829 |
| 248 | GCA_001177265.1 | 148942 | 1336 | Streptococcus equi subsp. equi | EQUI0096 | Firmicutes | CWAU01000061.1 | 2 | CRT99600.1 | 13831 |
| 249 | GCA_001177225.1 | 148942 | 1336 | Streptococcus equi subsp. equi | EQUI0097 | Firmicutes | CWAV01000039.1 | 2 | CRU04445.1 | 13833 |
| 250 | GCA_001178005.1 | 148942 | 1336 | Streptococcus equi subsp. equi | EQUI0099 | Firmicutes | CWAW01000041.1 | 2 | CRU03267.1 | 13835 |
| 251 | GCA_001178145.1 | 148942 | 1336 | Streptococcus equi subsp. equi | EQUI0098 | Firmicutes | CWAX01000042.1 | 2 | CRU09874.1 | 13837 |
| 252 | GCA_001178785.1 | 148942 | 1336 | Streptococcus equi subsp. equi | EQUI0100 | Firmicutes | CWAY01000043.1 | 2 | CRU07135.1 | 13839 |
| 253 | GCA_001179105.1 | 148942 | 1336 | Streptococcus equi subsp. equi | EQUI0105 | Firmicutes | CWAZ01000043.1 | 2 | CRU20234.1 | 13841 |
| 254 | GCA_001178385.1 | 148942 | 1336 | Streptococcus equi subsp. equi | EQUI0106 | Firmicutes | CWBA01000062.1 | 2 | CRU18218.1 | 13843 |
| 255 | GCA_001178365.1 | 148942 | 1336 | Streptococcus equi subsp. equi | EQUI0103 | Firmicutes | CWBB01000068.1 | 2 | CRU22358.1 | 13845 |
| 256 | GCA_001179205.1 | 148942 | 1336 | Streptococcus equi subsp. equi | EQUI0107 | Firmicutes | CWBC01000062.1 | 2 | CRU18024.1 | 13847 |
| 257 | GCA_001177725.1 | 148942 | 1336 | Streptococcus equi subsp. equi | EQUI0108 | Firmicutes | CWBD01000063.1 | 2 | CRU21807.1 | 13849 |
| 258 | GCA_001177805.1 | 148942 | 1336 | Streptococcus equi subsp. equi | EQUI0109 | Firmicutes | CWBE01000063.1 | 2 | CRU20295.1 | 13851 |
| 259 | GCA_001177985.1 | 148942 | 1336 | Streptococcus equi subsp. equi | EQUI0110 | Firmicutes | CWBF01000047.1 | 2 | CRU20891.1 | 13853 |
| 260 | GCA_001176645.1 | 148942 | 1336 | Streptococcus equi subsp. equi | EQUI0114 | Firmicutes | CWBG01000041.1 | 2 | CRU31596.1 | 13855 |
| 261 | GCA_001176425.1 | 148942 | 1336 | Streptococcus equi subsp. equi | EQUI0112 | Firmicutes | CWBH01000044.1 | 2 | CRU58975.1 | 13857 |
| 262 | GCA_001176365.1 | 148942 | 1336 | Streptococcus equi subsp. equi | EQUI0113 | Firmicutes | CWBI01000040.1 | 2 | CRU85987.1 | 13859 |
| 263 | GCA_001178185.1 | 148942 | 1336 | Streptococcus equi subsp. equi | EQUI0111 | Firmicutes | CWBJ01000064.1 | 2 | CRU70022.1 | 13861 |
| 264 | GCA_900004825.1 | 148942 | 1336 | Streptococcus equi subsp. equi | EQUI0117 | Firmicutes | CWBK01000065.1 | 2 | CRU80198.1 | 13863 |
| 265 | GCA_001175085.1 | 148942 | 1336 | Streptococcus equi subsp. equi | EQUI0119 | Firmicutes | CWBL01000061.1 | 2 | CRU71456.1 | 13865 |
| 266 | GCA_001176845.1 | 148942 | 1336 | Streptococcus equi subsp. equi | EQUI0115 | Firmicutes | CWBM01000065.1 | 2 | CRU70674.1 | 13867 |
| 267 | GCA_001175425.1 | 148942 | 1336 | Streptococcus equi subsp. equi | EQUI0118 | Firmicutes | CWBN01000062.1 | 2 | CRU61491.1 | 13869 |
| 268 | GCA_001175505.1 | 148942 | 1336 | Streptococcus equi subsp. equi | EQUI0128 | Firmicutes | CWBO01000044.1 | 2 | CRU48069.1 | 13871 |
| 269 | GCA_001178225.1 | 148942 | 1336 | Streptococcus equi subsp. equi | EQUI0126 | Firmicutes | CWBP01000061.1 | 2 | CRU68120.1 | 13873 |
| 270 | GCA_001175325.1 | 148942 | 1336 | Streptococcus equi subsp. equi | EQUI0129 | Firmicutes | CWBQ01000061.1 | 2 | CRU71296.1 | 13875 |
| 271 | GCA_001179025.1 | 148942 | 1336 | Streptococcus equi subsp. equi | EQUI0120 | Firmicutes | CWBR01000065.1 | 2 | CRU93798.1 | 13877 |
| 272 | GCA_001176145.1 | 148942 | 1336 | Streptococcus equi subsp. equi | EQUI0130 | Firmicutes | CWBS01000043.1 | 2 | CRU91202.1 | 13879 |
| 273 | GCA_001177505.1 | 148942 | 1336 | Streptococcus equi subsp. equi | EQUI0127 | Firmicutes | CWBT01000042.1 | 2 | CRU91151.1 | 13881 |
| 274 | GCA_001178045.1 | 148942 | 1336 | Streptococcus equi subsp. equi | EQUI0125 | Firmicutes | CWBU01000045.1 | 2 | CRU94321.1 | 13883 |
| 275 | GCA_001178825.1 | 148942 | 1336 | Streptococcus equi subsp. equi | EQUI0123 | Firmicutes | CWBV01000042.1 | 2 | CRU82344.1 | 13885 |
| 276 | GCA_001178465.1 | 148942 | 1336 | Streptococcus equi subsp. equi | EQUI0122 | Firmicutes | CWBW01000043.1 | 2 | CRU80816.1 | 13887 |
| 277 | GCA_001175725.1 | 148942 | 1336 | Streptococcus equi subsp. equi | EQUI0131 | Firmicutes | CWBX01000064.1 | 2 | CRU91403.1 | 13889 |
| 278 | GCA_001179225.1 | 148942 | 1336 | Streptococcus equi subsp. equi | EQUI0121 | Firmicutes | CWBY01000040.1 | 2 | CRU86635.1 | 13891 |
| 279 | GCA_001177145.1 | 148942 | 1336 | Streptococcus equi subsp. equi | EQUI0134 | Firmicutes | CWBZ01000059.1 | 2 | CRU82566.1 | 13893 |
| 280 | GCA_001177045.1 | 148942 | 1336 | Streptococcus equi subsp. equi | EQUI0136 | Firmicutes | CWCA01000061.1 | 2 | CRU96729.1 | 13895 |
| 281 | GCA_001177465.1 | 148942 | 1336 | Streptococcus equi subsp. equi | EQUI0139 | Firmicutes | CWCB01000061.1 | 2 | CRU83052.1 | 13897 |
| 282 | GCA_001179305.1 | 148942 | 1336 | Streptococcus equi subsp. equi | EQUI0140 | Firmicutes | CWCC01000060.1 | 2 | CRU80987.1 | 13899 |
| 283 | GCA_001178965.1 | 148942 | 1336 | Streptococcus equi subsp. equi | EQUI0141 | Firmicutes | CWCD01000060.1 | 2 | CRU78242.1 | 13901 |
| 284 | GCA_001176665.1 | 148942 | 1336 | Streptococcus equi subsp. equi | EQUI0132 | Firmicutes | CWCE01000060.1 | 2 | CRV00464.1 | 13903 |
| 285 | GCA_001178865.1 | 148942 | 1336 | Streptococcus equi subsp. equi | EQUI0124 | Firmicutes | CWCF01000043.1 | 2 | CRV01108.1 | 13905 |
| 286 | GCA_001177425.1 | 148942 | 1336 | Streptococcus equi subsp. equi | EQUI0137 | Firmicutes | CWCG01000042.1 | 2 | CRU80203.1 | 13907 |
| 287 | GCA_001178945.1 | 148942 | 1336 | Streptococcus equi subsp. equi | EQUI0142 | Firmicutes | CWCH01000042.1 | 2 | CRU93107.1 | 13909 |
| 288 | GCA_001177305.1 | 148942 | 1336 | Streptococcus equi subsp. equi | EQUI0135 | Firmicutes | CWCI01000040.1 | 2 | CRU95695.1 | 13911 |
| 289 | GCA_001178605.1 | 148942 | 1336 | Streptococcus equi subsp. equi | EQUI0143 | Firmicutes | CWCJ01000060.1 | 2 | CRU99763.1 | 13913 |
| 290 | GCA_001177685.1 | 148942 | 1336 | Streptococcus equi subsp. equi | EQUI0138 | Firmicutes | CWCK01000040.1 | 2 | CRV05021.1 | 13915 |
| 291 | GCA_001177125.1 | 148942 | 1336 | Streptococcus equi subsp. equi | EQUI0155 | Firmicutes | CWCL01000043.1 | 2 | CRU97029.1 | 13917 |
| 292 | GCA_001175205.1 | 148942 | 1336 | Streptococcus equi subsp. equi | EQUI0156 | Firmicutes | CWCM01000063.1 | 2 | CRV01704.1 | 13919 |
| 293 | GCA_001175265.1 | 148942 | 1336 | Streptococcus equi subsp. equi | EQUI0157 | Firmicutes | CWCN01000060.1 | 2 | CRV11871.1 | 13921 |
| 294 | GCA_001175605.1 | 148942 | 1336 | Streptococcus equi subsp. equi | EQUI0158 | Firmicutes | CWCO01000063.1 | 2 | CRV20248.1 | 13923 |
| 295 | GCA_001178525.1 | 148942 | 1336 | Streptococcus equi subsp. equi | EQUI0160 | Firmicutes | CWCP01000061.1 | 2 | CRV16484.1 | 13925 |
| 296 | GCA_001176005.1 | 148942 | 1336 | Streptococcus equi subsp. equi | EQUI0145 | Firmicutes | CWCQ01000042.1 | 2 | CRV16534.1 | 13927 |
| 297 | GCA_001178885.1 | 148942 | 1336 | Streptococcus equi subsp. equi | EQUI0159 | Firmicutes | CWCR01000064.1 | 2 | CRV08843.1 | 13925 |
| 298 | GCA_001176345.1 | 148942 | 1336 | Streptococcus equi subsp. equi | EQUI0133 | Firmicutes | CWCS01000043.1 | 2 | CRV21833.1 | 13931 |
| 299 | GCA_001178505.1 | 148942 | 1336 | Streptococcus equi subsp. equi | EQUI0161 | Firmicutes | CWCT01000071.1 | 2 | CRV22383.1 | 13933 |
| 300 | GCA_001177905.1 | 148942 | 1336 | Streptococcus equi subsp. equi | EQUI0164 | Firmicutes | CWCU01000062.1 | 2 | CRV15611.1 | 13935 |
| 301 | GCA_001179005.1 | 148942 | 1336 | Streptococcus equi subsp. equi | EQUI0163 | Firmicutes | CWCV01000063.1 | 2 | CRV21784.1 | 13937 |
| 302 | GCA_001179285.1 | 148942 | 1336 | Streptococcus equi subsp. equi | EQUI0162 | Firmicutes | CWCW01000068.1 | 2 | CRV18917.1 | 13935 |
| 303 | GCA_001175785.1 | 148942 | 1336 | Streptococcus equi subsp. equi | EQUI0146 | Firmicutes | CWCX01000062.1 | 2 | CRV22810.1 | 13941 |
| 304 | GCA_001178325.1 | 148942 | 1336 | Streptococcus equi subsp. equi | EQUI0166 | Firmicutes | CWCY01000063.1 | 2 | CRV26120.1 | 13943 |
| 305 | GCA_001177565.1 | 148942 | 1336 | Streptococcus equi subsp. equi | EQUI0165 | Firmicutes | CWCZ01000062.1 | 2 | CRV22947.1 | 13945 |
| 306 | GCA_001178125.1 | 148942 | 1336 | Streptococcus equi subsp. equi | EQUI0167 | Firmicutes | CWDA01000061.1 | 2 | CRV22945.1 | 13947 |
| 307 | GCA_001177005.1 | 148942 | 1336 | Streptococcus equi subsp. equi | EQUI0168 | Firmicutes | CWDB01000062.1 | 2 | CRV23365.1 | 13945 |
| 308 | GCA_001176625.1 | 148942 | 1336 | Streptococcus equi subsp. equi | EQUI0169 | Firmicutes | CWDC01000065.1 | 2 | CRV25535.1 | 13951 |
| 309 | GCA_001175565.1 | 148942 | 1336 | Streptococcus equi subsp. equi | EQUI0147 | Firmicutes | CWDD01000044.1 | 2 | CRV23988.1 | 13953 |
| 310 | GCA_001177185.1 | 148942 | 1336 | Streptococcus equi subsp. equi | EQUI0148 | Firmicutes | CWDE01000094.1 | 2 | CRV35426.1 | 13955 |
| 311 | GCA_001176305.1 | 148942 | 1336 | Streptococcus equi subsp. equi | EQUI0149 | Firmicutes | CWDF01000064.1 | 2 | CRV28496.1 | 13957 |
| 312 | GCA_001176545.1 | 148942 | 1336 | Streptococcus equi subsp. equi | EQUI0150 | Firmicutes | CWDG01000061.1 | 2 | CRV30744.1 | 13955 |
| 313 | GCA_001176445.1 | 148942 | 1336 | Streptococcus equi subsp. equi | EQUI0152 | Firmicutes | CWDH01000059.1 | 2 | CRV37963.1 | 13961 |
| 314 | GCA_001176805.1 | 148942 | 1336 | Streptococcus equi subsp. equi | EQUI0151 | Firmicutes | CWDI01000059.1 | 2 | CRV37002.1 | 13963 |
| 315 | GCA_001176865.1 | 148942 | 1336 | Streptococcus equi subsp. equi | EQUI0154 | Firmicutes | CWDJ01000042.1 | 2 | CRV38823.1 | 13965 |
| 316 | GCA_001176685.1 | 148942 | 1336 | Streptococcus equi subsp. equi | EQUI0153 | Firmicutes | CWDK01000064.1 | 2 | CRV39782.1 | 13967 |
| 317 | GCA_001176245.1 | 148942 | 1336 | Streptococcus equi subsp. equi | EQUI0170 | Firmicutes | CWDL01000040.1 | 2 | CRV45924.1 | 13965 |
| 318 | GCA_001175945.1 | 148942 | 1336 | Streptococcus equi subsp. equi | EQUI0171 | Firmicutes | CWDM01000043.1 | 2 | CRV48900.1 | 13971 |
| 319 | GCA_001176225.1 | 148942 | 1336 | Streptococcus equi subsp. equi | EQUI0172 | Firmicutes | CWDN01000043.1 | 2 | CRV53138.1 | 13973 |
| 320 | GCA_001175465.1 | 148942 | 1336 | Streptococcus equi subsp. equi | EQUI0174 | Firmicutes | CWDO01000043.1 | 2 | CRV61840.1 | 13975 |
| 321 | GCA_001175825.1 | 148942 | 1336 | Streptococcus equi subsp. equi | EQUI0173 | Firmicutes | CWDP01000041.1 | 2 | CRV60058.1 | 13977 |
| 322 | GCA_001175125.1 | 148942 | 1336 | Streptococcus equi subsp. equi | EQUI0175 | Firmicutes | CWDQ01000043.1 | 2 | CRV55434.1 | 13975 |
| 323 | GCA_001178265.1 | 148942 | 1336 | Streptococcus equi subsp. equi | EQUI0178 | Firmicutes | CWDR01000044.1 | 2 | CRV60803.1 | 13981 |
| 324 | GCA_001178665.1 | 148942 | 1336 | Streptococcus equi subsp. equi | EQUI0179 | Firmicutes | CWDS01000044.1 | 2 | CRW03962.1 | 13983 |
| 325 | GCA_001179365.1 | 148942 | 1336 | Streptococcus equi subsp. equi | EQUI0176 | Firmicutes | CWDT01000009.1 | 2 | CRV54211.1 | 13985 |
| 326 | GCA_001179165.1 | 148942 | 1336 | Streptococcus equi subsp. equi | EQUI0177 | Firmicutes | CWDU01000042.1 | 2 | CRV83270.1 | 13987 |
| 327 | GCA_001179385.1 | 148942 | 1336 | Streptococcus equi subsp. equi | EQUI0183 | Firmicutes | CWDV01000041.1 | 2 | CRV91455.1 | 13985 |
| 328 | GCA_001175445.1 | 148942 | 1336 | Streptococcus equi subsp. equi | EQUI0184 | Firmicutes | CWDW01000042.1 | 2 | CRV92191.1 | 13991 |
| 329 | GCA_001177165.1 | 148942 | 1336 | Streptococcus equi subsp. equi | EQUI0195 | Firmicutes | CWDX01000062.1 | 2 | CRV87136.1 | 13993 |
| 330 | GCA_001177325.1 | 148942 | 1336 | Streptococcus equi subsp. equi | EQUI0196 | Firmicutes | CWDY01000065.1 | 2 | CRV84626.1 | 13995 |
| 331 | GCA_001178985.1 | 148942 | 1336 | Streptococcus equi subsp. equi | EQUI0181 | Firmicutes | CWDZ01000042.1 | 2 | CRW02988.1 | 13997 |
| 332 | GCA_001178725.1 | 148942 | 1336 | Streptococcus equi subsp. equi | EQUI0180 | Firmicutes | CWEA01000042.1 | 2 | CRV97209.1 | 13995 |
| 333 | GCA_001177765.1 | 148942 | 1336 | Streptococcus equi subsp. equi | EQUI0192 | Firmicutes | CWEB01000061.1 | 2 | CRW00524.1 | 14001 |
| 334 | GCA_001179265.1 | 148942 | 1336 | Streptococcus equi subsp. equi | EQUI0182 | Firmicutes | CWEC01000045.1 | 2 | CRW01316.1 | 14003 |
| 335 | GCA_001178285.1 | 148942 | 1336 | Streptococcus equi subsp. equi | EQUI0191 | Firmicutes | CWED01000041.1 | 2 | CRV92086.1 | 14005 |
| 336 | GCA_001177705.1 | 148942 | 1336 | Streptococcus equi subsp. equi | EQUI0194 | Firmicutes | CWEE01000065.1 | 2 | CRV95671.1 | 14007 |
| 337 | GCA_001176085.1 | 148942 | 1336 | Streptococcus equi subsp. equi | EQUI0199 | Firmicutes | CWEF01000062.1 | 2 | CRV97947.1 | 14005 |
| 338 | GCA_001175045.1 | 148942 | 1336 | Streptococcus equi subsp. equi | EQUI0185 | Firmicutes | CWEG01000008.1 | 2 | CRV78527.1 | 14011 |
| 339 | GCA_001178645.1 | 148942 | 1336 | Streptococcus equi subsp. equi | EQUI0188 | Firmicutes | CWEH01000043.1 | 2 | CRW03687.1 | 14013 |
| 340 | GCA_001175025.1 | 148942 | 1336 | Streptococcus equi subsp. equi | EQUI0198 | Firmicutes | CWEI01000062.1 | 2 | CRV92165.1 | 14015 |
| 341 | GCA_001176465.1 | 148942 | 1336 | Streptococcus equi subsp. equi | EQUI0197 | Firmicutes | CWEJ01000060.1 | 2 | CRV95850.1 | 14017 |
| 342 | GCA_001175685.1 | 148942 | 1336 | Streptococcus equi subsp. equi | EQUI0200 | Firmicutes | CWEK01000061.1 | 2 | CRV90197.1 | 14015 |
| 343 | GCA_001176205.1 | 148942 | 1336 | Streptococcus equi subsp. equi | EQUI0186 | Firmicutes | CWEL01000046.1 | 2 | CRW02430.1 | 14021 |
| 344 | GCA_001175905.1 | 148942 | 1336 | Streptococcus equi subsp. equi | EQUI0187 | Firmicutes | CWEM01000042.1 | 2 | CRW06361.1 | 14023 |
| 345 | GCA_001175745.1 | 148942 | 1336 | Streptococcus equi subsp. equi | EQUI0201 | Firmicutes | CWEN01000063.1 | 2 | CRW10290.1 | 14025 |
| 346 | GCA_001176565.1 | 148942 | 1336 | Streptococcus equi subsp. equi | EQUI0012 | Firmicutes | CWEP01000057.1 | 2 | CRV93329.1 | 14027 |
| 347 | GCA_001178445.1 | 148942 | 1336 | Streptococcus equi subsp. equi | EQUI0203 | Firmicutes | CWEQ01000063.1 | 2 | CRW10487.1 | 14029 |
| 348 | GCA_001178705.1 | 148942 | 1336 | Streptococcus equi subsp. equi | EQUI0202 | Firmicutes | CWES01000061.1 | 2 | CRW11899.1 | 14031 |
| 349 | GCA_001179465.1 | 148942 | 1336 | Streptococcus equi subsp. equi | EQUI0205 | Firmicutes | CWEV01000037.1 | 2 | CRW12157.1 | 14033 |
| 350 | GCA_001178245.1 | 148942 | 1336 | Streptococcus equi subsp. equi | EQUI0208 | Firmicutes | CWEW01000061.1 | 2 | CRW18036.1 | 14035 |
| 351 | GCA_001177925.1 | 148942 | 1336 | Streptococcus equi subsp. equi | EQUI0206 | Firmicutes | CWEX01000064.1 | 2 | CRW22900.1 | 14037 |
| 352 | GCA_001177745.1 | 148942 | 1336 | Streptococcus equi subsp. equi | EQUI0207 | Firmicutes | CWEY01000063.1 | 2 | CRW16828.1 | 14039 |
| 353 | GCA_001176185.1 | 148942 | 1336 | Streptococcus equi subsp. equi | EQUI0209 | Firmicutes | CWEZ01000060.1 | 2 | CRW17377.1 | 14041 |
| 354 | GCA_001175865.1 | 148942 | 1336 | Streptococcus equi subsp. equi | EQUI0211 | Firmicutes | CWFA01000062.1 | 2 | CRW41572.1 | 14043 |
| 355 | GCA_001175385.1 | 148942 | 1336 | Streptococcus equi subsp. equi | EQUI0212 | Firmicutes | CWFB01000064.1 | 2 | CRW33338.1 | 14045 |
| 356 | GCA_001175165.1 | 148942 | 1336 | Streptococcus equi subsp. equi | EQUI0213 | Firmicutes | CWFC01000062.1 | 2 | CRW28598.1 | 14047 |
| 357 | GCA_001176745.1 | 148942 | 1336 | Streptococcus equi subsp. equi | EQUI0216 | Firmicutes | CWFD01000040.1 | 2 | CRW38404.1 | 14049 |
| 358 | GCA_001176405.1 | 148942 | 1336 | Streptococcus equi subsp. equi | EQUI0217 | Firmicutes | CWFE01000062.1 | 2 | CRW56664.1 | 14051 |
| 359 | GCA_001177085.1 | 148942 | 1336 | Streptococcus equi subsp. equi | EQUI0214 | Firmicutes | CWFF01000066.1 | 2 | CRW57488.1 | 14053 |
| 360 | GCA_001177525.1 | 148942 | 1336 | Streptococcus equi subsp. equi | EQUI0002 | Firmicutes | CWFG01000057.1 | 2 | CRW51150.1 | 14055 |
| 361 | GCA_001176985.1 | 148942 | 1336 | Streptococcus equi subsp. equi | EQUI0215 | Firmicutes | CWFI01000066.1 | 2 | CRW56037.1 | 14057 |
| 362 | GCA_001177365.1 | 148942 | 1336 | Streptococcus equi subsp. equi | EQUI0013 | Firmicutes | CWFJ01000060.1 | 2 | CRW51019.1 | 14059 |
| 363 | GCA_001177105.1 | 148942 | 1336 | Streptococcus equi subsp. equi | EQUI0014 | Firmicutes | CWFK01000062.1 | 2 | CRW62422.1 | 14061 |
| 364 | GCA_001176885.1 | 148942 | 1336 | Streptococcus equi subsp. equi | EQUI0015 | Firmicutes | CWFL01000059.1 | 2 | CRW52635.1 | 14063 |
| 365 | GCA_001177585.1 | 148942 | 1336 | Streptococcus equi subsp. equi | EQUI0016 | Firmicutes | CWFM01000059.1 | 2 | CRW57925.1 | 14065 |
| 366 | GCA_001177825.1 | 148942 | 1336 | Streptococcus equi subsp. equi | EQUI0017 | Firmicutes | CWFN01000059.1 | 2 | CRW58490.1 | 14067 |
| 367 | GCA_001177645.1 | 148942 | 1336 | Streptococcus equi subsp. equi | EQUI0018 | Firmicutes | CWFO01000060.1 | 2 | CRW59062.1 | 14069 |
| 368 | GCA_001179325.1 | 148942 | 1336 | Streptococcus equi subsp. equi | EQUI0019 | Firmicutes | CWFS01000065.1 | 2 | CRW78794.1 | 14071 |
| 369 | GCA_001179145.1 | 148942 | 1336 | Streptococcus equi subsp. equi | EQUI0020 | Firmicutes | CWFT01000065.1 | 2 | CRW72044.1 | 14073 |
| 370 | GCA_001178745.1 | 148942 | 1336 | Streptococcus equi subsp. equi | EQUI0021 | Firmicutes | CWFU01000062.1 | 2 | CRW79959.1 | 14075 |
| 371 | GCA_001178405.1 | 148942 | 1336 | Streptococcus equi subsp. equi | EQUI0022 | Firmicutes | CWFV01000063.1 | 2 | CRW76315.1 | 14077 |
| 372 | GCA_001176165.1 | 148942 | 1336 | Streptococcus equi subsp. equi | EQUI0023 | Firmicutes | CWFW01000065.1 | 2 | CRW80108.1 | 14079 |
| 373 | GCA_001175765.1 | 148942 | 1336 | Streptococcus equi subsp. equi | EQUI0024 | Firmicutes | CWFX01000059.1 | 2 | CRW66993.1 | 14081 |
| 374 | GCA_001175705.1 | 148942 | 1336 | Streptococcus equi subsp. equi | EQUI0025 | Firmicutes | CWFY01000062.1 | 2 | CRW70529.1 | 14083 |
| 375 | GCA_001175345.1 | 148942 | 1336 | Streptococcus equi subsp. equi | EQUI0026 | Firmicutes | CWFZ01000062.1 | 2 | CRW76738.1 | 14085 |
| 376 | GCA_001175065.1 | 148942 | 1336 | Streptococcus equi subsp. equi | EQUI0027 | Firmicutes | CWGA01000061.1 | 2 | CRW76864.1 | 14087 |
| 377 | GCA_001176485.1 | 148942 | 1336 | Streptococcus equi subsp. equi | EQUI0028 | Firmicutes | CWGB01000067.1 | 2 | CRW80973.1 | 14089 |
| 378 | GCA_001176285.1 | 148942 | 1336 | Streptococcus equi subsp. equi | EQUI0029 | Firmicutes | CWGC01000064.1 | 2 | CRW81695.1 | 14091 |
| 379 | GCA_001176585.1 | 148942 | 1336 | Streptococcus equi subsp. equi | EQUI0030 | Firmicutes | CWGD01000063.1 | 2 | CRW77653.1 | 14093 |
| 380 | GCA_001177945.1 | 148942 | 1336 | Streptococcus equi subsp. equi | EQUI0031 | Firmicutes | CWGE01000064.1 | 2 | CRW79780.1 | 14095 |
| 381 | GCA_001177445.1 | 148942 | 1336 | Streptococcus equi subsp. equi | EQUI0032 | Firmicutes | CWGF01000063.1 | 2 | CRW80948.1 | 14097 |
| 382 | GCA_001285185.1 | 562 | 562 | Escherichia coli | 102485_aEPEC | Proteobacteria | CYBC01000016.1 | 2 | CTT29869.1 | 14099 |
| 383 | GCA_001286125.1 | 562 | 562 | Escherichia coli | 302710 | Proteobacteria | CYFG01000018.1 | 2 | CTZ05351.1 | 14101 |
| 384 | GCA_001286985.1 | 1408 | 1408 | Bacillus pumilus | | Firmicutes | CYHK01000004.1 | 2 | CUB16899.1 | 14103 |
| 385 | GCA_001299055.1 | 1806504 | 1806504 | Achromobacter sp. 2789STDY5608606 | 2789STDY5608606 | Proteobacteria | CYTI01000001.1 | 5 | CUI41065.1 | 14105 |
| 386 | GCA_001298995.1 | 1806492 | 1806492 | Achromobacter sp. 2789STDY5608615 | 2789STDY5608615 | Proteobacteria | CYTY01000055.1 | 3 | CUK22707.1 | 14110 |
| 387 | GCA_001564615.1 | 1639 | 1639 | Listeria monocytogenes | LM06-00444 | Firmicutes | CYUX01000002.1 | 2 | CUK50734.1 | 14113 |
| 388 | GCA_001564675.1 | 1639 | 1639 | Listeria monocytogenes | LM06-00918 | Firmicutes | CYVA01000003.1 | 2 | CUK61280.1 | 14115 |
| 389 | GCA_001564895.1 | 1639 | 1639 | Listeria monocytogenes | LM07-01042 | Firmicutes | CYVK01000002.1 | 2 | CUK91820.1 | 14117 |
| 390 | GCA_000153885.1 | 411463 | 39496 | Eubacterium ventriosum ATCC 27560 | ATCC 27560 | Firmicutes | DS264285.1 | 4 | EDM51055.1 | 14119 |
| 391 | GCA_001537545.1 | 615 | 615 | Serratia marcescens | 2880STDY5682911 | Proteobacteria | FCFC01000005.1 | 2 | CUY08442.1 | 14123 |
| 392 | GCA_001538985.1 | 615 | 615 | Serratia marcescens | 2880STDY5683036 | Proteobacteria | FCFE01000005.1 | 2 | CUZ68635.1 | 14125 |
| 393 | GCA_001538605.1 | 615 | 615 | Serratia marcescens | 2880STDY5682822 | Proteobacteria | FCFF01000005.1 | 2 | CUZ82287.1 | 14127 |
| 394 | GCA_001537885.1 | 615 | 615 | Serratia marcescens | 2880STDY5682825 | Proteobacteria | FCFG01000007.1 | 2 | CUZ53146.1 | 14129 |
| 395 | GCA_001539305.1 | 615 | 615 | Serratia marcescens | 2880STDY5683007 | Proteobacteria | FCFH01000009.1 | 2 | CVA27382.1 | 14131 |
| 396 | GCA_001537405.1 | 615 | 615 | Serratia marcescens | 2880STDY5683035 | Proteobacteria | FCFI01000002.1 | 2 | CUZ41368.1 | 14133 |
| 397 | GCA_001540125.1 | 615 | 615 | Serratia marcescens | 2880STDY5683037 | Proteobacteria | FCFJ01000004.1 | 2 | CVA08047.1 | 14135 |
| 398 | GCA_001538905.1 | 615 | 615 | Serratia marcescens | 2880STDY5682814 | Proteobacteria | FCFK01000006.1 | 2 | CUZ42305.1 | 14137 |
| 399 | GCA_001537445.1 | 615 | 615 | Serratia marcescens | 2880STDY5683014 | Proteobacteria | FCFL01000006.1 | 2 | CVA28869.1 | 14139 |
| 400 | GCA_001539185.1 | 615 | 615 | Serratia marcescens | 2880STDY5683009 | Proteobacteria | FCFM01000003.1 | 2 | CUZ16125.1 | 14141 |
| 401 | GCA_001540805.1 | 615 | 615 | Serratia marcescens | 2880STDY5682837 | Proteobacteria | FCFN01000007.1 | 2 | CUZ60995.1 | 14143 |
| 402 | GCA_001538525.1 | 615 | 615 | Serratia marcescens | 2880STDY5682824 | Proteobacteria | FCFO01000009.1 | 2 | CUZ90935.1 | 14145 |
| 403 | GCA_001539805.1 | 615 | 615 | Serratia marcescens | 2880STDY5682834 | Proteobacteria | FCFP01000006.1 | 2 | CUZ73394.1 | 14147 |
| 404 | GCA_001538705.1 | 615 | 615 | Serratia marcescens | 2880STDY5683032 | Proteobacteria | FCFQ01000004.1 | 2 | CVA13553.1 | 14149 |
| 405 | GCA_001539125.1 | 615 | 615 | Serratia marcescens | 2880STDY5683011 | Proteobacteria | FCFR01000005.1 | 2 | CVA22943.1 | 14151 |
| 406 | GCA_001538465.1 | 615 | 615 | Serratia marcescens | 2880STDY5683010 | Proteobacteria | FCFS01000001.1 | 2 | CUY16546.1 | 14153 |
| 407 | GCA_001539505.1 | 615 | 615 | Serratia marcescens | 2880STDY5682831 | Proteobacteria | FCFT01000005.1 | 2 | CUZ58663.1 | 14155 |
| 408 | GCA_001538485.1 | 615 | 615 | Serratia marcescens | 2880STDY5683021 | Proteobacteria | FCFU01000011.1 | 2 | CUZ73629.1 | 14157 |
| 409 | GCA_001539785.1 | 615 | 615 | Serratia marcescens | 2880STDY5682827 | Proteobacteria | FCFV01000002.1 | 2 | CUZ84855.1 | 14159 |
| 410 | GCA_001537205.1 | 615 | 615 | Serratia marcescens | 2880STDY5682828 | Proteobacteria | FCFW01000002.1 | 2 | CUZ51222.1 | 14161 |
| 411 | GCA_001536485.1 | 615 | 615 | Serratia marcescens | 2880STDY5682815 | Proteobacteria | FCFX01000005.1 | 2 | CVA06032.1 | 14163 |
| 412 | GCA_001537765.1 | 615 | 615 | Serratia marcescens | 2880STDY5682820 | Proteobacteria | FCFY01000007.1 | 2 | CUZ66191.1 | 14165 |
| 413 | GCA_001537525.1 | 615 | 615 | Serratia marcescens | 2880STDY5682816 | Proteobacteria | FCFZ01000005.1 | 2 | CUZ96331.1 | 14167 |
| 414 | GCA_001540665.1 | 615 | 615 | Serratia marcescens | 2880STDY5683018 | Proteobacteria | FCGA01000006.1 | 2 | CUZ56035.1 | 14169 |
| 415 | GCA_001538625.1 | 615 | 615 | Serratia marcescens | 2880STDY5682836 | Proteobacteria | FCGB01000003.1 | 2 | CUZ47627.1 | 14171 |
| 416 | GCA_001537105.1 | 615 | 615 | Serratia marcescens | 2880STDY5683024 | Proteobacteria | FCGC01000005.1 | 2 | CUZ65513.1 | 14173 |
| 417 | GCA_001537145.1 | 615 | 615 | Serratia marcescens | 2880STDY5682819 | Proteobacteria | FCGD01000005.1 | 2 | CUZ64473.1 | 14175 |
| 418 | GCA_001539985.1 | 615 | 615 | Serratia marcescens | 2880STDY5683033 | Proteobacteria | FCGF01000007.1 | 2 | CUZ76085.1 | 14177 |
| 419 | GCA_001536605.1 | 615 | 615 | Serratia marcescens | 2880STDY5683026 | Proteobacteria | FCGG01000006.1 | 2 | CVA13444.1 | 14179 |
| 420 | GCA_001537845.1 | 615 | 615 | Serratia marcescens | 2880STDY5682823 | Proteobacteria | FCGH01000006.1 | 2 | CVA27078.1 | 14181 |
| 421 | GCA_001539225.1 | 615 | 615 | Serratia marcescens | 2880STDY5683013 | Proteobacteria | FCGI01000005.1 | 2 | CVA15880.1 | 14183 |
| 422 | GCA_001540225.1 | 615 | 615 | Serratia marcescens | 2880STDY5683031 | Proteobacteria | FCGJ01000005.1 | 2 | CUZ46954.1 | 14185 |
| 423 | GCA_001537565.1 | 615 | 615 | Serratia marcescens | 2880STDY5683034 | Proteobacteria | FCGK01000008.1 | 2 | CUZ31026.1 | 14187 |
| 424 | GCA_001539965.1 | 615 | 615 | Serratia marcescens | 2880STDY5682829 | Proteobacteria | FCGL01000002.1 | 2 | CUZ02629.1 | 14189 |
| 425 | GCA_001537645.1 | 615 | 615 | Serratia marcescens | 2880STDY5682830 | Proteobacteria | FCGM01000004.1 | 2 | CUZ51326.1 | 14191 |
| 426 | GCA_001539485.1 | 615 | 615 | Serratia marcescens | 2880STDY5683008 | Proteobacteria | FCGN01000006.1 | 2 | CUZ55076.1 | 14193 |
| 427 | GCA_001537285.1 | 615 | 615 | Serratia marcescens | 2880STDY5683022 | Proteobacteria | FCGO01000004.1 | 2 | CVA20194.1 | 14195 |
| 428 | GCA_001539645.1 | 615 | 615 | Serratia marcescens | 2880STDY5682838 | Proteobacteria | FCGP01000007.1 | 2 | CUZ69684.1 | 14197 |
| 429 | GCA_001538765.1 | 615 | 615 | Serratia marcescens | 2880STDY5682826 | Proteobacteria | FCGQ01000005.1 | 2 | CUZ82709.1 | 14199 |
| 430 | GCA_001537825.1 | 615 | 615 | Serratia marcescens | 2880STDY5683030 | Proteobacteria | FCGR01000003.1 | 2 | CUZ92205.1 | 14201 |
| 431 | GCA_001538195.1 | 615 | 615 | Serratia marcescens | 2880STDY5683020 | Proteobacteria | FCGS01000007.1 | 2 | CUZ80077.1 | 14203 |
| 432 | GCA_001540185.1 | 615 | 615 | Serratia marcescens | 2880STDY5682832 | Proteobacteria | FCGT01000002.1 | 2 | CUZ43216.1 | 14205 |
| 433 | GCA_001539025.1 | 615 | 615 | Serratia marcescens | 2880STDY5682818 | Proteobacteria | FCGU01000004.1 | 2 | CVA39165.1 | 14207 |
| 434 | GCA_001540205.1 | 615 | 615 | Serratia marcescens | 2880STDY5682842 | Proteobacteria | FCGV01000008.1 | 2 | CVA66719.1 | 14209 |
| 435 | GCA_001539725.1 | 615 | 615 | Serratia marcescens | 2880STDY5682839 | Proteobacteria | FCGW01000003.1 | 2 | CUZ78091.1 | 14211 |
| 436 | GCA_001537425.1 | 615 | 615 | Serratia marcescens | 2880STDY5682840 | Proteobacteria | FCGX01000008.1 | 2 | CUZ53769.1 | 14213 |
| 437 | GCA_001540725.1 | 615 | 615 | Serratia marcescens | 2880STDY5682844 | Proteobacteria | FCGY01000002.1 | 2 | CVA07127.1 | 14215 |
| 438 | GCA_001539705.1 | 615 | 615 | Serratia marcescens | 2880STDY5682843 | Proteobacteria | FCGZ01000005.1 | 2 | CUZ96183.1 | 14217 |
| 439 | GCA_001537325.1 | 615 | 615 | Serratia marcescens | 2880STDY5682845 | Proteobacteria | FCHA01000004.1 | 2 | CVA63405.1 | 14219 |
| 440 | GCA_001538805.1 | 615 | 615 | Serratia marcescens | 2880STDY5682847 | Proteobacteria | FCHB01000006.1 | 2 | CVB19728.1 | 14221 |
| 441 | GCA_001537025.1 | 615 | 615 | Serratia marcescens | 2880STDY5682846 | Proteobacteria | FCHC01000005.1 | 2 | CVB15259.1 | 14223 |
| 442 | GCA_001538445.1 | 615 | 615 | Serratia marcescens | 2880STDY5682848 | Proteobacteria | FCHD01000004.1 | 2 | CVB18641.1 | 14225 |
| 443 | GCA_001540265.1 | 615 | 615 | Serratia marcescens | 2880STDY5682849 | Proteobacteria | FCHE01000016.1 | 2 | CVC33418.1 | 14227 |
| 444 | GCA_001539365.1 | 615 | 615 | Serratia marcescens | 2880STDY5682850 | Proteobacteria | FCHF01000016.1 | 2 | CVC43508.1 | 14229 |
| 445 | GCA_001537605.1 | 615 | 615 | Serratia marcescens | 2880STDY5682851 | Proteobacteria | FCHG01000004.1 | 2 | CVB44191.1 | 14231 |
| 446 | GCA_001538725.1 | 615 | 615 | Serratia marcescens | 2880STDY5682852 | Proteobacteria | FCHH01000006.1 | 2 | CVC13530.1 | 14233 |
| 447 | GCA_001536765.1 | 615 | 615 | Serratia marcescens | 2880STDY5682859 | Proteobacteria | FCHI01000001.1 | 2 | CVB24153.1 | 14235 |
| 448 | GCA_001540425.1 | 615 | 615 | Serratia marcescens | 2880STDY5682855 | Proteobacteria | FCHJ01000002.1 | 2 | CVB63296.1 | 14237 |
| 449 | GCA_001539265.1 | 615 | 615 | Serratia marcescens | 2880STDY5682854 | Proteobacteria | FCHK01000007.1 | 2 | CVB59022.1 | 14239 |
| 450 | GCA_001536505.1 | 615 | 615 | Serratia marcescens | 2880STDY5682857 | Proteobacteria | FCHL01000009.1 | 2 | CVB73355.1 | 14241 |
| 451 | GCA_001539765.1 | 615 | 615 | Serratia marcescens | 2880STDY5682858 | Proteobacteria | FCHM01000001.1 | 2 | CVA22576.1 | 14243 |
| 452 | GCA_001536445.1 | 615 | 615 | Serratia marcescens | 2880STDY5682856 | Proteobacteria | FCHN01000007.1 | 2 | CVC54922.1 | 14245 |
| 453 | GCA_001536345.1 | 615 | 615 | Serratia marcescens | 2880STDY5682861 | Proteobacteria | FCHO01000009.1 | 2 | CVC47630.1 | 14247 |
| 454 | GCA_001539585.1 | 615 | 615 | Serratia marcescens | 2880STDY5682863 | Proteobacteria | FCHP01000005.1 | 2 | CVC16906.1 | 14249 |
| 455 | GCA_001540365.1 | 615 | 615 | Serratia marcescens | 2880STDY5682862 | Proteobacteria | FCHQ01000002.1 | 2 | CVA94601.1 | 14251 |
| 456 | GCA_001536465.1 | 615 | 615 | Serratia marcescens | 2880STDY5682864 | Proteobacteria | FCHR01000005.1 | 2 | CVB98029.1 | 14253 |
| 457 | GCA_001537225.1 | 615 | 615 | Serratia marcescens | 2880STDY5682866 | Proteobacteria | FCHS01000005.1 | 2 | CVB27596.1 | 14255 |
| 458 | GCA_001539885.1 | 615 | 615 | Serratia marcescens | 2880STDY5682865 | Proteobacteria | FCHT01000005.1 | 2 | CVC12754.1 | 14257 |
| 459 | GCA_001539845.1 | 615 | 615 | Serratia marcescens | 2880STDY5682867 | Proteobacteria | FCHU01000005.1 | 2 | CVB94192.1 | 14259 |
| 460 | GCA_001538425.1 | 615 | 615 | Serratia marcescens | 2880STDY5682868 | Proteobacteria | FCHV01000005.1 | 2 | CVC34459.1 | 14261 |
| 461 | GCA_001537485.1 | 615 | 615 | Serratia marcescens | 2880STDY5682869 | Proteobacteria | FCHW01000006.1 | 2 | CVC37208.1 | 14263 |
| 462 | GCA_001540525.1 | 615 | 615 | Serratia marcescens | 2880STDY5682870 | Proteobacteria | FCHX01000005.1 | 2 | CVC09911.1 | 14265 |
| 463 | GCA_001537245.1 | 615 | 615 | Serratia marcescens | 2880STDY5682871 | Proteobacteria | FCHY01000004.1 | 2 | CVB52010.1 | 14267 |
| 464 | GCA_001537345.1 | 615 | 615 | Serratia marcescens | 2880STDY5682872 | Proteobacteria | FCHZ01000007.1 | 2 | CVB83712.1 | 14269 |
| 465 | GCA_001536985.1 | 615 | 615 | Serratia marcescens | 2880STDY5682874 | Proteobacteria | FCIA01000006.1 | 2 | CVB86127.1 | 14271 |
| 466 | GCA_001539925.1 | 615 | 615 | Serratia marcescens | 2880STDY5682875 | Proteobacteria | FCIB01000006.1 | 2 | CVC63966.1 | 14273 |
| 467 | GCA_001539425.1 | 615 | 615 | Serratia marcescens | 2880STDY5682877 | Proteobacteria | FCIC01000002.1 | 2 | CVB86015.1 | 14275 |
| 468 | GCA_001537465.1 | 615 | 615 | Serratia marcescens | 2880STDY5682878 | Proteobacteria | FCID01000006.1 | 2 | CVC85484.1 | 14277 |
| 469 | GCA_001540385.1 | 615 | 615 | Serratia marcescens | 2880STDY5682880 | Proteobacteria | FCIE01000007.1 | 2 | CVC33567.1 | 14279 |
| 470 | GCA_001540605.1 | 615 | 615 | Serratia marcescens | 2880STDY5682879 | Proteobacteria | FCIF01000008.1 | 2 | CVC90980.1 | 14281 |
| 471 | GCA_001537665.1 | 615 | 615 | Serratia marcescens | 2880STDY5682881 | Proteobacteria | FCIG01000006.1 | 2 | CVC23364.1 | 14283 |
| 472 | GCA_001539345.1 | 615 | 615 | Serratia marcescens | 2880STDY5682884 | Proteobacteria | FCIH01000009.1 | 2 | CVC01499.1 | 14285 |
| 473 | GCA_001540625.1 | 615 | 615 | Serratia marcescens | 2880STDY5682885 | Proteobacteria | FCII01000005.1 | 2 | CVC17928.1 | 14287 |
| 474 | GCA_001536685.1 | 615 | 615 | Serratia marcescens | 2880STDY5682882 | Proteobacteria | FCIJ01000008.1 | 2 | CVC49211.1 | 14289 |
| 475 | GCA_001538825.1 | 615 | 615 | Serratia marcescens | 2880STDY5682883 | Proteobacteria | FCIK01000010.1 | 2 | CVD13430.1 | 14291 |
| 476 | GCA_001538365.1 | 615 | 615 | Serratia marcescens | 2880STDY5682887 | Proteobacteria | FCIL01000005.1 | 2 | CVC16315.1 | 14293 |
| 477 | GCA_001539745.1 | 615 | 615 | Serratia marcescens | 2880STDY5682889 | Proteobacteria | FCIM01000006.1 | 2 | CVC38417.1 | 14295 |
| 478 | GCA_001538345.1 | 615 | 615 | Serratia marcescens | 2880STDY5682888 | Proteobacteria | FCIN01000006.1 | 2 | CVC81451.1 | 14297 |
| 479 | GCA_001540765.1 | 615 | 615 | Serratia marcescens | 2880STDY5682892 | Proteobacteria | FCIO01000006.1 | 2 | CVC81532.1 | 14299 |
| 480 | GCA_001540325.1 | 615 | 615 | Serratia marcescens | 2880STDY5682891 | Proteobacteria | FCIP01000007.1 | 2 | CVC86644.1 | 14301 |
| 481 | GCA_001540305.1 | 615 | 615 | Serratia marcescens | 2880STDY5682890 | Proteobacteria | FCIQ01000005.1 | 2 | CVC98809.1 | 14303 |
| 482 | GCA_001539565.1 | 615 | 615 | Serratia marcescens | 2880STDY5682893 | Proteobacteria | FCIR01000006.1 | 2 | CVC91105.1 | 14305 |
| 483 | GCA_001540105.1 | 615 | 615 | Serratia marcescens | 2880STDY5682902 | Proteobacteria | FCIS01000005.1 | 2 | CVC94637.1 | 14307 |
| 484 | GCA_001539865.1 | 615 | 615 | Serratia marcescens | 2880STDY5682903 | Proteobacteria | FCIT01000007.1 | 2 | CVC52261.1 | 14309 |
| 485 | GCA_001539085.1 | 615 | 615 | Serratia marcescens | 2880STDY5682904 | Proteobacteria | FCIU01000005.1 | 2 | CVC98188.1 | 14311 |
| 486 | GCA_001537065.1 | 615 | 615 | Serratia marcescens | 2880STDY5682905 | Proteobacteria | FCIV01000001.1 | 2 | CVB21709.1 | 14313 |
| 487 | GCA_001536725.1 | 615 | 615 | Serratia marcescens | 2880STDY5682906 | Proteobacteria | FCIW01000004.1 | 2 | CVC48171.1 | 14315 |
| 488 | GCA_001538645.1 | 615 | 615 | Serratia marcescens | 2880STDY5682907 | Proteobacteria | FCIX01000005.1 | 2 | CVC71605.1 | 14317 |
| 489 | GCA_001539145.1 | 615 | 615 | Serratia marcescens | 2880STDY5682908 | Proteobacteria | FCIY01000004.1 | 2 | CVC84156.1 | 14319 |
| 490 | GCA_001539385.1 | 615 | 615 | Serratia marcescens | 2880STDY5682913 | Proteobacteria | FCIZ01000002.1 | 2 | CVC59811.1 | 14321 |
| 491 | GCA_001537385.1 | 1805636 | 1805636 | Serratia sp. 2880STDY5682894 | 2880STDY5682894 | Proteobacteria | FCJA01000011.1 | 2 | CVE09029.1 | 14323 |
| 492 | GCA_001537125.1 | 615 | 615 | Serratia marcescens | 2880STDY5682916 | Proteobacteria | FCJB01000006.1 | 2 | CVD69842.1 | 14325 |
| 493 | GCA_001540245.1 | 615 | 615 | Serratia marcescens | 2880STDY5682918 | Proteobacteria | FCJC01000003.1 | 2 | CVD51978.1 | 14327 |
| 494 | GCA_001540285.1 | 615 | 615 | Serratia marcescens | 2880STDY5682917 | Proteobacteria | FCJD01000005.1 | 2 | CVD94616.1 | 14329 |
| 495 | GCA_001540485.1 | 615 | 615 | Serratia marcescens | 2880STDY5682919 | Proteobacteria | FCJE01000006.1 | 2 | CVD41075.1 | 14331 |
| 496 | GCA_001539245.1 | 615 | 615 | Serratia marcescens | 2880STDY5682921 | Proteobacteria | FCJF01000005.1 | 2 | CVD28911.1 | 14333 |
| 497 | GCA_001537185.1 | 615 | 615 | Serratia marcescens | 2880STDY5682924 | Proteobacteria | FCJG01000001.1 | 2 | CVC37101.1 | 14335 |
| 498 | GCA_001537625.1 | 615 | 615 | Serratia marcescens | 2880STDY5682926 | Proteobacteria | FCJH01000006.1 | 2 | CVE33560.1 | 14337 |
| 499 | GCA_001536385.1 | 615 | 615 | Serratia marcescens | 2880STDY5682927 | Proteobacteria | FCJI01000001.1 | 2 | CVC40389.1 | 14339 |
| 500 | GCA_001536405.1 | 615 | 615 | Serratia marcescens | 2880STDY5682928 | Proteobacteria | FCJJ01000009.1 | 2 | CVE27784.1 | 14341 |
| 501 | GCA_001536845.1 | 615 | 615 | Serratia marcescens | 2880STDY5682929 | Proteobacteria | FCJK01000007.1 | 2 | CVE60658.1 | 14343 |
| 502 | GCA_001537265.1 | 1805637 | 1805637 | Serratia sp. 2880STDY5682895 | 2880STDY5682895 | Proteobacteria | FCJL01000004.1 | 2 | CVE69225.1 | 14345 |
| 503 | GCA_001540145.1 | 615 | 615 | Serratia marcescens | 2880STDY5682932 | Proteobacteria | FCJM01000004.1 | 2 | CVE06185.1 | 14347 |
| 504 | GCA_001538885.1 | 615 | 615 | Serratia marcescens | 2880STDY5682931 | Proteobacteria | FCJN01000006.1 | 2 | CVE70644.1 | 14349 |
| 505 | GCA_001538945.1 | 615 | 615 | Serratia marcescens | 2880STDY5682933 | Proteobacteria | FCJO01000002.1 | 2 | CVD68108.1 | 14351 |
| 506 | GCA_001539285.1 | 615 | 615 | Serratia marcescens | 2880STDY5682936 | Proteobacteria | FCJP01000008.1 | 2 | CVE19652.1 | 14353 |
| 507 | GCA_001538965.1 | 615 | 615 | Serratia marcescens | 2880STDY5682938 | Proteobacteria | FCJQ01000005.1 | 2 | CVE94858.1 | 14355 |
| 508 | GCA_001538745.1 | 615 | 615 | Serratia marcescens | 2880STDY5682934 | Proteobacteria | FCJR01000005.1 | 2 | CVE73969.1 | 14357 |
| 509 | GCA_001539405.1 | 615 | 615 | Serratia marcescens | 2880STDY5682937 | Proteobacteria | FCJS01000004.1 | 2 | CVE69307.1 | 14359 |
| 510 | GCA_001540745.1 | 615 | 615 | Serratia marcescens | 2880STDY5682896 | Proteobacteria | FCJU01000008.1 | 2 | CVD84033.1 | 14361 |
| 511 | GCA_001540025.1 | 615 | 615 | Serratia marcescens | 2880STDY5682940 | Proteobacteria | FCJV01000010.1 | 2 | CVE29057.1 | 14363 |
| 512 | GCA_001539625.1 | 615 | 615 | Serratia marcescens | 2880STDY5682943 | Proteobacteria | FCJW01000006.1 | 2 | CVD89429.1 | 14365 |
| 513 | GCA_001536965.1 | 615 | 615 | Serratia marcescens | 2880STDY5682942 | Proteobacteria | FCJX01000004.1 | 2 | CVE62947.1 | 14367 |
| 514 | GCA_001538505.1 | 615 | 615 | Serratia marcescens | 2880STDY5682945 | Proteobacteria | FCJY01000006.1 | 2 | CVE95724.1 | 14369 |
| 515 | GCA_001538005.1 | 615 | 615 | Serratia marcescens | 2880STDY5682947 | Proteobacteria | FCJZ01000006.1 | 2 | CVF11414.1 | 14371 |
| 516 | GCA_001537005.1 | 615 | 615 | Serratia marcescens | 2880STDY5682951 | Proteobacteria | FCKA01000009.1 | 2 | CVF28618.1 | 14373 |
| 517 | GCA_001536895.1 | 615 | 615 | Serratia marcescens | 2880STDY5682950 | Proteobacteria | FCKB01000001.1 | 2 | CVC97408.1 | 14375 |
| 518 | GCA_001537365.1 | 615 | 615 | Serratia marcescens | 2880STDY5682897 | Proteobacteria | FCKC01000002.1 | 2 | CVD52669.1 | 14377 |
| 519 | GCA_001536525.1 | 615 | 615 | Serratia marcescens | 2880STDY5682952 | Proteobacteria | FCKD01000005.1 | 2 | CVE22718.1 | 14379 |
| 520 | GCA_001539545.1 | 615 | 615 | Serratia marcescens | 2880STDY5682953 | Proteobacteria | FCKE01000005.1 | 2 | CVE97488.1 | 14381 |
| 521 | GCA_001539665.1 | 615 | 615 | Serratia marcescens | 2880STDY5682954 | Proteobacteria | FCKF01000005.1 | 2 | CVF28196.1 | 14383 |
| 522 | GCA_001536325.1 | 615 | 615 | Serratia marcescens | 2880STDY5682955 | Proteobacteria | FCKG01000004.1 | 2 | CVE60557.1 | 14385 |
| 523 | GCA_001539325.1 | 615 | 615 | Serratia marcescens | 2880STDY5682956 | Proteobacteria | FCKH01000006.1 | 2 | CVE66029.1 | 14387 |
| 524 | GCA_001540825.1 | 615 | 615 | Serratia marcescens | 2880STDY5682957 | Proteobacteria | FCKI01000006.1 | 2 | CVF01432.1 | 14389 |
| 525 | GCA_001540005.1 | 615 | 615 | Serratia marcescens | 2880STDY5682958 | Proteobacteria | FCKJ01000007.1 | 2 | CVE93279.1 | 14391 |
| 526 | GCA_001536625.1 | 615 | 615 | Serratia marcescens | 2880STDY5682959 | Proteobacteria | FCKK01000007.1 | 2 | CVF50179.1 | 14393 |
| 527 | GCA_001539905.1 | 615 | 615 | Serratia marcescens | 2880STDY5682961 | Proteobacteria | FCKL01000003.1 | 2 | CVF09863.1 | 14395 |
| 528 | GCA_001536665.1 | 615 | 615 | Serratia marcescens | 2880STDY5682960 | Proteobacteria | FCKM01000009.1 | 2 | CVE63433.1 | 14397 |
| 529 | GCA_001539205.1 | 615 | 615 | Serratia marcescens | 2880STDY5682962 | Proteobacteria | FCKN01000002.1 | 2 | CVE05426.1 | 14399 |
| 530 | GCA_001540685.1 | 615 | 615 | Serratia marcescens | 2880STDY5682899 | Proteobacteria | FCKO01000003.1 | 2 | CVE67637.1 | 14401 |
| 531 | GCA_001539945.1 | 615 | 615 | Serratia marcescens | 2880STDY5682963 | Proteobacteria | FCKP01000005.1 | 2 | CVE73588.1 | 14403 |
| 532 | GCA_001536705.1 | 615 | 615 | Serratia marcescens | 2880STDY5682964 | Proteobacteria | FCKQ01000006.1 | 2 | CVF13093.1 | 14405 |
| 533 | GCA_001539445.1 | 615 | 615 | Serratia marcescens | 2880STDY5682965 | Proteobacteria | FCKR01000003.1 | 2 | CVE92971.1 | 14407 |
| 534 | GCA_001538685.1 | 615 | 615 | Serratia marcescens | 2880STDY5682967 | Proteobacteria | FCKS01000007.1 | 2 | CVF15693.1 | 14409 |
| 535 | GCA_001536945.1 | 615 | 615 | Serratia marcescens | 2880STDY5682966 | Proteobacteria | FCKT01000001.1 | 2 | CVE70328.1 | 14411 |
| 536 | GCA_001538405.1 | 615 | 615 | Serratia marcescens | 2880STDY5682968 | Proteobacteria | FCKU01000005.1 | 2 | CVF77852.1 | 14413 |
| 537 | GCA_001538115.1 | 615 | 615 | Serratia marcescens | 2880STDY5682971 | Proteobacteria | FCKV01000009.1 | 2 | CVF08013.1 | 14415 |
| 538 | GCA_001540545.1 | 615 | 615 | Serratia marcescens | 2880STDY5682969 | Proteobacteria | FCKW01000004.1 | 2 | CVF84175.1 | 14417 |
| 539 | GCA_001540345.1 | 615 | 615 | Serratia marcescens | 2880STDY5682970 | Proteobacteria | FCKX01000005.1 | 2 | CVF82181.1 | 14419 |
| 540 | GCA_001540705.1 | 615 | 615 | Serratia marcescens | 2880STDY5682973 | Proteobacteria | FCKZ01000006.1 | 2 | CVG26740.1 | 14421 |
| 541 | GCA_001537585.1 | 615 | 615 | Serratia marcescens | 2880STDY5682974 | Proteobacteria | FCLA01000006.1 | 2 | CVG63931.1 | 14423 |
| 542 | GCA_001540445.1 | 615 | 615 | Serratia marcescens | 2880STDY5682976 | Proteobacteria | FCLB01000006.1 | 2 | CVG49466.1 | 14425 |
| 543 | GCA_001536645.1 | 615 | 615 | Serratia marcescens | 2880STDY5682977 | Proteobacteria | FCLC01000007.1 | 2 | CVF96122.1 | 14427 |
| 544 | GCA_001538585.1 | 615 | 615 | Serratia marcescens | 2880STDY5682979 | Proteobacteria | FCLD01000006.1 | 2 | CVG60878.1 | 14429 |
| 545 | GCA_001539825.1 | 615 | 615 | Serratia marcescens | 2880STDY5682978 | Proteobacteria | FCLE01000011.1 | 2 | CVG18609.1 | 14431 |
| 546 | GCA_001536365.1 | 615 | 615 | Serratia marcescens | 2880STDY5682980 | Proteobacteria | FCLF01000004.1 | 2 | CVG60220.1 | 14433 |
| 547 | GCA_001540505.1 | 615 | 615 | Serratia marcescens | 2880STDY5682982 | Proteobacteria | FCLG01000005.1 | 2 | CVG00307.1 | 14435 |
| 548 | GCA_001540045.1 | 615 | 615 | Serratia marcescens | 2880STDY5682981 | Proteobacteria | FCLH01000005.1 | 2 | CVG18150.1 | 14437 |
| 549 | GCA_001539685.1 | 615 | 615 | Serratia marcescens | 2880STDY5682983 | Proteobacteria | FCLI01000006.1 | 2 | CVG66356.1 | 14439 |
| 550 | GCA_001539605.1 | 615 | 615 | Serratia marcescens | 2880STDY5682984 | Proteobacteria | FCLJ01000002.1 | 2 | CVF82688.1 | 14441 |
| 551 | GCA_001564475.1 | 615 | 615 | Serratia marcescens | 2880STDY5682985 | Proteobacteria | FCLK01000006.1 | 2 | CVG27221.1 | 14443 |
| 552 | GCA_001537705.1 | 615 | 615 | Serratia marcescens | 2880STDY5682986 | Proteobacteria | FCLL01000011.1 | 2 | CVH08342.1 | 14445 |
| 553 | GCA_001540785.1 | 615 | 615 | Serratia marcescens | 2880STDY5682987 | Proteobacteria | FCLM01000006.1 | 2 | CVG82373.1 | 14447 |
| 554 | GCA_001540065.1 | 615 | 615 | Serratia marcescens | 2880STDY5682990 | Proteobacteria | FCLN01000007.1 | 2 | CVG89997.1 | 14449 |
| 555 | GCA_001540585.1 | 615 | 615 | Serratia marcescens | 2880STDY5682991 | Proteobacteria | FCLO01000004.1 | 2 | CVG75154.1 | 14451 |
| 556 | GCA_001536745.1 | 615 | 615 | Serratia marcescens | 2880STDY5682992 | Proteobacteria | FCLP01000006.1 | 2 | CVG91933.1 | 14453 |
| 557 | GCA_001538865.1 | 615 | 615 | Serratia marcescens | 2880STDY5682993 | Proteobacteria | FCLQ01000006.1 | 2 | CVG61761.1 | 14455 |
| 558 | GCA_001536545.1 | 615 | 615 | Serratia marcescens | 2880STDY5682994 | Proteobacteria | FCLR01000006.1 | 2 | CVG55708.1 | 14457 |
| 559 | GCA_001537925.1 | 615 | 615 | Serratia marcescens | 2880STDY5682995 | Proteobacteria | FCLS01000005.1 | 2 | CVG99558.1 | 14459 |
| 560 | GCA_001538925.1 | 615 | 615 | Serratia marcescens | 2880STDY5682901 | Proteobacteria | FCLT01000009.1 | 2 | CVG85830.1 | 14461 |
| 561 | GCA_001538845.1 | 615 | 615 | Serratia marcescens | 2880STDY5682996 | Proteobacteria | FCLU01000006.1 | 2 | CVH13287.1 | 14463 |
| 562 | GCA_001536425.1 | 615 | 615 | Serratia marcescens | 2880STDY5682998 | Proteobacteria | FCLV01000007.1 | 2 | CVH22095.1 | 14465 |
| 563 | GCA_001540165.1 | 615 | 615 | Serratia marcescens | 2880STDY5682997 | Proteobacteria | FCLW01000007.1 | 2 | CVG79620.1 | 14467 |
| 564 | GCA_001540565.1 | 615 | 615 | Serratia marcescens | 2880STDY5683002 | Proteobacteria | FCLX01000006.1 | 2 | CVH31735.1 | 14469 |
| 565 | GCA_001537085.1 | 615 | 615 | Serratia marcescens | 2880STDY5683001 | Proteobacteria | FCLY01000002.1 | 2 | CVG61510.1 | 14471 |
| 566 | GCA_001539005.1 | 615 | 615 | Serratia marcescens | 2880STDY5682873 | Proteobacteria | FCLZ01000001.1 | 2 | CVF47731.1 | 14473 |
| 567 | GCA_001538565.1 | 615 | 615 | Serratia marcescens | 2880STDY5683004 | Proteobacteria | FCMA01000007.1 | 2 | CVH23270.1 | 14475 |
| 568 | GCA_001537745.1 | 615 | 615 | Serratia marcescens | 2880STDY5683003 | Proteobacteria | FCMB01000010.1 | 2 | CVG86995.1 | 14477 |
| 569 | GCA_001538385.1 | 615 | 615 | Serratia marcescens | 2880STDY5682835 | Proteobacteria | FCMC01000007.1 | 2 | CVG92563.1 | 14479 |
| 570 | GCA_001537685.1 | 615 | 615 | Serratia marcescens | 2880STDY5682853 | Proteobacteria | FCMD01000007.1 | 2 | CVH34087.1 | 14481 |
| 571 | GCA_001537905.1 | 615 | 615 | Serratia marcescens | 2880STDY5682876 | Proteobacteria | FCME01000005.1 | 2 | CVG73776.1 | 14483 |
| 572 | GCA_001537505.1 | 615 | 615 | Serratia marcescens | 2880STDY5682898 | Proteobacteria | FCMF01000005.1 | 2 | CVH31066.1 | 14485 |
| 573 | GCA_001538275.1 | 615 | 615 | Serratia marcescens | 2880STDY5682912 | Proteobacteria | FCMG01000007.1 | 2 | CVH30715.1 | 14487 |
| 574 | GCA_001537045.1 | 615 | 615 | Serratia marcescens | 2880STDY5682914 | Proteobacteria | FCMH01000006.1 | 2 | CVG97897.1 | 14489 |
| 575 | GCA_001540465.1 | 615 | 615 | Serratia marcescens | 2880STDY5682915 | Proteobacteria | FCMI01000011.1 | 2 | CVH42932.1 | 14491 |
| 576 | GCA_001540085.1 | 615 | 615 | Serratia marcescens | 2880STDY5682925 | Proteobacteria | FCMJ01000003.1 | 2 | CVH11685.1 | 14493 |
| 577 | GCA_001536585.1 | 615 | 615 | Serratia marcescens | 2880STDY5682922 | Proteobacteria | FCMK01000007.1 | 2 | CVH39732.1 | 14495 |
| 578 | GCA_001537305.1 | 615 | 615 | Serratia marcescens | 2880STDY5682930 | Proteobacteria | FCML01000005.1 | 2 | CVH33868.1 | 14497 |
| 579 | GCA_001538325.1 | 615 | 615 | Serratia marcescens | 2880STDY5682939 | Proteobacteria | FCMM01000005.1 | 2 | CVG91272.1 | 14499 |
| 580 | GCA_001537165.1 | 615 | 615 | Serratia marcescens | 2880STDY5682944 | Proteobacteria | FCMN01000005.1 | 2 | CVH37766.1 | 14501 |
| 581 | GCA_001540405.1 | 615 | 615 | Serratia marcescens | 2880STDY5682948 | Proteobacteria | FCMO01000007.1 | 2 | CVH21028.1 | 14503 |
| 582 | GCA_001537865.1 | 615 | 615 | Serratia marcescens | 2880STDY5682949 | Proteobacteria | FCMP01000001.1 | 2 | CVG01710.1 | 14505 |
| 583 | GCA_001538665.1 | 615 | 615 | Serratia marcescens | 2880STDY5682975 | Proteobacteria | FCMQ01000009.1 | 2 | CVH35272.1 | 14507 |
| 584 | GCA_001536565.1 | 615 | 615 | Serratia marcescens | 2880STDY5682999 | Proteobacteria | FCMS01000001.1 | 2 | CVH07248.1 | 14509 |
| 585 | GCA_001539465.1 | 615 | 615 | Serratia marcescens | 2880STDY5683000 | Proteobacteria | FCMT01000008.1 | 2 | CVH30929.1 | 14511 |
| 586 | GCA_001537805.1 | 615 | 615 | Serratia marcescens | 2880STDY5683016 | Proteobacteria | FCMU01000006.1 | 2 | CVH50698.1 | 14513 |
| 587 | GCA_001539525.1 | 615 | 615 | Serratia marcescens | 2880STDY5683027 | Proteobacteria | FCMV01000005.1 | 2 | CVH40520.1 | 14515 |
| 588 | GCA_001539105.1 | 615 | 615 | Serratia marcescens | 2880STDY5683015 | Proteobacteria | FCMW01000009.1 | 2 | CVH64627.1 | 14517 |
| 589 | GCA_001537785.1 | 615 | 615 | Serratia marcescens | 2880STDY5683006 | Proteobacteria | FCMX01000005.1 | 2 | CVH70545.1 | 14519 |
| 590 | GCA_001540645.1 | 615 | 615 | Serratia marcescens | 2880STDY5683019 | Proteobacteria | FCMY01000005.1 | 2 | CVH72214.1 | 14521 |
| 591 | GCA_001538545.1 | 615 | 615 | Serratia marcescens | 2880STDY5683017 | Proteobacteria | FCMZ01000004.1 | 2 | CVH65591.1 | 14523 |
| 592 | GCA_001544575.1 | 1353886 | 1353886 | Burkholderia cordobensis | | Proteobacteria | FCNY01000004.1 | 2 | CVJ13885.1 | 14525 |
| 593 | GCA_001544455.1 | 196367 | 196367 | Paraburkholderia sordidicola | | Proteobacteria | FCOC01000002.1 | 3 | CVJ11200.1 | 14527 |
| 594 | GCA_001544535.1 | 326476 | 326476 | Paraburkholderia choica | | Proteobacteria | FCON01000032.1 | 2 | CVJ34937.1 | 14530 |
| 595 | GCA_900022065.1 | 1639 | 1639 | Listeria monocytogenes | 2842STDY5753889 | Firmicutes | FFEW01000009.1 | 2 | CWV39688.1 | 14532 |
| 596 | GCA_900034805.1 | 1639 | 1639 | Listeria monocytogenes | 2842STDY5753890 | Firmicutes | FFGS01000008.1 | 2 | CWW37500.1 | 14534 |
| 597 | GCA_900051225.1 | 1313 | 1313 | Streptococcus pneumoniae | 2245STDY5982743 | Firmicutes | FHVI01000002.1 | 3 | CYO51328.1 | 14536 |
| 598 | GCA_900059415.1 | 1307 | 1307 | Streptococcus suis | LSS12 | Firmicutes | FIFP01000001.1 | 2 | CYT98259.1 | 14539 |
| 599 | GCA_900049685.1 | 1307 | 1307 | Streptococcus suis | FX275 | Firmicutes | FISX01000005.1 | 3 | CZB10988.1 | 14541 |
| 600 | GCA_900058075.1 | 1307 | 1307 | Streptococcus suis | FX483_3 | Firmicutes | FITF01000040.1 | 3 | CZB35771.1 | 14544 |
| 601 | GCA_900050515.1 | 1313 | 1313 | Streptococcus pneumoniae | 72D37909 | Firmicutes | FIWN01000018.1 | 2 | CZD24115.1 | 14547 |
| 602 | GCA_900052575.1 | 1313 | 1313 | Streptococcus pneumoniae | D30716 | Firmicutes | FIXA01000003.1 | 2 | CZC77630.1 | 14549 |
| 603 | GCA_900049745.1 | 1313 | 1313 | Streptococcus pneumoniae | A32279 | Firmicutes | FIXS01000003.1 | 2 | CZD72536.1 | 14551 |
| 604 | GCA_900051625.1 | 1313 | 1313 | Streptococcus pneumoniae | C14560X | Firmicutes | FIXT01000039.1 | 2 | CZE23674.1 | 14553 |
| 605 | GCA_000026585.1 | 553482 | 1336 | Streptococcus equi subsp. equi | 4047 | Firmicutes | FM204883.1 | 2 | CAW93164.1 | 14555 |
| | | | | 4047 | | | | | | |
| 606 | GCA_000209955.1 | 657317 | 39491 | [Eubacterium] rectale M104/1 | M104/1 | Firmicutes | FP929043.1 | 2 | CBK94114.1 | 14557 |
| 607 | GCA_000236925.1 | 985002 | 985002 | Staphylococcus argenteus | MSHR1132 | Firmicutes | FR821777.2 | 3 | CCE59808.1 | 14559 |
| 608 | GCA_000435855.1 | 1262967 | 1262967 | Ruminococcus sp. CAG:9 | | Firmicutes | FR896087.1 | 3 | CDD80086.1 | 14562 |
| 609 | GCA_000156515.1 | 553973 | 89153 | [Clostridium] hylemonae DSM 15053 | DSM 15053 | Firmicutes | GG657761.1 | 2 | EEG72544.1 | 14565 |
| 610 | GCA_000160115.1 | 626523 | 177972 | Shuttleworthia satelles DSM 14600 | DSM 14600 | Firmicutes | GG665867.1 | 4 | EEP27313.1 | 14567 |
| 611 | GCA_000159095.1 | 525255 | 33036 | Anaerococcus tetradius ATCC 35098 | ATCC 35098 | Firmicutes | GG666297.1 | 2 | EEI83075.1 | 14571 |
| 612 | GCA_000157535.1 | 565657 | 1352 | Enterococcus faecium 1,231,502 | 1,231,502 | Firmicutes | GG688488.1 | 3 | EEV44995.1 | 14573 |
| 613 | GCA_000159955.1 | 621372 | 652715 | Paenibacillus sp. oral taxon 786 str. D14 | D14 | Firmicutes | GG695991.1 | 2 | EES71591.1 | 14576 |
| 614 | GCA_000162075.1 | 575587 | 40215 | Acinetobacter junii SH205 | SH205 | Proteobacteria | GG705011.1 | 3 | EEY94494.1 | 14578 |
| 615 | GCA_000148025.1 | 749520 | 1352 | Enterococcus faecium TX0082 | TX0082 | Firmicutes | GL455872.1 | 3 | EFS10290.1 | 14581 |
| 616 | GCA_000195045.1 | 888818 | 1305 | Streptococcus sanguinis SK408 | SK408 | Firmicutes | GL878559.1 | 3 | EGF19709.1 | 14584 |
| 617 | GCA_000213555.1 | 742766 | 156974 | Dysgonomonas gadei ATCC BAA-286 | ATCC BAA-286 | Bacteroidetes | GL891989.1 | 3 | EGK00063.1 | 14587 |
| 618 | GCA_000328705.1 | 1179773 | 103731 | Saccharothrix espanaensis DSM 44229 | type strain: | Actinobacteria | HE804045.1 | 2 | CCH29383.1 | 14590 |
| | | | | | DSM 44229 | | | | | |
| 619 | GCA_000313065.1 | 1208659 | 518 | Bordetella bronchiseptica Bbr77 | Bbr77 | Proteobacteria | HE983628.1 | 2 | CCN04569.1 | 14592 |
| 620 | GCA_000513215.1 | 273526 | 615 | Serratia marcescens subsp. marcescens Db11 | Db11 | Proteobacteria | HG326223.1 | 2 | CDG13494.1 | 14594 |
| 621 | GCA_000736695.1 | 1398204 | 40576 | Xenorhabdus bovienii str. kraussei Becker | kraussei Becker | Proteobacteria | HG428180.1 | 2 | CDH26046.1 | 14596 |
| | | | | Underwood | Underwood | | | | | |
| 622 | GCA_000632355.1 | 1442605 | 1352 | Enterococcus faecium VSE1036 | VSE1036 | Firmicutes | JAAJ01000002.1 | 3 | EZP94706.1 | 14598 |
| 623 | GCA_000632295.1 | 1442604 | 1352 | Enterococcus faecium VRE0576 | VRE0576 | Firmicutes | JAAK01000036.1 | 3 | EZP99085.1 | 14601 |
| 624 | GCA_000632315.1 | 1442606 | 1352 | Enterococcus faecium VRE1044 | VRE1044 | Firmicutes | JAAL01000002.1 | 3 | EZP92278.1 | 14604 |
| 625 | GCA_000523375.1 | 1449358 | 670 | Vibrio parahaemolyticus M0605 | M0605 | Proteobacteria | JALL01000080.1 | 2 | ETZ11576.1 | 14607 |
| 626 | GCA_000633575.2 | 1450518 | 440518 | Sphingobium lucknowense F2 | F2 | Proteobacteria | JANF02000091.1 | 2 | KER34865.1 | 14609 |
| 627 | GCA_000564995.1 | 1190620 | 1190620 | Atopobium sp. ICM42b | ICM42b | Actinobacteria | JDFH01000020.1 | 2 | EWC94460.1 | 14611 |
| 628 | GCA_001589275.1 | 56 | 56 | Sorangium cellulosum | So0003-19-2 | Proteobacteria | JELZ01001521.1 | 2 | KYF75625.1 | 14613 |
| 629 | GCA_001589185.1 | 56 | 56 | Sorangium cellulosum | So0011-07 | Proteobacteria | JEMB01003116.1 | 2 | KYF75483.1 | 14615 |
| 630 | GCA_000581895.1 | 1310692 | 470 | Acinetobacter baumannii 532279 | 532279 | Proteobacteria | JEYH01000003.1 | 6 | EXE88617.1 | 14617 |
| 631 | GCA_000695685.1 | 212667 | 212667 | Vibrio fortis | Dalian14 | Proteobacteria | JFFR01000027.1 | 2 | KDN26971.1 | 14623 |
| 632 | GCA_000705595.1 | 1408224 | 194963 | Sinorhizobium americanum CCGM7 | CCGM7 | Proteobacteria | JFG001000039.1 | 3 | KEC78309.1 | 14625 |
| 633 | GCA_000689815.1 | 1331227 | 518 | Bordetella bronchiseptica MBORD675 | MBORD675 | Proteobacteria | JGX001000020.1 | 2 | KDC94426.1 | 14628 |
| 634 | GCA_000227315.1 | 997350 | 33030 | Peptoniphilus indolicus ATCC 29427 | ATCC 29427 | Firmicutes | JH165061.1 | 2 | EGY80387.1 | 14630 |
| 635 | GCA_000234335.1 | 543736 | 37919 | Rhodococcus opacus PD630 | PD630 | Actinobacteria | JH377359.1 | 2 | EHI41035.1 | 14632 |
| 636 | GCA_000235505.1 | 742737 | 154046 | Hungatella hathewayi WAL-18680 | WAL-18680 | Firmicutes | JH379027.1 | 2 | EHI61291.1 | 14634 |
| 637 | GCA_000243215.1 | 742817 | 626933 | Odoribacter laneus YIT 12061 | YIT 12061 | Bacteroidetes | JH594596.1 | 3 | EHP50820.1 | 14636 |
| 638 | GCA_000245755.1 | 883114 | 40091 | Helcococcus kunzii ATCC 51366 | ATCC 51366 | Firmicutes | JH601089.1 | 2 | EHR31702.1 | 14639 |
| 639 | GCA_000273035.1 | 997875 | 357276 | Bacteroides dorei CL02T00C15 | CL02T00C15 | Bacteroidetes | JH724115.1 | 4 | EIY18768.1 | 14641 |
| 640 | GCA_000273055.1 | 997876 | 357276 | Bacteroides dorei CL02T12C06 | CL02T12C06 | Bacteroidetes | JH724132.1 | 4 | EIY40460.1 | 14645 |
| 641 | GCA_000273765.1 | 997880 | 817 | Bacteroides fragilis CL05T00C42 | CL05T00C42 | Bacteroidetes | JH724188.1 | 2 | EIY98048.1 | 14649 |
| 642 | GCA_000273765.1 | 997880 | 817 | Bacteroides fragilis CL05T00C42 | CL05T00C42 | Bacteroidetes | JH724189.1 | 2 | EIY91470.1 | 14651 |
| 643 | GCA_000273765.1 | 997880 | 817 | Bacteroides fragilis CL05T00C42 | CL05T00C42 | Bacteroidetes | JH724190.1 | 2 | EIY90206.1 | 14653 |
| 644 | GCA_000273135.1 | 997881 | 817 | Bacteroides fragilis CL05T12C13 | CL05T12C13 | Bacteroidetes | JH724193.1 | 2 | EIY98754.1 | 14655 |
| 645 | GCA_000273135.1 | 997881 | 817 | Bacteroides fragilis CL05T12C13 | CL05T12C13 | Bacteroidetes | JH724195.1 | 2 | EIY95127.1 | 14657 |
| 646 | GCA_000273135.1 | 997881 | 817 | Bacteroides fragilis CL05T12C13 | CL05T12C13 | Bacteroidetes | JH724196.1 | 2 | EIY94597.1 | 14659 |
| 647 | GCA_000273135.1 | 997881 | 817 | Bacteroides fragilis CL05T12C13 | CL05T12C13 | Bacteroidetes | JH724198.1 | 2 | EIY90857.1 | 14661 |
| 648 | GCA_000290655.1 | 1053250 | 1396 | Bacillus cereus VDM062 | VDM062 | Firmicutes | JH791805.1 | 2 | EJS10770.1 | 14663 |
| 649 | GCA_000290695.1 | 1053247 | 1396 | Bacillus cereus VDM022 | VDM022 | Firmicutes | JH791837.1 | 2 | EJS00271.1 | 14665 |
| 650 | GCA_000291275.1 | 1053222 | 1396 | Bacillus cereus MSX-D12 | MSX-D12 | Firmicutes | JH792019.1 | 3 | EJR09240.1 | 14667 |
| 651 | GCA_000293705.1 | 1053201 | 1396 | Bacillus cereus HuA2-1 | HuA2-1 | Firmicutes | JH804676.1 | 2 | EJV74397.1 | 14670 |
| 652 | GCA_000293985.2 | 1134784 | 1351 | Enterococcus faecalis 599 | 599 | Firmicutes | JH805574.1 | 3 | EJU93680.1 | 14672 |
| 653 | GCA_000294835.2 | 1134840 | 1352 | Enterococcus faecium S447 | S447 | Firmicutes | JH806853.1 | 3 | EJX46905.1 | 14675 |
| 654 | GCA_000294955.2 | 1134834 | 1352 | Enterococcus faecium R446 | R446 | Firmicutes | JH808093.1 | 3 | EJX62525.1 | 14678 |
| 655 | GCA_000294975.2 | 1134833 | 1352 | Enterococcus faecium P1986 | P1986 | Firmicutes | JH808384.1 | 3 | EJX62294.1 | 14681 |
| 656 | GCA_000294915.2 | 1134836 | 1352 | Enterococcus faecium R496 | R496 | Firmicutes | JH808647.1 | 3 | EJX50428.1 | 14684 |
| 657 | GCA_000295015.1 | 1134831 | 1352 | Enterococcus faecium P1140 | P1140 | Firmicutes | JH809135.1 | 3 | EJX67854.1 | 14687 |
| 658 | GCA_000295075.2 | 1134823 | 1352 | Enterococcus faecium ERV168 | ERV168 | Firmicutes | JH809674.1 | 3 | EJX96647.1 | 14690 |
| 659 | GCA_000295235.2 | 1134822 | 1352 | Enterococcus faecium ERV165 | ERV165 | Firmicutes | JH811082.1 | 3 | EJX94481.1 | 14693 |
| 660 | GCA_000295255.2 | 1134821 | 1352 | Enterococcus faecium ERV161 | ERV161 | Firmicutes | JH811267.1 | 3 | EJX98618.1 | 14696 |
| 661 | GCA_000295355.2 | 1134820 | 1352 | Enterococcus faecium ERV102 | ERV102 | Firmicutes | JH811829.1 | 3 | EJY06338.1 | 14699 |
| 662 | GCA_000295395.2 | 1134816 | 1352 | Enterococcus faecium C621 | C621 | Firmicutes | JH813401.1 | 3 | EJY12738.1 | 14702 |
| 663 | GCA_000295455.2 | 1134818 | 1352 | Enterococcus faecium E422 | E422 | Firmicutes | JH814230.1 | 3 | EJY15300.1 | 14705 |
| 664 | GCA_000298035.1 | 1073385 | 654 | Aeromonas veronii AMC35 | AMC35 | Proteobacteria | JH815589.1 | 3 | EKB22093.1 | 14708 |
| 665 | GCA_000302795.1 | 1154761 | 317 | Pseudomonas syringae pv. avellanae str. | ISPaVe013 | Proteobacteria | JH951825.1 | 2 | EKG43671.1 | 14711 |
| | | | | ISPaVe013 | | | | | | |
| 666 | GCA_000601255.1 | 1444050 | 562 | Escherichia coli 1-176-05_S1_C1 | 1-176-05_S1_C1 | Proteobacteria | JHDH01000118.1 | 2 | EYD87721.1 | 14713 |
| 667 | GCA_000682795.1 | 1413578 | 1280 | Staphylococcus aureus ZTA11/03130-3ST | ZTA11/03130-3ST | Firmicutes | JIFC01000010.1 | 3 | EZR33643.1 | 14715 |
| 668 | GCA_000682815.1 | 1413583 | 1280 | Staphylococcus aureus ZTA09/03739-9HSA | ZTA09/03739-9HSA | Firmicutes | JIFJ01000017.1 | 3 | EZR32908.1 | 14718 |
| 669 | GCA_000682835.1 | 1413575 | 1280 | Staphylococcus aureus ZTA09/03576-9HST | ZTA09/03576-9HST | Firmicutes | JIFL01000015.1 | 3 | EZR32079.1 | 14721 |
| 670 | GCA_000682855.1 | 1422806 | 1280 | Staphylococcus aureus VET1918S | VET1918S | Firmicutes | JIFS01000014.1 | 3 | EZR40124.1 | 14724 |
| 671 | GCA_000682875.1 | 1422751 | 1280 | Staphylococcus aureus VET1915R | VET1915R | Firmicutes | JIFU01000012.1 | 3 | EZR39627.1 | 14727 |
| 672 | GCA_000682895.1 | 1422746 | 1280 | Staphylococcus aureus VET1910R | VET1910R | Firmicutes | JIFZ01000014.1 | 3 | EZR44611.1 | 14730 |
| 673 | GCA_000682915.1 | 1422743 | 1280 | Staphylococcus aureus VET1907R | VET1907R | Firmicutes | JIGC01000014.1 | 3 | EZR45907.1 | 14733 |
| 674 | GCA_000682935.1 | 1422737 | 1280 | Staphylococcus aureus VET1898R | VET1898R | Firmicutes | JIGI01000039.1 | 3 | EZR45668.1 | 14736 |
| 675 | GCA_000682955.1 | 1422735 | 1280 | Staphylococcus aureus VET1896R | VET1896R | Firmicutes | JIGK01000023.1 | 3 | EZR50025.1 | 14739 |
| 676 | GCA_000682975.1 | 1422726 | 1280 | Staphylococcus aureus VET1876R | VET1876R | Firmicutes | JIGT01000015.1 | 3 | EZR55035.1 | 14742 |
| 677 | GCA_000682995.1 | 1422721 | 1280 | Staphylococcus aureus VET1869R | VET1869R | Firmicutes | JIGY01000015.1 | 3 | EZR57945.1 | 14745 |
| 678 | GCA_000683015.1 | 1422713 | 1280 | Staphylococcus aureus VET1858R | VET1858R | Firmicutes | JIHG01000011.1 | 3 | EZR61549.1 | 14748 |
| 679 | GCA_000683035.1 | 1422699 | 1280 | Staphylococcus aureus VET1842R | VET1842R | Firmicutes | JIHU01000011.1 | 3 | EZR61336.1 | 14751 |
| 680 | GCA_000683055.1 | 1422694 | 1280 | Staphylococcus aureus VET1833R | VET1833R | Firmicutes | JIHZ01000024.1 | 3 | EZR64331.1 | 14754 |
| 681 | GCA_000683075.1 | 1422793 | 1280 | Staphylococcus aureus VET1515S | VET1515S | Firmicutes | JIIN01000014.1 | 3 | EZR68241.1 | 14757 |
| 682 | GCA_000683095.1 | 1422788 | 1280 | Staphylococcus aureus VET1422S | VET1422S | Firmicutes | JIIS01000014.1 | 3 | EZR69014.1 | 14760 |
| 683 | GCA_000683115.1 | 1422787 | 1280 | Staphylococcus aureus VET1419S | VET1419S | Firmicutes | JIIT01000011.1 | 3 | EZR71841.1 | 14763 |
| 684 | GCA_000683175.1 | 1422779 | 1280 | Staphylococcus aureus VET1103S | VET1103S | Firmicutes | JIJB01000012.1 | 3 | EZR76991.1 | 14766 |
| 685 | GCA_000683195.1 | 1422762 | 1280 | Staphylococcus aureus VET0798S | VET0798S | Firmicutes | JIJR01000008.1 | 3 | EZR83388.1 | 14769 |
| 686 | GCA_000683215.1 | 1422759 | 1280 | Staphylococcus aureus VET0765S | VET0765S | Firmicutes | JIJU01000014.1 | 3 | EZR85552.1 | 14772 |
| 687 | GCA_000683235.1 | 1422753 | 1280 | Staphylococcus aureus VET0612S | VET0612S | Firmicutes | JIKA01000011.1 | 3 | EZR86482.1 | 14775 |
| 688 | GCA_000683255.1 | 1422690 | 1280 | Staphylococcus aureus VET0489R | VET0489R | Firmicutes | JIKD01000012.1 | 3 | EZR91407.1 | 14778 |
| 689 | GCA_000683275.1 | 1422684 | 1280 | Staphylococcus aureus VET0473R | VET0473R | Firmicutes | JIKJ01000007.1 | 3 | EZR97101.1 | 14781 |
| 690 | GCA_000683295.1 | 1422683 | 1280 | Staphylococcus aureus VET0472R | VET0472R | Firmicutes | JIKK01000013.1 | 3 | EZR91808.1 | 14784 |
| 691 | GCA_000683315.1 | 1422682 | 1280 | Staphylococcus aureus VET0471R | VET0471R | Firmicutes | JIKL01000010.1 | 3 | EZS01238.1 | 14787 |
| 692 | GCA_000683335.1 | 1422680 | 1280 | Staphylococcus aureus VET0469R | VET0469R | Firmicutes | JIKN01000014.1 | 3 | EZR98553.1 | 14790 |
| 693 | GCA_000683355.1 | 1422678 | 1280 | Staphylococcus aureus VET0467R | VET0467R | Firmicutes | JIKP01000012.1 | 3 | EZS03613.1 | 14793 |
| 694 | GCA_000683375.1 | 1422659 | 1280 | Staphylococcus aureus VET0437R | VET0437R | Firmicutes | JILI01000022.1 | 3 | EZS03317.1 | 14796 |
| 695 | GCA_000683395.1 | 1422658 | 1280 | Staphylococcus aureus VET0436R | VET0436R | Firmicutes | JILJ01000008.1 | 3 | EZS19480.1 | 14799 |
| 696 | GCA_000683415.1 | 1422650 | 1280 | Staphylococcus aureus VET0426R | VET0426R | Firmicutes | JILR01000018.1 | 3 | EZS08904.1 | 14802 |
| 697 | GCA_000683435.1 | 1422648 | 1280 | Staphylococcus aureus VET0424R | VET0424R | Firmicutes | JILT01000015.1 | 3 | EZS11744.1 | 14805 |
| 698 | GCA_000683455.1 | 1422646 | 1280 | Staphylococcus aureus VET0422R | VET0422R | Firmicutes | JILV01000006.1 | 3 | EZS15735.1 | 14808 |
| 699 | GCA_000683475.1 | 1422641 | 1280 | Staphylococcus aureus VET0414R | VET0414R | Firmicutes | JIMA01000009.1 | 3 | EZS17716.1 | 14811 |
| 700 | GCA_000683495.1 | 1422631 | 1280 | Staphylococcus aureus VET0400R | VET0400R | Firmicutes | JIMK01000012.1 | 3 | EZS22505.1 | 14814 |
| 701 | GCA_000683515.1 | 1422615 | 1280 | Staphylococcus aureus VET0368R | VET0368R | Firmicutes | JINA01000007.1 | 3 | EZS28196.1 | 14817 |
| 702 | GCA_000683535.1 | 1422610 | 1280 | Staphylococcus aureus VET0361R | VET0361R | Firmicutes | JINE01000013.1 | 3 | EZS23872.1 | 14820 |
| 703 | GCA_000683555.1 | 1422607 | 1280 | Staphylococcus aureus VET0358R | VET0358R | Firmicutes | JINH01000013.1 | 3 | EZS29422.1 | 14823 |
| 704 | GCA_000683575.1 | 1422604 | 1280 | Staphylococcus aureus VET0353R | VET0353R | Firmicutes | JINK01000012.1 | 3 | EZS31414.1 | 14826 |
| 705 | GCA_000683595.1 | 1422602 | 1280 | Staphylococcus aureus VET0343R | VET0343R | Firmicutes | JINM01000020.1 | 3 | EZS33213.1 | 14829 |
| 706 | GCA_000683615.1 | 1422601 | 1280 | Staphylococcus aureus VET0342R | VET0342R | Firmicutes | JINN01000024.1 | 3 | EZS35185.1 | 14832 |
| 707 | GCA_000683635.1 | 1422592 | 1280 | Staphylococcus aureus VET0332R | VET0332R | Firmicutes | JINW01000011.1 | 3 | EZS39162.1 | 14835 |
| 708 | GCA_000683655.1 | 1422584 | 1280 | Staphylococcus aureus VET0319R | VET0319R | Firmicutes | JIOE01000008.1 | 3 | EZS43277.1 | 14838 |
| 709 | GCA_000683675.1 | 1422573 | 1280 | Staphylococcus aureus VET0306R | VET0306R | Firmicutes | JIOP01000015.1 | 3 | EZS45538.1 | 14841 |
| 710 | GCA_000683695.1 | 1422568 | 1280 | Staphylococcus aureus VET0301R | VET0301R | Firmicutes | JIOU01000001.1 | 3 | EZS50847.1 | 14844 |
| 711 | GCA_000683715.1 | 1422565 | 1280 | Staphylococcus aureus VET0298R | VET0298R | Firmicutes | JIOX01000011.1 | 3 | EZS49647.1 | 14847 |
| 712 | GCA_000683735.1 | 1422560 | 1280 | Staphylococcus aureus VET0293R | VET0293R | Firmicutes | JIPC01000010.1 | 3 | EZS53448.1 | 14850 |
| 713 | GCA_000683755.1 | 1422532 | 1280 | Staphylococcus aureus VET0251R | VET0251R | Firmicutes | JIQE01000003.1 | 3 | EZS57832.1 | 14853 |
| 714 | GCA_000683775.1 | 1422529 | 1280 | Staphylococcus aureus VET0244R | VET0244R | Firmicutes | JIQH01000007.1 | 3 | EZS59105.1 | 14856 |
| 715 | GCA_000683795.1 | 1422524 | 1280 | Staphylococcus aureus VET0235R | VET0235R | Firmicutes | JIQM01000010.1 | 3 | EZS59928.1 | 14859 |
| 716 | GCA_000683815.1 | 1422523 | 1280 | Staphylococcus aureus VET0232R | VET0232R | Firmicutes | JIQN01000013.1 | 3 | EZS62658.1 | 14862 |
| 717 | GCA_000683835.1 | 1422521 | 1280 | Staphylococcus aureus VET0229R | VET0229R | Firmicutes | JIQP01000009.1 | 3 | EZS64792.1 | 14865 |
| 718 | GCA_000683855.1 | 1422515 | 1280 | Staphylococcus aureus VET0219R | VET0219R | Firmicutes | JIQV01000013.1 | 3 | EZS69843.1 | 14868 |
| 719 | GCA_000683875.1 | 1422503 | 1280 | Staphylococcus aureus VET0203R | VET0203R | Firmicutes | JIRD01000012.1 | 3 | EZS69138.1 | 14871 |
| 720 | GCA_000683895.1 | 1422497 | 1280 | Staphylococcus aureus VET0197R | VET0197R | Firmicutes | JIRG01000010.1 | 3 | EZS72362.1 | 14874 |
| 721 | GCA_000683915.1 | 1422491 | 1280 | Staphylococcus aureus VET0191R | VET0191R | Firmicutes | JIRL01000031.1 | 3 | EZS75507.1 | 14877 |
| 722 | GCA_000683935.1 | 1422485 | 1280 | Staphylococcus aureus VET0184R | VET0184R | Firmicutes | JIRP01000014.1 | 3 | EZS76928.1 | 14880 |
| 723 | GCA_000683955.1 | 1422484 | 1280 | Staphylococcus aureus VET0183R | VET0183R | Firmicutes | JIRQ01000012.1 | 3 | EZS80907.1 | 14883 |
| 724 | GCA_000683975.1 | 1422471 | 1280 | Staphylococcus aureus VET0159R | VET0159R | Firmicutes | JISD01000013.1 | 3 | EZS87845.1 | 14886 |
| 725 | GCA_000683995.1 | 1422470 | 1280 | Staphylococcus aureus VET0158R | VET0158R | Firmicutes | JISE01000012.1 | 3 | EZS84438.1 | 14889 |
| 726 | GCA_000684015.1 | 1422469 | 1280 | Staphylococcus aureus VET0157R | VET0157R | Firmicutes | JISF01000014.1 | 3 | EZS87769.1 | 14892 |
| 727 | GCA_000684035.1 | 1422466 | 1280 | Staphylococcus aureus VET0154R | VET0154R | Firmicutes | JISI01000011.1 | 3 | EZS90163.1 | 14895 |
| 728 | GCA_000684055.1 | 1422463 | 1280 | Staphylococcus aureus VET0150R | VET0150R | Firmicutes | JISL01000013.1 | 3 | EZS94702.1 | 14898 |
| 729 | GCA_000684075.1 | 1422461 | 1280 | Staphylococcus aureus VET0141R | VET0141R | Firmicutes | JISN01000008.1 | 3 | EZS98281.1 | 14901 |
| 730 | GCA_000684095.1 | 1422459 | 1280 | Staphylococcus aureus VET0136R | VET0136R | Firmicutes | JISP01000015.1 | 3 | EZT00071.1 | 14904 |
| 731 | GCA_000684115.1 | 1422450 | 1280 | Staphylococcus aureus VET0125R | VET0125R | Firmicutes | JISY01000005.1 | 3 | EZT04301.1 | 14907 |
| 732 | GCA_000684135.1 | 1422447 | 1280 | Staphylococcus aureus VET0120R | VET0120R | Firmicutes | JITB01000012.1 | 3 | EZT04247.1 | 14910 |
| 733 | GCA_000684155.1 | 1422441 | 1280 | Staphylococcus aureus VET0113R | VET0113R | Firmicutes | JITH01000016.1 | 3 | EZT06690.1 | 14913 |
| 734 | GCA_000684175.1 | 1422439 | 1280 | Staphylococcus aureus VET0111R | VET0111R | Firmicutes | JITJ01000011.1 | 3 | EZT09552.1 | 14916 |
| 735 | GCA_000684195.1 | 1422433 | 1280 | Staphylococcus aureus VET0102R | VET0102R | Firmicutes | JITP01000023.1 | 3 | EZT09961.1 | 14919 |
| 736 | GCA_000684215.1 | 1422415 | 1280 | Staphylococcus aureus VET0081R | VET0081R | Firmicutes | JIUH01000008.1 | 3 | EZT16280.1 | 14922 |
| 737 | GCA_000684235.1 | 1422413 | 1280 | Staphylococcus aureus VET0078R | VET0078R | Firmicutes | JIUJ01000012.1 | 3 | EZT18988.1 | 14925 |
| 738 | GCA_000684255.1 | 1422397 | 1280 | Staphylococcus aureus VET0058R | VET0058R | Firmicutes | JIUZ01000012.1 | 3 | EZT18347.1 | 14928 |
| 739 | GCA_000684275.1 | 1422390 | 1280 | Staphylococcus aureus VET0050R | VET0050R | Firmicutes | JIVG01000012.1 | 3 | EZT23523.1 | 14931 |
| 740 | GCA_000684295.1 | 1413450 | 1280 | Staphylococcus aureus VE08/02242ST1 | VE08/02242ST1 | Firmicutes | JIVI01000014.1 | 3 | EZT25332.1 | 14934 |
| 741 | GCA_000684315.1 | 1413603 | 1280 | Staphylococcus aureus USA_6 | USA_6 | Firmicutes | JIV001000013.1 | 3 | EZT27375.1 | 14937 |
| 742 | GCA_000684335.1 | 1413566 | 1280 | Staphylococcus aureus Tur-5 | Tur-5 | Firmicutes | JIVW01000016.1 | 3 | EZT32068.1 | 14940 |
| 743 | GCA_000684355.1 | 1413553 | 1280 | Staphylococcus aureus Tur-4 | Tur-4 | Firmicutes | JIVX01000016.1 | 3 | EZT32807.1 | 14943 |
| 744 | GCA_000684375.1 | 1413487 | 1280 | Staphylococcus aureus S64_POEL | S64_POEL | Firmicutes | JIWP01000013.1 | 3 | EZT35387.1 | 14946 |
| 745 | GCA_000684395.1 | 1413486 | 1280 | Staphylococcus aureus S63_POEL | S63_POEL | Firmicutes | JIWQ01000014.1 | 3 | EZT42046.1 | 14949 |
| 746 | GCA_000684415.1 | 1413565 | 1280 | Staphylococcus aureus Rd.290 | Rd.290 | Firmicutes | JIXA01000010.1 | 3 | EZT39172.1 | 14952 |
| 747 | GCA_000684435.1 | 1413494 | 1280 | Staphylococcus aureus MSSA-47 | MSSA-47 | Firmicutes | JIX001000015.1 | 3 | EZT44508.1 | 14955 |
| 748 | GCA_000684455.1 | 1413493 | 1280 | Staphylococcus aureus MSSA-37 | MSSA-37 | Firmicutes | JIXP01000012.1 | 3 | EZT47451.1 | 14958 |
| 749 | GCA_000684475.1 | 1413496 | 1280 | Staphylococcus aureus MSSA-123 | MSSA-123 | Firmicutes | JIXQ01000009.1 | 3 | EZT50536.1 | 14961 |
| 750 | GCA_000684495.1 | 1413536 | 1280 | Staphylococcus aureus GD2010-169 | GD2010-169 | Firmicutes | JIXZ01000015.1 | 3 | EZT52625.1 | 14964 |
| 751 | GCA_000684515.1 | 1413535 | 1280 | Staphylococcus aureus GD2010-159 | GD2010-159 | Firmicutes | JIYB01000010.1 | 3 | EZT54280.1 | 14967 |
| 752 | GCA_000684535.1 | 1413473 | 1280 | Staphylococcus aureus C3452 | C3452 | Firmicutes | JIZF01000012.1 | 3 | EZT57811.1 | 14970 |
| 753 | GCA_000684555.1 | 1413511 | 1280 | Staphylococcus aureus C0676 | C0676 | Firmicutes | JIZR01000010.1 | 3 | EZT60957.1 | 14973 |
| 754 | GCA_000684575.1 | 1413507 | 1280 | Staphylococcus aureus C0626 | C0626 | Firmicutes | JIZV01000007.1 | 3 | EZT61426.1 | 14976 |
| 755 | GCA_000684595.1 | 1413325 | 1280 | Staphylococcus aureus 81629 | 81629 | Firmicutes | JJAH01000015.1 | 3 | EZT65757.1 | 14979 |
| 756 | GCA_000684615.1 | 1413343 | 1280 | Staphylococcus aureus 63-D10 | 63-D10 | Firmicutes | JJAN01000014.1 | 3 | EZT68345.1 | 14982 |
| 757 | GCA_000684635.1 | 1413388 | 1280 | Staphylococcus aureus 53180-1 | 53180-1 | Firmicutes | JJAW01000011.1 | 3 | EZT68418.1 | 14985 |
| 758 | GCA_000684655.1 | 1413547 | 1280 | Staphylococcus aureus 45(2607) | 45(2607) | Firmicutes | JJAZ01000013.1 | 3 | EZT73755.1 | 14988 |
| 759 | GCA_000684675.1 | 1413544 | 1280 | Staphylococcus aureus 28(3K2-5) | 28(3K2-5) | Firmicutes | JJBI01000015.1 | 3 | EZT75322.1 | 14991 |
| 760 | GCA_000684695.1 | 1413543 | 1280 | Staphylococcus aureus 22(2K81-5) | 22(2K81-5) | Firmicutes | JJBX01000016.1 | 3 | EZT77630.1 | 14994 |
| 761 | GCA_000684715.1 | 1413522 | 1280 | Staphylococcus aureus 2011-60-2275-7 | 2011-60-2275-7 | Firmicutes | JJBY01000031.1 | 3 | EZT80087.1 | 14997 |
| 762 | GCA_000684735.1 | 1413394 | 1280 | Staphylococcus aureus 1484-9 | 1484-9 | Firmicutes | JJCS01000014.1 | 3 | EZT82073.1 | 15000 |
| 763 | GCA_000684795.1 | 1413430 | 1280 | Staphylococcus aureus 1111001578 | 1111001578 | Firmicutes | JJDU01000014.1 | 3 | EZT89290.1 | 15003 |
| 764 | GCA_000684815.1 | 1413357 | 1280 | Staphylococcus aureus 10S00488 | 10S00488 | Firmicutes | JJEI01000012.1 | 3 | EZT93146.1 | 15006 |
| 765 | GCA_000684835.1 | 1413560 | 1280 | Staphylococcus aureus 1(04GN_04-02-52-07) | 1(04GN_04-02-52-07) | Firmicutes | JJEJ01000010.1 | 3 | EZT96081.1 | 15009 |
| 766 | GCA_000684855.1 | 1413356 | 1280 | Staphylococcus aureus 09S01694 | 09S01694 | Firmicutes | JJEK01000015.1 | 3 | EZT99582.1 | 15012 |
| 767 | GCA_000684875.1 | 1413349 | 1280 | Staphylococcus aureus 09-00736 | 09-00736 | Firmicutes | JJEM01000015.1 | 3 | EZU00841.1 | 15015 |
| 768 | GCA_000684895.1 | 1413346 | 1280 | Staphylococcus aureus 08-01229 | 08-01229 | Firmicutes | JJET01000013.1 | 3 | EZU03294.1 | 15018 |
| 769 | GCA_000684915.1 | 1413345 | 1280 | Staphylococcus aureus 08-01062 | 08-01062 | Firmicutes | JJEW01000012.1 | 3 | EZU07140.1 | 15021 |
| 770 | GCA_000684955.1 | 1413344 | 1280 | Staphylococcus aureus 08-01059 | 08-01059 | Firmicutes | JJEX01000015.1 | 3 | EZU08872.1 | 15024 |
| 771 | GCA_000627945.1 | 1444105 | 562 | Escherichia coli 1-176-05_S1_C3 | 1-176-05_S1_C3 | Proteobacteria | JJLY01000082.1 | 2 | EZK05852.1 | 15027 |
| 772 | GCA_000627905.1 | 1444078 | 562 | Escherichia coli 1-176-05_S1_C2 | 1-176-05_S1_C2 | Proteobacteria | JJMA01000042.1 | 2 | EZK20524.1 | 15029 |
| 773 | GCA_000705535.1 | 95486 | 95486 | Burkholderia cenocepacia | 869T2 | Proteobacteria | JJOA01000083.1 | 3 | KEA54804.1 | 15031 |
| 774 | GCA_000764325.1 | 29495 | 29495 | Vibrio navarrensis | ATCC 51183 | Proteobacteria | JMCG01000001.1 | 2 | KGK10824.1 | 15034 |
| 775 | GCA_000695795.1 | 1492738 | 1492738 | Flavobacterium seoulense | EM1321 | Bacteroidetes | JNCA01000003.1 | 3 | KDN56466.1 | 15036 |
| 776 | GCA_000699865.1 | 1339352 | 821 | Bacteroides vulgatus str. 3975 RP4 | 3975 RP4 | Bacteroidetes | JNHM01000174.1 | 3 | KDS43697.1 | 15039 |
| 777 | GCA_000700705.1 | 1444060 | 562 | Escherichia coli 4-203-08_S1_C1 | 4-203-08_S1_C1 | Proteobacteria | JNMY01000013.1 | 2 | KDU61955.1 | 15042 |
| 778 | GCA_000704125.1 | 1444070 | 562 | Escherichia coli 2-222-05_S1_C2 | 2-222-05_S1_C2 | Proteobacteria | JNQL01000002.1 | 2 | KDX77544.1 | 15044 |
| 779 | GCA_000704285.1 | 1444098 | 562 | Escherichia coli 2-222-05_S1_C3 | 2-222-05_S1_C3 | Proteobacteria | JNQM01000001.1 | 2 | KDX82612.1 | 15046 |
| 780 | GCA_000704405.1 | 1444183 | 562 | Escherichia coli 2-427-07_S3_C3 | 2-427-07_S3_C3 | Proteobacteria | JNQY01000031.1 | 2 | KDY27416.1 | 15048 |
| 781 | GCA_000707425.1 | 670 | 670 | Vibrio parahaemolyticus | CFSAN007430 | Proteobacteria | JNSN01000106.1 | 2 | KED12527.1 | 15050 |
| 782 | GCA_000707385.1 | 670 | 670 | Vibrio parahaemolyticus | CFSAN007431 | Proteobacteria | JNSO01000031.1 | 2 | KED15841.1 | 15052 |
| 783 | GCA_000707465.1 | 670 | 670 | Vibrio parahaemolyticus | CFSAN007433 | Proteobacteria | JNSQ01000049.1 | 3 | KED29323.1 | 15054 |
| 784 | GCA_000707485.1 | 670 | 670 | Vibrio parahaemolyticus | CFSAN007434 | Proteobacteria | JNSR01000049.1 | 3 | KED25553.1 | 15057 |
| 785 | GCA_000707505.1 | 670 | 670 | Vibrio parahaemolyticus | CFSAN007435 | Proteobacteria | JNSS01000023.1 | 3 | KED36896.1 | 15060 |
| 786 | GCA_000706885.1 | 670 | 670 | Vibrio parahaemolyticus | CFSAN007436 | Proteobacteria | JNST01000065.1 | 2 | KED34398.1 | 15063 |
| 787 | GCA_000707685.1 | 670 | 670 | Vibrio parahaemolyticus | CFSAN007446 | Proteobacteria | JNTD01000023.1 | 2 | KED69849.1 | 15065 |
| 788 | GCA_000764405.1 | 70775 | 70775 | Pseudomonas plecoglossicida | TND35 | Proteobacteria | JOJY01000001.1 | 3 | KGK23635.1 | 15067 |
| 789 | GCA_000713945.1 | 1444088 | 562 | Escherichia coli 4-203-08_S1_C2 | 4-203-08_S1_C2 | Proteobacteria | JOQF01000029.1 | 2 | KEK91460.1 | 15070 |
| 790 | GCA_000713975.1 | 1444113 | 562 | Escherichia coli 4-203-08_S1_C3 | 4-203-08_S1_C3 | Proteobacteria | JOQG01000030.1 | 2 | KEK94906.1 | 15072 |
| 791 | GCA_000713975.1 | 1444113 | 562 | Escherichia coli 4-203-08_S1_C3 | 4-203-08_S1_C3 | Proteobacteria | JOQG01000030.1 | 2 | KEK94966.1 | 15074 |
| 792 | GCA_000713975.1 | 1444113 | 562 | Escherichia coli 4-203-08_S1_C3 | 4-203-08_S1_C3 | Proteobacteria | JOQG01000115.1 | 2 | KEK89446.1 | 15076 |
| 793 | GCA_000722685.1 | 28037 | 28037 | Streptococcus mitis | SK667 | Firmicutes | JPFV01000011.1 | 3 | KEQ37439.1 | 15078 |
| 794 | GCA_000722755.1 | 28037 | 28037 | Streptococcus mitis | SK578 | Firmicutes | JPFY01000013.1 | 3 | KEQ45048.1 | 15081 |
| 795 | GCA_000734475.1 | 615 | 615 | Serratia marcescens | YDC563 | Proteobacteria | JPOB01000015.1 | 2 | KFB55438.1 | 15084 |
| 796 | GCA_000738535.1 | 615 | 615 | Serratia marcescens | MCB | Proteobacteria | JPQY01000010.1 | 2 | KFF79466.1 | 15086 |
| 797 | GCA_000738105.1 | 180957 | 554 | Pectobacterium carotovorum subsp. brasiliense | CFIA1009 | Proteobacteria | JPSN01000039.1 | 5 | KFF62084.1 | 15088 |
| 798 | GCA_001466295.1 | 1639 | 1639 | Listeria monocytogenes | G4599 | Firmicutes | JPTX01000012.1 | 2 | KTA63996.1 | 15093 |
| 799 | GCA_001190115.1 | 1089447 | 714 | Aggregatibacter actinomycetemcomitans | RHAA1 | Proteobacteria | JPZI01000001.1 | 2 | KNE78570.1 | 15095 |
| | | | | RhAA1 | | | | | | |
| 800 | GCA_000743165.1 | 672 | 672 | Vibrio vulnificus | 2322 | Proteobacteria | JQDS01000004.1 | 3 | KFK51887.1 | 15097 |
| 801 | GCA_000754695.1 | 180957 | 554 | Pectobacterium carotovorum subsp. brasiliense | LMG 21371 | Proteobacteria | JQOE01000001.1 | 2 | KGA25874.1 | 15100 |
| 802 | GCA_000768895.1 | 1515614 | 1515614 | Porphyromonas sp. COT-108 OH2963 | COT-108 OH2963 | Bacteroidetes | JRAP01000021.1 | 2 | KGN93833.1 | 15102 |
| 803 | GCA_000764975.1 | 1352 | 1352 | Enterococcus faecium | UC7266 | Firmicutes | JRJW01000071.1 | 3 | KGK75437.1 | 15104 |
| 804 | GCA_000759165.1 | 1401072 | 28125 | Prevotella bivia DNF00650 | DNF00650 | Bacteroidetes | JRNM01000003.1 | 3 | KGF38953.1 | 15107 |
| 805 | GCA_000785785.1 | 1349 | 1349 | Streptococcus uberis | CAIM 1894 | Firmicutes | JRQN01000035.1 | 2 | KHD43839.1 | 15110 |
| 806 | GCA_000772505.1 | 1352 | 1352 | Enterococcus faecium | VRE3 | Firmicutes | JSET01000005.1 | 3 | KGQ80278.1 | 15112 |
| 807 | GCA_000817445.1 | 715 | 715 | Actinobacillus pleuropneumoniae | 1022 | Proteobacteria | JSVF01000019.1 | 3 | KIE90685.1 | 15115 |
| 808 | GCA_000817465.1 | 715 | 715 | Actinobacillus pleuropneumoniae | 460 | Proteobacteria | JSVG01000021.1 | 3 | KIE90602.1 | 15118 |
| 809 | GCA_000817515.1 | 715 | 715 | Actinobacillus pleuropneumoniae | 780 | Proteobacteria | JSVV01000021.1 | 3 | KIE97285.1 | 15121 |
| 810 | GCA_000817535.1 | 715 | 715 | Actinobacillus pleuropneumoniae | 597 | Proteobacteria | JSVX01000022.1 | 3 | KIE97672.1 | 15124 |
| 811 | GCA_000817525.1 | 715 | 715 | Actinobacillus pleuropneumoniae | 5651 | Proteobacteria | JSVY01000018.1 | 3 | KIE96199.1 | 15127 |
| 812 | GCA_000817485.1 | 715 | 715 | Actinobacillus pleuropneumoniae | 518 | Proteobacteria | JSVZ01000017.1 | 3 | KIE90357.1 | 15130 |
| 813 | GCA_000803215.1 | 554 | 554 | Pectobacterium carotovorum | M022 | Proteobacteria | JSXC01000002.1 | 5 | KHN56276.1 | 15133 |
| 814 | GCA_000827155.1 | 1280 | 1280 | Staphylococcus aureus | K12PJN53 | Firmicutes | JSYA01000018.1 | 3 | KII20612.1 | 15138 |
| 815 | GCA_000817375.1 | 562 | 562 | Escherichia coli | SCB11 | Proteobacteria | JSYT01000001.1 | 2 | KIE70777.1 | 15141 |
| 816 | GCA_000783635.1 | 644 | 644 | Aeromonas hydrophila | FDAARGOS_78 | Proteobacteria | JTBD01000005.1 | 2 | KGY52609.1 | 15143 |
| 817 | GCA_000783915.1 | 615 | 615 | Serratia marcescens | FDAARGOS_65 | Proteobacteria | JTBR01000001.1 | 2 | KGZ13596.1 | 15145 |
| 818 | GCA_000803085.1 | 1315273 | 137779 | Pseudoalteromonas flavipulchra NCIMB | ATCC BAA-314 | Proteobacteria | JTDZ01000035.1 | 2 | KHM45375.1 | 15147 |
| | | | | 2033 = ATCC BAA-314 | | | | | | |
| 819 | GCA_000814675.1 | 77608 | 77608 | Pseudoalteromonas distincta | ATCC 700518 | Proteobacteria | JWIG01000011.1 | 2 | KID39107.1 | 15149 |
| 820 | GCA_000805875.1 | 615 | 615 | Serratia marcescens | RM66262 | Proteobacteria | JWL001000004.1 | 2 | KHO41399.1 | 15151 |
| 821 | GCA_000830335.1 | 40479 | 2095 | Mycoplasma capricolum subsp. capricolum | GM508D | Tenericutes | JXQB01000001.1 | 2 | KIM13727.1 | 15153 |
| 822 | GCA_001042915.1 | 47884 | 47884 | Pseudomonas taetrolens | DSM 21104 | Proteobacteria | JYLA01000003.1 | 2 | KMM85341.1 | 15155 |
| 823 | GCA_000321685.1 | 1138891 | 1352 | Enterococcus faecium EnGen0011 | E1185 | Firmicutes | KB029511.1 | 3 | ELA78317.1 | 15157 |
| 824 | GCA_000322365.1 | 1138931 | 1352 | Enterococcus faecium EnGen0051 | E1644 | Firmicutes | KB030041.1 | 3 | ELB72564.1 | 15160 |
| 825 | GCA_000322385.1 | 1138932 | 1352 | Enterococcus faecium EnGen0050 | E2369 | Firmicutes | KB030078.1 | 3 | ELB70911.1 | 15163 |
| 826 | GCA_000322405.1 | 1138933 | 1352 | Enterococcus faecium EnGen0046 | E2560 | Firmicutes | KB030094.1 | 3 | ELB76087.1 | 15166 |
| 827 | GCA_000322465.1 | 1138936 | 1352 | Enterococcus faecium EnGen0049 | E6045 | Firmicutes | KB030126.1 | 3 | ELB84571.1 | 15169 |
| 828 | GCA_000322445.1 | 1138935 | 1352 | Enterococcus faecium EnGen0045 | E6012 | Firmicutes | KB030159.1 | 3 | ELB85483.1 | 15172 |
| 829 | GCA_000314675.2 | 883078 | 56946 | Afipia broomeae ATCC 49717 | ATCC 49717 | Proteobacteria | KB375284.1 | 2 | EKS34044.1 | 15175 |
| 830 | GCA_000350765.1 | 1169335 | 562 | Escherichia coli KTE16 | KTE16 | Proteobacteria | KB732288.1 | 2 | ELC28356.1 | 15177 |
| 831 | GCA_000351265.1 | 1181770 | 562 | Escherichia coli KTE224 | KTE224 | Proteobacteria | KB732567.1 | 2 | ELD53295.1 | 15179 |
| 832 | GCA_000352025.1 | 1182736 | 562 | Escherichia coli KTE169 | KTE169 | Proteobacteria | KB732976.1 | 2 | ELF38886.1 | 15181 |
| 833 | GCA_000352185.1 | 1169359 | 562 | Escherichia coli KTE42 | KTE42 | Proteobacteria | KB733011.1 | 2 | ELF73880.1 | 15183 |
| 834 | GCA_000352805.1 | 1181740 | 562 | Escherichia coli KTE194 | KTE194 | Proteobacteria | KB733150.1 | 2 | ELH15113.1 | 15185 |
| 835 | GCA_000352225.1 | 1169360 | 562 | Escherichia coli KTE43 | KTE43 | Proteobacteria | KB733426.1 | 2 | ELF82585.1 | 15187 |
| 836 | GCA_000368165.1 | 1217669 | 28090 | Acinetobacter Iwoffii NIPH 715 | NIPH 715 | Proteobacteria | KB849267.1 | 3 | ENU62565.1 | 15189 |
| 837 | GCA_000369545.1 | 1217702 | 1217702 | Acinetobacter sp. NIPH 1867 | NIPH 1867 | Proteobacteria | KB850085.1 | 3 | ENX56755.1 | 15192 |
| 838 | GCA_000369685.1 | 1144665 | 1144665 | Acinetobacter sp. CIP 102136 | CIP 102136 | Proteobacteria | KB850142.1 | 2 | ENX19931.1 | 15195 |
| 839 | GCA_000369725.1 | 1144662 | 1144662 | Acinetobacter sp. CIP 101966 | CIP 101966 | Proteobacteria | KB850160.1 | 3 | ENX27881.1 | 15197 |
| 840 | GCA_000371465.1 | 999411 | 179628 | Clostridium colicanis 209318 | 209318 | Firmicutes | KB850956.1 | 3 | ENZ03437.1 | 15200 |
| 841 | GCA_000371465.1 | 999411 | 179628 | Clostridium colicanis 209318 | 209318 | Firmicutes | KB850956.1 | 3 | ENZ02555.1 | 15203 |
| 842 | GCA_000393775.1 | 1158555 | 1352 | Enterococcus faecium EnGen0265 | UAA1484 | Firmicutes | KB946149.1 | 3 | EOH58476.1 | 15206 |
| 843 | GCA_000394575.1 | 1158644 | 1352 | Enterococcus faecium EnGen0320 | UAA949 | Firmicutes | KB946824.1 | 3 | EOL74786.1 | 15209 |
| 844 | GCA_000394595.1 | 1158645 | 1352 | Enterococcus faecium EnGen0322 | UAA950 | Firmicutes | KB946830.1 | 3 | EOI52382.1 | 15212 |
| 845 | GCA_000395645.1 | 1157463 | 1352 | Enterococcus faecium EnGen0168 | UAA725 | Firmicutes | KB947813.1 | 3 | EOG00796.1 | 15215 |
| 846 | GCA_000395665.1 | 1157464 | 1352 | Enterococcus faecium EnGen0169 | UAA825 | Firmicutes | KB947831.1 | 3 | EOG00999.1 | 15218 |
| 847 | GCA_000395825.1 | 1157472 | 1352 | Enterococcus faecium EnGen0177 | VRE 108 | Firmicutes | KB948035.1 | 3 | EOG17544.1 | 15221 |
| 848 | GCA_000395865.1 | 1157474 | 1352 | Enterococcus faecium EnGen0179 | VRE 13 | Firmicutes | KB948120.1 | 3 | EOG17727.1 | 15224 |
| 849 | GCA_000395885.1 | 1157475 | 1352 | Enterococcus faecium EnGen0180 | VRE 84 | Firmicutes | KB948121.1 | 3 | EOG29141.1 | 15227 |
| 850 | GCA_000396925.1 | 1158543 | 1352 | Enterococcus faecium EnGen0255 | UAA951 | Firmicutes | KB949696.1 | 3 | EOM31821.1 | 15230 |
| 851 | GCA_000399545.1 | 1053235 | 1396 | Bacillus cereus VD140 | VD140 | Firmicutes | KB976803.1 | 2 | EOQ01914.1 | 15233 |
| 852 | GCA_000406925.1 | 1121865 | 1355 | Enterococcus columbae DSM 7374 = | ATCC 51263 | Firmicutes | KE136347.1 | 2 | EOT43559.1 | 15235 |
| | | | | ATCC 51263 | | | | | | |
| 853 | GCA_000407225.1 | 1121865 | 1355 | Enterococcus columbae DSM 7374 = | ATCC 51263 | Firmicutes | KE136492.1 | 2 | EOW87387.1 | 15237 |
| | | | | ATCC 51263 | | | | | | |
| 854 | GCA_000411115.1 | 641143 | 45242 | Capnocytophaga granulosa ATCC 51502 | ATCC 51502 | Bacteroidetes | KE150244.1 | 4 | EPD26424.1 | 15239 |
| 855 | GCA_000415365.1 | 1244150 | 1352 | Enterococcus faecium LA4B-2 | LA4B-2 | Firmicutes | KE352415.1 | 2 | EPI22405.1 | 15243 |
| 856 | GCA_000456145.1 | 1280963 | 562 | Escherichia coli HVH 9 (4-6942539) | HVH 9 (4-6942539) | Proteobacteria | KE698882.1 | 2 | EQN31627.1 | 15245 |
| 857 | GCA_000456585.1 | 1280988 | 562 | Escherichia coli HVH 38 (4-2774682) | HVH 38 (4-2774682) | Proteobacteria | KE699187.1 | 2 | EQO47878.1 | 15247 |
| 858 | GCA_000456745.1 | 1280996 | 562 | Escherichia coli HVH 46 (4-2758776) | HVH 46 (4-2758776) | Proteobacteria | KE699286.1 | 2 | EQO88544.1 | 15249 |
| 859 | GCA_000458275.1 | 1281086 | 562 | Escherichia coli HVH 153 (3-9344314) | HVH 153 (3-9344314) | Proteobacteria | KE700288.1 | 2 | EQS52095.1 | 15251 |
| 860 | GCA_000460435.1 | 1281198 | 562 | Escherichia coli UMEA 3160-1 | UMEA 3160-1 | Proteobacteria | KE701742.1 | 2 | EQX21241.1 | 15253 |
| 861 | GCA_000469405.1 | 888055 | 157687 | Leptotrichia wadei F0279 | F0279 | Fusobacteria | KI271427.1 | 2 | ERK54360.1 | 15255 |
| 862 | GCA_000481425.1 | 1402514 | 287 | Pseudomonas aeruginosa BWHPSA014 | BWHPSA014 | Proteobacteria | KI519076.1 | 3 | ERW54756.1 | 15257 |
| 863 | GCA_000481625.1 | 1402504 | 287 | Pseudomonas aeruginosa BWHPSA004 | BWHPSA004 | Proteobacteria | KI519157.1 | 3 | ERW94009.1 | 15260 |
| 864 | GCA_000507745.1 | 1073373 | 1305 | Streptococcus sanguinis CC94A | CC94A | Firmicutes | KI669394.1 | 3 | ETD07628.1 | 15263 |
| 865 | GCA_000519605.1 | 1391479 | 1351 | Enterococcus faecalis EnGen0409 | EnGen0409 | Firmicutes | KI912997.1 | 3 | ETU12932.1 | 15266 |
| 866 | GCA_000521925.1 | 1400186 | 615 | Serratia marcescens BIDMC 44 | BIDMC 44 | Proteobacteria | KI929263.1 | 2 | ETX46915.1 | 15269 |
| 867 | GCA_000599425.1 | 1348563 | 28901 | Salmonella enterica subsp. enterica serovar | 05-2929 | Proteobacteria | KK082454.1 | 2 | EXX84012.1 | 15271 |
| | | | | Namur str. 05-2929 | | | | | | |
| 868 | GCA_000633715.1 | 1445864 | 615 | Serratia marcescens BIDMC 80 | BIDMC 80 | Proteobacteria | KK214322.1 | 2 | EZQ69339.1 | 15273 |
| 869 | GCA_000636175.1 | 1413352 | 1280 | Staphylococcus aureus 08-01084 | 08-01084 | Firmicutes | KK218077.1 | 3 | EZU15808.1 | 15275 |
| 870 | GCA_000636195.1 | 1413353 | 1280 | Staphylococcus aureus 08-01085 | 08-01085 | Firmicutes | KK218112.1 | 3 | EZU14925.1 | 15278 |
| 871 | GCA_000636215.1 | 1413347 | 1280 | Staphylococcus aureus 08-01728 | 08-01728 | Firmicutes | KK218139.1 | 3 | EZU22369.1 | 15281 |
| 872 | GCA_000636255.1 | 1413597 | 1280 | Staphylococcus aureus 08134-6 | 08134-6 | Firmicutes | KK218219.1 | 3 | EZU24877.1 | 15284 |
| 873 | GCA_000636275.1 | 1413598 | 1280 | Staphylococcus aureus 08139-6 | 08139-6 | Firmicutes | KK218244.1 | 3 | EZU30074.1 | 15287 |
| 874 | GCA_000636295.1 | 1413599 | 1280 | Staphylococcus aureus 08142-8 | 08142-8 | Firmicutes | KK218289.1 | 3 | EZU33076.1 | 15290 |
| 875 | GCA_000636315.1 | 1413600 | 1280 | Staphylococcus aureus 08143-5 | 08143-5 | Firmicutes | KK218349.1 | 3 | EZU39423.1 | 15293 |
| 876 | GCA_000636335.1 | 1413355 | 1280 | Staphylococcus aureus 09S00475 | 09S00475 | Firmicutes | KK218433.1 | 3 | EZU35477.1 | 15296 |
| 877 | GCA_000636355.1 | 1413358 | 1280 | Staphylococcus aureus 10S01493 | 10S01493 | Firmicutes | KK218470.1 | 3 | EZU41791.1 | 15299 |
| 878 | GCA_000636375.1 | 1413423 | 1280 | Staphylococcus aureus 110802495 | 110802495 | Firmicutes | KK218519.1 | 3 | EZU42124.1 | 15302 |
| 879 | GCA_000636395.1 | 1413418 | 1280 | Staphylococcus aureus 1110601704 | 1110601704 | Firmicutes | KK218547.1 | 3 | EZU46569.1 | 15305 |
| 880 | GCA_000636415.1 | 1413419 | 1280 | Staphylococcus aureus 1110601896 | 1110601896 | Firmicutes | KK218599.1 | 3 | EZU46770.1 | 15308 |
| 881 | GCA_000636435.1 | 1413420 | 1280 | Staphylococcus aureus 1110700562 | 1110700562 | Firmicutes | KK218635.1 | 3 | EZU48615.1 | 15311 |
| 882 | GCA_000636455.1 | 1413421 | 1280 | Staphylococcus aureus 1110700610 | 1110700610 | Firmicutes | KK218658.1 | 3 | EZU52018.1 | 15314 |
| 883 | GCA_000636475.1 | 1413422 | 1280 | Staphylococcus aureus 1110701127 | 1110701127 | Firmicutes | KK218690.1 | 3 | EZU55227.1 | 15317 |
| 884 | GCA_000636495.1 | 1413424 | 1280 | Staphylococcus aureus 1110802883 | 1110802883 | Firmicutes | KK218732.1 | 3 | EZU56168.1 | 15320 |
| 885 | GCA_000636535.1 | 1413426 | 1280 | Staphylococcus aureus 1110807699 | 1110807699 | Firmicutes | KK218822.1 | 3 | EZU61337.1 | 15323 |
| 886 | GCA_000636555.1 | 1413427 | 1280 | Staphylococcus aureus 1110904178 | 1110904178 | Firmicutes | KK218847.1 | 3 | EZU65300.1 | 15326 |
| 887 | GCA_000636635.1 | 1413432 | 1280 | Staphylococcus aureus 1111101949 | 1111101949 | Firmicutes | KK219058.1 | 3 | EZU73662.1 | 15329 |
| 888 | GCA_000636655.1 | 1413433 | 1280 | Staphylococcus aureus 1111102620 | 1111102620 | Firmicutes | KK219075.1 | 3 | EZU80187.1 | 15332 |
| 889 | GCA_000636675.1 | 1413434 | 1280 | Staphylococcus aureus 1111200013 | 1111200013 | Firmicutes | KK219116.1 | 3 | EZU80949.1 | 15335 |
| 890 | GCA_000636695.1 | 1413435 | 1280 | Staphylococcus aureus 1111203374 | 1111203374 | Firmicutes | KK219160.1 | 3 | EZU82981.1 | 15338 |
| 891 | GCA_000636715.1 | 1413436 | 1280 | Staphylococcus aureus 1111205429 | 1111205429 | Firmicutes | KK219204.1 | 3 | EZU87062.1 | 15341 |
| 892 | GCA_000636735.1 | 1413437 | 1280 | Staphylococcus aureus 1111206270 | 1111206270 | Firmicutes | KK219261.1 | 3 | EZU88265.1 | 15344 |
| 893 | GCA_000636755.1 | 1413587 | 1280 | Staphylococcus aureus 11P4 | 11P4 | Firmicutes | KK219305.1 | 3 | EZU90294.1 | 15347 |
| 894 | GCA_000636775.1 | 1413588 | 1280 | Staphylococcus aureus 11P8 | 11P8 | Firmicutes | KK219338.1 | 3 | EZU94768.1 | 15350 |
| 895 | GCA_000636815.1 | 1413360 | 1280 | Staphylococcus aureus 11S00627 | 11S00627 | Firmicutes | KK219434.1 | 3 | EZU96101.1 | 15353 |
| 896 | GCA_000636855.1 | 1413363 | 1280 | Staphylococcus aureus 11S01586 | 11S01586 | Firmicutes | KK219518.1 | 3 | EZV02408.1 | 15356 |
| 897 | GCA_000636895.1 | 1413491 | 1280 | Staphylococcus aureus 122 | 122 | Firmicutes | KK219580.1 | 3 | EZV09969.1 | 15359 |
| 898 | GCA_000636915.1 | 1413416 | 1280 | Staphylococcus aureus 12464 | 12464 | Firmicutes | KK219611.1 | 3 | EZV11275.1 | 15362 |
| 899 | GCA_000636955.1 | 1413365 | 1280 | Staphylococcus aureus 12S00579 | 12S00579 | Firmicutes | KK219698.1 | 3 | EZV17198.1 | 15365 |
| 900 | GCA_000636975.1 | 1413366 | 1280 | Staphylococcus aureus 12S00881 | 12S00881 | Firmicutes | KK219743.1 | 3 | EZV18375.1 | 15368 |
| 901 | GCA_000636995.1 | 1413368 | 1280 | Staphylococcus aureus 12S01153 | 12S01153 | Firmicutes | KK219769.1 | 3 | EZV22691.1 | 15371 |
| 902 | GCA_000637015.1 | 1413369 | 1280 | Staphylococcus aureus 12S01399 | 12S01399 | Firmicutes | KK219813.1 | 3 | EZV23635.1 | 15374 |
| 903 | GCA_000637055.1 | 1413371 | 1280 | Staphylococcus aureus 12-ST01988 | 12-ST01988 | Firmicutes | KK219900.1 | 3 | EZV30021.1 | 15377 |
| 904 | GCA_000637095.1 | 1413562 | 1280 | Staphylococcus aureus 14(11MN_17- | 14(11MN_17- | Firmicutes | KK219997.1 | 3 | EZV34240.1 | 15380 |
| | | | | 08-66-05) | 08-66-05) | | | | | |
| 905 | GCA_000637115.1 | 1413401 | 1280 | Staphylococcus aureus 150211/pool 1 | 150211/pool 1 | Firmicutes | KK220038.1 | 3 | EZV37879.1 | 15383 |
| 906 | GCA_000637135.1 | 1413558 | 1280 | Staphylococcus aureus 16(11S1S4-09) | 16(115154-09) | Firmicutes | KK220079.1 | 3 | EZV41499.1 | 15386 |
| 907 | GCA_000637155.1 | 1413402 | 1280 | Staphylococcus aureus 18439-17 | 18439-17 | Firmicutes | KK220114.1 | 3 | EZV40173.1 | 15389 |
| 908 | GCA_000637175.1 | 1413405 | 1280 | Staphylococcus aureus 18439-62 | 18439-62 | Firmicutes | KK220145.1 | 3 | EZV45691.1 | 15392 |
| 909 | GCA_000637215.1 | 1413333 | 1280 | Staphylococcus aureus 19571 | 19571 | Firmicutes | KK220240.1 | 3 | EZV49483.1 | 15395 |
| 910 | GCA_000637235.1 | 1413561 | 1280 | Staphylococcus aureus 2(04HN_17- | 2(04HN_17- | Firmicutes | KK220279.1 | 3 | EZV51743.1 | 15398 |
| | | | | 03-52-05) | 03-52-05) | | | | | |
| 911 | GCA_000637255.1 | 1413519 | 1280 | Staphylococcus aureus 2009-60-561-1 | 2009-60-561-1 | Firmicutes | KK220312.1 | 3 | EZV56161.1 | 15401 |
| 912 | GCA_000637275.1 | 1413514 | 1280 | Staphylococcus aureus 2010-60-1240-1 | 2010-60-1240-1 | Firmicutes | KK220355.1 | 3 | EZV60061.1 | 15404 |
| 913 | GCA_000637295.1 | 1413328 | 1280 | Staphylococcus aureus 2010-60-6063-29 | 2010-60-6063-29 | Firmicutes | KK220412.1 | 3 | EZV56179.1 | 15407 |
| 914 | GCA_000637315.1 | 1413516 | 1280 | Staphylococcus aureus 2010-60-6063-39 | 2010-60-6063-39 | Firmicutes | KK220436.1 | 3 | EZV62995.1 | 15410 |
| 915 | GCA_000637355.1 | 1413517 | 1280 | Staphylococcus aureus 2010-60-6511-39 | 2010-60-6511-39 | Firmicutes | KK220514.1 | 3 | EZV68414.1 | 15413 |
| 916 | GCA_000637395.1 | 1413329 | 1280 | Staphylococcus aureus 2010-60-7626-19 | 2010-60-7626-19 | Firmicutes | KK220613.1 | 3 | EZV73439.1 | 15416 |
| 917 | GCA_000637415.1 | 1413524 | 1280 | Staphylococcus aureus 2011-60-1490-31 | 2011-60-1490-31 | Firmicutes | KK220641.1 | 3 | EZV79302.1 | 15419 |
| 918 | GCA_000637435.1 | 1413523 | 1280 | Staphylococcus aureus 2011-60-2078-5 | 2011-60-2078-5 | Firmicutes | KK220717.1 | 3 | EZV78174.1 | 15422 |
| 919 | GCA_000637455.1 | 1413330 | 1280 | Staphylococcus aureus 2011-60-2256-5 | 2011-60-2256-5 | Firmicutes | KK220763.1 | 3 | EZV80246.1 | 15425 |
| 920 | GCA_000637475.1 | 1413521 | 1280 | Staphylococcus aureus 2011-60-2275-1 | 2011-60-2275-1 | Firmicutes | KK220803.1 | 3 | EZV86033.1 | 15428 |
| 921 | GCA_000637495.1 | 1413334 | 1280 | Staphylococcus aureus 22825 | 22825 | Firmicutes | KK220854.1 | 3 | EZV85085.1 | 15431 |
| 922 | GCA_000637515.1 | 1413336 | 1280 | Staphylococcus aureus 22835 | 22835 | Firmicutes | KK220886.1 | 3 | EZV88945.1 | 15434 |
| 923 | GCA_000637535.1 | 1413337 | 1280 | Staphylococcus aureus 22837 | 22837 | Firmicutes | KK220922.1 | 3 | EZV92667.1 | 15437 |
| 924 | GCA_000637555.1 | 1413338 | 1280 | Staphylococcus aureus 22838 | 22838 | Firmicutes | KK220954.1 | 3 | EZV94448.1 | 15440 |
| 925 | GCA_000637575.1 | 1413339 | 1280 | Staphylococcus aureus 22841 | 22841 | Firmicutes | KK220987.1 | 3 | EZV97126.1 | 15443 |
| 926 | GCA_000637595.1 | 1413340 | 1280 | Staphylococcus aureus 22843 | 22843 | Firmicutes | KK221024.1 | 3 | EZV98957.1 | 15446 |
| 927 | GCA_000637615.1 | 1413341 | 1280 | Staphylococcus aureus 22846 | 22846 | Firmicutes | KK221058.1 | 3 | EZW01628.1 | 15449 |
| 928 | GCA_000637635.1 | 1413438 | 1280 | Staphylococcus aureus 22P79 | 22P79 | Firmicutes | KK221090.1 | 3 | EZW04546.1 | 15452 |
| 929 | GCA_000637655.1 | 1413342 | 1280 | Staphylococcus aureus 23237 | 23237 | Firmicutes | KK221128.1 | 3 | EZW06685.1 | 15455 |
| 930 | GCA_000637675.1 | 1413414 | 1280 | Staphylococcus aureus 2393-15 | 2393-15 | Firmicutes | KK221165.1 | 3 | EZW08828.1 | 15458 |
| 931 | GCA_000637695.1 | 1413415 | 1280 | Staphylococcus aureus 2393-19 | 2393-19 | Firmicutes | KK221201.1 | 3 | EZW10960.1 | 15461 |
| 932 | GCA_000637715.1 | 1413545 | 1280 | Staphylococcus aureus 25(2889) | 25(2889) | Firmicutes | KK221232.1 | 3 | EZW14699.1 | 15464 |
| 933 | GCA_000637735.1 | 1413406 | 1280 | Staphylococcus aureus 27999-3 | 27999-3 | Firmicutes | KK221262.1 | 3 | EZW17376.1 | 15467 |
| 934 | GCA_000637775.1 | 1413589 | 1280 | Staphylococcus aureus 36P1 | 36P1 | Firmicutes | KK221358.1 | 3 | EZW21157.1 | 15470 |
| 935 | GCA_000637795.1 | 1413590 | 1280 | Staphylococcus aureus 36P5 | 36P5 | Firmicutes | KK221386.1 | 3 | EZW24847.1 | 15473 |
| 936 | GCA_000637815.1 | 1413559 | 1280 | Staphylococcus aureus 37(18S2S5-05) | 37(18S2S5-05) | Firmicutes | KK221416.1 | 3 | EZW30609.1 | 15476 |
| 937 | GCA_000637835.1 | 1413592 | 1280 | Staphylococcus aureus 40P10 | 40P10 | Firmicutes | KK221466.1 | 3 | EZW29484.1 | 15479 |
| 938 | GCA_000637855.1 | 1413591 | 1280 | Staphylococcus aureus 40P5 1_1 | 40P5 1_1 | Firmicutes | KK221506.1 | 3 | EZW34093.1 | 15482 |
| 939 | GCA_000637875.1 | 1413593 | 1280 | Staphylococcus aureus 43P3 | 43P3 | Firmicutes | KK221549.1 | 3 | EZW34360.1 | 15485 |
| 940 | GCA_000637895.1 | 1413594 | 1280 | Staphylococcus aureus 43P8 | 43P8 | Firmicutes | KK221571.1 | 3 | EZW38692.1 | 15488 |
| 941 | GCA_000637915.1 | 1413546 | 1280 | Staphylococcus aureus 44(2608) | 44(2608) | Firmicutes | KK221597.1 | 3 | EZW42269.1 | 15491 |
| 942 | GCA_000637935.1 | 1413595 | 1280 | Staphylococcus aureus 47P5 | 47P5 | Firmicutes | KK221644.1 | 3 | EZW41573.1 | 15494 |
| 943 | GCA_000637955.1 | 1413596 | 1280 | Staphylococcus aureus 47P9 | 47P9 | Firmicutes | KK221684.1 | 3 | EZW45924.1 | 15497 |
| 944 | GCA_000637975.1 | 1413384 | 1280 | Staphylococcus aureus 5670-1 | 5670-1 | Firmicutes | KK221721.1 | 3 | EZW48566.1 | 15500 |
| 945 | GCA_000637995.1 | 1413410 | 1280 | Staphylococcus aureus 56824-10 | 56824-10 | Firmicutes | KK221759.1 | 3 | EZW51956.1 | 15503 |
| 946 | GCA_000638015.1 | 1413411 | 1280 | Staphylococcus aureus 56824-15 | 56824-15 | Firmicutes | KK221811.1 | 3 | EZW52897.1 | 15506 |
| 947 | GCA_000638035.1 | 1413407 | 1280 | Staphylococcus aureus 56824-2 | 56824-2 | Firmicutes | KK221853.1 | 3 | EZW54604.1 | 15509 |
| 948 | GCA_000638055.1 | 1413412 | 1280 | Staphylococcus aureus 56824-21 | 56824-21 | Firmicutes | KK221878.1 | 3 | EZW61228.1 | 15512 |
| 949 | GCA_000638075.1 | 1413408 | 1280 | Staphylococcus aureus 56824-5 | 56824-5 | Firmicutes | KK221926.1 | 3 | EZW59349.1 | 15515 |
| 950 | GCA_000638095.1 | 1413409 | 1280 | Staphylococcus aureus 56824-7 | 56824-7 | Firmicutes | KK221958.1 | 3 | EZW64575.1 | 15518 |
| 951 | GCA_000638135.1 | 1413385 | 1280 | Staphylococcus aureus 6665-5 | 6665-5 | Firmicutes | KK222053.1 | 3 | EZW67819.1 | 15521 |
| 952 | GCA_000638155.1 | 1413395 | 1280 | Staphylococcus aureus 75495-3 | 75495-3 | Firmicutes | KK222088.1 | 3 | EZW72066.1 | 15524 |
| 953 | GCA_000638175.1 | 1413396 | 1280 | Staphylococcus aureus 76669-6 | 76669-6 | Firmicutes | KK222133.1 | 3 | EZW73745.1 | 15527 |
| 954 | GCA_000638215.1 | 1413324 | 1280 | Staphylococcus aureus 81070 | 81070 | Firmicutes | KK222240.1 | 3 | EZW78367.1 | 15530 |
| 955 | GCA_000638235.1 | 1413397 | 1280 | Staphylococcus aureus 84069-2 | 84069-2 | Firmicutes | KK222292.1 | 3 | EZW78068.1 | 15533 |
| 956 | GCA_000638255.1 | 1413398 | 1280 | Staphylococcus aureus 86770-7 | 86770-7 | Firmicutes | KK222314.1 | 3 | EZW85425.1 | 15536 |
| 957 | GCA_000638275.1 | 1413389 | 1280 | Staphylococcus aureus 87807-1 | 87807-1 | Firmicutes | KK222349.1 | 3 | EZW86269.1 | 15539 |
| 958 | GCA_000638295.1 | 1413390 | 1280 | Staphylococcus aureus 87807-11 | 87807-11 | Firmicutes | KK222381.1 | 3 | EZW90701.1 | 15542 |
| 959 | GCA_000638315.1 | 1413392 | 1280 | Staphylococcus aureus 87807-12 | 87807-12 | Firmicutes | KK222425.1 | 3 | EZW93348.1 | 15545 |
| 960 | GCA_000638335.1 | 1413393 | 1280 | Staphylococcus aureus 87807-16 | 87807-16 | Firmicutes | KK222467.1 | 3 | EZW94212.1 | 15548 |
| 961 | GCA_000638355.1 | 1413391 | 1280 | Staphylococcus aureus 87807-9 | 87807-9 | Firmicutes | KK222503.1 | 3 | EZW97015.1 | 15551 |
| 962 | GCA_000638375.1 | 1413399 | 1280 | Staphylococcus aureus 88088-1 | 88088-1 | Firmicutes | KK222532.1 | 3 | EZX00539.1 | 15554 |
| 963 | GCA_000638395.1 | 1413400 | 1280 | Staphylococcus aureus 88088-2 | 88088-2 | Firmicutes | KK222577.1 | 3 | EZX02277.1 | 15557 |
| 964 | GCA_000638415.1 | 1413586 | 1280 | Staphylococcus aureus 9P9 | 9P9 | Firmicutes | KK222619.1 | 3 | EZX04400.1 | 15560 |
| 965 | GCA_000638435.1 | 1413383 | 1280 | Staphylococcus aureus BG407 | BG407 | Firmicutes | KK222651.1 | 3 | EZX08872.1 | 15563 |
| 966 | GCA_000638475.1 | 1413509 | 1280 | Staphylococcus aureus C0637 | C0637 | Firmicutes | KK222726.1 | 3 | EZX10608.1 | 15566 |
| 967 | GCA_000638515.1 | 1413466 | 1280 | Staphylococcus aureus C1655 | C1655 | Firmicutes | KK222789.1 | 3 | EZX16970.1 | 15569 |
| 968 | GCA_000638535.1 | 1413470 | 1280 | Staphylococcus aureus C1673 | C1673 | Firmicutes | KK222826.1 | 3 | EZX19510.1 | 15572 |
| 969 | GCA_000638555.1 | 1413461 | 1280 | Staphylococcus aureus C1842 | C1842 | Firmicutes | KK222863.1 | 3 | EZX23730.1 | 15575 |
| 970 | GCA_000638575.1 | 1413458 | 1280 | Staphylococcus aureus C1891 | C1891 | Firmicutes | KK222914.1 | 3 | EZX26911.1 | 15578 |
| 971 | GCA_000638595.1 | 1413459 | 1280 | Staphylococcus aureus C1894 | C1894 | Firmicutes | KK222954.1 | 3 | EZX30320.1 | 15581 |
| 972 | GCA_000638615.1 | 1413481 | 1280 | Staphylococcus aureus C2549 | C2549 | Firmicutes | KK222984.1 | 3 | EZX30781.1 | 15584 |
| 973 | GCA_000638635.1 | 1413482 | 1280 | Staphylococcus aureus C2679 | C2679 | Firmicutes | KK223018.1 | 3 | EZX33812.1 | 15587 |
| 974 | GCA_000638655.1 | 1413467 | 1280 | Staphylococcus aureus C2706 | C2706 | Firmicutes | KK223042.1 | 3 | EZX38408.1 | 15590 |
| 975 | GCA_000638675.1 | 1413474 | 1280 | Staphylococcus aureus C2928 | C2928 | Firmicutes | KK223082.1 | 3 | EZX39864.1 | 15593 |
| 976 | GCA_000638695.1 | 1413472 | 1280 | Staphylococcus aureus C2930 | C2930 | Firmicutes | KK223136.1 | 3 | EZX38524.1 | 15596 |
| 977 | GCA_000638715.1 | 1413475 | 1280 | Staphylococcus aureus C2942 | C2942 | Firmicutes | KK223163.1 | 3 | EZX45515.1 | 15599 |
| 978 | GCA_000638735.1 | 1413454 | 1280 | Staphylococcus aureus C3489 | C3489 | Firmicutes | KK223216.1 | 3 | EZX46731.1 | 15602 |
| 979 | GCA_000638755.1 | 1413476 | 1280 | Staphylococcus aureus C3672 | C3672 | Firmicutes | KK223267.1 | 3 | EZX48305.1 | 15605 |
| 980 | GCA_000638775.1 | 1413456 | 1280 | Staphylococcus aureus C3865 | C3865 | Firmicutes | KK223311.1 | 3 | EZX53235.1 | 15608 |
| 981 | GCA_000638795.1 | 1413483 | 1280 | Staphylococcus aureus C3965 | C3965 | Firmicutes | KK223341.1 | 3 | EZX53550.1 | 15611 |
| 982 | GCA_000638815.1 | 1413477 | 1280 | Staphylococcus aureus C4151 | C4151 | Firmicutes | KK223372.1 | 3 | EZX56516.1 | 15614 |
| 983 | GCA_000638835.1 | 1413471 | 1280 | Staphylococcus aureus C4155 | C4155 | Firmicutes | KK223399.1 | 3 | EZX60548.1 | 15617 |
| 984 | GCA_000638855.1 | 1413457 | 1280 | Staphylococcus aureus C4668 | C4668 | Firmicutes | KK223430.1 | 3 | EZX62512.1 | 15620 |
| 985 | GCA_000638875.1 | 1413455 | 1280 | Staphylococcus aureus C5086 | C5086 | Firmicutes | KK223462.1 | 3 | EZX64345.1 | 15623 |
| 986 | GCA_000638895.1 | 1413480 | 1280 | Staphylococcus aureus C5453 | C5453 | Firmicutes | KK223489.1 | 3 | EZX67972.1 | 15626 |
| 987 | GCA_000638915.1 | 1413549 | 1280 | Staphylococcus aureus Chi-10 | Chi-10 | Firmicutes | KK223517.1 | 3 | EZX70983.1 | 15629 |
| 988 | GCA_000638935.1 | 1413548 | 1280 | Staphylococcus aureus Chi-4 | Chi-4 | Firmicutes | KK223557.1 | 3 | EZX70899.1 | 15632 |
| 989 | GCA_000638955.1 | 1413550 | 1280 | Staphylococcus aureus Chi-8 | Chi-8 | Firmicutes | KK223582.1 | 3 | EZX78545.1 | 15635 |
| 990 | GCA_000638975.1 | 1413577 | 1280 | Staphylococcus aureus DICM09/ | DICM09/ | Firmicutes | KK223626.1 | 3 | EZX83217.1 | 15638 |
| | | | | 00997-9HST2 | 00997-9HST2 | | | | | |
| 991 | GCA_000638995.1 | 1413580 | 1280 | Staphylococcus aureus DICM09/ | DICM09/ | Firmicutes | KK223686.1 | 3 | EZX78168.1 | 15641 |
| | | | | 01587-13HST | 01587-13HST | | | | | |
| 992 | GCA_000639015.1 | 1413439 | 1280 | Staphylococcus aureus FP_N239 | FP_N239 | Firmicutes | KK223720.1 | 3 | EZX83512.1 | 15644 |
| 993 | GCA_000639035.1 | 1413440 | 1280 | Staphylococcus aureus FP_N5203 OX | FP_N5203 OX | Firmicutes | KK223754.1 | 3 | EZX85564.1 | 15647 |
| 994 | GCA_000639055.1 | 1413441 | 1280 | Staphylococcus aureus FP_N5208 OX | FP_N5208 OX | Firmicutes | KK223787.1 | 3 | EZX88439.1 | 15650 |
| 995 | GCA_000639075.1 | 1413567 | 1280 | Staphylococcus aureus GD2010-052 | GD2010-052 | Firmicutes | KK223829.1 | 3 | EZX90794.1 | 15653 |
| 996 | GCA_000639095.1 | 1413568 | 1280 | Staphylococcus aureus GD2010-061 | GD2010-061 | Firmicutes | KK223867.1 | 3 | EZX93307.1 | 15656 |
| 997 | GCA_000639115.1 | 1413526 | 1280 | Staphylococcus aureus GD2010-090 | GD2010-090 | Firmicutes | KK223898.1 | 3 | EZX97302.1 | 15659 |
| 998 | GCA_000639135.1 | 1413569 | 1280 | Staphylococcus aureus GD2010-102 | GD2010-102 | Firmicutes | KK223946.1 | 3 | EZX98401.1 | 15662 |
| 999 | GCA_000639155.1 | 1413528 | 1280 | Staphylococcus aureus GD2010-107 | GD2010-107 | Firmicutes | KK223977.1 | 3 | EZY04106.1 | 15665 |
| 1000 | GCA_000639175.1 | 1413527 | 1280 | Staphylococcus aureus GD2010-110 | GD2010-110 | Firmicutes | KK224019.1 | 3 | EZY04548.1 | 15668 |
| 1001 | GCA_000639195.1 | 1413530 | 1280 | Staphylococcus aureus GD2010-112 | GD2010-112 | Firmicutes | KK224053.1 | 3 | EZY06602.1 | 15671 |
| 1002 | GCA_000639215.1 | 1413529 | 1280 | Staphylococcus aureus GD2010-115 | GD2010-115 | Firmicutes | KK224086.1 | 3 | EZY11332.1 | 15674 |
| 1003 | GCA_000639235.1 | 1413531 | 1280 | Staphylococcus aureus GD2010-131 | GD2010-131 | Firmicutes | KK224135.1 | 3 | EZY13792.1 | 15677 |
| 1004 | GCA_000639255.1 | 1413532 | 1280 | Staphylococcus aureus GD2010-147 | GD2010-147 | Firmicutes | KK224189.1 | 3 | EZY14132.1 | 15680 |
| 1005 | GCA_000639275.1 | 1413533 | 1280 | Staphylococcus aureus GD2010-155 | GD2010-155 | Firmicutes | KK224222.1 | 3 | EZY14216.1 | 15683 |
| 1006 | GCA_000639295.1 | 1413534 | 1280 | Staphylococcus aureus GD2010-158 | GD2010-158 | Firmicutes | KK224237.1 | 3 | EZY21814.1 | 15686 |
| 1007 | GCA_000639315.1 | 1413537 | 1280 | Staphylococcus aureus GD2010-168 | GD2010-168 | Firmicutes | KK224279.1 | 3 | EZY21674.1 | 15689 |
| 1008 | GCA_000639335.1 | 1413525 | 1280 | Staphylococcus aureus GD2010-177 | GD2010-177 | Firmicutes | KK224318.1 | 3 | EZY24519.1 | 15692 |
| 1009 | GCA_000639355.1 | 1413323 | 1280 | Staphylococcus aureus M118-B | M118-B | Firmicutes | KK224362.1 | 3 | EZY26573.1 | 15695 |
| 1010 | GCA_000639375.1 | 1413442 | 1280 | Staphylococcus aureus M267-1 | M267-1 | Firmicutes | KK224385.1 | 3 | EZY29473.1 | 15698 |
| 1011 | GCA_000639395.1 | 1413443 | 1280 | Staphylococcus aureus M357 | M357 | Firmicutes | KK224412.1 | 3 | EZY31974.1 | 15701 |
| 1012 | GCA_000639415.1 | 1413444 | 1280 | Staphylococcus aureus M457-1 | M457-1 | Firmicutes | KK224434.1 | 3 | EZY36828.1 | 15704 |
| 1013 | GCA_000639435.1 | 1413445 | 1280 | Staphylococcus aureus M520-1 | M520-1 | Firmicutes | KK224471.1 | 3 | EZY36641.1 | 15707 |
| 1014 | GCA_000639455.1 | 1413495 | 1280 | Staphylococcus aureus MRSA-118 | MRSA-118 | Firmicutes | KK224503.1 | 3 | EZY38136.1 | 15710 |
| 1015 | GCA_000639475.1 | 1413497 | 1280 | Staphylococcus aureus MRSA-136 | MRSA-136 | Firmicutes | KK224533.1 | 3 | EZY43514.1 | 15713 |
| 1016 | GCA_000639495.1 | 1413492 | 1280 | Staphylococcus aureus MSSA-93 | MSSA-93 | Firmicutes | KK224580.1 | 3 | EZY44867.1 | 15716 |
| 1017 | GCA_000639515.1 | 1413498 | 1280 | Staphylococcus aureus N0426 | N0426 | Firmicutes | KK224621.1 | 3 | EZY46959.1 | 15719 |
| 1018 | GCA_000639535.1 | 1413499 | 1280 | Staphylococcus aureus N1343 | N1343 | Firmicutes | KK224655.1 | 3 | EZY51217.1 | 15722 |
| 1019 | GCA_000639555.1 | 1413512 | 1280 | Staphylococcus aureus P0218 | P0218 | Firmicutes | KK224694.1 | 3 | EZY52189.1 | 15725 |
| 1020 | GCA_000639575.1 | 1413489 | 1280 | Staphylococcus aureus PA11 | PA11 | Firmicutes | KK224731.1 | 3 | EZY54364.1 | 15728 |
| 1021 | GCA_000639595.1 | 1413490 | 1280 | Staphylococcus aureus PA57 | PA57 | Firmicutes | KK224756.1 | 3 | EZY58964.1 | 15731 |
| 1022 | GCA_000639635.1 | 1413501 | 1280 | Staphylococcus aureus R0353 | R0353 | Firmicutes | KK224861.1 | 3 | EZY61861.1 | 15734 |
| 1023 | GCA_000639655.1 | 1413502 | 1280 | Staphylococcus aureus R0357 | R0357 | Firmicutes | KK224908.1 | 3 | EZY66174.1 | 15737 |
| 1024 | GCA_000639675.1 | 1413503 | 1280 | Staphylococcus aureus R0487 | R0487 | Firmicutes | KK224950.1 | 3 | EZY70311.1 | 15740 |
| 1025 | GCA_000639695.1 | 1413504 | 1280 | Staphylococcus aureus R0545 | R0545 | Firmicutes | KK225002.1 | 3 | EZY70617.1 | 15743 |
| 1026 | GCA_000639715.1 | 1413505 | 1280 | Staphylococcus aureus R0611 | R0611 | Firmicutes | KK225056.1 | 3 | EZY75196.1 | 15746 |
| 1027 | GCA_000639735.1 | 1413506 | 1280 | Staphylococcus aureus R0615 | R0615 | Firmicutes | KK225109.1 | 3 | EZY78995.1 | 15749 |
| 1028 | GCA_000639755.1 | 1413538 | 1280 | Staphylococcus aureus Rd.3 | Rd.3 | Firmicutes | KK225169.1 | 3 | EZY77812.1 | 15752 |
| 1029 | GCA_000639775.1 | 1413540 | 1280 | Staphylococcus aureus Rd.40 | Rd.40 | Firmicutes | KK225209.1 | 3 | EZY80413.1 | 15755 |
| 1030 | GCA_000639795.1 | 1413541 | 1280 | Staphylococcus aureus Rd.51 | Rd.51 | Firmicutes | KK225236.1 | 3 | EZY85273.1 | 15758 |
| 1031 | GCA_000639815.1 | 1413563 | 1280 | Staphylococcus aureus Rd.545 | Rd.545 | Firmicutes | KK225257.1 | 3 | EZY90749.1 | 15761 |
| 1032 | GCA_000639835.1 | 1413542 | 1280 | Staphylococcus aureus Rd.60 | Rd.60 | Firmicutes | KK225318.1 | 3 | EZY86733.1 | 15764 |
| 1033 | GCA_000639855.1 | 1413564 | 1280 | Staphylococcus aureus Rd.614 | Rd.614 | Firmicutes | KK225337.1 | 3 | EZY93710.1 | 15767 |
| 1034 | GCA_000639875.1 | 1413539 | 1280 | Staphylococcus aureus Rd.9 | Rd.9 | Firmicutes | KK225372.1 | 3 | EZY96337.1 | 15770 |
| 1035 | GCA_000639895.1 | 1413484 | 1280 | Staphylococcus aureus S56_POEL | S56_POEL | Firmicutes | KK225426.1 | 3 | EZY99137.1 | 15773 |
| 1036 | GCA_000639915.1 | 1413485 | 1280 | Staphylococcus aureus S62_POEL | S62_POEL | Firmicutes | KK225491.1 | 3 | EZZ00577.1 | 15776 |
| 1037 | GCA_000639935.1 | 1413488 | 1280 | Staphylococcus aureus S69_POEL | S69_POEL | Firmicutes | KK225547.1 | 3 | EZZ04415.1 | 15779 |
| 1038 | GCA_000639955.1 | 1413446 | 1280 | Staphylococcus aureus SARM C4155 | SARM C4155 | Firmicutes | KK225609.1 | 3 | EZZ04097.1 | 15782 |
| 1039 | GCA_000639975.1 | 1413447 | 1280 | Staphylococcus aureus SARM C5621 | SARM C5621 | Firmicutes | KK225630.1 | 3 | EZZ07028.1 | 15785 |
| 1040 | GCA_000639995.1 | 1413382 | 1280 | Staphylococcus aureus Sau 194 | Sau 194 | Firmicutes | KK225664.1 | 3 | EZZ11502.1 | 15788 |
| 1041 | GCA_000640015.1 | 1413374 | 1280 | Staphylococcus aureus Sau 44 | Sau 44 | Firmicutes | KK225696.1 | 3 | EZZ12223.1 | 15791 |
| 1042 | GCA_000640035.1 | 1413375 | 1280 | Staphylococcus aureus Sau 46 | Sau 46 | Firmicutes | KK225732.1 | 3 | EZZ13376.1 | 15794 |
| 1043 | GCA_000640055.1 | 1413376 | 1280 | Staphylococcus aureus Sau 47 | Sau 47 | Firmicutes | KK225762.1 | 3 | EZZ20017.1 | 15797 |
| 1044 | GCA_000640075.1 | 1413377 | 1280 | Staphylococcus aureus Sau 74 | Sau 74 | Firmicutes | KK225802.1 | 3 | EZZ20493.1 | 15800 |
| 1045 | GCA_000640095.1 | 1413378 | 1280 | Staphylococcus aureus Sau 93 | Sau 93 | Firmicutes | KK225836.1 | 3 | EZZ22879.1 | 15803 |
| 1046 | GCA_000640115.1 | 1413379 | 1280 | Staphylococcus aureus Sau 95 | Sau 95 | Firmicutes | KK225869.1 | 3 | EZZ27504.1 | 15806 |
| 1047 | GCA_000640135.1 | 1413380 | 1280 | Staphylococcus aureus Sau 98 | Sau 98 | Firmicutes | KK225900.1 | 3 | EZZ27419.1 | 15809 |
| 1048 | GCA_000640175.1 | 1413555 | 1280 | Staphylococcus aureus Tur-12 | Tur-12 | Firmicutes | KK225980.1 | 3 | EZZ33553.1 | 15812 |
| 1049 | GCA_000640195.1 | 1413556 | 1280 | Staphylococcus aureus Tur-15 | Tur-15 | Firmicutes | KK226022.1 | 3 | EZZ35199.1 | 15815 |
| 1050 | GCA_000640215.1 | 1413373 | 1280 | Staphylococcus aureus Tur-16 | Tur-16 | Firmicutes | KK226052.1 | 3 | EZZ38883.1 | 15818 |
| 1051 | GCA_000640235.1 | 1413551 | 1280 | Staphylococcus aureus Tur-20 | Tur-20 | Firmicutes | KK226092.1 | 3 | EZZ42314.1 | 15821 |
| 1052 | GCA_000640255.1 | 1413552 | 1280 | Staphylococcus aureus Tur-22 | Tur-22 | Firmicutes | KK226129.1 | 3 | EZZ43690.1 | 15824 |
| 1053 | GCA_000640275.1 | 1413554 | 1280 | Staphylococcus aureus Tur-6 | Tur-6 | Firmicutes | KK226169.1 | 3 | EZZ46493.1 | 15827 |
| 1054 | GCA_000640295.1 | 1413331 | 1280 | Staphylococcus aureus UB 08-116 | UB 08-116 | Firmicutes | KK226203.1 | 3 | EZZ50763.1 | 15830 |
| 1055 | GCA_000640315.1 | 1413332 | 1280 | Staphylococcus aureus UB 08-172 | UB 08-172 | Firmicutes | KK226235.1 | 3 | EZZ51037.1 | 15833 |
| 1056 | GCA_000640335.1 | 1413602 | 1280 | Staphylococcus aureus USA_1 | USA_1 | Firmicutes | KK226264.1 | 3 | EZZ53438.1 | 15836 |
| 1057 | GCA_000640355.1 | 1413607 | 1280 | Staphylococcus aureus USA_11 | USA_11 | Firmicutes | KK226275.1 | 3 | EZZ62216.1 | 15839 |
| 1058 | GCA_000640375.1 | 1413608 | 1280 | Staphylococcus aureus USA_12 | USA_12 | Firmicutes | KK226308.1 | 3 | EZZ58809.1 | 15842 |
| 1059 | GCA_000640395.1 | 1413609 | 1280 | Staphylococcus aureus USA_15 | USA_15 | Firmicutes | KK226326.1 | 3 | EZZ63777.1 | 15845 |
| 1060 | GCA_000640415.1 | 1413605 | 1280 | Staphylococcus aureus USA_8 | USA_8 | Firmicutes | KK226363.1 | 3 | EZZ66976.1 | 15848 |
| 1061 | GCA_000640435.1 | 1413606 | 1280 | Staphylococcus aureus USA_9 | USA_9 | Firmicutes | KK226405.1 | 3 | EZZ65105.1 | 15851 |
| 1062 | GCA_000640455.1 | 1413604 | 1280 | Staphylococcus aureus USA7 | USA7 | Firmicutes | KK226422.1 | 3 | EZZ70714.1 | 15854 |
| 1063 | GCA_000640475.1 | 1413448 | 1280 | Staphylococcus aureus VE07/03048STA | VE07/03048STA | Firmicutes | KK226453.1 | 3 | EZZ73603.1 | 15857 |
| 1064 | GCA_000640495.1 | 1413449 | 1280 | Staphylococcus aureus VE08/02126ST1 | VE08/02126ST1 | Firmicutes | KK226495.1 | 3 | EZZ75114.1 | 15860 |
| 1065 | GCA_000640515.1 | 1413451 | 1280 | Staphylococcus aureus VE08/02244ST1 | VE08/02244ST1 | Firmicutes | KK226529.1 | 3 | EZZ75511.1 | 15863 |
| 1066 | GCA_000640535.1 | 1422391 | 1280 | Staphylococcus aureus VET0051R | VET0051R | Firmicutes | KK226556.1 | 3 | EZZ82800.1 | 15866 |
| 1067 | GCA_000640555.1 | 1422392 | 1280 | Staphylococcus aureus VET0052R | VET0052R | Firmicutes | KK226598.1 | 3 | EZZ83623.1 | 15869 |
| 1068 | GCA_000640575.1 | 1422393 | 1280 | Staphylococcus aureus VET0053R | VET0053R | Firmicutes | KK226639.1 | 3 | EZZ82655.1 | 15872 |
| 1069 | GCA_000640595.1 | 1422394 | 1280 | Staphylococcus aureus VET0054R | VET0054R | Firmicutes | KK226674.1 | 3 | EZZ89015.1 | 15875 |
| 1070 | GCA_000640615.1 | 1422395 | 1280 | Staphylococcus aureus VET0055R | VET0055R | Firmicutes | KK226711.1 | 3 | EZZ92962.1 | 15878 |
| 1071 | GCA_000640635.1 | 1422396 | 1280 | Staphylococcus aureus VET0057R | VET0057R | Firmicutes | KK226773.1 | 3 | EZZ92684.1 | 15881 |
| 1072 | GCA_000640655.1 | 1422398 | 1280 | Staphylococcus aureus VET0059R | VET0059R | Firmicutes | KK226834.1 | 3 | EZZ95684.1 | 15884 |
| 1073 | GCA_000640675.1 | 1422399 | 1280 | Staphylococcus aureus VET0060R | VET0060R | Firmicutes | KK226872.1 | 3 | KAA05377.1 | 15887 |
| 1074 | GCA_000640695.1 | 1422400 | 1280 | Staphylococcus aureus VET0061R | VET0061R | Firmicutes | KK226919.1 | 3 | KAA05544.1 | 15890 |
| 1075 | GCA_000640715.1 | 1422401 | 1280 | Staphylococcus aureus VET0062R | VET0062R | Firmicutes | KK226958.1 | 3 | KAA05953.1 | 15893 |
| 1076 | GCA_000640735.1 | 1422402 | 1280 | Staphylococcus aureus VET0063R | VET0063R | Firmicutes | KK226999.1 | 3 | KAA13000.1 | 15896 |
| 1077 | GCA_000640755.1 | 1422403 | 1280 | Staphylococcus aureus VET0065R | VET0065R | Firmicutes | KK227042.1 | 3 | KAA12393.1 | 15899 |
| 1078 | GCA_000640775.1 | 1422404 | 1280 | Staphylococcus aureus VET0066R | VET0066R | Firmicutes | KK227076.1 | 3 | KAA13619.1 | 15902 |
| 1079 | GCA_000640795.1 | 1422405 | 1280 | Staphylococcus aureus VET0067R | VET0067R | Firmicutes | KK227106.1 | 3 | KAA22643.1 | 15905 |
| 1080 | GCA_000640815.1 | 1422406 | 1280 | Staphylococcus aureus VET0069R | VET0069R | Firmicutes | KK227159.1 | 3 | KAA21033.1 | 15908 |
| 1081 | GCA_000640835.1 | 1422407 | 1280 | Staphylococcus aureus VET0070R | VET0070R | Firmicutes | KK227206.1 | 3 | KAA21282.1 | 15911 |
| 1082 | GCA_000640855.1 | 1422408 | 1280 | Staphylococcus aureus VET0072R | VET0072R | Firmicutes | KK227240.1 | 3 | KAA28470.1 | 15914 |
| 1083 | GCA_000640875.1 | 1422409 | 1280 | Staphylococcus aureus VET0073R | VET0073R | Firmicutes | KK227288.1 | 3 | KAA29978.1 | 15917 |
| 1084 | GCA_000640895.1 | 1422410 | 1280 | Staphylococcus aureus VET0075R | VET0075R | Firmicutes | KK227338.1 | 3 | KAA28571.1 | 15920 |
| 1085 | GCA_000640915.1 | 1422411 | 1280 | Staphylococcus aureus VET0076R | VET0076R | Firmicutes | KK227375.1 | 3 | KAA35386.1 | 15923 |
| 1086 | GCA_000640935.1 | 1422412 | 1280 | Staphylococcus aureus VET0077R | VET0077R | Firmicutes | KK227403.1 | 3 | KAA38915.1 | 15926 |
| 1087 | GCA_000640955.1 | 1422414 | 1280 | Staphylococcus aureus VET0080R | VET0080R | Firmicutes | KK227448.1 | 3 | KAA39462.1 | 15929 |
| 1088 | GCA_000640975.1 | 1422416 | 1280 | Staphylococcus aureus VET0083R | VET0083R | Firmicutes | KK227491.1 | 3 | KAA42954.1 | 15932 |
| 1089 | GCA_000640995.1 | 1422417 | 1280 | Staphylococcus aureus VET0084R | VET0084R | Firmicutes | KK227523.1 | 3 | KAA45225.1 | 15935 |
| 1090 | GCA_000641015.1 | 1422418 | 1280 | Staphylococcus aureus VET0085R | VET0085R | Firmicutes | KK227565.1 | 3 | KAA46801.1 | 15938 |
| 1091 | GCA_000641035.1 | 1422419 | 1280 | Staphylococcus aureus VET0086R | VET0086R | Firmicutes | KK227605.1 | 3 | KAA49913.1 | 15941 |
| 1092 | GCA_000641055.1 | 1422420 | 1280 | Staphylococcus aureus VET0087R | VET0087R | Firmicutes | KK227638.1 | 3 | KAA51796.1 | 15944 |
| 1093 | GCA_000641075.1 | 1422421 | 1280 | Staphylococcus aureus VET0088R | VET0088R | Firmicutes | KK227658.1 | 3 | KAA59416.1 | 15947 |
| 1094 | GCA_000641095.1 | 1422422 | 1280 | Staphylococcus aureus VET0089R | VET0089R | Firmicutes | KK227710.1 | 3 | KAA58407.1 | 15950 |
| 1095 | GCA_000641115.1 | 1422423 | 1280 | Staphylococcus aureus VET0090R | VET0090R | Firmicutes | KK227736.1 | 3 | KAA61962.1 | 15953 |
| 1096 | GCA_000641135.1 | 1422424 | 1280 | Staphylococcus aureus VET0091R | VET0091R | Firmicutes | KK227783.1 | 3 | KAA63552.1 | 15956 |
| 1097 | GCA_000641155.1 | 1422425 | 1280 | Staphylococcus aureus VET0092R | VET0092R | Firmicutes | KK227826.1 | 3 | KAA66140.1 | 15959 |
| 1098 | GCA_000641175.1 | 1422426 | 1280 | Staphylococcus aureus VET0093R | VET0093R | Firmicutes | KK227870.1 | 3 | KAA68224.1 | 15962 |
| 1099 | GCA_000641195.1 | 1422427 | 1280 | Staphylococcus aureus VET0094R | VET0094R | Firmicutes | KK227907.1 | 3 | KAA73513.1 | 15965 |
| 1100 | GCA_000641215.1 | 1422428 | 1280 | Staphylococcus aureus VET0095R | VET0095R | Firmicutes | KK227954.1 | 3 | KAA74080.1 | 15968 |
| 1101 | GCA_000641235.1 | 1422429 | 1280 | Staphylococcus aureus VET0097R | VET0097R | Firmicutes | KK227993.1 | 3 | KAA76198.1 | 15971 |
| 1102 | GCA_000641255.1 | 1422430 | 1280 | Staphylococcus aureus VET0098R | VET0098R | Firmicutes | KK228029.1 | 3 | KAA79422.1 | 15974 |
| 1103 | GCA_000641275.1 | 1422431 | 1280 | Staphylococcus aureus VET0099R | VET0099R | Firmicutes | KK228070.1 | 3 | KAA82180.1 | 15977 |
| 1104 | GCA_000641295.1 | 1422432 | 1280 | Staphylococcus aureus VET0101R | VET0101R | Firmicutes | KK228126.1 | 3 | KAA82201.1 | 15980 |
| 1105 | GCA_000641315.1 | 1422434 | 1280 | Staphylococcus aureus VET0103R | VET0103R | Firmicutes | KK228153.1 | 3 | KAA86323.1 | 15983 |
| 1106 | GCA_000641335.1 | 1422435 | 1280 | Staphylococcus aureus VET0104R | VET0104R | Firmicutes | KK228187.1 | 3 | KAA89640.1 | 15986 |
| 1107 | GCA_000641355.1 | 1422436 | 1280 | Staphylococcus aureus VET0105R | VET0105R | Firmicutes | KK228218.1 | 3 | KAA93204.1 | 15989 |
| 1108 | GCA_000641375.1 | 1422437 | 1280 | Staphylococcus aureus VET0107R | VET0107R | Firmicutes | KK228279.1 | 3 | KAA93403.1 | 15992 |
| 1109 | GCA_000641395.1 | 1422438 | 1280 | Staphylococcus aureus VET0110R | VET0110R | Firmicutes | KK228324.1 | 3 | KAA96433.1 | 15995 |
| 1110 | GCA_000641415.1 | 1422440 | 1280 | Staphylococcus aureus VET0112R | VET0112R | Firmicutes | KK228363.1 | 3 | KAB00569.1 | 15998 |
| 1111 | GCA_000641435.1 | 1422442 | 1280 | Staphylococcus aureus VET0114R | VET0114R | Firmicutes | KK228395.1 | 3 | KAB03847.1 | 16001 |
| 1112 | GCA_000641455.1 | 1422443 | 1280 | Staphylococcus aureus VET0115R | VET0115R | Firmicutes | KK228451.1 | 3 | KAB06658.1 | 16004 |
| 1113 | GCA_000641475.1 | 1422444 | 1280 | Staphylococcus aureus VET0116R | VET0116R | Firmicutes | KK228505.1 | 3 | KAB08922.1 | 16007 |
| 1114 | GCA_000641495.1 | 1422445 | 1280 | Staphylococcus aureus VET0117R | VET0117R | Firmicutes | KK228551.1 | 3 | KAB10432.1 | 16010 |
| 1115 | GCA_000641515.1 | 1422446 | 1280 | Staphylococcus aureus VET0119R | VET0119R | Firmicutes | KK228588.1 | 3 | KAB11616.1 | 16013 |
| 1116 | GCA_000641535.1 | 1422448 | 1280 | Staphylococcus aureus VET0123R | VET0123R | Firmicutes | KK228618.1 | 3 | KAB16350.1 | 16016 |
| 1117 | GCA_000641555.1 | 1422449 | 1280 | Staphylococcus aureus VET0124R | VET0124R | Firmicutes | KK228659.1 | 3 | KAB18689.1 | 16019 |
| 1118 | GCA_000641575.1 | 1422451 | 1280 | Staphylococcus aureus VET0126R | VET0126R | Firmicutes | KK228701.1 | 3 | KAB19935.1 | 16022 |
| 1119 | GCA_000641595.1 | 1422452 | 1280 | Staphylococcus aureus VET0127R | VET0127R | Firmicutes | KK228748.1 | 3 | KAB24080.1 | 16025 |
| 1120 | GCA_000641615.1 | 1422453 | 1280 | Staphylococcus aureus VET0128R | VET0128R | Firmicutes | KK228781.1 | 3 | KAB29327.1 | 16028 |
| 1121 | GCA_000641635.1 | 1422454 | 1280 | Staphylococcus aureus VET0129R | VET0129R | Firmicutes | KK228823.1 | 3 | KAB29477.1 | 16031 |
| 1122 | GCA_000641655.1 | 1422455 | 1280 | Staphylococcus aureus VET0130R | VET0130R | Firmicutes | KK228852.1 | 3 | KAB32012.1 | 16034 |
| 1123 | GCA_000641675.1 | 1422456 | 1280 | Staphylococcus aureus VET0132R | VET0132R | Firmicutes | KK228890.1 | 3 | KAB33825.1 | 16037 |
| 1124 | GCA_000641695.1 | 1422457 | 1280 | Staphylococcus aureus VET0133R | VET0133R | Firmicutes | KK228932.1 | 3 | KAB37793.1 | 16040 |
| 1125 | GCA_000641715.1 | 1422458 | 1280 | Staphylococcus aureus VET0134R | VET0134R | Firmicutes | KK228988.1 | 3 | KAB38132.1 | 16043 |
| 1126 | GCA_000641735.1 | 1422460 | 1280 | Staphylococcus aureus VET0140R | VET0140R | Firmicutes | KK229019.1 | 3 | KAB42667.1 | 16046 |
| 1127 | GCA_000641755.1 | 1422462 | 1280 | Staphylococcus aureus VET0148R | VET0148R | Firmicutes | KK229051.1 | 3 | KAB46003.1 | 16049 |
| 1128 | GCA_000641775.1 | 1422464 | 1280 | Staphylococcus aureus VET0151R | VET0151R | Firmicutes | KK229081.1 | 3 | KAB45741.1 | 16052 |
| 1129 | GCA_000641795.1 | 1422465 | 1280 | Staphylococcus aureus VET0152R | VET0152R | Firmicutes | KK229110.1 | 3 | KAB51677.1 | 16055 |
| 1130 | GCA_000641815.1 | 1422467 | 1280 | Staphylococcus aureus VET0155R | VET0155R | Firmicutes | KK229172.1 | 3 | KAB53193.1 | 16058 |
| 1131 | GCA_000641835.1 | 1422468 | 1280 | Staphylococcus aureus VET0156R | VET0156R | Firmicutes | KK229219.1 | 3 | KAB55784.1 | 16061 |
| 1132 | GCA_000641855.1 | 1422472 | 1280 | Staphylococcus aureus VET0161R | VET0161R | Firmicutes | KK229266.1 | 3 | KAB58082.1 | 16064 |
| 1133 | GCA_000641875.1 | 1422473 | 1280 | Staphylococcus aureus VET0162R | VET0162R | Firmicutes | KK229299.1 | 3 | KAB59419.1 | 16067 |
| 1134 | GCA_000641895.1 | 1422474 | 1280 | Staphylococcus aureus VET0164R | VET0164R | Firmicutes | KK229332.1 | 3 | KAB62993.1 | 16070 |
| 1135 | GCA_000641915.1 | 1422475 | 1280 | Staphylococcus aureus VET0165R | VET0165R | Firmicutes | KK229377.1 | 3 | KAB66109.1 | 16073 |
| 1136 | GCA_000641935.1 | 1422476 | 1280 | Staphylococcus aureus VET0166R | VET0166R | Firmicutes | KK229442.1 | 3 | KAB66874.1 | 16076 |
| 1137 | GCA_000641955.1 | 1422477 | 1280 | Staphylococcus aureus VET0167R | VET0167R | Firmicutes | KK229493.1 | 3 | KAB70776.1 | 16079 |
| 1138 | GCA_000641975.1 | 1422478 | 1280 | Staphylococcus aureus VET0171R | VET0171R | Firmicutes | KK229539.1 | 3 | KAB73064.1 | 16082 |
| 1139 | GCA_000641995.1 | 1422479 | 1280 | Staphylococcus aureus VET0176R | VET0176R | Firmicutes | KK229579.1 | 3 | KAB75150.1 | 16085 |
| 1140 | GCA_000642015.1 | 1422480 | 1280 | Staphylococcus aureus VET0177R | VET0177R | Firmicutes | KK229612.1 | 3 | KAB78038.1 | 16088 |
| 1141 | GCA_000642035.1 | 1422481 | 1280 | Staphylococcus aureus VET0178R | VET0178R | Firmicutes | KK229644.1 | 3 | KAB81593.1 | 16091 |
| 1142 | GCA_000642055.1 | 1422482 | 1280 | Staphylococcus aureus VET0179R | VET0179R | Firmicutes | KK229686.1 | 3 | KAB84387.1 | 16094 |
| 1143 | GCA_000642075.1 | 1422483 | 1280 | Staphylococcus aureus VET0180R | VET0180R | Firmicutes | KK229729.1 | 3 | KAB84694.1 | 16097 |
| 1144 | GCA_000642095.1 | 1422488 | 1280 | Staphylococcus aureus VET0188R | VET0188R | Firmicutes | KK229754.1 | 3 | KAB91416.1 | 16100 |
| 1145 | GCA_000642115.1 | 1422489 | 1280 | Staphylococcus aureus VET0189R | VET0189R | Firmicutes | KK229800.1 | 3 | KAB92906.1 | 16103 |
| 1146 | GCA_000642135.1 | 1422490 | 1280 | Staphylococcus aureus VET0190R | VET0190R | Firmicutes | KK229842.1 | 3 | KAB93617.1 | 16106 |
| 1147 | GCA_000642155.1 | 1422492 | 1280 | Staphylococcus aureus VET0192R | VET0192R | Firmicutes | KK229894.1 | 3 | KAB98475.1 | 16109 |
| 1148 | GCA_000642175.1 | 1422493 | 1280 | Staphylococcus aureus VET0193R | VET0193R | Firmicutes | KK229956.1 | 3 | KAB98436.1 | 16112 |
| 1149 | GCA_000642195.1 | 1422494 | 1280 | Staphylococcus aureus VET0194R | VET0194R | Firmicutes | KK230007.1 | 3 | KAC00309.1 | 16115 |
| 1150 | GCA_000642215.1 | 1422495 | 1280 | Staphylococcus aureus VET0195R | VET0195R | Firmicutes | KK230044.1 | 3 | KAC04037.1 | 16118 |
| 1151 | GCA_000642235.1 | 1422498 | 1280 | Staphylococcus aureus VET0198R | VET0198R | Firmicutes | KK230081.1 | 3 | KAC08058.1 | 16121 |
| 1152 | GCA_000642255.1 | 1422500 | 1280 | Staphylococcus aureus VET0200R | VET0200R | Firmicutes | KK230124.1 | 3 | KAC10255.1 | 16124 |
| 1153 | GCA_000642275.1 | 1422508 | 1280 | Staphylococcus aureus VET0212R | VET0212R | Firmicutes | KK230171.1 | 3 | KAC11816.1 | 16127 |
| 1154 | GCA_000642295.1 | 1422509 | 1280 | Staphylococcus aureus VET0213R | VET0213R | Firmicutes | KK230206.1 | 3 | KAC14781.1 | 16130 |
| 1155 | GCA_000642315.1 | 1422510 | 1280 | Staphylococcus aureus VET0214R | VET0214R | Firmicutes | KK230245.1 | 3 | KAC17658.1 | 16133 |
| 1156 | GCA_000642335.1 | 1422511 | 1280 | Staphylococcus aureus VET0215R | VET0215R | Firmicutes | KK230294.1 | 3 | KAC20377.1 | 16136 |
| 1157 | GCA_000642355.1 | 1422512 | 1280 | Staphylococcus aureus VET0216R | VET0216R | Firmicutes | KK230357.1 | 3 | KAC21946.1 | 16139 |
| 1158 | GCA_000642375.1 | 1422513 | 1280 | Staphylococcus aureus VET0217R | VET0217R | Firmicutes | KK230406.1 | 3 | KAC25278.1 | 16142 |
| 1159 | GCA_000642395.1 | 1422514 | 1280 | Staphylococcus aureus VET0218R | VET0218R | Firmicutes | KK230455.1 | 3 | KAC29151.1 | 16145 |
| 1160 | GCA_000642415.1 | 1422516 | 1280 | Staphylococcus aureus VET0221R | VET0221R | Firmicutes | KK230491.1 | 3 | KAC33554.1 | 16148 |
| 1161 | GCA_000642435.1 | 1422517 | 1280 | Staphylococcus aureus VET0222R | VET0222R | Firmicutes | KK230550.1 | 3 | KAC34157.1 | 16151 |
| 1162 | GCA_000642455.1 | 1422518 | 1280 | Staphylococcus aureus VET0224R | VET0224R | Firmicutes | KK230611.1 | 3 | KAC33241.1 | 16154 |
| 1163 | GCA_000642475.1 | 1422519 | 1280 | Staphylococcus aureus VET0227R | VET0227R | Firmicutes | KK230635.1 | 3 | KAC37672.1 | 16157 |
| 1164 | GCA_000642495.1 | 1422520 | 1280 | Staphylococcus aureus VET0228R | VET0228R | Firmicutes | KK230680.1 | 3 | KAC40880.1 | 16160 |
| 1165 | GCA_000642515.1 | 1422522 | 1280 | Staphylococcus aureus VET0230R | VET0230R | Firmicutes | KK230709.1 | 3 | KAC45126.1 | 16163 |
| 1166 | GCA_000642535.1 | 1422525 | 1280 | Staphylococcus aureus VET0236R | VET0236R | Firmicutes | KK230757.1 | 3 | KAC44749.1 | 16166 |
| 1167 | GCA_000642555.1 | 1422526 | 1280 | Staphylococcus aureus VET0237R | VET0237R | Firmicutes | KK230781.1 | 3 | KAC51539.1 | 16169 |
| 1168 | GCA_000642575.1 | 1422527 | 1280 | Staphylococcus aureus VET0241R | VET0241R | Firmicutes | KK230828.1 | 3 | KAC50575.1 | 16172 |
| 1169 | GCA_000642595.1 | 1422528 | 1280 | Staphylococcus aureus VET0243R | VET0243R | Firmicutes | KK230863.1 | 3 | KAC52444.1 | 16175 |
| 1170 | GCA_000642615.1 | 1422530 | 1280 | Staphylococcus aureus VET0247R | VET0247R | Firmicutes | KK230896.1 | 3 | KAC57903.1 | 16178 |
| 1171 | GCA_000642635.1 | 1422531 | 1280 | Staphylococcus aureus VET0249R | VET0249R | Firmicutes | KK230930.1 | 3 | KAC59267.1 | 16181 |
| 1172 | GCA_000642655.1 | 1422533 | 1280 | Staphylococcus aureus VET0252R | VET0252R | Firmicutes | KK230956.1 | 3 | KAC64902.1 | 16184 |
| 1173 | GCA_000642675.1 | 1422534 | 1280 | Staphylococcus aureus VET0253R | VET0253R | Firmicutes | KK231012.1 | 3 | KAC65216.1 | 16187 |
| 1174 | GCA_000642695.1 | 1422535 | 1280 | Staphylococcus aureus VET0254R | VET0254R | Firmicutes | KK231053.1 | 3 | KAC66579.1 | 16190 |
| 1175 | GCA_000642715.1 | 1422536 | 1280 | Staphylococcus aureus VET0256R | VET0256R | Firmicutes | KK231074.1 | 3 | KAC76251.1 | 16193 |
| 1176 | GCA_000642735.1 | 1422537 | 1280 | Staphylococcus aureus VET0259R | VET0259R | Firmicutes | KK231155.1 | 3 | KAC70676.1 | 16196 |
| 1177 | GCA_000642755.1 | 1422538 | 1280 | Staphylococcus aureus VET0260R | VET0260R | Firmicutes | KK231188.1 | 3 | KAC73890.1 | 16199 |
| 1178 | GCA_000642775.1 | 1422539 | 1280 | Staphylococcus aureus VET0261R | VET0261R | Firmicutes | KK231221.1 | 3 | KAC78737.1 | 16202 |
| 1179 | GCA_000642795.1 | 1422540 | 1280 | Staphylococcus aureus VET0263R | VET0263R | Firmicutes | KK231265.1 | 3 | KAC82512.1 | 16205 |
| 1180 | GCA_000642815.1 | 1422541 | 1280 | Staphylococcus aureus VET0267R | VET0267R | Firmicutes | KK231317.1 | 3 | KAC80332.1 | 16208 |
| 1181 | GCA_000642835.1 | 1422542 | 1280 | Staphylococcus aureus VET0268R | VET0268R | Firmicutes | KK231348.1 | 3 | KAC86159.1 | 16211 |
| 1182 | GCA_000642855.1 | 1422543 | 1280 | Staphylococcus aureus VET0269R | VET0269R | Firmicutes | KK231386.1 | 3 | KAC89254.1 | 16214 |
| 1183 | GCA_000642875.1 | 1422544 | 1280 | Staphylococcus aureus VET0270R | VET0270R | Firmicutes | KK231435.1 | 3 | KAC89181.1 | 16217 |
| 1184 | GCA_000642895.1 | 1422545 | 1280 | Staphylococcus aureus VET0271R | VET0271R | Firmicutes | KK231470.1 | 3 | KAC93844.1 | 16220 |
| 1185 | GCA_000642915.1 | 1422546 | 1280 | Staphylococcus aureus VET0275R | VET0275R | Firmicutes | KK231503.1 | 3 | KAC96630.1 | 16223 |
| 1186 | GCA_000642935.1 | 1422547 | 1280 | Staphylococcus aureus VET0278R | VET0278R | Firmicutes | KK231552.1 | 3 | KAC97653.1 | 16226 |
| 1187 | GCA_000642955.1 | 1422548 | 1280 | Staphylococcus aureus VET0279R | VET0279R | Firmicutes | KK231605.1 | 3 | KAD00064.1 | 16229 |
| 1188 | GCA_000642975.1 | 1422549 | 1280 | Staphylococcus aureus VET0280R | VET0280R | Firmicutes | KK231633.1 | 3 | KAD04306.1 | 16232 |
| 1189 | GCA_000642995.1 | 1422550 | 1280 | Staphylococcus aureus VET0282R | VET0282R | Firmicutes | KK231663.1 | 3 | KAD08152.1 | 16235 |
| 1190 | GCA_000643015.1 | 1422551 | 1280 | Staphylococcus aureus VET0283R | VET0283R | Firmicutes | KK231710.1 | 3 | KAD07875.1 | 16238 |
| 1191 | GCA_000643035.1 | 1422552 | 1280 | Staphylococcus aureus VET0284R | VET0284R | Firmicutes | KK231759.1 | 3 | KAD11428.1 | 16241 |
| 1192 | GCA_000643055.1 | 1422553 | 1280 | Staphylococcus aureus VET0286R | VET0286R | Firmicutes | KK231797.1 | 3 | KAD14196.1 | 16244 |
| 1193 | GCA_000643075.1 | 1422554 | 1280 | Staphylococcus aureus VET0287R | VET0287R | Firmicutes | KK231839.1 | 3 | KAD16933.1 | 16247 |
| 1194 | GCA_000643095.1 | 1422555 | 1280 | Staphylococcus aureus VET0288R | VET0288R | Firmicutes | KK231877.1 | 3 | KAD20112.1 | 16250 |
| 1195 | GCA_000643115.1 | 1422556 | 1280 | Staphylococcus aureus VET0289R | VET0289R | Firmicutes | KK231905.1 | 3 | KAD22452.1 | 16253 |
| 1196 | GCA_000643135.1 | 1422557 | 1280 | Staphylococcus aureus VET0290R | VET0290R | Firmicutes | KK231934.1 | 3 | KAD23853.1 | 16256 |
| 1197 | GCA_000643155.1 | 1422558 | 1280 | Staphylococcus aureus VET0291R | VET0291R | Firmicutes | KK231957.1 | 3 | KAD27442.1 | 16259 |
| 1198 | GCA_000643175.1 | 1422559 | 1280 | Staphylococcus aureus VET0292R | VET0292R | Firmicutes | KK231982.1 | 3 | KAD29135.1 | 16262 |
| 1199 | GCA_000643195.1 | 1422561 | 1280 | Staphylococcus aureus VET0294R | VET0294R | Firmicutes | KK232006.1 | 3 | KAD33183.1 | 16265 |
| 1200 | GCA_000643215.1 | 1422562 | 1280 | Staphylococcus aureus VET0295R | VET0295R | Firmicutes | KK232044.1 | 3 | KAD35158.1 | 16268 |
| 1201 | GCA_000643235.1 | 1422563 | 1280 | Staphylococcus aureus VET0296R | VET0296R | Firmicutes | KK232071.1 | 3 | KAD41139.1 | 16271 |
| 1202 | GCA_000643255.1 | 1422564 | 1280 | Staphylococcus aureus VET0297R | VET0297R | Firmicutes | KK232123.1 | 3 | KAD37497.1 | 16274 |
| 1203 | GCA_000643275.1 | 1422566 | 1280 | Staphylococcus aureus VET0299R | VET0299R | Firmicutes | KK232151.1 | 3 | KAD41938.1 | 16277 |
| 1204 | GCA_000643295.1 | 1422567 | 1280 | Staphylococcus aureus VET0300R | VET0300R | Firmicutes | KK232180.1 | 3 | KAD44775.1 | 16280 |
| 1205 | GCA_000643315.1 | 1422569 | 1280 | Staphylococcus aureus VET0302R | VET0302R | Firmicutes | KK232221.1 | 3 | KAD49432.1 | 16283 |
| 1206 | GCA_000643335.1 | 1422570 | 1280 | Staphylococcus aureus VET0303R | VET0303R | Firmicutes | KK232282.1 | 3 | KAD48763.1 | 16286 |
| 1207 | GCA_000643355.1 | 1422571 | 1280 | Staphylococcus aureus VET0304R | VET0304R | Firmicutes | KK232321.1 | 3 | KAD53925.1 | 16289 |
| 1208 | GCA_000643375.1 | 1422572 | 1280 | Staphylococcus aureus VET0305R | VET0305R | Firmicutes | KK232371.1 | 3 | KAD57265.1 | 16292 |
| 1209 | GCA_000643395.1 | 1422574 | 1280 | Staphylococcus aureus VET0307R | VET0307R | Firmicutes | KK232424.1 | 3 | KAD59398.1 | 16295 |
| 1210 | GCA_000643415.1 | 1422575 | 1280 | Staphylococcus aureus VET0308R | VET0308R | Firmicutes | KK232479.1 | 3 | KAD59590.1 | 16298 |
| 1211 | GCA_000643435.1 | 1422576 | 1280 | Staphylococcus aureus VET0309R | VET0309R | Firmicutes | KK232506.1 | 3 | KAD64095.1 | 16301 |
| 1212 | GCA_000643455.1 | 1422577 | 1280 | Staphylococcus aureus VET0312R | VET0312R | Firmicutes | KK232547.1 | 3 | KAD64971.1 | 16304 |
| 1213 | GCA_000643475.1 | 1422578 | 1280 | Staphylococcus aureus VET0313R | VET0313R | Firmicutes | KK232579.1 | 3 | KAD71132.1 | 16307 |
| 1214 | GCA_000643495.1 | 1422579 | 1280 | Staphylococcus aureus VET0314R | VET0314R | Firmicutes | KK232626.1 | 3 | KAD76144.1 | 16310 |
| 1215 | GCA_000643515.1 | 1422580 | 1280 | Staphylococcus aureus VET0315R | VET0315R | Firmicutes | KK232671.1 | 3 | KAD70469.1 | 16313 |
| 1216 | GCA_000643535.1 | 1422581 | 1280 | Staphylococcus aureus VET0316R | VET0316R | Firmicutes | KK232698.1 | 3 | KAD78845.1 | 16316 |
| 1217 | GCA_000643555.1 | 1422582 | 1280 | Staphylococcus aureus VET0317R | VET0317R | Firmicutes | KK232759.1 | 3 | KAD77330.1 | 16319 |
| 1218 | GCA_000643575.1 | 1422583 | 1280 | Staphylococcus aureus VET0318R | VET0318R | Firmicutes | KK232802.1 | 3 | KAD80657.1 | 16322 |
| 1219 | GCA_000643595.1 | 1422585 | 1280 | Staphylococcus aureus VET0320R | VET0320R | Firmicutes | KK232846.1 | 3 | KAD85222.1 | 16325 |
| 1220 | GCA_000643615.1 | 1422586 | 1280 | Staphylococcus aureus VET0322R | VET0322R | Firmicutes | KK232899.1 | 3 | KAD85662.1 | 16328 |
| 1221 | GCA_000643635.1 | 1422587 | 1280 | Staphylococcus aureus VET0323R | VET0323R | Firmicutes | KK232949.1 | 3 | KAD90247.1 | 16331 |
| 1222 | GCA_000643655.1 | 1422588 | 1280 | Staphylococcus aureus VET0325R | VET0325R | Firmicutes | KK233004.1 | 3 | KAD93694.1 | 16334 |
| 1223 | GCA_000643675.1 | 1422589 | 1280 | Staphylococcus aureus VET0326R | VET0326R | Firmicutes | KK233060.1 | 3 | KAD94381.1 | 16337 |
| 1224 | GCA_000643695.1 | 1422590 | 1280 | Staphylococcus aureus VET0329R | VET0329R | Firmicutes | KK233107.1 | 3 | KAD99176.1 | 16340 |
| 1225 | GCA_000643715.1 | 1422591 | 1280 | Staphylococcus aureus VET0331R | VET0331R | Firmicutes | KK233161.1 | 3 | KAD98842.1 | 16343 |
| 1226 | GCA_000643735.1 | 1422593 | 1280 | Staphylococcus aureus VET0333R | VET0333R | Firmicutes | KK233217.1 | 3 | KAD99163.1 | 16346 |
| 1227 | GCA_000643755.1 | 1422594 | 1280 | Staphylococcus aureus VET0335R | VET0335R | Firmicutes | KK233273.1 | 3 | KAE03357.1 | 16349 |
| 1228 | GCA_000643775.1 | 1422595 | 1280 | Staphylococcus aureus VET0336R | VET0336R | Firmicutes | KK233293.1 | 3 | KAE06091.1 | 16352 |
| 1229 | GCA_000643795.1 | 1422596 | 1280 | Staphylococcus aureus VET0337R | VET0337R | Firmicutes | KK233320.1 | 3 | KAE10343.1 | 16355 |
| 1230 | GCA_000643815.1 | 1422597 | 1280 | Staphylococcus aureus VET0338R | VET0338R | Firmicutes | KK233365.1 | 3 | KAE11832.1 | 16358 |
| 1231 | GCA_000643835.1 | 1422598 | 1280 | Staphylococcus aureus VET0339R | VET0339R | Firmicutes | KK233392.1 | 3 | KAE16058.1 | 16361 |
| 1232 | GCA_000643855.1 | 1422599 | 1280 | Staphylococcus aureus VET0340R | VET0340R | Firmicutes | KK233439.1 | 3 | KAE17680.1 | 16364 |
| 1233 | GCA_000643875.1 | 1422600 | 1280 | Staphylococcus aureus VET0341R | VET0341R | Firmicutes | KK233484.1 | 3 | KAE21314.1 | 16367 |
| 1234 | GCA_000643895.1 | 1422603 | 1280 | Staphylococcus aureus VET0352R | VET0352R | Firmicutes | KK233526.1 | 3 | KAE22706.1 | 16370 |
| 1235 | GCA_000643915.1 | 1422605 | 1280 | Staphylococcus aureus VET0354R | VET0354R | Firmicutes | KK233565.1 | 3 | KAE26218.1 | 16373 |
| 1236 | GCA_000643935.1 | 1422606 | 1280 | Staphylococcus aureus VET0357R | VET0357R | Firmicutes | KK233602.1 | 3 | KAE28381.1 | 16376 |
| 1237 | GCA_000643955.1 | 1422608 | 1280 | Staphylococcus aureus VET0359R | VET0359R | Firmicutes | KK233637.1 | 3 | KAE30967.1 | 16379 |
| 1238 | GCA_000643975.1 | 1422609 | 1280 | Staphylococcus aureus VET0360R | VET0360R | Firmicutes | KK233668.1 | 3 | KAE34716.1 | 16382 |
| 1239 | GCA_000643995.1 | 1422611 | 1280 | Staphylococcus aureus VET0363R | VET0363R | Firmicutes | KK233714.1 | 3 | KAE36917.1 | 16385 |
| 1240 | GCA_000644015.1 | 1422613 | 1280 | Staphylococcus aureus VET0366R | VET0366R | Firmicutes | KK233762.1 | 3 | KAE38769.1 | 16388 |
| 1241 | GCA_000644035.1 | 1422614 | 1280 | Staphylococcus aureus VET0367R | VET0367R | Firmicutes | KK233806.1 | 3 | KAE42476.1 | 16391 |
| 1242 | GCA_000644055.1 | 1422616 | 1280 | Staphylococcus aureus VET0369R | VET0369R | Firmicutes | KK233856.1 | 3 | KAE45135.1 | 16394 |
| 1243 | GCA_000644075.1 | 1422617 | 1280 | Staphylococcus aureus VET0373R | VET0373R | Firmicutes | KK233916.1 | 3 | KAE45866.1 | 16397 |
| 1244 | GCA_000644095.1 | 1422618 | 1280 | Staphylococcus aureus VET0376R | VET0376R | Firmicutes | KK233953.1 | 3 | KAE50291.1 | 16400 |
| 1245 | GCA_000644115.1 | 1422619 | 1280 | Staphylococcus aureus VET0382R | VET0382R | Firmicutes | KK233998.1 | 3 | KAE51871.1 | 16403 |
| 1246 | GCA_000644135.1 | 1422620 | 1280 | Staphylococcus aureus VET0383R | VET0383R | Firmicutes | KK234034.1 | 3 | KAE56033.1 | 16406 |
| 1247 | GCA_000644155.1 | 1422621 | 1280 | Staphylococcus aureus VET0384R | VET0384R | Firmicutes | KK234091.1 | 3 | KAE58619.1 | 16409 |
| 1248 | GCA_000644175.1 | 1422622 | 1280 | Staphylococcus aureus VET0387R | VET0387R | Firmicutes | KK234154.1 | 3 | KAE60715.1 | 16412 |
| 1249 | GCA_000644195.1 | 1422623 | 1280 | Staphylococcus aureus VET0388R | VET0388R | Firmicutes | KK234207.1 | 3 | KAE63797.1 | 16415 |
| 1250 | GCA_000644215.1 | 1422624 | 1280 | Staphylococcus aureus VET0389R | VET0389R | Firmicutes | KK234244.1 | 3 | KAE63594.1 | 16418 |
| 1251 | GCA_000644235.1 | 1422625 | 1280 | Staphylococcus aureus VET0390R | VET0390R | Firmicutes | KK234279.1 | 3 | KAE68676.1 | 16421 |
| 1252 | GCA_000644255.1 | 1422626 | 1280 | Staphylococcus aureus VET0393R | VET0393R | Firmicutes | KK234337.1 | 3 | KAE69670.1 | 16424 |
| 1253 | GCA_000644275.1 | 1422627 | 1280 | Staphylococcus aureus VET0394R | VET0394R | Firmicutes | KK234392.1 | 3 | KAE72491.1 | 16427 |
| 1254 | GCA_000644295.1 | 1422628 | 1280 | Staphylococcus aureus VET0395R | VET0395R | Firmicutes | KK234444.1 | 3 | KAE78484.1 | 16430 |
| 1255 | GCA_000644315.1 | 1422629 | 1280 | Staphylococcus aureus VET0396R | VET0396R | Firmicutes | KK234517.1 | 3 | KAE76736.1 | 16433 |
| 1256 | GCA_000644335.1 | 1422630 | 1280 | Staphylococcus aureus VET0399R | VET0399R | Firmicutes | KK234558.1 | 3 | KAE81551.1 | 16436 |
| 1257 | GCA_000644355.1 | 1422632 | 1280 | Staphylococcus aureus VET0401R | VET0401R | Firmicutes | KK234614.1 | 3 | KAE84897.1 | 16439 |
| 1258 | GCA_000644375.1 | 1422633 | 1280 | Staphylococcus aureus VET0402R | VET0402R | Firmicutes | KK234667.1 | 3 | KAE84991.1 | 16442 |
| 1259 | GCA_000644395.1 | 1422634 | 1280 | Staphylococcus aureus VET0403R | VET0403R | Firmicutes | KK234707.1 | 3 | KAE89404.1 | 16445 |
| 1260 | GCA_000644415.1 | 1422635 | 1280 | Staphylococcus aureus VET0406R | VET0406R | Firmicutes | KK234760.1 | 3 | KAE90296.1 | 16448 |
| 1261 | GCA_000644435.1 | 1422636 | 1280 | Staphylococcus aureus VET0407R | VET0407R | Firmicutes | KK234801.1 | 3 | KAE93402.1 | 16451 |
| 1262 | GCA_000644455.1 | 1422637 | 1280 | Staphylococcus aureus VET0410R | VET0410R | Firmicutes | KK234843.1 | 3 | KAE97233.1 | 16454 |
| 1263 | GCA_000644475.1 | 1422638 | 1280 | Staphylococcus aureus VET0411R | VET0411R | Firmicutes | KK234874.1 | 3 | KAE99814.1 | 16457 |
| 1264 | GCA_000644495.1 | 1422639 | 1280 | Staphylococcus aureus VET0412R | VET0412R | Firmicutes | KK234915.1 | 3 | KAE99887.1 | 16460 |
| 1265 | GCA_000644515.1 | 1422640 | 1280 | Staphylococcus aureus VET0413R | VET0413R | Firmicutes | KK234944.1 | 3 | KAF04816.1 | 16463 |
| 1266 | GCA_000644535.1 | 1422642 | 1280 | Staphylococcus aureus VET0415R | VET0415R | Firmicutes | KK234978.1 | 3 | KAF09789.1 | 16466 |
| 1267 | GCA_000644555.1 | 1422643 | 1280 | Staphylococcus aureus VET0417R | VET0417R | Firmicutes | KK235031.1 | 3 | KAF06948.1 | 16469 |
| 1268 | GCA_000644575.1 | 1422644 | 1280 | Staphylococcus aureus VET0418R | VET0418R | Firmicutes | KK235067.1 | 3 | KAF12149.1 | 16472 |
| 1269 | GCA_000644595.1 | 1422645 | 1280 | Staphylococcus aureus VET0421R | VET0421R | Firmicutes | KK235112.1 | 3 | KAF14362.1 | 16475 |
| 1270 | GCA_000644615.1 | 1422647 | 1280 | Staphylococcus aureus VET0423R | VET0423R | Firmicutes | KK235151.1 | 3 | KAF17950.1 | 16478 |
| 1271 | GCA_000644635.1 | 1422649 | 1280 | Staphylococcus aureus VET0425R | VET0425R | Firmicutes | KK235201.1 | 3 | KAF21049.1 | 16481 |
| 1272 | GCA_000644655.1 | 1422651 | 1280 | Staphylococcus aureus VET0427R | VET0427R | Firmicutes | KK235253.1 | 3 | KAF20976.1 | 16484 |
| 1273 | GCA_000644675.1 | 1422652 | 1280 | Staphylococcus aureus VET0428R | VET0428R | Firmicutes | KK235284.1 | 3 | KAF25643.1 | 16487 |
| 1274 | GCA_000644695.1 | 1422653 | 1280 | Staphylococcus aureus VET0431R | VET0431R | Firmicutes | KK235319.1 | 3 | KAF31108.1 | 16490 |
| 1275 | GCA_000644715.1 | 1422654 | 1280 | Staphylococcus aureus VET0432R | VET0432R | Firmicutes | KK235373.1 | 3 | KAF28014.1 | 16493 |
| 1276 | GCA_000644735.1 | 1422655 | 1280 | Staphylococcus aureus VET0433R | VET0433R | Firmicutes | KK235408.1 | 3 | KAF33774.1 | 16496 |
| 1277 | GCA_000644755.1 | 1422656 | 1280 | Staphylococcus aureus VET0434R | VET0434R | Firmicutes | KK235447.1 | 3 | KAF35730.1 | 16499 |
| 1278 | GCA_000644775.1 | 1422657 | 1280 | Staphylococcus aureus VET0435R | VET0435R | Firmicutes | KK235486.1 | 3 | KAF38655.1 | 16502 |
| 1279 | GCA_000644795.1 | 1422660 | 1280 | Staphylococcus aureus VET0438R | VET0438R | Firmicutes | KK235526.1 | 3 | KAF42882.1 | 16505 |
| 1280 | GCA_000644815.1 | 1422661 | 1280 | Staphylococcus aureus VET0439R | VET0439R | Firmicutes | KK235571.1 | 3 | KAF42846.1 | 16508 |
| 1281 | GCA_000644835.1 | 1422662 | 1280 | Staphylococcus aureus VET0440R | VET0440R | Firmicutes | KK235606.1 | 3 | KAF46909.1 | 16511 |
| 1282 | GCA_000644855.1 | 1422663 | 1280 | Staphylococcus aureus VET0442R | VET0442R | Firmicutes | KK235649.1 | 3 | KAF47443.1 | 16514 |
| 1283 | GCA_000644875.1 | 1422664 | 1280 | Staphylococcus aureus VET0443R | VET0443R | Firmicutes | KK235707.1 | 3 | KAF48000.1 | 16517 |
| 1284 | GCA_000644895.1 | 1422665 | 1280 | Staphylococcus aureus VET0444R | VET0444R | Firmicutes | KK235727.1 | 3 | KAF54049.1 | 16520 |
| 1285 | GCA_000644915.1 | 1422666 | 1280 | Staphylococcus aureus VET0446R | VET0446R | Firmicutes | KK235765.1 | 3 | KAF54414.1 | 16523 |
| 1286 | GCA_000644935.1 | 1422667 | 1280 | Staphylococcus aureus VET0451R | VET0451R | Firmicutes | KK235791.1 | 3 | KAF60186.1 | 16526 |
| 1287 | GCA_000644955.1 | 1422668 | 1280 | Staphylococcus aureus VET0452R | VET0452R | Firmicutes | KK235845.1 | 3 | KAF62547.1 | 16529 |
| 1288 | GCA_000644975.1 | 1422669 | 1280 | Staphylococcus aureus VET0454R | VET0454R | Firmicutes | KK235896.1 | 3 | KAF64112.1 | 16532 |
| 1289 | GCA_000644995.1 | 1422670 | 1280 | Staphylococcus aureus VET0455R | VET0455R | Firmicutes | KK235952.1 | 3 | KAF67904.1 | 16535 |
| 1290 | GCA_000645015.1 | 1422671 | 1280 | Staphylococcus aureus VET0456R | VET0456R | Firmicutes | KK235994.1 | 3 | KAF70713.1 | 16538 |
| 1291 | GCA_000645035.1 | 1422672 | 1280 | Staphylococcus aureus VET0459R | VET0459R | Firmicutes | KK236040.1 | 3 | KAF71815.1 | 16541 |
| 1292 | GCA_000645055.1 | 1422673 | 1280 | Staphylococcus aureus VET0460R | VET0460R | Firmicutes | KK236088.1 | 3 | KAF75590.1 | 16544 |
| 1293 | GCA_000645075.1 | 1422674 | 1280 | Staphylococcus aureus VET0462R | VET0462R | Firmicutes | KK236132.1 | 3 | KAF76891.1 | 16547 |
| 1294 | GCA_000645095.1 | 1422675 | 1280 | Staphylococcus aureus VET0463R | VET0463R | Firmicutes | KK236171.1 | 3 | KAF80459.1 | 16550 |
| 1295 | GCA_000645115.1 | 1422676 | 1280 | Staphylococcus aureus VET0464R | VET0464R | Firmicutes | KK236208.1 | 3 | KAF84648.1 | 16553 |
| 1296 | GCA_000645155.1 | 1422679 | 1280 | Staphylococcus aureus VET0468R | VET0468R | Firmicutes | KK236266.1 | 3 | KAF88486.1 | 16556 |
| 1297 | GCA_000645175.1 | 1422681 | 1280 | Staphylococcus aureus VET0470R | VET0470R | Firmicutes | KK236311.1 | 3 | KAF92874.1 | 16559 |
| 1298 | GCA_000645195.1 | 1422685 | 1280 | Staphylococcus aureus VET0478R | VET0478R | Firmicutes | KK236370.1 | 3 | KAF91778.1 | 16562 |
| 1299 | GCA_000645215.1 | 1422686 | 1280 | Staphylococcus aureus VET0481R | VET0481R | Firmicutes | KK236396.1 | 3 | KAG01061.1 | 16565 |
| 1300 | GCA_000645235.1 | 1422687 | 1280 | Staphylococcus aureus VET0485R | VET0485R | Firmicutes | KK236455.1 | 3 | KAF98702.1 | 16568 |
| 1301 | GCA_000645255.1 | 1422688 | 1280 | Staphylococcus aureus VET0486R | VET0486R | Firmicutes | KK236493.1 | 3 | KAF97456.1 | 16571 |
| 1302 | GCA_000645275.1 | 1422689 | 1280 | Staphylococcus aureus VET0488R | VET0488R | Firmicutes | KK236523.1 | 3 | KAG04155.1 | 16574 |
| 1303 | GCA_000645295.1 | 1422752 | 1280 | Staphylococcus aureus VET0519S | VET0519S | Firmicutes | KK236567.1 | 3 | KAG04820.1 | 16577 |
| 1304 | GCA_000645315.1 | 1422691 | 1280 | Staphylococcus aureus VET0596R | VET0596R | Firmicutes | KK236596.1 | 3 | KAG08556.1 | 16580 |
| 1305 | GCA_000645355.1 | 1422755 | 1280 | Staphylococcus aureus VET0645S | VET0645S | Firmicutes | KK236705.1 | 3 | KAG10681.1 | 16583 |
| 1306 | GCA_000645375.1 | 1422756 | 1280 | Staphylococcus aureus VET0657S | VET0657S | Firmicutes | KK236725.1 | 3 | KAG16001.1 | 16586 |
| 1307 | GCA_000645435.1 | 1422760 | 1280 | Staphylococcus aureus VET0779S | VET0779S | Firmicutes | KK236834.1 | 3 | KAG21726.1 | 16589 |
| 1308 | GCA_000645475.1 | 1422763 | 1280 | Staphylococcus aureus VET0809S | VET0809S | Firmicutes | KK236898.1 | 3 | KAG29030.1 | 16592 |
| 1309 | GCA_000645495.1 | 1422764 | 1280 | Staphylococcus aureus VET0810S | VET0810S | Firmicutes | KK236944.1 | 3 | KAG32270.1 | 16595 |
| 1310 | GCA_000645535.1 | 1422766 | 1280 | Staphylococcus aureus VET0919S | VET0919S | Firmicutes | KK237057.1 | 3 | KAG34711.1 | 16598 |
| 1311 | GCA_000645555.1 | 1422767 | 1280 | Staphylococcus aureus VET0920S | VET0920S | Firmicutes | KK237082.1 | 3 | KAG37987.1 | 16601 |
| 1312 | GCA_000645655.1 | 1422772 | 1280 | Staphylococcus aureus VET1035S | VET1035S | Firmicutes | KK237308.1 | 3 | KAG51662.1 | 16604 |
| 1313 | GCA_000645675.1 | 1422773 | 1280 | Staphylococcus aureus VET1048S | VET1048S | Firmicutes | KK237351.1 | 3 | KAG55493.1 | 16607 |
| 1314 | GCA_000645695.1 | 1422774 | 1280 | Staphylococcus aureus VET1052S | VET1052S | Firmicutes | KK237399.1 | 3 | KAG59146.1 | 16610 |
| 1315 | GCA_000645715.1 | 1422775 | 1280 | Staphylococcus aureus VET1054S | VET1054S | Firmicutes | KK237441.1 | 3 | KAG59708.1 | 16613 |
| 1316 | GCA_000645735.1 | 1422777 | 1280 | Staphylococcus aureus VET1068S | VET1068S | Firmicutes | KK237467.1 | 3 | KAG65359.1 | 16616 |
| 1317 | GCA_000645755.1 | 1422778 | 1280 | Staphylococcus aureus VET1088S | VET1088S | Firmicutes | KK237500.1 | 3 | KAG67528.1 | 16619 |
| 1318 | GCA_000645775.1 | 1422780 | 1280 | Staphylococcus aureus VET1104S | VET1104S | Firmicutes | KK237537.1 | 3 | KAG68215.1 | 16622 |
| 1319 | GCA_000645795.1 | 1422781 | 1280 | Staphylococcus aureus VET1168S | VET1168S | Firmicutes | KK237569.1 | 3 | KAG69693.1 | 16625 |
| 1320 | GCA_000645815.1 | 1422782 | 1280 | Staphylococcus aureus VET1181S | VET1181S | Firmicutes | KK237594.1 | 3 | KAG74736.1 | 16628 |
| 1321 | GCA_000645835.1 | 1422783 | 1280 | Staphylococcus aureus VET1245S | VET1245S | Firmicutes | KK237651.1 | 3 | KAG76839.1 | 16631 |
| 1322 | GCA_000645855.1 | 1422786 | 1280 | Staphylococcus aureus VET1411S | VET1411S | Firmicutes | KK237699.1 | 3 | KAG76108.1 | 16634 |
| 1323 | GCA_000645875.1 | 1422789 | 1280 | Staphylococcus aureus VET1434S | VET1434S | Firmicutes | KK237730.1 | 3 | KAG81176.1 | 16637 |
| 1324 | GCA_000645895.1 | 1422790 | 1280 | Staphylococcus aureus VET1468S | VET1468S | Firmicutes | KK237767.1 | 3 | KAG82430.1 | 16640 |
| 1325 | GCA_000645915.1 | 1422791 | 1280 | Staphylococcus aureus VET1495S | VET1495S | Firmicutes | KK237796.1 | 3 | KAG84392.1 | 16643 |
| 1326 | GCA_000645935.1 | 1422792 | 1280 | Staphylococcus aureus VET1499S | VET1499S | Firmicutes | KK237828.1 | 3 | KAG88853.1 | 16646 |
| 1327 | GCA_000645975.1 | 1422795 | 1280 | Staphylococcus aureus VET1519S | VET1519S | Firmicutes | KK237920.1 | 3 | KAG92816.1 | 16649 |
| 1328 | GCA_000645995.1 | 1422796 | 1280 | Staphylococcus aureus VET1526S | VET1526S | Firmicutes | KK237967.1 | 3 | KAG95315.1 | 16652 |
| 1329 | GCA_000646055.1 | 1422799 | 1280 | Staphylococcus aureus VET1727S | VET1727S | Firmicutes | KK238084.1 | 3 | KAH01283.1 | 16655 |
| 1330 | GCA_000646075.1 | 1422800 | 1280 | Staphylococcus aureus VET1738S | VET1738S | Firmicutes | KK238117.1 | 3 | KAH06869.1 | 16658 |
| 1331 | GCA_000646115.1 | 1422802 | 1280 | Staphylococcus aureus VET1816S | VET1816S | Firmicutes | KK238213.1 | 3 | KAH07514.1 | 16661 |
| 1332 | GCA_000646135.1 | 1422803 | 1280 | Staphylococcus aureus VET1817S | VET1817S | Firmicutes | KK238232.1 | 3 | KAH16053.1 | 16664 |
| 1333 | GCA_000646155.1 | 1422804 | 1280 | Staphylococcus aureus VET1822S | VET1822S | Firmicutes | KK238270.1 | 3 | KAH15238.1 | 16667 |
| 1334 | GCA_000646195.1 | 1422693 | 1280 | Staphylococcus aureus VET1832R | VET1832R | Firmicutes | KK238379.1 | 3 | KAH19617.1 | 16670 |
| 1335 | GCA_000646215.1 | 1422695 | 1280 | Staphylococcus aureus VET1834R | VET1834R | Firmicutes | KK238417.1 | 3 | KAH24557.1 | 16673 |
| 1336 | GCA_000646235.1 | 1422696 | 1280 | Staphylococcus aureus VET1835R | VET1835R | Firmicutes | KK238465.1 | 3 | KAH28158.1 | 16676 |
| 1337 | GCA_000646255.1 | 1422697 | 1280 | Staphylococcus aureus VET1838R | VET1838R | Firmicutes | KK238513.1 | 3 | KAH29215.1 | 16679 |
| 1338 | GCA_000646275.1 | 1422698 | 1280 | Staphylococcus aureus VET1839R | VET1839R | Firmicutes | KK238556.1 | 3 | KAH33224.1 | 16682 |
| 1339 | GCA_000646295.1 | 1422700 | 1280 | Staphylococcus aureus VET1843R | VET1843R | Firmicutes | KK238600.1 | 3 | KAH35673.1 | 16685 |
| 1340 | GCA_000646315.1 | 1422701 | 1280 | Staphylococcus aureus VET1844R | VET1844R | Firmicutes | KK238654.1 | 3 | KAH37682.1 | 16688 |
| 1341 | GCA_000646335.1 | 1422702 | 1280 | Staphylococcus aureus VET1845R | VET1845R | Firmicutes | KK238712.1 | 3 | KAH40130.1 | 16691 |
| 1342 | GCA_000646355.1 | 1422703 | 1280 | Staphylococcus aureus VET1846R | VET1846R | Firmicutes | KK238762.1 | 3 | KAH41312.1 | 16694 |
| 1343 | GCA_000646375.1 | 1422704 | 1280 | Staphylococcus aureus VET1848R | VET1848R | Firmicutes | KK238790.1 | 3 | KAH45888.1 | 16697 |
| 1344 | GCA_000646395.1 | 1422705 | 1280 | Staphylococcus aureus VET1849R | VET1849R | Firmicutes | KK238827.1 | 3 | KAH47354.1 | 16700 |
| 1345 | GCA_000646415.1 | 1422706 | 1280 | Staphylococcus aureus VET1850R | VET1850R | Firmicutes | KK238862.1 | 3 | KAH50036.1 | 16703 |
| 1346 | GCA_000646435.1 | 1422707 | 1280 | Staphylococcus aureus VET1851R | VET1851R | Firmicutes | KK238898.1 | 3 | KAH53958.1 | 16706 |
| 1347 | GCA_000646455.1 | 1422708 | 1280 | Staphylococcus aureus VET1852R | VET1852R | Firmicutes | KK238957.1 | 3 | KAH55298.1 | 16709 |
| 1348 | GCA_000646475.1 | 1422709 | 1280 | Staphylococcus aureus VET1853R | VET1853R | Firmicutes | KK239008.1 | 3 | KAH59147.1 | 16712 |
| 1349 | GCA_000646495.1 | 1422710 | 1280 | Staphylococcus aureus VET1854R | VET1854R | Firmicutes | KK239060.1 | 3 | KAH62179.1 | 16715 |
| 1350 | GCA_000646515.1 | 1422711 | 1280 | Staphylococcus aureus VET1855R | VET1855R | Firmicutes | KK239111.1 | 3 | KAH64795.1 | 16718 |
| 1351 | GCA_000646535.1 | 1422712 | 1280 | Staphylococcus aureus VET1856R | VET1856R | Firmicutes | KK239165.1 | 3 | KAH67715.1 | 16721 |
| 1352 | GCA_000646555.1 | 1422714 | 1280 | Staphylococcus aureus VET1859R | VET1859R | Firmicutes | KK239222.1 | 3 | KAH70236.1 | 16724 |
| 1353 | GCA_000646575.1 | 1422715 | 1280 | Staphylococcus aureus VET1860R | VET1860R | Firmicutes | KK239279.1 | 3 | KAH70953.1 | 16727 |
| 1354 | GCA_000646595.1 | 1422716 | 1280 | Staphylococcus aureus VET1861R | VET1861R | Firmicutes | KK239317.1 | 3 | KAH74942.1 | 16730 |
| 1355 | GCA_000646615.1 | 1422717 | 1280 | Staphylococcus aureus VET1865R | VET1865R | Firmicutes | KK239359.1 | 3 | KAH77221.1 | 16733 |
| 1356 | GCA_000646635.1 | 1422718 | 1280 | Staphylococcus aureus VET1866R | VET1866R | Firmicutes | KK239396.1 | 3 | KAH77148.1 | 16736 |
| 1357 | GCA_000646655.1 | 1422719 | 1280 | Staphylococcus aureus VET1867R | VET1867R | Firmicutes | KK239416.1 | 3 | KAH82036.1 | 16739 |
| 1358 | GCA_000646675.1 | 1422720 | 1280 | Staphylococcus aureus VET1868R | VET1868R | Firmicutes | KK239446.1 | 3 | KAH85417.1 | 16742 |
| 1359 | GCA_000646695.1 | 1422722 | 1280 | Staphylococcus aureus VET1871R | VET1871R | Firmicutes | KK239489.1 | 3 | KAH87605.1 | 16745 |
| 1360 | GCA_000646715.1 | 1422723 | 1280 | Staphylococcus aureus VET1872R | VET1872R | Firmicutes | KK239544.1 | 3 | KAH91094.1 | 16748 |
| 1361 | GCA_000646735.1 | 1422724 | 1280 | Staphylococcus aureus VET1873R | VET1873R | Firmicutes | KK239596.1 | 3 | KAH91685.1 | 16751 |
| 1362 | GCA_000646755.1 | 1422725 | 1280 | Staphylococcus aureus VET1875R | VET1875R | Firmicutes | KK239632.1 | 3 | KAH96192.1 | 16754 |
| 1363 | GCA_000646775.1 | 1422727 | 1280 | Staphylococcus aureus VET1877R | VET1877R | Firmicutes | KK239680.1 | 3 | KAH96017.1 | 16757 |
| 1364 | GCA_000646795.1 | 1422728 | 1280 | Staphylococcus aureus VET1880R | VET1880R | Firmicutes | KK239707.1 | 3 | KAI00778.1 | 16760 |
| 1365 | GCA_000646815.1 | 1422729 | 1280 | Staphylococcus aureus VET1884R | VET1884R | Firmicutes | KK239758.1 | 3 | KAI04126.1 | 16763 |
| 1366 | GCA_000646835.1 | 1422730 | 1280 | Staphylococcus aureus VET1886R | VET1886R | Firmicutes | KK239796.1 | 3 | KAI02997.1 | 16766 |
| 1367 | GCA_000646855.1 | 1422731 | 1280 | Staphylococcus aureus VET1890R | VET1890R | Firmicutes | KK239825.1 | 3 | KAI08748.1 | 16769 |
| 1368 | GCA_000646875.1 | 1422732 | 1280 | Staphylococcus aureus VET1892R | VET1892R | Firmicutes | KK239862.1 | 3 | KAI10610.1 | 16772 |
| 1369 | GCA_000646895.1 | 1422733 | 1280 | Staphylococcus aureus VET1893R | VET1893R | Firmicutes | KK239901.1 | 3 | KAI13050.1 | 16775 |
| 1370 | GCA_000646915.1 | 1422734 | 1280 | Staphylococcus aureus VET1895R | VET1895R | Firmicutes | KK239942.1 | 3 | KAI15462.1 | 16778 |
| 1371 | GCA_000646935.1 | 1422736 | 1280 | Staphylococcus aureus VET1897R | VET1897R | Firmicutes | KK239986.1 | 3 | KAI16289.1 | 16781 |
| 1372 | GCA_000646955.1 | 1422738 | 1280 | Staphylococcus aureus VET1899R | VET1899R | Firmicutes | KK240031.1 | 3 | KAI19041.1 | 16784 |
| 1373 | GCA_000646975.1 | 1422739 | 1280 | Staphylococcus aureus VET1901R | VET1901R | Firmicutes | KK240053.1 | 3 | KAI22534.1 | 16787 |
| 1374 | GCA_000646995.1 | 1422740 | 1280 | Staphylococcus aureus VET1904R | VET1904R | Firmicutes | KK240086.1 | 3 | KAI26859.1 | 16790 |
| 1375 | GCA_000647015.1 | 1422741 | 1280 | Staphylococcus aureus VET1905R | VET1905R | Firmicutes | KK240125.1 | 3 | KAI29886.1 | 16793 |
| 1376 | GCA_000647035.1 | 1422742 | 1280 | Staphylococcus aureus VET1906R | VET1906R | Firmicutes | KK240163.1 | 3 | KAI31280.1 | 16796 |
| 1377 | GCA_000647055.1 | 1422744 | 1280 | Staphylococcus aureus VET1908R | VET1908R | Firmicutes | KK240210.1 | 3 | KAI34386.1 | 16799 |
| 1378 | GCA_000647075.1 | 1422745 | 1280 | Staphylococcus aureus VET1909R | VET1909R | Firmicutes | KK240248.1 | 3 | KAI37686.1 | 16802 |
| 1379 | GCA_000647095.1 | 1422747 | 1280 | Staphylococcus aureus VET1911R | VET1911R | Firmicutes | KK240284.1 | 3 | KAI38933.1 | 16805 |
| 1380 | GCA_000647115.1 | 1422748 | 1280 | Staphylococcus aureus VET1912R | VET1912R | Firmicutes | KK240323.1 | 3 | KAI42594.1 | 16808 |
| 1381 | GCA_000647135.1 | 1422749 | 1280 | Staphylococcus aureus VET1913R | VET1913R | Firmicutes | KK240355.1 | 3 | KAI44808.1 | 16811 |
| 1382 | GCA_000647155.1 | 1422750 | 1280 | Staphylococcus aureus VET1914R | VET1914R | Firmicutes | KK240383.1 | 3 | KAI46182.1 | 16814 |
| 1383 | GCA_000647175.1 | 1422805 | 1280 | Staphylococcus aureus VET1917S | VET1917S | Firmicutes | KK240411.1 | 3 | KAI50314.1 | 16817 |
| 1384 | GCA_000647195.1 | 1422807 | 1280 | Staphylococcus aureus VET1923S | VET1923S | Firmicutes | KK240446.1 | 3 | KAI52601.1 | 16820 |
| 1385 | GCA_000647215.1 | 1422809 | 1280 | Staphylococcus aureus VET1949S | VET1949S | Firmicutes | KK240494.1 | 3 | KAI51895.1 | 16823 |
| 1386 | GCA_000647235.1 | 1422810 | 1280 | Staphylococcus aureus VET1950S | VET1950S | Firmicutes | KK240520.1 | 3 | KAI58512.1 | 16826 |
| 1387 | GCA_000647255.1 | 1422812 | 1280 | Staphylococcus aureus VET1956S | VET1956S | Firmicutes | KK240554.1 | 3 | KAI59070.1 | 16829 |
| 1388 | GCA_000647275.1 | 1422813 | 1280 | Staphylococcus aureus VET1959S | VET1959S | Firmicutes | KK240580.1 | 3 | KAI62095.1 | 16832 |
| 1389 | GCA_000647295.1 | 1413576 | 1280 | Staphylococcus aureus ZTA09/ | ZTA09/ | Firmicutes | KK240619.1 | 3 | KAI65038.1 | 16835 |
| | | | | 03576-8HST | 03576-8HST | | | | | |
| 1390 | GCA_000647315.1 | 1413582 | 1280 | Staphylococcus aureus ZTA09/ | ZTA09/ | Firmicutes | KK240655.1 | 3 | KAI67117.1 | 16838 |
| | | | | 03734-9HSA | 03734-9HSA | | | | | |
| 1391 | GCA_000647335.1 | 1413584 | 1280 | Staphylococcus aureus ZTA09/ | ZTA09/ | Firmicutes | KK240683.1 | 3 | KAI71118.1 | 16841 |
| | | | | 03745-9HSA | 03745-9HSA | | | | | |
| 1392 | GCA_000647355.1 | 1413570 | 1280 | Staphylococcus aureus ZTA10/ | ZTA10/ | Firmicutes | KK240724.1 | 3 | KAI72298.1 | 16844 |
| | | | | 00045-9HST | 00045-9HST | | | | | |
| 1393 | GCA_000647375.1 | 1413574 | 1280 | Staphylococcus aureus ZTA10/ | ZTA10/ | Firmicutes | KK240757.1 | 3 | KAI76028.1 | 16847 |
| | | | | 00047-8HST | 00047-8HST | | | | | |
| 1394 | GCA_000647395.1 | 1413572 | 1280 | Staphylococcus aureus ZTA10/ | ZTA10/ | Firmicutes | KK240801.1 | 3 | KAI77491.1 | 16850 |
| | | | | 00058-8HST | 00058-8HST | | | | | |
| 1395 | GCA_000647415.1 | 1413581 | 1280 | Staphylococcus aureus ZTA10/ | ZTA10/ | Firmicutes | KK240836.1 | 3 | KAI79407.1 | 16853 |
| | | | | 02412-8HSA | 02412-8HSA | | | | | |
| 1396 | GCA_000647435.1 | 1413585 | 1280 | Staphylococcus aureus ZTA11/ | ZTA11/ | Firmicutes | KK240874.1 | 3 | KAI83421.1 | 16856 |
| | | | | 00189-8HSA | 00189-8HSA | | | | | |
| 1397 | GCA_000645135.1 | 1422677 | 1280 | Staphylococcus aureus VET0465R | VET0465R | Firmicutes | KK328149.1 | 3 | KAF82852.1 | 16859 |
| 1398 | GCA_000725445.1 | 1502784 | 1502784 | Pseudomonas sp. AAC | AAC | Proteobacteria | KL647094.1 | 3 | KES25284.1 | 16862 |
| 1399 | GCA_000969845.1 | 1078087 | 1078087 | Parabacteroides sp. HGS0025 | HGS0025 | Bacteroidetes | KQ033902.1 | 3 | KKB51170.1 | 16865 |
| 1400 | GCA_000969845.1 | 1078087 | 1078087 | Parabacteroides sp. HGS0025 | HGS0025 | Bacteroidetes | KQ033903.1 | 3 | KKB46310.1 | 16868 |
| 1401 | GCA_001032775.1 | 573 | 573 | Klebsiella pneumoniae | CHS201 | Proteobacteria | KQ088951.1 | 2 | KME80490.1 | 16871 |
| 1402 | GCA_001034405.1 | 615 | 615 | Serratia marcescens | UCI88 | Proteobacteria | KQ089789.1 | 2 | KMJ04347.1 | 16873 |
| 1403 | GCA_001457055.1 | 1414648 | 1414648 | Bacillus sp. SGD-V-76 | SGD-V-76 | Firmicutes | KQ758727.1 | 2 | KSU86125.1 | 16875 |
| 1404 | GCA_001519055.1 | 1739114 | 1739114 | Novosphingobium sp. Fuku2-ISO-50 | Fuku2-ISO-50 | Proteobacteria | KQ954285.1 | 4 | KUR80453.1 | 16877 |
| 1405 | GCA_001578705.1 | 1303 | 1303 | Streptococcus oralis | DD05 | Firmicutes | KQ969038.1 | 2 | KXT60079.1 | 16881 |
| 1406 | GCA_000956765.1 | 61645 | 61645 | Enterobacter asburiae | 33838 | Proteobacteria | LAAP01000054.1 | 3 | KJP12011.1 | 16883 |
| 1407 | GCA_000987705.1 | 1639 | 1639 | Listeria monocytogenes | DPC6895 | Firmicutes | LABG01000005.1 | 2 | KK042008.1 | 16886 |
| 1408 | GCA_001044655.1 | 1396 | 1396 | Bacillus cereus | INRA A3 | Firmicutes | LABH01000008.1 | 2 | KMQ31911.1 | 16888 |
| 1409 | GCA_001044655.1 | 1396 | 1396 | Bacillus cereus | INRA A3 | Firmicutes | LABH01000014.1 | 2 | KMQ31454.1 | 16890 |
| 1410 | GCA_001044655.1 | 1396 | 1396 | Bacillus cereus | INRA A3 | Firmicutes | LABH01000016.1 | 2 | KMQ31339.1 | 16892 |
| 1411 | GCA_001044855.1 | 1396 | 1396 | Bacillus cereus | RIVM BC 934 | Firmicutes | LABP01000126.1 | 2 | KMQ07576.1 | 16894 |
| 1412 | GCA_000963235.1 | 29290 | 29290 | Candidatus Magnetobacterium bavaricum | | Nitrospirae | LACI01000945.1 | 2 | KJU85605.1 | 16896 |
| 1413 | GCA_000961765.1 | 1629719 | 1629719 | Clostridiaceae bacterium BRH_c20a | | Firmicutes | LADT01000090.1 | 3 | KJS18752.1 | 16898 |
| 1414 | GCA_001010195.1 | 562 | 562 | Escherichia coli | M17-1 | Proteobacteria | LBDD01000010.1 | 2 | KLD44466.1 | 16901 |
| 1415 | GCA_001007525.1 | 670 | 670 | Vibrio parahaemolyticus | MAVP-E | Proteobacteria | LBHP01000003.1 | 3 | KKZ08483.1 | 16903 |
| 1416 | GCA_001005805.1 | 1590 | 1590 | Lactobacillus plantarum | PS128 | Firmicutes | LBHS01000003.1 | 2 | KKX43503.1 | 16906 |
| 1417 | GCA_000981945.1 | 1409947 | 1352 | Enterococcus faecium MRSN 3418 | MRSN 3418 | Firmicutes | LBIM01000096.1 | 3 | KKJ66889.1 | 16908 |
| 1418 | GCA_000996715.1 | 1618997 | 1618997 | Candidatus Uhrbacteria bacterium | | Candidatus | LCAU01000021.1 | 3 | KKR96940.1 | 16911 |
| | | | | GW2011_GWF2_41_16 | | Uhrbacteria | | | | |
| 1419 | GCA_001007555.1 | 615 | 615 | Serratia marcescens | 90-166 | Proteobacteria | LCWI01000023.1 | 2 | KKZ15776.1 | 16914 |
| 1420 | GCA_001008595.1 | 1396 | 1396 | Bacillus cereus | B4080 | Firmicutes | LCYK01000007.1 | 2 | KLA35044.1 | 16916 |
| 1421 | GCA_001008665.1 | 1396 | 1396 | Bacillus cereus | B4158 | Firmicutes | LCYP01000035.1 | 2 | KLA36292.1 | 16918 |
| 1422 | GCA_001011775.1 | 550 | 550 | Enterobacter cloacae | GN02032 | Proteobacteria | LDCC01000090.1 | 2 | KLF79538.1 | 16920 |
| 1423 | GCA_001011075.1 | 1656220 | 1656220 | Serratia sp. TEL | TEL | Proteobacteria | LDEG01000009.1 | 2 | KLE38479.1 | 16922 |
| 1424 | GCA_001043655.1 | 1396 | 1396 | Bacillus cereus | LK9 | Firmicutes | LDUP01000008.1 | 2 | KMN72583.1 | 16924 |
| 1425 | GCA_001043705.1 | 1628212 | 1628212 | Chromobacterium sp. LK11 | LK11 | Proteobacteria | LDUR01000028.1 | 4 | KMN78963.1 | 16926 |
| 1426 | GCA_001276465.1 | 61645 | 61645 | Enterobacter asburiae | C7 | Proteobacteria | LDVX01000157.1 | 3 | KOQ86134.1 | 16930 |
| 1427 | GCA_001276515.1 | 1661745 | 1661745 | Haemophilus sp. C1 | C1 | Proteobacteria | LDVZ01000018.1 | 3 | KOQ96629.1 | 16933 |
| 1428 | GCA_001580555.1 | 1609637 | 1609637 | Stenotrophomonas sp. DDT-1 | DDT-1 | Proteobacteria | LEKR01000030.1 | 3 | KXU94746.1 | 16936 |
| 1429 | GCA_001051865.1 | 211759 | 615 | Serratia marcescens subsp. marcescens | AH0650_Sm1 | Proteobacteria | LFJS01000014.1 | 2 | KMU49983.1 | 16939 |
| 1430 | GCA_001184405.1 | 1090471 | 1090471 | Candidatus Burkholderia humilis | UZHbot5 | Proteobacteria | LFMX01000038.1 | 3 | KMZ12177.1 | 16941 |
| 1431 | GCA_001183595.1 | 648 | 648 | Aeromonas caviae | A23 | Proteobacteria | LFX001000113.1 | 4 | KMY27377.1 | 16944 |
| 1432 | GCA_001270215.1 | 670 | 670 | Vibrio parahaemolyticus | S487-4 | Proteobacteria | LFZE01000076.1 | 3 | KOF39297.1 | 16948 |
| 1433 | GCA_001298135.1 | 405946 | 405946 | Oceanobacillus caeni | HM6 | Firmicutes | LGTK01000035.1 | 3 | KPH74233.1 | 16951 |
| 1434 | GCA_001297125.1 | 1690247 | 1690247 | Pseudomonas sp. RIT-PI-q | RIT-PI-q | Proteobacteria | LHPC01000026.1 | 3 | KPG99048.1 | 16954 |
| 1435 | GCA_001483145.1 | 172045 | 172045 | Elizabethkingia miricola | EM_CHUV | Bacteroidetes | LIQC01000017.1 | 4 | KUG12361.1 | 16957 |
| 1436 | GCA_001317205.1 | 211759 | 615 | Serratia marcescens subsp. marcescens | 945154301 | Proteobacteria | LJEV01000097.1 | 4 | KPS74519.1 | 16961 |
| 1437 | GCA_001317425.1 | 211759 | 615 | Serratia marcescens subsp. marcescens | 945174350 | Proteobacteria | LJEX01000059.1 | 4 | KPS95936.1 | 16965 |
| 1438 | GCA_001295775.1 | 1523428 | 1523428 | beta proteobacterium AAP99 | AAP99 | Proteobacteria | LJIA01000001.1 | 2 | KPF70566.1 | 16969 |
| 1439 | GCA_001411765.1 | 1255 | 1255 | Pediococcus pentosaceus | wikim20 | Firmicutes | LJIU01000002.1 | 2 | KQB82200.1 | 16971 |
| 1440 | GCA_001420605.1 | 1712029 | 1712029 | Bacillus sp. FJAT-25509 | FJAT-25509 | Firmicutes | LJIZ01000033.1 | 2 | KQL33557.1 | 16973 |
| 1441 | GCA_001401315.1 | 129140 | 251699 | Pseudomonas syringae pv. tagetis | ICMP4091 | Proteobacteria | LJRM01000029.1 | 4 | KPY89108.1 | 16975 |
| 1442 | GCA_001243675.1 | 1507977 | 1507977 | Vibrio sp. J2-4 | J2-4 | Proteobacteria | LK933622.1 | 3 | CDT62308.1 | 16979 |
| 1443 | GCA_001262155.1 | 1507978 | 1507978 | Vibrio sp. J2-26 | J2-26 | Proteobacteria | LK934170.1 | 3 | CDT49142.1 | 16982 |
| 1444 | GCA_001467105.1 | 1208100 | 317 | Pseudomonas syringae ICMP 19498 | ICMP 19498 | Proteobacteria | LKCH01000127.1 | 2 | KTC51736.1 | 16985 |
| 1445 | GCA_001466905.1 | 716914 | 716914 | Pseudomonas sp. ICMP 3272 | ICMP 3272 | Proteobacteria | LKEK01000086.1 | 2 | KTB70323.1 | 16987 |
| 1446 | GCA_001466845.1 | 1198306 | 317 | Pseudomonas syringae ICMP 11293 | ICMP 11293 | Proteobacteria | LKEP01000045.1 | 2 | KTB90078.1 | 16989 |
| 1447 | GCA_001467365.1 | 1198307 | 317 | Pseudomonas syringae ICMP 11292 | ICMP 11292 | Proteobacteria | LKGU01000007.1 | 2 | KTC03050.1 | 16991 |
| 1448 | GCA_001429565.1 | 180957 | 554 | Pectobacterium carotovorum subsp. | PcbHPI01 | Proteobacteria | LKKQ01000034.1 | 2 | KRF62306.1 | 16993 |
| | | | | brasiliense | | | | | | |
| 1449 | GCA_001449735.1 | 287 | 287 | Pseudomonas aeruginosa | WH-SGI-V-07184 | Proteobacteria | LLM001000014.1 | 2 | KSF14311.1 | 16995 |
| 1450 | GCA_001450755.1 | 287 | 287 | Pseudomonas aeruginosa | WH-SGI-V-07239 | Proteobacteria | LLNK01000023.1 | 5 | KSG00435.1 | 16997 |
| 1451 | GCA_001452525.1 | 287 | 287 | Pseudomonas aeruginosa | WH-SGI-V-07690 | Proteobacteria | LLSK01000113.1 | 5 | KSO26594.1 | 17002 |
| 1452 | GCA_001453015.1 | 287 | 287 | Pseudomonas aeruginosa | WH-SGI-V-07263 | Proteobacteria | LLTV01000002.1 | 5 | KSQ24404.1 | 17007 |
| 1453 | GCA_001453015.1 | 287 | 287 | Pseudomonas aeruginosa | WH-SGI-V-07263 | Proteobacteria | LLTV01000014.1 | 3 | KSQ18859.1 | 17012 |
| 1454 | GCA_001425705.1 | 1736430 | 1736430 | Pelomonas sp. Root1217 | Root1217 | Proteobacteria | LMDC01000012.1 | 2 | KQV52821.1 | 17015 |
| 1455 | GCA_001425835.1 | 1736436 | 1736436 | Afipia sp. Root123D2 | Rootl23D2 | Proteobacteria | LMDP01000003.1 | 2 | KQW19009.1 | 17017 |
| 1456 | GCA_001421745.1 | 1735694 | 1735694 | Sphingomonas sp. Leaf230 | Leaf230 | Proteobacteria | LMKW01000009.1 | 2 | KQN02369.1 | 17019 |
| 1457 | GCA_001424085.1 | 1736359 | 1736359 | Rhizobium sp. Leaf386 | Leaf386 | Proteobacteria | LMQF01000024.1 | 3 | KQS95552.1 | 17021 |
| 1458 | GCA_001428335.1 | 1736401 | 1736401 | Arthrobacter sp. Soil762 | Soil762 | Actinobacteria | LMSG01000001.1 | 2 | KRE80987.1 | 17024 |
| 1459 | GCA_000953375.1 | 40480 | 2095 | Mycoplasma capricolum subsp. capripneumoniae | F38 | Tenericutes | LN515398.1 | 2 | CEA11436.1 | 17026 |
| 1460 | GCA_000953235.1 | 40480 | 2095 | Mycoplasma capricolum subsp. capripneumoniae | ILRI181 | Tenericutes | LN515399.1 | 2 | CEA10432.1 | 17028 |
| 1461 | GCA_001460855.1 | 754345 | 715 | Actinobacillus pleuropneumoniae serovar 8 | | Proteobacteria | LN908249.1 | 3 | CUU52508.1 | 17030 |
| 1462 | GCA_001463205.1 | 562 | 562 | Escherichia coli | 50639799 | Proteobacteria | LNHQ01000124.1 | 2 | KSX76371.1 | 17033 |
| 1463 | GCA_001547355.1 | 1352 | 1352 | Enterococcus faecium | U-1313438 | Firmicutes | LNLE01000074.1 | 3 | KWY00933.1 | 17035 |
| 1464 | GCA_001545595.1 | 1352 | 1352 | Enterococcus faecium | VRE-1300937 | Firmicutes | LNLK01000231.1 | 3 | KWY16707.1 | 17038 |
| 1465 | GCA_001545605.1 | 1352 | 1352 | Enterococcus faecium | VRE-1400408 | Firmicutes | LNLP01000005.1 | 3 | KWY36996.1 | 17041 |
| 1466 | GCA_001547195.1 | 1352 | 1352 | Enterococcus faecium | VRE-1401878 | Firmicutes | LNLW01000317.1 | 2 | KWY51064.1 | 17044 |
| 1467 | GCA_001545785.1 | 1352 | 1352 | Enterococcus faecium | VRE-1402215 | Firmicutes | LNLZ01000131.1 | 2 | KWY60248.1 | 17046 |
| 1468 | GCA_001545675.1 | 1352 | 1352 | Enterococcus faecium | VRE-1402258 | Firmicutes | LNMC01000072.1 | 2 | KWY71636.1 | 17048 |
| 1469 | GCA_001547115.1 | 1352 | 1352 | Enterococcus faecium | VRE-1402435 | Firmicutes | LNME01000036.1 | 3 | KWY79778.1 | 17050 |
| 1470 | GCA_001547015.1 | 1352 | 1352 | Enterococcus faecium | VRE-1402673 | Firmicutes | LNMI01000065.1 | 2 | KWY89823.1 | 17053 |
| 1471 | GCA_001547025.1 | 1352 | 1352 | Enterococcus faecium | VRE-1402991 | Firmicutes | LNMJ01000050.1 | 3 | KWY92722.1 | 17055 |
| 1472 | GCA_001547045.1 | 1352 | 1352 | Enterococcus faecium | VRE-1403299 | Firmicutes | LNMK01000076.1 | 3 | KWY92992.1 | 17058 |
| 1473 | GCA_001547425.1 | 1352 | 1352 | Enterococcus faecium | VRE-1403540 | Firmicutes | LNMM01000174.1 | 3 | KWY96007.1 | 17061 |
| 1474 | GCA_001547445.1 | 1352 | 1352 | Enterococcus faecium | VRE-1404192 | Firmicutes | LNM001000097.1 | 3 | KWZ06304.1 | 17064 |
| 1475 | GCA_001455955.1 | 1280 | 1280 | Staphylococcus aureus | SA7112 | Firmicutes | LNTF01000010.1 | 3 | KST54505.1 | 17067 |
| 1476 | GCA_001467955.1 | 45072 | 45072 | Legionella quateirensis | ATCC 49507 | Proteobacteria | LNYR01000034.1 | 2 | KTD46365.1 | 17070 |
| 1477 | GCA_001515985.1 | 1734408 | 1734408 | Sphingomonadales bacterium BRH_c3 | | Proteobacteria | LOET01000087.1 | 2 | KUO54659.1 | 17072 |
| 1478 | GCA_001583705.1 | 1396 | 1396 | Bacillus cereus | FSL W8-0003 | Firmicutes | LOMQ01000001.1 | 2 | KXY14129.1 | 17074 |
| 1479 | GCA_001575545.1 | 562 | 562 | Escherichia coli | G213 | Proteobacteria | LOOH01000120.1 | 2 | KXQ35635.1 | 17076 |
| 1480 | GCA_001562375.1 | 562 | 562 | Escherichia coli | 108191 | Proteobacteria | LORE01000010.1 | 2 | KXG66578.1 | 17078 |
| 1481 | GCA_001527325.1 | 101571 | 101571 | Burkholderia ubonensis | MSMB2054 | Proteobacteria | LOZC01000019.1 | 4 | KVM18168.1 | 17080 |
| 1482 | GCA_001527345.1 | 101571 | 101571 | Burkholderia ubonensis | MSMB2055 | Proteobacteria | LOZD01000004.1 | 4 | KVM22194.1 | 17084 |
| 1483 | GCA_001527425.1 | 101571 | 101571 | Burkholderia ubonensis | MSMB2061 | Proteobacteria | LOZH01000020.1 | 4 | KVM54441.1 | 17088 |
| 1484 | GCA_001527955.1 | 101571 | 101571 | Burkholderia ubonensis | MSMB1599WGS | Proteobacteria | LPAJ01000129.1 | 2 | KVO16515.1 | 17092 |
| 1485 | GCA_001529565.1 | 101571 | 101571 | Burkholderia ubonensis | MSMB2022WGS | Proteobacteria | LPCI01000083.1 | 2 | KVR44248.1 | 17094 |
| 1486 | GCA_001528545.1 | 60552 | 60552 | Burkholderia vietnamiensis | AU25340 | Proteobacteria | LPCP01000011.1 | 3 | KVR99614.1 | 17096 |
| 1487 | GCA_001529645.1 | 60552 | 60552 | Burkholderia vietnamiensis | HI3392 | Proteobacteria | LPCS01000140.1 | 2 | KVS07629.1 | 17099 |
| 1488 | GCA_001529225.1 | 101571 | 101571 | Burkholderia ubonensis | MSMB1221WGS | Proteobacteria | LPEK01000051.1 | 3 | KVU94135.1 | 17101 |
| 1489 | GCA_001531435.1 | 101571 | 101571 | Burkholderia ubonensis | MSMB1508WGS | Proteobacteria | LPFT01000048.1 | 2 | KVX54694.1 | 17104 |
| 1490 | GCA_001531105.1 | 101571 | 101571 | Burkholderia ubonensis | MSMB2092WGS | Proteobacteria | LPHE01000088.1 | 3 | KWA77747.1 | 17106 |
| 1491 | GCA_001531535.1 | 101571 | 101571 | Burkholderia ubonensis | MSMB2093WGS | Proteobacteria | LPHF01000028.1 | 3 | KWB32211.1 | 17109 |
| 1492 | GCA_001532205.1 | 101571 | 101571 | Burkholderia ubonensis | MSMB2110WGS | Proteobacteria | LPHP01000047.1 | 2 | KWB80882.1 | 17112 |
| 1493 | GCA_001472685.1 | 301102 | 158836 | Enterobacter hormaechei subsp. oharae | SMART_723 | Proteobacteria | LPPG01000028.1 | 3 | KTI37619.1 | 17114 |
| 1494 | GCA_001472055.1 | 301102 | 158836 | Enterobacter hormaechei subsp. oharae | SMART_286 | Proteobacteria | LPQM01000012.1 | 2 | KTJ90670.1 | 17117 |
| 1495 | GCA_001572345.1 | 562 | 562 | Escherichia coli | CFSAN025116 | Proteobacteria | LPYQ01000233.1 | 2 | KXM51394.1 | 17119 |
| 1496 | GCA_001572325.1 | 562 | 562 | Escherichia coli | CFSAN025117 | Proteobacteria | LPYR01000272.1 | 2 | KXM54363.1 | 17121 |
| 1497 | GCA_001572445.1 | 562 | 562 | Escherichia coli | CFSAN025127 | Proteobacteria | LPYZ01000244.1 | 2 | KXM99995.1 | 17123 |
| 1498 | GCA_001563205.1 | 1352 | 1352 | Enterococcus faecium | 805447/07 | Firmicutes | LQRS01000049.1 | 3 | KXH57776.1 | 17125 |
| 1499 | GCA_001519675.1 | 562 | 562 | Escherichia coli | GN02137 | Proteobacteria | LQSE01000001.1 | 2 | KUS35848.1 | 17128 |
| 1500 | GCA_001521215.1 | 562 | 562 | Escherichia coli | GN02289 | Proteobacteria | LQSV01000130.1 | 2 | KUT10059.1 | 17130 |
| 1501 | GCA_001519835.1 | 562 | 562 | Escherichia coli | GN02290 | Proteobacteria | LQSW01000102.1 | 2 | KUT23043.1 | 17132 |
| 1502 | GCA_001521195.1 | 562 | 562 | Escherichia coli | GN02314 | Proteobacteria | LQSY01000192.1 | 2 | KUT23265.1 | 17134 |
| 1503 | GCA_001519855.1 | 562 | 562 | Escherichia coli | GN02317 | Proteobacteria | LQSZ01000228.1 | 2 | KUT30718.1 | 17136 |
| 1504 | GCA_001520275.1 | 562 | 562 | Escherichia coli | GN02359 | Proteobacteria | LQTG01000182.1 | 2 | KUT68247.1 | 17138 |
| 1505 | GCA_001520235.1 | 562 | 562 | Escherichia coli | GN02514 | Proteobacteria | LQUF01000113.1 | 2 | KUU91589.1 | 17140 |
| 1506 | GCA_001521115.1 | 562 | 562 | Escherichia coli | GN03661 | Proteobacteria | LQWC01000068.1 | 2 | KUX27115.1 | 17142 |
| 1507 | GCA_001518225.1 | 550 | 550 | Enterobacter cloacae | GN04225 | Proteobacteria | LRC001000001.1 | 2 | KUQ77231.1 | 17144 |
| 1508 | GCA_001518605.1 | 550 | 550 | Enterobacter cloacae | GN04369 | Proteobacteria | LRCT01000127.1 | 2 | KUQ90476.1 | 17146 |
| 1509 | GCA_001529365.1 | 56812 | 56812 | Shewanella frigidimarina | Ag06-30 | Proteobacteria | LRDC01000022.1 | 2 | KVX01473.1 | 17148 |
| 1510 | GCA_001524915.1 | 550 | 550 | Enterobacter cloacae | SMART_1260 | Proteobacteria | LRJW01000001.1 | 2 | KVK22109.1 | 17150 |
| 1511 | GCA_001604835.1 | 644 | 644 | Aeromonas hydrophila | AL09-79 | Proteobacteria | LRRV01000001.1 | 3 | KYQ15613.1 | 17152 |
| 1512 | GCA_001604855.1 | 644 | 644 | Aeromonas hydrophila | AL10-121 | Proteobacteria | LRRW01000002.1 | 3 | KYQ10111.1 | 17155 |
| 1513 | GCA_001604895.1 | 644 | 644 | Aeromonas hydrophila | ML09-121 | Proteobacteria | LRRX01000002.1 | 3 | KYQ13516.1 | 17158 |
| 1514 | GCA_001604905.1 | 644 | 644 | Aeromonas hydrophila | ML09-122 | Proteobacteria | LRRY01000003.1 | 3 | KYQ20474.1 | 17161 |
| 1515 | GCA_001548475.1 | 398518 | 103855 | Bordetella hinzii LMG 13501 | LMG 13501 | Proteobacteria | LRUJ01000004.1 | 2 | KXA74272.1 | 17164 |
| 1516 | GCA_000018545.1 | 394 | 380 | Sinorhizobium fredii NGR234 | NGR234 | Proteobacteria | U00090.2 | 2 | AAB91631.1 | 17166 |
| 1517 | GCA_000014225.1 | 342610 | 288 | Pseudoalteromonas atlantica T6c | T6c | Proteobacteria | CP000388.1 | 2 | ABG41254.1 | 17168 |
| 1518 | GCA_000022305.1 | 535289 | 721785 | Acidovorax ebreus TPSY | TPSY | Proteobacteria | CP001392.1 | 2 | ACM32747.1 | 17170 |
| 1519 | GCA_000146185.1 | 515620 | 39485 | [Eubacterium] eligens ATCC 27750 | ATCC 27750 | Firmicutes | CP001104.1 | 2 | ACR72776.1 | 17172 |
| 1520 | GCA_000020605.1 | 515619 | 39491 | [Eubacterium rectale] ATCC 33656 | ATCC 33656 | Firmicutes | CP001107.1 | 2 | ACR74433.1 | 17174 |
| 1521 | GCA_000190515.1 | 930170 | 1428 | Bacillus thuringiensis serovar | YBT-020 | Firmicutes | CP002509.1 | 2 | ADY24842.1 | 17176 |
| | | | | finitimus YBT-020 | | | | | | |
| 1522 | GCA_000262385.1 | 1126211 | 1390 | Bacillus amyloliquefaciens Y2 | Y2 | Firmicutes | CP003332.1 | 2 | AFJ62119.1 | 17178 |
| 1523 | GCA_000510285.1 | 1435044 | 76759 | Pseudomona monteiliis SB3078 | SB3078 | Proteobacteria | CP006978.1 | 2 | AHC83882.1 | 17180 |
| 1524 | GCA_000510325.1 | 1435058 | 76759 | Pseudomonas monteilii SB3101 | SB3101 | Proteobacteria | CP006979.1 | 2 | AHC89253.1 | 17182 |
| 1525 | GCA_000568815.1 | 1150621 | 66821 | Sulfurospirillum multivorans DSM 12446 | DSM 12446 | Proteobacteria | CP007201.1 | 2 | AHJ13792.1 | 17184 |
| 1526 | GCA_000746525.1 | 237609 | 237609 | Pseudomonas alkylphenolia | KL28 | Proteobacteria | CP009048.1 | 2 | AIL63104.1 | 17186 |
| 1527 | GCA_000784875.3 | 1484158 | 1484158 | Pantoea sp. PSNIH1 | PSNIH1 | Proteobacteria | CP009883.1 | 2 | AIX52585.1 | 17188 |
| 1528 | GCA_000784965.1 | 1484157 | 1484157 | Pantoea sp. PSNIH2 | PSNIH2 | Proteobacteria | CP009869.1 | 2 | AIX76375.1 | 17190 |
| 1529 | GCA_000827045.1 | 1570330 | 1570330 | Bacillus sp. BH072 | BH072 | Firmicutes | CP009938.1 | 2 | AJE80313.1 | 17192 |
| 1530 | GCA_001022195.1 | 571 | 571 | Klebsiella oxytoca | CAV1374 | Proteobacteria | CP011635.1 | 2 | AKL33255.1 | 17194 |
| 1531 | GCA_000009705.1 | 103690 | 103690 | Nostoc sp. PCC 7120 | | Cyanobacteria | BA000019.2 | 2 | BAB73898.1 | 17196 |
| 1532 | GCA_000009125.1 | 267608 | 305 | Ralstonia solanacearum GMI1000 | GMI1000 | Proteobacteria | AL646052.1 | 2 | CAD16319.1 | 17198 |
| 1533 | GCA_000196815.1 | 330214 | 330214 | Nitrospira defluvii | | Nitrospirae | FP929003.1 | 2 | CBK39910.1 | 17200 |
| 1534 | GCA_000284395.1 | 1155777 | 492670 | Bacillus methylotrophicus YAU B9601-Y2 | YAU B9601-Y2 | Firmicutes | HE774679.1 | 2 | CCG49955.1 | 17202 |
| 1535 | GCA_000438055.1 | 1262843 | 1262843 | Clostridium sp. CAG:813 | | Firmicutes | FR902242.1 | 2 | CDE97488.1 | 17204 |
| 1536 | GCA_000736655.1 | 1397851 | 40576 | Xenorhabdus bovienii str. feltiae France | feltiae France | Proteobacteria | HG426714.1 | 2 | CDG89550.1 | 17206 |
| 1537 | GCA_000736675.1 | 1397852 | 40576 | Xenorhabdus bovienii str. feltiae Florida | feltiae Florida | Proteobacteria | HG426895.1 | 2 | CDG92165.1 | 17208 |
| 1538 | GCA_000756695.1 | 1034943 | 1034943 | Legionella massiliensis | | Proteobacteria | CCSB01000001.1 | 2 | CDZ76665.1 | 17210 |
| 1539 | GCA_000756815.1 | 1034943 | 1034943 | Legionella massiliensis | LegA | Proteobacteria | CCVW01000001.1 | 2 | CEE12403.1 | 17212 |
| 1540 | GCA_001299055.1 | 1806504 | 1806504 | Achromobacter sp. 2789STDY5608606 | 2789STDY5608606 | Proteobacteria | CYTI01000029.1 | 2 | CUJ73685.1 | 17214 |
| 1541 | GCA_001538585.1 | 615 | 615 | Serratia marcescens | 2880STDY5682979 | Proteobacteria | FCLD01000012.1 | 2 | CVH06624.1 | 17216 |
| 1542 | GCA_001539465.1 | 615 | 615 | Serratia marcescens | 2880STDY5683000 | Proteobacteria | FCMT01000016.1 | 2 | CVH45990.1 | 17218 |
| 1543 | GCA_900064365.1 | 446 | 446 | Legionella pneumophila | 2532STDY5467650 | Proteobacteria | FJMH01000005.1 | 2 | CZP78225.1 | 17220 |
| 1544 | GCA_900064365.1 | 446 | 446 | Legionella pneumophila | 2532STDY5467650 | Proteobacteria | FJMH01000026.1 | 2 | CZQ07176.1 | 17222 |
| 1545 | GCA_000152545.1 | 350703 | 287 | Pseudomonas aeruginosa 2192 | 2192 | Proteobacteria | CH482384.1 | 2 | EAZ59087.1 | 17224 |
| 1546 | GCA_000173435.1 | 547043 | 28026 | Bifidobacterium pseudocatenulatum DSM 20438 = | DSM 20438 | Actinobacteria | ABXX02000002.1 | 2 | EEG71310.1 | 17226 |
| | | | | JCM 1200 = LMG 10505 | | | | | | |
| 1547 | GCA_000156535.1 | 536231 | 166486 | Roseburia intestinalis L1-82 | L1-82 | Firmicutes | GG692752.1 | 2 | EEU99049.1 | 17228 |
| 1548 | GCA_000162755.2 | 658187 | 168933 | Legionella drancourtii LLAP12 | LLAP12 | Proteobacteria | JH413795.1 | 2 | EHL32738.1 | 17230 |
| 1549 | GCA_000247895.1 | 883121 | 54291 | Raoultella ornithinolytica 10-5246 | 10-5246 | Proteobacteria | JH603146.1 | 2 | EHT09458.1 | 17232 |
| 1550 | GCA_000286415.1 | 1156919 | 72556 | Achromobacter piechaudii HLE | HLE | Proteobacteria | AUE01000146.1 | 2 | EJO27433.1 | 17234 |
| 1551 | GCA_000310225.1 | 1234382 | 164400 | Thauera phenylacetica B4P | B4P | Proteobacteria | AMXF01000005.1 | 2 | ENO98788.1 | 17236 |
| 1552 | GCA_000411195.1 | 883101 | 80866 | Delftia acidovorans CCUG 274B | CCUG 274B | Proteobacteria | KE150279.1 | 2 | EPD37371.1 | 17238 |
| 1553 | GCA_000466385.1 | 649747 | 1391 | Aneurinibacillus aneurinilyticus ATCC 12856 | ATCC 12856 | Firmicutes | KE952776.1 | 2 | ERI11820.1 | 17240 |
| 1554 | GCA_000481145.1 | 1402528 | 287 | Pseudomonas aeruginosa BWHPSA028 | BWHPSA028 | Proteobacteria | KI518959.1 | 2 | ERV92401.1 | 17242 |
| 1555 | GCA_000486525.1 | 930772 | 28901 | Salmonella enterica subsp. enterica | ATCC 43845 | Proteobacteria | AOXX01000056.1 | 2 | ESF54845.1 | 17244 |
| | | | | serovar Senftenberg str. ATCC 43845 | | | | | | |
| 1556 | GCA_000492715.1 | 1329844 | 550 | Enterobacter cloacae BWH 29 | BWH 29 | Proteobacteria | KI535567.1 | 2 | ESM46331.1 | 17246 |
| 1557 | GCA_000534175.1 | 1400151 | 550 | Enterobacter cloacae UCI 36 | UCI 36 | Proteobacteria | KI973125.1 | 2 | EUL65865.1 | 17248 |
| 1558 | GCA_000534195.1 | 1400150 | 550 | Enterobacter cloacae UCI 35 | UCI 35 | Proteobacteria | KI973132.1 | 2 | EUL69873.1 | 17250 |
| 1559 | GCA_000307775.2 | 1218352 | 316 | Pseudomonas stutzeri KOS6 | KOS6 | Proteobacteria | KK020676.1 | 2 | EWC39936.1 | 17252 |
| 1560 | GCA_000588875.1 | 1461752 | 1461752 | Sphingobium sp. Ant17 | Ant17 | Proteobacteria | JEMV01000093.1 | 2 | EXS68251.1 | 17254 |
| 1561 | GCA_000715205.1 | 1178540 | 1178540 | Bacillus sp. DW5-4 | DW5-4 | Firmicutes | JOTP01000043.1 | 2 | KEP24867.1 | 17256 |
| 1562 | GCA_000735105.1 | 421072 | 421072 | Epilithonimonas lactis | LMG 24401 | Bacteroidetes | JPLY01000003.1 | 2 | KFC21766.1 | 17258 |
| 1563 | GCA_000740715.1 | 1423 | 1423 | Bacillus subtilis | NKYL29 | Firmicutes | JPYY01000008.1 | 2 | KFI14244.1 | 17260 |
| 1564 | GCA_000741685.1 | 547043 | 28026 | Bifidobacterium pseudocatenulatum DSM | LMG 10505 | Actinobacteria | JGZF01000003.1 | 2 | KFI74923.1 | 17262 |
| | | | | 20438 = JCM 1200 = LMG 10505 | | | | | | |
| 1565 | GCA_000798515.1 | 562 | 562 | Escherichia coli | CVM N33561PS | Proteobacteria | JUDN01000122.1 | 2 | KHJ15951.1 | 17264 |
| 1566 | GCA_000935285.1 | 54291 | 54291 | Raoultella ornithinolytica | BAL286 | Proteobacteria | JXXF01000284.1 | 2 | KIZ39818.1 | 17266 |
| 1567 | GCA_000937505.2 | 546 | 546 | Citrobacter freundii | MRSN 12115 | Proteobacteria | JYFZ02000010.1 | 2 | KJB99792.1 | 17268 |
| 1568 | GCA_000957025.1 | 1619245 | 1619245 | Enterobacter sp. 35666 | 35666 | Proteobacteria | JZZC01000021.1 | 2 | KJN38560.1 | 17270 |
| 1569 | GCA_000964095.1 | 756892 | 756892 | Acinetobacter indicus | KM7 | Proteobacteria | JZRF01000069.1 | 2 | KJV39508.1 | 17272 |
| 1570 | GCA_001038915.1 | 293386 | 293386 | Bacillus stratosphericus | LK23 | Firmicutes | LDWL01000010.1 | 2 | KML18487.1 | 17274 |
| 1571 | GCA_001038775.1 | 293386 | 293386 | Bacillus stratosphericus | LK33 | Firmicutes | LDWU01000014.1 | 2 | KML62012.1 | 17276 |
| 1572 | GCA_001043535.1 | 293386 | 293386 | Bacillus stratosphericus | LK5 | Firmicutes | LDUH01000019.1 | 2 | KMN31317.1 | 17278 |
| 1573 | GCA_001043795.1 | 1628211 | 1628211 | Bacillus sp. LK10 | LK10 | Firmicutes | LDUQ01000030.1 | 2 | KMN70269.1 | 17280 |
| 1574 | GCA_001267695.1 | 1390 | 1390 | Bacillus amyloliquefaciens | KCTC 13012 | Firmicutes | LHCC01000003.1 | 2 | KOC82292.1 | 17282 |
| 1575 | GCA_001280655.1 | 670 | 670 | Vibrio parahaemolyticus | S372-5 | Proteobacteria | LIRQ01000039.1 | 2 | KOY23385.1 | 17284 |
| 1576 | GCA_001423425.1 | 1736330 | 1736330 | Rhizobium sp. Leaf306 | Leaf306 | Proteobacteria | LMNX01000001.1 | 2 | KQQ38326.1 | 17286 |
| 1577 | GCA_001431585.1 | 40324 | 40324 | Stenotrophomonas maltophilia | JCM9942 | Proteobacteria | LLXS01000084.1 | 2 | KRG37251.1 | 17288 |
| 1578 | GCA_001431425.1 | 83617 | 83617 | Stenotrophomonas nitritireducens | DSM 12575 | Proteobacteria | LDJG01000008.1 | 2 | KRG58645.1 | 17290 |
| 1579 | GCA_001449175.1 | 287 | 287 | Pseudomonas aeruginosa | WH-SGI-V-07074 | Proteobacteria | LLLA01000001.1 | 2 | KSC65530.1 | 17292 |
| 1580 | GCA_001452965.1 | 287 | 287 | Pseudomonas aeruginosa | WH-SGI-V-07260 | Proteobacteria | LLTT01000003.1 | 2 | KSQ38235.1 | 17294 |
| 1581 | GCA_001461845.1 | 1740805 | 1740805 | Bacillus sp. NRRL B-4257 | NRRL B-4257 | Firmicutes | LLZB01000026.1 | 2 | KSV97269.1 | 17296 |
| 1582 | GCA_001461825.1 | 1740803 | 1740803 | Bacillus sp. NRRL B-41580 | NRRL B-41580 | Firmicutes | LLZC01000021.1 | 2 | KSV97837.1 | 17298 |
| 1583 | GCA_001469685.1 | 1390 | 1390 | Bacillus amyloliquefaciens | Jxnuwx-1 | Firmicutes | LMAT01000001.1 | 2 | KTF62015.1 | 17300 |
| 1584 | GCA_001507425.1 | 1742859 | 1742859 | Flavobacteriaceae bacterium CRH | CRH | Bacteroidetes | LMAJ01000029.1 | 2 | KUJ59693.1 | 17302 |
| 1585 | GCA_001519075.1 | 1117702 | 1117702 | Novosphingobium fuchskuhlense | FNE08-7 | Proteobacteria | KQ954244.1 | 2 | KUR72680.1 | 17304 |
| 1586 | GCA_001526365.1 | 1637876 | 1637876 | Burkholderia sp. MSMB1835 | MSMB1835 | Proteobacteria | LOYP01000005.1 | 2 | KVL39772.1 | 17306 |
| 1587 | GCA_001593025.1 | 1671366 | 1671366 | Ruminococcus sp. DSM 100440 | DSM 100440 | Firmicutes | LFIL01000002.1 | 2 | KYG88423.1 | 17308 |
| 1588 | GCA_000013305.1 | 362663 | 562 | Escherichia coli 536 | 536 | Proteobacteria | CP000247.1 | 2 | ABG69918.1 | 17310 |
| 1589 | GCA_000333215.1 | 316435 | 562 | Escherichia coli Nissle 1917 | Nissle 1917 | Proteobacteria | CAPM01000056.1 | 2 | CCQ05514.1 | 17312 |
| 1590 | GCA_000627095.1 | 1444274 | 562 | Escherichia coli 1-182-04_S4_C3 | 1-182-04_S4_C3 | Proteobacteria | JJLH01000028.1 | 2 | EZJ21710.1 | 17314 |
| 1591 | GCA_000627215.1 | 1444245 | 562 | Escherichia coli 1-182-04_S4_C2 | 1-182-04_S4_C2 | Proteobacteria | JJLM01000058.1 | 2 | EZJ42018.1 | 17316 |
| 1592 | GCA_000627175.1 | 1444217 | 562 | Escherichia coli 1-182-04_S4_C1 | 1-182-04_S4_C1 | Proteobacteria | JJLP01000024.1 | 2 | EZJ61189.1 | 17318 |
| 1593 | GCA_000711455.1 | 1444214 | 562 | Escherichia coli 8-415-05_S4_C1 | 8-415-05_S4_C1 | Proteobacteria | JOMT01000106.1 | 2 | KEJ12238.1 | 17320 |
| 1594 | GCA_000711365.1 | 1444242 | 562 | Escherichia coli 8-415-05_S4_C2 | 8-415-05_S4_C2 | Proteobacteria | JOMU01000094.1 | 2 | KEJ13626.1 | 17322 |
| 1595 | GCA_000711435.1 | 1444271 | 562 | Escherichia coli 8-415-05_S4_C3 | 8-415-05_S4_C3 | Proteobacteria | JOMV01000018.1 | 2 | KEJ33279.1 | 17324 |
| 1596 | GCA_000713455.1 | 1444189 | 562 | Escherichia coli 8-415-05_S3_C3 | 8-415-05_S3_C3 | Proteobacteria | JOSA01000022.1 | 2 | KEN34070.1 | 17326 |
| 1597 | GCA_000713495.1 | 1444131 | 562 | Escherichia coli 8-415-05_S3_C1 | 8-415-05_S3_C1 | Proteobacteria | JOSB01000027.1 | 2 | KEN41712.1 | 17328 |
| 1598 | GCA_000713585.1 | 1444159 | 562 | Escherichia coli 8-415-05_S3_C2 | 8-415-05_S3_C2 | Proteobacteria | JOSI01000106.1 | 2 | KEN74069.1 | 17330 |
| 1599 | GCA_000194475.2 | 869685 | 562 | Escherichia coli 9.1649 | 9.1649 | Proteobacteria | AEZY02000025.1 | 2 | KKA60260.1 | 17332 |
| 1600 | GCA_000015425.1 | 400667 | 470 | Acinetobacter baumannii ATCC 17978 | ATCC 17978 | Proteobacteria | CP000521.1 | 2 | AB013066.2 | 17334 |
| 1601 | GCA_000410575.1 | 1331671 | 303 | Pseudomonas putida H8234 | H8234 | Proteobacteria | CP005976.1 | 2 | AGN81863.1 | 17336 |
| 1602 | GCA_000972745.1 | 339 | 339 | Xanthomonas campestris | 17 | Proteobacteria | CP011256.1 | 2 | AKC78200.1 | 17338 |
| 1603 | GCA_001587155.1 | 305 | 305 | Ralstonia solanacearum | IBSBF1503 | Proteobacteria | CP012943.1 | 2 | AMP74179.1 | 17340 |
| 1604 | GCA_000369305.1 | 1217628 | 470 | Acinetobacter baumannii NIPH 70 | NIPH 70 | Proteobacteria | KB849921.1 | 2 | ENW63934.1 | 17342 |
| 1605 | GCA_000967945.1 | 294 | 294 | Pseudomonas fluorescens | C1 | Proteobacteria | LACE01000014.1 | 2 | KJZ35756.1 | 17344 |
| 1606 | GCA_001452415.1 | 287 | 287 | Pseudomonas aeruginosa | WH-SGI-V-07681 | Proteobacteria | LLSB01000045.1 | 2 | KSM97145.1 | 17346 |
| 1607 | GCA_001593425.1 | 470 | 470 | Acinetobacter baumannii | ATCC 17978 | Proteobacteria | LOIA01000001.1 | 2 | KWX65128.1 | 17348 |
| 1608 | GCA_000965905.1 | 550 | 550 | Enterobacter cloacae | CIDEIMsCOL14 | Proteobacteria | JZKY01000004.1 | 2 | KJX33869.1 | 17350 |
| 1609 | GCA_000250075.2 | 868184 | 562 | Escherichia coli DEC10E | DEC10E | Proteobacteria | AIGT01000041.1 | 2 | EHW83928.1 | 17352 |
| 1610 | GCA_000250475.2 | 868204 | 562 | Escherichia coli DEC14D | DEC14D | Proteobacteria | AIHN01000055.1 | 2 | EHX90142.1 | 17354 |
| 1611 | GCA_000408605.1 | 1182702 | 562 | Escherichia coli KTE103 | KTE103 | Proteobacteria | KE137186.1 | 2 | EOW20700.1 | 17356 |
| 1612 | GCA_000458935.1 | 1281121 | 562 | Escherichia coli HVH 194 | HVH 194 | Proteobacteria | KE700788.1 | 2 | EQT99520.1 | 17358 |
| | | | | (4-2356805) | (4-2356805) | | | | | |
| 1613 | GCA_000488795.1 | 1269010 | 562 | Escherichia coli 910096-2 | 910096-2 | Proteobacteria | KI534966.1 | 2 | ESE28058.1 | 17360 |
| 1614 | GCA_000687105.1 | 1444147 | 562 | Escherichia coli 2-005-03_S3_C2 | 2-005-03_S3_C2 | Proteobacteria | JMGT01000049.1 | 2 | KDA72414.1 | 17362 |
| 1615 | GCA_000711415.1 | 1444071 | 562 | Escherichia coli 2-316-03_S1_C2 | 2-316-03_S1_C2 | Proteobacteria | JOMX01000122.1 | 2 | KEJ24822.1 | 17364 |
| 1616 | GCA_000711475.1 | 1444042 | 562 | Escherichia coli 2-316-03_S1_C1 | 2-316-03_S1_C1 | Proteobacteria | JOMW01000161.1 | 2 | KEJ23493.1 | 17366 |
| 1617 | GCA_000714105.1 | 1444239 | 562 | Escherichia coli 5-172-05_S4_C2 | 5-172-05_S4_C2 | Proteobacteria | JOQN01000018.1 | 2 | KEL24738.1 | 17368 |
| 1618 | GCA_000714145.1 | 1444211 | 562 | Escherichia coli 5-172-05_S4_C1 | 5-172-05_S4_C1 | Proteobacteria | JOQP01000002.1 | 2 | KEL41024.1 | 17370 |
| 1619 | GCA_000714225.1 | 1444269 | 562 | Escherichia coli 5-172-05_S4_C3 | 5-172-05_S4_C3 | Proteobacteria | JOQT01000008.1 | 2 | KEL61646.1 | 17372 |
| 1620 | GCA_000818965.1 | 1010817 | 562 | Escherichia coli C691-71 (14b) | C691-71 (14b) | Proteobacteria | JXBV01000029.1 | 2 | KIG27435.1 | 17374 |
| 1621 | GCA_001283365.1 | 562 | 562 | Escherichia coli | 503130_aEPEC | Proteobacteria | CYCS01000027.1 | 2 | CTV70059.1 | 17376 |
| 1622 | GCA_000492535.1 | 1328393 | 573 | Klebsiella pneumoniae UCICRE 7 | UCICRE 7 | Proteobacteria | KI535498.1 | 2 | ESL95857.1 | 17378 |
| 1623 | GCA_000688175.1 | 1438809 | 573 | Klebsiella pneumoniae UCI 64 | UCI 64 | Proteobacteria | JJNI01000059.1 | 2 | EZR09256.1 | 17380 |
| 1624 | GCA_001034055.1 | 573 | 573 | Klebsiella pneumoniae | BIDMC89 | Proteobacteria | KQ089634.1 | 2 | KMI15680.1 | 17382 |
| 1625 | GCA_000828775.1 | 1334564 | 615 | Serratia marcescens SM39 | SM39 | Proteobacteria | AP013063.1 | 2 | BAO34671.1 | 17384 |
| 1626 | GCA_001086525.1 | 630 | 630 | Yersinia enterocolitica | ERL08708 | Proteobacteria | CPZT01000005.1 | 2 | CNG20833.1 | 17386 |
| 1627 | GCA_001125285.1 | 630 | 630 | Yersinia enterocolitica | H1527/93 | Proteobacteria | CQCE01000004.1 | 2 | CNJ07176.1 | 17388 |
| 1628 | GCA_001515115.1 | 666 | 666 | Vibrio cholerae | M988 | Proteobacteria | LQBX01000042.1 | 2 | KUO23349.1 | 17390 |
| 1629 | GCA_000965785.1 | 817 | 817 | Bacteroides fragilis | BOB25 | Bacteroidetes | CP011073.1 | 2 | AKA50670.1 | 17392 |
| 1630 | GCA_000307435.1 | 999417 | 823 | Parabacteroides distasonis CL09T03C24 | CL09T03C24 | Bacteroidetes | JH976487.1 | 2 | EKN28634.1 | 17394 |
| 1631 | GCA_000156375.1 | 411464 | 901 | Desulfovibrio piger ATCC 29098 | ATCC 29098 | Proteobacteria | DS996355.1 | 2 | EEB34165.1 | 17396 |
| 1632 | GCA_000013885.1 | 323097 | 912 | Nitrobacter hamburgensis X14 | X14 | Proteobacteria | CP000319.1 | 2 | ABE61672.1 | 17398 |
| 1633 | GCA_000336555.1 | 883079 | 1034 | Afipia clevelandensis ATCC 49720 | ATCC 49720 | Proteobacteria | KB375281.1 | 2 | EKS39930.1 | 17400 |
| 1634 | GCA_000756355.1 | 1429888 | 1245 | Leuconostoc mesenteroides P45 | P45 | Firmicutes | JRGZ01000002.1 | 2 | KGB50927.1 | 17402 |
| 1635 | GCA_000146325.1 | 862514 | 1254 | Pediococcus acidilactici DSM 20284 | DSM 20284 | Firmicutes | GL397067.1 | 2 | EFL96228.1 | 17404 |
| 1636 | GCA_000195025.1 | 888813 | 1305 | Streptococcus sanguinis SK330 | SK330 | Firmicutes | GL878548.1 | 2 | EGF16080.1 | 17406 |
| 1637 | GCA_900059415.1 | 1307 | 1307 | Streptococcus suis | LSS12 | Firmicutes | FIFP01000007.1 | 2 | CYU22313.1 | 17408 |
| 1638 | GCA_900061375.1 | 1307 | 1307 | Streptococcus suis | SS985 | Firmicutes | FILR01000007.1 | 2 | CYX44081.1 | 17410 |
| 1639 | GCA_000339495.1 | 857115 | 1309 | Streptococcus mutans NLML8 | NLML8 | Firmicutes | AHSV01000017.1 | 2 | EMB57079.1 | 17412 |
| 1640 | GCA_000339955.1 | 857125 | 1309 | Streptococcus mutans N3209 | N3209 | Firmicutes | AHSL01000008.1 | 2 | EMC13032.1 | 17414 |
| 1641 | GCA_001558215.1 | 1309 | 1309 | Streptococcus mutans | NG8 | Firmicutes | CP013237.1 | 2 | AMF85252.1 | 17416 |
| 1642 | GCA_000186445.1 | 888745 | 1311 | Streptococcus agalactiae ATCC 13813 | ATCC 13813 | Firmicutes | GL636070.1 | 2 | EFV96552.1 | 17418 |
| 1643 | GCA_000221325.2 | 876138 | 1311 | Streptococcus agalactiae FSL S3-026 | FSLS3-026 | Firmicutes | AEXT01000002.1 | 2 | EGS28536.1 | 17420 |
| 1644 | GCA_000289795.1 | 1154788 | 1311 | Streptococcus agalactiae FSL S3-501 | FSLS3-501 | Firmicutes | ALQE01000108.1 | 2 | EPT39845.1 | 17422 |
| 1645 | GCA_000310365.1 | 1154776 | 1311 | Streptococcus agalactiae FSL S3-586 | FSLS3-586 | Firmicutes | ANCM01000071.1 | 2 | EPV87467.1 | 17424 |
| 1646 | GCA_000310425.1 | 1154786 | 1311 | Streptococcus agalactiae FSL S3-251 | FSLS3-251 | Firmicutes | ANC001000045.1 | 2 | EPV95019.1 | 17426 |
| 1647 | GCA_001017995.1 | 1311 | 1311 | Streptococcus agalactiae | DK-B-USS-155 | Firmicutes | LBKI01000029.1 | 2 | KLL29854.1 | 17428 |
| 1648 | GCA_001018015.1 | 1311 | 1311 | Streptococcus agalactiae | DK-B-USS-204 | Firmicutes | LBKK01000069.1 | 2 | KLL34442.1 | 17430 |
| 1649 | GCA_001018055.1 | 1311 | 1311 | Streptococcus agalactiae | DK-B-USS-215 | Firmicutes | LBKL01000103.1 | 2 | KLL34969.1 | 17432 |
| 1650 | GCA_000975495.1 | 1450185 | 1349 | Streptococcus uberis C8329 | C8329 | Firmicutes | JATG01000003.1 | 2 | KKF49829.1 | 17434 |
| 1651 | GCA_000205205.1 | 749517 | 1351 | Enterococcus faecalis TX1467 | TX1467 | Firmicutes | GL884006.1 | 2 | EGG58909.1 | 17436 |
| 1652 | GCA_000393495.1 | 1169246 | 1351 | Enterococcus faecalis EnGen0327 | A-3-1 | Firmicutes | KB944973.1 | 2 | EOL13670.1 | 17438 |
| 1653 | GCA_000393535.1 | 1169248 | 1351 | Enterococcus faecalis EnGen0329 | Merz192 | Firmicutes | KB945004.1 | 2 | EOK19568.1 | 17440 |
| 1654 | GCA_000395285.1 | 1158677 | 1351 | Enterococcus faecalis EnGen0289 | DS16 | Firmicutes | KB947440.1 | 2 | EOJ40184.1 | 17442 |
| 1655 | GCA_000395305.1 | 1158678 | 1351 | Enterococcus faecalis EnGen0285 | RC73 | Firmicutes | KB947448.1 | 2 | EOJ44767.1 | 17444 |
| 1656 | GCA_000415225.1 | 1258582 | 1351 | Enterococcus faecalis 20.SD.W.06 | 20.SD.W.06 | Firmicutes | KE351746.1 | 2 | EPI04163.1 | 17446 |
| 1657 | GCA_000519485.1 | 1391473 | 1351 | Enterococcus faecalis EnGen0403 | EnGen0403 | Firmicutes | KI912960.1 | 2 | ETT98569.1 | 17448 |
| 1658 | GCA_000519505.1 | 1391474 | 1351 | Enterococcus faecalis EnGen0404 | EnGen0404 | Firmicutes | KI912968.1 | 2 | ETU01533.1 | 17450 |
| 1659 | GCA_000519525.1 | 1391475 | 1351 | Enterococcus faecalis EnGen0405 | EnGen0405 | Firmicutes | KI912974.1 | 2 | ETU06238.1 | 17452 |
| 1660 | GCA_000519545.1 | 1391476 | 1351 | Enterococcus faecalis EnGen0406 | EnGen0406 | Firmicutes | KI912979.1 | 2 | ETU10204.1 | 17454 |
| 1661 | GCA_000519585.1 | 1391478 | 1351 | Enterococcus faecalis EnGen0408 | EnGen0408 | Firmicutes | KI912990.1 | 2 | ETU16615.1 | 17456 |
| 1662 | GCA_000519625.1 | 1391480 | 1351 | Enterococcus faecalis EnGen0410 | EnGen0410 | Firmicutes | KI913001.1 | 2 | ETU21340.1 | 17458 |
| 1663 | GCA_000519665.1 | 1391482 | 1351 | Enterococcus faecalis EnGen0412 | EnGen0412 | Firmicutes | KI913012.1 | 2 | ETU24458.1 | 17460 |
| 1664 | GCA_000519725.1 | 1391485 | 1351 | Enterococcus faecalis EnGen0415 | EnGen0415 | Firmicutes | KI913026.1 | 2 | ETU31995.1 | 17462 |
| 1665 | GCA_000519785.1 | 1391488 | 1351 | Enterococcus faecalis EnGen0418 | EnGen0418 | Firmicutes | KI913052.1 | 2 | ETU40537.1 | 17464 |
| 1666 | GCA_000519845.1 | 1391491 | 1351 | Enterococcus faecalis EnGen0421 | EnGen0421 | Firmicutes | KI913064.1 | 2 | ETU48375.1 | 17466 |
| 1667 | GCA_000442845.1 | 1137134 | 1358 | Lactococcus lactis subsp. lactis A12 | A12 | Firmicutes | CBLU010000009.1 | 2 | CDG04462.1 | 17468 |
| 1668 | GCA_001047375.1 | 1121307 | 1495 | Clostridium cylindrosporum DSM 605 | DSM 605 | Firmicutes | LFVU01000026.1 | 2 | KMT21891.1 | 17470 |
| 1669 | GCA_001579785.1 | 1502 | 1502 | Clostridium perfringens | JP838 | Firmicutes | CP010994.1 | 2 | AMN36763.1 | 17472 |
| 1670 | GCA_001372515.1 | 1505 | 1505 | [Clostridium] sordellii | W2922 | Firmicutes | CELK01000014.1 | 2 | CEQ11379.1 | 17474 |
| 1671 | GCA_000493655.1 | 1349417 | 1515 | Ruminiclostridium thermocellum BC1 | | Firmicutes | CBQ0010000050.1 | 2 | CDG36276.1 | 17476 |
| 1672 | GCA_000234155.1 | 742735 | 1531 | [Clostridium] clostridioforme 2_1_49FAA | 2_1_49FAA | Firmicutes | JH376870.1 | 2 | EHG33946.1 | 17478 |
| 1673 | GCA_000724405.2 | 1443118 | 1542 | Clostridium novyi A str. GD211209 | GD211209 | Firmicutes | JENN01000006.1 | 2 | KEH95234.1 | 17480 |
| 1674 | GCA_000469365.1 | 649758 | 1580 | Lactobacillus brevis ATCC 14869 = DSM 20054 | ATCC 14869 | Firmicutes | KI271248.1 | 2 | ERK44588.1 | 17482 |
| 1675 | GCA_001433855.1 | 649758 | 1580 | Lactobacillus brevis ATCC 14869 = DSM 20054 | DSM 20054 | Firmicutes | AZCP01000001.1 | 2 | KRK21385.1 | 17484 |
| 1676 | GCA_000175315.3 | 562973 | 1656 | Actinomyces viscosus C505 | C505 | Actinobacteria | KI391970.1 | 2 | EGE38623.2 | 17486 |
| 1677 | GCA_001602115.1 | 1748 | 1748 | Propionibacterium acidipropionici | ATCC 55737 | Actinobacteria | CP014352.1 | 2 | AMS06949.1 | 17488 |
| 1678 | GCA_000632085.1 | 13689 | 13689 | Sphingomonas paucimobilis | EPA505 | Proteobacteria | JFYY01000026.1 | 2 | EZP69577.1 | 17490 |
| 1679 | GCA_000955645.1 | 1347790 | 28131 | Prevotella intermedia ZT | ZT | Bacteroidetes | ATMK01000029.1 | 2 | KJJ86348.1 | 17492 |
| 1680 | GCA_000011545.1 | 272560 | 28450 | Burkholderia pseudomallei K96243 | K96243 | Proteobacteria | BX571965.1 | 2 | CAH34734.1 | 17494 |
| 1681 | GCA_000015925.1 | 357348 | 28450 | Burkholderia pseudomallei 1106a | 1106a | Proteobacteria | CP000572.1 | 2 | ABN90934.1 | 17496 |
| 1682 | GCA_000152345.1 | 331978 | 28450 | Burkholderia pseudomallei Pasteur 52237 | Pasteur 52237 | Proteobacteria | CH899746.1 | 2 | ED090949.1 | 17498 |
| 1683 | GCA_000193455.1 | 320371 | 28450 | Burkholderia pseudomallei 1710a | 1710a | Proteobacteria | CM000832.1 | 2 | EET07297.1 | 17500 |
| 1684 | GCA_000193475.1 | 357347 | 28450 | Burkholderia pseudomallei 1106b | 1106b | Proteobacteria | CM000774.1 | 2 | EES25793.1 | 17502 |
| 1685 | GCA_000259755.1 | 1086032 | 28450 | Burkholderia pseudomallei 1258a | 1258a | Proteobacteria | AHJB01000130.1 | 2 | EIF62858.1 | 17504 |
| 1686 | GCA_000259775.1 | 1086033 | 28450 | Burkholderia pseudomallei 1258b | 1258b | Proteobacteria | AHJC01000129.1 | 2 | EIF64459.1 | 17506 |
| 1687 | GCA_000259795.1 | 1086036 | 28450 | Burkholderia pseudomallei 354e | 354e | Proteobacteria | AHJD01000107.1 | 2 | EIF75597.1 | 17508 |
| 1688 | GCA_000259815.1 | 1086034 | 28450 | Burkholderia pseudomallei 354a | 354a | Proteobacteria | AGVS01000297.1 | 2 | EIF80279.1 | 17510 |
| 1689 | GCA_000294635.1 | 1229785 | 28450 | Burkholderia pseudomallei BPC006 | BPC006 | Proteobacteria | CP003781.1 | 2 | AFR14682.1 | 17512 |
| 1690 | GCA_000756125.1 | 28450 | 28450 | Burkholderia pseudomallei | Mahidol-1106a | Proteobacteria | CP008781.1 | 2 | AIO13383.1 | 17514 |
| 1691 | GCA_000756145.1 | 28450 | 28450 | Burkholderia pseudomallei | 1106a | Proteobacteria | CP008758.1 | 2 | AI091702.1 | 17516 |
| 1692 | GCA_000757035.2 | 331978 | 28450 | Burkholderia pseudomallei Pasteur 52237 | Pasteur 52237 | Proteobacteria | CP009899.1 | 2 | AJX61451.1 | 17518 |
| 1693 | GCA_000757185.1 | 28450 | 28450 | Burkholderia pseudomallei | BGS | Proteobacteria | KN150946.1 | 2 | KGC78979.1 | 17520 |
| 1694 | GCA_000757255.1 | 28450 | 28450 | Burkholderia pseudomallei | BGJ | Proteobacteria | KN150955.1 | 2 | KGD17915.1 | 17522 |
| 1695 | GCA_000763555.1 | 28450 | 28450 | Burkholderia pseudomallei | BGK | Proteobacteria | CP008916.1 | 2 | AIS47055.1 | 17524 |
| 1696 | GCA_000770455.1 | 1439854 | 28450 | Burkholderia pseudomallei B03 | B03 | Proteobacteria | CP009151.1 | 2 | AIV91806.1 | 17526 |
| 1697 | GCA_000770535.1 | 1439855 | 28450 | Burkholderia pseudomallei A79A | A79A | Proteobacteria | CP009165.1 | 2 | AIV95750.1 | 17528 |
| 1698 | GCA_000775085.1 | 1439860 | 28450 | Burkholderia pseudomallei TSV28 | TSV28 | Proteobacteria | JQHU01000150.1 | 2 | KGX68805.1 | 17530 |
| 1699 | GCA_000775675.1 | 1439856 | 28450 | Burkholderia pseudomallei A79C | A79C | Proteobacteria | JQHQ01000114.1 | 2 | KGY01694.1 | 17532 |
| 1700 | GCA_000775685.1 | 1439857 | 28450 | Burkholderia pseudomallei A79D | A79D | Proteobacteria | JQHR01000047.1 | 2 | KGY04116.1 | 17534 |
| 1701 | GCA_000932075.1 | 28450 | 28450 | Burkholderia pseudomallei | QCMRI_BP07 | Proteobacteria | JYBG01000039.1 | 2 | KIX42366.1 | 17536 |
| 1702 | GCA_000932145.1 | 28450 | 28450 | Burkholderia pseudomallei | QCMRI_BP32 | Proteobacteria | JYBK01000004.1 | 2 | KIX63305.1 | 17538 |
| 1703 | GCA_000953095.1 | 28450 | 28450 | Burkholderia pseudomallei | 3921 | Proteobacteria | LK936442.1 | 2 | CDU27413.1 | 17540 |
| 1704 | GCA_000954175.1 | 28450 | 28450 | Burkholderia pseudomallei | vgh07 | Proteobacteria | CP010973.1 | 2 | AJW52247.1 | 17542 |
| 1705 | GCA_000959285.1 | 272560 | 28450 | Burkholderia pseudomallei K96243 | K96243 | Proteobacteria | CP009538.1 | 2 | AJX28157.1 | 17544 |
| 1706 | GCA_001182265.1 | 28450 | 28450 | Burkholderia pseudomallei | EY6 | Proteobacteria | CWJD01000032.1 | 2 | CRY48213.1 | 17546 |
| 1707 | GCA_001191815.1 | 28450 | 28450 | Burkholderia pseudomallei | C32 | Proteobacteria | CSQC01000014.1 | 2 | CPN90195.1 | 17548 |
| 1708 | GCA_001192415.1 | 28450 | 28450 | Burkholderia pseudomallei | C36 | Proteobacteria | CSQF01000015.1 | 2 | CP007168.1 | 17550 |
| 1709 | GCA_001192815.1 | 28450 | 28450 | Burkholderia pseudomallei | C58 | Proteobacteria | CSOW01000015.1 | 2 | CPM40570.1 | 17552 |
| 1710 | GCA_001193035.1 | 28450 | 28450 | Burkholderia pseudomallei | C30 | Proteobacteria | CSSB01000014.1 | 2 | CPQ62517.1 | 17554 |
| 1711 | GCA_001193615.1 | 28450 | 28450 | Burkholderia pseudomallei | C11 | Proteobacteria | CSRL01000034.1 | 2 | CPQ18530.1 | 17556 |
| 1712 | GCA_001193655.1 | 28450 | 28450 | Burkholderia pseudomallei | C54 | Proteobacteria | CSLU01000014.1 | 2 | CPI70802.1 | 17558 |
| 1713 | GCA_001194765.1 | 28450 | 28450 | Burkholderia pseudomallei | C52 | Proteobacteria | CSN001000016.1 | 2 | CPK88012.1 | 17560 |
| 1714 | GCA_001196125.1 | 28450 | 28450 | Burkholderia pseudomallei | C31 | Proteobacteria | CSM001000014.1 | 2 | CPJ40971.1 | 17562 |
| 1715 | GCA_001196775.1 | 28450 | 28450 | Burkholderia pseudomallei | C50 | Proteobacteria | CSMS01000016.1 | 2 | CPJ76825.1 | 17564 |
| 1716 | GCA_001197015.1 | 28450 | 28450 | Burkholderia pseudomallei | C17 | Proteobacteria | CSMH01000029.1 | 2 | CPJ85555.1 | 17566 |
| 1717 | GCA_001197235.1 | 28450 | 28450 | Burkholderia pseudomallei | 6./96 | Proteobacteria | CSLAO1000038.1 | 2 | CPH77136.1 | 17568 |
| 1718 | GCA_001197775.1 | 28450 | 28450 | Burkholderia pseudomallei | C3 | Proteobacteria | CSME01000015.1 | 2 | CPJ25324.1 | 17570 |
| 1719 | GCA_001197795.1 | 28450 | 28450 | Burkholderia pseudomallei | C44 | Proteobacteria | CSSV01000015.1 | 2 | CPR02281.1 | 17572 |
| 1720 | GCA_001199195.1 | 28450 | 28450 | Burkholderia pseudomallei | C51 | Proteobacteria | CSMB01000033.1 | 2 | CPJ69674.1 | 17574 |
| 1721 | GCA_001199695.1 | 28450 | 28450 | Burkholderia pseudomallei | C68 | Proteobacteria | CSNM01000014.1 | 2 | CPK70853.1 | 17576 |
| 1722 | GCA_001199815.1 | 28450 | 28450 | Burkholderia pseudomallei | C28 | Proteobacteria | CSRV01000015.1 | 2 | CPQ22985.1 | 17578 |
| 1723 | GCA_001200615.1 | 28450 | 28450 | Burkholderia pseudomallei | C1 | Proteobacteria | CSPL01000013.1 | 2 | CPN03544.1 | 17580 |
| 1724 | GCA_001200935.1 | 28450 | 28450 | Burkholderia pseudomallei | 504/96 | Proteobacteria | CSLC01000034.1 | 2 | CPH78827.1 | 17582 |
| 1725 | GCA_001202415.1 | 28450 | 28450 | Burkholderia pseudomallei | C48 | Proteobacteria | CSLS01000014.1 | 2 | CPI57651.1 | 17584 |
| 1726 | GCA_001204515.1 | 28450 | 28450 | Burkholderia pseudomallei | C69 | Proteobacteria | CSPX01000005.1 | 2 | CPN26697.1 | 17586 |
| 1727 | GCA_001205935.1 | 28450 | 28450 | Burkholderia pseudomallei | C22 | Proteobacteria | CSNB01000034.1 | 2 | CPK20311.1 | 17588 |
| 1728 | GCA_001206495.1 | 28450 | 28450 | Burkholderia pseudomallei | C70 | Proteobacteria | CSOV01000015.1 | 2 | CPM33011.1 | 17590 |
| 1729 | GCA_001207675.1 | 28450 | 28450 | Burkholderia pseudomallei | C66 | Proteobacteria | CSQA01000015.1 | 2 | CPN87629.1 | 17592 |
| 1730 | GCA_001208545.1 | 28450 | 28450 | Burkholderia pseudomallei | C13 | Proteobacteria | CSSE01000014.1 | 2 | CPQ68104.1 | 17594 |
| 1731 | GCA_001209405.1 | 28450 | 28450 | Burkholderia pseudomallei | C15 | Proteobacteria | CSPA01000014.1 | 2 | CPM53772.1 | 17596 |
| 1732 | GCA_001209505.1 | 28450 | 28450 | Burkholderia pseudomallei | C21 | Proteobacteria | CSOU01000014.1 | 2 | CPM10513.1 | 17598 |
| 1733 | GCA_001210705.1 | 28450 | 28450 | Burkholderia pseudomallei | C16 | Proteobacteria | CSQP01000036.1 | 2 | CPP09133.1 | 17600 |
| 1734 | GCA_001210965.1 | 28450 | 28450 | Burkholderia pseudomallei | C42 | Proteobacteria | CSNW01000016.1 | 2 | CPL40888.1 | 17602 |
| 1735 | GCA_001211345.1 | 28450 | 28450 | Burkholderia pseudomallei | C62 | Proteobacteria | CSPM01000015.1 | 2 | CPN14436.1 | 17604 |
| 1736 | GCA_001211405.1 | 28450 | 28450 | Burkholderia pseudomallei | C26 | Proteobacteria | CSNQ01000016.1 | 2 | CPK90390.1 | 17606 |
| 1737 | GCA_001211505.1 | 28450 | 28450 | Burkholderia pseudomallei | C53 | Proteobacteria | CSRJ01000015.1 | 2 | CPP68451.1 | 17608 |
| 1738 | GCA_001211565.1 | 28450 | 28450 | Burkholderia pseudomallei | C10 | Proteobacteria | CSRH01000038.1 | 2 | CPP97990.1 | 17610 |
| 1739 | GCA_001211865.1 | 28450 | 28450 | Burkholderia pseudomallei | C29 | Proteobacteria | CSQB01000014.1 | 2 | CPN87378.1 | 17612 |
| 1740 | GCA_001212025.1 | 28450 | 28450 | Burkholderia pseudomallei | C2 | Proteobacteria | CSSW01000015.1 | 2 | CPR00121.1 | 17614 |
| 1741 | GCA_001212065.1 | 28450 | 28450 | Burkholderia pseudomallei | P6 | Proteobacteria | CSLI01000012.1 | 2 | CPH39453.1 | 17616 |
| 1742 | GCA_001212185.1 | 28450 | 28450 | Burkholderia pseudomallei | 21/96 | Proteobacteria | CSKV01000012.1 | 2 | CPG82753.1 | 17618 |
| 1743 | GCA_001212325.1 | 28450 | 28450 | Burkholderia pseudomallei | C25 | Proteobacteria | CSOX01000015.1 | 2 | CPM46611.1 | 17620 |
| 1744 | GCA_001212405.1 | 28450 | 28450 | Burkholderia pseudomallei | EY1 | Proteobacteria | CSKM01000009.1 | 2 | CPG38360.1 | 17622 |
| 1745 | GCA_001212465.1 | 28450 | 28450 | Burkholderia pseudomallei | 2/96 | Proteobacteria | CSKX01000014.1 | 2 | CPH00885.1 | 17624 |
| 1746 | GCA_001212705.1 | 28450 | 28450 | Burkholderia pseudomallei | EY3 | Proteobacteria | CSKP01000034.1 | 2 | CPH42028.1 | 17626 |
| 1747 | GCA_001277875.1 | 28450 | 28450 | Burkholderia pseudomallei | vgh16R | Proteobacteria | CP012515.1 | 2 | ALB92817.1 | 17628 |
| 1748 | GCA_001277895.1 | 28450 | 28450 | Burkholderia pseudomallei | vgh16W | Proteobacteria | CP012517.1 | 2 | ALB98879.1 | 17630 |
| 1749 | GCA_001277975.1 | 28450 | 28450 | Burkholderia pseudomallei | 982 | Proteobacteria | CP012576.1 | 2 | ALC58207.1 | 17632 |
| 1750 | GCA_001320215.1 | 28450 | 28450 | Burkholderia pseudomallei | 6 | Proteobacteria | CGEV01000013.1 | 2 | CFT55154.1 | 17634 |
| 1751 | GCA_001320825.1 | 28450 | 28450 | Burkholderia pseudomallei | C37 | Proteobacteria | CIHR01000013.1 | 2 | CPQ86668.1 | 17636 |
| 1752 | GCA_001320865.1 | 28450 | 28450 | Burkholderia pseudomallei | C61 | Proteobacteria | CFZT01000014.1 | 2 | CFP14331.1 | 17638 |
| 1753 | GCA_001320905.1 | 28450 | 28450 | Burkholderia pseudomallei | C6 | Proteobacteria | CGFU01000014.1 | 2 | CFU67050.1 | 17640 |
| 1754 | GCA_001320945.1 | 28450 | 28450 | Burkholderia pseudomallei | C4 | Proteobacteria | CFXK01000014.1 | 2 | CFM98899.1 | 17642 |
| 1755 | GCA_001320985.1 | 28450 | 28450 | Burkholderia pseudomallei | C9 | Proteobacteria | CFXG01000012.1 | 2 | CFM81858.1 | 17644 |
| 1756 | GCA_001321015.1 | 28450 | 28450 | Burkholderia pseudomallei | C7 | Proteobacteria | CIJT01000014.1 | 2 | CPM85720.1 | 17646 |
| 1757 | GCA_001321045.1 | 28450 | 28450 | Burkholderia pseudomallei | C8 | Proteobacteria | CIGV01000033.1 | 2 | CPL97294.1 | 17648 |
| 1758 | GCA_001321065.1 | 28450 | 28450 | Burkholderia pseudomallei | C12 | Proteobacteria | CFZL01000037.1 | 2 | CFO92224.1 | 17650 |
| 1759 | GCA_001321085.1 | 28450 | 28450 | Burkholderia pseudomallei | C46 | Proteobacteria | CFXP01000015.1 | 2 | CFN19260.1 | 17652 |
| 1760 | GCA_001321105.1 | 28450 | 28450 | Burkholderia pseudomallei | C45 | Proteobacteria | CIGX01000033.1 | 2 | CPL11643.1 | 17654 |
| 1761 | GCA_001321125.1 | 28450 | 28450 | Burkholderia pseudomallei | C43 | Proteobacteria | CIBE01000017.1 | 2 | CPN25051.1 | 17656 |
| 1762 | GCA_001321145.1 | 28450 | 28450 | Burkholderia pseudomallei | C47 | Proteobacteria | CIKM01000030.1 | 2 | CPO46736.1 | 17658 |
| 1763 | GCA_001321165.1 | 28450 | 28450 | Burkholderia pseudomallei | C27 | Proteobacteria | CFWL01000014.1 | 2 | CFL99481.1 | 17660 |
| 1764 | GCA_001321185.1 | 28450 | 28450 | Burkholderia pseudomallei | C24 | Proteobacteria | CIJQ01000014.1 | 2 | CP089903.1 | 17662 |
| 1765 | GCA_001321205.1 | 28450 | 28450 | Burkholderia pseudomallei | C23 | Proteobacteria | CGHT01000014.1 | 2 | CFW49159.1 | 17664 |
| 1766 | GCA_001321225.1 | 28450 | 28450 | Burkholderia pseudomallei | C64 | Proteobacteria | CFWQ01000015.1 | 2 | CFM22755.1 | 17666 |
| 1767 | GCA_001321245.1 | 28450 | 28450 | Burkholderia pseudomallei | C63 | Proteobacteria | CIBB01000014.1 | 2 | CPK84415.1 | 17668 |
| 1768 | GCA_001321265.1 | 28450 | 28450 | Burkholderia pseudomallei | C65 | Proteobacteria | CFZI01000014.1 | 2 | CFO79487.1 | 17670 |
| 1769 | GCA_001321285.1 | 28450 | 28450 | Burkholderia pseudomallei | C67 | Proteobacteria | CGFW01000014.1 | 2 | CFU75863.1 | 17672 |
| 1770 | GCA_001321305.1 | 28450 | 28450 | Burkholderia pseudomallei | C14 | Proteobacteria | CGFJ01000015.1 | 2 | CFU18823.1 | 17674 |
| 1771 | GCA_001321325.1 | 28450 | 28450 | Burkholderia pseudomallei | C18 | Proteobacteria | CFXB01000030.1 | 2 | CFM67594.1 | 17676 |
| 1772 | GCA_001321345.1 | 28450 | 28450 | Burkholderia pseudomallei | C20 | Proteobacteria | CGIB01000015.1 | 2 | CFW79173.1 | 17678 |
| 1773 | GCA_001321365.1 | 28450 | 28450 | Burkholderia pseudomallei | C19 | Proteobacteria | CFZB01000015.1 | 2 | CFO54185.1 | 17680 |
| 1774 | GCA_001321385.1 | 28450 | 28450 | Burkholderia pseudomallei | C49 | Proteobacteria | CFXU01000016.1 | 2 | CFN47247.1 | 17682 |
| 1775 | GCA_001321405.1 | 28450 | 28450 | Burkholderia pseudomallei | C57 | Proteobacteria | CGFL01000014.1 | 2 | CFU32908.1 | 17684 |
| 1776 | GCA_001321425.1 | 28450 | 28450 | Burkholderia pseudomallei | C59 | Proteobacteria | CFZV01000014.1 | 2 | CFP26209.1 | 17686 |
| 1777 | GCA_001321445.1 | 28450 | 28450 | Burkholderia pseudomallei | C60 | Proteobacteria | CIBL01000035.1 | 2 | CPP85431.1 | 17688 |
| 1778 | GCA_001321465.1 | 28450 | 28450 | Burkholderia pseudomallei | C41 | Proteobacteria | CFWT01000016.1 | 2 | CFM37245.1 | 17690 |
| 1779 | GCA_001321485.1 | 28450 | 28450 | Burkholderia pseudomallei | C56 | Proteobacteria | CIBF01000030.1 | 2 | CPQ63811.1 | 17692 |
| 1780 | GCA_001321505.1 | 28450 | 28450 | Burkholderia pseudomallei | C39 | Proteobacteria | CFYP01000014.1 | 2 | CF010819.1 | 17694 |
| 1781 | GCA_001321525.1 | 28450 | 28450 | Burkholderia pseudomallei | C40 | Proteobacteria | CIGS01000014.1 | 2 | CPL75788.1 | 17696 |
| 1782 | GCA_001321545.1 | 28450 | 28450 | Burkholderia pseudomallei | C38 | Proteobacteria | CFXD01000034.1 | 2 | CFM72557.1 | 17698 |
| 1783 | GCA_001321565.1 | 28450 | 28450 | Burkholderia pseudomallei | C35 | Proteobacteria | CIJK01000015.1 | 2 | CPK97832.1 | 17700 |
| 1784 | GCA_001321585.1 | 28450 | 28450 | Burkholderia pseudomallei | C33 | Proteobacteria | CHKY01000034.1 | 2 | CPN81614.1 | 17702 |
| 1785 | GCA_001321605.1 | 28450 | 28450 | Burkholderia pseudomallei | C34 | Proteobacteria | CGHK01000004.1 | 2 | CFW09147.1 | 17704 |
| 1786 | GCA_001321625.1 | 28450 | 28450 | Burkholderia pseudomallei | C55 | Proteobacteria | CGFR01000015.1 | 2 | CFU54707.1 | 17706 |
| 1787 | GCA_001326895.1 | 28450 | 28450 | Burkholderia pseudomallei | 2746b | Proteobacteria | CFVL01000010.1 | 2 | CFK50101.1 | 17708 |
| 1788 | GCA_001326935.1 | 28450 | 28450 | Burkholderia pseudomallei | EY4 | Proteobacteria | CHFD01000017.1 | 2 | CPG83473.1 | 17710 |
| 1789 | GCA_001327035.1 | 28450 | 28450 | Burkholderia pseudomallei | DB30729/00 | Proteobacteria | CGEZ01000012.1 | 2 | CFT72113.1 | 17712 |
| 1790 | GCA_001327075.1 | 28450 | 28450 | Burkholderia pseudomallei | 497/96 | Proteobacteria | CFVV01000013.1 | 2 | CFL13092.1 | 17714 |
| 1791 | GCA_001327155.1 | 28450 | 28450 | Burkholderia pseudomallei | 35/96 | Proteobacteria | CFWJ01000011.1 | 2 | CFL88486.1 | 17716 |
| 1792 | GCA_001327395.1 | 28450 | 28450 | Burkholderia pseudomallei | 708a | Proteobacteria | CIAY01000003.1 | 2 | CPE71818.1 | 17718 |
| 1793 | GCA_001327415.1 | 28450 | 28450 | Burkholderia pseudomallei | BRI | Proteobacteria | CGFB01000038.1 | 2 | CFT96170.1 | 17720 |
| 1794 | GCA_001327435.1 | 28450 | 28450 | Burkholderia pseudomallei | E8 | Proteobacteria | CGFC01000007.1 | 2 | CFT85447.1 | 17722 |
| 1795 | GCA_001327495.1 | 28450 | 28450 | Burkholderia pseudomallei | TRF661 | Proteobacteria | CFWE01000007.1 | 2 | CFL56334.1 | 17724 |
| 1796 | GCA_001327555.1 | 28450 | 28450 | Burkholderia pseudomallei | E0387 | Proteobacteria | CHFI01000014.1 | 2 | CPI51234.1 | 17726 |
| 1797 | GCA_000486815.1 | 997333 | 28901 | Salmonella enterica subsp. | 0250 | Proteobacteria | AOYM01000072.1 | 2 | ESG20657.1 | 17728 |
| | | | | enterica serovar Oranienburg | | | | | | |
| | | | | str. 0250 | | | | | | |
| 1798 | GCA_000940895.1 | 1454592 | 28901 | Salmonella enterica subsp. | CDC 06-0532 | Proteobacteria | CP007211.1 | 2 | AJQ64506.1 | 17730 |
| | | | | enterica serovar Anatum str. | | | | | | |
| | | | | CDC 06-0532 | | | | | | |
| 1799 | GCA_000963535.1 | 1454590 | 28901 | Salmonella enterica subsp. | USDA-ARS- | Proteobacteria | CP007483.1 | 2 | AJZ80320.1 | 17732 |
| | | | | enterica serovar Anatum str. | USMARC-1175 | | | | | |
| | | | | USDA-ARS-USMARC-1175 | | | | | | |
| 1800 | GCA_001217125.1 | 36809 | 36809 | Mycobacterium abscessus | PAP090 | Actinobacteria | CSWS01000008.1 | 2 | CPW02861.1 | 17734 |
| 1801 | GCA_000332975.1 | 1220583 | 36820 | Gordonia aichiensis NBRC 108223 | NBRC 108223 | Actinobacteria | BANR01000007.1 | 2 | GAC48719.1 | 17736 |
| 1802 | GCA_000014925.1 | 290340 | 43663 | Paenarthrobacter aurescens TC1 | TC1; ATCC | Actinobacteria | CP000474.1 | 2 | ABM09581.1 | 17738 |
| | | | | | BAA-1386 | | | | | |
| 1803 | GCA_000473915.1 | 1219013 | 43767 | Rhodococcus equi NBRC 101255 = C 7 | C 7 | Actinobacteria | APJC01000016.1 | 2 | ERN46351.1 | 17740 |
| 1804 | GCA_001467645.1 | 45065 | 45065 | Legionella geestiana | ATCC 49504 | Proteobacteria | LNYC01000024.1 | 2 | KTD01946.1 | 17742 |
| 1805 | GCA_000967915.1 | 61635 | 61635 | Acholeplasma brassicae | O502 | Tenericutes | FO681348.1 | 2 | CCV65562.1 | 17744 |
| 1806 | GCA_001038235.1 | 61647 | 61647 | Pluralibacter gergoviae | DS82E24 | Proteobacteria | LDZG01000031.1 | 2 | KMK02865.1 | 17746 |
| 1807 | GCA_000568815.1 | 1150621 | 66821 | Sulfurospirillum multivorans DSM 12446 | DSM 12446 | Proteobacteria | CP007201.1 | 2 | AHJ12534.1 | 17748 |
| 1808 | GCA_001509475.1 | 68262 | 68262 | Streptomyces regalis | NRRL 3151 | Actinobacteria | LLZG01000047.1 | 2 | KUL42778.1 | 17750 |
| 1809 | GCA_000257785.1 | 1095730 | 76860 | Streptococcus constellatus subsp. | SK53 | Firmicutes | AICQ01000008.1 | 2 | EID21980.1 | 17752 |
| | | | | constellatus SK53 | | | | | | |
| 1810 | GCA_000474075.1 | 1095730 | 76860 | Streptococcus constellatus subsp. | SK53 | Firmicutes | BASU01000001.1 | 2 | GAD37767.1 | 17754 |
| | | | | constellatus SK53 | | | | | | |
| 1811 | GCA_001434855.1 | 1423788 | 83526 | Lactobacillus paralimentarius DSM 19674 | DSM 19674 | Firmicutes | AZDY01000037.1 | 2 | KRK83038.1 | 17756 |
| 1812 | GCA_000186185.1 | 562971 | 85698 | Achromobacter xylosoxidans C54 | C54 | Proteobacteria | GL636034.1 | 2 | EFV85442.1 | 17758 |
| 1813 | GCA_000364225.1 | 1235802 | 97253 | Eubacterium plexicaudatum ASF492 | ASF492 | Firmicutes | KB822499.1 | 2 | EMZ26178.1 | 17760 |
| 1814 | GCA_000317595.1 | 698759 | 103232 | Streptomyces ipomoeae 91-03 | 91-03 | Actinobacteria | AEJC01000486.1 | 2 | EKX62822.1 | 17762 |
| 1815 | GCA_000017145.1 | 366394 | 110321 | Sinorhizobium medicae WSM419 | WSM419 | Proteobacteria | CP000740.1 | 2 | ABR64200.1 | 17764 |
| 1816 | GCA_001188555.1 | 121719 | 121719 | Pannonibacter phragmitetus | CGMCC9175 | Proteobacteria | LGSQ01000007.1 | 2 | KND18807.1 | 17766 |
| 1817 | GCA_000092505.1 | 762051 | 136609 | Leuconostoc kimchii IMSNU 11154 | IMSNU 11154 | Firmicutes | CP001758.1 | 2 | ADG41466.1 | 17768 |
| 1818 | GCA_000156215.1 | 445975 | 147206 | Collinsella stercoris DSM 13279 | DSM 13279 | Actinobacteria | DS995474.1 | 2 | EEA91362.1 | 17770 |
| 1819 | GCA_000235505.1 | 742737 | 154046 | Hungatella hathewayi WAL-18680 | WAL-18680 | Firmicutes | JH379027.1 | 2 | EHI61152.1 | 17772 |
| 1820 | GCA_000178255.1 | 702450 | 154288 | Turicibacter sanguinis PC909 | PC909 | Firmicutes | ADMN01000123.1 | 2 | EFF62517.1 | 17774 |
| 1821 | GCA_001465255.1 | 162209 | 162209 | Paenibacillus naphthalenovorans | 32O-Y | Firmicutes | CP013652.1 | 2 | ALS25374.1 | 17776 |
| 1822 | GCA_000767835.1 | 176090 | 176090 | Streptococcus sinensis | HKU4 | Firmicutes | JPEN01000062.1 | 2 | KGM37271.1 | 17778 |
| 1823 | GCA_000568075.1 | 314330 | 314330 | Enterobacter sp. DC1 | DC1 | Proteobacteria | AZUA01000002.1 | 2 | EWG70341.1 | 17780 |
| 1824 | GCA_001050135.1 | 320787 | 320787 | Cyclobacterium amurskyense | KCTC 12363 | Bacteroidetes | CP012040.1 | 2 | AKP49793.1 | 17782 |
| 1825 | GCA_000964065.1 | 332411 | 332411 | Aquitalea magnusonii | SM6 | Proteobacteria | JZRC01000020.1 | 2 | KJV29339.1 | 17784 |
| 1826 | GCA_000264295.1 | 1163407 | 347483 | Rhodanobacter spathiphylli B39 | B39 | Proteobacteria | AJXT01000055.1 | 2 | EIL89963.1 | 17786 |
| 1827 | GCA_000737685.1 | 362418 | 362418 | Flavobacterium reichenbachii | LMG 25512 | Bacteroidetes | JPRL01000001.1 | 2 | KFF05553.1 | 17788 |
| 1828 | GCA_001405815.1 | 388408 | 388408 | Labrenzia alexandrii | CECT 5112 | Proteobacteria | CXWD01000013.1 | 2 | CTQ72905.1 | 17790 |
| 1829 | GCA_001444505.1 | 554343 | 554343 | Psychrobacter piscatorii | LQ58 | Proteobacteria | LNDJ01000085.1 | 2 | KRU21991.1 | 17792 |
| 1830 | GCA_000956505.1 | 582680 | 582680 | Microbacterium azadirachtae | ARN176 | Actinobacteria | JYIX01000037.1 | 2 | KJL32259.1 | 17794 |
| 1831 | GCA_000245715.1 | 1050222 | 1050222 | Paenibacillus sp. Aloe-11 | Aloe-11 | Firmicutes | JH601082.1 | 2 | EHS54840.1 | 17796 |
| 1832 | GCA_000282055.1 | 1144313 | 1144313 | Flavobacterium sp. CF136 | CF136 | Bacteroidetes | AKJZ01000027.1 | 2 | EJL63880.1 | 17798 |
| 1833 | GCA_000282675.1 | 1144320 | 1144320 | Pantoea sp. GM01 | GM01 | Proteobacteria | AKIU01000064.1 | 2 | EJL84705.1 | 17800 |
| 1834 | GCA_000260835.1 | 946077 | 1165090 | Imtechella halotolerans K1 | K1 | Bacteroidetes | AJJU01000017.1 | 2 | EID73851.1 | 17802 |
| 1835 | GCA_001532035.1 | 1207504 | 1207504 | Burkholderia pseudomultivorans | MSMB368WGS | Proteobacteria | LPJR01000094.1 | 2 | KWF17574.1 | 17804 |
| 1836 | GCA_000466565.1 | 1226324 | 1226324 | Blautia sp. KLE 1732 | KLE 1732 | Firmicutes | KE993386.1 | 2 | ERI96811.1 | 17806 |
| 1837 | GCA_000403295.2 | 1235792 | 1235792 | Lachnospiraceae bacterium M18-1 | M18-1 | Firmicutes | KE159609.1 | 2 | EOS38323.1 | 17808 |
| 1838 | GCA_000403335.2 | 1235793 | 1235793 | Lachnospiraceae bacterium COE1 | COE1 | Firmicutes | KE159632.1 | 2 | EOS41468.1 | 17810 |
| 1839 | GCA_000438215.1 | 1263066 | 1263066 | Clostridium clostridioforme CAG:511 | | Firmicutes | FR903693.1 | 2 | CDF26098.1 | 17812 |
| 1840 | GCA_000768755.1 | 1537504 | 1537504 | Porphyromonas sp. COT-108 OH1349 | COT-108 | Bacteroidetes | JRAH01000034.1 | 2 | KGN67552.1 | 17814 |
| | | | | | OH1349 | | | | | |
| 1841 | GCA_001508375.1 | 1635262 | 1635262 | Desulfotomaculum sp. 46_296 | | Firmicutes | LGFZ01000024.1 | 2 | KUK53685.1 | 17816 |
| 1842 | GCA_000988325.1 | 1643451 | 1643451 | Sphingobacterium sp. Ag1 | Ag1 | Bacteroidetes | LBGU01000044.1 | 2 | KKO89442.1 | 17818 |
| 1843 | GCA_001187905.1 | 1681195 | 1681195 | Pantoea sp. RIT-PI-b | RIT-PI-b | Proteobacteria | LGIS01000026.1 | 2 | KNC08320.1 | 17820 |
| 1844 | GCA_001422575.1 | 1736224 | 1736224 | Serratia sp. Leaf51 | Leaf51 | Proteobacteria | LMU01000057.1 | 2 | KQN48064.1 | 17822 |
| 1845 | GCA_000979175.1 | 2209 | 2209 | Methanosarcina mazei | 1.H.A.1A.3 | Euryarchaeota | JJQH01000125.1 | 2 | KKH38512.1 | 17824 |
| 1846 | GCA_000979195.1 | 2209 | 2209 | Methanosarcina mazei | 1.H.A.2.3 | Euryarchaeota | JJQM01000126.1 | 2 | KKH53104.1 | 17826 |
| 1847 | GCA_000979205.1 | 2209 | 2209 | Methanosarcina mazei | 1.H.A.2.7 | Euryarchaeota | JJQ001000047.1 | 2 | KKH68857.1 | 17828 |
| 1848 | GCA_000979255.1 | 2209 | 2209 | Methanosarcina mazei | 1.H.M.1A.2 | Euryarchaeota | JJQS01000129.1 | 2 | KKH71868.1 | 17830 |
| 1849 | GCA_000979275.1 | 2209 | 2209 | Methanosarcina mazei | 1.H.M.2.2 | Euryarchaeota | JJQV01000133.1 | 2 | KKH80640.1 | 17832 |
| 1850 | GCA_000979335.1 | 2209 | 2209 | Methanosarcina mazei | 2.F.A.2.3 | Euryarchaeota | JJOR01000174.1 | 2 | KKF98496.1 | 17834 |
| 1851 | GCA_000979925.1 | 2209 | 2209 | Methanosarcina mazei | 1.H.A.1A.6 | Euryarchaeota | JJQJ01000061.1 | 2 | KKH51286.1 | 17836 |
| 1852 | GCA_000980005.1 | 2209 | 2209 | Methanosarcina mazei | 1.H.A.2.8 | Euryarchaeota | JJQP01000201.1 | 2 | KKH64073.1 | 17838 |
| 1853 | GCA_000980055.1 | 2209 | 2209 | Methanosarcina mazei | 1.H.M.1A.1 | Euryarchaeota | JJQR01000061.1 | 2 | KKH76320.1 | 17840 |
| 1854 | GCA_000980075.1 | 2209 | 2209 | Methanosarcina mazei | 1.H.M.2.3 | Euryarchaeota | JJQW01000059.1 | 2 | KKH88481.1 | 17842 |
| 1855 | GCA_000980085.1 | 2209 | 2209 | Methanosarcina mazei | 1.H.M.1A.3 | Euryarchaeota | JJQT01000187.1 | 2 | KKH75119.1 | 17844 |
| 1856 | GCA_000980105.1 | 2209 | 2209 | Methanosarcina mazei | 1.H.M.2.4 | Euryarchaeota | JJQX01000190.1 | 2 | KKH91069.1 | 17846 |
| 1857 | GCA_000980135.1 | 2209 | 2209 | Methanosarcina mazei | 1.H.T.2.1 | Euryarchaeota | JJQZ01000005.1 | 2 | KKI00020.1 | 17848 |
| 1858 | GCA_000980155.1 | 2209 | 2209 | Methanosarcina mazei | 1.H.T.2.3 | Euryarchaeota | JJRA01000002.1 | 2 | KKI06936.1 | 17850 |
| 1859 | GCA_000980175.1 | 2209 | 2209 | Methanosarcina mazei | 1.H.T.2.5 | Euryarchaeota | JJRB01000041.1 | 2 | KKI04817.1 | 17852 |
| 1860 | GCA_000465655.1 | 1390372 | 1390 | Bacillus amyloliquefaciens EGD-AQ14 | EGD-AQ14 | Firmicutes | AVQH01000001.1 | 2 | ERH59124.1 | 17854 |
| 1861 | GCA_000092605.1 | 75379 | 926 | Thiomonas intermedia K12 | K12 | Proteobacteria | CP002021.1 | 3 | ADG31357.1 | 17856 |
| 1862 | GCA_001263815.1 | 837 | 837 | Porphyromonas gingivalis | A7436 | Bacteroidetes | CP011995.1 | 3 | AKV63928.1 | 17859 |
| 1863 | GCA_000436435.1 | 1263050 | 1263050 | Bacteroides ovatus CAG:22 | | Bacteroidetes | FR885648.1 | 3 | CDB59852.1 | 17862 |
| 1864 | GCA_000154245.1 | 411489 | 411489 | Clostridium sp. L2-50 | L2-50 | Firmicutes | DS480312.1 | 3 | ED056710.1 | 17865 |
| 1865 | GCA_000768935.1 | 1515615 | 1515615 | Porphyromonas sp. COT-290 OH860 | COT-290 | Bacteroidetes | JRAR01000072.1 | 3 | KGN82593.1 | 17868 |
| | | | | | OH860 | | | | | |
| 1866 | GCA_001183745.1 | 438 | 438 | Acetobacter pasteurianus | Ab 3 | Proteobacteria | CP012111.1 | 3 | AKR49340.1 | 17871 |
| 1867 | GCA_000496775.1 | 1399146 | 550 | Enterobacter cloacae S611 | S611 | Proteobacteria | AXOM01000048.1 | 3 | ESS57310.1 | 17874 |
| 1868 | GCA_000007445.1 | 199310 | 562 | Escherichia coli CFT073 | CFT073 | Proteobacteria | AE014075.1 | 3 | AAN80859.1 | 17877 |
| 1869 | GCA_000164295.1 | 749528 | 562 | Escherichia coli MS 45-1 | MS 45-1 | Proteobacteria | GG772205.1 | 3 | EFJ93881.1 | 17880 |
| 1870 | GCA_000164415.1 | 749536 | 562 | Escherichia coli MS 110-3 | MS 110-3 | Proteobacteria | GG773033.1 | 3 | EFU46133.1 | 17883 |
| 1871 | GCA_000164435.1 | 749541 | 562 | Escherichia coli MS 153-1 | MS 153-1 | Proteobacteria | GG773212.1 | 3 | EFU49595.1 | 17886 |
| 1872 | GCA_000164535.1 | 749550 | 562 | Escherichia coli MS 200-1 | MS 200-1 | Proteobacteria | GG773863.1 | 3 | EFJ59502.1 | 17889 |
| 1873 | GCA_000164595.1 | 749530 | 562 | Escherichia coli MS 60-1 | MS 60-1 | Proteobacteria | GG774357.1 | 3 | EGB82665.1 | 17892 |
| 1874 | GCA_000179795.1 | 753642 | 562 | Escherichia coli NC101 | NC101 | Proteobacteria | AEFA01000016.1 | 3 | EFM52806.1 | 17895 |
| 1875 | GCA_000190915.1 | 656390 | 562 | Escherichia coli H263 | H263 | Proteobacteria | GL871979.1 | 3 | EGB52118.1 | 17898 |
| 1876 | GCA_000233875.1 | 885276 | 562 | Escherichia coli str. ‘clone D i2’ | clone D i2 | Proteobacteria | CP002211.1 | 3 | AER84780.1 | 17901 |
| 1877 | GCA_000233895.1 | 885275 | 562 | Escherichia coli str. ‘clone D i14’ | clone D i14 | Proteobacteria | CP002212.1 | 3 | AER89699.1 | 17904 |
| 1878 | GCA_000241975.1 | 656398 | 562 | Escherichia coli H397 | H397 | Proteobacteria | JH590471.1 | 3 | EH002519.1 | 17907 |
| 1879 | GCA_000295775.2 | 1206108 | 562 | Escherichia coli J96 | J96 | Proteobacteria | ALIN02000009.1 | 3 | ELL42848.1 | 17910 |
| 1880 | GCA_000326165.1 | 1169374 | 562 | Escherichia coli KTE106 | KTE106 | Proteobacteria | ANXN01000036.1 | 3 | ELI13536.1 | 17913 |
| 1881 | GCA_000326225.1 | 1169381 | 562 | Escherichia coli KTE124 | KTE124 | Proteobacteria | ANXU01000045.1 | 3 | ELI43906.1 | 17916 |
| 1882 | GCA_000326285.1 | 1169385 | 562 | Escherichia coli KTE131 | KTE131 | Proteobacteria | ANXY01000031.1 | 3 | ELI67626.1 | 17919 |
| 1883 | GCA_000326305.1 | 1169386 | 562 | Escherichia coli KTE133 | KTE133 | Proteobacteria | ANXZ01000016.1 | 3 | ELI72589.1 | 17922 |
| 1884 | GCA_000326325.1 | 1169387 | 562 | Escherichia coli KTE137 | KTE137 | Proteobacteria | ANYA01000031.1 | 3 | ELI75960.1 | 17925 |
| 1885 | GCA_000326385.1 | 1169393 | 562 | Escherichia coli KTE153 | KTE153 | Proteobacteria | ANYG01000025.1 | 3 | EU03561.1 | 17928 |
| 1886 | GCA_000326405.1 | 1169395 | 562 | Escherichia coli KTE160 | KTE160 | Proteobacteria | ANYI01000026.1 | 3 | EU14334.1 | 17931 |
| 1887 | GCA_000326605.1 | 1169400 | 562 | Escherichia coli KTE174 | KTE174 | Proteobacteria | ANYN01000054.1 | 3 | EU38405.1 | 17934 |
| 1888 | GCA_000326625.1 | 1169366 | 562 | Escherichia coli KTE88 | KTE88 | Proteobacteria | ANYV01000037.1 | 3 | EU71313.1 | 17937 |
| 1889 | GCA_000326705.1 | 1169390 | 562 | Escherichia coli KTE145 | KTE145 | Proteobacteria | ANYD01000026.1 | 3 | ELI89432.1 | 17940 |
| 1890 | GCA_000326905.1 | 1169398 | 562 | Escherichia coli KTE167 | KTE167 | Proteobacteria | KB731668.1 | 3 | EU28710.1 | 17943 |
| 1891 | GCA_000350745.1 | 1169334 | 562 | Escherichia coli KTE15 | KTE15 | Proteobacteria | KB732276.1 | 3 | ELC30031.1 | 17946 |
| 1892 | GCA_000350985.1 | 1181735 | 562 | Escherichia coli KTE189 | KTE189 | Proteobacteria | KB732437.1 | 3 | ELC84584.1 | 17949 |
| 1893 | GCA_000351005.1 | 1181737 | 562 | Escherichia coli KTE191 | KTE191 | Proteobacteria | KB732446.1 | 3 | ELC89744.1 | 17952 |
| 1894 | GCA_000351045.1 | 1181747 | 562 | Escherichia coli KTE201 | KTE201 | Proteobacteria | KB732482.1 | 3 | ELC99568.1 | 17955 |
| 1895 | GCA_000351105.1 | 1181752 | 562 | Escherichia coli KTE206 | KTE206 | Proteobacteria | KB732494.1 | 3 | ELD14727.1 | 17958 |
| 1896 | GCA_000351245.1 | 1181766 | 562 | Escherichia coli KTE220 | KTE220 | Proteobacteria | KB733367.1 | 3 | ELD50500.1 | 17961 |
| 1897 | GCA_000351485.1 | 1182660 | 562 | Escherichia coli KTE53 | KTE53 | Proteobacteria | KB732687.1 | 3 | ELE05785.1 | 17964 |
| 1898 | GCA_000351545.1 | 1182664 | 562 | Escherichia coli KTE57 | KTE57 | Proteobacteria | KB732714.1 | 3 | ELE19657.1 | 17967 |
| 1899 | GCA_000351565.1 | 1182665 | 562 | Escherichia coli KTE58 | KTE58 | Proteobacteria | KB732739.1 | 3 | ELE23560.1 | 17970 |
| 1900 | GCA_000351665.1 | 1182679 | 562 | Escherichia coli KTE72 | KTE72 | Proteobacteria | KB733397.1 | 3 | ELE50401.1 | 17973 |
| 1901 | GCA_000351805.1 | 1182691 | 562 | Escherichia coli KTE86 | KTE86 | Proteobacteria | KB732872.1 | 3 | ELE80392.1 | 17976 |
| 1902 | GCA_000351825.1 | 1182692 | 562 | Escherichia coli KTE87 | KTE87 | Proteobacteria | KB732881.1 | 3 | ELE90662.1 | 17979 |
| 1903 | GCA_000351845.1 | 1182696 | 562 | Escherichia coli KTE93 | KTE93 | Proteobacteria | KB732897.1 | 3 | ELE91478.1 | 17982 |
| 1904 | GCA_000352085.1 | 1169328 | 562 | Escherichia coli KTE8 | KTE8 | Proteobacteria | KB732984.1 | 3 | ELF50405.1 | 17985 |
| 1905 | GCA_000352265.1 | 1169341 | 562 | Escherichia coli KTE22 | KTE22 | Proteobacteria | KB733023.1 | 3 | ELF92090.1 | 17988 |
| 1906 | GCA_000352365.1 | 1182666 | 562 | Escherichia coli KTE59 | KTE59 | Proteobacteria | KB733042.1 | 3 | ELG14338.1 | 17991 |
| 1907 | GCA_000352385.1 | 1182670 | 562 | Escherichia coli KTE63 | KTE63 | Proteobacteria | KB733049.1 | 3 | ELG17003.1 | 17994 |
| 1908 | GCA_000352405.1 | 1182672 | 562 | Escherichia coli KTE65 | KTE65 | Proteobacteria | KB733066.1 | 3 | ELG24591.1 | 17997 |
| 1909 | GCA_000352545.1 | 1182709 | 562 | Escherichia coli KTE118 | KTE118 | Proteobacteria | KB733111.1 | 3 | ELG54538.1 | 18000 |
| 1910 | GCA_000352565.1 | 1182712 | 562 | Escherichia coli KTE123 | KTE123 | Proteobacteria | KB733071.1 | 3 | ELG58278.1 | 18003 |
| 1911 | GCA_000352905.1 | 1181729 | 562 | Escherichia coli KTE183 | KTE183 | Proteobacteria | KB733173.1 | 3 | ELH44356.1 | 18006 |
| 1912 | GCA_000353005.1 | 1181753 | 562 | Escherichia coli KTE207 | KTE207 | Proteobacteria | KB733196.1 | 3 | ELH62529.1 | 18009 |
| 1913 | GCA_000353025.1 | 1181755 | 562 | Escherichia coli KTE209 | KTE209 | Proteobacteria | KB733361.1 | 3 | ELH59268.1 | 18012 |
| 1914 | GCA_000353065.1 | 1181761 | 562 | Escherichia coli KTE215 | KTE215 | Proteobacteria | KB733208.1 | 3 | ELH80077.1 | 18015 |
| 1915 | GCA_000353105.1 | 1181764 | 562 | Escherichia coli KTE218 | KTE218 | Proteobacteria | KB733215.1 | 3 | ELH85630.1 | 18018 |
| 1916 | GCA_000353125.1 | 1181769 | 562 | Escherichia coli KTE223 | KTE223 | Proteobacteria | KB733352.1 | 3 | ELH88471.1 | 18021 |
| 1917 | GCA_000353185.1 | 1169372 | 562 | Escherichia coli KTE104 | KTE104 | Proteobacteria | KB733272.1 | 3 | ELI08747.1 | 18024 |
| 1918 | GCA_000401755.1 | 1322345 | 562 | Escherichia coli ATCC 25922 | ATCC 25922 | Proteobacteria | ASHD01000045.1 | 3 | EOR52540.1 | 18027 |
| 1919 | GCA_000408125.1 | 1181741 | 562 | Escherichia coli KTE195 | KTE195 | Proteobacteria | KE136905.1 | 3 | EOV07668.1 | 18030 |
| 1920 | GCA_000408285.1 | 1181772 | 562 | Escherichia coli KTE226 | KTE226 | Proteobacteria | KE136996.1 | 3 | EOX10652.1 | 18033 |
| 1921 | GCA_000408505.1 | 1182693 | 562 | Escherichia coli KTE89 | KTE89 | Proteobacteria | KE137142.1 | 3 | EOV94186.1 | 18036 |
| 1922 | GCA_000456005.1 | 1280956 | 562 | Escherichia coli HVH 1 (4-6876161) | HVH 1 (4-6876161) | Proteobacteria | KE698776.1 | 3 | EQN11053.1 | 18039 |
| 1923 | GCA_000456065.1 | 1280959 | 562 | Escherichia coli HVH 4 (4-7276109) | HVH 4 (4-7276109) | Proteobacteria | KE698831.1 | 3 | EQN19685.1 | 18042 |
| 1924 | GCA_000456125.1 | 1280962 | 562 | Escherichia coli HVH 7 (4-7315031) | HVH 7 (4-7315031) | Proteobacteria | KE698877.1 | 3 | EQN35019.1 | 18045 |
| 1925 | GCA_000456205.1 | 1280967 | 562 | Escherichia coli HVH 16 (4-7649002) | HVH 16 (4-7649002) | Proteobacteria | KE698922.1 | 3 | EQN48989.1 | 18048 |
| 1926 | GCA_000456285.1 | 1280971 | 562 | Escherichia coli HVH 20 (4-5865042) | HVH 20 (4-5865042) | Proteobacteria | KE699006.1 | 3 | EQN62516.1 | 18051 |
| 1927 | GCA_000456305.1 | 1280972 | 562 | Escherichia coli HVH 21 (4-4517873) | HVH 21 (4-4517873) | Proteobacteria | KE699022.1 | 3 | EQN79331.1 | 18054 |
| 1928 | GCA_000456385.1 | 1280977 | 562 | Escherichia coli HVH 26 (4-5703913) | HVH 26 (4-5703913) | Proteobacteria | KE699069.1 | 3 | EQN86135.1 | 18057 |
| 1929 | GCA_000456405.1 | 1280978 | 562 | Escherichia coli HVH 27 (4-7449267) | HVH 27 (4-7449267) | Proteobacteria | KE699090.1 | 3 | EQN96595.1 | 18060 |
| 1930 | GCA_000456425.1 | 1280979 | 562 | Escherichia coli HVH 28 (4-0907367) | HVH 28 (4-0907367) | Proteobacteria | KE699097.1 | 3 | EQO12273.1 | 18063 |
| 1931 | GCA_000456465.1 | 1280981 | 562 | Escherichia coli HVH 30 (4-2661829) | HVH 30 (4-2661829) | Proteobacteria | KE699120.1 | 3 | EQO16638.1 | 18066 |
| 1932 | GCA_000456485.1 | 1280982 | 562 | Escherichia coli HVH 31 (4-2602156) | HVH 31 (4-2602156) | Proteobacteria | KE699127.1 | 3 | EQO18515.1 | 18069 |
| 1933 | GCA_000456545.1 | 1280985 | 562 | Escherichia coli HVH 35 (4-2962667) | HVH 35 (4-2962667) | Proteobacteria | KE699157.1 | 3 | EQO32111.1 | 18072 |
| 1934 | GCA_000456565.1 | 1280987 | 562 | Escherichia coli HVH 37 (4-2773848) | HVH 37 (4-2773848) | Proteobacteria | KE699170.1 | 3 | EQO40647.1 | 18075 |
| 1935 | GCA_000456625.1 | 1280990 | 562 | Escherichia coli HVH 40 (4-1219782) | HVH 40 (4-1219782) | Proteobacteria | KE699214.1 | 3 | EQO47387.1 | 18078 |
| 1936 | GCA_000456765.1 | 1280997 | 562 | Escherichia coli HVH 48 (4-2658593) | HVH 48 (4-2658593) | Proteobacteria | KE699294.1 | 3 | EQO84680.1 | 18081 |
| 1937 | GCA_000456785.1 | 1280999 | 562 | Escherichia coli HVH 51 (4-2172526) | HVH 51 (4-2172526) | Proteobacteria | KE699304.1 | 3 | EQO93036.1 | 18084 |
| 1938 | GCA_000456825.1 | 1281002 | 562 | Escherichia coli HVH 55 (4-2646161) | HVH 55 (4-2646161) | Proteobacteria | KE699321.1 | 3 | EQP00513.1 | 18087 |
| 1939 | GCA_000456845.1 | 1281003 | 562 | Escherichia coli HVH 56 (4-2153033) | HVH 56 (4-2153033) | Proteobacteria | KE699325.1 | 3 | EQP09250.1 | 18090 |
| 1940 | GCA_000456865.1 | 1281004 | 562 | Escherichia coli HVH 58 (4-2839709) | HVH 58 (4-2839709) | Proteobacteria | KE699335.1 | 3 | EQP11538.1 | 18093 |
| 1941 | GCA_000456905.1 | 1281006 | 562 | Escherichia coli HVH 61 (4-2736020) | HVH 61 (4-2736020) | Proteobacteria | KE699360.1 | 3 | EQP21163.1 | 18096 |
| 1942 | GCA_000456965.1 | 1281009 | 562 | Escherichia coli HVH 68 (4-0888028) | HVH 68 (4-0888028) | Proteobacteria | KE699393.1 | 3 | EQP38130.1 | 18099 |
| 1943 | GCA_000457005.1 | 1281011 | 562 | Escherichia coli HVH 70 (4-2963531) | HVH 70 (4-2963531) | Proteobacteria | KE699425.1 | 3 | EQP55415.1 | 18102 |
| 1944 | GCA_000457045.1 | 1281013 | 562 | Escherichia coli HVH 74 (4-1034782) | HVH 74 (4-1034782) | Proteobacteria | KE699452.1 | 3 | EQP53333.1 | 18105 |
| 1945 | GCA_000457065.1 | 1281014 | 562 | Escherichia coli HVH 76 (4-2538717) | HVH 76 (4-2538717) | Proteobacteria | KE699459.1 | 3 | EQP61514.1 | 18108 |
| 1946 | GCA_000457085.1 | 1281015 | 562 | Escherichia coli HVH 77 (4-2605759) | HVH 77 (4-2605759) | Proteobacteria | KE699520.1 | 3 | EQP73530.1 | 18111 |
| 1947 | GCA_000457105.1 | 1281016 | 562 | Escherichia coli HVH 78 (4-2735946) | HVH 78 (4-2735946) | Proteobacteria | KE699531.1 | 3 | EQP70429.1 | 18114 |
| 1948 | GCA_000457265.1 | 1281026 | 562 | Escherichia coli HVH 89 (4-5885604) | HVH 89 (4-5885604) | Proteobacteria | KE699634.1 | 3 | EQQ06480.1 | 18117 |
| 1949 | GCA_000457325.1 | 1281029 | 562 | Escherichia coli HVH 92 (4-5930790) | HVH 92 (4-5930790) | Proteobacteria | KE705250.1 | 3 | EQQ25252.1 | 18120 |
| 1950 | GCA_000457345.1 | 1281031 | 562 | Escherichia coli HVH 95 (4-6074464) | HVH 95 (4-6074464) | Proteobacteria | KE699681.1 | 3 | EQQ27933.1 | 18123 |
| 1951 | GCA_000457385.1 | 1281032 | 562 | Escherichia coli HVH 96 (4-5934869) | HVH 96 (4-5934869) | Proteobacteria | KE699694.1 | 3 | EQQ41432.1 | 18126 |
| 1952 | GCA_000457405.1 | 1281036 | 562 | Escherichia coli HVH 100 (4-2850729) | HVH 100 (4-2850729) | Proteobacteria | KE699702.1 | 3 | EQQ33647.1 | 18129 |
| 1953 | GCA_000457435.1 | 1281039 | 562 | Escherichia coli HVH 103 (4-5904188) | HVH 103 (4-5904188) | Proteobacteria | KE699715.1 | 3 | EQQ53134.1 | 18132 |
| 1954 | GCA_000457515.1 | 1281045 | 562 | Escherichia coli HVH 109 (4-6977162) | HVH 109 (4-6977162) | Proteobacteria | KE699785.1 | 3 | EQQ74422.1 | 18135 |
| 1955 | GCA_000457555.1 | 1281047 | 562 | Escherichia coli HVH 111 (4-7039018) | HVH 111 (4-7039018) | Proteobacteria | KE699806.1 | 3 | EQQ75981.1 | 18138 |
| 1956 | GCA_000457575.1 | 1281048 | 562 | Escherichia coli HVH 112 (4-5987253) | HVH 112 (4-5987253) | Proteobacteria | KE699821.1 | 3 | EQQ88668.1 | 18141 |
| 1957 | GCA_000457615.1 | 1281050 | 562 | Escherichia coli HVH 114 (4-7037740) | HVH 114 (4-7037740) | Proteobacteria | KE699847.1 | 3 | EQQ90545.1 | 18144 |
| 1958 | GCA_000457675.1 | 1281053 | 562 | Escherichia coli HVH 116 (4-6879942) | HVH 116 (4-6879942) | Proteobacteria | KE699890.1 | 3 | EQR07410.1 | 18147 |
| 1959 | GCA_000457695.1 | 1281054 | 562 | Escherichia coli HVH 117 (4-6857191) | HVH 117(4-6857191) | Proteobacteria | KE699912.1 | 3 | EQR15138.1 | 18150 |
| 1960 | GCA_000457715.1 | 1281055 | 562 | Escherichia coli HVH 118 (4-7345399) | HVH 118 (4-7345399) | Proteobacteria | KE699926.1 | 3 | EQR19371.1 | 18153 |
| 1961 | GCA_000457755.1 | 1281057 | 562 | Escherichia coli HVH 120 (4-6978681) | HVH 120 (4-6978681) | Proteobacteria | KE699947.1 | 3 | EQR28930.1 | 18156 |
| 1962 | GCA_000457915.1 | 1281065 | 562 | Escherichia coli HVH 132 (4-6876862) | HVH 132 (4-6876862) | Proteobacteria | KE700080.1 | 3 | EQR67054.1 | 18159 |
| 1963 | GCA_000457995.1 | 1281070 | 562 | Escherichia coli HVH 137 (4-2124971) | HVH 137(4-2124971) | Proteobacteria | KE700110.1 | 3 | EQR88164.1 | 18162 |
| 1964 | GCA_000458015.1 | 1281071 | 562 | Escherichia coli HVH 138 (4-6066704) | HVH 138 (4-6066704) | Proteobacteria | KE700120.1 | 3 | EQR94358.1 | 18165 |
| 1965 | GCA_000458095.1 | 1281075 | 562 | Escherichia coli HVH 142 (4-5627451) | HVH 142 (4-5627451) | Proteobacteria | KE700203.1 | 3 | EQS19450.1 | 18168 |
| 1966 | GCA_000458115.1 | 1281076 | 562 | Escherichia coli HVH 143 (4-5674999) | HVH 143 (4-5674999) | Proteobacteria | KE700210.1 | 3 | EQS13124.1 | 18171 |
| 1967 | GCA_000458135.1 | 1281077 | 562 | Escherichia coli HVH 144 (4-4451937) | HVH 144 (4-4451937) | Proteobacteria | KE700223.1 | 3 | EQS20959.1 | 18174 |
| 1968 | GCA_000458215.1 | 1281082 | 562 | Escherichia coli HVH 149 (4-4451880) | HVH 149 (4-4451880) | Proteobacteria | KE700251.1 | 3 | EQS40214.1 | 18177 |
| 1969 | GCA_000458355.1 | 1281090 | 562 | Escherichia coli HVH 157 (4-3406229) | HVH 157 (4-3406229) | Proteobacteria | KE700382.1 | 3 | ERA75001.1 | 18180 |
| 1970 | GCA_000458375.1 | 1281091 | 562 | Escherichia coli HVH 158 (4-3224287) | HVH 158 (4-3224287) | Proteobacteria | KE700388.1 | 3 | EQS62808.1 | 18183 |
| 1971 | GCA_000458395.1 | 1281092 | 562 | Escherichia coli HVH 159 (4-5818141) | HVH 159 (4-5818141) | Proteobacteria | KE700400.1 | 3 | ERA80457.1 | 18186 |
| 1972 | GCA_000458415.1 | 1281093 | 562 | Escherichia coli HVH 160 (4-5695937) | HVH 160 (4-5695937) | Proteobacteria | KE700412.1 | 3 | ERA86693.1 | 18189 |
| 1973 | GCA_000458435.1 | 1281094 | 562 | Escherichia coli HVH 161 (4-3119890) | HVH 161 (4-3119890) | Proteobacteria | KE700429.1 | 3 | EQS67056.1 | 18192 |
| 1974 | GCA_000458535.1 | 1281099 | 562 | Escherichia coli HVH 169 (4-1075578) | HVH 169 (4-1075578) | Proteobacteria | KE700489.1 | 3 | EQS96897.1 | 18195 |
| 1975 | GCA_000458575.1 | 1281101 | 562 | Escherichia coli HVH 171 (4-3191958) | HVH 171 (4-3191958) | Proteobacteria | KE700518.1 | 3 | EQT00426.1 | 18198 |
| 1976 | GCA_000458605.1 | 1281102 | 562 | Escherichia coli HVH 172 (4-3248542) | HVH 172 (4-3248542) | Proteobacteria | KE700534.1 | 3 | EQT14974.1 | 18201 |
| 1977 | GCA_000458645.1 | 1281104 | 562 | Escherichia coli HVH 175 (4-3405184) | HVH 175 (4-3405184) | Proteobacteria | KE700564.1 | 3 | EQT25531.1 | 18204 |
| 1978 | GCA_000458745.1 | 1281111 | 562 | Escherichia coli HVH 184 (4-3343286) | HVH 184 (4-3343286) | Proteobacteria | KE700641.1 | 3 | EQT45492.1 | 18207 |
| 1979 | GCA_000458765.1 | 1281112 | 562 | Escherichia coli HVH 185 (4-2876639) | HVH 185 (4-2876639) | Proteobacteria | KE700651.1 | 3 | EQT50681.1 | 18210 |
| 1980 | GCA_000458895.1 | 1281119 | 562 | Escherichia coli HVH 192 (4-3054470) | HVH 192 (4-3054470) | Proteobacteria | KE700778.1 | 3 | EQT83221.1 | 18213 |
| 1981 | GCA_000458935.1 | 1281121 | 562 | Escherichia coli HVH 194 (4-2356805) | HVH 194 (4-2356805) | Proteobacteria | KE700789.1 | 3 | EQT95718.1 | 18216 |
| 1982 | GCA_000458995.1 | 1281124 | 562 | Escherichia coli HVH 197 (4-4466217) | HVH 197 (4-4466217) | Proteobacteria | KE700810.1 | 3 | EQU21869.1 | 18219 |
| 1983 | GCA_000459035.1 | 1281126 | 562 | Escherichia coli HVH 199 (4-5670322) | HVH 199 (4-5670322) | Proteobacteria | KE700830.1 | 3 | EQU12098.1 | 18222 |
| 1984 | GCA_000459255.1 | 1281137 | 562 | Escherichia coli HVH 210 (4-3042480) | HVH 210 (4-3042480) | Proteobacteria | KE700929.1 | 3 | ERA90778.1 | 18225 |
| 1985 | GCA_000459275.1 | 1281138 | 562 | Escherichia coli HVH 211 (4-3041891) | HVH 211 (4-3041891) | Proteobacteria | KE700944.1 | 3 | EQU70206.1 | 18228 |
| 1986 | GCA_000459295.1 | 1281139 | 562 | Escherichia coli HVH 212 (3-9305343) | HVH 212 (3-9305343) | Proteobacteria | KE700966.1 | 3 | EQU77946.1 | 18231 |
| 1987 | GCA_000459355.1 | 1281143 | 562 | Escherichia coli HVH 216 (4-3042952) | HVH 216 (4-3042952) | Proteobacteria | KE701004.1 | 3 | EQU95026.1 | 18234 |
| 1988 | GCA_000459395.1 | 1281145 | 562 | Escherichia coli HVH 218 (4-4500903) | HVH 218 (4-4500903) | Proteobacteria | KE701041.1 | 3 | EQV01575.1 | 18237 |
| 1989 | GCA_000459415.1 | 1281146 | 562 | Escherichia coli HVH 220 (4-5876842) | HVH 220 (4-5876842) | Proteobacteria | KE701064.1 | 3 | EQV09456.1 | 18240 |
| 1990 | GCA_000459515.1 | 1281151 | 562 | Escherichia coli HVH 227 (4-2277670) | HVH 227 (4-2277670) | Proteobacteria | KE701157.1 | 3 | EQV27904.1 | 18243 |
| 1991 | GCA_000459535.1 | 1281152 | 562 | Escherichia coli HVH 228 (4-7787030) | HVH 228 (4-7787030) | Proteobacteria | KE701167.1 | 3 | ERA94628.1 | 18246 |
| 1992 | GCA_000459595.1 | 1281155 | 562 | Escherichia coli KOEGE 10 (25a) | KOEGE 10 (25a) | Proteobacteria | KE701219.1 | 3 | ERB07224.1 | 18249 |
| 1993 | GCA_000459615.1 | 1281156 | 562 | Escherichia coli KOEGE 30 (63a) | KOEGE 30 (63a) | Proteobacteria | KE701229.1 | 3 | EQV33722.1 | 18252 |
| 1994 | GCA_000459755.1 | 1281163 | 562 | Escherichia coli KOEGE 58 (171a) | KOEGE 58 (171a) | Proteobacteria | KE701333.1 | 3 | EQV70972.1 | 18255 |
| 1995 | GCA_000459835.1 | 1281167 | 562 | Escherichia coli KOEGE 70 (185a) | KOEGE 70 (185a) | Proteobacteria | KE701373.1 | 3 | EQV84934.1 | 18258 |
| 1996 | GCA_000460055.1 | 1281178 | 562 | Escherichia coli UMEA 3053-1 | UMEA 3053-1 | Proteobacteria | KE701486.1 | 3 | EQW43215.1 | 18261 |
| 1997 | GCA_000460175.1 | 1281184 | 562 | Escherichia coli UMEA 3113-1 | UMEA 3113-1 | Proteobacteria | KE701574.1 | 3 | EQW70393.1 | 18264 |
| 1998 | GCA_000460215.1 | 1281186 | 562 | Escherichia coli UMEA 3121-1 | UMEA 3121-1 | Proteobacteria | KE701594.1 | 3 | EQW80283.1 | 18267 |
| 1999 | GCA_000460235.1 | 1281187 | 562 | Escherichia coli UMEA 3122-1 | UMEA 3122-1 | Proteobacteria | KE701612.1 | 3 | EQW87486.1 | 18270 |
| 2000 | GCA_000460415.1 | 1281197 | 562 | Escherichia coli UMEA 3159-1 | UMEA 3159-1 | Proteobacteria | KE701731.1 | 3 | EQX12316.1 | 18273 |
| 2001 | GCA_000460515.1 | 1281202 | 562 | Escherichia coli UMEA 3172-1 | UMEA 3172-1 | Proteobacteria | KE701794.1 | 3 | EQX35785.1 | 18276 |
| 2002 | GCA_000460575.1 | 1281205 | 562 | Escherichia coli UMEA 3175-1 | UMEA 3175-1 | Proteobacteria | KE701853.1 | 3 | EQX43608.1 | 18279 |
| 2003 | GCA_000460655.1 | 1281209 | 562 | Escherichia coli UMEA 3185-1 | UMEA 3185-1 | Proteobacteria | KE701908.1 | 3 | EQX66169.1 | 18282 |
| 2004 | GCA_000460695.1 | 1281211 | 562 | Escherichia coli UMEA 3193-1 | UMEA 3193-1 | Proteobacteria | KE701963.1 | 3 | EQX76187.1 | 18285 |
| 2005 | GCA_000460875.1 | 1281220 | 562 | Escherichia coli UMEA 3216-1 | UMEA 3216-1 | Proteobacteria | KE702085.1 | 3 | EQY22299.1 | 18288 |
| 2006 | GCA_000460915.1 | 1281222 | 562 | Escherichia coli UMEA 3220-1 | UMEA 3220-1 | Proteobacteria | KE702109.1 | 3 | EQY33196.1 | 18291 |
| 2007 | GCA_000460955.1 | 1281224 | 562 | Escherichia coli UMEA 3222-1 | UMEA 3222-1 | Proteobacteria | KE702144.1 | 3 | EQY45032.1 | 18294 |
| 2008 | GCA_000461035.1 | 1281228 | 562 | Escherichia coli UMEA 3244-1 | UMEA 3244-1 | Proteobacteria | KE702194.1 | 3 | EQY55714.1 | 18297 |
| 2009 | GCA_000461055.1 | 1281230 | 562 | Escherichia coli UMEA 3257-1 | UMEA 3257-1 | Proteobacteria | KE702215.1 | 3 | EQY72171.1 | 18300 |
| 2010 | GCA_000461335.1 | 1281248 | 562 | Escherichia coli UMEA 3391-1 | UMEA 3391-1 | Proteobacteria | KE702394.1 | 3 | EQZ19658.1 | 18303 |
| 2011 | GCA_000461375.1 | 1281252 | 562 | Escherichia coli UMEA 3585-1 | UMEA 3585-1 | Proteobacteria | KE702412.1 | 3 | EQZ26675.1 | 18306 |
| 2012 | GCA_000461435.1 | 1281255 | 562 | Escherichia coli UMEA 3617-1 | UMEA 3617-1 | Proteobacteria | KE702447.1 | 3 | EQZ41390.1 | 18309 |
| 2013 | GCA_000461655.1 | 1281268 | 562 | Escherichia coli UMEA 3707-1 | UMEA 3707-1 | Proteobacteria | KE702569.1 | 3 | EQZ91637.1 | 18312 |
| 2014 | GCA_000461815.1 | 1281276 | 562 | Escherichia coli UMEA 3955-1 | UMEA 3955-1 | Proteobacteria | KE702703.1 | 3 | ERA32823.1 | 18315 |
| 2015 | GCA_000461835.1 | 1281277 | 562 | Escherichia coli UMEA 4075-1 | UMEA 4075-1 | Proteobacteria | KE702710.1 | 3 | ERA36348.1 | 18318 |
| 2016 | GCA_000463605.1 | 1281257 | 562 | Escherichia coli UMEA 3652-1 | UMEA 3652-1 | Proteobacteria | KE705271.1 | 3 | ERF54134.1 | 18321 |
| 2017 | GCA_000488075.1 | 1281249 | 562 | Escherichia coli UMEA 3426-1 | UMEA 3426-1 | Proteobacteria | KI530636.1 | 3 | ESK18132.1 | 18324 |
| 2018 | GCA_000488095.1 | 1281234 | 562 | Escherichia coli UMEA 3290-1 | UMEA 3290-1 | Proteobacteria | KI530644.1 | 3 | ESK18554.1 | 18327 |
| 2019 | GCA_000488115.1 | 1281263 | 562 | Escherichia coli UMEA 3693-1 | UMEA 3693-1 | Proteobacteria | KI530660.1 | 3 | ESK29586.1 | 18330 |
| 2020 | GCA_000488315.1 | 1268980 | 562 | Escherichia coli 907391 | 907391 | Proteobacteria | KI530980.1 | 3 | ESC95631.1 | 18333 |
| 2021 | GCA_000488475.1 | 1268990 | 562 | Escherichia coli 907892 | 907892 | Proteobacteria | KI532243.1 | 3 | ESD43645.1 | 18336 |
| 2022 | GCA_000488795.1 | 1269010 | 562 | Escherichia coli 910096-2 | 910096-2 | Proteobacteria | KI534866.1 | 3 | ESE27410.1 | 18339 |
| 2023 | GCA_000488815.1 | 1269008 | 562 | Escherichia coli A25922R | A25922R | Proteobacteria | KI535058.1 | 3 | ESE32327.1 | 18342 |
| 2024 | GCA_000488835.1 | 1269009 | 562 | Escherichia coli A35218R | A35218R | Proteobacteria | KI535180.1 | 3 | ESE38128.1 | 18345 |
| 2025 | GCA_000494975.1 | 1281023 | 562 | Escherichia coli HVH 86 (4-7026218) | HVH 86 (4-7026218) | Proteobacteria | KI538693.1 | 3 | ESP10882.1 | 18348 |
| 2026 | GCA_000494975.1 | 1281023 | 562 | Escherichia coli HVH 86 (4-7026218) | HVH 86 (4-7026218) | Proteobacteria | KI538695.1 | 3 | ESP23171.1 | 18351 |
| 2027 | GCA_000506445.2 | 1389417 | 562 | Escherichia coli LAU-EC6 | LAU-EC6 | Proteobacteria | AYNF01000074.1 | 3 | ETE11169.1 | 18354 |
| 2028 | GCA_000507685.1 | 1281250 | 562 | Escherichia coli UMEA 3489-1 | UMEA 3489-1 | Proteobacteria | KI669372.1 | 3 | ETF35984.1 | 18357 |
| 2029 | GCA_000633655.1 | 1445863 | 562 | Escherichia coli BIDMC 83 | BIDMC 83 | Proteobacteria | KK214043.1 | 3 | EZQ63564.1 | 18360 |
| 2030 | GCA_000692475.1 | 1438673 | 562 | Escherichia coli BIDMC 65 | BIDMC 65 | Proteobacteria | KK736329.1 | 3 | KDG03094.1 | 18363 |
| 2031 | GCA_000696545.1 | 562 | 562 | Escherichia coli | 2009-46 | Proteobacteria | JJNT01000018.1 | 3 | KD090448.1 | 18366 |
| 2032 | GCA_000714595.1 | 316435 | 562 | Escherichia coli Nissle 1917 | Nissle 1917 | Proteobacteria | CP007799.1 | 3 | AID79061.1 | 18369 |
| 2033 | GCA_000743255.1 | 1322345 | 562 | Escherichia coli ATCC 25922 | ATCC 25922 | Proteobacteria | CP009072.1 | 3 | AIL16531.1 | 18372 |
| 2034 | GCA_001030285.1 | 562 | 562 | Escherichia coli | BWH59 | Proteobacteria | KQ087625.1 | 3 | KLX17105.1 | 18375 |
| 2035 | GCA_001030435.1 | 562 | 562 | Escherichia coli | MGH122 | Proteobacteria | KQ087876.1 | 3 | KLX44352.1 | 18378 |
| 2036 | GCA_001030445.1 | 562 | 562 | Escherichia coli | BIDMC97 | Proteobacteria | KQ087859.1 | 3 | KLX45653.1 | 18381 |
| 2037 | GCA_001280385.1 | 562 | 562 | Escherichia coli | SF-166 | Proteobacteria | CP012633.1 | 3 | ALD34922.1 | 18384 |
| 2038 | GCA_001463115.1 | 562 | 562 | Escherichia coli | 50664179 | Proteobacteria | LNHU01000095.1 | 3 | KSY02659.1 | 18387 |
| 2039 | GCA_001518355.1 | 562 | 562 | Escherichia coli | GN02748 | Proteobacteria | LQZV01000057.1 | 3 | KUR38256.1 | 18390 |
| 2040 | GCA_001519115.1 | 562 | 562 | Escherichia coli | GN02007 | Proteobacteria | LQRW01000143.1 | 3 | KUR81832.1 | 18393 |
| 2041 | GCA_001519125.1 | 562 | 562 | Escherichia coli | GN02009 | Proteobacteria | LQRX01000141.1 | 3 | KUR94071.1 | 18396 |
| 2042 | GCA_001519215.1 | 562 | 562 | Escherichia coli | GN02045 | Proteobacteria | LQRY01000066.1 | 3 | KUR99612.1 | 18399 |
| 2043 | GCA_001519315.1 | 562 | 562 | Escherichia coli | GN02183 | Proteobacteria | LQSM01000041.1 | 3 | KUS68705.1 | 18402 |
| 2044 | GCA_001519365.1 | 562 | 562 | Escherichia coli | GN02175 | Proteobacteria | LQSL01000095.1 | 3 | KUS65754.1 | 18405 |
| 2045 | GCA_001519435.1 | 562 | 562 | Escherichia coli | GN02050 | Proteobacteria | LQRZ01000151.1 | 3 | KUS06588.1 | 18408 |
| 2046 | GCA_001519485.1 | 562 | 562 | Escherichia coli | GN03398 | Proteobacteria | LQVU01000143.1 | 3 | KUW86271.1 | 18411 |
| 2047 | GCA_001519595.1 | 562 | 562 | Escherichia coli | GN02323 | Proteobacteria | LQTA01000215.1 | 3 | KUT33561.1 | 18414 |
| 2048 | GCA_001519645.1 | 562 | 562 | Escherichia coli | GN02639 | Proteobacteria | LQUW01000126.1 | 3 | KUV71918.1 | 18417 |
| 2049 | GCA_001519895.1 | 562 | 562 | Escherichia coli | GN02345 | Proteobacteria | LQTD01000025.1 | 3 | KUT65775.1 | 18420 |
| 2050 | GCA_001520055.1 | 562 | 562 | Escherichia coli | GN02392 | Proteobacteria | LQTM01000161.1 | 3 | KUT92654.1 | 18423 |
| 2051 | GCA_001520195.1 | 562 | 562 | Escherichia coli | GN02627 | Proteobacteria | LQUU01000218.1 | 3 | KUV55766.1 | 18426 |
| 2052 | GCA_001520215.1 | 562 | 562 | Escherichia coli | GN02529 | Proteobacteria | LQUG01000209.1 | 3 | KUU92987.1 | 18429 |
| 2053 | GCA_001520555.1 | 562 | 562 | Escherichia coli | GN02590 | Proteobacteria | LQUP01000306.1 | 3 | KUV32574.1 | 18432 |
| 2054 | GCA_001520715.1 | 562 | 562 | Escherichia coli | GN03545 | Proteobacteria | LQVY01000168.1 | 3 | KUX05523.1 | 18435 |
| 2055 | GCA_001520735.1 | 562 | 562 | Escherichia coli | GN03496 | Proteobacteria | LQVX01000201.1 | 3 | KUX02162.1 | 18438 |
| 2056 | GCA_001520815.1 | 562 | 562 | Escherichia coli | GN02766 | Proteobacteria | LQVG01000173.1 | 3 | KUW18188.1 | 18441 |
| 2057 | GCA_001520895.1 | 562 | 562 | Escherichia coli | GN02411 | Proteobacteria | LQTP01000196.1 | 3 | KUU04902.1 | 18444 |
| 2058 | GCA_001521015.1 | 562 | 562 | Escherichia coli | GN02867 | Proteobacteria | LQVL01000165.1 | 3 | KUW44250.1 | 18447 |
| 2059 | GCA_001521155.1 | 562 | 562 | Escherichia coli | GN02787 | Proteobacteria | LQVJ01000229.1 | 3 | KUW29415.1 | 18450 |
| 2060 | GCA_001521225.1 | 562 | 562 | Escherichia coli | GN02350 | Proteobacteria | LQTE01000122.1 | 3 | KUT58679.1 | 18453 |
| 2061 | GCA_001521315.1 | 562 | 562 | Escherichia coli | GN03786 | Proteobacteria | LQWD01000068.1 | 3 | KUX37210.1 | 18456 |
| 2062 | GCA_001521355.1 | 562 | 562 | Escherichia coli | GN02547 | Proteobacteria | LQUL01000230.1 | 3 | KUV14410.1 | 18459 |
| 2063 | GCA_001521455.1 | 562 | 562 | Escherichia coli | GN04262 | Proteobacteria | LQWM01000080.1 | 3 | KUX73639.1 | 18462 |
| 2064 | GCA_001521575.1 | 562 | 562 | Escherichia coli | GN03324 | Proteobacteria | LQVT01000030.1 | 3 | KUW86783.1 | 18465 |
| 2065 | GCA_001521635.1 | 562 | 562 | Escherichia coli | GN04153 | Proteobacteria | LQWI01000317.1 | 3 | KUX51102.1 | 18468 |
| 2066 | GCA_001524885.1 | 562 | 562 | Escherichia coli | GN02609 | Proteobacteria | LRA001000013.1 | 3 | KVI42424.1 | 18471 |
| 2067 | GCA_001524905.1 | 562 | 562 | Escherichia coli | GN02487 | Proteobacteria | LRAN01000026.1 | 3 | KVI22808.1 | 18474 |
| 2068 | GCA_001562835.1 | 562 | 562 | Escherichia coli | JPH264 | Proteobacteria | KQ961179.1 | 3 | KXG92284.1 | 18477 |
| 2069 | GCA_001571575.1 | 562 | 562 | Escherichia coli | UM131 | Proteobacteria | LRX001000021.1 | 3 | KXL04869.1 | 18480 |
| 2070 | GCA_001571585.1 | 562 | 562 | Escherichia coli | UM149 | Proteobacteria | LRXN01000040.1 | 3 | KXL27378.1 | 18483 |
| 2071 | GCA_001571645.1 | 562 | 562 | Escherichia coli | CD44 | Proteobacteria | LRXP01000062.1 | 3 | KXL05683.1 | 18486 |
| 2072 | GCA_001571725.1 | 562 | 562 | Escherichia coli | UM128 | Proteobacteria | LRXU01000008.1 | 3 | KXL13115.1 | 18489 |
| 2073 | GCA_001471755.1 | 564 | 564 | Escherichia fergusonii | FDAARGOS_170 | Proteobacteria | LORS01000001.1 | 3 | KTK75705.1 | 18492 |
| 2074 | GCA_000415405.1 | 1244143 | 1351 | Enterococcus faecalis VC1B-1 | VC1B-1 | Firmicutes | KE352639.1 | 3 | EPI32466.1 | 18495 |
| 2075 | GCA_000519685.1 | 1391483 | 1351 | Enterococcus faecalis EnGen0413 | EnGen0413 | Firmicutes | KI913017.1 | 3 | ETU24520.1 | 18498 |
| 2076 | GCA_000322325.1 | 1138929 | 1352 | Enterococcus faecium EnGen0048 | E4215 | Firmicutes | KB030000.1 | 3 | ELB67022.1 | 18501 |
| 2077 | GCA_000371505.1 | 999408 | 1531 | [Clostridium] clostridioforme 90A8 | 90A8 | Firmicutes | KB850978.1 | 3 | ENZ13057.1 | 18504 |
| 2078 | GCA_000487575.2 | 984211 | 28901 | Salmonella enterica subsp. | ATCC BAA-1592 | Proteobacteria | CP007531.1 | 3 | AHW13890.1 | 18507 |
| | | | | enterica serovar Anatum str. | | | | | | |
| | | | | ATCC BAA-1592 | | | | | | |
| 2079 | GCA_000495295.1 | 1321366 | 28901 | Salmonella enterica subsp. | S-70 | Proteobacteria | AQQN01000068.1 | 3 | ESP72121.1 | 18510 |
| | | | | enterica serovar Saintpaul | | | | | | |
| | | | | str. S-70 | | | | | | |
| 2080 | GCA_000495315.1 | 1323580 | 28901 | Salmonella enterica subsp. | S-76 | Proteobacteria | AROZ01000126.1 | 3 | ESP76604.1 | 18513 |
| | | | | enterica serovar Oranienburg | | | | | | |
| | | | | str. S-76 | | | | | | |
| 2081 | GCA_000748865.1 | 1173435 | 28901 | Salmonella enterica subsp. | CFSAN000207 | Proteobacteria | JRDQ01000027.1 | 3 | KFT73591.1 | 18516 |
| | | | | enterica serovar Bareilly | | | | | | |
| | | | | str. CFSAN000207 | | | | | | |
| 2082 | GCA_000806625.1 | 59201 | 28901 | Salmonella enterica subsp. | CFSAN024764 | Proteobacteria | JWQO01000015.1 | 3 | KHP33099.1 | 18519 |
| | | | | enterica | | | | | | |
| 2083 | GCA_000806925.1 | 59201 | 28901 | Salmonella enterica subsp. | CFSAN024763 | Proteobacteria | JWQP01000009.1 | 3 | KHP34815.1 | 18522 |
| | | | | enterica | | | | | | |
| 2084 | GCA_000988525.1 | 1454585 | 28901 | Salmonella enterica subsp. | USDA-ARS- | Proteobacteria | CP007584.1 | 3 | AKF88465.1 | 18525 |
| | | | | enterica serovar Anatum | USMARC-1735 | | | | | |
| | | | | str. USDA-ARS-USMARC-1735 | | | | | | |
| 2085 | GCA_001241205.1 | 58712 | 28901 | Salmonella enterica subsp. | CVM N44698 | Proteobacteria | LHFG01000018.1 | 3 | KNK74744.1 | 18528 |
| | | | | enterica serovar Anatum | | | | | | |
| 2086 | GCA_001247355.1 | 58712 | 28901 | Salmonella enterica subsp. | CVM N51307 | Proteobacteria | LHON01000003.1 | 3 | KNW86449.1 | 18531 |
| | | | | enterica serovar Anatum | | | | | | |
| 2087 | GCA_001447095.1 | 58712 | 28901 | Salmonella enterica subsp. | GT-38 | Proteobacteria | CP013226.1 | 3 | ALO88020.1 | 18534 |
| | | | | enterica serovar Anatum | | | | | | |
| 2088 | GCA_001475375.1 | 28901 | 28901 | Salmonella enterica | CVM 43842 | Proteobacteria | JYTH01000003.1 | 3 | KTQ21993.1 | 18537 |
| 2089 | GCA_001478355.1 | 58712 | 28901 | Salmonella enterica subsp. | CVM N38876 | Proteobacteria | JYXM01000005.1 | 3 | KUB19293.1 | 18540 |
| | | | | enterica serovar Anatum | | | | | | |
| 2090 | GCA_001479145.1 | 58712 | 28901 | Salmonella enterica subsp. | CVM N31410 | Proteobacteria | JYVK01000019.1 | 3 | KTY61367.1 | 18543 |
| | | | | enterica serovar Anatum | | | | | | |
| 2091 | GCA_001479905.1 | 192955 | 28901 | Salmonella enterica subsp. | CVM N38870 | Proteobacteria | JYXJ01000004.1 | 3 | KUB06249.1 | 18546 |
| | | | | enterica serovar Kentucky | | | | | | |
| 2092 | GCA_001481105.1 | 58712 | 28901 | Salmonella enterica subsp. | CVM N42473 | Proteobacteria | JYZV01000014.1 | 3 | KUD94944.1 | 18549 |
| | | | | enterica serovar Anatum | | | | | | |
| 2093 | GCA_001569785.1 | 28901 | 28901 | Salmonella enterica | NGUA13 | Proteobacteria | BCOL01000077.1 | 3 | GAR53165.1 | 18552 |
| 2094 | GCA_000154805.1 | 428126 | 29348 | [Clostridium] spiroforme | DSM 1552 | Firmicutes | DS562851.1 | 3 | EDS74761.1 | 18555 |
| | | | | DSM 1552 | | | | | | |
| 2095 | GCA_001483145.1 | 172045 | 172045 | Elizabethkingia miricola | EM_CHUV | Bacteroidetes | LIQC01000001.1 | 3 | KUG13796.1 | 18558 |
| 2096 | GCA_000273015.1 | 997874 | 246787 | Bacteroides cellulosilyticus | CL02T12C19 | Bacteroidetes | JH724089.1 | 3 | EIY23905.1 | 18561 |
| | | | | CL02T12C19 | | | | | | |
| 2097 | GCA_000403255.2 | 397288 | 397288 | Lachnospiraceae bacterium 3-1 | 3-1 | Firmicutes | KE159581.1 | 3 | EOS20511.1 | 18564 |
| 2098 | GCA_000157115.2 | 469598 | 469598 | Escherichia sp. 3_2_53FAA | 3_2_53FAA | Proteobacteria | KQ235746.1 | 3 | EEH86957.1 | 18567 |
| 2099 | GCA_000437535.1 | 1263103 | 1263103 | Prevotella stercorea CAG:629 | | Bacteroidetes | FR899171.1 | 3 | CDE34946.1 | 18570 |
| 2100 | GCA_000486445.2 | 938141 | 28901 | Salmonella enterica subsp. | ATCC 15791 | Proteobacteria | CP012349.1 | 4 | AKW15253.1 | 18573 |
| | | | | enterica serovar Sloterdijk | | | | | | |
| | | | | str. ATCC 15791 | | | | | | |

(Part B)

| | | L | | | | P | | | | T | | | | X |
|---|---|---|---|---|---|---|---|---|---|---|---|---|---|---|
| | | ThsA | | | | ThsB (i) | | | | ThsB (ii) | | | | ThsB (iii) |
| | K | protein | M | N | O | protein | Q | R | S | protein | U | V | W | protein |
| | DNA SEQ | length | ThsB (i) | PRT SEQ | DNA SEQ | length | ThsB (ii) | PRT SEQ | DNA SEQ | length | ThsB (iii) | PRT SEQ | DNA SEQ | length |
| 1 | ID NO: | (aa) | protein_accession | ID NO: | ID NO: | (aa) | protein_accession | ID NO: | ID NO: | (aa) | protein_accession | ID NO: | ID NO: | (aa) |
| 2 | 18577 | 478 | EAN10000.1 | 13221 | 18578 | 161 | EAN09998.1 | 13222 | 18579 | 283 | | | | |
| 3 | 18580 | 477 | EAZ85482.1 | 13224 | 18581 | 226 | | | | | | | | |
| 4 | 18582 | 478 | EFF33949.1 | 13226 | 18583 | 161 | EFF33951.1 | 13227 | 18584 | 283 | | | | |
| 5 | 18585 | 478 | EFF30625.1 | 13229 | 18586 | 161 | EFF30623.1 | 13230 | 18587 | 283 | | | | |
| 6 | 18588 | 461 | EFA90643.1 | 13232 | 18589 | 165 | | | | | | | | |
| 7 | 18590 | 474 | EFM65151.1 | 13234 | 18591 | 165 | | | | | | | | |
| 8 | 18592 | 471 | EFF51767.1 | 13236 | 18593 | 182 | EFF51769.1 | 13237 | 18594 | 203 | | | | |
| 9 | 18595 | 500 | AAP11367.1 | 13239 | 18596 | 206 | | | | | | | | |
| 10 | 18597 | 473 | EFM08815.1 | 13241 | 18598 | 166 | EFM08816.1 | 13242 | 18599 | 203 | | | | |
| 11 | 18600 | 466 | KND82315.1 | 13244 | 18601 | 160 | KND82316.1 | 13245 | 18602 | 214 | | | | |
| 12 | 18603 | 466 | KOE55211.1 | 13247 | 18604 | 160 | KOE55212.1 | 13248 | 18605 | 214 | | | | |
| 13 | 18606 | 478 | EHM35988.1 | 13250 | 18607 | 161 | EHM35990.1 | 13251 | 18608 | 283 | | | | |
| 14 | 18609 | 473 | EPE98430.1 | 13253 | 18610 | 162 | | | | | | | | |
| 15 | 18611 | 217 | EGV07223.1 | 13255 | 18612 | 208 | EGV07230.1 | 13256 | 18613 | 164 | | | | |
| 16 | 18614 | 469 | EHD29826.1 | 13258 | 18615 | 206 | EHD29828.1 | 13259 | 18616 | 310 | | | | |
| 17 | 18617 | 375 | EHM75406.1 | 13261 | 18618 | 202 | EHM75402.1 | 13262 | 18619 | 308 | | | | |
| 18 | 18620 | 466 | EHK91560.1 | 13264 | 18621 | 69 | EHK91561.1 | 13265 | 18622 | 214 | | | | |
| 19 | 18623 | 489 | EOR48147.1 | 13267 | 18624 | 202 | EOR48148.1 | 13268 | 18625 | 344 | | | | |
| 20 | 18626 | 470 | EID19241.1 | 13270 | 18627 | 206 | EID19242.1 | 13271 | 18628 | 388 | | | | |
| 21 | 18629 | 381 | EIA14314.1 | 13273 | 18630 | 202 | EIA14315.1 | 13274 | 18631 | 344 | | | | |
| 22 | 18632 | 159 | EIC95415.1 | 13276 | 18633 | 122 | | | | | | | | |
| 23 | 18634 | 476 | EKS70340.1 | 13278 | 18635 | 162 | | | | | | | | |
| 24 | 18636 | 466 | KOE52659.1 | 13280 | 18637 | 214 | KOE52660.1 | 13281 | 18638 | 160 | | | | |
| 25 | 18639 | 466 | KOE56125.1 | 13283 | 18640 | 214 | KOE56126.1 | 13284 | 18641 | 160 | | | | |
| 26 | 18642 | 465 | EIJ69684.1 | 13286 | 18643 | 164 | EIJ69602.1 | 13287 | 18644 | 208 | | | | |
| 27 | 18645 | 208 | EIL59627.1 | 13289 | 18646 | 155 | | | | | | | | |
| 28 | 18647 | 477 | EJH65284.1 | 13291 | 18648 | 97 | | | | | | | | |
| 29 | 18649 | 473 | EPV35488.1 | 13293 | 18650 | 165 | | | | | | | | |
| 30 | 18651 | 499 | CAN93963.1 | 13295 | 18652 | 956 | | | | | | | | |
| 31 | 18653 | 483 | CAQ50580.1 | 13297 | 18654 | 202 | CAQ50581.1 | 13298 | 18655 | 308 | | | | |
| 32 | 18656 | 482 | EKF39861.1 | 13300 | 18657 | 162 | EKF39859.1 | 13301 | 18658 | 184 | | | | |
| 33 | 18659 | 477 | ETE93553.1 | 13303 | 18660 | 226 | | | | | | | | |
| 34 | 18661 | 477 | ETE97367.1 | 13305 | 18662 | 226 | | | | | | | | |
| 35 | 18663 | 479 | EPC81227.1 | 13307 | 18664 | 210 | | | | | | | | |
| 36 | 18665 | 478 | ELI26318.1 | 13309 | 18666 | 203 | | | | | | | | |
| 37 | 18667 | 478 | ELJ29819.1 | 13311 | 18668 | 203 | | | | | | | | |
| 38 | 18669 | 472 | EME14865.1 | 13313 | 18670 | 160 | EME14867.1 | 13314 | 18671 | 194 | | | | |
| 39 | 18672 | 484 | EMF02810.1 | 13316 | 18673 | 163 | | | | | | | | |
| 40 | 18674 | 484 | BAO35115.1 | 13318 | 18675 | 163 | | | | | | | | |
| 41 | 18676 | 560 | BAO91364.1 | 13320 | 18677 | 485 | | | | | | | | |
| 42 | 18678 | 482 | BAQ74701.1 | 13322 | 18679 | 158 | BAQ74699.1 | 13323 | 18680 | 134 | BAQ74700.1 | 13324 | 18681 | 177 |
| 43 | 18683 | 480 | ENZ83251.1 | 13327 | 18684 | 163 | ENZ83249.1 | 13328 | 18685 | 194 | | | | |
| 44 | 18686 | 480 | EQB07553.1 | 13330 | 18687 | 155 | | | | | | | | |
| 45 | 18688 | 476 | EPJ82188.1 | 13332 | 18689 | 160 | | | | | | | | |
| 46 | 18690 | 480 | EPR14529.1 | 13334 | 18691 | 155 | | | | | | | | |
| 47 | 18692 | 483 | EQM91606.1 | 13336 | 18693 | 202 | EQM91605.1 | 13337 | 18694 | 344 | | | | |
| 48 | 18695 | 483 | EPZ04958.1 | 13339 | 18696 | 202 | EPZ04959.1 | 13340 | 18697 | 344 | | | | |
| 49 | 18698 | 483 | EPZ06673.1 | 13342 | 18699 | 202 | EPZ06672.1 | 13343 | 18700 | 344 | | | | |
| 50 | 18701 | 375 | EPZ03689.1 | 13345 | 18702 | 202 | EPZ03688.1 | 13346 | 18703 | 344 | | | | |
| 51 | 18704 | 375 | EPZ11498.1 | 13348 | 18705 | 202 | EPZ11497.1 | 13349 | 18706 | 308 | | | | |
| 52 | 18707 | 231 | EQC60102.1 | 13351 | 18708 | 165 | | | | | | | | |
| 53 | 18709 | 473 | EQG20076.1 | 13353 | 18710 | 141 | | | | | | | | |
| 54 | 18711 | 473 | EQK23002.1 | 13355 | 18712 | 165 | | | | | | | | |
| 55 | 18713 | 482 | ETR95050.1 | 13357 | 18714 | 164 | ETR94961.1 | 13358 | 18715 | 385 | ETR95013.1 | 13359 | 18716 | 111 |
| 56 | 18719 | 481 | EQL89246.1 | 13363 | 18720 | 104 | EQL89247.1 | 13364 | 18721 | 191 | | | | |
| 57 | 18722 | 484 | ERH73217.1 | 13366 | 18723 | 163 | | | | | | | | |
| 58 | 18724 | 473 | ERT62591.1 | 13368 | 18725 | 165 | | | | | | | | |
| 59 | 18726 | 487 | ERT61533.1 | 13370 | 18727 | 163 | ERT61545.1 | 13371 | 18728 | 376 | | | | |
| 60 | 18729 | 473 | ERT58461.1 | 13373 | 18730 | 165 | | | | | | | | |
| 61 | 18731 | 482 | ERT19167.1 | 13375 | 18732 | 158 | ERT19165.1 | 13376 | 18733 | 179 | ERT19166.1 | 13377 | 18734 | 183 |
| 62 | 18736 | 487 | ESR69981.1 | 13380 | 18737 | 167 | ESR69983.1 | 13381 | 18738 | 183 | | | | |
| 63 | 18739 | 480 | KAJ84752.1 | 13383 | 18740 | 141 | KAJ84753.1 | 13384 | 18741 | 206 | | | | |
| 64 | 18742 | 479 | KAJ82224.1 | 13386 | 18743 | 206 | | | | | | | | |
| 65 | 18744 | 474 | ETD82033.1 | 13388 | 18745 | 165 | | | | | | | | |
| 66 | 18746 | 489 | ESZ88709.1 | 13390 | 18747 | 168 | ESZ88707.1 | 13391 | 18748 | 184 | | | | |
| 67 | 18749 | 478 | ETJ99317.1 | 13393 | 18750 | 203 | | | | | | | | |
| 68 | 18751 | 221 | KYK73941.1 | 13395 | 18752 | 160 | KYK73942.1 | 13396 | 18753 | 214 | | | | |
| 69 | 18754 | 468 | ETO98875.1 | 13398 | 18755 | 166 | | | | | | | | |
| 70 | 18756 | 456 | GAE27471.1 | 13400 | 18757 | 161 | | | | | | | | |
| 71 | 18758 | 451 | GAM02794.1 | 13402 | 18759 | 143 | GAM02793.1 | 13403 | 18760 | 186 | | | | |
| 72 | 18761 | 477 | CAE30981.1 | 13405 | 18762 | 193 | CAE30983.1 | 13406 | 18763 | 165 | | | | |
| 73 | 18764 | 477 | CCP18524.1 | 13408 | 18765 | 146 | CCP18522.1 | 13409 | 18766 | 307 | | | | |
| 74 | 18767 | 480 | CDZ53066.1 | 13411 | 18768 | 159 | | | | | | | | |
| 75 | 18769 | 468 | CEF52296.1 | 13413 | 18770 | 153 | | | | | | | | |
| 76 | 18771 | 237 | CJD59502.1 | 13415 | 18772 | 248 | | | | | | | | |
| 77 | 18773 | 500 | EEL09526.1 | 13417 | 18774 | 206 | | | | | | | | |
| 78 | 18775 | 476 | EEL84781.1 | 13419 | 18776 | 192 | EEL84783.1 | 13420 | 18777 | 139 | | | | |
| 79 | 18778 | 476 | EEL90115.1 | 13422 | 18779 | 192 | EEL90113.1 | 13423 | 18780 | 139 | | | | |
| 80 | 18781 | 473 | EFH92718.1 | 13425 | 18782 | 165 | | | | | | | | |
| 81 | 18783 | 486 | EFR92172.1 | 13427 | 18784 | 202 | | | | | | | | |
| 82 | 18785 | 487 | EIK62341.1 | 13429 | 18786 | 167 | EIK59131.1 | 13430 | 18787 | 192 | | | | |
| 83 | 18788 | 481 | ABB40970.1 | 13432 | 18789 | 146 | ABB40968.1 | 13433 | 18790 | 180 | ABB40962.1 | 13434 | 18791 | 262 |
| 84 | 18792 | 480 | ABD68806.1 | 13436 | 18793 | 157 | | | | | | | | |
| 85 | 18794 | 479 | ABR91779.1 | 13438 | 18795 | 163 | ABR91881.1 | 13439 | 18796 | 197 | | | | |
| 86 | 18797 | 477 | ABI42032.1 | 13441 | 18798 | 164 | ABI42037.1 | 13442 | 18799 | 160 | | | | |
| 87 | 18800 | 466 | ABN74272.1 | 13444 | 18801 | 165 | ABN74273.1 | 13445 | 18802 | 214 | | | | |
| 88 | 18803 | 549 | ABO35463.1 | 13447 | 18804 | 171 | | | | | | | | |
| 89 | 18805 | 484 | ABP73210.1 | 13449 | 18806 | 165 | ABP73208.1 | 13450 | 18807 | 183 | | | | |
| 90 | 18808 | 478 | ABR34953.1 | 13452 | 18809 | 229 | ABR34957.1 | 13453 | 18810 | 202 | ABR34959.1 | 13454 | 18811 | 166 |
| 91 | 18813 | 1197 | ABR65054.1 | 13457 | 18814 | 641 | | | | | | | | |
| 92 | 18815 | 482 | ACA73844.1 | 13459 | 18816 | 158 | ACA73842.1 | 13460 | 18817 | 179 | ACA73843.1 | 13461 | 18818 | 183 |
| 93 | 18820 | 476 | ACB66893.1 | 13464 | 18821 | 135 | ACB66892.1 | 13465 | 18822 | 197 | | | | |
| 94 | 18823 | 342 | ACX81592.1 | 13467 | 18824 | 214 | ACX81593.1 | 13468 | 18825 | 160 | | | | |
| 95 | 18826 | 479 | ACZ11006.1 | 13470 | 18827 | 202 | | | | | | | | |
| 96 | 18828 | 325 | ADB76072.1 | 13472 | 18829 | 421 | ADB76078.1 | 13473 | 18830 | 793 | | | | |
| 97 | 18831 | 481 | ADY67728.1 | 13475 | 18832 | 162 | ADY67726.1 | 13476 | 18833 | 130 | ADY67729.1 | 13477 | 18834 | 465 |
| 98 | 18835 | 293 | ADP84994.1 | 13479 | 18836 | 535 | | | | | | | | |
| 99 | 18837 | 345 | AEB29796.1 | 13481 | 18838 | 182 | AEB29798.1 | 13482 | 18839 | 194 | | | | |
| 100 | 18840 | 482 | AEA61150.1 | 13484 | 18841 | 161 | AEA61151.1 | 13485 | 18842 | 193 | AEA61157.1 | 13486 | 18843 | 155 |
| 101 | 18846 | 381 | AFH70384.1 | 13490 | 18847 | 202 | AFH70385.1 | 13491 | 18848 | 344 | | | | |
| 102 | 18849 | 466 | AEW76522.1 | 13493 | 18850 | 214 | AEW76523.1 | 13494 | 18851 | 160 | | | | |
| 103 | 18852 | 500 | AEW57569.1 | 13496 | 18853 | 206 | | | | | | | | |
| 104 | 18854 | 504 | AFK54068.1 | 13498 | 18855 | 151 | AFK54069.1 | 13499 | 18856 | 206 | AFK54066.1 | 13500 | 18857 | 285 |
| 105 | 18858 | 484 | AIM23121.1 | 13502 | 18859 | 163 | | | | | | | | |
| 106 | 18860 | 476 | AGG89636.1 | 13504 | 18861 | 158 | | | | | | | | |
| 107 | 18862 | 478 | AFK58217.1 | 13506 | 18863 | 161 | AFK58215.1 | 13507 | 18864 | 283 | | | | |
| 108 | 18865 | 443 | AFO49033.1 | 13509 | 18866 | 133 | AFO49034.1 | 13510 | 18867 | 177 | AFO49032.1 | 13511 | 18868 | 282 |
| 109 | 18869 | 483 | AFR74118.1 | 13513 | 18870 | 202 | AFR74119.1 | 13514 | 18871 | 344 | | | | |
| 110 | 18872 | 551 | AHK33007.1 | 13516 | 18873 | 377 | | | | | | | | |
| 111 | 18874 | 484 | AGE19434.1 | 13518 | 18875 | 163 | | | | | | | | |
| 112 | 18876 | 307 | AHZ68663.1 | 13520 | 18877 | 146 | AHZ68658.1 | 13521 | 18878 | 165 | AHZ68664.1 | 13522 | 18879 | 275 |
| 113 | 18880 | 103 | AGM44766.1 | 13524 | 18881 | 155 | | | | | | | | |
| 114 | 18882 | 479 | AGS26694.1 | 13526 | 18883 | 209 | | | | | | | | |
| 115 | 18884 | 484 | AGO56415.1 | 13528 | 18885 | 163 | | | | | | | | |
| 116 | 18886 | 464 | AGP48696.1 | 13530 | 18887 | 158 | AGP48695.1 | 13531 | 18888 | 259 | | | | |
| 117 | 18889 | 478 | AIU00716.1 | 13533 | 18890 | 229 | AIU02169.1 | 13534 | 18891 | 202 | AIU02168.1 | 13535 | 18892 | 166 |
| 118 | 18894 | 480 | AHB77237.1 | 13538 | 18895 | 163 | AHB77238.1 | 13539 | 18896 | 253 | | | | |
| 119 | 18897 | 240 | AJK51072.1 | 13541 | 18898 | 156 | | | | | | | | |
| 120 | 18899 | 474 | AHC80714.1 | 13543 | 18900 | 163 | AHC80713.1 | 13544 | 18901 | 136 | | | | |
| 121 | 18902 | 474 | AHC86146.1 | 13546 | 18903 | 163 | AHC86145.1 | 13547 | 18904 | 136 | | | | |
| 122 | 18905 | 420 | AHN71070.1 | 13549 | 18906 | 214 | AHN71071.1 | 13550 | 18907 | 160 | | | | |
| 123 | 18908 | 466 | AKU28343.1 | 13552 | 18909 | 214 | AKU28344.1 | 13553 | 18910 | 160 | | | | |
| 124 | 18911 | 478 | AJI88525.1 | 13555 | 18912 | 160 | AJI88219.1 | 13556 | 18913 | 360 | | | | |
| 125 | 18914 | 472 | AIQ75143.1 | 13558 | 18915 | 214 | AIQ75145.1 | 13559 | 18916 | 234 | | | | |
| 126 | 18917 | 480 | AIY12609.1 | 13561 | 18918 | 203 | | | | | | | | |
| 127 | 18919 | 478 | AJJ37269.1 | 13563 | 18920 | 160 | AJJ35782.1 | 13564 | 18921 | 360 | | | | |
| 128 | 18922 | 443 | AJG12528.1 | 13566 | 18923 | 158 | AJG12529.1 | 13567 | 18924 | 183 | | | | |
| 129 | 18925 | 480 | AJY68817.1 | 13569 | 18926 | 163 | AJY68816.1 | 13570 | 18927 | 196 | | | | |
| 130 | 18928 | 591 | AJP52228.1 | 13572 | 18929 | 322 | | | | | | | | |
| 131 | 18930 | 481 | AJP42563.1 | 13574 | 18931 | 166 | | | | | | | | |
| 132 | 18932 | 487 | AKK01415.1 | 13576 | 18933 | 167 | AKK01413.1 | 13577 | 18934 | 183 | | | | |
| 133 | 18935 | 487 | AKC95052.1 | 13579 | 18936 | 163 | AKC96108.1 | 13580 | 18937 | 175 | AKC95053.1 | 13581 | 18938 | 376 |
| 134 | 18939 | 288 | AKO52331.1 | 13583 | 18940 | 478 | | | | | | | | |
| 135 | 18941 | 472 | AKV95757.1 | 13585 | 18942 | 195 | | | | | | | | |
| 136 | 18943 | 484 | ALE97362.1 | 13587 | 18944 | 163 | | | | | | | | |
| 137 | 18945 | 485 | ALJ08796.1 | 13589 | 18946 | 148 | ALJ08793.1 | 13590 | 18947 | 175 | ALJ08794.1 | 13591 | 18948 | 184 |
| 138 | 18949 | 466 | AMQ92900.1 | 13593 | 18950 | 161 | AMQ92901.1 | 13594 | 18951 | 214 | | | | |
| 139 | 18952 | 482 | ALL13163.1 | 13596 | 18953 | 148 | ALL13165.1 | 13597 | 18954 | 183 | | | | |
| 140 | 18955 | 103 | ALQ62809.1 | 13599 | 18956 | 155 | ALQ62808.1 | 13600 | 18957 | 192 | | | | |
| 141 | 18958 | 499 | ALS70448.1 | 13602 | 18959 | 202 | ALS70449.1 | 13603 | 18960 | 344 | | | | |
| 142 | 18961 | 489 | ALS73059.1 | 13605 | 18962 | 202 | ALS73060.1 | 13606 | 18963 | 344 | | | | |
| 143 | 18964 | 489 | ALS86456.1 | 13608 | 18965 | 202 | ALS86457.1 | 13609 | 18966 | 344 | | | | |
| 144 | 18967 | 482 | ALZ77340.1 | 13611 | 18968 | 155 | | | | | | | | |
| 145 | 18969 | 103 | ALZ79509.1 | 13613 | 18970 | 155 | ALZ79508.1 | 13614 | 18971 | 192 | | | | |
| 146 | 18972 | 478 | ALZ52746.1 | 13616 | 18973 | 161 | ALZ52748.1 | 13617 | 18974 | 283 | | | | |
| 147 | 18975 | 475 | AMJ40924.1 | 13619 | 18976 | 165 | AMJ40925.1 | 13620 | 18977 | 164 | AMJ40928.1 | 13621 | 18978 | 190 |
| 148 | 18979 | 470 | AMH88750.1 | 13623 | 18980 | 206 | AMH88748.1 | 13624 | 18981 | 388 | | | | |
| 149 | 18982 | 477 | AMK33279.1 | 13626 | 18983 | 101 | | | | | | | | |
| 150 | 18984 | 1269 | CNF27378.1 | 13628 | 18985 | 197 | | | | | | | | |
| 151 | 18986 | 477 | CNF62559.1 | 13630 | 18987 | 146 | CNF62502.1 | 13631 | 18988 | 161 | | | | |
| 152 | 18989 | 477 | CNI81985.1 | 13633 | 18990 | 146 | CNI82037.1 | 13634 | 18991 | 161 | | | | |
| 153 | 18992 | 477 | CNI72014.1 | 13636 | 18993 | 146 | CNI72072.1 | 13637 | 18994 | 161 | | | | |
| 154 | 18995 | 475 | CQR45869.1 | 13639 | 18996 | 160 | | | | | | | | |
| 155 | 18997 | 475 | CQJ38327.1 | 13641 | 18998 | 163 | CQJ38351.1 | 13642 | 18999 | 200 | CQJ38363.1 | 13643 | 19000 | 205 |
| 156 | 19002 | 488 | CRD50733.1 | 13646 | 19003 | 160 | CRD50737.1 | 13647 | 19004 | 224 | CRD50724.1 | 13648 | 19005 | 168 |
| 157 | 19006 | 472 | CRL40089.1 | 13650 | 19007 | 260 | | | | | | | | |
| 158 | 19008 | 473 | CRR05841.1 | 13652 | 19009 | 165 | | | | | | | | |
| 159 | 19010 | 473 | CRR37100.1 | 13654 | 19011 | 165 | | | | | | | | |
| 160 | 19012 | 473 | CRR43597.1 | 13656 | 19013 | 165 | | | | | | | | |
| 161 | 19014 | 473 | CRR34906.1 | 13658 | 19015 | 165 | | | | | | | | |
| 162 | 19016 | 473 | CRR50125.1 | 13660 | 19017 | 165 | | | | | | | | |
| 163 | 19018 | 473 | CRR37378.1 | 13662 | 19019 | 165 | | | | | | | | |
| 164 | 19020 | 473 | CRR01720.1 | 13664 | 19021 | 165 | | | | | | | | |
| 165 | 19022 | 473 | CRR52924.1 | 13666 | 19023 | 165 | | | | | | | | |
| 166 | 19024 | 473 | CRR41677.1 | 13668 | 19025 | 165 | | | | | | | | |
| 167 | 19026 | 473 | CRS57172.1 | 13670 | 19027 | 165 | | | | | | | | |
| 168 | 19028 | 473 | CRS58230.1 | 13672 | 19029 | 165 | | | | | | | | |
| 169 | 19030 | 473 | CRS57785.1 | 13674 | 19031 | 165 | | | | | | | | |
| 170 | 19032 | 473 | CRS58148.1 | 13676 | 19033 | 165 | | | | | | | | |
| 171 | 19034 | 473 | CRS55115.1 | 13678 | 19035 | 165 | | | | | | | | |
| 172 | 19036 | 473 | CRS48573.1 | 13680 | 19037 | 165 | | | | | | | | |
| 173 | 19038 | 473 | CRS51329.1 | 13682 | 19039 | 165 | | | | | | | | |
| 174 | 19040 | 473 | CRS48383.1 | 13684 | 19041 | 165 | | | | | | | | |
| 175 | 19042 | 473 | CRS50250.1 | 13686 | 19043 | 165 | | | | | | | | |
| 176 | 19044 | 473 | CRS50706.1 | 13688 | 19045 | 165 | | | | | | | | |
| 177 | 19046 | 473 | CRS54432.1 | 13690 | 19047 | 165 | | | | | | | | |
| 178 | 19048 | 473 | CRS57073.1 | 13692 | 19049 | 165 | | | | | | | | |
| 179 | 19050 | 473 | CRS58971.1 | 13694 | 19051 | 165 | | | | | | | | |
| 180 | 19052 | 473 | CRS51119.1 | 13696 | 19053 | 165 | | | | | | | | |
| 181 | 19054 | 473 | CRS51969.1 | 13698 | 19055 | 165 | | | | | | | | |
| 182 | 19056 | 473 | CRS77748.1 | 13700 | 19057 | 165 | | | | | | | | |
| 183 | 19058 | 473 | CRS83922.1 | 13702 | 19059 | 165 | | | | | | | | |
| 184 | 19060 | 473 | CRS76799.1 | 13704 | 19061 | 165 | | | | | | | | |
| 185 | 19062 | 473 | CRS78508.1 | 13706 | 19063 | 165 | | | | | | | | |
| 186 | 19064 | 473 | CRS90915.1 | 13708 | 19065 | 165 | | | | | | | | |
| 187 | 19066 | 473 | CRS79984.1 | 13710 | 19067 | 165 | | | | | | | | |
| 188 | 19068 | 473 | CRS84447.1 | 13712 | 19069 | 165 | | | | | | | | |
| 189 | 19070 | 473 | CRS81782.1 | 13714 | 19071 | 165 | | | | | | | | |
| 190 | 19072 | 473 | CRS82426.1 | 13716 | 19073 | 165 | | | | | | | | |
| 191 | 19074 | 473 | CRS93242.1 | 13718 | 19075 | 165 | | | | | | | | |
| 192 | 19076 | 473 | CRT34724.1 | 13720 | 19077 | 165 | | | | | | | | |
| 193 | 19078 | 473 | CRT26954.1 | 13722 | 19079 | 165 | | | | | | | | |
| 194 | 19080 | 473 | CRT43911.1 | 13724 | 19081 | 165 | | | | | | | | |
| 195 | 19082 | 473 | CRT34257.1 | 13726 | 19083 | 165 | | | | | | | | |
| 196 | 19084 | 473 | CRT48110.1 | 13728 | 19085 | 165 | | | | | | | | |
| 197 | 19086 | 473 | CRT41995.1 | 13730 | 19087 | 165 | | | | | | | | |
| 198 | 19088 | 473 | CRT45579.1 | 13732 | 19089 | 165 | | | | | | | | |
| 199 | 19090 | 473 | CRT37671.1 | 13734 | 19091 | 165 | | | | | | | | |
| 200 | 19092 | 473 | CRT44902.1 | 13736 | 19093 | 165 | | | | | | | | |
| 201 | 19094 | 473 | CRT51336.1 | 13738 | 19095 | 165 | | | | | | | | |
| 202 | 19096 | 473 | CRT46016.1 | 13740 | 19097 | 165 | | | | | | | | |
| 203 | 19098 | 473 | CRT46041.1 | 13742 | 19099 | 165 | | | | | | | | |
| 204 | 19100 | 473 | CRT64543.1 | 13744 | 19101 | 165 | | | | | | | | |
| 205 | 19102 | 473 | CRT20963.1 | 13746 | 19103 | 165 | | | | | | | | |
| 206 | 19104 | 473 | CRT55725.1 | 13748 | 19105 | 165 | | | | | | | | |
| 207 | 19106 | 473 | CRT61707.1 | 13750 | 19107 | 165 | | | | | | | | |
| 208 | 19108 | 473 | CRT39213.1 | 13752 | 19109 | 165 | | | | | | | | |
| 209 | 19110 | 473 | CRT66724.1 | 13754 | 19111 | 165 | | | | | | | | |
| 210 | 19112 | 473 | CRT72269.1 | 13756 | 19113 | 165 | | | | | | | | |
| 211 | 19114 | 473 | CRT58958.1 | 13758 | 19115 | 165 | | | | | | | | |
| 212 | 19116 | 473 | CRT49588.1 | 13760 | 19117 | 165 | | | | | | | | |
| 213 | 19118 | 473 | CRT72944.1 | 13762 | 19119 | 165 | | | | | | | | |
| 214 | 19120 | 473 | CRT74928.1 | 13764 | 19121 | 165 | | | | | | | | |
| 215 | 19122 | 473 | CRT61455.1 | 13766 | 19123 | 165 | | | | | | | | |
| 216 | 19124 | 473 | CRT76662.1 | 13768 | 19125 | 165 | | | | | | | | |
| 217 | 19126 | 473 | CRT71352.1 | 13770 | 19127 | 165 | | | | | | | | |
| 218 | 19128 | 473 | CRT68276.1 | 13772 | 19129 | 165 | | | | | | | | |
| 219 | 19130 | 473 | CRT74835.1 | 13774 | 19131 | 165 | | | | | | | | |
| 220 | 19132 | 473 | CRT73989.1 | 13776 | 19133 | 165 | | | | | | | | |
| 221 | 19134 | 473 | CRT75035.1 | 13778 | 19135 | 165 | | | | | | | | |
| 222 | 19136 | 473 | CRT66762.1 | 13780 | 19137 | 165 | | | | | | | | |
| 223 | 19138 | 473 | CRT70566.1 | 13782 | 19139 | 165 | | | | | | | | |
| 224 | 19140 | 473 | CRT64116.1 | 13784 | 19141 | 165 | | | | | | | | |
| 225 | 19142 | 473 | CRT57991.1 | 13786 | 19143 | 165 | | | | | | | | |
| 226 | 19144 | 473 | CRT66324.1 | 13788 | 19145 | 165 | | | | | | | | |
| 227 | 19146 | 473 | CRT75784.1 | 13790 | 19147 | 165 | | | | | | | | |
| 228 | 19148 | 473 | CRT69390.1 | 13792 | 19149 | 165 | | | | | | | | |
| 229 | 19150 | 473 | CRT79545.1 | 13794 | 19151 | 165 | | | | | | | | |
| 230 | 19152 | 473 | CRT65368.1 | 13796 | 19153 | 165 | | | | | | | | |
| 231 | 19154 | 473 | CRT80471.1 | 13798 | 19155 | 165 | | | | | | | | |
| 232 | 19156 | 473 | CRT86536.1 | 13800 | 19157 | 165 | | | | | | | | |
| 233 | 19158 | 473 | CRT83015.1 | 13802 | 19159 | 165 | | | | | | | | |
| 234 | 19160 | 473 | CRT86807.1 | 13804 | 19161 | 165 | | | | | | | | |
| 235 | 19162 | 473 | CRT85049.1 | 13806 | 19163 | 165 | | | | | | | | |
| 236 | 19164 | 473 | CRT84210.1 | 13808 | 19165 | 165 | | | | | | | | |
| 237 | 19166 | 473 | CRT81912.1 | 13810 | 19167 | 165 | | | | | | | | |
| 238 | 19168 | 473 | CRT86929.1 | 13812 | 19169 | 165 | | | | | | | | |
| 239 | 19170 | 473 | CRT82839.1 | 13814 | 19171 | 165 | | | | | | | | |
| 240 | 19172 | 473 | CRT86340.1 | 13816 | 19173 | 165 | | | | | | | | |
| 241 | 19174 | 473 | CRT89320.1 | 13818 | 19175 | 165 | | | | | | | | |
| 242 | 19176 | 473 | CRT87538.1 | 13820 | 19177 | 165 | | | | | | | | |
| 243 | 19178 | 473 | CRT90535.1 | 13822 | 19179 | 165 | | | | | | | | |
| 244 | 19180 | 473 | CRT91762.1 | 13824 | 19181 | 165 | | | | | | | | |
| 245 | 19182 | 473 | CRU00010.1 | 13826 | 19183 | 165 | | | | | | | | |
| 246 | 19184 | 473 | CRT98481.1 | 13828 | 19185 | 165 | | | | | | | | |
| 247 | 19186 | 473 | CRT99304.1 | 13830 | 19187 | 165 | | | | | | | | |
| 248 | 19188 | 473 | CRT99602.1 | 13832 | 19189 | 165 | | | | | | | | |
| 249 | 19190 | 473 | CRU04442.1 | 13834 | 19191 | 165 | | | | | | | | |
| 250 | 19192 | 473 | CRU03264.1 | 13836 | 19193 | 165 | | | | | | | | |
| 251 | 19194 | 473 | CRU09870.1 | 13838 | 19195 | 165 | | | | | | | | |
| 252 | 19196 | 473 | CRU07131.1 | 13840 | 19197 | 165 | | | | | | | | |
| 253 | 19198 | 473 | CRU20227.1 | 13842 | 19199 | 165 | | | | | | | | |
| 254 | 19200 | 473 | CRU18225.1 | 13844 | 19201 | 165 | | | | | | | | |
| 255 | 19202 | 473 | CRU22361.1 | 13846 | 19203 | 165 | | | | | | | | |
| 256 | 19204 | 473 | CRU18030.1 | 13848 | 19205 | 165 | | | | | | | | |
| 257 | 19206 | 473 | CRU21809.1 | 13850 | 19207 | 165 | | | | | | | | |
| 258 | 19208 | 473 | CRU20299.1 | 13852 | 19209 | 165 | | | | | | | | |
| 259 | 19210 | 473 | CRU20889.1 | 13854 | 19211 | 165 | | | | | | | | |
| 260 | 19212 | 473 | CRU31581.1 | 13856 | 19213 | 165 | | | | | | | | |
| 261 | 19214 | 473 | CRU58956.1 | 13858 | 19215 | 165 | | | | | | | | |
| 262 | 19216 | 473 | CRU85953.1 | 13860 | 19217 | 165 | | | | | | | | |
| 263 | 19218 | 473 | CRU70039.1 | 13862 | 19219 | 165 | | | | | | | | |
| 264 | 19220 | 473 | CRU80230.1 | 13864 | 19221 | 165 | | | | | | | | |
| 265 | 19222 | 473 | CRU71479.1 | 13866 | 19223 | 165 | | | | | | | | |
| 266 | 19224 | 473 | CRU70694.1 | 13868 | 19225 | 165 | | | | | | | | |
| 267 | 19226 | 473 | CRU61514.1 | 13870 | 19227 | 165 | | | | | | | | |
| 268 | 19228 | 473 | CRU48049.1 | 13872 | 19229 | 165 | | | | | | | | |
| 269 | 19230 | 473 | CRU68137.1 | 13874 | 19231 | 165 | | | | | | | | |
| 270 | 19232 | 473 | CRU71318.1 | 13876 | 19233 | 165 | | | | | | | | |
| 271 | 19234 | 473 | CRU93815.1 | 13878 | 19235 | 165 | | | | | | | | |
| 272 | 19236 | 473 | CRU91179.1 | 13880 | 19237 | 165 | | | | | | | | |
| 273 | 19238 | 473 | CRU91126.1 | 13882 | 19239 | 165 | | | | | | | | |
| 274 | 19240 | 473 | CRU94296.1 | 13884 | 19241 | 165 | | | | | | | | |
| 275 | 19242 | 473 | CRU82319.1 | 13886 | 19243 | 165 | | | | | | | | |
| 276 | 19244 | 473 | CRU80792.1 | 13888 | 19245 | 165 | | | | | | | | |
| 277 | 19246 | 473 | CRU91423.1 | 13890 | 19247 | 165 | | | | | | | | |
| 278 | 19248 | 473 | CRU86616.1 | 13892 | 19249 | 165 | | | | | | | | |
| 279 | 19250 | 473 | CRU82587.1 | 13894 | 19251 | 165 | | | | | | | | |
| 280 | 19252 | 473 | CRU96749.1 | 13896 | 19253 | 165 | | | | | | | | |
| 281 | 19254 | 473 | CRU83071.1 | 13898 | 19255 | 165 | | | | | | | | |
| 282 | 19256 | 473 | CRU81012.1 | 13900 | 19257 | 165 | | | | | | | | |
| 283 | 19258 | 473 | CRU78269.1 | 13902 | 19259 | 165 | | | | | | | | |
| 284 | 19260 | 473 | CRV00482.1 | 13904 | 19261 | 165 | | | | | | | | |
| 285 | 19262 | 473 | CRV01086.1 | 13906 | 19263 | 165 | | | | | | | | |
| 286 | 19264 | 473 | CRU80178.1 | 13908 | 19265 | 165 | | | | | | | | |
| 287 | 19266 | 473 | CRU93089.1 | 13910 | 19267 | 165 | | | | | | | | |
| 288 | 19268 | 473 | CRU95681.1 | 13912 | 19269 | 165 | | | | | | | | |
| 289 | 19270 | 473 | CRU99739.1 | 13914 | 19271 | 165 | | | | | | | | |
| 290 | 19272 | 473 | CRV05003.1 | 13916 | 19273 | 165 | | | | | | | | |
| 291 | 19274 | 473 | CRU97011.1 | 13918 | 19275 | 165 | | | | | | | | |
| 292 | 19276 | 473 | CRV01718.1 | 13920 | 19277 | 165 | | | | | | | | |
| 293 | 19278 | 473 | CRV11884.1 | 13922 | 19279 | 165 | | | | | | | | |
| 294 | 19280 | 473 | CRV20258.1 | 13924 | 19281 | 165 | | | | | | | | |
| 295 | 19282 | 473 | CRV16508.1 | 13926 | 19283 | 165 | | | | | | | | |
| 296 | 19284 | 473 | CRV16547.1 | 13928 | 19285 | 165 | | | | | | | | |
| 297 | 19286 | 473 | CRV08852.1 | 13930 | 19287 | 165 | | | | | | | | |
| 298 | 19288 | 473 | CRV21821.1 | 13932 | 19289 | 165 | | | | | | | | |
| 299 | 19290 | 473 | CRV22393.1 | 13934 | 19291 | 165 | | | | | | | | |
| 300 | 19292 | 473 | CRV15620.1 | 13936 | 19293 | 165 | | | | | | | | |
| 301 | 19294 | 473 | CRV21794.1 | 13938 | 19295 | 165 | | | | | | | | |
| 302 | 19296 | 473 | CRV18929.1 | 13940 | 19297 | 165 | | | | | | | | |
| 303 | 19298 | 473 | CRV22815.1 | 13942 | 19299 | 165 | | | | | | | | |
| 304 | 19300 | 473 | CRV26122.1 | 13944 | 19301 | 165 | | | | | | | | |
| 305 | 19302 | 473 | CRV22957.1 | 13946 | 19303 | 165 | | | | | | | | |
| 306 | 19304 | 473 | CRV22952.1 | 13948 | 19305 | 165 | | | | | | | | |
| 307 | 19306 | 473 | CRV23373.1 | 13950 | 19307 | 165 | | | | | | | | |
| 308 | 19308 | 473 | CRV25540.1 | 13952 | 19309 | 165 | | | | | | | | |
| 309 | 19310 | 473 | CRV23993.1 | 13954 | 19311 | 165 | | | | | | | | |
| 310 | 19312 | 473 | CRV35423.1 | 13956 | 19313 | 165 | | | | | | | | |
| 311 | 19314 | 473 | CRV28499.1 | 13958 | 19315 | 165 | | | | | | | | |
| 312 | 19316 | 473 | CRV30748.1 | 13960 | 19317 | 165 | | | | | | | | |
| 313 | 19318 | 473 | CRV37966.1 | 13962 | 19319 | 165 | | | | | | | | |
| 314 | 19320 | 473 | CRV37006.1 | 13964 | 19321 | 165 | | | | | | | | |
| 315 | 19322 | 473 | CRV38821.1 | 13966 | 19323 | 165 | | | | | | | | |
| 316 | 19324 | 473 | CRV39783.1 | 13968 | 19325 | 165 | | | | | | | | |
| 317 | 19326 | 473 | CRV45918.1 | 13970 | 19327 | 165 | | | | | | | | |
| 318 | 19328 | 473 | CRV48895.1 | 13972 | 19329 | 165 | | | | | | | | |
| 319 | 19330 | 473 | CRV53129.1 | 13974 | 19331 | 165 | | | | | | | | |
| 320 | 19332 | 473 | CRV61825.1 | 13976 | 19333 | 165 | | | | | | | | |
| 321 | 19334 | 473 | CRV60050.1 | 13978 | 19335 | 165 | | | | | | | | |
| 322 | 19336 | 473 | CRV55444.1 | 13980 | 19337 | 165 | | | | | | | | |
| 323 | 19338 | 473 | CRV60789.1 | 13982 | 19339 | 165 | | | | | | | | |
| 324 | 19340 | 473 | CRW03939.1 | 13984 | 19341 | 165 | | | | | | | | |
| 325 | 19342 | 473 | CRV54218.1 | 13986 | 19343 | 165 | | | | | | | | |
| 326 | 19344 | 473 | CRV83250.1 | 13988 | 19345 | 165 | | | | | | | | |
| 327 | 19346 | 473 | CRV91433.1 | 13990 | 19347 | 165 | | | | | | | | |
| 328 | 19348 | 473 | CRV92210.1 | 13992 | 19349 | 165 | | | | | | | | |
| 329 | 19350 | 473 | CRV87155.1 | 13994 | 19351 | 165 | | | | | | | | |
| 330 | 19352 | 473 | CRV84642.1 | 13996 | 19353 | 165 | | | | | | | | |
| 331 | 19354 | 473 | CRW02978.1 | 13998 | 19355 | 165 | | | | | | | | |
| 332 | 19356 | 473 | CRV97172.1 | 14000 | 19357 | 165 | | | | | | | | |
| 333 | 19358 | 473 | CRW00512.1 | 14002 | 19359 | 165 | | | | | | | | |
| 334 | 19360 | 473 | CRW01306.1 | 14004 | 19361 | 165 | | | | | | | | |
| 335 | 19362 | 473 | CRV92070.1 | 14006 | 19363 | 165 | | | | | | | | |
| 336 | 19364 | 473 | CRV95683.1 | 14008 | 19365 | 165 | | | | | | | | |
| 337 | 19366 | 473 | CRV97960.1 | 14010 | 19367 | 165 | | | | | | | | |
| 338 | 19368 | 473 | CRV78510.1 | 14012 | 19369 | 165 | | | | | | | | |
| 339 | 19370 | 473 | CRW03677.1 | 14014 | 19371 | 165 | | | | | | | | |
| 340 | 19372 | 473 | CRV92174.1 | 14016 | 19373 | 165 | | | | | | | | |
| 341 | 19374 | 473 | CRV95859.1 | 14018 | 19375 | 165 | | | | | | | | |
| 342 | 19376 | 473 | CRV90213.1 | 14020 | 19377 | 165 | | | | | | | | |
| 343 | 19378 | 473 | CRW02419.1 | 14022 | 19379 | 165 | | | | | | | | |
| 344 | 19380 | 473 | CRW06354.1 | 14024 | 19381 | 165 | | | | | | | | |
| 345 | 19382 | 473 | CRW10299.1 | 14026 | 19383 | 165 | | | | | | | | |
| 346 | 19384 | 473 | CRV93338.1 | 14028 | 19385 | 165 | | | | | | | | |
| 347 | 19386 | 473 | CRW10494.1 | 14030 | 19387 | 165 | | | | | | | | |
| 348 | 19388 | 473 | CRW11905.1 | 14032 | 19389 | 165 | | | | | | | | |
| 349 | 19390 | 473 | CRW12150.1 | 14034 | 19391 | 165 | | | | | | | | |
| 350 | 19392 | 473 | CRW18044.1 | 14036 | 19393 | 165 | | | | | | | | |
| 351 | 19394 | 473 | CRW22911.1 | 14038 | 19395 | 165 | | | | | | | | |
| 352 | 19396 | 473 | CRW16832.1 | 14040 | 19397 | 165 | | | | | | | | |
| 353 | 19398 | 473 | CRW17385.1 | 14042 | 19399 | 165 | | | | | | | | |
| 354 | 19400 | 473 | CRW41624.1 | 14044 | 19401 | 165 | | | | | | | | |
| 355 | 19402 | 473 | CRW33363.1 | 14046 | 19403 | 165 | | | | | | | | |
| 356 | 19404 | 473 | CRW28608.1 | 14048 | 19405 | 165 | | | | | | | | |
| 357 | 19406 | 473 | CRW38422.1 | 14050 | 19407 | 165 | | | | | | | | |
| 358 | 19408 | 473 | CRW56682.1 | 14052 | 19409 | 165 | | | | | | | | |
| 359 | 19410 | 473 | CRW57506.1 | 14054 | 19411 | 165 | | | | | | | | |
| 360 | 19412 | 473 | CRW51171.1 | 14056 | 19413 | 165 | | | | | | | | |
| 361 | 19414 | 473 | CRW56053.1 | 14058 | 19415 | 165 | | | | | | | | |
| 362 | 19416 | 473 | CRW51028.1 | 14060 | 19417 | 165 | | | | | | | | |
| 363 | 19418 | 473 | CRW62438.1 | 14062 | 19419 | 165 | | | | | | | | |
| 364 | 19420 | 473 | CRW52653.1 | 14064 | 19421 | 165 | | | | | | | | |
| 365 | 19422 | 473 | CRW57935.1 | 14066 | 19423 | 165 | | | | | | | | |
| 366 | 19424 | 473 | CRW58504.1 | 14068 | 19425 | 165 | | | | | | | | |
| 367 | 19426 | 473 | CRW59077.1 | 14070 | 19427 | 165 | | | | | | | | |
| 368 | 19428 | 473 | CRW78808.1 | 14072 | 19429 | 165 | | | | | | | | |
| 369 | 19430 | 473 | CRW72028.1 | 14074 | 19431 | 165 | | | | | | | | |
| 370 | 19432 | 473 | CRW79987.1 | 14076 | 19433 | 165 | | | | | | | | |
| 371 | 19434 | 473 | CRW76327.1 | 14078 | 19435 | 165 | | | | | | | | |
| 372 | 19436 | 473 | CRW80128.1 | 14080 | 19437 | 165 | | | | | | | | |
| 373 | 19438 | 473 | CRW66999.1 | 14082 | 19439 | 165 | | | | | | | | |
| 374 | 19440 | 473 | CRW70540.1 | 14084 | 19441 | 165 | | | | | | | | |
| 375 | 19442 | 473 | CRW76747.1 | 14086 | 19443 | 165 | | | | | | | | |
| 376 | 19444 | 473 | CRW76878.1 | 14088 | 19445 | 165 | | | | | | | | |
| 377 | 19446 | 473 | CRW80962.1 | 14090 | 19447 | 165 | | | | | | | | |
| 378 | 19448 | 473 | CRW81686.1 | 14092 | 19449 | 165 | | | | | | | | |
| 379 | 19450 | 473 | CRW77668.1 | 14094 | 19451 | 165 | | | | | | | | |
| 380 | 19452 | 473 | CRW79793.1 | 14096 | 19453 | 165 | | | | | | | | |
| 381 | 19454 | 473 | CRW80957.1 | 14098 | 19455 | 165 | | | | | | | | |
| 382 | 19456 | 481 | CTT29891.1 | 14100 | 19457 | 155 | | | | | | | | |
| 383 | 19458 | 481 | CTZ05317.1 | 14102 | 19459 | 155 | | | | | | | | |
| 384 | 19460 | 491 | CUB16901.1 | 14104 | 19461 | 165 | | | | | | | | |
| 385 | 19462 | 482 | CUI41016.1 | 14106 | 19463 | 161 | CUI41048.1 | 14107 | 19464 | 193 | CUI41311.1 | 14108 | 19465 | 155 |
| 386 | 19467 | 473 | CUK22711.1 | 14111 | 19468 | 146 | CUK22702.1 | 14112 | 19469 | 113 | | | | |
| 387 | 19470 | 486 | CUK50737.1 | 14114 | 19471 | 202 | | | | | | | | |
| 388 | 19472 | 486 | CUK61278.1 | 14116 | 19473 | 202 | | | | | | | | |
| 389 | 19474 | 486 | CUK91822.1 | 14118 | 19475 | 202 | | | | | | | | |
| 390 | 19476 | 461 | EDM51054.1 | 14120 | 19477 | 203 | EDM51056.1 | 14121 | 19478 | 188 | EDM51058.1 | 14122 | 19479 | 282 |
| 391 | 19480 | 484 | CUY08443.1 | 14124 | 19481 | 163 | | | | | | | | |
| 392 | 19482 | 484 | CUZ68596.1 | 14126 | 19483 | 163 | | | | | | | | |
| 393 | 19484 | 484 | CUZ82333.1 | 14128 | 19485 | 163 | | | | | | | | |
| 394 | 19486 | 484 | CUZ53179.1 | 14130 | 19487 | 163 | | | | | | | | |
| 395 | 19488 | 484 | CVA27418.1 | 14132 | 19489 | 163 | | | | | | | | |
| 396 | 19490 | 484 | CUZ41313.1 | 14134 | 19491 | 163 | | | | | | | | |
| 397 | 19492 | 484 | CVA08079.1 | 14136 | 19493 | 163 | | | | | | | | |
| 398 | 19494 | 484 | CUZ42337.1 | 14138 | 19495 | 163 | | | | | | | | |
| 399 | 19496 | 484 | CVA28833.1 | 14140 | 19497 | 163 | | | | | | | | |
| 400 | 19498 | 484 | CUZ16090.1 | 14142 | 19499 | 163 | | | | | | | | |
| 401 | 19500 | 484 | CUZ61026.1 | 14144 | 19501 | 163 | | | | | | | | |
| 402 | 19502 | 484 | CUZ90983.1 | 14146 | 19503 | 163 | | | | | | | | |
| 403 | 19504 | 484 | CUZ73356.1 | 14148 | 19505 | 163 | | | | | | | | |
| 404 | 19506 | 484 | CVA13520.1 | 14150 | 19507 | 163 | | | | | | | | |
| 405 | 19508 | 484 | CVA22984.1 | 14152 | 19509 | 163 | | | | | | | | |
| 406 | 19510 | 484 | CUY16507.1 | 14154 | 19511 | 163 | | | | | | | | |
| 407 | 19512 | 484 | CUZ58687.1 | 14156 | 19513 | 163 | | | | | | | | |
| 408 | 19514 | 484 | CUZ73597.1 | 14158 | 19515 | 163 | | | | | | | | |
| 409 | 19516 | 484 | CUZ84806.1 | 14160 | 19517 | 163 | | | | | | | | |
| 410 | 19518 | 484 | CUZ51268.1 | 14162 | 19519 | 163 | | | | | | | | |
| 411 | 19520 | 484 | CVA06067.1 | 14164 | 19521 | 163 | | | | | | | | |
| 412 | 19522 | 484 | CUZ66217.1 | 14166 | 19523 | 163 | | | | | | | | |
| 413 | 19524 | 484 | CUZ96361.1 | 14168 | 19525 | 163 | | | | | | | | |
| 414 | 19526 | 484 | CUZ56063.1 | 14170 | 19527 | 163 | | | | | | | | |
| 415 | 19528 | 484 | CUZ47666.1 | 14172 | 19529 | 163 | | | | | | | | |
| 416 | 19530 | 306 | CUZ65482.1 | 14174 | 19531 | 163 | | | | | | | | |
| 417 | 19532 | 484 | CUZ64434.1 | 14176 | 19533 | 163 | | | | | | | | |
| 418 | 19534 | 484 | CUZ76056.1 | 14178 | 19535 | 163 | | | | | | | | |
| 419 | 19536 | 484 | CVA13481.1 | 14180 | 19537 | 163 | | | | | | | | |
| 420 | 19538 | 484 | CVA27120.1 | 14182 | 19539 | 163 | | | | | | | | |
| 421 | 19540 | 484 | CVA15846.1 | 14184 | 19541 | 163 | | | | | | | | |
| 422 | 19542 | 484 | CUZ46999.1 | 14186 | 19543 | 163 | | | | | | | | |
| 423 | 19544 | 484 | CUZ30986.1 | 14188 | 19545 | 163 | | | | | | | | |
| 424 | 19546 | 484 | CUZ02657.1 | 14190 | 19547 | 163 | | | | | | | | |
| 425 | 19548 | 484 | CUZ51348.1 | 14192 | 19549 | 163 | | | | | | | | |
| 426 | 19550 | 484 | CUZ55112.1 | 14194 | 19551 | 163 | | | | | | | | |
| 427 | 19552 | 484 | CVA20234.1 | 14196 | 19553 | 163 | | | | | | | | |
| 428 | 19554 | 484 | CUZ69722.1 | 14198 | 19555 | 163 | | | | | | | | |
| 429 | 19556 | 306 | CUZ82685.1 | 14200 | 19557 | 163 | | | | | | | | |
| 430 | 19558 | 484 | CUZ92253.1 | 14202 | 19559 | 163 | | | | | | | | |
| 431 | 19560 | 484 | CUZ80137.1 | 14204 | 19561 | 163 | | | | | | | | |
| 432 | 19562 | 306 | CUZ43160.1 | 14206 | 19563 | 163 | | | | | | | | |
| 433 | 19564 | 484 | CVA39205.1 | 14208 | 19565 | 163 | | | | | | | | |
| 434 | 19566 | 484 | CVA66680.1 | 14210 | 19567 | 163 | | | | | | | | |
| 435 | 19568 | 484 | CUZ78128.1 | 14212 | 19569 | 163 | | | | | | | | |
| 436 | 19570 | 484 | CUZ53811.1 | 14214 | 19571 | 163 | | | | | | | | |
| 437 | 19572 | 484 | CVA07157.1 | 14216 | 19573 | 163 | | | | | | | | |
| 438 | 19574 | 484 | CUZ96160.1 | 14218 | 19575 | 163 | | | | | | | | |
| 439 | 19576 | 484 | CVA63355.1 | 14220 | 19577 | 163 | | | | | | | | |
| 440 | 19578 | 484 | CVB19765.1 | 14222 | 19579 | 163 | | | | | | | | |
| 441 | 19580 | 484 | CVB15307.1 | 14224 | 19581 | 163 | | | | | | | | |
| 442 | 19582 | 484 | CVB18610.1 | 14226 | 19583 | 163 | | | | | | | | |
| 443 | 19584 | 484 | CVC33373.1 | 14228 | 19585 | 163 | | | | | | | | |
| 444 | 19586 | 484 | CVC43459.1 | 14230 | 19587 | 163 | | | | | | | | |
| 445 | 19588 | 484 | CVB44154.1 | 14232 | 19589 | 163 | | | | | | | | |
| 446 | 19590 | 484 | CVC13577.1 | 14234 | 19591 | 163 | | | | | | | | |
| 447 | 19592 | 306 | CVB24202.1 | 14236 | 19593 | 163 | | | | | | | | |
| 448 | 19594 | 484 | CVB63328.1 | 14238 | 19595 | 163 | | | | | | | | |
| 449 | 19596 | 484 | CVB58992.1 | 14240 | 19597 | 163 | | | | | | | | |
| 450 | 19598 | 306 | CVB73390.1 | 14242 | 19599 | 163 | | | | | | | | |
| 451 | 19600 | 306 | CVA22542.1 | 14244 | 19601 | 163 | | | | | | | | |
| 452 | 19602 | 484 | CVC54883.1 | 14246 | 19603 | 163 | | | | | | | | |
| 453 | 19604 | 484 | CVC47662.1 | 14248 | 19605 | 163 | | | | | | | | |
| 454 | 19606 | 484 | CVC16868.1 | 14250 | 19607 | 163 | | | | | | | | |
| 455 | 19608 | 484 | CVA94551.1 | 14252 | 19609 | 163 | | | | | | | | |
| 456 | 19610 | 484 | CVB98061.1 | 14254 | 19611 | 163 | | | | | | | | |
| 457 | 19612 | 484 | CVB27563.1 | 14256 | 19613 | 163 | | | | | | | | |
| 458 | 19614 | 484 | CVC12710.1 | 14258 | 19615 | 163 | | | | | | | | |
| 459 | 19616 | 484 | CVB94236.1 | 14260 | 19617 | 163 | | | | | | | | |
| 460 | 19618 | 484 | CVC34514.1 | 14262 | 19619 | 163 | | | | | | | | |
| 461 | 19620 | 484 | CVC37237.1 | 14264 | 19621 | 163 | | | | | | | | |
| 462 | 19622 | 484 | CVC09959.1 | 14266 | 19623 | 163 | | | | | | | | |
| 463 | 19624 | 484 | CVB51982.1 | 14268 | 19625 | 163 | | | | | | | | |
| 464 | 19626 | 484 | CVB83683.1 | 14270 | 19627 | 163 | | | | | | | | |
| 465 | 19628 | 484 | CVB86104.1 | 14272 | 19629 | 163 | | | | | | | | |
| 466 | 19630 | 484 | CVC63999.1 | 14274 | 19631 | 163 | | | | | | | | |
| 467 | 19632 | 306 | CVB86034.1 | 14276 | 19633 | 163 | | | | | | | | |
| 468 | 19634 | 484 | CVC85541.1 | 14278 | 19635 | 163 | | | | | | | | |
| 469 | 19636 | 484 | CVC33606.1 | 14280 | 19637 | 163 | | | | | | | | |
| 470 | 19638 | 484 | CVC91020.1 | 14282 | 19639 | 163 | | | | | | | | |
| 471 | 19640 | 484 | CVC23340.1 | 14284 | 19641 | 163 | | | | | | | | |
| 472 | 19642 | 484 | CVC01465.1 | 14286 | 19643 | 163 | | | | | | | | |
| 473 | 19644 | 484 | CVC17896.1 | 14288 | 19645 | 163 | | | | | | | | |
| 474 | 19646 | 484 | CVC49253.1 | 14290 | 19647 | 163 | | | | | | | | |
| 475 | 19648 | 306 | CVD13477.1 | 14292 | 19649 | 163 | | | | | | | | |
| 476 | 19650 | 484 | CVC16333.1 | 14294 | 19651 | 163 | | | | | | | | |
| 477 | 19652 | 484 | CVC38388.1 | 14296 | 19653 | 163 | | | | | | | | |
| 478 | 19654 | 484 | CVC81405.1 | 14298 | 19655 | 163 | | | | | | | | |
| 479 | 19656 | 484 | CVC81576.1 | 14300 | 19657 | 163 | | | | | | | | |
| 480 | 19658 | 484 | CVC86607.1 | 14302 | 19659 | 163 | | | | | | | | |
| 481 | 19660 | 484 | CVC98763.1 | 14304 | 19661 | 163 | | | | | | | | |
| 482 | 19662 | 484 | CVC91159.1 | 14306 | 19663 | 163 | | | | | | | | |
| 483 | 19664 | 484 | CVC94687.1 | 14308 | 19665 | 163 | | | | | | | | |
| 484 | 19666 | 484 | CVC52230.1 | 14310 | 19667 | 163 | | | | | | | | |
| 485 | 19668 | 484 | CVC98236.1 | 14312 | 19669 | 163 | | | | | | | | |
| 486 | 19670 | 484 | CVB21750.1 | 14314 | 19671 | 163 | | | | | | | | |
| 487 | 19672 | 484 | CVC48200.1 | 14316 | 19673 | 163 | | | | | | | | |
| 488 | 19674 | 484 | CVC71583.1 | 14318 | 19675 | 163 | | | | | | | | |
| 489 | 19676 | 484 | CVC84184.1 | 14320 | 19677 | 163 | | | | | | | | |
| 490 | 19678 | 306 | CVC59774.1 | 14322 | 19679 | 163 | | | | | | | | |
| 491 | 19680 | 484 | CVE09079.1 | 14324 | 19681 | 163 | | | | | | | | |
| 492 | 19682 | 484 | CVD69880.1 | 14326 | 19683 | 163 | | | | | | | | |
| 493 | 19684 | 484 | CVD52024.1 | 14328 | 19685 | 163 | | | | | | | | |
| 494 | 19686 | 484 | CVD94582.1 | 14330 | 19687 | 163 | | | | | | | | |
| 495 | 19688 | 484 | CVD41050.1 | 14332 | 19689 | 163 | | | | | | | | |
| 496 | 19690 | 306 | CVD28932.1 | 14334 | 19691 | 163 | | | | | | | | |
| 497 | 19692 | 484 | CVC37080.1 | 14336 | 19693 | 163 | | | | | | | | |
| 498 | 19694 | 484 | CVE33508.1 | 14338 | 19695 | 163 | | | | | | | | |
| 499 | 19696 | 306 | CVC40354.1 | 14340 | 19697 | 163 | | | | | | | | |
| 500 | 19698 | 306 | CVE27819.1 | 14342 | 19699 | 163 | | | | | | | | |
| 501 | 19700 | 484 | CVE60700.1 | 14344 | 19701 | 163 | | | | | | | | |
| 502 | 19702 | 306 | CVE69177.1 | 14346 | 19703 | 163 | | | | | | | | |
| 503 | 19704 | 484 | CVE06156.1 | 14348 | 19705 | 163 | | | | | | | | |
| 504 | 19706 | 484 | CVE70674.1 | 14350 | 19707 | 163 | | | | | | | | |
| 505 | 19708 | 484 | CVD68135.1 | 14352 | 19709 | 163 | | | | | | | | |
| 506 | 19710 | 484 | CVE19607.1 | 14354 | 19711 | 163 | | | | | | | | |
| 507 | 19712 | 484 | CVE94911.1 | 14356 | 19713 | 163 | | | | | | | | |
| 508 | 19714 | 484 | CVE74015.1 | 14358 | 19715 | 163 | | | | | | | | |
| 509 | 19716 | 484 | CVE69277.1 | 14360 | 19717 | 163 | | | | | | | | |
| 510 | 19718 | 484 | CVD84064.1 | 14362 | 19719 | 163 | | | | | | | | |
| 511 | 19720 | 484 | CVE29091.1 | 14364 | 19721 | 163 | | | | | | | | |
| 512 | 19722 | 484 | CVD89467.1 | 14366 | 19723 | 163 | | | | | | | | |
| 513 | 19724 | 306 | CVE62900.1 | 14368 | 19725 | 163 | | | | | | | | |
| 514 | 19726 | 484 | CVE95691.1 | 14370 | 19727 | 163 | | | | | | | | |
| 515 | 19728 | 484 | CVF11375.1 | 14372 | 19729 | 163 | | | | | | | | |
| 516 | 19730 | 484 | CVF28649.1 | 14374 | 19731 | 163 | | | | | | | | |
| 517 | 19732 | 484 | CVC97390.1 | 14376 | 19733 | 163 | | | | | | | | |
| 518 | 19734 | 484 | CVD52643.1 | 14378 | 19735 | 163 | | | | | | | | |
| 519 | 19736 | 484 | CVE22758.1 | 14380 | 19737 | 163 | | | | | | | | |
| 520 | 19738 | 484 | CVE97454.1 | 14382 | 19739 | 163 | | | | | | | | |
| 521 | 19740 | 484 | CVF28158.1 | 14384 | 19741 | 163 | | | | | | | | |
| 522 | 19742 | 484 | CVE60533.1 | 14386 | 19743 | 163 | | | | | | | | |
| 523 | 19744 | 484 | CVE66058.1 | 14388 | 19745 | 163 | | | | | | | | |
| 524 | 19746 | 484 | CVF01477.1 | 14390 | 19747 | 163 | | | | | | | | |
| 525 | 19748 | 484 | CVE93253.1 | 14392 | 19749 | 163 | | | | | | | | |
| 526 | 19750 | 484 | CVF50134.1 | 14394 | 19751 | 163 | | | | | | | | |
| 527 | 19752 | 306 | CVF09910.1 | 14396 | 19753 | 163 | | | | | | | | |
| 528 | 19754 | 484 | CVE63411.1 | 14398 | 19755 | 163 | | | | | | | | |
| 529 | 19756 | 484 | CVE05444.1 | 14400 | 19757 | 163 | | | | | | | | |
| 530 | 19758 | 484 | CVE67662.1 | 14402 | 19759 | 163 | | | | | | | | |
| 531 | 19760 | 484 | CVE73566.1 | 14404 | 19761 | 163 | | | | | | | | |
| 532 | 19762 | 484 | CVF13064.1 | 14406 | 19763 | 163 | | | | | | | | |
| 533 | 19764 | 484 | CVE92994.1 | 14408 | 19765 | 163 | | | | | | | | |
| 534 | 19766 | 484 | CVF15667.1 | 14410 | 19767 | 163 | | | | | | | | |
| 535 | 19768 | 484 | CVE70361.1 | 14412 | 19769 | 163 | | | | | | | | |
| 536 | 19770 | 484 | CVF77810.1 | 14414 | 19771 | 163 | | | | | | | | |
| 537 | 19772 | 484 | CVF08046.1 | 14416 | 19773 | 163 | | | | | | | | |
| 538 | 19774 | 484 | CVF84133.1 | 14418 | 19775 | 163 | | | | | | | | |
| 539 | 19776 | 484 | CVF82137.1 | 14420 | 19777 | 163 | | | | | | | | |
| 540 | 19778 | 484 | CVG26701.1 | 14422 | 19779 | 163 | | | | | | | | |
| 541 | 19780 | 484 | CVG63887.1 | 14424 | 19781 | 163 | | | | | | | | |
| 542 | 19782 | 484 | CVG49433.1 | 14426 | 19783 | 163 | | | | | | | | |
| 543 | 19784 | 484 | CVF96091.1 | 14428 | 19785 | 163 | | | | | | | | |
| 544 | 19786 | 484 | CVG60811.1 | 14430 | 19787 | 163 | | | | | | | | |
| 545 | 19788 | 484 | CVG18576.1 | 14432 | 19789 | 163 | | | | | | | | |
| 546 | 19790 | 484 | CVG60263.1 | 14434 | 19791 | 163 | | | | | | | | |
| 547 | 19792 | 484 | CVG00271.1 | 14436 | 19793 | 163 | | | | | | | | |
| 548 | 19794 | 484 | CVG18115.1 | 14438 | 19795 | 163 | | | | | | | | |
| 549 | 19796 | 484 | CVG66394.1 | 14440 | 19797 | 163 | | | | | | | | |
| 550 | 19798 | 306 | CVF82722.1 | 14442 | 19799 | 163 | | | | | | | | |
| 551 | 19800 | 484 | CVG27190.1 | 14444 | 19801 | 163 | | | | | | | | |
| 552 | 19802 | 484 | CVH08322.1 | 14446 | 19803 | 163 | | | | | | | | |
| 553 | 19804 | 306 | CVG82345.1 | 14448 | 19805 | 163 | | | | | | | | |
| 554 | 19806 | 484 | CVG89971.1 | 14450 | 19807 | 163 | | | | | | | | |
| 555 | 19808 | 484 | CVG75193.1 | 14452 | 19809 | 163 | | | | | | | | |
| 556 | 19810 | 484 | CVG91902.1 | 14454 | 19811 | 163 | | | | | | | | |
| 557 | 19812 | 484 | CVG61740.1 | 14456 | 19813 | 163 | | | | | | | | |
| 558 | 19814 | 484 | CVG55691.1 | 14458 | 19815 | 163 | | | | | | | | |
| 559 | 19816 | 484 | CVG99595.1 | 14460 | 19817 | 163 | | | | | | | | |
| 560 | 19818 | 484 | CVG85863.1 | 14462 | 19819 | 163 | | | | | | | | |
| 561 | 19820 | 484 | CVH13264.1 | 14464 | 19821 | 163 | | | | | | | | |
| 562 | 19822 | 484 | CVH22072.1 | 14466 | 19823 | 163 | | | | | | | | |
| 563 | 19824 | 484 | CVG79637.1 | 14468 | 19825 | 163 | | | | | | | | |
| 564 | 19826 | 484 | CVH31713.1 | 14470 | 19827 | 163 | | | | | | | | |
| 565 | 19828 | 484 | CVG61487.1 | 14472 | 19829 | 163 | | | | | | | | |
| 566 | 19830 | 484 | CVF47762.1 | 14474 | 19831 | 163 | | | | | | | | |
| 567 | 19832 | 484 | CVH23246.1 | 14476 | 19833 | 163 | | | | | | | | |
| 568 | 19834 | 484 | CVG86968.1 | 14478 | 19835 | 163 | | | | | | | | |
| 569 | 19836 | 484 | CVG92546.1 | 14480 | 19837 | 163 | | | | | | | | |
| 570 | 19838 | 484 | CVH34063.1 | 14482 | 19839 | 163 | | | | | | | | |
| 571 | 19840 | 465 | CVG73755.1 | 14484 | 19841 | 163 | | | | | | | | |
| 572 | 19842 | 484 | CVH31045.1 | 14486 | 19843 | 163 | | | | | | | | |
| 573 | 19844 | 484 | CVH30700.1 | 14488 | 19845 | 163 | | | | | | | | |
| 574 | 19846 | 484 | CVG97923.1 | 14490 | 19847 | 163 | | | | | | | | |
| 575 | 19848 | 484 | CVH42939.1 | 14492 | 19849 | 163 | | | | | | | | |
| 576 | 19850 | 484 | CVH11711.1 | 14494 | 19851 | 163 | | | | | | | | |
| 577 | 19852 | 484 | CVH39720.1 | 14496 | 19853 | 163 | | | | | | | | |
| 578 | 19854 | 484 | CVH33846.1 | 14498 | 19855 | 163 | | | | | | | | |
| 579 | 19856 | 484 | CVG91297.1 | 14500 | 19857 | 163 | | | | | | | | |
| 580 | 19858 | 484 | CVH37749.1 | 14502 | 19859 | 163 | | | | | | | | |
| 581 | 19860 | 484 | CVH21011.1 | 14504 | 19861 | 163 | | | | | | | | |
| 582 | 19862 | 484 | CVG01680.1 | 14506 | 19863 | 163 | | | | | | | | |
| 583 | 19864 | 484 | CVH35255.1 | 14508 | 19865 | 163 | | | | | | | | |
| 584 | 19866 | 484 | CVH07271.1 | 14510 | 19867 | 163 | | | | | | | | |
| 585 | 19868 | 484 | CVH30938.1 | 14512 | 19869 | 163 | | | | | | | | |
| 586 | 19870 | 484 | CVH50697.1 | 14514 | 19871 | 163 | | | | | | | | |
| 587 | 19872 | 484 | CVH40526.1 | 14516 | 19873 | 163 | | | | | | | | |
| 588 | 19874 | 484 | CVH64630.1 | 14518 | 19875 | 163 | | | | | | | | |
| 589 | 19876 | 484 | CVH70543.1 | 14520 | 19877 | 163 | | | | | | | | |
| 590 | 19878 | 484 | CVH72213.1 | 14522 | 19879 | 163 | | | | | | | | |
| 591 | 19880 | 484 | CVH65593.1 | 14524 | 19881 | 163 | | | | | | | | |
| 592 | 19882 | 593 | CVJ13862.1 | 14526 | 19883 | 505 | | | | | | | | |
| 593 | 19884 | 479 | CVJ11186.1 | 14528 | 19885 | 166 | CVJ11217.1 | 14529 | 19886 | 257 | | | | |
| 594 | 19887 | 335 | CVJ34976.1 | 14531 | 19888 | 293 | | | | | | | | |
| 595 | 19889 | 486 | CWV39708.1 | 14533 | 19890 | 202 | | | | | | | | |
| 596 | 19891 | 486 | CWW37521.1 | 14535 | 19892 | 202 | | | | | | | | |
| 597 | 19893 | 469 | CYO51346.1 | 14537 | 19894 | 206 | CYO51297.1 | 14538 | 19895 | 388 | | | | |
| 598 | 19896 | 468 | CYT98226.1 | 14540 | 19897 | 156 | | | | | | | | |
| 599 | 19898 | 469 | CZB11023.1 | 14542 | 19899 | 206 | CZB10962.1 | 14543 | 19900 | 382 | | | | |
| 600 | 19901 | 469 | CZB35760.1 | 14545 | 19902 | 206 | CZB35774.1 | 14546 | 19903 | 382 | | | | |
| 601 | 19904 | 125 | CZD24096.1 | 14548 | 19905 | 184 | | | | | | | | |
| 602 | 19906 | 125 | CZC77665.1 | 14550 | 19907 | 184 | | | | | | | | |
| 603 | 19908 | 125 | CZD72514.1 | 14552 | 19909 | 184 | | | | | | | | |
| 604 | 19910 | 125 | CZE23710.1 | 14554 | 19911 | 184 | | | | | | | | |
| 605 | 19912 | 473 | CAW93166.1 | 14556 | 19913 | 165 | | | | | | | | |
| 606 | 19914 | 345 | CBK94113.1 | 14558 | 19915 | 260 | | | | | | | | |
| 607 | 19916 | 483 | CCE59806.1 | 14560 | 19917 | 202 | CCE59807.1 | 14561 | 19918 | 362 | | | | |
| 608 | 19919 | 472 | CDD80087.1 | 14563 | 19920 | 166 | CDD80084.1 | 14564 | 19921 | 196 | | | | |
| 609 | 19922 | 472 | EEG72545.1 | 14566 | 19923 | 203 | | | | | | | | |
| 610 | 19924 | 424 | EEP27314.1 | 14568 | 19925 | 163 | EEP27315.1 | 14569 | 19926 | 189 | EEP27316.1 | 14570 | 19927 | 376 |
| 611 | 19928 | 475 | EEI83076.1 | 14572 | 19929 | 205 | | | | | | | | |
| 612 | 19930 | 478 | EEV44994.1 | 14574 | 19931 | 161 | EEV44996.1 | 14575 | 19932 | 283 | | | | |
| 613 | 19933 | 485 | EES71590.1 | 14577 | 19934 | 227 | | | | | | | | |
| 614 | 19935 | 471 | EEY94493.1 | 14579 | 19936 | 231 | EEY94495.1 | 14580 | 19937 | 194 | | | | |
| 615 | 19938 | 478 | EFS10289.1 | 14582 | 19939 | 161 | EFS10291.1 | 14583 | 19940 | 283 | | | | |
| 616 | 19941 | 478 | EGF19708.1 | 14585 | 19942 | 206 | EGF19710.1 | 14586 | 19943 | 388 | | | | |
| 617 | 19944 | 476 | EGK00062.1 | 14588 | 19945 | 203 | EGK00061.1 | 14589 | 19946 | 191 | | | | |
| 618 | 19947 | 1379 | CCH29380.1 | 14591 | 19948 | 404 | | | | | | | | |
| 619 | 19949 | 473 | CCN04568.1 | 14593 | 19950 | 146 | | | | | | | | |
| 620 | 19951 | 484 | CDG13495.1 | 14595 | 19952 | 144 | | | | | | | | |
| 621 | 19953 | 485 | CDH26047.1 | 14597 | 19954 | 196 | | | | | | | | |
| 622 | 19955 | 478 | EZP94705.1 | 14599 | 19956 | 161 | EZP94707.1 | 14600 | 19957 | 283 | | | | |
| 623 | 19958 | 478 | EZP99084.1 | 14602 | 19959 | 161 | EZP99086.1 | 14603 | 19960 | 283 | | | | |
| 624 | 19961 | 478 | EZP92277.1 | 14605 | 19962 | 161 | EZP92279.1 | 14606 | 19963 | 283 | | | | |
| 625 | 19964 | 479 | ETZ11574.1 | 14608 | 19965 | 163 | | | | | | | | |
| 626 | 19966 | 480 | KER34864.1 | 14610 | 19967 | 155 | | | | | | | | |
| 627 | 19968 | 245 | EWC94440.1 | 14612 | 19969 | 376 | | | | | | | | |
| 628 | 19970 | 503 | KYF75626.1 | 14614 | 19971 | 956 | | | | | | | | |
| 629 | 19972 | 1686 | KYF75481.1 | 14616 | 19973 | 953 | | | | | | | | |
| 630 | 19974 | 482 | EXE88612.1 | 14618 | 19975 | 199 | EXE88616.1 | 14619 | 19976 | 165 | EXE88611.1 | 14620 | 19977 | 166 |
| 631 | 19980 | 434 | KDN27531.1 | 14624 | 19981 | 271 | | | | | | | | |
| 632 | 19982 | 482 | KEC78310.1 | 14626 | 19983 | 158 | KEC78315.1 | 14627 | 19984 | 161 | | | | |
| 633 | 19985 | 473 | KDC94657.1 | 14629 | 19986 | 146 | | | | | | | | |
| 634 | 19987 | 476 | EGY80389.1 | 14631 | 19988 | 99 | | | | | | | | |
| 635 | 19989 | 508 | EHI41039.1 | 14633 | 19990 | 377 | | | | | | | | |
| 636 | 19991 | 487 | EHI61290.1 | 14635 | 19992 | 239 | | | | | | | | |
| 637 | 19993 | 481 | EHP50821.1 | 14637 | 19994 | 163 | EHP50818.1 | 14638 | 19995 | 181 | | | | |
| 638 | 19996 | 410 | EHR31701.1 | 14640 | 19997 | 165 | | | | | | | | |
| 639 | 19998 | 471 | EIY18767.1 | 14642 | 19999 | 203 | EIY18769.1 | 14643 | 20000 | 182 | EIY18765.1 | 14644 | 20001 | 398 |
| 640 | 20002 | 471 | EIY40459.1 | 14646 | 20003 | 203 | EIY40461.1 | 14647 | 20004 | 182 | EIY40505.1 | 14648 | 20005 | 398 |
| 641 | 20006 | 477 | EIY98047.1 | 14650 | 20007 | 163 | | | | | | | | |
| 642 | 20008 | 477 | EIY91471.1 | 14652 | 20009 | 163 | | | | | | | | |
| 643 | 20010 | 477 | EIY90207.1 | 14654 | 20011 | 163 | | | | | | | | |
| 644 | 20012 | 477 | EIY98755.1 | 14656 | 20013 | 163 | | | | | | | | |
| 645 | 20014 | 477 | EIY95126.1 | 14658 | 20015 | 163 | | | | | | | | |
| 646 | 20016 | 477 | EIY94596.1 | 14660 | 20017 | 163 | | | | | | | | |
| 647 | 20018 | 477 | EIY90858.1 | 14662 | 20019 | 163 | | | | | | | | |
| 648 | 20020 | 489 | EJS10771.1 | 14664 | 20021 | 164 | | | | | | | | |
| 649 | 20022 | 494 | EJS00270.1 | 14666 | 20023 | 202 | | | | | | | | |
| 650 | 20024 | 474 | EJR09241.1 | 14668 | 20025 | 192 | EJR09239.1 | 14669 | 20026 | 193 | | | | |
| 651 | 20027 | 480 | EJV74396.1 | 14671 | 20028 | 201 | | | | | | | | |
| 652 | 20029 | 476 | EJU93679.1 | 14673 | 20030 | 207 | EJU93678.1 | 14674 | 20031 | 308 | | | | |
| 653 | 20032 | 478 | EJX46904.1 | 14676 | 20033 | 161 | EJX46906.1 | 14677 | 20034 | 283 | | | | |
| 654 | 20035 | 478 | EJX62526.1 | 14679 | 20036 | 161 | EJX62524.1 | 14680 | 20037 | 283 | | | | |
| 655 | 20038 | 478 | EJX62293.1 | 14682 | 20039 | 161 | EJX62295.1 | 14683 | 20040 | 283 | | | | |
| 656 | 20041 | 478 | EJX50427.1 | 14685 | 20042 | 161 | EJX50429.1 | 14686 | 20043 | 283 | | | | |
| 657 | 20044 | 478 | EJX67855.1 | 14688 | 20045 | 161 | EJX67853.1 | 14689 | 20046 | 283 | | | | |
| 658 | 20047 | 478 | EJX96646.1 | 14691 | 20048 | 161 | EJX96648.1 | 14692 | 20049 | 283 | | | | |
| 659 | 20050 | 478 | EJX94482.1 | 14694 | 20051 | 161 | EJX94480.1 | 14695 | 20052 | 283 | | | | |
| 660 | 20053 | 478 | EJX98619.1 | 14697 | 20054 | 161 | EJX98617.1 | 14698 | 20055 | 283 | | | | |
| 661 | 20056 | 478 | EJY06339.1 | 14700 | 20057 | 161 | EJY06337.1 | 14701 | 20058 | 283 | | | | |
| 662 | 20059 | 478 | EJY12739.1 | 14703 | 20060 | 161 | EJY12737.1 | 14704 | 20061 | 283 | | | | |
| 663 | 20062 | 478 | EJY15299.1 | 14706 | 20063 | 161 | EJY15301.1 | 14707 | 20064 | 283 | | | | |
| 664 | 20065 | 214 | EKB22092.1 | 14709 | 20066 | 164 | EKB22094.1 | 14710 | 20067 | 181 | | | | |
| 665 | 20068 | 494 | EKG43672.1 | 14712 | 20069 | 325 | | | | | | | | |
| 666 | 20070 | 481 | EYD87720.1 | 14714 | 20071 | 155 | | | | | | | | |
| 667 | 20072 | 491 | EZR33641.1 | 14716 | 20073 | 202 | EZR33642.1 | 14717 | 20074 | 308 | | | | |
| 668 | 20075 | 483 | EZR32905.1 | 14719 | 20076 | 202 | EZR32906.1 | 14720 | 20077 | 72 | | | | |
| 669 | 20078 | 483 | EZR32077.1 | 14722 | 20079 | 202 | EZR32078.1 | 14723 | 20080 | 308 | | | | |
| 670 | 20081 | 483 | EZR40122.1 | 14725 | 20082 | 202 | EZR40123.1 | 14726 | 20083 | 308 | | | | |
| 671 | 20084 | 483 | EZR39625.1 | 14728 | 20085 | 202 | EZR39626.1 | 14729 | 20086 | 308 | | | | |
| 672 | 20087 | 483 | EZR44609.1 | 14731 | 20088 | 202 | EZR44610.1 | 14732 | 20089 | 308 | | | | |
| 673 | 20090 | 483 | EZR45905.1 | 14734 | 20091 | 202 | EZR45906.1 | 14735 | 20092 | 308 | | | | |
| 674 | 20093 | 483 | EZR45666.1 | 14737 | 20094 | 202 | EZR45667.1 | 14738 | 20095 | 308 | | | | |
| 675 | 20096 | 483 | EZR50023.1 | 14740 | 20097 | 202 | EZR50024.1 | 14741 | 20098 | 308 | | | | |
| 676 | 20099 | 483 | EZR55033.1 | 14743 | 20100 | 202 | EZR55034.1 | 14744 | 20101 | 308 | | | | |
| 677 | 20102 | 131 | EZR57943.1 | 14746 | 20103 | 202 | EZR57944.1 | 14747 | 20104 | 308 | | | | |
| 678 | 20105 | 483 | EZR61547.1 | 14749 | 20106 | 202 | EZR61548.1 | 14750 | 20107 | 308 | | | | |
| 679 | 20108 | 483 | EZR61334.1 | 14752 | 20109 | 202 | EZR61335.1 | 14753 | 20110 | 308 | | | | |
| 680 | 20111 | 483 | EZR64329.1 | 14755 | 20112 | 202 | EZR64330.1 | 14756 | 20113 | 308 | | | | |
| 681 | 20114 | 483 | EZR68239.1 | 14758 | 20115 | 202 | EZR68240.1 | 14759 | 20116 | 308 | | | | |
| 682 | 20117 | 483 | EZR69012.1 | 14761 | 20118 | 202 | EZR69013.1 | 14762 | 20119 | 308 | | | | |
| 683 | 20120 | 483 | EZR71839.1 | 14764 | 20121 | 202 | EZR71840.1 | 14765 | 20122 | 308 | | | | |
| 684 | 20123 | 483 | EZR76989.1 | 14767 | 20124 | 202 | EZR76990.1 | 14768 | 20125 | 308 | | | | |
| 685 | 20126 | 483 | EZR83386.1 | 14770 | 20127 | 202 | EZR83387.1 | 14771 | 20128 | 308 | | | | |
| 686 | 20129 | 483 | EZR85550.1 | 14773 | 20130 | 202 | EZR85551.1 | 14774 | 20131 | 308 | | | | |
| 687 | 20132 | 483 | EZR86480.1 | 14776 | 20133 | 202 | EZR86481.1 | 14777 | 20134 | 308 | | | | |
| 688 | 20135 | 483 | EZR91405.1 | 14779 | 20136 | 202 | EZR91406.1 | 14780 | 20137 | 308 | | | | |
| 689 | 20138 | 375 | EZR97098.1 | 14782 | 20139 | 202 | EZR97099.1 | 14783 | 20140 | 308 | | | | |
| 690 | 20141 | 483 | EZR91806.1 | 14785 | 20142 | 202 | EZR91807.1 | 14786 | 20143 | 308 | | | | |
| 691 | 20144 | 483 | EZS01236.1 | 14788 | 20145 | 202 | EZS01237.1 | 14789 | 20146 | 308 | | | | |
| 692 | 20147 | 483 | EZR98551.1 | 14791 | 20148 | 202 | EZR98552.1 | 14792 | 20149 | 308 | | | | |
| 693 | 20150 | 483 | EZS03611.1 | 14794 | 20151 | 202 | EZS03612.1 | 14795 | 20152 | 308 | | | | |
| 694 | 20153 | 483 | EZS03315.1 | 14797 | 20154 | 202 | EZS03316.1 | 14798 | 20155 | 308 | | | | |
| 695 | 20156 | 483 | EZS19478.1 | 14800 | 20157 | 202 | EZS19479.1 | 14801 | 20158 | 308 | | | | |
| 696 | 20159 | 483 | EZS08902.1 | 14803 | 20160 | 202 | EZS08903.1 | 14804 | 20161 | 308 | | | | |
| 697 | 20162 | 483 | EZS11742.1 | 14806 | 20163 | 202 | EZS11743.1 | 14807 | 20164 | 308 | | | | |
| 698 | 20165 | 483 | EZS15733.1 | 14809 | 20166 | 202 | EZS15734.1 | 14810 | 20167 | 308 | | | | |
| 699 | 20168 | 483 | EZS17714.1 | 14812 | 20169 | 202 | EZS17715.1 | 14813 | 20170 | 308 | | | | |
| 700 | 20171 | 483 | EZS22503.1 | 14815 | 20172 | 202 | EZS22504.1 | 14816 | 20173 | 308 | | | | |
| 701 | 20174 | 483 | EZS28194.1 | 14818 | 20175 | 202 | EZS28195.1 | 14819 | 20176 | 308 | | | | |
| 702 | 20177 | 483 | EZS23870.1 | 14821 | 20178 | 202 | EZS23871.1 | 14822 | 20179 | 308 | | | | |
| 703 | 20180 | 483 | EZS29420.1 | 14824 | 20181 | 202 | EZS29421.1 | 14825 | 20182 | 308 | | | | |
| 704 | 20183 | 483 | EZS31412.1 | 14827 | 20184 | 202 | EZS31413.1 | 14828 | 20185 | 308 | | | | |
| 705 | 20186 | 483 | EZS33211.1 | 14830 | 20187 | 202 | EZS33212.1 | 14831 | 20188 | 308 | | | | |
| 706 | 20189 | 483 | EZS35183.1 | 14833 | 20190 | 202 | EZS35184.1 | 14834 | 20191 | 308 | | | | |
| 707 | 20192 | 483 | EZS39160.1 | 14836 | 20193 | 202 | EZS39161.1 | 14837 | 20194 | 308 | | | | |
| 708 | 20195 | 483 | EZS43275.1 | 14839 | 20196 | 202 | EZS43276.1 | 14840 | 20197 | 308 | | | | |
| 709 | 20198 | 483 | EZS45536.1 | 14842 | 20199 | 202 | EZS45537.1 | 14843 | 20200 | 308 | | | | |
| 710 | 20201 | 483 | EZS50845.1 | 14845 | 20202 | 202 | EZS50846.1 | 14846 | 20203 | 308 | | | | |
| 711 | 20204 | 483 | EZS49645.1 | 14848 | 20205 | 202 | EZS49646.1 | 14849 | 20206 | 308 | | | | |
| 712 | 20207 | 483 | EZS53446.1 | 14851 | 20208 | 202 | EZS53447.1 | 14852 | 20209 | 308 | | | | |
| 713 | 20210 | 483 | EZS57830.1 | 14854 | 20211 | 202 | EZS57831.1 | 14855 | 20212 | 308 | | | | |
| 714 | 20213 | 483 | EZS59103.1 | 14857 | 20214 | 202 | EZS59104.1 | 14858 | 20215 | 308 | | | | |
| 715 | 20216 | 483 | EZS59926.1 | 14860 | 20217 | 202 | EZS59927.1 | 14861 | 20218 | 308 | | | | |
| 716 | 20219 | 483 | EZS62656.1 | 14863 | 20220 | 202 | EZS62657.1 | 14864 | 20221 | 308 | | | | |
| 717 | 20222 | 483 | EZS64790.1 | 14866 | 20223 | 202 | EZS64791.1 | 14867 | 20224 | 308 | | | | |
| 718 | 20225 | 483 | EZS69841.1 | 14869 | 20226 | 202 | EZS69842.1 | 14870 | 20227 | 308 | | | | |
| 719 | 20228 | 483 | EZS69136.1 | 14872 | 20229 | 202 | EZS69137.1 | 14873 | 20230 | 308 | | | | |
| 720 | 20231 | 483 | EZS72360.1 | 14875 | 20232 | 202 | EZS72361.1 | 14876 | 20233 | 308 | | | | |
| 721 | 20234 | 483 | EZS75505.1 | 14878 | 20235 | 202 | EZS75506.1 | 14879 | 20236 | 308 | | | | |
| 722 | 20237 | 483 | EZS76926.1 | 14881 | 20238 | 202 | EZS76927.1 | 14882 | 20239 | 308 | | | | |
| 723 | 20240 | 483 | EZS80905.1 | 14884 | 20241 | 202 | EZS80906.1 | 14885 | 20242 | 308 | | | | |
| 724 | 20243 | 483 | EZS87843.1 | 14887 | 20244 | 202 | EZS87844.1 | 14888 | 20245 | 308 | | | | |
| 725 | 20246 | 483 | EZS84436.1 | 14890 | 20247 | 202 | EZS84437.1 | 14891 | 20248 | 308 | | | | |
| 726 | 20249 | 483 | EZS87767.1 | 14893 | 20250 | 202 | EZS87768.1 | 14894 | 20251 | 308 | | | | |
| 727 | 20252 | 483 | EZS90161.1 | 14896 | 20253 | 202 | EZS90162.1 | 14897 | 20254 | 308 | | | | |
| 728 | 20255 | 483 | EZS94700.1 | 14899 | 20256 | 202 | EZS94701.1 | 14900 | 20257 | 308 | | | | |
| 729 | 20258 | 483 | EZS98279.1 | 14902 | 20259 | 202 | EZS98280.1 | 14903 | 20260 | 308 | | | | |
| 730 | 20261 | 483 | EZT00069.1 | 14905 | 20262 | 202 | EZT00070.1 | 14906 | 20263 | 308 | | | | |
| 731 | 20264 | 483 | EZT04299.1 | 14908 | 20265 | 202 | EZT04300.1 | 14909 | 20266 | 308 | | | | |
| 732 | 20267 | 483 | EZT04245.1 | 14911 | 20268 | 202 | EZT04246.1 | 14912 | 20269 | 308 | | | | |
| 733 | 20270 | 483 | EZT06688.1 | 14914 | 20271 | 202 | EZT06689.1 | 14915 | 20272 | 308 | | | | |
| 734 | 20273 | 483 | EZT09550.1 | 14917 | 20274 | 202 | EZT09551.1 | 14918 | 20275 | 308 | | | | |
| 735 | 20276 | 483 | EZT09959.1 | 14920 | 20277 | 202 | EZT09960.1 | 14921 | 20278 | 308 | | | | |
| 736 | 20279 | 483 | EZT16278.1 | 14923 | 20280 | 202 | EZT16279.1 | 14924 | 20281 | 308 | | | | |
| 737 | 20282 | 483 | EZT18986.1 | 14926 | 20283 | 202 | EZT18987.1 | 14927 | 20284 | 308 | | | | |
| 738 | 20285 | 483 | EZT18345.1 | 14929 | 20286 | 202 | EZT18346.1 | 14930 | 20287 | 308 | | | | |
| 739 | 20288 | 483 | EZT23521.1 | 14932 | 20289 | 202 | EZT23522.1 | 14933 | 20290 | 308 | | | | |
| 740 | 20291 | 483 | EZT25330.1 | 14935 | 20292 | 202 | EZT25331.1 | 14936 | 20293 | 308 | | | | |
| 741 | 20294 | 375 | EZT27372.1 | 14938 | 20295 | 202 | EZT27373.1 | 14939 | 20296 | 308 | | | | |
| 742 | 20297 | 483 | EZT32066.1 | 14941 | 20298 | 202 | EZT32067.1 | 14942 | 20299 | 308 | | | | |
| 743 | 20300 | 483 | EZT32805.1 | 14944 | 20301 | 202 | EZT32806.1 | 14945 | 20302 | 308 | | | | |
| 744 | 20303 | 483 | EZT35385.1 | 14947 | 20304 | 202 | EZT35386.1 | 14948 | 20305 | 308 | | | | |
| 745 | 20306 | 483 | EZT42044.1 | 14950 | 20307 | 202 | EZT42045.1 | 14951 | 20308 | 308 | | | | |
| 746 | 20309 | 483 | EZT39170.1 | 14953 | 20310 | 202 | EZT39171.1 | 14954 | 20311 | 308 | | | | |
| 747 | 20312 | 483 | EZT44506.1 | 14956 | 20313 | 202 | EZT44507.1 | 14957 | 20314 | 308 | | | | |
| 748 | 20315 | 375 | EZT47448.1 | 14959 | 20316 | 202 | EZT47449.1 | 14960 | 20317 | 308 | | | | |
| 749 | 20318 | 375 | EZT50533.1 | 14962 | 20319 | 202 | EZT50534.1 | 14963 | 20320 | 308 | | | | |
| 750 | 20321 | 483 | EZT52623.1 | 14965 | 20322 | 202 | EZT52624.1 | 14966 | 20323 | 308 | | | | |
| 751 | 20324 | 483 | EZT54278.1 | 14968 | 20325 | 202 | EZT54279.1 | 14969 | 20326 | 308 | | | | |
| 752 | 20327 | 483 | EZT57809.1 | 14971 | 20328 | 202 | EZT57810.1 | 14972 | 20329 | 308 | | | | |
| 753 | 20330 | 483 | EZT60955.1 | 14974 | 20331 | 202 | EZT60956.1 | 14975 | 20332 | 308 | | | | |
| 754 | 20333 | 483 | EZT61424.1 | 14977 | 20334 | 202 | EZT61425.1 | 14978 | 20335 | 308 | | | | |
| 755 | 20336 | 483 | EZT65755.1 | 14980 | 20337 | 202 | EZT65756.1 | 14981 | 20338 | 308 | | | | |
| 756 | 20339 | 483 | EZT68343.1 | 14983 | 20340 | 202 | EZT68344.1 | 14984 | 20341 | 308 | | | | |
| 757 | 20342 | 483 | EZT68416.1 | 14986 | 20343 | 202 | EZT68417.1 | 14987 | 20344 | 308 | | | | |
| 758 | 20345 | 483 | EZT73753.1 | 14989 | 20346 | 202 | EZT73754.1 | 14990 | 20347 | 308 | | | | |
| 759 | 20348 | 483 | EZT75320.1 | 14992 | 20349 | 202 | EZT75321.1 | 14993 | 20350 | 308 | | | | |
| 760 | 20351 | 483 | EZT77628.1 | 14995 | 20352 | 202 | EZT77629.1 | 14996 | 20353 | 308 | | | | |
| 761 | 20354 | 483 | EZT80085.1 | 14998 | 20355 | 202 | EZT80086.1 | 14999 | 20356 | 308 | | | | |
| 762 | 20357 | 483 | EZT82071.1 | 15001 | 20358 | 202 | EZT82072.1 | 15002 | 20359 | 308 | | | | |
| 763 | 20360 | 483 | EZT89288.1 | 15004 | 20361 | 202 | EZT89289.1 | 15005 | 20362 | 308 | | | | |
| 764 | 20363 | 483 | EZT93144.1 | 15007 | 20364 | 202 | EZT93145.1 | 15008 | 20365 | 308 | | | | |
| 765 | 20366 | 483 | EZT96079.1 | 15010 | 20367 | 202 | EZT96080.1 | 15011 | 20368 | 308 | | | | |
| 766 | 20369 | 483 | EZT99580.1 | 15013 | 20370 | 202 | EZT99581.1 | 15014 | 20371 | 308 | | | | |
| 767 | 20372 | 483 | EZU00839.1 | 15016 | 20373 | 202 | EZU00840.1 | 15017 | 20374 | 308 | | | | |
| 768 | 20375 | 483 | EZU03292.1 | 15019 | 20376 | 202 | EZU03293.1 | 15020 | 20377 | 308 | | | | |
| 769 | 20378 | 483 | EZU07138.1 | 15022 | 20379 | 202 | EZU07139.1 | 15023 | 20380 | 308 | | | | |
| 770 | 20381 | 483 | EZU08870.1 | 15025 | 20382 | 202 | EZU08871.1 | 15026 | 20383 | 308 | | | | |
| 771 | 20384 | 481 | EZK05851.1 | 15028 | 20385 | 155 | | | | | | | | |
| 772 | 20386 | 481 | EZK20523.1 | 15030 | 20387 | 155 | | | | | | | | |
| 773 | 20388 | 483 | KEA54803.1 | 15032 | 20389 | 165 | KEA54805.1 | 15033 | 20390 | 184 | | | | |
| 774 | 20391 | 132 | KGK10825.1 | 15035 | 20392 | 166 | | | | | | | | |
| 775 | 20393 | 479 | KDN56467.1 | 15037 | 20394 | 203 | KDN56465.1 | 15038 | 20395 | 182 | | | | |
| 776 | 20396 | 471 | KDS43696.1 | 15040 | 20397 | 203 | KDS43698.1 | 15041 | 20398 | 182 | | | | |
| 777 | 20399 | 481 | KDU61956.1 | 15043 | 20400 | 155 | | | | | | | | |
| 778 | 20401 | 529 | KDX77548.1 | 15045 | 20402 | 323 | | | | | | | | |
| 779 | 20403 | 529 | KDX82580.1 | 15047 | 20404 | 323 | | | | | | | | |
| 780 | 20405 | 481 | KDY27429.1 | 15049 | 20406 | 155 | | | | | | | | |
| 781 | 20407 | 481 | KED12525.1 | 15051 | 20408 | 191 | | | | | | | | |
| 782 | 20409 | 481 | KED15840.1 | 15053 | 20410 | 191 | | | | | | | | |
| 783 | 20411 | 481 | KED29322.1 | 15055 | 20412 | 200 | KED29321.1 | 15056 | 20413 | 191 | | | | |
| 784 | 20414 | 481 | KED25552.1 | 15058 | 20415 | 200 | KED25551.1 | 15059 | 20416 | 191 | | | | |
| 785 | 20417 | 481 | KED36895.1 | 15061 | 20418 | 200 | KED36894.1 | 15062 | 20419 | 191 | | | | |
| 786 | 20420 | 481 | KED34399.1 | 15064 | 20421 | 200 | | | | | | | | |
| 787 | 20422 | 475 | KED69848.1 | 15066 | 20423 | 228 | | | | | | | | |
| 788 | 20424 | 474 | KGK23634.1 | 15068 | 20425 | 163 | KGK23633.1 | 15069 | 20426 | 171 | | | | |
| 789 | 20427 | 481 | KEK91461.1 | 15071 | 20428 | 155 | | | | | | | | |
| 790 | 20429 | 481 | KEK94905.1 | 15073 | 20430 | 155 | | | | | | | | |
| 791 | 20431 | 299 | KEK94965.1 | 15075 | 20432 | 155 | | | | | | | | |
| 792 | 20433 | 311 | KEK89445.1 | 15077 | 20434 | 155 | | | | | | | | |
| 793 | 20435 | 294 | KEQ37437.1 | 15079 | 20436 | 206 | KEQ37440.1 | 15080 | 20437 | 180 | | | | |
| 794 | 20438 | 469 | KEQ45049.1 | 15082 | 20439 | 206 | KEQ45047.1 | 15083 | 20440 | 310 | | | | |
| 795 | 20441 | 484 | KFB55437.1 | 15085 | 20442 | 163 | | | | | | | | |
| 796 | 20443 | 484 | KFF79467.1 | 15087 | 20444 | 163 | | | | | | | | |
| 797 | 20445 | 475 | KFF62083.1 | 15089 | 20446 | 205 | KFF62085.1 | 15090 | 20447 | 200 | KFF62086.1 | 15091 | 20448 | 395 |
| 798 | 20450 | 486 | KTA63995.1 | 15094 | 20451 | 202 | | | | | | | | |
| 799 | 20452 | 466 | KNE78571.1 | 15096 | 20453 | 214 | | | | | | | | |
| 800 | 20454 | 477 | KFK51886.1 | 15098 | 20455 | 165 | KFK51888.1 | 15099 | 20456 | 225 | | | | |
| 801 | 20457 | 482 | KGA25875.1 | 15101 | 20458 | 200 | | | | | | | | |
| 802 | 20459 | 602 | KGN93840.1 | 15103 | 20460 | 391 | | | | | | | | |
| 803 | 20461 | 478 | KGK75436.1 | 15105 | 20462 | 161 | KGK75438.1 | 15106 | 20463 | 283 | | | | |
| 804 | 20464 | 471 | KGF38952.1 | 15108 | 20465 | 203 | KGF38954.1 | 15109 | 20466 | 182 | | | | |
| 805 | 20467 | 473 | KHD43838.1 | 15111 | 20468 | 166 | | | | | | | | |
| 806 | 20469 | 478 | KGQ80277.1 | 15113 | 20470 | 161 | KGQ80279.1 | 15114 | 20471 | 283 | | | | |
| 807 | 20472 | 211 | KIE90700.1 | 15116 | 20473 | 165 | KIE90687.1 | 15117 | 20474 | 214 | | | | |
| 808 | 20475 | 307 | KIE90615.1 | 15119 | 20476 | 165 | KIE90604.1 | 15120 | 20477 | 214 | | | | |
| 809 | 20478 | 307 | KIE97297.1 | 15122 | 20479 | 165 | KIE97287.1 | 15123 | 20480 | 214 | | | | |
| 810 | 20481 | 307 | KIE97677.1 | 15125 | 20482 | 165 | KIE97674.1 | 15126 | 20483 | 214 | | | | |
| 811 | 20484 | 307 | KIE96205.1 | 15128 | 20485 | 165 | KIE96201.1 | 15129 | 20486 | 214 | | | | |
| 812 | 20487 | 307 | KIE90369.1 | 15131 | 20488 | 165 | KIE90359.1 | 15132 | 20489 | 214 | | | | |
| 813 | 20490 | 487 | KHN56271.1 | 15134 | 20491 | 195 | KHN56274.1 | 15135 | 20492 | 165 | KHN56277.1 | 15136 | 20493 | 167 |
| 814 | 20495 | 483 | KII20610.1 | 15139 | 20496 | 202 | KII20611.1 | 15140 | 20497 | 308 | | | | |
| 815 | 20498 | 478 | KIE70776.1 | 15142 | 20499 | 203 | | | | | | | | |
| 816 | 20500 | 103 | KGY52719.1 | 15144 | 20501 | 192 | | | | | | | | |
| 817 | 20502 | 484 | KGZ13595.1 | 15146 | 20503 | 163 | | | | | | | | |
| 818 | 20504 | 480 | KHM45256.1 | 15148 | 20505 | 166 | | | | | | | | |
| 819 | 20506 | 480 | KID39106.1 | 15150 | 20507 | 166 | | | | | | | | |
| 820 | 20508 | 484 | KHO41400.1 | 15152 | 20509 | 163 | | | | | | | | |
| 821 | 20510 | 493 | KIM13726.1 | 15154 | 20511 | 156 | | | | | | | | |
| 822 | 20512 | 591 | KMM85343.1 | 15156 | 20513 | 322 | | | | | | | | |
| 823 | 20514 | 478 | ELA78318.1 | 15158 | 20515 | 161 | ELA78316.1 | 15159 | 20516 | 283 | | | | |
| 824 | 20517 | 478 | ELB72565.1 | 15161 | 20518 | 161 | ELB72563.1 | 15162 | 20519 | 283 | | | | |
| 825 | 20520 | 478 | ELB70912.1 | 15164 | 20521 | 161 | ELB70910.1 | 15165 | 20522 | 283 | | | | |
| 826 | 20523 | 478 | ELB76088.1 | 15167 | 20524 | 161 | ELB76086.1 | 15168 | 20525 | 283 | | | | |
| 827 | 20526 | 478 | ELB84572.1 | 15170 | 20527 | 161 | ELB84570.1 | 15171 | 20528 | 283 | | | | |
| 828 | 20529 | 478 | ELB85484.1 | 15173 | 20530 | 161 | ELB85482.1 | 15174 | 20531 | 283 | | | | |
| 829 | 20532 | 596 | EKS34036.1 | 15176 | 20533 | 370 | | | | | | | | |
| 830 | 20534 | 478 | ELC28355.1 | 15178 | 20535 | 203 | | | | | | | | |
| 831 | 20536 | 478 | ELD53294.1 | 15180 | 20537 | 203 | | | | | | | | |
| 832 | 20538 | 478 | ELF38885.1 | 15182 | 20539 | 203 | | | | | | | | |
| 833 | 20540 | 126 | ELF73879.1 | 15184 | 20541 | 155 | | | | | | | | |
| 834 | 20542 | 478 | ELH15112.1 | 15186 | 20543 | 203 | | | | | | | | |
| 835 | 20544 | 478 | ELF82584.1 | 15188 | 20545 | 203 | | | | | | | | |
| 836 | 20546 | 329 | ENU62566.1 | 15190 | 20547 | 160 | ENU62743.1 | 15191 | 20548 | 179 | | | | |
| 837 | 20549 | 476 | ENX56756.1 | 15193 | 20550 | 145 | ENX56757.1 | 15194 | 20551 | 269 | | | | |
| 838 | 20552 | 515 | ENX18769.1 | 15196 | 20553 | 778 | | | | | | | | |
| 839 | 20554 | 257 | ENX27878.1 | 15198 | 20555 | 160 | ENX27882.1 | 15199 | 20556 | 179 | | | | |
| 840 | 20557 | 495 | ENZ03438.1 | 15201 | 20558 | 160 | ENZ03439.1 | 15202 | 20559 | 193 | | | | |
| 841 | 20560 | 481 | ENZ02554.1 | 15204 | 20561 | 227 | ENZ02556.1 | 15205 | 20562 | 394 | | | | |
| 842 | 20563 | 478 | EOH58475.1 | 15207 | 20564 | 161 | EOH58477.1 | 15208 | 20565 | 283 | | | | |
| 843 | 20566 | 478 | EOL74787.1 | 15210 | 20567 | 161 | EOL74785.1 | 15211 | 20568 | 283 | | | | |
| 844 | 20569 | 478 | EOI52381.1 | 15213 | 20570 | 161 | EOI52383.1 | 15214 | 20571 | 283 | | | | |
| 845 | 20572 | 478 | EOG00797.1 | 15216 | 20573 | 161 | EOG00795.1 | 15217 | 20574 | 283 | | | | |
| 846 | 20575 | 478 | EOG01000.1 | 15219 | 20576 | 161 | EOG00998.1 | 15220 | 20577 | 283 | | | | |
| 847 | 20578 | 478 | EOG17545.1 | 15222 | 20579 | 161 | EOG17543.1 | 15223 | 20580 | 283 | | | | |
| 848 | 20581 | 478 | EOG17726.1 | 15225 | 20582 | 161 | EOG17728.1 | 15226 | 20583 | 283 | | | | |
| 849 | 20584 | 478 | EOG29142.1 | 15228 | 20585 | 161 | EOG29140.1 | 15229 | 20586 | 283 | | | | |
| 850 | 20587 | 478 | EOM31820.1 | 15231 | 20588 | 161 | EOM31822.1 | 15232 | 20589 | 283 | | | | |
| 851 | 20590 | 491 | EOQ01915.1 | 15234 | 20591 | 202 | | | | | | | | |
| 852 | 20592 | 473 | EOT43558.1 | 15236 | 20593 | 165 | | | | | | | | |
| 853 | 20594 | 473 | EOW87388.1 | 15238 | 20595 | 165 | | | | | | | | |
| 854 | 20596 | 482 | EPD26425.1 | 15240 | 20597 | 217 | EPD26427.1 | 15241 | 20598 | 167 | EPD26426.1 | 15242 | 20599 | 187 |
| 855 | 20600 | 482 | EPI22404.1 | 15244 | 20601 | 217 | | | | | | | | |
| 856 | 20602 | 478 | EQN31626.1 | 15246 | 20603 | 203 | | | | | | | | |
| 857 | 20604 | 478 | EQO47877.1 | 15248 | 20605 | 203 | | | | | | | | |
| 858 | 20606 | 482 | EQO88545.1 | 15250 | 20607 | 166 | | | | | | | | |
| 859 | 20608 | 482 | EQS52096.1 | 15252 | 20609 | 166 | | | | | | | | |
| 860 | 20610 | 482 | EQX21242.1 | 15254 | 20611 | 166 | | | | | | | | |
| 861 | 20612 | 488 | ERK54361.1 | 15256 | 20613 | 167 | | | | | | | | |
| 862 | 20614 | 487 | ERW54757.1 | 15258 | 20615 | 167 | ERW54755.1 | 15259 | 20616 | 304 | | | | |
| 863 | 20617 | 487 | ERW94008.1 | 15261 | 20618 | 167 | ERW94010.1 | 15262 | 20619 | 304 | | | | |
| 864 | 20620 | 469 | ETD07629.1 | 15264 | 20621 | 206 | ETD07627.1 | 15265 | 20622 | 388 | | | | |
| 865 | 20623 | 478 | ETU12931.1 | 15267 | 20624 | 161 | ETU12933.1 | 15268 | 20625 | 283 | | | | |
| 866 | 20626 | 484 | ETX46916.1 | 15270 | 20627 | 163 | | | | | | | | |
| 867 | 20628 | 475 | EXX84013.1 | 15272 | 20629 | 228 | | | | | | | | |
| 868 | 20630 | 484 | EZQ69340.1 | 15274 | 20631 | 163 | | | | | | | | |
| 869 | 20632 | 483 | EZU15806.1 | 15276 | 20633 | 202 | EZU15807.1 | 15277 | 20634 | 308 | | | | |
| 870 | 20635 | 483 | EZU14923.1 | 15279 | 20636 | 202 | EZU14924.1 | 15280 | 20637 | 308 | | | | |
| 871 | 20638 | 483 | EZU22367.1 | 15282 | 20639 | 202 | EZU22368.1 | 15283 | 20640 | 308 | | | | |
| 872 | 20641 | 483 | EZU24875.1 | 15285 | 20642 | 202 | EZU24876.1 | 15286 | 20643 | 308 | | | | |
| 873 | 20644 | 483 | EZU30072.1 | 15288 | 20645 | 202 | EZU30073.1 | 15289 | 20646 | 308 | | | | |
| 874 | 20647 | 483 | EZU33074.1 | 15291 | 20648 | 202 | EZU33075.1 | 15292 | 20649 | 308 | | | | |
| 875 | 20650 | 483 | EZU39421.1 | 15294 | 20651 | 202 | EZU39422.1 | 15295 | 20652 | 308 | | | | |
| 876 | 20653 | 483 | EZU35475.1 | 15297 | 20654 | 202 | EZU35476.1 | 15298 | 20655 | 308 | | | | |
| 877 | 20656 | 483 | EZU41789.1 | 15300 | 20657 | 202 | EZU41790.1 | 15301 | 20658 | 308 | | | | |
| 878 | 20659 | 483 | EZU42122.1 | 15303 | 20660 | 202 | EZU42123.1 | 15304 | 20661 | 308 | | | | |
| 879 | 20662 | 483 | EZU46567.1 | 15306 | 20663 | 202 | EZU46568.1 | 15307 | 20664 | 308 | | | | |
| 880 | 20665 | 483 | EZU46768.1 | 15309 | 20666 | 202 | EZU46769.1 | 15310 | 20667 | 308 | | | | |
| 881 | 20668 | 483 | EZU48613.1 | 15312 | 20669 | 202 | EZU48614.1 | 15313 | 20670 | 308 | | | | |
| 882 | 20671 | 483 | EZU52016.1 | 15315 | 20672 | 202 | EZU52017.1 | 15316 | 20673 | 308 | | | | |
| 883 | 20674 | 483 | EZU55225.1 | 15318 | 20675 | 202 | EZU55226.1 | 15319 | 20676 | 308 | | | | |
| 884 | 20677 | 483 | EZU56166.1 | 15321 | 20678 | 202 | EZU56167.1 | 15322 | 20679 | 308 | | | | |
| 885 | 20680 | 483 | EZU61335.1 | 15324 | 20681 | 202 | EZU61336.1 | 15325 | 20682 | 308 | | | | |
| 886 | 20683 | 483 | EZU65298.1 | 15327 | 20684 | 202 | EZU65299.1 | 15328 | 20685 | 308 | | | | |
| 887 | 20686 | 483 | EZU73660.1 | 15330 | 20687 | 202 | EZU73661.1 | 15331 | 20688 | 308 | | | | |
| 888 | 20689 | 483 | EZU80185.1 | 15333 | 20690 | 202 | EZU80186.1 | 15334 | 20691 | 308 | | | | |
| 889 | 20692 | 483 | EZU80947.1 | 15336 | 20693 | 202 | EZU80948.1 | 15337 | 20694 | 308 | | | | |
| 890 | 20695 | 483 | EZU82979.1 | 15339 | 20696 | 202 | EZU82980.1 | 15340 | 20697 | 308 | | | | |
| 891 | 20698 | 483 | EZU87060.1 | 15342 | 20699 | 202 | EZU87061.1 | 15343 | 20700 | 308 | | | | |
| 892 | 20701 | 483 | EZU88263.1 | 15345 | 20702 | 202 | EZU88264.1 | 15346 | 20703 | 308 | | | | |
| 893 | 20704 | 483 | EZU90292.1 | 15348 | 20705 | 202 | EZU90293.1 | 15349 | 20706 | 308 | | | | |
| 894 | 20707 | 483 | EZU94766.1 | 15351 | 20708 | 202 | EZU94767.1 | 15352 | 20709 | 308 | | | | |
| 895 | 20710 | 483 | EZU96099.1 | 15354 | 20711 | 202 | EZU96100.1 | 15355 | 20712 | 308 | | | | |
| 896 | 20713 | 483 | EZV02406.1 | 15357 | 20714 | 202 | EZV02407.1 | 15358 | 20715 | 308 | | | | |
| 897 | 20716 | 375 | EZV09966.1 | 15360 | 20717 | 202 | EZV09967.1 | 15361 | 20718 | 308 | | | | |
| 898 | 20719 | 483 | EZV11273.1 | 15363 | 20720 | 202 | EZV11274.1 | 15364 | 20721 | 308 | | | | |
| 899 | 20722 | 483 | EZV17196.1 | 15366 | 20723 | 202 | EZV17197.1 | 15367 | 20724 | 308 | | | | |
| 900 | 20725 | 483 | EZV18373.1 | 15369 | 20726 | 202 | EZV18374.1 | 15370 | 20727 | 308 | | | | |
| 901 | 20728 | 483 | EZV22689.1 | 15372 | 20729 | 202 | EZV22690.1 | 15373 | 20730 | 308 | | | | |
| 902 | 20731 | 483 | EZV23633.1 | 15375 | 20732 | 202 | EZV23634.1 | 15376 | 20733 | 308 | | | | |
| 903 | 20734 | 483 | EZV30019.1 | 15378 | 20735 | 202 | EZV30020.1 | 15379 | 20736 | 308 | | | | |
| 904 | 20737 | 483 | EZV34238.1 | 15381 | 20738 | 202 | EZV34239.1 | 15382 | 20739 | 308 | | | | |
| 905 | 20740 | 483 | EZV37877.1 | 15384 | 20741 | 202 | EZV37878.1 | 15385 | 20742 | 308 | | | | |
| 906 | 20743 | 483 | EZV41497.1 | 15387 | 20744 | 202 | EZV41498.1 | 15388 | 20745 | 308 | | | | |
| 907 | 20746 | 483 | EZV40171.1 | 15390 | 20747 | 202 | EZV40172.1 | 15391 | 20748 | 308 | | | | |
| 908 | 20749 | 483 | EZV45689.1 | 15393 | 20750 | 202 | EZV45690.1 | 15394 | 20751 | 308 | | | | |
| 909 | 20752 | 483 | EZV49481.1 | 15396 | 20753 | 202 | EZV49482.1 | 15397 | 20754 | 308 | | | | |
| 910 | 20755 | 483 | EZV51741.1 | 15399 | 20756 | 202 | EZV51742.1 | 15400 | 20757 | 308 | | | | |
| 911 | 20758 | 483 | EZV56159.1 | 15402 | 20759 | 202 | EZV56160.1 | 15403 | 20760 | 308 | | | | |
| 912 | 20761 | 483 | EZV60059.1 | 15405 | 20762 | 202 | EZV60060.1 | 15406 | 20763 | 308 | | | | |
| 913 | 20764 | 483 | EZV56177.1 | 15408 | 20765 | 202 | EZV56178.1 | 15409 | 20766 | 308 | | | | |
| 914 | 20767 | 483 | EZV62993.1 | 15411 | 20768 | 202 | EZV62994.1 | 15412 | 20769 | 308 | | | | |
| 915 | 20770 | 375 | EZV68411.1 | 15414 | 20771 | 202 | EZV68412.1 | 15415 | 20772 | 308 | | | | |
| 916 | 20773 | 483 | EZV73437.1 | 15417 | 20774 | 202 | EZV73438.1 | 15418 | 20775 | 308 | | | | |
| 917 | 20776 | 483 | EZV79300.1 | 15420 | 20777 | 202 | EZV79301.1 | 15421 | 20778 | 308 | | | | |
| 918 | 20779 | 483 | EZV78172.1 | 15423 | 20780 | 202 | EZV78173.1 | 15424 | 20781 | 308 | | | | |
| 919 | 20782 | 483 | EZV80244.1 | 15426 | 20783 | 202 | EZV80245.1 | 15427 | 20784 | 308 | | | | |
| 920 | 20785 | 483 | EZV86031.1 | 15429 | 20786 | 202 | EZV86032.1 | 15430 | 20787 | 308 | | | | |
| 921 | 20788 | 483 | EZV85083.1 | 15432 | 20789 | 202 | EZV85084.1 | 15433 | 20790 | 308 | | | | |
| 922 | 20791 | 483 | EZV88943.1 | 15435 | 20792 | 202 | EZV88944.1 | 15436 | 20793 | 308 | | | | |
| 923 | 20794 | 483 | EZV92665.1 | 15438 | 20795 | 202 | EZV92666.1 | 15439 | 20796 | 308 | | | | |
| 924 | 20797 | 483 | EZV94446.1 | 15441 | 20798 | 202 | EZV94447.1 | 15442 | 20799 | 308 | | | | |
| 925 | 20800 | 483 | EZV97124.1 | 15444 | 20801 | 202 | EZV97125.1 | 15445 | 20802 | 308 | | | | |
| 926 | 20803 | 483 | EZV98955.1 | 15447 | 20804 | 202 | EZV98956.1 | 15448 | 20805 | 308 | | | | |
| 927 | 20806 | 483 | EZW01626.1 | 15450 | 20807 | 202 | EZW01627.1 | 15451 | 20808 | 308 | | | | |
| 928 | 20809 | 483 | EZW04544.1 | 15453 | 20810 | 202 | EZW04545.1 | 15454 | 20811 | 308 | | | | |
| 929 | 20812 | 483 | EZW06683.1 | 15456 | 20813 | 202 | EZW06684.1 | 15457 | 20814 | 308 | | | | |
| 930 | 20815 | 483 | EZW08826.1 | 15459 | 20816 | 202 | EZW08827.1 | 15460 | 20817 | 308 | | | | |
| 931 | 20818 | 483 | EZW10958.1 | 15462 | 20819 | 202 | EZW10959.1 | 15463 | 20820 | 308 | | | | |
| 932 | 20821 | 483 | EZW14697.1 | 15465 | 20822 | 202 | EZW14698.1 | 15466 | 20823 | 308 | | | | |
| 933 | 20824 | 483 | EZW17374.1 | 15468 | 20825 | 202 | EZW17375.1 | 15469 | 20826 | 308 | | | | |
| 934 | 20827 | 483 | EZW21155.1 | 15471 | 20828 | 202 | EZW21156.1 | 15472 | 20829 | 308 | | | | |
| 935 | 20830 | 483 | EZW24845.1 | 15474 | 20831 | 202 | EZW24846.1 | 15475 | 20832 | 308 | | | | |
| 936 | 20833 | 483 | EZW30607.1 | 15477 | 20834 | 202 | EZW30608.1 | 15478 | 20835 | 308 | | | | |
| 937 | 20836 | 483 | EZW29482.1 | 15480 | 20837 | 202 | EZW29483.1 | 15481 | 20838 | 308 | | | | |
| 938 | 20839 | 483 | EZW34091.1 | 15483 | 20840 | 202 | EZW34092.1 | 15484 | 20841 | 308 | | | | |
| 939 | 20842 | 483 | EZW34358.1 | 15486 | 20843 | 202 | EZW34359.1 | 15487 | 20844 | 308 | | | | |
| 940 | 20845 | 483 | EZW38690.1 | 15489 | 20846 | 202 | EZW38691.1 | 15490 | 20847 | 308 | | | | |
| 941 | 20848 | 483 | EZW42267.1 | 15492 | 20849 | 202 | EZW42268.1 | 15493 | 20850 | 308 | | | | |
| 942 | 20851 | 483 | EZW41571.1 | 15495 | 20852 | 202 | EZW41572.1 | 15496 | 20853 | 308 | | | | |
| 943 | 20854 | 483 | EZW45922.1 | 15498 | 20855 | 202 | EZW45923.1 | 15499 | 20856 | 308 | | | | |
| 944 | 20857 | 483 | EZW48564.1 | 15501 | 20858 | 202 | EZW48565.1 | 15502 | 20859 | 308 | | | | |
| 945 | 20860 | 483 | EZW51954.1 | 15504 | 20861 | 202 | EZW51955.1 | 15505 | 20862 | 308 | | | | |
| 946 | 20863 | 483 | EZW52895.1 | 15507 | 20864 | 202 | EZW52896.1 | 15508 | 20865 | 308 | | | | |
| 947 | 20866 | 483 | EZW54602.1 | 15510 | 20867 | 202 | EZW54603.1 | 15511 | 20868 | 308 | | | | |
| 948 | 20869 | 483 | EZW61226.1 | 15513 | 20870 | 202 | EZW61227.1 | 15514 | 20871 | 308 | | | | |
| 949 | 20872 | 483 | EZW59347.1 | 15516 | 20873 | 202 | EZW59348.1 | 15517 | 20874 | 308 | | | | |
| 950 | 20875 | 483 | EZW64573.1 | 15519 | 20876 | 202 | EZW64574.1 | 15520 | 20877 | 308 | | | | |
| 951 | 20878 | 493 | EZW67817.1 | 15522 | 20879 | 202 | EZW67818.1 | 15523 | 20880 | 308 | | | | |
| 952 | 20881 | 483 | EZW72064.1 | 15525 | 20882 | 202 | EZW72065.1 | 15526 | 20883 | 308 | | | | |
| 953 | 20884 | 483 | EZW73743.1 | 15528 | 20885 | 202 | EZW73744.1 | 15529 | 20886 | 308 | | | | |
| 954 | 20887 | 483 | EZW78365.1 | 15531 | 20888 | 202 | EZW78366.1 | 15532 | 20889 | 308 | | | | |
| 955 | 20890 | 483 | EZW78066.1 | 15534 | 20891 | 202 | EZW78067.1 | 15535 | 20892 | 308 | | | | |
| 956 | 20893 | 483 | EZW85423.1 | 15537 | 20894 | 202 | EZW85424.1 | 15538 | 20895 | 308 | | | | |
| 957 | 20896 | 483 | EZW86267.1 | 15540 | 20897 | 202 | EZW86268.1 | 15541 | 20898 | 308 | | | | |
| 958 | 20899 | 483 | EZW90699.1 | 15543 | 20900 | 202 | EZW90700.1 | 15544 | 20901 | 308 | | | | |
| 959 | 20902 | 483 | EZW93346.1 | 15546 | 20903 | 202 | EZW93347.1 | 15547 | 20904 | 308 | | | | |
| 960 | 20905 | 483 | EZW94210.1 | 15549 | 20906 | 202 | EZW94211.1 | 15550 | 20907 | 308 | | | | |
| 961 | 20908 | 483 | EZW97013.1 | 15552 | 20909 | 202 | EZW97014.1 | 15553 | 20910 | 308 | | | | |
| 962 | 20911 | 483 | EZX00537.1 | 15555 | 20912 | 202 | EZX00538.1 | 15556 | 20913 | 308 | | | | |
| 963 | 20914 | 483 | EZX02275.1 | 15558 | 20915 | 202 | EZX02276.1 | 15559 | 20916 | 308 | | | | |
| 964 | 20917 | 483 | EZX04398.1 | 15561 | 20918 | 202 | EZX04399.1 | 15562 | 20919 | 308 | | | | |
| 965 | 20920 | 483 | EZX08870.1 | 15564 | 20921 | 202 | EZX08871.1 | 15565 | 20922 | 308 | | | | |
| 966 | 20923 | 483 | EZX10606.1 | 15567 | 20924 | 202 | EZX10607.1 | 15568 | 20925 | 308 | | | | |
| 967 | 20926 | 483 | EZX16968.1 | 15570 | 20927 | 202 | EZX16969.1 | 15571 | 20928 | 308 | | | | |
| 968 | 20929 | 483 | EZX19508.1 | 15573 | 20930 | 202 | EZX19509.1 | 15574 | 20931 | 308 | | | | |
| 969 | 20932 | 483 | EZX23728.1 | 15576 | 20933 | 202 | EZX23729.1 | 15577 | 20934 | 308 | | | | |
| 970 | 20935 | 483 | EZX26909.1 | 15579 | 20936 | 202 | EZX26910.1 | 15580 | 20937 | 308 | | | | |
| 971 | 20938 | 483 | EZX30318.1 | 15582 | 20939 | 202 | EZX30319.1 | 15583 | 20940 | 308 | | | | |
| 972 | 20941 | 375 | EZX30778.1 | 15585 | 20942 | 202 | EZX30779.1 | 15586 | 20943 | 308 | | | | |
| 973 | 20944 | 375 | EZX33809.1 | 15588 | 20945 | 202 | EZX33810.1 | 15589 | 20946 | 308 | | | | |
| 974 | 20947 | 375 | EZX38405.1 | 15591 | 20948 | 202 | EZX38406.1 | 15592 | 20949 | 308 | | | | |
| 975 | 20950 | 483 | EZX39862.1 | 15594 | 20951 | 202 | EZX39863.1 | 15595 | 20952 | 308 | | | | |
| 976 | 20953 | 493 | EZX38522.1 | 15597 | 20954 | 202 | EZX38523.1 | 15598 | 20955 | 308 | | | | |
| 977 | 20956 | 493 | EZX45513.1 | 15600 | 20957 | 202 | EZX45514.1 | 15601 | 20958 | 308 | | | | |
| 978 | 20959 | 483 | EZX46728.1 | 15603 | 20960 | 202 | EZX46729.1 | 15604 | 20961 | 72 | | | | |
| 979 | 20962 | 483 | EZX48303.1 | 15606 | 20963 | 202 | EZX48304.1 | 15607 | 20964 | 308 | | | | |
| 980 | 20965 | 483 | EZX53233.1 | 15609 | 20966 | 202 | EZX53234.1 | 15610 | 20967 | 308 | | | | |
| 981 | 20968 | 375 | EZX53547.1 | 15612 | 20969 | 202 | EZX53548.1 | 15613 | 20970 | 308 | | | | |
| 982 | 20971 | 483 | EZX56514.1 | 15615 | 20972 | 202 | EZX56515.1 | 15616 | 20973 | 308 | | | | |
| 983 | 20974 | 483 | EZX60546.1 | 15618 | 20975 | 202 | EZX60547.1 | 15619 | 20976 | 308 | | | | |
| 984 | 20977 | 483 | EZX62510.1 | 15621 | 20978 | 202 | EZX62511.1 | 15622 | 20979 | 308 | | | | |
| 985 | 20980 | 375 | EZX64342.1 | 15624 | 20981 | 202 | EZX64343.1 | 15625 | 20982 | 308 | | | | |
| 986 | 20983 | 483 | EZX67970.1 | 15627 | 20984 | 202 | EZX67971.1 | 15628 | 20985 | 308 | | | | |
| 987 | 20986 | 483 | EZX70981.1 | 15630 | 20987 | 202 | EZX70982.1 | 15631 | 20988 | 308 | | | | |
| 988 | 20989 | 483 | EZX70897.1 | 15633 | 20990 | 202 | EZX70898.1 | 15634 | 20991 | 308 | | | | |
| 989 | 20992 | 483 | EZX78543.1 | 15636 | 20993 | 202 | EZX78544.1 | 15637 | 20994 | 308 | | | | |
| 990 | 20995 | 483 | EZX83215.1 | 15639 | 20996 | 202 | EZX83216.1 | 15640 | 20997 | 308 | | | | |
| 991 | 20998 | 483 | EZX78166.1 | 15642 | 20999 | 202 | EZX78167.1 | 15643 | 21000 | 308 | | | | |
| 992 | 21001 | 483 | EZX83510.1 | 15645 | 21002 | 202 | EZX83511.1 | 15646 | 21003 | 308 | | | | |
| 993 | 21004 | 483 | EZX85562.1 | 15648 | 21005 | 202 | EZX85563.1 | 15649 | 21006 | 308 | | | | |
| 994 | 21007 | 483 | EZX88437.1 | 15651 | 21008 | 202 | EZX88438.1 | 15652 | 21009 | 308 | | | | |
| 995 | 21010 | 483 | EZX90792.1 | 15654 | 21011 | 202 | EZX90793.1 | 15655 | 21012 | 308 | | | | |
| 996 | 21013 | 483 | EZX93305.1 | 15657 | 21014 | 202 | EZX93306.1 | 15658 | 21015 | 308 | | | | |
| 997 | 21016 | 483 | EZX97300.1 | 15660 | 21017 | 202 | EZX97301.1 | 15661 | 21018 | 308 | | | | |
| 998 | 21019 | 102 | EZX98399.1 | 15663 | 21020 | 202 | EZX98400.1 | 15664 | 21021 | 308 | | | | |
| 999 | 21022 | 483 | EZY04104.1 | 15666 | 21023 | 202 | EZY04105.1 | 15667 | 21024 | 308 | | | | |
| 1000 | 21025 | 483 | EZY04546.1 | 15669 | 21026 | 202 | EZY04547.1 | 15670 | 21027 | 308 | | | | |
| 1001 | 21028 | 483 | EZY06600.1 | 15672 | 21029 | 202 | EZY06601.1 | 15673 | 21030 | 308 | | | | |
| 1002 | 21031 | 483 | EZY11330.1 | 15675 | 21032 | 202 | EZY11331.1 | 15676 | 21033 | 308 | | | | |
| 1003 | 21034 | 483 | EZY13790.1 | 15678 | 21035 | 202 | EZY13791.1 | 15679 | 21036 | 308 | | | | |
| 1004 | 21037 | 483 | EZY14130.1 | 15681 | 21038 | 202 | EZY14131.1 | 15682 | 21039 | 295 | | | | |
| 1005 | 21040 | 483 | EZY14214.1 | 15684 | 21041 | 202 | EZY14215.1 | 15685 | 21042 | 308 | | | | |
| 1006 | 21043 | 483 | EZY21812.1 | 15687 | 21044 | 202 | EZY21813.1 | 15688 | 21045 | 308 | | | | |
| 1007 | 21046 | 483 | EZY21672.1 | 15690 | 21047 | 202 | EZY21673.1 | 15691 | 21048 | 308 | | | | |
| 1008 | 21049 | 131 | EZY24517.1 | 15693 | 21050 | 202 | EZY24518.1 | 15694 | 21051 | 308 | | | | |
| 1009 | 21052 | 483 | EZY26571.1 | 15696 | 21053 | 202 | EZY26572.1 | 15697 | 21054 | 308 | | | | |
| 1010 | 21055 | 483 | EZY29471.1 | 15699 | 21056 | 202 | EZY29472.1 | 15700 | 21057 | 308 | | | | |
| 1011 | 21058 | 483 | EZY31972.1 | 15702 | 21059 | 202 | EZY31973.1 | 15703 | 21060 | 308 | | | | |
| 1012 | 21061 | 483 | EZY36826.1 | 15705 | 21062 | 202 | EZY36827.1 | 15706 | 21063 | 308 | | | | |
| 1013 | 21064 | 483 | EZY36639.1 | 15708 | 21065 | 202 | EZY36640.1 | 15709 | 21066 | 308 | | | | |
| 1014 | 21067 | 483 | EZY38134.1 | 15711 | 21068 | 202 | EZY38135.1 | 15712 | 21069 | 308 | | | | |
| 1015 | 21070 | 483 | EZY43512.1 | 15714 | 21071 | 202 | EZY43513.1 | 15715 | 21072 | 308 | | | | |
| 1016 | 21073 | 375 | EZY44864.1 | 15717 | 21074 | 202 | EZY44865.1 | 15718 | 21075 | 308 | | | | |
| 1017 | 21076 | 483 | EZY46957.1 | 15720 | 21077 | 202 | EZY46958.1 | 15721 | 21078 | 308 | | | | |
| 1018 | 21079 | 483 | EZY51215.1 | 15723 | 21080 | 202 | EZY51216.1 | 15724 | 21081 | 308 | | | | |
| 1019 | 21082 | 375 | EZY52186.1 | 15726 | 21083 | 202 | EZY52187.1 | 15727 | 21084 | 308 | | | | |
| 1020 | 21085 | 375 | EZY54361.1 | 15729 | 21086 | 202 | EZY54362.1 | 15730 | 21087 | 308 | | | | |
| 1021 | 21088 | 375 | EZY58961.1 | 15732 | 21089 | 202 | EZY58962.1 | 15733 | 21090 | 308 | | | | |
| 1022 | 21091 | 483 | EZY61859.1 | 15735 | 21092 | 202 | EZY61860.1 | 15736 | 21093 | 308 | | | | |
| 1023 | 21094 | 483 | EZY66172.1 | 15738 | 21095 | 202 | EZY66173.1 | 15739 | 21096 | 308 | | | | |
| 1024 | 21097 | 483 | EZY70309.1 | 15741 | 21098 | 202 | EZY70310.1 | 15742 | 21099 | 308 | | | | |
| 1025 | 21100 | 483 | EZY70615.1 | 15744 | 21101 | 202 | EZY70616.1 | 15745 | 21102 | 308 | | | | |
| 1026 | 21103 | 483 | EZY75194.1 | 15747 | 21104 | 202 | EZY75195.1 | 15748 | 21105 | 308 | | | | |
| 1027 | 21106 | 483 | EZY78993.1 | 15750 | 21107 | 202 | EZY78994.1 | 15751 | 21108 | 308 | | | | |
| 1028 | 21109 | 483 | EZY77810.1 | 15753 | 21110 | 202 | EZY77811.1 | 15754 | 21111 | 308 | | | | |
| 1029 | 21112 | 483 | EZY80411.1 | 15756 | 21113 | 202 | EZY80412.1 | 15757 | 21114 | 308 | | | | |
| 1030 | 21115 | 483 | EZY85271.1 | 15759 | 21116 | 202 | EZY85272.1 | 15760 | 21117 | 308 | | | | |
| 1031 | 21118 | 483 | EZY90747.1 | 15762 | 21119 | 202 | EZY90748.1 | 15763 | 21120 | 308 | | | | |
| 1032 | 21121 | 483 | EZY86731.1 | 15765 | 21122 | 202 | EZY86732.1 | 15766 | 21123 | 308 | | | | |
| 1033 | 21124 | 483 | EZY93708.1 | 15768 | 21125 | 202 | EZY93709.1 | 15769 | 21126 | 308 | | | | |
| 1034 | 21127 | 483 | EZY96335.1 | 15771 | 21128 | 202 | EZY96336.1 | 15772 | 21129 | 308 | | | | |
| 1035 | 21130 | 232 | EZY99135.1 | 15774 | 21131 | 202 | EZY99136.1 | 15775 | 21132 | 308 | | | | |
| 1036 | 21133 | 483 | EZZ00575.1 | 15777 | 21134 | 202 | EZZ00576.1 | 15778 | 21135 | 308 | | | | |
| 1037 | 21136 | 232 | EZZ04413.1 | 15780 | 21137 | 202 | EZZ04414.1 | 15781 | 21138 | 308 | | | | |
| 1038 | 21139 | 483 | EZZ04095.1 | 15783 | 21140 | 202 | EZZ04096.1 | 15784 | 21141 | 308 | | | | |
| 1039 | 21142 | 375 | EZZ07025.1 | 15786 | 21143 | 202 | EZZ07026.1 | 15787 | 21144 | 308 | | | | |
| 1040 | 21145 | 483 | EZZ11500.1 | 15789 | 21146 | 202 | EZZ11501.1 | 15790 | 21147 | 308 | | | | |
| 1041 | 21148 | 483 | EZZ12220.1 | 15792 | 21149 | 202 | EZZ12221.1 | 15793 | 21150 | 165 | | | | |
| 1042 | 21151 | 483 | EZZ13374.1 | 15795 | 21152 | 202 | EZZ13375.1 | 15796 | 21153 | 308 | | | | |
| 1043 | 21154 | 483 | EZZ20015.1 | 15798 | 21155 | 202 | EZZ20016.1 | 15799 | 21156 | 261 | | | | |
| 1044 | 21157 | 483 | EZZ20491.1 | 15801 | 21158 | 202 | EZZ20492.1 | 15802 | 21159 | 308 | | | | |
| 1045 | 21160 | 483 | EZZ22877.1 | 15804 | 21161 | 202 | EZZ22878.1 | 15805 | 21162 | 308 | | | | |
| 1046 | 21163 | 483 | EZZ27502.1 | 15807 | 21164 | 202 | EZZ27503.1 | 15808 | 21165 | 308 | | | | |
| 1047 | 21166 | 483 | EZZ27417.1 | 15810 | 21167 | 202 | EZZ27418.1 | 15811 | 21168 | 308 | | | | |
| 1048 | 21169 | 483 | EZZ33551.1 | 15813 | 21170 | 202 | EZZ33552.1 | 15814 | 21171 | 308 | | | | |
| 1049 | 21172 | 483 | EZZ35197.1 | 15816 | 21173 | 202 | EZZ35198.1 | 15817 | 21174 | 308 | | | | |
| 1050 | 21175 | 483 | EZZ38881.1 | 15819 | 21176 | 202 | EZZ38882.1 | 15820 | 21177 | 308 | | | | |
| 1051 | 21178 | 483 | EZZ42312.1 | 15822 | 21179 | 202 | EZZ42313.1 | 15823 | 21180 | 308 | | | | |
| 1052 | 21181 | 483 | EZZ43688.1 | 15825 | 21182 | 202 | EZZ43689.1 | 15826 | 21183 | 308 | | | | |
| 1053 | 21184 | 483 | EZZ46491.1 | 15828 | 21185 | 202 | EZZ46492.1 | 15829 | 21186 | 308 | | | | |
| 1054 | 21187 | 483 | EZZ50761.1 | 15831 | 21188 | 202 | EZZ50762.1 | 15832 | 21189 | 308 | | | | |
| 1055 | 21190 | 483 | EZZ51035.1 | 15834 | 21191 | 202 | EZZ51036.1 | 15835 | 21192 | 308 | | | | |
| 1056 | 21193 | 483 | EZZ53436.1 | 15837 | 21194 | 202 | EZZ53437.1 | 15838 | 21195 | 308 | | | | |
| 1057 | 21196 | 483 | EZZ62214.1 | 15840 | 21197 | 202 | EZZ62215.1 | 15841 | 21198 | 308 | | | | |
| 1058 | 21199 | 483 | EZZ58807.1 | 15843 | 21200 | 202 | EZZ58808.1 | 15844 | 21201 | 308 | | | | |
| 1059 | 21202 | 483 | EZZ63775.1 | 15846 | 21203 | 202 | EZZ63776.1 | 15847 | 21204 | 308 | | | | |
| 1060 | 21205 | 375 | EZZ66973.1 | 15849 | 21206 | 202 | EZZ66974.1 | 15850 | 21207 | 308 | | | | |
| 1061 | 21208 | 483 | EZZ65103.1 | 15852 | 21209 | 202 | EZZ65104.1 | 15853 | 21210 | 308 | | | | |
| 1062 | 21211 | 483 | EZZ70712.1 | 15855 | 21212 | 202 | EZZ70713.1 | 15856 | 21213 | 308 | | | | |
| 1063 | 21214 | 483 | EZZ73601.1 | 15858 | 21215 | 202 | EZZ73602.1 | 15859 | 21216 | 308 | | | | |
| 1064 | 21217 | 483 | EZZ75112.1 | 15861 | 21218 | 202 | EZZ75113.1 | 15862 | 21219 | 308 | | | | |
| 1065 | 21220 | 483 | EZZ75509.1 | 15864 | 21221 | 202 | EZZ75510.1 | 15865 | 21222 | 308 | | | | |
| 1066 | 21223 | 483 | EZZ82798.1 | 15867 | 21224 | 202 | EZZ82799.1 | 15868 | 21225 | 308 | | | | |
| 1067 | 21226 | 483 | EZZ83621.1 | 15870 | 21227 | 202 | EZZ83622.1 | 15871 | 21228 | 308 | | | | |
| 1068 | 21229 | 483 | EZZ82653.1 | 15873 | 21230 | 202 | EZZ82654.1 | 15874 | 21231 | 308 | | | | |
| 1069 | 21232 | 483 | EZZ89013.1 | 15876 | 21233 | 202 | EZZ89014.1 | 15877 | 21234 | 308 | | | | |
| 1070 | 21235 | 483 | EZZ92960.1 | 15879 | 21236 | 202 | EZZ92961.1 | 15880 | 21237 | 308 | | | | |
| 1071 | 21238 | 483 | EZZ92682.1 | 15882 | 21239 | 202 | EZZ92683.1 | 15883 | 21240 | 308 | | | | |
| 1072 | 21241 | 483 | EZZ95682.1 | 15885 | 21242 | 202 | EZZ95683.1 | 15886 | 21243 | 308 | | | | |
| 1073 | 21244 | 483 | KAA05375.1 | 15888 | 21245 | 202 | KAA05376.1 | 15889 | 21246 | 308 | | | | |
| 1074 | 21247 | 483 | KAA05542.1 | 15891 | 21248 | 202 | KAA05543.1 | 15892 | 21249 | 308 | | | | |
| 1075 | 21250 | 483 | KAA05951.1 | 15894 | 21251 | 202 | KAA05952.1 | 15895 | 21252 | 308 | | | | |
| 1076 | 21253 | 483 | KAA12998.1 | 15897 | 21254 | 202 | KAA12999.1 | 15898 | 21255 | 308 | | | | |
| 1077 | 21256 | 483 | KAA12391.1 | 15900 | 21257 | 202 | KAA12392.1 | 15901 | 21258 | 308 | | | | |
| 1078 | 21259 | 483 | KAA13617.1 | 15903 | 21260 | 202 | KAA13618.1 | 15904 | 21261 | 308 | | | | |
| 1079 | 21262 | 483 | KAA22641.1 | 15906 | 21263 | 202 | KAA22642.1 | 15907 | 21264 | 308 | | | | |
| 1080 | 21265 | 483 | KAA21031.1 | 15909 | 21266 | 202 | KAA21032.1 | 15910 | 21267 | 308 | | | | |
| 1081 | 21268 | 483 | KAA21280.1 | 15912 | 21269 | 202 | KAA21281.1 | 15913 | 21270 | 308 | | | | |
| 1082 | 21271 | 483 | KAA28468.1 | 15915 | 21272 | 202 | KAA28469.1 | 15916 | 21273 | 308 | | | | |
| 1083 | 21274 | 483 | KAA29976.1 | 15918 | 21275 | 202 | KAA29977.1 | 15919 | 21276 | 308 | | | | |
| 1084 | 21277 | 483 | KAA28569.1 | 15921 | 21278 | 202 | KAA28570.1 | 15922 | 21279 | 308 | | | | |
| 1085 | 21280 | 483 | KAA35384.1 | 15924 | 21281 | 202 | KAA35385.1 | 15925 | 21282 | 308 | | | | |
| 1086 | 21283 | 483 | KAA38913.1 | 15927 | 21284 | 202 | KAA38914.1 | 15928 | 21285 | 308 | | | | |
| 1087 | 21286 | 483 | KAA39460.1 | 15930 | 21287 | 202 | KAA39461.1 | 15931 | 21288 | 308 | | | | |
| 1088 | 21289 | 483 | KAA42952.1 | 15933 | 21290 | 202 | KAA42953.1 | 15934 | 21291 | 308 | | | | |
| 1089 | 21292 | 483 | KAA45223.1 | 15936 | 21293 | 202 | KAA45224.1 | 15937 | 21294 | 308 | | | | |
| 1090 | 21295 | 483 | KAA46799.1 | 15939 | 21296 | 202 | KAA46800.1 | 15940 | 21297 | 308 | | | | |
| 1091 | 21298 | 483 | KAA49911.1 | 15942 | 21299 | 202 | KAA49912.1 | 15943 | 21300 | 308 | | | | |
| 1092 | 21301 | 483 | KAA51794.1 | 15945 | 21302 | 202 | KAA51795.1 | 15946 | 21303 | 308 | | | | |
| 1093 | 21304 | 483 | KAA59414.1 | 15948 | 21305 | 202 | KAA59415.1 | 15949 | 21306 | 308 | | | | |
| 1094 | 21307 | 483 | KAA58405.1 | 15951 | 21308 | 202 | KAA58406.1 | 15952 | 21309 | 308 | | | | |
| 1095 | 21310 | 483 | KAA61960.1 | 15954 | 21311 | 202 | KAA61961.1 | 15955 | 21312 | 308 | | | | |
| 1096 | 21313 | 483 | KAA63550.1 | 15957 | 21314 | 202 | KAA63551.1 | 15958 | 21315 | 308 | | | | |
| 1097 | 21316 | 483 | KAA66138.1 | 15960 | 21317 | 202 | KAA66139.1 | 15961 | 21318 | 308 | | | | |
| 1098 | 21319 | 483 | KAA68222.1 | 15963 | 21320 | 202 | KAA68223.1 | 15964 | 21321 | 308 | | | | |
| 1099 | 21322 | 483 | KAA73511.1 | 15966 | 21323 | 202 | KAA73512.1 | 15967 | 21324 | 308 | | | | |
| 1100 | 21325 | 483 | KAA74078.1 | 15969 | 21326 | 202 | KAA74079.1 | 15970 | 21327 | 308 | | | | |
| 1101 | 21328 | 483 | KAA76196.1 | 15972 | 21329 | 202 | KAA76197.1 | 15973 | 21330 | 308 | | | | |
| 1102 | 21331 | 483 | KAA79420.1 | 15975 | 21332 | 202 | KAA79421.1 | 15976 | 21333 | 308 | | | | |
| 1103 | 21334 | 483 | KAA82178.1 | 15978 | 21335 | 202 | KAA82179.1 | 15979 | 21336 | 308 | | | | |
| 1104 | 21337 | 483 | KAA82199.1 | 15981 | 21338 | 202 | KAA82200.1 | 15982 | 21339 | 308 | | | | |
| 1105 | 21340 | 483 | KAA86321.1 | 15984 | 21341 | 202 | KAA86322.1 | 15985 | 21342 | 308 | | | | |
| 1106 | 21343 | 483 | KAA89638.1 | 15987 | 21344 | 202 | KAA89639.1 | 15988 | 21345 | 308 | | | | |
| 1107 | 21346 | 483 | KAA93202.1 | 15990 | 21347 | 202 | KAA93203.1 | 15991 | 21348 | 308 | | | | |
| 1108 | 21349 | 483 | KAA93401.1 | 15993 | 21350 | 202 | KAA93402.1 | 15994 | 21351 | 308 | | | | |
| 1109 | 21352 | 483 | KAA96431.1 | 15996 | 21353 | 202 | KAA96432.1 | 15997 | 21354 | 308 | | | | |
| 1110 | 21355 | 483 | KAB00567.1 | 15999 | 21356 | 202 | KAB00568.1 | 16000 | 21357 | 308 | | | | |
| 1111 | 21358 | 483 | KAB03845.1 | 16002 | 21359 | 202 | KAB03846.1 | 16003 | 21360 | 308 | | | | |
| 1112 | 21361 | 483 | KAB06656.1 | 16005 | 21362 | 202 | KAB06657.1 | 16006 | 21363 | 308 | | | | |
| 1113 | 21364 | 483 | KAB08920.1 | 16008 | 21365 | 202 | KAB08921.1 | 16009 | 21366 | 308 | | | | |
| 1114 | 21367 | 483 | KAB10430.1 | 16011 | 21368 | 202 | KAB10431.1 | 16012 | 21369 | 308 | | | | |
| 1115 | 21370 | 483 | KAB11614.1 | 16014 | 21371 | 202 | KAB11615.1 | 16015 | 21372 | 308 | | | | |
| 1116 | 21373 | 483 | KAB16348.1 | 16017 | 21374 | 202 | KAB16349.1 | 16018 | 21375 | 308 | | | | |
| 1117 | 21376 | 483 | KAB18687.1 | 16020 | 21377 | 202 | KAB18688.1 | 16021 | 21378 | 308 | | | | |
| 1118 | 21379 | 483 | KAB19933.1 | 16023 | 21380 | 202 | KAB19934.1 | 16024 | 21381 | 308 | | | | |
| 1119 | 21382 | 483 | KAB24078.1 | 16026 | 21383 | 202 | KAB24079.1 | 16027 | 21384 | 308 | | | | |
| 1120 | 21385 | 483 | KAB29325.1 | 16029 | 21386 | 202 | KAB29326.1 | 16030 | 21387 | 308 | | | | |
| 1121 | 21388 | 483 | KAB29475.1 | 16032 | 21389 | 202 | KAB29476.1 | 16033 | 21390 | 308 | | | | |
| 1122 | 21391 | 483 | KAB32010.1 | 16035 | 21392 | 202 | KAB32011.1 | 16036 | 21393 | 308 | | | | |
| 1123 | 21394 | 483 | KAB33823.1 | 16038 | 21395 | 202 | KAB33824.1 | 16039 | 21396 | 308 | | | | |
| 1124 | 21397 | 483 | KAB37791.1 | 16041 | 21398 | 202 | KAB37792.1 | 16042 | 21399 | 308 | | | | |
| 1125 | 21400 | 483 | KAB38130.1 | 16044 | 21401 | 202 | KAB38131.1 | 16045 | 21402 | 308 | | | | |
| 1126 | 21403 | 483 | KAB42665.1 | 16047 | 21404 | 202 | KAB42666.1 | 16048 | 21405 | 308 | | | | |
| 1127 | 21406 | 483 | KAB46001.1 | 16050 | 21407 | 202 | KAB46002.1 | 16051 | 21408 | 308 | | | | |
| 1128 | 21409 | 483 | KAB45739.1 | 16053 | 21410 | 202 | KAB45740.1 | 16054 | 21411 | 308 | | | | |
| 1129 | 21412 | 483 | KAB51675.1 | 16056 | 21413 | 202 | KAB51676.1 | 16057 | 21414 | 308 | | | | |
| 1130 | 21415 | 483 | KAB53191.1 | 16059 | 21416 | 202 | KAB53192.1 | 16060 | 21417 | 308 | | | | |
| 1131 | 21418 | 483 | KAB55782.1 | 16062 | 21419 | 202 | KAB55783.1 | 16063 | 21420 | 308 | | | | |
| 1132 | 21421 | 483 | KAB58080.1 | 16065 | 21422 | 202 | KAB58081.1 | 16066 | 21423 | 308 | | | | |
| 1133 | 21424 | 483 | KAB59417.1 | 16068 | 21425 | 202 | KAB59418.1 | 16069 | 21426 | 308 | | | | |
| 1134 | 21427 | 131 | KAB62991.1 | 16071 | 21428 | 202 | KAB62992.1 | 16072 | 21429 | 308 | | | | |
| 1135 | 21430 | 483 | KAB66107.1 | 16074 | 21431 | 202 | KAB66108.1 | 16075 | 21432 | 308 | | | | |
| 1136 | 21433 | 131 | KAB66872.1 | 16077 | 21434 | 202 | KAB66873.1 | 16078 | 21435 | 308 | | | | |
| 1137 | 21436 | 483 | KAB70774.1 | 16080 | 21437 | 202 | KAB70775.1 | 16081 | 21438 | 308 | | | | |
| 1138 | 21439 | 483 | KAB73062.1 | 16083 | 21440 | 202 | KAB73063.1 | 16084 | 21441 | 308 | | | | |
| 1139 | 21442 | 483 | KAB75148.1 | 16086 | 21443 | 202 | KAB75149.1 | 16087 | 21444 | 308 | | | | |
| 1140 | 21445 | 483 | KAB78036.1 | 16089 | 21446 | 202 | KAB78037.1 | 16090 | 21447 | 308 | | | | |
| 1141 | 21448 | 483 | KAB81591.1 | 16092 | 21449 | 202 | KAB81592.1 | 16093 | 21450 | 308 | | | | |
| 1142 | 21451 | 483 | KAB84385.1 | 16095 | 21452 | 202 | KAB84386.1 | 16096 | 21453 | 308 | | | | |
| 1143 | 21454 | 483 | KAB84692.1 | 16098 | 21455 | 202 | KAB84693.1 | 16099 | 21456 | 308 | | | | |
| 1144 | 21457 | 483 | KAB91414.1 | 16101 | 21458 | 202 | KAB91415.1 | 16102 | 21459 | 308 | | | | |
| 1145 | 21460 | 483 | KAB92904.1 | 16104 | 21461 | 202 | KAB92905.1 | 16105 | 21462 | 308 | | | | |
| 1146 | 21463 | 483 | KAB93615.1 | 16107 | 21464 | 202 | KAB93616.1 | 16108 | 21465 | 308 | | | | |
| 1147 | 21466 | 483 | KAB98473.1 | 16110 | 21467 | 202 | KAB98474.1 | 16111 | 21468 | 308 | | | | |
| 1148 | 21469 | 483 | KAB98434.1 | 16113 | 21470 | 202 | KAB98435.1 | 16114 | 21471 | 308 | | | | |
| 1149 | 21472 | 483 | KAC00307.1 | 16116 | 21473 | 202 | KAC00308.1 | 16117 | 21474 | 308 | | | | |
| 1150 | 21475 | 483 | KAC04035.1 | 16119 | 21476 | 202 | KAC04036.1 | 16120 | 21477 | 308 | | | | |
| 1151 | 21478 | 483 | KAC08056.1 | 16122 | 21479 | 202 | KAC08057.1 | 16123 | 21480 | 308 | | | | |
| 1152 | 21481 | 483 | KAC10253.1 | 16125 | 21482 | 202 | KAC10254.1 | 16126 | 21483 | 308 | | | | |
| 1153 | 21484 | 483 | KAC11814.1 | 16128 | 21485 | 202 | KAC11815.1 | 16129 | 21486 | 308 | | | | |
| 1154 | 21487 | 483 | KAC14779.1 | 16131 | 21488 | 202 | KAC14780.1 | 16132 | 21489 | 308 | | | | |
| 1155 | 21490 | 483 | KAC17656.1 | 16134 | 21491 | 202 | KAC17657.1 | 16135 | 21492 | 308 | | | | |
| 1156 | 21493 | 131 | KAC20375.1 | 16137 | 21494 | 202 | KAC20376.1 | 16138 | 21495 | 308 | | | | |
| 1157 | 21496 | 483 | KAC21944.1 | 16140 | 21497 | 202 | KAC21945.1 | 16141 | 21498 | 308 | | | | |
| 1158 | 21499 | 483 | KAC25276.1 | 16143 | 21500 | 202 | KAC25277.1 | 16144 | 21501 | 308 | | | | |
| 1159 | 21502 | 483 | KAC29149.1 | 16146 | 21503 | 202 | KAC29150.1 | 16147 | 21504 | 308 | | | | |
| 1160 | 21505 | 483 | KAC33552.1 | 16149 | 21506 | 202 | KAC33553.1 | 16150 | 21507 | 308 | | | | |
| 1161 | 21508 | 483 | KAC34155.1 | 16152 | 21509 | 202 | KAC34156.1 | 16153 | 21510 | 308 | | | | |
| 1162 | 21511 | 131 | KAC33239.1 | 16155 | 21512 | 202 | KAC33240.1 | 16156 | 21513 | 308 | | | | |
| 1163 | 21514 | 483 | KAC37670.1 | 16158 | 21515 | 202 | KAC37671.1 | 16159 | 21516 | 308 | | | | |
| 1164 | 21517 | 131 | KAC40878.1 | 16161 | 21518 | 202 | KAC40879.1 | 16162 | 21519 | 308 | | | | |
| 1165 | 21520 | 483 | KAC45124.1 | 16164 | 21521 | 202 | KAC45125.1 | 16165 | 21522 | 308 | | | | |
| 1166 | 21523 | 483 | KAC44747.1 | 16167 | 21524 | 202 | KAC44748.1 | 16168 | 21525 | 308 | | | | |
| 1167 | 21526 | 483 | KAC51537.1 | 16170 | 21527 | 202 | KAC51538.1 | 16171 | 21528 | 308 | | | | |
| 1168 | 21529 | 483 | KAC50573.1 | 16173 | 21530 | 202 | KAC50574.1 | 16174 | 21531 | 308 | | | | |
| 1169 | 21532 | 483 | KAC52442.1 | 16176 | 21533 | 202 | KAC52443.1 | 16177 | 21534 | 308 | | | | |
| 1170 | 21535 | 483 | KAC57901.1 | 16179 | 21536 | 202 | KAC5 7902.1 | 16180 | 21537 | 308 | | | | |
| 1171 | 21538 | 483 | KAC59265.1 | 16182 | 21539 | 202 | KAC59266.1 | 16183 | 21540 | 308 | | | | |
| 1172 | 21541 | 483 | KAC64900.1 | 16185 | 21542 | 202 | KAC64901.1 | 16186 | 21543 | 308 | | | | |
| 1173 | 21544 | 483 | KAC65214.1 | 16188 | 21545 | 202 | KAC65215.1 | 16189 | 21546 | 308 | | | | |
| 1174 | 21547 | 483 | KAC66577.1 | 16191 | 21548 | 202 | KAC66578.1 | 16192 | 21549 | 308 | | | | |
| 1175 | 21550 | 483 | KAC76249.1 | 16194 | 21551 | 202 | KAC76250.1 | 16195 | 21552 | 308 | | | | |
| 1176 | 21553 | 483 | KAC70674.1 | 16197 | 21554 | 202 | KAC70675.1 | 16198 | 21555 | 308 | | | | |
| 1177 | 21556 | 131 | KAC73888.1 | 16200 | 21557 | 202 | KAC73889.1 | 16201 | 21558 | 308 | | | | |
| 1178 | 21559 | 131 | KAC78735.1 | 16203 | 21560 | 202 | KAC78736.1 | 16204 | 21561 | 308 | | | | |
| 1179 | 21562 | 483 | KAC82510.1 | 16206 | 21563 | 202 | KAC82511.1 | 16207 | 21564 | 308 | | | | |
| 1180 | 21565 | 483 | KAC80330.1 | 16209 | 21566 | 202 | KAC80331.1 | 16210 | 21567 | 308 | | | | |
| 1181 | 21568 | 483 | KAC86157.1 | 16212 | 21569 | 202 | KAC86158.1 | 16213 | 21570 | 308 | | | | |
| 1182 | 21571 | 483 | KAC89252.1 | 16215 | 21572 | 202 | KAC89253.1 | 16216 | 21573 | 308 | | | | |
| 1183 | 21574 | 483 | KAC89179.1 | 16218 | 21575 | 202 | KAC89180.1 | 16219 | 21576 | 308 | | | | |
| 1184 | 21577 | 483 | KAC93842.1 | 16221 | 21578 | 202 | KAC93843.1 | 16222 | 21579 | 308 | | | | |
| 1185 | 21580 | 483 | KAC96628.1 | 16224 | 21581 | 202 | KAC96629.1 | 16225 | 21582 | 308 | | | | |
| 1186 | 21583 | 483 | KAC97651.1 | 16227 | 21584 | 202 | KAC97652.1 | 16228 | 21585 | 308 | | | | |
| 1187 | 21586 | 483 | KAD00062.1 | 16230 | 21587 | 202 | KAD00063.1 | 16231 | 21588 | 308 | | | | |
| 1188 | 21589 | 483 | KAD04304.1 | 16233 | 21590 | 202 | KAD04305.1 | 16234 | 21591 | 308 | | | | |
| 1189 | 21592 | 483 | KAD08150.1 | 16236 | 21593 | 202 | KAD08151.1 | 16237 | 21594 | 308 | | | | |
| 1190 | 21595 | 483 | KAD07873.1 | 16239 | 21596 | 202 | KAD07874.1 | 16240 | 21597 | 308 | | | | |
| 1191 | 21598 | 483 | KAD11426.1 | 16242 | 21599 | 202 | KAD11427.1 | 16243 | 21600 | 308 | | | | |
| 1192 | 21601 | 483 | KAD14194.1 | 16245 | 21602 | 202 | KAD14195.1 | 16246 | 21603 | 308 | | | | |
| 1193 | 21604 | 483 | KAD16931.1 | 16248 | 21605 | 202 | KAD16932.1 | 16249 | 21606 | 308 | | | | |
| 1194 | 21607 | 483 | KAD20110.1 | 16251 | 21608 | 202 | KAD20111.1 | 16252 | 21609 | 308 | | | | |
| 1195 | 21610 | 483 | KAD22450.1 | 16254 | 21611 | 202 | KAD22451.1 | 16255 | 21612 | 308 | | | | |
| 1196 | 21613 | 483 | KAD23851.1 | 16257 | 21614 | 202 | KAD23852.1 | 16258 | 21615 | 308 | | | | |
| 1197 | 21616 | 483 | KAD27440.1 | 16260 | 21617 | 202 | KAD27441.1 | 16261 | 21618 | 308 | | | | |
| 1198 | 21619 | 483 | KAD29133.1 | 16263 | 21620 | 202 | KAD29134.1 | 16264 | 21621 | 308 | | | | |
| 1199 | 21622 | 483 | KAD33181.1 | 16266 | 21623 | 202 | KAD33182.1 | 16267 | 21624 | 308 | | | | |
| 1200 | 21625 | 483 | KAD35156.1 | 16269 | 21626 | 202 | KAD35157.1 | 16270 | 21627 | 308 | | | | |
| 1201 | 21628 | 483 | KAD41137.1 | 16272 | 21629 | 202 | KAD41138.1 | 16273 | 21630 | 308 | | | | |
| 1202 | 21631 | 483 | KAD37495.1 | 16275 | 21632 | 202 | KAD37496.1 | 16276 | 21633 | 308 | | | | |
| 1203 | 21634 | 483 | KAD41936.1 | 16278 | 21635 | 202 | KAD41937.1 | 16279 | 21636 | 308 | | | | |
| 1204 | 21637 | 483 | KAD44773.1 | 16281 | 21638 | 202 | KAD44774.1 | 16282 | 21639 | 308 | | | | |
| 1205 | 21640 | 483 | KAD49430.1 | 16284 | 21641 | 202 | KAD49431.1 | 16285 | 21642 | 308 | | | | |
| 1206 | 21643 | 483 | KAD48761.1 | 16287 | 21644 | 202 | KAD48762.1 | 16288 | 21645 | 308 | | | | |
| 1207 | 21646 | 483 | KAD53923.1 | 16290 | 21647 | 202 | KAD53924.1 | 16291 | 21648 | 308 | | | | |
| 1208 | 21649 | 483 | KAD57263.1 | 16293 | 21650 | 202 | KAD57264.1 | 16294 | 21651 | 308 | | | | |
| 1209 | 21652 | 483 | KAD59396.1 | 16296 | 21653 | 202 | KAD59397.1 | 16297 | 21654 | 308 | | | | |
| 1210 | 21655 | 483 | KAD59588.1 | 16299 | 21656 | 202 | KAD59589.1 | 16300 | 21657 | 308 | | | | |
| 1211 | 21658 | 483 | KAD64093.1 | 16302 | 21659 | 202 | KAD64094.1 | 16303 | 21660 | 308 | | | | |
| 1212 | 21661 | 483 | KAD64969.1 | 16305 | 21662 | 202 | KAD64970.1 | 16306 | 21663 | 308 | | | | |
| 1213 | 21664 | 483 | KAD71130.1 | 16308 | 21665 | 202 | KAD71131.1 | 16309 | 21666 | 308 | | | | |
| 1214 | 21667 | 483 | KAD76142.1 | 16311 | 21668 | 202 | KAD76143.1 | 16312 | 21669 | 308 | | | | |
| 1215 | 21670 | 483 | KAD70467.1 | 16314 | 21671 | 202 | KAD70468.1 | 16315 | 21672 | 308 | | | | |
| 1216 | 21673 | 483 | KAD78843.1 | 16317 | 21674 | 202 | KAD78844.1 | 16318 | 21675 | 308 | | | | |
| 1217 | 21676 | 483 | KAD77328.1 | 16320 | 21677 | 202 | KAD77329.1 | 16321 | 21678 | 308 | | | | |
| 1218 | 21679 | 483 | KAD80655.1 | 16323 | 21680 | 202 | KAD80656.1 | 16324 | 21681 | 308 | | | | |
| 1219 | 21682 | 483 | KAD85220.1 | 16326 | 21683 | 202 | KAD85221.1 | 16327 | 21684 | 308 | | | | |
| 1220 | 21685 | 483 | KAD85660.1 | 16329 | 21686 | 202 | KAD85661.1 | 16330 | 21687 | 308 | | | | |
| 1221 | 21688 | 483 | KAD90245.1 | 16332 | 21689 | 202 | KAD90246.1 | 16333 | 21690 | 308 | | | | |
| 1222 | 21691 | 483 | KAD93692.1 | 16335 | 21692 | 202 | KAD93693.1 | 16336 | 21693 | 308 | | | | |
| 1223 | 21694 | 483 | KAD94379.1 | 16338 | 21695 | 202 | KAD94380.1 | 16339 | 21696 | 308 | | | | |
| 1224 | 21697 | 483 | KAD99174.1 | 16341 | 21698 | 202 | KAD99175.1 | 16342 | 21699 | 308 | | | | |
| 1225 | 21700 | 483 | KAD98840.1 | 16344 | 21701 | 202 | KAD98841.1 | 16345 | 21702 | 308 | | | | |
| 1226 | 21703 | 131 | KAD99161.1 | 16347 | 21704 | 202 | KAD99162.1 | 16348 | 21705 | 308 | | | | |
| 1227 | 21706 | 131 | KAE03355.1 | 16350 | 21707 | 202 | KAE03356.1 | 16351 | 21708 | 308 | | | | |
| 1228 | 21709 | 483 | KAE06089.1 | 16353 | 21710 | 202 | KAE06090.1 | 16354 | 21711 | 308 | | | | |
| 1229 | 21712 | 483 | KAE10341.1 | 16356 | 21713 | 202 | KAE10342.1 | 16357 | 21714 | 308 | | | | |
| 1230 | 21715 | 483 | KAE11830.1 | 16359 | 21716 | 202 | KAE11831.1 | 16360 | 21717 | 308 | | | | |
| 1231 | 21718 | 483 | KAE16056.1 | 16362 | 21719 | 202 | KAE16057.1 | 16363 | 21720 | 308 | | | | |
| 1232 | 21721 | 483 | KAE17678.1 | 16365 | 21722 | 202 | KAE17679.1 | 16366 | 21723 | 308 | | | | |
| 1233 | 21724 | 483 | KAE21312.1 | 16368 | 21725 | 202 | KAE21313.1 | 16369 | 21726 | 308 | | | | |
| 1234 | 21727 | 483 | KAE22704.1 | 16371 | 21728 | 202 | KAE22705.1 | 16372 | 21729 | 308 | | | | |
| 1235 | 21730 | 483 | KAE26216.1 | 16374 | 21731 | 202 | KAE26217.1 | 16375 | 21732 | 308 | | | | |
| 1236 | 21733 | 483 | KAE28379.1 | 16377 | 21734 | 202 | KAE28380.1 | 16378 | 21735 | 308 | | | | |
| 1237 | 21736 | 483 | KAE30965.1 | 16380 | 21737 | 202 | KAE30966.1 | 16381 | 21738 | 308 | | | | |
| 1238 | 21739 | 483 | KAE34714.1 | 16383 | 21740 | 202 | KAE34715.1 | 16384 | 21741 | 308 | | | | |
| 1239 | 21742 | 483 | KAE36915.1 | 16386 | 21743 | 202 | KAE36916.1 | 16387 | 21744 | 308 | | | | |
| 1240 | 21745 | 483 | KAE38767.1 | 16389 | 21746 | 202 | KAE38768.1 | 16390 | 21747 | 308 | | | | |
| 1241 | 21748 | 483 | KAE42474.1 | 16392 | 21749 | 202 | KAE42475.1 | 16393 | 21750 | 308 | | | | |
| 1242 | 21751 | 483 | KAE45133.1 | 16395 | 21752 | 202 | KAE45134.1 | 16396 | 21753 | 308 | | | | |
| 1243 | 21754 | 131 | KAE45864.1 | 16398 | 21755 | 202 | KAE45865.1 | 16399 | 21756 | 308 | | | | |
| 1244 | 21757 | 131 | KAE50289.1 | 16401 | 21758 | 202 | KAE50290.1 | 16402 | 21759 | 308 | | | | |
| 1245 | 21760 | 483 | KAE51869.1 | 16404 | 21761 | 202 | KAE51870.1 | 16405 | 21762 | 308 | | | | |
| 1246 | 21763 | 483 | KAE56031.1 | 16407 | 21764 | 202 | KAE56032.1 | 16408 | 21765 | 308 | | | | |
| 1247 | 21766 | 483 | KAE58617.1 | 16410 | 21767 | 202 | KAE58618.1 | 16411 | 21768 | 308 | | | | |
| 1248 | 21769 | 483 | KAE60713.1 | 16413 | 21770 | 202 | KAE60714.1 | 16414 | 21771 | 308 | | | | |
| 1249 | 21772 | 483 | KAE63795.1 | 16416 | 21773 | 202 | KAE63796.1 | 16417 | 21774 | 308 | | | | |
| 1250 | 21775 | 483 | KAE63592.1 | 16419 | 21776 | 202 | KAE63593.1 | 16420 | 21777 | 308 | | | | |
| 1251 | 21778 | 483 | KAE68674.1 | 16422 | 21779 | 202 | KAE68675.1 | 16423 | 21780 | 308 | | | | |
| 1252 | 21781 | 483 | KAE69668.1 | 16425 | 21782 | 202 | KAE69669.1 | 16426 | 21783 | 308 | | | | |
| 1253 | 21784 | 483 | KAE72489.1 | 16428 | 21785 | 202 | KAE72490.1 | 16429 | 21786 | 308 | | | | |
| 1254 | 21787 | 483 | KAE78482.1 | 16431 | 21788 | 202 | KAE78483.1 | 16432 | 21789 | 308 | | | | |
| 1255 | 21790 | 483 | KAE76734.1 | 16434 | 21791 | 202 | KAE76735.1 | 16435 | 21792 | 308 | | | | |
| 1256 | 21793 | 483 | KAE81549.1 | 16437 | 21794 | 202 | KAE81550.1 | 16438 | 21795 | 308 | | | | |
| 1257 | 21796 | 483 | KAE84895.1 | 16440 | 21797 | 202 | KAE84896.1 | 16441 | 21798 | 308 | | | | |
| 1258 | 21799 | 483 | KAE84989.1 | 16443 | 21800 | 202 | KAE84990.1 | 16444 | 21801 | 308 | | | | |
| 1259 | 21802 | 483 | KAE89402.1 | 16446 | 21803 | 202 | KAE89403.1 | 16447 | 21804 | 308 | | | | |
| 1260 | 21805 | 483 | KAE90294.1 | 16449 | 21806 | 202 | KAE90295.1 | 16450 | 21807 | 308 | | | | |
| 1261 | 21808 | 483 | KAE93400.1 | 16452 | 21809 | 202 | KAE93401.1 | 16453 | 21810 | 308 | | | | |
| 1262 | 21811 | 483 | KAE97231.1 | 16455 | 21812 | 202 | KAE97232.1 | 16456 | 21813 | 308 | | | | |
| 1263 | 21814 | 483 | KAE99812.1 | 16458 | 21815 | 202 | KAE99813.1 | 16459 | 21816 | 308 | | | | |
| 1264 | 21817 | 483 | KAE99885.1 | 16461 | 21818 | 202 | KAE99886.1 | 16462 | 21819 | 308 | | | | |
| 1265 | 21820 | 483 | KAF04814.1 | 16464 | 21821 | 202 | KAF04815.1 | 16465 | 21822 | 308 | | | | |
| 1266 | 21823 | 483 | KAF09787.1 | 16467 | 21824 | 202 | KAF09788.1 | 16468 | 21825 | 308 | | | | |
| 1267 | 21826 | 483 | KAF06946.1 | 16470 | 21827 | 202 | KAF06947.1 | 16471 | 21828 | 308 | | | | |
| 1268 | 21829 | 483 | KAF12147.1 | 16473 | 21830 | 202 | KAF12148.1 | 16474 | 21831 | 308 | | | | |
| 1269 | 21832 | 375 | KAF14359.1 | 16476 | 21833 | 202 | KAF14360.1 | 16477 | 21834 | 308 | | | | |
| 1270 | 21835 | 483 | KAF17948.1 | 16479 | 21836 | 202 | KAF17949.1 | 16480 | 21837 | 308 | | | | |
| 1271 | 21838 | 483 | KAF21047.1 | 16482 | 21839 | 202 | KAF21048.1 | 16483 | 21840 | 308 | | | | |
| 1272 | 21841 | 483 | KAF20974.1 | 16485 | 21842 | 202 | KAF20975.1 | 16486 | 21843 | 308 | | | | |
| 1273 | 21844 | 483 | KAF25641.1 | 16488 | 21845 | 202 | KAF25642.1 | 16489 | 21846 | 308 | | | | |
| 1274 | 21847 | 483 | KAF31106.1 | 16491 | 21848 | 202 | KAF31107.1 | 16492 | 21849 | 308 | | | | |
| 1275 | 21850 | 483 | KAF28012.1 | 16494 | 21851 | 202 | KAF28013.1 | 16495 | 21852 | 308 | | | | |
| 1276 | 21853 | 483 | KAF33772.1 | 16497 | 21854 | 202 | KAF33773.1 | 16498 | 21855 | 308 | | | | |
| 1277 | 21856 | 483 | KAF35728.1 | 16500 | 21857 | 202 | KAF35729.1 | 16501 | 21858 | 308 | | | | |
| 1278 | 21859 | 483 | KAF38653.1 | 16503 | 21860 | 202 | KAF38654.1 | 16504 | 21861 | 308 | | | | |
| 1279 | 21862 | 483 | KAF42880.1 | 16506 | 21863 | 202 | KAF42881.1 | 16507 | 21864 | 308 | | | | |
| 1280 | 21865 | 483 | KAF42844.1 | 16509 | 21866 | 202 | KAF42845.1 | 16510 | 21867 | 308 | | | | |
| 1281 | 21868 | 483 | KAF46907.1 | 16512 | 21869 | 202 | KAF46908.1 | 16513 | 21870 | 308 | | | | |
| 1282 | 21871 | 483 | KAF47441.1 | 16515 | 21872 | 202 | KAF47442.1 | 16516 | 21873 | 308 | | | | |
| 1283 | 21874 | 131 | KAF47998.1 | 16518 | 21875 | 202 | KAF47999.1 | 16519 | 21876 | 308 | | | | |
| 1284 | 21877 | 483 | KAF54047.1 | 16521 | 21878 | 202 | KAF54048.1 | 16522 | 21879 | 308 | | | | |
| 1285 | 21880 | 483 | KAF54412.1 | 16524 | 21881 | 202 | KAF54413.1 | 16525 | 21882 | 308 | | | | |
| 1286 | 21883 | 483 | KAF60184.1 | 16527 | 21884 | 202 | KAF60185.1 | 16528 | 21885 | 308 | | | | |
| 1287 | 21886 | 483 | KAF62545.1 | 16530 | 21887 | 202 | KAF62546.1 | 16531 | 21888 | 308 | | | | |
| 1288 | 21889 | 483 | KAF64110.1 | 16533 | 21890 | 202 | KAF64111.1 | 16534 | 21891 | 308 | | | | |
| 1289 | 21892 | 483 | KAF67902.1 | 16536 | 21893 | 202 | KAF67903.1 | 16537 | 21894 | 308 | | | | |
| 1290 | 21895 | 483 | KAF70711.1 | 16539 | 21896 | 202 | KAF70712.1 | 16540 | 21897 | 308 | | | | |
| 1291 | 21898 | 483 | KAF71813.1 | 16542 | 21899 | 202 | KAF71814.1 | 16543 | 21900 | 308 | | | | |
| 1292 | 21901 | 483 | KAF75588.1 | 16545 | 21902 | 202 | KAF75589.1 | 16546 | 21903 | 308 | | | | |
| 1293 | 21904 | 483 | KAF76889.1 | 16548 | 21905 | 202 | KAF76890.1 | 16549 | 21906 | 308 | | | | |
| 1294 | 21907 | 483 | KAF80457.1 | 16551 | 21908 | 202 | KAF80458.1 | 16552 | 21909 | 308 | | | | |
| 1295 | 21910 | 483 | KAF84646.1 | 16554 | 21911 | 202 | KAF84647.1 | 16555 | 21912 | 308 | | | | |
| 1296 | 21913 | 483 | KAF88484.1 | 16557 | 21914 | 202 | KAF88485.1 | 16558 | 21915 | 308 | | | | |
| 1297 | 21916 | 483 | KAF92872.1 | 16560 | 21917 | 202 | KAF92873.1 | 16561 | 21918 | 308 | | | | |
| 1298 | 21919 | 483 | KAF91776.1 | 16563 | 21920 | 202 | KAF91777.1 | 16564 | 21921 | 308 | | | | |
| 1299 | 21922 | 483 | KAG01059.1 | 16566 | 21923 | 202 | KAG01060.1 | 16567 | 21924 | 308 | | | | |
| 1300 | 21925 | 131 | KAF98700.1 | 16569 | 21926 | 202 | KAF98701.1 | 16570 | 21927 | 308 | | | | |
| 1301 | 21928 | 131 | KAF97454.1 | 16572 | 21929 | 202 | KAF97455.1 | 16573 | 21930 | 308 | | | | |
| 1302 | 21931 | 483 | KAG04153.1 | 16575 | 21932 | 202 | KAG04154.1 | 16576 | 21933 | 308 | | | | |
| 1303 | 21934 | 483 | KAG04818.1 | 16578 | 21935 | 202 | KAG04819.1 | 16579 | 21936 | 308 | | | | |
| 1304 | 21937 | 483 | KAG08554.1 | 16581 | 21938 | 202 | KAG08555.1 | 16582 | 21939 | 308 | | | | |
| 1305 | 21940 | 483 | KAG10679.1 | 16584 | 21941 | 202 | KAG10680.1 | 16585 | 21942 | 308 | | | | |
| 1306 | 21943 | 483 | KAG15999.1 | 16587 | 21944 | 202 | KAG16000.1 | 16588 | 21945 | 308 | | | | |
| 1307 | 21946 | 483 | KAG21724.1 | 16590 | 21947 | 202 | KAG21725.1 | 16591 | 21948 | 308 | | | | |
| 1308 | 21949 | 483 | KAG29028.1 | 16593 | 21950 | 202 | KAG29029.1 | 16594 | 21951 | 308 | | | | |
| 1309 | 21952 | 483 | KAG32268.1 | 16596 | 21953 | 202 | KAG32269.1 | 16597 | 21954 | 308 | | | | |
| 1310 | 21955 | 483 | KAG34709.1 | 16599 | 21956 | 202 | KAG34710.1 | 16600 | 21957 | 308 | | | | |
| 1311 | 21958 | 483 | KAG37985.1 | 16602 | 21959 | 202 | KAG37986.1 | 16603 | 21960 | 308 | | | | |
| 1312 | 21961 | 483 | KAG51660.1 | 16605 | 21962 | 202 | KAG51661.1 | 16606 | 21963 | 308 | | | | |
| 1313 | 21964 | 483 | KAG55491.1 | 16608 | 21965 | 202 | KAG55492.1 | 16609 | 21966 | 308 | | | | |
| 1314 | 21967 | 483 | KAG59144.1 | 16611 | 21968 | 202 | KAG59145.1 | 16612 | 21969 | 308 | | | | |
| 1315 | 21970 | 483 | KAG59706.1 | 16614 | 21971 | 202 | KAG59707.1 | 16615 | 21972 | 308 | | | | |
| 1316 | 21973 | 483 | KAG65357.1 | 16617 | 21974 | 202 | KAG65358.1 | 16618 | 21975 | 308 | | | | |
| 1317 | 21976 | 483 | KAG67526.1 | 16620 | 21977 | 202 | KAG67527.1 | 16621 | 21978 | 308 | | | | |
| 1318 | 21979 | 483 | KAG68213.1 | 16623 | 21980 | 202 | KAG68214.1 | 16624 | 21981 | 308 | | | | |
| 1319 | 21982 | 483 | KAG69691.1 | 16626 | 21983 | 202 | KAG69692.1 | 16627 | 21984 | 308 | | | | |
| 1320 | 21985 | 483 | KAG74734.1 | 16629 | 21986 | 202 | KAG74735.1 | 16630 | 21987 | 308 | | | | |
| 1321 | 21988 | 483 | KAG76837.1 | 16632 | 21989 | 202 | KAG76838.1 | 16633 | 21990 | 308 | | | | |
| 1322 | 21991 | 483 | KAG76106.1 | 16635 | 21992 | 202 | KAG76107.1 | 16636 | 21993 | 308 | | | | |
| 1323 | 21994 | 483 | KAG81174.1 | 16638 | 21995 | 202 | KAG81175.1 | 16639 | 21996 | 308 | | | | |
| 1324 | 21997 | 483 | KAG82428.1 | 16641 | 21998 | 202 | KAG82429.1 | 16642 | 21999 | 308 | | | | |
| 1325 | 22000 | 483 | KAG84390.1 | 16644 | 22001 | 202 | KAG84391.1 | 16645 | 22002 | 308 | | | | |
| 1326 | 22003 | 483 | KAG88851.1 | 16647 | 22004 | 202 | KAG88852.1 | 16648 | 22005 | 308 | | | | |
| 1327 | 22006 | 483 | KAG92814.1 | 16650 | 22007 | 202 | KAG92815.1 | 16651 | 22008 | 308 | | | | |
| 1328 | 22009 | 483 | KAG95313.1 | 16653 | 22010 | 202 | KAG95314.1 | 16654 | 22011 | 308 | | | | |
| 1329 | 22012 | 483 | KAH01281.1 | 16656 | 22013 | 202 | KAH01282.1 | 16657 | 22014 | 308 | | | | |
| 1330 | 22015 | 483 | KAH06867.1 | 16659 | 22016 | 202 | KAH06868.1 | 16660 | 22017 | 308 | | | | |
| 1331 | 22018 | 483 | KAH07512.1 | 16662 | 22019 | 202 | KAH07513.1 | 16663 | 22020 | 308 | | | | |
| 1332 | 22021 | 483 | KAH16051.1 | 16665 | 22022 | 202 | KAH16052.1 | 16666 | 22023 | 308 | | | | |
| 1333 | 22024 | 483 | KAH15236.1 | 16668 | 22025 | 202 | KAH15237.1 | 16669 | 22026 | 308 | | | | |
| 1334 | 22027 | 483 | KAH19615.1 | 16671 | 22028 | 202 | KAH19616.1 | 16672 | 22029 | 308 | | | | |
| 1335 | 22030 | 483 | KAH24555.1 | 16674 | 22031 | 202 | KAH24556.1 | 16675 | 22032 | 308 | | | | |
| 1336 | 22033 | 483 | KAH28156.1 | 16677 | 22034 | 202 | KAH28157.1 | 16678 | 22035 | 308 | | | | |
| 1337 | 22036 | 483 | KAH29213.1 | 16680 | 22037 | 202 | KAH29214.1 | 16681 | 22038 | 308 | | | | |
| 1338 | 22039 | 483 | KAH33222.1 | 16683 | 22040 | 202 | KAH33223.1 | 16684 | 22041 | 308 | | | | |
| 1339 | 22042 | 483 | KAH35671.1 | 16686 | 22043 | 202 | KAH35672.1 | 16687 | 22044 | 308 | | | | |
| 1340 | 22045 | 483 | KAH37680.1 | 16689 | 22046 | 202 | KAH37681.1 | 16690 | 22047 | 308 | | | | |
| 1341 | 22048 | 483 | KAH40128.1 | 16692 | 22049 | 202 | KAH40129.1 | 16693 | 22050 | 308 | | | | |
| 1342 | 22051 | 483 | KAH41310.1 | 16695 | 22052 | 202 | KAH41311.1 | 16696 | 22053 | 308 | | | | |
| 1343 | 22054 | 483 | KAH45886.1 | 16698 | 22055 | 202 | KAH45887.1 | 16699 | 22056 | 308 | | | | |
| 1344 | 22057 | 483 | KAH47352.1 | 16701 | 22058 | 202 | KAH47353.1 | 16702 | 22059 | 308 | | | | |
| 1345 | 22060 | 483 | KAH50034.1 | 16704 | 22061 | 202 | KAH50035.1 | 16705 | 22062 | 308 | | | | |
| 1346 | 22063 | 483 | KAH53956.1 | 16707 | 22064 | 202 | KAH53957.1 | 16708 | 22065 | 308 | | | | |
| 1347 | 22066 | 483 | KAH55296.1 | 16710 | 22067 | 202 | KAH55297.1 | 16711 | 22068 | 308 | | | | |
| 1348 | 22069 | 483 | KAH59145.1 | 16713 | 22070 | 202 | KAH59146.1 | 16714 | 22071 | 308 | | | | |
| 1349 | 22072 | 483 | KAH62177.1 | 16716 | 22073 | 202 | KAH62178.1 | 16717 | 22074 | 308 | | | | |
| 1350 | 22075 | 483 | KAH64793.1 | 16719 | 22076 | 202 | KAH64794.1 | 16720 | 22077 | 308 | | | | |
| 1351 | 22078 | 483 | KAH67713.1 | 16722 | 22079 | 202 | KAH67714.1 | 16723 | 22080 | 308 | | | | |
| 1352 | 22081 | 483 | KAH70234.1 | 16725 | 22082 | 202 | KAH70235.1 | 16726 | 22083 | 308 | | | | |
| 1353 | 22084 | 483 | KAH70951.1 | 16728 | 22085 | 202 | KAH70952.1 | 16729 | 22086 | 308 | | | | |
| 1354 | 22087 | 483 | KAH74940.1 | 16731 | 22088 | 202 | KAH74941.1 | 16732 | 22089 | 308 | | | | |
| 1355 | 22090 | 483 | KAH77219.1 | 16734 | 22091 | 202 | KAH77220.1 | 16735 | 22092 | 308 | | | | |
| 1356 | 22093 | 483 | KAH77146.1 | 16737 | 22094 | 202 | KAH77147.1 | 16738 | 22095 | 308 | | | | |
| 1357 | 22096 | 483 | KAH82034.1 | 16740 | 22097 | 202 | KAH82035.1 | 16741 | 22098 | 308 | | | | |
| 1358 | 22099 | 131 | KAH85415.1 | 16743 | 22100 | 202 | KAH85416.1 | 16744 | 22101 | 308 | | | | |
| 1359 | 22102 | 483 | KAH87603.1 | 16746 | 22103 | 202 | KAH87604.1 | 16747 | 22104 | 308 | | | | |
| 1360 | 22105 | 483 | KAH91092.1 | 16749 | 22106 | 202 | KAH91093.1 | 16750 | 22107 | 308 | | | | |
| 1361 | 22108 | 483 | KAH91683.1 | 16752 | 22109 | 202 | KAH91684.1 | 16753 | 22110 | 308 | | | | |
| 1362 | 22111 | 483 | KAH96190.1 | 16755 | 22112 | 202 | KAH96191.1 | 16756 | 22113 | 308 | | | | |
| 1363 | 22114 | 483 | KAH96015.1 | 16758 | 22115 | 202 | KAH96016.1 | 16759 | 22116 | 308 | | | | |
| 1364 | 22117 | 483 | KAI00776.1 | 16761 | 22118 | 202 | KAI00777.1 | 16762 | 22119 | 308 | | | | |
| 1365 | 22120 | 483 | KAI04124.1 | 16764 | 22121 | 202 | KAI04125.1 | 16765 | 22122 | 308 | | | | |
| 1366 | 22123 | 483 | KAI02995.1 | 16767 | 22124 | 202 | KAI02996.1 | 16768 | 22125 | 308 | | | | |
| 1367 | 22126 | 483 | KAI08746.1 | 16770 | 22127 | 202 | KAI08747.1 | 16771 | 22128 | 308 | | | | |
| 1368 | 22129 | 483 | KAI10608.1 | 16773 | 22130 | 202 | KAI10609.1 | 16774 | 22131 | 308 | | | | |
| 1369 | 22132 | 483 | KAI13048.1 | 16776 | 22133 | 202 | KAI13049.1 | 16777 | 22134 | 308 | | | | |
| 1370 | 22135 | 483 | KAI15460.1 | 16779 | 22136 | 202 | KAI15461.1 | 16780 | 22137 | 308 | | | | |
| 1371 | 22138 | 375 | KAI16286.1 | 16782 | 22139 | 202 | KAI16287.1 | 16783 | 22140 | 308 | | | | |
| 1372 | 22141 | 483 | KAI19039.1 | 16785 | 22142 | 202 | KAI19040.1 | 16786 | 22143 | 308 | | | | |
| 1373 | 22144 | 483 | KAI22532.1 | 16788 | 22145 | 202 | KAI22533.1 | 16789 | 22146 | 308 | | | | |
| 1374 | 22147 | 375 | KAI26856.1 | 16791 | 22148 | 202 | KAI26857.1 | 16792 | 22149 | 308 | | | | |
| 1375 | 22150 | 375 | KAI29883.1 | 16794 | 22151 | 202 | KAI29884.1 | 16795 | 22152 | 308 | | | | |
| 1376 | 22153 | 483 | KAI31278.1 | 16797 | 22154 | 202 | KAI31279.1 | 16798 | 22155 | 308 | | | | |
| 1377 | 22156 | 483 | KAI34384.1 | 16800 | 22157 | 202 | KAI34385.1 | 16801 | 22158 | 308 | | | | |
| 1378 | 22159 | 483 | KAI37684.1 | 16803 | 22160 | 202 | KAI37685.1 | 16804 | 22161 | 308 | | | | |
| 1379 | 22162 | 483 | KAI38931.1 | 16806 | 22163 | 202 | KAI38932.1 | 16807 | 22164 | 308 | | | | |
| 1380 | 22165 | 483 | KAI42592.1 | 16809 | 22166 | 202 | KAI42593.1 | 16810 | 22167 | 308 | | | | |
| 1381 | 22168 | 483 | KAI44806.1 | 16812 | 22169 | 202 | KAI44807.1 | 16813 | 22170 | 308 | | | | |
| 1382 | 22171 | 483 | KAI46180.1 | 16815 | 22172 | 202 | KAI46181.1 | 16816 | 22173 | 308 | | | | |
| 1383 | 22174 | 483 | KAI50312.1 | 16818 | 22175 | 202 | KAI50313.1 | 16819 | 22176 | 308 | | | | |
| 1384 | 22177 | 483 | KAI52599.1 | 16821 | 22178 | 202 | KAI52600.1 | 16822 | 22179 | 308 | | | | |
| 1385 | 22180 | 483 | KAI51893.1 | 16824 | 22181 | 202 | KAI51894.1 | 16825 | 22182 | 308 | | | | |
| 1386 | 22183 | 483 | KAI58510.1 | 16827 | 22184 | 202 | KAI58511.1 | 16828 | 22185 | 308 | | | | |
| 1387 | 22186 | 483 | KAI59068.1 | 16830 | 22187 | 202 | KAI59069.1 | 16831 | 22188 | 308 | | | | |
| 1388 | 22189 | 375 | KAI62092.1 | 16833 | 22190 | 202 | KAI62093.1 | 16834 | 22191 | 308 | | | | |
| 1389 | 22192 | 483 | KAI65036.1 | 16836 | 22193 | 202 | KAI65037.1 | 16837 | 22194 | 308 | | | | |
| 1390 | 22195 | 483 | KAI67114.1 | 16839 | 22196 | 202 | KAI67115.1 | 16840 | 22197 | 72 | | | | |
| 1391 | 22198 | 483 | KAI71115.1 | 16842 | 22199 | 202 | KAI71116.1 | 16843 | 22200 | 72 | | | | |
| 1392 | 22201 | 483 | KAI72296.1 | 16845 | 22202 | 202 | KAI72297.1 | 16846 | 22203 | 308 | | | | |
| 1393 | 22204 | 483 | KAI76026.1 | 16848 | 22205 | 202 | KAI76027.1 | 16849 | 22206 | 308 | | | | |
| 1394 | 22207 | 483 | KAI77489.1 | 16851 | 22208 | 202 | KAI77490.1 | 16852 | 22209 | 308 | | | | |
| 1395 | 22210 | 483 | KAI79405.1 | 16854 | 22211 | 202 | KAI79406.1 | 16855 | 22212 | 308 | | | | |
| 1396 | 22213 | 483 | KAI83419.1 | 16857 | 22214 | 202 | KAI83420.1 | 16858 | 22215 | 308 | | | | |
| 1397 | 22216 | 483 | KAF82850.1 | 16860 | 22217 | 202 | KAF82851.1 | 16861 | 22218 | 308 | | | | |
| 1398 | 22219 | 487 | KES25285.1 | 16863 | 22220 | 167 | KES25283.1 | 16864 | 22221 | 183 | | | | |
| 1399 | 22222 | 487 | KKB51169.1 | 16866 | 22223 | 160 | KKB51171.1 | 16867 | 22224 | 186 | | | | |
| 1400 | 22225 | 481 | KKB46311.1 | 16869 | 22226 | 163 | KKB46308.1 | 16870 | 22227 | 181 | | | | |
| 1401 | 22228 | 478 | KME80491.1 | 16872 | 22229 | 203 | | | | | | | | |
| 1402 | 22230 | 484 | KMJ04348.1 | 16874 | 22231 | 163 | | | | | | | | |
| 1403 | 22232 | 480 | KSU86124.1 | 16876 | 22233 | 202 | | | | | | | | |
| 1404 | 22234 | 484 | KUR80454.1 | 16878 | 22235 | 165 | KUR80452.1 | 16879 | 22236 | 184 | KUR80451.1 | 16880 | 22237 | 259 |
| 1405 | 22238 | 476 | KXT60078.1 | 16882 | 22239 | 207 | | | | | | | | |
| 1406 | 22240 | 477 | KJP12012.1 | 16884 | 22241 | 146 | KJP12010.1 | 16885 | 22242 | 161 | | | | |
| 1407 | 22243 | 486 | KKO42007.1 | 16887 | 22244 | 202 | | | | | | | | |
| 1408 | 22245 | 480 | KMQ31910.1 | 16889 | 22246 | 202 | | | | | | | | |
| 1409 | 22247 | 480 | KMQ31455.1 | 16891 | 22248 | 202 | | | | | | | | |
| 1410 | 22249 | 480 | KMQ31338.1 | 16893 | 22250 | 202 | | | | | | | | |
| 1411 | 22251 | 477 | KMQ07577.1 | 16895 | 22252 | 226 | | | | | | | | |
| 1412 | 22253 | 488 | KJU85604.1 | 16897 | 22254 | 162 | | | | | | | | |
| 1413 | 22255 | 475 | KJS18753.1 | 16899 | 22256 | 160 | KJS18754.1 | 16900 | 22257 | 228 | | | | |
| 1414 | 22258 | 478 | KLD44467.1 | 16902 | 22259 | 203 | | | | | | | | |
| 1415 | 22260 | 481 | KKZ08482.1 | 16904 | 22261 | 200 | KKZ08481.1 | 16905 | 22262 | 191 | | | | |
| 1416 | 22263 | 479 | KKX43504.1 | 16907 | 22264 | 209 | | | | | | | | |
| 1417 | 22265 | 478 | KKJ66888.1 | 16909 | 22266 | 161 | KKJ66890.1 | 16910 | 22267 | 283 | | | | |
| 1418 | 22268 | 480 | KKR96939.1 | 16912 | 22269 | 168 | KKR96941.1 | 16913 | 22270 | 393 | | | | |
| 1419 | 22271 | 484 | KKZ15777.1 | 16915 | 22272 | 163 | | | | | | | | |
| 1420 | 22273 | 586 | KLA35046.1 | 16917 | 22274 | 325 | | | | | | | | |
| 1421 | 22275 | 477 | KLA36291.1 | 16919 | 22276 | 226 | | | | | | | | |
| 1422 | 22277 | 481 | KLF79288.1 | 16921 | 22278 | 155 | | | | | | | | |
| 1423 | 22279 | 484 | KLE38478.1 | 16923 | 22280 | 163 | | | | | | | | |
| 1424 | 22281 | 481 | KMN72582.1 | 16925 | 22282 | 202 | | | | | | | | |
| 1425 | 22283 | 481 | KMN78964.1 | 16927 | 22284 | 155 | KMN78968.1 | 16928 | 22285 | 155 | KMN78965.1 | 16929 | 22286 | 190 |
| 1426 | 22287 | 477 | KOQ86135.1 | 16931 | 22288 | 146 | KOQ86133.1 | 16932 | 22289 | 161 | | | | |
| 1427 | 22290 | 479 | KOQ96627.1 | 16934 | 22291 | 205 | KOQ96628.1 | 16935 | 22292 | 165 | | | | |
| 1428 | 22293 | 487 | KXU94745.1 | 16937 | 22294 | 167 | KXU94712.1 | 16938 | 22295 | 183 | | | | |
| 1429 | 22296 | 484 | KMU49984.1 | 16940 | 22297 | 163 | | | | | | | | |
| 1430 | 22298 | 269 | KMZ12178.1 | 16942 | 22299 | 103 | KMZ12179.1 | 16943 | 22300 | 174 | | | | |
| 1431 | 22301 | 477 | KMY27374.1 | 16945 | 22302 | 155 | KMY27378.1 | 16946 | 22303 | 147 | KMY27376.1 | 16947 | 22304 | 165 |
| 1432 | 22305 | 481 | KOF39298.1 | 16949 | 22306 | 200 | KOF39299.1 | 16950 | 22307 | 191 | | | | |
| 1433 | 22308 | 492 | KPH74234.1 | 16952 | 22309 | 161 | KPH74237.1 | 16953 | 22310 | 175 | | | | |
| 1434 | 22311 | 487 | KPG99047.1 | 16955 | 22312 | 167 | KPG99049.1 | 16956 | 22313 | 183 | | | | |
| 1435 | 22314 | 486 | KUG12362.1 | 16958 | 22315 | 163 | KUG12363.1 | 16959 | 22316 | 235 | KUG12375.1 | 16960 | 22317 | 178 |
| 1436 | 22318 | 477 | KPS74517.1 | 16962 | 22319 | 200 | KPS74518.1 | 16963 | 22320 | 146 | KPS74516.1 | 16964 | 22321 | 395 |
| 1437 | 22322 | 477 | KPS95937.1 | 16966 | 22323 | 146 | KPS95938.1 | 16967 | 22324 | 200 | KPS95939.1 | 16968 | 22325 | 395 |
| 1438 | 22326 | 487 | KPF70567.1 | 16970 | 22327 | 165 | | | | | | | | |
| 1439 | 22328 | 477 | KQB82199.1 | 16972 | 22329 | 202 | | | | | | | | |
| 1440 | 22330 | 474 | KQL33556.1 | 16974 | 22331 | 222 | | | | | | | | |
| 1441 | 22332 | 477 | KPY89191.1 | 16976 | 22333 | 147 | KPY89133.1 | 16977 | 22334 | 167 | KPY89165.1 | 16978 | 22335 | 276 |
| 1442 | 22336 | 482 | CDT62295.1 | 16980 | 22337 | 166 | CDT62262.1 | 16981 | 22338 | 228 | | | | |
| 1443 | 22339 | 482 | CDT49130.1 | 16983 | 22340 | 166 | CDT49099.1 | 16984 | 22341 | 228 | | | | |
| 1444 | 22342 | 475 | KTC51735.1 | 16986 | 22343 | 160 | | | | | | | | |
| 1445 | 22344 | 475 | KTB70324.1 | 16988 | 22345 | 160 | | | | | | | | |
| 1446 | 22346 | 494 | KTB90115.1 | 16990 | 22347 | 312 | | | | | | | | |
| 1447 | 22348 | 388 | KTC03052.1 | 16992 | 22349 | 231 | | | | | | | | |
| 1448 | 22350 | 474 | KRF62305.1 | 16994 | 22351 | 200 | | | | | | | | |
| 1449 | 22352 | 474 | KSF14310.1 | 16996 | 22353 | 163 | | | | | | | | |
| 1450 | 22354 | 482 | KSG00434.1 | 16998 | 22355 | 158 | KSG00432.1 | 16999 | 22356 | 179 | KSG00433.1 | 17000 | 22357 | 183 |
| 1451 | 22359 | 482 | KSO26595.1 | 17003 | 22360 | 158 | KSO26596.1 | 17004 | 22361 | 183 | KSO26597.1 | 17005 | 22362 | 179 |
| 1452 | 22364 | 482 | KSQ24405.1 | 17008 | 22365 | 158 | KSQ24406.1 | 17009 | 22366 | 183 | KSQ24407.1 | 17010 | 22367 | 179 |
| 1453 | 22369 | 487 | KSQ18858.1 | 17013 | 22370 | 167 | KSQ18860.1 | 17014 | 22371 | 183 | | | | |
| 1454 | 22372 | 501 | KQV52822.1 | 17016 | 22373 | 158 | | | | | | | | |
| 1455 | 22374 | 596 | KQW19001.1 | 17018 | 22375 | 370 | | | | | | | | |
| 1456 | 22376 | 480 | KQN02370.1 | 17020 | 22377 | 158 | | | | | | | | |
| 1457 | 22378 | 476 | KQS95551.1 | 17022 | 22379 | 205 | KQS95550.1 | 17023 | 22380 | 174 | | | | |
| 1458 | 22381 | 334 | KRE80989.1 | 17025 | 22382 | 318 | | | | | | | | |
| 1459 | 22383 | 240 | CEA11437.1 | 17027 | 22384 | 156 | | | | | | | | |
| 1460 | 22385 | 240 | CEA10433.1 | 17029 | 22386 | 156 | | | | | | | | |
| 1461 | 22387 | 307 | CUU52510.1 | 17031 | 22388 | 165 | CUU52511.1 | 17032 | 22389 | 214 | | | | |
| 1462 | 22390 | 482 | KSX76370.1 | 17034 | 22391 | 166 | | | | | | | | |
| 1463 | 22392 | 478 | KWY00932.1 | 17036 | 22393 | 161 | KWY00934.1 | 17037 | 22394 | 283 | | | | |
| 1464 | 22395 | 478 | KWY16708.1 | 17039 | 22396 | 161 | KWY16706.1 | 17040 | 22397 | 283 | | | | |
| 1465 | 22398 | 478 | KWY36997.1 | 17042 | 22399 | 161 | KWY36995.1 | 17043 | 22400 | 283 | | | | |
| 1466 | 22401 | 329 | KWY51063.1 | 17045 | 22402 | 161 | | | | | | | | |
| 1467 | 22403 | 314 | KWY60249.1 | 17047 | 22404 | 161 | | | | | | | | |
| 1468 | 22405 | 478 | KWY71635.1 | 17049 | 22406 | 161 | | | | | | | | |
| 1469 | 22407 | 478 | KWY79779.1 | 17051 | 22408 | 161 | KWY79777.1 | 17052 | 22409 | 283 | | | | |
| 1470 | 22410 | 478 | KWY89824.1 | 17054 | 22411 | 114 | | | | | | | | |
| 1471 | 22412 | 478 | KWY92723.1 | 17056 | 22413 | 161 | KWY92721.1 | 17057 | 22414 | 283 | | | | |
| 1472 | 22415 | 478 | KWY92991.1 | 17059 | 22416 | 161 | KWY92993.1 | 17060 | 22417 | 283 | | | | |
| 1473 | 22418 | 478 | KWY96006.1 | 17062 | 22419 | 161 | KWY96008.1 | 17063 | 22420 | 283 | | | | |
| 1474 | 22421 | 478 | KWZ06303.1 | 17065 | 22422 | 161 | KWZ06305.1 | 17066 | 22423 | 283 | | | | |
| 1475 | 22424 | 489 | KST54507.1 | 17068 | 22425 | 202 | KST54506.1 | 17069 | 22426 | 344 | | | | |
| 1476 | 22427 | 477 | KTD46364.1 | 17071 | 22428 | 163 | | | | | | | | |
| 1477 | 22429 | 479 | KUO54663.1 | 17073 | 22430 | 155 | | | | | | | | |
| 1478 | 22431 | 500 | KXY14128.1 | 17075 | 22432 | 206 | | | | | | | | |
| 1479 | 22433 | 126 | KXQ35633.1 | 17077 | 22434 | 155 | | | | | | | | |
| 1480 | 22435 | 126 | KXG66577.1 | 17079 | 22436 | 155 | | | | | | | | |
| 1481 | 22437 | 476 | KVM18169.1 | 17081 | 22438 | 160 | KVM18174.1 | 17082 | 22439 | 155 | KVM18170.1 | 17083 | 22440 | 197 |
| 1482 | 22441 | 476 | KVM22188.1 | 17085 | 22442 | 155 | KVM22193.1 | 17086 | 22443 | 160 | KVM22192.1 | 17087 | 22444 | 197 |
| 1483 | 22445 | 476 | KVM54442.1 | 17089 | 22446 | 160 | KVM54448.1 | 17090 | 22447 | 155 | KVM54443.1 | 17091 | 22448 | 197 |
| 1484 | 22449 | 476 | KVO16516.1 | 17093 | 22450 | 146 | | | | | | | | |
| 1485 | 22451 | 536 | KVR44247.1 | 17095 | 22452 | 315 | | | | | | | | |
| 1486 | 22453 | 484 | KVR99615.1 | 17097 | 22454 | 189 | KVR99752.1 | 17098 | 22455 | 129 | | | | |
| 1487 | 22456 | 477 | KVS07628.1 | 17100 | 22457 | 203 | | | | | | | | |
| 1488 | 22458 | 484 | KVU94134.1 | 17102 | 22459 | 189 | KVU94209.1 | 17103 | 22460 | 129 | | | | |
| 1489 | 22461 | 476 | KVX54695.1 | 17105 | 22462 | 160 | | | | | | | | |
| 1490 | 22463 | 493 | KWA77748.1 | 17107 | 22464 | 189 | KWA77756.1 | 17108 | 22465 | 129 | | | | |
| 1491 | 22466 | 493 | KWB32212.1 | 17110 | 22467 | 189 | KWB32219.1 | 17111 | 22468 | 129 | | | | |
| 1492 | 22469 | 536 | KWB80881.1 | 17113 | 22470 | 315 | | | | | | | | |
| 1493 | 22471 | 481 | KTI37618.1 | 17115 | 22472 | 155 | KTI37617.1 | 17116 | 22473 | 280 | | | | |
| 1494 | 22474 | 529 | KTJ90667.1 | 17118 | 22475 | 323 | | | | | | | | |
| 1495 | 22476 | 481 | KXM51395.1 | 17120 | 22477 | 155 | | | | | | | | |
| 1496 | 22478 | 481 | KXM54362.1 | 17122 | 22479 | 155 | | | | | | | | |
| 1497 | 22480 | 481 | KXM99994.1 | 17124 | 22481 | 155 | | | | | | | | |
| 1498 | 22482 | 478 | KXH57775.1 | 17126 | 22483 | 161 | KXH57777.1 | 17127 | 22484 | 283 | | | | |
| 1499 | 22485 | 482 | KUS35847.1 | 17129 | 22486 | 166 | | | | | | | | |
| 1500 | 22487 | 482 | KUT10060.1 | 17131 | 22488 | 166 | | | | | | | | |
| 1501 | 22489 | 481 | KUT23042.1 | 17133 | 22490 | 155 | | | | | | | | |
| 1502 | 22491 | 482 | KUT23266.1 | 17135 | 22492 | 166 | | | | | | | | |
| 1503 | 22493 | 482 | KUT30719.1 | 17137 | 22494 | 166 | | | | | | | | |
| 1504 | 22495 | 481 | KUT68248.1 | 17139 | 22496 | 155 | | | | | | | | |
| 1505 | 22497 | 481 | KUU91588.1 | 17141 | 22498 | 155 | | | | | | | | |
| 1506 | 22499 | 482 | KUX27114.1 | 17143 | 22500 | 166 | | | | | | | | |
| 1507 | 22501 | 481 | KUQ77104.1 | 17145 | 22502 | 155 | | | | | | | | |
| 1508 | 22503 | 481 | KUQ90477.1 | 17147 | 22504 | 155 | | | | | | | | |
| 1509 | 22505 | 477 | KVX01472.1 | 17149 | 22506 | 147 | | | | | | | | |
| 1510 | 22507 | 481 | KVK21769.1 | 17151 | 22508 | 155 | | | | | | | | |
| 1511 | 22509 | 103 | KYQ15614.1 | 17153 | 22510 | 155 | KYQ15615.1 | 17154 | 22511 | 192 | | | | |
| 1512 | 22512 | 103 | KYQ10110.1 | 17156 | 22513 | 155 | KYQ10109.1 | 17157 | 22514 | 192 | | | | |
| 1513 | 22515 | 103 | KYQ13515.1 | 17159 | 22516 | 155 | KYQ13514.1 | 17160 | 22517 | 192 | | | | |
| 1514 | 22518 | 103 | KYQ20475.1 | 17162 | 22519 | 155 | KYQ20476.1 | 17163 | 22520 | 192 | | | | |
| 1515 | 22521 | 487 | KXA74274.1 | 17165 | 22522 | 239 | | | | | | | | |
| 1516 | 22523 | 1197 | AAB91634.1 | 17167 | 22524 | 640 | | | | | | | | |
| 1517 | 22525 | 278 | ABG41253.1 | 17169 | 22526 | 189 | | | | | | | | |
| 1518 | 22527 | 282 | ACM32748.1 | 17171 | 22528 | 187 | | | | | | | | |
| 1519 | 22529 | 296 | ACR72775.1 | 17173 | 22530 | 184 | | | | | | | | |
| 1520 | 22531 | 297 | ACR74434.1 | 17175 | 22532 | 188 | | | | | | | | |
| 1521 | 22533 | 283 | ADY24841.1 | 17177 | 22534 | 189 | | | | | | | | |
| 1522 | 22535 | 297 | AFJ62118.1 | 17179 | 22536 | 156 | | | | | | | | |
| 1523 | 22537 | 278 | AHC83881.1 | 17181 | 22538 | 187 | | | | | | | | |
| 1524 | 22539 | 278 | AHC89252.1 | 17183 | 22540 | 187 | | | | | | | | |
| 1525 | 22541 | 285 | AHJ13795.1 | 17185 | 22542 | 187 | | | | | | | | |
| 1526 | 22543 | 278 | AIL63103.1 | 17187 | 22544 | 187 | | | | | | | | |
| 1527 | 22545 | 278 | AIX52586.1 | 17189 | 22546 | 184 | | | | | | | | |
| 1528 | 22547 | 278 | AIX76374.1 | 17191 | 22548 | 184 | | | | | | | | |
| 1529 | 22549 | 275 | AJE78820.1 | 17193 | 22550 | 190 | | | | | | | | |
| 1530 | 22551 | 278 | AKL33256.1 | 17195 | 22552 | 184 | | | | | | | | |
| 1531 | 22553 | 92 | BAB73899.1 | 17197 | 22554 | 186 | | | | | | | | |
| 1532 | 22555 | 282 | CAD16318.1 | 17199 | 22556 | 187 | | | | | | | | |
| 1533 | 22557 | 285 | CBK39911.1 | 17201 | 22558 | 189 | | | | | | | | |
| 1534 | 22559 | 297 | CCG49954.1 | 17203 | 22560 | 190 | | | | | | | | |
| 1535 | 22561 | 156 | CDE97487.1 | 17205 | 22562 | 186 | | | | | | | | |
| 1536 | 22563 | 307 | CDG89551.1 | 17207 | 22564 | 187 | | | | | | | | |
| 1537 | 22565 | 277 | CDG92167.1 | 17209 | 22566 | 187 | | | | | | | | |
| 1538 | 22567 | 278 | CDZ76666.1 | 17211 | 22568 | 187 | | | | | | | | |
| 1539 | 22569 | 278 | CEE12404.1 | 17213 | 22570 | 187 | | | | | | | | |
| 1540 | 22571 | 282 | CUJ73678.1 | 17215 | 22572 | 187 | | | | | | | | |
| 1541 | 22573 | 278 | CVH06653.1 | 17217 | 22574 | 187 | | | | | | | | |
| 1542 | 22575 | 278 | CVH45994.1 | 17219 | 22576 | 185 | | | | | | | | |
| 1543 | 22577 | 278 | CZP78194.1 | 17221 | 22578 | 187 | | | | | | | | |
| 1544 | 22579 | 278 | CZQ07179.1 | 17223 | 22580 | 187 | | | | | | | | |
| 1545 | 22581 | 282 | EAZ59086.1 | 17225 | 22582 | 187 | | | | | | | | |
| 1546 | 22583 | 286 | EEG71311.1 | 17227 | 22584 | 188 | | | | | | | | |
| 1547 | 22585 | 297 | EEU99050.1 | 17229 | 22586 | 188 | | | | | | | | |
| 1548 | 22587 | 278 | EHL32739.1 | 17231 | 22588 | 187 | | | | | | | | |
| 1549 | 22589 | 278 | EHT09459.1 | 17233 | 22590 | 184 | | | | | | | | |
| 1550 | 22591 | 187 | EJO27434.1 | 17235 | 22592 | 99 | | | | | | | | |
| 1551 | 22593 | 278 | ENO98789.1 | 17237 | 22594 | 187 | | | | | | | | |
| 1552 | 22595 | 282 | EPD37370.1 | 17239 | 22596 | 187 | | | | | | | | |
| 1553 | 22597 | 283 | ERI11821.1 | 17241 | 22598 | 189 | | | | | | | | |
| 1554 | 22599 | 282 | ERV92400.1 | 17243 | 22600 | 187 | | | | | | | | |
| 1555 | 22601 | 278 | ESF54844.1 | 17245 | 22602 | 184 | | | | | | | | |
| 1556 | 22603 | 278 | ESM46332.1 | 17247 | 22604 | 186 | | | | | | | | |
| 1557 | 22605 | 278 | EUL65866.1 | 17249 | 22606 | 186 | | | | | | | | |
| 1558 | 22607 | 278 | EUL69872.1 | 17251 | 22608 | 186 | | | | | | | | |
| 1559 | 22609 | 278 | EWC39937.1 | 17253 | 22610 | 85 | | | | | | | | |
| 1560 | 22611 | 278 | EXS68250.1 | 17255 | 22612 | 187 | | | | | | | | |
| 1561 | 22613 | 283 | KEP24868.1 | 17257 | 22614 | 190 | | | | | | | | |
| 1562 | 22615 | 280 | KFC21767.1 | 17259 | 22616 | 187 | | | | | | | | |
| 1563 | 22617 | 275 | KFI14245.1 | 17261 | 22618 | 190 | | | | | | | | |
| 1564 | 22619 | 282 | KFI74924.1 | 17263 | 22620 | 196 | | | | | | | | |
| 1565 | 22621 | 278 | KHJ15950.1 | 17265 | 22622 | 184 | | | | | | | | |
| 1566 | 22623 | 278 | KIZ39819.1 | 17267 | 22624 | 184 | | | | | | | | |
| 1567 | 22625 | 278 | KJB99791.1 | 17269 | 22626 | 184 | | | | | | | | |
| 1568 | 22627 | 278 | KJN38559.1 | 17271 | 22628 | 184 | | | | | | | | |
| 1569 | 22629 | 277 | KJV39509.1 | 17273 | 22630 | 185 | | | | | | | | |
| 1570 | 22631 | 276 | KML18442.1 | 17275 | 22632 | 190 | | | | | | | | |
| 1571 | 22633 | 276 | KML61951.1 | 17277 | 22634 | 190 | | | | | | | | |
| 1572 | 22635 | 276 | KMN31256.1 | 17279 | 22636 | 190 | | | | | | | | |
| 1573 | 22637 | 276 | KMN70227.1 | 17281 | 22638 | 190 | | | | | | | | |
| 1574 | 22639 | 282 | KOC82291.1 | 17283 | 22640 | 190 | | | | | | | | |
| 1575 | 22641 | 278 | KOY23386.1 | 17285 | 22642 | 187 | | | | | | | | |
| 1576 | 22643 | 291 | KQQ38325.1 | 17287 | 22644 | 185 | | | | | | | | |
| 1577 | 22645 | 282 | KRG37250.1 | 17289 | 22646 | 187 | | | | | | | | |
| 1578 | 22647 | 282 | KRG58644.1 | 17291 | 22648 | 187 | | | | | | | | |
| 1579 | 22649 | 278 | KSC65529.1 | 17293 | 22650 | 187 | | | | | | | | |
| 1580 | 22651 | 282 | KSQ38236.1 | 17295 | 22652 | 187 | | | | | | | | |
| 1581 | 22653 | 297 | KSV97270.1 | 17297 | 22654 | 190 | | | | | | | | |
| 1582 | 22655 | 297 | KSV97838.1 | 17299 | 22656 | 190 | | | | | | | | |
| 1583 | 22657 | 297 | KTF62016.1 | 17301 | 22658 | 190 | | | | | | | | |
| 1584 | 22659 | 280 | KUJ59694.1 | 17303 | 22660 | 188 | | | | | | | | |
| 1585 | 22661 | 292 | KUR72679.1 | 17305 | 22662 | 186 | | | | | | | | |
| 1586 | 22663 | 278 | KVL39771.1 | 17307 | 22664 | 187 | | | | | | | | |
| 1587 | 22665 | 285 | KYG88424.1 | 17309 | 22666 | 187 | | | | | | | | |
| 1588 | 22667 | 278 | ABG69916.1 | 17311 | 22668 | 287 | | | | | | | | |
| 1589 | 22669 | 278 | CCQ05513.1 | 17313 | 22670 | 287 | | | | | | | | |
| 1590 | 22671 | 278 | EZJ21711.1 | 17315 | 22672 | 307 | | | | | | | | |
| 1591 | 22673 | 278 | EZJ42019.1 | 17317 | 22674 | 307 | | | | | | | | |
| 1592 | 22675 | 278 | EZJ61190.1 | 17319 | 22676 | 307 | | | | | | | | |
| 1593 | 22677 | 278 | KEJ12466.1 | 17321 | 22678 | 287 | | | | | | | | |
| 1594 | 22679 | 278 | KEJ13625.1 | 17323 | 22680 | 287 | | | | | | | | |
| 1595 | 22681 | 278 | KEJ33278.1 | 17325 | 22682 | 287 | | | | | | | | |
| 1596 | 22683 | 278 | KEN34069.1 | 17327 | 22684 | 287 | | | | | | | | |
| 1597 | 22685 | 278 | KEN41711.1 | 17329 | 22686 | 287 | | | | | | | | |
| 1598 | 22687 | 278 | KEN74068.1 | 17331 | 22688 | 287 | | | | | | | | |
| 1599 | 22689 | 278 | KKA59532.1 | 17333 | 22690 | 287 | | | | | | | | |
| 1600 | 22691 | 226 | ABO13065.2 | 17335 | 22692 | 169 | | | | | | | | |
| 1601 | 22693 | 278 | AGN81862.1 | 17337 | 22694 | 169 | | | | | | | | |
| 1602 | 22695 | 292 | AKC78199.1 | 17339 | 22696 | 166 | | | | | | | | |
| 1603 | 22697 | 270 | AMP74180.1 | 17341 | 22698 | 185 | | | | | | | | |
| 1604 | 22699 | 285 | ENW63935.1 | 17343 | 22700 | 169 | | | | | | | | |
| 1605 | 22701 | 292 | KJZ35757.1 | 17345 | 22702 | 166 | | | | | | | | |
| 1606 | 22703 | 292 | KSM97146.1 | 17347 | 22704 | 166 | | | | | | | | |
| 1607 | 22705 | 285 | KWX65127.1 | 17349 | 22706 | 169 | | | | | | | | |
| 1608 | 22707 | 278 | KJX33868.1 | 17351 | 22708 | 169 | | | | | | | | |
| 1609 | 22709 | 278 | EHW83929.1 | 17353 | 22710 | 169 | | | | | | | | |
| 1610 | 22711 | 278 | EHX90141.1 | 17355 | 22712 | 169 | | | | | | | | |
| 1611 | 22713 | 278 | EOW20699.1 | 17357 | 22714 | 169 | | | | | | | | |
| 1612 | 22715 | 278 | EQT99521.1 | 17359 | 22716 | 169 | | | | | | | | |
| 1613 | 22717 | 278 | ESE28057.1 | 17361 | 22718 | 148 | | | | | | | | |
| 1614 | 22719 | 278 | KDA72452.1 | 17363 | 22720 | 169 | | | | | | | | |
| 1615 | 22721 | 278 | KEJ24821.1 | 17365 | 22722 | 148 | | | | | | | | |
| 1616 | 22723 | 278 | KEJ23492.1 | 17367 | 22724 | 148 | | | | | | | | |
| 1617 | 22725 | 278 | KEL24739.1 | 17369 | 22726 | 169 | | | | | | | | |
| 1618 | 22727 | 278 | KEL41025.1 | 17371 | 22728 | 169 | | | | | | | | |
| 1619 | 22729 | 278 | KEL61645.1 | 17373 | 22730 | 169 | | | | | | | | |
| 1620 | 22731 | 278 | KIG27436.1 | 17375 | 22732 | 169 | | | | | | | | |
| 1621 | 22733 | 278 | CTV70042.1 | 17377 | 22734 | 169 | | | | | | | | |
| 1622 | 22735 | 278 | ESL95858.1 | 17379 | 22736 | 188 | | | | | | | | |
| 1623 | 22737 | 278 | EZR09257.1 | 17381 | 22738 | 188 | | | | | | | | |
| 1624 | 22739 | 278 | KMI15681.1 | 17383 | 22740 | 188 | | | | | | | | |
| 1625 | 22741 | 278 | BAO34670.1 | 17385 | 22742 | 169 | | | | | | | | |
| 1626 | 22743 | 278 | CNG20796.1 | 17387 | 22744 | 169 | | | | | | | | |
| 1627 | 22745 | 278 | CNJ07156.1 | 17389 | 22746 | 169 | | | | | | | | |
| 1628 | 22747 | 286 | KUO23348.1 | 17391 | 22748 | 183 | | | | | | | | |
| 1629 | 22749 | 277 | AKA50671.1 | 17393 | 22750 | 173 | | | | | | | | |
| 1630 | 22751 | 276 | EKN28635.1 | 17395 | 22752 | 170 | | | | | | | | |
| 1631 | 22753 | 276 | EEB34164.1 | 17397 | 22754 | 152 | | | | | | | | |
| 1632 | 22755 | 370 | ABE61673.1 | 17399 | 22756 | 183 | | | | | | | | |
| 1633 | 22757 | 286 | EKS39931.1 | 17401 | 22758 | 183 | | | | | | | | |
| 1634 | 22759 | 285 | KGB50926.1 | 17403 | 22760 | 166 | | | | | | | | |
| 1635 | 22761 | 290 | EFL96229.1 | 17405 | 22762 | 158 | | | | | | | | |
| 1636 | 22763 | 277 | EGF16081.1 | 17407 | 22764 | 166 | | | | | | | | |
| 1637 | 22765 | 275 | CYU22341.1 | 17409 | 22766 | 169 | | | | | | | | |
| 1638 | 22767 | 275 | CYX44050.1 | 17411 | 22768 | 169 | | | | | | | | |
| 1639 | 22769 | 275 | EMB57080.1 | 17413 | 22770 | 166 | | | | | | | | |
| 1640 | 22771 | 275 | EMC13031.1 | 17415 | 22772 | 166 | | | | | | | | |
| 1641 | 22773 | 275 | AMF85253.1 | 17417 | 22774 | 166 | | | | | | | | |
| 1642 | 22775 | 275 | EFV96551.1 | 17419 | 22776 | 166 | | | | | | | | |
| 1643 | 22777 | 212 | EGS28537.1 | 17421 | 22778 | 166 | | | | | | | | |
| 1644 | 22779 | 275 | EPT39846.1 | 17423 | 22780 | 166 | | | | | | | | |
| 1645 | 22781 | 212 | EPV87466.1 | 17425 | 22782 | 166 | | | | | | | | |
| 1646 | 22783 | 78 | EPV95020.1 | 17427 | 22784 | 166 | | | | | | | | |
| 1647 | 22785 | 275 | KLL29855.1 | 17429 | 22786 | 166 | | | | | | | | |
| 1648 | 22787 | 275 | KLL34441.1 | 17431 | 22788 | 166 | | | | | | | | |
| 1649 | 22789 | 212 | KLL34968.1 | 17433 | 22790 | 166 | | | | | | | | |
| 1650 | 22791 | 275 | KKF49830.1 | 17435 | 22792 | 166 | | | | | | | | |
| 1651 | 22793 | 302 | EGG58908.1 | 17437 | 22794 | 186 | | | | | | | | |
| 1652 | 22795 | 284 | EOL13669.1 | 17439 | 22796 | 186 | | | | | | | | |
| 1653 | 22797 | 284 | EOK19567.1 | 17441 | 22798 | 186 | | | | | | | | |
| 1654 | 22799 | 284 | EOJ40183.1 | 17443 | 22800 | 186 | | | | | | | | |
| 1655 | 22801 | 284 | EOJ44766.1 | 17445 | 22802 | 186 | | | | | | | | |
| 1656 | 22803 | 291 | EPI04162.1 | 17447 | 22804 | 186 | | | | | | | | |
| 1657 | 22805 | 284 | ETT98568.1 | 17449 | 22806 | 186 | | | | | | | | |
| 1658 | 22807 | 284 | ETU01532.1 | 17451 | 22808 | 186 | | | | | | | | |
| 1659 | 22809 | 284 | ETU06237.1 | 17453 | 22810 | 186 | | | | | | | | |
| 1660 | 22811 | 284 | ETU10203.1 | 17455 | 22812 | 186 | | | | | | | | |
| 1661 | 22813 | 284 | ETU16614.1 | 17457 | 22814 | 186 | | | | | | | | |
| 1662 | 22815 | 284 | ETU21339.1 | 17459 | 22816 | 186 | | | | | | | | |
| 1663 | 22817 | 284 | ETU24457.1 | 17461 | 22818 | 186 | | | | | | | | |
| 1664 | 22819 | 284 | ETU31994.1 | 17463 | 22820 | 186 | | | | | | | | |
| 1665 | 22821 | 284 | ETU40538.1 | 17465 | 22822 | 186 | | | | | | | | |
| 1666 | 22823 | 284 | ETU48374.1 | 17467 | 22824 | 186 | | | | | | | | |
| 1667 | 22825 | 277 | CDG04461.1 | 17469 | 22826 | 166 | | | | | | | | |
| 1668 | 22827 | 259 | KMT21892.1 | 17471 | 22828 | 174 | | | | | | | | |
| 1669 | 22829 | 285 | AMN36764.1 | 17473 | 22830 | 202 | | | | | | | | |
| 1670 | 22831 | 278 | CEQ11380.1 | 17475 | 22832 | 200 | | | | | | | | |
| 1671 | 22833 | 275 | CDG36275.1 | 17477 | 22834 | 170 | | | | | | | | |
| 1672 | 22835 | 271 | EHG33945.1 | 17479 | 22836 | 203 | | | | | | | | |
| 1673 | 22837 | 271 | KEH95235.1 | 17481 | 22838 | 203 | | | | | | | | |
| 1674 | 22839 | 285 | ERK44587.1 | 17483 | 22840 | 164 | | | | | | | | |
| 1675 | 22841 | 285 | KRK21386.1 | 17485 | 22842 | 148 | | | | | | | | |
| 1676 | 22843 | 286 | EGE38622.1 | 17487 | 22844 | 390 | | | | | | | | |
| 1677 | 22845 | 269 | AMS04502.1 | 17489 | 22846 | 171 | | | | | | | | |
| 1678 | 22847 | 286 | EZP69578.1 | 17491 | 22848 | 181 | | | | | | | | |
| 1679 | 22849 | 271 | KJJ86349.1 | 17493 | 22850 | 231 | | | | | | | | |
| 1680 | 22851 | 280 | CAH34733.1 | 17495 | 22852 | 185 | | | | | | | | |
| 1681 | 22853 | 265 | ABN91208.1 | 17497 | 22854 | 185 | | | | | | | | |
| 1682 | 22855 | 265 | EDO90950.1 | 17499 | 22856 | 185 | | | | | | | | |
| 1683 | 22857 | 265 | EET07925.1 | 17501 | 22858 | 185 | | | | | | | | |
| 1684 | 22859 | 265 | EES25673.1 | 17503 | 22860 | 185 | | | | | | | | |
| 1685 | 22861 | 207 | EIF62859.1 | 17505 | 22862 | 185 | | | | | | | | |
| 1686 | 22863 | 207 | EIF64460.1 | 17507 | 22864 | 185 | | | | | | | | |
| 1687 | 22865 | 207 | EIF75598.1 | 17509 | 22866 | 185 | | | | | | | | |
| 1688 | 22867 | 207 | EIF80280.1 | 17511 | 22868 | 185 | | | | | | | | |
| 1689 | 22869 | 265 | AFR14681.1 | 17513 | 22870 | 177 | | | | | | | | |
| 1690 | 22871 | 263 | AIO13871.1 | 17515 | 22872 | 185 | | | | | | | | |
| 1691 | 22873 | 263 | AIO91464.1 | 17517 | 22874 | 185 | | | | | | | | |
| 1692 | 22875 | 263 | AJX61766.1 | 17519 | 22876 | 185 | | | | | | | | |
| 1693 | 22877 | 273 | KGC79013.1 | 17521 | 22878 | 185 | | | | | | | | |
| 1694 | 22879 | 263 | KGD17019.1 | 17523 | 22880 | 185 | | | | | | | | |
| 1695 | 22881 | 273 | AIS48740.1 | 17525 | 22882 | 185 | | | | | | | | |
| 1696 | 22883 | 280 | AIV89829.1 | 17527 | 22884 | 185 | | | | | | | | |
| 1697 | 22885 | 280 | AIV96226.1 | 17529 | 22886 | 185 | | | | | | | | |
| 1698 | 22887 | 263 | KGX68791.1 | 17531 | 22888 | 185 | | | | | | | | |
| 1699 | 22889 | 280 | KGY01706.1 | 17533 | 22890 | 185 | | | | | | | | |
| 1700 | 22891 | 280 | KGY04132.1 | 17535 | 22892 | 185 | | | | | | | | |
| 1701 | 22893 | 265 | KIX42365.1 | 17537 | 22894 | 185 | | | | | | | | |
| 1702 | 22895 | 265 | KIX63304.1 | 17539 | 22896 | 185 | | | | | | | | |
| 1703 | 22897 | 280 | CDU27412.1 | 17541 | 22898 | 185 | | | | | | | | |
| 1704 | 22899 | 265 | AJW52246.1 | 17543 | 22900 | 185 | | | | | | | | |
| 1705 | 22901 | 263 | AJX27503.1 | 17545 | 22902 | 185 | | | | | | | | |
| 1706 | 22903 | 273 | CRY48214.1 | 17547 | 22904 | 185 | | | | | | | | |
| 1707 | 22905 | 263 | CPN90172.1 | 17549 | 22906 | 185 | | | | | | | | |
| 1708 | 22907 | 263 | CPO07156.1 | 17551 | 22908 | 185 | | | | | | | | |
| 1709 | 22909 | 263 | CPM40598.1 | 17553 | 22910 | 185 | | | | | | | | |
| 1710 | 22911 | 263 | CPQ62540.1 | 17555 | 22912 | 185 | | | | | | | | |
| 1711 | 22913 | 263 | CPQ18506.1 | 17557 | 22914 | 185 | | | | | | | | |
| 1712 | 22915 | 263 | CPI70778.1 | 17559 | 22916 | 185 | | | | | | | | |
| 1713 | 22917 | 273 | CPK87974.1 | 17561 | 22918 | 185 | | | | | | | | |
| 1714 | 22919 | 263 | CPJ40996.1 | 17563 | 22920 | 185 | | | | | | | | |
| 1715 | 22921 | 263 | CPJ76845.1 | 17565 | 22922 | 185 | | | | | | | | |
| 1716 | 22923 | 263 | CPJ85529.1 | 17567 | 22924 | 185 | | | | | | | | |
| 1717 | 22925 | 263 | CPH77120.1 | 17569 | 22926 | 185 | | | | | | | | |
| 1718 | 22927 | 273 | CPJ25350.1 | 17571 | 22928 | 185 | | | | | | | | |
| 1719 | 22929 | 273 | CPR02274.1 | 17573 | 22930 | 185 | | | | | | | | |
| 1720 | 22931 | 273 | CPJ69705.1 | 17575 | 22932 | 185 | | | | | | | | |
| 1721 | 22933 | 273 | CPK70888.1 | 17577 | 22934 | 185 | | | | | | | | |
| 1722 | 22935 | 263 | CPQ22962.1 | 17579 | 22936 | 185 | | | | | | | | |
| 1723 | 22937 | 263 | CPN03567.1 | 17581 | 22938 | 185 | | | | | | | | |
| 1724 | 22939 | 263 | CPH78867.1 | 17583 | 22940 | 185 | | | | | | | | |
| 1725 | 22941 | 273 | CPI57666.1 | 17585 | 22942 | 185 | | | | | | | | |
| 1726 | 22943 | 263 | CPN26725.1 | 17587 | 22944 | 185 | | | | | | | | |
| 1727 | 22945 | 273 | CPK20278.1 | 17589 | 22946 | 185 | | | | | | | | |
| 1728 | 22947 | 273 | CPM33040.1 | 17591 | 22948 | 185 | | | | | | | | |
| 1729 | 22949 | 263 | CPN87647.1 | 17593 | 22950 | 185 | | | | | | | | |
| 1730 | 22951 | 263 | CPQ68085.1 | 17595 | 22952 | 185 | | | | | | | | |
| 1731 | 22953 | 273 | CPM53745.1 | 17597 | 22954 | 185 | | | | | | | | |
| 1732 | 22955 | 273 | CPM10536.1 | 17599 | 22956 | 185 | | | | | | | | |
| 1733 | 22957 | 273 | CPP09107.1 | 17601 | 22958 | 185 | | | | | | | | |
| 1734 | 22959 | 263 | CPL40913.1 | 17603 | 22960 | 185 | | | | | | | | |
| 1735 | 22961 | 263 | CPN14465.1 | 17605 | 22962 | 185 | | | | | | | | |
| 1736 | 22963 | 263 | CPK90364.1 | 17607 | 22964 | 185 | | | | | | | | |
| 1737 | 22965 | 273 | CPP68466.1 | 17609 | 22966 | 185 | | | | | | | | |
| 1738 | 22967 | 273 | CPP97961.1 | 17611 | 22968 | 185 | | | | | | | | |
| 1739 | 22969 | 273 | CPN87403.1 | 17613 | 22970 | 185 | | | | | | | | |
| 1740 | 22971 | 263 | CPR00114.1 | 17615 | 22972 | 185 | | | | | | | | |
| 1741 | 22973 | 273 | CPH39487.1 | 17617 | 22974 | 185 | | | | | | | | |
| 1742 | 22975 | 263 | CPG82791.1 | 17619 | 22976 | 185 | | | | | | | | |
| 1743 | 22977 | 263 | CPM46639.1 | 17621 | 22978 | 185 | | | | | | | | |
| 1744 | 22979 | 273 | CPG38295.1 | 17623 | 22980 | 185 | | | | | | | | |
| 1745 | 22981 | 273 | CPH00861.1 | 17625 | 22982 | 185 | | | | | | | | |
| 1746 | 22983 | 273 | CPH41999.1 | 17627 | 22984 | 185 | | | | | | | | |
| 1747 | 22985 | 265 | ALB92816.1 | 17629 | 22986 | 185 | | | | | | | | |
| 1748 | 22987 | 265 | ALB98878.1 | 17631 | 22988 | 185 | | | | | | | | |
| 1749 | 22989 | 265 | ALC58208.1 | 17633 | 22990 | 185 | | | | | | | | |
| 1750 | 22991 | 273 | CFT55150.1 | 17635 | 22992 | 185 | | | | | | | | |
| 1751 | 22993 | 273 | CPQ86657.1 | 17637 | 22994 | 185 | | | | | | | | |
| 1752 | 22995 | 263 | CFP14334.1 | 17639 | 22996 | 185 | | | | | | | | |
| 1753 | 22997 | 273 | CFU67051.1 | 17641 | 22998 | 185 | | | | | | | | |
| 1754 | 22999 | 263 | CFM98894.1 | 17643 | 23000 | 185 | | | | | | | | |
| 1755 | 23001 | 263 | CFM81861.1 | 17645 | 23002 | 185 | | | | | | | | |
| 1756 | 23003 | 273 | CPM85694.1 | 17647 | 23004 | 185 | | | | | | | | |
| 1757 | 23005 | 263 | CPL97321.1 | 17649 | 23006 | 185 | | | | | | | | |
| 1758 | 23007 | 273 | CFO92223.1 | 17651 | 23008 | 185 | | | | | | | | |
| 1759 | 23009 | 263 | CFN19257.1 | 17653 | 23010 | 185 | | | | | | | | |
| 1760 | 23011 | 263 | CPL11614.1 | 17655 | 23012 | 185 | | | | | | | | |
| 1761 | 23013 | 273 | CPN25033.1 | 17657 | 23014 | 185 | | | | | | | | |
| 1762 | 23015 | 263 | CPO46711.1 | 17659 | 23016 | 185 | | | | | | | | |
| 1763 | 23017 | 263 | CFL99477.1 | 17661 | 23018 | 185 | | | | | | | | |
| 1764 | 23019 | 263 | CPO89917.1 | 17663 | 23020 | 185 | | | | | | | | |
| 1765 | 23021 | 263 | CFW49157.1 | 17665 | 23022 | 185 | | | | | | | | |
| 1766 | 23023 | 273 | CFM22758.1 | 17667 | 23024 | 185 | | | | | | | | |
| 1767 | 23025 | 273 | CPK84381.1 | 17669 | 23026 | 185 | | | | | | | | |
| 1768 | 23027 | 263 | CFO79489.1 | 17671 | 23028 | 185 | | | | | | | | |
| 1769 | 23029 | 273 | CFU75865.1 | 17673 | 23030 | 185 | | | | | | | | |
| 1770 | 23031 | 273 | CFU18825.1 | 17675 | 23032 | 185 | | | | | | | | |
| 1771 | 23033 | 273 | CFM67596.1 | 17677 | 23034 | 185 | | | | | | | | |
| 1772 | 23035 | 273 | CFW79172.1 | 17679 | 23036 | 185 | | | | | | | | |
| 1773 | 23037 | 263 | CFO54183.1 | 17681 | 23038 | 185 | | | | | | | | |
| 1774 | 23039 | 263 | CFN47252.1 | 17683 | 23040 | 185 | | | | | | | | |
| 1775 | 23041 | 273 | CFU32913.1 | 17685 | 23042 | 185 | | | | | | | | |
| 1776 | 23043 | 263 | CFP26211.1 | 17687 | 23044 | 185 | | | | | | | | |
| 1777 | 23045 | 263 | CPP85412.1 | 17689 | 23046 | 185 | | | | | | | | |
| 1778 | 23047 | 273 | CFM37247.1 | 17691 | 23048 | 185 | | | | | | | | |
| 1779 | 23049 | 273 | CPQ63792.1 | 17693 | 23050 | 185 | | | | | | | | |
| 1780 | 23051 | 263 | CFO10821.1 | 17695 | 23052 | 185 | | | | | | | | |
| 1781 | 23053 | 273 | CPL75805.1 | 17697 | 23054 | 185 | | | | | | | | |
| 1782 | 23055 | 273 | CFM72559.1 | 17699 | 23056 | 185 | | | | | | | | |
| 1783 | 23057 | 273 | CPK97857.1 | 17701 | 23058 | 185 | | | | | | | | |
| 1784 | 23059 | 263 | CPN81591.1 | 17703 | 23060 | 185 | | | | | | | | |
| 1785 | 23061 | 273 | CFW09150.1 | 17705 | 23062 | 185 | | | | | | | | |
| 1786 | 23063 | 263 | CFU54704.1 | 17707 | 23064 | 185 | | | | | | | | |
| 1787 | 23065 | 263 | CFK50093.1 | 17709 | 23066 | 185 | | | | | | | | |
| 1788 | 23067 | 273 | CPG83454.1 | 17711 | 23068 | 185 | | | | | | | | |
| 1789 | 23069 | 273 | CFT72111.1 | 17713 | 23070 | 185 | | | | | | | | |
| 1790 | 23071 | 273 | CFL13096.1 | 17715 | 23072 | 185 | | | | | | | | |
| 1791 | 23073 | 273 | CFL88482.1 | 17717 | 23074 | 185 | | | | | | | | |
| 1792 | 23075 | 263 | CPE71771.1 | 17719 | 23076 | 185 | | | | | | | | |
| 1793 | 23077 | 273 | CFT96164.1 | 17721 | 23078 | 185 | | | | | | | | |
| 1794 | 23079 | 273 | CFT85443.1 | 17723 | 23080 | 185 | | | | | | | | |
| 1795 | 23081 | 263 | CFL56340.1 | 17725 | 23082 | 185 | | | | | | | | |
| 1796 | 23083 | 263 | CPI51266.1 | 17727 | 23084 | 185 | | | | | | | | |
| 1797 | 23085 | 249 | ESG20658.1 | 17729 | 23086 | 295 | | | | | | | | |
| 1798 | 23087 | 278 | AJQ64507.1 | 17731 | 23088 | 303 | | | | | | | | |
| 1799 | 23089 | 278 | AJZ80319.1 | 17733 | 23090 | 303 | | | | | | | | |
| 1800 | 23091 | 234 | CPW02870.1 | 17735 | 23092 | 170 | | | | | | | | |
| 1801 | 23093 | 279 | GAC48718.1 | 17737 | 23094 | 171 | | | | | | | | |
| 1802 | 23095 | 258 | ABM10182.1 | 17739 | 23096 | 169 | | | | | | | | |
| 1803 | 23097 | 285 | ERN46352.1 | 17741 | 23098 | 169 | | | | | | | | |
| 1804 | 23099 | 230 | KTD01947.1 | 17743 | 23100 | 185 | | | | | | | | |
| 1805 | 23101 | 297 | CCV65563.1 | 17745 | 23102 | 162 | | | | | | | | |
| 1806 | 23103 | 278 | KMK02864.1 | 17747 | 23104 | 169 | | | | | | | | |
| 1807 | 23105 | 276 | AHJ12535.1 | 17749 | 23106 | 183 | | | | | | | | |
| 1808 | 23107 | 281 | KUL42777.1 | 17751 | 23108 | 424 | | | | | | | | |
| 1809 | 23109 | 275 | EID21941.1 | 17753 | 23110 | 166 | | | | | | | | |
| 1810 | 23111 | 275 | GAD37766.1 | 17755 | 23112 | 166 | | | | | | | | |
| 1811 | 23113 | 279 | KRK83039.1 | 17757 | 23114 | 165 | | | | | | | | |
| 1812 | 23115 | 279 | EFV85441.1 | 17759 | 23116 | 186 | | | | | | | | |
| 1813 | 23117 | 273 | EMZ26177.1 | 17761 | 23118 | 202 | | | | | | | | |
| 1814 | 23119 | 290 | EKX62814.1 | 17763 | 23120 | 410 | | | | | | | | |
| 1815 | 23121 | 293 | ABR64201.1 | 17765 | 23122 | 166 | | | | | | | | |
| 1816 | 23123 | 292 | KND18806.1 | 17767 | 23124 | 166 | | | | | | | | |
| 1817 | 23125 | 285 | ADG41467.1 | 17769 | 23126 | 166 | | | | | | | | |
| 1818 | 23127 | 290 | EEA91363.1 | 17771 | 23128 | 168 | | | | | | | | |
| 1819 | 23129 | 284 | EHI61154.1 | 17773 | 23130 | 331 | | | | | | | | |
| 1820 | 23131 | 281 | EFF62518.1 | 17775 | 23132 | 181 | | | | | | | | |
| 1821 | 23133 | 276 | ALS25373.1 | 17777 | 23134 | 184 | | | | | | | | |
| 1822 | 23135 | 275 | KGM37270.1 | 17779 | 23136 | 166 | | | | | | | | |
| 1823 | 23137 | 293 | EWG70340.1 | 17781 | 23138 | 167 | | | | | | | | |
| 1824 | 23139 | 309 | AKP49794.1 | 17783 | 23140 | 167 | | | | | | | | |
| 1825 | 23141 | 207 | KJV29336.1 | 17785 | 23142 | 186 | | | | | | | | |
| 1826 | 23143 | 253 | EIL89964.1 | 17787 | 23144 | 125 | | | | | | | | |
| 1827 | 23145 | 291 | KFF05552.1 | 17789 | 23146 | 166 | | | | | | | | |
| 1828 | 23147 | 306 | CTQ72907.1 | 17791 | 23148 | 169 | | | | | | | | |
| 1829 | 23149 | 280 | KRU21992.1 | 17793 | 23150 | 188 | | | | | | | | |
| 1830 | 23151 | 284 | KJL32260.1 | 17795 | 23152 | 169 | | | | | | | | |
| 1831 | 23153 | 276 | EHS54841.1 | 17797 | 23154 | 183 | | | | | | | | |
| 1832 | 23155 | 291 | EJL63881.1 | 17799 | 23156 | 166 | | | | | | | | |
| 1833 | 23157 | 278 | EJL84706.1 | 17801 | 23158 | 166 | | | | | | | | |
| 1834 | 23159 | 309 | EID73850.1 | 17803 | 23160 | 167 | | | | | | | | |
| 1835 | 23161 | 280 | KWF17575.1 | 17805 | 23162 | 185 | | | | | | | | |
| 1836 | 23163 | 285 | ERI96810.1 | 17807 | 23164 | 215 | | | | | | | | |
| 1837 | 23165 | 319 | EOS38322.1 | 17809 | 23166 | 166 | | | | | | | | |
| 1838 | 23167 | 285 | EOS41469.1 | 17811 | 23168 | 202 | | | | | | | | |
| 1839 | 23169 | 271 | CDF26097.1 | 17813 | 23170 | 203 | | | | | | | | |
| 1840 | 23171 | 317 | KGN67553.1 | 17815 | 23172 | 170 | | | | | | | | |
| 1841 | 23173 | 277 | KUK53686.1 | 17817 | 23174 | 170 | | | | | | | | |
| 1842 | 23175 | 293 | KKO89197.1 | 17819 | 23176 | 183 | | | | | | | | |
| 1843 | 23177 | 278 | KNC08319.1 | 17821 | 23178 | 166 | | | | | | | | |
| 1844 | 23179 | 209 | KQN47926.1 | 17823 | 23180 | 188 | | | | | | | | |
| 1845 | 23181 | 276 | KKH38513.1 | 17825 | 23182 | 193 | | | | | | | | |
| 1846 | 23183 | 276 | KKH53103.1 | 17827 | 23184 | 193 | | | | | | | | |
| 1847 | 23185 | 276 | KKH68856.1 | 17829 | 23186 | 193 | | | | | | | | |
| 1848 | 23187 | 276 | KKH71867.1 | 17831 | 23188 | 193 | | | | | | | | |
| 1849 | 23189 | 276 | KKH80639.1 | 17833 | 23190 | 193 | | | | | | | | |
| 1850 | 23191 | 276 | KKF98497.1 | 17835 | 23192 | 193 | | | | | | | | |
| 1851 | 23193 | 276 | KKH51285.1 | 17837 | 23194 | 193 | | | | | | | | |
| 1852 | 23195 | 276 | KKH64072.1 | 17839 | 23196 | 193 | | | | | | | | |
| 1853 | 23197 | 276 | KKH76319.1 | 17841 | 23198 | 193 | | | | | | | | |
| 1854 | 23199 | 276 | KKH88480.1 | 17843 | 23200 | 193 | | | | | | | | |
| 1855 | 23201 | 276 | KKH75120.1 | 17845 | 23202 | 193 | | | | | | | | |
| 1856 | 23203 | 276 | KKH91068.1 | 17847 | 23204 | 193 | | | | | | | | |
| 1857 | 23205 | 276 | KKI00019.1 | 17849 | 23206 | 193 | | | | | | | | |
| 1858 | 23207 | 276 | KKI06937.1 | 17851 | 23208 | 193 | | | | | | | | |
| 1859 | 23209 | 276 | KKI04816.1 | 17853 | 23210 | 193 | | | | | | | | |
| 1860 | 23211 | 255 | ERH59125.1 | 17855 | 23212 | 190 | | | | | | | | |
| 1861 | 23213 | 279 | ADG31356.1 | 17857 | 23214 | 187 | ADG31358.1 | 17858 | 23215 | 186 | | | | |
| 1862 | 23216 | 273 | AKV63927.1 | 17860 | 23217 | 188 | AKV63929.1 | 17861 | 23218 | 231 | | | | |
| 1863 | 23219 | 281 | CDB59853.1 | 17863 | 23220 | 187 | CDB59854.1 | 17864 | 23221 | 170 | | | | |
| 1864 | 23222 | 297 | EDO56708.1 | 17866 | 23223 | 81 | EDO56706.1 | 17867 | 23224 | 184 | | | | |
| 1865 | 23225 | 271 | KGN82594.1 | 17869 | 23226 | 187 | KGN82592.1 | 17870 | 23227 | 224 | | | | |
| 1866 | 23228 | 277 | AKR49339.1 | 17872 | 23229 | 183 | AKR49342.1 | 17873 | 23230 | 169 | | | | |
| 1867 | 23231 | 292 | ESS57196.1 | 17875 | 23232 | 304 | ESS57523.1 | 17876 | 23233 | 183 | | | | |
| 1868 | 23234 | 278 | AAN80857.1 | 17878 | 23235 | 307 | AAN80858.1 | 17879 | 23236 | 169 | | | | |
| 1869 | 23237 | 278 | EFJ93883.1 | 17881 | 23238 | 307 | EFJ93882.1 | 17882 | 23239 | 169 | | | | |
| 1870 | 23240 | 278 | EFU46135.1 | 17884 | 23241 | 307 | EFU46134.1 | 17885 | 23242 | 169 | | | | |
| 1871 | 23243 | 278 | EFU49593.1 | 17887 | 23244 | 307 | EFU49594.1 | 17888 | 23245 | 169 | | | | |
| 1872 | 23246 | 278 | EFJ59504.1 | 17890 | 23247 | 307 | EFJ59503.1 | 17891 | 23248 | 169 | | | | |
| 1873 | 23249 | 278 | EGB82663.1 | 17893 | 23250 | 307 | EGB82664.1 | 17894 | 23251 | 169 | | | | |
| 1874 | 23252 | 278 | EFM52808.1 | 17896 | 23253 | 307 | EFM52807.1 | 17897 | 23254 | 169 | | | | |
| 1875 | 23255 | 278 | EGB52116.1 | 17899 | 23256 | 287 | EGB52117.1 | 17900 | 23257 | 169 | | | | |
| 1876 | 23258 | 278 | AER84778.1 | 17902 | 23259 | 307 | AER84779.1 | 17903 | 23260 | 169 | | | | |
| 1877 | 23261 | 278 | AER89697.1 | 17905 | 23262 | 307 | AER89698.1 | 17906 | 23263 | 169 | | | | |
| 1878 | 23264 | 278 | EHO02521.1 | 17908 | 23265 | 307 | EHO02520.1 | 17909 | 23266 | 169 | | | | |
| 1879 | 23267 | 278 | ELL42846.1 | 17911 | 23268 | 287 | ELL42847.1 | 17912 | 23269 | 169 | | | | |
| 1880 | 23270 | 278 | ELI13534.1 | 17914 | 23271 | 307 | ELI13535.1 | 17915 | 23272 | 169 | | | | |
| 1881 | 23273 | 278 | ELI43904.1 | 17917 | 23274 | 307 | ELI43905.1 | 17918 | 23275 | 169 | | | | |
| 1882 | 23276 | 278 | ELI67624.1 | 17920 | 23277 | 307 | ELI67625.1 | 17921 | 23278 | 169 | | | | |
| 1883 | 23279 | 278 | ELI72587.1 | 17923 | 23280 | 307 | ELI72588.1 | 17924 | 23281 | 169 | | | | |
| 1884 | 23282 | 278 | ELI75958.1 | 17926 | 23283 | 307 | ELI75959.1 | 17927 | 23284 | 169 | | | | |
| 1885 | 23285 | 278 | ELJ03559.1 | 17929 | 23286 | 307 | ELJ03560.1 | 17930 | 23287 | 169 | | | | |
| 1886 | 23288 | 278 | ELJ14332.1 | 17932 | 23289 | 307 | ELJ14333.1 | 17933 | 23290 | 169 | | | | |
| 1887 | 23291 | 278 | ELJ38403.1 | 17935 | 23292 | 307 | ELJ38404.1 | 17936 | 23293 | 169 | | | | |
| 1888 | 23294 | 278 | ELJ71311.1 | 17938 | 23295 | 307 | ELJ71312.1 | 17939 | 23296 | 169 | | | | |
| 1889 | 23297 | 278 | ELI89430.1 | 17941 | 23298 | 307 | ELI89431.1 | 17942 | 23299 | 169 | | | | |
| 1890 | 23300 | 278 | ELJ28708.1 | 17944 | 23301 | 307 | ELJ28709.1 | 17945 | 23302 | 169 | | | | |
| 1891 | 23303 | 278 | ELC30029.1 | 17947 | 23304 | 307 | ELC30030.1 | 17948 | 23305 | 169 | | | | |
| 1892 | 23306 | 278 | ELC84582.1 | 17950 | 23307 | 307 | ELC84583.1 | 17951 | 23308 | 169 | | | | |
| 1893 | 23309 | 278 | ELC89742.1 | 17953 | 23310 | 307 | ELC89743.1 | 17954 | 23311 | 169 | | | | |
| 1894 | 23312 | 278 | ELC99566.1 | 17956 | 23313 | 307 | ELC99567.1 | 17957 | 23314 | 169 | | | | |
| 1895 | 23315 | 278 | ELD14725.1 | 17959 | 23316 | 307 | ELD14726.1 | 17960 | 23317 | 169 | | | | |
| 1896 | 23318 | 278 | ELD50498.1 | 17962 | 23319 | 307 | ELD50499.1 | 17963 | 23320 | 169 | | | | |
| 1897 | 23321 | 278 | ELE05783.1 | 17965 | 23322 | 307 | ELE05784.1 | 17966 | 23323 | 169 | | | | |
| 1898 | 23324 | 278 | ELE19655.1 | 17968 | 23325 | 307 | ELE19656.1 | 17969 | 23326 | 169 | | | | |
| 1899 | 23327 | 278 | ELE23558.1 | 17971 | 23328 | 307 | ELE23559.1 | 17972 | 23329 | 169 | | | | |
| 1900 | 23330 | 278 | ELE50399.1 | 17974 | 23331 | 307 | ELE50400.1 | 17975 | 23332 | 169 | | | | |
| 1901 | 23333 | 278 | ELE80390.1 | 17977 | 23334 | 307 | ELE80391.1 | 17978 | 23335 | 169 | | | | |
| 1902 | 23336 | 278 | ELE90660.1 | 17980 | 23337 | 307 | ELE90661.1 | 17981 | 23338 | 169 | | | | |
| 1903 | 23339 | 278 | ELE91476.1 | 17983 | 23340 | 307 | ELE91477.1 | 17984 | 23341 | 169 | | | | |
| 1904 | 23342 | 278 | ELF50403.1 | 17986 | 23343 | 307 | ELF50404.1 | 17987 | 23344 | 169 | | | | |
| 1905 | 23345 | 278 | ELF92088.1 | 17989 | 23346 | 307 | ELF92089.1 | 17990 | 23347 | 169 | | | | |
| 1906 | 23348 | 278 | ELG14336.1 | 17992 | 23349 | 307 | ELG14337.1 | 17993 | 23350 | 169 | | | | |
| 1907 | 23351 | 278 | ELG17001.1 | 17995 | 23352 | 307 | ELG17002.1 | 17996 | 23353 | 169 | | | | |
| 1908 | 23354 | 278 | ELG24589.1 | 17998 | 23355 | 307 | ELG24590.1 | 17999 | 23356 | 169 | | | | |
| 1909 | 23357 | 278 | ELG54536.1 | 18001 | 23358 | 307 | ELG54537.1 | 18002 | 23359 | 169 | | | | |
| 1910 | 23360 | 278 | ELG58276.1 | 18004 | 23361 | 307 | ELG58277.1 | 18005 | 23362 | 169 | | | | |
| 1911 | 23363 | 278 | ELH44354.1 | 18007 | 23364 | 307 | ELH44355.1 | 18008 | 23365 | 169 | | | | |
| 1912 | 23366 | 278 | ELH62527.1 | 18010 | 23367 | 307 | ELH62528.1 | 18011 | 23368 | 169 | | | | |
| 1913 | 23369 | 278 | ELH59266.1 | 18013 | 23370 | 307 | ELH59267.1 | 18014 | 23371 | 169 | | | | |
| 1914 | 23372 | 278 | ELH80075.1 | 18016 | 23373 | 307 | ELH80076.1 | 18017 | 23374 | 169 | | | | |
| 1915 | 23375 | 278 | ELH85628.1 | 18019 | 23376 | 307 | ELH85629.1 | 18020 | 23377 | 169 | | | | |
| 1916 | 23378 | 278 | ELH88469.1 | 18022 | 23379 | 307 | ELH88470.1 | 18023 | 23380 | 169 | | | | |
| 1917 | 23381 | 278 | ELI08745.1 | 18025 | 23382 | 307 | ELI08746.1 | 18026 | 23383 | 169 | | | | |
| 1918 | 23384 | 278 | EOR52538.1 | 18028 | 23385 | 307 | EOR52539.1 | 18029 | 23386 | 169 | | | | |
| 1919 | 23387 | 278 | EOV07666.1 | 18031 | 23388 | 307 | EOV07667.1 | 18032 | 23389 | 169 | | | | |
| 1920 | 23390 | 278 | EOX10650.1 | 18034 | 23391 | 307 | EOX10651.1 | 18035 | 23392 | 169 | | | | |
| 1921 | 23393 | 278 | EOV94184.1 | 18037 | 23394 | 307 | EOV94185.1 | 18038 | 23395 | 169 | | | | |
| 1922 | 23396 | 278 | EQN11055.1 | 18040 | 23397 | 307 | EQN11054.1 | 18041 | 23398 | 169 | | | | |
| 1923 | 23399 | 278 | EQN19683.1 | 18043 | 23400 | 307 | EQN19684.1 | 18044 | 23401 | 169 | | | | |
| 1924 | 23402 | 278 | EQN35017.1 | 18046 | 23403 | 307 | EQN35018.1 | 18047 | 23404 | 140 | | | | |
| 1925 | 23405 | 278 | EQN48987.1 | 18049 | 23406 | 307 | EQN48988.1 | 18050 | 23407 | 169 | | | | |
| 1926 | 23408 | 278 | EQN62514.1 | 18052 | 23409 | 307 | EQN62515.1 | 18053 | 23410 | 169 | | | | |
| 1927 | 23411 | 278 | EQN79329.1 | 18055 | 23412 | 307 | EQN79330.1 | 18056 | 23413 | 169 | | | | |
| 1928 | 23414 | 278 | EQN86133.1 | 18058 | 23415 | 307 | EQN86134.1 | 18059 | 23416 | 169 | | | | |
| 1929 | 23417 | 278 | EQN96593.1 | 18061 | 23418 | 307 | EQN96594.1 | 18062 | 23419 | 169 | | | | |
| 1930 | 23420 | 278 | EQO12275.1 | 18064 | 23421 | 307 | EQO12274.1 | 18065 | 23422 | 169 | | | | |
| 1931 | 23423 | 278 | EQO16636.1 | 18067 | 23424 | 307 | EQO16637.1 | 18068 | 23425 | 169 | | | | |
| 1932 | 23426 | 278 | EQO18513.1 | 18070 | 23427 | 307 | EQO18514.1 | 18071 | 23428 | 169 | | | | |
| 1933 | 23429 | 278 | EQO32109.1 | 18073 | 23430 | 307 | EQO32110.1 | 18074 | 23431 | 169 | | | | |
| 1934 | 23432 | 278 | EQO40645.1 | 18076 | 23433 | 307 | EQO40646.1 | 18077 | 23434 | 169 | | | | |
| 1935 | 23435 | 278 | EQO47385.1 | 18079 | 23436 | 307 | EQO47386.1 | 18080 | 23437 | 169 | | | | |
| 1936 | 23438 | 278 | EQO84678.1 | 18082 | 23439 | 307 | EQO84679.1 | 18083 | 23440 | 169 | | | | |
| 1937 | 23441 | 278 | EQO93034.1 | 18085 | 23442 | 307 | EQO93035.1 | 18086 | 23443 | 169 | | | | |
| 1938 | 23444 | 278 | EQO00511.1 | 18088 | 23445 | 307 | EQP00512.1 | 18089 | 23446 | 169 | | | | |
| 1939 | 23447 | 278 | EQP09248.1 | 18091 | 23448 | 307 | EQP09249.1 | 18092 | 23449 | 169 | | | | |
| 1940 | 23450 | 278 | EQP11536.1 | 18094 | 23451 | 307 | EQP11537.1 | 18095 | 23452 | 169 | | | | |
| 1941 | 23453 | 278 | EQP21161.1 | 18097 | 23454 | 307 | EQP21162.1 | 18098 | 23455 | 169 | | | | |
| 1942 | 23456 | 278 | EQP38128.1 | 18100 | 23457 | 307 | EQP38129.1 | 18101 | 23458 | 169 | | | | |
| 1943 | 23459 | 278 | EQP55411.1 | 18103 | 23460 | 307 | EQP55412.1 | 18104 | 23461 | 169 | | | | |
| 1944 | 23462 | 278 | EQP53331.1 | 18106 | 23463 | 307 | EQP53332.1 | 18107 | 23464 | 169 | | | | |
| 1945 | 23465 | 278 | EQP61512.1 | 18109 | 23466 | 307 | EQP61513.1 | 18110 | 23467 | 169 | | | | |
| 1946 | 23468 | 278 | EQP73528.1 | 18112 | 23469 | 307 | EQP73529.1 | 18113 | 23470 | 169 | | | | |
| 1947 | 23471 | 278 | EQP70427.1 | 18115 | 23472 | 307 | EQP70428.1 | 18116 | 23473 | 169 | | | | |
| 1948 | 23474 | 278 | EQQ06478.1 | 18118 | 23475 | 307 | EQQ06479.1 | 18119 | 23476 | 169 | | | | |
| 1949 | 23477 | 278 | EQQ25250.1 | 18121 | 23478 | 307 | EQQ25251.1 | 18122 | 23479 | 169 | | | | |
| 1950 | 23480 | 278 | EQQ27931.1 | 18124 | 23481 | 307 | EQQ27932.1 | 18125 | 23482 | 169 | | | | |
| 1951 | 23483 | 278 | EQQ41430.1 | 18127 | 23484 | 307 | EQQ41431.1 | 18128 | 23485 | 169 | | | | |
| 1952 | 23486 | 278 | EQQ33645.1 | 18130 | 23487 | 307 | EQQ33646.1 | 18131 | 23488 | 169 | | | | |
| 1953 | 23489 | 278 | EQQ53132.1 | 18133 | 23490 | 307 | EQQ53133.1 | 18134 | 23491 | 169 | | | | |
| 1954 | 23492 | 278 | EQQ74420.1 | 18136 | 23493 | 307 | EQQ74421.1 | 18137 | 23494 | 169 | | | | |
| 1955 | 23495 | 278 | EQQ75979.1 | 18139 | 23496 | 307 | EQQ75980.1 | 18140 | 23497 | 169 | | | | |
| 1956 | 23498 | 278 | EQQ88666.1 | 18142 | 23499 | 307 | EQQ88667.1 | 18143 | 23500 | 169 | | | | |
| 1957 | 23501 | 278 | EQQ90543.1 | 18145 | 23502 | 307 | EQQ90544.1 | 18146 | 23503 | 169 | | | | |
| 1958 | 23504 | 278 | EQR07408.1 | 18148 | 23505 | 307 | EQR07409.1 | 18149 | 23506 | 169 | | | | |
| 1959 | 23507 | 278 | EQR15136.1 | 18151 | 23508 | 307 | EQR15137.1 | 18152 | 23509 | 169 | | | | |
| 1960 | 23510 | 278 | EQR19369.1 | 18154 | 23511 | 307 | EQR19370.1 | 18155 | 23512 | 169 | | | | |
| 1961 | 23513 | 278 | EQR28928.1 | 18157 | 23514 | 307 | EQR28929.1 | 18158 | 23515 | 169 | | | | |
| 1962 | 23516 | 278 | EQR67052.1 | 18160 | 23517 | 307 | EQR67053.1 | 18161 | 23518 | 169 | | | | |
| 1963 | 23519 | 278 | EQR88162.1 | 18163 | 23520 | 307 | EQR88163.1 | 18164 | 23521 | 169 | | | | |
| 1964 | 23522 | 278 | EQR94356.1 | 18166 | 23523 | 307 | EQR94357.1 | 18167 | 23524 | 169 | | | | |
| 1965 | 23525 | 278 | EQS19448.1 | 18169 | 23526 | 307 | EQS19449.1 | 18170 | 23527 | 169 | | | | |
| 1966 | 23528 | 278 | EQS13122.1 | 18172 | 23529 | 307 | EQS13123.1 | 18173 | 23530 | 169 | | | | |
| 1967 | 23531 | 278 | EQS20957.1 | 18175 | 23532 | 307 | EQS20958.1 | 18176 | 23533 | 169 | | | | |
| 1968 | 23534 | 278 | EQS40212.1 | 18178 | 23535 | 307 | EQS40213.1 | 18179 | 23536 | 169 | | | | |
| 1969 | 23537 | 278 | ERA74999.1 | 18181 | 23538 | 307 | ERA75000.1 | 18182 | 23539 | 169 | | | | |
| 1970 | 23540 | 278 | EQS62806.1 | 18184 | 23541 | 307 | EQS62807.1 | 18185 | 23542 | 169 | | | | |
| 1971 | 23543 | 278 | ERA80455.1 | 18187 | 23544 | 307 | ERA80456.1 | 18188 | 23545 | 169 | | | | |
| 1972 | 23546 | 278 | ERA86691.1 | 18190 | 23547 | 307 | ERA86692.1 | 18191 | 23548 | 169 | | | | |
| 1973 | 23549 | 278 | EQS67054.1 | 18193 | 23550 | 307 | EQS67055.1 | 18194 | 23551 | 169 | | | | |
| 1974 | 23552 | 278 | EQS96895.1 | 18196 | 23553 | 307 | EQS96896.1 | 18197 | 23554 | 169 | | | | |
| 1975 | 23555 | 278 | EQT00424.1 | 18199 | 23556 | 307 | EQT00425.1 | 18200 | 23557 | 169 | | | | |
| 1976 | 23558 | 278 | EQT14972.1 | 18202 | 23559 | 307 | EQT14973.1 | 18203 | 23560 | 169 | | | | |
| 1977 | 23561 | 278 | EQT25529.1 | 18205 | 23562 | 307 | EQT25530.1 | 18206 | 23563 | 169 | | | | |
| 1978 | 23564 | 278 | EQT45490.1 | 18208 | 23565 | 307 | EQT45491.1 | 18209 | 23566 | 169 | | | | |
| 1979 | 23567 | 278 | EQT50679.1 | 18211 | 23568 | 307 | EQT50680.1 | 18212 | 23569 | 169 | | | | |
| 1980 | 23570 | 278 | EQT83219.1 | 18214 | 23571 | 307 | EQT83220.1 | 18215 | 23572 | 169 | | | | |
| 1981 | 23573 | 249 | EQT95716.1 | 18217 | 23574 | 307 | EQT95717.1 | 18218 | 23575 | 169 | | | | |
| 1982 | 23576 | 278 | EQU21867.1 | 18220 | 23577 | 307 | EQU21868.1 | 18221 | 23578 | 169 | | | | |
| 1983 | 23579 | 278 | EQU12096.1 | 18223 | 23580 | 307 | EQU12097.1 | 18224 | 23581 | 169 | | | | |
| 1984 | 23582 | 278 | ERA90776.1 | 18226 | 23583 | 307 | ERA90777.1 | 18227 | 23584 | 169 | | | | |
| 1985 | 23585 | 278 | EQU70204.1 | 18229 | 23586 | 307 | EQU70205.1 | 18230 | 23587 | 169 | | | | |
| 1986 | 23588 | 278 | EQU77944.1 | 18232 | 23589 | 307 | EQU77945.1 | 18233 | 23590 | 169 | | | | |
| 1987 | 23591 | 278 | EQU95024.1 | 18235 | 23592 | 307 | EQU95025.1 | 18236 | 23593 | 169 | | | | |
| 1988 | 23594 | 278 | EQV01573.1 | 18238 | 23595 | 307 | EQV01574.1 | 18239 | 23596 | 169 | | | | |
| 1989 | 23597 | 278 | EQV09454.1 | 18241 | 23598 | 307 | EQV09455.1 | 18242 | 23599 | 169 | | | | |
| 1990 | 23600 | 278 | EQV27902.1 | 18244 | 23601 | 307 | EQV27903.1 | 18245 | 23602 | 169 | | | | |
| 1991 | 23603 | 278 | ERA94626.1 | 18247 | 23604 | 307 | ERA94627.1 | 18248 | 23605 | 169 | | | | |
| 1992 | 23606 | 278 | ERB07222.1 | 18250 | 23607 | 307 | ERB07223.1 | 18251 | 23608 | 169 | | | | |
| 1993 | 23609 | 278 | EQV33720.1 | 18253 | 23610 | 307 | EQV33721.1 | 18254 | 23611 | 169 | | | | |
| 1994 | 23612 | 278 | EQV70970.1 | 18256 | 23613 | 307 | EQV70971.1 | 18257 | 23614 | 169 | | | | |
| 1995 | 23615 | 278 | EQV84932.1 | 18259 | 23616 | 307 | EQV84933.1 | 18260 | 23617 | 169 | | | | |
| 1996 | 23618 | 278 | EQW43213.1 | 18262 | 23619 | 307 | EQW43214.1 | 18263 | 23620 | 169 | | | | |
| 1997 | 23621 | 278 | EQW70391.1 | 18265 | 23622 | 307 | EQW70392.1 | 18266 | 23623 | 169 | | | | |
| 1998 | 23624 | 278 | EQW80281.1 | 18268 | 23625 | 307 | EQW80282.1 | 18269 | 23626 | 169 | | | | |
| 1999 | 23627 | 278 | EQW87484.1 | 18271 | 23628 | 307 | EQW87485.1 | 18272 | 23629 | 169 | | | | |
| 2000 | 23630 | 278 | EQX12314.1 | 18274 | 23631 | 307 | EQX12315.1 | 18275 | 23632 | 169 | | | | |
| 2001 | 23633 | 278 | EQX35783.1 | 18277 | 23634 | 307 | EQX35784.1 | 18278 | 23635 | 169 | | | | |
| 2002 | 23636 | 278 | EQX43606.1 | 18280 | 23637 | 307 | EQX43607.1 | 18281 | 23638 | 169 | | | | |
| 2003 | 23639 | 278 | EQX66167.1 | 18283 | 23640 | 307 | EQX66168.1 | 18284 | 23641 | 169 | | | | |
| 2004 | 23642 | 278 | EQX76185.1 | 18286 | 23643 | 307 | EQX76186.1 | 18287 | 23644 | 169 | | | | |
| 2005 | 23645 | 278 | EQY22297.1 | 18289 | 23646 | 307 | EQY22298.1 | 18290 | 23647 | 169 | | | | |
| 2006 | 23648 | 278 | EQY33194.1 | 18292 | 23649 | 307 | EQY33195.1 | 18293 | 23650 | 169 | | | | |
| 2007 | 23651 | 278 | EQY45030.1 | 18295 | 23652 | 307 | EQY45031.1 | 18296 | 23653 | 169 | | | | |
| 2008 | 23654 | 278 | EQY55712.1 | 18298 | 23655 | 307 | EQY55713.1 | 18299 | 23656 | 169 | | | | |
| 2009 | 23657 | 278 | EQY72169.1 | 18301 | 23658 | 307 | EQY72170.1 | 18302 | 23659 | 169 | | | | |
| 2010 | 23660 | 278 | EQZ19656.1 | 18304 | 23661 | 307 | EQZ19657.1 | 18305 | 23662 | 169 | | | | |
| 2011 | 23663 | 278 | EQZ26677.1 | 18307 | 23664 | 307 | EQZ26676.1 | 18308 | 23665 | 169 | | | | |
| 2012 | 23666 | 278 | EQZ41388.1 | 18310 | 23667 | 307 | EQZ41389.1 | 18311 | 23668 | 169 | | | | |
| 2013 | 23669 | 278 | EQZ91635.1 | 18313 | 23670 | 307 | EQZ91636.1 | 18314 | 23671 | 169 | | | | |
| 2014 | 23672 | 278 | ERA32821.1 | 18316 | 23673 | 307 | ERA32822.1 | 18317 | 23674 | 169 | | | | |
| 2015 | 23675 | 278 | ERA36346.1 | 18319 | 23676 | 307 | ERA36347.1 | 18320 | 23677 | 169 | | | | |
| 2016 | 23678 | 278 | ERF54132.1 | 18322 | 23679 | 307 | ERF54133.1 | 18323 | 23680 | 169 | | | | |
| 2017 | 23681 | 278 | ESK18130.1 | 18325 | 23682 | 307 | ESK18131.1 | 18326 | 23683 | 169 | | | | |
| 2018 | 23684 | 278 | ESK18552.1 | 18328 | 23685 | 307 | ESK18553.1 | 18329 | 23686 | 169 | | | | |
| 2019 | 23687 | 278 | ESK29584.1 | 18331 | 23688 | 307 | ESK29585.1 | 18332 | 23689 | 169 | | | | |
| 2020 | 23690 | 278 | ESC95629.1 | 18334 | 23691 | 307 | ESC95630.1 | 18335 | 23692 | 169 | | | | |
| 2021 | 23693 | 278 | ESD43647.1 | 18337 | 23694 | 81 | ESD43646.1 | 18338 | 23695 | 169 | | | | |
| 2022 | 23696 | 278 | ESE27408.1 | 18340 | 23697 | 307 | ESE27409.1 | 18341 | 23698 | 169 | | | | |
| 2023 | 23699 | 278 | ESE32329.1 | 18343 | 23700 | 307 | ESE32328.1 | 18344 | 23701 | 169 | | | | |
| 2024 | 23702 | 278 | ESE38126.1 | 18346 | 23703 | 307 | ESE38127.1 | 18347 | 23704 | 169 | | | | |
| 2025 | 23705 | 278 | ESP10884.1 | 18349 | 23706 | 307 | ESP10883.1 | 18350 | 23707 | 169 | | | | |
| 2026 | 23708 | 278 | ESP23169.1 | 18352 | 23709 | 307 | ESP23170.1 | 18353 | 23710 | 169 | | | | |
| 2027 | 23711 | 278 | ETE11167.1 | 18355 | 23712 | 307 | ETE11168.1 | 18356 | 23713 | 169 | | | | |
| 2028 | 23714 | 278 | ETF35982.1 | 18358 | 23715 | 307 | ETF35983.1 | 18359 | 23716 | 169 | | | | |
| 2029 | 23717 | 278 | EZQ63562.1 | 18361 | 23718 | 307 | EZQ63563.1 | 18362 | 23719 | 169 | | | | |
| 2030 | 23720 | 278 | KDG03092.1 | 18364 | 23721 | 307 | KDG03093.1 | 18365 | 23722 | 169 | | | | |
| 2031 | 23723 | 278 | KDO90446.1 | 18367 | 23724 | 307 | KDO90447.1 | 18368 | 23725 | 169 | | | | |
| 2032 | 23726 | 278 | AID79059.1 | 18370 | 23727 | 307 | AID79060.1 | 18371 | 23728 | 169 | | | | |
| 2033 | 23729 | 278 | AIL14919.1 | 18373 | 23730 | 307 | AIL14831.1 | 18374 | 23731 | 169 | | | | |
| 2034 | 23732 | 278 | KLX17103.1 | 18376 | 23733 | 307 | KLX17104.1 | 18377 | 23734 | 169 | | | | |
| 2035 | 23735 | 278 | KLX44350.1 | 18379 | 23736 | 307 | KLX44351.1 | 18380 | 23737 | 169 | | | | |
| 2036 | 23738 | 278 | KLX45651.1 | 18382 | 23739 | 307 | KLX45652.1 | 18383 | 23740 | 169 | | | | |
| 2037 | 23741 | 278 | ALD37596.1 | 18385 | 23742 | 287 | ALD34923.1 | 18386 | 23743 | 169 | | | | |
| 2038 | 23744 | 278 | KSY03096.1 | 18388 | 23745 | 287 | KSY02660.1 | 18389 | 23746 | 169 | | | | |
| 2039 | 23747 | 278 | KUR38293.1 | 18391 | 23748 | 287 | KUR38255.1 | 18392 | 23749 | 169 | | | | |
| 2040 | 23750 | 278 | KUR81992.1 | 18394 | 23751 | 287 | KUR81833.1 | 18395 | 23752 | 169 | | | | |
| 2041 | 23753 | 278 | KUR94139.1 | 18397 | 23754 | 287 | KUR94070.1 | 18398 | 23755 | 169 | | | | |
| 2042 | 23756 | 278 | KUR99652.1 | 18400 | 23757 | 287 | KUR99611.1 | 18401 | 23758 | 169 | | | | |
| 2043 | 23759 | 278 | KUS68975.1 | 18403 | 23760 | 287 | KUS68706.1 | 18404 | 23761 | 169 | | | | |
| 2044 | 23762 | 278 | KUS65771.1 | 18406 | 23763 | 287 | KUS65753.1 | 18407 | 23764 | 169 | | | | |
| 2045 | 23765 | 278 | KUS06605.1 | 18409 | 23766 | 287 | KUS06587.1 | 18410 | 23767 | 169 | | | | |
| 2046 | 23768 | 278 | KUW86276.1 | 18412 | 23769 | 287 | KUW86272.1 | 18413 | 23770 | 169 | | | | |
| 2047 | 23771 | 278 | KUT33564.1 | 18415 | 23772 | 287 | KUT33560.1 | 18416 | 23773 | 169 | | | | |
| 2048 | 23774 | 278 | KUV71943.1 | 18418 | 23775 | 287 | KUV71917.1 | 18419 | 23776 | 169 | | | | |
| 2049 | 23777 | 278 | KUT65778.1 | 18421 | 23778 | 287 | KUT65774.1 | 18422 | 23779 | 169 | | | | |
| 2050 | 23780 | 278 | KUT92675.1 | 18424 | 23781 | 287 | KUT92653.1 | 18425 | 23782 | 169 | | | | |
| 2051 | 23783 | 278 | KUV55803.1 | 18427 | 23784 | 287 | KUV55765.1 | 18428 | 23785 | 169 | | | | |
| 2052 | 23786 | 278 | KUU93006.1 | 18430 | 23787 | 287 | KUU92986.1 | 18431 | 23788 | 169 | | | | |
| 2053 | 23789 | 278 | KUV32585.1 | 18433 | 23790 | 287 | KUV32575.1 | 18434 | 23791 | 169 | | | | |
| 2054 | 23792 | 278 | KUX05541.1 | 18436 | 23793 | 287 | KUX05522.1 | 18437 | 23794 | 169 | | | | |
| 2055 | 23795 | 278 | KUX02226.1 | 18439 | 23796 | 287 | KUX02163.1 | 18440 | 23797 | 169 | | | | |
| 2056 | 23798 | 278 | KUW18250.1 | 18442 | 23799 | 287 | KUW18189.1 | 18443 | 23800 | 169 | | | | |
| 2057 | 23801 | 278 | KUU04918.1 | 18445 | 23802 | 287 | KUU04901.1 | 18446 | 23803 | 169 | | | | |
| 2058 | 23804 | 278 | KUW44263.1 | 18448 | 23805 | 287 | KUW44251.1 | 18449 | 23806 | 169 | | | | |
| 2059 | 23807 | 278 | KUW29429.1 | 18451 | 23808 | 287 | KUW29414.1 | 18452 | 23809 | 169 | | | | |
| 2060 | 23810 | 278 | KUT58705.1 | 18454 | 23811 | 287 | KUT58680.1 | 18455 | 23812 | 169 | | | | |
| 2061 | 23813 | 278 | KUX37247.1 | 18457 | 23814 | 287 | KUX37209.1 | 18458 | 23815 | 169 | | | | |
| 2062 | 23816 | 278 | KUV14431.1 | 18460 | 23817 | 287 | KUV14409.1 | 18461 | 23818 | 169 | | | | |
| 2063 | 23819 | 278 | KUX73675.1 | 18463 | 23820 | 287 | KUX73638.1 | 18464 | 23821 | 169 | | | | |
| 2064 | 23822 | 278 | KUW86808.1 | 18466 | 23823 | 287 | KUW86784.1 | 18467 | 23824 | 169 | | | | |
| 2065 | 23825 | 278 | KUX51137.1 | 18469 | 23826 | 287 | KUX51101.1 | 18470 | 23827 | 169 | | | | |
| 2066 | 23828 | 278 | KVI42459.1 | 18472 | 23829 | 287 | KVI42423.1 | 18473 | 23830 | 169 | | | | |
| 2067 | 23831 | 278 | KVI22812.1 | 18475 | 23832 | 287 | KVI22807.1 | 18476 | 23833 | 169 | | | | |
| 2068 | 23834 | 278 | KXG92286.1 | 18478 | 23835 | 307 | KXG92285.1 | 18479 | 23836 | 169 | | | | |
| 2069 | 23837 | 278 | KXL04892.1 | 18481 | 23838 | 287 | KXL04868.1 | 18482 | 23839 | 169 | | | | |
| 2070 | 23840 | 278 | KXL27416.1 | 18484 | 23841 | 287 | KXL27377.1 | 18485 | 23842 | 169 | | | | |
| 2071 | 23843 | 278 | KXL05688.1 | 18487 | 23844 | 287 | KXL05684.1 | 18488 | 23845 | 169 | | | | |
| 2072 | 23846 | 278 | KXL13376.1 | 18490 | 23847 | 287 | KXL13116.1 | 18491 | 23848 | 169 | | | | |
| 2073 | 23849 | 278 | KTK78308.1 | 18493 | 23850 | 287 | KTK75706.1 | 18494 | 23851 | 169 | | | | |
| 2074 | 23852 | 285 | EPI32469.1 | 18496 | 23853 | 474 | EPI32467.1 | 18497 | 23854 | 153 | | | | |
| 2075 | 23855 | 283 | ETU24521.1 | 18499 | 23856 | 281 | ETU24519.1 | 18500 | 23857 | 173 | | | | |
| 2076 | 23858 | 281 | ELB67023.1 | 18502 | 23859 | 277 | ELB67021.1 | 18503 | 23860 | 166 | | | | |
| 2077 | 23861 | 271 | ENZ13054.1 | 18505 | 23862 | 98 | ENZ13056.1 | 18506 | 23863 | 134 | | | | |
| 2078 | 23864 | 278 | AHW13888.1 | 18508 | 23865 | 294 | AHW13889.1 | 18509 | 23866 | 169 | | | | |
| 2079 | 23867 | 278 | ESP72123.1 | 18511 | 23868 | 295 | ESP72122.1 | 18512 | 23869 | 169 | | | | |
| 2080 | 23870 | 278 | ESP76606.1 | 18514 | 23871 | 295 | ESP76605.1 | 18515 | 23872 | 169 | | | | |
| 2081 | 23873 | 278 | KFT73589.1 | 18517 | 23874 | 303 | KFT73590.1 | 18518 | 23875 | 169 | | | | |
| 2082 | 23876 | 278 | KHP33250.1 | 18520 | 23877 | 287 | KHP33098.1 | 18521 | 23878 | 169 | | | | |
| 2083 | 23879 | 278 | KHP35541.1 | 18523 | 23880 | 287 | KHP34816.1 | 18524 | 23881 | 169 | | | | |
| 2084 | 23882 | 278 | AKF88466.1 | 18526 | 23883 | 303 | AMQ08961.1 | 18527 | 23884 | 169 | | | | |
| 2085 | 23885 | 278 | KNK74746.1 | 18529 | 23886 | 303 | KNK74745.1 | 18530 | 23887 | 169 | | | | |
| 2086 | 23888 | 278 | KNW86451.1 | 18532 | 23889 | 303 | KNW86450.1 | 18533 | 23890 | 169 | | | | |
| 2087 | 23891 | 277 | ALO88018.1 | 18535 | 23892 | 303 | ALO88019.1 | 18536 | 23893 | 169 | | | | |
| 2088 | 23894 | 278 | KTQ22862.1 | 18538 | 23895 | 287 | KTQ21994.1 | 18539 | 23896 | 169 | | | | |
| 2089 | 23897 | 278 | KUB19504.1 | 18541 | 23898 | 287 | KUB19292.1 | 18542 | 23899 | 169 | | | | |
| 2090 | 23900 | 278 | KTY61531.1 | 18544 | 23901 | 287 | KTY61368.1 | 18545 | 23902 | 169 | | | | |
| 2091 | 23903 | 278 | KUB06355.1 | 18547 | 23904 | 287 | KUB06250.1 | 18548 | 23905 | 169 | | | | |
| 2092 | 23906 | 278 | KUD95102.1 | 18550 | 23907 | 287 | KUD94943.1 | 18551 | 23908 | 169 | | | | |
| 2093 | 23909 | 278 | GAR53163.1 | 18553 | 23910 | 303 | GAR53164.1 | 18554 | 23911 | 169 | | | | |
| 2094 | 23912 | 282 | EDS74763.1 | 18556 | 23913 | 284 | EDS74762.1 | 18557 | 23914 | 217 | | | | |
| 2095 | 23915 | 291 | KUG13795.1 | 18559 | 23916 | 284 | KUG13797.1 | 18560 | 23917 | 168 | | | | |
| 2096 | 23918 | 278 | EIY23904.1 | 18562 | 23919 | 176 | EIY23906.1 | 18563 | 23920 | 176 | | | | |
| 2097 | 23921 | 297 | EOS20512.1 | 18565 | 23922 | 280 | EOS20510.1 | 18566 | 23923 | 154 | | | | |
| 2098 | 23924 | 278 | EEH86955.2 | 18568 | 23925 | 307 | EEH86956.1 | 18569 | 23926 | 169 | | | | |
| 2099 | 23927 | 293 | CDE34945.1 | 18571 | 23928 | 266 | CDE34947.1 | 18572 | 23929 | 185 | | | | |
| 2100 | 23930 | 278 | AKW15255.1 | 18574 | 23931 | 303 | AKW15260.1 | 18575 | 23932 | 291 | AKW15254.1 | 18576 | 23933 | 169 |

(Part C)

| | | | | AB | | | | AF | | | |
|---|---|---|---|---|---|---|---|---|---|---|---|
| | Y | Z | AA | ThsB (iv) | AC | AD | AE | ThsB (v) | AG | AH | |
| | ThsB (iv) | PRT SEQ | DNA SEQ | protein | ThsB (v) | PRT SEQ | DNA SEQ | protein | Genomic | Genomic | AI |
| 1 | protein_accession | ID NO: | ID NO: | length (aa) | protein_accession | ID NO: | ID NO: | length (aa) | Start Point | End Point | comments |
| 2 | | | | | | | | | 18199 | 21006 | |
| 3 | | | | | | | | | 629 | 2777 | |
| 4 | | | | | | | | | 5626 | 8433 | |
| 5 | | | | | | | | | 22562 | 25369 | |
| 6 | | | | | | | | | 51158 | 53041 | |
| 7 | | | | | | | | | 972 | 2894 | |
| 8 | | | | | | | | | 7781 | 10368 | |
| 9 | | | | | | | | | 4395990 | 4398131 | |
| 10 | | | | | | | | | 72122 | 74690 | |
| 11 | | | | | | | | | 36696 | 39253 | |
| 12 | | | | | | | | | 36738 | 39295 | |
| 13 | | | | | | | | | 12931 | 15738 | |
| 14 | | | | | | | | | 26929 | 28839 | |
| 15 | | | | | | | | | 168640 | 171278 | Additional ThsA homolog EGV07143.1 |
| 16 | | | | | | | | | 117544 | 120507 | |
| 17 | | | | | | | | | 6436 | 9650 | Additional ThsA homolog EHM75397.1 |
| 18 | | | | | | | | | 29271 | 31828 | |
| 19 | | | | | | | | | 13966 | 17199 | |
| 20 | | | | | | | | | 5865 | 9078 | |
| 21 | | | | | | | | | 89212 | 92444 | Additional ThsA homolog EIA14316.1 |
| 22 | | | | | | | | | 52056 | 55538 | Additional ThsA homolog EIC95445.1 and EIC95416.1 |
| 23 | | | | | | | | | 1081676 | 1083596 | |
| 24 | | | | | | | | | 66368 | 68925 | |
| 25 | | | | | | | | | 66447 | 69004 | |
| 26 | | | | | | | | | 17507 | 20048 | |
| 27 | | | | | | | | | 86 | 1182 | |
| 28 | | | | | | | | | 11678 | 13703 | |
| 29 | | | | | | | | | 329879 | 331798 | |
| 30 | | | | | | | | | 5296579 | 5302199 | |
| 31 | | | | | | | | | 2271775 | 2274990 | |
| 32 | | | | | | | | | 5587 | 8095 | |
| 33 | | | | | | | | | 7411 | 9563 | |
| 34 | | | | | | | | | 7716 | 9868 | |
| 35 | | | | | | | | | 166 | 2264 | |
| 36 | | | | | | | | | 331145 | 333209 | |
| 37 | | | | | | | | | 416875 | 418939 | |
| 38 | | | | | | | | | 9843 | 12589 | |
| 39 | | | | | | | | | 137838 | 139780 | |
| 40 | | | | | | | | | 3230645 | 3232587 | |
| 41 | | | | | | | | | 214042 | 218633 | |
| 42 | BAQ74698.1 | 13325 | 18682 | 282 | | | | | 3398464 | 3402455 | |
| 43 | | | | | | | | | 222931 | 225520 | |
| 44 | | | | | | | | | 46820 | 51171 | |
| 45 | | | | | | | | | 29183 | 31097 | |
| 46 | | | | | | | | | 46863 | 51213 | |
| 47 | | | | | | | | | 6531 | 9746 | |
| 48 | | | | | | | | | 58762 | 61977 | |
| 49 | | | | | | | | | 29869 | 33084 | |
| 50 | | | | | | | | | 6530 | 9744 | Additional ThsA homolog EPZ03687.1 |
| 51 | | | | | | | | | 29869 | 33083 | Additional ThsA homolog EPZ11496.1 |
| 52 | | | | | | | | | 1186644 | 1187914 | |
| 53 | | | | | | | | | 4035 | 5954 | |
| 54 | | | | | | | | | 4032 | 5951 | |
| 55 | ETR95047.1 | 13360 | 18717 | 392 | ETR95035.1 | 13361 | 18718 | 258 | 57073 | 63348 | |
| 56 | | | | | | | | | 102852 | 105529 | |
| 57 | | | | | | | | | 68333 | 70275 | |
| 58 | | | | | | | | | 29849 | 31768 | |
| 59 | | | | | | | | | 10611 | 13921 | |
| 60 | | | | | | | | | 38796 | 40715 | |
| 61 | ERT19164.1 | 13378 | 18735 | 284 | | | | | 47778 | 51812 | |
| 62 | | | | | | | | | 13859 | 16441 | |
| 63 | | | | | | | | | 12425 | 15043 | |
| 64 | | | | | | | | | 4783 | 6845 | |
| 65 | | | | | | | | | 15034 | 16957 | |
| 66 | | | | | | | | | 140001 | 142539 | |
| 67 | | | | | | | | | 28373 | 30424 | |
| 68 | | | | | | | | | 1 | 1825 | |
| 69 | | | | | | | | | 64004 | 65911 | |
| 70 | | | | | | | | | 166 | 2021 | |
| 71 | | | | | | | | | 6080 | 8527 | |
| 72 | | | | | | | | | 515442 | 517994 | |
| 73 | | | | | | | | | 674 | 3536 | |
| 74 | | | | | | | | | 139546 | 141467 | |
| 75 | | | | | | | | | 41136 | 43033 | |
| 76 | | | | | | | | | 13018 | 14436 | |
| 77 | | | | | | | | | 4125512 | 4127653 | |
| 78 | | | | | | | | | 5228544 | 5231702 | |
| 79 | | | | | | | | | 5761473 | 5764631 | |
| 80 | | | | | | | | | 1756503 | 1758422 | |
| 81 | | | | | | | | | 257376 | 259446 | |
| 82 | | | | | | | | | 3144948 | 3147551 | |
| 83 | | | | | | | | | 416071 | 423425 | |
| 84 | | | | | | | | | 1137270 | 1142967 | |
| 85 | | | | | | | | | 3070609 | 3073154 | |
| 86 | | | | | | | | | 1196726 | 1201628 | |
| 87 | | | | | | | | | 1362278 | 1364836 | |
| 88 | | | | | | | | | 1106775 | 1110408 | |
| 89 | | | | | | | | | 6843 | 9373 | |
| 90 | ABR34956.1 | 13455 | 18812 | 397 | | | | | 3262303 | 3271086 | |
| 91 | | | | | | | | | 193025 | 201754 | |
| 92 | ACA73841.1 | 13462 | 18819 | 284 | | | | | 3700507 | 3704541 | |
| 93 | | | | | | | | | 1522479 | 1525210 | |
| 94 | | | | | | | | | 159043 | 161228 | |
| 95 | | | | | | | | | 26150 | 28218 | |
| 96 | | | | | | | | | 3660129 | 3669571 | |
| 97 | | | | | | | | | 30739 | 34915 | |
| 98 | | | | | | | | | 8582744 | 8587380 | |
| 99 | | | | | | | | | 1144241 | 1146856 | |
| 100 | AEA61153.1 | 13487 | 18844 | 185 | AEA61148.1 | 13488 | 18845 | 380 | 2903631 | 2911620 | |
| 101 | | | | | | | | | 2114555 | 2117787 | Additional ThsA homolog AFH70386.1 |
| 102 | | | | | | | | | 463662 | 466219 | |
| 103 | | | | | | | | | 4207854 | 4209995 | |
| 104 | | | | | | | | | 2480783 | 2484341 | |
| 105 | | | | | | | | | 3773861 | 3775803 | |
| 106 | | | | | | | | | 2704998 | 2706901 | |
| 107 | | | | | | | | | 587283 | 590090 | |
| 108 | | | | | | | | | 3506208 | 3510086 | |
| 109 | | | | | | | | | 2181958 | 2185173 | |
| 110 | | | | | | | | | 5998143 | 6005265 | |
| 111 | | | | | | | | | 3932979 | 3934921 | |
| 112 | | | | | | | | | 1794531 | 1800432 | Additional ThsA homolog AHZ68659.1 |
| 113 | | | | | | | | | 3384959 | 3385740 | |
| 114 | | | | | | | | | 10892 | 12978 | |
| 115 | | | | | | | | | 3875996 | 3877938 | |
| 116 | | | | | | | | | 1315665 | 1318348 | |
| 117 | AIU01467.1 | 13536 | 18893 | 397 | | | | | 3262298 | 3271081 | |
| 118 | | | | | | | | | 4055767 | 4058527 | |
| 119 | | | | | | | | | 77843 | 79615 | Additional ThsA homolog AJK51070.1 |
| 120 | | | | | | | | | 333850 | 336261 | |
| 121 | | | | | | | | | 329400 | 331811 | |
| 122 | | | | | | | | | 347430 | 349849 | |
| 123 | | | | | | | | | 347423 | 349980 | |
| 124 | | | | | | | | | 3945969 | 3949326 | |
| 125 | | | | | | | | | 4205818 | 4210073 | |
| 126 | | | | | | | | | 1170644 | 1172700 | |
| 127 | | | | | | | | | 2330122 | 2333479 | |
| 128 | | | | | | | | | 1113818 | 1116217 | |
| 129 | | | | | | | | | 882751 | 885296 | |
| 130 | | | | | | | | | 2831462 | 2835547 | |
| 131 | | | | | | | | | 311495 | 313453 | |
| 132 | | | | | | | | | 6039155 | 6041758 | |
| 133 | | | | | | | | | 79256 | 82956 | |
| 134 | | | | | | | | | 1727530 | 1732447 | |
| 135 | | | | | | | | | 1268306 | 1270321 | |
| 136 | | | | | | | | | 3178713 | 3180655 | |
| 137 | | | | | | | | | 2230390 | 2233379 | |
| 138 | | | | | | | | | 2056187 | 2058744 | |
| 139 | | | | | | | | | 1551820 | 1554323 | |
| 140 | | | | | | | | | 1705326 | 1706706 | |
| 141 | | | | | | | | | 2176463 | 2179695 | |
| 142 | | | | | | | | | 2168129 | 2171362 | |
| 143 | | | | | | | | | 2280993 | 2284226 | |
| 144 | | | | | | | | | 3915939 | 3920392 | |
| 145 | | | | | | | | | 1704217 | 1705597 | |
| 146 | | | | | | | | | 2183524 | 2186331 | |
| 147 | | | | | | | | | 1361260 | 1364863 | Additional ThsA homolog AMJ40915.1 |
| 148 | | | | | | | | | 1163503 | 1166716 | |
| 149 | | | | | | | | | 5251680 | 5253446 | |
| 150 | | | | | | | | | 4624 | 9364 | |
| 151 | | | | | | | | | 123406 | 125800 | |
| 152 | | | | | | | | | 414 | 2808 | |
| 153 | | | | | | | | | 4336 | 6730 | |
| 154 | | | | | | | | | 112666 | 114575 | |
| 155 | CQJ38345.1 | 13644 | 19001 | 395 | | | | | 119502 | 126440 | |
| 156 | | | | | | | | | 170171 | 175125 | |
| 157 | | | | | | | | | 22084 | 24292 | |
| 158 | | | | | | | | | 15713 | 17632 | |
| 159 | | | | | | | | | 97 | 2016 | |
| 160 | | | | | | | | | 3516 | 5435 | |
| 161 | | | | | | | | | 86 | 2005 | |
| 162 | | | | | | | | | 126 | 2045 | |
| 163 | | | | | | | | | 86 | 2005 | |
| 164 | | | | | | | | | 86504 | 88423 | |
| 165 | | | | | | | | | 126 | 2045 | |
| 166 | | | | | | | | | 3572 | 5491 | |
| 167 | | | | | | | | | 86 | 2005 | |
| 168 | | | | | | | | | 86 | 2005 | |
| 169 | | | | | | | | | 86 | 2005 | |
| 170 | | | | | | | | | 3523 | 5442 | |
| 171 | | | | | | | | | 86 | 2005 | |
| 172 | | | | | | | | | 15712 | 17631 | |
| 173 | | | | | | | | | 15714 | 17633 | |
| 174 | | | | | | | | | 15715 | 17634 | |
| 175 | | | | | | | | | 15715 | 17634 | |
| 176 | | | | | | | | | 17014 | 18933 | |
| 177 | | | | | | | | | 86 | 2005 | |
| 178 | | | | | | | | | 15715 | 17634 | |
| 179 | | | | | | | | | 16634 | 18553 | |
| 180 | | | | | | | | | 17014 | 18933 | |
| 181 | | | | | | | | | 15714 | 17633 | |
| 182 | | | | | | | | | 86 | 2005 | |
| 183 | | | | | | | | | 86 | 2005 | |
| 184 | | | | | | | | | 86 | 2005 | |
| 185 | | | | | | | | | 15916 | 17835 | |
| 186 | | | | | | | | | 86 | 2005 | |
| 187 | | | | | | | | | 86 | 2005 | |
| 188 | | | | | | | | | 86 | 2005 | |
| 189 | | | | | | | | | 86 | 2005 | |
| 190 | | | | | | | | | 17014 | 18933 | |
| 191 | | | | | | | | | 16688 | 18607 | |
| 192 | | | | | | | | | 86 | 2005 | |
| 193 | | | | | | | | | 86 | 2005 | |
| 194 | | | | | | | | | 86 | 2005 | |
| 195 | | | | | | | | | 15714 | 17633 | |
| 196 | | | | | | | | | 86 | 2005 | |
| 197 | | | | | | | | | 15712 | 17631 | |
| 198 | | | | | | | | | 15707 | 17626 | |
| 199 | | | | | | | | | 15715 | 17634 | |
| 200 | | | | | | | | | 16688 | 18607 | |
| 201 | | | | | | | | | 86 | 2005 | |
| 202 | | | | | | | | | 15713 | 17632 | |
| 203 | | | | | | | | | 86 | 2005 | |
| 204 | | | | | | | | | 86 | 2005 | |
| 205 | | | | | | | | | 86 | 2005 | |
| 206 | | | | | | | | | 15727 | 17646 | |
| 207 | | | | | | | | | 86 | 2005 | |
| 208 | | | | | | | | | 19604 | 21523 | |
| 209 | | | | | | | | | 15675 | 17594 | |
| 210 | | | | | | | | | 86 | 2005 | |
| 211 | | | | | | | | | 86 | 2005 | |
| 212 | | | | | | | | | 103 | 2022 | |
| 213 | | | | | | | | | 86 | 2005 | |
| 214 | | | | | | | | | 15667 | 17586 | |
| 215 | | | | | | | | | 86 | 2005 | |
| 216 | | | | | | | | | 86 | 2005 | |
| 217 | | | | | | | | | 86 | 2005 | |
| 218 | | | | | | | | | 86 | 2005 | |
| 219 | | | | | | | | | 111 | 2030 | |
| 220 | | | | | | | | | 86 | 2005 | |
| 221 | | | | | | | | | 86 | 2005 | |
| 222 | | | | | | | | | 86 | 2005 | |
| 223 | | | | | | | | | 15714 | 17633 | |
| 224 | | | | | | | | | 20763 | 22682 | |
| 225 | | | | | | | | | 15715 | 17634 | |
| 226 | | | | | | | | | 90 | 2009 | |
| 227 | | | | | | | | | 86 | 2005 | |
| 228 | | | | | | | | | 86 | 2005 | |
| 229 | | | | | | | | | 86 | 2005 | |
| 230 | | | | | | | | | 15715 | 17634 | |
| 231 | | | | | | | | | 86 | 2005 | |
| 232 | | | | | | | | | 90 | 2009 | |
| 233 | | | | | | | | | 126 | 2045 | |
| 234 | | | | | | | | | 93 | 2012 | |
| 235 | | | | | | | | | 86 | 2005 | |
| 236 | | | | | | | | | 86 | 2005 | |
| 237 | | | | | | | | | 15713 | 17632 | |
| 238 | | | | | | | | | 86 | 2005 | |
| 239 | | | | | | | | | 15711 | 17630 | |
| 240 | | | | | | | | | 15713 | 17632 | |
| 241 | | | | | | | | | 86 | 2005 | |
| 242 | | | | | | | | | 9021 | 10940 | |
| 243 | | | | | | | | | 86 | 2005 | |
| 244 | | | | | | | | | 86 | 2005 | |
| 245 | | | | | | | | | 86 | 2005 | |
| 246 | | | | | | | | | 85 | 2004 | |
| 247 | | | | | | | | | 86 | 2005 | |
| 248 | | | | | | | | | 86 | 2005 | |
| 249 | | | | | | | | | 19605 | 21524 | |
| 250 | | | | | | | | | 15916 | 17835 | |
| 251 | | | | | | | | | 15715 | 17634 | |
| 252 | | | | | | | | | 15916 | 17835 | |
| 253 | | | | | | | | | 15916 | 17835 | |
| 254 | | | | | | | | | 86 | 2005 | |
| 255 | | | | | | | | | 85 | 2004 | |
| 256 | | | | | | | | | 86 | 2005 | |
| 257 | | | | | | | | | 86 | 2005 | |
| 258 | | | | | | | | | 86 | 2005 | |
| 259 | | | | | | | | | 3523 | 5442 | |
| 260 | | | | | | | | | 15715 | 17634 | |
| 261 | | | | | | | | | 15715 | 17634 | |
| 262 | | | | | | | | | 15715 | 17634 | |
| 263 | | | | | | | | | 86 | 2005 | |
| 264 | | | | | | | | | 86 | 2005 | |
| 265 | | | | | | | | | 86 | 2005 | |
| 266 | | | | | | | | | 86 | 2005 | |
| 267 | | | | | | | | | 86 | 2005 | |
| 268 | | | | | | | | | 15916 | 17835 | |
| 269 | | | | | | | | | 86 | 2005 | |
| 270 | | | | | | | | | 86 | 2005 | |
| 271 | | | | | | | | | 86 | 2005 | |
| 272 | | | | | | | | | 15916 | 17835 | |
| 273 | | | | | | | | | 15916 | 17835 | |
| 274 | | | | | | | | | 15916 | 17835 | |
| 275 | | | | | | | | | 15713 | 17632 | |
| 276 | | | | | | | | | 15715 | 17634 | |
| 277 | | | | | | | | | 85 | 2004 | |
| 278 | | | | | | | | | 20706 | 22625 | |
| 279 | | | | | | | | | 85 | 2004 | |
| 280 | | | | | | | | | 85 | 2004 | |
| 281 | | | | | | | | | 86 | 2005 | |
| 282 | | | | | | | | | 86 | 2005 | |
| 283 | | | | | | | | | 90 | 2009 | |
| 284 | | | | | | | | | 86 | 2005 | |
| 285 | | | | | | | | | 15714 | 17633 | |
| 286 | | | | | | | | | 15719 | 17638 | |
| 287 | | | | | | | | | 15710 | 17629 | |
| 288 | | | | | | | | | 15726 | 17645 | |
| 289 | | | | | | | | | 3523 | 5442 | |
| 290 | | | | | | | | | 15715 | 17634 | |
| 291 | | | | | | | | | 15915 | 17834 | |
| 292 | | | | | | | | | 86 | 2005 | |
| 293 | | | | | | | | | 86 | 2005 | |
| 294 | | | | | | | | | 86 | 2005 | |
| 295 | | | | | | | | | 86 | 2005 | |
| 296 | | | | | | | | | 86 | 2005 | |
| 297 | | | | | | | | | 86 | 2005 | |
| 298 | | | | | | | | | 15715 | 17634 | |
| 299 | | | | | | | | | 86 | 2005 | |
| 300 | | | | | | | | | 86 | 2005 | |
| 301 | | | | | | | | | 86 | 2005 | |
| 302 | | | | | | | | | 86 | 2005 | |
| 303 | | | | | | | | | 86 | 2005 | |
| 304 | | | | | | | | | 86 | 2005 | |
| 305 | | | | | | | | | 86 | 2005 | |
| 306 | | | | | | | | | 86 | 2005 | |
| 307 | | | | | | | | | 86 | 2005 | |
| 308 | | | | | | | | | 86 | 2005 | |
| 309 | | | | | | | | | 86 | 2005 | |
| 310 | | | | | | | | | 3520 | 5439 | |
| 311 | | | | | | | | | 86 | 2005 | |
| 312 | | | | | | | | | 86 | 2005 | |
| 313 | | | | | | | | | 86 | 2005 | |
| 314 | | | | | | | | | 86 | 2005 | |
| 315 | | | | | | | | | 15715 | 17634 | |
| 316 | | | | | | | | | 85 | 2004 | |
| 317 | | | | | | | | | 19612 | 21531 | |
| 318 | | | | | | | | | 15715 | 17634 | |
| 319 | | | | | | | | | 15712 | 17631 | |
| 320 | | | | | | | | | 15715 | 17634 | |
| 321 | | | | | | | | | 19605 | 21524 | |
| 322 | | | | | | | | | 86 | 2005 | |
| 323 | | | | | | | | | 15714 | 17633 | |
| 324 | | | | | | | | | 15715 | 17634 | |
| 325 | | | | | | | | | 85 | 2004 | |
| 326 | | | | | | | | | 15715 | 17634 | |
| 327 | | | | | | | | | 19605 | 21524 | |
| 328 | | | | | | | | | 85 | 2004 | |
| 329 | | | | | | | | | 86 | 2005 | |
| 330 | | | | | | | | | 86 | 2005 | |
| 331 | | | | | | | | | 15714 | 17633 | |
| 332 | | | | | | | | | 15713 | 17632 | |
| 333 | | | | | | | | | 3523 | 5442 | |
| 334 | | | | | | | | | 15715 | 17634 | |
| 335 | | | | | | | | | 19605 | 21524 | |
| 336 | | | | | | | | | 86 | 2005 | |
| 337 | | | | | | | | | 86 | 2005 | |
| 338 | | | | | | | | | 57301 | 59220 | |
| 339 | | | | | | | | | 16030 | 17949 | |
| 340 | | | | | | | | | 86 | 2005 | |
| 341 | | | | | | | | | 86 | 2005 | |
| 342 | | | | | | | | | 86 | 2005 | |
| 343 | | | | | | | | | 15714 | 17633 | |
| 344 | | | | | | | | | 15715 | 17634 | |
| 345 | | | | | | | | | 86 | 2005 | |
| 346 | | | | | | | | | 90 | 2009 | |
| 347 | | | | | | | | | 85 | 2004 | |
| 348 | | | | | | | | | 86 | 2005 | |
| 349 | | | | | | | | | 21322 | 23241 | |
| 350 | | | | | | | | | 86 | 2005 | |
| 351 | | | | | | | | | 86 | 2005 | |
| 352 | | | | | | | | | 86 | 2005 | |
| 353 | | | | | | | | | 86 | 2005 | |
| 354 | | | | | | | | | 86 | 2005 | |
| 355 | | | | | | | | | 84 | 2003 | |
| 356 | | | | | | | | | 86 | 2005 | |
| 357 | | | | | | | | | 86 | 2005 | |
| 358 | | | | | | | | | 86 | 2005 | |
| 359 | | | | | | | | | 86 | 2005 | |
| 360 | | | | | | | | | 57 | 1976 | |
| 361 | | | | | | | | | 86 | 2005 | |
| 362 | | | | | | | | | 90 | 2009 | |
| 363 | | | | | | | | | 86 | 2005 | |
| 364 | | | | | | | | | 126 | 2045 | |
| 365 | | | | | | | | | 90 | 2009 | |
| 366 | | | | | | | | | 86 | 2005 | |
| 367 | | | | | | | | | 86 | 2005 | |
| 368 | | | | | | | | | 86 | 2005 | |
| 369 | | | | | | | | | 3523 | 5442 | |
| 370 | | | | | | | | | 86 | 2005 | |
| 371 | | | | | | | | | 86 | 2005 | |
| 372 | | | | | | | | | 86 | 2005 | |
| 373 | | | | | | | | | 86 | 2005 | |
| 374 | | | | | | | | | 86 | 2005 | |
| 375 | | | | | | | | | 86 | 2005 | |
| 376 | | | | | | | | | 86 | 2005 | |
| 377 | | | | | | | | | 3523 | 5442 | |
| 378 | | | | | | | | | 3523 | 5442 | |
| 379 | | | | | | | | | 86 | 2005 | |
| 380 | | | | | | | | | 86 | 2005 | |
| 381 | | | | | | | | | 86 | 2005 | |
| 382 | | | | | | | | | 20582 | 22497 | |
| 383 | | | | | | | | | 69792 | 71707 | |
| 384 | | | | | | | | | 473455 | 475428 | |
| 385 | CUI40923.1 | 14109 | 19466 | 380 | | | | | 61503 | 69491 | |
| 386 | | | | | | | | | 29199 | 31433 | |
| 387 | | | | | | | | | 91096 | 93166 | |
| 388 | | | | | | | | | 13294 | 15364 | |
| 389 | | | | | | | | | 91096 | 93166 | |
| 390 | | | | | | | | | 208463 | 212572 | |
| 391 | | | | | | | | | 52908 | 54850 | |
| 392 | | | | | | | | | 205694 | 207636 | |
| 393 | | | | | | | | | 51817 | 53759 | |
| 394 | | | | | | | | | 52565 | 54507 | |
| 395 | | | | | | | | | 40795 | 42737 | |
| 396 | | | | | | | | | 208781 | 210723 | |
| 397 | | | | | | | | | 416502 | 418444 | |
| 398 | | | | | | | | | 52868 | 54810 | |
| 399 | | | | | | | | | 205143 | 207085 | |
| 400 | | | | | | | | | 205136 | 207078 | |
| 401 | | | | | | | | | 52762 | 54704 | |
| 402 | | | | | | | | | 48586 | 50528 | |
| 403 | | | | | | | | | 205291 | 207233 | |
| 404 | | | | | | | | | 227349 | 229291 | |
| 405 | | | | | | | | | 54635 | 56577 | |
| 406 | | | | | | | | | 205150 | 207092 | |
| 407 | | | | | | | | | 52282 | 54224 | |
| 408 | | | | | | | | | 98754 | 100696 | |
| 409 | | | | | | | | | 459698 | 461640 | |
| 410 | | | | | | | | | 1072447 | 1074389 | |
| 411 | | | | | | | | | 52866 | 54808 | |
| 412 | | | | | | | | | 52771 | 54713 | |
| 413 | | | | | | | | | 53129 | 55071 | |
| 414 | | | | | | | | | 52821 | 54763 | |
| 415 | | | | | | | | | 381639 | 383581 | |
| 416 | | | | | | | | | 251604 | 253547 | Additional ThsA homolog CUZ65536.1 |
| 417 | | | | | | | | | 227335 | 229277 | |
| 418 | | | | | | | | | 215912 | 217854 | |
| 419 | | | | | | | | | 170621 | 172563 | |
| 420 | | | | | | | | | 54612 | 56554 | |
| 421 | | | | | | | | | 259463 | 261405 | |
| 422 | | | | | | | | | 52769 | 54711 | |
| 423 | | | | | | | | | 215028 | 216970 | |
| 424 | | | | | | | | | 55119 | 57061 | |
| 425 | | | | | | | | | 145344 | 147286 | |
| 426 | | | | | | | | | 51538 | 53480 | |
| 427 | | | | | | | | | 54675 | 56617 | |
| 428 | | | | | | | | | 52756 | 54698 | |
| 429 | | | | | | | | | 247802 | 249745 | Additional ThsA homolog CUZ82852.1 |
| 430 | | | | | | | | | 468965 | 470907 | |
| 431 | | | | | | | | | 52873 | 54815 | |
| 432 | | | | | | | | | 58418 | 60361 | Additional ThsA homolog CUZ43256.1 |
| 433 | | | | | | | | | 52608 | 54550 | |
| 434 | | | | | | | | | 98756 | 100698 | |
| 435 | | | | | | | | | 51815 | 53757 | |
| 436 | | | | | | | | | 48717 | 50659 | |
| 437 | | | | | | | | | 378607 | 380549 | |
| 438 | | | | | | | | | 259489 | 261431 | |
| 439 | | | | | | | | | 259461 | 261403 | |
| 440 | | | | | | | | | 53320 | 55262 | |
| 441 | | | | | | | | | 52797 | 54739 | |
| 442 | | | | | | | | | 259463 | 261405 | |
| 443 | | | | | | | | | 71559 | 73501 | |
| 444 | | | | | | | | | 71562 | 73504 | |
| 445 | | | | | | | | | 205150 | 207092 | |
| 446 | | | | | | | | | 54661 | 56603 | |
| 447 | | | | | | | | | 2774448 | 2776391 | Additional ThsA homolog CVB24107.1 |
| 448 | | | | | | | | | 474999 | 476941 | |
| 449 | | | | | | | | | 205287 | 207229 | |
| 450 | | | | | | | | | 54153 | 56096 | Additional ThsA homolog CVB73330.1 |
| 451 | | | | | | | | | 287703 | 289646 | Additional ThsA homolog CVA22598.1 |
| 452 | | | | | | | | | 205297 | 207239 | |
| 453 | | | | | | | | | 54674 | 56616 | |
| 454 | | | | | | | | | 227325 | 229267 | |
| 455 | | | | | | | | | 259463 | 261405 | |
| 456 | | | | | | | | | 52811 | 54753 | |
| 457 | | | | | | | | | 259443 | 261385 | |
| 458 | | | | | | | | | 259454 | 261396 | |
| 459 | | | | | | | | | 52807 | 54749 | |
| 460 | | | | | | | | | 54610 | 56552 | |
| 461 | | | | | | | | | 54003 | 55945 | |
| 462 | | | | | | | | | 52860 | 54802 | |
| 463 | | | | | | | | | 555244 | 557186 | |
| 464 | | | | | | | | | 205101 | 207043 | |
| 465 | | | | | | | | | 259463 | 261405 | |
| 466 | | | | | | | | | 55632 | 57574 | |
| 467 | | | | | | | | | 469398 | 471341 | Additional ThsA homolog CVB85980.1 |
| 468 | | | | | | | | | 52988 | 54930 | |
| 469 | | | | | | | | | 53025 | 54967 | |
| 470 | | | | | | | | | 52986 | 54928 | |
| 471 | | | | | | | | | 245145 | 247087 | |
| 472 | | | | | | | | | 228659 | 230601 | |
| 473 | | | | | | | | | 259502 | 261444 | |
| 474 | | | | | | | | | 72560 | 74502 | |
| 475 | | | | | | | | | 54144 | 56087 | Additional ThsA homolog CVD13385.1 |
| 476 | | | | | | | | | 53587 | 55529 | |
| 477 | | | | | | | | | 205145 | 207087 | |
| 478 | | | | | | | | | 259537 | 261479 | |
| 479 | | | | | | | | | 52763 | 54705 | |
| 480 | | | | | | | | | 204839 | 206781 | |
| 481 | | | | | | | | | 250690 | 252632 | |
| 482 | | | | | | | | | 52792 | 54734 | |
| 483 | | | | | | | | | 52806 | 54748 | |
| 484 | | | | | | | | | 259463 | 261405 | |
| 485 | | | | | | | | | 53587 | 55529 | |
| 486 | | | | | | | | | 54693 | 56635 | |
| 487 | | | | | | | | | 381593 | 383535 | |
| 488 | | | | | | | | | 259463 | 261405 | |
| 489 | | | | | | | | | 53129 | 55071 | |
| 490 | | | | | | | | | 229979 | 231922 | Additional ThsA homolog CVC59825.1 |
| 491 | | | | | | | | | 40797 | 42739 | |
| 492 | | | | | | | | | 53318 | 55260 | |
| 493 | | | | | | | | | 474325 | 476267 | |
| 494 | | | | | | | | | 276598 | 278540 | |
| 495 | | | | | | | | | 205145 | 207087 | |
| 496 | | | | | | | | | 56080 | 58023 | Additional ThsA homolog CVD28877.1 |
| 497 | | | | | | | | | 203403 | 205345 | |
| 498 | | | | | | | | | 205188 | 207130 | |
| 499 | | | | | | | | | 100253 | 102196 | Additional ThsA homolog CVC40444.1 |
| 500 | | | | | | | | | 54152 | 56095 | Additional ThsA homolog CVE27748.1 |
| 501 | | | | | | | | | 52837 | 54779 | |
| 502 | | | | | | | | | 287741 | 289684 | Additional ThsA homolog CVE69273.1 |
| 503 | | | | | | | | | 250718 | 252660 | |
| 504 | | | | | | | | | 53129 | 55071 | |
| 505 | | | | | | | | | 409590 | 411532 | |
| 506 | | | | | | | | | 225217 | 227159 | |
| 507 | | | | | | | | | 55828 | 57770 | |
| 508 | | | | | | | | | 52615 | 54557 | |
| 509 | | | | | | | | | 259463 | 261405 | |
| 510 | | | | | | | | | 85596 | 87538 | |
| 511 | | | | | | | | | 40786 | 42728 | |
| 512 | | | | | | | | | 51813 | 53755 | |
| 513 | | | | | | | | | 331093 | 333036 | Additional ThsA homolog CVE63008.1 |
| 514 | | | | | | | | | 205143 | 207085 | |
| 515 | | | | | | | | | 246483 | 248425 | |
| 516 | | | | | | | | | 55632 | 57574 | |
| 517 | | | | | | | | | 203360 | 205302 | |
| 518 | | | | | | | | | 203361 | 205303 | |
| 519 | | | | | | | | | 53129 | 55071 | |
| 520 | | | | | | | | | 302434 | 304376 | |
| 521 | | | | | | | | | 205143 | 207085 | |
| 522 | | | | | | | | | 250747 | 252689 | |
| 523 | | | | | | | | | 51815 | 53757 | |
| 524 | | | | | | | | | 52610 | 54552 | |
| 525 | | | | | | | | | 218394 | 220336 | |
| 526 | | | | | | | | | 204804 | 206746 | |
| 527 | | | | | | | | | 56080 | 58023 | Additional ThsA homolog CVF09827.1 |
| 528 | | | | | | | | | 225215 | 227157 | |
| 529 | | | | | | | | | 380827 | 382769 | |
| 530 | | | | | | | | | 507350 | 509292 | |
| 531 | | | | | | | | | 205145 | 207087 | |
| 532 | | | | | | | | | 205180 | 207122 | |
| 533 | | | | | | | | | 468279 | 470221 | |
| 534 | | | | | | | | | 204811 | 206753 | |
| 535 | | | | | | | | | 2911354 | 2913296 | |
| 536 | | | | | | | | | 204806 | 206748 | |
| 537 | | | | | | | | | 48895 | 50837 | |
| 538 | | | | | | | | | 250731 | 252673 | |
| 539 | | | | | | | | | 250678 | 252620 | |
| 540 | | | | | | | | | 209597 | 211539 | |
| 541 | | | | | | | | | 205292 | 207234 | |
| 542 | | | | | | | | | 218370 | 220312 | |
| 543 | | | | | | | | | 204799 | 206741 | |
| 544 | | | | | | | | | 259507 | 261449 | |
| 545 | | | | | | | | | 98756 | 100698 | |
| 546 | | | | | | | | | 52831 | 54773 | |
| 547 | | | | | | | | | 229166 | 231108 | |
| 548 | | | | | | | | | 229192 | 231134 | |
| 549 | | | | | | | | | 52862 | 54804 | |
| 550 | | | | | | | | | 469390 | 471333 | Additional ThsA homolog CVF82665.1 |
| 551 | | | | | | | | | 205176 | 207118 | |
| 552 | | | | | | | | | 86294 | 88236 | |
| 553 | | | | | | | | | 195844 | 197787 | Additional ThsA homolog CVG82395.1 |
| 554 | | | | | | | | | 259485 | 261427 | |
| 555 | | | | | | | | | 52838 | 54780 | |
| 556 | | | | | | | | | 259463 | 261405 | |
| 557 | | | | | | | | | 205143 | 207085 | |
| 558 | | | | | | | | | 204848 | 206790 | |
| 559 | | | | | | | | | 52908 | 54850 | |
| 560 | | | | | | | | | 48895 | 50837 | |
| 561 | | | | | | | | | 204800 | 206742 | |
| 562 | | | | | | | | | 205105 | 207047 | |
| 563 | | | | | | | | | 52761 | 54703 | |
| 564 | | | | | | | | | 205143 | 207085 | |
| 565 | | | | | | | | | 804248 | 806190 | |
| 566 | | | | | | | | | 42050 | 43992 | |
| 567 | | | | | | | | | 259463 | 261405 | |
| 568 | | | | | | | | | 98758 | 100700 | |
| 569 | | | | | | | | | 205139 | 207081 | |
| 570 | | | | | | | | | 203358 | 205300 | |
| 571 | | | | | | | | | 245017 | 246902 | |
| 572 | | | | | | | | | 244655 | 246597 | |
| 573 | | | | | | | | | 204808 | 206750 | |
| 574 | | | | | | | | | 55294 | 57236 | |
| 575 | | | | | | | | | 40785 | 42727 | |
| 576 | | | | | | | | | 146358 | 148300 | |
| 577 | | | | | | | | | 203356 | 205298 | |
| 578 | | | | | | | | | 213928 | 215870 | |
| 579 | | | | | | | | | 53145 | 55087 | |
| 580 | | | | | | | | | 302444 | 304386 | |
| 581 | | | | | | | | | 205171 | 207113 | |
| 582 | | | | | | | | | 86298 | 88240 | |
| 583 | | | | | | | | | 225285 | 227227 | |
| 584 | | | | | | | | | 2489848 | 2491790 | |
| 585 | | | | | | | | | 48902 | 50844 | |
| 586 | | | | | | | | | 205139 | 207081 | |
| 587 | | | | | | | | | 52816 | 54758 | |
| 588 | | | | | | | | | 48889 | 50831 | |
| 589 | | | | | | | | | 259463 | 261405 | |
| 590 | | | | | | | | | 276608 | 278550 | |
| 591 | | | | | | | | | 52823 | 54765 | |
| 592 | | | | | | | | | 219855 | 224432 | |
| 593 | | | | | | | | | 665752 | 668658 | |
| 594 | | | | | | | | | 10795 | 15202 | Additional ThsA homolog CVJ34926.1 |
| 595 | | | | | | | | | 91413 | 93483 | |
| 596 | | | | | | | | | 91412 | 93482 | |
| 597 | | | | | | | | | 145450 | 148647 | |
| 598 | | | | | | | | | 170139 | 172016 | |
| 599 | | | | | | | | | 147107 | 150314 | |
| 600 | | | | | | | | | 5221 | 8428 | |
| 601 | | | | | | | | | 27175 | 28106 | |
| 602 | | | | | | | | | 89207 | 90138 | |
| 603 | | | | | | | | | 56578 | 57509 | |
| 604 | | | | | | | | | 2764 | 3695 | |
| 605 | | | | | | | | | 755402 | 757321 | |
| 606 | | | | | | | | | 2281482 | 2283690 | Additional ThsA homolog CBK94115.1 |
| 607 | | | | | | | | | 2147940 | 2151155 | |
| 608 | | | | | | | | | 5550 | 8081 | |
| 609 | | | | | | | | | 216318 | 218740 | |
| 610 | | | | | | | | | 381949 | 385460 | |
| 611 | | | | | | | | | 5760 | 7808 | |
| 612 | | | | | | | | | 300284 | 303091 | |
| 613 | | | | | | | | | 49142 | 51298 | |
| 614 | | | | | | | | | 1929426 | 1932118 | |
| 615 | | | | | | | | | 14797 | 17604 | |
| 616 | | | | | | | | | 684310 | 687507 | |
| 617 | | | | | | | | | 127257 | 129883 | |
| 618 | | | | | | | | | 2141168 | 2149732 | |
| 619 | | | | | | | | | 3041325 | 3043214 | |
| 620 | | | | | | | | | 3085346 | 3087231 | |
| 621 | | | | | | | | | 553 | 2600 | |
| 622 | | | | | | | | | 10886 | 13693 | |
| 623 | | | | | | | | | 14764 | 17571 | |
| 624 | | | | | | | | | 10882 | 13689 | |
| 625 | | | | | | | | | 34164 | 36837 | |
| 626 | | | | | | | | | 58272 | 62622 | |
| 627 | | | | | | | | | 7752 | 11008 | Additional ThsA homolog EWC94486.1 |
| 628 | | | | | | | | | 270 | 5901 | |
| 629 | | | | | | | | | 3054 | 11860 | |
| 630 | EXE88613.1 | 14621 | 19978 | 405 | EXE88615.1 | 14622 | 19979 | 261 | 264895 | 270729 | |
| 631 | | | | | | | | | 55048 | 58095 | |
| 632 | | | | | | | | | 95834 | 100666 | |
| 633 | | | | | | | | | 284960 | 286849 | |
| 634 | | | | | | | | | 509807 | 511734 | |
| 635 | | | | | | | | | 2108537 | 2115530 | |
| 636 | | | | | | | | | 708642 | 710850 | |
| 637 | | | | | | | | | 599550 | 602414 | |
| 638 | | | | | | | | | 83092 | 84822 | |
| 639 | | | | | | | | | 176458 | 181638 | |
| 640 | | | | | | | | | 177762 | 182743 | |
| 641 | | | | | | | | | 207 | 2134 | |
| 642 | | | | | | | | | 251026 | 252953 | |
| 643 | | | | | | | | | 4590 | 6517 | |
| 644 | | | | | | | | | 2641819 | 2643746 | |
| 645 | | | | | | | | | 442583 | 444510 | |
| 646 | | | | | | | | | 430783 | 432710 | |
| 647 | | | | | | | | | 251121 | 253048 | |
| 648 | | | | | | | | | 205694 | 207650 | |
| 649 | | | | | | | | | 4337287 | 4339430 | |
| 650 | | | | | | | | | 154987 | 158145 | |
| 651 | | | | | | | | | 19805 | 21921 | |
| 652 | | | | | | | | | 417 | 3971 | |
| 653 | | | | | | | | | 13781 | 16588 | |
| 654 | | | | | | | | | 7847 | 10654 | |
| 655 | | | | | | | | | 4608 | 7415 | |
| 656 | | | | | | | | | 10909 | 13716 | |
| 657 | | | | | | | | | 36794 | 39601 | |
| 658 | | | | | | | | | 10909 | 13716 | |
| 659 | | | | | | | | | 36880 | 39687 | |
| 660 | | | | | | | | | 42693 | 45500 | |
| 661 | | | | | | | | | 42768 | 45575 | |
| 662 | | | | | | | | | 42629 | 45436 | |
| 663 | | | | | | | | | 13781 | 16588 | |
| 664 | | | | | | | | | 2627330 | 2629968 | |
| 665 | | | | | | | | | 166982 | 169811 | |
| 666 | | | | | | | | | 1603 | 3518 | |
| 667 | | | | | | | | | 23156 | 26372 | |
| 668 | | | | | | | | | 58886 | 62101 | |
| 669 | | | | | | | | | 58887 | 62102 | |
| 670 | | | | | | | | | 58821 | 62036 | |
| 671 | | | | | | | | | 58821 | 62036 | |
| 672 | | | | | | | | | 58887 | 62102 | |
| 673 | | | | | | | | | 58887 | 62102 | |
| 674 | | | | | | | | | 2720 | 5935 | |
| 675 | | | | | | | | | 11491 | 14706 | |
| 676 | | | | | | | | | 58887 | 62102 | |
| 677 | | | | | | | | | 58887 | 61046 | |
| 678 | | | | | | | | | 58887 | 62102 | |
| 679 | | | | | | | | | 58887 | 62102 | |
| 680 | | | | | | | | | 3137 | 6352 | |
| 681 | | | | | | | | | 58887 | 62102 | |
| 682 | | | | | | | | | 58887 | 62102 | |
| 683 | | | | | | | | | 58887 | 62102 | |
| 684 | | | | | | | | | 58887 | 62102 | |
| 685 | | | | | | | | | 89236 | 92451 | |
| 686 | | | | | | | | | 58887 | 62102 | |
| 687 | | | | | | | | | 58881 | 62096 | |
| 688 | | | | | | | | | 58887 | 62102 | |
| 689 | | | | | | | | | 89217 | 92431 | Additional ThsA homolog EZR97100.1 |
| 690 | | | | | | | | | 58887 | 62102 | |
| 691 | | | | | | | | | 89217 | 92432 | |
| 692 | | | | | | | | | 58653 | 61868 | |
| 693 | | | | | | | | | 58887 | 62102 | |
| 694 | | | | | | | | | 11494 | 14709 | |
| 695 | | | | | | | | | 89253 | 92468 | |
| 696 | | | | | | | | | 17149 | 20364 | |
| 697 | | | | | | | | | 58886 | 62101 | |
| 698 | | | | | | | | | 89215 | 92430 | |
| 699 | | | | | | | | | 58887 | 62102 | |
| 700 | | | | | | | | | 58887 | 62102 | |
| 701 | | | | | | | | | 51382 | 54597 | |
| 702 | | | | | | | | | 58887 | 62102 | |
| 703 | | | | | | | | | 58887 | 62102 | |
| 704 | | | | | | | | | 58887 | 62102 | |
| 705 | | | | | | | | | 27394 | 30609 | |
| 706 | | | | | | | | | 11491 | 14706 | |
| 707 | | | | | | | | | 58887 | 62102 | |
| 708 | | | | | | | | | 89217 | 92432 | |
| 709 | | | | | | | | | 58887 | 62102 | |
| 710 | | | | | | | | | 89087 | 92302 | |
| 711 | | | | | | | | | 58821 | 62036 | |
| 712 | | | | | | | | | 58821 | 62036 | |
| 713 | | | | | | | | | 89168 | 92383 | |
| 714 | | | | | | | | | 102659 | 105874 | |
| 715 | | | | | | | | | 58887 | 62102 | |
| 716 | | | | | | | | | 56011 | 59226 | |
| 717 | | | | | | | | | 58887 | 62102 | |
| 718 | | | | | | | | | 58887 | 62102 | |
| 719 | | | | | | | | | 58887 | 62101 | |
| 720 | | | | | | | | | 58887 | 62102 | |
| 721 | | | | | | | | | 13914 | 17129 | |
| 722 | | | | | | | | | 58887 | 62102 | |
| 723 | | | | | | | | | 58887 | 62102 | |
| 724 | | | | | | | | | 58887 | 62102 | |
| 725 | | | | | | | | | 58887 | 62102 | |
| 726 | | | | | | | | | 58886 | 62101 | |
| 727 | | | | | | | | | 58887 | 62102 | |
| 728 | | | | | | | | | 58887 | 62102 | |
| 729 | | | | | | | | | 35404 | 38619 | |
| 730 | | | | | | | | | 58887 | 62102 | |
| 731 | | | | | | | | | 20381 | 23596 | |
| 732 | | | | | | | | | 58887 | 62102 | |
| 733 | | | | | | | | | 58887 | 62102 | |
| 734 | | | | | | | | | 58887 | 62102 | |
| 735 | | | | | | | | | 11491 | 14706 | |
| 736 | | | | | | | | | 86075 | 89290 | |
| 737 | | | | | | | | | 58887 | 62102 | |
| 738 | | | | | | | | | 58887 | 62101 | |
| 739 | | | | | | | | | 58887 | 62102 | |
| 740 | | | | | | | | | 58830 | 62045 | |
| 741 | | | | | | | | | 59025 | 62239 | Additional ThsA homolog EZT27374.1 |
| 742 | | | | | | | | | 58887 | 62102 | |
| 743 | | | | | | | | | 58887 | 62102 | |
| 744 | | | | | | | | | 58887 | 62102 | |
| 745 | | | | | | | | | 58821 | 62036 | |
| 746 | | | | | | | | | 89258 | 92473 | |
| 747 | | | | | | | | | 58692 | 61907 | |
| 748 | | | | | | | | | 58887 | 62101 | Additional ThsA homolog EZT47450.1 |
| 749 | | | | | | | | | 89211 | 92425 | Additional ThsA homolog EZT50535.1 |
| 750 | | | | | | | | | 58886 | 62101 | |
| 751 | | | | | | | | | 58887 | 62102 | |
| 752 | | | | | | | | | 58887 | 62102 | |
| 753 | | | | | | | | | 89173 | 92388 | |
| 754 | | | | | | | | | 85365 | 88580 | |
| 755 | | | | | | | | | 58887 | 62102 | |
| 756 | | | | | | | | | 58887 | 62102 | |
| 757 | | | | | | | | | 58896 | 62111 | |
| 758 | | | | | | | | | 58887 | 62102 | |
| 759 | | | | | | | | | 58887 | 62102 | |
| 760 | | | | | | | | | 58887 | 62102 | |
| 761 | | | | | | | | | 5927 | 9142 | |
| 762 | | | | | | | | | 58887 | 62102 | |
| 763 | | | | | | | | | 58887 | 62102 | |
| 764 | | | | | | | | | 58653 | 61868 | |
| 765 | | | | | | | | | 58821 | 62036 | |
| 766 | | | | | | | | | 58888 | 62103 | |
| 767 | | | | | | | | | 58887 | 62102 | |
| 768 | | | | | | | | | 58887 | 62102 | |
| 769 | | | | | | | | | 58887 | 62102 | |
| 770 | | | | | | | | | 58887 | 62102 | |
| 771 | | | | | | | | | 1635 | 3550 | |
| 772 | | | | | | | | | 1609 | 3524 | |
| 773 | | | | | | | | | 135764 | 138293 | |
| 774 | | | | | | | | | 1226749 | 1227659 | |
| 775 | | | | | | | | | 107019 | 109625 | |
| 776 | | | | | | | | | 92321 | 94908 | |
| 777 | | | | | | | | | 101172 | 103087 | |
| 778 | | | | | | | | | 24694 | 33804 | |
| 779 | | | | | | | | | 261749 | 270859 | |
| 780 | | | | | | | | | 11963 | 13878 | |
| 781 | | | | | | | | | 11673 | 14353 | |
| 782 | | | | | | | | | 11661 | 14339 | |
| 783 | | | | | | | | | 37 | 2714 | |
| 784 | | | | | | | | | 11671 | 14348 | |
| 785 | | | | | | | | | 1494 | 4171 | |
| 786 | | | | | | | | | 10184 | 12273 | |
| 787 | | | | | | | | | 51656 | 53785 | |
| 788 | | | | | | | | | 368690 | 371206 | |
| 789 | | | | | | | | | 20762 | 22677 | |
| 790 | | | | | | | | | 408 | 2323 | |
| 791 | | | | | | | | | 57709 | 59078 | |
| 792 | | | | | | | | | 852 | 2259 | |
| 793 | | | | | | | | | 402013 | 404602 | Additional ThsA homolog KEQ37438.1 |
| 794 | | | | | | | | | 534537 | 537500 | |
| 795 | | | | | | | | | 54006 | 55948 | |
| 796 | | | | | | | | | 268 | 2210 | |
| 797 | KFF62087.1 | 15092 | 20449 | 278 | | | | | 278870 | 286182 | |
| 798 | | | | | | | | | 13328 | 15398 | |
| 799 | | | | | | | | | 29271 | 31828 | |
| 800 | | | | | | | | | 1177641 | 1180423 | |
| 801 | | | | | | | | | 796009 | 798073 | |
| 802 | | | | | | | | | 25866 | 35282 | |
| 803 | | | | | | | | | 14362 | 17169 | |
| 804 | | | | | | | | | 43124 | 45703 | |
| 805 | | | | | | | | | 1357 | 3279 | |
| 806 | | | | | | | | | 14786 | 17593 | |
| 807 | | | | | | | | | 80211 | 82768 | Additional ThsA homolog KIE90699.1 and KIE90686.1 |
| 808 | | | | | | | | | 80286 | 82844 | Additional ThsA homolog KIE90603.1 |
| 809 | | | | | | | | | 80290 | 82848 | Additional ThsA homolog KIE97286.1 |
| 810 | | | | | | | | | 25818 | 28376 | Additional ThsA homolog KIE97673.1 |
| 811 | | | | | | | | | 25803 | 28361 | Additional ThsA homolog KIE96200.1 |
| 812 | | | | | | | | | 80213 | 82771 | Additional ThsA homolog KIE90358.1 |
| 813 | KHN56275.1 | 15137 | 20494 | 183 | | | | | 212601 | 218790 | |
| 814 | | | | | | | | | 89226 | 92441 | |
| 815 | | | | | | | | | 548602 | 550666 | |
| 816 | | | | | | | | | 791385 | 792765 | |
| 817 | | | | | | | | | 4381185 | 4383127 | |
| 818 | | | | | | | | | 79011 | 80966 | |
| 819 | | | | | | | | | 79815 | 81770 | |
| 820 | | | | | | | | | 57400 | 59342 | |
| 821 | | | | | | | | | 343540 | 345493 | |
| 822 | | | | | | | | | 303729 | 307800 | |
| 823 | | | | | | | | | 268769 | 271576 | |
| 824 | | | | | | | | | 540414 | 543221 | |
| 825 | | | | | | | | | 119577 | 122384 | |
| 826 | | | | | | | | | 551887 | 554694 | |
| 827 | | | | | | | | | 743854 | 746661 | |
| 828 | | | | | | | | | 119853 | 122660 | |
| 829 | | | | | | | | | 28372 | 37080 | |
| 830 | | | | | | | | | 1890562 | 1892626 | |
| 831 | | | | | | | | | 923150 | 925214 | |
| 832 | | | | | | | | | 766850 | 768914 | |
| 833 | | | | | | | | | 2675060 | 2675910 | |
| 834 | | | | | | | | | 979222 | 981286 | |
| 835 | | | | | | | | | 1470124 | 1472188 | |
| 836 | | | | | | | | | 93913 | 98742 | Additional ThsA homolog ENU62563.1 |
| 837 | | | | | | | | | 97812 | 100703 | |
| 838 | | | | | | | | | 1497967 | 1506125 | |
| 839 | | | | | | | | | 170794 | 174348 | Additional ThsA homolog ENX27879.1 |
| 840 | | | | | | | | | 676899 | 679468 | |
| 841 | | | | | | | | | 1866683 | 1870168 | |
| 842 | | | | | | | | | 214685 | 217492 | |
| 843 | | | | | | | | | 121299 | 124106 | |
| 844 | | | | | | | | | 1399298 | 1402105 | |
| 845 | | | | | | | | | 123919 | 126726 | |
| 846 | | | | | | | | | 122496 | 125303 | |
| 847 | | | | | | | | | 122329 | 125136 | |
| 848 | | | | | | | | | 448160 | 450967 | |
| 849 | | | | | | | | | 575596 | 578403 | |
| 850 | | | | | | | | | 401264 | 404071 | |
| 851 | | | | | | | | | 254055 | 256166 | |
| 852 | | | | | | | | | 800671 | 802590 | |
| 853 | | | | | | | | | 253248 | 255167 | |
| 854 | | | | | | | | | 476 | 3696 | |
| 855 | | | | | | | | | 212236 | 214349 | |
| 856 | | | | | | | | | 1791824 | 1793888 | |
| 857 | | | | | | | | | 773643 | 775707 | |
| 858 | | | | | | | | | 434828 | 436769 | |
| 859 | | | | | | | | | 397991 | 399932 | |
| 860 | | | | | | | | | 449083 | 451024 | |
| 861 | | | | | | | | | 95 | 2309 | |
| 862 | | | | | | | | | 1377455 | 1381181 | |
| 863 | | | | | | | | | 2693429 | 2697155 | |
| 864 | | | | | | | | | 737416 | 740613 | |
| 865 | | | | | | | | | 1907694 | 1910501 | |
| 866 | | | | | | | | | 1393914 | 1395856 | |
| 867 | | | | | | | | | 103489 | 105607 | |
| 868 | | | | | | | | | 53375 | 55317 | |
| 869 | | | | | | | | | 89124 | 92339 | |
| 870 | | | | | | | | | 58887 | 62102 | |
| 871 | | | | | | | | | 88416 | 91631 | |
| 872 | | | | | | | | | 58887 | 62102 | |
| 873 | | | | | | | | | 58887 | 62102 | |
| 874 | | | | | | | | | 58887 | 62102 | |
| 875 | | | | | | | | | 89258 | 92473 | |
| 876 | | | | | | | | | 59082 | 62297 | |
| 877 | | | | | | | | | 88152 | 91367 | |
| 878 | | | | | | | | | 58887 | 62102 | |
| 879 | | | | | | | | | 89302 | 92517 | |
| 880 | | | | | | | | | 59082 | 62297 | |
| 881 | | | | | | | | | 3860 | 7075 | |
| 882 | | | | | | | | | 58887 | 62102 | |
| 883 | | | | | | | | | 2496 | 5711 | |
| 884 | | | | | | | | | 58887 | 62102 | |
| 885 | | | | | | | | | 89302 | 92517 | |
| 886 | | | | | | | | | 88596 | 91811 | |
| 887 | | | | | | | | | 2217 | 5432 | |
| 888 | | | | | | | | | 89258 | 92473 | |
| 889 | | | | | | | | | 58887 | 62102 | |
| 890 | | | | | | | | | 59471 | 62686 | |
| 891 | | | | | | | | | 76731 | 79946 | |
| 892 | | | | | | | | | 36126 | 39341 | |
| 893 | | | | | | | | | 89173 | 92388 | |
| 894 | | | | | | | | | 89173 | 92388 | |
| 895 | | | | | | | | | 89124 | 92339 | |
| 896 | | | | | | | | | 58887 | 62102 | |
| 897 | | | | | | | | | 89258 | 92472 | Additional ThsA homolog EZV09968.1 |
| 898 | | | | | | | | | 58887 | 62102 | |
| 899 | | | | | | | | | 79669 | 82884 | |
| 900 | | | | | | | | | 89302 | 92517 | |
| 901 | | | | | | | | | 58475 | 61690 | |
| 902 | | | | | | | | | 58887 | 62102 | |
| 903 | | | | | | | | | 58887 | 62102 | |
| 904 | | | | | | | | | 89192 | 92407 | |
| 905 | | | | | | | | | 58887 | 62102 | |
| 906 | | | | | | | | | 89155 | 92370 | |
| 907 | | | | | | | | | 64585 | 67800 | |
| 908 | | | | | | | | | 89258 | 92473 | |
| 909 | | | | | | | | | 88340 | 91555 | |
| 910 | | | | | | | | | 89192 | 92407 | |
| 911 | | | | | | | | | 58821 | 62036 | |
| 912 | | | | | | | | | 58887 | 62102 | |
| 913 | | | | | | | | | 3860 | 7075 | |
| 914 | | | | | | | | | 58765 | 61980 | |
| 915 | | | | | | | | | 58887 | 62101 | Additional ThsA homolog EZV68413.1 |
| 916 | | | | | | | | | 88570 | 91785 | |
| 917 | | | | | | | | | 89253 | 92468 | |
| 918 | | | | | | | | | 58887 | 62102 | |
| 919 | | | | | | | | | 58887 | 62102 | |
| 920 | | | | | | | | | 59048 | 62263 | |
| 921 | | | | | | | | | 35074 | 38289 | |
| 922 | | | | | | | | | 64681 | 67896 | |
| 923 | | | | | | | | | 58887 | 62102 | |
| 924 | | | | | | | | | 88826 | 92041 | |
| 925 | | | | | | | | | 88334 | 91549 | |
| 926 | | | | | | | | | 3114 | 6329 | |
| 927 | | | | | | | | | 58887 | 62102 | |
| 928 | | | | | | | | | 58887 | 62102 | |
| 929 | | | | | | | | | 59079 | 62294 | |
| 930 | | | | | | | | | 3091 | 6306 | |
| 931 | | | | | | | | | 3091 | 6306 | |
| 932 | | | | | | | | | 58887 | 62102 | |
| 933 | | | | | | | | | 59099 | 62314 | |
| 934 | | | | | | | | | 58653 | 61868 | |
| 935 | | | | | | | | | 58887 | 62102 | |
| 936 | | | | | | | | | 89173 | 92388 | |
| 937 | | | | | | | | | 89168 | 92383 | |
| 938 | | | | | | | | | 58887 | 62102 | |
| 939 | | | | | | | | | 89258 | 92473 | |
| 940 | | | | | | | | | 89258 | 92473 | |
| 941 | | | | | | | | | 58887 | 62102 | |
| 942 | | | | | | | | | 58887 | 62102 | |
| 943 | | | | | | | | | 58887 | 62102 | |
| 944 | | | | | | | | | 58887 | 62102 | |
| 945 | | | | | | | | | 58653 | 61868 | |
| 946 | | | | | | | | | 89028 | 92243 | |
| 947 | | | | | | | | | 3800 | 7015 | |
| 948 | | | | | | | | | 87505 | 90720 | |
| 949 | | | | | | | | | 59081 | 62296 | |
| 950 | | | | | | | | | 58655 | 61870 | |
| 951 | | | | | | | | | 89301 | 92515 | |
| 952 | | | | | | | | | 58887 | 62102 | |
| 953 | | | | | | | | | 58887 | 62102 | |
| 954 | | | | | | | | | 58887 | 62102 | |
| 955 | | | | | | | | | 3114 | 6329 | |
| 956 | | | | | | | | | 58887 | 62102 | |
| 957 | | | | | | | | | 89302 | 92517 | |
| 958 | | | | | | | | | 51851 | 55066 | |
| 959 | | | | | | | | | 89258 | 92473 | |
| 960 | | | | | | | | | 58887 | 62102 | |
| 961 | | | | | | | | | 58887 | 62102 | |
| 962 | | | | | | | | | 59503 | 62718 | |
| 963 | | | | | | | | | 58887 | 62102 | |
| 964 | | | | | | | | | 40472 | 43687 | |
| 965 | | | | | | | | | 58887 | 62102 | |
| 966 | | | | | | | | | 58907 | 62122 | |
| 967 | | | | | | | | | 26695 | 29910 | |
| 968 | | | | | | | | | 58887 | 62102 | |
| 969 | | | | | | | | | 89798 | 93013 | |
| 970 | | | | | | | | | 58887 | 62102 | |
| 971 | | | | | | | | | 58887 | 62102 | |
| 972 | | | | | | | | | 89301 | 92515 | Additional ThsA homolog EZX30780.1 |
| 973 | | | | | | | | | 58653 | 61867 | Additional ThsA homolog EZX33811.1 |
| 974 | | | | | | | | | 89253 | 92467 | Additional ThsA homolog EZX38407.1 |
| 975 | | | | | | | | | 89258 | 92473 | |
| 976 | | | | | | | | | 6173 | 9387 | |
| 977 | | | | | | | | | 58887 | 62101 | |
| 978 | | | | | | | | | 85412 | 88627 | |
| 979 | | | | | | | | | 58887 | 62102 | |
| 980 | | | | | | | | | 58887 | 62102 | |
| 981 | | | | | | | | | 58887 | 62101 | Additional ThsA homolog EZX53549.1 |
| 982 | | | | | | | | | 89302 | 92517 | |
| 983 | | | | | | | | | 59081 | 62296 | |
| 984 | | | | | | | | | 58887 | 62102 | |
| 985 | | | | | | | | | 63625 | 66839 | Additional ThsA homolog EZX64344.1 |
| 986 | | | | | | | | | 58887 | 62102 | |
| 987 | | | | | | | | | 58821 | 62036 | |
| 988 | | | | | | | | | 58887 | 62102 | |
| 989 | | | | | | | | | 8858 | 12073 | |
| 990 | | | | | | | | | 89257 | 92472 | |
| 991 | | | | | | | | | 89302 | 92517 | |
| 992 | | | | | | | | | 58887 | 62102 | |
| 993 | | | | | | | | | 63857 | 67072 | |
| 994 | | | | | | | | | 87357 | 90572 | |
| 995 | | | | | | | | | 89258 | 92473 | |
| 996 | | | | | | | | | 58887 | 62102 | |
| 997 | | | | | | | | | 89214 | 92429 | |
| 998 | | | | | | | | | 89253 | 91325 | |
| 999 | | | | | | | | | 89168 | 92383 | |
| 1000 | | | | | | | | | 58887 | 62102 | |
| 1001 | | | | | | | | | 48320 | 51535 | |
| 1002 | | | | | | | | | 89302 | 92517 | |
| 1003 | | | | | | | | | 29770 | 32985 | |
| 1004 | | | | | | | | | 89304 | 92521 | |
| 1005 | | | | | | | | | 58887 | 62102 | |
| 1006 | | | | | | | | | 89253 | 92468 | |
| 1007 | | | | | | | | | 58887 | 62102 | |
| 1008 | | | | | | | | | 89253 | 91412 | |
| 1009 | | | | | | | | | 2625 | 5840 | |
| 1010 | | | | | | | | | 88832 | 92047 | |
| 1011 | | | | | | | | | 89302 | 92517 | |
| 1012 | | | | | | | | | 89302 | 92517 | |
| 1013 | | | | | | | | | 58887 | 62102 | |
| 1014 | | | | | | | | | 89216 | 92431 | |
| 1015 | | | | | | | | | 58887 | 62102 | |
| 1016 | | | | | | | | | 64320 | 67534 | Additional ThsA homolog EZY44866.1 |
| 1017 | | | | | | | | | 58887 | 62102 | |
| 1018 | | | | | | | | | 58887 | 62102 | |
| 1019 | | | | | | | | | 58887 | 62101 | Additional ThsA homolog EZY52188.1 |
| 1020 | | | | | | | | | 58887 | 62101 | Additional ThsA homolog EZY54363.1 |
| 1021 | | | | | | | | | 4154 | 7368 | Additional ThsA homolog EZY58963.1 |
| 1022 | | | | | | | | | 58887 | 62102 | |
| 1023 | | | | | | | | | 58887 | 62102 | |
| 1024 | | | | | | | | | 89258 | 92473 | |
| 1025 | | | | | | | | | 58887 | 62102 | |
| 1026 | | | | | | | | | 58887 | 62102 | |
| 1027 | | | | | | | | | 89302 | 92517 | |
| 1028 | | | | | | | | | 58887 | 62102 | |
| 1029 | | | | | | | | | 58821 | 62036 | |
| 1030 | | | | | | | | | 58831 | 62046 | |
| 1031 | | | | | | | | | 89253 | 92468 | |
| 1032 | | | | | | | | | 14360 | 17575 | |
| 1033 | | | | | | | | | 86376 | 89591 | |
| 1034 | | | | | | | | | 89253 | 92468 | |
| 1035 | | | | | | | | | 58887 | 61350 | |
| 1036 | | | | | | | | | 58821 | 62036 | |
| 1037 | | | | | | | | | 58887 | 61350 | |
| 1038 | | | | | | | | | 3244 | 6459 | |
| 1039 | | | | | | | | | 58887 | 62101 | Additional ThsA homolog EZZ07027.1 |
| 1040 | | | | | | | | | 3860 | 7075 | |
| 1041 | | | | | | | | | 89258 | 92474 | |
| 1042 | | | | | | | | | 58887 | 62102 | |
| 1043 | | | | | | | | | 58887 | 62101 | |
| 1044 | | | | | | | | | 89028 | 92243 | |
| 1045 | | | | | | | | | 57669 | 60884 | |
| 1046 | | | | | | | | | 58887 | 62102 | |
| 1047 | | | | | | | | | 57242 | 60457 | |
| 1048 | | | | | | | | | 11497 | 14712 | |
| 1049 | | | | | | | | | 58887 | 62102 | |
| 1050 | | | | | | | | | 89603 | 92818 | |
| 1051 | | | | | | | | | 14572 | 17787 | |
| 1052 | | | | | | | | | 58887 | 62102 | |
| 1053 | | | | | | | | | 58887 | 62102 | |
| 1054 | | | | | | | | | 88413 | 91628 | |
| 1055 | | | | | | | | | 59194 | 62409 | |
| 1056 | | | | | | | | | 58887 | 62102 | |
| 1057 | | | | | | | | | 89186 | 92401 | |
| 1058 | | | | | | | | | 58887 | 62102 | |
| 1059 | | | | | | | | | 89001 | 92216 | |
| 1060 | | | | | | | | | 58887 | 62101 | Additional ThsA homolog EZZ66975.1 |
| 1061 | | | | | | | | | 58887 | 62102 | |
| 1062 | | | | | | | | | 89304 | 92519 | |
| 1063 | | | | | | | | | 58887 | 62102 | |
| 1064 | | | | | | | | | 89160 | 92375 | |
| 1065 | | | | | | | | | 58830 | 62045 | |
| 1066 | | | | | | | | | 89217 | 92432 | |
| 1067 | | | | | | | | | 58887 | 62102 | |
| 1068 | | | | | | | | | 58821 | 62036 | |
| 1069 | | | | | | | | | 58821 | 62036 | |
| 1070 | | | | | | | | | 58887 | 62102 | |
| 1071 | | | | | | | | | 86561 | 89776 | |
| 1072 | | | | | | | | | 58887 | 62102 | |
| 1073 | | | | | | | | | 89302 | 92517 | |
| 1074 | | | | | | | | | 58887 | 62102 | |
| 1075 | | | | | | | | | 86128 | 89343 | |
| 1076 | | | | | | | | | 58886 | 62101 | |
| 1077 | | | | | | | | | 72244 | 75459 | |
| 1078 | | | | | | | | | 58887 | 62102 | |
| 1079 | | | | | | | | | 89253 | 92468 | |
| 1080 | | | | | | | | | 58828 | 62043 | |
| 1081 | | | | | | | | | 58887 | 62102 | |
| 1082 | | | | | | | | | 58887 | 62102 | |
| 1083 | | | | | | | | | 58887 | 62102 | |
| 1084 | | | | | | | | | 58887 | 62102 | |
| 1085 | | | | | | | | | 89217 | 92432 | |
| 1086 | | | | | | | | | 89168 | 92383 | |
| 1087 | | | | | | | | | 89216 | 92431 | |
| 1088 | | | | | | | | | 58821 | 62036 | |
| 1089 | | | | | | | | | 58887 | 62102 | |
| 1090 | | | | | | | | | 58887 | 62102 | |
| 1091 | | | | | | | | | 58821 | 62036 | |
| 1092 | | | | | | | | | 58887 | 62101 | |
| 1093 | | | | | | | | | 1088 | 4303 | |
| 1094 | | | | | | | | | 89302 | 92517 | |
| 1095 | | | | | | | | | 89258 | 92473 | |
| 1096 | | | | | | | | | 58887 | 62102 | |
| 1097 | | | | | | | | | 58886 | 62101 | |
| 1098 | | | | | | | | | 58886 | 62101 | |
| 1099 | | | | | | | | | 89236 | 92451 | |
| 1100 | | | | | | | | | 58887 | 62102 | |
| 1101 | | | | | | | | | 58887 | 62102 | |
| 1102 | | | | | | | | | 58887 | 62102 | |
| 1103 | | | | | | | | | 58887 | 62102 | |
| 1104 | | | | | | | | | 11491 | 14706 | |
| 1105 | | | | | | | | | 73114 | 76329 | |
| 1106 | | | | | | | | | 58821 | 62036 | |
| 1107 | | | | | | | | | 58887 | 62102 | |
| 1108 | | | | | | | | | 27936 | 31151 | |
| 1109 | | | | | | | | | 58887 | 62102 | |
| 1110 | | | | | | | | | 58887 | 62102 | |
| 1111 | | | | | | | | | 89217 | 92432 | |
| 1112 | | | | | | | | | 86600 | 89815 | |
| 1113 | | | | | | | | | 58887 | 62102 | |
| 1114 | | | | | | | | | 58821 | 62036 | |
| 1115 | | | | | | | | | 58887 | 62102 | |
| 1116 | | | | | | | | | 58821 | 62036 | |
| 1117 | | | | | | | | | 58821 | 62036 | |
| 1118 | | | | | | | | | 58887 | 62102 | |
| 1119 | | | | | | | | | 58887 | 62102 | |
| 1120 | | | | | | | | | 90149 | 93364 | |
| 1121 | | | | | | | | | 89243 | 92458 | |
| 1122 | | | | | | | | | 58887 | 62102 | |
| 1123 | | | | | | | | | 58887 | 62102 | |
| 1124 | | | | | | | | | 58886 | 62101 | |
| 1125 | | | | | | | | | 89302 | 92517 | |
| 1126 | | | | | | | | | 86097 | 89312 | |
| 1127 | | | | | | | | | 89258 | 92473 | |
| 1128 | | | | | | | | | 58887 | 62102 | |
| 1129 | | | | | | | | | 89253 | 92468 | |
| 1130 | | | | | | | | | 58887 | 62102 | |
| 1131 | | | | | | | | | 23191 | 26406 | |
| 1132 | | | | | | | | | 58887 | 62102 | |
| 1133 | | | | | | | | | 58887 | 62102 | |
| 1134 | | | | | | | | | 58887 | 61046 | |
| 1135 | | | | | | | | | 58887 | 62102 | |
| 1136 | | | | | | | | | 28112 | 30271 | |
| 1137 | | | | | | | | | 58886 | 62101 | |
| 1138 | | | | | | | | | 58821 | 62036 | |
| 1139 | | | | | | | | | 58886 | 62101 | |
| 1140 | | | | | | | | | 58887 | 62102 | |
| 1141 | | | | | | | | | 89302 | 92517 | |
| 1142 | | | | | | | | | 58887 | 62102 | |
| 1143 | | | | | | | | | 58887 | 62102 | |
| 1144 | | | | | | | | | 89258 | 92473 | |
| 1145 | | | | | | | | | 89302 | 92517 | |
| 1146 | | | | | | | | | 89258 | 92473 | |
| 1147 | | | | | | | | | 26639 | 29854 | |
| 1148 | | | | | | | | | 58821 | 62036 | |
| 1149 | | | | | | | | | 14243 | 17458 | |
| 1150 | | | | | | | | | 58887 | 62102 | |
| 1151 | | | | | | | | | 58887 | 62102 | |
| 1152 | | | | | | | | | 89217 | 92432 | |
| 1153 | | | | | | | | | 58886 | 62101 | |
| 1154 | | | | | | | | | 89303 | 92518 | |
| 1155 | | | | | | | | | 58886 | 62101 | |
| 1156 | | | | | | | | | 58887 | 61046 | |
| 1157 | | | | | | | | | 7935 | 11150 | |
| 1158 | | | | | | | | | 7491 | 10706 | |
| 1159 | | | | | | | | | 58887 | 62102 | |
| 1160 | | | | | | | | | 89302 | 92517 | |
| 1161 | | | | | | | | | 89283 | 92498 | |
| 1162 | | | | | | | | | 3990 | 6149 | |
| 1163 | | | | | | | | | 58887 | 62102 | |
| 1164 | | | | | | | | | 3990 | 6149 | |
| 1165 | | | | | | | | | 89302 | 92517 | |
| 1166 | | | | | | | | | 89173 | 92388 | |
| 1167 | | | | | | | | | 89167 | 92382 | |
| 1168 | | | | | | | | | 58887 | 62102 | |
| 1169 | | | | | | | | | 29744 | 32959 | |
| 1170 | | | | | | | | | 58821 | 62036 | |
| 1171 | | | | | | | | | 86117 | 89332 | |
| 1172 | | | | | | | | | 89302 | 92517 | |
| 1173 | | | | | | | | | 58887 | 62102 | |
| 1174 | | | | | | | | | 58887 | 62102 | |
| 1175 | | | | | | | | | 59150 | 62365 | |
| 1176 | | | | | | | | | 89257 | 92472 | |
| 1177 | | | | | | | | | 58887 | 61046 | |
| 1178 | | | | | | | | | 89303 | 91462 | |
| 1179 | | | | | | | | | 58887 | 62102 | |
| 1180 | | | | | | | | | 58887 | 62102 | |
| 1181 | | | | | | | | | 58831 | 62046 | |
| 1182 | | | | | | | | | 58887 | 62102 | |
| 1183 | | | | | | | | | 58887 | 62102 | |
| 1184 | | | | | | | | | 59012 | 62227 | |
| 1185 | | | | | | | | | 58887 | 62102 | |
| 1186 | | | | | | | | | 42602 | 45817 | |
| 1187 | | | | | | | | | 58821 | 62036 | |
| 1188 | | | | | | | | | 58821 | 62036 | |
| 1189 | | | | | | | | | 86600 | 89815 | |
| 1190 | | | | | | | | | 58887 | 62102 | |
| 1191 | | | | | | | | | 14572 | 17787 | |
| 1192 | | | | | | | | | 58887 | 62102 | |
| 1193 | | | | | | | | | 88664 | 91879 | |
| 1194 | | | | | | | | | 58887 | 62102 | |
| 1195 | | | | | | | | | 89172 | 92386 | |
| 1196 | | | | | | | | | 87289 | 90503 | |
| 1197 | | | | | | | | | 89172 | 92386 | |
| 1198 | | | | | | | | | 58821 | 62036 | |
| 1199 | | | | | | | | | 89216 | 92430 | |
| 1200 | | | | | | | | | 58887 | 62101 | |
| 1201 | | | | | | | | | 2720 | 5935 | |
| 1202 | | | | | | | | | 58887 | 62101 | |
| 1203 | | | | | | | | | 58887 | 62101 | |
| 1204 | | | | | | | | | 58888 | 62103 | |
| 1205 | | | | | | | | | 58887 | 62102 | |
| 1206 | | | | | | | | | 89173 | 92388 | |
| 1207 | | | | | | | | | 86562 | 89777 | |
| 1208 | | | | | | | | | 89257 | 92472 | |
| 1209 | | | | | | | | | 2720 | 5935 | |
| 1210 | | | | | | | | | 58886 | 62101 | |
| 1211 | | | | | | | | | 87715 | 90930 | |
| 1212 | | | | | | | | | 17386 | 20601 | |
| 1213 | | | | | | | | | 58887 | 62102 | |
| 1214 | | | | | | | | | 89253 | 92468 | |
| 1215 | | | | | | | | | 89302 | 92517 | |
| 1216 | | | | | | | | | 58764 | 61979 | |
| 1217 | | | | | | | | | 58652 | 61867 | |
| 1218 | | | | | | | | | 58886 | 62101 | |
| 1219 | | | | | | | | | 58887 | 62102 | |
| 1220 | | | | | | | | | 58887 | 62102 | |
| 1221 | | | | | | | | | 58887 | 62102 | |
| 1222 | | | | | | | | | 58887 | 62102 | |
| 1223 | | | | | | | | | 58887 | 62102 | |
| 1224 | | | | | | | | | 11769 | 14984 | |
| 1225 | | | | | | | | | 58877 | 62092 | |
| 1226 | | | | | | | | | 329 | 2488 | |
| 1227 | | | | | | | | | 329 | 2488 | |
| 1228 | | | | | | | | | 89258 | 92473 | |
| 1229 | | | | | | | | | 73104 | 76319 | |
| 1230 | | | | | | | | | 11491 | 14706 | |
| 1231 | | | | | | | | | 58887 | 62102 | |
| 1232 | | | | | | | | | 58887 | 62102 | |
| 1233 | | | | | | | | | 29744 | 32959 | |
| 1234 | | | | | | | | | 58887 | 62102 | |
| 1235 | | | | | | | | | 58887 | 62102 | |
| 1236 | | | | | | | | | 58887 | 62102 | |
| 1237 | | | | | | | | | 58821 | 62036 | |
| 1238 | | | | | | | | | 58829 | 62044 | |
| 1239 | | | | | | | | | 58821 | 62036 | |
| 1240 | | | | | | | | | 58887 | 62102 | |
| 1241 | | | | | | | | | 58887 | 62102 | |
| 1242 | | | | | | | | | 58887 | 62102 | |
| 1243 | | | | | | | | | 58887 | 61046 | |
| 1244 | | | | | | | | | 89302 | 91461 | |
| 1245 | | | | | | | | | 58887 | 62102 | |
| 1246 | | | | | | | | | 58887 | 62102 | |
| 1247 | | | | | | | | | 89302 | 92517 | |
| 1248 | | | | | | | | | 58887 | 62102 | |
| 1249 | | | | | | | | | 65904 | 69119 | |
| 1250 | | | | | | | | | 58887 | 62102 | |
| 1251 | | | | | | | | | 58887 | 62102 | |
| 1252 | | | | | | | | | 58887 | 62102 | |
| 1253 | | | | | | | | | 14572 | 17787 | |
| 1254 | | | | | | | | | 89217 | 92432 | |
| 1255 | | | | | | | | | 58887 | 62102 | |
| 1256 | | | | | | | | | 58764 | 61979 | |
| 1257 | | | | | | | | | 86241 | 89456 | |
| 1258 | | | | | | | | | 58887 | 62102 | |
| 1259 | | | | | | | | | 58887 | 62102 | |
| 1260 | | | | | | | | | 58887 | 62102 | |
| 1261 | | | | | | | | | 58887 | 62102 | |
| 1262 | | | | | | | | | 58821 | 62036 | |
| 1263 | | | | | | | | | 89303 | 92518 | |
| 1264 | | | | | | | | | 58821 | 62036 | |
| 1265 | | | | | | | | | 58887 | 62102 | |
| 1266 | | | | | | | | | 58887 | 62102 | |
| 1267 | | | | | | | | | 58887 | 62102 | |
| 1268 | | | | | | | | | 58887 | 62102 | |
| 1269 | | | | | | | | | 58887 | 62101 | Additional ThsA homolog KAF14361.1 |
| 1270 | | | | | | | | | 58886 | 62101 | |
| 1271 | | | | | | | | | 58887 | 62102 | |
| 1272 | | | | | | | | | 89302 | 92517 | |
| 1273 | | | | | | | | | 29744 | 32959 | |
| 1274 | | | | | | | | | 89302 | 92517 | |
| 1275 | | | | | | | | | 58821 | 62036 | |
| 1276 | | | | | | | | | 58887 | 62102 | |
| 1277 | | | | | | | | | 58831 | 62046 | |
| 1278 | | | | | | | | | 58887 | 62102 | |
| 1279 | | | | | | | | | 73077 | 76292 | |
| 1280 | | | | | | | | | 73866 | 77081 | |
| 1281 | | | | | | | | | 58530 | 61745 | |
| 1282 | | | | | | | | | 58887 | 62102 | |
| 1283 | | | | | | | | | 329 | 2488 | |
| 1284 | | | | | | | | | 58821 | 62036 | |
| 1285 | | | | | | | | | 89173 | 92388 | |
| 1286 | | | | | | | | | 58887 | 62102 | |
| 1287 | | | | | | | | | 58887 | 62102 | |
| 1288 | | | | | | | | | 58887 | 62102 | |
| 1289 | | | | | | | | | 58887 | 62102 | |
| 1290 | | | | | | | | | 58887 | 62102 | |
| 1291 | | | | | | | | | 58829 | 62044 | |
| 1292 | | | | | | | | | 58829 | 62044 | |
| 1293 | | | | | | | | | 58887 | 62102 | |
| 1294 | | | | | | | | | 58831 | 62046 | |
| 1295 | | | | | | | | | 2720 | 5935 | |
| 1296 | | | | | | | | | 58887 | 62102 | |
| 1297 | | | | | | | | | 89168 | 92383 | |
| 1298 | | | | | | | | | 58887 | 62102 | |
| 1299 | | | | | | | | | 89253 | 92468 | |
| 1300 | | | | | | | | | 89258 | 91417 | |
| 1301 | | | | | | | | | 89302 | 91461 | |
| 1302 | | | | | | | | | 52248 | 55463 | |
| 1303 | | | | | | | | | 58821 | 62036 | |
| 1304 | | | | | | | | | 58887 | 62102 | |
| 1305 | | | | | | | | | 7905 | 11120 | |
| 1306 | | | | | | | | | 35425 | 38640 | |
| 1307 | | | | | | | | | 89142 | 92357 | |
| 1308 | | | | | | | | | 35344 | 38559 | |
| 1309 | | | | | | | | | 58887 | 62102 | |
| 1310 | | | | | | | | | 7905 | 11120 | |
| 1311 | | | | | | | | | 7905 | 11120 | |
| 1312 | | | | | | | | | 58887 | 62102 | |
| 1313 | | | | | | | | | 58887 | 62102 | |
| 1314 | | | | | | | | | 17266 | 20481 | |
| 1315 | | | | | | | | | 89142 | 92357 | |
| 1316 | | | | | | | | | 89180 | 92395 | |
| 1317 | | | | | | | | | 89217 | 92432 | |
| 1318 | | | | | | | | | 58887 | 62102 | |
| 1319 | | | | | | | | | 58887 | 62102 | |
| 1320 | | | | | | | | | 89216 | 92431 | |
| 1321 | | | | | | | | | 87144 | 90359 | |
| 1322 | | | | | | | | | 58887 | 62102 | |
| 1323 | | | | | | | | | 11773 | 14988 | |
| 1324 | | | | | | | | | 89258 | 92473 | |
| 1325 | | | | | | | | | 59129 | 62344 | |
| 1326 | | | | | | | | | 58887 | 62102 | |
| 1327 | | | | | | | | | 89301 | 92516 | |
| 1328 | | | | | | | | | 58887 | 62102 | |
| 1329 | | | | | | | | | 58887 | 62102 | |
| 1330 | | | | | | | | | 58887 | 62102 | |
| 1331 | | | | | | | | | 58887 | 62102 | |
| 1332 | | | | | | | | | 89302 | 92517 | |
| 1333 | | | | | | | | | 58831 | 62046 | |
| 1334 | | | | | | | | | 3137 | 6352 | |
| 1335 | | | | | | | | | 58887 | 62102 | |
| 1336 | | | | | | | | | 58887 | 62102 | |
| 1337 | | | | | | | | | 58887 | 62102 | |
| 1338 | | | | | | | | | 50208 | 53423 | |
| 1339 | | | | | | | | | 88375 | 91590 | |
| 1340 | | | | | | | | | 58887 | 62102 | |
| 1341 | | | | | | | | | 58887 | 62102 | |
| 1342 | | | | | | | | | 58887 | 62101 | |
| 1343 | | | | | | | | | 58888 | 62103 | |
| 1344 | | | | | | | | | 58888 | 62103 | |
| 1345 | | | | | | | | | 58821 | 62036 | |
| 1346 | | | | | | | | | 58887 | 62102 | |
| 1347 | | | | | | | | | 58764 | 61979 | |
| 1348 | | | | | | | | | 58887 | 62102 | |
| 1349 | | | | | | | | | 58887 | 62102 | |
| 1350 | | | | | | | | | 58887 | 62102 | |
| 1351 | | | | | | | | | 89258 | 92473 | |
| 1352 | | | | | | | | | 58887 | 62102 | |
| 1353 | | | | | | | | | 89302 | 92517 | |
| 1354 | | | | | | | | | 58887 | 62102 | |
| 1355 | | | | | | | | | 58890 | 62105 | |
| 1356 | | | | | | | | | 58887 | 62102 | |
| 1357 | | | | | | | | | 58887 | 62102 | |
| 1358 | | | | | | | | | 58887 | 61046 | |
| 1359 | | | | | | | | | 58887 | 62102 | |
| 1360 | | | | | | | | | 15133 | 18348 | |
| 1361 | | | | | | | | | 58887 | 62102 | |
| 1362 | | | | | | | | | 58887 | 62102 | |
| 1363 | | | | | | | | | 58887 | 62102 | |
| 1364 | | | | | | | | | 58887 | 62102 | |
| 1365 | | | | | | | | | 58887 | 62102 | |
| 1366 | | | | | | | | | 58887 | 62102 | |
| 1367 | | | | | | | | | 89215 | 92430 | |
| 1368 | | | | | | | | | 89173 | 92388 | |
| 1369 | | | | | | | | | 58887 | 62102 | |
| 1370 | | | | | | | | | 58887 | 62102 | |
| 1371 | | | | | | | | | 11492 | 14706 | Additional ThsA homolog KAI16288.1 |
| 1372 | | | | | | | | | 2720 | 5935 | |
| 1373 | | | | | | | | | 58887 | 62102 | |
| 1374 | | | | | | | | | 58887 | 62101 | Additional ThsA homolog KAI26858.1 |
| 1375 | | | | | | | | | 58887 | 62101 | Additional ThsA homolog KAI29885.1 |
| 1376 | | | | | | | | | 58887 | 62102 | |
| 1377 | | | | | | | | | 58887 | 62102 | |
| 1378 | | | | | | | | | 58887 | 62102 | |
| 1379 | | | | | | | | | 89266 | 92481 | |
| 1380 | | | | | | | | | 58887 | 62102 | |
| 1381 | | | | | | | | | 89181 | 92396 | |
| 1382 | | | | | | | | | 89181 | 92396 | |
| 1383 | | | | | | | | | 58887 | 62102 | |
| 1384 | | | | | | | | | 5826 | 9041 | |
| 1385 | | | | | | | | | 58821 | 62036 | |
| 1386 | | | | | | | | | 59195 | 62410 | |
| 1387 | | | | | | | | | 17302 | 20517 | |
| 1388 | | | | | | | | | 58887 | 62101 | Additional ThsA homolog KAI62094.1 |
| 1389 | | | | | | | | | 58887 | 62102 | |
| 1390 | | | | | | | | | 89257 | 92472 | |
| 1391 | | | | | | | | | 23094 | 26309 | |
| 1392 | | | | | | | | | 89302 | 92517 | |
| 1393 | | | | | | | | | 58887 | 62102 | |
| 1394 | | | | | | | | | 58887 | 62102 | |
| 1395 | | | | | | | | | 58887 | 62102 | |
| 1396 | | | | | | | | | 58887 | 62102 | |
| 1397 | | | | | | | | | 2720 | 5935 | |
| 1398 | | | | | | | | | 605684 | 608266 | |
| 1399 | | | | | | | | | 2378987 | 2381503 | |
| 1400 | | | | | | | | | 310640 | 313504 | |
| 1401 | | | | | | | | | 1255726 | 1257790 | |
| 1402 | | | | | | | | | 3826893 | 3828835 | |
| 1403 | | | | | | | | | 1485 | 3579 | |
| 1404 | | | | | | | | | 639688 | 643130 | |
| 1405 | | | | | | | | | 94340 | 96396 | |
| 1406 | | | | | | | | | 1102 | 3496 | |
| 1407 | | | | | | | | | 13655 | 15725 | |
| 1408 | | | | | | | | | 20332 | 22454 | |
| 1409 | | | | | | | | | 974 | 3096 | |
| 1410 | | | | | | | | | 374357 | 376479 | |
| 1411 | | | | | | | | | 74618 | 76770 | |
| 1412 | | | | | | | | | 1153 | 3114 | |
| 1413 | | | | | | | | | 41277 | 43918 | |
| 1414 | | | | | | | | | 69089 | 71153 | |
| 1415 | | | | | | | | | 179848 | 182525 | |
| 1416 | | | | | | | | | 11882 | 13968 | |
| 1417 | | | | | | | | | 12651 | 15458 | |
| 1418 | | | | | | | | | 3676 | 7033 | |
| 1419 | | | | | | | | | 8280 | 10222 | |
| 1420 | | | | | | | | | 2504 | 6185 | |
| 1421 | | | | | | | | | 7387 | 9539 | |
| 1422 | | | | | | | | | 47043 | 48958 | |
| 1423 | | | | | | | | | 241192 | 243134 | |
| 1424 | | | | | | | | | 1908 | 3989 | |
| 1425 | | | | | | | | | 37118 | 42808 | |
| 1426 | | | | | | | | | 1106 | 3500 | |
| 1427 | | | | | | | | | 163 | 2785 | |
| 1428 | | | | | | | | | 15686 | 18266 | |
| 1429 | | | | | | | | | 46608 | 48550 | |
| 1430 | | | | | | | | | 1044 | 3541 | |
| 1431 | | | | | | | | | 8558 | 14045 | |
| 1432 | | | | | | | | | 364692 | 367369 | |
| 1433 | | | | | | | | | 28016 | 30521 | |
| 1434 | | | | | | | | | 69058 | 71661 | |
| 1435 | | | | | | | | | 123469 | 126673 | |
| 1436 | | | | | | | | | 2503 | 6286 | |
| 1437 | | | | | | | | | 10631 | 14414 | |
| 1438 | | | | | | | | | 507977 | 509953 | |
| 1439 | | | | | | | | | 259469 | 261537 | |
| 1440 | | | | | | | | | 93299 | 100962 | Additional ThsA homolog KQL33554.1 |
| 1441 | | | | | | | | | 244173 | 247473 | |
| 1442 | | | | | | | | | 145624 | 150941 | |
| 1443 | | | | | | | | | 184894 | 190211 | |
| 1444 | | | | | | | | | 4452 | 6364 | |
| 1445 | | | | | | | | | 128226 | 130138 | |
| 1446 | | | | | | | | | 298909 | 301699 | |
| 1447 | | | | | | | | | 4866 | 10841 | |
| 1448 | | | | | | | | | 204433 | 206473 | |
| 1449 | | | | | | | | | 16453 | 18427 | |
| 1450 | KSG00431.1 | 17001 | 22358 | 284 | | | | | 2213 | 6247 | |
| 1451 | KSO26598.1 | 17006 | 22363 | 284 | | | | | 124680 | 128714 | |
| 1452 | KSQ24408.1 | 17011 | 22368 | 284 | | | | | 62982 | 67016 | |
| 1453 | | | | | | | | | 49733 | 52315 | |
| 1454 | | | | | | | | | 282217 | 284200 | |
| 1455 | | | | | | | | | 208592 | 217300 | |
| 1456 | | | | | | | | | 41760 | 43858 | |
| 1457 | | | | | | | | | 156668 | 159376 | |
| 1458 | | | | | | | | | 733755 | 736830 | |
| 1459 | | | | | | | | | 77736 | 79508 | Additional ThsA homolog CEA11435.1 |
| 1460 | | | | | | | | | 77793 | 79565 | Additional ThsA homolog CEA10431.1 |
| 1461 | | | | | | | | | 1337272 | 1339830 | Additional ThsA homolog CUU52509.1 |
| 1462 | | | | | | | | | 87151 | 89092 | |
| 1463 | | | | | | | | | 746 | 3553 | |
| 1464 | | | | | | | | | 1614 | 4421 | |
| 1465 | | | | | | | | | 2106 | 4913 | |
| 1466 | | | | | | | | | 131 | 1607 | |
| 1467 | | | | | | | | | 1 | 1432 | |
| 1468 | | | | | | | | | 8 | 1931 | |
| 1469 | | | | | | | | | 1077 | 3884 | |
| 1470 | | | | | | | | | 295 | 2073 | |
| 1471 | | | | | | | | | 2520 | 5327 | |
| 1472 | | | | | | | | | 14947 | 17754 | |
| 1473 | | | | | | | | | 131 | 2938 | |
| 1474 | | | | | | | | | 4405 | 7212 | |
| 1475 | | | | | | | | | 30227 | 33460 | |
| 1476 | | | | | | | | | 244740 | 246667 | |
| 1477 | | | | | | | | | 1617 | 5976 | |
| 1478 | | | | | | | | | 445956 | 448097 | |
| 1479 | | | | | | | | | 102 | 952 | |
| 1480 | | | | | | | | | 450 | 1300 | |
| 1481 | | | | | | | | | 55284 | 63357 | |
| 1482 | | | | | | | | | 42235 | 50308 | |
| 1483 | | | | | | | | | 55284 | 63357 | |
| 1484 | | | | | | | | | 981 | 2861 | |
| 1485 | | | | | | | | | 2598 | 11358 | |
| 1486 | | | | | | | | | 7069 | 9713 | |
| 1487 | | | | | | | | | 132958 | 135057 | |
| 1488 | | | | | | | | | 8547 | 11191 | |
| 1489 | | | | | | | | | 55235 | 57150 | |
| 1490 | | | | | | | | | 935 | 3606 | |
| 1491 | | | | | | | | | 935 | 3606 | |
| 1492 | | | | | | | | | 2598 | 11358 | |
| 1493 | | | | | | | | | 81585 | 84446 | |
| 1494 | | | | | | | | | 155366 | 164406 | |
| 1495 | | | | | | | | | 80319 | 82234 | |
| 1496 | | | | | | | | | 7669 | 9584 | |
| 1497 | | | | | | | | | 7624 | 9539 | |
| 1498 | | | | | | | | | 10977 | 13784 | |
| 1499 | | | | | | | | | 32084 | 34025 | |
| 1500 | | | | | | | | | 16664 | 18605 | |
| 1501 | | | | | | | | | 9359 | 11274 | |
| 1502 | | | | | | | | | 5128 | 7069 | |
| 1503 | | | | | | | | | 5113 | 7054 | |
| 1504 | | | | | | | | | 38647 | 40562 | |
| 1505 | | | | | | | | | 9513 | 11428 | |
| 1506 | | | | | | | | | 15944 | 17885 | |
| 1507 | | | | | | | | | 124218 | 126133 | |
| 1508 | | | | | | | | | 17640 | 19555 | |
| 1509 | | | | | | | | | 2202 | 4082 | |
| 1510 | | | | | | | | | 229143 | 231058 | |
| 1511 | | | | | | | | | 2404458 | 2405838 | |
| 1512 | | | | | | | | | 817577 | 818957 | |
| 1513 | | | | | | | | | 615084 | 616464 | |
| 1514 | | | | | | | | | 152013 | 153393 | |
| 1515 | | | | | | | | | 195157 | 198738 | |
| 1516 | | | | | | | | | 10904 | 18356 | |
| 1517 | | | | | | | | | 3334527 | 3335974 | |
| 1518 | | | | | | | | | 1341773 | 1343206 | |
| 1519 | | | | | | | | | 1844605 | 1846063 | |
| 1520 | | | | | | | | | 584164 | 585641 | |
| 1521 | | | | | | | | | 134834 | 136244 | |
| 1522 | | | | | | | | | 2071675 | 2073130 | |
| 1523 | | | | | | | | | 3926192 | 3927625 | |
| 1524 | | | | | | | | | 3874800 | 3876233 | |
| 1525 | | | | | | | | | 2458217 | 2461483 | |
| 1526 | | | | | | | | | 4332366 | 4333799 | |
| 1527 | | | | | | | | | 232808 | 234255 | |
| 1528 | | | | | | | | | 160530 | 161977 | |
| 1529 | | | | | | | | | 2481111 | 2482500 | |
| 1530 | | | | | | | | | 273049 | 274496 | |
| 1531 | | | | | | | | | 2639516 | 2640982 | |
| 1532 | | | | | | | | | 2812522 | 2813955 | |
| 1533 | | | | | | | | | 97573 | 99025 | |
| 1534 | | | | | | | | | 2075549 | 2077004 | |
| 1535 | | | | | | | | | 26 | 1071 | |
| 1536 | | | | | | | | | 2133 | 3628 | |
| 1537 | | | | | | | | | 84 | 1579 | |
| 1538 | | | | | | | | | 1084123 | 1085545 | |
| 1539 | | | | | | | | | 1084123 | 1085545 | |
| 1540 | | | | | | | | | 11228 | 12661 | |
| 1541 | | | | | | | | | 33036 | 34488 | |
| 1542 | | | | | | | | | 29409 | 30859 | |
| 1543 | | | | | | | | | 224969 | 226401 | |
| 1544 | | | | | | | | | 235 | 1667 | |
| 1545 | | | | | | | | | 2635439 | 2636872 | |
| 1546 | | | | | | | | | 428631 | 430077 | |
| 1547 | | | | | | | | | 25772 | 27249 | |
| 1548 | | | | | | | | | 914 | 2336 | |
| 1549 | | | | | | | | | 2463639 | 2465086 | |
| 1550 | | | | | | | | | 1 | 862 | |
| 1551 | | | | | | | | | 26558 | 27991 | |
| 1552 | | | | | | | | | 34236 | 35669 | |
| 1553 | | | | | | | | | 3765 | 5175 | |
| 1554 | | | | | | | | | 1734329 | 1735762 | |
| 1555 | | | | | | | | | 3509 | 4956 | |
| 1556 | | | | | | | | | 2790710 | 2792159 | |
| 1557 | | | | | | | | | 157503 | 158952 | |
| 1558 | | | | | | | | | 1926980 | 1928429 | |
| 1559 | | | | | | | | | 2177471 | 2178599 | |
| 1560 | | | | | | | | | 1830 | 3263 | |
| 1561 | | | | | | | | | 11498 | 12911 | |
| 1562 | | | | | | | | | 1519 | 2929 | |
| 1563 | | | | | | | | | 76033 | 77422 | |
| 1564 | | | | | | | | | 112479 | 113937 | |
| 1565 | | | | | | | | | 11530 | 12977 | |
| 1566 | | | | | | | | | 2365 | 3812 | |
| 1567 | | | | | | | | | 2349 | 3796 | |
| 1568 | | | | | | | | | 10610 | 12057 | |
| 1569 | | | | | | | | | 1347 | 2737 | |
| 1570 | | | | | | | | | 61132 | 62524 | |
| 1571 | | | | | | | | | 88884 | 90276 | |
| 1572 | | | | | | | | | 89055 | 90447 | |
| 1573 | | | | | | | | | 25772 | 27164 | |
| 1574 | | | | | | | | | 51881 | 53291 | |
| 1575 | | | | | | | | | 190051 | 191475 | |
| 1576 | | | | | | | | | 977432 | 978898 | |
| 1577 | | | | | | | | | 33964 | 35397 | |
| 1578 | | | | | | | | | 207130 | 208563 | |
| 1579 | | | | | | | | | 308501 | 309935 | |
| 1580 | | | | | | | | | 124638 | 126071 | |
| 1581 | | | | | | | | | 75914 | 77369 | |
| 1582 | | | | | | | | | 88200 | 89655 | |
| 1583 | | | | | | | | | 76300 | 77755 | |
| 1584 | | | | | | | | | 25517 | 26932 | |
| 1585 | | | | | | | | | 1192816 | 1194306 | |
| 1586 | | | | | | | | | 150195 | 151627 | |
| 1587 | | | | | | | | | 132737 | 134160 | |
| 1588 | | | | | | | | | 1955978 | 1958455 | |
| 1589 | | | | | | | | | 485658 | 488135 | |
| 1590 | | | | | | | | | 278588 | 281125 | |
| 1591 | | | | | | | | | 83713 | 86250 | |
| 1592 | | | | | | | | | 279549 | 282086 | |
| 1593 | | | | | | | | | 505157 | 507634 | |
| 1594 | | | | | | | | | 505059 | 507536 | |
| 1595 | | | | | | | | | 467434 | 469911 | |
| 1596 | | | | | | | | | 505205 | 507682 | |
| 1597 | | | | | | | | | 467455 | 469932 | |
| 1598 | | | | | | | | | 467255 | 469732 | |
| 1599 | | | | | | | | | 485332 | 487809 | |
| 1600 | | | | | | | | | 3065568 | 3066757 | |
| 1601 | | | | | | | | | 2559672 | 2561088 | |
| 1602 | | | | | | | | | 1235298 | 1236673 | |
| 1603 | | | | | | | | | 1860855 | 1862226 | |
| 1604 | | | | | | | | | 348719 | 350085 | |
| 1605 | | | | | | | | | 251369 | 252744 | |
| 1606 | | | | | | | | | 33638 | 35013 | |
| 1607 | | | | | | | | | 696957 | 698323 | |
| 1608 | | | | | | | | | 90011 | 91494 | |
| 1609 | | | | | | | | | 167083 | 168566 | |
| 1610 | | | | | | | | | 190234 | 191717 | |
| 1611 | | | | | | | | | 421379 | 422862 | |
| 1612 | | | | | | | | | 1485164 | 1486647 | |
| 1613 | | | | | | | | | 41076 | 42559 | |
| 1614 | | | | | | | | | 68604 | 70087 | |
| 1615 | | | | | | | | | 68623 | 70106 | |
| 1616 | | | | | | | | | 115692 | 117175 | |
| 1617 | | | | | | | | | 23055 | 24538 | |
| 1618 | | | | | | | | | 22088 | 23571 | |
| 1619 | | | | | | | | | 195988 | 197471 | |
| 1620 | | | | | | | | | 8031 | 9514 | |
| 1621 | | | | | | | | | 22905 | 24388 | |
| 1622 | | | | | | | | | 655324 | 656726 | |
| 1623 | | | | | | | | | 184 | 1586 | |
| 1624 | | | | | | | | | 378494 | 379896 | |
| 1625 | | | | | | | | | 2737817 | 2739300 | |
| 1626 | | | | | | | | | 188075 | 189558 | |
| 1627 | | | | | | | | | 217136 | 218619 | |
| 1628 | | | | | | | | | 4679 | 6125 | |
| 1629 | | | | | | | | | 618232 | 619597 | |
| 1630 | | | | | | | | | 58857 | 60199 | |
| 1631 | | | | | | | | | 25138 | 26419 | |
| 1632 | | | | | | | | | 878549 | 880212 | |
| 1633 | | | | | | | | | 1017795 | 1019207 | |
| 1634 | | | | | | | | | 970449 | 971790 | |
| 1635 | | | | | | | | | 279792 | 281124 | |
| 1636 | | | | | | | | | 234529 | 235862 | |
| 1637 | | | | | | | | | 43541 | 44877 | |
| 1638 | | | | | | | | | 24580 | 25916 | |
| 1639 | | | | | | | | | 26348 | 27675 | |
| 1640 | | | | | | | | | 4106 | 5433 | |
| 1641 | | | | | | | | | 432088 | 433415 | |
| 1642 | | | | | | | | | 2015511 | 2016838 | |
| 1643 | | | | | | | | | 198483 | 199621 | |
| 1644 | | | | | | | | | 12987 | 14314 | |
| 1645 | | | | | | | | | 10816 | 11954 | |
| 1646 | | | | | | | | | 1 | 738 | |
| 1647 | | | | | | | | | 45207 | 46534 | |
| 1648 | | | | | | | | | 15185 | 16512 | |
| 1649 | | | | | | | | | 11095 | 12233 | |
| 1650 | | | | | | | | | 16291 | 17618 | |
| 1651 | | | | | | | | | 922 | 2380 | |
| 1652 | | | | | | | | | 80649 | 82053 | |
| 1653 | | | | | | | | | 98527 | 99931 | |
| 1654 | | | | | | | | | 80649 | 82053 | |
| 1655 | | | | | | | | | 79320 | 80724 | |
| 1656 | | | | | | | | | 5485 | 6910 | |
| 1657 | | | | | | | | | 2391314 | 2392718 | |
| 1658 | | | | | | | | | 98697 | 100101 | |
| 1659 | | | | | | | | | 1629344 | 1630748 | |
| 1660 | | | | | | | | | 1587489 | 1588893 | |
| 1661 | | | | | | | | | 1758391 | 1759795 | |
| 1662 | | | | | | | | | 1761238 | 1762642 | |
| 1663 | | | | | | | | | 168103 | 169507 | |
| 1664 | | | | | | | | | 98697 | 100101 | |
| 1665 | | | | | | | | | 394942 | 396346 | |
| 1666 | | | | | | | | | 80649 | 82053 | |
| 1667 | | | | | | | | | 63660 | 64995 | |
| 1668 | | | | | | | | | 157063 | 158363 | |
| 1669 | | | | | | | | | 3080200 | 3081662 | |
| 1670 | | | | | | | | | 73291 | 74729 | |
| 1671 | | | | | | | | | 15431 | 16773 | |
| 1672 | | | | | | | | | 367310 | 368729 | |
| 1673 | | | | | | | | | 1719 | 3138 | |
| 1674 | | | | | | | | | 86400 | 87735 | |
| 1675 | | | | | | | | | 17508 | 18843 | |
| 1676 | | | | | | | | | 552979 | 555192 | |
| 1677 | | | | | | | | | 537536 | 538890 | |
| 1678 | | | | | | | | | 50209 | 51614 | |
| 1679 | | | | | | | | | 18293 | 19811 | |
| 1680 | | | | | | | | | 855094 | 856495 | |
| 1681 | | | | | | | | | 790419 | 791775 | |
| 1682 | | | | | | | | | 151715 | 153071 | |
| 1683 | | | | | | | | | 1035215 | 1036571 | |
| 1684 | | | | | | | | | 61907 | 63263 | |
| 1685 | | | | | | | | | 52334 | 53516 | |
| 1686 | | | | | | | | | 51361 | 52543 | |
| 1687 | | | | | | | | | 16395 | 17577 | |
| 1688 | | | | | | | | | 15348 | 16530 | |
| 1689 | | | | | | | | | 774460 | 775816 | |
| 1690 | | | | | | | | | 951413 | 952763 | |
| 1691 | | | | | | | | | 3133822 | 3135172 | |
| 1692 | | | | | | | | | 1666481 | 1667831 | |
| 1693 | | | | | | | | | 1054353 | 1055733 | |
| 1694 | | | | | | | | | 3639547 | 3640897 | |
| 1695 | | | | | | | | | 763489 | 764869 | |
| 1696 | | | | | | | | | 4111 | 5512 | |
| 1697 | | | | | | | | | 3956631 | 3958032 | |
| 1698 | | | | | | | | | 10999 | 12349 | |
| 1699 | | | | | | | | | 51317 | 52718 | |
| 1700 | | | | | | | | | 51317 | 52718 | |
| 1701 | | | | | | | | | 163388 | 164744 | |
| 1702 | | | | | | | | | 207731 | 209087 | |
| 1703 | | | | | | | | | 834005 | 835406 | |
| 1704 | | | | | | | | | 825117 | 826473 | |
| 1705 | | | | | | | | | 848376 | 849726 | |
| 1706 | | | | | | | | | 51424 | 52804 | |
| 1707 | | | | | | | | | 4446 | 5796 | |
| 1708 | | | | | | | | | 4446 | 5796 | |
| 1709 | | | | | | | | | 182936 | 184286 | |
| 1710 | | | | | | | | | 179243 | 180593 | |
| 1711 | | | | | | | | | 4446 | 5796 | |
| 1712 | | | | | | | | | 4446 | 5796 | |
| 1713 | | | | | | | | | 4417 | 5797 | |
| 1714 | | | | | | | | | 179237 | 180587 | |
| 1715 | | | | | | | | | 179433 | 180783 | |
| 1716 | | | | | | | | | 4412 | 5762 | |
| 1717 | | | | | | | | | 2681 | 4031 | |
| 1718 | | | | | | | | | 181925 | 183305 | |
| 1719 | | | | | | | | | 4443 | 5823 | |
| 1720 | | | | | | | | | 51272 | 52652 | |
| 1721 | | | | | | | | | 179390 | 180770 | |
| 1722 | | | | | | | | | 4412 | 5762 | |
| 1723 | | | | | | | | | 179263 | 180613 | |
| 1724 | | | | | | | | | 53137 | 54487 | |
| 1725 | | | | | | | | | 182219 | 183599 | |
| 1726 | | | | | | | | | 347718 | 349068 | |
| 1727 | | | | | | | | | 4443 | 5823 | |
| 1728 | | | | | | | | | 179226 | 180606 | |
| 1729 | | | | | | | | | 179431 | 180781 | |
| 1730 | | | | | | | | | 4446 | 5796 | |
| 1731 | | | | | | | | | 4446 | 5826 | |
| 1732 | | | | | | | | | 181022 | 182402 | |
| 1733 | | | | | | | | | 4446 | 5826 | |
| 1734 | | | | | | | | | 179425 | 180775 | |
| 1735 | | | | | | | | | 181377 | 182727 | |
| 1736 | | | | | | | | | 4443 | 5793 | |
| 1737 | | | | | | | | | 179233 | 180613 | |
| 1738 | | | | | | | | | 4446 | 5826 | |
| 1739 | | | | | | | | | 179233 | 180613 | |
| 1740 | | | | | | | | | 4442 | 5792 | |
| 1741 | | | | | | | | | 201348 | 202728 | |
| 1742 | | | | | | | | | 179317 | 180667 | |
| 1743 | | | | | | | | | 143710 | 145060 | |
| 1744 | | | | | | | | | 56633 | 58013 | |
| 1745 | | | | | | | | | 2681 | 4061 | |
| 1746 | | | | | | | | | 2680 | 4060 | |
| 1747 | | | | | | | | | 837068 | 838424 | |
| 1748 | | | | | | | | | 837352 | 838708 | |
| 1749 | | | | | | | | | 3604904 | 3606260 | |
| 1750 | | | | | | | | | 2690 | 4070 | |
| 1751 | | | | | | | | | 4446 | 5826 | |
| 1752 | | | | | | | | | 179438 | 180788 | |
| 1753 | | | | | | | | | 179224 | 180604 | |
| 1754 | | | | | | | | | 4441 | 5791 | |
| 1755 | | | | | | | | | 179249 | 180599 | |
| 1756 | | | | | | | | | 4446 | 5826 | |
| 1757 | | | | | | | | | 51372 | 52722 | |
| 1758 | | | | | | | | | 4446 | 5826 | |
| 1759 | | | | | | | | | 4425 | 5775 | |
| 1760 | | | | | | | | | 4446 | 5796 | |
| 1761 | | | | | | | | | 4446 | 5826 | |
| 1762 | | | | | | | | | 4446 | 5796 | |
| 1763 | | | | | | | | | 4425 | 5775 | |
| 1764 | | | | | | | | | 179241 | 180591 | |
| 1765 | | | | | | | | | 4412 | 5762 | |
| 1766 | | | | | | | | | 179408 | 180788 | |
| 1767 | | | | | | | | | 4441 | 5821 | |
| 1768 | | | | | | | | | 179422 | 180772 | |
| 1769 | | | | | | | | | 179114 | 180494 | |
| 1770 | | | | | | | | | 179207 | 180587 | |
| 1771 | | | | | | | | | 52653 | 54033 | |
| 1772 | | | | | | | | | 4378 | 5758 | |
| 1773 | | | | | | | | | 4437 | 5787 | |
| 1774 | | | | | | | | | 179117 | 180467 | |
| 1775 | | | | | | | | | 179226 | 180606 | |
| 1776 | | | | | | | | | 179442 | 180792 | |
| 1777 | | | | | | | | | 4414 | 5764 | |
| 1778 | | | | | | | | | 179412 | 180792 | |
| 1779 | | | | | | | | | 4446 | 5826 | |
| 1780 | | | | | | | | | 180295 | 181645 | |
| 1781 | | | | | | | | | 179423 | 180803 | |
| 1782 | | | | | | | | | 51342 | 52722 | |
| 1783 | | | | | | | | | 179412 | 180792 | |
| 1784 | | | | | | | | | 4443 | 5793 | |
| 1785 | | | | | | | | | 347708 | 349088 | |
| 1786 | | | | | | | | | 4433 | 5783 | |
| 1787 | | | | | | | | | 2676 | 4026 | |
| 1788 | | | | | | | | | 2664 | 4044 | |
| 1789 | | | | | | | | | 2671 | 4051 | |
| 1790 | | | | | | | | | 179328 | 180708 | |
| 1791 | | | | | | | | | 2675 | 4055 | |
| 1792 | | | | | | | | | 247106 | 248456 | |
| 1793 | | | | | | | | | 2662 | 4042 | |
| 1794 | | | | | | | | | 137298 | 138678 | |
| 1795 | | | | | | | | | 179122 | 180472 | |
| 1796 | | | | | | | | | 181161 | 182511 | |
| 1797 | | | | | | | | | 24097 | 26600 | |
| 1798 | | | | | | | | | 1774206 | 1776730 | |
| 1799 | | | | | | | | | 2995200 | 2997724 | |
| 1800 | | | | | | | | | 215678 | 217047 | |
| 1801 | | | | | | | | | 11 | 1365 | |
| 1802 | | | | | | | | | 433369 | 434723 | |
| 1803 | | | | | | | | | 116084 | 117447 | |
| 1804 | | | | | | | | | 134 | 1383 | |
| 1805 | | | | | | | | | 614798 | 616228 | |
| 1806 | | | | | | | | | 2913 | 4396 | |
| 1807 | | | | | | | | | 1236162 | 1237611 | |
| 1808 | | | | | | | | | 52273 | 54418 | |
| 1809 | | | | | | | | | 103276 | 104603 | |
| 1810 | | | | | | | | | 103269 | 104596 | |
| 1811 | | | | | | | | | 230894 | 232214 | |
| 1812 | | | | | | | | | 32976 | 34498 | |
| 1813 | | | | | | | | | 142644 | 144066 | |
| 1814 | | | | | | | | | 2712 | 4835 | |
| 1815 | | | | | | | | | 399088 | 400472 | |
| 1816 | | | | | | | | | 239679 | 241054 | |
| 1817 | | | | | | | | | 1893650 | 1894991 | |
| 1818 | | | | | | | | | 392918 | 394286 | |
| 1819 | | | | | | | | | 845508 | 848346 | Additional ThsA homolog EHI61161.1 and EHI61160.1 |
| 1820 | | | | | | | | | 8273 | 9663 | |
| 1821 | | | | | | | | | 5106952 | 5108412 | |
| 1822 | | | | | | | | | 67402 | 68729 | |
| 1823 | | | | | | | | | 385610 | 386991 | |
| 1824 | | | | | | | | | 391196 | 392625 | |
| 1825 | | | | | | | | | 10416 | 11602 | |
| 1826 | | | | | | | | | 1 | 1463 | |
| 1827 | | | | | | | | | 1773192 | 1774570 | |
| 1828 | | | | | | | | | 93771 | 95197 | |
| 1829 | | | | | | | | | 52979 | 54377 | |
| 1830 | | | | | | | | | 169073 | 170436 | |
| 1831 | | | | | | | | | 111454 | 112853 | |
| 1832 | | | | | | | | | 295 | 1674 | |
| 1833 | | | | | | | | | 52811 | 54295 | |
| 1834 | | | | | | | | | 22017 | 23446 | |
| 1835 | | | | | | | | | 46093 | 47494 | |
| 1836 | | | | | | | | | 10780 | 12281 | |
| 1837 | | | | | | | | | 578586 | 580026 | |
| 1838 | | | | | | | | | 18949 | 20411 | |
| 1839 | | | | | | | | | 1720 | 3139 | |
| 1840 | | | | | | | | | 402136 | 403598 | |
| 1841 | | | | | | | | | 17069 | 18543 | |
| 1842 | | | | | | | | | 778829 | 780278 | |
| 1843 | | | | | | | | | 239846 | 241330 | |
| 1844 | | | | | | | | | 8796 | 9991 | |
| 1845 | | | | | | | | | 8027 | 9442 | |
| 1846 | | | | | | | | | 10583 | 11998 | |
| 1847 | | | | | | | | | 10583 | 11998 | |
| 1848 | | | | | | | | | 101221 | 102636 | |
| 1849 | | | | | | | | | 108258 | 109673 | |
| 1850 | | | | | | | | | 73923 | 75338 | |
| 1851 | | | | | | | | | 9557 | 10972 | |
| 1852 | | | | | | | | | 8731 | 10146 | |
| 1853 | | | | | | | | | 10583 | 11998 | |
| 1854 | | | | | | | | | 9409 | 10824 | |
| 1855 | | | | | | | | | 3016 | 4431 | |
| 1856 | | | | | | | | | 9405 | 10820 | |
| 1857 | | | | | | | | | 10583 | 11998 | |
| 1858 | | | | | | | | | 73814 | 75229 | |
| 1859 | | | | | | | | | 10583 | 11998 | |
| 1860 | | | | | | | | | 75727 | 77531 | Additional ThsA homolog ERH59123.1 |
| 1861 | | | | | | | | | 2134919 | 2137041 | |
| 1862 | | | | | | | | | 794273 | 796356 | |
| 1863 | | | | | | | | | 27958 | 29914 | |
| 1864 | | | | | | | | | 58581 | 61165 | |
| 1865 | | | | | | | | | 25655 | 27734 | |
| 1866 | | | | | | | | | 2276956 | 2281580 | |
| 1867 | | | | | | | | | 214397 | 218349 | |
| 1868 | | | | | | | | | 2202763 | 2205300 | |
| 1869 | | | | | | | | | 72963 | 75500 | |
| 1870 | | | | | | | | | 72753 | 75290 | |
| 1871 | | | | | | | | | 335401 | 337938 | |
| 1872 | | | | | | | | | 73026 | 75563 | |
| 1873 | | | | | | | | | 96286 | 98823 | |
| 1874 | | | | | | | | | 78963 | 81500 | |
| 1875 | | | | | | | | | 626450 | 628927 | |
| 1876 | | | | | | | | | 2144499 | 2147036 | |
| 1877 | | | | | | | | | 2144499 | 2147036 | |
| 1878 | | | | | | | | | 857876 | 860413 | |
| 1879 | | | | | | | | | 190277 | 192754 | |
| 1880 | | | | | | | | | 292755 | 295292 | |
| 1881 | | | | | | | | | 332657 | 335194 | |
| 1882 | | | | | | | | | 333878 | 336415 | |
| 1883 | | | | | | | | | 294681 | 297218 | |
| 1884 | | | | | | | | | 115649 | 118186 | |
| 1885 | | | | | | | | | 332547 | 335084 | |
| 1886 | | | | | | | | | 315968 | 318505 | |
| 1887 | | | | | | | | | 299437 | 301974 | |
| 1888 | | | | | | | | | 294697 | 297234 | |
| 1889 | | | | | | | | | 415578 | 418115 | |
| 1890 | | | | | | | | | 299437 | 301974 | |
| 1891 | | | | | | | | | 919475 | 922012 | |
| 1892 | | | | | | | | | 751819 | 754356 | |
| 1893 | | | | | | | | | 414378 | 416915 | |
| 1894 | | | | | | | | | 430384 | 432921 | |
| 1895 | | | | | | | | | 1711734 | 1714271 | |
| 1896 | | | | | | | | | 1827507 | 1830044 | |
| 1897 | | | | | | | | | 807551 | 810088 | |
| 1898 | | | | | | | | | 722094 | 724631 | |
| 1899 | | | | | | | | | 2570589 | 2573126 | |
| 1900 | | | | | | | | | 771268 | 773805 | |
| 1901 | | | | | | | | | 769990 | 772527 | |
| 1902 | | | | | | | | | 1718201 | 1720738 | |
| 1903 | | | | | | | | | 763807 | 766344 | |
| 1904 | | | | | | | | | 745301 | 747838 | |
| 1905 | | | | | | | | | 1739780 | 1742317 | |
| 1906 | | | | | | | | | 1808916 | 1811453 | |
| 1907 | | | | | | | | | 1830255 | 1832792 | |
| 1908 | | | | | | | | | 637302 | 639839 | |
| 1909 | | | | | | | | | 2820435 | 2822972 | |
| 1910 | | | | | | | | | 4138023 | 4140560 | |
| 1911 | | | | | | | | | 952621 | 955158 | |
| 1912 | | | | | | | | | 424893 | 427430 | |
| 1913 | | | | | | | | | 4840540 | 4843077 | |
| 1914 | | | | | | | | | 415549 | 418086 | |
| 1915 | | | | | | | | | 2733635 | 2736172 | |
| 1916 | | | | | | | | | 949017 | 951554 | |
| 1917 | | | | | | | | | 328880 | 331417 | |
| 1918 | | | | | | | | | 390295 | 392832 | |
| 1919 | | | | | | | | | 977582 | 980119 | |
| 1920 | | | | | | | | | 1830964 | 1833501 | |
| 1921 | | | | | | | | | 767420 | 769957 | |
| 1922 | | | | | | | | | 1643560 | 1646097 | |
| 1923 | | | | | | | | | 1378858 | 1381395 | |
| 1924 | | | | | | | | | 755487 | 758023 | |
| 1925 | | | | | | | | | 881168 | 883705 | |
| 1926 | | | | | | | | | 1825551 | 1828088 | |
| 1927 | | | | | | | | | 738544 | 741081 | |
| 1928 | | | | | | | | | 142767 | 145304 | |
| 1929 | | | | | | | | | 964862 | 967399 | |
| 1930 | | | | | | | | | 776235 | 778772 | |
| 1931 | | | | | | | | | 655024 | 657561 | |
| 1932 | | | | | | | | | 1874434 | 1876971 | |
| 1933 | | | | | | | | | 624797 | 627334 | |
| 1934 | | | | | | | | | 128934 | 131471 | |
| 1935 | | | | | | | | | 136721 | 139258 | |
| 1936 | | | | | | | | | 1726846 | 1729383 | |
| 1937 | | | | | | | | | 823627 | 826164 | |
| 1938 | | | | | | | | | 1937935 | 1940472 | |
| 1939 | | | | | | | | | 1944646 | 1947183 | |
| 1940 | | | | | | | | | 993314 | 995851 | |
| 1941 | | | | | | | | | 821085 | 823622 | |
| 1942 | | | | | | | | | 651372 | 653909 | |
| 1943 | | | | | | | | | 958507 | 962380 | |
| 1944 | | | | | | | | | 1037695 | 1040232 | |
| 1945 | | | | | | | | | 2023087 | 2025624 | |
| 1946 | | | | | | | | | 413025 | 415562 | |
| 1947 | | | | | | | | | 925402 | 927939 | |
| 1948 | | | | | | | | | 1824339 | 1826876 | |
| 1949 | | | | | | | | | 1963306 | 1965843 | |
| 1950 | | | | | | | | | 1723817 | 1726354 | |
| 1951 | | | | | | | | | 1061869 | 1064406 | |
| 1952 | | | | | | | | | 455375 | 457912 | |
| 1953 | | | | | | | | | 99107 | 101644 | |
| 1954 | | | | | | | | | 134788 | 137325 | |
| 1955 | | | | | | | | | 415169 | 417706 | |
| 1956 | | | | | | | | | 411378 | 413915 | |
| 1957 | | | | | | | | | 1723198 | 1725735 | |
| 1958 | | | | | | | | | 969440 | 971977 | |
| 1959 | | | | | | | | | 119038 | 121575 | |
| 1960 | | | | | | | | | 779299 | 781836 | |
| 1961 | | | | | | | | | 837980 | 840517 | |
| 1962 | | | | | | | | | 957690 | 960227 | |
| 1963 | | | | | | | | | 647128 | 649665 | |
| 1964 | | | | | | | | | 1304387 | 1306924 | |
| 1965 | | | | | | | | | 118566 | 121103 | |
| 1966 | | | | | | | | | 964548 | 967085 | |
| 1967 | | | | | | | | | 1037220 | 1039757 | |
| 1968 | | | | | | | | | 1765512 | 1768049 | |
| 1969 | | | | | | | | | 959782 | 962319 | |
| 1970 | | | | | | | | | 1631617 | 1634154 | |
| 1971 | | | | | | | | | 955226 | 957763 | |
| 1972 | | | | | | | | | 1051957 | 1054494 | |
| 1973 | | | | | | | | | 404789 | 407326 | |
| 1974 | | | | | | | | | 994327 | 996864 | |
| 1975 | | | | | | | | | 422844 | 425381 | |
| 1976 | | | | | | | | | 1057307 | 1059844 | |
| 1977 | | | | | | | | | 752850 | 755387 | |
| 1978 | | | | | | | | | 435581 | 438118 | |
| 1979 | | | | | | | | | 415671 | 418208 | |
| 1980 | | | | | | | | | 593893 | 596430 | |
| 1981 | | | | | | | | | 2084900 | 2087438 | |
| 1982 | | | | | | | | | 945151 | 947688 | |
| 1983 | | | | | | | | | 699363 | 701900 | |
| 1984 | | | | | | | | | 696094 | 698631 | |
| 1985 | | | | | | | | | 1817777 | 1820314 | |
| 1986 | | | | | | | | | 844786 | 847323 | |
| 1987 | | | | | | | | | 140683 | 143220 | |
| 1988 | | | | | | | | | 849733 | 852270 | |
| 1989 | | | | | | | | | 840414 | 842951 | |
| 1990 | | | | | | | | | 949044 | 951581 | |
| 1991 | | | | | | | | | 415164 | 417701 | |
| 1992 | | | | | | | | | 863928 | 866465 | |
| 1993 | | | | | | | | | 415572 | 418109 | |
| 1994 | | | | | | | | | 747799 | 750336 | |
| 1995 | | | | | | | | | 418408 | 420945 | |
| 1996 | | | | | | | | | 961748 | 964285 | |
| 1997 | | | | | | | | | 419569 | 422106 | |
| 1998 | | | | | | | | | 1015638 | 1018175 | |
| 1999 | | | | | | | | | 889676 | 892213 | |
| 2000 | | | | | | | | | 948468 | 951005 | |
| 2001 | | | | | | | | | 1885183 | 1887720 | |
| 2002 | | | | | | | | | 966038 | 968575 | |
| 2003 | | | | | | | | | 960076 | 962613 | |
| 2004 | | | | | | | | | 122404 | 124941 | |
| 2005 | | | | | | | | | 1681006 | 1683543 | |
| 2006 | | | | | | | | | 993136 | 995673 | |
| 2007 | | | | | | | | | 998786 | 1001323 | |
| 2008 | | | | | | | | | 423592 | 426129 | |
| 2009 | | | | | | | | | 119049 | 121586 | |
| 2010 | | | | | | | | | 967343 | 969880 | |
| 2011 | | | | | | | | | 777273 | 779810 | |
| 2012 | | | | | | | | | 333878 | 336415 | |
| 2013 | | | | | | | | | 135104 | 137641 | |
| 2014 | | | | | | | | | 414973 | 417510 | |
| 2015 | | | | | | | | | 415129 | 417666 | |
| 2016 | | | | | | | | | 953263 | 955800 | |
| 2017 | | | | | | | | | 413367 | 415904 | |
| 2018 | | | | | | | | | 422253 | 424790 | |
| 2019 | | | | | | | | | 414879 | 417416 | |
| 2020 | | | | | | | | | 332578 | 335115 | |
| 2021 | | | | | | | | | 72684 | 74543 | |
| 2022 | | | | | | | | | 991 | 3528 | |
| 2023 | | | | | | | | | 72684 | 75221 | |
| 2024 | | | | | | | | | 204036 | 206573 | |
| 2025 | | | | | | | | | 51535 | 54072 | |
| 2026 | | | | | | | | | 70748 | 73285 | |
| 2027 | | | | | | | | | 166520 | 169057 | |
| 2028 | | | | | | | | | 1268434 | 1270971 | |
| 2029 | | | | | | | | | 327480 | 330017 | |
| 2030 | | | | | | | | | 1970301 | 1972838 | |
| 2031 | | | | | | | | | 6539 | 9076 | |
| 2032 | | | | | | | | | 2178487 | 2181024 | |
| 2033 | | | | | | | | | 3086900 | 3089437 | |
| 2034 | | | | | | | | | 1764944 | 1767481 | |
| 2035 | | | | | | | | | 892818 | 895355 | |
| 2036 | | | | | | | | | 724298 | 726835 | |
| 2037 | | | | | | | | | 2073702 | 2076179 | |
| 2038 | | | | | | | | | 16866 | 19343 | |
| 2039 | | | | | | | | | 11451 | 13928 | |
| 2040 | | | | | | | | | 14738 | 17215 | |
| 2041 | | | | | | | | | 22974 | 25451 | |
| 2042 | | | | | | | | | 163601 | 166078 | |
| 2043 | | | | | | | | | 55002 | 57479 | |
| 2044 | | | | | | | | | 2345 | 4822 | |
| 2045 | | | | | | | | | 1414 | 3891 | |
| 2046 | | | | | | | | | 36221 | 38698 | |
| 2047 | | | | | | | | | 11452 | 13929 | |
| 2048 | | | | | | | | | 11452 | 13929 | |
| 2049 | | | | | | | | | 11452 | 13929 | |
| 2050 | | | | | | | | | 11455 | 13932 | |
| 2051 | | | | | | | | | 11448 | 13925 | |
| 2052 | | | | | | | | | 11452 | 13929 | |
| 2053 | | | | | | | | | 4703 | 7180 | |
| 2054 | | | | | | | | | 11452 | 13929 | |
| 2055 | | | | | | | | | 56836 | 59313 | |
| 2056 | | | | | | | | | 54998 | 57475 | |
| 2057 | | | | | | | | | 11567 | 14044 | |
| 2058 | | | | | | | | | 18041 | 20518 | |
| 2059 | | | | | | | | | 11452 | 13929 | |
| 2060 | | | | | | | | | 36219 | 38696 | |
| 2061 | | | | | | | | | 11452 | 13929 | |
| 2062 | | | | | | | | | 11450 | 13927 | |
| 2063 | | | | | | | | | 120924 | 123401 | |
| 2064 | | | | | | | | | 36218 | 38695 | |
| 2065 | | | | | | | | | 11448 | 13925 | |
| 2066 | | | | | | | | | 11452 | 13929 | |
| 2067 | | | | | | | | | 11452 | 13929 | |
| 2068 | | | | | | | | | 12006 | 14543 | |
| 2069 | | | | | | | | | 117483 | 119960 | |
| 2070 | | | | | | | | | 6719 | 9196 | |
| 2071 | | | | | | | | | 9656 | 12133 | |
| 2072 | | | | | | | | | 94488 | 96965 | |
| 2073 | | | | | | | | | 2528497 | 2530974 | |
| 2074 | | | | | | | | | 1447 | 5265 | |
| 2075 | | | | | | | | | 307506 | 309710 | |
| 2076 | | | | | | | | | 445336 | 447510 | |
| 2077 | | | | | | | | | 197601 | 199542 | |
| 2078 | | | | | | | | | 1343346 | 1345843 | |
| 2079 | | | | | | | | | 1895 | 4396 | |
| 2080 | | | | | | | | | 4806 | 7307 | |
| 2081 | | | | | | | | | 19162 | 21686 | |
| 2082 | | | | | | | | | 185651 | 188127 | |
| 2083 | | | | | | | | | 166065 | 168541 | |
| 2084 | | | | | | | | | 1030001 | 1032525 | |
| 2085 | | | | | | | | | 158687 | 161211 | |
| 2086 | | | | | | | | | 664126 | 666650 | |
| 2087 | | | | | | | | | 1383218 | 1385739 | |
| 2088 | | | | | | | | | 247534 | 250011 | |
| 2089 | | | | | | | | | 19324 | 21800 | |
| 2090 | | | | | | | | | 3794 | 6270 | |
| 2091 | | | | | | | | | 205986 | 208462 | |
| 2092 | | | | | | | | | 163830 | 166306 | |
| 2093 | | | | | | | | | 17595 | 20119 | |
| 2094 | | | | | | | | | 127282 | 129675 | |
| 2095 | | | | | | | | | 140059 | 142319 | |
| 2096 | | | | | | | | | 607545 | 609474 | |
| 2097 | | | | | | | | | 17857 | 20090 | |
| 2098 | | | | | | | | | 811700 | 814237 | |
| 2099 | | | | | | | | | 571 | 3852 | Additional ThsA homolog CDE34944.1 |
| 2100 | | | | | | | | | 1837917 | 1847352 | |

The TIR domain gene, thsB, has a strong tendency (35%) to appear in multiple copies clustered around the thsA gene (FIG. 5A). Such duplication is typical to specificity-conferring genes in defense systems (such as the S subunit in type I R-M systems), where duplication followed by diversification serves for multiple specificities of the system. It is therefore possible that the TIR domain gene is responsible for identification of specific phage patterns, with multiple TIR domain genes serving for recognition of different phage components. Under this hypothesis, it is tempting to suggest that Thoeris is the prokaryotic ancestral form of pathogen-associated molecular pattern (PAMP) receptors.

A recent study showed that TIR domains can have enzymatic NAD+ hydrolase activities. In animal neurons, NAD+ hydrolase activity of the SARM1 TIR domain-containing gene leads to NAD+ depletion and generation of ADP-ribose and cyclic ADP-ribose that form signaling molecules regulating axonal degeneration. An E99A point mutation in the B. amyloliquefaciens Y2 KwaB protein, which aligns with the catalytic residue in the SARM1 NAD-cleaving TIR domain (FIG. 5E) abolished the protective activity of Thoeris (FIG. 5C). Moreover, point mutations in the ThsA NAD binding site predicted to abolish NAD binding also resulted in system inactivation (ThsA:N112A and ThsA:D100A/N115A for the B. cereus and B. amyloliquefaciens systems, respectively). These results mark NAD+ binding and hydrolysis as essential for the anti-phage activity of the Thoeris system.

### Example 6

### Genomic Identification and Analysis of the Druantia System

Objective:

Identify the candidate defense system based on the enrichment of the anchor COG1205.

Methods:

As above

Results:

Another system worthwhile of a brief discussion is the Druantia system (named after a deity from the Gallic mythology). This system is characterized by a very large protein (˜1800-2100aa) containing a DUF1998 (a domain of unknown function), as well as a helicase signature and a Walker A/B motif suggestive of ATP utilization. This polypeptide, named DruE, is encoded by a very large gene, named druE.

The druE gene is typically preceded by a set of highly variable genes with no recognizable domains or function prediction—either 3 genes sized 350-600aa each (Type I; druB, druC, and drub), or two genes sized 700aa-900aa (Type II; druX, druY), or a single large gene of 1000-1200aa (Type III; druF) (FIGS. 6A and 6B). The system is in some cases also preceded by a gene druA, annotated as DUF4338, comprising yet another domain of unknown function; and Type II systems are also associated with a cytosine methylase (FIG. 6A). In at least one instance, druA was downstream (3′) of the druE gene. (FIG. 6A—second embodiment of a Type I Druantia system)

A Type I system (FIG. 6B) cloned from E. coli UMEA 4076-1 into E. coli MG1655 rendered the engineered strain resistant against 4 of the 6 phages tested, and by serially deleting each of the 4 genes in this system, it was verified that all 4 are essential for its activity (FIG. 6C). Notably, DUF1998-containing genes are among the components of the recently reported DISARM and Dpd defense systems, where their function is also unknown. The sheer size of the Druantia system (12 kb of genomic DNA) was unexpected and surprising, suggesting a complex function, and the near-complete absence of recognizable domains in its encoded polypeptides is suggestive of a new mode of defense not shared by prokaryotic defense systems whose mechanism are currently understood.

Elements of the Druantia system (Types I, II, and III, are presented in Table 10.

TABLE 10
The Druantia Defense System and Components Thereof
This table is presented in landscape orientation. Due to the number of columns, the table
has been split into parts. Part A includes the first set of columns and therefore should
be place to the left of subsequent parts (Part B and where relevant Part C). To avoid any
doubt, the rows of the table in each part line up once the tables are placed together.
(Part A)

| | | | | | | | | | I |
|---|---|---|---|---|---|---|---|---|---|
| | A | B | C | D | E | F | G | H | #genes |
| 1 | assembly_accession | taxid | species_taxid | organism_name | infraspecific_name | phylum | genomic_accession | Type | identified |
| 2 | GCA_001372455.1 | 1505 | 1505 | [Clostridium] sordellii | R26833 | Firmicutes | CELG01000003.1 | I | 4 |
| 3 | GCA_001516615.1 | 1765683 | 1765683 | Acidibacillus ferrooxidans | ITV01 | Firmicutes | LPVJ01000027.1 | I | 5 |
| 4 | GCA_000367505.1 | 1315967 | 33934 | Anoxybacillus flavithermus NBRC 109594 | NBRC 109594 | Firmicutes | BARH01000005.1 | I | 4 |
| 5 | GCA_001425485.1 | 1736375 | 1736375 | Aureimonas sp. Leaf427 | Leaf427 | Proteobacteria | LMQW01000021.1 | I | 3 |
| 6 | GCA_001425525.1 | 1736384 | 1736384 | Aureimonas sp. Leaf460 | Leaf460 | Proteobacteria | LMQY01000066.1 | I | 3 |
| 7 | GCA_001043655.1 | 1396 | 1396 | Bacillus cereus | LK9 | Firmicutes | LDUP01000008.1 | I | 4 |
| 8 | GCA_000291115.1 | 1053212 | 1396 | Bacillus cereus HuB5-5 | HuB5-5 | Firmicutes | JH792120.1 | I | 4 |
| 9 | GCA_001048695.1 | 1499688 | 1499688 | Bacillus sp. LF1 | LF1 | Firmicutes | CVRB01000003.1 | I | 4 |
| 10 | GCA_000708005.1 | 1454376 | 28026 | Bifidobacterium pseudocatenulatum IPLA36007 | IPLA36007 | Actinobacteria | JEOD01000003.1 | I | 3 |
| 11 | GCA_000513015.1 | 1161409 | 1161409 | Bifidobacterium sp. MSTE12 | MSTE12 | Actinobacteria | AZYA01000034.1 | I | 3 |
| 12 | GCA_001402875.1 | 1079 | 1079 | Blastochloris viridis | ATCC 19567 | Proteobacteria | CP012946.1 | I | 3 |
| 13 | GCA_001459775.1 | 1079 | 1079 | Blastochloris viridis | | Proteobacteria | LN907867.1 | I | 3 |
| 14 | GCA_000757095.1 | 28450 | 28450 | Burkholderia pseudomallei | BDE | Proteobacteria | KN150937.1 | I | 4 |
| 15 | GCA_000774465.1 | 1434201 | 28450 | Burkholderia pseudomallei MSHR4299 | MSHR4299 | Proteobacteria | KN323020.1 | I | 3 |
| 16 | GCA_000152345.1 | 331978 | 28450 | Burkholderia pseudomallei Pasteur 52237 | Pasteur 52237 | Proteobacteria | CH899741.1 | I | 3 |
| 17 | GCA_000757035.2 | 331978 | 28450 | Burkholderia pseudomallei Pasteur 52237 | Pasteur 52237 | Proteobacteria | CP009899.1 | I | 4 |
| 18 | GCA_000808035.2 | 57975 | 57975 | Burkholderia thailandensis | 2003015869 | Proteobacteria | CP008914.2 | I | 3 |
| 19 | GCA_001527035.1 | 101571 | 101571 | Burkholderia ubonensis | MSMB1607WGS | Proteobacteria | LPAR01000085.1 | I | 4 |
| 20 | GCA_001528155.1 | 101571 | 101571 | Burkholderia ubonensis | MSMB1633WGS | Proteobacteria | LPAU01000139.1 | I | 3 |
| 21 | GCA_001051235.2 | 1400863 | 1400862 | Candidatus Competibacter denitrificans Run_A_D11 | Run_A_D11 | Proteobacteria | CBTJ020000108.1 | I | 3 |
| 22 | GCA_001292585.1 | 1509431 | 1509431 | Candidatus Magnetomorum sp. HK-1 | | Proteobacteria | JPDT01002867.1 | I | 4 |
| 23 | GCA_000785145.1 | 1003181 | 1003181 | Candidatus Thiomargarita nelsonii | | Proteobacteria | JSZA01001556.1 | I | 3 |
| 24 | GCA_000952105.1 | 536 | 536 | Chromobacterium violaceum | L_1B5_1 | Proteobacteria | JYGI01000049.1 | I | 5 |
| 25 | GCA_000577895.1 | 1216932 | 1216932 | Clostridium bornimense | type strain: M2/40 | Firmicutes | HG917869.1 | I | 4 |
| 26 | GCA_000724245.2 | 1443123 | 1542 | Clostridium novyi B str. ATCC 27606 | ATCC 27606 | Firmicutes | JENW01000064.1 | I | 3 |
| 27 | GCA_000190355.1 | 556261 | 556261 | Clostridium sp. D5 | D5 | Firmicutes | GL870812.1 | I | 3 |
| 28 | GCA_000021925.1 | 272564 | 49338 | Desulfitobacterium hafniense DCB-2 | DCB-2 | Firmicutes | CP001336.1 | I | 3 |
| 29 | GCA_000024205.1 | 485916 | 58138 | Desulfotomaculum acetoxidans DSM 771 | DSM 771 | Firmicutes | CP001720.1 | I | 3 |
| 30 | GCA_001545095.1 | 1514105 | 1514105 | Erysipelothrix sp. LV19 | LV19 | Firmicutes | CP013213.1 | I | 3 |
| 31 | GCA_000797815.1 | 562 | 562 | Escherichia coli | CVM N41556PS | Proteobacteria | JUBQ01000038.1 | I | 5 |
| 32 | GCA_000797835.1 | 562 | 562 | Escherichia coli | CVM N33804PS | Proteobacteria | JUDE01000009.1 | I | 5 |
| 33 | GCA_000797855.1 | 562 | 562 | Escherichia coli | CVM N33806PS | Proteobacteria | JUDD01000028.1 | I | 3 |
| 34 | GCA_000798155.1 | 562 | 562 | Escherichia coli | CVM N34077PS | Proteobacteria | JUCZ01000011.1 | I | 5 |
| 35 | GCA_000798415.1 | 562 | 562 | Escherichia coli | CVM N34557PS | Proteobacteria | JUCU01000008.1 | I | 4 |
| 36 | GCA_001077875.1 | 562 | 562 | Escherichia coli | KIC-2 | Proteobacteria | LFJZ01000059.1 | I | 5 |
| 37 | GCA_001420165.1 | 562 | 562 | Escherichia coli | 449 | Proteobacteria | LIVL01000002.1 | I | 5 |
| 38 | GCA_001442495.1 | 562 | 562 | Escherichia coli | YD786 | Proteobacteria | CP013112.1 | I | 5 |
| 39 | GCA_001463535.1 | 562 | 562 | Escherichia coli | 50673720 | Proteobacteria | LNHW01000058.1 | I | 5 |
| 40 | GCA_001576545.1 | 562 | 562 | Escherichia coli | G76 | Proteobacteria | LOPR01000035.1 | I | 5 |
| 41 | GCA_000194295.2 | 869676 | 562 | Escherichia coli 97.0264 | 97.0264 | Proteobacteria | AEZP02000004.1 | I | 5 |
| 42 | GCA_000458275.1 | 1281086 | 562 | Escherichia coli HVH 153 (3-9344314) | HVH 153 (3-9344314) | Proteobacteria | KE700289.1 | I | 5 |
| 43 | GCA_000456745.1 | 1280996 | 562 | Escherichia coli HVH 46 (4-2758776) | HVH 46 (4-2758776) | Proteobacteria | KE699287.1 | I | 5 |
| 44 | GCA_000456745.1 | 1280996 | 562 | Escherichia coli HVH 46 (4-2758776) | HVH 46 (4-2758776) | Proteobacteria | KE699283.1 | I | 5 |
| 45 | GCA_000607285.1 | 1409786 | 562 | Escherichia coli K02 | K02 | Proteobacteria | KK097723.1 | I | 5 |
| 46 | GCA_000459655.1 | 1281158 | 562 | Escherichia coli KOEGE 33 (68a) | KOEGE 33 (68a) | Proteobacteria | KE701247.1 | I | 5 |
| 47 | GCA_000350745.1 | 1169334 | 562 | Escherichia coli KTE15 | KTE15 | Proteobacteria | KB732282.1 | I | 5 |
| 48 | GCA_000352905.1 | 1181729 | 562 | Escherichia coli KTE183 | KTE183 | Proteobacteria | KB733173.1 | I | 5 |
| 49 | GCA_000407945.1 | 1169352 | 562 | Escherichia coli KTE34 | KTE34 | Proteobacteria | KE136741.1 | I | 5 |
| 50 | GCA_000407745.1 | 1169320 | 562 | Escherichia coli KTE35 | KTE35 | Proteobacteria | KE136604.1 | I | 5 |
| 51 | GCA_000351665.1 | 1182679 | 562 | Escherichia coli KTE72 | KTE72 | Proteobacteria | KB733389.1 | I | 5 |
| 52 | GCA_000351805.1 | 1182691 | 562 | Escherichia coli KTE86 | KTE86 | Proteobacteria | KB732864.1 | I | 5 |
| 53 | GCA_000326625.1 | 1169366 | 562 | Escherichia coli KTE88 | KTE88 | Proteobacteria | ANYV01000069.1 | I | 5 |
| 54 | GCA_000408505.1 | 1182693 | 562 | Escherichia coli KTE89 | KTE89 | Proteobacteria | KE137143.1 | I | 5 |
| 55 | GCA_000351845.1 | 1182696 | 562 | Escherichia coli KTE93 | KTE93 | Proteobacteria | KB732902.1 | I | 5 |
| 56 | GCA_000576655.1 | 1314835 | 562 | Escherichia coli MP1 | MP1 | Proteobacteria | JEMI01000044.1 | I | 5 |
| 57 | GCA_000179175.1 | 679207 | 562 | Escherichia coli MS 107-1 | MS 107-1 | Proteobacteria | ADWV01000005.1 | I | 5 |
| 58 | GCA_000179095.1 | 796392 | 562 | Escherichia coli MS 79-10 | MS 79-10 | Proteobacteria | ADWR01000035.1 | I | 3 |
| 59 | GCA_000364305.1 | 1235805 | 562 | Escherichia coli SWW33 | SWW33 | Proteobacteria | KB822527.1 | I | 5 |
| 60 | GCA_000176635.2 | 656443 | 562 | Escherichia coli TA271 | TA271 | Proteobacteria | GL884297.1 | I | 4 |
| 61 | GCA_000460495.1 | 1281201 | 562 | Escherichia coli UMEA 3163-1 | UMEA 3163-1 | Proteobacteria | KE701783.1 | I | 5 |
| 62 | GCA_000461335.1 | 1281248 | 562 | Escherichia coli UMEA 3391-1 | UMEA 3391-1 | Proteobacteria | KE702395.1 | I | 5 |
| 63 | GCA_000461855.1 | 1281278 | 562 | Escherichia coli UMEA 4076-1 | UMEA 4076-1 | Proteobacteria | KE702725.1 | I | 5 |
| 64 | GCA_000434955.1 | 1262994 | 1262994 | Firmicutes bacterium CAG:65 | | Firmicutes | FR882409.1 | I | 4 |
| 65 | GCA_000750005.1 | 1430331 | 1430331 | Geobacillus icigianus | G1w1 | Firmicutes | JPYA01000201.1 | I | 4 |
| 66 | GCA_001587475.1 | 1586290 | 1586290 | Geobacillus sp. B4113_201601 | B4113_201601 | Firmicutes | LQYX01000099.1 | I | 4 |
| 67 | GCA_001025095.1 | 1659191 | 1659191 | Geobacillus sp. T6 | T6 | Firmicutes | LDNZ01000120.1 | I | 3 |
| 68 | GCA_000174795.2 | 550542 | 550542 | Geobacillus sp. Y412MC52 | Y412MC52 | Firmicutes | CP002442.1 | I | 4 |
| 69 | GCA_000024705.1 | 544556 | 544556 | Geobacillus sp. Y412MC61 | Y412MC61 | Firmicutes | CP001794.1 | I | 4 |
| 70 | GCA_000743495.1 | 1422 | 1422 | Geobacillus stearothermophilus | 22 | Firmicutes | JQCS01000005.1 | I | 4 |
| 71 | GCA_000749985.1 | 1422 | 1422 | Geobacillus stearothermophilus | 53 | Firmicutes | JPYV01000046.1 | I | 4 |
| 72 | GCA_000154705.2 | 411684 | 244596 | Hoeflea phototrophica DFL-43 | DFL-43 | Proteobacteria | CM002917.1 | I | 3 |
| 73 | GCA_001428625.1 | 1736490 | 1736490 | Hydrogenophaga sp. Root209 | Root209 | Proteobacteria | LMIE01000033.1 | I | 3 |
| 74 | GCA_000230975.3 | 670307 | 53399 | Hyphomicrobium denitrificans 1NES1 | 1NES1 | Proteobacteria | CP005587.1 | I | 3 |
| 75 | GCA_001078195.1 | 883119 | 571 | Klebsiella oxytoca 10-5244 | 10-5244 | Proteobacteria | KQ235774.1 | I | 5 |
| 76 | GCA_000492915.1 | 1328370 | 573 | Klebsiella pneumoniae MGH 32 | MGH 32 | Proteobacteria | KI535619.1 | I | 4 |
| 77 | GCA_000018685.1 | 357809 | 66219 | Lachnoclostridium phytofermentans ISDg | ISDg | Firmicutes | CP000885.1 | I | 3 |
| 78 | GCA_000403475.2 | 1235799 | 1235799 | Lachnospiraceae bacterium 3-2 | 3-2 | Firmicutes | KE159795.1 | I | 4 |
| 79 | GCA_000170755.1 | 313628 | 256847 | Lentisphaera araneosa HTCC2155 | HTCC2155 | Lentisphaerae | ABCK01000024.1 | I | 3 |
| 80 | GCA_000502535.1 | 1287273 | 1287273 | Mesorhizobium sp. LNJC391B00 | LNJC391B00 | Proteobacteria | AYWI01000025.1 | I | 3 |
| 81 | GCA_001508455.1 | 1641382 | 1641382 | Methanocalculus sp. 52_23 | | Euryarchaeota | LGGJ01000025.1 | I | 4 |
| 82 | GCA_000243255.1 | 937775 | 2315 | Methanoplanus limicola DSM 2279 | DSM 2279 | Euryarchaeota | CM001436.1 | I | 4 |
| 83 | GCA_000979685.1 | 2209 | 2209 | Methanosarcina mazei | 3.H.A.2.4 | Euryarchaeota | JJPP01000022.1 | I | 4 |
| 84 | GCA_000455825.1 | 1333857 | 300020 | Microbacterium maritypicum MF109 | MF109 | Actinobacteria | ATAO01000002.1 | I | 3 |
| 85 | GCA_001314225.1 | 1714373 | 1714373 | Microbacterium sp. No. 7 | No. 7 | Actinobacteria | CP012697.1 | I | 3 |
| 86 | GCA_000509385.1 | 1344956 | 1344956 | Microbacteriumsp. TS-1 | TS-1 | Actinobacteria | BASQ01000001.1 | I | 3 |
| 87 | GCA_000143085.1 | 105559 | 473531 | Nitrosococcus watsonii C-113 | C-113 | Proteobacteria | CP002087.1 | I | 3 |
| 88 | GCA_001044495.1 | 1628279 | 1628279 | Nitrospina sp. SCGC_AAA799_C22 | | Nitrospinae | JZKJ01000047.1 | I | 3 |
| 89 | GCA_000225995.2 | 1003237 | 28077 | Nitrospirillum amazonense Y2 | Y2 | Proteobacteria | AFBX01000767.1 | I | 3 |
| 90 | GCA_000442555.1 | 1117108 | 44250 | Paenibacillus alvei TS-15 | TS-15 | Firmicutes | ATMT01000002.1 | I | 4 |
| 91 | GCA_000024685.1 | 481743 | 481743 | Paenibacillus sp. Y412MC10 | Y412MC10 | Firmicutes | CP001793.1 | I | 4 |
| 92 | GCA_000803315.1 | 1507808 | 554 | Pectobacterium carotovorum subsp. actinidiae | KKH3 | Proteobacteria | JRMH01000001.1 | I | 5 |
| 93 | GCA_000754765.1 | 78398 | 554 | Pectobacterium carotovorum subsp. odoriferum | NCPPB 3839 | Proteobacteria | JQOG01000013.1 | I | 3 |
| 94 | GCA_000271665.2 | 1192197 | 365349 | Pelosinus fermentans JBW45 | JBW45 | Firmicutes | CP010978.1 | I | 4 |
| 95 | GCA_001298735.1 | 1677858 | 1677858 | Propionispora sp. Iso2/2 | | Firmicutes | CYSP01000005.1 | I | 4 |
| 96 | GCA_001450755.1 | 287 | 287 | Pseudomonas aeruginosa | WH-SGI-V-07239 | Proteobacteria | LLNK01000035.1 | I | 4 |
| 97 | GCA_001451905.1 | 287 | 287 | Pseudomonas aeruginosa | WH-SGI-V-07508 | Proteobacteria | LLQO01000185.1 | I | 3 |
| 98 | GCA_001452525.1 | 287 | 287 | Pseudomonas aeruginosa | WH-SGI-V-07690 | Proteobacteria | LLSK01000268.1 | I | 4 |
| 99 | GCA_001548135.1 | 287 | 287 | Pseudomonas aeruginosa | 8380 | Proteobacteria | AP014839.2 | I | 5 |
| 100 | GCA_000647595.1 | 1339263 | 287 | Pseudomonas aeruginosa 148 | 148 | Proteobacteria | ATAJ01000067.1 | I | 4 |
| 101 | GCA_000359505.1 | 1280938 | 287 | Pseudomonas aeruginosa B136-33 | B136-33 | Proteobacteria | CP004061.1 | I | 3 |
| 102 | GCA_000480905.1 | 1402553 | 287 | Pseudomonas aeruginosa BL12 | BL12 | Proteobacteria | KI518868.1 | I | 5 |
| 103 | GCA_000629605.1 | 1418235 | 287 | Pseudomonas aeruginosa BWH049 | BWH049 | Proteobacteria | KK213281.1 | I | 5 |
| 104 | GCA_000481145.1 | 1402528 | 287 | Pseudomonas aeruginosa BWHPSA028 | BWHPSA028 | Proteobacteria | KI518960.1 | I | 5 |
| 105 | GCA_000455705.1 | 1333855 | 287 | Pseudomonas aeruginosa WC55 | WC55 | Proteobacteria | ATAQ01000308.1 | I | 4 |
| 106 | GCA_000934565.1 | 294 | 294 | Pseudomonas fluorescens | PCL1751 | Proteobacteria | CP010896.1 | I | 5 |
| 107 | GCA_000497385.1 | 1399145 | 303 | Pseudomonas putida S610 | S610 | Proteobacteria | AYJQ01000058.1 | I | 5 |
| 108 | GCA_000367825.1 | 1227924 | 303 | Pseudomonas putida TRO1 | TRO1 | Proteobacteria | APBQ01000198.1 | I | 5 |
| 109 | GCA_000812405.1 | 1582493 | 1582493 | Pseudomonas sp. PI1 | PI1 | Proteobacteria | JWMC01000074.1 | I | 3 |
| 110 | GCA_000737225.1 | 317 | 317 | Pseudomonas syringae | UB0390 | Proteobacteria | JPQV01000021.1 | I | 5 |
| 111 | GCA_001587135.1 | 305 | 305 | Ralstonia solanacearum | UW163 | Proteobacteria | CP012941.1 | I | 5 |
| 112 | GCA_001423045.1 | 1736327 | 1736327 | Rathayibacter sp. Leaf296 | Leaf296 | Actinobacteria | LMNR01000001.1 | I | 3 |
| 113 | GCA_001425345.1 | 1736357 | 1736357 | Rhizobium sp. Leaf383 | Leaf383 | Proteobacteria | LMQD01000012.1 | I | 3 |
| 114 | GCA_001424065.1 | 1736358 | 1736358 | Rhizobium sp. Leaf384 | Leaf384 | Proteobacteria | LMQE01000005.1 | I | 3 |
| 115 | GCA_000982715.1 | 191292 | 191292 | Rhodococcus aetherivorans | IcdP1 | Actinobacteria | CP011341.1 | I | 3 |
| 116 | GCA_000341815.1 | 1278077 | 334542 | Rhodococcus qingshengii BKS 20-40 | BKS 20-40 | Actinobacteria | AODN01000014.1 | I | 3 |
| 117 | GCA_000824945.1 | 1830 | 1830 | Rhodococcus ruber | | Actinobacteria | CCSD01000049.1 | I | 4 |
| 118 | GCA_000508185.1 | 1207055 | 1207055 | Sphingobium sp. C100 | C100 | Proteobacteria | AYOY01000167.1 | I | 3 |
| 119 | GCA_000158915.1 | 457427 | 998084 | Streptomyces himastatinicus ATCC 53653 | ATCC 53653 | Actinobacteria | GG657754.1 | I | 3 |
| 120 | GCA_000227705.3 | 717605 | 377615 | Thermobacillus composti KWC4 | KWC4 | Firmicutes | CP003255.1 | I | 3 |
| 121 | GCA_000176455.1 | 675809 | 666 | Vibrio cholerae CT 5369-93 | CT 5369-93 | Proteobacteria | ADAL01000217.1 | I | 5 |
| 122 | GCA_000746665.1 | 672 | 672 | Vibrio vulnificus | 93U204 | Proteobacteria | CP009261.1 | I | 5 |
| 123 | GCA_001116865.1 | 631 | 631 | Yersinia intermedia | 58735 | Proteobacteria | CQER01000007.1 | I | 5 |
| 124 | GCA_001299035.1 | 1806494 | 1806494 | Achromobacter sp. 2789STDY5608612 | 2789STDY5608612 | Proteobacteria | CYTE01000002.1 | II | 4 |
| 125 | GCA_001509495.1 | 135947 | 135946 | Actinoplanes awajinensis subsp. mycoplanecinus | NRRL B-16712 | Actinobacteria | LLZH01000223.1 | II | 3 |
| 126 | GCA_000389965.1 | 649831 | 649831 | Actinoplanes sp. N902-109 | N902-109 | Actinobacteria | CP005929.1 | II | 3 |
| 127 | GCA_000219665.2 | 1050720 | 358 | Agrobacterium tumefaciens F2 | F2 | Proteobacteria | AFSD01000008.1 | II | 4 |
| 128 | GCA_000018325.1 | 350688 | 461876 | Alkaliphilus oremlandii OhILAs | OhILAs | Firmicutes | CP000853.1 | II | 3 |
| 129 | GCA_001443605.1 | 1307839 | 1307839 | bacterium L21-Spi-D4 | L21-Spi-D4 | Bacteroidetes | CP013118.1 | II | 3 |
| 130 | GCA_000244935.1 | 319003 | 319003 | Bradyrhizobium sp. WSM1253 | WSM1253 | Proteobacteria | JH600073.1 | II | 4 |
| 131 | GCA_000144605.1 | 633149 | 74313 | Brevundimonas subvibrioides ATCC 15264 | ATCC 15264 | Proteobacteria | CP002102.1 | II | 4 |
| 132 | GCA_000182255.1 | 513051 | 87883 | Burkholderia multivorans CGD1 | CGD1 | Proteobacteria | ACFB01000002.1 | II | 3 |
| 133 | GCA_001532035.1 | 1207504 | 1207504 | Burkholderia pseudomultivorans | MSMB368WGS | Proteobacteria | LPJR01000034.1 | II | 4 |
| 134 | GCA_001424345.1 | 1736287 | 1736287 | Burkholderia sp. Leaf177 | Leaf177 | Proteobacteria | LMPF01000022.1 | II | 4 |
| 135 | GCA_001526735.1 | 101571 | 101571 | Burkholderia ubonensis | MSMB1601WGS | Proteobacteria | LPAL01000127.1 | II | 3 |
| 136 | GCA_001526945.1 | 101571 | 101571 | Burkholderia ubonensis | MSMB1791WGS | Proteobacteria | LPAY01000146.1 | II | 3 |
| 137 | GCA_001529895.1 | 101571 | 101571 | Burkholderia ubonensis | MSMB1216WGS | Proteobacteria | LPEG01000082.1 | II | 4 |
| 138 | GCA_001531945.1 | 101571 | 101571 | Burkholderia ubonensis | MSMB609WGS | Proteobacteria | LPKF01000002.1 | II | 4 |
| 139 | GCA_001534345.1 | 101571 | 101571 | Burkholderia ubonensis | MSMB1466WGS | Proteobacteria | LPMV01000059.1 | II | 3 |
| 140 | GCA_001426725.1 | 1736459 | 1736459 | Cellulomonas sp. Root137 | Root137 | Actinobacteria | LMFG01000005.1 | II | 3 |
| 141 | GCA_000016085.1 | 290318 | 1094 | Chlorobium phaeovibrioides DSM 265 | DSM 265 | Chlorobi | CP000607.1 | II | 3 |
| 142 | GCA_001563495.1 | 267125 | 267125 | Chryseobacterium kwangjuense | KJ1R5 | Bacteroidetes | LPUR01000001.1 | II | 3 |
| 143 | GCA_001281005.1 | 546 | 546 | Citrobacter freundii | P10159 | Proteobacteria | CP012554.1 | II | 4 |
| 144 | GCA_000158355.2 | 469595 | 469595 | Citrobacter sp. 30_2 | 30_2 | Proteobacteria | KI391991.1 | II | 4 |
| 145 | GCA_000759755.1 | 1114921 | 67827 | Citrobacter werkmanii NBRC 105721 | NBRC 105721 | Proteobacteria | BBMW01000007.1 | II | 4 |
| 146 | GCA_001515955.1 | 1734399 | 1734399 | Clostridia bacterium BRH_c25 | | Firmicutes | LOES01000090.1 | II | 3 |
| 147 | GCA_001584185.1 | 279113 | 279113 | Collimonas pratensis | Ter91 | Proteobacteria | CP013234.1 | II | 4 |
| 148 | GCA_000725405.1 | 1274 | 1274 | Dermacoccus nishinomiyaensis | M25 | Actinobacteria | CP008889.1 | II | 3 |
| 149 | GCA_000023565.1 | 561229 | 556 | Dickeya chrysanthemi Ech1591 | Ech1591 | Proteobacteria | CP001655.1 | II | 4 |
| 150 | GCA_001011625.1 | 548 | 548 | Enterobacter aerogenes | GN02761 | Proteobacteria | LDBO01000027.1 | II | 4 |
| 151 | GCA_001518055.1 | 548 | 548 | Enterobacter aerogenes | GN02761 | Proteobacteria | LRCC01000127.1 | II | 4 |
| 152 | GCA_001518435.1 | 548 | 548 | Enterobacter aerogenes | GN05782 | Proteobacteria | LRCY01000101.1 | II | 4 |
| 153 | GCA_001011955.1 | 61645 | 61645 | Enterobacter asburiae | GN02208 | Proteobacteria | LDCK01000014.1 | II | 3 |
| 154 | GCA_000958325.1 | 550 | 550 | Enterobacter cloacae | 44517 | Proteobacteria | JZXX01000013.1 | II | 4 |
| 155 | GCA_000958345.1 | 550 | 550 | Enterobacter cloacae | 44524 | Proteobacteria | JZXW01000013.1 | II | 4 |
| 156 | GCA_000958385.1 | 550 | 550 | Enterobacter cloacae | 44541 | Proteobacteria | JZXU01000010.1 | II | 4 |
| 157 | GCA_000965895.1 | 550 | 550 | Enterobacter cloacae | CIDEIMsCOL13 | Proteobacteria | JZKX01000010.1 | II | 4 |
| 158 | GCA_000965925.1 | 550 | 550 | Enterobacter cloacae | CIDEIMsCOL16 | Proteobacteria | JZLA01000009.1 | II | 4 |
| 159 | GCA_000965975.1 | 550 | 550 | Enterobacter cloacae | CIDEIMsCOL18 | Proteobacteria | JZLC01000004.1 | II | 4 |
| 160 | GCA_000965985.1 | 550 | 550 | Enterobacter cloacae | CIDEIMsCOL19 | Proteobacteria | JZLD01000016.1 | II | 4 |
| 161 | GCA_000966045.1 | 550 | 550 | Enterobacter cloacae | CIDEIMsCOL6 | Proteobacteria | JZKQ01000011.1 | II | 4 |
| 162 | GCA_000966055.1 | 550 | 550 | Enterobacter cloacae | CIDEIMsCOL7 | Proteobacteria | JZKR01000007.1 | II | 4 |
| 163 | GCA_000966085.1 | 550 | 550 | Enterobacter cloacae | CIDEIMsCOL8 | Proteobacteria | JZKS01000009.1 | II | 4 |
| 164 | GCA_000966125.1 | 550 | 550 | Enterobacter cloacae | CIDEIMsCOL11 | Proteobacteria | JZKV01000018.1 | II | 4 |
| 165 | GCA_000968675.1 | 550 | 550 | Enterobacter cloacae | CIDEIMsCOL15 | Proteobacteria | JZKZ01000021.1 | II | 4 |
| 166 | GCA_000235765.3 | 1104326 | 550 | Enterobacter cloacae subsp. dissolvens SDM | SDM | Proteobacteria | CP003678.1 | II | 4 |
| 167 | GCA_001472385.1 | 299766 | 158836 | Enterobacter hormaechei subsp. steigerwaltii | SMART_539 | Proteobacteria | LPPV01000001.1 | II | 4 |
| 168 | GCA_001473115.1 | 299766 | 158836 | Enterobacter hormaechei subsp. steigerwaltii | SMART_1078 | Proteobacteria | LPOL01000074.1 | II | 4 |
| 169 | GCA_000568075.1 | 314330 | 314330 | Enterobacter sp. DC1 | DC1 | Proteobacteria | AZUA01000002.1 | II | 4 |
| 170 | GCA_000010305.1 | 379066 | 173480 | Gemmatimonas aurantiaca T-27 | T-27 | Gemmatimonadetes | AP009153.1 | II | 4 |
| 171 | GCA_000298195.1 | 1108045 | 83341 | Gordonia rhizosphera NBRC 16068 | NBRC 16068 | Actinobacteria | BAHC01000188.1 | II | 3 |
| 172 | GCA_001275395.1 | 1704290 | 1704290 | Hapalosiphon sp. MRB220 | MRB220 | Cyanobacteria | LIRN01000101.1 | II | 3 |
| 173 | GCA_000567145.1 | 1400164 | 573 | Klebsiella pneumoniae BIDMC 46b | BIDMC 46b | Proteobacteria | KK036716.1 | II | 4 |
| 174 | GCA_000529775.1 | 1379689 | 573 | Klebsiella pneumoniae subsp. pneumoniae T69 | T69 | Proteobacteria | HG518478.1 | II | 4 |
| 175 | GCA_000169115.1 | 247639 | 247639 | marine gamma proteobacterium HTCC2080 | HTCC2080 | Proteobacteria | AAVV01000004.1 | II | 3 |
| 176 | GCA_001424165.1 | 1736272 | 1736272 | Massilia sp. Leaf139 | Leaf139 | Proteobacteria | LMOJ01000003.1 | II | 4 |
| 177 | GCA_000970225.1 | 1434114 | 2209 | Methanosarcina mazei LYC | LYC | Euryarchaeota | CP009513.1 | II | 3 |
| 178 | GCA_000757795.1 | 693986 | 334852 | Methylobacterium oryzae CBMB2C | CBMB20 | Proteobacteria | CP003811.1 | II | 4 |
| 179 | GCA_001542815.1 | 925818 | 925818 | Methylobacterium sp. AMS5 | AMS5 | Proteobacteria | CP006992.1 | II | 4 |
| 180 | GCA_000834175.1 | 1587523 | 1587523 | Microbacterium sp. MEJ108Y | MEJ108Y | Actinobacteria | JXQX01000022.1 | II | 3 |
| 181 | GCA_000568915.1 | 457406 | 457406 | Micromonospora sp. M42 | M42 | Actinobacteria | KK037233.1 | II | 3 |
| 182 | GCA_000306785.1 | 477641 | 477641 | Modestobacter marinus | BC501 | Actinobacteria | FO203431.1 | II | 3 |
| 183 | GCA_000307755.2 | 1239989 | 582 | Morganella morganii SC01 | SC01 | Proteobacteria | AMWL02000021.1 | II | 4 |
| 184 | GCA_001570445.1 | 228230 | 228230 | Mycobacterium canariasense | JCM15298 | Actinobacteria | BCSY01000069.1 | II | 3 |
| 185 | GCA_001307545.1 | 1766 | 1766 | Mycobacterium fortuitum | CT6 | Actinobacteria | CP011269.1 | II | 3 |
| 186 | GCA_001187505.1 | 134601 | 134601 | Mycobacterium goodii | X7B | Actinobacteria | CP012150.1 | II | 3 |
| 187 | GCA_001570485.1 | 59813 | 59813 | Mycobacterium novocastrense | JCM18114 | Actinobacteria | BCTA01000023.1 | II | 3 |
| 188 | GCA_000024365.1 | 479431 | 53461 | Nakamurella multipartita DSM 44233 | DSM 44233 | Actinobacteria | CP001737.1 | II | 3 |
| 189 | GCA_000963985.1 | 1628190 | 1628190 | Pantoea sp. BL1 | BL1 | Proteobacteria | JZRH01000052.1 | II | 4 |
| 190 | GCA_001447385.1 | 1745182 | 1745182 | Paracoccussp. MKU1 | MKU1 | Proteobacteria | LLWQ01000019.1 | II | 3 |
| 191 | GCA_000808515.1 | 555 | 554 | Pectobacterium carotovorum subsp. carotovorum | BC T5 | Proteobacteria | JUJS01000001.1 | II | 4 |
| 192 | GCA_000294535.1 | 1218933 | 554 | Pectobacterium carotovorum subsp. carotovorum PCC21 | PCC21 | Proteobacteria | CP003776.1 | II | 4 |
| 193 | GCA_001026985.1 | 82985 | 82985 | Pragia fontium | 24613 | Proteobacteria | CP010423.1 | II | 4 |
| 194 | GCA_000829255.1 | 287 | 287 | Pseudomonas aeruginosa | NCGM 1984 | Proteobacteria | AP014646.1 | II | 3 |
| 195 | GCA_000829275.1 | 287 | 287 | Pseudomonas aeruginosa | NCGM 1900 | Proteobacteria | AP014622.1 | II | 3 |
| 196 | GCA_001449645.1 | 287 | 287 | Pseudomonas aeruginosa | WH-SGI-V-07177 | Proteobacteria | LLMG01000101.1 | II | 3 |
| 197 | GCA_001449765.1 | 287 | 287 | Pseudomonas aeruginosa | WH-SGI-V-07186 | Proteobacteria | LLMQ01000093.1 | II | 3 |
| 198 | GCA_001450265.1 | 287 | 287 | Pseudomonas aeruginosa | WH-SGI-V-07378 | Proteobacteria | LLOC01000126.1 | II | 4 |
| 199 | GCA_001450505.1 | 287 | 287 | Pseudomonas aeruginosa | WH-SGI-V-07170 | Proteobacteria | LLLZ01000038.1 | II | 4 |
| 200 | GCA_001450545.1 | 287 | 287 | Pseudomonas aeruginosa | WH-SGI-V-07176 | Proteobacteria | LLMF01000085.1 | II | 3 |
| 201 | GCA_001450995.1 | 287 | 287 | Pseudomonas aeruginosa | WH-SGI-V-07387 | Proteobacteria | LLOL01000018.1 | II | 4 |
| 202 | GCA_001451125.1 | 287 | 287 | Pseudomonas aeruginosa | WH-SGI-V-07406 | Proteobacteria | LLOT01000015.1 | II | 4 |
| 203 | GCA_001451485.1 | 287 | 287 | Pseudomonas aeruginosa | WH-SGI-V-07425 | Proteobacteria | LLPM01000023.1 | II | 4 |
| 204 | GCA_001451605.1 | 287 | 287 | Pseudomonas aeruginosa | WH-SGI-V-07486 | Proteobacteria | LLPSO1000097.1 | II | 4 |
| 205 | GCA_001451625.1 | 287 | 287 | Pseudomonas aeruginosa | WH-SGI-V-07487 | Proteobacteria | LLPTO1000056.1 | II | 4 |
| 206 | GCA_001451815.1 | 287 | 287 | Pseudomonas aeruginosa | WH-SGI-V-07498 | Proteobacteria | LLQE01000017.1 | II | 4 |
| 207 | GCA_001451885.1 | 287 | 287 | Pseudomonas aeruginosa | WH-SGI-V-07504 | Proteobacteria | LLQK01000005.1 | II | 4 |
| 208 | GCA_001451985.1 | 287 | 287 | Pseudomonas aeruginosa | WH-SGI-V-07622 | Proteobacteria | LLQT01000086.1 | II | 4 |
| 209 | GCA_001452025.1 | 287 | 287 | Pseudomonas aeruginosa | WH-SGI-V-07626 | Proteobacteria | LLQX01000033.1 | II | 4 |
| 210 | GCA_001452045.1 | 287 | 287 | Pseudomonas aeruginosa | WH-SGI-V-07627 | Proteobacteria | LLQY01000018.1 | II | 4 |
| 211 | GCA_001452305.1 | 287 | 287 | Pseudomonas aeruginosa | WH-SGI-V-07645 | Proteobacteria | LLRQ01000036.1 | II | 4 |
| 212 | GCA_001452325.1 | 287 | 287 | Pseudomonas aeruginosa | WH-SGI-V-07647 | Proteobacteria | LLRS01000111.1 | II | 3 |
| 213 | GCA_001452575.1 | 287 | 287 | Pseudomonas aeruginosa | WH-SGI-V-07694 | Proteobacteria | LLSO01000025.1 | II | 4 |
| 214 | GCA_001452675.1 | 287 | 287 | Pseudomonas aeruginosa | WH-SGI-V-07703 | Proteobacteria | LLSX01000034.1 | II | 4 |
| 215 | GCA_001453345.1 | 287 | 287 | Pseudomonas aeruginosa | WH-SGI-V-07624 | Proteobacteria | LLQV01000015.1 | II | 4 |
| 216 | GCA_001453365.1 | 287 | 287 | Pseudomonas aeruginosa | WH-SGI-V-07625 | Proteobacteria | LLQW01000076.1 | II | 4 |
| 217 | GCA_001453415.1 | 287 | 287 | Pseudomonas aeruginosa | WH-SGI-V-07632 | Proteobacteria | LLRD01000075.1 | II | 3 |
| 218 | GCA_001453485.1 | 287 | 287 | Pseudomonas aeruginosa | WH-SGI-V-07646 | Proteobacteria | LLRR01000027.1 | II | 4 |
| 219 | GCA_001453565.1 | 287 | 287 | Pseudomonas aeruginosa | WH-SGI-V-07679 | Proteobacteria | LLRZ01000123.1 | II | 4 |
| 220 | GCA_001453595.1 | 287 | 287 | Pseudomonas aeruginosa | WH-SGI-V-07683 | Proteobacteria | LLSD01000091.1 | II | 4 |
| 221 | GCA_001454045.1 | 287 | 287 | Pseudomonas aeruginosa | WH-SGI-V-07276 | Proteobacteria | LLUCO1000021.1 | II | 4 |
| 222 | GCA_001454305.1 | 287 | 287 | Pseudomonas aeruginosa | WH-SGI-V-07309 | Proteobacteria | LLUY01000055.1 | II | 4 |
| 223 | GCA_001454595.1 | 287 | 287 | Pseudomonas aeruginosa | WH-SGI-V-07268 | Proteobacteria | LLTZ01000033.1 | II | 4 |
| 224 | GCA_000148745.1 | 798130 | 287 | Pseudomonas aeruginosa 39016 | 39016 | Proteobacteria | CM001020.1 | II | 3 |
| 225 | GCA_000481745.1 | 1402498 | 287 | Pseudomonas aeruginosa 6077 | 6077 | Proteobacteria | KI519216.1 | II | 4 |
| 226 | GCA_000480985.1 | 1402549 | 287 | Pseudomonas aeruginosa BL08 | BL08 | Proteobacteria | KI518899.1 | II | 4 |
| 227 | GCA_000480865.1 | 1402555 | 287 | Pseudomonas aeruginosa BL14 | BL14 | Proteobacteria | KI518844.1 | II | 4 |
| 228 | GCA_000480805.1 | 1402558 | 287 | Pseudomonas aeruginosa BL17 | BL17 | Proteobacteria | KI518822.1 | II | 4 |
| 229 | GCA_000629125.1 | 1447538 | 287 | Pseudomonas aeruginosa BWH030 | BWH030 | Proteobacteria | KK212308.1 | II | 4 |
| 230 | GCA_000629585.1 | 1418236 | 287 | Pseudomonas aeruginosa BWH050 | BWH050 | Proteobacteria | KK213277.1 | II | 4 |
| 231 | GCA_000481585.1 | 1402506 | 287 | Pseudomonas aeruginosa BWHPSA006 | BWHPSA006 | Proteobacteria | KI519145.1 | II | 4 |
| 232 | GCA_000480435.1 | 1402576 | 287 | Pseudomonas aeruginosa C48 | C48 | Proteobacteria | KI518703.1 | II | 4 |
| 233 | GCA_000481805.1 | 1402495 | 287 | Pseudomonas aeruginosa JJ692 | JJ692 | Proteobacteria | KI519252.1 | II | 4 |
| 234 | GCA_000513235.1 | 1306165 | 287 | Pseudomonas aeruginosa MH27 | MH27 | Proteobacteria | CBTR010000015.1 | II | 3 |
| 235 | GCA_000284555.1 | 1089456 | 287 | Pseudomonas aeruginosa NCGM2.S1 | NCGM2.S1 | Proteobacteria | AP012280.1 | II | 3 |
| 236 | GCA_000481785.1 | 1402496 | 287 | Pseudomonas aeruginosa U2504 | U2504 | Proteobacteria | KI519240.1 | II | 4 |
| 237 | GCA_000786485.1 | 1341645 | 287 | Pseudomonas aeruginosa WS394 | WS394 | Proteobacteria | CBYA010000116.1 | II | 4 |
| 238 | GCA_000012265.1 | 220664 | 380021 | Pseudomonas protegens Pf-5 | Pf-5 | Proteobacteria | CP000076.1 | II | 4 |
| 239 | GCA_000334035.1 | 1204471 | 317 | Pseudomonas syringae BRIP34876 | BRIP34876 | Proteobacteria | KB316468.1 | II | 4 |
| 240 | GCA_000334055.1 | 1205751 | 317 | Pseudomonas syringae BRIP34881 | BRIP34881 | Proteobacteria | KB316535.1 | II | 4 |
| 241 | GCA_000765305.1 | 323 | 251701 | Pseudomonas syringae pv. tomato | NYS-T1 | Proteobacteria | KN265560.1 | II | 4 |
| 242 | GCA_001518465.1 | 323 | 251701 | Pseudomonas syringae pv. tomato | A9 | Proteobacteria | LNKY01000101.1 | II | 4 |
| 243 | GCA_001518695.1 | 323 | 251701 | Pseudomonas syringae pv. tomato | 407 | Proteobacteria | LNKZ01000069.1 | II | 4 |
| 244 | GCA_000172895.1 | 546231 | 251701 | Pseudomonas syringae pv. tomato T1 | T1 | Proteobacteria | ABSM01000016.1 | II | 4 |
| 245 | GCA_000471625.1 | 1354303 | 248452 | Psychrobacter aquaticus CMS 56 | CMS 56 | Proteobacteria | AUSW01000035.1 | II | 3 |
| 246 | GCA_001297245.1 | 1538158 | 1538158 | Rhizobium acidisoli | FH23 | Proteobacteria | LJSR01000033.1 | II | 4 |
| 247 | GCA_001425345.1 | 1736357 | 1736357 | Rhizobium sp. Leaf383 | Leaf383 | Proteobacteria | LMQD01000026.1 | II | 3 |
| 248 | GCA_001507595.1 | 1685379 | 1685379 | Ruegeria sp. ZGT118 | ZGT118 | Proteobacteria | LQBQ01000001.1 | II | 3 |
| 249 | GCA_001474275.1 | 28901 | 28901 | Salmonella enterica | CVM 43767 | Proteobacteria | JYQK01000006.1 | II | 4 |
| 250 | GCA_001569645.1 | 28901 | 28901 | Salmonella enterica | NGUA06 | Proteobacteria | BCOE01000051.1 | II | 4 |
| 251 | GCA_001569685.1 | 28901 | 28901 | Salmonella enterica | NGUA08 | Proteobacteria | BCOG01000098.1 | II | 4 |
| 252 | GCA_001569765.1 | 28901 | 28901 | Salmonella enterica | NGUA12 | Proteobacteria | BCOK01000075.1 | II | 4 |
| 253 | GCA_001569785.1 | 28901 | 28901 | Salmonella enterica | NGUA13 | Proteobacteria | BCOL01000051.1 | II | 4 |
| 254 | GCA_001569865.1 | 28901 | 28901 | Salmonella enterica | NGUA17 | Proteobacteria | BCOP01000026.1 | II | 4 |
| 255 | GCA_001569905.1 | 28901 | 28901 | Salmonella enterica | NGUA19 | Proteobacteria | BCOR01000051.1 | II | 4 |
| 256 | GCA_001570025.1 | 28901 | 28901 | Salmonella enterica | NGUA25 | Proteobacteria | BCOX01000056.1 | II | 4 |
| 257 | GCA_001570065.1 | 28901 | 28901 | Salmonella enterica | NGUA27 | Proteobacteria | BCOZ01000088.1 | II | 4 |
| 258 | GCA_001570105.1 | 28901 | 28901 | Salmonella enterica | NGUA29 | Proteobacteria | BCPB01000051.1 | II | 4 |
| 259 | GCA_001570145.1 | 28901 | 28901 | Salmonella enterica | NGUA31 | Proteobacteria | BCPD01000090.1 | II | 4 |
| 260 | GCA_001570205.1 | 28901 | 28901 | Salmonella enterica | NGUA34 | Proteobacteria | BCPG01000042.1 | II | 4 |
| 261 | GCA_001448925.1 | 1245396 | 28901 | Salmonella enterica subsp. arizonae serovar 62:z36:- str. 5335/86 | 5335/86 | Proteobacteria | LHTK01000001.1 | II | 4 |
| 262 | GCA_000756465.1 | 1386967 | 28901 | Salmonella enterica subsp. arizonae serovar 62:z36:- str. | RKS2983 | Proteobacteria | CP006693.1 | II | 4 |
| | | | | RKS2983 | | | | | |
| 263 | GCA_000748945.1 | 1173471 | 28901 | Salmonella enterica subsp.enterica serovar Bareilly str. | CFSAN000235 | Proteobacteria | JRCT01000004.1 | II | 4 |
| | | | | CFSAN000235 | | | | | |
| 264 | GCA_000748965.1 | 1173472 | 28901 | Salmonella enterica subsp. enterica serovar Bareilly str. | CFSAN000236 | Proteobacteria | JRCS01000009.1 | II | 3 |
| | | | | CFSAN000236 | | | | | |
| 265 | GCA_000749105.1 | 1182179 | 28901 | Salmonella enterica subsp. enterica serovar Bareilly str. | CFSAN000754 | Proteobacteria | JRCL01000011.1 | II | 4 |
| | | | | CFSAN000754 | | | | | |
| 266 | GCA_000504705.2 | 1192560 | 28901 | Salmonella enterica subsp. enterica serovar Cubana str. 76814 | 76814 | Proteobacteria | KI635640.1 | II | 4 |
| 267 | GCA_000430125.1 | 1271863 | 28901 | Salmonella enterica subsp. enterica serovar Cubana str. | CFSAN002050 | Proteobacteria | CP006055.1 | II | 4 |
| | | | | CFSAN002050 | | | | | |
| 268 | GCA_000500025.1 | 1314885 | 28901 | Salmonella enterica subsp. enterica serovar Cubana str. | CVM42234 | Proteobacteria | ATEU01000012.1 | II | 4 |
| | | | | CVM42234 | | | | | |
| 269 | GCA_000487835.1 | 1194151 | 28901 | Salmonella enterica subsp. enterica serovar Norwich str. | CFSAN001077 | Proteobacteria | APAM01000054.1 | II | 4 |
| | | | | CFSAN001077 | | | | | |
| 270 | GCA_000486285.1 | 984254 | 28901 | Salmonella enterica subsp. enterica serovar Virchow str. ATCC | ATCC 51955 | Proteobacteria | AOXL01000118.1 | II | 4 |
| | | | | 51955 | | | | | |
| 271 | GCA_000171535.2 | 465517 | 28901 | Salmonella enterica subsp. enterica serovar Virchow str. SL491 | SL491 | Proteobacteria | ABFH02000001.1 | II | 3 |
| 272 | GCA_000516855.1 | 1249525 | 28901 | Salmonella enterica subsp. enterica serovar Virchow str. SVQ1 | SVQ1 | Proteobacteria | KI911915.1 | II | 4 |
| 273 | GCA_001409135.1 | 57743 | 28901 | Salmonella enterica subsp. enterica serovar Weltevreden | 2511STDY5462388 | Proteobacteria | LN890518.1 | II | 4 |
| 274 | GCA_001409155.1 | 57743 | 28901 | Salmonella enterica subsp. enterica serovar Weltevreden | 2511STDY5712384 | Proteobacteria | LN890522.1 | II | 4 |
| 275 | GCA_001409175.1 | 57743 | 28901 | Salmonella enterica subsp. enterica serovar Weltevreden | 2511STDY5712385 | Proteobacteria | LN890524.1 | II | 4 |
| 276 | GCA_001409195.1 | 57743 | 28901 | Salmonella enterica subsp. enterica serovar Weltevreden | 2511STDY5462413 | Proteobacteria | LN890520.1 | II | 4 |
| 277 | GCA_000230505.1 | 936157 | 28901 | Salmonella enterica subsp. enterica serovar Weltevreden str. | 2007-60-3289-1 | Proteobacteria | FR775246.1 | II | 4 |
| | | | | 2007-60-3289-1 | | | | | |
| 278 | GCA_000171495.1 | 465518 | 28901 | Salmonella enterica subsp. enterica serovar Weltevreden str. | HI_N05-537 | Proteobacteria | ABFF01000011.1 | II | 4 |
| | | | | HI_N05-537 | | | | | |
| 279 | GCA_001188155.1 | 59205 | 28901 | Salmonella enterica subsp. houtenae | 01-0133 | Proteobacteria | JWSP01000004.1 | II | 4 |
| 280 | GCA_000486245.1 | 1173948 | 28901 | Salmonella enterica subsp. houtenae serovar 50:g, z51:- str. 01- | 01-0133 | Proteobacteria | AOXJ01000080.1 | II | 3 |
| | | | | 0133 | | | | | |
| 281 | GCA_000521925.1 | 1400186 | 615 | Serratia marcescens BIDMC 44 | BIDMC 44 | Proteobacteria | KI929263.1 | II | 4 |
| 282 | GCA_000633695.1 | 1445865 | 615 | Serratia marcescens BIDMC 81 | BIDMC 81 | Proteobacteria | KK214290.1 | II | 4 |
| 283 | GCA_000006965.1 | 266834 | 382 | Sinorhizobium meliloti 1021 | 1021 | Proteobacteria | AL591688.1 | II | 3 |
| 284 | GCA_000346065.1 | 1286640 | 382 | Sinorhizobium meliloti 2011 | 2011 | Proteobacteria | CP004140.1 | II | 3 |
| 285 | GCA_001461685.1 | 1358418 | 1358418 | Sinorhizobium sp. GL28 | GL28 | Proteobacteria | AUSZ01000302.1 | II | 4 |
| 286 | GCA_000280865.1 | 1160718 | 114687 | Streptomyces auratus AGR0001 | AGR0001 | Actinobacteria | JH725387.1 | II | 3 |
| 287 | GCA_001188955.1 | 1894 | 1894 | Streptomyces aureofaciens | ATCC 10762 | Actinobacteria | KQ257755.1 | II | 3 |
| 288 | GCA_000222625.2 | 1051645 | 29497 | Vibrio splendidus ATCC 33789 | ATCC 33789 | Proteobacteria | AFWG01000006.1 | II | 3 |
| 289 | GCA_001139945.1 | 29483 | 29483 | Yersinia aldovae | IP08791 | Proteobacteria | CQEH01000010.1 | II | 4 |
| 290 | GCA_001104645.1 | 631 | 631 | Yersinia intermedia | FCF335 | Proteobacteria | CQCT01000008.1 | II | 4 |
| 291 | GCA_001144105.1 | 633 | 633 | Yersinia pseudotuberculosis | N912 | Proteobacteria | CQDI01000025.1 | II | 4 |
| 292 | GCA_001166025.1 | 633 | 633 | Yersinia pseudotuberculosis | OK5586 | Proteobacteria | CQEB01000011.1 | II | 4 |
| 293 | GCA_001319685.1 | 633 | 633 | Yersinia pseudotuberculosis | IP32921 | Proteobacteria | CIFM01000002.1 | II | 4 |
| 294 | GCA_000834455.1 | 29485 | 29485 | Yersinia rohdei | YRA | Proteobacteria | CP009787.1 | II | 4 |
| 295 | GCA_000173775.1 | 527004 | 29485 | Yersinia rohdei ATCC 43380 | ATCC 43380 | Proteobacteria | ACCD01000010.1 | II | 4 |
| 296 | GCA_001581075.1 | 178900 | 178900 | Acetobacter cerevisiae | LMG 1608 | Proteobacteria | LHZY01000057.1 | III | 2 |
| 297 | GCA_001077655.1 | 470 | 470 | Acinetobacter baumannii | Ab04-mff | Proteobacteria | CP012006.1 | III | 2 |
| 298 | GCA_001276085.1 | 470 | 470 | Acinetobacter baumannii | 5021_13 | Proteobacteria | LGYV01000089.1 | III | 2 |
| 299 | GCA_001441545.1 | 470 | 470 | Acinetobacter baumannii | MRSN15038 | Proteobacteria | LMZX01000006.1 | III | 2 |
| 300 | GCA_001441555.1 | 470 | 470 | Acinetobacter baumannii | MRSN20315 | Proteobacteria | LMZV01000012.1 | III | 2 |
| 301 | GCA_001441565.1 | 470 | 470 | Acinetobacter baumannii | MRSN15084 | Proteobacteria | LMZW01000004.1 | III | 2 |
| 302 | GCA_001441625.1 | 470 | 470 | Acinetobacter baumannii | MRSN20755 | Proteobacteria | LMYO01000010.1 | III | 2 |
| 303 | GCA_001441645.1 | 470 | 470 | Acinetobacter baumannii | MRSN7081 | Proteobacteria | LMZY01000005.1 | III | 2 |
| 304 | GCA_000581835.1 | 1310681 | 470 | Acinetobacter baumannii 1542444 | 1542444 | Proteobacteria | JEYA01000001.1 | III | 2 |
| 305 | GCA_000581295.1 | 1310630 | 470 | Acinetobacter baumannii 99063 | 99063 | Proteobacteria | JEXJ01000065.1 | III | 2 |
| 306 | GCA_000335595.1 | 903937 | 470 | Acinetobacter baumannii AA-014 | AA-014 | Proteobacteria | AMGA01000013.1 | III | 2 |
| 307 | GCA_000369165.1 | 1217630 | 470 | Acinetobacter baumannii NIPH 201 | NIPH 201 | Proteobacteria | KB849843.1 | III | 2 |
| 308 | GCA_000369205.1 | 1217633 | 470 | Acinetobacter baumannii NIPH 335 | NIPH 335 | Proteobacteria | KB849885.1 | III | 2 |
| 309 | GCA_000369265.1 | 1217627 | 470 | Acinetobacter baumannii NIPH 67 | NIPH 67 | Proteobacteria | KB849903.1 | III | 2 |
| 310 | GCA_000309235.1 | 903926 | 470 | Acinetobacter baumannii OIFC035 | OIFC035 | Proteobacteria | AMTB01000008.1 | III | 2 |
| 311 | GCA_000305235.1 | 903902 | 470 | Acinetobacter baumannii OIFC098 | OIFC098 | Proteobacteria | AMDF01000045.1 | III | 2 |
| 312 | GCA_000368485.1 | 1217656 | 106649 | Acinetobacter guillouiae NIPH 991 | NIPH 991 | Proteobacteria | KB849456.1 | III | 2 |
| 313 | GCA_000369685.1 | 1144665 | 1144665 | Acinetobacter sp. CIP 102136 | CIP 102136 | Proteobacteria | KB850142.1 | III | 2 |
| 314 | GCA_000368425.1 | 1144669 | 1144669 | Acinetobacter sp. CIP 102637 | CIP 102637 | Proteobacteria | KB849435.1 | III | 2 |
| 315 | GCA_000154465.1 | 445970 | 28117 | Alistipes putredinis DSM 17216 | DSM 17216 | Bacteroidetes | DS499580.1 | III | 2 |
| 316 | GCA_001578515.1 | 28108 | 28108 | Alteromonas macleodii | HOT1A3 | Proteobacteria | CP012202.1 | III | 2 |
| 317 | GCA_000300175.1 | 1004787 | 28108 | Alteromonas macleodii str. ‘Balearic Sea AD45’ | Balearic Sea AD45 | Proteobacteria | CP003873.1 | III | 2 |
| 318 | GCA_000204015.1 | 715226 | 76891 | Asticcacaulis biprosthecum C19 | C19 | Proteobacteria | GL883078.1 | III | 2 |
| 319 | GCA_000495835.1 | 1282361 | 1282361 | Asticcacaulis sp. AC402 | AC402 | Proteobacteria | AWGC01000023.1 | III | 2 |
| 320 | GCA_000061505.1 | 62928 | 62928 | Azoarcus sp. BH72 | BH72 | Proteobacteria | AM406670.1 | III | 2 |
| 321 | GCA_000817975.1 | 1328314 | 353 | Azotobacter chroococcum NCIMB 8003 | NCIMB 8003 | Proteobacteria | CP010421.1 | III | 2 |
| 322 | GCA_001286525.1 | 817 | 817 | Bacteroides fragilis | BE1 | Bacteroidetes | LN877293.1 | III | 2 |
| 323 | GCA_000210835.1 | 862962 | 817 | Bacteroides fragilis 638R | 638R | Bacteroidetes | FQ312004.1 | III | 2 |
| 324 | GCA_000598705.1 | 1339297 | 817 | Bacteroides fragilis str. 1009-4-F #10 | 1009-4-F #10 | Bacteroidetes | JGED01000021.1 | III | 2 |
| 325 | GCA_000599285.2 | 1339296 | 817 | Bacteroides fragilis str. 1009-4-F #7 | 1009-4-F #7 | Bacteroidetes | JGEE01000031.1 | III | 2 |
| 326 | GCA_000598325.1 | 1339309 | 817 | Bacteroides fragilis str. 3783N1-2 | 3783N1-2 | Bacteroidetes | JGCS01000280.1 | III | 2 |
| 327 | GCA_000599065.2 | 1339310 | 817 | Bacteroides fragilis str. 3783N1-6 | 3783N1-6 | Bacteroidetes | JGEU01000034.1 | III | 2 |
| 328 | GCA_000598605.1 | 1339311 | 817 | Bacteroides fragilis str. 3783N1-8 | 3783N1-8 | Bacteroidetes | JGDR01000052.1 | III | 2 |
| 329 | GCA_000598345.1 | 1339312 | 817 | Bacteroides fragilis str. 3783N2-1 | 3783N2-1 | Bacteroidetes | JGCT01000263.1 | III | 2 |
| 330 | GCA_000598165.1 | 1339313 | 817 | Bacteroides fragilis str. 3976T7 | 3976T7 | Bacteroidetes | JGCU01000310.1 | III | 2 |
| 331 | GCA_000154525.1 | 449673 | 46506 | Bacteroides stercoris ATCC 43183 | ATCC 43183 | Bacteroidetes | DS499662.1 | III | 2 |
| 332 | GCA_001578555.1 | 820 | 820 | Bacteroides uniformis | KLE1607 | Bacteroidetes | KQ968311.1 | III | 2 |
| 333 | GCA_000224655.1 | 693988 | 693988 | Bilophila sp. 4_1_30 | 4_1_30 | Proteobacteria | JH114233.1 | III | 2 |
| 334 | GCA_000173575.1 | 516466 | 516466 | Burkholderia sp. H160 | H160 | Proteobacteria | ABYL01000008.1 | III | 2 |
| 335 | GCA_001527325.1 | 101571 | 101571 | Burkholderia ubonensis | MSMB2054 | Proteobacteria | LOZC01000078.1 | III | 2 |
| 336 | GCA_001527345.1 | 101571 | 101571 | Burkholderia ubonensis | MSMB2055 | Proteobacteria | LOZD01000059.1 | III | 2 |
| 337 | GCA_001527425.1 | 101571 | 101571 | Burkholderia ubonensis | MSMB2061 | Proteobacteria | LOZH01000090.1 | III | 2 |
| 338 | GCA_001529135.1 | 101571 | 101571 | Burkholderia ubonensis | MSMB1212WGS | Proteobacteria | LPED01000119.1 | III | 2 |
| 339 | GCA_001531435.1 | 101571 | 101571 | Burkholderia ubonensis | MSMB1508WGS | Proteobacteria | LPFT01000065.1 | III | 2 |
| 340 | GCA_000531125.1 | 1400861 | 1400860 | Candidatus Contendobacter odensis Run_B_J11 | Run_B_J11 | Proteobacteria | CBTK010000022.1 | III | 2 |
| 341 | GCA_001292585.1 | 1509431 | 1509431 | Candidatus Magnetomorum sp. HK-1 | | Proteobacteria | JPDT01000292.1 | III | 2 |
| 342 | GCA_000477035.2 | 1348585 | 76594 | Cellulophaga baltica NN016038 | NN016038 | Bacteroidetes | CP009887.1 | III | 2 |
| 343 | GCA_000601485.1 | 1192034 | 51 | Chondromyces apiculatus DSM 436 | DSM 436 | Proteobacteria | ASRX01000002.1 | III | 2 |
| 344 | GCA_001421435.1 | 1735672 | 1735672 | Chryseobacterium sp. Leaf201 | Leaf201 | Bacteroidetes | LMKA01000095.1 | III | 2 |
| 345 | GCA_001281005.1 | 546 | 546 | Citrobacter freundii | P10159 | Proteobacteria | CP012554.1 | III | 2 |
| 346 | GCA_001316675.1 | 546 | 546 | Citrobacter freundii | ST62:944112508 | Proteobacteria | LJEB01000070.1 | III | 2 |
| 347 | GCA_001034465.1 | 1686377 | 1686377 | Citrobacter sp. MGH100 | MGH100 | Proteobacteria | KQ089800.1 | III | 2 |
| 348 | GCA_001037515.1 | 1686383 | 1686383 | Citrobacter sp. MGH110 | MGH110 | Proteobacteria | KQ089830.1 | III | 2 |
| 349 | GCA_000828895.1 | 1458425 | 1458425 | Comamonadaceae bacterium A1 | A1 | Proteobacteria | AP014568.1 | III | 2 |
| 350 | GCA_001277275.1 | 28141 | 28141 | Cronobacter sakazakii | NCTC 8155 | Proteobacteria | CP012253.1 | III | 2 |
| 351 | GCA_000339015.1 | 956149 | 28141 | Cronobacter sakazakii SP291 | Sp291 | Proteobacteria | CP004091.1 | III | 2 |
| 352 | GCA_000961725.1 | 1629713 | 1629713 | Desulfobulbaceae bacterium BRH_c16a | | Proteobacteria | LADS01000053.1 | III | 2 |
| 353 | GCA_001011305.1 | 548 | 548 | Enterobacter aerogenes | GN02420 | Proteobacteria | LDBI01000017.1 | III | 2 |
| 354 | GCA_001011595.1 | 548 | 548 | Enterobacter aerogenes | GN03959 | Proteobacteria | LDBV01000046.1 | III | 2 |
| 355 | GCA_001030055.1 | 548 | 548 | Enterobacter aerogenes | UCI98 | Proteobacteria | LESY01000010.1 | III | 2 |
| 356 | GCA_001030185.1 | 548 | 548 | Enterobacter aerogenes | UCI97 | Proteobacteria | KQ087587.1 | III | 2 |
| 357 | GCA_001022755.1 | 61645 | 61645 | Enterobacter asburiae | GN02692 | Proteobacteria | LEDI01000011.1 | III | 2 |
| 358 | GCA_000952375.1 | 550 | 550 | Enterobacter cloacae | MNCRE12 | Proteobacteria | JYME01000006.1 | III | 2 |
| 359 | GCA_001022875.1 | 550 | 550 | Enterobacter cloacae | GN02773 | Proteobacteria | LEDE01000006.1 | III | 2 |
| 360 | GCA_001030145.1 | 550 | 550 | Enterobacter cloacae | UCI86 | Proteobacteria | KQ087579.1 | III | 2 |
| 361 | GCA_001030195.1 | 550 | 550 | Enterobacter cloacae | UCI85 | Proteobacteria | KQ087593.1 | III | 2 |
| 362 | GCA_001518245.1 | 550 | 550 | Enterobacter cloacae | GN03848 | Proteobacteria | LRCM01000015.1 | III | 2 |
| 363 | GCA_001518625.1 | 550 | 550 | Enterobacter cloacae | GN04311 | Proteobacteria | LRCQ01000112.1 | III | 2 |
| 364 | GCA_001317055.1 | 336306 | 550 | Enterobacter cloacae subsp. cloacae | ST433:953102574 | Proteobacteria | LJE001000030.1 | III | 2 |
| 365 | GCA_000492435.1 | 1329856 | 550 | Enterobacter cloacae UCICRE 12 | UCICRE 12 | Proteobacteria | KI535461.1 | III | 2 |
| 366 | GCA_001037885.1 | 1686400 | 1686400 | Enterobacter sp. BIDMC109 | BIDMC109 | Proteobacteria | KQ090002.1 | III | 2 |
| 367 | GCA_000735105.1 | 421072 | 421072 | Epilithonimonas lactis | LMG 24401 | Bacteroidetes | JPLY01000004.1 | III | 2 |
| 368 | GCA_001077815.1 | 1648404 | 1648404 | Erythrobacter sp. s21-N3 | s21-N3 | Proteobacteria | CP011310.1 | III | 2 |
| 369 | GCA_000181515.1 | 161528 | 161528 | Erythrobacter sp. SD-21 | SD-21 | Proteobacteria | ABCG01000002.1 | III | 2 |
| 370 | GCA_000695175.1 | 562 | 562 | Escherichia coli | MI10-01 | Proteobacteria | JHEZ01000002.1 | III | 2 |
| 371 | GCA_000731455.1 | 562 | 562 | Escherichia coli | D6-117_07.11 | Proteobacteria | HG976958.1 | III | 2 |
| 372 | GCA_000797755.1 | 562 | 562 | Escherichia coli | CVM N33443PS | Proteobacteria | JUDP01000006.1 | III | 2 |
| 373 | GCA_000797775.1 | 562 | 562 | Escherichia coli | CVM N34322PS | Proteobacteria | JUCY01000038.1 | III | 2 |
| 374 | GCA_000797995.1 | 562 | 562 | Escherichia coli | CVM N36613PS | Proteobacteria | JUCH01000028.1 | III | 2 |
| 375 | GCA_000798215.1 | 562 | 562 | Escherichia coli | CVM N36620PS | Proteobacteria | JUCG01000047.1 | III | 2 |
| 376 | GCA_000798415.1 | 562 | 562 | Escherichia coli | CVM N34557PS | Proteobacteria | JUCU01000037.1 | III | 2 |
| 377 | GCA_000798575.1 | 562 | 562 | Escherichia coli | CVM N36404PS | Proteobacteria | JUCJ01000011.1 | III | 2 |
| 378 | GCA_000819265.1 | 562 | 562 | Escherichia coli | CVM N38428PS | Proteobacteria | JWYX01000074.1 | III | 2 |
| 379 | GCA_000819645.1 | 562 | 562 | Escherichia coli | | Proteobacteria | CP010315.1 | III | 2 |
| 380 | GCA_000934865.1 | 562 | 562 | Escherichia coli | ATCC 35150 | Proteobacteria | JXUS01000117.1 | III | 2 |
| 381 | GCA_000935075.1 | 562 | 562 | Escherichia coli | ATCC 35150 NalR | Proteobacteria | JYIO01000082.1 | III | 2 |
| 382 | GCA_000953765.1 | 562 | 562 | Escherichia coli | MCI_507 | Proteobacteria | JZSD01000085.1 | III | 2 |
| 383 | GCA_000965565.1 | 562 | 562 | Escherichia coli | St. Olav172 | Proteobacteria | JZDY01000017.1 | III | 2 |
| 384 | GCA_000965655.1 | 562 | 562 | Escherichia coli | St. Olav173 | Proteobacteria | JZDZ01000061.1 | III | 2 |
| 385 | GCA_001012075.1 | 562 | 562 | Escherichia coli | CFSAN026805 | Proteobacteria | LDCU01000029.1 | III | 2 |
| 386 | GCA_001030405.1 | 562 | 562 | Escherichia coli | MGH108 | Proteobacteria | KQ087687.1 | III | 2 |
| 387 | GCA_001191125.1 | 562 | 562 | Escherichia coli | CFSAN026804 | Proteobacteria | LGZD01000035.1 | III | 2 |
| 388 | GCA_001191185.1 | 562 | 562 | Escherichia coli | CFSAN026847 | Proteobacteria | LGZE01000020.1 | III | 2 |
| 389 | GCA_001191195.1 | 562 | 562 | Escherichia coli | CFSAN026846 | Proteobacteria | LGZI01000146.1 | III | 2 |
| 390 | GCA_001191525.1 | 562 | 562 | Escherichia coli | CFSAN026848 | Proteobacteria | LHAB01000019.1 | III | 2 |
| 391 | GCA_001263735.1 | 562 | 562 | Escherichia coli | INSLA289 | Proteobacteria | LHAT01000022.1 | III | 2 |
| 392 | GCA_001281685.1 | 562 | 562 | Escherichia coli | STEC 771 | Proteobacteria | LGAZ01000005.1 | III | 2 |
| 393 | GCA_001281725.1 | 562 | 562 | Escherichia coli | STEC 2868 | Proteobacteria | LGBJ01000009.1 | III | 2 |
| 394 | GCA_001281755.1 | 562 | 562 | Escherichia coli | E09/224 | Proteobacteria | LGBP01000024.1 | III | 2 |
| 395 | GCA_001281775.1 | 562 | 562 | Escherichia coli | STEC 989 | Proteobacteria | LGBA01000013.1 | III | 2 |
| 396 | GCA_001281795.1 | 562 | 562 | Escherichia coli | STEC 994 | Proteobacteria | LGBB01000045.1 | III | 2 |
| 397 | GCA_001281815.1 | 562 | 562 | Escherichia coli | STEC 1109 | Proteobacteria | LGBC01000012.1 | III | 2 |
| 398 | GCA_001281845.1 | 562 | 562 | Escherichia coli | STEC 2075 | Proteobacteria | LGBD01000011.1 | III | 2 |
| 399 | GCA_001281855.1 | 562 | 562 | Escherichia coli | STEC 2112 | Proteobacteria | LGBE01000001.1 | III | 2 |
| 400 | GCA_001281885.1 | 562 | 562 | Escherichia coli | STEC 2257 | Proteobacteria | LGBF01000023.1 | III | 2 |
| 401 | GCA_001281905.1 | 562 | 562 | Escherichia coli | STEC 2410 | Proteobacteria | LGBG01000006.1 | III | 2 |
| 402 | GCA_001281915.1 | 562 | 562 | Escherichia coli | STEC 2821 | Proteobacteria | LGBI01000140.1 | III | 2 |
| 403 | GCA_001281925.1 | 562 | 562 | Escherichia coli | STEC 2667 | Proteobacteria | LGBH01000007.1 | III | 2 |
| 404 | GCA_001281965.1 | 562 | 562 | Escherichia coli | E09/10 | Proteobacteria | LGBK01000008.1 | III | 2 |
| 405 | GCA_001281985.1 | 562 | 562 | Escherichia coli | EPEC 287 | Proteobacteria | LGBL01000025.1 | III | 2 |
| 406 | GCA_001281995.1 | 562 | 562 | Escherichia coli | EPEC 393 | Proteobacteria | LGBM01000019.1 | III | 2 |
| 407 | GCA_001282025.1 | 562 | 562 | Escherichia coli | EPEC 1572 | Proteobacteria | LGBN01000001.1 | III | 2 |
| 408 | GCA_001282035.1 | 562 | 562 | Escherichia coli | EPEC 1669 | Proteobacteria | LGBO01000067.1 | III | 2 |
| 409 | GCA_001282065.1 | 562 | 562 | Escherichia coli | STEC 2820 | Proteobacteria | LGBQ01000003.1 | III | 2 |
| 410 | GCA_001284665.1 | 562 | 562 | Escherichia coli | 503459_aEPEC | Proteobacteria | CXZO01000020.1 | III | 2 |
| 411 | GCA_001297965.1 | 562 | 562 | Escherichia coli | STEC 915 | Proteobacteria | LFUH01000101.1 | III | 2 |
| 412 | GCA_001297975.1 | 562 | 562 | Escherichia coli | STEC 343 | Proteobacteria | LDOZ01000007.1 | III | 2 |
| 413 | GCA_001297985.1 | 562 | 562 | Escherichia coli | STEC 623 | Proteobacteria | LFUB01000011.1 | III | 2 |
| 414 | GCA_001297995.1 | 562 | 562 | Escherichia coli | STEC 605 | Proteobacteria | LFUA01000020.1 | III | 2 |
| 415 | GCA_001309715.1 | 562 | 562 | Escherichia coli | PARC447 | Proteobacteria | LECJ01000192.1 | III | 2 |
| 416 | GCA_001309815.1 | 562 | 562 | Escherichia coli | 03-6430 | Proteobacteria | LEAB01000117.1 | III | 2 |
| 417 | GCA_001309835.1 | 562 | 562 | Escherichia coli | PARC449 | Proteobacteria | LEAC01000125.1 | III | 2 |
| 418 | GCA_001309885.1 | 562 | 562 | Escherichia coli | CO6CE900 | Proteobacteria | LEAE01000148.1 | III | 2 |
| 419 | GCA_001309895.1 | 562 | 562 | Escherichia coli | CO6CE1353 | Proteobacteria | LEAF01000141.1 | III | 2 |
| 420 | GCA_001309905.1 | 562 | 562 | Escherichia coli | CO6CE1943 | Proteobacteria | LEAG01000046.1 | III | 2 |
| 421 | GCA_001309925.1 | 562 | 562 | Escherichia coli | CO6CE2940 | Proteobacteria | LEAH01000084.1 | III | 2 |
| 422 | GCA_001309965.1 | 562 | 562 | Escherichia coli | CO283 | Proteobacteria | LEAI01000013.1 | III | 2 |
| 423 | GCA_001309985.1 | 562 | 562 | Escherichia coli | 1935 | Proteobacteria | LEAD01000163.1 | III | 2 |
| 424 | GCA_001309995.1 | 562 | 562 | Escherichia coli | E0122 | Proteobacteria | LEAJ01000145.1 | III | 2 |
| 425 | GCA_001310005.1 | 562 | 562 | Escherichia coli | LCDC7236 | Proteobacteria | LEAK01000079.1 | III | 2 |
| 426 | GCA_001484355.1 | 562 | 562 | Escherichia coli | AY71 | Proteobacteria | LLYJ01000063.1 | III | 2 |
| 427 | GCA_001484485.1 | 562 | 562 | Escherichia coli | AZ74 | Proteobacteria | LLYM01000074.1 | III | 2 |
| 428 | GCA_001518365.1 | 562 | 562 | Escherichia coli | GN02740 | Proteobacteria | LQZU01000211.1 | III | 2 |
| 429 | GCA_001518955.1 | 562 | 562 | Escherichia coli | SEC470 | Proteobacteria | CP013962.1 | III | 2 |
| 430 | GCA_001519395.1 | 562 | 562 | Escherichia coli | GN02270 | Proteobacteria | LQST01000002.1 | III | 2 |
| 431 | GCA_001519635.1 | 562 | 562 | Escherichia coli | GN02354 | Proteobacteria | LQTF01000057.1 | III | 2 |
| 432 | GCA_001519685.1 | 562 | 562 | Escherichia coli | GN02094 | Proteobacteria | LQSC01000049.1 | III | 2 |
| 433 | GCA_001519775.1 | 562 | 562 | Escherichia coli | GN02140 | Proteobacteria | LQSF01000099.1 | III | 2 |
| 434 | GCA_001519815.1 | 562 | 562 | Escherichia coli | GN02298 | Proteobacteria | LQSX01000125.1 | III | 2 |
| 435 | GCA_001519835.1 | 562 | 562 | Escherichia coli | GN02290 | Proteobacteria | LQSW01000149.1 | III | 2 |
| 436 | GCA_001519975.1 | 562 | 562 | Escherichia coli | GN02415 | Proteobacteria | LQTQ01000061.1 | III | 2 |
| 437 | GCA_001520115.1 | 562 | 562 | Escherichia coli | GN02343 | Proteobacteria | LQTC01000029.1 | III | 2 |
| 438 | GCA_001520255.1 | 562 | 562 | Escherichia coli | GN03578 | Proteobacteria | LQVZ01000203.1 | III | 2 |
| 439 | GCA_001520375.1 | 562 | 562 | Escherichia coli | GN02460 | Proteobacteria | LQTZ01000058.1 | III | 2 |
| 440 | GCA_001520435.1 | 562 | 562 | Escherichia coli | GN02430 | Proteobacteria | LQTU01000172.1 | III | 2 |
| 441 | GCA_001520495.1 | 562 | 562 | Escherichia coli | GN02426 | Proteobacteria | LQTS01000163.1 | III | 2 |
| 442 | GCA_001520575.1 | 562 | 562 | Escherichia coli | GN02674 | Proteobacteria | LQVA01000248.1 | III | 2 |
| 443 | GCA_001520835.1 | 562 | 562 | Escherichia coli | GN02707 | Proteobacteria | LQVE01000001.1 | III | 2 |
| 444 | GCA_001520935.1 | 562 | 562 | Escherichia coli | GN03218 | Proteobacteria | LQVQ01000300.1 | III | 2 |
| 445 | GCA_001520995.1 | 562 | 562 | Escherichia coli | GN02953 | Proteobacteria | LQVM01000164.1 | III | 2 |
| 446 | GCA_001521225.1 | 562 | 562 | Escherichia coli | GN02350 | Proteobacteria | LQTE01000128.1 | III | 2 |
| 447 | GCA_001521635.1 | 562 | 562 | Escherichia coli | GN04153 | Proteobacteria | LQWI01000295.1 | III | 2 |
| 448 | GCA_001521695.1 | 562 | 562 | Escherichia coli | GN03624 | Proteobacteria | LQWB01000001.1 | III | 2 |
| 449 | GCA_001558995.1 | 562 | 562 | Escherichia coli | JEONG-1266 | Proteobacteria | CP014314.1 | III | 2 |
| 450 | GCA_001562855.1 | 562 | 562 | Escherichia coli | JPH493 | Proteobacteria | KQ961400.1 | III | 2 |
| 451 | GCA_001571675.1 | 562 | 562 | Escherichia coli | UC5 | Proteobacteria | LRXT01000038.1 | III | 2 |
| 452 | GCA_001575305.1 | 562 | 562 | Escherichia coli | G19 | Proteobacteria | LONY01000024.1 | III | 2 |
| 453 | GCA_001575755.1 | 562 | 562 | Escherichia coli | G111 | Proteobacteria | LONJ01000019.1 | III | 2 |
| 454 | GCA_001575775.1 | 562 | 562 | Escherichia coli | G114 | Proteobacteria | LONK01000063.1 | III | 2 |
| 455 | GCA_001575885.1 | 562 | 562 | Escherichia coli | G169 | Proteobacteria | LONQ01000006.1 | III | 2 |
| 456 | GCA_001576035.1 | 562 | 562 | Escherichia coli | G27 | Proteobacteria | LOOY01000022.1 | III | 2 |
| 457 | GCA_001576105.1 | 562 | 562 | Escherichia coli | G314 | Proteobacteria | LOPH01000016.1 | III | 2 |
| 458 | GCA_001576135.1 | 562 | 562 | Escherichia coli | G313 | Proteobacteria | LOPG01000001.1 | III | 2 |
| 459 | GCA_001576325.1 | 562 | 562 | Escherichia coli | G28 | Proteobacteria | LOOZ010000011.1 | III | 2 |
| 460 | GCA_001576375.1 | 562 | 562 | Escherichia coli | G26 | Proteobacteria | LOOX01000037.1 | III | 2 |
| 461 | GCA_001576385.1 | 562 | 562 | Escherichia coli | G35 | Proteobacteria | LOPL01000002.1 | III | 2 |
| 462 | GCA_000303235.2 | 1005441 | 562 | Escherichia coli 0.1304 | 0.1304 | Proteobacteria | JH943332.1 | III | 2 |
| 463 | GCA_000462305.2 | 1081888 | 562 | Escherichia coli 08BKT055439 | 08BKT055439 | Proteobacteria | AVRI01000134.1 | III | 2 |
| 464 | GCA_000462325.2 | 1081895 | 562 | Escherichia coli 08BKT77219 | 08BKT77219 | Proteobacteria | AVRJ01000125.1 | III | 2 |
| 465 | GCA_000462345.2 | 1081894 | 562 | Escherichia coli 09BKT024447 | 09BKT024447 | Proteobacteria | AVRK01000010.1 | III | 2 |
| 466 | GCA_000461935.2 | 1081889 | 562 | Escherichia coli 09BKT076207 | 09BKT076207 | Proteobacteria | AVQR01000067.1 | III | 2 |
| 467 | GCA_000340455.2 | 1081887 | 562 | Escherichia coli 09BKT078844 | 09BKT078844 | Proteobacteria | KB453231.1 | III | 2 |
| 468 | GCA_000304855.2 | 1005469 | 562 | Escherichia coli 10.0821 | 10.0821 | Proteobacteria | AMTO01000004.1 | III | 2 |
| 469 | GCA_000305415.2 | 1005466 | 562 | Escherichia coli 10.0833 | 10.0833 | Proteobacteria | JH969098.1 | III | 2 |
| 470 | GCA_000305435.2 | 1005467 | 562 | Escherichia coli 10.0869 | 10.0869 | Proteobacteria | JH968727.1 | III | 2 |
| 471 | GCA_000601155.1 | 1444132 | 562 | Escherichia coli 1-110-08_S3_C1 | 1-110-08_S3_C1 | Proteobacteria | JHDN01000003.1 | III | 2 |
| 472 | GCA_000601175.1 | 1444160 | 562 | Escherichia coli 1-110-08_S3_C2 | 1-110-08_S3_C2 | Proteobacteria | JHDM01000091.1 | III | 2 |
| 473 | GCA_000601335.1 | 1444190 | 562 | Escherichia coli 1-110-08_S3_C3 | 1-110-08_S3_C3 | Proteobacteria | JHDL01000001.1 | III | 2 |
| 474 | GCA_000485635.1 | 1268976 | 562 | Escherichia coli 113290 | 113290 | Proteobacteria | KI521766.1 | III | 2 |
| 475 | GCA_000627435.1 | 1444135 | 562 | Escherichia coli 1-250-04_S3_C1 | 1-250-04_S3_C1 | Proteobacteria | JJLU01000053.1 | III | 2 |
| 476 | GCA_000713815.1 | 1444163 | 562 | Escherichia coli 1-250-04_S3_C2 | 1-250-04_S3_C2 | Proteobacteria | JOSR01000197.1 | III | 2 |
| 477 | GCA_000687105.1 | 1444147 | 562 | Escherichia coli 2-005-03_S3_C2 | 2-005-03_S3_C2 | Proteobacteria | JMGT01000075.1 | III | 2 |
| 478 | GCA_000700165.1 | 1444093 | 562 | Escherichia coli 2-011-08_S1_C3 | 2-011-08_S1_C3 | Proteobacteria | JNNA01000159.1 | III | 2 |
| 479 | GCA_000700605.1 | 1444119 | 562 | Escherichia coli 2-052-05_S3_C1 | 2-052-05_S3_C1 | Proteobacteria | JNNF01000074.1 | III | 2 |
| 480 | GCA_000713655.1 | 1444149 | 562 | Escherichia coli 2-052-05_S3_C2 | 2-052-05_S3_C2 | Proteobacteria | JOSJ01000098.1 | III | 2 |
| 481 | GCA_000703605.1 | 1444177 | 562 | Escherichia coli 2-052-05_S3_C3 | 2-052-05_S3_C3 | Proteobacteria | JNOZ01000070.1 | III | 2 |
| 482 | GCA_000703725.1 | 1444150 | 562 | Escherichia coli 2-156-04_S3_C2 | 2-156-04_S3_C2 | Proteobacteria | JNPK01000053.1 | III | 2 |
| 483 | GCA_000713695.1 | 1444123 | 562 | Escherichia coli 2-222-05_S3_C1 | 2-222-05_S3_C1 | Proteobacteria | JOSM01000267.1 | III | 2 |
| 484 | GCA_000713735.1 | 1444153 | 562 | Escherichia coli 2-222-05_S3_C2 | 2-222-05_S3_C2 | Proteobacteria | JOSN01000002.1 | III | 2 |
| 485 | GCA_000704145.1 | 1444181 | 562 | Escherichia coli 2-222-05_S3_C3 | 2-222-05_S3_C3 | Proteobacteria | JNQN01000045.1 | III | 2 |
| 486 | GCA_000711475.1 | 1444042 | 562 | Escherichia coli 2-316-03_S1_C1 | 2-316-03_S1_C1 | Proteobacteria | JOMW01000194.1 | III | 2 |
| 487 | GCA_000711415.1 | 1444071 | 562 | Escherichia coli 2-316-03_S1_C2 | 2-316-03_S1_C2 | Proteobacteria | JOMX01000159.1 | III | 2 |
| 488 | GCA_000355155.2 | 1116119 | 562 | Escherichia coli 2719100 | 2719100 | Proteobacteria | AQFG01000015.1 | III | 2 |
| 489 | GCA_000354775.2 | 1116010 | 562 | Escherichia coli 2747800 | 2747800 | Proteobacteria | AQEA01000085.1 | III | 2 |
| 490 | GCA_000354875.1 | 1116011 | 562 | Escherichia coli 2749250 | 2749250 | Proteobacteria | AQDZ01000107.1 | III | 2 |
| 491 | GCA_000355615.2 | 1116014 | 562 | Escherichia coli 2770900 | 2770900 | Proteobacteria | AQDW01000088.1 | III | 2 |
| 492 | GCA_000354995.2 | 1116021 | 562 | Escherichia coli 2860050 | 2860050 | Proteobacteria | AQDL01000093.1 | III | 2 |
| 493 | GCA_000356445.2 | 1116022 | 562 | Escherichia coli 2860650 | 2860650 | Proteobacteria | APXG01000173.1 | III | 2 |
| 494 | GCA_000356505.2 | 1116027 | 562 | Escherichia coli 2866350 | 2866350 | Proteobacteria | APXJ01000134.1 | III | 2 |
| 495 | GCA_000462245.2 | 1207038 | 562 | Escherichia coli 2886-75 | 2886-75 | Proteobacteria | AVRR01000001.1 | III | 2 |
| 496 | GCA_000304815.2 | 1005450 | 562 | Escherichia coli 3.4870 | 3.4870 | Proteobacteria | JH970627.1 | III | 2 |
| 497 | GCA_000335195.2 | 1051347 | 562 | Escherichia coli 3.4880 | 3.4880 | Proteobacteria | AOET01000122.1 | III | 2 |
| 498 | GCA_000700285.1 | 1444059 | 562 | Escherichia coli 3-475-03_S1_C1 | 3-475-03_S1_C1 | Proteobacteria | JNMJ01000048.1 | III | 2 |
| 499 | GCA_000713905.1 | 1444087 | 562 | Escherichia coli 3-475-03_S1_C2 | 3-475-03_S1_C2 | Proteobacteria | JOQC01000025.1 | III | 2 |
| 500 | GCA_000714055.1 | 1444171 | 562 | Escherichia coli 4-203-08_S3_C2 | 4-203-08_S3_C2 | Proteobacteria | JOQK01000042.1 | III | 2 |
| 501 | GCA_000713985.1 | 1444196 | 562 | Escherichia coli 4-203-08_S3_C3 | 4-203-08_S3_C3 | Proteobacteria | JOQH01000040.1 | III | 2 |
| 502 | GCA_000304875.2 | 1005452 | 562 | Escherichia coli 5.2239 | 5.2239 | Proteobacteria | JH970990.1 | III | 2 |
| 503 | GCA_000304055.2 | 1005561 | 562 | Escherichia coli 5412 | 5412 | Proteobacteria | JH954949.1 | III | 2 |
| 504 | GCA_000264135.1 | 752787 | 562 | Escherichia coli 576-1 | 576-1 | Proteobacteria | AJWS01000293.1 | III | 2 |
| 505 | GCA_000303995.2 | 1005558 | 562 | Escherichia coli 5905 | 5905 | Proteobacteria | JH958373.1 | III | 2 |
| 506 | GCA_000714385.1 | 1444226 | 562 | Escherichia coli 6-175-07_S4_C1 | 6-175-07_S4_C1 | Proteobacteria | JORB01000108.1 | III | 2 |
| 507 | GCA_000714415.1 | 1444255 | 562 | Escherichia coli 6-175-07_S4_C2 | 6-175-07_S4_C2 | Proteobacteria | JORC01000054.1 | III | 2 |
| 508 | GCA_000713175.1 | 1444281 | 562 | Escherichia coli 6-175-07_S4_C3 | 6-175-07_S4_C3 | Proteobacteria | JORK01000060.1 | III | 2 |
| 509 | GCA_000713335.1 | 1444062 | 562 | Escherichia coli 6-319-05_S1_C1 | 6-319-05_S1_C1 | Proteobacteria | JORT01000068.1 | III | 2 |
| 510 | GCA_000713025.1 | 1444090 | 562 | Escherichia coli 6-319-05_S1_C2 | 6-319-05_S1_C2 | Proteobacteria | JORD01000183.1 | III | 2 |
| 511 | GCA_000713015.1 | 1444115 | 562 | Escherichia coli 6-319-05_S1_C3 | 6-319-05_S1_C3 | Proteobacteria | JORE01000064.1 | III | 2 |
| 512 | GCA_000713255.1 | 1444227 | 562 | Escherichia coli 6-537-08_S4_C1 | 6-537-08_S4_C1 | Proteobacteria | JORS01000112.1 | III | 2 |
| 513 | GCA_000713605.1 | 1444257 | 562 | Escherichia coli 6-537-08_S4_C2 | 6-537-08_S4_C2 | Proteobacteria | JOSG01000027.1 | III | 2 |
| 514 | GCA_000335115.1 | 1051348 | 562 | Escherichia coli 7.1982 | 7.1982 | Proteobacteria | AOEP01000011.1 | III | 2 |
| 515 | GCA_000304835.2 | 1005463 | 562 | Escherichia coli 8.0416 | 8.0416 | Proteobacteria | AMTN01000133.1 | III | 2 |
| 516 | GCA_000305375.2 | 1005461 | 562 | Escherichia coli 8.0586 | 8.0586 | Proteobacteria | JH969648.1 | III | 2 |
| 517 | GCA_000305395.2 | 1005460 | 562 | Escherichia coli 8.2524 | 8.2524 | Proteobacteria | JH969044.1 | III | 2 |
| 518 | GCA_000305455.2 | 1005397 | 562 | Escherichia coli 88.0221 | 88.0221 | Proteobacteria | JH968005.1 | III | 2 |
| 519 | GCA_000316345.2 | 1005398 | 562 | Escherichia coli 88.1042 | 88.1042 | Proteobacteria | KB006081.1 | III | 2 |
| 520 | GCA_000316365.2 | 1005399 | 562 | Escherichia coli 89.0511 | 89.0511 | Proteobacteria | KB007266.1 | III | 2 |
| 521 | GCA_000316385.2 | 1005400 | 562 | Escherichia coli 90.0091 | 90.0091 | Proteobacteria | KB007294.1 | III | 2 |
| 522 | GCA_000316425.2 | 1005402 | 562 | Escherichia coli 90.2281 | 90.2281 | Proteobacteria | KB005715.1 | III | 2 |
| 523 | GCA_000488355.1 | 1268982 | 562 | Escherichia coli 907672 | 907672 | Proteobacteria | KI531258.1 | III | 2 |
| 524 | GCA_000485695.1 | 1268986 | 562 | Escherichia coli 907713 | 907713 | Proteobacteria | KI522246.1 | III | 2 |
| 525 | GCA_000488435.1 | 1268987 | 562 | Escherichia coli 907715 | 907715 | Proteobacteria | KI531882.1 | III | 2 |
| 526 | GCA_000488515.1 | 1268992 | 562 | Escherichia coli 908521 | 908521 | Proteobacteria | KI532686.1 | III | 2 |
| 527 | GCA_000488535.1 | 1268993 | 562 | Escherichia coli 908522 | 908522 | Proteobacteria | KI532821.1 | III | 2 |
| 528 | GCA_000488575.1 | 1268995 | 562 | Escherichia coli 908525 | 908525 | Proteobacteria | KI533062.1 | III | 2 |
| 529 | GCA_000488655.1 | 1268999 | 562 | Escherichia coli 908585 | 908585 | Proteobacteria | KI533791.1 | III | 2 |
| 530 | GCA_000488675.1 | 1269000 | 562 | Escherichia coli 908616 | 908616 | Proteobacteria | KI533940.1 | III | 2 |
| 531 | GCA_000316445.2 | 1005403 | 562 | Escherichia coli 93.0055 | 93.0055 | Proteobacteria | KB004131.1 | III | 2 |
| 532 | GCA_000316465.1 | 1005404 | 562 | Escherichia coli 93.0056 | 93.0056 | Proteobacteria | KB004997.1 | III | 2 |
| 533 | GCA_000267945.2 | 1005481 | 562 | Escherichia coli 93-001 | 93-001 | Proteobacteria | JH710997.1 | III | 2 |
| 534 | GCA_000316485.1 | 1005405 | 562 | Escherichia coli 94.0618 | 94.0618 | Proteobacteria | KB005707.1 | III | 2 |
| 535 | GCA_000335375.2 | 1051346 | 562 | Escherichia coli 95.0083 | 95.0083 | Proteobacteria | AOEU01000192.1 | III | 2 |
| 536 | GCA_000316865.2 | 1005407 | 562 | Escherichia coli 95.1288 | 95.1288 | Proteobacteria | KB001712.1 | III | 2 |
| 537 | GCA_000316765.2 | 1005415 | 562 | Escherichia coli 96.0107 | 96.0107 | Proteobacteria | KB003449.1 | III | 2 |
| 538 | GCA_000318425.2 | 1005414 | 562 | Escherichia coli 96.0109 | 96.0109 | Proteobacteria | KB009454.1 | III | 2 |
| 539 | GCA_000316825.2 | 1005411 | 562 | Escherichia coli 96.0427 | 96.0427 | Proteobacteria | KB002687.1 | III | 2 |
| 540 | GCA_000316905.2 | 1005410 | 562 | Escherichia coli 96.0428 | 96.0428 | Proteobacteria | KB000504.1 | III | 2 |
| 541 | GCA_000316805.2 | 1005413 | 562 | Escherichia coli 96.0932 | 96.0932 | Proteobacteria | KB003092.1 | III | 2 |
| 542 | GCA_000316745.2 | 1005416 | 562 | Escherichia coli 97.0003 | 97.0003 | Proteobacteria | JH999738.1 | III | 2 |
| 543 | GCA_000316505.2 | 1005420 | 562 | Escherichia coli 97.0007 | 97.0007 | Proteobacteria | ANMC01000275.1 | III | 2 |
| 544 | GCA_000318445.2 | 1005419 | 562 | Escherichia coli 97.0010 | 97.0010 | Proteobacteria | KB008878.1 | III | 2 |
| 545 | GCA_000316725.2 | 1005418 | 562 | Escherichia coli 97.1742 | 97.1742 | Proteobacteria | KB004341.1 | III | 2 |
| 546 | GCA_000335215.2 | 1005423 | 562 | Escherichia coli 99.0670 | 99.0670 | Proteobacteria | AOEV01000013.1 | III | 2 |
| 547 | GCA_000316545.2 | 1005422 | 562 | Escherichia coli 99.0678 | 99.0678 | Proteobacteria | ANME01000013.1 | III | 2 |
| 548 | GCA_000316565.2 | 1005424 | 562 | Escherichia coli 99.0713 | 99.0713 | Proteobacteria | ANMF01000001.1 | III | 2 |
| 549 | GCA_000335255.2 | 1005432 | 562 | Escherichia coli 99.0839 | 99.0839 | Proteobacteria | AOEB01000180.1 | III | 2 |
| 550 | GCA_000335275.2 | 1005431 | 562 | Escherichia coli 99.0848 | 99.0848 | Proteobacteria | AOEC01000227.1 | III | 2 |
| 551 | GCA_000335295.2 | 1005440 | 562 | Escherichia coli 99.1753 | 99.1753 | Proteobacteria | AOED01000248.1 | III | 2 |
| 552 | GCA_000335155.2 | 1005435 | 562 | Escherichia coli 99.1762 | 99.1762 | Proteobacteria | AOER01000006.1 | III | 2 |
| 553 | GCA_000335315.2 | 1005438 | 562 | Escherichia coli 99.1775 | 99.1775 | Proteobacteria | AOEE01000251.1 | III | 2 |
| 554 | GCA_000335135.1 | 1005439 | 562 | Escherichia coli 99.1781 | 99.1781 | Proteobacteria | AOEQ01000012.1 | III | 2 |
| 555 | GCA_000335015.2 | 1005437 | 562 | Escherichia coli 99.1793 | 99.1793 | Proteobacteria | AOEF01000014.1 | III | 2 |
| 556 | GCA_000473725.2 | 1389955 | 562 | Escherichia coli ATCC 35150 | ATCC 35150 | Proteobacteria | AWXM02000035.1 | III | 2 |
| 557 | GCA_000335055.2 | 1051353 | 562 | Escherichia coli ATCC 700728 | ATCC 700728 | Proteobacteria | AOEH01000126.1 | III | 2 |
| 558 | GCA_000019385.1 | 481805 | 562 | Escherichia coli ATCC 8739 | ATCC 8739 | Proteobacteria | CP000946.1 | III | 2 |
| 559 | GCA_000496345.2 | 1404261 | 562 | Escherichia coli ATCC BAA-2192 | ATCC BAA-2192 | Proteobacteria | AYGW02000190.1 | III | 2 |
| 560 | GCA_000461955.2 | 1151213 | 562 | Escherichia coli B102 | B102 | Proteobacteria | AVQS01000008.1 | III | 2 |
| 561 | GCA_000462485.2 | 1151234 | 562 | Escherichia coli B103 | B103 | Proteobacteria | AVRS01000001.1 | III | 2 |
| 562 | GCA_000462505.2 | 1151243 | 562 | Escherichia coli B104 | B104 | Proteobacteria | AVRT01000005.1 | III | 2 |
| 563 | GCA_000462525.2 | 1151247 | 562 | Escherichia coli B105 | B105 | Proteobacteria | AVRU01000005.1 | III | 2 |
| 564 | GCA_000462545.2 | 1151248 | 562 | Escherichia coli B106 | B106 | Proteobacteria | AVRV01000001.1 | III | 2 |
| 565 | GCA_000461995.2 | 1151214 | 562 | Escherichia coli B107 | B107 | Proteobacteria | AVQT01000005.1 | III | 2 |
| 566 | GCA_000462565.2 | 550673 | 562 | Escherichia coli B108 | B108 | Proteobacteria | AVRW01000007.1 | III | 2 |
| 567 | GCA_000462585.2 | 1151244 | 562 | Escherichia coli B109 | B109 | Proteobacteria | AVRX01000001.1 | III | 2 |
| 568 | GCA_000462605.2 | 1151249 | 562 | Escherichia coli B112 | B112 | Proteobacteria | AVRY01000010.1 | III | 2 |
| 569 | GCA_000462625.2 | 1151250 | 562 | Escherichia coli B113 | B113 | Proteobacteria | AVRZ01000133.1 | III | 2 |
| 570 | GCA_000462645.2 | 1151251 | 562 | Escherichia coli B114 | B114 | Proteobacteria | AVSA01000001.1 | III | 2 |
| 571 | GCA_000462185.2 | 1151237 | 562 | Escherichia coli B15 | B15 | Proteobacteria | AVSB01000112.1 | III | 2 |
| 572 | GCA_000462665.2 | 1151238 | 562 | Escherichia coli B17 | B17 | Proteobacteria | AVSC01000121.1 | III | 2 |
| 573 | GCA_000462025.2 | 1151225 | 562 | Escherichia coli B26-1 | B26-1 | Proteobacteria | AVQU01000001.1 | III | 2 |
| 574 | GCA_000461915.2 | 1151226 | 562 | Escherichia coli B26-2 | B26-2 | Proteobacteria | AVQV01000001.1 | III | 2 |
| 575 | GCA_000462045.2 | 1151217 | 562 | Escherichia coli B28-1 | B28-1 | Proteobacteria | AVQW01000010.1 | III | 2 |
| 576 | GCA_000477495.2 | 1151218 | 562 | Escherichia coli B28-2 | B28-2 | Proteobacteria | AVQX01000012.1 | III | 2 |
| 577 | GCA_000461975.2 | 1151219 | 562 | Escherichia coli B29-1 | B29-1 | Proteobacteria | AVQY01000001.1 | III | 2 |
| 578 | GCA_000462105.2 | 1151220 | 562 | Escherichia coli B29-2 | B29-2 | Proteobacteria | AVQZ01000006.1 | III | 2 |
| 579 | GCA_000462125.2 | 1151221 | 562 | Escherichia coli B36-1 | B36-1 | Proteobacteria | AVRA01000107.1 | III | 2 |
| 580 | GCA_000462065.2 | 1151222 | 562 | Escherichia coli B36-2 | B36-2 | Proteobacteria | AVRB01000001.1 | III | 2 |
| 581 | GCA_000462685.2 | 1151227 | 562 | Escherichia coli B40-1 | B40-1 | Proteobacteria | AVSD01000010.1 | III | 2 |
| 582 | GCA_000462705.2 | 1151228 | 562 | Escherichia coli B40-2 | B40-2 | Proteobacteria | AVSE01000003.1 | III | 2 |
| 583 | GCA_000462725.2 | 1151246 | 562 | Escherichia coli B49-2 | B49-2 | Proteobacteria | AVSF01000006.1 | III | 2 |
| 584 | GCA_000462745.2 | 1151224 | 562 | Escherichia coli B5-2 | B5-2 | Proteobacteria | AVSG01000001.1 | III | 2 |
| 585 | GCA_000462085.2 | 1151215 | 562 | Escherichia coli B7-1 | B7-1 | Proteobacteria | AVRC01000049.1 | III | 2 |
| 586 | GCA_000462405.2 | 1151216 | 562 | Escherichia coli B7-2 | B7-2 | Proteobacteria | AVRD01000050.1 | III | 2 |
| 587 | GCA_000462765.2 | 1151239 | 562 | Escherichia coli B83 | B83 | Proteobacteria | AVSH01000001.1 | III | 2 |
| 588 | GCA_000462785.2 | 1151240 | 562 | Escherichia coli B84 | B84 | Proteobacteria | AVSI01000012.1 | III | 2 |
| 589 | GCA_000462805.2 | 1151241 | 562 | Escherichia coli B85 | B85 | Proteobacteria | AVSJ01000007.1 | III | 2 |
| 590 | GCA_000462825.2 | 1151242 | 562 | Escherichia coli B86 | B86 | Proteobacteria | AVSK01000136.1 | III | 2 |
| 591 | GCA_000462845.2 | 1151235 | 562 | Escherichia coli B89 | B89 | Proteobacteria | AVSL01000001.1 | III | 2 |
| 592 | GCA_000462865.2 | 1151236 | 562 | Escherichia coli B90 | B90 | Proteobacteria | AVSM01000129.1 | III | 2 |
| 593 | GCA_000462425.2 | 1151231 | 562 | Escherichia coli B93 | B93 | Proteobacteria | AVRE01000046.1 | III | 2 |
| 594 | GCA_000462445.2 | 1151232 | 562 | Escherichia coli B94 | B94 | Proteobacteria | AVRF01000052.1 | III | 2 |
| 595 | GCA_000462465.2 | 1151233 | 562 | Escherichia coli B95 | B95 | Proteobacteria | AVRG01000053.1 | III | 2 |
| 596 | GCA_000522245.1 | 1328440 | 562 | Escherichia coli BIDMC 15 | BIDMC 15 | Proteobacteria | KI929736.1 | III | 2 |
| 597 | GCA_000522225.1 | 1328441 | 562 | Escherichia coli BIDMC 17A | BIDMC 17A | Proteobacteria | KI929720.1 | III | 2 |
| 598 | GCA_000522205.1 | 1328442 | 562 | Escherichia coli BIDMC 17B | BIDMC 17B | Proteobacteria | KI929706.1 | III | 2 |
| 599 | GCA_000522185.1 | 1328443 | 562 | Escherichia coli BIDMC 19A | BIDMC 19A | Proteobacteria | KI929698.1 | III | 2 |
| 600 | GCA_000522165.1 | 1328444 | 562 | Escherichia coli BIDMC 19B | BIDMC 19B | Proteobacteria | KI929691.1 | III | 2 |
| 601 | GCA_000474825.1 | 1328445 | 562 | Escherichia coli BIDMC 19C | BIDMC 19C | Proteobacteria | AXLI01000001.1 | III | 2 |
| 602 | GCA_000522305.1 | 1328436 | 562 | Escherichia coli BIDMC 2B | BIDMC 2B | Proteobacteria | KI929775.1 | III | 2 |
| 603 | GCA_000522285.1 | 1328437 | 562 | Escherichia coli BIDMC 3 | BIDMC 3 | Proteobacteria | KI929768.1 | III | 2 |
| 604 | GCA_000492235.1 | 1328450 | 562 | Escherichia coli BIDMC 39 | BIDMC 39 | Proteobacteria | KI535383.1 | III | 2 |
| 605 | GCA_000522105.1 | 1400156 | 562 | Escherichia coli BIDMC 43a | BIDMC 43a | Proteobacteria | KI929669.1 | III | 2 |
| 606 | GCA_000522085.1 | 1400157 | 562 | Escherichia coli BIDMC 43b | BIDMC 43b | Proteobacteria | KI929660.1 | III | 2 |
| 607 | GCA_000522405.1 | 1328438 | 562 | Escherichia coli BIDMC 6 | BIDMC 6 | Proteobacteria | KI929808.1 | III | 2 |
| 608 | GCA_000633675.1 | 1445862 | 562 | Escherichia coli BIDMC 82 | BIDMC 82 | Proteobacteria | KK214161.1 | III | 2 |
| 609 | GCA_000522265.1 | 1328439 | 562 | Escherichia coli BIDMC 9 | BIDMC 9 | Proteobacteria | KI929754.1 | III | 2 |
| 610 | GCA_000304015.2 | 1005559 | 562 | Escherichia coli CB7326 | CB7326 | Proteobacteria | JH956766.1 | III | 2 |
| 611 | GCA_000731355.1 | 1400022 | 562 | Escherichia coli D6-113.11 | D6-113.11 | Proteobacteria | HG977177.1 | III | 2 |
| 612 | GCA_000249495.2 | 868155 | 562 | Escherichia coli DEC5A | DEC5A | Proteobacteria | AIFQ01000037.1 | III | 2 |
| 613 | GCA_000267045.1 | 1005536 | 562 | Escherichia coli EC1734 | EC1734 | Proteobacteria | AKMO01000001.1 | III | 2 |
| 614 | GCA_000303615.2 | 1005537 | 562 | Escherichia coli EC1735 | EC1735 | Proteobacteria | JH958312.1 | III | 2 |
| 615 | GCA_000303595.2 | 1005538 | 562 | Escherichia coli EC1736 | EC1736 | Proteobacteria | JH965238.1 | III | 2 |
| 616 | GCA_000303575.2 | 1005539 | 562 | Escherichia coli EC1737 | EC1737 | Proteobacteria | JH963529.1 | III | 2 |
| 617 | GCA_000267025.1 | 1005535 | 562 | Escherichia coli EC1738 | EC1738 | Proteobacteria | AKMN01000003.1 | III | 2 |
| 618 | GCA_000267225.2 | 1005540 | 562 | Escherichia coli EC1845 | EC1845 | Proteobacteria | JH693448.1 | III | 2 |
| 619 | GCA_000303555.2 | 1005541 | 562 | Escherichia coli EC1846 | EC1846 | Proteobacteria | JH956315.1 | III | 2 |
| 620 | GCA_000303535.2 | 1005542 | 562 | Escherichia coli EC1847 | EC1847 | Proteobacteria | JH963080.1 | III | 2 |
| 621 | GCA_000303515.2 | 1005543 | 562 | Escherichia coli EC1848 | EC1848 | Proteobacteria | JH955442.1 | III | 2 |
| 622 | GCA_000303495.2 | 1005544 | 562 | Escherichia coli EC1849 | EC1849 | Proteobacteria | JH953696.1 | III | 2 |
| 623 | GCA_000303475.2 | 1005545 | 562 | Escherichia coli EC1850 | EC1850 | Proteobacteria | JH952886.1 | III | 2 |
| 624 | GCA_000303455.2 | 1005546 | 562 | Escherichia coli EC1856 | EC1856 | Proteobacteria | JH952880.1 | III | 2 |
| 625 | GCA_000303435.2 | 1005547 | 562 | Escherichia coli EC1862 | EC1862 | Proteobacteria | JH952466.1 | III | 2 |
| 626 | GCA_000267205.2 | 1005548 | 562 | Escherichia coli EC1863 | EC1863 | Proteobacteria | JH692972.1 | III | 2 |
| 627 | GCA_000303415.2 | 1005549 | 562 | Escherichia coli EC1864 | EC1864 | Proteobacteria | JH945870.1 | III | 2 |
| 628 | GCA_000303375.2 | 1005551 | 562 | Escherichia coli EC1866 | EC1866 | Proteobacteria | JH944315.1 | III | 2 |
| 629 | GCA_000303355.2 | 1005552 | 562 | Escherichia coli EC1868 | EC1868 | Proteobacteria | JH944350.1 | III | 2 |
| 630 | GCA_000303335.2 | 1005553 | 562 | Escherichia coli EC1869 | EC1869 | Proteobacteria | JH946714.1 | III | 2 |
| 631 | GCA_000303315.2 | 1005554 | 562 | Escherichia coli EC1870 | EC1870 | Proteobacteria | JH945214.1 | III | 2 |
| 632 | GCA_000267345.2 | 1005529 | 562 | Escherichia coli EC4013 | EC4013 | Proteobacteria | JH698305.1 | III | 2 |
| 633 | GCA_000267445.2 | 1005524 | 562 | Escherichia coli EC4196 | EC4196 | Proteobacteria | JH699193.1 | III | 2 |
| 634 | GCA_000267465.2 | 1005523 | 562 | Escherichia coli EC4203 | EC4203 | Proteobacteria | JH699677.1 | III | 2 |
| 635 | GCA_000267325.1 | 1005530 | 562 | Escherichia coli EC4402 | EC4402 | Proteobacteria | JH697728.1 | III | 2 |
| 636 | GCA_000267385.2 | 1005527 | 562 | Escherichia coli EC4421 | EC4421 | Proteobacteria | JH696600.1 | III | 2 |
| 637 | GCA_000267365.2 | 1005528 | 562 | Escherichia coli EC4422 | EC4422 | Proteobacteria | JH695870.1 | III | 2 |
| 638 | GCA_000267285.2 | 1005532 | 562 | Escherichia coli EC4436 | EC4436 | Proteobacteria | JH694746.1 | III | 2 |
| 639 | GCA_000267265.2 | 1005533 | 562 | Escherichia coli EC4437 | EC4437 | Proteobacteria | JH694340.1 | III | 2 |
| 640 | GCA_000267305.2 | 1005531 | 562 | Escherichia coli EC4439 | EC4439 | Proteobacteria | JH697080.1 | III | 2 |
| 641 | GCA_000267245.2 | 1005534 | 562 | Escherichia coli EC4448 | EC4448 | Proteobacteria | JH693935.1 | III | 2 |
| 642 | GCA_000304035.2 | 1005560 | 562 | Escherichia coli EC96038 | EC96038 | Proteobacteria | JH955436.1 | III | 2 |
| 643 | GCA_000498235.1 | 758832 | 562 | Escherichia coli ECC-Z | ECC-Z | Proteobacteria | AHHN01000002.1 | III | 2 |
| 644 | GCA_000355195.2 | 1115998 | 562 | Escherichia coli Envira 10/1 | Envira 10/1 | Proteobacteria | AQFD01000020.1 | III | 2 |
| 645 | GCA_000355215.2 | 1115997 | 562 | Escherichia coli Envira 8/11 | Envira 8/11 | Proteobacteria | AQFC01000021.1 | III | 2 |
| 646 | GCA_000303755.2 | 1005470 | 562 | Escherichia coli FDA504 | FDA504 | Proteobacteria | JH961871.1 | III | 2 |
| 647 | GCA_000267005.2 | 1005471 | 562 | Escherichia coli FDA505 | FDA505 | Proteobacteria | JH692596.1 | III | 2 |
| 648 | GCA_000303715.2 | 1005474 | 562 | Escherichia coli FDA506 | FDA506 | Proteobacteria | JH964536.1 | III | 2 |
| 649 | GCA_000303735.2 | 1005472 | 562 | Escherichia coli FDA507 | FDA507 | Proteobacteria | JH963945.1 | III | 2 |
| 650 | GCA_000267485.2 | 1005473 | 562 | Escherichia coli FDA517 | FDA517 | Proteobacteria | JH701995.1 | III | 2 |
| 651 | GCA_000267525.2 | 1005476 | 562 | Escherichia coli FRIK1985 | FRIK1985 | Proteobacteria | JH701533.1 | III | 2 |
| 652 | GCA_000303795.2 | 1005480 | 562 | Escherichia coli FRIK1997 | FRIK1997 | Proteobacteria | JH961362.1 | III | 2 |
| 653 | GCA_000303275.2 | 1005482 | 562 | Escherichia coli FRIK523 | FRIK523 | Proteobacteria | JH943936.1 | III | 2 |
| 654 | GCA_000163215.1 | 656379 | 562 | Escherichia coli FVEC1302 | FVEC1302 | Proteobacteria | GG774899.1 | III | 2 |
| 655 | GCA_000163235.1 | 656380 | 562 | Escherichia coli FVEC1412 | FVEC1412 | Proteobacteria | GG749206.1 | III | 2 |
| 656 | GCA_000456165.1 | 1280964 | 562 | Escherichia coli HVH 10 (4-6832164) | HVH 10 (4-6832164) | Proteobacteria | KE698889.1 | III | 2 |
| 657 | GCA_000457475.1 | 1281042 | 562 | Escherichia coli HVH 106 (4-6881831) | HVH 106 (4-6881831) | Proteobacteria | KE699746.1 | III | 2 |
| 658 | GCA_000457535.1 | 1281046 | 562 | Escherichia coli HVH 110 (4-6978754) | HVH 110 (4-6978754) | Proteobacteria | KE699792.1 | III | 2 |
| 659 | GCA_000457595.1 | 1281049 | 562 | Escherichia coli HVH 113 (4-7535473) | HVH 113 (4-7535473) | Proteobacteria | KE699840.1 | III | 2 |
| 660 | GCA_000457735.1 | 1281056 | 562 | Escherichia coli HVH 119 (4-6879578) | HVH 119 (4-6879578) | Proteobacteria | KE699940.1 | III | 2 |
| 661 | GCA_000457795.1 | 1281059 | 562 | Escherichia coli HVH 122 (4-6851606) | HVH 122 (4-6851606) | Proteobacteria | KE699976.1 | III | 2 |
| 662 | GCA_000457895.1 | 1281064 | 562 | Escherichia coli HVH 130 (4-7036876) | HVH 130 (4-7036876) | Proteobacteria | KE700051.1 | III | 2 |
| 663 | GCA_000457955.1 | 1281067 | 562 | Escherichia coli HVH 134 (4-6073441) | HVH 134 (4-6073441) | Proteobacteria | KE700096.1 | III | 2 |
| 664 | GCA_000457975.1 | 1281068 | 562 | Escherichia coli HVH 135 (4-4449320) | HVH 135 (4-4449320) | Proteobacteria | KE700100.1 | III | 2 |
| 665 | GCA_000494995.1 | 1281069 | 562 | Escherichia coli HVH 136 (4-5970458) | HVH 136 (4-5970458) | Proteobacteria | KI538714.1 | III | 2 |
| 666 | GCA_000458055.1 | 1281073 | 562 | Escherichia coli HVH 140 (4-5894387) | HVH 140 (4-5894387) | Proteobacteria | KE700176.1 | III | 2 |
| 667 | GCA_000458155.1 | 1281078 | 562 | Escherichia coli HVH 145 (4-5672112) | HVH 145 (4-5672112) | Proteobacteria | KE700231.1 | III | 2 |
| 668 | GCA_000458235.1 | 1281083 | 562 | Escherichia coli HVH 150 (4-3258106) | HVH 150 (4-3258106) | Proteobacteria | KE700264.1 | III | 2 |
| 669 | GCA_000458255.1 | 1281084 | 562 | Escherichia coli HVH 151 (4-5755573) | HVH 151 (4-5755573) | Proteobacteria | KE700275.1 | III | 2 |
| 670 | GCA_000458315.1 | 1281088 | 562 | Escherichia coli HVH 155 (4-4509048) | HVH 155 (4-4509048) | Proteobacteria | KE700348.1 | III | 2 |
| 671 | GCA_000458475.1 | 1281096 | 562 | Escherichia coli HVH 163 (4-4697553) | HVH 163 (4-4697553) | Proteobacteria | KE700462.1 | III | 2 |
| 672 | GCA_000458515.1 | 1281098 | 562 | Escherichia coli HVH 167 (4-6073565) | HVH 167 (4-6073565) | Proteobacteria | KE700479.1 | III | 2 |
| 673 | GCA_000458625.1 | 1281103 | 562 | Escherichia coli HVH 173 (3-9175482) | HVH 173 (3-9175482) | Proteobacteria | KE700542.1 | III | 2 |
| 674 | GCA_000458705.1 | 1281109 | 562 | Escherichia coli HVH 182 (4-0985554) | HVH 182 (4-0985554) | Proteobacteria | KE700608.1 | III | 2 |
| 675 | GCA_000458725.1 | 1281110 | 562 | Escherichia coli HVH 183 (4-3205932) | HVH 183 (4-3205932) | Proteobacteria | KE700619.1 | III | 2 |
| 676 | GCA_000458915.1 | 1281120 | 562 | Escherichia coli HVH 193 (4-3331423) | HVH 193 (4-3331423) | Proteobacteria | KE700785.1 | III | 2 |
| 677 | GCA_000458995.1 | 1281124 | 562 | Escherichia coli HVH 197 (4-4466217) | HVH 197 (4-4466217) | Proteobacteria | KE700814.1 | III | 2 |
| 678 | GCA_000459175.1 | 1281133 | 562 | Escherichia coli HVH 206 (4-3128229) | HVH 206 (4-3128229) | Proteobacteria | KE700892.1 | III | 2 |
| 679 | GCA_000459215.1 | 1281135 | 562 | Escherichia coli HVH 208 (4-3112292) | HVH 208 (4-3112292) | Proteobacteria | KE700918.1 | III | 2 |
| 680 | GCA_000459335.1 | 1281142 | 562 | Escherichia coli HVH 215 (4-3008371) | HVH 215 (4-3008371) | Proteobacteria | KE700987.1 | III | 2 |
| 681 | GCA_000456325.1 | 1280973 | 562 | Escherichia coli HVH 22 (4-2258986) | HVH 22 (4-2258986) | Proteobacteria | KE699031.1 | III | 2 |
| 682 | GCA_000459475.1 | 1281149 | 562 | Escherichia coli HVH 223 (4-2976528) | HVH 223 (4-2976528) | Proteobacteria | KE701095.1 | III | 2 |
| 683 | GCA_000456445.1 | 1280980 | 562 | Escherichia coli HVH 29 (4-3418073) | HVH 29 (4-3418073) | Proteobacteria | KE699106.1 | III | 2 |
| 684 | GCA_000456685.1 | 1280993 | 562 | Escherichia coli HVH 43 (4-2173468) | HVH 43 (4-2173468) | Proteobacteria | KE699249.1 | III | 2 |
| 685 | GCA_000456705.1 | 1280994 | 562 | Escherichia coli HVH 44 (4-2298570) | HVH 44 (4-2298570) | Proteobacteria | KE699258.1 | III | 2 |
| 686 | GCA_000456725.1 | 1280995 | 562 | Escherichia coli HVH 45 (4-3129918) | HVH 45 (4-3129918) | Proteobacteria | KE699267.1 | III | 2 |
| 687 | GCA_000456925.1 | 1281007 | 562 | Escherichia coli HVH 63 (4-2542528) | HVH 63 (4-2542528) | Proteobacteria | KE699373.1 | III | 2 |
| 688 | GCA_000456985.1 | 1281010 | 562 | Escherichia coli HVH 69 (4-2837072) | HVH 69 (4-2837072) | Proteobacteria | KE699403.1 | III | 2 |
| 689 | GCA_000457205.1 | 1281022 | 562 | Escherichia coli HVH 85 (4-0792144) | HVH 85 (4-0792144) | Proteobacteria | KE699590.1 | III | 2 |
| 690 | GCA_000459655.1 | 1281158 | 562 | Escherichia coli KOEGE 33 (68a) | KOEGE 33 (68a) | Proteobacteria | KE701249.1 | III | 2 |
| 691 | GCA_000459795.1 | 1281165 | 562 | Escherichia coli KOEGE 62 (175a) | KOEGE 62 (175a) | Proteobacteria | KE701351.1 | III | 2 |
| 692 | GCA_000459815.1 | 1281166 | 562 | Escherichia coli KOEGE 68 (182a) | KOEGE 68 (182a) | Proteobacteria | KE701357.1 | III | 2 |
| 693 | GCA_000459875.1 | 1281169 | 562 | Escherichia coli KOEGE 73 (195a) | KOEGE 73 (195a) | Proteobacteria | KE701395.1 | III | 2 |
| 694 | GCA_000407665.1 | 1169315 | 562 | Escherichia coli KTE1 | KTE1 | Proteobacteria | KE136571.1 | III | 2 |
| 695 | GCA_000408585.1 | 1182701 | 562 | Escherichia coli KTE102 | KTE102 | Proteobacteria | KE137162.1 | III | 2 |
| 696 | GCA_000351885.1 | 1182708 | 562 | Escherichia coli KTE116 | KTE116 | Proteobacteria | KB732889.1 | III | 2 |
| 697 | GCA_000326945.1 | 1169380 | 562 | Escherichia coli KTE122 | KTE122 | Proteobacteria | KB731963.1 | III | 2 |
| 698 | GCA_000326965.1 | 1169382 | 562 | Escherichia coli KTE125 | KTE125 | Proteobacteria | KB732191.1 | III | 2 |
| 699 | GCA_000408705.1 | 1182714 | 562 | Escherichia coli KTE127 | KTE127 | Proteobacteria | KE137233.1 | III | 2 |
| 700 | GCA_000326245.1 | 1169383 | 562 | Escherichia coli KTE128 | KTE128 | Proteobacteria | ANXW01000117.1 | III | 2 |
| 701 | GCA_000408725.1 | 1182715 | 562 | Escherichia coli KTE130 | KTE130 | Proteobacteria | KE137242.1 | III | 2 |
| 702 | GCA_000408745.1 | 1182716 | 562 | Escherichia coli KTE132 | KTE132 | Proteobacteria | KE137261.1 | III | 2 |
| 703 | GCA_000408765.1 | 1182717 | 562 | Escherichia coli KTE134 | KTE134 | Proteobacteria | KE137276.1 | III | 2 |
| 704 | GCA_000352725.1 | 1182728 | 562 | Escherichia coli KTE154 | KTE154 | Proteobacteria | KB733130.1 | III | 2 |
| 705 | GCA_000352745.1 | 1182731 | 562 | Escherichia coli KTE158 | KTE158 | Proteobacteria | KB733136.1 | III | 2 |
| 706 | GCA_000351985.1 | 1182733 | 562 | Escherichia coli KTE161 | KTE161 | Proteobacteria | KB732934.1 | III | 2 |
| 707 | GCA_000326765.1 | 1169397 | 562 | Escherichia coli KTE166 | KTE166 | Proteobacteria | ANYK01000003.1 | III | 2 |
| 708 | GCA_000408825.1 | 1182737 | 562 | Escherichia coli KTE170 | KTE170 | Proteobacteria | KE137303.1 | III | 2 |
| 709 | GCA_000350925.1 | 1181727 | 562 | Escherichia coli KTE181 | KTE181 | Proteobacteria | KB732408.1 | III | 2 |
| 710 | GCA_000350625.1 | 1169324 | 562 | Escherichia coli KTE2 | KTE2 | Proteobacteria | KB732209.1 | III | 2 |
| 711 | GCA_000408185.1 | 1181746 | 562 | Escherichia coli KTE200 | KTE200 | Proteobacteria | KE136942.1 | III | 2 |
| 712 | GCA_000352965.1 | 1181748 | 562 | Escherichia coli KTE202 | KTE202 | Proteobacteria | KB733186.1 | III | 2 |
| 713 | GCA_000351065.1 | 1181750 | 562 | Escherichia coli KTE204 | KTE204 | Proteobacteria | KB732469.1 | III | 2 |
| 714 | GCA_000351125.1 | 1181754 | 562 | Escherichia coli KTE208 | KTE208 | Proteobacteria | KB732511.1 | III | 2 |
| 715 | GCA_000408245.1 | 1181768 | 562 | Escherichia coli KTE222 | KTE222 | Proteobacteria | KE136972.1 | III | 2 |
| 716 | GCA_000408265.1 | 1181771 | 562 | Escherichia coli KTE225 | KTE225 | Proteobacteria | KE136980.1 | III | 2 |
| 717 | GCA_000351365.1 | 1181779 | 562 | Escherichia coli KTE235 | KTE235 | Proteobacteria | KB732610.1 | III | 2 |
| 718 | GCA_000350825.1 | 1169345 | 562 | Escherichia coli KTE26 | KTE26 | Proteobacteria | KB732334.1 | III | 2 |
| 719 | GCA_000350885.1 | 1169361 | 562 | Escherichia coli KTE44 | KTE44 | Proteobacteria | KB732364.1 | III | 2 |
| 720 | GCA_000352325.1 | 1182657 | 562 | Escherichia coli KTE50 | KTE50 | Proteobacteria | KB733431.1 | III | 2 |
| 721 | GCA_000351465.1 | 1182658 | 562 | Escherichia coli KTE51 | KTE51 | Proteobacteria | KB732658.1 | III | 2 |
| 722 | GCA_000351525.1 | 1182663 | 562 | Escherichia coli KTE56 | KTE56 | Proteobacteria | KB732702.1 | III | 2 |
| 723 | GCA_000408385.1 | 1182675 | 562 | Escherichia coli KTE68 | KTE68 | Proteobacteria | KE137040.1 | III | 2 |
| 724 | GCA_000351765.1 | 1182688 | 562 | Escherichia coli KTE81 | KTE81 | Proteobacteria | KB732854.1 | III | 2 |
| 725 | GCA_000326505.1 | 1169363 | 562 | Escherichia coli KTE82 | KTE82 | Proteobacteria | ANYT01000130.1 | III | 2 |
| 726 | GCA_000351785.1 | 1182689 | 562 | Escherichia coli KTE83 | KTE83 | Proteobacteria | KB733405.1 | III | 2 |
| 727 | GCA_000176575.2 | 656419 | 562 | Escherichia coli M718 | M718 | Proteobacteria | GL884102.1 | III | 2 |
| 728 | GCA_000261145.1 | 656421 | 562 | Escherichia coli M919 | M919 | Proteobacteria | JH659569.1 | III | 2 |
| 729 | GCA_000692775.1 | 1438688 | 562 | Escherichia coli MGH 58 | MGH 58 | Proteobacteria | KK736506.1 | III | 2 |
| 730 | GCA_000164275.1 | 749540 | 562 | Escherichia coli MS 146-1 | MS 146-1 | Proteobacteria | GG772126.1 | III | 2 |
| 731 | GCA_000164335.1 | 749547 | 562 | Escherichia coli MS 187-1 | MS 187-1 | Proteobacteria | GG772497.1 | III | 2 |
| 732 | GCA_000164555.1 | 749548 | 562 | Escherichia coli MS 196-1 | MS 196-1 | Proteobacteria | GG773987.1 | III | 2 |
| 733 | GCA_000164195.1 | 749549 | 562 | Escherichia coli MS 198-1 | MS 198-1 | Proteobacteria | GG771459.1 | III | 2 |
| 734 | GCA_000303835.2 | 1005484 | 562 | Escherichia coli NE037 | NE037 | Proteobacteria | JH960043.1 | III | 2 |
| 735 | GCA_000303295.2 | 1005486 | 562 | Escherichia coli NE098 | NE098 | Proteobacteria | JH946265.1 | III | 2 |
| 736 | GCA_000350045.2 | 1090929 | 562 | Escherichia coli O127:H27 str. C43/90 | C43/90 | Proteobacteria | AHAW01000071.1 | III | 2 |
| 737 | GCA_000695155.1 | 1078035 | 562 | Escherichia coli O145:H28 str. 4865/96 | 4865/96 | Proteobacteria | JHEY01000012.1 | III | 2 |
| 738 | GCA_000671295.1 | 1248823 | 562 | Escherichia coli O145:H28 str. RM12581 | RM12581 | Proteobacteria | CP007136.1 | III | 2 |
| 739 | GCA_000520035.1 | 1248902 | 562 | Escherichia coli O145:H28 str. RM13514 | RM13514 | Proteobacteria | CP006027.1 | III | 2 |
| 740 | GCA_000617345.1 | 1446585 | 562 | Escherichia coli O145:NM str. 06-3484 | 06-3484 | Proteobacteria | JHNN01000002.1 | III | 2 |
| 741 | GCA_000616665.2 | 1446586 | 562 | Escherichia coli O145:NM str. 08-4270 | 08-4270 | Proteobacteria | JHKV01000030.1 | III | 2 |
| 742 | GCA_000615085.1 | 1446587 | 562 | Escherichia coli O145:NM str. 2010C-3507 | 2010C-3507 | Proteobacteria | JHFW01000006.1 | III | 2 |
| 743 | GCA_000619185.1 | 1446588 | 562 | Escherichia coli O145:NM str. 2010C-3508 | 2010C-3508 | Proteobacteria | JHFV01000110.1 | III | 2 |
| 744 | GCA_000615175.1 | 1446590 | 562 | Escherichia coli O145:NM str. 2010C-3510 | 2010C-3510 | Proteobacteria | JHFT01000001.1 | III | 2 |
| 745 | GCA_000615225.2 | 1446591 | 562 | Escherichia coli O145:NM str. 2010C-3511 | 2010C-3511 | Proteobacteria | JHFS01000035.1 | III | 2 |
| 746 | GCA_000615265.1 | 1446592 | 562 | Escherichia coli O145:NM str. 2010C-3516 | 2010C-3516 | Proteobacteria | JHFR01000094.1 | III | 2 |
| 747 | GCA_000615305.1 | 1446593 | 562 | Escherichia coli O145:NM str. 2010C-3517 | 2010C-3517 | Proteobacteria | JHFQ01000117.1 | III | 2 |
| 748 | GCA_000615345.1 | 1446594 | 562 | Escherichia coli O145:NM str. 2010C-3518 | 2010C-3518 | Proteobacteria | JHFP01000105.1 | III | 2 |
| 749 | GCA_000615385.1 | 1446595 | 562 | Escherichia coli O145:NM str. 2010C-3521 | 2010C-3521 | Proteobacteria | JHF001000022.1 | III | 2 |
| 750 | GCA_000615415.2 | 1446596 | 562 | Escherichia coli O145:NM str. 2010C-3526 | 2010C-3526 | Proteobacteria | JHFN01000018.1 | III | 2 |
| 751 | GCA_000617765.2 | 1446597 | 562 | Escherichia coli O145:NM str. 2010C-4557C2 | 2010C-4557C2 | Proteobacteria | JHNA01000067.1 | III | 2 |
| 752 | GCA_000632555.1 | 1446606 | 562 | Escherichia coli O157: str. 2010EL-2044 | 2010EL-2044 | Proteobacteria | JHME01000103.1 | III | 2 |
| 753 | GCA_000632655.1 | 1446607 | 562 | Escherichia coli O157: str. 2010EL-2045 | 2010EL-2045 | Proteobacteria | JHMD01000001.1 | III | 2 |
| 754 | GCA_000187305.2 | 926027 | 562 | Escherichia coli O157:H- str. 493-89 | 493-89 | Proteobacteria | AETY01000104.1 | III | 2 |
| 755 | GCA_000187325.2 | 926028 | 562 | Escherichia coli O157:H- str. H 2687 | H 2687 | Proteobacteria | AETZ01000110.1 | III | 2 |
| 756 | GCA_000978815.1 | 83334 | 562 | Escherichia coli O157:H7 | 7.1_Anguil | Proteobacteria | LAZD01000130.1 | III | 2 |
| 757 | GCA_000978845.1 | 83334 | 562 | Escherichia coli O157:H7 | Rafaela_II | Proteobacteria | LAYW01000314.1 | III | 2 |
| 758 | GCA_001006425.1 | 83334 | 562 | Escherichia coli O157:H7 | HB74 | Proteobacteria | LCWU01000234.1 | III | 2 |
| 759 | GCA_001307215.1 | 83334 | 562 | Escherichia coli O157:H7 | WS4202 | Proteobacteria | CP012802.1 | III | 2 |
| 760 | GCA_001440045.1 | 83334 | 562 | Escherichia coli O157:H7 | G10 | Proteobacteria | LMXL01000097.1 | III | 2 |
| 761 | GCA_000617445.2 | 1446609 | 562 | Escherichia coli O157:H7 str. 06-3745 | 06-3745 | Proteobacteria | JHNI01000020.1 | III | 2 |
| 762 | GCA_000617705.2 | 1446610 | 562 | Escherichia coli O157:H7 str. 06-4039 | 06-4039 | Proteobacteria | JHNG01000005.1 | III | 2 |
| 763 | GCA_000617665.2 | 1446611 | 562 | Escherichia coli O157:H7 str. 07-3091 | 07-3091 | Proteobacteria | JHNF01000007.1 | III | 2 |
| 764 | GCA_000617725.2 | 1446612 | 562 | Escherichia coli O157:H7 str. 07-3391 | 07-3391 | Proteobacteria | JHNE01000001.1 | III | 2 |
| 765 | GCA_000616585.2 | 1446613 | 562 | Escherichia coli O157:H7 str. 08-3037 | 08-3037 | Proteobacteria | JHKZ01000011.1 | III | 2 |
| 766 | GCA_000616605.2 | 1446614 | 562 | Escherichia coli O157:H7 str. 08-3527 | 08-3527 | Proteobacteria | JHKY01000032.1 | III | 2 |
| 767 | GCA_000616645.2 | 1446615 | 562 | Escherichia coli O157:H7 str. 08-4169 | 08-4169 | Proteobacteria | JHKW01000004.1 | III | 2 |
| 768 | GCA_000619665.1 | 1446616 | 562 | Escherichia coli O157:H7 str. 08-4529 | 08-4529 | Proteobacteria | JHHI01000009.1 | III | 2 |
| 769 | GCA_000619145.1 | 1446618 | 562 | Escherichia coli O157:H7 str. 2009EL1449 | 2009EL1449 | Proteobacteria | JHGF01000005.1 | III | 2 |
| 770 | GCA_000614965.1 | 1446619 | 562 | Escherichia coli O157:H7 str. 2009EL1705 | 2009EL1705 | Proteobacteria | JHGE01000007.1 | III | 2 |
| 771 | GCA_000619165.1 | 1446620 | 562 | Escherichia coli O157:H7 str. 2009EL1913 | 2009EL1913 | Proteobacteria | JHGD01000001.1 | III | 2 |
| 772 | GCA_000615015.1 | 1446621 | 562 | Escherichia coli O157:H7 str. 2009EL2109 | 2009EL2109 | Proteobacteria | JHGC01000001.1 | III | 2 |
| 773 | GCA_000616365.1 | 1446623 | 562 | Escherichia coli O157:H7 str. 2011EL-1107 | 2011EL-1107 | Proteobacteria | JHLK01000001.1 | III | 2 |
| 774 | GCA_000616405.2 | 1446624 | 562 | Escherichia coli O157:H7 str. 2011EL-2090 | 2011EL-2090 | Proteobacteria | JHLI01000033.1 | III | 2 |
| 775 | GCA_000616425.2 | 1446625 | 562 | Escherichia coli O157:H7 str. 2011EL-2091 | 2011EL-2091 | Proteobacteria | JHLH01000026.1 | III | 2 |
| 776 | GCA_000616445.2 | 1446626 | 562 | Escherichia coli O157:H7 str. 2011EL-2092 | 2011EL-2092 | Proteobacteria | JHLG01000030.1 | III | 2 |
| 777 | GCA_000616465.2 | 1446627 | 562 | Escherichia coli O157:H7 str. 2011EL-2093 | 2011EL-2093 | Proteobacteria | JHLF01000012.1 | III | 2 |
| 778 | GCA_000616485.2 | 1446628 | 562 | Escherichia coli O157:H7 str. 2011EL-2094 | 2011EL-2094 | Proteobacteria | JHLE01000001.1 | III | 2 |
| 779 | GCA_000616505.2 | 1446629 | 562 | Escherichia coli O157:H7 str. 2011EL-2096 | 2011EL-2096 | Proteobacteria | JHLD01000033.1 | III | 2 |
| 780 | GCA_000616525.2 | 1446630 | 562 | Escherichia coli O157:H7 str. 2011EL-2097 | 2011EL-2097 | Proteobacteria | JHLC01000005.1 | III | 2 |
| 781 | GCA_000616545.2 | 1446631 | 562 | Escherichia coli O157:H7 str. 2011EL-2098 | 2011EL-2098 | Proteobacteria | JHLB01000003.1 | III | 2 |
| 782 | GCA_000616705.2 | 1446632 | 562 | Escherichia coli O157:H7 str. 2011EL-2099 | 2011EL-2099 | Proteobacteria | JHKT01000015.1 | III | 2 |
| 783 | GCA_000615695.1 | 1446633 | 562 | Escherichia coli O157:H7 str. 2011EL-2101 | 2011EL-2101 | Proteobacteria | JHKS01000001.1 | III | 2 |
| 784 | GCA_000615745.2 | 1446634 | 562 | Escherichia coli O157:H7 str. 2011EL-2103 | 2011EL-2103 | Proteobacteria | JHKR01000031.1 | III | 2 |
| 785 | GCA_000615785.2 | 1446635 | 562 | Escherichia coli O157:H7 str. 2011EL-2104 | 2011EL-2104 | Proteobacteria | JHKQ01000016.1 | III | 2 |
| 786 | GCA_000615815.2 | 1446636 | 562 | Escherichia coli O157:H7 str. 2011EL-2105 | 2011EL-2105 | Proteobacteria | JHKP01000014.1 | III | 2 |
| 787 | GCA_000615865.2 | 1446637 | 562 | Escherichia coli O157:H7 str. 2011EL-2106 | 2011EL-2106 | Proteobacteria | JHKO01000009.1 | III | 2 |
| 788 | GCA_000615925.2 | 1446638 | 562 | Escherichia coli O157:H7 str. 2011EL-2107 | 2011EL-2107 | Proteobacteria | JHKN01000006.1 | III | 2 |
| 789 | GCA_000615965.2 | 1446639 | 562 | Escherichia coli O157:H7 str. 2011EL-2108 | 2011EL-2108 | Proteobacteria | JHKM01000031.1 | III | 2 |
| 790 | GCA_000616005.2 | 1446640 | 562 | Escherichia coli O157:H7 str. 2011EL-2109 | 2011EL-2109 | Proteobacteria | JHKL01000015.1 | III | 2 |
| 791 | GCA_000616035.2 | 1446641 | 562 | Escherichia coli O157:H7 str. 2011EL-2111 | 2011EL-2111 | Proteobacteria | JHKK01000003.1 | III | 2 |
| 792 | GCA_000616075.2 | 1446642 | 562 | Escherichia coli O157:H7 str. 2011EL-2112 | 2011EL-2112 | Proteobacteria | JHKJ01000005.1 | III | 2 |
| 793 | GCA_000614645.2 | 1446643 | 562 | Escherichia coli O157:H7 str. 2011EL-2113 | 2011EL-2113 | Proteobacteria | JHKI01000015.1 | III | 2 |
| 794 | GCA_000614255.1 | 1446644 | 562 | Escherichia coli O157:H7 str. 2011EL-2114 | 2011EL-2114 | Proteobacteria | JHKH01000002.1 | III | 2 |
| 795 | GCA_000614305.2 | 1446645 | 562 | Escherichia coli O157:H7 str. 2011EL-2286 | 2011EL-2286 | Proteobacteria | JHKG01000013.1 | III | 2 |
| 796 | GCA_000614405.2 | 1446646 | 562 | Escherichia coli O157:H7 str. 2011EL-2287 | 2011EL-2287 | Proteobacteria | JHKF01000001.1 | III | 2 |
| 797 | GCA_000614085.2 | 1446647 | 562 | Escherichia coli O157:H7 str. 2011EL-2288 | 2011EL-2288 | Proteobacteria | JHKE01000007.1 | III | 2 |
| 798 | GCA_000614685.2 | 1446648 | 562 | Escherichia coli O157:H7 str. 2011EL-2289 | 2011EL-2289 | Proteobacteria | JHKD01000001.1 | III | 2 |
| 799 | GCA_000618945.2 | 1446649 | 562 | Escherichia coli O157:H7 str. 2011EL-2290 | 2011EL-2290 | Proteobacteria | JHKC01000008.1 | III | 2 |
| 800 | GCA_000614345.2 | 1446650 | 562 | Escherichia coli O157:H7 str. 2011EL-2312 | 2011EL-2312 | Proteobacteria | JHKB01000003.1 | III | 2 |
| 801 | GCA_000618985.2 | 1446651 | 562 | Escherichia coli O157:H7 str. 2011EL-2313 | 2011EL-2313 | Proteobacteria | JHKA01000028.1 | III | 2 |
| 802 | GCA_000181775.1 | 444449 | 562 | Escherichia coli O157:H7 str. EC4042 | EC4042 | Proteobacteria | ABHM02000001.1 | III | 2 |
| 803 | GCA_000181755.1 | 444448 | 562 | Escherichia coli O157:H7 str. EC4045 | EC4045 | Proteobacteria | ABHL02000001.1 | III | 2 |
| 804 | GCA_000171955.1 | 444453 | 562 | Escherichia coli O157:H7 str. EC4076 | EC4076 | Proteobacteria | ABHQ01000017.1 | III | 2 |
| 805 | GCA_000171935.1 | 444452 | 562 | Escherichia coli O157:H7 str. EC4113 | EC4113 | Proteobacteria | ABHP01000015.1 | III | 2 |
| 806 | GCA_000021125.1 | 444450 | 562 | Escherichia coli O157:H7 str. EC4115 | EC4115 | Proteobacteria | CP001164.1 | III | 2 |
| 807 | GCA_000171915.1 | 444451 | 562 | Escherichia coli O157:H7 str. EC4196 | EC4196 | Proteobacteria | ABHO01000006.1 | III | 2 |
| 808 | GCA_000181735.1 | 444447 | 562 | Escherichia coli O157:H7 str. EC4206 | EC4206 | Proteobacteria | ABHK02000001.1 | III | 2 |
| 809 | GCA_000171975.1 | 478004 | 562 | Escherichia coli O157:H7 str. EC4401 | EC4401 | Proteobacteria | ABHR01000005.1 | III | 2 |
| 810 | GCA_000171995.1 | 478005 | 562 | Escherichia coli O157:H7 str. EC4486 | EC4486 | Proteobacteria | ABHS01000002.1 | III | 2 |
| 811 | GCA_000172015.1 | 478006 | 562 | Escherichia coli O157:H7 str. EC4501 | EC4501 | Proteobacteria | ABHT01000004.1 | III | 2 |
| 812 | GCA_000172055.1 | 478007 | 562 | Escherichia coli O157:H7 str. EC508 | EC508 | Proteobacteria | ABHW01000003.1 | III | 2 |
| 813 | GCA_000006665.1 | 155864 | 562 | Escherichia coli O157:H7 str. EDL933 | EDL933 | Proteobacteria | AE005174.2 | III | 2 |
| 814 | GCA_000732965.1 | 155864 | 562 | Escherichia coli O157:H7 str. EDL933 | EDL933 | Proteobacteria | CP008957.1 | III | 2 |
| 815 | GCA_000618105.2 | 1446652 | 562 | Escherichia coli O157:H7 str. F6142 | F6142 | Proteobacteria | JHJT01000009.1 | III | 2 |
| 816 | GCA_000618045.1 | 1446653 | 562 | Escherichia coli O157:H7 str. F6749 | F6749 | Proteobacteria | JHJQ01000001.1 | III | 2 |
| 817 | GCA_000618305.2 | 1446654 | 562 | Escherichia coli O157:H7 str. F6750 | F6750 | Proteobacteria | JHJP01000010.1 | III | 2 |
| 818 | GCA_000618265.2 | 1446655 | 562 | Escherichia coli O157:H7 str. F6751 | F6751 | Proteobacteria | JHJO01000004.1 | III | 2 |
| 819 | GCA_000614805.2 | 1446656 | 562 | Escherichia coli O157:H7 str. F7350 | F7350 | Proteobacteria | JHJN01000036.1 | III | 2 |
| 820 | GCA_000618225.2 | 1446657 | 562 | Escherichia coli O157:H7 str. F7377 | F7377 | Proteobacteria | JHJM01000002.1 | III | 2 |
| 821 | GCA_000618065.2 | 1446658 | 562 | Escherichia coli O157:H7 str. F7384 | F7384 | Proteobacteria | JHJL01000002.1 | III | 2 |
| 822 | GCA_000618145.2 | 1446659 | 562 | Escherichia coli O157:H7 str. F7410 | F7410 | Proteobacteria | JHJK01000004.1 | III | 2 |
| 823 | GCA_000187285.4 | 926026 | 562 | Escherichia coli O157:H7 str. G5101 | G5101 | Proteobacteria | AETX01000114.1 | III | 2 |
| 824 | GCA_000618185.1 | 1446660 | 562 | Escherichia coli O157:H7 str. G5303 | G5303 | Proteobacteria | JHJI01000027.1 | III | 2 |
| 825 | GCA_000618245.1 | 1446661 | 562 | Escherichia coli O157:H7 str. H2495 | H2495 | Proteobacteria | JHJH01000020.1 | III | 2 |
| 826 | GCA_000618285.2 | 1446662 | 562 | Escherichia coli O157:H7 str. H2498 | H2498 | Proteobacteria | JHJG01000003.1 | III | 2 |
| 827 | GCA_000618025.1 | 1446663 | 562 | Escherichia coli O157:H7 str. K1420 | K1420 | Proteobacteria | JHJF01000008.1 | III | 2 |
| 828 | GCA_000618425.1 | 1446664 | 562 | Escherichia coli O157:H7 str. K1792 | K1792 | Proteobacteria | JHJD01000136.1 | III | 2 |
| 829 | GCA_000618365.1 | 1446665 | 562 | Escherichia coli O157:H7 str. K1793 | K1793 | Proteobacteria | JHJC01000041.1 | III | 2 |
| 830 | GCA_000618325.1 | 1446667 | 562 | Escherichia coli O157:H7 str. K1796 | K1796 | Proteobacteria | JHJA01000003.1 | III | 2 |
| 831 | GCA_000618345.1 | 1446668 | 562 | Escherichia coli O157:H7 str. K1845 | K1845 | Proteobacteria | JHIZ01000038.1 | III | 2 |
| 832 | GCA_000618445.1 | 1446669 | 562 | Escherichia coli O157:H7 str. K1921 | K1921 | Proteobacteria | JHIY01000012.1 | III | 2 |
| 833 | GCA_000618465.1 | 1446670 | 562 | Escherichia coli O157:H7 str. K1927 | K1927 | Proteobacteria | JHIX01000108.1 | III | 2 |
| 834 | GCA_000618485.1 | 1446671 | 562 | Escherichia coli O157:H7 str. K2188 | K2188 | Proteobacteria | JHIW01000103.1 | III | 2 |
| 835 | GCA_000618665.1 | 1446674 | 562 | Escherichia coli O157:H7 str. K2324 | K2324 | Proteobacteria | JHIT01000105.1 | III | 2 |
| 836 | GCA_000618525.1 | 1446675 | 562 | Escherichia coli O157:H7 str. K2581 | K2581 | Proteobacteria | JHIS01000002.1 | III | 2 |
| 837 | GCA_000618605.1 | 1446679 | 562 | Escherichia coli O157:H7 str. K4396 | K4396 | Proteobacteria | JHI001000002.1 | III | 2 |
| 838 | GCA_000617965.1 | 1446680 | 562 | Escherichia coli O157:H7 str. K4405 | K4405 | Proteobacteria | JHIN01000100.1 | III | 2 |
| 839 | GCA_000618625.1 | 1446681 | 562 | Escherichia coli O157:H7 str. K4406 | K4406 | Proteobacteria | JHIM01000001.1 | III | 2 |
| 840 | GCA_000618685.1 | 1446682 | 562 | Escherichia coli O157:H7 str. K4527 | K4527 | Proteobacteria | JHIL01000001.1 | III | 2 |
| 841 | GCA_000618745.1 | 1446683 | 562 | Escherichia coli O157:H7 str. K5418 | K5418 | Proteobacteria | JHII01000001.1 | III | 2 |
| 842 | GCA_000618765.1 | 1446684 | 562 | Escherichia coli O157:H7 str. K5448 | K5448 | Proteobacteria | JHIH01000007.1 | III | 2 |
| 843 | GCA_000618785.1 | 1446685 | 562 | Escherichia coli O157:H7 str. K5449 | K5449 | Proteobacteria | JHIG01000002.1 | III | 2 |
| 844 | GCA_000618805.1 | 1446686 | 562 | Escherichia coli O157:H7 str. K5453 | K5453 | Proteobacteria | JHIF01000066.1 | III | 2 |
| 845 | GCA_000618885.1 | 1446687 | 562 | Escherichia coli O157:H7 str. K5460 | K5460 | Proteobacteria | JHIE01000002.1 | III | 2 |
| 846 | GCA_000618825.1 | 1446688 | 562 | Escherichia coli O157:H7 str. K5467 | K5467 | Proteobacteria | JHID01000001.1 | III | 2 |
| 847 | GCA_000618845.1 | 1446689 | 562 | Escherichia coli O157:H7 str. K5602 | K5602 | Proteobacteria | JHIC01000015.1 | III | 2 |
| 848 | GCA_000618865.1 | 1446690 | 562 | Escherichia coli O157:H7 str. K5607 | K5607 | Proteobacteria | JHIB01000088.1 | III | 2 |
| 849 | GCA_000619445.1 | 1446691 | 562 | Escherichia coli O157:H7 str. K5609 | K5609 | Proteobacteria | JHIA01000021.1 | III | 2 |
| 850 | GCA_000616745.1 | 1446692 | 562 | Escherichia coli O157:H7 str. K5806 | K5806 | Proteobacteria | JHHZ01000001.1 | III | 2 |
| 851 | GCA_000619365.1 | 1446693 | 562 | Escherichia coli O157:H7 str. K5852 | K5852 | Proteobacteria | JHHY01000003.1 | III | 2 |
| 852 | GCA_000619385.1 | 1446694 | 562 | Escherichia coli O157:H7 str. K6590 | K6590 | Proteobacteria | JHHX01000004.1 | III | 2 |
| 853 | GCA_000619405.1 | 1446695 | 562 | Escherichia coli O157:H7 str. K6676 | K6676 | Proteobacteria | JHHW01000001.1 | III | 2 |
| 854 | GCA_000619425.1 | 1446696 | 562 | Escherichia coli O157:H7 str. K6687 | K6687 | Proteobacteria | JHHV01000121.1 | III | 2 |
| 855 | GCA_000618925.2 | 1446697 | 562 | Escherichia coli O157:H7 str. k7140 | K7140 | Proteobacteria | JHHK01000016.1 | III | 2 |
| 856 | GCA_000008865.1 | 386585 | 562 | Escherichia coli O157:H7 str. Sakai | Sakai substr. RIMD | Proteobacteria | BA000007.2 | III | 2 |
| | | | | | 0509952 | | | | |
| 857 | GCA_000730345.1 | 1328859 | 562 | Escherichia coli O157:H7 str. SS17 | SS17 | Proteobacteria | CP008805.1 | III | 2 |
| 858 | GCA_000803705.1 | 1330457 | 562 | Escherichia coli O157:H7 str. SS52 | SS52 | Proteobacteria | CP010304.1 | III | 2 |
| 859 | GCA_000022225.1 | 544404 | 562 | Escherichia coli O157:H7 str. TW14359 | TW14359 | Proteobacteria | CP001368.1 | III | 2 |
| 860 | GCA_000155125.1 | 502346 | 562 | Escherichia coli O157:H7 str. TW14588 | TW14588 | Proteobacteria | CM000662.1 | III | 2 |
| 861 | GCA_000617385.1 | 1446745 | 562 | Escherichia coli O55:H7 str. 06-3555 | 06-3555 | Proteobacteria | JHNL01000009.1 | III | 2 |
| 862 | GCA_000187345.2 | 926029 | 562 | Escherichia coli O55:H7 str. 3256-97 | 3256-97 TW 07815 | Proteobacteria | AEUA01000088.1 | III | 2 |
| 863 | GCA_000025165.1 | 701177 | 562 | Escherichia coli O55:H7 str. CB9615 | CB9615 | Proteobacteria | CP001846.1 | III | 2 |
| 864 | GCA_000357145.2 | 1116131 | 562 | Escherichia coli P02997067.6 | P02997067.6 | Proteobacteria | APYJ01000201.1 | III | 2 |
| 865 | GCA_000356665.2 | 1116047 | 562 | Escherichia coli P0299917.1 | P0299917.1 | Proteobacteria | AQER01000001.1 | III | 2 |
| 866 | GCA_000356605.2 | 1116089 | 562 | Escherichia coli p0305293.1 | p0305293.1 | Proteobacteria | AQEK01000045.1 | III | 2 |
| 867 | GCA_000358555.1 | 1116098 | 562 | Escherichia coli p0305293.10 | p0305293.10 | Proteobacteria | AQBT01000187.1 | III | 2 |
| 868 | GCA_000358575.2 | 1116099 | 562 | Escherichia coli p0305293.11 | p0305293.11 | Proteobacteria | AQBU01000292.1 | III | 2 |
| 869 | GCA_000358595.2 | 1116100 | 562 | Escherichia coli p0305293.12 | p0305293.12 | Proteobacteria | AQBV01000163.1 | III | 2 |
| 870 | GCA_000356865.2 | 1116101 | 562 | Escherichia coli p0305293.13 | p0305293.13 | Proteobacteria | APZI01000451.1 | III | 2 |
| 871 | GCA_000357485.2 | 1125646 | 562 | Escherichia coli p0305293.14 | p0305293.14 | Proteobacteria | APZZ01000159.1 | III | 2 |
| 872 | GCA_000358615.2 | 1116102 | 562 | Escherichia coli p0305293.15 | p0305293.15 | Proteobacteria | AQBW01000104.1 | III | 2 |
| 873 | GCA_000358635.2 | 1116090 | 562 | Escherichia coli p0305293.2 | p0305293.2 | Proteobacteria | AQBX01000150.1 | III | 2 |
| 874 | GCA_000358655.2 | 1116091 | 562 | Escherichia coli p0305293.3 | p0305293.3 | Proteobacteria | AQBY01000159.1 | III | 2 |
| 875 | GCA_000358675.2 | 1116092 | 562 | Escherichia coli p0305293.4 | p0305293.4 | Proteobacteria | AQBZ01000242.1 | III | 2 |
| 876 | GCA_000359055.2 | 1116093 | 562 | Escherichia coli p0305293.5 | p0305293.5 | Proteobacteria | AQCA01000251.1 | III | 2 |
| 877 | GCA_000358695.2 | 1116096 | 562 | Escherichia coli p0305293.8 | p0305293.8 | Proteobacteria | AQCD01000162.1 | III | 2 |
| 878 | GCA_000358715.2 | 1116097 | 562 | Escherichia coli p0305293.9 | p0305293.9 | Proteobacteria | AQCE01000149.1 | III | 2 |
| 879 | GCA_000498255.1 | 1097579 | 562 | Escherichia coli P4-96 | P4-96 | Proteobacteria | AHH001000034.1 | III | 2 |
| 880 | GCA_000498275.1 | 1117111 | 562 | Escherichia coli P4-NR | P4-NR | Proteobacteria | AHHP01000046.1 | III | 2 |
| 881 | GCA_000267865.2 | 1005494 | 562 | Escherichia coli PA10 | PA10 | Proteobacteria | JH709066.1 | III | 2 |
| 882 | GCA_000335415.2 | 1051349 | 562 | Escherichia coli PA11 | PA11 | Proteobacteria | AOEI01000002.1 | III | 2 |
| 883 | GCA_000335435.2 | 1051350 | 562 | Escherichia coli PA13 | PA13 | Proteobacteria | AOEK01000001.1 | III | 2 |
| 884 | GCA_000267845.2 | 1005495 | 562 | Escherichia coli PA14 | PA14 | Proteobacteria | JH708600.1 | III | 2 |
| 885 | GCA_000267825.2 | 1005496 | 562 | Escherichia coli PA15 | PA15 | Proteobacteria | JH707559.1 | III | 2 |
| 886 | GCA_000335335.2 | 1051351 | 562 | Escherichia coli PA19 | PA19 | Proteobacteria | AOEJ01000133.1 | III | 2 |
| 887 | GCA_000335355.2 | 1005487 | 562 | Escherichia coli PA2 | PA2 | Proteobacteria | AOEL01000002.1 | III | 2 |
| 888 | GCA_000267085.1 | 1005497 | 562 | Escherichia coli PA22 | PA22 | Proteobacteria | AKLI01000001.1 | III | 2 |
| 889 | GCA_000303895.2 | 1005498 | 562 | Escherichia coli PA23 | PA23 | Proteobacteria | JH959990.1 | III | 2 |
| 890 | GCA_000267805.2 | 1005499 | 562 | Escherichia coli PA24 | PA24 | Proteobacteria | JH707528.1 | III | 2 |
| 891 | GCA_000267785.2 | 1005500 | 562 | Escherichia coli PA25 | PA25 | Proteobacteria | JH706538.1 | III | 2 |
| 892 | GCA_000267765.2 | 1005501 | 562 | Escherichia coli PA28 | PA28 | Proteobacteria | JH705685.1 | III | 2 |
| 893 | GCA_000267925.2 | 1005488 | 562 | Escherichia coli PA3 | PA3 | Proteobacteria | JH711390.1 | III | 2 |
| 894 | GCA_000267745.2 | 1005502 | 562 | Escherichia coli PA31 | PA31 | Proteobacteria | JH707077.1 | III | 2 |
| 895 | GCA_000267725.2 | 1005503 | 562 | Escherichia coli PA32 | PA32 | Proteobacteria | JH705678.1 | III | 2 |
| 896 | GCA_000267705.2 | 1005504 | 562 | Escherichia coli PA33 | PA33 | Proteobacteria | JH705112.1 | III | 2 |
| 897 | GCA_000303695.2 | 1005505 | 562 | Escherichia coli PA34 | PA34 | Proteobacteria | JH963946.1 | III | 2 |
| 898 | GCA_000335175.2 | 1051352 | 562 | Escherichia coli PA35 | PA35 | Proteobacteria | AOES01000151.1 | III | 2 |
| 899 | GCA_000303675.2 | 1005506 | 562 | Escherichia coli PA38 | PA38 | Proteobacteria | JH966118.1 | III | 2 |
| 900 | GCA_000267685.2 | 1005507 | 562 | Escherichia coli PA39 | PA39 | Proteobacteria | JH704564.1 | III | 2 |
| 901 | GCA_000303875.2 | 1005489 | 562 | Escherichia coli PA4 | PA4 | Proteobacteria | JH960549.1 | III | 2 |
| 902 | GCA_000267645.2 | 1005510 | 562 | Escherichia coli PA42 | PA42 | Proteobacteria | JH702836.1 | III | 2 |
| 903 | GCA_000303935.2 | 1005511 | 562 | Escherichia coli PA45 | PA45 | Proteobacteria | JH958691.1 | III | 2 |
| 904 | GCA_000335075.2 | 1005512 | 562 | Escherichia coli PA47 | PA47 | Proteobacteria | AOEM01000157.1 | III | 2 |
| 905 | GCA_000335095.2 | 1005513 | 562 | Escherichia coli PA48 | PA48 | Proteobacteria | AOEN01000122.1 | III | 2 |
| 906 | GCA_000303915.2 | 1005514 | 562 | Escherichia coli PA49 | PA49 | Proteobacteria | JH959133.1 | III | 2 |
| 907 | GCA_000267905.2 | 1005490 | 562 | Escherichia coli PA5 | PA5 | Proteobacteria | JH709975.1 | III | 2 |
| 908 | GCA_000302735.1 | 1005491 | 562 | Escherichia coli PA7 | PA7 | Proteobacteria | AMTQ01000008.1 | III | 2 |
| 909 | GCA_000335455.2 | 1005492 | 562 | Escherichia coli PA8 | PA8 | Proteobacteria | AOEO01000008.1 | III | 2 |
| 910 | GCA_000267885.2 | 1005493 | 562 | Escherichia coli PA9 | PA9 | Proteobacteria | JH709102.1 | III | 2 |
| 911 | GCA_000019645.1 | 439855 | 562 | Escherichia coli SMS-3-5 | SMS-3-5 | Proteobacteria | CP000970.1 | III | 2 |
| 912 | GCA_000462145.2 | 1081891 | 562 | Escherichia coli T1282_01 | T1282_01 | Proteobacteria | AVRM01000132.1 | III | 2 |
| 913 | GCA_000462265.2 | 1081896 | 562 | Escherichia coli T1840_97 | T1840_97 | Proteobacteria | AVRN01000121.1 | III | 2 |
| 914 | GCA_000462385.2 | 1081892 | 562 | Escherichia coli T234_00 | T234_00 | Proteobacteria | AVRO01000124.1 | III | 2 |
| 915 | GCA_000462165.2 | 1081893 | 562 | Escherichia coli T924_01 | T924_01 | Proteobacteria | AVRP01000002.1 | III | 2 |
| 916 | GCA_000176615.2 | 656437 | 562 | Escherichia coli TA143 | TA143 | Proteobacteria | GL884236.1 | III | 2 |
| 917 | GCA_000303955.2 | 1005555 | 562 | Escherichia coli TT12B | TT12B | Proteobacteria | JH957216.1 | III | 2 |
| 918 | GCA_000267125.1 | 1005515 | 562 | Escherichia coli TW06591 | TW06591 | Proteobacteria | AKLT01000044.1 | III | 2 |
| 919 | GCA_000267625.2 | 1005516 | 562 | Escherichia coli TW07945 | TW07945 | Proteobacteria | JH702467.1 | III | 2 |
| 920 | GCA_000267605.2 | 1005519 | 562 | Escherichia coli TW09098 | TW09098 | Proteobacteria | JH702452.1 | III | 2 |
| 921 | GCA_000267065.1 | 1005520 | 562 | Escherichia coli TW09109 | TW09109 | Proteobacteria | AKLY01000005.1 | III | 2 |
| 922 | GCA_000267145.1 | 1005517 | 562 | Escherichia coli TW10246 | TW10246 | Proteobacteria | AKLV01000003.1 | III | 2 |
| 923 | GCA_000267165.1 | 1005518 | 562 | Escherichia coli TW11039 | TW11039 | Proteobacteria | AKLW01000006.1 | III | 2 |
| 924 | GCA_000267405.2 | 1005526 | 562 | Escherichia coli TW14301 | TW14301 | Proteobacteria | JH695863.1 | III | 2 |
| 925 | GCA_000267425.2 | 1005525 | 562 | Escherichia coli TW14313 | TW14313 | Proteobacteria | JH695401.1 | III | 2 |
| 926 | GCA_000462885.2 | 1207037 | 562 | Escherichia coli Tx1686 | Tx1686 | Proteobacteria | AVSN01000001.1 | III | 2 |
| 927 | GCA_000462905.2 | 1207036 | 562 | Escherichia coli Tx3800 | Tx3800 | Proteobacteria | AVS001000014.1 | III | 2 |
| 928 | GCA_000692835.1 | 1438693 | 562 | Escherichia coli UCI 57 | UCI 57 | Proteobacteria | KK736542.1 | III | 2 |
| 929 | GCA_000692855.1 | 1438694 | 562 | Escherichia coli UCI 58 | UCI 58 | Proteobacteria | KK736552.1 | III | 2 |
| 930 | GCA_000460315.1 | 1281191 | 562 | Escherichia coli UMEA 3144-1 | UMEA 3144-1 | Proteobacteria | KE701670.1 | III | 2 |
| 931 | GCA_000460355.1 | 1281194 | 562 | Escherichia coli UMEA 3151-1 | UMEA 3151-1 | Proteobacteria | KE701689.1 | III | 2 |
| 932 | GCA_000460755.1 | 1281214 | 562 | Escherichia coli UMEA 3201-1 | UMEA 3201-1 | Proteobacteria | KE701998.1 | III | 2 |
| 933 | GCA_000461015.1 | 1281227 | 562 | Escherichia coli UMEA 3240-1 | UMEA 3240-1 | Proteobacteria | KE702179.1 | III | 2 |
| 934 | GCA_000461115.1 | 1281233 | 562 | Escherichia coli UMEA 3271-1 | UMEA 3271-1 | Proteobacteria | KE702260.1 | III | 2 |
| 935 | GCA_000461255.1 | 1281242 | 562 | Escherichia coli UMEA 3329-1 | UMEA 3329-1 | Proteobacteria | KE702361.1 | III | 2 |
| 936 | GCA_000461315.1 | 1281247 | 562 | Escherichia coli UMEA 3355-1 | UMEA 3355-1 | Proteobacteria | KE702386.1 | III | 2 |
| 937 | GCA_000461675.1 | 1281269 | 562 | Escherichia coli UMEA 3718-1 | UMEA 3718-1 | Proteobacteria | KE702585.1 | III | 2 |
| 938 | GCA_000461855.1 | 1281278 | 562 | Escherichia coli UMEA 4076-1 | UMEA 4076-1 | Proteobacteria | KE702725.1 | III | 2 |
| 939 | GCA_000026325.1 | 585056 | 562 | Escherichia coli UMN026 | UMN026 | Proteobacteria | CU928163.2 | III | 2 |
| 940 | GCA_000262125.1 | 741093 | 562 | Escherichia coli Xuzhou21 | Xuzhou21 | Proteobacteria | CP001925.1 | III | 2 |
| 941 | GCA_000968905.2 | 564 | 564 | Escherichia fergusonii | GTA-EF02 | Proteobacteria | JZWQ02000016.1 | III | 2 |
| 942 | GCA_000026225.1 | 585054 | 564 | Escherichia fergusonii ATCC 35469 | | Proteobacteria | CU928158.2 | III | 2 |
| 943 | GCA_000190495.1 | 550694 | 564 | Escherichia fergusonii B253 | B253 | Proteobacteria | GL870856.1 | III | 2 |
| 944 | GCA_000408845.1 | 1279005 | 1279005 | Escherichia sp. KTE172 | KTE172 | Proteobacteria | KE137316.1 | III | 2 |
| 945 | GCA_000961035.1 | 35554 | 35554 | Geobacter sulfurreducens | AM-1 | Proteobacteria | CP010430.1 | III | 2 |
| 946 | GCA_000181475.1 | 344747 | 122 | Gimesia maris DSM 8797 | DSM 8797 | Planctomycetes | ABCE01000001.1 | III | 2 |
| 947 | GCA_000333895.2 | 1056512 | 1056512 | Grimontia indica | AK16 | Proteobacteria | ANFM02000056.1 | III | 2 |
| 948 | GCA_000961635.1 | 1629712 | 1629712 | Hoeflea sp. BRH_c9 | | Proteobacteria | LADQ01000154.1 | III | 2 |
| 949 | GCA_000152885.1 | 314276 | 190892 | Idiomarina baltica OS145 | OS145 | Proteobacteria | CH672405.1 | III | 2 |
| 950 | GCA_001577655.1 | 1794809 | 1794809 | Idiomarina sp. T82-3 | | Proteobacteria | LSBQ01000017.1 | III | 2 |
| 951 | GCA_000723165.1 | 1349767 | 55508 | Janthinobacterium agaricidamnosum NBRC 102515 = DSM 9628 | DSM 9628 | Proteobacteria | HG322949.1 | III | 2 |
| 952 | GCA_001038305.1 | 571 | 571 | Klebsiella oxytoca | GY84G39 | Proteobacteria | LDZM01000006.1 | III | 2 |
| 953 | GCA_001078255.1 | 883124 | 571 | Klebsiella oxytoca 10-5249 | 10-5249 | Proteobacteria | KQ235791.1 | III | 2 |
| 954 | GCA_000607265.1 | 1409787 | 571 | Klebsiella oxytoca G54 | G54 | Proteobacteria | KK097710.1 | III | 2 |
| 955 | GCA_000764635.1 | 573 | 573 | Klebsiella pneumoniae | BAMC 07-18 | Proteobacteria | JRQE01000009.1 | III | 2 |
| 956 | GCA_000786375.1 | 573 | 573 | Klebsiella pneumoniae | 223/14 | Proteobacteria | JRTV01000011.1 | III | 2 |
| 957 | GCA_000788005.1 | 573 | 573 | Klebsiella pneumoniae | 101712 | Proteobacteria | JSZJ01000008.1 | III | 2 |
| 958 | GCA_000807515.2 | 573 | 573 | Klebsiella pneumoniae | 6234 | Proteobacteria | JWRK01000001.1 | III | 2 |
| 959 | GCA_001031325.1 | 573 | 573 | Klebsiella pneumoniae | CHS112 | Proteobacteria | KQ088280.1 | III | 2 |
| 960 | GCA_001031795.1 | 573 | 573 | Klebsiella pneumoniae | CHS140 | Proteobacteria | KQ088469.1 | III | 2 |
| 961 | GCA_001033505.1 | 573 | 573 | Klebsiella pneumoniae | MGH84 | Proteobacteria | KQ089360.1 | III | 2 |
| 962 | GCA_001033525.1 | 573 | 573 | Klebsiella pneumoniae | MGH89 | Proteobacteria | KQ089366.1 | III | 2 |
| 963 | GCA_001033725.1 | 573 | 573 | Klebsiella pneumoniae | MGH101 | Proteobacteria | KQ089465.1 | III | 2 |
| 964 | GCA_001033925.1 | 573 | 573 | Klebsiella pneumoniae | MGH124 | Proteobacteria | KQ089571.1 | III | 2 |
| 965 | GCA_001185865.1 | 573 | 573 | Klebsiella pneumoniae | CHS175 | Proteobacteria | KQ236856.1 | III | 2 |
| 966 | GCA_001307175.1 | 573 | 573 | Klebsiella pneumoniae | KP617 | Proteobacteria | CP012753.1 | III | 2 |
| 967 | GCA_001441285.1 | 573 | 573 | Klebsiella pneumoniae | B86 | Proteobacteria | UCD01000021.1 | III | 2 |
| 968 | GCA_001456055.1 | 573 | 573 | Klebsiella pneumoniae | KpN01 | Proteobacteria | CP012987.1 | III | 2 |
| 969 | GCA_001456095.1 | 573 | 573 | Klebsiella pneumoniae | KpN06 | Proteobacteria | CP012992.1 | III | 2 |
| 970 | GCA_001462825.1 | 573 | 573 | Klebsiella pneumoniae | K68-18 | Proteobacteria | LNHE01000086.1 | III | 2 |
| 971 | GCA_001462845.1 | 573 | 573 | Klebsiella pneumoniae | K68-73 | Proteobacteria | LNHF01000070.1 | III | 2 |
| 972 | GCA_001463085.1 | 573 | 573 | Klebsiella pneumoniae | 50580255 | Proteobacteria | LNHK01000055.1 | III | 2 |
| 973 | GCA_001463165.1 | 573 | 573 | Klebsiella pneumoniae | U25 | Proteobacteria | CP012043.1 | III | 2 |
| 974 | GCA_001463475.1 | 573 | 573 | Klebsiella pneumoniae | 50884375 | Proteobacteria | LNIS01000067.1 | III | 2 |
| 975 | GCA_001483055.1 | 573 | 573 | Klebsiella pneumoniae | K1 | Proteobacteria | LOEJ01000055.1 | III | 2 |
| 976 | GCA_001483725.1 | 573 | 573 | Klebsiella pneumoniae | K45-67 | Proteobacteria | LLWB01000171.1 | III | 2 |
| 977 | GCA_001574505.1 | 573 | 573 | Klebsiella pneumoniae | U41 | Proteobacteria | LSTN01000011.1 | III | 2 |
| 978 | GCA_001597015.1 | 573 | 573 | Klebsiella pneumoniae | KP-33P | Proteobacteria | JXJG01000034.1 | III | 2 |
| 979 | GCA_000474635.1 | 1381121 | 573 | Klebsiella pneumoniae 303K | 303K | Proteobacteria | AVAN01000039.1 | III | 2 |
| 980 | GCA_000567405.1 | 1328405 | 573 | Klebsiella pneumoniae BIDMC 11 | BIDMC 11 | Proteobacteria | KK036859.1 | III | 2 |
| 981 | GCA_000492295.1 | 1328425 | 573 | Klebsiella pneumoniae BIDMC 36 | BIDMC 36 | Proteobacteria | KI535412.1 | III | 2 |
| 982 | GCA_000694235.1 | 1438772 | 573 | Klebsiella pneumoniae CHS 66 | CHS 66 | Proteobacteria | KK737378.1 | III | 2 |
| 983 | GCA_000530055.1 | 1432547 | 573 | Klebsiella pneumoniae IS22 | IS22 | Proteobacteria | CBWF010000048.1 | III | 2 |
| 984 | GCA_000316265.2 | 1236101 | 573 | Klebsiella pneumoniae JHCK1 | JHCK1 | Proteobacteria | ANGH02000162.1 | III | 2 |
| 985 | GCA_000406505.1 | 1284834 | 573 | Klebsiella pneumoniae KP-11 | KP-11 | Proteobacteria | APWE01000228.1 | III | 2 |
| 986 | GCA_000406385.1 | 1284833 | 573 | Klebsiella pneumoniae KP-7 | KP-7 | Proteobacteria | AQQD01000021.1 | III | 2 |
| 987 | GCA_000493135.1 | 1328363 | 573 | Klebsiella pneumoniae MGH 18 | MGH 18 | Proteobacteria | KI535680.1 | III | 2 |
| 988 | GCA_000567745.1 | 1328371 | 573 | Klebsiella pneumoniae MGH 35 | MGH 35 | Proteobacteria | KK036965.1 | III | 2 |
| 989 | GCA_000492775.1 | 1328378 | 573 | Klebsiella pneumoniae MGH 46 | MGH 46 | Proteobacteria | KI535587.1 | III | 2 |
| 990 | GCA_000694715.1 | 1438797 | 573 | Klebsiella pneumoniae MGH 74 | MGH 74 | Proteobacteria | KK737643.1 | III | 2 |
| 991 | GCA_000785005.1 | 72407 | 573 | Klebsiella pneumoniae subsp. pneumoniae | KPNIH31 | Proteobacteria | CP009876.1 | III | 2 |
| 992 | GCA_000733255.1 | 1406314 | 573 | Klebsiella pneumoniae subsp. pneumoniae PittNDM01 | PittNDM01 | Proteobacteria | CP006798.1 | III | 2 |
| 993 | GCA_000300675.1 | 1203545 | 573 | Klebsiella pneumoniae subsp. pneumoniae WGLW2 | WGLW2 | Proteobacteria | JH930419.1 | III | 2 |
| 994 | GCA_000492595.1 | 1328391 | 573 | Klebsiella pneumoniae UCICRE 4 | UCICRE 4 | Proteobacteria | KI535510.1 | III | 2 |
| 995 | GCA_000417465.1 | 1284818 | 573 | Klebsiella pneumoniae UHKPC179 | UHKPC179 | Proteobacteria | ARSM01000005.1 | III | 2 |
| 996 | GCA_000409125.1 | 1284817 | 573 | Klebsiella pneumoniae UHKPC57 | UHKPC57 | Proteobacteria | ARPR01000126.1 | III | 2 |
| 997 | GCA_000280495.1 | 1177180 | 283686 | Kosakonia radicincitans DSM 16656 | DSM 16656 | Proteobacteria | JH725436.1 | III | 2 |
| 998 | GCA_000691205.1 | 1455607 | 283686 | Kosakonia radicincitans UMEnt01/12 | UMEnt01/12 | Proteobacteria | JDYJ01000004.1 | III | 2 |
| 999 | GCA_000048665.1 | 297245 | 446 | Legionella pneumophila str. Lens | Lens | Proteobacteria | CR628337.1 | III | 2 |
| 1000 | GCA_001468025.1 | 1122169 | 45075 | Legionella shakespearei DSM 23087 | ATCC 49655 | Proteobacteria | LNYW01000016.1 | III | 2 |
| 1001 | GCA_000521805.1 | 1122207 | 263818 | Marinomonas ushuaiensis DSM 15871 | DSM 15871 | Proteobacteria | JAMB01000003.1 | III | 2 |
| 1002 | GCA_000502395.1 | 1287232 | 1287232 | Mesorhizobium sp. LNHC220B00 | LNHC220B00 | Proteobacteria | AYWS01000016.1 | III | 2 |
| 1003 | GCA_000502435.1 | 1287243 | 1287243 | Mesorhizobium sp. LNHC232B00 | LNHC232B00 | Proteobacteria | AYWP01000014.1 | III | 2 |
| 1004 | GCA_000502975.1 | 1287268 | 1287268 | Mesorhizobium sp. LNJC384A00 | LNJC384A00 | Proteobacteria | AYWK01000002.1 | III | 2 |
| 1005 | GCA_001425625.1 | 1736422 | 1736422 | Mesorhizobium sp. Root102 | Root102 | Proteobacteria | LMCP01000044.1 | III | 2 |
| 1006 | GCA_000364445.1 | 908290 | 39956 | Methylobacterium mesophilicum SR1.6/6 | SR1.6/6 | Proteobacteria | ANPA01000017.1 | III | 2 |
| 1007 | GCA_000364445.1 | 908290 | 39956 | Methylobacterium mesophilicum SR1.6/6 | SR1.6/6 | Proteobacteria | ANPA01000017.1 | III | 2 |
| 1008 | GCA_001423385.1 | 1736262 | 1736262 | Methylobacterium sp. Leaf121 | Leaf121 | Proteobacteria | LMNP01000003.1 | III | 2 |
| 1009 | GCA_001422165.1 | 1736242 | 1736242 | Methylobacterium sp. Leaf86 | Leaf86 | Proteobacteria | LMMG01000005.1 | III | 2 |
| 1010 | GCA_001422265.1 | 1736246 | 1736246 | Methylobacterium sp. Leaf90 | Leaf90 | Proteobacteria | LMMN01000010.1 | III | 2 |
| 1011 | GCA_000186165.1 | 435832 | 488 | Neisseria mucosa C102 | C102 | Proteobacteria | GL635793.1 | III | 2 |
| 1012 | GCA_000235975.2 | 1088721 | 205844 | Novosphingobium pentaromativorans US6-1 | US6-1 | Proteobacteria | AGFM01000037.1 | III | 2 |
| 1013 | GCA_000767465.1 | 1088721 | 205844 | Novosphingobium pentaromativorans US6-1 | US6-1 | Proteobacteria | CP009291.1 | III | 2 |
| 1014 | GCA_000253255.1 | 702113 | 702113 | Novosphingobium sp. PP1Y | PP1Y | Proteobacteria | FR856862.1 | III | 2 |
| 1015 | GCA_001476715.1 | 59814 | 59814 | Pantoea dispersa | NS215 | Proteobacteria | LDRY01000017.1 | III | 2 |
| 1016 | GCA_001477165.1 | 59814 | 59814 | Pantoea dispersa | RSA31 | Proteobacteria | LDSC01000016.1 | III | 2 |
| 1017 | GCA_000179655.1 | 517433 | 517433 | Pantoea sp. aB | aB | Proteobacteria | AEDL01000002.1 | III | 2 |
| 1018 | GCA_000754715.1 | 78398 | 554 | Pectobacterium carotovorum subsp. odoriferum | NCPPB3841 | Proteobacteria | JQOF01000009.1 | III | 2 |
| 1019 | GCA_000024645.1 | 561231 | 55208 | Pectobacterium wasabiae WPP163 | WPP163 | Proteobacteria | CP001790.1 | III | 2 |
| 1020 | GCA_000153725.1 | 314265 | 344736 | Pelagibaca bermudensis HTCC2601 | HTCC2601 | Proteobacteria | DS022276.1 | III | 2 |
| 1021 | GCA_000153425.1 | 314280 | 74109 | Photobacterium profundum 3TCK | 3TCK | Proteobacteria | CH724134.1 | III | 2 |
| 1022 | GCA_000957245.1 | 61647 | 61647 | Pluralibacter gergoviae | 40874 | Proteobacteria | JZYL01000003.1 | III | 2 |
| 1023 | GCA_001038225.1 | 61647 | 61647 | Pluralibacter gergoviae | JS81F13 | Proteobacteria | LDZF01000024.1 | III | 2 |
| 1024 | GCA_001038285.1 | 61647 | 61647 | Pluralibacter gergoviae | SS84A28 | Proteobacteria | LDZJ01000006.1 | III | 2 |
| 1025 | GCA_001546565.2 | 28125 | 28125 | Prevotella bivia | GED7760C | Bacteroidetes | KQ971199.1 | III | 2 |
| 1026 | GCA_000224595.1 | 563031 | 563031 | Prevotella sp. C561 | C561 | Bacteroidetes | JH114144.1 | III | 2 |
| 1027 | GCA_001463025.1 | 584 | 584 | Proteus mirabilis | 50664164 | Proteobacteria | LNHT01000079.1 | III | 2 |
| 1028 | GCA_001469205.1 | 1761893 | 1761893 | Pseudoalteromonas sp. H103 | H103 | Proteobacteria | LOFG01000015.1 | III | 2 |
| 1029 | GCA_000756575.1 | 287 | 287 | Pseudomonas aeruginosa | ATCC 33988 | Proteobacteria | JPQQ01000086.1 | III | 2 |
| 1030 | GCA_000786565.1 | 287 | 287 | Pseudomonas aeruginosa | PS3 | Proteobacteria | JRGP01000061.1 | III | 2 |
| 1031 | GCA_001420245.1 | 287 | 287 | Pseudomonas aeruginosa | ATCC 33351 | Proteobacteria | LJNX01000028.1 | III | 2 |
| 1032 | GCA_001444915.1 | 287 | 287 | Pseudomonas aeruginosa | ATCC 33364 | Proteobacteria | LJZK01000128.1 | III | 2 |
| 1033 | GCA_001449395.1 | 287 | 287 | Pseudomonas aeruginosa | WH-SGI-V-07066 | Proteobacteria | LLLQ01000001.1 | III | 2 |
| 1034 | GCA_001449485.1 | 287 | 287 | Pseudomonas aeruginosa | WH-SGI-V-07166 | Proteobacteria | LLLV01000015.1 | III | 2 |
| 1035 | GCA_001450095.1 | 287 | 287 | Pseudomonas aeruginosa | WH-SGI-V-07245 | Proteobacteria | LLNQ01000041.1 | III | 2 |
| 1036 | GCA_001450195.1 | 287 | 287 | Pseudomonas aeruginosa | WH-SGI-V-07374 | Proteobacteria | LLNY01000068.1 | III | 2 |
| 1037 | GCA_001450665.1 | 287 | 287 | Pseudomonas aeruginosa | WH-SGI-V-07224 | Proteobacteria | LLMV01000078.1 | III | 2 |
| 1038 | GCA_001450705.1 | 287 | 287 | Pseudomonas aeruginosa | WH-SGI-V-07230 | Proteobacteria | LLNB01000014.1 | III | 2 |
| 1039 | GCA_001450785.1 | 287 | 287 | Pseudomonas aeruginosa | WH-SGI-V-07242 | Proteobacteria | LLNN01000029.1 | III | 2 |
| 1040 | GCA_001450795.1 | 287 | 287 | Pseudomonas aeruginosa | WH-SGI-V-07246 | Proteobacteria | LLNR01000113.1 | III | 2 |
| 1041 | GCA_001450825.1 | 287 | 287 | Pseudomonas aeruginosa | WH-SGI-V-07371 | Proteobacteria | LLNV01000083.1 | III | 2 |
| 1042 | GCA_001450875.1 | 287 | 287 | Pseudomonas aeruginosa | WH-SGI-V-07382 | Proteobacteria | LLOG01000021.1 | III | 2 |
| 1043 | GCA_001451255.1 | 287 | 287 | Pseudomonas aeruginosa | WH-SGI-V-07388 | Proteobacteria | LLOM01000115.1 | III | 2 |
| 1044 | GCA_001451265.1 | 287 | 287 | Pseudomonas aeruginosa | WH-SGI-V-07404 | Proteobacteria | LLOR01000110.1 | III | 2 |
| 1045 | GCA_001451335.1 | 287 | 287 | Pseudomonas aeruginosa | WH-SGI-V-07409 | Proteobacteria | LLOW01000068.1 | III | 2 |
| 1046 | GCA_001451445.1 | 287 | 287 | Pseudomonas aeruginosa | WH-SGI-V-07422 | Proteobacteria | LLPJ01000077.1 | III | 2 |
| 1047 | GCA_001451455.1 | 287 | 287 | Pseudomonas aeruginosa | WH-SGI-V-07424 | Proteobacteria | LLPL01000049.1 | III | 2 |
| 1048 | GCA_001451505.1 | 287 | 287 | Pseudomonas aeruginosa | WH-SGI-V-07426 | Proteobacteria | LLPN01000082.1 | III | 2 |
| 1049 | GCA_001451865.1 | 287 | 287 | Pseudomonas aeruginosa | WH-SGI-V-07502 | Proteobacteria | LLQI01000034.1 | III | 2 |
| 1050 | GCA_001451975.1 | 287 | 287 | Pseudomonas aeruginosa | WH-SGI-V-07621 | Proteobacteria | LLQS01000023.1 | III | 2 |
| 1051 | GCA_001452235.1 | 287 | 287 | Pseudomonas aeruginosa | WH-SGI-V-07642 | Proteobacteria | LLRN01000045.1 | III | 2 |
| 1052 | GCA_001452325.1 | 287 | 287 | Pseudomonas aeruginosa | WH-SGI-V-07647 | Proteobacteria | LLRS01000097.1 | III | 2 |
| 1053 | GCA_001452495.1 | 287 | 287 | Pseudomonas aeruginosa | WH-SGI-V-07689 | Proteobacteria | LLSJ01000050.1 | III | 2 |
| 1054 | GCA_001452565.1 | 287 | 287 | Pseudomonas aeruginosa | WH-SGI-V-07693 | Proteobacteria | LLSN01000128.1 | III | 2 |
| 1055 | GCA_001453025.1 | 287 | 287 | Pseudomonas aeruginosa | WH-SGI-V-07267 | Proteobacteria | LLTY01000116.1 | III | 2 |
| 1056 | GCA_001453065.1 | 287 | 287 | Pseudomonas aeruginosa | WH-SGI-V-07272 | Proteobacteria | LLUA01000034.1 | III | 2 |
| 1057 | GCA_001453235.1 | 287 | 287 | Pseudomonas aeruginosa | WH-SGI-V-07503 | Proteobacteria | LLQJ01000004.1 | III | 2 |
| 1058 | GCA_001453385.1 | 287 | 287 | Pseudomonas aeruginosa | WH-SGI-V-07629 | Proteobacteria | LLRA01000034.1 | III | 2 |
| 1059 | GCA_001453415.1 | 287 | 287 | Pseudomonas aeruginosa | WH-SGI-V-07632 | Proteobacteria | LLRD01000060.1 | III | 2 |
| 1060 | GCA_001453435.1 | 287 | 287 | Pseudomonas aeruginosa | WH-SGI-V-07638 | Proteobacteria | LLRJ01000019.1 | III | 2 |
| 1061 | GCA_001453545.1 | 287 | 287 | Pseudomonas aeruginosa | WH-SGI-V-07678 | Proteobacteria | LLRY01000012.1 | III | 2 |
| 1062 | GCA_001453585.1 | 287 | 287 | Pseudomonas aeruginosa | WH-SGI-V-07682 | Proteobacteria | LLSC01000012.1 | III | 2 |
| 1063 | GCA_001453645.1 | 287 | 287 | Pseudomonas aeruginosa | WH-SGI-V-07686 | Proteobacteria | LLSG01000023.1 | III | 2 |
| 1064 | GCA_001453665.1 | 287 | 287 | Pseudomonas aeruginosa | WH-SGI-V-07691 | Proteobacteria | LLSL01000020.1 | III | 2 |
| 1065 | GCA_001453735.1 | 287 | 287 | Pseudomonas aeruginosa | WH-SGI-V-07701 | Proteobacteria | LLSV01000117.1 | III | 2 |
| 1066 | GCA_001453775.1 | 287 | 287 | Pseudomonas aeruginosa | WH-SGI-V-07710 | Proteobacteria | LLTE01000056.1 | III | 2 |
| 1067 | GCA_001453905.1 | 287 | 287 | Pseudomonas aeruginosa | WH-SGI-V-07273 | Proteobacteria | LLUB01000034.1 | III | 2 |
| 1068 | GCA_001454085.1 | 287 | 287 | Pseudomonas aeruginosa | WH-SGI-V-07284 | Proteobacteria | LLUK01000105.1 | III | 2 |
| 1069 | GCA_001454145.1 | 287 | 287 | Pseudomonas aeruginosa | WH-SGI-V-07291 | Proteobacteria | LLUO01000065.1 | III | 2 |
| 1070 | GCA_001454325.1 | 287 | 287 | Pseudomonas aeruginosa | WH-SGI-V-07310 | Proteobacteria | LLUZ01000067.1 | III | 2 |
| 1071 | GCA_001454415.1 | 287 | 287 | Pseudomonas aeruginosa | WH-SGI-V-07317 | Proteobacteria | LLVF01000026.1 | III | 2 |
| 1072 | GCA_001454725.1 | 287 | 287 | Pseudomonas aeruginosa | WH-SGI-V-07305 | Proteobacteria | LLUW01000034.1 | III | 2 |
| 1073 | GCA_001454745.1 | 287 | 287 | Pseudomonas aeruginosa | WH-SGI-V-07311 | Proteobacteria | LLVA01000045.1 | III | 2 |
| 1074 | GCA_001516245.1 | 287 | 287 | Pseudomonas aeruginosa | F23197 | Proteobacteria | CP008856.1 | III | 2 |
| 1075 | GCA_001516325.1 | 287 | 287 | Pseudomonas aeruginosa | H27930 | Proteobacteria | CP008860.1 | III | 2 |
| 1076 | GCA_001554535.1 | 287 | 287 | Pseudomonas aeruginosa | AU12175 | Proteobacteria | LRYP01000010.1 | III | 2 |
| 1077 | GCA_001554545.1 | 287 | 287 | Pseudomonas aeruginosa | AU13212 | Proteobacteria | LRYQ01000033.1 | III | 2 |
| 1078 | GCA_001554585.1 | 287 | 287 | Pseudomonas aeruginosa | AU15431 | Proteobacteria | LRYV01000009.1 | III | 2 |
| 1079 | GCA_001554615.1 | 287 | 287 | Pseudomonas aeruginosa | AU13213 | Proteobacteria | LRYS01000006.1 | III | 2 |
| 1080 | GCA_001554625.1 | 287 | 287 | Pseudomonas aeruginosa | AU16960 | Proteobacteria | LRYT01000005.1 | III | 2 |
| 1081 | GCA_001555135.1 | 287 | 287 | Pseudomonas aeruginosa | AU7032 | Proteobacteria | LRYU01000008.1 | III | 2 |
| 1082 | GCA_001555145.1 | 287 | 287 | Pseudomonas aeruginosa | AU6923 | Proteobacteria | LRYO01000006.1 | III | 2 |
| 1083 | GCA_001555415.1 | 287 | 287 | Pseudomonas aeruginosa | AU14820 | Proteobacteria | LSAG01000007.1 | III | 2 |
| 1084 | GCA_001555425.1 | 287 | 287 | Pseudomonas aeruginosa | AU23529 | Proteobacteria | LSAI01000005.1 | III | 2 |
| 1085 | GCA_001555455.1 | 287 | 287 | Pseudomonas aeruginosa | AU17965 | Proteobacteria | LSAH01000005.1 | III | 2 |
| 1086 | GCA_001601595.1 | 287 | 287 | Pseudomonas aeruginosa | 105738 | Proteobacteria | LOHK01000062.1 | III | 2 |
| 1087 | GCA_000647595.1 | 1339263 | 287 | Pseudomonas aeruginosa 148 | 148 | Proteobacteria | ATAJ01000107.1 | III | 2 |
| 1088 | GCA_000481105.1 | 1402543 | 287 | Pseudomonas aeruginosa BL02 | BL02 | Proteobacteria | KI518950.1 | III | 2 |
| 1089 | GCA_000481085.1 | 1402544 | 287 | Pseudomonas aeruginosa BL03 | BL03 | Proteobacteria | KI518942.1 | III | 2 |
| 1090 | GCA_000480965.1 | 1402550 | 287 | Pseudomonas aeruginosa BL09 | BL09 | Proteobacteria | KI518886.1 | III | 2 |
| 1091 | GCA_000480905.1 | 1402553 | 287 | Pseudomonas aeruginosa BL12 | BL12 | Proteobacteria | KI518869.1 | III | 2 |
| 1092 | GCA_000480885.1 | 1402554 | 287 | Pseudomonas aeruginosa BL13 | BL13 | Proteobacteria | KI518852.1 | III | 2 |
| 1093 | GCA_000480745.1 | 1402561 | 287 | Pseudomonas aeruginosa BL20 | BL20 | Proteobacteria | KI518796.1 | III | 2 |
| 1094 | GCA_000481485.1 | 1402511 | 287 | Pseudomonas aeruginosa BWHPSA011 | BWHPSA011 | Proteobacteria | KI519089.1 | III | 2 |
| 1095 | GCA_000481245.1 | 1402523 | 287 | Pseudomonas aeruginosa BWHPSA023 | BWHPSA023 | Proteobacteria | KI519011.1 | III | 2 |
| 1096 | GCA_000481145.1 | 1402528 | 287 | Pseudomonas aeruginosa BWHPSA028 | BWHPSA028 | Proteobacteria | KI518967.1 | III | 2 |
| 1097 | GCA_000520415.1 | 1402531 | 287 | Pseudomonas aeruginosa BWHPSA039 | BWHPSA039 | Proteobacteria | KI914530.1 | III | 2 |
| 1098 | GCA_000480515.1 | 1402572 | 287 | Pseudomonas aeruginosa C20 | C20 | Proteobacteria | KI518719.1 | III | 2 |
| 1099 | GCA_000480495.1 | 1402573 | 287 | Pseudomonas aeruginosa C23 | C23 | Proteobacteria | KI518716.1 | III | 2 |
| 1100 | GCA_000705175.1 | 1441459 | 287 | Pseudomonas aeruginosa C2773C | C2773C | Proteobacteria | JGZW01000193.1 | III | 2 |
| 1101 | GCA_000481945.1 | 1402488 | 287 | Pseudomonas aeruginosa CF127 | CF127 | Proteobacteria | KI519290.1 | III | 2 |
| 1102 | GCA_000291745.1 | 1078464 | 287 | Pseudomonas aeruginosa NCMG1179 | NCGM1179 | Proteobacteria | DF126593.1 | III | 2 |
| 1103 | GCA_000342145.1 | 1285581 | 287 | Pseudomonas aeruginosa PA21_ST175 | PA21_ST175 | Proteobacteria | AOIH01000011.1 | III | 2 |
| 1104 | GCA_000611995.2 | 1437873 | 287 | Pseudomonas aeruginosa PA99 | PA99 | Proteobacteria | KK111942.1 | III | 2 |
| 1105 | GCA_000408865.1 | 1009714 | 287 | Pseudomonas aeruginosa PAK | PAK | Proteobacteria | KE137323.1 | III | 2 |
| 1106 | GCA_000520195.1 | 1402581 | 287 | Pseudomonas aeruginosa PS42 | PS42 | Proteobacteria | KI914450.1 | III | 2 |
| 1107 | GCA_000481725.1 | 1402499 | 287 | Pseudomonas aeruginosa S35004 | S35004 | Proteobacteria | KI519208.1 | III | 2 |
| 1108 | GCA_000568215.1 | 1443105 | 287 | Pseudomonas aeruginosa SG17M | SG17M | Proteobacteria | JALF01000002.1 | III | 2 |
| 1109 | GCA_000467105.1 | 1215092 | 43263 | Pseudomonas alcaligenes NBRC 14159 | NBRC 14159 | Proteobacteria | BATI01000047.1 | III | 2 |
| 1110 | GCA_000467105.1 | 1215092 | 43263 | Pseudomonas alcaligenes NBRC 14159 | NBRC 14159 | Proteobacteria | BATI01000005.1 | III | 2 |
| 1111 | GCA_000632535.1 | 653930 | 653930 | Pseudomonas bauzanensis | W13Z2 | Proteobacteria | JFHS01000003.1 | III | 2 |
| 1112 | GCA_001586155.1 | 53408 | 53408 | Pseudomonas citronellolis | P3B5 | Proteobacteria | CP014158.1 | III | 2 |
| 1113 | GCA_001400695.1 | 129135 | 53409 | Pseudomonas coronafaciens pv. atropurpurea | ICMP4457 | Proteobacteria | LJPS01000041.1 | III | 2 |
| 1114 | GCA_001411475.1 | 1563157 | 1563157 | Pseudomonas endophytica | BSTT44 | Proteobacteria | LLWH01000162.1 | III | 2 |
| 1115 | GCA_000242115.2 | 1117958 | 359110 | Pseudomonas extremaustralis 14-3 substr. 14-3b | 14-3 substr. 14-3b | Proteobacteria | AHIP01000001.1 | III | 2 |
| 1116 | GCA_000876155.1 | 294 | 294 | Pseudomonas fluorescens | 2-79 | Proteobacteria | JXCQ01000014.1 | III | 2 |
| 1117 | GCA_000952735.1 | 294 | 294 | Pseudomonas fluorescens | NT0133 | Proteobacteria | JYHW01000034.1 | III | 2 |
| 1118 | GCA_001542725.1 | 294 | 294 | Pseudomonas fluorescens | Ps_40 | Proteobacteria | LCYC01000002.1 | III | 2 |
| 1119 | GCA_000166515.1 | 746360 | 294 | Pseudomonas fluorescens WH6 | WH6 | Proteobacteria | CM001025.1 | III | 2 |
| 1120 | GCA_001043005.1 | 122355 | 122355 | Pseudomonas psychrophila | DSM 17535 | Proteobacteria | JYKZ01000009.1 | III | 2 |
| 1121 | GCA_000367825.1 | 1227924 | 303 | Pseudomonas putida TRO1 | TRO1 | Proteobacteria | APBQ01000056.1 | III | 2 |
| 1122 | GCA_000478505.2 | 1203578 | 1203578 | Pseudomonas sp. HPB0071 | HPB0071 | Proteobacteria | KI517369.1 | III | 2 |
| 1123 | GCA_000307775.2 | 1218352 | 316 | Pseudomonas stutzeri KOS6 | KOS6 | Proteobacteria | KK020676.1 | III | 2 |
| 1124 | GCA_000341615.1 | 1212548 | 316 | Pseudomonas stutzeri NF13 | NF13 | Proteobacteria | AOBS01000069.1 | III | 2 |
| 1125 | GCA_001293855.1 | 59511 | 251701 | Pseudomonas syringae pv. maculicola | 90_32 | Proteobacteria | LGLH01000088.1 | III | 2 |
| 1126 | GCA_000735435.1 | 1005996 | 575 | Raoultella planticola ATCC 33531 | ATCC 33531 | Proteobacteria | JMPP01000005.1 | III | 2 |
| 1127 | GCA_000020265.1 | 491916 | 29449 | Rhizobium etli CIAT 652 | CIAT 652 | Proteobacteria | CP001074.1 | III | 2 |
| 1128 | GCA_000268285.2 | 1128399 | 396 | Rhizobium phaseoli Ch24-10 | Ch24-10 | Proteobacteria | AHJU02000014.1 | III | 2 |
| 1129 | GCA_001425605.1 | 1736380 | 1736380 | Rhizobium sp. Leaf453 | Leaf453 | Proteobacteria | LMRG01000007.1 | III | 2 |
| 1130 | GCA_000967425.1 | 1125847 | 1125847 | Rhizobium sp. NT-26 | NT-26 | Proteobacteria | FO082820.1 | III | 2 |
| 1131 | GCA_001427345.1 | 1736473 | 1736473 | Rhizobium sp. Root149 | Root149 | Proteobacteria | LMGD01000001.1 | III | 2 |
| 1132 | GCA_000931365.1 | 1604021 | 1604021 | Rhizobium sp. UR51a | UR51a | Proteobacteria | JYFU01000014.1 | III | 2 |
| 1133 | GCA_001402715.1 | 1061 | 1061 | Rhodobacter capsulatus | B41 | Proteobacteria | LLVU01000015.1 | III | 2 |
| 1134 | GCA_001402725.1 | 1061 | 1061 | Rhodobacter capsulatus | A52 | Proteobacteria | LLVV01000015.1 | III | 2 |
| 1135 | GCA_000521785.1 | 1449350 | 93683 | Roseivivax halodurans JCM 10272 | JCM 10272 | Proteobacteria | JALZ01000004.1 | III | 2 |
| 1136 | GCA_000961715.1 | 1629709 | 1629709 | Roseovarius sp. BRH_c41 | | Proteobacteria | LADY01000060.1 | III | 2 |
| 1137 | GCA_000442315.1 | 1123069 | 295419 | Rubellimicrobium thermophilum DSM 16684 | DSM 16684 | Proteobacteria | KE557322.1 | III | 2 |
| 1138 | GCA_001570325.1 | 28901 | 28901 | Salmonella enterica | NGUA40 | Proteobacteria | BCPM01000156.1 | III | 2 |
| 1139 | GCA_001570345.1 | 28901 | 28901 | Salmonella enterica | NGUA41 | Proteobacteria | BCPN01000199.1 | III | 2 |
| 1140 | GCA_000806705.1 | 59201 | 28901 | Salmonella enterica subsp. enterica | CFSAN024770 | Proteobacteria | JWQJ01000002.1 | III | 2 |
| 1141 | GCA_000806725.1 | 59201 | 28901 | Salmonella enterica subsp. enterica | CFSAN024771 | Proteobacteria | JWQI01000002.1 | III | 2 |
| 1142 | GCA_000487735.1 | 1194156 | 28901 | Salmonella enterica subsp. enterica serovar Havana str. | CFSAN001082 | Proteobacteria | APAH01000409.1 | III | 2 |
| | | | | CFSAN001082 | | | | | |
| 1143 | GCA_001187365.1 | 595 | 28901 | Salmonella enterica subsp. enterica serovar Infantis | Sal280 | Proteobacteria | LFGW01000205.1 | III | 2 |
| 1144 | GCA_000818075.1 | 54388 | 28901 | Salmonella enterica subsp. enterica serovar Paratyphi A | CMCC 50973 | Proteobacteria | CP009049.1 | III | 2 |
| 1145 | GCA_000818115.1 | 54388 | 28901 | Salmonella enterica subsp. enterica serovar Paratyphi A | CMCC 50503 | Proteobacteria | CP009559.1 | III | 2 |
| 1146 | GCA_000983135.1 | 54388 | 28901 | Salmonella enterica subsp. enterica serovar Paratyphi A | | Proteobacteria | CCTC01000001.1 | III | 2 |
| 1147 | GCA_000983155.1 | 54388 | 28901 | Salmonella enterica subsp. enterica serovar Paratyphi A | | Proteobacteria | CCTP01000001.1 | III | 2 |
| 1148 | GCA_000983175.1 | 54388 | 28901 | Salmonella enterica subsp. enterica serovar Paratyphi A | | Proteobacteria | CCLD01000002.1 | III | 2 |
| 1149 | GCA_000983195.1 | 54388 | 28901 | Salmonella enterica subsp. enterica serovar Paratyphi A | | Proteobacteria | CCTT01000001.1 | III | 2 |
| 1150 | GCA_000983215.1 | 54388 | 28901 | Salmonella enterica subsp. enterica serovar Paratyphi A | | Proteobacteria | CCVA01000001.1 | III | 2 |
| 1151 | GCA_000983235.1 | 54388 | 28901 | Salmonella enterica subsp. enterica serovar Paratyphi A | | Proteobacteria | CCUS01000001.1 | III | 2 |
| 1152 | GCA_000983255.1 | 54388 | 28901 | Salmonella enterica subsp. enterica serovar Paratyphi A | | Proteobacteria | CCNZ01000001.1 | III | 2 |
| 1153 | GCA_000983275.1 | 54388 | 28901 | Salmonella enterica subsp. enterica serovar Paratyphi A | | Proteobacteria | CCVV01000001.1 | III | 2 |
| 1154 | GCA_000983295.1 | 54388 | 28901 | Salmonella enterica subsp. enterica serovar Paratyphi A | | Proteobacteria | CCLR01000001.1 | III | 2 |
| 1155 | GCA_000983315.1 | 54388 | 28901 | Salmonella enterica subsp. enterica serovar Paratyphi A | | Proteobacteria | CGIE01000004.1 | III | 2 |
| 1156 | GCA_000983335.1 | 54388 | 28901 | Salmonella enterica subsp. enterica serovar Paratyphi A | | Proteobacteria | CCNY01000001.1 | III | 2 |
| 1157 | GCA_000983355.1 | 54388 | 28901 | Salmonella enterica subsp. enterica serovar Paratyphi A | | Proteobacteria | CCUY01000004.1 | III | 2 |
| 1158 | GCA_000983375.1 | 54388 | 28901 | Salmonella enterica subsp. enterica serovar Paratyphi A | | Proteobacteria | CCLN01000001.1 | III | 2 |
| 1159 | GCA_000983395.1 | 54388 | 28901 | Salmonella enterica subsp. enterica serovar Paratyphi A | | Proteobacteria | CCTO01000001.1 | III | 2 |
| 1160 | GCA_000983415.1 | 54388 | 28901 | Salmonella enterica subsp. enterica serovar Paratyphi A | | Proteobacteria | CCUE01000001.1 | III | 2 |
| 1161 | GCA_000983435.1 | 54388 | 28901 | Salmonella enterica subsp. enterica serovar Paratyphi A | | Proteobacteria | CCSP01000001.1 | III | 2 |
| 1162 | GCA_000983485.1 | 54388 | 28901 | Salmonella enterica subsp. enterica serovar Paratyphi A | | Proteobacteria | CCLS01000001.1 | III | 2 |
| 1163 | GCA_000983505.1 | 54388 | 28901 | Salmonella enterica subsp. enterica serovar Paratyphi A | | Proteobacteria | CCSN01000001.1 | III | 2 |
| 1164 | GCA_000983535.1 | 54388 | 28901 | Salmonella enterica subsp. enterica serovar Paratyphi A | | Proteobacteria | CCVL01000001.1 | III | 2 |
| 1165 | GCA_000983565.1 | 54388 | 28901 | Salmonella enterica subsp. enterica serovar Paratyphi A | | Proteobacteria | CCUC01000004.1 | III | 2 |
| 1166 | GCA_000983595.1 | 54388 | 28901 | Salmonella enterica subsp. enterica serovar Paratyphi A | | Proteobacteria | CCNT01000003.1 | III | 2 |
| 1167 | GCA_000983625.1 | 54388 | 28901 | Salmonella enterica subsp. enterica serovar Paratyphi A | | Proteobacteria | CCUP01000001.1 | III | 2 |
| 1168 | GCA_000983645.1 | 54388 | 28901 | Salmonella enterica subsp. enterica serovar Paratyphi A | | Proteobacteria | CCLA01000004.1 | III | 2 |
| 1169 | GCA_000983685.1 | 54388 | 28901 | Salmonella enterica subsp. enterica serovar Paratyphi A | | Proteobacteria | CCLI01000001.1 | III | 2 |
| 1170 | GCA_000983705.1 | 54388 | 28901 | Salmonella enterica subsp. enterica serovar Paratyphi A | | Proteobacteria | CCKR01000004.1 | III | 2 |
| 1171 | GCA_000983735.1 | 54388 | 28901 | Salmonella enterica subsp. enterica serovar Paratyphi A | | Proteobacteria | CCTA01000001.1 | III | 2 |
| 1172 | GCA_000983755.1 | 54388 | 28901 | Salmonella enterica subsp. enterica serovar Paratyphi A | | Proteobacteria | CCLQ01000001.1 | III | 2 |
| 1173 | GCA_000983775.1 | 54388 | 28901 | Salmonella enterica subsp. enterica serovar Paratyphi A | | Proteobacteria | CCSR01000004.1 | III | 2 |
| 1174 | GCA_000983795.1 | 54388 | 28901 | Salmonella enterica subsp. enterica serovar Paratyphi A | | Proteobacteria | CCKX01000001.1 | III | 2 |
| 1175 | GCA_000983835.1 | 54388 | 28901 | Salmonella enterica subsp. enterica serovar Paratyphi A | | Proteobacteria | CCLZ01000001.1 | III | 2 |
| 1176 | GCA_000983855.1 | 54388 | 28901 | Salmonella enterica subsp. enterica serovar Paratyphi A | | Proteobacteria | CCUI01000001.1 | III | 2 |
| 1177 | GCA_000983875.1 | 54388 | 28901 | Salmonella enterica subsp. enterica serovar Paratyphi A | | Proteobacteria | CCTL01000001.1 | III | 2 |
| 1178 | GCA_000983895.1 | 54388 | 28901 | Salmonella enterica subsp. enterica serovar Paratyphi A | | Proteobacteria | CCUH01000001.1 | III | 2 |
| 1179 | GCA_000983915.1 | 54388 | 28901 | Salmonella enterica subsp. enterica serovar Paratyphi A | | Proteobacteria | CCUJ01000001.1 | III | 2 |
| 1180 | GCA_000983935.1 | 54388 | 28901 | Salmonella enterica subsp. enterica serovar Paratyphi A | | Proteobacteria | CCUV01000001.1 | III | 2 |
| 1181 | GCA_000983955.1 | 54388 | 28901 | Salmonella enterica subsp. enterica serovar Paratyphi A | | Proteobacteria | CCUM01000001.1 | III | 2 |
| 1182 | GCA_000983975.1 | 54388 | 28901 | Salmonella enterica subsp. enterica serovar Paratyphi A | | Proteobacteria | CCLM01000001.1 | III | 2 |
| 1183 | GCA_000983995.1 | 54388 | 28901 | Salmonella enterica subsp. enterica serovar Paratyphi A | | Proteobacteria | CCMD01000001.1 | III | 2 |
| 1184 | GCA_000984025.1 | 54388 | 28901 | Salmonella enterica subsp. enterica serovar Paratyphi A | | Proteobacteria | CCTI01000001.1 | III | 2 |
| 1185 | GCA_000984045.1 | 54388 | 28901 | Salmonella enterica subsp. enterica serovar Paratyphi A | | Proteobacteria | CCVG01000001.1 | III | 2 |
| 1186 | GCA_000984075.1 | 54388 | 28901 | Salmonella enterica subsp. enterica serovar Paratyphi A | | Proteobacteria | CCSV01000001.1 | III | 2 |
| 1187 | GCA_000984095.1 | 54388 | 28901 | Salmonella enterica subsp. enterica serovar Paratyphi A | | Proteobacteria | CCLX01000001.1 | III | 2 |
| 1188 | GCA_000984115.1 | 54388 | 28901 | Salmonella enterica subsp. enterica serovar Paratyphi A | | Proteobacteria | CCUD01000001.1 | III | 2 |
| 1189 | GCA_000984135.1 | 54388 | 28901 | Salmonella enterica subsp. enterica serovar Paratyphi A | | Proteobacteria | CCUX01000001.1 | III | 2 |
| 1190 | GCA_000984175.1 | 54388 | 28901 | Salmonella enterica subsp. enterica serovar Paratyphi A | | Proteobacteria | CCTZ01000003.1 | III | 2 |
| 1191 | GCA_000984195.1 | 54388 | 28901 | Salmonella enterica subsp. enterica serovar Paratyphi A | | Proteobacteria | CCML01000001.1 | III | 2 |
| 1192 | GCA_000984215.1 | 54388 | 28901 | Salmonella enterica subsp. enterica serovar Paratyphi A | | Proteobacteria | CCTN01000001.1 | III | 2 |
| 1193 | GCA_000984235.1 | 54388 | 28901 | Salmonella enterica subsp. enterica serovar Paratyphi A | | Proteobacteria | CCUB01000001.1 | III | 2 |
| 1194 | GCA_000984255.1 | 54388 | 28901 | Salmonella enterica subsp. enterica serovar Paratyphi A | | Proteobacteria | CCUR01000001.1 | III | 2 |
| 1195 | GCA_000984275.1 | 54388 | 28901 | Salmonella enterica subsp. enterica serovar Paratyphi A | | Proteobacteria | CCVD01000005.1 | III | 2 |
| 1196 | GCA_000984295.1 | 54388 | 28901 | Salmonella enterica subsp. enterica serovar Paratyphi A | | Proteobacteria | CCKT01000001.1 | III | 2 |
| 1197 | GCA_000984315.1 | 54388 | 28901 | Salmonella enterica subsp. enterica serovar Paratyphi A | | Proteobacteria | CCUO01000002.1 | III | 2 |
| 1198 | GCA_000984335.1 | 54388 | 28901 | Salmonella enterica subsp. enterica serovar Paratyphi A | | Proteobacteria | CCME01000001.1 | III | 2 |
| 1199 | GCA_000984355.1 | 54388 | 28901 | Salmonella enterica subsp. enterica serovar Paratyphi A | | Proteobacteria | CCVJ01000005.1 | III | 2 |
| 1200 | GCA_000984375.1 | 54388 | 28901 | Salmonella enterica subsp. enterica serovar Paratyphi A | | Proteobacteria | CCSY01000004.1 | III | 2 |
| 1201 | GCA_000984395.1 | 54388 | 28901 | Salmonella enterica subsp. enterica serovar Paratyphi A | | Proteobacteria | CCUU01000001.1 | III | 2 |
| 1202 | GCA_000984415.1 | 54388 | 28901 | Salmonella enterica subsp. enterica serovar Paratyphi A | | Proteobacteria | CCMA01000001.1 | III | 2 |
| 1203 | GCA_000984435.1 | 54388 | 28901 | Salmonella enterica subsp. enterica serovar Paratyphi A | | Proteobacteria | CCSU01000001.1 | III | 2 |
| 1204 | GCA_000984455.1 | 54388 | 28901 | Salmonella enterica subsp. enterica serovar Paratyphi A | | Proteobacteria | CCKS01000001.1 | III | 2 |
| 1205 | GCA_000984475.1 | 54388 | 28901 | Salmonella enterica subsp. enterica serovar Paratyphi A | | Proteobacteria | CCVF01000001.1 | III | 2 |
| 1206 | GCA_000984495.1 | 54388 | 28901 | Salmonella enterica subsp. enterica serovar Paratyphi A | | Proteobacteria | CCST01000001.1 | III | 2 |
| 1207 | GCA_000984515.1 | 54388 | 28901 | Salmonella enterica subsp. enterica serovar Paratyphi A | | Proteobacteria | CCKU01000001.1 | III | 2 |
| 1208 | GCA_000984535.1 | 54388 | 28901 | Salmonella enterica subsp. enterica serovar Paratyphi A | | Proteobacteria | CCUL01000001.1 | III | 2 |
| 1209 | GCA_000984555.1 | 54388 | 28901 | Salmonella enterica subsp. enterica serovar Paratyphi A | | Proteobacteria | CCKY01000002.1 | III | 2 |
| 1210 | GCA_000984575.1 | 54388 | 28901 | Salmonella enterica subsp. enterica serovar Paratyphi A | | Proteobacteria | CCUG01000001.1 | III | 2 |
| 1211 | GCA_000984595.1 | 54388 | 28901 | Salmonella enterica subsp. enterica serovar Paratyphi A | | Proteobacteria | CCLG01000001.1 | III | 2 |
| 1212 | GCA_000984615.1 | 54388 | 28901 | Salmonella enterica subsp. enterica serovar Paratyphi A | | Proteobacteria | CCLO01000005.1 | III | 2 |
| 1213 | GCA_000984635.1 | 54388 | 28901 | Salmonella enterica subsp. enterica serovar Paratyphi A | | Proteobacteria | CCTM01000001.1 | III | 2 |
| 1214 | GCA_000984655.1 | 54388 | 28901 | Salmonella enterica subsp. enterica serovar Paratyphi A | | Proteobacteria | CCKZ01000001.1 | III | 2 |
| 1215 | GCA_000984675.1 | 54388 | 28901 | Salmonella enterica subsp. enterica serovar Paratyphi A | | Proteobacteria | CCTJ01000001.1 | III | 2 |
| 1216 | GCA_000984695.1 | 54388 | 28901 | Salmonella enterica subsp. enterica serovar Paratyphi A | | Proteobacteria | CCSW01000001.1 | III | 2 |
| 1217 | GCA_000984715.1 | 54388 | 28901 | Salmonella enterica subsp. enterica serovar Paratyphi A | | Proteobacteria | CCVH01000001.1 | III | 2 |
| 1218 | GCA_000984735.1 | 54388 | 28901 | Salmonella enterica subsp. enterica serovar Paratyphi A | | Proteobacteria | CCKW01000001.1 | III | 2 |
| 1219 | GCA_000984755.1 | 54388 | 28901 | Salmonella enterica subsp. enterica serovar Paratyphi A | | Proteobacteria | CCTE01000001.1 | III | 2 |
| 1220 | GCA_000984795.1 | 54388 | 28901 | Salmonella enterica subsp. enterica serovar Paratyphi A | | Proteobacteria | CCLJ01000001.1 | III | 2 |
| 1221 | GCA_000984815.1 | 54388 | 28901 | Salmonella enterica subsp. enterica serovar Paratyphi A | | Proteobacteria | CCSX01000002.1 | III | 2 |
| 1222 | GCA_000984835.1 | 54388 | 28901 | Salmonella enterica subsp. enterica serovar Paratyphi A | | Proteobacteria | CCTW01000001.1 | III | 2 |
| 1223 | GCA_000984855.1 | 54388 | 28901 | Salmonella enterica subsp. enterica serovar Paratyphi A | | Proteobacteria | CCLU01000001.1 | III | 2 |
| 1224 | GCA_000984875.1 | 54388 | 28901 | Salmonella enterica subsp. enterica serovar Paratyphi A | | Proteobacteria | CCTK01000001.1 | III | 2 |
| 1225 | GCA_000984895.1 | 54388 | 28901 | Salmonella enterica subsp. enterica serovar Paratyphi A | | Proteobacteria | CCNU01000001.1 | III | 2 |
| 1226 | GCA_000984915.1 | 54388 | 28901 | Salmonella enterica subsp. enterica serovar Paratyphi A | | Proteobacteria | CCNV01000001.1 | III | 2 |
| 1227 | GCA_000984935.1 | 54388 | 28901 | Salmonella enterica subsp. enterica serovar Paratyphi A | | Proteobacteria | CCLW01000001.1 | III | 2 |
| 1228 | GCA_000984955.1 | 54388 | 28901 | Salmonella enterica subsp. enterica serovar Paratyphi A | | Proteobacteria | CCLY01000001.1 | III | 2 |
| 1229 | GCA_000984975.1 | 54388 | 28901 | Salmonella enterica subsp. enterica serovar Paratyphi A | | Proteobacteria | CCLL01000004.1 | III | 2 |
| 1230 | GCA_000984995.1 | 54388 | 28901 | Salmonella enterica subsp. enterica serovar Paratyphi A | | Proteobacteria | CCSM01000001.1 | III | 2 |
| 1231 | GCA_000985015.1 | 54388 | 28901 | Salmonella enterica subsp. enterica serovar Paratyphi A | | Proteobacteria | CCUF01000001.1 | III | 2 |
| 1232 | GCA_000985035.1 | 54388 | 28901 | Salmonella enterica subsp. enterica serovar Paratyphi A | | Proteobacteria | CCLT01000001.1 | III | 2 |
| 1233 | GCA_000985055.1 | 54388 | 28901 | Salmonella enterica subsp. enterica serovar Paratyphi A | | Proteobacteria | CCMC01000001.1 | III | 2 |
| 1234 | GCA_000985075.1 | 54388 | 28901 | Salmonella enterica subsp. enterica serovar Paratyphi A | | Proteobacteria | CCUZ01000001.1 | III | 2 |
| 1235 | GCA_000985095.1 | 54388 | 28901 | Salmonella enterica subsp. enterica serovar Paratyphi A | | Proteobacteria | CCLF01000001.1 | III | 2 |
| 1236 | GCA_000985115.1 | 54388 | 28901 | Salmonella enterica subsp. enterica serovar Paratyphi A | | Proteobacteria | CCTU01000001.1 | III | 2 |
| 1237 | GCA_000985135.1 | 54388 | 28901 | Salmonella enterica subsp. enterica serovar Paratyphi A | | Proteobacteria | CCUN01000001.1 | III | 2 |
| 1238 | GCA_000985155.1 | 54388 | 28901 | Salmonella enterica subsp. enterica serovar Paratyphi A | | Proteobacteria | CCLE01000001.1 | III | 2 |
| 1239 | GCA_000985175.1 | 54388 | 28901 | Salmonella enterica subsp. enterica serovar Paratyphi A | | Proteobacteria | CCTR01000001.1 | III | 2 |
| 1240 | GCA_000985195.1 | 54388 | 28901 | Salmonella enterica subsp. enterica serovar Paratyphi A | | Proteobacteria | CCUW01000001.1 | III | 2 |
| 1241 | GCA_000985215.1 | 54388 | 28901 | Salmonella enterica subsp. enterica serovar Paratyphi A | | Proteobacteria | CCLP01000001.1 | III | 2 |
| 1242 | GCA_000985235.1 | 54388 | 28901 | Salmonella enterica subsp. enterica serovar Paratyphi A | | Proteobacteria | CCLK01000001.1 | III | 2 |
| 1243 | GCA_000985255.1 | 54388 | 28901 | Salmonella enterica subsp. enterica serovar Paratyphi A | | Proteobacteria | CCSS01000001.1 | III | 2 |
| 1244 | GCA_000985275.1 | 54388 | 28901 | Salmonella enterica subsp. enterica serovar Paratyphi A | | Proteobacteria | CCSO01000001.1 | III | 2 |
| 1245 | GCA_000985295.1 | 54388 | 28901 | Salmonella enterica subsp. enterica serovar Paratyphi A | | Proteobacteria | CCUT01000001.1 | III | 2 |
| 1246 | GCA_000985315.1 | 54388 | 28901 | Salmonella enterica subsp. enterica serovar Paratyphi A | | Proteobacteria | CCTQ01000001.1 | III | 2 |
| 1247 | GCA_000985335.1 | 54388 | 28901 | Salmonella enterica subsp. enterica serovar Paratyphi A | | Proteobacteria | CCVC01000001.1 | III | 2 |
| 1248 | GCA_000985355.1 | 54388 | 28901 | Salmonella enterica subsp. enterica serovar Paratyphi A | | Proteobacteria | CCVE01000001.1 | III | 2 |
| 1249 | GCA_000985375.1 | 54388 | 28901 | Salmonella enterica subsp. enterica serovar Paratyphi A | | Proteobacteria | CCTG01000001.1 | III | 2 |
| 1250 | GCA_000985395.1 | 54388 | 28901 | Salmonella enterica subsp. enterica serovar Paratyphi A | | Proteobacteria | CCSL01000001.1 | III | 2 |
| 1251 | GCA_000985415.1 | 54388 | 28901 | Salmonella enterica subsp. enterica serovar Paratyphi A | | Proteobacteria | CCVB01000003.1 | III | 2 |
| 1252 | GCA_000985435.1 | 54388 | 28901 | Salmonella enterica subsp. enterica serovar Paratyphi A | | Proteobacteria | CCUA01000001.1 | III | 2 |
| 1253 | GCA_000985455.1 | 54388 | 28901 | Salmonella enterica subsp. enterica serovar Paratyphi A | | Proteobacteria | CCNX01000001.1 | III | 2 |
| 1254 | GCA_000985475.1 | 54388 | 28901 | Salmonella enterica subsp. enterica serovar Paratyphi A | | Proteobacteria | CCSZ01000004.1 | III | 2 |
| 1255 | GCA_000985495.1 | 54388 | 28901 | Salmonella enterica subsp. enterica serovar Paratyphi A | | Proteobacteria | CCTD01000002.1 | III | 2 |
| 1256 | GCA_000985515.1 | 54388 | 28901 | Salmonella enterica subsp. enterica serovar Paratyphi A | | Proteobacteria | CCVI01000001.1 | III | 2 |
| 1257 | GCA_000985535.1 | 54388 | 28901 | Salmonella enterica subsp. enterica serovar Paratyphi A | | Proteobacteria | CCUK01000001.1 | III | 2 |
| 1258 | GCA_000985555.1 | 54388 | 28901 | Salmonella enterica subsp. enterica serovar Paratyphi A | | Proteobacteria | CCTX01000003.1 | III | 2 |
| 1259 | GCA_000985575.1 | 54388 | 28901 | Salmonella enterica subsp. enterica serovar Paratyphi A | | Proteobacteria | CCTF01000001.1 | III | 2 |
| 1260 | GCA_000985595.1 | 54388 | 28901 | Salmonella enterica subsp. enterica serovar Paratyphi A | | Proteobacteria | CCUQ01000001.1 | III | 2 |
| 1261 | GCA_000985615.1 | 54388 | 28901 | Salmonella enterica subsp. enterica serovar Paratyphi A | | Proteobacteria | CCMF01000001.1 | III | 2 |
| 1262 | GCA_000985655.1 | 54388 | 28901 | Salmonella enterica subsp. enterica serovar Paratyphi A | | Proteobacteria | CCTY01000001.1 | III | 2 |
| 1263 | GCA_000985675.1 | 54388 | 28901 | Salmonella enterica subsp. enterica serovar Paratyphi A | | Proteobacteria | CCKV01000001.1 | III | 2 |
| 1264 | GCA_000985695.1 | 54388 | 28901 | Salmonella enterica subsp. enterica serovar Paratyphi A | | Proteobacteria | CCTV01000001.1 | III | 2 |
| 1265 | GCA_000985715.1 | 54388 | 28901 | Salmonella enterica subsp. enterica serovar Paratyphi A | | Proteobacteria | CCVK01000001.1 | III | 2 |
| 1266 | GCA_000985735.1 | 54388 | 28901 | Salmonella enterica subsp. enterica serovar Paratyphi A | | Proteobacteria | CCMB01000001.1 | III | 2 |
| 1267 | GCA_000985755.1 | 54388 | 28901 | Salmonella enterica subsp. enterica serovar Paratyphi A | | Proteobacteria | CCLH01000001.1 | III | 2 |
| 1268 | GCA_000985775.1 | 54388 | 28901 | Salmonella enterica subsp. enterica serovar Paratyphi A | | Proteobacteria | CCSQ01000001.1 | III | 2 |
| 1269 | GCA_000985795.1 | 54388 | 28901 | Salmonella enterica subsp. enterica serovar Paratyphi A | | Proteobacteria | CCTH01000001.1 | III | 2 |
| 1270 | GCA_000985815.1 | 54388 | 28901 | Salmonella enterica subsp. enterica serovar Paratyphi A | | Proteobacteria | CCSK01000001.1 | III | 2 |
| 1271 | GCA_000985835.1 | 54388 | 28901 | Salmonella enterica subsp. enterica serovar Paratyphi A | | Proteobacteria | CCNW01000001.1 | III | 2 |
| 1272 | GCA_000985855.1 | 54388 | 28901 | Salmonella enterica subsp. enterica serovar Paratyphi A | | Proteobacteria | CCTB01000001.1 | III | 2 |
| 1273 | GCA_001045395.1 | 54388 | 28901 | Salmonella enterica subsp. enterica serovar Paratyphi A | CMCC50093 | Proteobacteria | CP011967.1 | III | 2 |
| 1274 | GCA_000026565.1 | 554290 | 28901 | Salmonella enterica subsp. enterica serovar Paratyphi A str. | AKU12601 | Proteobacteria | FM200053.1 | III | 2 |
| | | | | AKU_12601 | | | | | |
| 1275 | GCA_000486725.1 | 941189 | 28901 | Salmonella enterica subsp. enterica serovar Paratyphi A str. | ATCC 11511 | Proteobacteria | AOYH01000034.1 | III | 2 |
| | | | | ATCC 11511 | | | | | |
| 1276 | GCA_000011885.1 | 295319 | 28901 | Salmonella enterica subsp. enterica serovar Paratyphi A str. | ATCC 9150 | Proteobacteria | CP000026.1 | III | 2 |
| | | | | ATCC 9150 | | | | | |
| 1277 | GCA_000411815.1 | 1072568 | 28901 | Salmonella enterica subsp. enterica serovar Paratyphi A str. | GXS2268 | Proteobacteria | AFYX01000025.1 | III | 2 |
| | | | | GXS2268 | | | | | |
| 1278 | GCA_000411795.1 | 1072569 | 28901 | Salmonella enterica subsp. enterica serovar Paratyphi A str. | GZ9A00052 | Proteobacteria | AFYW01000030.1 | III | 2 |
| | | | | GZ9A00052 | | | | | |
| 1279 | GCA_000411835.1 | 1072570 | 28901 | Salmonella enterica subsp. enterica serovar Paratyphi A str. JX05- | JX05-19 | Proteobacteria | AFYY01000027.1 | III | 2 |
| | | | | 19 | | | | | |
| 1280 | GCA_001448825.1 | 1243588 | 28901 | Salmonella enterica subsp. enterica serovar Paratyphi A str. | SA19950809 | Proteobacteria | LHTE01000005.1 | III | 2 |
| | | | | SA19950809 | | | | | |
| 1281 | GCA_000335835.1 | 687914 | 28901 | Salmonella enterica subsp. enterica serovar Rissen str. 150 | 150 | Proteobacteria | AHUI01000053.1 | III | 2 |
| 1282 | GCA_000231685.2 | 913082 | 28901 | Salmonella enterica subsp. enterica serovar Senftenberg str. A4- | A4-543 | Proteobacteria | AFCU01002067.1 | III | 2 |
| | | | | 543 | | | | | |
| 1283 | GCA_000486525.1 | 930772 | 28901 | Salmonella enterica subsp. enterica serovar Senftenberg str. | ATCC 43845 | Proteobacteria | AOXX01000100.1 | III | 2 |
| | | | | ATCC 43845 | | | | | |
| 1284 | GCA_001409135.1 | 57743 | 28901 | Salmonella enterica subsp. enterica serovar Weltevreden | 2511STDY5462388 | Proteobacteria | LN890518.1 | III | 2 |
| 1285 | GCA_001409155.1 | 57743 | 28901 | Salmonella enterica subsp. enterica serovar Weltevreden | 2511STDY5712384 | Proteobacteria | LN890522.1 | III | 2 |
| 1286 | GCA_001409175.1 | 57743 | 28901 | Salmonella enterica subsp. enterica serovar Weltevreden | 2511STDY5712385 | Proteobacteria | LN890524.1 | III | 2 |
| 1287 | GCA_001409195.1 | 57743 | 28901 | Salmonella enterica subsp. enterica serovar Weltevreden | 2511STDY5462413 | Proteobacteria | LN890520.1 | III | 2 |
| 1288 | GCA_000230505.1 | 936157 | 28901 | Salmonella enterica subsp. enterica serovar Weltevreden str. | 2007-60-3289-1 | Proteobacteria | FR775224.1 | III | 2 |
| | | | | 2007-60-3289-1 | | | | | |
| 1289 | GCA_000171495.1 | 465518 | 28901 | Salmonella enterica subsp. enterica serovar Weltevreden str. | HI_N05-537 | Proteobacteria | ABFF01000010.1 | III | 2 |
| | | | | HI_N05-537 | | | | | |
| 1290 | GCA_001241965.1 | 1160769 | 28901 | Salmonella enterica subsp. enterica serovar Worthingtor | CVM N45406 | Proteobacteria | LHGK01000019.1 | III | 2 |
| 1291 | GCA_001242245.1 | 1160769 | 28901 | Salmonella enterica subsp. enterica serovar Worthingtor | CVM N45931 | Proteobacteria | LHGV01000001.1 | III | 2 |
| 1292 | GCA_001243275.1 | 1160769 | 28901 | Salmonella enterica subsp. enterica serovar Worthingtor | CVM N46835 | Proteobacteria | LHIL01000018.1 | III | 2 |
| 1293 | GCA_001247285.1 | 1160769 | 28901 | Salmonella enterica subsp. enterica serovar Worthingtor | CVM N51304 | Proteobacteria | LHOK01000001.1 | III | 2 |
| 1294 | GCA_001479505.1 | 1160769 | 28901 | Salmonella enterica subsp. enterica serovar Worthingtor | CVM N32773 | Proteobacteria | JYWH01000045.1 | III | 2 |
| 1295 | GCA_001480165.1 | 1160769 | 28901 | Salmonella enterica subsp. enterica serovar Worthingtor | CVM N40374 | Proteobacteria | JYYI01000001.1 | III | 2 |
| 1296 | GCA_000486265.1 | 1173940 | 28901 | Salmonella enterica subsp. enterica serovar Worthington str. | ATCC 9607 | Proteobacteria | AOXK01000084.1 | III | 2 |
| | | | | ATCC 9607 | | | | | |
| 1297 | GCA_001034405.1 | 615 | 615 | Serratia marcescens | UCI88 | Proteobacteria | KQ089789.1 | III | 2 |
| 1298 | GCA_001537045.1 | 615 | 615 | Serratia marcescens | 2880STDY5682914 | Proteobacteria | FCMH01000002.1 | III | 2 |
| 1299 | GCA_001537345.1 | 615 | 615 | Serratia marcescens | 2880STDY5682872 | Proteobacteria | FCHZ01000003.1 | III | 2 |
| 1300 | GCA_000633715.1 | 1445864 | 615 | Serratia marcescens BIDMC 80 | BIDMC 80 | Proteobacteria | KK214321.1 | III | 2 |
| 1301 | GCA_000163595.1 | 667129 | 618 | Serratia odorifera DSM 4582 | DSM 4582 | Proteobacteria | GG753567.1 | III | 2 |
| 1302 | GCA_000743355.1 | 1524467 | 1524467 | Serratia sp. Ag1 | Ag1 | Proteobacteria | JQEI01000016.1 | III | 2 |
| 1303 | GCA_000743365.1 | 1532556 | 1532556 | Serratia sp. Ag2 | Ag2 | Proteobacteria | JQEJ01000056.1 | III | 2 |
| 1304 | GCA_000203935.1 | 94122 | 94122 | Shewanella sp. ANA-3 | ANA-3 | Proteobacteria | CP000469.1 | III | 2 |
| 1305 | GCA_000262305.1 | 630626 | 563 | Shimwellia blattae DSM 4481 = NBRC 105725 | DSM 4481 | Proteobacteria | CP001560.1 | III | 2 |
| 1306 | GCA_000327265.1 | 630626 | 563 | Shimwellia blattae DSM 4481 = NBRC 105725 | NBRC 105725 | Proteobacteria | BAHA01000013.1 | III | 2 |
| 1307 | GCA_000018545.1 | 394 | 380 | Sinorhizobium fredii NGR234 | NGR234 | Proteobacteria | CP001389.1 | III | 2 |
| 1308 | GCA_000067165.1 | 448385 | 56 | Sorangium cellulosum So ce56 | So ce 56 | Proteobacteria | AM746676.1 | III | 2 |
| 1309 | GCA_000421925.1 | 1346790 | 420233 | Sphingobium chinhatense IP26 | IP26 | Proteobacteria | AUDA01000025.1 | III | 2 |
| 1310 | GCA_001005725.1 | 56193 | 56193 | Sphingobium chungbukense | DJ77 | Proteobacteria | LBIC01000001.1 | III | 2 |
| 1311 | GCA_000382885.1 | 1301087 | 332056 | Sphingobium japonicum BiD32 | BiD32 | Proteobacteria | CAVK010000098.1 | III | 2 |
| 1312 | GCA_000633575.2 | 1450518 | 440518 | Sphingobium lucknowense F2 | F2 | Proteobacteria | JANF02000056.1 | III | 2 |
| 1313 | GCA_001421635.1 | 1735692 | 1735692 | Sphingomonas sp. Leaf25 | Leaf25 | Proteobacteria | LMKU01000030.1 | III | 2 |
| 1314 | GCA_001399755.1 | 381306 | 381306 | Thiohalorhabdus denitrificans | HL 19 | Proteobacteria | LJCP01000010.1 | III | 2 |
| 1315 | GCA_001425205.1 | 1736530 | 1736530 | Variovorax sp. Root411 | Root411 | Proteobacteria | LMDW01000005.1 | III | 2 |
| 1316 | GCA_001010935.1 | 663 | 663 | Vibrio alginolyticus | V2 | Proteobacteria | LCSG01000013.1 | III | 2 |
| 1317 | GCA_000154025.1 | 410291 | 680 | Vibrio campbellii HY01 | HY01 | Proteobacteria | DS179487.1 | III | 2 |
| 1318 | GCA_000785575.1 | 701175 | 701175 | Vibrio caribbeanicus | T14 | Proteobacteria | JRWR01000004.1 | III | 2 |
| 1319 | GCA_000736945.1 | 666 | 666 | Vibrio cholerae | 571-88 | Proteobacteria | JIDO01000026.1 | III | 2 |
| 1320 | GCA_000737005.1 | 666 | 666 | Vibrio cholerae | 234-93 | Proteobacteria | JMBO01000023.1 | III | 2 |
| 1321 | GCA_000279285.1 | 991946 | 666 | Vibrio cholerae HE-45 | HE-45 | Proteobacteria | ALED01000027.1 | III | 2 |
| 1322 | GCA_000348345.2 | 1095649 | 666 | Vibrio cholerae O1 str. EM-1676A | EM-1676A | Proteobacteria | KB662755.1 | III | 2 |
| 1323 | GCA_000152465.2 | 345075 | 666 | Vibrio cholerae V51 | V51 | Proteobacteria | KQ257379.1 | III | 2 |
| 1324 | GCA_000467185.1 | 1219080 | 252583 | Vibrio ezurae NBRC 102218 | NBRC 102218 | Proteobacteria | BATM01000020.1 | III | 2 |
| 1325 | GCA_001262735.1 | 669 | 669 | Vibrio harveyi | VH2 | Proteobacteria | LGYS01000041.1 | III | 2 |
| 1326 | GCA_000786865.1 | 670 | 670 | Vibrio parahaemolyticus | 09.5357 | Proteobacteria | JTGT01000603.1 | III | 2 |
| 1327 | GCA_001270155.1 | 670 | 670 | Vibrio parahaemolyticus | S439-9 | Proteobacteria | LFZA01000109.1 | III | 2 |
| 1328 | GCA_000808535.1 | 379097 | 379097 | Vibrio sinaloensis | AD048 | Proteobacteria | JWLV01000045.1 | III | 2 |
| 1329 | GCA_001597935.1 | 1763885 | 1763885 | Vibrio sp. 2538-88 | 2538-88 | Proteobacteria | LOBR01000072.1 | III | 2 |
| 1330 | GCA_000305755.2 | 992012 | 992012 | Vibrio sp. HENC-03 | HENC-03 | Proteobacteria | JH976122.1 | III | 2 |
| 1331 | GCA_001048635.1 | 1507980 | 1507980 | Vibrio sp. J2-31 | J2-31 | Proteobacteria | CCKO01000056.1 | III | 2 |
| 1332 | GCA_000091465.1 | 575788 | 212663 | Vibrio tasmaniensis LGP32 | LGP32 | Proteobacteria | FM954972.2 | III | 2 |
| 1333 | GCA_001541335.1 | 1194427 | 1194427 | Vibrio toranzoniae | Vb 10.8 | Proteobacteria | LMXU01000013.1 | III | 2 |
| 1334 | GCA_001274855.1 | 170661 | 170661 | Vibrio xuii | DSM 17185 | Proteobacteria | LHPK01000025.1 | III | 2 |
| 1335 | GCA_001131685.1 | 634 | 634 | Yersinia bercovieri | 127/84 | Proteobacteria | CQBU01000002.1 | III | 2 |
| 1336 | GCA_001219605.1 | 630 | 630 | Yersinia enterocolitica | YE04/02 | Proteobacteria | CTKX01000003.1 | III | 2 |
| 1337 | GCA_001220145.1 | 630 | 630 | Yersinia enterocolitica | YE38/03 | Proteobacteria | CTKL01000007.1 | III | 2 |
| 1338 | GCA_001319585.1 | 29484 | 29484 | Yersinia frederiksenii | 38/83 | Proteobacteria | CGCC01000006.1 | III | 2 |
| 1339 | GCA_001142305.1 | 419257 | 419257 | Yersinia massiliensis | 24070 | Proteobacteria | CQBH01000001.1 | III | 2 |
| 1340 | GCA_001319885.1 | 633 | 633 | Yersinia pseudotuberculosis | OK6088 | Proteobacteria | CGGM01000007.1 | III | 2 |
| 1341 | GCA_000737165.1 | 29486 | 29486 | Yersinia ruckeri | 37551 | Proteobacteria | JPF001000008.1 | III | 2 |
| 1342 | GCA_000964565.1 | 29486 | 29486 | Yersinia ruckeri | Big Creek 74 | Proteobacteria | CP011078.1 | III | 2 |
| 1343 | GCA_000239335.1 | 1002368 | 158877 | Yokenella regensburgei ATCC 43003 | ATCC 43003 | Proteobacteria | JH417862.1 | III | 2 |

(Part B)

| | | | | M | | | | Q | | | | U | | | |
|---|---|---|---|---|---|---|---|---|---|---|---|---|---|---|---|
| | | | | DruA | | | | DruB | | | | DruC | | | |
| | J | K | L | protein | N | O | P | protein | R | S | T | protein | V | W | X |
| | DruA | PRT SEQ | DNA SEQ | length | DruB | PRT SEQ | DNA SEQ | length | DruC | PRT SEQ | DNA SEQ | length | DruD | PRT SEQ | DNA SEQ |
| 1 | protein_accession | ID NO: | ID NO: | (aa) | protein_accession | ID NO: | ID NO: | (aa) | protein_accession | ID NO: | ID NO: | (aa) | protein_accession | ID NO: | ID NO: |
| 2 | | | | | CEP95570.1 | 23934 | 27163 | 531 | CEP95571.1 | 23935 | 27164 | 669 | CEP95572.1 | 23936 | 27165 |
| 3 | KUO96130.1 | 23938 | 27167 | 411 | KUO96131.1 | 23939 | 27168 | 468 | KUO96132.1 | 23940 | 27169 | 604 | KUO96133.1 | 23941 | 27170 |
| 4 | | | | | GAC90451.1 | 23943 | 27172 | 504 | GAC90452.1 | 23944 | 27173 | 659 | GAC90453.1 | 23945 | 27174 |
| 5 | KQT61309.1 | 23947 | 27176 | 678 | | | | | | | | | KQT61312.1 | 23948 | 27177 |
| 6 | KQT62611.1 | 23950 | 27179 | 678 | | | | | | | | | KQT62614.1 | 23951 | 27180 |
| 7 | | | | | KMN72587.1 | 23953 | 27182 | 502 | KMN72586.1 | 23954 | 27183 | 662 | KMN72585.1 | 23955 | 27184 |
| 8 | | | | | EJQ91443.1 | 23957 | 27186 | 502 | EJQ91444.1 | 23958 | 27187 | 662 | EJQ91445.1 | 23959 | 27188 |
| 9 | | | | | CRK83009.1 | 23961 | 27190 | 481 | CRK83010.1 | 23962 | 27191 | 640 | CRK83011.1 | 23963 | 27192 |
| 10 | | | | | KEF29420.1 | 23965 | 27194 | 519 | | | | | KEF29418.1 | 23966 | 27195 |
| 11 | | | | | ETO96271.1 | 23968 | 27197 | 513 | | | | | ETO96375.1 | 23969 | 27198 |
| 12 | ALK08980.1 | 23971 | 27200 | 418 | | | | | | | | | ALK08978.1 | 23972 | 27201 |
| 13 | CUU41641.1 | 23974 | 27203 | 418 | | | | | | | | | CUU41639.1 | 23975 | 27204 |
| 14 | KGC93367.1 | 23977 | 27206 | 400 | KGC93228.1 | 23978 | 27207 | 505 | KGC93290.1 | 23979 | 27208 | 854 | KGC93264.1 | 23980 | 27209 |
| 15 | KGV74617.1 | 23982 | 27211 | 405 | | | | | | | | | KGV74230.1 | 23983 | 27212 |
| 16 | EDO89776.1 | 23985 | 27214 | 400 | EDO89775.1 | 23986 | 27215 | 523 | EDO89774.1 | 23987 | 27216 | 858 | | | |
| 17 | AJX60919.1 | 23989 | 27218 | 400 | AJX61068.1 | 23990 | 27219 | 457 | AJX59269.1 | 23991 | 27220 | 858 | AJX59244.1 | 23992 | 27221 |
| 18 | AIP62558.1 | 23994 | 27223 | 400 | AJT48645.1 | 23995 | 27224 | 211 | | | | | | | |
| 19 | KVO65551.1 | 23997 | 27226 | 400 | KVO65550.1 | 23998 | 27227 | 211 | KVO65549.1 | 23999 | 27228 | 841 | | | |
| 20 | KVO87083.1 | 24001 | 27230 | 400 | KVO87084.1 | 24002 | 27231 | 211 | | | | | | | |
| 21 | CDI04329.1 | 24004 | 27233 | 401 | | | | | | | | | CDI04327.1 | 24005 | 27234 |
| 22 | | | | | KPA09471.1 | 24007 | 27236 | 522 | KPA09470.1 | 24008 | 27237 | 594 | KPA09469.1 | 24009 | 27238 |
| 23 | | | | | | | | | KHD06506.1 | 24011 | 27240 | 632 | KHD06507.1 | 24012 | 27241 |
| 24 | KJH65670.1 | 24014 | 27243 | 409 | KJH65671.1 | 24015 | 27244 | 555 | KJH65672.1 | 24016 | 27245 | 623 | KJH65673.1 | 24017 | 27246 |
| 25 | | | | | CDM70052.1 | 24019 | 27248 | 531 | CDM70054.1 | 24020 | 27249 | 299 | CDM70055.1 | 24021 | 27250 |
| 26 | | | | | | | | | KEI16341.1 | 24023 | 27252 | 674 | KEI16342.1 | 24024 | 27253 |
| 27 | | | | | EGB92907.1 | 24026 | 27255 | 478 | EGB92908.1 | 24027 | 27256 | 483 | EGB92909.1 | 24028 | 27257 |
| 28 | | | | | ACL18694.1 | 24030 | 27259 | 479 | | | | | ACL18696.1 | 24031 | 27260 |
| 29 | | | | | ACV64027.1 | 24033 | 27262 | 480 | | | | | ACV64029.1 | 24034 | 27263 |
| 30 | | | | | AMC93642.1 | 24036 | 27265 | 481 | | | | | AMC93640.1 | 24037 | 27266 |
| 31 | KHG78165.1 | 24039 | 27268 | 404 | KHG78164.1 | 24040 | 27269 | 548 | KHG78163.1 | 24041 | 27270 | 627 | KHG78162.1 | 24042 | 27271 |
| 32 | KHI73843.1 | 24044 | 27273 | 404 | KHI73842.1 | 24045 | 27274 | 548 | KHI73841.1 | 24046 | 27275 | 627 | KHI73840.1 | 24047 | 27276 |
| 33 | | | | | | | | | KHI66626.1 | 24049 | 27278 | 398 | KHI66627.1 | 24050 | 27279 |
| 34 | KHI48864.1 | 24052 | 27281 | 404 | KHI48865.1 | 24053 | 27282 | 548 | KHI48866.1 | 24054 | 27283 | 627 | KHI48867.1 | 24055 | 27284 |
| 35 | KHI24189.1 | 24057 | 27286 | 404 | | | | | KHI24188.1 | 24058 | 27287 | 627 | KHI24187.1 | 24059 | 27288 |
| 36 | KMV42884.1 | 24061 | 27290 | 404 | KMV42883.1 | 24062 | 27291 | 548 | KMV42882.1 | 24063 | 27292 | 627 | KMV42881.1 | 24064 | 27293 |
| 37 | KQJ50747.1 | 24066 | 27295 | 404 | KQJ50746.1 | 24067 | 27296 | 548 | KQJ50745.1 | 24068 | 27297 | 627 | KQJ50744.1 | 24069 | 27298 |
| 38 | ALN47010.1 | 24071 | 27300 | 404 | ALN47011.1 | 24072 | 27301 | 548 | ALN47012.1 | 24073 | 27302 | 627 | ALN47013.1 | 24074 | 27303 |
| 39 | KSY14484.1 | 24076 | 27305 | 404 | KSY14483.1 | 24077 | 27306 | 548 | KSY14482.1 | 24078 | 27307 | 627 | KSY14481.1 | 24079 | 27308 |
| 40 | KXR68579.1 | 24081 | 27310 | 404 | KXR68578.1 | 24082 | 27311 | 548 | KXR68577.1 | 24083 | 27312 | 627 | KXR68576.1 | 24084 | 27313 |
| 41 | KIO85517.1 | 24086 | 27315 | 404 | KIO85917.1 | 24087 | 27316 | 548 | KIO85687.1 | 24088 | 27317 | 627 | KIO84333.1 | 24089 | 27318 |
| 42 | EQS48043.1 | 24091 | 27320 | 404 | EQS48044.1 | 24092 | 27321 | 548 | EQS48045.1 | 24093 | 27322 | 627 | EQS48046.1 | 24094 | 27323 |
| 43 | EQO85266.1 | 24096 | 27325 | 404 | EQO85267.1 | 24097 | 27326 | 548 | EQO85268.1 | 24098 | 27327 | 627 | EQO85269.1 | 24099 | 27328 |
| 44 | EQO74905.1 | 24101 | 27330 | 404 | EQO74906.1 | 24102 | 27331 | 548 | EQO74907.1 | 24103 | 27332 | 627 | EQO74908.1 | 24104 | 27333 |
| 45 | EYT06138.1 | 24106 | 27335 | 404 | EYT06139.1 | 24107 | 27336 | 548 | EYT06140.1 | 24108 | 27337 | 627 | EYT06141.1 | 24109 | 27338 |
| 46 | EQV41654.1 | 24111 | 27340 | 404 | EQV41653.1 | 24112 | 27341 | 548 | EQV41652.1 | 24113 | 27342 | 627 | EQV41651.1 | 24114 | 27343 |
| 47 | ELC24068.1 | 24116 | 27345 | 404 | ELC24069.1 | 24117 | 27346 | 548 | ELC24070.1 | 24118 | 27347 | 627 | ELC24071.1 | 24119 | 27348 |
| 48 | ELH39084.1 | 24121 | 27350 | 404 | ELH39085.1 | 24122 | 27351 | 548 | ELH39086.1 | 24123 | 27352 | 627 | ELH39087.1 | 24124 | 27353 |
| 49 | EOV00819.1 | 24126 | 27355 | 404 | EOV00818.1 | 24127 | 27356 | 548 | EOV00817.1 | 24128 | 27357 | 627 | EOV00816.1 | 24129 | 27358 |
| 50 | EOU57734.1 | 24131 | 27360 | 404 | EOU57733.1 | 24132 | 27361 | 548 | EOU57732.1 | 24133 | 27362 | 627 | EOU57731.1 | 24134 | 27363 |
| 51 | ELE47058.1 | 24136 | 27365 | 404 | ELE47059.1 | 24137 | 27366 | 548 | ELE47060.1 | 24138 | 27367 | 627 | ELE47061.1 | 24139 | 27368 |
| 52 | ELE76866.1 | 24141 | 27370 | 404 | ELE76867.1 | 24142 | 27371 | 548 | ELE76868.1 | 24143 | 27372 | 627 | ELE76869.1 | 24144 | 27373 |
| 53 | EU64783.1 | 24146 | 27375 | 404 | ELJ64784.1 | 24147 | 27376 | 548 | ELJ64785.1 | 24148 | 27377 | 627 | ELJ64786.1 | 24149 | 27378 |
| 54 | EOV85617.1 | 24151 | 27380 | 404 | EOV85618.1 | 24152 | 27381 | 548 | EOV85619.1 | 24153 | 27382 | 627 | EOV85620.1 | 24154 | 27383 |
| 55 | ELE86430.1 | 24156 | 27385 | 404 | ELE86431.1 | 24157 | 27386 | 548 | ELE86432.1 | 24158 | 27387 | 627 | ELE86433.1 | 24159 | 27388 |
| 56 | EWY52046.1 | 24161 | 27390 | 404 | EWY52045.1 | 24162 | 27391 | 548 | EWY52044.1 | 24163 | 27392 | 627 | EWY52043.1 | 24164 | 27393 |
| 57 | EFK51863.1 | 24166 | 27395 | 404 | EFK51864.1 | 24167 | 27396 | 251 | EFK51865.1 | 24168 | 27397 | 627 | EFK51866.1 | 24169 | 27398 |
| 58 | | | | | | | | | EGU96601.1 | 24171 | 27400 | 530 | EGU96602.1 | 24172 | 27401 |
| 59 | EMZ39757.1 | 24174 | 27403 | 404 | EMZ39756.1 | 24175 | 27404 | 548 | EMZ39755.1 | 24176 | 27405 | 627 | EMZ39754.1 | 24177 | 27406 |
| 60 | | | | | EGI33548.1 | 24179 | 27408 | 548 | EGI33546.1 | 24180 | 27409 | 403 | EGI33545.1 | 24181 | 27410 |
| 61 | EQX30244.1 | 24183 | 27412 | 404 | EQX30245.1 | 24184 | 27413 | 548 | EQX30246.1 | 24185 | 27414 | 627 | EQX30247.1 | 24186 | 27415 |
| 62 | EQZ12277.1 | 24188 | 27417 | 404 | EQZ12278.1 | 24189 | 27418 | 548 | EQZ12279.1 | 24190 | 27419 | 627 | EQZ12280.1 | 24191 | 27420 |
| 63 | ERA40829.1 | 24193 | 27422 | 404 | ERA40830.1 | 24194 | 27423 | 548 | ERA40831.1 | 24195 | 27424 | 627 | ERA40832.1 | 24196 | 27425 |
| 64 | | | | | CDA97617.1 | 24198 | 27427 | 479 | CDA97616.1 | 24199 | 27428 | 635 | CDA97615.1 | 24200 | 27429 |
| 65 | | | | | KFX30971.1 | 24202 | 27431 | 432 | KFX30970.1 | 24203 | 27432 | 660 | KFX30969.1 | 24204 | 27433 |
| 66 | | | | | KYD25149.1 | 24206 | 27435 | 506 | KYD25148.1 | 24207 | 27436 | 660 | KYD25147.1 | 24208 | 27437 |
| 67 | | | | | KLR73615.1 | 24210 | 27439 | 182 | | | | | KLR73616.1 | 24211 | 27440 |
| 68 | | | | | ADU94762.1 | 24213 | 27442 | 432 | ADU94761.1 | 24214 | 27443 | 660 | ADU94760.1 | 24215 | 27444 |
| 69 | | | | | ACX76983.1 | 24217 | 27446 | 432 | ACX76984.1 | 24218 | 27447 | 660 | ACX76985.1 | 24219 | 27448 |
| 70 | | | | | KFL17490.1 | 24221 | 27450 | 253 | KFL17491.1 | 24222 | 27451 | 660 | KFL17492.1 | 24223 | 27452 |
| 71 | | | | | KFX35838.1 | 24225 | 27454 | 432 | KFX35839.1 | 24226 | 27455 | 660 | KFX35840.1 | 24227 | 27456 |
| 72 | EDQ34548.2 | 24229 | 27458 | 406 | | | | | | | | | EDQ34544.2 | 24230 | 27459 |
| 73 | KRB96408.1 | 24232 | 27461 | 398 | | | | | | | | | KRB96406.1 | 24233 | 27462 |
| 74 | AGK57206.1 | 24235 | 27464 | 419 | | | | | | | | | AGK57208.1 | 24236 | 27465 |
| 75 | KMV96850.1 | 24238 | 27467 | 404 | KMV96849.1 | 24239 | 27468 | 549 | KMV96848.1 | 24240 | 27469 | 627 | KMV96847.1 | 24241 | 27470 |
| 76 | ESN04444.1 | 24243 | 27472 | 406 | | | | | ESN04438.1 | 24244 | 27473 | 903 | ESN04442.1 | 24245 | 27474 |
| 77 | | | | | | | | | ABX40973.1 | 24247 | 27476 | 648 | ABX40974.1 | 24248 | 27477 |
| 78 | | | | | EOS72131.1 | 24250 | 27479 | 479 | EOS72130.1 | 24251 | 27480 | 636 | EOS72129.1 | 24252 | 27481 |
| 79 | | | | | EDM25760.1 | 24254 | 27483 | 513 | EDM25761.1 | 24255 | 27484 | 674 | | | |
| 80 | ESY17003.1 | 24257 | 27486 | 676 | | | | | | | | | ESY17069.1 | 24258 | 27487 |
| 81 | | | | | KUK70368.1 | 24260 | 27489 | 537 | KUK70367.1 | 24261 | 27490 | 831 | KUK70366.1 | 24262 | 27491 |
| 82 | | | | | EHQ36502.1 | 24264 | 27493 | 540 | EHQ36503.1 | 24265 | 27494 | 721 | EHQ36504.1 | 24266 | 27495 |
| 83 | | | | | KKG83063.1 | 24268 | 27497 | 529 | KKG83062.1 | 24269 | 27498 | 719 | KKG83061.1 | 24270 | 27499 |
| 84 | EQM86812.1 | 24272 | 27501 | 662 | | | | | | | | | EQM86809.1 | 24273 | 27502 |
| 85 | AU18572.1 | 24275 | 27504 | 661 | | | | | | | | | ALJ18569.1 | 24276 | 27505 |
| 86 | GAD34403.1 | 24278 | 27507 | 661 | | | | | | | | | GAD34407.1 | 24279 | 27508 |
| 87 | ADJ29888.1 | 24281 | 27510 | 432 | | | | | | | | | ADJ29890.1 | 24282 | 27511 |
| 88 | KMP10704.1 | 24284 | 27513 | 402 | | | | | | | | | KMP10706.1 | 24285 | 27514 |
| 89 | EGY00299.1 | 24287 | 27516 | 677 | | | | | | | | | EGY00302.1 | 24288 | 27517 |
| 90 | | | | | EPY09175.1 | 24290 | 27519 | 473 | EPY09174.1 | 24291 | 27520 | 646 | EPY09173.1 | 24292 | 27521 |
| 91 | | | | | ACX63337.1 | 24294 | 27523 | 545 | ACX63338.1 | 24295 | 27524 | 656 | ACX63339.1 | 24296 | 27525 |
| 92 | KHN90278.1 | 24298 | 27527 | 404 | KHN90279.1 | 24299 | 27528 | 537 | KHN90280.1 | 24300 | 27529 | 627 | KHN90281.1 | 24301 | 27530 |
| 93 | KGA32599.1 | 24303 | 27532 | 406 | | | | | | | | | KGA32597.1 | 24304 | 27533 |
| 94 | | | | | AJQ25677.1 | 24306 | 27535 | 477 | AJQ25678.1 | 24307 | 27536 | 527 | AJQ25679.1 | 24308 | 27537 |
| 95 | | | | | CUH95392.1 | 24310 | 27539 | 476 | CUH95393.1 | 24311 | 27540 | 527 | CUH95394.1 | 24312 | 27541 |
| 96 | KSF97359.1 | 24314 | 27543 | 406 | KSF97360.1 | 24315 | 27544 | 425 | KSF97361.1 | 24316 | 27545 | 630 | | | |
| 97 | | | | | | | | | KSL25396.1 | 24318 | 27547 | 116 | KSL25395.1 | 24319 | 27548 |
| 98 | KSO15708.1 | 24321 | 27550 | 406 | KSO15707.1 | 24322 | 27551 | 425 | KSO15706.1 | 24323 | 27552 | 630 | | | |
| 99 | BAR67377.1 | 24325 | 27554 | 409 | BAR67378.1 | 24326 | 27555 | 556 | BAR67379.1 | 24327 | 27556 | 625 | BAR67380.1 | 24328 | 27557 |
| 100 | KAJ21868.1 | 24330 | 27559 | 343 | | | | | KAJ21867.1 | 24331 | 27560 | 630 | KAJ21866.1 | 24332 | 27561 |
| 101 | AGI81181.1 | 24334 | 27563 | 409 | | | | | AGI81182.1 | 24335 | 27564 | 464 | | | |
| 102 | ERY34412.1 | 24337 | 27566 | 411 | ERY34411.1 | 24338 | 27567 | 555 | ERY34410.1 | 24339 | 27568 | 630 | ERY34409.1 | 24340 | 27569 |
| 103 | EZP23709.1 | 24342 | 27571 | 409 | EZP23710.1 | 24343 | 27572 | 476 | EZP23711.1 | 24344 | 27573 | 625 | EZP23712.1 | 24345 | 27574 |
| 104 | ERV69455.1 | 24347 | 27576 | 406 | ERV69456.1 | 24348 | 27577 | 548 | ERV69457.1 | 24349 | 27578 | 611 | ERV69458.1 | 24350 | 27579 |
| 105 | EQM83903.1 | 24352 | 27581 | 268 | EQM83904.1 | 24353 | 27582 | 425 | EQM83905.1 | 24354 | 27583 | 630 | | | |
| 106 | AJP52372.1 | 24356 | 27585 | 409 | AJP52373.1 | 24357 | 27586 | 555 | AJP52374.1 | 24358 | 27587 | 629 | AJP52375.1 | 24359 | 27588 |
| 107 | EST14444.1 | 24361 | 27590 | 409 | EST14388.1 | 24362 | 27591 | 555 | EST14448.1 | 24363 | 27592 | 629 | EST14478.1 | 24364 | 27593 |
| 108 | ENY74498.1 | 24366 | 27595 | 411 | ENY74499.1 | 24367 | 27596 | 479 | ENY74500.1 | 24368 | 27597 | 630 | ENY74501.1 | 24369 | 27598 |
| 109 | KWR75280.1 | 24371 | 27600 | 409 | | | | | | | | | KWR75281.1 | 24372 | 27601 |
| 110 | KFE46436.1 | 24374 | 27603 | 409 | KFE46437.1 | 24375 | 27604 | 554 | KFE46438.1 | 24376 | 27605 | 630 | KFE46439.1 | 24377 | 27606 |
| 111 | AMP72541.1 | 24379 | 27608 | 413 | AMP72542.1 | 24380 | 27609 | 477 | AMP72543.1 | 24381 | 27610 | 623 | AMP72544.1 | 24382 | 27611 |
| 112 | KQQ11279.1 | 24384 | 27613 | 652 | | | | | | | | | KQQ11277.1 | 24385 | 27614 |
| 113 | KQS82596.1 | 24387 | 27616 | 679 | | | | | | | | | KQS82593.1 | 24388 | 27617 |
| 114 | KQS78959.1 | 24390 | 27619 | 679 | | | | | | | | | KQS78956.1 | 24391 | 27620 |
| 115 | | | | | | | | | AKE89966.1 | 24393 | 27622 | 712 | AKE89965.1 | 24394 | 27623 |
| 116 | | | | | | | | | EME23883.1 | 24396 | 27625 | 608 | EME23884.1 | 24397 | 27626 |
| 117 | | | | | CDZ88170.1 | 24399 | 27628 | 515 | CDZ88169.1 | 24400 | 27629 | 713 | CDZ88168.1 | 24401 | 27630 |
| 118 | ETI62660.1 | 24403 | 27632 | 661 | | | | | | | | | ETI62671.1 | 24404 | 27633 |
| 119 | EFL24973.1 | 24406 | 27635 | 677 | | | | | | | | | EFL24976.1 | 24407 | 27636 |
| 120 | | | | | AGA58895.1 | 24409 | 27638 | 486 | | | | | AGA58893.1 | 24410 | 27639 |
| 121 | EEY51338.1 | 24412 | 27641 | 397 | EEY51339.1 | 24413 | 27642 | 556 | EEY51340.1 | 24414 | 27643 | 629 | EEY51341.1 | 24415 | 27644 |
| 122 | AIL69849.1 | 24417 | 27646 | 397 | AIL69850.1 | 24418 | 27647 | 556 | AIL69851.1 | 24419 | 27648 | 629 | AIL69852.1 | 24420 | 27649 |
| 123 | CNL41774.1 | 24422 | 27651 | 404 | CNL41795.1 | 24423 | 27652 | 465 | CNL41824.1 | 24424 | 27653 | 627 | CNL41849.1 | 24425 | 27654 |
| 124 | | | | | | | | | | | | | | | |
| 125 | | | | | | | | | | | | | | | |
| 126 | | | | | | | | | | | | | | | |
| 127 | | | | | | | | | | | | | | | |
| 128 | | | | | | | | | | | | | | | |
| 129 | | | | | | | | | | | | | | | |
| 130 | | | | | | | | | | | | | | | |
| 131 | | | | | | | | | | | | | | | |
| 132 | | | | | | | | | | | | | | | |
| 133 | | | | | | | | | | | | | | | |
| 134 | | | | | | | | | | | | | | | |
| 135 | | | | | | | | | | | | | | | |
| 136 | | | | | | | | | | | | | | | |
| 137 | | | | | | | | | | | | | | | |
| 138 | | | | | | | | | | | | | | | |
| 139 | | | | | | | | | | | | | | | |
| 140 | | | | | | | | | | | | | | | |
| 141 | | | | | | | | | | | | | | | |
| 142 | | | | | | | | | | | | | | | |
| 143 | | | | | | | | | | | | | | | |
| 144 | | | | | | | | | | | | | | | |
| 145 | | | | | | | | | | | | | | | |
| 146 | | | | | | | | | | | | | | | |
| 147 | | | | | | | | | | | | | | | |
| 148 | | | | | | | | | | | | | | | |
| 149 | | | | | | | | | | | | | | | |
| 150 | | | | | | | | | | | | | | | |
| 151 | | | | | | | | | | | | | | | |
| 152 | | | | | | | | | | | | | | | |
| 153 | | | | | | | | | | | | | | | |
| 154 | | | | | | | | | | | | | | | |
| 155 | | | | | | | | | | | | | | | |
| 156 | | | | | | | | | | | | | | | |
| 157 | | | | | | | | | | | | | | | |
| 158 | | | | | | | | | | | | | | | |
| 159 | | | | | | | | | | | | | | | |
| 160 | | | | | | | | | | | | | | | |
| 161 | | | | | | | | | | | | | | | |
| 162 | | | | | | | | | | | | | | | |
| 163 | | | | | | | | | | | | | | | |
| 164 | | | | | | | | | | | | | | | |
| 165 | | | | | | | | | | | | | | | |
| 166 | | | | | | | | | | | | | | | |
| 167 | | | | | | | | | | | | | | | |
| 168 | | | | | | | | | | | | | | | |
| 169 | | | | | | | | | | | | | | | |
| 170 | | | | | | | | | | | | | | | |
| 171 | | | | | | | | | | | | | | | |
| 172 | | | | | | | | | | | | | | | |
| 173 | | | | | | | | | | | | | | | |
| 174 | | | | | | | | | | | | | | | |
| 175 | | | | | | | | | | | | | | | |
| 176 | | | | | | | | | | | | | | | |
| 177 | | | | | | | | | | | | | | | |
| 178 | | | | | | | | | | | | | | | |
| 179 | | | | | | | | | | | | | | | |
| 180 | | | | | | | | | | | | | | | |
| 181 | | | | | | | | | | | | | | | |
| 182 | | | | | | | | | | | | | | | |
| 183 | | | | | | | | | | | | | | | |
| 184 | | | | | | | | | | | | | | | |
| 185 | | | | | | | | | | | | | | | |
| 186 | | | | | | | | | | | | | | | |
| 187 | | | | | | | | | | | | | | | |
| 188 | | | | | | | | | | | | | | | |
| 189 | | | | | | | | | | | | | | | |
| 190 | | | | | | | | | | | | | | | |
| 191 | | | | | | | | | | | | | | | |
| 192 | | | | | | | | | | | | | | | |
| 193 | | | | | | | | | | | | | | | |
| 194 | | | | | | | | | | | | | | | |
| 195 | | | | | | | | | | | | | | | |
| 196 | | | | | | | | | | | | | | | |
| 197 | | | | | | | | | | | | | | | |
| 198 | | | | | | | | | | | | | | | |
| 199 | | | | | | | | | | | | | | | |
| 200 | | | | | | | | | | | | | | | |
| 201 | | | | | | | | | | | | | | | |
| 202 | | | | | | | | | | | | | | | |
| 203 | | | | | | | | | | | | | | | |
| 204 | | | | | | | | | | | | | | | |
| 205 | | | | | | | | | | | | | | | |
| 206 | | | | | | | | | | | | | | | |
| 207 | | | | | | | | | | | | | | | |
| 208 | | | | | | | | | | | | | | | |
| 209 | | | | | | | | | | | | | | | |
| 210 | | | | | | | | | | | | | | | |
| 211 | | | | | | | | | | | | | | | |
| 212 | | | | | | | | | | | | | | | |
| 213 | | | | | | | | | | | | | | | |
| 214 | | | | | | | | | | | | | | | |
| 215 | | | | | | | | | | | | | | | |
| 216 | | | | | | | | | | | | | | | |
| 217 | | | | | | | | | | | | | | | |
| 218 | | | | | | | | | | | | | | | |
| 219 | | | | | | | | | | | | | | | |
| 220 | | | | | | | | | | | | | | | |
| 221 | | | | | | | | | | | | | | | |
| 222 | | | | | | | | | | | | | | | |
| 223 | | | | | | | | | | | | | | | |
| 224 | | | | | | | | | | | | | | | |
| 225 | | | | | | | | | | | | | | | |
| 226 | | | | | | | | | | | | | | | |
| 227 | | | | | | | | | | | | | | | |
| 228 | | | | | | | | | | | | | | | |
| 229 | | | | | | | | | | | | | | | |
| 230 | | | | | | | | | | | | | | | |
| 231 | | | | | | | | | | | | | | | |
| 232 | | | | | | | | | | | | | | | |
| 233 | | | | | | | | | | | | | | | |
| 234 | | | | | | | | | | | | | | | |
| 235 | | | | | | | | | | | | | | | |
| 236 | | | | | | | | | | | | | | | |
| 237 | | | | | | | | | | | | | | | |
| 238 | | | | | | | | | | | | | | | |
| 239 | | | | | | | | | | | | | | | |
| 240 | | | | | | | | | | | | | | | |
| 241 | | | | | | | | | | | | | | | |
| 242 | | | | | | | | | | | | | | | |
| 243 | | | | | | | | | | | | | | | |
| 244 | | | | | | | | | | | | | | | |
| 245 | | | | | | | | | | | | | | | |
| 246 | | | | | | | | | | | | | | | |
| 247 | | | | | | | | | | | | | | | |
| 248 | | | | | | | | | | | | | | | |
| 249 | | | | | | | | | | | | | | | |
| 250 | | | | | | | | | | | | | | | |
| 251 | | | | | | | | | | | | | | | |
| 252 | | | | | | | | | | | | | | | |
| 253 | | | | | | | | | | | | | | | |
| 254 | | | | | | | | | | | | | | | |
| 255 | | | | | | | | | | | | | | | |
| 256 | | | | | | | | | | | | | | | |
| 257 | | | | | | | | | | | | | | | |
| 258 | | | | | | | | | | | | | | | |
| 259 | | | | | | | | | | | | | | | |
| 260 | | | | | | | | | | | | | | | |
| 261 | | | | | | | | | | | | | | | |
| 262 | | | | | | | | | | | | | | | |
| 263 | | | | | | | | | | | | | | | |
| 264 | | | | | | | | | | | | | | | |
| 265 | | | | | | | | | | | | | | | |
| 266 | | | | | | | | | | | | | | | |
| 267 | | | | | | | | | | | | | | | |
| 268 | | | | | | | | | | | | | | | |
| 269 | | | | | | | | | | | | | | | |
| 270 | | | | | | | | | | | | | | | |
| 271 | | | | | | | | | | | | | | | |
| 272 | | | | | | | | | | | | | | | |
| 273 | | | | | | | | | | | | | | | |
| 274 | | | | | | | | | | | | | | | |
| 275 | | | | | | | | | | | | | | | |
| 276 | | | | | | | | | | | | | | | |
| 277 | | | | | | | | | | | | | | | |
| 278 | | | | | | | | | | | | | | | |
| 279 | | | | | | | | | | | | | | | |
| 280 | | | | | | | | | | | | | | | |
| 281 | | | | | | | | | | | | | | | |
| 282 | | | | | | | | | | | | | | | |
| 283 | | | | | | | | | | | | | | | |
| 284 | | | | | | | | | | | | | | | |
| 285 | | | | | | | | | | | | | | | |
| 286 | | | | | | | | | | | | | | | |
| 287 | | | | | | | | | | | | | | | |
| 288 | | | | | | | | | | | | | | | |
| 289 | | | | | | | | | | | | | | | |
| 290 | | | | | | | | | | | | | | | |
| 291 | | | | | | | | | | | | | | | |
| 292 | | | | | | | | | | | | | | | |
| 293 | | | | | | | | | | | | | | | |
| 294 | | | | | | | | | | | | | | | |
| 295 | | | | | | | | | | | | | | | |
| 296 | | | | | | | | | | | | | | | |
| 297 | | | | | | | | | | | | | | | |
| 298 | | | | | | | | | | | | | | | |
| 299 | | | | | | | | | | | | | | | |
| 300 | | | | | | | | | | | | | | | |
| 301 | | | | | | | | | | | | | | | |
| 302 | | | | | | | | | | | | | | | |
| 303 | | | | | | | | | | | | | | | |
| 304 | | | | | | | | | | | | | | | |
| 305 | | | | | | | | | | | | | | | |
| 306 | | | | | | | | | | | | | | | |
| 307 | | | | | | | | | | | | | | | |
| 308 | | | | | | | | | | | | | | | |
| 309 | | | | | | | | | | | | | | | |
| 310 | | | | | | | | | | | | | | | |
| 311 | | | | | | | | | | | | | | | |
| 312 | | | | | | | | | | | | | | | |
| 313 | | | | | | | | | | | | | | | |
| 314 | | | | | | | | | | | | | | | |
| 315 | | | | | | | | | | | | | | | |
| 316 | | | | | | | | | | | | | | | |
| 317 | | | | | | | | | | | | | | | |
| 318 | | | | | | | | | | | | | | | |
| 319 | | | | | | | | | | | | | | | |
| 320 | | | | | | | | | | | | | | | |
| 321 | | | | | | | | | | | | | | | |
| 322 | | | | | | | | | | | | | | | |
| 323 | | | | | | | | | | | | | | | |
| 324 | | | | | | | | | | | | | | | |
| 325 | | | | | | | | | | | | | | | |
| 326 | | | | | | | | | | | | | | | |
| 327 | | | | | | | | | | | | | | | |
| 328 | | | | | | | | | | | | | | | |
| 329 | | | | | | | | | | | | | | | |
| 330 | | | | | | | | | | | | | | | |
| 331 | | | | | | | | | | | | | | | |
| 332 | | | | | | | | | | | | | | | |
| 333 | | | | | | | | | | | | | | | |
| 334 | | | | | | | | | | | | | | | |
| 335 | | | | | | | | | | | | | | | |
| 336 | | | | | | | | | | | | | | | |
| 337 | | | | | | | | | | | | | | | |
| 338 | | | | | | | | | | | | | | | |
| 339 | | | | | | | | | | | | | | | |
| 340 | | | | | | | | | | | | | | | |
| 341 | | | | | | | | | | | | | | | |
| 342 | | | | | | | | | | | | | | | |
| 343 | | | | | | | | | | | | | | | |
| 344 | | | | | | | | | | | | | | | |
| 345 | | | | | | | | | | | | | | | |
| 346 | | | | | | | | | | | | | | | |
| 347 | | | | | | | | | | | | | | | |
| 348 | | | | | | | | | | | | | | | |
| 349 | | | | | | | | | | | | | | | |
| 350 | | | | | | | | | | | | | | | |
| 351 | | | | | | | | | | | | | | | |
| 352 | | | | | | | | | | | | | | | |
| 353 | | | | | | | | | | | | | | | |
| 354 | | | | | | | | | | | | | | | |
| 355 | | | | | | | | | | | | | | | |
| 356 | | | | | | | | | | | | | | | |
| 357 | | | | | | | | | | | | | | | |
| 358 | | | | | | | | | | | | | | | |
| 359 | | | | | | | | | | | | | | | |
| 360 | | | | | | | | | | | | | | | |
| 361 | | | | | | | | | | | | | | | |
| 362 | | | | | | | | | | | | | | | |
| 363 | | | | | | | | | | | | | | | |
| 364 | | | | | | | | | | | | | | | |
| 365 | | | | | | | | | | | | | | | |
| 366 | | | | | | | | | | | | | | | |
| 367 | | | | | | | | | | | | | | | |
| 368 | | | | | | | | | | | | | | | |
| 369 | | | | | | | | | | | | | | | |
| 370 | | | | | | | | | | | | | | | |
| 371 | | | | | | | | | | | | | | | |
| 372 | | | | | | | | | | | | | | | |
| 373 | | | | | | | | | | | | | | | |
| 374 | | | | | | | | | | | | | | | |
| 375 | | | | | | | | | | | | | | | |
| 376 | | | | | | | | | | | | | | | |
| 377 | | | | | | | | | | | | | | | |
| 378 | | | | | | | | | | | | | | | |
| 379 | | | | | | | | | | | | | | | |
| 380 | | | | | | | | | | | | | | | |
| 381 | | | | | | | | | | | | | | | |
| 382 | | | | | | | | | | | | | | | |
| 383 | | | | | | | | | | | | | | | |
| 384 | | | | | | | | | | | | | | | |
| 385 | | | | | | | | | | | | | | | |
| 386 | | | | | | | | | | | | | | | |
| 387 | | | | | | | | | | | | | | | |
| 388 | | | | | | | | | | | | | | | |
| 389 | | | | | | | | | | | | | | | |
| 390 | | | | | | | | | | | | | | | |
| 391 | | | | | | | | | | | | | | | |
| 392 | | | | | | | | | | | | | | | |
| 393 | | | | | | | | | | | | | | | |
| 394 | | | | | | | | | | | | | | | |
| 395 | | | | | | | | | | | | | | | |
| 396 | | | | | | | | | | | | | | | |
| 397 | | | | | | | | | | | | | | | |
| 398 | | | | | | | | | | | | | | | |
| 399 | | | | | | | | | | | | | | | |
| 400 | | | | | | | | | | | | | | | |
| 401 | | | | | | | | | | | | | | | |
| 402 | | | | | | | | | | | | | | | |
| 403 | | | | | | | | | | | | | | | |
| 404 | | | | | | | | | | | | | | | |
| 405 | | | | | | | | | | | | | | | |
| 406 | | | | | | | | | | | | | | | |
| 407 | | | | | | | | | | | | | | | |
| 408 | | | | | | | | | | | | | | | |
| 409 | | | | | | | | | | | | | | | |
| 410 | | | | | | | | | | | | | | | |
| 411 | | | | | | | | | | | | | | | |
| 412 | | | | | | | | | | | | | | | |
| 413 | | | | | | | | | | | | | | | |
| 414 | | | | | | | | | | | | | | | |
| 415 | | | | | | | | | | | | | | | |
| 416 | | | | | | | | | | | | | | | |
| 417 | | | | | | | | | | | | | | | |
| 418 | | | | | | | | | | | | | | | |
| 419 | | | | | | | | | | | | | | | |
| 420 | | | | | | | | | | | | | | | |
| 421 | | | | | | | | | | | | | | | |
| 422 | | | | | | | | | | | | | | | |
| 423 | | | | | | | | | | | | | | | |
| 424 | | | | | | | | | | | | | | | |
| 425 | | | | | | | | | | | | | | | |
| 426 | | | | | | | | | | | | | | | |
| 427 | | | | | | | | | | | | | | | |
| 428 | | | | | | | | | | | | | | | |
| 429 | | | | | | | | | | | | | | | |
| 430 | | | | | | | | | | | | | | | |
| 431 | | | | | | | | | | | | | | | |
| 432 | | | | | | | | | | | | | | | |
| 433 | | | | | | | | | | | | | | | |
| 434 | | | | | | | | | | | | | | | |
| 435 | | | | | | | | | | | | | | | |
| 436 | | | | | | | | | | | | | | | |
| 437 | | | | | | | | | | | | | | | |
| 438 | | | | | | | | | | | | | | | |
| 439 | | | | | | | | | | | | | | | |
| 440 | | | | | | | | | | | | | | | |
| 441 | | | | | | | | | | | | | | | |
| 442 | | | | | | | | | | | | | | | |
| 443 | | | | | | | | | | | | | | | |
| 444 | | | | | | | | | | | | | | | |
| 445 | | | | | | | | | | | | | | | |
| 446 | | | | | | | | | | | | | | | |
| 447 | | | | | | | | | | | | | | | |
| 448 | | | | | | | | | | | | | | | |
| 449 | | | | | | | | | | | | | | | |
| 450 | | | | | | | | | | | | | | | |
| 451 | | | | | | | | | | | | | | | |
| 452 | | | | | | | | | | | | | | | |
| 453 | | | | | | | | | | | | | | | |
| 454 | | | | | | | | | | | | | | | |
| 455 | | | | | | | | | | | | | | | |
| 456 | | | | | | | | | | | | | | | |
| 457 | | | | | | | | | | | | | | | |
| 458 | | | | | | | | | | | | | | | |
| 459 | | | | | | | | | | | | | | | |
| 460 | | | | | | | | | | | | | | | |
| 461 | | | | | | | | | | | | | | | |
| 462 | | | | | | | | | | | | | | | |
| 463 | | | | | | | | | | | | | | | |
| 464 | | | | | | | | | | | | | | | |
| 465 | | | | | | | | | | | | | | | |
| 466 | | | | | | | | | | | | | | | |
| 467 | | | | | | | | | | | | | | | |
| 468 | | | | | | | | | | | | | | | |
| 469 | | | | | | | | | | | | | | | |
| 470 | | | | | | | | | | | | | | | |
| 471 | | | | | | | | | | | | | | | |
| 472 | | | | | | | | | | | | | | | |
| 473 | | | | | | | | | | | | | | | |
| 474 | | | | | | | | | | | | | | | |
| 475 | | | | | | | | | | | | | | | |
| 476 | | | | | | | | | | | | | | | |
| 477 | | | | | | | | | | | | | | | |
| 478 | | | | | | | | | | | | | | | |
| 479 | | | | | | | | | | | | | | | |
| 480 | | | | | | | | | | | | | | | |
| 481 | | | | | | | | | | | | | | | |
| 482 | | | | | | | | | | | | | | | |
| 483 | | | | | | | | | | | | | | | |
| 484 | | | | | | | | | | | | | | | |
| 485 | | | | | | | | | | | | | | | |
| 486 | | | | | | | | | | | | | | | |
| 487 | | | | | | | | | | | | | | | |
| 488 | | | | | | | | | | | | | | | |
| 489 | | | | | | | | | | | | | | | |
| 490 | | | | | | | | | | | | | | | |
| 491 | | | | | | | | | | | | | | | |
| 492 | | | | | | | | | | | | | | | |
| 493 | | | | | | | | | | | | | | | |
| 494 | | | | | | | | | | | | | | | |
| 495 | | | | | | | | | | | | | | | |
| 496 | | | | | | | | | | | | | | | |
| 497 | | | | | | | | | | | | | | | |
| 498 | | | | | | | | | | | | | | | |
| 499 | | | | | | | | | | | | | | | |
| 500 | | | | | | | | | | | | | | | |
| 501 | | | | | | | | | | | | | | | |
| 502 | | | | | | | | | | | | | | | |
| 503 | | | | | | | | | | | | | | | |
| 504 | | | | | | | | | | | | | | | |
| 505 | | | | | | | | | | | | | | | |
| 506 | | | | | | | | | | | | | | | |
| 507 | | | | | | | | | | | | | | | |
| 508 | | | | | | | | | | | | | | | |
| 509 | | | | | | | | | | | | | | | |
| 510 | | | | | | | | | | | | | | | |
| 511 | | | | | | | | | | | | | | | |
| 512 | | | | | | | | | | | | | | | |
| 513 | | | | | | | | | | | | | | | |
| 514 | | | | | | | | | | | | | | | |
| 515 | | | | | | | | | | | | | | | |
| 516 | | | | | | | | | | | | | | | |
| 517 | | | | | | | | | | | | | | | |
| 518 | | | | | | | | | | | | | | | |
| 519 | | | | | | | | | | | | | | | |
| 520 | | | | | | | | | | | | | | | |
| 521 | | | | | | | | | | | | | | | |
| 522 | | | | | | | | | | | | | | | |
| 523 | | | | | | | | | | | | | | | |
| 524 | | | | | | | | | | | | | | | |
| 525 | | | | | | | | | | | | | | | |
| 526 | | | | | | | | | | | | | | | |
| 527 | | | | | | | | | | | | | | | |
| 528 | | | | | | | | | | | | | | | |
| 529 | | | | | | | | | | | | | | | |
| 530 | | | | | | | | | | | | | | | |
| 531 | | | | | | | | | | | | | | | |
| 532 | | | | | | | | | | | | | | | |
| 533 | | | | | | | | | | | | | | | |
| 534 | | | | | | | | | | | | | | | |
| 535 | | | | | | | | | | | | | | | |
| 536 | | | | | | | | | | | | | | | |
| 537 | | | | | | | | | | | | | | | |
| 538 | | | | | | | | | | | | | | | |
| 539 | | | | | | | | | | | | | | | |
| 540 | | | | | | | | | | | | | | | |
| 541 | | | | | | | | | | | | | | | |
| 542 | | | | | | | | | | | | | | | |
| 543 | | | | | | | | | | | | | | | |
| 544 | | | | | | | | | | | | | | | |
| 545 | | | | | | | | | | | | | | | |
| 546 | | | | | | | | | | | | | | | |
| 547 | | | | | | | | | | | | | | | |
| 548 | | | | | | | | | | | | | | | |
| 549 | | | | | | | | | | | | | | | |
| 550 | | | | | | | | | | | | | | | |
| 551 | | | | | | | | | | | | | | | |
| 552 | | | | | | | | | | | | | | | |
| 553 | | | | | | | | | | | | | | | |
| 554 | | | | | | | | | | | | | | | |
| 555 | | | | | | | | | | | | | | | |
| 556 | | | | | | | | | | | | | | | |
| 557 | | | | | | | | | | | | | | | |
| 558 | | | | | | | | | | | | | | | |
| 559 | | | | | | | | | | | | | | | |
| 560 | | | | | | | | | | | | | | | |
| 561 | | | | | | | | | | | | | | | |
| 562 | | | | | | | | | | | | | | | |
| 563 | | | | | | | | | | | | | | | |
| 564 | | | | | | | | | | | | | | | |
| 565 | | | | | | | | | | | | | | | |
| 566 | | | | | | | | | | | | | | | |
| 567 | | | | | | | | | | | | | | | |
| 568 | | | | | | | | | | | | | | | |
| 569 | | | | | | | | | | | | | | | |
| 570 | | | | | | | | | | | | | | | |
| 571 | | | | | | | | | | | | | | | |
| 572 | | | | | | | | | | | | | | | |
| 573 | | | | | | | | | | | | | | | |
| 574 | | | | | | | | | | | | | | | |
| 575 | | | | | | | | | | | | | | | |
| 576 | | | | | | | | | | | | | | | |
| 577 | | | | | | | | | | | | | | | |
| 578 | | | | | | | | | | | | | | | |
| 579 | | | | | | | | | | | | | | | |
| 580 | | | | | | | | | | | | | | | |
| 581 | | | | | | | | | | | | | | | |
| 582 | | | | | | | | | | | | | | | |
| 583 | | | | | | | | | | | | | | | |
| 584 | | | | | | | | | | | | | | | |
| 585 | | | | | | | | | | | | | | | |
| 586 | | | | | | | | | | | | | | | |
| 587 | | | | | | | | | | | | | | | |
| 588 | | | | | | | | | | | | | | | |
| 589 | | | | | | | | | | | | | | | |
| 590 | | | | | | | | | | | | | | | |
| 591 | | | | | | | | | | | | | | | |
| 592 | | | | | | | | | | | | | | | |
| 593 | | | | | | | | | | | | | | | |
| 594 | | | | | | | | | | | | | | | |
| 595 | | | | | | | | | | | | | | | |
| 596 | | | | | | | | | | | | | | | |
| 597 | | | | | | | | | | | | | | | |
| 598 | | | | | | | | | | | | | | | |
| 599 | | | | | | | | | | | | | | | |
| 600 | | | | | | | | | | | | | | | |
| 601 | | | | | | | | | | | | | | | |
| 602 | | | | | | | | | | | | | | | |
| 603 | | | | | | | | | | | | | | | |
| 604 | | | | | | | | | | | | | | | |
| 605 | | | | | | | | | | | | | | | |
| 606 | | | | | | | | | | | | | | | |
| 607 | | | | | | | | | | | | | | | |
| 608 | | | | | | | | | | | | | | | |
| 609 | | | | | | | | | | | | | | | |
| 610 | | | | | | | | | | | | | | | |
| 611 | | | | | | | | | | | | | | | |
| 612 | | | | | | | | | | | | | | | |
| 613 | | | | | | | | | | | | | | | |
| 614 | | | | | | | | | | | | | | | |
| 615 | | | | | | | | | | | | | | | |
| 616 | | | | | | | | | | | | | | | |
| 617 | | | | | | | | | | | | | | | |
| 618 | | | | | | | | | | | | | | | |
| 619 | | | | | | | | | | | | | | | |
| 620 | | | | | | | | | | | | | | | |
| 621 | | | | | | | | | | | | | | | |
| 622 | | | | | | | | | | | | | | | |
| 623 | | | | | | | | | | | | | | | |
| 624 | | | | | | | | | | | | | | | |
| 625 | | | | | | | | | | | | | | | |
| 626 | | | | | | | | | | | | | | | |
| 627 | | | | | | | | | | | | | | | |
| 628 | | | | | | | | | | | | | | | |
| 629 | | | | | | | | | | | | | | | |
| 630 | | | | | | | | | | | | | | | |
| 631 | | | | | | | | | | | | | | | |
| 632 | | | | | | | | | | | | | | | |
| 633 | | | | | | | | | | | | | | | |
| 634 | | | | | | | | | | | | | | | |
| 635 | | | | | | | | | | | | | | | |
| 636 | | | | | | | | | | | | | | | |
| 637 | | | | | | | | | | | | | | | |
| 638 | | | | | | | | | | | | | | | |
| 639 | | | | | | | | | | | | | | | |
| 640 | | | | | | | | | | | | | | | |
| 641 | | | | | | | | | | | | | | | |
| 642 | | | | | | | | | | | | | | | |
| 643 | | | | | | | | | | | | | | | |
| 644 | | | | | | | | | | | | | | | |
| 645 | | | | | | | | | | | | | | | |
| 646 | | | | | | | | | | | | | | | |
| 647 | | | | | | | | | | | | | | | |
| 648 | | | | | | | | | | | | | | | |
| 649 | | | | | | | | | | | | | | | |
| 650 | | | | | | | | | | | | | | | |
| 651 | | | | | | | | | | | | | | | |
| 652 | | | | | | | | | | | | | | | |
| 653 | | | | | | | | | | | | | | | |
| 654 | | | | | | | | | | | | | | | |
| 655 | | | | | | | | | | | | | | | |
| 656 | | | | | | | | | | | | | | | |
| 657 | | | | | | | | | | | | | | | |
| 658 | | | | | | | | | | | | | | | |
| 659 | | | | | | | | | | | | | | | |
| 660 | | | | | | | | | | | | | | | |
| 661 | | | | | | | | | | | | | | | |
| 662 | | | | | | | | | | | | | | | |
| 663 | | | | | | | | | | | | | | | |
| 664 | | | | | | | | | | | | | | | |
| 665 | | | | | | | | | | | | | | | |
| 666 | | | | | | | | | | | | | | | |
| 667 | | | | | | | | | | | | | | | |
| 668 | | | | | | | | | | | | | | | |
| 669 | | | | | | | | | | | | | | | |
| 670 | | | | | | | | | | | | | | | |
| 671 | | | | | | | | | | | | | | | |
| 672 | | | | | | | | | | | | | | | |
| 673 | | | | | | | | | | | | | | | |
| 674 | | | | | | | | | | | | | | | |
| 675 | | | | | | | | | | | | | | | |
| 676 | | | | | | | | | | | | | | | |
| 677 | | | | | | | | | | | | | | | |
| 678 | | | | | | | | | | | | | | | |
| 679 | | | | | | | | | | | | | | | |
| 680 | | | | | | | | | | | | | | | |
| 681 | | | | | | | | | | | | | | | |
| 682 | | | | | | | | | | | | | | | |
| 683 | | | | | | | | | | | | | | | |
| 684 | | | | | | | | | | | | | | | |
| 685 | | | | | | | | | | | | | | | |
| 686 | | | | | | | | | | | | | | | |
| 687 | | | | | | | | | | | | | | | |
| 688 | | | | | | | | | | | | | | | |
| 689 | | | | | | | | | | | | | | | |
| 690 | | | | | | | | | | | | | | | |
| 691 | | | | | | | | | | | | | | | |
| 692 | | | | | | | | | | | | | | | |
| 693 | | | | | | | | | | | | | | | |
| 694 | | | | | | | | | | | | | | | |
| 695 | | | | | | | | | | | | | | | |
| 696 | | | | | | | | | | | | | | | |
| 697 | | | | | | | | | | | | | | | |
| 698 | | | | | | | | | | | | | | | |
| 699 | | | | | | | | | | | | | | | |
| 700 | | | | | | | | | | | | | | | |
| 701 | | | | | | | | | | | | | | | |
| 702 | | | | | | | | | | | | | | | |
| 703 | | | | | | | | | | | | | | | |
| 704 | | | | | | | | | | | | | | | |
| 705 | | | | | | | | | | | | | | | |
| 706 | | | | | | | | | | | | | | | |
| 707 | | | | | | | | | | | | | | | |
| 708 | | | | | | | | | | | | | | | |
| 709 | | | | | | | | | | | | | | | |
| 710 | | | | | | | | | | | | | | | |
| 711 | | | | | | | | | | | | | | | |
| 712 | | | | | | | | | | | | | | | |
| 713 | | | | | | | | | | | | | | | |
| 714 | | | | | | | | | | | | | | | |
| 715 | | | | | | | | | | | | | | | |
| 716 | | | | | | | | | | | | | | | |
| 717 | | | | | | | | | | | | | | | |
| 718 | | | | | | | | | | | | | | | |
| 719 | | | | | | | | | | | | | | | |
| 720 | | | | | | | | | | | | | | | |
| 721 | | | | | | | | | | | | | | | |
| 722 | | | | | | | | | | | | | | | |
| 723 | | | | | | | | | | | | | | | |
| 724 | | | | | | | | | | | | | | | |
| 725 | | | | | | | | | | | | | | | |
| 726 | | | | | | | | | | | | | | | |
| 727 | | | | | | | | | | | | | | | |
| 728 | | | | | | | | | | | | | | | |
| 729 | | | | | | | | | | | | | | | |
| 730 | | | | | | | | | | | | | | | |
| 731 | | | | | | | | | | | | | | | |
| 732 | | | | | | | | | | | | | | | |
| 733 | | | | | | | | | | | | | | | |
| 734 | | | | | | | | | | | | | | | |
| 735 | | | | | | | | | | | | | | | |
| 736 | | | | | | | | | | | | | | | |
| 737 | | | | | | | | | | | | | | | |
| 738 | | | | | | | | | | | | | | | |
| 739 | | | | | | | | | | | | | | | |
| 740 | | | | | | | | | | | | | | | |
| 741 | | | | | | | | | | | | | | | |
| 742 | | | | | | | | | | | | | | | |
| 743 | | | | | | | | | | | | | | | |
| 744 | | | | | | | | | | | | | | | |
| 745 | | | | | | | | | | | | | | | |
| 746 | | | | | | | | | | | | | | | |
| 747 | | | | | | | | | | | | | | | |
| 748 | | | | | | | | | | | | | | | |
| 749 | | | | | | | | | | | | | | | |
| 750 | | | | | | | | | | | | | | | |
| 751 | | | | | | | | | | | | | | | |
| 752 | | | | | | | | | | | | | | | |
| 753 | | | | | | | | | | | | | | | |
| 754 | | | | | | | | | | | | | | | |
| 755 | | | | | | | | | | | | | | | |
| 756 | | | | | | | | | | | | | | | |
| 757 | | | | | | | | | | | | | | | |
| 758 | | | | | | | | | | | | | | | |
| 759 | | | | | | | | | | | | | | | |
| 760 | | | | | | | | | | | | | | | |
| 761 | | | | | | | | | | | | | | | |
| 762 | | | | | | | | | | | | | | | |
| 763 | | | | | | | | | | | | | | | |
| 764 | | | | | | | | | | | | | | | |
| 765 | | | | | | | | | | | | | | | |
| 766 | | | | | | | | | | | | | | | |
| 767 | | | | | | | | | | | | | | | |
| 768 | | | | | | | | | | | | | | | |
| 769 | | | | | | | | | | | | | | | |
| 770 | | | | | | | | | | | | | | | |
| 771 | | | | | | | | | | | | | | | |
| 772 | | | | | | | | | | | | | | | |
| 773 | | | | | | | | | | | | | | | |
| 774 | | | | | | | | | | | | | | | |
| 775 | | | | | | | | | | | | | | | |
| 776 | | | | | | | | | | | | | | | |
| 777 | | | | | | | | | | | | | | | |
| 778 | | | | | | | | | | | | | | | |
| 779 | | | | | | | | | | | | | | | |
| 780 | | | | | | | | | | | | | | | |
| 781 | | | | | | | | | | | | | | | |
| 782 | | | | | | | | | | | | | | | |
| 783 | | | | | | | | | | | | | | | |
| 784 | | | | | | | | | | | | | | | |
| 785 | | | | | | | | | | | | | | | |
| 786 | | | | | | | | | | | | | | | |
| 787 | | | | | | | | | | | | | | | |
| 788 | | | | | | | | | | | | | | | |
| 789 | | | | | | | | | | | | | | | |
| 790 | | | | | | | | | | | | | | | |
| 791 | | | | | | | | | | | | | | | |
| 792 | | | | | | | | | | | | | | | |
| 793 | | | | | | | | | | | | | | | |
| 794 | | | | | | | | | | | | | | | |
| 795 | | | | | | | | | | | | | | | |
| 796 | | | | | | | | | | | | | | | |
| 797 | | | | | | | | | | | | | | | |
| 798 | | | | | | | | | | | | | | | |
| 799 | | | | | | | | | | | | | | | |
| 800 | | | | | | | | | | | | | | | |
| 801 | | | | | | | | | | | | | | | |
| 802 | | | | | | | | | | | | | | | |
| 803 | | | | | | | | | | | | | | | |
| 804 | | | | | | | | | | | | | | | |
| 805 | | | | | | | | | | | | | | | |
| 806 | | | | | | | | | | | | | | | |
| 807 | | | | | | | | | | | | | | | |
| 808 | | | | | | | | | | | | | | | |
| 809 | | | | | | | | | | | | | | | |
| 810 | | | | | | | | | | | | | | | |
| 811 | | | | | | | | | | | | | | | |
| 812 | | | | | | | | | | | | | | | |
| 813 | | | | | | | | | | | | | | | |
| 814 | | | | | | | | | | | | | | | |
| 815 | | | | | | | | | | | | | | | |
| 816 | | | | | | | | | | | | | | | |
| 817 | | | | | | | | | | | | | | | |
| 818 | | | | | | | | | | | | | | | |
| 819 | | | | | | | | | | | | | | | |
| 820 | | | | | | | | | | | | | | | |
| 821 | | | | | | | | | | | | | | | |
| 822 | | | | | | | | | | | | | | | |
| 823 | | | | | | | | | | | | | | | |
| 824 | | | | | | | | | | | | | | | |
| 825 | | | | | | | | | | | | | | | |
| 826 | | | | | | | | | | | | | | | |
| 827 | | | | | | | | | | | | | | | |
| 828 | | | | | | | | | | | | | | | |
| 829 | | | | | | | | | | | | | | | |
| 830 | | | | | | | | | | | | | | | |
| 831 | | | | | | | | | | | | | | | |
| 832 | | | | | | | | | | | | | | | |
| 833 | | | | | | | | | | | | | | | |
| 834 | | | | | | | | | | | | | | | |
| 835 | | | | | | | | | | | | | | | |
| 836 | | | | | | | | | | | | | | | |
| 837 | | | | | | | | | | | | | | | |
| 838 | | | | | | | | | | | | | | | |
| 839 | | | | | | | | | | | | | | | |
| 840 | | | | | | | | | | | | | | | |
| 841 | | | | | | | | | | | | | | | |
| 842 | | | | | | | | | | | | | | | |
| 843 | | | | | | | | | | | | | | | |
| 844 | | | | | | | | | | | | | | | |
| 845 | | | | | | | | | | | | | | | |
| 846 | | | | | | | | | | | | | | | |
| 847 | | | | | | | | | | | | | | | |
| 848 | | | | | | | | | | | | | | | |
| 849 | | | | | | | | | | | | | | | |
| 850 | | | | | | | | | | | | | | | |
| 851 | | | | | | | | | | | | | | | |
| 852 | | | | | | | | | | | | | | | |
| 853 | | | | | | | | | | | | | | | |
| 854 | | | | | | | | | | | | | | | |
| 855 | | | | | | | | | | | | | | | |
| 856 | | | | | | | | | | | | | | | |
| 857 | | | | | | | | | | | | | | | |
| 858 | | | | | | | | | | | | | | | |
| 859 | | | | | | | | | | | | | | | |
| 860 | | | | | | | | | | | | | | | |
| 861 | | | | | | | | | | | | | | | |
| 862 | | | | | | | | | | | | | | | |
| 863 | | | | | | | | | | | | | | | |
| 864 | | | | | | | | | | | | | | | |
| 865 | | | | | | | | | | | | | | | |
| 866 | | | | | | | | | | | | | | | |
| 867 | | | | | | | | | | | | | | | |
| 868 | | | | | | | | | | | | | | | |
| 869 | | | | | | | | | | | | | | | |
| 870 | | | | | | | | | | | | | | | |
| 871 | | | | | | | | | | | | | | | |
| 872 | | | | | | | | | | | | | | | |
| 873 | | | | | | | | | | | | | | | |
| 874 | | | | | | | | | | | | | | | |
| 875 | | | | | | | | | | | | | | | |
| 876 | | | | | | | | | | | | | | | |
| 877 | | | | | | | | | | | | | | | |
| 878 | | | | | | | | | | | | | | | |
| 879 | | | | | | | | | | | | | | | |
| 880 | | | | | | | | | | | | | | | |
| 881 | | | | | | | | | | | | | | | |
| 882 | | | | | | | | | | | | | | | |
| 883 | | | | | | | | | | | | | | | |
| 884 | | | | | | | | | | | | | | | |
| 885 | | | | | | | | | | | | | | | |
| 886 | | | | | | | | | | | | | | | |
| 887 | | | | | | | | | | | | | | | |
| 888 | | | | | | | | | | | | | | | |
| 889 | | | | | | | | | | | | | | | |
| 890 | | | | | | | | | | | | | | | |
| 891 | | | | | | | | | | | | | | | |
| 892 | | | | | | | | | | | | | | | |
| 893 | | | | | | | | | | | | | | | |
| 894 | | | | | | | | | | | | | | | |
| 895 | | | | | | | | | | | | | | | |
| 896 | | | | | | | | | | | | | | | |
| 897 | | | | | | | | | | | | | | | |
| 898 | | | | | | | | | | | | | | | |
| 899 | | | | | | | | | | | | | | | |
| 900 | | | | | | | | | | | | | | | |
| 901 | | | | | | | | | | | | | | | |
| 902 | | | | | | | | | | | | | | | |
| 903 | | | | | | | | | | | | | | | |
| 904 | | | | | | | | | | | | | | | |
| 905 | | | | | | | | | | | | | | | |
| 906 | | | | | | | | | | | | | | | |
| 907 | | | | | | | | | | | | | | | |
| 908 | | | | | | | | | | | | | | | |
| 909 | | | | | | | | | | | | | | | |
| 910 | | | | | | | | | | | | | | | |
| 911 | | | | | | | | | | | | | | | |
| 912 | | | | | | | | | | | | | | | |
| 913 | | | | | | | | | | | | | | | |
| 914 | | | | | | | | | | | | | | | |
| 915 | | | | | | | | | | | | | | | |
| 916 | | | | | | | | | | | | | | | |
| 917 | | | | | | | | | | | | | | | |
| 918 | | | | | | | | | | | | | | | |
| 919 | | | | | | | | | | | | | | | |
| 920 | | | | | | | | | | | | | | | |
| 921 | | | | | | | | | | | | | | | |
| 922 | | | | | | | | | | | | | | | |
| 923 | | | | | | | | | | | | | | | |
| 924 | | | | | | | | | | | | | | | |
| 925 | | | | | | | | | | | | | | | |
| 926 | | | | | | | | | | | | | | | |
| 927 | | | | | | | | | | | | | | | |
| 928 | | | | | | | | | | | | | | | |
| 929 | | | | | | | | | | | | | | | |
| 930 | | | | | | | | | | | | | | | |
| 931 | | | | | | | | | | | | | | | |
| 932 | | | | | | | | | | | | | | | |
| 933 | | | | | | | | | | | | | | | |
| 934 | | | | | | | | | | | | | | | |
| 935 | | | | | | | | | | | | | | | |
| 936 | | | | | | | | | | | | | | | |
| 937 | | | | | | | | | | | | | | | |
| 938 | | | | | | | | | | | | | | | |
| 939 | | | | | | | | | | | | | | | |
| 940 | | | | | | | | | | | | | | | |
| 941 | | | | | | | | | | | | | | | |
| 942 | | | | | | | | | | | | | | | |
| 943 | | | | | | | | | | | | | | | |
| 944 | | | | | | | | | | | | | | | |
| 945 | | | | | | | | | | | | | | | |
| 946 | | | | | | | | | | | | | | | |
| 947 | | | | | | | | | | | | | | | |
| 948 | | | | | | | | | | | | | | | |
| 949 | | | | | | | | | | | | | | | |
| 950 | | | | | | | | | | | | | | | |
| 951 | | | | | | | | | | | | | | | |
| 952 | | | | | | | | | | | | | | | |
| 953 | | | | | | | | | | | | | | | |
| 954 | | | | | | | | | | | | | | | |
| 955 | | | | | | | | | | | | | | | |
| 956 | | | | | | | | | | | | | | | |
| 957 | | | | | | | | | | | | | | | |
| 958 | | | | | | | | | | | | | | | |
| 959 | | | | | | | | | | | | | | | |
| 960 | | | | | | | | | | | | | | | |
| 961 | | | | | | | | | | | | | | | |
| 962 | | | | | | | | | | | | | | | |
| 963 | | | | | | | | | | | | | | | |
| 964 | | | | | | | | | | | | | | | |
| 965 | | | | | | | | | | | | | | | |
| 966 | | | | | | | | | | | | | | | |
| 967 | | | | | | | | | | | | | | | |
| 968 | | | | | | | | | | | | | | | |
| 969 | | | | | | | | | | | | | | | |
| 970 | | | | | | | | | | | | | | | |
| 971 | | | | | | | | | | | | | | | |
| 972 | | | | | | | | | | | | | | | |
| 973 | | | | | | | | | | | | | | | |
| 974 | | | | | | | | | | | | | | | |
| 975 | | | | | | | | | | | | | | | |
| 976 | | | | | | | | | | | | | | | |
| 977 | | | | | | | | | | | | | | | |
| 978 | | | | | | | | | | | | | | | |
| 979 | | | | | | | | | | | | | | | |
| 980 | | | | | | | | | | | | | | | |
| 981 | | | | | | | | | | | | | | | |
| 982 | | | | | | | | | | | | | | | |
| 983 | | | | | | | | | | | | | | | |
| 984 | | | | | | | | | | | | | | | |
| 985 | | | | | | | | | | | | | | | |
| 986 | | | | | | | | | | | | | | | |
| 987 | | | | | | | | | | | | | | | |
| 988 | | | | | | | | | | | | | | | |
| 989 | | | | | | | | | | | | | | | |
| 990 | | | | | | | | | | | | | | | |
| 991 | | | | | | | | | | | | | | | |
| 992 | | | | | | | | | | | | | | | |
| 993 | | | | | | | | | | | | | | | |
| 994 | | | | | | | | | | | | | | | |
| 995 | | | | | | | | | | | | | | | |
| 996 | | | | | | | | | | | | | | | |
| 997 | | | | | | | | | | | | | | | |
| 998 | | | | | | | | | | | | | | | |
| 999 | | | | | | | | | | | | | | | |
| 1000 | | | | | | | | | | | | | | | |
| 1001 | | | | | | | | | | | | | | | |
| 1002 | | | | | | | | | | | | | | | |
| 1003 | | | | | | | | | | | | | | | |
| 1004 | | | | | | | | | | | | | | | |
| 1005 | | | | | | | | | | | | | | | |
| 1006 | | | | | | | | | | | | | | | |
| 1007 | | | | | | | | | | | | | | | |
| 1008 | | | | | | | | | | | | | | | |
| 1009 | | | | | | | | | | | | | | | |
| 1010 | | | | | | | | | | | | | | | |
| 1011 | | | | | | | | | | | | | | | |
| 1012 | | | | | | | | | | | | | | | |
| 1013 | | | | | | | | | | | | | | | |
| 1014 | | | | | | | | | | | | | | | |
| 1015 | | | | | | | | | | | | | | | |
| 1016 | | | | | | | | | | | | | | | |
| 1017 | | | | | | | | | | | | | | | |
| 1018 | | | | | | | | | | | | | | | |
| 1019 | | | | | | | | | | | | | | | |
| 1020 | | | | | | | | | | | | | | | |
| 1021 | | | | | | | | | | | | | | | |
| 1022 | | | | | | | | | | | | | | | |
| 1023 | | | | | | | | | | | | | | | |
| 1024 | | | | | | | | | | | | | | | |
| 1025 | | | | | | | | | | | | | | | |
| 1026 | | | | | | | | | | | | | | | |
| 1027 | | | | | | | | | | | | | | | |
| 1028 | | | | | | | | | | | | | | | |
| 1029 | | | | | | | | | | | | | | | |
| 1030 | | | | | | | | | | | | | | | |
| 1031 | | | | | | | | | | | | | | | |
| 1032 | | | | | | | | | | | | | | | |
| 1033 | | | | | | | | | | | | | | | |
| 1034 | | | | | | | | | | | | | | | |
| 1035 | | | | | | | | | | | | | | | |
| 1036 | | | | | | | | | | | | | | | |
| 1037 | | | | | | | | | | | | | | | |
| 1038 | | | | | | | | | | | | | | | |
| 1039 | | | | | | | | | | | | | | | |
| 1040 | | | | | | | | | | | | | | | |
| 1041 | | | | | | | | | | | | | | | |
| 1042 | | | | | | | | | | | | | | | |
| 1043 | | | | | | | | | | | | | | | |
| 1044 | | | | | | | | | | | | | | | |
| 1045 | | | | | | | | | | | | | | | |
| 1046 | | | | | | | | | | | | | | | |
| 1047 | | | | | | | | | | | | | | | |
| 1048 | | | | | | | | | | | | | | | |
| 1049 | | | | | | | | | | | | | | | |
| 1050 | | | | | | | | | | | | | | | |
| 1051 | | | | | | | | | | | | | | | |
| 1052 | | | | | | | | | | | | | | | |
| 1053 | | | | | | | | | | | | | | | |
| 1054 | | | | | | | | | | | | | | | |
| 1055 | | | | | | | | | | | | | | | |
| 1056 | | | | | | | | | | | | | | | |
| 1057 | | | | | | | | | | | | | | | |
| 1058 | | | | | | | | | | | | | | | |
| 1059 | | | | | | | | | | | | | | | |
| 1060 | | | | | | | | | | | | | | | |
| 1061 | | | | | | | | | | | | | | | |
| 1062 | | | | | | | | | | | | | | | |
| 1063 | | | | | | | | | | | | | | | |
| 1064 | | | | | | | | | | | | | | | |
| 1065 | | | | | | | | | | | | | | | |
| 1066 | | | | | | | | | | | | | | | |
| 1067 | | | | | | | | | | | | | | | |
| 1068 | | | | | | | | | | | | | | | |
| 1069 | | | | | | | | | | | | | | | |
| 1070 | | | | | | | | | | | | | | | |
| 1071 | | | | | | | | | | | | | | | |
| 1072 | | | | | | | | | | | | | | | |
| 1073 | | | | | | | | | | | | | | | |
| 1074 | | | | | | | | | | | | | | | |
| 1075 | | | | | | | | | | | | | | | |
| 1076 | | | | | | | | | | | | | | | |
| 1077 | | | | | | | | | | | | | | | |
| 1078 | | | | | | | | | | | | | | | |
| 1079 | | | | | | | | | | | | | | | |
| 1080 | | | | | | | | | | | | | | | |
| 1081 | | | | | | | | | | | | | | | |
| 1082 | | | | | | | | | | | | | | | |
| 1083 | | | | | | | | | | | | | | | |
| 1084 | | | | | | | | | | | | | | | |
| 1085 | | | | | | | | | | | | | | | |
| 1086 | | | | | | | | | | | | | | | |
| 1087 | | | | | | | | | | | | | | | |
| 1088 | | | | | | | | | | | | | | | |
| 1089 | | | | | | | | | | | | | | | |
| 1090 | | | | | | | | | | | | | | | |
| 1091 | | | | | | | | | | | | | | | |
| 1092 | | | | | | | | | | | | | | | |
| 1093 | | | | | | | | | | | | | | | |
| 1094 | | | | | | | | | | | | | | | |
| 1095 | | | | | | | | | | | | | | | |
| 1096 | | | | | | | | | | | | | | | |
| 1097 | | | | | | | | | | | | | | | |
| 1098 | | | | | | | | | | | | | | | |
| 1099 | | | | | | | | | | | | | | | |
| 1100 | | | | | | | | | | | | | | | |
| 1101 | | | | | | | | | | | | | | | |
| 1102 | | | | | | | | | | | | | | | |
| 1103 | | | | | | | | | | | | | | | |
| 1104 | | | | | | | | | | | | | | | |
| 1105 | | | | | | | | | | | | | | | |
| 1106 | | | | | | | | | | | | | | | |
| 1107 | | | | | | | | | | | | | | | |
| 1108 | | | | | | | | | | | | | | | |
| 1109 | | | | | | | | | | | | | | | |
| 1110 | | | | | | | | | | | | | | | |
| 1111 | | | | | | | | | | | | | | | |
| 1112 | | | | | | | | | | | | | | | |
| 1113 | | | | | | | | | | | | | | | |
| 1114 | | | | | | | | | | | | | | | |
| 1115 | | | | | | | | | | | | | | | |
| 1116 | | | | | | | | | | | | | | | |
| 1117 | | | | | | | | | | | | | | | |
| 1118 | | | | | | | | | | | | | | | |
| 1119 | | | | | | | | | | | | | | | |
| 1120 | | | | | | | | | | | | | | | |
| 1121 | | | | | | | | | | | | | | | |
| 1122 | | | | | | | | | | | | | | | |
| 1123 | | | | | | | | | | | | | | | |
| 1124 | | | | | | | | | | | | | | | |
| 1125 | | | | | | | | | | | | | | | |
| 1126 | | | | | | | | | | | | | | | |
| 1127 | | | | | | | | | | | | | | | |
| 1128 | | | | | | | | | | | | | | | |
| 1129 | | | | | | | | | | | | | | | |
| 1130 | | | | | | | | | | | | | | | |
| 1131 | | | | | | | | | | | | | | | |
| 1132 | | | | | | | | | | | | | | | |
| 1133 | | | | | | | | | | | | | | | |
| 1134 | | | | | | | | | | | | | | | |
| 1135 | | | | | | | | | | | | | | | |
| 1136 | | | | | | | | | | | | | | | |
| 1137 | | | | | | | | | | | | | | | |
| 1138 | | | | | | | | | | | | | | | |
| 1139 | | | | | | | | | | | | | | | |
| 1140 | | | | | | | | | | | | | | | |
| 1141 | | | | | | | | | | | | | | | |
| 1142 | | | | | | | | | | | | | | | |
| 1143 | | | | | | | | | | | | | | | |
| 1144 | | | | | | | | | | | | | | | |
| 1145 | | | | | | | | | | | | | | | |
| 1146 | | | | | | | | | | | | | | | |
| 1147 | | | | | | | | | | | | | | | |
| 1148 | | | | | | | | | | | | | | | |
| 1149 | | | | | | | | | | | | | | | |
| 1150 | | | | | | | | | | | | | | | |
| 1151 | | | | | | | | | | | | | | | |
| 1152 | | | | | | | | | | | | | | | |
| 1153 | | | | | | | | | | | | | | | |
| 1154 | | | | | | | | | | | | | | | |
| 1155 | | | | | | | | | | | | | | | |
| 1156 | | | | | | | | | | | | | | | |
| 1157 | | | | | | | | | | | | | | | |
| 1158 | | | | | | | | | | | | | | | |
| 1159 | | | | | | | | | | | | | | | |
| 1160 | | | | | | | | | | | | | | | |
| 1161 | | | | | | | | | | | | | | | |
| 1162 | | | | | | | | | | | | | | | |
| 1163 | | | | | | | | | | | | | | | |
| 1164 | | | | | | | | | | | | | | | |
| 1165 | | | | | | | | | | | | | | | |
| 1166 | | | | | | | | | | | | | | | |
| 1167 | | | | | | | | | | | | | | | |
| 1168 | | | | | | | | | | | | | | | |
| 1169 | | | | | | | | | | | | | | | |
| 1170 | | | | | | | | | | | | | | | |
| 1171 | | | | | | | | | | | | | | | |
| 1172 | | | | | | | | | | | | | | | |
| 1173 | | | | | | | | | | | | | | | |
| 1174 | | | | | | | | | | | | | | | |
| 1175 | | | | | | | | | | | | | | | |
| 1176 | | | | | | | | | | | | | | | |
| 1177 | | | | | | | | | | | | | | | |
| 1178 | | | | | | | | | | | | | | | |
| 1179 | | | | | | | | | | | | | | | |
| 1180 | | | | | | | | | | | | | | | |
| 1181 | | | | | | | | | | | | | | | |
| 1182 | | | | | | | | | | | | | | | |
| 1183 | | | | | | | | | | | | | | | |
| 1184 | | | | | | | | | | | | | | | |
| 1185 | | | | | | | | | | | | | | | |
| 1186 | | | | | | | | | | | | | | | |
| 1187 | | | | | | | | | | | | | | | |
| 1188 | | | | | | | | | | | | | | | |
| 1189 | | | | | | | | | | | | | | | |
| 1190 | | | | | | | | | | | | | | | |
| 1191 | | | | | | | | | | | | | | | |
| 1192 | | | | | | | | | | | | | | | |
| 1193 | | | | | | | | | | | | | | | |
| 1194 | | | | | | | | | | | | | | | |
| 1195 | | | | | | | | | | | | | | | |
| 1196 | | | | | | | | | | | | | | | |
| 1197 | | | | | | | | | | | | | | | |
| 1198 | | | | | | | | | | | | | | | |
| 1199 | | | | | | | | | | | | | | | |
| 1200 | | | | | | | | | | | | | | | |
| 1201 | | | | | | | | | | | | | | | |
| 1202 | | | | | | | | | | | | | | | |
| 1203 | | | | | | | | | | | | | | | |
| 1204 | | | | | | | | | | | | | | | |
| 1205 | | | | | | | | | | | | | | | |
| 1206 | | | | | | | | | | | | | | | |
| 1207 | | | | | | | | | | | | | | | |
| 1208 | | | | | | | | | | | | | | | |
| 1209 | | | | | | | | | | | | | | | |
| 1210 | | | | | | | | | | | | | | | |
| 1211 | | | | | | | | | | | | | | | |
| 1212 | | | | | | | | | | | | | | | |
| 1213 | | | | | | | | | | | | | | | |
| 1214 | | | | | | | | | | | | | | | |
| 1215 | | | | | | | | | | | | | | | |
| 1216 | | | | | | | | | | | | | | | |
| 1217 | | | | | | | | | | | | | | | |
| 1218 | | | | | | | | | | | | | | | |
| 1219 | | | | | | | | | | | | | | | |
| 1220 | | | | | | | | | | | | | | | |
| 1221 | | | | | | | | | | | | | | | |
| 1222 | | | | | | | | | | | | | | | |
| 1223 | | | | | | | | | | | | | | | |
| 1224 | | | | | | | | | | | | | | | |
| 1225 | | | | | | | | | | | | | | | |
| 1226 | | | | | | | | | | | | | | | |
| 1227 | | | | | | | | | | | | | | | |
| 1228 | | | | | | | | | | | | | | | |
| 1229 | | | | | | | | | | | | | | | |
| 1230 | | | | | | | | | | | | | | | |
| 1231 | | | | | | | | | | | | | | | |
| 1232 | | | | | | | | | | | | | | | |
| 1233 | | | | | | | | | | | | | | | |
| 1234 | | | | | | | | | | | | | | | |
| 1235 | | | | | | | | | | | | | | | |
| 1236 | | | | | | | | | | | | | | | |
| 1237 | | | | | | | | | | | | | | | |
| 1238 | | | | | | | | | | | | | | | |
| 1239 | | | | | | | | | | | | | | | |
| 1240 | | | | | | | | | | | | | | | |
| 1241 | | | | | | | | | | | | | | | |
| 1242 | | | | | | | | | | | | | | | |
| 1243 | | | | | | | | | | | | | | | |
| 1244 | | | | | | | | | | | | | | | |
| 1245 | | | | | | | | | | | | | | | |
| 1246 | | | | | | | | | | | | | | | |
| 1247 | | | | | | | | | | | | | | | |
| 1248 | | | | | | | | | | | | | | | |
| 1249 | | | | | | | | | | | | | | | |
| 1250 | | | | | | | | | | | | | | | |
| 1251 | | | | | | | | | | | | | | | |
| 1252 | | | | | | | | | | | | | | | |
| 1253 | | | | | | | | | | | | | | | |
| 1254 | | | | | | | | | | | | | | | |
| 1255 | | | | | | | | | | | | | | | |
| 1256 | | | | | | | | | | | | | | | |
| 1257 | | | | | | | | | | | | | | | |
| 1258 | | | | | | | | | | | | | | | |
| 1259 | | | | | | | | | | | | | | | |
| 1260 | | | | | | | | | | | | | | | |
| 1261 | | | | | | | | | | | | | | | |
| 1262 | | | | | | | | | | | | | | | |
| 1263 | | | | | | | | | | | | | | | |
| 1264 | | | | | | | | | | | | | | | |
| 1265 | | | | | | | | | | | | | | | |
| 1266 | | | | | | | | | | | | | | | |
| 1267 | | | | | | | | | | | | | | | |
| 1268 | | | | | | | | | | | | | | | |
| 1269 | | | | | | | | | | | | | | | |
| 1270 | | | | | | | | | | | | | | | |
| 1271 | | | | | | | | | | | | | | | |
| 1272 | | | | | | | | | | | | | | | |
| 1273 | | | | | | | | | | | | | | | |
| 1274 | | | | | | | | | | | | | | | |
| 1275 | | | | | | | | | | | | | | | |
| 1276 | | | | | | | | | | | | | | | |
| 1277 | | | | | | | | | | | | | | | |
| 1278 | | | | | | | | | | | | | | | |
| 1279 | | | | | | | | | | | | | | | |
| 1280 | | | | | | | | | | | | | | | |
| 1281 | | | | | | | | | | | | | | | |
| 1282 | | | | | | | | | | | | | | | |
| 1283 | | | | | | | | | | | | | | | |
| 1284 | | | | | | | | | | | | | | | |
| 1285 | | | | | | | | | | | | | | | |
| 1286 | | | | | | | | | | | | | | | |
| 1287 | | | | | | | | | | | | | | | |
| 1288 | | | | | | | | | | | | | | | |
| 1289 | | | | | | | | | | | | | | | |
| 1290 | | | | | | | | | | | | | | | |
| 1291 | | | | | | | | | | | | | | | |
| 1292 | | | | | | | | | | | | | | | |
| 1293 | | | | | | | | | | | | | | | |
| 1294 | | | | | | | | | | | | | | | |
| 1295 | | | | | | | | | | | | | | | |
| 1296 | | | | | | | | | | | | | | | |
| 1297 | | | | | | | | | | | | | | | |
| 1298 | | | | | | | | | | | | | | | |
| 1299 | | | | | | | | | | | | | | | |
| 1300 | | | | | | | | | | | | | | | |
| 1301 | | | | | | | | | | | | | | | |
| 1302 | | | | | | | | | | | | | | | |
| 1303 | | | | | | | | | | | | | | | |
| 1304 | | | | | | | | | | | | | | | |
| 1305 | | | | | | | | | | | | | | | |
| 1306 | | | | | | | | | | | | | | | |
| 1307 | | | | | | | | | | | | | | | |
| 1308 | | | | | | | | | | | | | | | |
| 1309 | | | | | | | | | | | | | | | |
| 1310 | | | | | | | | | | | | | | | |
| 1311 | | | | | | | | | | | | | | | |
| 1312 | | | | | | | | | | | | | | | |
| 1313 | | | | | | | | | | | | | | | |
| 1314 | | | | | | | | | | | | | | | |
| 1315 | | | | | | | | | | | | | | | |
| 1316 | | | | | | | | | | | | | | | |
| 1317 | | | | | | | | | | | | | | | |
| 1318 | | | | | | | | | | | | | | | |
| 1319 | | | | | | | | | | | | | | | |
| 1320 | | | | | | | | | | | | | | | |
| 1321 | | | | | | | | | | | | | | | |
| 1322 | | | | | | | | | | | | | | | |
| 1323 | | | | | | | | | | | | | | | |
| 1324 | | | | | | | | | | | | | | | |
| 1325 | | | | | | | | | | | | | | | |
| 1326 | | | | | | | | | | | | | | | |
| 1327 | | | | | | | | | | | | | | | |
| 1328 | | | | | | | | | | | | | | | |
| 1329 | | | | | | | | | | | | | | | |
| 1330 | | | | | | | | | | | | | | | |
| 1331 | | | | | | | | | | | | | | | |
| 1332 | | | | | | | | | | | | | | | |
| 1333 | | | | | | | | | | | | | | | |
| 1334 | | | | | | | | | | | | | | | |
| 1335 | | | | | | | | | | | | | | | |
| 1336 | | | | | | | | | | | | | | | |
| 1337 | | | | | | | | | | | | | | | |
| 1338 | | | | | | | | | | | | | | | |
| 1339 | | | | | | | | | | | | | | | |
| 1340 | | | | | | | | | | | | | | | |
| 1341 | | | | | | | | | | | | | | | |
| 1342 | | | | | | | | | | | | | | | |
| 1343 | | | | | | | | | | | | | | | |

(Part C)

| | Y | | | | AC | | | | AG | | | | AK | | |
|---|---|---|---|---|---|---|---|---|---|---|---|---|---|---|---|
| | DruD | | | | DruE | | | | DruM | | | | DruF | | |
| | protein | Z | AA | AB | protein | AD | AE | AF | protein | AH | AI | AJ | protein | AL | AM |
| | length | DruE | PRT SEQ | DNA SEQ | length | DruM | PRT SEQ | DNA SEQ | length | DruF | PRT SEQ | DNA SEQ | length | DruG | PRT SEQ |
| 1 | (aa) | protein_accession | ID NO: | ID NO: | (aa) | protein_accession | ID NO: | ID NO: | (aa) | protein_accession | ID NO: | ID NO: | (aa) | protein_accession | ID NO: |
| 2 | | CEP95573.1 | 23937 | 27166 | 1753 | | | | | | | | | | |
| 3 | 363 | KUO96134.1 | 23942 | 27171 | 1851 | | | | | | | | | | |
| 4 | 312 | GAC90454.1 | 23946 | 27175 | 1784 | | | | | | | | | | |
| 5 | 358 | KQT61311.1 | 23949 | 27178 | 1847 | | | | | | | | | | |
| 6 | 358 | KQT62613.1 | 23952 | 27181 | 1847 | | | | | | | | | | |
| 7 | 316 | KMN72584.1 | 23956 | 27185 | 1775 | | | | | | | | | | |
| 8 | 316 | EJQ91446.1 | 23960 | 27189 | 1777 | | | | | | | | | | |
| 9 | 292 | CRK83012.1 | 23964 | 27193 | 1752 | | | | | | | | | | |
| 10 | 333 | KEF29417.1 | 23967 | 27196 | 1743 | | | | | | | | | | |
| 11 | 330 | ETO96255.1 | 23970 | 27199 | 1747 | | | | | | | | | | |
| 12 | 360 | ALK08977.1 | 23973 | 27202 | 1854 | | | | | | | | | | |
| 13 | 360 | CUU41638.1 | 23976 | 27205 | 1854 | | | | | | | | | | |
| 14 | 260 | KGC93324.1 | 23981 | 27210 | 1752 | | | | | | | | | | |
| 15 | 359 | KGV75903.1 | 23984 | 27213 | 1856 | | | | | | | | | | |
| 16 | | EDO89777.1 | 23988 | 27217 | 1787 | | | | | | | | | | |
| 17 | 260 | AJX60137.1 | 23993 | 27222 | 1752 | | | | | | | | | | |
| 18 | | AIP62511.1 | 23996 | 27225 | 1752 | | | | | | | | | | |
| 19 | | KVO65552.1 | 24000 | 27229 | 1752 | | | | | | | | | | |
| 20 | | KVO87082.1 | 24003 | 27232 | 1753 | | | | | | | | | | |
| 21 | 359 | CDI04326.1 | 24006 | 27235 | 1842 | | | | | | | | | | |
| 22 | 351 | KPA09466.1 | 24010 | 27239 | 1795 | | | | | | | | | | |
| 23 | 359 | KHD06508.1 | 24013 | 27242 | 1796 | | | | | | | | | | |
| 24 | 350 | KJH65674.1 | 24018 | 27247 | 1843 | | | | | | | | | | |
| 25 | 340 | CDM70056.1 | 24022 | 27251 | 1761 | | | | | | | | | | |
| 26 | 352 | KEI16343.1 | 24025 | 27254 | 1767 | | | | | | | | | | |
| 27 | 342 | EGB92910.1 | 24029 | 27258 | 1738 | | | | | | | | | | |
| 28 | 339 | ACL18697.1 | 24032 | 27261 | 1740 | | | | | | | | | | |
| 29 | 338 | ACV64030.1 | 24035 | 27264 | 1741 | | | | | | | | | | |
| 30 | 341 | AMC93639.1 | 24038 | 27267 | 1739 | | | | | | | | | | |
| 31 | 347 | KHG78161.1 | 24043 | 27272 | 1836 | | | | | | | | | | |
| 32 | 347 | KHI73839.1 | 24048 | 27277 | 1836 | | | | | | | | | | |
| 33 | 347 | KHI66628.1 | 24051 | 27280 | 1836 | | | | | | | | | | |
| 34 | 347 | KHI48868.1 | 24056 | 27285 | 1836 | | | | | | | | | | |
| 35 | 347 | KHI24186.1 | 24060 | 27289 | 1836 | | | | | | | | | | |
| 36 | 347 | KMV42880.1 | 24065 | 27294 | 1836 | | | | | | | | | | |
| 37 | 347 | KQJ50743.1 | 24070 | 27299 | 1836 | | | | | | | | | | |
| 38 | 347 | ALN47014.1 | 24075 | 27304 | 1836 | | | | | | | | | | |
| 39 | 347 | KSY14480.1 | 24080 | 27309 | 1836 | | | | | | | | | | |
| 40 | 347 | KXR68575.1 | 24085 | 27314 | 1836 | | | | | | | | | | |
| 41 | 347 | KIO85003.1 | 24090 | 27319 | 1836 | | | | | | | | | | |
| 42 | 347 | EQS48047.1 | 24095 | 27324 | 1836 | | | | | | | | | | |
| 43 | 347 | EQO85270.1 | 24100 | 27329 | 1836 | | | | | | | | | | |
| 44 | 347 | EQO74909.1 | 24105 | 27334 | 1836 | | | | | | | | | | |
| 45 | 347 | EYT06142.1 | 24110 | 27339 | 1836 | | | | | | | | | | |
| 46 | 347 | EQV41650.1 | 24115 | 27344 | 1836 | | | | | | | | | | |
| 47 | 347 | ELC24072.1 | 24120 | 27349 | 1836 | | | | | | | | | | |
| 48 | 347 | ELH39088.1 | 24125 | 27354 | 1836 | | | | | | | | | | |
| 49 | 347 | EOV00815.1 | 24130 | 27359 | 1836 | | | | | | | | | | |
| 50 | 347 | EOU57730.1 | 24135 | 27364 | 1836 | | | | | | | | | | |
| 51 | 347 | ELE47062.1 | 24140 | 27369 | 1836 | | | | | | | | | | |
| 52 | 347 | ELE76870.1 | 24145 | 27374 | 1836 | | | | | | | | | | |
| 53 | 347 | EU64787.1 | 24150 | 27379 | 1836 | | | | | | | | | | |
| 54 | 347 | EOV85621.1 | 24155 | 27384 | 1836 | | | | | | | | | | |
| 55 | 347 | ELE86434.1 | 24160 | 27389 | 1836 | | | | | | | | | | |
| 56 | 347 | EWY52042.1 | 24165 | 27394 | 1836 | | | | | | | | | | |
| 57 | 260 | EFK51867.1 | 24170 | 27399 | 1836 | | | | | | | | | | |
| 58 | 260 | EGU96603.1 | 24173 | 27402 | 1836 | | | | | | | | | | |
| 59 | 347 | EMZ39753.1 | 24178 | 27407 | 1836 | | | | | | | | | | |
| 60 | 347 | EGI33544.1 | 24182 | 27411 | 1836 | | | | | | | | | | |
| 61 | 347 | EQX30248.1 | 24187 | 27416 | 1836 | | | | | | | | | | |
| 62 | 347 | EQZ12281.1 | 24192 | 27421 | 1836 | | | | | | | | | | |
| 63 | 347 | ERA40833.1 | 24197 | 27426 | 1836 | | | | | | | | | | |
| 64 | 338 | CDA97614.1 | 24201 | 27430 | 1732 | | | | | | | | | | |
| 65 | 317 | KFX30968.1 | 24205 | 27434 | 1786 | | | | | | | | | | |
| 66 | 317 | KYD25146.1 | 24209 | 27438 | 1786 | | | | | | | | | | |
| 67 | 317 | KLR73617.1 | 24212 | 27441 | 1786 | | | | | | | | | | |
| 68 | 317 | ADU94759.1 | 24216 | 27445 | 1787 | | | | | | | | | | |
| 69 | 317 | ACX76986.1 | 24220 | 27449 | 1787 | | | | | | | | | | |
| 70 | 317 | KFL17493.1 | 24224 | 27453 | 1786 | | | | | | | | | | |
| 71 | 317 | KFX35841.1 | 24228 | 27457 | 1786 | | | | | | | | | | |
| 72 | 365 | EDQ34543.1 | 24231 | 27460 | 1848 | | | | | | | | | | |
| 73 | 360 | KRB96439.1 | 24234 | 27463 | 1851 | | | | | | | | | | |
| 74 | 370 | AGK57209.1 | 24237 | 27466 | 1846 | | | | | | | | | | |
| 75 | 342 | KMV96846.1 | 24242 | 27471 | 1303 | | | | | | | | | | |
| 76 | 354 | ESN04441.1 | 24246 | 27475 | 1846 | | | | | | | | | | |
| 77 | 351 | ABX40975.1 | 24249 | 27478 | 1759 | | | | | | | | | | |
| 78 | 338 | EOS72128.1 | 24253 | 27482 | 1728 | | | | | | | | | | |
| 79 | | EDM25763.1 | 24256 | 27485 | 1813 | | | | | | | | | | |
| 80 | 356 | ESY17005.1 | 24259 | 27488 | 1853 | | | | | | | | | | |
| 81 | 346 | KUK70365.1 | 24263 | 27492 | 1774 | | | | | | | | | | |
| 82 | 343 | EHQ36505.1 | 24267 | 27496 | 1763 | | | | | | | | | | |
| 83 | 347 | KKG83060.1 | 24271 | 27500 | 1775 | | | | | | | | | | |
| 84 | 362 | EQM86810.1 | 24274 | 27503 | 1857 | | | | | | | | | | |
| 85 | 363 | AU18570.1 | 24277 | 27506 | 1858 | | | | | | | | | | |
| 86 | 363 | GAD34406.1 | 24280 | 27509 | 1858 | | | | | | | | | | |
| 87 | 360 | ADJ29891.1 | 24283 | 27512 | 1849 | | | | | | | | | | |
| 88 | 308 | KMP10707.1 | 24286 | 27515 | 1844 | | | | | | | | | | |
| 89 | 354 | EGY00301.1 | 24289 | 27518 | 1849 | | | | | | | | | | |
| 90 | 349 | EPY09172.1 | 24293 | 27522 | 1746 | | | | | | | | | | |
| 91 | 352 | ACX63340.1 | 24297 | 27526 | 1759 | | | | | | | | | | |
| 92 | 347 | KHN90282.1 | 24302 | 27531 | 1836 | | | | | | | | | | |
| 93 | 354 | KGA32596.1 | 24305 | 27534 | 1848 | | | | | | | | | | |
| 94 | 339 | AJQ25680.1 | 24309 | 27538 | 1735 | | | | | | | | | | |
| 95 | 352 | CUH95395.1 | 24313 | 27542 | 1734 | | | | | | | | | | |
| 96 | | KSF97362.1 | 24317 | 27546 | 1840 | | | | | | | | | | |
| 97 | 346 | KSL25394.1 | 24320 | 27549 | 1841 | | | | | | | | | | |
| 98 | | KSO15705.1 | 24324 | 27553 | 1840 | | | | | | | | | | |
| 99 | 353 | BAR67381.2 | 24329 | 27558 | 1844 | | | | | | | | | | |
| 100 | 289 | KAJ21864.1 | 24333 | 27562 | 1802 | | | | | | | | | | |
| 101 | | AGI81183.1 | 24336 | 27565 | 1844 | | | | | | | | | | |
| 102 | 343 | ERY34408.1 | 24341 | 27570 | 1840 | | | | | | | | | | |
| 103 | 353 | EZP23713.1 | 24346 | 27575 | 1844 | | | | | | | | | | |
| 104 | 345 | ERV69459.1 | 24351 | 27580 | 1840 | | | | | | | | | | |
| 105 | | EQM83906.1 | 24355 | 27584 | 1840 | | | | | | | | | | |
| 106 | 344 | AJP52376.1 | 24360 | 27589 | 1848 | | | | | | | | | | |
| 107 | 344 | EST14434.1 | 24365 | 27594 | 1845 | | | | | | | | | | |
| 108 | 343 | ENY74502.1 | 24370 | 27599 | 1840 | | | | | | | | | | |
| 109 | 353 | KWR75282.1 | 24373 | 27602 | 1844 | | | | | | | | | | |
| 110 | 344 | KFE46440.1 | 24378 | 27607 | 1851 | | | | | | | | | | |
| 111 | 371 | AMP72545.1 | 24383 | 27612 | 1853 | | | | | | | | | | |
| 112 | 361 | KQQ11278.1 | 24386 | 27615 | 1860 | | | | | | | | | | |
| 113 | 356 | KQS82594.1 | 24389 | 27618 | 1849 | | | | | | | | | | |
| 114 | 356 | KQS78957.1 | 24392 | 27621 | 1849 | | | | | | | | | | |
| 115 | 345 | AKE89963.1 | 24395 | 27624 | 2149 | | | | | | | | | | |
| 116 | 347 | EME23885.1 | 24398 | 27627 | 1805 | | | | | | | | | | |
| 117 | 364 | CDZ88162.1 | 24402 | 27631 | 2149 | | | | | | | | | | |
| 118 | 355 | ETI62662.1 | 24405 | 27634 | 1847 | | | | | | | | | | |
| 119 | 363 | EFL24975.1 | 24408 | 27637 | 1860 | | | | | | | | | | |
| 120 | 332 | AGA58892.1 | 24411 | 27640 | 1735 | | | | | | | | | | |
| 121 | 343 | EEY51342.1 | 24416 | 27645 | 1831 | | | | | | | | | | |
| 122 | 304 | AIL69853.1 | 24421 | 27650 | 1831 | | | | | | | | | | |
| 123 | 347 | CNL41877.1 | 24426 | 27655 | 1835 | | | | | | | | | | |
| 124 | | CUI54792.1 | 24427 | 27656 | 1891 | CUI54813.1 | 24428 | 27657 | 382 | CUI54751.1 | 24429 | 27658 | 659 | CUI54773.1 | 24430 |
| 125 | | KUL31266.1 | 24431 | 27660 | 1778 | KUL31262.1 | 24432 | 27661 | 427 | KUL31267.1 | 24433 | 27662 | 1018 | | |
| 126 | | AGL13716.1 | 24434 | 27663 | 1783 | AGL13720.1 | 24435 | 27664 | 354 | AGL13717.1 | 24436 | 27665 | 1021 | | |
| 127 | | EGP54151.1 | 24437 | 27666 | 1957 | EGP54154.1 | 24438 | 27667 | 614 | EGP54153.1 | 24439 | 27668 | 822 | EGP54152.1 | 24440 |
| 128 | | ABW19397.1 | 24441 | 27670 | 1903 | ABW19395.1 | 24442 | 27671 | 329 | ABW19399.1 | 24443 | 27672 | 683 | | |
| 129 | | ALO16789.1 | 24444 | 27673 | 2026 | ALO16791.1 | 24445 | 27674 | 390 | ALO16788.1 | 24446 | 27675 | 948 | | |
| 130 | | EIG62888.1 | 24447 | 27676 | 1953 | EIG62885.1 | 24448 | 27677 | 409 | EIG62886.1 | 24449 | 27678 | 852 | EIG62887.1 | 24450 |
| 131 | | ADL01345.1 | 24451 | 27680 | 1954 | ADL01343.1 | 24452 | 27681 | 377 | ADL01347.1 | 24453 | 27682 | 718 | ADL01346.1 | 24454 |
| 132 | | EEE02134.1 | 24455 | 27684 | 1968 | | | | | EEE02132.1 | 24456 | 27685 | 927 | EEE02133.1 | 24457 |
| 133 | | KWF28731.1 | 24458 | 27687 | 1948 | KWF28730.1 | 24459 | 27688 | 391 | KWF28733.1 | 24460 | 27689 | 892 | KWF28732.1 | 24461 |
| 134 | | KQR76945.1 | 24462 | 27691 | 1956 | KQR76951.1 | 24463 | 27692 | 382 | KQR76943.1 | 24464 | 27693 | 844 | KQR76944.1 | 24465 |
| 135 | | KVO29147.1 | 24466 | 27695 | 1947 | KVO29144.1 | 24467 | 27696 | 385 | | | | | KVO29146.1 | 24468 |
| 136 | | KVP15001.1 | 24469 | 27698 | 1947 | KVP14998.1 | 24470 | 27699 | 385 | | | | | KVP15000.1 | 24471 |
| 137 | | KVU67372.1 | 24472 | 27701 | 1948 | KVU67373.1 | 24473 | 27702 | 391 | KVU67370.1 | 24474 | 27703 | 892 | KVU67371.1 | 24475 |
| 138 | | KWH22082.1 | 24476 | 27705 | 1948 | KWH22081.1 | 24477 | 27706 | 391 | KWH22084.1 | 24478 | 27707 | 892 | KWH22083.1 | 24479 |
| 139 | | KWO52778.1 | 24480 | 27709 | 1947 | KWO52775.1 | 24481 | 27710 | 385 | | | | | KWO52777.1 | 24482 |
| 140 | | KQY41878.1 | 24483 | 27712 | 1833 | KQY41875.1 | 24484 | 27713 | 436 | KQY41880.1 | 24485 | 27714 | 692 | | |
| 141 | | ABP36100.1 | 24486 | 27715 | 1887 | ABP36104.1 | 24487 | 27716 | 467 | ABP36098.1 | 24488 | 27717 | 581 | | |
| 142 | | KXH85115.1 | 24489 | 27718 | 2009 | KXH85112.1 | 24490 | 27719 | 377 | KXH85116.1 | 24491 | 27720 | 948 | | |
| 143 | | ALD78636.1 | 24492 | 27721 | 1982 | ALD78633.1 | 24493 | 27722 | 361 | ALD78634.1 | 24494 | 27723 | 921 | ALD78635.1 | 24495 |
| 144 | | EEH92508.2 | 24496 | 27725 | 1947 | EEH92511.1 | 24497 | 27726 | 411 | EEH92510.1 | 24498 | 27727 | 854 | EEH92509.2 | 24499 |
| 145 | | GAL44508.1 | 24500 | 27729 | 1946 | GAL44505.1 | 24501 | 27730 | 395 | GAL44506.1 | 24502 | 27731 | 852 | GAL44507.1 | 24503 |
| 146 | | KUO74508.1 | 24504 | 27733 | 1914 | KUO74511.1 | 24505 | 27734 | 378 | KUO74506.1 | 24506 | 27735 | 675 | | |
| 147 | | AMP07507.1 | 24507 | 27736 | 1971 | AMP07506.1 | 24508 | 27737 | 379 | AMP07509.1 | 24509 | 27738 | 860 | AMP07508.1 | 24510 |
| 148 | | AIF40724.1 | 24511 | 27740 | 1809 | AIF40718.1 | 24512 | 27741 | 331 | AIF40722.1 | 24513 | 27742 | 683 | | |
| 149 | | ACT06326.1 | 24514 | 27743 | 1948 | ACT06329.1 | 24515 | 27744 | 411 | ACT06328.1 | 24516 | 27745 | 853 | ACT06327.1 | 24517 |
| 150 | | KLF19647.1 | 24518 | 27747 | 1982 | KLF19650.1 | 24519 | 27748 | 361 | KLF19649.1 | 24520 | 27749 | 921 | KLF19648.1 | 24521 |
| 151 | | KUQ16675.1 | 24522 | 27751 | 1982 | KUQ16672.1 | 24523 | 27752 | 361 | KUQ16673.1 | 24524 | 27753 | 921 | KUQ16674.1 | 24525 |
| 152 | | KUR17280.1 | 24526 | 27755 | 1982 | KUR17283.1 | 24527 | 27756 | 361 | KUR17282.1 | 24528 | 27757 | 921 | KUR17281.1 | 24529 |
| 153 | | KLG20346.1 | 24530 | 27759 | 1980 | KLG20343.1 | 24531 | 27760 | 361 | | | | | KLG20345.1 | 24532 |
| 154 | | KJL89848.1 | 24533 | 27762 | 1998 | KJL89845.1 | 24534 | 27763 | 361 | KJL89846.1 | 24535 | 27764 | 925 | KJL89847.1 | 24536 |
| 155 | | KJL84453.1 | 24537 | 27766 | 1998 | KJL84456.1 | 24538 | 27767 | 361 | KJL84455.1 | 24539 | 27768 | 925 | KJL84454.1 | 24540 |
| 156 | | KJL75892.1 | 24541 | 27770 | 1998 | KJL75889.1 | 24542 | 27771 | 361 | KJL75890.1 | 24543 | 27772 | 925 | KJL75891.1 | 24544 |
| 157 | | KJX56014.1 | 24545 | 27774 | 1998 | KJX56011.1 | 24546 | 27775 | 361 | KJX56012.1 | 24547 | 27776 | 925 | KJX56013.1 | 24548 |
| 158 | | KJX41325.1 | 24549 | 27778 | 1998 | KJX41322.1 | 24550 | 27779 | 361 | KJX41323.1 | 24551 | 27780 | 925 | KJX41324.1 | 24552 |
| 159 | | KJX62526.1 | 24553 | 27782 | 1998 | KJX62523.1 | 24554 | 27783 | 361 | KJX62524.1 | 24555 | 27784 | 925 | KJX62525.1 | 24556 |
| 160 | | KJX64332.1 | 24557 | 27786 | 1998 | KJX64329.1 | 24558 | 27787 | 361 | KJX64330.1 | 24559 | 27788 | 925 | KJX64331.1 | 24560 |
| 161 | | KJW84168.1 | 24561 | 27790 | 1998 | KJW84165.1 | 24562 | 27791 | 361 | KJW84166.1 | 24563 | 27792 | 925 | KJW84167.1 | 24564 |
| 162 | | KJX04040.1 | 24565 | 27794 | 1998 | KJX04037.1 | 24566 | 27795 | 361 | KJX04038.1 | 24567 | 27796 | 925 | KJX04039.1 | 24568 |
| 163 | | KJW83867.1 | 24569 | 27798 | 1998 | KJW83864.1 | 24570 | 27799 | 361 | KJW83865.1 | 24571 | 27800 | 925 | KJW83866.1 | 24572 |
| 164 | | KJX49731.1 | 24573 | 27802 | 1998 | KJX49728.1 | 24574 | 27803 | 361 | KJX49729.1 | 24575 | 27804 | 925 | KJX49730.1 | 24576 |
| 165 | | KKA32833.1 | 24577 | 27806 | 1998 | KKA32830.1 | 24578 | 27807 | 361 | KKA32831.1 | 24579 | 27808 | 925 | KKA32832.1 | 24580 |
| 166 | | AFM58287.1 | 24581 | 27810 | 1945 | AFM58284.1 | 24582 | 27811 | 411 | AFM58285.1 | 24583 | 27812 | 850 | AFM58286.1 | 24584 |
| 167 | | KTJ18639.1 | 24585 | 27814 | 1998 | KTJ18636.1 | 24586 | 27815 | 361 | KTJ18637.1 | 24587 | 27816 | 925 | KTJ18638.1 | 24588 |
| 168 | | KTH34056.1 | 24589 | 27818 | 1998 | KTH34053.1 | 24590 | 27819 | 361 | KTH34054.1 | 24591 | 27820 | 925 | KTH34055.1 | 24592 |
| 169 | | EWG70005.1 | 24593 | 27822 | 1982 | EWG70008.1 | 24594 | 27823 | 361 | EWG70007.1 | 24595 | 27824 | 921 | EWG70006.1 | 24596 |
| 170 | | BAH40614.1 | 24597 | 27826 | 2009 | BAH40611.1 | 24598 | 27827 | 407 | BAH40612.1 | 24599 | 27828 | 983 | BAH40613.1 | 24600 |
| 171 | | GAB92720.1 | 24601 | 27830 | 1755 | GAB92713.1 | 24602 | 27831 | 422 | GAB92719.1 | 24603 | 27832 | 1025 | | |
| 172 | | KOP23146.1 | 24604 | 27833 | 1766 | KOP23135.1 | 24605 | 27834 | 388 | KOP23134.1 | 24606 | 27835 | 689 | | |
| 173 | | EWE15861.1 | 24607 | 27836 | 1946 | EWE15858.1 | 24608 | 27837 | 381 | EWE15859.1 | 24609 | 27838 | 854 | EWE15860.1 | 24610 |
| 174 | | CDI15591.1 | 24611 | 27840 | 1998 | CDI15594.1 | 24612 | 27841 | 361 | CDI15593.1 | 24613 | 27842 | 925 | CDI15592.1 | 24614 |
| 175 | | EAW41505.1 | 24615 | 27844 | 2067 | EAW41501.1 | 24616 | 27845 | 463 | EAW41506.1 | 24617 | 27846 | 1000 | | |
| 176 | | KQQ96139.1 | 24618 | 27847 | 1983 | KQQ96136.1 | 24619 | 27848 | 363 | KQQ96141.1 | 24620 | 27849 | 940 | KQQ96140.1 | 24621 |
| 177 | | AKB67566.1 | 24622 | 27851 | 2047 | AKB67569.1 | 24623 | 27852 | 489 | AKB67568.1 | 24624 | 27853 | 697 | | |
| 178 | | AIQ93138.1 | 24625 | 27854 | 1960 | AIQ93137.1 | 24626 | 27855 | 340 | AIQ93140.1 | 24627 | 27856 | 925 | AIQ93139.1 | 24628 |
| 179 | | AMB47692.1 | 24629 | 27858 | 1963 | AMB47695.1 | 24630 | 27859 | 315 | AMB47694.1 | 24631 | 27860 | 851 | AMB47693.1 | 24632 |
| 180 | | KIP90348.1 | 24633 | 27862 | 1813 | KIP90388.1 | 24634 | 27863 | 331 | KIP90346.1 | 24635 | 27864 | 683 | | |
| 181 | | EWM65738.1 | 24636 | 27865 | 1812 | EWM65736.1 | 24637 | 27866 | 392 | EWM65739.1 | 24638 | 27867 | 1027 | | |
| 182 | | CCH89535.1 | 24639 | 27868 | 1805 | CCH89539.1 | 24640 | 27869 | 355 | CCH89533.1 | 24641 | 27870 | 668 | | |
| 183 | | EMP50968.1 | 24642 | 27871 | 1945 | EMP50971.1 | 24643 | 27872 | 411 | EMP50970.1 | 24644 | 27873 | 852 | EMP50969.1 | 24645 |
| 184 | | GAS97121.1 | 24646 | 27875 | 1827 | GAS97126.1 | 24647 | 27876 | 329 | GAS97123.1 | 24648 | 27877 | 703 | | |
| 185 | | ALI28820.1 | 24649 | 27878 | 1753 | ALI28823.1 | 24650 | 27879 | 431 | ALI28821.1 | 24651 | 27880 | 1085 | | |
| 186 | | AKS35859.1 | 24652 | 27881 | 1753 | AKS36815.1 | 24653 | 27882 | 431 | AKS35860.1 | 24654 | 27883 | 1031 | | |
| 187 | | GAT08462.1 | 24655 | 27884 | 1822 | GAT08465.1 | 24656 | 27885 | 326 | GAT08464.1 | 24657 | 27886 | 702 | | |
| 188 | | ACV78352.1 | 24658 | 27887 | 1780 | ACV78350.1 | 24659 | 27888 | 467 | ACV78351.1 | 24660 | 27889 | 1002 | | |
| 189 | | KJV46415.1 | 24661 | 27890 | 1946 | KJV46418.1 | 24662 | 27891 | 411 | KJV46417.1 | 24663 | 27892 | 852 | KJV46416.1 | 24664 |
| 190 | | KRW97754.1 | 24665 | 27894 | 1953 | KRW97756.1 | 24666 | 27895 | 603 | | | | | KRW97755.1 | 24667 |
| 191 | | KHT34503.1 | 24668 | 27897 | 1986 | KHT34506.1 | 24669 | 27898 | 361 | KHT34505.1 | 24670 | 27899 | 927 | KHT34504.1 | 24671 |
| 192 | | AFR03569.1 | 24672 | 27901 | 1980 | AFR03572.1 | 24673 | 27902 | 361 | AFR03571.1 | 24674 | 27903 | 921 | AFR03570.1 | 24675 |
| 193 | | AKJ41264.1 | 24676 | 27905 | 1983 | AKJ41261.1 | 24677 | 27906 | 361 | AKJ41262.1 | 24678 | 27907 | 932 | AKJ41263.1 | 24679 |
| 194 | | BAP50995.1 | 24680 | 27909 | 1946 | | | | | BAP50997.1 | 24681 | 27910 | 850 | BAP50996.1 | 24682 |
| 195 | | BAP23387.1 | 24683 | 27912 | 1946 | | | | | BAP23389.1 | 24684 | 27913 | 850 | BAP23388.1 | 24685 |
| 196 | | KSE43346.1 | 24686 | 27915 | 1946 | | | | | KSE43345.1 | 24687 | 27916 | 850 | KSE43344.1 | 24688 |
| 197 | | KSE74811.1 | 24689 | 27918 | 1946 | | | | | KSE74753.1 | 24690 | 27919 | 850 | KSE74754.1 | 24691 |
| 198 | | KSH20463.1 | 24692 | 27921 | 1946 | KSH20466.1 | 24693 | 27922 | 394 | KSH20465.1 | 24694 | 27923 | 850 | KSH20464.1 | 24695 |
| 199 | | KSD81170.1 | 24696 | 27925 | 1946 | KSD81167.1 | 24697 | 27926 | 394 | KSD81166.1 | 24698 | 27927 | 850 | KSD81165.1 | 24699 |
| 200 | | KSD90962.1 | 24700 | 27929 | 1946 | | | | | KSD90904.1 | 24701 | 27930 | 850 | KSD90905.1 | 24702 |
| 201 | | KSH64632.1 | 24703 | 27932 | 1946 | KSH64629.1 | 24704 | 27933 | 394 | KSH64628.1 | 24705 | 27934 | 850 | KSH64627.1 | 24706 |
| 202 | | KSH80104.1 | 24707 | 27936 | 1946 | KSH80101.1 | 24708 | 27937 | 394 | KSH80100.1 | 24709 | 27938 | 850 | KSH80099.1 | 24710 |
| 203 | | KSJ28920.1 | 24711 | 27940 | 1946 | KSJ28917.1 | 24712 | 27941 | 394 | KSJ28916.1 | 24713 | 27942 | 850 | KSJ28915.1 | 24714 |
| 204 | | KSK06443.1 | 24715 | 27944 | 1946 | KSK06442.1 | 24716 | 27945 | 394 | KSK06441.1 | 24717 | 27946 | 850 | KSK06440.1 | 24718 |
| 205 | | KSJ60779.1 | 24719 | 27948 | 1946 | KSJ60711.1 | 24720 | 27949 | 394 | KSJ60712.1 | 24721 | 27950 | 850 | KSJ60713.1 | 24722 |
| 206 | | KSK54149.1 | 24723 | 27952 | 1946 | KSK54146.1 | 24724 | 27953 | 394 | KSK54145.1 | 24725 | 27954 | 850 | KSK54144.1 | 24726 |
| 207 | | KSK39571.1 | 24727 | 27956 | 1946 | KSK39566.1 | 24728 | 27957 | 394 | KSK39565.1 | 24729 | 27958 | 850 | KSK39564.1 | 24730 |
| 208 | | KSL10852.1 | 24731 | 27960 | 1946 | KSL10784.1 | 24732 | 27961 | 394 | KSL10785.1 | 24733 | 27962 | 850 | KSL10786.1 | 24734 |
| 209 | | KSL65229.1 | 24735 | 27964 | 1946 | KSL65226.1 | 24736 | 27965 | 394 | KSL65225.1 | 24737 | 27966 | 850 | KSL65224.1 | 24738 |
| 210 | | KSL37891.1 | 24739 | 27968 | 1946 | KSL37823.1 | 24740 | 27969 | 394 | KSL37824.1 | 24741 | 27970 | 850 | KSL37825.1 | 24742 |
| 211 | | KSM35820.1 | 24743 | 27972 | 1946 | KSM35817.1 | 24744 | 27973 | 394 | KSM35816.1 | 24745 | 27974 | 850 | KSM35815.1 | 24746 |
| 212 | | KSM77524.1 | 24747 | 27976 | 1956 | | | | | KSM77522.1 | 24748 | 27977 | 846 | KSM77523.1 | 24749 |
| 213 | | KSO31601.1 | 24750 | 27979 | 1946 | KSO31533.1 | 24751 | 27980 | 394 | KSO31534.1 | 24752 | 27981 | 850 | KSO31535.1 | 24753 |
| 214 | | KSO28498.1 | 24754 | 27983 | 1946 | KSO28430.1 | 24755 | 27984 | 394 | KSO28431.1 | 24756 | 27985 | 850 | KSO28432.1 | 24757 |
| 215 | | KSL52322.1 | 24758 | 27987 | 1946 | KSL52254.1 | 24759 | 27988 | 394 | KSL52255.1 | 24760 | 27989 | 850 | KSL52256.1 | 24761 |
| 216 | | KSL73599.1 | 24762 | 27991 | 1946 | KSL73596.1 | 24763 | 27992 | 394 | KSL73595.1 | 24764 | 27993 | 850 | KSL73594.1 | 24765 |
| 217 | | KSL62790.1 | 24766 | 27995 | 1956 | | | | | KSL62788.1 | 24767 | 27996 | 846 | KSL62789.1 | 24768 |
| 218 | | KSM90742.1 | 24769 | 27998 | 1946 | KSM90740.1 | 24770 | 27999 | 394 | KSM90739.1 | 24771 | 28000 | 850 | KSM90738.1 | 24772 |
| 219 | | KSN64557.1 | 24773 | 28002 | 1946 | KSN64554.1 | 24774 | 28003 | 394 | KSN64553.1 | 24775 | 28004 | 850 | KSN64552.1 | 24776 |
| 220 | | KSO10606.1 | 24777 | 28006 | 1946 | KSO10538.1 | 24778 | 28007 | 394 | KSO10539.1 | 24779 | 28008 | 850 | KSO10540.1 | 24780 |
| 221 | | KSR13053.1 | 24781 | 28010 | 1946 | KSR12985.1 | 24782 | 28011 | 394 | KSR12986.1 | 24783 | 28012 | 850 | KSR12987.1 | 24784 |
| 222 | | KSR82354.1 | 24785 | 28014 | 1946 | KSR82286.1 | 24786 | 28015 | 394 | KSR82287.1 | 24787 | 28016 | 850 | KSR82288.1 | 24788 |
| 223 | | KSQ84593.1 | 24789 | 28018 | 1946 | KSQ84590.1 | 24790 | 28019 | 394 | KSQ84589.1 | 24791 | 28020 | 850 | KSQ84588.1 | 24792 |
| 224 | | EFQ41826.1 | 24793 | 28022 | 1946 | | | | | EFQ41828.1 | 24794 | 28023 | 850 | EFQ41827.1 | 24795 |
| 225 | | ERX42740.1 | 24796 | 28025 | 1946 | ERX42743.1 | 24797 | 28026 | 379 | ERX42742.1 | 24798 | 28027 | 850 | ERX42741.1 | 24799 |
| 226 | | ERV57398.1 | 24800 | 28029 | 1946 | ERV57401.1 | 24801 | 28030 | 379 | ERV57400.1 | 24802 | 28031 | 850 | ERV57399.1 | 24803 |
| 227 | | ERV46141.1 | 24804 | 28033 | 1946 | ERV46144.1 | 24805 | 28034 | 379 | ERV46143.1 | 24806 | 28035 | 850 | ERV46142.1 | 24807 |
| 228 | | ERV15095.1 | 24808 | 28037 | 1946 | ERV15098.1 | 24809 | 28038 | 379 | ERV15097.1 | 24810 | 28039 | 850 | ERV15096.1 | 24811 |
| 229 | | EZN68207.1 | 24812 | 28041 | 1946 | EZN68210.1 | 24813 | 28042 | 379 | EZN68209.1 | 24814 | 28043 | 850 | EZN68208.1 | 24815 |
| 230 | | EZP10099.1 | 24816 | 28045 | 1956 | EZP10096.1 | 24817 | 28046 | 384 | EZP10097.1 | 24818 | 28047 | 876 | EZP10098.1 | 24819 |
| 231 | | ERW79837.1 | 24820 | 28049 | 1946 | ERW79840.1 | 24821 | 28050 | 379 | ERW79839.1 | 24822 | 28051 | 850 | ERW79838.1 | 24823 |
| 232 | | ERU64537.1 | 24824 | 28053 | 1942 | ERU64534.1 | 24825 | 28054 | 398 | ERU64535.1 | 24826 | 28055 | 839 | ERU64536.1 | 24827 |
| 233 | | ERZ11046.1 | 24828 | 28057 | 1946 | ERZ11049.1 | 24829 | 28058 | 379 | ERZ11048.1 | 24830 | 28059 | 850 | ERZ11047.1 | 24831 |
| 234 | | CDH77378.1 | 24832 | 28061 | 1946 | | | | | CDH77380.1 | 24833 | 28062 | 511 | CDH77379.1 | 24834 |
| 235 | | BAK90082.1 | 24835 | 28064 | 1946 | | | | | BAK90080.1 | 24836 | 28065 | 850 | BAK90081.1 | 24837 |
| 236 | | ERX37642.1 | 24838 | 28067 | 1946 | ERX37645.1 | 24839 | 28068 | 379 | ERX37644.1 | 24840 | 28069 | 850 | ERX37643.1 | 24841 |
| 237 | | CDM51955.1 | 24842 | 28071 | 1946 | CDM51958.1 | 24843 | 28072 | 379 | CDM51957.1 | 24844 | 28073 | 850 | CDM51956.1 | 24845 |
| 238 | | AAY92286.1 | 24846 | 28075 | 1948 | AAY96277.1 | 24847 | 28076 | 394 | AAY92284.1 | 24848 | 28077 | 851 | AAY92285.1 | 24849 |
| 239 | | ELP96357.1 | 24850 | 28079 | 1945 | ELP96354.1 | 24851 | 28080 | 399 | ELP96355.1 | 24852 | 28081 | 838 | ELP96356.1 | 24853 |
| 240 | | ELQ02809.1 | 24854 | 28083 | 1945 | ELQ02806.1 | 24855 | 28084 | 399 | ELQ02807.1 | 24856 | 28085 | 838 | ELQ02808.1 | 24857 |
| 241 | | KGK94204.1 | 24858 | 28087 | 1957 | KGK94314.1 | 24859 | 28088 | 406 | KGK94207.1 | 24860 | 28089 | 847 | KGK94205.1 | 24861 |
| 242 | | KUR43160.1 | 24862 | 28091 | 1957 | KUR43163.1 | 24863 | 28092 | 384 | KUR43162.1 | 24864 | 28093 | 847 | KUR43161.1 | 24865 |
| 243 | | KUR49796.1 | 24866 | 28095 | 1957 | KUR49793.1 | 24867 | 28096 | 384 | KUR49794.1 | 24868 | 28097 | 847 | KUR49795.1 | 24869 |
| 244 | | EEB59176.1 | 24870 | 28099 | 1957 | EEB59179.1 | 24871 | 28100 | 406 | EEB59178.1 | 24872 | 28101 | 847 | EEB59177.1 | 24873 |
| 245 | | ERL54332.1 | 24874 | 28103 | 1930 | | | | | ERL54330.1 | 24875 | 28104 | 845 | ERL54331.1 | 24876 |
| 246 | | KPH05039.1 | 24877 | 28106 | 1961 | KPH05036.1 | 24878 | 28107 | 353 | KPH05037.1 | 24879 | 28108 | 854 | KPH05038.1 | 24880 |
| 247 | | KQS76378.1 | 24881 | 28110 | 1626 | | | | | KQS76380.1 | 24882 | 28111 | 853 | KQS76379.1 | 24883 |
| 248 | | KUJ85953.1 | 24884 | 28113 | 1804 | KUJ86243.1 | 24885 | 28114 | 472 | KUJ85952.1 | 24886 | 28115 | 1026 | | |
| 249 | | KTM77905.1 | 24887 | 28116 | 1980 | KTM77908.1 | 24888 | 28117 | 361 | KTM77907.1 | 24889 | 28118 | 921 | KTM77906.1 | 24890 |
| 250 | | GAR19711.1 | 24891 | 28120 | 1945 | GAR19708.1 | 24892 | 28121 | 395 | GAR19709.1 | 24893 | 28122 | 852 | GAR19710.1 | 24894 |
| 251 | | GAR29094.1 | 24895 | 28124 | 1945 | GAR29097.1 | 24896 | 28125 | 395 | GAR29096.1 | 24897 | 28126 | 852 | GAR29095.1 | 24898 |
| 252 | | GAR48964.1 | 24899 | 28128 | 1945 | GAR48967.1 | 24900 | 28129 | 395 | GAR48966.1 | 24901 | 28130 | 852 | GAR48965.1 | 24902 |
| 253 | | GAR51851.1 | 24903 | 28132 | 1980 | GAR51848.1 | 24904 | 28133 | 361 | GAR51849.1 | 24905 | 28134 | 921 | GAR51850.1 | 24906 |
| 254 | | GAR68135.1 | 24907 | 28136 | 1945 | GAR68138.1 | 24908 | 28137 | 395 | GAR68137.1 | 24909 | 28138 | 852 | GAR68136.1 | 24910 |
| 255 | | GAR79837.1 | 24911 | 28140 | 1945 | GAR79834.1 | 24912 | 28141 | 395 | GAR79835.1 | 24913 | 28142 | 852 | GAR79836.1 | 24914 |
| 256 | | GAS04635.1 | 24915 | 28144 | 1945 | GAS04638.1 | 24916 | 28145 | 395 | GAS04637.1 | 24917 | 28146 | 852 | GAS04636.1 | 24918 |
| 257 | | GAS14633.1 | 24919 | 28148 | 1945 | GAS14636.1 | 24920 | 28149 | 395 | GAS14635.1 | 24921 | 28150 | 852 | GAS14634.1 | 24922 |
| 258 | | GAS22419.1 | 24923 | 28152 | 1945 | GAS22416.1 | 24924 | 28153 | 395 | GAS22417.1 | 24925 | 28154 | 852 | GAS22418.1 | 24926 |
| 259 | | GAS34224.1 | 24927 | 28156 | 1945 | GAS34221.1 | 24928 | 28157 | 395 | GAS34222.1 | 24929 | 28158 | 852 | GAS34223.1 | 24930 |
| 260 | | GAS44702.1 | 24931 | 28160 | 1945 | GAS44699.1 | 24932 | 28161 | 395 | GAS44700.1 | 24933 | 28162 | 852 | GAS44701.1 | 24934 |
| 261 | | KSB75754.1 | 24935 | 28164 | 1945 | KSB75751.1 | 24936 | 28165 | 411 | KSB75752.1 | 24937 | 28166 | 849 | KSB75753.1 | 24938 |
| 262 | | AIP97316.1 | 24939 | 28168 | 1945 | AIP94652.1 | 24940 | 28169 | 411 | AIP97314.1 | 24941 | 28170 | 849 | AIP97315.1 | 24942 |
| 263 | | KFS83334.1 | 24943 | 28172 | 1980 | KFS83337.1 | 24944 | 28173 | 361 | KFS83336.1 | 24945 | 28174 | 921 | KFS83335.1 | 24946 |
| 264 | | KFS82350.1 | 24947 | 28176 | 1980 | | | | | KFS82348.1 | 24948 | 28177 | 617 | KFS82349.1 | 24949 |
| 265 | | KFS54100.1 | 24950 | 28179 | 1980 | KFS54103.1 | 24951 | 28180 | 361 | KFS54102.1 | 24952 | 28181 | 921 | KFS54101.1 | 24953 |
| 266 | | ETA85463.1 | 24954 | 28183 | 1946 | ETA85466.1 | 24955 | 28184 | 411 | ETA85465.1 | 24956 | 28185 | 852 | ETA85464.1 | 24957 |
| 267 | | AGQ75543.1 | 24958 | 28187 | 1946 | AGQ73595.1 | 24959 | 28188 | 395 | AGQ75541.1 | 24960 | 28189 | 852 | AGQ75542.1 | 24961 |
| 268 | | ESV53477.1 | 24962 | 28191 | 1946 | ESV53381.1 | 24963 | 28192 | 395 | ESV53475.1 | 24964 | 28193 | 852 | ESV53476.1 | 24965 |
| 269 | | ESJ68059.1 | 24966 | 28195 | 1980 | ESJ68062.1 | 24967 | 28196 | 361 | ESJ68061.1 | 24968 | 28197 | 921 | ESJ68060.1 | 24969 |
| 270 | | ESE97307.1 | 24970 | 28199 | 1980 | ESE97310.1 | 24971 | 28200 | 361 | ESE97309.1 | 24972 | 28201 | 921 | ESE97308.1 | 24973 |
| 271 | | EDZ02526.1 | 24974 | 28203 | 1980 | EDZ04146.1 | 24975 | 28204 | 361 | | | | | EDZ04762.1 | 24976 |
| 272 | | ETO88599.1 | 24977 | 28206 | 1980 | ETO88602.1 | 24978 | 28207 | 361 | ETO88601.1 | 24979 | 28208 | 921 | ETO88600.1 | 24980 |
| 273 | | CUS01271.1 | 24981 | 28210 | 1982 | CUS01249.1 | 24982 | 28211 | 361 | CUS01256.1 | 24983 | 28212 | 921 | CUS01267.1 | 24984 |
| 274 | | CUR99590.1 | 24985 | 28214 | 1982 | CUR99579.1 | 24986 | 28215 | 361 | CUR99583.1 | 24987 | 28216 | 921 | CUR99587.1 | 24988 |
| 275 | | CUR99591.1 | 24989 | 28218 | 1982 | CUR99580.1 | 24990 | 28219 | 361 | CUR99582.1 | 24991 | 28220 | 921 | CUR99586.1 | 24992 |
| 276 | | CUR99699.1 | 24993 | 28222 | 1982 | CUR99684.1 | 24994 | 28223 | 361 | CUR99689.1 | 24995 | 28224 | 921 | CUR99695.1 | 24996 |
| 277 | | CBY98492.1 | 24997 | 28226 | 1982 | CBY98489.1 | 24998 | 28227 | 361 | CBY98490.1 | 24999 | 28228 | 921 | CBY98491.1 | 25000 |
| 278 | | EDZ28883.1 | 25001 | 28230 | 1982 | EDZ28906.1 | 25002 | 28231 | 361 | EDZ28845.1 | 25003 | 28232 | 921 | EDZ28831.1 | 25004 |
| 279 | | KHQ80350.1 | 25005 | 28234 | 1946 | KHQ80347.1 | 25006 | 28235 | 411 | KHQ80348.1 | 25007 | 28236 | 852 | KHQ80349.1 | 25008 |
| 280 | | ESE86334.1 | 25009 | 28238 | 1946 | ESE86332.1 | 25010 | 28239 | 411 | | | | | ESE86333.1 | 25011 |
| 281 | | ETX40265.1 | 25012 | 28241 | 1947 | ETX40262.1 | 25013 | 28242 | 411 | ETX40263.1 | 25014 | 28243 | 852 | ETX40264.1 | 25015 |
| 282 | | EZQ62320.1 | 25016 | 28245 | 1947 | EZQ62323.1 | 25017 | 28246 | 411 | EZQ62322.1 | 25018 | 28247 | 852 | EZQ62321.1 | 25019 |
| 283 | | CAC47725.1 | 25020 | 28249 | 1832 | CAC47727.1 | 25021 | 28250 | 440 | CAC47726.1 | 25022 | 28251 | 997 | | |
| 284 | | AGG75744.1 | 25023 | 28252 | 1832 | AGG75746.1 | 25024 | 28253 | 440 | AGG75745.1 | 25025 | 28254 | 997 | | |
| 285 | | KSV87560.1 | 25026 | 28255 | 1961 | KSV87563.1 | 25027 | 28256 | 383 | KSV87562.1 | 25028 | 28257 | 854 | KSV87561.1 | 25029 |
| 286 | | EJJ06042.1 | 25030 | 28259 | 1849 | EJJ06043.1 | 25031 | 28260 | 405 | EJJ06040.1 | 25032 | 28261 | 705 | | |
| 287 | | KND14004.1 | 25033 | 28262 | 1857 | KND14010.1 | 25034 | 28263 | 405 | KND14006.1 | 25035 | 28264 | 712 | | |
| 288 | | EGU44860.1 | 25036 | 28265 | 2093 | EGU44862.1 | 25037 | 28266 | 528 | EGU44859.1 | 25038 | 28267 | 1014 | | |
| 289 | | CNL16915.1 | 25039 | 28268 | 1981 | CNL16836.1 | 25040 | 28269 | 362 | CNL16872.1 | 25041 | 28270 | 928 | CNL16894.1 | 25042 |
| 290 | | CNJ89349.1 | 25043 | 28272 | 1981 | CNJ89455.1 | 25044 | 28273 | 362 | CNJ89414.1 | 25045 | 28274 | 928 | CNJ89387.1 | 25046 |
| 291 | | CNK54575.1 | 25047 | 28276 | 1982 | CNK54663.1 | 25048 | 28277 | 376 | CNK54635.1 | 25049 | 28278 | 927 | CNK54608.1 | 25050 |
| 292 | | CNK94601.1 | 25051 | 28280 | 1981 | CNK94669.1 | 25052 | 28281 | 362 | CNK94646.1 | 25053 | 28282 | 928 | CNK94618.1 | 25054 |
| 293 | | CNI43727.1 | 25055 | 28284 | 1982 | CNI43814.1 | 25056 | 28285 | 376 | CNI43794.1 | 25057 | 28286 | 927 | CNI43767.1 | 25058 |
| 294 | | AJJ11891.1 | 25059 | 28288 | 1986 | AJJ11355.1 | 25060 | 28289 | 362 | AJJ10816.1 | 25061 | 28290 | 928 | AJJ11516.1 | 25062 |
| 295 | | EEQ02997.1 | 25063 | 28292 | 1986 | EEQ03000.1 | 25064 | 28293 | 362 | EEQ02999.1 | 25065 | 28294 | 928 | EEQ02998.1 | 25066 |
| 296 | | KXV70696.1 | 25067 | 28296 | 2166 | | | | | | | | | | |
| 297 | | AKQ30571.1 | 25069 | 28298 | 2105 | | | | | | | | | | |
| 298 | | KOP86148.1 | 25071 | 28300 | 2105 | | | | | | | | | | |
| 299 | | KRS04352.1 | 25073 | 28302 | 2105 | | | | | | | | | | |
| 300 | | KRS02342.1 | 25075 | 28304 | 2105 | | | | | | | | | | |
| 301 | | KRS03107.1 | 25077 | 28306 | 2105 | | | | | | | | | | |
| 302 | | KRR94714.1 | 25079 | 28308 | 2105 | | | | | | | | | | |
| 303 | | KRS09258.1 | 25081 | 28310 | 2105 | | | | | | | | | | |
| 304 | | EXE63524.1 | 25083 | 28312 | 2107 | | | | | | | | | | |
| 305 | | EXC47766.1 | 25085 | 28314 | 2099 | | | | | | | | | | |
| 306 | | ELW92979.1 | 25087 | 28316 | 2107 | | | | | | | | | | |
| 307 | | ENW35933.1 | 25089 | 28318 | 2105 | | | | | | | | | | |
| 308 | | ENW44602.1 | 25091 | 28320 | 2105 | | | | | | | | | | |
| 309 | | ENW49012.1 | 25093 | 28322 | 2098 | | | | | | | | | | |
| 310 | | EKP68370.1 | 25095 | 28324 | 2098 | | | | | | | | | | |
| 311 | | EKL42013.1 | 25097 | 28326 | 2105 | | | | | | | | | | |
| 312 | | ENV15228.1 | 25099 | 28328 | 2099 | | | | | | | | | | |
| 313 | | ENX21439.1 | 25101 | 28330 | 2105 | | | | | | | | | | |
| 314 | | ENV06099.1 | 25103 | 28332 | 2099 | | | | | | | | | | |
| 315 | | EDS03747.1 | 25105 | 28334 | 2047 | | | | | | | | | | |
| 316 | | AMN11450.1 | 25107 | 28336 | 2100 | | | | | | | | | | |
| 317 | | AFT94989.1 | 25109 | 28338 | 2100 | | | | | | | | | | |
| 318 | | EGF91079.1 | 25111 | 28340 | 2037 | | | | | | | | | | |
| 319 | | ESQ74519.1 | 25113 | 28342 | 2035 | | | | | | | | | | |
| 320 | | CAL94925.1 | 25115 | 28344 | 2109 | | | | | | | | | | |
| 321 | | AJE23874.1 | 25117 | 28346 | 1705 | | | | | | | | | | |
| 322 | | CUA18545.1 | 25119 | 28348 | 2048 | | | | | | | | | | |
| 323 | | CBW22536.1 | 25121 | 28350 | 2048 | | | | | | | | | | |
| 324 | | EYA28061.1 | 25123 | 28352 | 2046 | | | | | | | | | | |
| 325 | | EYA33839.1 | 25125 | 28354 | 2048 | | | | | | | | | | |
| 326 | | EXY44320.1 | 25127 | 28356 | 2048 | | | | | | | | | | |
| 327 | | EYB10318.1 | 25129 | 28358 | 2048 | | | | | | | | | | |
| 328 | | EXZ67979.1 | 25131 | 28360 | 2048 | | | | | | | | | | |
| 329 | | EXY49048.1 | 25133 | 28362 | 2048 | | | | | | | | | | |
| 330 | | EXY53777.1 | 25135 | 28364 | 2048 | | | | | | | | | | |
| 331 | | EDS16320.1 | 25137 | 28366 | 2047 | | | | | | | | | | |
| 332 | | KXT38969.1 | 25139 | 28368 | 2047 | | | | | | | | | | |
| 333 | | EGW44032.1 | 25141 | 28370 | 2053 | | | | | | | | | | |
| 334 | | EEA03717.1 | 25143 | 28372 | 2083 | | | | | | | | | | |
| 335 | | KVM07646.1 | 25145 | 28374 | 2080 | | | | | | | | | | |
| 336 | | KVM11423.1 | 25147 | 28376 | 2080 | | | | | | | | | | |
| 337 | | KVM41744.1 | 25149 | 28378 | 2080 | | | | | | | | | | |
| 338 | | KVU39940.1 | 25151 | 28380 | 2084 | | | | | | | | | | |
| 339 | | KVX50941.1 | 25153 | 28382 | 2080 | | | | | | | | | | |
| 340 | | CDH43330.1 | 25155 | 28384 | 2062 | | | | | | | | | | |
| 341 | | KPA18787.1 | 25157 | 28386 | 2057 | | | | | | | | | | |
| 342 | | AIY12566.1 | 25159 | 28388 | 1978 | | | | | | | | | | |
| 343 | | EYF08672.1 | 25161 | 28390 | 1820 | | | | | | | | | | |
| 344 | | KQM35753.1 | 25163 | 28392 | 1956 | | | | | | | | | | |
| 345 | | ALD78156.1 | 25165 | 28394 | 2108 | | | | | | | | | | |
| 346 | | KPR54498.1 | 25167 | 28396 | 2106 | | | | | | | | | | |
| 347 | | KLV49873.1 | 25169 | 28398 | 2162 | | | | | | | | | | |
| 348 | | KLV71165.1 | 25171 | 28400 | 2106 | | | | | | | | | | |
| 349 | | BAO81224.1 | 25173 | 28402 | 2099 | | | | | | | | | | |
| 350 | | ALB51925.1 | 25175 | 28404 | 2106 | | | | | | | | | | |
| 351 | | AGE87745.1 | 25177 | 28406 | 1380 | | | | | | | | | | |
| 352 | | KJR98717.1 | 25179 | 28408 | 2076 | | | | | | | | | | |
| 353 | | KLE94551.1 | 25181 | 28410 | 2106 | | | | | | | | | | |
| 354 | | KLF52638.1 | 25183 | 28412 | 2106 | | | | | | | | | | |
| 355 | | KLW28645.1 | 25185 | 28414 | 2106 | | | | | | | | | | |
| 356 | | KLW04619.1 | 25187 | 28416 | 2106 | | | | | | | | | | |
| 357 | | KLQ05990.1 | 25189 | 28418 | 2108 | | | | | | | | | | |
| 358 | | KJI56236.1 | 25191 | 28420 | 2108 | | | | | | | | | | |
| 359 | | KLP80553.1 | 25193 | 28422 | 2106 | | | | | | | | | | |
| 360 | | KLW09755.1 | 25195 | 28424 | 2106 | | | | | | | | | | |
| 361 | | KLW05113.1 | 25197 | 28426 | 2106 | | | | | | | | | | |
| 362 | | KUQ64543.1 | 25199 | 28428 | 2080 | | | | | | | | | | |
| 363 | | KUQ79081.1 | 25201 | 28430 | 2080 | | | | | | | | | | |
| 364 | | KPS33776.1 | 25203 | 28432 | 2106 | | | | | | | | | | |
| 365 | | ESL79957.1 | 25205 | 28434 | 2106 | | | | | | | | | | |
| 366 | | KLW78405.1 | 25207 | 28436 | 2106 | | | | | | | | | | |
| 367 | | KFC20758.1 | 25209 | 28438 | 1950 | | | | | | | | | | |
| 368 | | AKQ40859.1 | 25211 | 28440 | 1989 | | | | | | | | | | |
| 369 | | EDL49427.1 | 25213 | 28442 | 1988 | | | | | | | | | | |
| 370 | | KDM84272.1 | 25215 | 28444 | 2104 | | | | | | | | | | |
| 371 | | CDU40545.1 | 25217 | 28446 | 2114 | | | | | | | | | | |
| 372 | | KHJ28072.1 | 25219 | 28448 | 2114 | | | | | | | | | | |
| 373 | | KHI41602.1 | 25221 | 28450 | 2104 | | | | | | | | | | |
| 374 | | KHH60282.1 | 25223 | 28452 | 2114 | | | | | | | | | | |
| 375 | | KHH50418.1 | 25225 | 28454 | 2114 | | | | | | | | | | |
| 376 | | KHI22226.1 | 25227 | 28456 | 2104 | | | | | | | | | | |
| 377 | | KHH70907.1 | 25229 | 28458 | 2114 | | | | | | | | | | |
| 378 | | KIG29571.1 | 25231 | 28460 | 2104 | | | | | | | | | | |
| 379 | | AJE58952.1 | 25233 | 28462 | 2114 | | | | | | | | | | |
| 380 | | KIY28272.1 | 25235 | 28464 | 2104 | | | | | | | | | | |
| 381 | | KIZ09778.1 | 25237 | 28466 | 2104 | | | | | | | | | | |
| 382 | | KJJ46593.1 | 25239 | 28468 | 2104 | | | | | | | | | | |
| 383 | | KJW55354.1 | 25241 | 28470 | 2104 | | | | | | | | | | |
| 384 | | KJW51696.1 | 25243 | 28472 | 2104 | | | | | | | | | | |
| 385 | | KLG52811.1 | 25245 | 28474 | 2104 | | | | | | | | | | |
| 386 | | KLX28162.1 | 25247 | 28476 | 2114 | | | | | | | | | | |
| 387 | | KNF16836.1 | 25249 | 28478 | 2104 | | | | | | | | | | |
| 388 | | KNF20995.1 | 25251 | 28480 | 2104 | | | | | | | | | | |
| 389 | | KNF39640.1 | 25253 | 28482 | 2104 | | | | | | | | | | |
| 390 | | KNG40958.1 | 25255 | 28484 | 2104 | | | | | | | | | | |
| 391 | | KNZ99111.1 | 25257 | 28486 | 2114 | | | | | | | | | | |
| 392 | | KOZ12765.1 | 25259 | 28488 | 2104 | | | | | | | | | | |
| 393 | | KOZ62647.1 | 25261 | 28490 | 2104 | | | | | | | | | | |
| 394 | | KOZ92031.1 | 25263 | 28492 | 2104 | | | | | | | | | | |
| 395 | | KOZ11137.1 | 25265 | 28494 | 2104 | | | | | | | | | | |
| 396 | | KOZ14566.1 | 25267 | 28496 | 2104 | | | | | | | | | | |
| 397 | | KOZ32809.1 | 25269 | 28498 | 2104 | | | | | | | | | | |
| 398 | | KOZ23191.1 | 25271 | 28500 | 2104 | | | | | | | | | | |
| 399 | | KOZ33147.1 | 25273 | 28502 | 2104 | | | | | | | | | | |
| 400 | | KOZ37736.1 | 25275 | 28504 | 2104 | | | | | | | | | | |
| 401 | | KOZ50072.1 | 25277 | 28506 | 2104 | | | | | | | | | | |
| 402 | | KOZ47195.1 | 25279 | 28508 | 2104 | | | | | | | | | | |
| 403 | | KOZ49645.1 | 25281 | 28510 | 2104 | | | | | | | | | | |
| 404 | | KOZ66201.1 | 25283 | 28512 | 2104 | | | | | | | | | | |
| 405 | | KOZ69676.1 | 25285 | 28514 | 2104 | | | | | | | | | | |
| 406 | | KOZ74923.1 | 25287 | 28516 | 2104 | | | | | | | | | | |
| 407 | | KOZ84735.1 | 25289 | 28518 | 2104 | | | | | | | | | | |
| 408 | | KOZ80210.1 | 25291 | 28520 | 2104 | | | | | | | | | | |
| 409 | | KOZ97677.1 | 25293 | 28522 | 2104 | | | | | | | | | | |
| 410 | | CTS44204.1 | 25295 | 28524 | 2116 | | | | | | | | | | |
| 411 | | KPH47383.1 | 25297 | 28526 | 2104 | | | | | | | | | | |
| 412 | | KPH45899.1 | 25299 | 28528 | 2104 | | | | | | | | | | |
| 413 | | KPH35130.1 | 25301 | 28530 | 2104 | | | | | | | | | | |
| 414 | | KPH38257.1 | 25303 | 28532 | 2104 | | | | | | | | | | |
| 415 | | KPO74170.1 | 25305 | 28534 | 2104 | | | | | | | | | | |
| 416 | | KPP03700.1 | 25307 | 28536 | 2104 | | | | | | | | | | |
| 417 | | KPP08799.1 | 25309 | 28538 | 2104 | | | | | | | | | | |
| 418 | | KPP18124.1 | 25311 | 28540 | 2104 | | | | | | | | | | |
| 419 | | KPP23767.1 | 25313 | 28542 | 2104 | | | | | | | | | | |
| 420 | | KPP34281.1 | 25315 | 28544 | 2104 | | | | | | | | | | |
| 421 | | KPP38851.1 | 25317 | 28546 | 2104 | | | | | | | | | | |
| 422 | | KPP49039.1 | 25319 | 28548 | 2104 | | | | | | | | | | |
| 423 | | KPP10067.1 | 25321 | 28550 | 2104 | | | | | | | | | | |
| 424 | | KPP41789.1 | 25323 | 28552 | 2104 | | | | | | | | | | |
| 425 | | KPP53630.1 | 25325 | 28554 | 2104 | | | | | | | | | | |
| 426 | | KUH02052.1 | 25327 | 28556 | 2104 | | | | | | | | | | |
| 427 | | KUH15976.1 | 25329 | 28558 | 2114 | | | | | | | | | | |
| 428 | | KUR31930.1 | 25331 | 28560 | 2114 | | | | | | | | | | |
| 429 | | ALZ66920.1 | 25333 | 28562 | 2114 | | | | | | | | | | |
| 430 | | KUT20744.1 | 25335 | 28564 | 2114 | | | | | | | | | | |
| 431 | | KUT66046.1 | 25337 | 28566 | 2114 | | | | | | | | | | |
| 432 | | KUS25060.1 | 25339 | 28568 | 2114 | | | | | | | | | | |
| 433 | | KUS40192.1 | 25341 | 28570 | 2104 | | | | | | | | | | |
| 434 | | KUT25847.1 | 25343 | 28572 | 2104 | | | | | | | | | | |
| 435 | | KUT18408.1 | 25345 | 28574 | 2104 | | | | | | | | | | |
| 436 | | KUU15347.1 | 25347 | 28576 | 2114 | | | | | | | | | | |
| 437 | | KUT50287.1 | 25349 | 28578 | 2104 | | | | | | | | | | |
| 438 | | KUX11783.1 | 25351 | 28580 | 2114 | | | | | | | | | | |
| 439 | | KUU59436.1 | 25353 | 28582 | 2114 | | | | | | | | | | |
| 440 | | KUU32865.1 | 25355 | 28584 | 2104 | | | | | | | | | | |
| 441 | | KUU24011.1 | 25357 | 28586 | 2104 | | | | | | | | | | |
| 442 | | KUV87992.1 | 25359 | 28588 | 2104 | | | | | | | | | | |
| 443 | | KUW19154.1 | 25361 | 28590 | 2104 | | | | | | | | | | |
| 444 | | KUW69105.1 | 25363 | 28592 | 2104 | | | | | | | | | | |
| 445 | | KUW47130.1 | 25365 | 28594 | 2104 | | | | | | | | | | |
| 446 | | KUT58081.1 | 25367 | 28596 | 2114 | | | | | | | | | | |
| 447 | | KUX52751.1 | 25369 | 28598 | 2114 | | | | | | | | | | |
| 448 | | KUX36550.1 | 25371 | 28600 | 2114 | | | | | | | | | | |
| 449 | | AMG80375.1 | 25373 | 28602 | 2104 | | | | | | | | | | |
| 450 | | KXG88717.1 | 25375 | 28604 | 2090 | | | | | | | | | | |
| 451 | | KXK90271.1 | 25377 | 28606 | 2104 | | | | | | | | | | |
| 452 | | KXP75339.1 | 25379 | 28608 | 2104 | | | | | | | | | | |
| 453 | | KXP56977.1 | 25381 | 28610 | 2114 | | | | | | | | | | |
| 454 | | KXP32136.1 | 25383 | 28612 | 2114 | | | | | | | | | | |
| 455 | | KXP24174.1 | 25385 | 28614 | 2114 | | | | | | | | | | |
| 456 | | KXR08922.1 | 25387 | 28616 | 2114 | | | | | | | | | | |
| 457 | | KXR32560.1 | 25389 | 28618 | 2114 | | | | | | | | | | |
| 458 | | KXR26310.1 | 25391 | 28620 | 2114 | | | | | | | | | | |
| 459 | | KXR92242.1 | 25393 | 28622 | 2114 | | | | | | | | | | |
| 460 | | KXR64178.1 | 25395 | 28624 | 2114 | | | | | | | | | | |
| 461 | | KXR53387.1 | 25397 | 28626 | 2114 | | | | | | | | | | |
| 462 | | EKJ54998.1 | 25399 | 28628 | 2104 | | | | | | | | | | |
| 463 | | ERC53519.1 | 25401 | 28630 | 2104 | | | | | | | | | | |
| 464 | | ERE00883.1 | 25403 | 28632 | 2104 | | | | | | | | | | |
| 465 | | ERE21799.1 | 25405 | 28634 | 2104 | | | | | | | | | | |
| 466 | | ERB68143.1 | 25407 | 28636 | 2104 | | | | | | | | | | |
| 467 | | ELV15311.1 | 25409 | 28638 | 2104 | | | | | | | | | | |
| 468 | | EKK91185.1 | 25411 | 28640 | 2104 | | | | | | | | | | |
| 469 | | EKK67942.1 | 25413 | 28642 | 2104 | | | | | | | | | | |
| 470 | | EKK62293.1 | 25415 | 28644 | 2104 | | | | | | | | | | |
| 471 | | EYE30769.1 | 25417 | 28646 | 2104 | | | | | | | | | | |
| 472 | | EYE15511.1 | 25419 | 28648 | 2104 | | | | | | | | | | |
| 473 | | EYE15598.1 | 25421 | 28650 | 2104 | | | | | | | | | | |
| 474 | | ESA76409.1 | 25423 | 28652 | 2090 | | | | | | | | | | |
| 475 | | EZJ86908.1 | 25425 | 28654 | 2104 | | | | | | | | | | |
| 476 | | KEO21429.1 | 25427 | 28656 | 2104 | | | | | | | | | | |
| 477 | | KDA70980.1 | 25429 | 28658 | 2104 | | | | | | | | | | |
| 478 | | KDS94627.1 | 25431 | 28660 | 2114 | | | | | | | | | | |
| 479 | | KDT14053.1 | 25433 | 28662 | 2114 | | | | | | | | | | |
| 480 | | KEN70536.1 | 25435 | 28664 | 2114 | | | | | | | | | | |
| 481 | | KDV77036.1 | 25437 | 28666 | 2114 | | | | | | | | | | |
| 482 | | KDW27005.1 | 25439 | 28668 | 2114 | | | | | | | | | | |
| 483 | | KEN84157.1 | 25441 | 28670 | 2114 | | | | | | | | | | |
| 484 | | KEO02789.1 | 25443 | 28672 | 2114 | | | | | | | | | | |
| 485 | | KDX84120.1 | 25445 | 28674 | 2114 | | | | | | | | | | |
| 486 | | KEJ20443.1 | 25447 | 28676 | 2071 | | | | | | | | | | |
| 487 | | KEJ21099.1 | 25449 | 28678 | 2104 | | | | | | | | | | |
| 488 | | EMX91855.1 | 25451 | 28680 | 2657 | | | | | | | | | | |
| 489 | | EMW69586.1 | 25453 | 28682 | 2114 | | | | | | | | | | |
| 490 | | EMW64104.1 | 25455 | 28684 | 2114 | | | | | | | | | | |
| 491 | | EMW45432.1 | 25457 | 28686 | 2114 | | | | | | | | | | |
| 492 | | EMV86399.1 | 25459 | 28688 | 2114 | | | | | | | | | | |
| 493 | | ENA88227.1 | 25461 | 28690 | 2114 | | | | | | | | | | |
| 494 | | ENB03534.1 | 25463 | 28692 | 2090 | | | | | | | | | | |
| 495 | | ERD09266.1 | 25465 | 28694 | 2104 | | | | | | | | | | |
| 496 | | EKK37396.1 | 25467 | 28696 | 2104 | | | | | | | | | | |
| 497 | | ELW28319.1 | 25469 | 28698 | 2104 | | | | | | | | | | |
| 498 | | KDT83303.1 | 25471 | 28700 | 2104 | | | | | | | | | | |
| 499 | | KEK80770.1 | 25473 | 28702 | 2104 | | | | | | | | | | |
| 500 | | KEL07348.1 | 25475 | 28704 | 2104 | | | | | | | | | | |
| 501 | | KEK94607.1 | 25477 | 28706 | 2104 | | | | | | | | | | |
| 502 | | EKK37001.1 | 25479 | 28708 | 2104 | | | | | | | | | | |
| 503 | | EKI06062.1 | 25481 | 28710 | 2104 | | | | | | | | | | |
| 504 | | EIL64995.1 | 25483 | 28712 | 2114 | | | | | | | | | | |
| 505 | | EKI02734.1 | 25485 | 28714 | 2104 | | | | | | | | | | |
| 506 | | KEL97939.1 | 25487 | 28716 | 2104 | | | | | | | | | | |
| 507 | | KEM08515.1 | 25489 | 28718 | 2104 | | | | | | | | | | |
| 508 | | KEM49607.1 | 25491 | 28720 | 2104 | | | | | | | | | | |
| 509 | | KEN01218.1 | 25493 | 28722 | 2114 | | | | | | | | | | |
| 510 | | KEM09157.1 | 25495 | 28724 | 2114 | | | | | | | | | | |
| 511 | | KEM25601.1 | 25497 | 28726 | 2114 | | | | | | | | | | |
| 512 | | KEM85724.1 | 25499 | 28728 | 2104 | | | | | | | | | | |
| 513 | | KEN65096.1 | 25501 | 28730 | 2104 | | | | | | | | | | |
| 514 | | ELW21807.1 | 25503 | 28732 | 2104 | | | | | | | | | | |
| 515 | | EKK71769.1 | 25505 | 28734 | 2104 | | | | | | | | | | |
| 516 | | EKK61091.1 | 25507 | 28736 | 2104 | | | | | | | | | | |
| 517 | | EKK52335.1 | 25509 | 28738 | 2104 | | | | | | | | | | |
| 518 | | EKK87669.1 | 25511 | 28740 | 2104 | | | | | | | | | | |
| 519 | | EKV85101.1 | 25513 | 28742 | 2104 | | | | | | | | | | |
| 520 | | EKV70655.1 | 25515 | 28744 | 2104 | | | | | | | | | | |
| 521 | | EKW05774.1 | 25517 | 28746 | 2104 | | | | | | | | | | |
| 522 | | EKW03229.1 | 25519 | 28748 | 2104 | | | | | | | | | | |
| 523 | | ESD10541.1 | 25521 | 28750 | 2104 | | | | | | | | | | |
| 524 | | ESA83483.1 | 25523 | 28752 | 2090 | | | | | | | | | | |
| 525 | | ESD19201.1 | 25525 | 28754 | 2090 | | | | | | | | | | |
| 526 | | ESD45641.1 | 25527 | 28756 | 2090 | | | | | | | | | | |
| 527 | | ESD61126.1 | 25529 | 28758 | 2090 | | | | | | | | | | |
| 528 | | ESD71191.1 | 25531 | 28760 | 2104 | | | | | | | | | | |
| 529 | | ESD92014.1 | 25533 | 28762 | 2090 | | | | | | | | | | |
| 530 | | ESD92684.1 | 25535 | 28764 | 2090 | | | | | | | | | | |
| 531 | | EKW04204.1 | 25537 | 28766 | 2104 | | | | | | | | | | |
| 532 | | EKW19026.1 | 25539 | 28768 | 2104 | | | | | | | | | | |
| 533 | | EIN33839.1 | 25541 | 28770 | 2104 | | | | | | | | | | |
| 534 | | EKW08489.1 | 25543 | 28772 | 2104 | | | | | | | | | | |
| 535 | | ELW29953.1 | 25545 | 28774 | 2104 | | | | | | | | | | |
| 536 | | EKW22423.1 | 25547 | 28776 | 2104 | | | | | | | | | | |
| 537 | | EKW74828.1 | 25549 | 28778 | 2104 | | | | | | | | | | |
| 538 | | EKY34593.1 | 25551 | 28780 | 2104 | | | | | | | | | | |
| 539 | | EKW38126.1 | 25553 | 28782 | 2104 | | | | | | | | | | |
| 540 | | EKW51346.1 | 25555 | 28784 | 2104 | | | | | | | | | | |
| 541 | | EKW70677.1 | 25557 | 28786 | 2104 | | | | | | | | | | |
| 542 | | EKW68015.1 | 25559 | 28788 | 2104 | | | | | | | | | | |
| 543 | | EKW71373.1 | 25561 | 28790 | 2104 | | | | | | | | | | |
| 544 | | EKY34710.1 | 25563 | 28792 | 2104 | | | | | | | | | | |
| 545 | | EKW75844.1 | 25565 | 28794 | 2104 | | | | | | | | | | |
| 546 | | ELW46373.1 | 25567 | 28796 | 2104 | | | | | | | | | | |
| 547 | | EKW96658.1 | 25569 | 28798 | 2104 | | | | | | | | | | |
| 548 | | EKW96262.1 | 25571 | 28800 | 2104 | | | | | | | | | | |
| 549 | | ELV31252.1 | 25573 | 28802 | 2104 | | | | | | | | | | |
| 550 | | ELV35525.1 | 25575 | 28804 | 2104 | | | | | | | | | | |
| 551 | | ELV44385.1 | 25577 | 28806 | 2104 | | | | | | | | | | |
| 552 | | ELW27709.1 | 25579 | 28808 | 2104 | | | | | | | | | | |
| 553 | | ELV47170.1 | 25581 | 28810 | 2104 | | | | | | | | | | |
| 554 | | ELW24264.1 | 25583 | 28812 | 2104 | | | | | | | | | | |
| 555 | | ELV62894.1 | 25585 | 28814 | 2104 | | | | | | | | | | |
| 556 | | ETJ70882.1 | 25587 | 28816 | 2104 | | | | | | | | | | |
| 557 | | ELV63261.1 | 25589 | 28818 | 2104 | | | | | | | | | | |
| 558 | | ACA79488.1 | 25591 | 28820 | 2104 | | | | | | | | | | |
| 559 | | ETJ79055.1 | 25593 | 28822 | 2104 | | | | | | | | | | |
| 560 | | ERB88976.1 | 25595 | 28824 | 2104 | | | | | | | | | | |
| 561 | | ERD08450.1 | 25597 | 28826 | 2104 | | | | | | | | | | |
| 562 | | ERD05022.1 | 25599 | 28828 | 2104 | | | | | | | | | | |
| 563 | | ERD20749.1 | 25601 | 28830 | 2104 | | | | | | | | | | |
| 564 | | ERD22952.1 | 25603 | 28832 | 2104 | | | | | | | | | | |
| 565 | | ERB79218.1 | 25605 | 28834 | 2104 | | | | | | | | | | |
| 566 | | ERD23993.1 | 25607 | 28836 | 2104 | | | | | | | | | | |
| 567 | | ERD35139.1 | 25609 | 28838 | 2104 | | | | | | | | | | |
| 568 | | ERD37186.1 | 25611 | 28840 | 2104 | | | | | | | | | | |
| 569 | | ERD26785.1 | 25613 | 28842 | 2104 | | | | | | | | | | |
| 570 | | ERD52932.1 | 25615 | 28844 | 2104 | | | | | | | | | | |
| 571 | | ERD40233.1 | 25617 | 28846 | 2104 | | | | | | | | | | |
| 572 | | ERD45130.1 | 25619 | 28848 | 2104 | | | | | | | | | | |
| 573 | | ERB91933.1 | 25621 | 28850 | 2104 | | | | | | | | | | |
| 574 | | ERC00105.1 | 25623 | 28852 | 2104 | | | | | | | | | | |
| 575 | | ERC08313.1 | 25625 | 28854 | 2104 | | | | | | | | | | |
| 576 | | ERC07683.1 | 25627 | 28856 | 2104 | | | | | | | | | | |
| 577 | | ERC15356.1 | 25629 | 28858 | 2104 | | | | | | | | | | |
| 578 | | ERC23447.1 | 25631 | 28860 | 2104 | | | | | | | | | | |
| 579 | | ERC10661.1 | 25633 | 28862 | 2104 | | | | | | | | | | |
| 580 | | ERC32307.1 | 25635 | 28864 | 2104 | | | | | | | | | | |
| 581 | | ERD72600.1 | 25637 | 28866 | 2104 | | | | | | | | | | |
| 582 | | ERD68378.1 | 25639 | 28868 | 2104 | | | | | | | | | | |
| 583 | | ERD73346.1 | 25641 | 28870 | 2104 | | | | | | | | | | |
| 584 | | ERD83190.1 | 25643 | 28872 | 2104 | | | | | | | | | | |
| 585 | | ERC25312.1 | 25645 | 28874 | 2104 | | | | | | | | | | |
| 586 | | ERC30062.1 | 25647 | 28876 | 2104 | | | | | | | | | | |
| 587 | | ERD87992.1 | 25649 | 28878 | 2104 | | | | | | | | | | |
| 588 | | ERD91778.1 | 25651 | 28880 | 2104 | | | | | | | | | | |
| 589 | | ERD98685.1 | 25653 | 28882 | 2104 | | | | | | | | | | |
| 590 | | ERD88430.1 | 25655 | 28884 | 2104 | | | | | | | | | | |
| 591 | | ERE36113.1 | 25657 | 28886 | 2104 | | | | | | | | | | |
| 592 | | ERE23642.1 | 25659 | 28888 | 2104 | | | | | | | | | | |
| 593 | | ERC33936.1 | 25661 | 28890 | 2104 | | | | | | | | | | |
| 594 | | ERC40343.1 | 25663 | 28892 | 2104 | | | | | | | | | | |
| 595 | | ERC48260.1 | 25665 | 28894 | 2104 | | | | | | | | | | |
| 596 | | ETY20411.1 | 25667 | 28896 | 2114 | | | | | | | | | | |
| 597 | | ETY13489.1 | 25669 | 28898 | 2114 | | | | | | | | | | |
| 598 | | ETY13648.1 | 25671 | 28900 | 2114 | | | | | | | | | | |
| 599 | | ETY08682.1 | 25673 | 28902 | 2114 | | | | | | | | | | |
| 600 | | ETX96687.1 | 25675 | 28904 | 2114 | | | | | | | | | | |
| 601 | | ERO99484.1 | 25677 | 28906 | 2114 | | | | | | | | | | |
| 602 | | ETY35402.1 | 25679 | 28908 | 2114 | | | | | | | | | | |
| 603 | | ETY29966.1 | 25681 | 28910 | 2114 | | | | | | | | | | |
| 604 | | ESL20614.1 | 25683 | 28912 | 2104 | | | | | | | | | | |
| 605 | | ETX82126.1 | 25685 | 28914 | 2114 | | | | | | | | | | |
| 606 | | ETX76976.1 | 25687 | 28916 | 2114 | | | | | | | | | | |
| 607 | | ETY61260.1 | 25689 | 28918 | 2114 | | | | | | | | | | |
| 608 | | EZQ66604.1 | 25691 | 28920 | 2114 | | | | | | | | | | |
| 609 | | ETY23955.1 | 25693 | 28922 | 2114 | | | | | | | | | | |
| 610 | | EKH96740.1 | 25695 | 28924 | 2104 | | | | | | | | | | |
| 611 | | CDU33063.1 | 25697 | 28926 | 2114 | | | | | | | | | | |
| 612 | | EHV18501.1 | 25699 | 28928 | 2104 | | | | | | | | | | |
| 613 | | EIP71495.1 | 25701 | 28930 | 2104 | | | | | | | | | | |
| 614 | | EKI46661.1 | 25703 | 28932 | 2104 | | | | | | | | | | |
| 615 | | EKI57136.1 | 25705 | 28934 | 2104 | | | | | | | | | | |
| 616 | | EKI59126.1 | 25707 | 28936 | 2104 | | | | | | | | | | |
| 617 | | EIP63563.1 | 25709 | 28938 | 2104 | | | | | | | | | | |
| 618 | | EIP72315.1 | 25711 | 28940 | 2104 | | | | | | | | | | |
| 619 | | EKI64841.1 | 25713 | 28942 | 2104 | | | | | | | | | | |
| 620 | | EKI72765.1 | 25715 | 28944 | 2104 | | | | | | | | | | |
| 621 | | EKI91872.1 | 25717 | 28946 | 2104 | | | | | | | | | | |
| 622 | | EKI82677.1 | 25719 | 28948 | 2104 | | | | | | | | | | |
| 623 | | EKJ05431.1 | 25721 | 28950 | 2104 | | | | | | | | | | |
| 624 | | EKI92955.1 | 25723 | 28952 | 2104 | | | | | | | | | | |
| 625 | | EKJ00854.1 | 25725 | 28954 | 2104 | | | | | | | | | | |
| 626 | | EIP72928.1 | 25727 | 28956 | 2104 | | | | | | | | | | |
| 627 | | EKJ19827.1 | 25729 | 28958 | 2104 | | | | | | | | | | |
| 628 | | EKJ20864.1 | 25731 | 28960 | 2104 | | | | | | | | | | |
| 629 | | EKJ36038.1 | 25733 | 28962 | 2104 | | | | | | | | | | |
| 630 | | EKJ47665.1 | 25735 | 28964 | 2104 | | | | | | | | | | |
| 631 | | EKJ36501.1 | 25737 | 28966 | 2104 | | | | | | | | | | |
| 632 | | EIP38855.1 | 25739 | 28968 | 2104 | | | | | | | | | | |
| 633 | | EIO92486.1 | 25741 | 28970 | 2104 | | | | | | | | | | |
| 634 | | EIO87517.1 | 25743 | 28972 | 2104 | | | | | | | | | | |
| 635 | | EIP27283.1 | 25745 | 28974 | 2104 | | | | | | | | | | |
| 636 | | EIP10343.1 | 25747 | 28976 | 2104 | | | | | | | | | | |
| 637 | | EIP33944.1 | 25749 | 28978 | 2104 | | | | | | | | | | |
| 638 | | EIP40257.1 | 25751 | 28980 | 2104 | | | | | | | | | | |
| 639 | | EIP48993.1 | 25753 | 28982 | 2104 | | | | | | | | | | |
| 640 | | EIP35357.1 | 25755 | 28984 | 2104 | | | | | | | | | | |
| 641 | | EIP50032.1 | 25757 | 28986 | 2104 | | | | | | | | | | |
| 642 | | EKI03043.1 | 25759 | 28988 | 2104 | | | | | | | | | | |
| 643 | | EST65289.1 | 25761 | 28990 | 2114 | | | | | | | | | | |
| 644 | | EMX76370.1 | 25763 | 28992 | 1715 | | | | | | | | | | |
| 645 | | EMX78116.1 | 25765 | 28994 | 1715 | | | | | | | | | | |
| 646 | | EKH36317.1 | 25767 | 28996 | 2104 | | | | | | | | | | |
| 647 | | EIN15911.1 | 25769 | 28998 | 2104 | | | | | | | | | | |
| 648 | | EKH25288.1 | 25771 | 29000 | 2104 | | | | | | | | | | |
| 649 | | EKH13354.1 | 25773 | 29002 | 2104 | | | | | | | | | | |
| 650 | | EIN16634.1 | 25775 | 29004 | 2104 | | | | | | | | | | |
| 651 | | EIN33026.1 | 25777 | 29006 | 2104 | | | | | | | | | | |
| 652 | | EKH48832.1 | 25779 | 29008 | 2104 | | | | | | | | | | |
| 653 | | EKJ48076.1 | 25781 | 29010 | 2104 | | | | | | | | | | |
| 654 | | EFI22364.1 | 25783 | 29012 | 2114 | | | | | | | | | | |
| 655 | | EFF02931.1 | 25785 | 29014 | 2114 | | | | | | | | | | |
| 656 | | EQN36980.1 | 25787 | 29016 | 2114 | | | | | | | | | | |
| 657 | | EQQ54038.1 | 25789 | 29018 | 2114 | | | | | | | | | | |
| 658 | | EQQ62151.1 | 25791 | 29020 | 1302 | | | | | | | | | | |
| 659 | | EQQ82445.1 | 25793 | 29022 | 2114 | | | | | | | | | | |
| 660 | | EQR15650.1 | 25795 | 29024 | 2114 | | | | | | | | | | |
| 661 | | EQR29834.1 | 25797 | 29026 | 2114 | | | | | | | | | | |
| 662 | | EQR56342.1 | 25799 | 29028 | 2114 | | | | | | | | | | |
| 663 | | EQR70531.1 | 25801 | 29030 | 2114 | | | | | | | | | | |
| 664 | | EQR73388.1 | 25803 | 29032 | 2114 | | | | | | | | | | |
| 665 | | ESP12228.1 | 25805 | 29034 | 2104 | | | | | | | | | | |
| 666 | | EQR98265.1 | 25807 | 29036 | 2114 | | | | | | | | | | |
| 667 | | EQS19620.1 | 25809 | 29038 | 2114 | | | | | | | | | | |
| 668 | | EQS44554.1 | 25811 | 29040 | 2114 | | | | | | | | | | |
| 669 | | EQS43788.1 | 25813 | 29042 | 2114 | | | | | | | | | | |
| 670 | | ERA63201.1 | 25815 | 29044 | 2114 | | | | | | | | | | |
| 671 | | EQS73534.1 | 25817 | 29046 | 2114 | | | | | | | | | | |
| 672 | | EQS83533.1 | 25819 | 29048 | 2114 | | | | | | | | | | |
| 673 | | EQT06519.1 | 25821 | 29050 | 2114 | | | | | | | | | | |
| 674 | | EQT31741.1 | 25823 | 29052 | 2104 | | | | | | | | | | |
| 675 | | EQT33091.1 | 25825 | 29054 | 2114 | | | | | | | | | | |
| 676 | | EQT84560.1 | 25827 | 29056 | 2104 | | | | | | | | | | |
| 677 | | EQU07296.1 | 25829 | 29058 | 2114 | | | | | | | | | | |
| 678 | | EQU46108.1 | 25831 | 29060 | 2114 | | | | | | | | | | |
| 679 | | EQU56271.1 | 25833 | 29062 | 2114 | | | | | | | | | | |
| 680 | | EQU82066.1 | 25835 | 29064 | 2114 | | | | | | | | | | |
| 681 | | EQN74806.1 | 25837 | 29066 | 2114 | | | | | | | | | | |
| 682 | | EQV15999.1 | 25839 | 29068 | 2114 | | | | | | | | | | |
| 683 | | EQO01912.1 | 25841 | 29070 | 2114 | | | | | | | | | | |
| 684 | | EQO57394.1 | 25843 | 29072 | 2104 | | | | | | | | | | |
| 685 | | EQO66405.1 | 25845 | 29074 | 2104 | | | | | | | | | | |
| 686 | | EQO74550.1 | 25847 | 29076 | 2114 | | | | | | | | | | |
| 687 | | EQP20054.1 | 25849 | 29078 | 2114 | | | | | | | | | | |
| 688 | | EQP30926.1 | 25851 | 29080 | 2114 | | | | | | | | | | |
| 689 | | EQP87867.1 | 25853 | 29082 | 2114 | | | | | | | | | | |
| 690 | | EQV39985.1 | 25855 | 29084 | 2114 | | | | | | | | | | |
| 691 | | EQV77954.1 | 25857 | 29086 | 2114 | | | | | | | | | | |
| 692 | | EQV75318.1 | 25859 | 29088 | 2114 | | | | | | | | | | |
| 693 | | EQV93985.1 | 25861 | 29090 | 2114 | | | | | | | | | | |
| 694 | | EOX00083.1 | 25863 | 29092 | 2114 | | | | | | | | | | |
| 695 | | EOW11795.1 | 25865 | 29094 | 2114 | | | | | | | | | | |
| 696 | | ELF04268.1 | 25867 | 29096 | 2114 | | | | | | | | | | |
| 697 | | ELI36532.1 | 25869 | 29098 | 2114 | | | | | | | | | | |
| 698 | | ELI49202.1 | 25871 | 29100 | 2104 | | | | | | | | | | |
| 699 | | EOW43092.1 | 25873 | 29102 | 2114 | | | | | | | | | | |
| 700 | | ELI49522.1 | 25875 | 29104 | 2114 | | | | | | | | | | |
| 701 | | EOW53696.1 | 25877 | 29106 | 2114 | | | | | | | | | | |
| 702 | | EOW53661.1 | 25879 | 29108 | 2114 | | | | | | | | | | |
| 703 | | EOW60866.1 | 25881 | 29110 | 2114 | | | | | | | | | | |
| 704 | | ELG97963.1 | 25883 | 29112 | 2114 | | | | | | | | | | |
| 705 | | ELH03274.1 | 25885 | 29114 | 2114 | | | | | | | | | | |
| 706 | | ELF28434.1 | 25887 | 29116 | 2104 | | | | | | | | | | |
| 707 | | EU30967.1 | 25889 | 29118 | 2114 | | | | | | | | | | |
| 708 | | EOW72675.1 | 25891 | 29120 | 2114 | | | | | | | | | | |
| 709 | | ELC68673.1 | 25893 | 29122 | 2114 | | | | | | | | | | |
| 710 | | ELC04770.1 | 25895 | 29124 | 2114 | | | | | | | | | | |
| 711 | | EOV27243.1 | 25897 | 29126 | 2114 | | | | | | | | | | |
| 712 | | ELH63192.1 | 25899 | 29128 | 2104 | | | | | | | | | | |
| 713 | | ELD00702.1 | 25901 | 29130 | 2114 | | | | | | | | | | |
| 714 | | ELD16727.1 | 25903 | 29132 | 2114 | | | | | | | | | | |
| 715 | | EOV39942.1 | 25905 | 29134 | 2114 | | | | | | | | | | |
| 716 | | EOX13560.1 | 25907 | 29136 | 2114 | | | | | | | | | | |
| 717 | | ELD74209.1 | 25909 | 29138 | 2114 | | | | | | | | | | |
| 718 | | ELC51202.1 | 25911 | 29140 | 2114 | | | | | | | | | | |
| 719 | | ELC65367.1 | 25913 | 29142 | 2114 | | | | | | | | | | |
| 720 | | ELG08157.1 | 25915 | 29144 | 2104 | | | | | | | | | | |
| 721 | | ELE06192.1 | 25917 | 29146 | 1584 | | | | | | | | | | |
| 722 | | ELE13901.1 | 25919 | 29148 | 2114 | | | | | | | | | | |
| 723 | | EOV54810.1 | 25921 | 29150 | 2114 | | | | | | | | | | |
| 724 | | ELE75378.1 | 25923 | 29152 | 2114 | | | | | | | | | | |
| 725 | | ELJ61315.1 | 25925 | 29154 | 2114 | | | | | | | | | | |
| 726 | | ELE76412.1 | 25927 | 29156 | 2114 | | | | | | | | | | |
| 727 | | EGI23327.1 | 25929 | 29158 | 2114 | | | | | | | | | | |
| 728 | | EIF87148.1 | 25931 | 29160 | 2104 | | | | | | | | | | |
| 729 | | KDG67767.1 | 25933 | 29162 | 2114 | | | | | | | | | | |
| 730 | | EFK89793.1 | 25935 | 29164 | 2090 | | | | | | | | | | |
| 731 | | EFK27050.1 | 25937 | 29166 | 2104 | | | | | | | | | | |
| 732 | | EFI89547.1 | 25939 | 29168 | 2114 | | | | | | | | | | |
| 733 | | EFJ73000.1 | 25941 | 29170 | 2090 | | | | | | | | | | |
| 734 | | EKH61308.1 | 25943 | 29172 | 2104 | | | | | | | | | | |
| 735 | | EKJ53063.1 | 25945 | 29174 | 2104 | | | | | | | | | | |
| 736 | | EMS03602.1 | 25947 | 29176 | 2104 | | | | | | | | | | |
| 737 | | KDM81021.1 | 25949 | 29178 | 2104 | | | | | | | | | | |
| 738 | | AHY73976.1 | 25951 | 29180 | 2104 | | | | | | | | | | |
| 739 | | AHG12157.1 | 25953 | 29182 | 2104 | | | | | | | | | | |
| 740 | | EYZ76662.1 | 25955 | 29184 | 2104 | | | | | | | | | | |
| 741 | | EYW98552.1 | 25957 | 29186 | 2104 | | | | | | | | | | |
| 742 | | EYV59037.1 | 25959 | 29188 | 2104 | | | | | | | | | | |
| 743 | | EZE90567.1 | 25961 | 29190 | 2104 | | | | | | | | | | |
| 744 | | EYV46017.1 | 25963 | 29192 | 2104 | | | | | | | | | | |
| 745 | | EYV44258.1 | 25965 | 29194 | 2104 | | | | | | | | | | |
| 746 | | EYV24875.1 | 25967 | 29196 | 2104 | | | | | | | | | | |
| 747 | | EYV22943.1 | 25969 | 29198 | 2104 | | | | | | | | | | |
| 748 | | EYV18168.1 | 25971 | 29200 | 2104 | | | | | | | | | | |
| 749 | | EYV20078.1 | 25973 | 29202 | 2104 | | | | | | | | | | |
| 750 | | EYV16484.1 | 25975 | 29204 | 2104 | | | | | | | | | | |
| 751 | | EYZ14529.1 | 25977 | 29206 | 2104 | | | | | | | | | | |
| 752 | | EZQ47394.1 | 25979 | 29208 | 2104 | | | | | | | | | | |
| 753 | | EZQ50282.1 | 25981 | 29210 | 2104 | | | | | | | | | | |
| 754 | | EFX13464.1 | 25983 | 29212 | 2104 | | | | | | | | | | |
| 755 | | EFX18241.1 | 25985 | 29214 | 2104 | | | | | | | | | | |
| 756 | | KKF77973.1 | 25987 | 29216 | 2104 | | | | | | | | | | |
| 757 | | KKF80397.1 | 25989 | 29218 | 2104 | | | | | | | | | | |
| 758 | | KKY44751.1 | 25991 | 29220 | 2104 | | | | | | | | | | |
| 759 | | ALH93595.1 | 25993 | 29222 | 2104 | | | | | | | | | | |
| 760 | | KRQ04755.1 | 25995 | 29224 | 2104 | | | | | | | | | | |
| 761 | | EYZ52258.1 | 25997 | 29226 | 2104 | | | | | | | | | | |
| 762 | | EYZ38307.1 | 25999 | 29228 | 2104 | | | | | | | | | | |
| 763 | | EYZ37416.1 | 26001 | 29230 | 2104 | | | | | | | | | | |
| 764 | | EYZ36217.1 | 26003 | 29232 | 2104 | | | | | | | | | | |
| 765 | | EYX13628.1 | 26005 | 29234 | 2104 | | | | | | | | | | |
| 766 | | EYX12327.1 | 26007 | 29236 | 2104 | | | | | | | | | | |
| 767 | | EYX02204.1 | 26009 | 29238 | 2104 | | | | | | | | | | |
| 768 | | EZD85330.1 | 26011 | 29240 | 2104 | | | | | | | | | | |
| 769 | | EZE88806.1 | 26013 | 29242 | 2104 | | | | | | | | | | |
| 770 | | EYV70456.1 | 26015 | 29244 | 2104 | | | | | | | | | | |
| 771 | | EZE89449.1 | 26017 | 29246 | 2104 | | | | | | | | | | |
| 772 | | EYV67648.1 | 26019 | 29248 | 2104 | | | | | | | | | | |
| 773 | | EYX70060.1 | 26021 | 29250 | 2104 | | | | | | | | | | |
| 774 | | EYX57443.1 | 26023 | 29252 | 2104 | | | | | | | | | | |
| 775 | | EYX54548.1 | 26025 | 29254 | 2104 | | | | | | | | | | |
| 776 | | EYX48134.1 | 26027 | 29256 | 2104 | | | | | | | | | | |
| 777 | | EYX44881.1 | 26029 | 29258 | 2104 | | | | | | | | | | |
| 778 | | EYX43993.1 | 26031 | 29260 | 2104 | | | | | | | | | | |
| 779 | | EYX33004.1 | 26033 | 29262 | 2104 | | | | | | | | | | |
| 780 | | EYX30696.1 | 26035 | 29264 | 2104 | | | | | | | | | | |
| 781 | | EYX27761.1 | 26037 | 29266 | 2104 | | | | | | | | | | |
| 782 | | EYW85332.1 | 26039 | 29268 | 2104 | | | | | | | | | | |
| 783 | | EYW83365.1 | 26041 | 29270 | 2104 | | | | | | | | | | |
| 784 | | EYW75521.1 | 26043 | 29272 | 2104 | | | | | | | | | | |
| 785 | | EYW71151.1 | 26045 | 29274 | 2104 | | | | | | | | | | |
| 786 | | EYW67179.1 | 26047 | 29276 | 2104 | | | | | | | | | | |
| 787 | | EYW64048.1 | 26049 | 29278 | 2104 | | | | | | | | | | |
| 788 | | EYW57176.1 | 26051 | 29280 | 2104 | | | | | | | | | | |
| 789 | | EYW49142.1 | 26053 | 29282 | 2104 | | | | | | | | | | |
| 790 | | EYW49455.1 | 26055 | 29284 | 2104 | | | | | | | | | | |
| 791 | | EYW43048.1 | 26057 | 29286 | 2104 | | | | | | | | | | |
| 792 | | EYW39162.1 | 26059 | 29288 | 2104 | | | | | | | | | | |
| 793 | | EYW34611.1 | 26061 | 29290 | 2104 | | | | | | | | | | |
| 794 | | EYW25812.1 | 26063 | 29292 | 2104 | | | | | | | | | | |
| 795 | | EYW27182.1 | 26065 | 29294 | 2104 | | | | | | | | | | |
| 796 | | EYW24511.1 | 26067 | 29296 | 2104 | | | | | | | | | | |
| 797 | | EYW09905.1 | 26069 | 29298 | 2104 | | | | | | | | | | |
| 798 | | EYW13502.1 | 26071 | 29300 | 2104 | | | | | | | | | | |
| 799 | | EZF08560.1 | 26073 | 29302 | 2104 | | | | | | | | | | |
| 800 | | EYW07433.1 | 26075 | 29304 | 2104 | | | | | | | | | | |
| 801 | | EZF05481.1 | 26077 | 29306 | 2104 | | | | | | | | | | |
| 802 | | EDZ87478.1 | 26079 | 29308 | 2104 | | | | | | | | | | |
| 803 | | EDZ81928.1 | 26081 | 29310 | 2104 | | | | | | | | | | |
| 804 | | EDU68725.1 | 26083 | 29312 | 2104 | | | | | | | | | | |
| 805 | | EDU54295.1 | 26085 | 29314 | 2104 | | | | | | | | | | |
| 806 | | ACI39430.1 | 26087 | 29316 | 2104 | | | | | | | | | | |
| 807 | | EDU34122.1 | 26089 | 29318 | 2104 | | | | | | | | | | |
| 808 | | EDZ76631.1 | 26091 | 29320 | 2104 | | | | | | | | | | |
| 809 | | EDU76303.1 | 26093 | 29322 | 2104 | | | | | | | | | | |
| 810 | | EDU82569.1 | 26095 | 29324 | 2104 | | | | | | | | | | |
| 811 | | EDU87409.1 | 26097 | 29326 | 2104 | | | | | | | | | | |
| 812 | | EDU98012.1 | 26099 | 29328 | 2104 | | | | | | | | | | |
| 813 | | AAG59484.1 | 26101 | 29330 | 2104 | | | | | | | | | | |
| 814 | | AIG71774.1 | 26103 | 29332 | 2104 | | | | | | | | | | |
| 815 | | EZA89751.1 | 26105 | 29334 | 2104 | | | | | | | | | | |
| 816 | | EZB05974.1 | 26107 | 29336 | 2104 | | | | | | | | | | |
| 817 | | EZB04403.1 | 26109 | 29338 | 2104 | | | | | | | | | | |
| 818 | | EZB11565.1 | 26111 | 29340 | 2104 | | | | | | | | | | |
| 819 | | EYV89285.1 | 26113 | 29342 | 2104 | | | | | | | | | | |
| 820 | | EZB20620.1 | 26115 | 29344 | 2104 | | | | | | | | | | |
| 821 | | EZB18143.1 | 26117 | 29346 | 2104 | | | | | | | | | | |
| 822 | | EZB30349.1 | 26119 | 29348 | 2104 | | | | | | | | | | |
| 823 | | EFX08675.1 | 26121 | 29350 | 2104 | | | | | | | | | | |
| 824 | | EZB32015.1 | 26123 | 29352 | 2104 | | | | | | | | | | |
| 825 | | EZB41524.1 | 26125 | 29354 | 2104 | | | | | | | | | | |
| 826 | | EZB46985.1 | 26127 | 29356 | 2104 | | | | | | | | | | |
| 827 | | EZB48638.1 | 26129 | 29358 | 2104 | | | | | | | | | | |
| 828 | | EZB48354.1 | 26131 | 29360 | 2104 | | | | | | | | | | |
| 829 | | EZB52648.1 | 26133 | 29362 | 2104 | | | | | | | | | | |
| 830 | | EZB74690.1 | 26135 | 29364 | 2104 | | | | | | | | | | |
| 831 | | EZB73674.1 | 26137 | 29366 | 2104 | | | | | | | | | | |
| 832 | | EZB87911.1 | 26139 | 29368 | 2104 | | | | | | | | | | |
| 833 | | EZB80135.1 | 26141 | 29370 | 2104 | | | | | | | | | | |
| 834 | | EZB81695.1 | 26143 | 29372 | 2104 | | | | | | | | | | |
| 835 | | EZB95442.1 | 26145 | 29374 | 2104 | | | | | | | | | | |
| 836 | | EZC17285.1 | 26147 | 29376 | 2104 | | | | | | | | | | |
| 837 | | EZC37645.1 | 26149 | 29378 | 2104 | | | | | | | | | | |
| 838 | | EZC28524.1 | 26151 | 29380 | 2104 | | | | | | | | | | |
| 839 | | EZC43983.1 | 26153 | 29382 | 2104 | | | | | | | | | | |
| 840 | | EZC48748.1 | 26155 | 29384 | 2104 | | | | | | | | | | |
| 841 | | EZC62955.1 | 26157 | 29386 | 2104 | | | | | | | | | | |
| 842 | | EZC70422.1 | 26159 | 29388 | 2104 | | | | | | | | | | |
| 843 | | EZC77662.1 | 26161 | 29390 | 2104 | | | | | | | | | | |
| 844 | | EZC64795.1 | 26163 | 29392 | 2104 | | | | | | | | | | |
| 845 | | EZC83414.1 | 26165 | 29394 | 2104 | | | | | | | | | | |
| 846 | | EZC90333.1 | 26167 | 29396 | 2104 | | | | | | | | | | |
| 847 | | EZC92771.1 | 26169 | 29398 | 2104 | | | | | | | | | | |
| 848 | | EZC86953.1 | 26171 | 29400 | 2104 | | | | | | | | | | |
| 849 | | EZD00267.1 | 26173 | 29402 | 2104 | | | | | | | | | | |
| 850 | | EYV84723.1 | 26175 | 29404 | 2104 | | | | | | | | | | |
| 851 | | EZD08968.1 | 26177 | 29406 | 2104 | | | | | | | | | | |
| 852 | | EZD17519.1 | 26179 | 29408 | 2104 | | | | | | | | | | |
| 853 | | EZD15757.1 | 26181 | 29410 | 2104 | | | | | | | | | | |
| 854 | | EZD19488.1 | 26183 | 29412 | 2104 | | | | | | | | | | |
| 855 | | EZD75269.1 | 26185 | 29414 | 2104 | | | | | | | | | | |
| 856 | | BAB38683.1 | 26187 | 29416 | 2104 | | | | | | | | | | |
| 857 | | AIF96934.1 | 26189 | 29418 | 2104 | | | | | | | | | | |
| 858 | | AJA29436.1 | 26191 | 29420 | 2104 | | | | | | | | | | |
| 859 | | ACT75064.1 | 26193 | 29422 | 2104 | | | | | | | | | | |
| 860 | | EEC30693.1 | 26195 | 29424 | 2104 | | | | | | | | | | |
| 861 | | EYZ63880.1 | 26197 | 29426 | 2104 | | | | | | | | | | |
| 862 | | EFX22734.1 | 26199 | 29428 | 2104 | | | | | | | | | | |
| 863 | | ADD59519.1 | 26201 | 29430 | 2104 | | | | | | | | | | |
| 864 | | ENC26729.1 | 26203 | 29432 | 2114 | | | | | | | | | | |
| 865 | | ENA10896.1 | 26205 | 29434 | 2114 | | | | | | | | | | |
| 866 | | EMZ79273.1 | 26207 | 29436 | 2114 | | | | | | | | | | |
| 867 | | ENG24753.1 | 26209 | 29438 | 2114 | | | | | | | | | | |
| 868 | | ENG35039.1 | 26211 | 29440 | 2114 | | | | | | | | | | |
| 869 | | ENG38631.1 | 26213 | 29442 | 2090 | | | | | | | | | | |
| 870 | | END33529.1 | 26215 | 29444 | 2090 | | | | | | | | | | |
| 871 | | ENE04446.1 | 26217 | 29446 | 2114 | | | | | | | | | | |
| 872 | | ENG47569.1 | 26219 | 29448 | 2090 | | | | | | | | | | |
| 873 | | ENG50954.1 | 26221 | 29450 | 2114 | | | | | | | | | | |
| 874 | | ENG56305.1 | 26223 | 29452 | 2114 | | | | | | | | | | |
| 875 | | ENG57264.1 | 26225 | 29454 | 2090 | | | | | | | | | | |
| 876 | | ENH40674.1 | 26227 | 29456 | 2090 | | | | | | | | | | |
| 877 | | ENG66730.1 | 26229 | 29458 | 2114 | | | | | | | | | | |
| 878 | | ENG74277.1 | 26231 | 29460 | 2114 | | | | | | | | | | |
| 879 | | EST67460.1 | 26233 | 29462 | 2104 | | | | | | | | | | |
| 880 | | EST67374.1 | 26235 | 29464 | 2104 | | | | | | | | | | |
| 881 | | EIN65416.1 | 26237 | 29466 | 2104 | | | | | | | | | | |
| 882 | | ELV75969.1 | 26239 | 29468 | 2104 | | | | | | | | | | |
| 883 | | ELV90964.1 | 26241 | 29470 | 2104 | | | | | | | | | | |
| 884 | | EIN69805.1 | 26243 | 29472 | 2104 | | | | | | | | | | |
| 885 | | EIN85485.1 | 26245 | 29474 | 2104 | | | | | | | | | | |
| 886 | | ELV76555.1 | 26247 | 29476 | 2104 | | | | | | | | | | |
| 887 | | ELV98807.1 | 26249 | 29478 | 2104 | | | | | | | | | | |
| 888 | | EIN94452.1 | 26251 | 29480 | 2104 | | | | | | | | | | |
| 889 | | EKH63851.1 | 26253 | 29482 | 2104 | | | | | | | | | | |
| 890 | | EIN88182.1 | 26255 | 29484 | 2104 | | | | | | | | | | |
| 891 | | EIN89808.1 | 26257 | 29486 | 2104 | | | | | | | | | | |
| 892 | | EIO09765.1 | 26259 | 29488 | 2104 | | | | | | | | | | |
| 893 | | EIN49114.1 | 26261 | 29490 | 2104 | | | | | | | | | | |
| 894 | | EIO07035.1 | 26263 | 29492 | 2104 | | | | | | | | | | |
| 895 | | EIO07406.1 | 26265 | 29494 | 2104 | | | | | | | | | | |
| 896 | | EIO11195.1 | 26267 | 29496 | 2104 | | | | | | | | | | |
| 897 | | EKH20919.1 | 26269 | 29498 | 2104 | | | | | | | | | | |
| 898 | | ELW23234.1 | 26271 | 29500 | 2104 | | | | | | | | | | |
| 899 | | EKI33366.1 | 26273 | 29502 | 2104 | | | | | | | | | | |
| 900 | | EIO24463.1 | 26275 | 29504 | 2104 | | | | | | | | | | |
| 901 | | EKH71757.1 | 26277 | 29506 | 2104 | | | | | | | | | | |
| 902 | | EIO49135.1 | 26279 | 29508 | 2104 | | | | | | | | | | |
| 903 | | EKH87903.1 | 26281 | 29510 | 2104 | | | | | | | | | | |
| 904 | | ELV91644.1 | 26283 | 29512 | 2104 | | | | | | | | | | |
| 905 | | ELV93152.1 | 26285 | 29514 | 2104 | | | | | | | | | | |
| 906 | | EKH83479.1 | 26287 | 29516 | 2104 | | | | | | | | | | |
| 907 | | EIN52368.1 | 26289 | 29518 | 2104 | | | | | | | | | | |
| 908 | | EKH07683.1 | 26291 | 29520 | 2104 | | | | | | | | | | |
| 909 | | ELW12426.1 | 26293 | 29522 | 2104 | | | | | | | | | | |
| 910 | | EIN68648.1 | 26295 | 29524 | 2104 | | | | | | | | | | |
| 911 | | ACB19214.1 | 26297 | 29526 | 2104 | | | | | | | | | | |
| 912 | | ERE14330.1 | 26299 | 29528 | 2104 | | | | | | | | | | |
| 913 | | ERC65825.1 | 26301 | 29530 | 2104 | | | | | | | | | | |
| 914 | | ERC74189.1 | 26303 | 29532 | 2104 | | | | | | | | | | |
| 915 | | ERC92370.1 | 26305 | 29534 | 2104 | | | | | | | | | | |
| 916 | | EGI28995.1 | 26307 | 29536 | 2104 | | | | | | | | | | |
| 917 | | EKH95568.1 | 26309 | 29538 | 2104 | | | | | | | | | | |
| 918 | | EIO46248.1 | 26311 | 29540 | 2104 | | | | | | | | | | |
| 919 | | EIO79506.1 | 26313 | 29542 | 2104 | | | | | | | | | | |
| 920 | | EIO65789.1 | 26315 | 29544 | 2106 | | | | | | | | | | |
| 921 | | EIO79103.1 | 26317 | 29546 | 2104 | | | | | | | | | | |
| 922 | | EIO63560.1 | 26319 | 29548 | 2104 | | | | | | | | | | |
| 923 | | EIO69660.1 | 26321 | 29550 | 2104 | | | | | | | | | | |
| 924 | | EIP05657.1 | 26323 | 29552 | 2104 | | | | | | | | | | |
| 925 | | EIP03388.1 | 26325 | 29554 | 2104 | | | | | | | | | | |
| 926 | | ERE42063.1 | 26327 | 29556 | 2104 | | | | | | | | | | |
| 927 | | ERE45411.1 | 26329 | 29558 | 2104 | | | | | | | | | | |
| 928 | | KDG81462.1 | 26331 | 29560 | 2114 | | | | | | | | | | |
| 929 | | KDG84325.1 | 26333 | 29562 | 2114 | | | | | | | | | | |
| 930 | | ERB08806.1 | 26335 | 29564 | 2104 | | | | | | | | | | |
| 931 | | ERB13350.1 | 26337 | 29566 | 2104 | | | | | | | | | | |
| 932 | | EQX93205.1 | 26339 | 29568 | 2114 | | | | | | | | | | |
| 933 | | EQY50266.1 | 26341 | 29570 | 2114 | | | | | | | | | | |
| 934 | | ERB23701.1 | 26343 | 29572 | 2114 | | | | | | | | | | |
| 935 | | EQY93867.1 | 26345 | 29574 | 2114 | | | | | | | | | | |
| 936 | | EQZ08532.1 | 26347 | 29576 | 2114 | | | | | | | | | | |
| 937 | | EQZ94053.1 | 26349 | 29578 | 2114 | | | | | | | | | | |
| 938 | | ERA40555.1 | 26351 | 29580 | 2114 | | | | | | | | | | |
| 939 | | CAR16064.1 | 26353 | 29582 | 2114 | | | | | | | | | | |
| 940 | | AFJ31995.1 | 26355 | 29584 | 2104 | | | | | | | | | | |
| 941 | | KWW01508.1 | 26357 | 29586 | 2113 | | | | | | | | | | |
| 942 | | CAQ90578.1 | 26359 | 29588 | 2113 | | | | | | | | | | |
| 943 | | EGC09238.1 | 26361 | 29590 | 2114 | | | | | | | | | | |
| 944 | | EOW71262.1 | 26363 | 29592 | 2114 | | | | | | | | | | |
| 945 | | AJY69764.1 | 26365 | 29594 | 2084 | | | | | | | | | | |
| 946 | | EDL62312.1 | 26367 | 29596 | 2084 | | | | | | | | | | |
| 947 | | EOD77322.1 | 26369 | 29598 | 2144 | | | | | | | | | | |
| 948 | | KJS14046.1 | 26371 | 29600 | 2046 | | | | | | | | | | |
| 949 | | EAQ32828.1 | 26373 | 29602 | 2099 | | | | | | | | | | |
| 950 | | KXS34467.1 | 26375 | 29604 | 2099 | | | | | | | | | | |
| 951 | | CDG80809.1 | 26377 | 29606 | 2106 | | | | | | | | | | |
| 952 | | KMK41965.1 | 26379 | 29608 | 2106 | | | | | | | | | | |
| 953 | | KMV95474.1 | 26381 | 29610 | 2108 | | | | | | | | | | |
| 954 | | EYT05883.1 | 26383 | 29612 | 2108 | | | | | | | | | | |
| 955 | | KGK50933.1 | 26385 | 29614 | 2106 | | | | | | | | | | |
| 956 | | KHE28071.1 | 26387 | 29616 | 2106 | | | | | | | | | | |
| 957 | | KHF57366.1 | 26389 | 29618 | 2106 | | | | | | | | | | |
| 958 | | KHQ22239.1 | 26391 | 29620 | 2106 | | | | | | | | | | |
| 959 | | KLZ78427.1 | 26393 | 29622 | 2106 | | | | | | | | | | |
| 960 | | KMB09547.1 | 26395 | 29624 | 2106 | | | | | | | | | | |
| 961 | | KMG65749.1 | 26397 | 29626 | 2108 | | | | | | | | | | |
| 962 | | KMG69376.1 | 26399 | 29628 | 2106 | | | | | | | | | | |
| 963 | | KMH23798.1 | 26401 | 29630 | 2106 | | | | | | | | | | |
| 964 | | KMH84528.1 | 26403 | 29632 | 2106 | | | | | | | | | | |
| 965 | | KMX40524.1 | 26405 | 29634 | 2106 | | | | | | | | | | |
| 966 | | ALH86088.1 | 26407 | 29636 | 2106 | | | | | | | | | | |
| 967 | | KRR31027.1 | 26409 | 29638 | 2108 | | | | | | | | | | |
| 968 | | ALQ84490.1 | 26411 | 29640 | 2106 | | | | | | | | | | |
| 969 | | ALQ89985.1 | 26413 | 29642 | 2106 | | | | | | | | | | |
| 970 | | KSX16169.1 | 26415 | 29644 | 2106 | | | | | | | | | | |
| 971 | | KSX18601.1 | 26417 | 29646 | 2106 | | | | | | | | | | |
| 972 | | KSX52402.1 | 26419 | 29648 | 2106 | | | | | | | | | | |
| 973 | | ALP76744.1 | 26421 | 29650 | 2106 | | | | | | | | | | |
| 974 | | KSZ32505.1 | 26423 | 29652 | 2106 | | | | | | | | | | |
| 975 | | KUF65064.1 | 26425 | 29654 | 2106 | | | | | | | | | | |
| 976 | | KUG47427.1 | 26427 | 29656 | 2106 | | | | | | | | | | |
| 977 | | KXO23872.1 | 26429 | 29658 | 2106 | | | | | | | | | | |
| 978 | | KYM26278.1 | 26431 | 29660 | 2106 | | | | | | | | | | |
| 979 | | ERN57701.1 | 26433 | 29662 | 2082 | | | | | | | | | | |
| 980 | | EWE85118.1 | 26435 | 29664 | 2106 | | | | | | | | | | |
| 981 | | ESL33173.1 | 26437 | 29666 | 2106 | | | | | | | | | | |
| 982 | | KDK59886.1 | 26439 | 29668 | 2106 | | | | | | | | | | |
| 983 | | CDK69438.1 | 26441 | 29670 | 2114 | | | | | | | | | | |
| 984 | | EMH91682.1 | 26443 | 29672 | 2106 | | | | | | | | | | |
| 985 | | EOZ63311.1 | 26445 | 29674 | 2082 | | | | | | | | | | |
| 986 | | EOY66995.1 | 26447 | 29676 | 2106 | | | | | | | | | | |
| 987 | | ESN46753.1 | 26449 | 29678 | 2108 | | | | | | | | | | |
| 988 | | EWF66266.1 | 26451 | 29680 | 2106 | | | | | | | | | | |
| 989 | | ESM58757.1 | 26453 | 29682 | 2106 | | | | | | | | | | |
| 990 | | KDL89512.1 | 26455 | 29684 | 2108 | | | | | | | | | | |
| 991 | | AIX83032.1 | 26457 | 29686 | 2106 | | | | | | | | | | |
| 992 | | AIG86151.1 | 26459 | 29688 | 2082 | | | | | | | | | | |
| 993 | | EKB81085.1 | 26461 | 29690 | 2106 | | | | | | | | | | |
| 994 | | ESM10101.1 | 26463 | 29692 | 2106 | | | | | | | | | | |
| 995 | | EPO98451.1 | 26465 | 29694 | 2106 | | | | | | | | | | |
| 996 | | EPA89958.1 | 26467 | 29696 | 2106 | | | | | | | | | | |
| 997 | | EJI89219.1 | 26469 | 29698 | 2103 | | | | | | | | | | |
| 998 | | KDE37040.1 | 26471 | 29700 | 2103 | | | | | | | | | | |
| 999 | | CAH16734.1 | 26473 | 29702 | 2106 | | | | | | | | | | |
| 1000 | | KTD65146.1 | 26475 | 29704 | 2108 | | | | | | | | | | |
| 1001 | | ETX11506.1 | 26477 | 29706 | 2097 | | | | | | | | | | |
| 1002 | | ESY80708.1 | 26479 | 29708 | 2007 | | | | | | | | | | |
| 1003 | | ESY66205.1 | 26481 | 29710 | 2034 | | | | | | | | | | |
| 1004 | | ESY44731.1 | 26483 | 29712 | 2037 | | | | | | | | | | |
| 1005 | | KQU80074.1 | 26485 | 29714 | 2042 | | | | | | | | | | |
| 1006 | | EMS40059.1 | 26487 | 29716 | 2080 | | | | | | | | | | |
| 1007 | | EMS40205.1 | 26489 | 29718 | 2080 | | | | | | | | | | |
| 1008 | | KQQ15550.1 | 26491 | 29720 | 2079 | | | | | | | | | | |
| 1009 | | KQO59081.1 | 26493 | 29722 | 2002 | | | | | | | | | | |
| 1010 | | KQO90918.1 | 26495 | 29724 | 2035 | | | | | | | | | | |
| 1011 | | EFV81023.1 | 26497 | 29726 | 1694 | | | | | | | | | | |
| 1012 | | EHJ60591.1 | 26499 | 29728 | 1959 | | | | | | | | | | |
| 1013 | | AIT79632.1 | 26501 | 29730 | 1987 | | | | | | | | | | |
| 1014 | | CCA92392.1 | 26503 | 29732 | 1985 | | | | | | | | | | |
| 1015 | | KTS15500.1 | 26505 | 29734 | 2108 | | | | | | | | | | |
| 1016 | | KTS86096.1 | 26507 | 29736 | 2108 | | | | | | | | | | |
| 1017 | | EFM21232.1 | 26509 | 29738 | 2109 | | | | | | | | | | |
| 1018 | | KGA41246.1 | 26511 | 29740 | 2104 | | | | | | | | | | |
| 1019 | | ACX87465.1 | 26513 | 29742 | 2104 | | | | | | | | | | |
| 1020 | | EAU47586.1 | 26515 | 29744 | 2003 | | | | | | | | | | |
| 1021 | | EAS45763.1 | 26517 | 29746 | 2065 | | | | | | | | | | |
| 1022 | | KJM66695.1 | 26519 | 29748 | 2105 | | | | | | | | | | |
| 1023 | | KMK11833.1 | 26521 | 29750 | 2105 | | | | | | | | | | |
| 1024 | | KMK24779.1 | 26523 | 29752 | 2105 | | | | | | | | | | |
| 1025 | | KXU56724.1 | 26525 | 29754 | 2024 | | | | | | | | | | |
| 1026 | | EGW47202.1 | 26527 | 29756 | 2027 | | | | | | | | | | |
| 1027 | | KSX98964.1 | 26529 | 29758 | 2094 | | | | | | | | | | |
| 1028 | | KTF11893.1 | 26531 | 29760 | 2100 | | | | | | | | | | |
| 1029 | | KGB84628.1 | 26533 | 29762 | 2102 | | | | | | | | | | |
| 1030 | | KHE32029.1 | 26535 | 29764 | 2071 | | | | | | | | | | |
| 1031 | | KQJ75582.1 | 26537 | 29766 | 2111 | | | | | | | | | | |
| 1032 | | KRV15964.1 | 26539 | 29768 | 2071 | | | | | | | | | | |
| 1033 | | KSD09758.1 | 26541 | 29770 | 2111 | | | | | | | | | | |
| 1034 | | KSD19533.1 | 26543 | 29772 | 2071 | | | | | | | | | | |
| 1035 | | KSG03215.1 | 26545 | 29774 | 2071 | | | | | | | | | | |
| 1036 | | KSG67481.1 | 26547 | 29776 | 2111 | | | | | | | | | | |
| 1037 | | KSE90523.1 | 26549 | 29778 | 2065 | | | | | | | | | | |
| 1038 | | KSF67271.1 | 26551 | 29780 | 2111 | | | | | | | | | | |
| 1039 | | KSG19233.1 | 26553 | 29782 | 2071 | | | | | | | | | | |
| 1040 | | KSG75230.1 | 26555 | 29784 | 2071 | | | | | | | | | | |
| 1041 | | KSG85551.1 | 26557 | 29786 | 2071 | | | | | | | | | | |
| 1042 | | KSH39435.1 | 26559 | 29788 | 2071 | | | | | | | | | | |
| 1043 | | KSH60318.1 | 26561 | 29790 | 2071 | | | | | | | | | | |
| 1044 | | KSI21888.1 | 26563 | 29792 | 2071 | | | | | | | | | | |
| 1045 | | KSI51647.1 | 26565 | 29794 | 2111 | | | | | | | | | | |
| 1046 | | KSI82065.1 | 26567 | 29796 | 2111 | | | | | | | | | | |
| 1047 | | KSI99534.1 | 26569 | 29798 | 2071 | | | | | | | | | | |
| 1048 | | KSJ34446.1 | 26571 | 29800 | 2111 | | | | | | | | | | |
| 1049 | | KSK63577.1 | 26573 | 29802 | 2111 | | | | | | | | | | |
| 1050 | | KSL53780.1 | 26575 | 29804 | 2071 | | | | | | | | | | |
| 1051 | | KSM73159.1 | 26577 | 29806 | 2111 | | | | | | | | | | |
| 1052 | | KSM79529.1 | 26579 | 29808 | 2071 | | | | | | | | | | |
| 1053 | | KSN72433.1 | 26581 | 29810 | 2071 | | | | | | | | | | |
| 1054 | | KSO70043.1 | 26583 | 29812 | 2065 | | | | | | | | | | |
| 1055 | | KSQ54928.1 | 26585 | 29814 | 2071 | | | | | | | | | | |
| 1056 | | KSQ13665.1 | 26587 | 29816 | 2071 | | | | | | | | | | |
| 1057 | | KSK33061.1 | 26589 | 29818 | 2071 | | | | | | | | | | |
| 1058 | | KSL80809.1 | 26591 | 29820 | 2071 | | | | | | | | | | |
| 1059 | | KSL64950.1 | 26593 | 29822 | 2071 | | | | | | | | | | |
| 1060 | | KSM29110.1 | 26595 | 29824 | 2111 | | | | | | | | | | |
| 1061 | | KSN07702.1 | 26597 | 29826 | 2111 | | | | | | | | | | |
| 1062 | | KSN22462.1 | 26599 | 29828 | 2111 | | | | | | | | | | |
| 1063 | | KSN45602.1 | 26601 | 29830 | 2071 | | | | | | | | | | |
| 1064 | | KSO16559.1 | 26603 | 29832 | 2111 | | | | | | | | | | |
| 1065 | | KSO84740.1 | 26605 | 29834 | 2071 | | | | | | | | | | |
| 1066 | | KSO99160.1 | 26607 | 29836 | 2111 | | | | | | | | | | |
| 1067 | | KSQ29773.1 | 26609 | 29838 | 2071 | | | | | | | | | | |
| 1068 | | KSR12008.1 | 26611 | 29840 | 2071 | | | | | | | | | | |
| 1069 | | KSR96663.1 | 26613 | 29842 | 2063 | | | | | | | | | | |
| 1070 | | KSS25407.1 | 26615 | 29844 | 2066 | | | | | | | | | | |
| 1071 | | KSS39723.1 | 26617 | 29846 | 2102 | | | | | | | | | | |
| 1072 | | KSR72291.1 | 26619 | 29848 | 2071 | | | | | | | | | | |
| 1073 | | KSS45916.1 | 26621 | 29850 | 2111 | | | | | | | | | | |
| 1074 | | ALY37667.1 | 26623 | 29852 | 2111 | | | | | | | | | | |
| 1075 | | ALY62761.1 | 26625 | 29854 | 2111 | | | | | | | | | | |
| 1076 | | KXC98055.1 | 26627 | 29856 | 2071 | | | | | | | | | | |
| 1077 | | KXC86270.1 | 26629 | 29858 | 2071 | | | | | | | | | | |
| 1078 | | KXD25935.1 | 26631 | 29860 | 2071 | | | | | | | | | | |
| 1079 | | KXD08976.1 | 26633 | 29862 | 2071 | | | | | | | | | | |
| 1080 | | KXD14400.1 | 26635 | 29864 | 2071 | | | | | | | | | | |
| 1081 | | KXD23256.1 | 26637 | 29866 | 2071 | | | | | | | | | | |
| 1082 | | KXC87701.1 | 26639 | 29868 | 2071 | | | | | | | | | | |
| 1083 | | KXF38181.1 | 26641 | 29870 | 2071 | | | | | | | | | | |
| 1084 | | KXF47395.1 | 26643 | 29872 | 2071 | | | | | | | | | | |
| 1085 | | KXF43344.1 | 26645 | 29874 | 2071 | | | | | | | | | | |
| 1086 | | KYO95528.1 | 26647 | 29876 | 2111 | | | | | | | | | | |
| 1087 | | KAJ18077.1 | 26649 | 29878 | 2071 | | | | | | | | | | |
| 1088 | | ERY57593.1 | 26651 | 29880 | 2071 | | | | | | | | | | |
| 1089 | | ERY65607.1 | 26653 | 29882 | 2071 | | | | | | | | | | |
| 1090 | | ERV59835.1 | 26655 | 29884 | 2071 | | | | | | | | | | |
| 1091 | | ERY33374.1 | 26657 | 29886 | 2071 | | | | | | | | | | |
| 1092 | | ERY37451.1 | 26659 | 29888 | 2071 | | | | | | | | | | |
| 1093 | | ERY30829.1 | 26661 | 29890 | 2071 | | | | | | | | | | |
| 1094 | | ERW68206.1 | 26663 | 29892 | 2071 | | | | | | | | | | |
| 1095 | | ERW13882.1 | 26665 | 29894 | 2071 | | | | | | | | | | |
| 1096 | | ERV79732.1 | 26667 | 29896 | 2071 | | | | | | | | | | |
| 1097 | | ETV52349.1 | 26669 | 29898 | 1470 | | | | | | | | | | |
| 1098 | | ERU88468.1 | 26671 | 29900 | 2071 | | | | | | | | | | |
| 1099 | | ERU86603.1 | 26673 | 29902 | 2071 | | | | | | | | | | |
| 1100 | | KEA15335.1 | 26675 | 29904 | 2071 | | | | | | | | | | |
| 1101 | | ERZ32986.1 | 26677 | 29906 | 2071 | | | | | | | | | | |
| 1102 | | GAA19670.1 | 26679 | 29908 | 2071 | | | | | | | | | | |
| 1103 | | EME92221.1 | 26681 | 29910 | 2111 | | | | | | | | | | |
| 1104 | | EYU00121.1 | 26683 | 29912 | 2111 | | | | | | | | | | |
| 1105 | | EOT13873.1 | 26685 | 29914 | 2066 | | | | | | | | | | |
| 1106 | | ETU84934.1 | 26687 | 29916 | 2068 | | | | | | | | | | |
| 1107 | | ERX16723.1 | 26689 | 29918 | 2071 | | | | | | | | | | |
| 1108 | | EWH25347.1 | 26691 | 29920 | 2071 | | | | | | | | | | |
| 1109 | | GAD64771.1 | 26693 | 29922 | 2066 | | | | | | | | | | |
| 1110 | | GAD61323.1 | 26695 | 29924 | 2066 | | | | | | | | | | |
| 1111 | | EZQ18206.1 | 26697 | 29926 | 2105 | | | | | | | | | | |
| 1112 | | AMO73922.1 | 26699 | 29928 | 2112 | | | | | | | | | | |
| 1113 | | KPW38759.1 | 26701 | 29930 | 2073 | | | | | | | | | | |
| 1114 | | KQB53691.1 | 26703 | 29932 | 2111 | | | | | | | | | | |
| 1115 | | EZI30291.1 | 26705 | 29934 | 2087 | | | | | | | | | | |
| 1116 | | KIR22316.1 | 26707 | 29936 | 2085 | | | | | | | | | | |
| 1117 | | KJH86793.1 | 26709 | 29938 | 2065 | | | | | | | | | | |
| 1118 | | KWV84209.1 | 26711 | 29940 | 2065 | | | | | | | | | | |
| 1119 | | EFQ63350.1 | 26713 | 29942 | 2099 | | | | | | | | | | |
| 1120 | | KMM97951.1 | 26715 | 29944 | 2111 | | | | | | | | | | |
| 1121 | | ENY78086.1 | 26717 | 29946 | 2084 | | | | | | | | | | |
| 1122 | | ENA27917.1 | 26719 | 29948 | 1970 | | | | | | | | | | |
| 1123 | | EWC41755.1 | 26721 | 29950 | 1771 | | | | | | | | | | |
| 1124 | | EMD98966.1 | 26723 | 29952 | 2068 | | | | | | | | | | |
| 1125 | | KPB88882.1 | 26725 | 29954 | 1448 | | | | | | | | | | |
| 1126 | | KFD13679.1 | 26727 | 29956 | 2106 | | | | | | | | | | |
| 1127 | | ACE91184.1 | 26729 | 29958 | 2041 | | | | | | | | | | |
| 1128 | | KKZ86596.1 | 26731 | 29960 | 2036 | | | | | | | | | | |
| 1129 | | KQU04981.1 | 26733 | 29962 | 2042 | | | | | | | | | | |
| 1130 | | CCF21881.1 | 26735 | 29964 | 1778 | | | | | | | | | | |
| 1131 | | KQZ63426.1 | 26737 | 29966 | 2007 | | | | | | | | | | |
| 1132 | | KIV64827.1 | 26739 | 29968 | 2033 | | | | | | | | | | |
| 1133 | | KQB14222.1 | 26741 | 29970 | 2012 | | | | | | | | | | |
| 1134 | | KQB14197.1 | 26743 | 29972 | 2012 | | | | | | | | | | |
| 1135 | | ETX15683.1 | 26745 | 29974 | 2020 | | | | | | | | | | |
| 1136 | | KJS41398.1 | 26747 | 29976 | 1995 | | | | | | | | | | |
| 1137 | | EPX84387.1 | 26749 | 29978 | 1417 | | | | | | | | | | |
| 1138 | | GAS73920.1 | 26751 | 29980 | 2106 | | | | | | | | | | |
| 1139 | | GAS79508.1 | 26753 | 29982 | 2106 | | | | | | | | | | |
| 1140 | | KHP17885.1 | 26755 | 29984 | 2106 | | | | | | | | | | |
| 1141 | | KHP06994.1 | 26757 | 29986 | 2106 | | | | | | | | | | |
| 1142 | | ESJ40086.1 | 26759 | 29988 | 2106 | | | | | | | | | | |
| 1143 | | KNB22031.1 | 26761 | 29990 | 2106 | | | | | | | | | | |
| 1144 | | AJD94187.1 | 26763 | 29992 | 2106 | | | | | | | | | | |
| 1145 | | AJD98581.1 | 26765 | 29994 | 2106 | | | | | | | | | | |
| 1146 | | CEA95105.1 | 26767 | 29996 | 2106 | | | | | | | | | | |
| 1147 | | CEB50843.1 | 26769 | 29998 | 2106 | | | | | | | | | | |
| 1148 | | CDU93374.1 | 26771 | 30000 | 2106 | | | | | | | | | | |
| 1149 | | CEB74773.1 | 26773 | 30002 | 2106 | | | | | | | | | | |
| 1150 | | CED02079.1 | 26775 | 30004 | 2106 | | | | | | | | | | |
| 1151 | | CEC70256.1 | 26777 | 30006 | 2106 | | | | | | | | | | |
| 1152 | | CDZ07208.1 | 26779 | 30008 | 2106 | | | | | | | | | | |
| 1153 | | CED89041.1 | 26781 | 30010 | 2106 | | | | | | | | | | |
| 1154 | | CDV50414.1 | 26783 | 30012 | 2106 | | | | | | | | | | |
| 1155 | | CFW88482.1 | 26785 | 30014 | 2106 | | | | | | | | | | |
| 1156 | | CDZ03619.1 | 26787 | 30016 | 2106 | | | | | | | | | | |
| 1157 | | CEC95426.1 | 26789 | 30018 | 2106 | | | | | | | | | | |
| 1158 | | CDV33411.1 | 26791 | 30020 | 2106 | | | | | | | | | | |
| 1159 | | CEB60520.1 | 26793 | 30022 | 2106 | | | | | | | | | | |
| 1160 | | CEC11581.1 | 26795 | 30024 | 2106 | | | | | | | | | | |
| 1161 | | CEA33004.1 | 26797 | 30026 | 2106 | | | | | | | | | | |
| 1162 | | CDV54740.1 | 26799 | 30028 | 2106 | | | | | | | | | | |
| 1163 | | CEA34479.1 | 26801 | 30030 | 2106 | | | | | | | | | | |
| 1164 | | CED50260.1 | 26803 | 30032 | 2106 | | | | | | | | | | |
| 1165 | | CEC06544.1 | 26805 | 30034 | 2106 | | | | | | | | | | |
| 1166 | | CDY81479.1 | 26807 | 30036 | 2106 | | | | | | | | | | |
| 1167 | | CEC66459.1 | 26809 | 30038 | 2106 | | | | | | | | | | |
| 1168 | | CDU75276.1 | 26811 | 30040 | 2106 | | | | | | | | | | |
| 1169 | | CDV02402.1 | 26813 | 30042 | 2106 | | | | | | | | | | |
| 1170 | | CDU44889.1 | 26815 | 30044 | 2106 | | | | | | | | | | |
| 1171 | | CEA82129.1 | 26817 | 30046 | 2106 | | | | | | | | | | |
| 1172 | | CDV48179.1 | 26819 | 30048 | 2106 | | | | | | | | | | |
| 1173 | | CEA48949.1 | 26821 | 30050 | 2106 | | | | | | | | | | |
| 1174 | | CDU62790.1 | 26823 | 30052 | 2106 | | | | | | | | | | |
| 1175 | | CDV82103.1 | 26825 | 30054 | 2106 | | | | | | | | | | |
| 1176 | | CEC15870.1 | 26827 | 30056 | 2106 | | | | | | | | | | |
| 1177 | | CEB34159.1 | 26829 | 30058 | 2106 | | | | | | | | | | |
| 1178 | | CEC22809.1 | 26831 | 30060 | 2106 | | | | | | | | | | |
| 1179 | | CEC29439.1 | 26833 | 30062 | 2106 | | | | | | | | | | |
| 1180 | | CEC81190.1 | 26835 | 30064 | 2106 | | | | | | | | | | |
| 1181 | | CEC27073.1 | 26837 | 30066 | 2106 | | | | | | | | | | |
| 1182 | | CDV36502.1 | 26839 | 30068 | 2106 | | | | | | | | | | |
| 1183 | | CDV99879.1 | 26841 | 30070 | 2106 | | | | | | | | | | |
| 1184 | | CEB21249.1 | 26843 | 30072 | 2106 | | | | | | | | | | |
| 1185 | | CED29538.1 | 26845 | 30074 | 2106 | | | | | | | | | | |
| 1186 | | CEA65653.1 | 26847 | 30076 | 2106 | | | | | | | | | | |
| 1187 | | CDV77393.1 | 26849 | 30078 | 2106 | | | | | | | | | | |
| 1188 | | CEB99322.1 | 26851 | 30080 | 2106 | | | | | | | | | | |
| 1189 | | CEC87091.1 | 26853 | 30082 | 2106 | | | | | | | | | | |
| 1190 | | CEC03349.1 | 26855 | 30084 | 2106 | | | | | | | | | | |
| 1191 | | CDW68451.1 | 26857 | 30086 | 2106 | | | | | | | | | | |
| 1192 | | CEB36538.1 | 26859 | 30088 | 2106 | | | | | | | | | | |
| 1193 | | CEB92475.1 | 26861 | 30090 | 2106 | | | | | | | | | | |
| 1194 | | CEC70642.1 | 26863 | 30092 | 2106 | | | | | | | | | | |
| 1195 | | CED19417.1 | 26865 | 30094 | 2106 | | | | | | | | | | |
| 1196 | | CDU57393.1 | 26867 | 30096 | 2106 | | | | | | | | | | |
| 1197 | | CEC55614.1 | 26869 | 30098 | 2106 | | | | | | | | | | |
| 1198 | | CDW10769.1 | 26871 | 30100 | 2106 | | | | | | | | | | |
| 1199 | | CED40434.1 | 26873 | 30102 | 2106 | | | | | | | | | | |
| 1200 | | CEA77527.1 | 26875 | 30104 | 2106 | | | | | | | | | | |
| 1201 | | CEC78576.1 | 26877 | 30106 | 2106 | | | | | | | | | | |
| 1202 | | CDV87318.1 | 26879 | 30108 | 2106 | | | | | | | | | | |
| 1203 | | CEA60260.1 | 26881 | 30110 | 2106 | | | | | | | | | | |
| 1204 | | CDU45976.1 | 26883 | 30112 | 2106 | | | | | | | | | | |
| 1205 | | CED26176.1 | 26885 | 30114 | 2106 | | | | | | | | | | |
| 1206 | | CEA55542.1 | 26887 | 30116 | 2106 | | | | | | | | | | |
| 1207 | | CDU57356.1 | 26889 | 30118 | 2106 | | | | | | | | | | |
| 1208 | | CEC22620.1 | 26891 | 30120 | 2106 | | | | | | | | | | |
| 1209 | | CDU81239.1 | 26893 | 30122 | 2106 | | | | | | | | | | |
| 1210 | | CEC22198.1 | 26895 | 30124 | 2106 | | | | | | | | | | |
| 1211 | | CDV00136.1 | 26897 | 30126 | 2106 | | | | | | | | | | |
| 1212 | | CDV32907.1 | 26899 | 30128 | 2106 | | | | | | | | | | |
| 1213 | | CEB32341.1 | 26901 | 30130 | 2106 | | | | | | | | | | |
| 1214 | | CDU66154.1 | 26903 | 30132 | 2106 | | | | | | | | | | |
| 1215 | | CEB23430.1 | 26905 | 30134 | 2106 | | | | | | | | | | |
| 1216 | | CEA69988.1 | 26907 | 30136 | 2106 | | | | | | | | | | |
| 1217 | | CED34529.1 | 26909 | 30138 | 2106 | | | | | | | | | | |
| 1218 | | CDU60154.1 | 26911 | 30140 | 2106 | | | | | | | | | | |
| 1219 | | CEB02150.1 | 26913 | 30142 | 2106 | | | | | | | | | | |
| 1220 | | CDV16494.1 | 26915 | 30144 | 2106 | | | | | | | | | | |
| 1221 | | CEA70647.1 | 26917 | 30146 | 2106 | | | | | | | | | | |
| 1222 | | CEB86467.1 | 26919 | 30148 | 2106 | | | | | | | | | | |
| 1223 | | CDV63738.1 | 26921 | 30150 | 2106 | | | | | | | | | | |
| 1224 | | CEB22316.1 | 26923 | 30152 | 2106 | | | | | | | | | | |
| 1225 | | CDY85018.1 | 26925 | 30154 | 2106 | | | | | | | | | | |
| 1226 | | CDY91991.1 | 26927 | 30156 | 2106 | | | | | | | | | | |
| 1227 | | CDV63669.1 | 26929 | 30158 | 2106 | | | | | | | | | | |
| 1228 | | CDV76959.1 | 26931 | 30160 | 2106 | | | | | | | | | | |
| 1229 | | CDV25167.1 | 26933 | 30162 | 2106 | | | | | | | | | | |
| 1230 | | CEA29072.1 | 26935 | 30164 | 2106 | | | | | | | | | | |
| 1231 | | CEC16650.1 | 26937 | 30166 | 2106 | | | | | | | | | | |
| 1232 | | CDV58579.1 | 26939 | 30168 | 2106 | | | | | | | | | | |
| 1233 | | CDV98717.1 | 26941 | 30170 | 2106 | | | | | | | | | | |
| 1234 | | CED02849.1 | 26943 | 30172 | 2106 | | | | | | | | | | |
| 1235 | | CDU96709.1 | 26945 | 30174 | 2106 | | | | | | | | | | |
| 1236 | | CEB76238.1 | 26947 | 30176 | 2106 | | | | | | | | | | |
| 1237 | | CEC37117.1 | 26949 | 30178 | 2106 | | | | | | | | | | |
| 1238 | | CDU99401.1 | 26951 | 30180 | 2106 | | | | | | | | | | |
| 1239 | | CEB54811.1 | 26953 | 30182 | 2106 | | | | | | | | | | |
| 1240 | | CEC86156.1 | 26955 | 30184 | 2106 | | | | | | | | | | |
| 1241 | | CDV41199.1 | 26957 | 30186 | 2106 | | | | | | | | | | |
| 1242 | | CDV19346.1 | 26959 | 30188 | 2106 | | | | | | | | | | |
| 1243 | | CEA51459.1 | 26961 | 30190 | 2106 | | | | | | | | | | |
| 1244 | | CEA31321.1 | 26963 | 30192 | 2106 | | | | | | | | | | |
| 1245 | | CEC70728.1 | 26965 | 30194 | 2106 | | | | | | | | | | |
| 1246 | | CEB55402.1 | 26967 | 30196 | 2106 | | | | | | | | | | |
| 1247 | | CED14441.1 | 26969 | 30198 | 2106 | | | | | | | | | | |
| 1248 | | CED18823.1 | 26971 | 30200 | 2106 | | | | | | | | | | |
| 1249 | | CEB11068.1 | 26973 | 30202 | 2106 | | | | | | | | | | |
| 1250 | | CEA27404.1 | 26975 | 30204 | 2106 | | | | | | | | | | |
| 1251 | | CED08652.1 | 26977 | 30206 | 2106 | | | | | | | | | | |
| 1252 | | CEB93594.1 | 26979 | 30208 | 2106 | | | | | | | | | | |
| 1253 | | CDY97806.1 | 26981 | 30210 | 2106 | | | | | | | | | | |
| 1254 | | CEA81537.1 | 26983 | 30212 | 2106 | | | | | | | | | | |
| 1255 | | CEA95804.1 | 26985 | 30214 | 2106 | | | | | | | | | | |
| 1256 | | CED39167.1 | 26987 | 30216 | 2106 | | | | | | | | | | |
| 1257 | | CEC46303.1 | 26989 | 30218 | 2106 | | | | | | | | | | |
| 1258 | | CEB93174.1 | 26991 | 30220 | 2106 | | | | | | | | | | |
| 1259 | | CEB06233.1 | 26993 | 30222 | 2106 | | | | | | | | | | |
| 1260 | | CEC59899.1 | 26995 | 30224 | 2106 | | | | | | | | | | |
| 1261 | | CDW11869.1 | 26997 | 30226 | 2106 | | | | | | | | | | |
| 1262 | | CEB87176.1 | 26999 | 30228 | 2106 | | | | | | | | | | |
| 1263 | | CDU53202.1 | 27001 | 30230 | 2106 | | | | | | | | | | |
| 1264 | | CEB57382.1 | 27003 | 30232 | 2106 | | | | | | | | | | |
| 1265 | | CED48957.1 | 27005 | 30234 | 2106 | | | | | | | | | | |
| 1266 | | CDW00036.1 | 27007 | 30236 | 2106 | | | | | | | | | | |
| 1267 | | CDV01782.1 | 27009 | 30238 | 2106 | | | | | | | | | | |
| 1268 | | CEA40515.1 | 27011 | 30240 | 2106 | | | | | | | | | | |
| 1269 | | CEB13671.1 | 27013 | 30242 | 2106 | | | | | | | | | | |
| 1270 | | CEA18907.1 | 27015 | 30244 | 2106 | | | | | | | | | | |
| 1271 | | CDY92810.1 | 27017 | 30246 | 2106 | | | | | | | | | | |
| 1272 | | CEA88151.1 | 27019 | 30248 | 2106 | | | | | | | | | | |
| 1273 | | AKO82197.1 | 27021 | 30250 | 2106 | | | | | | | | | | |
| 1274 | | CAR62300.1 | 27023 | 30252 | 2106 | | | | | | | | | | |
| 1275 | | ESF93194.1 | 27025 | 30254 | 2106 | | | | | | | | | | |
| 1276 | | AAV80042.1 | 27027 | 30256 | 2106 | | | | | | | | | | |
| 1277 | | EPE40530.1 | 27029 | 30258 | 2106 | | | | | | | | | | |
| 1278 | | EPE40312.1 | 27031 | 30260 | 2106 | | | | | | | | | | |
| 1279 | | EPE40094.1 | 27033 | 30262 | 2106 | | | | | | | | | | |
| 1280 | | KSB43365.1 | 27035 | 30264 | 2106 | | | | | | | | | | |
| 1281 | | ELX27348.1 | 27037 | 30266 | 2106 | | | | | | | | | | |
| 1282 | | EHC78169.1 | 27039 | 30268 | 2106 | | | | | | | | | | |
| 1283 | | ESF47592.1 | 27041 | 30270 | 2106 | | | | | | | | | | |
| 1284 | | CUS01661.1 | 27043 | 30272 | 2106 | | | | | | | | | | |
| 1285 | | CUS00363.1 | 27045 | 30274 | 2106 | | | | | | | | | | |
| 1286 | | CUS00365.1 | 27047 | 30276 | 2106 | | | | | | | | | | |
| 1287 | | CUS00364.1 | 27049 | 30278 | 2106 | | | | | | | | | | |
| 1288 | | CBY96201.1 | 27051 | 30280 | 2106 | | | | | | | | | | |
| 1289 | | EDZ29121.1 | 27053 | 30282 | 2106 | | | | | | | | | | |
| 1290 | | KNM15295.1 | 27055 | 30284 | 2106 | | | | | | | | | | |
| 1291 | | KNM69140.1 | 27057 | 30286 | 2106 | | | | | | | | | | |
| 1292 | | KNO58854.1 | 27059 | 30288 | 2106 | | | | | | | | | | |
| 1293 | | KNW78917.1 | 27061 | 30290 | 2106 | | | | | | | | | | |
| 1294 | | KTZ77423.1 | 27063 | 30292 | 2106 | | | | | | | | | | |
| 1295 | | KUC23741.1 | 27065 | 30294 | 2106 | | | | | | | | | | |
| 1296 | | ESF03066.1 | 27067 | 30296 | 2106 | | | | | | | | | | |
| 1297 | | KMJ09049.1 | 27069 | 30298 | 2106 | | | | | | | | | | |
| 1298 | | CVG51413.1 | 27071 | 30300 | 1441 | | | | | | | | | | |
| 1299 | | CVB32145.1 | 27073 | 30302 | 1441 | | | | | | | | | | |
| 1300 | | EZQ69787.1 | 27075 | 30304 | 2106 | | | | | | | | | | |
| 1301 | | EFE97640.1 | 27077 | 30306 | 2084 | | | | | | | | | | |
| 1302 | | KFK98826.1 | 27079 | 30308 | 2114 | | | | | | | | | | |
| 1303 | | KFK92331.1 | 27081 | 30310 | 2114 | | | | | | | | | | |
| 1304 | | ABK48269.1 | 27083 | 30312 | 2120 | | | | | | | | | | |
| 1305 | | AFJ48530.1 | 27085 | 30314 | 2106 | | | | | | | | | | |
| 1306 | | GAB81433.1 | 27087 | 30316 | 2106 | | | | | | | | | | |
| 1307 | | ACP25985.1 | 27089 | 30318 | 2042 | | | | | | | | | | |
| 1308 | | CAN98362.1 | 27091 | 30320 | 2099 | | | | | | | | | | |
| 1309 | | EPR18398.1 | 27093 | 30322 | 1312 | | | | | | | | | | |
| 1310 | | KKW93259.1 | 27095 | 30324 | 1989 | | | | | | | | | | |
| 1311 | | CCW17775.1 | 27097 | 30326 | 1558 | | | | | | | | | | |
| 1312 | | KER36316.1 | 27099 | 30328 | 1987 | | | | | | | | | | |
| 1313 | | KQM97530.1 | 27101 | 30330 | 1999 | | | | | | | | | | |
| 1314 | | KPV39946.1 | 27103 | 30332 | 2084 | | | | | | | | | | |
| 1315 | | KQW61501.1 | 27105 | 30334 | 2113 | | | | | | | | | | |
| 1316 | | KLE23444.1 | 27107 | 30336 | 2066 | | | | | | | | | | |
| 1317 | | EDL70258.1 | 27109 | 30338 | 1452 | | | | | | | | | | |
| 1318 | | KHD25449.1 | 27111 | 30340 | 2145 | | | | | | | | | | |
| 1319 | | KFE26121.1 | 27113 | 30342 | 2068 | | | | | | | | | | |
| 1320 | | KFD94244.1 | 27115 | 30344 | 2068 | | | | | | | | | | |
| 1321 | | EJH60535.1 | 27117 | 30346 | 2104 | | | | | | | | | | |
| 1322 | | EMQ54782.1 | 27119 | 30348 | 2068 | | | | | | | | | | |
| 1323 | | KNA52262.1 | 27121 | 30350 | 2068 | | | | | | | | | | |
| 1324 | | GAD79748.1 | 27123 | 30352 | 2141 | | | | | | | | | | |
| 1325 | | KNY42355.1 | 27125 | 30354 | 2065 | | | | | | | | | | |
| 1326 | | KHF16000.1 | 27127 | 30356 | 2066 | | | | | | | | | | |
| 1327 | | KOF25301.1 | 27129 | 30358 | 2068 | | | | | | | | | | |
| 1328 | | KHT39219.1 | 27131 | 30360 | 2104 | | | | | | | | | | |
| 1329 | | KYN85117.1 | 27133 | 30362 | 2068 | | | | | | | | | | |
| 1330 | | EKM24799.1 | 27135 | 30364 | 2143 | | | | | | | | | | |
| 1331 | | CDU08194.1 | 27137 | 30366 | 2107 | | | | | | | | | | |
| 1332 | | CAV18548.1 | 27139 | 30368 | 2085 | | | | | | | | | | |
| 1333 | | KWU01490.1 | 27141 | 30370 | 2066 | | | | | | | | | | |
| 1334 | | KOO13422.1 | 27143 | 30372 | 2065 | | | | | | | | | | |
| 1335 | | CNI05795.1 | 27145 | 30374 | 2106 | | | | | | | | | | |
| 1336 | | CQJ58418.1 | 27147 | 30376 | 2106 | | | | | | | | | | |
| 1337 | | CQJ21067.1 | 27149 | 30378 | 2109 | | | | | | | | | | |
| 1338 | | CFR01911.1 | 27151 | 30380 | 2106 | | | | | | | | | | |
| 1339 | | CNH31705.1 | 27153 | 30382 | 2106 | | | | | | | | | | |
| 1340 | | CFV22963.1 | 27155 | 30384 | 2106 | | | | | | | | | | |
| 1341 | | KFE39720.1 | 27157 | 30386 | 2106 | | | | | | | | | | |
| 1342 | | AKA39096.1 | 27159 | 30388 | 2106 | | | | | | | | | | |
| 1343 | | EHM51572.1 | 27161 | 30390 | 2082 | | | | | | | | | | |

(Part D)

| | | AO | | | | AS | | | |
|---|---|---|---|---|---|---|---|---|---|
| | | DruG | | | | DruH | | | |
| | AN | protein | AP | AQ | AR | protein | AT | AU | |
| | DNA SEQ | length | DruH | PRT SEQ | DNA SEQ | length | Genomic Start | Genomic End | AV |
| 1 | ID NO: | (aa) | protein_accession | ID NO: | ID NO: | (aa) | Point | Point | Comments |
| 2 | | | | | | | 285161 | 295071 | |
| 3 | | | | | | | 23658 | 35022 | |
| 4 | | | | | | | 122429 | 132246 | |
| 5 | | | | | | | 5336 | 16034 | |
| 6 | | | | | | | 5300 | 15998 | |
| 7 | | | | | | | 4236 | 14105 | |
| 8 | | | | | | | 859665 | 869540 | |
| 9 | | | | | | | 346656 | 356168 | |
| 10 | | | | | | | 43741 | 53633 | |
| 11 | | | | | | | 227293 | 237188 | |
| 12 | | | | | | | 1304116 | 1314874 | |
| 13 | | | | | | | 704089 | 714847 | |
| 14 | | | | | | | 1053641 | 1062444 | |
| 15 | | | | | | | 2593529 | 2604285 | |
| 16 | | | | | | | 102484 | 110617 | |
| 17 | | | | | | | 718104 | 726907 | |
| 18 | | | | | | | 621905 | 629933 | |
| 19 | | | | | | | 58930 | 69532 | |
| 20 | | | | | | | 130247 | 138278 | |
| 21 | | | | | | | 3789 | 14436 | |
| 22 | | | | | | | 7008 | 18501 | |
| 23 | | | | | | | 650 | 9027 | |
| 24 | | | | | | | 5201 | 16574 | |
| 25 | | | | | | | 245534 | 255452 | |
| 26 | | | | | | | 2444 | 10852 | |
| 27 | | | | | | | 252885 | 262420 | |
| 28 | | | | | | | 692119 | 701372 | |
| 29 | | | | | | | 3488434 | 3497693 | |
| 30 | | | | | | | 1381151 | 1390413 | |
| 31 | | | | | | | 26796 | 38135 | |
| 32 | | | | | | | 86252 | 97591 | |
| 33 | | | | | | | 1 | 7760 | |
| 34 | | | | | | | 226285 | 237624 | |
| 35 | | | | | | | 825 | 12164 | |
| 36 | | | | | | | 45166 | 56505 | |
| 37 | | | | | | | 6539 | 17878 | |
| 38 | | | | | | | 2707610 | 2718949 | |
| 39 | | | | | | | 111135 | 122474 | |
| 40 | | | | | | | 26765 | 38104 | |
| 41 | | | | | | | 1618476 | 1629815 | |
| 42 | | | | | | | 389081 | 400420 | |
| 43 | | | | | | | 189561 | 200900 | |
| 44 | | | | | | | 33964 | 45303 | |
| 45 | | | | | | | 316476 | 327815 | |
| 46 | | | | | | | 198709 | 210048 | |
| 47 | | | | | | | 203020 | 214359 | |
| 48 | | | | | | | 3735099 | 3746438 | |
| 49 | | | | | | | 170794 | 182133 | |
| 50 | | | | | | | 170697 | 182036 | |
| 51 | | | | | | | 395842 | 407181 | |
| 52 | | | | | | | 314697 | 326036 | |
| 53 | | | | | | | 2239 | 13578 | |
| 54 | | | | | | | 1513766 | 1525105 | |
| 55 | | | | | | | 318207 | 329546 | |
| 56 | | | | | | | 23620 | 34959 | |
| 57 | | | | | | | 90793 | 102132 | |
| 58 | | | | | | | 1 | 8155 | |
| 59 | | | | | | | 30224 | 41563 | |
| 60 | | | | | | | 31431 | 41533 | |
| 61 | | | | | | | 393726 | 405065 | |
| 62 | | | | | | | 1485066 | 1496405 | |
| 63 | | | | | | | 318709 | 330048 | |
| 64 | | | | | | | 7772 | 17322 | |
| 65 | | | | | | | 57001 | 66608 | |
| 66 | | | | | | | 84320 | 94149 | |
| 67 | | | | | | | 1 | 9036 | |
| 68 | | | | | | | 2431766 | 2441378 | |
| 69 | | | | | | | 298110 | 307722 | |
| 70 | | | | | | | 1 | 9075 | |
| 71 | | | | | | | 68076 | 77685 | |
| 72 | | | | | | | 678847 | 689572 | |
| 73 | | | | | | | 267564 | 278272 | |
| 74 | | | | | | | 993973 | 1004769 | |
| 75 | | | | | | | 1471479 | 1481221 | |
| 76 | | | | | | | 1814944 | 1833410 | |
| 77 | | | | | | | 756187 | 764469 | |
| 78 | | | | | | | 273674 | 283215 | |
| 79 | | | | | | | 33201 | 43136 | |
| 80 | | | | | | | 82360 | 93087 | |
| 81 | | | | | | | 207 | 10677 | |
| 82 | | | | | | | 2573999 | 2584115 | |
| 83 | | | | | | | 17690 | 27795 | |
| 84 | | | | | | | 88811 | 98680 | |
| 85 | | | | | | | 233470 | 243330 | |
| 86 | | | | | | | 1894236 | 1904088 | |
| 87 | | | | | | | 18610 | 29425 | |
| 88 | | | | | | | 225 | 10961 | |
| 89 | | | | | | | 5302 | 15990 | |
| 90 | | | | | | | 48622 | 58258 | |
| 91 | | | | | | | 1226195 | 1236109 | |
| 92 | | | | | | | 340028 | 351370 | |
| 93 | | | | | | | 2729 | 13414 | |
| 94 | | | | | | | 426089 | 435318 | |
| 95 | | | | | | | 245308 | 254531 | |
| 96 | | | | | | | 6222 | 17587 | |
| 97 | | | | | | | 10445 | 17368 | |
| 98 | | | | | | | 1131 | 12496 | |
| 99 | | | | | | | 2602750 | 2614148 | |
| 100 | | | | | | | 27199 | 38358 | |
| 101 | | | | | | | 2402449 | 2413847 | |
| 102 | | | | | | | 383162 | 394525 | |
| 103 | | | | | | | 1671024 | 1682422 | |
| 104 | | | | | | | 137353 | 148718 | |
| 105 | | | | | | | 36724 | 47675 | |
| 106 | | | | | | | 3018853 | 3030235 | |
| 107 | | | | | | | 149958 | 161333 | |
| 108 | | | | | | | 17322 | 28685 | |
| 109 | | | | | | | 150639 | 162037 | |
| 110 | | | | | | | 34865 | 46258 | |
| 111 | | | | | | | 19937 | 31423 | |
| 112 | | | | | | | 2177440 | 2186149 | |
| 113 | | | | | | | 118192 | 128839 | |
| 114 | | | | | | | 443806 | 454453 | |
| 115 | | | | | | | 2764522 | 2775796 | |
| 116 | | | | | | | 8919 | 17199 | |
| 117 | | | | | | | 83723 | 99028 | |
| 118 | | | | | | | 10459 | 21092 | |
| 119 | | | | | | | 5588864 | 5599650 | |
| 120 | | | | | | | 2918005 | 2927268 | |
| 121 | | | | | | | 8795 | 20113 | |
| 122 | | | | | | | 808851 | 820169 | |
| 123 | | | | | | | 232897 | 244248 | |
| 124 | 27659 | 699 | | | | | 112735 | 123898 | |
| 125 | | | | | | | 79463 | 92802 | |
| 126 | | | | | | | 220561 | 233009 | |
| 127 | 27669 | 639 | | | | | 246334 | 258609 | |
| 128 | | | | | | | 1958381 | 1969570 | |
| 129 | | | | | | | 3844801 | 3855950 | |
| 130 | 27679 | 696 | | | | | 7678787 | 7690517 | |
| 131 | 27683 | 689 | | | | | 2014187 | 2025976 | |
| 132 | 27686 | 700 | | | | | 550646 | 561413 | |
| 133 | 27690 | 707 | | | | | 5526 | 17650 | |
| 134 | 27694 | 716 | | | | | 112272 | 128556 | |
| 135 | 27697 | 541 | | | | | 308758 | 321298 | |
| 136 | 27700 | 541 | | | | | 305254 | 317794 | |
| 137 | 27704 | 655 | | | | | 46911 | 59042 | |
| 138 | 27708 | 655 | | | | | 104315 | 116446 | |
| 139 | 27711 | 541 | | | | | 5069 | 17609 | |
| 140 | | | | | | | 27101 | 39571 | |
| 141 | | | | | | | 80817 | 95890 | |
| 142 | | | | | | | 1127089 | 1138713 | |
| 143 | 27724 | 926 | | | | | 4083820 | 4096459 | |
| 144 | 27728 | 696 | | | | | 701693 | 713454 | |
| 145 | 27732 | 698 | | | | | 337549 | 349277 | |
| 146 | | | | | | | 2777 | 14206 | |
| 147 | 27739 | 695 | | | | | 5370773 | 5382607 | |
| 148 | | | | | | | 1480736 | 1493419 | |
| 149 | 27746 | 695 | | | | | 1679797 | 1691556 | |
| 150 | 27750 | 926 | | | | | 220348 | 232987 | |
| 151 | 27754 | 926 | | | | | 6453 | 19092 | |
| 152 | 27758 | 926 | | | | | 46058 | 58697 | |
| 153 | 27761 | 926 | | | | | 46480 | 59113 | |
| 154 | 27765 | 937 | | | | | 96516 | 109246 | |
| 155 | 27769 | 937 | | | | | 1092 | 13822 | |
| 156 | 27773 | 937 | | | | | 96516 | 109246 | |
| 157 | 27777 | 937 | | | | | 96516 | 109246 | |
| 158 | 27781 | 937 | | | | | 96516 | 109246 | |
| 159 | 27785 | 937 | | | | | 96516 | 109246 | |
| 160 | 27789 | 937 | | | | | 96516 | 109246 | |
| 161 | 27793 | 937 | | | | | 96516 | 109246 | |
| 162 | 27797 | 937 | | | | | 96516 | 109246 | |
| 163 | 27801 | 937 | | | | | 96516 | 109246 | |
| 164 | 27805 | 937 | | | | | 96516 | 109246 | |
| 165 | 27809 | 937 | | | | | 96516 | 109246 | |
| 166 | 27813 | 690 | | | | | 581118 | 592872 | |
| 167 | 27817 | 937 | | | | | 115861 | 128591 | |
| 168 | 27821 | 937 | | | | | 96538 | 109268 | |
| 169 | 27825 | 926 | | | | | 1528 | 14167 | |
| 170 | 27829 | 754 | | | | | 4194850 | 4207568 | |
| 171 | | | | | | | 3329 | 19651 | |
| 172 | | | | | | | 39907 | 50236 | |
| 173 | 27839 | 696 | | | | | 2598976 | 2610644 | |
| 174 | 27843 | 937 | | | | | 5340349 | 5353079 | |
| 175 | | | | | | | 236820 | 251252 | |
| 176 | 27850 | 872 | | | | | 66102 | 80532 | |
| 177 | | | | | | | 1203521 | 1214561 | |
| 178 | 27857 | 684 | | | | | 5406366 | 5418388 | |
| 179 | 27861 | 715 | | | | | 4827708 | 4839442 | |
| 180 | | | | | | | 49526 | 60017 | |
| 181 | | | | | | | 3247787 | 3258736 | |
| 182 | | | | | | | 4110265 | 4122706 | |
| 183 | 27874 | 695 | | | | | 163466 | 175215 | |
| 184 | | | | | | | 8136 | 20398 | |
| 185 | | | | | | | 5149282 | 5160143 | |
| 186 | | | | | | | 6943666 | 6954529 | |
| 187 | | | | | | | 115793 | 125399 | |
| 188 | | | | | | | 2162370 | 2172185 | |
| 189 | 27893 | 696 | | | | | 43659 | 55415 | |
| 190 | 27896 | 530 | | | | | 441 | 12638 | |
| 191 | 27900 | 940 | | | | | 1385409 | 1398159 | |
| 192 | 27904 | 931 | | | | | 2442587 | 2455235 | |
| 193 | 27908 | 938 | | | | | 864468 | 877224 | |
| 194 | 27911 | 711 | | | | | 3263838 | 3274393 | |
| 195 | 27914 | 711 | | | | | 4548827 | 4559382 | |
| 196 | 27917 | 711 | | | | | 63393 | 73948 | |
| 197 | 27920 | 711 | | | | | 743 | 11298 | |
| 198 | 27924 | 711 | | | | | 72805 | 84541 | |
| 199 | 27928 | 711 | | | | | 72805 | 84541 | |
| 200 | 27931 | 711 | | | | | 741 | 11296 | |
| 201 | 27935 | 711 | | | | | 72805 | 84541 | |
| 202 | 27939 | 711 | | | | | 72805 | 84541 | |
| 203 | 27943 | 711 | | | | | 70442 | 82178 | |
| 204 | 27947 | 711 | | | | | 37743 | 49479 | |
| 205 | 27951 | 711 | | | | | 982 | 12718 | |
| 206 | 27955 | 711 | | | | | 72805 | 84541 | |
| 207 | 27959 | 711 | | | | | 205420 | 217156 | |
| 208 | 27963 | 711 | | | | | 915 | 12651 | |
| 209 | 27967 | 711 | | | | | 72805 | 84541 | |
| 210 | 27971 | 711 | | | | | 977 | 12713 | |
| 211 | 27975 | 711 | | | | | 72805 | 84541 | |
| 212 | 27978 | 697 | | | | | 5833 | 16378 | |
| 213 | 27982 | 711 | | | | | 915 | 12651 | |
| 214 | 27986 | 711 | | | | | 988 | 12724 | |
| 215 | 27990 | 711 | | | | | 915 | 12651 | |
| 216 | 27994 | 711 | | | | | 72805 | 84541 | |
| 217 | 27997 | 697 | | | | | 5631 | 16176 | |
| 218 | 28001 | 711 | | | | | 72805 | 84541 | |
| 219 | 28005 | 711 | | | | | 72805 | 84541 | |
| 220 | 28009 | 711 | | | | | 915 | 12651 | |
| 221 | 28013 | 711 | | | | | 915 | 12651 | |
| 222 | 28017 | 711 | | | | | 732 | 12468 | |
| 223 | 28021 | 711 | | | | | 72805 | 84541 | |
| 224 | 28024 | 711 | | | | | 2632018 | 2642573 | |
| 225 | 28028 | 711 | | | | | 2471032 | 2482723 | |
| 226 | 28032 | 711 | | | | | 795908 | 807599 | |
| 227 | 28036 | 711 | | | | | 2515204 | 2526895 | |
| 228 | 28040 | 711 | | | | | 3347675 | 3359366 | |
| 229 | 28044 | 711 | | | | | 73643 | 85334 | |
| 230 | 28048 | 697 | | | | | 353197 | 365180 | |
| 231 | 28052 | 711 | | | | | 3317256 | 3328947 | |
| 232 | 28056 | 710 | | | | | 2412440 | 2424132 | |
| 233 | 28060 | 711 | | | | | 328927 | 340618 | |
| 234 | 28063 | 713 | | | | | 90286 | 99824 | |
| 235 | 28066 | 711 | | | | | 3447532 | 3458087 | |
| 236 | 28070 | 711 | | | | | 2935764 | 2947455 | |
| 237 | 28074 | 711 | | | | | 27284 | 38975 | |
| 238 | 28078 | 712 | | | | | 3467956 | 3479700 | |
| 239 | 28082 | 711 | | | | | 128157 | 139861 | |
| 240 | 28086 | 711 | | | | | 39127 | 50831 | |
| 241 | 28090 | 611 | | | | | 18407 | 30437 | |
| 242 | 28094 | 697 | | | | | 18502 | 30466 | |
| 243 | 28098 | 697 | | | | | 119061 | 131025 | |
| 244 | 28102 | 697 | | | | | 18386 | 30416 | |
| 245 | 28105 | 627 | | | | | 1131 | 11369 | |
| 246 | 28109 | 688 | | | | | 51400 | 63317 | |
| 247 | 28112 | 688 | | | | | 25409 | 35277 | |
| 248 | | | | | | | 619907 | 631929 | |
| 249 | 28119 | 926 | | | | | 86254 | 98887 | |
| 250 | 28123 | 698 | | | | | 84624 | 96349 | |
| 251 | 28127 | 698 | | | | | 260934 | 272659 | |
| 252 | 28131 | 698 | | | | | 260934 | 272659 | |
| 253 | 28135 | 926 | | | | | 138803 | 151436 | |
| 254 | 28139 | 698 | | | | | 254096 | 265821 | |
| 255 | 28143 | 698 | | | | | 84624 | 96349 | |
| 256 | 28147 | 698 | | | | | 260934 | 272659 | |
| 257 | 28151 | 698 | | | | | 260934 | 272659 | |
| 258 | 28155 | 698 | | | | | 84624 | 96349 | |
| 259 | 28159 | 698 | | | | | 84624 | 96349 | |
| 260 | 28163 | 698 | | | | | 84624 | 96349 | |
| 261 | 28167 | 698 | | | | | 105497 | 117255 | |
| 262 | 28171 | 698 | | | | | 941313 | 953071 | |
| 263 | 28175 | 926 | | | | | 36048 | 48681 | |
| 264 | 28178 | 926 | | | | | 1 | 10627 | |
| 265 | 28182 | 926 | | | | | 4052 | 16685 | |
| 266 | 28186 | 698 | | | | | 75007 | 86783 | |
| 267 | 28190 | 698 | | | | | 3128238 | 3139966 | |
| 268 | 28194 | 698 | | | | | 117975 | 129703 | |
| 269 | 28198 | 926 | | | | | 30971 | 43604 | |
| 270 | 28202 | 926 | | | | | 55282 | 67915 | |
| 271 | 28205 | 926 | | | | | 1886892 | 1899525 | |
| 272 | 28209 | 926 | | | | | 2821816 | 2834449 | |
| 273 | 28213 | 679 | | | | | 4744370 | 4757010 | |
| 274 | 28217 | 926 | | | | | 4554614 | 4567253 | |
| 275 | 28221 | 926 | | | | | 4646084 | 4658723 | |
| 276 | 28225 | 679 | | | | | 4811280 | 4823920 | |
| 277 | 28229 | 926 | | | | | 149904 | 162543 | |
| 278 | 28233 | 618 | | | | | 11206 | 23846 | |
| 279 | 28237 | 695 | | | | | 4232584 | 4244337 | |
| 280 | 28240 | 695 | | | | | 3948 | 15701 | |
| 281 | 28244 | 693 | | | | | 3412342 | 3424101 | |
| 282 | 28248 | 696 | | | | | 92338 | 104097 | |
| 283 | | | | | | | 3419212 | 3429296 | |
| 284 | | | | | | | 3422352 | 3432436 | |
| 285 | 28258 | 639 | | | | | 16523 | 28443 | |
| 286 | | | | | | | 3523471 | 3533850 | |
| 287 | | | | | | | 1624260 | 1637507 | |
| 288 | | | | | | | 12158 | 24902 | |
| 289 | 28271 | 938 | | | | | 39835 | 52569 | |
| 290 | 28275 | 938 | | | | | 225795 | 238529 | |
| 291 | 28279 | 929 | | | | | 32560 | 45315 | |
| 292 | 28283 | 938 | | | | | 1842 | 14576 | |
| 293 | 28287 | 929 | | | | | 288594 | 301349 | |
| 294 | 28291 | 938 | | | | | 4116123 | 4128871 | |
| 295 | 28295 | 901 | | | | | 101530 | 114278 | |
| 296 | | | KXV70697.1 | 25068 | 28297 | 557 | 3917 | 12134 | |
| 297 | | | AKQ30570.1 | 25070 | 28299 | 1096 | 1915082 | 1924692 | |
| 298 | | | KOP86149.1 | 25072 | 28301 | 1096 | 5407 | 15017 | |
| 299 | | | KRS04353.1 | 25074 | 28303 | 1096 | 11453 | 21063 | |
| 300 | | | KRS02341.1 | 25076 | 28305 | 1096 | 71328 | 80938 | |
| 301 | | | KRS03108.1 | 25078 | 28307 | 1096 | 11453 | 21063 | |
| 302 | | | KRR94713.1 | 25080 | 28309 | 1096 | 29556 | 39166 | |
| 303 | | | KRS09272.1 | 25082 | 28311 | 1096 | 71328 | 80938 | |
| 304 | | | EXE63525.1 | 25084 | 28313 | 1096 | 695254 | 704870 | |
| 305 | | | EXC47767.1 | 25086 | 28315 | 1122 | 5489 | 15158 | |
| 306 | | | ELW93014.1 | 25088 | 28317 | 1096 | 15113 | 24729 | |
| 307 | | | ENW35932.1 | 25090 | 28319 | 404 | 469715 | 477249 | |
| 308 | | | ENW44601.1 | 25092 | 28321 | 1097 | 573781 | 583394 | |
| 309 | | | ENW49013.1 | 25094 | 28323 | 1122 | 463130 | 472796 | |
| 310 | | | EKP68253.1 | 25096 | 28325 | 1122 | 270846 | 280512 | |
| 311 | | | EKL42058.1 | 25098 | 28327 | 1096 | 30124 | 39734 | |
| 312 | | | ENV15229.1 | 25100 | 28329 | 1120 | 3309500 | 3319165 | |
| 313 | | | ENX21438.1 | 25102 | 28331 | 1097 | 509761 | 519374 | |
| 314 | | | ENV06098.1 | 25104 | 28333 | 1122 | 771437 | 781106 | |
| 315 | | | EDS03748.1 | 25106 | 28335 | 1065 | 88763 | 98100 | |
| 316 | | | AMN11449.1 | 25108 | 28337 | 1132 | 1725136 | 1734833 | |
| 317 | | | AFT94988.1 | 25110 | 28339 | 1132 | 1765863 | 1775560 | |
| 318 | | | EGF91080.1 | 25112 | 28341 | 1147 | 331550 | 341110 | |
| 319 | | | ESQ74518.1 | 25114 | 28343 | 1147 | 32073 | 41627 | |
| 320 | | | CAL94924.1 | 25116 | 28345 | 1192 | 2527848 | 2537752 | |
| 321 | | | AJE23875.1 | 25118 | 28347 | 1110 | 197536 | 206001 | |
| 322 | | | CUA18546.1 | 25120 | 28349 | 1065 | 2356943 | 2366283 | |
| 323 | | | CBW22537.1 | 25122 | 28351 | 1065 | 2371910 | 2381250 | |
| 324 | | | EYA28062.1 | 25124 | 28353 | 1065 | 293528 | 302868 | |
| 325 | | | EYA33840.1 | 25126 | 28355 | 1037 | 163999 | 173255 | |
| 326 | | | EXY44319.1 | 25128 | 28357 | 1052 | 187 | 9488 | |
| 327 | | | EYB10317.1 | 25130 | 28359 | 1065 | 1271019 | 1280359 | |
| 328 | | | EXZ67957.1 | 25132 | 28361 | 1037 | 20085 | 29341 | |
| 329 | | | EXY49047.1 | 25134 | 28363 | 1065 | 152 | 9492 | |
| 330 | | | EXY53776.1 | 25136 | 28365 | 1065 | 152 | 9492 | |
| 331 | | | EDS16319.1 | 25138 | 28367 | 1065 | 257362 | 266699 | |
| 332 | | | KXT38968.1 | 25140 | 28369 | 1065 | 23341 | 32678 | |
| 333 | | | EGW44031.1 | 25142 | 28371 | 1115 | 780950 | 790461 | |
| 334 | | | EEA03716.1 | 25144 | 28373 | 1107 | 62115 | 71698 | |
| 335 | | | KVM07645.1 | 25146 | 28375 | 1046 | 33574 | 42969 | |
| 336 | | | KVM11422.1 | 25148 | 28377 | 1046 | 33574 | 42969 | |
| 337 | | | KVM41743.1 | 25150 | 28379 | 1046 | 33574 | 42969 | |
| 338 | | | KVU39939.1 | 25152 | 28381 | 247 | 370491 | 377497 | |
| 339 | | | KVX50940.1 | 25154 | 28383 | 1046 | 44196 | 53591 | |
| 340 | | | CDH43329.1 | 25156 | 28385 | 655 | 4045 | 12202 | |
| 341 | | | KPA18786.1 | 25158 | 28387 | 1131 | 595 | 10180 | |
| 342 | | | AIY12567.1 | 25160 | 28389 | 1006 | 1117511 | 1126448 | |
| 343 | | | EYF08671.1 | 25162 | 28391 | 1135 | 53327 | 62186 | |
| 344 | | | KQM35754.1 | 25164 | 28393 | 995 | 5095 | 13992 | |
| 345 | | | ALD78155.1 | 25166 | 28395 | 212 | 3535505 | 3542469 | |
| 346 | | | KPR54497.1 | 25168 | 28397 | 1099 | 99 | 9732 | |
| 347 | | | KLV49874.1 | 25170 | 28399 | 1204 | 585396 | 595495 | |
| 348 | | | KLV71166.1 | 25172 | 28401 | 1099 | 3121291 | 3130924 | |
| 349 | | | BAO81223.1 | 25174 | 28403 | 1185 | 1467812 | 1477691 | |
| 350 | | | ALB51924.1 | 25176 | 28405 | 581 | 3364334 | 3372410 | |
| 351 | | | AGE87744.1 | 25178 | 28407 | 581 | 3364989 | 3373065 | |
| 352 | | | KJR98716.1 | 25180 | 28409 | 1125 | 158922 | 168526 | |
| 353 | | | KLE94550.1 | 25182 | 28411 | 1099 | 34358 | 43990 | |
| 354 | | | KLF52639.1 | 25184 | 28413 | 1095 | 284140 | 293760 | |
| 355 | | | KLW28644.1 | 25186 | 28415 | 1099 | 100681 | 110313 | |
| 356 | | | KLW04618.1 | 25188 | 28417 | 1099 | 307954 | 317586 | |
| 357 | | | KLQ05989.1 | 25190 | 28419 | 978 | 81311 | 90573 | |
| 358 | | | KJI56237.1 | 25192 | 28421 | 978 | 146027 | 155289 | |
| 359 | | | KLP80554.1 | 25194 | 28423 | 736 | 56455 | 64988 | |
| 360 | | | KLW09756.1 | 25196 | 28425 | 1098 | 216709 | 226328 | |
| 361 | | | KLW05114.1 | 25198 | 28427 | 1098 | 663448 | 673067 | |
| 362 | | | KUQ64465.1 | 25200 | 28429 | 1098 | 1 | 9538 | |
| 363 | | | KUQ79075.1 | 25202 | 28431 | 736 | 1 | 8452 | |
| 364 | | | KPS33775.1 | 25204 | 28433 | 1098 | 80541 | 90160 | |
| 365 | | | ESL79956.1 | 25206 | 28435 | 981 | 2192087 | 2201355 | |
| 366 | | | KLW78404.1 | 25208 | 28437 | 736 | 3877717 | 3886250 | |
| 367 | | | KFC20757.1 | 25210 | 28439 | 1007 | 9124 | 18004 | |
| 368 | | | AKQ40860.1 | 25212 | 28441 | 1098 | 94495 | 103750 | |
| 369 | | | EDL49428.1 | 25214 | 28443 | 1099 | 20106 | 29358 | |
| 370 | | | KDM84273.1 | 25216 | 28445 | 978 | 6814 | 16064 | |
| 371 | | | CDU40544.1 | 25218 | 28447 | 1096 | 393610 | 403257 | |
| 372 | | | KHJ28073.1 | 25220 | 28449 | 1096 | 77599 | 87246 | |
| 373 | | | KHI41603.1 | 25222 | 28451 | 978 | 158488 | 167738 | |
| 374 | | | KHH60356.1 | 25224 | 28453 | 1096 | 25725 | 35372 | |
| 375 | | | KHH50449.1 | 25226 | 28455 | 1096 | 78872 | 88519 | |
| 376 | | | KHI22271.1 | 25228 | 28457 | 978 | 6097 | 15347 | |
| 377 | | | KHH70906.1 | 25230 | 28459 | 1096 | 36388 | 46035 | |
| 378 | | | KIG29579.1 | 25232 | 28461 | 978 | 23204 | 32454 | |
| 379 | | | AJE58951.1 | 25234 | 28463 | 1112 | 4932802 | 4942497 | |
| 380 | | | KIY28286.1 | 25236 | 28465 | 978 | 14480 | 23730 | |
| 381 | | | KIZ10080.1 | 25238 | 28467 | 978 | 13870 | 23120 | |
| 382 | | | KJJ46597.1 | 25240 | 28469 | 978 | 70937 | 80187 | |
| 383 | | | KJW55358.1 | 25242 | 28471 | 978 | 141103 | 150353 | |
| 384 | | | KJW51708.1 | 25244 | 28473 | 978 | 107073 | 116323 | |
| 385 | | | KLG52822.1 | 25246 | 28475 | 978 | 104422 | 113672 | |
| 386 | | | KLX28161.1 | 25248 | 28477 | 1096 | 4718573 | 4728220 | |
| 387 | | | KNF16924.1 | 25250 | 28479 | 978 | 6097 | 15347 | |
| 388 | | | KNF21006.1 | 25252 | 28481 | 978 | 104562 | 113812 | |
| 389 | | | KNF39647.1 | 25254 | 28483 | 978 | 6770 | 16020 | |
| 390 | | | KNG41000.1 | 25256 | 28485 | 978 | 150782 | 160032 | |
| 391 | | | KNZ99176.1 | 25258 | 28487 | 1096 | 44216 | 53863 | |
| 392 | | | KOZ12890.1 | 25260 | 28489 | 978 | 82598 | 91848 | |
| 393 | | | KOZ62952.1 | 25262 | 28491 | 978 | 13754 | 23004 | |
| 394 | | | KOZ92097.1 | 25264 | 28493 | 978 | 37881 | 47131 | |
| 395 | | | KOZ11136.1 | 25266 | 28495 | 978 | 13756 | 23006 | |
| 396 | | | KOZ14878.1 | 25268 | 28497 | 978 | 252679 | 261929 | |
| 397 | | | KOZ33113.1 | 25270 | 28499 | 978 | 79917 | 89167 | |
| 398 | | | KOZ23285.1 | 25272 | 28501 | 978 | 351820 | 361070 | |
| 399 | | | KOZ33453.1 | 25274 | 28503 | 978 | 13754 | 23004 | |
| 400 | | | KOZ38041.1 | 25276 | 28505 | 978 | 13755 | 23005 | |
| 401 | | | KOZ50107.1 | 25278 | 28507 | 978 | 351727 | 360977 | |
| 402 | | | KOZ47197.1 | 25280 | 28509 | 978 | 13752 | 23002 | |
| 403 | | | KOZ49679.1 | 25282 | 28511 | 978 | 351882 | 361132 | |
| 404 | | | KOZ66206.1 | 25284 | 28513 | 978 | 71615 | 80865 | |
| 405 | | | KOZ69711.1 | 25286 | 28515 | 978 | 351658 | 360908 | |
| 406 | | | KOZ74971.1 | 25288 | 28517 | 978 | 21854 | 31104 | |
| 407 | | | KOZ84785.1 | 25290 | 28519 | 978 | 70890 | 80140 | |
| 408 | | | KOZ80260.1 | 25292 | 28521 | 978 | 70889 | 80139 | |
| 409 | | | KOZ97703.1 | 25294 | 28523 | 978 | 140404 | 149654 | |
| 410 | | | CTS44167.1 | 25296 | 28525 | 847 | 218 | 9124 | |
| 411 | | | KPH47387.1 | 25298 | 28527 | 978 | 2323 | 11573 | |
| 412 | | | KPH46214.1 | 25300 | 28529 | 978 | 252468 | 261718 | |
| 413 | | | KPH35166.1 | 25302 | 28531 | 978 | 351816 | 361066 | |
| 414 | | | KPH38566.1 | 25304 | 28533 | 978 | 82601 | 91851 | |
| 415 | | | KPO74476.1 | 25306 | 28535 | 978 | 13791 | 23041 | |
| 416 | | | KPP03701.1 | 25308 | 28537 | 978 | 104692 | 113942 | |
| 417 | | | KPP08800.1 | 25310 | 28539 | 978 | 105621 | 114871 | |
| 418 | | | KPP18159.1 | 25312 | 28541 | 978 | 353473 | 362723 | |
| 419 | | | KPP24073.1 | 25314 | 28543 | 978 | 13790 | 23040 | |
| 420 | | | KPP34586.1 | 25316 | 28545 | 978 | 13790 | 23040 | |
| 421 | | | KPP39156.1 | 25318 | 28547 | 978 | 13790 | 23040 | |
| 422 | | | KPP49344.1 | 25320 | 28549 | 978 | 13791 | 23041 | |
| 423 | | | KPP10373.1 | 25322 | 28551 | 978 | 13791 | 23041 | |
| 424 | | | KPP42095.1 | 25324 | 28553 | 978 | 13789 | 23039 | |
| 425 | | | KPP53665.1 | 25326 | 28555 | 978 | 353543 | 362793 | |
| 426 | | | KUH02057.1 | 25328 | 28557 | 978 | 17180 | 26430 | |
| 427 | | | KUH15975.1 | 25330 | 28559 | 734 | 973 | 9534 | |
| 428 | | | KUR31969.1 | 25332 | 28561 | 1096 | 1385 | 11032 | |
| 429 | | | ALZ66919.1 | 25334 | 28563 | 734 | 2305792 | 2314353 | |
| 430 | | | KUT20785.1 | 25336 | 28565 | 1096 | 48863 | 58510 | |
| 431 | | | KUT66045.1 | 25338 | 28567 | 734 | 37927 | 46488 | |
| 432 | | | KUS25251.1 | 25340 | 28569 | 1096 | 36024 | 45671 | |
| 433 | | | KUS40200.1 | 25342 | 28571 | 978 | 12399 | 21649 | |
| 434 | | | KUT25862.1 | 25344 | 28573 | 978 | 18618 | 27868 | |
| 435 | | | KUT18409.1 | 25346 | 28575 | 303 | 18302 | 25527 | |
| 436 | | | KUU15387.1 | 25348 | 28577 | 1096 | 48864 | 58511 | |
| 437 | | | KUT50335.1 | 25350 | 28579 | 978 | 22067 | 31317 | |
| 438 | | | KUX11802.1 | 25352 | 28581 | 1096 | 21668 | 31315 | |
| 439 | | | KUU59476.1 | 25354 | 28583 | 1096 | 34340 | 43987 | |
| 440 | | | KUU32878.1 | 25356 | 28585 | 978 | 18302 | 27552 | |
| 441 | | | KUU24019.1 | 25358 | 28587 | 978 | 12402 | 21652 | |
| 442 | | | KUV88045.1 | 25360 | 28589 | 978 | 12596 | 21846 | |
| 443 | | | KUW19172.1 | 25362 | 28591 | 978 | 93523 | 102773 | |
| 444 | | | KUW69121.1 | 25364 | 28593 | 978 | 50756 | 60006 | |
| 445 | | | KUW47144.1 | 25366 | 28595 | 978 | 18299 | 27549 | |
| 446 | | | KUT58152.1 | 25368 | 28597 | 1096 | 75379 | 85026 | |
| 447 | | | KUX52806.1 | 25370 | 28599 | 1096 | 12564 | 22211 | |
| 448 | | | KUX36551.1 | 25372 | 28601 | 1096 | 44479 | 54126 | |
| 449 | | | AMG81877.1 | 25374 | 28603 | 978 | 4045570 | 4054820 | |
| 450 | | | KXG88716.1 | 25376 | 28605 | 1112 | 36249 | 45944 | |
| 451 | | | KXK90367.1 | 25378 | 28607 | 978 | 372776 | 382026 | |
| 452 | | | KXP75361.1 | 25380 | 28609 | 978 | 50949 | 60199 | |
| 453 | | | KXP57060.1 | 25382 | 28611 | 1096 | 70703 | 80350 | |
| 454 | | | KXP32161.1 | 25384 | 28613 | 850 | 1 | 8912 | |
| 455 | | | KXP24518.1 | 25386 | 28615 | 1096 | 297690 | 307337 | |
| 456 | | | KXR08921.1 | 25388 | 28617 | 524 | 1 | 7932 | |
| 457 | | | KXR32561.1 | 25390 | 28619 | 78 | 297913 | 304506 | |
| 458 | | | KXR26311.1 | 25392 | 28621 | 78 | 297953 | 304546 | |
| 459 | | | KXR92254.1 | 25394 | 28623 | 524 | 298103 | 306035 | |
| 460 | | | KXR64177.1 | 25396 | 28625 | 1096 | 7532 | 17179 | |
| 461 | | | KXR53486.1 | 25398 | 28627 | 1096 | 372284 | 381931 | |
| 462 | | | EKJ54997.1 | 25400 | 28629 | 978 | 13808 | 23058 | |
| 463 | | | ERC53518.1 | 25402 | 28631 | 978 | 83426 | 92676 | |
| 464 | | | ERE00882.1 | 25404 | 28633 | 978 | 83930 | 93180 | |
| 465 | | | ERE21798.1 | 25406 | 28635 | 978 | 81120 | 90370 | |
| 466 | | | ERB68142.1 | 25408 | 28637 | 978 | 80083 | 89333 | |
| 467 | | | ELV15310.1 | 25410 | 28639 | 978 | 13797 | 23047 | |
| 468 | | | EKK91184.1 | 25412 | 28641 | 978 | 10348 | 19598 | |
| 469 | | | EKK67941.1 | 25414 | 28643 | 978 | 79653 | 88903 | |
| 470 | | | EKK62292.1 | 25416 | 28645 | 982 | 79616 | 88878 | |
| 471 | | | EYE30768.1 | 25418 | 28647 | 1119 | 17116 | 26789 | |
| 472 | | | EYE15510.1 | 25420 | 28649 | 1119 | 12483 | 22156 | |
| 473 | | | EYE15597.1 | 25422 | 28651 | 1119 | 12403 | 22076 | |
| 474 | | | ESA76410.1 | 25424 | 28653 | 1112 | 23478 | 33173 | |
| 475 | | | EZJ86914.1 | 25426 | 28655 | 1119 | 3996 | 13669 | |
| 476 | | | KEO21428.1 | 25428 | 28657 | 1119 | 3949 | 13622 | |
| 477 | | | KDA70971.1 | 25430 | 28659 | 1119 | 12782 | 22455 | |
| 478 | | | KDS94687.1 | 25432 | 28661 | 810 | 1402 | 10191 | |
| 479 | | | KDT14123.1 | 25434 | 28663 | 1096 | 29694 | 39341 | |
| 480 | | | KEN70535.1 | 25436 | 28665 | 1096 | 28505 | 38152 | |
| 481 | | | KDV77073.1 | 25438 | 28667 | 1096 | 29694 | 39341 | |
| 482 | | | KDW26987.1 | 25440 | 28669 | 1096 | 37897 | 47544 | |
| 483 | | | KEN84156.1 | 25442 | 28671 | 77 | 824 | 7414 | |
| 484 | | | KEO02788.1 | 25444 | 28673 | 77 | 667 | 7257 | |
| 485 | | | KDX84121.1 | 25446 | 28675 | 77 | 30771 | 37361 | |
| 486 | | | KEJ20442.1 | 25448 | 28677 | 1119 | 12363 | 22036 | |
| 487 | | | KEJ21098.1 | 25450 | 28679 | 1119 | 12378 | 22051 | |
| 488 | | | EMX91852.1 | 25452 | 28681 | 279 | 75564 | 85936 | |
| 489 | | | EMW69585.1 | 25454 | 28683 | 580 | 227 | 8326 | |
| 490 | | | EMW64103.1 | 25456 | 28685 | 580 | 250 | 8349 | |
| 491 | | | EMW45431.1 | 25458 | 28687 | 580 | 353 | 8452 | |
| 492 | | | EMV86374.1 | 25460 | 28689 | 228 | 811 | 7854 | |
| 493 | | | ENA88226.1 | 25462 | 28691 | 580 | 363 | 8462 | |
| 494 | | | ENB03533.1 | 25464 | 28693 | 228 | 1250 | 8293 | |
| 495 | | | ERD09265.1 | 25466 | 28695 | 978 | 14046 | 23296 | |
| 496 | | | EKK37395.1 | 25468 | 28697 | 978 | 13796 | 23046 | |
| 497 | | | ELW28318.1 | 25470 | 28699 | 978 | 13976 | 23226 | |
| 498 | | | KDT83307.1 | 25472 | 28701 | 1119 | 16955 | 26628 | |
| 499 | | | KEK80771.1 | 25474 | 28703 | 1119 | 51355 | 61028 | |
| 500 | | | KEL07352.1 | 25476 | 28705 | 1119 | 15020 | 24693 | |
| 501 | | | KEK94608.1 | 25478 | 28707 | 1119 | 17085 | 26758 | |
| 502 | | | EKK36705.1 | 25480 | 28709 | 978 | 13809 | 23059 | |
| 503 | | | EKI06061.1 | 25482 | 28711 | 978 | 70939 | 80189 | |
| 504 | | | EIL64996.1 | 25484 | 28713 | 734 | 48987 | 57548 | |
| 505 | | | EKI02733.1 | 25486 | 28715 | 978 | 71504 | 80754 | |
| 506 | | | KEL97938.1 | 25488 | 28717 | 1119 | 5160 | 14833 | |
| 507 | | | KEM08516.1 | 25490 | 28719 | 1119 | 17042 | 26715 | |
| 508 | | | KEM49611.1 | 25492 | 28721 | 978 | 17146 | 26396 | |
| 509 | | | KEN01217.1 | 25494 | 28723 | 648 | 970 | 9273 | |
| 510 | | | KEM09156.1 | 25496 | 28725 | 648 | 1056 | 9359 | |
| 511 | | | KEM25600.1 | 25498 | 28727 | 648 | 1069 | 9372 | |
| 512 | | | KEM85725.1 | 25500 | 28729 | 1119 | 16764 | 26437 | |
| 513 | | | KEN65097.1 | 25502 | 28731 | 1119 | 4411 | 14084 | |
| 514 | | | ELW21806.1 | 25504 | 28733 | 978 | 23163 | 32413 | |
| 515 | | | EKK71768.1 | 25506 | 28735 | 738 | 72931 | 81461 | |
| 516 | | | EKK61090.1 | 25508 | 28737 | 978 | 13790 | 23040 | |
| 517 | | | EKK52334.1 | 25510 | 28739 | 978 | 13792 | 23042 | |
| 518 | | | EKK87668.1 | 25512 | 28741 | 978 | 81736 | 90986 | |
| 519 | | | EKV85100.1 | 25514 | 28743 | 978 | 13804 | 23054 | |
| 520 | | | EKV70654.1 | 25516 | 28745 | 978 | 13802 | 23052 | |
| 521 | | | EKW05773.1 | 25518 | 28747 | 978 | 79877 | 89127 | |
| 522 | | | EKW03271.1 | 25520 | 28749 | 978 | 13798 | 23048 | |
| 523 | | | ESD10540.1 | 25522 | 28751 | 1104 | 4185 | 13813 | |
| 524 | | | ESA83484.1 | 25524 | 28753 | 1112 | 48996 | 58691 | |
| 525 | | | ESD19200.1 | 25526 | 28755 | 399 | 1 | 7559 | |
| 526 | | | ESD45642.1 | 25528 | 28757 | 1112 | 48833 | 58528 | |
| 527 | | | ESD61125.1 | 25530 | 28759 | 589 | 18577 | 26703 | |
| 528 | | | ESD71192.1 | 25532 | 28761 | 1104 | 20331 | 29959 | |
| 529 | | | ESD92013.1 | 25534 | 28763 | 1112 | 60634 | 70329 | |
| 530 | | | ESD92685.1 | 25536 | 28765 | 1112 | 1234 | 10929 | |
| 531 | | | EKW04268.1 | 25538 | 28767 | 978 | 13806 | 23056 | |
| 532 | | | EKW19025.1 | 25540 | 28769 | 978 | 13808 | 23058 | |
| 533 | | | EIN33838.1 | 25542 | 28771 | 982 | 13788 | 23050 | |
| 534 | | | EKW08488.1 | 25544 | 28773 | 978 | 13808 | 23058 | |
| 535 | | | ELW29952.1 | 25546 | 28775 | 978 | 2439 | 11689 | |
| 536 | | | EKW22422.1 | 25548 | 28777 | 233 | 55 | 7070 | |
| 537 | | | EKW74795.1 | 25550 | 28779 | 978 | 13790 | 23040 | |
| 538 | | | EKY34592.1 | 25552 | 28781 | 978 | 13806 | 23056 | |
| 539 | | | EKW38125.1 | 25554 | 28783 | 978 | 13800 | 23050 | |
| 540 | | | EKW51347.1 | 25556 | 28785 | 978 | 13792 | 23042 | |
| 541 | | | EKW70676.1 | 25558 | 28787 | 978 | 79810 | 89060 | |
| 542 | | | EKW68014.1 | 25560 | 28789 | 982 | 14005 | 23267 | |
| 543 | | | EKW71372.1 | 25562 | 28791 | 978 | 10852 | 20102 | |
| 544 | | | EKY34709.1 | 25564 | 28793 | 1117 | 416 | 10083 | |
| 545 | | | EKW75843.1 | 25566 | 28795 | 978 | 13959 | 23209 | |
| 546 | | | ELW46372.1 | 25568 | 28797 | 978 | 13998 | 23248 | |
| 547 | | | EKW96657.1 | 25570 | 28799 | 978 | 79287 | 88537 | |
| 548 | | | EKW96261.1 | 25572 | 28801 | 978 | 13993 | 23243 | |
| 549 | | | ELV31250.1 | 25574 | 28803 | 978 | 30317 | 39567 | |
| 550 | | | ELV35524.1 | 25576 | 28805 | 978 | 30154 | 39404 | |
| 551 | | | ELV44384.1 | 25578 | 28807 | 978 | 842 | 10092 | |
| 552 | | | ELW27708.1 | 25580 | 28809 | 978 | 13946 | 23196 | |
| 553 | | | ELV47168.1 | 25582 | 28811 | 978 | 13933 | 23183 | |
| 554 | | | ELW24263.1 | 25584 | 28813 | 1119 | 4040 | 13713 | |
| 555 | | | ELV62897.1 | 25586 | 28815 | 978 | 19714 | 28964 | |
| 556 | | | ETJ70883.1 | 25588 | 28817 | 1129 | 37993 | 47697 | |
| 557 | | | ELV63260.1 | 25590 | 28819 | 978 | 4590 | 13840 | |
| 558 | | | ACA79489.1 | 25592 | 28821 | 1119 | 4256022 | 4265695 | |
| 559 | | | ETJ79054.1 | 25594 | 28823 | 287 | 15819 | 22996 | |
| 560 | | | ERB88975.1 | 25596 | 28825 | 978 | 13919 | 23169 | |
| 561 | | | ERD08337.1 | 25598 | 28827 | 978 | 14296 | 23546 | |
| 562 | | | ERD05021.1 | 25600 | 28829 | 978 | 14116 | 23366 | |
| 563 | | | ERD20748.1 | 25602 | 28831 | 978 | 14235 | 23485 | |
| 564 | | | ERD22951.1 | 25604 | 28833 | 978 | 14065 | 23315 | |
| 565 | | | ERB79217.1 | 25606 | 28835 | 978 | 81295 | 90545 | |
| 566 | | | ERD23992.1 | 25608 | 28837 | 978 | 13981 | 23231 | |
| 567 | | | ERD35316.1 | 25610 | 28839 | 978 | 14139 | 23389 | |
| 568 | | | ERD37185.1 | 25612 | 28841 | 978 | 14019 | 23269 | |
| 569 | | | ERD26786.1 | 25614 | 28843 | 1119 | 19748 | 29421 | |
| 570 | | | ERD52931.1 | 25616 | 28845 | 978 | 14048 | 23298 | |
| 571 | | | ERD40232.1 | 25618 | 28847 | 978 | 14087 | 23337 | |
| 572 | | | ERD45129.1 | 25620 | 28849 | 978 | 14048 | 23298 | |
| 573 | | | ERB91861.1 | 25622 | 28851 | 978 | 81300 | 90550 | |
| 574 | | | ERC00104.1 | 25624 | 28853 | 978 | 81417 | 90667 | |
| 575 | | | ERC08312.1 | 25626 | 28855 | 978 | 14059 | 23309 | |
| 576 | | | ERC07682.1 | 25628 | 28857 | 978 | 13951 | 23201 | |
| 577 | | | ERC15339.1 | 25630 | 28859 | 978 | 13964 | 23214 | |
| 578 | | | ERC23446.1 | 25632 | 28861 | 359 | 1 | 7395 | |
| 579 | | | ERC10660.1 | 25634 | 28863 | 978 | 13969 | 23219 | |
| 580 | | | ERC32306.1 | 25636 | 28865 | 978 | 13936 | 23186 | |
| 581 | | | ERD72599.1 | 25638 | 28867 | 978 | 14036 | 23286 | |
| 582 | | | ERD68377.1 | 25640 | 28869 | 978 | 13984 | 23234 | |
| 583 | | | ERD73456.1 | 25642 | 28871 | 978 | 14079 | 23329 | |
| 584 | | | ERD83189.1 | 25644 | 28873 | 978 | 13972 | 23222 | |
| 585 | | | ERC25311.1 | 25646 | 28875 | 978 | 253340 | 262590 | |
| 586 | | | ERC30061.1 | 25648 | 28877 | 978 | 253529 | 262779 | |
| 587 | | | ERD87991.1 | 25650 | 28879 | 978 | 14029 | 23279 | |
| 588 | | | ERD91777.1 | 25652 | 28881 | 978 | 14154 | 23404 | |
| 589 | | | ERD98684.1 | 25654 | 28883 | 978 | 14071 | 23321 | |
| 590 | | | ERD88429.1 | 25656 | 28885 | 978 | 14016 | 23266 | |
| 591 | | | ERE36112.1 | 25658 | 28887 | 978 | 14071 | 23321 | |
| 592 | | | ERE23641.1 | 25660 | 28889 | 978 | 4432 | 13682 | |
| 593 | | | ERC33935.1 | 25662 | 28891 | 978 | 256038 | 265288 | |
| 594 | | | ERC40342.1 | 25664 | 28893 | 978 | 252555 | 261805 | |
| 595 | | | ERC48259.1 | 25666 | 28895 | 978 | 317955 | 327205 | |
| 596 | | | ETY20410.1 | 25668 | 28897 | 1096 | 345968 | 355615 | |
| 597 | | | ETY13488.1 | 25670 | 28899 | 1096 | 347007 | 356654 | |
| 598 | | | ETY13649.1 | 25672 | 28901 | 1096 | 75313 | 84960 | |
| 599 | | | ETY08683.1 | 25674 | 28903 | 1096 | 75313 | 84960 | |
| 600 | | | ETX96686.1 | 25676 | 28905 | 1096 | 457262 | 466909 | |
| 601 | | | ERO99483.1 | 25678 | 28907 | 1096 | 5012479 | 5022126 | |
| 602 | | | ETY35401.1 | 25680 | 28909 | 1096 | 347245 | 356892 | |
| 603 | | | ETY29967.1 | 25682 | 28911 | 1096 | 75313 | 84960 | |
| 604 | | | ESL20615.1 | 25684 | 28913 | 1119 | 2595377 | 2605050 | |
| 605 | | | ETX82125.1 | 25686 | 28915 | 1096 | 410253 | 419900 | |
| 606 | | | ETX76975.1 | 25688 | 28917 | 1096 | 3544266 | 3553913 | |
| 607 | | | ETY61261.1 | 25690 | 28919 | 1096 | 75313 | 84960 | |
| 608 | | | EZQ66603.1 | 25692 | 28921 | 1096 | 36360 | 46007 | |
| 609 | | | ETY23956.1 | 25694 | 28923 | 1096 | 70745 | 80392 | |
| 610 | | | EKH96739.1 | 25696 | 28925 | 978 | 13540 | 22790 | |
| 611 | | | CDU33064.1 | 25698 | 28927 | 1096 | 296853 | 306500 | |
| 612 | | | EHV18500.1 | 25700 | 28929 | 1104 | 40989 | 50617 | |
| 613 | | | EIP71502.1 | 25702 | 28931 | 978 | 14462 | 23712 | |
| 614 | | | EKI46647.1 | 25704 | 28933 | 978 | 13801 | 23051 | |
| 615 | | | EKI57123.1 | 25706 | 28935 | 978 | 13803 | 23053 | |
| 616 | | | EKI59125.1 | 25708 | 28937 | 978 | 13801 | 23051 | |
| 617 | | | EIP63440.1 | 25710 | 28939 | 553 | 82334 | 90309 | |
| 618 | | | EIP72314.1 | 25712 | 28941 | 978 | 30328 | 39578 | |
| 619 | | | EKI64840.1 | 25714 | 28943 | 978 | 30338 | 39588 | |
| 620 | | | EKI72764.1 | 25716 | 28945 | 978 | 30328 | 39578 | |
| 621 | | | EKI91871.1 | 25718 | 28947 | 978 | 79661 | 88911 | |
| 622 | | | EKI82676.1 | 25720 | 28949 | 978 | 30380 | 39630 | |
| 623 | | | EKJ05365.1 | 25722 | 28951 | 978 | 30254 | 39504 | |
| 624 | | | EKI92954.1 | 25724 | 28953 | 978 | 116536 | 125786 | |
| 625 | | | EKJ00853.1 | 25726 | 28955 | 978 | 27618 | 36868 | |
| 626 | | | EIP72927.1 | 25728 | 28957 | 978 | 13800 | 23050 | |
| 627 | | | EKJ19826.1 | 25730 | 28959 | 978 | 13802 | 23052 | |
| 628 | | | EKJ20863.1 | 25732 | 28961 | 978 | 13806 | 23056 | |
| 629 | | | EKJ36028.1 | 25734 | 28963 | 978 | 13806 | 23056 | |
| 630 | | | EKJ47664.1 | 25736 | 28965 | 978 | 13808 | 23058 | |
| 631 | | | EKJ36500.1 | 25738 | 28967 | 978 | 13802 | 23052 | |
| 632 | | | EIP38854.1 | 25740 | 28969 | 978 | 79651 | 88901 | |
| 633 | | | EIO92502.1 | 25742 | 28971 | 978 | 30370 | 39620 | |
| 634 | | | EIO87495.1 | 25744 | 28973 | 978 | 30410 | 39660 | |
| 635 | | | EIP27285.1 | 25746 | 28975 | 978 | 858 | 10108 | |
| 636 | | | EIP10340.1 | 25748 | 28977 | 978 | 13800 | 23050 | |
| 637 | | | EIP33943.1 | 25750 | 28979 | 978 | 13798 | 23048 | |
| 638 | | | EIP40273.1 | 25752 | 28981 | 978 | 13804 | 23054 | |
| 639 | | | EIP48989.1 | 25754 | 28983 | 978 | 13794 | 23044 | |
| 640 | | | EIP35348.1 | 25756 | 28985 | 978 | 30343 | 39593 | |
| 641 | | | EIP50031.1 | 25758 | 28987 | 978 | 30347 | 39597 | |
| 642 | | | EKI03042.1 | 25760 | 28989 | 978 | 13596 | 22846 | |
| 643 | | | EST65290.1 | 25762 | 28991 | 734 | 297776 | 306337 | |
| 644 | | | EMX76367.1 | 25764 | 28993 | 191 | 49211 | 56397 | |
| 645 | | | EMX78113.1 | 25766 | 28995 | 443 | 53674 | 61616 | |
| 646 | | | EKH36316.1 | 25768 | 28997 | 978 | 13812 | 23062 | |
| 647 | | | EIN15910.1 | 25770 | 28999 | 978 | 13808 | 23058 | |
| 648 | | | EKH25287.1 | 25772 | 29001 | 978 | 332440 | 341690 | |
| 649 | | | EKH13353.1 | 25774 | 29003 | 978 | 13810 | 23060 | |
| 650 | | | EIN16633.1 | 25776 | 29005 | 978 | 384 | 9634 | |
| 651 | | | EIN33028.1 | 25778 | 29007 | 978 | 392 | 9642 | |
| 652 | | | EKH48831.1 | 25780 | 29009 | 978 | 13808 | 23058 | |
| 653 | | | EKJ48108.1 | 25782 | 29011 | 978 | 13813 | 23063 | |
| 654 | | | EFI22363.1 | 25784 | 29013 | 580 | 393643 | 401742 | |
| 655 | | | EFF02930.1 | 25786 | 29015 | 580 | 394852 | 402951 | |
| 656 | | | EQN36979.1 | 25788 | 29017 | 1096 | 424617 | 434264 | |
| 657 | | | EQQ54037.1 | 25790 | 29019 | 1096 | 103333 | 112980 | |
| 658 | | | EQQ62152.1 | 25792 | 29021 | 1096 | 309247 | 316458 | |
| 659 | | | EQQ82444.1 | 25794 | 29023 | 1096 | 390614 | 400261 | |
| 660 | | | EQR15649.1 | 25796 | 29025 | 1096 | 438811 | 448458 | |
| 661 | | | EQR29833.1 | 25798 | 29027 | 1096 | 391018 | 400665 | |
| 662 | | | EQR56341.1 | 25800 | 29029 | 1096 | 391165 | 400812 | |
| 663 | | | EQR70530.1 | 25802 | 29031 | 1096 | 553783 | 563430 | |
| 664 | | | EQR73387.1 | 25804 | 29033 | 1096 | 4977594 | 4987241 | |
| 665 | | | ESP12227.1 | 25806 | 29035 | 1119 | 351091 | 360764 | |
| 666 | | | EQR98264.1 | 25808 | 29037 | 1096 | 2757793 | 2767440 | |
| 667 | | | EQS19619.1 | 25810 | 29039 | 1096 | 427754 | 437401 | |
| 668 | | | EQS44553.1 | 25812 | 29041 | 1096 | 3133555 | 3143202 | |
| 669 | | | EQS43787.1 | 25814 | 29043 | 1096 | 1053884 | 1063531 | |
| 670 | | | ERA63200.1 | 25816 | 29045 | 1096 | 388536 | 398183 | |
| 671 | | | EQS73533.1 | 25818 | 29047 | 1096 | 430980 | 440627 | |
| 672 | | | EQS83532.1 | 25820 | 29049 | 1096 | 426028 | 435675 | |
| 673 | | | EQT06518.1 | 25822 | 29051 | 1096 | 102775 | 112422 | |
| 674 | | | EQT31740.1 | 25824 | 29053 | 1119 | 352526 | 362199 | |
| 675 | | | EQT33090.1 | 25826 | 29055 | 1096 | 3599701 | 3609348 | |
| 676 | | | EQT84559.1 | 25828 | 29057 | 1119 | 636062 | 645735 | |
| 677 | | | EQU07295.1 | 25830 | 29059 | 1096 | 262560 | 272207 | |
| 678 | | | EQU46107.1 | 25832 | 29061 | 847 | 2189631 | 2198531 | |
| 679 | | | EQU56270.1 | 25834 | 29063 | 1096 | 388758 | 398405 | |
| 680 | | | EQU82065.1 | 25836 | 29065 | 1096 | 4855064 | 4864711 | |
| 681 | | | EQN74805.1 | 25838 | 29067 | 1096 | 658360 | 668007 | |
| 682 | | | EQV15998.1 | 25840 | 29069 | 516 | 393529 | 401436 | |
| 683 | | | EQO01911.1 | 25842 | 29071 | 1096 | 430981 | 440628 | |
| 684 | | | EQO57393.1 | 25844 | 29073 | 1119 | 624054 | 633727 | |
| 685 | | | EQO66404.1 | 25846 | 29075 | 1119 | 1181503 | 1191176 | |
| 686 | | | EQO74549.1 | 25848 | 29077 | 1096 | 3758553 | 3768200 | |
| 687 | | | EQP20053.1 | 25850 | 29079 | 1096 | 388587 | 398234 | |
| 688 | | | EQP30925.1 | 25852 | 29081 | 1096 | 388774 | 398421 | |
| 689 | | | EQP87866.1 | 25854 | 29083 | 1096 | 3466364 | 3476011 | |
| 690 | | | EQV39984.1 | 25856 | 29085 | 1096 | 73576 | 83223 | |
| 691 | | | EQV77953.1 | 25858 | 29087 | 1096 | 2087647 | 2097294 | |
| 692 | | | EQV75317.1 | 25860 | 29089 | 1096 | 4273553 | 4283200 | |
| 693 | | | EQV93984.1 | 25862 | 29091 | 1096 | 3719713 | 3729360 | |
| 694 | | | EOX00082.1 | 25864 | 29093 | 1096 | 390270 | 399917 | |
| 695 | | | EOW11794.1 | 25866 | 29095 | 660 | 1242010 | 1250349 | |
| 696 | | | ELF04267.1 | 25868 | 29097 | 1096 | 558868 | 568515 | |
| 697 | | | ELI36531.1 | 25870 | 29099 | 1096 | 36436 | 46083 | |
| 698 | | | ELI49201.1 | 25872 | 29101 | 1119 | 4408 | 14081 | |
| 699 | | | EOW43091.1 | 25874 | 29103 | 1096 | 709076 | 718723 | |
| 700 | | | ELI49521.1 | 25876 | 29105 | 1096 | 29428 | 39075 | |
| 701 | | | EOW53695.1 | 25878 | 29107 | 362 | 410393 | 417840 | |
| 702 | | | EOW53660.1 | 25880 | 29109 | 362 | 460969 | 468416 | |
| 703 | | | EOW60867.1 | 25882 | 29111 | 1096 | 64416 | 74063 | |
| 704 | | | ELG97962.1 | 25884 | 29113 | 717 | 2669732 | 2678242 | |
| 705 | | | ELH03273.1 | 25886 | 29115 | 1096 | 560405 | 570052 | |
| 706 | | | ELF28433.1 | 25888 | 29117 | 1119 | 2025589 | 2035262 | |
| 707 | | | ELJ30966.1 | 25890 | 29119 | 1096 | 40359 | 50006 | |
| 708 | | | EOW72674.1 | 25892 | 29121 | 894 | 398221 | 407262 | |
| 709 | | | ELC68672.1 | 25894 | 29123 | 1096 | 435063 | 444710 | |
| 710 | | | ELC04769.1 | 25896 | 29125 | 1096 | 390301 | 399948 | |
| 711 | | | EOV27242.1 | 25898 | 29127 | 1096 | 64489 | 74136 | |
| 712 | | | ELH63191.1 | 25900 | 29129 | 1119 | 392427 | 402100 | |
| 713 | | | ELD00701.1 | 25902 | 29131 | 1096 | 695401 | 705048 | |
| 714 | | | ELD16726.1 | 25904 | 29133 | 1096 | 2591525 | 2601172 | |
| 715 | | | EOV39941.1 | 25906 | 29135 | 1096 | 1942871 | 1952518 | |
| 716 | | | EOX13559.1 | 25908 | 29137 | 894 | 1232970 | 1242011 | |
| 717 | | | ELD74208.1 | 25910 | 29139 | 1096 | 3687026 | 3696673 | |
| 718 | | | ELC51201.1 | 25912 | 29141 | 1096 | 61006 | 70653 | |
| 719 | | | ELC65366.1 | 25914 | 29143 | 1096 | 410891 | 420538 | |
| 720 | | | ELG08156.1 | 25916 | 29145 | 1119 | 1535102 | 1544775 | |
| 721 | | | ELE06289.1 | 25918 | 29147 | 221 | 439185 | 445994 | |
| 722 | | | ELE13900.1 | 25920 | 29149 | 1096 | 2561123 | 2570770 | |
| 723 | | | EOV54809.1 | 25922 | 29151 | 1096 | 91839 | 101486 | |
| 724 | | | ELE75377.1 | 25924 | 29153 | 1096 | 655020 | 664667 | |
| 725 | | | EU61314.1 | 25926 | 29155 | 1096 | 36434 | 46081 | |
| 726 | | | ELE76411.1 | 25928 | 29157 | 1096 | 390340 | 399987 | |
| 727 | | | EGI23326.1 | 25930 | 29159 | 1112 | 73149 | 82844 | |
| 728 | | | EIF87147.1 | 25932 | 29161 | 1119 | 451385 | 461058 | |
| 729 | | | KDG67766.1 | 25934 | 29163 | 380 | 3753850 | 3761349 | |
| 730 | | | EFK89792.1 | 25936 | 29165 | 1112 | 30388 | 40083 | |
| 731 | | | EFK27049.1 | 25938 | 29167 | 978 | 7440 | 16690 | |
| 732 | | | EFI89546.1 | 25940 | 29169 | 295 | 155 | 7399 | |
| 733 | | | EFJ72999.1 | 25942 | 29171 | 1112 | 22606 | 32301 | |
| 734 | | | EKH61409.1 | 25944 | 29173 | 978 | 13807 | 23057 | |
| 735 | | | EKJ52944.1 | 25946 | 29175 | 978 | 13796 | 23046 | |
| 736 | | | EMS03601.1 | 25948 | 29177 | 1119 | 11246 | 20919 | |
| 737 | | | KDM81020.1 | 25950 | 29179 | 978 | 156383 | 165633 | |
| 738 | | | AHY73975.1 | 25952 | 29181 | 1119 | 5408661 | 5418334 | |
| 739 | | | AHG12156.1 | 25954 | 29183 | 1119 | 5408663 | 5418336 | |
| 740 | | | EYZ76663.1 | 25956 | 29185 | 978 | 104414 | 113664 | |
| 741 | | | EYW98551.1 | 25958 | 29187 | 978 | 140023 | 149273 | |
| 742 | | | EYV59036.1 | 25960 | 29189 | 978 | 140415 | 149665 | |
| 743 | | | EZE90568.1 | 25962 | 29191 | 978 | 7566 | 16816 | |
| 744 | | | EYV46018.1 | 25964 | 29193 | 978 | 106704 | 115954 | |
| 745 | | | EYV44257.1 | 25966 | 29195 | 978 | 139248 | 148498 | |
| 746 | | | EYV24874.1 | 25968 | 29197 | 978 | 140321 | 149571 | |
| 747 | | | EYV22944.1 | 25970 | 29199 | 978 | 8920 | 18170 | |
| 748 | | | EYV18169.1 | 25972 | 29201 | 978 | 106497 | 115747 | |
| 749 | | | EYV20079.1 | 25974 | 29203 | 978 | 7135 | 16385 | |
| 750 | | | EYV16483.1 | 25976 | 29205 | 978 | 139329 | 148579 | |
| 751 | | | EYZ14530.1 | 25978 | 29207 | 978 | 6648 | 15898 | |
| 752 | | | EZQ47393.1 | 25980 | 29209 | 978 | 302436 | 311686 | |
| 753 | | | EZQ50283.1 | 25982 | 29211 | 978 | 351950 | 361200 | |
| 754 | | | EFX13463.1 | 25984 | 29213 | 978 | 293415 | 302665 | |
| 755 | | | EFX18240.1 | 25986 | 29215 | 978 | 293366 | 302616 | |
| 756 | | | KKF77972.1 | 25988 | 29217 | 978 | 14601 | 23851 | |
| 757 | | | KKF80704.1 | 25990 | 29219 | 978 | 13870 | 23120 | |
| 758 | | | KKY44755.1 | 25992 | 29221 | 978 | 3452 | 12702 | |
| 759 | | | ALH93946.1 | 25994 | 29223 | 978 | 5197926 | 5207176 | |
| 760 | | | KRQ04868.1 | 25996 | 29225 | 978 | 284710 | 293960 | |
| 761 | | | EYZ52259.1 | 25998 | 29227 | 978 | 351745 | 360995 | |
| 762 | | | EYZ38308.1 | 26000 | 29229 | 978 | 351772 | 361022 | |
| 763 | | | EYZ37415.1 | 26002 | 29231 | 978 | 13749 | 22999 | |
| 764 | | | EYZ36218.1 | 26004 | 29233 | 978 | 351747 | 360997 | |
| 765 | | | EYX13627.1 | 26006 | 29235 | 978 | 13750 | 23000 | |
| 766 | | | EYX12328.1 | 26008 | 29237 | 978 | 351786 | 361036 | |
| 767 | | | EYX02203.1 | 26010 | 29239 | 978 | 13747 | 22997 | |
| 768 | | | EZD85329.1 | 26012 | 29241 | 978 | 80155 | 89405 | |
| 769 | | | EZE88805.1 | 26014 | 29243 | 978 | 14613 | 23863 | |
| 770 | | | EYV70455.1 | 26016 | 29245 | 978 | 129736 | 138986 | |
| 771 | | | EZE89448.1 | 26018 | 29247 | 978 | 251411 | 260661 | |
| 772 | | | EYV67647.1 | 26020 | 29249 | 978 | 302523 | 311773 | |
| 773 | | | EYX70059.1 | 26022 | 29251 | 978 | 250164 | 259414 | |
| 774 | | | EYX57444.1 | 26024 | 29253 | 978 | 351787 | 361037 | |
| 775 | | | EYX54549.1 | 26026 | 29255 | 978 | 351787 | 361037 | |
| 776 | | | EYX48135.1 | 26028 | 29257 | 978 | 351787 | 361037 | |
| 777 | | | EYX44882.1 | 26030 | 29259 | 978 | 351787 | 361037 | |
| 778 | | | EYX43992.1 | 26032 | 29261 | 978 | 13749 | 22999 | |
| 779 | | | EYX33005.1 | 26034 | 29263 | 978 | 351787 | 361037 | |
| 780 | | | EYX30697.1 | 26036 | 29265 | 978 | 351787 | 361037 | |
| 781 | | | EYX27760.1 | 26038 | 29267 | 978 | 13749 | 22999 | |
| 782 | | | EYW85333.1 | 26040 | 29269 | 978 | 351787 | 361037 | |
| 783 | | | EYW83366.1 | 26042 | 29271 | 978 | 351787 | 361037 | |
| 784 | | | EYW75520.1 | 26044 | 29273 | 978 | 13748 | 22998 | |
| 785 | | | EYW71150.1 | 26046 | 29275 | 978 | 13748 | 22998 | |
| 786 | | | EYW67180.1 | 26048 | 29277 | 978 | 351652 | 360902 | |
| 787 | | | EYW64049.1 | 26050 | 29279 | 978 | 351651 | 360901 | |
| 788 | | | EYW57177.1 | 26052 | 29281 | 978 | 351652 | 360902 | |
| 789 | | | EYW49141.1 | 26054 | 29283 | 978 | 13748 | 22998 | |
| 790 | | | EYW49454.1 | 26056 | 29285 | 978 | 13748 | 22998 | |
| 791 | | | EYW43049.1 | 26058 | 29287 | 978 | 351652 | 360902 | |
| 792 | | | EYW39161.1 | 26060 | 29289 | 978 | 13748 | 22998 | |
| 793 | | | EYW34610.1 | 26062 | 29291 | 978 | 13749 | 22999 | |
| 794 | | | EYW25813.1 | 26064 | 29293 | 978 | 351733 | 360983 | |
| 795 | | | EYW27181.1 | 26066 | 29295 | 978 | 79670 | 88920 | |
| 796 | | | EYW24510.1 | 26068 | 29297 | 978 | 80111 | 89361 | |
| 797 | | | EYW09906.1 | 26070 | 29299 | 978 | 351749 | 360999 | |
| 798 | | | EYW13501.1 | 26072 | 29301 | 978 | 80139 | 89389 | |
| 799 | | | EZF08561.1 | 26074 | 29303 | 978 | 351749 | 360999 | |
| 800 | | | EYW07432.1 | 26076 | 29305 | 978 | 13749 | 22999 | |
| 801 | | | EZF05482.1 | 26078 | 29307 | 978 | 351681 | 360931 | |
| 802 | | | EDZ88246.1 | 26080 | 29309 | 978 | 3701544 | 3710794 | |
| 803 | | | EDZ83804.1 | 26082 | 29311 | 978 | 2581794 | 2591044 | |
| 804 | | | EDU68764.1 | 26084 | 29313 | 978 | 75340 | 84590 | |
| 805 | | | EDU54287.1 | 26086 | 29315 | 978 | 83808 | 93058 | |
| 806 | | | ACI36184.1 | 26088 | 29317 | 978 | 5437955 | 5447205 | |
| 807 | | | EDU34142.1 | 26090 | 29319 | 978 | 82700 | 91950 | |
| 808 | | | EDZ77846.1 | 26092 | 29321 | 978 | 2713466 | 2722716 | |
| 809 | | | EDU76195.1 | 26094 | 29323 | 978 | 115023 | 124273 | |
| 810 | | | EDU82677.1 | 26096 | 29325 | 978 | 313683 | 322933 | |
| 811 | | | EDU87436.1 | 26098 | 29327 | 978 | 115901 | 125151 | |
| 812 | | | EDU97874.1 | 26100 | 29329 | 978 | 80068 | 89318 | |
| 813 | | | AAG59483.1 | 26102 | 29331 | 1117 | 5393893 | 5403560 | |
| 814 | | | AIG71773.1 | 26104 | 29333 | 978 | 5413191 | 5422441 | |
| 815 | | | EZA89752.1 | 26106 | 29335 | 978 | 351745 | 360995 | |
| 816 | | | EZB05975.1 | 26108 | 29337 | 978 | 351787 | 361037 | |
| 817 | | | EZB04404.1 | 26110 | 29339 | 978 | 351787 | 361037 | |
| 818 | | | EZB11566.1 | 26112 | 29341 | 978 | 351878 | 361128 | |
| 819 | | | EYV89286.1 | 26114 | 29343 | 978 | 351787 | 361037 | |
| 820 | | | EZB20621.1 | 26116 | 29345 | 978 | 351787 | 361037 | |
| 821 | | | EZB18142.1 | 26118 | 29347 | 978 | 79602 | 88852 | |
| 822 | | | EZB30350.1 | 26120 | 29349 | 978 | 351749 | 360999 | |
| 823 | | | EFX08674.1 | 26122 | 29351 | 978 | 287972 | 297222 | |
| 824 | | | EZB32014.1 | 26124 | 29353 | 978 | 13750 | 23000 | |
| 825 | | | EZB41523.1 | 26126 | 29355 | 978 | 13747 | 22997 | |
| 826 | | | EZB46984.1 | 26128 | 29357 | 978 | 13747 | 22997 | |
| 827 | | | EZB48637.1 | 26130 | 29359 | 978 | 14769 | 24019 | |
| 828 | | | EZB48353.1 | 26132 | 29361 | 488 | 1 | 7782 | |
| 829 | | | EZB52649.1 | 26134 | 29363 | 714 | 20977 | 29436 | |
| 830 | | | EZB74689.1 | 26136 | 29365 | 978 | 14053 | 23303 | |
| 831 | | | EZB73673.1 | 26138 | 29367 | 978 | 13126 | 22376 | |
| 832 | | | EZB87910.1 | 26140 | 29369 | 978 | 14311 | 23561 | |
| 833 | | | EZB80136.1 | 26142 | 29371 | 978 | 351521 | 360771 | |
| 834 | | | EZB81694.1 | 26144 | 29373 | 978 | 13944 | 23194 | |
| 835 | | | EZB95443.1 | 26146 | 29375 | 978 | 146895 | 156145 | |
| 836 | | | EZC17286.1 | 26148 | 29377 | 596 | 351887 | 359991 | |
| 837 | | | EZC37646.1 | 26150 | 29379 | 978 | 351954 | 361204 | |
| 838 | | | EZC28523.1 | 26152 | 29381 | 978 | 79400 | 88650 | |
| 839 | | | EZC43982.1 | 26154 | 29383 | 978 | 251669 | 260919 | |
| 840 | | | EZC48749.1 | 26156 | 29385 | 978 | 351852 | 361102 | |
| 841 | | | EZC62956.1 | 26158 | 29387 | 978 | 63484 | 72734 | |
| 842 | | | EZC70421.1 | 26160 | 29389 | 978 | 14637 | 23887 | |
| 843 | | | EZC77661.1 | 26162 | 29391 | 978 | 14037 | 23287 | |
| 844 | | | EZC64796.1 | 26164 | 29393 | 978 | 352100 | 361350 | |
| 845 | | | EZC83413.1 | 26166 | 29395 | 978 | 14468 | 23718 | |
| 846 | | | EZC90332.1 | 26168 | 29397 | 978 | 13966 | 23216 | |
| 847 | | | EZC92770.1 | 26170 | 29399 | 978 | 14435 | 23685 | |
| 848 | | | EZC86952.1 | 26172 | 29401 | 978 | 13396 | 22646 | |
| 849 | | | EZD00268.1 | 26174 | 29403 | 978 | 82072 | 91322 | |
| 850 | | | EYV84724.1 | 26176 | 29405 | 978 | 352019 | 361269 | |
| 851 | | | EZD08969.1 | 26178 | 29407 | 978 | 351981 | 361231 | |
| 852 | | | EZD17520.1 | 26180 | 29409 | 978 | 352026 | 361276 | |
| 853 | | | EZD15758.1 | 26182 | 29411 | 978 | 351390 | 360640 | |
| 854 | | | EZD19489.1 | 26184 | 29413 | 978 | 351929 | 361179 | |
| 855 | | | EZD75268.1 | 26186 | 29415 | 978 | 13749 | 22999 | |
| 856 | | | BAB38682.1 | 26188 | 29417 | 1117 | 5363890 | 5373557 | |
| 857 | | | AIF96933.1 | 26190 | 29419 | 1117 | 5389312 | 5398979 | |
| 858 | | | AJA29435.1 | 26192 | 29421 | 1117 | 5354079 | 5363746 | |
| 859 | | | ACT75063.1 | 26194 | 29423 | 1117 | 5393599 | 5403266 | |
| 860 | | | EEC29561.1 | 26196 | 29425 | 978 | 1424935 | 1434185 | |
| 861 | | | EYZ63881.1 | 26198 | 29427 | 978 | 12253 | 21503 | |
| 862 | | | EFX22733.1 | 26200 | 29429 | 280 | 1 | 7159 | |
| 863 | | | ADD59518.1 | 26202 | 29431 | 1119 | 5202149 | 5211822 | |
| 864 | | | ENC26728.1 | 26204 | 29433 | 1096 | 30243 | 39890 | |
| 865 | | | ENA10895.1 | 26206 | 29435 | 1096 | 68021 | 77668 | |
| 866 | | | EMZ79274.1 | 26208 | 29437 | 630 | 79591 | 87840 | |
| 867 | | | ENG24752.1 | 26210 | 29439 | 580 | 361 | 8460 | |
| 868 | | | ENG35038.1 | 26212 | 29441 | 580 | 308 | 8407 | |
| 869 | | | ENG38630.1 | 26214 | 29443 | 580 | 299 | 8398 | |
| 870 | | | END33528.1 | 26216 | 29445 | 580 | 225 | 8324 | |
| 871 | | | ENE04445.1 | 26218 | 29447 | 580 | 299 | 8398 | |
| 872 | | | ENG47568.1 | 26220 | 29449 | 648 | 74 | 8377 | |
| 873 | | | ENG50953.1 | 26222 | 29451 | 580 | 274 | 8373 | |
| 874 | | | ENG56304.1 | 26224 | 29453 | 580 | 275 | 8374 | |
| 875 | | | ENG57263.1 | 26226 | 29455 | 580 | 248 | 8347 | |
| 876 | | | ENH40673.1 | 26228 | 29457 | 580 | 249 | 8348 | |
| 877 | | | ENG66729.1 | 26230 | 29459 | 580 | 341 | 8440 | |
| 878 | | | ENG74276.1 | 26232 | 29461 | 648 | 133 | 8436 | |
| 879 | | | EST67461.1 | 26234 | 29463 | 978 | 52346 | 61596 | |
| 880 | | | EST67375.1 | 26236 | 29465 | 978 | 26084 | 35334 | |
| 881 | | | EIN65415.1 | 26238 | 29467 | 515 | 15190 | 23051 | |
| 882 | | | ELV75968.1 | 26240 | 29469 | 978 | 13903 | 23153 | |
| 883 | | | ELV90963.1 | 26242 | 29471 | 1119 | 4036 | 13709 | |
| 884 | | | EIN69804.1 | 26244 | 29473 | 978 | 13804 | 23054 | |
| 885 | | | EIN85520.1 | 26246 | 29475 | 978 | 13808 | 23058 | |
| 886 | | | ELV76554.1 | 26248 | 29477 | 978 | 22913 | 32163 | |
| 887 | | | ELV98806.1 | 26250 | 29479 | 1119 | 13633 | 23306 | |
| 888 | | | EIN94345.1 | 26252 | 29481 | 982 | 80889 | 90151 | |
| 889 | | | EKH63850.1 | 26254 | 29483 | 978 | 13810 | 23060 | |
| 890 | | | EIN88191.1 | 26256 | 29485 | 978 | 30257 | 39507 | |
| 891 | | | EIN89776.1 | 26258 | 29487 | 978 | 116447 | 125697 | |
| 892 | | | EIO09773.1 | 26260 | 29489 | 978 | 13804 | 23054 | |
| 893 | | | EIN49113.1 | 26262 | 29491 | 978 | 30335 | 39585 | |
| 894 | | | EIO07042.1 | 26264 | 29493 | 978 | 13806 | 23056 | |
| 895 | | | EIO07418.1 | 26266 | 29495 | 978 | 13802 | 23052 | |
| 896 | | | EIO11194.1 | 26268 | 29497 | 978 | 13808 | 23058 | |
| 897 | | | EKH20918.1 | 26270 | 29499 | 978 | 13807 | 23057 | |
| 898 | | | ELW23233.1 | 26272 | 29501 | 978 | 4450 | 13700 | |
| 899 | | | EKI33365.1 | 26274 | 29503 | 1119 | 328 | 10001 | |
| 900 | | | EIO24462.1 | 26276 | 29505 | 233 | 60 | 7075 | |
| 901 | | | EKH71762.1 | 26278 | 29507 | 978 | 13807 | 23057 | |
| 902 | | | EIO49153.1 | 26280 | 29509 | 978 | 13808 | 23058 | |
| 903 | | | EKH87902.1 | 26282 | 29511 | 978 | 13810 | 23060 | |
| 904 | | | ELV91643.1 | 26284 | 29513 | 978 | 30222 | 39472 | |
| 905 | | | ELV93151.1 | 26286 | 29515 | 978 | 6984 | 16234 | |
| 906 | | | EKH83462.1 | 26288 | 29517 | 978 | 13812 | 23062 | |
| 907 | | | EIN52367.1 | 26290 | 29519 | 978 | 13804 | 23054 | |
| 908 | | | EKH07682.1 | 26292 | 29521 | 978 | 369039 | 378289 | |
| 909 | | | ELW12425.1 | 26294 | 29523 | 978 | 13985 | 23235 | |
| 910 | | | EIN68647.1 | 26296 | 29525 | 978 | 13804 | 23054 | |
| 911 | | | ACB20170.1 | 26298 | 29527 | 1119 | 4912969 | 4922642 | |
| 912 | | | ERE14329.1 | 26300 | 29529 | 978 | 82268 | 91518 | |
| 913 | | | ERC65824.1 | 26302 | 29531 | 978 | 80921 | 90171 | |
| 914 | | | ERC74188.1 | 26304 | 29533 | 287 | 14108 | 21285 | |
| 915 | | | ERC92369.1 | 26306 | 29535 | 978 | 4611 | 13861 | |
| 916 | | | EGI28994.1 | 26308 | 29537 | 1119 | 296390 | 306063 | |
| 917 | | | EKH95567.1 | 26310 | 29539 | 978 | 13812 | 23062 | |
| 918 | | | EIO46356.1 | 26312 | 29541 | 978 | 245564 | 254814 | |
| 919 | | | EIO79510.1 | 26314 | 29543 | 978 | 7053 | 16303 | |
| 920 | | | EIO65811.1 | 26316 | 29545 | 978 | 13799 | 23055 | |
| 921 | | | EIO79102.1 | 26318 | 29547 | 982 | 81173 | 90435 | |
| 922 | | | EIO63487.1 | 26320 | 29549 | 978 | 254421 | 263671 | |
| 923 | | | EIO69659.1 | 26322 | 29551 | 978 | 14347 | 23597 | |
| 924 | | | EIP05663.1 | 26324 | 29553 | 978 | 11135 | 20385 | |
| 925 | | | EIP03387.1 | 26326 | 29555 | 375 | 1 | 7442 | |
| 926 | | | ERE42062.1 | 26328 | 29557 | 978 | 80488 | 89738 | |
| 927 | | | ERE45410.1 | 26330 | 29559 | 978 | 80936 | 90186 | |
| 928 | | | KDG81461.1 | 26332 | 29561 | 1096 | 4589703 | 4599350 | |
| 929 | | | KDG84324.1 | 26334 | 29563 | 1096 | 577940 | 587587 | |
| 930 | | | ERB08805.1 | 26336 | 29565 | 1119 | 3410757 | 3420430 | |
| 931 | | | ERB13351.1 | 26338 | 29567 | 1119 | 312752 | 322425 | |
| 932 | | | EQX93206.1 | 26340 | 29569 | 1096 | 1299123 | 1308770 | |
| 933 | | | EQY50265.1 | 26342 | 29571 | 333 | 972500 | 979858 | |
| 934 | | | ERB23700.1 | 26344 | 29573 | 580 | 575397 | 583496 | |
| 935 | | | EQY93866.1 | 26346 | 29575 | 580 | 381918 | 390017 | |
| 936 | | | EQZ08531.1 | 26348 | 29577 | 1096 | 4369199 | 4378846 | |
| 937 | | | EQZ94052.1 | 26350 | 29579 | 1096 | 413548 | 423195 | |
| 938 | | | ERA40554.1 | 26352 | 29581 | 1096 | 440488 | 450135 | |
| 939 | | | CAR16063.1 | 26354 | 29583 | 1112 | 5111725 | 5121420 | |
| 940 | | | AFJ31994.1 | 26356 | 29585 | 1117 | 5251667 | 5261334 | |
| 941 | | | KWW01507.1 | 26358 | 29587 | 1096 | 161157 | 170801 | |
| 942 | | | CAQ90579.1 | 26360 | 29589 | 1112 | 3162768 | 3172460 | |
| 943 | | | EGC09237.1 | 26362 | 29591 | 1096 | 152611 | 162258 | |
| 944 | | | EOW71261.1 | 26364 | 29593 | 894 | 3729077 | 3738118 | |
| 945 | | | AJY69763.1 | 26366 | 29595 | 1141 | 2031423 | 2041104 | |
| 946 | | | EDL62311.1 | 26368 | 29597 | 1176 | 180740 | 190521 | |
| 947 | | | EOD77323.1 | 26370 | 29599 | 1172 | 615 | 10570 | |
| 948 | | | KJS14045.1 | 26372 | 29601 | 1149 | 1372 | 10961 | |
| 949 | | | EAQ32829.1 | 26374 | 29603 | 1113 | 307409 | 317059 | |
| 950 | | | KXS34468.1 | 26376 | 29605 | 1113 | 44606 | 54256 | |
| 951 | | | CDG80808.1 | 26378 | 29607 | 1090 | 170928 | 180527 | |
| 952 | | | KMK41964.1 | 26380 | 29609 | 1095 | 35196 | 44816 | |
| 953 | | | KMV95473.1 | 26382 | 29611 | 1117 | 1146551 | 1156230 | |
| 954 | | | EYT05882.1 | 26384 | 29613 | 1117 | 319260 | 328939 | |
| 955 | | | KGK50932.1 | 26386 | 29615 | 1095 | 410829 | 420449 | |
| 956 | | | KHE28070.1 | 26388 | 29617 | 1099 | 5171646 | 5181278 | |
| 957 | | | KHF57367.1 | 26390 | 29619 | 1095 | 316218 | 325838 | |
| 958 | | | KHQ22240.1 | 26392 | 29621 | 1095 | 2386538 | 2396158 | |
| 959 | | | KLZ78428.1 | 26394 | 29623 | 1095 | 5093831 | 5103451 | |
| 960 | | | KMB09548.1 | 26396 | 29625 | 1095 | 318671 | 328291 | |
| 961 | | | KMG65748.1 | 26398 | 29627 | 1117 | 5116317 | 5125996 | |
| 962 | | | KMG69377.1 | 26400 | 29629 | 1095 | 565524 | 575144 | |
| 963 | | | KMH23799.1 | 26402 | 29631 | 1095 | 5137914 | 5147534 | |
| 964 | | | KMH84529.1 | 26404 | 29633 | 1095 | 1291264 | 1300884 | |
| 965 | | | KMX40525.1 | 26406 | 29635 | 1095 | 315214 | 324834 | |
| 966 | | | ALH86087.1 | 26408 | 29637 | 1095 | 3020287 | 3029907 | |
| 967 | | | KRR31028.1 | 26410 | 29639 | 978 | 49625 | 58887 | |
| 968 | | | ALQ84489.1 | 26412 | 29641 | 1095 | 2158397 | 2168017 | |
| 969 | | | ALQ89984.1 | 26414 | 29643 | 1095 | 2150002 | 2159622 | |
| 970 | | | KSX16170.1 | 26416 | 29645 | 1095 | 8524 | 18144 | |
| 971 | | | KSX18602.1 | 26418 | 29647 | 1095 | 8564 | 18184 | |
| 972 | | | KSX52403.1 | 26420 | 29649 | 1095 | 137974 | 147594 | |
| 973 | | | ALP76745.1 | 26422 | 29651 | 1095 | 840774 | 850394 | |
| 974 | | | KSZ32506.1 | 26424 | 29653 | 1095 | 26187 | 35807 | |
| 975 | | | KUF65063.1 | 26426 | 29655 | 1008 | 109 | 9468 | |
| 976 | | | KUG47426.1 | 26428 | 29657 | 1095 | 20414 | 30034 | |
| 977 | | | KXO23871.1 | 26430 | 29659 | 1095 | 14263 | 23883 | |
| 978 | | | KYM26277.1 | 26432 | 29661 | 1095 | 41531 | 51151 | |
| 979 | | | ERN57702.1 | 26434 | 29663 | 983 | 10160 | 19444 | |
| 980 | | | EWE85119.1 | 26436 | 29665 | 1095 | 328445 | 338065 | |
| 981 | | | ESL33174.1 | 26438 | 29667 | 1095 | 5034330 | 5043950 | |
| 982 | | | KDK59887.1 | 26440 | 29669 | 1095 | 315804 | 325424 | |
| 983 | | | CDK69437.1 | 26442 | 29671 | 1096 | 45317 | 54964 | |
| 984 | | | EMH91691.1 | 26444 | 29673 | 1095 | 9198 | 18818 | |
| 985 | | | EOZ63296.1 | 26446 | 29675 | 1095 | 5980 | 15600 | |
| 986 | | | EOY67007.1 | 26448 | 29677 | 1095 | 5980 | 15600 | |
| 987 | | | ESN46752.1 | 26450 | 29679 | 1117 | 360085 | 369764 | |
| 988 | | | EWF66267.1 | 26452 | 29681 | 1095 | 4867354 | 4876974 | |
| 989 | | | ESM58758.1 | 26454 | 29683 | 286 | 1837389 | 1844582 | |
| 990 | | | KDL89511.1 | 26456 | 29685 | 1117 | 324473 | 334152 | |
| 991 | | | AIX83031.1 | 26458 | 29687 | 1095 | 1046321 | 1055941 | |
| 992 | | | AIG86150.1 | 26460 | 29689 | 983 | 2966500 | 2975784 | |
| 993 | | | EKB81086.1 | 26462 | 29691 | 1095 | 363340 | 372960 | |
| 994 | | | ESM10102.1 | 26464 | 29693 | 1095 | 362779 | 372399 | |
| 995 | | | EPO98435.1 | 26466 | 29695 | 1095 | 8713 | 18333 | |
| 996 | | | EPA89974.1 | 26468 | 29697 | 1095 | 25993 | 35613 | |
| 997 | | | EJI89218.1 | 26470 | 29699 | 997 | 541259 | 550532 | |
| 998 | | | KDE37041.1 | 26472 | 29701 | 1095 | 322666 | 332267 | |
| 999 | | | CAH16733.1 | 26474 | 29703 | 1130 | 2836365 | 2846089 | |
| 1000 | | | KTD65147.1 | 26476 | 29705 | 1129 | 286299 | 296026 | |
| 1001 | | | ETX11656.1 | 26478 | 29707 | 1143 | 71478 | 81199 | |
| 1002 | | | ESY80721.1 | 26480 | 29709 | 1033 | 45837 | 54964 | |
| 1003 | | | ESY66318.1 | 26482 | 29711 | 1047 | 196269 | 205519 | |
| 1004 | | | ESY44730.1 | 26484 | 29713 | 1088 | 453521 | 462897 | |
| 1005 | | | KQU80075.1 | 26486 | 29715 | 1774 | 218186 | 229640 | |
| 1006 | | | EMS40058.1 | 26488 | 29717 | 1141 | 3688 | 13384 | |
| 1007 | | | EMS40204.1 | 26490 | 29719 | 1141 | 172603 | 182299 | |
| 1008 | | | KQQ15549.1 | 26492 | 29721 | 1141 | 42064 | 51757 | |
| 1009 | | | KQO59080.1 | 26494 | 29723 | 1161 | 65935 | 75509 | |
| 1010 | | | KQO90880.1 | 26496 | 29725 | 1161 | 25635 | 35221 | |
| 1011 | | | EFV81024.1 | 26498 | 29727 | 1115 | 1071595 | 1080026 | |
| 1012 | | | EHJ60590.1 | 26500 | 29729 | 1099 | 90561 | 99810 | |
| 1013 | | | AIT79633.1 | 26502 | 29731 | 1099 | 1555853 | 1565102 | |
| 1014 | | | CCA92391.1 | 26504 | 29733 | 1099 | 1584078 | 1593321 | |
| 1015 | | | KTS15499.1 | 26506 | 29735 | 978 | 42898 | 52160 | |
| 1016 | | | KTS86095.1 | 26508 | 29737 | 978 | 116960 | 126222 | |
| 1017 | | | EFM21231.1 | 26510 | 29739 | 1117 | 215561 | 225243 | |
| 1018 | | | KGA41247.1 | 26512 | 29741 | 1098 | 133311 | 142924 | |
| 1019 | | | ACX87466.1 | 26514 | 29743 | 1097 | 1819036 | 1828646 | |
| 1020 | | | EAU47587.1 | 26516 | 29745 | 964 | 2465108 | 2474018 | |
| 1021 | | | EAS45762.1 | 26518 | 29747 | 1124 | 716356 | 725945 | |
| 1022 | | | KJM66694.1 | 26520 | 29749 | 1104 | 103727 | 113355 | |
| 1023 | | | KMK11834.1 | 26522 | 29751 | 1104 | 56209 | 65837 | |
| 1024 | | | KMK24778.1 | 26524 | 29753 | 1104 | 35136 | 44764 | |
| 1025 | | | KXU56725.1 | 26526 | 29755 | 1044 | 4561 | 13774 | |
| 1026 | | | EGW47199.1 | 26528 | 29757 | 292 | 245464 | 254742 | |
| 1027 | | | KSX98965.1 | 26530 | 29759 | 1012 | 140775 | 150099 | |
| 1028 | | | KTF11892.1 | 26532 | 29761 | 1090 | 229654 | 239236 | |
| 1029 | | | KGB84629.1 | 26534 | 29763 | 1099 | 215 | 9832 | |
| 1030 | | | KHE32030.1 | 26536 | 29765 | 1019 | 23646 | 32926 | |
| 1031 | | | KQJ75583.1 | 26538 | 29767 | 1111 | 16183 | 25846 | |
| 1032 | | | KRV15978.1 | 26540 | 29769 | 1036 | 3295 | 12626 | |
| 1033 | | | KSD09759.1 | 26542 | 29771 | 1109 | 5385 | 15042 | |
| 1034 | | | KSD19547.1 | 26544 | 29773 | 1036 | 60524 | 69855 | |
| 1035 | | | KSG03395.1 | 26546 | 29775 | 1036 | 438855 | 448186 | |
| 1036 | | | KSG67482.1 | 26548 | 29777 | 1111 | 15322 | 24985 | |
| 1037 | | | KSE90722.1 | 26550 | 29779 | 1036 | 21419 | 30732 | |
| 1038 | | | KSF67272.1 | 26552 | 29781 | 1111 | 15322 | 24985 | |
| 1039 | | | KSG19247.1 | 26554 | 29783 | 1036 | 1688 | 11019 | |
| 1040 | | | KSG75426.1 | 26556 | 29785 | 1036 | 22047 | 31378 | |
| 1041 | | | KSG85748.1 | 26558 | 29787 | 1036 | 21450 | 30781 | |
| 1042 | | | KSH39447.1 | 26560 | 29789 | 1036 | 194095 | 203426 | |
| 1043 | | | KSH60514.1 | 26562 | 29791 | 1036 | 22047 | 31378 | |
| 1044 | | | KSI22085.1 | 26564 | 29793 | 1036 | 22046 | 31377 | |
| 1045 | | | KSI51646.1 | 26566 | 29795 | 1109 | 518066 | 527723 | |
| 1046 | | | KSI82064.1 | 26568 | 29797 | 1111 | 199155 | 208818 | |
| 1047 | | | KSI99556.1 | 26570 | 29799 | 1036 | 449321 | 458652 | |
| 1048 | | | KSJ34445.1 | 26572 | 29801 | 1111 | 21403 | 31066 | |
| 1049 | | | KSK63576.1 | 26574 | 29803 | 1109 | 573017 | 582674 | |
| 1050 | | | KSL54235.1 | 26576 | 29805 | 1036 | 24722 | 34053 | |
| 1051 | | | KSM73158.1 | 26578 | 29807 | 1111 | 23541 | 33204 | |
| 1052 | | | KSM79531.1 | 26580 | 29809 | 1036 | 22039 | 31370 | |
| 1053 | | | KSN72845.1 | 26582 | 29811 | 1036 | 24722 | 34053 | |
| 1054 | | | KSO70094.1 | 26584 | 29813 | 1036 | 8292 | 17605 | |
| 1055 | | | KSQ54942.1 | 26586 | 29815 | 1036 | 31335 | 40666 | |
| 1056 | | | KSQ13844.1 | 26588 | 29817 | 1036 | 443745 | 453076 | |
| 1057 | | | KSK33231.1 | 26590 | 29819 | 1036 | 61118 | 70449 | |
| 1058 | | | KSL81229.1 | 26592 | 29821 | 1036 | 194095 | 203426 | |
| 1059 | | | KSL64952.1 | 26594 | 29823 | 1036 | 22039 | 31370 | |
| 1060 | | | KSM29109.1 | 26596 | 29825 | 1111 | 42134 | 51797 | |
| 1061 | | | KSN07703.1 | 26598 | 29827 | 1111 | 378604 | 388267 | |
| 1062 | | | KSN22463.1 | 26600 | 29829 | 1111 | 16138 | 25801 | |
| 1063 | | | KSN45780.1 | 26602 | 29831 | 1036 | 438815 | 448146 | |
| 1064 | | | KSO16560.1 | 26604 | 29833 | 1111 | 15322 | 24985 | |
| 1065 | | | KSO84936.1 | 26606 | 29835 | 1036 | 605 | 9936 | |
| 1066 | | | KSO99159.1 | 26608 | 29837 | 1111 | 23415 | 33078 | |
| 1067 | | | KSQ29951.1 | 26610 | 29839 | 1036 | 438855 | 448186 | |
| 1068 | | | KSR12022.1 | 26612 | 29841 | 1036 | 3385 | 12716 | |
| 1069 | | | KSR96744.1 | 26614 | 29843 | 1036 | 518449 | 527756 | |
| 1070 | | | KSS25556.1 | 26616 | 29845 | 1028 | 203854 | 213146 | |
| 1071 | | | KSS39722.1 | 26618 | 29847 | 1099 | 12270 | 21887 | |
| 1072 | | | KSR72710.1 | 26620 | 29849 | 1036 | 194095 | 203426 | |
| 1073 | | | KSS45915.1 | 26622 | 29851 | 1109 | 472018 | 481675 | |
| 1074 | | | ALY37666.1 | 26624 | 29853 | 1111 | 4647649 | 4657312 | |
| 1075 | | | ALY62762.1 | 26626 | 29855 | 1111 | 5985465 | 5995128 | |
| 1076 | | | KXC98082.1 | 26628 | 29857 | 1036 | 73491 | 82822 | |
| 1077 | | | KXC86337.1 | 26630 | 29859 | 1036 | 478140 | 487471 | |
| 1078 | | | KXD25984.1 | 26632 | 29861 | 1036 | 24577 | 33908 | |
| 1079 | | | KXD09025.1 | 26634 | 29863 | 1036 | 84962 | 94293 | |
| 1080 | | | KXD14448.1 | 26636 | 29865 | 1036 | 47987 | 57318 | |
| 1081 | | | KXD23305.1 | 26638 | 29867 | 1036 | 24766 | 34097 | |
| 1082 | | | KXC87753.1 | 26640 | 29869 | 1036 | 84679 | 94010 | |
| 1083 | | | KXF38232.1 | 26642 | 29871 | 1036 | 73251 | 82582 | |
| 1084 | | | KXF47447.1 | 26644 | 29873 | 1036 | 84960 | 94291 | |
| 1085 | | | KXF43394.1 | 26646 | 29875 | 1036 | 84964 | 94295 | |
| 1086 | | | KYO95527.1 | 26648 | 29877 | 1111 | 13894 | 23557 | |
| 1087 | | | KAJ18078.1 | 26650 | 29879 | 970 | 68033 | 77166 | |
| 1088 | | | ERY57592.1 | 26652 | 29881 | 1115 | 1472487 | 1482055 | |
| 1089 | | | ERY65606.1 | 26654 | 29883 | 1115 | 3001215 | 3010783 | |
| 1090 | | | ERV59834.1 | 26656 | 29885 | 1115 | 3014713 | 3024281 | |
| 1091 | | | ERY33375.1 | 26658 | 29887 | 1115 | 276729 | 286297 | |
| 1092 | | | ERY37452.1 | 26660 | 29889 | 1115 | 303965 | 313533 | |
| 1093 | | | ERY30830.1 | 26662 | 29891 | 1115 | 323839 | 333407 | |
| 1094 | | | ERW68207.1 | 26664 | 29893 | 1115 | 321053 | 330621 | |
| 1095 | | | ERW13881.1 | 26666 | 29895 | 1115 | 438293 | 447861 | |
| 1096 | | | ERV79733.1 | 26668 | 29897 | 1115 | 316993 | 326561 | |
| 1097 | | | ETV52348.1 | 26670 | 29899 | 1115 | 387845 | 395610 | |
| 1098 | | | ERU88469.1 | 26672 | 29901 | 1115 | 308446 | 318014 | |
| 1099 | | | ERU86604.1 | 26674 | 29903 | 1115 | 308446 | 318014 | |
| 1100 | | | KEA15334.1 | 26676 | 29905 | 1140 | 772974 | 782617 | |
| 1101 | | | ERZ32987.1 | 26678 | 29907 | 1115 | 332366 | 341934 | |
| 1102 | | | GAA19669.1 | 26680 | 29909 | 970 | 5052470 | 5061603 | |
| 1103 | | | EME92222.1 | 26682 | 29911 | 1111 | 15085 | 24748 | |
| 1104 | | | EYU00120.1 | 26684 | 29913 | 1111 | 198162 | 207825 | |
| 1105 | | | EOT13872.1 | 26686 | 29915 | 1028 | 4956028 | 4965320 | |
| 1106 | | | ETU84933.1 | 26688 | 29917 | 1115 | 527654 | 537213 | |
| 1107 | | | ERX16724.1 | 26690 | 29919 | 1115 | 629489 | 639057 | |
| 1108 | | | EWH25348.1 | 26692 | 29921 | 1140 | 1491323 | 1500966 | |
| 1109 | | | GAD64770.1 | 26694 | 29923 | 1115 | 16216 | 25769 | |
| 1110 | | | GAD61322.1 | 26696 | 29925 | 1116 | 202461 | 212017 | |
| 1111 | | | EZQ18205.1 | 26698 | 29927 | 1099 | 419637 | 429263 | |
| 1112 | | | AMO73921.1 | 26700 | 29929 | 1111 | 445996 | 455662 | |
| 1113 | | | KPW38787.1 | 26702 | 29931 | 1158 | 36556 | 46279 | |
| 1114 | | | KQB53692.1 | 26704 | 29933 | 1111 | 1406 | 11069 | |
| 1115 | | | EZI30290.1 | 26706 | 29935 | 1099 | 22889 | 32461 | |
| 1116 | | | KIR22317.1 | 26708 | 29937 | 1099 | 33962 | 43528 | |
| 1117 | | | KJH86852.1 | 26710 | 29939 | 1029 | 114387 | 123676 | |
| 1118 | | | KWV84208.1 | 26712 | 29941 | 1029 | 9497 | 18786 | |
| 1119 | | | EFQ63351.1 | 26714 | 29943 | 634 | 3228353 | 3236566 | |
| 1120 | | | KMM97952.1 | 26716 | 29945 | 1111 | 145121 | 154784 | |
| 1121 | | | ENY78085.1 | 26718 | 29947 | 1043 | 45721 | 55116 | |
| 1122 | | | ENA27916.1 | 26720 | 29949 | 1116 | 122129 | 131384 | |
| 1123 | | | EWC41654.1 | 26722 | 29951 | 1140 | 1199767 | 1208507 | |
| 1124 | | | EMD98965.1 | 26724 | 29953 | 1027 | 61976 | 71271 | |
| 1125 | | | KPB88881.1 | 26726 | 29955 | 794 | 33409 | 40166 | |
| 1126 | | | KFD13680.1 | 26728 | 29957 | 1099 | 44083 | 53715 | |
| 1127 | | | ACE91185.1 | 26730 | 29959 | 1074 | 2221342 | 2230688 | |
| 1128 | | | KKZ86595.1 | 26732 | 29961 | 1140 | 262183 | 271712 | |
| 1129 | | | KQU05107.1 | 26734 | 29963 | 1079 | 513 | 9877 | |
| 1130 | | | CCF21880.1 | 26736 | 29965 | 911 | 4095719 | 4104474 | |
| 1131 | | | KQZ63425.1 | 26738 | 29967 | 1079 | 733975 | 743234 | |
| 1132 | | | KIV64826.1 | 26740 | 29969 | 1133 | 375717 | 385216 | |
| 1133 | | | KQB14223.1 | 26742 | 29971 | 1033 | 23685 | 32821 | |
| 1134 | | | KQB14198.1 | 26744 | 29973 | 1033 | 23685 | 32821 | |
| 1135 | | | ETX15682.1 | 26746 | 29975 | 1055 | 1000 | 10241 | |
| 1136 | | | KJS41397.1 | 26748 | 29977 | 1042 | 38507 | 47619 | |
| 1137 | | | EPX84388.1 | 26750 | 29979 | 547 | 176357 | 182232 | |
| 1138 | | | GAS73921.1 | 26752 | 29981 | 1099 | 341435 | 351068 | |
| 1139 | | | GAS79507.1 | 26754 | 29983 | 1099 | 214261 | 223894 | |
| 1140 | | | KHP17886.1 | 26756 | 29985 | 1099 | 348692 | 358324 | |
| 1141 | | | KHP06993.1 | 26758 | 29987 | 1099 | 389798 | 399430 | |
| 1142 | | | ESJ40085.1 | 26760 | 29989 | 1099 | 28 | 9660 | |
| 1143 | | | KNB22032.1 | 26762 | 29991 | 1099 | 41580 | 51212 | |
| 1144 | | | AJD94188.1 | 26764 | 29993 | 1099 | 505155 | 514787 | |
| 1145 | | | AJD98580.1 | 26766 | 29995 | 1099 | 968609 | 978241 | |
| 1146 | | | CEA95106.1 | 26768 | 29997 | 1099 | 1741830 | 1751462 | |
| 1147 | | | CEB50849.1 | 26770 | 29999 | 1099 | 1749103 | 1758735 | |
| 1148 | | | CDU93373.1 | 26772 | 30001 | 1099 | 743440 | 753072 | |
| 1149 | | | CEB74778.1 | 26774 | 30003 | 1099 | 3835521 | 3845153 | |
| 1150 | | | CED02081.1 | 26776 | 30005 | 1099 | 1754122 | 1763754 | |
| 1151 | | | CEC70259.1 | 26778 | 30007 | 1099 | 1753225 | 1762857 | |
| 1152 | | | CDZ07209.1 | 26780 | 30009 | 1099 | 1754686 | 1764318 | |
| 1153 | | | CED89042.1 | 26782 | 30011 | 1099 | 3619879 | 3629511 | |
| 1154 | | | CDV50415.1 | 26784 | 30013 | 1099 | 1753988 | 1763620 | |
| 1155 | | | CFW88484.1 | 26786 | 30015 | 1099 | 309528 | 319160 | |
| 1156 | | | CDZ03620.1 | 26788 | 30017 | 1099 | 2401412 | 2411044 | |
| 1157 | | | CEC95430.1 | 26790 | 30019 | 1099 | 343828 | 353460 | |
| 1158 | | | CDV33413.1 | 26792 | 30021 | 1099 | 1855125 | 1864757 | |
| 1159 | | | CEB60512.1 | 26794 | 30023 | 1099 | 3889568 | 3899200 | |
| 1160 | | | CEC11573.1 | 26796 | 30025 | 1099 | 2654142 | 2663774 | |
| 1161 | | | CEA33006.1 | 26798 | 30027 | 1099 | 607822 | 617454 | |
| 1162 | | | CDV54742.1 | 26800 | 30029 | 1099 | 1754224 | 1763856 | |
| 1163 | | | CEA34475.1 | 26802 | 30031 | 1099 | 2091180 | 2100812 | |
| 1164 | | | CED50261.1 | 26804 | 30033 | 1099 | 676459 | 686091 | |
| 1165 | | | CEC06550.1 | 26806 | 30035 | 1099 | 309528 | 319160 | |
| 1166 | | | CDY81480.1 | 26808 | 30037 | 1099 | 310058 | 319690 | |
| 1167 | | | CEC66464.1 | 26810 | 30039 | 1099 | 3761780 | 3771412 | |
| 1168 | | | CDU75282.1 | 26812 | 30041 | 1099 | 309308 | 318940 | |
| 1169 | | | CDV02408.1 | 26814 | 30043 | 1099 | 642213 | 651845 | |
| 1170 | | | CDU44895.1 | 26816 | 30045 | 1099 | 343655 | 353287 | |
| 1171 | | | CEA82133.1 | 26818 | 30047 | 1099 | 1789537 | 1799169 | |
| 1172 | | | CDV48178.1 | 26820 | 30049 | 1099 | 3855153 | 3864785 | |
| 1173 | | | CEA48951.1 | 26822 | 30051 | 1099 | 344237 | 353869 | |
| 1174 | | | CDU62795.1 | 26824 | 30053 | 1099 | 1785128 | 1794760 | |
| 1175 | | | CDV82107.1 | 26826 | 30055 | 1099 | 2344699 | 2354331 | |
| 1176 | | | CEC15878.1 | 26828 | 30057 | 1099 | 607549 | 617181 | |
| 1177 | | | CEB34163.1 | 26830 | 30059 | 1099 | 1754745 | 1764377 | |
| 1178 | | | CEC22818.1 | 26832 | 30061 | 1099 | 1753415 | 1763047 | |
| 1179 | | | CEC29447.1 | 26834 | 30063 | 1099 | 1962606 | 1972238 | |
| 1180 | | | CEC81193.1 | 26836 | 30065 | 1099 | 1752823 | 1762455 | |
| 1181 | | | CEC27085.1 | 26838 | 30067 | 1099 | 643261 | 652893 | |
| 1182 | | | CDV36505.1 | 26840 | 30069 | 1099 | 3830845 | 3840477 | |
| 1183 | | | CDV99881.1 | 26842 | 30071 | 1099 | 1788948 | 1798580 | |
| 1184 | | | CEB21254.1 | 26844 | 30073 | 1099 | 1785448 | 1795080 | |
| 1185 | | | CED29540.1 | 26846 | 30075 | 1099 | 2414784 | 2424416 | |
| 1186 | | | CEA65654.1 | 26848 | 30077 | 1099 | 2411587 | 2421219 | |
| 1187 | | | CDV77398.1 | 26850 | 30079 | 1099 | 1754018 | 1763650 | |
| 1188 | | | CEB99329.1 | 26852 | 30081 | 1099 | 666458 | 676090 | |
| 1189 | | | CEC87093.1 | 26854 | 30083 | 1099 | 102895 | 112527 | |
| 1190 | | | CEC03343.1 | 26856 | 30085 | 1099 | 2038905 | 2048537 | |
| 1191 | | | CDW68452.1 | 26858 | 30087 | 1099 | 1754895 | 1764527 | |
| 1192 | | | CEB36542.1 | 26860 | 30089 | 1099 | 1505242 | 1514874 | |
| 1193 | | | CEB92481.1 | 26862 | 30091 | 1099 | 1754998 | 1764630 | |
| 1194 | | | CEC70640.1 | 26864 | 30093 | 1099 | 2655621 | 2665253 | |
| 1195 | | | CED19421.1 | 26866 | 30095 | 1099 | 309298 | 318930 | |
| 1196 | | | CDU57399.1 | 26868 | 30097 | 1099 | 1744361 | 1753993 | |
| 1197 | | | CEC55612.1 | 26870 | 30099 | 1099 | 3432850 | 3442482 | |
| 1198 | | | CDW10772.1 | 26872 | 30101 | 1099 | 1775102 | 1784734 | |
| 1199 | | | CED40437.1 | 26874 | 30103 | 1099 | 309601 | 319233 | |
| 1200 | | | CEA77531.1 | 26876 | 30105 | 1099 | 344224 | 353856 | |
| 1201 | | | CEC78581.1 | 26878 | 30107 | 1099 | 1754570 | 1764202 | |
| 1202 | | | CDV87320.1 | 26880 | 30109 | 1099 | 1754358 | 1763990 | |
| 1203 | | | CEA60264.1 | 26882 | 30111 | 1099 | 2553269 | 2562901 | |
| 1204 | | | CDU45979.1 | 26884 | 30113 | 1099 | 1753121 | 1762753 | |
| 1205 | | | CED26178.1 | 26886 | 30115 | 1099 | 1854653 | 1864285 | |
| 1206 | | | CEA55546.1 | 26888 | 30117 | 1099 | 1754980 | 1764612 | |
| 1207 | | | CDU57361.1 | 26890 | 30119 | 1099 | 1753795 | 1763427 | |
| 1208 | | | CEC22628.1 | 26892 | 30121 | 1099 | 309060 | 318692 | |
| 1209 | | | CDU81244.1 | 26894 | 30123 | 1099 | 2357174 | 2366806 | |
| 1210 | | | CEC22208.1 | 26896 | 30125 | 1099 | 1746204 | 1755836 | |
| 1211 | | | CDV00141.1 | 26898 | 30127 | 1099 | 1753396 | 1763028 | |
| 1212 | | | CDV32910.1 | 26900 | 30129 | 1099 | 309248 | 318880 | |
| 1213 | | | CEB32348.1 | 26902 | 30131 | 1099 | 1787540 | 1797172 | |
| 1214 | | | CDU66161.1 | 26904 | 30133 | 1099 | 134848 | 144480 | |
| 1215 | | | CEB23433.1 | 26906 | 30135 | 1099 | 1754743 | 1764375 | |
| 1216 | | | CEA69989.1 | 26908 | 30137 | 1099 | 1788795 | 1798427 | |
| 1217 | | | CED34531.1 | 26910 | 30139 | 1099 | 1762471 | 1772103 | |
| 1218 | | | CDU60158.1 | 26912 | 30141 | 1099 | 1772870 | 1782502 | |
| 1219 | | | CEB02153.1 | 26914 | 30143 | 1099 | 1505400 | 1515032 | |
| 1220 | | | CDV16495.1 | 26916 | 30145 | 1099 | 1753390 | 1763022 | |
| 1221 | | | CEA70649.1 | 26918 | 30147 | 1099 | 309354 | 318986 | |
| 1222 | | | CEB86473.1 | 26920 | 30149 | 1099 | 3281543 | 3291175 | |
| 1223 | | | CDV63744.1 | 26922 | 30151 | 1099 | 1753068 | 1762700 | |
| 1224 | | | CEB22320.1 | 26924 | 30153 | 1099 | 1298956 | 1308588 | |
| 1225 | | | CDY85019.1 | 26926 | 30155 | 1099 | 633632 | 643264 | |
| 1226 | | | CDY91993.1 | 26928 | 30157 | 1099 | 2414151 | 2423783 | |
| 1227 | | | CDV63674.1 | 26930 | 30159 | 1099 | 601562 | 611194 | |
| 1228 | | | CDV76963.1 | 26932 | 30161 | 1099 | 1298539 | 1308171 | |
| 1229 | | | CDV25168.1 | 26934 | 30163 | 1099 | 77424 | 87056 | |
| 1230 | | | CEA29073.1 | 26936 | 30165 | 1099 | 3746810 | 3756442 | |
| 1231 | | | CEC16657.1 | 26938 | 30167 | 1099 | 1743338 | 1752970 | |
| 1232 | | | CDV58583.1 | 26940 | 30169 | 1099 | 1753914 | 1763546 | |
| 1233 | | | CDV98720.1 | 26942 | 30171 | 1099 | 601239 | 610871 | |
| 1234 | | | CED02856.1 | 26944 | 30173 | 1099 | 2381174 | 2390806 | |
| 1235 | | | CDU96713.1 | 26946 | 30175 | 1099 | 642929 | 652561 | |
| 1236 | | | CEB76242.1 | 26948 | 30177 | 1099 | 3766799 | 3776431 | |
| 1237 | | | CEC37126.1 | 26950 | 30179 | 1099 | 597747 | 607379 | |
| 1238 | | | CDU99405.1 | 26952 | 30181 | 1099 | 1754390 | 1764022 | |
| 1239 | | | CEB54820.1 | 26954 | 30183 | 1099 | 1847245 | 1856877 | |
| 1240 | | | CEC86157.1 | 26956 | 30185 | 1099 | 165417 | 175049 | |
| 1241 | | | CDV41201.1 | 26958 | 30187 | 1099 | 608177 | 617809 | |
| 1242 | | | CDV19348.1 | 26960 | 30189 | 1099 | 608193 | 617825 | |
| 1243 | | | CEA51462.1 | 26962 | 30191 | 1099 | 1785262 | 1794894 | |
| 1244 | | | CEA31323.1 | 26964 | 30193 | 1099 | 669961 | 679593 | |
| 1245 | | | CEC70731.1 | 26966 | 30195 | 1099 | 1753358 | 1762990 | |
| 1246 | | | CEB55411.1 | 26968 | 30197 | 1099 | 1749599 | 1759231 | |
| 1247 | | | CED14443.1 | 26970 | 30199 | 1099 | 1754022 | 1763654 | |
| 1248 | | | CED18826.1 | 26972 | 30201 | 1099 | 607130 | 616762 | |
| 1249 | | | CEB11072.1 | 26974 | 30203 | 1099 | 1753867 | 1763499 | |
| 1250 | | | CEA27402.1 | 26976 | 30205 | 1099 | 3814476 | 3824108 | |
| 1251 | | | CED08653.1 | 26978 | 30207 | 1099 | 668385 | 678017 | |
| 1252 | | | CEB93599.1 | 26980 | 30209 | 1099 | 2227341 | 2236973 | |
| 1253 | | | CDY97807.1 | 26982 | 30211 | 1099 | 706460 | 716092 | |
| 1254 | | | CEA81540.1 | 26984 | 30213 | 1099 | 309301 | 318933 | |
| 1255 | | | CEA95806.1 | 26986 | 30215 | 1099 | 309463 | 319095 | |
| 1256 | | | CED39170.1 | 26988 | 30217 | 1099 | 2443048 | 2452680 | |
| 1257 | | | CEC46310.1 | 26990 | 30219 | 1099 | 3244245 | 3253877 | |
| 1258 | | | CEB93178.1 | 26992 | 30221 | 1099 | 517816 | 527448 | |
| 1259 | | | CEB06236.1 | 26994 | 30223 | 1099 | 2332565 | 2342197 | |
| 1260 | | | CEC59901.1 | 26996 | 30225 | 1099 | 1788361 | 1797993 | |
| 1261 | | | CDW11872.1 | 26998 | 30227 | 1099 | 1789272 | 1798904 | |
| 1262 | | | CEB87182.1 | 27000 | 30229 | 1099 | 1777428 | 1787060 | |
| 1263 | | | CDU53208.1 | 27002 | 30231 | 1099 | 503202 | 512834 | |
| 1264 | | | CEB57391.1 | 27004 | 30233 | 1099 | 676605 | 686237 | |
| 1265 | | | CED48958.1 | 27006 | 30235 | 1099 | 3782745 | 3792377 | |
| 1266 | | | CDW00039.1 | 27008 | 30237 | 1099 | 1754437 | 1764069 | |
| 1267 | | | CDV01787.1 | 27010 | 30239 | 1099 | 1788427 | 1798059 | |
| 1268 | | | CEA40519.1 | 27012 | 30241 | 1099 | 1783442 | 1793074 | |
| 1269 | | | CEB13673.1 | 27014 | 30243 | 1099 | 1753084 | 1762716 | |
| 1270 | | | CEA18908.1 | 27016 | 30245 | 1099 | 1854428 | 1864060 | |
| 1271 | | | CDY92812.1 | 27018 | 30247 | 1099 | 1752529 | 1762161 | |
| 1272 | | | CEA88154.1 | 27020 | 30249 | 1099 | 1506435 | 1516067 | |
| 1273 | | | AKO82198.1 | 27022 | 30251 | 1099 | 4482333 | 4491965 | |
| 1274 | | | CAR62301.1 | 27024 | 30253 | 1099 | 4477563 | 4487195 | |
| 1275 | | | ESF93195.1 | 27026 | 30255 | 1099 | 11502 | 21134 | |
| 1276 | | | AAV80043.1 | 27028 | 30257 | 1099 | 4482333 | 4491965 | |
| 1277 | | | EPE40531.1 | 27030 | 30259 | 1099 | 85913 | 95545 | |
| 1278 | | | EPE40313.1 | 27032 | 30261 | 1099 | 85780 | 95412 | |
| 1279 | | | EPE40095.1 | 27034 | 30263 | 1099 | 85905 | 95537 | |
| 1280 | | | KSB43364.1 | 27036 | 30265 | 1099 | 156994 | 166626 | |
| 1281 | | | ELX27349.1 | 27038 | 30267 | 1099 | 65113 | 74745 | |
| 1282 | | | EHC78170.1 | 27040 | 30269 | 77 | 221 | 6787 | |
| 1283 | | | ESF47593.1 | 27042 | 30271 | 1099 | 172275 | 181907 | |
| 1284 | | | CUS01662.1 | 27044 | 30273 | 1099 | 4951384 | 4961016 | |
| 1285 | | | CUS00367.1 | 27046 | 30275 | 944 | 4759655 | 4768822 | |
| 1286 | | | CUS00368.1 | 27048 | 30277 | 944 | 4851124 | 4860291 | |
| 1287 | | | CUS00366.1 | 27050 | 30279 | 1099 | 5018293 | 5027925 | |
| 1288 | | | CBY96200.1 | 27052 | 30281 | 944 | 85 | 9252 | |
| 1289 | | | EDZ29008.1 | 27054 | 30283 | 285 | 63002 | 70192 | |
| 1290 | | | KNM15296.1 | 27056 | 30285 | 1099 | 282369 | 292001 | |
| 1291 | | | KNM69141.1 | 27058 | 30287 | 1099 | 343834 | 353466 | |
| 1292 | | | KNO58855.1 | 27060 | 30289 | 1099 | 206734 | 216366 | |
| 1293 | | | KNW78916.1 | 27062 | 30291 | 1099 | 1907 | 11539 | |
| 1294 | | | KTZ77424.1 | 27064 | 30293 | 1099 | 96532 | 106164 | |
| 1295 | | | KUC23740.1 | 27066 | 30295 | 1099 | 171297 | 180929 | |
| 1296 | | | ESF03067.1 | 27068 | 30297 | 1099 | 120111 | 129743 | |
| 1297 | | | KMJ09050.1 | 27070 | 30299 | 1099 | 529831 | 539463 | |
| 1298 | | | CVG51486.1 | 27072 | 30301 | 1099 | 238999 | 248631 | |
| 1299 | | | CVB32240.1 | 27074 | 30303 | 1099 | 239061 | 248693 | |
| 1300 | | | EZQ69786.1 | 27076 | 30305 | 1099 | 90156 | 99788 | |
| 1301 | | | EFE97639.1 | 27078 | 30307 | 482 | 743953 | 751819 | |
| 1302 | | | KFK98827.1 | 27080 | 30309 | 1095 | 286734 | 296378 | |
| 1303 | | | KFK92330.1 | 27082 | 30311 | 1095 | 1654 | 11298 | |
| 1304 | | | ABK48268.1 | 27084 | 30313 | 1180 | 2437310 | 2447236 | |
| 1305 | | | AFJ48529.1 | 27086 | 30315 | 263 | 3585169 | 3592293 | |
| 1306 | | | GAB81434.1 | 27088 | 30317 | 269 | 178785 | 185928 | |
| 1307 | | | ACP25984.1 | 27090 | 30319 | 1083 | 2298324 | 2307700 | |
| 1308 | | | CAN98361.1 | 27092 | 30321 | 1152 | 11387949 | 11397690 | |
| 1309 | | | EPR18397.1 | 27094 | 30323 | 1038 | 2646 | 9729 | |
| 1310 | | | KKW93258.1 | 27096 | 30325 | 1099 | 135724 | 144994 | |
| 1311 | | | CCW17774.1 | 27098 | 30327 | 1098 | 7698 | 15633 | |
| 1312 | | | KER36315.1 | 27100 | 30329 | 1064 | 2812 | 11959 | |
| 1313 | | | KQM97529.1 | 27102 | 30331 | 1002 | 1512 | 10520 | |
| 1314 | | | KPV39945.1 | 27104 | 30333 | 499 | 46068 | 53818 | |
| 1315 | | | KQW61500.1 | 27106 | 30335 | 1187 | 77355 | 87231 | |
| 1316 | | | KLE23445.1 | 27108 | 30337 | 1121 | 177407 | 186987 | |
| 1317 | | | EDL70290.1 | 27110 | 30339 | 1125 | 191775 | 201360 | |
| 1318 | | | KHD25448.1 | 27112 | 30341 | 1169 | 189282 | 199238 | |
| 1319 | | | KFE26106.1 | 27114 | 30343 | 1118 | 33695 | 43271 | |
| 1320 | | | KFD94236.1 | 27116 | 30345 | 1118 | 12302 | 21878 | |
| 1321 | | | EJH60536.1 | 27118 | 30347 | 1102 | 68538 | 78157 | |
| 1322 | | | EMQ54781.1 | 27120 | 30349 | 1118 | 244112 | 253688 | |
| 1323 | | | KNA52261.1 | 27122 | 30351 | 1118 | 12538 | 22114 | |
| 1324 | | | GAD79747.1 | 27124 | 30353 | 1169 | 25836 | 35781 | |
| 1325 | | | KNY42356.1 | 27126 | 30355 | 1125 | 111138 | 120729 | |
| 1326 | | | KHF16001.1 | 27128 | 30357 | 1121 | 3364 | 12944 | |
| 1327 | | | KOF25302.1 | 27130 | 30359 | 1121 | 11973 | 21559 | |
| 1328 | | | KHT39220.1 | 27132 | 30361 | 1102 | 105669 | 115288 | |
| 1329 | | | KYN85118.1 | 27134 | 30363 | 1118 | 68509 | 78085 | |
| 1330 | | | EKM24798.1 | 27136 | 30365 | 1169 | 448059 | 458009 | |
| 1331 | | | CDU08195.1 | 27138 | 30367 | 1102 | 291331 | 300963 | |
| 1332 | | | CAV18547.1 | 27140 | 30369 | 1124 | 1498484 | 1508125 | |
| 1333 | | | KWU01491.1 | 27142 | 30371 | 1119 | 60437 | 70011 | |
| 1334 | | | KOO13423.1 | 27144 | 30373 | 1125 | 88330 | 97921 | |
| 1335 | | | CNI05770.1 | 27146 | 30375 | 1100 | 395449 | 405084 | |
| 1336 | | | CQJ58416.1 | 27148 | 30377 | 1100 | 455230 | 464865 | |
| 1337 | | | CQJ21073.1 | 27150 | 30379 | 1117 | 22700 | 32382 | |
| 1338 | | | CFR01913.1 | 27152 | 30381 | 1100 | 235368 | 245003 | |
| 1339 | | | CNH31670.1 | 27154 | 30383 | 1100 | 474426 | 484061 | |
| 1340 | | | CFV22965.1 | 27156 | 30385 | 1100 | 113536 | 123171 | |
| 1341 | | | KFE39719.1 | 27158 | 30387 | 1100 | 4143 | 13778 | |
| 1342 | | | AKA39097.1 | 27160 | 30389 | 1100 | 2710693 | 2720328 | |
| 1343 | | | EHM51571.1 | 27162 | 30391 | 944 | 252227 | 261394 | |

### Example 7

### Genomic Identification and Analysis of an Anti-Plasmid System

Objective:

Some of the putative defense systems experimentally tested did not show any anti-phage activity despite being strongly associated with known defense genes. It was reasoned that some of these systems may defend against other forms of foreign nucleic acid. The objective in this case was to analyze a candidate defense system, which we hereby denote Wadjet (god protector of ancient Egypt) for further experimentation.

Methods:

To test this hypothesis, the Wadjet system was selected for further experimentation. The selected candidate system is a 4-gene system, jetA, jetB, jetC, and jetC, that is common in microbial genomes and is very frequently found next to defense genes (FIG. 7A). Three different types of the Wadjet system were cloned from three separate Bacillus species into B. subtilis BEST7003 (FIG. 7B).

Results:

While none of these systems provided protection against any of the 10 Bacillus phages in the array tested (data not shown), all three consistently and significantly reduced transformation efficiency of the episomal plasmid pHCMC05 (FIG. 7C). These results suggest that this may be a defense system specifically targeting foreign plasmids.

Three different domain compositions were identified, each encoding a different set of pfams, but all with common sequence signatures marking them as three types of Wadjet (FIG. 7B). While the pfam domains of Wadjet genes are mostly defined as “domains of unknown function”, structural modeling using Phyre2 showed structural homology between JetA, JetB and JetC and genes belonging to the housekeeping condensin system MukF, MukE and MukB, respectively. Bacterial condensins are chromosome-organizing complexes that are responsible for DNA condensation and accurate segregation during replication, and mutations in the housekeeping condensins lead to severe defects in chromosome segregation and viability. Several versions of housekeeping codensins appear in bacterial genomes: SMC, MukBEF and MksBEF; the Wadjet system was previously noted as a distant homolog of the MksBEF system described in P. aeruginosa.

While the domain organization of the jetABC genes may lead to the hypothesis that Wadjet is an alternative condensin system involved in bacterial chromosome maintenance, the data herein presented imply that its role is defensive. This system is highly enriched within defense islands, undergoes extensive horizontal gene transfer, and is only sporadically found within strains of the same species, all of which is inconsistent with a core, essential role in chromosome maintenance. The Wadjet system may have adapted from a MukBEF condensin ancestor to become a defense system. Possibly, the system identifies foreign plasmids and uses its condensin properties to interfere with proper plasmid segregation into daughter cells. Notably, plasmid transformation in B. subtilis takes place via the natural competence of this organism, during which the plasmid DNA is transformed to the cell through dedicated transporters as single-stranded DNA (ssDNA). It is possible that the Wadjet system protects against rampant natural transformation or, alternatively, may specifically target ssDNA phages. However, as no ssDNA phage was reported for B. subtilis, it could not be tested whether ssDNA phages are specifically blocked by the Wadjet systems we cloned in B. subtilis BEST7003.

The Wadjet system is broadly spread in bacterial and archaeal genomes (found in ˜6% of the genomes that were studied), where it presents high sequence diversity (Table 11; FIG. 3H). Deletion of each of the four genes in Type I Wadjet from B. cereus Q1 abolished its activity and restored plasmid transformation, indicating that each of the genes is essential for anti-plasmid defense (FIG. 7C). Moreover, point mutations E59K/K60E in JetB, predicted to disrupt the MukE-MukF-like protein-protein interactions, resulted in loss of protective activity against plasmids (data not shown), and so has the E1025Q mutation in the Walker-B motif of JetC that is predicted to abolish ATPase activity. The JetD gene, which has no homology to genes in the Muk system has a putative topoisomerase VI domain based on structural predictions; a point mutation JetD:E226A, predicted to diminish binding of the topoisomerase VI domain to DNA, also abolished the protective activity of the system.

Table 11: The Wadjet Defense System and Components Thereof

This table is presented in landscape orientation. Due to the number of columns, the table has been split into parts. Part A includes the first set of columns and therefore should be place to the left of subsequent parts (Part B and where relevant Part C). To avoid any doubt, the rows of the table in each part line up once the tables are placed together.

Lengthy table referenced here
US20200109395A1-20200409-T00001
Please refer to the end of the specification for access instructions.

### Example 8

### Genomic Identification and Analysis of Additional Defense System

Objective:

To identify additional Defense Systems

Methods:

As above for Examples 1-7, wherein anchor protein families used in the searches were as follows:

for the Hachiman Family: pfam08878;

for the Gabija Family: pfam13175, pfam13304;

for the Kiwa Family: pfam16162;

for the Lamassu Family: pfam14130;

for the Shedu Family: pfam14082;

for the Septu Family: COG3950; and

for the Wadjet Family: COG4913, COG4924, pfam11796, pfam09664, pfam13555.

Results:

each of these systems was validated by introducing the system constructs into an appropriate bacterial host. The Hachiman, Shedu, Gabija, Septu, and Lamassu candidate Defense Systems were each individually engineered into B. subtilis BEST7003, which does not contain the respective defense systems identified. The correct insertion of the system into the B. subtilis BEST7003 genome was verified by whole genome sequencing. The defense system containing B. subtilis BEST7003 strains were challenged with multiple phages spanning the three families Myoviridae, Siphoviridae, and Podoviridae. The cloned systems were found to confer defense against myophages (FIG. 3B), with different fold protection to all or a subset of the phage provided as shown.

The Kiwa candidate Defense System was engineered into a host E. coli (E. coli MG1655; correct insertion checked by whole genome sequencing), wherein it was shown to provide protection against viruses in the Siphoviridae family (FIG. 3C).

The Hachiman, Shedu, Gabija, Septu, and Lamassu candidate Defense Systems were broadly distributed in bacteria and archaea (Tables 12-17; FIG. 3H).

Lengthy table referenced here
US20200109395A1-20200409-T00002
Please refer to the end of the specification for access instructions.

Lengthy table referenced here
US20200109395A1-20200409-T00003
Please refer to the end of the specification for access instructions.

Lengthy table referenced here
US20200109395A1-20200409-T00004
Please refer to the end of the specification for access instructions.

Lengthy table referenced here
US20200109395A1-20200409-T00005
Please refer to the end of the specification for access instructions.

Lengthy table referenced here
US20200109395A1-20200409-T00006
Please refer to the end of the specification for access instructions.

Lengthy table referenced here
US20200109395A1-20200409-T00007
Please refer to the end of the specification for access instructions.

### Summary Examples 1-8

Examples 1-8 significantly expand the known arsenal of defense systems used by prokaryotes for protection against phages. This knowledge leads to broader understanding of the global defense capabilities of bacteria, and indeed with our results the content of defense islands now becomes much clearer (FIGS. 4A, 4D, 5A, 6A, 7A). However, the results do not yet expose the complete set of prokaryotic defense systems. Out of the 28 candidate systems tested, 10 were verified as anti-phage defense systems and an additional one showed protection against plasmids. The remaining 17, although not verified by the experiments, do not necessarily represent false predictions, as exemplified by the fact that only 50% of our positive control systems showed defense in the assays. Lack of activity could possibly stem from incompatibility of some tested systems with the recipient organism (E. coli or B. subtilis), or due to pseudogenization of some systems in their genome of origin. Some systems can be highly specific against a certain type of phages or foreign genetic element not represented in the phage set, while others may work in a specific condition not tested in the study. Clade-specific potential systems such as those found only in archaea or cyanobacteria (Table 3) were not tested in this study and can represent a more specialized defense arsenal unique only to a subset of organisms. Finally, some true systems may have been missed by falsely tagging them as belonging to the “mobilome” (Table 2), as mobile genetic elements have intimate evolutionary relationship with defense systems.

While certain features have been illustrated and described herein, many modifications, substitutions, changes, and equivalents will now occur to those of ordinary skill in the art. It is, therefore, to be understood that the appended claims are intended to cover all such modifications and changes as fall within the true spirit of the disclosure presented herein.

LENGTHY TABLES
The patent application contains a lengthy table section. A copy of the table is available in electronic form from the USPTO web site (http://seqdata.uspto.gov/?pageRequest=docDetail&DocID=US20200109395A1). An electronic copy of the table will also be available from the USPTO upon request and payment of the fee set forth in 37 CFR 1.19(b)(3).

What is claimed is:

1. A method of protecting bacteria from foreign nucleic acid invasion, said method comprising a step of introducing into the bacteria at least one defense system (a) to (o), said at least one defense system comprising:
(a) a Defense System IV comprising at least two different polypeptide components selected from
a HamA polypeptide comprising a pfam08878 domain or a DUF1837 domain or a combination thereof, or comprising an amino acid sequence having at least 80% identity to a sequence selected from the group referenced in Table 12 rows 2-1782 columns H and I; and
a HamB polypeptide comprising COG1204 domain or a pfam00270 domain or a pfam00271 domain or any combination thereof, or comprising an amino acid sequence having at least 80% identity to a sequence selected from the group referenced in Table 12 rows 2-1782 columns L and M; or
(b) a Defense System Ia comprising at least two different polypeptide components selected from
a ZorA polypeptide comprising a pfam01618 domain, or comprising an amino acid sequence having at least 80% identity to a sequence selected from the group referenced in Table 8 rows 2-1830 columns J and K;
a ZorB polypeptide comprising a pfam13677 domain or a pfam00691 domain or a combination thereof, or comprising an amino acid sequence having at least 80% identity to a sequence selected from the group referenced in Table 8 rows 2-1830 columns N and O;
a ZorC polypeptide comprising a pfam15611 domain, or comprising an amino acid sequence having at least 80% identity to a sequence selected from the group referenced in Table 8 rows 2-1174 columns R and S; and
a ZorD polypeptide comprising a pfam00176 domain or a pfam00271 domain or a combination thereof, or comprising an amino acid sequence having at least 80% identity to a sequence selected from the group referenced in Table 8 rows 2-1174 columns V and W;
or
(c) a Defense System Ib comprising a ZorE polypeptide comprising a pfam01844 domain or a COG3183 domain or a combination thereof, or comprising an amino acid sequence having at least 80% identity to a sequence selected from the group referenced in Table 8 rows 1175-1830 columns Z and AA; and at least one other polypeptide component selected from
a ZorA polypeptide comprising a pfam01618 domain, or comprising an amino acid sequence having at least 80% identity to a sequence selected from the group referenced in Table 8 rows 2-1830 columns J and K; and
a ZorB polypeptide comprising a pfam13677 domain or a pfam00691 domain or a combination thereof, or comprising an amino acid sequence having at leak 80% identity to a sequence selected from the group referenced in Table 8 rows 2-1830 columns N and O; or
(d) a Defense System II comprising at least two different polypeptide components selected from
a ThsA polypeptide comprising a pfam13289 domain or a pfam14519 domain or a combination thereof, or comprising an amino acid sequence having at least 80% identity to a sequence selected from the group referenced in Table 9 rows 2-2100 columns I and J; and
a ThsB polypeptide comprising a pfam13676 domain or a pfam08937 domain or a pfam08357 domain or any combination thereof, or comprising an amino acid sequence having at least 80% identity to a sequence selected from the group referenced in Table 9 rows 2-2100 columns M and N, Q and R, U and V, Y and Z, and AC and AD; or
(e) a Defense System IIIa comprising at least two different polypeptide components selected from
a DruA polypeptide comprising a DUF4338 domain or a pfam14236 domain or a combination thereof; or comprising an amino acid sequence having at least 80% identity to a sequence selected from the group referenced in Table 10 rows 2-123 columns J and K, or a combination thereof;
a DruB polypeptide comprising an amino acid sequence having at least 80% identity to a sequence selected from the group referenced in Table 10 rows 2-123 columns N and O;
a DruC polypeptide comprising an amino acid sequence having at least 80% identity to a sequence selected from the group referenced in Table 10 rows 2-123 columns R and S;
a DruD polypeptide comprising an amino acid sequence having at least 80% identity to a sequence selected from the group referenced in Table 10 rows 2-123 columns V and W; and
a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA; or
(f) a Defense System IIIb comprising at least two different polypeptide components selected from
a DruM polypeptide comprising a pfam00145 domain or a COG0270 domain or a combination thereof, or comprising an amino acid sequence having at least 80% identity to a sequence selected from the group referenced in Table 10 rows 124-295 columns AD and AE;
a DruF polypeptide comprising an amino acid sequence having at least 80% identity to a sequence selected from the group referenced in Table 10 rows 124-295 columns AH and AI;
a DruG polypeptide comprising an amino acid sequence having at least 80% identity to a sequence selected from the group referenced in Table 10 rows 124-295 columns AL and AM; and
a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 column Z and AA; or
(g) a Defense System IIIc comprising at least two different polypeptide components selected from
a DruH polypeptide comprising an amino acid sequence having at least 80% identity to a sequence selected from the group referenced in Table 10 rows 296-1343 columns AP and AQ; and
a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA; or
(h) a Defense System V comprising a SduA polypeptide comprising a pfam14082 domain or a pfam01939 domain or a combination thereof, or comprising an amino acid sequence having at least 80% identity to a sequence selected from the group referenced in Table 13 rows 2-1247 columns H and I; or
(i) a Defense System VI comprising at least two different polypeptide components selected from
a GajA polypeptide comprising a pfam13175 domain or a COG3593 domain or a combination thereof, or comprising an amino acid sequence having at least 80% identity to a sequence selected from the group referenced in Table 14 rows 2-4599 columns H and I; and
a GajB polypeptide comprising a pfam04257 domain or a pfam00580 domain or a pfam13361 domain or any combination thereof, or comprising an amino acid sequence having at least 80% identity to a sequence selected from the group referenced in Table 14 rows 2-4599 columns L and M; or
(j) a Defense System VII comprising at least two different polypeptide components selected from
a PtuA polypeptide comprising a pfam13304 domain or a pfam02463 domain or a combination thereof, or comprising an amino acid sequence having at least 80% identity to a sequence selected from the group referenced in Table 15 rows 2-12507 columns H and I; and
a PtuB polypeptide comprising pfam13395 domain or a pfam 01844 domain or a combination thereof, or comprising an amino acid sequence having at least 80% identity to a sequence selected from the group referenced in Table 15 rows 2-2507 columns L and M; or
(k) a Defense System VIII comprising at least two different polypeptide components selected from
a LmuA polypeptide comprising a pfam14130 domain or a DUF4297 domain or a combination thereof, or comprising an amino acid sequence having at least 80% identity to a sequence selected from the group referenced in Table 16 rows 2-698 columns H and I; and
a LmuB polypeptide comprising a pfam02463 domain, or comprising an amino acid sequence having at least 80% identity to a sequence selected from the group referenced in Table 16 rows 2-698 columns L and M; or
(l) a Defense System IX comprising at least two different polypeptide components selected from
a KwaA polypeptide comprising an amino acid sequence having at least 80% identity to a sequence selected from the group referenced in Table 17 rows 2-935 columns H and I; and
a KwaB polypeptide comprising a pfam16162 domain or a DUF4868 domain or a combination thereof; or comprising an amino acid sequence having at least 80% identity to a sequence selected from the group referenced in Table 17 rows 2-935 columns L and M; or
(m) a Defense System Xa comprising at least two different polypeptide components selected from
a JetA polypeptide comprising a pfam11855 domain or a DUF3375 domain or a combination thereof, or comprising an amino acid sequence having at least 80% identity to a sequence selected from the group referenced in Table 11 rows 2-2322 columns J and K;
a JetB polypeptide comprising a pfam13835 domain or a DUF4194 domain or a combination thereof, or comprising an amino acid sequence having at least 80% identity to a sequence selected from the group referenced in Table 11 rows 2-2322 columns N and O;
a JetC polypeptide comprising a pfam13555 domain or a pfam13558 domain or a COG4913 domain or any combination thereof; or comprising an amino acid sequence having at least 80% identity to a sequence selected from the group referenced in Table 11 rows 2-2322 columns R and S; and
a JetD polypeptide comprising a pfam11795 domain or a DUF3322 domain or a pfam09983 domain or a DUF2220 domain or a COG4924 domain or any combination thereof, or comprising an amino acid sequence having at least 80% identity to a sequence selected from the group referenced in Table 11 rows 2-2322 columns V and W; or
(n) a Defense System Xb comprising at least two different polypeptide components selected from
a JetAⁱⁱ polypeptide comprising a pfam09660 domain or a DUF2397 domain or a combination thereof, or comprising an amino acid sequence having at least 80% identity to a sequence selected from the group referenced in Table 11 rows 2323-2844 columns J and K;
a JetBⁱⁱ polypeptide comprising a pfam09661 domain or a DUF2398 domain or a combination thereof, or comprising an amino acid sequence having at least 80% identity to a sequence selected from the group referenced in Table 11 rows 2323-2844 columns N and O;
a JetCⁱⁱ polypeptide comprising a pfam13558 domain, or comprising an amino acid sequence having at least 80% identity to a sequence selected from the group referenced in Table 11 rows 2323-2844 columns R and S; and
a JetDⁱⁱ polypeptide comprising a pfam11796 domain or a DUF3323 domain or a pfam09664 domain or a DUF2399 domain or a combination thereof, or comprising an amino acid sequence having at least 80% identity to a sequence selected from the group referenced in Table 11 rows 2323-2844 columns V and W; or
(o) a Defense System Xc comprising at least two different polypeptide components selected from
a JetAⁱⁱⁱ polypeptide comprising an amino acid sequence having at least 80% identity to a sequence selected from the group referenced in Table 11 rows 2845-3174 columns and K;
a JetBⁱⁱⁱ polypeptide comprising an amino acid sequence having at least 80% identity to a sequence selected from the group referenced in Table 11 rows 2845-3174 columns N and O;
a JetCⁱⁱⁱ polypeptide comprising a COG1196 domain or comprising an amino acid sequence having at least 80% identity to a sequence selected from the group referenced in Table 11 rows 2845-3174 columns R and S; and
a JetDⁱⁱⁱ polypeptide comprising a pfam09983 domain or a pfam09664 domain or a DUF2220 or a DUF2399 domain or any combination thereof, or comprising an amino acid sequence having at least 80% identity to a sequence selected from the group referenced in Table 11 rows 2845-3174 columns V and W; or
(p) any combination of Defense Systems Ia-Xc (a)-(o);
wherein the introduction of said at least one defense system results in the bacteria being protected from foreign DNA invasion.

2. The method of claim 1, wherein said protecting bacteria from foreign nucleic acid invasion comprises protecting from phage infection, protection from plasmid transformation, or protecting from entry of conjugative elements, or any combination thereof.

3. The method of claim 2, wherein said phage comprises a single stranded DNA (ssDNA) phage, a double stranded DNA (dsDNA) phage, a single stranded RNA (ssRNA) phage, a double stranded RNA (dsRNA) phage, a lytic phage, or a lysogenic phage, or a combination thereof.

4. The method of claim 1, wherein said at least one defense system comprises
(a) a Defense System IV comprising the HamA polypeptide and the HamB polypeptide; or
(b) a Defense System Ia comprising the ZorA polypeptide, the ZorB polypeptide, the ZorC polypeptide, and the ZorD polypeptide; or
(c) a Defense System Ib comprising the ZorA polypeptide, the ZorB polypeptide, and the ZorE polypeptide; or
(d) a Defense System II comprising the ThsA polypeptide, and the ThsB polypeptide; or
(e) a Defense System IIIa comprising the DruA polypeptide, the DruB polypeptide, the DruC polypeptide, the DruD polypeptide, and the DruE polypeptide; or
(f) a Defense System IIIb comprising the DruM polypeptide, the DruF polypeptide, the DruG polypeptide, and the DruE polypeptide; or
(g) a Defense System IIIc comprising the DruH polypeptide and the DruE polypeptide; or
(h) a Defense System V comprising the SduA polypeptide; or
(i) a Defense System VI comprising the GajA polypeptide and the GajB polypeptide; or
(j) a Defense System VII comprising the PtuA polypeptide and the PtuB polypeptide; or
(k) a Defense System VIII comprising the LmuA polypeptide and the LmuB polypeptide; or
(l) a Defense System IX comprising the KwaA polypeptide and the KwaB polypeptide; or
(m) a Defense System Xa comprising the JetA polypeptide, the JetB polypeptide, the JetC polypeptide, and the JetD polypeptide; or
(n) a Defense System Xb comprising the JetAⁱⁱ polypeptide, the JetBⁱⁱ polypeptide, the JetCⁱⁱ polypeptide, and the JetDⁱⁱ polypeptide; or
(o) a Defense System Xc comprising the JetAⁱⁱⁱ polypeptide, the JetBⁱⁱⁱ polypeptide, the JetCⁱⁱⁱ polypeptide, and the JetDⁱⁱⁱ polypeptide; or
(p) any combination of Defense Systems Ia-Xc (a)-(o).

5. The method of claim 1, wherein
(a) said Defense System IV is encoded by
a nucleic acid sequence selected from the group referenced in Table 12, rows 2-1782, columns G, P, and Q or the nucleic acid sequence set forth in SEQ ID NO: 4; or
a nucleic acid sequence encoding the at least two different polypeptide sequences recited in claim 1; or
a nucleic acid sequence encoding the HamA polypeptide and HamB polypeptide recited in claim 4;
(b) said Defense System Ia is encoded by
a nucleic acid sequence selected from the group referenced in Table 8 rows 2-1174 columns G, AD, and AE or the nucleic acid sequence set forth in SEQ ID NO: 14; or
a nucleic acid sequence encoding the at least two different polypeptide sequences recited in claim 1; or
a nucleic acid sequence encoding the ZorA polypeptide, the ZorB polypeptide, the ZorC polypeptide, and ZorD polypeptide recited in claim 4;
(c) said Defense System Ib is encoded by
a nucleic acid sequence selected from the group referenced in Table 8 rows 1175-1830 columns G, AD, and AE or set forth in SEQ ID NO: 15; or
a nucleic acid sequence encoding the at least two different polypeptide sequences recited in claim 1; or
a nucleic acid sequence encoding the ZorA polypeptide, the ZorB polypeptide, and ZorE polypeptide recited in claim 4;
(d) said Defense System II is encoded by
a nucleic acid sequence selected from the group referenced in Table 9 rows 2-2100 columns G, AG, and AH or set forth in the nucleic acid sequence set forth in SEQ ID NO: 10 or SEQ ID NO: 11; or
a nucleic acid sequence encoding the at least two different polypeptide sequences recited in claim 1; or
a nucleic acid sequence encoding the ThsA polypeptide and the ThsB polypeptide recited in claim 4;
(e) said Defense System IIIa is encoded by
a nucleic acid sequence selected from the group referenced in Table 10 rows 2-123 columns G, AT, and AU or set forth in SEQ ID NO: 16; or
a nucleic acid sequence encoding the at least two different polypeptide sequences recited in claim 1; or
a nucleic acid sequence encoding the DruA polypeptide, the DruB polypeptide, the DruC, the DruD, and the DruE polypeptide recited in claim 4;
(f) said Defense System IIIb is encoded by
a nucleic acid sequence selected from the group referenced in Table 10 rows 124-295 columns G, AT, AU; or
a nucleic acid sequence encoding the at least two different polypeptide sequences recited in claim 1; or
a nucleic acid sequence encoding the DruM polypeptide, the DruF polypeptide, the DruG polypeptide, and the DruE polypeptide recited in claim 4;
(g) said Defense System IIIc is encoded by
a nucleic acid sequence selected from the group referenced in Table 10 rows 296-1343 columns G, AT, and AU; or
a nucleic acid sequence encoding the at least two different polypeptide sequences recited in claim 1; or
a nucleic acid sequence encoding the DruH polypeptide and the DruE polypeptide recited in claim 4;
(h) said Defense System V is encoded by
a nucleic acid sequence selected from the group referenced in Table 13, rows 2-1247, columns H and J; or
a nucleic acid sequence encoding the SduA polypeptide of claim 1 and claim 4;
(i) said Defense System VI is encoded by
a nucleic acid sequence selected from the group referenced in Table 14, rows 2-4599, columns G, P, and Q or the sequences set forth in SEQ ID NO: 5 and SEQ ID NO: 6; or
a nucleic acid sequence encoding the at least two different polypeptide sequences recited in claim 1; or
a nucleic acid sequence encoding the GajA polypeptide and the GajB polypeptide recited in claim 4;
(j) said Defense System VII is encoded by
a nucleic acid sequence selected from the group referenced in Table 15, rows 2-2507, columns G, P, and Q or the sequences set forth in SEQ ID NO: 12 and SEQ ID NO: 13; or
a nucleic acid sequence encoding the at least two different polypeptide sequences recited in claim 1; or
a nucleic acid sequence encoding the Ptu polypeptide and the PtuB polypeptide recited in claim 4;
(k) said Defense System VIII is encoded by
a nucleic acid sequence selected from the group referenced in Table 16, rows 2-698, columns G, P, and Q or the sequences set forth in SEQ ID NO: 7 and SEQ ID NO: 8; or
a nucleic acid sequence encoding the at least two different polypeptide sequences recited in claim 1; or
a nucleic acid sequence encoding the LmuA polypeptide and LmuB polypeptide recited in claim 4;
(l) said Defense System IX is encoded by
a nucleic acid sequence selected from the group referenced in Table 17, rows 2-935, columns G, P, and Q or the sequence set forth in SEQ ID NO: 3; or
a nucleic acid sequence encoding the at least two different polypeptide sequences recited in claim 1; or
a nucleic acid sequence encoding the KwaA polypeptide and the KwaB polypeptide recited in claim 4;
(m) said Defense System Xa is encoded by
a nucleic acid sequence selected from the group referenced in Table 11, rows 2-2322, columns G, Z, and AA or the sequence set forth in SEQ ID NO: 17; or
a nucleic acid sequence encoding the at least two different polypeptide sequences recited in claim 1; or
a nucleic acid sequence encoding the JetA polypeptide, the JetB polypeptide, the JetC polypeptide, and the JetD polypeptide recited in claim 4;
(n) said Defense System Xb is encoded by
a nucleic acid sequence having at least 80% homology to a sequence selected from the group referenced in Table 11, rows 2323-2844, columns G, Z, and AA or the sequence set forth in SEQ ID NO: 18; or
a nucleic acid sequence encoding the at least two different polypeptide sequences recited in claim 1; or
a nucleic acid sequence encoding the JetAⁱⁱ polypeptide, the JetBⁱⁱ polypeptide, the JetCⁱⁱ polypeptide, and the JetDⁱⁱ polypeptide recited in claim 4;
(o) said Defense System Xc is encoded by
a nucleic acid sequence selected from the group referenced in Table 11, rows 2845-3174, columns G, Z, and AA or the sequence set forth in SEQ ID NO: 19; or
a nucleic acid sequence encoding the at least two different polypeptide sequences recited in claim 1; or
a nucleic acid sequence encoding the JetAⁱⁱⁱ polypeptide, the JetBⁱⁱⁱ polypeptide, the JetCⁱⁱⁱ polypeptide, and the JetDⁱⁱⁱ polypeptide recited in claim 4.

6. The method of claim 1, wherein
(a) for said Defense system IV, said HamA polypeptide is encoded by a nucleic acid sequence selected from the group referenced in Table 12, rows 2-1782, columns H and J, or
said HamB polypeptide is encoded by a nucleic acid sequence selected from the group referenced in Table 12, rows 2-1782, columns L and N, or
a combination thereof;
(b) for said Defense system Ia, said ZorA polypeptide is encoded by a nucleic acid sequence selected from the group referenced in Table 8 rows 2-1830 columns J and L,
said ZorB polypeptide is encoded by a nucleic acid sequence selected from the group referenced in Table 8 rows 2-1830 columns N and P,
said ZorC polypeptide is encoded by a nucleic acid sequence selected from the group referenced in Table 8 rows 2-1174 columns R and T, or
said ZorD polypeptide is encoded by a nucleic acid sequence selected from the group referenced in Table 8 rows 2-1174 columns V and X, or
a combination thereof;
(c) for said Defense system Ib, said ZorA polypeptide is encoded by a nucleic acid sequence selected from the group referenced in Table 8 rows 2-1830 columns J and L,
said ZorB polypeptide is encoded by a nucleic acid sequence selected from the group Table 8 rows 2-1830 columns N and P,
said ZorE polypeptide is encoded by a nucleic acid sequence selected from the group referenced in Table 8 rows 1175-1830 columns Z and AB, or a combination thereof;
(d) for said Defense system II, said ThsA polypeptide is encoded by a nucleic acid sequence selected from the group referenced in Table 9 rows 2-2100 columns I and K,
said ThsB polypeptide is encoded by a nucleic acid sequence selected from the group referenced in Table 9 rows 2-2100 columns M and O, Q and S, U and W, Y and AA, and AC and AE, or
a combination thereof;
(e) for said Defense system IIIa, said DruA polypeptide is encoded by a nucleic acid sequences selected from the group referenced in Table 10 rows 2-123 columns J and L, or
said DruB polypeptide is encoded by a nucleic acid sequence selected from the group referenced in Table 10 rows 2-123 columns N and P, or
said DruC polypeptide is encoded by a nucleic acid sequence selected from the group referenced in Table 10 rows 2-123 columns R and T, or
said DruD polypeptide is encoded by a nucleic acid sequence selected from the group referenced in Table 10 rows 2-123 columns V and X, or
said DruE polypeptide is encoded by a nucleic acid sequence selected from the group referenced in Table 10 rows 2-1343 columns Z and AB, or
a combination thereof;
(f) for said Defense system IIIb, said DruM polypeptide is encoded by a nucleic acid sequence selected from the group referenced in Table 10 rows 124-295 columns AD and AF, or
said DruF polypeptide is encoded by a nucleic acid sequence selected from the group referenced in Table 10 rows 124-295 columns AH and AJ, or
said DruG polypeptide is encoded by a nucleic acid sequence selected from the group referenced in Table 10 rows 124-295 columns AL and AN, or
said DruE polypeptide is encoded by a nucleic acid sequence selected from the group referenced in Table 10 rows 2-1343 columns Z and AB, or
a combination thereof;
(g) for said Defense system IIIc, said DruH polypeptide is encoded by a nucleic acid sequence selected from the group referenced in Table 10 rows 296-1343 columns AP and AR, or
said DruE polypeptide is encoded by a nucleic acid sequence selected from the group referenced in Table 10 rows 2-1343 columns Z and ZB, or
a combination thereof;
(h) for said Defense system V, said SduA polypeptide is encoded by a nucleic acid sequence selected from the group referenced in Table 13; rows 2-1247, columns H and J;
(i) for said Defense system VI, said GajA polypeptide is encoded by a nucleic acid sequence selected from the group referenced in Table 14, rows 2-4599, columns H and J, or
said GajB polypeptide is encoded by a nucleic acid sequence selected from the group referenced in Table 14, rows 2-4599, columns L and N, or
a combination thereof;
(j) for said Defense system VII, said PtuA polypeptide is encoded by a nucleic acid sequence selected from the group referenced in Table 15, rows 2-2507, columns H and J, or
said PtuB polypeptide is encoded by a nucleic acid sequence selected from the group referenced in Table 15, rows 2-2507, columns L and N, or
a combination thereof;
(k) for said Defense system VIII, said LmuA polypeptide is encoded by a nucleic acid sequence selected from the group referenced in Table 16, rows 2-698, columns H and J, or
said LmuB polypeptide is encoded by a nucleic acid sequence selected from the group referenced in Table 16, rows 2-698, columns L and N, or
a combination thereof;
(l) for said Defense system IX, said KwaA polypeptide is encoded by a nucleic acid sequence selected from the group referenced in Table 17, rows 2-935, columns H and J, or
said KwaB polypeptide is encoded by a nucleic acid sequence selected from the group referenced in Table 17, rows 2-935, columns L and N, or
a combination thereof;
(m) for said Defense system Xa, said JetA polypeptide is encoded by a nucleic acid sequence selected from the group referenced in Table 11, rows 2-2322, columns J and L, or
said JetB polypeptide is encoded by a nucleic acid sequence selected from the group referenced in Table 11, rows 2-2322, columns N and P, or
said JetC polypeptide is encoded by a nucleic acid sequence selected from the group referenced in Table 11, rows 2-2322, columns R and T, or
said JetD polypeptide is encoded by a nucleic acid sequence selected from the group referenced in Table 11, rows 2-2322, columns V and X, or
a combination thereof;
(n) for said Defense system Xb, said JetAⁱⁱ polypeptide is encoded by a nucleic acid sequence selected from the group referenced in Table 11, rows 2323-2844, columns J and L, or
said JetBⁱⁱ polypeptide is encoded by a nucleic acid sequence selected group referenced in Table 11, rows 2323-2844, columns N and P, or
said JetCⁱⁱ polypeptide is encoded by a nucleic acid sequence selected from the group referenced in Table 11, rows 2323-2844, columns R and T, or
said JetDⁱⁱ polypeptide is encoded by a nucleic acid sequence selected from the group referenced in Table 11, rows 2323-2844, columns V and X, or
a combination thereof;
(o) for said Defense system Xc, said JetAⁱⁱⁱ polypeptide is encoded by a nucleic acid sequence selected from the group referenced in Table 11, rows 2845-3174, columns J and L, or
said JetBⁱⁱⁱ polypeptide is encoded by a nucleic acid sequence selected from the group referenced in Table 11, rows 2845-3174, columns N and P, or
said JetCⁱⁱⁱ polypeptide is encoded by a nucleic acid sequence selected from the group referenced in Table 11, rows 2845-3174, columns R and T, or
said JetDⁱⁱⁱ polypeptide is encoded by a nucleic acid sequence selected from the group referenced in Table 11, rows 2845-3174, columns V and X, or
a combination thereof.

7. An at least one defense system (a)-(o), said at least one defense system comprising
(a) a Defense System IV comprising at least two different polypeptide components selected from
a HamA polypeptide comprising a pfam08878 domain or a DUF1837 domain or a combination thereof, or comprising an amino acid sequence having at least 80% identity to a sequence selected from the group referenced in Table 12 rows 2-1782 columns H and I; and
a HamB polypeptide comprising COG-1204 domain or a pfam00270 domain or a pfam00271 domain or any combination thereof, or comprising an amino acid sequence having at least 80% identity to a sequence selected from the group referenced in Table 12 rows 2-1782 columns L and M; or
(b) a Defense System Ia comprising at least two different polypeptide components selected from
a ZorA polypeptide comprising a pfam01618 domain, or comprising an amino acid sequence having at least 80% identity to a sequence selected from the group referenced in Table 8 rows 2-1830 columns J and K;
a ZorB polypeptide comprising a pfam13677 domain or a pfam00691 domain or a combination thereof, or comprising an amino acid sequence having at least 80% identity to a sequence selected from the group referenced in Table 8 rows 2-1830 columns N and O;
a ZorC polypeptide comprising a pfam15611 domain, or comprising an amino acid sequence having at least 80% identity to a sequence selected from the group referenced in Table 8 rows 2-1174 columns R and S; and
a ZorD polypeptide comprising a pfam00176 domain or a pfam00271 domain or a combination thereof, or comprising an amino acid sequence having at least 80% identity to a sequence selected from the group referenced in Table 8 rows 2-1174 columns V and W;
or
(c) a Defense System Ib comprising a ZorE polypeptide comprising a pfam01844 domain or a COG3183 domain or a combination thereof, or comprising an amino acid sequence having at least 80% identity to a sequence selected from the group referenced in Table 8 rows 1175-1830 columns Z and AA; and at least one other polypeptide component selected from
a ZorA polypeptide comprising a pfam01618 domain, or comprising an amino acid sequence having at least 80% identity to a sequence selected from the group referenced in Table 8 rows 2-1830 columns J and K; and
a ZorB polypeptide comprising a pfam13677 domain or a pfam00691 domain or a combination thereof, or comprising an amino acid sequence having at least 80% identity to a sequence selected from the group referenced in Table 8 rows 2-1830 columns N and O; or
(d) a Defense System II comprising at least two different polypeptide components selected from
a ThsA polypeptide comprising a pfam13289 domain or a pfam14519 domain or a combination thereof, or comprising an amino acid sequence having at least 80% identity to a sequence selected from the group referenced in Table 9 rows 2-2100 columns I and J; and
a ThsB polypeptide comprising a pfam13676 domain or a pfam08937 domain or a pfam08357 domain or any combination thereof, or comprising an amino acid sequence having at least 80% identity to a sequence selected from the group referenced in Table 9 rows 2-2100 columns M and N, Q and R, U and V, Y and Z, and AC and AD; or
(e) a Defense System IIIa comprising at least two different polypeptide components selected from
a DruA polypeptide comprising a DUF4338 domain or a pfam14236 domain or a combination thereof, or comprising an amino acid sequence having at least 80% identity to a sequence selected from the group referenced in Table 10 rows 2-123 columns J and K, or a combination thereof;
a DruB polypeptide comprising an amino acid sequence having at least 80% identity to a sequence selected from the group referenced in Table 10 rows 2-123 columns N and O;
a DruC polypeptide comprising an amino acid sequence having at least 80% identity to a sequence selected from the group referenced in Table 10 rows 2-123 columns R and S;
a DruD polypeptide comprising an amino acid sequence having at least 80% identity to a sequence selected from the group referenced in Table 10 rows 2-123 columns V and W; and
a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA; or
(f) a Defense System IIIb comprising at least two different polypeptide components selected from
a DruM polypeptide comprising a pfam00145 domain or a COG0270 domain or a combination thereof, or comprising an amino acid sequence having at least 80% identity to a sequence selected from the group referenced in Table 10 rows 124-295 columns AD and AE;
a DruF polypeptide comprising an amino acid sequence having at least 80% identity to a sequence selected from the group referenced in Table 10 rows 124-295 columns AH and AI;
a DruG polypeptide comprising an amino acid sequence having at least 80% identity to a sequence selected from the group referenced in Table 10 rows 124-295 columns AL and AM; and
a DruE polypeptide comprising a pfam00270 domain or a pfam00271 domain or a pfam09369 domain or a DUF1998 domain, or any combination thereof, or comprising an amino acid sequence having at least 80% identity to a sequence selected from the group referenced in Table 10 rows 2-1343 columns Z and AA; or
(g) a Defense System IIIc comprising at least two different polypeptide components selected from
a DruH polypeptide comprising an amino acid sequence having at least 80% identity to a sequence selected from the group referenced in Table 10 rows 296-1343 columns AP and AQ; and
a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA; or
(h) a Defense System V comprising a SduA polypeptide comprising a pfam14082 domain or a pfam01939 domain or a combination thereof, or comprising an amino acid sequence having at least 80% identity to a sequence selected from the group referenced in Table 13 rows 2-1247 columns H and I; or
(i) a Defense System VI comprising at least two different polypeptide components selected from
a GajA polypeptide comprising a pfam13175 domain or a COG3593 domain or a combination thereof, or comprising an amino acid sequence having at least 80% identity to a sequence selected from the group referenced in Table 14 rows 2-4599 columns H and I; and
a GajB polypeptide comprising a pfam04257 domain or a pfam00580 domain or a pfam13361 domain or any combination thereof, or comprising an amino acid sequence having at least 80% identity to a sequence selected from the group referenced in Table 14 rows 2-4599 columns L and M; or
(j) a Defense System VII comprising at least two different polypeptide components selected from
a PtuA polypeptide comprising a pfam13304 domain or a pfam02463 domain or a combination thereof, or comprising an amino acid sequence having at least 80% identity to a sequence selected from the group referenced in Table 15 rows 2-12507 columns H and I; and
a PtuB polypeptide comprising pfam13395 domain or a pfam 01844 domain or a combination thereof, or comprising an amino acid sequence having at least 80% identity to a sequence selected from the group referenced in Table 15 rows 2-2507 columns L and M; or
(k) a Defense System VIII comprising at least two different polypeptide components selected from
a LmuA polypeptide comprising a pfam14130 domain or a DUF4297 domain or a combination thereof, or comprising an amino acid sequence having at least 80% identity to a sequence selected from the group referenced in Table 16 rows 2-698 columns H and I; and
a LmuB polypeptide comprising a pfam02463 domain, or comprising an amino acid sequence having at least 80% identity to a sequence selected from the group referenced in Table 16 rows 2-698 columns L and M; or
(l) a Defense System IX comprising at least two different polypeptide components selected from
a KwaA polypeptide comprising an amino acid sequence having at least 80% identity to a sequence selected from the group referenced in Table 17 rows 2-935 columns H and I; and
a KwaB polypeptide comprising a pfam16162 domain or a DUF4868 domain or a combination thereof, or comprising an amino acid sequence having at least 80% identity to a sequence selected from the group referenced in Table 17 rows 2-935 columns L and M; or
(m) a Defense System Xa comprising at least two different polypeptide components selected from
a JetA polypeptide comprising a pfam11855 domain or a DUF3375 domain or a combination thereof, or comprising an amino acid sequence having at least 80% identity to a sequence selected from the group referenced in Table 11 rows 2-2322 columns J and K;
a JetB polypeptide comprising a pfam13835 domain or a DUF4194 domain or a combination thereof, or comprising an amino acid sequence having at least 80% identity to a sequence selected from the group referenced in Table 11 rows 2-2322 columns N and O;
a JetC polypeptide comprising a pfam13555 domain or a pfam13558 domain or a COG4913 domain or any combination thereof, or comprising an amino acid sequence having at least 80% identity to a sequence selected from the group referenced in Table 11 rows 2-2322 columns R and S; and
a JetD polypeptide comprising a pfam11795 domain or a DUF3322 domain or a pfam09983 domain or a DUF2220 domain or a COG4924 domain or any combination thereof, or comprising an amino acid sequence having at least 80% identity to a sequence selected from the group referenced in Table 11 rows 2-2322 columns V and W; or
(n) a Defense System Xb comprising at least two different polypeptide components selected from
a JetAⁱⁱ polypeptide comprising a pfam09660 domain or a DUF2397 domain or a combination thereof, or comprising an amino acid sequence having at least 80% identity to a sequence selected from the group referenced in Table 11 rows 2323-2844 columns J and K;
a JetBⁱⁱ polypeptide comprising a pfam09661 domain or a DUF2398 domain or a combination thereof, or comprising an amino acid sequence having at least 80% identity to a sequence selected from the group referenced in Table 11 rows 2323-2844 columns N and O;
a JetCⁱⁱ polypeptide comprising a pfam13558 domain comprising an amino acid sequence having at least 80% identity to a sequence selected from the group referenced in Table 11 rows 2323-2844 columns R and S; and
a JetDⁱⁱ polypeptide comprising a pfam11796 domain or a DUF3323 domain or a pfam09664 domain or a DUF2399 domain or a combination thereof, or comprising an amino acid sequence having at least 80% identity to a sequence selected from the group referenced in Table 11 rows 2323-2844 columns V and W; or
(o) a Defense System Xc comprising at least two different polypeptide components selected from
a JetAⁱⁱⁱ polypeptide comprising an amino acid sequence having at least 80% identity to a sequence selected from the group referenced in Table 11 rows 2845-3174 columns J and K;
a JetBⁱⁱⁱ polypeptide comprising an amino acid sequence having at least 80% identity to a sequence selected from the group referenced in Table 11 rows 2845-3174 columns N and O;
a JetCⁱⁱⁱ polypeptide comprising a COG1196 domain or comprising an amino acid sequence having at least 80% identity to a sequence selected from the group referenced in Table 11 rows 2845-3174 columns R and S; and
a JetDⁱⁱⁱ polypeptide comprising a pfam09983 domain or a pfam09664 domain or a DUF2220 or a DUF2399 domain or any combination thereof, or comprising an amino acid sequence having at least 80% identity to a sequence selected from the group referenced in Table 11 rows 2845-3174 columns V and W; or
(p) any combination of the Defense Systems Ia-Xc (a)-(o);
wherein said at least one defense system comprises a non-naturally occurring combination of components.

8. The defense system of claim 7, wherein
(a) said Defense System IV comprises the HamA polypeptide and the HamB polypeptide; or
(b) said Defense System Ia comprises the ZorA polypeptide, the ZorB polypeptide, the ZorC polypeptide, and the ZorD polypeptide; or
(c) said Defense System Ib comprises the ZorA polypeptide, the ZorB polypeptide, and the ZorE polypeptide; or
(d) said Defense System II comprises the ThsA polypeptide, and the ThsB polypeptide; or
(e) said Defense System IIIa comprises the DruA polypeptide, the DruB polypeptide, the DruC polypeptide, the DruD polypeptide, and the DruE polypeptide; or
(f) said Defense System IIIb comprises the DruM polypeptide, the DruF polypeptide, the DruG polypeptide, and the DruE polypeptide; or
(f) said Defense System IIIc comprises the DruH polypeptide and the DruE polypeptide; or
(h) said Defense System V comprises the SduA polypeptide; or
(i) said Defense System VI comprises the GajA polypeptide and the GajB polypeptide; or
(j) said Defense System VII comprises the PtuA polypeptide and the PtuB polypeptide; or
(k) said Defense System VIII comprises the LmuA polypeptide and the LmuB polypeptide; or
(l) said Defense System IX comprises the KwaA polypeptide and the KwaB polypeptide; or
(m) said Defense System Xa comprises the JetA polypeptide, the JetB polypeptide, the JetC polypeptide, and the JetD polypeptide; or
(n) said Defense System Xb comprises the JetAⁱⁱ polypeptide, the JetBⁱⁱ polypeptide, the JetCⁱⁱ polypeptide, and the JetDⁱⁱ polypeptide; or
(o) said Defense System Xc comprises the JetAⁱⁱⁱ polypeptide, the JetBⁱⁱⁱ polypeptide, the JetCⁱⁱⁱ polypeptide, and the JetDⁱⁱ polypeptide.

9. The at least one defense system of claim 7, wherein said defense system provides a host cell with resistance to foreign nucleic acid invasion.

10. The at least one defense system of claim 9, wherein said resistance to foreign nucleic acid invasion comprises resistance to at least one phage infection, or resistance to plasmid transformation, or resistance to entry of a conjugative element, or any combination thereof.

11. A nucleic acid construct encoding an at least one defense system (a)-(o), said nucleic acid construct comprising
(a) a nucleic acid construct encoding a Defense System IV comprising at least two different polypeptide components selected from
a HamA polypeptide comprising a pfam08878 domain or a DUF1837 domain or a combination thereof, or comprising an amino acid sequence having at least 80% identity to a sequence selected from the group referenced in Table 12 rows 2-1782 columns H and I; and
a HamB polypeptide comprising COG1204 domain or a pfam00270 domain or a pfam00271 domain or any combination thereof, or comprising an amino acid sequence having at least 80% identity to a sequence selected from the group referenced in Table 12 rows 2-1782 columns L and M; or
(b) a nucleic acid construct encoding a Defense System Ia comprising at least two different polypeptide components selected from
a ZorA polypeptide comprising a pfam01618 domain, or comprising an amino acid sequence having at least 80% identity to a sequence selected from the group referenced in Table 8 rows 2-1830 columns J and K;
a ZorB polypeptide comprising a pfam 13677 domain or a pfam00691 domain or a combination thereof, or comprising an amino acid sequence having at least 80% identity to a sequence selected from the group referenced in Table 8 rows 2-1830 columns N and O;
a ZorC polypeptide comprising a pfam15611 domain, or comprising an amino acid sequence having at least 80% identity to a sequence selected from the group referenced in Table 8 rows 2-1174 columns R and S; and
a ZorD polypeptide comprising a pfam00176 domain or a pfam00271 domain or a combination thereof, or comprising an amino acid sequence having at least 80% identity to a sequence selected from the group referenced in Table 8 rows 2-1174 columns V and W;
or
(c) a nucleic acid construct encoding a Defense System Ib comprising a ZorE polypeptide comprising a pfam01844 domain or a COG3183 domain or a combination thereof, or comprising an amino acid sequence having at least 80% identity to a sequence selected from the group referenced in Table 8 rows 1175-1830 columns Z and AA; and at least one other polypeptide component selected from
a ZorA polypeptide comprising a pfam01618 domain, or comprising an amino acid sequence having at least 80% identity to a sequence selected from the group referenced in Table 8 rows 2-1830 columns J and K; and
a ZorB polypeptide comprising a pfam13677 domain or a pfam00691 domain or a combination thereof, or comprising an amino acid sequence having at least 80% identity to a sequence selected from the group referenced in Table 8 rows 2-1830 columns N and O; or
(d) a nucleic acid construct encoding a Defense System II comprising at least two different polypeptide components selected from
a ThsA polypeptide comprising a pfam13289 domain or a pfam14519 domain or a combination thereof, or comprising an amino acid sequence having at least 80% identity to a sequence selected from the group referenced in Table 9 rows 2-2100 columns I and J; and
a ThsB polypeptide comprising a pfam13676 domain or a pfam08937 domain or a pfam08357 domain or any combination thereof, or comprising an amino acid sequence having at least 80% identity to a sequence selected from the group referenced in Table 9 rows 2-2100 columns M and N, Q and R, U and V, Y and Z, and AC and AD; or
(e) a nucleic acid construct encoding a Defense System IIIa comprising at least two different polypeptide components selected from
a DruA polypeptide comprising a DUF4338 domain or a pfam14236 domain or a combination thereof, or comprising an amino acid sequence having at least 80% identity to a sequence selected from the group referenced in Table 10 rows 2-123 columns J and K, or a combination thereof;
a DruB polypeptide comprising an amino acid sequence having at least 80% identity to a sequence selected from the group referenced in Table 10 rows 2-123 columns N and O;
a DruC polypeptide comprising an amino acid sequence having at least 80% identity to a sequence selected from the group referenced in Table 10 rows 2-123 columns R and S;
a DruD polypeptide comprising an amino acid sequence having at least 80% identity to a sequence selected from the group referenced in Table 10 rows 2-123 columns V and W; and
a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA; or
(f) a nucleic acid construct encoding a Defense System IIIb comprising at least two different polypeptide components selected from
a DruM polypeptide comprising a pfam00145 domain or a COG0270 domain or a combination thereof, or comprising an amino acid sequence having at least 80% identity to a sequence selected from the group referenced in Table 10 rows 124-295 columns AD and AE;
a DruF polypeptide comprising an amino acid sequence having at least 80% identity to a sequence selected from the group referenced in Table 10 rows 124-295 columns AH and AI;
a DruG polypeptide comprising an amino acid sequence having at least 80% identity to a sequence selected from the group referenced in Table 10 rows 124-295 columns AL and AM; and
a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA; or
(g) a nucleic acid construct encoding a Defense System IIIc comprising at least two different polypeptide components selected from
a DruH polypeptide comprising an amino acid sequence having at least 80% identity to a sequence selected from the group referenced in Table 10 rows 296-1343 columns AP and AQ; and
a DruE polypeptide comprising a pfam00270 domain or a pfam00271 domain or a pfam09369 domain or a DUF1998 domain or any combination thereof, or comprising an amino acid sequence having at least 80% identity to a sequence selected from the group referenced in Table 10 rows 2-1343 columns Z and AA; or
(h) a nucleic acid construct encoding a Defense System V comprising a SduA polypeptide comprising a pfam14082 domain or a pfam01939 domain or a combination thereof, or comprising an amino acid sequence having at least 80% identity to a sequence selected from the group referenced in Table 13 rows 2-1247 columns H and I; or
(i) a nucleic acid construct encoding a Defense System VI comprising at least two different polypeptide components selected from
a GajA polypeptide comprising a pfam13175 domain or a COG3593 domain or a combination thereof, or comprising an amino acid sequence having at least 80% identity to a sequence selected from the group referenced in Table 14 rows 2-4599 columns H and I; and
a GajB polypeptide comprising a pfam04257 domain or a pfam00580 domain or a pfam13361 domain or any combination thereof, or comprising an amino acid sequence having at least 80% identity to a sequence selected from the group referenced in Table 14 rows 2-4599 columns L and M; or
(j) a nucleic acid construct encoding a Defense System VII comprising at least two different polypeptide components selected from
a PtuA polypeptide comprising a pfam13304 domain or a pfam02463 domain or a combination thereof, or comprising an amino acid sequence having at least 80% identity to a sequence selected from the group referenced in Table 15 rows 2-12507 columns H and I; and
a PtuB polypeptide comprising pfam13395 domain or a pfam 01844 domain or a combination thereof, or comprising an amino acid sequence having at least 80% identity to a sequence selected from the group referenced in Table 15 rows 2-2507 columns L and M; or
(k) a nucleic acid construct encoding a Defense System VIII comprising at least two different polypeptide components selected from
a LmuA polypeptide comprising a pfam14130 domain or a DUF4297 domain or a combination thereof, or comprising an amino acid sequence having at least 80% identity to a sequence selected from the group referenced in Table 16 rows 2-698 columns H and I; and
a LmuB polypeptide comprising a pfam02463 domain, or comprising an amino acid sequence having at least 80% identity to a sequence selected from the group referenced in Table 16 rows 2-698 columns L and M; or
(l) a nucleic acid construct encoding a Defense System IX comprising at least different polypeptide components selected from
a KwaA polypeptide comprising an amino acid sequence having at least 80% identity to a sequence selected from the group referenced in Table 17 rows 2-935 columns II and I; and
a KwaB polypeptide comprising a pfam16162 domain or a DUF4868 domain or a combination thereof, or comprising an amino acid sequence having at least 80% identity to a sequence selected from the group referenced in Table 17 rows 2-935 columns L and M; or
(m) a nucleic acid construct encoding a Defense System Xa comprising at least two different polypeptide components selected from
a JetA polypeptide comprising a pfam11855 domain or a DUF3375 domain or a combination thereof, or comprising an amino acid sequence having at least 80% identity to a sequence selected from the group referenced in Table 11 rows 2-2322 columns J and K;
a JetB polypeptide comprising a pfam13835 domain or a DUF4194 domain or a combination thereof, or comprising an amino acid sequence having at least 80% identity to a sequence selected from the group referenced in Table 11 rows 2-2322 columns N and O;
a JetC polypeptide comprising a pfam13555 domain or a pfam13558 domain or a COG4913 domain or any combination thereof, or comprising an amino acid sequence having at least 80% identity to a sequence selected from the group referenced in Table 11 rows 2-2322 columns R and S; and
a JetD polypeptide comprising a pfam11795 domain or a DUF3322 domain or a pfam09983 domain or a DUF2220 domain or a COG4924 domain or any combination thereof, or comprising an amino acid sequence having at least 80% identity to a sequence selected from the group referenced in Table 11 rows 2-2322 columns V and W; or
(n) a nucleic acid construct encoding a Defense System Xb comprising at least two different polypeptide components selected from
a JetAⁱⁱ polypeptide comprising a pfam09660 domain or a DUF2397 domain or a combination thereof, or comprising an amino acid sequence having at least 80% identity to a sequence selected from the group referenced in Table 11 rows 2323-2844 columns J and K;
a JetBⁱⁱ polypeptide comprising a pfam09661 domain or a DUF2398 domain or a combination thereof, or comprising an amino acid sequence having at least 80% identity to a sequence selected from the group referenced in Table 11 rows 2323-2844 columns N and O;
a JetCⁱⁱ polypeptide comprising a pfam13558 domain comprising an amino acid sequence having at least 80% identity to a sequence selected from the group referenced in Table 11 rows 2323-2844 columns R and S; and
a JetDⁱⁱ polypeptide comprising a pfam11796 domain or a DUF3323 domain or a pfam09664 domain or a DUF2399 domain or a combination thereof, or comprising an amino acid sequence having at least 80% identity to a sequence selected from the group referenced in Table 11 rows 2323-2844 columns V and W; or
(o) a nucleic acid construct encoding a Defense System Xc comprising at least two different polypeptide components selected from
a JetAⁱⁱⁱ polypeptide comprising an amino acid sequence having at least 80% identity to a sequence selected from the group referenced in Table 11 rows 2845-3174 columns J and K;
a JetBⁱⁱⁱ polypeptide comprising an amino acid sequence having at least 80% identity to a sequence selected from the group referenced in Table 11 rows 2845-3174 columns N and O;
a JetCⁱⁱⁱ polypeptide comprising a COG1196 domain or comprising an amino acid sequence having at least 80% identity to a sequence selected from the group referenced in Table 11 rows 2845-3174 columns R and S; and
a JetDⁱⁱⁱ polypeptide comprising a pfam09983 domain or a pfam09664 domain or a DUF2220 or a DUF2399 domain or any combination thereof, or comprising an amino acid sequence having at least 80% identity to a sequence selected from the group referenced in Table 11 rows 2845-3174 columns V and W; or
(p) any combination of nucleic acid constructs encoding a Defense Systems Ia-Xc (a)-(o);
wherein each nucleic acid construct of each Defense System (a)-(o) further comprises a regulatory element operably linked to said construct comprising a cis-acting regulatory element for directing expression of said nucleic acid sequence, or a transmissible element for directing transfer of said nucleic acid sequence from one cell to another, or a recombination element for integrating said nucleic acid sequence into a genome of a cell transfected with said construct, or an element providing episomal maintenance of said construct within a cell transfected with said construct, or any combination thereof.

12. The nucleic acid construct of claim 11, wherein said expressed defense system comprises
(a) a Defense System IV comprising the HamA polypeptide, and the HamB polypeptide; or
(b) a Defense System Ia comprising the ZorA polypeptide, the ZorB polypeptide, the ZorC polypeptide, and the ZorD polypeptide; or
(c) a Defense System Ib comprising the ZorA polypeptide, the ZorB polypeptide, and the ZorE polypeptide; or
(d) a Defense System II comprising the ThsA polypeptide, and the ThsB polypeptide; or
(e) a Defense System IIIa comprising the DruA polypeptide, the DruB polypeptide, the DruC polypeptide, the DruD polypeptide, and the DruE polypeptide; or
(f) a Defense System IIIb comprising the Drug polypeptide, the DruF polypeptide, the DruG polypeptide, and the DruE polypeptide; or
(g) a Defense System IIIc comprising the DruH polypeptide and the DruE polypeptide; or
(h) a Defense System V comprising the SduA polypeptide; or
(i) a Defense System VI comprising the GajA polypeptide and the GajB polypeptide; or
(j) a Defense System VII comprising the PtuA polypeptide and the PtuB polypeptide; or
(k) a Defense System VIII comprising the LmuA polypeptide and the LmuB polypeptide; or
(l) a Defense System IX comprising the KwaA polypeptide and the KwaB polypeptide; or
(m) a Defense System Xa comprising the JetA polypeptide, the JetB polypeptide, the JetC polypeptide, and the JetD polypeptide; or
(n) a Defense System Xb comprising the JetAⁱⁱ polypeptide, the JetBⁱⁱ polypeptide, the JetCⁱⁱ polypeptide, and the JetDⁱⁱ polypeptide; or
(o) a Defense System Xc comprising the JetAⁱⁱⁱ polypeptide, the JetBⁱⁱⁱ polypeptide, the JetCⁱⁱⁱ polypeptide, and the JetDⁱⁱⁱ polypeptide.

13. The nucleic acid construct of claim 11, wherein
(a) said Defense System IV is encoded by
a nucleic acid sequence selected from the group referenced in Table 12, rows 2-1782, columns G, P, and Q or the nucleic acid sequence set forth in SEQ ID NO: 4; or
a nucleic acid sequence encoding the at least two different polypeptide sequences recited in claim 1; or
a nucleic acid sequence encoding the HamA polypeptide and HamB polypeptide recited in claim 4;
(b) said Defense System Ia is encoded by,
a nucleic acid sequence selected from the group referenced in Table 8 rows 2-1174 columns G, AD, and AE or the nucleic acid sequence set forth in SEQ ID NO: 14; or
a nucleic acid sequence encoding the at least two different polypeptide sequences recited in claim 1; or
a nucleic acid sequence encoding the ZorA polypeptide, the ZorB polypeptide, the ZorC polypeptide, and ZorD polypeptide recited in claim 4;
(c) said Defense System Ib is encoded by
a nucleic acid sequence selected from the group referenced in Table 8 rows 1175-1830 columns G, AD, and AE or set forth in SEQ ID NO: 15; or
a nucleic acid sequence encoding the at least two different polypeptide sequences recited in claim 1; or
a nucleic acid sequence encoding the ZorA polypeptide, the ZorB polypeptide, and ZorE polypeptide recited in claim 4;
(d) said Defense System II is encoded by
a nucleic acid sequence selected from the group referenced in Table 9 rows 2-2100 columns G, AG, and AH or set forth in the nucleic acid sequence set forth in SEQ ID NO: 10 or SEQ ID NO: 11; or
a nucleic acid sequence encoding the at least two different polypeptide sequences recited in claim 1; or
a nucleic acid sequence encoding the ThsA polypeptide and the ThsB polypeptide recited in claim 4;
(e) said Defense System IIIa is encoded by
a nucleic acid sequence selected from the group referenced in Table 10 rows 2-123 columns G, AT, and AU or set forth in SEQ ID NO: 16; or
a nucleic acid sequence encoding the at least two different polypeptide sequences recited in claim 1; or
a nucleic acid sequence encoding the DruA polypeptide, the DruB polypeptide, the DruC, the DruD, and the DruE polypeptide recited in claim 4;
(f) said Defense System IIIb is encoded by
a nucleic acid sequence selected from the group referenced in Table 10 rows 124-295 columns G, AT, AU; or
a nucleic acid sequence encoding the at least two different polypeptide sequences recited in claim 1; or
a nucleic acid sequence encoding the DruM polypeptide, the DruF polypeptide, the DruG polypeptide, and the DruE polypeptide recited in claim 4;
(g) said Defense System IIIc is encoded by
a nucleic acid sequence selected from the group referenced in Table 10 rows 296-1343 columns G, AT, and AU; or
a nucleic acid sequence encoding the at least two different polypeptide sequences recited in claim 1; or
a nucleic acid sequence encoding the DruH polypeptide and the DruE polypeptide recited in claim 4;
(h) said Defense System V is encoded by
a nucleic acid sequence selected from the group referenced in Table 13; rows 2-1247, columns H and J; or
a nucleic acid sequence encoding the SduA polypeptide of claim 1 and claim 4;
(i) said Defense System VI is encoded by
a nucleic acid sequence selected from the group referenced in Table 14, rows 2-4599, columns G, P, and Q or the sequences set forth in SEQ ID NO: 5 and SEQ ID NO: 6; or
a nucleic acid sequence encoding the at least two different polypeptide sequences recited in claim 1; or
a nucleic acid sequence encoding the GajA polypeptide and the GajB polypeptide recited in claim 4;
(j) said Defense System VII is encoded by
a nucleic acid sequence selected from the group referenced in Table 15, rows 2-2507, columns G, P, and Q or the sequences set forth in SEQ ID NO: 12 and SEQ ID NO: 13; or
a nucleic acid sequence encoding the at least two different polypeptide sequences recited in claim 1; or
a nucleic acid sequence encoding the Ptu polypeptide and the PtuB polypeptide recited in claim 4;
(k) said Defense System VIII is encoded by
a nucleic acid sequence selected from the group referenced in Table 16, rows 2-698, columns G, P, and Q or the sequences set forth in SEQ ID NO: 7 and SEQ ID NO: 8; or
a nucleic acid sequence encoding the at least two different polypeptide sequences recited in claim 1; or
a nucleic acid sequence encoding the LmuA polypeptide and LmuB polypeptide recited in claim 4;
(l) said Defense System IX is encoded by
a nucleic acid sequence selected from the group referenced in Table 17, rows 2-935, columns G, P, and Q or the sequence set forth in SEQ ID NO: 3; or
a nucleic acid sequence encoding the at least two different polypeptide sequences recited in claim 1; or
a nucleic acid sequence encoding the KwaA polypeptide and the KwaB polypeptide recited in claim 4;
(m) said Defense System Xa is encoded by
a nucleic acid sequence selected from the group referenced in Table 11, rows 2-2322, columns G, Z, and AA or the sequence set forth in SEQ ID NO: 17; or
a nucleic acid sequence encoding the at least two different polypeptide sequences recited in claim 1; or
a nucleic acid sequence encoding the JetA polypeptide, the JetB polypeptide, the JetC polypeptide, and the JetD polypeptide recited in claim 4;
(n) said Defense System Xb is encoded by
a nucleic acid sequence having at least 80% homology to a sequence selected from the group referenced in Table 11, rows 2323-2844, columns G, Z, and AA or the sequence set forth in SEQ ID NO: 18; or
a nucleic acid sequence encoding the at least two different polypeptide sequences recited in claim 1; or
a nucleic acid sequence encoding the JetAⁱⁱ polypeptide, the JetBⁱⁱ polypeptide, the JetCⁱⁱ polypeptide, and the JetDⁱⁱ polypeptide recited in claim 4;
(o) said Defense System Xc is encoded by
a nucleic acid sequence selected from the group referenced in Table 11, rows 2845-3174, columns G, Z, and AA or the sequence set forth in SEQ ID NO: 19; or
a nucleic acid sequence encoding the at least two different polypeptide sequences recited in claim 1; or
a nucleic acid sequence encoding the JetAⁱⁱⁱ polypeptide, the JetBⁱⁱⁱ polypeptide, the JetCⁱⁱⁱ polypeptide, and the JetDⁱⁱⁱ polypeptide recited in claim 4.

14. The nucleic acid construct of claim 11, wherein
(a) said HamA polypeptide is encoded by a nucleic acid sequence selected from the group referenced in Table 12, rows 2-1782, columns H and J, or
said HamB polypeptide is encoded by a nucleic acid sequence selected from the group referenced in Table 12, rows 2-1782, columns L and N, or a combination thereof;
(b) said ZorA polypeptide is encoded by a nucleic acid sequence selected from the group referenced in Table 8 rows 2-1830 columns J and L,
said ZorB polypeptide is encoded by a nucleic acid sequence selected from the group referenced in Table 8 rows 2-1830 columns N and P,
said ZorC polypeptide is encoded by a nucleic acid sequence selected from the group referenced in Table 8 rows 2-1174 columns R and T, or
said ZorD polypeptide is encoded by a nucleic acid sequence selected from the group referenced in Table 8 rows 2-1174 columns V and X, or
a combination thereof;
(c) said ZorA polypeptide is encoded by a nucleic acid sequence selected from the group referenced in Table 8 rows 2-1830 columns J and L,
said ZorB polypeptide is encoded by a nucleic acid sequence selected from the group Table 8 rows 2-1830 columns N and P,
said ZorE polypeptide is encoded by a nucleic acid sequence selected from the group referenced in Table 8 rows 1175-1830 columns Z and AB, or a combination thereof;
(d) said ThsA polypeptide is encoded by a nucleic acid sequence selected from the group referenced in Table 9 rows 2-2100 columns I and K,
said ThsB polypeptide is encoded by a nucleic acid sequence selected from the group referenced in Table 9 rows 2-2100 columns M and O, Q and S, U and W, Y and AA, and AC and AE, or
a combination thereof;
(e) said DruA polypeptide is encoded by a nucleic acid sequence selected from the group referenced in Table 10 rows 2-123 columns J and L, or
said DruB polypeptide is encoded by a nucleic acid sequence selected from the group referenced in Table 10 rows 2-123 columns N and P, or
said DruC polypeptide is encoded by a nucleic acid sequence selected from the group referenced in Table 10 rows 2-123 columns R and T, or
said DruD polypeptide is encoded by a nucleic acid sequence selected from the group referenced in Table 10 rows 2-123 columns V and X, or
said DruE polypeptide is encoded by a nucleic acid sequence selected from the group referenced in Table 10 rows 2-1343 columns Z and AB, or a combination thereof;
(f) said DruM polypeptide is encoded by a nucleic acid sequence selected from the group referenced in Table 10 rows 124-295 columns AD and AF, or
said DruF polypeptide is encoded by a nucleic acid sequence selected from the group referenced in Table 10 rows 124-295 columns AH and AJ, or
said DruG polypeptide is encoded by a nucleic acid sequence selected from the group referenced in Table 10 rows 124-295 columns AL and AN, or
said DruE polypeptide is encoded by a nucleic acid sequence selected from the group referenced in Table 10 rows 2-1343 columns Z and AB, or
a combination thereof;
(g) said DruH polypeptide is encoded by a nucleic acid sequence selected from the group referenced in Table 10 rows 296-1343 columns AP and AR, or
said DruE polypeptide is encoded by a nucleic acid sequence selected from the group referenced in Table 10 rows 2-1343 columns Z and AB, or
a combination thereof;
(h) said SduA polypeptide is encoded by a nucleic acid sequence selected from the group referenced in Table 13; rows 2-1247, columns H and J;
(i) said GajA polypeptide is encoded by a nucleic acid sequence selected from the group referenced in Table 14, rows 2-4599, columns H and J, or
said GajB polypeptide is encoded by a nucleic acid sequence selected from the group referenced in Table 14, rows 2-4599, columns L and N; or
a combination thereof;
(j) said PtuA polypeptide is encoded by a nucleic acid sequence selected from the group referenced in Table 15, rows 2-2507, columns H and J, or
said PtuB polypeptide is encoded by a nucleic acid sequence selected from the group referenced in Table 15, rows 2-2507, columns L and N, or
a combination thereof;
(k) said LmuA polypeptide is encoded by a nucleic acid sequence selected from the group referenced in Table 16, rows 2-698, columns H and J, or
said LmuB polypeptide is encoded by a nucleic acid sequence selected from the group referenced in Table 16, rows 2-698, columns L and N, or
a combination thereof;
(l) said KwaA polypeptide is encoded by a nucleic acid sequence selected from the group referenced in Table 17, rows 2-935, columns H and J, or
said KwaB polypeptide is encoded by a nucleic acid sequence selected from the group referenced in Table 17, rows 2-935, columns L and N, or
a combination thereof;
(m) said JetA polypeptide is encoded by a nucleic acid sequence selected from the group referenced in Table 11, rows 2-2322, columns J and L, or
said JetB polypeptide is encoded by a nucleic acid sequence selected from the group referenced in Table 11, rows 2-2322; columns N and P, or
said JetC polypeptide is encoded by a nucleic acid sequence selected from the group referenced in Table 11, rows 2-2322, columns R and T, or
said JetD polypeptide is encoded by a nucleic acid sequence selected from the group referenced in Table 11, rows 2-2322, columns V and X, or
a combination thereof;
(n) said JetAⁱⁱ polypeptide is encoded by a nucleic acid sequence selected from the group referenced in Table 11, rows 2323-2844, columns J and L, or
said JetBⁱⁱ polypeptide is encoded by a nucleic acid sequence selected from the group referenced in Table 11, rows 2323-2844, columns N and P, or
said JetCⁱⁱ polypeptide is encoded by a nucleic acid sequence selected from the group referenced in Table 11, rows 2323-2844, columns R and T, or
said JetDⁱⁱ polypeptide is encoded by a nucleic acid sequence selected from the group referenced in Table 11, rows 2323-2844, columns V and X, or
a combination thereof;
(o) said JetAⁱⁱⁱ polypeptide is encoded by a nucleic acid sequence selected from the group referenced in Table 11, rows 2845-3174, column J and L, or
said JetBⁱⁱⁱ polypeptide is encoded by a nucleic acid sequence selected from the group referenced in Table 11, rows 2845-3174, columns N and O, or
said JetCⁱⁱⁱ polypeptide is encoded by a nucleic acid sequence selected from the group referenced in Table 11, rows 2845-3174, columns R and T, or
said JetDⁱⁱⁱ polypeptide is encoded by a nucleic acid sequence selected from the group referenced in Table 11, rows 2845-3174, columns V and X, or
a combination thereof.

15. The nucleic acid construct of claim 11, wherein expression of said defense system in a host cells provides the host cell with resistance to foreign nucleic acid invasion.

16. The nucleic acid construct of claim 15, wherein said resistance to foreign nucleic acid invasion comprises resistance to at least one phage infection, or resistance to plasmid transformation, or resistance to entry of a conjugation element, or any combination thereof

17. A transmissible genetic element or an expression vector comprising the nucleic acid construct of claim 1.

18. An isolated cell expressing a nucleic acid construct of claim 1.

19. The isolated cell of claim 18, wherein said cell comprises a gram-positive bacterium or a gram-negative bacterium.

20. The isolated cell of claim 18, wherein said cell comprises resistance to foreign nucleic acid invasion.

21. The isolated cell of claim 20, wherein resistance to foreign nucleic acid invasion comprises resistance to at least one phage infection, or reduced plasmid transformation efficiency, or resistance to entry of a conjugation element, or a combination thereof.

22. A food, food additive, feed, nutritional supplement, probiotic supplement, personal care product, health care product, and or a veterinary product comprising the defense system of any one of claims 7-10, or the nucleic acid construct of any one of claims 11-16, or the transmissible genetic element of claim 17, or the isolated cell of any one of claims 18-21.

23. The food, food additive, feed, nutritional supplement, probiotic supplement, personal care product, health care product, and or a veterinary product of claim 22, wherein said defense system comprises a combination of Defense Systems selected from Defense Systems Ia-Xc (a)-(o).

24. A method of preparing a food, a food additive, a feed, a nutritional supplements, a probiotic supplement, a personal care product, a health care product or a veterinary product, the method comprising adding to the food, food additive, feed, nutritional supplement, probiotic supplement, personal care product, health care product, or veterinary product at least one defense system of any one of claims 7-10, or the nucleic acid construct of any one of claims 11-16, or the transmissible genetic element of claim 17, or the isolated cell of any one of claims 18-21.

25. A method for identifying a defense system in a prokaryotic cell, comprising the steps of:
(a) selecting, in-silico, a family of genes that are preferentially located in close proximity to a known defense-related gene family present in a plurality of prokaryotic genomes; and
(b) analyzing, in-silico, the DNA upstream and downstream of said gene; and
(c) identifying, in-silico, a cassette of genes within the upstream and downstream DNA analyzed in step (b) comprising said gene of step (a), wherein said cassette of genes has conserved synteny in said plurality of prokaryotic genomes.

26. The method of claim 25, wherein the gene of step (a), located in close proximity to a known defense related gene family, is within 10 genes upstream and or 10 genes downstream of the known defense related gene family.

27. A method gene editing; said method comprising contacting a nucleic acid sequence comprising a gene to be edited with at least one polypeptide component of a defense system, wherein said defense system component comprises a component selected from the defense system of claim 7; where said method results in the gene being edited.
